(19) United States
(12) Patent Application Publication
(10) Pub. No.: US 2024/0228482 A1
(43) Pub. Date: Jul. 11, 2024

(54) COMPOUNDS, COMPOSITIONS AND METHODS OF TREATING CANCER

(71) Applicant: HOTSPOT THERAPEUTICS, INC., Boston, MA (US)

(72) Inventors: Yingzhi Bi, Plainsboro, NJ (US); Ken Carson, Princeton, NJ (US); Geraldine Cirillo Harriman, Charlestown, RI (US); Graham A.B. Hone, Hopewell, NJ (US); Rajiv Gandhi Govindaraj, Mansfield, MA (US); Rajiah Aldrin Denny, Sharon, MA (US); David J. Diller, East Windsor, NJ (US)

(21) Appl. No.: 18/286,966

(22) PCT Filed: Apr. 15, 2022
(86) PCT No.: PCT/US2022/025083

§ 371 Date: Oct. 13, 2023

Related U.S. Application Data
(60) Provisional application No. 63/175,974, filed on Apr. 16, 2021.
(60) Provisional application No. 63/281,493, filed on Nov. 19, 2021.

(51) Int. Cl. C07D 471/04

(52) U.S. Cl. CPC C07D 471/04

(57) ABSTRACT

The present disclose includes, among other things, compounds that treat or lessen the severity of cancer, pharmaceutical compositions and methods of making and using the same.

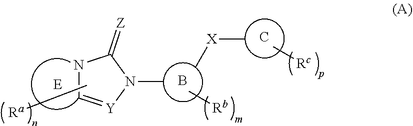

### CROSS REFERENCES TO RELATED APPLICATIONS

This application claims priority to U.S. Provisional Application Nos. 63/175,974 filed Apr. 16, 2021 and 63/281,493 filed Nov. 19, 2021, the contents of each of which are incorporated herein by reference.

### BACKGROUND

Cbl-b is a E3 ubiquitin-protein ligase that functions as a negative regulator of T-cell activation. Modulation of Cbl-b has been shown to be a therapeutic target for a diseases and disorders. There remains a need for compounds that inhibit Cbl-b.

### SUMMARY

In some embodiments, the present disclosure includes a compound of formula (A):

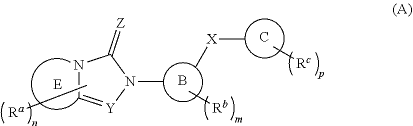

or a pharmaceutically acceptable salt thereof.

Additionally, the present disclosure includes, among other things, pharmaceutical compositions, methods of using and methods of making a compound of formula (A).

### DETAILED DESCRIPTION

In some embodiments, the present disclosure includes a compound of formula (A):

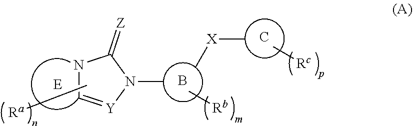

or pharmaceutically acceptable salts thereof,
wherein
Y is selected from the group ═C(H)—, ═C(R^{a})— or ═N—;
Z is ═O or ═S;
E is optionally substituted 5-6 membered heterocyclyl;
B is optionally substituted phenyl, optionally substituted 8-10 membered bicyclyl, or optionally substituted 5-6 membered heteroaryl;
C is optionally substituted 5-6 membered heterocyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from a group consisting of halogen, C₁-C₃ aliphatic, phenyl, 3-6-membered heteroaryl, 3-6-membered heterocylyl, and —(CH₂)(3-6-membered carbocyclyl);
each R^{a} is independently selected from the group consisting of L-Y, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
   or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5;
m is 0, 1, 2, 3, or 4; and
p is 0, 1, 2, 3, or 4.

In some embodiments, the present disclosure includes a compound of Formula (B):

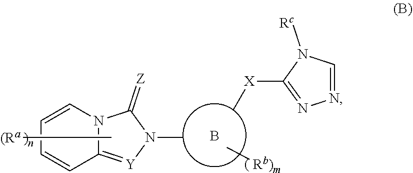

or pharmaceutically acceptable salts thereof,
wherein
Y is selected from the group ═C(H)—, ═C(R^{a})— or ═N—;
Z is ═O or ═S;
B is optionally substituted phenyl, substituted 5-6 membered heteroaryl or optionally substituted 8-10 membered bicyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl,
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, —OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
   or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5; and
m is 0, 1, 2, 3, or 4.

In some embodiments, the present disclosure includes a compound of formula (I):

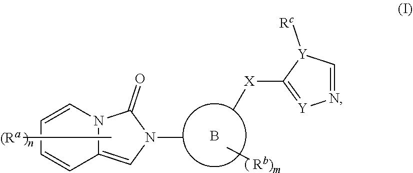

or pharmaceutically acceptable salts thereof,
wherein
X is an optionally substituted C₁-C₃ alkylene chain, wherein one or more methylene units is optionally replaced by —N(H)—, —N(R¹)—, —O—, —S—, —SO—, —SO₂—,

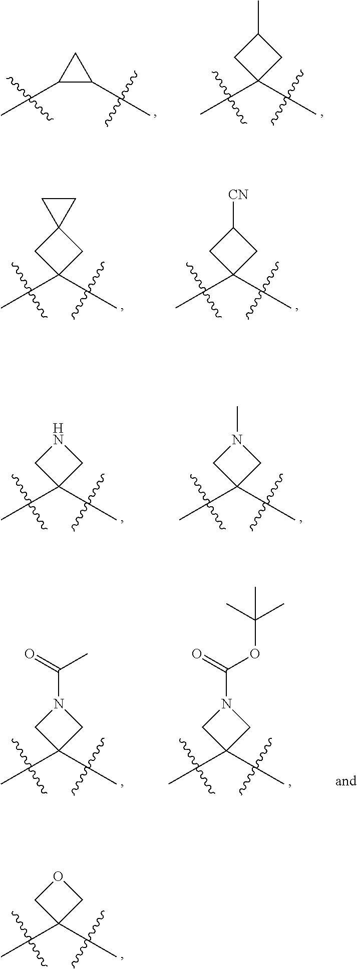

and wherein each methylene unit may be substituted with 1-2 substituents independently selected from the group consisting of halogen, optionally substituted C₁-C₃ aliphatic, optionally substituted 5-membered heteroaryl, optionally substituted phenyl, optionally substituted C₃-C₄ carbocylyl, and optionally substituted C₃-C₄ heterocyclyl;
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
each Y is independently selected from the group consisting of —C═, —O—, —N═, and —S—;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
B is optionally substituted phenyl, substituted 5-6 membered heteroaryl, or optionally substituted 8-10 membered bicyclyl;
each R^{b} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, wherein the—optionally deuterated optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
   or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5; and
m is 0, 1, 2, 3, or 4.

In some embodiments, present disclosure includes a compound is of formula (Ia) or (IIa):

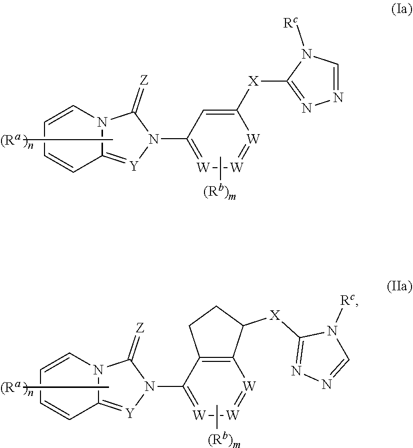

or pharmaceutically acceptable salts thereof,
wherein each W is independently selected from N or C; and
X, Y, Z, R^{a}, R^{b}, R^{c}, n, and m are defined above and described in classes and subclasses herein.

In some embodiments, present disclosure includes a compound is of formula (Ia1) or (IIa1):

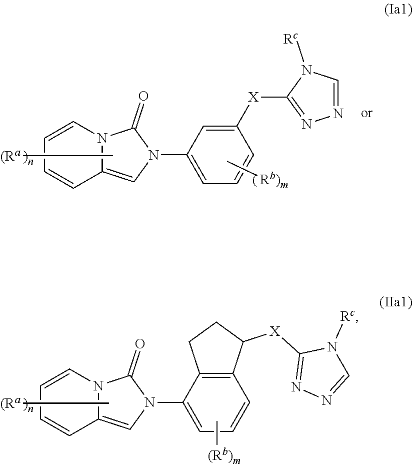

or a pharmaceutically acceptable salt thereof, wherein X, Y, Z, R^{a}, R^{b}, R^{c}, and n are defined above and described in classes and subclasses herein.

In some embodiments, present disclosure includes a compound is of formula (Ia2), (Ia3), or (Ia4):

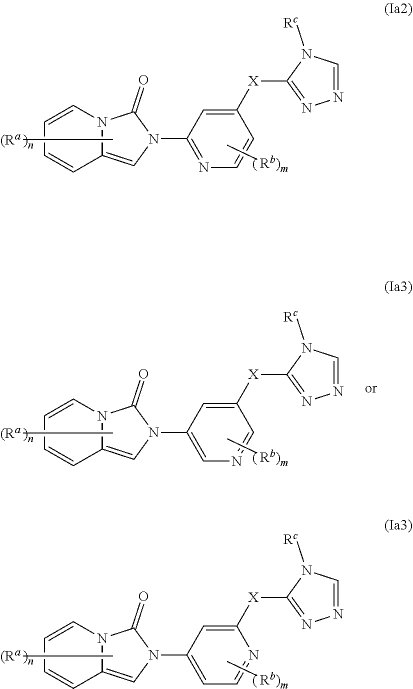

or pharmaceutically acceptable salts thereof, wherein X, Y, Z, R^{a}, R^{b}, R^{c}, n, and n are defined above and described in classes and subclasses herein.

In some embodiments, present disclosure includes a compound is of formula (Ia1) or (IIa1):

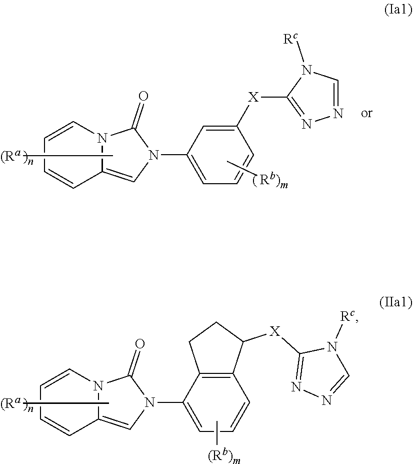

or pharmaceutically acceptable salts thereof,
wherein
X is an optionally substituted C₁-C₃ alkylene chain, wherein one or more methylene units is optionally replaced by —N(H)—, —N(R¹)—, —O—, —S—, —SO—, —SO₂—,

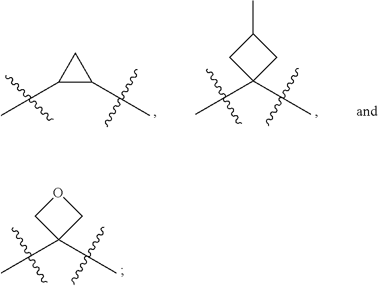

each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
   or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5; and
m is 0, 1, 2, 3, or 4.

In some embodiments, present disclosure includes a compound is of formula (Ib) or (IIb):

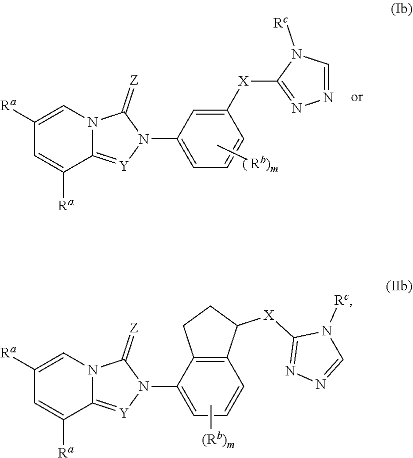

or pharmaceutically acceptable salts thereof, wherein X, Y, Z, R^{a}, R^{b}, R^{c}, and m are defined above and described in classes and subclasses herein.

In some embodiments, present disclosure includes a compound of formula (Ib1) or (IIb1):

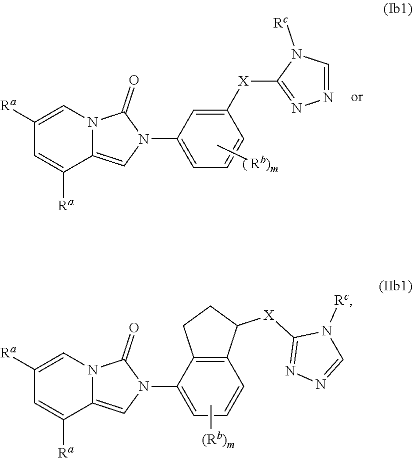

or a pharmaceutically acceptable salt thereof, wherein X, R^{a}, R^{b}, R^{c} and m are defined above and described in classes and subclasses herein.

In some embodiments, present disclosure includes a compound of formula (Ib2), (Ib3), or (Ib4):

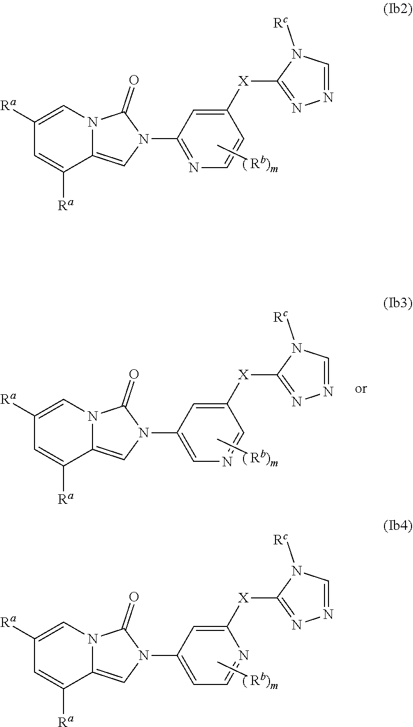

or a pharmaceutically acceptable salt thereof, wherein X, R^{a}, R^{b}, R^{c} and m are defined above and described in classes and subclasses herein.

In some embodiments, present disclosure includes a compound of formula (Ic) or (IIc):

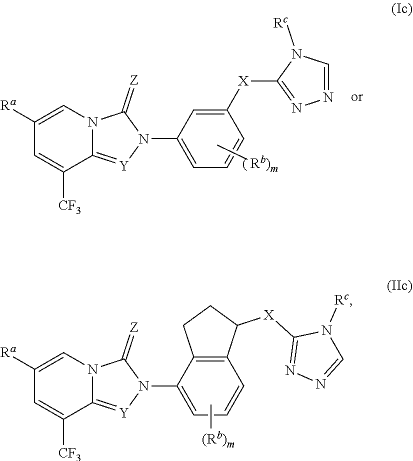

or a pharmaceutically acceptable salt thereof, wherein X, Y, Z, R^{a}, R^{b}, R^{c} and m are defined above and described in classes and subclasses herein.

In some embodiments, present disclosure includes a compound of formula (Ic1) or (IIc1):

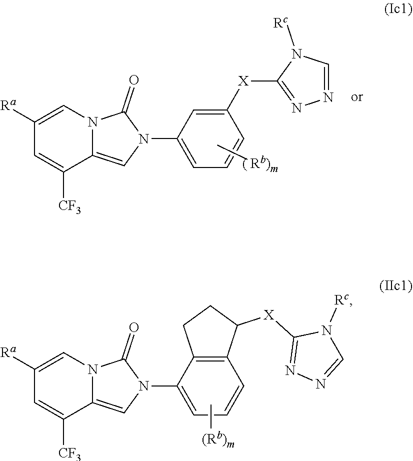

or a pharmaceutically acceptable salt thereof, wherein X, R^{a}, R^{b}, R^{c} and m are defined above and described in classes and subclasses herein.

In some embodiments, present disclosure includes a compound of formula (Id) or (IId):

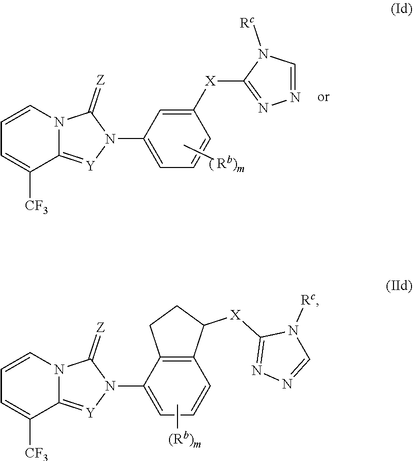

or a pharmaceutically acceptable salt thereof, wherein X, R^{b}, R^{c} and m are defined above and described in classes and subclasses herein.

In some embodiments, present disclosure includes a compound of formula (Id1) or (IId1):

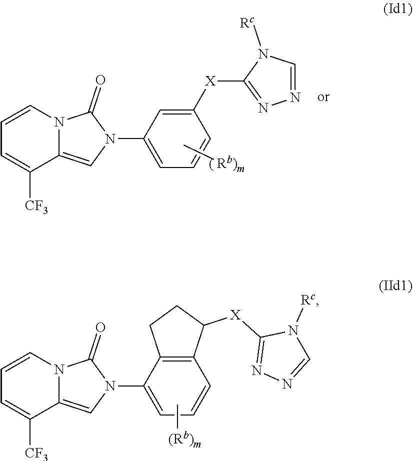

or a pharmaceutically acceptable salt thereof, wherein X, R^{b}, R^{c} and m are defined above and described in classes and subclasses herein.

### X

In some embodiments, X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from the group consisting of halogen, C₁-C₃ aliphatic, phenyl, 3-6-membered heteroaryl, 3-6-membered heterocylyl, and —(CH₂)(3-6-membered carbocyclyl). In some embodiments, X is an optionally substituted C₁-C₃ alkylene chain, wherein one or more methylene units is optionally replaced by —N(H)—, —N(R¹)¹, —O—, —S—, —SO—, —SO₂—, optionally substituted 3-6-membered carbocyclyl, and optionally substituted 3-6-membered heterocylyl. In some embodiments, X is an optionally substituted C₁-C₃ alkylene chain, wherein one or more methylene units is optionally replaced by —N(H)—, —N(R¹), —O—, —S—, —SO—, —SO₂—,

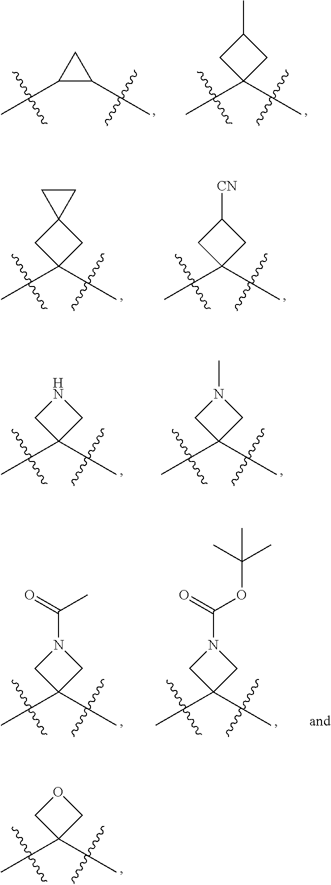

and wherein each methylene unit may be substituted with 1-2 substituents independently selected from the group consisting of halogen, optionally substituted C₁-C₃ aliphatic, optionally substituted 5-membered heteroaryl, optionally substituted phenyl, optionally substituted C₃-C₄ carbocylyl, and optionally substituted C₃-C₄ heterocyclyl. In some embodiments, In some embodiments, X is an optionally substituted C₁-C₃ alkylene chain, wherein one or more methylene units is optionally replaced by —N(H)—, —N(R¹)—, —O—, —S—,

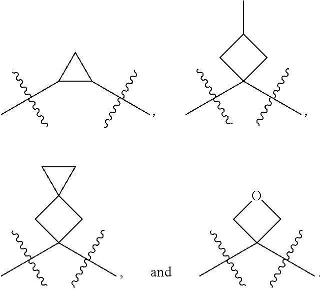

In some embodiments, X is an optionally substituted C₁-C₃ alkylene chain, wherein one or more methylene units is optionally replaced by —N(H)—, —N(R¹)—, —O—, —S—, —SO—, —SO₂—,

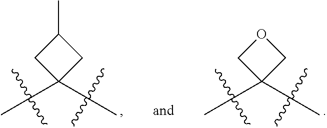

In some embodiments, X is optionally substituted C₁-C₂ alkylene. In some embodiments X is

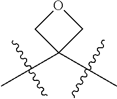 [SMILES: *C1(*)COC1]

or optionally substituted C₂ alkylene, wherein one methylene unit is replaced with

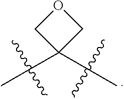 [SMILES: *C1(*)COC1]

In some embodiments, X is selected from the group consisting of

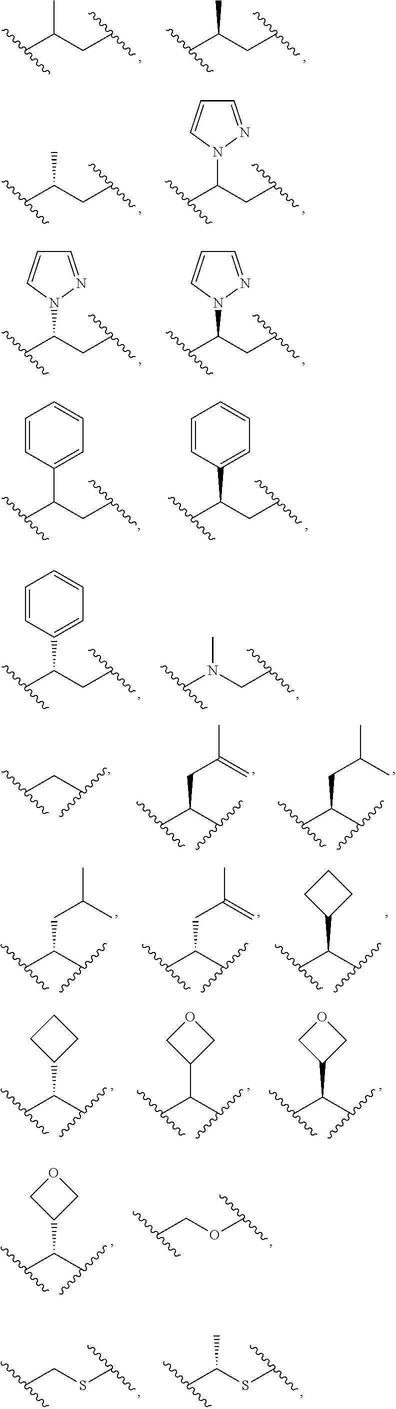

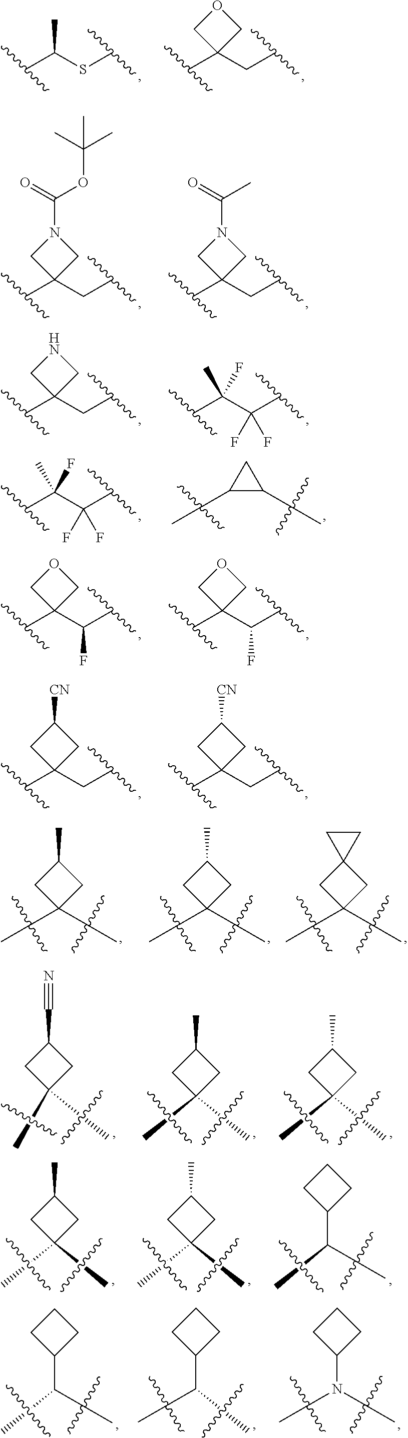

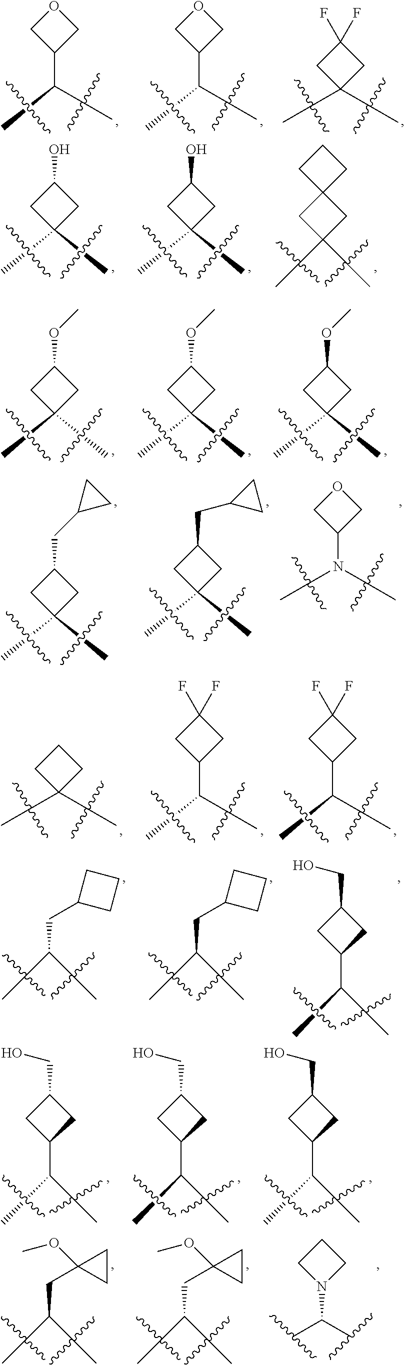

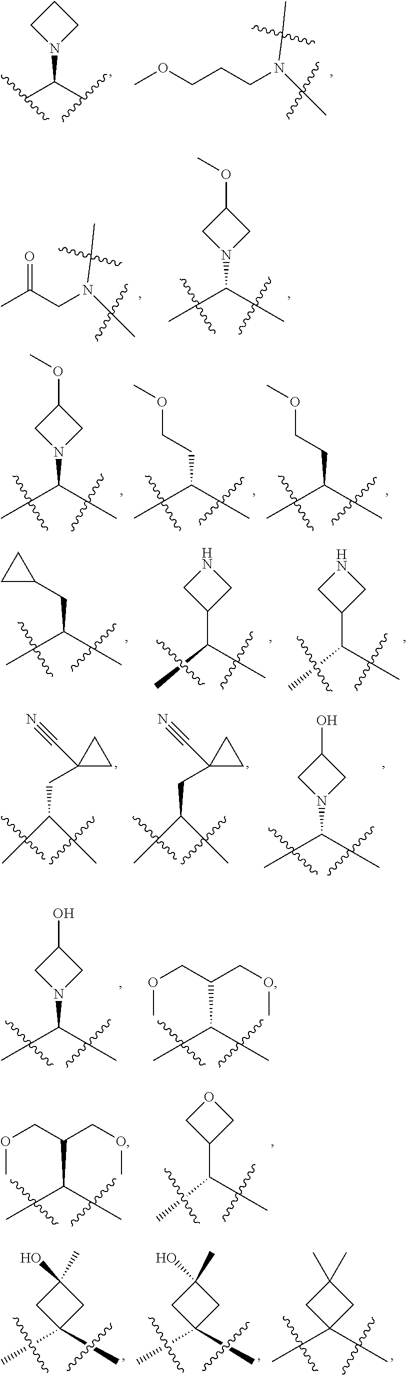

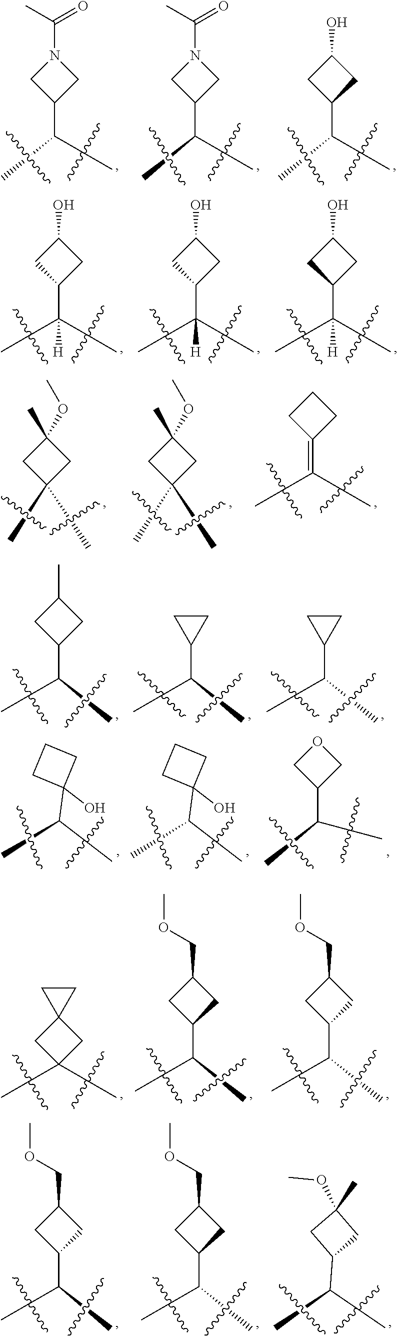

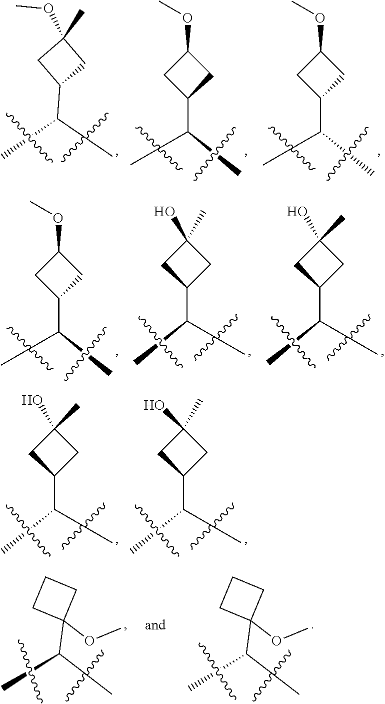

In some embodiments, wherein X is selected from the group consisting of

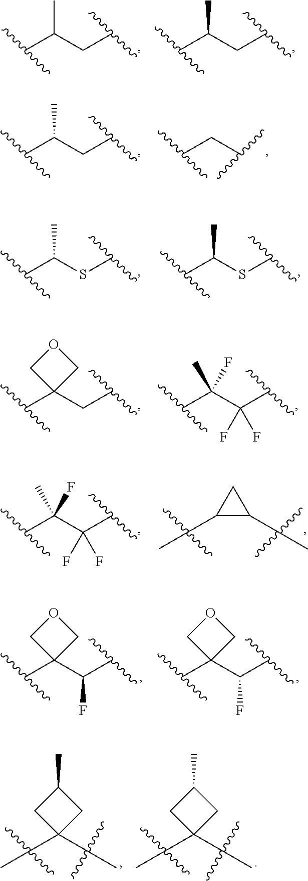

### R^{a}

In some embodiments, each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S. In some embodiments, L-A. In some embodiments, R^{a} is selected from halogen, —CN, —C(O)R¹, —CO₂H, —CONR¹R², optionally substituted C₁-C₆ aliphatic, and optionally substituted C₁-C₆ heteroalkyl. In some embodiments each R^{a} is independently selected from the group consisting of halogen, —CN, —CO₂H, —CHO, —CHF₂, —CF₃, —OMe, —S(O)₂NHMe,

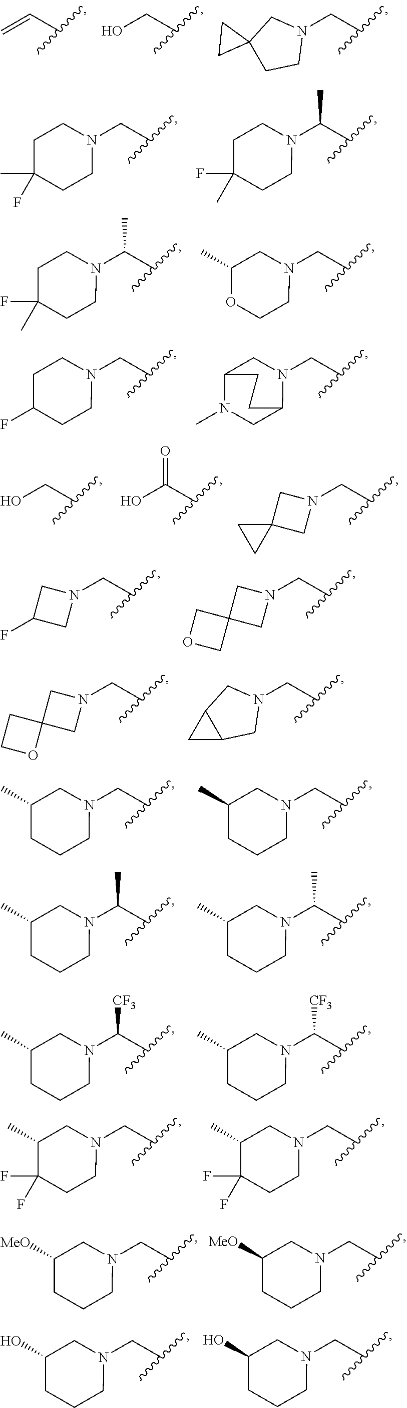

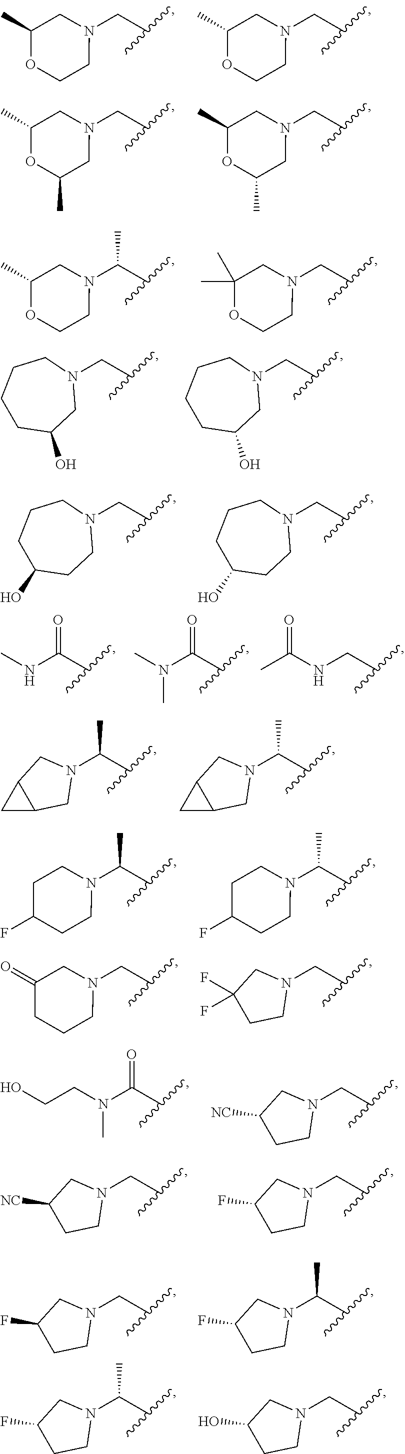

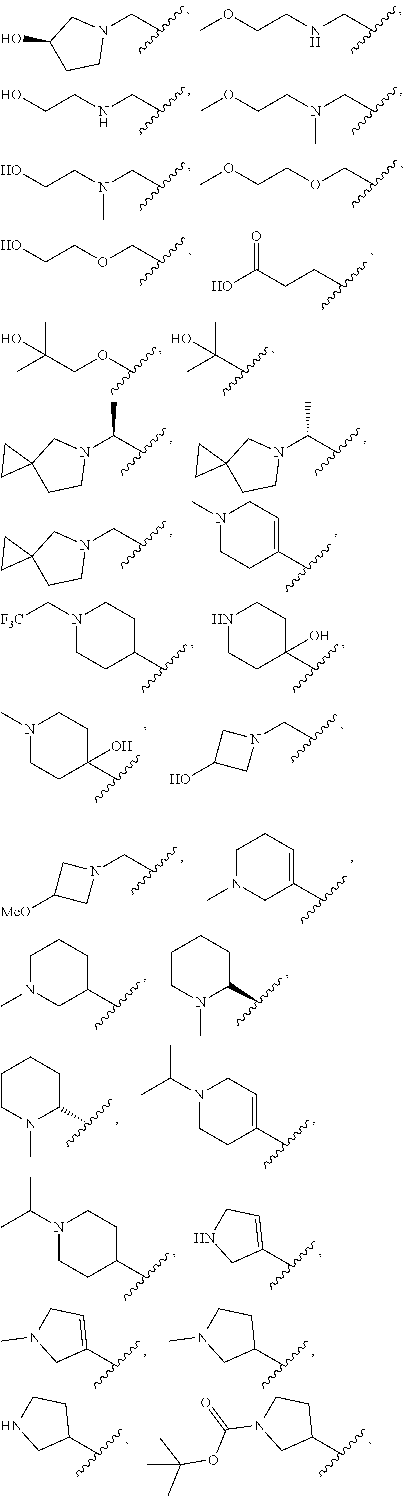

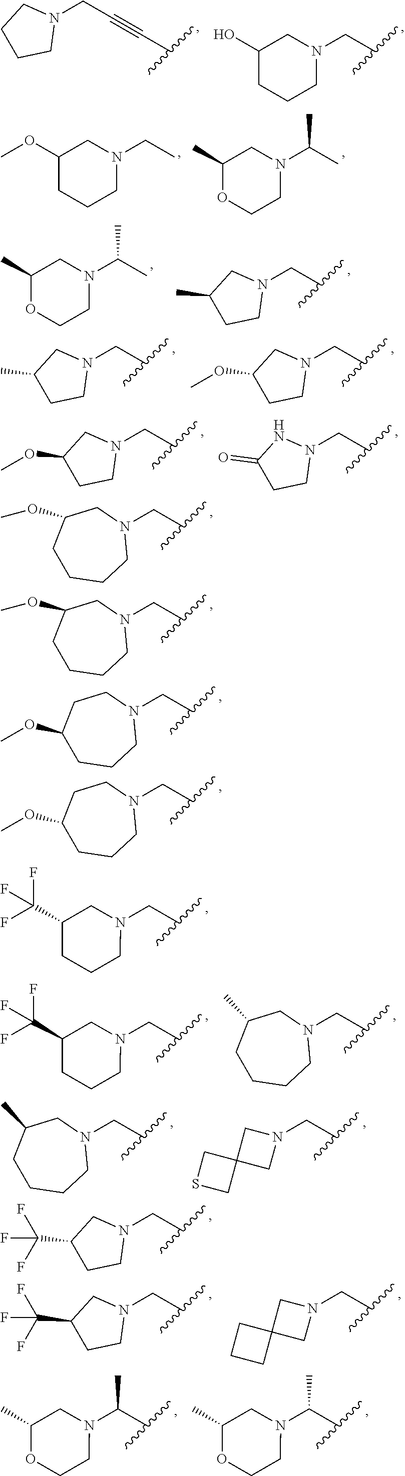

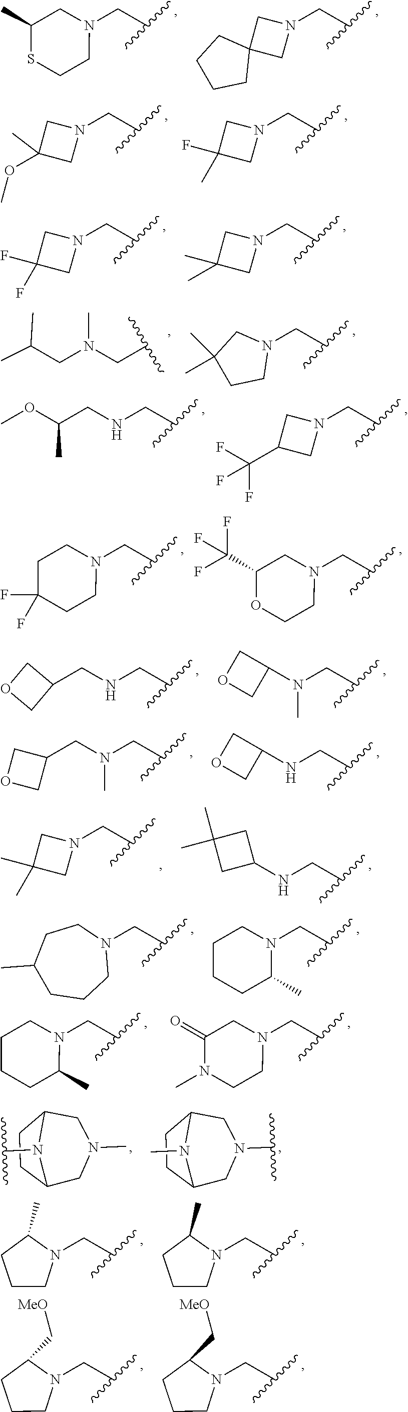

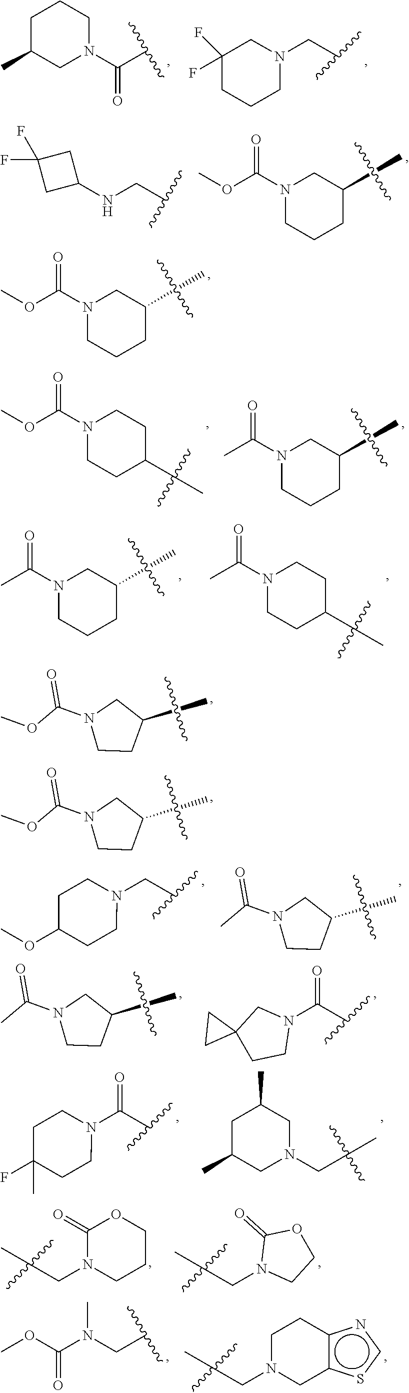

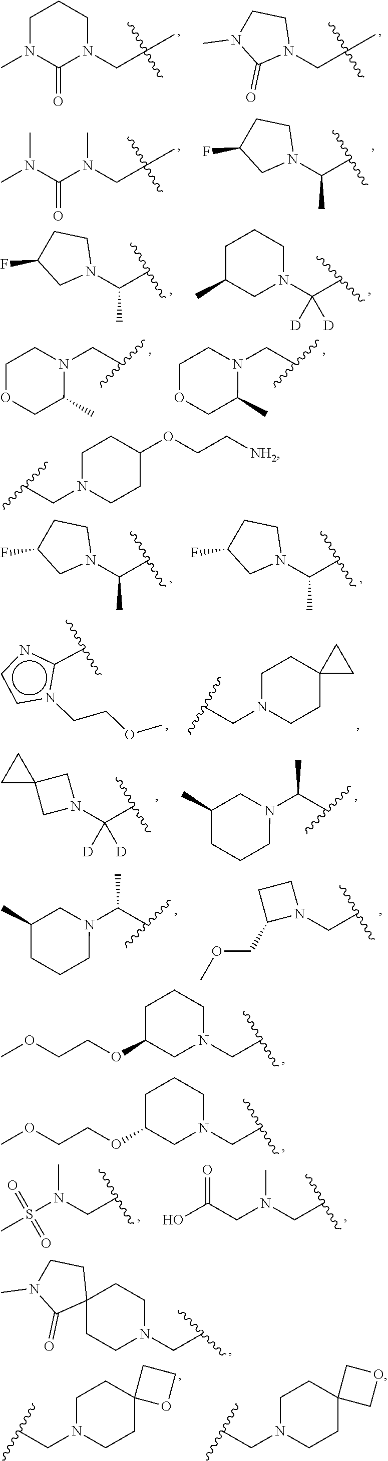

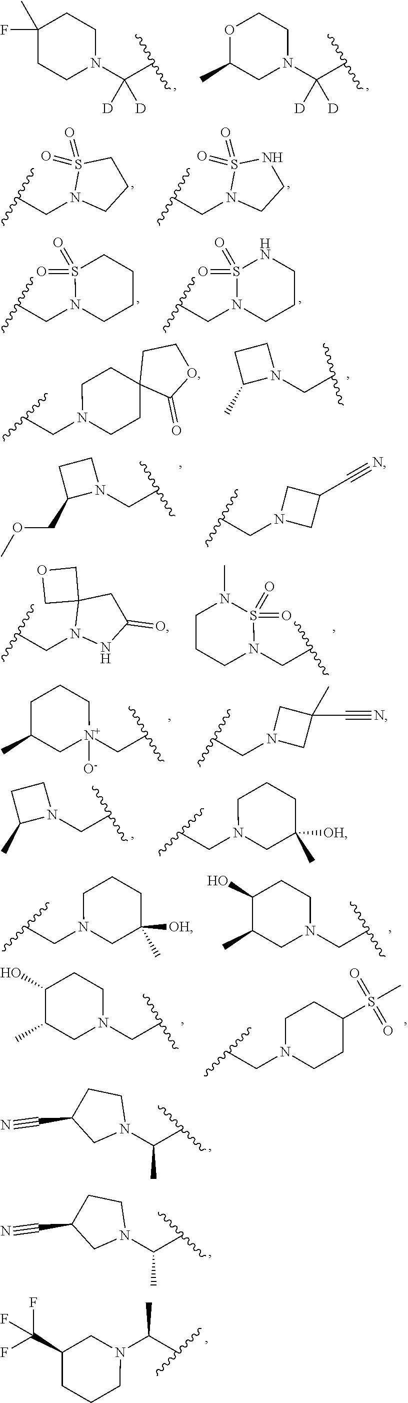

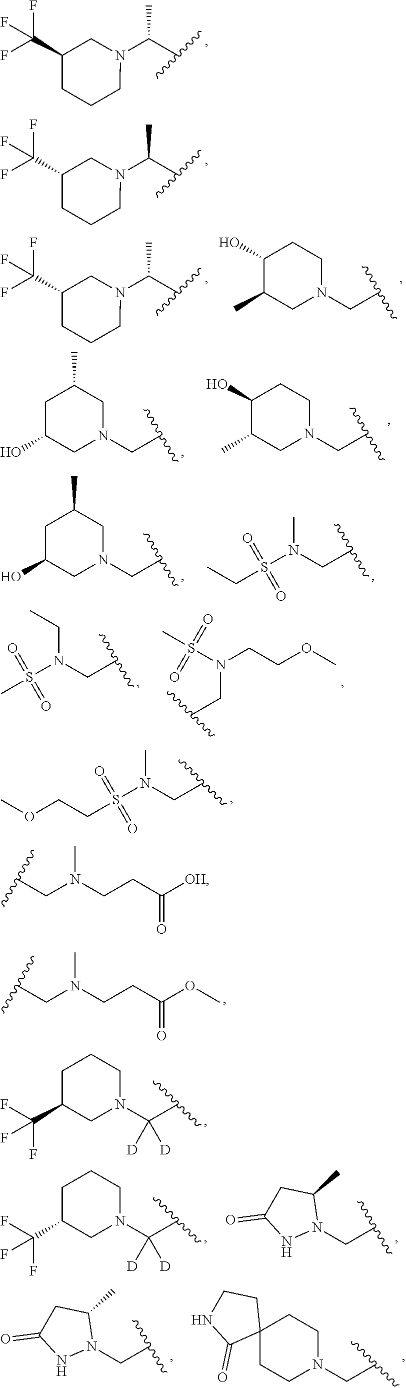

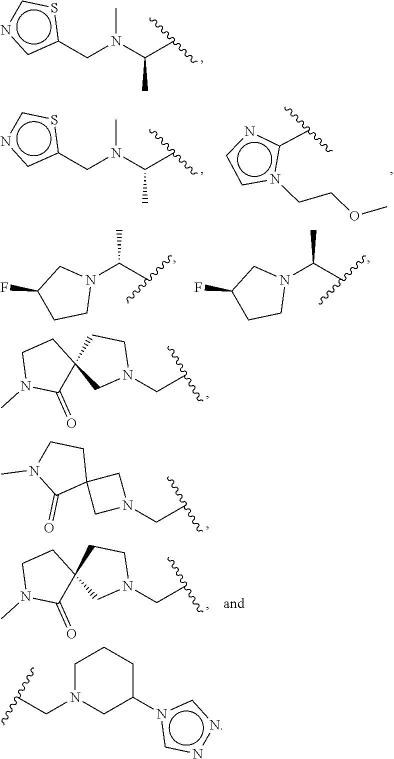

In some embodiments, R^{a} is selected from the group consisting of halogen, —CN, —CO₂H,

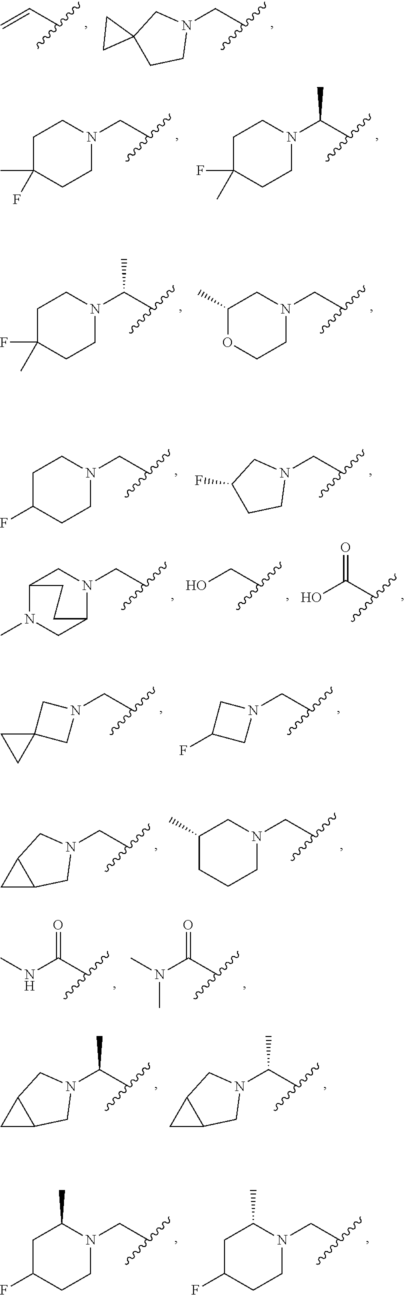

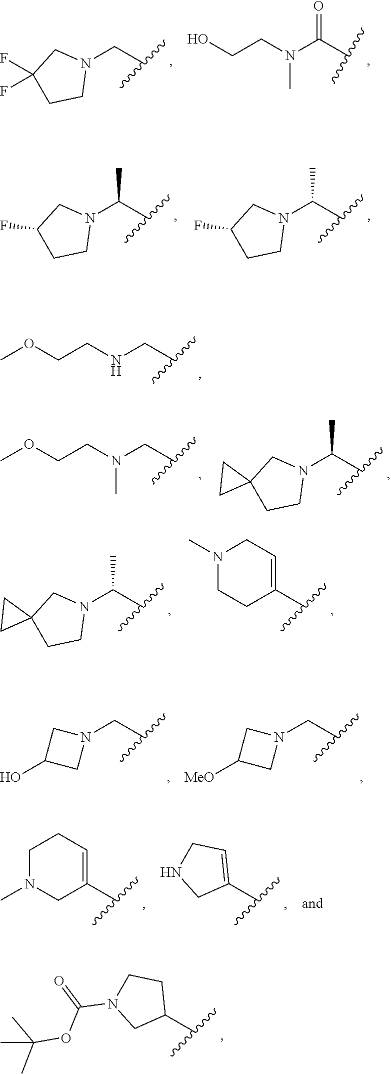

### L

In some embodiments, L is an optionally substituted C₁-C₃ alkylene chain. In some embodiments, L is —CH₂— or —CH(CH₃)—.

### A

In some embodiments, A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S. In some embodiments, A is optionally substituted 3-6 membered heterocyclyl containing 1-4 heteroatoms each selected from the group consisting of N, O, and S. In some embodiments, A is selected from optionally substituted piperidine, optionally substituted tetrahydropyridine, optionally substituted pyrrolidine, optionally substituted dihydropyrrole, optionally substituted aziridine, and optionally substituted morpholine.

### C

In some embodiments, C is optionally substituted 5-membered heteroaryl. In some embodiments, C is optionally substituted 5-membered heteroaryl containing 3 nitrogen atoms. In some embodiments, C is optionally substituted triazolyl. In some embodiments, C is optionally substituted 1,2,4 trizaolyl. In some embodiments, C is optionally substituted 1,2,3 trizaolyl. In some embodiments, C is optionally substituted 5-membered heteroaryl containing 2 nitrogen atoms. In some embodiments, C is optionally substituted pyrazolyl. In some embodiments, C is optionally substituted isoxazolyl. In some embodiments, C is optionally substituted thiazolyl. In some embodiments, C is optionally substituted thiadizolyl. In some embodiments, C is optionally substituted 1,3,4 thiadizolyl. In some embodiments, C is optionally substituted pyridinyl. In some embodiments, C is optionally substituted pyrazinyl. In some embodiments, C is optionally substituted pyrimidinyl. In some embodiments, C is optionally substituted pyridazinyl.

### R^{b}

In some embodiments, each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S.

### R^{c}

In some embodiments, each R^{c} is independently selected from the group consisting of hydrogen, optionally substituted C₁-C₆ aliphatic, —OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R^{c} is independently selected from the group consisting of hydrogen, optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, R^{c} is optionally substituted C₁-C₃ aliphatic. In some embodiments, R^{c} is methyl.

### R¹

In some embodiments, each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R¹ is optionally substituted C₁-C₆ aliphatic. In some embodiments, each R¹ is methyl.

### R²

In some embodiments, each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.

In some embodiments, each R² is optionally substituted C₁-C₆ aliphatic. In some embodiments, each R² is methyl.

### R³

In some embodiments, each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes compounds described in Table 1.

TABLE 1

| Cmpd | |
|---|---|
| No. | Structure |
| 1 | 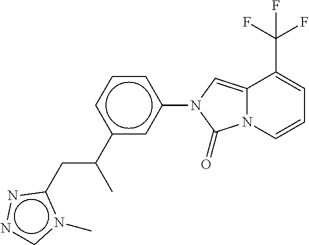 |
| 2 | 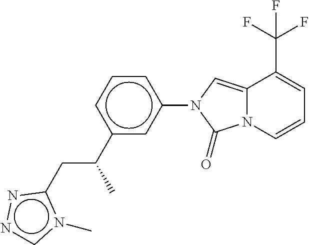 |
| 3 | 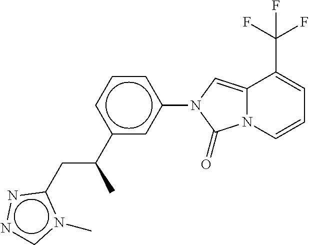 |
| 4 | 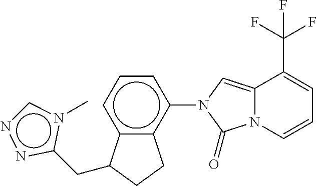 |
| 5 | 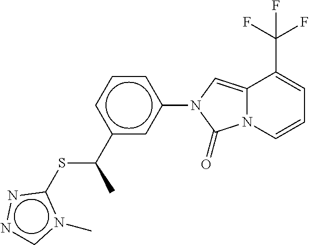 |
| 6 | 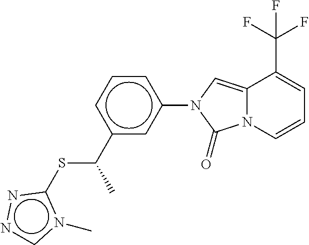 |
| 7 | 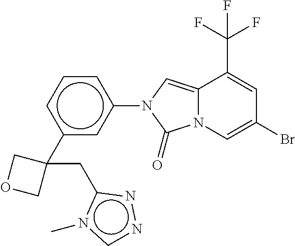 |
| 8 | 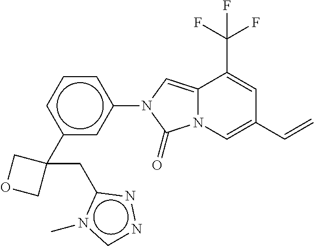 |
| 9 | 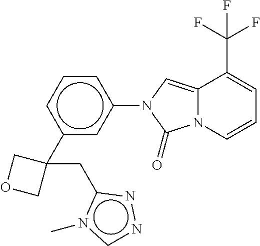 |
| 10 | 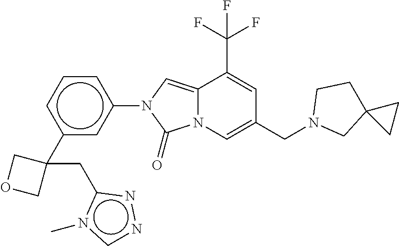 |
| 11 | 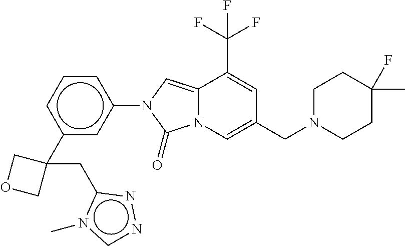 |
| 12 | 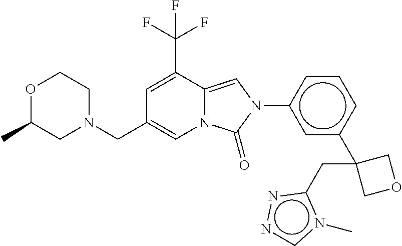 |
| 13 | 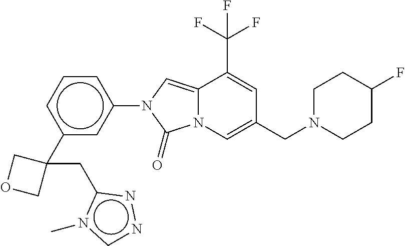 |
| 14 | 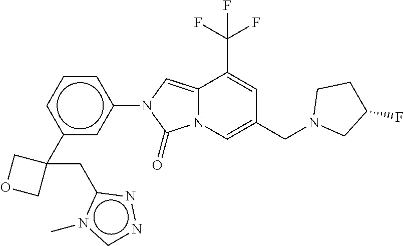 |
| 15 | 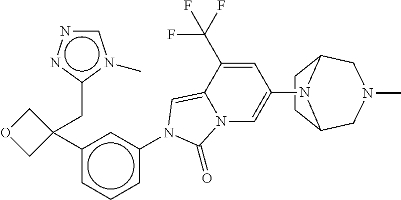 |
| 16 | 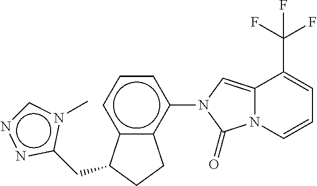 |
| 17 | 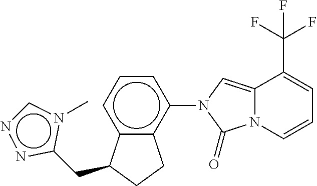 |
| 18 | 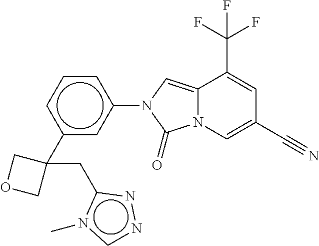 |
| 19 | 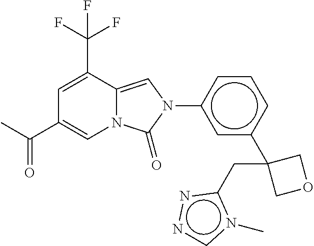 |
| 20 | 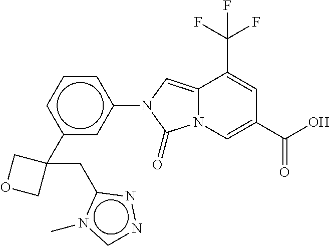 |
| 21 | 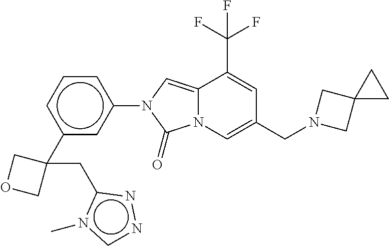 |
| 22 | 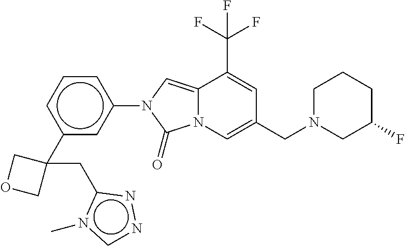 |
| 23 | 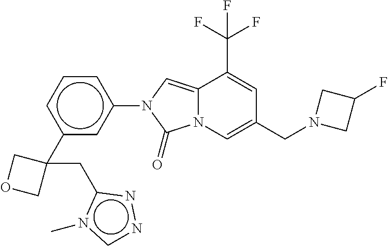 |
| 24 | 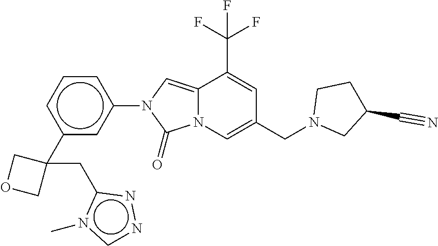 |
| 25 | 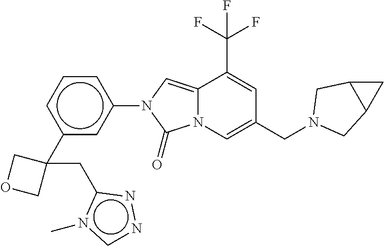 |
| 26 | 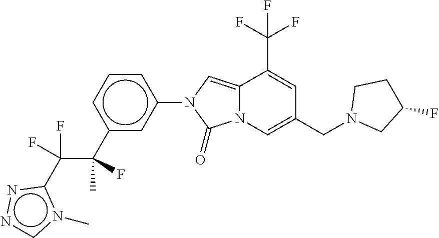 |
| 27 | 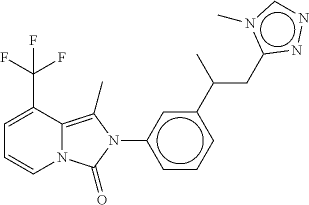 |
| 28 | 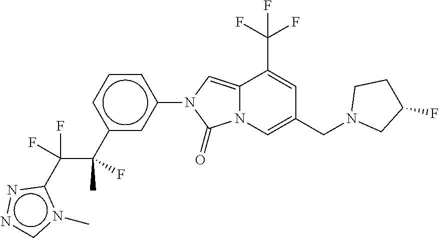 |
| 29 | 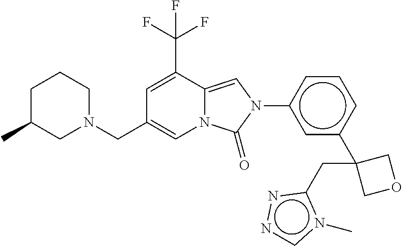 |
| 30 | 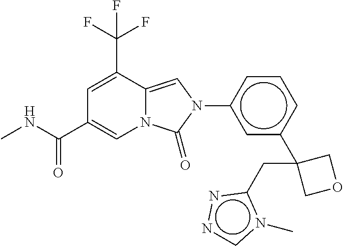 |
| 31 | 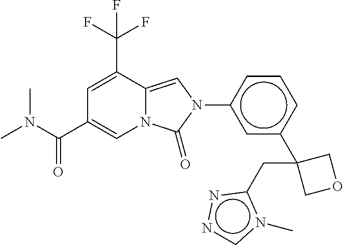 |
| 32 | 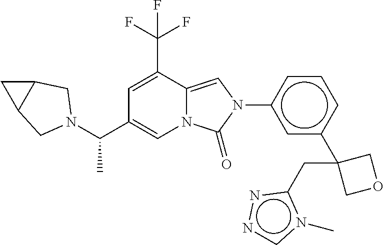 |
| 33 | 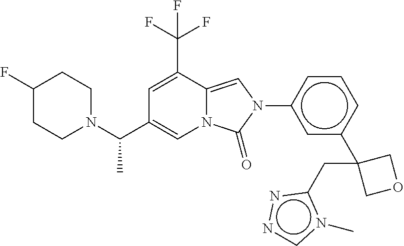 |
| 34 | 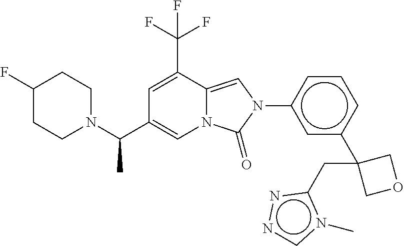 |
| 35 | 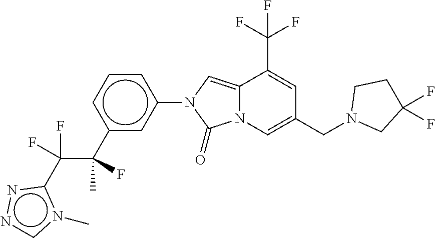 |
| 36 | 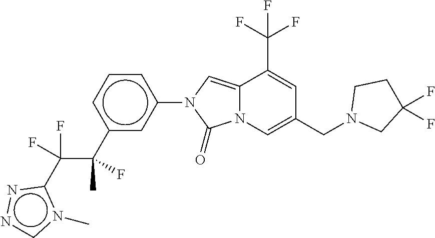 |
| 37 | 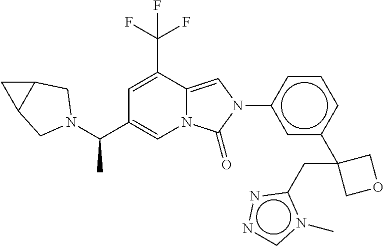 |
| 38 | 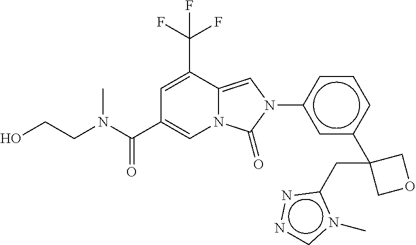 |
| 39 | 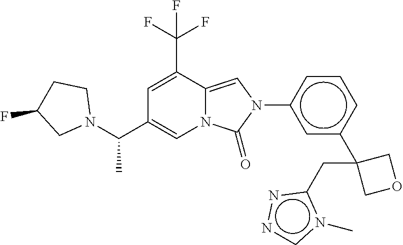 |
| 40 | 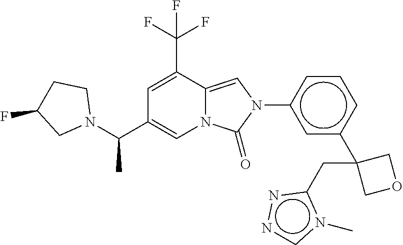 |
| 41 | 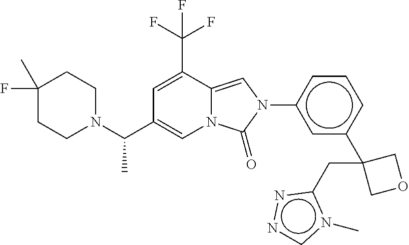 |
| 42 | 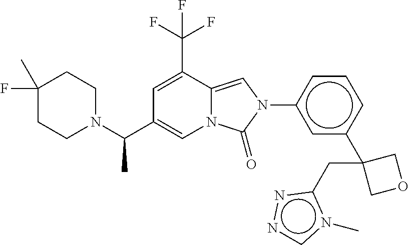 |
| 43 | 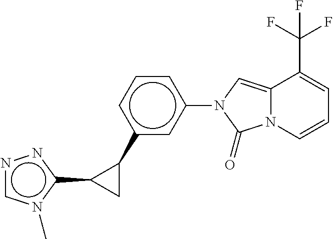 |
| 44 | 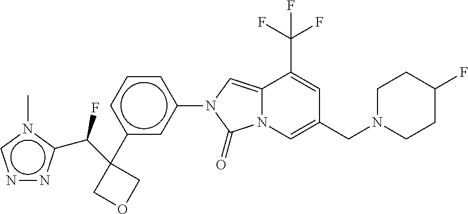 |
| 45 | 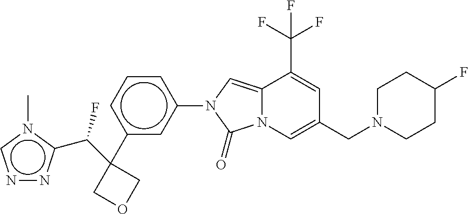 |
| 46 | 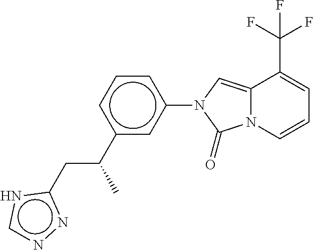 |
| 47 | 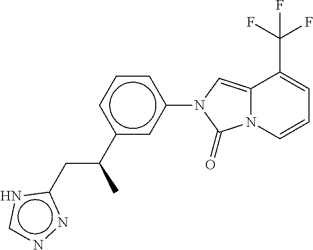 |
| 48 | 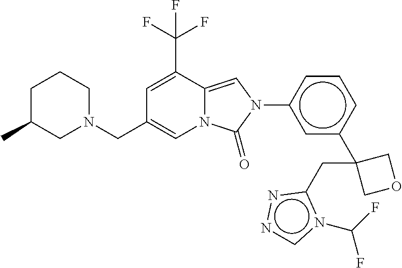 |
| 49 | 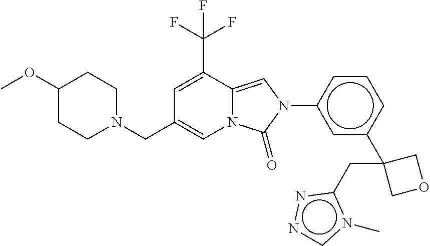 |
| 50 | 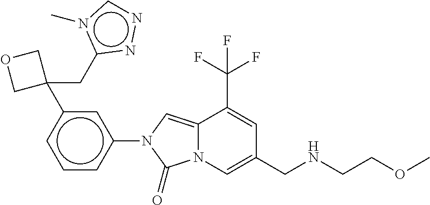 |
| 51 | 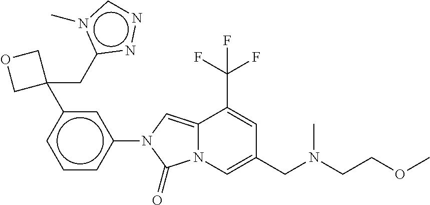 |
| 52 | 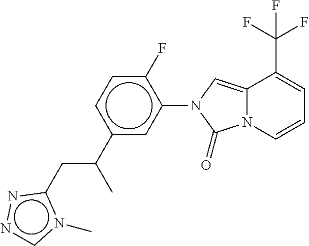 |
| 53 | 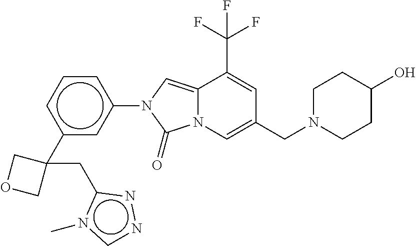 |
| 54 | 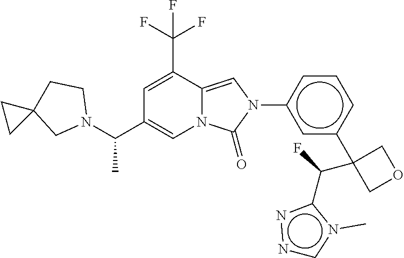 |
| 55 | 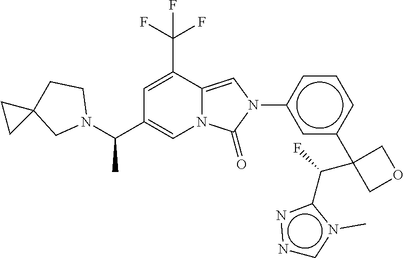 |
| 56 | 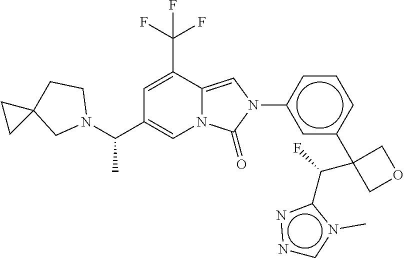 |
| 57 | 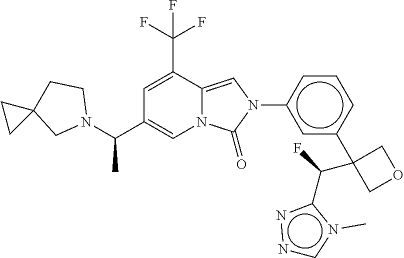 |
| 58 | 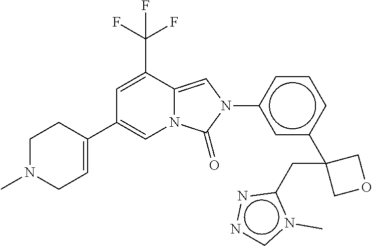 |
| 59 | 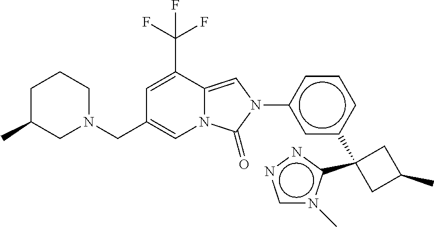 |
| 60 | 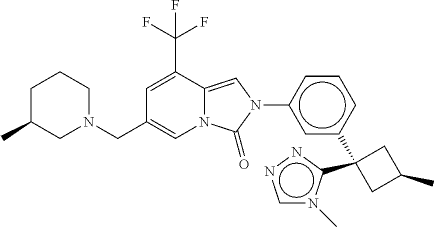 |
| 61 | 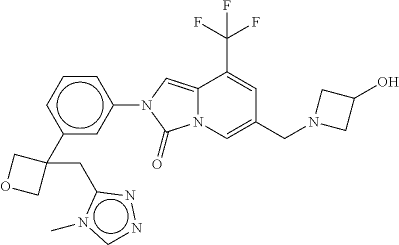 |
| 62 | 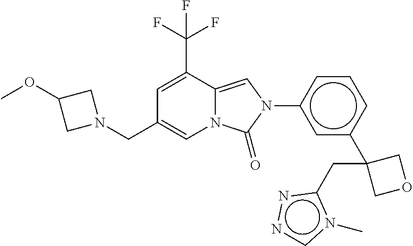 |
| 63 | 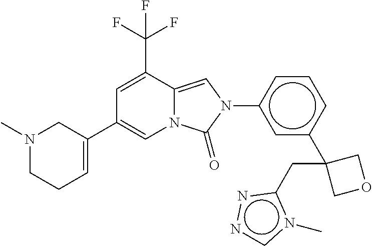 |
| 64 | 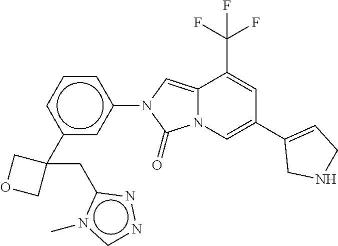 |
| 65 | 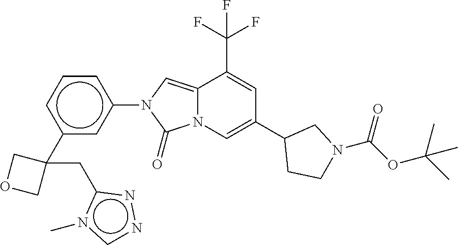 |
| 66 | 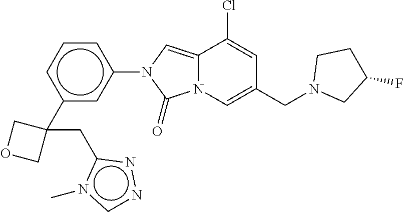 |
| 67 | 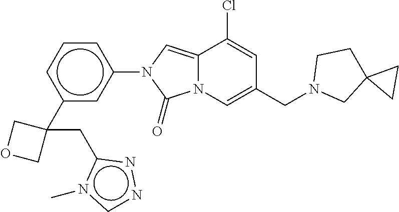 |
| 68 | 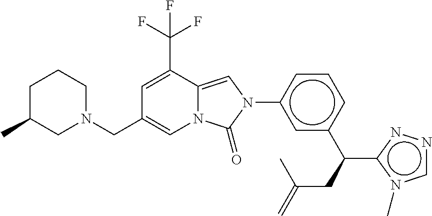 |
| 69 | 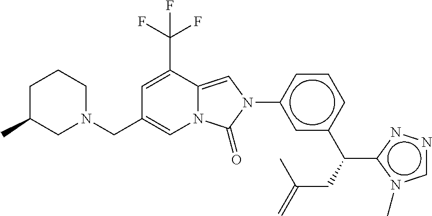 |
| 70 | 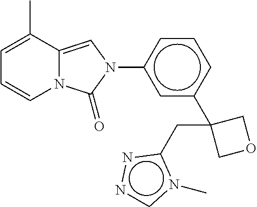 |
| 71 | 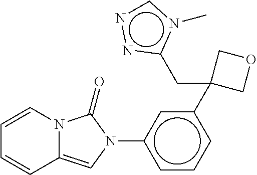 |
| 72 | 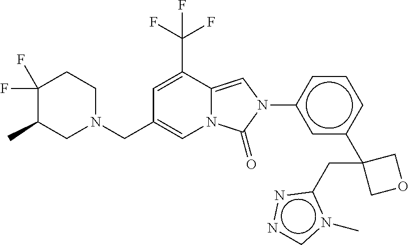 |
| 73 | 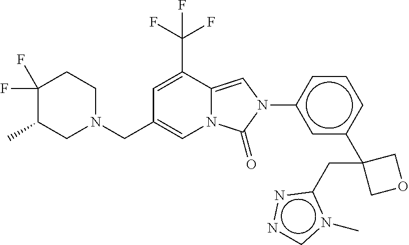 |
| 74 | 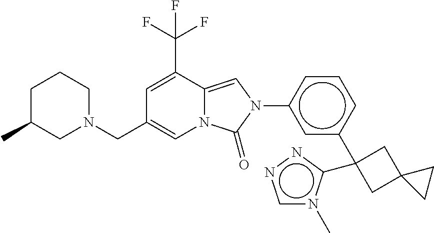 |
| 75 | 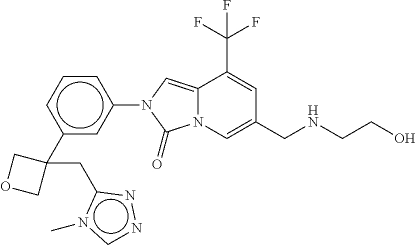 |
| 76 | 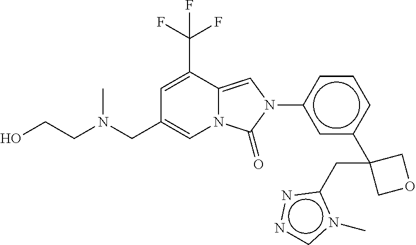 |
| 77 | 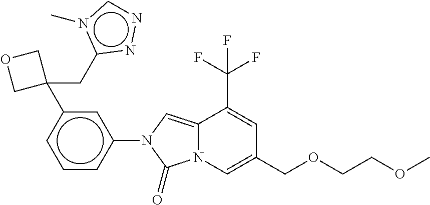 |
| 78 | 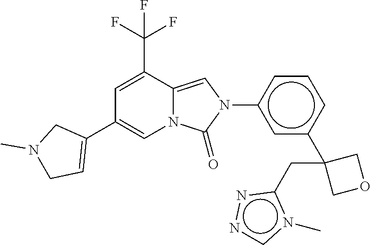 |
| 79 | 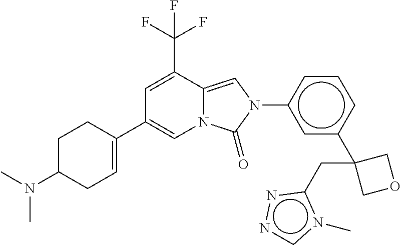 |
| 80 | 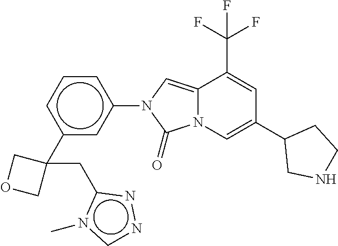 |
| 81 | 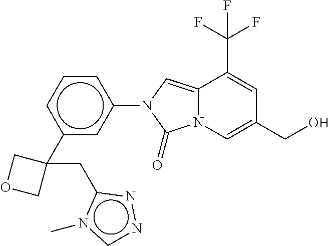 |
| 82 | 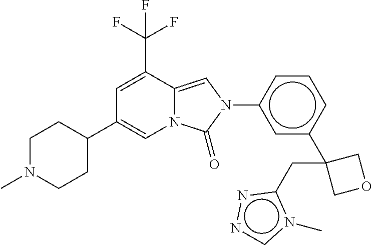 |
| 83 | 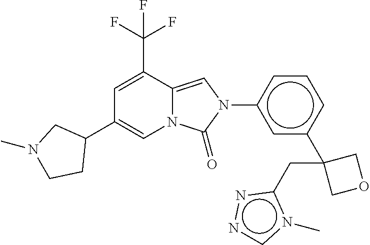 |
| 84 | 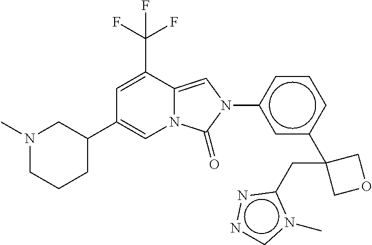 |
| 85 | 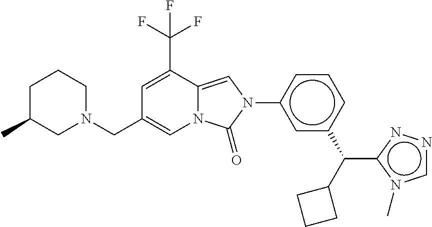 |
| 86 | 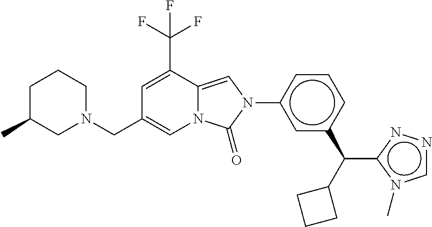 |
| 87 | 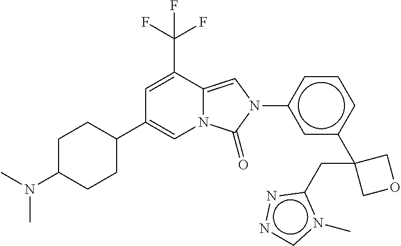 |
| 88 | 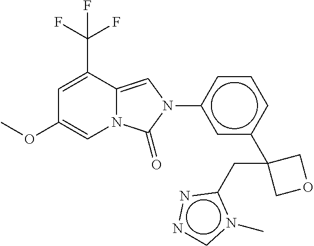 |
| 89 | 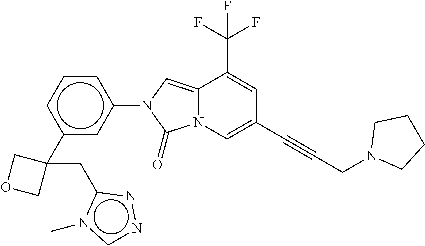 |
| 90 | 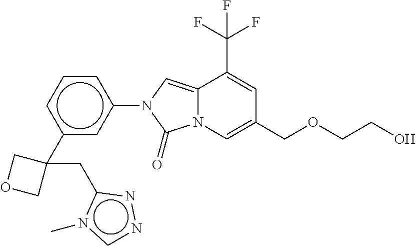 |
| 91 | 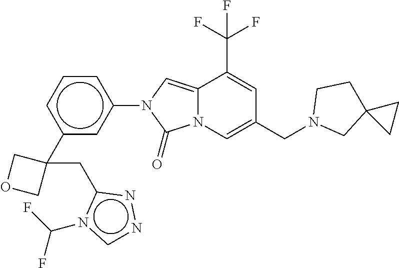 |
| 92 | 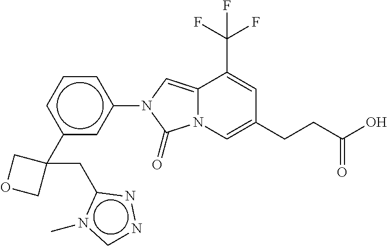 |
| 93 | 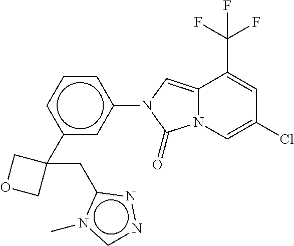 |
| 94 | 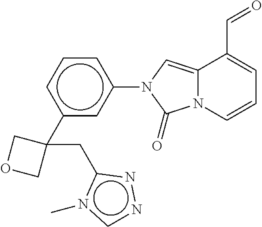 |
| 95 | 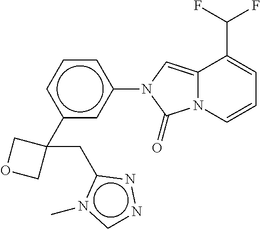 |
| 96 | 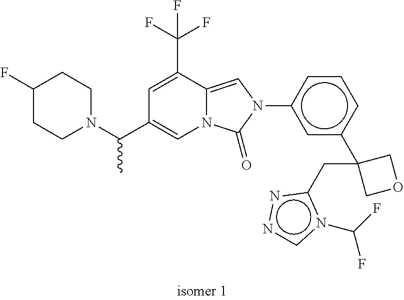 |
| 97 | 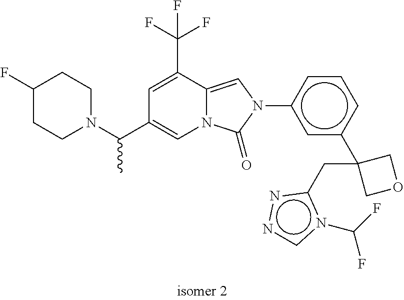 |
| 98 | 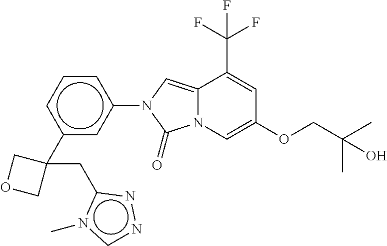 |
| 99 | 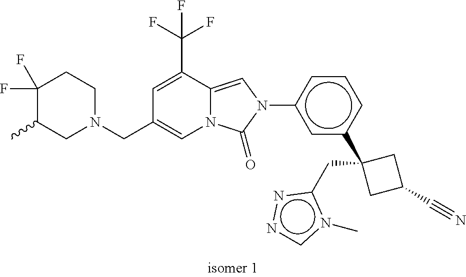 |
| 100 | 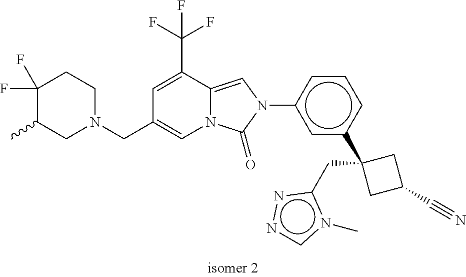 |
| 101 | 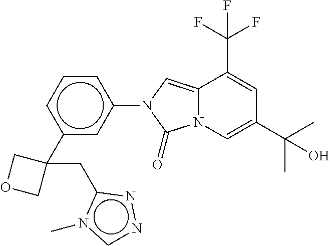 |
| 102 | 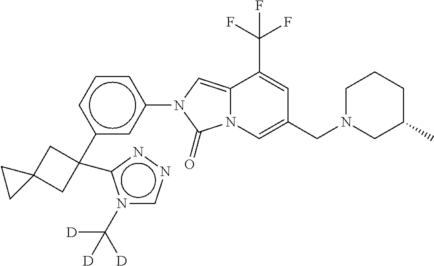 |
| 103 | 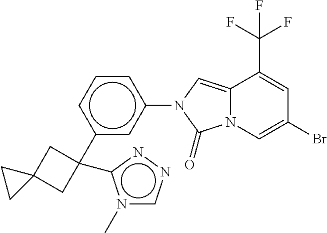 |
| 104 | 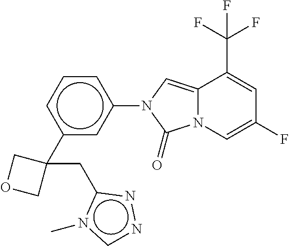 |
| 105 | 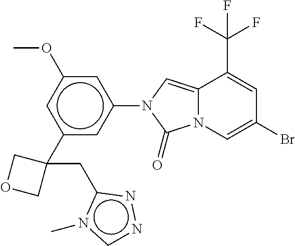 |
| 106 | 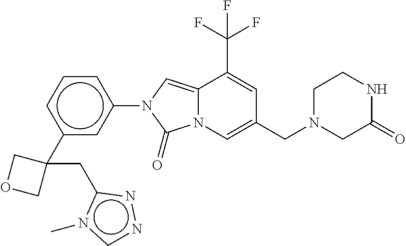 |
| 107 | 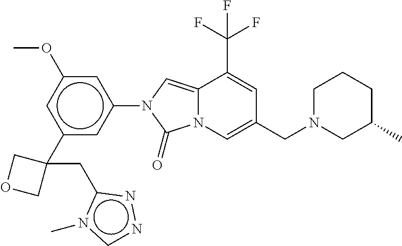 |
| 108 | 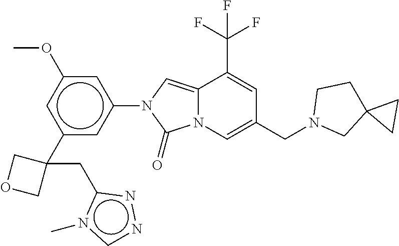 |
| 109 | 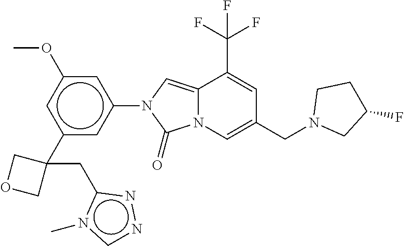 |
| 110 | 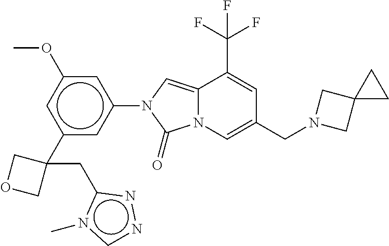 |
| 111 | 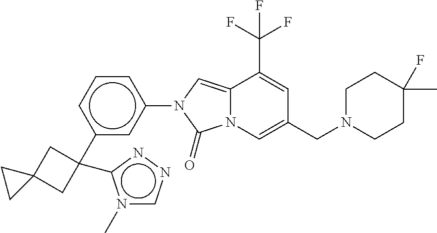 |
| 112 | 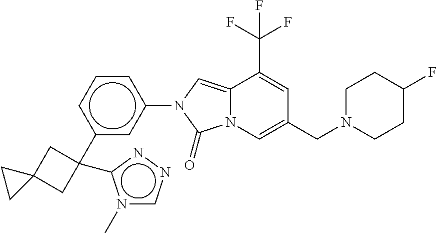 |
| 113 | 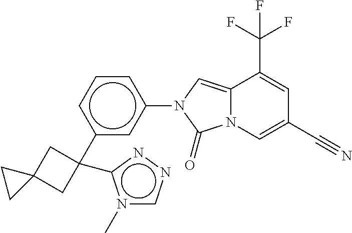 |
| 114 | 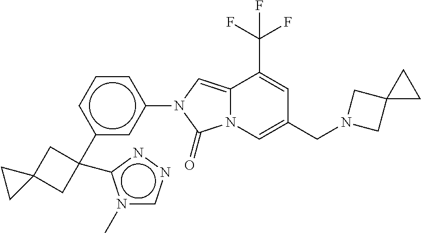 |
| 115 | 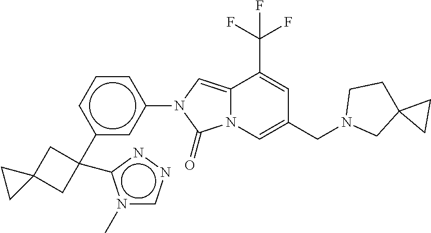 |
| 116 | 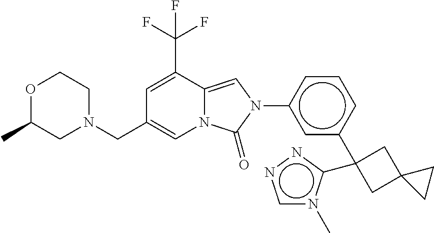 |
| 117 | 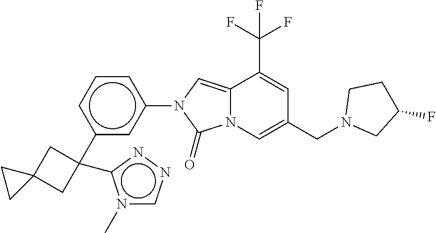 |
| 118 | 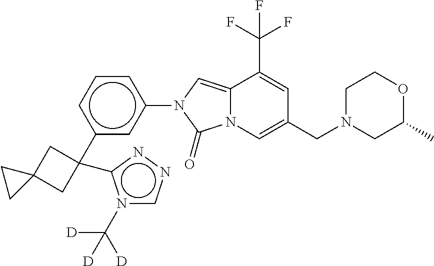 |
| 119 | 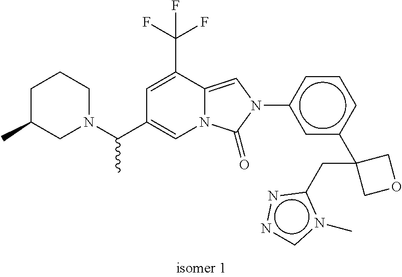 |
| 120 | 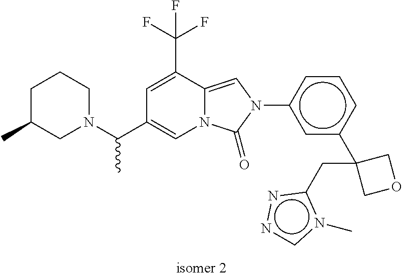 |
| 121 | 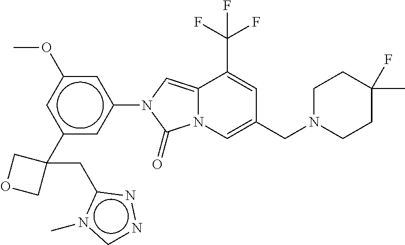 |
| 122 | 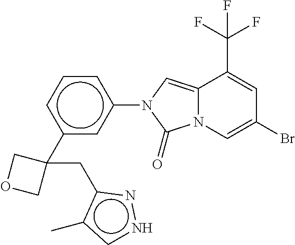 |
| 123 | 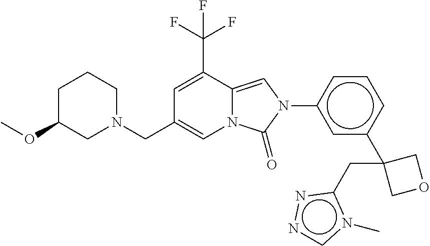 |
| 124 | 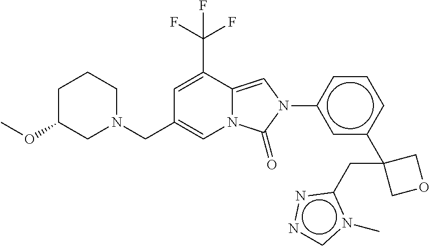 |
| 125 | 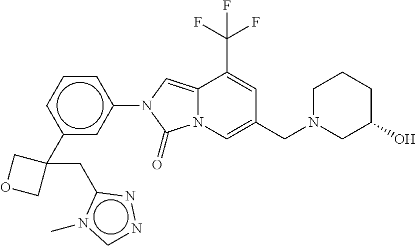 |
| 126 | 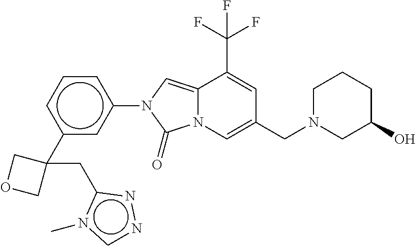 |
| 127 | 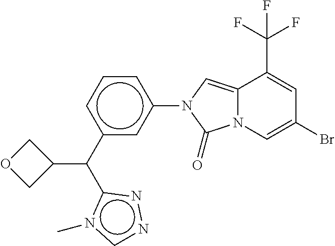 |
| 128 | 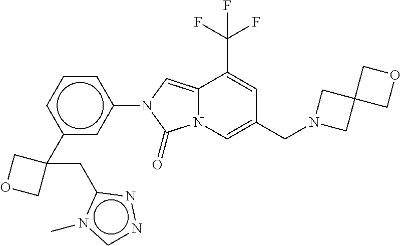 |
| 129 | 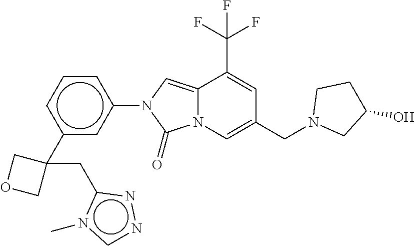 |
| 130 | 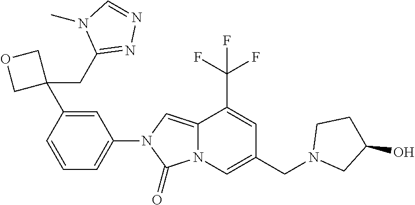 [SMILES: Cn1cnnc1CC1(c2cccc(-n3cc4c(C(F)(F)F)cc(CN5CC[C@@H](O)C5)cn4c3=O)c2)COC1] |
| 131 | 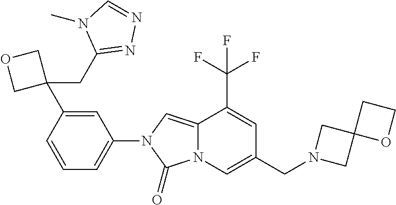 [SMILES: Cn1cnnc1CC1(c2cccc(-n3cc4c(C(F)(F)F)cc(CN5CC6(CCO6)C5)cn4c3=O)c2)COC1] |
| 132 | 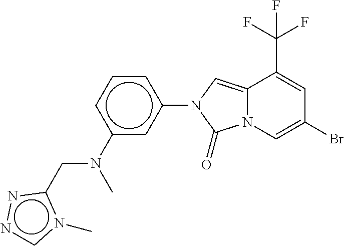 |
| 133 | 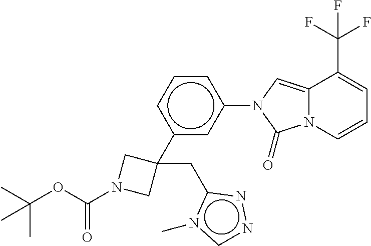 |
| 134 | 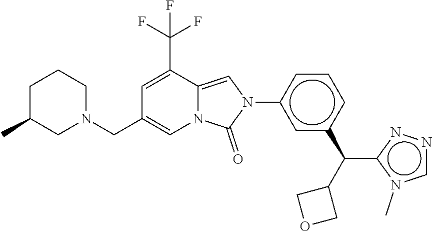 |
| 135 | 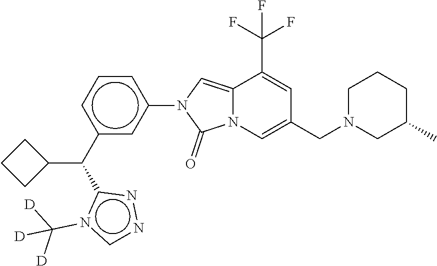 |
| 136 | 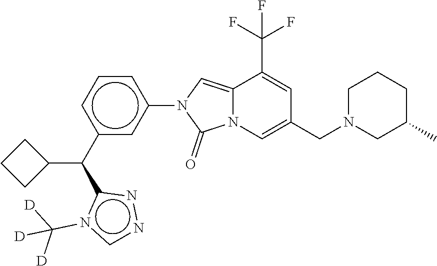 |
| 137 | 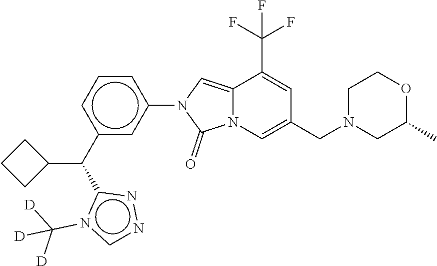 |
| 138 | 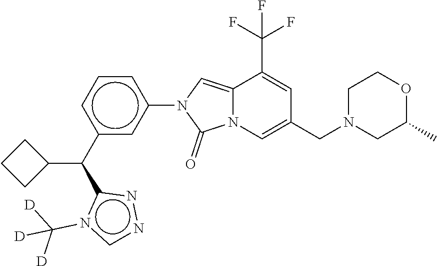 |
| 139 | 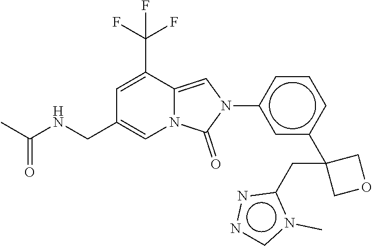 |
| 140 | 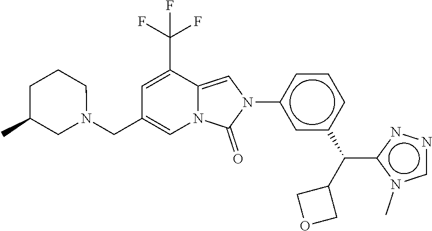 |
| 141 | 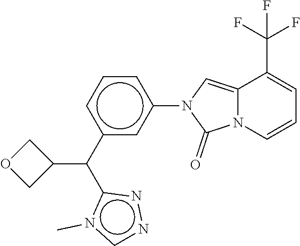 |
| 142 | 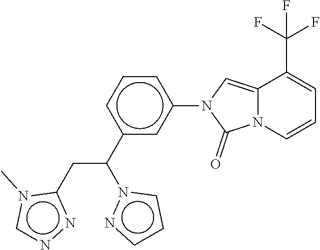 |
| 143 | 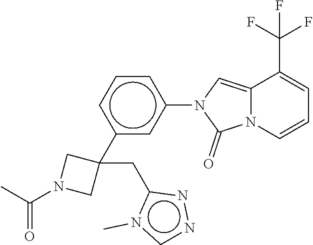 |
| 144 | 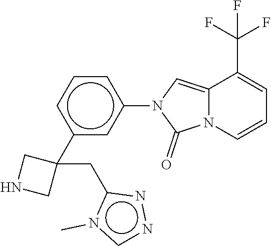 |
| 145 | 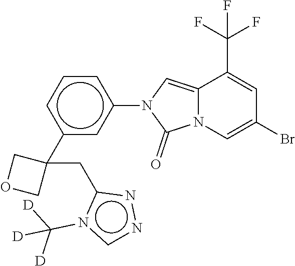 |
| 146 | 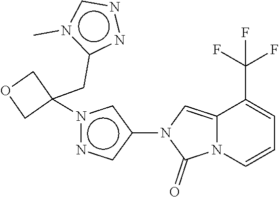 |
| 147 | 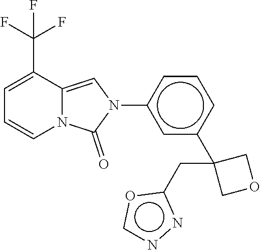 |
| 148 | 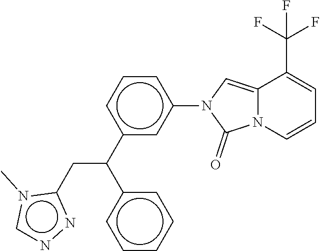 |
| 149 | 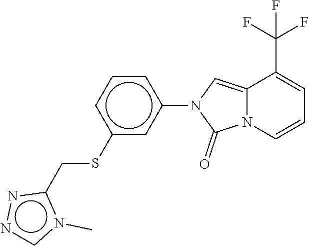 |
| 150 | 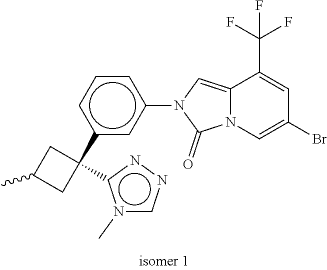 |
| 151 | 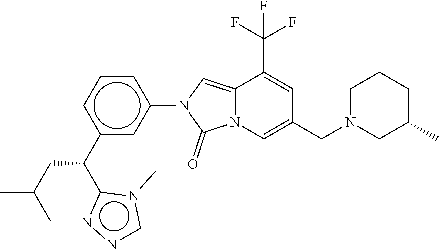 |
| 152 | 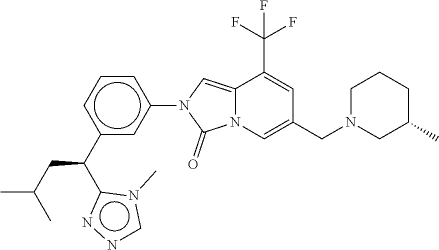 |
| 153 | 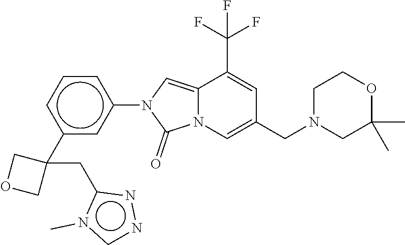 |
| 154 | 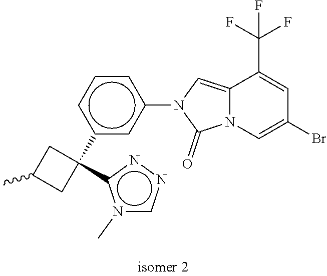 |
| 155 | 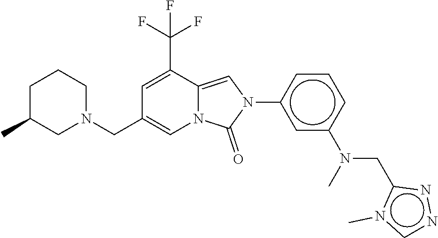 |
| 156 | 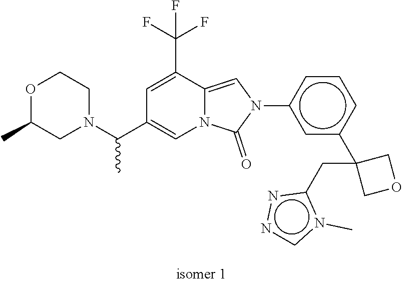 |
| 157 | 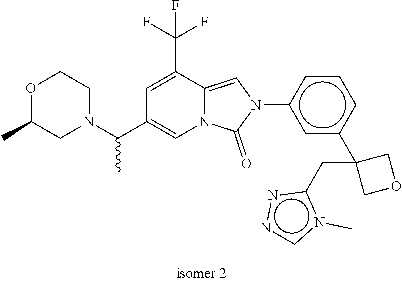 |
| 158 | 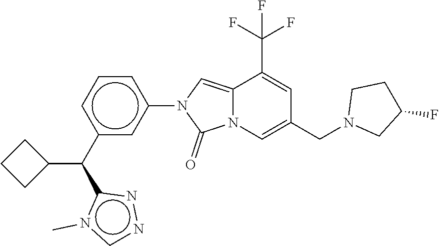 |
| 159 | 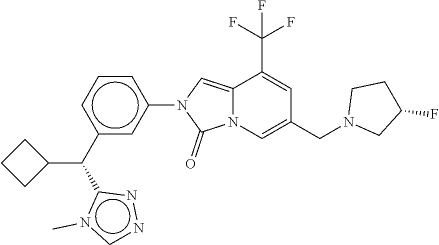 |
| 160 | 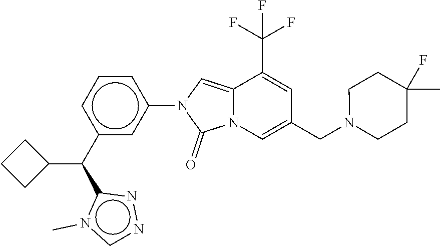 |
| 161 | 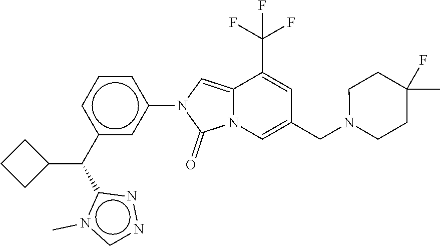 |
| 162 | 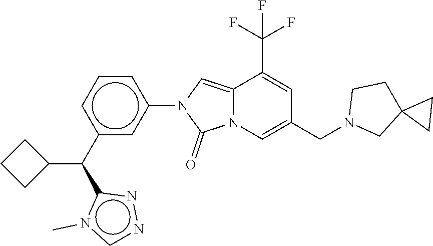 |
| 163 | 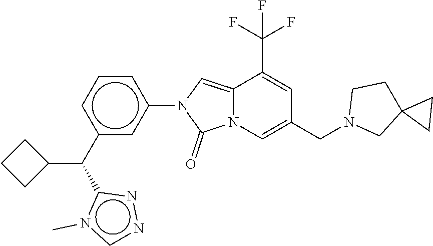 |
| 164 | 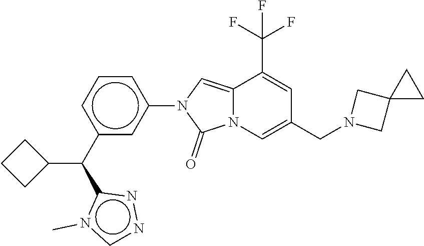 |
| 165 | 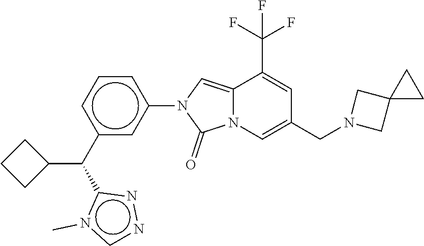 |
| 166 | 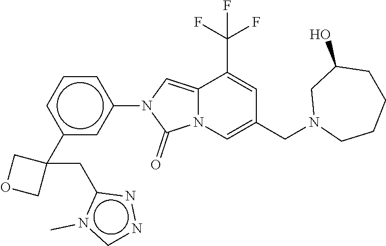 |
| 167 | 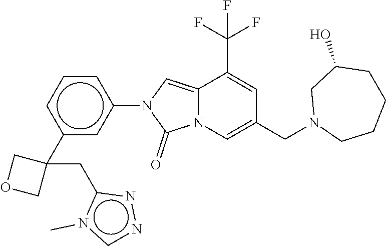 |
| 168 | 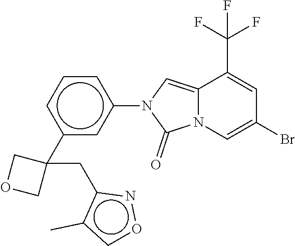 |
| 169 | 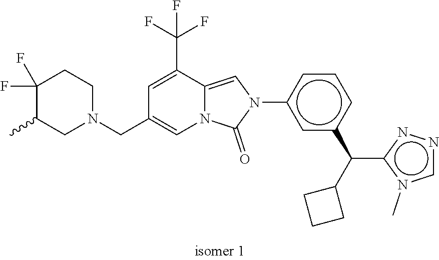 |
| 170 | 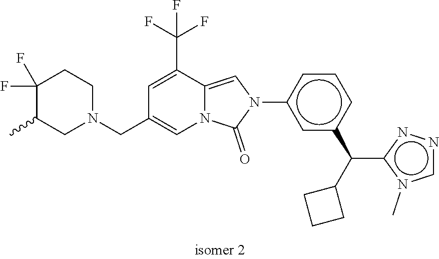 |
| 171 | 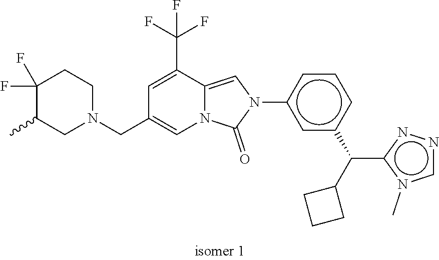 |
| 172 | 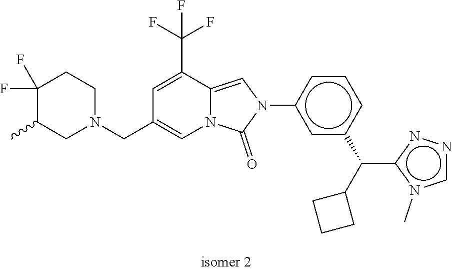 |
| 173 | 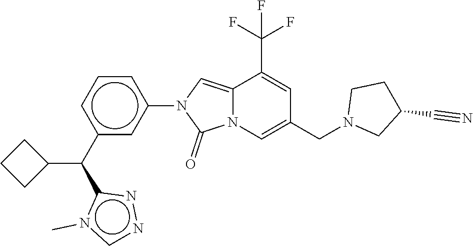 |
| 174 | 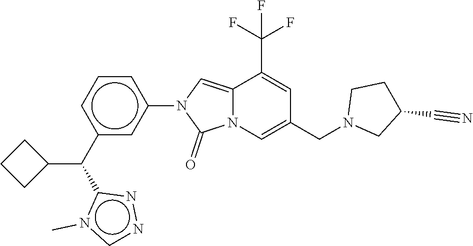 |
| 175 | 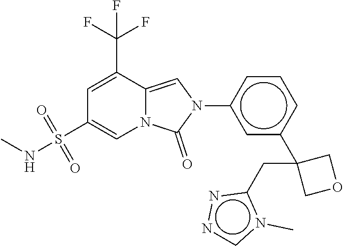 |
| 176 | 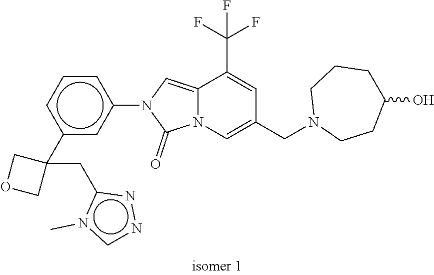 |
| 177 | 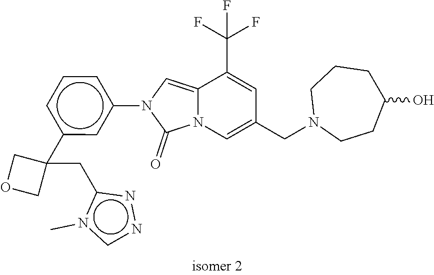 |
| 178 | 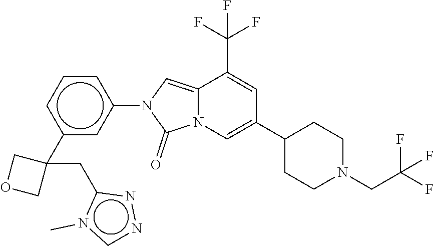 |
| 179 | 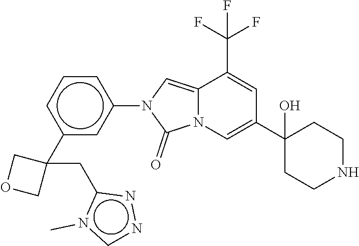 |
| 180 | 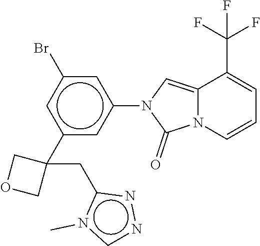 |
| 181 | 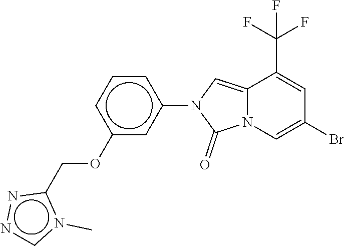 |
| 182 | 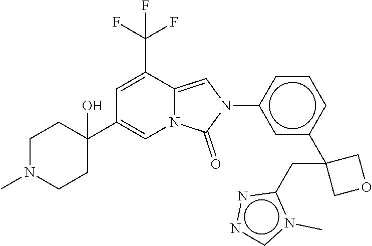 |
| 183 | 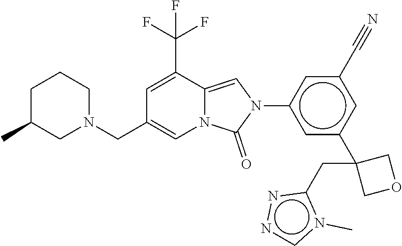 |
| 184 | 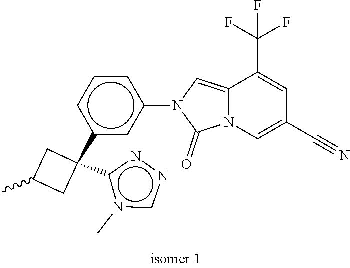 |
| 185 | 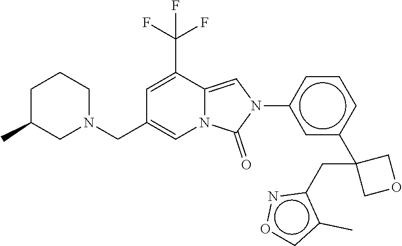 |
| 186 | 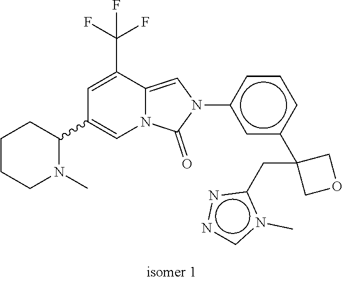 |
| 187 | 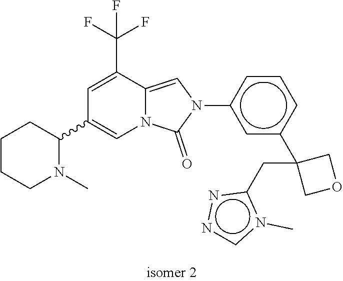 |
| 188 | 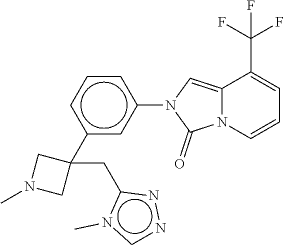 |
| 189 | 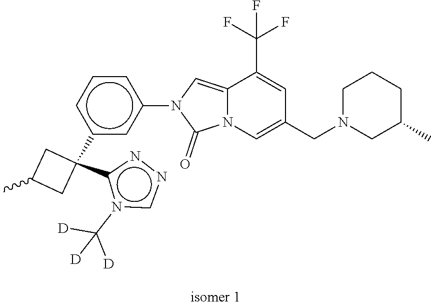 |
| 190 | 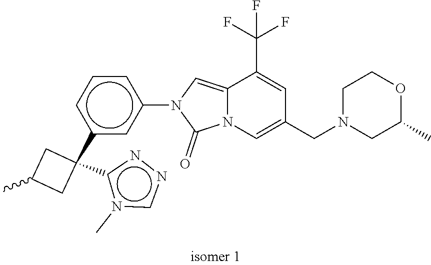 |
| 191 | 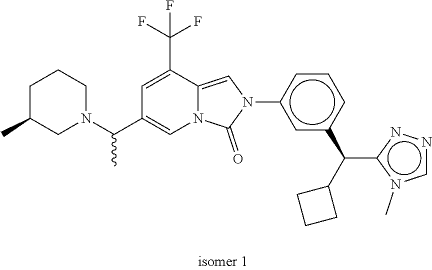 |
| 192 | 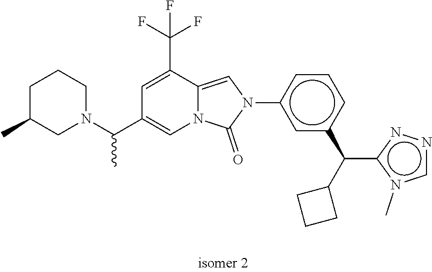 |
| 193 | 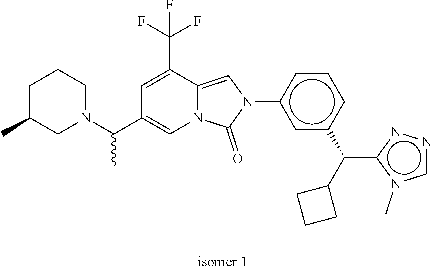 |
| 194 | 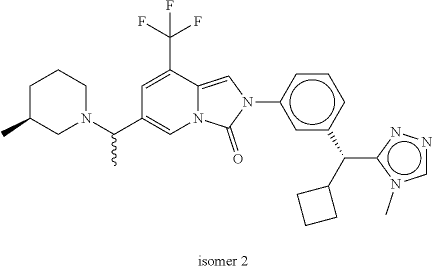 |
| 195 | 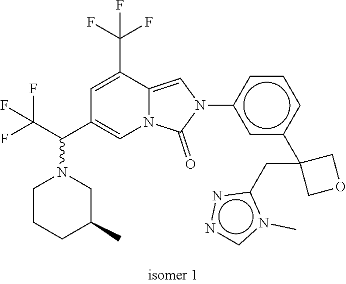 |
| 196 | 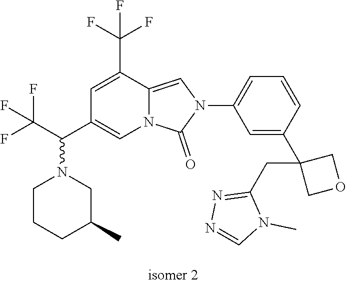 [SMILES: C[C@H]1CCCN(C(c2cc(C(F)(F)F)c3cn(-c4cccc(C5(Cc6nncn6C)COC5)c4)c(=O)n3c2)C(F)(F)F)C1] |
| 197 | 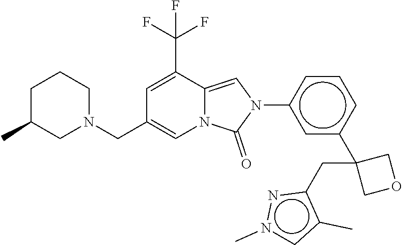 |
| 198 | 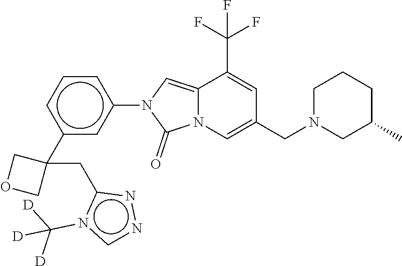 |
| 199 | 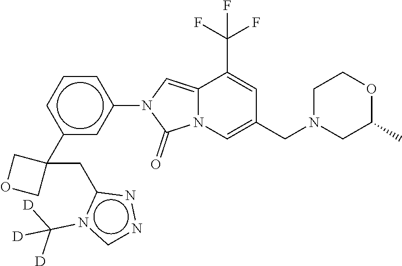 |
| 200 | 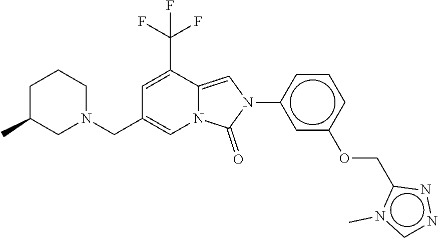 |
| 201 | 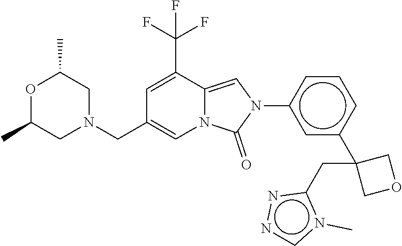 |
| 202 | 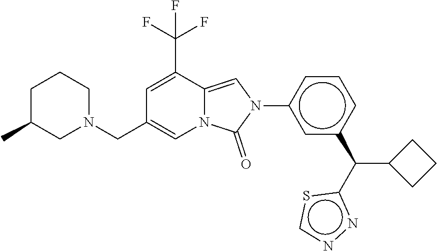 |
| 203 | 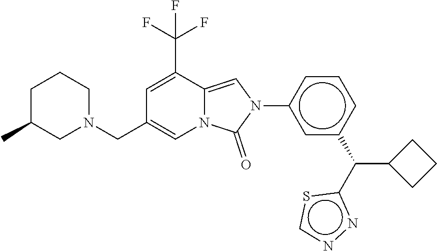 |
| 204 | 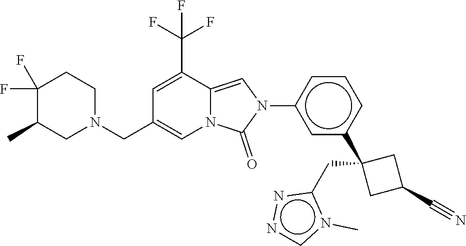 |
| 205 | 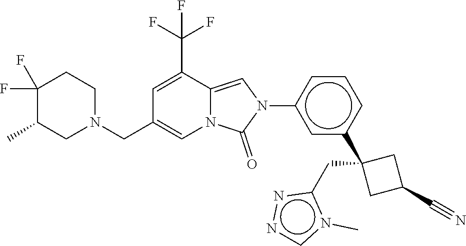 |
| 206 | 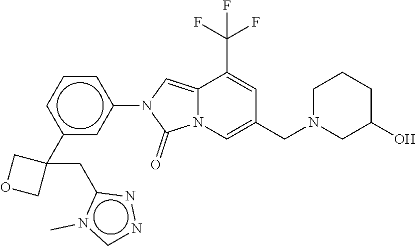 |
| 207 | 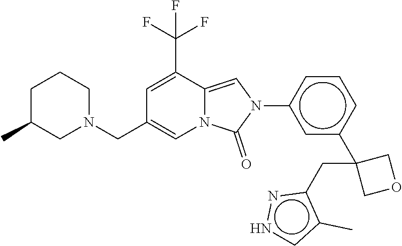 |
| 208 | 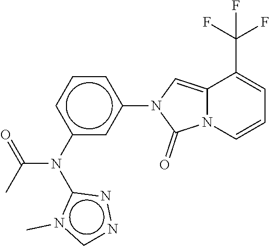 |
| 209 | 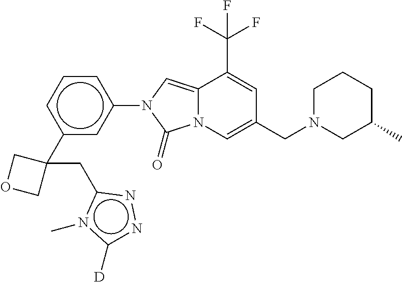 |
| 210 | 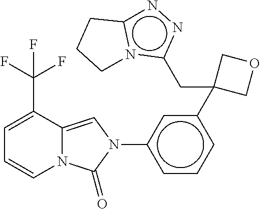 |
| 211 | 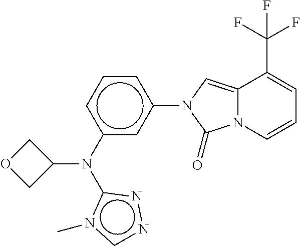 |
| 212 | 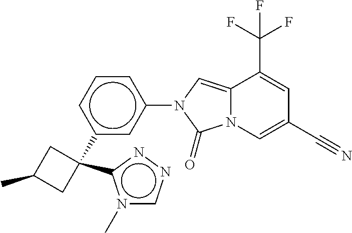 |
| 213 | 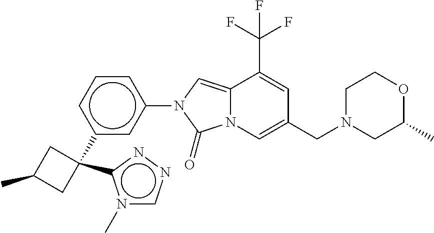 |
| 214 | 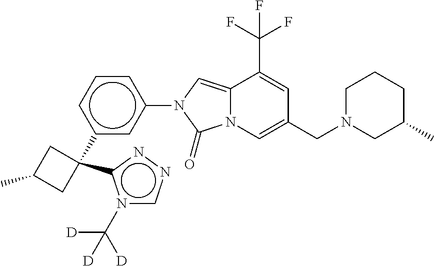 |
| 215 | 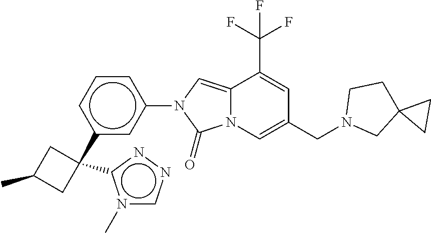 |
| 216 | 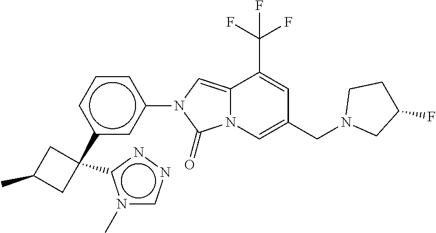 |
| 217 | 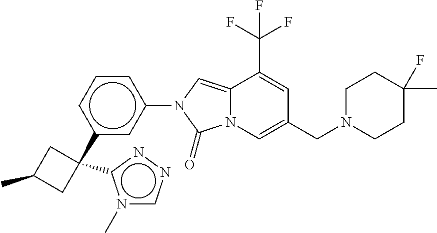 |
| 218 | 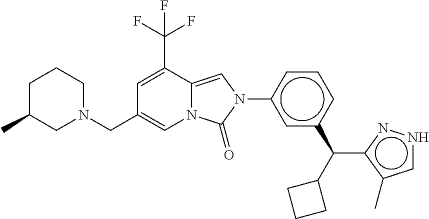 |
| 219 | 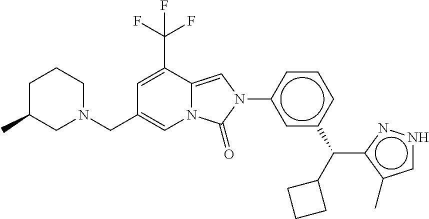 |
| 220 | 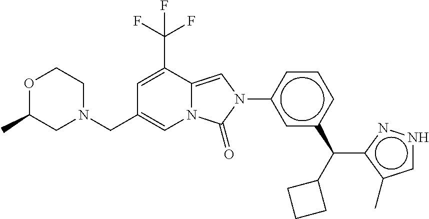 |
| 221 | 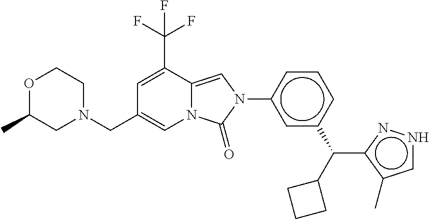 |
| 222 | 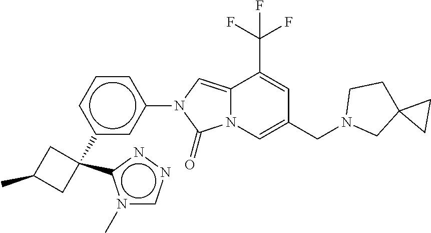 |
| 223 | 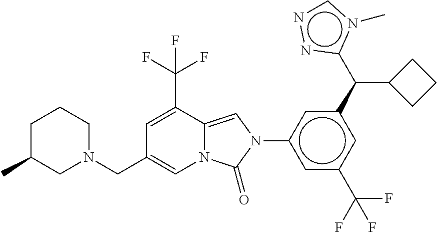 |
| 224 | 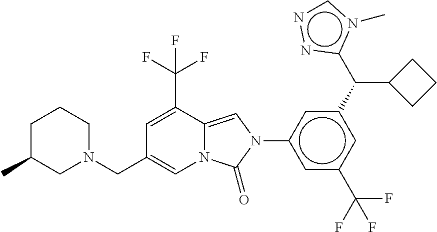 |
| 225 | 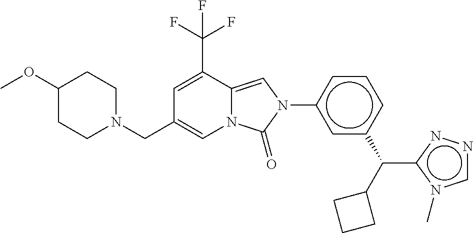 |
| 226 | 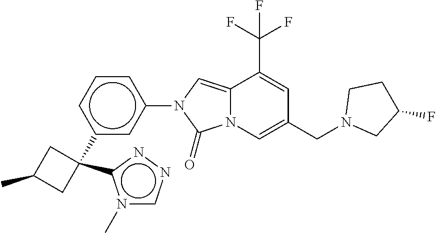 |
| 227 | 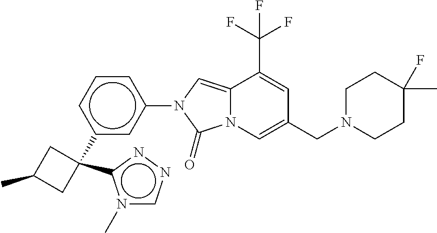 |
| 228 | 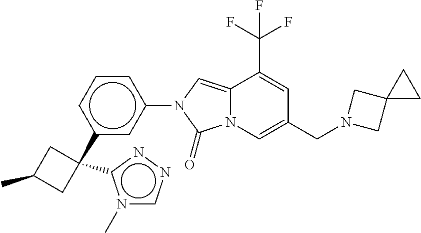 |
| 229 | 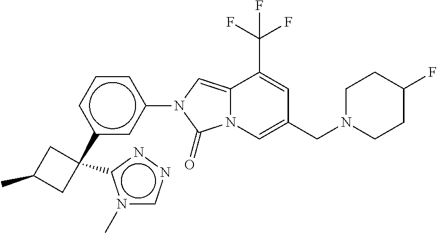 |
| 230 | 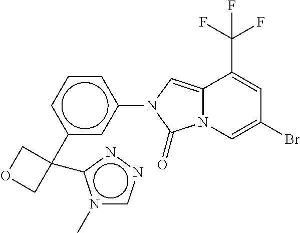 |
| 231 | 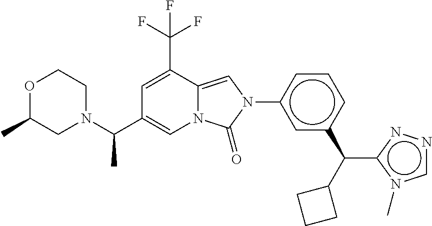 |
| 232 | 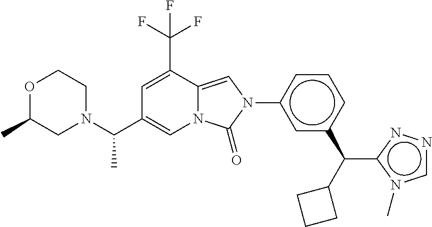 |
| 233 | 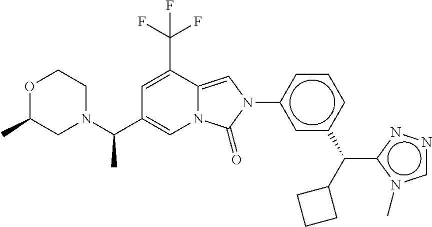 |
| 234 | 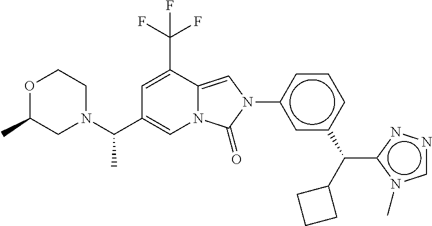 |
| 235 | 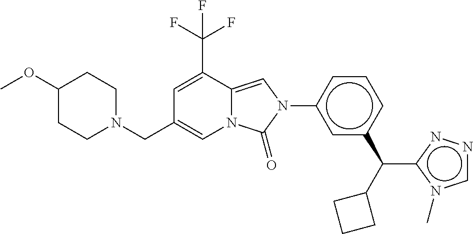 |
| 236 | 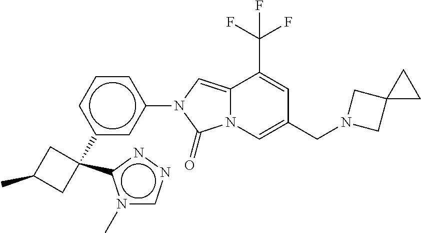 |
| 237 | 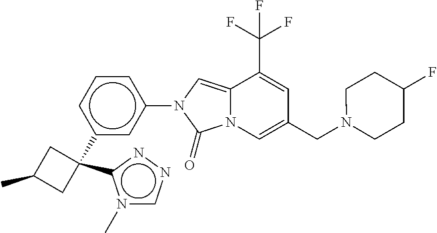 |
| 238 | 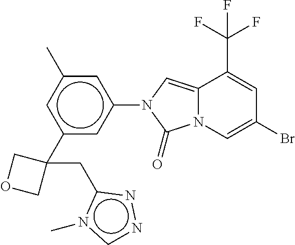 |
| 239 | 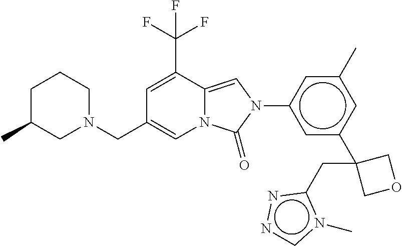 |
| 240 | 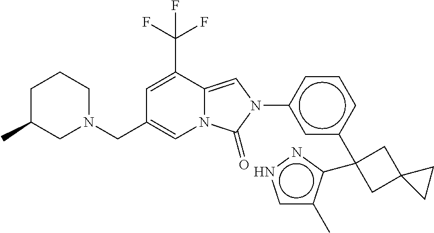 |
| 241 | 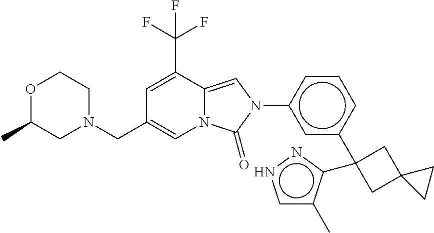 |
| 242 | 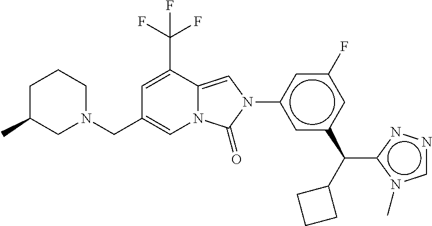 |
| 243 | 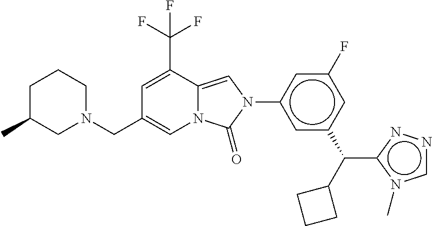 |
| 244 | 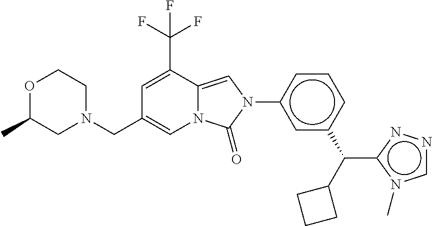 |
| 245 | 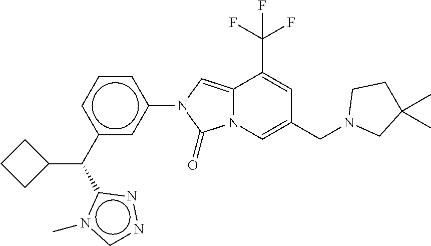 |
| 246 | 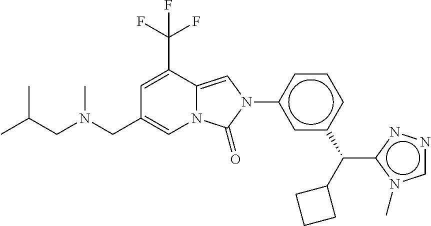 |
| 247 | 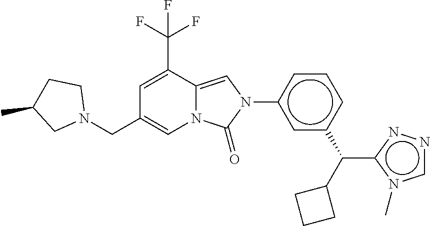 |
| 248 | 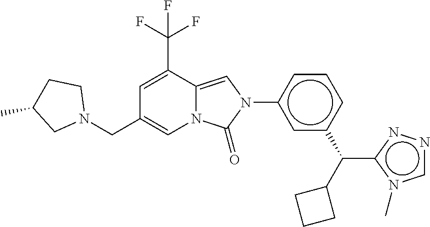 |
| 249 | 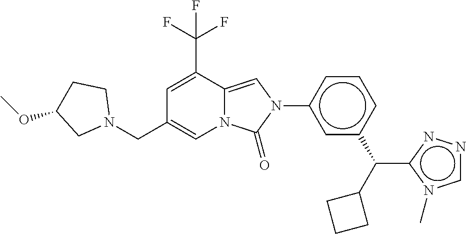 |
| 250 | 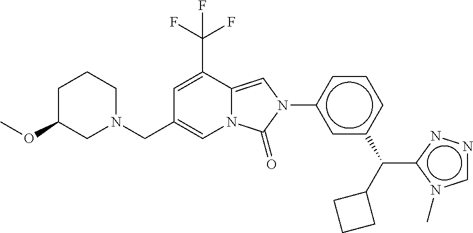 |
| 251 | 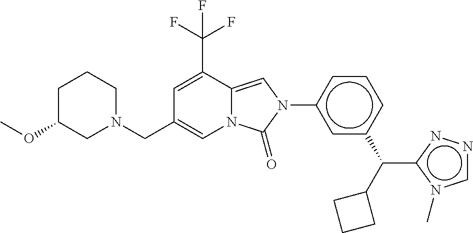 |
| 252 | 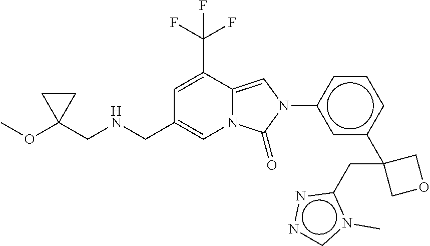 |
| 253 | 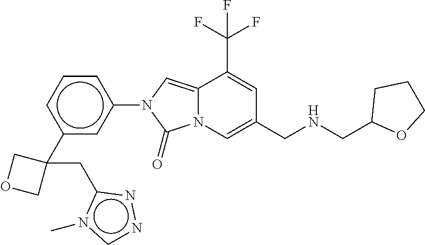 |
| 254 | 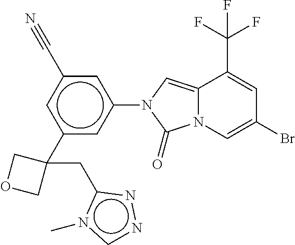 |
| 255 | 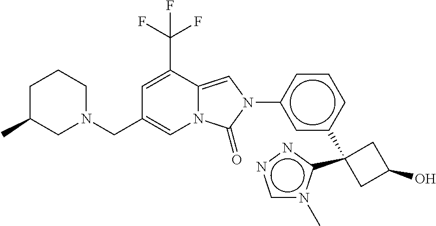 |
| 256 | 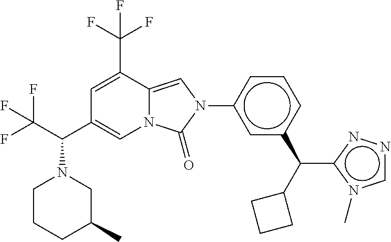 |
| 257 | 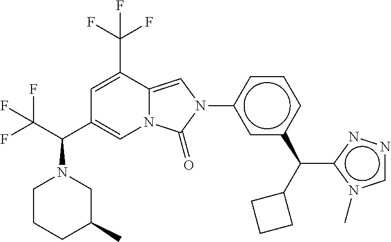 |
| 258 | 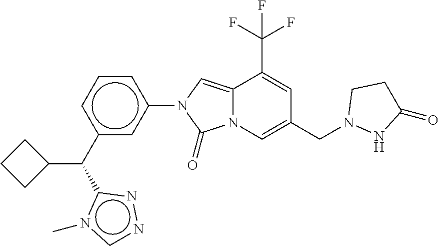 |
| 259 | 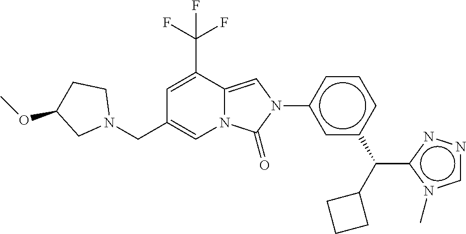 |
| 260 | 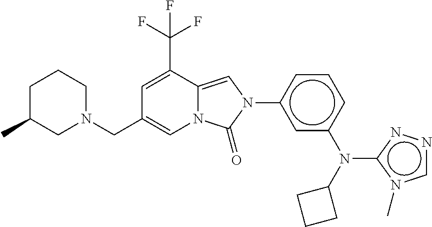 |
| 261 | 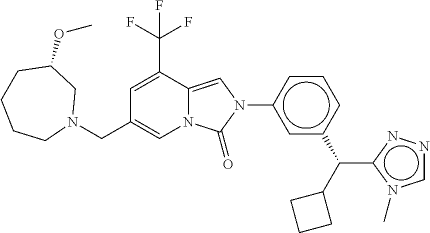 |
| 262 | 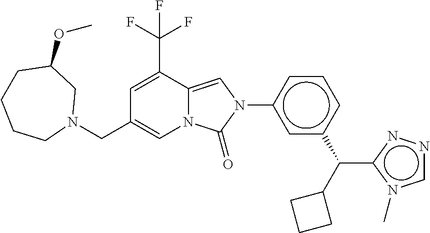 |
| 263 | 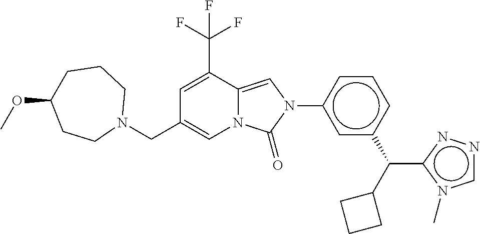 |
| 264 | 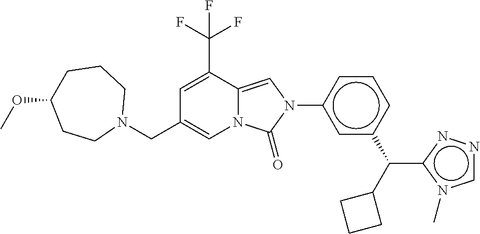 |
| 265 | 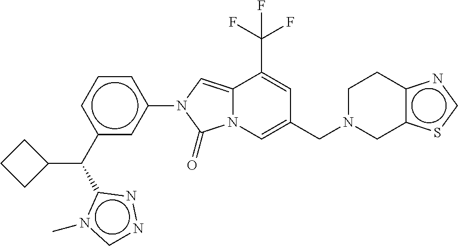 |
| 266 | 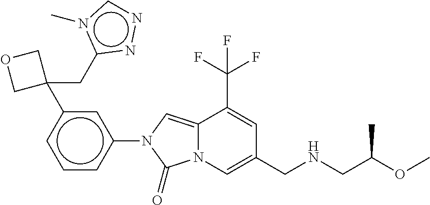 |
| 267 | 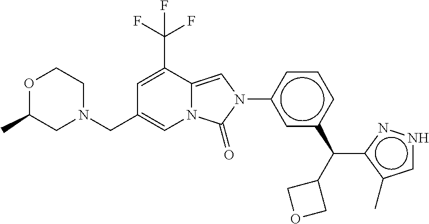 |
| 268 | 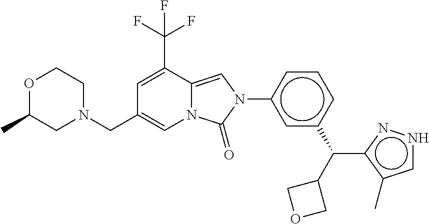 |
| 269 | 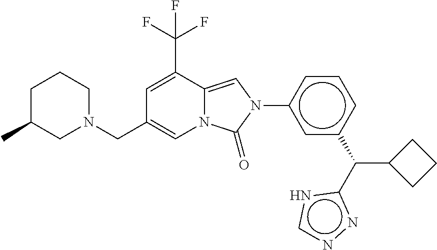 |
| 270 | 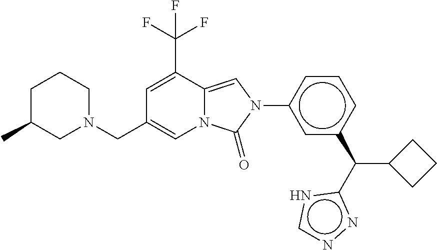 |
| 271 | 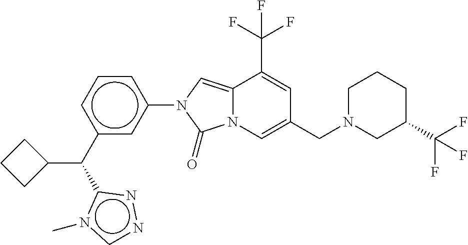 |
| 272 | 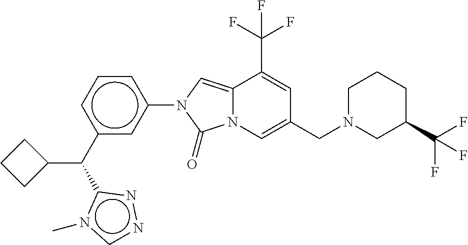 |
| 273 | 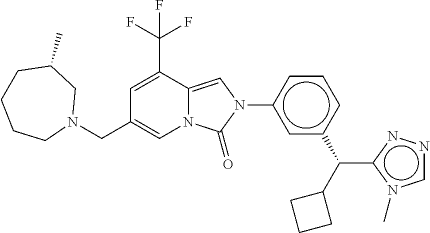 |
| 274 | 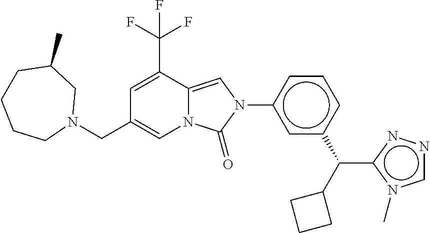 |
| 275 | 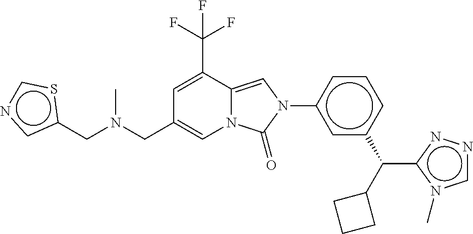 |
| 276 | 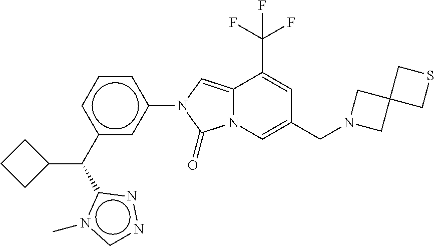 |
| 277 | 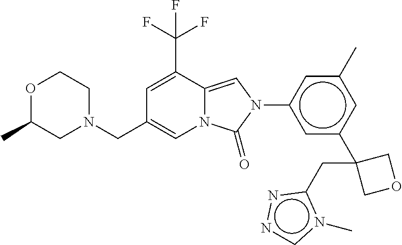 |
| 278_p1 | 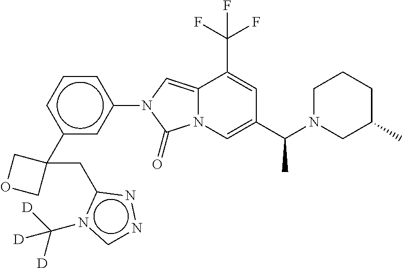 |
| 278_p2 | 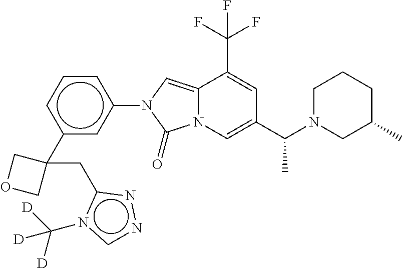 |
| 279 | 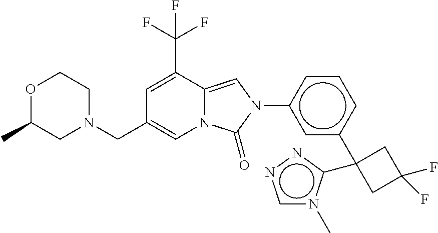 |
| 280 | 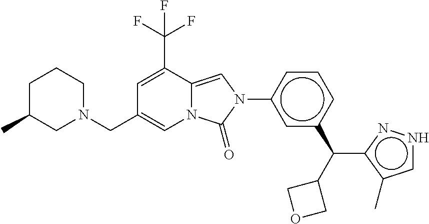 |
| 281 | 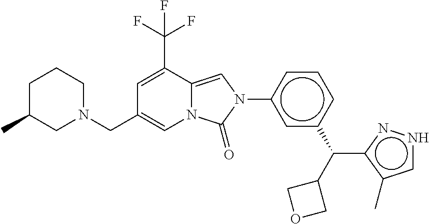 |
| 282 | 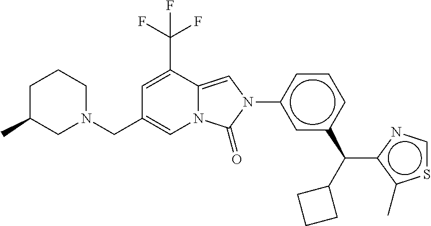 |
| 283 | 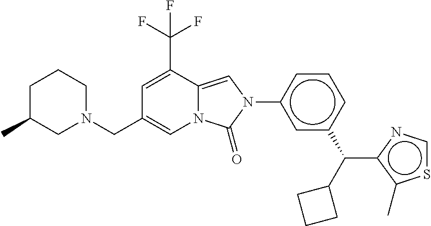 |
| 284 | 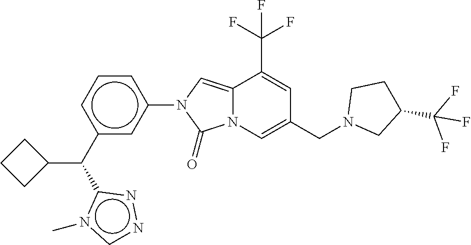 |
| 285 | 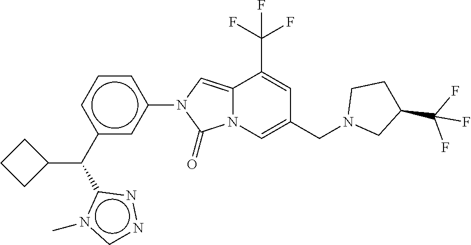 |
| 286 | 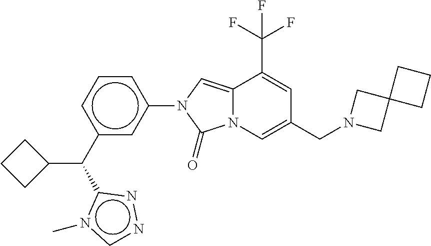 |
| 287_P1 | 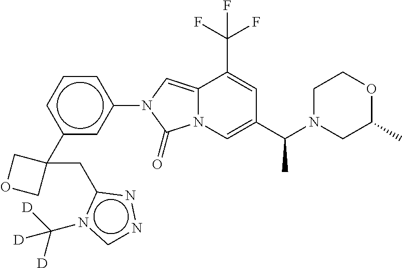 |
| 287_P2 | 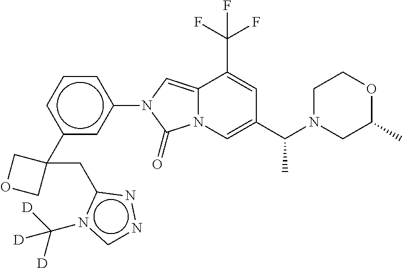 |
| 288 | 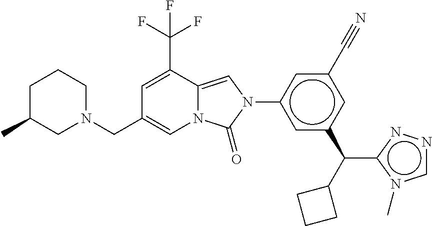 |
| 289 | 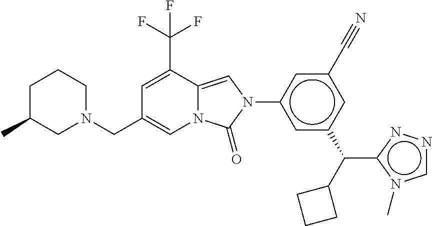 |
| 290 | 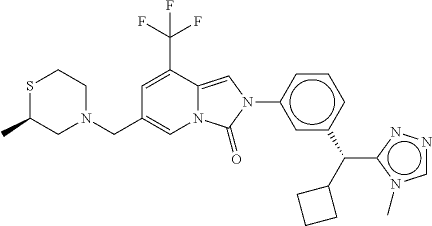 |
| 291 | 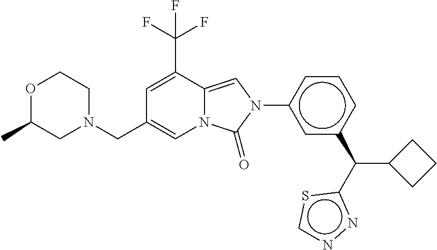 |
| 292 | 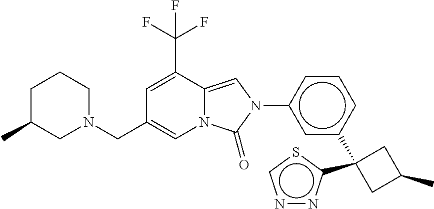 |
| 293 | 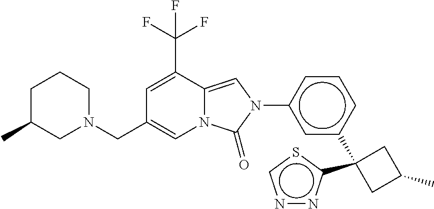 |
| 294 | 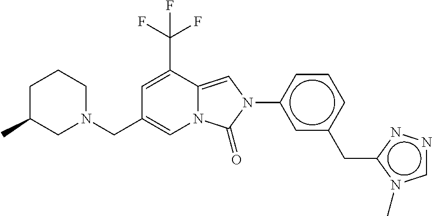 |
| 295 | 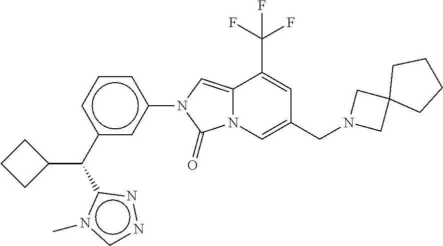 |
| 296 | 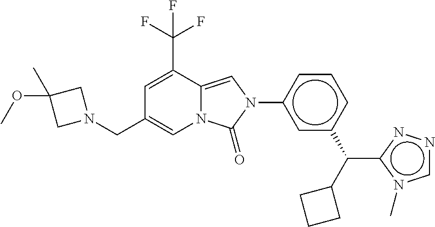 |
| 297 | 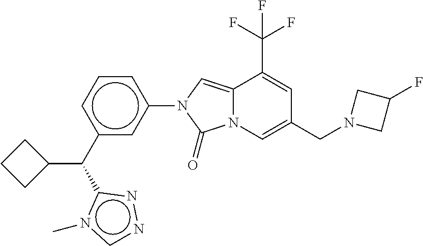 |
| 298 | 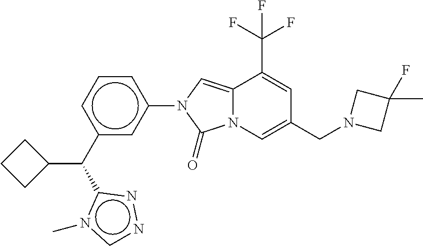 |
| 299 | 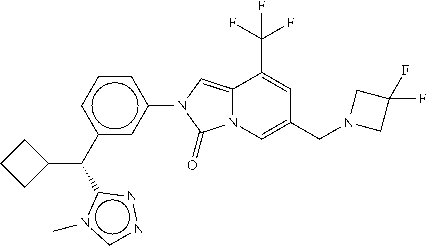 |
| 300 | 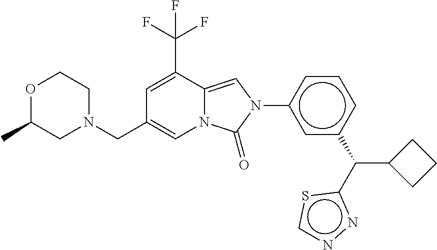 |
| 301 | 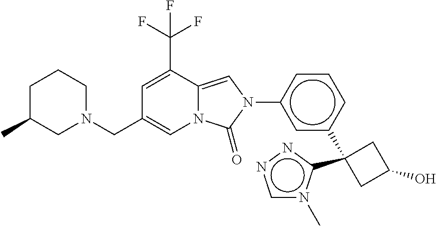 |
| 302 | 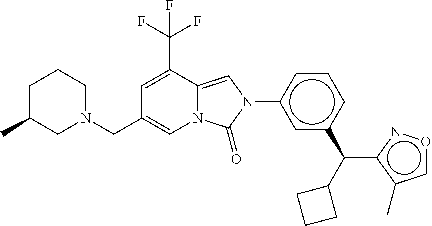 |
| 303 | 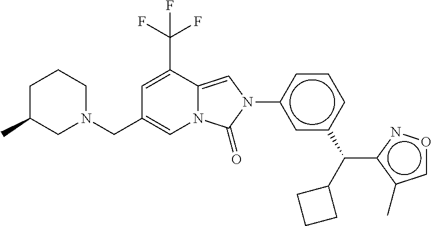 |
| 304 | 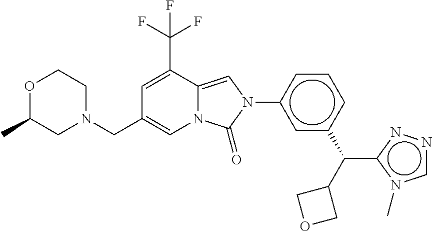 |
| 305 | 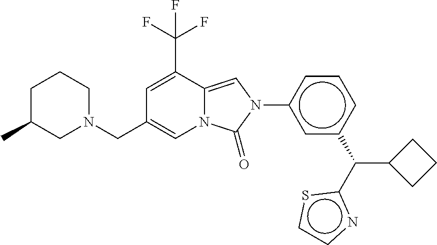 |
| 306 | 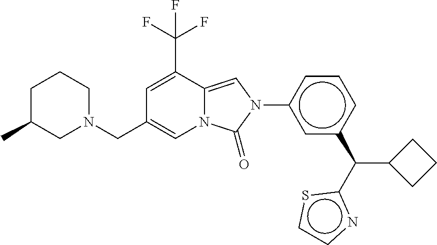 |
| 307 | 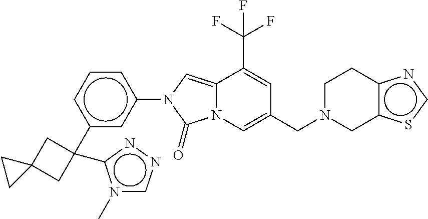 |
| 308 | 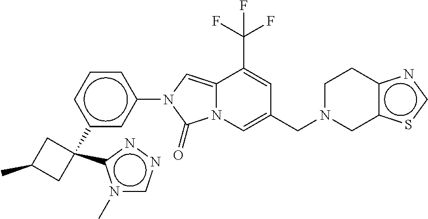 |
| 309 | 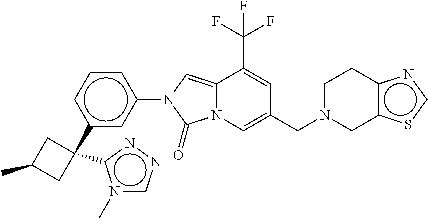 |
| 310 | 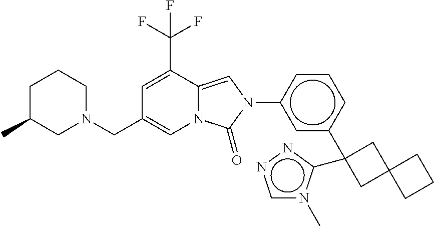 |
| 311 | 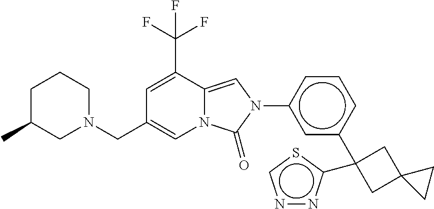 |
| 312 | 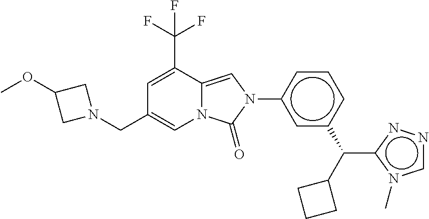 |
| 313 | 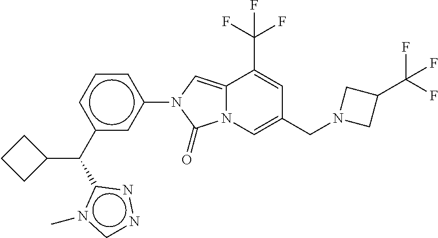 |
| 314 | 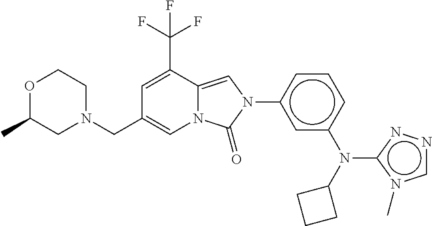 |
| 315 | 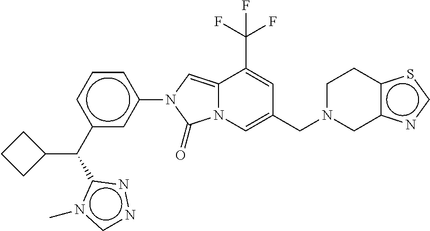 |
| 316 | 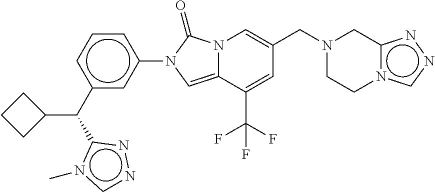 |
| 317 | 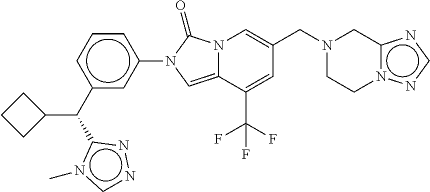 |
| 318 | 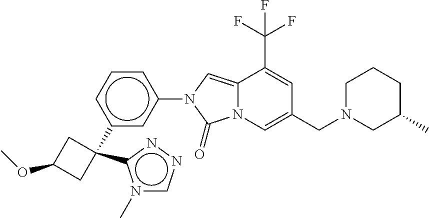 |
| 319 | 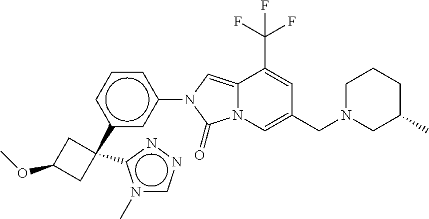 |
| 329 | 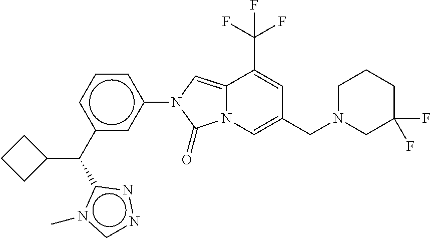 |
| 321 | 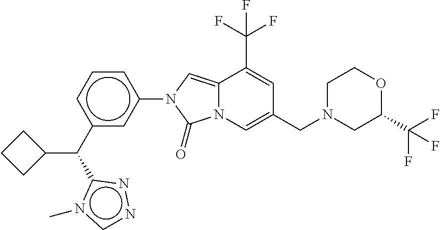 |
| 322 | 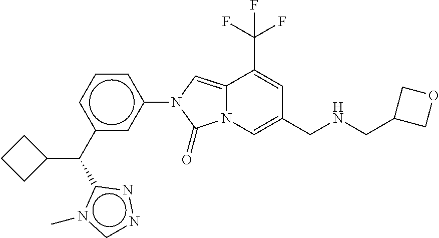 |
| 323 | 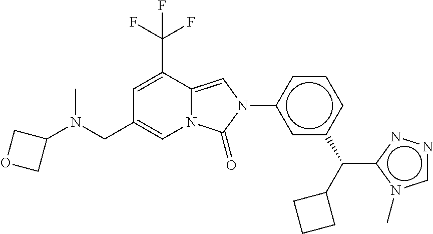 |
| 324 | 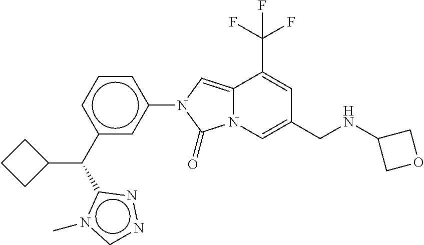 |
| 325 | 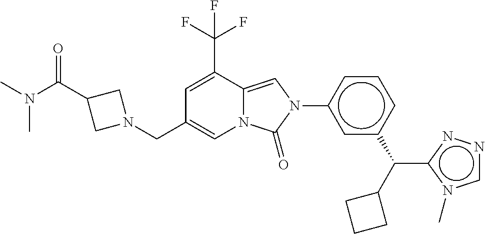 |
| 326 | 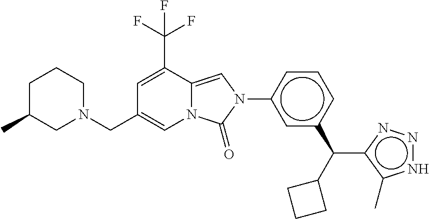 |
| 327 | 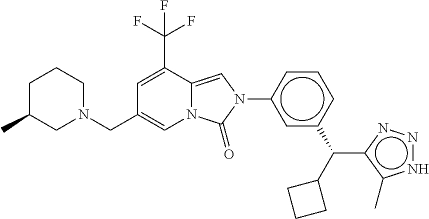 |
| 328 | 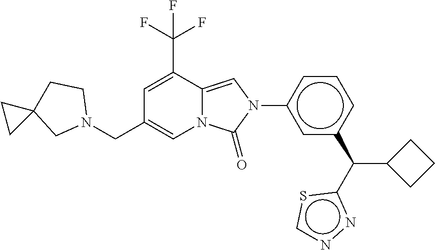 |
| 329 | 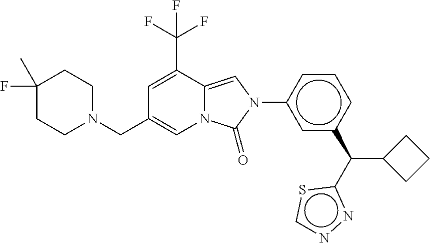 |
| 330 | 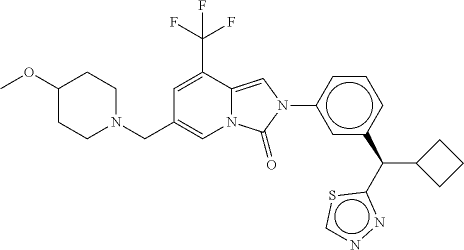 |
| 331 | 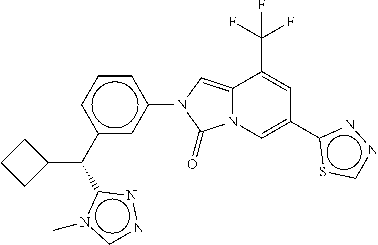 |
| 332_p1 | 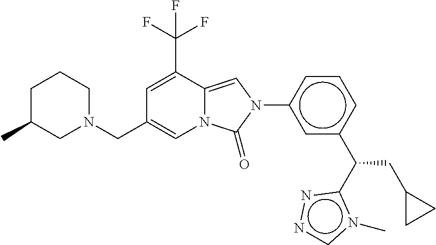 |
| 332-p2 | 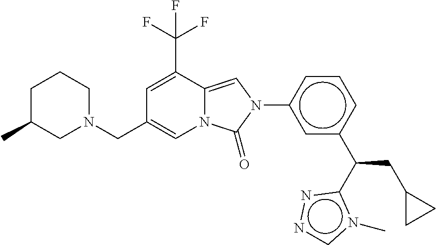 |
| 333 | 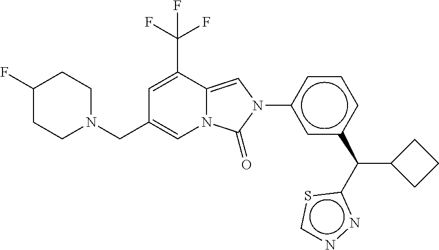 |
| 334 | 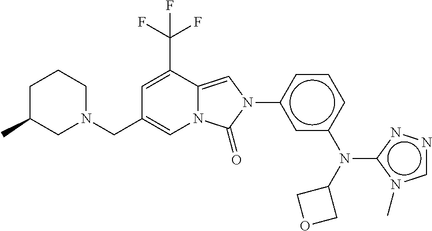 |
| 335 | 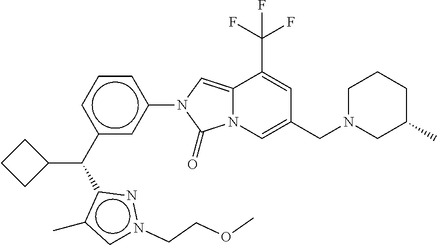 |
| 336 | 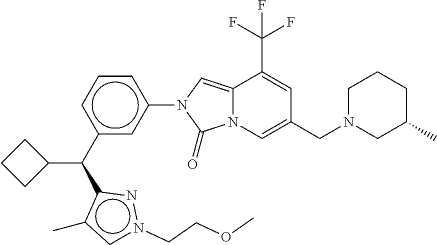 |
| 337 | 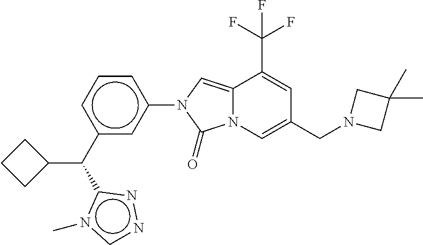 |
| 338 | 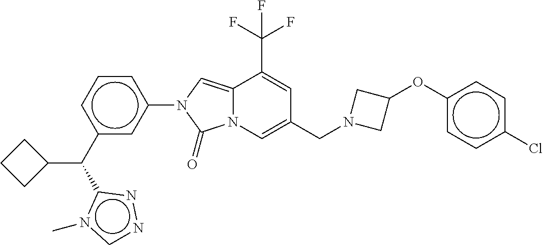 |
| 339_P1 | 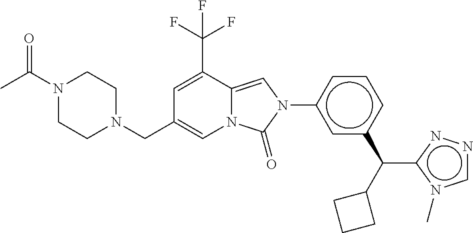 |
| 339_P2 | 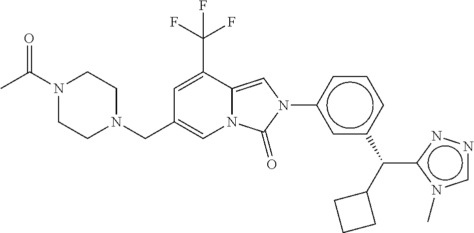 |
| 340_P1 | 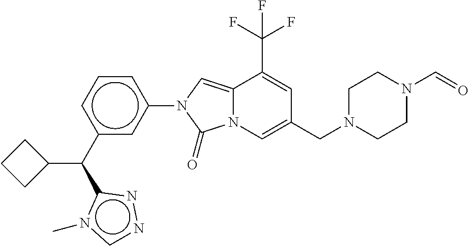 |
| 340_P2 | 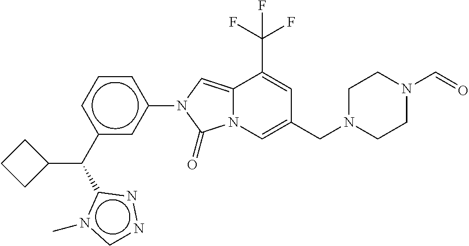 |
| 341 | 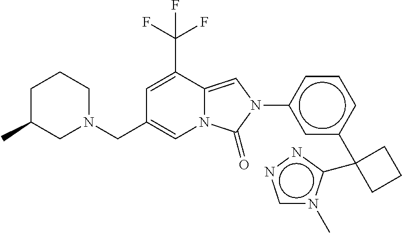 |
| 342 | 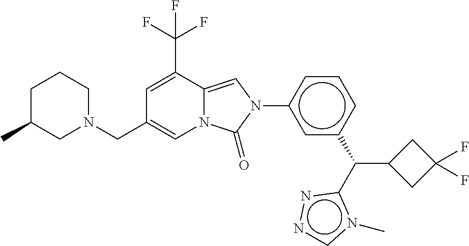 |
| 343 | 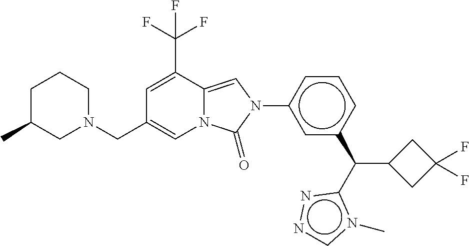 |
| 344 | 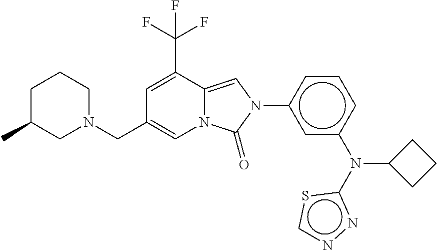 |
| 345 | 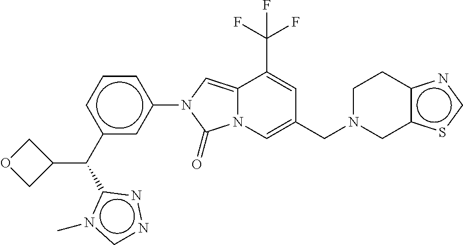 |
| 346 | 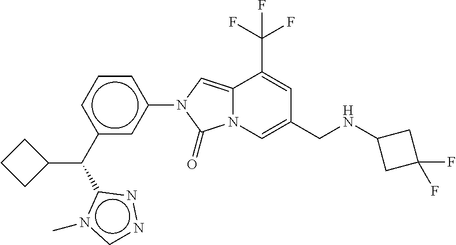 |
| 347 | 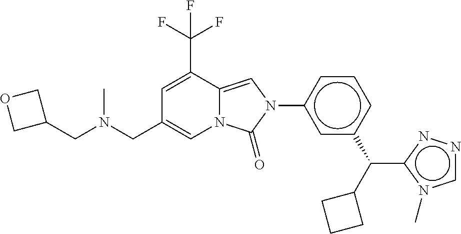 |
| 348 | 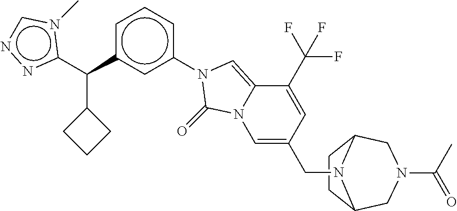 |
| 349 | 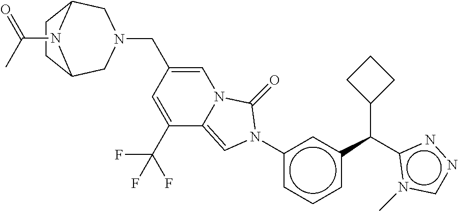 |
| 350 | 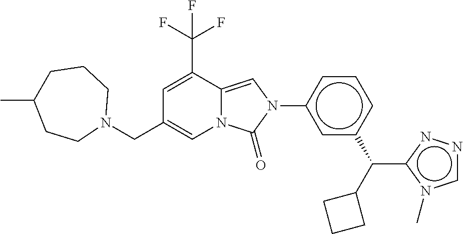 |
| 351_P1 | 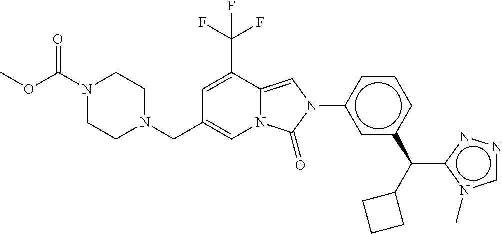 |
| 351_P2 | 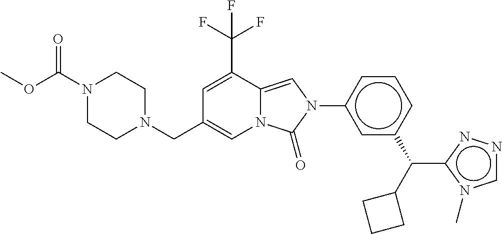 |
| 352_P1 | 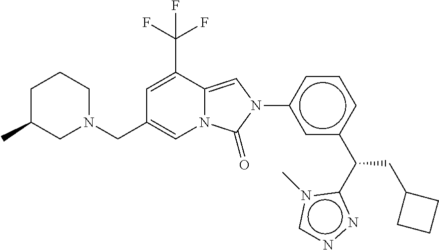 |
| 352_P2 | 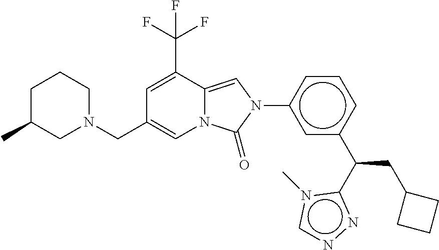 |
| 353 | 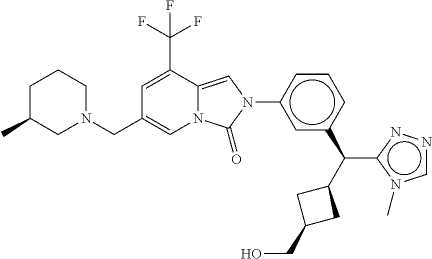 |
| 354 | 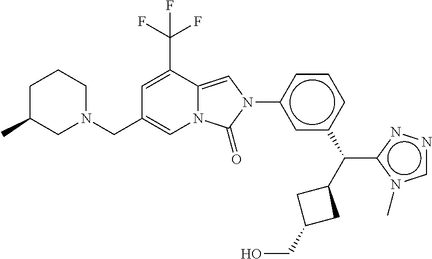 |
| 355 | 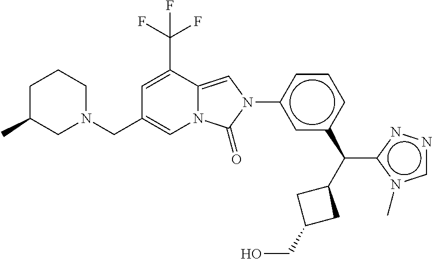 |
| 356 | 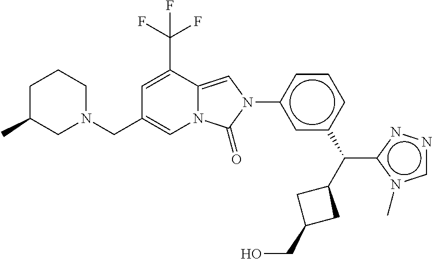 |
| 357 | 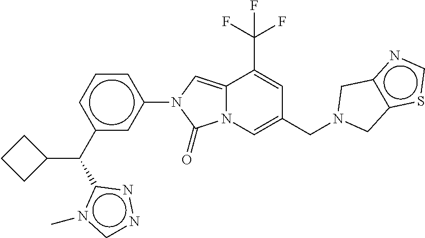 |
| 358 | 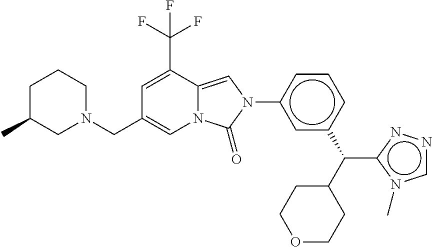 |
| 359 | 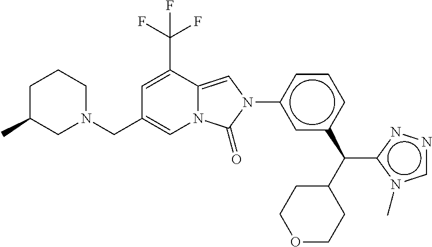 |
| 360 | 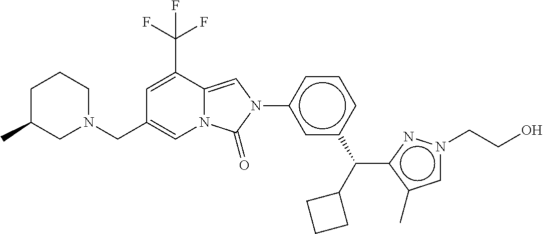 |
| 361 | 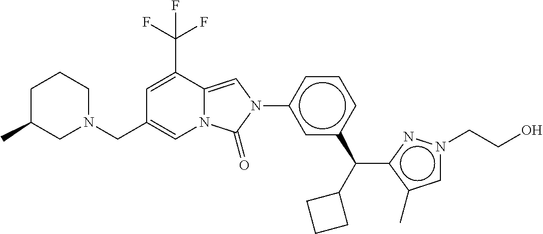 |
| 362_P1 | 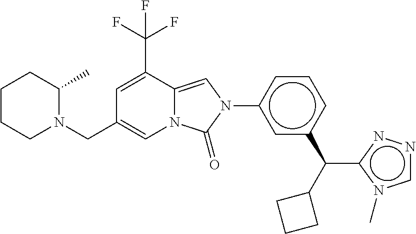 |
| 362_P2 | 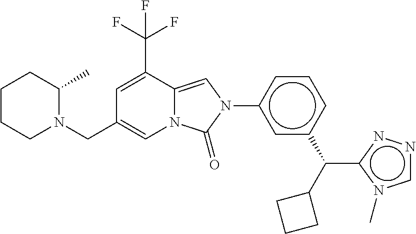 |
| 363_P1 | 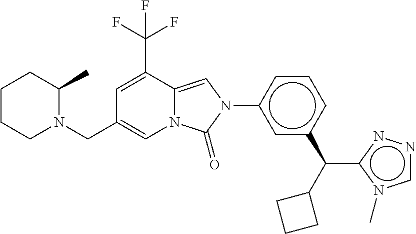 |
| 363_P2 | 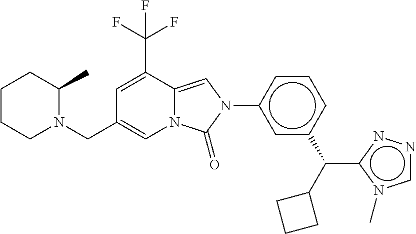 |
| 364_P1 | 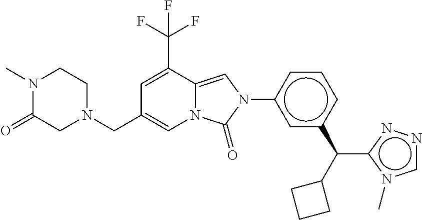 |
| 364_P2 | 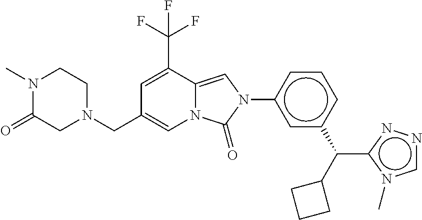 |
| 365 | 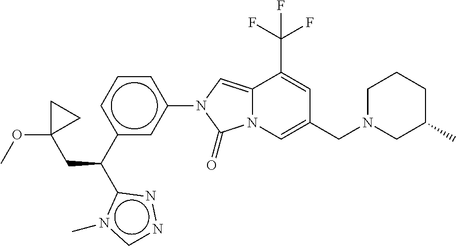 |
| 366 | 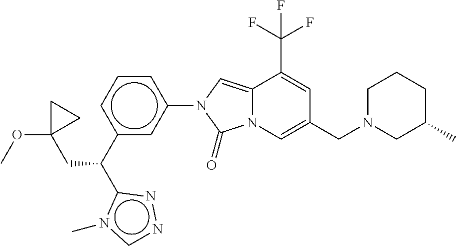 |
| 367 | 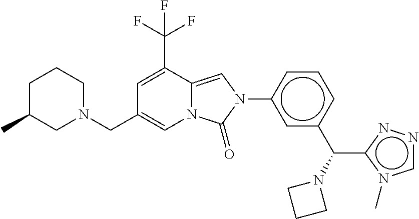 |
| 368 | 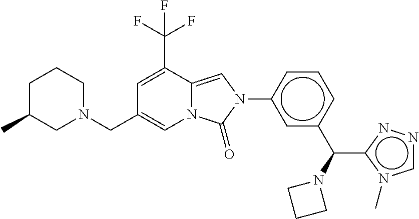 |
| 369 | 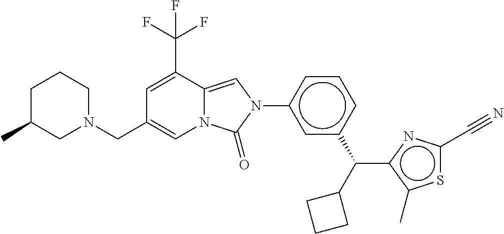 |
| 370 | 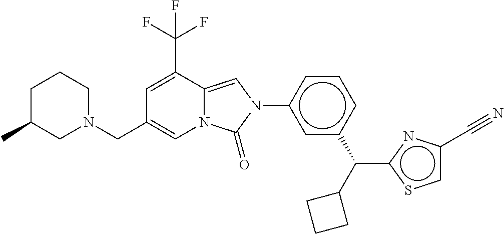 |
| 371 | 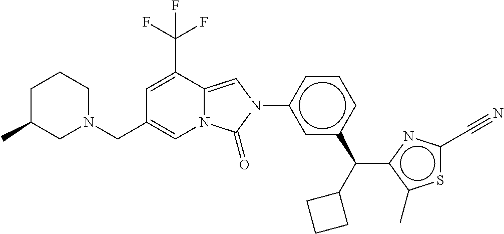 |
| 372 | 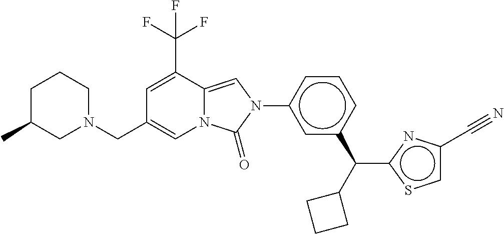 |
| 373 | 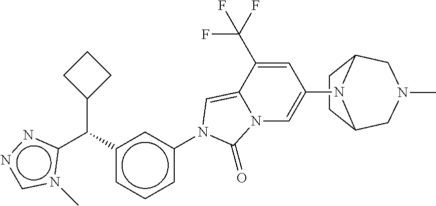 |
| 374 | 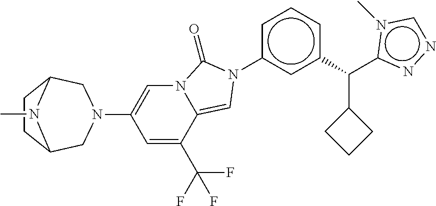 |
| 375 | 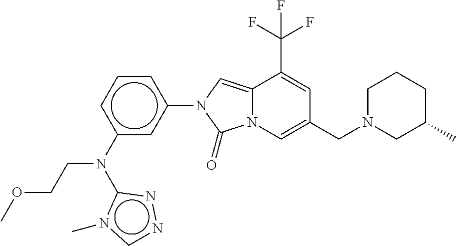 |
| 376 | 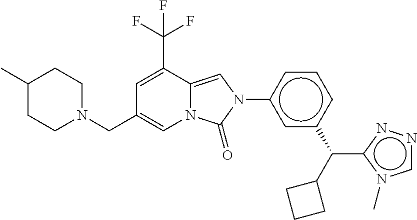 |
| 377 | 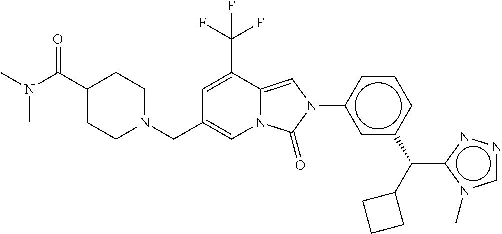 |
| 378 | 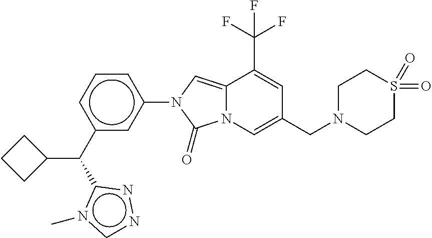 |
| 379 | 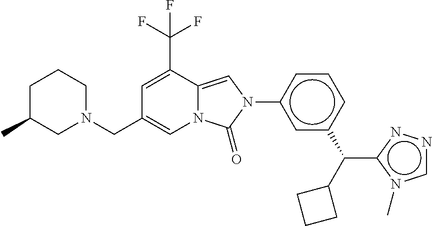 |
| 380_P1 | 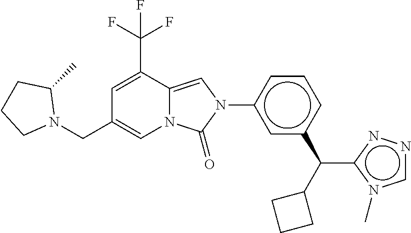 |
| 380_P2 | 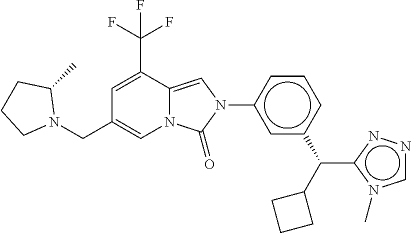 |
| 381_P1 | 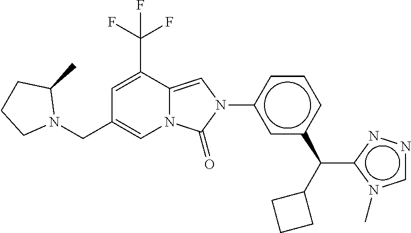 |
| 381_P2 | 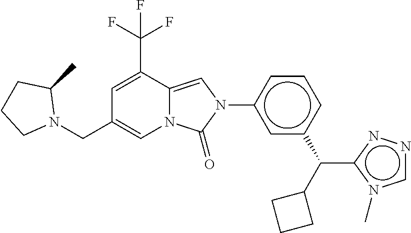 |
| | 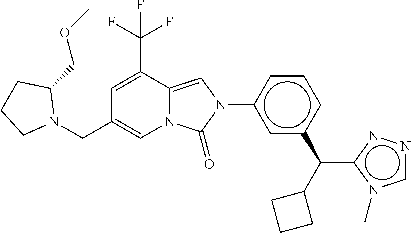 |
| | 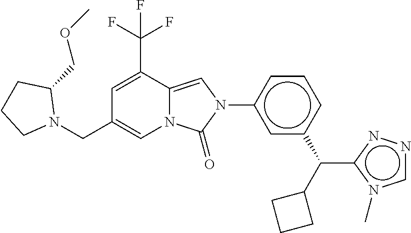 |
| 382_P1 | 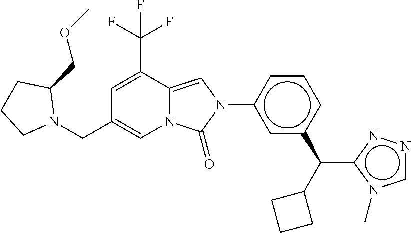 |
| 382_P2 | 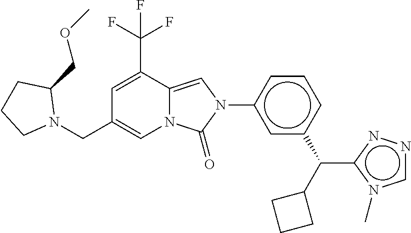 |
| 383 | 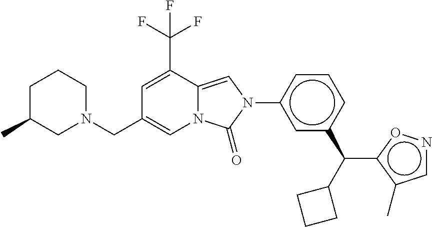 |
| 384 | 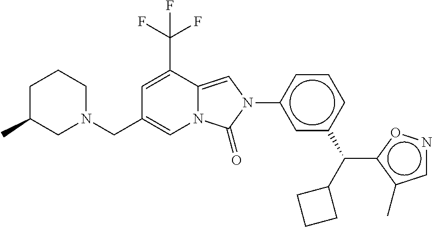 |
| 385 | 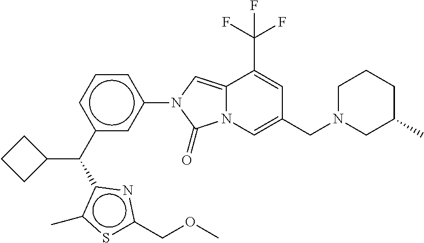 |
| 386 | 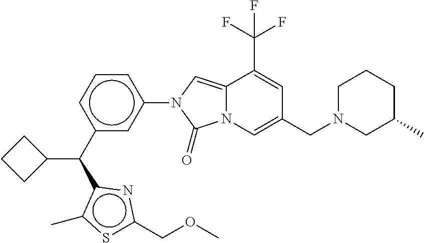 |
| 387 | 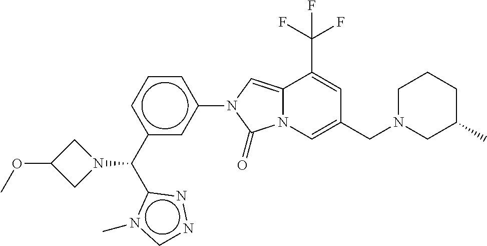 |
| 388 | 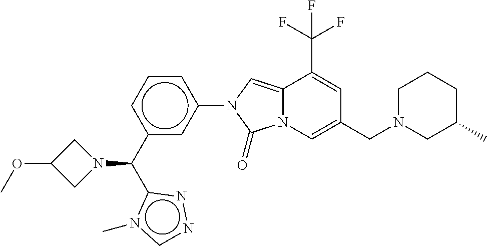 |
| 389 | 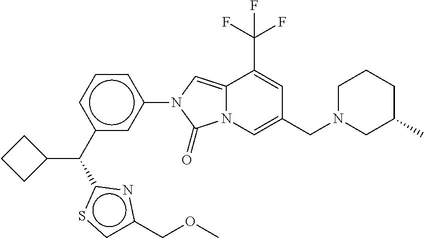 |
| 390 | 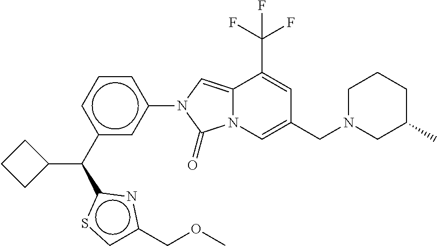 |
| 391 | 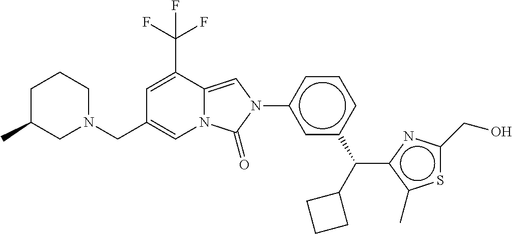 |
| 392 | 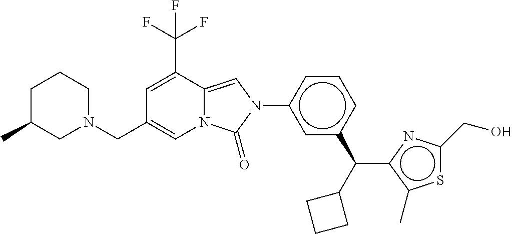 |
| 393 | 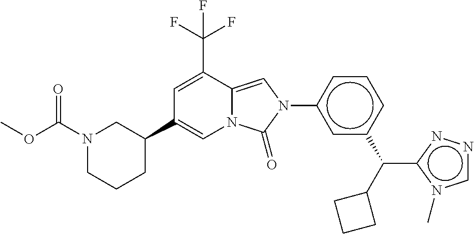 |
| 394 | 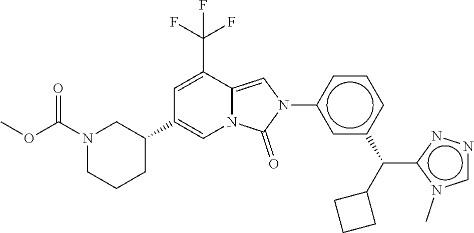 |
| 395 | 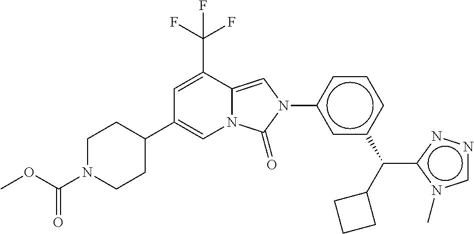 |
| 396 | 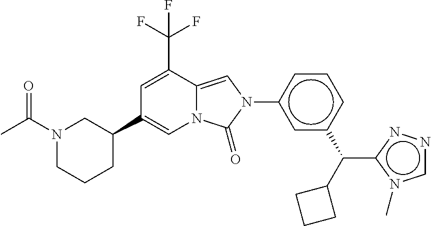 |
| 397 | 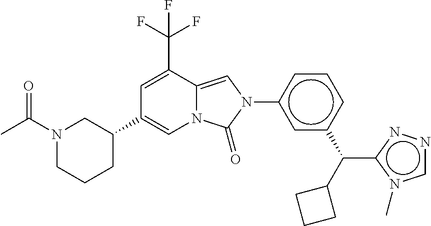 |
| 398 | 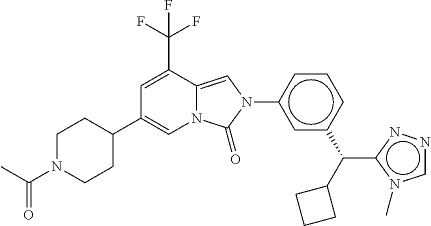 |
| 399 | 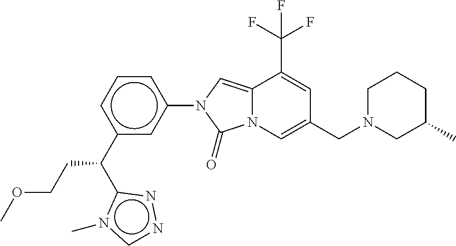 |
| 400 | 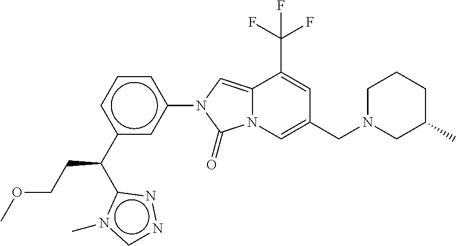 |
| 401_P1 | 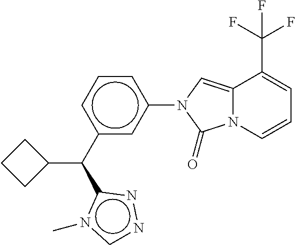 |
| 401_P2 | 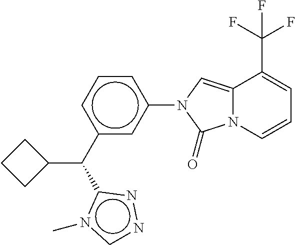 |
| 402 | 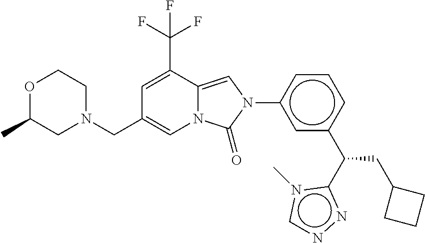 |
| 403_P1 | 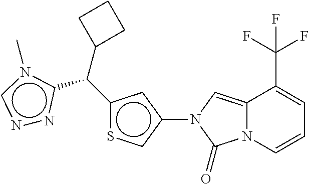 |
| 403_P2 | 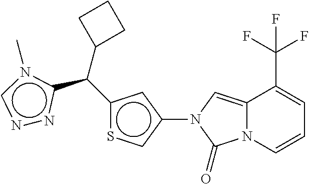 |
| 404 | 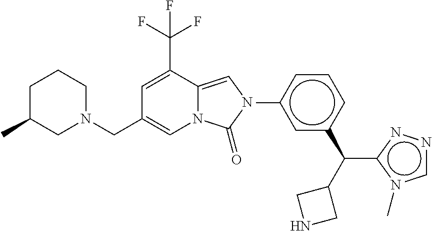 |
| 403 | 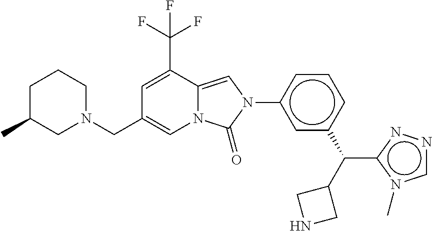 |
| 404 | 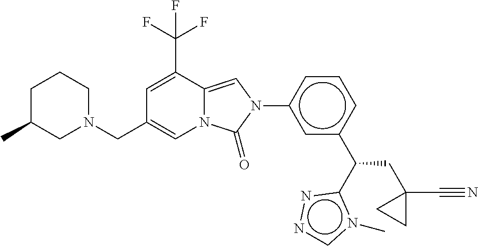 |
| 405 | 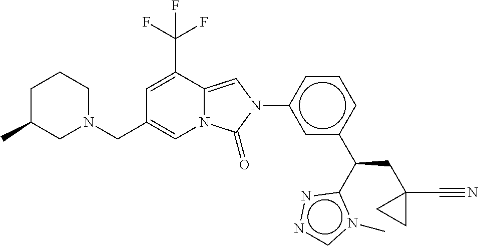 |
| 406 | 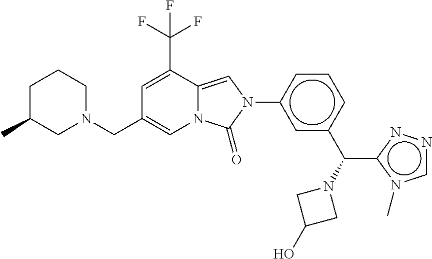 |
| 407 | 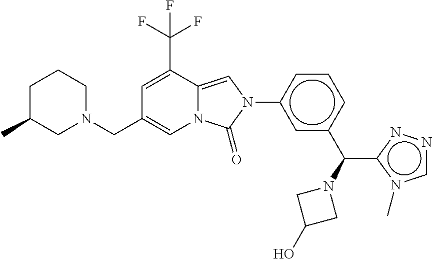 |
| 408 | 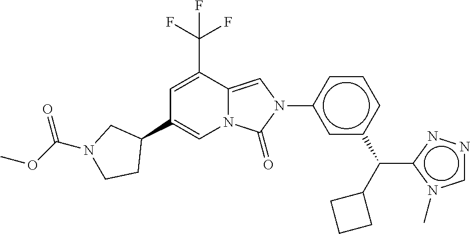 |
| 409 | 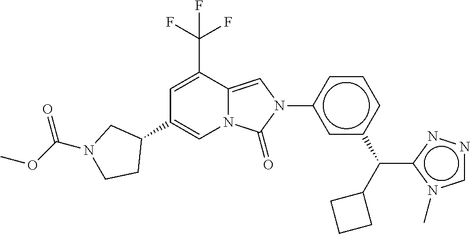 |
| 410 | 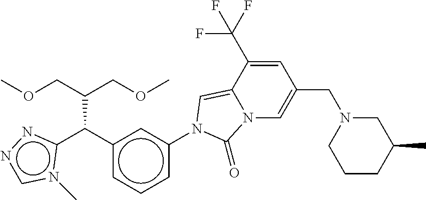 |
| 411 | 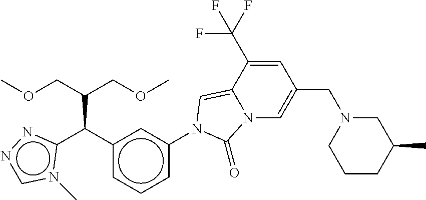 |
| 412 | 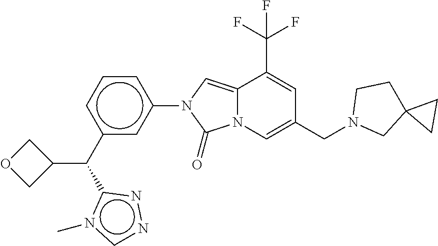 |
| 413 | 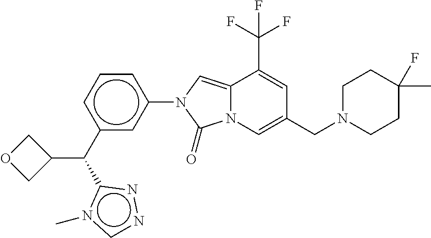 |
| 414 | 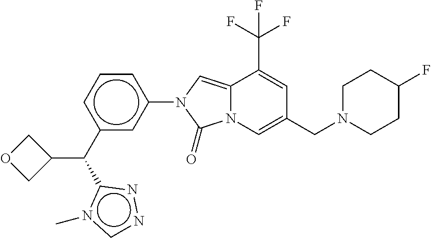 |
| 415 | 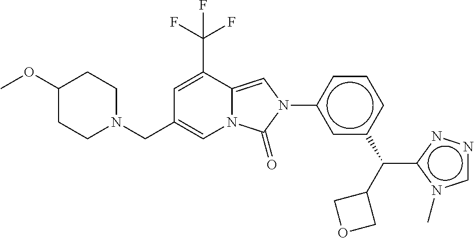 |
| 416 | 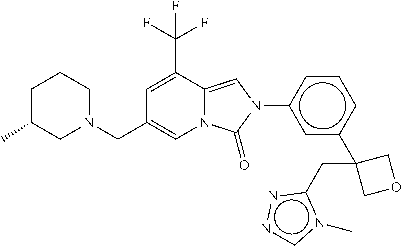 |
| 417 | 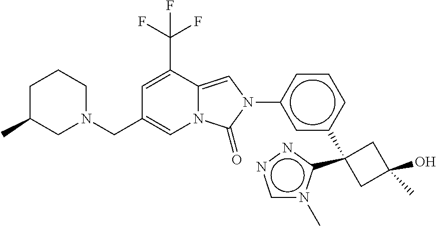 |
| 418 | 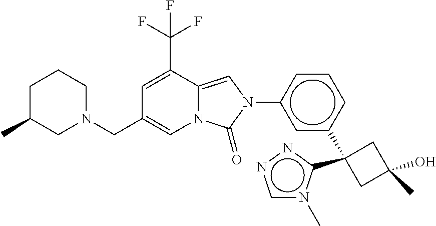 |
| 419 | 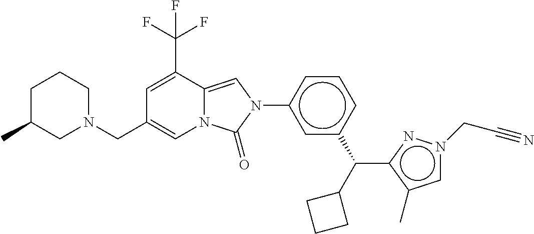 |
| 420 | 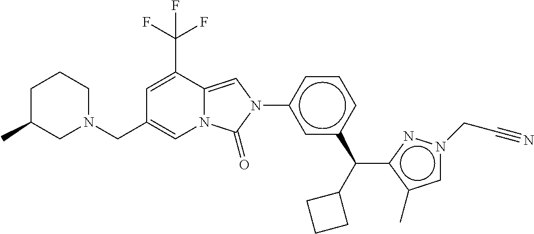 |
| 421 | 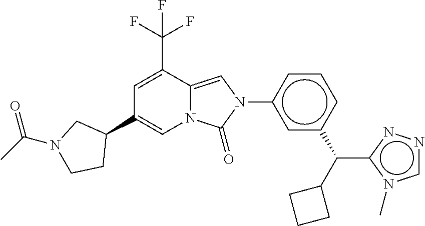 |
| 422 | 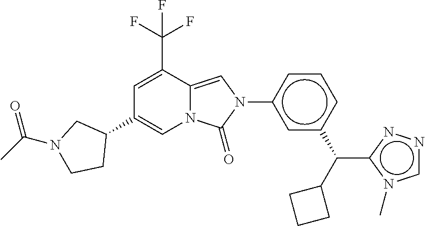 |
| 423 | 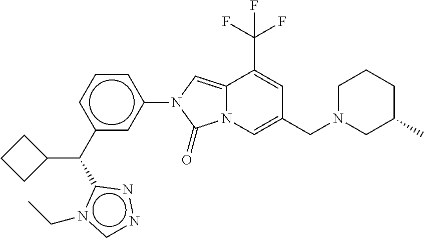 |
| 424 | 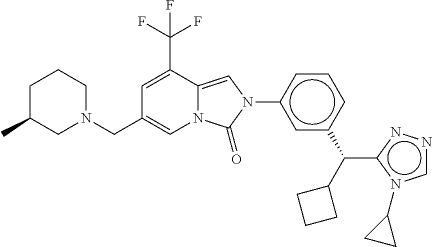 |
| 425 | 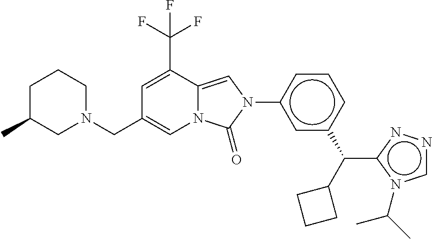 |
| 426 | 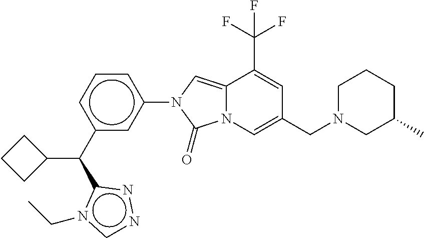 |
| 427 | 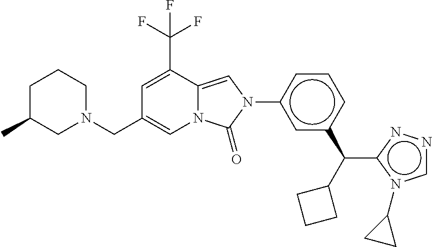 |
| 428 | 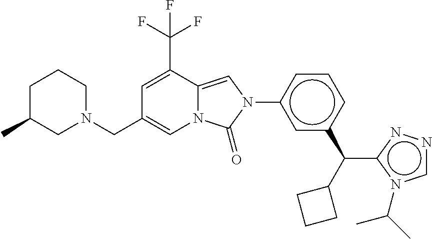 |
| 429 | 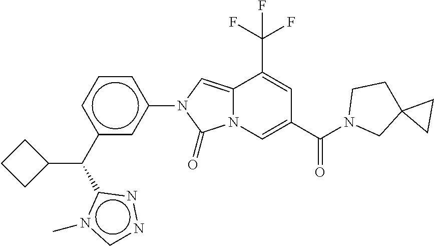 |
| 430 | 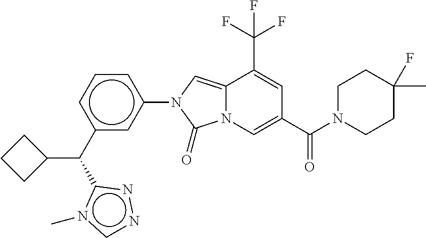 |
| 431 | 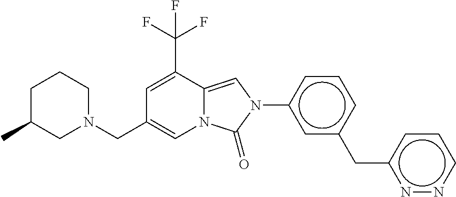 |
| 432 | 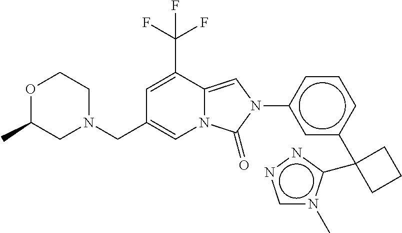 |
| 433 | 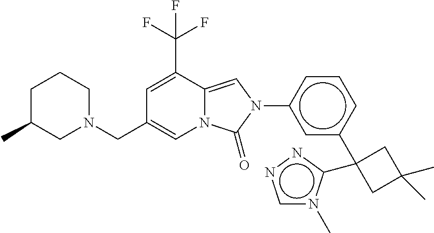 |
| 434_P1 | 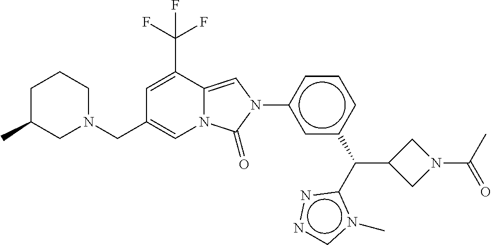 |
| 434_P2 | 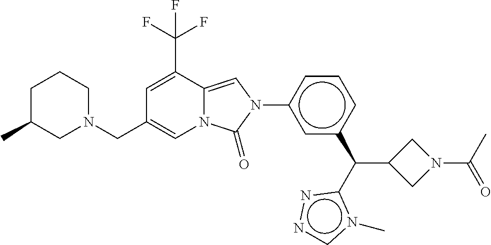 |
| 435 | 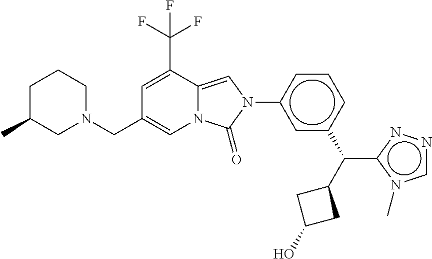 |
| 436 | 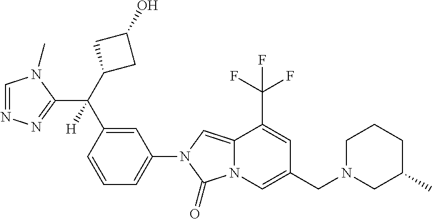 [SMILES: C[C@H]1CCCN(Cc2cc(C(F)(F)F)c3cn(-c4cccc([C@@H](c5nncn5C)[C@H]5C[C@@H](O)C5)c4)c(=O)n3c2)C1] |
| 437 | 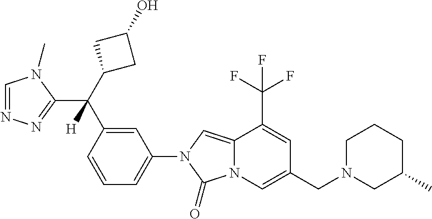 [SMILES: C[C@H]1CCCN(Cc2cc(C(F)(F)F)c3cn(-c4cccc([C@H](c5nncn5C)[C@H]5C[C@@H](O)C5)c4)c(=O)n3c2)C1] |
| 438 | 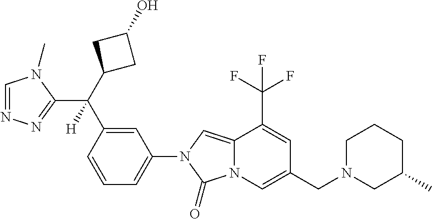 [SMILES: C[C@H]1CCCN(Cc2cc(C(F)(F)F)c3cn(-c4cccc([C@@H](c5nncn5C)[C@H]5C[C@H](O)C5)c4)c(=O)n3c2)C1] |
| 439 | 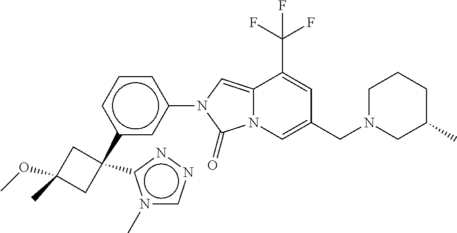 |
| 440 | 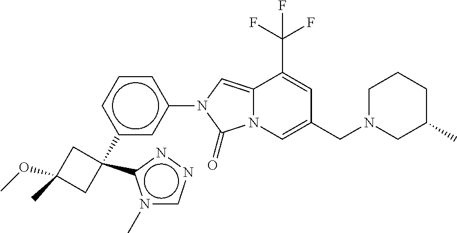 |
| 441 | 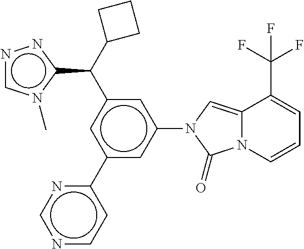 |
| 442 | 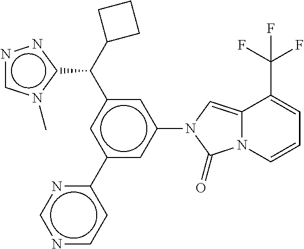 |
| 443 | 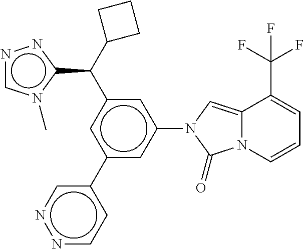 |
| 444 | 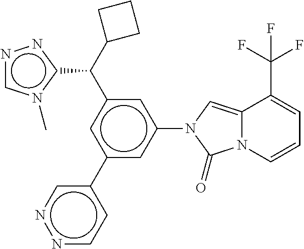 |
| 445 | 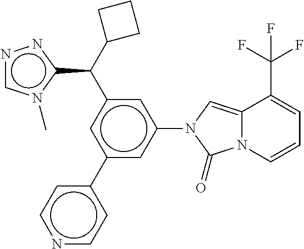 |
| 446 | 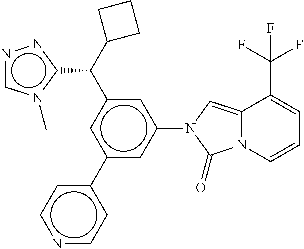 |
| 447 | 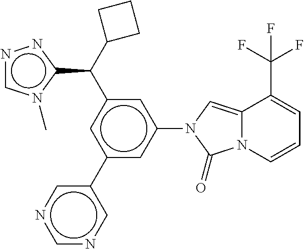 |
| 448 | 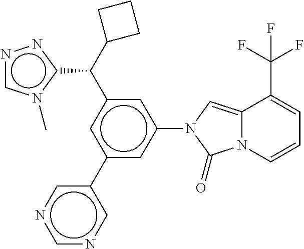 |
| 449 | 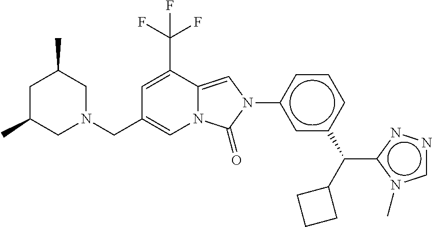 |
| 450 | 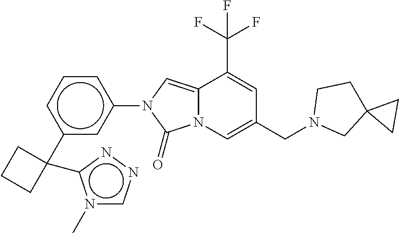 |
| 451 | 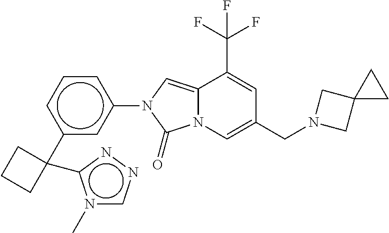 |
| 452 | 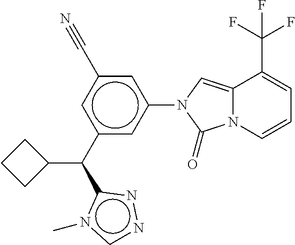 |
| 453 | 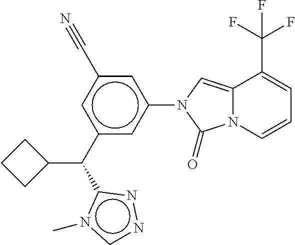 |
| 454 | 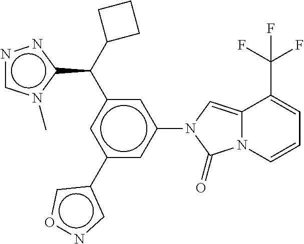 |
| 455 | 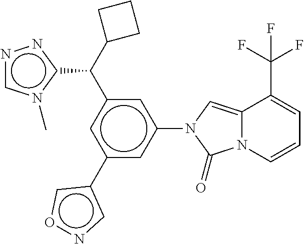 |
| 456 | 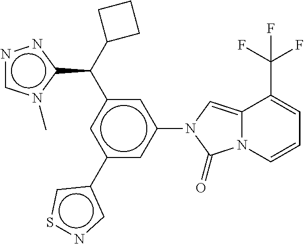 |
| 457 | 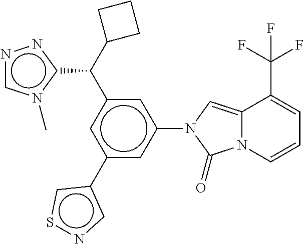 |
| 458 | 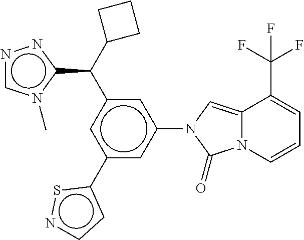 |
| 459 | 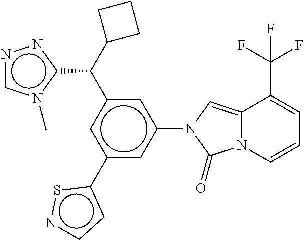 |
| 460 | 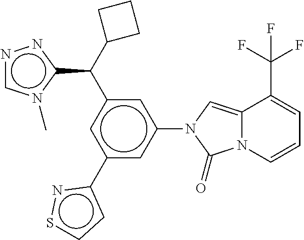 |
| 461 | 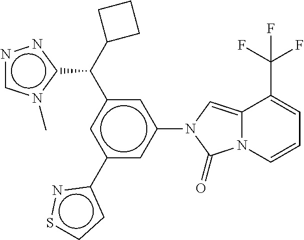 |
| 462 | 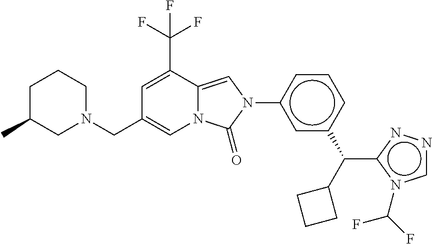 |
| 463 | 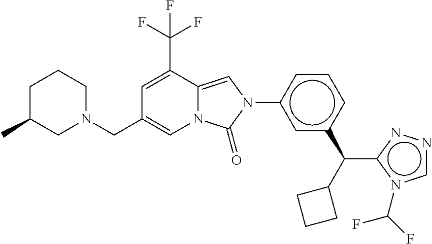 |
| 464 | 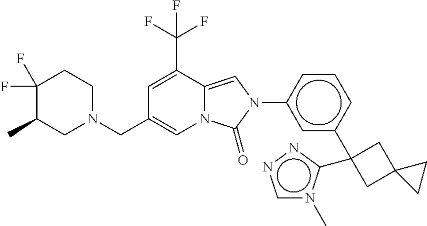 |
| 465 | 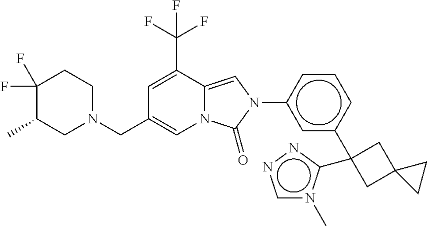 |
| 466 | 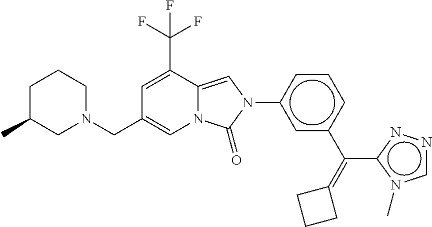 |
| 467 | 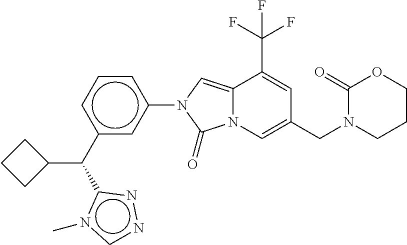 |
| 468 | 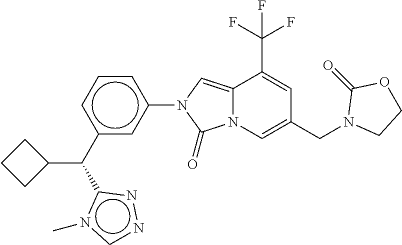 |
| 469 | 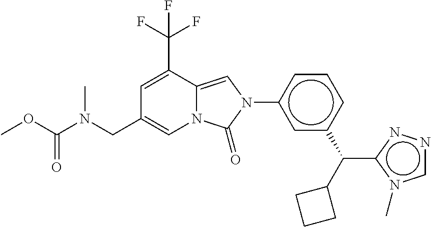 |
| 470_P1 | 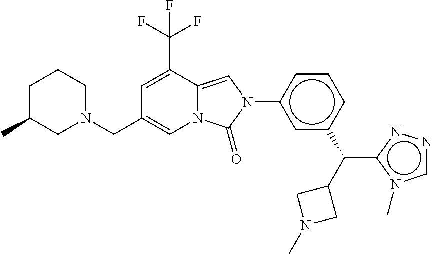 |
| 470_P2 | 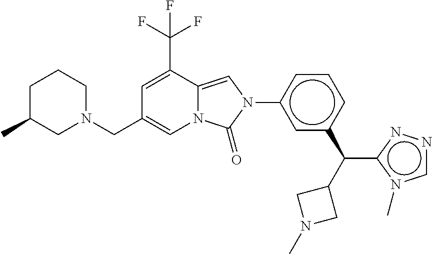 |
| 471 | 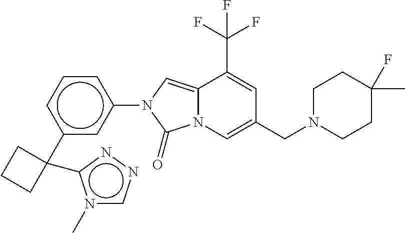 |
| 472_P1 | 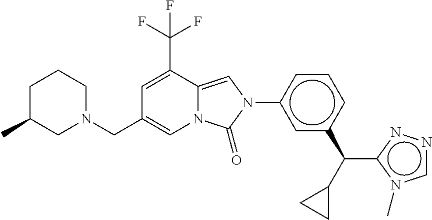 |
| 472_P2 | 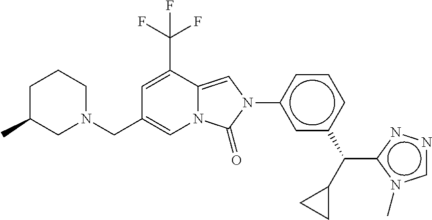 |
| 473 | 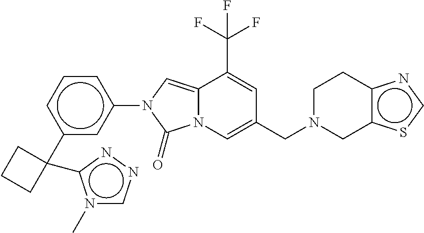 |
| 474 | 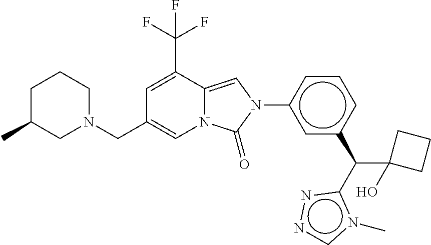 |
| 475 | 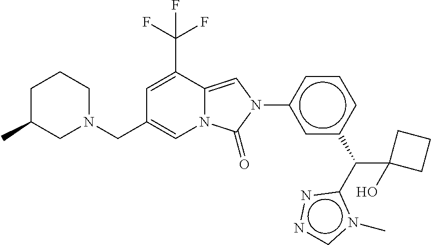 |
| 476 | 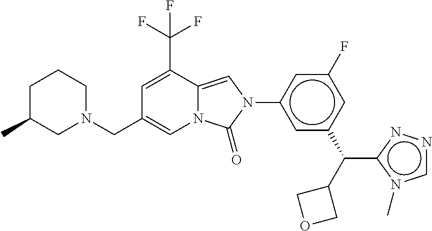 |
| 477 | 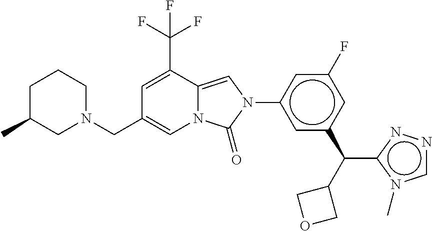 |
| 478 | 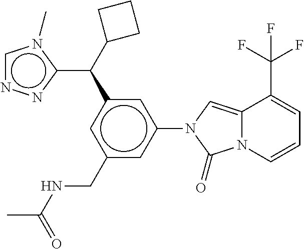 |
| 479 | 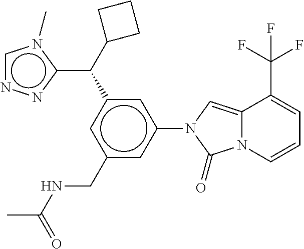 |
| 480 | 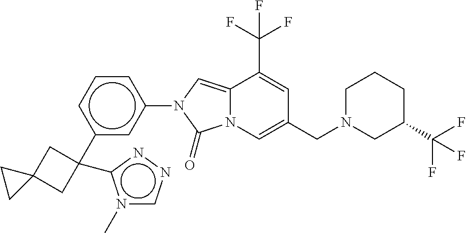 |
| 481 | 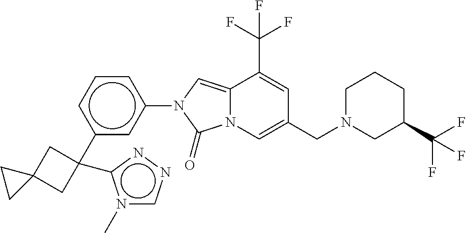 |
| 482 | 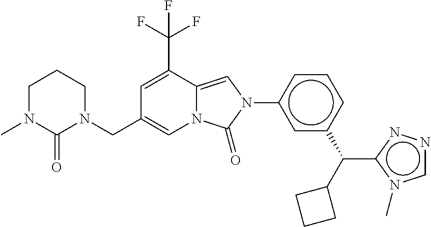 |
| 483 | 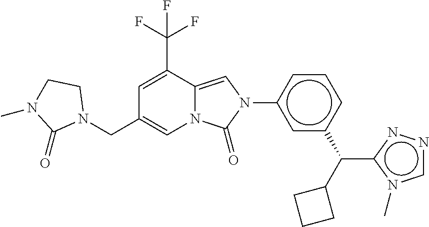 |
| 484 | 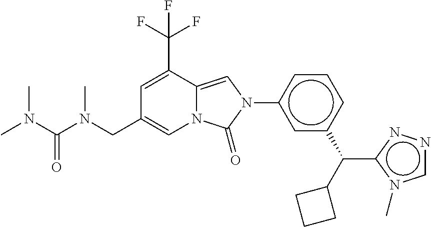 |
| 485 | 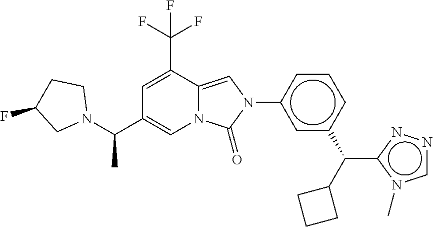 |
| 486 | 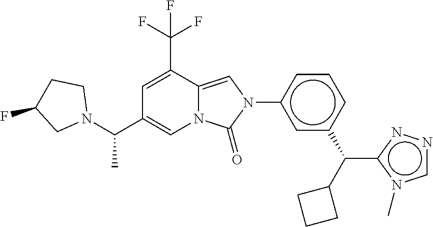 |
| 487 | 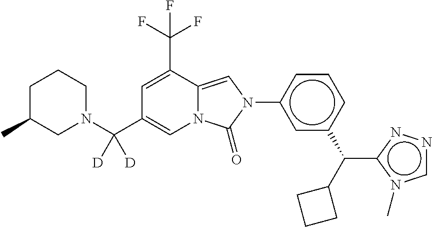 |
| 488 | 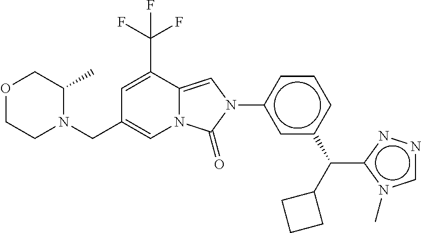 |
| 489 | 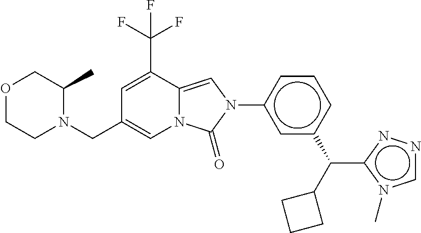 |
| 490 | 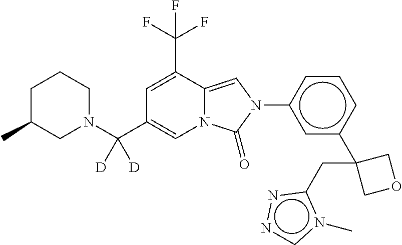 |
| 491 | 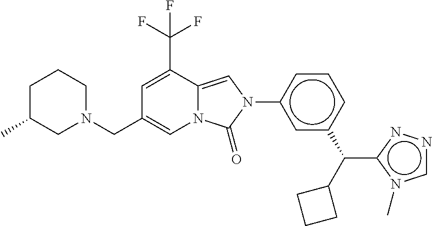 |
| 492 | 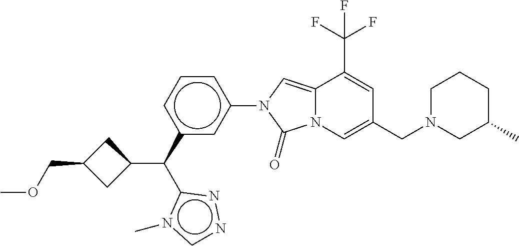 |
| 493 | 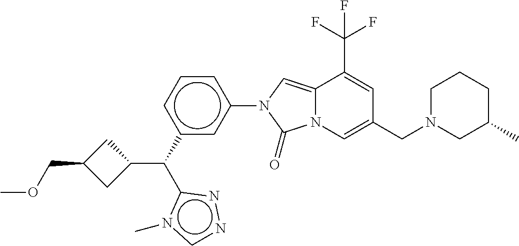 |
| 494 | 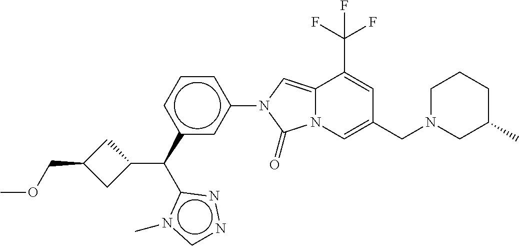 |
| 495 | 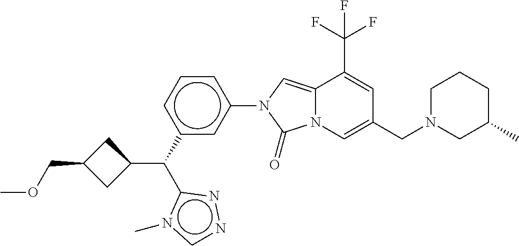 |
| 496 | 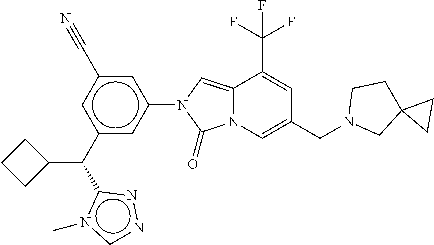 |
| 497 | 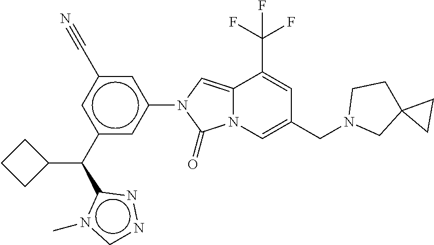 |
| 498 | 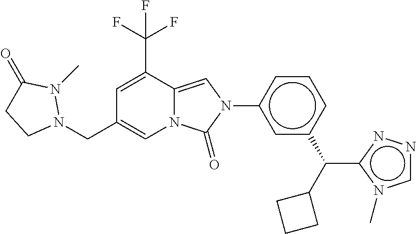 |
| 499 | 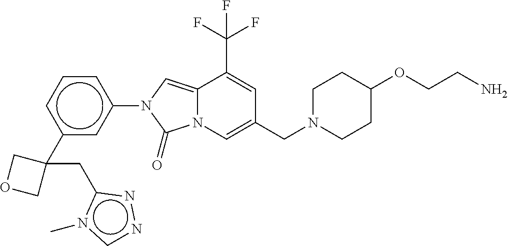 |
| 500 | 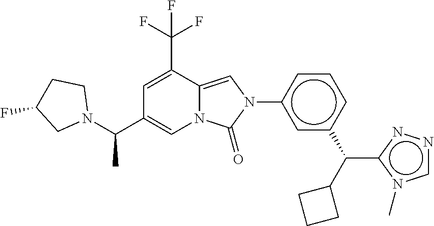 |
| 501 | 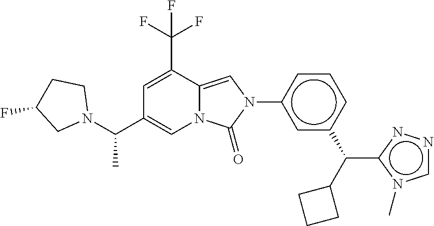 |
| 502 | 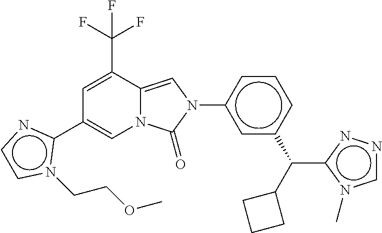 |
| 503 | 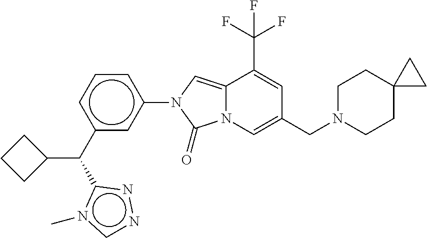 |
| 504 | 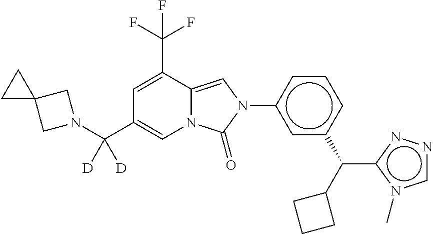 |
| 505 | 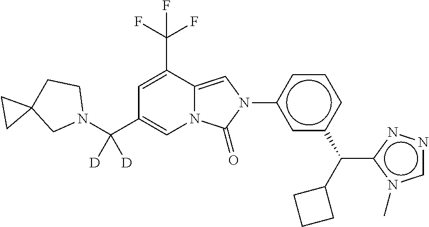 |
| 506_P1 | 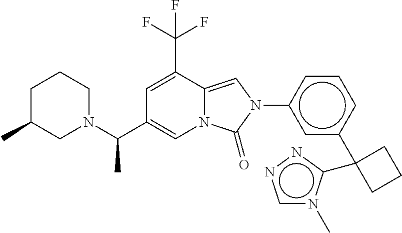 |
| 506_P2 | 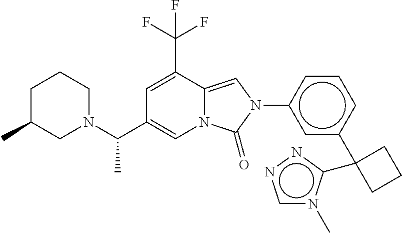 |
| 507 | 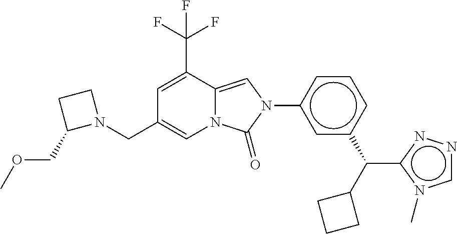 |
| 508 | 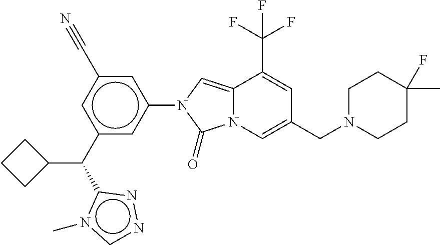 |
| 509 | 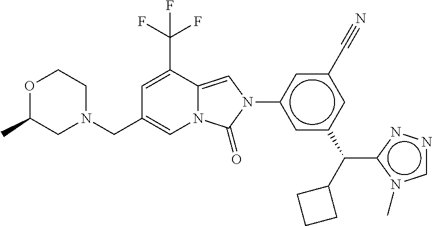 |
| 510 | 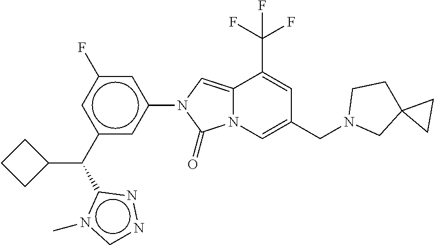 |
| 511 | 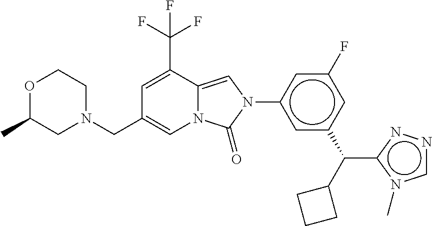 |
| 512 | 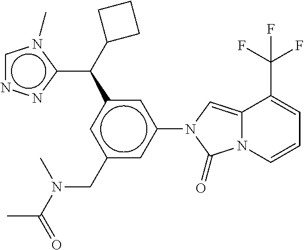 |
| 513 | 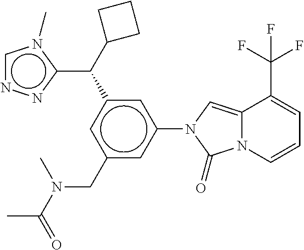 |
| 514 | 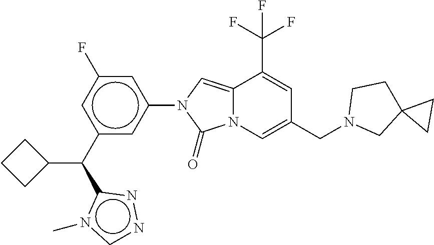 |
| 515 | 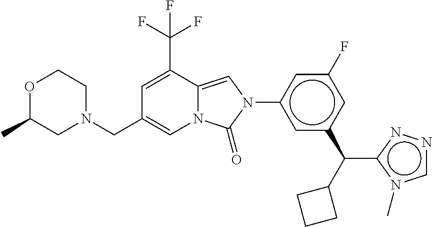 |
| 516 | 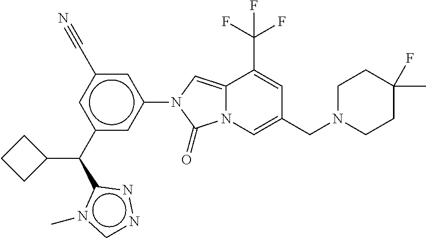 |
| 517 | 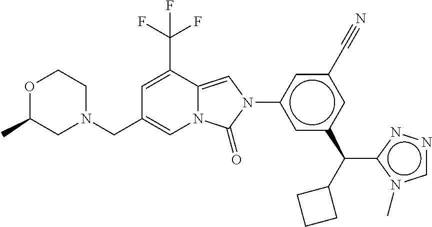 |
| 518 | 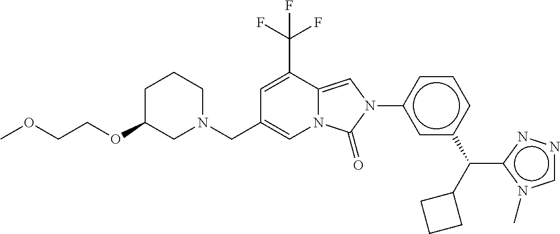 |
| 519 | 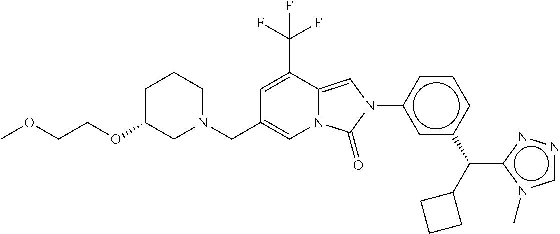 |
| 520 | 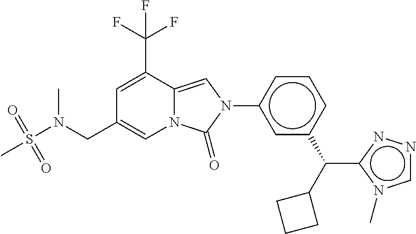 |
| 521 | 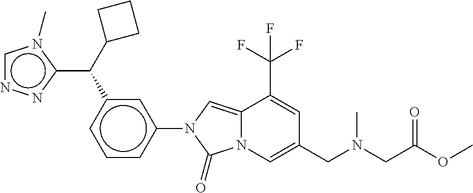 |
| 522 | 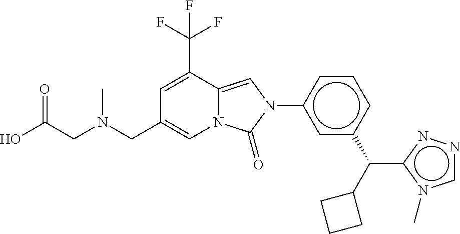 |
| 523 | 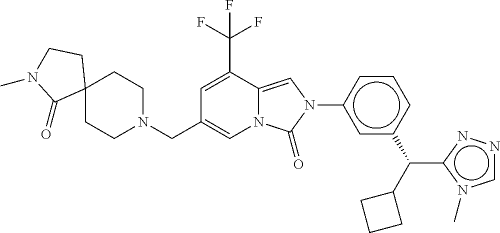 |
| 524 | 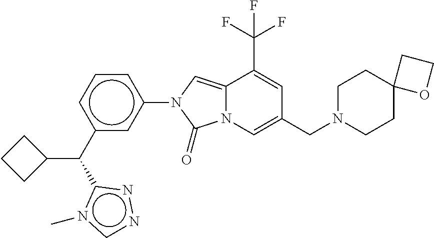 |
| 525 | 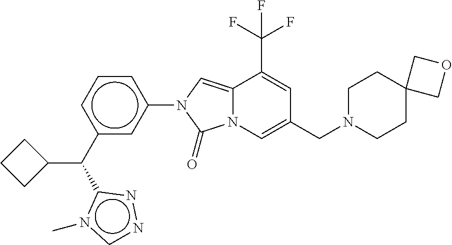 |
| 526 | 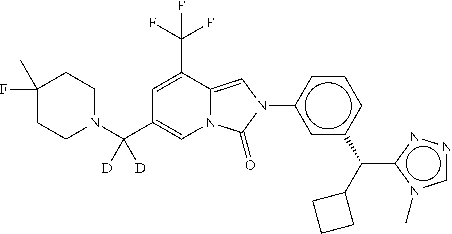 |
| 527 | 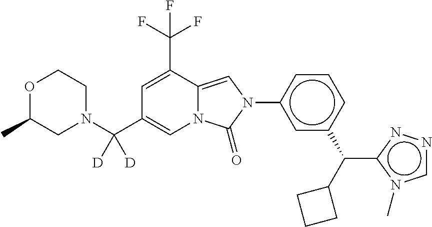 |
| 528 | 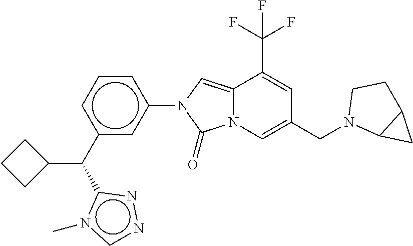 |
| 529 | 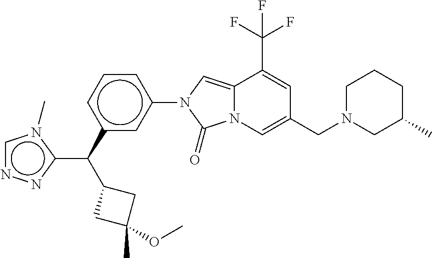 |
| 530 | 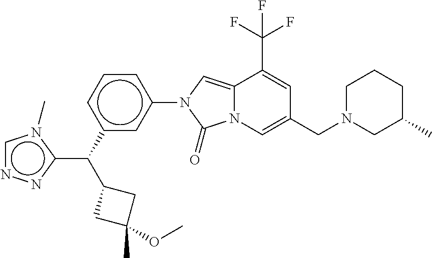 |
| 531 | 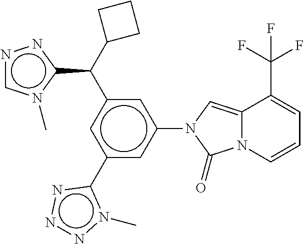 |
| 532 | 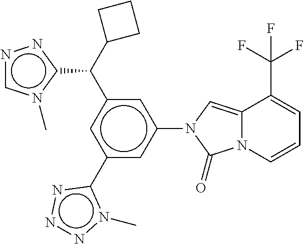 |
| 533 | 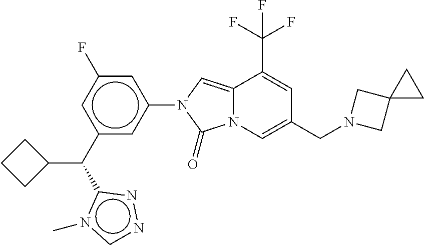 |
| 534 | 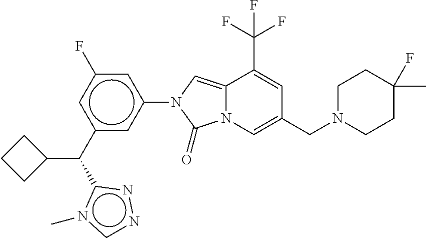 |
| 535 | 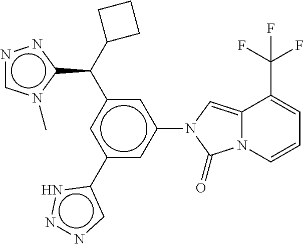 |
| 536 | 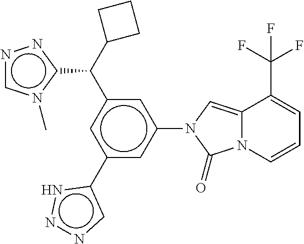 |
| 537 | 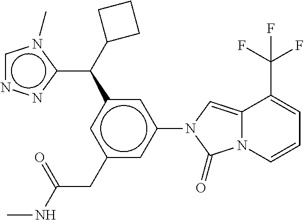 |
| 538 | 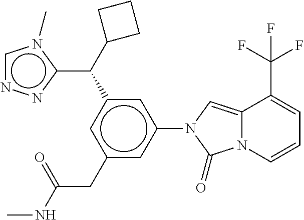 |
| 539 | 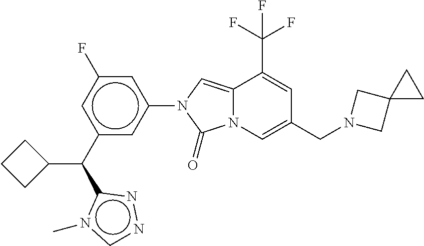 |
| 540 | 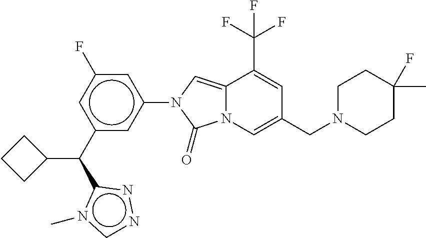 |
| 541 | 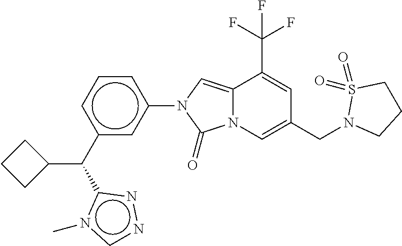 |
| 542 | 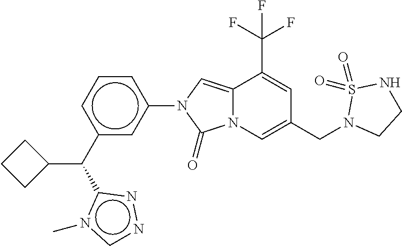 |
| 543 | 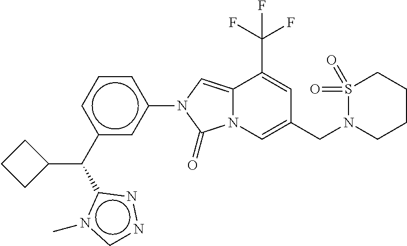 |
| 544 | 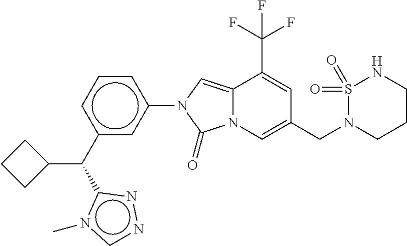 |
| 545 | 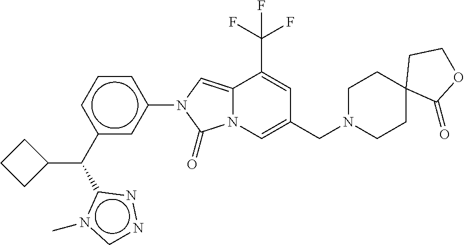 |
| 546 | 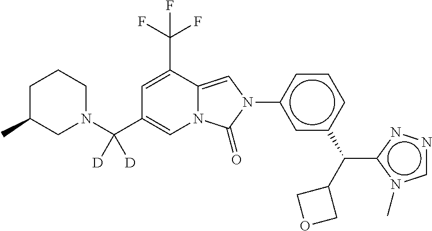 |
| 547 | 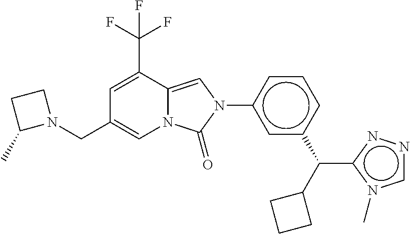 |
| 548 | 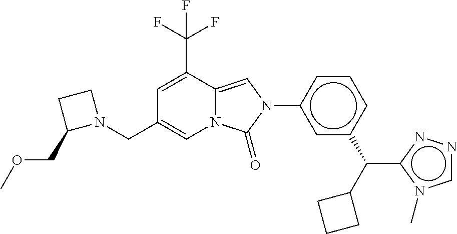 |
| 549 | 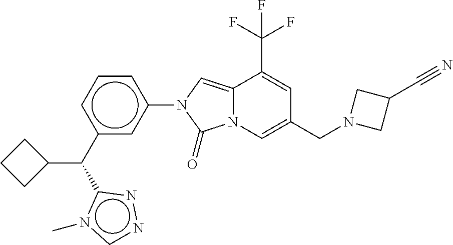 |
| 550_P1 | 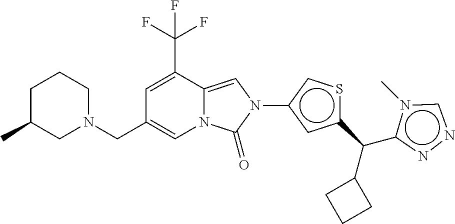 |
| 550_P2 | 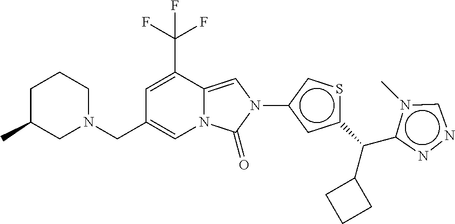 |
| 551 | 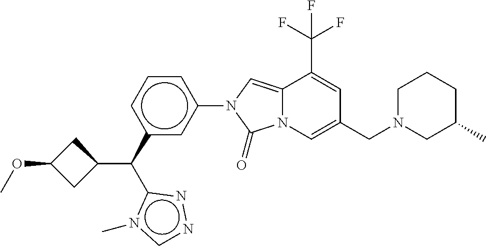 |
| 552 | 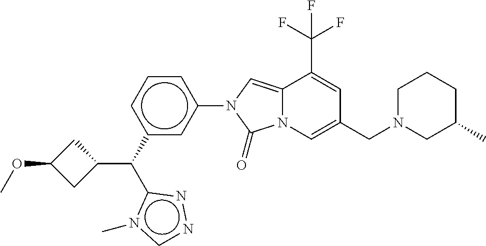 |
| 553 | 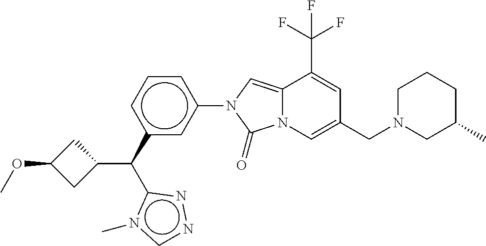 |
| 554 | 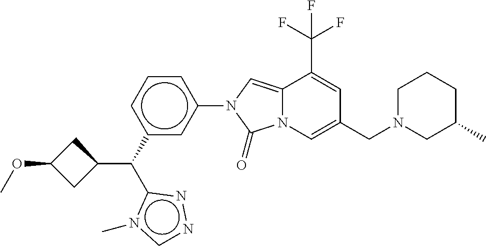 |
| 555 | 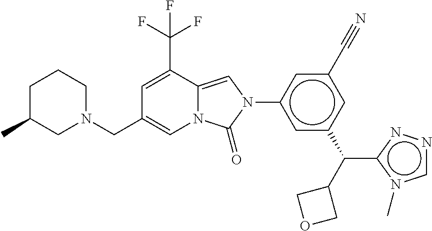 |
| 556 | 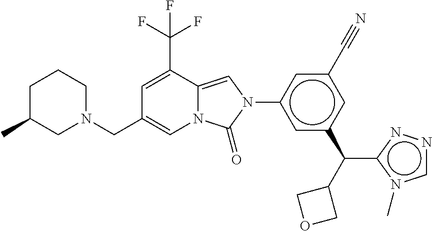 |
| 557 | 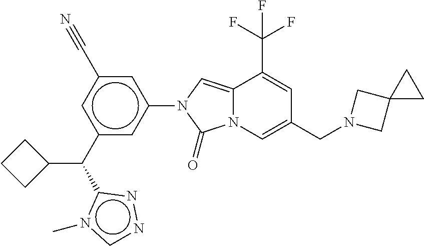 |
| 558 | 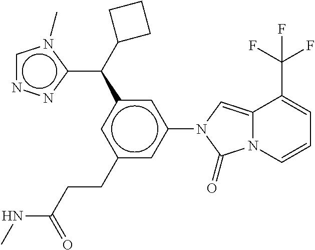 |
| 559 | 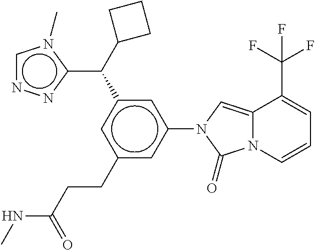 |
| 560 | 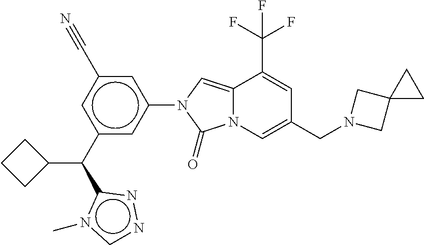 |
| 561 | 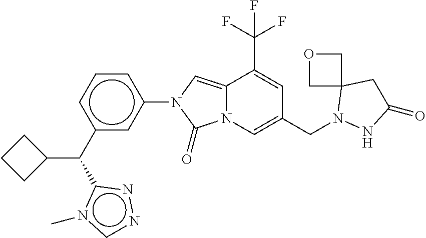 |
| 562 | 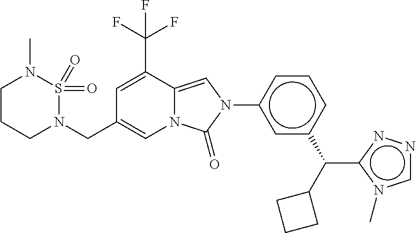 |
| 563 | 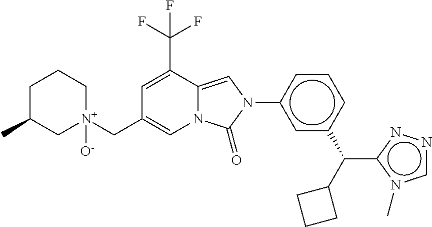 |
| 564 | 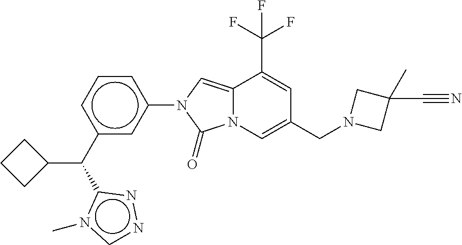 |
| 565_P1 | 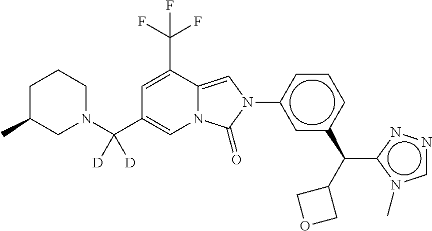 |
| 565_P2 | 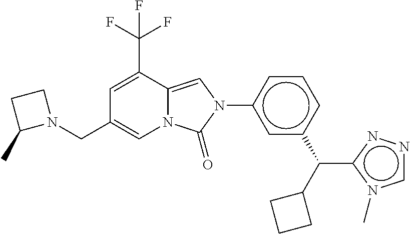 |
| 566 | 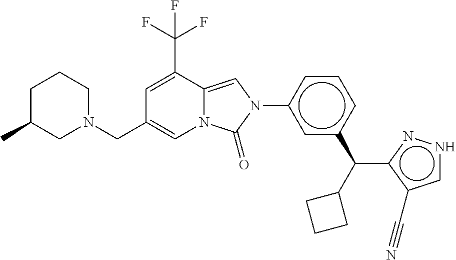 |
| 567 | 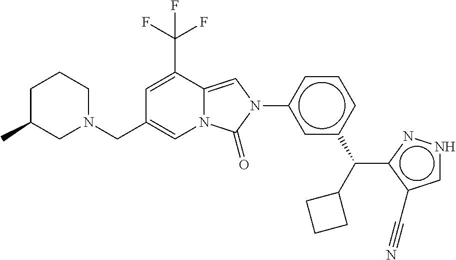 |
| 568 | 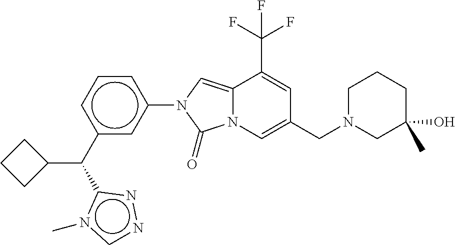 |
| 569 | 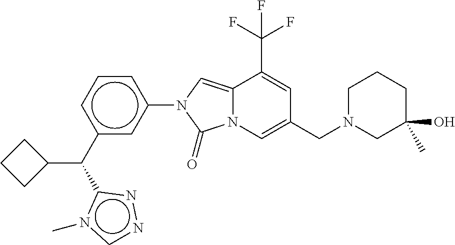 |
| 570 | 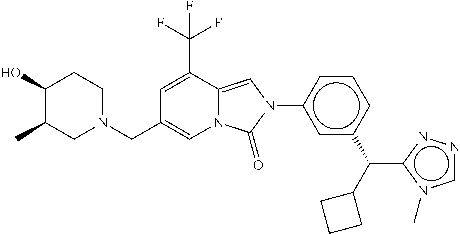 |
| 571 | 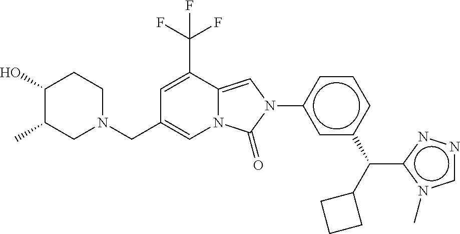 |
| 572 | 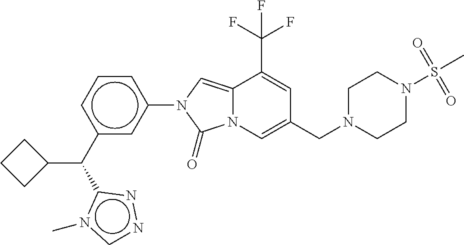 |
| 573_P1 | 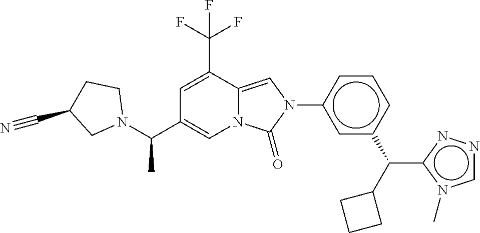 |
| 573_P2 | 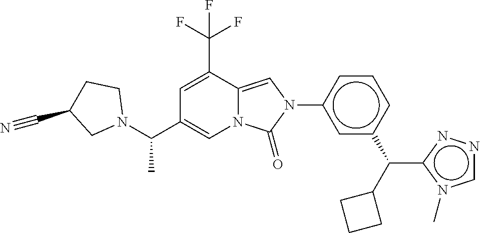 |
| 574 | 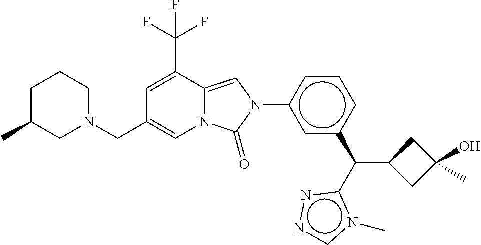 |
| 575 | 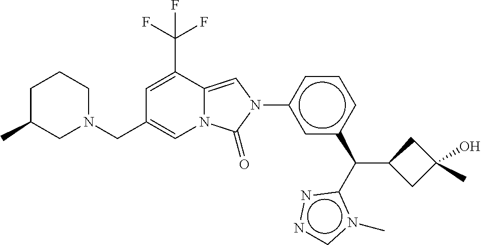 |
| 576 | 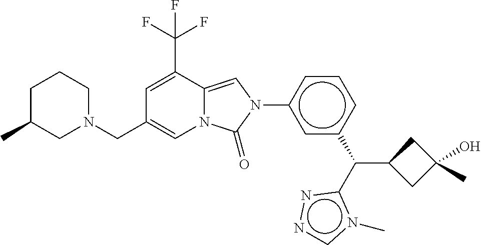 |
| 577 | 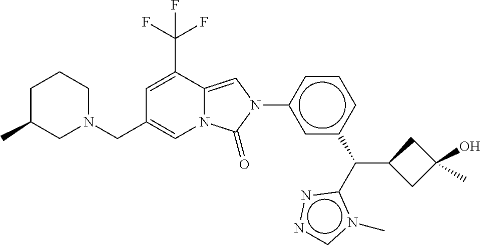 |
| 578 | 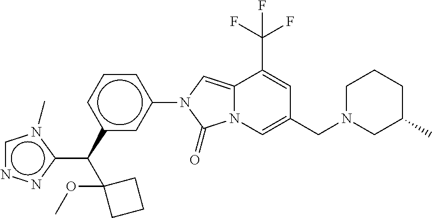 |
| 579 | 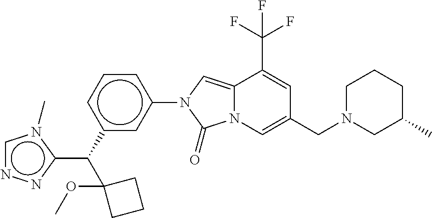 |
| 580 | 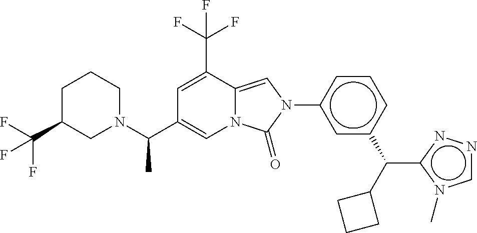 |
| 581 | 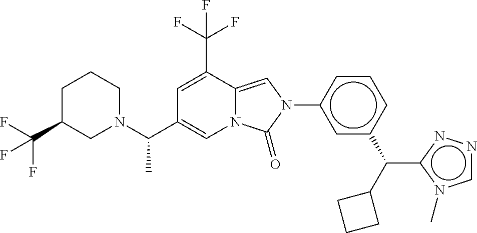 |
| 582 | 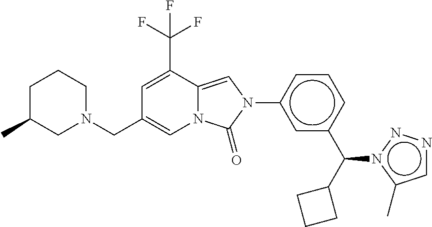 |
| 583 | 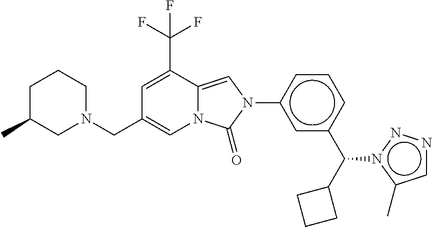 |
| 584 | 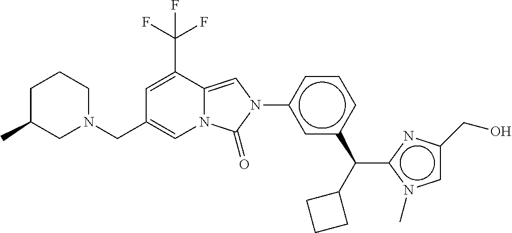 |
| 585 | 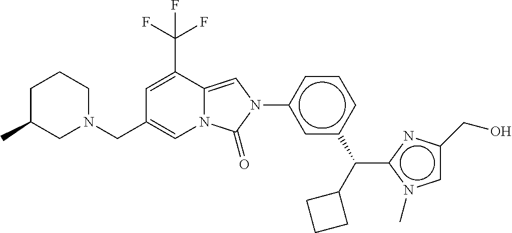 |
| 586 | 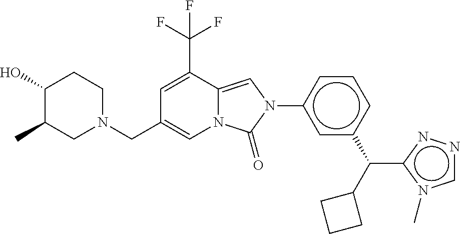 |
| 587 | 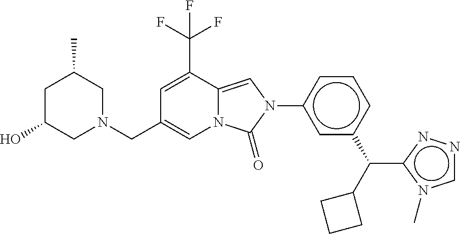 |
| 588 | 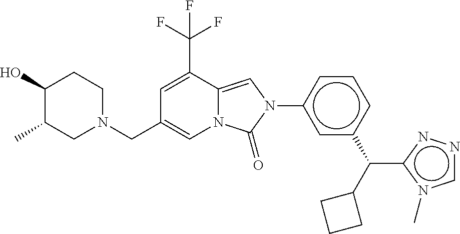 |
| 589 | 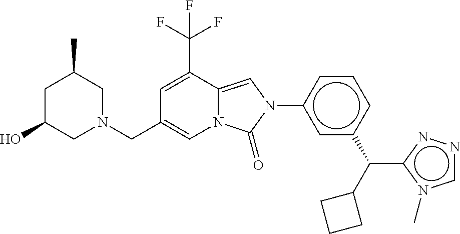 |
| 590 | 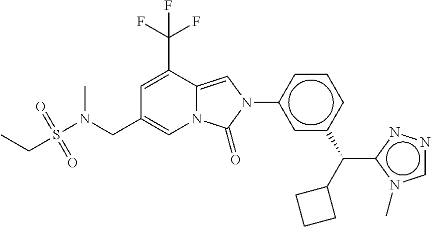 |
| 591 | 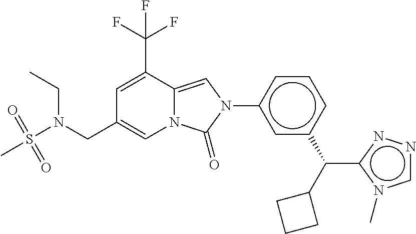 |
| 592 | 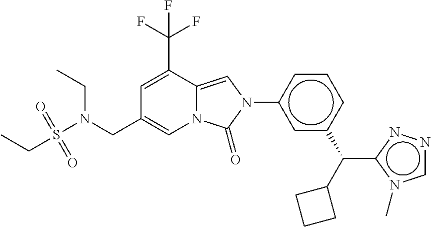 |
| 593 | 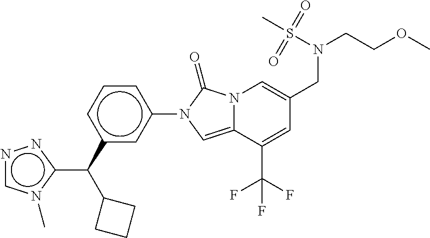 |
| 594 | 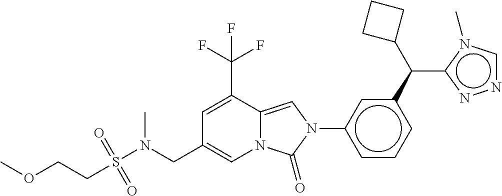 |
| 595 | 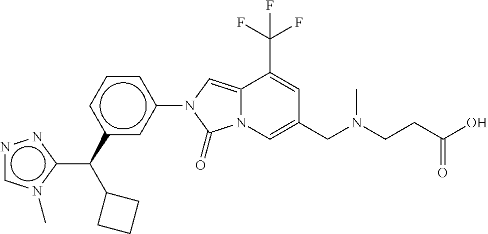 |
| 596 | 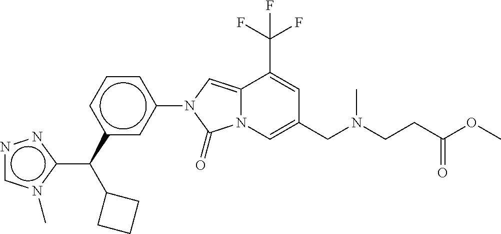 |
| 597_P1 | 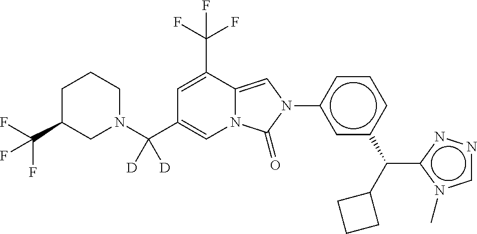 |
| 597_P2 | 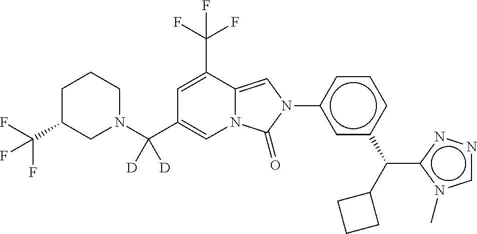 |
| 598 | 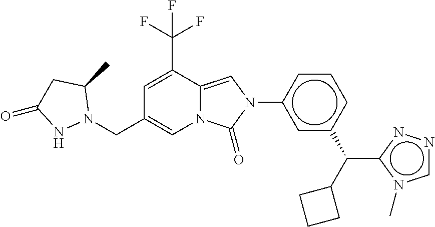 |
| 599 | 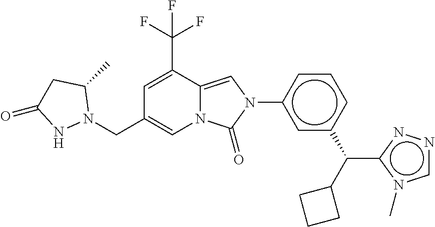 |
| 600 | 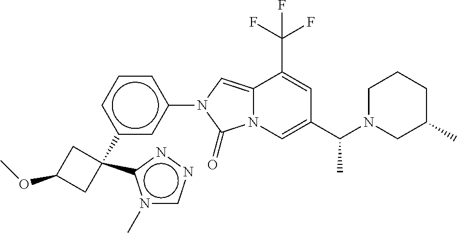 |
| 601 | 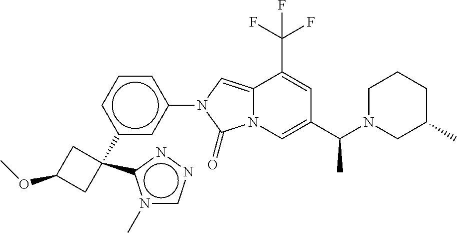 |
| 602 | 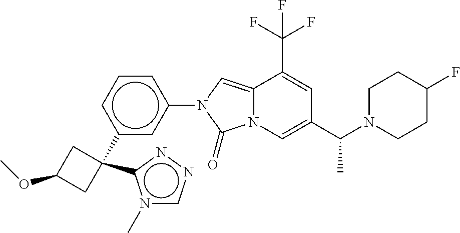 |
| 603 | 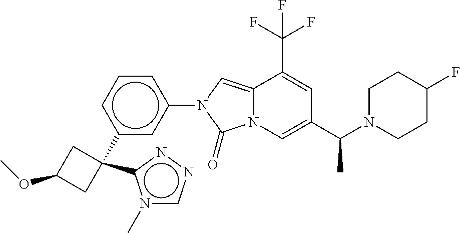 |
| 604 | 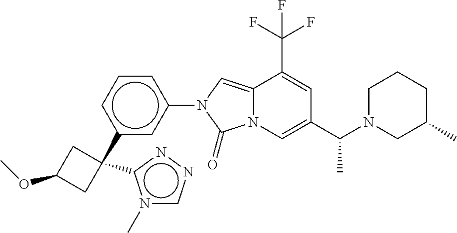 |
| 605 | 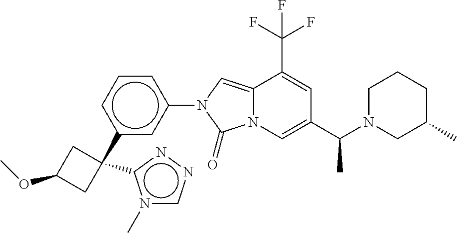 |
| 606 | 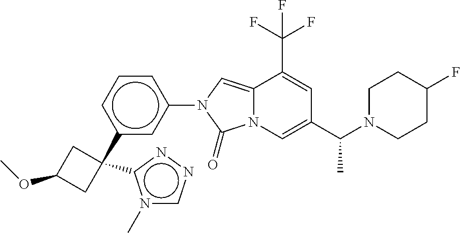 |
| 607 | 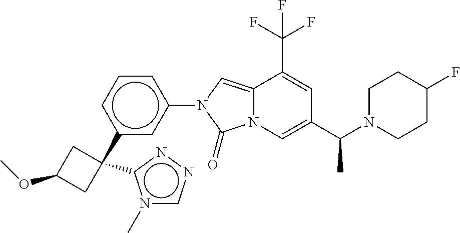 |
| 608 | 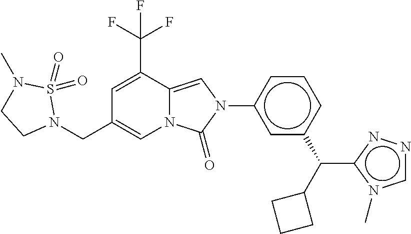 |
| 609 | 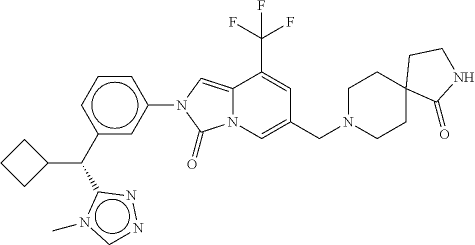 |
| 610_P1 | 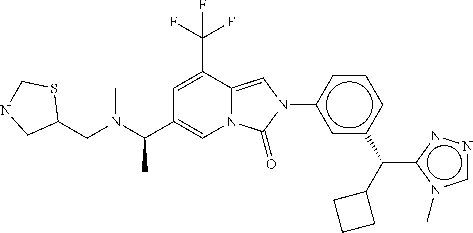 |
| 610_P2 | 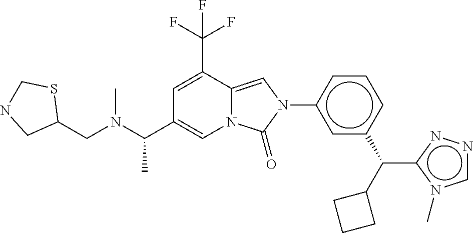 |
| 611 | 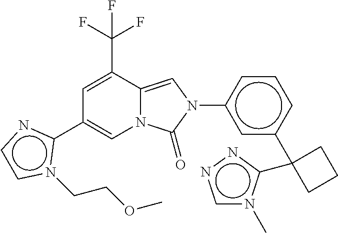 |
| 612_P1 | 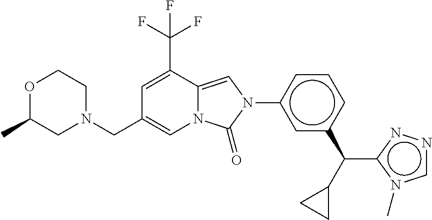 |
| 612_P2 | 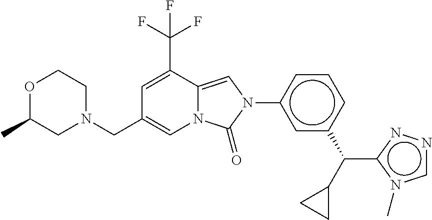 |
| 613 | 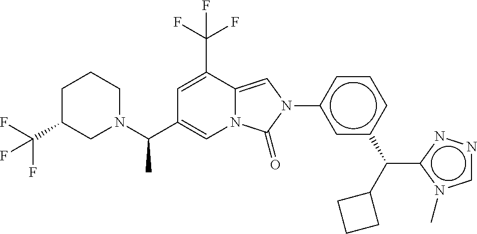 |
| 614 | 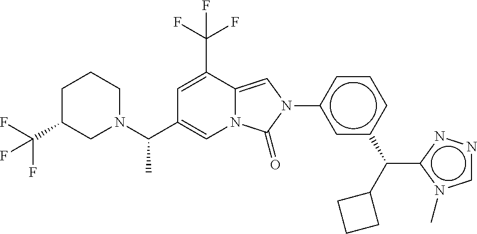 |
| 615 | 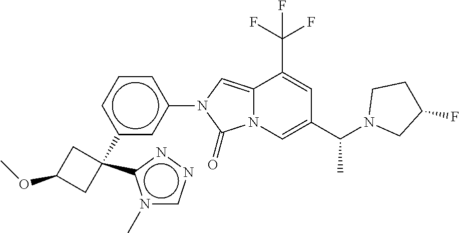 |
| 616 | 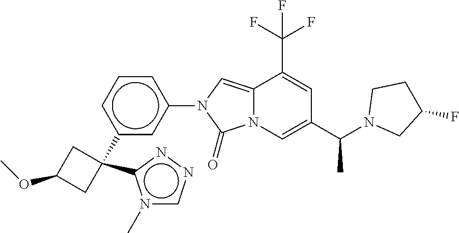 |
| 617 | 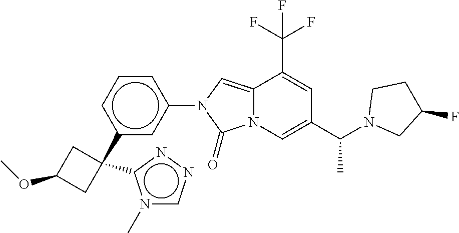 |
| 618 | 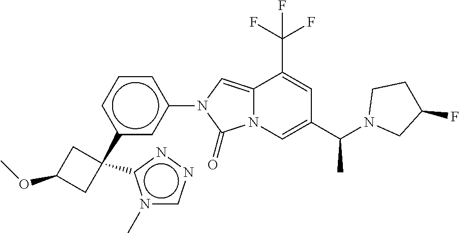 |
| 619 | 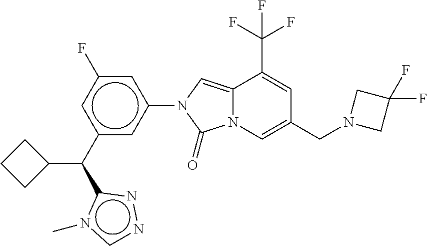 |
| 620 | 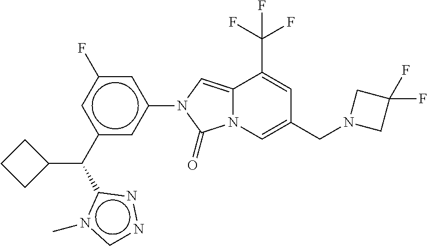 |
| 621 | 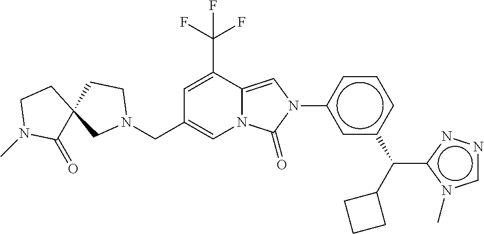 |
| 622 | 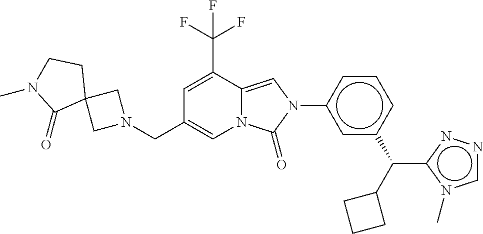 |
| 623 | 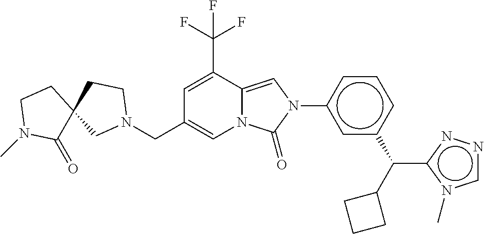 |
| 624 | 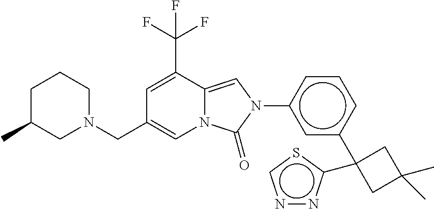 |
| 625 | 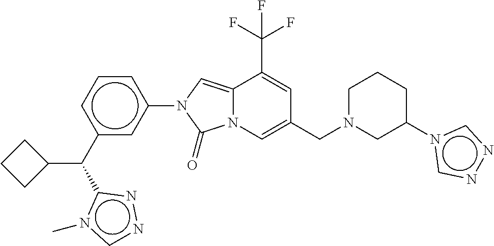 |
| 626 | 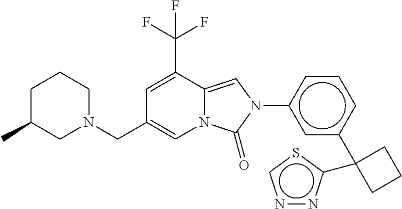 |
| 627_P1 | 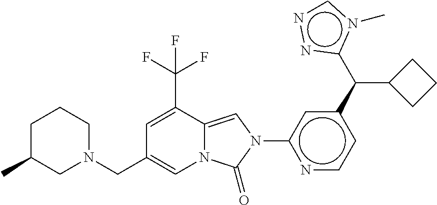 |
| 627_P2 | 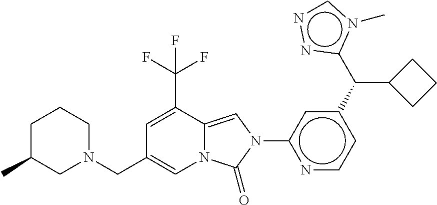 |
| 628_P1 | 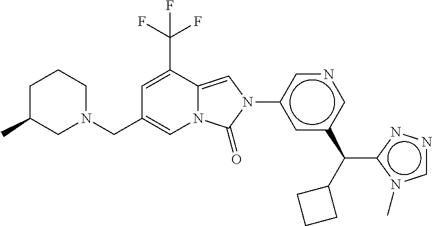 |
| 628_P2 | 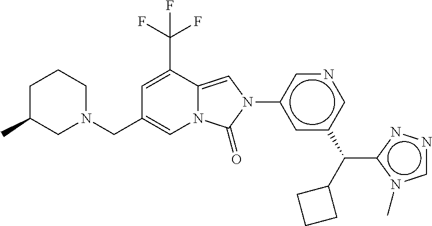 |

or a pharmaceutically acceptable salt thereof

A person of skill in the art will understand the present disclosure includes compounds with the stereochemistry which are the opposite of how they have been drawn. Additionally, the present disclosure contemplates tautomers of the compounds as drawn herein.

The present disclosure includes the racemate of any compound disclosed herein.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; NO₂; —CN; N₃; —(CH₂)₀₋₄N(R^{∘})₂; —(CH₂)₀₋₄N(R^{∘})C(O)R^{∘}; N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; N(R^{∘})N(R^{∘})C(O)R^{∘}; N(R^{∘})N(R^{∘})C(O)NR^{∘}₂; N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; C(S)NR^{∘}₂; —C(S)SR^{∘}; SC(S)SR^{∘}, —(CH₂)₀₋₄OC(O)NR^{∘}₂; —C(O)N(OR^{∘})R^{∘}; C(O)C(O)R^{∘}; C(O)CH₂C(O)R^{∘}; C(NOR^{∘})R^{∘}; —(CH₂)₀₋₄SSR^{∘}; —(CH₂)₀₋₄S(O)₂R^{∘}; (CH₂)₀₋₄S(O)₂OR^{∘}; —(CH₂)₀₋₄OS(O)₂R^{∘}; S(O)₂NR^{∘}₂; —(CH₂)₀₋₄S(O)R^{∘}; N(R^{∘})S(O)₂NR^{∘}₂; N(R^{∘})S(O)₂R^{∘}; N(OR^{∘})R^{∘}; —C(NH)NR^{∘}₂; P(O)₂R^{∘}; P(O)R^{∘}₂; OP(O)R^{∘}₂; —OP(O)(OR^{∘})₂; SiR^{∘}₃; —(C₁₋₄ straight or branched alkylene)O N(R^{∘})₂; or (C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), (CH₂)₀₋₂OH, —(CH₂)₀₋₂OR^{●}, —(CH₂)₀₋₂CH(OR^{●})₂; —O(haloR^{●}), —CN, N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, NR*, NOR*, —O(C(R*₂))₂₋₃O—, or —S(C(R*₂))₂₋₃S, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, R^{●}, -(haloR^{●}), —OH, —OR^{●}, —O(haloR^{●}), —CN, —C(O)OH, —C(O)OR^{●}, NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, R^{●}, -(haloR^{●}), —OH, —OR^{●}, —O(haloR^{●}), —CN, —C(O)OH, —C(O)OR^{●}, NH₂, NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is hyperplasia or cancer. In some embodiments, a disease or condition associated with cell proliferation is cancer.

In some embodiments, administration of a compound of the present disclosure results in arrest of mitosis.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is a hematologic cancer. In some embodiments, a hematologic cancer is selected from a group consisting of lymphoma, leukemia, and myeloma. In some embodiments, a hematologic cancer is lymphoma. In some embodiments, a hematologic cancer is leukemia. In some embodiments, a hematologic cancer is myeloma.

In some embodiments, cancer is a non-hematologic cancer. In some embodiments, a non-hematologic cancer is a sarcoma or a carcinoma. In some embodiments, a non-hematologic cancer is a sarcoma. In some embodiments, a non-hematologic cancer is carcinoma.

In some embodiments, a subject has one or more of increased T-cell activation, increased T-cell proliferation, decreased T-cell exhaustion, decreased T-cell anergy and decreased T-cell tolerance after administration of compound of the present disclosure. In some embodiments, administration of a compound of the present disclosure to a subject in need there of results in one or more of increased T-cell activation, increased T-cell proliferation, decreased T-cell exhaustion, decreased T-cell anergy and decreased T-cell tolerance.

In some embodiments, a subject has increased NK-cell activation. In some embodiments, increased NK-cell activation comprises increased production of cytokines.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### EXEMPLIFICATION

### Intermediate I-1: Synthesis of Intermediate 1

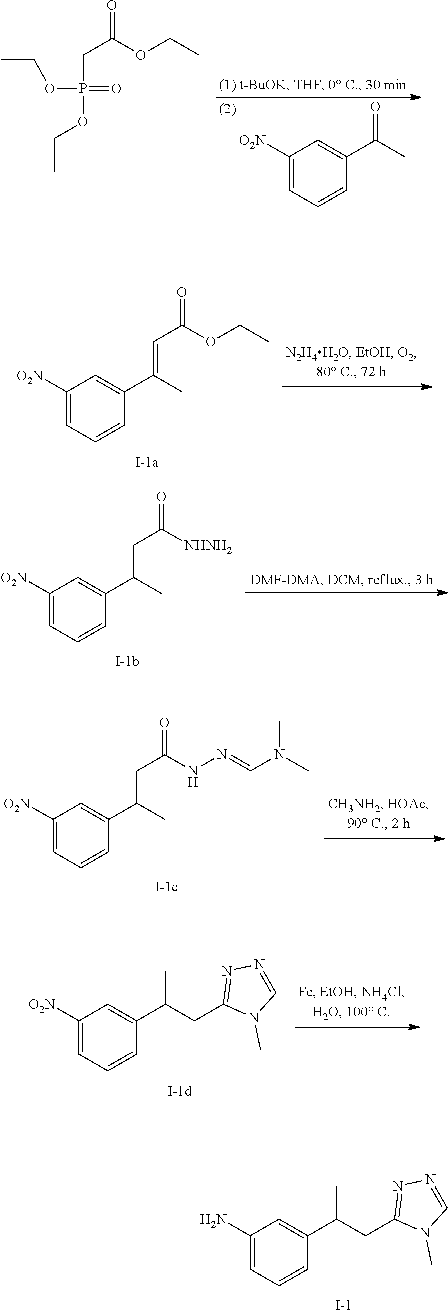

### Synthesis of I-1a

To a stirred mixture of triethyl phosphonoacetate (4.00 g, 17.842 mmol, 1.00 equiv) in THE (50.00 mL) was added t-BuOK (2.00 g, 17.842 mmol, 1.00 equiv) at 0° C. The resulting mixture was stirred for 30 min at 0° C. under argon atmosphere. Then 3-nitroacetophenone (0.97 g, 5.888 mmol, 0.33 equiv) was added, the resulting mixture was stirred overnight at room temperature under nitrogen atmosphere. The resulting mixture was diluted with water (150 mL), extracted with EA (2×100 mL). The combined organic layers were dried over anhydrous Na₂SO₄. After filtration, the filtrate was concentrated under reduced pressure. The residue was purified by silica gel column chromatography, eluted with PE/EtOAc (10:1) to afford I-1a (1.2 g, 28.63%) as an orange oil.

### Synthesis of I-1b

To a stirred mixture of Ia (1.20 g, 5.101 mmol, 1.00 equiv) in EtOH (20.00 mL) was added N₂H₄·H₂O (2.57 g, 51.012 mmol, 10.00 equiv) in 1 portion at room temperature. The resulting mixture was stirred for 72 h at 80° C. under oxygen. The resulting mixture was concentrated under reduced pressure. The residue was dissolved with EA (30 mL), washed with water (2×10 mL), dried over anhydrous Na₂SO₄. After filtration, the filtrate was concentrated under reduced pressure. The residue was purified by silica gel column chromatography, eluted with CH₂Cl₂/MeOH (10:1) to afford I-1b (1.00 g, 82.5%) as an orange oil.

### Synthesis of I-1c

To a stirred mixture of I-1b (500.00 mg, 2.240 mmol, 1.00 equiv) in DCM (10.00 mL) was added DMF-DMA (1.07 g, 8.959 mmol, 4.00 equiv) at room temperature. The resulting mixture was stirred for 3 h at 40° C. under argon atmosphere. The resulting mixture was concentrated under reduced pressure. The resulting mixture was dissolved with EA (50 mL), washed with water (10 mL), dried over anhydrous Na₂SO₄. After filtration, the combined organic layers were concentrated under reduced pressure. The residue was purified by trituration with CH₂Cl₂/MeOH=10/1 (200 mL). The resulting mixture was concentrated under reduced pressure to afford I-1c (500 mg, 80.25%) as an orange oil.

### Synthesis of I-1d

To a stirred mixture of I-1c (500.00 mg, 1.796 mmol, 1.00 equiv) in HOAc (5.00 ml) was added CH₃NH₂ (5.00 ml, 2M in THF) at room temperature. The resulting mixture was stirred for 2 h at 40° C. under argon atmosphere. The resulting mixture was diluted with EA (60 mL), washed with water (10 mL), dried over anhydrous Na₂SO₄. After filtration, the combined organic layers were concentrated under reduced pressure. The residue was purified by trituration with CH₂Cl₂/MeOH=10/1 (200 mL). The resulting mixture was concentrated under reduced pressure to afford I-1d (380 mg, 85.97%) as an orange oil.

### Synthesis of I-1

To a stirred mixture of I-1d (380 mg, 2.030 mmol, 1.00 equiv) in EtOH (5.00 mL) were added NH₄Cl (100.00 mg, 1,869 mmol, 0.92 equiv), H₂O (5.00 mL) and Fe (566.91 mg, 10.151 mmol, 5.00 equiv) at room temperature. The resulting mixture was stirred overnight at 100° C. under nitrogen atmosphere. The reaction was quenched with NaHCO₃ (aq.) at 0° C. The resulting mixture was extracted with EA (3×10 mL), dried over anhydrous Na₂SO₄. After filtration, the filtrate was concentrated under reduced pressure. The crude product (150 mg) was purified by reverse flash chromatography with the following conditions: column, C18; mobile phase, A: water (0.05% NH₃·H₂O), B: CH₃CN, 3% B to 23% B gradient in 20 min; detector, UV 254 nm. This resulted in product. These product and Si-thiol (20 mg) in THE (3 mL) was stirred at room temperature for 30 min. The mixture was filtered and the filtrate was concentrated under reduced pressure. The residue was dried by lyophilization to afford I-1 (86.6 mg, 26.01%) as an orange oil.

LC-MS: (ES, m/z): [M+H]⁺:216

¹H NMR: (400 MHz, DMSO-d6, ppm): δ 1.19-1.21 (d, 3H), 2.86-2.88 (d, 2H), 2.99-3.04 (m, 1H), 3.31-3.38 (m, 3H), 4.95 (s, 1H), 6.36-6.42 (m, 3H), 6.88-6.93 (m, 1H), 8.27 (s, 1H).

### Intermediate I-2: Synthesis of Intermediate 2

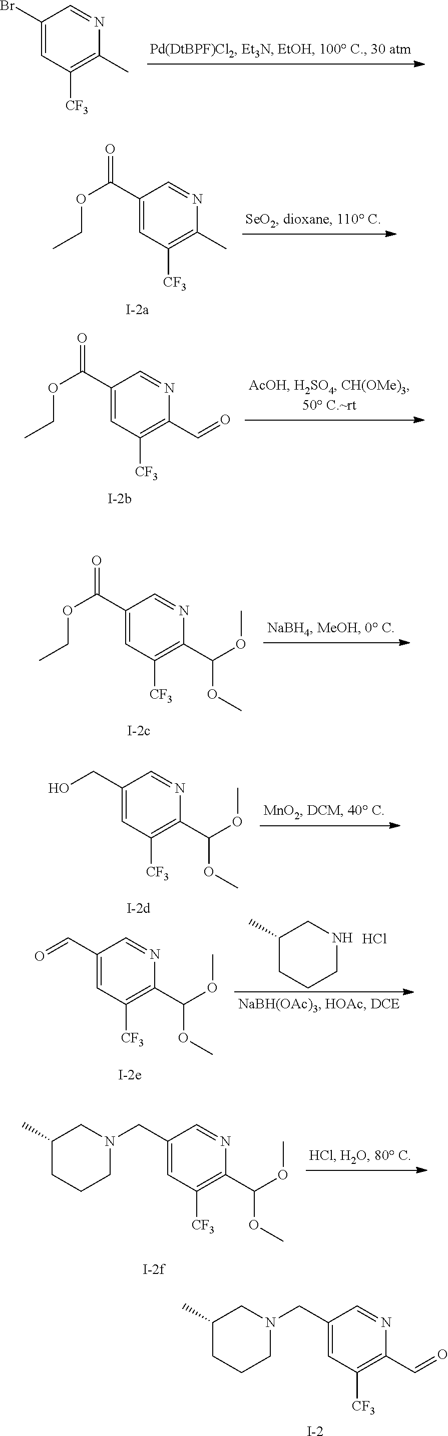

### Synthesis of I-2a

To a solution of 5-bromo-2-methyl-3-(trifluoromethyl) pyridine (10.00 g, 41.663 mmol, 1.00 equiv), Pd(DtBPF)Cl₂ (1.00 g, 4.1663 mmol, 0.10 equiv) in 500 mL EtOH was added TEA (5.00 g, 83.326 mmol, 2.00 equiv) in a pressure tank. The mixture was purged with nitrogen for 10 min and then was pressurized to 30 atm with carbon monoxide at 100° C. and stirred overnight. The reaction mixture was cooled to room temperature and filtered to remove insoluble solids. The resulting mixture was diluted with water (1 L). The resulting mixture was extracted with EtOAc (5×200 mL). The resulting mixture was concentrated under reduced pressure. The residue was purified by silica gel column chromatography, eluted with PE/EtOAc (50:1) to afford I-2a (7.1 g, 73.08%) as a brown liquid.

### Synthesis of I-2b

To a stirred solution of 1-2a (3.50 g, 15.009 mmol, 1.00 equiv) and SeO₂ (3.33 g, 30.018 mmol, 2.00 equiv) was added dioxane (400.00 mL) at room temperature under air atmosphere. The resulting mixture was stirred overnight at 110° C. The reaction was quenched with water at room temperature. The aqueous layer was extracted with EtOAc (2×200 mL). The residue was purified by silica gel column chromatography, eluted with PE/EtOAc (5:1) to afford I-2b (3 g, 73.59%) as an off-white solid.

### Synthesis of I-2c

Into a 250 mL 3-necked round-bottom flask were added 1-2b (3.00 g, 0.012 mmol, 1.00 equiv), AcOH (6.40 mL), H₂SO₄ (0.50 mL) and CH(OMe)₃ (40.00 mL) at room temperature. The resulting mixture was stirred for an additional 30 min at 50° C. The resulting mixture was stirred for overnight at room temperature under air atmosphere. The reaction was quenched with water/NaHCO₃ at room temperature. The aqueous layer was extracted with EtOAc (2×40 mL). The residue was purified by silica gel column chromatography, eluted with PE/EtOAc (5:1) to afford I-2c 2.8 g (75.52%) as an off-white solid.

### Synthesis of I-2d

Into a 100 mL 3-necked round-bottom flask were added 1-2c (2.80 g, 9.548 mmol, 1.00 equiv), MeOH (30.00 mL) at room temperature. To the above mixture was added NaBH₄ (5.01 g, 18.096 mmol, 2 equiv) at 0° C. The resulting mixture was stirred for 2h at 0° C. under air atmosphere. The reaction was quenched with NH₄Cl (aq.) at room temperature. The aqueous layer was extracted with EtOAc (3×20 mL). The resulting mixture was concentrated under reduced pressure. The residue was purified by silica gel column chromatography, eluted with PE/EtOAc (6:1) to afford I-2d (1.2 g, 40.02%) as an off-white solid.

### Synthesis of I-2e

Into a 50 mL 2-necked round-bottom flask were added I-2d (L 10 g, 4.379 mmol, 1.00 equiv), MnO₂ (5.71 g, 65,685 mmol, 15.00 equiv) and DCM (15.00 mL) at room temperature. The resulting mixture was stirred overnight at 40° C. under air atmosphere. The reaction was quenched with water (15 mL) at room temperature. The aqueous layer was extracted with EtOAc (3×10 mL). The residue was purified by silica gel column chromatography, eluted with PE/EtOAc (5:1) to afford I-2e (500 mg, 41.24%) as an off-white solid.

### Synthesis of I-2f

Into a 20 mL vial were added I-2e (500.00 mg, 2.007 mmol, 1.00 equiv), (3S)-3-methylpiperidine (398.00 mg, 4.013 mmol, 2 equiv), TEA (406.08 mg, 4.014 mmol, 2.00 equiv) and DCE (6 ml), NaBH(OAc)₃ (1275.78 mg 6.021 mmol, 3.00 equiv) at room temperature. The resulting mixture was stirred for 2 h at room temperature under air atmosphere. The reaction was quenched with water at room temperature. The aqueous layer was extracted with EtOAc (3×10 mL). The residue was purified by prep-TLC (PE/EtOAc 5:1) to afford I-2f (500 mg, 71.23%) as an off-white solid.

### Synthesis of I-2

Into a 20 mL vial were added I-2f (500.00 mg, 1,504 mmol, 1.00 equiv), H₂O (5.50 mL) and HCl (0.50 mL) at room temperature. The resulting mixture was stirred overnight at 80° C. under air atmosphere. The reaction was quenched with NaHCO₃ (aq.) at room temperature. The aqueous layer was extracted with EtOAc (2×5 mL). The residue was purified by silica gel column chromatography, eluted with PE/EtOAc (2:1) to afford I-2 (300 mg, 55.72%) as a yellow oil.

Alternatively, I-2 Can also be made as follows:

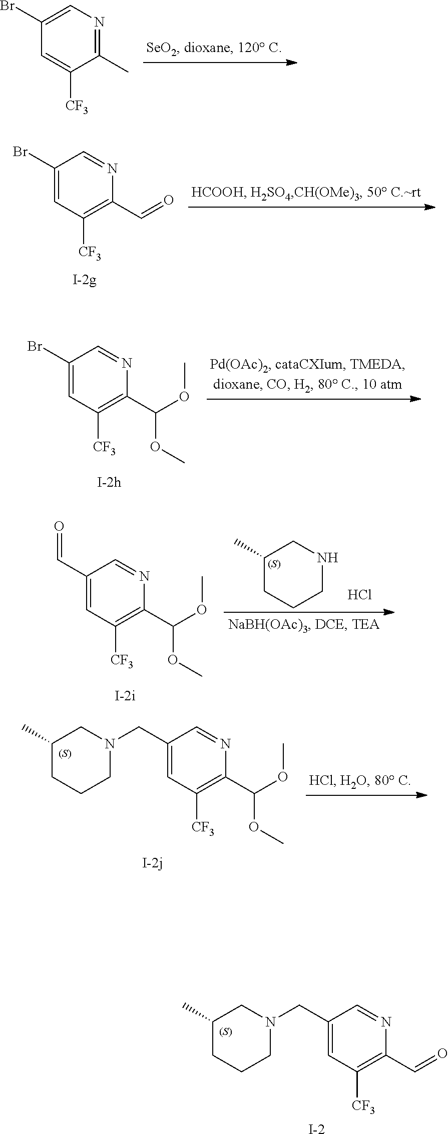

### Synthesis of I-2g

A solution of 5-bromo-2-methyl-3-(trifluoromethyl)pyridine (60 g, 249.976 mmol, 1 equiv) in dioxane (350 mL) was added SeO₂ (69.35 g, 624.940 mmol, 2.5 equiv). The resulting mixture was stirred overnight at 120 degrees C. The resulting mixture was filtered, the filter cake was washed with EtOAc (3×50 mL). The filtrate was diluted with water (300 mL). The aqueous layer was extracted with EtOAc (3×100 ml). The residue was purified by silica gel column chromatography, eluted with PE/EA (50:1) to afford I-2g (49 g, 69.45%) as a yellow oil.

### Synthesis of I-2h

Into a 250 mL round-bottom flask were added 1-2g (10 g 40.48 mmol, 1.00 equiv) and CH(OMe)3 (100 mL) at room temperature. To the above mixture was added HCOOH (3 mL) and H2SO4 (1 mL) at room temperature. The resulting mixture was stirred overnight at 50 degrees C. The reaction was quenched by the addition of NaHCO₃ (aq.) (300 mL) at room temperature. The aqueous layer was extracted with EtOAc (3×100 mL). The resulting mixture was concentrated under vacuum. The residue was purified by silica gel column chromatography, eluted with PE/EA (5:1) to afford I-2h (8 g 67.45%) as yellow oil.

### Synthesis of I-2i

To a solution of 12h (8 g, 26.660 mmol, 1 equiv) in 100 mL dioxane was added Pd(OAc)2 (0.60 g, 2.666 mmol, 0.1 equiv) in a pressure tank. The mixture was purged with nitrogen and then was pressurized to 10 atm with carbon monoxide/hydrogen (1:1) at 80° C. overnight. The reaction mixture was cooled to room temperature and diluted with water (600 mL). The aqueous layer was extracted with EtOAc (3×300 mL). The resulting mixture was concentrated under vacuum. The residue was purified by silica gel column chromatography, eluted with PE/EA (10:1) to afford I-2i (5 g, 60.21%) as a brown oil.

### Synthesis of I-2j

Into a 100 ml, round-bottom flask were added 1-2i (1.9 g, 7.625 mmol, 1.00 equiv), (3S)-3-methylpiperidine hydrochloride (1.24 g, 9.150 mmol, 1.2 equiv), DCE (30 mL) and Et3N (0.93 g, 9.150 mmol, 1.2 equiv) at room temperature. The mixture was stirred for 10 min at room temperature. To the above mixture was added NaBH(OAc)3 (4.85 g, 22.875 mmol, 3 equiv) at room temperature. The resulting mixture was stirred overnight at room temperature. The reaction was quenched with NH4Cl (aq.) (100 mL) at room temperature. The aqueous layer was extracted with DCM (3×40 mL). The resulting mixture was concentrated under vacuum. The residue was purified by silica gel column chromatography, eluted with PE/EA (3:1) to afford I-2j (1.7 g, 61.04%) as brown oil.

### Synthesis of I-2

Into a 50 mL round-bottom flask were added 1-2j (1.7 g, 5,115 mmol, 1.00 equiv) and 1 M HCl (20 mL) at room temperature. The resulting mixture was stirred for 3 h at 80° C. The residue was basified to pH 7 with NH4HCO3 (aq.). The aqueous layer was extracted with EtOAc (3×50 mL). The resulting mixture was concentrated under vacuum. The residue was purified by silica gel column chromatography, eluted with PE/EA (10:1) to afford Compound 1-2 (1.5 g, 94.24%) as colorless oil.

Alternatively, I-2g can also be made as follows:

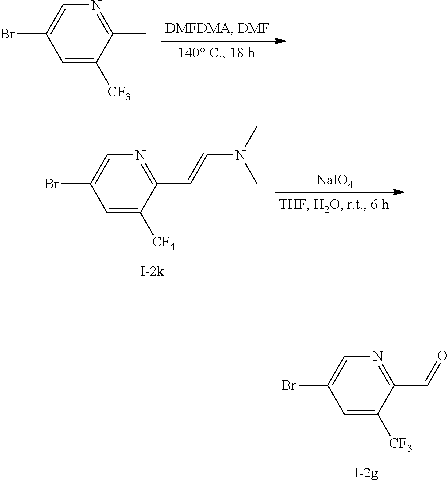

### Synthesis of I-2k

A mixture of 5-bromo-2-methyl-3-(trifluoromethyl)pyridine (25 g, 104.16 mmol, 1 eq) in DMF (300 mL) and DMF-DMA (269.10 g, 2.26 mol, 300 mL) was stirred at 140° C. for 18 hr. The reaction mixture was concentrated in vacuum to afford I-2k (30 g, crude) as a brown oil, which was used directly without further purification.

### Synthesis of I-2g

To a solution of I-2k (30 g, 101 mmol) in THF (150 mL) and water (150 mL) was added NaIO4 (65.2 g, 304 mmol). The mixture was stirred at 20° C. for 6 hr. The reaction mixture was filtered and the filter cake was washed with ethyl acetate (200 mL). The filtrate was washed with saturated aqueous sodium bicarbonate solution (200 mL) and saturated aqueous brine solution (200 mL), dried over anhydrous sodium sulfate, filtered, and concentrated in vacuum. The residue was purified by normal phase SiO2 chromatography (0-20% EtOAc/petroleum ether) to afford I-2g (8 g, 30.9% yield) as a brown oil.

### Intermediate I-3: Synthesis of Intermediate 3

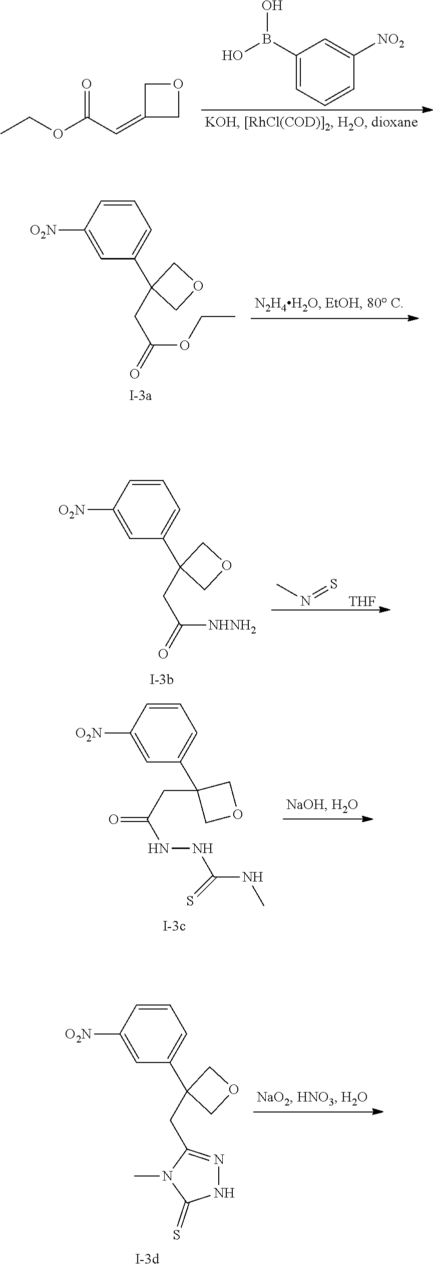

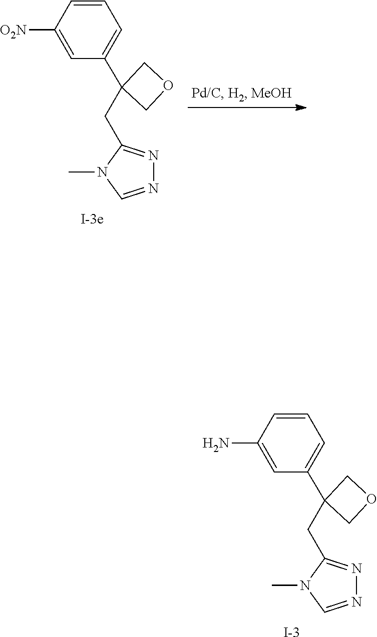

### Synthesis of I-3a

To a stirred solution of KOH (23.68 g, 422.077 mmol, 1.2 equiv) in H₂O (285.00 mL) and dioxane (1000.00 mL) were added [Rh(COD)Cl]₂ (4.00 g, 8.112 mmol, 0.02 equiv) in portions at room temperature under nitrogen atmosphere. The resulting mixture was stirred for 1 h at room temperature under nitrogen atmosphere. To the above mixture were added ethyl 2-(oxetan-3-ylidene)acetate (50.00 g, 351.731 mmol, 1.00 equiv) and 3-nitrophenylboronic acid (117.43 g, 703.462 mmol, 2 equiv) in portions at room temperature. The resulting mixture was stirred for an additional 16 h at room temperature under nitrogen atmosphere. The reaction was quenched by the addition of NH₄Cl (aq.) (3 L) at room temperature. The aqueous layer was extracted with EtOAc (3×5 L). The resulting mixture was concentrated under reduced pressure. The residue was purified by silica gel column chromatography, eluted with PE/EtOAc (10:1) to afford I-3a (41 g, 73.24%) as a yellow solid.

### Synthesis of I-3b

A mixture of I-3a (30.00 g, 113.094 mmol, 1.00 equiv) in EtOH (150 mL) and hydrazine hydrate (98%) (45.29 g, 904.756 mmol, 8 equiv) was stirred for 24 h at 80° C. The mixture was allowed to cool down to room temperature. The resulting mixture was diluted with water (750 mL). The aqueous layer was extracted with CH₂Cl₂/MeOH (10/1) (5×1 L). The resulting oil was dried with anhydrous sodium sulfate. The resulting mixture was filtered, and the filter cake was washed with MeOH (3×100 mL). The filtrate was concentrated under reduced pressure to afford I-3b (26.0 g, crude) as a yellow oil.

### Synthesis of I-3c

To a stirred solution of I-3b (26.00 g, 103.486 mmol, 100 equiv) in tetrahydrofuran (260.00 mL) was added methyl isothiocyanate (15.13 g, 206.972 mmol, 2.00 equiv) at room temperature. The resulting mixture was stirred for 4 h at room temperature. The resulting mixture was diluted with water (600 mL). The precipitated solids were collected by filtration and washed with water (3×50 mL) to afford I-3c (35.0 g) as a yellow solid.

### Synthesis of I-3d

To a stirred solution of I-3c (35 g, 107.905 mmol, 1.00 equiv) was added NaOH (864 mL, 863.240 mmol, 8.00 equiv, 1 M) at room temperature. The resulting mixture was stirred for 2 h at room temperature. The resulting mixture was diluted with water (1 L). The mixture was acidified to pH 5 with HC (1 M). The aqueous layer was extracted with CH₂Cl₂/MeOH (10/1) (3×2 L). The resulting mixture was concentrated under reduced pressure to afford I-3d (24 g) as a yellow solid.

### Synthesis of I-3e

To a stirred solution of I-3d (24.00 g, 78.344 mmol, 1.00 equiv) and NaNO₂ (54.05 g, 783.443 mmol, 10.00 equiv) in H₂O (150.00 mL) and ethyl acetate (50.0 mL) was added HNO₃ (500 mL, 783.443 mmol, 10.00 equiv, 1 M) dropwise at 0° C. The resulting mixture was stirred overnight at 0° C. The reaction was quenched by the addition of NaHCO₃ (aq.) (1 L) at room temperature. The aqueous layer was extracted with CH₂Cl₂/MeOH (10/1) (3×2 L), The resulting mixture was concentrated under reduced pressure to afford I-3e (19 g) as a yellow solid.

### Synthesis of I-3

To a solution of I-3e (19.00 g) in 190 mL MeOH was added Pd/C (30%, 5.7 g) under nitrogen atmosphere in a 500 nil, round-bottom flask. The mixture was hydrogenated at room temperature for 4 h under hydrogen atmosphere using a hydrogen balloon, filtered through a Celite pad and concentrated under reduced pressure. This resulted in I-3 (16 g) as a yellow solid.

### Example 1. Synthesis of Compound 1

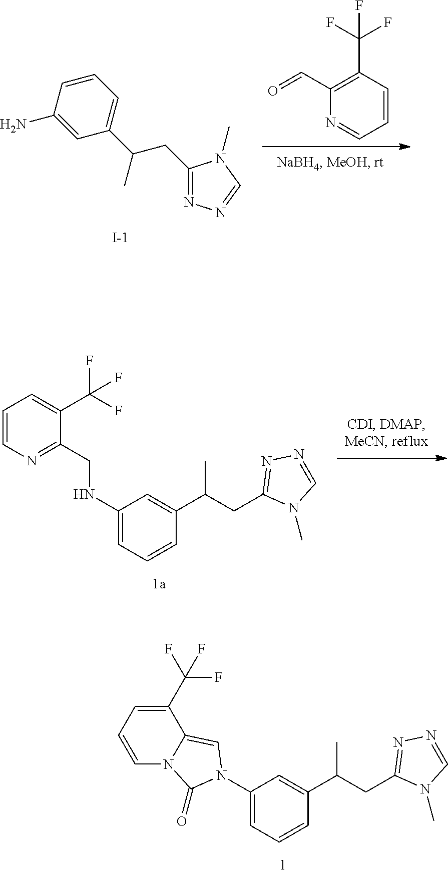 [SMILES: CC(Cc1nncn1C)c1cccc(N)c1 ; O=Cc1ncccc1C(F)(F)F ; [I-] ; CC(Cc1nncn1C)c1cccc(NCc2ncccc2C(F)(F)F)c1 ; CC(Cc1nncn1C)c1cccc(-n2cc3c(C(F)(F)F)cccn3c2=O)c1]

### Synthesis of 1a

To a stirred solution of I-1 (300.00 mg, 1.387 mmol, 1.00 equiv) in MeOH (4.00 mL) was added 3-(trifluoromethyl)pyridine-2-carbaldehyde (242.88 mg, 1.387 mmol, 1.00 equiv) at room temperature under air atmosphere. The resulting mixture was stirred overnight at room temperature. To the above mixture was added NaBH₄ (104.95 mg, 2.774 mmol, 2.00 equiv) at 0° C. The resulting mixture was stirred for an additional 1 h at room temperature. The reaction was quenched by the addition of NH₄Cl (aq.) (30 mL) at room temperature. The aqueous layer was extracted with EtOAc (15 mL). The resulting mixture was concentrated under reduced pressure. The residue was purified by prep-TLC (CH₂Cl₂/MeOH=30:1) to afford 1 a (200 mg, 38.41%) as a yellow solid.

### Synthesis of 1

To a stirred mixture of 1a (180.00 mg, 0.479 mmol, 1.00 equiv) and CDI (116.62 mg, 0.718 mmol, 1.50 equiv) in CH₃CN (3.00 mL) was added DMAP (117.16 mg, 0.958 mmol, 2.00 equiv) at room temperature under air atmosphere. The resulting mixture was stirred for 2 h at 90° C. The resulting mixture was diluted with water (100 mL). The aqueous layer was extracted with EtOAc (30 mL). The resulting mixture was concentrated under reduced pressure. The residue was purified by prep-TLC (CH₂Cl₂/MeOH=20:1) to afford Compound 1 (80 mg) as a yellow solid. The crude product (80 mg) was purified by prep-HPLC with the following conditions (Column: XBridge Prep OBD C18 Column, 30*150 mm, 5 μm; Mobile Phase A: water (10 mmol/L NH₄HCO₃+0.1% NH₃·H₂O), Mobile Phase B: MeOH; Flow rate: 60 mL/min; Gradient: 41% B to 71% B in 8 min; Wave Length: 254; 220 nm; RT1 (min): 717) to afford Compound 1 (30.2 mg) as a yellow solid.

LCMS: (ES, m/z): [M+H]⁺ 401.

¹H NMR: (300 MHz, DMSO-d6, ppm): δ 1.30-1.33 (d, 3H), 3.01-3.04 (m, 2H), 3.32-3.35 (m, 1H), 3.46 (s, 3H), 6.26-6.31 (m, 1H), 7.08-7.10 (m, 1H), 7.23-7.25 (m, 1H), 7.33 (s, 1H), 7.42-7.44 (m, 1H), 7.70-7.73 (m, 2H), 7.78-7.81 (d, 1H), 8.28 (s, 1H).

### Example 2. Synthesis of Compound 2

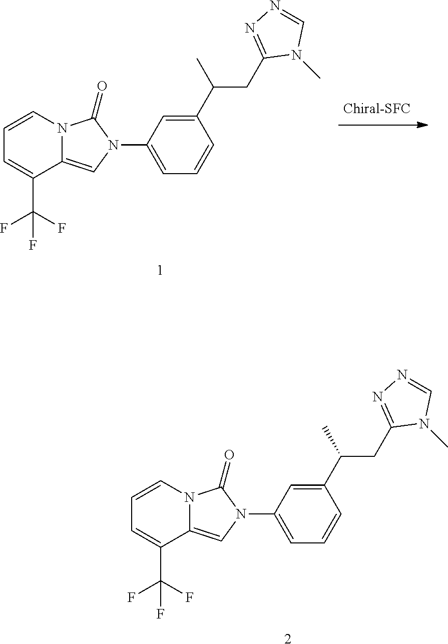 [SMILES: CC(Cc1nncn1C)c1cccc(-n2cc3c(C(F)(F)F)cccn3c2=O)c1 ; "1" ; C[C@H](Cc1nncn1C)c1cccc(-n2cc3c(C(F)(F)F)cccn3c2=O)c1 ; "2"]

### Synthesis of 2

The Compound 1 (340 ng) was separated by prep-chiral HPLC with the following conditions (Column: CHIRALPAK IH, 2*25 cm, 5 μm; Mobile Phase A: Hex (0.5% 2M NH₃-MeOH), Mobile Phase B: EtOH; Flow rate: 20 mL/min; Gradient: 25% B to 25% B in 21.5 min; Wave Length: 254/220 nm; RT1 (min): 14.79; Sample Solvent: EtOH; Injection Volume: 0.4 mL; Number Of Runs: 18) to afford Compound 2 (92.4 mg) as a yellow solid.

LCMS: (ES, m/z): [M+H]⁺ 402.

¹H NMR: (400 MHz, DMSO-d6, ppm): δ 1.36-1.38 (d, 3H), 3.02-3.19 (m, 2H), 3.35-3.40 (m, 1H), 3.43 (s, 3H), 6.31-6.35 (m, 1H), 7.01-7.03 (m, 1H), 7.10 (s, 1H), 7.22-7.24 (m, 1H), 7.40-7.45 (m, 1H), 7.53-7.58 (m, 2H), 7.73-7.76 (m, 1H), 8.24 (s, 1H).

### Example 3. Synthesis of Compound 3

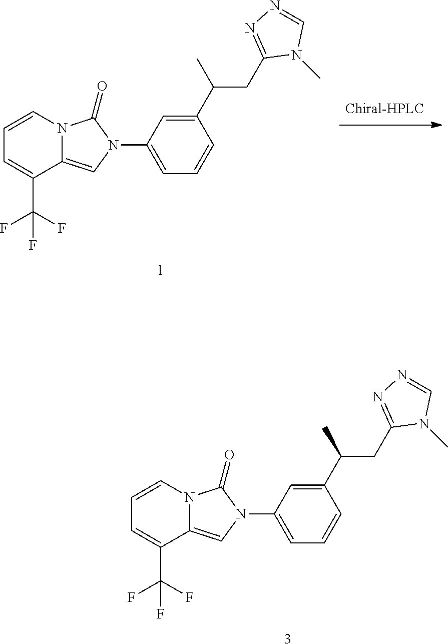 [SMILES: CC(Cc1nncn1C)c1cccc(-n2cc3c(C(F)(F)F)cccn3c2=O)c1 ; "1" ; C[C@@H](Cc1nncn1C)c1cccc(-n2cc3c(C(F)(F)F)cccn3c2=O)c1 ; "3"]

### Synthesis of 3

Compound 1 (340 ng) was purified by prep-chiral HPLC with the following conditions (Column: CHIRALPAK IH, 2*25 cm, 5 μm; Mobile Phase A: Hex (0.5% 2M NH₃-MeOH), Mobile Phase B: EtOH; Flow rate: 20 mL/min; Gradient: 25% B to 25% B in 215 min; Wave Length: 254/220 nm; RT2 (min): 18.83; Sample Solvent: EtOH; Injection Volume: 0.4 mL; Number Of Runs: 18) to afford Compound 3 (92.0 mg) as a yellow solid.

LCMS: (ES, m/z): [M+H]⁺ 402.

¹H NMR: (400 MHz, DMSO-d6, ppm): δ 1.36-1.38 (d, 3H), 3.02-3.19 (m, 2H), 3.35-3.40 (m, 1H), 3.43 (s, 3H), 6.31-6.35 (m, 1H), 7.01-7.03 (m, 1H), 7.10 (s, 1H), 7.22-7.24 (m, 1H), 7.40-7.45 (m, 1H), 7.53-7.58 (m, 2H), 7.73-7.76 (m, 1H), 8.24 (s, 1H).

### Example 4 Synthesis of Compound 4

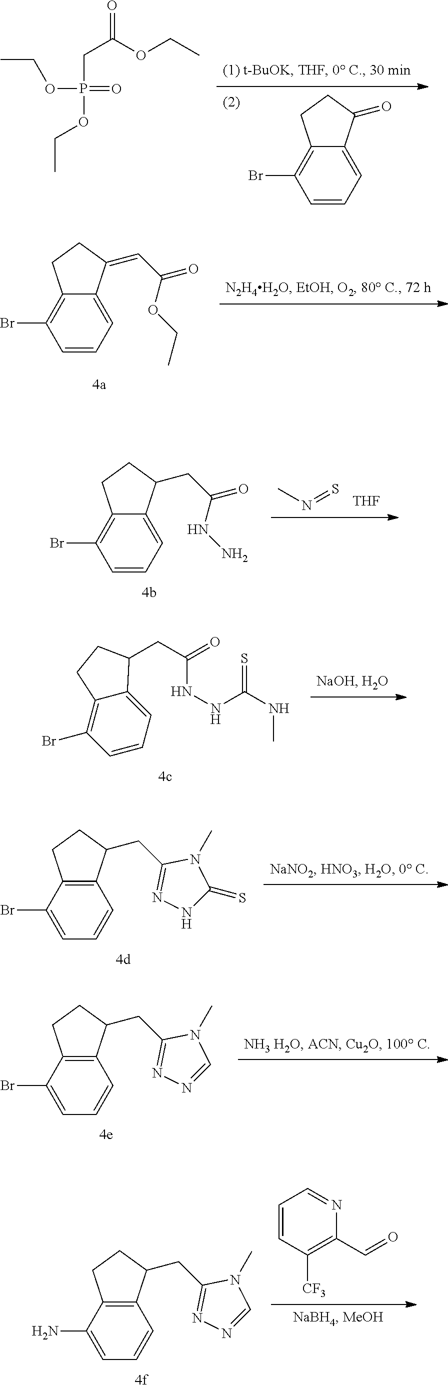

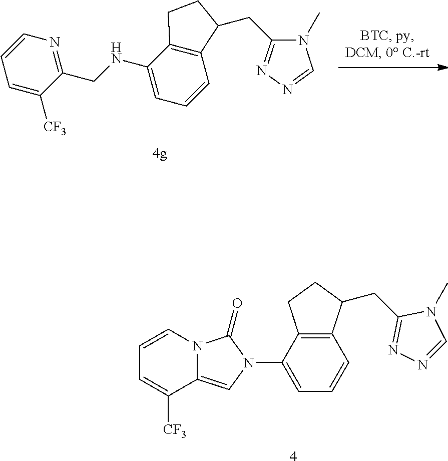 [SMILES: Cn1cnnc1CC1CCc2c(NCc3ncccc3C(F)(F)F)cccc21 ; "BTC, py,DCM, 0° C.-rt" ; "4g" ; Cn1cnnc1CC1CCc2c1cccc2-n1cc2c(C(F)(F)F)cccn2c1=O ; "4"]

### Synthesis of 4a

A solution of triethyl phosphonoacetate (10.00 g, 44.604 mmol, 1.00 equiv) and t-BuOK (10.01 g, 0.089 mmol, 2 equiv) in THF (100 mL) was stirred for 30 min at 0° C. under nitrogen atmosphere. To the above mixture was added 4-bromo-2,3-dihydroinden-1-one (9.41 g, 0.045 mmol, 1 equiv) in THF (20 mL) dropwise over 20 min at 0° C. The resulting mixture was stirred for an additional 3 h at 0° C. The reaction was quenched by the addition of NH₄Cl (aq.) (150 mL) at 0° C. The resulting mixture was extracted with EtOAc (3×100 mL). The combined organic layers were washed with water (30 mL), dried over anhydrous Na₂SO₄. After filtration, the filtrate was concentrated under reduced pressure. The residue was purified by silica gel column chromatography, eluted with PE/EtOAc (5:1) to afford 4a (4.4 g, 34.03%) as a light yellow oil.

### Synthesis of 4b

Into a 250 mL 3-necked round-bottom flask were added 4a (4.40 g, 15.650 mmol, 1.00 equiv), EtOH (70.00 mL) and hydrazine hydrate (7.83 g, 156.500 mmol, 10.00 equiv) at room temperature. The resulting mixture was stirred for 72 h at 80° C. under oxygen atmosphere. The resulting mixture was concentrated under reduced pressure. The resulting mixture was diluted with EtOAc (100 mL), washed with water (20 mL), dried over anhydrous Na₂SO₄. After filtration, the filtrate was concentrated under reduced pressure. The crude product was used in the next step directly without further purification. This resulted in 4b (4.0 g, 83.57%) as a light yellow oil.

### Synthesis of 4c

Into a 250 ml, 3-necked round-bottom flask were added 4b (4.00 g, 14.862 mmol, 1.00 equiv), tetrahydrofuran (50.00 mL) and methyl isothiocyanate (2.17 g, 29.681 mmol, 2.00 equiv) at room temperature. The resulting mixture was stirred for 5 h at room temperature under nitrogen atmosphere. The reaction was quenched with water (100 mL) at room temperature. The resulting mixture was filtered; the filter cake was washed with water (3×5 mL). This resulted in 4c (4.5 g, 88.47%) as an off-white solid.

### Synthesis of 4d

Into a 250 ml, 3-necked round-bottom flask were added 4c (4.50 g, 13.148 mmol, 1.00 equiv), H₂O (50.00 ml) and NaOH (0.53 g, 0,000 mmol, 1.00 equiv) at room temperature. The resulting mixture was stirred for 3 h at room temperature. The reaction was quenched by the addition of NH₄Cl (aq.) (20 ml) at room temperature. The resulting mixture was filtered; the filter cake was washed with water (3×5 mL). This resulted in 4d (3 g, 63.33%) as an off-white solid.

### Synthesis of 4e

Into a 500 ml, 3-necked round-bottom flask were added 4d (2.50 g, 7.710 mmol, 1.00 equiv), ethyl acetate (50.00 mL) and NaNO₂ (5.32 g, 77.100 mmol, 10.00 equiv) at 0° C. To the above mixture was added HNO₃ (4.86 g, 77.127 mmol, 10.00 equiv) in 1120 (150.00 mL) dropwise over 0.5 h at 0° C. The resulting mixture was stirred for 5 h at 0° C. under nitrogen atmosphere. The reaction was quenched by the addition of ice/salt (100 mL) at 0° C. The resulting mixture was extracted with EtOAc (3×100 mL). The combined organic layers were washed with water (30 mL), dried over anhydrous Na₂SO₄. After filtration, the filtrate was concentrated under reduced pressure. The residue was purified by silica gel column chromatography, eluted with CH2Cl₂/MeOH (30:1) to afford 4e (1.6 g, 63.92%) as a light yellow oil.

### Synthesis of 4f

Into a 250 mL sealed tube were added 4e (500.00 mg, 1.711 mmol, 1.00 equiv), NH₃·H₂O (70.00 mL), CH₃CN (70.00 mL) and Cu₂O (48.97 mg, 0.342 mmol, 0.20 equiv) at room temperature. The resulting mixture was stirred for 12 h at 100° C. under nitrogen atmosphere. The resulting mixture was concentrated under reduced pressure. The residue was purified by prep-TLC (CH₂Cl₂/MeOH 10:1) to afford 4f (270 mg, 67.04%) as light yellow solid.

### Synthesis of 4g

Into a 100 ml, 3-necked round-bottom flask were added 4f (230.00 mg, 1,007 mmol, 1.00 equiv), MeOH (5.00 mL and 3-(trifluoromethyl)pyridine-2-carbaldehyde (264.62 mg, 1,511 mmol, 1.50 equiv) at room temperature. The resulting mixture was stirred for 12 h at room temperature under nitrogen atmosphere. To the above mixture was added NaBH₄ (76.23 mg, 2.014 mmol, 2.00 equiv) in portions over 10 min at 0° C. The resulting mixture was stirred for additional 2 h at 0° C. The reaction was quenched by the addition of sat. NH₄Cl (aq.) (20 mL) at 0° C. The resulting mixture was extracted with EtOAc (3×20 mL). The residue was purified by prep-TLC (CH₂Cl₂/MeOH 10:1) to afford 4g (130 mg, 30.98%) as a light yellow solid.

### Synthesis of 4

Into a 8 mL sealed tube were added 4g (1301.00 mg, 0,336 mmol, 1.00 equiv), DCM (2.00 mL), pyridine (159.26 mg, 2.016 mmol, 6.00 equiv) and triphosgene (39.83 mg, 0.134 mmol, 0.40 equiv) at 0° C. The resulting mixture was stirred for 3 h at 0° C. under nitrogen atmosphere. The resulting mixture was concentrated under reduced pressure. The residue was purified by prep-TLC (CH₂Cl₂/MeOH 10:1) to afford crude product (120 mg). The crude product (120 ng) was purified by prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 22% B to 50% B in 8 min, 50% B; Wave Length: 220 nm; RT1 (min): 7.32) to afford Compound 4 (53.2 mg, 37.97%) as a yellow solid.

LCMS: (ES, m/z): [M+H]⁺ 414

¹H NMR: (400 MHz, DMSO-d6, ppm): δ 1.76-1.83 (m, 1H), 2.24-2.32 (m, 1H), 2.78-2.92 (m, 3H), 3.20-3.34 (m, 1H), 3.69 (s, 3H), 3.71-3.73 (m, 1H), 6.27-6.31 (m, 1H), 7.08-7.14 (m, 2H), δ 7.30-7.35 (m, 3H), δ 7.79-7.81 (d, 1H), 38.34 (m, 1H).

### Example 5. Synthesis of Compound 5

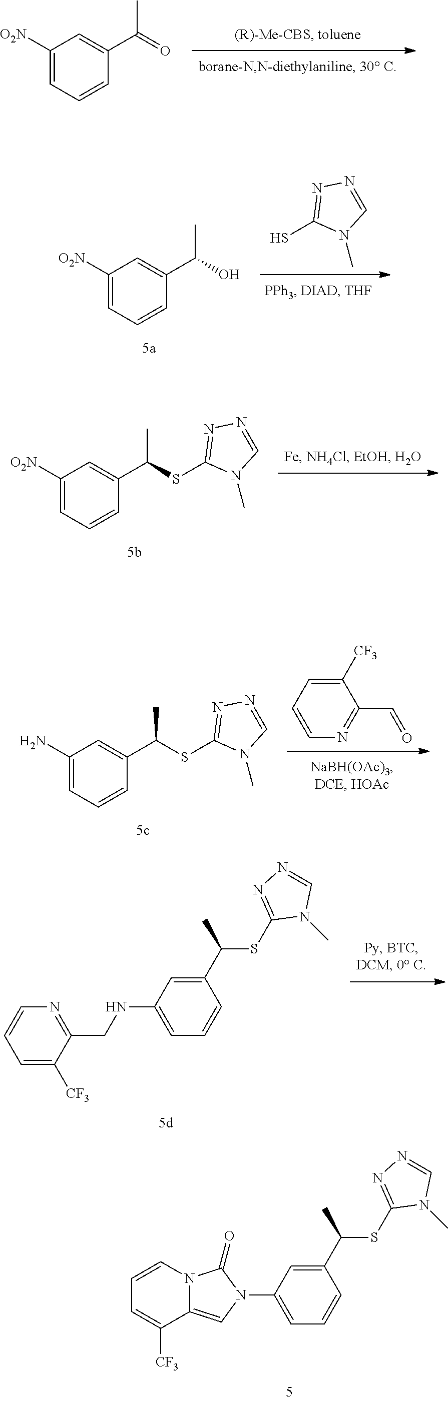

### Synthesis of 5a

A solution of borane-N,N-diethylaniline (1.99 g, 13,321 mmol, 1.10 equiv) and (R)-Me-CBS (12.00 mL, 12.0 mmol, 1.00 equiv, 1M in toluene) in toluene (50.00 mL) was stirred for 20 min at 30° C. under nitrogen atmosphere. Then 3-nitroacetophenone (2.00 g, 12.110 mmol, 1.00 equiv) was added at room temperature. The reaction was washed with water (20 mL) at room temperature. The resulting mixture was concentrated under reduced pressure. The residue was purified by silica gel column chromatography, eluted with (PE/EtOAc 2:1) to afford 5a (1.7 g, 80.62%) as a white solid.

### Synthesis of 5b

To a stirred mixture of 5a (1.50 g, 8.973 mmol, 1.00 equiv), 4-methyl-1,2,4-triazole-3-thiol (1.24 g, 10.769 mmol, 1.20 equiv) and PPh₃ (4.71 g, 17,946 mmol, 2 equiv) in THF (25.00 mL) was added DIAD (2.72 g, 13.460 mmol, 15 equiv) dropwise at 0° C. under nitrogen atmosphere. The reaction was quenched with water (20 mL) at room temperature. The resulting mixture was extracted with EtOAc (2×30 mL). The combined organic layers were washed with brine (20 mL), dried over anhydrous Na₂SO₄. After filtration, the filtrate was concentrated under reduced pressure. The residue was purified by silica gel column chromatography, eluted with (CH₂Cl₂/MeOH 25:1) to afford 5b (1.8 g, 70%) as a yellow solid.

### Synthesis of 5c

A mixture of 5b (180 g, 6.810 mmol, 1.00 equiv), Fe (1.14 g, 20.431 mmol, 3 equiv) and NH₄Cl (2.19 g, 40.863 mmol, 6 equiv) in EtOH (20.00 mL) and H₂O (5.00 mL) was stirred for 2 h at 80° C. under nitrogen atmosphere. The mixture was allowed to cool down to room temperature. The resulting mixture was filtered with filter paper, the filter cake was washed with EtOAc (3×5 mL). The resulting mixture was diluted with water (50 mL) and extracted with EtOAc (3×30 mL). The combined organic layers were dried over anhydrous Na₂SO₄ and concentrated under reduced pressure. This resulted in 5c (1.5 g, 87.42%) as a yellow solid.

### Synthesis of 5d

A mixture of 5c (800.00 mg, 3.414 mmol, 1.00 equiv), 3-(trifluoromethyl)pyridine-2-carbaldehyde (896.77 mg, 5.121 mmol, 1.50 equiv), NaBH(OAc)₃ (2170.79 mg, 10.242 mmol, 3 equiv) and HOAc (1025.13 mg, 17.071 mmol, 5 equiv) in DCE (7.00 mL) was stirred for 2 h at room temperature under nitrogen atmosphere. The resulting mixture was washed with water (10 mL), dried over anhydrous Na₂SO₄. After filtration, the filtrate was concentrated under reduced pressure. The residue was purified by prep-TLC (PE/EtOAc 1:1) to afford 5d (540 mg, 37.79%) as a yellow solid.

### Synthesis of 5

To a stirred mixture of 5d (350.00 mg, 0.890 mmol, 1.00 equiv) and pyridine (422.21 mg, 5.338 mmol, 6.00 equiv) in DCM (4.00 mL) was added triphosgene (92.40 mg, 0.311 mmol, 0.35 equiv) dropwise at 0° C. under nitrogen atmosphere. The reaction was washed with water (10 mL) at room temperature. The residue was purified by prep-TLC (CH₂Cl₂/MeOH 15:1) to afford Compound 5 (53.1 mg, 13.79%) as an orange solid,

LCMS: (ES, m/z): [M+H]⁺ 420

¹H NMR: (400 MHz, DMSO-d6, δ ppm): δ 1.72-1.76 (d, 3H), 3.48 (s, 3H), 6.09-6.13 (d, 1H), 6.27-6.30 (m, 1H), 7.09-7.10 (d, 1H), 7.27-7.29 (d, 1H), 7.33 (s, 1H), 7.48-7.50 (m, 1H), 7.73-7.75 (d, 1H), 7.78-7.80 (d, 1H), 7.87 (s, 1H), 8.55 (s, 1H).

### Example 6. Synthesis of Compound 6

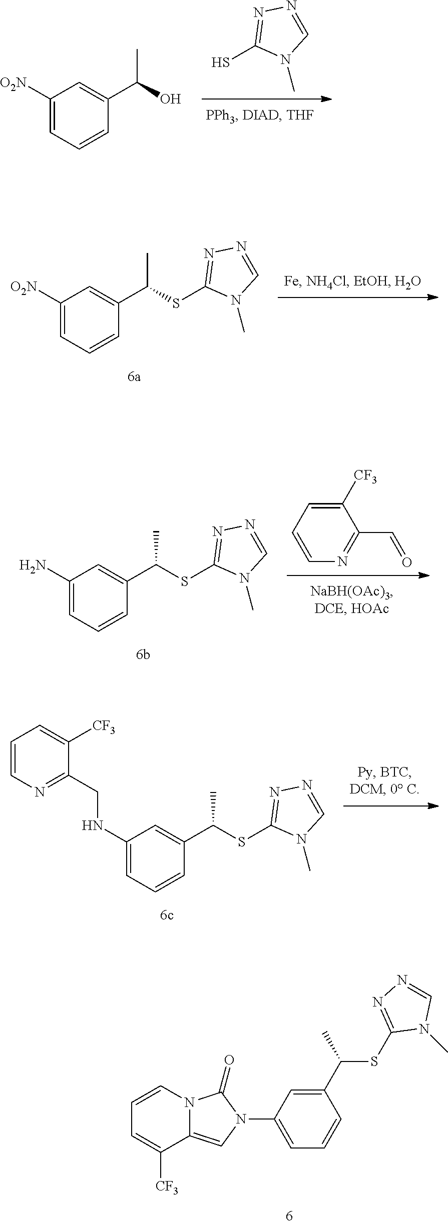

### Synthesis of 6a

To a stirred mixture of (1R)-1-(3-nitrophenyl)ethanol (2.00 g, 11,964 mmol, 1.00 equiv), 4-methyl-1,2,4-triazole-3-thiol (1.65 g, 14.329 mmol, 1.20 equiv) and PPh₃ (6.28 g, 23.929 mmol, 2 equiv) in THE (25.00 mL) were added DIAD (3.63 g, 17.946 mmol, 1.5 equiv) dropwise at 0° C. under nitrogen atmosphere. The resulting mixture was diluted with brine (100 mL). The resulting mixture was extracted with EtOAc (2×50 mL). The combined organic layers were washed with brine (10 mL), dried over anhydrous Na₂SO₄. After filtration, the filtrate was concentrated under reduced pressure. The residue was purified by prep-TLC (CH₂Cl₂/MeOH 25:1) to afford 6a (1.6 g, 49.59%) as a yellow solid.

### Synthesis of 6b

A mixture of 6a (1.60 g, 6.054 mmol, 1.00 equiv), Fe (1.01 g, 18.161 mmol, 3 equiv) and NH₄Cl (1.94 g, 36.322 mmcl, 6 equiv) in EtOH (20.00 ml) and H₂O (4.00 mL) was stirred for 2 h at 80° C. under nitrogen atmosphere. The mixture was allowed to cool down to room temperature. The resulting mixture was diluted with EtOAc (20 mL). The resulting mixture was filtered, the filter cake was washed with EtOAc (3×10 ml). The combined organic layers were washed with brine (3×4 mL), dried over anhydrous Na₂SO₄. After filtration, the filtrate was concentrated under reduced pressure. This resulted in 6b (610 mg, 41.71%) as a yellow solid.

### Synthesis of 6c

A mixture of 6b (610.00 mg, 2.603 mmol, 1.00 equiv), 3-(trifluoromethyl) pyridine-2-carbaldehyde (683.79 ng, 3.905 mmol, 1.5 equiv), NaBH(OAc)3 (1655.22 ng, 7,810 mmcl, 3 equiv) and HOAc (781.66 mg, 13.016 mmol, 5 equiv) in DCE (6.50 mL) was stirred for 2 h at 80° C. under nitrogen atmosphere. The mixture was allowed to cool down to room temperature. The resulting mixture was diluted with DCM (10 mL). The resulting mixture was washed with water (10 mL). The residue was purified by prep-TLC (CH₂Cl₂/MeOH 25:1) to afford 6c (375 mg, 35.52%) as a yellow solid.

### Synthesis of 6

To a stirred mixture of 6c (280.00 mg, 0.712 mmol, 1.00 equiv) and pyridine (337.77 mg 4:270 mmol, 6 equiv) in DCM (3.00 mL) was added triphosgene (73.92 mg, 0.249 mmol, 0.35 equiv) dropwise at 0° C. under nitrogen atmosphere. The reaction was quenched with water at room temperature. The resulting mixture was extracted with DCM (3×5 mL). The residue was purified by prep-TLC (CH₂Cl₂/MeOH 15:1) to afford Compound 6 (151.8 mg, 50.24%) as an orange solid.

LCMS: (ES, m/z): [M+H]⁺ 420

¹H NMR: (400 MHz, DMSO-d6, δ ppm): δ 1.74-1.76 (d, 3H), 3.48 (s, 3H), 6.09-6.12 (m, 1H), 6.27-6.30 (m, 1H), 7.09-7.10 (d, 1H), 7.27-7.29 (d, 1H), 7.35 (s, 1H), 7.46-7.50 (m, 1H), 7.73-7.75 (d, 1H), 7.78-7.80 (d, 1H), 7.87 (s, 1H), 8.56 (s, 1H).

### Example 7. Synthesis of Compound 7

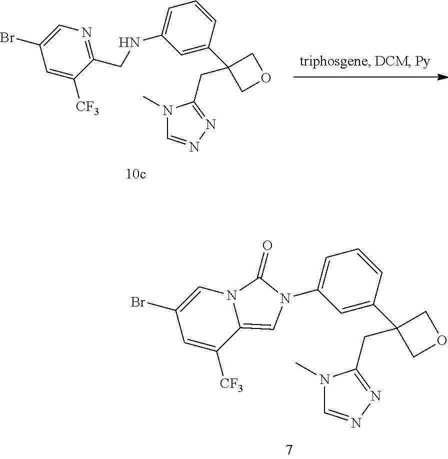 [SMILES: Cn1cnnc1CC1(c2cccc(NCc3ncc(Br)cc3C(F)(F)F)c2)COC1 ; Cn1cnnc1CC1(c2cccc(-n3cc4c(C(F)(F)F)cc(Br)cn4c3=O)c2)COC1]

### Synthesis of 7

To a stirred solution of 10c (4.40 g 9.123 mmol, 1.00 equiv) and pyridine (4.33 g, 54.741 mmol, 6.00 equiv) in DCM (250.00 mL) was added triphosgene (0.95 g, 3.193 mmol, 0.35 equiv) at 0° C. The resulting mixture was stirred 1 h at room temperature. The reaction was quenched by the addition of NaHCO₃ (aq.) (100 mL). The aqueous layer was extracted with CH₂Cl₂/MeOH=10/1 (3×200 mL). The resulting mixture was concentrated under vacuum. The residue was purified by trituration with methyl tert-butyl ether (50 mL). This resulted in Compound 7 (4.2 g, 90.57%) as a yellow solid.

LCMS: (ES, m/z): [M+H]⁺ 508

¹H NMR: (400 MHz, DMSO-d6, ppm): δ 2.97 (s, 3H), 3.53 (s, 2H), 4.91-4.96 (m, 4H), 6.89-6.91 (d, 1H), 7.18 (s, 1H), 7.39-7.45 (m, 3H), 7.73-7.75 (m, 1H), 8.04 (s, 1H), 8.20 (s, 1H).

### Example 8. Synthesis of Compound 8

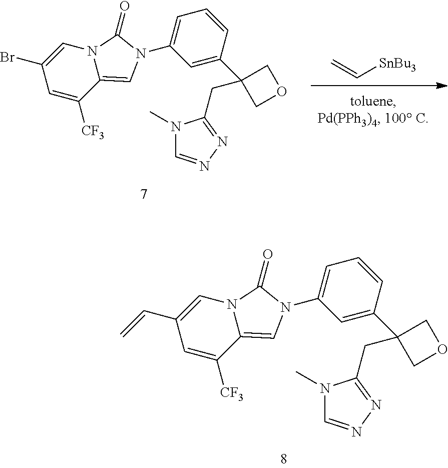

### Synthesis of 8

To a stirred solution of Compound 7 (680.00 mg, 1.338 mmol, 1.00 equiv) and tributyl(ethenyl)stannane (636.33 mg, 2.007 mmol, 1.50 equiv) in dioxane (10.00 mL) was added Pd(PPh₃)₄ (155.00 mg, 0.1338 mmol, 0.1 equiv) under nitrogen atmosphere. The resulting mixture was stirred overnight at 100° C. under nitrogen atmosphere. The resulting mixture was concentrated under reduced pressure. The residue was purified by silica gel column chromatography, eluted with CH₂Cl₂/MeOH (30:1) to afford Compound 8 (400 mg, crude). The crude product (20 mg) was purified by prep-HPLC with the following conditions (Column: XBridge Shied RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 45% B in 8 min, 45% B; Wave Length: 220 nm; RT1 (min): 7.92) to Compound 8 (7.4 mg) as a yellow solid.

LCMS: (ES, m/z): [M+H]⁺ 456

¹H NMR: (400 MHz, DMSO-d6, ppm): δ 2.98 (s, 3H), 3.54 (s, 2H), 4.92-4.97 (m, 4H), 5.25-5.27 (d, 1H), 5.80-5.85 (d, 1H), 6.66-6.73 (m, 1H), 6.88-6.90 (d, 1H), 7.39-7.43 (m, 4H), 7.75-7.77 (m, 1H), 7.91 (s, 1H), 8.21 (s, 1H).

### Example 9. Synthesis of Compound 9

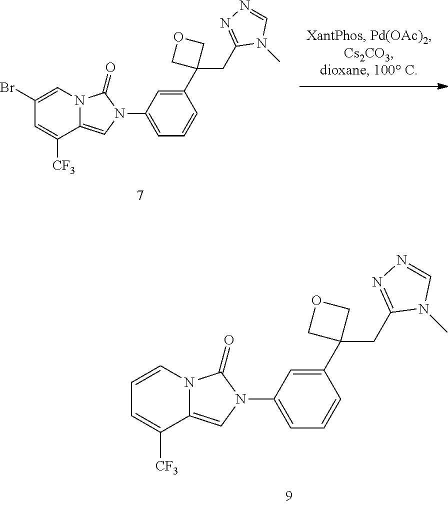 [SMILES: Cn1cnnc1CC1(c2cccc(-n3cc4c(C(F)(F)F)cc(Br)cn4c3=O)c2)COC1 ; Cn1cnnc1CC1(c2cccc(-n3cc4c(C(F)(F)F)cccn4c3=O)c2)COC1]

### Synthesis of 9

Into a 8-mL sealed tube purged and maintained with an inert atmosphere of nitrogen, was placed dioxane (2.00 mL), Compound 7 (50.00 mg, 0.098 mmol, 1.00 equiv), XantPhos (22.77 mg, 0.039 mmol, 0.40 equiv), Pd(OAc)₂ (4.42 mg, 0.020 mmol, 0.20 equiv), Cs₂CO₃ (96.15 mg, 0.295 mmol, 3.00 equiv). The resulting solution was stirred overnight at 100° C., The resulting mixture was diluted with water (50 mL). The aqueous layer was extracted with EtOAc (2×20 mL). The residue was purified by prep-TLC (CH₂Cl₂/MeOH 12:1) to afford crude product. The crude product (20 mg) was purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 45% B in 8 min, Wave Length: 220 am; RT1 (min): 7.35) to afford Compound 9 (2.5 mg, 5.91%) as a yellow solid.

LCMS: (ES, m/z): [M+H]⁺430

¹H NMR: (400 MHz, DMSO-d₆, ppm): δ 2.97 (s, 3H), 3.53 (s, 2H), 4.91-4.96 (m, 411), 6.27-6.30 (m, 1H), 6.88-6.90 (d, 1H), 7.09-7.11 (d, 1H), 7.38 (s, 1H), 7.40-7.42 (m, 2H), 7.74-7.79 (m, 2H), 8.20 (s, 1H).

### Example 10. Synthesis of Compound 10

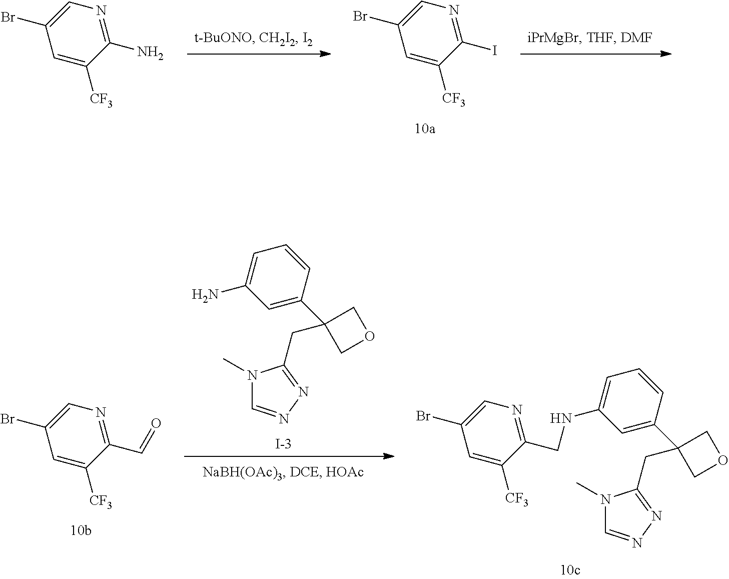

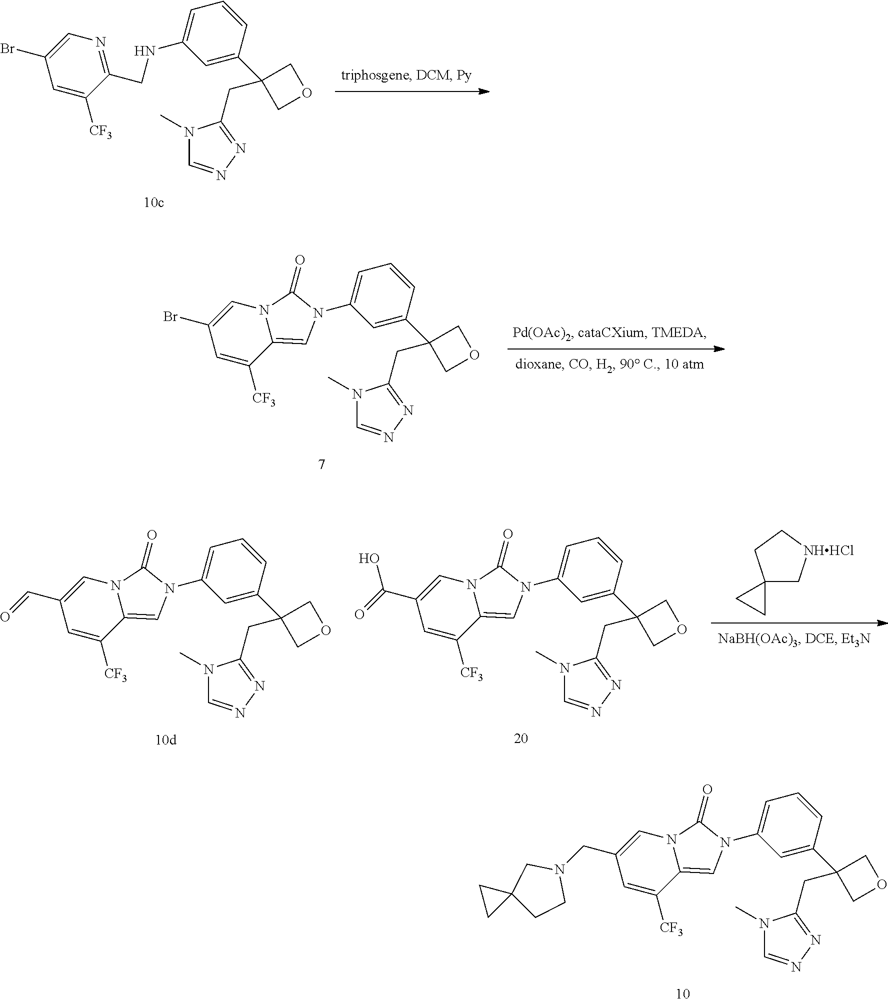

### Synthesis of 10a

To a stirred mixture of 5-bromo-3-(trifluoromethyl)pyridin-2-amine (25.00 g, 103.730 mmol, 1.00 equiv) in CH₂I₂ (75.00 mL) was added t-BuNO₂ (12.84 g, 124.515 mmol, 1.20 equiv) dropwise. The resulting mixture was stirred for 1 h at room temperature. To the above mixture was added I₂ (28.96 g, 114.102 mmol, 1.10 equiv) in portions. The resulting mixture was stirred for additional 6 h at room temperature. The reaction was quenched by the addition of NaHCO₃ (aq.) (300 mL). The aqueous layer was extracted with EtOAc (2×200 mL). The resulting mixture was concentrated under reduced pressure. The residue was purified by silica gel column chromatography, elated with PE/EtOAc (50:1) to afford 10a (17 g, 46.57%) as a light yellow oil.

### Synthesis of 10b

To a stirred solution of 10a (17.00 g, 48.310 mmol, 1.00 equiv) in THE (300.00 mL) was added i-PrMgBr (18.32 mL, 53.141 mmol, 1.10 equiv) dropwise at −78° C. under argon atmosphere. The resulting mixture was stirred for 30 min at −78° C. under argon atmosphere. To the above mixture was added DMF (7.06 g, 96.588 mmol, 2.00 equiv) dropwise at −78° C., The resulting mixture was stirred for additional 2 hours at −78° C. The reaction was quenched by the addition of NH₄Cl (aq.) (800 mL) at room temperature. The aqueous layer was extracted with EtOAc (2×400 mL). The resulting mixture was concentrated under reduced pressure. The residue was purified by silica gel column chromatography, eluted with PE/EtOAc (20:1) to afford 10b (4.5 g, 36.67%) as a white solid.

### Synthesis of 10c

To a stirred solution of 10b (3.50 g, 13,779 mmol, 1.00 equiv) and 1-3 (3.37 g, 13.795 mmol, 1.00 equiv) in DCE (50.00 mL) were added HOAc (1.65 g, 27.558 mmol, 2.00 equiv) and NaBH(OAc)₃ (5.84 g, 27.558 mmol, 2.00 equiv). The resulting mixture was stirred overnight at room temperature. The resulting mixture was diluted with water (100 mL). The aqueous layer was extracted with CH₂Cl₂ (3×150 mL). The resulting mixture was concentrated under vacuum. The residue was purified by silica gel column chromatography, eluted with CH₂Cl₂/MeOH (50:1) to afford 10c (4.5 g, 67.71%) as a light yellow solid.

### Synthesis of Compound 7

To a stirred solution of 10c (4.40 g, 9.123 mmol, 1.00 equiv) and pyridine (4.33 g, 54,741 mmol, 6.00 equiv) in DCM (250.00 mL) was added triphosgene (0.95 g, 3,193 mmol, 0.35 equiv) at 0° C. The resulting mixture was stirred 1 h at room temperature. The reaction was quenched by the addition of NaHCO₃ (aq.) (100 mL). The aqueous layer was extracted with CH₂Cl₂/MeOH=10/1 (3×200 mL). The resulting mixture was concentrated under vacuum. The residue was purified by trituration with methyl tert-butyl ether (50 mL). This resulted in Compound 7 (4.2 g, 90.57%) as a yellow solid.

### Synthesis of 10d

To a solution of Compound 7 (2.20 g, 4,328 mmol, 1.00 equiv), TMEDA (0.50 g, 4.328 mmol, 1.00 equiv) in dioxane (180.00 mL) was added butyldi-1-adamantylphosphine (0.31 g, 0.866 mmol, 0.20 equiv) and Pd(OAc)₂ (0.10 g, 0.433 mmol, 0.10 equiv) in an autoclave, After flushing the autoclave three times with CO/H₂ (1:1), the mixture was pressurized to 10 atm with CO/H₂ (1:1) at 90° C. and stirred overnight. The resulting mixture was concentrated under vacuum. The residue was purified by silica gel column chromatography, eluted with CH₂Cl₂/MeOH (20:1) to afford 10d (1.1 g, 55.56%) as a yellow solid, eluted with CH₂Cl₂/MeOH (5:1) to afford Compound 20 (600 mg) as a yellow solid.

### Synthesis of 10

To a stirred mixture of 10d (300.00 mg, 0.656 mmol, 1.00 equiv) and 5-azaspiro[2,4]heptane hydrochloride (175.27 mg, 1.312 mmol, 2.00 equiv) in DCE (10.00 mL) were added Et₃N (132.73 mg, 1.312 mmol, 2.00 equiv) and NaBH(OAc)₃ (278.01 Mg 1,312 mmol, 2.00 equiv). The resulting mixture was stirred overnight at room temperature. The resulting mixture was diluted with water (20 mL). The aqueous layer was extracted with CH₂Cl₂/MeOH=10/1 (3×30 mL). The resulting mixture was concentrated under vacuum. The crude product was purified by prep-HPLC with the following conditions (Column: XBridge Prep OBD C18 Column, 30*150 mm, 5 μm; Mobile Phase A: Water (10 mmol/L NH₄HCO₃+0.1% NH₃·H2O) Mobile Phase B: ACN; Flow rate: 60 mL/min; Gradient: 30% B to 50% B in 7 min, 50% B; Wave Length: 220 nm; RT1 (min): 6.57;) to afford Compound 10 (120.5 mg, 34.11%) as a yellow solid.

LCMS: (ES, m/z): [M+H]⁺: 539

¹H NMR: (400 MHz, DMSO-d6, ppm): δ 0.50-0.52 (m, 4H), 1.73-7.76 (t, 2H), 2.46 (s, 2H), 2.68-2.70 (t, 3H), 3.40 (s, 2H), 3.43 (2, 2H), 4.91-4.96 (m, 4H), 6.89-6.91 (d, 1H), 7.05 (s, 1H), 7.30 (s, 1H), 7.38-7.45 (m, 2H), 7.68 (s, 1H), 7.77-7.81 (d, 1H), 8.20 (s, 1H).

### Example 11. Synthesis of Compound 11

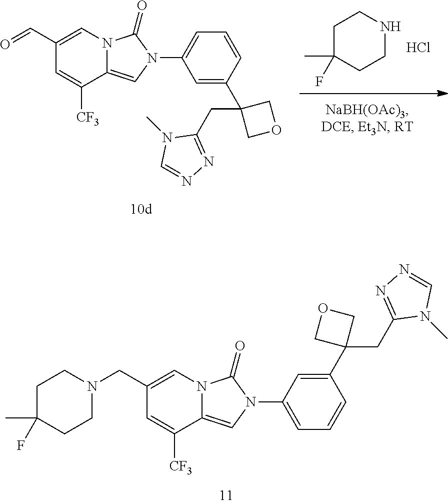

### Synthesis of 11

To a stirred solution/mixture of 10d (100.00 mg, 0.219 mmol, 1.00 equiv), 4-fluoro-4-methylpiperidine hydrochloride (67.17 mg, 0.438 mmol, 2.00 equiv) and Et₃N (44.24 mg, 0.438 mmol, 2.00 equiv) in DCE (2.00 mL) was added NaBH(OAc)₃ (92.67 mg, 0.438 mmol, 2.00 equiv). The resulting mixture was stirred overnight at room temperature. The reaction was quenched by the addition of water (10 mL). The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 50% B in 8 min, 50% B; Wave Length: 220 nm; RT1 (min): 7.83;) to afford Compound 11 (40.9 mg, 33.49%) as a yellow solid.

LCMS: (ES, m/z): [M+H]⁺ 559

¹H NMR: (400 MHz, DMSO-d6, ppm): δ 1.29-1.35 (d, 3H), 1.61-1.69 (m, 1H), 1.70-1.78 (m, 3H), 2.22-2.31 (m, 2H), 2.60-2.69 (m, 2H), 2.98 (s, 3H), 4.93-4.95 (m, 4H), 6.86-6.91 (d, 1H), 7.03 (s, 1H), 7.30 (s, 1H), 7.38-7.42 (m, 2H), 7.68 (s, 1H), 7.77-7.81 (d, 1H), 8.20 (s, 1H).

Alternatively, Compound 11 may be also prepared in the manner outlined below:

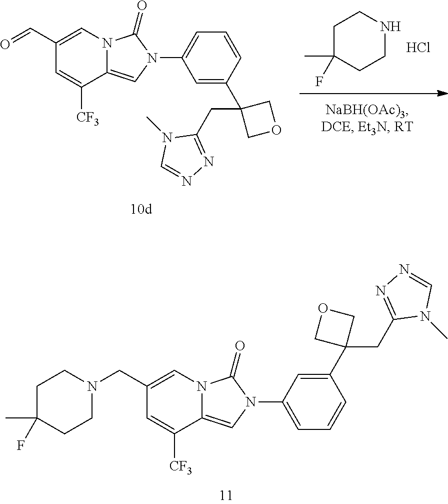

To a stirred mixture of 10d (3 g, 6.559 mmol, 1 equiv) and 4-fluoro-4-methylpiperidine hydrochloride (3.02 g, 19,677 mmol, 3 equiv) in DCE (50 mL) was added TEA (2.65 g, 26.236 mmol, 4 equiv). The resulting mixture was stirred for 2 h at room temperature. To the above mixture was added NaBH(OAc)3 (2.78 g, 13.118 mmol, 2 equiv), The resulting mixture was stirred for additional overnight at room temperature. The resulting mixture was diluted with water (20 mL). The resulting mixture was extracted with CH₂Cl₂ (3×25 mL). The combined organic layers were concentrated under reduced pressure. The residue was purified by reverse flash chromatography with the following conditions: column, C18 silica gel; mobile phase, MeCN in water (10 mmol NH4HCO3), 5% to 85% gradient in 40 min; detector, UV 254 van. This resulted in Compound 11 (9293 mg, 25.37%) as a yellow solid.

LC-MS-11: (ES, m/z): [M+H]⁺ 559. H-NMR-11: (400 MHz, DMSO-d6, ppm): δ 1.29-1.35 (d, 3H), δ 1.61-3 (m, 4H), δ 2.23-2.33 (m, 2H), δ 2.60-2.63 (m, 2H), δ 2.97 (s, 3H), δ 3.31 (s, 2H), δ 3.53 (s, 2H), δ 4.91-4.96 (m, 4H), δ 6.88-6.90 (d, 1H), δ 7.02 (s, 1H), δ 7.33 (s, 1H), S 7.38-7.42 (m, 2H), δ 7.69 (s, 1H), δ 7.75-7.77 (m, 1H), δ 8.21 (s, 1H).

### Example 12. Synthesis of Compound 12

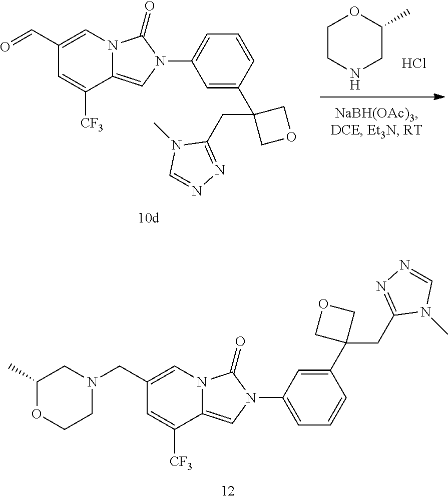

### Synthesis of 12

To a stirred mixture of 10d (100.00 mg, 1.00 equiv), (2R)-2-methylmorpholine hydrochloride (60.17 mg, 0.437 mmol, 2.00 equiv) and Et₃N (44.24 mg, 0.437 mmol, 2.00 equiv) in DCE (2.00 mL) was added NaBH(OAc)₃ (92.67 mg, 0.437 mmol, 2.00 equiv). The resulting mixture was stirred overnight at room temperature. The reaction was quenched by the addition of water (10 mL) at room temperature. The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep C18 OBD Column, 30*50 mm, 5 m 13 nm; Mobile Phase A: Water (20 mmol/L NH₄HCO₃), Mobile Phase B: ACN; Flow rate: 60 mL/min; Gradient: 18% B to 44% B in 8 min, 44% B; Wave Length: 220 nm; RT1 (min): 7.83;) to afford Compound 12 (30.8 mg) as a yellow solid.

LCMS: (ES, m/z): [M+H]⁺ 543. ¹H NMR: (400 MHz, DMSO-d6, ppm): δ 1.04-1.06 (d, 3H), 1.71-1.78 (m, 1H), 2.02-2.10 (m, 1H), 2.67-2.71 (m, 1H), 2.72-2.76 (m, 1H), 2.98 (s, 3H), 3.28 (s, 3H), 3.35-3.54 (m, 4H), 3.73-3.78 (d, 1H), 4.92-4.97 (m, 4H), 6.86-6.91 (d, 1H), 7.03 (s, 1H), 7.33 (s, 1H), 7.38-7.42 (m, 2H), 7.70 (s, 1H), 7.77-7.81 (d, 1H), 8.21 (s, 1H).

Alternatively, Compound 12 may also be prepared in the manner below:

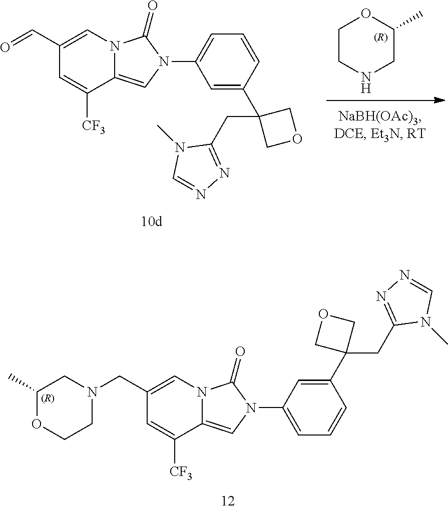

### 1. Synthesis of 12

To a stirred solution of 10d (2 g, 4.372 mmol, 1.00 equiv) and (2R)-2-methylmorpholine (1.33 g, 13.116 mmol, 3 equiv) in DCE (60 mL) was added NaBH(OAc)3 (1.85 g, 8.744 mmol, 2 equiv). The resulting mixture was stirred for 6 h at room temperature. The resulting mixture was diluted with water (50 mL). The resulting mixture was extracted with CH2Cl2/MeOH (2×50 mL). The combined organic layers were concentrated under reduced pressure. The residue was purified by reverse flash chromatography with the following conditions: column, C18 silica gel; mobile phase, MeOH in water, 10% to 65% gradient in 30 min; detector, UV 254 nm. This resulted in 12 (1.1056 g, 46.60%) as a yellow solid.

LC-MS-12: (ES, m/z): [M+H]⁺ 543. H-NMR-12: (400 MHz, DMSO-d6, ppm): δ 1.04-1.06 (d, 3H), δ 1.71-1.78 (m, 1H), δ 2.05-2.08 (m, 1H), δ 2.67-2.72 (m, 1H), δ 2.72-2.75 (m, 1H), δ 2.97 (s, 3H), δ 3.28 (s, 2H), δ 3.45-3.54 (m, 4H), δ 3.73-3.76 (d, 1H), δ 4.91-4.96 (m, 4H), S 6.88-6.91 (d, 1H), δ 7.02 (s, 1H), δ 7.33 (s, 1H), δ 7.40-7.42 (m, 2H), δ 7.68 (s, 1H), δ 7.77-7.81 (d, 1H), δ 8.21 (s, 1H).

### Example 13. Synthesis of Compound 13

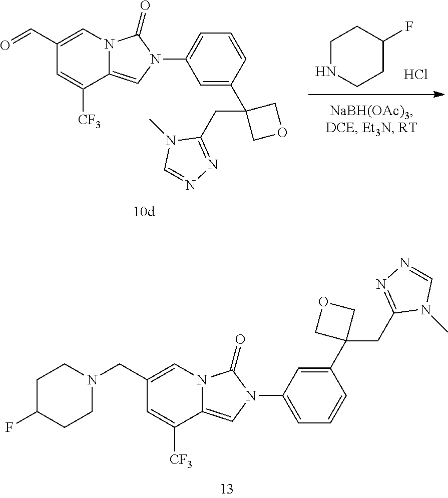 [SMILES: Cn1cnnc1CC1(c2cccc(-n3cc4c(C(F)(F)F)cc(C=O)cn4c3=O)c2)COC1 ; FC1CCNCC1 ; Cl ; Cn1cnnc1CC1(c2cccc(-n3cc4c(C(F)(F)F)cc(CN5CCC(F)CC5)cn4c3=O)c2)COC1]

### Synthesis of 13

To a stirred mixture of 10d (100.00 mg, 0.219 mmol, 1.00 equiv), 4-fluoropiperidine hydrochloride (61.04 mg, 0.437 mmol, 2.00 equiv) and Et₃N (44.24 mg, 0.438 mmol, 2.00 equiv) in DCE (2.00 mL) was added NaBH(OAc)₃ (92.67 mg, 0.438 mmol, 2.00 equiv). The resulting mixture was stirred overnight at room temperature. The reaction was quenched by the addition of water (10 mL). The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep OBD C18 Column, 30*150 mm, 5 m; Mobile Phase A: Water (20 mmol/L NH₄HCO₃), Mobile Phase B: ACN; Flow rate: 60 mL/min; Gradient: 22% B to 50% B in 8 min, 50% B; Wave Length: 220 nm; RT1 (min): 7.75) to afford Compound 13 (44.2 mg, 37.13%) as a yellow solid.

LCMS: (ES, m/z): [M+H]⁺ 545

¹H NMR: (400 MHz, DMSO-d6, ppm): δ 1.62-1.81 (m, 2H), 1.83-1.90 (m, 2H), 2.33-2.37 (m, 2H), 2.55-2.67 (m, 2H), 2.98 (s, 3H), 3.54 (s, 2H), 4.62-4.77 (m, 1H), 4.91-4.96 (m, 4H), 6.89-6.91 (d, 1H), 7.02 (s, 1H), 7.31 (s, 1H), 7.38-7.42 (m, 2H), 7.68 (s, 1H), 7.74-7.76 (d, 1H), 8.20 (s, 1H).

### Example 14. Synthesis of Compound 14

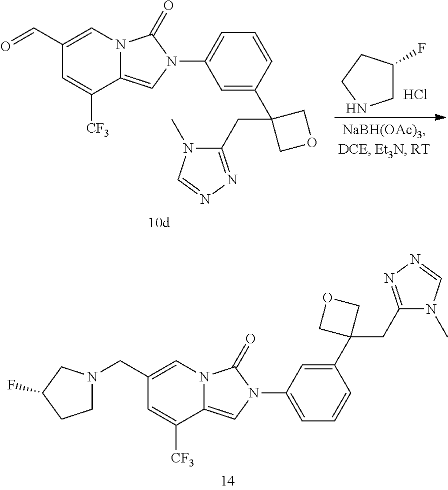 [SMILES: Cn1cnnc1CC1(c2cccc(-n3cc4c(C(F)(F)F)cc(C=O)cn4c3=O)c2)COC1 ; F[C@H]1CCNC1 ; Cl ; Cn1cnnc1CC1(c2cccc(-n3cc4c(C(F)(F)F)cc(CN5CC[C@H](F)C5)cn4c3=O)c2)COC1]

### Synthesis of 14

To a stirred mixture of 10d (100.00 mg, 0.219 mmol, 1.00 equiv), (3S)-3-fluoropyrrolidine hydrochloride (54.90 mg, 0.438 mmol, 2.00 equiv) and Et₃N (44.24 mg, 0.438 mmol, 2.00 equiv) in DCE (2.00 mL) was added NaBH(OAc)₃ (92.67 mg, 0.438 mmol, 2.00 equiv). The resulting mixture was stirred overnight at room temperature. The reaction was quenched by the addition of water (15 mL) at room temperature. The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 25% B to 45% B in 7 min, 45% B; Wave Length: 220 nm; RT1 (min): 6.32;) to afford Compound 14 (50.8 mg, 43.80%) as a yellow solid.

LCMS: (ES, m/z): [M+H]⁺ 531

¹H NMR: (400 MHz, DMSO-d6, ppm): δ 1.84-1.95 (m, 1H), 2.10-2.20 (m, 1H), 2.33-2.37 (m, 1H), 2.60-2.72 (m, 1H), 2.77-2.87 (m, 2H), 2.97 (s, 3H), 3.43 (s, 2H), 3.54 (s, 2H), 4.91-4.96 (m, 4H), 5.13-5.30 (m, 1H), 6.88-6.90 (d, 1H), 7.03 (s, 1H), 7.32 (s, 1H), 7.38-7.42 (m, 2H), 7.71 (s, 1H), 7.74-7.77 (m, 1H), 8.20 (s, 1H).

### Example 15. Synthesis of Compound 15

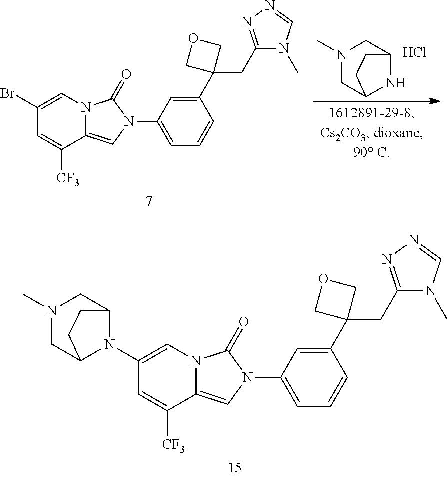

### Synthesis of 15

Into a 20 mL sealed tube purged and maintained with an inert atmosphere of nitrogen, was placed dioxane (10 mL), Compound 7 (200.00 mg, 0.393 mmol, 1.00 equiv), 3-methyl-3,8-diazabicyclo[3.2.1]octane hydrochloride (320.01 mg, 1.967 mmol, 5.00 equiv), Cs₂CO₃ (641.00 mg, 1.967 mmol, 5 equiv), Pd PEPPSI IPentCl (169.30 mg, 0.197 mmol, 0.50 equiv). The resulting solution was stirred overnight at 90° C. The resulting mixture was filtered, the filter cake was washed with CH₂Cl₂ (1×10 mL). The filtrate was concentrated under reduced pressure. The residue was purified by prep-TLC (CH₂Cl₂/MeOH 12:1) to afford the crude product. The residue was purified by reverse flash chromatography with the following conditions: column, C18 silica gel; mobile phase, A: Water B: MeCN, 45% B to 60% B gradient in 15 min; detector, UV 254 am. This resulted in Compound 15 (33.7 mg, 15.19%) as a yellow solid.

LCMS: (ES, m/z): [M+H]⁺ 554

¹H NMR: (400 MHz, DMSO-d₆, δ ppm): δ 1.84-1.86 (m, 4H), 2.12 (s, 3H), 2.77-2.34 (m, 2H), 2.48-2.50 (m, 2H), 2.98 (s, 3H), 3.53 (s, 2H), 4.09 (s, 2H), 4.90-4.96 (m, 4H), 6.86-6.88 (d, 1H), 6.93 (s, 1H), 7.14-7.16 (m, 1H), 7.28 (s, 1H), 7.37-7.41 (m, 2H), 7.74-7.76 (m, 1H), 8.20 (s, 1H).

### Example 16. Synthesis of Compound 16

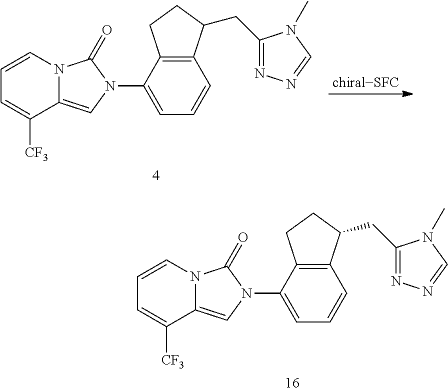 [SMILES: Cn1cnnc1CC1CCc2c1cccc2-n1cc2c(C(F)(F)F)cccn2c1=O ; Cn1cnnc1C[C@H]1CCc2c1cccc2-n1cc2c(C(F)(F)F)cccn2c1=O]

### Synthesis of 16

The Compound 4 (59.00 mg) was separated by prep-CHIRAL-SFC with the following conditions (Column: CHIRALPAK IG, 2*25 cm, 5 n; Mobile Phase A: Hex (0.2% TEA), Mobile Phase B: EtOH:DCM=1:1; Plow rate: 20 mL/min; Gradient: 40% B to 40% B in 15 min; Wave Length: 220/254 nm; RT1 (min): 10.54; RT2 (min): 12.60; the first peak was the product). This resulted in Compound 16 (15.9 mg) as a yellow solid.

LCMS: (ES, m/z): [M+H]⁺ 414

¹H NMR: (400 MHz, DMSO-d6 δ ppm) δ 1.76-1.81 (m, 1H), 2.25-2.30 (m, 1H), 2.78-2.92 (m, 3H), 3.20-3.34 (m, 1H), 3.69 (s, 3H), 3.71-3.73 (m, 1H), 6.27-6.31 (m, 1H), 7.07-7.08 (d, 1H), 7.09 (s, 1H), 7.30-7.32 (m, 3H), 7.79-7.81 (d, 1H), 8.38 (s, 1H).

### Example 17. Synthesis of Compound 17

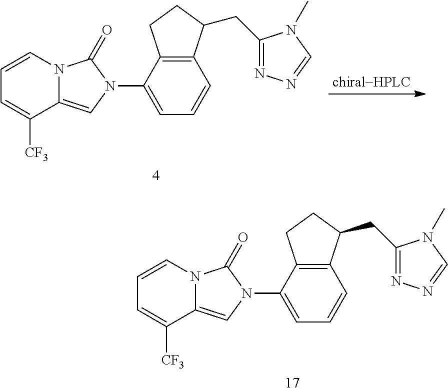 [SMILES: Cn1cnnc1CC1CCc2c1cccc2-n1cc2c(C(F)(F)F)cccn2c1=O ; Cn1cnnc1C[C@@H]1CCc2c1cccc2-n1cc2c(C(F)(F)F)cccn2c1=O]

### Synthesis of 17

Compound 4 (59.00 mg) was separated by prep-CHIRAL-HPLC with the following conditions (Column: CHIRALPAK IG, 2*25 cm, 5 μm; Mobile Phase A: Hex (0.2% TEA), Mobile Phase B: EtOH:DCM=1:1; Flow rate: 20 mL/min; Gradient: 40% B to 40% B in 15 min; Wave Length: 220/254 nm; RT2 (min): 12.60). This resulted in Compound 17 (17.7 mg) as a yellow solid.

LCMS: (ES, m/z): [M+H]⁺ 414

¹H NMR: (400 MHz, DMSO-d6, ppm): δ 1.78-1.82 (m, 1H), 2.26-2.34 (m, 1H), 2.80-2.89 (m, 3H), 3.20-3.25 (m, 1H), 3.69 (s, 3H), 3.71-3.73 (m, 1H), 6.28-6.31 (m, 1H), 7.08-7.10 (d, 1H), 7.14 (s, 1H), 7.29-7.35 (m, 3H), 7.79-7.81 (d, 1H), 8.39 (s, 1H).

### Example 18. Synthesis of Compound 18

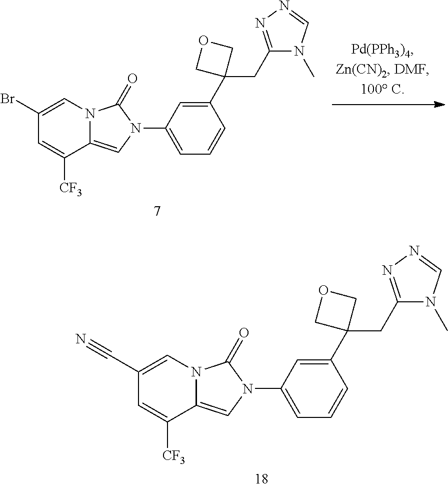 [SMILES: Cn1cnnc1CC1(c2cccc(-n3cc4c(C(F)(F)F)cc(Br)cn4c3=O)c2)COC1 ; Cn1cnnc1CC1(c2cccc(-n3cc4c(C(F)(F)F)cc(C#N)cn4c3=O)c2)COC1]

### Synthesis of 18

Into a 8-ml sealed tube purged and maintained with an inert atmosphere of nitrogen, was placed DMF (2.00 mL), Compound 7 (50.00 mg, 0.098 mmol, 1.00 equiv), Pd(PPh₃)4 (11.37 ag, 0.010 mmol, 0.10 equiv), Zn(CN)₂ (23.11 mg, 0.197 mmol, 2 equiv). The resulting solution was stirred for 3 hr at 100° C. The residue was purified by reverse flash chromatography with the following conditions: column, C18 silica gel; mobile phase, MeCN in water, 0% to 50% gradient in 20 min; detector, UV 254 nm. This resulted in 17.9 mg (39.24%) of Compound 18 as a dark yellow solid.

LCMS: (ES, m/z): [M+H]⁺ 455

¹H NMR: (400 MHz, DMSO-d₆, ppm): δ 2.98 (s, 3H), 3.53 (s, 2H), 4.89-4.96 (m, 4H), 6.91-6.93 (d, 1H), 7.30 (s, 1H), 7.42-7.43 (m, 2H), 7.52 (s, 1H), 7.71-7.74 (d, 1H), 8.20 (s, 1H), 8.71 (s, 1H).

### Example 19. Synthesis of Compound 19

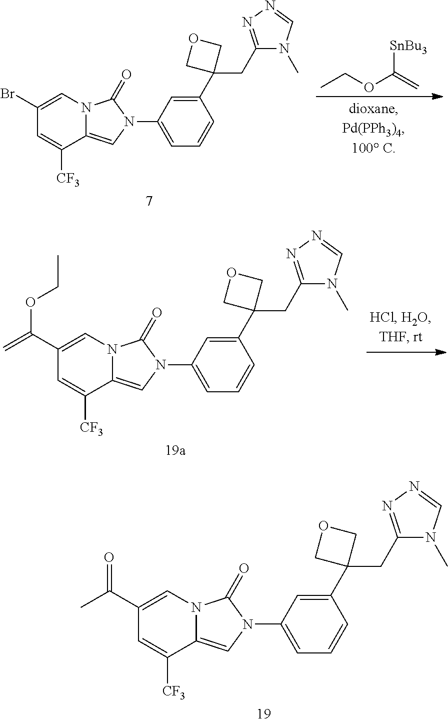

To a stirred mixture of Compound 7 (2000.00 mg, 3,935 mmol, 1.00 equiv) and tributyl(1-ethoxyethenyl)stannane (2131.56 tug, 5.902 mmol, 1.5 equiv) in dioxane (20.00 mL) was added Pd(PPh₃)₄ (454.68 mg, 0.393 mmol, 0.1 equiv) at room temperature under air atmosphere. The resulting mixture was stirred for 4 h at 100° C. under nitrogen atmosphere. The reaction was quenched by the addition of NH₄Cl (aq.) (60 mL) at room temperature. The aqueous layer was extracted with EtOAc (3×40 mL)e The resulting mixture was concentrated under reduced pressure. The residue was purified by silica gel column chromatography, eluted with CH₂Cl₂/MeOH (20:1) to afford 19a (1550 mg, 70.98%) as a yellow solid,

### Synthesis of 19

To a stirred mixture of 6-(1-ethoxyethenyl)-2-(3-[3-[(4-methyl-1,2,4-triazol-3-yl)methyl]oxetan-3-yl]phenyl)-8-(trifluoromethyl)imidazo[1,5-a]pyridin-3-one (19a) (1550.00 mg, 3,103 mmol, 1.00 equiv) in THF (10.00 mL) was added HCl (1.00 mL, 1M) and H₂O (10.00 mL) at room temperature. The resulting mixture was stirred overnight at room temperature. The resulting mixture was diluted with water (20 mL). The aqueous layer was extracted with CH₂Cl₂ (3×30 mL). The resulting mixture was concentrated under reduced pressure to afford crude product (1.44 g) as a yellow solid. The crude product (20 mg) was purified by prep-HPLC with the following conditions (Column: XBridge Prep OBD C18 Column, 30*150 mm, 5 μm; Mobile Phase A: Water (20 mmol/L NH₄HCO₃), Mobile Phase B: ACN; Flow rate: 60 mL/min; Gradient: 18% B to 45% B in 8 min, 45% B; Wave Length: 220 nm; RT1 (min): 7.48) to afford Compound 19 (16.8 mg) as a yellow solid.

LCMS: (ES, m/z): [M+H]⁺ 472

¹H NMR: (400 MHz, DMSO, ppm): δ 2.57 (s, 3H), 3.00 (s, 3H), 3.55 (s, 2H), 4.93-4.98 (m, 4H), 6.93-6.95 (d, 1H), 7.31 (s, 1H), 7.42-7.46 (m, 2H), 7.51 (s, 1H), 7.76-7.77 (m, 1H), 8.22 (s, 1H), 8.58 (s, 1H).

### Example 20. Synthesis of Compound 20

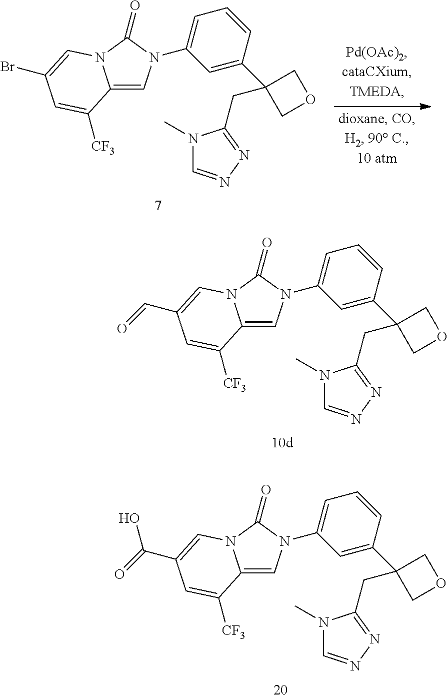 [SMILES: Cn1cnnc1CC1(c2cccc(-n3cc4c(C(F)(F)F)cc(Br)cn4c3=O)c2)COC1 ; Cn1cnnc1CC1(c2cccc(-n3cc4c(C(F)(F)F)cc(C=O)cn4c3=O)c2)COC1 ; Cn1cnnc1CC1(c2cccc(-n3cc4c(C(F)(F)F)cc(C(=O)O)cn4c3=O)c2)COC1]

### Synthesis of 20

To a solution of Compound 7 (2.20 g, 4.328 mmol, 1.00 equiv), TMEDA (502.95 mg, 4.328 mmol, 1.00 equiv) in dioxane (100.00 mL) was added butyldi-1-adamantylphosphine (0.31 g, 0.866 mmol, 0.20 equiv) and Pd(OAc)₂ (97.17 mg, 0.433 mmol, 0.10 equiv) in an autoclave. After flushing the autoclave three times with CO/H₂ (1:1), the mixture was pressurized to 10 atm with CO/H₂ (1:1) at 100° C. and stirred overnight. The residue was purified by silica gel column chromatography, eluted with CH₂CO₂/MeOH (20:1) to afford 10d (1.1 g, 55.56%) as a yellow solid, eluted with CH₂Cl₂/MeOH (5:1) to afford Compound 20 (600 mg) as a yellow solid. The crude product (50 mg) was purified by prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 9% B to 35% B in 8 min, 35% B; Wave Length: 220 nm; RT1 (min): 6.08;) to afford Compound 20 (11.6 mg) as a yellow solid.

LCMS: (ES, m/z): [M+H]⁺ 474

¹H NMR: (400 MHz, DMSO-d6, ppm): δ 2.97 (s, 3H), 3.54 (s, 2H), 4.91-4.97 (m, 4H), 6.87-6.89 (d, 1H), 7.31 (s, 1H), 7.38-7.44 (m, 3H), 7.75-7.77 (d, 1H), 8.16 (s, 1H), 8.20 (s, 1H).

### Example 21. Synthesis of Compound 21

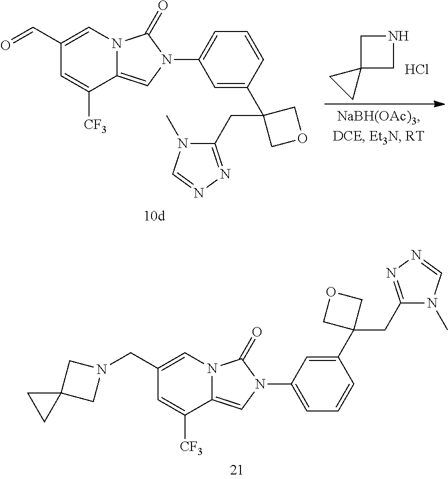 [SMILES: Cn1cnnc1CC1(c2cccc(-n3cc4c(C(F)(F)F)cc(C=O)cn4c3=O)c2)COC1 ; C1CC12CNC2 ; Cl ; Cn1cnnc1CC1(c2cccc(-n3cc4c(C(F)(F)F)cc(CN5CC6(CC6)C5)cn4c3=O)c2)COC1]

### Synthesis of 21

To a stirred mixture of 10d (100.00 mg, 0.219 mmol, 1.00 equiv), 5-azaspiro[2.3]hexane hydrochloride (52.29 mg, 0.438 mmol, 2.00 equiv) and Et₃N (44.24 mg, 0.438 mmol, 2.00 equiv) in DCE (2.00 mL) was added NaBH(OAc)₃ (92.67 mg, 0.438 mmol, 2.00 equiv). The resulting mixture was stirred overnight at room temperature. The reaction was quenched by the addition of water (10 mL). The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 25% B to 45% B in 7 min, 45% B; Wave Length: 220 nm; RT1 (min): 6.77) to afford Compound 21 (38.6 mg, 33.66%) as a yellow solid.

LCMS: (ES, m/z): [M+H]⁺ 525

¹H NMR: (400 MHz, DMSO-d6, ppm): δ 0.51 (s, 4H), 2.97 (s, 3H), 3.29 (s, 4H), 3.46 (s, 2H), 3.53 (s, 2H), 4.91-4.96 (m, 4H), 6.88-6.90 (d, 1H), 7.00 (s, 1H), 7.30 (s, 1H), 7.38-7.42 (m, 2H), 7.67 (s, 1H), 7.74-7.76 (m, 1H), 8.20 (s, 1H).

### Example 22. Synthesis of Compound 22

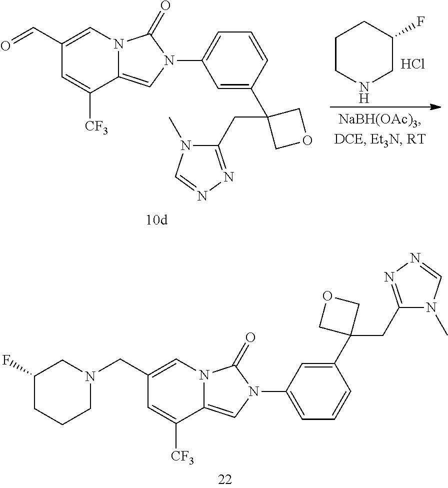 [SMILES: Cn1cnnc1CC1(c2cccc(-n3cc4c(C(F)(F)F)cc(C=O)cn4c3=O)c2)COC1 ; F[C@H]1CCCNC1 ; Cl ; Cn1cnnc1CC1(c2cccc(-n3cc4c(C(F)(F)F)cc(CN5CCC[C@H](F)C5)cn4c3=O)c2)COC1]

### Synthesis of 22

To a stirred mixture of 10d (100.00 mg, 0.219 mmol, 1.00 equiv), (3S)-3-fluoropiperidine hydrochloride (61.04 mg, 0.438 mmol, 2.00 equiv) and Et₃N (44.24 mg, 0.438 mmol, 2.00 equiv) in DCE (2.00 mL) was added NaBH(OAc)₃ (92.67 mg, 0.438 mmol, 2.00 equiv). The resulting mixture was stirred overnight at room temperature. The reaction was quenched by the addition of water (10 mL) at room temperature. The aqueous layer was extracted with CH₂Cl₂/MeOH=10/1 (3×15 mL). The resulting mixture was concentrated under reduced pressure. The crude product was purified by prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 25% B to 50% B in 8 min, 50% B; Wave Length: 220 nm; RT1 (min): 7.65;) to afford Compound 22 (42.9 mg, 36.03%) as a yellow solid.

LCMS: (ES, m/z): [M+H]⁺ 545

¹H NMR: (400 MHz, DMSO-d6, ppm): δ 1.44-1.67 (m, 2H), 1.72-1.82 (m, 2H), 2.28-2.39 (m, 1H), 2.42-2.50 (m, 2H), 2.71-2.75 (m, 1H), 2.98 (s, 3H), 3.54 (s, 2H), 4.58-4.72 (m, 1H), 4.91-4.96 (m, 4H), 6.89-6.91 (d, 1H), 7.01 (s, 1H), 7.31 (s, 1H), 7.38-7.42 (m, 2H), 7.69 (s, 1H), 7.74-7.75 (d, 1H), 8.20 (s, 1H).

### Example 23. Synthesis of Compound 23

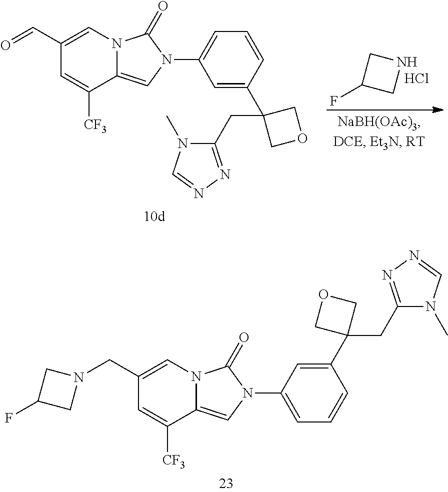

To a stirred mixture of 10d (1.00 equiv), 3-fluoroazetidine hydrochloride (2.00 equiv) and Et₃N (2 equiv) in DCE (2 mL) was added NaBH(OAc)₃ (2 equiv). The resulting mixture was stirred overnight at room temperature. The reaction was quenched by the addition of water (10 mL) at room temperature. The aqueous layer was extracted with CH₂Cl₂/MeOH=10/1 (3×15 mL). The resulting mixture was concentrated under reduced pressure. The crude product was purified by prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 30% B to 35% B in 7 min, 35% B; Wave Length: 254/220 nm; RT1 (min): 7.13;) to afford Compound 23 (26.5 mg) as a yellow solid.

LCMS: (ES, m/z): [M+H]⁺ 517

¹H NMR: (400 MHz, DMSO-d6, ppm): δ 2.97 (s, 3H), 3.13-3.17 (m, 1H), 3.19-3.23 (m, 1H), 3.44 (s, 2H), 3.50-3.60 (m, 4H), 4.91-4.97 (m, 4H), 5.11-5.30 (m, 1H), 6.88-6.91 (d, 1H), 6.98 (s, 1H), 7.30 (s, 1H), 7.38-7.42 (m, 2H), 7.70-7.76 (m, 2H), 8.20 (s, 1H).

### Example 24. Synthesis of Compound 24

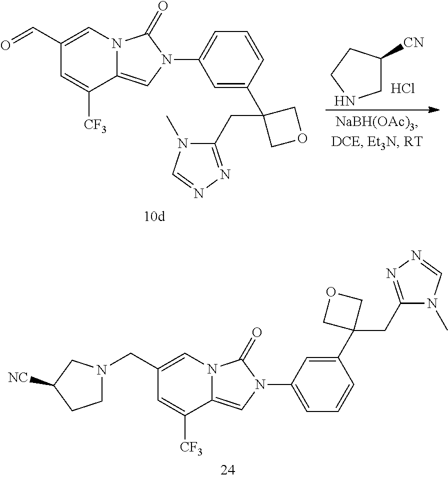

### Synthesis of 24

To a stirred mixture of 10d (100.00 mg, 0.219 mmol, 1.00 equiv), (3R)-pyrrolidine-3-carbonitrile hydrochloride (57.97 mg, 0.437 mmol, 2 equiv) and Et₃N (44.24 mg, 0.437 mmol, 2 equiv) in DCE (2.00 mL) was added NaBH(OAc)₃ (92.67 mg, 0.437 mmol, 2 equiv). The resulting mixture was stirred overnight at room temperature. The reaction was quenched by the addition of water (10 mL). The aqueous layer was extracted with CH₂Cl₂/MeOH=10/1 (3×15 mL). The resulting mixture was concentrated under vacuum. The crude product was purified by prep-HPLC with the following conditions (Column: YMC-Actus Triart C18 ExRS, 30*150 mm, 5 m; Mobile Phase A: Water (10 mmol/L NH₄HCO₃), Mobile Phase B: ACN; Flow rate: 60 mL/min; Gradient: 32% B to 35% B in 7 min, 35% B; Wave Length: 254/220 nm; RT1 (min): 7.0;) to afford Compound 24 (39.5 mg, 33.61%) as a yellow solid.

LCMS: (ES, m/z): [M+H]⁺ 538

¹H NMR: (400 MHz, DMSO-d6, ppm): δ 1.92-2.00 (m, 1H), 2.16-2.25 (m, 1H), 2.50-2.51 (m, 1H), 2.67-2.75 (m, 2H), 2.78-2.82 (m, 1H), 2.98 (s, 3H), 3.25-3.30 (m, 2H), 3.45 (s, 2H), 3.54 (s, 2H), 4.91-4.97 (m, 4H), 6.89-6.91 (d, 1H), 7.03 (s, 1H), 7.31 (s, 1H), 7.38-7.42 (m, 2H), 7.72-7.77 (m, 2H), 8.20 (s, 1H).

### Example 25. Synthesis of Compound 25

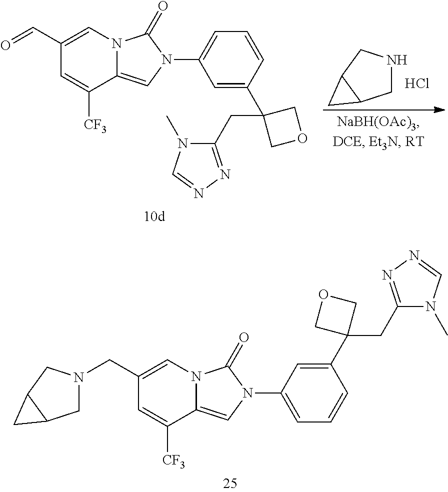

### Synthesis of 25

To a stirred mixture of 10d (100.00 mg, 0.219 mmol, 1.00 equiv) and 3-azabicyclo[3.1.0]hexane hydrochloride (52.29 mg, 2.00 equiv) in DCE (2.00 mL) were added Et₃N (44.24 mg, 0.438 mmol, 2.00 equiv) and NaBH(OAc)₃ (92.67 mg, 0.438 mmol, 2.00 equiv). The resulting mixture was stirred overnight at room temperature. The resulting mixture was diluted with water (10 mL). The aqueous layer was extracted with CH₂Cl₂/MeOH=10/1 (3×15 mL). The resulting mixture was concentrated under vacuum. The crude product was purified by prep-HPLC with the following conditions (Column: Kinetex EVO C18 Column, 30*150, 5 μm; Mobile Phase A: Water (10 mmol/L NH₄HCO₃+0.1% NH₃·H₂O), Mobile Phase B: ACN; Flow rate: 60 mL/min; Gradient: 30% B to 50% B in 7 min, 50% B; Wave Length: 220 nm; RT1 (min): 6.63;) to afford Compound 25 (39.2 mg, 34.18%) as a yellow solid.

LCMS: (ES, m/z): [M+H]⁺ 525

¹H NMR: (400 MHz, DMSO-d6, ppm): δ 0.36-0.37 (m, 1H), 0.61-0.64 (m, 1H), 1.38-1.39 (m, 2H), 2.34-2.36 (m, 2H), 2.87-2.89 (m, 2H), 2.97 (s, 3H), 3.39 (s, 2H), 3.53 (s, 2H), 4.91-4.96 (m, 4H), 6.88-6.90 (d, 1H), 6.95 (s, 1H), 7.30 (s, 1H), 7.38-7.42 (m, 2H), 7.64 (s, 1H), 7.74-7.76 (m, 1H), 8.20 (s, 1H).

### Example 26. Synthesis of Compound 26

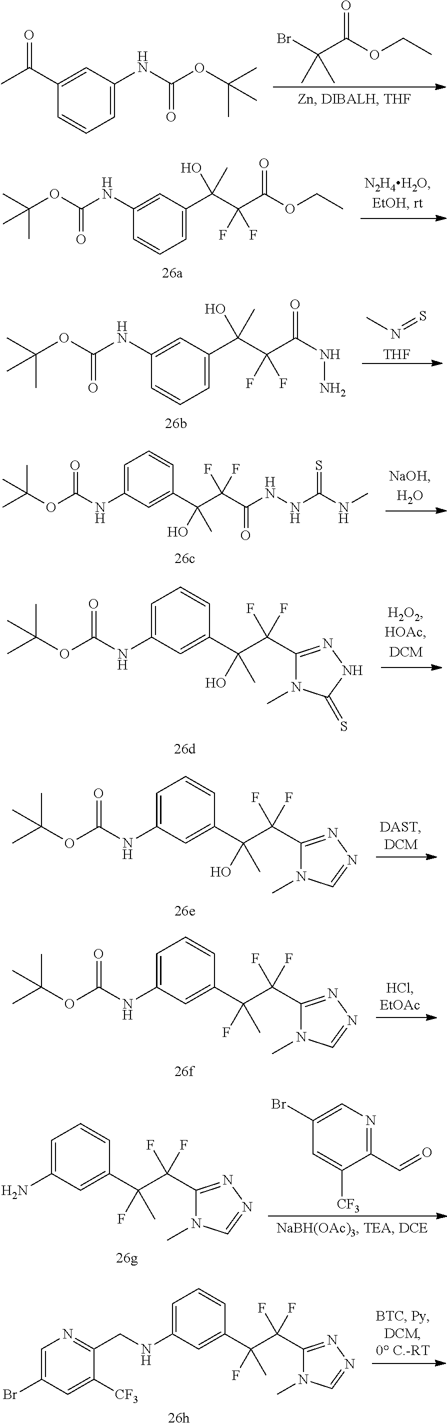

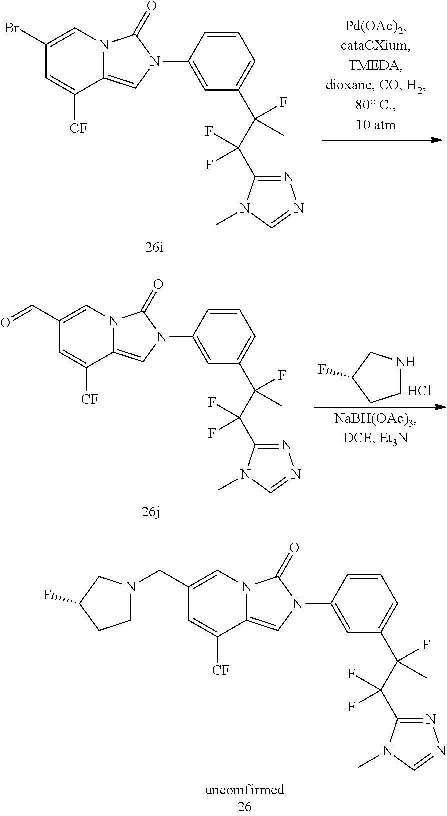

### Synthesis of 26a

To a mixture of Zn (23.77 g, 0.363 mmol, 4.5 equiv) and ethyl 2-bromo-2,2-difluoroacetate (0.2 g, 9.85 mmol) in THF (200 mL) was added DIBAL-H (0.46 g, 0.003 mmol, 0.04 equiv) at 30° C. Then the mixture was stirred for 1 h at 30° C. Then tert-butyl N-(3-acetylphenyl)carbamate (19.00 g, 80.754 mmol, 1.0 equiv) and ethyl 2-bromo-2,2-difluoroacetate (24.59 g, 0.121 mmol, 1.5 equiv) in THF (200 mL) was added dropwise at 40° C. and stirred for 3 h at 40° C. The mixture was filtered and the filtrate was poured into saturated NH₄Cl (200 mL). Then the mixture extracted with EtOAc (3×200 mL). The residue was purified by silica gel column chromatography, eluted with PE/EtOAc (50:1) to afford 26a (11.1 g 36.34%) as light yellow oil.

### Synthesis of 26b

To a solution of 26a (11.00 g, 30.609 mmol, 1.00 equiv) in EtOH (150 mL) was added hydrazine hydrate (98%) (7.66 g, 153.046 mmol, 5 equiv). Then the mixture was stirred at 25° C. for 16 h. The resulting mixture was diluted with water (200 mL). The aqueous layer was extracted with CH₂Cl₂ (4×100 mL). The resulting mixture was concentrated under vacuum. The crude product was used in the next step directly without further purification. This resulted in 26b (10.1 g, 92.49%) as a light yellow solid.

### Synthesis of 26c

To a solution of 26b (10.00 g, 28.956 mmol, 1.00 equiv) in THF (100 mL) was added methyl isothiocyanate (4.23 g, 0.058 mmol, 2 equiv). Then the mixture was stirred at 70° C. for 2 h. The mixture was concentrated to give the crude product 26c (12 g, 84.18%) as a yellow oil, which was used without purification.

### Synthesis of 26d

A solution of 26c (12 g, 28.677 mmol, 1.00 equiv) in NaOH (1.00 M, 100 mL) was stirred at 50° C. for 2 h. The mixture was acidified to pH 7 with HC (1 M) and filtered. This resulted in 26d (10 g, 79.25%) as an off-white solid.

### Synthesis of 26e

To a stirred solution of 26d (10.00 g, 24.973 mmol, 1.00 equiv) in DCM (100.00 l) was added H₂O₂ (8.49 g, 74,879 mmol, 3.00 equiv, 30%) in HOAc (3.00 g, 49.957 mmol, 2.00 equiv) at room temperature under air atmosphere. The resulting mixture was stirred for 3h at room temperature under air atmosphere. The mixture was basified to pH 8 with saturated NaHCO₃ (aq.) and quenched with Na₂SO₃ (aq.). The aqueous layer was extracted with CH₂Cl₂ (2×100 mL). The residue was purified by silica gel column chromatography, eluted with PE/EtOAc (1:1) to afford 26e (4.5 g, 46.96%) as an off-white solid.

### Synthesis of 26f

To a stirred solution of 26e (4.50 g, 12.215 mmol, 1.00 equiv) in DCM (100.00 mL) was added DAST (5.91 g, 0.037 mmol, 3.00 equiv) dropwise at room temperature under nitrogen atmosphere. The resulting mixture was stirred for 3h at room temperature. The reaction was quenched with NaHCO₃ (aq.) at room temperature. The aqueous layer was extracted with CH₂Cl₂ (2×100 mL). The residue was purified by silica gel column chromatography, eluted with hexane/EtOAc (1:1) to afford 26f (3.5 g, 72.72%) as an off-white solid.

### Synthesis of 26g

Into a 100-mL round-bottom flask purged and maintained with an inert atmosphere of nitrogen, was placed HCl (g) in EtOAc (50.00 mL, 875.874 mmol, 92.69 equiv). This was followed by the addition of 26g (3.50 g, 9.450 mmol, 1.00 equiv) at room temperature. The resulting solution was stirred overnight at room temperature. The resulting mixture was concentrated under reduced pressure. This resulted in 3 g (93.15%) of 26g as an off-white solid.

### Synthesis of 26h

Into a 100-mL round-bottom flask purged and maintained with an inert atmosphere of nitrogen, was placed DCE (20.00 mL), 26g (1.50 g, 4.890 mmol, 1.00 equiv), TEA (0.49 g, 0.005 mmol, 1 equiv), 5-bromo-3-(rifluoromethyl)pyridine-2-carbaldehyde (1.24 g, 0.005 mmol, 1.00 equiv), NaBH(OAc)₃ (3.11 g, 0.015 mmol, 3 equiv). The resulting solution was stirred overnight at room temperature. The resulting mixture was diluted with DCM (100 mL), washed with 50 ml of water. The residue was purified by prep-TLC (CH₂Cl₂/MeOH 12:1) to afford 26h (1.2 g, 45.86%) as a light yellow solid.

### Synthesis of 26i

To a stirred solution of 26h (1.20 g, 2.361 mmol, 1.00 equiv) and pyridine (1.12 g 0.014 mmol, 6 equiv) in DCM (60.00 mL) was added triphosgene (0.25 g, 0.001 mmol, 0.35 equiv) at 0° C. under nitrogen atmosphere. The resulting mixture was stirred for 10 min at room temperature. The resulting mixture was washed with 30 mL of NaHCO₃ (aq.). The residue was purified by prep-TLC (CH₂Cl₂/MeOH 12:1) to afford 26i (1.1 g, 82.39%) as a yellow solid.

### Synthesis of 26j

To a solution of 26i (1.00 g, 1.872 mmol, 1.00 equiv) in dioxane (30.00 mL) was added cataCXium (0.13 g, 0.363 mmol, 0.19 equiv), Pd(OAc)2 (0.04 g, 0.178 mmol, 0.10 equiv), TMEDA (0.44 g, 3.744 mmol, 2.00 equiv) in a pressure tank. The mixture was purged with nitrogen for 3 min and then was pressurized to 10 atm with CO/H₂ (1:1) at 80° C. and stirred overnight. The resulting mixture was concentrated under reduced pressure. The resulting mixture was diluted with water (100 mL). The aqueous layer was extracted with CH₂Cl₂ (2×100 mL). The residue was purified by prep-TLC (CH₂Cl₂/MeOH 20:1) to afford 26j (500 mg, 50.29%) as a yellow solid,

### Synthesis of 26

To a stirred solution of 26j (250.00 mg, 0.517 mmol, 1.00 equiv) and (3S)-3-fluoropyrrolidine hydrochloride (194.83 mg, 1.552 mmol, 3.00 equiv) in DCE (10.00 mL) was added Et₃N (157.01 mg, 1.552 mmol, 3 equiv) at room temperature under nitrogen atmosphere. The resulting mixture was stirred for 10 min at room temperature. To the above mixture was added NaBH(OAc)3 (328.85 mg, 1.552 mmol, 3 equiv), and then the mixture was stirred overnight at room temperature. The resulting mixture was washed with 10 mL of water. The residue was purified by prep-TLC (CH₂Cl₂/MeOH 20:1) to afford the crude product (up). The crude product (120 mg) was purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 57% B in 8 min, 57% B; Wave Length: 220 nm; RT (min): 7.23) to afford Compound 26 (59.3 mg, 20.42%) as a yellow solid.

LCMS: (ES, m/z): [M+H]⁺ 557

¹H NMR: (400 MHz, DMSO-d6, ppm): δ 1.83-1.95 (m, 1H), 1.99-2.17 (m, 3H), 2.19-2.24 (m, 1H), 2.41-2.49 (m, 1H), 2.67-2.71 (m, 1H), 2.75-2.84 (m, 2H), 3.31-3.52 (m, 5H), 5.16-5.29 (m, 1H), 7.05 (s, 1H), 7.36-7.38 (m, 2H), 7.56-7.60 (m, 1H), 7.73-7.75 (d, 1H), 7.89-7.92 (m, 2H), 8.64 (s, 1H).

### Example 27. Synthesis of Compound 27

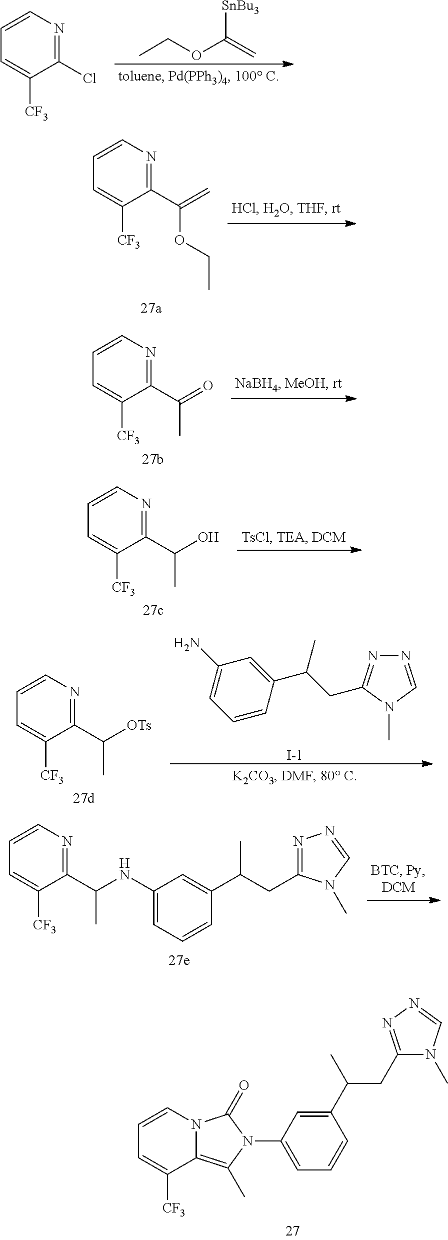

### Synthesis of 27a

To a stirred solution of 2-chloro-3-(trifluoromethyl) pyridine (1.00 g, 5.508 mmol, 1.00 equiv) in toluene (10.00 mL) was added tributyl(1-ethoxyethenyl) stannane (2.98 g, 8,263 mmol, 1.5 equiv) and Pd(PPh₃)4 (0.64 g, 0.551 mmol, 0.1 equiv) at room temperature under nitrogen atmosphere. The resulting mixture was stirred for overnight at 100° C. under nitrogen atmosphere. The residue was washed with water (15 mL). The resulting mixture was concentrated under vacuum. The residue was purified by silica gel column chromatography, eluted with PE/EtOAc (40:1) to afford 27a (1.1 g, 86.35%) as a white oil.

### Synthesis of 27b

To a stirred solution of 27a (1.08 g, 4.973 mmol, 1.00 equiv) in THF (2.00 mL) was added HCl (2.00 mL, 1M) dropwise at room temperature under nitrogen atmosphere. The resulting mixture was stirred for 2h at room temperature under nitrogen atmosphere. The mixture was quenched with saturated Na₂CO₃ (aq.)(20 mL). The resulting mixture was extracted with EtOAc (3×25 mL), dried over anhydrous Na₂SO₄. The resulting mixture was concentrated under vacuum to afford 27b (850 mg, 90.42%) as a yellow oil.

### Synthesis of 27c

To a stirred solution of 27b (800.00 mg, 4.230 mmol, 1.00 equiv) in MeOH (10.00 mL) was added NaBH₄ (80.01 mg, 2,115 mmol, 0.5 equiv) at room temperature under nitrogen atmosphere. The resulting mixture was stirred for 1h at room temperature under nitrogen atmosphere. The resulting mixture was diluted with 40 mL of water. The resulting mixture was extracted with 3×40 mL of EtOAc. The residue was purified by prep-TLC (CH₂Cl₂/MeOH 20:1) to afford 27c (400 mg, 44.53%) as a yellow oil.

### Synthesis of 27d

To a stirred solution of 27c (500.00 mg, 2.616 mmol, 1.00 equiv) in DCM (6 mL) was added TsCl (997.35 mg, 5.232 mmol, 2.00 equiv) and TEA (794.05 mg, 7.848 mmol, 3.00 equiv) dropwise at 0° C. under nitrogen atmosphere. The resulting mixture was stirred for 2h at 0° C. under nitrogen atmosphere. The resulting mixture was washed with 2×5 mL of water. The organic layer was concentrated under vacuum. The residue was purified by prep-TLC (PE/EtOAc 2:1) to afford 27d (500 mg, 52.03%) as a yellow oil.

### Synthesis of 27e

To a stirred solution of 27d (500.00 mg, 1.448 mmol, 1.00 equiv) in DMF (6.00 mL) was added 3-[1-(4-methyl-1,2,4-triazol-3-yl) propan-2-yl] aniline (375.78 mg, 1.738 mmol, 0.20 equiv) and K₂CO₃ (60030 mg, 4.344 mmol, 3.00 equiv) at room temperature under nitrogen atmosphere. The resulting mixture was stirred for 3h at 80° C. under nitrogen atmosphere. The resulting mixture was diluted with 20 mL of water. The resulting mixture was extracted with 3×20 mL of EtOAc. The residue was purified by prep-TLC (CH₂Cl₂/MeOH 20:1) to afford 27e (80 mg, 13.20%) as a yellow oil.

### Synthesis of 27

To a stirred solution of 27e (80.00 mg, 0.205 mmol, 1.00 equiv) in DCM (2 mL) was added pyridine (9872 mg, 1.230 mmol, 6.00 equiv) and BTC (16.38 mg, 0,072 mmol, 0.35 equiv) at 0° C. under nitrogen atmosphere. The resulting mixture was stirred for 10 min at 0° C. under nitrogen atmosphere. The resulting mixture was diluted with 15 mL of water, extracted with DCM (3×20 mL). The crude product was purified by reverse flash chromatography with the following conditions: column, C18; mobile phase, A: water (0.1% NH₄CO₃), B: CH₃CN, 45% B to 55% B gradient in 10 min; detector, UV 254 nm. The resulting mixture was concentrated under reduced pressure to afford Compound 27 (15.6 mg 17.95%) as a yellow solid.

LCMS: (ES, m/z): [M+H]⁺ 416

¹H NMR: (400 MHz, DMSO-d6, ppm): δ 1.31-1.33 (d, 3H), 1.99 (s, 3H), 2.99-3.03 (m, 2H), 3.30-3.33 (m, 1H), 3.42 (s, 3H), 6.15-6.19 (m, 1H), 7.01-7.02 (d, 1H), 7.31-7.38 (m, 2H), 7.41-7.42 (d, 1H), 7.46-7.49 (m, 1H), 7.70-7.72 (d, 1H), 8.27 (s, 1H).

### Example 28. Synthesis of Compound 28

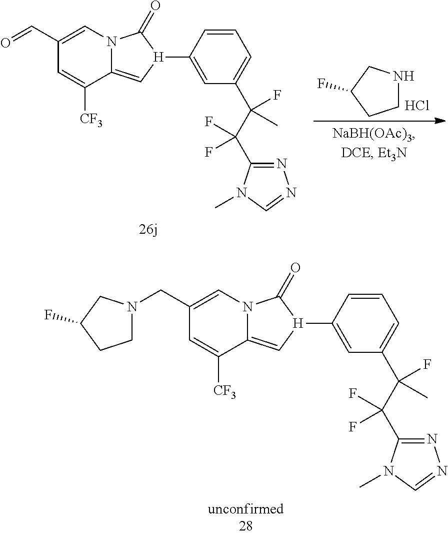

### Synthesis of 28

To a stirred solution of 26j (250.00 mg, 0.517 mmol, 1.00 equiv) and (3S)-3-fluoropyrrolidine hydrochloride (194.83 mg, 1,552 mmol, 3.00 equiv) in DCE (10.00 mL) was added Et₃N (157.01 mg, 1,552 mmol, 3 equiv) at room temperature under nitrogen atmosphere. The resulting mixture was stirred for 10 min at room temperature. To the above mixture was added NaBH(OAc)₃ (328.85 mg, 1.552 mmol, 3 equiv), and then the mixture was stirred overnight at room temperature. The resulting mixture was washed with 10 ml, of water. The residue was purified by prep-TLC (CH₂Cl₂/MeOH 20:1) to afford the crude product (down). The crude product (100 mg) was purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 60% B in 8 min, 60% B; Wave Length: 220 urn; RT1 (min): 7.55;) to afford Compound 28 (27.2 ng, 9.33%) as a yellow solid.

LCMS: (ES, m/z): [M+H]⁺ 557

¹H NMR: (400 MHz, DMSO-d6, ppm): δ 1.83-1.95 (m, 1H), 1.99-2.17 (m, 3H), 2.19-2.24 (m, 1H), 2.41-2.49 (m, 1H), 2.67-2.91 (m, 3H), 3.31-3.36 (m, 1H), 3.36-3.42 (m, 1H), 3.52 (s, 3H), 5.17-5.31 (m, 1H), 7.06 (s, 1H), 7.37-7.39 (m, 2H), 7.56-7.60 (m, 1H), 7.73-7.75 (d, 1H), 7.89-7.92 (m, 2H), 8.64 (s, 1H).

### Example 29. Synthesis of Compound 29

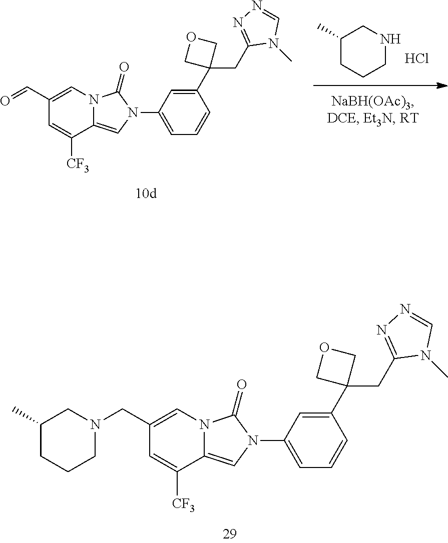

### Synthesis of 29

To a stirred mixture of 10d (100.00 mg, 0.219 mmol, 1.00 equiv) and (3S)-3-methylpiperidine hydrochloride (5931 mg, 0.438 mmol, 2.00 equiv) in DCE (2.00 mL) were added Et₃N (44.24 mg, 0.438 mmol, 2.00 equiv) and NaBH(OAc)₃ (92.67 mg, 0,438 mmol, 2.00 equiv). The resulting mixture was stirred for overnight at room temperature. The resulting mixture was diluted with water (10 mL). The aqueous layer was extracted with CH₂Cl₂/MeOH=10/1 (3×15 mL). The resulting mixture was concentrated under vacuum. The crude product was purified by prep-HPLC with the following conditions (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 30% B to 60% B in 8 min, 60% B; Wave Length: 220 nm; RT1 (min): 7.43) to afford Compound 29 (46.2 mg, 39.09%) as a yellow solid.

LCMS: (ES, m/z): [M+H]⁺541

¹H NMR: (400 MHz, DMSO-d6, ppm): δ 0.82-0.83 (d, 4H), 1.30-1.51 (m, 1H), 1.52-1.72 (m, 4H), 1.82-1.91 (m, 1H), 2.65-2.83 (m, 2H), 2.97 (s, 3H), 3.24 (s, 2H), 3.53 (s, 2H), 4.90-4.92 (d, 2H), 4.94-4.96 (d, 2H), 6.88-6.90 (d, 1H), 7.00 (s, 1H), 7.38 (s, 1H), 7.40-7.42 (m, 2H), 7.65 (s, 1H), 7.74-7.76 (m, 1H), 8.20 (s, 1H).

Alternatively, Compound 20 may be also prepared in the manner outlined below:

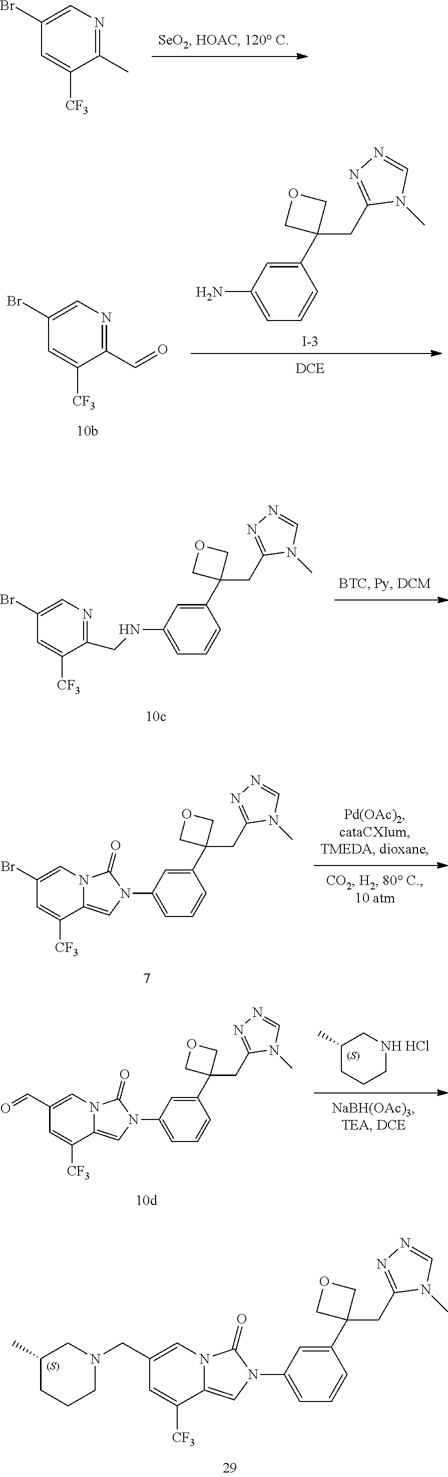

### 1. Synthesis of 10b

A mixture of 5-bromo-2-methyl-3-(trifluoromethyl)pyridine (100 g, 416.627 mmol, 1 equiv) and SeO₂ (92.47 g, 833.254 mmol, 2 equiv) in AcOH (500 mL) was stirred for overnight at 120° C. The resulting mixture was diluted with water (1000 mL). The aqueous layer was extracted with methyl tert-butyl ether (2×500 mL). The resulting mixture was concentrated under reduced pressure. The residue was purified by trituration with hexane (100 mL). This resulted in 5-bromo-3-(trifluoromethyl)pyridine-2-carbaldehyde (62 g, 58.81%) as a off-white solid.

### 2. Synthesis of 10c

To a stirred solution of 10b (100 g, 395.26 mmol, 1.20 equiv) and I-3 (80 g, 329.38 mmol, 1.00 equiv) in DCE (1000.00 mL) were added NaBH(OAc)₃ (139.65 g, 658.761 mmol, 3.00 equiv) at room temperature under nitrogen atmosphere. The resulting mixture was stirred for overnight at room temperature. The resulting mixture was diluted with water (2500.00 mL). The aqueous layer was extracted with EtOAc (3×1000.00 mL). The organic layers were concentrated under reduced pressure. The residue was purified by trituration with MTBE (2×300.00 mL). This resulted in 10c (116 g, 61.01%) as a white solid.

### 3. Synthesis of 7

To a stirred solution of 10c (130 g, 269.539 mmol, 1 equiv) and Pyridine (127.92 g, 1617.234 mmol, 6 equiv) in DCM (2600 mL) was added Triphosgene (26.39 g, 94.594 mmol, 0.35 equiv) at 0° C. The resulting mixture was stirred for 1 h at 0° C. The reaction was quenched by the addition of NaHCO₃ (aq.) (1500 mL). The resulting mixture was extracted with CH₂Cl₂/MeOH=10/1 (2×300 mL). The combined organic layers were dried over anhydrous Na₂SO₄. After filtration, the filtrate was concentrated under reduced pressure. The residue was purified by trituration with methyl tert-butyl ether (600 mL). This resulted in 7 (110 g, 80.29%) as a yellow solid.

(ES, m/z): [M+H]⁺: 508

### 4. Synthesis of 10d

To a solution of 7 (110 g, 216.408 mmol, 1 equiv), TMEDA (50.30 g, 432.816 mmol, 2 equiv) in dioxane (4400 mL) was added bis(adamantan-1-yl)(butyl)phosphane (15.52 g, 43.282 mmol, 0.2 equiv) and Pd(OAc)2 (4.86 g, 21.641 mmol, 0.1 equiv) in an autoclave. After flushing the autoclave three times with CO/H₂ (1:1), the mixture was pressurized to 10 atm with CO/H₂ (1:1) at 80 degrees overnight. The resulting mixture was concentrated under vacuum. The residue was purified by silica gel column chromatography, eluted with CH₂Cl₂/MeOH (20:1) to afford 10d (58 g, 58.59%) as a yellow solid.

(ES, m/z): [M+H]⁺: 458

### 5. Synthesis of 29

To a stirred mixture of 10d (58 g, 126.800 mmol, 1.00 equiv) and (s)-3-methylpiperidine hydrochloride (34.40 g, 253.600 mmol, 2 equiv) in DCE (1600 mL) was added TEA (38.49 g, 380.400 mmol, 3 equiv). The resulting mixture was stirred for 2 h at room temperature. To the above mixture was added NaBH(OAc)3 (53.75 g, 253.600 mmol, 2 equiv). The resulting mixture was stirred overnight at room temperature. The reaction was quenched by the addition of Water (1000 mL). The resulting mixture was extracted with CH₂Cl₂/MEOH=10/1 (2×1000 mL). The combined organic layers were concentrated under reduced pressure. The residue was purified by reverse flash chromatography with the following conditions: column, C18 silica gel; mobile phase, MeCN in water (10 mmoL/L NH₄HCO₃), 15% to 80% gradient in 40 min; detector, UV 254 nm. This resulted in 29 (30.9 g, 45.08%) as a yellow solid. LC-MS-29: (ES, m/z): [M+H]⁺ 541. H-NMR-29: (400 MHz, CD₃OD, δ ppm): 0.75-0.95 (m, 4H), 1.43-1.49 (m, 1H), 1.49-1.66 (m, 4H), 1.86-1.91 (m, 1H), 2.67-2.76 (m, 2H), 2.96 (s, 3H), 3.31 (s, 2H), 3.53 (s, 2H), 4.90-4.95 (m, 1H), 6.88-9.90 (d, 1H), 7.00 (s, 1H), 7.29 (s, 1H), 7.37-7.40 (d, 1H), 7.64 (s, 1H), 7.74-7.76 (d, 1H), 8.19 (s, 1H).

### Example 30. Synthesis of Compound 30

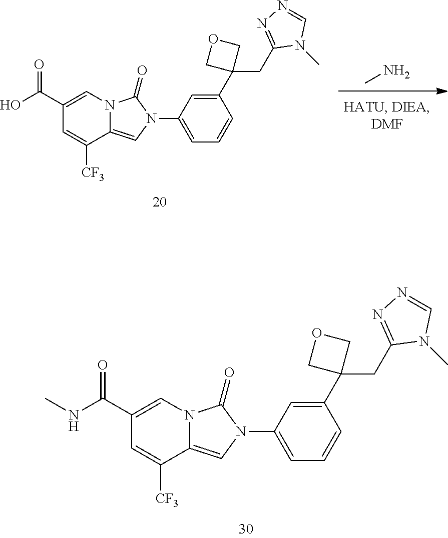

### Synthesis of 30

To a stirred solution of Compound 20 (180.00 mg, 0.380 mmol, 1.00 equiv), methylamine (0.57 mL, 1.140 mmol, 3.00 equiv, 2M in THF) and DIEA (147.42 mg, 1.141 mmol, 3.00 equiv) in DMF (2.00 mL) was added HATU (289.14 mg, 0.760 mmol, 2.00 equiv) at room temperature. The resulting mixture was stirred overnight at room temperature. The crude product was purified by prep-HPLC with the following conditions (Column: XBridge Prep OBD C18 Column, 30*150 mm, 5 m; Mobile Phase A: Water (10 mmol/L NH4HCO3), Mobile Phase B: ACN; Flow rate: 60 mL/min; Gradient: 15% B to 40% B in 8 min, Wave Length: 220 nm; RT1 (min): 6.82) to afford Compound 30 (34.5 mg, 18.65%) as a yellow solid.

LCMS: (ES, m/z): [M+H]⁺ 487

¹H NMR: (400 MHz, DMSO-d6, ppm): δ 2.88 (s, 3H), 2.97 (s, 3H), 3.68 (s, 2H), 5.05-5.10 (m, 4H), 6.91-6.93 (d, 1H), 7.18 (s, 1H), 7.33 (s, 1H), 7.42 (s, 1H), 7.46-7.50 (m, 1H), 7.63-7.65 (d, 1H), 8.20 (s, 1H), 8.38 (s, 1H).

### Example 31. Synthesis of Compound 31

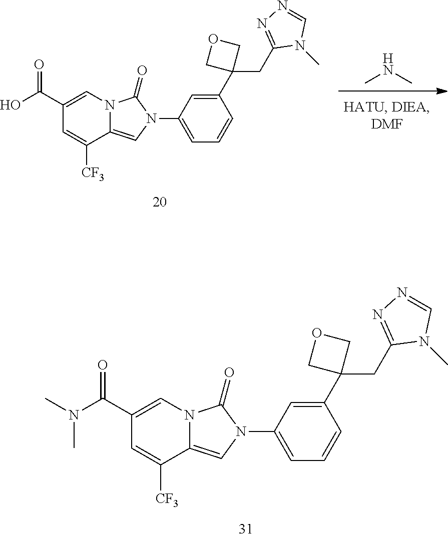

### Synthesis of 31

To a stirred solution of Compound 20 (180.00 mg, 0.380 mmol, 1.00 equiv), dimethylamine (0.57 mL, 1.140 mmol, 3.00 equiv, 2M in THF) and DIEA (147.42 mg, 1.140 mmol, 3.00 equiv) in DMF (2.00 mL) was added HATU (289.14 mg, 0.760 mmol, 2.00 equiv) at room temperature. The resulting mixture was stirred overnight at room temperature. The reaction solution was purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 15% B to 35% B in 8 min; Wave Length: 220 nm; RT1 (min): 7.77) to afford Compound 31 (45 mg, 23.65%) as a yellow solid.

LCMS: (ES, m/z): [M+H]⁺ 501

¹H NMR: (400 MHz, DMSO-d6, ppm): δ 2.99 (s, 6H), 3.03 (s, 3H), 3.54 (s, 2H), 4.91-4.97 (m, 4H), 6.90-6.92 (d, 1H), 7.08 (s, 1H), 7.40-7.45 (m, 3H), 7.74-7.76 (m, 1H), 7.90 (s, 1H), 8.20 (s, 1H).

### Example 32. Synthesis of Compound 32

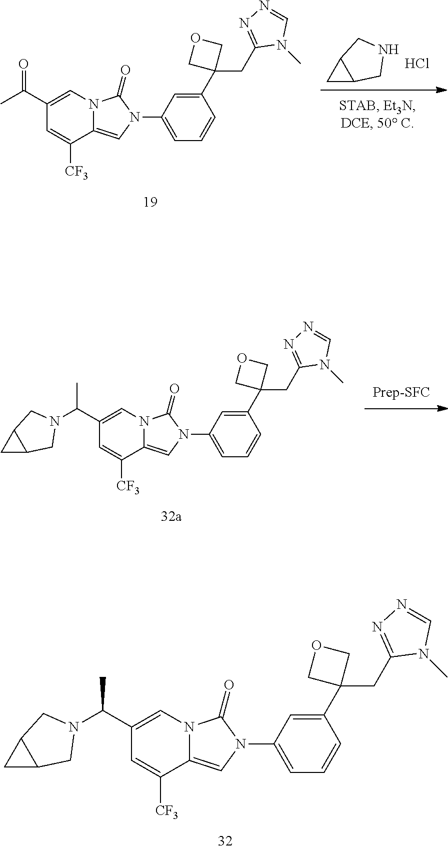

### Synthesis of 32a

To a stirred mixture of 32a (400.00 mg, 0.848 mmol, 1.00 equiv) and 3-azabicyclo[3.1.0]hexane hydrochloride (152.20 mg, 1.273 mmol, 1.5 equiv) in DCE (5.00 mL) were added STAB (539.47 mg, 2.545 mmol, 3 equiv) and Et₃N (257.57 mg, 2.545 mmol, 3 equiv) at room temperature. The resulting mixture was stirred overnight at 50° C., The reaction was quenched by the addition of NH₄Cl (aq.) (20 mL) at room temperature. The aqueous layer was extracted with CH₂Cl₂ (3×20 mL). The resulting mixture was concentrated under reduced pressure. The residue was purified by prep-TLC (CH₂Cl₂/MeOH 10:1) to afford 32a (45 mg, 9.65%) as a yellow solid.

### Synthesis of 32

The 32a (45 mg) was separated by prep-SFC with the following conditions (Column: Lux 5 μm Cellulose-4, 3*25 cm, 5 n; Mobile Phase A: CO₂, Mobile Phase B: MEOH (0.1% 2M NH₃-MEOH); Flow rate: 80 mL/min; Gradient: isocratic 50% B; Column Temperature (° C.): 35; Back Pressure (bar): 100; Wave Length: 254 nm; RT2 (min): 13.78) to afford Compound 32 (4.3 mg) as a yellow solid.

LCMS: (ES, m/z): [M+H]⁺ 539

¹H NMR (400 MHz, DMSO, δ ppm): δ 0.31-0.37 (d, 1H), 0.59-0.66 (d, 1H), 1.20-1.27 (m, 3H), 1.31-1.37 (d, 1H), 1.38-1.42 (m, 1H), 2.20-2.31 (m, 1H), 2.32-2.43 (m, 1H), 2.61-2.71 (d, 1H), 2.97 (s, 3H), 3.00-3.04 (d, 1H), 3.25-3.30 (d, 1H), 3.50-3.55 (d, 2H), 4.87-5.05 (m, 4H), 6.80-6.95 (d, 1H), 6.97 (s, 1H), 7.31 (s, 1H), 7.38-7.51 (m, 2H), 7.63 (s, 1H), 7.70-7.80 (m, 1H), 8.20 (s, 1H).

### Example 33. Synthesis of Compound 33

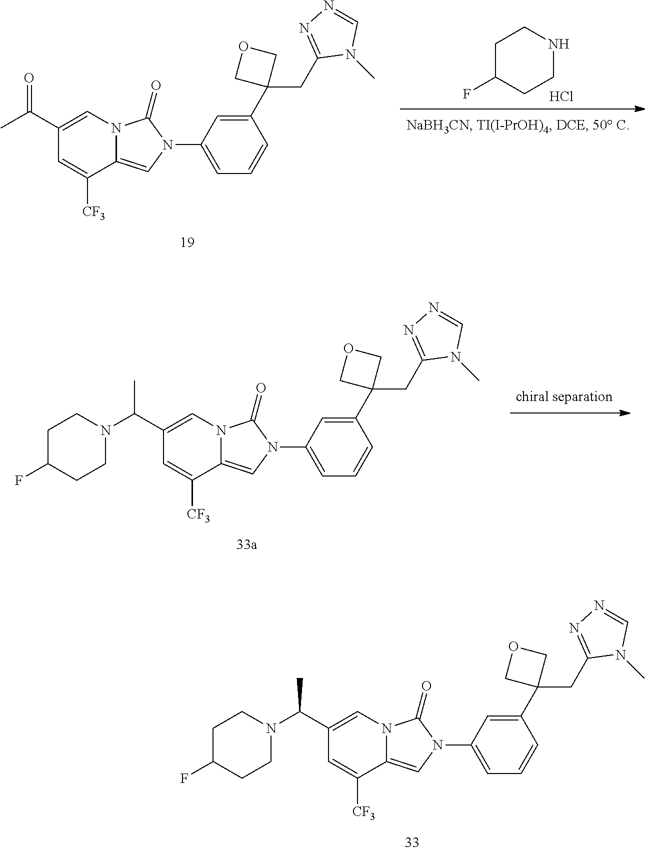 [SMILES: CC(=O)c1cc(C(F)(F)F)c2cn(-c3cccc(C4(Cc5nncn5C)COC4)c3)c(=O)n2c1 ; FC1CCNCC1 ; Cl ; CC(c1cc(C(F)(F)F)c2cn(-c3cccc(C4(Cc5nncn5C)COC4)c3)c(=O)n2c1)N1CCC(F)CC1 ; C[C@@H](c1cc(C(F)(F)F)c2cn(-c3cccc(C4(Cc5nncn5C)COC4)c3)c(=O)n2c1)N1CCC(F)CC1]

### Synthesis of 33a

To a stirred solution of 19a (200.00 mg, 0.424 mmol, 1.00 equiv) and 4-fluoropiperidine hydrochloride (59.22 mg, 0.424 mmol, 1.00 equiv) in DCE (2.00 mL) were added Ti(Oi-Pr)₄ (241.15 mg, 0.848 mmol, 2.00 equiv) and NaBH₃CN (31.99 tug, 0.509 mmol, 1.20 equiv) at room temperature under nitrogen atmosphere. The resulting mixture was stirred overnight at 50° C. The resulting mixture was diluted with water (30 mL) at room temperature. The aqueous layer was extracted with EtOAc (2×10 mL). The residue was purified by prep-TLC (CH₂Cl₂/MeOH=20:1) to afford 33a (40 mg, 16.88%) as a yellow solid.

### Synthesis of 33

The compound of 33a (40 mg) was purified by prep-Chiral HPLC with the following conditions (Column: CHIRALPAK IC, 2*25 cm, 5 μm; Mobile Phase A: Hex (0.5% 2M NH₃-MeOH), Mobile Phase B: MeOH:DCM=1:1; Flow rate: 20 mL/min; Gradient: 55% B to 55% B in 18 min; Wave Length: 220 nm; RT2 (min): 16.44) to afford Compound 33 (4.4 mg) as a yellow solid.

LCMS: (ES, m/z): [M+H]⁺ 559.

¹H NMR: (400 MHz, DMSO, δ ppm): δ 1.24-1.28 (m, 3H), 1.69-1.80 (m, 2H), 1.80-1.91 (m, 2H), 2.33-2.37 (m, 2H), 2.58-2.68 (m, 2H), 2.97 (s, 3H), 3.49-3.53 (m, 3H), 4.59-4.73 (m, 1H), 4.83-4.96 (m, 4H), 6.89-6.91 (m, 1H), 7.08 (s, 1H), 7.31-7.38 (m, 1H), 7.40-7.42 (m, 1H), 7.60-7.61 (m, 1H), 7.73-7.76 (m, 1H), 8.20 (s, 1H).

### Example 34. Synthesis of Compound 34

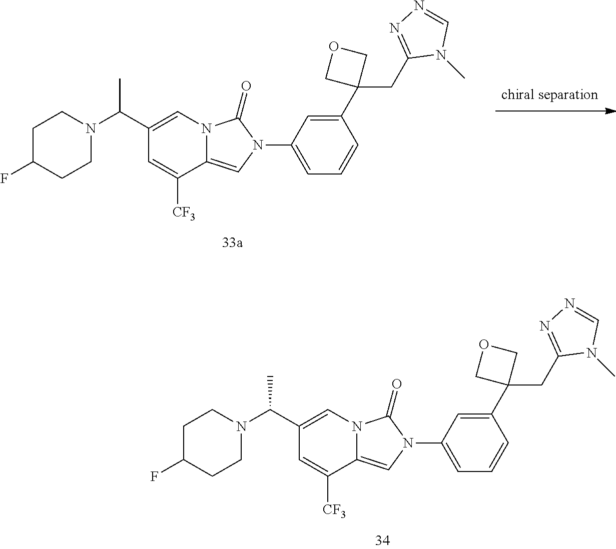 [SMILES: CC(c1cc(C(F)(F)F)c2cn(-c3cccc(C4(Cc5nncn5C)COC4)c3)c(=O)n2c1)N1CCC(F)CC1 ; C[C@H](c1cc(C(F)(F)F)c2cn(-c3cccc(C4(Cc5nncn5C)COC4)c3)c(=O)n2c1)N1CCC(F)CC1]

### Synthesis of 34

The compound of 33a (40 mg) was purified by prep-Chiral HPLC with the following conditions (Column: CHIRALPAK IC, 2*25 cm, 5 μm; Mobile Phase A: Hex (0.5% 2M NH₃-MeOH), Mobile Phase B: MeOH:DCM=1:1; Flow rate: 20 mL/min; Gradient: 55% B to 55% B in 18 min; Wave Length: 220 nm; RT1 (min): 14.61) to afford Compound 34 (5.2 mg) as a yellow solid.

LCMS: (ES, m/z): [M+H]⁺ 559

¹H NMR (400 MHz, DMSO, δ ppm): δ 1.22-1.28 (m, 3H), 1.69-1.80 (m, 2H), 1.81-1.87 (m, 2H), 2.34-2.36 (m, 2H), 2.57-2.68 (m, 2H), 2.87-2.88 (m, 1H), 2.97 (s, 2H), 3.48-3.53 (m, 3H), 4.60-4.72 (m, 1H), 4.82-4.96 (m, 4H), 6.89-6.91 (m, 1H), 7.08-7.09 (m, 1H), 7.31-7.38 (m, 1H), 7.40-7.42 (m, 2H), 7.60-7.61 (m, 1H), 7.74-7.76 (m, 1H), 8.20 (s, 1H).

### Example 35. Synthesis of Compound 35

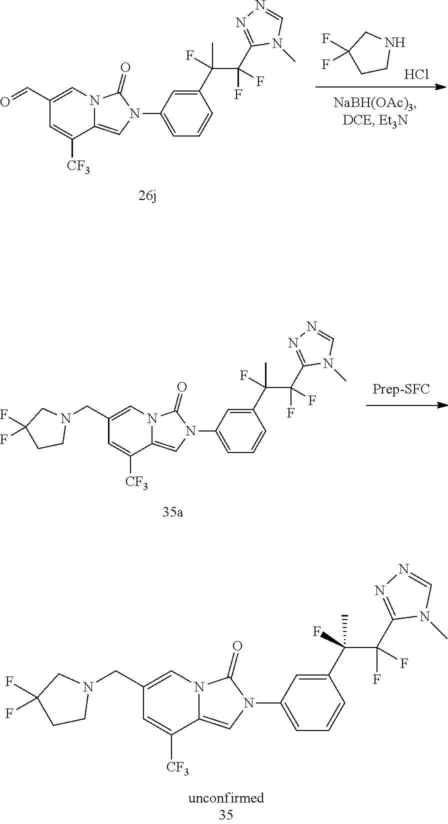

### Synthesis of 35a

To a stirred solution of 26j (250.00 mg, 0.517 mmol, 1.00 equiv) and 3,3-difluoropyrrolidine hydrochloride (222.75 mg, 1.552 mmol, 3.00 equiv) in DCE (10.00 mL) was added Et₃N (157.01 mg, 1.552 mmol, 3 equiv) at room temperature under nitrogen atmosphere. The resulting mixture was stirred for 10 min at room temperature. To the above mixture was added NaBH(OAc)₃ (328.85 mg, 1.552 mmol, 3 equiv), and then the mixture was stirred overnight at room temperature. The resulting mixture was washed with 10 ml, of water. The residue was purified by prep-TLC (CH₂Cl₂/MeOH 20:1) to afford 35a (220 mg, 69.60%) as a yellow solid.

### Synthesis of 35

The crude product (35a, 220 mg) was purified by prep-SFC with the following conditions (Column: CHIRALPAK IF, 3*25 cm, 5 μm; Mobile Phase A: CO₂, Mobile Phase B: MeOH (0.5% 2M NH₃MeOH)-HPLC; Flow rate: 100 mL/min; Gradient: isocratic 25% B; Back Pressure (bar): 100; Wave Length: 220 am; RT1 (min): 5.6; Sample Solvent: MeOH; Injection Volume: 1.8 mL; Number Of Runs: 11) to afford Compound 35 (31.9 mg, 14.38%) as a yellow solid.

LCMS: (ES, m/z): [M+H]⁺ 575

¹H NMR: (400 MHz, CD₃OD-d4, ppm): δ 2.05-2.08 (m, 3H), 2.26-2.37 (m, 2H), 2.82-2.84 (m, 2H), 2.93-3.00 (m, 2H), 3.47 (s, 2H), 3.65 (s, 3H), 7.10 (s, 1H), 7.15 (s, 1H), 7.43-7.45 (d, 1H), 7.60-7.62 (m, 1H), 7.72 (s, 1H), 7.76-7.80 (m, 2H), 8.54 (s, 1H).

### Example 36. Synthesis of Compound 36

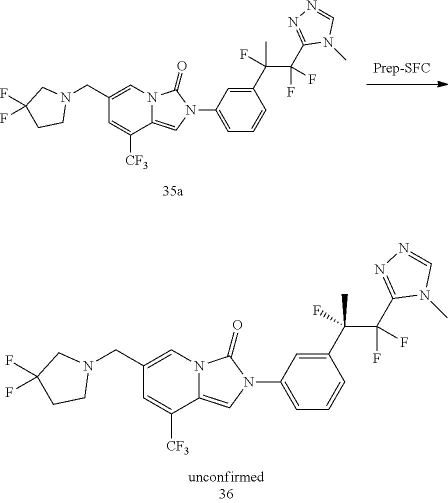 [SMILES: Cn1cnnc1C(F)(F)C(C)(F)c1cccc(-n2cc3c(C(F)(F)F)cc(CN4CCC(F)(F)C4)cn3c2=O)c1 ; Cn1cnnc1C(F)(F)[C@@](C)(F)c1cccc(-n2cc3c(C(F)(F)F)cc(CN4CCC(F)(F)C4)cn3c2=O)c1]

### Synthesis of 36

The crude product (35a, 220 mg) was purified by prep-SFC with the following conditions (Column: CHIRALPAK IF, 3*25 cm, 5 μM; Mobile Phase A: CO, Mobile Phase B: MeOH (0.5% 2M NH₃)-HPLC; Flow rate: 100 mL/min; Gradient: isocratic 25% B; Column Temperature ° C.): 35; Back Pressure (bar): 100; Wave Length: 220 nm; RT2 (min): 6.82) to afford Compound 36 (78.3 mg, 34.17%) as a yellow solid.

LCMS: (ES, m/z): [M+H]⁺ 575

¹H NMR: (400 MHz, CD₃OD-d4, ppm): δ 1.99-2.08 (m, 3H), 2.26-2.37 (m, 2H), 2.81-2.84 (m, 2H), 2.93-3.00 (m, 2H), 3.48 (s, 2H), 3.65 (s, 3H), 7.10 (s, 1H), 7.14 (s, 1H), 7.42-7.47 (d, 1H), 7.57-7.61 (m, 1H), 7.72 (s, 1H), 7.76-7.82 (m, 2H), 8.53 (s, 1H).

### Example 37. Synthesis of Compound 37

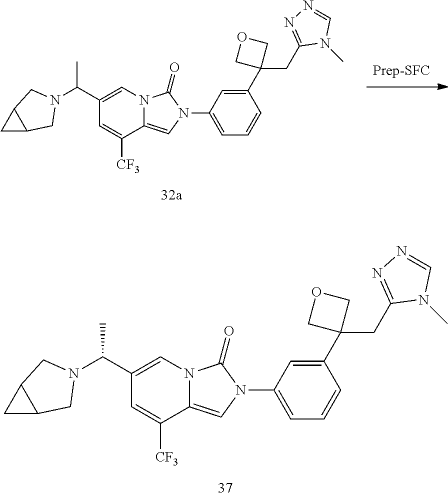 [SMILES: CC(c1cc(C(F)(F)F)c2cn(-c3cccc(C4(Cc5nncn5C)COC4)c3)c(=O)n2c1)N1CC2CC2C1 ; C[C@H](c1cc(C(F)(F)F)c2cn(-c3cccc(C4(Cc5nncn5C)COC4)c3)c(=O)n2c1)N1CC2CC2C1]

### Synthesis of 37

The compound of 32a (45 mg) was separated by prep-SFC with the following conditions (Column: Lux 5 μm Cellulose-4, 3*25 cm, 5 μm; Mobile Phase A: CO₂, Mobile Phase B: MEOH (0.1% 2M NH₃-MEOH); Flow rate: 80 mL/min; (Gradient: isocratic 50% B; Column Temperature (° C.): 35; Back Pressure (bar): 100; Wave Length: 254 nm; RT1 (min): 11.52) to afford Compound 37 (3.3 mg) as a yellow solid.

LCMS: (ES, m/z): [M+H]⁺ 539

¹H NMR: (400 MHz, DMSO, δ ppm): δ 0.26-0.40 (d, 1H), 0.57-0.66 (d, 1H), 1.22-1.27 (t, 3H), 1.31-1.37 (d, 1H), 1.38-1.45 (m, 1H), 2.19-2.31 (m, 1H), 2.35-2.44 (m, 1H), 2.61-2.71 (d, 1H), 2.95-2.98 (d, 2H), 3.00-3.07 (m, 1H), 3.25-3.30 (d, 1H), 3.41-3.50 (d, 1H), 3.51-3.61 (d, 2H), 4.87-5.05 (m, 4H), 6.88-6.90 (d, 1H), 6.97 (s, 1H), 7.30 (s, 1H), 7.38-7.42 (m, 2H), 7.62 (s, 1H), 7.73-7.76 (m, 1H), 8.20 (s, 1H).

### Example 38. Synthesis of Compound 38

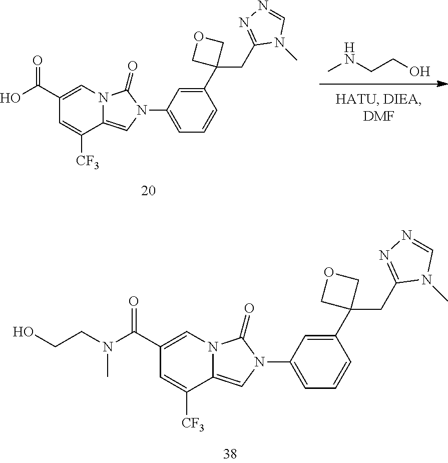

### Synthesis of 38

To a stirred solution of Compound 20 (180.00 mg, 0.380 mmol, 1.00 equiv) and methylethanolamine (57.12 mg, 0.760 mmol, 2.00 equiv) in DMF (2.00 mL) were added DIEA (98.28 mg, 0.760 mmol, 2.00 equiv) and DIEA (98.28 mg, 0.760 mmol, 2.00 equiv). The resulting mixture was stirred for 2 h at room temperature. The reaction mixture was purified by prep-HPLC with the following conditions (Column: XBridge Prep OBD C18 Column, 30*150 mm, 5 m; Mobile Phase A: Water (10 mmol/L NH4HCO3), Mobile Phase B: ACN; Flow rate: 60 mL/min; Gradient: 10% B to 35% B in 8 min, 35% B; Wave Length: 220 nm; RT1 (min): 7.50) to afford Compound 38 (34.3 mg, 17.00%) as a yellow solid.

LCMS: (ES, m/z): [M+H]⁺ 531

¹H NMR: (400 MHz, DMSO-d6, ppm): δ 2.86-3.01 (m, 6H), 3.44-3.49 (m, 2H), 3.54 (s, 2H), 3.58-3.62 (m, 2H), 4.89-4.97 (m, 4H), 6.89-6.91 (d, 1H), 7.09 (s, 1H), 7.39-7.45 (m, 3H), 7.74-7.76 (m, 1H), 8.09 (s, 1H), 8.21 (s, 1H).

### Example 39 Synthesis of Compound 39

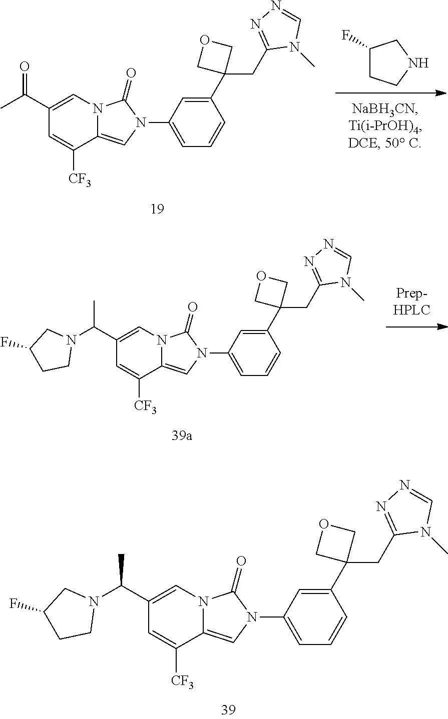 [SMILES: CC(=O)c1cc(C(F)(F)F)c2cn(-c3cccc(C4(Cc5nncn5C)COC4)c3)c(=O)n2c1 ; F[C@H]1CCNC1 ; CC(c1cc(C(F)(F)F)c2cn(-c3cccc(C4(Cc5nncn5C)COC4)c3)c(=O)n2c1)N1CC[C@H](F)C1 ; C[C@@H](c1cc(C(F)(F)F)c2cn(-c3cccc(C4(Cc5nncn5C)COC4)c3)c(=O)n2c1)N1CC[C@H](F)C1]

### Synthesis of 39a

To a stirred mixture of Compound 19 (150.00 mg, 0.318 mmol, 1.00 equiv) and (3S)-3-fluoropyrrolidine (42.53 mg, 0.477 mmol, 1.5 equiv) in DCE (2.00 mL) were added NaBH₃ClN (59.98 mg, 0.955 mmol, 3 equiv) and titanium(IV) isopropoxide (90.43 mg, 0,318 mmol, 1 equiv) dropwise at room temperature. The resulting mixture was stirred for 4 h at 50° C. under nitrogen atmosphere. The reaction was quenched by the addition of NH₄Cl (aq.) (20 mL) at room temperature. The aqueous layer was extracted with CH₂Cl₂ (3×20 mL). The resulting mixture was concentrated under reduced pressure. The residue was purified by prep-TLC (CH₂Cl₂/MeOH 10:1) to afford 39a (48 mag, 26.32%) as a yellow solid,

### Synthesis of 39

The 39a (48 mg) was separated by prep-HPLC with the following conditions (Column: YMC-Actus Triart C18 ExRS, 30*150 mm, 5 um; Mobile Phase A: Water (10 mmol/L NH₄HCO₃), Mobile Phase B: ACN; Flow rate: 60 mL/min; Gradient: 35% B to 40% B in 7 min, 40% B; Wave Length: 254 nm; RT1 (min): 7.48) to afford Compound 39 (4.4 mg) as a yellow solid,

LCMS: (ES, m/z): [M+H]⁺ 545

¹H NMR: (400 MHz, DMSO, ppm): δ 1.23-1.36 (d, 3H), 1.76-2.00 (m, 1H), 2.00-2.21 (m, 1H), 2.37-2.47 (d, 1H), 2.58-2.71 (m, 2H), 2.77-2.96 (m, 1H), 2.96-3.01 (s, 3H), 3.27-3.31 (t, 1H), 3.45-3.57 (s, 2H), 4.85-5.06 (m, 4H), 5.10-5.39 (d, 1H), 6.89-6.94 (d, 1H), 7.05 (s, 1H), 7.27-7.36 (d, 1H), 7.36-7.48 (m, 2H), 7.70 (s, 1H), 7.74-7.76 (d, 1H), 8.20 (s, 1H).

### Example 40. Synthesis of Compound 40

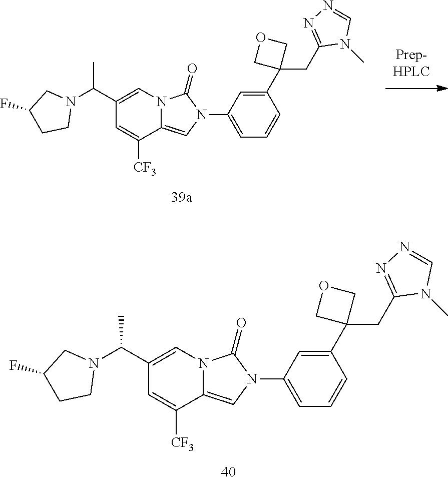 [SMILES: CC(c1cc(C(F)(F)F)c2cn(-c3cccc(C4(Cc5nncn5C)COC4)c3)c(=O)n2c1)N1CC[C@H](F)C1 ; C[C@H](c1cc(C(F)(F)F)c2cn(-c3cccc(C4(Cc5nncn5C)COC4)c3)c(=O)n2c1)N1CC[C@H](F)C1]

### Synthesis of 40

Compound 39a (48 mg) was separated by prep-HPLC with the following conditions (Column: YMC-Actus Triart C18 ExRS, 30*150 mm, 5 pin; Mobile Phase A: Water (10 mmol/L NH₄HCO₃), Mobile Phase B: ACN; Flow rate: 60 mL/min; Gradient: 35% B to 40% B in 7 min, 40% B; Wave Length: 254 nm; RT1 (min): 7.48) to afford Compound 40 (3.0 mg) as a yellow solid.

LCMS: (ES, m/z): [M+H]⁺ 545

¹H NMR: (400 MHz, DMSO, ppm): δ 1.27-1.36 (d, 3H), 1.78-2.01 (m, 1H), 2.00-2.22 (m, 1H), 2.22-2.34 (d, 1H), 2.58-2.80 (m, 2H), 2.80-2.95 (m, 1H), 2.95-3.01 (s, 3H), 3.23-3.31 (t, 1H), 3.53-3.57 (s, 2H), 4.85-5.00 (m, 4H), 5.05-5.32 (d, 1H), 6.82-6.92 (d, 1H), 7.06 (s, 1H), 7.32-7.36 (s, 1H), 7.40-7.42 (m, 2H), 7.70 (s, 1H), 7.74-7.76 (m, 1H), 8.20 (s, 1H).

### Example 41. Synthesis of Compound 41

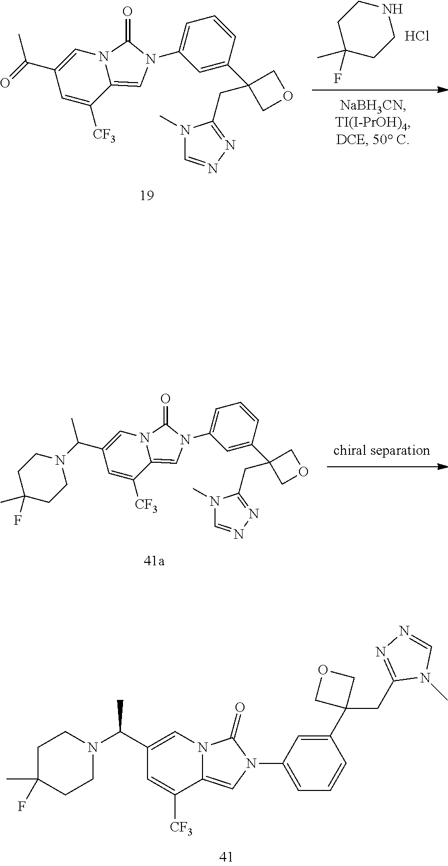

### Synthesis of 41a

To a stirred solution of Compound 19 (60.00 mg, 0,552 mmol, 1.00 equiv) and 4-fluoro-4-methylpiperidine hydrochloride (84.73 mg, 0.552 mmol, 1.00 equiv) in DCE (3.00 mL) were added Ti(Oi-Pr)₄ (156.75 ng, 0.552 mmol, 1.00 equiv) and NaBH₃CN (41.59 mg, 0.662 mmol, 1.20 equiv) at room temperature. The resulting mixture was stirred for overnight at 50° C. The resulting mixture was diluted with water (20 mL) at room temperature. The aqueous layer was extracted with CH₂Cl₂ (3×20 mL). The residue was purified by prep-TLC (CH₂Cl₂/MeOH=20:1) to afford 41a (50 mg) as a yellow solid. The crude product (50 mg) was purified by prep-HPLC with the following conditions (Column: XBridge Prep OBD C18 Column, 30*150 mm, 5 n; Mobile Phase A: water (10 mmol/L NH₄HCO₃), Mobile Phase B: ACN; Flow rate: 60 mL/min; Gradient: 30% B to 55% B in 8 min, 55% B; Wave Length: 220 nm; RT1 (min): 7.55; Number Of Runs: 0) to afford 41a (20 mg, 6.33%) as a yellow solid.

### Synthesis of 41

The compound of 41 a (20 mg) was separated by prep-Chiral-HPLC with the following conditions (Column: CHIRALPAK TC, 2*25 cm, 5 μm; Mobile Phase A: Hex (0.5% 2M NH₃-MeOH), Mobile Phase B: MeOH:DCM=1:1; Flow rate: 20 mL/min; Gradient: 55% B to 55% B in 15 min; Wave Length: 220 nm; RT2 (min): 13.73) to afford Compound 41 (6.9 mg) as a yellow solid.

LCMS: (ES, m/z): [M+H]⁺ 573

¹H NMR (400 MHz, DMSO, δ ppm): δ 1.22-1.38 (m, 6H), 1.55-1.63 (m, 1H), 1.63-1.74 (m, 3H), 2.25-2.36 (m, 2H), 2.60-2.67 (m, 2H), 2.97 (s, 3H), 3.47-3.53 (m, 3H), 4.91-4.96 (m, 4H), 6.89-6.91 (m, 1H), 7.07-7.08 (m, 1H), 7.31-7.32 (m, 1H), 7.38-7.42 (m, 2H), 7.60-7.61 (m, 1H), 7.73-7.76 (m, 1H), 8.20 (s, 1H).

### Example 42. Synthesis of Compound 42

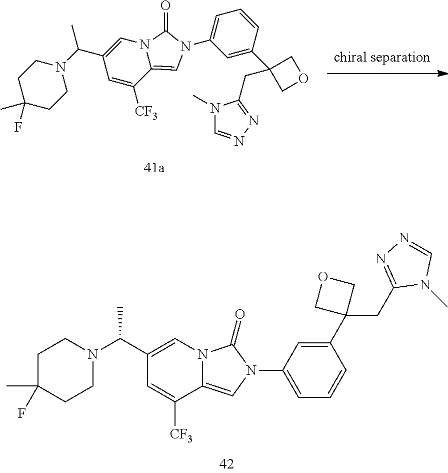

### Synthesis of 42

The compound of 41a (20 mg) was separated by prep-Chiral-HPLC with the following conditions (Column: CHIRALPAK IC, 2*25 cm, 5 n; Mobile Phase A: Hex (0.5% 2M NH₃-MeOH), Mobile Phase B: MeOH:DCM=1:1; Flow rate: 20 mL/min; Gradient: 55% B to 55% B in 15 min; Wave Length: 220 nm; RT1 (min): 12.42) to afford Compound 42 (6.6 mg) as a yellow solid.

LCMS: (ES, m/z): [M+H]⁺ 573

¹H NMR (400 MHz, DMSO, δ ppm): δ 1.24-1.33 (m, 6H), 1.59-1.62 (m, 1H), 1.71-1.74 (m, 3H), 2.29-2.33 (m, 2H), 2.61-2.67 (m, 2H), 2.97 (s, 3H), 3.47-3.53 (m, 3H), 4.91-4.96 (m, 4H), 6.89-6.91 (m, 1H), 7.08-7.08 (m, 1H), 7.31-7.32 (m, 1H), 7.38-7.42 (m, 2H), 7.61-7.62 (m, 1H), 7.73-7.76 (m, 1H), 8.20 (s, 1H).

### Example 43. Synthesis of Compound 43

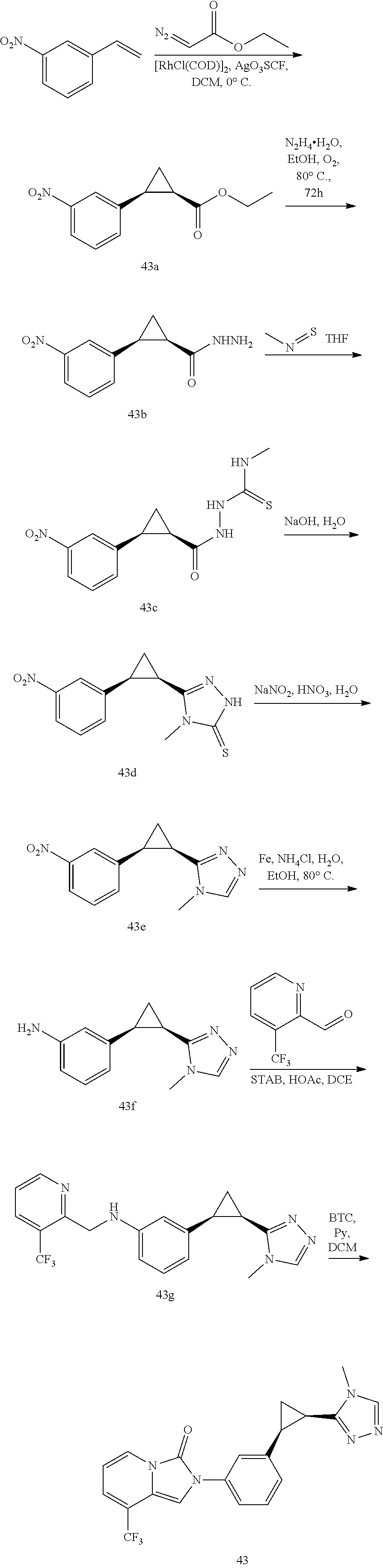

### Synthesis of 43a

To a stirred solution of cyclooctadiene rhodium chloride dimer (3.31 g, 6.713 mmol, 0.10 equiv) in DCM (150.00 mL) were added silver triflate (1.72 g, 6.694 mmol, 0.10 equiv) at 0° C. under nitrogen atmosphere. The resulting mixture was stirred for 30 min at 0° C. To the above mixture was added ethyl diazoacetate (38.25 g, 0.335 mmol, S equiv) and (3-nitrophenyl)ethene (10.00 g, 67.047 mmol, 1.00 equiv) at 0° C. The resulting mixture was stirred overnight at room temperature. The resulting mixture was washed with 1×100 mL of water. The residue was purified by silica gel column chromatography, eluted with PE/EtOAc (10:1) to afford the crude product. The residue was purified by prep-TLC (PE/EtOAc 8:1) to afford 43a (11 g, 6.63%) as a light yellow oil.

### Synthesis of 43b

To a stirred solution of 43a (1.10 g, 4.676 mmol, 1.00 equiv) in EtOH (20.00 mL) was added hydrazine hydrate (98%) (3.51 g, 70.140 mmol, 15.00 equiv) at room temperature under nitrogen atmosphere. The resulting mixture was stirred for overnight at 80° C. under nitrogen atmosphere. The resulting mixture was diluted with water (100 mL). The aqueous layer was extracted with CH₂Cl₂/MeOH=1:1 (3×100 ml). The residue was purified by prep-TLC (CH₂Cl₂/MeOH 20:1) to afford 43b (600 mg, 52.20%) as a light yellow oil.

### Synthesis of 43c

To a stirred solution of 43b (600.00 mg, 2.712 mmol, 1.00 equiv) in tetrahydrofuran (10.00 mL) was added methyl isothiocyanate (297.44 mg, 4.068 mmol, 1.50 equiv) in portions at room temperature under nitrogen atmosphere. The resulting mixture was stirred for 3h at room temperature. The resulting mixture was diluted with water (50 mL). The precipitated solid, were collected by filtration. This resulted in 43c (700 mg, 78.92%) as an off-white solid.

### Synthesis of 43d

To a stirred solution of NaOH (92.41 mg, 2.310 mmol, 1.00 equiv) in H₂O (23.00 mL) was added 43c (680.00 mg, 2.310 mmol, 1.00 equiv) at room temperature under nitrogen atmosphere. The resulting mixture was stirred for 3h at room temperature. The precipitated solids were collected by filtration and washed with water (10 mL). This resulted in 43d (600 mg, 84.59%) as an off-white solid.

### Synthesis of 43e

To a stirred solution of 43d (600.00 mg, 2.171 mmol, 1.00 equiv) in H₂O (10.00 mL) was added NaNO₂ (1498.22 mg, 21.715 mmol, 10.00 equiv) and HNO₃ (10.00 ml, 1 M) at 0° C. under nitrogen atmosphere. The resulting mixture was stirred for 2h at 0° C. The mixture was basified to pH 7 with saturated NaHCO₃. The precipitated solids were collected by filtration and washed with water (10 mL). This resulted in 43e (400 mg, 68.63%) as an off-white solid.

### Synthesis of 43f

To a stirred solution of Fe (411.54 mg, 7.369 mmol, 5.00 equiv) and 43e (360.00 mg, 1.474 mmol, 1.00 equiv) in EtOH (10.00 mL) was added NH₄Cl (788.39 mg, 14.739 mmol, 10 equiv) in H₂O (10.00 mL) at room temperature under nitrogen atmosphere. The resulting mixture was stirred overnight at 80° C. under nitrogen atmosphere. The resulting mixture was diluted with water (100 mL). The aqueous layer was extracted with CH₂Cl₂ (3×100 mL). The residue was purified by prep-TLC (CH₂Cl₂/MeOH 20:1) to afford 43f (300 mg, 89.29%) as a light yellow solid.

### Synthesis of 43g

To a stirred solution of 43f (280.00 mg, 1.307 mmol, 1.00 equiv) and 3-(trifluoromethyl)pyridine-2-carbaldehyde (228.83 mg, 1.307 mmol, 1.00 equiv) in DCE (10.00 mL) were added HOAc (156.95 mg, 2.614 mmol, 2 equiv) and STAB (830.86 mg, 3.920 mmol, 3 equiv) at room temperature under nitrogen atmosphere. The resulting mixture was stirred overnight at room temperature. The resulting mixture was washed with 20 mL of water. The residue was purified by prep-TLC (CH₂Cl₂/MeOH 12:1) to afford 43g (400 mg, 75.42%) as a light yellow solid.

### Synthesis of 43

To a stirred solution of 43g (200.00 mg, 0.536 mmol, 1.00 equiv) and pyridine (254.22 mg, 3.214 mmol, 6 equiv) in DCM (10.00 mL) was added triphosgene (55.63 mg, 0.187 mmol, 0.35 equiv) at 0° C. under nitrogen atmosphere. The resulting mixture was stirred for 10 min at room temperature. The resulting mixture was concentrated under reduced pressure. The residue was purified by reverse flash chromatography with the following conditions: column, C18 silica gel; mobile phase, A: water, B: MeCN, 35% to 45% gradient in 15 min; detector, IV 254 nm. This resulted in Compound 43 (112.3 mg, 51.08%) as a yellow solid.

LCMS: (ES, m/z): [M+H]⁺ 400

¹H NMR: (400 MHz, DMSO-d6, ppm): δ 1.52-1.65 (m, 1H), 1.90-1.95 (m, 1H), 2.51-2.57 (m, 1H), 2.66-2.72 (m, 1H), 3.40 (s, 3H), 6.26-6.29 (m, 1H), 6.90-6.92 (d, 1H), 7.08-7.12 (m, 2H), 7.20-7.24 (m, 1H), 7.42-7.43 (m, 1H), 7.54-7.57 (d, 1H), 7.76-7.78 (d, 1H), 8.15 (s, 1H).

### Example 44. Synthesis of Compound 44

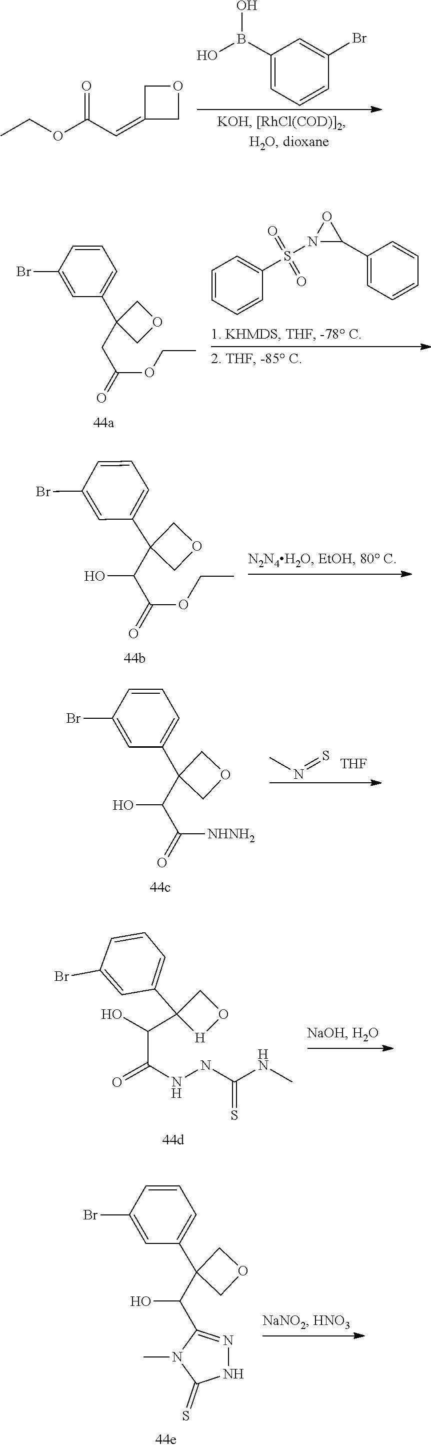

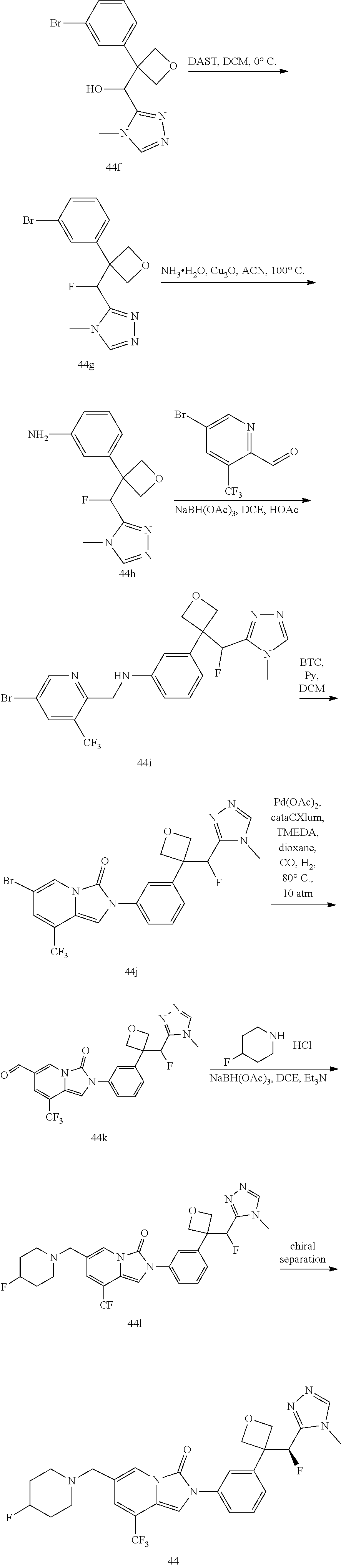

### Synthesis of 44a

To a stirred solution of [Rh(COD)Cl]₂ (700 mg, 0.011 mmol, 0.015 equiv) in dioxane (120 mL) was added KOH (79 mL, 118.182 mmol, 1.2 equiv, 1.5 M) at room temperature. The resulting mixture was stirred for 1h at room temperature under nitrogen atmosphere. To the above mixture was added ethyl 2-(oxetan-3-ylidene)acetate (14.00 g, 98.485 mmol, 1.00 equiv) and 3-bromophenylboronic acid (33.62 g, 167.424 mmol, 17 equiv) at room temperature. The resulting mixture was stirred overnight at room temperature. The reaction was quenched with NH₄Cl (aq.) (500 mL) at room temperature. The aqueous layer was extracted with EtOAc (3×500 mL). The resulting mixture was concentrated under vacuum. The residue was purified by silica gel column chromatography, eluted with PE/EtOAc (5:1) to afford 44a (16 g) as yellow oil,

### Synthesis of 44b

Into a 100 ml, 3-necked round-bottom flask were added 44a (2.00 g, 6,685 mmol, 1.00 equiv), KHMDS (1 mol/L in THF) (10 ml, 10.028 mmol, 1.5 equiv) and THF (20.00 mL) at −78° C. The resulting mixture was stirred for 1h at −78° C. under nitrogen atmosphere. To the above mixture was added 2-(benzenesulfonyl)-3-phenyloxaziridine (2.27 g, 8.691 mmol, 1.3 equiv). The resulting mixture was stirred for additional 3h at −65° C. The resulting mixture was quenched with 60 mL of NH₄Cl (aq.). The aqueous layer was extracted with EtOAc (3×20 mL). The resulting mixture was concentrated under vacuum. The residue was purified by silica gel column chromatography, eluted with PE/EtOAc (5:1) to afford 44b (520 mg, 22.46%) as a yellow oil.

### Synthesis of 44c

Into a 250 mL 3-necked round-bottom flask were added 44b (4.00 g, 12.692 mmol, 1.00 equiv), NH₂NH₂·H₂O (635 g, 126.918 mmol, 10 equiv) and EtOH (40.00 ml, 688.541 mmol, 54.25 equiv) at room temperature. The resulting mixture was stirred for overnight at 80° C. The precipitated solids were collected by filtration and washed with water (3×10 mL) to afford 44c (2.5 g, 58.87%) as an off-white solid.

### Synthesis of 44d

Into a 250 mL 3-necked round-bottom flask were added 44c (2.50 g, 8.302 mmol, 1.00 equiv), methyl isothiocyanate (1.21 g, 16.604 mmol, 2.00 equiv) and tetrahydrofuran (25.00 mL) at room temperature. The resulting mixture was stirred for overnight at room temperature under nitrogen atmosphere. The precipitated solids were collected by filtration and washed with EtOAc (3×10 mL) to afford 44d (2.4 g, 69.52%) as an off-white solid.

### Synthesis of 44e

Into a 100 ml, 3-necked round-bottom flask were added 44d (2.00 g, 5,344 mmol, 1.00 equiv), NaOH (0.43 g, 10.688 mmol, 2 equiv) and HO (20.00 mL) at room temperature. The resulting mixture was stirred overnight at room temperature under nitrogen atmosphere. The residue was neutralized to pH 7 with HCl (1 M). The aqueous layer was extracted with EtOAc (3×10 mL). The resulting mixture was concentrated under vacuum to afford 44e (1.9 g, 91.82%) as an off-white solid.

### Synthesis of 44f

Into a 100 mL 3-necked round-bottom flask were added 44e (1.90 g, 5,333 mmol, 1.00 equiv), NaNO₂ (3.68 g, 53,335 mmol, 10 equiv), EtOAc (4.00 mL), 120 (20.00 mL) at room temperature. To the above HNO₃ (53.3 mL, 53.335 mmol, 10 equiv, 1M) was added dropwise at room temperature. The resulting mixture was stirred overnight at room temperature. The resulting mixture was extracted with EtOAc (3×100 mL). The resulting mixture was concentrated under vacuum to afford 44f (13 g, 69.17%) as an off-white solid.

### Synthesis of 44g

Into a 100 mL 3-necked round-bottom flask were added 44f (0.90 g, 5.861 mmol, 1.00 equiv) and DCM (20.00 mL) at room temperature. To the above mixture was added DAST (1889.45 mg, 11.722 mmol, 2 equiv) dropwise at 0° C. The resulting mixture was stirred for additional 1 h at 0° C. The reaction was quenched with NaHCO₃ (20 mL) at room temperature. The aqueous layer was extracted with DCM (3×20 mL). The resulting mixture was concentrated under vacuum. The residue was purified by silica gel column chromatography, eluted with PE/EtOAc (1:1) to afford 44g (1 g, 52.31%) as a yellow solid.

### Synthesis of 44h

Into a 50 mL round-bottom flask were added 44g (1.00 g, 3.066 mmol, 1.00 equiv) and CH₃CN (20.00 mL), NH₃·H₂O (20 mL), Cu₂O (43.87 mg, 0.307 mmol, 0.1 equiv) at room temperature. The resulting mixture was stirred overnight at 100° C. The reaction was diluted with water (60 mL) at room temperature. The aqueous layer was extracted with EtOAc (3×100 ml). The resulting mixture was concentrated under vacuum. The residue was purified by prep-TLC (CH₂Cl₂/MeOH 20:1) to afford 44h (680 mg, 84.56%) as a yellow solid.

### Synthesis of 44i

Into a 25 mL 3-necked round-bottom flask were added 44h (650.00 mg, 2.478 mmol, 1.00 equiv) and DCE (10.00 mL), 5-bromo-3-(trifluoromethyl)pyridine-2-carbaldehyde (1258.95 mg, 4.956 mmol, 2 equiv), NaBH(OAc)3 (1050.46 mg, 4.956 mmol, 2 equiv), HOAc (446.46 mg, 7.435 mmol, 3 equiv) at room temperature. The resulting mixture was stirred overnight at room temperature. The reaction was quenched with water (20 mL) at room temperature. The aqueous layer was extracted with DCM (3×20 mL). The resulting mixture was concentrated under vacuum. The residue was purified by prep-TLC (CH₂Cl₂/MeOH 20:1) to afford 44i (980 mg, 79.04%) as a yellow solid.

### Synthesis of 44j

Into a 25 mL 3-necked round-bottom flask were added 44i (980.00 mg, 1,959 mmol, 1.00 equiv) and DCM (10.00 mL), pyridine (309.89 mg, 3.918 mmol 2.00 equiv) at room temperature. To the above mixture was added triphosgene (232.50 mg, 0.784 mmol, 0.40 equiv) at 0° C. The resulting mixture was stirred for additional 1 h at 0° C. The reaction was quenched with NaHCO₃ (aq. 20 mL) at room temperature. The aqueous layer was extracted with DCM (3×20 mL). The resulting mixture was concentrated under vacuum. The residue was purified by prep-TLC (CH₂Cl₂/MeOH 20:1) to afford 44j (900 mg, 87.30%) as a yellow solid,

### Synthesis of 44k

Into a 50 mL pressure tank reactor were added 44j (300.00 mg, 0.570 mmol, 1.00 equiv) and dioxane (20.00 mL), Pd(OAc)2 (12.80 mg, 0.057 mmol, 0.1 equiv), TMEDA (132.48 mg, 1.140 mmol, 2 equiv), cataCXium (81.86 mg, 0.228 mmol, 0.4 equiv) at room temperature. The resulting mixture was stirred overnight at 80° C. under H₂:CO=1:1 atmosphere, 20 atm. The resulting mixture was concentrated under vacuum. The residue was purified by prep-TLC (CH₂Cl₂MeOH 20:1) to afford 44k (150 mg, 55.35%) as a yellow solid.

### Synthesis of 44l

Into a 8 mL vial were added 44k (140.00 mg, 0.294 mmol, 1.00 equiv) and DCE (2.00 mL), 4-fluoropiperidine (60.75 mg, 0,589 mmol, 2 equiv), NaBH(OAc)₃ (124.83 mg, 0,589 mmol, 2 equiv) at room temperature. The resulting mixture was stirred overnight at room temperature. The reaction was quenched with water at room temperature. The aqueous layer was extracted with EtOAc (3×10 mL). The resulting mixture was concentrated under vacuum. The residue was purified by prep-TLC (CH₂Cl₂/MeOH 15:1) to afford 441 (90 mg, 54.33%) as a yellow solid.

### Synthesis of 44

The 44l (90 mg) was purified by prep-chiral HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 50% B in 17 min; Wave Length: 220/254 nm; RT1 (min): 11.29) to afford Compound 44 (15.5 mg) as a yellow solid.

LCMS: (ES, m/z): [M+H]⁺ 563

¹H NMR: (300 MHz, DMSO-d₆, ppm): δ 1.72 (s, 2H), 1.96 (s, 2H), 2.33-2.36 (m, 2H), 2.55 (s, 2H), 3.28 (s, 5H), 4.60-4.85 (m, 1H), 4.86-4.87 (d, 1H), 5.18-5.22 (m, 2H), 5.34-5.36 (d, 1H), 6.22-6.34 (s, 1H), 7.03 (s, 1H), 7.13-7.15 (d, 1H), 7.34 (s, 1H), 7.42-7.46 (m, 1H), 7.56 (s, 1H), 7.69 (s, 1H), 7.78-7.80 (m, 1H), 8.38 (s, 1H).

### Example 45. Synthesis of Compound 45

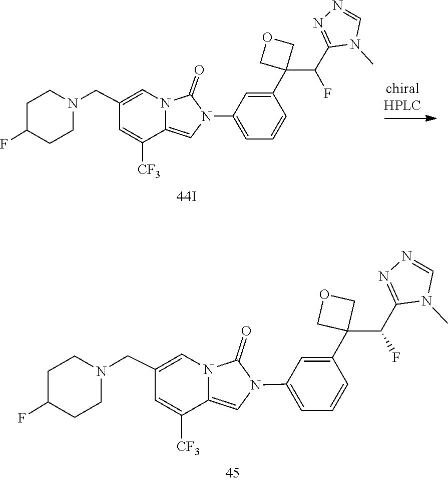 [SMILES: Cn1cnnc1C(F)C1(c2cccc(-n3cc4c(C(F)(F)F)cc(CN5CCC(F)CC5)cn4c3=O)c2)COC1 ; Cn1cnnc1[C@H](F)C1(c2cccc(-n3cc4c(C(F)(F)F)cc(CN5CCC(F)CC5)cn4c3=O)c2)COC1]

### Synthesis of 45

The 441 (90 mg) was purified by prep-chiral HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 50% B in 17 min; Wave Length: 220/254 nm; RT1 (min): 11.29) to afford Compound 45 (14.5 mg) as a yellow solid.

LCMS: (ES, m/z): [M+H]⁺ 563

¹H NMR: (400 MHz, DMSO-d₆, ppm): δ 1.72 (s, 2H), 1.96 (s, 2H), 2.33-2.36 (m, 2H), 2.55 (s, 2H), 3.28 (s, 5H), 4.60-4.85 (m, 1H), 4.86-4.87 (d, 1H), 5.18-5.22 (m, 2H), 5.34-5.36 (d, 1H), 6.22-6.34 (s, 1H), 7.03 (s, 1H), 7.13-7.15 (d, 1H), 7.34 (s, 1H), 7.42-7.46 (m, 1H), 7.56 (s, 1H), 7.69 (s, 1H), 7.78-7.80 (m, 1H), 8.38 (s, 1H).

### Example 46. Synthesis of Compound 46

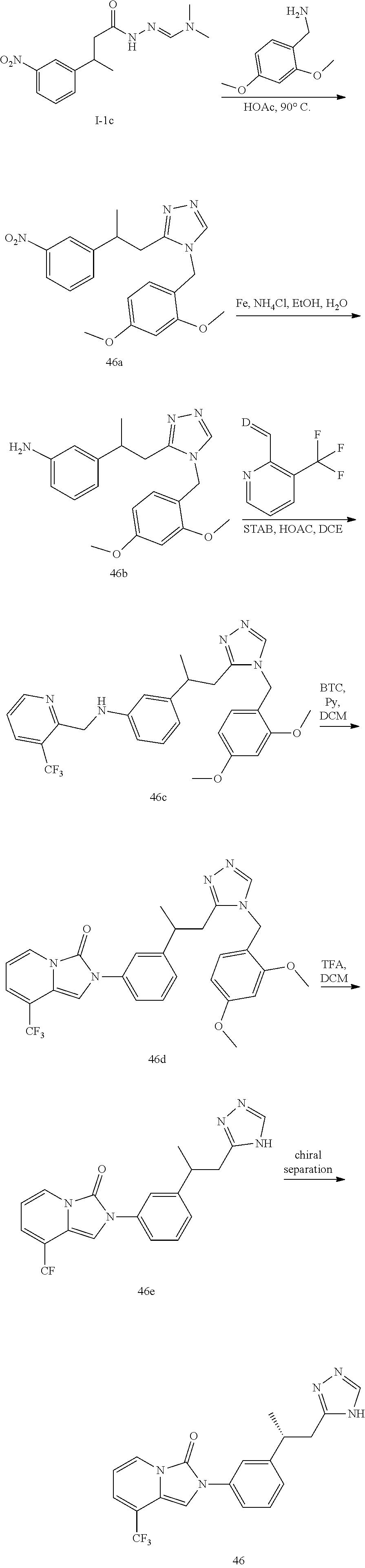

### Synthesis of 46a t.762

To a stirred solution of I-1c (3.60 g, 12.935 mmol, 1.00 equiv) in HOAc (50.00 mL) were added 1-(2,4-dimethoxyphenyl)methanamine (21.63 g, 129.360 mmol, 1000 equiv) at room temperature under nitrogen atmosphere. The resulting mixture was stirred for 3 h at 90° C. The resulting mixture was diluted with water (100 mL). The aqueous layer was extracted with EtOAc (2×100 mL). The residue was purified by silica gel column chromatography, eluted with PE/EtOAc (10:1) to afford 46a (1.2 g, 21.35%) as a yellow oil.

### Synthesis of 46b

To a stirred solution of 46a (1.20 g, 3.138 mmol, 1.00 equiv) in EtOH (50.00 mL) was added NH₄Cl (1.68 g, 31.379 mmol, 10 equiv) in H₂O (50.00 mL) at room temperature under nitrogen atmosphere. The resulting mixture was stirred for overnight at 80° C. under nitrogen atmosphere. The resulting mixture was diluted with water (100 mL). The aqueous layer was extracted with CH₂Cl₂/MeOH=10:1 (3×100 mL). The residue was purified by prep-TLC (CH₂Cl₂/MeOH 20:1) to afford 46b (11 g, 90.51%) as a light yellow oil.

### Synthesis of 46c

To a stirred solution of 46b (1.10 g, 3.121 mmol, 1.00 equiv) and 3-(trifluoromethyl)pyridine-2-carbaldehyde (0.55 g, 0.003 mmol, 1 equiv) in DCE (50.00 mL) were added HOAc (0.37 g, 0.006 mmol, 2 equiv) and STAB (1.98 g, 0.009 mmol, 3 equiv) at room temperature under nitrogen atmosphere. The resulting mixture was stirred for 6h at room temperature. The resulting mixture was washed with water (50 mL). The residue was purified by prep-TLC (CH₂Cl₂/MeOH 20:1) to afford 46c (1.4 g, 78.92%) as a yellow oil.

### Synthesis of 46d

To a stirred solution of 46c (1.50 g 2.932 mmol, 1.00 equiv) and pyridine (1.39 g 17.594 mmol, 6 equiv) in DCM (60.00 mL) was added triphosgene (0.30 g, 1.026 mmol, 0.35 equiv) at 0° C. under nitrogen atmosphere. The resulting mixture was stirred for 10 min at room temperature. The resulting mixture was washed NaHCO₃ (aq.) (100 mL). The residue was purified by prep-TLC (CH₂Cl₂/MeOH 20:1) to afford 46d (1.2 g, 75.86%) as a yellow solid.

### Synthesis of 46e

To a stirred solution of 46d (1.20 g, 2,232 mmol, 1.00 equiv) in DCM (10.00 mL) was added TFA (10.00 mL) at 0° C. under nitrogen atmosphere. The resulting mixture was stirred overnight at room temperature. The resulting mixture was concentrated under reduced pressure. The residue was purified by prep-TLC (CH₂Cl₂/MeOH 20:1) to afford 46e (300 mg, 32.96%) as a yellow solid,

### Synthesis of 46

The crude product (46e, 300 mg) was purified by prep-Chiral HPLC with the following conditions (Column: CHIRALPAK IC, 2*25 cm, 5 μm; Mobile Phase A: MtBE (0.5% 2M NH₃-MeOH), Mobile Phase B: EtOH:DCM=1:1; Flow rate: 20 mL/min; Gradient: 15% B to 15% B in 12.5 min; Wave Length: 254 un; RT1 (min): 9.43) to afford Compound 46 (90.6 mg, 29.90%) as a yellow solid.

LCMS: (ES, m/z): [M+H]⁺ 388

¹H NMR: (400 MHz, CD₃OD-d4, ppm): δ 1.27-1.39 (d, 3H), 3.03-3.15 (m, 2H), 3.37-3.42 (m, 1H), 6.34-6.38 (m, 1H), 7.04-7.05 (d, 1H), 7.11 (s, 1H), 7.18-7.29 (m, 1H), 7.41-7.45 (m, 1H), 7.54-7.56 (m, 1H), 7.56-7.60 (m, 1H), 7.77-7.79 (m, 1H), 7.79-8.40 (m, 1H).

### Example 47. Synthesis of Compound 47

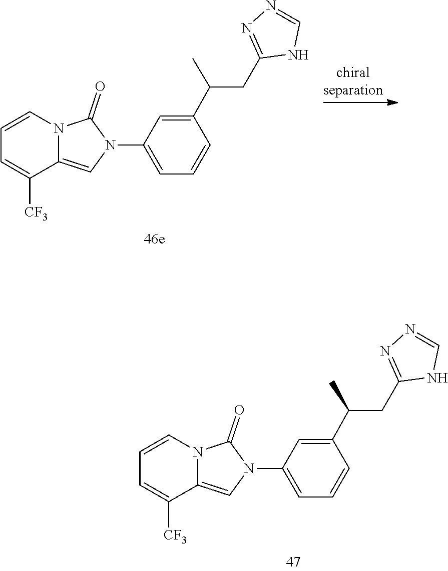 [SMILES: CC(Cc1nnc[nH]1)c1cccc(-n2cc3c(C(F)(F)F)cccn3c2=O)c1 ; C[C@@H](Cc1nnc[nH]1)c1cccc(-n2cc3c(C(F)(F)F)cccn3c2=O)c1]

### Synthesis of 47

Compound 46e (300 mg) was purified by prep-Chiral HPLC with the following conditions (Column: CHIRALPAK IC, 2*25 cm, 5; Mobile Phase A: MtBE (0.5% 2M NH₃-MeOH), Mobile Phase B: EtOH:DCM=1:1; Flow rate: 20 mL/min; Gradient: 15% B to 15% B in 12.5 min; Wave Length: 220/254 nm; RT2 (min): 10.8) to afford Compound 47 (95.0 mg, 31.35%) as a yellow solid.

LCMS: (ES, m/z): [M+H]⁺ 388

¹H NMR: (400 MHz, CD₃OD-d4, ppm): δ 1.30-1.43 (d, 3H), 3.03-3.15 (m, 2H), 3.37-3.42 (m, 1H), 6.34-6.38 (m, 1H), 7.03-7.05 (d, 1H), 7.11 (s, 1H), 7.18-7.25 (m, 1H), 7.41-7.45 (m, 1H), 7.53-7.55 (m, 1H), 7.55-7.59 (m, 1H), 7.76-7.78 (m, 1H), 7.88-8.40 (m, 1H).

### Example 48. Synthesis of Compound 48

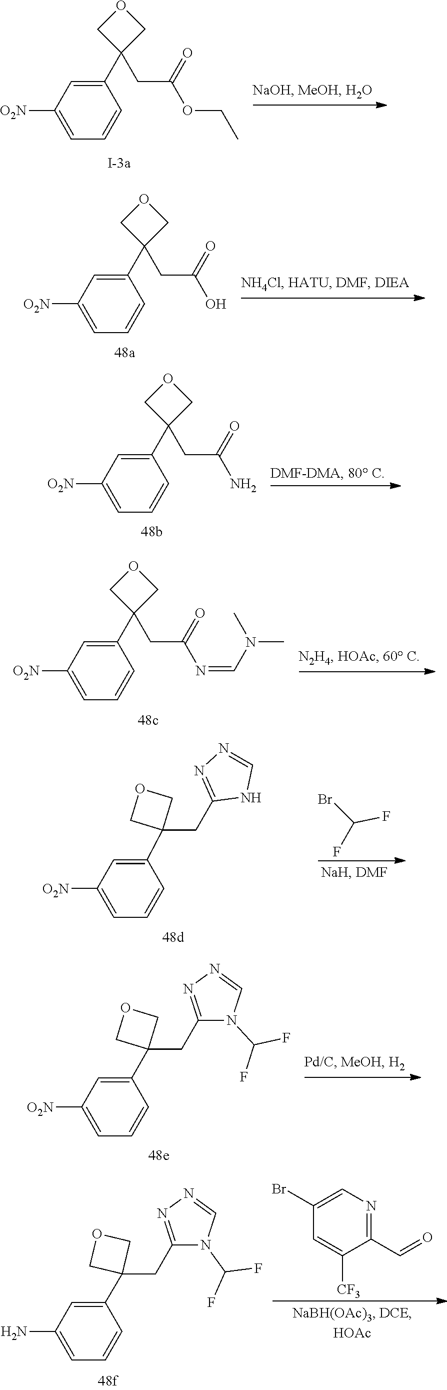

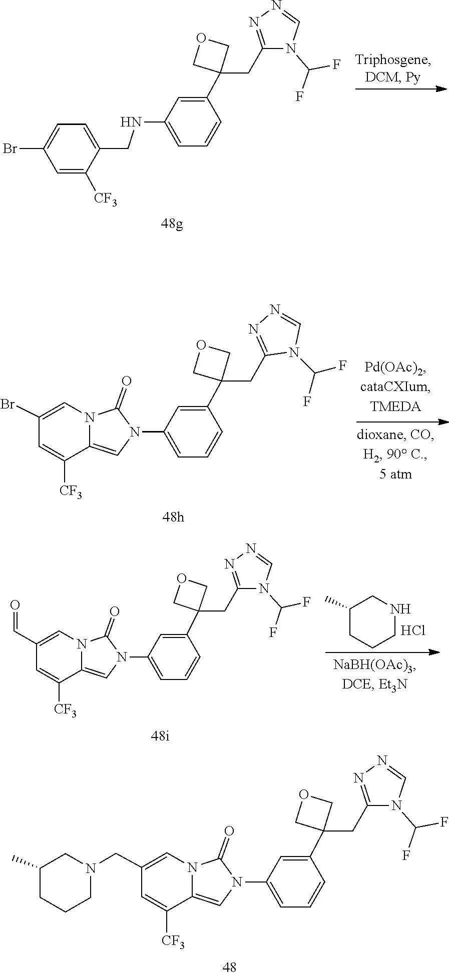

### Synthesis of 48a

To a stirred solution of I-3a (5.00 g, 18,867 mmol, 1.00 equiv) in MeOH (100.00 mL) was added NaOH (29.85 mL, 29,850 mmol, 1.50 equiv, 1M). The resulting mixture was stirred overnight at room temperature. The MeOH was removed under vacuum. The mixture was acidified to pH 4 with HCl (1 M). The precipitated solids were collected by filtration and washed with water (10 mL). The resulting solid was dried in an oven under reduced pressure. This resulted in 48a (4 g, 89.45%) as a white solid,

### Synthesis of 48b

To a stirred mixture of 48a (4.00 g, 16.863 mmol, 1.00 equiv) and NH₄Cl (2.71 g, 50.589 mmol, 3.00 equiv) in DMF (100.00 mL) were added DIEA (8.72 g, 67.470 mmol, 4.00 equiv) and HATU (9.62 g, 25.295 mmol, 1.50 equiv). The resulting mixture was stirred overnight at room temperature under argon atmosphere. The resulting mixture was diluted with water (300 mL). The aqueous layer was extracted with EtOAc (3×150 mL). The resulting mixture was concentrated under vacuum. The residue was purified by trituration with water (15 mL). The resulting solid was dried in an oven under reduced pressure. This resulted in 48b (3.2 g, 80.33%) as a light brown solid.

### Synthesis of 48c

A solution of 48b (3.50 g, 1 equiv) in DMF-DMA (16.00 mL) was stirred overnight at 80° C. The resulting mixture was diluted with water (50 mL). The resulting mixture was extracted with EtOAc (3×50 mL). The combined organic layers were washed with brine (15 mL), dried over anhydrous Na₂SO₄. After filtration, the filtrate was concentrated under reduced pressure. This resulted in 48c (3.4 g, 78.78%) as a grey solid.

### Synthesis of 48d

To a stirred solution of 48c (3.20 g, 10.985 mmol, 100 equiv) in HOAc (16.00 mL) was added hydrazine hydrate (14.00 mL, 98%) dropwise at room temperature. The resulting mixture was stirred overnight at 60° C. The resulting mixture was diluted with water (200 mL). The aqueous layer was extracted with EtOAc (3×100 mL). The combined organic layers were dried over anhydrous Na₂SO₄. After filtration, the filtrate was concentrated under reduced pressure. This resulted in 48d (2.9 g, 101.44%) as a grey solid.

### Synthesis of 48e

To a stirred solution of 48d (2.90 g, 11.143 mmol, 1.00 equiv) in DMF (30.00 mL) was added NaH (2.23 g, 55,715 mmol, 5.00 equiv, 60%) in portions at 0° C. under argon atmosphere. The resulting mixture was stirred for 30 min at 0° C. under argon atmosphere. To the above mixture was added bromodifluoromethane (87.53 g, 66,858 mmol, 6.00 equiv, 10% in DMF). The resulting mixture was stirred overnight at room temperature. The reaction was quenched by the addition of water (400 mL). The aqueous layer was extracted with EtOAc (3×150 mL) The resulting mixture was concentrated under vacuum. The residue was purified by reverse flash chromatography with the following conditions: column, C18 silica gel; mobile phase, MeCN in water (10 mmol NH₃H₂O), 0% to 80% gradient in 35 min; detector, UV 254 nm. This resulted in 48e (0.8 g, 23.14%) as a white solid.

### Synthesis of 48f

To a solution of 48e (780.00 mg, 2.514 mmol, 1.00 equiv) in MeOH (30.00 mL) was added Pd/C (70.00 mg) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. This resulted in 48f (710 mg, 92.70%) as a light brown solid.

### Synthesis of 48g

To a stirred solution of 48f (300.00 mg, 1.070 mmol, 1.00 equiv) and 5-bromo-3-(trifluoromethyl)pyridine-2-carbaldehyde (271.88 mg, 1.070 mmol, 1.00 equiv) in DCE (10.00 mL) was added HOAc (64.28 mg, 1.070 mmol, 1 equiv) and NaBH(OAc)₃ (680.56 mg, 3.211 mmol, 3 equiv) at room temperature under nitrogen atmosphere. The resulting mixture was stirred overnight at room temperature. The resulting mixture was washed with 10 mL of water. The residue was purified by prep-TLC (CH₂Cl₂/MeOH 15:1) to afford 48g (370 mg, 63.48%) as a light yellow solid.

### Synthesis of 48h

To a stirred solution of 48g (350.00 mg, 0.677 mmol, 1.00 equiv) and pyridine (321.11 mg 4.060 mmol, 6 equiv) in DCM (10.00 mL) was added triphosgene (70.27 mg, 0.237 mmol, 0.35 equiv) at 0° C. under nitrogen atmosphere. The resulting mixture was stirred for 30 min at room temperature. The resulting mixture was washed with 10 mL of water. The residue was purified by prep-TLC (CH₂Cl₂/MeOH 20:1) to afford 48h (300 mg, 79.02%) as a yellow solid.

### Synthesis of 48i

To a solution of 48h (280.00 mg, 0.514 mmol, 1.00 equiv) in dioxane (8.00 mL) was added cataCXium (36.89 mg, 0.103 mmol, 0.20 equiv), Pd(OAc)₂. (11.55 mg, 0.051 mmol, 0.1 equiv), TMEDA (119.56 mg, 1.029 mmol, 2 equiv) in a pressure tank. The mixture was purged with nitrogen for 3 min and then was pressurized to 10 atm with CO:H₂=1:1 at 90° C. for overnight. The reaction mixture was cooled to room temperature and diluted with water. The aqueous layer was extracted with EtOAc (2×50 mL). The residue was purified by prep-TLC (CH₂Cl₂/MeOH 20:1) to afford 48i (160 mg, 57.36%) as a yellow solid,

### Synthesis of 48

To a stirred solution of 48i (150.00 mg, 0.304 mmol, 1.00 equiv) and (3S)-3-methylpiperidine hydrochloride (123.71 mg, 0.912 mmol, 3.00 equiv) in DCE (6.00 mL) was added Et₃N (92.29 mg, 0,912 mmol, 3 equiv) and NaBH(OAc)₃ (19330 mg, 0.912 mmol, 3 equiv) at room temperature under nitrogen atmosphere. The resulting mixture was stirred overnight at room temperature. The resulting mixture was diluted with CH₂Cl₂ (20 mL). The resulting mixture was washed with 20 mL of water. The residue was purified by prep-TLC (CH₂Cl₂/MeOH 20:1) to afford Compound 48 (60.0 mg, 33.89%) as a yellow solid,

LCMS: (ES, m/z): [M+H]⁺ 577

¹H NMR: (400 MHz, DMSO-d6, ppm): δ 0.82-0.86 (d, 3H), 1.41-1.50 (m, 1H), 1.50-1.66 (m, 4H), 1.91-1.94 (m, 2H), 2.74-2.84 (m, 2H), 3.27 (s, 2H), 3.75 (s, 2H), 4.95 (s, 4H), 7.01-7.02 (m, 1H), 7.06-7.08 (m, 1H), 7.31 (s, 1H), 7.38-7.55 (m, 3H), 7.70-7.72 (m, 1H), 7.73-7.75 (d, 1H), 8.82 (s, 1H).

### Example 49. Synthesis of Compound 49

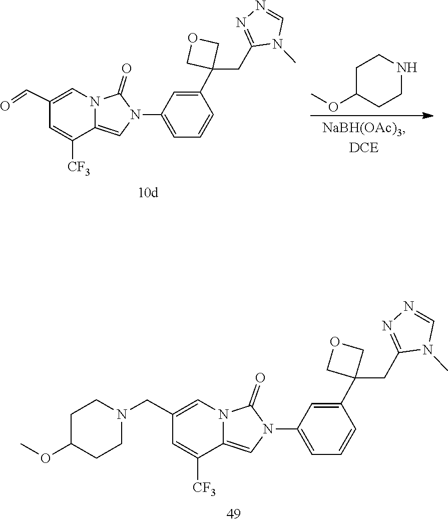

### Synthesis of 49

To a stirred solution/mixture of 10d (100 mg, 0.21 mmol, 1.0 equiv) and 4-methoxypiperidine (25 mg, 0.21 mmol, 1.0 equiv) in DCE (1 mL) was added NaBH(OAc)3 (923 mg, 0.43 mmol, 2.0 equiv) in portions at room temperature under nitrogen atmosphere. The resulting mixture was stirred overnight at room temperature under nitrogen atmosphere. The reaction was quenched with NH₄Cl (aq.) (20 mL) at room temperature. The resulting mixture was extracted with CH₂Cl₂ (3×10 mL). The combined organic layers were concentrated under reduced pressure. The crude product (100 mg) was purified by prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 27% B to 42% B in 8 min, 42% B; Wave Length: 254/220 nm; RT1 (min): 7.38) to afford Compound 49 (18.8 mg, 15%) as a yellow solid.

LCMS: (ES, m/z): [M+H]⁺ 557

¹H NMR: (400 MHz, DMSO-d6, δ ppm): δ 1.42-1.44 (m, 2H), 1.81-1.83 (m, 2H), 2.11-2.16 (m, 2H), 2.61-2.67 (m, 2H), 2.97 (s, 3H), 3.18-3.22 (m, 4H), 3.28-3.30 (m, 2H), 3.53 (s, 2H), 4.91-4.96 (m, 4H), 6.88-6.90 (d, 1H), 7.01 (s, 1H), 7.31 (s, 1H), 7.38-7.42 (m, 2H), 7.66 (s, 1H), 7.74-7.76 (d, 1H), 8.20 (s, 1H).

### Example 50. Synthesis of Compound 50

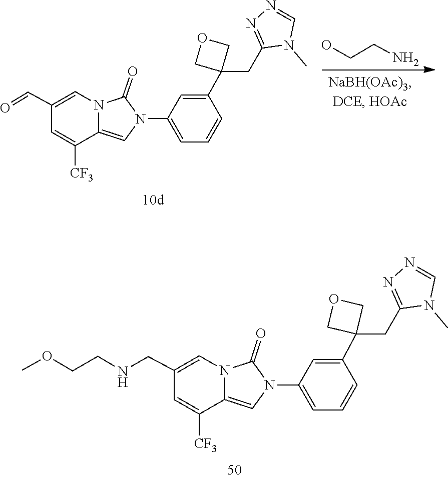

### Synthesis of 50

Into a 8 mL sealed tube were added 10d (100.00 mg, 0.219 mmol, 1.00 equiv) and DCE (1.00 mL) and 2-methoxy-ethanamine (16.42 mg, 0.219 mmol, 1 equiv), NaBH(OAc)₃ (92.67 mg, 0,437 mmol, 2 equiv) and HOAc (26.26 mg, 0.437 mmol, 2 equiv) at room temperature. The resulting mixture was stirred overnight at room temperature. The reaction was quenched by the addition of sat. NH₄Cl (aq.) (20 mL) at room temperature. The aqueous layer was extracted with CH₂Cl₂ (3×30 mL). The resulting mixture was concentrated under vacuum. The crude product (100 mg) was purified by prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 15% B to 45% B in 8 min, 45% B; Wave Length: 254 nm; RT1 (min): 7.23 to afford Compound 50 (30.9 mg, 27.04%) as a yellow solid,

LCMS: (ES, m/z): [M+H]⁺ 517

¹H NMR: (400 MHz, DMSO-d₆, ppm): δ 2.62-2.64 (d, 2H), 3.22 (s, 3H), 3.33-3.38 (m, 3H), 3.40-3.43 (m, 2H), 3.52-3.53 (d, 4H), 4.91-4.96 (m, 4H), 6.87-6.89 (d, 1H), 7.12 (s, 1H), 7.30 (s, 1H), 7.38-7.41 (m, 2H), 7.42 (s, 1H), 7.68-7.77 (d, 1H), 8.20 (s, 1H).

Alternatively, Compound 50 may be also prepared in the manner outlined below:

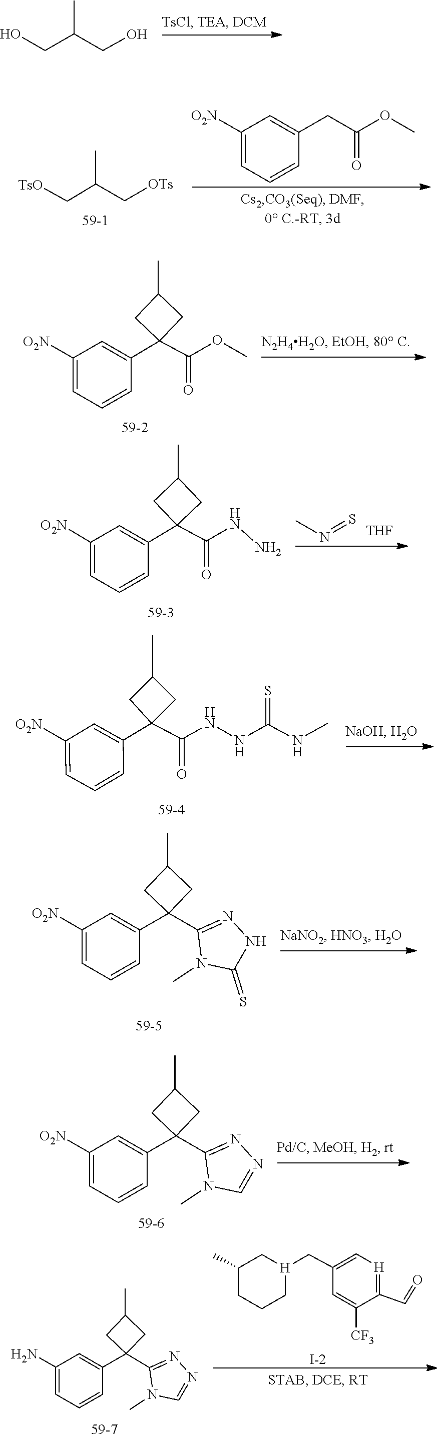

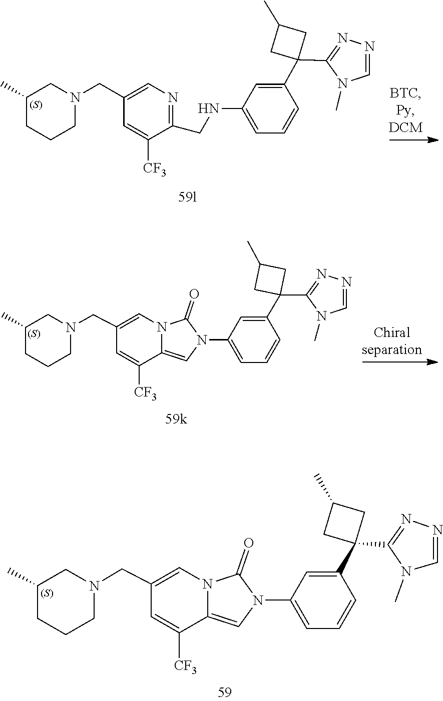

### 1. Synthesis of 59-1

To a solution of methylpropanediol (59 g, 554.803 mmol, 1 equiv) and TsCl (264.42 g, 1387.007 mmol, 2.5 equiv) in DCM (590 mL) was added TEA (168.43 g, 1664.409 mmol, 3 equiv) dropwise at room temperature. The mixture was stirred for 12h at room temperature under nitrogen atmosphere. The reaction was quenched with water (800 mL) at room temperature. The aqueous layer was extracted with DCM (2×800 mL). The resulting mixture was concentrated under reduced pressure. The residue was purified by silica gel column chromatography, eluted with DCM/MeOH (80:1) to afford 59-1 (190 g, 79.38%) as a yellow oil.

### 2. Synthesis of 59-2

To a stirred solution of methyl 2-(3-nitrophenyl)acetate (59 g, 256.182 mmol, 1.00 equiv) and Cs₂CO₃ (392.30 g, 1204.055 mmol, 5 equiv) in DMF (590 mL) was added 59-1 (190 g, 476.800 mmol, 1.86 equiv) at room temperature. The resulting mixture was stirred for 3 days at room temperature under nitrogen atmosphere. The reaction was quenched with NH₄Cl (aq.) at room temperature. The aqueous layer was extracted with EtOAc (3×600 mL). The resulting mixture was concentrated under reduced pressure. The residue was purified by silica gel column chromatography, eluted with PE/EA (80:1) to afford 59-2 (18 g, 26.99%) as a yellow oil.

### 3. Synthesis of 59-3

A solution of 59-2 (51 g, 204.601 mmol, 1 equiv) and hydrazine hydrate (65.56 g, 2046.010 mmol, 10 equiv) in EtOH (510 mL) was stirred overnight at 80° C. The reaction was quenched by the addition of NH₄Cl (aq.) (800 mL) at room temperature. The precipitated solids were collected by filtration and washed with water (3×100 mL). This resulted in 59-3 (46 g, 81.18%) as an off-white solid.

### 4. Synthesis of 59-4

A solution of 59-3 (46 g, 184.539 mmol, 1 equiv) and methyl isothiocyanate (26.98 g, 369.078 mmol, 2 equiv) in THF (460 mL) was stirred for 5h at room temperature. The reaction was quenched with water (400 mL) at room temperature. The resulting mixture was concentrated under reduced pressure. The precipitated solids were collected by filtration and washed with water (3×100 mL). This resulted in 59-4 (45 g, 68.08%) as a yellow solid.

### 5. Synthesis of 59-5

To a stirred solution of 59-4 (45 g, 139.587 mmol, 1 equiv) in H₂O (459 mL) was added NaOH (55.83 g, 1395.870 mmol, 10 equiv) at room temperature. The resulting mixture was stirred for 5h at room temperature. The mixture was acidified to pH 5 with HCl (aq.). The precipitated solids were collected by filtration and washed with water (3×200 mL). This resulted in 59-5 (42 g, 88.97%) as a yellow solid.

### 6. Synthesis of 59-6

To a stirred solution of 59-5 (42 g, 137.990 mmol, 1 equiv) and NaNO₂ (95.21 g, 1379.900 mmol, 10 equiv) in H₂O (420 mL) was added HNO₃ (1380 mL, 1379.90 mmol, 10 equiv, 1M) dropwise at room temperature. The resulting mixture was stirred for 5h at room temperature. The mixture was neutralized with saturated NaHCO₃ (aq.) (590 mL). The aqueous layer was extracted with EtOAc (3×400 mL). The resulting mixture was concentrated under reduced pressure. The residue was purified by silica gel column chromatography, eluted with DCM/MeOH (80:1) to 59-6 (30 g, 71.85%) as a yellow solid.

### 7. Synthesis of 59-7

To a solution of 59-6 (30 g, 110.169 mmol, 1 equiv) in MeOH (590 mL) was added Pd/C (10%, 3.0 g) under nitrogen atmosphere. The mixture was hydrogenated at room temperature for overnight under hydrogen atmosphere using a hydrogen balloon, filtered through a Celite pad and concentrated under reduced pressure. This resulted in 59-7 (22 g, 74.17%) as a yellow solid.

### 8. Synthesis of 59l

To a stirred solution of 59-7 (10 g, 41.267 mmol, 1 equiv) and I-2 (14.18 g, 49.520 mmol, 1.2 equiv) in DCE (100 mL) was added STAB (17.49 g, 82.534 mmol, 2 equiv) at room temperature. The resulting mixture was stirred overnight at room temperature. The reaction was quenched with saturated Na₂CO₃ (aq.) (159 mL) at room temperature. The aqueous layer was extracted with DCM (3×159 mL). The resulting mixture was concentrated under reduced pressure. The residue was purified by silica gel column chromatography, eluted with DCM/MeOH (70:1) to 591 (16.7 g, 71.05%) as a yellow solid.

### 9. Synthesis of 59k

To a stirred solution of 59l (16.5 g, 32.187 mmol, 1 equiv) and Pyridine (15.28 g, 193.122 mmol, 6 equiv) in DCM (200 mL) was added triphosgene (3.53 g, 11.909 mmol, 0.37 equiv) at 0° C. The resulting mixture was stirred for 10 min at room temperature. The reaction was quenched with saturated NaHCO₃ (aq.) (159 mL) at room temperature. The aqueous layer was extracted with DCM (3×159 mL). The resulting mixture was concentrated under reduced pressure. The residue was purified by reverse flash chromatography with the following conditions: column, C18 silica gel; mobile phase, MeCN in Water (10 mmol/L NH₄HCO₃), 30% to 59% gradient in 30 min; detector, UV 254 nm. This resulted in 59k (12 g, 65.76%) as a yellow solid.

### 10. Synthesis of 50

The 59k (11.5 g) was purified by Prep-SFC with the following conditions (Column: CHIRALPAK AD-H, 5*25 cm, 5 μm; Mobile Phase A: CO₂, Mobile Phase B: ETOH (0.1% 2M NH₃-MEOH); Flow rate: 200 mL/min; Gradient: isocratic 40% B; Column Temperature (° C.): 35; Back Pressure (bar): 100; Wave Length: 220 nm; RT1 (min): 3.55; RT2 (min): 4.71; the second peak is product) to 50 (2.0630 g, 19.11%) as a yellow solid. LC-MS-50 (ES, m/z): [M+H]⁺ 539. H-NMR-59: (400 MHz, DMSO-d6, δ ppm): 0.79-0.84 (d, 3H), 0.86-0.92 (m, 1H), 1.01-1.18 (d, 3H), 1.41-1.52 (m, 1H), 1.53-1.65 (m, 4H), 1.91-2.03 (m, 1H), 2.25-2.40 (m, 3H), 2.69-2.77 (m, 2H), 3.07-3.10 (m, 2H), 3.25 (s, 3H), 3.34-3.42 (m, 2H), 7.02 (s, 1H), 7.08-7.10 (d, 1H), 7.35 (s, 1H), 7.44-7.48 (m, 1H), 7.61-7.63 (d, 1H), 7.67-7.71 (m, 2H), 8.37 (s, 1H).

### Example 51. Synthesis of Compound 51

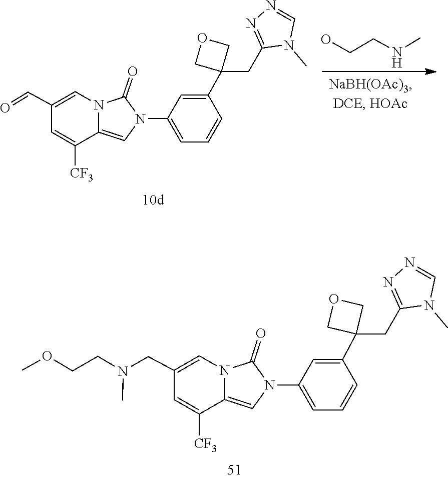

### Synthesis of 51

Into a 8 mL sealed tube were added 10d (100.00 mg, 0.219 mmol, 1.00 equiv) and (2-methoxyethyl)(methyl)amine (19.49 mg, 0.000 mmol, 1.00 equiv), NaBH(OAc)₃ (92.67 mg, 0.438 mmol, 2.00 equiv), HOAc (26.26 mg, 0.438 mmol, 2.00 equiv) and DCE (1.50 mL). The resulting mixture was stirred for 2h at room temperature. The reaction was quenched by the addition of sat. NH₄Cl (aq.) (20 mL) at room temperature. The aqueous layer was extracted with CH₂Cl₂ (3×30 mL). The resulting mixture was concentrated under vacuum. The crude product (80 mg) was purified by prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 20% B to 50% B in 8 min, 50% B; Wave Length: 220/254 nm; RT1 (min): 7.23 to afford Compound 51 (34.9 mg, 29.85%) as a yellow solid.

LCMS: (ES, m/z): [M+H]⁺ 531

¹H NMR: (400 MHz, DMSO-d₆, ppm): δ 2.30 (s, 2H), 2.50-2.57 (m, 2H), 2.93-2.97 (m, 3H), 3.32 (s, 3H), 3.40-3.46 (m, 2H), 3.53 (s, 2H), 3.60 (s, 2H), 4.88-4.96 (m, 4H), 6.87-6.89 (d, 1H), 7.01 (s, 1H), 7.31 (s, 1H), 7.38-7.42 (m, 2H), 7.68-7.77 (m, 1H), 8.20 (s, 1H).

### Example 52. Synthesis of Compound 52

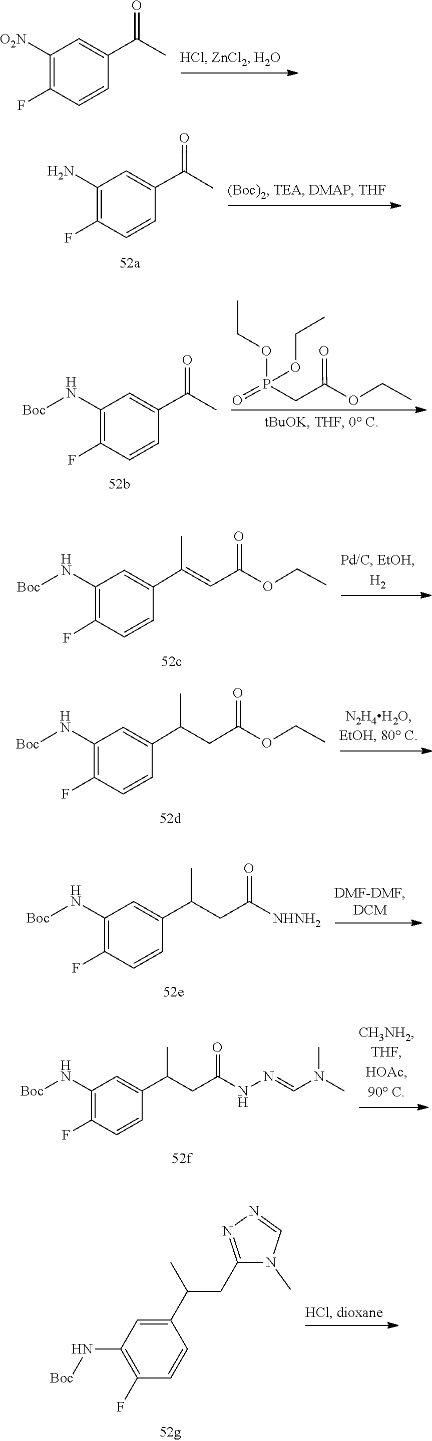

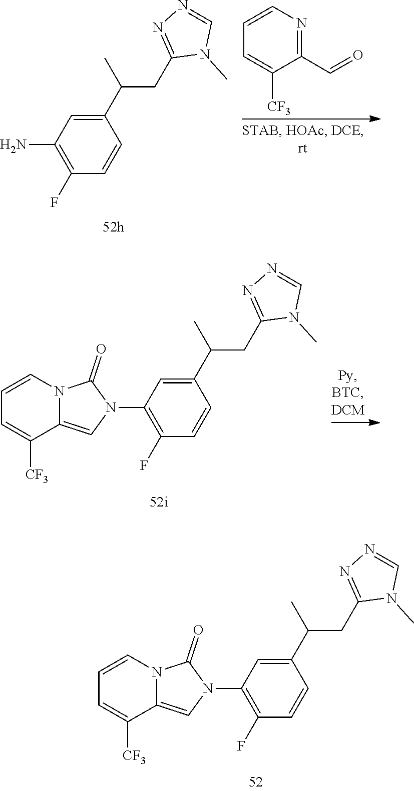

### Synthesis of 52a

To a stirred solution of 1-(4-fluoro-3-nitrophenyl)ethanone (30.00 g, 163.811 mmol, 1.00 equiv) in HCl (78.00 mL, 1M) was added SnCl₂ (93.19 g, 491.457 mmol, 3.00 equiv), H₂O (300.00 mL) dropwise at 0° C. under nitrogen atmosphere. The resulting mixture was stirred for 15 min at 0° C. under nitrogen atmosphere. The resulting mixture was stirred for 25 min at 100° C. under nitrogen atmosphere. The resulting mixture was stirred for 2 h at room temperature under nitrogen atmosphere. The reaction was quenched with ice at room temperature. The mixture was acidified to pH 12 with NaOH. The resulting mixture was extracted with EtOAc (3×150 mL). The combined organic layers were dried over anhydrous Na₂SO₄. After filtration, the filtrate was concentrated under reduced pressure. This resulted in 52a (20 g 73.34%) as a yellow solid.

### Synthesis of 52b

To a stirred solution of 52a (5.00 g, 32.646 mmol, 1.00 equiv) and TEA (9.91 g, 97.939 mmol, 3 equiv) in THF (100.00 mL) was added DMAP (0.40 g, 3.265 mmol, 0.1 equiv) and Boc₂O (14.25 g, 65.293 mmol, 2 equiv) at room temperature under nitrogen atmosphere. The resulting mixture was stirred overnight at room temperature under nitrogen atmosphere. The resulting mixture was diluted with EtOAc (100 ML). The resulting mixture was washed with 200 mL of brine. The residue was purified by silica gel column chromatography, eluted with PE/EtOAc (30:1) to afford 52b (6 g, 65.31%) as a white solid,

### Synthesis of 52c

To a stirred solution of triethyl phosphonoacetate (7.97 g, 0.036 mmol, 3 equiv) in THF (50.00 mL) was added tert-butoxypotassium (3.99 g, 0.036 mmol, 3 equiv) at room temperature under nitrogen atmosphere. The resulting mixture was stirred for 2h at room temperature. To the above mixture was added 52b (3.00 g, 11.845 mmol, 1.00 equiv) at room temperature. The resulting mixture was stirred overnight at room temperature. The reaction was quenched with NH₄Cl (aq.) (100 mL) at room temperature. The aqueous layer was extracted with EtOAc (2×100 ml). The residue was purified by silica gel column chromatography, eluted with PE/EtOAc (15:1) to afford 52c (3 g, 70.49%) as an off-white solid.

### Synthesis of 52d

To a solution of 52c (3.00 g, 9.277 mmol, 1.00 equiv) in EtOH (100.00 mL) was added Pd/C (0.3 g) in a 500 mL round-bottom flask. The mixture was hydrogenated at room temperature overnight under hydrogen atmosphere using a hydrogen balloon, filtered through a Celite pad and concentrated under reduced pressure. The crude product was used in the next step directly without further purification. This resulted in 52d (2.8 g, 83.48%) as a light yellow oil.

### Synthesis of 52e

To a stirred solution of 52d (2.80 g, 8.605 mmol, 1.00 equiv) in EtOH (80.00 mL) was added hydrazine hydrate (98%) (4.31 g, 86.050 mmol, 10.00 equiv) at room temperature under nitrogen atmosphere. The resulting mixture was stirred for 2 days at 80° C. under nitrogen atmosphere. The resulting mixture was diluted with water (100 mL). The aqueous layer was extracted with DCM/MeOH=10:1 (3×100 mL). The residue was purified by prep-TLC (CH₂Cl₂/MeOH 25:1) to afford 52e (1.5 g, 51.51%) as an off-white solid.

### Synthesis of 52f

To a stirred solution of 52e (1.00 g, 3.212 mmol, 1.00 equiv) in DCM (10.00 mL) was added DMF-DMA (1.91 g, 16.059 mmol, 5 equiv) at room temperature under nitrogen atmosphere. The resulting mixture was stirred for 3h at room temperature. The residue was purified by prep-TLC (CH₂CO₂/MeOH 12:1) to afford 52f (800 mg, 61.18%) as an off-white solid.

### Synthesis of 52g

To a stirred solution of 52f (750.00 mg, 2.047 mmol, 100 equiv) in HOAc (3.00 mL) was added CH₃NH₂ in THF (5.00 mL, 1M) at room temperature under nitrogen atmosphere. The resulting mixture was stirred overnight at 90° C. The resulting mixture was diluted with water (50 mL). The aqueous layer was extracted with CH₂Cl₂ (3×50 mL). The residue was purified by prep-TLC (CH₂Cl₂/MeOH 12:1) to afford 52g (350 mg, 30.68%) as an off-white solid.

### Synthesis of 52h

A solution of 52g (350.00 mg, 0.628 mmol, 1.00 equiv, 60%) in HCl in 1,4-dioxane (5.00 mL) was stirred overnight. The resulting mixture was concentrated under vacuum. The residue was dissolved in DCM (5 mL). The residue was basified to pH 8 with NH₃ in MeOH. The residue was purified by prep-TLC (CH₂Cl₂/MeOH 15:1) to afford 52h (200 mg, 108.75%) as an off-white solid.

### Synthesis of 52i

To a stirred solution of 52h (180.00 mg, 0,768 mmol, 1.00 equiv) and 3-(trifluoromethyl)pyridine-2-carbaldehyde (134.54 mg, 0.768 mmol, 1.00 equiv) in DCE (10.00 mL) were added HOAc (46.14 mg, 0.768 mmol, 1 equiv) and STAB (488.51 mg, 2.305 mmol, 3 equiv) at room temperature under nitrogen atmosphere. The resulting mixture was stirred overnight at room temperature. The resulting mixture was diluted with DCM (50 mL). The resulting mixture was washed with 50 mL of water. The residue was purified by prep-TLC (CH₂Cl₂/MeOH 20:1) to afford 521 (200 mg, 60.22%) as a light yellow oil.

### Synthesis of 52

To a stirred solution of 52i (180.00 mg, 0.458 mmol, 1.00 equiv) and pyridine (217.16 mg, 2.745 mmol, 6 equiv) in DCM (20.00 m L) were added triphosgene (47.52 mg, 0.160 mmol, 0.35 equiv) at room temperature under nitrogen atmosphere. The resulting mixture was concentrated under reduced pressure. The residue was purified by prep-TIC (CH₂C12/MeOH 20:1) to afford the crude product. The crude product was purified by reverse flash chromatography with the following conditions: column, C18 silica gel; mobile phase, A: water, B: MeCN, 30% B to 40% B gradient in 15 min; detector, UV 254 nm. This resulted in Compound 52 (18.9 mg, 9.63%) as a yellow solid.

LCMS: (ES, m/z): [M+H]⁺ 420

¹H NMR: (400 MHz, CD₃OD-d4, ppm): δ 1.42-1.43 (d, 3H), 3.06-3.18 (m, 2H), 3.35-3.47 (m, 1H), 3.53 (s, 3H), 6.35-6.38 (m, 1H), 7.00 (s, 1H), 7.04-7.06 (d, 1H), 7.25-7.30 (m, 1H), 7.34-7.38 (m, 1H), 7.48-7.51 (d, 1H), 7.75-7.77 (d, 1H), 8.30 (s, 1H).

### Example 53. Synthesis of Compound 53

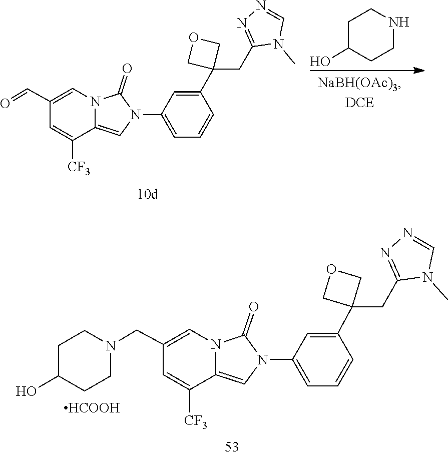

### Synthesis of 53

To a stirred solution of 10d (40 mg, 0.08 mmol, 1.0 equiv) and piperidin-4-ol (9 mg, 0.08 mmol, 1.0 equiv) in DCE (0.8 mL) was added NaBH(OAc)₃ (37 mg, 0.17 mmol, 2.0 equiv) in portions at room temperature under nitrogen atmosphere. The resulting mixture was stirred overnight at room temperature under nitrogen atmosphere. The resulting mixture was diluted with MeOH (3 mL). The resulting mixture was concentrated under reduced pressure. The crude product (50 mg) was purified by prep-HPLC with the following conditions (Column: Xselect CSH OBD Column 30*150 mm 5 μm, n; Mobile Phase A: Water (0.1% FA), Mobile Phase B: ACN; Flow rate: 60 mL/min; Gradient: 8% B to 15% B in 7 min, 15% B; Wave Length: 220 nm; RT1 (min): 7.62) to afford Compound 53 (14.5 mg, 28%) as a yellow solid.

LCMS: (ES, m/z): [M+H]⁺ 543

¹H NMR: (400 MHz, CD₃OD, δ ppm): δ 1.65-7.74 (m, 2H), 1.87-1.98 (m, 2H), 2.75-2.83 (m, 2H), 2.97 (s, 3H), 3.10-3.15 (m, 2H), 3.68 (s, 2H), 3.77-3.83 (m, 3H), 5.09 (s, 4H), 6.92-6.94 (d, 1H), 7.14-7.17 (d, 2H), 7.31 (s, 1H), 7.46-7.50 (m, 1H), 7.63-7.65 (m, 1H), 7.85 (s, 1H), 8.21-8.28 (m, 1H), 8.40 (s, 1H).

### Example 54. Synthesis of Compound 54

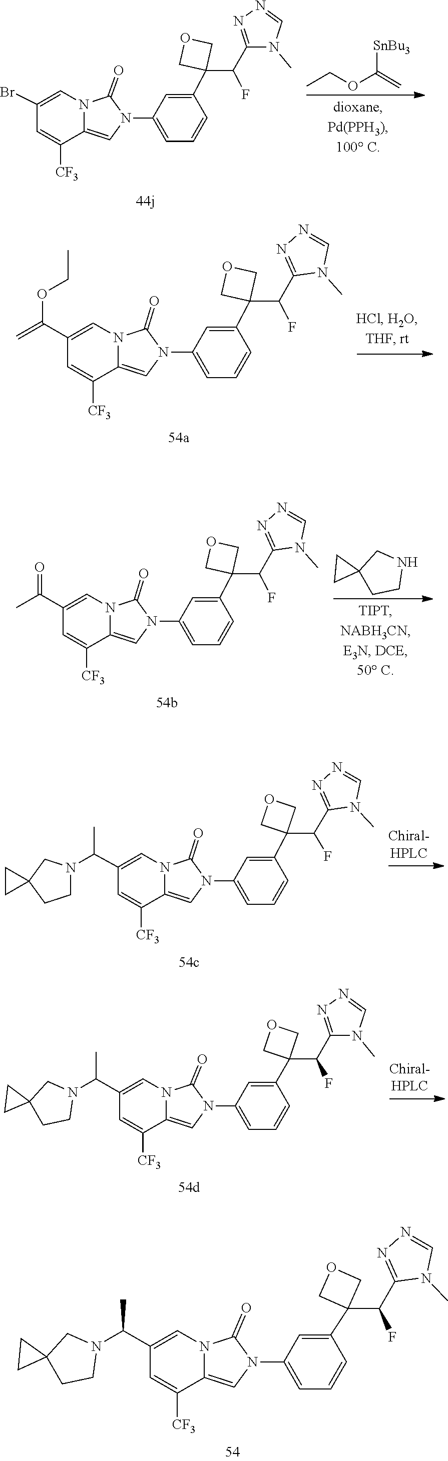

### Synthesis of 54a

To a stirred mixture of 44j (700.00 mg, 1,330 mmol, 1.00 equiv) and tributyl(1-ethoxyethenyl)stannane (720.54 ng, 1.995 mm ol, 1.5 equiv) in dioxane (7.00 mL) was added Pd(PPh₃)4 (153.70 mg, 0.133 mmol, 0.1 equiv) at room temperature. The resulting mixture was stirred overnight at 100° C. under nitrogen atmosphere. The mixture was allowed to cool down to room temperature. The resulting mixture was diluted with water (20 mL). The aqueous layer was extracted with CH₂CO₂ (3×20 mL). The resulting mixture was concentrated under reduced pressure. The residue was purified by prep-TLC (CH₂Cl₂/MeOH 12:1) to afford 54a (510 mg, 67.43%) as a yellow solid.

### Synthesis of 54b

To a stirred mixture of 54a (500.00 mag, 0.966 mmol, 1.00 equiv) in THE (5.00 mL) was added HCl (5.00 mL, 1M) dropwise at room temperature. The resulting mixture was stirred overnight at room temperature. The resulting mixture was diluted with water (20 mL). The aqueous layer was extracted with CH₂Cl₂ (3×30 mL). The resulting mixture was concentrated under reduced pressure. The residue was purified by prep-TLC (CH₂Cl₂/MeOH 10:1) to afford 54b (440 mg, 84.67%) as a yellow solid.

### Synthesis of 54c

To a stirred mixture of 54b (400.00 mg, 0.817 mmol, 1.00 equiv) and 5-azaspiro[2.4]heptane (119.11 mg, 1.226 mmol, 1.5 equiv) in DCE (4.00 mL) were added NaBH₃CN (154.08 mg, 2.452 mmol, 3 equiv), TEA (248.10 mg, 2.452 mmol, 3 equiv) and titanium(IV) isopropoxide (232.28 mg, 0.817 mmol, 1 equiv) at room temperature. The resulting mixture was stirred overnight at 50° C. The resulting mixture was diluted with water (15 mL) at room temperature. The aqueous layer was extracted with DCM (2×20 mL) The resulting mixture was concentrated under reduced pressure. The residue was purified by prep-TLC (CH₂Cl₂/MeOH 10:1) to afford 54c (180 mg, 37.83%) as a yellow solid.

### Synthesis of 54d

The 54c (180 mg) was separated by prep-Chiral HPLC with the following conditions (Column: CHIRAL ART Cellulose-SB, 2*25 cm, 5 um; Mobile Phase A: Hex (0.5% 2M NH₃-MeOH), Mobile Phase B: EtOH:DCM=1:1; Flow rate: 20 mL/min; Gradient: 30% B to 30% B in 9 min; Wave Length: 220/254 nm; RT1 (min): 5.66) to afford 54d (65 mg) as a yellow solid.

### Synthesis of 54

The 54d (65 mg) was separated by prep-Chiral HPLC with the following conditions (Column: CHIRALPAK IC, 2*25 cm, 5 μm; Mobile Phase A: Hex:DCM=3:1 (0.5% 2M NH₃-MeOH), Mobile Phase B: MeOH; Flow rate: 20 mL/min; Gradient: 40% B to 40% B in 16 min; Wave Length: 220/254 nm; RT1 (min): 10.09) to afford Compound 54 (22.8 mg) as a yellow solid.

LCMS: (ES, m/z): [M+H]⁺ 571

¹H NMR (400 MHz, DMSO, δ ppm): δ 0.40-0.58 (d, 4H), 1.02-1.32 (d, 3H), 1.74 (s, 2H), 2.35-2.41 (d, 1H), 2.52-2.63 (d, 2H), 2.67-2.80 (d, 1H), 3.14-3.33 (m, 4H), 4.80-4.94 (d, 1H), 5.16-5.25 (t, 2H), 5.25-5.40 (d, 1H), 6.16-6.40 (d, 1H), 7.09 (s, 1H), 7.13-7.15 (d, 1H), 7.34 (s, 1H), 7.42-7.44 (m, 1H), 7.55 (s, 1H), 7.68 (s, 1H), 7.79-7.83 (d, 1H), 8.38 (s, 1H).

### Example 55. Synthesis of Compound 55

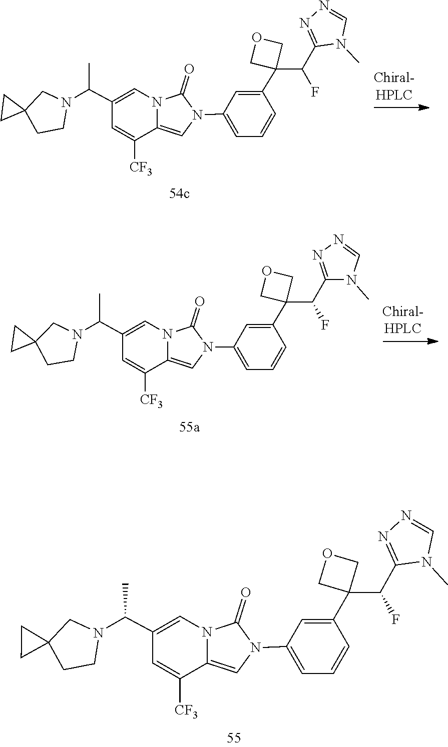 [SMILES: CC(c1cc(C(F)(F)F)c2cn(-c3cccc(C4(C(F)c5nncn5C)COC4)c3)c(=O)n2c1)N1CCC2(CC2)C1 ; CC(c1cc(C(F)(F)F)c2cn(-c3cccc(C4([C@@H](F)c5nncn5C)COC4)c3)c(=O)n2c1)N1CCC2(CC2)C1 ; C[C@H](c1cc(C(F)(F)F)c2cn(-c3cccc(C4([C@@H](F)c5nncn5C)COC4)c3)c(=O)n2c1)N1CCC2(CC2)C1]

### Synthesis of 55a

Compound 54c (180 mg) was separated by prep-Chiral HPLC with the following conditions (Column: CHIRAL ART Cellulose-SB, 2*25 cm, 5 μm; Mobile Phase A: Hex (0.5% 2M NH₃-MeOH), Mobile Phase B: EtOH:DCM=1:1; Flow rate: 20 mL/min; Gradient: 30% B to 30% B in 9 min; Wave Length: 220 nm; RT2 (min): 7.34) to afford 55a (65 mug) as a yellow solid.

### Synthesis of 55

The crude product (55a, 65 mg) was purified by prep-Chiral HPLC with the following conditions (Column: CHIRALPAK IC, 2*25 cm, 5 μm; Mobile Phase A: Hex:DCM=3:1 (0.5% 2M NH₃-MeOH), Mobile Phase B: MeOH; Flow rate: 20 mL/min; Gradient: 40% B to 40% B in 20 min; Wave Length: 220 nm; RT1 (min): 12.01) to afford Compound 55 (19.3 mg) as a yellow solid.

LCMS: (ES, m/z): [M+H]⁺ 571

¹H NMR: (400 MHz, DMSO, δ ppm): δ 0.45-0.58 (d, 4H), 1.20-1.30 (d, 3H), 1.74 (s, 2H), 2.35-2.41 (d, 1H), 2.52-2.63 (d, 2H), 2.68-2.80 (d, 1H), 3.12-3.25 (d, 1H), 3.28 (s, 3H), 4.76-4.94 (d, 1H), 5.13-5.28 (t, 2H), 5.28-5.40 (d, 1H), 6.22-6.33 (d, 1H), 7.09 (s, 1H), 7.13-7.15 (d, 1H), 7.34 (s, 1H), 7.41-7.45 (m, 1H), 7.55 (s, 1H), 7.68 (s, 1H), 7.77-7.79 (d, 1H), 8.38 (s, 1H).

### Example 56. Synthesis of Compound 56

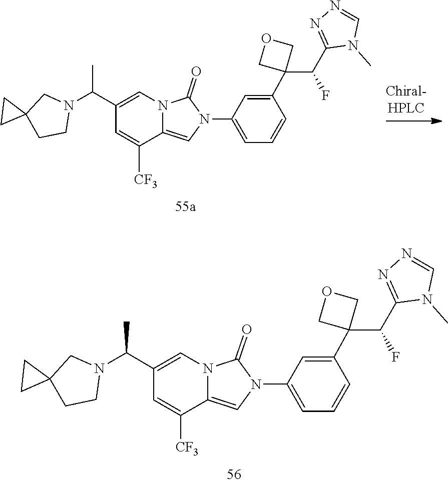 [SMILES: CC(c1cc(C(F)(F)F)c2cn(-c3cccc(C4([C@@H](F)c5nncn5C)COC4)c3)c(=O)n2c1)N1CCC2(CC2)C1 ; C[C@@H](c1cc(C(F)(F)F)c2cn(-c3cccc(C4([C@@H](F)c5nncn5C)COC4)c3)c(=O)n2c1)N1CCC2(CC2)C1]

### Synthesis of 56

Compound 55a was separated by prep-Chiral HPLC with the following conditions (Column: CHIRALPAK IC, 2*25 cm, 5 μm; Mobile Phase A: Hex:DCM=3:1 (0.5% 2M NH3-MeOH), Mobile Phase B: MeOH; Flow rate: 20 mL/min; Gradient: 40% B to 40% B in 20 min; Wave Length: 220/254 nm; RT2 (min): 15.44; the first peak is the product) to afford Compound 56 (20.6 mg) as a yellow solid,

LCMS: (ES, m/z): [M+H]⁺ 571.3

¹H NMR (400 MHz, DMSO, δ ppm): δ 0.40-0.58 (d, 4H), 1.20-1.31 (d, 3H), 1.74 (s, 2H), 2.31-2.40 (d, 1H), 2.52-2.60 (d, 2H), 2.68-2.81 (d, 1H), 3.15-3.25 (d, 1H), 3.25-3.30 (d, 3H), 4.86 (s, 1H), 5.10-5.29 (t, 2H), 5.29-5.42 (d, 1H), 6.22-6.34 (d, 1H), 7.08 (s, 1H), 7.13-7.15 (d, 1H), 7.33 (s, 1H), 7.41-7.46 (t, 1H), 7.55 (s, 1H), 7.68 (s, 1H), 7.77-7.79 (d, 1H), 8.37 (s, 1H).

### Example 57. Synthesis of Compound 57

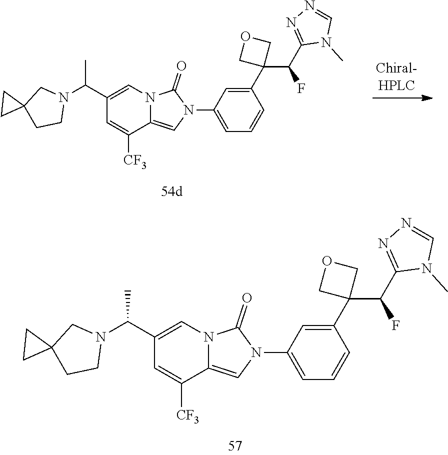 [SMILES: CC(c1cc(C(F)(F)F)c2cn(-c3cccc(C4([C@H](F)c5nncn5C)COC4)c3)c(=O)n2c1)N1CCC2(CC2)C1 ; C[C@H](c1cc(C(F)(F)F)c2cn(-c3cccc(C4([C@H](F)c5nncn5C)COC4)c3)c(=O)n2c1)N1CCC2(CC2)C1]

### Synthesis of 57

Compound 54d (65 mg) was purified by prep-Chiral HPLC with the following conditions (Column: CHIRALPAK IC, 2*25 ca, 5 μm; Mobile Phase A: Hex:DCM=3:1 (0.5% 2M NH3-MeOH), Mobile Phase B: MeOH; Flow rate: 20 mL/min; Gradient: 40% B to 40% B in 16 min; Wave Length: 220/254 nm; RT2 (min): 12.95) to afford Compound 57 (20.0 mg) as a yellow solid.

LCMS: (ES, m/z): [M+H]⁺ 571.3

¹H NMR (400 MHz, DMSO, δ ppm): δ 0.40-0.58 (d, 4H), 1.02-1.32 (d, 3H), 1.74 (s, 2H), 2.35-2.41 (d, 1H), 2.52-2.63 (d, 2H), 2.67-2.80 (d, 1H), 3.14-3.30 (d, 4H), 4.86 (s, 1H), 5.16-5.25 (t, 2H), 5.25-5.40 (d, 1H), 6.16-6.40 (d, 1H), 7.00-7.11 (d, 1H), 7.11-7.19 (d, 1H), 7.33 (s, 1H), 7.39-7.48 (t, 1H), 7.56 (s, 1H), 7.68 (s, 1H), 7.72-7.83 (d, 1H), 8.38 (s, 1H).

### Example 58. Synthesis of Compound 58

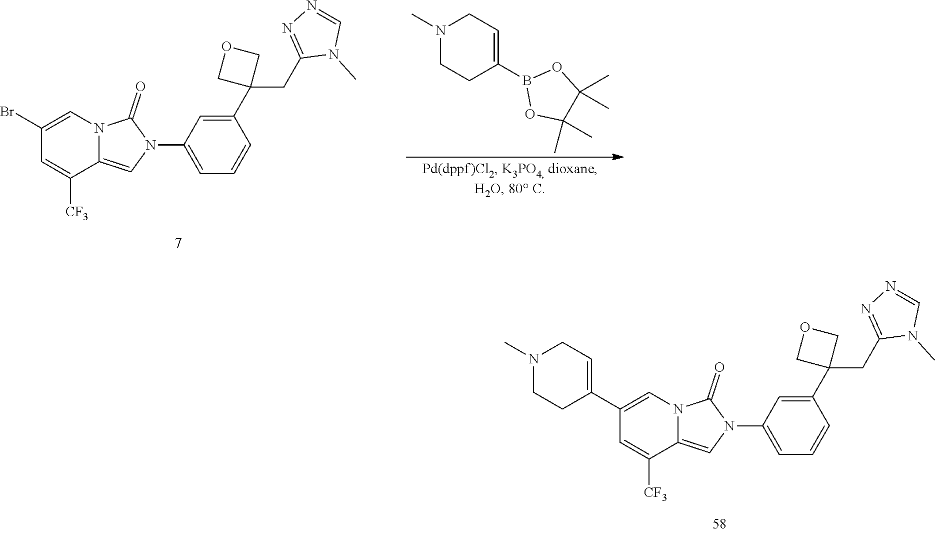

### Synthesis of 58

To a stirred mixture of Compound 7 (100.00 mg, 0.197 mmol, 1.00 equiv) and 1-methyl-4-(4,4,5,5-tetramethyl-1,3,2-dioxaborolan-2-yl)-3,6-dihydro-2H-pyridine (65.84 mg, 0.296 mmol, 1.50 equiv), K₃PO₄ (83.52 mg, 0.394 mmol, 2.00 equiv) in dioxane (2.00 mL) and H₂O (0.50 ml) was added Pd(dppf)Cl₂ (14.40 mg, 0.020 mmol, 0.10 equiv) under argon atmosphere. The resulting mixture was stirred for 2 h at 80° C. under argon atmosphere. The resulting mixture was diluted with water (10 mL). The aqueous layer was extracted with EtOAc (2×10 mL). The resulting mixture was concentrated under reduced pressure. The residue was purified by prep-TLC, eluted with CH₂Cl₂/MeOH (50:1). The crude was dissolved in CH₂Cl₂ (10 mL). To the above mixture was added SiliaMetS thiol (200 mg). The resulting mixture was stirred overnight. The resulting mixture was filtered, the filter cake was washed with CH₂Cl₂ (5 mL). The filtrate was concentrated under reduced pressure. The crude product was re-crystallized from MeOH to afford 2-(3-[3-[(4-methyl-1,2,4-triazol-3-yl)methyl]oxetan-3-yl]phenyl)-6-(1-methyl-3,6-dihydro-2H-pyridin-4-yl)-8-(trifluoromethyl)imidazo[1,5-a]pyridin-3-one (Compound 58, 57 mg, 55.23%) as a yellow solid,

LCMS: (ES, m/z): [M+H]⁺ 525

¹H NMR: (400 MHz, DMSO-d6, ppm): δ 2.24-3.01 (s, 3H), 2.39-2.44 (m, 2H), 2.54-2.56 (m, 2H), 2.92 (s, 3H), 2.97-3.02 (m, 2H), 3.54 (s, 2H), 4.89-4.96 (m, 4H), 6.32 (s, 1H), 6.88-6.90 (d, 1H), 7.36-7.43 (m, 4H), 7.55 (s, 1H), 7.75-7.77 (d, 1H), 8.21 (s, 1H).

### Example 59. Synthesis of Compound 59

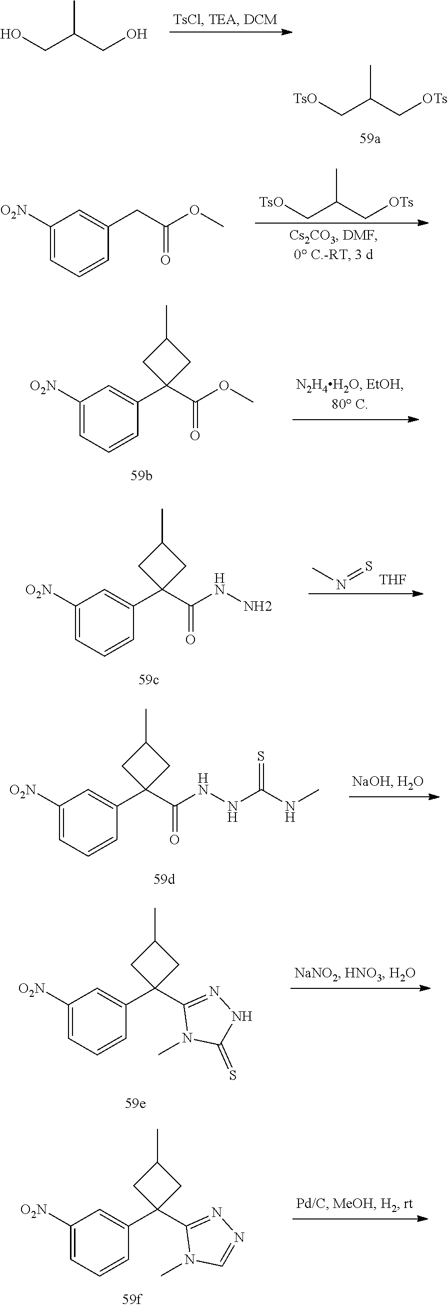

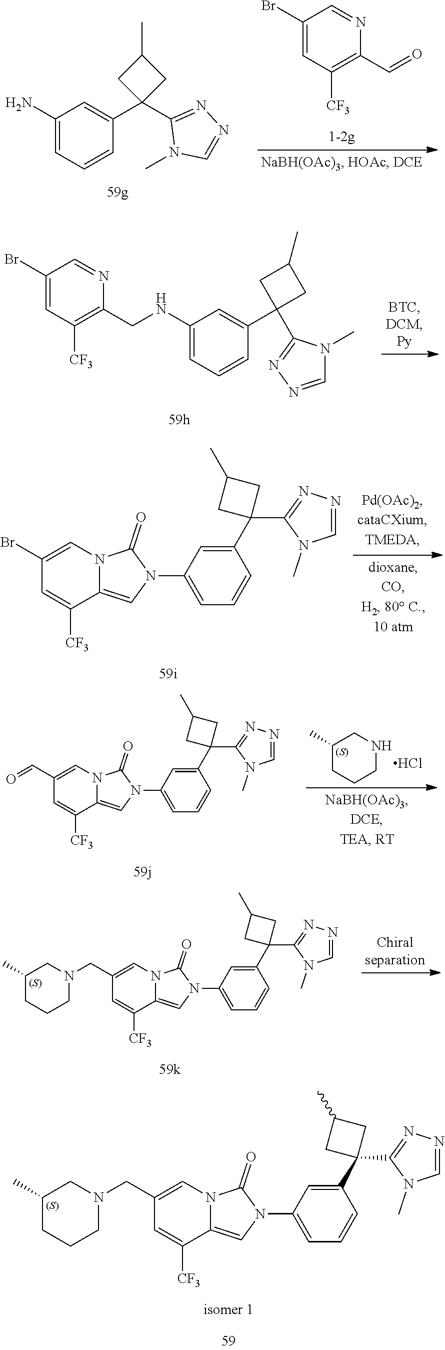

### Synthesis of 59a

To a stirred solution of methylpropanediol (5 g, 55,480 mmol, 1 equiv) and Et₃N (5.61 g, 55.480 mmol, 1 equiv) in DCM (50 ml) were added TsCl (21.15 g, 110.960 mmol, 2 equiv) dropwise at 0° C. The resulting mixture was stirred overnight at room temperature. The reaction was quenched by the addition of water (500 mL) at room temperature. The aqueous layer was extracted with CH₂Cl₂ (2×150 mL). The resulting mixture was concentrated under reduced pressure. The residue was purified by silica gel column chromatography, elated with PE/EA=50:1 to afford 59a (15 g, 63.10%) as a brown solid.

### Synthesis of 59b

To a stirred mixture of methyl 2-(3-nitrophenyl)acetate (5 g, 25,618 mmol, 1 equiv) in DMF (50 mL) was added Cs₂CO₃ (41.74 g, 128.090 mmol, 5.0 equiv) at 0° C. under nitrogen atmosphere. The resulting mixture was stirred for 1 b at 0° C. under nitrogen atmosphere. To the above mixture was added 59a (15.31 g, 38.427 mmol, 1.5 equiv) at 0° C. The resulting mixture was stirred for additional 2 days at room temperature. The reaction was quenched by the addition of 200 mL NH₄Cl (aq.) at 0° C. The aqueous layer was extracted with EtOAc (2×100 mL). The resulting mixture was concentrated under reduced pressure. The residue was purified by silica gel column chromatography, eluted with PE/EA=100:1 to afford 59b (1 g, 14.09%) as a white solid.

### Synthesis of 59c

To a stirred solution of 59b (1 g, 4,012 mmol, 1 equiv) in EtOH (10 mL) were added NH₂NH₂H₂O (2.01 g, 40,120 mmol, 10 equiv) at room temperature. The resulting mixture was stirred overnight at 80° C. The reaction was quenched by the addition of H₂O (100 mL) at room temperature. The aqueous layer was extracted with CH₂Cl₂:MeOH=10:1 (3×30 mL). The resulting mixture was concentrated under reduced pressure. The residue was purified by Prep-TLC (CH₂Cl₂/MeOH=15:1) to afford 59c (900 mg, 87.30%) as a white solid.

### Synthesis of 59d

To a stirred mixture of 59c (880 mg, 3.530 mmol, 1 equiv) in tetrahydrofuran (9 ml) was added methyl isothiocyanate (516.20 mg, 7.060 mmol, 2 equiv) at room temperature. The resulting mixture was stirred overnight at room temperature. The reaction was quenched by the addition of water (30 mL) at room temperature. The resulting mixture was concentrated under reduced pressure. The precipitated solids were collected by filtration and washed with water (10 mL). The resulting solid was dried under vacuum to afford 59d (900 mg, 73.54%) as a yellow solid.

### Synthesis of 59e

To a stirred solution of 59d (900 mg, 2,792 mmol, 1 equiv) was added NaOH (558.31 mg, 13,960 mmol, 5 equiv) in 7 mL of H₂O at room temperature. The resulting mixture was stirred overnight at room temperature. The mixture was neutralized to pH 5 with HCl (aq.) (1 M). The aqueous layer was extracted with CH₂Cl₂:MeOH=10:1 (3×5 mL). The residue was purified by Prep-TLC (CH₂Cl₂/MeOH=15:1) to afford 59e (700 mg, 79.08%) as a white solid,

### Synthesis of 59f

To a stirred solution of 59e (700 mg, 2,300 mmol, 1 equiv) and NaNO₂ (1586.78 mg, 23,000 mmol, 10 equiv) in 1120 (7 mL) were added HNO₃ (23 mL, 1 M, 23,000 mmol, 10 equiv) at 0° C. The resulting mixture was stirred overnight at room temperature. The reaction was quenched by the addition of NaHCO₃ (20 m-L) at 0° C. The aqueous layer was extracted with CH₂Cl₂:MeOH=10:1 (2×10 mL). The combined organic layers were concentrated under reduced pressure. The residue was purified by silica gel column chromatography, eluted with CH₂Cl₂/MeOH=15:1 to afford 59f (600 mg, 91.97%) as a white solid.

### Synthesis of 59g

To a solution of 59f (600 mg, 2.203 mmol, 1 equiv) in 20 ml, MeOH was added Pd/C (10%, 60 mg) under nitrogen atmosphere in a 100 mL round-bottom flask. The mixture was hydrogenated at room temperature overnight under hydrogen atmosphere using a hydrogen balloon, filtered through a Celite pad and concentrated under reduced pressure to afford 59g (500 mg, 88.03%) as a yellow solid.

### Synthesis of 59h

To a stirred solution of 59g (480 mg, 1,981 mmol, 1.00 equiv) and I-2g (754.70 mg, 2.972 mmol, 1.5 equiv) in DCE (5 mL) was added HOAc (118.95 mg, 1.981 mmol, 1 equiv) and NaBH(OAc)3 (1259.44 mg, 5.943 mmol, 3 equiv) at room temperature. The resulting mixture was stirred overnight at room temperature. The reaction was quenched by the addition of 10 mL of H₂O at room temperature. The aqueous layer was extracted with CH₂Cl₂ (2×10 mL). The resulting mixture was concentrated under vacuum. The residue was purified by Prep-TLC (CH₂Cl₂/MeOH=20:1) to afford 59h (500 mg, 50.45%) as a yellow solid.

### Synthesis of 59i

To a stirred solution of 59h (480 mg, 1.763 mmol, 1 equiv) and pyridine (836.58 mg, 10.578 mmol, 6 equiv) in DCM (5 mL) was added triphosgene (261.54 mg, 0.881 mmol, 0.5 equiv) in one portion at 0° C. The reaction was quenched by the addition of 30 mL NaHCO₃ (aq.) at room temperature. The aqueous layer was extracted with CH₂Cl₂ (2×10 mL). The resulting mixture was concentrated under reduced pressure. The residue was purified by Prep-TLC (CH₂Cl₂/MeOH=15:1) to afford 59i (400 mg, 84.28%) as a yellow solid.

### Synthesis of 59j

To a stirred solution of 59i (14 g, 27.650 mmol, 1 equiv) and TMEDA (6.43 g, 55.300 mmol, 2 equiv) in dioxane (500 mL) were added Butyldi-1-adamantylphosphine (1.985 g, 5.530 mmol, 0.2 equiv) and Pd(OAc)2 (0.62 g, 2.765 mmol, 0.1 equiv) at room temperature under nitrogen atmosphere. The mixture was purged with nitrogen and then was pressurized to 10 atm with CO and H₂ (1:1) at 80° C. overnight. The resulting mixture was concentrated under vacuum. The residue was purified by silica gel column chromatography, eluted with CH₂Cl₂/MeOH=20:1 to afford 59j (8 g, 60.35%) as an orange solid.

### Synthesis of 59k

To a stirred mixture of 59j (800 mg, 1.757 mmol, 1.00 equiv) and (3S)-3-methylpiperidine hydrochloride (476.51 mg, 3,514 mmol, 2 equiv) in DCE (8 mL) was added Et₃N (533.25 mg, 5.271 mmol, 3 equiv) at room temperature. The resulting mixture was stirred for 30 min at room temperature. To the above mixture was added NaBH(OAc)₃ (1116.84 mg, 5,271 mmol, 3 equiv) at room temperature. The resulting mixture was stirred overnight at room temperature. The reaction was quenched by the addition of water (50 ML) at room temperature. The aqueous layer was extracted with CH₂Cl₂ (2×30 mL). The resulting mixture was concentrated under reduced pressure. The residue was purified by Prep-TLC (CH₂Cl₂/MeOH=15:1) to afford the crude product. The crude product (500 mg) was purified by Prep-HPLC with the following conditions (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 44% B to 73% B in 8 ruin, Wave Length: 220 nm; RT1 (min): 7.68) to afford 59k (400 mg, 41.43%) as a yellow solid.

### Synthesis of 59

59k (400 mg) was purified by chiral separation with the following conditions (Column: CHIRALPAK AD-H, 2*25 cm, 5 μm; Mobile Phase A: Hex (0.5% 2M NH₃-MeOH), Mobile Phase B: EtOH; Flow rate: 20 mL/min; Gradient: 15% B to 15% B in 20 min; Wave Length: 220/254 nm; RT1 (min): 9.69; RT2 (min): 13.84; the first peak is product) to afford Compound 59 (216.6 mg, 42.40%) as a yellow solid.

LCMS: (ES, m/z): [M+H]⁺ 539.

H-NMR: (400 MHz, DMSO-d₆, ppm, δ): 0.78-0.95 (d, 4H), 1.05-1.16 (d, 3H), 1.37-1.53 (m, 1H), 1.53-1.70 (m, 4H), 1.89 (s, 1H), 2.54-2.56 (s, 3H), 2.72-2.80 (m, 2H), 2.90 (s, 2H), 3.21 (s, 3H), 3.24 (s, 2H), 7.00 (s, 1H), 7.24-7.26 (d, 1H), 7.37 (s, 1H), 7.47-7.52 (m, 1H), 7.63-7.65 (m, 2H), 7.84 (s, 1H), 8.29 (s, 1H).

Alternatively, Compound 59 may be also prepared in the manner outlined below:

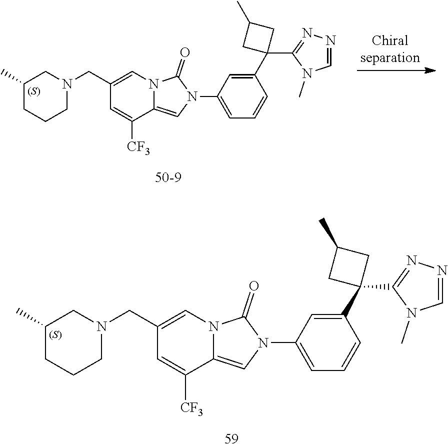

### 1. Synthesis of 59

50-9 (11.5 g) was purified by Prep-SFC with the following conditions (Column: CHIRALPAK AD-H, 5*25 cm, 5 μm; Mobile Phase A: C02, Mobile Phase B: ETOH (0.1% 2M NH₃-MEOH); Flow rate: 200 mL/min; Gradient: isocratic 40% B; Column Temperature (° C.): 35; Back Pressure (bar): 100; Wave Length: 220 nm; RT1 (min): 3.55; RT2 (min): 4.71; the first peak is product) to afford 59 (5.7744 g, 53.79%) as a yellow solid. LC-MS-: (ES, m/z): [M+H]⁺ 539 H-NMR: (400 MHz, DMSO-d6, δ ppm): 0.79-0.92 (m, 4H), 0.98-1.15 (d, 3H), 1.35-1.52 (m, 1H), 1.55-1.71 (m, 4H), 1.81-1.93 (m, 1H), 2.50-2.61 (m, 3H), 2.69-2.77 (m, 2H), 2.81-2.93 (m, 2H), 3.15-3.22 (m, 4H), 6.99 (s, 1H), 7.23-7.25 (d, 1H), 7.35 (s, 1H), 7.47-7.51 (m, 1H), 7.64-7.66 (m, 2H), 7.85 (s, 1H), 8.29 (s, 1H).

### Example 60. Synthesis of Compound 60

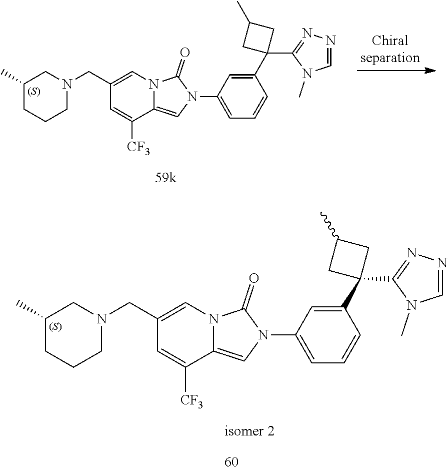

### Synthesis of 60

59k (400 mg) was purified by chiral separation with the following conditions (Column: CHIRALPAK AD-H, 2*25 cm, 5 μm; Mobile Phase A: Hex (0.5% 2M NH₃-MeOH), Mobile Phase B: EtOH; Flow rate: 20 mL/min; Gradient: 15% B to 15% B in 20 min; Wave Length: 220/254 nm; RT1 (min): 9.69; RT2 (min): 13.84; the second peak is product) to afford Compound 60 (97.3 mg, 19.05%) as a yellow solid.

LCMS: (ES, m/z): [M+H]⁺ 539.

H-NMR: (400 MHz, DMSO-d₆, ppm, δ): 0.75-0.98 (d, 4H), 1.05-1.28 (d, 3H), 1.35-1.45 (m, 1H), 1.45-1.66 (m, 4H), 1.89 (s, 1H), 2.25-2.34 (m, 2H), 2.34-2.41 (m, 1H), 2.75 (s, 2H), 3.10-3.14 (m, 2H), 3.25 (s, 5H), 6.98-7.02 (m, 1H), 7.08-7.10 (m, 1H), 7.36 (s, 1H), 7.45-7.49 (m, 1H), 7.60-7.67 (m, 2H), 7.70 (s, 1H), 8.36 (s, 1H).

### Example 61. Synthesis of Compound 61

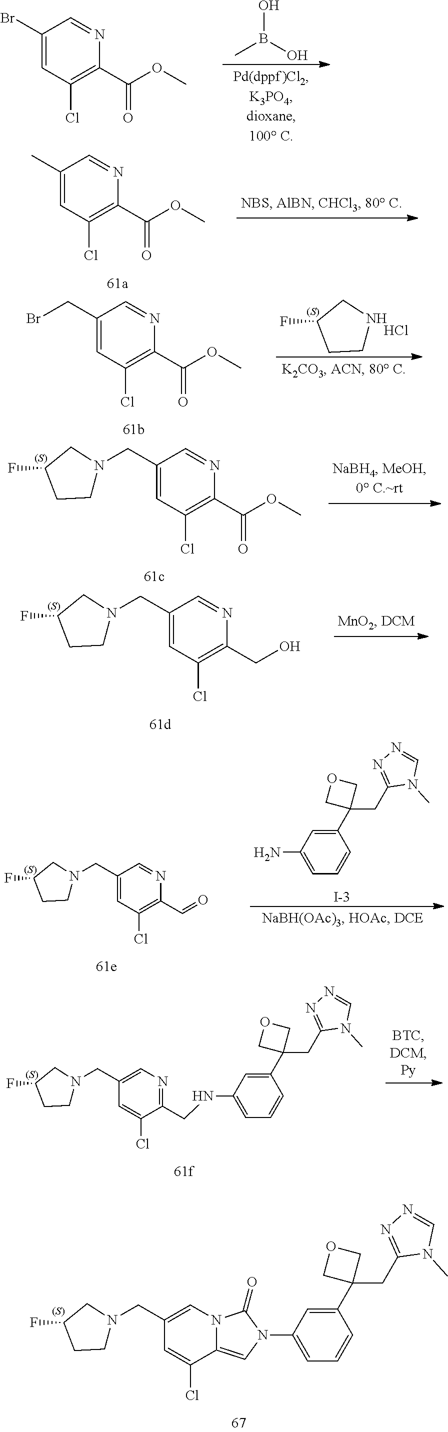

### Synthesis of 61a

To a stirred solution of methyl 5-bromo-3-chloropyridine-2-carboxylate (30.00 g 119.770 mmol, 1.00 equiv) and methylboronic acid (21.51 g, 359.310 mmol, 3 equiv) in dioxane (350.00 mL) were added K₃PO₄ (50.85 g, 239,540 mmol, 2 equiv) and Pd(dppf)Cl₂ (8.76 g, 11,977 mmol, 0.1 equiv) at room temperature under nitrogen atmosphere. The resulting mixture was stirred overnight at 80 degrees C. under nitrogen atmosphere. The reaction was quenched with water at room temperature. The aqueous layer was extracted with EtOAc (3×600 mL). The resulting mixture was concentrated under reduced pressure. The residue was purified by silica gel column chromatography, eluted with PE/EtOAc (20:1) to afford 61a (14 g, 56.68%) as an off-white oil.

### Synthesis of 61b

To a stirred solution of 61a (3.00 g, 16.163 mmol, 1.00 equiv) and NBS (4.32 g, 24.272 mmol, 1.50 equiv) in CHCl₃ (30.00 mL) were added AIBN (7.96 g, 48.489 mmol, 3 equiv) at room temperature under nitrogen atmosphere. The resulting mixture was stirred overnight at 80 degrees C. under nitrogen atmosphere. The reaction was quenched with water at room temperature. The aqueous layer was extracted with EtOAc (3×80 mL). The resulting mixture was concentrated under reduced pressure. The residue was purified by silica gel column chromatography, eluted with PE/EtOAc (30:1) to afford 61b (1.5 g, 21.05%) as an off-white oil,

### Synthesis of 61c

To a stirred solution of 61b (1.50 g, 5.671 mmol, 1.00 equiv) and (3S)-3-fluoropyrrolidine hydrochloride (1.42 g, 11.342 mmol, 2 equiv) in ACN (15.00 mL) was added K₂CO₃ (1.57 g, 11.342 mmol, 2 equiv) at room temperature. The resulting mixture was stirred overnight at 80 degrees C. under nitrogen atmosphere. The reaction was quenched with water at room temperature. The aqueous layer was extracted with EtOAc (3×50 mL). The resulting mixture was concentrated under reduced pressure. The residue was purified by silica gel column chromatography, eluted with PE/EtOAc (5:1) to afford 61c (800 mg, 50.18%) as an off-white solid.

### Synthesis of 61d

To a stirred solution of 61c (800.00 mg, 2.934 mmol, 1.00 equiv) in MeOH (10.00 mL) was added NaBH₄ (221.98 mg, 5.867 mmol, 2 equiv) at 0 degrees C. The resulting mixture was stirred for 5 h at room temperature. The reaction was quenched with NH₄Cl (aq.) at room temperature. The aqueous layer was extracted with EtOAc (3×10 mL). The resulting mixture was concentrated under reduced pressure. The residue was purified by Prep-TLC (PE/EA 1:1) to afford 61d (500 mg, 65.48%) as a white oil.

### Synthesis of 61e

To a stirred solution of 61d (500.00 mg, 2.043 mmol, 1.00 equiv) in DCM (7.00 mL) was added MnO₂ (1776.47 mg, 20.434 mmol, 10 equiv) at room temperature. The resulting mixture was stirred overnight at 40 degrees C. The resulting mixture was filtered and the filter cake was washed with DCM (3×10 mL). The filtrate was concentrated under reduced pressure. The residue was purified by Prep-TLC (PE/EA 2:1) to afford 61e (400 mg, 75.82%) as a white oil.

### Synthesis of 61f

To a stirred solution of 61e (400.00 mg, 1,648 mmol, 1.00 equiv) in DC. (6.00 mL) was added I-3 (402.67 mg, 1.648 mmol, 1 equiv) at room temperature. The resulting mixture was stirred for 1h at room temperature. To the above mixture was added NaBH(OAc)₃ (1048.00 mg, 4,945 mmol, 3 equiv) and HOAc (9.9 mg, 0.165 mmol, 0.1 equiv) at room temperature. The resulting mixture was stirred for additional 2h at room temperature. The reaction was quenched with NH₄Cl (aq.) at room temperature. The aqueous layer was extracted with DCM (3×20 mL). The resulting mixture was concentrated under reduced pressure. The residue was purified by Prep-TLC (CH₂Cl₂/MeOH 15:1) to afford 61f (500 mg, 62.48%) as a white solid,

### Synthesis of 61

To a stirred solution of 61f (35.00 mg, 0.074 mmol, 1.00 equiv) and Pyridine (35.27 mg, 0.446 mmol, 6 equiv) in DCM (1.00 mL) was added Triphosgene (7:72 mg, 0.026 mmol, 0.35 equiv) at 0 degrees C. The resulting mixture was stirred for 30 min at 0 degrees C. The reaction was quenched with NaHCO₃ (aq.) (10.00 mL) at room temperature. The aqueous layer was extracted with DCM (3×8 mL). The resulting mixture was concentrated under vacuum. The residue was purified by reverse flash chromatography with the following conditions: column, C18 silica gel; mobile phase, A: water (0.5% NH₄HCO₃), B: CH₃CN, 30% B to 50% B gradient in 30 min; detector, UV 254 nm. This resulted in 61 (2.3 ng, 6.10%) as a yellow solid.

LC-MS: (ES, m/z): [M+H]⁺ 497

H-NMR: (400 MHz, CDCl₃, ppm, δ): 2.40-2.48 (m, 2H), 2.95 (s, 3H), 3.26-3.39 (m, 2H), 3.62 (s, 2H), 3.72-3.78 (m, 2H), 4.06 (s, 2H), 5.07-5.09 (d, 2H), 5.13-5.14 (d, 2H), 5.36-5.49 (d, 1H), 6.79 (s, 1H), 6.88-6.91 (d, 2H), 7.16 (s, 1H), 7.42-7.48 (m, 2H), 7.65 (s, 1H), 8.12 (s, 1H).

### Example 62. Synthesis of Compound 62

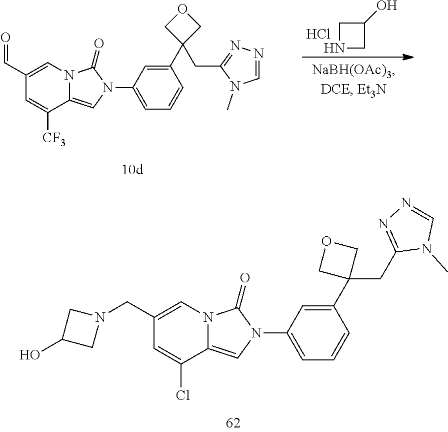 [SMILES: Cn1cnnc1CC1(c2cccc(-n3cc4c(C(F)(F)F)cc(C=O)cn4c3=O)c2)COC1 ; Cl ; OC1CNC1 ; Cn1cnnc1CC1(c2cccc(-n3cc4c(Cl)cc(CN5CC(O)C5)cn4c3=O)c2)COC1]

### Synthesis of 62

To a stirred mixture of 10d (200 mg, 0.437 mmol, 1 equiv) and azetidin-3-ol hydrochloride (143.70 mg, 1.311 mmol, 3 equiv) in DCE (2 mL) were added Et₃N (44.24 mg, 0.437 mmol, 1 equiv) and NaBH(OAc)₃ (185.34 mg, 0,874 mmol, 2 equiv) at room temperature. The resulting mixture was stirred overnight at room temperature. The reaction was quenched by the addition of sat NH₄Cl (aq.) (10 mL) at room temperature. The aqueous layer was extracted with CH₂Cl₂ (3×10 mL). The resulting mixture was concentrated under reduced pressure. The residue was purified by Prep-TLC (CH₂Cl₂/MeOH 9:1) to afford 62 (150 mg, crude). The crude product (150 mg) was purified by Prep-HPLC with the following conditions (Column: XBridge Prep OBD C18 Column, 30*150 mm, 5m; Mobile Phase A: Water (10 mmol/L NH₄CO₃+0.1% NH₃·H₂O), Mobile Phase B: ACN; Flow rate: 60 mL/min; Gradient: 10% B to 40% B in 8 min; Wave Length: 220 nm; RT1 (min): 7.23) to afford Compound 62 (23.1 mg, 10.27%) as a yellow solid.

LC-MS: (ES, m/z): [M+H]⁺ 515

H-NMR: (400 MHz, DMSO, δ ppm): 2.67-2.96 (m, 2H), 2.96-3.00 (d, 3H), 3.33-3.35 (d, 2H), 3.46-3.53 (m, 4H), 4.18-4.23 (m, 1H), 4.88-4.96 (m, 4H), 5.32-5.34 (d, 1H), 6.87-6.89 (d, 1H), 6.96 (s, 1H), 7.31 (s, 1H), 7.38-7.41 (m, 2H), 7.64 (s, 1H), 7.74-7.76 (m, 1H), 8.20 (s, 1H).

### Example 63. Synthesis of Compound 63

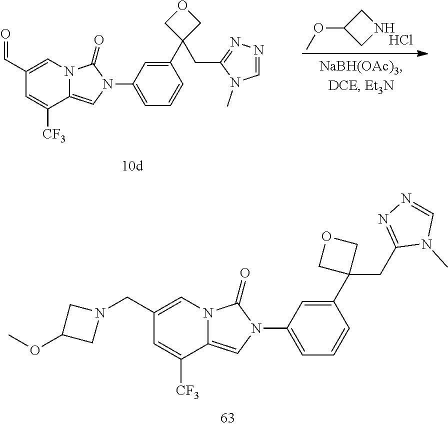

### Synthesis of 63

To a stirred mixture of 10d (200 mg, 0.437 mmol, 1 equiv) and 3-methoxyazetidine hydrochloride (162.10 mg, 1.311 mmol, 3 equiv) in DCE (2 mL) were added Et₃N (176.96 mg, 1:748 mmol, 4 equiv) and NaBH(OAc)3 (185.34 mg, 0,874 mmol, 2 equiv) at room temperature under nitrogen atmosphere. The resulting mixture was stirred overnight at room temperature under nitrogen atmosphere. The reaction was quenched with water (20 ml) at room temperature. The aqueous layer was extracted with CH₂Cl₂ (3×10 mL). The resulting mixture was concentrated under reduced pressure. The residue was purified by Prep-TLC (CH₂Cl₂/MeOH 10:1) to afford crude product (150 mg). The crude product (150 mg) was purified by Prep-HPLC with the following conditions (Column: XBridge Prep OBD C18 Column, 30*150 mm, 5|1m; Mobile Phase A: Water (10 mmol/L NH₄HCO₃+0.1% NH₃·H₂O), Mobile Phase B: ACN; Flow rate: 60 mL/min; Gradient: 10% B to 40% B in 8 min; Wave Length: 220 nm; RT1 (min): 7.23. This resulted in Compound 63 (63.6 mg, 27.52%) as a yellow solid.

LC-MS: (ES, m/z): [M+H]⁺ 529

H-NMR: (400 MHz, DMSO, ppm): 52.86-2.89 (m, 2H), 2.96 (s, 3H), 3.00 (s, 3H), 3.15 (m, 2H), 3.33-3.38 (d, 2H), 3.45-3.53 (m, 2H), 3.95-3.99 (m, 1H), 4.90-4.96 (m, 4H), 6.87-6.89 (d, 1H), 6.97 (s, 1H), 7.31 (s, 1H), 7.38-7.42 (m, 2H), 7.67 (s, 1H), 7.74-7.76 (m, 1H), 8.20 (s, 1H).

### Example 64. Synthesis of Compound 64

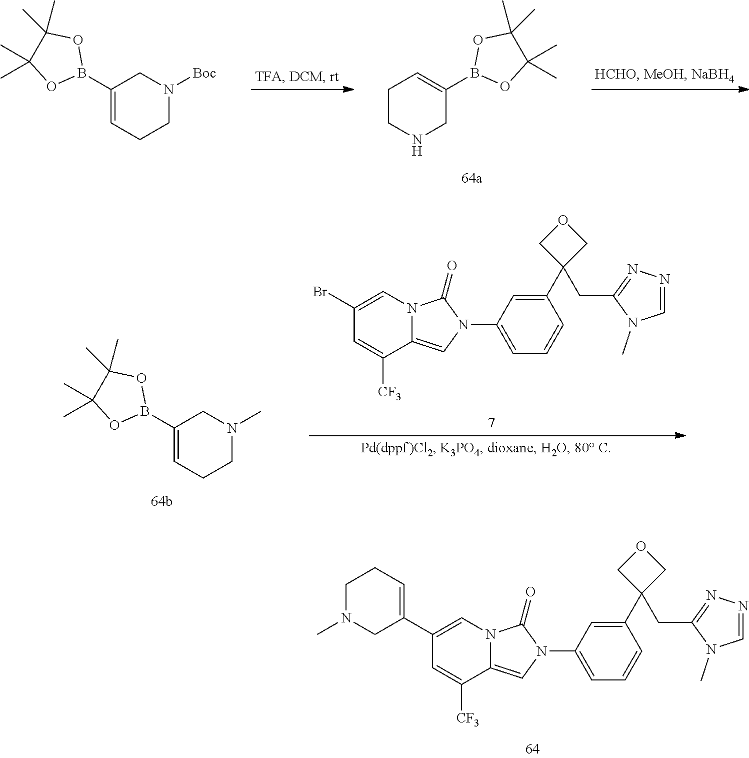

### Synthesis of 64a

Into a 100 mL round-bottom flask were added tert-butyl 3-(4,4,5,5-tetramethyl-1,3,2-dioxaborolan-2-yl)-5,6-dihydro-2H-pyridine-1-carboxylate (2 g) and TFA (5 mL) in DCM (15 mL) at room temperature. The resulting mixture was stirred for 2 h at room temperature. The resulting mixture was concentrated under reduced pressure. This resulted in 64a (1g, 73.94%) as colorless oil,

### Synthesis of 64b

A solution of 64a (1.00 g, 4.782 mmol, 1.00 equiv) and formaldehyde solution (1.16 g, 14.346 mmol, 3.00 equiv, 37%) in MeOH (10.00 mL) was stirred overnight at room temperature under nitrogen atmosphere. To the above mixture was added NaBH₄ (0.36 g, 9.516 mmol, 1.99 equiv). The resulting mixture was stirred for 5h at room temperature under nitrogen atmosphere. The reaction was quenched with NH₄Cl (aq.) (100 mL) at room temperature. The aqueous layer was extracted with EtOAc (3×30 mL). The resulting mixture was concentrated under reduced pressure. This resulted in 64b (500 mg, 46.86%) as colorless oil

### Synthesis of 64

To a stirred mixture of 64b (200.00 mg, 0.393 mmol, 1.00 equiv) and 7 (131.69 mg, 0.590 mmol, 1.50 equiv) in dioxane (1.60 mL) and 120 (0.4 mL) were added Pd(dppf)Cl₂ (57.58 mg, 0.079 mmol, 0.20 equiv) and K₃PO₄ (167.04 mg, 0.787 mmol, 2.00 equiv) at room temperature under nitrogen atmosphere. The resulting mixture was stirred overnight at 80 degrees C. under nitrogen atmosphere. The reaction was quenched with water (50 mL) at room temperature. The aqueous layer was extracted with EtOAc (3×30 mL). The resulting mixture was concentrated under reduced pressure. The product was dissolved in DCM (10 mL) and isolute Si-thiol (100 mg) was added. The mixture was stirred for 30 min. The resulting mixture was filtered. The filtrate was concentrated under reduced pressure. The crude product (100 ng) was purified by Prep-HPLC with the following conditions (Column: YMC-Actus Triart C18 ExRS, 30*150 mm, 5 μm; Mobile Phase A: water (10 mmol/L NH₄HCO₃), Mobile Phase B: ACN; Flow rate: 60 mL/min; Gradient: 20% B to 43% B in 8 min; Wave Length: 254/220 nm; RT1 (min): 8.22) to afford Compound 64 (36.7 mg, 17.78%) as a yellow solid.

LC-MS: (ES, m/z): [M+H]⁺ 525

H-NMR: (400 MHz, DMSO, δ ppm): 32.29 (s, 2H), 2.34 (s, 3H), 2.44-2.49 (m, 2H), 2.97 (s, 3H), 3.15 (s, 2H), 3.57 (s, 2H), 4.91-4.96 (m, 4H), 6.35 (s, 1H), 6.88-6.90 (d, 1H), 7.33-7.36 (d, 1H), 7.39-7.41 (d, 1H), 7.42-7.43 (m, 2H), 7.51 (s, 1H), 7.75-7.77 (d, 1H), 8.21 (s, 1H).

### Example 65. Synthesis of Compound 65

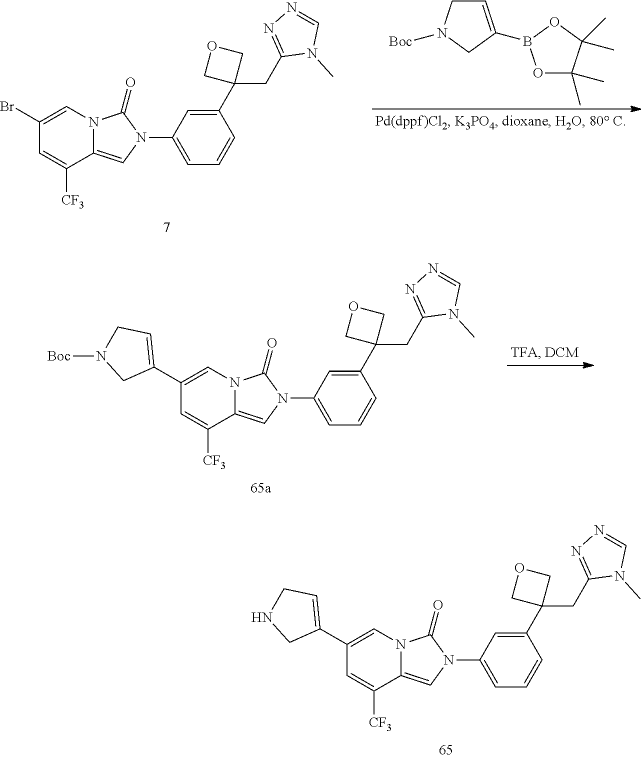 [SMILES: Cn1cnnc1CC1(c2cccc(-n3cc4c(C(F)(F)F)cc(Br)cn4c3=O)c2)COC1 ; CC(C)(C)OC(=O)N1CC=C(B2OC(C)(C)C(C)(C)O2)C1 ; Cn1cnnc1CC1(c2cccc(-n3cc4c(C(F)(F)F)cc(C5=CCN(C(=O)OC(C)(C)C)C5)cn4c3=O)c2)COC1 ; Cn1cnnc1CC1(c2cccc(-n3cc4c(C(F)(F)F)cc(C5=CCNC5)cn4c3=O)c2)COC1]

### Synthesis of 65a

To a stirred mixture of 7 (200.00 mg, 0.393 mmol, 1.00 equiv), tert-butyl 3-(4,4,5,5-tetramethyl-1,3,2-dioxaborolan-2-yl)-2,5-dihydropyrrole-1-carboxylate (174.22 mg, 0.590 mmol, L 50 equiv) and K₃PO₄ (167.04 mg, 0.786 mmol, 2.00 equiv) in dioxane (4.00 mL) and 120 (1.00 mL) was added Pd(dppf)Cl₂ (28.79 mg, 0.039 mmol, 0.10 equiv) under argon atmosphere. The resulting mixture was stirred overnight at 80 degrees C. under argon atmosphere. The resulting mixture was diluted with water (15 mL). The aqueous layer was extracted with DCM (2×15 mL). The resulting mixture was concentrated under vacuum. The residue was purified by silica gel column chromatography, eluted with CH₂Cl₂/MeOH (40:1). The product was dissolved in CH₂Cl₂ (20 mL), To the above mixture was added SiliaMetS thiol (250 mg). The resulting mixture was stirred overnight at room temperature. The resulting mixture was filtered, the filter cake was washed with CH₂Cl₂ (5 mL). The filtrate was concentrated under reduced pressure. This resulted in 65a (220 mg, 93.72%) as a yellow solid.

### Synthesis of 65

To a stirred solution of 65a (200.00 mg, 0.335 mmol, 1.00 equiv) in DCM (2.00 mL) was added TFA (0.40 mL). The resulting mixture was stirred for 1 h at room temperature. The resulting mixture was concentrated under vacuum. The residue was purified by Prep-TLC (CH₂Cl₂/MeOH 8:1) to afford 65 (150 mg, 90.12%) as a yellow solid.

LC-MS: (ES, m/z): [M+H]⁺ 497

H-NMR: (400 MHz, CD₃OD, ppm): δ 2.95 (s, 3H), 33.68 (s, 2H), δ 3.94-3.95 (d, 2H), δ 4.05-4.06 (d, 2H), δ 5.08 (s, 4H), δ 6.40 (s, 1H), δ 6.91-6.93 (d, 1H), δ 7.17 (s, 1H), δ 7.31 (s, 1H), δ 7.35 (s, 1H), δ 7.46-7.49 (m, 1H), δ 7.54 (s, 1H), δ 7.63-7.65 (m, 1H), δ 8.21 (s, 1H).

### Example 66. Synthesis of Compound 66

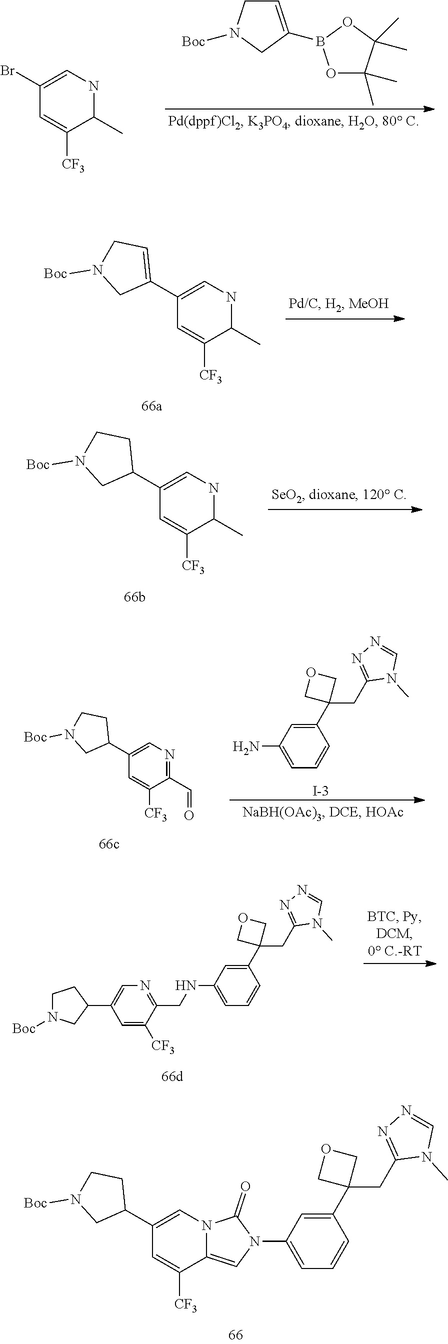

### Synthesis of 66a

Into a 20 mL round-bottom flask were added 5-bromo-2-methyl-3-(trifluoromethyl)pyridine (1.00 g, 4.166 mmol, 1.00 equiv), dioxane (8.00 mL), H₂O (2.00 mL), tert-butyl 3-(4,4,5,5-tetramethyl-1,3,2-dioxaborolan-2-yl)-2,5-dihydropyrrole-1-carboxylate (1.23 g, 4.167 mmol, 1.00 equiv), Pd(dppf)Cl₂ (0.30 g, 0.417 mmol, 0.1 equiv) and K₃PO₄ (1.77 g, 8.333 mmol, 2 equiv) at room temperature under nitrogen atmosphere. The resulting mixture was stirred for 5h at 80 degrees C. under nitrogen atmosphere. The resulting mixture was diluted with water (50 mL). The aqueous layer was extracted with EtOAc (3×30 mL). The resulting mixture was concentrated under vacuum. The residue was purified by Prep-TLC (PE/EtOAc 5:1) to afford 66a (1.1 g, 77.20%) as a white solid.

### Synthesis of 66b

To a stirred solution of 66a (1.00 g, 3.046 mmol, 1.00 equiv) in MeOH (30.00 mL) was added Pd/C (200.00 mg, 10%) at room temperature under nitrogen atmosphere. The mixture was hydrogenated at room temperature for 4 h under hydrogen atmosphere using a hydrogen balloon, filtered through a Celite pad and concentrated under reduced pressure. This resulted in 66b (1 g, 99.39%) as a white oil.

### Synthesis of 66c

To a stirred solution of 66b (1 g, 3,027 mmol, 1 equiv) in dioxane (10 mL) was added SeO₂ (1.01 g, 9.081 mmol, 3 equiv). The resulting mixture was stirred overnight at 110° C. The resulting mixture was diluted with water (50 mL). The resulting mixture was extracted with EtOAc (2×50 mL). The combined organic layers were dried over anhydrous Na₂SO₄. After filtration, the filtrate was concentrated under reduced pressure. This resulted in 66c (860 mg, 82.51%) as a yellow solid.

### Synthesis of 66d

To a stirred solution of 66c (350 mg, 1.016 mmol, 1 equiv) and 1-3 (248.32 mg, 1.016 mmol, 1 equiv) in DCE (10 mL) was added HOAc (122.08 mg, 2.032 mmol, 2 equiv) and NaBH(OAc)₃ (430.86 mg, 2.032 mmol, 2 equiv). The resulting mixture was stirred overnight at room temperature. The reaction was quenched by the addition of water (20 mL). The resulting mixture was extracted with CH₂Cl₂ (3×20 ml). The combined organic layers were concentrated under reduced pressure. The residue was purified by Prep-TLC (CH₂Cl₂/MeOH 20:1) to afford 66d (300 mg, 51.54%) as a light yellow solid.

### Synthesis of 66

To a stirred solution of 66d (300.00 mg, 0.524 mmol, 1.00 equiv) and Pyridine (248.64 mg, 3.144 mmol, 6.00 equiv) in DCM (5.00 mL) was added Triphosgene (54.41 mg, 0.183 mmol, 0.35 equiv) at 0 degrees C. The resulting mixture was stirred for 30 min at room temperature. The reaction was quenched by the addition of NaHCO₃ (aq.) (10 ml). The aqueous layer was extracted with CH₂Cl₂/MeOH=10/1 (3×20 mL). The resulting mixture was concentrated under vacuum. The crude product was re-crystallized from CH₂Cl₂/methyl tert-butyl ether (1:5) to afford Compound 66 (200 mg, 63.77%) as a yellow solid.

LC-MS: (ES, m/z): [M+H]⁺ 599

H-NMR: (400 MHz, DMSO-d6, ppm): δ 1.42 (s, 9H), δ 1.89-2.01 (m, 1H), δ2.02-2.08 (m, 1H), δ2.97 (s, 3H), δ3.14-3.16 (m, 1H), δ3.20-3.29 (m, 2H), δ3.42-3.46 (m, 1H), δ3.53 (s, 2H), δ3.60-3.65 (m, 1H), δ4.91-4.96 (m, 4H), δ6.89-6.91 (d, 1H), δ7.18-7.19 (d, 1H), δ7.32 (s, 1H), δ7.38-7.42 (m, 2H), δ7.61 (s, 1H), δ7.74-7.77 (m, 1H), δ8.20 (s, 1H).

### Example 67. Synthesis of Compound 67

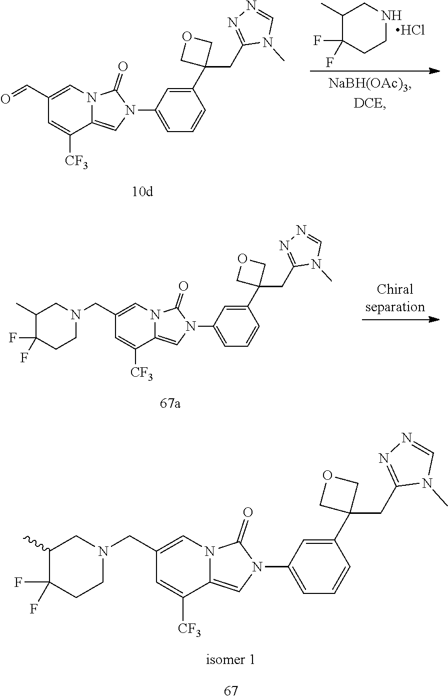

### Synthesis of 67a

Into a 100 mL round-bottom flask were added 10d (400 mg 0.874 mmol, 1.00 equiv), DCE (5.00 mL), 4,4-difluoro-3-methylpiperidine hydrochloride (149.45 mg, 0.874 mmol, 1 equiv) and NaBH(OAc)₃ (556.02 mg, 2.622 mmol, 3 equiv) at room temperature. The resulting mixture was stirred overnight at room temperature. The resulting mixture was concentrated under reduced pressure. The residue was purified by Prep-TLC eluted with CH₂Cl₂/MeOH (10:1) to afford 67a (100 mg, 19.83%) as a yellow solid.

### Synthesis of 67

The 67a (100 mg) was purified by chiral separation with the following conditions (Column: CHIRALPAK IC, 2*25 cm, 5 pnm; Mobile Phase A: Hex:DCM=3:1 (0.5% 2M NH₃-MeOH), Mobile Phase B: MeOH; Flow rate: 20 mL/min; Gradient: 50% B to 50% B in 18 min; Wave Length: 220/254 nm; RT1 (min): 14.18, RT2 (min): 16.17 the first peak is product) to afford Compound 67 (34.3 ng, 34.30%) as a yellow solid,

LC-MS: (ES, m/z): [M+H]⁺ 577

H-NMR: 1H NMR (300 MHz, DMSO-d6, ppm, δ) 0.92-0.94 (d, 3H), 1.99-2.15 (m, 4H), 2.21-2.32 (m, 1H), 2.62-2.81 (m, 2H), 2.97 (s, 3H), 3.32-3.35 (d, 2H), 3.53 (s, 2H), 4.91-4.96 (m, 4H), 6.89-6.91 (d, 1H), 7.03 (s, 1H), 7.32 (s, 1H), 7.38-7.42 (m, 2H), 7.71 (s, 1H), 7.74-7.76 (m, 1H), 8.20 (s, 1H).

### Example 68. Synthesis of Compound 68

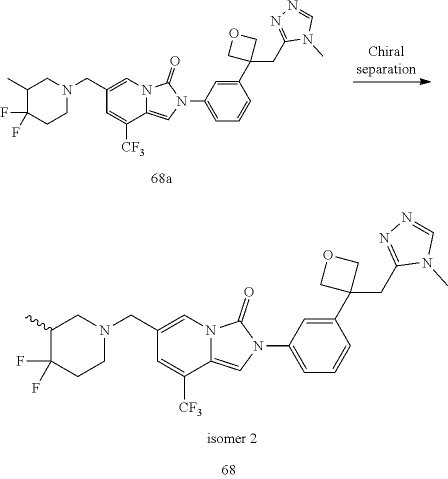 [SMILES: CC1CN(Cc2cc(C(F)(F)F)c3cn(-c4cccc(C5(Cc6nncn6C)COC5)c4)c(=O)n3c2)CCC1(F)F ; CC1CN(Cc2cc(C(F)(F)F)c3cn(-c4cccc(C5(Cc6nncn6C)COC5)c4)c(=O)n3c2)CCC1(F)F]

### Synthesis of 68

The 68a (100 mg) was purified by chiral separation with the following conditions (Column: CHIRALPAK IC, 2*25 cm, 5 μm; Mobile Phase A: Hex:DCM=3:1 (0.5% 2M NH₃-MeOH), Mobile Phase B: MeOH; Flow rate: 20 mL/min; Gradient: 50% B to 50% B in 18 min; Wave Length: 220/254 nm; RT1 (min): 14.18, RT2 (min): 16.17, the second peak is product) to afford Compound 68 (34.2 mg, 34.20%) as a yellow solid.

LC-MS: (ES, m/z): [M+H]⁺ 577

H-NMR: 1H NMR (300 MHz, DMSO-d6) δ 0.92-0.94 (d, 3H), 1.99-2.15 (m, 4H), 2.21-2.32 (m, 1H), 2.62-2.81 (m, 2H), 2.97 (s, 3H), 3.32-3.35 (d, 2H), 3.53 (s, 2H), 4.91-4.96 (m, 4H), 6.89-6.91 (d, 1H), 7.03 (s, 1H), 7.32 (s, 1H), 7.38-7.42 (m, 2H), 7.71 (s, 1H), 7.74-7.76 (m, 1H), 8.20 (s, 1H).

### Example 69. Synthesis of Compound 69

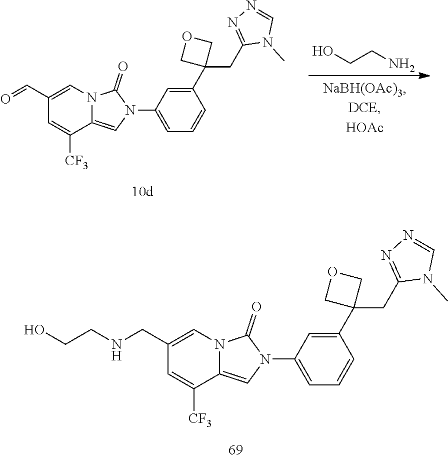

### Synthesis of 69

To a stirred solution of 10d (200.00 mg, 0,437 mmol, 1.00 equiv) and ethanolamine (40.06 mg, 0.656 mmol, 1.50 equiv) in DCE (3 mL) were added STAB (278.01 mg, 1.311 mmol, 3.00 equiv) and AcOH (26.26 mg, 0.437 mmol, 1.00 equiv) at room temperature. The resulting mixture was stirred overnight at room temperature. The reaction was quenched by the addition of sat. NH₄Cl (aq.) (10 mL) at room temperature. The resulting mixture was extracted with CH2Cl2 (3×10 mL). The resulting mixture was concentrated under reduced pressure. The crude product (101 mg) was purified by Prep-HPLC with the following conditions (Column: Xselect CSH F-Phenyl OBD column, 19*250 mm, 5 um; Mobile Phase A: Water (10 mmol/L NH₄HCO₃), Mobile Phase B: ACN; Flow rate: 25 mL/min; Gradient: 30% B to 47% B in 7 min; Wave Length: 220 nm; RT1 (min): 5.81) to afford Compound 69 (43.6 mg, 18.73%) as a yellow solid.

LC-MS: (ES, m/z): [M+H]⁺:503

H-NMR: (400 MHz, DMSO-d6, ppm): δ 2.61-2.64 (m, 2H), δ 2.90-2.91 (m, 3H), δ 3.30-3.32 (d, 2H), δ 3.47-3.57 (m, 4H), δ 4.91-4.96 (m, 4H), δ 6.88-6.90 (d, 1H), δ 7.14 (s, 1H), δ 7.38-7.40 (d, 1H), δ 7.41-7.42 (d, 2H), δ 7.42 (s, 1H), δ 7.43-7.44 (d, 1H), δ 8.20-8.21 (d, 2H).

### Example 70. Synthesis of Compound 70

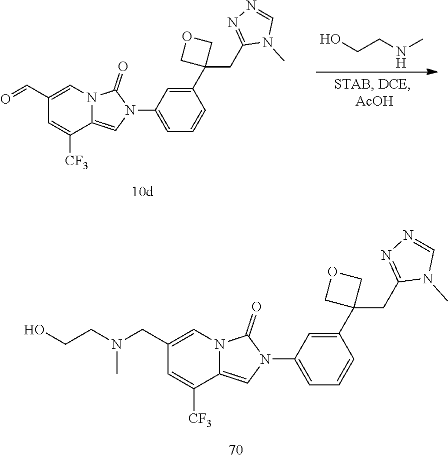

### Synthesis of 70

To a stirred solution of 10d (200.00 mg, 0,437 mmol, 1.00 equiv) and methylethanolamine (49.26 mg, 0,656 mmol, 1.50 equiv) in DCE (3 mL) were added STAB (278.01 mg, 1.311 mmol, 3.00 equiv) and AcOH (26.26 mg, 0.437 mmol, 1.00 equiv) at room temperature. The resulting mixture was stirred overnight at room temperature. The reaction was quenched by the addition of sat. NH₄Cl (aq.) (5 mL) at room temperature. The resulting mixture was extracted with CH₂Cl₂ (2×10 mL) The resulting mixture was concentrated under reduced pressure. The crude product (110 mg) was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 18% B to 28% B in 10 min; Wave Length: 220 nm; RT1 (min): 10.25) to afford 70 (42.3 mg, 18.62%) as a yellow solid.

LC-MS: (ES, m/z): [M+H]⁺:517

H-NMR: (400 MHz, DMSO-d6, ppm): δ 2.19 (m, 3H), δ 2.45-2.51 (m, 2H), δ 2.97 (s, 3H), δ 3.33 (s, 2H), δ 3.49-3.53 (m, 4H), δ 4.43-4.46 (m, 1H), δ 4.91-4.96 (m, 4H), δ 6.88-6.90 (d, 1H), δ 7.05 (s, 1H), δ 7.30 (s, 1H), δ 7.38-7.42 (m, 2H), δ 7.74 (s, 1H), δ 7.76-7.77 (d, 1H), δ 8.20 (s, 1H).

### Example 71. Synthesis of Compound 71

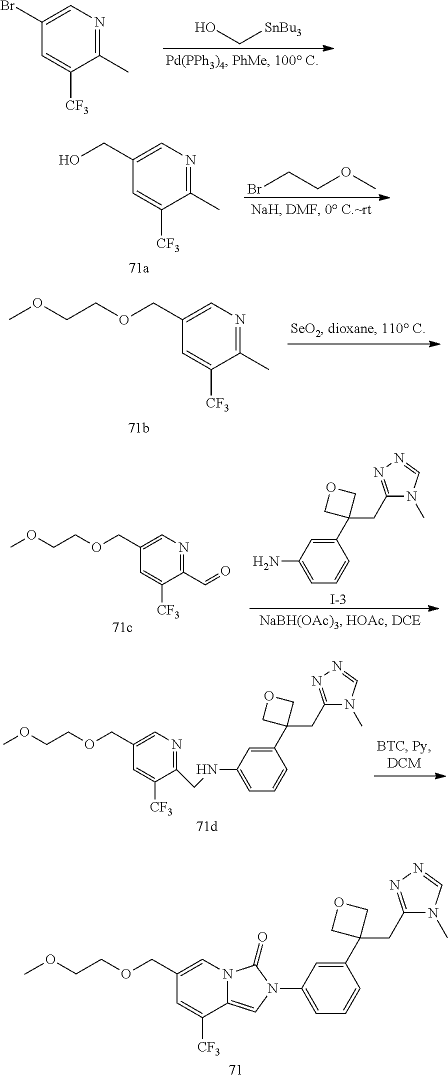

### Synthesis of 71a

To a stirred solution of 5-bromo-2-methyl-3-(trifluoromethyl)pyridine (3 g, 12.499 mmol, 1 equiv) and (tributylstannyl)methanol (6.02 g, 18.748 mmol, 1.5 equiv) in toluene (30 ml, 281.967 mmol, 22.56 equiv) was added Pd(PPh₃)₄ (0.29 g, 0.250 mmol, 0.02 equiv) at room temperature under nitrogen atmosphere. The resulting mixture was stirred for 4 h at 100° C. under nitrogen atmosphere. The resulting mixture was dilated with water (50 mL). The aqueous layer was extracted with EtOAc (2×20 mL). The resulting mixture was concentrated under reduced pressure. The residue was purified by silica gel column chromatography, elated with PE/EA=30:1 to afford 71a (1.3 g, 48.97%) as colorless oil.

### Synthesis of 71b

To a stirred mixture of 71a (380 mg, 0.988 mmol, 1 equiv) in DMF (5 mL) was added NaH (159.01 mg, 3,976 mmol, 2 equiv, 60%) at 0° C. under nitrogen atmosphere. The resulting mixture was stirred for 1 h at 0° C. under nitrogen atmosphere. To the above mixture was added 2-bromoethyl methyl ether (414.46 mug, 2.982 mmol, 1.5 equiv) at 0° C. The above mixture was stirred for 1 h at 0° C. under nitrogen atmosphere. The reaction was quenched by NH₄Cl(aq.) (30 mL). The aqueous layer was extracted with EtOAc (2×20 mL). The resulting mixture was concentrated under reduced pressure. The residue was purified by Prep-TLC (CH₂Cl₂/MeOH=100:1) to afford 71b (300 mg, 54.49%) as a colorless oil.

### Synthesis of 71b

To a stirred mixture of 71b (290 mg, 1.164 mmol, 1 equiv) in dioxane (4 mL) was added SeO₂ (516.44 mg, 4.656 mmol, 4 equiv) at room temperature. The resulting mixture was stirred for 4 h at 110° C. The resulting mixture was diluted with water (30 ml). The aqueous layer was extracted with EtOAc (2×10 mL). The resulting mixture was concentrated under reduced pressure. The residue was purified by Prep-TLC (CH₂Cl₂/MeOH=100:1) to afford 71c (180 mg, 52.31%) as a colorless oil.

### Synthesis of 71c

To a stirred mixture of 71c (170 mg, 0.646 mmol, 1 equiv) and I-3 (173.56 mg, 0.711 mmol, 1.1 equiv) in DCE (2 mL) were added HOAc (38.79 mg, 0.646 mmol, 1 equiv) and NaBH(OAc)₃ (410.65 mg, 1.938 mmol, 3 equiv) at room temperature. The resulting mixture was stirred overnight at room temperature. The reaction was quenched by water (30 mL). The aqueous layer was extracted with DCM (2×10 mL). The resulting mixture was concentrated under reduced pressure. The residue was purified by Prep-TLC (CH₂Cl₂/MeOH=30:1) to afford 71d (200 mg, 59.22%) as a yellow solid.

### Synthesis of 71

To a stirred solution of 71d (190 mg, 0.387 mmol, 1.00 equiv) and pyridine (607.52 mg, 2.322 mmol, 6 equiv) in DCM (2 mL) was added triphosgene (45.88 mg, 0.155 mmol, 0.4 equiv) at room temperature. The resulting mixture was stirred for 5 min at 0° C. The reaction was quenched by the addition of NaHCO₃ (aq.) (20 mL) at room temperature. The aqueous layer was extracted with CH₂Cl₂ (3×10 mL). The residue was purified by Prep-TLC (CH₂Cl₂/MeOH=15:1) to afford 71 (200 mg) as a yellow solid. The crude product (200 mg) was purified by Prep-HPLC with the following conditions (Column: XBridge Prep OBD C18 Column, 30*150 mm, 5 μm; Mobile Phase A: water (10 mmol/L NH₄CO₃), Mobile Phase B: ACN; Flow rate: 60 mL/min; Gradient: 30% B to 55% B in 8 min, Wave Length: 220 nm; RT1 (min): 7.23) to afford Compound 71 (30.7 mg, 15.35%) as a yellow solid.

LCMS: (ES, m/z): [M+H]⁺ 518.

H-NMR: (400 MHz, DMSO-d₆, ppm) δ 2.97 (s, 3H), 3.26 (s, 3H), 3.32-3.47 (m, 2H), 3.47-3.49 (m, 2H), 3.53-3.58 (m, 2H), 4.32 (s, 2H), 4.91-4.96 (m, 4H), 6.88-6.90 (d, 2H), 7.03 (s, 1H), 7.33-7.42 (m, 3H), 7.75-7.80 (m, 2H), 8.20 (s, 1H).

### Example 72. Synthesis of Compound 72

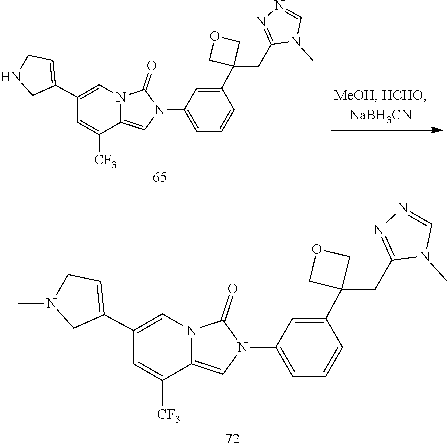

### Synthesis of 72

To a stirred solution of 65 (130.00 mg, 0.262 mmol, 1.00 equiv) and HCHO (63.75 mg, 0.786 mmol, 3 equiv, 37%) in MeOH (4.00 mL) were added HOAc (15.72 mg, 0.262 mmol, 1 equiv) and NaBH₃CN (110.99 mg, 0.524 mmol, 2 equiv). The resulting mixture was stirred for 2 h at room temperature. The reaction was quenched by the addition of water (20 mL). The resulting mixture was extracted with CH₂Cl₂/MeOH=10/1 (3×20 mL). The combined organic layers were concentrated under reduced pressure. The crude product was purified by Prep-HPLC with the following conditions (Column: Xselect CSH C18 OBD Column 30*150 mm 5 μm; Mobile Phase A: Water (0.1% FA), Mobile Phase B: ACN; Flow rate: 60 mL/min; Gradient: 10% B to 22% B in 7 min, Wave Length: 220 nm; RT1 (min): 6.62) to afford Compound 72 (49.7 mg, 37.18%) as a yellow solid.

LC-MS: (ES, m/z): [M+H]⁺ 511

H-NMR: (400 MHz, DMSO-d6, ppm): δ 2.33 (s, 3H), δ 2.97 (s, 3H), δ 3.51-3.56 (m, 4H), δ 3.71 (s, 2H), δ 4.88-4.96 (m, 4H), δ 6.45 (s, 1H), δ 6.88-6.90 (d, 1H), δ 7.39-7.43 (m, 5H), δ 7.74-7.76 (m, 1H), δ 8.21 (s, 1H).

### Example 73. Synthesis of Compound 73

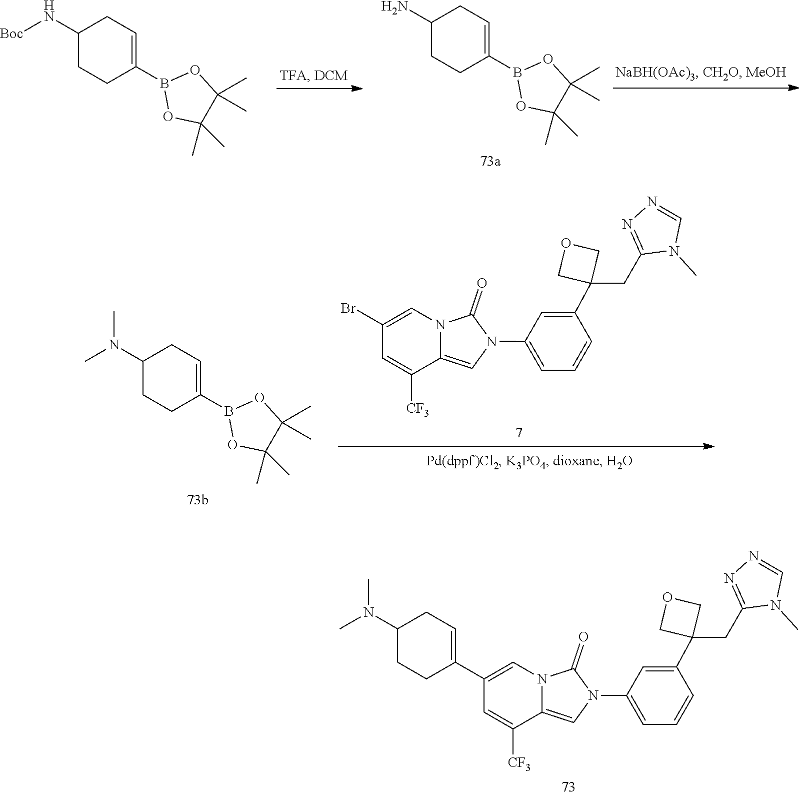

### Synthesis of 73a

To a stirred solution of tert-butyl N-[4-(4,4,5,5-tetramethyl-1,3,2-dioxaborolan-2-yl)cyclohex-3-en-1-yl]carbamate (1 g, 3.094 mmol, 1 equiv) in DCM (10 mL) was added TFA (2 mL). The resulting mixture was stirred for 3 h at room temperature. The resulting mixture was concentrated under reduced pressure. The residue was basified to pH 9 with saturated NaHCO₃ (aq.). The resulting mixture was extracted with CH₂Cl₂/MeOH=10/1 (3×20 mL). The combined organic layers were concentrated under reduced pressure. This resulted in 73a (600 mg, crude) as a light yellow solid.

### Synthesis of 73b

To a stirred solution of 73a (600 mg, 2.689 mmol, 1 equiv) and formaldehyde solution (872.91 mg, 10,756 mmol, 4 equiv, 37%) in MeOH (10 mL) were added NaBH₃CN (506.97 mg, 8.067 mmol, 3 equiv). The resulting mixture was stirred overnight at room temperature. The reaction was quenched by the addition of water (50 mL). The resulting mixture was extracted with CH₂Cl₂/MeOH=10/1 (4×50 mL). The combined organic layers were concentrated under reduced pressure. The residue was purified by silica gel column chromatography, elated with CH₂Cl₂/MeOH (30:1) to afford 73b (310 mg, 45.89%) as a light yellow solid.

### Synthesis of 73

To a stirred mixture of Compound 7 (100 mg, 0.197 mmol, 1 equiv) and 73b (98.83 mg, 0.394 mmol, 2 equiv) in dioxane (4 mL) and H₂O (1 mL) were added K₃PO (83.52 mg, 0.394 mmol, 2 equiv) and Pd(dppf)Cl₂ (14.40 mg, 0.020 mmol, 0.1 equiv). The resulting mixture was stirred overnight at 80° C. under argon atmosphere. The resulting mixture was diluted with water (10 mL). The resulting mixture was extracted with CH₂Cl₂/MeOH=10/1 (3×20 mL). The combined organic layers were concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: MeOH; Flow rate: 60 mL/min; Gradient: 40% B to 62% B in 8 min, Wave Length: 220 in; RT1 (min): 7.23) to afford Compound 73 (20.2 mg, 18.58%) as a yellow solid.

LC-MS: (ES, m/z): [M+H]⁺ 497

H-NMR: (400 MHz, DMSO, ppm): δ 1.45-1.46 (m, 1H), δ 1.99-2.02 (m, 1H), δ 2.10-2.15 (m, 1H), δ 2.22 (s, 6H), 32.31-2.36 (m, 3H), δ 2.45-2.49 (m, 1H), δ 2.97 (s, 3H), δ 3.54 (s, 2H), δ 4.88-4.96 (m, 4H), δ 6.28 (s, 1H), δ 6.89-6.90 (d, 1H), δ 7.33-7.42 (m, 4H), δ 7.54 (s, 1H), δ 7.75-7.77 (m, 1H), δ 8.22 (s, 1H).

### Example 74. Synthesis of Compound 74

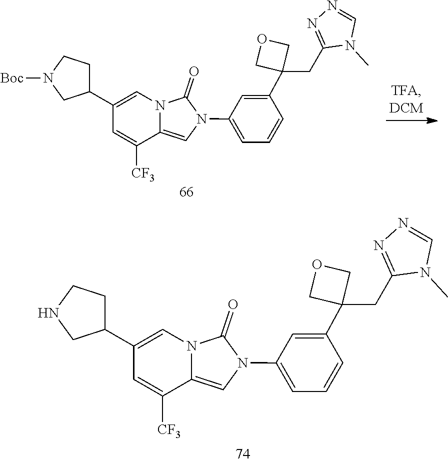 [SMILES: Cn1cnnc1CC1(c2cccc(-n3cc4c(C(F)(F)F)cc(C5CCN(C(=O)OC(C)(C)C)C5)cn4c3=O)c2)COC1 ; Cn1cnnc1CC1(c2cccc(-n3cc4c(C(F)(F)F)cc(C5CCNC5)cn4c3=O)c2)COC1]

### Synthesis of 74

To a stirred solution of 66 (200 mg, 0.334 mmol, 1 equiv) in DCM (2 mL) was added TFA (0.4 ml). The resulting mixture was stirred for 3 h at room temperature. The resulting mixture was concentrated under reduced pressure. The residue was purified by Prep-TLC (CH₂Clₕ/MeOH 10:1) to afford Compound 74 (140 mg, 84.06%) as a yellow solid.

LC-MS: (ES, m/z): [M+H]⁺ 599

H-NMR: (400 MHz, DMSO-d6, ppm): δ 1.66-1.71 (m, 1H), δ 2.18-2.12 (m, 1H), δ 2.73-2.77 (m, 1H), δ 2.89-2.93 (m, 1H), δ 2.98 (s, 3H), δ 3.14-3.20 (m, 3H), δ 3.53 (s, 2H), δ 4.93-4.97 (m, 4H), δ 6.90-6.91 (d, 1H), δ 7.13 (s, 1H), δ 7.32 (s, 1H), δ 7.39-7.43 (m, 2H), δ 7.61 (s, 1H), δ 7.75-7.77 (m, 1H), δ 8.21 (s, 1H).

### Example 75. Synthesis of Compound 75

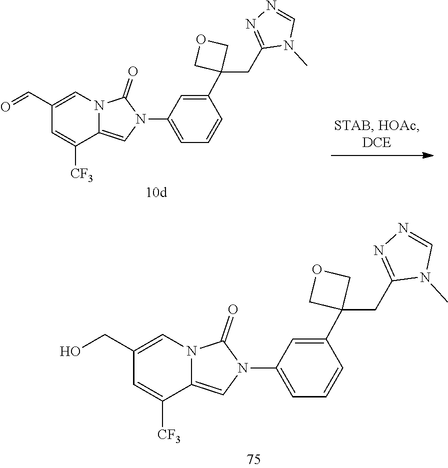 [SMILES: Cn1cnnc1CC1(c2cccc(-n3cc4c(C(F)(F)F)cc(C=O)cn4c3=O)c2)COC1 ; Cn1cnnc1CC1(c2cccc(-n3cc4c(C(F)(F)F)cc(CO)cn4c3=O)c2)COC1]

### Synthesis of 75

To a stirred solution of 10d (20.00 mg, 0.044 mmol, 1.00 equiv) and NaBH(OAc)₃ (27.80 mg, 0.131 mmol, 3.00 equiv) in DCE (1.00 mL) were added AcOH (2.63 mg, 0,044 mmol, 1 equiv) at room temperature. The resulting mixture was stirred overnight at room temperature. The residue was purified by Prep-TLC (CH₂Cl₂/MeOH 10:1) to afford Compound 75 (2.6 mg, 12.81%) as a yellow solid.

LC-MS: (ES, m/z): [M+H]⁺ 460

H-NMR: (400 MHz, CD₃OD, δ ppm): 2.96 (s, 3H), 3.66 (s, 2H), 4.44 (s, 2H), 5.06 (s, 4H), 6.88-6.90 (m, 1H), 7.07-7.12 (m, 2H), 7.32-7.33 (m, 1H), 7.47-7.49 (m, 1H), 7.62-7.65 (d, 1H), 7.72 (s, 1H), 8.21 (s, 1H).

### Example 76. Synthesis of Compound 76

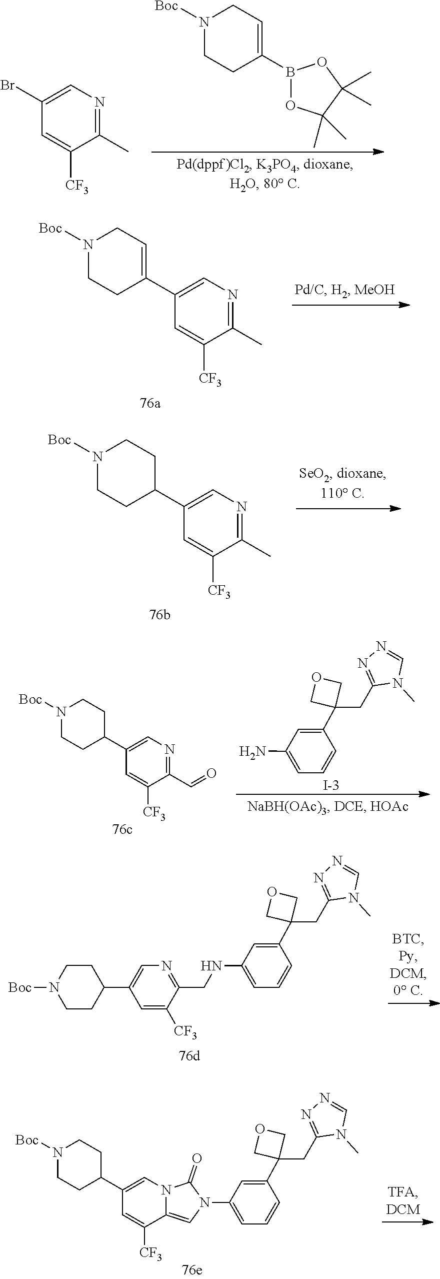

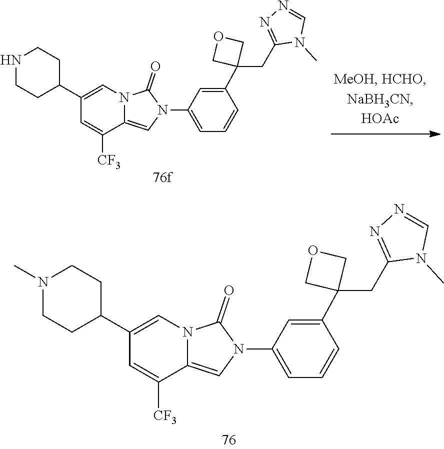

### Synthesis of 76a

Into a 20 mL sealed tube were added 5-bromo-2-methyl-3-(trifluoromethyl)pyridine (1.00 g, 4.166 mmol, 1.00 equiv), dioxane (8.00 mL), H₂O (2.00 mL), tert-butyl 4-(4,4,5,5-tetramethyl-1,3,2-dioxaborolan-2-yl)-3,6-dihydro-2H-pyridine-1-carboxylate (1.29 g, 4.172 mmol, 1.00 equiv), Pd(dppf)Cl₂ (0.30 g, 0.417 mmol, 0.10 equiv), and K₃PO₄ (1.77 g, 8.333 mmol, 2.00 equiv) at room temperature. The resulting mixture was stirred for 6h at 80 degrees C. under nitrogen atmosphere. The reaction was concentrated under reduced pressure. The residue was purified by Prep-TLC (PE/EtOAc 3:1) to afford 76a (1 g, 70.11%) as a white solid.

### Synthesis of 76b

To a solution of 76a (810 mg, 2.366 mmol, 1 equiv) in MeOH (20 mL) was added Pd/C (10%, 20 mg) under nitrogen atmosphere in a 100 mL round-bottom flask. The mixture was hydrogenated at room temperature for 4 h under hydrogen atmosphere using a hydrogen balloon, filtered through a Celite pad, and concentrated under reduced pressure. This resulted in 76b (820 mg) as a colorless oil.

### Synthesis of 76c

To a stirred solution of 76b (820 mg, 2.381 mmol, 1 equiv) in dioxane (20 mL) was added SeO₂ (792.62 mg, 7,143 mmol, 3 equiv). The resulting mixture was stirred overnight at 110° C. The resulting mixture was diluted with water (80 mL). The resulting mixture was extracted with EtOAc (2×50 mL). The combined organic layers were dried over anhydrous Na₂SO₄, After filtration, the filtrate was concentrated under reduced pressure. This resulted in 76c (760 mg, 89.07%) as a yellow oil.

### Synthesis of 76d

To a stirred mixture of 76c (350 mg, 0.977 mmol, 1 equiv) and I-3 (238.60 mg, 0.977 mmol, 1 equiv) in DCE (10 mL) was added HOAc (58.65 mg, 0.977 mmol, 1 equiv) and NaBH(OAc)3 (413.99 mg, 1.954 mmol, 2 equiv). The resulting mixture was stirred overnight at room temperature. The resulting mixture was diluted with water (10 mL). The resulting mixture was extracted with CH₂Cl₂/MeOH (2×10 mL). The combined organic layers were concentrated under reduced pressure. The residue was purified by Prep-TLC (CH₂Cl₂/MeOH 15:1) to afford 76d (360 mg, 62.83%) as a off-white solid.

### Synthesis of 76e

To a stirred solution of 76d (360 mg, 0.614 mmol, 1 equiv) and Pyridine (291.23 mg, 3.684 mmol, 6 equiv) in DCM (10 mL) was added Triphosgene (72.84 mg, 0.246 mmol, 0.4 equiv) at 0° C. The resulting mixture was stirred for 1h at 0° C. The reaction was quenched by the addition of NaHCO₃ (aq.) (10 mL). The resulting mixture was extracted with CH₂Cl₂/MeOH=10/1 (3×10 mL). The combined organic layers were concentrated under reduced pressure. The residue was purified by Prep-TLC (CH₂Cl₂/MeOH 20:1) to afford 76e (220 mg, 58.52%) as a yellow solid.

### Synthesis of 76f

To a stirred solution of 76e (320 mg, 0.522 mmol, 1 equiv) in DCM (2 mL) was added TFA (0.5 mL). The resulting mixture was stirred for 3 h at room temperature. The resulting mixture was concentrated under vacuum. The residue was purified by Prep-TLC (CH₂Cl₂/MeOH 10:1) to afford 76f (170 mg, 63.50%) as a yellow solid.

### Synthesis of 76

To a stirred solution of 76f (150 mg, 0,293 mmol, 1 equiv) and formaldehyde solution (71.25 mg, 0.879 mmol, 3 equiv, 37%) in MeOH (5 mL) were added HOAc (17.57 mg, 0,293 mmol, 1 equiv) and NaBH₃CN (36.78 mg, 0.586 mmol, 2 equiv). The resulting mixture was stirred for 2 h at room temperature. The reaction was quenched by the addition of NaHCO₃ (aq.) (20 mL). The resulting mixture was extracted with CH₂Cl₂/MeOH=10/1 (3×20 mL). The combined organic layers were concentrated under reduced pressure. The residue was purified by Prep-TLC (CH₂Cl₂/MeOH 10:1) to afford Compound 76 (107 mg, 69.43%) as a yellow solid.

LC-MS: (ES, m/z): [M+H]⁺ 527

H-NMR: (400 MHz, DMSO-d6, ppm): δ 1.62-1.66 (m, 2H), δ 1.68-1.79 (m, 2H), δ 1.92-1.98 (m, 2H), δ 2.21 (s, 3H), δ 2.34-2.40 (m, 1H), δ 2.83-2.89 (m, 2H), δ 3.02 (s, 3H), δ 3.59 (s, 2H), δ 4.91-4.96 (m, 4H), δ 6.89-6.91 (d, 1H), δ 7.10 (s, 1H), δ 7.29 (s, 1H), δ 7.38-7.42 (m, 2H), δ 7.47 (s, 1H), δ 7.74-7.76 (d, 1H), δ 8.20 (s, 1H).

### Example 77. Synthesis of Compound 77

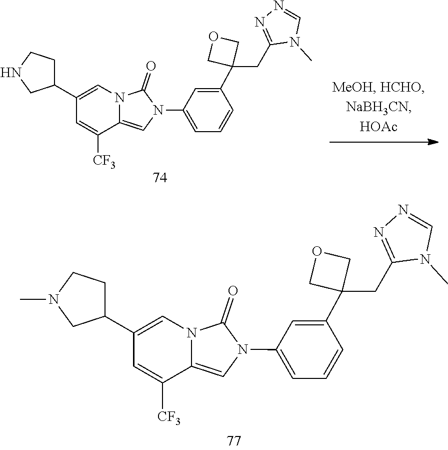

### Synthesis of 77

To a stirred solution of 74 (130 mg, 0.261 mmol, 1 equiv) and HCHO (63.49 mag, 0.783 mmol, 3 equiv, 37%) in MeOH (2 mL) were added HOAc (15.66 mg 0261 mmol, 1 equiv) and NaBH₃CN (32.78 mg, 0.522 mmol, 2 equiv). The resulting mixture was stirred for 2 h at room temperature. The reaction was quenched by the addition of water (15 mL). The resulting mixture was extracted with CH₂Cl₂/MeOH=10/1 (3×20 mL). The combined organic layers were concentrated under reduced pressure. The residue was purified by Prep-TLC (CH₂Cl₂/MeOH 10:1) to afford 77 (48.2 mg, 36.06%) as a yellow solid.

LC-MS: (ES, m/z): [M+H]⁺ 513

H-NMR: (400 MHz, DMSO-d6, ppm): δ 1.65-1.72 (m, 1H), δ 2.14-2.19 (m, 1H), δ 2.20 (s, 3H), δ 2.31-2.45 (m, 2H), δ 2.63-2.67 (m, 1H), δ 2.72-2.76 (m, 1H), δ 2.97 (s, 3H), δ 3.23-3.29 (m, 1H), δ 3.54 (s, 3H), δ 4.91-4.96 (m, 4H), 56.88-6.91 (d, 1H), δ 7.12 (s, 1H), δ 7.31 (s, 1H), δ 7.38-7.42 (m, 2H), δ 7.74-7.77 (d, 1H), δ 8.20 (s, 1H).

### Example 78. Synthesis of Compound 78

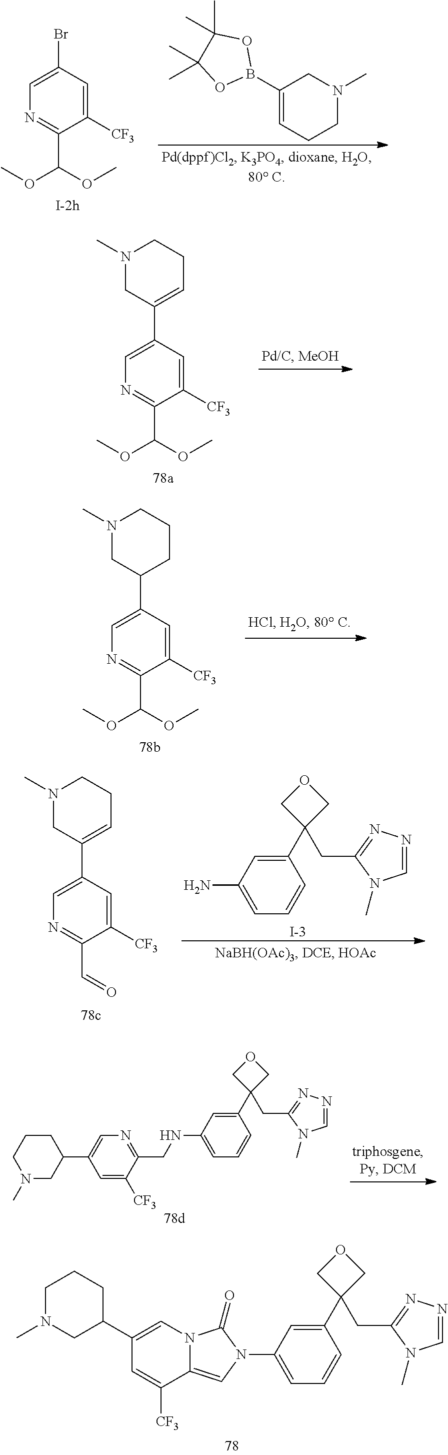

### Synthesis of 78a

To a stirred mixture of 1-2h (500.00 mg, 1.666 mmol, 1.00 equiv) and 1-methyl-3-(4,4,5,5-tetramethyl-1,3,2-dioxaborolan-2-yl)-5,6-dihydro-2H-pyridine (446.13 mg, 2.000 mmol, 1.20 equiv) in dioxane (4.00 mL) and H₂O (1.00 mL) were added K₃PO₄ (707.38 mg, 3,333 mmol, 2.00 equiv) and Pd(dppf)C12 (243.84 mg, 0,333 mmol, 0.20 equiv) at room temperature under nitrogen atmosphere. The resulting mixture was stirred overnight at 80 degrees C. under nitrogen atmosphere. The reaction was quenched with water (100 mL) at room temperature. The aqueous layer was extracted with EtOAc (3×50 mL). The resulting mixture was concentrated under reduced pressure. The residue was purified by silica gel column chromatography, eluted with PE/EtOAc (5:1) to afford 78a (400 mg, 75.89%) as a brown yellow oil.

### Synthesis of 78b

To a solution of 78a (400.00 mg, 1.265 mmol, 1.00 equiv) in MeOH (5.00 mL) was added Pd/C (79.40 mg, 0.746 mmol, 0.59 equiv) in a pressure tank. The mixture was hydrogenated at room temperature overnight under hydrogen atmosphere using a hydrogen balloon, filtered through a Celite pad, and concentrated under reduced pressure. This resulted in 78b (300 mg, 74.53%) as colorless oil.

### Synthesis of 78c

Into a 50 mL 3-necked round-bottom flask were added 78b (300.00 mg, 0.942 mmol, 1.00 equiv) and H₂O (6.00 mL) and HCl (0.50 mL) at room temperature. The resulting mixture was stirred overnight at 80 degrees C. under nitrogen atmosphere. The reaction was quenched by the addition of NaHCO₃ (aq.) (100 mL) at room temperature. The aqueous layer was extracted with EtOAc (3×30 mL). The resulting mixture was concentrated under reduced pressure. The residue was purified by Prep-TLC (CH₂Cl₂/MeOH 10:1) to afford 78c (200 mg, 77.95%) as colorless oil.

### Synthesis of 78d

To a stirred mixture of 78c (180.00 mg, 0.661 mmol 1.00 equiv) and 13 (193.81 mg, 0.793 mmol, 1.20 equiv) in DCE. (2.00 mL) were added HOAc (39.70 mg, 0,661 mmol, 1.00 equiv) and NaBH(OAc)₃ (280.23 mg, 1.322 mmol, 2.00 equiv) at room temperature. The resulting mixture was stirred overnight at room temperature. The reaction was quenched with sat. NH₄Cl (aq.) (100 mL) at room temperature. The aqueous layer was extracted with EtOAc (3×30 mL). The resulting mixture was concentrated under reduced pressure. The residue was purified by Prep-TLC (PE/EtOAc 5:1) to afford 78d (150 mg, 45.33%) as colorless oil,

### Synthesis of 78

To a stirred mixture of 78d (200.00 mg, 0.400 mmol, 1.00 equiv) and Pyridine (191.99 mg, 2.397 mmol, 6.00 equiv) in DCM (8.00 mL) was added triphosgene (15.26 mg, 0.140 mmol, 0.35 equiv) in portions at room temperature under nitrogen atmosphere. The resulting mixture was stirred for 30 min at room temperature under nitrogen atmosphere. The reaction was quenched with NaHCO₃ (aq.) (100 mL) at room temperature. The precipitated solids were collected by filtration and washed with DCM (3×20 mL). The resulting mixture was concentrated under reduced pressure. The residue was purified by Prep-TLC (CH₂Cl₂/MeOH 5:1) to afford Compound 78 (52.3 mg, 24.86%) as a yellow solid

LC-MS: (ES, m/z): [M+H]⁺ 527

H-NMR: (400 MHz, DMSO, δ ppm): 1.32-1.40 (m, 1H), 1.50-1.59 (m, 1H), 1.66-1.70 (m, 1H), 1.76-1.79 (m, 1H), 1.97-2.00 (d, 2H), 2.20 (s, 3H), 2.63-2.68 (m, 2H), 2.77-2.80 (d, 1H), 2.97-3.01 (d, 3H), 3.53 (s, 2H), 4.87-4.96 (m, 4H), 6.88-6.90 (d, 1H), 7.11 (s, 1H), 7.28-7.38 (d, 1H), 7.40-7.42 (t, 2H), 7.58 (s, 1H), 7.73-7.76 (m, 1H), 8.19-8.22 (d, 1H).

### Example 79. Synthesis of Compound 79

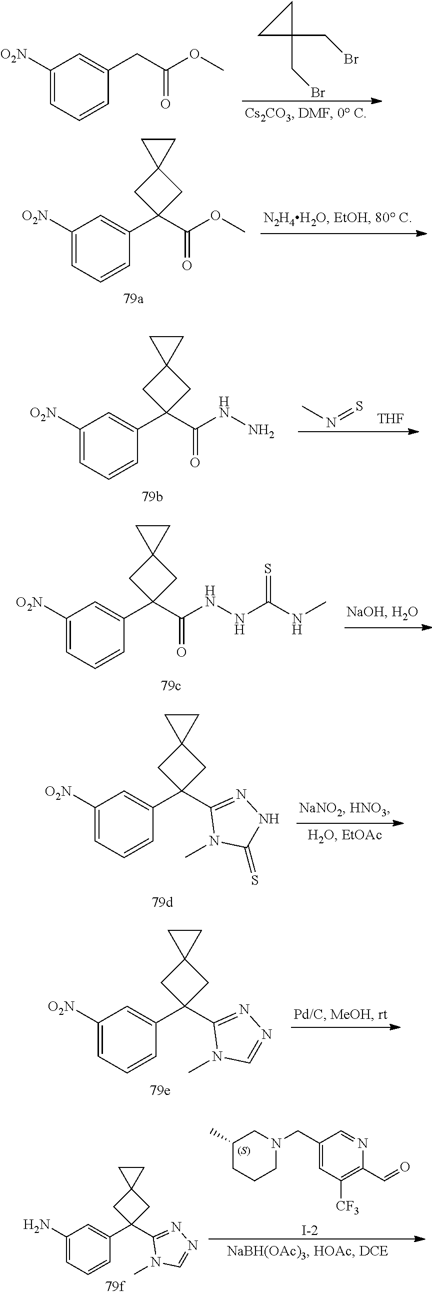

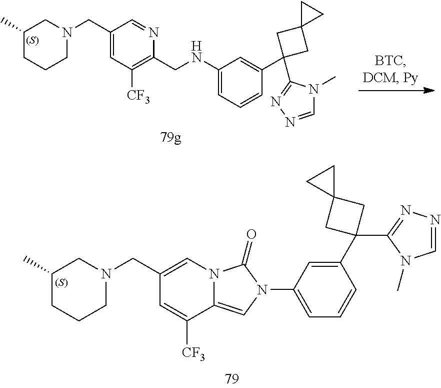

### Synthesis of 79a

To a stirred solution of methyl 2-(3-nitrophenyl)acetate (3.5 g, 17.933 mmol, 1 equiv) in DMF (70 mL) was added Cs₂CO₃ (11.69 g, 35.866 mmol, 2 equiv) at 0° C. under air atmosphere. The resulting mixture was stirred for 2h at 0 degrees C. under air atmosphere. To the above mixture was added 1,1-bis(bromomethyl)cyclopropane (12.26 g, 53.799 mmol, 3 equiv) at 0° C. The resulting mixture was stirred overnight at room temperature. The reaction was quenched with water at room temperature. The aqueous layer was extracted with EtOAc (2×200 mL). The resulting mixture was concentrated under reduced pressure. The residue was purified by silica gel column chromatography, eluted with PE/EtOAc (50:1) to afford 79a (560 mg, 11.00%) as a colorless oil.

### Synthesis of 79b

To a stirred solution of 79a (560 mg, 2.143 mmol, 1 equiv) in EtOH (6 mL, 103,281 mmol, 48.19 equiv) was added hydrazine hydrate (98%) (1071.5 mg, 21.43 mmol, 10 equiv) at room temperature. The resulting mixture was stirred overnight at 80° C. The reaction was quenched with water (40.00 mL) at room temperature. The aqueous layer was extracted with (CH₂Cl₂/MeOH 10:1) (2×80 mL). The resulting mixture was concentrated under vacuum. The residue was purified by silica gel column chromatography, eluted with CH₂Cl₂/MeOH (20:1) to afford 79b (480 mg, 81.43%) as a colorless oil.

### Synthesis of 79c

To a stirred solution of 79b (480 mg, 1,837 mmol, 1 equiv) in tetrahydrofuran (6 ML) was added methyl isothiocyanate (265.93 mg, 3.637 mmol, 1.98 equiv) at room temperature. The resulting mixture was stirred for 2h at room temperature. The resulting mixture was diluted with water (30 mL). The resulting mixture was concentrated under reduced pressure. The precipitated solids were collected by filtration and washed with water (2×2 mL). This resulted in 79c (510 mg, 80.53%) as a white solid.

### Synthesis of 79d

To a stirred mixture of NaOH (122.00 mg, 3.050 mmol, 2 equiv) in H₂O (6 mL) was added 79c (510 mg, 1.525 mmol, 1 equiv) at room temperature. The resulting mixture was stirred overnight at room temperature. The mixture was acidified to pH 4 with HCl (aq.). The resulting mixture was filtered and the filter cake was washed with water (3×1 mL). The filtrate was concentrated under reduced pressure. This resulted in 79d (480 mg, 94.50%) as a white solid.

### Synthesis of 79e

To a stirred solution of 79d (250 mg, 0.790 mmol, 1.00 equiv) and NaNO₂ (545.19 mg, 7.900 mmol, 10 equiv) in ethyl acetate (13 mL) and 1120 (3 mL) were added HNO₃ (497.92 mg, 7.900 mmol, 10 equiv) at 0° C. The resulting mixture was stirred overnight at room temperature. The mixture was acidified to pH 7 with saturated NaHCO₃ (aq.). The aqueous layer was extracted with CH₂Cl₂ (3×7 mL). The resulting mixture was concentrated under vacuum. The residue was purified by Prep-TLC (PE/EA 2:1) to afford 79e (180 mg, 74.51%) as a white solid.

### Synthesis of 79f

To a stirred solution of 79e (150 mg, 0.528 mmol, 1 equiv) in MeOH (4 mL) was added Pd/C (45 mg, 10%) at room temperature under nitrogen atmosphere. The resulting mixture was stirred for 5h at room temperature under hydrogen atmosphere. The resulting mixture was filtered through a celite pad and the filter cake was washed with MeOH (3×5 mL). The filtrate was concentrated under reduced pressure. The resulting mixture was concentrated under vacuum. The residue was purified by Prep-TLC (DCM/MeOH 12:1) to afford 79f (110 mg, 73.78%) as a white solid.

### Synthesis of 79g

To a stirred solution of 79f (80 mg, 0.315 mmol, 1 equiv) and I-2 (90.05 mg, 0.315 mmol, 1 equiv) in DCE (2 mL) were added NaBH(OAc)₃ (199.99 mg, 0.945 mmol, 3 equiv) and HOAc (37.78 mg, 0.630 mmol, 2 equiv) at room temperature. The reaction was quenched with NH₄Cl (aq.) at room temperature. The resulting mixture was stirred for 2h at 0° C. The aqueous layer was extracted with EtOAc (3×5 mL). The resulting mixture was concentrated under vacuum. The residue was purified by Prep-TLC (CH₂Cl₂/MeOH 10:1) to afford 79g (69 mg, 36.80%) as a white solid.

### Synthesis of 79

To a stirred solution of 79g (60.00 mg, 0.114 mmol, 1.00 equiv) and Pyridine (54.28 mg, 0.686 mmol, 6 equiv) in DCM (1.50 mL) was added Triphosgene (30.54 mg, 0.103 mmol 0.90 equiv) at 0° C. The resulting mixture was stirred for 30 min at 0° C. The reaction was quenched with NaHCO₃ (aq.) (3.00 mL) at room temperature. The aqueous layer was extracted with DCM (3×8 mL) The resulting mixture was concentrated under vacuum. The residue was purified by reverse flash chromatography with the following conditions: column, C18 silica gel; mobile phase, A: water (0.5% NH₄HCO₃), B: CH₃CN, 30% B to 50% B gradient in 30 min; detector, UV 254 nm. This resulted in Compound 79 (14.8 mg, 23.22%) as a yellow solid.

LC-MS: (ES, m/z): [M+H]⁺ 551

H-NMR: (400 MHz, DMSO-d₆, ppm, δ): 0.42-0.47 (m, 2H), 0.53-0.62 (m, 2H), 1.44-1.50 (m, 1H), 1.58-1.67 (m, 4H), 1.86-1.92 (m, 1H), 2.68-2.79 (d, 4H), 3.18-3.33 (t, 7H), 7.01 (s, 1H), 7.18-7.20 (d, 1H), 7.36 (s, 1H), 7.49-7.53 (m, 1H), 7.63-7.67 (d, 2H), 7.84 (s, 1H), 8.40 (s, 1H).

### Example 80. Synthesis of Compound 80

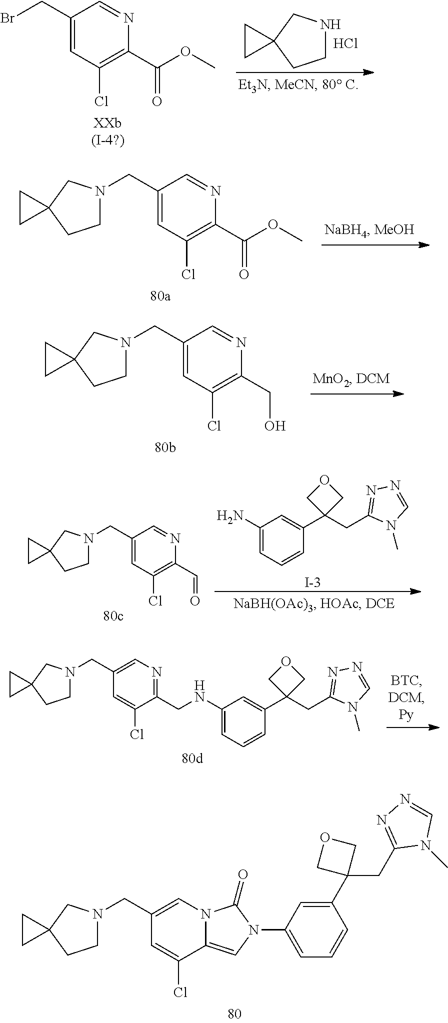

### Synthesis of 80a

To a stirred solution of 61b (950.00 mg, 3.592 mmol, 1.00 equiv) and 5-azaspiro[2,4]heptane hydrochloride (955.34 mg, 7.183 mmol, 2 equiv) in acetonitrile (10.00 mL) were added Et₃N (725.48 mg, 7.183 mmol, 2 equiv) at room temperature. The resulting mixture was stirred overnight at 80 degrees C. under nitrogen atmosphere. The reaction was quenched with water at room temperature. The aqueous layer was extracted with EtOAc (3×50 mL). The resulting mixture was concentrated under reduced pressure. The residue was purified by silica gel column chromatography, eluted with PE/EtOAc (5:1) to afford 80a (700 mg, 65.95%) as an off-white solid.

### Synthesis of 80b

To a stirred solution of 80a (700.00 mg, 2.493 mmol, 1.00 equiv) in MeOH (8.00 mL) was added NaBH₄ (188.66 mg, 4.987 mmol, 2 equiv) at 0 degrees C. The resulting mixture was stirred for 5 h at room temperature under air atmosphere. The reaction was quenched with NH₄Cl (aq.) at room temperature. The aqueous layer was extracted with EtOAc (3×20 mL). The resulting mixture was concentrated under reduced pressure. The residue was purified by Prep-TLC (CH-2C4/MeOH 20:1) to afford 80b (310 mg, 47.72%) as a white oil.

### Synthesis of 80c

To a stirred solution of 80b (290.00 mg, 1.147 mmol, 1.00 equiv) in DCM (3.00 mL) was added MnO₂ (997.53 mg, 11.474 mmol, 10 equiv) at room temperature. The resulting mixture was stirred overnight at 40 degrees C. The resulting mixture was filtered and the filter cake was washed with DCM (3×10 ml). The filtrate was concentrated under reduced pressure. The residue was purified by Prep-TLC (PE/EA 2:1) to afford 80c (310 mg, 96.98%) as a white oil.

### Synthesis of 80d

To a stirred solution of 80c (300.00 mg, 1.197 mmol, 1.00 equiv) in DCE (3.00 mL) were added 1-3 (292.30 mg, 1.197 mmol, 1 equiv) at room temperature. The resulting mixture was stirred for 1h at room temperature. To the above mixture was added NaBH(OAc)3 (760.77 mg, 3.590 mmol, 3.00 equiv) and HOAc (7.19 mg, 0.120 mmol, 0.1 equiv) at room temperature. The resulting mixture was stirred for an additional 2h at room temperature. The reaction was quenched with NH₄Cl (aq.) at room temperature. The aqueous layer was extracted with DCM (3×20 mL). The resulting mixture was concentrated under reduced pressure. The residue was purified by Prep-TLC (CH₂Cl₂/MeOH=20:1) to afford 80d (140 rug, 14.66%) as an off-white oil,

### Synthesis of 80

To a stirred solution of 80d (140.00 mg, 0,292 mmol, 1.00 equiv) and Pyridine (138.71 mg, 1.754 mmol, 6 equiv) in DCM (2.00 mL) was added Triphosgene (30.35 mg, 0.102 mmol, 0.35 equiv) at 0 degrees C. The resulting mixture was stirred for 30 min at 0 degrees C. The reaction was quenched with NaHCO₃ (aq.) (3.00 mL) at room temperature. The aqueous layer was extracted with DCM (3×8 mL). The resulting mixture was concentrated under vacuum. The residue was purified by reverse flash chromatography with the following conditions: column, C18 silica gel; mobile phase, A: water (0.5% NH₄CO₃), B: CH₃CN, 30% B to 50% B gradient in 30 min; detector, UV 254 nm. This resulted in Compound 80 (4.8 mg 3.20%) as a yellow solid.

LC-MS: (ES, m/z): [M+H]⁺ 505

H-NMR: (400 MHz, DMSO-d6, ppm): δ 0.60 (s, 1H), δ 1.84-1.91 (m, 1H), δ 2.05-2.06 (m, 1H), δ 3.06 (s, 3H), δ 3.23 (s, 2H), δ 3.32-3.33 (m, 1H), δ 0.68-0.74 (m, 3H), δ 3.60 (s, 3H), δ 4.16-4.26 (m, 2H), δ 4.93 (s, 4H), δ 6.88 (s, 1H), δ 6.97-6.99 (d, 1H), δ 7.41-7.78 (m, 4H), δ 7.93 (s, 1H), δ 8.47 (s, 1H).

### Example 81. Synthesis of Compound 81

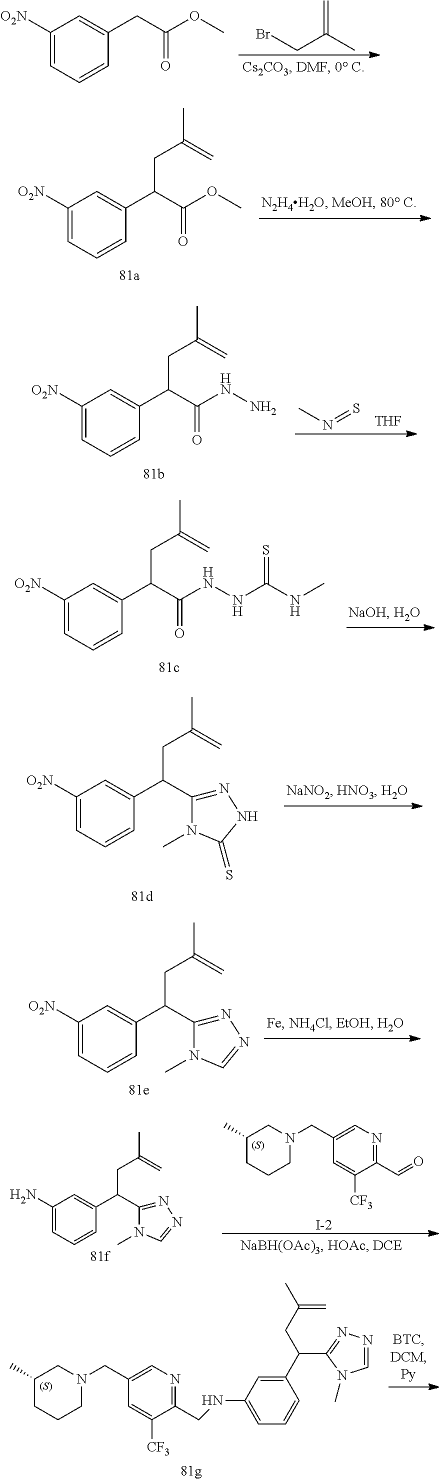

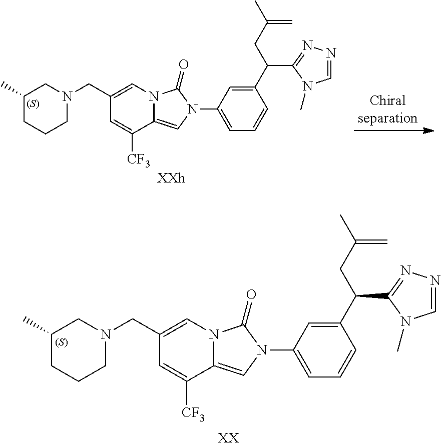

### Synthesis of 81a

Into a 250 mL 3-necked round-bottom flask were added methyl 2-(3-nitrophenyl)acetate (5 g, 25.618 mmol, 1 equiv) and DMF (50 mL) at room temperature. To the above mixture was added Cs₂CO₃ (25.04 g, 76.854 mmol, 3 equiv) at 0° C. The mixture was stirred for 3h at 0° C., To the above mixture was added 3-bromo-2-methylprop-1-ene (6.92 g, 51.236 mmol, 2 equiv) at room temperature. The resulting mixture was stirred overnight at rosin temperature. The reaction was quenched with water (150 mL) at room temperature. The aqueous layer was extracted with EtOAc (3×50 mL). The resulting mixture was concentrated under reduced pressure. The residue was purified by silica gel column chromatography, eluted with PE/EA (50:1) to afford 81a (3.4 g, 53.24%) as a white solid.

### Synthesis of 81b

A solution of 81a (3.4 g, 13.640 mmol, 1 equiv) and hydrazine hydrate (10.93 g, 218.240 mmol, 16 equiv) in MeOH (17 mL) was stirred for 2h at 80° C. under nitrogen atmosphere. The reaction was quenched by the addition of water (50 mL) at room temperature. The resulting mixture was concentrated under reduced pressure. The precipitated solids were collected by filtration and washed with MeCN (2×30 mL). This resulted in 81b (3 g, 88.23%) as a white solid.

### Synthesis of 81c

To a stirred solution of 81b (3 g, 12.035 mmol, 1 equiv) in tetrahydrofuran (30 mL) was added methyl isothiocyanate (1.76 g, 24.070 mmol, 2 equiv) at room temperature. The resulting mixture was stirred overnight at room temperature. The reaction was quenched by the addition of water (50 mL) at room temperature. The resulting mixture was concentrated under reduced pressure. The precipitated solids were collected by filtration and washed with MeCN (2×20 mL). This resulted in 81c (2.98 g, 76.81%) as a white solid.

### Synthesis of 81d

Into a 100 ml, round-bottom flask were added H₂O (40 mL) and NaOH (1 g, 25,002 mmol, 2.69 equiv) at room temperature. To the above mixture was added 81c (3 g, 9.306 mmol, 1 equiv) at room temperature. The resulting mixture was stirred overnight at room temperature. The residue was acidified to pH 5 with 1 mol/L HCl (aq.). The resulting mixture was filtered, the filter cake was washed with water (3×20 mL). The resulting solid was dried under vacuum. This resulted in 81d (2.5 g, 80.32%) as a yellow solid.

### Synthesis of 81e

Into a 100 mL round-bottom flask were added H₂O (25 ml), NaNO₂ (2.83 g, 41.070 mmol, 5 equiv) and 81d (2.5 g, 8.214 mmol, 1 equiv) at room temperature. To the above mixture was added HNO₃ (25 mL, 1 mol/L) dropwise at 0° C. The resulting mixture was stirred for 5 b at 0° C. The reaction was quenched with NaHCO₃ (aq.) (50 mL) at room temperature. The aqueous layer was extracted with EtOAc (3×40 mL). The resulting mixture was concentrated under vacuum. The residue was purified by silica gel column chromatography, eluted with DCM/MeOH (100:1) to afford 81e (2 g, 80.48%) as a yellow oil.

### Synthesis of 81f

Into a 100 mL 3-necked round-bottom flask were added 81e (2 g, 7.345 mmol, 1 equiv), Fe (2.05 g, 36.725 mmol, 5 equiv), NH₄Cl (1.96 g, 36.725 mmol, 5 equiv), EtOH (30 mL) and H₂O (10 mL) at room temperature. The resulting mixture was stirred overnight at 95° C. The resulting mixture was filtered, the filter cake was washed with DCM/MeOH (20:1) (3×20 mL). The filtrate was concentrated under vacuum. The residue was purified by Prep-TLC (DCM/MeOH 15:1) to afford 81f (800 mg, 42.70%) as a white solid.

### Synthesis of 81g

Into a 50 mL round-bottom flask were added 81f (400 mg, 1,651 mmol, 1 equiv), 1-2 (519.84 mg, 1.816 mmol, 1.1 equiv) and DCE (10 mL) at room temperature. To the above mixture was added STAB (699.69 mg, 3.302 mmol, 2 equiv) at room temperature. The resulting mixture was stirred overnight at room temperature. The reaction was quenched with NH₄Cl (aq.) (50 mL) at room temperature. The aqueous layer was extracted with EtOAc (3×20 mL). The resulting mixture was concentrated under vacuum. The residue was purified by Prep-TLC (DCM/MeOH 15:1) to afford 81g (300 mg, 33.33%) as a Brown yellow solid.

### Synthesis of 81h

Into a 50 mL round-bottom flask were added 81g (280 mg, 0,546 mmol, 1 equiv), DCM (10 mL) and pyridine (345.64 mg, 4.368 mmol, 8 equiv) at room temperature. To the above mixture was added Triphosgene (113.46 mg, 0.382 mmol, 0.7 equiv) at room temperature. The resulting mixture was stirred for 5 min at room temperature. The reaction was quenched with NaHCO₃ (aq.) (40 mL) at room temperature. The aqueous layer was extracted with EtOAc (3×20 mL). The resulting mixture was concentrated under vacuum. The residue was purified by Prep-TLC (DCM/MeOH 15:1) to afford 81h (150 mg, 47.42%) as a yellow solid,

### Synthesis of 81

The 81h (150 mg, 0.278 mmol, 1 equiv) was purified by chiral separation with the following conditions (Column: CHIRALPAK IC, 2*25 cm, 5 um; Mobile Phase A: Hex (0.5% 2M NH₃-MeOH), Mobile Phase B: EtOH:DCM=: 1; Flow rate: 20 mL/min; Gradient: 50% B to 50% B in 11 min; Wave Length: 220/254 nm; RT1 (min): 6.93; RT2 (min): 9.14; the first peak is product) to afford Compound 81 (26.2 mg, 17.34%) as a yellow solid.

LC-MS: (ES, m/z): [M+H]⁺ 539

H-NMR: 1H NMR (400 MHz, DMSO-d₆, ppm, δ): 0.78-0.95 (m, 4H), 1.44-1.49 (m, 1H), 1.58-1.67 (m, 4H), 1.71 (s, 3H), 1.87-1.89 (m, 1H), 2.76-2.81 (m, 3H), 2.95-3.00 (m, 1H), 3.25 (s, 1H), 3.49 (s, 3H), 4.53-4.69 (t, 1H), 4.61-4.69 (d, 2H), 7.01 (s, 1H), 7.25-7.27 (d, 1H), 7.31 (s, 1H), 7.42-7.46 (t, 1H), 7.66-7.71 (m, 2H), 7.78 (s, 1H), 8.34 (s, 1H).

### Example 82. Synthesis of Compound 82

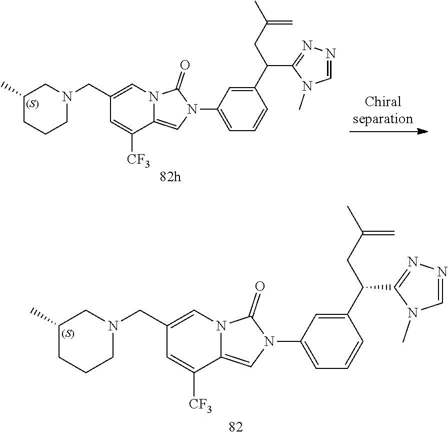

### Synthesis of 82

The Compound 82h (150 mg, 0,278 mmol, 1 equiv) was purified by chiral separation with the following conditions (Column: CHIRALPAK IC, 2*25 cm, 5 μm; Mobile Phase A: Hex (0.5% 2M NH₃-MeOH, Mobile Phase B: EtOH:DCM=1:1; Flow rate: 20 mL/min; Gradient: 50% B to 50% B in 11 min; Wave Length: 220/254 am; RT1 (min): 6.93; RT2 (min): 9.14; the second peak is product) to afford Compound 82 (36.1 mg, 23.99%) as a yellow solid.

LC-MS: (ES, m/z): [M+H]⁺ 539

H-NMR: 1H NMR (400 MHz, DMSO-d₆, ppm, δ): 0.78-0.95 (m, 4H), 1.44-1.49 (m, 1H), 1.58-1.67 (m, 4H), 1.71 (s, 3H), 1.87-1.89 (m, 1H), 2.76-2.81 (m, 3H), 2.95-3.00 (m, 1H), 3.25 (s, 1H), 3.49 (s, 3H), 4.53-4.69 (t, 1H), 4.61-4.69 (d, 2H), 7.01 (s, 1H), 7.25-7.27 (d, 1H), 7.31 (s, 1H), 7.42-7.46 (t, 1H), 7.66-7.71 (m, 2H), 7.78 (s, 1H), 8.34 (s, 1H).

### Example 83. Synthesis of Compound 83

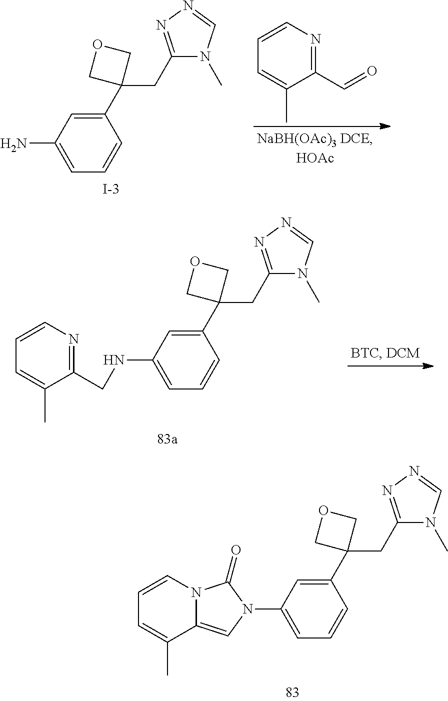

### Synthesis of 83a

To a stirred solution of I-3 (300 mg, 1.228 mmol, 1 equiv) and 3-methylpyridine-2-carbaldehyde (178.51 mg, 1,474 mmol, 1.2 equiv) in DCE was added HOAc (73.74 mg, 1.228 mmol, 1 equiv) and NaBH(OAc)₃ (520.53 mg, 2.456 mmol, 2 equiv). The resulting mixture was stirred overnight at room temperature. The reaction was quenched by the addition of water (10 mL). The resulting mixture was extracted with CH₂Cl₂ (3×20 mL). The combined organic layers were concentrated under reduced pressure. The residue was purified by Prep-TLC (CH₂Cl₂/MeOH 20:1) to afford 83a (160 mg, 37.29%) as a white solid.

### Synthesis of 83

To a stirred solution of 83a (150.00 mg, 0.429 mmol, 1.00 equiv) in DCM (10.00 mL) was added Triphosgene (44.58 mg, 0.150 mmol, 0.35 equiv) at 0 degrees C. under nitrogen atmosphere. The resulting mixture was stirred for 30 min at room temperature. The resulting mixture was washed with 10 mL of NaHCO₃ (aq.). The crude product was purified by Prep-HPLC with the following conditions (Column: XBridge Prep ODD C18 Column, 30*150 mm, 5 μm; Mobile Phase A: Water (10 mmol/L NH₄HCO₃), Mobile Phase B: MeOH-HPLC; Flow rate: 60 mL/min; Gradient: 40% B to 50% B in 7 min, Wave Length: 220 nm; RT1 (min): 6.28) to afford Compound 83 (42.5 mg, 25.58%) as a yellow solid.

LC-MS: (ES, m/z): [M+H]⁺ 376

H-NMR: (400 MHz, CD₃OD, ppm): δ 2.22 (s, 3H), δ 2.95 (s, 3H), δ 3.73 (s, 2H), δ 5.11 (s, 4H), δ 6.24-6.27 (m, 1H), δ 6.39-6.41 (d, 1H), δ 6.87-6.88 (d, 1H), δ 7.01 (s, 1H), δ 7.31-7.48 (m, 2H), δ 7.67-7.69 (m, 1H), δ 8.22 (s, 1H).

### Example 84. Synthesis of Compound 84

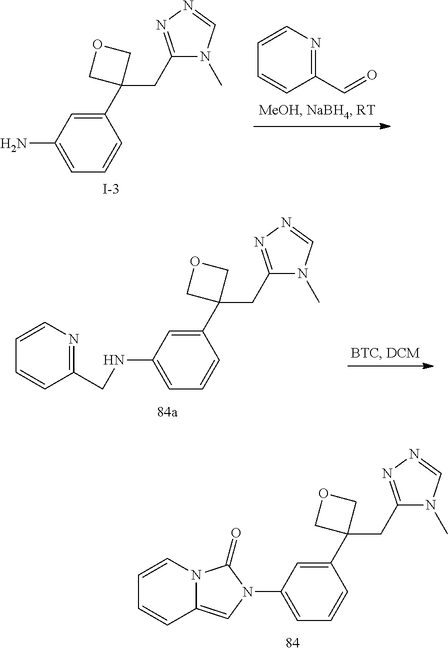

### Synthesis of 84a

To a stirred solution of 1-3 (200 mg, 0.819 mmol, 1 equiv) in MeOH (10 m) was added 2-formylpyridine (87.69 mg, 0.819 mmol, 1 equiv). The resulting mixture was stirred overnight at room temperature. To the above mixture was added NaBH₄ (30.97 mg, 0.819 mmol, 1 equiv). The resulting mixture was stirred for additional 2 h at room temperature. The reaction was quenched by the addition of NH₄Cl (aq.) (40 mL). The resulting mixture was extracted with EtOAc (2×40 mL). The combined organic layers were concentrated under reduced pressure. The residue was purified by Prep-TLC (CH₂Cl₂/MeOH 20:1) to afford 84a (200 mg, 72.84%) as a white solid.

### Synthesis of 84

To a stirred solution of 84a (220.00 mg, 0.656 mmol, 1.00 equiv) in DCM (10.00 mL) was added Triphosgene (64.23 mg 0.216 mmol, 0.33 equiv) at 0° C. The resulting mixture was stirred for 30 min at 0° C. The reaction was quenched by the addition of NaHCO₃ (aq.) (15 mL). The aqueous layer was extracted with CH₂Cl₂/MeOH=10/1 (2×30 mL). The resulting mixture was concentrated under vacuum. The crude product (300 mg) was purified by Prep-HPLC with the following conditions (Column: XBridge Prep OBD C18 Column, 30*150 mm, 5|Ìm; Mobile Phase A: Water (10 mmol/L NH₄HCO₃), Mobile Phase B: ACN; Flow rate: 60 mL/min; Gradient: 12% B to 20% B in 16 min, Wave Length: 220 nm; RT1 (min): 15.20) to afford Compound 84 (45.3 mg, 19.11%) as a yellow solid.

LC-MS: (ES, m/z): [M+H]⁺ 376

H-NMR: (400 MHz, CD₃OD, ppm): δ 2.95 (s, 3H), δ 3.67 (s, 2H), δ 5.11 (s, 4H), δ 6.25-6.27 (m, 1H), δ 6.56-6.58 (d, 1H), δ 6.87-6.89 (d, 1H), δ 7.01 (s, 1H), 67.03-7.05 (d, 1H), δ 7.29 (s, 1H), δ 7.41-7.44 (m, 1H), δ 7.54-7.56 (d, 1H), δ 7.63-7.65 (d, 1H), δ 8.22 (s, 1H).

### Example 85. Synthesis of Compound 85

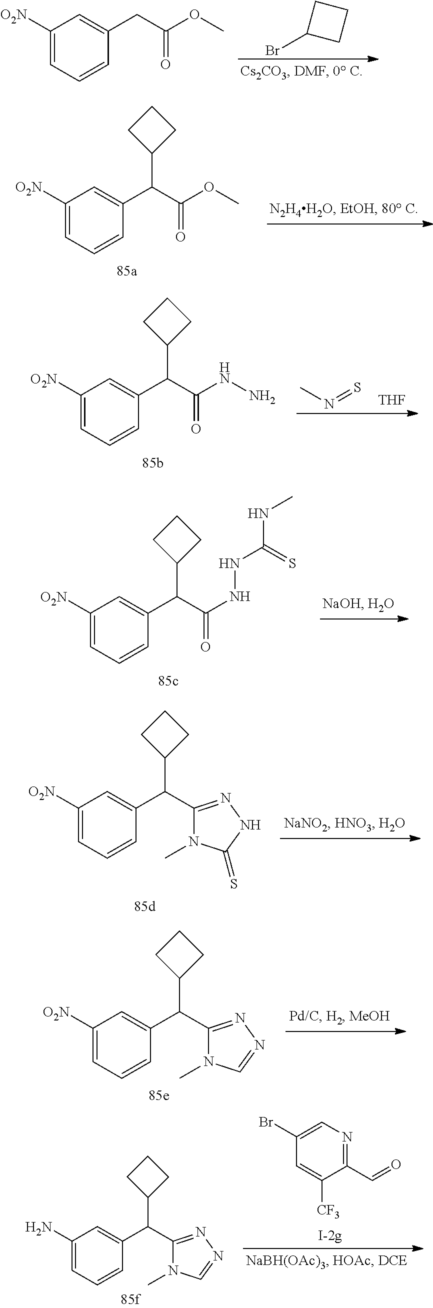

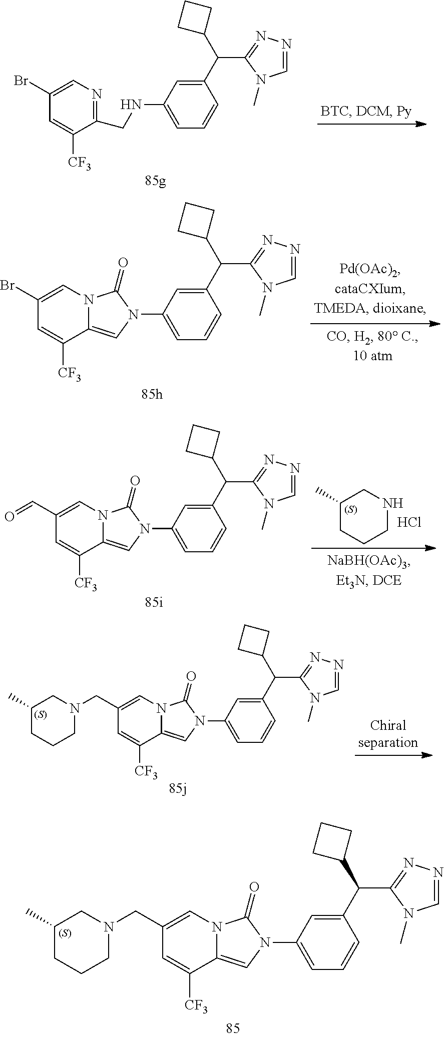

### Synthesis of 85a

A mixture of methyl 2-(3-nitrophenyl)acetate (48.1 g, 246.447 mmol, 1 equiv) and Cs₂CO₃ (401.49 g, 1232.235 mmol, 5 equiv) in DMF (500 mL) was stirred for 3h at 0° C. under nitrogen atmosphere. To the above mixture was added bromocyclobutane (99.81 g, 739.341 mmol, 3 equiv) at room temperature. The resulting mixture was stirred overnight at room temperature. The reaction was diluted with NH₄Cl (aq.) (3 L) at 0° C. The aqueous layer was extracted with EtOAc (3×500 mL). The resulting mixture was concentrated under reduced pressure. The residue was purified by silica gel column chromatography, eluted with PE/EA (150:1) to afford 85a (49 g, 73.38%) as a off-white solid

### Synthesis of 85b

To a stirred solution of 85a (49 g, 196.577 mmol, 1 equiv) in EtOH (500 mL) was added hydrazine hydrate (98%) (251.04 g, 4914.425 mmol, 25 equiv, 98%) at room temperature. The resulting mixture was stirred overnight at 80° C. The reaction was diluted by the addition of water (500 mL) at room temperature. The aqueous layer was extracted with CH₂Cl₂/MEOH (10:1) (3×500 mL). The resulting mixture was concentrated under reduced pressure. The residue was purified by silica gel column chromatography, eluted with CH₂Cl₂/MeOH (100:1) to afford 85b (43 g, 79.86%) as a yellow oil.

### Synthesis of 85c

To a stirred solution of 85b (45 g, 180.527 mmol, 1 equiv) in THE (450 mL) was added methyl isothiocyanate (33.00 g, 451.317 mmol, 2.5 equiv) at room temperature under nitrogen atmosphere. The resulting mixture was stirred for 3h at room temperature. The resulting mixture was diluted with water (280 mL). The resulting mixture was filtered, the filter cake was washed with water (3×50 mL). The resulting solid was dried under vacuum. This resulted in 85c (55 g, 86.00%) as a white solid.

### Synthesis of 85d

To a stirred solution of NaOH (66 g, 1650.120 mmol, 9.67 equiv) in H₂O (1.65 L) was added 85c (55 g, 170.606 mmol, 1 equiv) at room temperature. The resulting mixture was stirred overnight at room temperature. The mixture was acidified to pH 5 with HCl (1 M). The resulting mixture was filtered, the filter cake was washed with water (3×50 mL). The resulting solid was dried under vacuum. This resulted in 85d (50 g, 86.66%) as a off-white solid.

### Synthesis of 85e

To a stirred mixture of 85d (50 g, 164.274 mmol, 1 equiv) in EtOAc (190 mL) and H₂O (760 mL) was added NaNO₂ (113.3 g 1642.74 mmol, 10 equiv) at room temperature. To the above mixture was added HNO₃ (1642 mL, 1642.74 mmol, 10.00 equiv, 1 M) dropwise at 0 degrees C. The resulting mixture was stirred overnight at room temperature. The mixture was neutralized to pH 7 with saturated NaHCO₃ (aq.). The aqueous layer was extracted with CH₂Cl₂/MeOH (10:1) (3×500 mL). The resulting mixture was concentrated under reduced pressure. The residue was purified by silica gel column chromatography, eluted with CH₂Cl₂/MeOH (50:1) to afford 85e (40 g, 85.84%) as a yellow solid.

### Synthesis of 85f

To a solution of 85e (40 g, 146.892 mmol, 1 equiv) in 1.2 L MeOH was added Pd/C (20%, 8g) in a 2 L round-bottom flask. The mixture was hydrogenated at room temperature overnight under hydrogen atmosphere using a hydrogen balloon, filtered through a Celite pad, and concentrated under reduced pressure. This resulted in 85f (35 g, 94.39%) as a off white solid.

### Synthesis of 85g

To a stirred solution of 85f (31.45 g, 123.800 mmol, 1.2 equiv) and I-2g (31.45 g, 123.800 mmol, 1.2 equiv) in DCE (300 mL) were added NaBH(OAc)3 (43.73 g, 206.334 mmol, 2 equiv) and 1-OAc (6.20 g, 103,167 mmol, 1 equiv) at room temperature under nitrogen atmosphere. The resulting mixture was stirred overnight at room temperature under nitrogen atmosphere. The reaction was quenched by the addition of water (500 mL) at room temperature. The aqueous layer was extracted with EtOAc (3×500 mL), dried over anhydrous Na₂SO₄. After filtration, the filtrate was concentrated under reduced pressure. The residue was purified by trituration with MTBE (2×50 mL). This resulted in 85g (34 g, 65.18%) as a white solid,

### Synthesis of 85h

To a stirred solution of 85g (34 g, 70.784 mmol, 1 equiv) and pyridine (33.59 g, 424.704 mmol, 6 equiv) in DCM (400 mL) were added Triphosgene (7.35 g, 24,774 mmol, 0.35 equiv) dropwise at 0′C under nitrogen atmosphere. The resulting mixture was stirred for 10 min at room temperature under nitrogen atmosphere. The reaction was quenched by the addition of water (500 mL) at room temperature. The resulting mixture was extracted with CH₂Cl₂ (3×500 mL) and dried over anhydrous CaCl₂. After filtration, the filtrate was concentrated under reduced pressure. The residue was purified by trituration with MTBE (2×100 mL). This 85h (33 g, 87.47%) as a yellow solid.

### Synthesis of 85i

To a solution of 85h (33 g, 65.175 mmol, 1 equiv) and TMEDA (15.15 g, 130.350 mmol, 2 equiv) in dioxane (1000 mL) was added bis(adamantan-1-yl)(butyl)phosphane (4.67 g, 13.035 mmol, 0.2 equiv) and Pd(OAc)2 (1.46 g, 6.518 mmol, 0.1 equiv) in an autoclave. After flushing the autoclave three times with CO/H₂ (1:1), the mixture was pressurized to 10 atm with CO/H₂ (1:1) and run overnight at 80 degrees C. The resulting mixture was concentrated under vacuum. The residue was purified by silica gel column chromatography, eluted with CH₂Cl₂/MeOH (20:1) to CH₂Cl₂/MeOH (5:1) to afford 85i (20 g, 67.38%) as a yellow solid.

### Synthesis of 85j

To a stirred mixture of 85i (10 g, 21,957 mmol, 1 equiv) and (3S)-3-methylpiperidine hydrochloride (8.93 g, 65.871 mmol, 3 equiv) in DCE (150 mL) was added Et₃N (8.89 g, 87.828 mmol, 4 equiv). The resulting mixture was stirred for 2 h at room temperature under nitrogen atmosphere. To the above mixture was added NaBH(OAc)3 (6.98 g, 32.936 mmol, 1.5 equiv). The resulting mixture was stirred overnight at room temperature. The reaction was quenched by the addition of water (200 mL) at room temperature. The resulting mixture was extracted with CH₂Cl₂/MeOH=10/1 (2×200 mL). The combined organic layers were concentrated under reduced pressure. The residue was purified by reverse flash chromatography with the following conditions: column, C18 silica gel; mobile phase, MeCN in water (10 mol/L NH₄HCO₃), 15% to 60% gradient in 40 min; detector, UV 254 nm. This resulted in 85j (6.5 g, 54.96%) as a yellow solid.

### Synthesis of 85

The 85j (6.5 g) was purified by Prep-Chiral-SFC with the following conditions (Column: CHIRAL ART Cellulose-SB, 5*25 cm, 10 μm; Mobile Phase A: CO₂, Mobile Phase B: MEOH (0.1% 2M NH₃-MeOH); Flow rate: 200 mL/min; Gradient: isocratic 30% B; Column Temperature (° C.): 35; Back Pressure (bar): 100; Wave Length: 220 nm; RT2 (min): 6.26; Sample Solvent: MEOH (0.1% 2M NH₃-MEOH); Injection Volume: 1 mL; Number Of Runs: 30). This resulted in Compound 85 (3.0062g) as a yellow solid.

LC-MS: (ES, m/z): [M+H]⁺ 540

H-NMR: (400 MHz, DMSO-d6, ppm): δ 0.84-0.91 (m, 4H), δ 1.38-1.95 (m, 12H), δ 2.08-2.10 (m, 1H), δ 2.68-2.77 (m, 2H), δ 3.19-3.25 (m, 3H), δ 3.43 (s, 3H), δ 4.25-4.28 (d, 1H), δ 7.01 (s, 1H), δ 7.19-7.21 (d 1H), δ 7.32 (s, 1H), δ 7.43-7.46 (t, 1H), δ 7.66-7.75 (m, 3H), δ 8.34 (s, 1H).

### Example 86. Synthesis of Compound 86

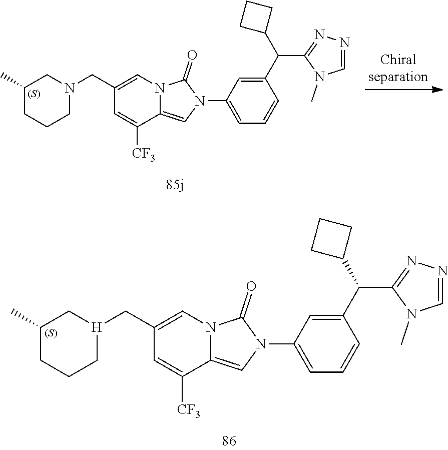

### Synthesis of 86

85j (85.00 mg, 0.158 mmol, 1.00 equiv) was purified by Prep-Chiral-HPLC with the following conditions (Column: CHIRAL ART Cellulose-SB, 2*25 cm, 5 μm; Mobile Phase A: Hex (0.5% 2M NH₃-MeOH), Mobile Phase B: EtOH; Flow rate: 20 mL/min; Gradient: 30% B to 30% B in 8.5 min; Wave Length: 220/254 nm; RT1 (min): 5.19) to afford Compound 86 (24.1 mg, 27.90%) as a yellow solid.

LC-MS: (ES, m/z): [M+H]⁺ 539

H-NMR: (400 MHz, CDCl₃, ppm): δ 0.81-0.83 (d, 4H), δ 1.66-1.78 (m, 1H), δ 2.04-2.11 (m, 1H), δ 3.65-3.69 (m, 1H), δ 2.72-3.73 (m, 1H), δ 3.22-3.35 (m, 3H), δ 3.42 (s, 3H), δ 4.24-4.28 (d, 1H), δ 7.00 (s, 1H), δ 7.19-7.22 (d, 1H), δ 7.33 (s, 1H), δ 7.44-7.47 (m, 1H), δ 7.70-7.72 (m, 3H), δ 8.32 (s, 1H).

### Example 87. Synthesis of Compound 87

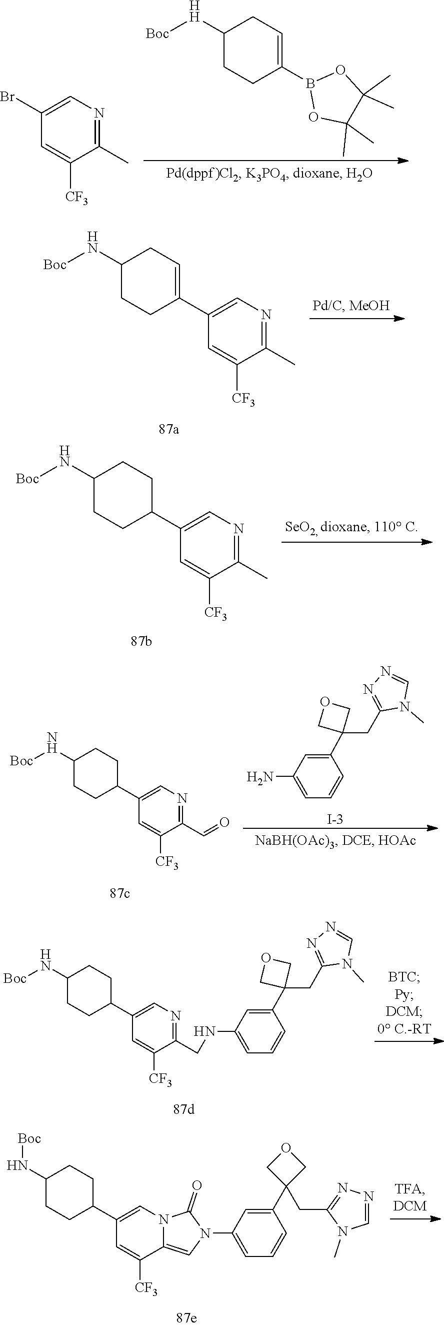

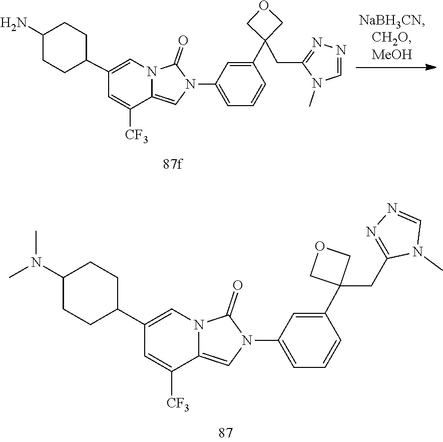

### Synthesis of 87a

Into a 20 mL sealed tube were added 5-bromo-2-methyl-3-(trifluoromethyl)pyridine (1.00 g, 4.166 mmol, 1.00 equiv), dioxane (8.00 mL), H₂O (2.00 mL), tert-butyl N-[4-(4,4,5,5-tetramethyl-1,3,2-dioxaborolan-2-yl)cyclohex-3-en-1-yl]carbamate (1.35 g, 4.176 mmol, 1.00 equiv), Pd(dppf)Cl₂ (0.30 g, 0.417 mmol 0.1 equiv), and K₃PO₄ (1.77 g, 8.333 mmol, 2 equiv) under nitrogen atmosphere. The resulting mixture was stirred for 6h at 80 degrees C. under nitrogen atmosphere. The resulting mixture was diluted with water (30 mL). The aqueous layer was extracted with EtOAc (3×20 mL). The resulting mixture was concentrated under vacuum. The residue was purified by Prep-TLC (PE/EtOAc 3:1) to afford 87a (1.1 g, 71.12%) as a colorless oil.

### Synthesis of 87b

Into a 50 mL round-bottom flask were added 87a (1.10 g, 3.087 mmol, 1 equiv), MeOH (30.00 mL), Pd/C (200.00 mg) under nitrogen atmosphere. The resulting mixture was stirred for 3h at room temperature under hydrogen atmosphere using a hydrogen balloon, filtered through a Celite pad and concentrated under reduced pressure. This resulted in 87b (920 mg, 79.84%) as a colorless oil.

### Synthesis of 87c

To a stirred solution of 87b (910 mg, 2,539 mmol, 1 equiv) in dioxane (20 mL) was added SeO₂ (845.19 mg, 7,617 mmol, 3 equiv). The resulting mixture was stirred overnight at 110° C. The resulting mixture was diluted with water (30 mL). The resulting mixture was extracted with EtOAc (2×20 mL). The combined organic layers were washed with brine (10 ml), dried over anhydrous Na₂SO₄. After filtration, the filtrate was concentrated under reduced pressure. This resulted in 87c (710 mg, 75.09%) as a yellow oil.

### Synthesis of 87d

To a stirred mixture of 87c (370 mg, 0.994 mmol, 1 equiv) and I-3 (242.73 mg, 0.994 mmol, 1 equiv) in DCE (15 mL) was added HOAc (59.67 mg, 0.994 mmol, 1 equiv) and NaBH(OAc)3 (421.16 mg, 1.988 mmol, 2 equiv) at room temperature. The resulting mixture was stirred overnight at room temperature. The reaction was quenched by the addition of water (10 mL). The resulting mixture was extracted with CH₂Cl₂/MeOH=10/1 (3×20 mL). The combined organic layers were concentrated under reduced pressure. The residue was purified by Prep-TLC (CH₂Cl₂/MeOH 10:1) to afford 87d (320 mg, 53.62%) as a light yellow solid.

### Synthesis of 87e

To a stirred solution of 87d (320 mg, 0.533 mml, 1 equiv) and Pyridine (252.83 mg, 3.198 mmol, 6 equiv) in DCM (10 mL) was added Triphosgene (63.23 mg, 0.213 mmol, 0.4 equiv). The resulting mixture was stirred for 2 h at 0° C. The reaction was quenched by the addition of NaHCO₃ (aq.) (15 mL). The resulting mixture was extracted With CH₂Cl₂/MeOH (10/1) (4×20 mL). The combined organic layers were concentrated under reduced pressure. The residue was purified by Prep-TLC (C12Cl₂/MeOH 10:1) to afford 87e (240 mg, 71.89%) as a yellow solid.

### Synthesis of 87f

To a stirred solution of 87e (240 mg, 0.383 mmol, 1 equiv) in DCM (3 mL) was added TFA (0.6 mL). The resulting mixture was stirred for 4 h at room temperature. The resulting mixture was concentrated under reduced pressure. The residue was purified by Prep-TLC (CH₂Cl₂/MeOH 8:1) to afford 87f (180 mg, 89.26%) as a yellow solid,

### Synthesis of 87

To a stirred solution of 87f (180 mg, 0.342 mmol, 1 equiv) and formaldehyde solution (83.22 ag, 1.026 mmol, 3 equiv, 37%) in MeOH (3 mL) was added HOAc (20.5 ag, 0.342 mmol, 1 equiv) and NaBH₃CN (42.96 mg, 0.684 mmol, 2 equiv) at 0° C. The resulting mixture was stirred for 1 h at 0° C. The reaction was quenched by the addition of NaHCO₃ (aq.) (15 mL). The resulting mixture was extracted with CH₂Cl₂/MeOH=10/1 (4×20 mL). The combined organic layers were concentrated under reduced pressure. The residue was purified by Prep-TLC (CH₂Cl₂₂/MeOH 8:1) to afford Compound 87 (49.1 mg, 25.90%) as a yellow solid.

LC-MS: (ES, m/z): [M+H]⁺ 556

H-NMR: (400 MHz, DMSO-d6, ppm): δ 1.48-1.58 (m, 4H), δ 1.66-1.75 (m, 2H), δ 1.93-1.96 (m, 4H), δ 2.19-2.33 (m, 6H), δ 2.97 (s, 1H), δ 3.54 (s, 2H), δ 4.91-4.96 (m, 4H), δ6.89-6.91 (d, 1H), δ 7.04 (s, 1H), δ 7.39-7.45 (m, 3H), δ 7.75-7.77 (d, 1H), δ 8.21 (s, 1H).

### Example 88. Synthesis of Compound 88

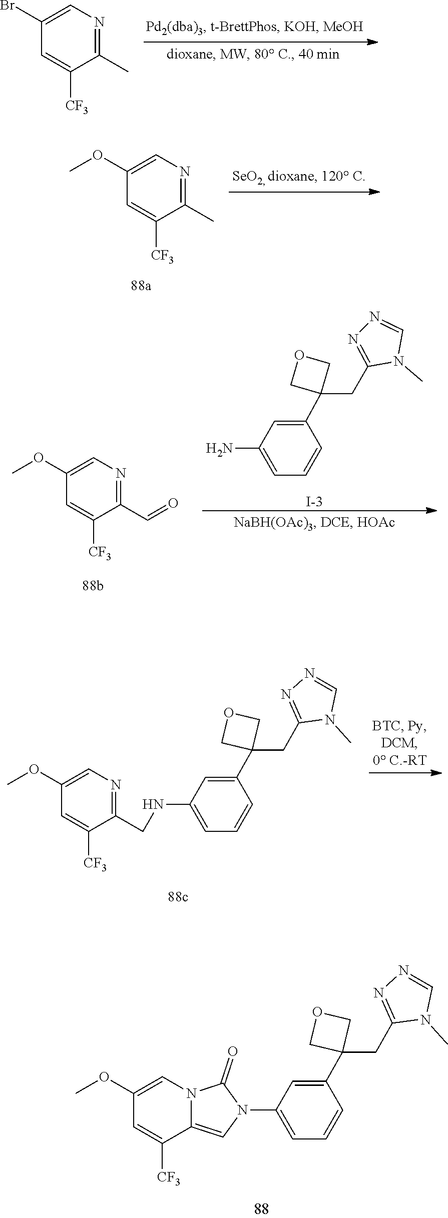

### Synthesis of 88a

To a stirred mixture of 5-bromo-2-methyl-3-(trifluoromethyl)pyridine. (1 g, 4.166 mmol, 1 equiv) and Pd₂(dba)₃ (0.38 g, 0,417 mmol, 0.1 equiv) bin MeOH (5 mL) were added dioxane (10 mL) and KOH (0.70 g, 12.498 mmol, 3 equiv) at room temperature under nitrogen atmosphere, To the above mixture was added t-Brettphos (0.40 g, 0,833 mmol, 0.2 equiv) in one portion at room temperature. The final reaction mixture was irradiated with microwave radiation for 40 min at 80° C. The reaction was quenched with NH₄Cl (aq.) at room temperature. The resulting mixture was extracted with EtOAc (3×50 mL). The combined organic, layers were washed with water (3×50 mL), dried over anhydrous Na₂SO₄. After filtration, the filtrate was concentrated under reduced pressure. The residue was purified by silica gel column chromatography, eluted with CH₂Cl₂/MeOH=40:1 to afford 88a (300 mg, 37.67%) as a colorless oil.

### Synthesis of 88b

A mixture of 88a (280 mg, 1.465 mmol, 1 equiv) and SeO₂ (487.60 mg, 4.395 mmol, 3 equiv) in 1,4-dioxane was stirred overnight at 110° C. under nitrogen atmosphere. The resulting mixture was washed with 3×20 mL of water. The resulting mixture was extracted with EtOAc (3×20 mL). The combined organic layers were washed with water (3×20 mL), dried over anhydrous Na₂SO₄. After filtration, the filtrate was concentrated under reduced pressure. The residue was purified by Prep-TLC (CH₂C7/MeOH=40:1) to afford 88b (60 mg, 19.97%) as a yellow oil.

### Synthesis of 88c

To a stirred mixture of 88b (580 mg, 2.827 mmol, 1 equiv) and 1-3 (1036.09 mg, 4.240 mmol, 1.5 equiv) in DCE were added HOAc (169.79 mg, 2.827 mmol, 1 equiv) and NaBH(OAc)3 (1797.72 mg, 8.481 mmol, 3 equiv) at room temperature under nitrogen atmosphere. The resulting mixture was stirred overnight at room temperature. The resulting mixture was diluted with water (20 mL). The aqueous layer was extracted with CH₂Cl₂ (2×10 mL). The resulting mixture was concentrated under reduced pressure. The residue was purified by Prep-TLC (CH₂Cl₂/MeOH=20:1) to afford 88c (700 mg, 51.41%) as a yellow solid.

### Synthesis of 88

To a stirred solution of 88c (90 mg, 0.208 mmol, 1 equiv) and pyridine (98.55 mg, 1.248 mmol, 6 equiv) in DCM (4 mL) was added triphosgene (21.56 mg, 0.073 mmol, 0.35 equiv) at 0° C. under nitrogen atmosphere. The resulting mixture was stirred for 5 min at 0° C. The resulting mixture was washed with 3×10 mL of water. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by Prep-TLC (CH₂C12/MeOH=10:1) to afford Compound 88 (42.1 mg, 42.01%) as a yellow solid.

LCMS: (ES, m/z): [M+H]⁺ 460.

H-NMR: (400 MHz, DMSO-d₆, ppm, δ): 2.82-3.01 (s, 3H), 3.53 (s, 2H), 3.77 (s, 3H), 4.91-5.02 (m, 4H), 6.88 (s, 1H), 6.94 (s, 1H), 7.24 (s, 1H), 7.33-7.40 (m, 2H), 7.69-7.77 (m, 1H), 8.20 (s, 1H).

### Example 89. Synthesis of Compound 89

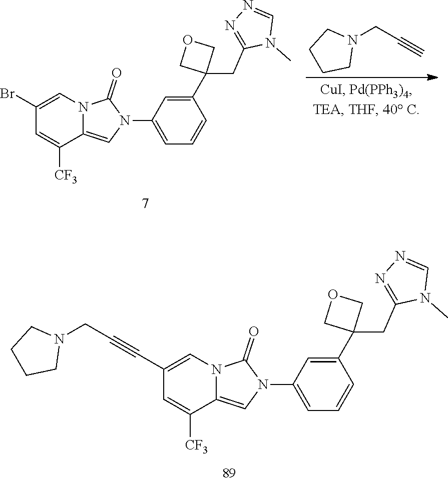

### Synthesis of 89

Into a 8 mL sealed tube were added 7 (150 mg, 0.295 mmol, 1.00 equiv), THF (2 mL), TEA (89.58 mg, 0.885 mmol, 3 equiv), 1-(prop-2-yn-1-ylpyrrolidine (48.33 mg, 0.443 mmol, 1.5 equiv), CuI (11.24 mg, 0.059 mmol, 0.2 equiv), and Pd(PPh₃)4 (34.10 mg, 0.029 mmol, 0.1 equiv) at room temperature. The resulting mixture was stirred for 12 h at 40° C. under nitrogen atmosphere. The reaction was quenched by the addition of water (10 mL) at room temperature. The aqueous layer was extracted with EtOAc (3×10 mL). The resulting mixture was concentrated under reduced pressure. The residue was purified by Prep-TLC (CH₂Cl₂/MeOH 10:1) to afford the crude product (55 mg). The crude product was purified by reverse phase flash with the following conditions (Column: Xselect CSH C18 OBD Column; Mobile Phase A: Water (0.1% F A), Mobile Phase B: ACN; Flow rate: 50 ml/min; Gradient: 44% B to 54% B in 7 min, Wave Length: 254) to afford Compound 89 (57.6 mg, 35.43%) as a yellow solid.

LC-MS: (ES, m/z): [M+H]⁺ 537

H-NMR: 1H NMR (400 MHz, DMSO-d6) δ 1.72-1.76 (m, 4H), δ 2.58 (s, 4H), 32.97 (s, 3H), δ 3.37 (s, 2H), 33.47-3.67 (m, 2H), δ 4.84-4.96 (m, 4H), δ 6.88-6.90 (d, 1H), δ 6.84 (s, 1H), δ 7.38-7.43 (m, 3H), δ 7.73-7.75 (t, 1H), δ 7.89 (s, 1H), δ 8.14 (s, 1H), δ 8.17-8.22 (t, 1H).

### Example 90. Synthesis of Compound 90

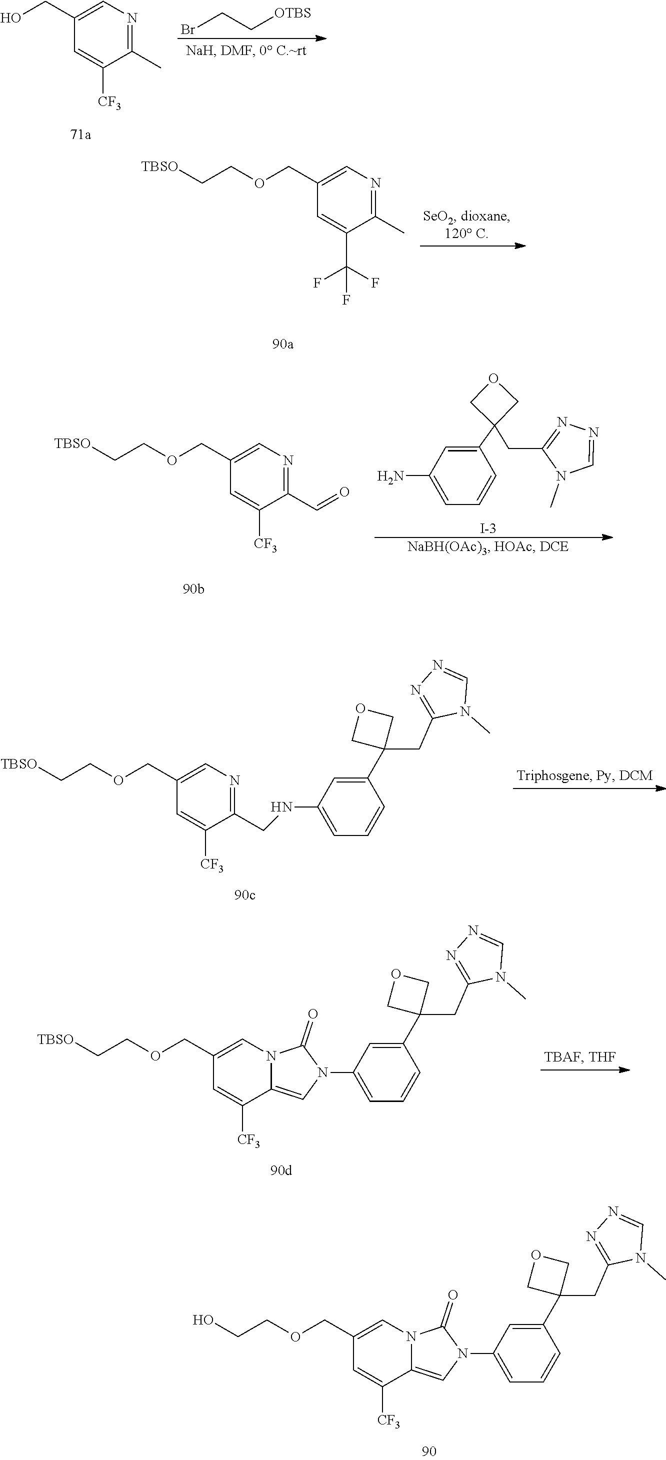

### Synthesis of 90a

To a stirred solution of 71a (1 g, 5.231 mmol, 1 equiv) in DMF (10 mL) were added NaH (0.25 g, 10.462 mmol, 2 equiv) in two portions at 0° C. The resulting mixture was stirred for 30 min at 0° C. under nitrogen atmosphere. To the above mixture was added (2-bromoethoxy)(tert-butyl)dimethylsilane (1.88 g, 7.846 mmol, 1.5 equiv) at 0° C. The resulting mixture was stirred for 1 h at room temperature under nitrogen atmosphere. The reaction was quenched by the addition of sat NH₄Cl (aq.) (50 mL) at room temperature. The aqueous layer was extracted with EtOAc (3×40 mL). The resulting mixture was concentrated under reduced pressure. The residue was purified by Prep-TLC (PE/EA=3:1) to afford 90a (220 mg, 12.03%) as colorless oil.

### Synthesis of 90b

To a stirred solution of 90a (300 mg, 0.858 mmol, 1 equiv) in dioxane (3 mL) were added SeO₂ (190.51 mg, 1.716 mmol, 2 equiv) at 0° C. under nitrogen atmosphere. The resulting mixture was stirred for 4 h at 120° C. The resulting mixture was diluted with water (50 mL). The aqueous layer was extracted with EtOAc (2×10 mL). The resulting mixture was concentrated under reduced pressure. The residue was purified by Prep-TLC (PE/EA=1:1) to afford 90b (140 mg, 40.38%) as colorless oil.

### Synthesis of 90c

To a stirred solution of 90b (260 mg, 0,715 mmol, 1 equiv) and 1-3 (192.24 mg, 0,786 mmol, 1.1 equiv) in DCE (3 mL) were added NaBH(OAc)3 (454.84 mg, 2.145 mmol, 3 equiv) and HOAc (42.96 mg, 0.715 mmol, 1 equiv) at room temperature. The resulting mixture was stirred for 6 h at room temperature. The resulting mixture was diluted with sat NH₄Cl (aq.) (60 ml). The aqueous layer was extracted with EtOAc (3×30 mL). The resulting mixture was concentrated under reduced pressure. The residue was purified by Prep-TLC (CH₂Cl₂/MeOH=40:1) to afford 90c (240 mg, 54.99%) as a yellow solid.

### Synthesis of 90d

To a stirred solution of 90c (220 mg, 0.372 mmol, 1 equiv) and Pyridine (176.45 mg, 2,232 mmol, 6 equiv) in DCM (8 mL) were added Triphosgene (38.61 mg, 0.130 mmol, 0.35 equiv) at 0° C. under nitrogen atmosphere. The resulting mixture was stirred for 5 min at 0° C. under nitrogen atmosphere. The reaction was quenched by the addition of sat NaHCO₃ (aq.) (30 mL) at room temperature. The aqueous layer was extracted with CH₂Cl₂ (3×20 mL). The resulting mixture was concentrated under reduced pressure. The residue was purified by Prep-TLC (CH₂Cl₂/MeOH=30:1) to afford 90d (170 mg, 74.02%) as a yellow solid.

### Synthesis of 90

A solution of 90d (160 mg, 0.259 mmol, 1.00 equiv) and TBAF (0.48 mL) in THF (2 mL) was stirred for 2h at room temperature under nitrogen atmosphere. The mixture was acidified to pH 6 with saturated NH₄Cl (aq.) (5 mL). The reaction was quenched with water (10 mL) at room temperature. The aqueous layer was extracted with EtOAc (3×10 mL). The resulting mixture was concentrated under reduced pressure. The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 12% B to 34% B in 9 min; Wave Length: 220 am; RT1 (min): 8.82) to afford Compound 90 (47.7 mg, 36.58%) as a yellow solid.

LC-MS: (ES, m/z): [M+H]⁺ 504

H-NMR: (400 MHz, DMSO, δ ppm): 2.97 (s, 3H), 3.45-3.56 (m, 6H), 4.32 (s, 2H), 4.67-4.69 (t, 1H), 4.91-4.96 (m, 4H), 6.88-6.90 (d, 1H), 7.06 (s, 1H), 7.73-7.74 (m, 3H), 7.75-7.78 (m, 2H), 8.21 (s, 1H).

### Example 91. Synthesis of Compound 91

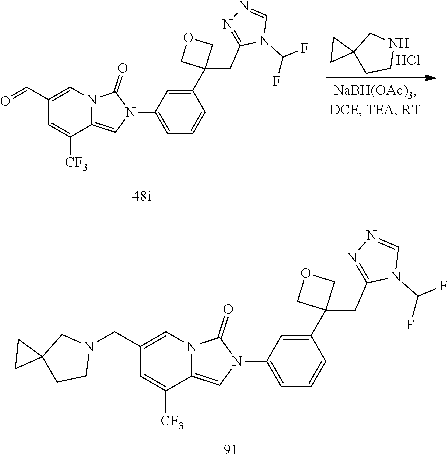

### Synthesis of 91

To a stirred mixture of 48i (200 mg, 0.405 mmol, 1 equiv) and 5-azaspiro[2.4]heptane hydrochloride (162.49 mg, 1.215 mmol, 3 equiv) in DCE (4 mL) was added TEA (164.07 mg, 1.620 mmol, 4 equiv) and NaBH(OAc)₃ (171.82 mg, 0.810 mmol, 2 equiv) at room temperature. The resulting mixture was stirred overnight at room temperature. The reaction was quenched by the addition of water (10 mL). The resulting mixture was extracted with CH₂Cl₂/MeOH=10/1 (2×10 mL). The combined organic layers were concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 55% B in 8 min, Wave Length: 220 nm; RT1 (min): 7.62) to afford Compound 91 (52.1 mg, 22.37%) as a yellow solid.

LC-MS: (ES, m/z): [M+H]⁺ 575

H-NMR: (400 MHz, DMSO-d6, ppm): δ 0.50-0.53 (d, 4H), δ 1.74-1.78 (m, 1H), δ 2.47-2.49 (m, 2H), δ 2.67-2.69 (m, 2H), δ 3.41 (s, 2H), δ 3.75 (s, 2H), δ 4.96 (s, 4H), δ 7.06-7.08 (m, 1H), δ 7.32 (s, 1H), δ 7.38-7.42 (m, 1H), δ 7.54-7.55 (m, 2H), δ 7.70-7.75 (m, 2H), δ 8.83 (s, 1H).

### Example 92. Synthesis of Compound 92

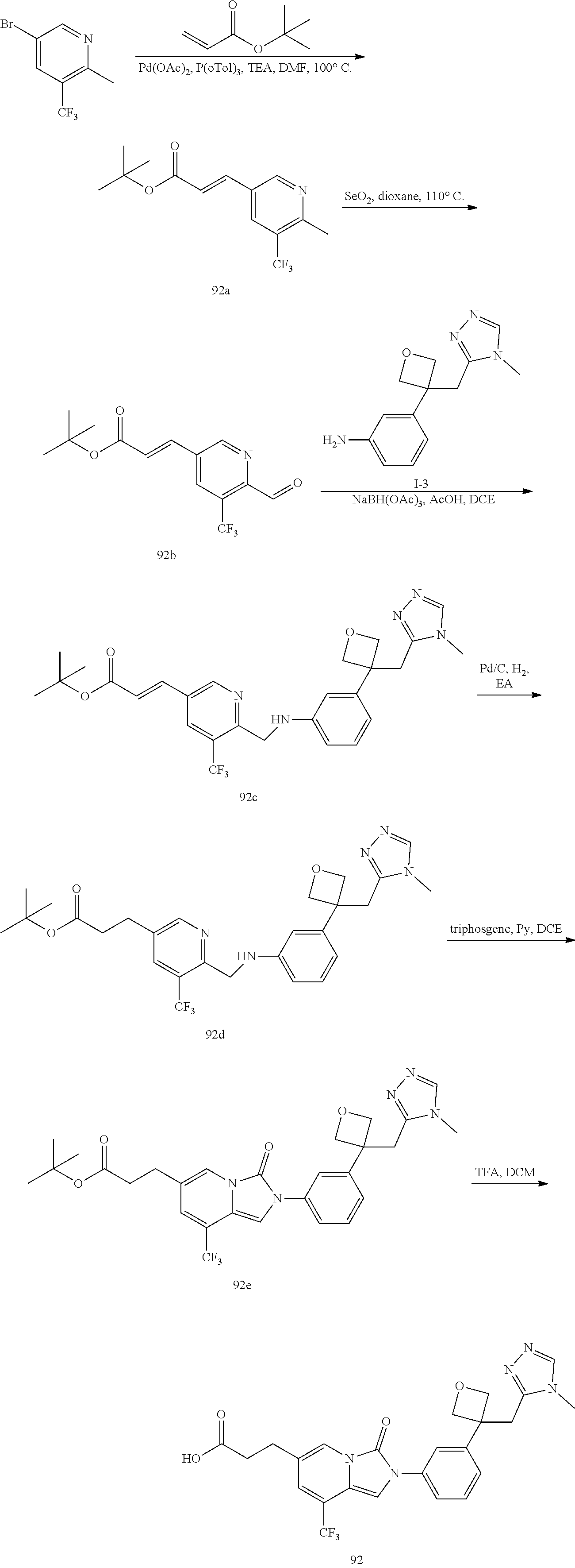

### Synthesis of 92a

To a stirred solution of 5-bromo-2-methyl-3-(trifluoromethyl)pyridine (1 g, 4.166 mmol, 1 equiv) and tert-butyl prop-2-enoate (0.53 g, 4.166 mmol, 1 equiv) in DMF (10 mL) were added Pd(OAc)2 (0.09 g, 0.417 mmol, 0.1 equiv), TEA (1.26 g, 12.498 mmol, 3 equiv) and P(o-Tol)₃ (0.51 g, 1.666 mmol, 0.4 equiv) at room temperature under nitrogen atmosphere. The resulting mixture was stirred overnight at 100° C. under nitrogen atmosphere. The reaction was quenched with Water/Ice (30 mL) at room temperature. The aqueous layer was extracted with EtOAc (3×15 mL). The resulting mixture was concentrated under reduced pressure. The residue was purified by Prep-TLC (PE/EA 6:1) to afford 92a (450 mg, 37.60%) as colorless oil.

### Synthesis of 92b

A solution of 92a (420 mg, 10462 mmol, 1 equiv) and SeO₂ (420.15 mg, 3.787 mmol, 2.59 equiv) in dioxane (4 mL) was stirred overnight at 110° C. under nitrogen atmosphere. The resulting mixture was filtered; the filter cake was washed with CH₂Cl₂ (3×6 mL). The filtrate was concentrated under reduced pressure. This resulted in 92b (300 mg, 54.49%) as brown oil.

### Synthesis of 92c

Into a 100 mL round-bottom flask were added 92b (280 g, 0.929 mmol, 1 equiv), DCE (4 mL) and I-3 (272.46 mg, 1.115 mol, 1.2 equiv) at room temperature. The resulting mixture was stirred for 30 min at room temperature. To the above mixture was added AcOH (55.81 mg, 0.929 mmol, 1 equiv) and NaBH(OAc)₃ (393.96 mg, 1.858 mmol, 2 equiv) at room temperature. The resulting mixture was stirred overnight at room temperature. The reaction was quenched with sat. NH₄Cl (aq.) (50 mL) at room temperature. The aqueous layer was extracted with EtOAc (3×10 mL). The resulting mixture was concentrated under reduced pressure. The residue was purified by Prep-TLC (CH₂Cl₂/MeOH 20:1) to afford 92c (300 mg, 60.95%) as colorless oil.

### Synthesis of 92d

To a solution of 92c (300 mg, 0.567 mmol, 1 equiv) in EA (8 ml) was added Pd/C (74.76 mg) in a pressure tank. The mixture was hydrogenated at room temperature overnight under hydrogen atmosphere using a hydrogen balloon, filtered through a Celite pad, and concentrated under reduced pressure. This resulted in 92d (280 mg, 92.98%) as colorless oil.

### Synthesis of 92e

To a stirred mixture 92d (280 mg, 0.527 mmol, 1.00 equiv) and Pyridine (249.99 mg, 3.162 mmol, 6 equiv) in DCE (5.00 mL) was added triphosgene (54.70 mg, 0.184 mmol, 0.35 equiv) at room temperature under nitrogen atmosphere. The resulting mixture was stirred for 30 min at room temperature under nitrogen atmosphere. The reaction was quenched with sat NaHCO₃ (aq.) (30 mL) at room temperature. The aqueous layer was extracted with EtOAc (3×7 mL). The resulting mixture was concentrated under reduced pressure. The residue was purified by Prep-TLC (CH₂Cl₂/MeOH 5:1) to afford 92e (200 mg, 68.10%) as a yellow solid

### Synthesis of 92

A solution of 92e (200 mg, 0.359 mmol, 1.00 equiv) in DCM (1.6 mL) and TFA (0.4 mL) was stirred overnight at room temperature. The resulting mixture was concentrated under reduced pressure. The residue was purified by Prep-TLC (CH Cₕ/MeOH 10:1) to afford crude product (150 mg). The crude product (150 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 5% B to 25% 13 in 12 min; Wave Length: 220 nm; RT1 (min): 11.33 to afford Compound 92 (30.2 mg, 16.79%) as a yellow solid.

LC-MS: (ES, m/z): [M+H]⁺ 502

H-NMR: (400 MHz, DMSO, δ ppm): 2.45-2.51 (m, 2H), 2.61-2.67 (m, 2H), 2.96 (s, 3H), 3.53 (s, 2H), 4.90-4.95 (m, 4H), 6.87-6.89 (d, 1H), 7.12 (s, 1H), 7.28 (s, 1H), 7.37-7.41 (t, 2H), 7.58 (s, 1H), 7.74 (s, 1H), 7.76 (s, 1H), 8.20 (s, 1H).

### Example 93. Synthesis of Compound 93

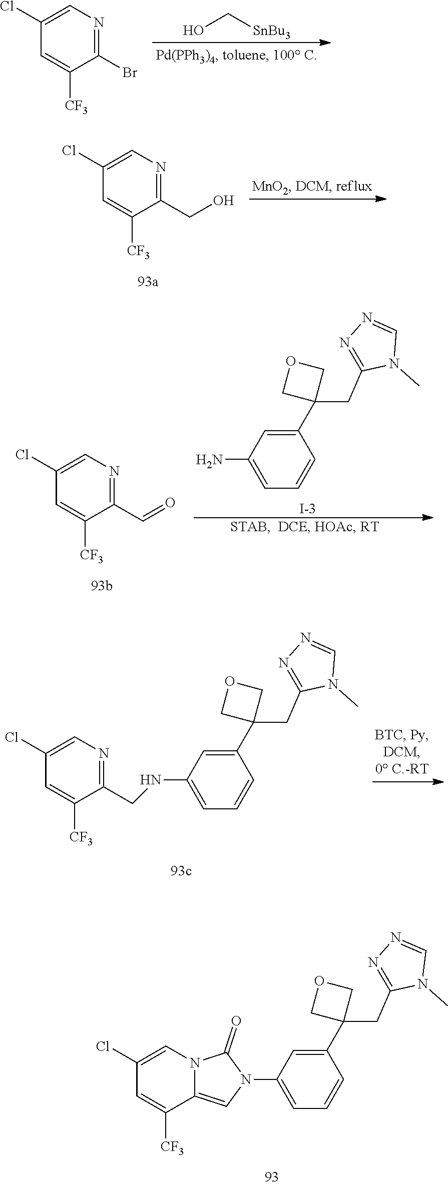

### Synthesis of 93a

To a stirred solution of 2-bromo-5-chloro-3-(trifluoromethyl) pyridine (1.6 g, 6.143 mmol, 1 equiv) and (tributylstannyl)methanol (2.96g, 9,214 mmol, 1.5 equiv) in Toluene (20 mL) were added Pd(PPh₃)₄ (0.71 g, 0,614 mmol, 0.1 equiv) at room temperature under nitrogen atmosphere. The resulting mixture was stirred overnight at 100° C. under nitrogen atmosphere. The reaction was quenched by the addition of water (30 mL) at room temperature. The resulting mixture was extracted with EtOAc (3×3 mL), dried over anhydrous Na₂SO₄. After filtration, the filtrate was concentrated under reduced pressure. The residue was purified by silica gel column chromatography elated with PE/EA (2:1) to afford 93a (600 mg, 41.55%) as a colorless oil.

### Synthesis of 93b

To a stirred solution of 93a (600 mg, 2.836 mmol, 1 equiv) in DCM (8 mL) were added MnO₂ (2465.48 mg, 28.360 mmol, 10 equiv) at room temperature under nitrogen atmosphere. The resulting mixture was stirred for 3h at 50° C. under nitrogen atmosphere. The resulting mixture was filtered by filter paper; the filter cake was washed with DCM (2×10 mL). The filtrate was concentrated under reduced pressure. The residue was purified by Prep-TLC (PE/EA 5:1) to afford 93b (260 mg, 40.25%) as a colorless oil.

### Synthesis of 93c

To a stirred solution of 93b (260 mg, 1.241 mmol, 1 equiv) and 1-3 (303.11 mg, 1.241 mmol, 1 equiv) in DCE (4 mL) were added STAB (525.93 mg, 2,482 mmol, 2 equiv) and AcOH (74.51 mg, 1.241 mmol, 1 equiv) at room temperature. The resulting mixture was stirred overnight at room temperature. The reaction was quenched by the addition of sat. NH₄Cl (aq.) (10 mL) at room temperature. The resulting mixture was extracted with EtOAc (3×10 mL) then dried over anhydrous Na₂SO. After filtration, the filtrate was concentrated under reduced pressure. The residue was purified by Prep-TLC (CH₂Cl₂/MeOH 20:1) to afford 93c (230 mg, 39.37%) as a yellow oil.

### Synthesis of 93

To a stirred solution of 93c (220 mg, 0.502 mmol, 1 equiv) and Pyridine (238.46 mg, 3012 mmol, 6 equiv) in DCE (3 mL) were added Triphosgene (52.19 mg, 0.176 mmol, 0.35 equiv) at 0° C. under nitrogen atmosphere. The resulting mixture was stirred for 10 min at room temperature under nitrogen atmosphere. The reaction was quenched by the addition of sat NaHCO₃ (aq.) (5 mL) at room temperature. The resulting mixture was extracted with CH₂Cl₂ (2×10 mL), dried over anhydrous CaCl₂. After filtration, the filtrate was concentrated under reduced pressure. The residue was purified by Prep-TLC (CH₂Cl₂/MeOH 15:1) to afford Compound 93 (145.7 mg, 61.64%) as a yellow solid,

LC-MS: (ES, m/z): [M+H]⁺:464

H-NMR: (400 MHz, DMSO-d6, ppm): δ 2.50-2.51 (m, 3H), δ 2.48-2.54 (m, 2H), δ 4.91-4.96 (m, 4H), δ 6.89-6.91 (d, 1H), δ 7.14 (s, 1H), δ 7.38-7.46 (m, 3H), δ 7.73-7.75 (m, 1H), δ 7.98 (m, 1H), δ 8.22 (s, 1H).

### Example 94. Synthesis of Compound 94

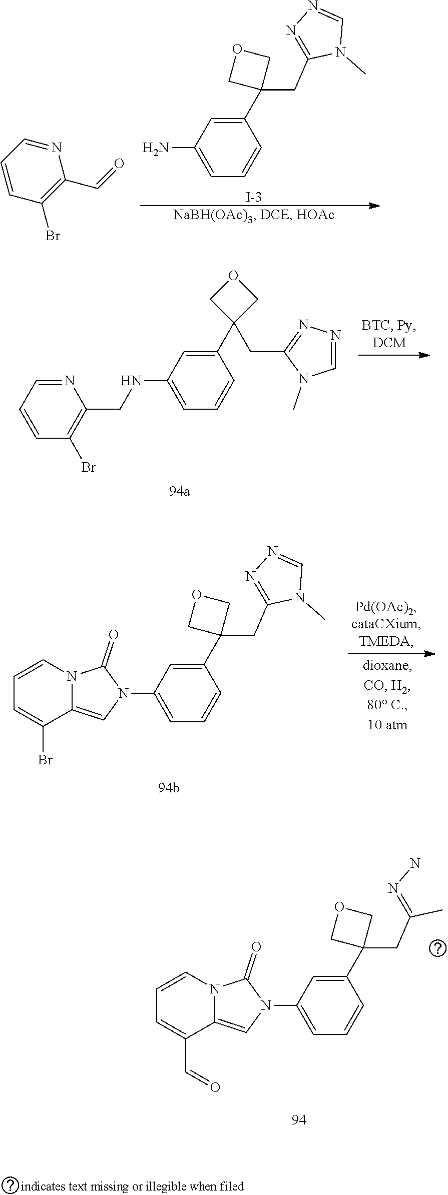

### Synthesis of 94a

To a stirred solution of 3-bromopyridine-2-carbaldehyde (800 mg, 4.30 mmol, 1.0 equiv) and 1-3 (1.0 g, 4.30 mmol, 1.0 equiv) in DCE (10 mL) were added NaBH(OAc)₃ (1.8 g, 8.60 mmol, 2.0 equiv) and HOAc (258 mg, 4.30 mmol, 1.0 equiv) at 0° C. The resulting mixture was stirred for 6h at 0° C. The reaction was quenched with sat. NH₄Cl (aq.) (60 ml) at room temperature. The aqueous layer was extracted with EtOAc (3×40 mL). The resulting mixture was concentrated under vacuum. The residue was purified by Prep-TLC (PE/EA=5:1) to afford 94a (800 mg, 42%) as a white solid.

### Synthesis of 94b

To a stirred solution of 94a (600 tug, 1.44 mmol, 1.0 equiv) and Pyridine (687 mg, 8.68 mmol, 6.0 equiv) in DCE (10 mL) were added triphosgene (150 mg, 0.50 mmol, 0.35 equiv) at 0° C. The resulting mixture was stirred for 0.5h at 0° C. The reaction was quenched with saturated NaHCO₃ (aq.) (50 mL) at room temperature. The aqueous layer was extracted with EtOAc (3×40 mL). The resulting mixture was concentrated under vacuum. The residue was purified by Prep-TLC (CH₂Cl₂/MeOH=10:1) to afford 94b (360 mg, 56%) as a white solid.

### Synthesis of 94

To a stirred solution of 94b (410 mg, 01931 mmol, 1 equiv) and CataCxium (333.36 mg, 0.931 mmol, 1 equiv) in dioxane (10 mL) were added TMEDA (324.62 mg 2.793 mmol, 3 equiv) and Pd(OAc)₂ (2090.59 mg, 9.310 mmol, 10 equiv) at room temperature under nitrogen atmosphere. The mixture was purged with nitrogen and then was pressurized to 10 atm with carbon monoxide/hydrogen (1:1) at 80° C. overnight. The resulting mixture was diluted with water (20 mL). The aqueous layer was extracted with EtOAc (2×10 mL). The residue was purified by Prep-TLC (CH₁₂Cl₂/MeOH=12:1) to afford crude product (100 mg). The crude product (100 mg) was purified by reverse flash chromatography with the following conditions (column, C18 silica gel; Mobile Phase A: Water (10 mmol/L NH₄HCO₃), Mobile Phase B: ACN; Flow rate: 40 mL/min; Gradient: 50% B to 60% B in 10 min; Wave Length: 254 nm to afford Compound 94 (50 mg, 15%) as a purple solid.

LC-MS: (ES, m/z): [M+H]⁺ 390

H-NMR: (400 MHz, dmso-d6, δ ppm): 2.96 (s, 3H), 3.54 (s, 2H), 4.79-5.00 (m, 4H), 6.42-6.46 (m, 1H), 6.85-6.87 (d, 1H), 7.31-7.48 (m, 2H), 7.51 (s, 1H), 7.54-7.56 (d, 2H), 7.72-7.77 (d, 1H), 7.93-7.95 (d, 1H), 8.21-8.23 (d, 1H), 9.82 (s, 1H).

### Example 95. Synthesis of Compound 95

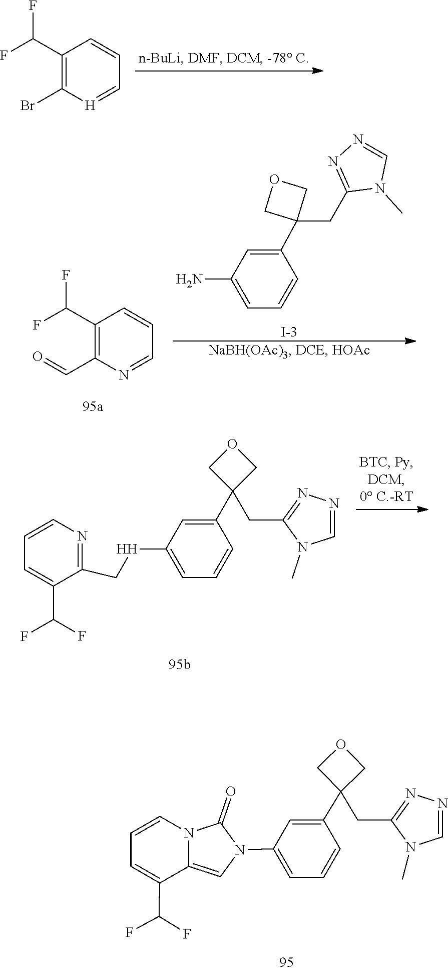

### Synthesis of 95a

In a 50-mL round bottom flask, to a solution of 2-bromo-3-(difluoromethyl) pyridine (1.0 g, 4.80 mmol, 1.0 equiv) in THF (10 mL) was added dropwise n-butyllithium solution (1.5 M in hexane, 2.5 mL, 1.5 mmol, 0.3 equiv) at −78 degrees C. under nitrogen atmosphere. The reaction mixture was stirred at −78 degrees C. for 30 min. Then a solution of DMF (40 mg, 0.48 mmol, 0.1 equiv) in 0.5 mL THF was added dropwise and the mixture was stirred for another 120 min. The reaction was quenched with sat. NH₄Cl (aq.) (30 mL), and then the mixture was extracted with EtOAc (3×:20 mL). The combined organic extracts were washed with brine (10 mL), dried over anhydrous Na₂SO₄, and filtered. The resulting mixture was concentrated under reduced pressure. The residue was purified by Prep-TLC (CH₂Cl₂/MeOH=50:1) to afford 95a (250 mg, 33%) as a yellow oil.

### Synthesis of 95b

To a stirred solution of 1-3 (100 mg, 0.63 mmol, 1.0 equiv) and 95a (233 mg, 0.95 mmol, 1.5 equiv) in DCE (3 ml) were added HOAc (38 mg, 0.63 mmol, 1.0 equiv) and NaBH(OAc)3 (404 mg, 1.90 mmol, 3.0 equiv) at room temperature. The resulting mixture was stirred overnight at room temperature. The reaction was quenched with sat. NH₄Cl (aq.) (10 ml) at room temperature. The resulting mixture was extracted with CH₂Cl₂ (3×10 mL). The resulting mixture was concentrated under reduced pressure. The residue was purified by Prep-TLC (CH₂Cl₂/MeOH=10:1) to afford 95b (80 mg, 29%) as a yellow solid.

### Synthesis of 95

To a stirred solution of 95b (80 mg, 0.20 mmol, 1.0 equiv) and Pyridine (98 mg, 1.24 mmol, 6.0 equiv) in DCE (2 ml) was added triphosgene (21 mg, 0.07 mmol, 0.35 equiv) at 0 degrees C. under nitrogen atmosphere. The resulting mixture was stirred for 20 min at room temperature under nitrogen atmosphere. The reaction was quenched with sat. NaHCO₃ (aq.) (10 ml) and extracted with CH₂Cl₂ (3×10 mL). The resulting mixture was concentrated under reduced pressure. The residue was purified by Prep-TLC (CH₂Cl₂/MeOH=15:1) to afford Compound 95 (31 mg, 36%) as a yellow solid.

LC-MS: (ES, m/z): [M+H]⁺ 412

H-NMR: (400 MHz, dmso-d6, δ ppm): 2.96 (s, 3H), 3.53 (s, 2H), 4.90-4.96 (t, 4H), 6.24-6.28 (m, 1H), 6.86-6.88 (m, 2H), 6.96-7.10 (m, 1H), 7.26 (s, 1H), 7.38-7.42 (m, 2H), 7.67-7.69 (d, 1H), 7.73-7.76 (m, 1H), 8.20 (s, 1H).

### Example 96. Synthesis of Compound 96

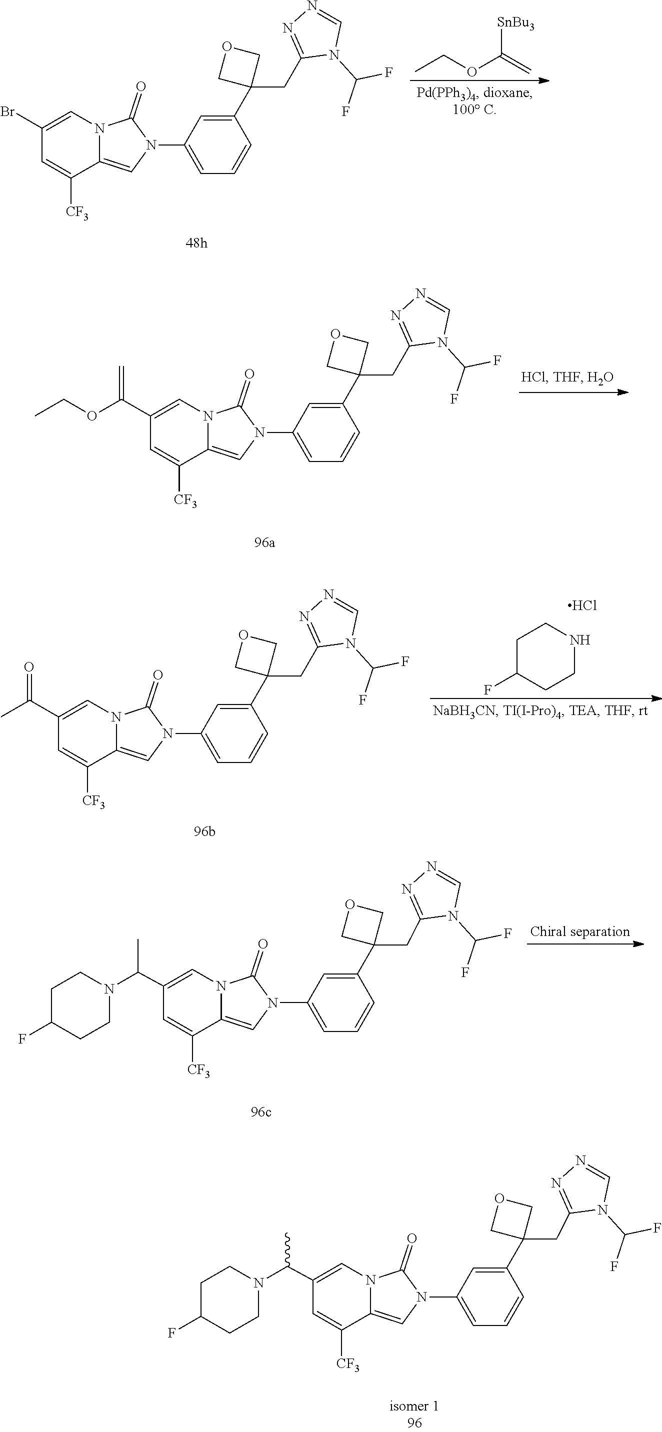

### Synthesis of 96a

To a stirred solution of 48h (2 g, 3,675 mmol, 1.00 equiv) and tributyl(1-ethoxyethenyl)stannane (1.73 g, 4,777 mmol, 1.3 equiv) in dioxane (20 mL) was added Pd(PPh₃)₄ (0.42 g, 0.363 mmol, 0.10 equiv) at room temperature under nitrogen atmosphere. The resulting mixture was stirred overnight at 100 degrees C. under nitrogen atmosphere. The resulting mixture was diluted with water (50 mL). The aqueous layer was extracted with EtOAc (2×50 mL). The residue was purified by silica gel column chromatography, eluted with CH₂Cl₂/MeOH (12:1) to 96a (1.5 g, 75.47%) as a yellow solid.

### Synthesis of 96b

To a stirred solution of 96a (1.5 g, 2,801 mmol, 1.00 equiv) in THF (10 mL) was added HCl (10 mL, 1M) at room temperature. The resulting mixture was stirred for 3h at room temperature. The reaction was quenched with NaHCO₃ (aq.) at room temperature. The resulting mixture was extracted with EtOAc (3×30 mL). The combined organic layers were dried over anhydrous Na₂SO₄. After filtration, the filtrate was concentrated under reduced pressure. The residue was purified by Prep-TLC (CH₂Cl₂/MeOH 12:1) to afford 96b (1.2 g, 82.73%) as a yellow solid.

### Synthesis of 96c

To a stirred solution of 96b (500 mg, 0,985 mmol, 1.00 equiv) and 4-fluoropiperidine hydrochloride (304.89 mg, 2.956 mmol, 3.00 equiv) in THF (10 mL) was added TEA (997.10 mg, 9.854 mmol, 10.00 equiv) and tetrakis(propan-2-yloxy)titanium (840.18 mg, 2.955 mmol, 3 equiv) at room temperature under nitrogen atmosphere. The resulting mixture was stirred for 3h at 50 degrees C. under nitrogen atmosphere. To the above mixture was added NaBH₃CN (185.77 mg, 2.955 mmol, 3 equiv) at room temperature. The resulting mixture was stirred overnight at room temperature. The reaction was quenched with saturated aqueous NH₄Cl at room temperature. The aqueous layer was extracted with CH₂Cl₂ (2×10 mL). The residue was purified by Prep-TLC (CH₂Cl₂/MeOH 15:1) to afford 96c (300 mg, 49.16%) as a yellow solid.

### Synthesis of 96

96c (300 mg) was purified by chiral separation with the following conditions (Column: CHIRALPAK IC, 2*25 cm, 5 μm; Mobile Phase A: Hex (0.5% 2M NH-MeOH), Mobile Phase B: EtOH:DCM=1:1; Flow rate: 20 mL/min; Gradient: 50% B to 50% B in 16.5 min; Wave Length: 220/254 nm; RT1 (min): 10.56, RT2 (min): 13.76, the first peak is product) to afford Compound 96 (106.1 mg, 34.31%) as a yellow solid.

LC-MS: (ES, m/z): [M+H]⁺ 595

H-NMR: (400 MHz, DMSO-d6, ppm): (400 MHz, DMSO-d6, ppm): δ 1.26-1.27 (d, 1H), δ 1.68-1.80 (m, 2H), δ 2.33-2.36 (m, 2H), δ 2.51-2.57 (m, 2H), δ 3.50-3.51 (m, 1H), δ 3.75 (s, 2H), δ 4.55-4.66 (m, 1H), δ 4.96 (s, 4H), δ 7.06-7.08 (d, 2H), δ 7.33 (s, 1H), δ 7.39-7.43 (m, 1H), δ 7.54-7.61 (m, 2H), δ 7.69 (s, 1H), δ 7.72-7.75 (m, 1H), δ 8.84 (s, 1H).

### Example 97. Synthesis of Compound 97

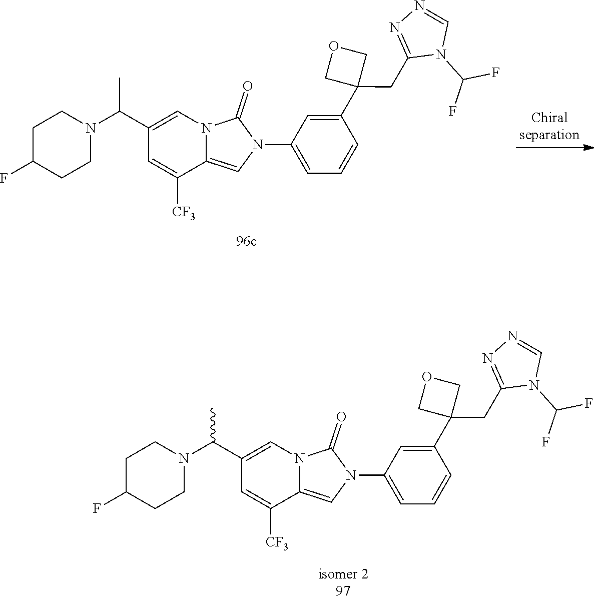 [SMILES: CC(c1cc(C(F)(F)F)c2cn(-c3cccc(C4(Cc5nncn5C(F)F)COC4)c3)c(=O)n2c1)N1CCC(F)CC1 ; CC(c1cc(C(F)(F)F)c2cn(-c3cccc(C4(Cc5nncn5C(F)F)COC4)c3)c(=O)n2c1)N1CCC(F)CC1]

### Synthesis of 97

96c (300 mg) was purified by chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 50% B to 50% B in 16.5 min; Wave Length: 220/254 urn; RT1 (min): 10.56, RT2 (min): 13.76, the first peak is product) to afford Compound 97 (102.5 mg, 33.82%) as a yellow solid.

LC-MS: (ES, m/z): [M+H]⁺ 595

H-NMR: (400 MHz, DMSO-d6, ppm): δ 1.26-1.27 (d, 1H), δ 1.68-1.81 (m, 2H), δ 1.82-1.86 (m, 2H), δ 2.33-2.36 (m, 2H), δ 2.51-2.54 (m, 2H), δ 3.50-3.51 (m, 1H), δ 3.75 (s, 2H), δ 4.60-4.72 (m, 1H), δ 4.96 (s, 4H), δ 7.06-7.08 (d, 2H), δ 7.33 (s, 1H), δ 7.39-7.43 (m, 1H), δ 7.54-7.61 (m, 2H), δ 7.69 (s, 1H), δ 7.72-7.75 (m, 1H), δ 8.84 (s, 1H).

### Example 98. Synthesis of Compound 98

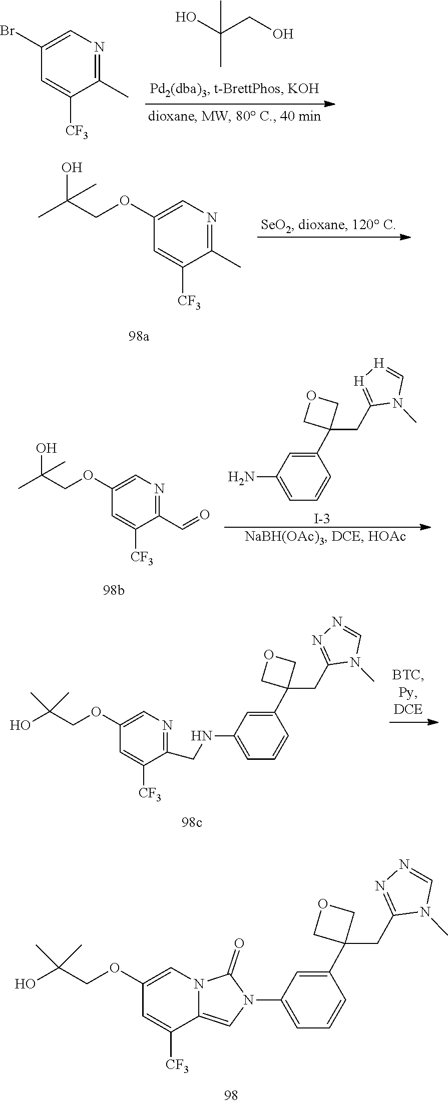

### Synthesis of 98a

To a stirred solution of 5-bromo-2-methyl-3-(trifluoromethyl) pyridine (2 g, 8.33 mmol, 1.0 equiv) and 2-methyl-propane-1,2-diol (4 mL) in 1,4-dioxane (20 ml) were added Pd₂(dba)₃ (763 mg, 0.83 mmol, 0.1 equiv) and t-BuBrettPhos (807 mg, 1.66 mmol, 0.2 equiv) at room temperature. To the above mixture was added KOH (1.4 g, 24.99 mmol, 3.0 equiv) in three portions over 2 min at room temperature. The final reaction mixture was irradiated with microwave radiation for 40 min at 80° C. The reaction was quenched with sat. NH₄Cl (aq.) (30 ml) at room temperature. The resulting mixture was extracted with CH₂Cl₂ (3×50 mL). The combined organic layers were washed with water (3×50 mL), dried over anhydrous Na₂SO₄. After filtration, the filtrate was concentrated under reduced pressure. The residue was purified by Prep-TLC (CH₂Cl₂/MeOH=10:1) to afford 98a (260 mg, 13%) as a yellow oil.

### Synthesis of 98b

A solution of 98a (240 mg, 0.96 mmol, 1.0 equiv) and SeO₂ (641 mg, 5.77 mmol, 6 equiv) in 1,4-dioxane (5 ml) was stirred overnight at 120° C. The reaction was quenched with water (30 ml) at room temperature. The resulting mixture was extracted with CH₂Cl₂ (3×20 mL). The combined organic layers were washed with water (3×20 mL) and dried over anhydrous CaCl₂). After filtration, the filtrate was concentrated under reduced pressure. The residue was purified by Prep-TLC (CH₂Cl₂/MeOH=50:1) to afford 98b (200 mg, 79%) as a yellow oil.

### Synthesis of 98c

To a stirred solution of 98b (190 mg, 0.72 mmol, 1.0 equiv) and I-3 (264 mg, 1.08 mmol, 1.5 equiv) in DCE (3 ml) were added HOAc (43 mg, 0.72 mmol, 1.0 equiv) and NaBH(OAc)₃ (458 mg, 2.16 mmol, 3.0 equiv) at room temperature. The resulting mixture was stirred overnight at room temperature. The reaction was quenched with sat. NH₄Cl (aq.) (20 ml) at room temperature. The resulting mixture was extracted with CH₂Cl₂ (3×20 mL). The combined organic layers were washed with water (3×10 mL) then dried over anhydrous CaCl₂. After filtration, the filtrate was concentrated under reduced pressure. The residue was purified by Prep-TLC (CH₂Cl₂/MeOH=10:1) to afford 98c (170 mg, 44%) as a yellow solid.

### Synthesis of 98

To a stirred solution of 98c (160 mg, 0.32 mmol, 1.0 equiv) and Pyridine (154 mg, 1.95 mmol, 6.0 equiv) in DCE (3 ml) was added triphosgene (34 mg, 0.11 mmol, 0.35 equiv) at 0° C. under nitrogen atmosphere. The resulting mixture was stirred for 10 min at room temperature under nitrogen atmosphere. The resulting mixture was washed with 3×10 mL of sat. NaHCO₃ (aq.) (10 ml). The resulting mixture was extracted with CH₂Cl₂ (3×10 mL). The combined organic layers were washed with water (3×10 mL) and dried over anhydrous CaCl₂). After filtration, the filtrate was concentrated under reduced pressure. The residue was purified by Prep-TLC (CH₂Cl₂/MeOH=10:1) to afford Compound 98 (61 mg, 35%) as a yellow solid,

LC-MS: (ES, m/z): [M+H]⁺ 518

H-NMR: (400 MHz, dmso-d6, δ ppm): 1.19 (s, 6H), 2.97 (s, 3H), 3.53 (s, 2H), 3.71 (s, 2H), 4.66 (s, 1H), 4.91-4.96 (m, 4H), 6.87-6.89 (d, 1H), 6.98 (s, 1H), 7.23 (s, 1H), 7.37-7.42 (m, 3H), 7.75-7.77 (m, 1H), 8.20 (s, 1H).

### Example 99. Synthesis of Compound 99

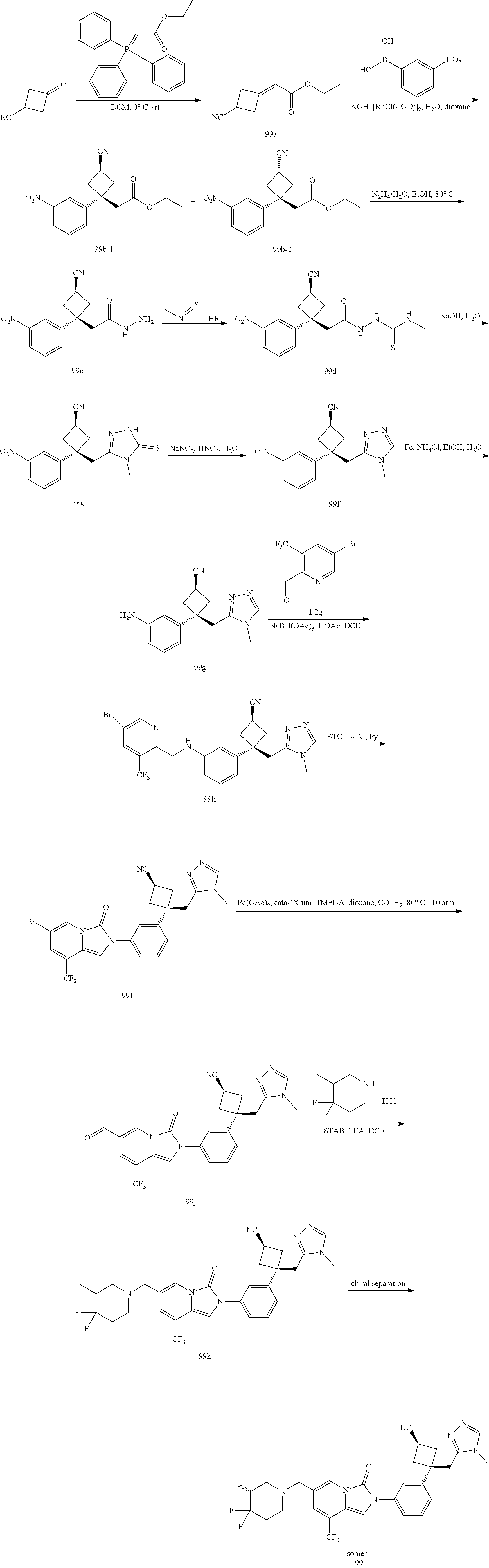

### Synthesis of 99a

To a stirred solution of 3-oxocyclobutane-1-carbonitrile (25 g, 262.878 mmol, 1 equiv) in DCM (500 mL) was added ethyl 2-(triphenyl-lambda5-phosphanylidene)acetate (137.37 g, 394,317 mmol, 1.5 equiv) at room temperature. The resulting mixture was stirred overnight at room temperature. The resulting mixture was filtered, the filter cake was washed with DCM (2×50 mL). The filtrate was concentrated under reduced pressure. The residue was purified by silica gel column chromatography, eluted with PE/EA (10:1) to afford 99a (25 g, 57.57%) as a colorless oil.

### Synthesis of 99b

A mixture of Chloro(1,5-cyclooctadiene)rhodium(I) Dimer (1 g, 2,028 mmol, 0.01 equiv) in dioxane (450 mL) and KOH (151.34 mL, 227.008 mmol, 1.5 equiv, 1M) was stirred for 1 h at room temperature under argon atmosphere. To the above mixture was added 3-nitrophenylboronic acid (50.53 g, 302.678 mmol, 2 equiv) and 99a (25 g, 151.339 mmol, 1 equiv). The resulting mixture was stirred overnight at room temperature. The reaction was quenched by the addition of saturated aqueous NH₄Cl (1500 mL). The resulting mixture was extracted with EtOAc (2×2 L). The combined organic layers concentrated under reduced pressure. The residue was purified by silica gel column chromatography, eluted with PE/EA (8:1) to afford 99b (2.1 g, 4.81%) as a light yellow solid; eluted with PB/EA (6:1) to afford 99b-2 (1.2 g, 2.75%) as a light yellow solid.

### Synthesis of 99c

To a stirred solution of 99b (2.1 g, 7.284 mmol, 1 equiv) in EtOH (10 mL) was added hydrazine hydrate (98%) (1.82 g, 36.420 mmol, 5 equiv). The resulting mixture was stirred overnight at 80° C. The resulting mixture was diluted with water (50 mL). The resulting mixture was extracted with CH₂Cl₂/MeOH=10/1 (3×50 mL). The combined organic layers were dried over anhydrous Na₂SO₄. After filtration, the filtrate was concentrated under reduced pressure. This resulted in 99c (1.3 g, 65.07%) as a light yellow solid.

### Synthesis of 99d

To a stirred solution of 99c (1.3 g, 4.740 mmol, 1 equiv) in tetrahydrofuran (20 mL) was added methyl isothiocyanate (0.69 g, 9.480 mmol, 2 equiv) at room temperature. The resulting mixture was stirred overnight at room temperature. The resulting mixture was diluted with water (60 mL). The resulting mixture was extracted with EtOAc (3×100 mL). The combined organic layers were concentrated under reduced pressure. This resulted in 99d (1.5 g, 91.10%) as a light yellow solid.

### Synthesis of 99e

To a stirred mixture of 99d (1.5 g, 4,318 mmol, 1 equiv) in H₂O (15 mL) was added NaOH (0.43 g, 10.751 mmol, 2.49 equiv) at room temperature. The resulting mixture was stirred for 4 h at room temperature. The mixture was acidified to pH 4 with HCl (aq.) (1M). The resulting mixture was extracted with CH₂Cl₂/MeOH=10/1 (3×30 mL). The combined organic layers were concentrated under reduced pressure. The resulting mixture was concentrated under reduced pressure. This resulted in 99e (1 g, 70.31%) as a yellow solid,

### Synthesis of 99f

To a stirred mixture of 99e (1 g, 3.036 mmol, 1 equiv) and NaNO₂ (2.09 g, 30,299 mmol, 9.98 equiv) in H₂O (10 mL) was added HNO₃ (30.36 mL, 30.360 mmol, 10 equiv, 1M) dropwise at 0° C. The resulting mixture was stirred overnight at room temperature. The reaction was quenched by the addition of NaHCO₃ (aq.) (20 mL) at room temperature. The resulting mixture was extracted with EtOAc (3×40 mL). The combined organic layers were dried over anhydrous Na₂SO₄. After filtration, the filtrate was concentrated under reduced pressure. This resulted in 99f (850 mg, 94.17%) as a light yellow solid.

### Synthesis of 99g

To a stirred mixture of 99f (850 mg, 2.859 mmol, 1 equiv) and NH₄HCl (764.62 mg, 14.295 mmol, 5 equiv) in EtOH (20 mL) and H₂O (5 mL) was added Fe (478.96 mg, 8.577 mmol, 3 equiv). The resulting mixture was stirred for 3 h at 80° C. The resulting mixture was diluted with water (80 mL). The resulting mixture was extracted with CH₂Cl₂ (3×100 mL). The combined organic layers were dried over anhydrous Na₂SO₄. After filtration, the filtrate was concentrated under reduced pressure. This resulted in 99g (800 mg) as a yellow solid.

### Synthesis of 99h

To a stirred solution of 99g (800 mg, 2,992 mmol, 1 equiv), HOAc (179.70 mg, 2.992 mmol, 1 equiv) and I-2g (1140.17 mg, 4.488 mmol, 1.5 equiv) in DCE (20 mL) was added NaBH(OAc)3 (1268.46 mg, 5.984 mmol, 2 equiv). The resulting mixture was stirred overnight at room temperature. The resulting mixture was diluted with water (20 mL). The resulting mixture was extracted with CH₂Cl₂ (3×25 mL). The combined organic layers were concentrated under reduced pressure. The residue was purified by trituration with methyl tert-butyl ether (10 mL). This resulted in 99h (1.3 g, 85.97%) as a light yellow solid.

### Synthesis of 99i

To a stirred solution of 99h (1.3 g, 2.573 mmol, 1 equiv) and Pyridine (1.22 g, 15.424 mmol, 6.00 equiv) in DCM (30 mL) was added Triphosgene (0.31 g, 1.029 mmol, 0.4 equiv) at 0° C. The resulting mixture was stirred for 30 min at 0° C. The reaction was quenched by the addition of saturated aqueous NaHCO₃ (20 mL). The resulting mixture was extracted with CH₂Cl₂/MeOH=10/1 (3×20 mL). The combined organic layers were dried over anhydrous Na₂SO₄, After filtration, the filtrate was concentrated under reduced pressure. The residue was purified by trituration with methyl tert-butyl ether (10 mL). This resulted in 99i (1 g, 73.16%) as a yellow solid.

### Synthesis of 99j

To a solution of 99i (1 g, 1,882 mmol, 1 equiv) in dioxane (30 mL) and TMEDA (0.44 g, 3.764 mmol, 2 equiv) was added bis(adamantan-1-yl)(butyl)phosphane (0.13 g, 0.376 mmol, 0.2 equiv) and Pd(OAc)2 (0.04 g, 0.188 mmol, 0.1 equiv) in an autoclave. After flushing the autoclave three times with CO/12 (1:1), the mixture was pressurized to 10 atm with CO/H₂ (1:1) at 80 degrees C. overnight. The resulting mixture was concentrated under vacuum. The residue was purified by silica gel column chromatography, eluted with CH₂Cl₂/MeOH (20:1) to CH₂Cl₂/MeOH (5:1) to afford 99j (550 mg, 60.83%) as a yellow solid.

### Synthesis of 99k

To a stirred mixture of 99j (300 mg, 0.624 mmol, 1 equiv) and 4,4-difluoro-3-methylpiperidine hydrochloride (321.49 mg, 1.872 mmol, 3 equiv) in DCE (10 mL) was added TEA (252.74 mg, 2.496 mmol, 4 equiv). The resulting mixture was stirred for 1h at room temperature. To the above mixture was added STAB (264.68 mg, 1.248 mmol, 2 equiv). The resulting mixture was stirred overnight at room temperature. The reaction was quenched by the addition of Water (10 mL). The resulting mixture was extracted with CH₂Cl₂/MeOH=10/1 (2×20 mL). The combined organic layers were concentrated under reduced pressure. The residue was purified by reverse flash chromatography with the following conditions: column, C18 silica gel; mobile phase, MeCN in water (10 mmoL NH₄HCO₃), 15% to 75% gradient in 30 min; detector, UV 254 nm. This resulted in 99k (100 mg, 26.71%) as a yellow solid.

### Synthesis of 99

99k (100 mg) was purified by chiral separation with the following conditions (Column: CHIRALPAK IC, 2*25 cm, 5 μm; Mobile Phase A: Hex (0.5% 2M NH3-MeOH), Mobile Phase B: MeOH:DCM=1:1 (0.1% 2M NH3-MEOH); Flow rate: 20 mL/min; Gradient: 50% B to 50% B in 14.5 min; Wave Length: 220/254 nm; RT1 (min): 12.07, RT2 (min): 13.38, the first peak is product) to afford Compound 99 (37.2 mg, 37.20%) as a yellow solid.

LC-MS: (ES, m/z): [M+H]⁺ 600

H-NMR: (400 MHz, DMSO-d6, ppm): δ 0.93-0.94 (d, 3H), δ 1.90-2.21 (m, 411), δ 2.25-2.36 (m, 1H), δ 2.72-2.95 (m, 9H), δ 3.22-3.24 (m, 1H), δ 3.35-3.36 (m, 4H), δ 6.94-6.96 (d, 1H), δ 7.04 (s, 1H), δ 7.32-7.33 (d, 1H), δ 7.40-7.45 (m, 2H), δ 7.71-7.74 (m, 2H), δ 8.18 (s, 1H).

### Example 100. Synthesis of Compound 100

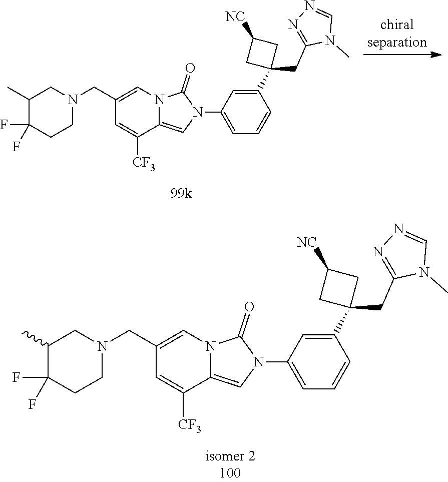 [SMILES: CC1CN(Cc2cc(C(F)(F)F)c3cn(-c4cccc([C@]5(Cc6nncn6C)C[C@@H](C#N)C5)c4)c(=O)n3c2)CCC1(F)F ; CC1CN(Cc2cc(C(F)(F)F)c3cn(-c4cccc([C@]5(Cc6nncn6C)C[C@@H](C#N)C5)c4)c(=O)n3c2)CCC1(F)F]

### Synthesis of 100

99k (100 mg) was purified by chiral separation with the following conditions (Column: CHIRALPAK IC, 2*25 cm, 5 μm; Mobile Phase A: Hex (0.5% 2M NH₃-MeOH), Mobile Phase B: MeOH:DCM=1:1 (0.1% 2M NH₃-MEOH); Flow rate: 20 mL/min; Gradient: 50% B to 50% B in 14.5 min; Wave Length: 220/254 nm; RT1 (min): 12.07, RT2 (min): 13.38, the second peak is product) to afford Compound 100 (38.6 mg, 38.00%) as a yellow solid.

LC-MS: (ES, m/z): [M+H]⁺ 600

H-NMR: (400 MHz, DMSO-d6, ppm): δ 0.93-0.94 (d, 3H), δ 1.90-2.20 (m, 411), δ 2.25-2.31 (m, 1H), δ 2.74-2.93 (m, 9H), δ 3.22-3.24 (m, 1H), δ 3.32 (s, 2H), δ 3.35 (s, 2H), δ 6.93-6.95 (d, 1H), δ 7.03 (s, 1H), δ 7.33 (s, 1H), δ 7.37-7.44 (m, 2H), δ7.72-7.75 (m, 2H), δ8.19 (s, 1H).

### Example 101 Synthesis of Compound 101

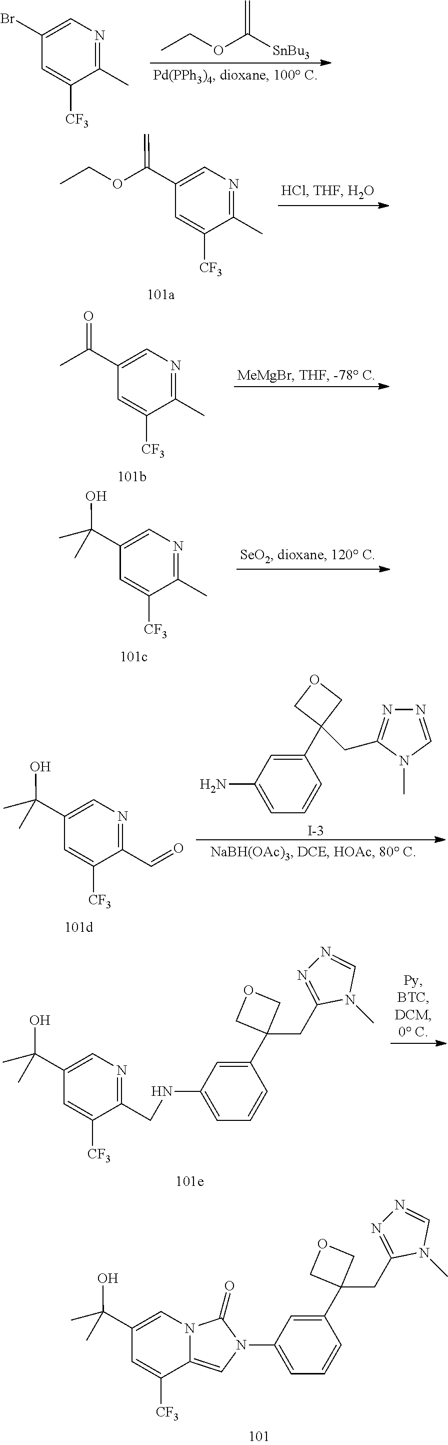

### Synthesis of 101a

Into a 8 mL vial were added 5-bromo-2-methyl-3-(trifluoromethyl)pyridine (500 mg, 2.083 mmol, 1 equiv), 1,4-dioxane (5 mL), tributyl(1-ethoxyethenyl)stannane (1504.68 ng, 4,166 mmol, 2 equiv), and Pd(PPh₃)4 (240.72 mg, 0.208 mmol, 0.1 equiv) at room temperature. The resulting mixture was stirred overnight at 100° C. under nitrogen atmosphere. The resulting mixture was concentrated under vacuum. The residue was purified by Prep-TLC (PE/EA 1:1) to afford 101a (400 mg, 83.05%) as a white solid.

### Synthesis of 101b

Into a 8 mL vial were added 101a (400 mg, 1,730 mmol, 1 equiv), THF (2 mL), HCl (2 mL, 2M), and H₂O (2 mL) at room temperature. The resulting mixture was stirred overnight at room temperature under nitrogen atmosphere. The resulting mixture was concentrated under vacuum. The residue was purified by Prep-TLC (PE/EA 1:1) to afford 101b (350 mg, 99.58%) as a white solid.

### Synthesis of 101c

Into a 25 mL 3-necked round-bottom flask were added 101b (500 mg, 2,461 mmol, 1 equiv) and THF (5 mL) at room temperature. To the above mixture was added MeMgBr (2.7 mL, 2.707 mmol, 1.1 equiv, 1M in THF) at −78° C. The resulting mixture was stirred for additional 1 h at −78° C. The reaction was quenched by the addition of sat. NH_{A}Cl (aq.) (10 mL) at −78° C. The aqueous layer was extracted with EtOAc (3×10 mL). The resulting mixture was concentrated under vacuum. The residue was purified by Prep-TLC (PE/EA 1:1) to afford 101c (300 mg, 55.61%) as a white solid.

### Synthesis of 101d

Into a 8 mL vial were added 101c (300 mg, 1,369 mmol, 1 equiv), dioxane (5 mL), and SeO₂ (759.28 mg, 6.845 mmol, 5 equiv) at room temperature. The resulting mixture was stirred overnight at 110° C. under nitrogen atmosphere. The reaction was quenched by the addition of water (10 mL) at room temperature. The aqueous layer was extracted with EtOAc (3×10 mL). The residue was purified by Prep-TLC (PE/EA 1:1) to afford 101d (260 mg, 81.47%) as a white solid.

### Synthesis of 101e

Into a 8 mL vial were added 101d (260 mg, 1.115 mmol, 1 equiv), DCE (4 mL), 1-3 (299.62 mg, 1.227 mmol, 1.1 equiv), NaBH(OAc)₃ (708.92 mug, 3.345 mmol, 3 equiv), and HOAc (133.91 mg, 2.230 mmol, 2 equiv) at room temperature. The resulting mixture was stirred overnight at 80° C. under nitrogen atmosphere. The reaction was quenched by the addition of water (5 mL) at room temperature. The aqueous layer was extracted with EtOAc (3×5 mL). The resulting mixture was concentrated under vacuum. The residue was purified by Prep-TLC (CH₂Cl₂/MeOH 30:1) to afford 101e (200 ng, 38.87%) as a white solid,

### Synthesis of 101

Into a 20 mL vial were added 101e (100 mg, 0.217 mmol, 1.00 equiv), DCM (2 mL) and Pyridine (85.70 mg, 1.085 mmol, 5 equiv) at 0 degrees C. To the above mixture was added BTC (22.51 mg, 0,076 mmol, 0.35 equiv) at 0 degrees C. The resulting mixture was stirred for additional 1 h at room temperature. The reaction was quenched by the addition of water (5 mL) at room temperature. The aqueous layer was extracted with EtOAc (3×5 mL). The resulting mixture was concentrated under vacuum. The residue was purified by reverse flash chromatography with the following conditions: column, C18 silica gel; mobile phase A: water, mobile phase B: CH₃CN, 10% B to 100% B gradient in 30 min; detector, LV 254 nm to afford Compound 101 (50.7 mug 70.02%) as a white solid.

LC-MS: (ES, m/z): [M+H]⁺ 488

H-NMR: (400 MHz, DMSO-d₆, ppm, δ): 1.41 (s, 6H), 2.97 (s, 3H), 3.53 (s, 2H), 4.91-4.96 (m, 4H), 5.29 (s, 1H), 6.89-6.91 (d, 1H), 7.20 (s, 1H), 7.31 (s, 1H), 7.38-7.42 (m, 2H), 7.65 (s, 1H), 7.75-7.77 (m, 1H), 8.20 (s, 1H).

### Example 102. Synthesis of Compound 102

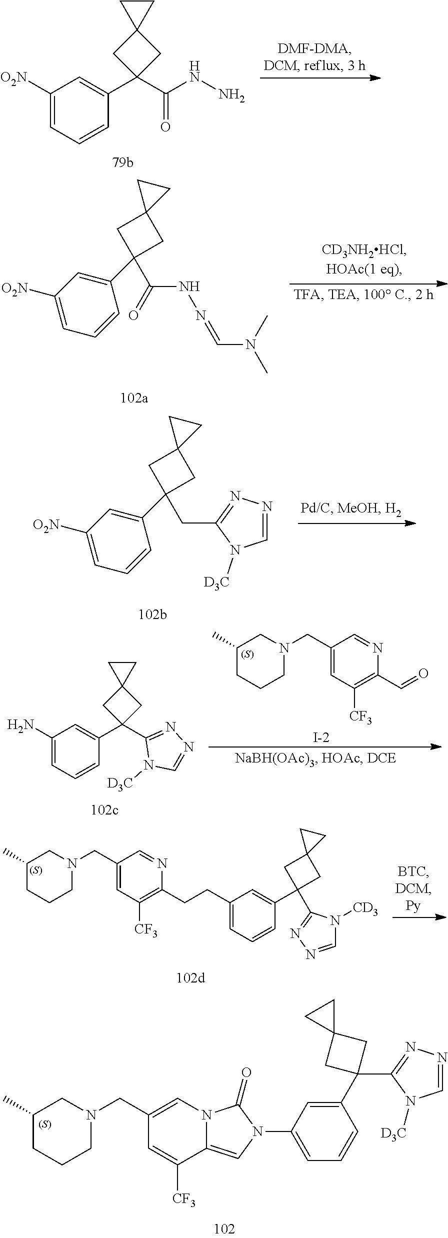

### Synthesis of 102a

Into a 100 mL round-bottom flask were added 79b (3 g, 11.482 mmol, 1 equiv) and DMF-DMA (2.74 g, 22.964 mmol, 2 equiv) in DCM (50 ml) at room temperature. The resulting mixture was stirred for 8 h at 50° C. under nitrogen atmosphere. The mixture was allowed to cool down to room temperature. The resulting mixture was concentrated under reduced pressure. The residue was purified by silica gel column chromatography, eluted with CH₂Cl₂/MeOH (12:1) to afford 102a (3 g, 75.98%) as a white solid.

### Synthesis of 102b

Into a 100 mL 3-necked round-bottom flask were added Methyl-d3-amine hydrochloride (2.23 g, 31,610 mmol, 5 equiv) and TEA (1.92 g, 18,966 mmol, 3 equiv) in THF (50 mL) at room temperature. The resulting mixture was stirred for 30 min at room temperature. To the above mixture were added 102a (2 g, 6.322 mmol, 1 equiv) and HOAc (0.38 g, 6.322 mmol, 1 equiv) at room temperature under nitrogen atmosphere. The resulting mixture was stirred for 3 days at 100° C. under nitrogen atmosphere. The mixture was allowed to cool down to room temperature. The reaction was diluted with water (150 mL). The aqueous layer was extracted with EtOAc (3×50 mL). The resulting mixture was concentrated under reduced pressure. The residue was purified by silica gel column chromatography, eluted with PE/EA (3:1) to 102b (500 mg, 24.67%) as a white solid.

### Synthesis of 102c

To a solution of 102b (500 mg, 1.659 mmol, 1 equiv) in MeOH (20 mL) was added Pd/C (50 mg) under nitrogen atmosphere in a 50 round-bottom flask. The mixture was hydrogenated at room temperature for 3h under hydrogen atmosphere using a hydrogen balloon, filtered through a Celite pad and concentrated under reduced pressure. This resulted in 102c (200 mg) as a yellow solid.

### Synthesis of 102d

To a stirred solution of 102c (180 mg, 0.699 mmol, 1 equiv) and 1-2 (200.24 mg, 0.699 mmol, 1 equiv) in DCE (5 mL) were added HOAc (42.00 mg, 0.699 mmol, 1 equiv) and NaBH(OAc)3 (222.35 mg, 1.048 mmol, 1.5 equiv). The resulting mixture was stirred overnight at room temperature. The reaction was quenched by the addition of water (10 mL). The resulting mixture was extracted with CH₂Cl₂ (3×10 mL). The combined organic layers were concentrated under reduced pressure. The residue was purified by Prep-TLC (CH₂Cl₂/MeOH 20:1) to afford 102d (276 mg, 74.79%) as a white solid.

### Synthesis of 102

To a stirred solution of 102d (256 mg, 0.485 mmol, 1 equiv) and Pyridine (230.26 mg, 2.910 mmol, 6 equiv) in DCM (10 mL) was added Triphosgene (57.59 mg, 0.194 mmol, 0.4 equiv) at 0° C. The resulting mixture was stirred for 1 h at 0° C. The reaction was quenched by the addition of NaHCO₃ (aq.) (10 mL). The resulting mixture was extracted with CH₂Cl₂ (3×10 mL). The combined organic layers were concentrated under reduced pressure. The residue was purified by Prep-TLC (CH₂Cl₂/MeOH 15:1) to afford Compound 102 (147.2 mg, 54.80%) as a yellow solid.

LC-MS: (ES, m/z): [M+H]⁺ 554

H-NMR: (400 MHz, DMSO-d6, ppm): δ 0.44-0.59 (m, 4H), δ 0.75-0.93 (m, 4H), δ 1.43-1.66 (m, 5H), δ 1.87-1.91 (m, 1H), δ 2.67-2.78 (m, 4H), δ 3.17-3.25 (m, 4H), δ 7.00 (s, 1H), δ 7.17-7.19 (d, 1H), δ 7.30-7.53 (m, 2H), δ 7.63-7.67 (m, 2H), δ 7.83-7.84 (d, 1H), 38.40 (s, 1H).

### Example 103. Synthesis of Compound 103

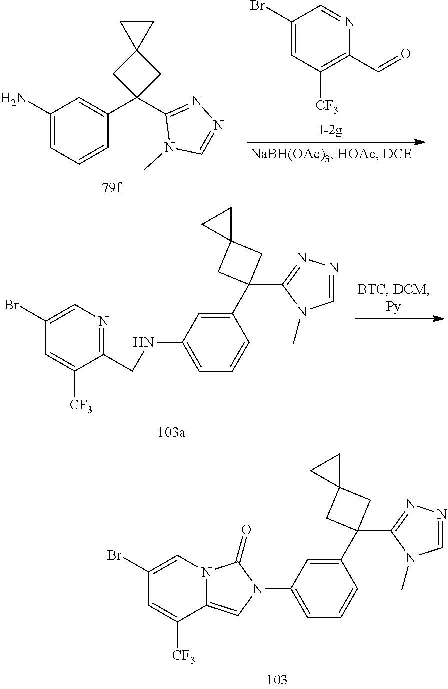 [SMILES: Cn1cnnc1C1(c2cccc(N)c2)CC2(CC2)C1 ; O=Cc1ncc(Br)cc1C(F)(F)F ; Cn1cnnc1C1(c2cccc(NCc3ncc(Br)cc3C(F)(F)F)c2)CC2(CC2)C1 ; Cn1cnnc1C1(c2cccc(-n3cc4c(C(F)(F)F)cc(Br)cn4c3=O)c2)CC2(CC2)C1]

### Synthesis of 103a

To a stirred mixture of 79f (200 mg, 0,786 mmol, 1 equiv) and I-2g (239.69 mg, 0.943 mmol, 1.2 equiv) in DCE (2 mL) were added HOAc (47.22 mg, 0.786 mmol, 1 equiv) and NaBH(OAc)₃ (499.98 mg, 2.358 mmol, 3 equiv) at room temperature. The resulting mixture was stirred overnight at room temperature. The reaction was quenched by the addition of H₂O (10 mL) at room temperature. The aqueous layer was extracted with CH₂Cl₂ (2×10 mL). The resulting mixture was concentrated under vacuum. The residue was purified by Prep-TLC (CH₂Cl₂/MeOH=20:1) to afford 103a (230 mg, 55.25%) as a yellow solid.

### Synthesis of 103

To a stirred solution of 103a (210 mg, 0.427 mmol, 1.00 equiv) and pyridine (202.43 mg, 2.562 mmol, 6 equiv) in DCM (5 mL) was added triphosgene (44.30 mg, 0.149 mmol, 0.35 equiv) at 0° C. The reaction was quenched by the addition of saturated aqueous NaHCO₃ (20 mL) at room temperature. The aqueous layer was extracted with CH₂Cl₂ (2×5 mL). The resulting mixture was concentrated under reduced pressure. The residue was purified by Prep-TLC (CH₂Cl₂/MeOH=15:1) to afford Compound 103 (107.4 mg, 48.29%) as a yellow solid.

LCMS: (ES, m/z): [M+H]⁺ 518.

H-NMR: (400 MHz, DMSO-d₆, ppm) δ 0.44-0.58 (m, 4H), 2.74-2.78 (d, 2H), 3.16-3.23 (m, 5H), 7.16-7.21 (m, 2H), 7.21-7.53 (m, 2H), 7.62-7.64 (d, 1H), 7.82 (s, 1H), 8.04 (s, 1H), 8.39 (s, 1H).

### Example 104. Synthesis of Compound 104

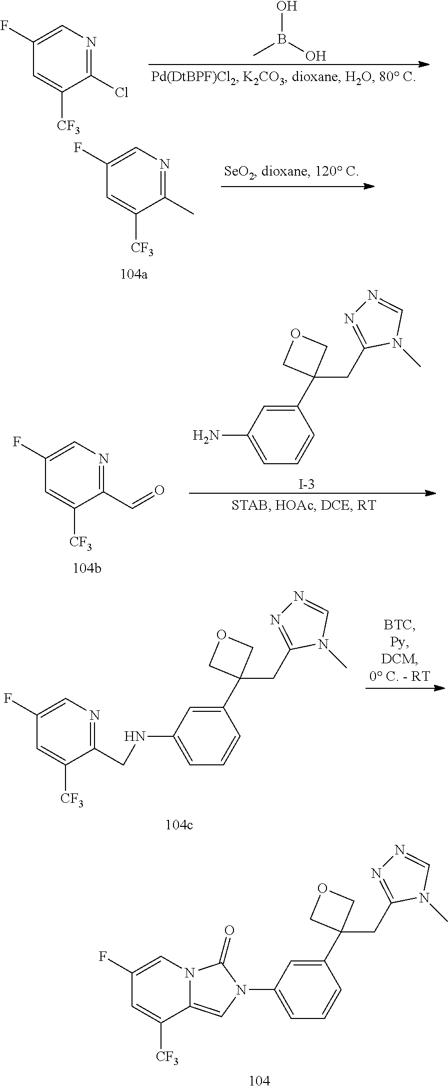

### Synthesis of 104a

To a stirred solution of 2-chlor-5-fluoro-3-(trifluoromethyl)pyridine (1 g, 5.012 mmol, 1.00 equiv) and methylboronic acid (0.90 g, 15.036 mmol 3 equiv) in dioxane (20 mL) was added K₂CO₃ (1.39 g, 10.024 mmol, 2 equiv), H₂O (4 mL), and Pd(DtBPF)Cl₂ (0.33 g, 0,501 mmol, 0.1 equiv) at room temperature under nitrogen atmosphere. The resulting mixture was stirred overnight at 80 degrees C. under nitrogen atmosphere. The residue was purified by silica gel column chromatography, eluted with PE to afford 104a (10 mL, product in dioxane) as a light yellow liquid.

### Synthesis of 104b

To a stirred solution of 104a (product in dioxane) (10 mL) was added SeO₂ (3.10 g, 27.910 mmol) at room temperature under nitrogen atmosphere. The resulting mixture was stirred overnight at 120 degrees C. The residue was purified by silica gel column chromatography, eluted with PE to afford 104b (80 mg, 12.62%) as a light yellow oil.

### Synthesis of 104c

To a stirred solution of 104b (70 mg, 0.363 mmol, 1.00 equiv) and I-3 (88.56 mg, 0.363 mmol, 1 equiv) in DCE (5 mL) was added STAB (230.49 mg, 1.089 mmol, 3 equiv) and HOAc (21.77 mg, 0.363 mmol, 1 equiv) at room temperature under nitrogen atmosphere. The resulting mixture was stirred for 3 h at room temperature. The reaction was quenched with saturated aqueous NH₄Cl at room temperature. The aqueous layer was extracted with CH₂Cl₂ (2×10 mL). The residue was purified by Prep-TLC (CH₂Cl₂/MeOH 12:1) to afford 104c (60 mg, 35.74%) as a light yellow oil.

### Synthesis of 104

To a stirred solution of 104c (50 mg, 0.119 mmol, 1.00 equiv) and Pyridine (187.71 mg, 2.380 mmol, 20 equiv) in DCM (10 mL) was added Triphosgene (35.21 mg, 0.119 mmol, 1 equiv) at 0 degrees C. under nitrogen atmosphere. The resulting mixture was stirred for 10 min at room temperature. The reaction was quenched by the addition of saturated aqueous NaHCO₃ (10 mL) at room temperature. The aqueous layer was extracted with CH₂Cl₂ (2×10 mL). The residue was purified by Prep-TLC (CH₂Cl₂/MeOH 12:1) to afford Compound 104 (17.7 mg, 32.68%) as a yellow solid.

LC-MS: (ES, m/z): [M+H]⁺ 448

H-NMR: (400 MHz, CD₃OD, δ ppm): 2.96 (s, 3H), 3.66 (s, 3H), 5.10 (s, 4H), 6.90-9.92 (d, 1H), 7.14-7.16 (m, 1H), 7.25 (s, 1H), 7.31-7.32 (d, 1H), 7.45-7.49 (m, 1H), 7.61-7.74 (m, 1H), 7.77-7.78 (d, 1H), 8.20 (s, 1H).

### Example 105. Synthesis of Compound 105

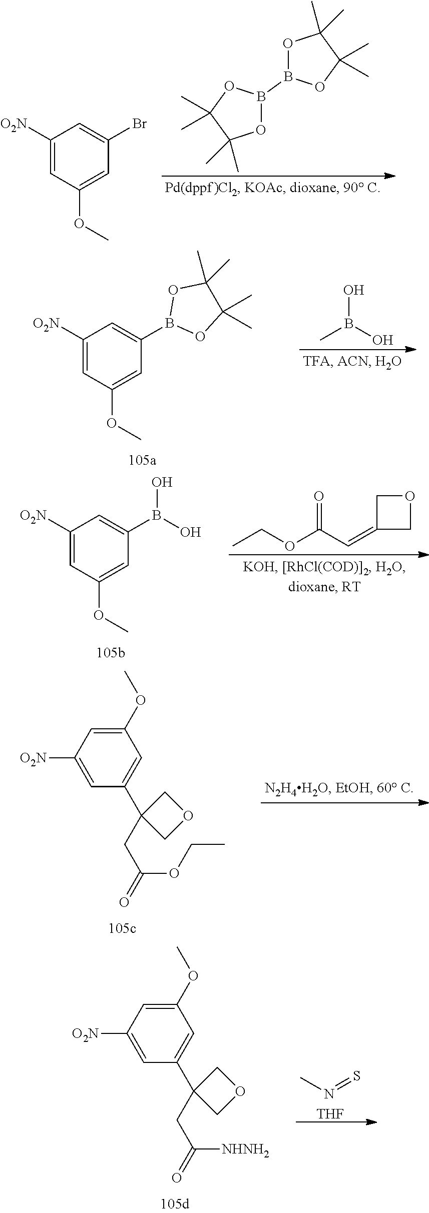

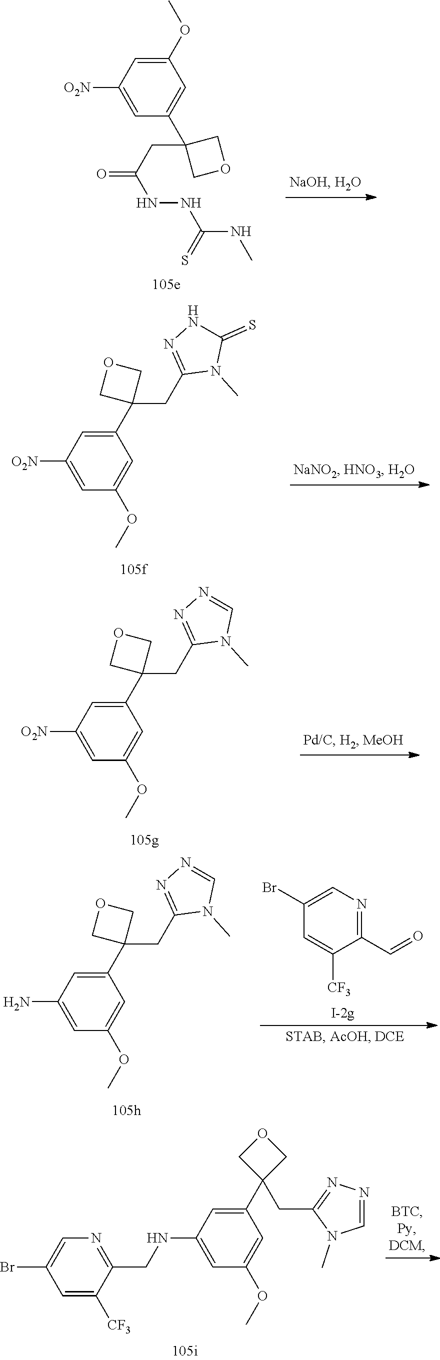 [SMILES: CNC(=S)NNC(=O)CC1(c2cc(OC)cc([N+](=O)[O-])c2)COC1 ; COc1cc([N+](=O)[O-])cc(C2(Cc3n[nH]c(=S)n3C)COC2)c1 ; COc1cc([N+](=O)[O-])cc(C2(Cc3nncn3C)COC2)c1 ; COc1cc(N)cc(C2(Cc3nncn3C)COC2)c1 ; O=Cc1ncc(Br)cc1C(F)(F)F ; COc1cc(NCc2ncc(Br)cc2C(F)(F)F)cc(C2(Cc3nncn3C)COC2)c1]

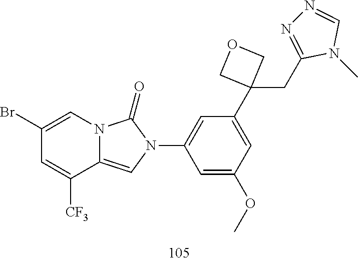 [SMILES: COc1cc(-n2cc3c(C(F)(F)F)cc(Br)cn3c2=O)cc(C2(Cc3nncn3C)COC2)c1]

### Synthesis of 10a

Into a 500 ml, 3-necked round-bottom flask were added 1-bromo-3-methoxy-5-nitrobenzene (20 g, 86.195 mmol, 1 equiv), dioxane (200 mL), KOAc (16.92 g, 172.390 mmol, 2 equiv), bis(pinacolato)diboron (43.78 g, 172.390 mmol, 2 equiv), and Pd(dppf)Cl₂ (1.26 g, 1.722 mmol, 0.02 equiv) at room temperature. The resulting mixture was stirred for 12 h at 80° C. under nitrogen atmosphere. The reaction was diluted with water (600 ML). The resulting mixture was extracted with EtOAc (3×300 mL). The combined organic layers were washed with water (3×10 mL) and dried over anhydrous Na₂SO₄. After filtration, the filtrate was concentrated under reduced pressure. The residue was purified by silica gel column chromatography, eluted with PE/EA (50:1) to afford 105a (25 g, 98.72%) as a light yellow solid.

### Synthesis of 105b

Into a 500 mL 3-necked round-bottom flask were added 105a (17 g, 60.910 mmol, 1 equiv), MeCN (200 mL), 1120 (20 mL), HCl (20 mL, 12 M) and methylboronic acid (18.23 g, 304.550 mmol, 5 equiv) at room temperature. The resulting mixture was stirred for 12 h at room temperature. The resulting mixture was concentrated under reduced pressure. The residue was purified by silica gel column chromatography, eluted with CH₂Cl₂/MeOH (10:1) to afford 105b (10 g, 80.03%) as a white solid.

### Synthesis of 105c

To a solution of Chloro(1,5-cyclooctadiene)rhodium(I) Dimer (1.25 g, 2,539 mmol, 0.05 equiv) in dioxane (150 mL) was added KOH (61 mL, 60.929 mmol, 1.2 equiv, 1 M) at room temperature under nitrogen atmosphere. The mixture was stirred for 30 min. 105b (10 g, 50:774 mmol, 1 equiv) and ethyl 2-(oxetan-3-ylidene)acetate (7.22 g, 50.774 mmol, 1 equiv) were added and the mixture was stirred at room temperature for 12 h under nitrogen atmosphere. The reaction mixture was quenched with NH₄Cl (aq.) (300 mL) and extracted with EtOAc (3×150 mL). The combined organic layers were dried over anhydrous Na₂SO₄. After filtration, the filtrate was concentrated under reduced pressure. The residue was purified by silica gel column chromatography, eluted with PE/EA (50:1) to afford 105C (5.9 g, 37.78%) as a light yellow solid.

### Synthesis of 105d

Into a 250 ml, 3-necked round-bottom flask were added 105c (4.8 g, 16.255 Mmol, 1 equiv), EtOH (50 mL), and hydrazine (8.12 g, 162.550 mmol, 10 equiv) at room temperature. The resulting mixture was stirred for 12 h at 80° C. The resulting mixture was diluted with water (300 mL) and extracted with EtOAc (3×200 mL). The combined organic layers were dried over anhydrous Na₂SO₄. After filtration, the filtrate was concentrated under reduced pressure. This resulted in 105d (4.8 g, 100.79%) as a light yellow oil.

### Synthesis of 105e

Into a 250 ml, 3-necked round-bottom flask were added 105d (4.8 g, 17.066 mmol, 1 equiv), tetrahydrofuran (50 mL), and methyl isothiocyanate (3.12 g, 42.665 mmol, 2.5 equiv) at room temperature. The resulting mixture was stirred for 12 h at room temperature under nitrogen atmosphere. The reaction was quenched with water (150 mL) at room temperature. The precipitated solids were collected by filtration and washed with water (3×10 mL). The resulting solid was dried under vacuum. This resulted in 105e (53 g, 81.50%) as a white solid.

### Synthesis of 105f

To a stirred mixture of 105e (53 g, 14.956 mmol, 1 equiv) in H₂O (110 mL) was added NaOH (4.79 g, 119.648 mmol, 8 equiv). The resulting mixture was stirred overnight at room temperature. The mixture was acidified to pH 4 with HCl (aq, 1M). The precipitated solids were collected by filtration and washed with water (3×15 mL) to afford 105f (7 g, crude) as a yellow solid.

### Synthesis of 105g

To a stirred mixture of 105f (6.9 g, 20.513 mmol, 1 equiv) and NaNO₂ (14.15 g, 205.130 mmol, 10 equiv) in H₂O (70 mL) was added H NO₃ (205 mL, 205.130 mmol, 10 equiv) dropwise at 0° C. The resulting mixture was stirred for 1h at 0° C. The reaction was quenched with NaHCO₃ (aq.) (100 mL) at room temperature. The aqueous layer was extracted with CH₂Cl₂ (3×200 mL). The resulting mixture was concentrated under reduced pressure to afford 105g (3.3 g, 49.16%) as a yellow solid.

### Synthesis of 105h

To a solution of 105g (3.2 g, 10.516 mmol, 1 equiv) in 100 mL MeOH was added Pd/C (10%, 300 mg) under nitrogen atmosphere. The mixture was hydrogenated at room temperature overnight under hydrogen atmosphere using a hydrogen balloon, filtered through a Celite pad and concentrated under reduced pressure. This resulted in 105h (1.5 g, 78.00%) as a yellow solid.

### Synthesis of 105i

To a stirred solution of 105h (600 mg, 2,187 mmol, 1 equiv) and I-2g (833.34 mg, 3.280 mmol, 1.5 equiv) in DCE (6 mL) were added STAB (927.11 mg, 4.374 mmol, 2 equiv) and AcOH (131.35 mg, 2.187 mmol, 1 equiv). The resulting mixture was stirred overnight at room temperature. The resulting mixture was diluted with water (20 mL). The aqueous layer was extracted with CH₂Cl₂ (3×20 mL). The resulting mixture was concentrated under reduced pressure. The residue was purified by Prep-TLC (CH₂Cl₂/MeOH 12:1) to afford 105i (626 mg, 51.95%) as a yellow solid.

### Synthesis of 105

To a stirred solution of 105i (620 mg, 1.210 mmol, 1 equiv) and Pyridine (574.34 mg, 7.260 mmol, 6 equiv) in DCM (20 mL) was added Triphosgene (143.64 mg, 0.484 mmol, 0.4 equiv) at 0° C. The resulting mixture was stirred for 2 h at 0° C. The reaction was quenched by the addition of NaHCO₃ (aq.) (20 mL). The resulting mixture was extracted with CH₂Cl₂ (3×30 mL). The combined organic layers were concentrated under reduced pressure. The residue was purified by Prep-TLC (CH₂Cl₂/MeOH 15:1) to afford Compound 105 (460 mg, 70.61%) as a yellow solid.

LC-MS: (ES, m/z): [M+H]⁺ 538

H-NMR: (400 MHz, DMSO-d6, ppm): δ 2.97 (s, 3H), δ 3.52 (s, 2H), δ 3.71 (s, 2H), δ 4.88-4.93 (m, 4H), δ 6.40-6.41 (d, 1H), δ 7.02 (s, 1H), δ 7.17 (s, 1H), δ 7.32-7.34 (d, 1H), δ 7.45 (s, 1H), δ 8.02 (s, 1H), δ 8.21 (s, 1H).

### Example 106. Synthesis of Compound 106

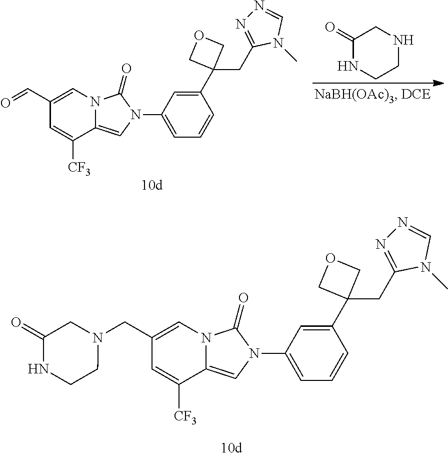 [SMILES: Cn1cnnc1CC1(c2cccc(-n3cc4c(C(F)(F)F)cc(C=O)cn4c3=O)c2)COC1 ; O=C1CNCCN1 ; Cn1cnnc1CC1(c2cccc(-n3cc4c(C(F)(F)F)cc(CN5CCNC(=O)C5)cn4c3=O)c2)COC1]

### Synthesis of 106

To a stirred solution of 10d (150 mg, 0.328 mmol, 1.00 equiv) and piperazin-2-one (98.50 mg, 0,984 mmol, 3 equiv) in DCE (2 ml) were added HOAc (19.69 mg, 0.328 mmol, 1 equiv) and NaBH(OAc)₃ (139.00 mg, 0.656 mmol, 2 equiv). The resulting mixture was stirred overnight at room temperature. The resulting mixture was concentrated under reduced pressure. The residue was purified by Prep-HPLC with the following conditions (Column: YMC-Actus Triart C18 ExRS, 30*150 mm, Sp-m; Mobile Phase A: Water (10 mmol/L NH₄HCO₃), Mobile Phase B: ACN; Flow rate: 60 mL/min; Gradient: 10% B to 36% B in 8 min, Wave Length: 220 nm; RT1 (min): 7.90) to afford Compound 106 as a yellow solid.

LC-MS: (ES, m/z): [M+H]⁺ 542

H-NMR: (400 MHz, DMSO-d6, ppm): δ 2.67-2.68 (m, 2H), δ 2.86-3.02 (m, 5H), δ 3.10-3.16 (m, 2H), δ 3.43 (s, 2H), δ 3.53 (s, 2H), δ 4.87-4.96 (m, 4H), δ 6.88-6.90 (m, 1H), δ 7.04 (s, 1H), δ 7.33 (s, 1H), δ 7.38-7.42 (m, 2H), δ 7.74-7.77 (m, 3H), δ 8.20 (s, 1H).

### Example 107. Synthesis of Compound 107

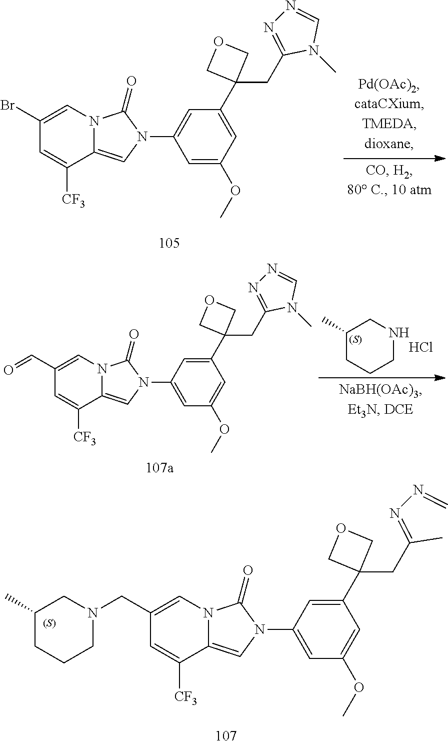

### Synthesis of 107a

To a solution of 105 (3 g, 5.573 mmol, 1 equiv) and TMEDA (1.30 g, 11.146 mmol, 2 equiv) in dioxane (90 mL) was added bis(adamantan-1-yl)(butyl)phosphane (0.40 g, 1.115 mmol, 0.2 equiv) and Pd(OAc)2 (0.13 g, 0.557 mmol, 0.1 equiv) in an autoclave. After flushing the autoclave three times with CO/H₂ (1:1), the mixture was pressurized to 10 atm with CO/H₂ (1:1) at 80 degrees C. overnight. The resulting mixture was concentrated under vacuum. The residue was purified by silica gel column chromatography, eluted with CH₂Cl₂/MeOH (20:1) to CH₂Cl₂/MeOH (5:1) to afford 107a (1.5 g, 55.22%) as a yellow solid.

### Synthesis of 107

To a stirred mixture of 107a (200 mg, 0.410 mmol, 1 equiv) and (3S)-3-methylpiperidine hydrochloride (166.96 mg, 1.230 mmol, 3 equiv) in DCE (10 mL) was added Et₃N (166.08 mg 1.640 mmol, 4 equiv) and NaBH(OAc)3 (173.92 mg, 0.820 mmol, 2 equiv). The resulting mixture was stirred overnight at room temperature. The resulting mixture was diluted with water (10 mL). The resulting mixture was extracted with CH₂Cl₂ (3×20 mL). The combined organic layers were concentrated under reduced pressure. The residue was purified by Prep-TLC (CH₂Cl₂/MeOH 15:1) to afford Compound 107 (64.1 mg, 27.38%) as a yellow solid.

LC-MS: (ES, m/z): [M+H]⁺ 414

H-NMR: (400 MHz, DMSO-d6, ppm): δ 0.82-0.86 (m, 4H), δ 1.46-1.58 (m, 1H), δ 1.62-1.66 (m, 4H), δ 1.87-1.92 (m, 1H), δ 2.72-2.77 (m, 2H), δ 2.99 (s, 3H), δ 3.25 (s, 2H), δ 3.52 (s, 2H), δ 3.71 (s, 3H), δ 4.89-4.94 (m, 4H), δ 6.40 (s, 1H), δ 7.01 (s, 2H), δ 7.33 (s, 1H), δ 7.37-7.37 (d, 1H), δ 7.64 (s, 1H), δ 8.22 (s, 1H).

### Example 108. Synthesis of Compound 108

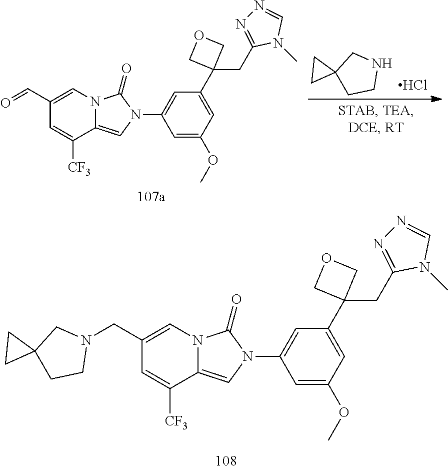

### Synthesis of 108

To a stirred solution of 107a (150 mg, 0.308 mmol, 1.00 equiv) and 5-azaspiro[2.4]heptane hydrochloride (123.36 mg, 0.924 mmol, 3 equiv) in DCE (5 mL) was added TEA (93.42 mg, 0.924 mmol, 3 equiv) at room temperature under nitrogen atmosphere. The resulting mixture was stirred for 1h at room temperature. To the above mixture was added STAB (130.44 mg, 0.616 mmol, 2 equiv) at room temperature. The resulting mixture was stirred for additional 3h at room temperature. The reaction was quenched by the addition of NH₄Cl (aq.) (20 mL) at room temperature. The aqueous layer was extracted with DCM (2×20 mL). The residue was purified by Prep-TLC (DCM/MeOH 20:1) to afford the crude product. The residue was purified by reverse flash chromatography with the following conditions: column, C18 silica gel; mobile phase A: water (10 mmol/L NaHCO₃), mobile phase B: MeCN, 0% B to 50% B gradient in 15 min; detector, UV 254 nm. This resulted in Compound 108 (31.3 mg, 17.33%) as a yellow solid.

LC-MS: (ES, m/z): [M+H]⁺ 569

H-NMR: (400 MHz, CD₃OD, δ ppm): 0.57-0.64 (m, 4H), 1.88-1.91 (m, 2H), 2.58 (s, 2H), 2.81-2.84 (m, 2H), 3.01 (s, 3H), 3.50 (s, 2H), 3.69 (s, 2H), 3.78 (s, 3H), 5.06 (s, 4H), 6.38 (m, 1H), 6.85-6.86 (m, 1H), 7.11 (s, 1H), 7.12-7.16 (m, 1H), 7.25-7.26 (m, 1H), 7.71 (s, 1H), 8.23 (s, 1H).

### Example 109. Synthesis of Compound 109

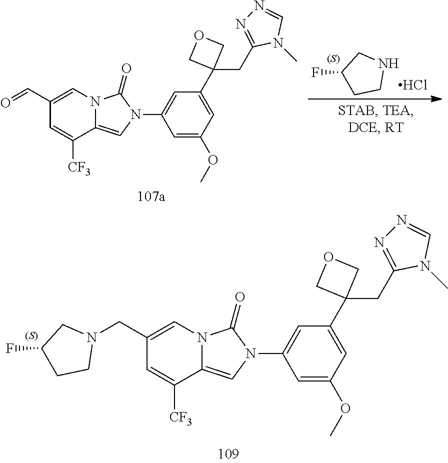

### Synthesis of 109

To a stirred solution of 107a (150 mg, 0.308 m-mol, 10 equiv) and (3S)-3-fluoropyrrolidine hydrochloride (115.93 mg, 0,924 mmol, 3 equiv) in DCE (5 mL) were added TEA (93.42 mg, 0.924 mmol, 3 equiv) at room temperature under nitrogen atmosphere. The resulting mixture was stirred for 1h at room temperature. To the above mixture was added STAB (130.44 mg, 0,616 mmol, 2 equiv) at room temperature. The resulting mixture was stirred for additional 3h at room temperature. The reaction was quenched by the addition of NH₄Cl (aq.) (20 mL) at room temperature. The aqueous layer was extracted with DCM (2×20 mL). The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 45% B in 7 min, Wave Length: 220 nm; RT1 (min): 6.70) to afford 109 (33.7 mg, 19.24%) as a yellow solid.

LC-MS: (ES, m/z): [M+H]⁺ 561

H-NMR: (400 MHz, CD₃OD, δ ppm): 1.99-2.11 (m, 1H), 2.18-2.25 (m, 1H), 2.47-2.51 (m, 1H), 2.69-2.81 (m, 1H), 2.89-2.98 (m, 2H), 3.01 (s, 3H), 3.51 (s, 2H), 3.66 (s, 2H), 3.78 (s, 3H), 5.06 (s, 4H), 5.12-5.26 (m, 1H), 6.38 (s, 1H), 6.85-6.86 (m, 1H), 7.11-7.13 (m, 2H), 7.25-7.26 (m, 1H), 7.70 (s, 1H), 8.23 (s, 1H).

### Example 110. Synthesis of Compound 110

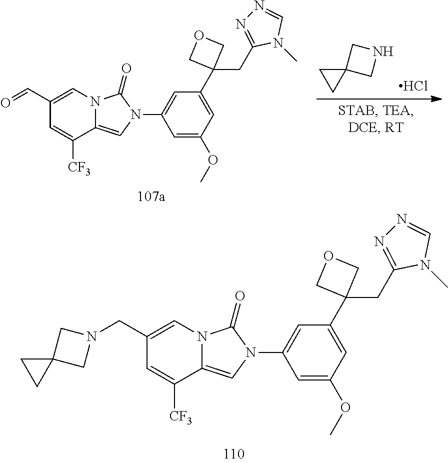

### Synthesis of 110

To a stirred solution of 107a (200 mg, 0.410 mmol, 1.00 equiv) and 5-azaspiro[2.3]hexane hydrochloride (147.21 mg, 1,230 mmol, 3 equiv) in DCE (5 mL) were added TEA (124.56 mg, 1.230 mmol, 3 equiv) at room temperature under nitrogen atmosphere. The resulting mixture was stirred for 1h at room temperature. To the above mixture was added STAB (173.92 mg 0.820 mmol, 2 equiv) at room temperature. The resulting mixture was stirred for additional 3h at room temperature. The reaction was quenched by the addition of saturated aqueous NH₄Cl (20n) at room temperature. The aqueous layer was extracted with DCM (2×20 mL). The residue was purified by Prep-TLC DCM/MeOH 20:1) to afford the crude product. The residue was purified by reverse flash chromatography with the following conditions: column, C18 silica gel; Mobile Phase A: Water (10 mmol/L NH₄HCO₃), Mobile Phase B: ACN; Gradient: 0% B to 50% B in 15 min; detector, UV 254 nm. This resulted in Compound 110 (95.0 mg, 40.87%) as a yellow solid.

LC-MS: (ES, m/z): [M+H]⁺ 555

H-NMR: (400 MHz, CD₃OD, δ ppm): 0.61 (s, 4H), 3.01 (s, 3H), 3.46 (s, 4H), 3.50 (s, 2H), 3.66 (s, 2H), 3.78 (s, 3H), 5.06 (s, 4H), 6.38-6.39 (m, 1H), 6.85-6.86 (m, 1H), 7.07 (s, 1H), 7.13 (s, 1H), 7.25-7.26 (m, 1H), 7.71 (s, 1H), 8.23 (s, 1H).

### Example 111. Synthesis of Compound 111

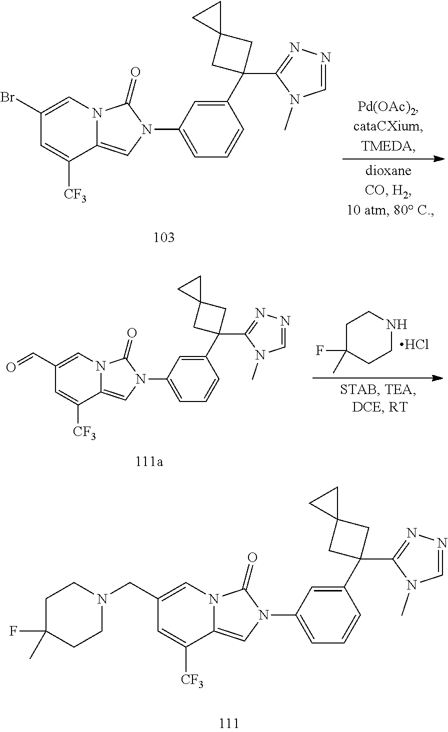

### Synthesis of 111a

To a mixture of 103 (4 g, 7.717 mmol, 1 equiv) in 400 ml, dioxane was added bis(adamantan-1-yl)(butyl)phosphane (0.55 g, 1.543 mmol, 0.2 equiv), Pd(AcO)₂ (0.17 g, 0,772 mmol, 0.1 equiv), TMEDA (1.79 g, 15.434 mmol, 2 equiv) in a pressure tank. The mixture was purged with nitrogen and then was pressurized to 10 atm with CO: H₂=1:1 and heated to 80° C. overnight. The reaction mixture was cooled to room temperature. The reaction was diluted with H₂O (1500 mL) at room temperature. The aqueous layer was extracted with EA (3×500 ml). The combined organic layers were concentrated under reduced pressure. The residue was purified by silica gel column chromatography, eluted with DCM/MeOH=50:1 to afford 111a (2.0 g, 55.44%) as a yellow solid.

### Synthesis of 111

To a stirred mixture of 111a (250 mg, 0.535 mmol, 1 equiv) and 4-fluoro-4-methylpiperidine hydrochloride (246.49 mg, 1.605 mmol, 3 equiv) in DCE (3.00 mL) were added TEA (162.35 mg, 1.605 mmol, 3 equiv) and NaBH(OAc)3 (226.70 mg, 1.070 mmol, 2 equiv) at room temperature. The resulting mixture was stirred overnight at room temperature. The reaction was quenched with sat NH₄Cl (aq.) (10 mL) at room temperature. The aqueous layer was extracted with EtOAc (3×10 mL). The resulting mixture was concentrated under reduced pressure. The residue was purified by Prep-TLC (DCM/MeOH 10:1) to afford 111 (250 mg, crude). The crude product (250 mg) was purified by Prep-HPLC with the following conditions (Column: XBridge Prep OBD C18 Column, 30*150 mm, 5 μm; Mobile Phase A: Water (10 mmol/I NH₄HCO₃), Mobile Phase B: ACN; Flow rate: 60 mL/min; Gradient: 41% B to 58% B in 8 min; Wave Length: 220 inn; RT1 (min): 7.72) to afford Compound 111 (79 mag, 25.98%) as yellow solid.

LC-MS: (ES, m/z): [M+H]⁺ 569

H-NMR: (400 MHz, DMSO, δ ppm): 0.44-0.55 (m, 2H), 0.56-0.59 (t, 2H), 1.29-1.34 (d, 3H), 1.61-1.74 (m, 4H), 2.24-2.33 (m, 2H), 2.60-2.67 (m, 2H), 2.75-2.78 (d, 2H), 3.17 (s, 2H), 3.20-3.24 (d, 3H), 3.32-3.35 (m, 2H), 6.99 (s, 1H), 7.10 (s, 1H), 7.18-7.20 (d, 1H), 7.35 (s, 1H), 7.49-7.53 (t, 1H), 7.63-7.69 (t, 1H), 7.83 (s, 1H), 8.39 (s, 1H).

### Example 112. Synthesis of Compound 112

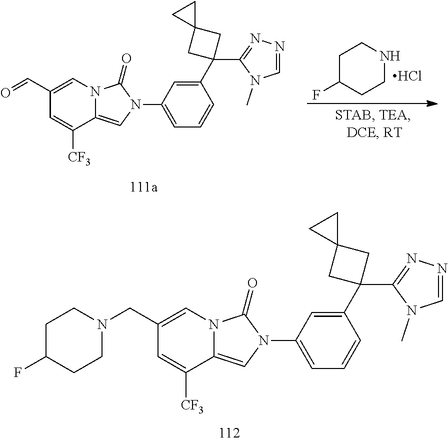 [SMILES: Cn1cnnc1C1(c2cccc(-n3cc4c(C(F)(F)F)cc(C=O)cn4c3=O)c2)CC2(CC2)C1 ; FC1CCNCC1 ; Cl ; Cn1cnnc1C1(c2cccc(-n3cc4c(C(F)(F)F)cc(CN5CCC(F)CC5)cn4c3=O)c2)CC2(CC2)C1]

### Synthesis of 112

To a stirred solution of 111a (250 mg, 0.535 mmol, 1.00 equiv) and 4-fluoropiperidine hydrochloride (223.98 mg, 1.605 mmol, 3 equiv) in DCE (5 mL) was added TEA (162.35 mg, 1.605 mmol, 3 equiv) at room temperature. The resulting mixture was stirred for 1h at room temperature. To the above mixture was added STAB (226.70 mg, 1.070 mmol, 2 equiv) at room temperature. The resulting mixture was stirred for additional 3h at room temperature. The reaction was quenched by the addition of saturated aqueous NH₄Cl (20 mL) at room temperature. The aqueous layer was extracted with CH₂Cl, (2×10 mL). The combined organic layers were concentrated under reduced pressure. The residue was purified by Prep-TLC (CH₂Cl₂/MeOH 20:1) to afford the crude product. The crude product (260 mg) was purified by Prep-HPLC with the following conditions (Column: XBridge Prep OED C18 Column, 30*150 mm, 5 μm; Mobile Phase A: Water (10 mmol/L NH₄HCO₃), Mobile Phase B: ACN; Flow rate: 60 mL/min; Gradient: 37% B to 57% B in 8 min, Wave Length: 220 nm; RT1 (min): 7.62) to afford Compound 112 (130.7 mg, 43.89%) as a yellow solid.

LC-MS: (ES, m/z): [M+H]⁺ 555

H-NMR: (400 MHz, CD₃OD, δ ppm): 0.53-0.57 (m, 2H), 0.63-0.67 (m, 2H), 1.84-2.05 (m, 4H), 2.47-2.53 (m, 2H), 2.65-2.73 (m, 2H), 2.81-2.90 (m, 2H), 3.22-3.29 (m, 2H), 3.32 (s, 3H), 3.33 (s, 2H), 4.59-4.80 (m, 1H), 7.11-7.15 (m, 2H), 7.30-7.33 (m, 1H), 7.54-7.62 (m, 2H), 7.68 (s, 1H), 7.84 (s, 1H), 8.41 (s, 1H).

### Example 113. Synthesis of Compound 113

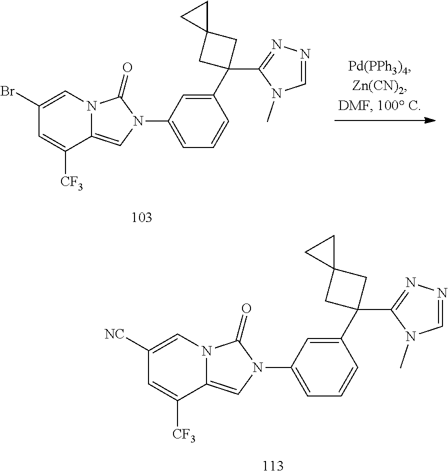 [SMILES: Cn1cnnc1C1(c2cccc(-n3cc4c(C(F)(F)F)cc(Br)cn4c3=O)c2)CC2(CC2)C1 ; Cn1cnnc1C1(c2cccc(-n3cc4c(C(F)(F)F)cc(C#N)cn4c3=O)c2)CC2(CC2)C1]

### Synthesis of 113

To a stirred mixture of 103 (600 mg, 1.158 mmol, 1.00 equiv) and Zn(CN)₂ (271.89 mg, 2.316 mmol, 2 equiv) in dioxane (6 mL) was added Pd(PPh₃)₄ (133.76 mg, 0.116 mmol, 0.1 equiv) at room temperature under nitrogen atmosphere. The resulting mixture was stirred for 4 h at 100 degrees C. under nitrogen atmosphere. The reaction was quenched by the addition of saturated aqueous NH₄Cl (30 mL). The aqueous layer was extracted with EtOAc (4×20 mL). The resulting mixture was concentrated under reduced pressure. The residue was purified by Prep-TLC (CH₂Cl₂/MeOH=20:1) to afford 113 (400 mg) as a yellow solid. The crude product (400 mg) was purified by Prep-HPLC with the following conditions (Column: SunFire C18 OBD Prep Column, 19*250 mm, 5 μm; Mobile Phase A: water (0.1% FA), Mobile Phase B: ACN; Flow rate: 30 mL/min; Gradient: 42% B to 52% B in 8 min, Wave Length: 254; 220 nm; RT1 (min): 7.53) to afford Compound 113 (143.0 mg, 26.6%) as a yellow solid,

LCMS: (ES, m/z): [M+H]⁺ 465.

H-NMR: (400 MHz, DMSO-d₆, ppm): δ 0.47-0.49 (m, 2H), 0.55-0.59 (m, 2H), 2.75-2.78 (d, 2H), 3.17-3.23 (m, 5H), 7.22-7.27 (m, 2H), 7.51-7.55 (m, 2H), 7.61-7.64 (d, 1H), 7.79 (s, 1H), 8.39 (s, 1H), 8.70 (s, 1H).

### Example 114. Synthesis of Compound 114

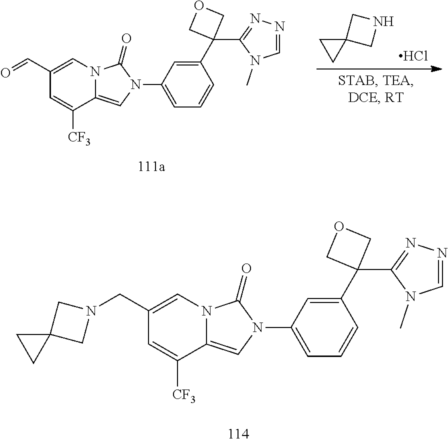 [SMILES: Cn1cnnc1C1(c2cccc(-n3cc4c(C(F)(F)F)cc(C=O)cn4c3=O)c2)COC1 ; C1CC12CNC2 ; Cl ; Cn1cnnc1C1(c2cccc(-n3cc4c(C(F)(F)F)cc(CN5CC6(CC6)C5)cn4c3=O)c2)COC1]

### Synthesis of 114

To a stirred mixture of 111a (400 mg, 0,856 mmol, 1.00 equiv) and 5-azaspiro[2.3]hexane hydrochloride (307.00 mg, 2.568 mmol, 3 equiv) in DCE (5 mL) were added Et₃N (259.77 mg, 2,568 mmol, 3 equiv) and STAB (362.72 ng, 1.712 mmol, 2 equiv) at room temperature. The resulting mixture was stirred overnight at room temperature. The reaction was quenched with sat NH₄Cl (aq.) (10 mL) at room temperature. The aqueous layer was extracted with EtOAc (3×10 mL). The resulting mixture was concentrated under reduced pressure. The residue was purified by Prep-TLC (CH₂Cl₂/MeOH 10:1) to afford 114 (150 mg, crude). The crude product (150 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 40% B to 56% B in 8 min; Wave Length: 254/220 nm; RT1 (min): 7.75) to afford Compound 114 (57.1 mg, 12.48%) as a yellow solid.

LC-MS: (ES, m/z): [M+H]⁺ 535

H-NMR: (400 MHz, DMSO, δ ppm): 0.45-0.50 (m, 6H), 0.55-0.59 (m, 2H), 2.75-2.78 (d, 2H), 3.17-3.24 (t, 2H), 3.28-3.32 (m, 3H), 3.33-3.44 (m, 4H), 3.45 (s, 2H), 7.00 (s, 1H), 7.17-7.19 (d, 1H), 7.34 (s, 1H), 7.48-7.52 (m, 1H), 7.53-7.56 (m, 1H), 7.63-7.68 (t, 1H), 7.84 (s, 1H), 8.39 (s, 1H).

### Example 115. Synthesis of Compound 115

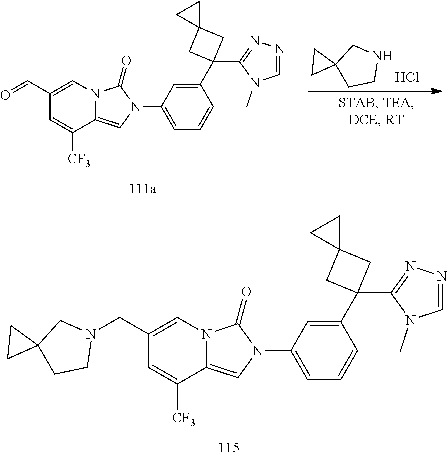

### Synthesis of 115

To a stirred mixture of 111a (600 mug, 1.284 mmol, 1.00 equiv) and 5-azaspiro[2.4]heptane hydrochloride (187.07 mg, 1.926 mmol, 1.5 equiv) in DCE (6 mL) was added TEA (259.77 mg, 2.568 mmol, 2 equiv) at room temperature. The resulting mixture was stirred for 30 min at room temperature. To the above mixture was added STAB (816.11 mg, 3.852 mmol, 3 equiv) at room temperature. The resulting mixture was stirred overnight at room temperature. The resulting mixture was diluted with water (10 mL). The resulting mixture was extracted with CH₂Cl₂ (3×20 mL). The combined organic layers were concentrated under reduced pressure. The residue was purified by Prep-TLC (CH₂Cl₂/MeOH=15:1) to afford 115 as a yellow solid. The crude product (120 mg) was purified by Prep-HPLC with the following conditions (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 40% B to 58% B in 8 min, Wave Length: 220 nm; RT1 (min): 7.7) to afford Compound 115 (95.2 mg, 13.52%) as a yellow solid.

LCMS: (ES, m/z): [M+H]⁺ 549.

H-NMR: (400 MHz, DMSO-d₆, ppm): δ 0.55-0.59 (m, 8H), 1.73-1.77 (m, 2H), 2.46 (s, 2H), 2.67-2.68 (m, 2H), 2.70-2.76 (m, 2H), 3.17-3.20 (m, 2H), 3.24 (s, 3H), 3.40 (s, 2H), 7.04 (s, 1H), 7.18-7.20 (m, 1H), 7.35 (s, 1H), 7.48-7.52 (m, 1H), 7.63-7.65 (m, 1H), 7.69 (s, 1H), 7.83 (s, 1H), 8.39 (s, 1H).

### Example 116. Synthesis of Compound 116

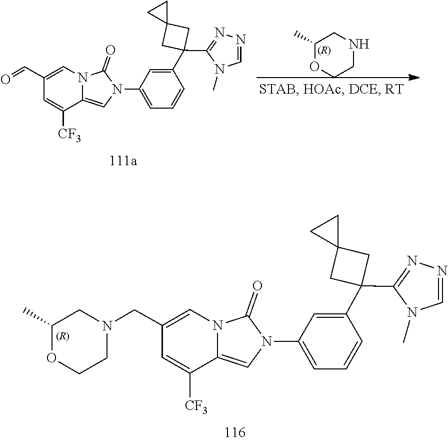

### Synthesis of 116

To a stirred solution of 111a (10 mg, 0,021 mmol, 1.00 equiv) and (2R)-2-methylmorpholine (3.25 mg, 0.032 mmol, 1.5 equiv) in DCE (9 mL) were added TEA (4.33 mg, 0.042 mmol, 2 equiv) and STAB (13.60 mg, 0.063 mmol, 3 equiv) at room temperature. The resulting mixture was stirred overnight at room temperature. The resulting mixture was diluted with water (15 mL). The resulting mixture was extracted with CH₂Cl₂ (3×20 mL), The combined organic layers were concentrated under reduced pressure. The residue was purified by Prep-TLC (CH₂Cl₂/MeOH=15:1) to afford 116 as a yellow solid. The crude product (400 mg) was purified by Prep-HPLC with the following conditions (Column: XBridge Prep OBD C18 Column, 30*150 mm, 5 μm; Mobile Phase A: water (10 mmol/L NH₄CO₃), Mobile Phase B: ACN; Plow rate: 60 mL/min; Gradient: 30% B to 55% B in 8 min, Wave Length: 220 nm; RT1 (min): 7.42) to afford Compound 116 (129.5 mg, 12.17%) as a yellow solid.

LCMS: (ES, m/z): [M+H]⁺ 553.

H-NMR: (400 MHz, DMSO-d₆, ppm): δ 0.45-0.49 (m, 2H), 0.55-0.57 (m, 2H), 1.03-1.05 (d, 3H), 1.74-1.74 (m, 1H), 2.04-2.08 (m, 1H), 2.53-2.68 (m, 1H), 2.72-2.78 (m, 3H), 3.17-3.24 (m, 2H), 3.27 (s, 3H), 3.28-3.32 (m, 2H), 3.48-3.51 (m, 2H), 3.72-3.75 (m, 1H), 7.01 (s, 1H), 7.18-7.20 (d, 1H), 7.35 (s, 1H), 7.49-7.53 (m, 1H), 7.63-7.65 (m, 1H), 7.65 (s, 1H), 7.83 (s, 1H), 8.39 (s, 1H).

### Example 117. Synthesis of Compound 117

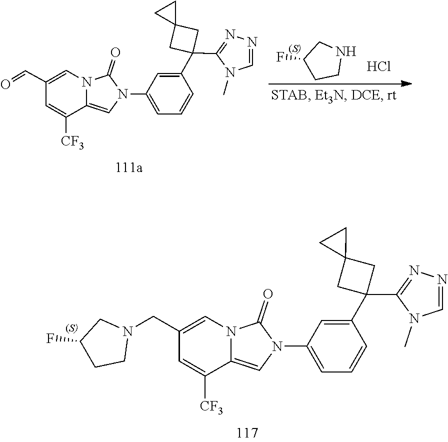

### Synthesis of 117

To a stirred mixture of 111a (450 mg, 0.963 mmol, 1.00 equiv) and (S)-3-fluoropyrrolidine hydrochloride (128.68 mg, 1.444 mmol, 1.5 equiv) in DCE (5 mL) was added Et₃N (291.78 mg 2,889 mmol, 3 equiv) at room temperature. The resulting mixture was stirred for 30 ruin at room temperature. To the above mixture was added STAB (612.08 mg 2.889 mmol, 3 equiv) at room temperature. The resulting mixture was stirred overnight at room temperature. The resulting mixture was diluted with water (15 mL). The resulting mixture was extracted with CH₂Cl₂ (3×20 mL). The combined organic layers were concentrated under reduced pressure. The residue was purified by Prep-TLC (CH₂Cl₂/MeOH 15:1) to afford 117 (150 mg) as a yellow solid. The crude product (150 mg) was purified by Prep-HPLC with the following conditions (Column: XBridge Prep OBD C18 Column, 30*150 mm, 5 μm; Mobile Phase A: water (10 mmol/L NH₄CO₃) Mobile Phase B: ACN; Flow rate: 60 mL/min; Gradient: 28% B to 50% B in 11 min, Wave Length: 220 nm; RT1 (min): 10.83) to afford Compound 117 (81.8 mg, 15.72%) as a yellow solid,

LCMS: (ES, m/z): [M+H]⁺ 541.

H-NMR: (400 MHz, DMSO-d₆, ppm): δ 0.45-0.49 (m, 2H), 0.55-0.59 (m, 2H), 1.78-2.00 (m, 1H), 2.01-2.25 (m, 1H), 2.34-2.36 (m, 1H), 2.59-2.71 (m, 1H), 2.75-2.83 (m, 4H), 3.17-3.20 (m, 2H), 3.28 (s, 3H), 3.43 (s, 2H), 5.14-5.28 (m, 1H), 7.03 (s, 1H), 7.18-7.20 (d, 1H), 7.36 (s, 1H), 7.49-7.53 (m, 1H), 7.63-7.66 (m, 1H), 7.72 (s, 1H), 7.84 (s, 1H), 8.39 (s, 1H).

### Example 118 Synthesis of Compound 11R

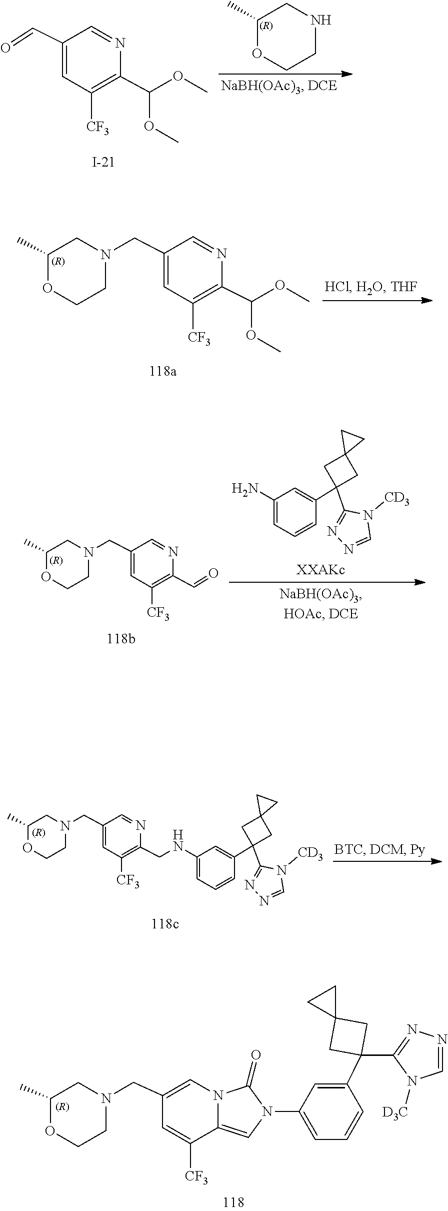

### Synthesis of 118a

Into a 25 mL vial were added I-2i (350 mg, 1.405 mmol, 1 equiv), DCE (5 mL), and (2R)-2-methylmorpholine (170.48 mg, 1.686 mmol, 1.2 equiv) at room temperature. To the above mixture was added STAB (595.37 mg, 2,810 mmol, 2 equiv) at room temperature. The resulting mixture was stirred overnight at room temperature. The reaction was quenched with saturated aqueous NH₄Cl (50 mL) at room temperature. The aqueous layer was extracted with EtOAc (3×20 mL) The resulting mixture was concentrated under vacuum. The residue was purified by Prep-TLC (PE/EA 2:1) to afford 118a (300 mg, 58.77%) as a brown oil.

### Synthesis of 118b

Into a 25 mL vial were added 118a (300 ng, 0.897 mmol, 1 equiv) and THF (2 mL) at room temperature. To the above mixture was added HC (5 mL, 1 mol/L) dropwise at room temperature. The resulting mixture was stirred for 2 h at 80° C. The residue was neutralized to pH 7 with saturated aqueous NaHCO₃. The aqueous layer was extracted with EtOAc (3×20 mL) The resulting mixture was concentrated under vacuum. The residue was purified by Prep-TLC (PE/EA 5:1) to afford 118b (220 mg, 79.10%) as a brown oil.

### Synthesis of 118c

To a stirred solution of 102c (200 mg, 0.777 mmol, 1 equiv) and 118b (224.03 mg, 0.777 mmol, 1 equiv) in DCE (5 mL) were added HOAc (46.67 mg, 0.777 mmol, 1 equiv) and NaBH(OAc)₃ (329.41 mg, 1.554 mmol, 2 equiv). The resulting mixture was stirred overnight at room temperature. The reaction was quenched by the addition of water (15 mL). The resulting mixture was extracted with CH₂Cl₂/MeOH=10/1 (2×20 mL). The combined organic layers were concentrated under reduced pressure. The residue was purified by Prep-TLC (CH₂Cl₂/MeOH 20:1) to afford 118c (270 mg, 65.60%) as a white solid.

### Synthesis of 118

To a stirred solution of 118c (260 mg, 0.491 mmol, 1 equiv) and Pyridine, 6 equiv) in DCM (10 mL) were added Triphosgene (58.27 mg, 0.196 mmol, 0.4 equiv) at 0° C. The resulting mixture was stirred for 1 h at 0° C. The reaction was quenched by the addition of sat. NH₄Cl (aq.) (15 mL). The resulting mixture was extracted with CH₂Cl₂/MeOH=10/1 (3×20 mL). The combined organic layers were concentrated under reduced pressure. The residue was purified by Prep-TLC (CH₂Cl₂/MeOH 15:1) to afford Compound 118 (162.9 mg, 59.72%) as a yellow solid,

LC-MS: (ES, m/z): [M+H]⁺ 577

H-NMR: (400 MHz, DMSO-d6, ppm): δ 0.49-0.59 (m, 4H), δ 1.03-1.05 (d, 3H), δ 1.72-1.77 (m, 1H), δ 2.06-2.12 (m, 1H), δ 2.65-2.78 (m, 4H), δ 3.17-3.20 (m, 2H), 3.20-3.28 (m, 2H), 3.46-3.56 (m, 2H), 3.73-3.76 (m, 1H), δ 7.02 (s, 1H), 37.19-7.20 (d, 1H), δ 7.36 (s, 1H), δ 7.49-7.53 (m, 1H), 37.63-7.65 (d, 1H), 37.70 (s, 1H), 37.83 (s, 1H), 38.40 (s, 1H).

### Example 119. Synthesis of Compound 119

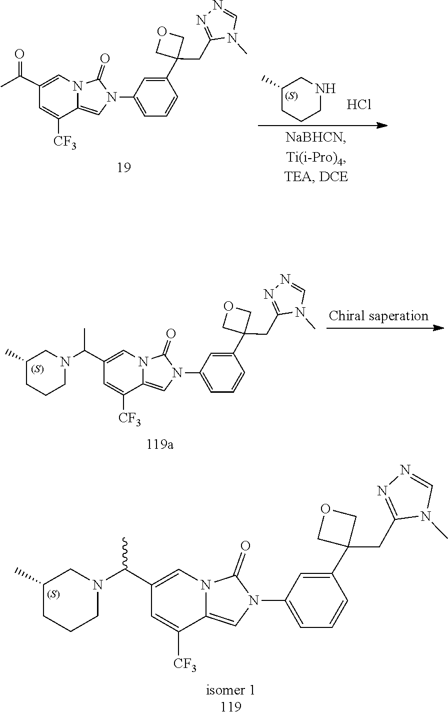

### Synthesis of 119a

To a stirred mixture of 19 (1 g, 2.121 mmol, 1 equiv) and (S)-3-METHYLPIPERIDINE HYDROCHLORIDE (1.44 g, 10,605 mmol, 5 equiv) TEA (2.15 g, 21.210 mmol, 10 equiv) in THF (30 mL) was added Ti(Oi-Pr)4 (3.01 g, 10.605 mmol, 5 equiv) at room temperature under nitrogen atmosphere. The resulting mixture was stirred for 6 h at 60° C., To the above mixture was added NaBH₃CN (0.27 g, 4,296 mmol, 2.03 equiv) at room temperature. The resulting mixture was stirred overnight at room temperature. The reaction was quenched by the addition of water (100 mL) at room temperature. The resulting mixture was extracted with EtOAc (3×50 mL). The combined organic layers were concentrated under reduced pressure. The residue was purified by reverse flash chromatography with the following conditions: column, silica gel; mobile phase, MeCN in water (10 mmoL/L NH₄HCO₃), 10% to 90% gradient in 30 min; detector, UV 254 nm. This resulted in 119a (350 mg, 29.75%) as a yellow solid.

### Synthesis of 119

119a (350 mg) was purified by chiral separation with the following conditions (Column: (R, R)-WHELK-O1-Kromasi, 5*25 cm, 5 μm; Mobile Phase A: MtBE (0.5% 2M NH₃-MeOH), Mobile Phase B: EtOH:DCM=1:1; Flow rate: 20 mL/min; Gradient: 30% B to 30% B in 15.5 min; Wave Length: 220/254 nm; RT1 (min): 10.51, RT2 (min): 12.60, the first peak is product) to afford Compound 119 (106.6 mg, 30.46%) as a yellow solid.

LC-MS: (ES, m/z): [M+H]⁺ 555

H-NMR: (400 MHz, DMSO-d6, ppm): δ 0.82-0.86 (m, 4H), δ 1.21-1.26 (d, 3H), δ 1.40-1.66 (m, 5H), δ 1.98-2.03 (m, 1H), δ 2.68-2.75 (m, 2H), δ 2.97 (s, 3H), δ 3.40-3.45 (m, 1H), δ 3.53 (s, 2H), δ 4.91-4.96 (m, 4H), δ 6.88-6.90 (d, 1H), δ 7.07 (s, 1H), δ 7.31 (s, 1H), δ 7.38-7.42 (m, 2H), δ 7.57 (s, 1H), δ 7.74-7.76 (m, 1H), δ 8.20 (s, 1H).

### Example 120. Synthesis of Compound 120

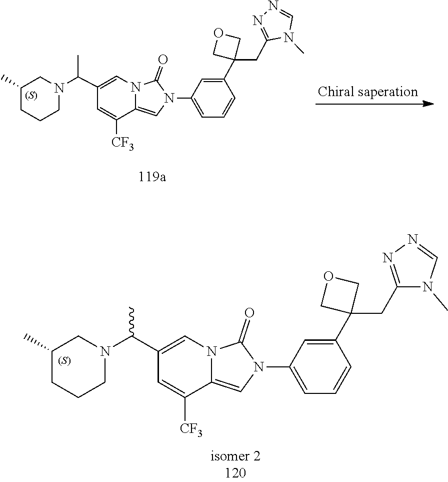

### Synthesis of 120

119a (300 mg, 0.541 mmol, 1.00 equiv) was purified by chiral separation with the following conditions (Column: (R, R)-WHELK-O1-Kromasi, 5*25 cm, 5 μm; Mobile Phase A: MtBE. (0.5% 2M NH₃-MeOH), Mobile Phase B: EtOH:DCM=1:1; Flow rate: 20 mL/min; Gradient: 30% B to 30% B in 15.5 min; Wave Length: 220/254 nm; RT1 (min): 10.51, RT2 (min): 12.60, the second peak is product) to afford Compound 120 (89.5 mg, 29.83%) as a yellow solid.

LC-MS: (ES, m/z): [M+H]⁺ 555

H-NMR: (400 MHz, DMSO-d6, ppm): δ 0.82-0.86 (m, 4H), δ 1.24 (s, 2H), δ 1.32-1.98 (m, 6H), δ 2.67-2.74 (m, 2H), δ 2.98 (s, 3H), δ 3.32-3.53 (m, 3H), δ 4.91-4.96 (m, 4H), 6.88-6.90 (d, 1H), δ 7.07 (s, 1H), δ 7.31-7.43 (m, 5H), δ 7.74-7.76 (m, 1H), δ 8.20 (s, 1H).

### Example 121. Synthesis of Compound 121

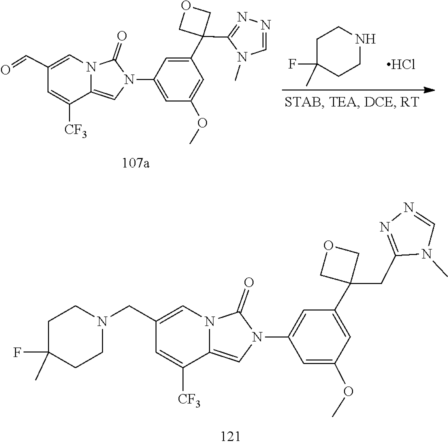

### Synthesis of 121

To a stirred mixture of 107a (190 mg, 0.401 mmol, 1 equiv) and 4-fluoro-4-methylpiperidine hydrochloride (184.97 mg, 1.203 mmol, 3 equiv) in DCE (2.00 mL) were added TEA (121.84 mg, 1.203 mmol, 3 equiv) and STAB (170.12 mg, 0.802 mmol, 2 equiv) at room temperature. The resulting mixture was stirred overnight at room temperature. The reaction was quenched with sat NH₄Cl (aq.) (10 mL) at room temperature. The aqueous layer was extracted with EtOAc (3×5 mL) The resulting mixture was concentrated under reduced pressure. The residue was purified by Prep-TLC (DCM/MeOH 20:1) to afford 121 (150 mg, crude). The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃). Mobile Phase B: ACN; Flow rate: 60 mL/min Gradient: 30% B to 51% B in 8 min; Wave Length: 220 nm; RT1 (min): 7.77 min) to afford Compound 121 (43.7 mg, 18.50%) as a yellow solid.

LC-MS: (ES, m/z): [M+H]⁺ 589

H-NMR: (400 MHz, DMSO, δ ppm): 1.71-1.74 (d, 3H), 1.75-1.79 (m, 4H), 2.24-2.33 (m, 2H), 2.58-2.76 (m, 2H), 3.00 (s, 3H), 3.32 (s, 2H), 3.52 (s, 2H), 3.71 (s, 3H), 4.89-4.94 (m, 4H), 6.40-6.41 (d, 1H), 7.00-7.01 (m, 2H), 7.33-7.37 (m, 2H), 7.76 (s, 1H), 8.22 (s, 1H).

### Example 122. Synthesis of Compound 122

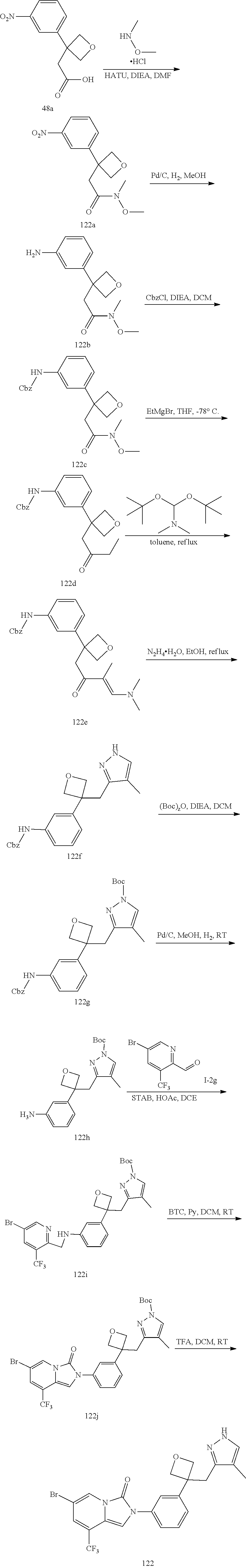

### Synthesis of 122a

To a stirred solution of 48a (10 g, 42.157 mmol, 100 equiv) and N,O-dimethylhydroxylamine (5.15 g, 84.314 mmol, 2 equiv) in DMF (150 mL) was added HATU (17.63 g, 46,373 mmol, 1.1 equiv) and DIEA (16.35 g, 126.471 mmol, 3 equiv) at room temperature. The resulting mixture was stirred for 3h at room temperature. The resulting mixture was diluted with water (300 mL). The precipitated solids were collected by filtration and washed with water (1×20 mL). The resulting mixture was concentrated under vacuum. This resulted in 122a (11 g, 83.79%) as an off-white solid.

### Synthesis of 122b

A solution of 122a (10 g, 35,679 mmol, 1.00 equiv) and Pd/C (0.99 g, 9.277 mmol, 0.26 equiv) in MeOH (300 mL, 0.046 mmol) was stirred for 6 h at room temperature under hydrogen atmosphere. The resulting mixture was filtered through a celite pad and the filter cake was washed with MeOH (2×50 mL). The filtrate was concentrated under reduced pressure. This resulted in 122b (9 g, 90.70%) as a light yellow oil,

### Synthesis of 122c

To a stirred solution of 122b (10 g, 39.952 mmol, 1.00 equiv) and DIEA (15.49 g, 119.856 mmol, 3 equiv) in DCM (200 mL) was added CbzCl (13.63 g, 79.904 mmol, 2 equiv) dropwise at room temperature under nitrogen atmosphere. The resulting mixture was stirred overnight at room temperature under nitrogen atmosphere. The resulting mixture was washed with 100 mL of water. The organic layer was concentrated under reduced pressure. The residue was purified by silica gel column chromatography, eluted with PE/EA (10:1) to afford 122c as an off-white solid.

### Synthesis of 122d

To a stirred solution of 122c (8 g, 20.810 mmol, 1.00 equiv) in THF (100 mL) was added EtMgBr (62.4 ml, 62,430 mmol, 3 equiv, 1M) dropwise at −78 degrees C. under nitrogen atmosphere. The resulting mixture was stirred for 4h at room temperature under nitrogen atmosphere. The reaction was quenched by the addition of saturated aqueous NH₄Cl (100 mL) at room temperature. The aqueous layer was extracted with EtOAc (2×100 mL), The residue was purified by silica gel column chromatography, eluted with PE/EA (15:1) to afford 122d as an off-white solid.

### Synthesis of 122e

To a stirred solution of 122d (4 g, 11.318 mmol, 1.00 equiv) in methylbenzene (50 mL) was added [bis(tert-butoxy)methyl]dimethylamine (6.90 g, 33.954 mmol, 3 equiv) at room temperature. The resulting mixture was stirred overnight at 100 degrees C. The resulting mixture was concentrated under vacuum. This resulted in 122e (5 g, 91.92%) as a yellow oil. The crude product was used in the next step directly without further purification.

### Synthesis of 122f

To a stirred solution of 122e (5 g, 12.240 mmol, 1.00 equiv) in EtOH (2 mL) was added hydrazine hydrate (98%) (61.27 mg, 1.220 mmol, 10 equiv) at room temperature under nitrogen atmosphere. The resulting mixture was stirred overnight at 80 degrees C. The resulting mixture was concentrated under vacuum. The residue was purified by silica gel column chromatography, eluted with DCM/MeOH-1 (30:1) to afford 122f (3 g, 55.20%) as a light yellow oil.

### Synthesis of 122g

To a stirred solution of 122f (3 g, 7.948 mmol, 1.00 equiv) and (Boc)₂O (5.20 g, 23.844 mmol, 3 equiv) in DCM (150 mL) was added DIEA (4.11 g, 31,792 mmol, 4 equiv) at room temperature under nitrogen atmosphere. The resulting mixture was stirred overnight at room temperature. The resulting mixture was washed with 100 mL of water. The residue was purified by silica gel column chromatography, elated with CH₂Cl₂/MeOH (50:1) to afford 122g (1.3 g, 30.82%) as an off-white solid.

### Synthesis of 122h

To a solution of 122g (13 g, 2.780 mmol, 1.00 equiv) in MeOH (30 mL) was added Pd/C (100 mg, 0.940 mmol, 0.34 equiv) under nitrogen atmosphere in a 100 mL round-bottom flask. The mixture was hydrogenated at room temperature overnight under hydrogen atmosphere using a hydrogen balloon, filtered through a Celite pad, and concentrated under reduced pressure. This resulted in 122h (850 mg, 80.12%) as an off-white solid.

### Synthesis of 122i

To a stirred solution of 122h (350 mg, 1.019 mmol, 1.00 equiv) and I-2g (258.87 mg, 1.019 mmol, 1 equiv) in DCE (10 mL) was added HOAc (61.20 mg, 1.019 mmol, 1 equiv) and STAB (431.99 mg, 2.038 mmol, 2 equiv) at room temperature under nitrogen atmosphere. The resulting mixture was stirred for 2h at room temperature. The reaction was quenched by the addition of NH₄Cl (aq.) (20 mL) at room temperature. The aqueous layer was extracted with DCM (2×20 mL). The organic layers were concentrated under reduced pressure. The residue was purified by Prep-TLC (CH₂Cl₂/MeOH 20:1) to afford 122i (400 mg, 62.78%) as an off-white solid.

### Synthesis of 122j

To a stirred solution of 122i (380 mg, 0.654 mmol, 1.00 equiv) and Pyridine (516.96 mg, 6.540 mmol, 10 equiv) in DCM (15 mL) was added BTC (77.43 mg, 6.540 mmol, 0.4 equiv) at room temperature. The resulting mixture was stirred for 10 min at room temperature. The reaction was quenched with saturated aqueous NaHCO₃ at room temperature. The aqueous layer was extracted with DCM (2×10 mL). The residue was purified by Prep-TLC (CH₂Cl₂/MeOH 30:1) to afford 122j (310 mg, 72.62%) as a yellow solid,

### Synthesis of 122

To a stirred solution of 122j (280 mg, 0.461 mmol, 1.00 equiv) in DCM (5 mL) was added TFA (1 mL) at room temperature. The resulting mixture was stirred for 3h at room temperature. The resulting mixture was concentrated under vacuum. The residue was purified by reverse flash chromatography with the following conditions: column, C18 silica gel; mobile phase, MeCN in water, 0% to 55% gradient in 15 min; detector, UV 254 nm. This resulted in Compound 122 (133.0 mg, 54.14%) as a yellow solid.

LC-MS: (ES, m/z): [M+H]⁺ 507

H-NMR: (400 MHz, CD₃OD, δ ppm): 0.98 (s, 3H), 3.37 (s, 2H), 5.02 (s, 4H), 6.90-6.92 (m, 1H), 7.05-7.10 (m, 3H), 7.20 (s, 1H), 7.42-7.46 (m, 1H), 7.58-7.61 (d, 1H), 7.99 (s, 1H).

### Example 123. Synthesis of Compound 123

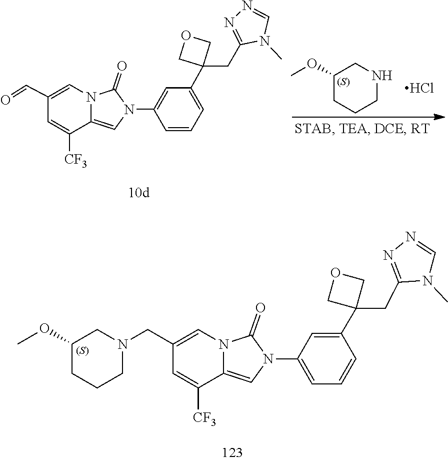

### Synthesis of 123

To a stirred solution of 10d (150 mg, 0.328 mmol, 1.00 equiv) and (S)-3-methoxypiperidine hydrochloride (149.17 mg, 0.984 mmol, 3 equiv) in DCE (5 mL) was added TEA (99.55 mg, 0.984 mmol, 3 equiv) at room temperature under nitrogen atmosphere. The resulting mixture was stirred for 1h at room temperature. To the above mixture was added STAB (139.00 mg, 0.656 mmol, 2 equiv) at room temperature. The resulting mixture was stirred for an additional 5h at room temperature. The reaction was quenched by the addition of saturated aqueous NH₄Cl (20 mL) at room temperature. The aqueous layer was extracted with DCM (2×20 mL). The organic layers were concentrated under reduced pressure. The residue was purified by reverse flash chromatography with the following conditions: column, C18 silica gel; mobile phase A: water (10 mmol/L), mobile phase B: MeCN, 0% B to 40% B gradient in 15 min; detector, UV 254 nm. This resulted in Compound 123 (45.0 mg, 24.65%) as a yellow solid.

LC-MS: (ES, m/z): [M+H]⁺ 557

H-NMR: (400 MHz, CD₃OD, δ ppm): 1.25-1.37 (m, 1H), 1.37-1.57 (m, 1H), 1.74-1.85 (m, 1H), 1.93-1.99 (m, 1H), 2.17-2.23 (m, 2H), 2.64-2.66 (m, 1H), 2.86-2.88 (m, 1H), 2.96 (s, 3H), 3.33-3.41 (m, 6H), 3.68 (s, 2H), 5.10 (s, 4H), 6.88-6.91 (m, 1H), 7.11-7.14 (m, 2H), 7.31-7.32 (d, 1H), 7.44-7.48 (m, 1H), 7.62-7.65 (m, 1H), 7.68 (s, 1H), 8.20 (s, 1H).

### Example 124. Synthesis of Compound 124

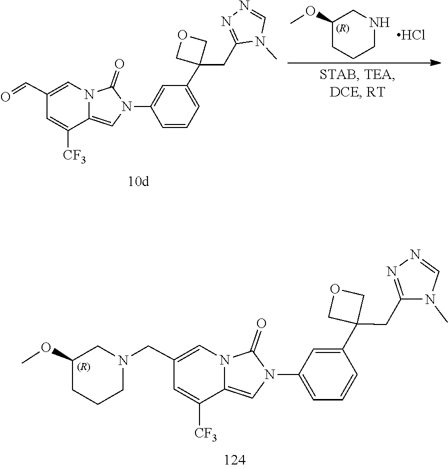

### Synthesis of 124

To a stirred solution of 10d (150 mg, 0,328 mmol, 1.00 equiv) and (R)-3-methoxypiperidine hydrochloride (149.17 mg, 0.984 mmol, 3 equiv) in DCE (5 mL) were added TEA (99.55 mg, 0.984 mmol, 3 equiv) at room temperature under nitrogen atmosphere. The resulting mixture was stirred for 1h at room temperature. To the above mixture was added STAB (139.00 mg, 0.656 mmol, 2 equiv) at room temperature. The resulting mixture was stirred for additional 5h at room temperature. The reaction was quenched by the addition of saturated aqueous NH₄Cl (20 mL) at room temperature. The aqueous layer was extracted with DCM (2×20 mL). The organic layers were concentrated under reduced pressure. The residue was purified by reverse flash chromatography with the following conditions: column, C18 silica gel; mobile phase A: water (10 mmol/L), mobile phase B: MeCN, 0% B to 40% B gradient in 15 min; detector, L 254 nm. This resulted in 124 (39.8 mg, 21.81%) as a yellow solid.

LC-MS: (ES, m/z): [M+H]⁺ 557

H-NMR: (400 MHz, CD₃OD, δ ppm): 1.25-1.37 (m, 1H), 1.37-1.57 (m, 1H), 1.74-1.85 (m, 1H), 1.93-1.99 (m, 1H), 2.17-2.23 (m, 2H), 2.64-2.66 (m, 1H), 2.86-2.88 (m, 1H), 2.96 (s, 3H), 3.33-3.41 (m, 6H), 3.68 (s, 2H), 5.10 (s, 4H), 6.89-6.91 (m, 1H), 7.11-7.14 (m, 2H), 7.31-7.32 (d, 1H), 7.44-7.48 (m, 1H), 7.62-7.65 (m, 1H), 7.68 (s, 1H), 8.20 (s, 1H).

### Example 125. Synthesis of Compound 125

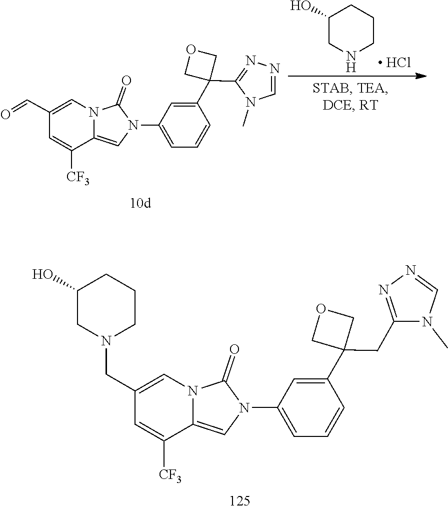 [SMILES: Cn1cnnc1C1(c2cccc(-n3cc4c(C(F)(F)F)cc(C=O)cn4c3=O)c2)COC1 ; O[C@@H]1CCCNC1 ; "· HCl" ; Cn1cnnc1CC1(c2cccc(-n3cc4c(C(F)(F)F)cc(CN5CCC[C@@H](O)C5)cn4c3=O)c2)COC1]

### Synthesis of 125

To a stirred mixture of 10d (200 mg, 0.422 mmol, 1 equiv) and (3R)-piperidin-3-ol hydrochloride (174.41 mg, 1.266 mmol, 3 equiv) in DCE (3.00 mL) were added TEA (128.25 mg, 1.266 mmol, 3 equiv) and STAB (179.08 mg, 0.844 mmol, 2 equiv) at room temperature. The resulting mixture was stirred overnight at room temperature. The reaction was quenched with sat. NH₄Cl (aq.) (10 mL) at room temperature. The aqueous layer was extracted with EtOAc (3×5 mL). The resulting mixture was concentrated under reduced pressure. The residue was purified by Prep-TLC (DCM/MeOH 20:1) to afford 125 (150 mg, crude). The crude product (150 mg) was purified by Prep-HPLC with the following conditions (Column: YMC-Actus Triart C18 ExRS, 30*150 mm, 5 μm; Mobile Phase A: Water (10 mmol/NH₄HCO₃+0.1% NH₃·H₂O), Mobile Phase B: ACN; Flow rate: 60 mL/min; Gradient: 18% B to 38% B in 8 min; Wave Length: 220 nm; RT1 (min): 7.95) to afford Compound 125 (79.9 mg, 34.86%) as yellow solid.

LC-MS: (ES, m/z): [M+H]⁺ 543

H-NMR: (400 MHz, DMSO, δ ppm): 0.98-1.19 (m, 1H), 1.35-1.49 (m, 1H), 1.66-1.71 (m, 1H), 1.72-1.82 (m, 2H), 1.87-1.94 (m, 1H), 2.65-2.68 (m, 1H), 2.80-2.82 (d, 1H), 2.97 (s, 3H), 3.23-3.26 (d, 2H), 3.45-3.46 (d, 1H), 3.53 (s, 2H), 4.60-4.61 (d, 1H), 4.91-4.96 (m, 4H), 6.88-6.90 (d, 1H), 7.01 (s, 1H), 7.31 (s, 1H), 7.38-7.42 (m, 2H), 7.67 (s, 1H), 7.74-7.76 (d, 1H), 8.20 (s, 1H).

### Example 126. Synthesis of Compound 126

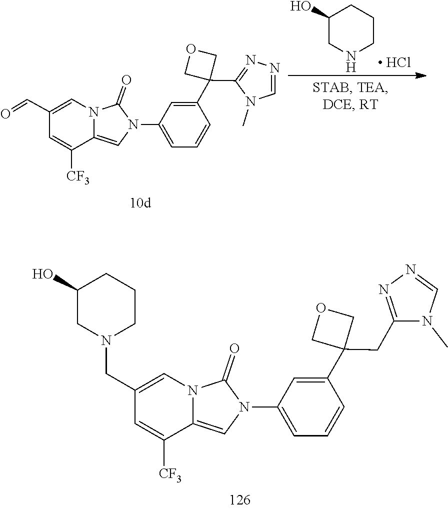 [SMILES: Cn1cnnc1C1(c2cccc(-n3cc4c(C(F)(F)F)cc(C=O)cn4c3=O)c2)COC1 ; O[C@H]1CCCNC1 ; "· HCl" ; Cn1cnnc1CC1(c2cccc(-n3cc4c(C(F)(F)F)cc(CN5CCC[C@H](O)C5)cn4c3=O)c2)COC1]

### Synthesis of 126

To a stirred mixture of 10d (200 mg, 0.422 mmol, 1 equiv) and (3S)-piperidin-3-ol hydrochloride (174.41 mg, 1.266 mmol, 3 equiv) in DCE (3.00 mL) were added TEA (128.25 mg, 1.266 mmol, 3 equiv) and NaBH(OAC)₃ (179.08 mg, 0.844 mmol, 2 equiv) at room temperature. The resulting mixture was stirred overnight at room temperature. The reaction was quenched with sat. NH₄Cl (aq.) (10 mL) at room temperature. The aqueous layer was extracted with EtOAc (3×5 ml). The resulting mixture was concentrated under reduced pressure. The residue was purified by Prep-TLC (DCM/MeOH 20:1) to afford 126 (150 mg, crude). The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 15% B to 37% B in 8 min; Wave Length: 220 nm; RT1 (min): 8.03) to afford Compound 126 (80.6 mg, 35.16%) as yellow solid,

LC-MS: (ES, m/z): [M+H]⁺ 543

H-NMR: (400 MHz, DMSO, δ ppm): 0.98-1.19 (m, 1H), 1.35-1.49 (m, 1H), 1.66-1.71 (m, 1H), 1.72-1.82 (m, 2H), 1.87-1.94 (m, 1H), 2.65-2.67 (m, 1H), 2.80-2.82 (d, 1H), 2.97 (s, 3H), 3.23-3.26 (d, 2H), 3.42-3.46 (d, 1H), 3.53 (s, 2H), 4.60-4.61 (d, 1H), 4.91-4.96 (m, 4H), 6.88-6.90 (d, 1H), 7.01 (s, 1H), 7.31 (s, 1H), 7.38-7.42 (m, 2H), 7.67 (s, 1H), 7.74-7.76 (d, 1H), 8.20 (s, 1H).

### Example 127. Synthesis of Compound 127

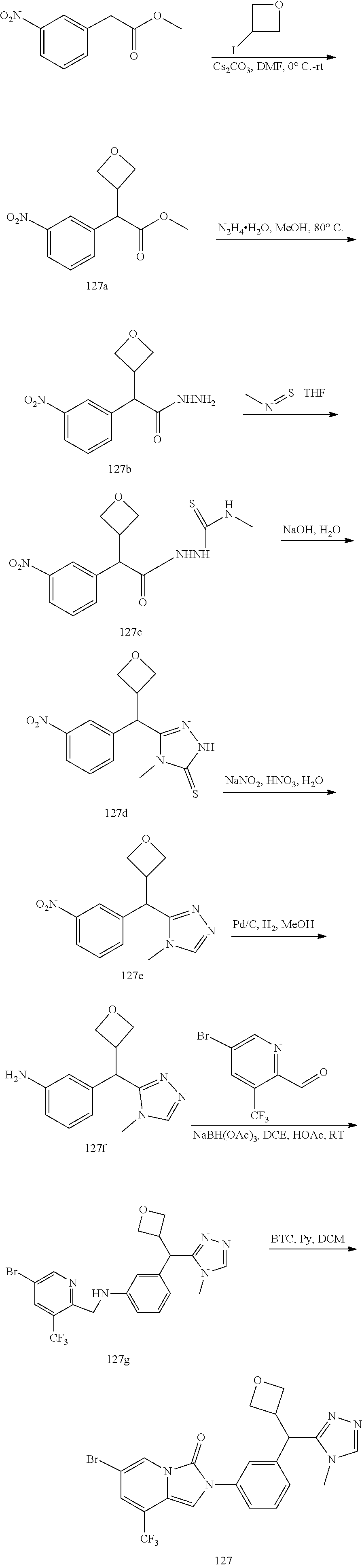

### Synthesis of 127a

To a stirred solution of methyl 2-(3-nitrophenyl)acetate (10 g, 51.236 mmol, 1 equiv) and 3-iodooxetane (18.85 g, 102.472 mmol, 2 equiv) in DMF (200 mL) was added Cs₂CO₃ (33.39 g, 102,472 mmol, 2 equiv) at 0° C. under nitrogen atmosphere. The resulting mixture was stirred overnight at room temperature under nitrogen atmosphere. The reaction was quenched with saturated aqueous NH₄Cl at room temperature. The resulting mixture was extracted with EtOAc (3×300 mL). The combined organic layers were washed with water (3×100 mL) and dried over anhydrous Na₂SO₄. After filtration, the filtrate was concentrated under reduced pressure. The residue was purified by silica gel column chromatography, eluted with PE/EA (10:1) to afford 127a (5 g, 35.35%) as a colorless oil.

### Synthesis of 127b

A solution of 127a (5 g, 19.901 mmol, 1 equiv) and NH₂NH₂·H₂O (7.97 g, 159.208 mmol, 8 equiv) in MeOH (50 mL) was stirred overnight at 90° C. under nitrogen atmosphere. The reaction was quenched with water at room temperature. The resulting mixture was extracted with EtOAc (3×100 mL). The combined organic layers were dried over anhydrous Na₂SO₄, After filtration, the filtrate was concentrated under reduced pressure. The residue was purified by silica gel column chromatography, eluted with PE/EA (5:1) to afford 127b (1.8 g, 35.28%) as a colorless oil.

### Synthesis of 127c

A solution of 127b (1.8 g, 7.164 mmol, 1 equiv) and methyl isothiocyanate (1.05 g, 14.328 mmol, 2 equiv) in THF (20 mL) was stirred overnight at room temperature under nitrogen atmosphere. The reaction was quenched by the addition of water (100 mL) at room temperature. The resulting mixture was concentrated under reduced pressure. The precipitated solids were collected by filtration and washed with water (3×10 mL) to afford 127c (2.2 g, 85.20%) as a white solid.

### Synthesis of 127d

A solution of 127c (2.15 g, 6,628 mmol, 1 equiv) and NaOH (2.12 g, 53,024 mmol, 8 equiv) in water (20 ml) was stirred overnight at room temperature under nitrogen atmosphere. The resulting mixture was diluted with water (50 mL). The mixture was acidified to pH 5 with conc. HCl. The precipitated solids were collected by filtration and washed with water (3×10 mL) to afford 127d (2 g, 91.60%) as a white solid,

### Synthesis of 127e

To a stirred solution of 127d (2 g, 6.529 mmol, 1 equiv) and NaNO₂ (4.50 g, 65.290 mmol, 10 equiv) in water (20 mL) was added HNO₃ (4.11 g, 65.290 mmol, 10 equiv) dropwise at 0° C. under nitrogen atmosphere. The resulting mixture was stirred for 2 h at room temperature under nitrogen atmosphere. The reaction was quenched with saturated aqueous NaHCO₃ at room temperature. The resulting mixture was extracted with EtOAc (3×100 ml). The combined organic layers were dried over anhydrous Na₂SO₄. After filtration, the filtrate was concentrated under reduced pressure to afford 127e (1.4 g, 71.93%) as a colorless oil.

### Synthesis of 127f

To a solution of 127e (1.4 g, 5.104 mmol, 1 equiv) in 30 mL MeOH was added Pd/C (0.42 g, 10%) under nitrogen atmosphere. The mixture was hydrogenated at room temperature overnight under hydrogen atmosphere using a hydrogen balloon, filtered through a Celite pad, and concentrated under reduced pressure. This resulted in 127f (1.3 g, 96.96%) as a yellow solid.

### Synthesis of 127g

To a stirred mixture of 127f (1 g, 4.093 mmol, 1 equiv) and 1-2g (1.56 g, 6,139 mmol, 1.5 equiv) in DCE (10 mL) were added NaBH(OAc)3 (2.60 g, 12.279 mmol, 3 equiv) and HOAc (0.25 g, 4.093 mmol, 1 equiv) at room temperature. The resulting mixture was stirred overnight at room temperature. The reaction was quenched by the addition of saturated aqueous NH₄Cl (10 mL) at room temperature. The resulting mixture was diluted with water (10 mL). The aqueous layer was extracted with CH₂Cl₂ (3×10 mL). The resulting mixture was concentrated under reduced pressure. The residue was purified by silica gel column chromatography, eluted with CH₂Cl₂/MeOH (50:1) to afford 127g (1.1 g, 50.70%) as a yellow solid.

### Synthesis of 127

Into a 50 ml 3-necked round-bottom flask were added 127g (1.1 g, 2.281 mmol, 1 equiv), DCM (15 mL), and Pyridine (1.08 g, 13,686 mmol, 6 equiv) at room temperature. To the above mixture was added bis(trichloromethyl) carbonate (0.24 g, 0.798 mmol, 0.35 equiv) at 0° C. The resulting mixture was stirred for additional 1 h at 0° C. The reaction was quenched with water (30 ml) at room temperature. The aqueous layer was extracted with DCM (3×10 mL). The resulting mixture was concentrated under vacuum. The residue was purified by silica gel column chromatography, eluted with CH₂Cl₂/MeOH (80:1) to afford Compound 127 (900 mg, 75.31%) as a yellow solid, 9.1 mg submitted,

LC-MS: (ES, m/z): [M+H]⁺ 508

H-NMR: (400 MHz, DMSO-d₆, ppm, δ): 3.37 (s, 3H), 3.87-3.97 (m, 1H), 4.26-4.33 (m, 1H), 4.47-4.49 (d, 2H), 4.78-4.81 (m, 2H), 7.17-7.21 (m, 2H), 7.48-7.50 (m, 2H), 7.74-7.75 (m, 2H), 8.03 (s, 1H), 8.37 (s, 1H).

### Example 128. Synthesis of Compound 128

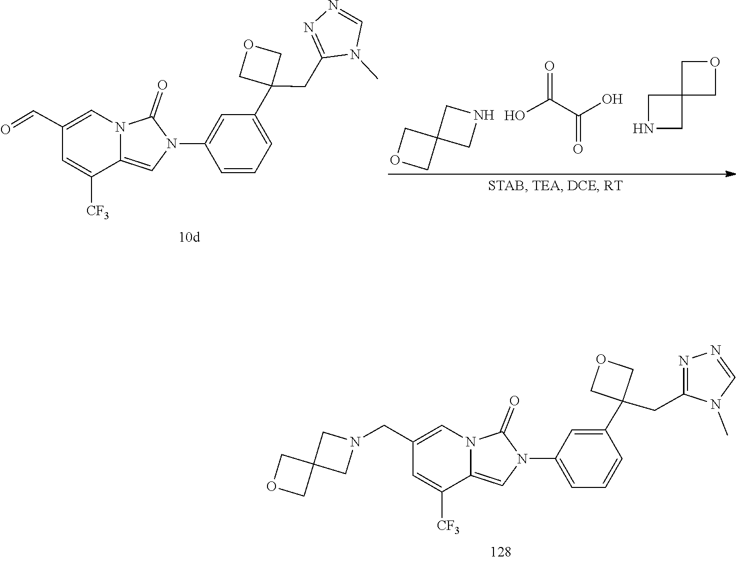

### Synthesis of 128

To a stirred solution of 10d (80 mg, 0.175 mmol, 1 equiv) and 2-oxa-6-azaspiro [3,3]heptane hemioxalate (60.49 mg, 0.210 mmol, 1.2 equiv) in DCE (2 mL) were added TEA (35.40 mg, 0.350 mmol, 2 equiv) and STAB (74.14 mg, 0.350 mmol, 2 equiv) at room temperature. The resulting mixture was stirred overnight at room temperature. The reaction was quenched by the addition of sat. NH₄Cl (aq.) (5 ml) at room temperature. The resulting mixture was extracted with EtOAc (3×8 mL) and the organic layers were dried over anhydrous Na₂SO₄. After filtration, the filtrate was concentrated under reduced pressure. The crude product was purified by reverse phase flash with the following conditions (Column: C18 silicagel column 40 g; Mobile Phase A: Water (0.1% NH₄CO₃), Mobile Phase B: ACN; Flow rate: 80 mL/min; Gradient: 30% B to 60% B in 15 min; 220 nm) to afford Compound 128 (47.7 mg, 50.45%) as a yellow solid.

LC-MS: (ES, m/z): [M+H]⁺:541

H-NMR: (400 MHz, DMSO-d6, ppm): δ 2.49-2.51 (m, 2H), δ 2.97 (s, 3H), 33.32-3.38 (m, 4H), δ 3.53 (s, 2H), δ 4.61 (s, 4H), δ 4.90-4.96 (m, 4H), δ 6.88-6.94 (m, 1H), δ 6.95-7.00 (m, 1H), δ 7.30 (s, 1H), δ 7.38-7.42 (m, 2H), δ 7.63 (s, 1H), δ 7.73-7.76 (m, 1H), δ 8.20 (s, 1H).

### Example 129. Synthesis of Compound 129

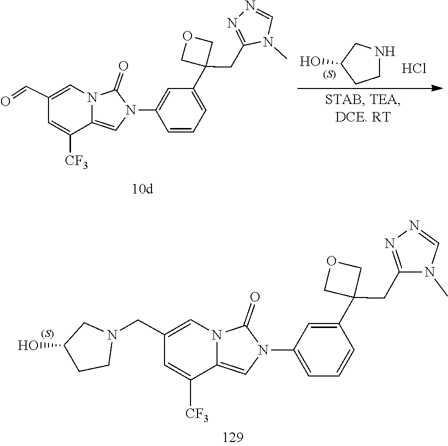

### Synthesis of 129

To a stirred solution of 10d (150 mg, 0.328 mmol, 1 equiv) and (3S)-pyrrolidin-3-ol hydrochloride (121.58 mg, 0.984 mmol, 3 equiv) in DCE (2 mL, 25,263 mmol) was added TEA (132 mg, 4 equiv) and STAB (139.00 mg, 0.656 mmol, 2 equiv) at room temperature. The reaction was quenched by the addition of water (20 ml). The resulting mixture was extracted with DCM (3×25 ml). The organic layers were concentrated under reduced pressure. The crude product (130 mg) was purified by Prep-HPLC with the following conditions (Column: XBridge Prep OBD C18 Column, 3*150 mm, 5 μm; Mobile Phase A: Water (10 mmol/L NH₄HCO₃), Mobile Phase B: ACN; Flow rate: 60 mL/min; Gradient: 10% B to 40% B in 8 min, Wave Length: 220 nm; RT1 (min): 7.57) to afford Compound 129 (47.3 mg 26.42%) as a yellow solid.

LCMS: (ES, m/z): [M+H]⁺ 529

H-NMR: H NMR (400 MHz, DMSO, ppm, δ): 1.43-1.65 (m, 1H), 1.91-2.09 (m, 1H), 2.31-2.38 (m, 1H), 2.38-2.47 (m, 1H), 2.58-2.65 (m, 1H), 2.68-2.72 (m, 1H), 2.97 (s, 3H), 3.39-3.45 (m, 2H), 3.54 (s, 2H), 4.20-4.21 (m, 1H), 4.71 (s, 1H), 4.90-5.02 (m, 4H), 6.76-6.95 (d, 1H), 7.02 (s, 1H), 7.31 (s, 1H), 7.38-7.42 (m, 2H), 7.68 (s, 1H), 7.74-7.77 (d, 1H), 8.20 (s, 1H).

### Example 130. Synthesis of Compound 130

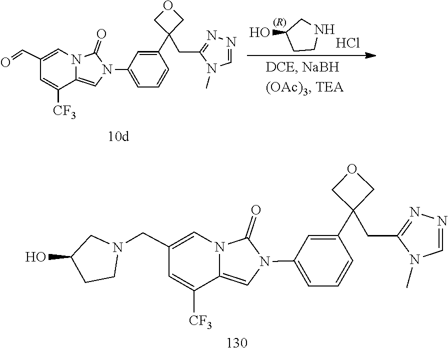

### Synthesis of 130

To a stirred solution of 10d (150 mg, 0.328 mmol, 1 equiv) and (3R)-pyrrolidin-3-ol hydrochloride (121.58 mg, 0.984 mmol, 3 equiv) in DCE (2 mL, 25.263 mmol) and TEA (132 mg, 4 equiv) was added STAB (139.00 mg, 0.656 mmol, 2 equiv) at room temperature. The resulting mixture was stirred overnight at room temperature. The reaction was quenched by the addition of water (10 ml). The resulting mixture was extracted with DCM/MeOH (5:1, 3×25 ml). The organic layers were concentrated under reduced pressure. The crude product (130 mg) was purified by Prep-HPLC with the following condition: Column: XBridge Prep OBD C18 Column, 30*150 mm, 5 μm; Mobile Phase A: water (10 mmol/L NH₄HCO₃), Mobile Phase B: ACN; Flow rate: 30 mL/min; Gradient: 11% B to 30% B in 11 min, 30% B; Wave Length: 220 nm; RT1 (min): 11.25 to afford 130 (48 mg 26.81%) as a yellow solid.

LCMS: (ES, m/z): [M+H]⁺ 529

H-NMR: H NMR (400 MHz, DMSO, ppm): 51.49-1.54 (d, 1H), δ 1.93-2.13 (m, 1H), δ 2.23-2.40 (t, 1H), δ 2.40-2.48 (m, 1H), δ 2.57-2.66 (d, 1H), δ 2.66-2.72 (m, 1H), δ 2.91-3.03 (d, 3H), δ 3.37-3.49 (t, 2H), δ 3.49-3.61 (m, 2H), δ 4.08-4.29 (m, 1H), δ 4.62-4.81 (d, 1H), δ 4.85-5.05 (m, 4H), δ 6.88-6.90 (d, 1H), δ 7.03 (s, 1H), δ 7.31 (s, 1H), δ 7.38-7.42 (m, 2H), δ 7.68 (s, 1H), δ 7.75-7.77 (m, 1H), δ 8.20 (s, 1H).

### Example 131. Synthesis of Compound 131

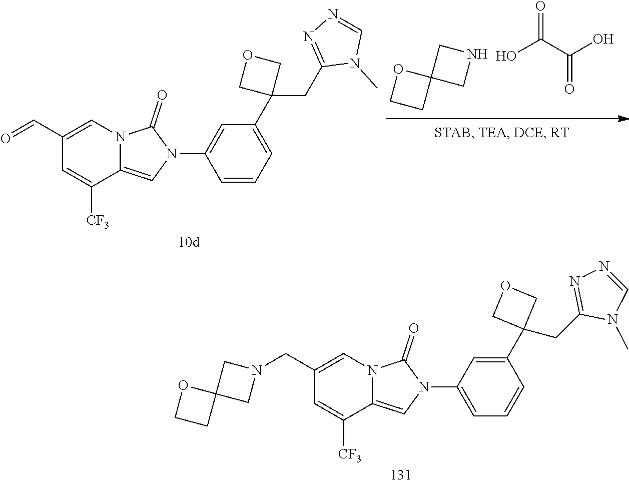

### Synthesis of 131

To a stirred solution of 10d (80 mg, 0.175 mmol, 1 equiv) and 1-Oxa-6-azaspiro[3.3]heptane oxalate (2:1) (50.4 mg, 0.350 mmol, 1 equiv) in DCE (1.5 mL) were added TEA (35.40 mg, 0.350 mmol, 2 equiv) and STAB (74.14 mg, 0.350 mmol, 2 equiv) at room temperature under nitrogen atmosphere. The resulting mixture was stirred overnight at room temperature. The reaction was quenched by water (5 mL) at room temperature. The resulting mixture was extracted with DCM (3×8 mL). The organic layers were concentrated under reduced pressure. The residue was purified by reverse flash chromatography with the following conditions: column, C18 silica gel; mobile phase, Mobile Phase A: Water (10 mmol/L NH₄HCO₃), Mobile Phase B: CAN, 55% B to 60% B gradient in 10 min; detector, UV 254 nm. This resulted in Compound 131 (54.4 mg, 56.62%) as a yellow solid.

LC-MS: (ES, m/z): [M+H]⁺:541

H-NMR: (400 MHz, DMSO-d6, ppm): δ 2.74-2.78 (m, 2H), δ 2.97 (s, 3H), δ 3.11-3.13 (d, 2H), δ 3.32-3.34 (m, 2H), δ 3.49-3.53 (m, 4H), δ 4.35-4.39 (m, 2H), δ 4.91-4.96 (m, 4H), δ 6.88-6.90 (d, 1H), δ 6.96 (s, 1H), δ 7.30 (s, 1H), δ 7.38-7.42 (m, 2H), δ 7.66 (s, 1H), δ 7.73-7.76 (m, 1H), δ 8.20 (s, 1H).

### Example 132. Synthesis of Compound 132

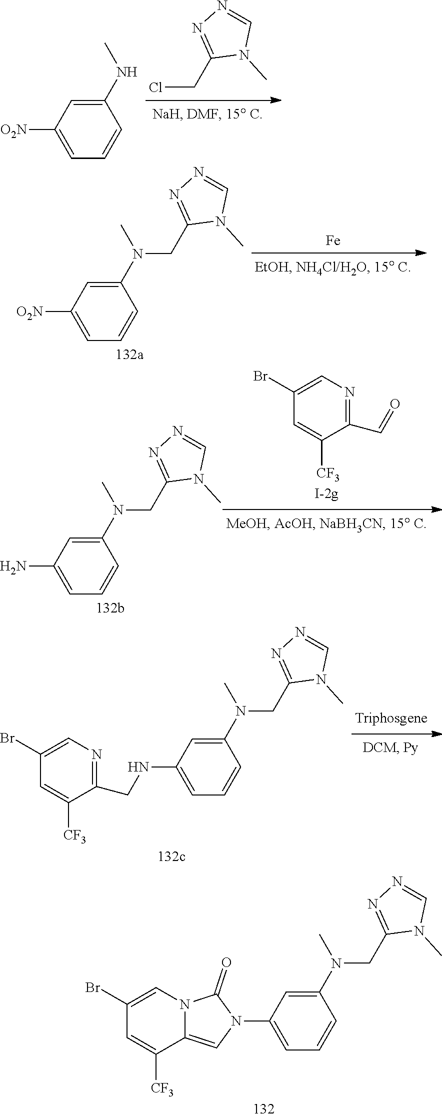

### Synthesis of 132a

To a solution of N-methyl-3-nitro-aniline (500 mg, 3.29 mmol) in DMF (10 mL) was added NaI (394.31 mg, 9.86 mmol, 60% purity) slowly at 15° C. under N₂. The mixture was stirred at 15° C. for 0.5 h. Then to the mixture was added 3-(chloromethyl)-4-methyl-1,2,4-triazole (1.10 g, 6.57 mmol, as a hydrochloride salt) slowly at 15° C. under N₂. The mixture was stirred at 50° C. for 3.5 h. The reaction mixture was quenched by slow addition of saturated aqueous ammonium chloride solution (30 ml) under N₂ and stirred for another 10 min after addition. The mixture was poured into water (20 nil) and extracted with EtOAc (2*30 ml). The organic phase was dried over anhydrous sodium sulfate, filtered, and the filtrate was concentrated under reduced pressure to give a residue. The residue was purified by normal phase SiO₂ chromatography (0-30% ethyl acetate/petroleum ether) to afford compound 132a (300 mg, 36.92% yield) as a yellow solid,

### Synthesis of 132b

A mixture of 132a (1.1 g, 4.45 mmol) and Fe (1.49 g, 26.69 mmol) in EtOH (20 mL) and NH₄Cl (sat. aq.)/12O (5 mL) was degassed and purged with N₂ 3 times, and then the mixture was stirred at 15° C. for 2 hr under N₂ atmosphere. The reaction mixture was filtered and the filtrate was poured into water (100 ml) and extracted with EtOAc (2*100 ml) The organic phase was dried over anhydrous sodium sulfate, filtered, and the filtrate was concentrated under reduced pressure to give a residue. The residue was purified by normal phase SiO₂ chromatography (0-50% ethyl acetate/petroleum ether) to afford compound 132b (500 mg, 55.30% yield) as a yellow solid

### Synthesis of 132c

To a mixture of 132b (500 mg, 2.30 mmol) and I-2g (701.44 mg, 2.76 mmol) in MeOH (10 mL) and CH₃COOH (0101 mL) was added NaBH₃CN (433.85 mg, 6.90 mmol). The mixture was purged with N₂ 3 times, and then the mixture was stirred at 15° C. for 12 hr under N₂ atmosphere. The reaction mixture was diluted with saturated aqueous sodium carbonate solution 50 mL and extracted with EtOAc (2*30 ml). The combined organic layers were dried with anhydrous sodium sulfate, filtered, and the filtrate was concentrated. The residue was purified by normal phase SiO₂ chromatography (0-60% CH2Cl2/methyl alcohol) to afford Compound 132c (250 mg, 24.62% yield) as a yellow solid.

### Synthesis of 132

To a mixture of 132e (200 mg, 439.30 umol) and pyridine (208.49 mg, 2.64 mmol, 212.74 uL) in DCM (10 mL) was added TRIPHOSGENE (130.36 mg, 439.30 umol) at 0° C., and then the mixture was stirred at 15° C. for 4 hr under N₂ atmosphere. The reaction mixture was diluted with saturated aqueous sodium carbonate solution (10 mL) and extracted with DCM (2*30 ml). The combined organic layers were dried with anhydrous sodium sulfate, filtered, and the filtrate was concentrated. The residue was purified by normal phase SiO₂ chromatography (0-60% CH2Cl2/methyl alcohol) to afford Compound 132 (38.9 mg, 16%) was obtained as a yellow solid.

LC-MS: (ES, m/z): [M+H]⁺: 481

H-NMR (400 MHz, DMSO): δ 8.39 (s, 1H), 8.02 (s, 1H), 7.40 (s, 1H), 7.28 (t, J=8.0 Hz, 1H), 7.19 (s, 1H), 7.15 (s, 1H), 7.11 (d, J=8.0 Hz, 1H), 6.89-6.88 (m, 1H), 4.74 (s, 2H), 3.61 (s, 3H), 2.98 (s, 3H).

### Example 133. Synthesis of Compound 133

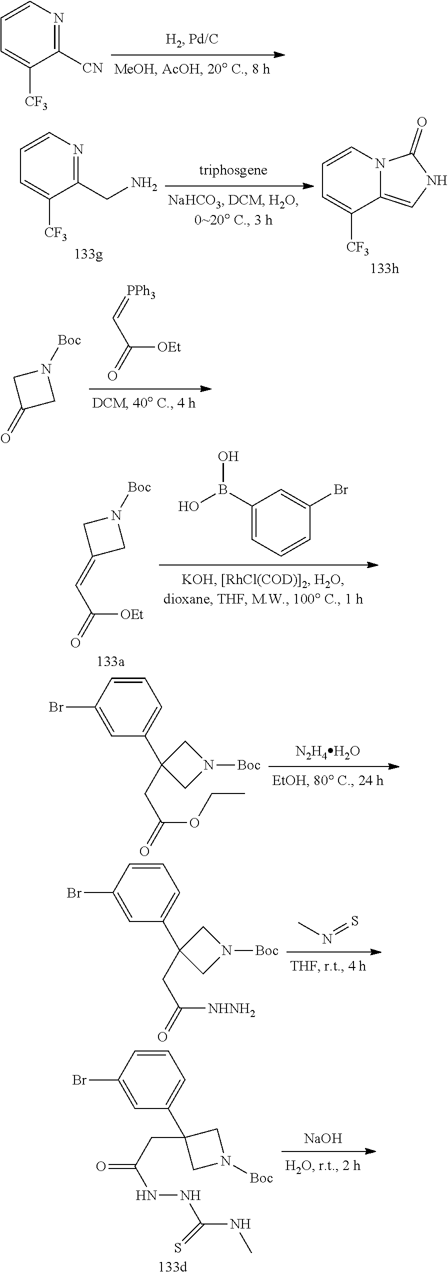

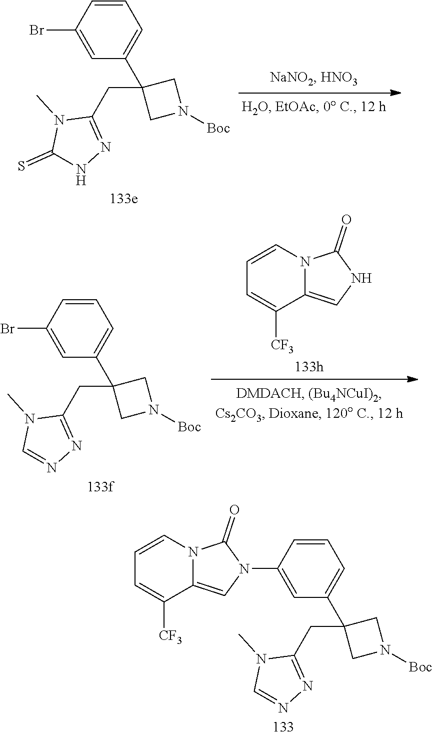 [SMILES: Cn1c(CC2(c3cccc(Br)c3)CN(C(=O)OC(C)(C)C)C2)n[nH]c1=S ; Cn1cnnc1CC1(c2cccc(Br)c2)CN(C(=O)OC(C)(C)C)C1 ; O=c1[nH]cc2c(C(F)(F)F)cccn12 ; Cn1cnnc1CC1(c2cccc(-n3cc4c(C(F)(F)F)cccn4c3=O)c2)CN(C(=O)OC(C)(C)C)C1]

### Synthesis of 133a

To a solution of tert-butyl 3-oxoazetidine-1-carboxylate (10.0 g, 58.0 mmol) in DCM (4 mL) was added ethyl 2-(triphenyl-λ5-phosphanylidene)acetate (22.4 g, 64.3 mmol). The mixture was stirred at 40° C. for 4 hr. The mixture was concentrated in vacuum. The residue was diluted with water (20 mL) and the mixture was filtered. The filtrate was extracted with DCM (50 mL×3). The combined organic layers were dried over anhydrous sodium sulfate, filtered, and concentrated in vacuum. The residue was purified by normal phase SiO2 chromatography (0-20% EtOAc/petroleum ether) to afford 133a as a yellow oil. (14 g, 95% yield),

### Synthesis of 133b

To a solution of chlororhodium;(1Z,5Z)-cycloocta-1,5-diene (49.0 ng, 1.00 mmol) in dioxane (1 mL) was added 1.5 V aqueous KOH solution (4.42 mL) in water (2 mL). After stirring for 5 min, 133a (800 mg, 3.30 mmol) and (3-bromophenyl)boronic acid (1.30 g, 6.60 mmol) in THF (8 mL) was added to the mixture and stirred at 100° C. for 1 hr under microwave. The mixture was concentrated to give the residue. To the residue was added water (30 ml) and saturated aqueous brine solution (20 ml). The mixture was extracted with EtOAc (2×20 ml). The combined organic phase was dried with anhydrous sodium sulfate, filtered, and concentrated to give the residue. The residue was purified by normal phase SiO₂ chromatography (0-15% EtOAc/petroleum ether) to afford 133b as yellow oil (5.4 g, 45% yield).

### Synthesis of 133c

To a solution of 133b (4.20 g, 10.6 mmol) in ethanol (40 mL) was added hydrazine hydrate (8 mL, 161 mmol, 98% purity). It was stirred at 80° C. for 24 hr. The resulting mixture was diluted with water (30 mL). The aqueous layer was extracted with DCM/MeOH (10/1) (5×50 ml). The organic phase was dried with anhydrous sodium sulfate, filtered, and concentrated to afford 133c as a white solid. (2.8 g, 52% yield).

### Synthesis of 133d

To a solution of 133c (3.30 g, 8.50 mmol) in THF (40 mL) was added methylimino(thioxo)methane (1.20 g, 17.0 mmol). The mixture was stirred at r.t. for 4 hr. To the mixture was added water (20 mL) and filtered. The filter cake was concentrated by vacuum to afford 133d as a yellow solid. (5 g, crude).

### Synthesis of 133e

To a solution of NaOH (3.50 g, 87.4 mmol) in water (90 mL) was added 133d (5 g, 11 mmol). The mixture was stirred at r.t. for 2 hr. The resulting mixture was diluted with water (10 mL). The mixture was acidified to pH=5 with 1 N HC solution in H₂O. The aqueous layer was extracted with EtOAc (50 mL). The organic phase was dried with anhydrous sodium sulfate, filtered, and concentrated to afford 133e as a yellow solid (3.5 g, 59% yield).

### Synthesis of 133f

To a solution of 133e (3.5 g, 8.0 mmol) and sodium nitrite (5.0 g, 80 mmol) in water (30 mL) and EtOAc (5 mL) was added 1 M aqueous HNO3 (80 mL) dropwise at 0° C. The mixture was stirred at r.t. for 12 hr. The reaction was quenched by the addition of saturated aqueous sodium bicarbonate solution (100 mL) at r.t. The aqueous layer was extracted with EtOAc (100 mL). The resulting mixture was concentrated under reduced pressure to afford 133f as a yellow solid. (2.5 g, 63% yield)

### Synthesis of 133g

To a solution of 3-(trifluoromethyl)pyridine-2-carbonitrile (1 g, 5.81 mmol) in the mixture of MeOH (6 mL) and HOAc (2 mL) was added palladium on carbon (10 wt, 10%, 0.5 g). The suspension was degassed under vacuum and purged with 112 several times. The mixture was stirred under H2 (15 psi) at 20° C. for 8 h. The reaction mixture was filtered through celite, the filtrate was concentrated in vacuum to afford 133g (1.8 g, crude, acetate) as a green solid,

### Synthesis of 133h

To a solution of sodium bicarbonate (1.66 g, 19.8 mmol) in water (10.8 m-L) was added a solution of TRIPHOSGENE (1.5 g, 5.05 mmol) in DCM (9 mL). Then a solution of 133g (1.8 g, 7.62 mmol, acetate) in DCM (18 mL) was added at 0° C. Then the resulting mixture was slowly warmed to 20° C. and stirred for 3 h. The reaction mixture was poured into water (30 mL) and stirred for 10 min. The aqueous phase was extracted with DCM (30 ml, ×2). The combined organic layers were dried over anhydrous sodium sulfate, filtered, and concentrated in vacuum. The residue was purified by normal phase SiO₂ chromatography (0-50% EtOAc/petroleum ether) to afford 133h (800 mg, 52% yield) as a yellow solid.

### Synthesis of 133

To a solution of 133f (100 mg, 0.25 mmol) and 133h (59.6 mg 030 mmol) in dioxane (8 mL) was added cesium carbonate (160 mg, 0.50 mmol) and iodocopper;tetrabutylammonium;diiodide (55.0 mg, 0.49 mmol,) and (1R,2R)—N1,N2-dimethylcyclohexane-1,2-diamine (14 mg, 0.98 mmol). The mixture was stirred at 120° C. for 12 hr under N2 in a glove box. To the mixture was added water (5 mL). The mixture was extracted with ethyl acetate (2×5 mL). The combined organic phase was washed with saturated aqueous brine solution (2×5 mL), dried with anhydrous sodium sulfate, filtered, and concentrated to give the residue. The residue was purified by reverse phase HPLC to afford Compound 133 as a yellow solid. (11.9 mg, 8%),

LCMS: (ES, m/z): [M+H]⁺ 529

H-NMR: H NMR (400 MHz, DMSO, ppm): δ 8.22 (s, 1H), 7.79 (t, J=7.6 Hz, 2H), 7.52-7.29 (m, 3H), 7.12 (d, J=6.8 Hz, 1H), 6.92 (d, J=8.0 Hz, 1H), 6.30 (t, J=7.2 Hz, 1H), 4.45-4.11 (m, 4H), 3.40 (s, 2H), 3.33 (s, 3H), 2.89 (s, 3H), 1.41 (s, 9H)

### Example 134. Synthesis of Compound 134

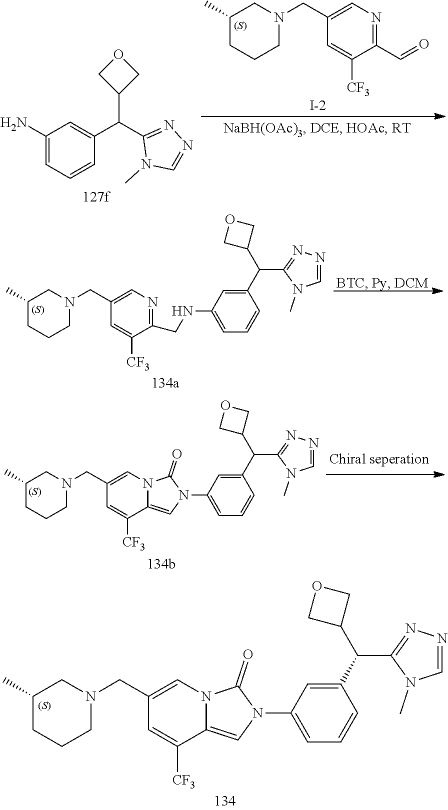

### Synthesis of 134a

Into a 8 mL vial were added 127f (300 mg, 1.228 mmol, 1 equiv), DCE (3 mL), 1-2 (703.15 mg, 2.456 mmol, 2 equiv), NaBH(OAc)3 (780.80 mg, 3.684 mmol, 3 equiv), and HOAc (147.49 mg, 2.456 mmol, 2 equiv) at room temperature. The resulting mixture was stirred overnight at room temperature under nitrogen atmosphere. The resulting mixture was concentrated under vacuum. The residue was purified by Prep-TLC (PE/EA 1:1) to afford 134a (500 mg, 79.12%) as a white solid.

### Synthesis of 134b

To a stirred mixture of 134a (490 mg, 0,952 mmol, 1 equiv) and pyridine (451.91 mg, 5,712 mmol, 6 equiv) in DCM (10 mL) was added Triphosgene (98.90 mg, 0.333 mmol, 0.35 equiv) at 0° C. The resulting mixture was stirred for 1h at 0° C. The reaction was quenched by the addition of sat. NaHCO₃ (aq.) (10 mL) at room temperature. The aqueous layer was extracted with CH₂Cl₂/MeOH (10:1) (3×10 mL). The resulting mixture was concentrated under reduced pressure. The residue was purified by Prep-TLC (CH₂Cl₂/MeOH 12:1) to afford 134b (260 mg, 49.50%) as a yellow solid.

### Synthesis of 134

The 134b (260 mg) was purified by chiral separation with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min: Gradient: 30% B to 30% B in 17 min; Wave Length: 220/254 nm; RT2 (min): 1393) to afford Compound 134 (101.9 mg, 38.80%) as a yellow solid,

LCMS: (ES, m/z): [M+H]⁺ 541

H-NMR: H NMR (400 MHz, DMSO, ppm): δ 0.82-0.84 (m, 4H), δ 1.58-1.63 (m, 5H), δ 1.81-1.98 (m, 1H), δ 2.68-2.80 (m, 2H), δ 3.28 (s, 2H), δ 3.38 (s, 3H), δ 3.88-4.02 (m, 1H), δ 4.27-4.29 (m, 1H), δ 4.48-4.51 (m, 2H), δ 4.72-4.82 (m, 2H), δ 7.01 (s, 1H), δ 7.17-7.20 (d, 1H), δ 7.38 (s, 1H), δ 7.46-7.49 (m, 1H), δ 7.65 (s, 1H), δ 7.75-7.78 (t, 2H), δ 8.38 (s, 1H).

### Example 135. Synthesis of Compound 135

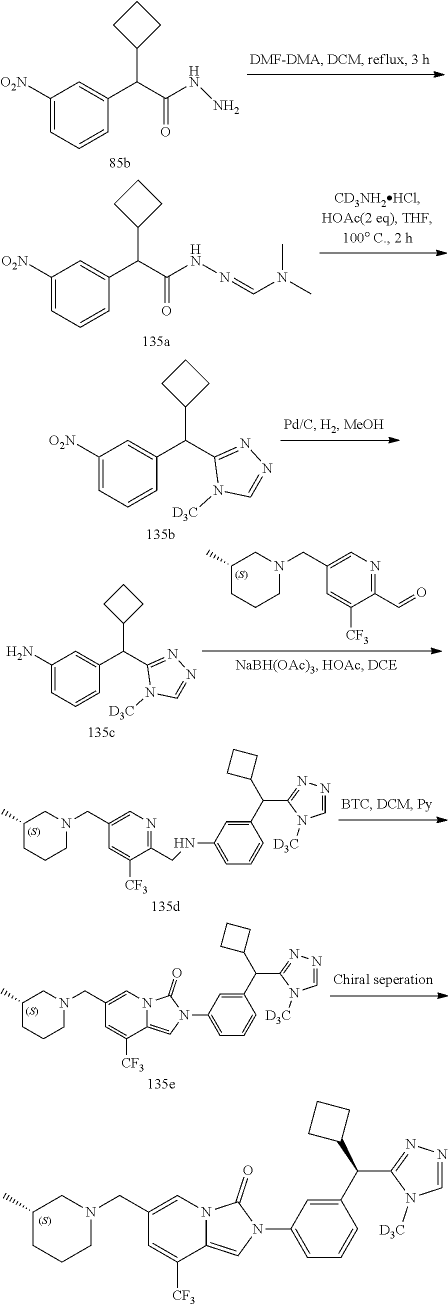

### Synthesis of 135a

To a stirred solution of 85b (8 g, 32.094 mmol, 1 equiv) in DCM (100 mL) was added DMF-DMA (19.12 g, 160.454 mmol, 5.00 equiv) at room temperature under nitrogen atmosphere. The resulting mixture was stirred for 3h at 50° C. under nitrogen atmosphere. The reaction mixture was concentrated under reduced pressure. The residue was purified by silica gel column chromatography, eluted with DCM/MeOH (20:1) to afford 135a (6 g, 59.58%) as a light yellow solid.

### Synthesis of 135b

To a stirred solution of 135a (6 g, 19.714 mmol, 1 equiv) and CD₃NH₂·HCl (4.17 g, 59.142 mmol, 3 equiv) in THF (60 mL) was added HOAc (2.37 g, 39.428 mmol, 2 equiv) at room temperature under nitrogen atmosphere. The resulting mixture was stirred for 2h at 100° C. under nitrogen atmosphere. The resulting mixture was diluted with water (200 mL). The aqueous layer was extracted with EtOAc (3×100 mL). The organic layers were concentrated under reduced pressure. The residue was purified by silica gel column chromatography, eluted with DCM/MeOH (40:1) to afford crude product. The residue was purified by reverse flash chromatography with the following conditions: column, C18 silica gel; mobile phase, MeCN in water, 0% to 50% gradient in 20 min; detector, UV 254 nm. This resulted in 135b (1.2 g, 21.00%) as a white solid.

### Synthesis of 135c

To a solution of 135b (1.2 g, 4,358 mmol, 1 equiv) in MeOH (200 mL) was added Pd/C (0.1 g, 10%) under nitrogen atmosphere in a 500 mL round-bottom flask. The mixture was hydrogenated at room temperature overnight under hydrogen atmosphere using a hydrogen balloon, filtered through a Celite pad, and concentrated under reduced pressure. This resulted in 135c (780 mg, 68.57%) as a white solid.

### Synthesis of 135d

To a stirred solution of 135c (380 mg, 1.549 mmol, 1 equiv) and 1-2 (443.43 mg, 1.549 mmol, 1 equiv) in DC. (8 n) was added HOAc (93.01 mg, 1.549 mmol, 1 equiv) and NaBH(OAc)₃ (656.53 mg, 3.098 mmol, 2 equiv) at room temperature under nitrogen atmosphere. The resulting mixture was stirred for 5h at room temperature under nitrogen atmosphere. The reaction was quenched with saturated aqueous NH₄Cl at room temperature. The aqueous layer was extracted with DCM (2×50 mL). The organic layers were concentrated under reduced pressure. The residue was purified by Prep-TLC (DCM/MeOH 15:1) to afford 135d (400 mg, 47.58%) as a light yellow solid.

### Synthesis of 135e

To a stirred solution of 135d (380 mg, 0,737 mmol, 1 equiv) and Pyridine (582.92 mg, 7.370 mmol, 10 equiv) in DCM (10 mL) was added Triphosgene (87.47 mg, 0.295 mmol, 0.4 equiv) at room temperature under nitrogen atmosphere. The resulting mixture was stirred for 10 min at room temperature. The reaction was quenched with NaHCO₃ (aq.) at room temperature. The aqueous layer was extracted with DCM (2×30 mL). The organic layers were concentrated under reduced pressure. The residue was purified by Prep-TLC (DCM/MeOH 15:1) to afford 135e (250 mg, 59.50%) as a yellow solid.

### Synthesis of 135

The 135e (250 mg) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 40% B in 15.5 min; Wave Length: 220/254 nm; RT1 (min): 9.60; RT2 (min): 12.83, the second peak is product) to afford Compound 135 (86.3 mg, 33.48%) as a yellow solid.

LC-MS: (ES, m/z): [M+H]⁺ 542

H-NMR: (400 MHz, DMSO-d₆, 3 ppm): 0.82-0.83 (m, 4H), 1.41-1.51 (m, 1H), 1.55-1.66 (m, 5H), 1.77-1.93 (m, 5H), 2.05-2.15 (m, 1H), 2.73-2.75 (m, 2H), 3.25-3.32 (m, 3H), 4.24-4.27 (d, 1H), 7.00 (s, 1H), 7.18-7.20 (m, 1H), 7.30 (s, 1H), 7.44-7.46 (m, 1H), 7.65-7.68 (m, 1H), 7.68-7.70 (d, 1H), 7.70-7.74 (d, 1H), 8.32 (s, 1H).

### Example 136. Synthesis of Compound 136

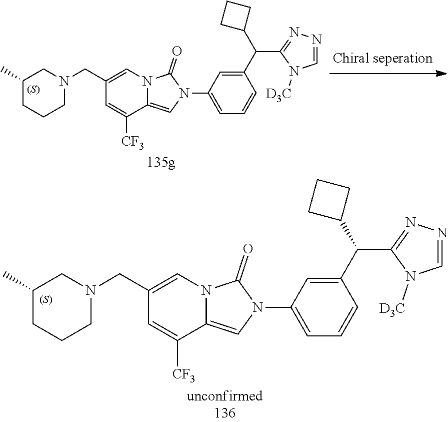

### Synthesis of 136

The 135g (250 mg) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 40% B in 15.5 min; Wave Length: 220/254 nm; RT1 (min): 9.60; RT2 (min): 12.83, the first peak is product) to afford Compound 136 (82.3 mg, 31.93%) as a yellow solid.

LC-MS: (ES, m/z): [M+H]⁺ 542

H-NMR: (400 MHz, DMSO-d₆, 3 ppm): 0.82-0.84 (m, 4H), 1.45-1.55 (m, 1H), 1.59-1.70 (m, 5H), 1.72-1.93 (m, 5H), 2.05-2.15 (m, 1H), 2.73-2.75 (m, 2H), 3.25-3.32 (m, 3H), 4.24-4.27 (d, 1H), 7.01 (s, 1H), 7.19-7.20 (m, 1H), 7.31 (s, 1H), 7.42-7.46 (m, 1H), 7.65-7.68 (m, 1H), 7.68-7.70 (d, 1H), 7.70-7.73 (d, 1H), 8.32 (s, 1H).

### Example 137. Synthesis of Compound 137

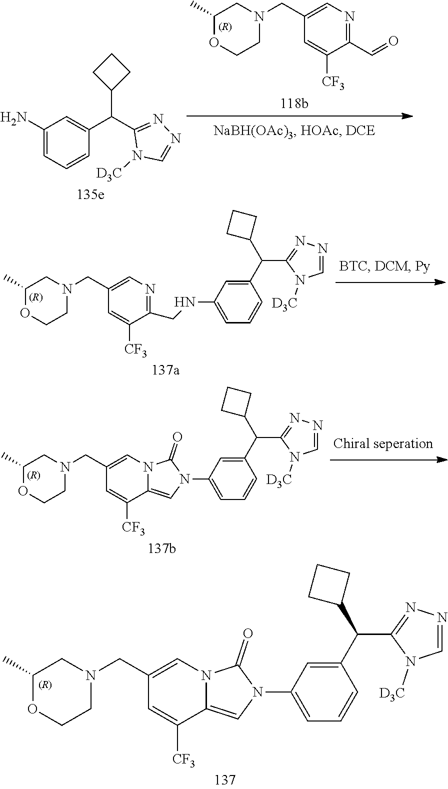

### Synthesis of 137a

To a stirred solution of 135e (380 mg, 1.549 mmol, 1 equiv) and 118b (446.49 mg, 1.549 mmol, 1 equiv) in DCE (8 mL) was added STAB (656.53 mg, 3.098 mmol, 2 equiv) and HOAc (93.01 ng, 1.549 mmol, 1 equiv) at room temperature under nitrogen atmosphere. The resulting mixture was stirred for 5 h at room temperature. The reaction was quenched with saturated aqueous NH₄Cl at room temperature. The aqueous layer was extracted with DCM (2×20 mL). The organic layers were concentrated under reduced pressure. The residue was purified by Prep-TLC (DCM/MeOH 12:1) to afford 137a (280 mg, 34.23%) as a yellow solid.

### Synthesis of 137b

To a stirred solution of 137a (380 mg, 0.734 mmol, 1 equiv) and Pyridine (580.70 mg, 7.340 mmol, 10 equiv) in DCM (10 mL) were added Triphosgene (87.14 mug, 0.294 mmol, 0.4 equiv) at room temperature under nitrogen atmosphere. The resulting mixture was stirred for 10 min at room temperature. The reaction was quenched by the addition of NaHCO₃ (aq.) (10 mL) at room temperature. The aqueous layer was extracted with DCM (2×20 mL). The organic layers were concentrated under reduced pressure. The residue was purified by Prep-TLC (DCM/MeOH 20:1) to afford 137b (280 mg, 68.76%) as a yellow solid.

### Synthesis of 137

The 137b (280 tug) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 17.5 min; Wave Length: 220/254 nm; RT1 (min): 7.02; RT2 (min): 12.54, the second peak is product) to afford Compound 137 (82.5 mg, 29.46%) as a yellow solid,

LC-MS: (ES, m/z): [M+H]⁺ 544

H-NMR: (400 MHz, DMSO-d₆, 3 ppm): 1.03-1.05 (d, 3H), 1.65-1.79 (m, 6H), 1.99-2.13 (m, 2H), 2.58-2.81 (m, 2H), 3.21-3.32 (m, 3H), 3.41-3.52 (m, 2H), 3.71-3.82 (m, 1H), 4.24-4.27 (d, 1H), 7.02 (s, 1H), 7.19-7.20 (m, 1H), 7.31 (s, 1H), 7.42-7.46 (m, 1H), 7.68-7.73 (m, 3H), 8.32 (s, 1H).

### Example 138. Synthesis of Compound 138

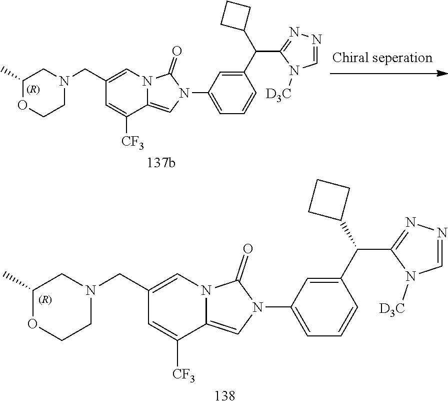

### Synthesis of 138

The 137b (280 mg) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 17.5 min; Wave Length: 220/254 nm; RT1 (min): 7.02; RT2 (min): 12.54, the first peak is product) to afford Compound 138 (751 mg, 26.02%) as a yellow solid.

LC-MS: (ES, m/z): [M+H]⁺ 544

H-NMR: (400 MHz, DMSO-d₆, 3 ppm): 1.03-1.05 (d, 3H), 1.65-1.79 (m, 6H), 1.99-2.13 (m, 2H), 2.58-2.81 (m, 2H), 3.21-3.32 (m, 3H), 3.41-3.52 (m, 2H), 3.71-3.82 (m, 1H), 4.24-4.27 (d, 1H), 7.02 (s, 1H), 7.19-7.20 (m, 1H), 7.31 (s, 1H), 7.42-7.46 (m, 1H), 7.68-7.73 (m, 3H), 8.32 (s, 1H).

### Example 139. Synthesis of Compound 139

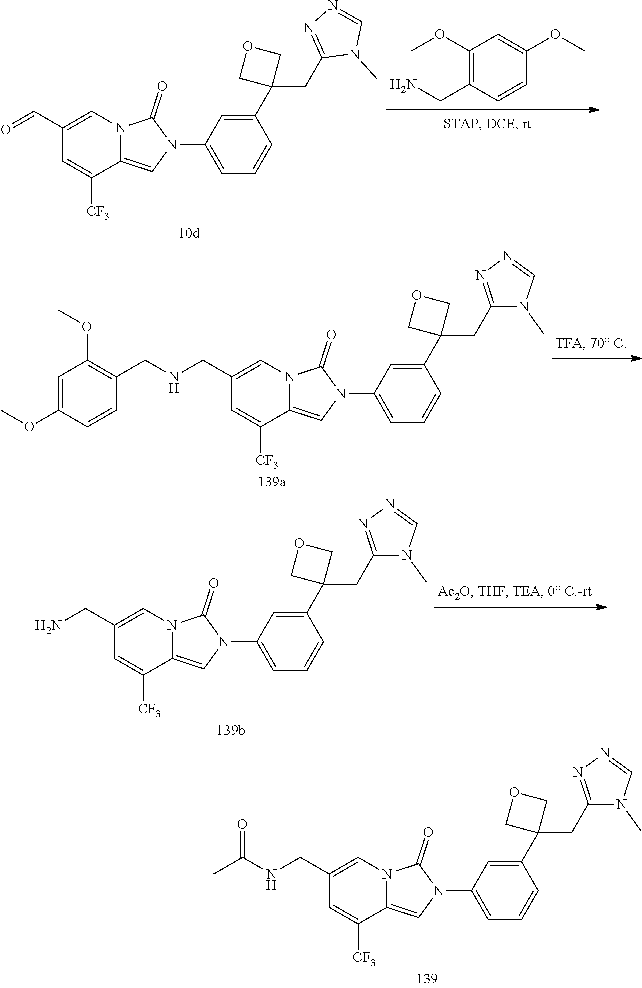

### Synthesis of 139a

To a stirred solution of 10d (300 mg, 0.65 mmol, 1.0 equiv) and 1-(2,4-dimethoxyphenyl) methanamine (109 mg, (0.65 mmol, 1.0 equiv) in DCE (4 ml) was added STAB (278 mg, 1.31 mmol, 2.0 equiv) at room temperature. The resulting mixture was stirred overnight at room temperature. The reaction was quenched with sat, NH₄Cl (aq.) (10 ml) at room temperature. The resulting mixture was extracted with CH₂Cl₂ and MeOH (10:1) (3×10 mL). The combined organic layers were concentrated under reduced pressure. The residue was purified by Prep-TLC (CH₂Cl₂/MeOH=10:1) to afford 139a (150 mg 38%) as a yellow solid.

### Synthesis of 139b

Into an 8 mL sealed tube were added 139a (130 mg, 0.21 mmol, 1.0 equiv) and TFA (1.3 mL) at room temperature. The resulting mixture was stirred for 1 h at 70° C. under nitrogen atmosphere. The resulting mixture was concentrated under reduced pressure. This resulted in 139b (80 mg, 33%) as a brown oil.

### Synthesis of 139

To a stirred solution of 139b (200 mg, 0.43 mmol, 1.0 equiv) and acetic anhydride (9 mg, 0.09 mmol, 0.2 equiv) in THF (3 ml) was added TEA (88 mg, 0.87 mmol, 2.0 equiv) at 0° C. under nitrogen atmosphere. The resulting mixture was stirred for 1 h at 0° C. under nitrogen atmosphere. The reaction was quenched with sat. NH₄Cl (aq.) (10 ml) at room temperature. The resulting mixture was extracted with CH₂Cl₂ (3×10 mL). The resulting mixture was concentrated under reduced pressure. The residue was purified by Prep-TLC (CH₂Cl₂/MeOH=8:1). The crude product (100 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 11% B to 36% B in 8 min; Wave Length: 220 nm; RT1 (min): 7.35) to afford Compound 139 (11 mg, 5%) as a yellow solid,

LC-MS: (ES, m/z): [M+H]⁺ 501

H-NMR: (400 MHz, dmso-d6, δ ppm): 1.88 (s, 3H), 2.91-2.97 (m, 3H), 3.53 (s, 2H), 4.06-4.08 (m, 2H), 4.91-4.96 (m, 4H), 6.89-6.90 (d, 1H), 7.02 (s, 1H), 7.33 (s, 1H), 7.38-7.42 (m, 2H), 7.65 (s, 1H), 7.74-7.77 (m, 1H), 8.20 (s, 1H), 8.30-8.33 (m, 1H).

### Example 140. Synthesis of Compound 140

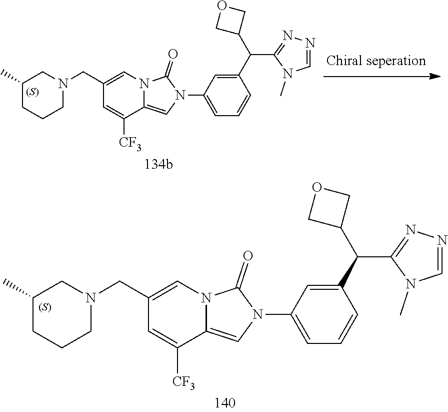

### Synthesis of 140

134b (260 mg) was purified by chiral separation with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 30% B to 30% B in 17 min; Wave Length: 220/254 nm; RT1 (min): 11.36) to afford Compound 140 (95.2 mg) as a yellow solid.

LCMS: (ES, m/z): [M+H]⁺ 541

H-NMR: H NMR (400 MHz, DMSO, ppm): δ 0.82-0.84 (m, 4H), δ 1.58-1.79 (m, 5H), δ 1.81-1.98 (m, 1H), δ 2.63-2.80 (m, 2H), δ 3.25 (s, 2H), δ 3.39 (s, 3H), δ 3.85-4.00 (m, 1H), δ 4.27-4.31 (m, 1H), δ 4.48-4.51 (d, 2H), δ 4.72-4.82 (m, 2H), δ 7.01 (s, 1H), δ 7.17-7.20 (d, 1H), δ 7.38 (s, 1H), δ 7.44-7.49 (m, 1H), δ 7.65 (s, 1H), δ 7.75-7.78 (m, 2H), δ 8.38 (s, 1H).

### Example 141. Synthesis of Compound 141

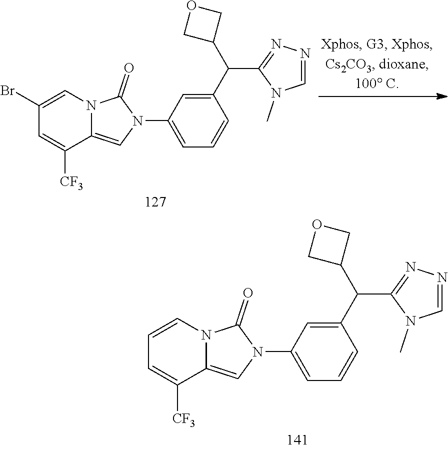 [SMILES: Cn1cnnc1C(c1cccc(-n2cc3c(C(F)(F)F)cc(Br)cn3c2=O)c1)C1COC1 ; Cn1cnnc1C(c1cccc(-n2cc3c(C(F)(F)F)cccn3c2=O)c1)C1COC1]

### Synthesis of 141

To a stirred solution of 127 (40 mg, 0.079 mmol, 1 equiv) and Cs₂CO₃ (76.92 ng, 0.237 mmol, 3 equiv) in dioxane (2 mL) were added XPhos Pd G3 (6.66 mg, 0,008 mmol, 0.1 equiv) and Xphos (7.50 mg, 0,016 mmol, 0.2 equiv) at room temperature under nitrogen atmosphere. The resulting mixture was stirred overnight at 100° C. under nitrogen atmosphere. The reaction was quenched by the addition of water (10 mL) at room temperature. The resulting mixture was extracted with EtOAc (3×6 mL) and dried over anhydrous Na₂SO₄. After filtration, the filtrate was concentrated under reduced pressure. The crude product was purified by reverse phase flash with the following conditions (Column: C18 silicagel column 40 g; Mobile Phase A: Water (0.1% NH₄HCO₃), Mobile Phase B: ACN; Flow rate: 80 mL/min; Gradient: 40% B to 60% B in 14 min; Wave Length: 220 nm) to afford Compound 141 (5.3 mg, 15.51%) as a white solid.

LC-MS: (ES, m/z): [M+H]⁺:430

H-NMR: (400 MHz, DMSO-d6, ppm): δ 3.42 (s, 3H), δ 3.89-3.95 (m, 1H), δ 4.27-4.30 (m, 1H), δ 4.48-4.50 (d, 2H), δ 4.72-4.81 (m, 2H), δ 6.26-6.30 (m, 1H), δ 7.09-7.10 (d, 1H), S 7.17-7.19 (d, 1H), δ 7.37 (s, 1H), δ 7.45-7.47 (m, 1H), δ 7.75-7.79 (m, 3H), δ 8.37 (s, 1H).

### Example 142. Synthesis of Compound 142

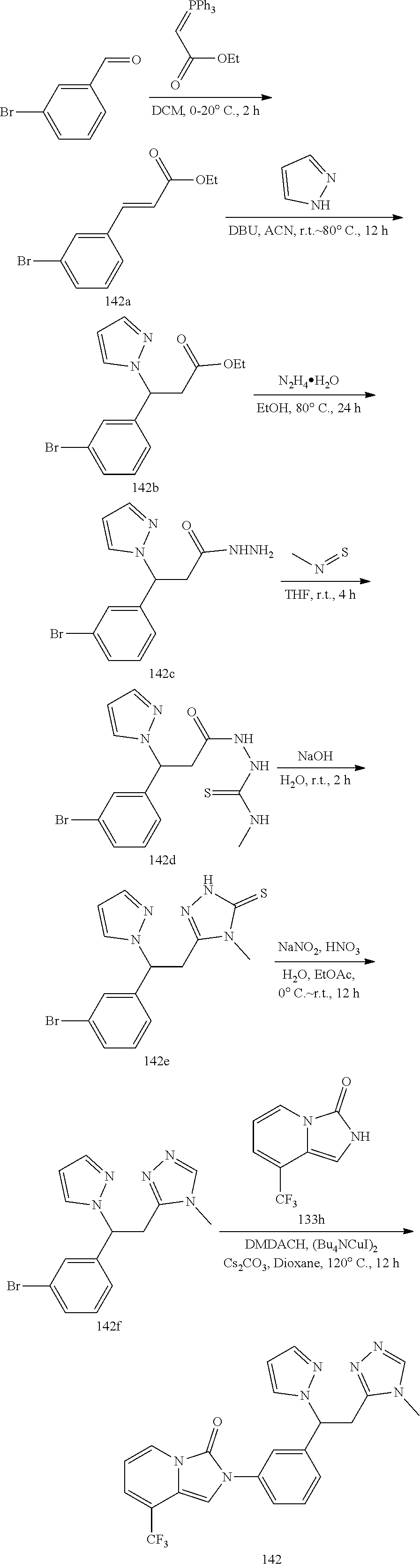

### Synthesis of 142a

To a solution of 3-bromobenzaldehyde (5 g, 27.0 mmol) in DCM (60 mL) was added ethyl 2-(triphenylphosphoranylidene)acetate (9.41 g, 27.0 mmol) at 0° C., then the mixture was stirred at r.t. for 2 h under N₂. The mixture was diluted with water (20 mL), then extracted with EtOAc (2×30 mL). The combined organic layers were dried over anhydrous sodium sulfate, filtered, and the filtrate was concentrated to afford 142a (6.8 g, 98% yield) as a white solid.

### Synthesis of 142b

To a solution of 142a (1 g, 3.92 mmol) and 1H-pyrazole (0.80 g, 11.8 mmol) in MeCN (6 mL) was added a solution of DBU (2.95 mL, 19.6 mmol) in CH3CN (9 mL). Then the mixture was stirred at 50° C. for 5 h under N₂. The mixture was concentrated in vacuum to give a residue, which was further purified by reverse phase HPLC to afford 142b (0.97 g, 76% yield) as a yellow oil.

### Synthesis of 142c

To a solution of 142b (0.8 g, 2.5 mmol) in EtOH (8 mL) was added hydrazine hydrate (1.84 mL, 37.0 mmol, 98% purity). The mixture was stirred at 80° C. for 24 hr. The resulting mixture was diluted with water (30 mL). The aqueous layer was extracted with DCM/MeOH (10/1) (5×50 ml). The organic phase was dried with anhydrous sodium sulfate, filtered, and concentrated to afford 142c (650 mg, 85% yield) as a yellow solid.

### Synthesis of 142d

A solution of 142c (0.40 mg, 1.3 mmol) and methylimino(thioxo)methane (189 mg, 2.6 mmol) in THF (9 mL) was stirred at r.t. for 4 hr. To the mixture was added water (8 ml) and filtered. The filter cake was concentrated by vacuum to afford 142d as a yellow solid (450 mg, 91% yield)

### Synthesis of 142e

To a solution of 142d (450 mg, 1.2 mmol) in H₂O (10 mL) was added sodium hydroxide (470 mg, 11.8 mmol). The mixture was stirred at r.t. for 2 hr. The resulting mixture was diluted with water (5 mL). The mixture was acidified to pH 5 with HCl (1 M). The aqueous layer was extracted with CH₂Cl2/MeOH (10/1) (3×5 mL). The resulting mixture was concentrated under reduced pressure to afford 142e as a yellow solid. (420 mg, 98% yield).

### Synthesis of 142f

To a solution of 142e (420 mg, 1.2 mmol) and NaNO₂ (800 mg, 11.5 mmol) in H₂O (4 mL) and ethyl acetate (1 mL) was added HNO₃ (730 mg, 11.5 mmol) at 0° C. The mixture was stirred at r.t. for 12 hr. The reaction was quenched by the addition of saturated aqueous sodium bicarbonate solution (10 mL) at r.t. The aqueous layer was extracted with ethyl acetate (3×10 mL). The resulting mixture was concentrated under reduced pressure to afford 142f as a yellow solid (220 mag, 57% yield).

### Synthesis of 142

To a solution of 142f (200 mg, 0.6 mmol) and 133h (146 mg, 0.72 mmol) in dioxane (6 mL) was added (1R,2R)—N1,N2-dimethylcyclohexane-1,2-diamine (34.3 mg, 0.24 mmol) and iodocopper;tetrabutylammonium;diiodide (135 mg, 0.12 mmol) and cesium carbonate (392 mg, 1.2 mmol). The mixture was stirred at 120° C. for 12 hr. Water (5 mL) was added to the mixture. The mixture was extracted with ethyl acetate (2×5 mL). The combined organic phase was washed with saturated aqueous brine solution (2×5 mL), dried with anhydrous sodium sulfate, filtered and concentrated to give the residue. The residue was purified by reverse phase HPLC to afford 142 as a yellow solid. (30.2 mg, 10% yield)

LCMS: (ES, m/z): [M+H]⁺ 454.

H-NMR: (400 MHz, DMSO-d₆, ppm): δ 8.33 (br s, 1H), 7.92 (s, 2H), 7.87-7.78 (m, 2H), 7.54-7.43 (m, 2H), 7.40-7.30 (m, 2H), 7.12 (br d, J=6.8 Hz, 1H), 6.30 (t, J=6.8 Hz, 1H), 6.23 (s, 1H), 6.16-6.13 (m, 1H), 4.00-3.85 (m, 1H), 3.74-3.61 (m, 1H), 3.55 (s, 3H)

### Example 143. Synthesis of Compound 143

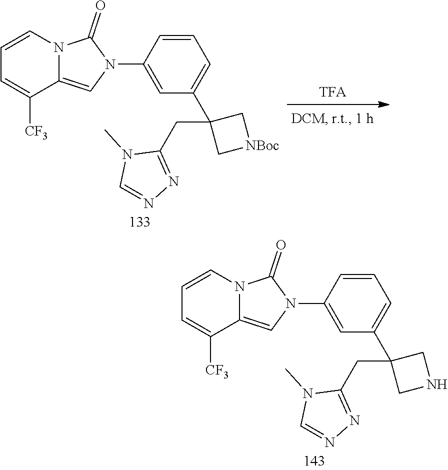 [SMILES: Cn1cnnc1CC1(c2cccc(-n3cc4c(C(F)(F)F)cccn4c3=O)c2)CN(C(=O)OC(C)(C)C)C1 ; Cn1cnnc1CC1(c2cccc(-n3cc4c(C(F)(F)F)cccn4c3=O)c2)CNC1]

### Synthesis of 143

To a solution of 133 (450 mg, 0.85 mmol) in DCM (8 ml) was added trifluoroacetic acid (2.25 mL, 30.4 mmol). The mixture was stirred at r.t. for 1 hr. The mixture was concentrated in vacuum. The residue was purified by reverse phase HPLC to afford Compound 143 (8.4 mg, 1.9%) as a yellow solid.

LCMS: (ES, m/z): [M+H]⁺ 429.

H-NMR: (400 MHz, DMSO-d₆, ppm): δ 8.18 (s, 1H), 7.77 (d, J=7.2 Hz, 1H), 7.75-7.72 (m, 1H), 7.43-7.32 (m, 2H), 7.27 (s, 1H), 7.09 (d, J=6.8 Hz, 1H), 6.80 (d, J=7.6 Hz, 1H), 6.28 (t, J=7.2 Hz, 1H), 4.15-4.01 (m, 4H), 3.46 (s, 3H), 2.84 (s, 3H).

### Example 144. Synthesis of Compound 144

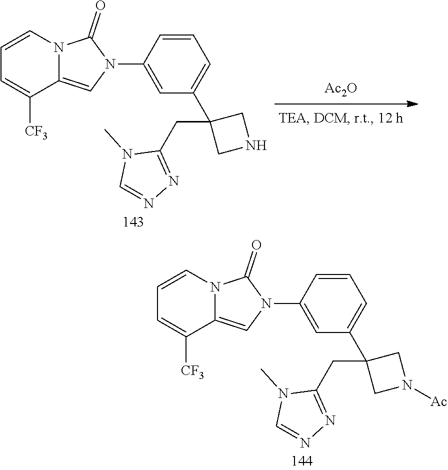

### Synthesis of 144

To a solution of 143 (500 mg, 1.17 mmol) in DCM (5 mL) was added acetic anhydride (0.16 mL, 1.8 mmol) and triethylamine (6.0 mL, 43.1 mmol). The mixture was stirred at r.t. for 12 hr. To the mixture was added water (10 mL). The mixture was extracted with ethyl acetate (2×10 mL). The combined organic phase was dried with anhydrous sodium sulfate, filtered, and concentrated to give the residue. The residue was purified by reverse phase HPLC to afford 144 as a yellow solid, (20.1 mg, 3% yield)

LCMS: (ES, m/z): [M+H]⁺ 471.

H-NMR: (400 MHz, DMSO-d₆, ppm): δ 8.22 (s, 1H), 7.84-7.75 (m, 2H), 7.47 (s, 1H), 7.42 (t, J=8.0 Hz, 1H), 7.36 (s, 1H), 7.12 (d, J=6.4 Hz, 1H), 6.95 (d, J=8.0 Hz, 1H), 6.30 (t, J=6.8 Hz, 1H), 4.60 (d, J=8.4 Hz, 1H), 4.50 (d, J=8.4 Hz, 1H), 4.31 (d, J=9.6 Hz, 1H), 4.19 (d, J=9.6 Hz, 1H), 3.43 (s, 2H), 2.93 (s, 3H), 1.83 (s, 3H)

### Example 145. Synthesis of Compound 145

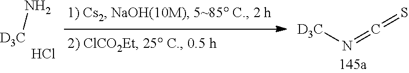

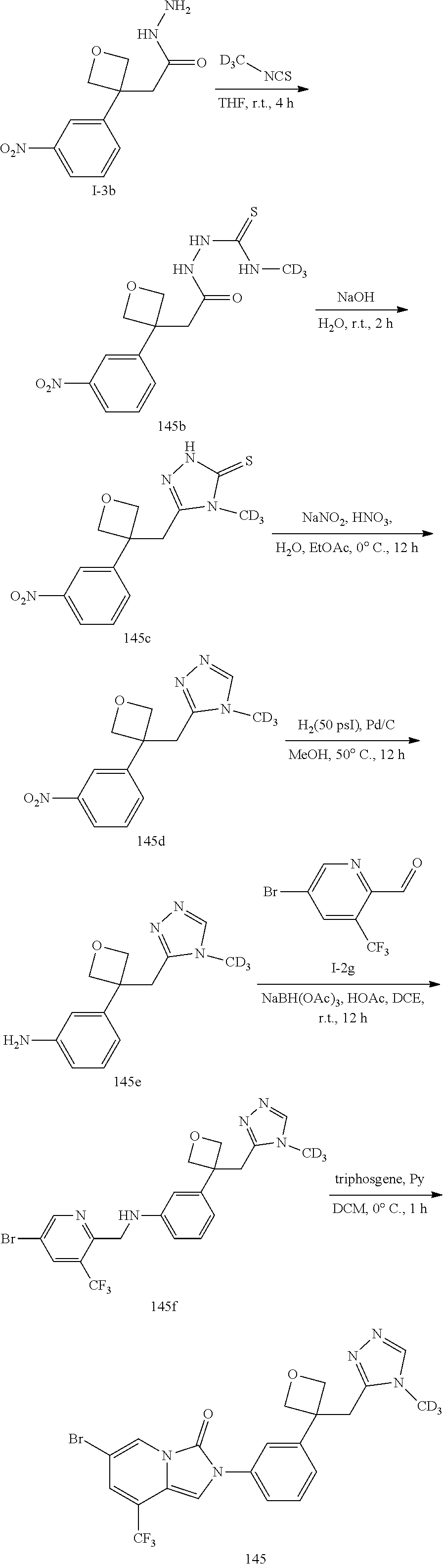

### Synthesis of 145a

To a solution of carbon disulfide (4.28 mL, 70.8 mmol) and NaOH (5.67 g, 141 mmol) in water (15 ml) was added trideuteriomethanamine (5.0 g, 70.8 mmol, as a hydrochloride salt) at 5° C., then the mixture was stirred at 85° C. for 2 h. Then the mixture was cooled to 30° C., ethyl carbonochloridate (7.69 g, 70.89 mmol, 6.75 mL) was added to the mixture, and the resulting mixture was stirred at 25° C. for 0.5 hr. The reaction mixture was standing for 12 h, then diluted with water 40 mL and extracted with DCM (40 mL×3). The combined organic layers were dried over anhydrous sodium sulfate, filtered, and concentrated under reduced pressure to afford 145a (3.5 g, crude) as a yellow oil.

### Synthesis of 145b

To the mixture of I-3b (749 mg, 2.99 mmol) in THY (12 mL) was added 145a (0.5 g, 6.57 mmol) then the mixture was stirred at r.t. for 4 h. The resulting mixture was diluted with water (600 mL). The precipitated solids were collected by filtration and washed with water (3×50 ml) to afford 145b (0.5 g, 51.16% yield) as a yellow solid.

### Synthesis of 145c

To the mixture of NaOH (0.4 g, 10.00 mmol) in water (10 mL) was added 145b (0.4 g, 1.22 mmol), and the mixture was stirred at r.t. for 2 h. The resulting mixture was diluted with water (50 ml) and acidified to pH=5 by 1 N aqueous HCl solution. The aqueous layer was extracted with CH₂C1₂/MeOH (10/1) (20 ml×3). The resulting organic phase was concentrated under reduced pressure to afford 145c (0.5 g, crude) as a yellow solid.

### Synthesis of 145d

HNO₃ (1.07 mL, 16.16 mmol, 68% purity) was added to water (15 mL) to afford 1 N aqueous HNO₃ solution (16 mL). To a stirred solution of 145c (0.5 g, 1.62 mmol) and sodium nitrite (1.12 g, 16.1 mmol) in water (5 mL) and EtOAc (1 mL) was added 1 N aqueous HNO₃ solution (16 mL) dropwise at 0° C. The resulting mixture was stirred at r.t. for 12 h. The reaction was quenched by the addition of saturated aqueous sodium bicarbonate solution (50 mL). The aqueous layer was extracted with DCM (50 mL×3). The combined organic layers were dried over anhydrous sodium sulfate, filtered, and concentrated under reduced pressure to afford 145d (200 mg, crude) as a white solid.

### Synthesis of 145e

To a solution of 145d (70 mg, 252 umol) in MeOH (5 mL) was added palladium on carbon (10 wt, 10%, 50 mg). The mixture was stirred at 50° C. for 12 h under H₂ (50 psi). The mixture was filtered and the filtrate was concentrated under reduced pressure to afford 145e (60 mg, crude) as a yellow oil.

### Synthesis of 145f

To a solution of 145e (60 mg, 242 umol) and I-2g (123 ng, 485 umol) in DCE (2 mL) was added sodium triacetoxyborohydride (102 mg, 485 umol) and acetic acid (41.6 uL, 727 umol). The mixture was stirred at r.t. for 12 h. The mixture was neutralized with saturated aqueous sodium bicarbonate solution (20 ml) and extracted with DCM (3×10 ml). The combined organic layers were washed with water (2×20 ml) and dried over anhydrous sodium sulfate, filtered and the filtrate was concentrated in vacuum to afford 145f (450 mg, crude) as a brown oil.

### Synthesis of 145

To a solution of 145f (400 mg, 824 umol) in DCM (10 mL) was added pyridine (266 uL, 330 mmol) and TRIPHOSGENE (97.8 mg, 329 umol) at 0° C. The mixture was stirred at 0° C. for 1 h under N₂ The reaction was quenched with saturated aqueous sodium bicarbonate solution (20 mL). The aqueous layer was extracted with DCM (3×20 mL). The combined organic layers were dried over anhydrous sodium sulfate, filtered, and the filtrate was concentrated under reduced pressure to give a residue. The residue was purified by reverse phase HPLC to afford 145 (5 mg, 1.12% yield) as a brown solid.

LCMS: (ES, m/z): [M+H]⁺ 511.

H-NMR: (400 MHz, DMSO-d₆, ppm): δ 8.20 (s, 1H), 8.04 (s, 1H), 7.77-7.71 (m, 1H), 7.46-7.38 (m, 3H), 7.18 (s, 1H), 6.91 (d, J=8.0 Hz, 1H), 4.96-4.90 (m, 4H), 3.53 (s, 2H)

### Example 146. Synthesis of Compound 146

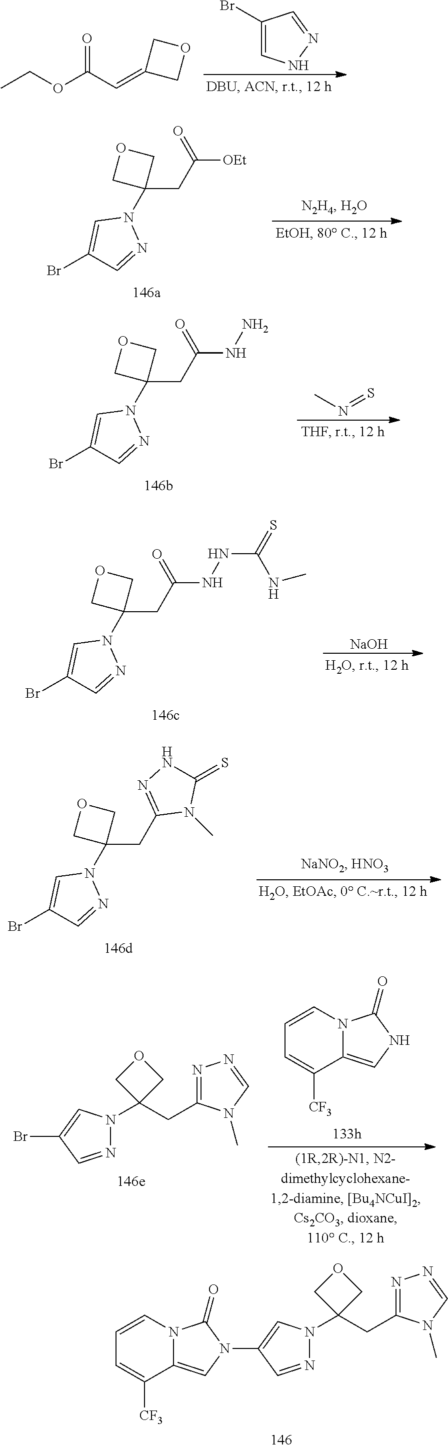

### Synthesis of 146a

To a solution of ethyl 2-(oxetan-3-ylidene)acetate (5 g, 35.17 mmol) in A CN (50 mL) was added DBU (7.95 mL, 52.7 mmol) and 4-bromo-1H-pyrazole (5.69 g, 38.6 mmol). The mixture was stirred at r.t. for 12 h. The reaction mixture was concentrated under reduced pressure to give a residue. The residue was purified by normal phase SiO₂ chromatography (0-20% EtOAc/petroleum ether) to afford 146a (8.6 g, 80% yield) as yellow oil.

### Synthesis of 146b

To a solution of 146a (8.6 g, 29.7 mmol) in EtOH (80 mL) was added N₂H₄·H₂O (14.7 mL, 297 mmol, 98% purity). The mixture was stirred at 80° C. for 12 h. The reaction mixture was concentrated under reduced pressure to remove solvent. The mixture was diluted with water (100 mL) and extracted with DCM (100 mL×3). The combined organic layers were dried over anhydrous sodium sulfate, filtered, and concentrated under reduced pressure to afford 146b (8.7 g, crude) as white solid.

### Synthesis of 146c

To a solution of 146b (8.9 g, 32.3 mmol) in THE (180 mL) was added methylimino(thioxo)methane (4.42 mL, 64.7 mmol). The mixture was stirred at r.t. for 12 b. The reaction mixture was concentrated under reduced pressure to afford 146c (13 g, crude) as a white solid.

### Synthesis of 146d

A solution of 146c (11 g, 31.5 mmol) in 1 N aqueous NaOH solution (252.71 mL) was stirred at r.t. for 12 h. The reaction mixture was diluted with H₂O (200 mL) and adjusted to pH=3 with HCl (1M). The mixture was extracted with DCM (200 mL×3). The combined organic layers were washed by saturated aqueous brine solution (100 mL×2), dried over anhydrous sodium sulfate, filtered and concentrated under reduced pressure to afford 146d (6 g, crude) as a white solid.

### Synthesis of 146e

HNO₃ (8.86 mL, 196 mmol) was added dropwise to water (188 mL) to afford 1 N aqueous HNO₃ solution (196 mL). To a stirred solution of 146d (6.5 g, 19.6 mmol) and sodium nitrite (135 g, 196 mmol) in water (60 mL) and EtOAc (10 mL) was added 1 N aqueous HNO₃ solution (196 mL) dropwise at 0° C. The resulting mixture was stirred at r.t. for 12 h. The reaction was quenched by the addition of saturated aqueous sodium bicarbonate solution (200 mL). The aqueous layer was extracted with CH₂Cl₂/MeOH (10/1) (100 mL×3). The resulting mixture was concentrated under reduced pressure to afford 146e (2.2 g, crude) as a white solid.

### Synthesis of 146

The reaction was run in 50 ng scale with 10 batches in parallel.

A mixture of 146e (50 ag, 167 umol), 133h (37.2 mg, 184 umol), iodocopper;tetrabutylammonium;diiodide (18.7 mg, 16.7 umol), (1R;2R)—N1,N2-dimethylcyclohexane-1,2-diamine (4.77 mg, 33.5 umol) and cesium carbonate (109 mg, 335 umol) in dioxane (1 mL) was stirred at 110° C. for 12 h under N₂ atmosphere. The reaction mixture was filtered and concentrated under reduced pressure to give a residue. The residue was purified by reverse phase HPLC to afford a crude product. NMR showed it contained TPPO, so it was further purified by PCX (polymer strong cation exchange) (MeOH:H₂O 1:1) to afford Compound 146 (35 mg, 4% yield) as a yellow solid.

LCMS: (ES, m/z): [M+H]⁺ 420.

H-NMR: (400 MHz, DMSO-d₆, ppm): δ 8.50 (s, 1H), 8.31 (s, 1H), 8.15 (s, 1H), 7.79 (d, J=6.8 Hz, 1H), 7.37 (s, 1H), 7.10 (d, J=6.4 Hz, 1H), 6.29 (t, J=6.8 Hz, 1H), 5.10-4.94 (m, 4H), 3.75 (s, 2H), 3.29 (s, 3H)

### Example 147. Synthesis of Compound 147

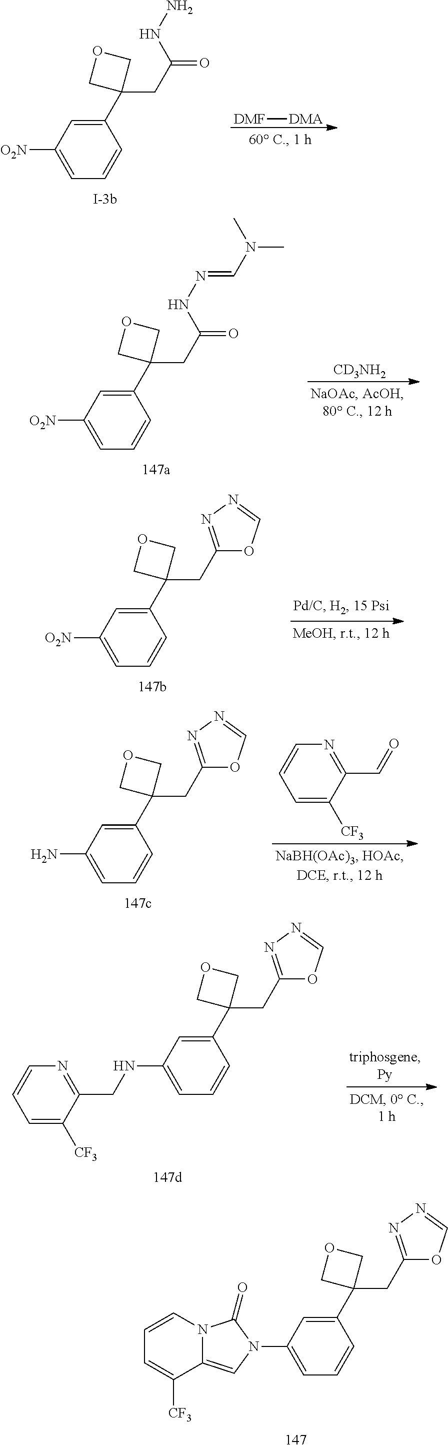

### Synthesis of 147a

A solution of I-3b (1.8 g, 7.16 mmol, 1 eq) in DMF-DMA (16.15 g, 135.50 mmol, 18.00 mL, 18.91 eq) was stirred at 60° C. for 1 br. The reaction mixture was concentrated under reduced pressure to give a crude product 147a (3 g, crude) as brown oil, which was used in the next step without further purification.

### Synthesis of 147b

To a solution of 147a (1.9 g, 6.20 mmol) in AcOH (20 mL) was added sodium acetate (1.02 g, 12.4 mmol) and trideuteriomethanamine (656 mg, 9.30 mmol, as a hydrochloride salt). The mixture was stirred at 80° C. for 12 hr. The reaction mixture was concentrated under reduced pressure to give a residue. The residue was purified by normal phase SiO2 chromatography (0-50% EtOAc/petroleum ether) to afford 147b (210 mg, 13% yield) as a white solid.

### Synthesis of 147c

A mixture of 147b (150 mg, 574 umol) and palladium on carbon (10 wt. 10%, 100 mg) in MeOH (5 mL) was stirred at 20° C. for 12 hr under H₂ (15 psi) atmosphere. The reaction mixture was filtered through celite and the filtrate was concentrated under reduced pressure to afford 147c (90 mg, crude).

### Synthesis of 147d

To a solution of 147c (0.08 g, 345 umol) in DCE (2 mL) was added 3-(trifluoromethyl)pyridine-2-carbaldehyde (60.5 mg, 345 umol), NaOAc (113 mg, 1.38 mmol), and NaBH(OAc)3 (146 mg, 691 umol). The mixture was stirred at 20° C. for 12 h. The mixture was adjusted to pH=8 with saturated aqueous sodium bicarbonate solution and extracted with DCM (5 ml*3). The combined organic layers were dried over anhydrous sodium sulfate, filtered and the filtrate was concentrated under reduced pressure to afford 147d (90 mg, crude) as a yellow oil.

### Synthesis of 147

To a solution of 147d (40.0 mg, 102 umol) in DCM (1 mL) was added pyridine (33.1 uL, 409 umol) and TRIPHOSGENE (121 mg, 40.9 umol) at 0° C. The mixture was stirred at 0° C. for 1 h. The reaction was quenched with saturated aqueous sodium bicarbonate solution (20 ml). The aqueous layer was extracted with DCM (3×20 mL). The combined organic layers were dried over anhydrous sodium sulfate and filtered. The filtrate was concentrated in vacuum. The residue was purified by reverse phase HPLC to afford Compound 147 (13 mg, 28% yield) as a yellow solid.

LCMS: (ES, m/z): [M+H]⁺ 417.

H-NMR: (400 MHz, DMSO-d₆, ppm): δ=9.09 (s, 1H), 7.81 (br d, J=6.8 Hz, 2H), 7.57 (s, 1H), 7.45 (t, J=8.0 Hz, 1H), 7.39 (s, 1H), 7.14-7.06 (m, 2H), 6.30 (t, J=6.4 Hz, 1H), 4.96 (d, J=6.4 Hz, 2H), 4.89 (d, J=6.4 Hz, 2H), 3.75 (s, 2H)

### Example 148. Synthesis of Compound 148

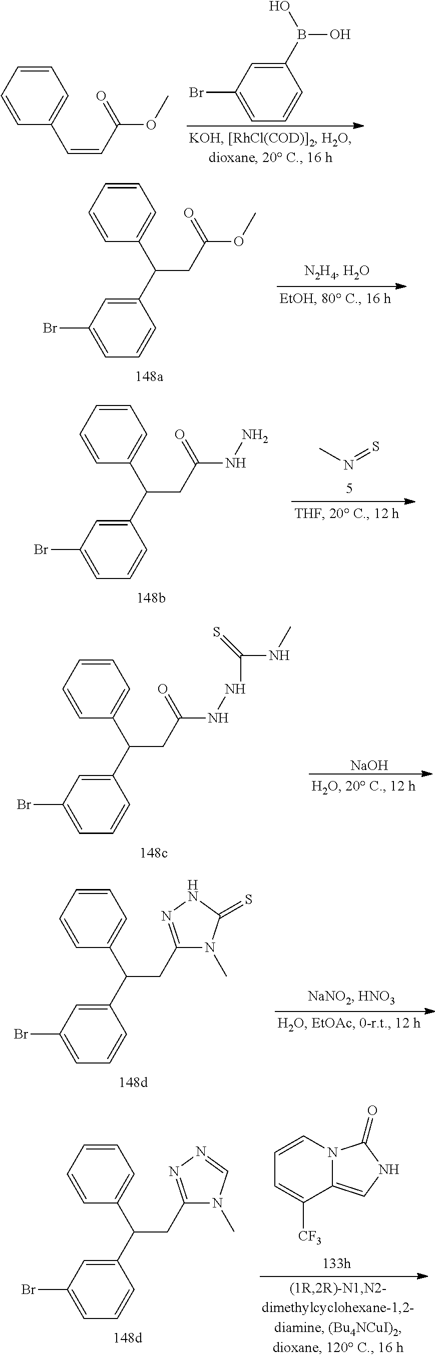

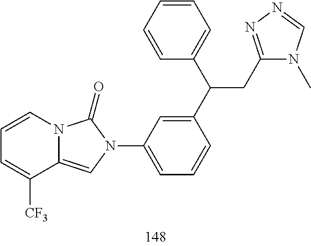 [SMILES: Cn1cnnc1CC(c1ccccc1)c1cccc(-n2cc3c(C(F)(F)F)cccn3c2=O)c1]

### Synthesis of 148a

To a solution of potassium hydroxide (l 38 g, 24.6 mmol) in water (10 mL) and dioxane (30 mL) was added chlororhodium;(1Z,5Z)-cyclocta-1,5-diene (304 mg, 616 umol) at 20° C., After addition, the mixture was stirred at this temperature for 0.5 h, then methyl (Z)-3-phenylprop-2-enoate (2 g, 123 mmol) and (3-bromophenyl)boronic acid (4.95 g, 24.6 mmol) was added. The resulting mixture was stirred at r.t. for 15.5 h. The reaction mixture was concentrated in vacuum to remove the dioxane. Then the residue was diluted with water (20 mL) and extracted with EtOAc (20 mL×2). The combined organic phase was washed with saturated aqueous brine solution (10 mL), dried with anhydrous sodium sulfate, filtered, and concentrated in vacuum. The residue was purified by normal phase SiO₂ chromatography (0-18% EtOAc/petroleum ether) to afford 148a (3.4 g, 86% yield).

### Synthesis of 148b

To a solution of 148a (3.2 g, 10.0 mmol) in EtOH (50 mL) was added NH₂NH₂·H₂O (7.31 mL, 150 mmol). The mixture was stirred at 80° C. for 16 hr. The reaction was concentrated under reduced pressure to give a residue that was then diluted with water (100 mL). The aqueous phase was extracted with ethyl acetate (2×100 mL). The combined organic phase was washed with saturated aqueous brine solution (30 mL), dried with anhydrous sodium sulfate, filtered, and concentrated in vacuum to afford 148b (3.2 g, 100% yield) as a colorless oil.

### Synthesis of 148c

A mixture of 148b (3.2 g, 10.0 mml), methylimino(thioxo)methane (1.37 mL, 20.0 mmol) in THF (30 mL) was stirred at 20° C. for 12 h under N2 atmosphere. The reaction was poured into water (50 mL) and solid formed. The solid was collected and dried to afford 148c (3.9 g, 99% yield) as a white solid.

### Synthesis of 148d

A suspension of 148c (3.9 g, 9.94 mmol) in 1 N aqueous NaOH solution (79.5 mL) was stirred at 20° C. for 12 hr under N2 atmosphere. The resulting mixture was diluted with water (40 mL). The mixture was acidified to pH 5 with HCl (1 M). The aqueous layer was extracted with ethyl acetate (2×80 mL). The combined organic phase was washed with brine (20 mL), dried with anhydrous sodium sulfate, filtered, and concentrated in vacuum to afford 148d (3.4 g, 91% yield) as a white solid.

### Synthesis of 148e

HNO₃ (6.01 mL, 90.8 mmol, 68% purity) was added drop wised to water (80 mL) afford a 1 M aqueous HNO₃ solution (86 mL), To a stirred solution of 148d (3.4 g, 9.08 mmol) and sodium nitrite (6.27 g, 90.8 mmol) in water (60 mL) and EtOAc (20 mL) was added 1 M aqueous HNO₃ solution (86 mL) dropwise at 0° C. The resulting mixture was stirred at r.t. for 12 h. The reaction was poured into saturated aqueous sodium bicarbonate solution (100 mL) and the resulting mixture was extracted with EtOAc (2×100 mL). The organic phase was washed with saturated aqueous brine solution (50 mL), dried over anhydrous sodium sulfate, and concentrated in vacuum to give a residue. The residue was purified by normal phase SiO₂ chromatography (0-10% MeOH/DCM) to afford 148e as a white solid. (2.9 g, 82% yield)

### Synthesis of 148

A mixture of 148e (150 mg, 438 umol), 133h (106 mg, 525 umol), iodocopper;tetrabutylammonium;diiodide (98.1 mg, 87.6 umol), (1R,2R)—N1,N2-dimethylcyclohexane-1,2-diamine (24.9 tug, 175 umol), and cesium carbonate (285.61 mg, 876.60 umol) in dioxane (10 mL) was stirred at 110° C. for 12 h under N2 atmosphere. The mixture was poured into water (60 mL). The aqueous phase was extracted with ethyl acetate (2×50 mL). The combined organic phase was washed with saturated aqueous brine solution (20 mL), dried with anhydrous sodium sulfate, filtered, and concentrated in vacuum. The residue was purified by reverse phase HPLC to afford Compound 148 as a yellow solid. (28.7 mg, 6% yield)

LCMS: (ES, m/z): [M+H]⁺ 464.

H-NMR: (400 MHz, DMSO-d₆, ppm): δ 8.33 (s, 1H), 7.92 (s, 2H), 7.87-7.78 (m, 2H), 7.54-7.43 (m, 2H), 7.40-7.30 (m, 2H), 7.12 (d, J=6.8 Hz, 1H), 6.30 (t, J=6.8 Hz, 1H), 6.23 (s, 1H), 6.16-6.13 (m, 1H), 4.00-3.85 (m, 1H), 3.74-3.61 (m, 1H), 3.55 (s, 3H)

### Example 149. Synthesis of Compound 149

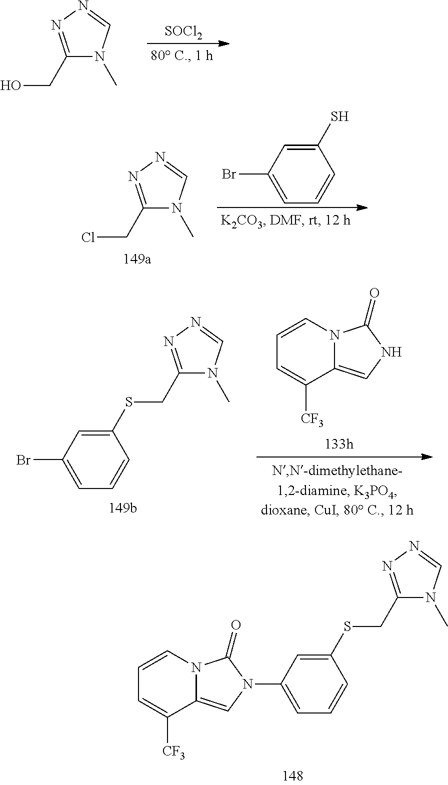

### Synthesis of 149a

A solution of (4-methyl-1,2,4-triazol-3-yl)methanol (1.00 g, 8.84 mmol) in thionyl chloride (10 mL, 137 mmol) was stirred at 80° C. for 1 hr. The reaction mixture was concentrated under reduced pressure to give 149a (1.5 g, crude, hydrochloride salt) as yellow oil.

### Synthesis of 149b

To a solution of 149a (1.50 g, 8.93 mmol, as a hydrochloride salt) in DMF (10 mL) was added potassium carbonate (271 g, 19.6 mmol) and 3-bromobenzenethiol (968 uL, 9.37 mmol). The mixture was stirred at room temperature for 12 hr. The reaction mixture was poured into water (50 mL) and extracted with EtOAc (50 mL×3). The combined organic layers were washed with brine (50 mL×2), dried over anhydrous sodium sulfate, filtered, and concentrated under reduced pressure to give a residue. The residue was purified by normal phase SiO₂ chromatography (0-25% EtOAc/petroleum ether) to afford 149b (1.6 g, 57% yield) as a white solid.

### Synthesis of 149

A mixture of 149b (500 mg, 1.75 mmol), 133h (355 mg, 1.75 mmol), CuI (67.0 mg, 351 umol), N′,N′-dimethylethane-1,2-diamine (38.4 uL, 351 umol) and potassium phosphate (747 mg, 3.51 mmol) in dioxane (20 mL) was stirred at 80° C. for 12 hr under N₂ atmosphere. The reaction mixture was poured into water (50 ml), while yellow solid formed. The solid was filtered and washed by water (2×10 mL). The cake was collected and dried under reduced pressure to give residue. The residue was triturated with MeOH (10 mL) for 30 min, then the mixture was filtered and the filtrate was concentrated in vacuum to give crude product. The crude product was purified by reverse phase HPLC to afford Compound 149 (4 mg, 1% yield) as a yellow solid.

LCMS: (ES, m/z): [M+H]⁺ 406

H-NMR: (400 MHz, DMSO-d₆, ppm): δ 8.42 (s, 1H), 7.90-7.73 (m, 3H), 7.51-7.37 (m, 3H), 7.12 (d, J=6.4 Hz, 1H), 6.30 (t, J=6.8 Hz, 1H), 4.55 (s, 2H), 3.69 (s, 3H)

### Example 150. Synthesis of Compound 150

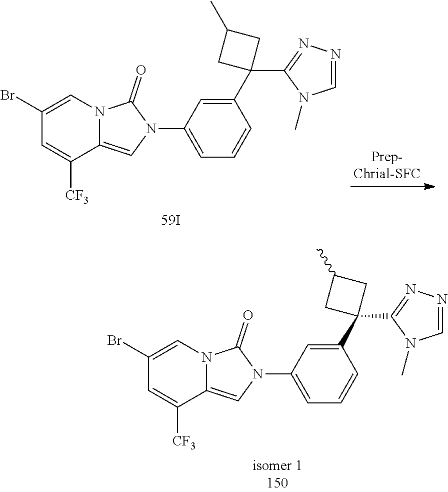

### Synthesis of 150

59i (150 mg) was purified by Prep-SFC with the following conditions (Column: CHIRALPAK IG, 3*25 cm, 5 μm; Mobile Phase A: CO, Mobile Phase B: MeOH (0.1% 2M NH₃-MeOH); Flow rate: 70 mL/min; Gradient: isocratic 50% B; Column Temperature (° C.): 35; Back Pressure (bar): 100; Wave Length: 220 nm; RT1 (min): 9.31, RT2 (min): 13.5, the second peak is product) to Compound 150 (624 ng, 15.52%) as a yellow solid.

LCMS: (ES, m/z): [M+H]⁺ 506.

H-NMR: (400 MHz, DMSO-d₆, ppm): δ 1.09-1.11 (d, 3H), 2.22-2.29 (m, 2H), 2.31-2.36 (m, 1H), 3.10-3.14 (m, 2H), 3.24 (s, 3H), 7.09-7.11 (d, 1H), 7.16 (s, 1H), 7.45-7.49 (m, 2H), 7.60-7.62 (m, 1H), 7.69 (s, 1H), 8.03 (s, 1H), 8.36 (s, 1H).

### Example 151. Synthesis of Compound 151

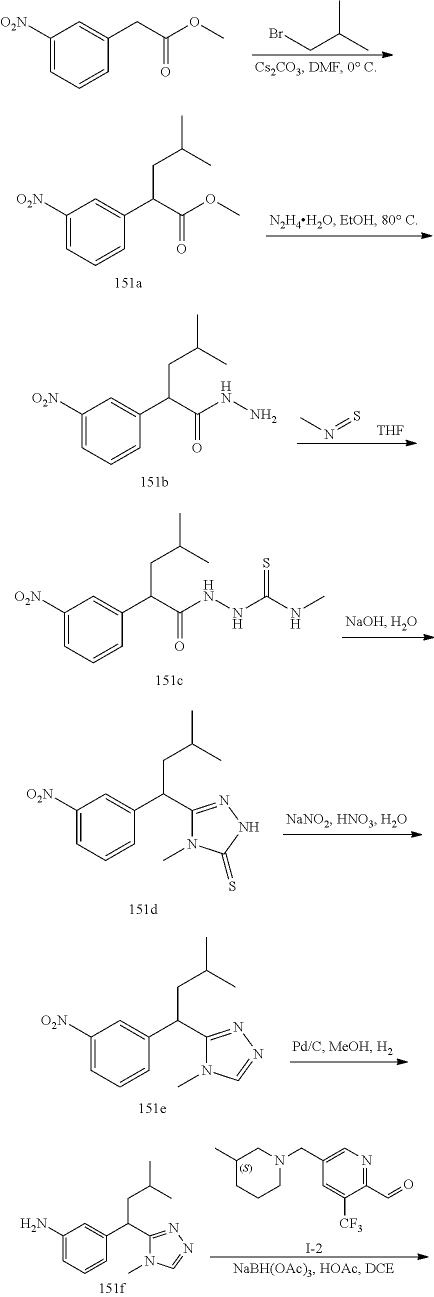

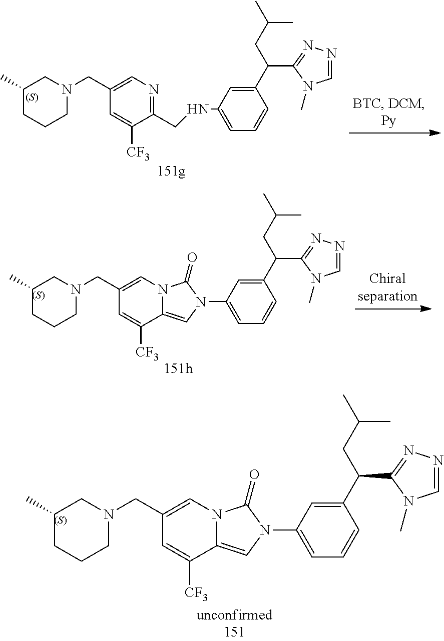

### Synthesis of 151a

To a stirred solution of methyl 2-(3-nitrophenyl)acetate (20 g, 102.473 mmol, 1 equiv) in DMF (500 mL) was added Cs₂CO₃ (100.16 g, 307.419 mmol, 3 equiv) at 0° C. under nitrogen atmosphere. The resulting mixture was stirred for 3h at 0° C. To the above mixture was added isobutyl bromide (42.12 g, 307.419 mmol, 3 equiv) dropwise at 0° C. The resulting mixture was stirred overnight at room temperature. The resulting mixture was diluted with water (1.5 L). The aqueous layer was extracted with EtOAc (2×800 mL). The residue was purified by silica gel column chromatography, eluted with PE/EA (10:1) to afford 151a (12.5 g, 45.15%) as an off-white solid.

### Synthesis of 151b

To a stirred solution of 151a (12 g, 47.755 mmol, 1 equiv) in EtOH (100 mL) was added hydrazine hydrate (98%) (23.91 g, 477.550 mmol, 10 equiv) at room temperature. The resulting mixture was stirred overnight at 80° C. The resulting mixture was diluted with water (500 mL). The aqueous layer was extracted with DCM/MeOH (10/1) (5×300 mL). The resulting mixture was dried with N2SO₄, filtered, and the filter cake was washed with MeOH (3×100 mL). The filtrate was concentrated under reduced pressure to afford 151b (9 g, 69.00%) as a yellow solid.

### Synthesis of 151c

To a stirred solution of 151b (9 g, 35.816 mmol, 1 equiv) in tetrahydrofuran (100 mL) was added methyl isothiocyanate (5.24 g, 71.63 mmol, 2 equiv) at room temperature. The resulting mixture was stirred for 4 h at room temperature. The resulting mixture was diluted with water (300 mL). The precipitated solids were collected by filtration and washed with water (2×50 mL) to afford 151c (10 g, 79.18%) as a yellow solid.

### Synthesis of 151d

To a stirred solution of NaOH (9.86 g, 246.608 mmol, 8 equiv) in H₂O (250 mL) was added 151 (10 g, 30.826 mmol, 1 equiv) at room temperature. The resulting mixture was stirred for 2 h at room temperature. The resulting mixture was diluted with water (500 mL). The mixture was acidified to pH 5 with HCl (1 M). The aqueous layer was extracted with DCM/MeOH (10/1) (3×500 mL). The resulting mixture was concentrated under reduced pressure to afford 151d (9 g, 85.76%) as a yellow solid.

### Synthesis of 151e

To a stirred solution of 151d (9 g, 29.375 mmol, 1 equiv) and NaNO₂ (20.27 g, 293.750 mmol, 10 equiv) in 1H₂O (100 mL) and EA (200 mL) was added HNO₃ (293.75 mL, 293.750 mmol, 10 equiv) dropwise at 0 degrees C. The resulting mixture was stirred overnight at room temperature. The reaction was quenched by the addition of NaHCO₃ (aq.)(1 L) at room temperature. The aqueous layer was extracted with DCM/MeOH (10/1)(3×1 L). The resulting mixture was concentrated under reduced pressure to afford 151e (6.8 g, 78.48%) as a yellow solid.

### Synthesis of 151f

To a solution of 151e (6.8 g, 24.788 mmol, 1 equiv) in MeOH (200 mL) was added Pd/C (1 g, 9.397 mmol, 0.38 equiv) under nitrogen atmosphere in a 1000 mL round-bottom flask. The mixture was hydrogenated at room temperature overnight under hydrogen atmosphere using a hydrogen balloon, filtered through a Celite pad, and concentrated under reduced pressure. This resulted in 151f (4 g, 59.44%) as a yellow solid.

### Synthesis of 151g

To a stirred solution of 151f (500 mg, 2.046 mmol, 1.00 equiv) and I-2 (585.86 mg, 2,046 mmol, 1.00 equiv) in DCE (8 mL) was added HOAc (122.89 mg, 2.046 mmol, 1 equiv) and NaBH(OAc)3 (867.39 mg, 4,092 mmol, 2 equiv) at room temperature under nitrogen atmosphere. The resulting mixture was stirred for 3h at room temperature. The reaction was quenched with saturated aqueous NH₄Cl at room temperature. The aqueous layer was extracted with DCM (2×50 mL). The organic layers were concentrated under reduced pressure. The residue was purified by Prep-TLC (DCM/MeOH 12:1) to afford 151g (500 mg, 43.21%) as a light yellow oil.

### Synthesis of 151h

To a stirred solution of 1 Sig (500 mg, 0.972 mmol, 1 equiv) and Pyridine (768.50 mg, 9.720 mmol, 10 equiv) in DCM (10 mL) was added Triphosgene (100.91 mg, 0.340 mmol, 0.35 equiv) at room temperature. The resulting mixture was stirred for 10 min at room temperature. The reaction was quenched with NaHCO₃ (aq.) at room temperature. The aqueous layer was extracted with DCM (2×20 mL). The residue was purified by Prep-TLC (DCM/MeOH 12:1) to afford 151h (280 mg, 52.24%) as a yellow solid.

### Synthesis of 151

The 151h (280 mg) was purified by chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 13.5 min; Wave Length: 220/254 nm; RT1 (min): 9.65; RT2 (min): 11.67, the first peak is product) to afford Compound 151 (98.9 mg, 35.00%) as a yellow solid.

LC-MS: (ES, m/z): [M+H]⁺ 541

H-NMR: (400 MHz, DMSO-d₆, 3 ppm): 0.79-0.83 (m, 4H), 0.83-0.90 (d, 3H), 0.90-0.96 (d, 3H), 1.44-1.51 (m, 2H), 1.55-1.66 (m, 4H), 1.85-1.94 (m, 2H), 2.10-2.17 (m, 1H), 2.72-2.77 (m, 2H), 3.24 (s, 2H), 3.46 (s, 3H), 4.29-4.33 (m, 1H), 7.00 (s, 1H), 7.22-7.24 (d, 1H), 7.30 (s, 1H), 7.43-7.47 (m, 1H), 7.68-7.70 (m, 2H), 7.77 (d, 1H), 8.33 (s, 1H).

### Example 152. Synthesis of Compound 152

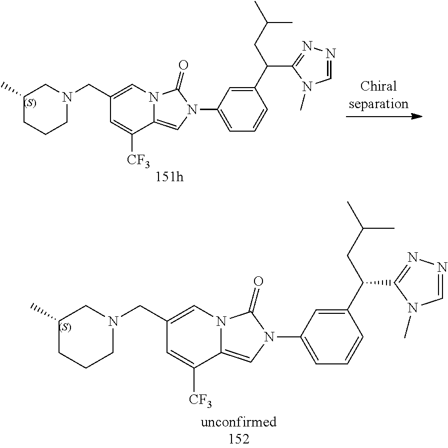

### Synthesis of 152

The 151h (280 mg) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 40% B in 13.5 min; Wave Length: 220/254 nm; RT1 (min): 9.65; RT2 (min): 11.67, the second peak is product) to afford Compound 152 (95.9 mg, 32.85%) as a yellow solid.

LC-MS: (ES, m/z): [M+H]⁺ 541

H-NMR: (400 MHz, DMSO-d₆, 3 ppm): 0.81-0.83 (m, 4H), 0.83-0.89 (d, 3H), 0.89-0.94 (d, 3H), 1.43-1.49 (m, 2H), 1.55-1.66 (m, 4H), 1.87-1.94 (m, 2H), 2.10-2.15 (m, 1H), 2.72-2.76 (m, 2H), 3.24 (s, 2H), 3.46 (s, 3H), 4.29-4.33 (m, 1H), 7.00 (s, 1H), 7.22-7.24 (d, 1H), 7.30 (s, 1H), 7.43-7.47 (m, 1H), 7.68-7.70 (m, 2H), 7.77 (d, 1H), 8.33 (s, 1H).

### Example 153. Synthesis of Compound 153

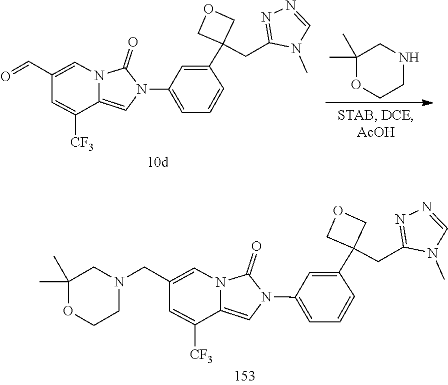

### Synthesis of 153

To a stirred solution of 10d (300 mg, 0.656 mmol, 1 equiv) and 2,2-dimethylmorpholine (151.08 mg, 1.312 mmol, 2 equiv) in DCE (5 mL) were added STAB (278.01 mg, 1312 mmol, 2 equiv) and AcOH (39.39 mg, 0.656 mmol, 1 equiv) at room temperature. The resulting mixture was stirred overnight at room temperature. The reaction was quenched by the addition of sat. NH₄Cl (aq.) (8 mL) at room temperature. The resulting mixture was extracted with EtOAc (3×10 mL), dried over anhydrous Na₂SO₄. After filtration, the filtrate was concentrated under reduced pressure. The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 8 min; Wave Length: 220 nm; RT1 (min): 7.17) to afford Compound 153 (119.5 mg, 32.74%) as a yellow solid.

LC-MS: (ES, m/z): [M+H]⁺:557

H-NMR: (400 MHz, DMSO-d6, ppm): δ 1.14 (s, 6H), δ 2.16 (s, 2H), δ 2.33 (s, 2H), δ 2.97 (s, 3H), δ 3.26 (s, 2H), δ 3.53 (s, 2H), δ 3.60-3.63 (m, 2H), δ 4.91-4.92 (d, 2H), δ 4.94-4.96 (d, 2H), δ 6.88-6.90 (d, 1H), δ 7.04 (s, 1H), δ 7.31 (s, 1H), δ 7.38-7.42 (m, 2H), δ 7.68 (s, 1H), S 7.74-7.76 (m, 1H), δ 8.20 (s, 1H).

### Example 154. Synthesis of Compound 154

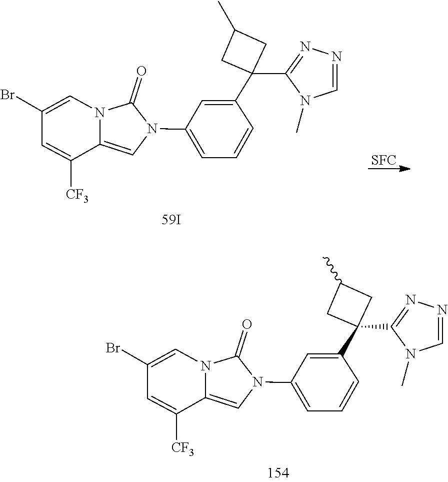

### Synthesis of 154

59i (150 mg) was purified by Prep-Chiral-SFC with the following conditions (Column: CHIRALPAK IG, 3*25 cm, 5 um; Mobile Phase A: C₇, Mobile Phase B: MeOH (0.1% 2M NH₃-MeOH); Flow rate: 70 mL/min; Gradient: isocratic 50% B; Column Temperature (° C.): 35; Back Pressure (bar): 100; Wave Length: 220 m; RT1 (min): 9.31, RT2 (min): 13.5, the first peak is product) to Compound 154 (247.8 mg, 61.63%) as a yellow solid,

LCMS: (ES, m/z): [M+H]⁺ 506.

H-NMR: (400 MHz, DMSO-d₆, ppm): δ 1.08-1.09 (d, 3H), 2.54-2.57 (m, 3H), 2.86-2.90 (m, 2H), 3.20 (s, 3H), 7.16 (s, 1H), 7.25-7.27 (d, 1H), 7.48-7.52 (m, 2H), 7.63-7.65 (d, 1H), 7.83 (s, 1H), 8.03 (s, 1H), 8.29 (s, 1H).

### Example 155. Synthesis of Compound 155

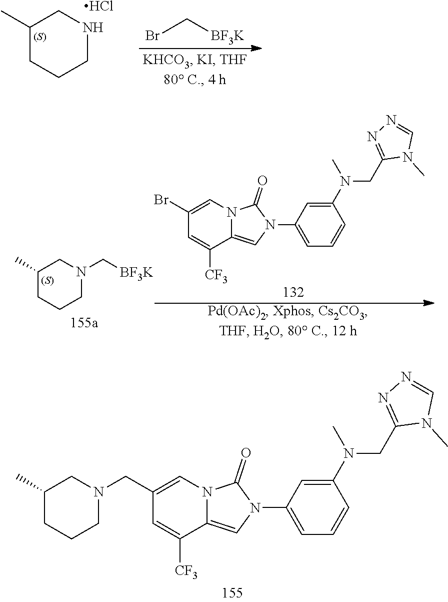

### Synthesis of 155a

A mixture of (3S)-3-methylpiperidine (1 g, 7.37 mmol), bromomethyl(trifluoro)boron;potassium (1.48 g, 7.37 mmol), KHCO₃ (1.48 g, 14.75 mmol) KI (122.39 mg, 737.28 umol) in THE (10 mL) was degassed and purged with N₂ for 3 times, and then the mixture was stirred at 80° C. for 4 hr under N₂. The reaction mixture was concentrated under reduced pressure to give a residue. The residue dissolved in acetone (10 ml), filtered, and the filtrate was concentrated under reduced pressure to give a residue. The crude product 155a (800 mg, 80% purity) was used into the next step without further purification.

Alternatively, 155a can be prepared as outlined below:
To a solution of (3S)-3-methylpiperidine (1.0 g, 7.37 mmol, as a hydrochloride salt), potassium;bromomethyl(trifluoro)boranuide (1.48 g, 7.37 mmol), potassium bicarbonate (1.48 g, 14.7 mmol), KI (122 mg, 737 umol) in THF (20 mL) was stirred at 90° C. for 12 h under N₂. The mixture was concentrated under reduced pressure. The crude product was triturated with acetone (30 ml) at 25° C. for 30 min then filtered. The filtrate was concentrated under reduced pressure to afford 155a (1.2 g, 74% yield) as a light yellow oil.

### Synthesis of 155

A mixture of 132 (100 mg, 207.78 umol), 155a (136.57 mg, 623.34 umol), Pd(OAc)2 (4.66 mg, 20.78 umol), XPhos (19.81 mg, 41.56 umol) and Cs₂CO₃ (203.10 mg, 623.34 umol) in THF (4 mL) and H₂O (1 mL) was degassed and purged with N₂ for 3 times, and then the mixture was stirred at 80° C. for 12 hr under N2 atmosphere. The reaction mixture was quenched by addition of saturated aqueous sodium carbonate solution (10 mL) and extracted with DCM (10 mL*2). The combined organic layers were dried over anhydrous sodium sulfate, filtered, and the filtrate was concentrated under reduced pressure to give a residue. The residue was purified by reverse phase HPLC (column: Phenomenex C18 75*30 mm*3m; mobile phase: [water (NH₃H₂O+NH₄CO₃)-ACN]; B %: 45%-75%, 8 min) to afford Compound 155 (9 mg, 8.43% yield) as a yellow solid.

LCMS: (ES, m/z): [M+H]⁺ 514.

H-NMR: (400 MHz, DMSO-d₆, ppm): δ 8.40 (s, 1H), 7.657 (s, 1H), 7.28 (d, J=6 Hz, 2H), 7.22 (s, 1H), 7.13 (d, J=7.6 Hz, 1H), 6.99 (s, 1H), 6.89-6.87 (m, 1H), 4.754 (s, 2H), 3.62 (s, 3H), 3.24 (s, 2H), 2.99 (s, 3H), 2.77-2.72 (m, 2H), 1.92-1.89 (m, 1H), 1.66-1.58 (m, 4H), 146-1.43 (m, 1H), 0.83-0.82 (m, 3H)

### Example 156. Synthesis of Compound 156

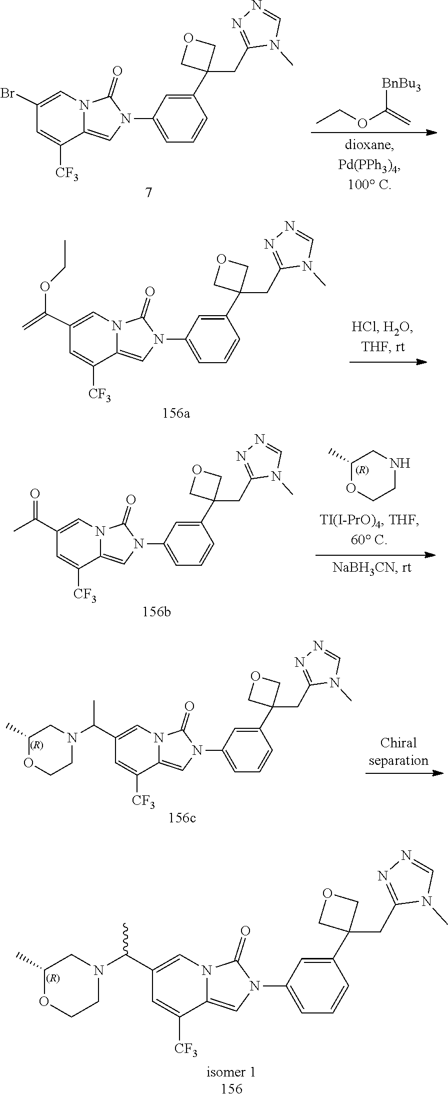

### Synthesis of 156a

To a stirred mixture of 7 (4 g, 7.869 mmol, 1 equiv) and tributyl (1-ethoxyethenyl) stannane (4.26 g, 1.803 mmol, 1.5 equiv) in dioxane (40 ml) was added Pd(PPh₃)₄ (0.91 g, 0.787 mmol, 0.1 equiv) at room temperature under nitrogen atmosphere. The resulting mixture was stirred overnight at 100° C. under nitrogen atmosphere. The resulting mixture was diluted with water (50 mL). The aqueous layer was extracted with CH₂Cl₂/MeOH (10:1) (3×100 mL). The resulting mixture was concentrated under reduced pressure. The residue was purified by trituration with tert-Butyl methyl ether (50 mL). The precipitated solids were collected by filtration and washed with tert-Butyl methyl ether (3×20 mL). The resulting mixture was concentrated under reduced pressure to afford 156a (3.2 g, 74.90%) as a yellow solid.

### Synthesis of 156b

To a stirred mixture of 156a (3.2 g, 6,406 mol, 1 equiv) in THE (30 mL) was added HCl (33 mL, 1 M) at room temperature. The resulting mixture was stirred overnight at room temperature. The resulting mixture was diluted with water (50 mL). The aqueous layer was extracted with CH₂Cl₂/MeOH (10:1) (3×100 mL). The resulting mixture was concentrated under reduced pressure to afford 156b (2.4 g, 71.52%) as a yellow solid.

### Synthesis of 156c

To a stirred mixture of 156b (2.4 g, 5.091 mmol, 1 equiv) and (2R)-2-methylmorpholine (1.54 g, 15.273 mmol, 3 equiv) in THF (25 mL) was added Ti(Oi-Pr)₄ (5.79 g, 20.364 mmol, 4 equiv) at room temperature. The resulting mixture was stirred for 3h at 60° C. To the above mixture was added NaBH₃CN (0.96 g, 15.273 mmol, 3 equiv) at room temperature. The resulting mixture was stirred overnight at room temperature. The reaction was quenched by the addition of saturated aqueous NH₄Cl (60 mL) at room temperature. The aqueous layer was extracted with CH₂Cl₂/MeOH (10:1) (3×50 mL). The resulting mixture was concentrated under reduced pressure. The residue was purified by silica gel column chromatography, eluted with CH₂Cl₂/MeOH (60:1) to afford 156c (800 mg). The residue was purified by Prep-TLC (CH₂Cl₂/MeOH 12:1) to afford 156c (600 mg). The residue was purified by reverse flash chromatography with the following conditions: column, silica gel; Mobile Phase A: Water (10 mmol/L NH₄HCO₃), Mobile Phase B: ACN, 10% to 90% gradient in 40 min; detector, LV 254 nm to afford 156c (400 mg 13.83%) as a yellow solid.

### Synthesis of 156

156c (400 mg) was purified by chiral separation with the following conditions (Column: CHIRALPAK IC, 2*25 cm, 5 μm; Mobile Phase A: Hex (0.5% 2M NH3-MeOH), Mobile Phase B: MeOH:DCM=1:1; Flow rate: 20 mL/min; Gradient: 65% B to 65% B in 14 min; Wave Length: 220/254 min; RT1 (min): 10.91; RT2 (min): 12.43; the first peak is product) to afford 156 (130 mg). The residue was purified by Prep-TLC (CH₂Cl₂/MeOH 12:1) to afford 156 (70 mg). The crude product (70 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 50% B in 8 min, Wave Length: 220 nm; RT1 (min): 7.82) to afford Compound 156 (54 mg, 13.12%) as a yellow solid.

LCMS: (ES, m/z): [M+H]⁺ 557

H-NMR: H NMR (400 MHz, DMSO, ppm): δ 0.92-1.10 (d, 3H), δ 1.24-1.26 (d, 3H), δ 1.74-1.79 (m, 1H), δ 2.03-2.08 (m, 1H), δ 2.67-2.74 (m, 2H), δ 2.97 (s, 3H), δ 3.44-3.54 (m, 5H), δ 3.76-3.79 (d, 1H), δ 4.90-4.97 (m, 4H), δ 6.98-6.92 (d, 1H), δ 7.05 (s, 1H), δ 7.32 (s, 1H), δ 7.38-7.43 (m, 2H), δ 7.62 (s, 1H), δ 7.73-7.77 (m, 1H), δ 8.20 (s, 1H).

### Example 157. Synthesis of Compound 157

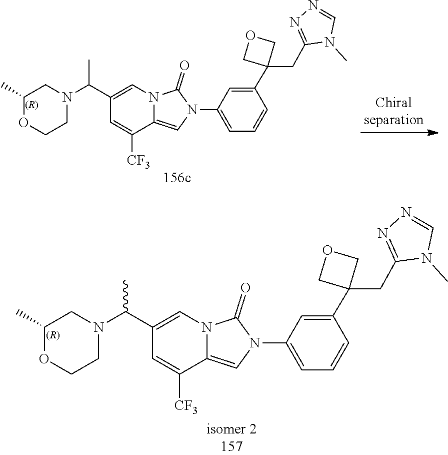

### Synthesis of 157

156c (400 mg) was purified by chiral separation with the following conditions (Column: CHIRALPAK IC, 2*25 cm, 5 μm; Mobile Phase A: Hex (0.5% 2M NH3-MeOH), Mobile Phase B: MeOH:DCM=1:1; Flow rate: 20 mL/min; Gradient: 65% B to 65% B in 14 min; Wave Length: 220/254 nm; RT1 (min): 10.91; RT2 (min): 12.43; the second peak is product) to afford Compound 157 (120.9 mg, 29.08%) as a yellow solid.

LCMS: (ES, m/z): [M+H]⁺ 557

H-NMR: H NMR (400 MHz, DMSO, ppm): δ 1.10 (s, 3H), δ 1.24-1.26 (s, 3H), δ 1.79 (s, 1H), δ 2.10 (s, 1H), δ 2.61-2.68 (m, 1H), δ 2.79-2.89 (m, 1H), δ 2.97 (s, 3H), δ 3.33-3.54 (m, 5H), δ 3.64-3.75 (m, 1H), δ 4.81-5.00 (m, 4H), δ 6.98-6.92 (d, 1H), δ 7.06 (s, 1H), δ 7.33 (s, 1H), δ 7.39-7.43 (m, 2H), δ 7.64 (s, 1H), δ 7.74-7.77 (m, 1H), δ 8.20 (s, 1H).

### Example 158. Synthesis of Compound 158

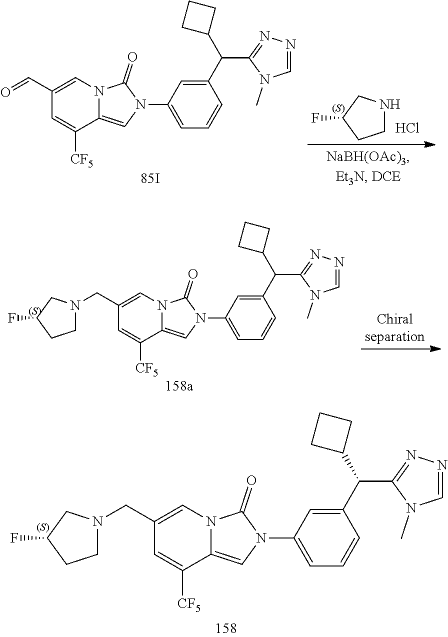

### Synthesis of 158a

To a stirred solution of 85i (800 mg, 1.757 mmol, 1 equiv) and (3S)-3-fluoropyrrolidine hydrochloride (441.14 mg, 3.514 mmol, 2 equiv) in DCE (10 mL) were added Et₃N (355.49 mg, 3.514 mmol, 2 equiv) at room temperature under nitrogen atmosphere. The resulting mixture was stirred for 1h at room temperature. To the above mixture was added NaBH(OAc)3 (744.57 mg, 3.514 mmol, 2 equiv) at room temperature. The resulting mixture was stirred for additional 3h at room temperature. The reaction was quenched with NH₄Cl (aq.) at room temperature. The aqueous layer was extracted with DCM (2×20 mL). The resulting mixture was concentrated under reduced pressure. The residue was purified by Prep-TLC (DCM/MeOH 18:1) to afford 158a (380 mg, 39.70%) as a yellow solid.

### Synthesis of 158

The 158a (380 mg, 0,719 mmol, 1 equiv) was purified by chiral separation with the following conditions (Column: CHIRALPAK IC, 2*25 cm, 5 μm; Mobile Phase A: Hex (0.5% 2M NH₃-MeOH), Mobile Phase B: MeOH:DCM=1:1 (01% 2M NH₃-MeOH); Flow rate: 20 mL/min; Gradient: 45% B to 45% B in 13 min; Wave Length: 2201/254 nm; RT1 (min): 9.30; RT2 (min): 11.45; the first peak is product) to afford Compound 158 (129.8 mg, 33.30%) as a yellow solid.

LC-MS: (ES, m/z): [M+H]⁺ 529

H-NMR: (400 MHz, DMSO-d₆, 3 ppm): 1.68-1.95 (m, 6H), 2.01-2.21 (m, 2H), 2.31-2.43 (m, 1H), 2.61-2.71 (m, 1H), 2.81-2.91 (m, 2H), 3.15-3.29 (m, 1H), 3.34-3.47 (m, 5H), 4.25-4.27 (d, 2H), 5.14-5.28 (m, 1H), 6.94-6.96 (d, 1H), 7.03 (s, 1H), 7.18-7.20 (m, 1H), 7.32 (s, 1H), 7.42-7.46 (m, 1H), 7.68-7.74 (m, 3H), 8.33 (s, 1H).

### Example 159. Synthesis of Compound 159

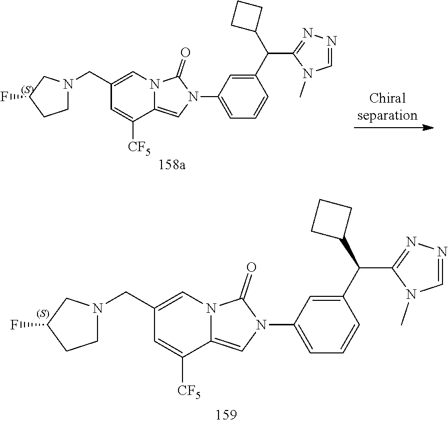

### Synthesis of 159

The 158a (380 mg, 0.719 mmol, 1 equiv) was purified by chiral separation with the following conditions (Column: CHIRALPAK IC, 2*25 cm, 5 μm; Mobile Phase A: Hex (0.5% 2M NH₃-MeOH), Mobile Phase B: MeOH:DCM=1:1 (0.1% 2M NH₃-MeOH); Flow rate: 20 mL/min; Gradient: 45% B to 45% B in 13 min; Wave Length: 220/254 nm; RT1 (min): 9.30; RT2 (min): 11.45; the second peak is product) to afford Compound 159 (134.1 mg, 35.29%) as a yellow solid.

LC-MS: (ES, m/z): [M+H]⁺ 529

H-NMR: (400 MHz, DMSO-d₆, 3 ppm): 1.68-1.95 (m, 6H), 2.01-2.21 (m, 2H), 2.31-2.43 (m, 1H), 2.61-2.71 (m, 1H), 2.81-2.91 (m, 2H), 3.15-3.29 (m, 1H), 3.34-3.47 (m, 5H), 4.25-4.27 (d, 2H), 5.14-5.28 (m, 1H), 6.94-6.96 (d, 1H), 7.03 (s, 1H), 7.18-7.20 (m, 1H), 7.32 (s, 1H), 7.42-7.46 (m, 1H), 7.68-7.74 (m, 3H), 8.33 (s, 1H).

### Example 160. Synthesis of Compound 160

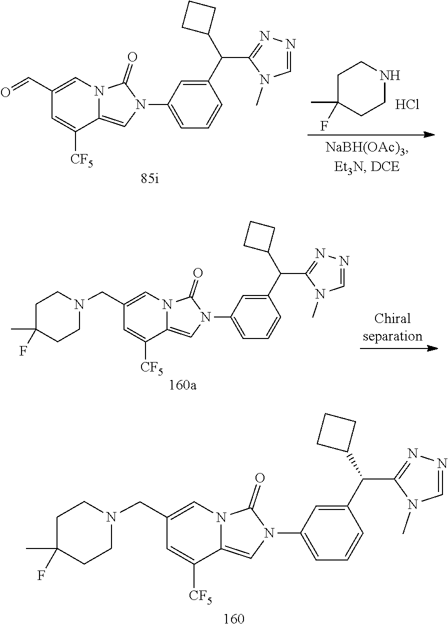

### Synthesis of 160a

To a stirred mixture of 4-fluoro-4-methylpiperidine hydrochloride (269.86 mg, 1.757 mmol, 1 equiv) and 85i (800 mg, 1.757 mmol, 100 equiv) in DCE (8 mL) was added Et₃N (533.23 mg, 5.271 mmol, 3 equiv) at room temperature. The resulting mixture was stirred for 30 min at room temperature. To the above mixture was added NaBH(OAc)3 (1116.85 mg, 5.271 mmol, 3 equiv) at room temperature. The resulting mixture was stirred overnight at room temperature. The reaction was quenched by the addition of H₂O (30 mL) at room temperature. The aqueous layer was extracted with CH₂Cl₂ (2×10 mL). The organic layers were concentrated under reduced pressure. The residue was purified by Prep-TLC (CH₂Cl₂/MeOH=15:1) to afford 160a (400 mg) as a yellow solid. The crude product (400 mg) was purified by Prep-HPLC with the following conditions (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 40% B to 65% B in 8 min, Wave Length: 220 nm; RT1 (min): 6.90) to afford 160a (200 mg, 19.84%) as a yellow solid.

### Synthesis of 160

160a (200 mg) was purified by chiral separation separation with the following condition: (Column: CHIRALPAK. IC, 2*25 cm, 5 μm; Mobile Phase A: Hex (0.5% 2M NH₃-MeOH), Mobile Phase B: EtOH:DCM=1:1; Flow rate: 20 mL/min; Gradient: 50% B to 50% B in 18 min; Wave Length: 220/254 nm; RT1 (min): 8.47, RT2 (min): 14.52, the first peak is product) to afford Compound 160 (73.6 mg, 36.73%) as a yellow solid.

LCMS: (ES, m/z): [M+H]⁺ 557.

H-NMR: (400 MHz, DMSO-d₆, ppm): δ 1.30-1.42 (d, 3H), 1.58-1.85 (m, 9H), 2.02-2.09 (m, 1H), 2.24-2.29 (m, 2H), 2.60-2.63 (m, 2H), 3.19-3.23 (m, 1H), 3.25 (s, 2H), 3.43 (s, 3H), 4.25-4.27 (d, 1H), 7.01 (s, 1H), 7.18-7.20 (d, 1H), 7.31 (s, 1H), 7.42-7.46 (m, 1H), 7.68-7.73 (m, 3H), 8.33 (s, 1H).

### Example 161. Synthesis of Compound 161

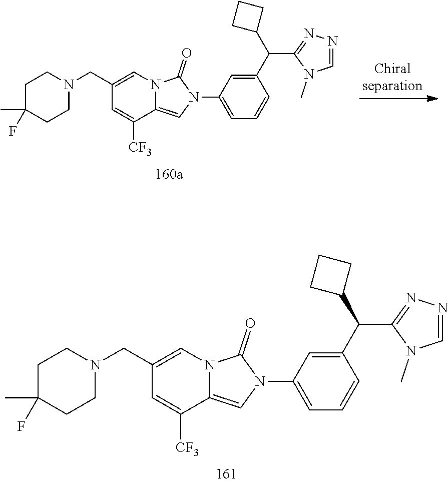 [SMILES: Cn1cnnc1C(c1cccc(-n2cc3c(C(F)(F)F)cc(CN4CCC(C)(F)CC4)cn3c2=O)c1)C1CCC1 ; Cn1cnnc1[C@@H](c1cccc(-n2cc3c(C(F)(F)F)cc(CN4CCC(C)(F)CC4)cn3c2=O)c1)C1CCC1]

### Synthesis of 161

160a (200 mg) was purified by chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 50% B to 50% B in 18 min; Wave Length: 220/254 nm; RT1 (min): 8.47, RT2 (min): 14-52, the second peak is product) to afford Compound 161 (54.9 mg, 27.34%) as a yellow solid.

LCMS: (ES, m/z): [M+H]⁺ 557.

H-NMR: (400 MHz, DMSO-d₆, ppm): δ 1.30-1.42 (d, 3H), 1.61-1.81 (m, 9H), 2.02-2.09 (m, 1H), 2.27-2.34 (m, 2H), 2.60-2.63 (m, 2H), 3.18-3.23 (m, 1H), 3.25 (s, 2H), 3.43 (s, 3H), 4.25-4.27 (d, 1H), 7.01 (s, 1H), 7.18-7.20 (d, 1H), 7.31 (s, 1H), 7.42-7.46 (m, 1H), 7.68-7.73 (m, 3H), 8.33 (s, 1H).

Alternatively, Compound 161 may be also prepared in the manner outlined below:

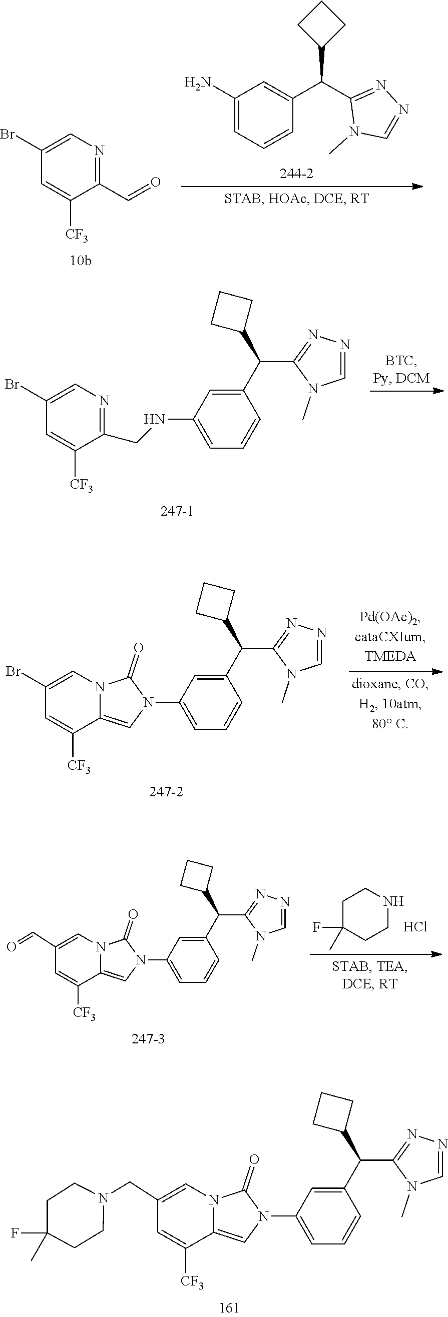

### 1. Synthesis of 247-1

To a stirred solution of 10b (72.95 g, 287.216 mmol, 1.2 equiv) and 244-2 (58 g, 239.347 mmol, 1.00 equiv) in DCE (800 mL) were added STAB (101.45 g, 478.694 mmol, 2 equiv) and HOAc (7.19 g, 119.674 mmol, 0.5 equiv) at room temperature. The resulting mixture was stirred overnight at room temperature. The reaction was quenched with saturated NaHCO3 (aq.) (800 mL) at room temperature. The resulting mixture was extracted with DCM (3×400 mL). After filtration, the filtrate was concentrated under reduced pressure. The residue was purified by silica gel column chromatography, eluted with DCM/MeOH (50:1) to afford 247-1 (90 g, 74.37%) as a light yellow solid.

### 2, Synthesis of 247-2

To a stirred solution of 247-1 (90 g, 187.370 mmol, 1 equiv) and Pyridine (88.93 g, 1124.274 mmol, 6.00 equiv) in DCM (1000 ml) were added triphosgene (19.46 g, 65.582 mmol, 0.35 equiv) at 0° C. under nitrogen atmosphere. The resulting mixture was stirred for 20 min at room temperature under nitrogen atmosphere. The reaction was quenched with saturated NaHCO₃ (aq.) (800 mL) at room temperature. The resulting mixture was extracted with DCM (3×300 mL). The resulting mixture was concentrated under vacuum. The residue was purified by trituration with MTBE (300 mL), This resulted in 247-1 (80 g, 80.11%) as a yellow solid.

### 3. Synthesis of 247-3

To a stirred solution of 247-2 (40 g, 79,000 mmol, 1 equiv) and TMEDA (18.36 g, 158.000 mmol, 2 equiv) in 1,4-dioxane (1.2 L) were added Pd(OAc)2 (1.77 g, 7,884 mmol, 0.10 equiv) and bis(adamantan-1-yl)(butyl)phosphane (5.66 g, 15.786 mmol, 0.20 equiv) at room temperature. The mixture was purged with nitrogen for 3 mm and then was pressurized to 10 atm with CO:H2=1:1 at 80° C. for overnight. The reaction mixture was cooled to room temperature and concentrated under reduced pressure. The residue was purified by silica gel column chromatography, eluted with DCM/MeOH (30:1) to afford 247-3 (24 g, 63.37%) as a yellow solid,

### 4. Synthesis of 161

To a stirred solution of 247-3 (76 g, 166.871 mmol, 1.00 equiv) and 4-fluoro-4-methylpiperidine hydrochloride (38.45 g, 250,307 mmol, 1.5 equiv) in DCE (1000 m-L) were added TEA (33.77 g, 333.742 mmol, 2 equiv) at room temperature. The resulting mixture was stirred for 1h at room temperature. To the above mixture was added STAB (70.73 g, 333.742 mmol, 2 equiv) at room temperature. The resulting mixture was stirred for 4h at room temperature. The reaction was quenched with NH4Cl (aq.) (1500 mL) at room temperature. The resulting mixture was extracted with CH2Cl2 (3×1500 mL). The resulting mixture was concentrated under reduced pressure. The residue was purified by reverse flash chromatography with the following conditions: column, C18 silica gel; mobile phase, MeCN in Water (10 mmol/L NH4HCO3), 30% to 55% gradient in 20 min; detector, UV 254 nm. This resulted in 161 (28.0756 g, 29.77%) as a yellow solid.

LCMS-161: (ES, m/z): [M+H]⁺ 557. H-NMR-161: (400 MHz, DMSO-d6, δ ppm): 1.30-1.42 (d, 3H), 1.61-1.81 (m, 9H), 2.02-2.18 (m, 1H), 2.23-2.34 (m, 2H), 2.59-2.65 (m, 2H), 3.18-3.23 (m, 1H), 3.25 (s, 2H), 3.43 (s, 3H), 4.25-4.27 (d, 1H), 7.01 (s, 1H), 7.18-7.20 (d, 1H), 7.30 (s, 1H), 7.42-7.46 (m, 1H), 7.67-7.74 (m, 3H), 8.33 (s, 1H).

### Example 162. Synthesis of Compound 162

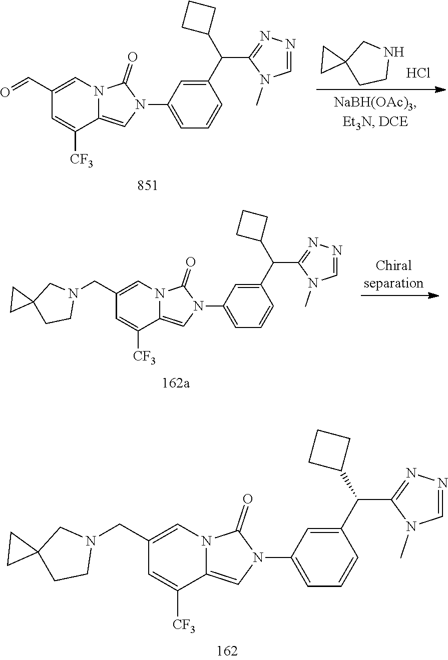

### Synthesis of 162a

To a stirred solution of 5-azaspiro[2.4]heptane hydrochloride (352.06 mg, 2.635 mmol, 15 equiv) and 85i (800 mg, 1,757 mmol, 1.00 equiv) in DCE (8 ml) was added Et₃N (533.23 mg, 5.271 mmol, 3 equiv) at room temperature. The resulting mixture was stirred for 30 min at 0° C. To the above mixture was added NaBH(OAc)₃ (1116.85 ng, 5.271 mmol, 3 equiv) at room temperature. The resulting mixture was stirred overnight at room temperature. The reaction was quenched by the addition of 30 ml, H₂O at room temperature. The aqueous layer was extracted with CH₂Cl₂ (2×10 mL). The organic layers were concentrated under reduced pressure. The residue was purified by Prep-TLC (CH₂Cl₂/MeOH=15:1) to afford 162a (400 mg) as a yellow solid, 162a (400 mg) was purified by Prep-HPLC with the following conditions (Column: YMC-Actus Triart C18 ExRS, 30*150 mm, 5 μm; Mobile Phase A: water (10 mmol/L NH₄HCO₃), Mobile Phase B: ACN; Flow rate: 60 mL/min; Gradient: 45% B to 65% B in 8 min, Wave Length: 220 am; RT1 (min): 7.15) to afford 162a (240 mg, 24.95%) as a yellow solid.

### Synthesis of 162

162a (240 mg) was purified by chiral separation separation with the following conditions (Column: CHIRALPAK IC, 2*25 cm, 5 μm; Mobile Phase A: Hex (0.5% 2 M NH₃-MeOH), Mobile Phase B: EtOH:DCM=1:1; Flow rate: 20 mL/min; Gradient: 50% B to 50% B in 13 min; Wave Length: 220/254 nm; RT1 (min): 7.75, RT2 (min): 10.35, the first peak is product) to afford Compound 162 (109.2 mg, 45.27%) as a yellow solid.

LCMS: (ES, m/z): [M+H]⁺ 537.

H-NMR: (400 MHz, DMSO-d₆, ppm): δ 0.51-0.53 (d, 4H), 1.70-1.80 (m, 7H), 2.07-2.09 (m, 1H), 2.45-2.50 (m, 2H), 2.66-2.69 (m, 2H), 3.13-3.25 (m, 1H), 3.39 (s, 2H), 3.43 (s, 3H), 4.25-4.27 (d, 1H), 7.04 (s, 1H), 7.18-7.20 (d, 1H), 7.31 (s, 1H), 7.42-7.46 (m, 1H), 7.67-7.73 (m, 3H), 8.32 (s, 1H).

### Example 163. Synthesis of Compound 163

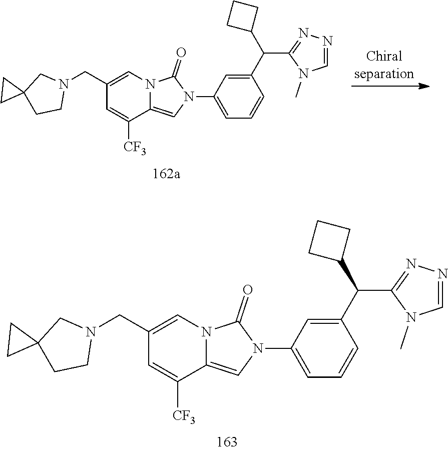 [SMILES: Cn1cnnc1C(c1cccc(-n2cc3c(C(F)(F)F)cc(CN4CCC5(CC5)C4)cn3c2=O)c1)C1CCC1 ; Cn1cnnc1[C@@H](c1cccc(-n2cc3c(C(F)(F)F)cc(CN4CCC5(CC5)C4)cn3c2=O)c1)C1CCC1]

### Synthesis of 163

162a (240 mg) was purified by Prep-Chiral-HPLC separation with the following conditions (Column: CHIRALPAK IC, 2*25 cm, 5 um; Mobile Phase A: Hex (0.5% 2M NH₃-MeOH), Mobile Phase B: EtOH:DCM=1:1; Flow rate: 20 mL/min; Gradient: 50% B to 50% B in 13 min; Wave Length: 220/254 nm; RT1 (min): 7.75, RT2 (min): 10.35, the second peak is product) to afford Compound 163 (104.6 mg, 45.50%) as a yellow solid.

LCMS: (ES, m/z): [M+H]⁺ 537.

H-NMR: (400 MHz, DMSO-d₆, ppm): δ 0.51-0.53 (d, 4H), 1.73-1.80 (m, 7H), 2.05-2.14 (m, 1H), 2.46-2.49 (m, 2H), 2.66-2.68 (m, 2H), 3.19-3.25 (m, 1H), 3.40-3.43 (m, 5H), 4.25-4.27 (d, 1H), 7.04 (s, 1H), 7.18-7.20 (d, 1H), 7.31 (s, 1H), 7.42-7.46 (m, 1H), 7.67-7.73 (m, 3H), 8.33 (s, 1H).

Alternatively, compound 163 may be also prepared in the manner outlined below:

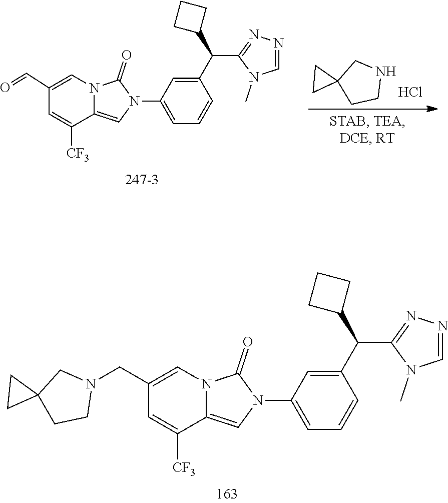

### 1. Synthesis of 163

To a stirred mixture of 247-3 (50 g, 109.784 mmol, 1 equiv) and 5-azaspiro[2.4]heptane (16.00 g, 164.676 mmol, 1.5 equiv) in DCE (500 mL) was added TEA (22.22 g, 219.568 mmol, 2 equiv) at room temperature. The resulting mixture was stirred for 1h at room temperature. To the above mixture was added STAB (46.54 g, 219.568 mmol, 2 equiv) at room temperature. The resulting mixture was stirred for 4h at room temperature. The residue was dissolved in water (1000 mL). The aqueous layer was extracted with CH₂Cl₂ (3×500 mL). The resulting mixture was concentrated under reduced pressure. The residue was purified by reverse flash chromatography with the following conditions: column, C18 silica gel; mobile phase, MeCN in Water (10 mmol/L NH₄HCO₃), 15% to 50% gradient in 45 min; detector, UV 254 nm. This resulted in 163 (25.6175 g, 43.49%) as a yellow solid.

LC-MS-163: (ES, m/z): [M+H]⁺ 537. H-NMR-163: (400 MHz, DMSO-d₆, δ ppm): 0.50-0.52 (m, 4H), 1.68-1.81 (m, 7H) 2.08-2.09 (m, 1H), 2.46-2.51 (m, 2H), 2.67-2.70 (m, 2H) 3.19-3.25 (m, 1H) 3.31-3.43 (m, 5H) 4.25-4.27 (m, 3H), 7.04 (d, 1H), 7.18-7.20 (d, 1H), 7.30 (s, 1H), 7.42-7.46 (m, 1H), 7.67 (m, 1H), 7.70-7.73 (m, 1H), 8.32 (s, 1H).

### Example 164. Synthesis of Compound 164

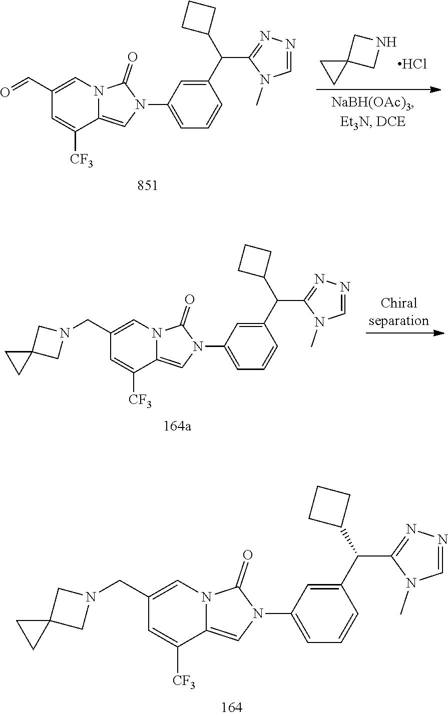

### Synthesis of 164a

To a stirred solution of 85i (800 mg, 1.757 mmol, 1 equiv) and 5-azaspiro [2,3]hexane hydrochloride (421.7 mg, 3,514 mmol, 2 equiv) in DCE (10 mL) were added TEA (355.49 mg, 3.514 mmol, 2 equiv) and NaBH(OAc)3 (744.57 mg, 3.514 mmol, 2 equiv) at room temperature. The resulting mixture was stirred overnight at room temperature. The reaction was quenched by the addition of sat. NH₄Cl (aq.) (15 mL) at room temperature. The resulting mixture was extracted with EtOAc (2×20 mL) and dried over anhydrous Na₂SO₄. After filtration, the filtrate was concentrated under reduced pressure. The crude product (550 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 55% B in 8 min; Wave Length: 220 inn; RT1 (min): 7.65) to afford 164a (400 mg, 42.71%) as a yellow solid.

### Synthesis of 164

The 164a (400 mg) was purified by Chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=I: 1-HPLC; Flow rate: 20 mL/min; Gradient: 40% 3 to 50% B in 16 min; Wave Length: 220/254 min; RT1 (min): 9.69; RT2 (min): 12.92; The first peak was the product; Sample Solvent: EtOH:DCM=1:1-HPLC; Injection Volume: 0.7 mL; Number of Runs: 6) to afford Compound 164 (152.5 mg, 37.71%) as a yellow solid.

LC-MS: (ES, m/z): [M+H]⁺:523

H-NMR: (400 MHz, DMSO-d6, ppm): δ 0.52-0.56 (m, 4H), δ 1.73-1.86 (m, 5H), δ 2.02-2.16 (m, 1H), δ 3.18-3.21 (m, 1H), δ 3.31-3.35 (m, 4H), δ 3.45 (s, 3H), δ 3.51-3.62 (m, 2H), S 4.62-4.64 (d, 1H), δ 7.02 (s, 1H), δ 7.18-7.20 (d, 1H), δ 7.31 (s, 1H), δ 7.42-7.46 (m, 3H), S 7.67-7.73 (m, 3H), δ 8.33 (s, 1H).

### Example 165. Synthesis of Compound 165

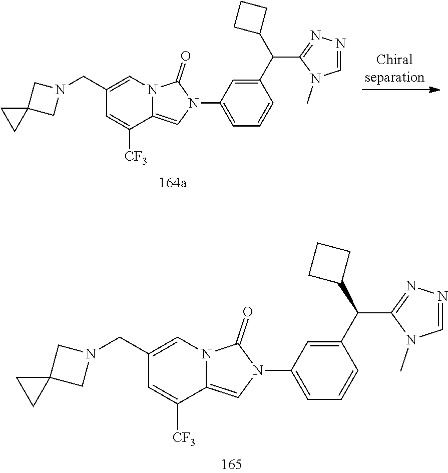 [SMILES: Cn1cnnc1C(c1cccc(-n2cc3c(C(F)(F)F)cc(CN4CC5(CC5)C4)cn3c2=O)c1)C1CCC1 ; Cn1cnnc1[C@@H](c1cccc(-n2cc3c(C(F)(F)F)cc(CN4CC5(CC5)C4)cn3c2=O)c1)C1CCC1]

### Synthesis of 165

The 164a (400 mg, 0,765 mmol, 1 equiv) (400 mg) was purified by Chiral separation with the following conditions (Column: CHIRALPAK TC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 45% B to 50% B in 16 min; Wave Length: 220/254 nm; RT1 (min): 9.69; RT2 (min): 12.92; The second peak was the product; Sample Solvent: EtOH:DCM=1:1-PLC; Injection Volume: 0.7 mL; Number of Runs: 6) to afford Compound 165 (132.5 mg, 33.03%) as a yellow solid.

LC-MS: (ES, m/z): [M+H]⁺:523

H-NMR: (400 MHz, DMSO-d6, ppm): δ 0.52-0.56 (m, 4H), δ 1.73-1.86 (m, 5H), δ 2.02-2.16 (m, 1H), δ 3.18-3.21 (m, 1H), δ 3.31-3.35 (m, 4H), δ 3.45 (s, 3H), δ 3.51-3.62 (m, 2H), S 4.62-4.64 (d, 1H), δ 7.02 (s, 1H), δ 7.18-7.20 (d, 1H), δ 7.31 (s, 1H), δ 7.42-7.46 (m, 3H), S 7.67-7.73 (m, 3H), δ 8.33 (s, 1H).

### Example 166. Synthesis of Compound 166

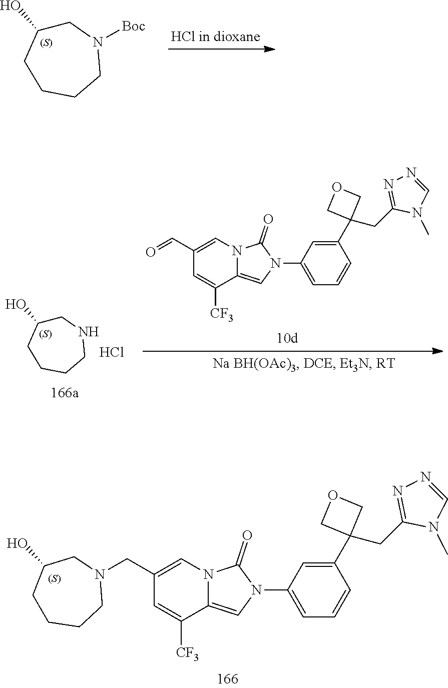

### Synthesis of 166a

Into a 20 mL sealed tube were added tert-butyl (3S)-3-hydroxyazepane-1-carboxylate (500 mg, 2.32 mmol, 1.0 equiv) and HCl (gas) in 1,4-dioxane (5 mL) at room temperature. The resulting mixture was stirred for 2 h at room temperature. The resulting mixture was concentrated under reduced pressure to afford 166a (250 mg, 71%) as an off-white solid.

### Synthesis of 166

To a stirred solution of 10d (150 mg, 0.32 mmol, 1.0 equiv) and 166a (149 mg, 0.98 mmol, 3.0 equiv) in DCE (3 ml) were added Et₃N (99 mg, 0.98 mmol, 3.0 equiv) and NaBH(OAc)₃ (139 mg, 0.66 mmol, 2.0 equiv) at room temperature. The resulting mixture was stirred overnight at room temperature. The reaction was quenched by the addition of sat NH₄Cl (aq.) (10 mL) at room temperature. The aqueous layer was extracted with CH₂Cl₂ (3×10 mL). The resulting mixture was concentrated under reduced pressure. The residue was purified by Prep-TLC (CH₂Cl₂/MeOH=10:1) to afford crude product (100 mg). The crude product (100 mg) was purified by Prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 19% B to 49% B in 8 min; Wave Length: 220 nm; RT1 (min): 7.43) to afford Compound 166 (33 mg, 18%) as a yellow solid.

LC-MS: (ES, m/z): [M+H]⁺ 557

H-NMR: (400 MHz, dmso-d6, δ ppm): 1.35-1.45 (m, 2H), 1.55-1.70 (m, 3H), 1.72-1.82 (m, 1H), 2.36-2.40 (m, 1H), 2.52-2.57 (m, 1H), 2.61-2.78 (m, 2H), 2.96 (s, 3H), 3.43 (s, 2H), 3.53 (s, 2H), 3.60-3.64 (m, 1H), 4.39-4.41 (d, 1H), 4.91-4.96 (m, 4H), 6.88-6.90 (d, 1H), 7.07 (s, 1H), 7.31 (s, 1H), 7.38-7.42 (m, 2H), 7.69 (s, 1H), 7.74-7.75 (d, 1H), 8.20 (s, 1H).

### Example 167. Synthesis of Compound 167

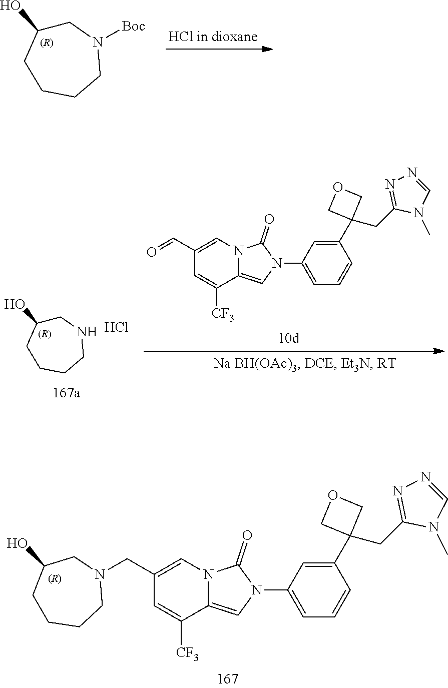

### Synthesis of 167a

Into a 20 mL sealed tube were added tert-butyl (3R)-3-hydroxyazepane-1-carboxylate (500 mg, 232 mmol, 1.0 equiv) and HCl (gas) in 1,4-dioxane (5 mL) at room temperature. The resulting mixture was stirred for 2 h at room temperature under nitrogen atmosphere. The resulting mixture was concentrated under reduced pressure to afford 167a (250 mg, 71%) as an off-white solid.

### Synthesis of 167

To a stirred solution of 10d (150 mg, 0.32 mmol, 1.0 equiv) and 167a (149 mg, 0.98 mmol, 3.0 equiv) in DCE (3 ml) were added Et₃N (99 mg, 0.98 mmol, 3.0 equiv) and NaBH(OAc)3 (139 mg, 0.66 mmol, 2.0 equiv) at room temperature. The resulting mixture was stirred overnight at room temperature. The reaction was quenched by the addition of sat NH₄Cl (aq.) (10 mL) at room temperature. The aqueous layer was extracted with CH₂Cl₂ (3×10 mL). The resulting mixture was concentrated under reduced pressure. The residue was purified by Prep-TLC (CH₂Cl₂/MeOH=10:1) to afford crude product (100 mg). The crude product (100 mg) was purified by Prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 19% B to 49% B in 8 min; Wave Length: 220 nm; RT1 (min): 7.43) to afford Compound 167 (28 mg, 15%) as a yellow solid.

LC-MS: (ES, m/z): [M+H]⁺ 557

H-NMR: (400 MHz, dmso-d6, δ ppm): 1.35-1.45 (m, 2H), 1.55-1.70 (m, 3H), 1.72-1.82 (m, 1H), 2.36-2.40 (m, 1H), 2.52-2.57 (m, 1H), 2.61-2.78 (m, 2H), 2.96 (s, 3H), 3.43 (s, 2H), 3.53 (s, 2H), 3.60-3.64 (m, 1H), 4.39-4.41 (d, 1H), 4.91-4.96 (m, 4H), 6.88-6.90 (d, 1H), 7.07 (s, 1H), 7.31 (s, 1H), 7.38-7.42 (m, 2H), 7.69 (s, 1H), 7.74-7.75 (d, 1H), 8.20 (s, 1H).

### Example 168. Synthesis of Compound 168

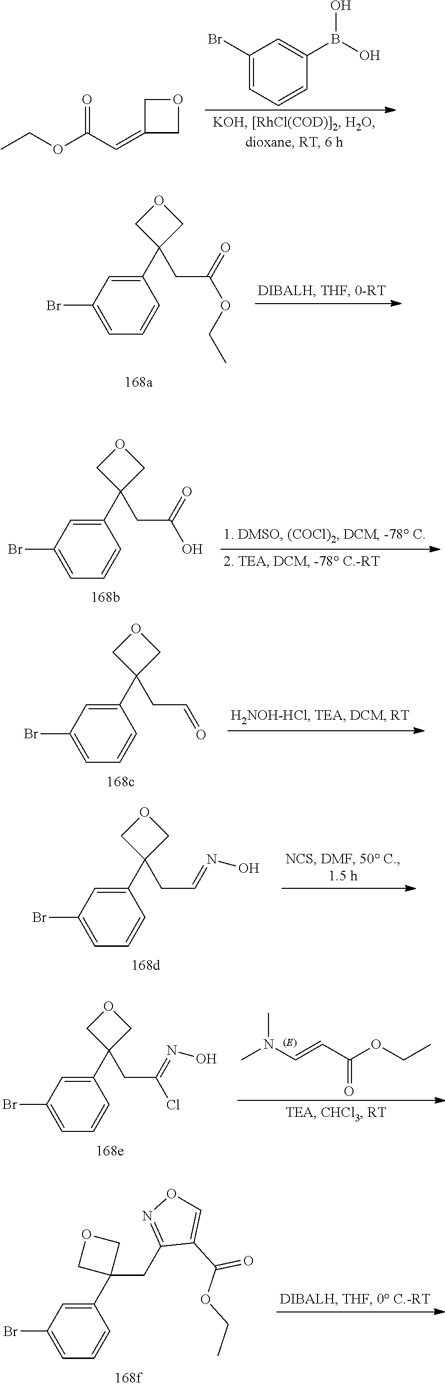

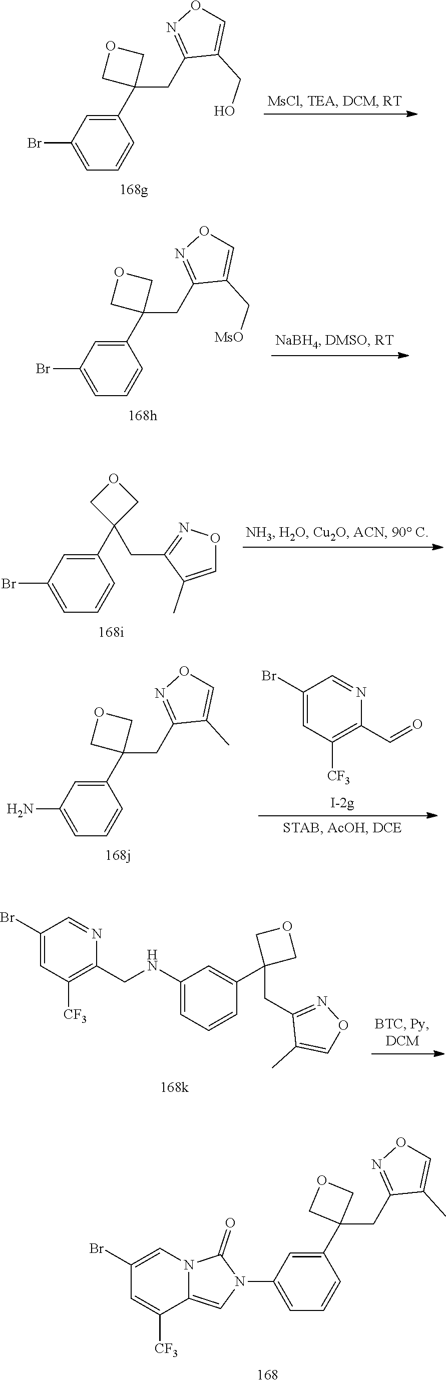

### Synthesis of 168a

Into a 2 L 3-necked round-bottom flask were added bis((1Z,5Z)-cycloocta-1,5-diene); bis(chlororhodium) (2.08 g, 4.221 mmol, 0.02 equiv), dioxane (750 mL), H₂O (150 mL), and KOH (1776 g, 316.558 mmol, 1.5 equiv) at room temperature. The resulting mixture was stirred for 0.5 h at room temperature under nitrogen atmosphere. To the above mixture was added 3-bromophenylboronic acid (50.86 g, 253.247 mmol, 1.2 equiv) and ethyl 2-(oxetan-3-ylidene)acetate (30 g, 211.039 mmol, 1.00 equiv) at room temperature. The resulting mixture was stirred overnight at room temperature. The reaction was quenched by the addition of saturated aqueous NH₄Cl (1000 mL) at room temperature. The aqueous layer was extracted with EtOAc (3×500 mL). The resulting mixture was concentrated under reduced pressure. The residue was purified by silica gel column chromatography, eluted with PE/EA (12:1) to afford 168a (40 g, 63.36%) as a light yellow oil.

### Synthesis of 168b

Into a 500 mL 3-necked round-bottom flask were added 168a (20 g, 66.853 mmol, 1 equiv), THF (200 mL) and DIBALH (33.91 mL, 167.132 mmol, 2.5 equiv) at 0° C. The resulting mixture was stirred overnight at room temperature. The reaction was quenched by the addition of sat. NH₄Cl (aq.) (200 mL) at room temperature. The aqueous layer was extracted with EtOAc (3×150 mL). The resulting mixture was concentrated under reduced pressure. The crude product was purified by reverse phase flash with the following conditions (Column: SNP C18 330 g; Mobile Phase A: Water (0.1% NH₄HCO₃), Mobile Phase B: ACN; Flow rate: 80 mL/min; Gradient: 30% B to 32% B in 15 min; 220 nm; to afford 168b (15 g, 87.26%) as a light brown oil.

### Synthesis of 168c

To a stirred solution of DCM (30 mL) and (COCl)₂ (5.92 g, 46.669 mmol, 1.20 equiv) was added DMSO (0.8 mL) dropwise at −78° C. under nitrogen atmosphere. The resulting mixture was stirred for 20 min at −78° C. under nitrogen atmosphere. To the above mixture was added 168b (10 g, 38.891 mmol, 1 equiv) at −78° C. The resulting mixture was stirred for additional 1 h at −78° C. To the above mixture was added TEA (20.42 g, 201.844 mmol, 5.19 equiv) dropwise at −78° C. The resulting mixture was stirred for additional 10 min at −78° C. The final reaction mixture was stirred overnight at room temperature. The reaction was quenched by the addition of water (100 mL) at room temperature. The aqueous layer was extracted with EtOAc (3×100 mL). The resulting mixture was concentrated under reduced pressure. The crude product was purified by reverse phase flash with the following conditions (Column: SNAP C18 330 g; Mobile Phase A: Water (0.1% NH₄HCO₃), Mobile Phase B: ACN; Flow rate: 80 mL/min; Gradient: 30% B to 32% B in 15 min; 220 nm) to afford 168c (5.5 g 55.43%) as a light brown oil,

### Synthesis of 168d

Into a 250 mL 3-necked round-bottom flask was added 168c (5.5 g, 21.559 mmol, 1 equiv), DCM (60 mL), TEA (6.54 g, 64.677 mmol, 3 equiv) and NH₂OH·HCl (2.95 g, 43.118 mmol, 2.00 equiv) at room temperature. The resulting mixture was stirred overnight at room temperature. The reaction was quenched by the addition of water (100 mL) at room temperature. The aqueous layer was extracted with CH₂Cl₂ (3×50 mL). The resulting mixture was concentrated under reduced pressure. The residue was purified by silica gel column chromatography, eluted with PE/EA (5:1) to afford 168d (5.5 g, 94.44%) as a light yellow oil.

### Synthesis of 168e

Into a 250 mL round-bottom flask were added 168d (5.2 g, 19,250 mmol, 1 equiv), DMF (60 mL), and NCS (3.86 g, 28.875 mmol, 1.5 equiv) at room temperature. The resulting mixture was stirred for 2 h at 50° C. under nitrogen atmosphere. The reaction was quenched by the addition of water (150 mL) at room temperature. The aqueous layer was extracted with EtOAc (3×100 mL). The resulting mixture was concentrated under reduced pressure to afford 168e (6 g, 102.34%) as a light brown oil. The crude product was used in the next step directly without further purification.

### Synthesis of 168f

Into a 250 mL 3-necked round-bottom flask were added 168e (6 g, 19.700 mmol, 1 equiv), CHCl₃ (100 mL), ethyl (2E)-3-(dimethylamino)prop-2-enoate (4.23 g, 29.550 mmol, 1.5 equiv), and TEA (2.99 g, 29.550 mmol, 1.5 equiv) at room temperature. The resulting mixture was stirred overnight at room temperature. The reaction was quenched by the addition of water (50 mL) at room temperature. The aqueous layer was extracted with CH₂Cl₂ (3×50 mL). The resulting mixture was concentrated under reduced pressure. The residue was purified by silica gel column chromatography, eluted with PE/EA (5:1) to afford 168f (2.2 g, 30.49%) as a light brown oil.

### Synthesis of 168g

Into a 100 mL 3-necked round-bottom flask were added 168f (2 g, 5,461 mmol, 1 equiv), THF (20 mL) and DIBAL-H (1.94 g, 13.652 mmol, 2.5 equiv) at 0° C. The resulting mixture was stirred overnight at room temperature. The reaction was quenched by the addition of saturated aqueous NH₄Cl (150 mL) at room temperature. The aqueous layer was extracted with EtOAc (3×100 mL). The resulting mixture was concentrated under reduced pressure. The crude product was purified by reverse phase flash with the following conditions (Column: C18 Column, 330 g; Mobile Phase A: water (10 mmol/L NH₄HCO₃), Mobile Phase B: ACN; Flow rate: 60 mL/min; Gradient: 41% B to 70% B in 8 min, 70% B; Wave Length: 220 nm; RT1 (min): 7.65 to afford 168g (1 g, 52.53%) as a white oil,

### Synthesis of 168h

Into a 100 mL round-bottom flask were added 168g (1 g, 3,085 mmol, 1 equiv), DCM (15 ml), TEA (0.62 g, 6.170 mmol, 2 equiv) and MsCl (0.53 g, 4.627 mmol, 1.5 equiv) at room temperature. The resulting mixture was stirred for 2 h at room temperature. The reaction was quenched by the addition of water (20 mL) at room temperature. The aqueous layer was extracted with CH₂Cl (3×20 mL). The resulting mixture was concentrated under reduced pressure to afford 168b (1 g, 80.59%) as a brown oil.

### Synthesis of 168i

Into a 100 mL round-bottom flask were added 168h (1 g, 2.486 mmol, 1 equiv), DMSO (15 mL), and NaBH₄ (0.19 g, 4.972 mmol, 2 equiv) at room temperature. The resulting mixture was stirred overnight at room temperature under nitrogen atmosphere. The reaction was quenched by the addition of saturated aqueous NH₄Cl (25 mL) at room temperature. The aqueous layer was extracted with EtOAc (3×25 mL). The resulting mixture was concentrated under reduced pressure. The crude product was purified by reverse phase flash with the following conditions (Column: C18 Column, 120 g; Mobile Phase A: Water (10 mmol/L NH₄HCO₃), Mobile Phase B: ACN; Flow rate: 50 mL/min; Gradient: 10% B to 40% B in 10 min; Wave Length: 220 nm; RT1 (min): 10) to afford 168i (400 mg, 52.21%) as a brown solid.

### Synthesis of 168j

Into a 50 mL sealed tube were added 168i (400 mg, 1.298 mmol, 1 equiv), ACN (8 mL), NH₃·H₂O (8 mL) and Cu₂O (371.45 mg, 2.596 mmol, 2 equiv) at room temperature. The resulting mixture was stirred overnight at 90° C. under nitrogen atmosphere. The resulting mixture was filtered; the filter cake was washed with MeCN (3×10 mL). The filtrate was concentrated under reduced pressure. The residue was purified by Prep-TLC eluted with CH₂Cl₂/MeOH (10:1) to afford 168j (110 mg, 34.69%) as a light yellow solid.

### Synthesis of 168k

Into a 8 mL sealed tube were added 168j (20 mg, 0.082 mmol, 1 equiv), DCE (1 mL), I-2g (22.87 mg, 0.090 mmol, 1.1 equiv), AcOH (4.92 mg 0.082 mmol, 1 equiv), and STAB (34.70 mg, 0.164 mmol, 2 equiv) at room temperature. The resulting mixture was stirred overnight at room temperature. The resulting mixture was concentrated under reduced pressure. The residue was purified by Prep-TLC eluted with CH₂Cl₂/MeOH (10:1) to afford 168k (21 mg, 53.18%) as a light yellow solid.

### Synthesis of 168

Into a 8 mL sealed tube were added 168k (21 mg, 0.044 mmol, 1 equiv), DCM (1 mL), Pyridine (17.22 mg, 0.220 mmol, 5 equiv) and Triphosgene (9.04 mg, 0.031 mmol, 0.7 equiv) at room temperature. The resulting mixture was stirred for 0.5 h at room temperature under nitrogen atmosphere. The reaction was quenched by the addition of NaHCO₃ (aq.) (10 mL) at room temperature. The aqueous layer was extracted with EtOAc (3×10 mL). The resulting mixture was concentrated under reduced pressure. The crude product (20 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 41% B to 70% B in 8 min, 70% B; Wave Length: 254 nm; RT1 (min): 7.65) to afford Compound 168 (12.0 mg, 54.22%) as a Brown yellow solid.

LC-MS: (ES, m/z): [M+H]⁺ 508

H-NMR: 1H NMR (300 MHz, CD₃OD-d4, ppm, δ) 1.35 (s, 3H), 3.48 (s, 2H), 4.99-5.01 (d, 2H), 5.05-5.06 (d, 2H), 6.99-7.00 (d, 1H), 7.08 (s, 1H), 7.16 (s, 1H), 7.36 (s, 1H), 7.44-7.48 (m, 1H), 7.62-7.64 (d, 1H), 7.97 (s, 1H), 8.18 (s, 1H).

### Example 169. Synthesis of Compound 169

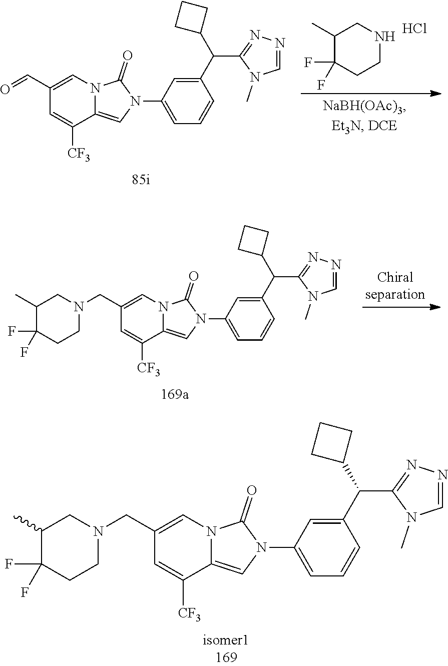

### Synthesis of 169a

To a stirred solution of 85i (1.5 g, 3.296 mmol, 1 equiv), 4,4-difluoro-3-methylpiperidine hydrochloride (680 mg, 3.955 mmol, 1.2 equiv) in DCE (15 mL) were added Et₃N (0.67 g, 6.588 mmol, 2 equiv) and NaBH(OAc)₃ (1.40 g, 6.588 mmol, 2 equiv) at room temperature. The resulting mixture was stirred overnight at room temperature. The reaction was quenched by the addition of sat. NH₄Cl (aq.) (30 mL) at room temperature. The resulting mixture was extracted with EtOAc (3×40 mL) and dried over anhydrous Na₂SO₄. After filtration, the filtrate was concentrated under reduced pressure. The crude product (800 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 62% B in 7 min; Wave Length: 220 nm; RT1 (min): 7.97) to afford 169a (500 mg, 25.89%) as a yellow solid.

### Synthesis of 169

The 169a (500 mg, 0.870 mmol, 1 equiv) was purified by Chiral separation with the following conditions (Column: CHIRALPAK TC, 2*25 cm, 5 μm; Mobile Phase A: Hex (0.5% 2M NH₃-MeOH)-PLC, Mobile Phase B: EtOH:DCM=1:1-HPLC; Flow rate: 20 mL/min; Gradient: 42% B to 50% B in 20 min; Wave Length: 220/254 nm; RT (min): 7.21; Sample Solvent: EtOH:DCM=1:1-HPLC; injection Volume: 0.5 mL; Number of Rums: 8) to afford Compound 169 (72.5 mg, 14.40%) as a yellow solid.

LC-MS: (ES, m/z): [M+H]⁺:575

H-NMR: (400 MHz, DMSO-d6, ppm): δ 1.01-1.03 (d, 3H), δ 1.73-1.74 (d, 1H), δ 1.81-1.98 (m, 5H), δ 2.01-2.21 (m, 4H), δ 2.23-2.24 (d, 1H), δ 2.31-2.34 (m, 1H), δ 2.81-2.95 (m, 2H), δ 3.42-3.42 (d, 2H), δ 3.68 (s, 3H), δ 4.30-4.31 (d, 1H), δ 7.11-7.13 (d, 1H), δ 7.26 (s, 1H), S 7.46-7.47 (d, 1H), δ 7.62-7.75 (m, 3H), δ 8.41 (s, 1H).

### Example 170. Synthesis of Compound 170

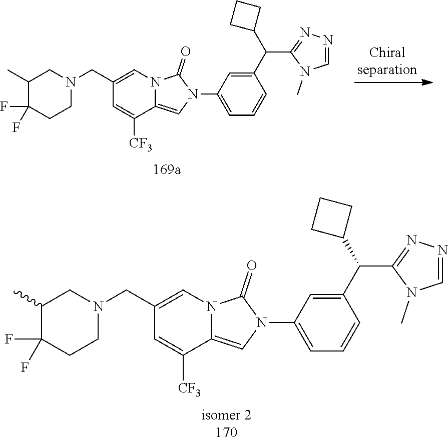 [SMILES: CC1CN(Cc2cc(C(F)(F)F)c3cn(-c4cccc(C(c5nncn5C)C5CCC5)c4)c(=O)n3c2)CCC1(F)F ; CC1CN(Cc2cc(C(F)(F)F)c3cn(-c4cccc([C@@H](c5nncn5C)C5CCC5)c4)c(=O)n3c2)CCC1(F)F]

### Synthesis of 170

The 169a (500 mg, 0.870 mmol, 1 equiv) was purified by Chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 50% B in 20 min; Wave Length: 220/254 nm; RT (min): 8.78; Sample Solvent: EtOH:DCM=1:1-HPLC; Injection Volume: 0.5 mL; Number of Runs: 8) to afford Compound 170 (82.9 mg 16.45%) as a yellow solid,

LC-MS: (ES, m/z): [M+H]⁺:575

H-NMR: (400 MHz, DMSO-d6, ppm): δ 1.01-1.03 (d, 3H), δ 1.73-1.74 (d, 1H), δ 1.81-1.98 (m, 5H), δ 2.01-2.21 (m, 4H), δ 2.23-2.24 (d, 1H), δ 2.31-2.34 (m, 1H), δ 2.81-2.95 (m, 2H), δ 3.42-3.42 (d, 2H), δ 3.68 (s, 3H), δ 4.30-4.31 (d, 1H), δ 7.11-7.13 (d, 1H), δ 7.26 (s, 1H), δ 7.46-7.47 (d, 1H), δ 7.62-7.75 (m, 3H), δ 8.41 (s, 1H).

### Example 171. Synthesis of Compound 171

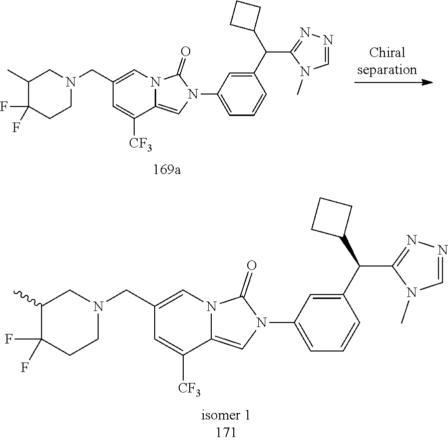 [SMILES: CC1CN(Cc2cc(C(F)(F)F)c3cn(-c4cccc(C(c5nncn5C)C5CCC5)c4)c(=O)n3c2)CCC1(F)F ; CC1CN(Cc2cc(C(F)(F)F)c3cn(-c4cccc([C@H](c5nncn5C)C5CCC5)c4)c(=O)n3c2)CCC1(F)F]

### Synthesis of 171

The 169a (500 mg, 0.870 mmol, 1 equiv) was purified by Chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 45% B to 50% B in 20 min; Wave Length: 220/254 nm; RT (min): 9.88; Sample Solvent: EtOH:DCM=1:1-HPLC; Injection Volume: 0.5 mL; Number of Runs: 8) to afford Compound 171 (83.9 mg, 16.73%) as a yellow solid.

LC-MS: (ES, m/z): [M+H]⁺:575

H-NMR: (400 MHz, DMSO-d6, ppm): δ 1.01-1.03 (d, 3H), δ 1.73-1.74 (d, 1H), δ 1.81-1.98 (m, 5H), δ 2.01-2.21 (m, 4H), δ 2.23-2.24 (d, 1H), δ 2.31-2.34 (m, 1H), δ 2.81-2.95 (m, 2H), δ 3.42-3.42 (d, 2H), δ 3.68 (s, 3H), δ 4.30-4.31 (d, 1H), δ 7.11-7.13 (d, 1H), δ 7.26 (s, 1H), δ 7.46-7.47 (d, 1H), δ 7.62-7.75 (m, 3H), δ 8.41 (s, 1H).

### Example 172. Synthesis of Compound 172

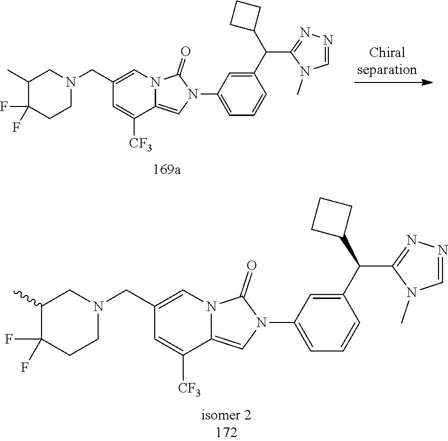 [SMILES: CC1CN(Cc2cc(C(F)(F)F)c3cn(-c4cccc(C(c5nncn5C)C5CCC5)c4)c(=O)n3c2)CCC1(F)F ; CC1CN(Cc2cc(C(F)(F)F)c3cn(-c4cccc([C@H](c5nncn5C)C5CCC5)c4)c(=O)n3c2)CCC1(F)F]

### Synthesis of 172

The 169a (500 mg, 0.870 mmol, 1 equiv) was purified by Chiral separation with the following conditions (Column: CHIRALPAK IC, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 47% B to 50% B in 20 min; Wave Length: 220/254 nm; RT (min): 14.73; Sample Solvent: EtOH:DCM=1:1-HPLC; Injection Volume: 0.5 mL; Number of Runs: 8) to afford Compound 172 (88.6 mg, 17.72%) as a yellow solid.

LC-MS: (ES, m/z): [M+H]⁺:575

H-NMR: (400 MHz, DMSO-d6, ppm): δ 1.01-1.03 (d, 3H), δ 1.73-1.74 (d, 1H), δ 1.81-1.98 (m, 5H), δ 2.01-2.21 (m, 4H), δ 2.23-2.24 (d, 1H), δ 2.31-2.34 (m, 1H), δ 2.81-2.95 (m, 2H), δ 3.42-3.42 (d, 2H), δ 3.68 (s, 3H), δ 4.30-4.31 (d, 1H), δ 7.11-7.13 (d, 1H), δ 7.26 (s, 1H), δ 7.46-7.47 (d, 1H), δ 7.62-7.75 (m, 3H), δ 8.41 (s, 1H).

### Example 173. Synthesis of Compound 173

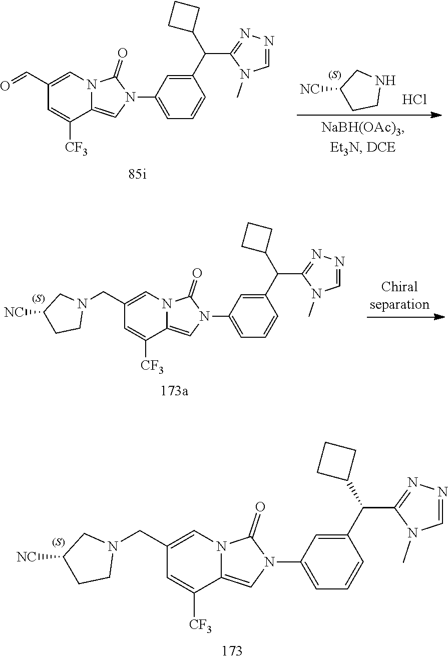

### Synthesis of 173a

To a stirred solution of 85i (800 mg, 1.757 mmol, 1 equiv) and (s)-pyrrolidine-3-carbonitrile hydrochloride (463.73 mg, 3.514 mmol, 2 equiv) in DCE (10 mL) were added Et₃N (355.49 mg 3.514 mmol, 2 equiv) at room temperature under nitrogen atmosphere. The resulting mixture was stirred for 1b at room temperature. To the above mixture was added NaBH(OAc)₃ (744.57 mg 3.514 mmol, 2 equiv) at room temperature. The resulting mixture was stirred for an additional 3h at room temperature. The reaction was quenched with saturated aqueous NH₄Cl at room temperature. The aqueous layer was extracted with DCM (2×20 mL). The organic layers were concentrated under reduced pressure. The residue was purified by Prep-TLC (DCM/MeOH 18:1) to afford the crude product. The crude product (700 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 49% B in 9 min, 49% B; Wave Length: 220 nm; RT1 (min): 8.87) to afford 173a (350 mg, 35.34%) as a yellow solid.

### Synthesis of 173

The 173a (350 mg, 0.654 mmol, 1 equiv) was purified by chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 65% B to 65% B in 14 min; Wave Length: 220/254 nm; RT1 (min): 7.06; RT2 (min): 10.54, the first peak is product) to afford Compound 173 (147.4 mg, 41.90%) as a yellow solid.

LC-MS: (ES, m/z): [M+H]⁺ 536

H-NMR: (400 MHz, DMSO-d₆, δ ppm): 1.70-1.83 (m, 5H), 1.95-2.01 (m, 1H), 2.08-2.13 (m, 1H), 2.13-2.25 (m, 1H), 2.50-2.51 (m, 1H), 2.68-2.82 (m, 3H), 3.24-3.30 (m, 2H), 3.43-3.44 (m, 5H), 4.25-4.27 (d, 2H), 7.03 (s, 1H), 7.18-7.20 (m, 1H), 7.32 (s, 1H), 7.42-7.46 (m, 1H), 7.68-7.74 (m, 3H), 8.33 (s, 1H).

### Example 174. Synthesis of Compound 174

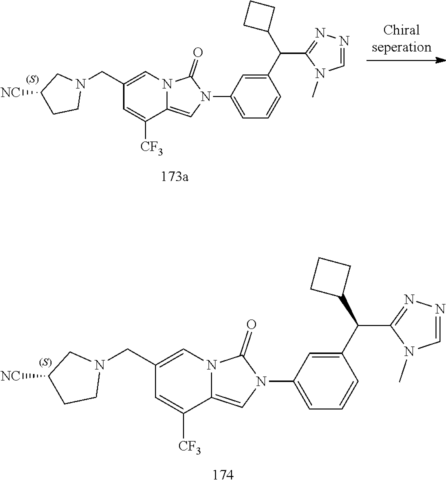

### Synthesis of 174

The 173a (350 mug, 0.654 mmol, 1 equiv) was purified by chiral separation with the following conditions (Column: CHIRALPAK TC, 2*25 cm, 5 μm; Mobile Phase A: Hex (0.5% 2M NH₃-MeOH), Mobile Phase B: EtOH:DCM=1:1; Flow rate: 20 mL/min; Gradient: 65% B to 65% B in 14 min; Wave Length: 220/254 un; RT1 (min): 7.06; RT2 (min): 10.54, the second peak is product) to afford Compound 174 (140.8 mg, 39.87%) as a yellow solid.

LC-MS: (ES, m/z): [M+H]⁺ 536

H-NMR: (400 MHz, DMSO-d₆, δ ppm): 1.70-1.83 (m, 5H), 1.95-2.01 (m, 1H), 2.08-2.13 (m, 1H), 2.13-2.25 (m, 1H), 2.50-2.51 (m, 1H), 2.68-2.82 (m, 3H), 3.24-3.30 (m, 2H), 3.43-3.44 (m, 5H), 4.25-4.27 (d, 2H), 7.03 (s, 1H), 7.18-7.20 (m, 1H), 7.32 (s, 1H), 7.42-7.46 (m, 1H), 7.68-7.74 (m, 3H), 8.33 (s, 1H).

### Example 175. Synthesis of Compound 175

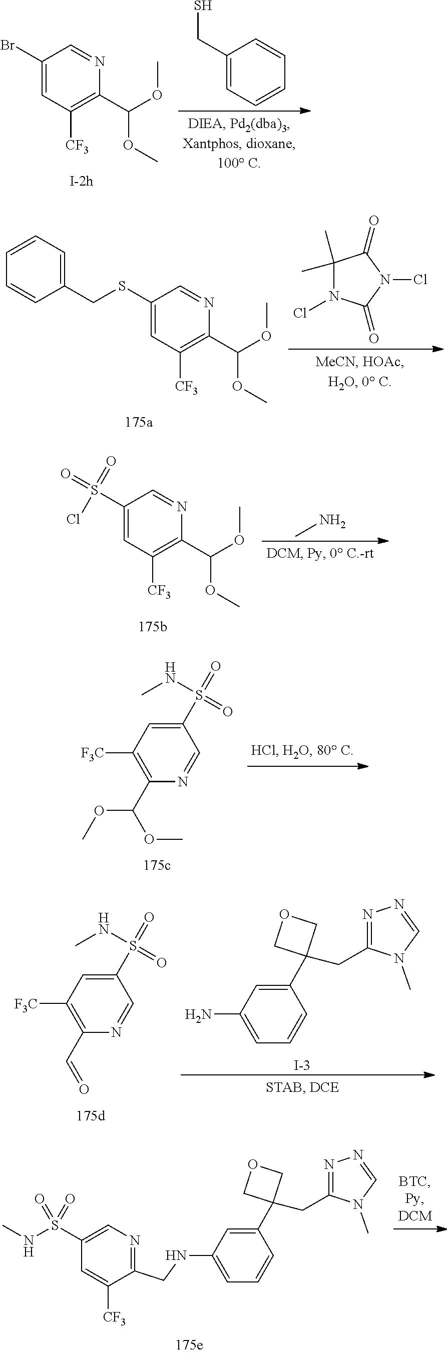

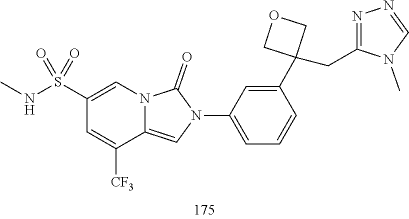 [SMILES: CNS(=O)(=O)c1cc(C(F)(F)F)c2cn(-c3cccc(C4(Cc5nncn5C)COC4)c3)c(=O)n2c1]

### Synthesis of 175a

Into a 40 mL vial were added I-2h (1 g, 3.333 mmol, 1 equiv), benzyl mercaptan (0.62 g, 5.000 mmol, 1.5 equiv), DIEA (0.86 g, 6.666 mmol, 2 equiv), XantPhos (0.39 g, 0.667 mmol, 0.2 equiv), Pd₂(dba)₃ (0.31 g, 0,333 mmol, 0.1 equiv), and dioxane (15 mL) at room temperature. The resulting mixture was stirred overnight at 100° C. under nitrogen atmosphere. The resulting mixture was diluted with water (150 mL). The aqueous layer was extracted with EtOAc (3×60 mL). The resulting mixture was concentrated under vacuum. The residue was purified by Prep-TLC (PE/EA 2:1) to afford 175a (750 mg, 60.30%) as an orange oil.

### Synthesis of 175b

Into a 50 ml round-bottom flask were added 175a (750 mg, 2,184 mmol, 1 equiv), ACN (8 mL), HOAc (0.3 mL) and H2O (0.1 mL) at room temperature. To the above mixture was added 1,3-dichloro-5,5-dimethylimidazolidine-2,4-dione (645.53 mg, 3.276 mmol, 1.5 equiv) at 0° C. The resulting mixture was stirred for 5 h at 0° C. The resulting mixture was diluted with water (50 mL). The aqueous layer was extracted with EtOAc (3×30 mL). The resulting mixture was concentrated under vacuum. The crude product 175b (800 mg) obtained as a colorless oil was used in the next step directly without further purification.

### Synthesis of 175c

Into a 50 mL, round-bottom flask were added 175b (800 mg, 2.503 mmol, equiv), DCM (10 mL) and pyridine (593.84 mg, 7.509 mmol, 3 equiv) at room temperature. To the above mixture was added methylamine (5 mL, 2.5 mol/L in THF, 12.515 mmol, 5 equiv) dropwise at room temperature. The resulting mixture was stirred for 3 h at room temperature. The resulting mixture was concentrated under vacuum. The residue was purified by Prep-TLC (DCM/MeOH 40:1) to afford 175c (500 mg, 60.40%) as a colorless oil,

### Synthesis of 175d

Into a 40 mL vial were added 175c (500 mg, 1.591 mmol, 1 equiv) and HC (10 mL, 1 mol/L in H₂O) at room temperature. The resulting mixture was stirred for 3 h at 80° C. The residue was neutralized to pH 8 with sat. NaHCO₃ (aq.) (30 ml). The aqueous layer was extracted with EtOAc (3×20 mL). The resulting mixture was concentrated under vacuum. The residue was purified by Prep-TLC (PE/EA 2:1) to afford 175d (300 mg, 66.79%) as a colorless oil.

### Synthesis of 175e

Into a 25 mL vial were added 175d (240 mg 0.895 mmol, 1 equiv), I-3 (262.32 mg, 1.074 mmol, 1.2 equiv) and DCE (5 mL) at room temperature. To the above mixture was added STAB (379.30 mg, 1.790 mmol, 2 equiv) at room temperature. The resulting mixture was stirred overnight at room temperature. The reaction was quenched with sat. NH₄Cl (aq.) (50 mL) at room temperature. The aqueous layer was extracted with EtOAc (3×30 ml). The resulting mixture was concentrated under vacuum. The residue was purified by Prep-TLC (DCM/MeOH 20:1) to afford 175e (120 mg, 25.66%) as an off-white solid.

### Synthesis of 175

Into a 50 ml round-bottom flask were added 175e (120 mg, 0,242 mmol, 1 equiv), pyridine (152.94 mg, 1.936 mmol, 8 equiv) and DCM (10 mL) at room temperature. To the above mixture was added triphosgene (50.20 mg, 0.169 mmol, 0.7 equiv) at room temperature. The resulting mixture was stirred for 5 min at room temperature. The reaction was quenched with sat. NaHCO₃ (aq.) (50 mL) at room temperature. The aqueous layer was extracted with EtOAc (3×25 mL). The resulting mixture was concentrated under vacuum. The crude product (120 mg) was purified by Prep-HPLC with the following conditions (Column: X Bridge Prep OBD C18 Column, 30*150 mm, 5 μm; Mobile Phase A: water (10 mmol/L NH₄HCO₃), Mobile Phase B: ACN; Flow rate: 60 m/min; Gradient: 15% B to 40% B in 8 min; Wave Length: 220 nm; RT1 (min): 7.85) to afford Compound 175 (30.9 mg, 23.88%) as an orange solid.

LC-MS: (ES, m/z): [M+H]⁺ 523

H-NMR: 1H NMR (400 MHz, DMSO-d6) δ2.99 (s, 3H), δ 3.33 (s, 3H), δ 3.54 (s, 2H), δ 4.91-4.96 (m, 4H), δ 6.93-6.95 (d, 1H), δ 7.11 (s, 1H), δ 7.41-7.45 (m, 2H), δ 7.56 (s, 1H), δ 7.68-7.75 (m, 2H), δ 8.02 (s, 1H), δ 8.21 (s, 1H).

### Example 176. Synthesis of Compound 176

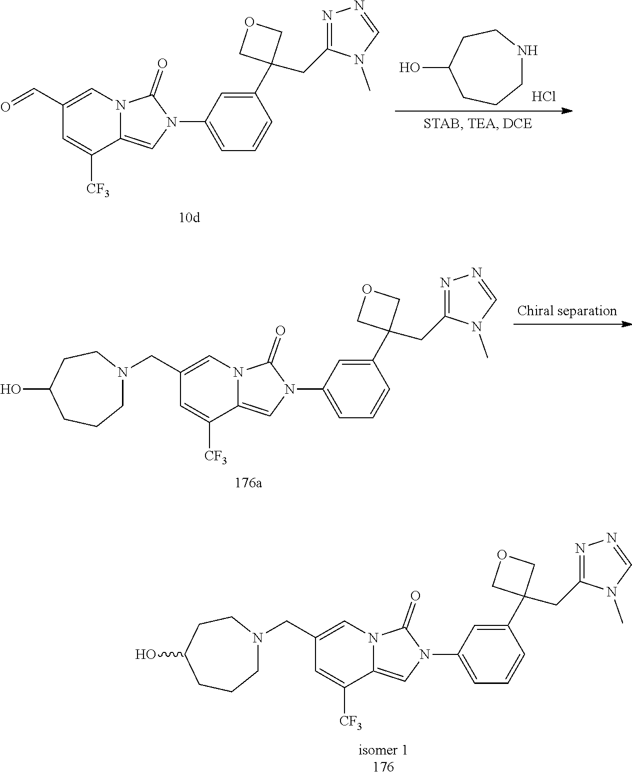 [SMILES: Cn1cnnc1CC1(c2cccc(-n3cc4c(C(F)(F)F)cc(C=O)cn4c3=O)c2)COC1 ; OC1CCCNCC1 ; Cl ; Cn1cnnc1CC1(c2cccc(-n3cc4c(C(F)(F)F)cc(CN5CCCC(O)CC5)cn4c3=O)c2)COC1 ; Cn1cnnc1CC1(c2cccc(-n3cc4c(C(F)(F)F)cc(CN5CCCC(O)CC5)cn4c3=O)c2)COC1]

### Synthesis of 176a

To a stirred mixture of azepan-4-ol hydrochloride (596.69 mg, 3.936 mmol, 3 equiv) and 10d (600 mug, 1.312 mmol, 1.00 equiv) in DCE (6 mL) were added Et₃N (398.20 mg, 3.936 mmol, 3 equiv) and NaBH(OAc)₃ (556.02 mg, 2.624 mmol, 2 equiv) at room temperature. The resulting mixture was stirred overnight at room temperature. The reaction was quenched by the addition of sat NH₄Cl (aq.) (10 mL) at room temperature. The aqueous layer was extracted with CH₂Cl₂ (3×10 mL). The resulting mixture was concentrated under reduced pressure. The residue was purified by Prep-TLC (DCM/MeOH 20:1) to afford 176a (280 mg, 38.35%) as yellow solid,

### Synthesis of 176

176a (200 mg) was purified by Chiral separation with the following conditions (Column: DZ-CHIRALPAK IE-3, 4.6*50 mm, 3.0 μm; Mobile Phase A: MtBE (0.2% DEA): (EtOH:DCM=1:1)=45:55; Flow rate: 1 mL/min; Gradient: 40% B to 50% B; Injection Volume: 5 ul mL in 15 min; 220/254 nm; RT1 14.36 min; RT2: 23.94 min. The first peak was the product). The resulting mixture was concentrated under reduced pressure. This resulted in Compound 176 (74.1 mg, 37.05%) as a yellow solid.

LC-MS: (ES, m/z): [M+H]⁺ 557

H-NMR: (400 MHz, DMSO, δ ppm): 1.71-1.80 (m, 4H), 1.95-1.99 (m, 2H), 2.71-2.76 (m, 4H), 2.96 (s, 3H), 3.49 (s, 2H), 3.68 (s, 2H), 3.91-3.93 (t, 1H), 5.07-5.08 (d, 4H), 6.88-6.89 (m, 1H), 6.90-7.13 (m, 2H), 7.31-7.32 (t, 1H), 7.45-7.49 (m, 1H), 7.62-7.63 (m, 1H), 7.64-7.68 (m, 1H), 8.20 (s, 1H).

### Example 177. Synthesis of Compound 177

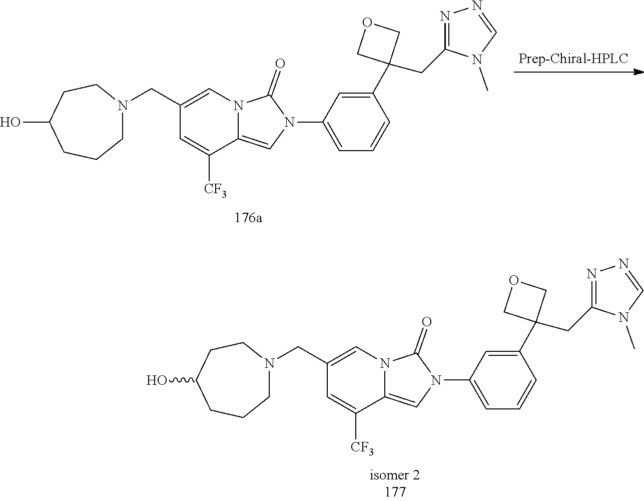 [SMILES: Cn1cnnc1CC1(c2cccc(-n3cc4c(C(F)(F)F)cc(CN5CCCC(O)CC5)cn4c3=O)c2)COC1 ; Cn1cnnc1CC1(c2cccc(-n3cc4c(C(F)(F)F)cc(CN5CCCC(O)CC5)cn4c3=O)c2)COC1]

### Synthesis of 177

176a (200 mg) was purified by Chiral separation with the following conditions (Column: DZ-CHIRALPAK IE-3, 4.6*50 mm, 3.0 μm; Mobile Phase A: MtBE (0.2% DEA): (EtOH:DCM=1:1)=45:55; Flow rate: 1 mL/min; Gradient: 50% B to 50% B; Injection Volume: 5 ul mL in 23 min; 220/254 nm; RT1 14.36 min; RT2: 23.94 min. The second peak was the product). The resulting mixture was concentrated under reduced pressure. This resulted in 177 (76.5 mg, 38.25%) as a yellow solid.

LC-MS: (ES, m/z): [M+H]⁺ 557

H-NMR: (400 MHz, DMSO, δ ppm): 1.71-1.76 (d, 2H), 1.84-1.86 (d, 2H), 1.94-2.04 (m, 2H), 2.78-2.96 (m, 7H), 3.67-3.68 (d, 4H), 3.95-3.97 (t, 1H), 5.07-5.10 (t, 4H), 6.90-6.93 (m, 1H), 7.14 (s, 2H), 7.30-7.31 (m, 1H), 7.45-7.49 (m, 1H), 7.62-7.65 (m, 1H), 7.76 (s, 1H), 8.20 (s, 1H).

### Example 178. Synthesis of Compound 178

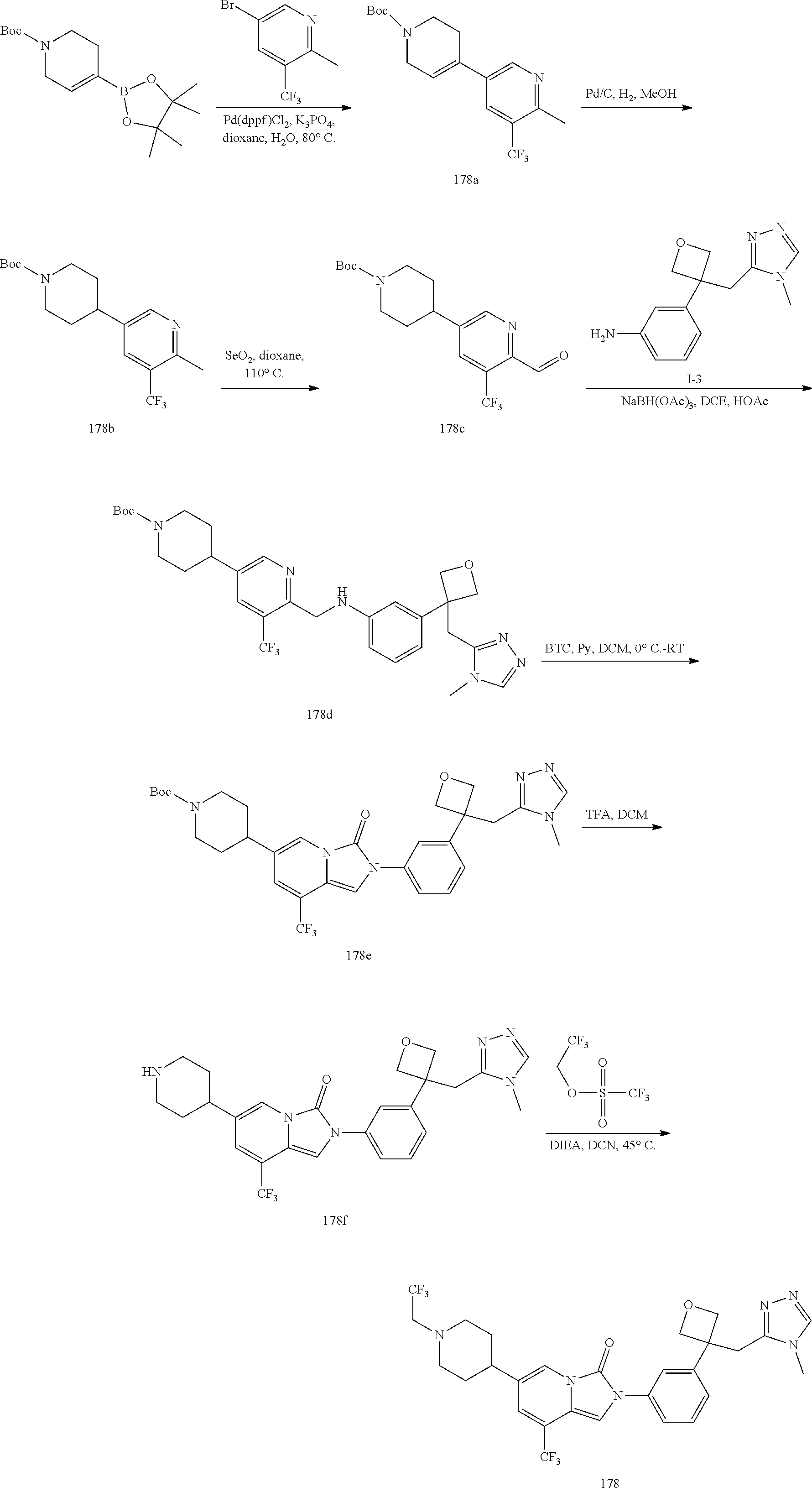

### Synthesis of 178a

Into a 250 mL 3-necked round-bottom flask were added tert-butyl 2-(4,4,5,5-tetramethyl-1,3,2-dioxaborolan-2-yl)-5,6-dihydro-4H-pyridine-1-carboxylate (3.86 g, 12.499 mmol, 1 equiv), 5-bromo-2-methyl-3-(trifluoromethyl)pyridine (3 g, 12.499 mmml, 1 equiv), K₃PO₄ (7.96 g, 37.497 mmol, 3 equiv), Pd(dppf)Cl₂ (1.37 g, 1,875 mmol, 0.15 equiv)₅ dioxane (40 mL), and H₂O (10 mL) at room temperature. The resulting mixture was stirred overnight at 80° C. under nitrogen atmosphere. The reaction was quenched with water (60 ml) at room temperature. The aqueous layer was extracted with EtOEt (3×50 mL). The resulting mixture was concentrated under reduced pressure. The residue was purified by silica gel column chromatography eluted with PE/EA (10:1) to afford 178a (4.1 g, 95.81%) as a yellow oil.

### Synthesis of 178b

Into a 250 mL round-bottom flask were added 178a (4.1 g, 11.976 mmol, 1 equiv), MeOH (60 ml), and Pd/C (10%, 0.4 g) at room temperature. The resulting mixture was stirred overnight at room temperature under hydrogen atmosphere. The resulting mixture was filtered through filter paper then the filter cake was washed with MeOH (3×10 mL). The filtrate as concentrated under reduced pressure. This resulted in 178b (16 g, 80.31%) as an orange oil.

### Synthesis of 178c

Into a 250 mL 3-necked round-bottom flask were added 178b (3.6 g, 10.454 mmol, 1 equiv), SeO₂ (4.64 g, 41.816 mmol, 4 equiv) and dioxane (40 mL) at room temperature. The resulting mixture was stirred overnight at 110° C. The precipitated solids were collected by filtration and washed with dioxane (3×10 mL). The filtrate as concentrated under reduced pressure. The residue was purified by silica gel column chromatography eluted with PE/EA (10:1) to afford 178c (1.2 g, 30.11%) as a brown yellow oil.

### Synthesis of 178d

A solution of 178c (280 mg, 0.781 mmol, 1 equiv) in DCE (5 mL) was treated with 1-3 (190.88 mg, 0.781 mmol, 1 equiv) for 1h at room temperature followed by the addition of HOAc (93.84 mg, 1.562 mmol, 2 equiv) and NaBH(OAc)₃ (331.19 mg, 1.562 mmol, 2 equiv) at room temperature. The resulting mixture was stirred overnight at room temperature. The reaction was quenched with sat. NH₄Cl (aq.) (5 ml) at room temperature. The resulting mixture was extracted with EtOAc (3×5 mL). The combined organic layers were concentrated under reduced pressure. The residue was purified by Prep-TLC (CH₂Cl₂/MeOH 10:1) to afford 178d (240 mg, 52.36%) as a yellow solid.

### Synthesis of 178e

To a stirred solution of 178d (220 mg, 0.375 mmol, 1 equiv) and Pyridine (59.33 mg, 0,750, mmol, 2 equiv) in DCM (4 mL) were added Triphosgene (44.51 mg, 0.150 mmol, 0.4 equiv) at 0° C. under nitrogen atmosphere. The resulting mixture was stirred for 0.5 h at room temperature under nitrogen atmosphere. The reaction was quenched with sat. NaHCO₃ (aq.) (4 ml) at room temperature. The resulting mixture was extracted with EtOAc (3×10 mL). The combined organic layers were concentrated under reduced pressure. The residue was purified by Prep-TLC (CH₂Cl₂/MeOH 10:1) to afford 178e (160 mg, 69.64%) as a yellow solid.

### Synthesis of 178f

Into an 8 mL vial were added 178e (150 mg, 0.245 mmol, 1 equiv), DCM (3 mL) and TFA (I mL) at room temperature. The resulting mixture was stirred for 3 h at room temperature. The resulting mixture was concentrated under vacuum. The residue was purified by Prep-TLC (CH₂Cl₂/MeOH 10:1) to afford 178f (120 mg, 95.63%) as a yellow solid.

### Synthesis of 178

Into an 8 mL vial were added 178f (110 mg, 0.215 mmol, 1 equiv), 2,2,2-trifluoroethyl trifluoromethanesulfonate (99.63 mg, 0.430 mmol, 2 equiv), ACN (2 mL), and DIEA (83.21 mg, 0.645 mmol, 3 equiv) at room temperature. The resulting mixture was stirred overnight at 45° C. under nitrogen atmosphere. The reaction was quenched by the addition of water (10 mL) at room temperature. The aqueous layer was extracted with EtOAc (10:1) (3×10 mL). The resulting mixture was concentrated under reduced pressure. The residue was purified by Prep-TLC (CH₂Cl₂/MeOH 10:1) to afford crude product (100 mg). The crude product (100 mg) was purified by Prep-HPLC with the following conditions (Column: XBridge Prep OBD C18 Column, 30*150 mm, 5 μm; Mobile Phase A: Water (10 mmol/I NH₄HCO₃), Mobile Phase B: MeOH-HPLC; Flow rate: 60 mL/min; Gradient: 50% B to 72% B in 11 min; Wave Length: 220 nm; RT1 (min): 10.85) to afford Compound 178 (47.3 mg, 37.07%) as a yellow solid.

LC-MS: (ES, m/z): [M+H]⁺ 595

H-NMR: (400 MHz, DMSO-d6, ppm): δ 1.59-1.62 (m, 2H), δ 1.74-1.77 (m, 2H), δ 2.42-2.50 (m, 3H), δ 2.97 (s, 3H), δ 3.00-3.03 (m, 2H), δ 3.19-3.25 (m, 2H), δ 3.53 (s, 2H), δ 4.96-4.98 (dd, 4H), δ 6.88-6.90 (d, 1H), δ 7.14 (s, 1H), δ 7.30 (s, 1H), δ 7.38-7.42 (t, 2H), δ 7.50 (s, 1H), δ 7.74-7.76 (dd, 1H), δ 8.20 (s, 1H).

### Example 179. Synthesis of Compound 179

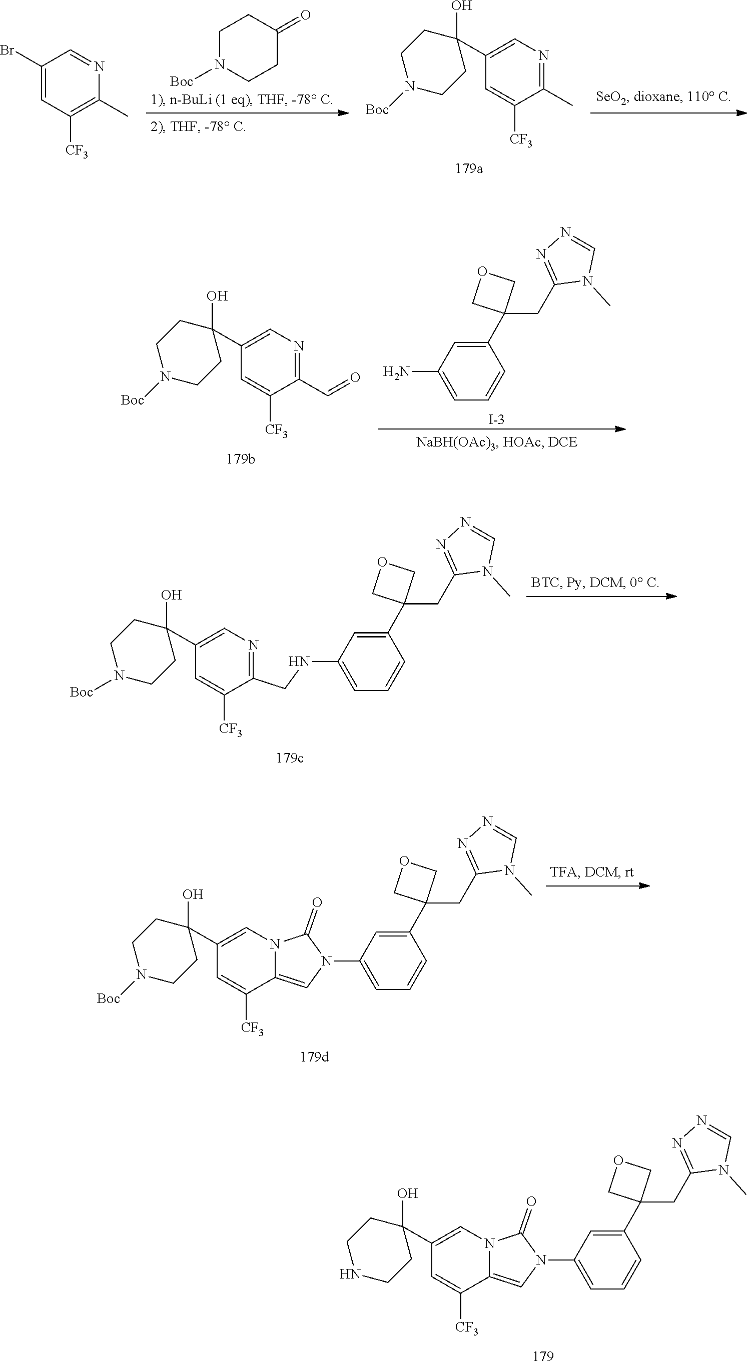

### Synthesis of 179a

To a stirred solution of 5-bromo-2-methyl-3-(trifluoromethyl) pyridine (5 g, 20,831 mmol, 1 equiv) in THE (60 mL) were added n-BuLi (1.60 g, 24.997 mmol, 1.2 equiv) dropwise at −78° C. under nitrogen atmosphere. The resulting mixture was stirred for 1 h at −78° C. under nitrogen atmosphere. To the above mixture was added tert-butyl 4-oxopiperidine-1-carboxylate (4.98 g, 24.997 mmol, 1.2 equiv). The resulting mixture was stirred for additional 11h at 278° C. under nitrogen atmosphere. The reaction was quenched by the addition of sat. NH₄Cl (aq.) (70 mL) at −78° C. The resulting mixture was extracted with EtOAc (2×50 mL) and dried over anhydrous Na₂SO₄. After filtration, the filtrate was concentrated under reduced pressure. The residue was purified by silica gel column chromatography eluted with PE/EA (1:1) to afford 179a (3 g, 36.77%) as a colorless oil,

### Synthesis of 179b

To a stirred solution of 179a (1.5 g, 4.162 mmol, 1 equiv) in dioxane (20 mL) were added SeO₂ (1.85 g, 16.648 mmol, 4 equiv) at room temperature under air atmosphere. The resulting mixture was stirred overnight at 120° C. under air atmosphere. The resulting mixture was filtered through filter paper and the filter cake was washed with 1,4-dioxane (2×20 mL). The filtrate was concentrated under reduced pressure. The reaction was quenched by the addition of Water (40 mL) at room temperature. The resulting mixture was extracted with EtOAc (2×60 mL), dried over anhydrous Na₂SO₄. After filtration, the filtrate was concentrated under reduced pressure. The residue was purified by silica gel column chromatography eluted with PE/EA (5:1) to afford 179b (500 mg, 29.52%) as a colorless oil.

### Synthesis of 179c

To a stirred solution of 179b (500 mg, 1.336 mmol, 1 equiv) and I-3 (391.55 mg, 1.603 mmol, 1.2 equiv) in DCE (10 mL) were added NaBH(OAc)3 (566.14 mg, 2.672 mmol, 2 equiv) and HOAc (80.21 mg, 1.336 mmol, 1 equiv) at room temperature. The resulting mixture was stirred overnight at room temperature. The reaction was quenched by the addition of sat. NH₄Cl (aq.) (20 mL) at room temperature. The resulting mixture was extracted with EtOAc (3×20 mL) and dried over anhydrous Na₂SO₄. After filtration, the filtrate was concentrated under reduced pressure. The residue was purified by Prep-TLC (CH₂Cl₂/MeOH 20:1) to afford 179c (650 mg, 74.29%) as a light yellow oil.

### Synthesis of 179d

To a stirred solution of 179c (600 mg, 0.996 mmol, 1 equiv) in DCM (8 mL) were added pyridine (472.51 mg, 5.976 mmol, 6 equiv) and triphosgene (103.40 mg, 0,349 mmol, 0.35 equiv) at 0° C. under nitrogen atmosphere. The resulting mixture was stirred for 10 min at room temperature under nitrogen atmosphere. The reaction was quenched by the addition of sat NaHCO₃ (aq.) (13 mL) at room temperature. The resulting mixture was extracted with CH₂Cl₂ (2×20 mL) and dried over anhydrous CaCl₂. After filtration, the filtrate was concentrated under reduced pressure. The residue was purified by Prep-TLC (C₁-C₁₂/MeOH 5:1) to afford 179d (450 mg, 66.87%) as a yellow solid.

### Synthesis of 179

To a stirred solution of 179d (400 mg, 0,636 mmol, 1 equiv) in DCM (3 mL) were added TFA (1 mL) at room temperature. The resulting mixture was stirred for 2h at room temperature. The resulting mixture was concentrated under vacuum. The crude product (40 mg) was purified by Prep-HPLC with the following conditions (Column: Xselect CSH F-Phenyl OBD column, 19*250 mm, 5 μm; Mobile Phase A: Water (10 mmol/L NH₄CO₃), Mobile Phase B: ACN; Flow rate: 25 mL/min; Gradient: 40% B to 65% B in 12 min; Wave Length: 220 nm; RT1 (min): 9.87) to afford Compound 179 (250 mg, 74%) as a yellow solid.

LC-MS: (ES, m/z): [M+H]⁺:529

H-NMR: (400 MHz, DMSO-d6, ppm): δ 1.55-1.58 (m, 2H), δ 1.71-1.82 (m, 2H), δ 2.71-2.76 (m, 2H), δ 2.83-2.86 (m, 2H), δ 2.96 (s, 3H), δ 3.52 (s, 2H), δ 4.91-4.93 (m, 2H), δ 4.94-4.96 (d, 2H), δ 5.11 (s, 1H), δ 6.91-6.92 (d, 1H), δ 7.16 (s, 1H), δ 7.28 (s, 1H), δ 7.35-7.39 (m, 2H), δ 7.62-7.63 (d, 1H), δ 7.76-7.78 (d, 1H), δ 8.20 (s, 1H).

### Example 180. Synthesis of Compound 180

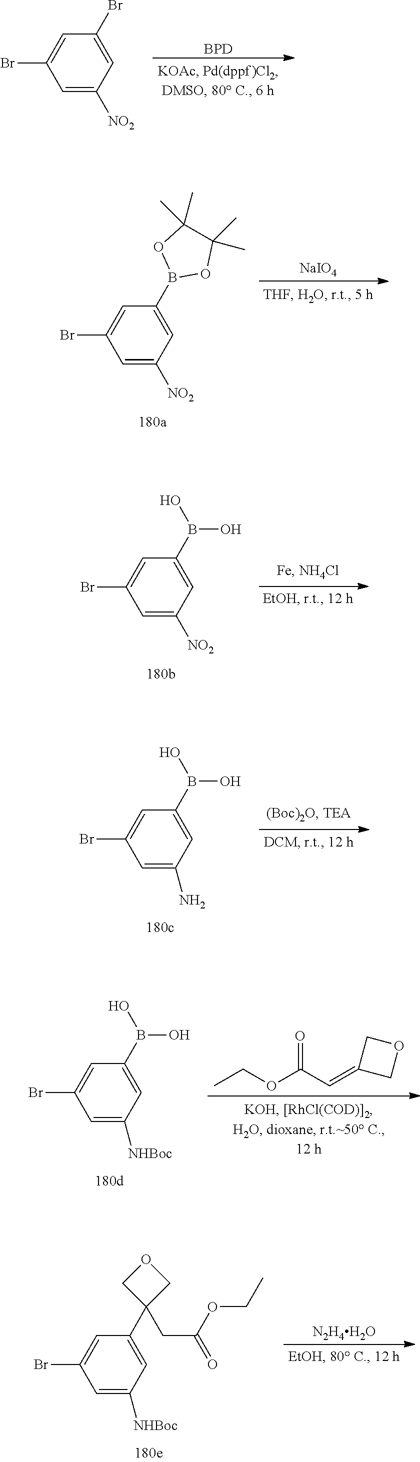

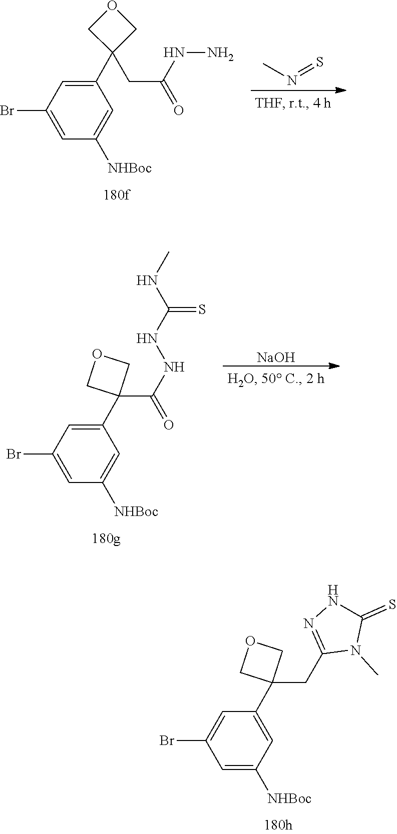

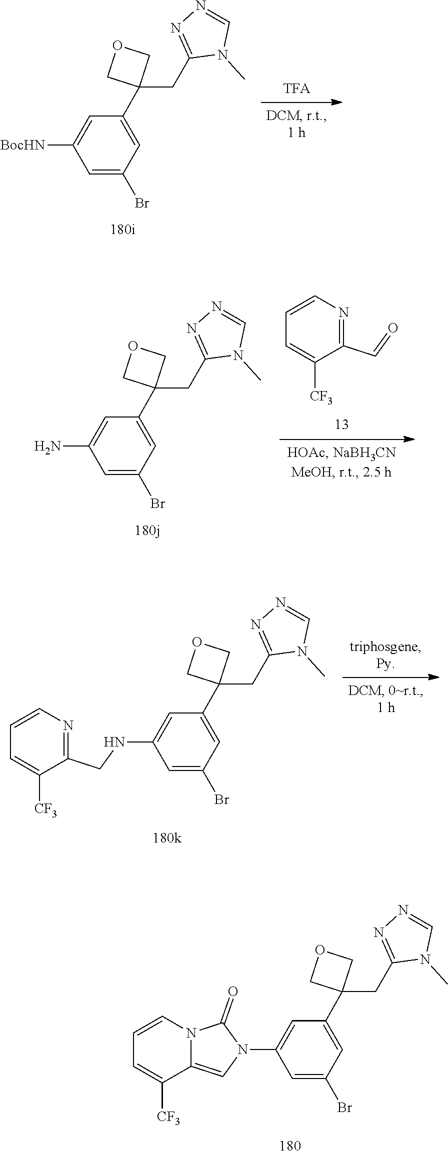

### Synthesis of 180a

To a solution of 1,3-dibromo-5-nitro-benzene (20 g, 71.20 mmol) in dioxane (200 mL) was added bis(pinacolato)diboron (18.08 g, 71.20 mmol), Pd(dppf)Cl₂ (2.60 g, 3.56 mmol) and KOAc (13.98 g, 142.40 mmol). The mixture was stirred at 80° C. for 6 hr under N2 The reaction mixture was diluted with −120 (200 mL) and extracted with DCM (3*200 mL). The combined organic layers were dried over anhydrous sodium sulfate, filtered and the filtrate was concentrated under reduced pressure to give a residue. The residue was purified by column chromatography (SiO₂, petroleum ether/Ethyl acetate=50/1 to 0/1). Compound 180a (13 g, 55.67% yield) was obtained as a white solid.

### Synthesis of 180b

To a solution of 180a (13 g, 39.64 mmol) in THF (90 mL) and H₂O (30 mL) was added NaIO₄ (25.43 g, 118.91 mmol, 6.59 mL) under N₂. The mixture was stirred at r.t. for 5 hr. The reaction mixture was quenched by addition a.q. Na₂SO₃ (200 mL), and then extracted with Ethyl acetate (3*200 mL)) The combined organic layers were dried over anhydrous sodium sulfate, filtered, and the filtrate was concentrated under reduced pressure to give a residue. The residue was purified by column chromatography (SiO₂, Petroleum ether/Ethyl acetate=50/1 to 0/1), Compound 180b (7 g, 71.84% yield) was obtained as a white solid.

### Synthesis of 180c

To a solution of 180b (7 g, 28.48 mmol) in H₂O (20 mL) and EtOH (60 mL) was added Fe (7.95 g, 142.38 mmol) and NH₄Cl (1.52 g, 28.48 mmol) under N₂. The mixture was stirred at r.t. for 12 hr. The reaction mixture was filtered and the filtrate was concentrated under reduced pressure to give a residue. The residue was purified by column chromatography (SiO₂, DCM/MeOH=50/1 to 0/1). Compound 180c (4 g, 65.08% yield) was obtained as a white solid.

### Synthesis of 180d

To a solution of 180c (4 g, 18.53 mmol) in THE (30 mL) and H₂O (15 mL) was added TEA (5.63 g, 55.60 mmol, 7.74 mL) and (Boc)₂₀ (12.13 g, 55.60 mmol, 12.77 mL). The mixture was stirred at r t for 12 hr. The reaction mixture was diluted with H₇20 (20 mL) and extracted with EtOAc (3*20 mL). The combined organic layers were dried over anhydrous sodium sulfate, filtered, and the filtrate was concentrated under reduced pressure to give a residue. The residue was purified by column chromatography (SiO₂, Petroleum ether/Ethyl acetate=50/1 to 0/1), Compound 180d (4 g, 43.04% yield) was obtained as a white solid.

### Synthesis of 180e

To a solution of 180d (3 g, 9.50 mmol) in dioxane (30 mL) was added KOH (1.39 g, 24.69 mmol) in H₂O (6 mL), ethyl 2-(oxetan-3-ylidene)acetate (2.70 g, 18.99 mmol) and chlororhodium;(1Z,5Z)-cycloocta-1,5-diene (936.36 mg, 1.90 mmol). The mixture was stirred at 50° C. for 12 hr under N₂. The reaction mixture was concentrated under reduced pressure to remove solvent to give a residue. The residue was diluted with H₂O (30 mL) and extracted with DCM (3*30 mL). The combined organic layers were dried over anhydrous sodium sulfate, filtered and the filtrate was concentrated under reduced pressure to give a residue. The residue was purified by column chromatography (SiO₂, Petroleum ether/Ethyl acetate-50/1 to 0/1). Compound 180e (2 g, 4.83 mmol, 50.84% yield) was obtained as a white solid.

### Synthesis of 180f

To a solution of 180e (2 g, 4.83 mmol) in EtOH (4 mL) was added NH₂NH₂·H₂O (4.12 g, 69.96 mmol, 4 mL, 85% purity) slowly under N₂. The mixture was stirred at 80° C. for 12 hr under N₂. The reaction mixture was concentrated under reduced pressure to remove solvent at 30° C. Compound 180f (1.8 g, 4.50 mmol, 93.15% yield) was obtained as a white solid.

### Synthesis of 180g

To a solution of 180f (1.8 g, 4.50 mmol) in THF (20 mL) was added methylimino(thioxo)methane (657.61 mg, 8.99 mmol, 614.59 uL) under N₂. The mixture was stirred at r.t. for 4 hr. The reaction mixture was concentrated under reduced pressure to remove solvent. Compound 180g (1.55 g, 72.81% yield) was obtained as a white solid.

### Synthesis of 180h

To a solution of 180g (1.55 g, 3.27 mmol) in H₂O (12 mL) was added NaOH (468.85 mg, 11.72 mmol) under N₂ atmosphere. The mixture was stirred at 50° C. for 2 hr. To the reaction mixture was added 1M HCl to pH<7, and then filtered. The filter cake was dried under reduced pressure to give a residue, Compound 180h (1.3 g, crude) was obtained as a white solid.

### Synthesis of 180i

To a solution of 180h (1.25 g, 2.75 mmol) in EtOH (20 mL) was added Raney-Ni (0.25 g, 2.92 mmol). The mixture was stirred at r.t. for 1 hr under N2. The reaction mixture was filtered through a celite pad and the filtrate was concentrated under reduced pressure to give a residue. The residue was purified by column chromatography (SiO₂, DCM/MeOH=50/1 to 0/1). Compound 180i (0.92 g 79.18% yield) was obtained as a white solid.

### Synthesis of 1801

180i (870 mg, 2.06 mmol) was added to a mixture of DCM (6 mL) and TFA (4.57 g, 40.06 mmol, 2.97 mL). The mixture was stirred at 15° C. for 1 hr under N2. The reaction mixture was concentrated under reduced pressure to remove solvent. The residue was diluted with DCM (50 mL) and added AMBERLYST® A-21 to pH>7. The mixture was filtered and the filtrate was concentrated under reduced pressure to give a residue. Compound 180j (820 mg, crude) was obtained as a white solid,

### Synthesis of 180k

To a solution of 180j (0.65 g, 2.01 mmol) and 3-(trifluoromethyl)pyridine-2-carbaldehyde (387.40 ng, 2.21 mmol) in MeOH (6.5 mL) was added HOAc (12.08 mg, 201.12 umol, 11.50 uL). The mixture was stirred at rt. for 0.5 hr under N₂. Then to the mixture was added NaBH₃CN (379.15 mg, 6.03 mmol). The mixture was stirred at r.t, for 2 hr under N₂. The reaction mixture was quenched by addition of saturated aqueous NH₄Cl (10 mL) under N2, and then extracted with DCM (3*5 mL). The combined organic layers were dried over anhydrous sodium sulfate, filtered, and the filtrate was concentrated under reduced pressure to give a residue. The residue was purified by column chromatography (SiO₂, DCM/MeOH=50/1 to 0/1). Compound 180k (0.5 g, 51.55% yield) was obtained as a white solid.

### Synthesis of 180

To a solution of 180k (0.28 g, 580.56 umol) and Py. (137.77 mg, 1.74 mmol, 140.58 uL) in DCM (3 mL) was added bis(trichloromethyl) carbonate (0.17 g, 572.88 umol) in DCM (0.5 mL) slowly at 0° C. under N2. The mixture was stirred at r.t. for 1 hr under N₂. To the reaction mixture was added saturated aqueous NaHCO₃ (3 mL) slowly at 0° C. under N₂. Then the mixture was stirred at r.t. for 0.5 hr under N₂. The mixture was extracted with EtOAc (3*3 mL). The combined organic layers were dried over anhydrous sodium sulfate, filtered and the filtrate was concentrated under reduced pressure to give a residue. The residue was purified by column chromatography (SiO₂, DCM/MeOH=50/1 to 0/1). The residue (20 mg) was purified by prep-HPLC (column: Waters Xbridge BEH C18 100*30 mm*10 um; mobile phase: [water (NH₄HCO₃)-ACN]; B %: 20%-50%, 8 min). Compound 180 (5.8 mg) was obtained as a yellow solid.

LCMS: (ES, m/z): [M+H]⁺ 508.

H-NMR: (400 MHz, CD₃OD-d₄, ppm): δ 8.25 (s, 1H), 8.06 (t, J=2 Hz, 2H), 7.77 (d, J=7.6 Hz, 1H), 7.43 (s, 1H), 7.20 (s, 1H), 7.10 (d, J=6.8 Hz, 1H), 6.29 (t, J=6.8 Hz, 1H), 4.91 (s, 4H), 3.55 (s, 2H), 3.15 (s, 3H).

### Example 181. Synthesis of Compound 181

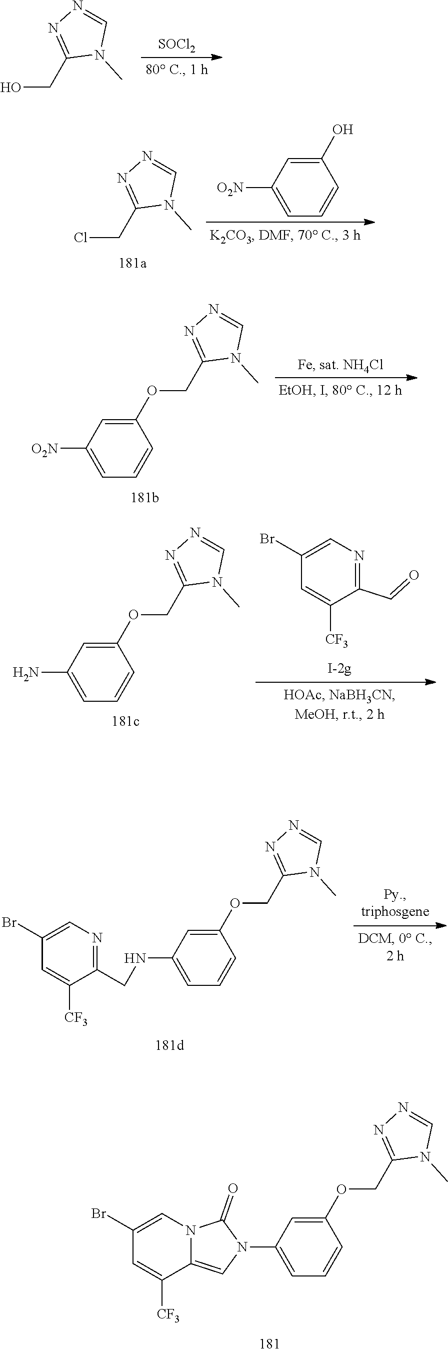

### Synthesis of 181a

A mixture of (4-methyl-1,2,4-triazol-3-yl)methanol (2.90 g, 25.6 mmol) in thionyl chloride (29.0 mL, 399 mmol) was stirred at 80° C. for 1 br under N₂ atmosphere. The mixture was concentrated to afford the residue 181a (4.12 g, 95% yield, as a hydrochloride salt) obtained as a white solid.

### Synthesis of 181b

To a solution of 3-nitrophenol (3.35 mL, 16.86 mmol) and 181a (3.40 g, 20.2 mmol, as a hydrochloride salt) in DMF (20 mL) was added potassium carbonate (11.6 g, 84.3 mmol), the mixture was stirred at 70° C. for 3 hr. Water (50 mL) was added to the reaction mixture and extracted with ethyl acetate (200 mL×2). The combined organic phase was dried with anhydrous sodium sulfate, filtered, and the filtrate was concentrated in vacuum to afford 181b (3.49 g, 88% yield) as a brown solid.

### Synthesis of 181c

A mixture of 181b (3.10 g, 13.2 mmol), ammonium chloride (708 mg, 13.24 mmol), and Fe (3.70 g, 66.1 mmol) in EtOH (30 mL) and water (10 mL) was stirred at 80° C. for 12 hr under N₂ atmosphere. The reaction mixture was filtered, the filtrate was concentrated to give the residue. The residue was purified by normal phase SiO₂ chromatography (0-50% MeOH/EtOAc) to afford 181c (2.0 g, 74% yield) as yellow solid,

### Synthesis of 181d

To a solution of 181c (1.50 g, 7.34 mmol) and I-2g (1.68 g, 6.61 mmol) in MeOH (20 mL) was added acetic acid (7.5 mL g, 131 mmol). The mixture was stirred at 25° C. for 1 hr then sodium cyanoborohydride (1.38 g, 22.0 mmol) was added to the mixture and was stirred at 25° C. for 1 hr. The reaction mixture was dilated with saturated aqueous sodium bicarbonate solution (30 mL) and extracted with EtOAc (30 mL×3). The combined organic layers were dried over anhydrous sodium sulfate, filtered, and concentrated in vacuum. The residue was purified by normal phase SiO2 chromatography (0-10% MeOH/EtOAc) to afford 181d (0.56 g, 17% yield) as orange solid.

### Synthesis of 181

To a solution of 181d (0.56 g, 1.27 mmol) in DCM (20 mL) was added pyridine (408 uL, 5.07 mmol) and bis(trichloromethyl) carbonate (150 mg, 506 umol) at 0° C., the mixture was stirred at 0° C. for 2 hr under N₂. The reaction was quenched with saturated aqueous sodium bicarbonate solution (20 ml). The aqueous layer was extracted with DCM (3×20 mL). The combined organic layer: were dried over anhydrous sodium sulfate, filtered, and the filtrate was concentrated under reduced pressure to give a residue. The residue was purified by normal phase SiO₂ chromatography (0-75% EtOAc/petroleum ether) to afford crude product. The crude product was purified by reverse phase HPLC to afford Compound 181 (35 mg, 5.71% yield) as a yellow solid.

LCMS: (ES, m/z): [M+H]⁺ 468.

H-NMR: (400 MHz, DMSO-d₆, ppm): δ 8.53 (s, 1H), 8.06 (s, 1H), 7.59-7.51 (m, 3H), 7.50-7.43 (m, 1H), 7.18 (s, 1H), 7.13-7.07 (m, 1H), 5.40 (s, 2H), 3.73 (s, 3H)

### Example 182. Synthesis of Compound 182

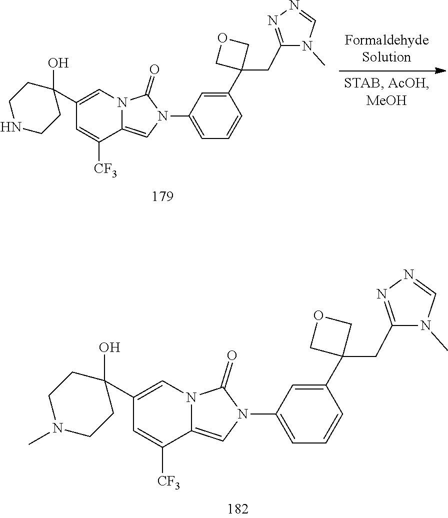

### Synthesis of 182

To a stirred solution of 179 (50 mg, 0.095 mmol, 1 equiv) and Formaldehyde Solution (8.52 mg, 0.285 mmol, 3 equiv) in MeOH (1 mL) were added STAB (40.10 mg, 0,190 mmol, 2 equiv) and HOAc (5.68 mg, 0,095 mmol, 1 equiv) at room temperature. The resulting mixture was stirred for 2h at room temperature. The reaction was quenched by the addition of sat. NaHCO₃ (aq.) (4 mL) at room temperature. The resulting mixture was extracted with CH₂Cl₂ (2×4 mL) and dried over anhydrous CaCl₂. After filtration, the filtrate was concentrated under reduced pressure. The crude product (30 mg) was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 4% B to 15% B in 10 min; Wave Length: 254/220 nm; RT1 (min): 9.5) to afford Compound 182 (5.2 mg, 9.62%) as a yellow solid.

LC-MS: (ES, m/z): [M+H]⁺:544

H-NMR: (400 MHz, DMSO-d6, ppm): δ 1.99-2.05 (m, 2H), δ 2.17-2.23 (m, 2H), δ 2.80-2.81 (d, 3H), δ 2.87-2.90 (m, 3H), δ 3.32-3.33 (d, 2H), δ 3.68 (s, 2H), δ 4.65 (s, 2H), δ 5.03-5.07 (m, 4H), δ 6.96-6.97 (s, 1H), δ 7.15-7.18 (m, 2H), δ 7.29 (s, 1H), δ 7.46-7.52 (m, 1H), δ 7.66-7.67 (d, 1H), δ 7.79 (s, 1H), δ 8.21-8.22 (d, 1H), δ 8.21 (s, 1H).

### Example 183. Synthesis of Compound 183

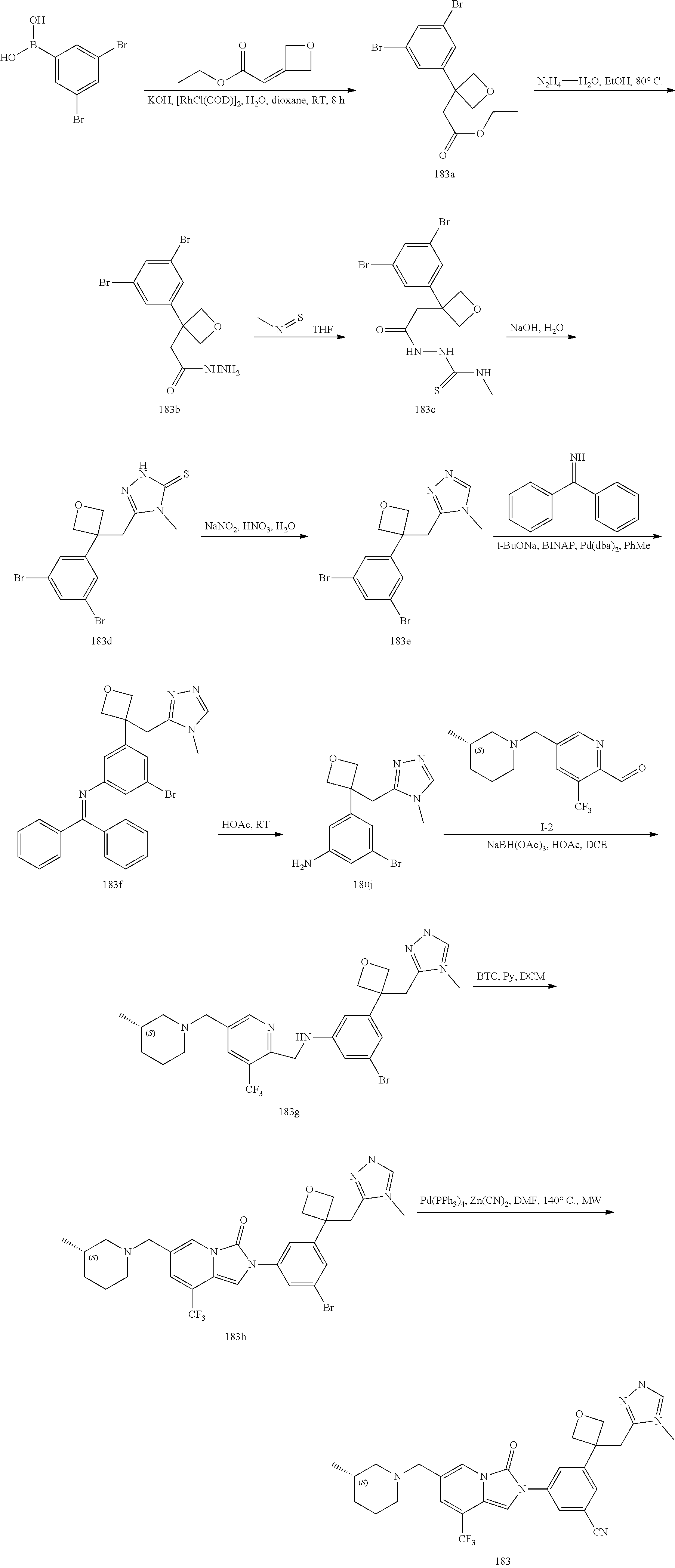

### Synthesis of 183a

Into a 1 L vial were added KOH (6 g, 107.25 mmol, 1.5 equiv) in 1H₂O (47 mL), dioxane (200 mL), and [Rh(COD)Cl]₂ (5.3 g, 10.72 mmol, 0.15 equiv) at room temperature. The mixture was stirred for 30 min at room temperature under nitrogen atmosphere. To the above mixture was added 3,5-dibromophenylboronic acid (20 g, 71.50 mmol, 1.0 equiv) and ethyl 2-(oxetan-3-ylidene) acetate (20.3 g, 143.00 mmol, 2.0 equiv) at room temperature. The resulting mixture was stirred overnight at room temperature. The reaction was quenched by the addition of saturated NH₄Cl (aq.) (1.5 L) at room temperature. The aqueous layer was extracted with EtOAc (3×2 L). The resulting mixture was concentrated under reduced pressure. The residue was purified by silica gel column chromatography, eluted with PE/EtOAc (10:1) to afford 183a (9 g, 33%) as an off-white oil,

### Synthesis of 183b

A solution of 183a (5.9 g, 15.60 mmol, 1.0 equiv) and hydrazine hydrate (6.3 g, 124.84 mmol, 8.0 equiv) in EtOH (30 ml) was stirred overnight at 80° C. The mixture was allowed to cool down to room temperature. The residue was dissolved in water (60 mL). The resulting mixture was filtered by filter paper; the filter cake was washed with water (3×50 mL). The filter cake was concentrated under reduced pressure to afford 183b (5.1 g, 70%) as a white solid.

### Synthesis of 183c

A solution of 183b (5.1 g, 14.01 mmol, 1.0 equiv) and methyl isothiocyanate (2 g, 28.02 mmol, 2.0 equiv) in THF (50 mL) was stirred for 2 h at room temperature. The residue was dissolved in water (100 mL). The resulting mixture was concentrated under reduced pressure. The resulting mixture was filtered through filter paper; the filter cake was washed with water (2×20 mL). The filter cake was dried under reduced pressure to afford 183c (4.8 g, 73%) as a white solid.

### Synthesis of 183d

A solution of 183c (4.8 g, 10.98 mmol, 1.0 equiv) and NaOH (3.5 g, 87.84 mmol, 8.0 equiv) in water (100 mL) was stirred overnight at room temperature. The mixture was acidified to pH 5 with 1M HCl (aq.). The resulting mixture was concentrated under reduced pressure. The resulting mixture was filtered through filter paper; the filter cake was washed with water (3×10 mL). The filter cake was dried under reduced pressure to afford 183d (4.0 g, 85%) as a white solid.

### Synthesis of 183e

A solution of 183d (4 g, 9.54 mmol, 1.0 equiv) in EtOAc (7 mL) was treated with NaNO₂ (6.6 g, 95.43 mmol, 10.0 equiv) followed by the addition of HNO₃ (6 g, 95.43 mmol, 10.0 equiv) dropwise at 0° C. The resulting mixture was stirred for 1h at 0° C. The mixture was neutralized to pH 7 with saturated NaHCO₃ (aq.). The resulting mixture was extracted with EtOAc (3×50 mL). The combined organic layers were washed with water (3×50 mL) and dried over anhydrous Na₂SO₄. The combined organic layers were filtered by filter paper; the filtrate was concentrated under reduced pressure. The residue was purified by silica gel column chromatography, eluted with PE/EA (2:1) to afford 183e (2 g, 54%) as a white solid.

### Synthesis of 183f

To a stirred solution of 183e (2.0 g, 5.17 mmol, LO equiv) and t-BuONa (0.6 g, 6.20 mmol, 1.2 equiv) in toluene (20 mL) were added BINAP (0.24 g, 0.388 mmol, 0.075 equiv), Pd(dba)2 (0.15 g, 0.258 mmol, 0.05 equiv), and diphenylmethanimine (0.94 g, 5.167 mmol, 1 equiv) at room temperature under nitrogen atmosphere. The resulting mixture was stirred overnight at 80° C. under nitrogen atmosphere. The reaction was quenched with water at room temperature. The resulting mixture was extracted with CH₂Cl₂ (3×20 mL). The combined organic layers were washed with water (3×20 mL) and dried over anhydrous Na₂SO₄. The combined organic layers were filtered through filter paper and the filtrate was concentrated under reduced pressure. The residue was purified by silica gel column chromatography, eluted with PE/EA (1:1) to afford 183f (1 g, 40%) as an off-white solid.

### Synthesis of 180j

Into a 250 mL, round-bottom flask were added 183f (880 mg, 1.805 mmol, 1 equiv) and HOAc (10 mL) at room temperature. The resulting mixture was stirred overnight at room temperature under nitrogen atmosphere. The mixture was neutralized to pH 7 with saturated NaHCO₃ (aq.) (250 mL). The aqueous layer was extracted with EtOAc (2×150 mL). The resulting mixture was concentrated under reduced pressure. The residue was purified by Prep-TLC (CH₂Cl₂/MeOH 20:1) to afford 180j (240 mg, 41.13%) as colorless oil,

### Synthesis of 183g

A solution of 180j (240 mg, 0.743 mmol, 1 equiv) in DCE (3 mL) was treated with I-2 (255.12 mg, (0.892 mmol, 1.2 equiv) for 30 min at room temperature under nitrogen atmosphere followed by the addition of NaBH(OAc)3 (314.77 mg, 1.486 mmol, 2 equiv) and HOAc (44.59 mg, 0.743 mmol, 1 equiv) at room temperature. The resulting mixture was stirred overnight at room temperature. The reaction was quenched by the addition of saturated NH₄Cl (aq.) (20 mL) at room temperature. The aqueous layer was extracted with EtOAc (3×7 mL). The resulting mixture was concentrated under reduced pressure. The residue was purified by Prep-TLC (CH₂Cl₂/MeOH 20:1) to afford 183g (300 mg, 68.07%) as a yellow oil.

### Synthesis of 183h

To a stirred solution of 183g (280 mg, 0.472 mmol, 1 equiv) and Pyridine (223.91 mg, 2.832 mmol, 6 equiv) in DCE (5 mL) was added Triphosgene (49.00 mg, 0.165 mmol, 0.35 equiv) at 0° C. The resulting mixture was stirred for 30 min at room temperature under nitrogen atmosphere. The reaction was quenched with saturated NaHCO₃ (aq.) (10 mL) at room temperature. The aqueous layer was extracted with DCM (3×10 mL). The resulting mixture was concentrated under reduced pressure. The residue was purified by Prep-TLC (CH₂Cl₂/MeOH 10:1) to afford 183h (200 mg, 68.43%) as a yellow solid.

### Synthesis of 183

To a stirred solution of 183h (160 mg, 0.258 mmol, 1 equiv) and Pd(PPh₃)₄ (59.69 mg, 0.052 mmol, 0.2 equiv) in DMF (2 ml) was added Zn(CN)₂ (60.67 mg, 0,516 mmol, 2 equiv) at room temperature under nitrogen atmosphere. The final reaction mixture was irradiated with microwave radiation for 2h at 140 degrees C. The reaction was quenched by the addition of saturated NH₄Cl (aq.) (10 mL) at room temperature. The aqueous layer was extracted with EtOAc (4×20 mL). The organic layers were concentrated under reduced pressure. The residue was purified by Prep-TLC (CH₂Cl₂/MeOH 7:1) to afford 183 (150 mg, crude). The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 64% B in 8 min; Wave Length: 220 nm; RT1 (min): 7.27) to afford Compound 183 (82.1 mg, 56.20%) as a yellow solid,

LC-MS: (ES, m/z): [M+H]⁺ 566. H-NMR: (400 MHz, DMSO, δ ppm): 0.82-0.87 (t, 4H), 1.46 (s, 1H), 1.58-1.66 (m, 4H), 1.89 (s, 1H), 2.72-2.77 (t, 2H), 3.22 (s, 3H), 3.25 (s, 2H), 3.58 (s, 2H), 4.91-4.92 (d, 2H), 4.95-4.97 (d, 2H), 7.03 (s, 1H), 7.51 (s, 1H), 7.58-7.59 (t, 1H), 7.66 (s, 1H), 7.97-7.98 (d, 1H), 8.25-8.27 (m, 2H).

### Example 184. Synthesis of Compound 184

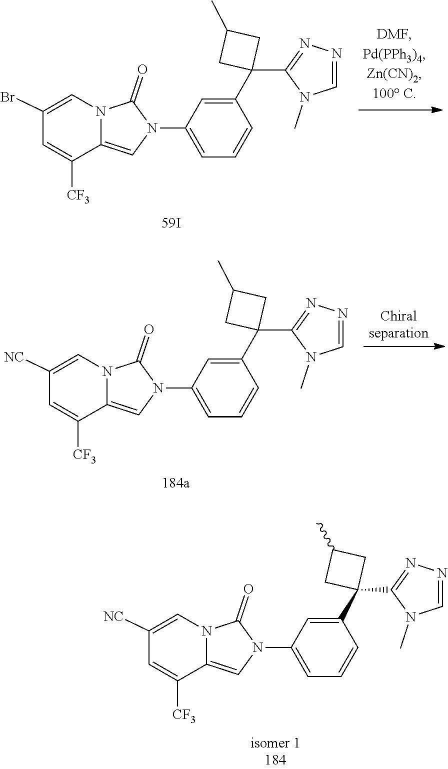

### Synthesis of 184a

To a stirred solution of 59i (500 mg, 0,988 mmol, 1 equiv) and Zn(CN)₂ (231.95 mg, 1,976 mmol, 2 equiv) in DMF (5 mL) was added Pd(PPh₃)4 (114.11 mg, 0.099 mmol, 0.1 equiv) at room temperature under nitrogen atmosphere. The resulting mixture was stirred for 3h at 100° C. under nitrogen atmosphere. The resulting mixture was extracted with EtOAc (2×100 mL). The residue was purified by reverse flash chromatography with the following conditions: column, C18 silica gel; mobile phase, A: water (10 mmol/L NH₄HCO₃), B: MeCN, 10% B to 50% B gradient in 20 min; detector, UV 254 nm. This resulted in 184a (240 mg, 53.63%) as a yellow solid.

### Synthesis of 184

The 184a (240 mg) was purified by chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 90% B to 90% B in 17 min; Wave Length: 220/254 min; RT2 (min): 11.69; RT2 (min): 14.54, the second peak is product) to afford Compound 184 (129.8 mg, 33.30%) as a yellow solid.

LC-MS: (ES, m/z): [M+H]⁺ 453. H-NMR: (400 MHz, DMSO-d6, ppm, δ): 1.08-1.09 (m, 3H), 2.55-2.58 (m, 3H), 2.87-2.89 (m, 2H), 3.23-3.25 (s, 3H), 7.27-7.31 (m, 2H), 7.50-7.52 (m, 1H), 7.54-7.58 (m, 1H), 7.63-7.65 (m, 1H), 7.80 (s, 1H), 8.41 (s, 1H) 8.70 (s, 1H).

### Example 185. Synthesis of Compound 185

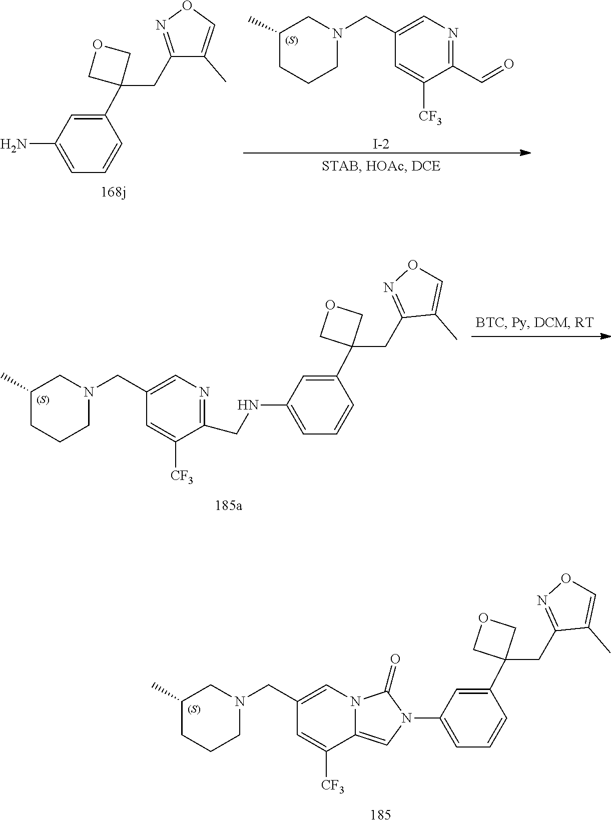

### Synthesis of 185a

To a stirred solution of 168j and 1-2 (105.47 mg, 0.368 mmol, 1 equiv) in DCE (5 mL) was added AcOH (22.12 mg, 0.368 mmol, 1 equiv) and STAB (156.16 mg, 0.737 mmol, 2.00 equiv) at room temperature. The resulting mixture was stirred for 3h at room temperature. The reaction was quenched by the addition of saturated NH₄Cl (aq.) (20 mL) at room temperature. The aqueous layer was extracted with DCM (2×20 mL). The organic layers were concentrated under reduced pressure. The residue was purified by Prep-TLC (DCM/MeOH 20:1) to afford 185a (120 mg, 60.13%) as a light yellow oil.

### Synthesis of 185

A solution of 185a (110 mg, 0.214 mmol, 1 equiv) and Pyridine (169.08 mg, 2.140 mmol, 10 equiv) in DCM (10 mL) was added Triphosgene (31.72 mg, 0.107 mmol, 0.5 equiv) at room temperature under nitrogen atmosphere. The resulting mixture was stirred for 10 min at room temperature. The reaction was quenched with saturated NaHCO₃ (aq.) at room temperature. The aqueous layer was extracted with DCM (2×10 mL). The resulting mixture was concentrated under vacuum. The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 51% B to 81% B in 8 min, 81% B; Wave Length: 220 nm; RT1 (min): 7.50) to afford Compound 185 (49.3 mg, 42.36%) as a yellow solid.

LC-MS: (ES, m/z): [M+H]⁺ 540. H-NMR: (400 MHz, DMSO-d₆, δ ppm): 0.82-0.92 (m, 4H), 1.32 (s, 3H), 1.39-1.47 (m, 1H), 1.47-1.66 (m, 4H), 1.89-1.95 (m, 1H), 2.75-2.77 (m, 2H), 3.30 (s, 2H), 3.41 (s, 2H), 4.84-4.90 (m, 4H), 6.94-6.96 (d, 1H), 7.01 (s, 1H), 7.31 (s, 1H), 7.39-7.43 (m, 1H), 7.46 (s, 1H), 7.75 (s, 1H), 7.76-7.77 (d, 1H), 8.42 (s, 1H).

### Example 186. Synthesis of Compound 186

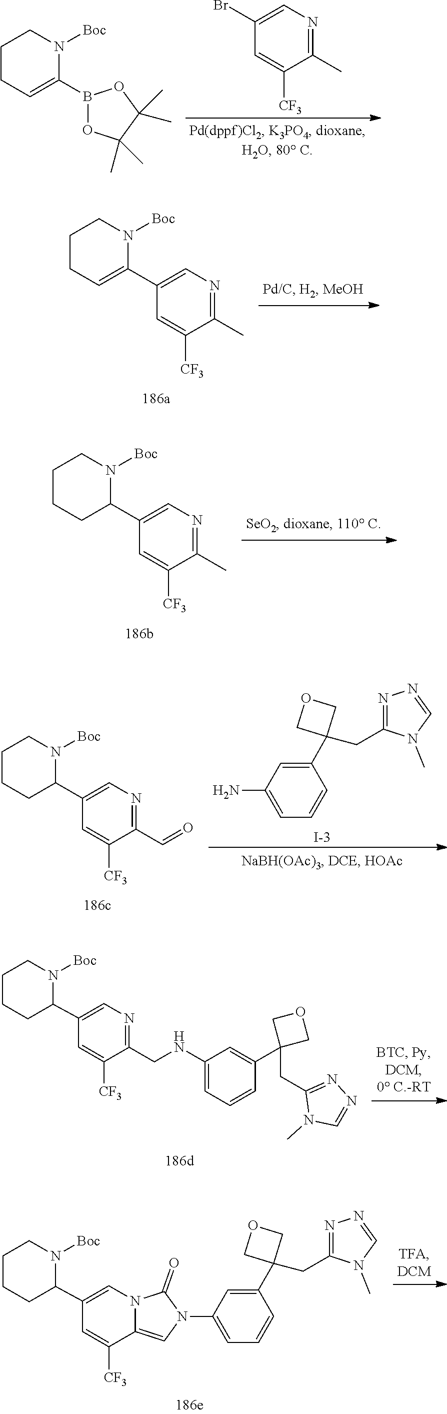

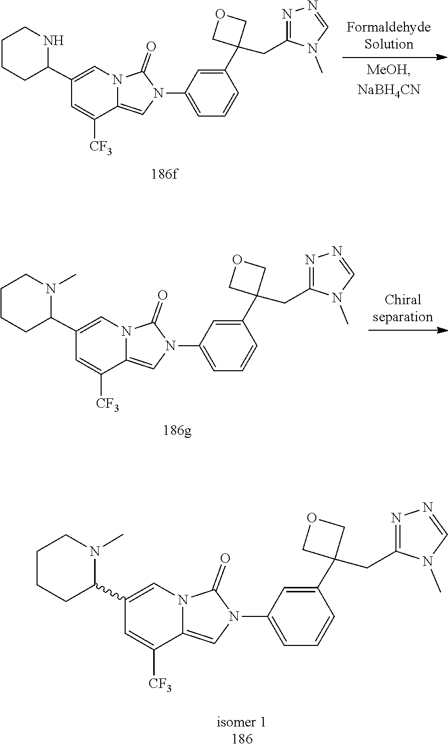 [SMILES: Cn1cnnc1CC1(c2cccc(-n3cc4c(C(F)(F)F)cc(C5CCCCN5)cn4c3=O)c2)COC1 ; CN1CCCCC1c1cc(C(F)(F)F)c2cn(-c3cccc(C4(Cc5nncn5C)COC4)c3)c(=O)n2c1 ; CN1CCCCC1c1cc(C(F)(F)F)c2cn(-c3cccc(C4(Cc5nncn5C)COC4)c3)c(=O)n2c1]

### Synthesis of 186a

Into a 100 mL 3-necked round-bottom flask were added tert-butyl 2-(4,4,5,5-tetramethyl-1,3,2-dioxaborolan-2-yl)-5,6-dihydro-4H-pyridine-1-carboxylate (2 g, 6.468 mmol, 1 equiv), dioxane (20 mL), H₂O (5 mL), 5-bromo-2-methyl-3-(trifluoromethyl)pyridine (2.33 g, 9.702 mmol, 1.5 equiv), K₃PO₄ (4.12 g, 19.404 mmol, 3 equiv), and Pd(dppf)Cl₂ (0.71 g, 0.970 mmol, 0.15 equiv) at room temperature. The resulting mixture was stirred overnight at 80° C. under nitrogen atmosphere. The reaction was quenched by the addition of water (20 mL) at room temperature. The aqueous layer was extracted with EtOAc (3×20 mL). The resulting mixture was concentrated under reduced pressure. The residue was purified by silica gel column chromatography eluted with PE/EA (10:1) to afford 186a (1.8 g, 81.29%) as a light brown solid.

### Synthesis of 186b

To a solution of 186a (1.8 g, 5.258 mmol, 1 equiv) in 20 mL MeOH was added Pd/C (0.56 g) under nitrogen atmosphere in a 100 ml, round-bottom flask. The mixture was hydrogenated at room temperature overnight under hydrogen atmosphere using a hydrogen balloon, filtered through a Celite pad and concentrated under reduced pressure to afford 186b (1.4 g, 77.32%) as a light brown solid.

### Synthesis of 186c

Into a 100 mL round-bottom flask were added 186b (1.4 g, 4.065 mmol, 1 equiv), dioxane (20 mL, 236.082 mmol, 58.07 equiv), and SeO₂ (1.80 g, 16.260 mmol, 4 equiv) at room temperature. The resulting mixture was stirred overnight at 110° C. under nitrogen atmosphere. The resulting mixture was filtered and the filter cake was washed with 1,4-dioxane (3×7 mL). The filtrate was concentrated under reduced pressure. The residue was purified by silica gel column chromatography eluted with PE/EA (6:1) to afford 186c (620 mg, 42.56%) as a light brown solid.

### Synthesis of 186d

Into a 100 mL round-bottom flask were added 186c (600 mg, 1.674 mmol, 1 equiv), DCE (10 mL) and 1-3 (409.03 mg, 1.674 mmol, 1 equiv) at room temperature. The resulting mixture was stirred for 1 h at room temperature. To the above mixture was added HOAc (100.54 mg, 1.674 mmol, 1 equiv) and NaBH(OAc)3 (709.70 mg, 3.348 mmol, 2 equiv) at room temperature. The resulting mixture was stirred overnight at room temperature. The reaction was quenched by the addition of sat. NH₄Cl (aq.) (10 ml) at room temperature. The aqueous layer was extracted with CH₂Cl₂ (3×10 ml). The resulting mixture was concentrated under reduced pressure. The residue was purified by silica gel column chromatography eluted with PE/EA (1:1) to afford 186d (800 mg, 81.45%) as a light brown solid.

### Synthesis of 186e

Into a 100 ml, round-bottom flask were added 186d (780 mg, 1.330 mmol, 1 equiv), DCM (10 mL), Pyridine (525.84 mg, 6.650 mmol, 5 equiv) and Triphosgene (276.18 mg, 0,931 mmol, 0.7 equiv) at 0° C. The resulting mixture was stirred for 0.5 h at room temperature. The reaction was quenched by the addition of sat. Na₃HCO₃ (aq.) (5 mL) at room temperature. The aqueous layer was extracted with CH₂Cl₂ (3×6 mL). The resulting mixture was concentrated under reduced pressure. The residue was purified by Prep-TLC eluted with CH₂Cl₂/MeOH (10:1) to afford 186e (450 mg, 55.24%) as a yellow solid.

### Synthesis of 186f

Into a 100 mL sealed tube were added 1.86e (430 mg, 0.702 mmol, 1 equiv), DCM (6 mL) and TFA (2 mL) at room temperature. The resulting mixture was stirred for 2 h at room temperature. The resulting mixture was concentrated under reduced pressure to afford 186f (200 mg, 55.60%) as a yellow solid,

### Synthesis of 186g

Into a 100 mL round-bottom flask were added 186f (190 mg, 0.371 mmol, 1 equiv), MeOH (5 mL), formaldehyde Solution (16.70 mg, 0.556 mmol, 1.5 equiv) and NaBH₃CN (46.59 mg, 0.742 mmol, 2 equiv) at room temperature. The resulting mixture was stirred overnight at room temperature. The reaction was quenched by the addition of water (10 mL) at room temperature. The aqueous layer was extracted with EtOAc (3×10 mL). The resulting mixture was concentrated under reduced pressure. The residue was purified by Prep-TLC eluted with CH₂Cl₂/MeOH (10:1) to afford 186g (150 mg, 76.84%) as a yellow solid.

### Synthesis of 186

The 186g (150 mg) was purified by Chiral separation with the following conditions (Column: CHIRALPAK IE, 2*25 cm, 5 μm; Mobile Phase A: Hex (0.5% 2M NH₃MeOH)-HPLC, Mobile Phase B: MeOH:DCM=1:1-HPLC; Flow rate: 20 mL/min; Gradient: 30% B to 35% B in 22 min; Wave Length: 220/254 nm; RT1 (min): 18.11; RT2 (min): 20.28; The first peak was the product; Sample Solvent: MeOH:DCM=1:1-HPLC; Injection Volume: 0.3 ml; Number of Runs: 15) to afford Compound 186 (31.0 mg, 20.07%) as a yellow solid.

LC-MS: (ES, m/z): [M+H]⁺ 527

H-NMR: 1H NMR (300 MHz, CD₃OD-d4) δ 1.15-1.38 (m, 1H), 1.39-1.92 (m, 5H), 2.02 (s, 4H), 2.79 (s, 1H), 2.93-2.97 (m, 4H), 3.53 (s, 2H), 4.91-4.96 (m, 4H), 6.89-6.91 (d, 1H), 7.02 (s, 1H), 7.33 (s, 1H), 7.38-7.42 (m, 2H), 7.70 (s, 1H), 7.74-7.76 (m, 1H), 8.20 (s, 1H).

### Example 187. Synthesis of Compound 187

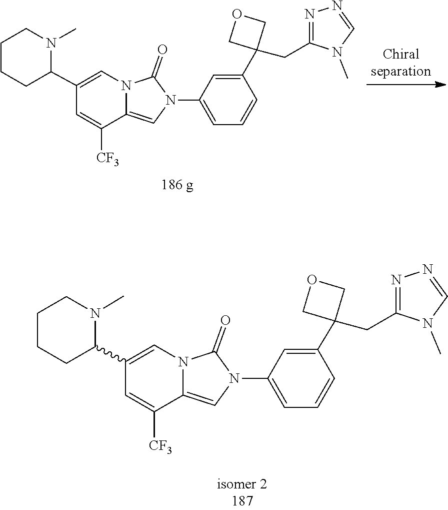 [SMILES: CN1CCCCC1c1cc(C(F)(F)F)c2cn(-c3cccc(C4(Cc5nncn5C)COC4)c3)c(=O)n2c1 ; CN1CCCCC1c1cc(C(F)(F)F)c2cn(-c3cccc(C4(Cc5nncn5C)COC4)c3)c(=O)n2c1]

### Synthesis of 187

The 186g (150 mg) was purified by Chiral separation with the following conditions (Column: CHIRALPAK IE, 2*25 cm, 5 μm; Mobile Phase A: Hex (0.5% 2M NH3-MeOH)-HPLC, Mobile Phase B: MeOH:DCM=1:1-HPLC; Flow rate: 20 mL/min; Gradient: 32% B to 35% 13 in 22 min; Wave Length: 220/254 nm; RT1 (min): 18.11; RT2 (min): 20.28; The second peak was the product Sample Solvent: MeOH:DCM=1:1-HPLC; Injection Volume: 0.3 mL; Number of Runs: 15) to afford Compound 187 (40.1 mg, 26.33%) as a yellow solid.

LC-MS: (ES, m/z): [M+H]⁺ 527

H-NMR: 1H NMR (300 MHz, CD₃OD-d4) S 1.15-1.38 (m, 1H), 1.39-1.92 (m, 5H), 2.02 (s, 4H), 2.79 (s, 1H), 2.93-2.97 (m, 4H), 3.53 (s, 2H), 4.91-4.96 (m, 4H), 6.89-6.91 (d, 1H), 7.02 (s, 1H), 7.33 (s, 1H), 7.38-7.42 (m, 2H), 7.70 (s, 1H), 7.74-7.76 (m, 1H), 8.20 (s, 1H).

### Example 188. Synthesis of Compound 188

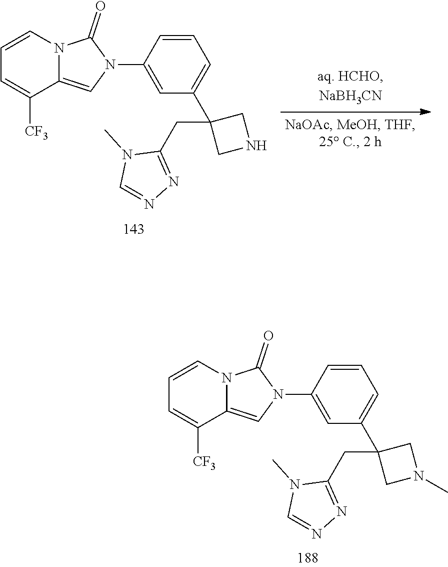 [SMILES: Cn1cnnc1CC1(c2cccc(-n3cc4c(C(F)(F)F)cccn4c3=O)c2)CNC1 ; CN1CC(Cc2nncn2C)(c2cccc(-n3cc4c(C(F)(F)F)cccn4c3=O)c2)C1]

### Synthesis of 188

To a solution of 143 (0.47 g, 1.10 mmol) in THF (10 mL) and MeOH (10 mL) was added HCHO (238 uL, 3.29 mmol, 38% purity), sodium acetate (179 mg, 2.19 mmol) and sodium cyanoborohydride (68.9 mg, 1.10 mmol). The mixture was stirred at 25° C. for 2 h. The mixture was adjusted to pH=8 with saturated aqueous sodium bicarbonate solution (20 ml) and extracted with DCM (5×10 ml). The combined organic layers were dried over anhydrous sodium sulfate, filtered, and the filtrate was concentrated under reduced pressure. The residue was purified by reverse phase HPLC to afford Compound 188 (15 mg, 2% yield) as a white solid.

LCMS: (ES, m/z): [M+H]⁺ 443. H-NMR: (400 MHz, CD₃OD-d₄, ppm): δ 8.27 (s, 1H), 7.78 (d, J=7.6 Hz, 1H), 7.70 (d, J=8.0 Hz, 1H), 7.56-7.45 (m, 2H), 7.14 (s, 1H), 7.07-7.03 (m, 2H), 6.37 (t, J=7.2 Hz, 1H), 4.61 (d, J=9.6 Hz, 2H), 4.36 (d, J=9.6 Hz, 2H), 3.57 (s, 2H), 3.02 (s, 3H), 2.87 (s, 3H)

### Example 189. Synthesis of Compound 189

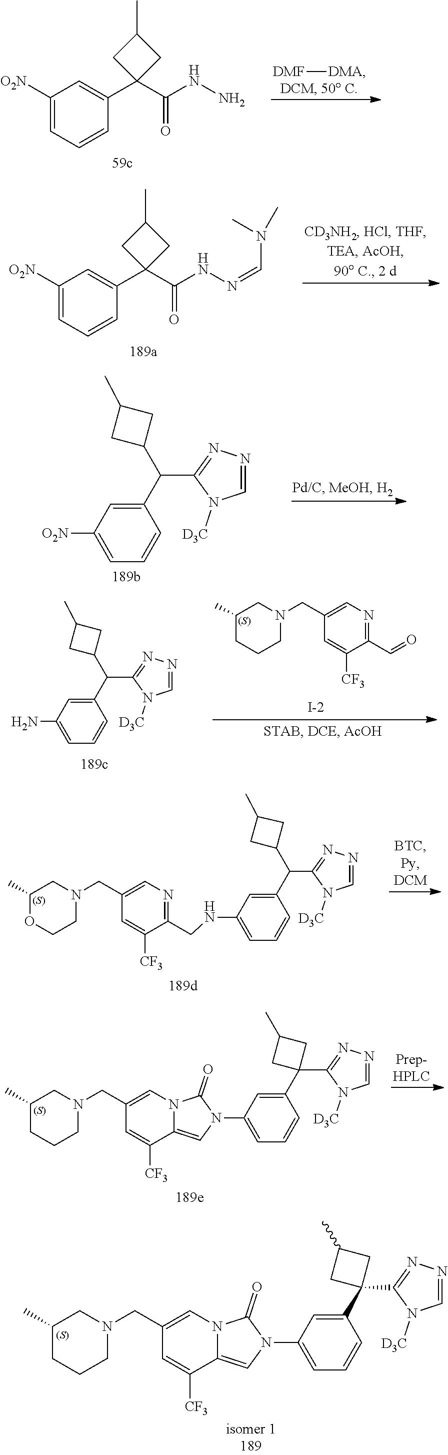

### Synthesis of 189a

To a stirred solution of 59c (5 g, 20.059 mmol, 1 equiv) in DCM (100 mL) were added DMF-DMA (1195 g, 100.295 mmol, 5 equiv) at room temperature under nitrogen atmosphere. The resulting mixture was stirred overnight at 50° C. The resulting mixture was concentrated under vacuum. The residue was purified by silica gel column chromatography, eluted with CH₂Cl₂/MeOH (30:1) to afford 189a (5 g, 76.17%) as a yellow solid.

### Synthesis of 189b

Into a 250 mL 3-necked round-bottom flask were added CD₃NH₂·HCl (2.90 g, 41,070 mmol, 5 equiv), THE (50 ml), TEA (4.24 g, 41,891 mmol, 5.1 equiv), 189a (2.5 g, 8.214 mmol, 1 equiv) and AcOH (0.49 g, 8.214 mmol, 1 equiv) at room temperature. The resulting mixture was stirred for 2 days at 90° C. The resulting mixture was diluted with water (100 ml). The aqueous layer was extracted with EtOAc (3×100 mL). The organic layers were concentrated under reduced pressure. The residue was purified by Prep-TLC (DCM/MeOH 12:1) to afford 189b (680 mg, 27.36%) as a Brown yellow solid.

### Synthesis of 189c

To a solution of 189b (630 mg, 2.288 mmol, 1 equiv) in MeOH (20 mL) was added Pd/C (80 mg, 12%) under nitrogen atmosphere in a 100 mL round-bottom flask. The mixture was hydrogenated at room temperature for 3h under hydrogen atmosphere using a hydrogen balloon, filtered through a Celite pad, and concentrated under reduced pressure. This resulted in 189c (500 mg, 81.94%) as an off-white solid. The crude product was used in the next step directly without further purification.

### Synthesis of 189d

To a stirred solution of 189c (480 mg, 1.956 mmol, 1 equiv) and 1-2 (560.12 mg, 1.956 mmol, 1 equiv) in DCE (5 mL) were added STAB (829.30 mg, 3.912 mmol, 2 equiv) and AcOH (117.49 mg, 1.956 mmol 1 equiv) at room temperature under nitrogen atmosphere. The reaction was quenched with saturated NH₄Cl (aq.) at room temperature. The aqueous layer was extracted with DCM (2×50 mL). The residue was purified by Prep-TLC (DCM/MeOH 15:1) to afford 189d (530 mg, 49.91%) as a white solid,

### Synthesis of 189e

To a stirred solution of 189d (510 mg, 0.989 mmol, 1 equiv) and pyridine (782.34 mg, 9.890 mmol, 10 equiv) in DCM (10 mL) were added triphosgene (117.40 mg, 0.396 mmol, 0.4 equiv) at room temperature under nitrogen atmosphere. The resulting mixture was stirred for 10 min at room temperature. The reaction was quenched with saturated NaHCO₃ (aq.) at room temperature. The aqueous layer was extracted with DCM (2×50 mL). The organic layers were concentrated under reduced pressure. The residue was purified by Prep-TLC (DCM/MeOH 15:1) to afford 189e (400 mg, 67.20%) as a yellow solid.

### Synthesis of 189

The 189e (400 mg, 0.739 mmol, 1 equiv) was purified by Prep-HPLC with the following conditions (Column: Kinetex EVO C18 Column, 30*150, 5 um; Mobile Phase A: Water (10 mmol/L NH₄HCO₃), Mobile Phase B: ACN; Flow rate: 60 mL/min; Gradient: 40% B to 60% B in 8 min, 60% B; Wave Length: 220 nm; RT1 (min): 7.37) to afford Compound 189 (143.3 mg, 35.47%) as a yellow solid.

LC-MS: (ES, m/z): [M+H]⁺ 542. H-NMR: (400 MHz, DMSO-d₆, 3 ppm): 0.81-0.93 (m, 4H), 1.02-1.13 (d, 3H), 1.43-1.46 (m, 1H), 1.57-1.66 (m, 4H), 1.86-1.91 (m, 5H), 2.51-2.56 (m, 3H), 2.72-2.77 (m, 2H), 2.81-2.93 (m, 2H), 3.24 (s, 2H), 7.00 (s, 1H), 7.24-7.26 (d, 1H), 7.37 (s, 1H), 7.47-7.51 (m, 1H), 7.63-7.65 (m, 2H), 7.84 (s, 1H), 8.29 (s, 1H).

### Example 190. Synthesis of Compound 190

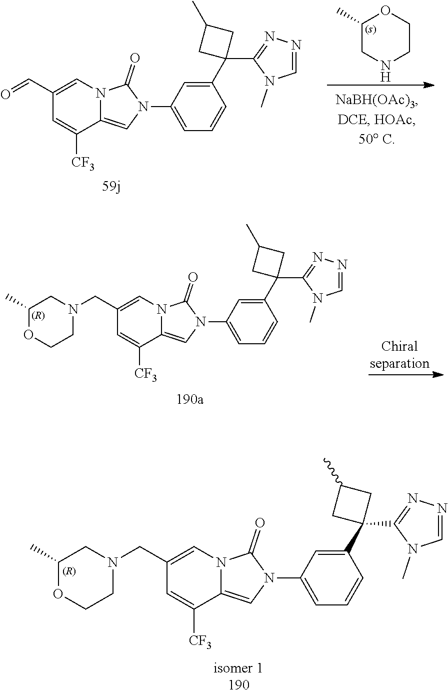

### Synthesis of 190a

Into a 50 mL 3-necked round-bottom flask were added 59j (700 mg, 1.537 mmol, 1 equiv), (2S)-2-methylmorpholine (310.93 mg, 3.074 mmol, 2 equiv), DCE (15 mL) and HOAc (184.60 mg, 3,074 mmol, 2 equiv) at room temperature. The resulting mixture was stirred for 30 min at room temperature under nitrogen atmosphere. To the above mixture was added NaBH(OAc)₃ (651.49 mg, 3.074 mmol, 2 equiv) at room temperature. The resulting mixture was stirred overnight at room temperature. The reaction was quenched with sat. NH₄Cl (aq.) (10 ml) at room temperature. The resulting mixture was extracted with CH₂Cl₂ and MeOH (10:1) (5×10 mL). The combined organic layers were concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 16% B to 27% B in 8 min; Wave Length: 254; 220 nm; RT1 (min): 7.85) to afford 190a (350 mg, 42.12%) as a yellow solid.

### Synthesis of 190

The crude 190a (350 mg) was purified by Chiral separation with the following conditions (Column: CHIRALPAK IC, 2*25 cm, 5 am; Mobile Phase A: Hex (0.5% 2M NH₃ MeOH)-HPLC, Mobile Phase B: EtOH:DCM=1:1-HPLC; Flow rate: 20 mL/min; Gradient: 50% B to 65% B in 20 min; Wave Length: 220/254 nm; RT1 (min): 8.96; RT2 (min): 13.78; The second peak was the product; Sample Solvent: EtOH:DCM=1:1-HPLC; Injection Volume: 2 mL; Number Of Runs: 6) to afford crude product (150 mg). The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 29% B to 55% B in 8 min; Wave Length: 220 nm; RT1 (min): 7.73) to afford product. The product was dissolved in THF (5 mL) and isolute Si-thiol (3 mg) was added. The mixture was stirred for 30 min. Then the solution was filtered. After concentrated the filtrate, this resulted in Compound 190 (42.8 mg, 12.23%) as a yellow solid.

LC-MS: (ES, m/z): [M+H]⁺ 541. H-NMR: (400 MHz, CD₃OD, ppm): δ 1.14-1.18 (m, 6H), δ 1.86-1.91 (t, 1H), δ 2.19-2.20 (m, 1H), δ 2.41-2.55 (m, 2H), δ 2.74-2.83 (m, 2H), δ 3.14-3.19 (m, 2H), δ 3.32-3.37 (m, 5H), δ 3.66-3.70 (m, 2H), δ 3.84-3.85 (m, 1H), δ 7.11-7.15 (dd, 2H), δ 7.21-7.23 (m, 1H), δ 7.51-7.59 (m, 2H), δ 7.69 (s, 2H), δ 8.39 (s, 1H).

### Example 191. Synthesis of Compound 191

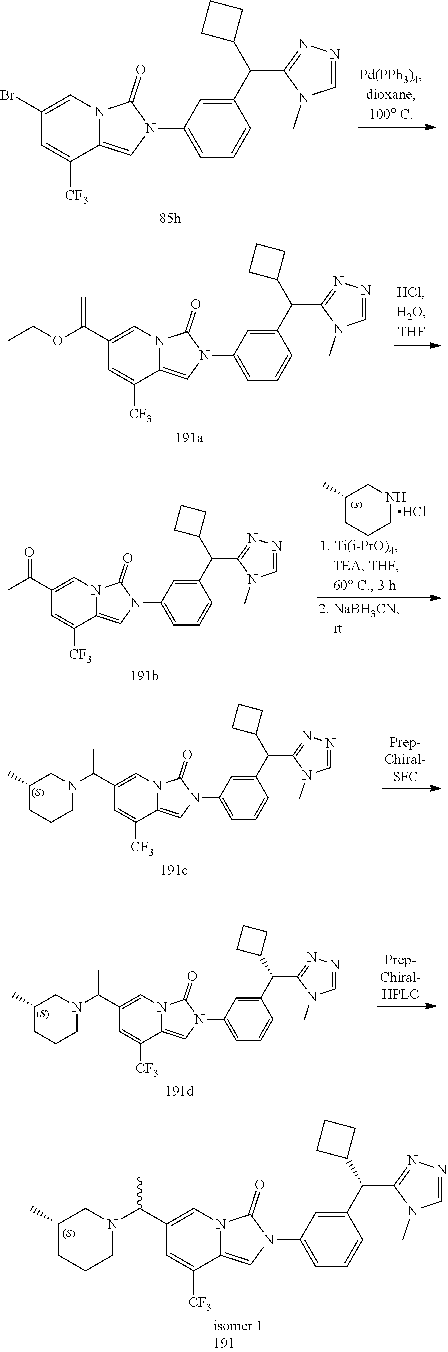

### Synthesis of 191a

To a stirred mixture of 85h (15 g, 29.625 mmol, 1 equiv) and tributyl (I-ethoxyethenyl) stannane (16.05 g, 44.438 mmol, 1.5 equiv) in dioxane (150 mL) was added Pd(PPh₃)4 (3.42 g, 2.963 mmol, 0.1 equiv) at room temperature. The resulting mixture was stirred overnight at 100° C. under nitrogen atmosphere. The mixture was allowed to cool down to room temperature. The reaction was quenched by the addition of saturated NH₄Cl (aq.) (3 mL) at room temperature. The aqueous layer was extracted with CH₂Cl₂/MeOH (10:1) (3×100 mL). The resulting mixture was concentrated under reduced pressure. The residue was purified by silica gel column chromatography, eluted with CH₂Cl₂/MeOH (80:1) to afford 191a (11.5 g, 70.22%) as a yellow solid.

### Synthesis of 191b

To a stirred mixture of 191a (11.5 g, 23.115 mmol, 1 equiv) in THE (115 mL) was added HCl (115 mL, 1M) at room temperature. The resulting mixture was stirred overnight at room temperature. The resulting mixture was diluted with water (230 mL). The aqueous layer was extracted with CH₂Cl₂/Me-H (10:1) (3×200 mL). The resulting mixture was concentrated under reduced pressure to afford 191 b (10.2 g, 89.30%) as a yellow solid.

### Synthesis of 191c

To a stirred mixture of 191b (2.5 g, 5.325 mmol, 1 equiv) and (3S)-3-methylpiperidine hydrochloride (2.17 g, 15,975 mmol, 3 equiv) in TH-F (25 mL) were added TEA (5.39 g, 53.250 mmol, 10 equiv) and Ti(Oi-Pr)4 (6.05 g, 21.300 mmol, 4 equiv) at room temperature. The resulting mixture was stirred for 3h at 60° C. under air atmosphere. To the above mixture was added NaBH₃CN (1.00 g, 15,975 mmol, 3 equiv) at room temperature. The resulting mixture was stirred overnight at room temperature. The reaction was quenched by the addition of saturated NH₄Cl (aq.) (50 mL) at room temperature. The aqueous layer was extracted with CH₂Cl₂/MeOH (10:1) (3×80 mL). The resulting mixture was concentrated under reduced pressure. The residue was purified by silica gel column chromatography, eluted with CH₂Cl₂/MeOH (60:1) to afford 191c (1.2g). The residue was purified by Prep-TLC (CH₂Cl₂/MeOH 12:1) to afford 191c (950 mg). The residue was purified by reverse flash chromatography with the following conditions: column, silica gel; mobile phase, Mobile Phase A: Water (10 mmol/L NH₄HCO₃), Mobile Phase B: ACN, 2% to 100% gradient in 40 min; detector, UV 254 nm to afford 191c (740 mg, 24.39%) as a yellow solid.

### Synthesis of 191d

The 191c (740 mg) was purified by SFC with the following conditions (Column: CHIRAL ART Cellulose-SB, 3*25 cm, 5 urn; Mobile Phase A: CO₂, Mobile Phase B: MeOH (0.1% DEA); Flow rate: 70 mL/min; Gradient: isocratic 35% B; Column Temperature (° C.): 35; Back Pressure (bar): 100; Wave Length: 220 nm; RT1 (min): 5.48, RT1 (min): 8.72, the first peak is product) to afford 191d (340 mg) as a yellow solid.

### Synthesis of 191

The 191d (340 mg) was purified by chiral separation with the following conditions (Column: CHIRALPAK IF, 2*25 cm, 5 μm; Mobile Phase A: Hex (0.5% 2M NH-3-MeOH), Mobile Phase B: EtOH:DCM=1:1; Flow rate: 20 mL/min; Gradient: 20% B to 20% B in 13.5 min; Wave Length: 220/254 am; RT (min): 10.50; RT2 (min): 12.00; the first peak is product) to afford Compound 191 (100.2 mg, 29.78%) as a yellow solid,

LCMS: (ES, m/z): [M+H]⁺ 553. H-NMR: H NMR (400 MHz, DMSO, ppm): δ 0.70-0.90 (m, 4H), δ 1.25 (s, 3H), δ 1.30-1.50 (m, 2H), δ 1.61-1.89 (m, 8H), δ 1.91-1.95 (m, 1H), δ 2.08-2.10 (m, 1H), δ 2.67-2.75 (m, 2H), δ 3.18-3.26 (m, 1H), δ 3.34-3.44 (m, 4H), δ 4.24-4.28 (d, 1H), δ 7.08 (s, 1H), δ 7.18-7.21 (d, 1H), δ 7.31 (s, 1H), δ 7.42-7.47 (m, 1H), δ 7.56 (s, 1H), δ 7.68-7.74 (m, 2H), δ 8.33 (s, 1H).

### Example 192. Synthesis of Compound 192

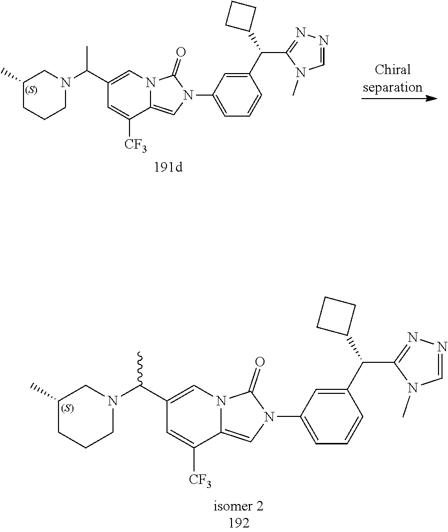

### Synthesis of 192

The 191d (340 mg) was purified by chiral separation with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min; Gradient: 20% B to 20% B in 13.5 min; Wave Length: 220/254 nm; RT1 (min): 10.50; RT2 (min): 12.00; the second peak is product) to afford Compound 192 (95.8 mg, 27.39%) as a yellow solid.

LCMS: (ES, m/z): [M+H]⁺ 553. H-NMR: H NMR (400 MHz, DMSO, ppm): δ 0.75-0.90 (m, 4H), δ 1.25 (s, 3H), δ 1.41-1.81 (m, 10H), δ 2.01-2.11 (m, 2H), δ 2.67-2.73 (m, 2H), δ 3.18-3.30 (m, 1H), δ 3.34-3.44 (m, 4H), δ 4.25-4.28 (d, 1H), δ 7.08 (s, 1H), δ 7.18-7.21 (d, 1H), δ 7.31 (s, 1H), δ 7.42-7.47 (m, 1H), δ 7.58 (s, 1H), δ 7.67-7.70 (m, 1H), δ 7.73 (s, 1H), δ 8.33 (s, 1H).

### Example 193. Synthesis of Compound 193

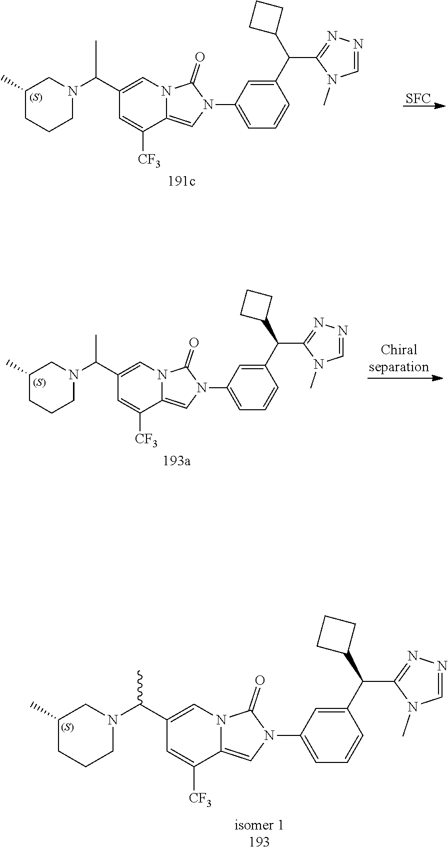

### Synthesis of 193a

The 191c (740 m-g) was purified by Prep-SFC with the following conditions (Column: CHIRAL ART Cellulose-SB, 3*25 cm, 5 am; Mobile Phase A: CO₂, Mobile Phase B: MeOH (0.1% DEA); Flow rate: 70 mL/min; Gradient: isocratic 35% B; Column Temperature (° C.): 35; Back Pressure (bar): 100; Wave Length: 220 nm; RT1 (min): 5.48, RT1 (min): 8.72, the second peak is product) to afford 193a (320 mg) as a yellow solid.

### Synthesis of 193

The 193a (320 mg) was purified by chiral separation with the following conditions (Column: (R, R)-WHELK-O1-Kromasi, 5*25 cm, 5 μm; Mobile Phase A: Hex (0-5% 2M NH₃-MeOH), Mobile Phase B: EtOH:DCM=1:1; Flow rate: 20 mL/min; Gradient: 50% B to 50% B in 11 min; Wave Length: 220/254 nm; RT1 (min): 7.03; RT2 (min): 8.52; the first product is product) to afford Compound 193 (92.9 mg, 28.74%) as a yellow solid.

LCMS: (ES, m/z): [M+H]⁺ 553

H-NMR: H NMR (400 MHz, DMSO, ppm): δ 0.80-0.98 (m, 4H), δ 1.23 (s, 3H), δ 1.40-1.50 (m, 1H), δ 1.55-1.81 (m, 9H), δ 2.01-2.10 (m, 2H), δ 2.67-2.75 (m, 2H), δ 3.16-3.26 (m, 1H), δ 3.39-3.47 (m, 4H), δ 4.19-4.30 (d, 1H), δ 7.08 (s, 1H), δ 7.18-7.21 (d, 1H), δ 7.31 (s, 1H), δ 7.42-7.47 (m, 1H), δ 7.59 (s, 1H), δ 7.67-7.70 (m, 1H), δ 7.73 (s, 1H); δ 8.33 (s, 1H).

Alternatively, Compound 193 may be also prepared in the manner outlined below:

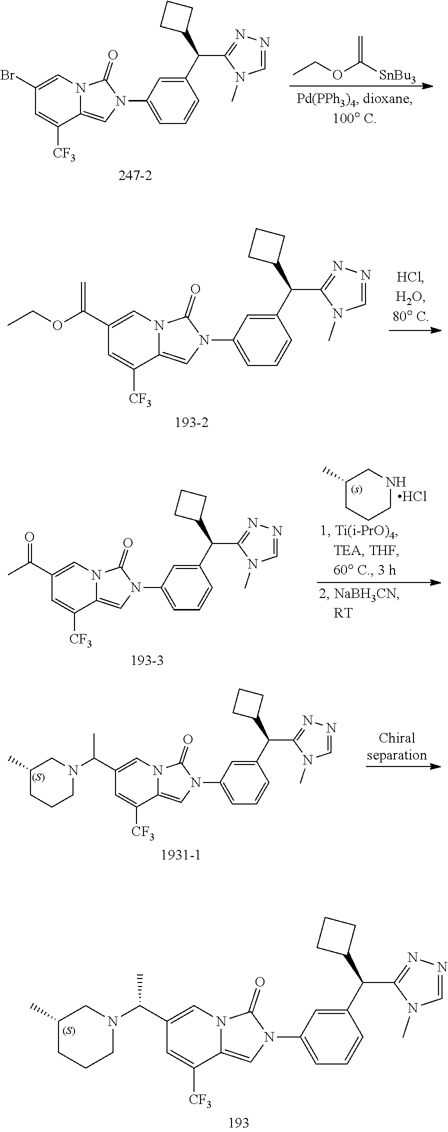

### 1. Synthesis of 193-2

To a stirred solution of 247-2 (30 g, 59.250 mmol, 1 equiv) and tributyl(1-ethoxyethenyl)stannane (32.10 g, 88,875 mmol, 1.5 equiv) in dioxane (400 mL) were added Pd(PPh3)4 (6.85 g, 5,925 mmol, 0.1 equiv) at room temperature. The resulting mixture was stirred overnight at 100° C. under nitrogen atmosphere. The reaction was quenched with water (800 mL) at room temperature. The resulting mixture was extracted with CH2Cl2 (3×500 mL). The resulting mixture was concentrated under reduced pressure. The residue was purified by silica gel column chromatography, eluted with CH2Cl2/MeOH (50:1) to afford 193-2 (22 g, 70.90%) as a yellow solid.

### 2. Synthesis of 193-3

A solution of 193-2 (55 g, 110.548 mmol, 1 equiv) in HCl (1 M 600 mL) was stirred overnight at 80° C. The mixture was neutralized to pH 7 with saturated NaHCO₃ (aq.). The resulting mixture was extracted with CH2Cl2 (3×1000 mL). The resulting mixture was concentrated under reduced pressure. The residue was purified by silica gel column chromatography, eluted with CH2Cl2/MeOH (30:1) to afford 193-3 (20 g, 37.00%) as a yellow solid.

### 3. Synthesis of 193-1

To a stirred solution of 193-3 (17 g, 36.211 mmol, 1.00 equiv) and (3S)-3-methylpiperidine hydrochloride (14.74 g, 108.633 mmol, 3 equiv) in THF (300 mL) were added Ti(Oi-Pr)4 (41.17 g, 144.844 mmol, 4 equiv) and TEA (36.64 g, 362.110 mmol, 10 equiv) at room temperature. The resulting mixture was stirred for 3h at 60° C. To the above mixture was added NaBH3CN (6.83 g, 108.633 mmol, 3 equiv) at room temperature. The resulting mixture was stirred overnight at room temperature. The reaction was quenched with NH4Cl (aq.) (500 mL) at room temperature. The resulting mixture was extracted with CH2Cl2 (3×800 mL). The resulting mixture was concentrated under reduced pressure. The residue was purified by reverse flash chromatography with the following conditions: column, C18 silica gel; mobile phase, MeCN in Water (1.0 mmol/NH4HCO3), 30% to 55% gradient in 20 min; detector, UV 254 un. This resulted in 193-1 (6.5 g, 32.16%) as a yellow solid.

### 4. Synthesis of 193-0

The 193-1 (6.5 g) was purified by Prep-SFC with the following conditions (Column: (R, R)-WHELK-O1-Kromasil, 5*25 cm, 5 um; Mobile Phase A: C02, Mobile Phase B: IPA (0.5% 2M NH3-MeOH); Flow rate: 200 mL/min; Gradient: isocratic 60% B; Column Temperature (° C.):35; Back Pressure (bar): 100; Wave Length: 220 nm; RT1 (min): 9.2; RT2 (min): 12.83; the first product is product) to afford 193 (2.1913g, 33.54%) as a yellow solid.

LCMS-193: (ES, m/z): [M+H]⁺ 553

H-NMR-193: H NMR (400 MHz, DMSO, ppm): δ 0.80-0.98 (m, 4H), δ 1.23-1.32 (m, 3H), δ 1.40-1.50 (m, 1H), δ 1.45-1.69 (m, 4H), δ 1.70-1.85 (m, 5H), δ 2.01-2.10 (m, 2H), δ 2.67-2.75 (m, 2H), δ 3.16-3.26 (m, 1H), δ 3.39-3.47 (m, 4H), δ 4.19-4.30 (d, 1H), δ 7.08 (s, 1H), δ 7.18-7.21 (d, 1H), δ 7.31 (s, 1H), δ 7.42-7.47 (m, 1H), δ 7.59 (s, 1H), δ 7.67-7.70 (m, 1H), δ 7.73 (s, 1H); δ 8.33 (s, 1H).

### Example 194. Synthesis of Compound 194

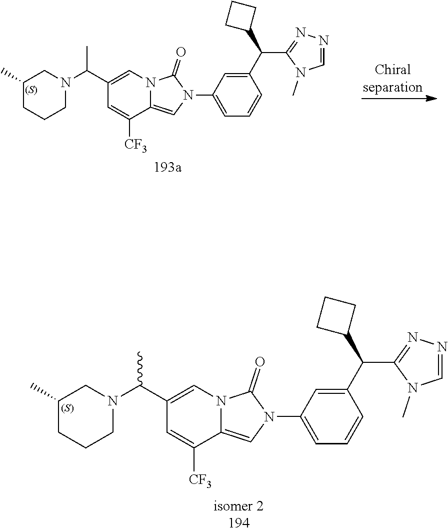

### Synthesis of 194

193a (320 mg) was purified by chiral separation with the following conditions (Column: (R, R)-WHELK-O01-Kromasi, 5*25 cm, 5 μm; Mobile Phase A: Hex (0.5% 2M NH₃-MeOH), Mobile Phase B: EtOH:DCM=1:1; Flow rate: 20 mL/min; Gradient: 50% B to 50% B in 11 min; Wave Length: 220/254 nm; RT1 (min): 7.03; RT2 (min): 8.52; the second product is product) to afford Compound 194 (92.1 mg, 28.46%) as a yellow solid.

LCMS: (ES, m/z): [M+H]⁺ 553

H-NMR: H NMR (400 MHz, DMSO, ppm): δ 0.80-0.93 (m, 4H), δ 1.21-1.31 (d, 3H), δ 1.34-1.44 (m, 1H), δ 1.45-1.56 (m, 3H), δ 1.57-1.82 (m, 6H), δ 1.85-2.10 (m, 2H), δ 2.67-2.75 (m, 2H), δ 3.20-3.26 (m, 1H), δ 3.34-3.44 (m, 4H), δ 4.25-4.28 (d, 1H), δ 7.08 (s, 1H), δ 7.18-7.21 (m, 1H), δ 7.31 (s, 1H), δ 7.42-7.47 (m, 1H), δ 7.58 (s, 1H), δ 7.67-7.70 (m, 1H), δ 7.73 (s, 1H); δ 8.33 (s, 1H).

### Example 195. Synthesis of Compound 195

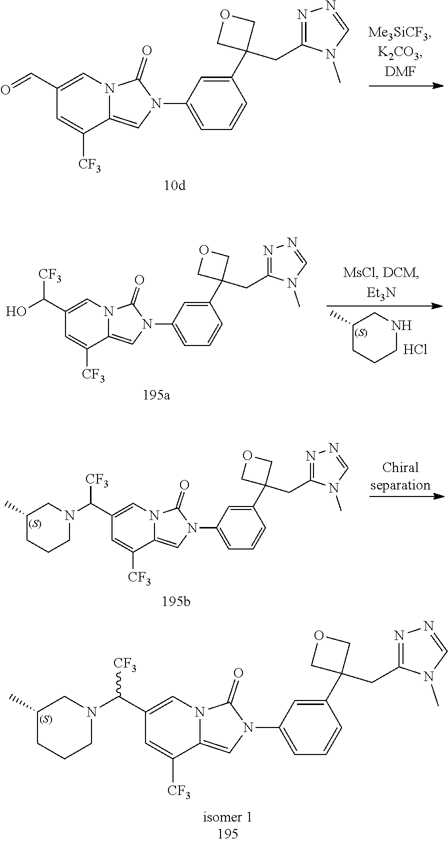

### Synthesis of 195a

To a stirred solution of 10d (1 g, 2,186 mmol, 1 equiv) and TMSCF₃ (0.62 g, 4,372 mmol, 2 equiv) in DMF (10 mL) was added K₂CO₃ (0.03 g, 0.219 mmol, 0.1 equiv) at room temperature. The resulting mixture was stirred for 2 h at room temperature. The reaction solution was purified by reverse flash chromatography with the following conditions: column, C18 silica gel; mobile phase, MeCN in water (10 mmoL/L NH₄HCO₃), 10% to 65% gradient in 40 min; detector, UV 254 nm. This resulted in 195a (600 mg, 52.04%) as a yellow solid.

### Synthesis of 195b

To a stirred solution of 195a (600 mg, 1.138 mmol, 1 equiv) and Et₃N (575.57 mg, 5.690 mmol, 5 equiv) in DCM (15 mL) was added MsCl (195.47 mg, 1.707 mmol, 15 equiv) dropwise at 0° C. The resulting mixture was stirred for 3 h at 0° C. To the above mixture was added (3S)-3-methylpiperidine hydrochloride (462.91 mg, 3.414 mmol, 3 equiv). The resulting mixture was stirred for additional 4 h at room temperature. The reaction was quenched by the addition of water (20 mL). The resulting mixture was extracted with CH₂Cl₂/MeOH=10/1 (3×25 mL). The combined organic layers were concentrated under reduced pressure. The residue was purified by Prep-TLC (CH₂Cl₂/MeOH 15:1) to afford 195b (200 mg, 28.89%) as a yellow solid.

### Synthesis of 195

195b (200 mg, 0.329 mmol, 1 equiv) (200 mg) was purified by Prep-Chiral-HPLC with the following conditions (Column: CHIRALPAK IC, 2*25 cm, 5 um; Mobile Phase A: MtBE (0.5% 2M NH3-MeOH), Mobile Phase B: MeOH:DCM=1:1; Flow rate: 20 mL/min; Gradient: 20% B to 20% B in 35 min; Wave Length: 220/254 nm; RT1 ((min): 29.32) to afford crude product (44.2 mg). The crude product (44.2 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 8 min, Wave Length: 220 nm; RT1 (min): 7.42) to afford Compound 195 (22.3 mg, 11.15%) as a yellow solid.

LC-MS: (ES, m/z): [M+H]⁺ 609. H-NMR: (400 MHz, DMSO-d6, ppm): δ 0.81-0.90 (m, 4H), δ 1.38-1.64 (m, 4H), δ 1.94-2.27 (m, 2H), δ 2.72-2.94 (m, 5H), δ 3.53 (s, 2H), δ 4.56-4.65 (m, 1H), δ 4.90-4.96 (m, 4H), δ 6.89-6.96 (m, 2H), δ 7.38-7.44 (m, 3H), δ 7.72-7.80 (m, 2H), δ 8.20 (s, 1H).

### Example 196. Synthesis of Compound 196

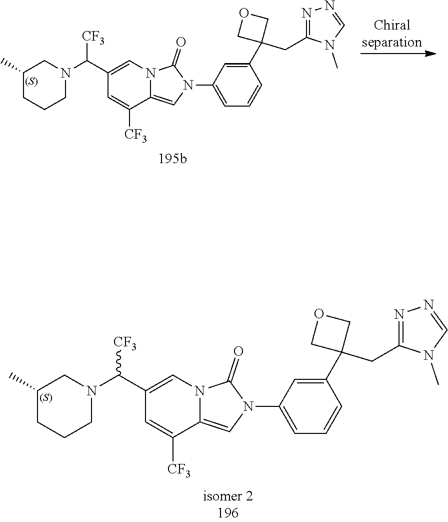

### Synthesis of 196

195b (200 mg, 0.329 mmol, 1 equiv) (200 mg) was purified by Prep-Chiral-HPLC with the following conditions (Column: CHIRALPAK IC, 2*25 cm, 5 L m; Mobile Phase A: MtBE (0.5% 2M NH₃-MeOH), Mobile Phase B: MeOH:DCM=1:1; Flow rate: 20 mL/min; Gradient: 20% B to 20% B in 35 min; Wave Length: 220/254 nm; RT2 (min): 33.20) to Compound 196 (44.2 mg, 22.10%) as a yellow solid.

LC-MS: (ES, m/z): [M+H]⁺ 609. H-NMR: (400 MHz, DMSO-d6, ppm): δ 0.82-088 (m, 4H), δ 1.32-1.40 (m, 1H), δ 1.42-1.65 (m, 3H), δ 1.93-1.98 (m, 1H), δ 2.26-2.34 (m, 1H), δ 2.83-2.92 (m, 2H), δ 2.97 (s, 3H), δ 3.53 (s, 2H), δ 4.62-4.64 (m, 1H), δ 4.91-4.97 (m, 4H), δ 6.90-6.92 (m, 2H), δ 6.98 (s, 1H), δ 7.39-7.43 (m, 3H), δ 7.74-7.77 (m, 2H), δ 8.20 (s, 1H).

### Example 197. Synthesis of Compound 197

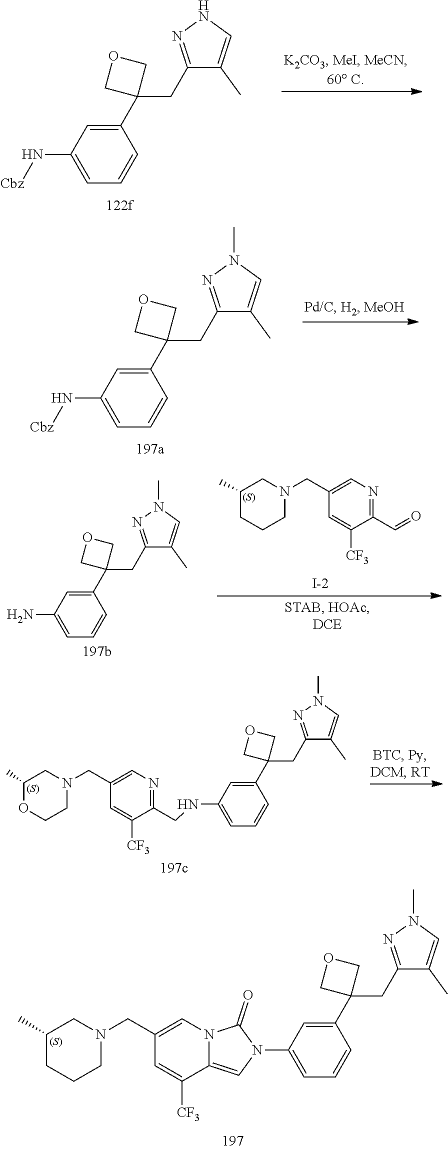

### Synthesis of 197a

To a stirred mixture of 122f (650 mg, 1.722 mmol, 1 equiv) and K₂CO₃ (714.02 mg, 5.166 mmol, 3.00 equiv) in MeCN (10 mL-1) were added Mel (488.87 mg, 3.444 mmol, 2 equiv) at room temperature under nitrogen atmosphere. The resulting Mixture was stirred overnight at 60° C. The resulting mixture was dilated with water (100 mL). The aqueous layer was extracted with EtOAc (2×50 mL). The organic layers were concentrated under reduced pressure. The residue was purified by Prep-TLC (DCM/MeOH 30:1) to afford 197a (300 mg, 42.28%) as a white solid.

### Synthesis of 197b

To a solution of 197a (360 mg, 0.920 mmol, 1 equiv) in.MeOH (10 mL) was added Pd/C (40 mg, 10%) under nitrogen atmosphere in a 100 ml round-bottom flask. The mixture was hydrogenated at room temperature for 3h under hydrogen atmosphere using a hydrogen balloon, filtered through a Celite pad, and concentrated under reduced pressure. This resulted in 197b-(240 mg, 91.27%) as a light yellow oil. The crude product was used in the next step directly without further purification.

### Synthesis of 197c

To a stirred solution of 197b (120 mg, 0.466 mmol, 1 equiv) and I-2 (133.50 mg, 0.466 mmol, 1 equiv) in DCE (5 mL) were added STAB (197.66 mg, 0.932 mmol, 2 equiv) and HOAc (28.00 mg, 0.466 mmol, 1 equiv) at room temperature under nitrogen atmosphere. The resulting mixture was stirred for 3h at room temperature. The reaction was quenched with saturated NH₄Cl (aq.) at room temperature. The aqueous layer was extracted with DCM (2×20 mL). The organic layers were concentrated under reduced pressure. The residue was purified by Prep-TLC (DCM/MeOH 15:1) to afford 197c (180 mg, 68.04%) as an off-white solid.

### Synthesis of 197

To a stirred solution of 197c (180 mg, 0.341 mmol, 1 equiv) and pyridine (269.84 mg, 3,410 mmol, 10 equiv) in DCM (10 mL) was added triphosgene (40.49 mg 0.136 mmol, 0.4 equiv) at room temperature. The resulting mixture was stirred for 10 min at room temperature. The reaction was quenched with saturated NaHCO₃ (aq.) at room temperature. The aqueous layer was extracted with DCM (220 mL). The organic layers were concentrated under reduced pressure. The residue was purified by reverse flash chromatography with the following conditions: column, C18 silica gel; Mobile Phase A: Water (10 mmol/L NH₄HCO₃), Mobile Phase B: ACN; 10% B to 60% B gradient in 15 min; detector, UV 254 nm. This resulted in Compound 197 (99.3 mg, 52.31%) as a yellow solid.

LC-MS: (ES, m/z): [M+H]⁺ 554

H-NMR: (400 MHz, DMSO-d₆, 3 ppm): 0.82-0.89 (m, 4H), 1.23 (s, 3H), 1.35-1.72 (m, 5H), 1.84-1.93 (m, 1H), 2.67-2.77 (m, 2H), 3.16 (s, 2H), 3.24 (s, 2H), 3.70 (s, 3H), 4.82-4.90 (m, 4H), 6.87-6.89 (d, 1H), 7.00 (s, 1H), 7.16 (s, 1H), 7.19 (s, 1H), 7.24 (s, 1H), 7.37-7.39 (m, 1H), 7.65 (s, 1H), 7.72-7.74 (d, 1H).

### Example 198. Synthesis of Compound 198

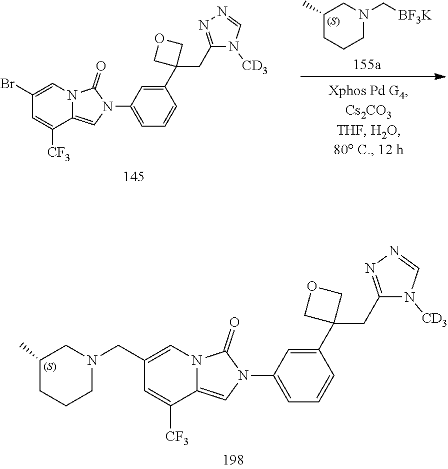

### Synthesis of 198

To a solution of 155a (205 mg, 938 umol) and 145 (240 mg, 469 umol) in THF (6 mL) and water (1.5 mL) was added cesium carbonate (458 mg, 1.41 mmol), dicyclohexyl-[2-(2,4,6-triisopropylphenyl)phenyl]phosphane;methanesulfonate;[2-[2-(methylamino)phenyl]phenyl]palladium(1+) (40.3 mg, 46.9 umol). The mixture was stirred at 80° C. for 12 h under N₂. The mixture was washed with water (30 ml) and extracted with DCM (3×20 ml). The combined organic layers were dried over anhydrous sodium sulfate, filtered and the filtrate was concentrated under reduced pressure. The residue was purified by reverse phase HPLC to afford Compound 198 (50 mg, 19% yield) as a yellow solid.

LCMS: (ES, m/z): [M+H]⁺ 544. H-NMR: (400 MHz, DMSO-d₆, ppm): δ 8.21 (s, 1H), 8.16 (s, 1H), 7.80-7.73 (m, 1H), 7.67 (s, 1H), 7.44-7.39 (m, 2H), 7.33 (s, 1H), 7.02 (s, 1H), 6.90 (d, J=8.0 Hz, 1H), 4.98-4.92 (m, 4H), 3.55 (s, 2H), 3.27 (s, 2H), 2.84-2.71 (m, 2H), 1.94-1.88 (m, 1H), 1.68-1.58 (m, 4H), 1.50-1.43 (m, 1H), 0.84 (d, J=6.0 Hz, 4H)

### Example 199. Synthesis of Compound 199

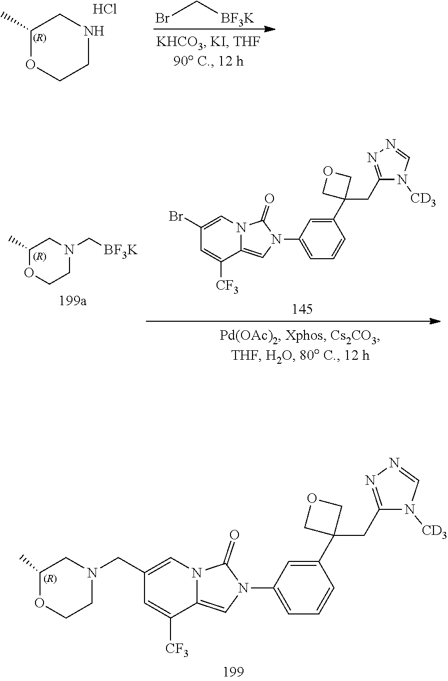

### Synthesis of 199a

To a solution of (2R)-2-methylmorpholine (0.2 g, 1.45 mmol, as a hydrochloride salt), potassium;bromomethyl(trifluoro)boranuide (291 ng, 1.45 mmol), potassium bicarbonate (291 ng, 2.91 mmol) and KI (24.1 mg, 145 umol) in THF (5 mL) was stirred at 90° C. for 12 h under N2. The mixture was concentrated under reduced pressure. The crude product was triturated with acetone (30 ml) at 25° C. for 30 min. Then the mixture was filtered, and the filtrate was concentrated under reduced pressure to afford 199a (250 mg, 77.81% yield) was obtained as a light yellow oil.

### Synthesis of 199

To a solution of 145 (220 mg, 430 umol) and 199a (190 mg, 860 umol) in THF (15 mL) and water (4 mL) was added XPhos (41 mg, 86 umol), cesium carbonate (420 mg, 1.29 mmol) and Pd(OAc)2 (48.3 mg, 215 umol). The mixture was stirred at 80° C. for 12 h. The mixture was washed with water (50 ml) and extracted with EtOAc (3×30 ml), the combined organic layers were dried over anhydrous sodium sulfate, filtered, and the filtrate was concentrated under reduced pressure. The residue was purified by reverse phase HPLC to afford Compound 199 (17 mg, 6.91% yield) as a yellow solid.

LCMS: (ES, m/z): [M+H]⁺ 546. H-NMR: (400 MHz, DMSO-d₆, ppm): δ 8.20 (s, 1H), 7.80-7.72 (m, 1H), 7.69 (s, 1H), 7.45-7.37 (m, 2H), 7.32 (s, 1H), 7.02 (s, 1H), 6.90 (d, J=8.0 Hz, 1H), 4.9-4.87 (m, 4H), 3.75 (d, J=10.0 Hz, 1H), 3.57-3.44 (m, 4H), 3.28 (d, J=4.4 Hz, 2H), 2.74 (d, J=11.2 Hz, 1H), 2.68 (d, J=1.2 Hz, 1H), 2.10-2.00 (m, 1H), 1.74 (t, J=10.8 Hz, 1H), 1.04 (d, J=6.4 Hz, 3H).

### Example 200. Synthesis of Compound 200

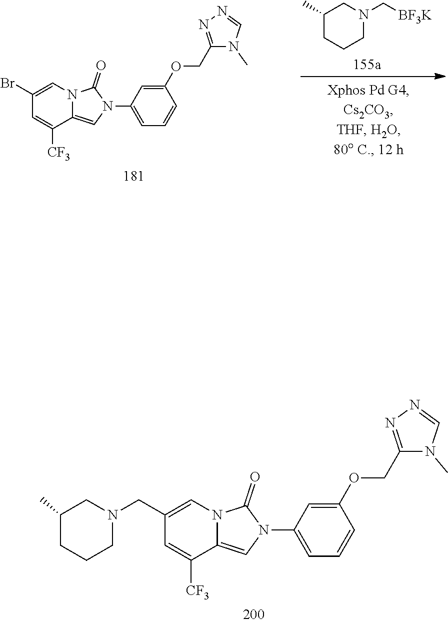

### Synthesis of 200

To a solution of 181 (300 mg, 640 umol) and 155a (280 mg, 1.28 mmol) in THF (4 mL) and water (1 mL) was added cesium carbonate (626 mg, 1.92 mmol), dicyclohexyl-[2-(2,4,6-triisopropylphenyl)phenyl]phosphane;methanesulfonate;[2-[2-(methylamino)phenyl]phenyl] palladium(1+) (55.1 mg, 64.1 umol), the mixture was stirred at 80° C. for 12 hr under N₂. The reaction mixture was diluted with water (10 mL) and extracted with EtOAc (15 mL×3). The combined organic layers were dried over anhydrous sodium sulfate, filtered, and concentrated in vacuum. The residue was purified by reverse phase HPLC to afford Compound 200 (50 mg, 13% yield) as a yellow solid.

LCMS: (ES, m/z): [M+H]⁺ 501. H-NMR: (400 MHz, CD₃OD-d₄, ppm): δ 8.53 (s, 1H), 7.69 (s, 1H), 7.54 (t, J=2.0 Hz, 1H), 7.52-7.46 (m, 1H), 7.40 (br d, J=8.0 Hz, 1H), 7.18 (s, 1H), 7.16-7.12 (m, 2H), 5.43 (s, 2H), 4.62 (br d, J=0.8 Hz, 2H), 3.86 (s, 3H), 2.94-2.82 (m, 2H), 2.04-1.94 (m, 1H), 1.80-1.67 (m, 4H), 1.65-1.58 (m, 1H), 0.91-0.90 (m, 4H)

### Example 201. Synthesis of Compound 201

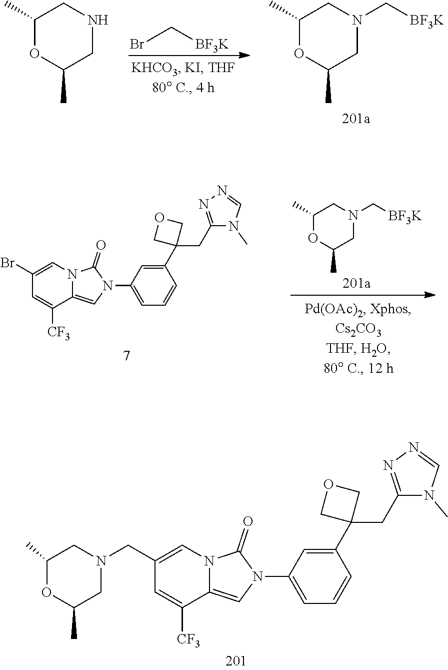

### Synthesis of 201a

To a solution of (2R,6R)-2,6-dimethylmorpholine (0.2 g, 1.74 mmol) and potassium bromomethyl (trifluoro)boronate (348.75 mg, 1.74 mmol) in THF (2 mL) was added KHCO₃ (347.70 mg, 3.47 mmol) and KI (28.83 mg, 173.65 umol). The mixture was stirred at 80° C. for 4 hr. The reaction mixture was evaporated under reduced pressure to give a residue. Then it was poured into acetone (5 ml) and stirred for 20 min at room temperature. The mixture was filtered and the filtrate was evaporated under reduced pressure to get crude product 201a (0.19 g, crude) as colorless oil, which was used into the next step without further purification.

### Synthesis of 201

To a solution of Compound 7 (0.1 g, 196.74 umol) and 201a (138.76 mg, 590.21 umol) in THF (0.5 mL) and 1120 (0.1 mL) was added diacetoxypalladium (4.42 mg, 1967 umol), dicyclohexyl-[2-(2,4,6-triisopropylphenyl)phenyl]phosphane (14.07 mg, 29.51 umol), and cesium carbonate (128.20 mg, 393.48 umol). The mixture was stirred at 100° C. for 12 hr under N₂. The reaction mixture was concentrated under reduced pressure to give a residue. The residue was purified by prep-HPLC (neutral condition; column: Phenomenex C18 80*40 mm*3 um; mobile phase: [water (NH4HCO3)-ACN]; B %: 25%-60%, 8 min). Compound 201 (0,022 g, 19.09% yield) was obtained as a white solid,

LCMS: (ES, m/z): [M+H]⁺ 557. H-NMR: (400 MHz, DMSO-d₆, ppm): δ 8.20 (s, 1H), 7.74 (dd, J=6.8 Hz, J=8.4 Hz, 1H), 7.67 (s, 1H), 7.42-7.38 (m, 2H), 7.31 (s, 1H), 7.03 (s, 1H), 6.89 (d, J=8 Hz, 1H), 4.94 (d, J=6.4 Hz, 2H), 4.91 (d, J=6.0 Hz, 2H), 3.91-3.89 (m, 2H), 3.53 (s, 2H), 3.36-3.21 (m, 2H), 2.96 (s, 3H), 2.50-5.41 (m, 2H), 2.13-2.10 (m, 2H), 1.12 (d, J=6.4 Hz, 6H).

### Example 202. Synthesis of Compound 202

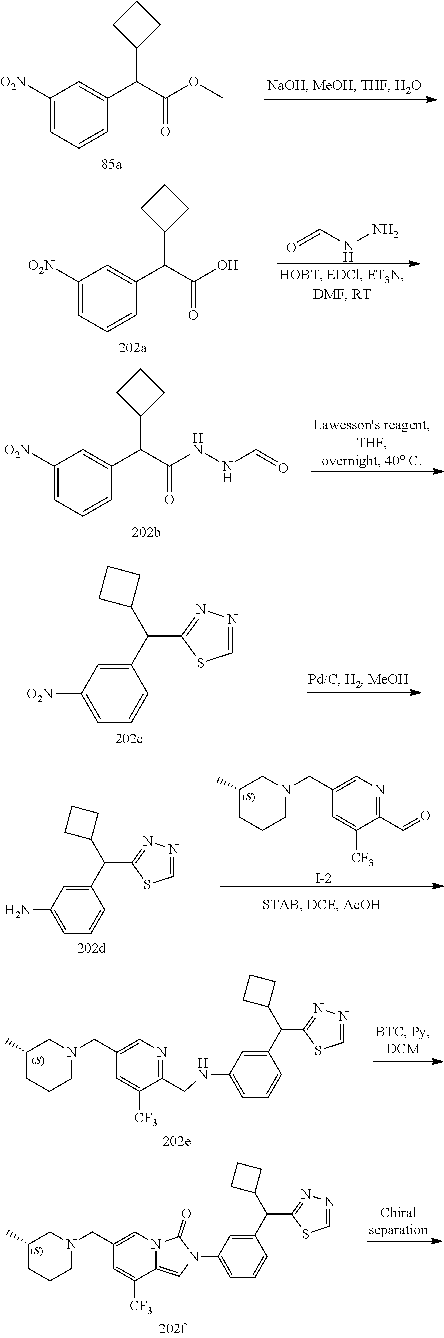

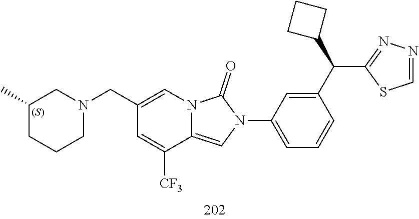

### Synthesis of 202a

Into a 500 mL 3-necked round-bottom flask were added 85a (20 g, 80.236 mmol, 1 equiv), MeOH (200 mL), THF (50 mL), H₂O (50 mL) and NaOH (6.42 g, 160.472 mmol, 2 equiv) at room temperature. The resulting mixture was stirred overnight at room temperature. The mixture was acidified to pH 3 with HCl (aq, 1M). The precipitated solids were collected by filtration and washed with Et₂O (3×30 mL) to afford 202a (17 g, 90.07%) as a white solid.

### Synthesis of 202b

Into a 250 mL 3-necked round-bottom flask were added 202a (8 g, 34.008 mmol, 1 equiv), DMF (80 mL), N-formylhydrazine (3.06 g, 51,012 mmol, 1.5 equiv), HOBT (6.89 g, 51.012 mmol, 1.5 equiv), EDCI (9.78 g, 51.012 mmol, 1.5 equiv) and Et₃N (10.32 g, 102.024 mmol, 3 equiv) at room temperature. The resulting mixture was stirred for 1h at room temperature under nitrogen atmosphere. The reaction was quenched with sat. NH₄Cl (aq.) (100 mL) at room temperature. The aqueous layer was extracted with EtOAc (3×50 mL). The resulting mixture was concentrated under reduced pressure. The residue was purified by silica gel column chromatography elated with PE/EA (1:1) to afford 202b (7.5 g, 79.54%) as a white solid.

### Synthesis of 202c

Into a 100 mL 3-necked round-bottom flask were added 202b (1 g, 3.606 mmol, 1 equiv) and Lawesson Reagent (2.92 g, 7.212 mmol, 2 equiv) in THF (10 mL) at room temperature. The resulting mixture was stirred overnight at 40° C. under nitrogen atmosphere. The resulting mixture was concentrated under reduced pressure. The residue was purified by silica gel column chromatography eluted with PE/EA (5:1) to afford 202c (900 mg, 90.64%) as a colorless oil.

### Synthesis of 202d

Into a 50 ml, 3-necked round-bottom flask were added 202c (1 g, 3.632 mmol, 1 equiv) and Pd/C (0.2 g, 1.879 mmol, 0.52 equiv) in MeOH (10 mL) at room temperature. The resulting mixture was stirred overnight at room temperature under hydrogen atmosphere. The resulting mixture was filtered through filter paper and the filter cake was washed with MeOH (3×10 mL). The filtrate was concentrated under reduced pressure to afford 202d (800 mg, 89.78%) as white solid.

### Synthesis of 202e

Into a 100 mL 3-necked round-bottom flask were added 202d (800 mg, 3.261 mmol, 1 equiv), DCE (8 mL), 1-2 (933.56 mg, 3.261 mmol, 1 equiv), STAB (1382.18 mg, 6.522 mmol, 2 equiv) and AcOH (195.82 mg, 3.261 mmol, 1 equiv) at room temperature. The resulting mixture was stirred overnight at room temperature. The reaction was quenched by the addition of sat N4Cl (aq.) (10 mL) at room temperature. The aqueous layer was extracted with CH₂Cl₂ (3×10 mL). The resulting mixture was concentrated under reduced pressure. The residue was purified by silica gel column chromatography eluted with PE/EA (1:1) to afford 202e (800 mg, 47.58%) as a white solid.

### Synthesis of 202f

Into a 8 mL sealed tube were added 202e (500 mg, 0.970 mmol, 1 equiv), pyridine (459.78 mg, 5.820 mmol, 6 equiv), and DCM (5 mL) at room temperature. To the above mixture was added triphosgene (115.09 mg, 0.388 mmol, 0.4 equiv). The resulting mixture was stirred for 1 h at room temperature under nitrogen atmosphere. The reaction was quenched with saturated NaHCO₃ (aq.) (15 mL) at room temperature. The aqueous layer was extracted with DCM (3×10 mL). The resulting mixture was concentrated under reduced pressure. The residue was purified by Prep-TLC (PE/EA 1:1) to afford 202f (250 mg, 47.60%) as a yellow solid.

### Synthesis of 202

The 202f (250 mg) was purified by Chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 25% B to 25% B in 18.5 min; Wave Length: 220/254 nm; RT1 (min): 11.29; RT2 (min): 14.80; The first peak was the product; Sample Solvent: EtOH:DCM=1:1-HPLC; Injection Volume: 1.15 ml). This result in Compound 202 (96.8 mg, 38.72%) as a white solid.

LC-MS: (ES, m/z): [M+H]⁺ 542

H-NMR: (400 MHz, dmso-d6, δ ppm): 0.80-0.88 (m, 4H), 1.45-1.48 (m, 1H), 1.60-1.63 (m, 4H), 1.67-1.89 (m, 6H), 2.04-2.09 (m, 1H), 2.78 (s, 2H), 3.22-3.33 (m, 3H), 4.71-4.74 (d, 1H), 7.02 (s, 1H), 7.33-7.37 (m, 2H), 7.46-7.50 (m, 1H), 7.68 (s, 1H), 7.75-7.78 (d, 1H), 7.86-7.87 (d, 1H), 9.51 (s, 1H).

Alternatively, Compound 202 may be also prepared in the manner outlined below:

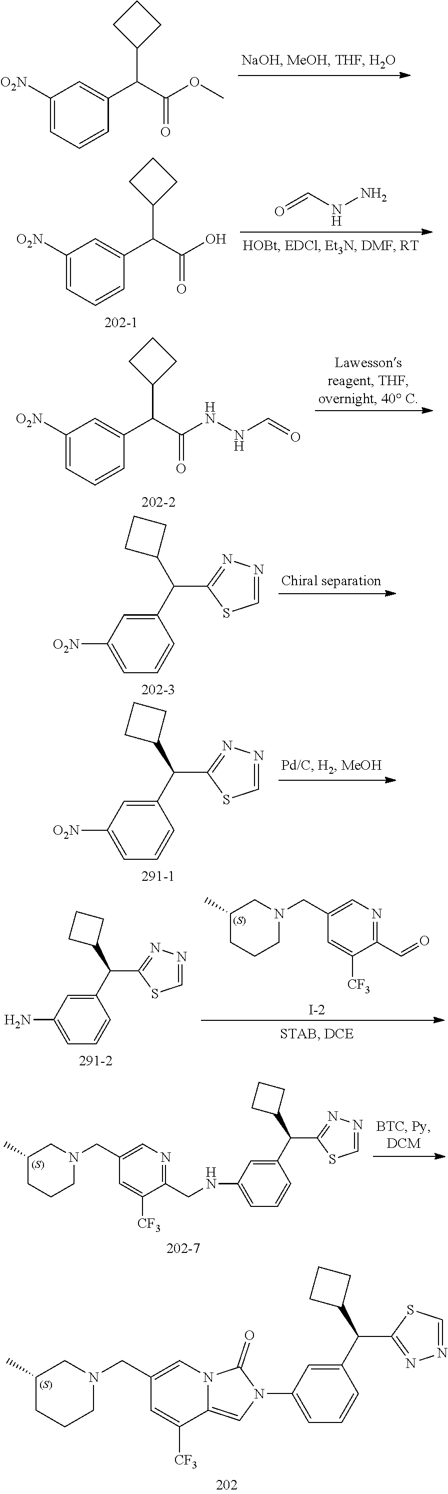

### 1. Synthesis of 202-1

To a stirred solution of methyl 2-cyclobutyl-2-(3-nitrophenyl)acetate (110 g, 441.296 mmol, 1 equiv) in MeOH (220 mL),THF (660 mL) and H₂O (220 mL) was added NaOH (52.95 g, 1323.888 mmol, 3 equiv) at room temperature. The resulting mixture was stirred for overnight at room temperature. The reaction was diluted with water (500 mL). The MeOH and THF was removed in vacuum. The resulting mixture was acidified to pH 2 with HCl (1M). The precipitated solids were collected by filtration and washed with water (3×200 mL). The solid was dried under vacuum. This resulted in 202-1 (102 g, 98.26%) as a white solid.

### 2, Synthesis of 202-2

To a stirred mixture of 202-1 (102 g, 433.602 mmol, 1 equiv), HOBT (117.18 g, 867.204 mmol, 2 equiv), EDCI (166.24 g, 867.204 mmol, 2 equiv) and Et₃N (131.63 g, 1300.806 mmol, 3 equiv) in DMF (1020 mL) were added N-formylhydrazine (78.12 g, 1300.806 mmol, 3 equiv) at room temperature. The resulting mixture was stirred overnight at room temperature. The reaction was quenched with sat. NH₄Cl (aq.)(31) at room temperature. The resulting mixture was extracted with EtOAc (2×1 L). The combined organic layers were concentrated under reduced pressure. The residue was purified by silica gel column chromatography, eluted with PE/EA (1:1) to afford crude product. The crude product (55 g) was purified by reverse flash chromatography with the following conditions: column, C18 silica gel; mobile phase, MeCN in Water (10 mmol/NH₄HCO₃), 20% to 80% gradient in 30 min; detector, UV 220 nm. This resulted in 202-2 (46 g, 38.26%) as a white solid,

### 3. Synthesis of 202-3

To a stirred solution of 202-2 (25.8 g, 93.047 mmol, 1 equiv) in TH-F (260 mL) was added Lawesson Reagent (75.27 g, 186.094 mmol, 2 equiv) at room temperature under nitrogen atmosphere. The resulting mixture was stirred for overnight at room temperature. The reaction was quenched by the addition of Water (260 mL) at room temperature. The resulting mixture was extracted with EtOAc (3×100 mL). The combined organic layers were was concentrated under reduced pressure. The residue was purified by silica gel column chromatography, eluted with PB/EA (5:1) to afford crude product. The crude product (18 g) was purified by reverse flash chromatography with the following conditions: column, C18 silica gel; mobile phase, MeCN in Water (10 mmol/L NH₄HCO₃), 20% to 65% gradient in 30 min; detector, UV 254 nm. This resulted in 202-3 (13.4 g, 52.31%) as a yellow oil.

### 4. Synthesis of 291-1

202-10 (10 g) was purified by Prep-Chiral-SFC with the following conditions (Column: (R, R)-WHELK-O1-Kromasil, 5*25 cm, 5 μm; Mobile Phase A: C02, Mobile Phase B: MeOH; Flow rate: 200 mL/min; Gradient: isocratic 35% B; Column Temperature (° C.): 35; Back Pressure (bar): 100; Wave Length: 220 nm; RT1 (min): 5.08; RT2 (min): 6.08; the second peak was product) to afford 291-1 (4.09 g, 40.90%) as a yellow oil.

### 5. Synthesis of 291-2

To a solution of 291-1 (4.09 g, 14.855 mmol, 1 equiv) in MeOH (120 mL) was added Pd/C (10%, 1.2g) under nitrogen atmosphere in a 500 ml round-bottom flask. The mixture was hydrogenated at room temperature for overnight under hydrogen atmosphere by using a hydrogen balloon, filtered through a Celite pad and concentrated under reduced pressure to afford 291-2 (3.65 g, 96.14%) as a brown solid.

### 6. Synthesis of 202-7

To a stirred solution of 291-2 (2.4 g, 9.782 mmol, 1 equiv) and I-2 (3.08 g, 10.760 mmol, 1.1 equiv) in DCE (70 mL) was added STAB (4.15 g, 19.564 mmol, 2 equiv). The resulting mixture was stirred for overnight at room temperature. The reaction was quenched by the addition of sat. NaHCO₃ (aq.) (100 mL). The resulting mixture was extracted with CH₂Cl₂ (2×100 mL). The combined organic layers were concentrated under reduced pressure. The residue was purified by reverse flash chromatography with the following conditions: column, C18 silica gel; mobile phase, MeCN in water (10 mmoL/L NH₄HCO₃ 40% to 90% gradient in 35 min; detector, L 254 nm. This resulted in 202-7 (4.3 g, 85.25%) as a white solid.

### 7. Synthesis of 202-0

To a stirred solution of 202-7 (4.3 g, 8.339 mmol, 1 equiv) and Pyridine (3.96 g, 50.034 mmol, 6 equiv) in DCM (120 mL) was added Triphosgene (0.99 g, 3.336 mmol, 0.4 equiv) at 0° C. under nitrogen atmosphere. The resulting mixture was stirred for 2 h at 0° C. under nitrogen atmosphere. The reaction was quenched by the addition of sat. NaHCO₃ (aq.) (100 mL). The resulting mixture was extracted with CH₂Cl₂ (3×100 mL). The combined organic layers were concentrated under reduced pressure. The residue was purified by reverse flash chromatography with the following conditions: column, C18 silica gel; mobile phase, MeCN in water (10 mmoL/L NH₄HCO₃), 40% to 90% gradient in 40 min; detector, UV 254 nm. This resulted in 202 (2.2 g, 48.71%) as a yellow solid.

LC-MS-202: (ES, m/z): [M+H]⁺ 542. H-NMR-202: (400 MHz, dmso-d6, δ ppm): 0.80-0.88 (m, 4H), 1.45-1.47 (m, 1H), 1.50-1.63 (m, 4H), 1.66-1.91 (m, 6H), 2.06-2.08 (m, 1H), 2.72-2.77 (m, 2H), 3.22-3.30 (m, 3H), 4.71-4.74 (d, 1H), 7.00 (s, 1H), 7.33-7.37 (m, 2H), 7.46-7.50 (m, 1H), 7.65 (s, 1H), 7.75-7.77 (d, 1H), 7.87 (s, 1H), 9.51 (s, 1H).

### Example 203. Synthesis of Compound 203

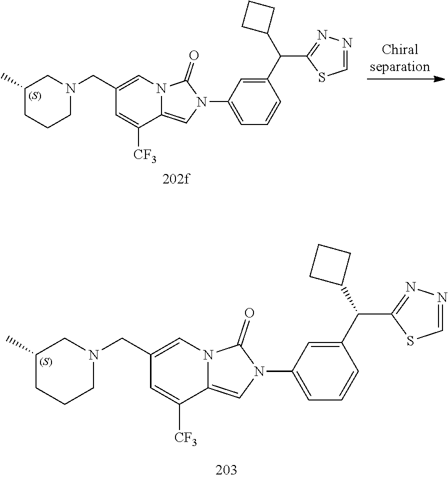

### Synthesis of 203

The 202f (250 mg) was purified by Chiral separation with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 25% B to 25% B in 18.5 min; Wave Length: 220/254 nm; RT1 (min): 11.29; RT2 (min): 14.80; The second peak was the product; Sample Solvent: EtOH:DCM=1:1-HPLC; Injection Volume: 1.15 mL; Number Of Runs: 4) to afford Compound 203 (97 mg, 38.80%) as a white solid,

LC-MS: (ES, m/z): [M+H]⁺ 542

H-NMR: (400 MHz, dmso-d6, δ ppm): 0.80-0.88 (m, 4H), 1.45-1.48 (m, 1H), 1.60-1.63 (m, 4H), 1.67-1.89 (m, 6H), 2.04-2.09 (m, 1H), 2.78 (s, 2H), 3.22-3.33 (m, 3H), 4.71-4.74 (d, 1H), 7.02 (s, 1H), 7.33-7.37 (m, 2H), 7.46-7.50 (m, 1H), 7.68 (s, 1H), 7.75-7.78 (d, 1H), 7.86-7.87 (d, 1H), 9.51 (s, 1H).

### Example 204. Synthesis of Compound 204

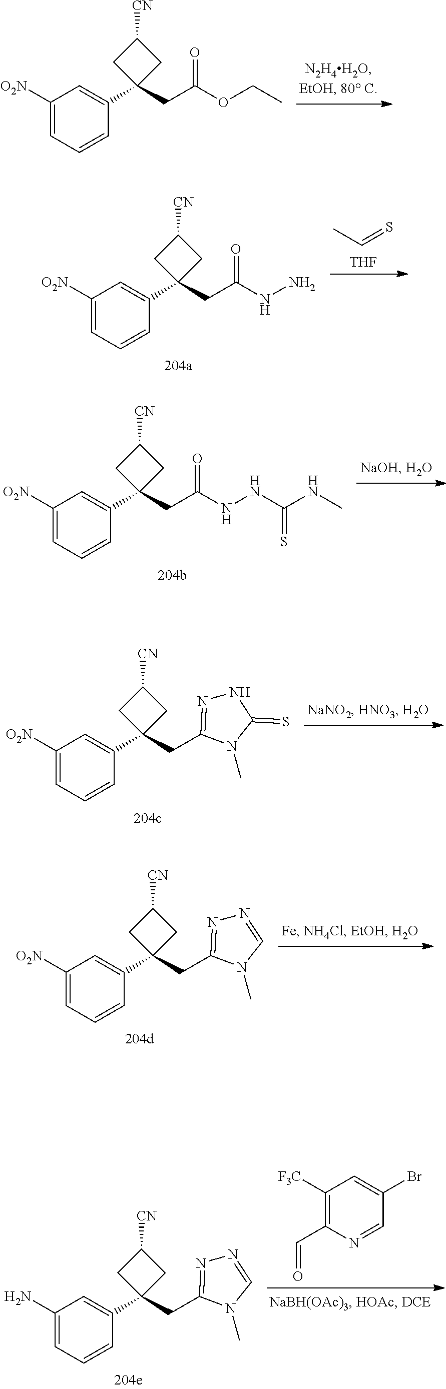

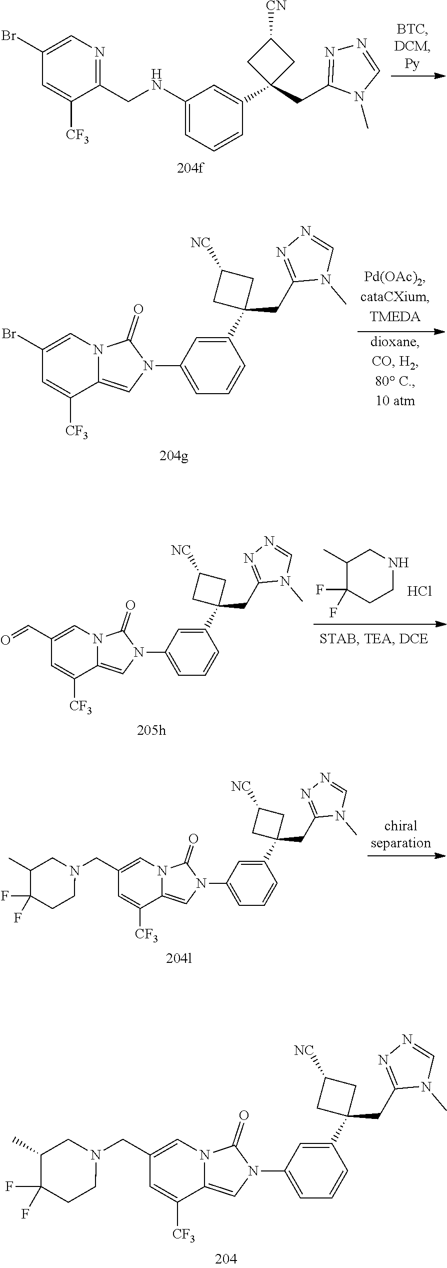

### 1. Synthesis of 204a

To a stirred solution of 204-1 (1.2 g, 4.162 mmol, 1 equiv) in EtOH (10 mL) was added hydrazine hydrate (98%) (1.04 g, 20.810 mmol, 5 equiv). The resulting mixture was stirred for overnight at 80° C. The resulting mixture was diluted with water (40 mL).The resulting mixture was extracted with CH₂Cl₂/MeOH=10/1 (3×40 mL), dried over anhydrous Na₂SO₄. After filtration, the filtrate was concentrated under reduced pressure. The combined organic layers were concentrated under reduced pressure. This resulted in 204a (1 g, 87.59%) as a white solid.

### 2. Synthesis of 204b

To a stirred solution of 204a (1 equiv) in tetrahydrofuran (20 mL) was added methyl isothiocyanate (2 equiv).The resulting mixture was stirred for overnight at room temperature. The resulting mixture was diluted with water (20 mL).The THF was removed under reduced pressure. The precipitated solids were collected by filtration and washed with water (2×5 mL),The resulting solid was dried under vacuum. This resulted in 204b (1.2 g, NaN) as a yellow solid.

### 3. Synthesis of 204c

Into a 100 mL round-bottom flask were added 204b (1.2 g, 3.454 mmol, 1 equiv) and NaOH (17.27 mL, 17.270 mmol, 5 equiv, 1M) at room temperature. The resulting mixture was stirred for overnight at room temperature. The mixture was acidified to pH 4 with HCl (1M).The precipitated solids were collected by filtration and washed with water (2 mL). The resulting solid was dried under vacuum. This resulted in 204c (860 mg, 75.59%) as a yellow solid.

### 4. Synthesis of 204d

To a stirred mixture of 204c (860 mg, 2.611 mmol, 1 equiv) and NaNO₂ (1801.44 ng, 26.110 mmol, 10 equiv) in H₂O (10 mL) and EtOAc (10 ml) was added HNO₃ (26.11 mL, 26.110 mmol, 10 equiv, 1M) dropwise at 0° C. The resulting mixture was stirred overnight at room temperature. The mixture basified to pH 8 with saturated NaHCO₃ (aq.). The resulting mixture was extracted with CH₂Cl₂/MeOH=10/1 (3×40 mL). The combined organic layers were dried over anhydrous Na₂SO₄. After filtration, the filtrate was concentrated under reduced pressure. This resulted in 204d (710 mg, 91.46%) as a yellow solid.

### 5. Synthesis of 204e

To a stirred mixture of 204d (710 mg 2.388 mmol, 1 equiv) in EtOH (20 mL) and H₂O (5 mL) were added NH₄Cl (510.95 mg, 9.552 mmol, 4 equiv) and Fe (400.08 mg, 7.164 mmol, 3 equiv). The resulting mixture was stirred for 3 h at 80° C. The resulting mixture was filtered, the filter cake was washed with CH₂Cl₂ (2×10 mL). The resulting mixture was diluted with water (80 mL). The resulting mixture was extracted with CH₂Cl₂ (3×100 mL). The combined organic layers were dried over anhydrous Na₂SO₄. After filtration, the filtrate was concentrated under reduced pressure. This resulted in 204e (700 mg, 109.65%) as a yellow solid.

### 6. Synthesis of 204f

To a stirred solution of 204e (700 ng, 2,618 mmol, 1 equiv) and 5-bromo-3-(trifluoromethyl)picolinaldehyde (798.12 mg, 3.142 mmol, 1.2 equiv) in DCE (15 mL) were added HOAc (157.24 mg, 2,618 mmol, 1 equiv) and NaBH(OAc)3 (832.43 mg 3.927 mmol, 1.5 equiv).The resulting mixture was stirred for overnight at room temperature. The reaction was quenched by the addition of Water (15 mL). The resulting mixture was extracted with CH₂Cl₂/MeOH=10/1 (3×20 mL). The combined organic layers were dried over anhydrous Na₂SO₄. After filtration, the filtrate was concentrated under reduced pressure. The residue was purified by trituration with methyl tert-butyl ether (5 mL). This resulted in 204f (900 ng, 68.02%) as a light yellow solid.

### 7. Synthesis of 204g

To a stirred solution of 204f (900 mg, 1,781 mmol, 1 equiv) and Pyridine (845.25 mg, 10,686 mmol, 6 equiv) in DCM (25 mL) was added Triphosgene (211.40 ng, 0.712 mmol, 0.4 equiv) at 0° C. The resulting mixture was stirred for 2 h at 0° C. The reaction was quenched by the addition of NaHCO₃ (aq.) (20 mL). The resulting mixture was extracted with CH₂Cl₂/MeOH (3×20 mL). The combined organic layers were concentrated under reduced pressure. The residue was purified by trituration with methyl tert-butyl ether (5 mL),This resulted in 204g (700 mg, 73.97%) as a yellow solid.

### 8. Synthesis of 204h

To a solution of 204g (700 mg, 1.317 mmol, 1 equiv), TMEDA (306.18 mg, 2.634 mmol, 2 equiv) in 1,4-dioxane (20 mL) was added and Pd(OAc)2 (29.58 mg, 0.132 mmol, 0.1 equiv) in an autoclave. After flushing the autoclave three times with CO/H₂ (1:1), the mixture was pressurized to 10 atm with CO/H₂ (1:1) at 80 degrees C. for overnight. The resulting mixture was concentrated under vacuum. The residue was purified by silica gel column chromatography, eluted with CH₂Cl₂/MeOH (20:1) to CH₂Cl₂/MeOH (5:1) to afford 204h (489 mg, 77.26%) as a yellow solid.

### 9. Synthesis of 204i

To a stirred mixture of 204h (300 mg, 0,624 mmol, 1 equiv) and 4,4-difluoro-3-methylpiperidine hydrochloride (321.49 mg, 1.872 mmol, 3 equiv) in DCE (10 mL) were added TEA (252.74 mg, 2.496 mmol, 4 equiv) and STAB (264.68 mg, 1.248 mmol, 2 equiv).The resulting mixture was stirred for overnight at room temperature. The reaction was quenched by the addition of Water (10 mL) at room temperature. The resulting mixture was extracted with CH₂Cl₂ (3×15 mL). The combined organic layers were concentrated under reduced pressure. The residue was purified by Prep-TLC (CH₂Cl₂/MeOH 15:1) to afford 204i (150 mg, 40.06%) as a yellow solid.

### 10. Synthesis of 204

204 (150 mg) was purified by Prep-Chiral-HPLC with the following conditions (Column: CHIRALPAK IC, 2*25 cm, 5 μm; Mobile Phase A: Hex (0.5% 2M N43-MeOH), Mobile Phase B: MeOH:DCM=1:1 (0.1% 2M NH₃-MeOH); Flow rate: 20 mL/min; Gradient: 50% B to 50% B in 13 min; Wave Length: 220/254 nm; RT1 (min): 7.48; the first peak is product) to afford 204 (49.9 mg, 33.27%) as a yellow solid.

LC-MS-204: (ES, m/z): [M+H]⁺ 600. H-NMR: (400 MHz, DMSO-d6, ppm): δ 0.82-0.86 (d, 3H), δ 1.85-2.19 (m, 4H), δ 2.26-2.33 (m, 1H), δ 2.64-2.67 (m, 3H), δ 2.69-2.79 (m, 4H), δ 3.00-3.06 (m, 2H), δ 3.26 (s, 2H), δ 3.35 (s, 2H), δ 3.74-3.78 (t, 1H), δ 6.73-6.75 (d, 1H), δ 7.03 (s, 1H), δ 7.25 (s, 1H), δ 7.32-7.39 (m, 2H), δ 7.70-7.73 (m, 1H), δ 8.16 (s, 1H).

### Example 205. Synthesis of Compound 205

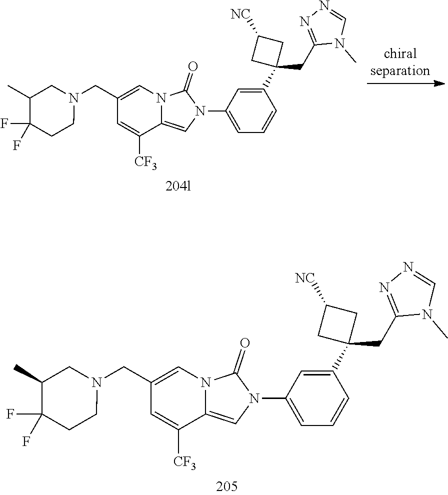 [SMILES: CC1CN(Cc2cc(C(F)(F)F)c3cn(-c4cccc([C@]5(Cc6nncn6C)C[C@H](C#N)C5)c4)c(=O)n3c2)CCC1(F)F ; C[C@H]1CN(Cc2cc(C(F)(F)F)c3cn(-c4cccc([C@]5(Cc6nncn6C)C[C@H](C#N)C5)c4)c(=O)n3c2)CCC1(F)F]

### 1. Synthesis of 205

205 (150 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: MeOH:DCM=1:1 (0.1% 2M NH3-MEOH); Flow rate: 20 mL/min; Gradient: 50% B to 50% B in 13 min; Wave Length: 220/254 nm; RT2 (min): 9.73; the second peak is product) to afford 205 (52.7 mg, 35.13%) as a yellow solid.

LC-MS: (ES, m/z): [M+H]⁺ 600

H-NMR: (400 MHz, DMSO-d6, ppm): δ 0.86-0.89 (d, 3H), δ 1.89-2.11 (m, 4H), δ 2.26-2.33 (m, 1H), δ 2.64-2.67 (m, 2H), δ 2.69-2.79 (m, 5H), δ 3.00-3.06 (m, 2H), δ 3.26 (s, 2H), δ 3.35 (s, 2H), δ 3.74-3.78 (t, 1H), δ 6.73-6.75 (d, 1H), δ 7.03 (s, 1H), δ 7.25 (s, 1H), δ 7.32-7.39 (m, 2H), δ 7.70-7.73 (m, 1H), δ 8.16 (s, 1H).

### Example 206. Synthesis of Compound 206

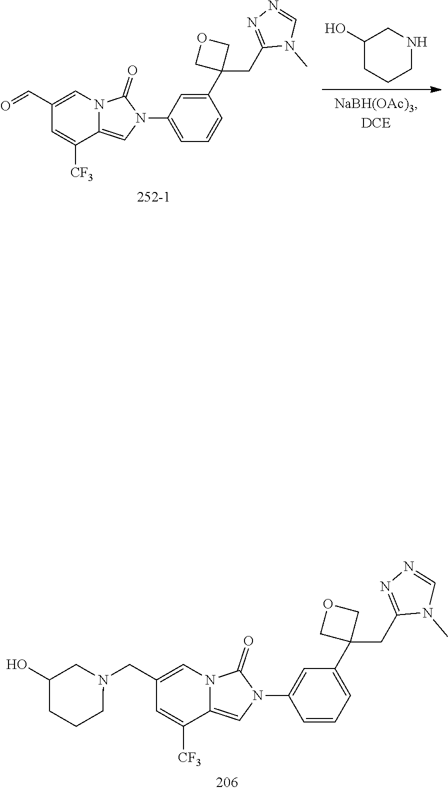 [SMILES: Cn1cnnc1CC1(c2cccc(-n3cc4c(C(F)(F)F)cc(C=O)cn4c3=O)c2)COC1 ; OC1CCCNC1 ; Cn1cnnc1CC1(c2cccc(-n3cc4c(C(F)(F)F)cc(CN5CCCC(O)C5)cn4c3=O)c2)COC1]

To a stirred solution of 252-1 (90 ng, 0.19 mmol, 1.0 equiv) and piperidin-3-ol (39.8 mg, 0.39 mmol, 2.0 equiv) in DCE was added NaBH(OAc)3 (83.4 mg, 0.39 mmol, 2.0 equiv) at room temperature. The resulting mixture was stirred for overnight at rom temperature. The reaction was quenched with water (20 mL) at room temperature. The resulting mixture was extracted with CH2Cl2 (3×20 mL). The combined organic layers were concentrated under reduced pressure. The residue was purified by Prep-TLC (CH2Cl2/MeOH=10:1) to afford 206 (51 mg, 44%) as a yellow solid.

LC-MS: (ES, m/z): [M+H]⁺ 543. H-NMR: (400 MHz, DMSO, δ ppm): 1.06-1.11 (m, 1H), 139-1.45 (m, 1H), 1.61-1.64 (m, 1H), 1.73-1.80 (m, 2H), 1.88-1.93 (m, 1H), 2.65-2.67 (m, 1H), 2.80-2.86 (m, 1H), 2.91 (s, 3H), 3.23-3.27 (m, 1H), 3.31-3.33 (m, 1H), 3.46-3.53 (m, 1H), 3.61 (s, 2H), 4.62 (s, 1H), 4.91-4.96 (m, 4H), 6.88-6.90 (d, 1H), 7.01 (s, 1H), 7.32 (s, 1H), 7.38-7.42 (m, 2H), 7.67 (s, 1H), 7.74-7.76 (m, 1H), 8.18-8.22 (m, 2H).

### Example 207. Synthesis of Compound 207

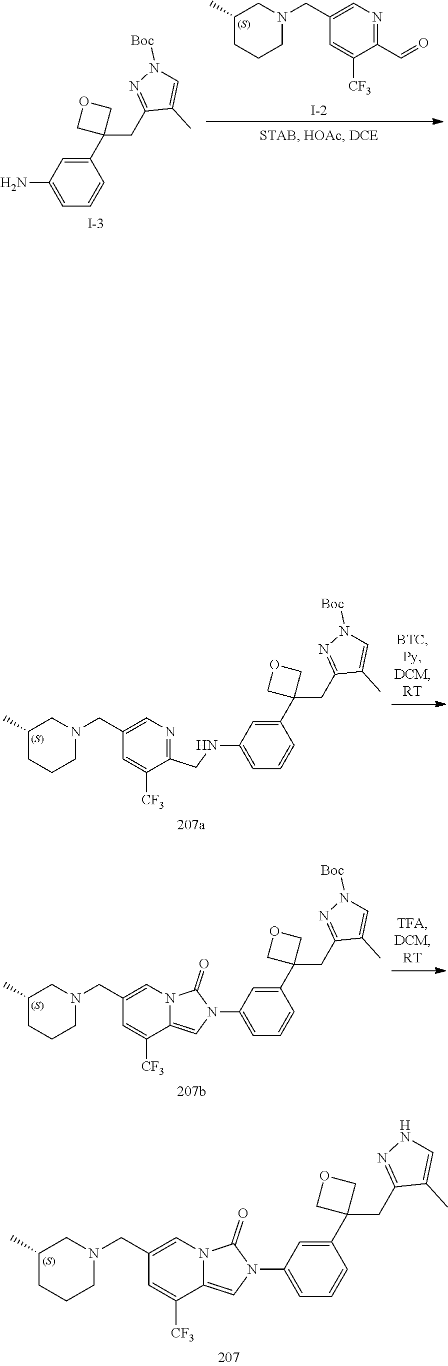

### 1. Synthesis of 207a

To a stirred solution of I-3 (200 mg, 0.582 mmol, 1.00 equiv) and I-2 (166.73 Mg 0.582 mmol, 1 equiv) in DCE (5 mL) was added HOAc (34.97 mg, 0.582 mmol, 1 equiv) and STAB (246.85 mg, 1.164 mmol, 2 equiv) at room temperature. The resulting mixture was stirred for 3h at room temperature. The reaction was quenched by the addition of saturated NaHCO₃ (aq.) (50 mL) at room temperature. The aqueous layer was extracted with DCM (2×20 mL). The organic layers were concentrated under reduced pressure. The residue was purified by Prep-TLC (DCM/MeOH 20:1) to afford 207a (200 mg, 52.04%) as a light yellow oil.

### 2. Synthesis of 207b

To a stirred solution of 207a (180 mg, 0.293 mmol, 100 equiv) and Pyridine (463.98 mg 5%860 mmol, 20 equiv) in DCM (10 mL) was added Triphosgene (43.52 mg 0.146 mmol, 0.5 equiv) at room temperature. The resulting mixture was stirred for 10 min at room temperature. The reaction was quenched with saturated NaHCO₃ (aq.) (50 mL) at room temperature. The aqueous layer was extracted with DCM (2×:20 mL). The organic layers were concentrated under reduced pressure. The residue was purified by Prep-TLC (DCM/MeOH 15:1) to afford 207b (150 mg, 74.35%) as a yellow solid,

### 3. Synthesis of 207

To a stirred solution of 207b (150 mg, 0.234 mmol, 1.00 equiv) in DCM (5 mL) was added TFA (1 mL) at room temperature. The resulting mixture was stirred for 30 min at room temperature. The resulting mixture was concentrated under vacuum. The crude product was purified by Prep-HPLC with the following conditions (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 54% B to 73% B in 8 mi, Wave Length: 220 nm; RT1 (min): 7.85) to afford 207 (35.6 mg, 27.29%) as a yellow solid.

LC-MS: (ES, m/z): [M+H]⁺ 540. H-NMR: (400 MHz, CD₃OD, δ ppm): 0.90-0.98 (m, 4H), 1.29-1.42 (m, 3H), 1.56-1.77 (m, 5H), 1.96-2.01 (m, 1H), 2.83-2.91 (m, 2H), 3.34-3.50 (m, 4H), 5.02 (s, 4H), 6.90-6.92 (m, 2H), 7.10-7.12 (m, 2H), 7.21 (s, 1H), 7.42-7.46 (m, 1H), 7.62-7.64 (d, 1H), 7.67 (s, 1H).

### Example 208. Synthesis of Compound 208

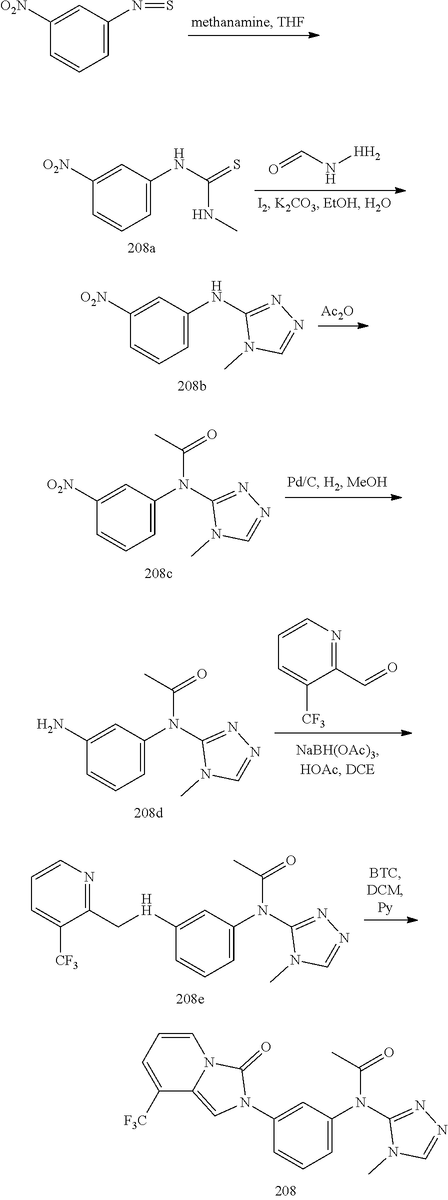

### Synthesis of 208a

To a stirred solution of 1-isothiocyanato-3-nitrobenzene (5 g, 27,750 mmol, 1 equiv) and THE (50 mL) was added methamine (13.87 mL, 27.750 mmol, 1 equiv, 2M in THF) at room temperature under nitrogen atmosphere. The resulting mixture was stirred for 2h at room temperature under nitrogen atmosphere. The reaction was quenched with Water (100 mL) at room temperature. The precipitated solids were collected by filtration and washed with water (2×20 mL) to afford 208a (4 g, 68.24%) as a white solid.

### Synthesis of 208b

To a stirred solution of 208a (4 g, 18,936 mmol, 1 equiv) and N-formylhydrazine (2.84 g, 47.340 mmol, 2.5 equiv) in EtOH (62 mL) and 120 (15 mL) were added K₂CO₃ (6.54 g, 47.340 mmol, 2.5 equiv) and I₂ (5.77 g, 22.723 mmol, 1.2 equiv) at room temperature under nitrogen atmosphere. The resulting mixture was stirred for 1h at room temperature. The reaction was quenched with Water (200 mL) at room temperature. The aqueous layer was extracted with EtOAc (5×40 mL). The resulting mixture was concentrated under reduced pressure. The residue was purified by silica gel column chromatography, eluted with CH₂Cl₂/MeOH (20:1) to afford 208b (860 mg, 20.72%) as a yellow solid.

### Synthesis of 208e

A solution of 208b (800 mg, 3.650 mmol, 1 equiv) in acetic anhydride (5 mL) was stirred for overnight at room temperature under nitrogen atmosphere. The reaction was quenched with NaHCO₃ (aq.) (100 mL) at 0° C. The aqueous layer was extracted with EtOAc (3×40 mL). The resulting mixture was concentrated under reduced pressure. The residue was purified by Prep-TLC (CH₂Cl₂/MeOH 20:1) to afford 208c (300 mg, 31.47%) as yellow oil.

### Synthesis of 208d

To a solution of 208c (200 mg, 0.766 mmol, 1 equiv) in MeOH (4 mL) was added Pd/C (10%, 40 mg) under nitrogen atmosphere. The mixture was hydrogenated at room temperature for overnight under hydrogen atmosphere using a hydrogen balloon, filtered through a Celite pad and concentrated under reduced pressure. This resulted in 208d (140 mg, 79.08%) as white solid.

### Synthesis of 208e

A solution of 208d (130 mug, 0.562 mmol, 1 equiv) in DCE (2 mL) was treated with 3-(trifluoromethyl)pyridine-2-carbaldehyde (147.65 mg, 0.843 mmol, 1.5 equiv) for 30 min at room temperature under nitrogen atmosphere followed by the addition of NaBH(OAc)3 (238.28 mg, 1.124 mmol, 2 equiv) and HOAc (33.76 mg, 0,562 mmol, 1 equiv) at room temperature. The reaction was quenched by the addition of NH₄Cl (aq.) (20 mL) at room temperature. The aqueous layer was extracted with EtOAc (3×7 mL). The resulting mixture was concentrated under reduced pressure. The residue was purified by Prep-TLC (CH₂Cl₂/MeOH 20:1) to afford 208e (140 mg, 63.80%) as yellow solid,

### Synthesis of 208

To a stirred solution of 208e (140 mg, 0.359 mmol, 1 equiv) and Pyridine (170.21 ng, 2.154 mmol, 6 equiv) in DCM (6 mL) was added triphosgene (37.25 mg, 0,126 mmol, 0.35 equiv) at 0° C. The resulting mixture was stirred for 30 min at room temperature under nitrogen atmosphere. The reaction was quenched with NH₄Cl (aq.) (10 mL) at room temperature. The aqueous layer was extracted with DCM (3×:20 mL). The resulting mixture was concentrated under reduced pressure. The residue was purified by Prep-TLC (CH₂Cl₂/MeOH 10:1) to afford 208 (80 mg, crude). The crude product (80 mg) was purified by Prep-HPLC with the following conditions (Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 21% B to 40% B in 8 min, Wave Length: 220 nm; RT1 (min): 7.40) to afford 208 (41.4 mg, 27.73%) as a yellow solid.

LC-MS: (ES, m/z): [M+H]⁺ 417. H-NMR: (400 MHz, DMSO, δ ppm): 2.07 (s, 3H), 3.56 (s, 3H), 6.27-6.31 (t, 1H), 7.10-7.12 (d, 1H), 7.42 (s, 2H), 7.57-7.61 (m, 1H), 7.79-7.81 (d, 1H), 7.83-88.12 (m, 2H), 8.56 (s, 1H).

### Example 209. Synthesis of Compound 209

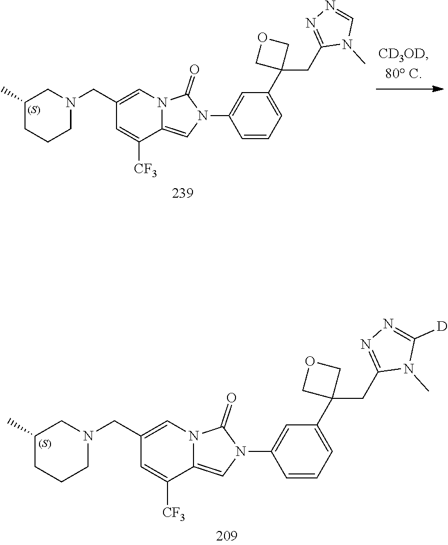

### Synthesis of 209

A solution of 239 (180 ng, 0,333 mmol, 1 equiv) in CD₃OD (3 mL) was stirred for 2 h at 80° C. The resulting mixture was concentrated under reduced pressure. The residue was purified by Prep-TLC (CH₂Cl₂/MeOH=15:1) to afford 209 (97.9 mg, 53.74%) as a yellow solid.

LCMS: (ES, m/z): [M+H]⁺ 542. H-NMR: (400 MHz, DMSO-d₆, ppm, δ): 0.84-0.97 (m, 4H), 1.36-1.59 (m, 1H), 1.59-1.83 (m, 4H), 1.80-1.90 (m, 1H), 2.75-2.86 (m, 2H), 3.01 (s, 3H), 3.25-3.33 (m, 2H), 3.53 (s, 2H), 4.87-4.96 (m, 4H), 6.88-6.90 (m, 1H), 7.02 (s, 1H), 7.32 (s, 1H), 7.38-7.41 (m, 2H), 7.66 (s, 1H), 7.74-7.76 (m, 2H).

### Example 210. Synthesis of Compound 210

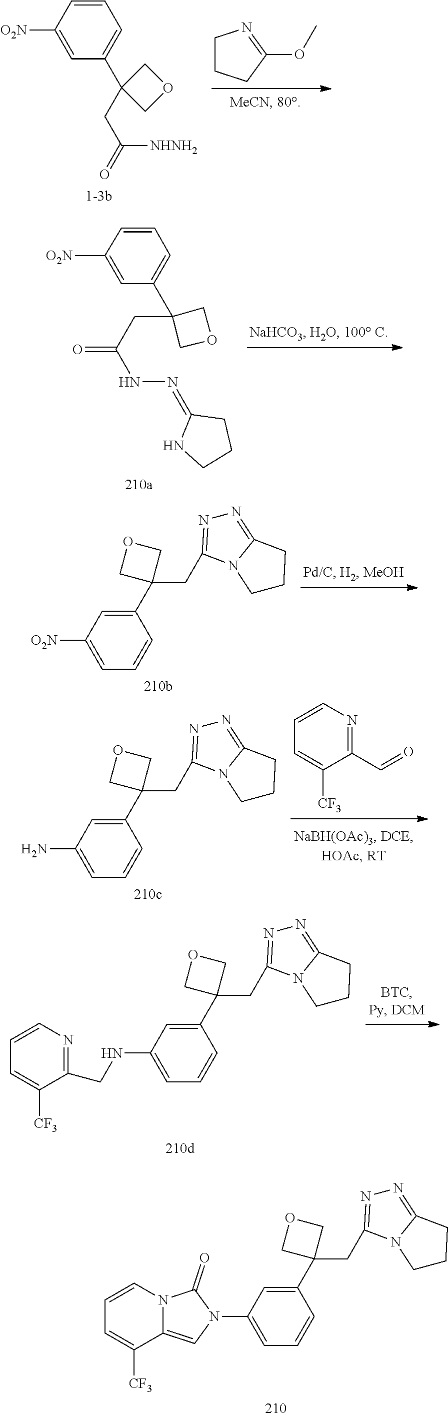

### 1. Synthesis of 210

A solution of 1-3b (1.8 g, 7.164 mmol, 1 equiv) in MeCN (30 mL) was stirred 2-methoxy-4,5-dihydro-3H-pyrrole (1.07 g, 10.746 mmol, 1.5 equiv) at room temperature under nitrogen atmosphere. The resulting mixture was stirred overnight at 80° C. under nitrogen atmosphere. The resulting mixture was concentrated under reduced pressure. This resulted in 210a (1.8 g, 63.14%) as a yellow solid. The crude product was used in the next step directly without further purification.

### 2. Synthesis of 210

To a stirred solution of 210a (1.8 g, 5.654 mmol, 1 equiv) in H2O (30 mL) was added NaHCO₃ (4.75 g, 56.540 mmol, 10 equiv) at room temperature. The resulting mixture was stirred overnight at 100° C. The resulting mixture was diluted with water (50 mL). The aqueous layer was extracted with EtOAc (5×30 mL). The organic layers were concentrated under reduced pressure. The residue was purified by Prep-TLC (DCM/MeOH 12:1) to afford 210b (200 mg, 9.42%) as a yellow solid.

### 3. Synthesis of 210c

To a solution of 210b (180 mg, 0.599 mmol, 1 equiv) in 10 mL MeOH was added Pd/C (20 mg, 10%) under nitrogen atmosphere in a 50 mL round-bottom flask. The mixture was hydrogenated at room temperature for 2h under hydrogen atmosphere using a hydrogen balloon, filtered through a Celite pad and concentrated under reduced pressure. This resulted in 210c (100 mg, 49.37%) as an off-white solid. The crude product was used in the next step directly without further purification.

### 4. Synthesis of 210d

To a stirred solution of 210c (80 mg, 0.296 mmol, 1 equiv) and 3-(trifluoromethyl)pyridine-2-carbaldehyde (51.82 mg, 0.296 mmol, 1 equiv) in DCE (3 mL) was added NaBH(OAc)3 (125.44 mg, 0.592 mmol, 2 equiv) and HOAc (17.77 mg, 0,296 mmol, 1 equiv) at room temperature under nitrogen atmosphere. The resulting mixture was stirred for 3h at room temperature. The resulting mixture was concentrated under reduced pressure. The residue was purified by Prep-TLC (DCM/MeOH 15:1) to afford 210d (50 mg, 35.80%) as an off-white solid.

### 5. Synthesis of 210

To a stirred solution of 210d (40 mg, 0.093 mmol, 1 equiv) and Pyridine (73.68 mg, 0.931 mmol, 10.00 equiv) in DCM (5 mL) were added Triphosgene (9.67 mg, 0.033 mmol, 0.35 equiv) at room temperature. The reaction was quenched with saturated NaHCO₃ (aq.) (30 mL) at room temperature. The aqueous layer was extracted with DCM (2×10 ml). The organic layers were concentrated under reduced pressure. The residue was purified by reverse flash chromatography with the following conditions: column, C18 silica gel; mobile phase, MeCN in water, 0% to 50% gradient in 20 min; detector, UV 254 nm. This resulted in 210 (19.2 mg, 43.00%) as a yellow solid.

LC-MS: (ES, m/z): [M+H]⁺ 456. H-NMR: (400 MHz, CD₃OD, δ ppm): 2.37-2.45 (m, 2H), 2.75-2.78 (m, 2H), 2.94-2.97 (m, 2H), 3.59 (s, 2H), 5.00-5.03 (m, 2H), 5.03-5.07 (m, 2H), 6.33-6.37 (m, 1H), 6.93-6.96 (d, 1H), 7.04-7.05 (d, 1H), 7.11 (s, 1H), 7.24 (s, 1H), 7.50-7.54 (m, 1H), 7.64-7.66 (d, 1H), 7.75-7.77 (d, 1H).

### Example 211. Synthesis of Compound 211

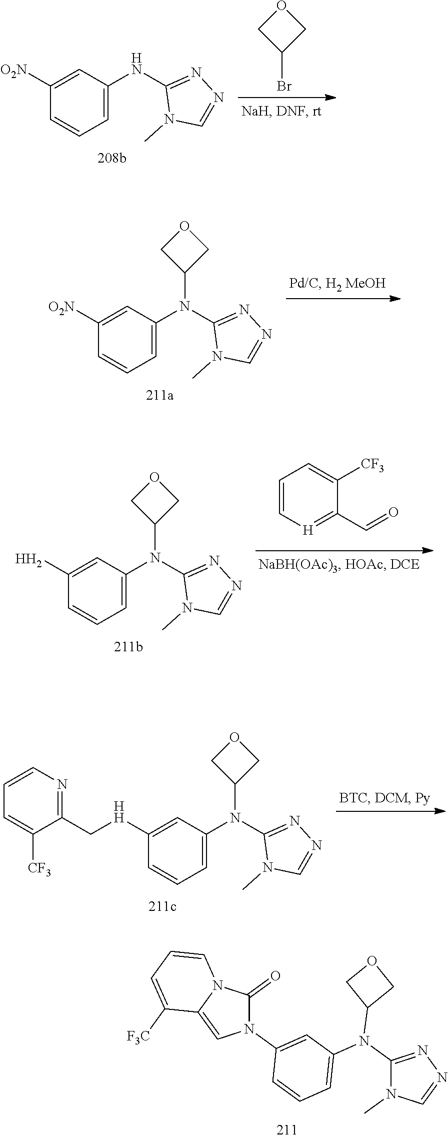

### 1. Synthesis of 211a

To a solution of 208b (800 mg, 3.650 mmol, 1 equiv) in DMF (10 mL) was added NaH (218.95 mg, 5.475 mmol, 1.5 equiv, 60% in oil) at 0 degrees C. The resulting mixture was stirred for 1h at 0 degrees C. To the above 3-bromooxetane (999.81 mg, 7.300 mmol, 2 equiv) was added. The resulting mixture was allowed to warm to RT and stirred for overnight. The reaction mixture was quenched by water (30 mL) and extracted with DCM (3*25 mL). The resulting mixture was concentrated under reduced pressure. The residue was purified by Prep-TLC (CH₂Cl₂/MeOH 9:1) to afford 21 Ia (134 mg, 13.34%) as yellow oil.

### 2, Synthesis of 211b

To a solution of 211a (120 mg, 0,436 mmol, 1 equiv) in MeOH (2 mL) was added Pd/C (10%, 30 mg) under nitrogen atmosphere in a 50 mL round-bottom flask The mixture was hydrogenated at room temperature for overnight under hydrogen atmosphere using a hydrogen balloon, filtered through a Celite pad and concentrated under reduced pressure. This resulted in 211b (103 mg, 96.32%) as yellow solid.

### 3. Synthesis of 211c

A solution of 211b (103 mg 0.420 mmol, 1 equiv) in DCE (2 mL) was treated with 3-(trifluoromethyl)pyridine-2-carbaldehyde (88.24 mg, 0.504 mmol, 1.2 equiv) for 30 min at room temperature followed by the addition of NaBH(OAc)₃ (178.00 mg, 0.840 mmol, 2 equiv) and HOAc (25.22 mg, 0.420 mmol, 1 equiv). The resulting mixture was stirred for overnight. The reaction was quenched by the addition of NH₄Cl (aq.) (20 mL) at room temperature. The aqueous layer was extracted with EtOAc (3×7 mL). The resulting mixture was concentrated under reduced pressure. The residue was purified by Prep-TLC (CH₂Cl₂/MeOH 20:1) to afford 211c (89.9 ng, 52.94%) as yellow solid,

### 4, Synthesis of 211

To a stirred solution of 211c (80 mg, 0.198 mmol, 1 equiv) and Pyridine (9189 mg, 1.188 mmol, 6 equiv) in DCM (2 mL) was added Triphosgene (20.55 mg, 0.069 mmol, 0.35 equiv) at 0° C. The resulting mixture was stirred for 30 min at room temperature. The reaction was quenched with sat. NaHCO3 (aq.)(10 mL) at room temperature. The resulting mixture was extracted with DCE/MeOH (10:1) (3×20 mL). The combined organic layers were concentrated under reduced pressure. The residue was purified by Prep-TLC (CH₂Cl₂/MeOH 10:1). The crude product (80 mg) was purified by Prep-HPLC with the following conditions (Column: XBridge Prep OBD C18 Column, 30* 150 mm, 5 μm; Mobile Phase A: Water (10 mmol/L NH₄HCO₃), Mobile Phase B: ACN; Flow rate: 60 mL/min; Gradient: 16% B to 46% B in 8 min, 46% B; Wave Length: 220 nm; RT11 (min): 7.65) to afford 211 (35.7 mg, 41.93%) as a yellow solid.

LC-MS: (ES, m/z): [M+H]⁺ 431. H-NMR: (400 MHz, DMSO, δ ppm): 3.42 (s, 3H), 4.42-4.45 (t, 2H), 4.77-4.80 (t, 2H), 5.18-5.20 (t, 1H), 6.26-6.29 (m, 1H), 6.49-6.52 (m, 1H), 7.08-7.10 (d, 1H), 7.17 (s, 1H), 7.33-7.42 (m, 3H), 7.78-7.79 (d, 1H), 8.57 (s, 1H).

### Example 212. Synthesis of Compound 212

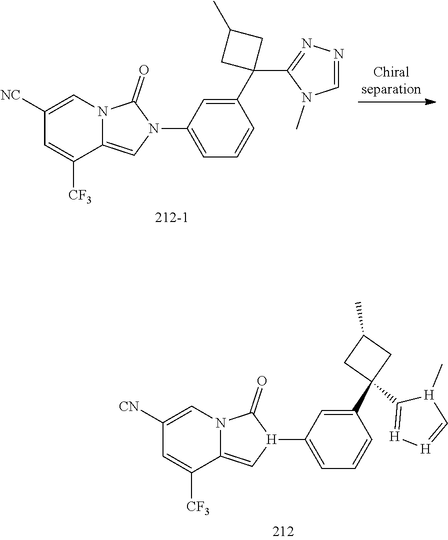

### Synthesis of 212

The 212-1 (240 mg) was purified by chiral separation with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 90% B to 90% B in 17 min; Wave Length: 220/254 nm; RT2 (min): 11.69; RT2 (min): 14.54, the first peak is product) to afford 212 (129.8 mg, 33.30%) as a yellow solid.

LC-MS: (ES, m/z): [M+H]⁺ 453. H-NMR: (400 MHz, DMSO-d6, ppm, δ): 1.08-1.09 (m, 3H), 2.55-2.58 (m, 3H), 2.87-2.89 (m, 2H), 3.23-3.25 (s, 3H), 7.27-7.31 (m, 2H), 7.50-7.52 (m, 1H), 7.54-7.58 (m, 1H), 7.63-7.65 (m, 1H), 7.80 (s, 1H), 8.41 (s, 1H) 8.70 (s, 1H).

### Example 213. Synthesis of Compound 213

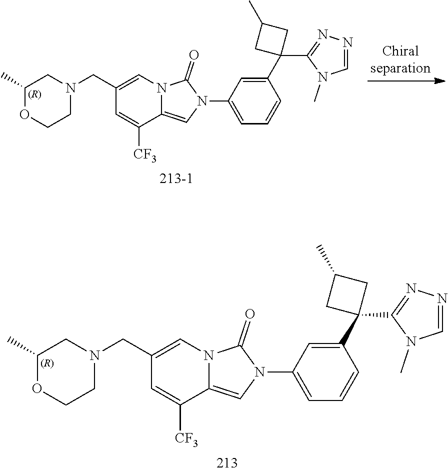

### Synthesis of 213

The 213-1 (350 mg) was purified by Chiral separation with the following conditions Column: CHIRALPAK IC, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 65% B to 65% B in 20 min; Wave Length: 220/254 nm; RT1 (min): 8.96; RT2 (min): 13.78; The second peak was the product. Sample Solvent: EtOH:DCM=1:1-HPLC; Injection Volume: 2 mL; Number of Runs: 6 to afford crude product. The crude product was purified by Prep-HPLC with the following conditions (Column: XBridge Prep OBD C18 Column, 30*150 mam, 5 μm; Mobile Phase A: Water (10 mmol/L. NH₄HCO₃), Mobile Phase B: ACN; Flow rate: 60 mL/min; Gradient: 29% B to 55% B in 8 min, 55% B; Wave Length: 220 nm; RT1 (min): 7.73) to afford 213 (71.9 mg, 21.50%) as a yellow solid.

### Example 214. Synthesis of Compound 214

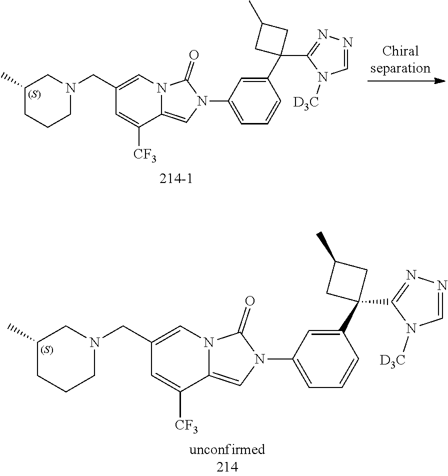

### Synthesis of 214

The 214-1 (400 mg) was purified by Prep-Chiral-HPLC with the following conditions (Column: CHIRALPAK AD-H, 2*25 cm, 5 μm; Mobile Phase A: Hex (0.5% 2M NH₃-MeOH), Mobile Phase B: EtOH; Flow rate: 20 mL/min; Gradient: 15% B to 15% B in 28 min; Wave Length: 220/254 nm; RT2 (min): 11.87, RT2 (min): 16.98, the second peak is product) to afford 214 (25.7 mg, 31.87%) as a yellow solid.

LC-MS: (ES, m/z): [M+H]⁺ 542. H-NMR: (400 MHz, DMSO-d₆, δ ppm): 0.81-0.93 (m, 4H), 1.09-1.11 (d, 3H), 1.43-1.46 (m, 1H), 1.57-1.66 (m, 4H), 1.86-1.91 (m, 1H), 2.29-2.46 (m, 3H), 2.72-2.77 (m, 2H), 3.09-3.14 (m, 2H), 3.24 (s, 2H), 7.00 (s, 1H), 7.07-7.09 (d, 1H), 7.36 (s, 1H), 7.44-7.48 (m, 1H), 7.60-7.65 (m, 2H), 7.71 (s, 1H), 8.36 (s, 1H).

### Example 215. Synthesis of Compound 215

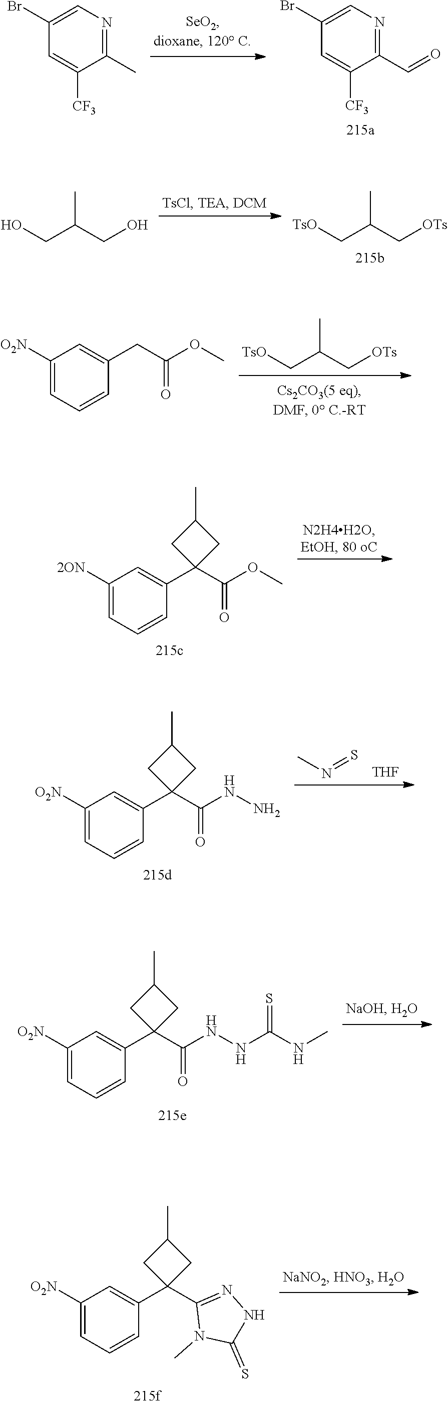

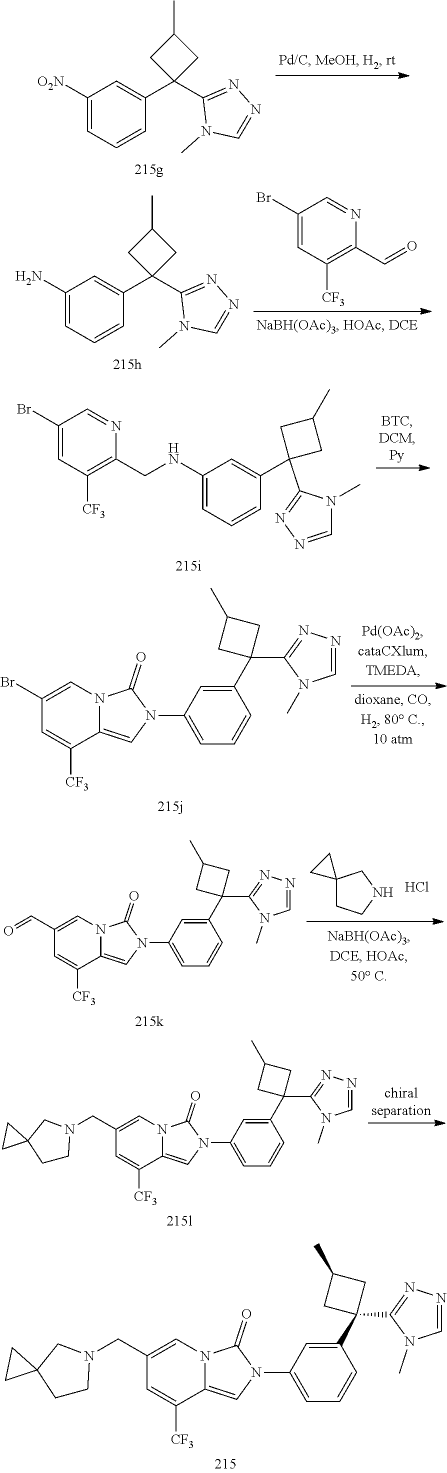

### 1. Synthesis of 215a

To a stirred solution of 5-bromo-2-methyl-3-(trifluoromethyl) pyridine (30 g, 124,988 mmol, 1 equiv) in 1,4-dioxane was added SeO₂ (55.47 g, 499.952 mmol, 4 equiv) at room temperature under nitrogen atmosphere. The final reaction mixture was irradiated with microwave radiation for overnight at 120° C. The resulting mixture was filtered; the filter cake was washed with DCM (3×10 mL)n The filtrate was concentrated under reduced pressure. The reaction was quenched by the addition of Water (500 mL) at room temperature. The aqueous layer was extracted with EtOAc (3×300 mL). The resulting mixture was concentrated under reduced pressure. The residue was purified by silica gel column chromatography, eluted with PE/EA (20:1) to afford 215a (25 g, 78.75%) as a yellow oil.

### 2. Synthesis of 215b

A solution of methylpropanediol (50 g, 554.803 mmol, 1 equiv) in DCM (1.5 L) was treated with TEA (168.43 g, 1664,409 mmol, 3 equiv) for 1 h at room temperature under nitrogen atmosphere followed by the addition of TsCl (317.30 g, 1664.409 mmol, 3 equiv) in three portions at room temperature. The resulting mixture was stirred for 6 h at room temperature under nitrogen atmosphere. The mixture was acidified to pH 7 with saturated NH₄Cl (aq.). The aqueous layer was extracted with EtOAc (4×300 mL). The resulting mixture was concentrated under reduced pressure. The residue was purified by silica gel column chromatography eluted with PE/EA (200:1) to afford 215b (170 g, 70.74%) as a yellow solid.

### 3. Synthesis of 215e

A solution of methyl 2-(3-nitrophenyl)acetate (50 g, 256.182 mmol, 1 equiv) in DMF (2 L) was treated with Cs₂CO₃ (417.34 g, 1280.910 mmol, 5 equiv) for 3 h at 0° C. under nitrogen atmosphere followed by the addition of 2-methylpropane-1,3-diyl bis(4-methylbenzenesulfonate) (204.17 g, 512,364 mmol, 2 equiv) in two portions at 0° C. The resulting mixture was stirred for 2 days at room temperature under nitrogen atmosphere. The mixture was acidified to pH 7 with saturated NH₄Cl (aq.). The aqueous layer was extracted with EtOAc (4×300 mL). The resulting mixture was concentrated under reduced pressure. The residue was purified by silica gel column chromatography elated with PE/EA (200:1) to afford 215c (13 g, 18.73%) as a yellow oil.

### 4. Synthesis of 215d

A solution of 215c (13 g, 52.153 mmol, 1 equiv) and hydrazine (16.71 g, 521.530 mmol, 10 equiv) in EtOH (300 mL) was stirred for 8 h at 80° C. under nitrogen atmosphere. The mixture was allowed to cool down to room temperature. The resulting mixture was diluted with water (200 mL). The aqueous layer was extracted with CH₂Cl₂ (4×l 00 mL). The resulting mixture was concentrated under reduced pressure. The residue was purified by silica gel column chromatography eluted with CH₂Cl₂/MeOH (10:1) to afford 215d (12 g, 85.84%) as a yellow solid.

### 5. Synthesis of 215e

A solution of 215d (18 g, 72.211 mmol, 1 equiv) and methyl isothiocyanate (15.84 g, 216.633 mmol, 3 equiv) in tetrahydrofuran (300 mL) was stirred for 3 h at room temperature under nitrogen atmosphere. The resulting mixture was diluted with water (200 mL). The precipitated solids were collected by filtration and washed with water (3×10 mL). The resulting solid was dried under vacuum. This resulted in 215e (19 g, 78.35%) as a white solid.

### 6. Synthesis of 215f

A solution of 215e (19 g, 58.937 mmol, 1 equiv) and NaOH (11.79 g, 294,685 mmol, 5 equiv) in H₂O (400 mL) was stirred for 8 h at room temperature under nitrogen atmosphere. The mixture was acidified to pH 6 with HCl (1 M). The precipitated solids were collected by filtration and washed with water (3×60 mL). The resulting solid was dried under vacuum. This resulted in 215f (16 g, 83.84%) as a white solid.

### 7. Synthesis of 215g

A solution of 215f (16 g, 52,568 mmol, 1 equiv) in EtOAc (200 mL) and 1H-20 (200 mL) was treated with NaNO₂ (36.27 g, 525.680 mmol, 10 equiv) at room temperature under nitrogen atmosphere followed by the addition of HNO₃ (33.12 g, 525.680 mmol, 10 equiv)/500 ml H₂O dropwise at room temperature. The resulting mixture was stirred for 6 h at room temperature under nitrogen atmosphere. The mixture to pH 7 with saturated NaHCO₃ (aq.). The precipitated solids were collected by filtration and washed with water (3×100 mL). The resulting solid was dried under vacuum. This resulted in 215g (14 g, 94.87%) as a yellow solid.

### 8. Synthesis of 215h

To a solution of 215g (14 g, 51,412 mmol, 1 equiv) in 500 mL MeOH was added Pd/C (10%, 1.6 g) under nitrogen atmosphere in a 1 L round-bottom flask. The mixture was hydrogenated at room temperature for overnight under hydrogen atmosphere using a hydrogen balloon, filtered through a Celite pad and concentrated under reduced pressure. The resulting mixture was filtered; the filter cake was washed with MeOH (3×60 mL). The filtrate was concentrated under reduced pressure. The resulting mixture was concentrated under reduced pressure. This resulted in 215h (10 g, 72.24%) as a yellow solid.

### 9. Synthesis of 215i

To a stirred solution of 215h (3 g, 12,380 mmol, 1 equiv) and 5-bromo-3-(trifluoromethyl)picolinaldehyde (4.72 g, 18.570 mmol, 1.5 equiv) in DCE (60 mL) were added NaBH(OAc)3 (5.25 g, 24.760 mmol, 2 equiv) and HOAc (0.74 g, 12.380 mmol, 1 equiv) at room temperature. The resulting mixture was stirred for overnight at room temperature. The reaction was quenched by the addition of Sat, NH₄Cl(aq.) (60 mL) at room temperature. The resulting mixture was extracted with EtOAc (3×30 mL), dried over anhydrous Na₂SO₄. After filtration, the filtrate was concentrated under reduced pressure. The residue was purified by Prep-TLC (CH₂Cl₂/MeOH 20:1) to afford PH-215i (3 g, 48.94%) as a yellow solid.

### 10. Synthesis of 215j

To a stirred solution of 21 Si (3 g, 6.220 mmol, 1 equiv) and Pyridine (2.95 g, 37.320 mmol, 6 equiv) in DCM (30 mL) were added Triphosgene (0.65 g, 2.177 mmol, 0.35 equiv) at 0° C. The resulting mixture was stirred for 20 min at room temperature under air atmosphere. The reaction was quenched by the addition of Sat. NaHCO₃ (aq.) (50 mL) at room temperature. The resulting mixture was extracted with CH₂Cl₂ (3×80 mL). The resulting mixture was concentrated under reduced pressure. The residue was purified by silica gel column chromatography eluted with CH₂Cl₂/MeOH (40:1) to afford 215j (2.8 g, 84.47%) as a yellow solid.

### 11. Synthesis of 215k

To a solution of 215j (2.2 g, 4.345 mmol, 1 equiv), TMEDA (1.01 g, 8.690 mmol, 2 equiv) in dioxane (70.00 mL) was added bis(adamantan-1-yl)(butyl)phosphane (0.31 g, 0.869 mmol, 0.2 equiv) and Pd(OAc)2 (0.10 g, 0.434 mmol, 0.1 equiv) in an autoclave. After flushing the autoclave three times with CO/H₂ (1:1), the mixture was pressurized to 10 atm with CO/H₂ (1:1) at 80° C. for overnight. The resulting mixture was concentrated under reduced pressure. The residue was purified by silica gel column chromatography eluted with CH₂Cl₂/MeOH (20:1) to afford 215k (1.4 g 70.75%) as a yellow solid.

### 12. Synthesis of 215l

Into a 50 mL 3-necked round-bottom flask were added 215k (700 mg, 1,537 mmol, 1 equiv) and 5-azaspiro [2.4] heptane hydrochloride (224.00 mg, 2.305 mmol, 1.5 equiv) at room temperature. The resulting mixture was stirred for 30 min at room temperature. To the above mixture was added HOAc (184.60 mg, 3.074 mmol, 2 equiv) and NaBH(OAc)3 (651.49 mg, 3,074 mmol, 2 equiv) at room temperature. The resulting mixture was stirred overnight at room temperature. The reaction was quenched with sat. NH₄Cl (aq.) at room temperature. The resulting mixture was extracted with CH₂Cl₂ and MeOH (10:1) (5×10 mL). The combined organic layers were concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 37% B to 67% B in 8 min; Wave Length: 220 nm; RT (min): 7.17) to afford 2151 (340 mg, 41.22%) as a yellow solid.

### 13. Synthesis of 215

The 2151 (340 mg) was purified by Chiral separation with the following conditions Column: CHIRALPAK AD-H, 2*25 cm, 5 μm; Mobile Phase A: Hex (0.5% 2M NH₃-MeOH)-HPLC, Mobile Phase B: EtOH-HPLC; Flow rate: 20 mL/min; Gradient: 25% B to 25% B in 13.5 min; Wave Length: 220/254 nm; RT1 (min): 7.70; RT2 (min): 10.08; The first peak was the product. Sample Solvent: EtOH-HPLC; Injection Volume: 0.1 mL; Number Of Runs: 34 to afford 215 (71.7 mg, 21.08%) as a yellow solid,

LC-MS: (ES, m/z): [M+H]⁺ 537. H-NMR: (400 MHz, CD₃OD, ppm): δ 0.58-0.64 (m, 4H), 31.17-1.19 (m, 3H), 31.89-1.92 (t, 2H), 32.38-2.41 (m, 2H), 32.43-2.53 (m, 1H), 32.61 (s, 2H), 32.83-2.87 (m, 2H), 33.14-3.19 (m, 2H), 33.32-3.36 (m, 3H), 33.53 (s, 2H), 37.16-7.17 (d, 2H), 37.21-7.24 (m, 1H), 37.51-7.59 (m, 2H), 37.68-7.69 (m, 2H), 38.39 (s, 1H).

### Example 216. Synthesis of Compound 216

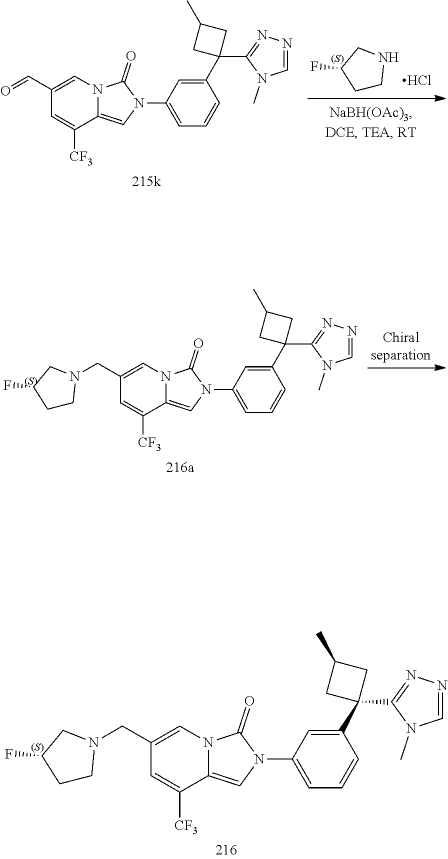

### Synthesis of 216

To a stirred solution of 215k (800 mg, 1,757 mmol, 1.00 equiv) and (3S)-3-fluoropyrrolidine hydrochloride (441.14 mg, 3.514 mmol, 2 equiv) in DCE (10 mL) were added TEA (355.50 mg, 3,514 mmol, 2 equiv) at room temperature. The mixture was stirred for 30 min at room temperature. To the above mixture was added STAB (744.56 mg, 3.514 mmol, 2 equiv) at room temperature. The resulting mixture was stirred for overnight at room temperature. The reaction was quenched by the addition of sat. NH₄Cl (aq.) (20 mL) at room temperature. The resulting mixture was extracted with CH₂Cl₂ (3×30 mL). The resulting mixture was concentrated under reduced pressure. The crude product (500 mg) was purified by Prep-HPLC with the following conditions (Column: XBridge Prep OBD C18 Column, 30*150 mm, 5 μm; Flow rate: 60 mL/min; Gradient: 32% B to 54% B in 8 min, 54% B; Wave Length: 220 nm; RT1 (min): 7.52) to afford 216 (300 mg, 31.67%) as a yellow solid

### Example 217. Synthesis of Compound 217

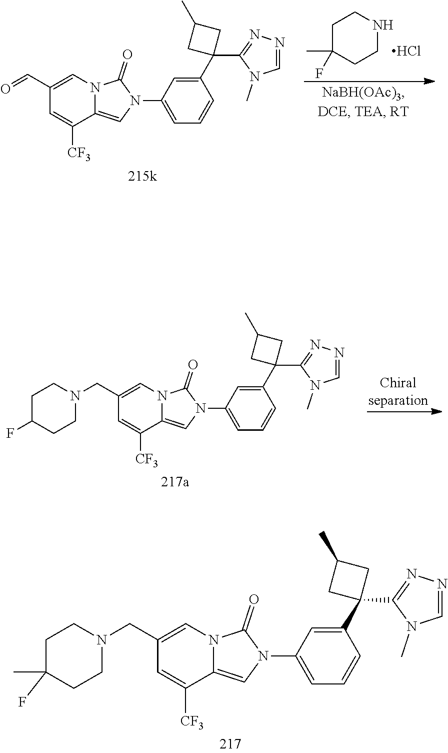

### Synthesis of 217a

To a stirred mixture of 4-fluoro-4-methylpiperidine hydrochloride (539.71 mg, 3.514 mmol, 2 equiv) and 215k (800 mg, 1.757 mmol, 1.00 equiv) in DCE (8 mL) was added TEA (533.25 mg 5.271 mmol, 3 equiv) at room temperature. The resulting mixture was stirred for 30 min at room temperature. To the above mixture was added NaBH(OAc)₃ (111684 Mg, 5.271 mmol, 3 equiv) at room temperature. The resulting mixture was stirred for additional 3 h at room temperature. The reaction was quenched by the addition of water (50 mL) at room temperature. The aqueous layer was extracted with CH₂Cl₂ (2×20 mL). The residue was purified by Prep-TLC (CH₂Cl₂/MeOH=15:1) to afford the crude product (500 mg). The crude product (500 mg) was purified by Prep-HPLC with the following conditions (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 38% B to 65% B in 8 min, 65% B; Wave Length: 220 nm; RT1 (min): 7.68) to afford 217a (400 mug, 41.97%) as a yellow solid.

### Synthesis of 217

217a (400 mg) was purified by Prep-CHIRAL-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 55% B to 55% B in 18.5 min; Wave Length: 220/254 nm; RT1 (min): 9.93; RT2 (min): 13.92; the second peak is product) to afford 217 (103.5 mg, 20.20%) as a yellow solid.

LCMS: (ES, m/z): [M+H]-557. H-NMR: (400 MHz, DMSO-d₆, ppm, δ): 1.09-1.11 (d, 3H), 1.29-1.34 (d, 3H), 1.50-1.71 (m, 1H), 1.71-1.75 (m, 3H), 2.27-2.49 (m, 5H), 2.60-2.62 (m, 2H), 3.10-3.14 (m, 2H), 3.25 (s, 3H), 7.01 (s, 1H), 7.08-7.10 (d, 1H), 7.40-7.49 (m, 1H), 7.60-7.63 (m, 1H), 7.68-7.69 (m, 1H), 7.69-7.70 (m, 1H), 8.36 (s, 1H).

### Example 218. Synthesis of Compound 218

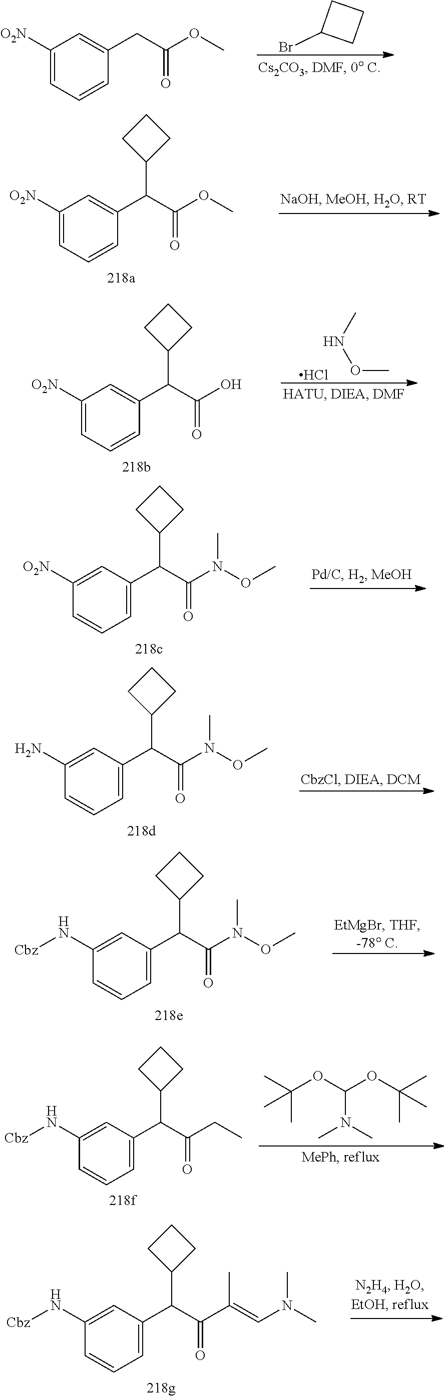

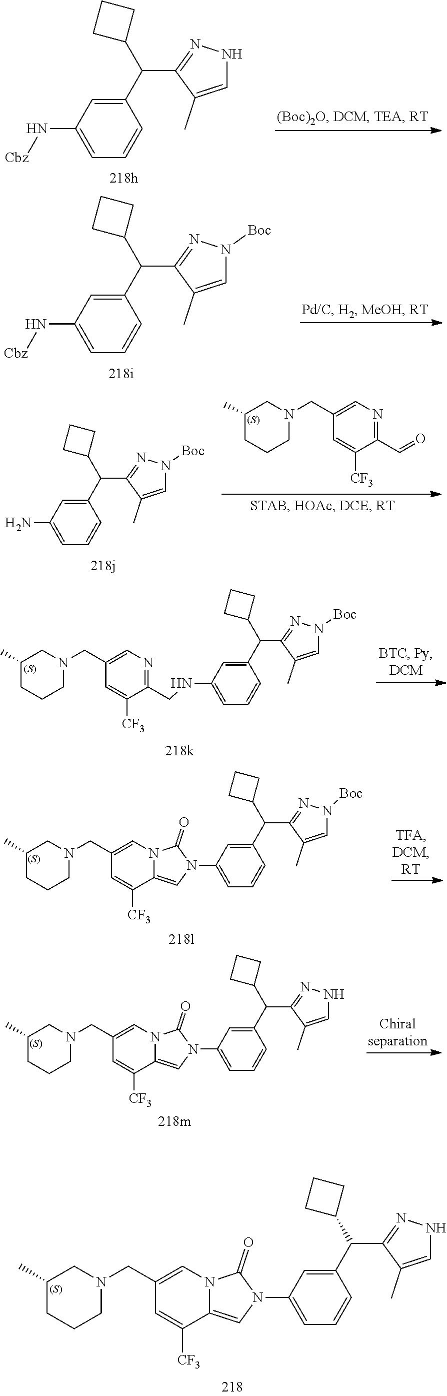

### 1. Synthesis of 218a

To a stirred mixture of methyl 2-(3-nitrophenyl)acetate (48 g, 245,934 mmol, 1 equiv) in DMF (1000 mL) was added Cs₂CO₃ (40065 g, 1229.670 mmol, 5 equiv) at 0° C. under nitrogen atmosphere. The resulting mixture was stirred for 2h at 0° C. under nitrogen atmosphere. To the above mixture was added bromocyclobutane (99.61 g, 737.802 mmol, 3 equiv) at 0° C. The resulting mixture was stirred for additional 2 days at room temperature. The resulting mixture was diluted with NH₄Cl (aq.) (3000 mL). The aqueous layer was extracted with EtOAc (2×1000 mL). The organic layers were concentrated under reduced pressure. The residue was purified by silica gel column chromatography, eluted with PE/EA (100:1) to afford 218a (45 g, 68.27%) as a white solid.

### 2, Synthesis of 218b

To a stirred solution of 218a (45 g, 180.530 mmol, 1 equiv) in MeOH (800 ml) were added NaOH (21.66 g, 541.590 mmol, 3 equiv) in H₂O (200 mL) at room temperature. The resulting mixture was stirred overnight at room temperature. The mixture was acidified to pH 5 with HCl (aq.). The aqueous layer was extracted with EtOAc (3×1000 ml). The combined organic layers were dried over anhydrous Na₂SO₄, After filtration, the filtrate was concentrated under reduced pressure. This resulted in 218b (32.1 g, 68.78%) as an off-white solid.

### 3. Synthesis of 218c

A solution of 218b (32 g, 136.032 mmol, 1 equiv) and methoxy(methyl)aminehydrochloride (17.25 g, 176.842 mmol, 1.3 equiv) in DMF (500 mL) was added HATU (56.90 g, 149.635 mmol, 1.1 equiv) and DIEA (52.74 g, 408.096 mmol, 3 equiv) at room temperature under nitrogen atmosphere. The resulting mixture was stirred overnight at room temperature. The resulting mixture was diluted with water (2000 mL). The aqueous layer was extracted with EtOAc (2×1000 mL). The organic layers were concentrated under reduced pressure. The residue was purified by silica gel column chromatography, eluted with PE/EA (10:1) to afford 218c (35 g, 85.98%) as a white solid.

### 4. Synthesis of 218d

To a solution of 218c (34 g, 122.167 mmol, 1 equiv) in MeOH (800 ML) was added Pd/C (3.4 g, 10%) under nitrogen atmosphere in a 2 L round-bottom flask. The mixture was hydrogenated at room temperature for overnight under hydrogen atmosphere using a hydrogen balloon, filtered through a Celite pad and concentrated under reduced pressure. This resulted in 218d (26 g, 77.13%) as an off-white solid.

### 5. Synthesis of 218e

To a stirred solution of 218d (26 g, 104.701 mmol, 1 equiv) and DIEA (40.60 g, 314.103 mmol, 3 equiv) in DCM (500 mL) were added CbzCl (35.72 g, 209.402 mmol, 2 equiv) at room temperature under nitrogen atmosphere. The resulting mixture was stirred overnight at room temperature. The residue was washed with water (500 mL). The aqueous layer was extracted with DCM (200 mL). The combined organic layers were dried over anhydrous Na₂SO₄. After filtration, the filtrate was concentrated under reduced pressure. The residue was purified by silica gel column chromatography, eluted with PE/EA (5:1) to afford 218e (28 g, 66.43%) as a white solid.

### 6. Synthesis of 218f

To a stirred solution of 218e (28 g, 73.210 mmol, 1 equiv) in THF (300 mL) was added EtMgBr (366.05 mL, 366.050 mmol, 5 equiv, 1M) dropwise at −78° C. under nitrogen atmosphere. The resulting mixture was stirred for 3h at room temperature under nitrogen atmosphere. The reaction was quenched with saturated NH₄Cl (aq.) (1000 mL) at room temperature. The aqueous layer was extracted with EtOAc (2×500 mL). The organic layers were concentrated under reduced pressure. The residue was purified by silica gel column chromatography, eluted with PE/EA (10:1) to afford 218f (17 g, 62.11%) as a white solid.

### 7. Synthesis of 218g

To a stirred solution of 218f (13 g, 36.990 mmol, 1 equiv) in Toluene (150 mL) was added [bis(tert-butoxy)methyl]dimethylamine (22.56 g, 110,970 mmol, 3 equiv) at room temperature under nitrogen atmosphere. The resulting mixture was stirred overnight at 110° C. The resulting mixture was concentrated under vacuum. This resulted in 218g (16.5 g, 87.78%) as a light yellow oil.

### 8. Synthesis of 218h

To a stirred solution of 218g (16.5 g, 40.588 mmol, 1 equiv) in EtOH (200 mL) was added hydrazine hydrate (98%)(20.32 g, 405.880 mmol, 10 equiv) at room temperature under nitrogen atmosphere. The resulting mixture was stirred for 6h at 80° C. The reaction was quenched with water (500 mL) at room temperature. The aqueous layer was extracted with EtOAc (2×500 mL). The resulting mixture was concentrated under vacuum. The residue was purified by silica gel column chromatography, eluted with PE/EA (2:1) to afford 218h (5.28 g, 30.49%) as a light yellow solid.

### 9. Synthesis of 218i

To a stirred solution of 218h (4.7 g, 12.518 mmol, 1 equiv) and (Boc)₂O (3.55 g, 16.273 mmol, 1.3 equiv) in DCM (50 mL) was added TEA (2.53 g, 25.036 mmol, 2 equiv) at room temperature under nitrogen atmosphere. The resulting mixture was stirred overnight at 40° C. The reaction was quenched with water (100 mL) at room temperature. The aqueous layer was extracted with DCM (2×100 mL). The organic layers were concentrated under reduced pressure. The residue was purified by silica gel column chromatography, eluted with PE/EA (10:1) to afford 218i (2.5 g, 37.79%) as an off-white solid,

### 10. Synthesis of 218j

To a solution of 218i (2.5 g, 5.257 mmol, 1 equiv) in MeOH (150 mL) was added Pd/C (0.25 g, 10%) under nitrogen atmosphere in a 500 mL round-bottom flask. The mixture was hydrogenated at room temperature for 4h under hydrogen atmosphere using a hydrogen balloon, filtered through a Celite pad and concentrated under reduced pressure. This resulted in 218j (1.7 g, 80.51%) as an off-white solid. The crude product was used in the next step directly without further purification.

### 11. Synthesis of 218k

To a stirred solution of 218j (500 mg, 1464 mmol, 1 equiv) and I-2 (419.23 mg, 1.464 mmol, 1 equiv) in DCE (8 mL) were added STAB (620.70 mg, 2,928 mmol, 2 equiv) and HOAc (87.94 mg, 1.464 mmol, 1 equiv) at room temperature under nitrogen atmosphere. The resulting mixture was stirred for 3h at room temperature. The reaction was quenched with saturated NaHCO₃ (aq.) (100 mL) at room temperature. The aqueous layer was extracted with DCM (2×50 ml). The organic layers were concentrated under reduced pressure. The residue was purified by Prep-TLC (DCM/MeOH 30:1) to afford tert-butyl 218k (510 mg 52.95%) as an off-white solid,

### 12. Synthesis of 218l

To a stirred solution of 218k (500 mg, 0.817 mmol, 1 equiv) and Pyridine (646.50 mg, 8170 mmol, 10 equiv) in DCM (10 mL) were added Triphosgene (97.02 mg 0.327 mmol, 0.4 equiv) at room temperature under nitrogen atmosphere. The resulting mixture was stirred for 10 min at room temperature. The reaction was quenched with saturated NaHCO₃ (aq.) (100 mL) at room temperature. The aqueous layer was extracted with DCM (2×50 mL). The organic layers were concentrated under reduced pressure. The residue was purified by Prep-TLC (DCM/MeOH 20:1) to afford 2181 (410 mg, 70.79%) as a yellow solid.

### 13. Synthesis of 218m

To a stirred solution of 2181 (400 mg, 0.627 mmol, 1 equiv) in DCM (5 mL) was added TEA (1 mL) at room temperature. The resulting mixture was stirred for 3h at room temperature. The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH₄CO₃), Mobile Phase B: ACN; Flow rate: 60 mL/min; Gradient: 59% B to 88% B in 8 min; Wave Length: 220 nm; RT1 (min): 7.85) to afford 218m (280 mg, 81.37%) as a yellow solid.

### 14. Synthesis of 218

218 (280 mg) was purified by Prep-Chiral-HPLC with the following conditions (Column: CHIRALPAK IE, 2*25 cm, 5 μm; Mobile Phase A: Hex (0.5% 2M NH₃-MeOH), Mobile Phase B: EtOH:DCM=1:1; Flow rate: 20 mL/min; Gradient: 15% B to 15% B in 15 min; Wave Length: 220/254 nm; RT1 (min): 10.96; RT2 (min): 12.91; the first peak is product) to afford 218 (89.5 mg, 31.80%) a: a yellow solid,

LC-MS: (ES, m/z): [M+H]⁺ 538. H-NMR: (400 MHz, CD₃OD, 3 ppm): 0.89-0.99 (m, 4H), 1.57-1.71 (m, 6H), 1.73-2.12 (m, 9H), 2.85-2.91 (m, 2H), 3.32-3.32 (m, 1H), 4.09-4.22 (m, 1H), 7.08-7.12 (m, 2H), 7.21-7.35 (m, 2H), 7.41-7.43 (m, 1H), 7.53-7.55 (m, 1H), 7.62 (s, 1H), 7.69 (s, 1H).

### Example 219. Synthesis of Compound 219

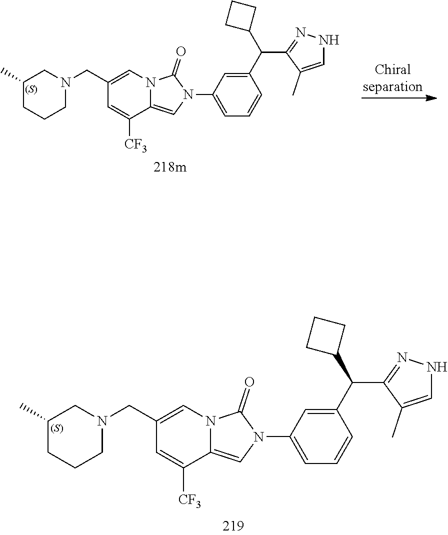

### 1. Synthesis of 219

218m (280 mg) was purified by Prep-Chiral-HPLC with the following conditions (Column: CHIRALPAK IF, 2*25 cm, 5 μm; Mobile Phase A: Hex (0.5% 2M NH-MeOH), Mobile Phase B: EtOH:DCM=1:1; Flow rate: 20 mL/min; Gradient: 15% B to 15% B in 15 min; Wave Length: 220/254 nm; RT1 (min): 10.96; RT2 (min): 12.91; the second peak is product) to afford 219 (89.5 mg, 31.80%) as a yellow solid.

LC-MS: (ES, m/z): [M+H]⁺ 538. H-NMR: (400 MHz, CD₃OD, 3 ppm): 0.89-0.99 (m, 4H), 1.57-1.71 (m, 6H), 1.73-2.12 (m, 9H), 2.85-2.91 (m, 2H), 3.32-3.32 (m, 1H), 4.09-4.22 (m, 1H), 7.08-7.12 (m, 2H), 7.21-7.35 (m, 2H), 7.41-7.43 (m, 1H), 7.53-7.55 (m, 1H), 7.62 (s, 1H), 7.69 (s, 1H).

### Example 220. Synthesis of Compound 220

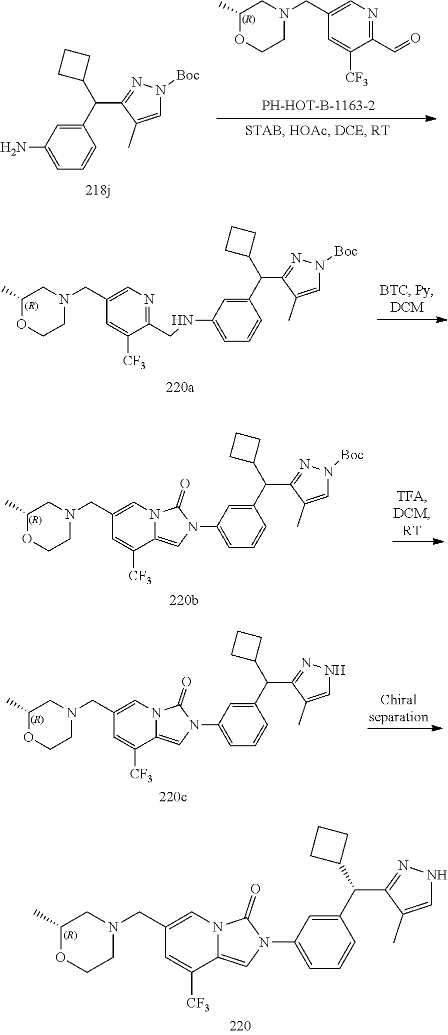

### 1. Synthesis of 220a

To a stirred solution of 218j (500 mg, 1.464 mmol, 1 equiv) and 218j (422.12 mg, 1.464 mmol, 1 equiv) in DCE (5 mL) was added HOAc (87.94 mg, 1.464 mmol, 1 equiv) and STAB (620.70 mg, 2,928 mmol, 2 equiv) at room temperature under nitrogen atmosphere. The resulting mixture was stirred for 3h at room temperature. The reaction was quenched with saturated NaHCO₃ (aq.) (100 mL) at room temperature. The aqueous layer was extracted with DCM (2×50 mL). The organic layers were concentrated under reduced pressure. The residue was purified by Prep-TLC (DCM/MeOH 30:1) to afford 220a (560 mg, 56.70%) as a white solid.

### 2. Synthesis of 220b

To a stirred solution of 220a (550 mg, 0.896 mmol, 1 equiv) and Pyridine (708.87 mg, 8.960 mmol, 10 equiv) in DCM (10 mL) were added Triphosgene (106.37 mg, 0.358 mmol, 0.4 equiv) at room temperature under nitrogen atmosphere. The resulting mixture was stirred for 10 min at room temperature. The reaction was quenched with saturated NaHCO₃ (aq.) (100 mL) at room temperature. The aqueous layer was extracted with DCM (2×50 mL). The organic layers were concentrated under reduced pressure. The residue was purified by Prep-TLC (DCM/MeOH 20:1) to afford 220b (430 mg, 67.50%) as a yellow solid.

### 3. Synthesis of 220e

To a stirred solution of 220b (420 mg, 0.657 mmol, 1 equiv) in DCM (5 mL) was added TFA (1 mL) at room temperature under nitrogen atmosphere. The resulting mixture was stirred for 3h at room temperature. The resulting mixture was concentrated under vacuum. The crude product was purified by Prep-HPLC with the following conditions (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 44% B to 74% B in 8 min, Wave Length: 220 nm; RT1 (min): 7.43) to afford 220c (300 mg, 82.99%) as a yellow solid.

### 4. Synthesis of 220

220c (300 mg) was purified by Prep-Chiral-HPLC with the following conditions (Column: CHIRALPAK IC, 2*25 cm, 5 μl; Mobile Phase A: Hex (0.5% 2M NH₃-MeOH), Mobile Phase B: EtOH:DCM=1:1; Flow rate: 20 mL/min; Gradient: 40% B to 40% B in 19 ruin; Wave Length: 220/254 nm; RT (min): 7.13; RT2 (min): 12.97; the first peak is product) to afford 220 (132.7 mg, 43.88%) as a yellow solid.

LC-MS-220 (ES, m/z): [M+H]⁺ 540. H-NMR-220 (400 MHz, CD₃OD, δ ppm): 1.13-1.15 (d, 3H), 1.61-1.77 (m, 1H), 1.77-1.90 (m, 4H), 1.90-2.05 (m, 4H), 2.05-2.10 (m, 1H), 2.10-2.22 (m, 1H), 2.74-2.83 (m, 2H), 3.24-3.32 (m, 1H), 3.34 (s, 2H), 3.63-3.71 (m, 2H), 3.84-3.88 (m, 1H), 4.03-4.18 (m, 1H), 7.08-7.10 (m, 2H), 7.27-7.29 (m, 2H), 7.43-7.45 (m, 1H), 7.53-7.55 (m, 1H), 7.61 (s, 1H), 7.70 (s, 1H).

### Example 221. Synthesis of Compound 221

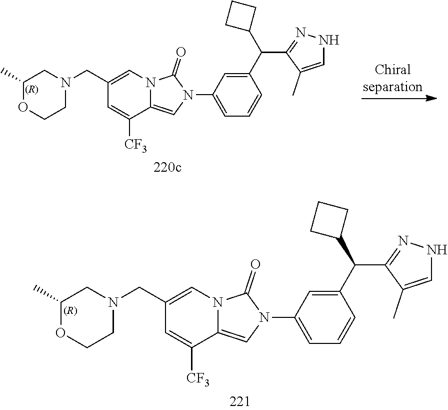

### Synthesis of 221

The 220c (300 mg) was purified by Prep-Chiral-HPLC with the following conditions (Column: CHIRALPAK IC, 2*25 cm, 5 pnm; Mobile Phase A: Hex (0.5% 2M NH₃-MeOH), Mobile Phase B: EtOH:DCM=1:1; Flow rate: 20 mL/in; Gradient: 40% B to 40% B in 19 min; Wave Length: 220/254 nm; RT1 (min): 7.13; RT2 (min): 12.97; the second peak is product) to afford 221 (139.2 mg, 45.94%) as a yellow solid.

LC-MS: (ES, m/z): [M+H]⁺ 540. H-NMR 0: (400 MHz, CD₃OD, δ ppm): 1.13-1.15 (d, 3H), 1.61-1.77 (m, 1H), 1.77-1.91 (m, 4H), 1.91-2.10 (m, 5H), 2.10-2.22 (m, 1H), 2.74-2.83 (m, 2H), 3.21-3.32 (m, 1H), 3.35 (s, 2H), 3.64-3.71 (m, 2H), 3.84-3.87 (m, 1H), 4.08-4.18 (m, 1H), 7.08-7.10 (m, 2H), 7.27-7.30 (m, 2H), 7.41-7.45 (m, 1H), 7.53-7.55 (m, 1H), 7.61 (s, 1H), 7.70 (s, 1H).

### Example 222. Synthesis of Compound 222

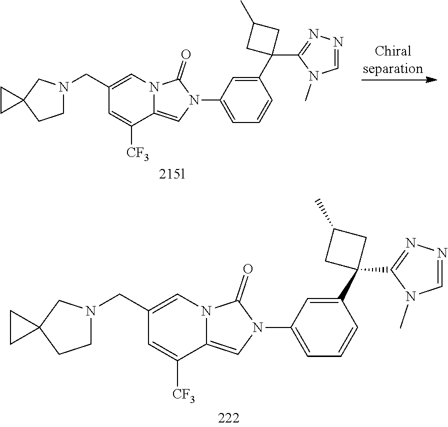 [SMILES: CC1CC(c2cccc(-n3cc4c(C(F)(F)F)cc(CN5CCC6(CC6)C5)cn4c3=O)c2)(c2nncn2C)C1 ; Cn1cnnc1[C@]1(c2cccc(-n3cc4c(C(F)(F)F)cc(CN5CCC6(CC6)C5)cn4c3=O)c2)C[C@H](C)C1]

### 1. Synthesis of 222

The 215l (340 mg) was purified by Chiral separation with the following conditions Column: CHIRALPAK AD-H, 2*25 cm, 5 μm; Mobile Phase A: Hex (0.5% 2M NT-MeOH)-HPLC, Mobile Phase B: EtOH-HPLC; Flow rate: 20 mL/min; Gradient: 25% B to 25% B in 13.5 min; Wave Length: 220/254 nm; RT1 (min): 7.70; R T2 (min): 10.08; The second peak was the product. Sample Solvent: EtOH-HPLC; Injection Volume: 0.1 mL; Number of Runs: 34 to afford 222 (131.0 ng, 38.52%) as a yellow solid.

LC-MS: (ES, m/z): [M+H]⁺ 537. H-NMR: (400 MHz, CD30D, ppm): δ 0.58-0.65 (m, 4H) δ 1.17-1.18 (d, 3H), δ 1.89-1.92 (t, 2H), δ 2.38-2.41 (m, 3H), δ 2.43-2.53 (m, 1H), δ 2.61 (m, 1H), δ 2.83-2.87 (m, 2H), δ 2.97-3.01 (m, 2H), δ 3.32-3.36 (m, 3H), δ 3.53 (s, 2H), δ 7.16-7.17 (dd, 2H), δ 7.21-7.24 (m, 1H), δ 7.51-7.59 (m, 2H), δ 7.68-7.69 (m, 2H), δ 8.39 (s, 1H).

### Example 223. Synthesis of Compound 223

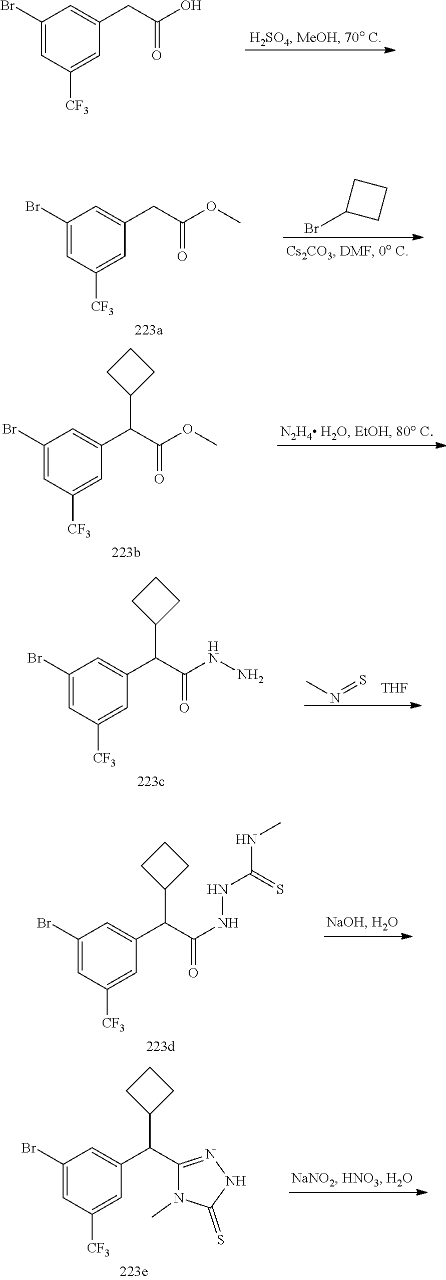

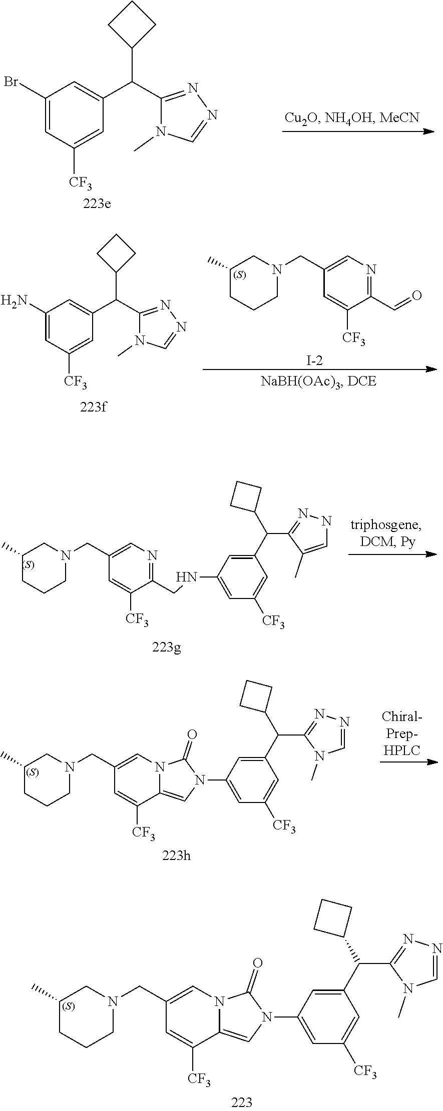

### 1. Synthesis of 223a

A solution of [3-bromo-5-(trifluromethyl)phenyl]acetic acid (8 g, 28.264 mmol, 1 equiv) in MeOH (80 mL) was added H₂SO₄ (0.28 g, 2.826 mmol, 0.1 equiv). The resulting solution was stirred for 3h at 70° C. under nitrogen atmosphere. The reaction was quenched with sat. NaHCO₃ (aq.) (100 mL) at room temperature. The aqueous layer was extracted with EtOAc (3×30 mL). The resulting mixture was concentrated under reduced pressure. This resulted in 223a (8 g, 95.28%) as colorless oil.

### 2. Synthesis of 223b

To a stirred mixture of 223a (8 g, 26.930 mmol, 1 equiv) and Cs₂CO₃ (4.74 g, 14.542 mmol, 0.54 equiv) in DMF (90 mL) was added bromocyclobutane (2.84 g, 21.005 mmol, 0.78 equiv). The resulting mixture was stirred for overnight at room temperature. The reaction was quenched with NH₄Cl (aq.) (500 ml) at room temperature. The aqueous layer was extracted with EtOAc (3×150 mL). The resulting mixture was concentrated under reduced pressure. The residue was purified by silica gel column chromatography, eluted with EA/PE (50:1) to afford 223b (9 g, 95.17%) as colorless oil.

### 3. Synthesis of 223e

A solution of 223b (8 g, 22,781 mmol, 1 equiv) and hydrazine hydrate (98%) (9.11 g, 182.248 mmol, 8 equiv) in EtOH (40 mL) was stirred for overnight at 80° C. under nitrogen atmosphere. The reaction was diluted with water (50 mL) at room temperature. The EtOH was removed under reduced pressure. The precipitated solids were collected by filtration and washed with water (2×1O mL). This resulted in 223c (7.9 g, 98.75%) as a white solid.

### 4. Synthesis of 223d

To a stirred solution of 223c (7.9 g, 22.496 mmol, 1 equiv) in tetrahydrofuran (80 mL) was added methyl isothiocyanate (3.29 g, 45.001 mmol, 2.00 equiv) at room temperature under nitrogen atmosphere. The resulting mixture was stirred for overnight at room temperature under nitrogen atmosphere. The reaction was quenched by the addition of water (50 mL) at room temperature. The THY was removed under reduced pressure. The precipitated solids were collected by filtration and washed with water (2×10 mL). This resulted in 223d (8.5 g, 89.05%) as a white solid.

### 5. Synthesis of 223e

A solution of 223d (8.45 g, 6.639 mmol, 1 equiv) in NaOH (53,112 mL, 53.112 mmol, 8 equiv, 1 M) was stirred overnight at room temperature. The reaction solution was acidified to pH 6 with HCl (1 M). The precipitated solids were collected by filtration and washed with water (2×10 mL). This resulted in 223e (6 g, 74.15%) as a white solid.

### 6. Synthesis of 223f

To a stirred mixture of 223e (6 g, 14.769 mmol, 1 equiv) and NaNO₂ (10.19 g, 147.690 mmol, 10 equiv) in EA (8 mL) and H₂O (24 mL) were added HNO₃ (147.748 mL, 147.748 mmol, 10.00 equiv, 1 M) dropwise at room temperature. The resulting mixture was stirred for overnight at room temperature. The reaction was quenched by the addition of sat. NaHCO₃ (aq.) (100 mL) at room temperature. The aqueous layer was extracted with EtOAc (4×20 mL). The resulting mixture was concentrated under reduced pressure. The residue was purified by Prep-TLC (CH₂Cl₂/MeOH 10:1) to afford 223f (1.8 g, 3257%) as a white solid.

### 7. Synthesis of 223g

To a stirred solution of 223f (3 g, 8.017 mmol, 1 equiv) in NH₄OH (15 mL) and MeCN (15 mL) were added Cu₂O (0.23 g, 1.603 mml, 0.2 equiv) at room temperature. The resulting mixture was stirred for 7h at 100° C. The reaction was diluted with water (100 mL) at room temperature. The aqueous layer was extracted with EtOAc (5×40 mL). The resulting mixture was concentrated under reduced pressure. This resulted in 223g (1.6 g, 64.31%) as a white solid.

### 8. Synthesis of 223h

To a stirred mixture of 223g (600 mg, 1.933 mmol, 1 equiv) and 5-{[(3S)-3-methylpiperidin-1l-yl]methyl}-3-(trifluoromethyl)pyridine-2-carbaldehyde (1107109 mg, 3.866 mmol, 2 equiv) in DCE (7 mL) were added HOAC (116.11 mg, 1.933 mmol, 1 equiv) and NaBH(OAc)₃ (819.56 mg, 3.866 mmol, 2 equiv) at room temperature under nitrogen atmosphere. The resulting mixture was stirred for overnight at room temperature under nitrogen atmosphere. The reaction was quenched with water (50 mL) at room temperature. The aqueous layer was extracted with EtOAc (3×50 mL). The resulting mixture was concentrated under reduced pressure. The residue was purified by Prep-TLC (DCM/MeOH 20:1) to afford 223h (820 mg, 73.04%) as yellow solid.

### 9. Synthesis of 223i

To a stirred solution of 223h (820 mg, 1,412 mmol, 1 equiv) and Pyridine (670.27 mg, 8.472 mmol, 6 equiv) in DCM (24 mL) was added triphosgene (146.68 mg, 0.494 mmol, 0.35 equiv) at room temperature under nitrogen atmosphere. The resulting mixture was stirred for 30 min at room temperature under nitrogen atmosphere. The reaction was quenched with NaHCO₃ (aq.) (50 mL) at room temperature. The aqueous layer was extracted with EtOAc (3×30 mL). The resulting mixture was concentrated under reduced pressure. The residue was purified by Prep-TLC (CH₂Cl₂/MeOH 10:1) to afford 223i (480 mg, 56.03%) as a yellow solid

### 10. Synthesis of 223

223i (480 mg) was separated by Chiral-Prep-HPLC with the following conditions (Column: CHIRALPAK 10, 2*25 cm, 5 μm; Mobile Phase A: Hex (0.5% 2M NH₃-MeOH), Mobile Phase B: EtOH:DCM=1:1; Flow rate: 20 mL/min; Gradient: 30% B to 30% B in 7.5 min; Wave Length: 220/254 nm; RT1 (min): 4.52; RT2 (min): 5.64; Sample Solvent: EtOH: DCM=1:1; The first peak was the product). This resulted in 223 (195.1 mg, 40.65%) as a yellow solid.

LC-MS-223 (ES, m/z): [M+H]⁺ 607. H-NMR-223 (400 MHz, DMSO, δ ppm): 0.82-0.90 (m, 4H), 1.31-1.42 (m, 1H), 1.59-1.76 (m, 2H), 1.78-1.84 (m, 2H), 1.85-1.99 (m, 6H), 2.01-2.12 (m, 1H), 2.52-2.74 (m, 2H), 3.21-3.25 (m, 3H), 3.49 (s, 3H), 4.44-4.46 (d, 1H), 7.03 (s, 1H), 7.55-7.59 (d, 2H), 7.65 (s, 1H), 8.14-8.18 (d, 2H), 8.36 (s, 1H).

### Example 224. Synthesis of Compound 224

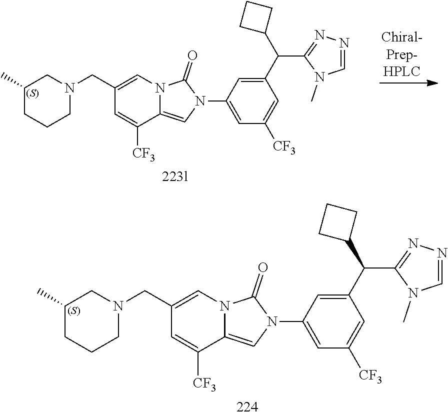

### Synthesis of Compound 224

223i (480 mg) was separated by Chiral-Prep-HPLC with the following conditions (Column: CHIRALPAK IG 2*25 cm, 5 um; Mobile Phase A: Hex (0.5% 2M NH₃-MeOH), Mobile Phase B: EtOH:DCM=1:1; Plow rate: 20 mL/min; Gradient: 30% B to 30% B in 7.5 min; Wave Length: 220/254 nm; RT1 (min): 4.52; RT2 (min): 5.64; Sample Solvent: EtOH: DCM=1:1; The second peak was the product). This resulted in 2224 (185.3 mg, 38.60%) as a yellow solid.

LC-MS-224 (ES, m/z): [M+H]⁺ 607. H-NMR-224 (400 MHz, DMSO, δ ppm): 0.82-0.90 (m, 4H), 1.31-1.42 (m, 1H), 1.67-1.78 (m, 4H), 1.80-1.99 (m, 6H), 2.05-2.08 (m, 1H), 2.60-2.74 (m, 2H), 3.21-3.25 (m, 3H), 3.49 (s, 3H), 4.44-4.46 (d, 1H), 7.04 (s, 1H), 7.55-7.59 (d, 2H), 7.65 (s, 1H), 8.14-8.18 (d, 2H), 8.36 (s, 1H).

### Example 225. Synthesis of Compound 225

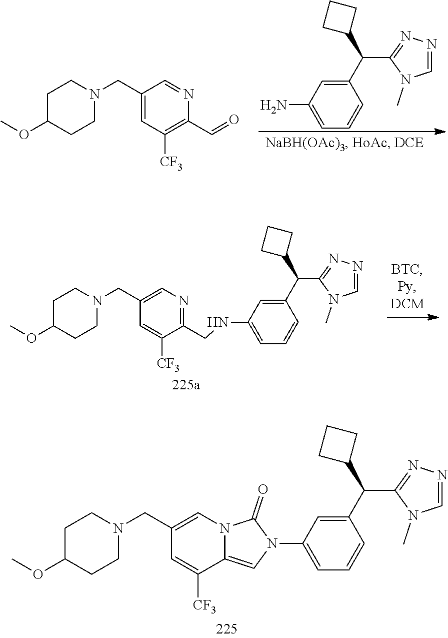 [SMILES: COC1CCN(Cc2cnc(C=O)c(C(F)(F)F)c2)CC1 ; Cn1cnnc1[C@@H](c1cccc(N)c1)C1CCC1 ; COC1CCN(Cc2cnc(CNc3cccc([C@H](c4nncn4C)C4CCC4)c3)c(C(F)(F)F)c2)CC1 ; COC1CCN(Cc2cc(C(F)(F)F)c3cn(-c4cccc([C@H](c5nncn5C)C5CCC5)c4)c(=O)n3c2)CC1]

### Synthesis of 225a

To a stirred solution of 235c (299.40 mg, 0.990 mmol, 1.2 equiv) and (R)-3-(cyclobutyl(4-methyl-4H-1,2,4-triazol-3-yl)methyl)aniline (200 mg, 0.825 mmol, 1 equiv) in DCE (3 mL) were added STAB (349.84 mg, 1.650 mmol, 2 equiv) and HOAc (49.56 mg, 0.825 mmol, 1 equiv) at room temperature. The resulting mixture was stirred for overnight at room temperature. The reaction was quenched by the addition of Sat. NH₄Cl(aq.) (10 mL) at room temperature. The resulting mixture was extracted with CH₂Cl₂ (3×10 mL). The resulting mixture was concentrated under reduced pressure. The residue was purified by Prep-TLC (CH₂Cl₂/MeOH 20:1) to afford 225a (300 mg, 65.32%) as a yellow solid.

### Synthesis of 225

To a stirred solution of 225a (200 mg, 0.378 mmol, 1 equiv) and pyridine (39.30 mg, 0.132 mmol, 0.35 equiv) in DCM (3 mL) were added Triphosgene (39.30 mg, 0.132 mmol, 0.35 equiv) at 0° C. The resulting mixture was stirred for 20 min at room temperature. The reaction was quenched by the addition of sat. NaHCO₃ (aq.) (10 mL) at room temperature. The resulting mixture was extracted with CH₂Cl, (3×10 mL). The resulting mixture was concentrated under reduced pressure. The residue was purified by reverse flash chromatography with the following conditions: C18 silica gel 120 g; mobile phase, MeCN in 0.1% NH₄HCO₃ water, 20% to 40% gradient in 10 min; detector, UV 254 nm. This resulted in 225 (96.6 mg, 45.71%) as a yellow solid.

LC-MS-PH-225 (ES, m/z): [M+H]⁺: 555. H-NMR-PH-225 (400 MHz, DMSO-d6, ppm): δ 1.32-1.50 (m, 2H), δ 1.62-1.85 (m, 7H), δ 2.03-2.21 (m, 3H), δ 2.63-2.73 (m, 2H), δ 3.18-3.25 (m, 7H), δ 3.45 (s, 3H), δ 4.25-4.27 (d, 1H), δ 7 01 (s, 1H), δ 7.18-7.20 (d, 1H), δ 731 (s, 1H), δ 7.44-7.48 (m, 1H), δ 7.60-7.62 (d, 2H), δ 7.70 (s, 1H), δ 8.33 (s, 1H),

### Example 226. Synthesis of Compound 226

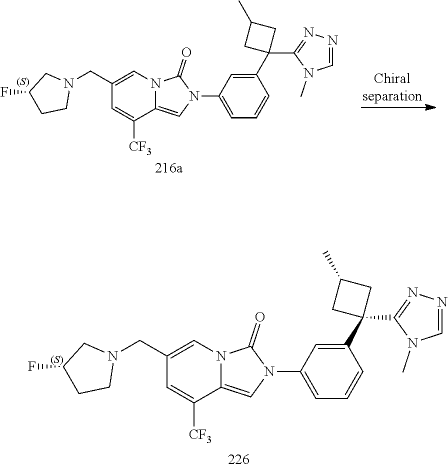

### Synthesis of 226

The 216a (300 mg) was purified by Chiral separation with the following conditions (Column: CHIRALPAK IC, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 45% B to 45% B in 20 min; Wave Length: 220/254 inn; RT1 (min): 13.77; RT2 (min): 16.98; The second peak was the product. Sample Solvent: EtOH:DCM=1:1-HPLC; Injection Volume: 0.2 mL; Number of Runs: 15). This resulted in 226 (156.8 mg, 51.64%) as a yellow solid.

LC-MS: (ES, m/z): [M+H]+: 529. H-NMR: (400 MHz, DMSO-d6, ppm): δ 1.12-1.13 (m, 3H), δ 1.80-1.97 (m, 1H), δ 2.04-2.25 (m, 1H), δ 2.28-2.33 (m, 1H), δ 2.51 (s, 3H), δ 2.63-2.65 (d, 1H), δ 2.81-2.95 (m, 2H), δ 3.21 (s, 3H), δ 3.33 (s, 1H), δ 3.45 (s, 1H), δ 5.12-5.31 (d, 1H), δ 7.02 (s, 1H), δ 7.23-7.25 (d, 1H), δ 7.41 (s, 1H), δ 7.43-7.46 (m, 1H), δ 7.62-7.63 (d, 1H), δ 7.68 (s, 1H), δ 7.82 (s, 1H), δ 8.31 (s, 1H).

### Example 227. Synthesis of Compound 227

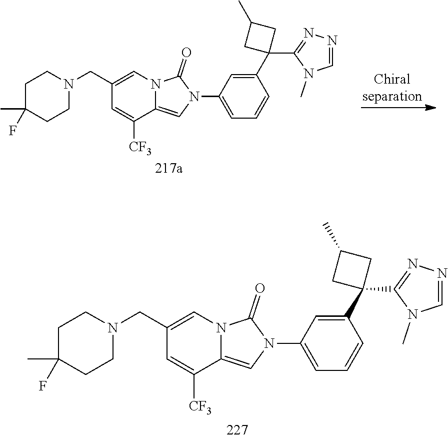 [SMILES: CC1CC(c2cccc(-n3cc4c(C(F)(F)F)cc(CN5CCC(C)(F)CC5)cn4c3=O)c2)(c2nncn2C)C1 ; Cn1cnnc1[C@]1(c2cccc(-n3cc4c(C(F)(F)F)cc(CN5CCC(C)(F)CC5)cn4c3=O)c2)C[C@H](C)C1]

217a (400 mg) was separated by Prep-CHIRAL-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 55% B to 55% B in 18.5 min; Wave Length: 220/254 nm; RT1 (min): 9.93; RT2 (min): 13.92; the first peak is product) to afford 227 (210.3 mg, 40.63%) as a yellow solid.

LCMS-227 (ES, m/z): [M+H]⁺ 557. H-NMR-227 (400 MHz, DMSO-d₆, ppm, δ): 1.09 (s, 3H), 1.29-1.34 (d, 3H), 1.61-1.65 (m, 1H), 1.65-1.73 (m, 3H), 2.24-2.29 (m, 2H), 2.51-2.54 (m, 2H), 2.54-2.62 (m, 2H), 2.88 (s, 2H), 3.21 (s, 3H), 7.01 (s, 1H), 7.22-7.26 (d, 1H), 7.37 (s, 1H), 7.48-7.52 (t, 1H), 7.63-7.68 (m, 1H), 7.84 (s, 1H), 8.29 (s, 1H)

### Example 228. Synthesis of Compound 228

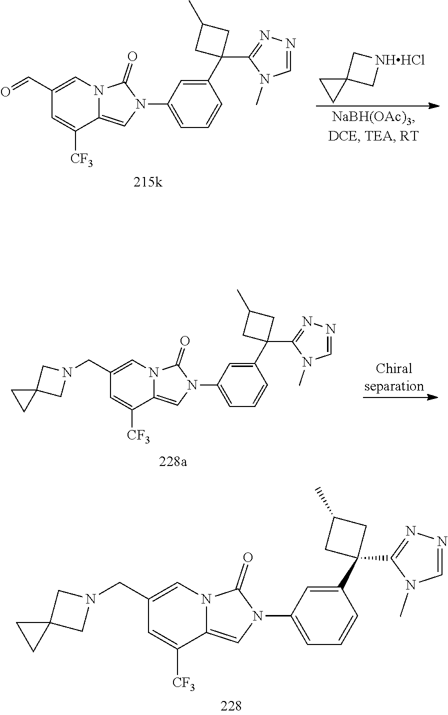

### Synthesis of 228a

To a stirred solution of 215k (800 mg, 1.757 mmol, 1.00 equiv) and 5-azaspiro[2,3]hexane hydrochloride (420.13 mg, 3.514 mmol, 2 equiv) in DCE (10 mL) were added TEA. (355.50 mg, 3,514 mmol, 2 equiv) at room temperature. The mixture was stirred for 30 min at room temperature. To the above mixture was added STAB (744.56 mg, 3.514 mmol, 2 equiv) at room temperature. The resulting mixture was stirred for overnight at room temperature. The reaction was quenched by the addition of sat. NH4Cl (aq.) (20 mL) at room temperature. The resulting mixture was extracted with CH2Cl2 (3×30 mL). The resulting mixture was concentrated under reduced pressure. The crude product (400 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 60% B in 8 min, 60% B; Wave Length: 220 nm; RT1 (min): 7.36/7.63) to afford 228a (320 mg, 34.16%) as a yellow solid.

### Example 229. Synthesis of Compound 229

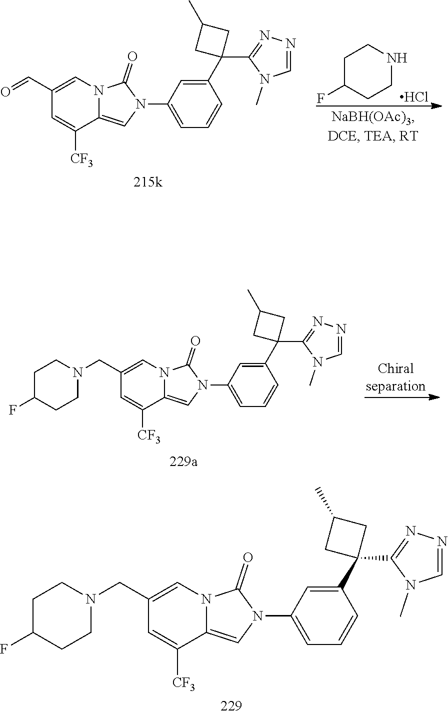 [SMILES: CC1CC(c2cccc(-n3cc4c(C(F)(F)F)cc(C=O)cn4c3=O)c2)(c2nncn2C)C1 ; FC1CCNCC1 ; Cl ; CC1CC(c2cccc(-n3cc4c(C(F)(F)F)cc(CN5CCC(F)CC5)cn4c3=O)c2)(c2nncn2C)C1 ; Cn1cnnc1[C@]1(c2cccc(-n3cc4c(C(F)(F)F)cc(CN5CCC(F)CC5)cn4c3=O)c2)C[C@H](C)C1]

### Synthesis of 229a

To a stirred solution of 215k (800 mg, 1.757 mmol, 1 equiv) and 4-fluoropiperidine hydrochloride (490.42 mg, 3.514 mmol, 2 equiv) in DCE (8 mL) was added TEA (355.49 mg, 3.514 mmol, 2 equiv) at room temperature under nitrogen atmosphere. The resulting mixture was stirred for 1h at room temperature. To the above mixture was added NaBH(OAc)3 (744.57 mg 3-514 mmol, 2 equiv) at room temperature. The resulting mixture was stirred overnight at room temperature. The reaction was quenched with saturated NH₄Cl (aq.) (50 mL) at room temperature. The aqueous layer was extracted with DCM (2×20 mL). The organic layers were concentrated under reduced pressure. The residue was purified by Prep-TLC (DCM/MeOH 20:1) to afford the crude product. The crude product (500 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 17% B to 30% B in 7 min; Wave Length: 254; 220 nm; RT1 (min): 5.27) to afford 229a (400 mg, 41.13%) as a yellow solid.

### Example 230. Synthesis of Compound 230

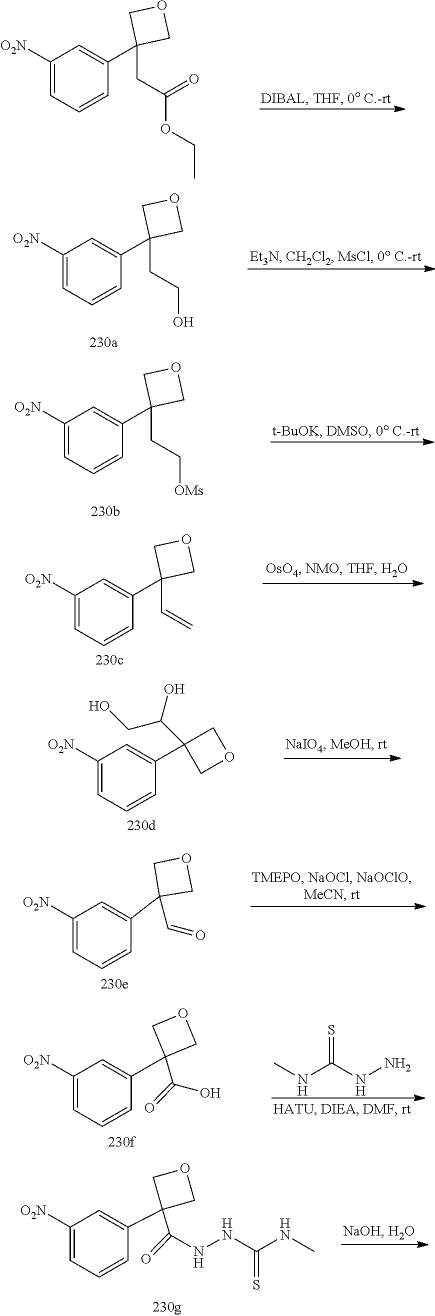

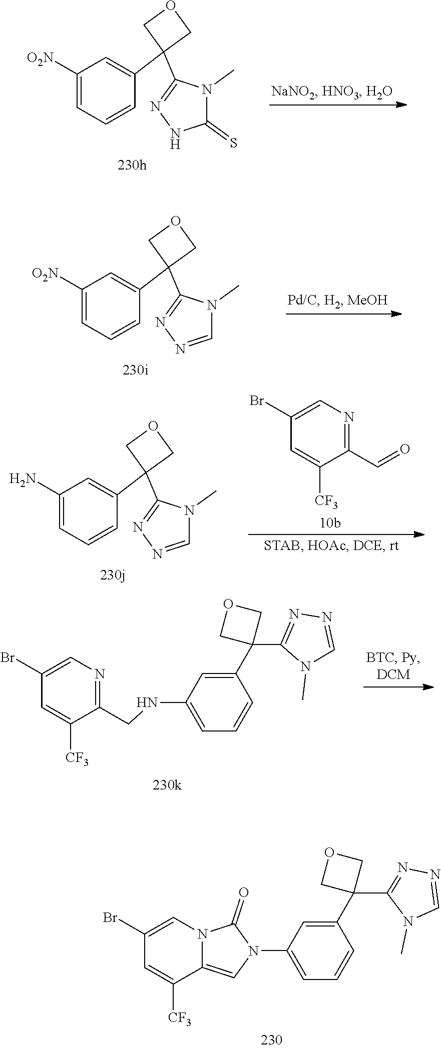 [SMILES: Cn1c(C2(c3cccc([N+](=O)[O-])c3)COC2)n[nH]c1=S ; Cn1cnnc1C1(c2cccc([N+](=O)[O-])c2)COC1 ; Cn1cnnc1C1(c2cccc(N)c2)COC1 ; O=Cc1ncc(Br)cc1C(F)(F)F ; Cn1cnnc1C1(c2cccc(NCc3ncc(Br)cc3C(F)(F)F)c2)COC1 ; Cn1cnnc1C1(c2cccc(-n3cc4c(C(F)(F)F)cc(Br)cn4c3=O)c2)COC1]

### 1. Synthesis of 230a

To a stirred solution of ethyl 2-(3-(3-nitrophenyl)oxetan-3-Yl)acetate (10 g, 37.698 mmol, 1 equiv) in THF (100 mL) was added DIBAl-H (37.70 mL, 75.396 mmol, 2 equiv) at 0° C. under nitrogen atmosphere. The resulting mixture was stirred overnight at room temperature. The reaction was quenched with saturated NH4Cl (aq.) (500 mL) at room temperature. The aqueous layer was extracted with EtOAc (2×200 mL). The organic layers were concentrated under reduced pressure. The residue was purified by silica gel column chromatography, eluted with PB/EA (5:1) to afford 230a (4 g, 43.25%) as a light yellow oil.

### 2. Synthesis of 230b

To a stirred solution of 230a (4 g, 17.919 mmol, 1 equiv) and MsCl (3.08 g, 26.879 mmol, 1.5 equiv) in DCM (100 mL) was added TEA. (5.44 g, 53.757 mmol, 3 equiv) at room temperature under nitrogen atmosphere. The resulting mixture was stirred overnight at room temperature. The reaction was quenched with water (200 mL) at room temperature. The resulting mixture was extracted with DCM (2×100 mL). The combined organic layers were dried over anhydrous Na2SO4, After filtration, the filtrate was concentrated under reduced pressure. This resulted in 230b (4 g, 66.68%) as a yellow oil.

### 3. Synthesis of 230c

To a stirred solution of 230b (4 g, 13.275 mmol, 1 equiv) in DMSO (100 mL) was added t-BuOK (4.47 g, 39.825 mmol, 3 equiv) at 0° C. under nitrogen atmosphere. The resulting mixture was stirred overnight at room temperature. The reaction was quenched with saturated NH4Cl (aq.) (300 mL) at room temperature. The aqueous layer was extracted with EtOAc (2×100 mL). The organic layers were concentrated under reduced pressure. The residue was purified by silica gel column chromatography, eluted with PE/BA (10:1) to afford 230c (2 g, 71.21%) as an off-white solid.

### 4. Synthesis of 230d

To a stirred solution of 230c (1.5 g, 7.309 mmol, 1 equiv) and OsO₄ (0.19 g, 0.731 mmol, 0.1 equiv) in THF (20 mL) was added NMO (2.57 g, 21.927 mmoL, 3 equiv) in H2O (10 mL) at 0° C. The resulting mixture was stirred overnight at room temperature. The reaction was quenched with water (100 mL) at room temperature. The aqueous layer was extracted with EtOAc (2×100 mL). The organic layers were concentrated under reduced pressure. This resulted in 230d (1.5 g, 78.92%) as a brown oil.

### 5. Synthesis of 230e

To a stirred solution of 230d (1.5 g, 6,270 mmol, 1 equiv) in MeOH (30 mL) was added NaIO4 (2.68 g, 12.540 mmol, 2 equiv) at room temperature. The resulting mixture was stirred overnight at room temperature. The reaction was quenched with water (100 mL) at room temperature. The aqueous layer was extracted with EtOAc (2×50 mL). The organic layers were concentrated under reduced pressure. The residue was purified by Prep-TLC (PE/EA 2:1) to afford 230e (750 mg, 51.96%) as an off-white solid.

### 6. Synthesis of 230f

To a stirred solution of 230e (400 mg, 1.931 mmol, 1 equiv) and TEMPO (30.17 mg, 0.193 mmol, 0.1 equiv) in MeCN (5 mL)/H2O (2 mL) was added NaOCl (114.97 mg, 1.545 mmol, 0.8 equiv) and NaOClO (698.44 mg, 7.724 mmol, 4 equiv) at room temperature. The resulting mixture was stirred overnight at room temperature. The residue was purified by reverse flash chromatography with the following conditions: column, C18 silica gel; mobile phase, MeCN in water (0.5% FA), 30% to 50% gradient in 10 min; detector, UV 254 nm. This resulted in 230f (200 mg, 45.02%) as an off-white solid.

### 7. Synthesis of 230g

To a stirred solution of 230f (200 mg, 0,896 mmol, 1 equiv) and 1-amino-3-methylthiourea (141.35 mg, 1.344 mmol, 1.5 equiv) in DMF (3 mL) was added HATU (442.96 mg, 1.165 mmol, 1.3 equiv) and DIEA (231.64 mg, 1.792 mmol, 2 equiv) at room temperature. The resulting mixture was stirred overnight at room temperature. The resulting mixture was diluted with water (10 mL). The precipitated solids were collected by filtration. This resulted in 230g (180 mg, 58.25%) as an off-white solid.

### 8. Synthesis of 230h

To a stirred solution of NaOH (92.80 mg, 2,320 mmol, 4 equiv) in H2O (3 mL) was added 230g (180 mg, 0,580 mmol, 1 equiv) at room temperature. The resulting mixture was stirred for overnight at room temperature. The mixture was acidified to pH 6 with HCl (aq.). The aqueous layer was extracted with DCM (3×10 mL). The resulting mixture was concentrated under vacuum. This resulted in 230h (120 mg, 65.11%) as an off-white solid.

### 9. Synthesis of 230i

To a stirred solution of 230h (120 mg, 0.411 mmol, 1 equiv) and NaNO2 (283.24 mg, 4.110 mmol, 10 equiv) in H2O (3 mL) was added HNO3 (4.11 mL, 4.110 mmol, 10 equiv, 1M) at 0° C. The resulting mixture was stirred overnight at room temperature. The reaction was quenched with saturated NaHCO3 (aq.) (50 mL) at room temperature. The aqueous layer was extracted with DCM (3×10 mL). The organic layers were concentrated under reduced pressure. The residue was purified by Prep-TLC (DCM/MeOH 20:1) to afford 230i (80 mg, 68.89%) as an off-white solid.

### 10. Synthesis of 230j

To a solution of 230i (75 mg, 0.288 mmol, 1 equiv) in MeOH (10 ml) was added Pd/C (7.5 mg, 10%) under nitrogen atmosphere in a 50 mL round-bottom flask. The mixture was hydrogenated at room temperature for 2h under hydrogen atmosphere using a hydrogen balloon, filtered through a Celite pad and concentrated under reduced pressure. This resulted in 230j (50 mg, 67.81%) as an off-white solid. The crude product was used in the next step directly without further purification.

### 11. Synthesis of 230k

To a stirred solution of 230i (50 mg, 0.217 mmol, 1 equiv) and 10b (60.67 mg, 0.239 mmol, 1.1 equiv) in DCE (2 mL) were added HOAc (1304 mg, 0.217 mmol, 1 equiv) and STAB (92.04 mg, 0.434 mmol, 2 equiv) at room temperature under nitrogen atmosphere. The resulting mixture was stirred overnight at room temperature. The resulting mixture was concentrated under vacuum. The residue was purified by Prep-TLC (DCM/MeOH 15:1) to afford 230k (50 mg, 45.73%) as an off-white solid.

### 12. Synthesis of 230

To a stirred solution of 230k (50 mg, 0.107 mmol, 1 equiv) and Pyridine (126.69 mg, 1,605 mmol, 15 equiv) in DCM (3 mL) were added Triphosgene (19.01 mg, 0.064 mmol, 0.6 equiv) at room temperature under nitrogen atmosphere. The resulting mixture was stirred for 10 min at room temperature. The reaction was quenched with saturated NaHCO3 (aq.) (30 mL) at room temperature. The aqueous layer was extracted with DCM (2×10 mL). The organic layers were concentrated under reduced pressure. The resulting mixture was concentrated under vacuum. The residue was purified by reverse flash chromatography with the following conditions: column, C18 silica gel; mobile phase A: water (10 mmol/L NH4HCO3), B: MeCN, 50% B to 60% B gradient in 10 min; detector, UV 254 n. This resulted in 230 (31.3 mg, 58.71%) as a yellow solid.

LC-MS-230 (ES, m/z): [M+H]⁺ 494. H-NMR-230 (400 MHz, DMSO-d6, δ ppm): 3.25 (s, 3H), 5.12-5.14 (d, 2H), 5.38-5.40 (d, 2H), 7.17-7.20 (m, 2H), 7.54-7.58 (m, 2H), 7.76-7.80 (m, 2H), 8.04 (s, 1H), 8.49 (s, 1H).

### Example 231. Synthesis of Compound 231

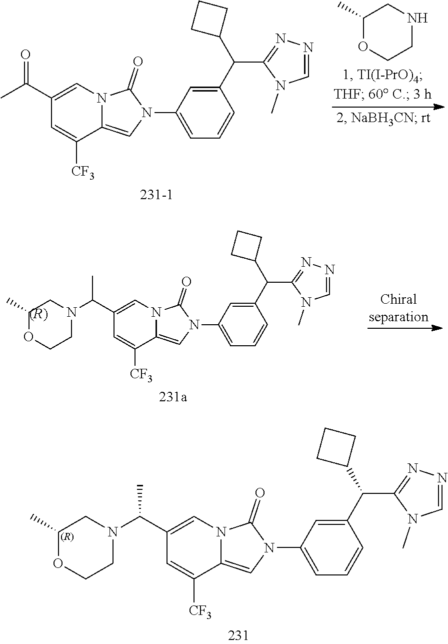

### 1. Synthesis of 231a

To a stirred mixture of 231-1 (4 g, 8.520 mmol, 1 equiv) and (2R)-2-methylmorpholine (2.59 g, 25,560 mmol, 3 equiv) in THF (40 mL) was added Ti(Oi-Pr)4 (9.69 g, 34.080 mmol, 4 equiv) at room temperature. The resulting mixture was stirred for 3h at 60° C. To the above mixture was added NaBH₃CN (1.61 g, 25,560 mmol, 3 equiv) at room temperature. The resulting mixture was stirred for additional overnight at room temperature. The reaction was quenched by the addition of NH₄Cl (aq.) (40 mL) at room temperature. The resulting mixture was diluted with water (120 mL). The aqueous layer was extracted with CH₂Cl₂/MeOH (10:1) (3×100 mL). The resulting mixture was concentrated under reduced pressure. The residue was purified by silica gel column chromatography, eluted with Cl₂Cl₂/MeOH (60:1) to afford 231a (1.2 g, crude) as a yellow solid. The crude product was purified by Prep-TLC (CH₂Cl₂/MeOH 12:1) to afford 231a (1 g, crude) as a yellow solid. The crude product was purified by reverse flash chromatography with the following conditions: column, silica gel; mobile phase, MeCN in water (10 mmol/L NH₄HCO₃), 2% to 100% gradient in 40 min; detector, UV 254 nm to afford 231a (850 mg, 16.55%) as a yellow solid.

### 2. Synthesis of 231

231a (830 mg) was purified by Chiral separation with the following conditions (Column: CHIRAL ART Cellulose-SB, 3*25 cm, 5 μm; Mobile Phase A: CO₂, Mobile Phase B: MeOH (0.1% 2M NH₃-MeOH); Flow rate: 100 mL/min; Gradient: isocratic 20% B; Column Temperature (° C.): 35; Back Pressure (bar): 100; Wave Length: 220 nm; RT1 (min): 572; RT2 (min): 6.96; RT3 (min): 7.75; RT4 (min): 9.80; first peak is product) to afford 231 (174.1 mg, 19.93%) as a yellow solid.

LCMS: (ES, m/z): [M+H]⁺ 555. H-NMR: H NMR (400 MHz, DMSO, ppm): δ 1.00-1.00-1.02 (d, 3H), δ 1.24-1.27 (d, 3H), δ 1.76-1.81 (m, 6H), δ 2.01-2.12 (m, 2H), δ 2.69-2.80 (m, 2H), δ 3.17-3.28 (m, 1H), δ 3.39-3.48 (m, 6H), δ 3.71-4.28 (m, 2H), δ 7.05 (s, 1H), δ 7.11-7.24 (d, 1H), δ 7.32 (s, 1H), δ 7.40-7.55 (t, 1H), δ 7.62 (s, 1H), δ 7.65-7.80 (m, 2H), δ 8.33 (s, 1H).

### Example 232. Synthesis of Compound 232

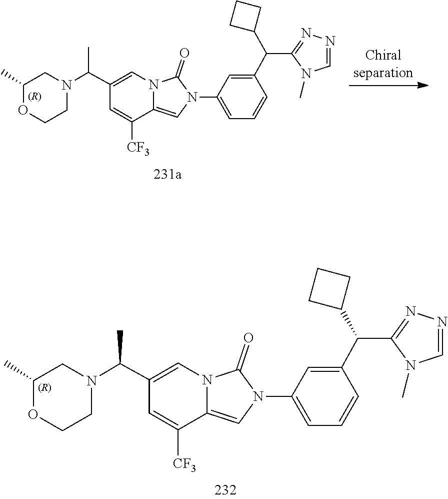

231a (830 mg) was purified by Chiral separation with the following conditions (Column: CHIRAL ART Cellulose-SB, 3*25 cm, 5 μm; Mobile Phase A: C02, Mobile Phase B: MeOH (0.1% 2M NH-MeOH); Flow rate: 100 mL/min; Gradient: isocratic 20% B; Column Temperature (° C.): 35; Back Pressure (bar): 100; Wave Length: 220 nm; T1 (min): 5.72; RT2 (min): 6.96; RT3 (min): 7.75; RT4 (min): 9.80; second peak is product) to afford 232 (122.5 mg, 14.61%) as a yellow solid.

LCMS-232 (ES, m/z): [M+H]⁺ 555. H-NMR-232 H NMR (400 MHz, DMSO, ppm): δ 1.00-1.06 (d, 3H), δ 1.25-1.27 (d, 3H), δ 1.72-1.81 (m, 6H), δ 2.01-2.12 (m, 2H), δ 2.59-2.63 (m, 1H), δ 2.77-2.80 (d, 1H), δ 3.17-3.28 (m, 1H), δ 3.36-3.50 (m, 6H), δ 3.71-3.74 (d, 1H), δ 4.24-4.28 (d, 1H), δ 7.05 (s, 1H), δ 7.18-7.24 (d, 1H), δ 7.32 (s, 1H), δ 7.41-7.48 (t, 1H), δ 7.62 (s, 1H), δ 7.66-7.70 (d, 1H), δ 7.73 (s, 1H), δ 8.33 (s, 1H).

### Example 233. Synthesis of Compound 233

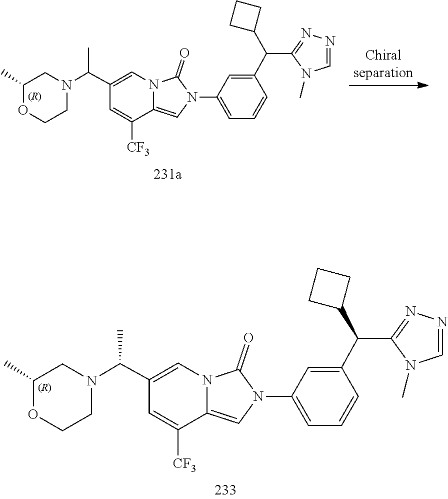

### Synthesis of 233

231a (830 mg) was purified by Chiral separation with the following conditions (Column: CHIRAL ART Cellulose-SB, 3*25 cm, 5 um; Mobile Phase A: C02, Mobile Phase B: MeOH (0.1% 2M NH3-MeOH); Flow rate: 100 mL/min; Gradient: isocratic 20% B; Column Temperature (° C.): 35; Back Pressure (bar): 100; Wave Length: 220 nm; RT1 (min): 5.72; RT2 (min): 6.96; RT3 (min): 7.75; RT4 (min): 9.80; third peak is product) to afford 233 (101.8 mg, 1214%) as a yellow solid.

LCMS-233 (ES, m/z): [M+H]⁺ 555. H-NMR-233 H NMR (400 MHz, DMSO, ppm): δ 0.96-1.05 (d, 3H), δ 1.24-1.27 (d, 3H), δ 1.72-1.81 (m, 6H), δ 2.03-2.12 (m, 2H), 62.65-2.74 (m, 2H), δ 3.17-3.25 (m, 1H), δ 3.39-3.50 (m, 6H), δ 3.75-3.80 (d, 1H), δ 4.24-4.28 (d, 1H), δ 7.05 (s, 1H), δ 7.18-7.24 (d, 1H), δ 7.31 (s, 1H), δ 7.41-7.48 (t, 1H), δ 7.62 (s, 1H), δ 7.66-7.70 (d, 1H), δ 7.73 (s, 1H), δ 8.33 (s, 1H).

### Example 234. Synthesis of Compound 234

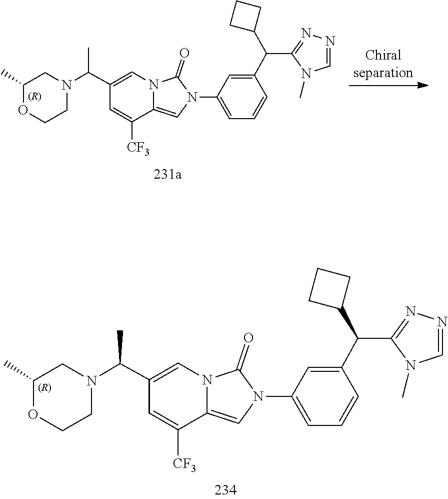

### Synthesis of 234

231a (830 mg) was purified by Chiral separation with the following conditions (Column: CHIRAL ART Cellulose-SB, 3*25 cm, 5 μm; Mobile Phase A: C02, Mobile Phase B: MeOH (0.1% 2M NH3-MeOH); Flow rate: 100 mL/min; Gradient: isocratic 20% B; Column Temperature (° C.): 35; Back Pressure (bar): 100; Wave Length: 220 nm; RT1 (min): 5.72; RT2 (min): 6.96; R T3 (min): 7.75; RT4 (min): 9.80; fourth peak is product) to afford 234 (117.6 mg, 13.89%) as a yellow solid.

LCMS-234 (ES, m/z): [M+H]⁺ 555. H-NMR-234 H NMR (400 MHz, DMSO, ppm): δ 1.02-1.08 (d, 3H), δ 1.21-1.31 (d, 3H), δ 1.72-1.81 (m, 6H), δ 2.03-2.12 (d, 2H), δ 2.31-2.49 (m, 1H), δ 2.58-2.63 (d, 1H), δ 274-2.83 (d, 1H), δ 3.17-3.25 (m, 1H), δ 3.39-3.43 (d, 4H), δ 3.43-3.51 (m, 1H), 3.68-3.82 (d, 1H), δ 4.24-4.28 (d, 1H), δ 7.05 (s, 1H), δ 7.18-7.24 (d, 1H), δ 7.31 (s, 1H), δ 7.41-7.48 (t, 1H), δ 7.62 (s, 1H), δ 7.66-7.70 (d, 1H), δ 7.73 (s, 1H), δ 8.33 (s, 1H).

### Example 235. Synthesis of Compound 235

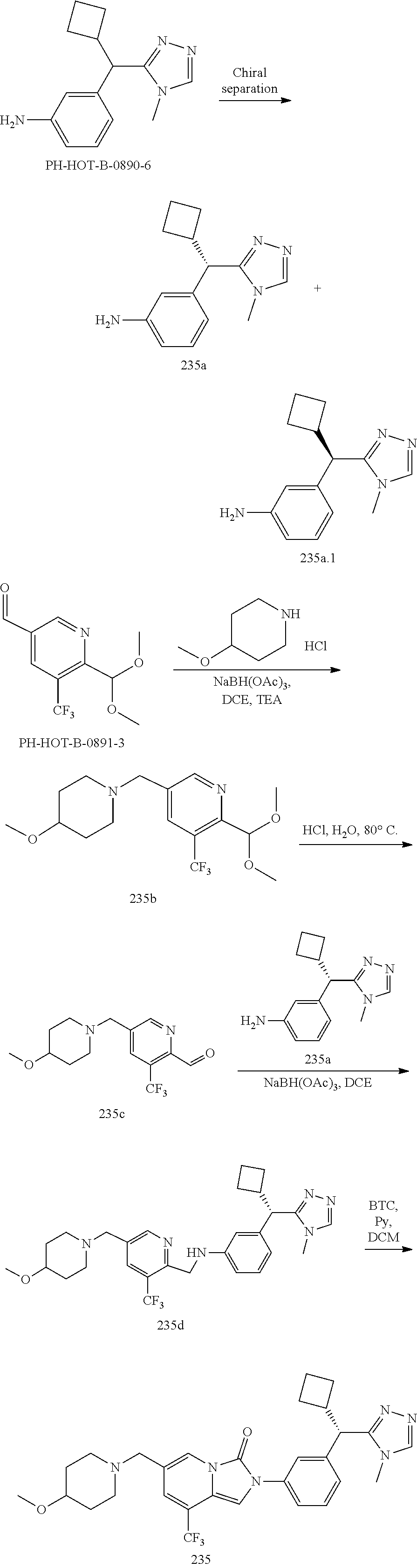

### 1. Synthesis of 235a

235a (900 mg) was purified by Chiral separation with the following conditions (Column: CHIRAL ART Cellulose-SB, 5*25 cm, 5 un; Mobile Phase A: C02, Mobile Phase B: EtOH-HPLC; Flow rate: 200 mL/min; Gradient: isocratic 30% B; Column Temperature (35° C.); Back Pressure (bar): 100; Wave Length: 220 nm; RT1 (min): 5.11; RT2 (min): 6.28; The first peak was 235a. The second peak was 235a.1. Sample Solvent: MeOH-HPLC; Injection Volume: 1 mL; Number of Runs: 30). This resulted in 235a (299.40 mg, 33.26%) as a yellow solid

### 2. Synthesis of 235b

To a stirred solution of 1-2 (2.5 g, 10.033 mmol, 1.00 equiv) and 4-methoxypiperidine hydrochloride (3.04 g, 20.066 mmol, 2 equiv) in DCE (30 mL) were added TEA. (2.03 g, 20,066 mmol, 2 equiv) at room temperature. The mixture was stirred for 30 min at room temperature. To the above mixture was added STAB (4.25 g, 20.066 mmol, 2 equiv) at room temperature. The resulting mixture was stirred for overnight at room temperature. The reaction was quenched by the addition of sat. NH4Cl (aq.) (50 ml) at room temperature. The resulting mixture was extracted with CH2Cl2 (3×80 mL). The resulting mixture was concentrated under reduced pressure. The residue was purified by silica gel column chromatography, eluted with PE/EA (20:1) to afford 235b (2.5 g, 67.95%) as a light yellow oil.

### 3, Synthesis of 235c

To a stirred solution of 235b (2 g, 5.741 mmol, 1 equiv) in HCl (1 M, 30 mL) at room temperature. The resulting mixture was stirred for overnight at 80° C. The residue was basified to pH 7 with sat. NH4HCO3 (aq.) (30 ml). The aqueous layer was extracted with EtOAc (3×50 mL). The resulting mixture was concentrated under vacuum. The residue was purified by silica gel column chromatography, eluted with PE/EA (20:1) to afford 235c (1.4 g, 76.63%) as a light yellow oil.

### 4. Synthesis of 235d

To a stirred solution of 235a (29940 mg, 0.990 mmol, 1.2 equiv) and 235c (200 mg, 0.825 mmol, 1 equiv) in DCE (3 mL) were added STAB (349.84 mg, 1.650 mmol, 2 equiv) and HOAc (49.56 mg, 0.825 mmol, 1 equiv) at room temperature. The resulting mixture was stirred for overnight at room temperature. The reaction was quenched by the addition of Sat. NH14Cl (aq.) (10 mL) at room temperature. The resulting mixture was extracted with CH2Cl2 (3×10 mL). The resulting mixture was concentrated under reduced pressure. The residue was purified by Prep-TLC (CH2Cl2/MeOH 20:1) to afford 235d (300 mg, 65.32%) as a yellow solid.

### 5, Synthesis of 235

To a stirred solution of 235d (200 mg, 0.378 mmol, 1 equiv) and pyridine (39.30 mg, 0.132 mmol, 0.35 equiv) in DCM (3 mL) were added Triphosgene (39.30 mg, 0.132 mmol, 0.35 equiv) at 0° C. The resulting mixture was stirred for 20 min at room temperature. The reaction was quenched by the addition of sat. NaHCO₃ (aq.) (5 mL) at room temperature. The resulting mixture was extracted with CH2Cl2 (3×10 mL). The resulting mixture was concentrated under reduced pressure. The residue was purified by reverse flash chromatography with the following conditions: C18 silica gel 120 g; mobile phase, MeCN in 0.1% NH4HCO3 water, 20% to 40% gradient in 10 min; detector, LV 254 nm. This resulted in 235 (85.9 mg, 28.18%) as a yellow solid.

LC-MS: (ES, m/z): [M+H]+: 555. H-NMR: (400 MHz, DMSO-d6, ppm): δ 1.32-1.50 (m, 2H), δ 1.62-1.85 (m, 7H), δ 2.03-2.21 (m, 3H), δ 2.63-2.73 (m, 2H), δ 3.18-3.25 (m, 5H), δ 3.31 (s, 2H), δ 3.45 (s, 3H), δ 4.25-4.27 (d, 1H), δ 7.01 (s, 1H), δ 7.18-7.20 (d, 1H), δ 7.31 (s, 1H), δ 7.44-7.48 (m, 1H), δ 7.60-7.62 (d, 2H), δ 7.70 (s, 1H), δ 8.33 (s, 1H).

### Example 236. Synthesis of Compound 236

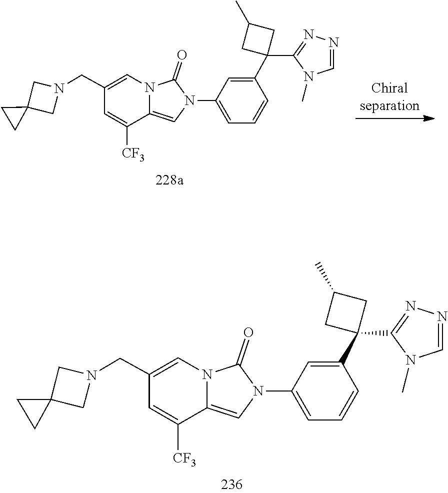 [SMILES: CC1CC(c2cccc(-n3cc4c(C(F)(F)F)cc(CN5CC6(CC6)C5)cn4c3=O)c2)(c2nncn2C)C1 ; Cn1cnnc1[C@]1(c2cccc(-n3cc4c(C(F)(F)F)cc(CN5CC6(CC6)C5)cn4c3=O)c2)C[C@H](C)C1]

### Synthesis of 236

228a (320 mg) was purified by Chiral separation with the following conditions (Column: CHIRALPAK IC, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 45% B to 45% B in 20 min; Wave Length: 220/254 nm; RT1 (min): 13.77; RT2 (min): 16.98; The second peak was the product. Sample Solvent: EtOH:DCM=1:1-HPLC; Injection Volume: 0.2 mL; Number of Runs: 15), This resulted in 236 (190.2 mg, 59.14%) as a yellow solid.

LC-MS: (ES, m/z): [M+H]+: 523. H-NMR: (400 MHz, DMSO-d6, ppm): δ 0.50 (s, 4H), δ 1.07-1.09 (s, 3H), δ 2.51-2.55 (m, 3H), δ 2.87-2.89 (d, 2H), δ 3.15 (s, 3H), δ 3.30-3.33 (m, 4H), δ 4.47 (s, 2H), δ 7.00 (s, 1H), δ 7.18-7.20 (d, 1H), δ 7.31 (s, 1H), δ 7.44-7.48 (m, 1H), δ 7.60-7.62 (d, 1H), δ 7.70 (s, 1H), δ 7.83 (s, 1H), δ 8.29 (s, 1H).

### Example 237. Synthesis of Compound 237

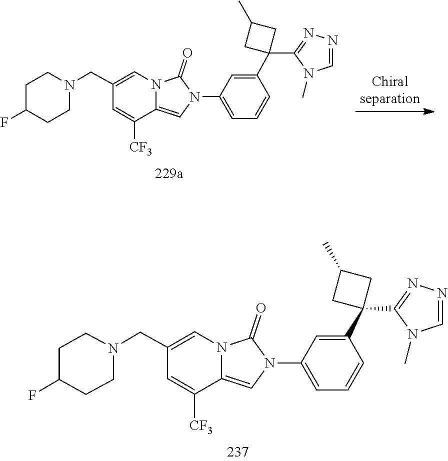 [SMILES: CC1CC(c2cccc(-n3cc4c(C(F)(F)F)cc(CN5CCC(F)CC5)cn4c3=O)c2)(c2nncn2C)C1 ; Cn1cnnc1[C@]1(c2cccc(-n3cc4c(C(F)(F)F)cc(CN5CCC(F)CC5)cn4c3=O)c2)C[C@H](C)C1]

### Synthesis of 237

229a (400 mg) was purified by Prep-Chiral-HPLC with the following conditions (Column: CHIRALPAK IC, 2*25 cm, 5 pnm; Mobile Phase A: Hex (0.5% 2M NH3-MeOH), Mobile Phase B: EtOH:DCM=1:1; Flow rate: 20 mL/in; Gradient: 55% B to 55% B in 16 min; Wave Length: 220/254 nm; RT1 (min): 10.58; RT2 (min): 14.29, the first peak is product) to afford 237 (176.0 mg, 43.56%) as a yellow solid.

LC-MS: (ES, m/z): [M+H]⁺ 543. H-NMR: (400 MHz, DMSO-d6, δ ppm): 1.03-1.13 (d, 3H), 1.61-1.88 (m, 2H), 1.88-1.96 (m, 2H), 2.36-2.46 (m, 2H), 2.51-2.67 (m, 3H), 2.83-2.95 (m, 2H), 3.21 (s, 3H), 3.32 (s, 2H), 4.64-4.77 (m, 1H), 7.01 (s, 1H), 7.24-7.26 (d, 1H), 7.38 (s, 1H), 7.44-7.52 (m, 1H), 7.63-7.66 (m, 1H), 7.69 (s, 1H), 7.83 (s, 1H), 8.29 (s, 1H).

### Example 238. Synthesis of Compound 238

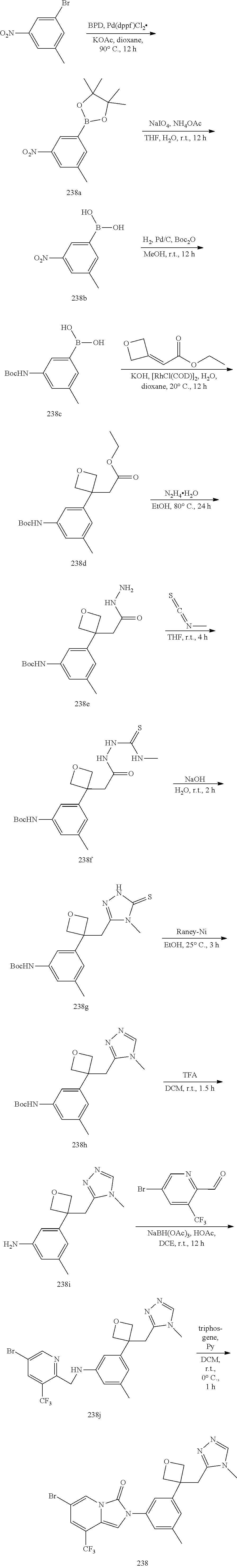

### 1. Synthesis of 238a

A mixture of 1-bromo-3-methyl-5-nitro-benzene (12 g, 55.55 mmol, 1 equiv), BPD (21.1 g, 83.3 mmol, L 5 equiv), KOAc (16.35 g, 166.64 mmol, 3 equiv) and Pd(dppf)Cl2 (3.25 g, 4.44 mmol, 0.08 equiv) in dioxane (150 mL) was degassed and purged with nitrogen atmosphere for 3 times, and then the mixture was stirred at 90° C. for 12 hr under nitrogen atmosphere. The reaction was poured to water (50 mL) and the resulting mixture was extracted with EtOAc (2×100 mL). The organic phase was washed with brine (50 mL), dried over anhydrous Na2SO4, concentrated in vacuum to give a residue. The residue was purified by silica gel column chromatography, eluted with PE/EA=5:1 to afford 238a (8 g, 55% yield) as a yellow oil.

### 2, Synthesis of 238b

To a solution of 238a (8 g, 30.4 mmol, 1 equiv) in THF (90 mL) and water (30 mL) was added NaIO4 (5.05 mL, 91.2 mmol, 3 equiv) and NH4OAc (11.7 g, 152 mmol, 5 equiv). The mixture was stirred at 25° C. for 12 hr. The reaction was added NaHSO4 and poured to water (100 mL) and the resulting mixture was extracted with EtOAc (2×100 mL). The organic phase was washed with brine (50 mL), dried over anhydrous Na2SO4, concentrated in vacuum to give a residue. The residue was purified by silica gel column chromatography, eluted with PE/EA=1:1 to afford 238b (7.2 g, 65.43% yield) as a white solid.

H-NMR: (400 MHz, DMSO-d6) δ 8.42 (d, J=8.0 Hz, 2H) 8.09 (s, 2H) 8.00-8.04 (m, 1H) 2.44 (s, 3H)

### 3. Synthesis of 238c

To a solution of 238b (2.3 g, 12.71 mmol, 1 equiv) in MeOH (40 mL) was added Pb/C (1.2 g, 10% purity) and Boc2O (14.6 mL, 63.5 mmol, 5 equiv). The suspension was degassed and purged with hydrogen for 3 times. The mixture was stirred under hydrogen (15 Psi) at 25° C. for 12 h. The reaction mixture was diluted with water (40 mL) and extracted with EtOAc (3×40 ml). The combined organic layers were dried over anhydrous Na2SO4, filtered and concentrated under reduced pressure to give a residue. The residue was purified by silica gel column chromatography, eluted with CH2Cl2/MeOH=1:1 to afford 238e (7.9 g, 82.5%) as a white solid. H-NMR: (400 MHz, DMSO-d6) δ 9.12 (s, 1 H), 8.45 (s, 1H), 7.86 (s, 2H), 7.54 (s, 1H), 7.28 (s, 1K), 2.22 (s, 3H), 1.44 (s, 9H).

### 4. Synthesis of 238d

To a stirred solution of chlororhodium;(1Z,5Z)-cycloocta-1,5-diene (102 mg, 208 umol, 0.023 equiv) in dioxane (45 mL) was added KOH (558 mg, 9.96 mmol, 1.1 equiv) in water (35 mL) dropwise at 20° C. under nitrogen atmosphere. The resulting mixture was stirred for 0.5 h at 25° C. under nitrogen atmosphere. To the above mixture were added 238c (3.5 g, 13.9 mmol, 1.54 equiv) and ethyl 2-(oxetan-3-ylidene)acetate (1.29 g, 9.05 mmol, 1 equiv) at 25° C. for 17.5 hr under nitrogen atmosphere. The reaction mixture was diluted with water (80 mL) and extracted with EtOAc (3×80 mL). The combined organic layers were dried over anhydrous Na2SO4, filtered and concentrated under reduced pressure to give a residue. The residue was purified by silica gel column chromatography, eluted with EtOAc/PE=1:1 to afford 238d (3.66 g, 57.8%) as a brown solid.

H-NMR: (400 MHz, CHLOROFORM-d) δ 7.09 (s, 1H), 6.92 (s, 1H), 6.61 (s, 1H), 6.42 (s, 1H), 4.98 (d, J=6.0 Hz, 2H), 4.82 (d, J=6.0 Hz, 2H), 4.05-3.98 (m, 2H), 3.07 (s, 2H), 2.29 (s, 3), 2.04 (s, 1H), 1.50 (s, 9H), 1.13 (t, J=7.2 Hz, 3H).

### 5. Synthesis of 238e

To a solution of 238d (3.66 g, 104 mmol, 1 equiv) in EtOH (30 mL) was added N2H4·H2O (8.31 mL, 167, mmol, 98% purity, 16 equiv). The mixture was stirred at 80° C. for 12 hr. The reaction mixture was concentrate in vacuum to remove EtOH (20 mL), then the mixture was diluted with water (30 mL), and extracted with CH2Cl2 (3×30 mL). The combined organic layers were dried over anhydrous Na2SO4, filtered and concentrated under reduced pressure to afford 238e (3.05g, crude) as a yellow solid.

### 6. Synthesis of 238f

To a solution of 238e (3.05 g, 9.09 mmol, 1 equiv) in THE (40 mL) was added methylimino(thioxo)methane (1.33 g, 18.19 mmol, 1.24 mL, 2 equiv). The mixture was stirred at 25° C. C for 4 hr. The reaction mixture was diluted with water 40 ml, and extracted with EtOAc (3×40 mL). The combined organic layers were dried over anhydrous Na2SO4, filtered and concentrated under reduced pressure to afford 238f (4.71 g, crude) as a white solid.

### 7. Synthesis of 238g

To a solution of NaOH (3.69 g, 92.24 mmol, 8 equiv) in water (40 mL) was added 238f (71 g, 11.53 mmol, 1 equiv).The mixture was stirred at 25° C. for 2 hr. The reaction mixture was diluted with water (40 mL), adjusted pH to 5 by 1N HC, filtered and the filter cake was concentrated in vacuum to afford 238g (4.07 g, 90.4%) as a white solid.

H-NMR-238g: (400 MHz, DMSO-d6): δ 13.49 (s, 1H), 9.39-9.04 (i, 1H), 7.16 (s, 1H), 6.99 (s, 1H), 6.44 (s, 1H), 4.80-4.77 (m, 2H) 4.77-4.74 (i, 2H), 3.43 (s, 2H), 2.84 (s, 31), 2.19 (s, 3H), 1.46 (s, 9H)

### 8. Synthesis of 238h

To a solution of 238g (4.07 g, 10.4 mmol, 1 equiv) in EtOH (50 mL) was added Raney-Ni (8 g, 93.38 mmol, 8.96 equiv).The mixture was stirred at 25° C. for 3 hr under nitrogen atmosphere. The reaction mixture was filtered and the filtrate was concentrated under reduced pressure to afford 238h (1.68 g, crude) as a white solid.

### 9. Synthesis of 238i

To a solution of 238h (1-68 g, 4.69 mmol, 1 equiv) in DCM (16 m-L) was added TEA (8 mL). The mixture was stirred at 25° C. for 1.5 hr. The reaction mixture was concentrated under reduced pressure to afford 238i (1.2 g, crude) was obtained as a purple oil.

### 10. Synthesis of 238j

To a solution of 238i (580 mg, 2.25 mmol, 1 equiv) and 5-bromo-3-(trifluoromethyl)picolinaldehyde (456 mg, 1.80 mmol, 0.8 equiv) in MeOH (30 mL) was added HOAc (385 uL, 6.74 mmol, 3 equiv). The mixture was stirred at 25° C. for 1 h under nitrogen atmosphere. Then NaBH3CN (282 mg 4.49 mmol, 2 equiv) was added, the result mixture was stirred at 25° C. for 1 h under nitrogen atmosphere. The mixture was concentrated under reduced pressure and diluted with NaHCO₃ (100 ml), extracted with DCM (30 ml×3), dried over anhydrous Na2SO4, filtered and the filtrate was concentrated under reduced pressure to give a residue. The residue was purified by silica gel column chromatography, eluted with CH2Cl2/MeOH=10:1 to afford 238j (440 mg, 39.4%) as a yellow solid.

### 11. Synthesis of 238

To a solution of 238j (220 mg, 443.26 umol, 1 equiv) in DCM (10 mL) was added pyridine (214 uL, 2.66 mmol, 6 equiv) and bis(trichloromethyl) carbonate (52.6 mg, 177 umol, 0.4 equiv) at 0° C. The mixture was stirred at 0° C. for 1 h under nitrogen atmosphere. Then pyridine (214 uL, 2.66 m mol, 6 equiv) and bis(trichloromethyl) carbonate (52.61 mg, 177.30 umol, 0.4 equiv) was added. The mixture was stirred at 0° C. for 1 h under nitrogen atmosphere. The reaction was quenched with Sat.NaHCO3 (20 mL). The aqueous layer was extracted with CH2Cl2 (3×20 mL). The combined organic layers were dried over anhydrous Na2SO4, filtered and the filtrate was concentrated under reduced pressure to give a residue. The residue was purified by silica gel column chromatography, eluted with CH2Cl2/MeOH=1:1 to give crude product. The crude product (130 mg) was purified by Prep-HPLC with the following conditions (Column: Phenomenex Luna C18 150*30 mm*5 um; Mobile Phase A: Water (0.2% formic acid), Mobile Phase B: ACN; Flow rate: 50 mL/min; Gradient: 15% B to 55% B in 8 min; Wave Length: 220 nm; RT1 (min): 9.0) to afford 238 (52.5 mg, 10.53%) as a yellow solid,

LCMS: (ES, m/z): [M+H]⁺ 522.0/524.0. H-NMR: (400 MHz, DMSO-d6) δ 8.22 (s, 1H), 8.02 (s, 1H), 7.56 (s, 1H), 7.42 (s, 1H), 7.30-7.11 (m, 2H), 6.72 (s, 1H), 4.98-4.87 (m, 4H), 3.52 (s, 2H), 2.99 (s, 3H), 2.30 (s, 3H).

### Example 239. Synthesis of Compound 239

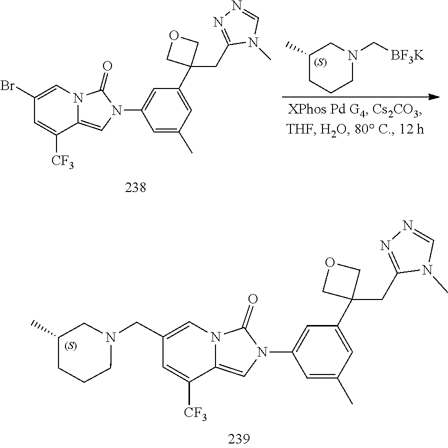

### Synthesis of 239

To a solution of 238 (80 mg, 153 umol, 1 equiv) and BF₃ salt (67.1 mg, 306 umol, 2 equiv) in THF (4 mL) and water (1 mL) was added Cs2CO3 (149.71 mg, 459.49 umol, 3 eq) and dicyclohexyl-[2-(2,4,6-triisopropylphenyl)phenyl]phosphane; methanesulfonate;[2-[2-(methylamino) phenyl]phenyl]palladium(1+) (13.1 mg, 15.3 umol, 0.1 eq). The mixture was stirred at 80° C. for 12 h under nitrogen atmosphere. The mixture was diluted with water (50 ml) and extracted with DCM (20 ml×3). The combined organic was dried over anhydrous Na2SO4, filtered and the filtrate was concentrated under reduced pressure to give a residue. The residue was purified by Prep-HPLC with the following conditions (Column: Phenomenex Luna C18 150*30 mm*5 um; Mobile Phase A: Water (0.2% formic acid), Mobile Phase B: ACN; Flow rate: 35 mL/min; Gradient: 15% B to 55% B in 8 min; Wave Length: 220 nm; RT (min): 10.0) to afford 239 (10.8 mg, 12.7%) as a yellow solid.

LCMS: (ES, m/z): [M+H]⁺ 555.3. H-NMR: (400 MHz, METHANOL-d4) δ 8.38 (s, 1H), 8.22 (s, 1H), 7.85 (s, 1H), 7.47 (s, 1H), 7.14 (d, J=7.6 Hz, 2H), 7.06 (s, 1H), 6.75 (s, 1H), 5.07 (s, 4H), 3.79 (s, 2H), 3.66 (s, 2H), 3.22-3.16 (m, 1H), 2.98 (s, 3H), 2.54-2.44 (m, 1H), 2.38 (s, 3H), 2.25-2.17 (m, 1H), 1.93-1.65 (m, 5H), 1.16-1.05 (m, 1H), 0.98 (d, J=6.4 Hz, 3H)

### Example 240. Synthesis of Compound 240

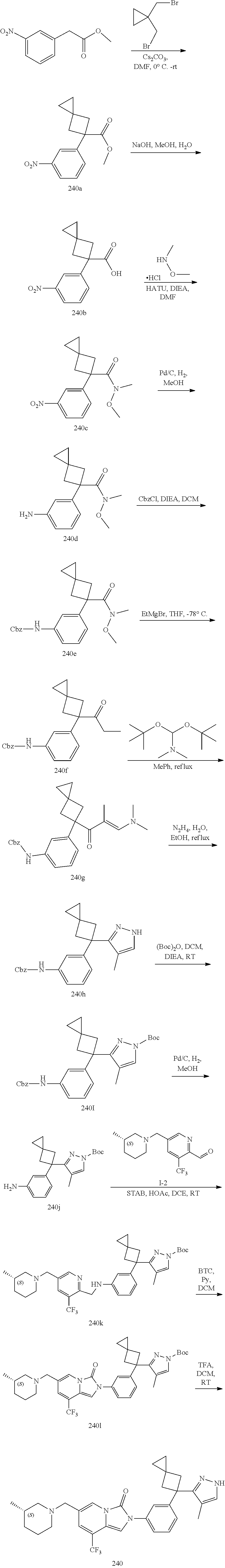

### 1. Synthesis of 240a

To a stirred solution of methyl 2-(3-nitrophenyl)acetate (50 g, 256.182 mmol, 1 equiv) in DMF (800 mL) was added Cs2CO3 (417.35 g, 1280,910 mmol, 5 equiv) at 0° C. under nitrogen atmosphere. The resulting mixture was stirred for 2h at 0° C. under nitrogen atmosphere. To the above mixture was added 1,1-bis(bromomethyl)cyclopropane (175.17 g, 768.546 mmol, 3 equiv) at 0 degrees C. The resulting mixture was stirred for additional overnight at room temperature. The reaction was quenched with saturated NH4Cl(aq.) (3 L) at room temperature. The aqueous layer was extracted with EtOAc (2×800 mL). The resulting mixture was concentrated under reduced pressure. The residue was purified by silica gel column chromatography, eluted with PE/EtOAc (50:1) to afford 240a (37 g, 50.86%) as a colorless oil.

### 2. Synthesis of 240b

Into a 1000 mL 3-necked round-bottom flask was added 240a (37 g, 141.612 mmol, 1 equiv) and NaOH (5.66 g, 141.612 mmol, 1 equiv), MeOH (370 mL), H2O (20 mL) at room temperature. The resulting mixture was stirred overnight at room temperature. The mixture was acidified to pH 5 with HCl (4M). The resulting mixture was extracted with EtOAc (3×500 mL). The resulting mixture was concentrated under vacuum. The residue was purified by silica gel column chromatography, eluted with PE/EA (10:1) to afford 240b (34 g, 97.11%) as a yellow oil.

### 3. Synthesis of 240c

Into a 500 mL, 3-necked round-bottom flask were added 240b (17 g, 68.8 mmol, 1 equiv) and DMF (170 mL), HATU (39.21 g, 103.2 mmol, 1.5 equiv), DIEA (26.42 g, 206.4 mmol, 3 equiv), N,O-dimethylhydroxylamine hydrochloride (10.11 g, 103.2 mmol, 1.5 equiv) at room temperature. The resulting mixture was stirred overnight at room temperature. The resulting mixture was diluted with water (800 mL). The aqueous layer was extracted with EtOAc (3×200 mL). The resulting mixture was concentrated under vacuum. The residue was purified by silica gel column chromatography, eluted with PE/EA (10:1) to afford 240c (17 g, 40.32%) as a yellow oil.

### 4. Synthesis of 240d

To a solution of 240c (18.7 g, 64.412 mmol, 1 equiv) in MeOH (400 mL) was added Pd/C (1.87 g, 10%) under nitrogen atmosphere in a 1 L round-bottom flask. The mixture was hydrogenated at room temperature for overnight under hydrogen atmosphere using a hydrogen balloon, filtered through a Celite pad and concentrated under reduced pressure. This resulted in 240d (16 g, 95.42%) as a yellow oil.

### 5. Synthesis of 240e

To a stirred solution of 240d (16 g, 61,459 mmol, 1 equiv) and DIEA (27.80 g, 215.107 mmol, 3.5 equiv) in DCM (500 mL) were added CbzCl (20.97 g, 122.918 mmol, 2 equiv) at room temperature under nitrogen atmosphere. The resulting mixture was stirred overnight at room temperature. The residue was washed with water (500 mL). The aqueous layer was extracted with DCM (200 mL). The combined organic layers were dried over anhydrous Na2SO4, After filtration, the filtrate was concentrated under reduced pressure. The residue was purified by silica gel column chromatography, eluted with PE/EA (5:1) to afford 240e (22 g, 90.75%) as a yellow oil.

### 6. Synthesis of 240f

To a stirred solution of 240e (17.2 g, 43.603 mmol, 1 equiv) in TH-F (200 mL) was added EtMgBr (109 mL, 109.00 mmol, 2.5 equiv) dropwise at −78° C. under nitrogen atmosphere. The resulting mixture was stirred for 3h at room temperature under nitrogen atmosphere. The reaction was quenched with saturated NH4Cl (aq.) (500 mL) at room temperature. The aqueous layer was extracted with EtOAc (2×500 mL). The organic layers were concentrated under reduced pressure. The residue was purified by silica gel column chromatography, eluted with PE/EA (10:1) to afford 240f (7.13 g, 44.99%) as a yellow oil.

### 7. Synthesis of 240g

To a stirred solution of 240f (11.83 g, 32.549 mmol, 1 equiv) in Toluene (150 mL) was added [bis(tert-butoxy)methyl]dimethylamine (33.09 g, 162.745 mmol, 5 equiv) at room temperature under nitrogen atmosphere. The resulting mixture was stirred overnight at 110° C. The resulting mixture was concentrated under vacuum. This resulted in 240g (13 g, crude) as a yellow oil.

### 8. Synthesis of 240h

To a stirred solution of 240g (13 g, 31.10 mmol, 1 equiv) in EtOH (200 mL) was added hydrazine hydrate (98%)(15.55 g, 311.00 mmol, 10 equiv) at room temperature. The resulting mixture was stirred for 6h at 80° C. The reaction was quenched with water (600 mL) at room temperature. The aqueous layer was extracted with EtOAc (2×500 mL). The resulting mixture was concentrated under vacuum. The residue was purified by silica gel column chromatography, eluted with PE/EA (2:1) to afford 240h (6 g, 49.87%) as a light yellow solid.

### 9. Synthesis of 240i

To a stirred solution of 240h (3 g, 7.742 mmol, 1 equiv) and (Boc)₂₀ (3.38 g, 15.484 mmol, 2 equiv) in DCM (50 mL) were added DIEA (3.00 g, 23.226 mmol, 3 equiv) at room temperature under nitrogen atmosphere. The resulting mixture was stirred overnight at room temperature. The reaction was quenched with water (100 mL) at room temperature. The aqueous layer was extracted with DCM (2×100 mL). The organic layers were concentrated under reduced pressure. The residue was purified by silica gel column chromatography, eluted with PE/EA (10:1) to afford 240i (2.7 g, 67.94%) as a white solid.

### 10, Synthesis of 240j

To a solution of 240i (2.7 g, 5.537 mmol, 1 equiv) in MeOH (50 mL) was added Pd/C (270 mg, 10%) under nitrogen atmosphere in a 100 mL round-bottom flask. The mixture was hydrogenated at room temperature for 3h under hydrogen atmosphere using a hydrogen balloon, filtered through a Celite pad and concentrated under reduced pressure. This resulted in 240j (1.7 g, 78.17%) as an off-white solid. The crude product was used in the next step directly without further purification.

### 11. Synthesis of 240k

To a stirred solution of 240j (300 mg, 0.849 mmol, 1 equiv) and 1-2 (267.29 mg, 0.934 mmol, 1.1 equiv) in DCE (5 mL) were added HOAc (5.10 mg, 0.085 mmol, 0.1 equiv) and STAB (359.76 mg, 1.698 mmol, 2 equiv) at room temperature under nitrogen atmosphere. The resulting mixture was stirred overnight at room temperature. The reaction was quenched with saturated NaHCO₃ (aq.) (50 mL) at room temperature. The aqueous layer was extracted with DCM (2×30 mL). The organic layers were concentrated under reduced pressure. The residue was purified by Prep-TLC (DCM/MeOH 30:1) to afford 240k (400 mg, 68.76%) as an off-white solid.

### 12. Synthesis of 2401

To a stirred solution of 240k (400 mg, 0.641 mmol, 1 equiv) and Pyridine (507.24 mg, 6.410 mmol, 10 equiv) in DCM (10 mL) were added Triphosgene (76.11 mg, 0.256 mmol, 0.4 equiv) at room temperature under nitrogen atmosphere. The resulting mixture was stirred for 10 min at room temperature. The reaction was quenched with saturated NaHCO3 (aq.) (100 mL) at room temperature. The aqueous layer was extracted with DCM (2×30 mL). The resulting mixture was concentrated under vacuum. This resulted in 2401 (200 mg, 43.20%) as a yellow solid. The crude product was used in the next step directly without further purification.

### 13. Synthesis of 240

To a stirred solution of 2401 (200 mg) in DCM (5 mL) was added TEA (2 mL) at room temperature. The resulting mixture was stirred for 1h at room temperature. The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 60% B to 90% B in 8 min, Wave Length: 220 nm; RT1 (min): 7.53) to afford 240 (106.2 mg, 61.08%) as a yellow solid.

LC-MS: (ES, m/z): [M+H]⁺ 550. H-NMR: (400 MHz, DMSO-d6, δ ppm): 0.40-0.50 (m, 2H), 0.50-0.60 (m, 2H), 0.79-0.95 (m, 4H), 1.43-1.53 (m, 1H), 1.53-1.73 (m, 4H), 1.74 (s, 3H), 1.86-1.92 (m, 1H), 2.67-2.81 (m, 4H), 3.02-3.12 (m, 2H), 3.24 (s, 2H), 7.00 (s, 1H), 7.23-7.39 (m, 3H), 7.39-7.45 (m, 1H), 7.53-7.55 (m, 1H), 7.66 (s, 1H), 7.84 (s, 1H), 12.26 (s, 1H).

### Example 241. Synthesis of Compound 241

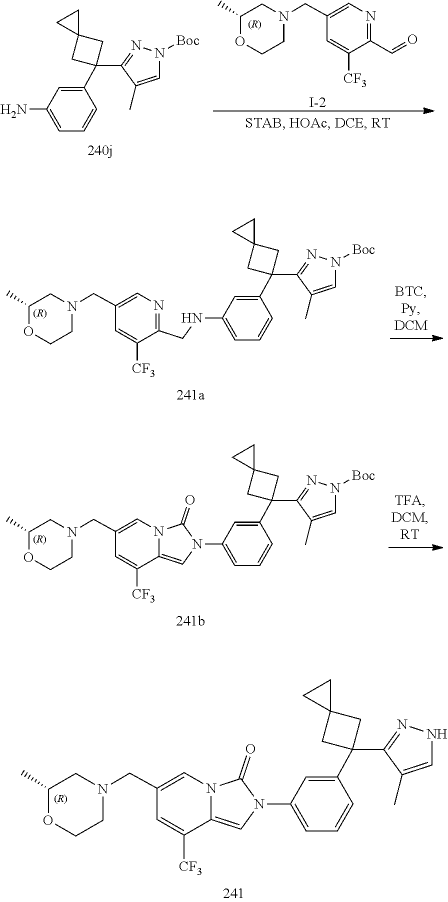

### 1. Synthesis of 241a

To a stirred solution of 240j (300 mg, 0.849 mmol, 1 equiv) and I-2 (269.13 mg, 0.934 mmol, 11 equiv) in DCE (5 mL) was added HOAc (510 mg, 0.085 mmol, 0.10 equiv) and STAB (359.76 mg, 1,698 mmol, 2 equiv) at room temperature under nitrogen atmosphere. The resulting mixture was stirred for overnight at room temperature. The reaction was quenched with saturated NaHCO3 (aq.) (100 mL) at room temperature. The aqueous layer was extracted with DCM (2×50 mL). The organic layers were concentrated under reduced pressure. The residue was purified by Prep-TLC (DCM/MeOH 30:1) to afford 241a (400 mg, 71.55%) as a white solid,

### 2. Synthesis of 241b

To a stirred solution of 241a (400 mg 0.639 mmol, 1 equiv) and Pyridine (505.64 mg, 6,390 mmol, 10 equiv) in DCM (10 mL) was added Triphosgene (75.87 mg, 0,256 mmol, 0.4 equiv) at room temperature under nitrogen atmosphere. The resulting mixture was stirred for 10 min at room temperature. The reaction was quenched with saturated NaHCO₃ (aq.) (100 mL) at room temperature. The aqueous layer was extracted with DCM (2×30 ml). The resulting mixture was concentrated under vacuum. This resulted in 241b (300 mg, 64.81%) as a yellow solid. The crude product was used in the next step directly without further purification.

### 3. Synthesis of 241

To a stirred solution of 241b (300 mg) in DCM (8 mL) was added TFA (2 mL) at room temperature. The resulting mixture was stirred for 1h at room temperature. The resulting mixture was concentrated under vacuum. The crude product was purified by Prep-HPLC with the following conditions (Column: XBridge Prep OBD CIS Column, 30*150 mm, 5 μm; Mobile Phase A: Water (10 mmol/L N141C03), Mobile Phase B: ACN; Flow rate: 60 mL/min; Gradient: 45% B to 75% B in 8 min, Wave Length: 220 inn; RT1 (min): 7.52) to afford 241 (151.3 mg, 58.93%) as a yellow solid.

LC-MS: (ES, m/z): [M+H]⁺ 552. H-NMR: (400 MHz, DMSO-d6, δ ppm): 0.41-0.48 (m, 2H), 0.48-0.52 (m, 2H), 1.03-1.05 (d, 3H), 1.71-1.76 (m, 4H), 2.02-2.08 (m, 1H), 2.65-2.75 (m, 4H), 3.02-3.05 (m, 2H), 3.23-3.28 (m, 2H), 3.35 (s, 2H), 3.46-3.51 (m, 2H), 3.73-3.75 (m, 2H), 7.01 (s, 1H), 7.23-7.27 (m, 2H), 7.27-7.45 (m, 2H), 7.52-7.54 (m, 1H), 7.70 (s, 1H), 7.83 (s, 1H), 12.21 (s, 1H).

### Example 242. Synthesis of Compound 242

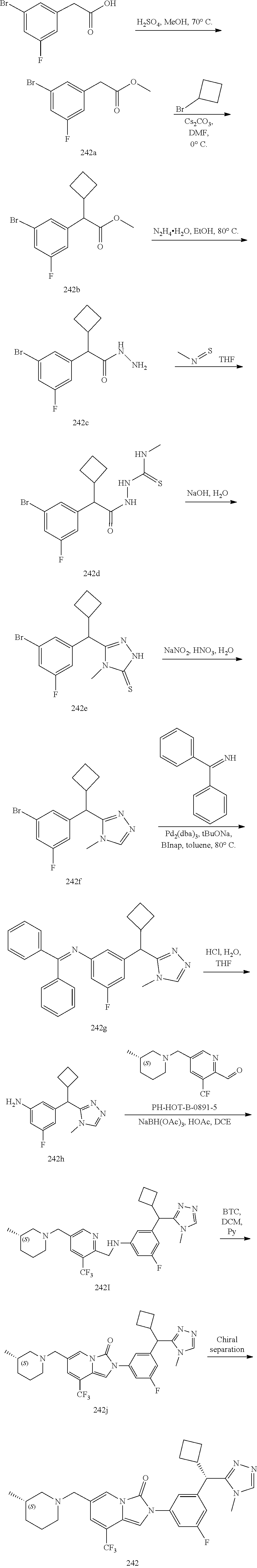

### 1. Synthesis of 242a

A solution of (3-bromo-5-fluorophenyl)acetic acid (10 g, 4912 mol, 1 equiv) and H2SO4 (0.42 g, 4.291 mmol, 0.1 equiv) in MeOH (100 mL) was stirred for 3h at 70° C. under nitrogen atmosphere. The reaction was quenched with water/ice (200 mL) at room temperature. The aqueous layer was extracted with EtOAc (3×100 mL).The resulting mixture was concentrated under reduced pressure. This resulted in 242a (10 g, 94.32%) as a colorless oil.

### 2. Synthesis of 2426

Into a 500 mL 3-necked round-bottom flask were added 242a (10 g, 40.476 mmol, 1 equiv), Cs2CO3 (39.56 g, 121.428 mmol, 3 equiv) and DMF (100 mL) at 0′CC. The resulting mixture was stirred for 2h at room temperature under nitrogen atmosphere. To the above mixture was added bromocyclobutane (10.93 g, 80,952 mmol, 2 equiv) at room temperature. The resulting mixture was stirred overnight at room temperature. The reaction was quenched by saturated NH4Cl (aq.) (300 mL) at room temperature. The aqueous layer was extracted with EtOAc (1×100 mL). The resulting mixture was concentrated under vacuum. The residue was purified by silica gel column chromatography, eluted with PE/EA (5:1) to afford 242b (10 g, 74.66%) as a yellow oil, 3. Synthesis of 242c

Into a 500 mL 3-necked round-bottom flask were added 242b (10 g, 33,205 mmol, 1 equiv), N2H4·H2O (16.62 g, 332.05 mmol, 10 equiv) and EtOH (100 mL, 2754,023 mmol) at room temperature. The resulting mixture was stirred overnight at 80′C. The mixture was allowed to cool down to room temperature. The reaction was quenched by water (100 mL) at room temperature. The aqueous layer was extracted with DCM (3×100 mL). The resulting mixture was concentrated under vacuum. This resulted in 242c (10 g, 91.00%) as a yellow oil.

### 4. Synthesis of 242d

Into a 500 ml, 3-necked round-bottom flask were added 242c (10 g, 33.205 mmol, 1 equiv), methyl isothiocyanate (5.34 g, 73.051 mmol, 2.2 equiv) and tetrahydrofuran (150 mL) at room temperature. The resulting mixture was stirred overnight at room temperature. The resulting mixture was diluted with water (100 ml) and moved the THF under vacuum. The precipitated solids were collected by filtration and washed with water (20 mL). This resulted in 242d (10 g, 74.83%) as a white solid.

### 5. Synthesis of 242e

To a stirred mixture of NaOH (4.27 g, 106,876 mmol, 4 equiv) in H2O (100 ml) was added 242d (10 g, 26,719 mmol, 1 equiv) at room temperature under nitrogen atmosphere. The resulting mixture was stirred for 2h at room temperature. The mixture was acidified to pH 5 with HC (aq.). The aqueous layer was extracted with DCM (2×100 mL). The resulting mixture was concentrated under vacuum. This resulted in 242e (9 g, 86.04%) as a yellow oil.

### 6, Synthesis of 242f

To a stirred mixture of 242e (10 g, 28,069 mmol, 1 equiv) and NaNO2 (19.37 g, 280.690 mmol, 10 equiv) in H2 O (90 mL) was added N03 (280 ml, 280.690 mmol, 10 equiv, 1M) at 0° C. The resulting mixture was stirred for 2h at room temperature. The mixture was neutralized to pH 8 with NaHCO₃ (aq.). The aqueous layer was extracted with DCM (1×90 mL).The resulting mixture was concentrated under vacuum. The residue was purified by silica gel column chromatography, eluted with DCM/MeOH (500:1) to afford 242f (3 g, 30.33%) as an off-white solid.

### 7. Synthesis of 242g

Into a 100 mL 3-necked round-bottom flask were added 242f (3 g, 9.254 mmol, 1 equiv), benzenemethanimine (1.68 g, 9.254 mmol, 1 equiv), Pd2(dba)3·CHCl3 (0.48 g, 0.463 mmol, 0.05 equiv), Binap (0.43 g, 0,694 mmol, 0.075 equiv), t-BuONa (1.07 g, 1, 105 mmol, 1.2 equiv) and toluene (30 mL) at room temperature. The resulting mixture was stirred overnight at 80° C. under nitrogen atmosphere. The mixture was allowed to cool down to room temperature. The reaction was quenched by saturated NH4Cl (aq.) (200 mL) at room temperature. The aqueous layer was extracted with EtOAc (2×100 mL). The resulting mixture was concentrated under vacuum. This resulted in 242g (4 g, 61.10%) as a yellow oil. The crude product was used in the next step directly without further purification,

### 8. Synthesis of 242h

Into a 100 mL 3-necked round-bottom flask was added 242g (2.5 g, 5.889 mmol, 1 equiv), HCl (1M)(20 mL) and THE (5 mL) at room temperature. The resulting mixture was stirred overnight at room temperature. The mixture was basified to pH 7 with saturated NaHCO₃ (aq.). The resulting mixture was concentrated under vacuum. The crude product was purified by reverse flash chromatography with the following conditions: column, C18; mobile phase, A: water (10 mmol/L 0.1% NH4HCO3), B: MeCN, 15% B to 30% B gradient in 20 min; detector, UV 254 am. The residue was dried by lyophilization to afford 242h (300 mg, 18.00%) as a white solid.

### 9. Synthesis of 242i

To a stirred solution of 242h (280 mg, 1.076 mmol, 1 equiv) and 1-2 (677.48 mg, 2.368 mmol, 2.2 equiv) in DCE (5 mL) were added HOAc (64.59 mg, 1.076 mmol, 1.00 equiv) and NaBH(OAc)3 (455.93 mg 2.152 mmol, 2 equiv) at room temperature. The resulting mixture was stirred for 6h at room temperature. The reaction was quenched with saturated NaHCO₃ (aq.) (100 mL) at room temperature. The aqueous layer was extracted with DCM (2×20 mL). The organic layers were concentrated under reduced pressure. The residue was purified by Prep-TLC (DCM/MeOH 15:1) to afford 242i (320 mg, 52.14%) as a light yellow oil,

### 10. Synthesis of 242j

To a stirred solution of 242i (300 mg, 0.565 mmol, 1 equiv) and Pyridine (447.22 mg, 5.654 mmol, 10.00 equiv) in DCM (10 ml) were added Triphosgene (83.89 mg, 0.283 mmol, 0.50 equiv) at room temperature. The resulting mixture was stirred for 10 min at room temperature. The reaction was quenched with saturated NaHCO₃ (aq.) (100 mL) at room temperature. The aqueous layer was extracted with DCM (2×20 mL). The organic layers were concentrated under reduced pressure. The residue was purified by Prep-TLC (DCM/MeOH 15:1) to afford 242j (200 mg, 60.38%) as a yellow solid.

### 11. Synthesis of 240

242j (200 mg) was purified by Prep-Chiral-HPLC with the following conditions (Column: CHIRALPAK IC, 2*25 cm, 5 μm; Mobile Phase A: Hex (0.5% 2M NH3-1MeOH), Mobile Phase B: EtOH:DCM=1:1; Flow rate: 20 mL/min; Gradient: 40% B to 40% B in 13 min; Wave Length: 220/254 nm; RT1 (min): 9.72; RT2 (min): 11.1, the first peak is product) to afford 242 (65.7 mg, 32.52%) as a yellow solid.

LC-MS: (ES, m/z): [M+H]⁺ 557. H-NMR: (400 MHz, DMSO-d6, δ ppm): 0.82-0.93 (m, 4H), 1.41-1.55 (m, 1H), 1.58-1.78 (m, 5H), 1.78-1.93 (m, 5H), 2.06-2.08 (m, 1H), 2.65-2.81 (m, 2H), 3.22-3.31 (m, 3H), 3.47 (s, 3H), 4.30-4.33 (d, 1H), 7.03-7.06 (m, 2H), 7.42 (s, 1H), 7.65 (s, 1H), 7.69-7.71 (m, 2H), 8.36 (s, 1H).

### Example 243. Synthesis of Compound 243

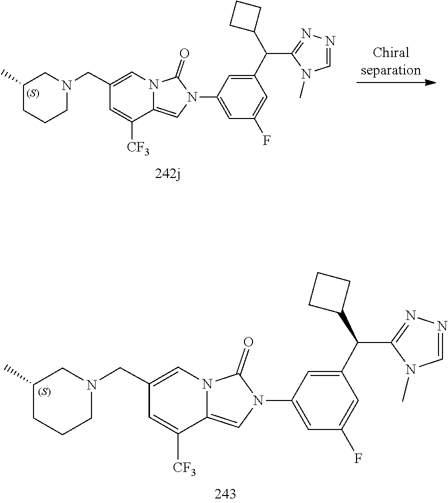

### Synthesis of 243

242j (200 mg) was purified by Prep-Chiral-HPLC with the following conditions (Column: CHIRALPAK IC, 2*25 cm, 5 pnm; Mobile Phase A: Hex (0.5% 2M NH₃-MeOH), Mobile Phase B: EtOH:DCM=1:1; Flow rate: 20 mL/min; Gradient: 40% B to 40% B in 13 min; Wave Length: 220/254 nm; RT1 (min): 9.72; RT2 (min): 11.1, the second peak is product) to afford 243 (74.3 mg, 36.78%) as a yellow solid,

LC-MS-243 (ES, m/z): [M+H]⁺ 557. H-NMR-243 (400 MHz, DMSO-d6, δ ppm): 0.82-0.93 (m, 4H), 1.41-1.55 (m, 1H), 1.58-1.78 (m, 5H), 1.78-1.93 (m, 5H), 2.06-2.08 (m, 1H), 2.65-2.81 (m, 2H), 3.22-3.31 (m, 3H), 3.47 (s, 3H), 4.30-4.33 (d, 1H), 7.03-7.06 (m, 2H), 7.42 (s, 1H), 7.65 (s, 1H), 7.69-7.71 (m, 2H), 8.36 (s, 1H).

### Example 244. Synthesis of Compound 244

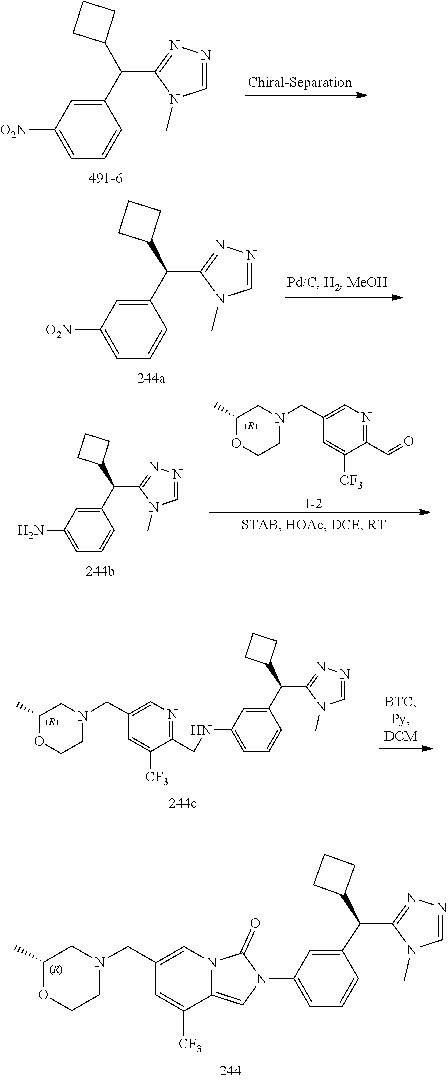

### 1. Synthesis of 244a

491-6 (40 g) was separated by Prep-SFC with the following conditions (Column: CHIRAL ART Cellulose-SC, 5*25 cm, 10 μm; Mobile Phase A: C02, Mobile Phase B: ACN: MeOH=1:1 (1% 2M NH3-MeOH); Flow rate: 250 mL/min; Gradient: isocratic 36% B; Column Temperature (° C.): 35; Back Pressure (bar): 100; Wave Length: 220 nm; RT1 (min): 9.7; RT2 (min): 12; The first peak was product) to afford 244a (13 g, 32.50%) as a off-white solid.

### 2. Synthesis of 244b

To a solution of 244a (22 g, 76:751 mmol, 1 equiv, 95%) in 250 ml, MeOH was added Pd/C (20%, 5 g) under nitrogen atmosphere. The mixture was hydrogenated at room temperature for overnight under hydrogen atmosphere using a hydrogen balloon, filtered through a Celite pad and concentrated under reduced pressure. This resulted in 244b (18 g, 92.91%) as a white solid.

### 3. Synthesis of 244c

To a stirred mixture of 244b (200 mg, 0.825 mmol, 1 equiv) and 1-2 (285.50 mg, 0.990 mmol, 1.2 equiv) in DCE (4 mL) was added ST AB (524.76 mg, 2.475 mmol, 3 equiv) and HOAc (49.56 mg, 0,825 mmol, 1 equiv) at room temperature. The resulting mixture was stirred for overnight at room temperature. The reaction was quenched with water (10 mL) at room temperature. The aqueous layer was extracted with CH2Cl2 (2×10 mL). The resulting mixture was concentrated under reduced pressure. The residue was purified by Prep-TLC (CH2Cl2/MeOH=15:1) to afford 244c (180 mg, 39.41%) as a yellow solid.

### 4. Synthesis of 244

To a stirred solution of 244c (180 mg, 0.350 mmol, 1 equiv) and Pyridine (166.01 mg, 2.100 mmol, 6 equiv) in DCM (5 mL) was added Triphosgene (85.11 mg, 0,287 mmol, 0.82 equiv) at 0° C. The resulting mixture was stirred for 30 min at room temperature. The reaction was quenched by the addition of NaHCO₃ (aq.) (30 mL) at room temperature. The aqueous layer was extracted with CH2Cl2 (2×10 mL). The resulting mixture was concentrated under reduced pressure. The residue was purified by silica gel column chromatography, eluted with CH2Cl2/MeOH=15:1 to afford the crude product (110 mg) as a yellow solid. The crude product (110 mg) was purified by Prep-HPLC with the following conditions (Column: XBridge Prep OBD C18 Column, 30*150 mm, 5 μm; Mobile Phase A: water (10 mmol/L NH4HCO3), Mobile Phase B: ACN; Flow rate: 60 mL/min; Gradient: 28% B to 58% B in 8 min, 58% B; Wave Length: 220 nm; RT1 (min): 7.53) to afford 244 (43.3 mg, 22.07%) as a yellow solid.

LCMS-244 (ES, m/z): [M+H]⁺ 541. H-NMR-244 (400 MHz, DMSO-d6, ppm, δ): 1.05 (s, 3H), 1.77-1.81 (m, 6H), 2.02-2.08 (m, 2H), 2.65-2.68 (m, 1H), 2.72-2.75 (m, 1H), 3.23-3.27 (m, 3H), 3.46 (s, 3H), 3.46-3.52 (m, 2H), 3.73-3.76 (d, 1H), 4.25-4.28 (d, 1H), 7.02 (s, 1H), 7.19-7.21 (d, 1H), 7.31 (s, 1H), 7.42-7.46 (t, 2H), 7.68-7.70 (m, 2H), 7.74 (s, 1H), 8.33 (s, 1H).

### Example 245. Synthesis of Compound 245

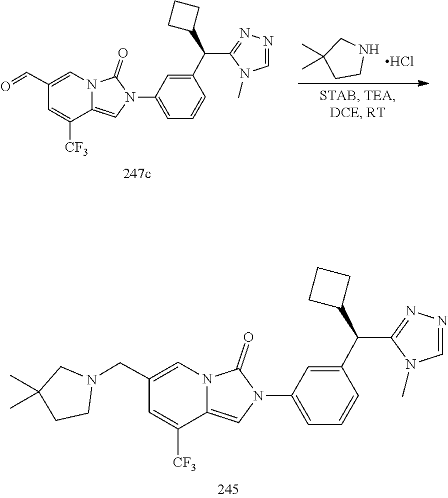

### Synthesis of 245

To a stirred solution of 247c (250 mg, 0.549 mmol, 1 equiv) and 3,3-dimethylpyrrolidine hydrochloride (148.91 mg, 1.098 mmol, 2 equiv) in DCE (5 mL) was added TEA (111.09 mg, 1.098 mmol, 2 equiv) at room temperature under nitrogen atmosphere. The resulting mixture was stirred for 1h at room temperature. To the above mixture was added STAB (232.68 mg, 1.098 mmol, 2 equiv) at room temperature. The resulting mixture was stirred overnight at room temperature. The reaction was quenched with saturated NH4Cl (aq.) (100 mL) at room temperature. The aqueous layer was extracted with DCM (2×20 ml). The organic layers were concentrated under reduced pressure. The residue was purified by Prep-TLC (DCM/MeOH 20:1) to afford the crude product. The crude product (250 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 44% B to 74% B in 8 mii, Wave Length: 220 nm; RT1 (min): 7.32) to afford 245 (159.5 mg, 53.41%) as a yellow solid.

LC-MS: (ES, m/z): [M+H]⁺ 539. H-NMR: (400 MHz, DMSO-d6, δ ppm): 1.05 (s, 6H), 1.52-1.56 (m, 2H), 1.68-1.88 (m, 5H), 2.03-2.13 (m, 1H), 2.28-2.33 (m, 2H), 2.51-2.59 (m, 2H), 3.19-3.25 (m, 1H), 3.38 (s, 2H), 3.43 (s, 3H), 4.25-4.27 (d, 1H), 7.03 (s, 1H), 7.18-7.20 (d, 1H), 7.42 (s, 1H), 7.44-7.46 (m, 1H), 7.66-7.70 (m, 2H), 7.74 (s, 1H), 8.32 (s, 1H).

### Example 246. Synthesis of Compound 246

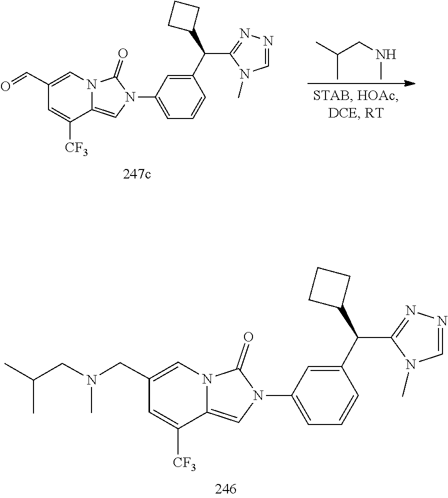

### Synthesis of 246

To a stirred solution of 247c (250 mg, 0,549 mmol, 1 equiv) and methyl(2-methylpropyl)amine (95.69 mg, 1.098 mmol, 2 equiv) in DCE (5 m L) was added HOAc (3.30 mg, 0.055 mmol, 0.1 equiv) and STAB (232.68 mg, 1.098 mmol, 2 equiv) at room temperature under nitrogen atmosphere. The resulting mixture was stirred for overnight at room temperature under. The reaction was quenched with saturated NH4Cl (aq.) (100 mL) at room temperature. The aqueous layer was extracted with DCM (2×20 mL). The organic layers were concentrated under reduced pressure. The residue was purified by Prep-TLC DCM/MeOH 20:1) to afford the crude product. The crude product (250 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 49% B to 79% B in 8 min, Wave Length: 220 nm; RT1 (min): 7.70) to afford 246 (62.3 mg, 21.42%) as a yellow solid.

LC-MS-246: (ES, m/z): [M+H]⁺ 527. H-NMR-246 (400 MHz, DMSO-d6, δ ppm): 0.79-0.93 (d, 6H), 1.68-1.93 (m, 6H), 2.01-2.09 (m, 3H), 3.27-3.33 (m, 3H), 3.43 (s, 3H), 4.25-4.27 (d, 1H), 7.00 (s, 1H), 7.18-7.20 (d, 1H), 7.42 (s, 1H), 7.44-7.46 (m, 1H), 7.67-7.70 (m, 2H), 7.74 (s, 1H), 8.32 (s, 1H).

### Example 247. Synthesis of Compound 247

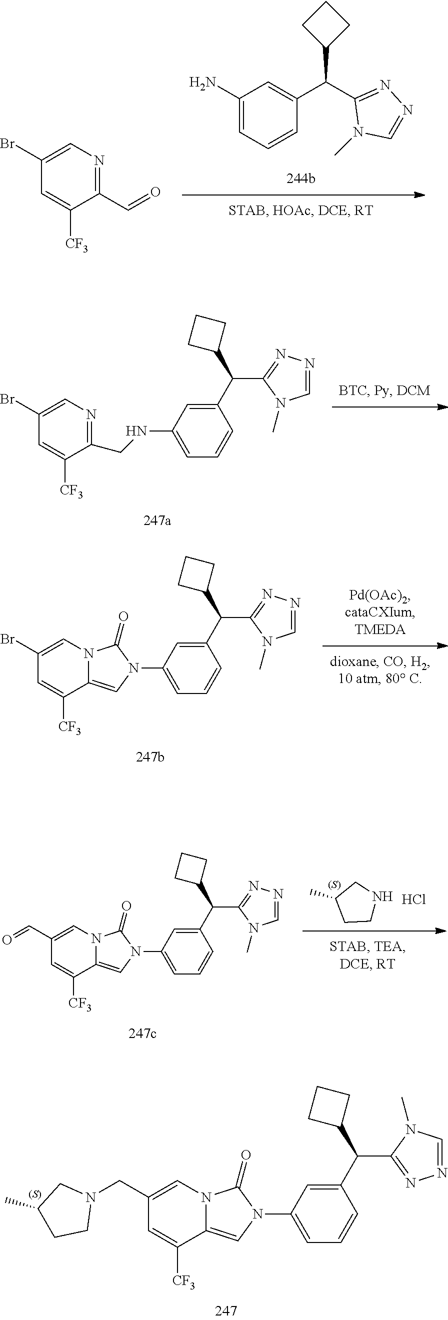

### 1. Synthesis of 247a

To a stirred solution of 21 5a (10.38 g, 40.854 mmol, 1.1 equiv) and 244b (9 g, 37.140 mmol, LOO equiv) in DCE (120 mL) were added STAB (15.74 g, 74.280 mmol, 2 equiv) and HOAc (2.23 g, 37.140 mmol, 1 equiv) at room temperature. The resulting mixture was stirred for overnight at room temperature. The reaction was quenched by the addition of Sat. NH4Cl(aq.) (200 mL) at room temperature. The resulting mixture was extracted with CH2Cl2 (3×350 mL). The resulting mixture was concentrated under reduced pressure. The residue was purified by silica gel column chromatography, eluted with CH2Cl2/MeOH (50:1) to afford 247a (14 g, 74.55%) as a light yellow oil.

### 2. Synthesis of 247b

To a stirred solution of 247a (14 g, 29.146 mmol, 1 equiv) and Pyridine (13.83 g, 174.876 mmol, 6 equiv) in DCM (150 mL) were added Triphosgene (3.03 g, 10.201 mmol, 0.35 equiv) at 0° C. The resulting mixture was stirred for 20 min at room temperature. The reaction was quenched by the addition of Sat. NaHCO₃ (aq.) (200 mL) at room temperature. The resulting mixture was extracted with CH2Cl2 (3×300 mL). The resulting mixture was concentrated under vacuum. The residue was purified by trituration with MTBE (20 mL) to afford 247b (12 g, 77.25%) as a yellow solid.

### 3. Synthesis of 247c

To a solution of 247b (14 g, 27.650 mmol, 1 equiv), TMEDA (6.43 g, 55.300 mmol, 2 equiv) in dioxane (400.00 mL) was added bis(adamantan-1yl)(butyl)phosphane (1.98 g, 5.530 mmol, 0.2 equiv) and Pd(OAc)2 (0.62 g, 2.765 mmol, 0.1 equiv) in an autoclave. After flushing the autoclave three times with CO/H2 (1:1), the mixture was pressurized to 10 atm with CO/H2 (1:1) at 80° C. for overnight. The resulting mixture was concentrated under vacuum. The residue was purified by silica gel column chromatography eluted with CH2Cl2/MeOH (20:1) to afford 247c (12 g, 90.53%) as a yellow solid.

### 4. Synthesis of 247

To a stirred solution of 247c (250 mg, 0.549 mmol, 1.00 equiv) and (3S)-3-methylpyrrolidine hydrochloride (133.51 mg, 1.098 mmol, 2 equiv) in DCE (3 mL) were added TEA (111.09 mg, 1,098 mmol, 2 equiv) at room temperature. The mixture was stirred for 30 min at room temperature. To the above mixture was added STAB (232.68 mg, 1.098 mmol, 2 equiv) at room temperature. The resulting mixture was stirred for overnight at room temperature. The reaction was quenched by the addition of sat. NH4Cl (aq.) (7m-L) at room temperature. The resulting mixture was extracted with CH2Cl2 (3×10 mL). The resulting mixture was concentrated under reduced pressure. The crude product (143 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 34% B to 64% B in 8 min, 64% B; Wave Length: 220 nm; RT1 (min): 7.78) to afford 247 (101.3 mg, 34.97%) as a yellow solid.

LC-MS: (ES, m/z): [M+H]+: 525. H-NMR: (400 MHz, DMSO-d6, ppm): δ 0.97-1.03 (m, 3H), δ 1.22-1.35 (m, 1H), δ 1.61-1.85 (m, 5H), δ 1.86-2.13 (m, 3H), δ 2.15-2.22 (m, 1H), δ 2.52 (s, 1H), δ 2.53-2.56 (d, 1H), δ 2.72-2.76 (m, 1H), δ 3.15-7.23 (m, 1H), δ 3.35 (s, 2H), δ 3.45 (s, 3H), δ 4.25-4.27 (d, 1H), δ 7.00 (s, 1H), δ 7.21-7.23 (d, 1H), δ 7.30 (s, 1H), δ 7.41-7.45 (m, 1H), δ 7.64-7.68 (m, 1H), δ 7.73 (s, 1H), δ 8.32 (s, 1H).

### Example 248. Synthesis of Compound 248

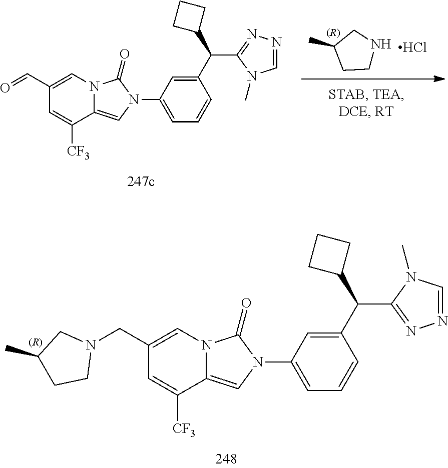

### Synthesis of 248

To a stirred solution of 247c (250 mg 0.549 mmol, 1.00 equiv) and (3R)-3-methylpyrrolidine hydrochloride (133.51 mg, 1.098 mmol, 2 equiv) in DCE (3 mL) were added TEA (111.09 mg, 1.098 mmol, 2 equiv) at room temperature. The mixture was stirred for 30 min at room temperature. To the above mixture was added STAB (232.68 mg, 1.098 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×15 mL). The resulting mixture was concentrated under reduced pressure. The crude product (120 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 34% B to 62% B in 8 min, 62% B; Wave Length: 220 nm; RT1 (min): 7.83) to afford 248 (81.7 mg, 28.09%) as a yellow solid.

LCMS: (ES, m/z): [M+H]+: 525. H-NMR: (400 MHz, DMSO-d6, ppm): δ 0.97-1.03 (m, 3H), δ 1.22-1.35 (m, 1H), δ 1.61-1.85 (m, 5H), δ 1.86-2.13 (m, 3H), δ 2.15-2.22 (m, 1H), δ 2.52 (s, 1H), δ 2.53-2.56 (d, 1H), δ 2.72-2.76 (m, 1H), δ 3.15-7.23 (m, 1H), δ 3.35 (s, 2H), δ 3.45 (s, 3H), δ 4.25-4.27 (d, 1H), δ 7.00 (s, 1H), δ 7.21-7.23 (d, 1H), δ 7.30 (s, 1H), δ 7.41-7.45 (m, 1H), δ 7.64-7.68 (m, 2H), δ 7.73 (s, 1H), δ 8.32 (s, 1H)

### Example 249. Synthesis of Compound 249

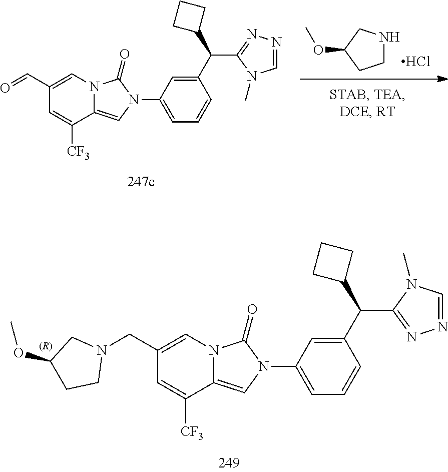

### Synthesis of 249

To a stirred solution of 247c (250 mg, 0,549 mmol, 1.00 equiv) and (3R)-3-methoxypyrrolidine hydrochloride (151.07 mg, 1,098 mmol, 2 equiv) in DCE (3 mL) were added TEA (111.09 mg 1.098 mmol, 2 equiv) at room temperature. The mixture was stirred for 30 min at room temperature. To the above mixture was added STAB (232.68 mg, 1,098 mmol, 2 equiv) at room temperature. The resulting mixture was stirred for overnight at room temperature. The reaction was quenched by the addition of sat. NH4Cl (aq.) (5 mL) at room temperature. The resulting mixture was extracted with CH2Cl2 (3×10 ml). The resulting mixture was concentrated under reduced pressure. The crude product (100 mg) was purified by Prep-HPLC with the following conditions (Column: YMC-Actus Triart C18 ExRS, 30*150 mm, 5 μm; Mobile Phase A: Water (10 mmol/L NH-4HCO3), Mobile Phase B: ACN; Flow rate: 60 mL/min; Gradient: 28% B to 51% B in 11 min, 51% B; Wave Length: 220 nm; RT1 (min): 10.68) to afford 249 (69.5 mg, 23.19%) as a yellow solid.

LC-MS: (ES, m/z): [M+H]+: 541. H-NMR: (400 MHz, DMSO-d6, ppm): δ 1.60-1.72 (m, 2H), δ 1.73-1.85 (m, 4H), δ 1.93-2.15 (m, 1H), δ 2.51-2.53 (d, 1H), δ 2.52-2.54 (m, 2H), δ 2.54-2.57 (m, 1H), δ 2.71-2.73 (d, 1H), δ 3.16 (s, 3H), δ 3.21-3.23 (d, 1H), δ 3.35-3.36 (m, 2H), δ 3.58 (s, 3H), δ 3.85-3.87 (d, 1H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 2H), δ 7.72 (s, 1H), δ 8.33 (s, 1H).

### Example 250. Synthesis of Compound 250

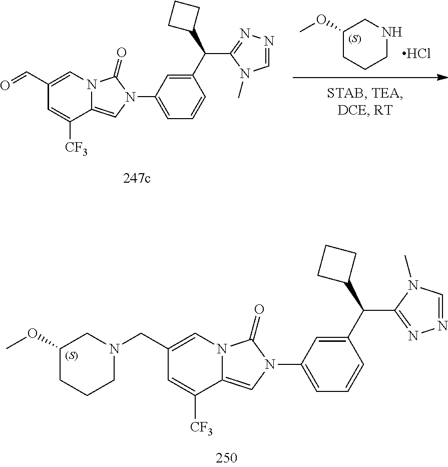

### Synthesis of 250

To a stirred solution of 247c (250 mg, 0.549 mmol, 1 equiv) and (3S)-3-methoxypiperidine hydrochloride (126.44 mg, 1.098 mmol, 2 equiv) in DCE (3 mL) was added TEA (111.09 mg, 1.098 mmol, 2 equiv) at room temperature. The mixture was stirred for 30 min at room temperature. To the above mixture was added STAB (232.68 mg, 1.098 mmol, 2 equiv) at room temperature. The resulting mixture was stirred for overnight at room temperature. The reaction was quenched by the addition of sat. NH4Cl (aq.) (5 mL) at room temperature. The resulting mixture was extracted with EtOAc (3×10 mL). The resulting mixture was concentrated under reduced pressure. The crude product (150 mg) was purified by Prep-HPLC with the following conditions (Column: YMC-Actus Triart C18 ExRS, 30*150 mm, Sim; Mobile Phase A: Water (10 mmol/L NH4H CO3), Mobile Phase B: ACN; Flow rate: 60 mL/min; Gradient: 38% B to 63% B in 8 min, 63% B; Wave Length: 220 nm; RT1 (min): 7.12) to afford 250 (111.7 mg, 36.36%) as a yellow solid.

LC-MS: (ES, m/z): [M+H]+: 555. H-NMR: (400 MHz, DMSO-d6, ppm): δ 1.02-1.13 (m, 1H) S 1.28-1.32 (m, 1H), δ 1.61-1.75 (m, 2H), δ 1.76-1.89 (m, 4H), δ 1.90-1.98 (m, 2H), δ 2.01 (s, 1H), δ 2.14-2.46 (d, 1H), δ 2.62 (s, 1H), δ 2.90 (s, 1H), δ 3.56 (s, 5H), δ 3.32-3.36 (m, 2H), δ 3.45 (s, 3H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 2H), δ 7.75 (s, 1H), δ 8.35 (s, 1H).

### Example 251. Synthesis of Compound 251

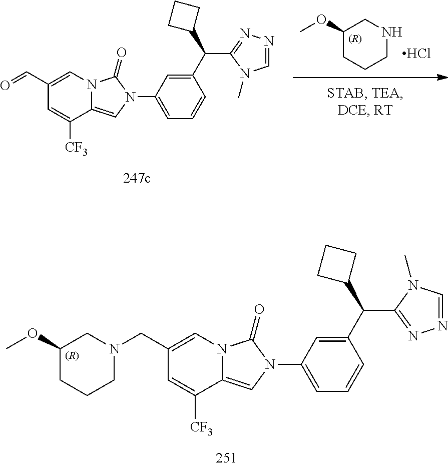

### Synthesis of 251

To a stirred solution of 247c (250 mg 0.549 mmol, 1 equiv) and (3R)-3-methoxypiperidine hydrochloride (126.44 mg, 1.098 mmol, 2 equiv) in DCE (3 mL) was added TEA (111.09 mg, 1.098 mmol, 2 equiv) at room temperature. The mixture was stirred for 30 min at room temperature. To the above mixture was added STAB (232.68 mg, 1.098 mmol, 2 equiv) at room temperature. The resulting mixture was stirred for overnight at room temperature. The reaction was quenched by the addition of sat. N1-4Cl (aq.) (5 mL) at room temperature. The resulting mixture was extracted with EtOAc (3×10 ML). The resulting mixture was concentrated under reduced pressure. The crude product (89 mg) was purified by Prep-HPLC with the following conditions (Column: YMC-Actus Triart C18 ExRS, 30*150 mm, 5 μm; Mobile Phase A: Water (10 mmol/L NH4HCO3), Mobile Phase B: ACN; Flow rate: 60 mL/min; Gradient: 38% B to 63% B in 8 min, 63% B; Wave Length: 220 nm; RT1 (min): 7.12) to afford 251 (71.0 mg, 23.20%) as a yellow solid.

LC-MS: (ES, m/z): [M+H]+: 555. H-NMR: (400 MHz, DMSO-d6, ppm): δ 1.02-1.13 (m, 1H) δ 1.28-1.32 (m, 1H), δ 1.61-1.75 (m, 2H), δ 1.76-1.89 (m, 4H), δ 1.90-1.98 (m, 3H), δ 2.01 (s, 1H), δ 2.14-2.46 (d, 1H), δ 2.62 (s, 1H), δ 3.56 (s, 5H), δ 3.32-3.36 (m, 2H), δ 3.45 (s, 3H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 2H), δ 7.75 (s, 1H), δ 8.35 (s, 1H).

### Example 252. Synthesis of Compound 252

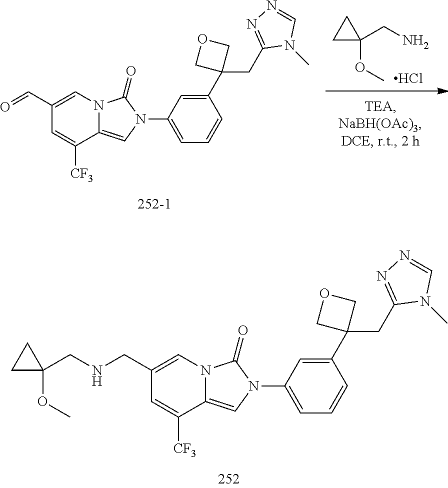

### Synthesis of 252

To a solution of 252-1 (0.1 g, 218 umol, 1 equiv) and (1-methoxycyclopropyl)methanamine (90.2 mg, 655 umol, HCl salt, 3 equiv) in DCE (1.5 mL) was added TEA (121 uL, 874 umol, 4 equiv). The mixture was stirred at 20° C. for 1 hr. Then it was added NaBH(OAc)3 (231.68 mg, 1.09 mmol, 5 equiv). The mixture was stirred at 20° C. for 1 hr. The reaction was quenched by the addition of water (10 mL) and extracted with CH2Cl2 (2×30 ml). The combined organic layers were dried with anhydrous sodium sulfate, filtered and the filtrate was concentrated under reduced pressure. The residue was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 10% B to 50% B in 8 min; Wave Length: 220 nm; RT (min): 8.0) to afford 252 (17 mg, 14% yield) as a yellow solid.

LCMS: (ES, m/z): [M+H]⁺ 543.3. H-NMR: (400 MHz, DMSO-d6) δ 8.18 (d, J=12 Hz, 2H), 7.75 (d, J=6.8 Hz, 1H), 7.70 (s, 1H), 7.41-7.37 (m, 2H), 7.29 (s, 1H), 7.15 (s, 1H), 6.88 (d, J=7.6 Hz, 1H), 4.96-4.89 (m, 4H), 3.59 (s, 2H), 3.53 (s, 2H), 3.18 (s, 3H), 2.96 (s, 3 H), 2.68 (s, 2H), 0.62-0.68 (m, 2H), 0.47 (d, J=2.0 Hz, 2H)

### Example 253. Synthesis of Compound 253

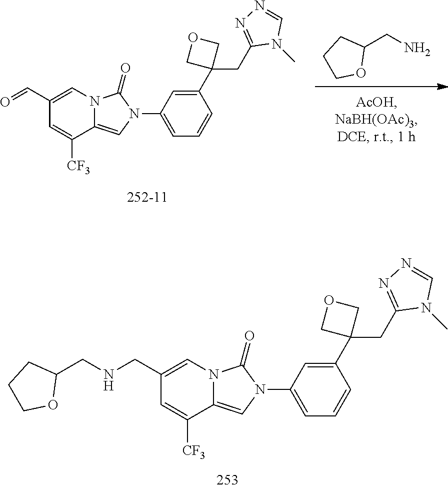

### Synthesis of 253

To a solution of 252-11 (100 mg, 218 umol, 1 equiv) and tetrahydrofuran-2-ylmethanamine (135 uL, 1.31 mmol, 6 equiv) in DCE (0.5 mL) was added AcOH (13.1 mg, 218 umol, 1 equiv). The mixture was stirred at 20° C. for 0.5 hr. Then it was added NaBH(OAc)3 (92.6 mg, 437 umol, 2 equiv). The mixture was stirred at 20° C. for 0.5 h. The reaction was quenched by the addition of water (10 mL) and extracted with CH2Cl2 (2×30 ml). The combined organic layers were dried with anhydrous sodium sulfate, filtered and the filtrate was concentrated under reduced pressure. The residue was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 20% B to 60% B in 8 min; Wave Length: 220 nm; RT (min): 9.0) to afford 253 (15 mg, 13% yield) as a yellow solid.

LCMS: (ES, m/z): [M+H]⁺ 543.3.

H-NMR: (400 MHz, DMSO-d6) δ 8.18 (d, J=10.8 Hz, 2H), 7.73-7.78 (m, 1H), 7.69 (s, 1 H), 7.40 (d, J=3.6 Hz, 2H), 7.29 (s, 1H), 7.13 (s, 1H), 6.88 (d, J=8.0 Hz, 1H), 4.96-4.89 (m, 4H), 3.92-3.84 (m, 2H), 3.76-3.70 (m, 2H) 3.63-3.58 (m, 3H), 3.55 (s, 2H), 3.53 (s, 2 H), 2.97 (s, 3H), 2.91-2.86 (m, 1H), 1.91-1.77 (m, 4H), 1.54-1.51 (m, 1H).

### Example 254. Synthesis of Compound 254

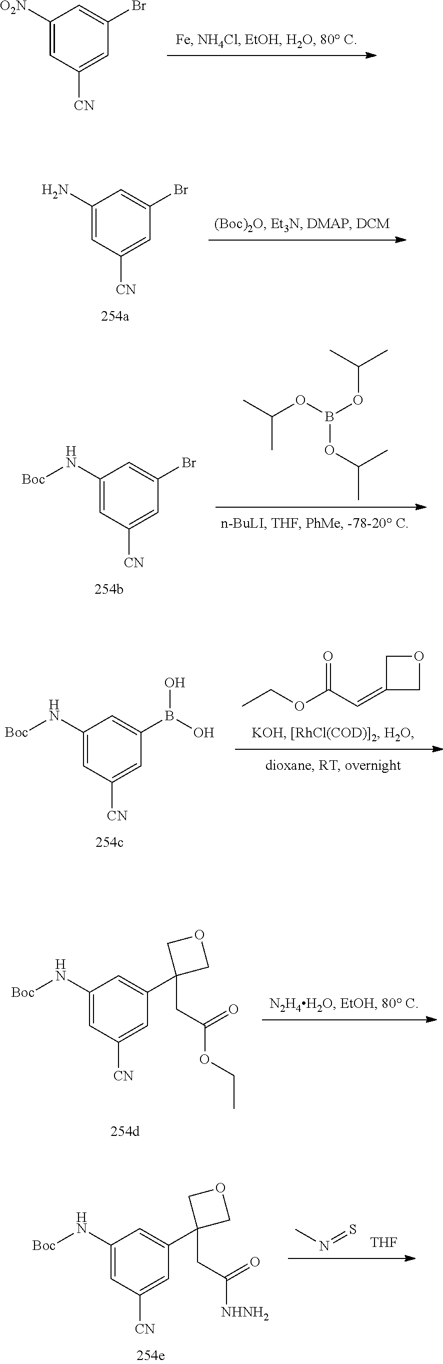

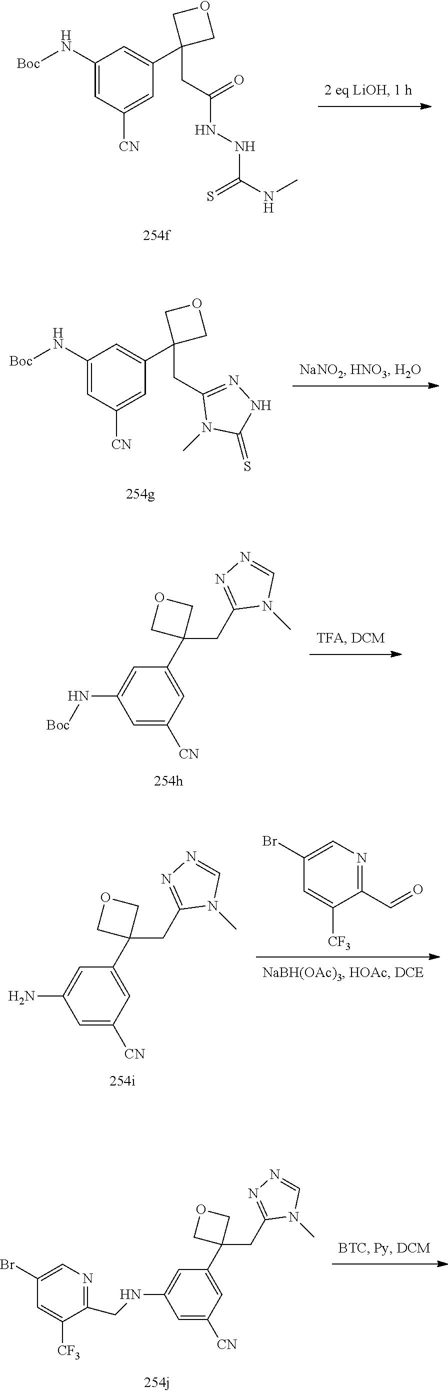 [SMILES: CNC(=S)NNC(=O)CC1(c2cc(C#N)cc(NC(=O)OC(C)(C)C)c2)COC1 ; Cn1c(CC2(c3cc(C#N)cc(NC(=O)OC(C)(C)C)c3)COC2)n[nH]c1=S ; Cn1cnnc1CC1(c2cc(C#N)cc(NC(=O)OC(C)(C)C)c2)COC1 ; Cn1cnnc1CC1(c2cc(N)cc(C#N)c2)COC1 ; O=Cc1ncc(Br)cc1C(F)(F)F ; Cn1cnnc1CC1(c2cc(C#N)cc(NCc3ncc(Br)cc3C(F)(F)F)c2)COC1]

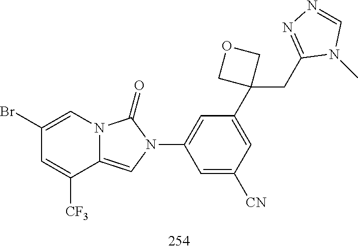 [SMILES: Cn1cnnc1CC1(c2cc(C#N)cc(-n3cc4c(C(F)(F)F)cc(Br)cn4c3=O)c2)COC1]

### 1. Synthesis 254a

Into a 2 L 3-necked round-bottom flask were added 3-bromo-5-nitrobenzonitrile (100 g, 440,496 mmol, 1 equiv), Fe (73.80 g, 1321.488 mmol, 3 equiv), NH4Cl (117.81 g 2202480 mol, 5 equiv), EtOH (800 mL) and H2O (200 mL) at room temperature. The resulting mixture was stirred overnight at 80° C. under nitrogen atmosphere. The resulting mixture was filtered, the filter cake was washed with EtOAc (2×100 mL). The filtrate was concentrated under reduced pressure. The residue was purified by trituration with water (500 m) This resulted in 254a (88 g, 9632%) as a light yellow so d.

### 2. Synthesis of 254b

Into a 2t 3-necked round-bottom flask were added 254a (88 g 446.621 m mol, 1 equiv), (Boc)2O (99.42 g, 455.553 ml, 1.02 equiv), Et3N (90.39 g, 893.242 mmol, 2 equiv), DMAP (5.46 m, 44.662 mmol, 0.1 equiv) and DCM (880 mL) at room temperature. The resulting mixture was stirred overnight at room temperature under nitrogen atmosphere. The reaction was quenched with water (1000 mL) at room temperature. The resulting mixture was, extracted with DCM (3×2010 mL). The organic layers were concentrated under reduced pressure. The residue was purified by silica gel column chromatography, eluted with E/EA (10:1) to afford 254h (21 g, 15.03%) as a light yellow solid

### 3, Synthesis of 254c

To a stirred solution of 254b (15 g, 50.479 mmol, 1 equiv) and triisopropyl borate (408.07 mg, 2.0120 mmol, 1.2 equiv) in THE (30 mL)/Toluene (120 mL) was added n-BuLi (60.58 ml, 151.437 mmol, 3 equiv, 2.5M) at −78° C. under nitrogen atmosphere. The reaction mixture was stirred at −20° C. for 30 mins under nitrogen atmosphere. The reaction was quenched with saturated NH4Cl (aq.) (300 mL). The resulting mixture was concentrated under reduced pressure. The residue was purified by reverse flash chromatography with the following conditions: column, C18 silica gel; mobile phase, MeCN in water, 10% to 40% gradient in 10 min; detector, LV 254 nm. This resulted in 254c (4 g, 28.72%) as a yellow solid.

### 4, Synthesis of 254d

To a stirred solution of KOH (16.79 mL, 25.184 mmol, 12 equiv) in 1120 (15 mL)/dioxane (55 mL) was added [Rh(COD)Cl]2 (0.52 g, 1.049 mmol, 0.05 equiv) at room temperature under nitrogen atmosphere. The resulting mixture was stirred for 1 h at room temperature under nitrogen atmosphere. To the above mixture was added 254c (5.5 g, 20.987 mmol, 1 equiv) and ethyl 2-(oxetan-3-ylidene)acetate (5.97 g, 41.974 mmol, 2 equiv) at room temperature. The resulting mixture was stirred overnight at room temperature under nitrogen atmosphere. The mixture was neutralized to pH 7 with saturated NH4Cl (aq.) (300 mL). The resulting mixture was extracted with EtOAc (3×200 mL). The combined organic layers were washed with water (2×50 mL), dried over anhydrous Na2SO4. After filtration, the filtrate was concentrated under reduced pressure. The residue was purified by silica gel column chromatography, eluted with PE/EA (12:1) to afford 254d (2.4 g, 30.14%) as a light yellow solid.

### 5. Synthesis of 254e

Into a 100 mL 3-necked round-bottom flask were added 254d (2.4 g, 6.659 mmol, 1 equiv) and EtOH (24 mL) and hydrazine hydrate (98%)(173.62 mg, 3.475 mmol, 25 equiv) at room temperature. The resulting mixture was stirred overnight at 80° C. under nitrogen atmosphere. The reaction was quenched by the addition of water (100 mL) at room temperature. The resulting mixture was extracted with DCM (3×150 mL). The combined organic layers were dried over anhydrous Na2SO4. After filtration, the filtrate was concentrated under reduced pressure. The residue was purified by silica gel column chromatography, eluted with DCM/MeOH (20:1) to afford 254e (1.4 g, 57.66%) as a pink solid.

### 6. Synthesis of 254f

To a stirred solution of 254e (1.35 g, 3,897 mmol, 1 equiv) in tetrahydrofuran (20 mL) was added methyl isothiocyanate (712.34 mg, 9.742 mmol, 2.5 equiv) at room temperature. The resulting mixture was stirred overnight at room temperature. The reaction was quenched with water (50 mL) at room temperature. The aqueous layer was extracted with EtOAc (3×20 mL). The organic layers were concentrated under reduced pressure. The residue was purified by Prep-TLC (DCM/MeOH 20:1) to afford 254f (1.05 g, 61.65%) as a light yellow solid.

### 7. Synthesis of 254g

To a stirred mixture of 254f (1 g, 2.384 mmol, 1 equiv) in 1H2O (5 mL) was added LiOH (228.37 mg, 9,535 mmol, 4.00 equiv) at room temperature. The resulting mixture was stirred for 2h at room temperature. The mixture was acidified to pH 4 with HCl(aq.). The aqueous layer was extracted with DCM/MeOH (10:1) (3×20 mL). The resulting mixture was concentrated under reduced pressure. The residue was purified by Prep-TLC (DCM/MeOH 12:1) to afford 254g (540 mg, 54.17%) as a white solid.

### 8. Synthesis of 254h

To a stirred mixture of 254g (520 mg, 1.295 mmol, 1 equiv) and NaNO2 (893.61 mg, 12.950 mmol, 10 equiv) in H2O (8 mL) were added HNO3 (13 mL, 12.950 mmol, 10 equiv, 1M) dropwise at 0° C. The resulting mixture was stirred for 1h at 0° C. The mixture was acidified to pH 7 with saturated NaHCO₃ (aq.). The aqueous layer was extracted with DCM/MeOH (10:1) (3×20 mL). The resulting mixture was concentrated under reduced pressure to afford 254h (400 mg, 75.24%) as a yellow solid.

### 9. Synthesis of 254i

To a stirred mixture of 254h (410 mg, 1.110 mmol, 1 equiv) in DCM (10 mL) was added TFA (1 mL) at room temperature. The resulting mixture was stirred for 2h at room temperature. The reaction was quenched by the addition of NaHCO₃ (aq.) (20 mL) at room temperature. The aqueous layer was extracted with DCM/MeOH (10:1) (4×20 mL). The resulting mixture was concentrated under reduced pressure to afford 254i (300 mg, 89.33%) as a yellow oil,

### 10. Synthesis of 254j

To a stirred solution of 254i (280 mg, 1.040 mmol, 1 equiv) and 5-bromo-3-(trifluoromethyl)picolinaldehyde (396.14 mg, 1.560 mmol, 1.5 equiv) in DCE (5 mL) was added NaBH(OAc)3 (440.71 mg, 2.080 mmol, 2 equiv) and HOAc (6.24 mg, 0.104 mmol, 0.1 equiv) at room temperature under nitrogen atmosphere. The resulting mixture was stirred overnight at room temperature. The reaction was quenched with NaHCO3 (aq.) (30 mL) at room temperature. The aqueous layer was extracted with DCM (2×20 mL). The organic layers were concentrated under reduced pressure. The residue was purified by Prep-TLC (DCM/MeOH 20:1) to afford 254j (120 mg, 20.70%) as a light yellow solid.

### 11. Synthesis of 254

To a stirred solution of 254j (110 mg 0,217 mmol, 1 equiv) and Pyridine (171.51 mg, 2.170 mmol, 10 equiv) in DCM (5 mL) was added Triphosgene (32.17 mg, 0.108 mmol, 0.5 equiv) at room temperature under nitrogen atmosphere. The reaction was quenched with saturated NaHCO3 (aq.) (30 mL) at room temperature. The aqueous layer was extracted with DCM (2×20 mal). The resulting mixture was concentrated under vacuum. The product was precipitated by the addition of MeOH. This resulted in 254 (64.0 mg, 54.96%) as a yellow solid.

LC-MS: (ES, m/z): [M+H]⁺ 533, H-NMR: (400 MHz, DMSO-d6, δ ppm): 3.23 (s, 3H), 3.41 (s, 2H), 4.90-4.96 (m, 4H), 7.21 (s, 1H), 7.60-7.63 (m, 2H), 7.99 (s, 1H), 8.06 (s, 1H), 8.24-8.26 (m, 2H).

### Example 255. Synthesis of Compound 255

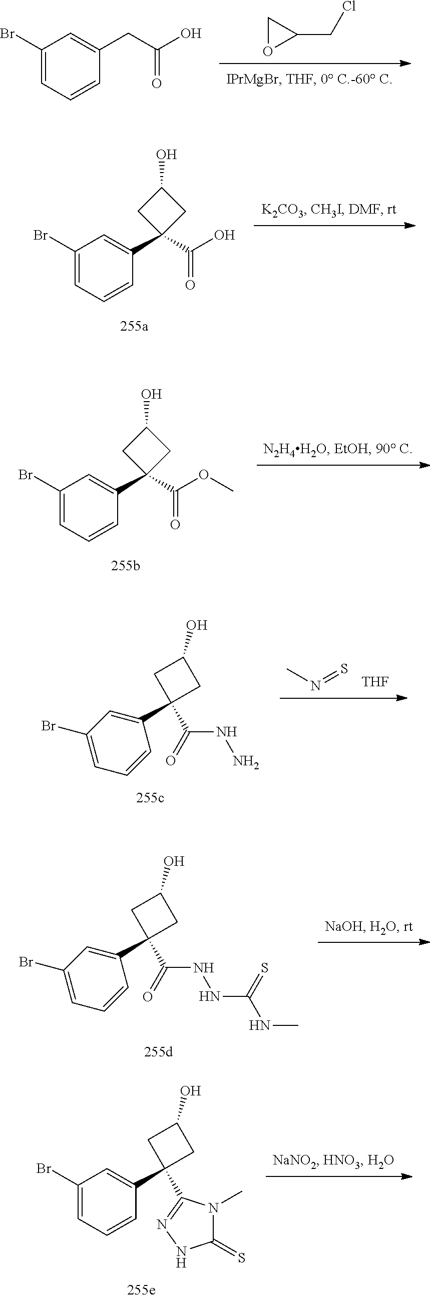

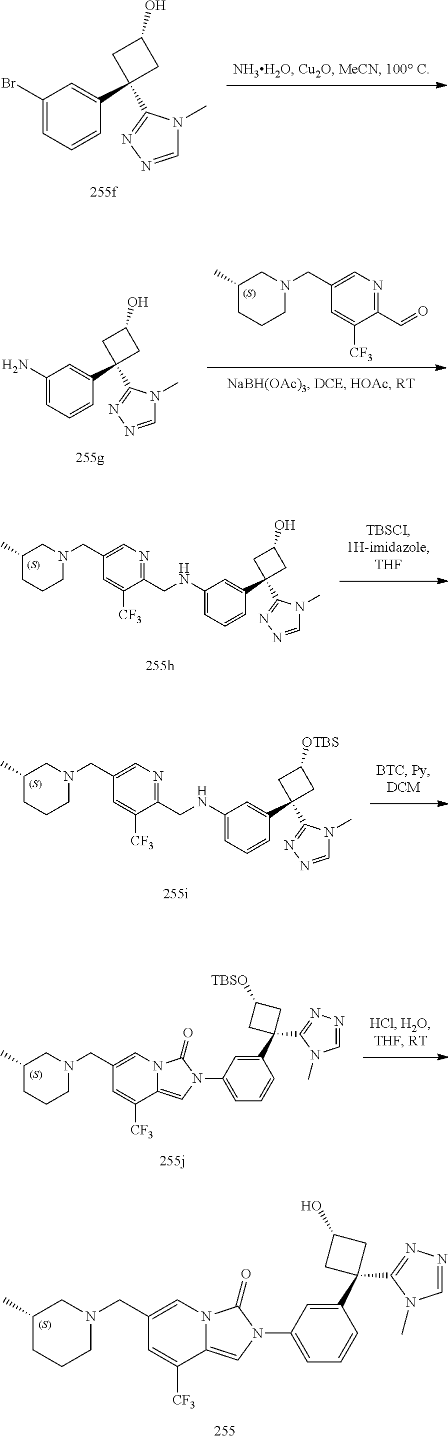

### 1. Synthesis of 255a

To a stirred solution of M-bromophenylacetic acid (100 g, 465.017 mmol, 1 equiv) in THF (700 mL) was added i-PrMgBr (511.52 mL, 511.52 mmol, 1.1 equiv) dropwise at 0° C. under nitrogen atmosphere. To the above mixture was added epichlorohydrin (64.54 g, 697.525 mmol, 1.5 equiv) dropwise at 0° C. The resulting mixture was stirred for additional 45 min at 0° C. To the above mixture was added i-PrMgBr (511.52 mL, 511.52 mmol, 1.1 equiv, 1M) dropwise at 0° C. The resulting mixture was slowly heated to at 60° C. and stirred for 16h. The reaction was quenched with saturated NH4Cl (aq.) (3.5 L) at room temperature. The aqueous layer was extracted with EtOAc (3×1 L). The resulting mixture was concentrated under vacuum. The residue was purified by reverse flash chromatography with the following conditions: column, C18 silica gel; mobile phase, MeCN in water, 10% to 50% gradient in 10 min; detector, UV 220 nm. This resulted in 255a (34 g, 21.58%) as a colorless oil.

### 2. Synthesis of 255b

To a stirred solution of 255a (16 g, 59.017 mmol, 1 equiv) and C131 (25.13 g, 177.051 mmol, 3 equiv) in DMF (200 mL) were added K2CO3 (24.47 g, 177,051 mmol, 3 equiv) at room temperature under nitrogen atmosphere. The resulting mixture was stirred overnight at room temperature. The resulting mixture was diluted with water (500 mL). The aqueous layer was extracted with EtOAc (2×500 mL) The resulting mixture was concentrated under vacuum. The residue was purified by reverse flash chromatography with the following conditions: column, C18 silica gel; mobile phase, MeCN in water, 20% to 70% gradient in 20 min; detector, UV 220 nm. This resulted in 255b (1.4 g, 7.90%) as an off-white solid.

### 3, Synthesis of 255c

To a stirred solution of 255b (1.4 g, 4.910 mmol, 1 equiv) in EtOH (20 mL) was added hydrazine hydrate (98%)(2.46 g, 49.100 mmol, 10 equiv) at room temperature. The resulting mixture was stirred for overnight at 90° C. The resulting mixture was diluted with water (100 mL). The aqueous layer was extracted with DCM/MeOH=10:I (2×20 mL). The resulting mixture was concentrated under vacuum. This resulted in 255c (1.2 g, 78.86%) as a light yellow solid.

### 4. Synthesis of 255d

To a stirred solution of 255c (1.2 g, 4.208 mmol, 1 equiv) in tetrahydrofuran (15 mL) was added methyl isothiocyanate (0.77 g, 10,520 mmol, 2.5 equiv) at room temperature. The resulting mixture was stirred overnight at room temperature. The resulting mixture was diluted with water (100 mL). The aqueous layer was extracted with DCM/MeOH=10:1 (3×20 mL). The resulting mixture was concentrated under vacuum. This resulted in 255d (1.5 g, 99.49%) as an off-white solid,

### 5, Synthesis of 255e

To a stirred solution of NaOH (1.34 g, 33.496 mmol, 8 equiv) in 1120 (35 mL) was added 255d (1.5 g, 4.187 mmol, 1 equiv) at room temperature. The resulting mixture was stirred overnight at room temperature. The mixture was acidified to pH 6 with HCl (aq.). The aqueous layer was extracted with DCM/MeOH=0:1 (2×20 mL). The resulting mixture was concentrated under vacuum. This resulted in 255e (1.5 g, 94.76%) as an off-white solid.

### 6. Synthesis of 255f

To a stirred solution of 255e (1.5 g 4.409 mmol, 1 equiv) and NaNO2 (3.04 g, 44.090 mmol, 10 equiv) in H2O (20 mL) was added 11N03 (44.09 mL, 44.090 mmol, 10 equiv, 1M) dropwise at 0° C. The resulting mixture was stirred for overnight at room temperature. The mixture was basified to pH 7 with NaHCO₃ (aq.). The aqueous layer was extracted with DCM (3×20 mL). The resulting mixture was concentrated under vacuum. This resulted in 255f (13 g, 86.11%) as a light yellow solid.

### 7. Synthesis of 255g

Into a 20 mL pressure tank reactor was added 255f (500 mg, 1.622 mmol, 1 equiv), Cu2O (46.43 mg, 0,324 mmol, 0.2 equiv), NH3·H2O (5 mL) and MeCN (5 mL) at room temperature. The resulting mixture was stirred overnight at 100° C. The mixture was allowed to cool down to room temperature. The resulting mixture was concentrated under vacuum. The residue was purified by Prep-TLC (DCM/MeOH 20:1) to afford 255g (350 mg, 79.47%) as a brown light solid.

### 8. Synthesis of 255h

To a stirred solution of 255g (350 mg, 1.433 mmol, 1 equiv) and I-2 (533.22 mg, 1.863 mmol, 1.3 equiv) in DCE (8 mL) was added NaBH(OAc)3 (607.28 mg, 2,866 mmol, 2 equiv) and HOAc (8.60 mg, 0,143 mmol, 0.1 equiv) at room temperature. The resulting mixture was stirred overnight at room temperature. The reaction was quenched with water (50 mL) at room temperature. The aqueous layer was extracted with DCM (3×30 mL). The organic layers were concentrated under reduced pressure. The residue was purified by Prep-TLC (DCM/MeOH 12:1) to afford 255h (550 mg, 70.87%) as a yellow solid.

### 9. Synthesis of 255i

To a stirred solution of 255h (550 mg, 1.069 mmol, 1 equiv) and 1H-imidazole (363.80 mg, 5.345 mmol, 5 equiv) in THE (20 mL) was added TBSCl (483.27 mg, 3,207 mmol, 3 equiv) at room temperature under nitrogen atmosphere. The resulting mixture was stirred overnight at 70° C. The reaction was quenched with water (80 mL) at room temperature. The aqueous layer was extracted with EtOAc (3×20 mL). The organic layers were concentrated under reduced pressure. The residue was purified by Prep-TLC (DCM/MeOH 15:1) to afford 255i (500 mg, 70.67%) as an off-white solid.

### 10. Synthesis of 255j

To a stirred solution of 255i (500 mg, 0.795 mmol, 1 equiv) and Pyridine (628.92 mg, 7.950 mmol, 10 equiv) in DCM (10 mL) was added Triphosgene (117.96 mg, 0.398 mmol, 0.50 equiv) at room temperature under nitrogen atmosphere. The resulting mixture was stirred for 10 min at room temperature. The reaction was quenched with NaHCO₃ (aq.) (50 mL) at room temperature. The aqueous layer was extracted with DCM (3×30 mL). The resulting mixture was concentrated under vacuum. The crude product was used in the next step directly without further purification. This resulted in 255j (400 mg, 69.14%) as a yellow solid.

### 11. Synthesis of 255

To a stirred solution of 255j (380 mg, 0.580 mmol, 1 equiv) and H2 O (6 mL, 2.900 mmol) in THF (2 mL) was added HCl (2 mL, 1M) at room temperature. The resulting mixture was stirred for 2h at room temperature. The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 55% B in 9 min, Wave Length: 220 am; RT1 (min): 8.27) to afford 255 (225.8 mg, 71.26%) as a yellow solid.

LC-MS: (ES, m/z): [M+H]⁺ 541. H-NMR: (400 MHz, DMSO-d6, δ ppm): 0.81-0.93 (m, 4H), 1.39-1.41 (m, 1H), 1.41-1.65 (m, 4H), 1.85-1.91 (m, 1H), 2.73-2.78 (m, 4H), 3.05-3.10 (m, 2H), 3.22 (s, 3H), 3.24 (s, 2H), 4.27-4.32 (m, 1H), 5.30-5.32 (d, 1H), 6.99 (s, 1H), 7.23-7.25 (d, 1H), 7.38 (s, 1H), 7.46-7.50 (m, 1H), 7.64-7.66 (m, 2H), 7.80 (s, 1H), 8.29 (s, 1H).

### Example 256. Synthesis of Compound 256

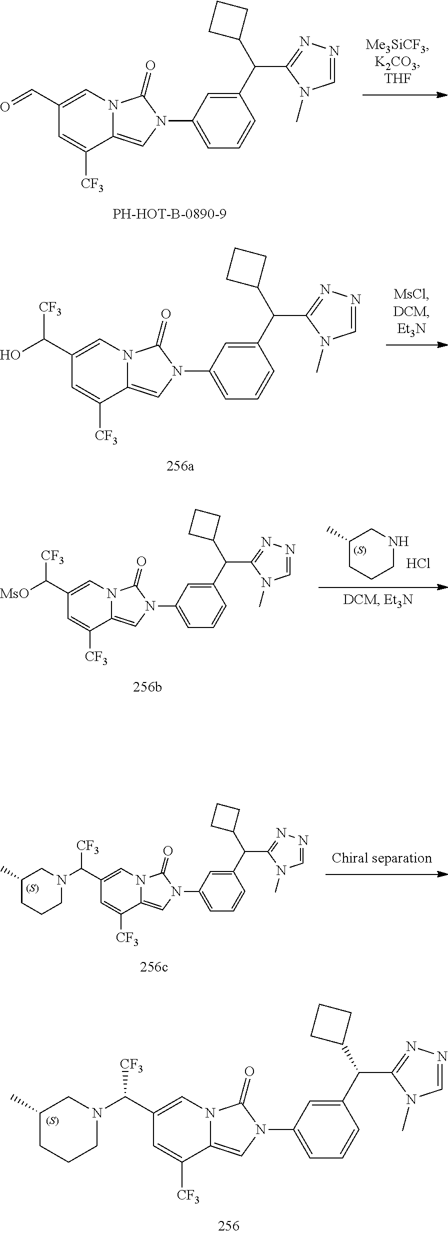

### 1. Synthesis of 256a

To a stirred mixture of 247c (6 g, 13.174 mmol, 1 equiv) and K2CO3 (018 g, 1.317 mmol, 0.1 equiv) in THE (120 mL) was added TMSCF3 (3.75 g, 26,348 mmol, 2 equiv) dropwise at room temperature under nitrogen atmosphere. The resulting mixture was stirred for overnight at room temperature under nitrogen atmosphere. The resulting mixture was diluted with water (300 mL). The resulting mixture was extracted with EtOAc (2×100 mL). The combined organic layers were concentrated under reduced pressure. The residue was purified by reverse flash chromatography with the following conditions: column, C18 silica gel; mobile phase, MeCN in water (10 mmol/L NH4HCO3), 10% to 65% gradient in 30 min; detector, UV 254 nm. This resulted in 256a (570 mg, 8.23%) as a yellow solid,

### 2. Synthesis of 256b

To a stirred solution of 256a (570 mg, 1.085 mmol, 1 equiv) and Et3N (219.54 mg, 2.170 mmol, 2 equiv) in DCM (5 mL) was added MsCl (149.10 mg, 1.302 mmol, 1.2 equiv) dropwise at 0° C. The resulting mixture was stirred for 2 h at 0° C. The resulting mixture was concentrated under reduced pressure. This resulted in 256b (660 mg, crude) as a yellow solid,

### 3. Synthesis of 256c

To a stirred mixture of 256b (660 mg, 1.094 mmol, 1 equiv) and (3S)-3-methylpiperidine hydrochloride (296.66 mg, 2.188 mmol, 2 equiv) in DCM (5 mL) was added Et3N (331.98 mg, 3.282 mmol, 3 equiv) at room temperature. The resulting mixture was stirred for 3 h at room temperature. The resulting mixture was diluted with water (10 mL). The resulting mixture was extracted with CH2Cl2 (3×15 mL). The combined organic layers were concentrated under reduced pressure. The residue was purified by Prep-TLC (CH2Cl2/MeOH 20:1) to afford 256c (140 mg, 21.10%) as a yellow solid.

### 4. Synthesis of 256

256c (220 mg) was purified by Prep-Chiral-HPLC with the following conditions (Column: CHIRAL ART Cellulose-SB, 2*25 cm, 5 um; Mobile Phase A: Hex (0.5% 2M NH3-MeOH), Mobile Phase B: EtOH:DCM=1:1; Flow rate: 20 mL/min; Gradient: 15% B to 15% B in 11.3 min; Wave Length: 220/254 nm; RT1 (min): 8.02; RT2 (min): 11.04; first peak is product) to afford crude product (84 mg, 38.18%) as a yellow solid, Crude product (84 mg) was purified by Prep-Chiral-HPLC with the following conditions (Column: CHIRALPAK IF, 2*25 cm, 5 μm; Mobile Phase A: Hex (0.5% 2M NH3-MeOH), Mobile Phase B: EtOH; Flow rate: 20 mL/min; Gradient: 20% B to 20% B in 13 min; Wave Length: 220/254 nm; RT1 (min): 9.26; RT2 (min): 11.22; first peak is product) to afford 256 (17 mg, 20.24%) as a yellow solid.

LC-MS: (ES, m/z): [M+H]⁺ 607. H-NMR: (400 MHz, CD30D, δ ppm): 0.89-0.97 (m, 5H), 1.53-1.55 (m, 1H), 1.67-1.80 (m, 5H), 1.91-1.94 (m, 5H), 2.14-2.17 (m, 1H), 2.25-2.27 (m, 1H), 2.31-2.40 (m, 1H), 2.92-2.98 (m, 2H), 3.53 (s, 3H), 4.29-4.33 (m, 2H), 7.07 (s, 1H), 7.18 (s, 1H), 7.27-7.29 (d, 1H), 7.48-7.52 (t, 1H), 7.67-7.69 (m, 2H), 7.79 (s, 1H), 8.32 (s, 1H).

### Example 257 Synthesis of Compound 257

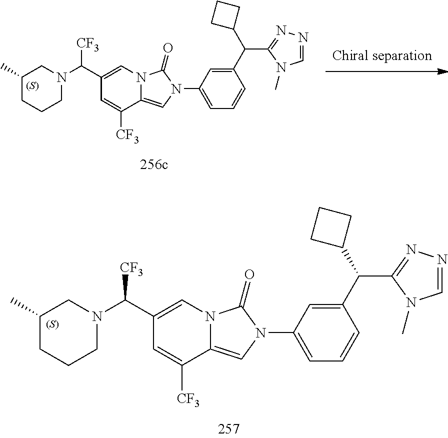

### Synthesis of 257

256c (220 mg) was purified by Prep-Chiral-HPLC with the following conditions (Column: CHIRAL ART Cellulose-SB, 2*25 cm, 5 μm; Mobile Phase A: Hex (0.5% 2M NH3-MeOH)-HPLC, Mobile Phase B: EtOH:DCM=1:1; Flow rate: 20 mL/min; Gradient: 15% B to 15% B in 113 min; Wave Length: 220/254 nm; RT1 (min): 8.02; RT2 (min): 11.04; first peak is product) to afford crude product (84 mg) as a yellow solid. Crude product (84 mg) was purified by Prep-Chiral-HPLC with the following conditions (Column: CHIRALPAK IF, 2*25 cm, 5 μm; Mobile Phase A: Hex (0.5% 2M N13-MeOH), Mobile Phase B: EtOH; Flow rate: 20 mL/min; Gradient: 20% B to 20% B in 13 min; Wave Length: 220/254 nm; RT1 (min): 9.26; RT2 (min): 11.22; second peak is product) to afford 257 (22 mg, 26.19%) as a yellow solid.

LC-MS: (ES, m/z): [M+H]−605. H-NMR: (400 MHz, CD30D, δ ppm): 0.89-0.97 (m, 5H), 1.61-1.78 (m, 6H), 1.88-1.93 (m, 5H), 2.06-2.08 (m, 1H), 2.25-2.27 (m, 1H), 2.34-2.43 (m, 1H), 2.89-2.9t (m, 2H), 3.50 (s, 3H), 4.28-4.33 (m, 2H), 7.07 (s, 1H), 7.18 (s, 1H), 7.27-7.29 (d, 1H), 7.48-7.52 (t, 1H), 7.65-7.68 (m, 2H), 7.79 (s, 1H), 8.32 (s, 1H).

### Example 258. Synthesis of Compound 258

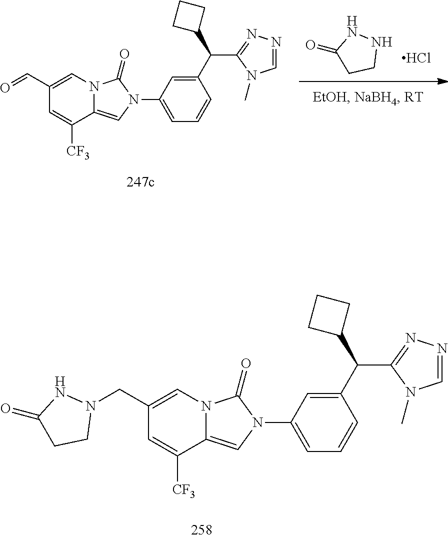

### Synthesis of 258

A solution of 247c (400 mg, 0,878 mmol, 1 equiv) and pyrazolidin-3-one hydrochloride (215.26 mg, 1.756 mmol, 2 equiv) in EtOH (6 mL) was stirred overnight at room temperature under nitrogen atmosphere. To the above mixture was added NaBH4 (49.84 mg, 1.317 mmol, 1.5 equiv) at room temperature. The resulting mixture was stirred for additional 1h at room temperature. The reaction was quenched with saturated N4Cl (aq.) (100 mL) at room temperature. The aqueous layer was extracted with DCM (3×20 mL). The organic layers were concentrated under reduced pressure. The residue was purified by Prep-TLC (DCM/MeOH 15:1) to afford the crude product. The crude product (30 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 16% B to 46% B in 8 min, Wave Length: 220 inn; RT1 (min): 7.73) to afford 258 (19.2 mg, 4.12%) as a yellow solid.

LC-MS: (ES, m/z): [M+H]+526. H-NMR: (400 MHz, DMSO-d6, δ ppm): 1.63-1.93 (m, 5H), 2.01-2.15 (m, 1H), 2.31-2.49 (m, 2H), 3.19-3.32 (m, 3H), 3.43 (s, 3H), 3.63 (s, 2H), 4.25-4.28 (d, 1H), 7.00 (s, 1H), 7.19-7.21 (d, 1H), 7.32 (s, 1H), 7.44-7.46 (m, 1H), 7.71-7.75 (m, 3H), 8.33 (s, 1H), 9.36 (s, 1H).

### Example 259. Synthesis of Compound 259

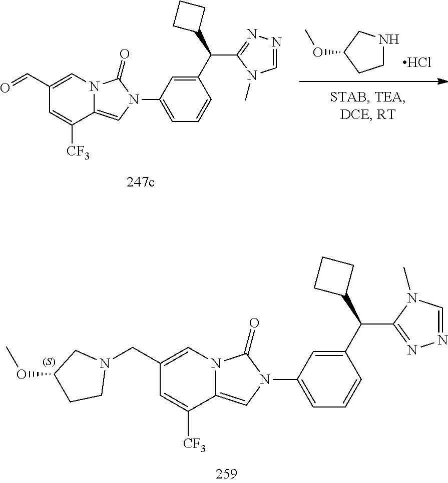

### Synthesis of 259

To a stirred solution of 247c (250 mg, 0,549 mmol, 1 equiv) and (3S)-3-methoxypyrrolidine hydrochloride (111.05 mg, 1.098 mmol, 2 equiv) in DCE (3 mL) was added TEA (111.09 mg, 1.098 mmol, 2 equiv) in DCE (3 mL) at room temperature. The mixture was stirred for 30 min at room temperature. To the above mixture was added STAB (232.68 mg 1.098 mmol, 2 equiv) at room temperature. The resulting mixture was stirred for overnight at room temperature. The reaction was quenched by the addition of sat. NH4Cl (aq.) (5 mL) at room temperature. The resulting mixture was extracted with EtOAc (3×10 mL). The resulting mixture was concentrated under reduced pressure. The crude product (89 mg) was purified by Prep-HPLC with the following conditions (Column: Kinetex EVO prep C18, 30*150, 51m; Mobile Phase A: Water (10 mmol/L NH4HCO3), Mobile Phase B: ACN; Flow rate: 60 mL/min; Gradient: 30% B to 40% B in 11 min, 40% B; Wave Length: 220 m; RT1 (min): 10.25) to afford 259 (66.6 mg, 21.26%) as a yellow solid.

LC-MS: (ES, m/z): [M+H]+: 541. H-NMR: (400 MHz, DMSO-d6, ppm): δ 1.60-1.72 (m, 2H), δ 1.73-1.85 (m, 4H), δ 1.93-2.15 (m, 2H), δ 2.51-2.53 (d, 2H), δ 2.52 (s, 1H), δ 2.54-2.57 (m, 1H), δ 2.71-2.73 (d, 1H), δ 3.16 (s, 3H), δ 3.21-3.23 (d, 1H), δ 3.35 (s, 1H), δ 3.52 (s, 2H), δ 3.58 (s, 3H), δ 3.85-3.87 (d, 1H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 2H), δ 7.72 (s, 1H), δ 8.33 (s, 1H).

### Example 260. Synthesis of Compound 260

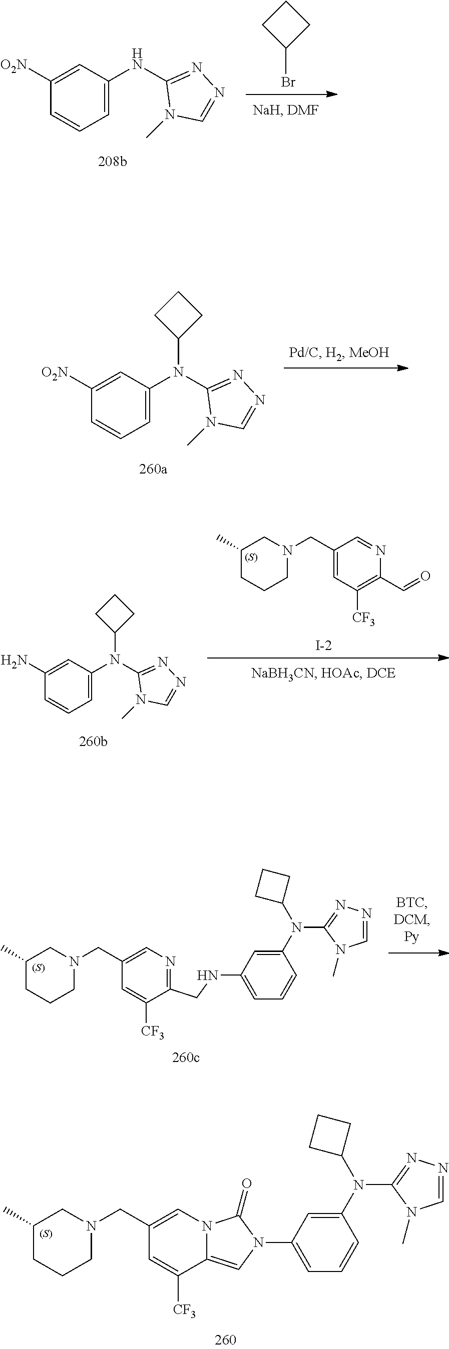

### 1. Synthesis of 260a

Into a 100m 3-necked round-bottom flask were added 208b (700 mg, 1,597 mmol, 1 equiv) in DMF (8 mL) and NaH (191.58 mg, 4.791 mmol, 3 equiv) at 0° C. The resulting mixture was stirred for 1h at 0° C. under nitrogen atmosphere. To the above mixture was added bromocyclobutane (646.68 mg, 4.791 mmol, 3 equiv). The resulting mixture was stirred for additional overnight at 80° C. The reaction was quenched by the addition of NH4Cl (aq.) (100 mL) at room temperature. The aqueous layer was extracted with EtOAc (3×30 mL). The resulting mixture was concentrated under reduced pressure. The residue was purified by Prep-TLC (CH2Cl2/MeOH 10:1) to afford 260a (250 mg, 28.65%) as white solid.

### 2. Synthesis of 260b

To a solution of 260a (240 mg, 0.878 mmol, 1 equiv) in MeOH (6 mL) was added Pd/C (50 mg, 10%) under nitrogen atmosphere in a 100 mL round-bottom flask. The mixture was hydrogenated at room temperature for overnight under hydrogen atmosphere by using a hydrogen balloon, filtered through a Celite pad and concentrated under reduced pressure. The residue was purified by Prep-TLC (CH2Cl2/MeOH 10:1) to afford 260b (210 mg, 98.28%) as white solid.

### 3. Synthesis of 260c

Into a 8 mL sealed tube were added 260b (200 mg, 0.822 mmol, 1 equiv) and I-2 (282.40 mg 0.986 mmol, 1.2 equiv) in DCE (3 ml) at room temperature. The resulting mixture was stirred for 30 min at room temperature under nitrogen atmosphere. To the above mixture was added NaBH(OAc)3 (261.72 mg, 1.233 mmol, 1.5 equiv) and HOAc (49.36 mg, 0.822 mol, 1 equiv). The resulting mixture was stirred for additional overnight at room temperature. The reaction was quenched by the addition of water (50 mL) at room temperature. The aqueous layer was extracted with EtOAc (4×20 mL). The resulting mixture was concentrated under reduced pressure. The residue was purified by Prep-TLC (CH2Cl2/MeOH 10:1) to afford 260c (160 mg, 37.90%) as yellow oil.

### 4. Synthesis of 260

To a stirred mixture 260c (150 mg, 0.292 mmol, 1 equiv) and Pyridine (138.61 mg, 1.752 mmol, 6 equiv) in DCM (6 mL) was added triphosgene (30.33 mg, 0.102 mmol, 0.35 equiv) at room temperature. The resulting mixture was stirred for 30 min at room temperature. The reaction was quenched with NaHCO₃ (aq.) (20 mL) at room temperature. The aqueous layer was extracted with EtOAc (3×15 mL). The resulting mixture was concentrated under reduced pressure. The residue was purified by Prep-TLC (CH2Cl2/MeOH 10:1) to afford 260 (60.6 mg, 38.45%) as a yellow solid

LC-MS: (ES, m/z): [M+H]+540. H-NMR: (400 MHz, DMSO, δ ppm): 0.77-0.91 (m, 4H), 1.38-1.51 (m, 1H), 1.55-1.73 (m, 6H), 1.77-1.91 (m, 3H), 2.27-2.29 (m, 2H), 2.72-7.74 (m, 2H), 3.24 (s, 2H), 3.40 (s, 3H), 4.47-4.51 (t, 1H), 6.44-6.47 (m, 1H), 6.99 (s, 1H), 7.19-7.20 (m, 2H), 7.27 (s, 1H), 7.31-7.35 (m, 1H), 7.65 (s, 1H), 8.58 (s, 1H).

### Example 261. Synthesis of Compound 261

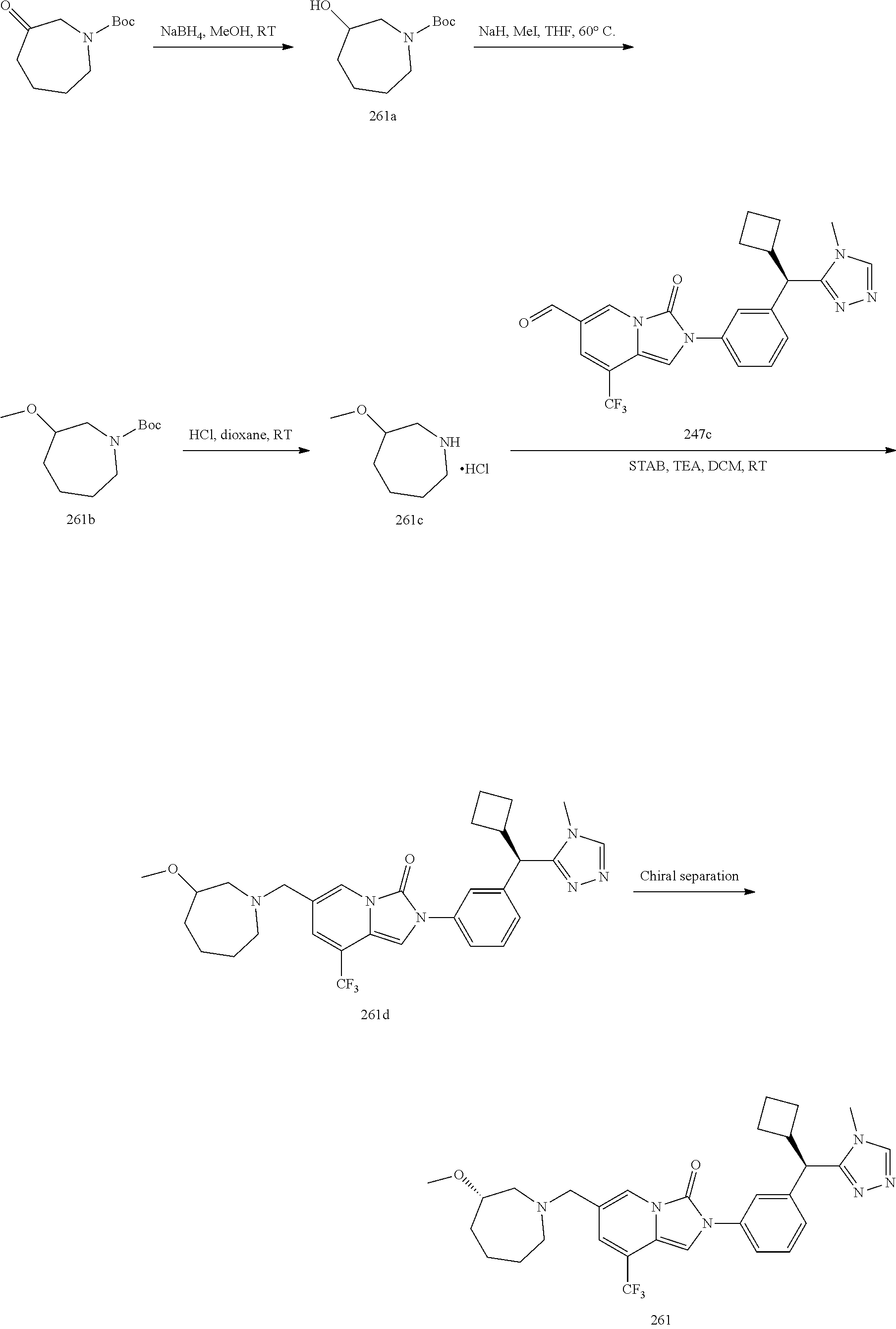

### 1. Synthesis of 261a

Into a 250-mL 3-necked round-bottom flask purged and maintained with an inert atmosphere of nitrogen, was placed tert-butyl 3-oxoazepane-1-carboxylate (5.2 g, 24.381 mmol, 1 equiv), MeOH (60 mL), NaBH4 (1844.70 mg, 48,762 mmol, 2 equiv). The resulting solution was stirred for 2 hr at 25 degrees C. The reaction was then quenched by the addition of 100 ml-of water. The resulting solution was extracted with 3×100 mL of ethyl acetate and concentrated under vacuum. This resulted in 261a (5.2 g, 88.17%) as a brown solid.

### 2. Synthesis 261b

Into a 100-mL 3-necked round-bottom flask purged and maintained with an inert atmosphere of nitrogen, was placed 261a (2.8 g, 13.006 mmol, 1 equiv), THY (30 mL), NaH (936.32 mg, 39,018 mmol, 3 equiv). The resulting solution was stirred for 1 hr at room temperature. To the above mixture was added CH3I (5537.98 mg, 39,018 mmol, 3 equiv). The resulting solution was stirred for 2 hr at 60 degrees C. The reaction was then quenched by the addition of 100 mL of NH4Cl. The resulting solution was extracted with 3×100 mL of ethyl acetate and concentrated under vacuum. This resulted in 261b (2.5 g, 77.12%) as a white solid.

### 3. Synthesis of 261c

Into a 250-mL 3-necked round-bottom flask was placed 261b (2.5 g, 10.902 mmol, 1 equiv), dioxane (30 mL), HCl in dioxane (3 mL). The resulting solution was stirred for 2 hr at 25 degrees C. The reaction solution was concentrated under vacuum. This resulted in 261c (3 g, crude) as a yellow oil.

### 4. Synthesis of 261d

To a stirred mixture of 261c (436.48 mg, 2.634 mmol, 2 equiv) and 247c (600 mg, 1,317 mmol, 1.00 equiv) in DCE (6 mL) was added Et3N (399.94 mg, 3.951 mmol, 3 equiv) at room temperature. The resulting mixture was stirred for 30 min at room temperature. To the above mixture was added NaBH(OAc)3 (837.63 mg, 3.951 mmol, 3 equiv) at room temperature. The resulting mixture was stirred for additional 4 h at room temperature. The reaction was quenched by the addition of water (30 mL) at room temperature. The aqueous layer was extracted with EtOAc (2×15 mL). The resulting mixture was concentrated under reduced pressure. The residue was purified by TLC with CH2Cl2/MeOH=15:1 to afford the crude product (300 mg) as a yellow solid. The crude product (300 mg) was purified by Prep-HPLC with the following conditions (Column: XBridge Prep OBD C18 Column, 30*150 mm, 5 μm; Mobile Phase A: water (10 mmol/L NH4HCO3), Mobile Phase B: ACN; Flow rate: 60 mL/min; Gradient: 41% B to 66% B in 8 min, 66% B; Wave Length: 220 nm; RT1 (min): 7.58) to afford 261d (120 mg, 15.38%) as a yellow solid.

### 5. Synthesis of 261

261d (120 mg, 0.211 mmol, 1 equiv) was separated by Prep-HPLC AL-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1. Flow rate: 20 mL/min; Gradient: 60% B to 60% B in 11 min; Wave Length: 220/254 nm; RT1 (min): 6.78; RT2 (min): 9.06; the first peak was product) to afford 261 (43.2 mg, 35.42%) as a yellow solid.

LCMS: (ES, m/z): [M+H]⁺ 569. H-NMR: (400 MHz, DMSO-d6, ppm, δ): 1.38-1.52 (m, 1H), 1.52-1.65 (m, 4H), 1.65-1.84 (m, 6H), 2.05-2.15 (m, 1H), 2.62-2.72 (m, 3H), 2.80-2.88 (m, 1H), 3.14 (s, 3H), 3.14-3.29 (m, 1H), 3.29-3.30 (m, 1H), 3.43 (s, 3H), 3.43-3.47 (m, 2H), 4.25-4.27 (d, 1H), 7.08 (s, 1H), 7.18-7.20 (d, 1H), 7.30 (s, 1H), 7.42-7.46 (t, 1H), 7.68-7.74 (m, 3H), 8.32 (s, 1H).

### Example 262. Synthesis of Compound 262

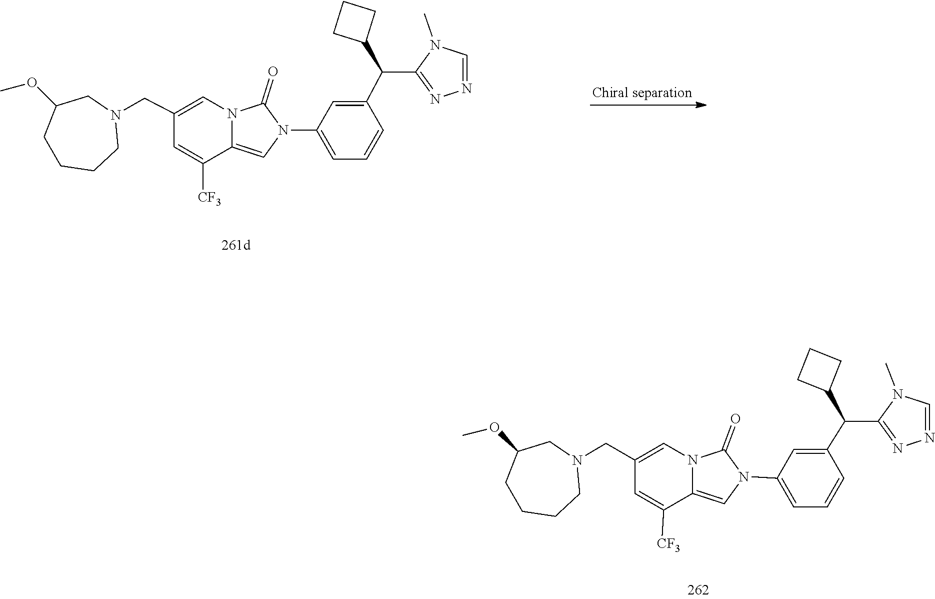 [SMILES: COC1CCCCN(Cc2cc(C(F)(F)F)c3cn(-c4cccc([C@H](c5nncn5C)C5CCC5)c4)c(=O)n3c2)C1 ; CO[C@@H]1CCCCN(Cc2cc(C(F)(F)F)c3cn(-c4cccc([C@H](c5nncn5C)C5CCC5)c4)c(=O)n3c2)C1]

### Synthesis of 262

261d (120 mg) was separated by Prep-CHIRAL-HPLC with the following conditions (Column: CHIRALPAK IC, 2*25 cm, 5 μm; Mobile Phase A: Hex (0.5% 2M NH3-MeOH), Mobile Phase B: EtOH:DCM=1l: 1; Flow rate: 20 mL/min; Gradient: 60% B to 60% B in 11 min; Wave Length: 220/254 nm; RT1 (min): 6.78; RT2 (min): 9.06; the second peak was product) to afford 262 (43.6 mg, 36.22%) as a yellow solid.

LCMS: (ES, m/z): [M+H]⁺ 569. H-NMR: (400 MHz, DMSO-d6, ppm, δ): 1.38-1.52 (m, 1H), 1.52-1.65 (m, 4H), 1.65-1.88 (m, 6H), 2.08-2.12 (m, 1H), 2.52-2.72 (m, 3H), 2.78-2.79 (m, 1H), 3.14 (s, 3H), 3.14-3.28 (m, 2H) 3.43-3.47 (m, 5H), 4.25-4.27 (d, 1H), 7.07 (s, 1H), 7.18-7.20 (d, 1H), 7.30 (d, 1H), 7.42-7.46 (t, 1H), 7.69-7.75 (m, 3H), 8.33 (s, 1H).

### Example 263. Synthesis of Compound 263

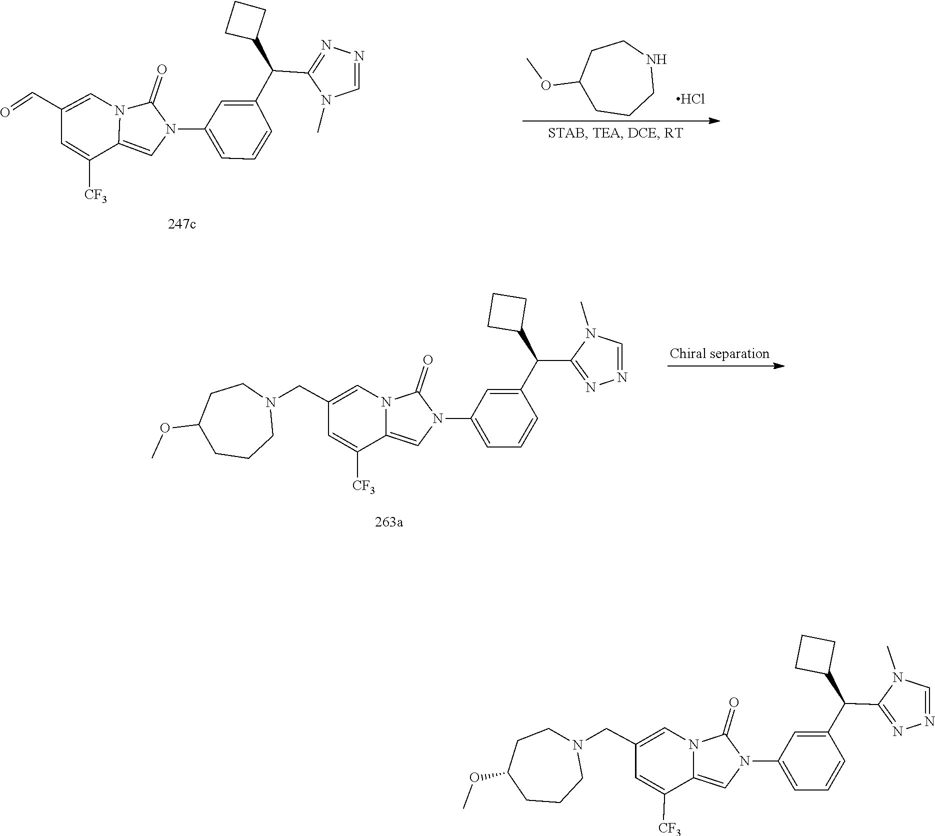

### 1. Synthesis of 263a

To a stirred mixture of 4-methoxyazepane hydrochloride (436.48 mg, 2.634 mmol, 2 equiv) and 247c (600 mg, 1.317 mmol, 1.00 equiv) in DCE (6 m L) was added Et3N (399.94 mg, 3.951 mmol, 3 equiv) at room temperature. The resulting mixture was stirred for 30 min at room temperature. To the above mixture was added NaBH(OAc)3 (837.63 mg, 3,951 mmol, 3 equiv) at room temperature. The resulting mixture was stirred for additional 4 h at room temperature. The reaction was quenched by the addition of water (30 mL) at room temperature. The aqueous layer was extracted with EtOAc (2×15 mL). The resulting mixture was concentrated under reduced pressure. The residue was purified by silica gel column chromatography, eluted with CH2Cl2/MeOH=15:1 to afford the crude product (400 mg) as a yellow solid. The crude product (400 mg) was purified by Prep-4-HPLC with the following conditions (Column: X-Bridge Prep OBD C18 Column, 30*150 mm, 5 μm; Mobile Phase A: water (10 mmol/L NH4HCO3), Mobile Phase B: ACN; Flow rate: 60 mL/min; Gradient: 35% B to 60% B in 7 min, 60% B; Wave Length: 220 nm; RT1 (min): 7.60) to afford 263a (170 mg, 21.79%) as a yellow solid.

### 2. Synthesis of 263

263a (170 mg) was separated by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 60% B to 60% B in 16.5 min; Wave Length: 220/254 nm; RT1 (min): 8.20; RT2 (min): 13.40; the first peak was product) to afford 263 (33.5 mg, 19.47%) as a yellow solid.

LCMS (ES, m/z): [M+H]⁺ 569. H-NMR: (400 MHz, DMSO-d6, ppm, δ): 1.38-1.52 (m, 1H), 1.52-1.90 (m, 1H), 2.04-2.15 (m, 1H), 2.55-2.72 (m, 3H), 3.19-3.27 (m, 4H), 3.39-3.43 (m, 6H), 4.25-4.27 (d, 1H), 7.03 (s, 1H), 7.20-7.20 (d, 1H), 7.30 (s, 1H), 7.44-7.46 (t, 1H), 7.67-7.74 (m, 3H), 8.32 (s, 1H).

### Example 264. Synthesis of Compound 264

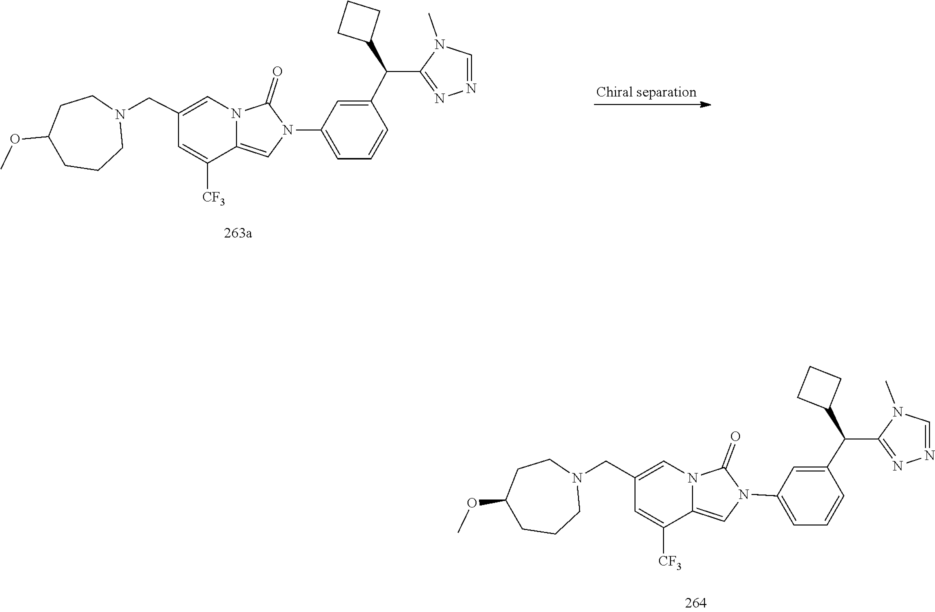 [SMILES: COC1CCCN(Cc2cc(C(F)(F)F)c3cn(-c4cccc([C@H](c5nncn5C)C5CCC5)c4)c(=O)n3c2)CC1 ; CO[C@@H]1CCCN(Cc2cc(C(F)(F)F)c3cn(-c4cccc([C@H](c5nncn5C)C5CCC5)c4)c(=O)n3c2)CC1]

### 1. Synthesis of 264

263a (170 mg) was separated by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 60% B to 60% B in 16.5 min; Wave Length: 220/254 nm; RT1 (min): 8.20; RT2 (min): 13.40; the second peak is product) to afford 264 (33.3 mg, 19.45%) as a yellow solid.

LCMS: (ES, m/z): [M+H]⁺ 569. H-NMR: (400 MHz, DMSO-d6, ppm, δ): 1.38-1.52 (m, 1H), 1.52-1.87 (m, 1H), 2.04-2.15 (m, 1H), 2.55-2.72 (m, 3H), 3.19-3.27 (m, 4H), 3.39-3.43 (m, 6H), 4.25-4.27 (d, 1H), 7.03 (s, 1H), 7.20-7.20 (d, 1H), 7.30 (s, 1H), 7.44-7.46 (t, 1H), 7.67-7.74 (m, 3H), 8.32 (s, 1H).

### Example 265. Synthesis of Compound 265

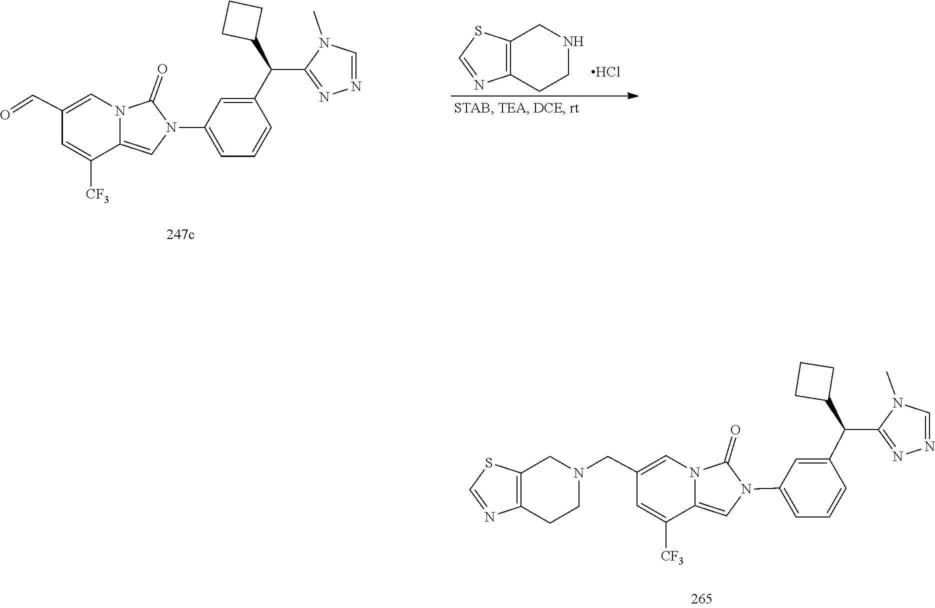 [SMILES: Cn1cnnc1[C@@H](c1cccc(-n2cc3c(C(F)(F)F)cc(C=O)cn3c2=O)c1)C1CCC1 ; c1nc2c(s1)CNCC2 ; Cl ; Cn1cnnc1[C@@H](c1cccc(-n2cc3c(C(F)(F)F)cc(CN4CCc5ncsc5C4)cn3c2=O)c1)C1CCC1]

### Synthesis of 265

Into a 50 mL 3-necked round-bottom flask were added 4H,5H,6H,7H-[1,3]thiazolo[5,4-c]pyridine hydrochloride (116.37 mg, 0,659 mmol, 1 equiv), 247c (300 mg, 0.659 mmol, 1.00 equiv), TEA (199.97 mg, 1.977 mmol, 3 equiv) and DCE (3 mL) at room temperature. The resulting mixture was stirred for 1h at room temperature. To the above mixture was added STAB (418.82 mg, 1,977 mmol, 3 equiv) at room temperature. The resulting mixture was stirred for additional overnight at room temperature. The reaction was quenched with water (10 mL) at room temperature. The aqueous layer was extracted with EtOAc (3×15 mL). The resulting mixture was concentrated under vacuum. The crude product was purified by Prep-HPLC with the following conditions (Column: XBridge Shield RP18 OBD Column, 30*150 mm, 5 nm; Mobile Phase A: Water (10 mmol/T NH4HCO3+0.1% NH3·H2O), Mobile Phase B: ACN; Flow rate: 60 mL/min; Gradient: 31% B to 51% B in 8 min, 51% B; Wave length: 220 nm; RT1 (min): 7.73) to afford 265.

H-NMR: (400 MHz, DMSO-d6, ppm, δ): δ 1.62-1.86 (m, 5H), δ 2.11-2.12 (d, 1H), δ 2.82-2.88 (m, 4H), δ 3.18-3.25 (m, 1H), δ 3.43 (s, 3H), δ 3.53 (s, 2H), δ 3.77 (s, 2H), δ 4.25-4.28 (d, 1H), δ 7.07 (s, 1H), δ 7.19-7.21 (d, 1H), δ 7.32 (s, 1H), δ 7.42-7.46 (t, 1H), δ 7.69-7.75 (m, 3H), δ 8.33 (s, 1H), δ 8.91 (s, 1H).

### Example 266. Synthesis of Compound 266

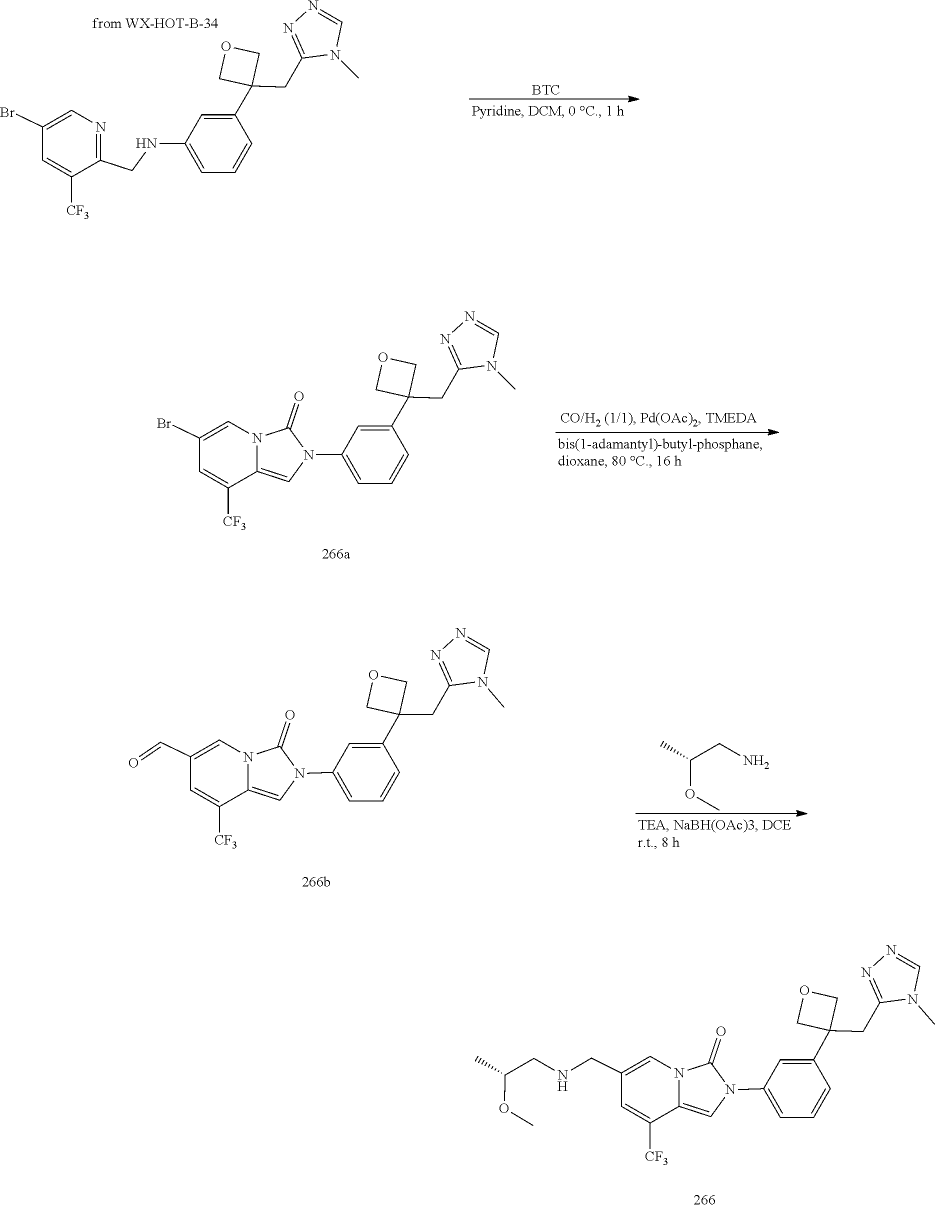

### 1. Synthesis of 266a

To a solution of N-[[5-bromo-3-(trifluoromethyl)-2-pyridyl]methyl]-3-[3-[(4-methyl-1,2,4-triazol-3-yl)methyl]oxetan-3-yl]aniline (7 g, 14.51 mmol, 1 eq) in DCM (60 mL) was added Py (5.74 g, 72.57 mmol, 5.86 mL, 5 eq) at 0° C. Then a solution of TRIPHOSGENE (4.98 g, 16.78 mmol, 1.16 eq) in DCM (10 mL) was slowly added under N2. The mixture was stirred at 0° C. for 1 hr. The reaction mixture was quenched by addition of a.q. NaHCO₃50 mL at 0° C., then extracted with DCM 300 mL. (100 ml.*3). The combined organic layers were dried over Na2SO4, filtered and concentrated under reduced pressure to give a residue. The residue was purified by column chromatography (SiO2, DCM/MeOH=50/1 to 0/1) to give 266a (2.8 g, 5.51 mmol, 37% yield) as a red solid,

### 2. Synthesis of 266b

To a solution of 266a (1.8 g, 3.54 mmol, 1 eq) in dioxane (72 mL) was added Pd(OAc)2 (159.01 mg, 708.26 umol, 0.2 eq), TMEDA (823.06 mag, 7.08 mmol, 1.07 mL, 2 eq) and bis(1-adamantyl)-butyl-phosphane (253.94 mg, 708.26 umol, 0.2 eq). The mixture was stirred at 80° C. for 16 hr under CO/H2 (v/v=1/1). The reaction was then cooled to room temperature and poured into water (250 mL), extracted with EtOAc (3*50 mL). The combined organic layers were combined and then concentrated. The residue was purified by column chromatography (Al2O3, DCM/MeOH=100/1 to 20/1) to give 266b (0.86 g, 1.88 mmol, 53% yield) as a yellow solid.

### 3. Synthesis of 266

To a solution of (2R)-2-methoxypropan-1-amine (82.38 mg, 655.87 umol, HC, 3 equiv) and 266b (0.1 g, 218.62 umol, 1 equiv) in DCE (0.5 mL) was added TEA (88.49 mg, 874.50 umol, 121.72 uL, 4 equiv). The mixture was stirred at 20° C. for 1 hr. Then it was added NaBH(OAc)3 (92.67 mg, 437.25 umol, 2 equiv) under N2. The mixture was stirred at 20° C. for 1 hr. The reaction mixture was quenched by the addition of water (10 mL) and extracted with EtOAc (2*20 ml). The organic phase was dried over anhydrous sodium sulfate, filtered and the filtrate was concentrated under reduced pressure to give a residue. The residue was purified by prep-HPLC (column: Phenomenex Luna C18 75*30 mm*3 um; mobile phase: [water (F A)-ACN]; B %: 20%-50%, 8 min) to afford 266 (20 mg, 37.70 umol, 17.2% yield) as a yellow solid.

MS: (ES, m/z): [M+H]⁺ 531.2. H-NMR: (400 MHz, DMSO-d6) δ 8.31 (s, 1H), 8.16 (s, 1 H), 7.75 (s, 1H), 7.62 (d, J=8.4 Hz, 1H), 7.40 (t, J=7.8 Hz, 1H), 7.28 (s, 1H), 7.22 (s, 1 H), 7.14 (s, 1H), 6.90 (d, J=8 Hz, 1H), 4.94 (d, J=6 Hz, 2 H), 4.88 (d, J=5.2 Hz, 2 H), 3.67 (s, 2H), 3.51 (s, 2H), 3.46-3.43 (m, 1H), 3.22 (s, 3H), 2.91 (s, 3H), 2.62-2.66 (m, 2 H), 1.05 (d, J=6. Hz, 3H).

### Example 267. Synthesis of Compound 267

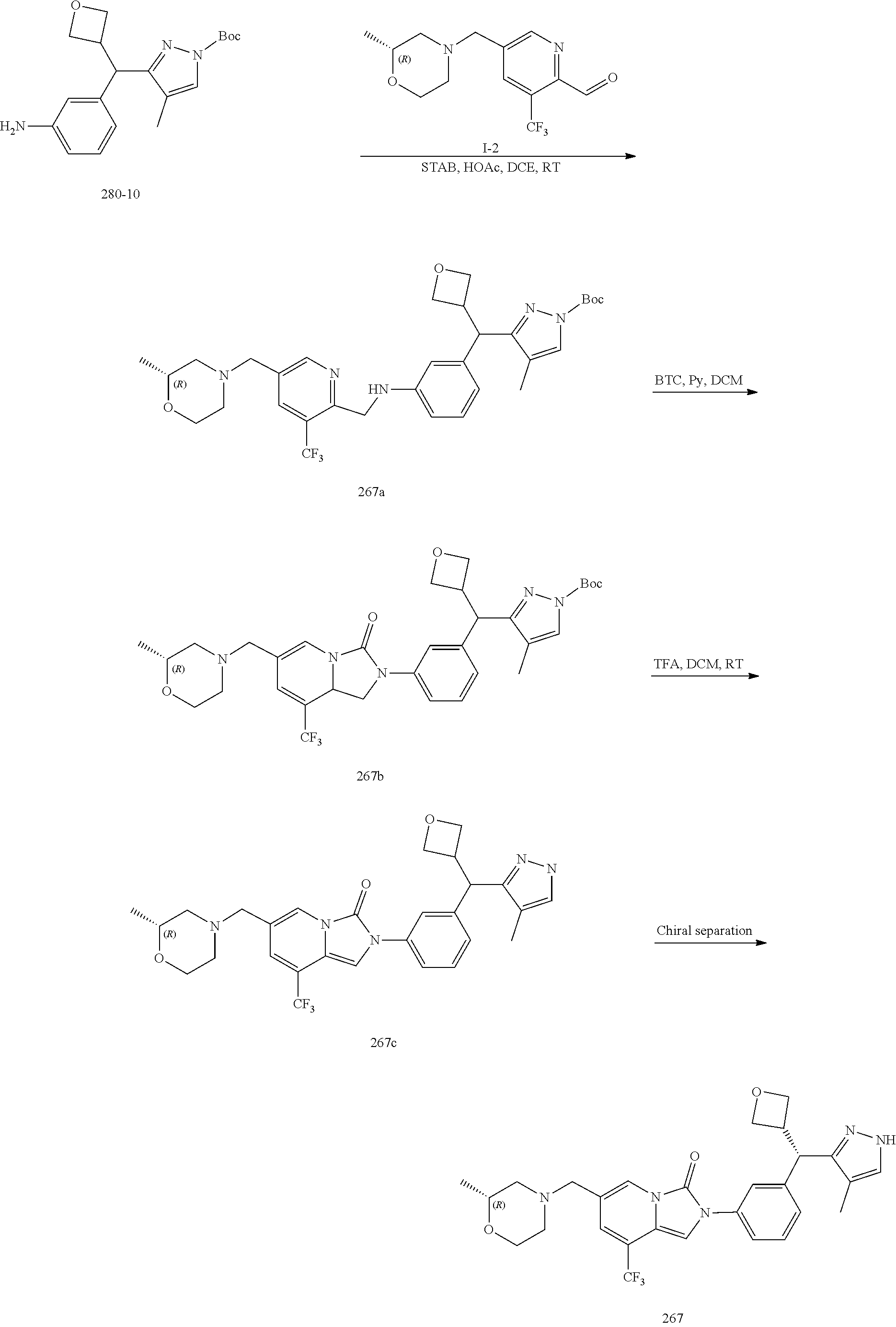

### 1. Synthesis of 267a

To a stirred solution of 280-10 (300 mg, 0.874 mmol, 1 equiv) and I-2 (327.36 mg, 1.136 mmol, 1.3 equiv) in DCE (5 mL) was added STAB (370.28 mg, 1.748 mmol, 2 equiv) at room temperature under nitrogen atmosphere. The resulting mixture was stirred for overnight at room temperature. The reaction was quenched with saturated NaHCO₃ (aq.) (100 mL) at room temperature. The aqueous layer was extracted with DCM (2×50 mL). The organic layers were concentrated under reduced pressure. The residue was purified by Prep-TLC (DCM/MeOH 30:1) to afford 267a (300 mg, 51.87%) as a white solid.

### 2. Synthesis of 267b

To a stirred solution of 267a (280 mg, 0,455 mmol, 1 equiv) and Pyridine (359.72 mg, 4.550 mmol, 10 equiv) in DCM (10 mL) was added Triphosgene (67.47 mg, 0.228 mmol, 0.5 equiv) at room temperature under nitrogen atmosphere. The resulting mixture was stirred for 10 min at room temperature. The reaction was quenched with saturated NaHCO3 (aq.) (100 mL) at room temperature. The aqueous layer was extracted with DCM (2×20 mL). The organic layers were concentrated under reduced pressure. The crude product was used in the next step directly without further purification. This resulted in 267b (200 mg, 61.68%) as a yellow solid.

### 3. Synthesis of 267c

To a stirred solution of 267b (190 mg, 0,296 mmol, 1 equiv) in DCM (6 mL) was added TFA (2 mL) at room temperature. The resulting mixture was stirred for 1h at room temperature. The resulting mixture was concentrated under vacuum. The crude product (200 mg) was purified by Prep-HPLC with the following conditions (Column: XBridge Shield RP18 OBD Column, 30*150 mm, 5 μm; Mobile Phase A: Water (10 mmol/L NH4HCO3+0.1% NH3H2O), Mobile Phase B: ACN; Flow rate: 60 mL/min; Gradient: 31% B to 51% B in 8 min, Wave Length: 220 nm; RT (min): 7.78) to afford 267c (110 mg, 67.23%) as a yellow solid.

### 4. Synthesis of 267

267c (110 mg) was purified by Prep-Chiral-HPLC with the following conditions (Column: CHIRALPAK OD-H, 2*25 cm, 5 μm; Mobile Phase A: Hex (0.5% 2M NH3-MeOH), Mobile Phase B: EtOH; Flow rate: 20 mL/min; Gradient: 20% B to 20% B in 22 min; Wave Length: 220/254 nm; RT1 (min): 13.69; RT2 (min): 19.09; the first peak is product) to afford 267 (28.4 mg, 25.64%) as a yellow solid.

LC-MS-267: (ES, m/z): [M+H]⁺ 542. H-NMR-267 (400 MHz, CD30D, δ ppm): 1.14-1.15 (d, 3H), 1.76 (s, 3H), 1.91-2.01 (m, 1H), 2.20-2.31 (m, 1H), 2.78-2.87 (m, 2H), 3.05-3.15 (m, 1H), 3.40 (s, 2H), 3.65-3.71 (m, 2H), 3.79-3.81 (m, 2H), 3.86-3.89 (m, 1H), 4.02-4.06 (m, 1H), 4.32-4.34 (d, 1H), 4.38-4.42 (m, 1H), 7.11 (s, 1H), 7.16 (s, 1H), 7.25-7.27 (d, 1H), 7.34 (s, 1H), 7.52-7.56 (m, 1H), 7.64-7.66 (m, 2H), 7.71 (s, 1H).

### Example 268. Synthesis of Compound 268

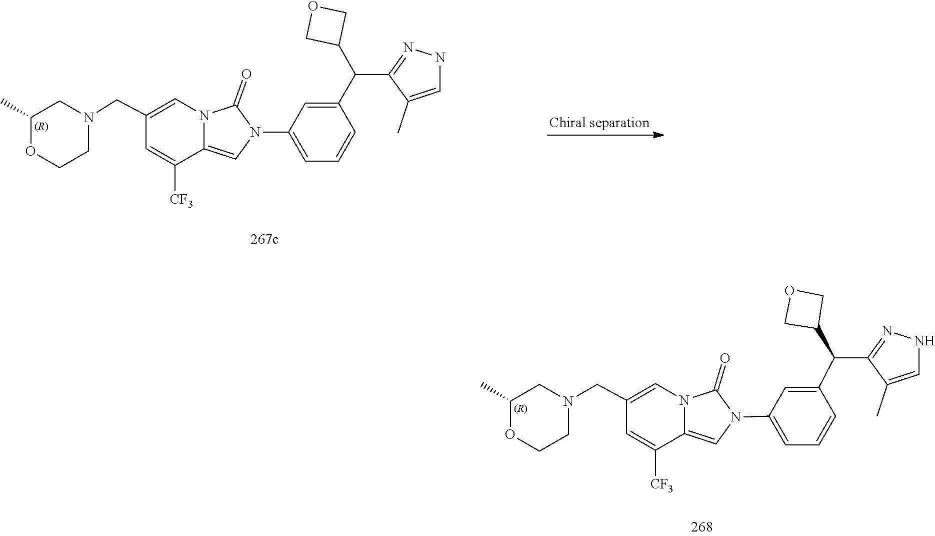

### Synthesis of 268

267c (110 mg) was purified by Prep-Chiral-HPLC with the following conditions (Column: CHIRALPAK OD-H, 2*25 cm, 5 μm; Mobile Phase A: Hex (0.5% 2M NH3-MeOH), Mobile Phase B: EtOH; Flow rate: 20 mL/min; Gradient: 20% B to 20% B in 22 min; Wave Length: 220/254 nm; RT1 (min): 13.69; RT2 (min): 19.09; the second peak is product) to afford 268 (26.6 mg, 24.01%) as a yellow solid.

LC-MS: (ES, m/z): [M+H]⁺ 542. H-NMR: (400 MHz, CD30D, δ ppm): 1.15-1.17 (d, 3H), 1.76 (s, 3H), 1.94-2.08 (m, 1H), 2.24-2.31 (m, 1H), 2.85-2.93 (m, 2H), 3.06-3.11 (m, 1H), 3.47 (s, 2H), 3.67-3.73 (m, 2H), 3.79-3.81 (m, 2H), 3.88-3.91 (m, 1H), 4.02-4.06 (m, 1H), 4.33-4.34 (d, 1H), 4.38-4.42 (m, 1H), 7.11 (s, 1H), 7.17 (s, 1H), 7.25-7.27 (d, 1H), 7.35 (s, 1H), 7.52-7.56 (m, 1H), 7.64-7.66 (m, 2H), 7.74 (s, 1H).

### Example 269. Synthesis of Compound 269

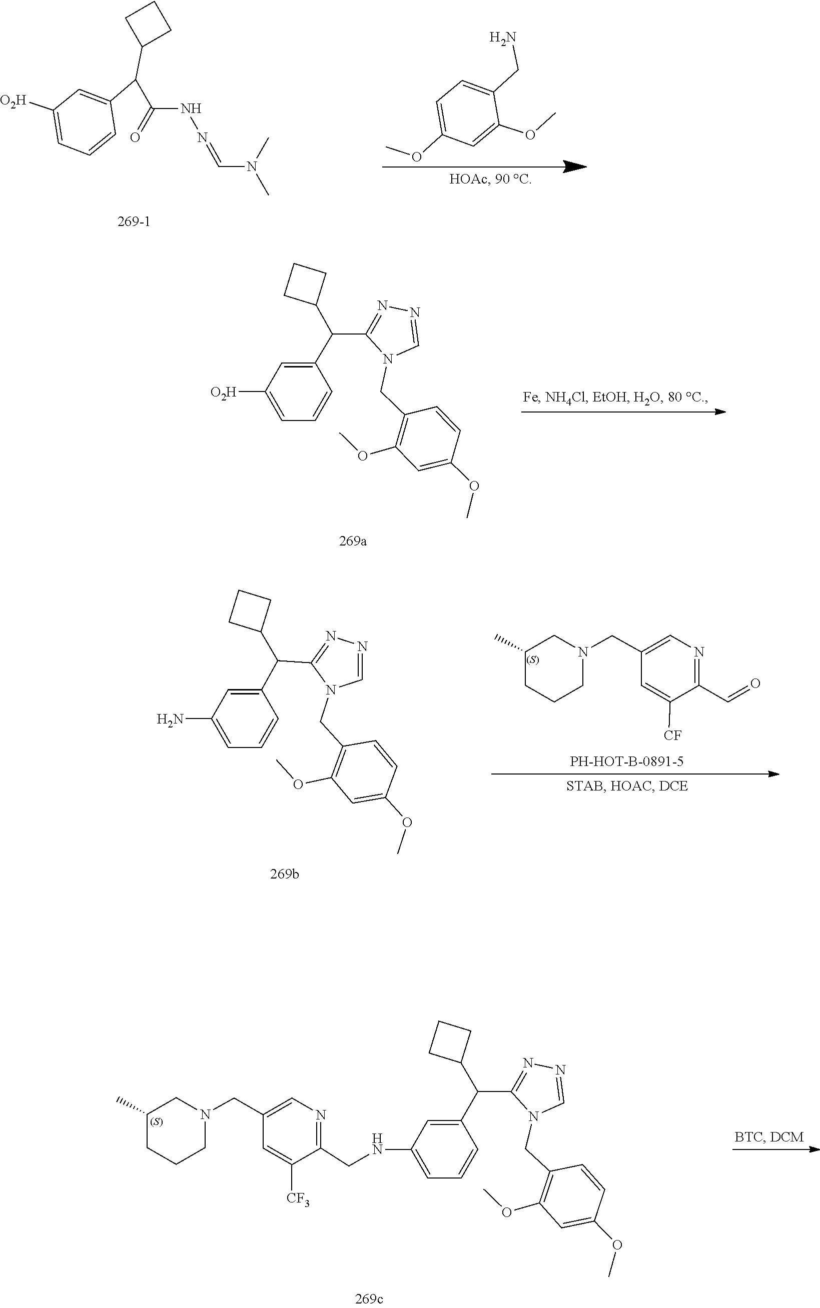

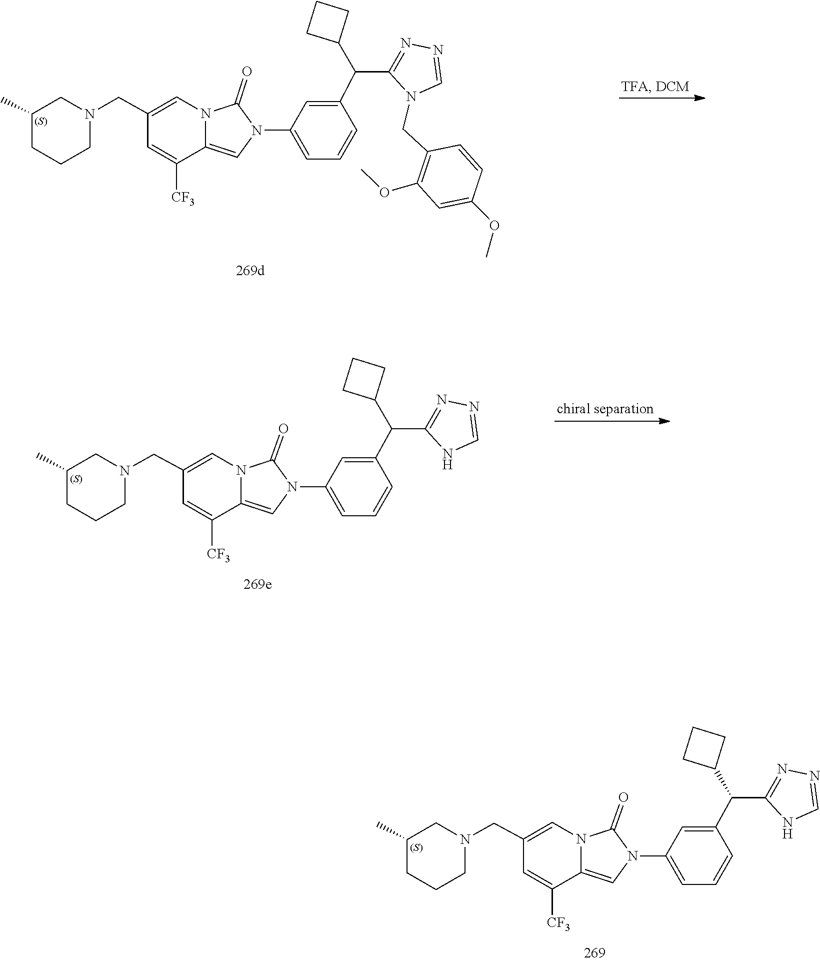

### 1. Synthesis of 269a

A mixture of 261-1 (3 g, 9.857 mmol, 1 equiv) and 1-(2,4-dimethoxyphenyl)methanamine (4.12 g, 24,642 mmol, 2.5 equiv) in HOAc (30 mL) was stirred for overnight at 90° C. The mixture was basified to pH 7 with saturated NaHCO₃ (aq.). The aqueous layer was extracted with EtOAc (3×100 mL). The resulting mixture was concentrated under reduced pressure. The residue was purified by reverse flash chromatography with the following conditions: C18 silica gel 330 g; mobile phase, MeCN in 0.1% NH4HCO3 water, 20% to 50% gradient in 15 min; detector, UV 254 nm to afford PH-BOT-B-1302-1 (560 mg, 13.35%) as a colorless oil,

### 2. Synthesis of 269b

To a stirred mixture of 269a (560 mg, 1.420 mmol, 1 equiv) and Fe (237.86 mg, 4.260 mmol, 3 equiv) in EtOH (6 mL) was added NH4Cl (379.72 mg, 7.100 mmol, 5 equiv) in 1120 (1.5 mL) at room temperature. The resulting mixture was stirred for overnight at 80° C. under nitrogen atmosphere. The reaction was quenched with Water at room temperature. The aqueous layer was extracted with EA (3×10 mL). The resulting mixture was concentrated under reduced pressure. The residue was purified by Prep-TLC (PE/EA 5:1) to afford 269b (290 mg, 51.81%) as a yellow solid.

### 3. Synthesis of 269c

Into a 100 mL round-bottom flask were added 269b (290 mg, 0.766 mmol, 1 equiv), DCE (5 mL), 1-2 (219.37 mg, 0,766 mmol, 1 equiv), HOAC (46.01 mg, 0.766 mmol, 1 equiv) and STAB (324.79 mg, 1.532 mmol, 2 equiv) at room temperature. The resulting mixture was stirred for overnight at room temperature. The reaction was quenched by the addition of sat. NH4Cl (aq.) (20 mL) at room temperature. The aqueous layer was extracted with EtOAc (3×10 mL). The resulting mixture was concentrated under reduced pressure. The residue was purified by Prep-TLC eluted with CH2Cl2/MeOH (12:1) to afford 269c (200 mg, 40.23%) as a light yellow solid.

### 4. Synthesis of 269d

Into a 100 mL round-bottom flask were added 269c (200 mg, 0.308 mmol, 1 equiv), DCE (5 mL), Pyridine (146.31 mg, 1.848 mmol, 6 equiv) and Triphosgene (32.02 mg 0.108 mmol, 0.35 equiv) at room temperature. The resulting mixture was stirred for 5 min at room temperature under nitrogen atmosphere. The reaction was quenched by the addition of sat, NaHCO₃ (aq.) (20 mL) at room temperature. The aqueous layer was extracted with CH2Cl2 (3×10 mL). The resulting mixture was concentrated under reduced pressure. The residue was purified by Pep-TLC eluted with CH2Cl2/MeOH (10:1) to afford 269d (90 mg, 43.27%) as a light yellow solid.

### 5. Synthesis of 269e

Into a 8 ml, sealed tube were added 269d (90 mg, 0.133 mmol, 1 equiv), DCM (1.5 mL) and TFA (0.3 mL) at room temperature. The resulting mixture was stirred for 2 h at room temperature. The resulting mixture was concentrated under reduced pressure. The residue was purified by Prep-TLC eluted with CH2Cl2/MeOH (10:1) to afford 269e (40 mg, 57.17%) as a light yellow solid.

### 6. Synthesis of 269

269e (40 mag) was purified by chiral separation with the following conditions (Column: CHIRALPAK IC, 2*25 cm, 5 μm; Mobile Phase A: MtBE (0.5% 2M NH3-MeOH)-HPLC, Mobile Phase B: MeOH:DCM=1:1-HPLC; Flow rate: 20 mL/min; Gradient: 10% B to 10% B in 8 min; Wave Length: 220/254 nm; RT1 (min): 5.66; RT2 (min): 7.25; The first peak was the product. Sample Solvent: MeOH:DCM=1:1; Injection Volume: 0.55 mL; Number of Runs: 4) to afford 269 (11.7 mg, 29.25%) as a light yellow solid.

H-NMR: 1H NMR (300 MHz, CD30D-d4) S 0.90-0.94 (m, 4H), 1.60-2.10 (m, 12H), 2.86-2.92 (m, 2H), 3.25-3.28 (m, 1H), 3.32-3.34 (m, 2H), 4.22-4.25 (d, 1H), 7.13-7.16 (d, 2H), 7.34-7.36 (d, 1H), 7.45-7.49 (m, 1H), 7.61-7.62 (d, 1H), 7.64-7.68 (d, 2H), 8.21 (s, 1H).

### Example 270. Synthesis of Compound 270

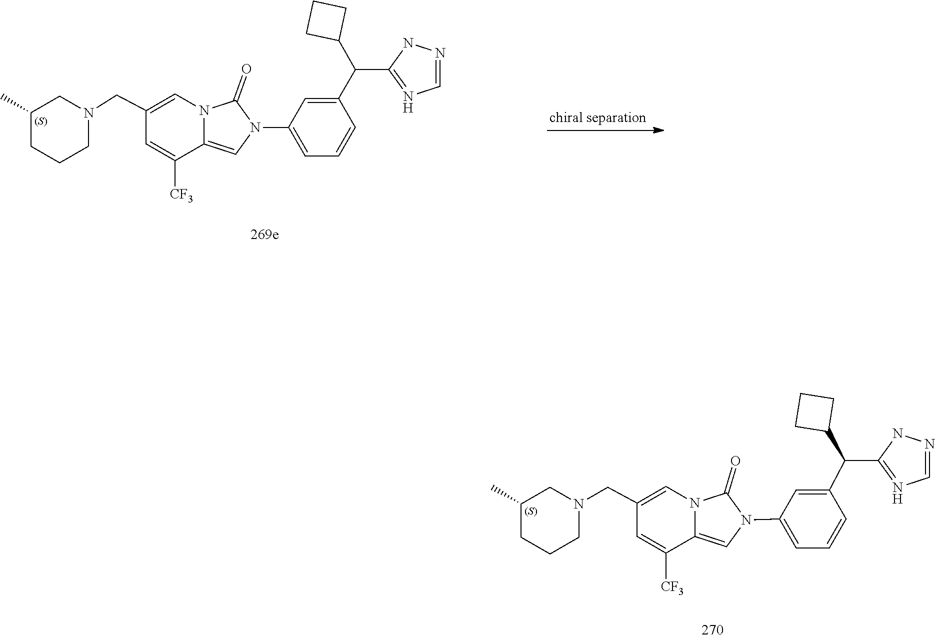

### Synthesis of 270

269e (40 mg) was purified by chiral separation with the following conditions (Column: CHIRALPAK IC, 2*25 cm, 5 μm; Mobile Phase A: MtBE (0.5% 2M NH3-MeOH)-HPLC, Mobile Phase B: MeOH:DCM=1:1-HPLC; Flow rate: 20 mL/min; Gradient: 10% B to 10% B in 8 min; Wave Length: 220/254 un; RT1 (min): 5.66; RT2 (min): 7.25; The second peak was the product. Sample Solvent: MeOH:DCM=1:1; Injection Volume: 0.55 mL; Number of Runs: 4) to afford 270 (11.9 mg, 29.75%) as a light yellow solid.

H-NMR-270 1H NMR (300 MHz, CD30D-d4) δ 0.91-0.95 (m, 4H), 1.61-2.11 (m, 12H), 2.86-2.92 (m, 2H), 3.25-3.28 (m, 1H), 3.32-3.34 (m, 2H), 4.22-4.25 (d, 1H), 7.13-7.16 (d, 2H), 7.34-7.36 (d, 1H), 7.45-7.49 (m, 1H), 7.61-7.62 (d, 1H), 7.64-7.68 (d, 2H), 8.23 (s, 1H).

### Example 271. Synthesis of Compound 271

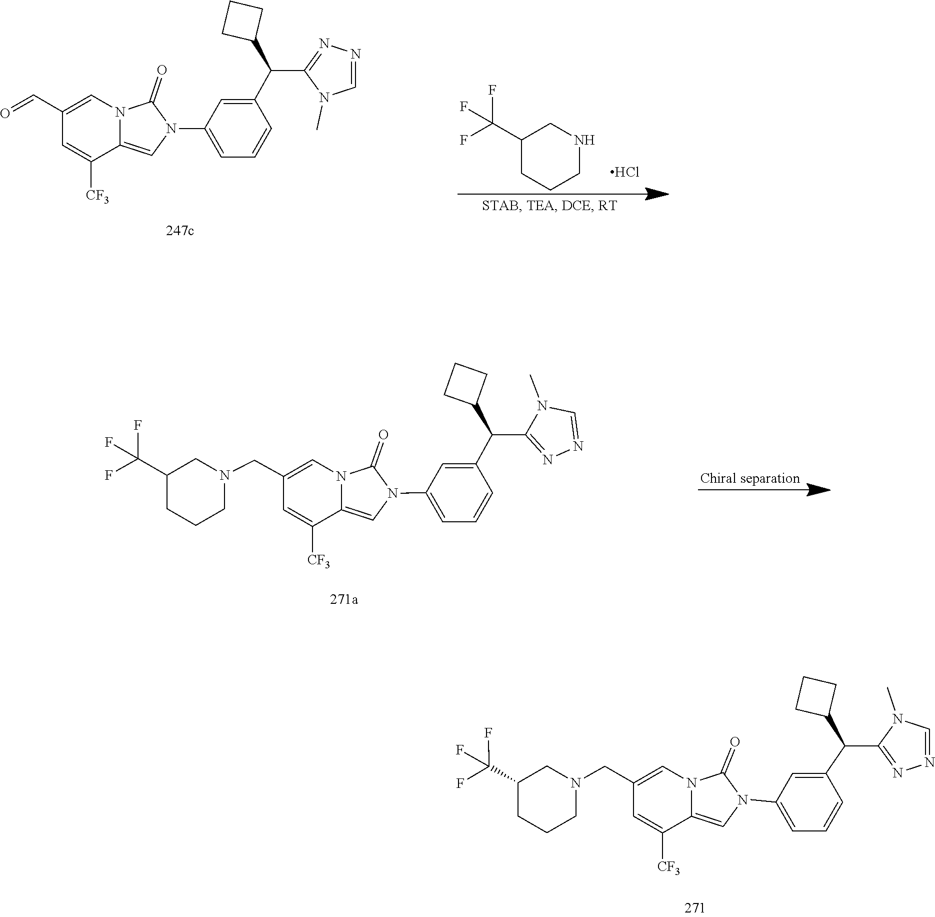 [SMILES: Cn1cnnc1[C@@H](c1cccc(-n2cc3c(C(F)(F)F)cc(C=O)cn3c2=O)c1)C1CCC1 ; FC(F)(F)C1CCCNC1 ; Cl ; Cn1cnnc1[C@@H](c1cccc(-n2cc3c(C(F)(F)F)cc(CN4CCCC(C(F)(F)F)C4)cn3c2=O)c1)C1CCC1 ; Cn1cnnc1[C@@H](c1cccc(-n2cc3c(C(F)(F)F)cc(CN4CCC[C@H](C(F)(F)F)C4)cn3c2=O)c1)C1CCC1]

### 1. Synthesis of 271a

To a stirred solution of 247c (500 mg, 1,098 mmol, 1.00 equiv) and 3-(trifluoromethyl)piperidine hydrochloride (416.32 mg, 2.196 mmol, 2 equiv) in DCE (6 mL) were added TEA (222.19 mg, 2.196 mmol, 2 equiv) at room temperature. The mixture was stirred for 30 min at room temperature, To the above mixture was added STAB (465.35 mg, 2.196 mmol, 2 equiv) at room temperature. The resulting mixture was stirred for overnight at room temperature. The reaction was quenched by the addition of sat. NH4Cl (aq.) (15 mL) at room temperature. The resulting mixture was extracted with CH2Cl2 (3×25 mL). The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/ruin; Gradient: 45% B to 70% B in 7 min, Wave Length: 220 nm; RT1 (min): 6.44) to afford 271a (210 mg, 30.99%) as a yellow solid.

### 2. Synthesis of 271

271a (300 mg) was purified by Chiral separation with the following conditions (Column: CHIRALPAK IG, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 25% B to 25% B in 17 min; Wave Length: 220/254 nm; RT1 (min): 11.23; RT2 (min): 14.24; The first peak was the product. Sample Solvent: EtOH:DCM=1:1-HPLC; Injection Volume: 0.3 mL; Number of Runs: 11). This resulted in 271 (61.2 mg, 28.62%) as a yellow solid.

LC-MS: (ES, m/z): [M+H]+: 593. H-NMR: (400 MHz, DMSO-d6, ppm): δ 1.21-1.27 (m, 2H) δ 1.45-1.52 (m, 1H), δ 1.63-1.89 (m, 8H), δ 1.92-2.14 (m, 3H), δ 2.80 (s, 1H), δ 2.97 (s, 1H), δ 3.25 (s, 1H), δ 3.46 (s, 4H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 3H), δ 8.35 (s, 1H).

### Example 272. Synthesis of Compound 272

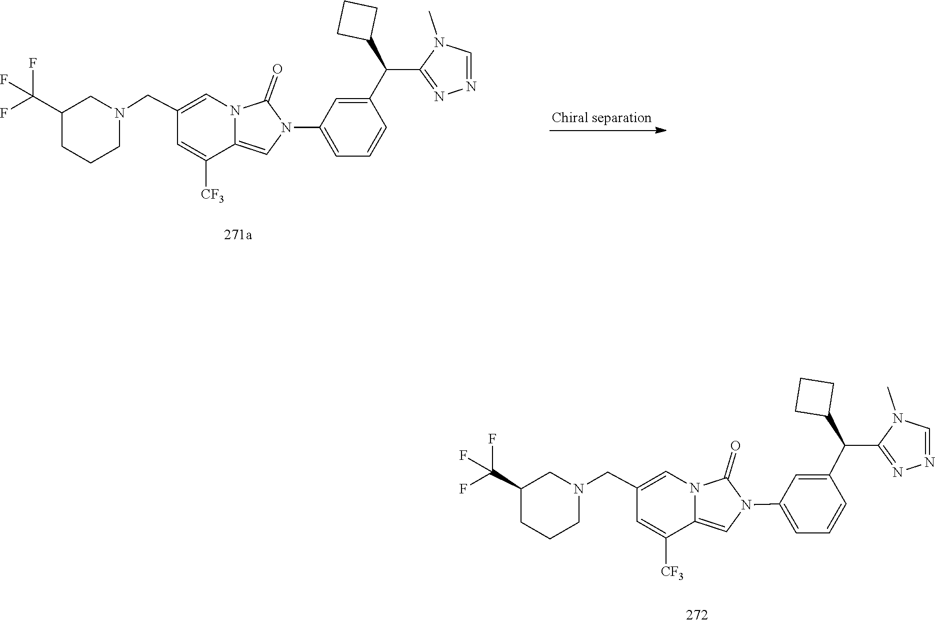 [SMILES: Cn1cnnc1[C@@H](c1cccc(-n2cc3c(C(F)(F)F)cc(CN4CCCC(C(F)(F)F)C4)cn3c2=O)c1)C1CCC1 ; Cn1cnnc1[C@@H](c1cccc(-n2cc3c(C(F)(F)F)cc(CN4CCC[C@@H](C(F)(F)F)C4)cn3c2=O)c1)C1CCC1]

### Synthesis of 270

271a (300 mg) was purified by Chiral separation with the following conditions (Column: CHIRALPAK IG, 2*25 cm, 5 um; Mobile Phase A: Hex (0.5% 2M NH3-MeOH)-HPLC, Mobile Phase B: EtOH:DCM=1:1-HPLC; Flow rate: 20 mL/min; Gradient: 25% B to 25% B in 17 min; Wave Length: 220/254 nm; RT1 (min): 11.23; R T2 (min): 14.24; The second peak was the product. Sample Solvent: EtOH:DCM=1:1-HPLC; Injection Volume: 0.3 mL; Number of Runs: 11). This resulted in 272 (65.5 mg, 30.88%) as a yellow solid.

LC-MS: (ES, m/z): [M+H]+: 593. H-NMR: (400 MHz, DMSO-d6, ppm): δ 1.21-1.27 (m, 2H) S 1.45-1.52 (m, 1H), δ 1.63-1.89 (m, 8H), δ 1.92-2.14 (m, 3H), δ 2.80 (s, 1H), δ 2.97 (s, 1H), δ 3.25 (s, 1H), δ 3.46 (s, 4H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 3H), δ 8.35 (s, 1H).

### Example 273. Synthesis of Compound 273

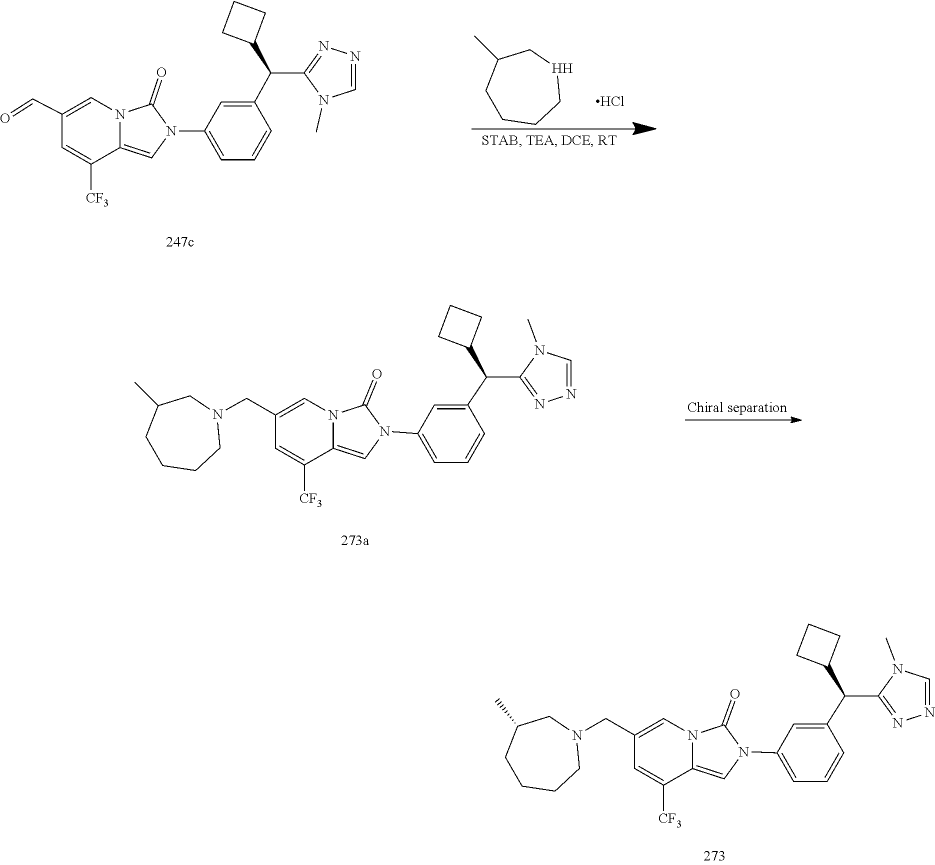

### 1. Synthesis of 273a

To a stirred mixture of 3-methylazepane hydrochloride (394.33 mg, 2,634 mmol, 2 equiv) and 247c (600 mg, 1.317 mmol, 1.00 equiv) in DCE (6 mL) was added Et3N (399.94 mg, 3.951 mmol, 3 equiv) at room temperature. The resulting mixture was stirred for 30 min at room temperature. To the above mixture was added NaBH(OAc)3 (837.63 mg 3.951 mmol, 3 equiv) at room temperature. The resulting mixture was stirred for additional 4 h at room temperature. The reaction was quenched by the addition of water (30 mL) at room temperature. The aqueous layer was extracted with EtOAc (2×15 mL). The resulting mixture was concentrated under reduced pressure. The residue was purified by Prep-TLC with CH2Cl2/MeOH=15:1 to afford the crude product (400 mg) as a yellow solid. The crude product (400 mg) was purified by Prep-HPLC with the following conditions (Column: YMC-Actus Triart C18 ExRS, 30*150 mm, 5 μm; Mobile Phase A: water (10 mmol/L NH4HCO3), Mobile Phase B: ACN; Flow rate: 60 mL/min; Gradient: 62% B to 87% B in 8 min, 87% B; Wave Length: 220 nm; RT1 (min): 7.77) to afford 273a (170 mg, 22.42%) as a yellow solid.

### 2. Synthesis of 273

273a (170 mg) was separated by Prep-SFC with the following conditions (Column: CHIRALPAK IG, 3*25 cm, 5 um; Mobile Phase A: C02, Mobile Phase B: MeOH (0.1% 2M NH3-MeOH); Flow rate: 100 mL/min; Gradient: isocratic 35% B; Column Temperature (° C.): 35; Back Pressure (bar): 100; Wave Length: 220 nm; RT1 (min): 9.78; RT2 (min): 11.6; the first peak was product) to afford 273 (51.6 mg, 29.96%) as a yellow solid.

LCMS: (ES, m/z): [M+H]⁺ 553. H-NMR: (400 MHz, DMSO-d6, ppm, δ): 0.80 (s, 3H), 1.12-1.25 (m, 1H), 1.40-1.52 (m, 2H), 1.52-1.81 (m, 9H), 2.05-2.15 (m, 1H), 2.22-2.28 (m, 1H), 2.55-2.67 (m, 3H), 3.15-3.25 (m, 1H), 3.42-3.43 (m, 5H), 4.24-4.27 (d, 1H), 7.04 (s, 1H), 7.18-7.20 (d, 1H), 7.30 (s, 1H), 7.42-7.46 (m, 1H), 7.67-7.70 (m, 2H), 7.74 (s, 1H), 8.32 (s, 1H).

### Example 274. Synthesis of Compound 274

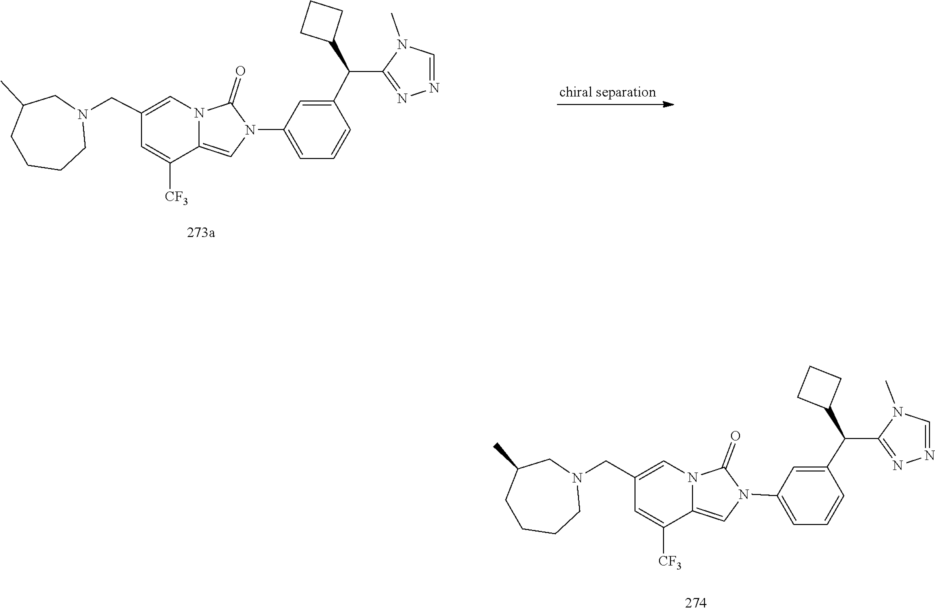 [SMILES: CC1CCCCN(Cc2cc(C(F)(F)F)c3cn(-c4cccc([C@H](c5nncn5C)C5CCC5)c4)c(=O)n3c2)C1 ; C[C@@H]1CCCCN(Cc2cc(C(F)(F)F)c3cn(-c4cccc([C@H](c5nncn5C)C5CCC5)c4)c(=O)n3c2)C1]

### Synthesis of 274

273a (170 mag) was separated by Prep-SFC with the following conditions (Column: CHIRALPAK IG, 3*25 cm, 5 um; Mobile Phase A: C02, Mobile Phase B: MeOH (0.1% 2M NH3-MeOH); Flow rate: 100 mL/min; Gradient: isocratic 35% B; Column Temperature (° C.): 35; Back Pressure (bar): 100; Wave Length: 220 nm; RT1 (min): 9.78; RT2 (min): 11.6; the second peak is product) to afford 274 (61.0 mg, 34.34%) as a yellow solid.

LCMS: (ES, m/z): [M+H]⁺ 553. H-NMR: (400 MHz, DMSO-d6, ppm, δ): 0.80 (s, 3H), 1.12-1.25 (m, 1H), 1.40-1.52 (m, 2H), 1.52-1.81 (m, 9H), 2.05-2.15 (m, 1H), 2.22-2.28 (m, 1H), 2.55-2.67 (m, 3H), 3.15-3.25 (m, 1H), 3.42-3.43 (m, 5H), 4.24-4.27 (d, 1H), 7.04 (s, 1H), 7.18-7.20 (d, 1H), 7.30 (s, 1H), 7.42-7.46 (t, 1H), 7.67-7.74 (m, 3H), 8.32 (s, 1H).

### Example 275. Synthesis of Compound 275

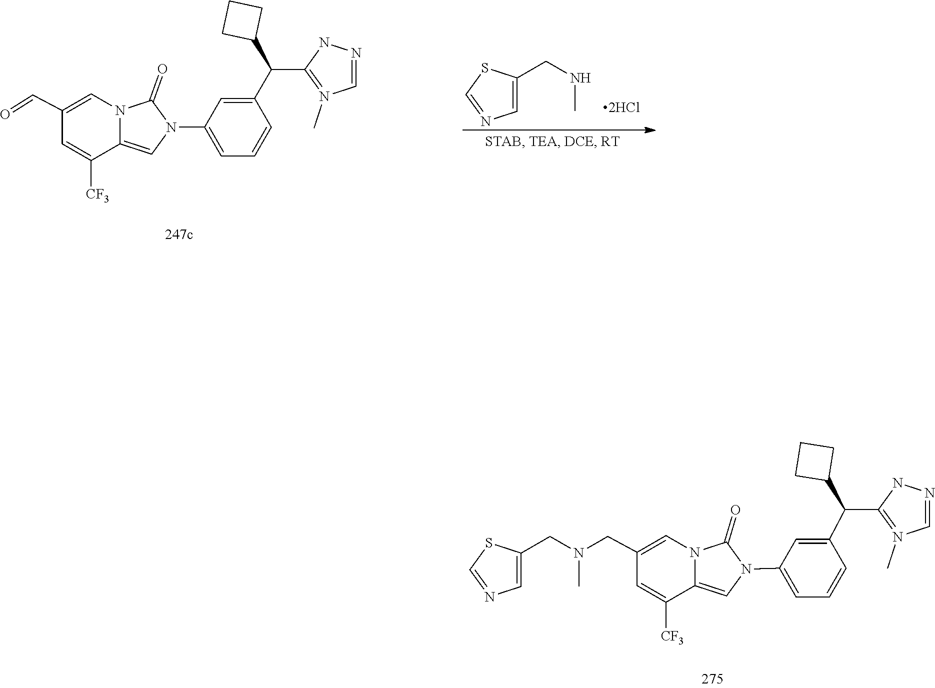

### Synthesis of 275

To a stirred solution of 247c (200 mg, 0,439 mm ol, 1 equiv) and Methyl-thiazol-5-ylmethyl-amine dihydrochloride (175.65 mag, 0.878 mmol, 2 equiv) in DCE (5 mL) was added TEA (177.75 mg, 1.756 mmol, 4 equiv) at room temperature under nitrogen atmosphere. The resulting mixture was stirred for 1h at room temperature. To the above mixture was added STAB (186.14 mg, 0.878 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The organic layers were concentrated under reduced pressure. The residue was purified by Prep-TLC (DCM/MeOH 20:1) to afford 275 (45.1 mg, 17.79%) as a yellow solid.

LC-MS: (ES, m/z): [M+H]+568. H-NMR: (400 MHz, DMSO-d6, δ ppm): 1.63-1.83 (m, 5H), 2.03-2.15 (m, 1H), 2.21 (s, 3H), 3.19-3.29 (m, 1H), 3.34 (s, 2H), 3.42 (s, 3H), 3.84 (s, 2H), 4.25-4.27 (d, 1H), 7.01 (s, 1H), 7.18-7.20 (d, 1H), 7.32 (s, 1H), 7.42-7.46 (m, 1H), 7.68-7.79 (m, 3H), 7.80 (s, 1H), 8.32 (s, 1H), 9.04 (s, 1H).

### Example 276. Synthesis of Compound 276

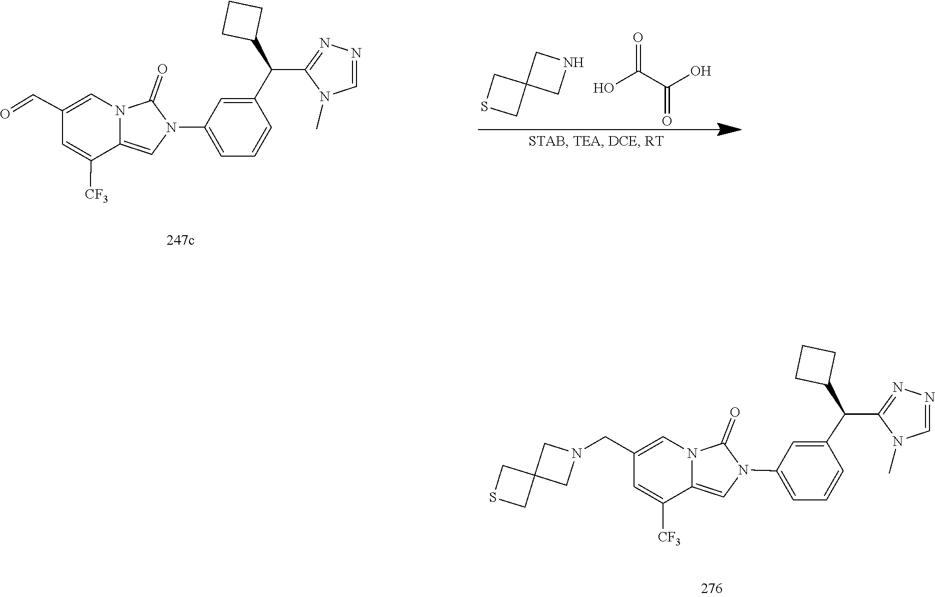 [SMILES: Cn1cnnc1[C@@H](c1cccc(-n2cc3c(C(F)(F)F)cc(C=O)cn3c2=O)c1)C1CCC1 ; C1NCC12CSC2 ; O=C(O)C(=O)O ; Cn1cnnc1[C@@H](c1cccc(-n2cc3c(C(F)(F)F)cc(CN4CC5(CSC5)C4)cn3c2=O)c1)C1CCC1]

### Synthesis of 276

To a stirred solution of 247c (300 mg, 0.659 mmol, 1.00 equiv) and 2-thia-6-azaspiro[3.3]heptane hemioxalate (405.10 mg, 1.977 mmol, 3 equiv) in DCE (5 mL) was added TEA (199.97 mg, 1.977 mmol, 3 equiv) at room temperature. The resulting mixture was stirred for 1h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 ml). The organic layers were concentrated under reduced pressure. The residue was purified by Prep-TLC (DCM/MeOH 20:1) to afford the crude product. The residue was purified by reverse flash chromatography with the following conditions: column, C18 silica gel; mobile phase A: water (1O mmol/L NH4HCO3), B: MeCN, 50% B to 60% B gradient in 20 min; detector, UV 254 nm. This resulted in 276 (122.7 mg, 33.11%) as a yellow solid.

LC-MS-276 (ES, m/z): [M+H]+555. H-NMR-276 (400 MHz, DMSO-d6, δ ppm): 1.63-1.93 (m, 5H), 2.03-2.15 (m, 1H), 3.21-3.25 (m, 5H), 3.28-3.32 (m, 6H), 3.43 (s, 3H), 4.25-4.27 (d, 1H), 6.94 (s, 1H), 7.18-7.20 (d, 1H), 7.31 (s, 1H), 7.42-7.46 (m, 1H), 7.62-7.74 (m, 3H), 8.33 (s, 1H).

### Example 277. Synthesis of Compound 277

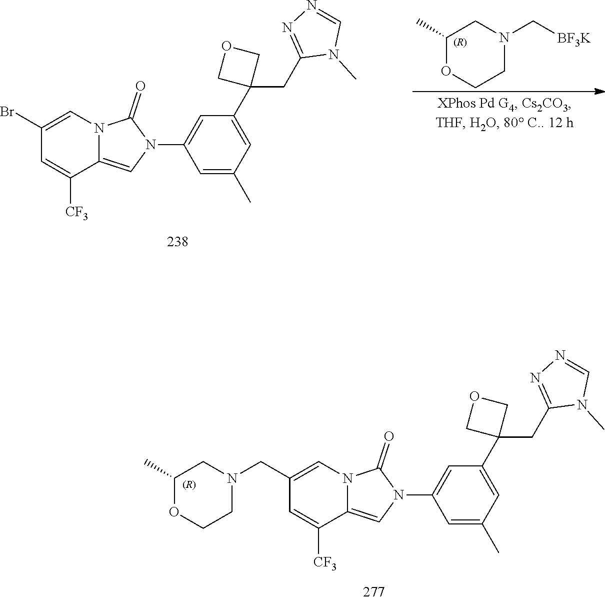

### Synthesis of 277

To a solution of 238 (210 mg, 402.05 umol, 1 equiv) and potassium hydride;trifluoro-[[(2R)-2-methylmorpholin-4-yl]methyl]boron (177 mg, 804 umol, 2 equiv) in water (4 mL) and THF (16 mL) was added dicyclohexyl-[2-(2,4,6-triisopropylphenyl)phenyl]phosphane;methanesulfonate;[2-[2-(methylamino)phenyl]phenyl]palladium(1+) (34.6 mg, 40.2 umol, 0.1 equiv) and Cs2CO3 (393 mg, 121 mmol, 3 equiv). The mixture was stirred at 80° C. for 12 h under nitrogen atmosphere. The mixture was diluted with water (100 ml) and extracted with EtOAc (50 ml×3). The combined organic was dried over anhydrous Na2SO4, filtered and the filtrate was concentrated under reduced pressure to give a residue. The residue was purified by Prep-HPLC with the following conditions (Column: Phenomenex Luna C18 150*30 mm*5 um; Mobile Phase A: Water (0.2% formic acid), Mobile Phase B: ACN; Flow rate: 35 mL/min; Gradient: 1% B to 35% B in 8 rain; Wave Length: 220 nm; R T (min): 8.0) to afford 277 (4.8 mg, 2%) as a yellow solid.

LCMS: (ES, m/z): [M+H]+557.3. H-NMR: (400 MHz, DMSO-d6) δ 8.20 (s, 1H), 7.67 (s, 1H), 7.56 (s, 1H), 7.28 (s, 1H), 7.21 (s, 1H), 7.01 (s, 1H), 6.71 (s, 1H), 4.94-4.88 (m, 4H), 3.78-3.70 (m, 1H), 3.51 (s, 2H), 3.49 (d, J=2.0 Hz, 1H), 3.25 (d, J=4.4 Hz, 2H), 2.98 (s, 3H), 2.74 (d, J=10.8 Hz, 1H), 2.65 (d, J=0.8 Hz, 1H), 2.43-2.37 (m, 1H), 2.29 (s, 3H), 2.09-2.02 (m, 1H), 1.74 (t, J=10.4 Hz, 1H), 1.04 (d, J=6.4 Hz, 3H)

### Example 278. Synthesis of Compound 278_P1 & 278_P2

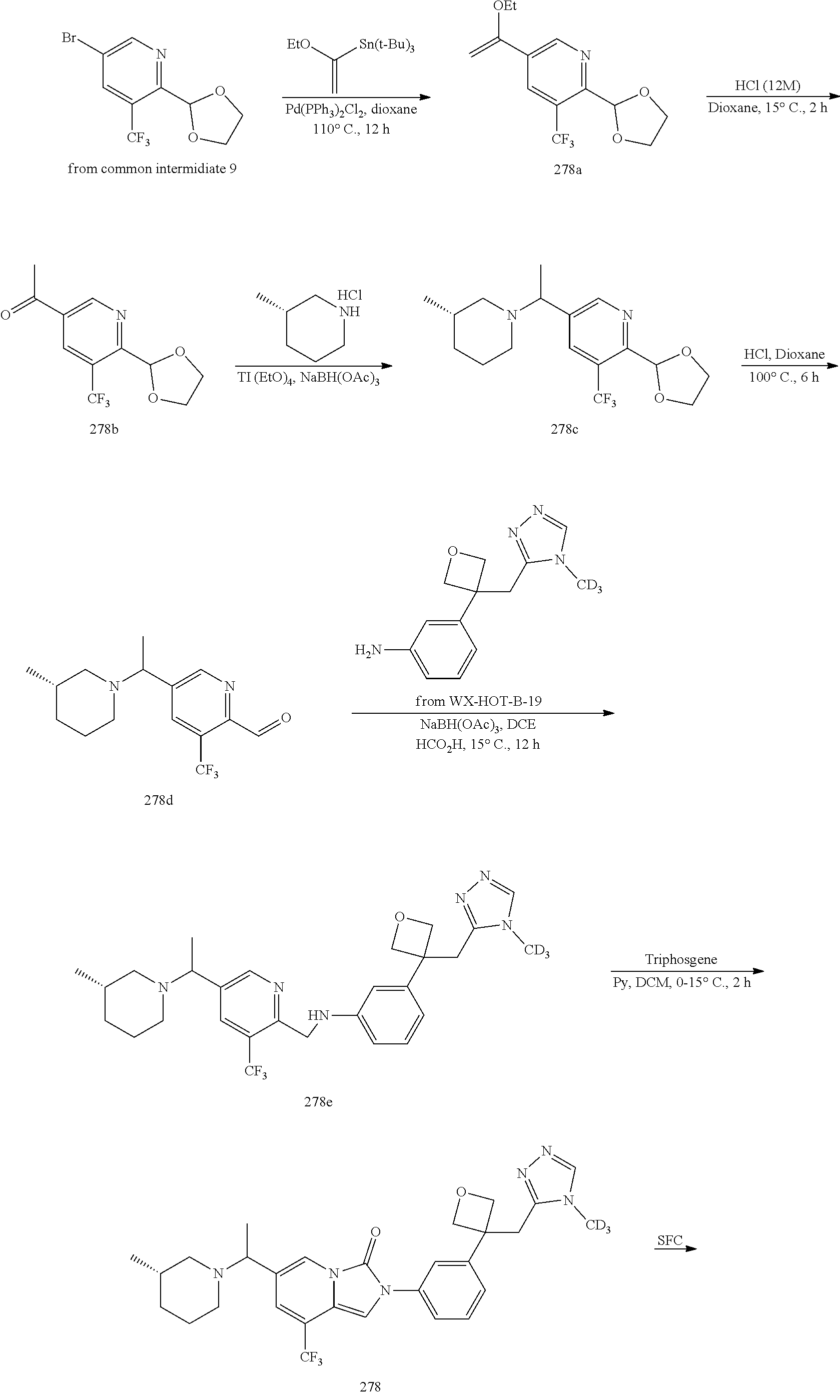

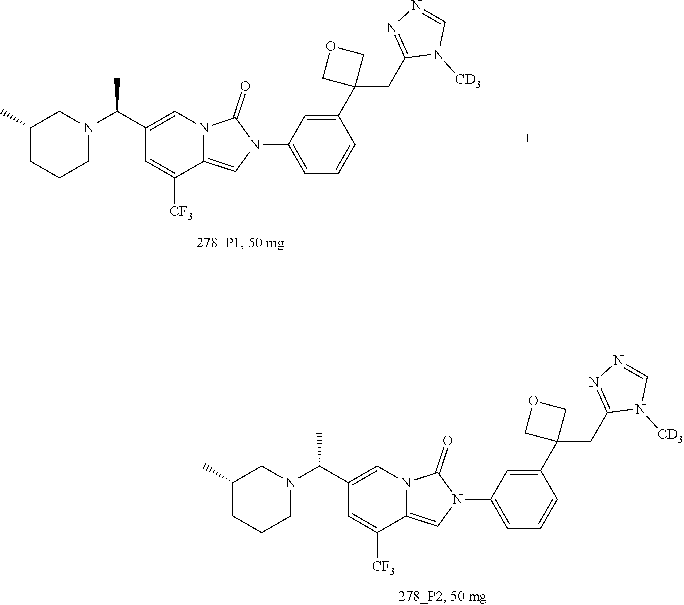 [SMILES: [2H]C([2H])([2H])n1cnnc1CC1(c2cccc(-n3cc4c(C(F)(F)F)cc([C@H](C)N5CCC[C@H](C)C5)cn4c3=O)c2)COC1 ; [2H]C([2H])([2H])n1cnnc1CC1(c2cccc(-n3cc4c(C(F)(F)F)cc([C@@H](C)N5CCC[C@H](C)C5)cn4c3=O)c2)COC1]

### 1. Synthesis of 278a

A mixture of 5-bromo-2-(1,3-dioxolan-2-yl)-3-(trifluoromethyl)pyridine (10 g, 33.55 mmol, equiv), tritert-butyl(1-ethoxyvinyl)stannane (13.33 g, 36.91 mmol, 1.1 equiv), Pd(Ph3)2Cl2 (1.18 g, 1.68 mmol, 0.05 equiv) in dioxane (100 mL) was degassed and purged with N2 for 3 times, and then the mixture was stirred at 110° C. for 12 hr under N2 atmosphere. The reaction mixture was quenched by addition saturated KF 100 mL and stirred at r.t. for 1 hr under N2. Then it was diluted with H2O 500 mL and extracted with DCM (2*100 mL). The combined organic layers were dried over anhydrous sodium sulfate, filtered and the filtrate was concentrated under reduced pressure to give a residue. The residue was purified by column chromatography (SiO2, Petroleum ether/Ethyl acetate=50/1 to 0/1) to give 278a (7 g, 24.20 mmol, 72.13% yield) as a yellow oil.

### 2. Synthesis of 278b

A mixture of 278a (7 g, 24.20 mmol) in HCl (1 ml, 12 M) and dioxane (50 mL) was degassed and purged with N2 for 3 times, and then the mixture was stirred at r.t. for 2 hr under N2 atmosphere. The reaction mixture was quenched by addition Na2CO3 aq. 100 mL and extracted with DCM (2*250 mL). The combined organic layers were dried over anhydrous sodium sulfate, filtered and the filtrate was concentrated under reduced pressure to give a residue. The residue was purified by column chromatography (SiO2, Petroleum ether/Ethyl acetate=50/1 to 0/1) to give 278b (5 g, 19.14 mmol, 79.10% yield) as a yellow solid.

H-NMR-278b: (400 MHz, CDCl3): δ 9.25 (d, J=2 Hz, 1H), 8.44 (d, J=1.2 Hz, 1H), 6.25 (s, 1H), 4.27 (t, J=3.6 Hz, 214), 3.08 (t, J=3.2 Hz, 2H), 2.62 (s, 3H).

### 3. Synthesis of 278c

A mixture of 278b (2.5 g, 9.57 mmol, 1 equiv), (3S)-3-methylpiperidine (1.42 g, 14.36 mmol, 1.5 equiv), Ti(i-PrO)4 (10.88 g, 38.29 mmol, 4 equiv) in DCE (30 mL) was stirred at 80° C. for 6 hr under N2 atmosphere. Then it was cooled to room temperature and the reaction mixture was added NaBH(OAc)3 (6.09 g, 28.71 mmol, 3 equiv). The reaction mixture was heated to 80° C. and stirred at 80° C. for 6 hr. The reaction mixture was quenched by addition H2O 100 ml, filtered and the filtrate was extracted with DCM (2*200 mL). The combined organic layers were dried over anhydrous sodium sulfate, filtered and the filtrate was concentrated under reduced pressure to give a residue. The residue was purified by column chromatography (SiO2, Petroleum ether/Ethyl acetate=50/1 to 0/1) to give 278c (1 g, 2.90 mmol, 30.34% yield) as a yellow solid.

H-NMR: (400 MHz, MeOD): δ 8.78 (s, 1H), 8.12 (s, 1H), 6.17 (s, 1H), 4.29-424 (m, 2H), 4.11-4.05 (m, 2H), 3.71 (d, J=6.8 Hz, 1H), 2.94-2.92 (m, 1H), 2.73-2.60 (m, 1H), 1.93-1.69 (m, 1H), 1.68-1.60 (m, 6H), 1.43 (dd, J=6.8, 1.4 Hz, 3H), 0.88 (d, 1=6.00 Hz, 2H), 0.81 (d, J=6.00 Hz, 1H)

### 4. Synthesis of 278d

A mixture of 278c (1 g, 2.90 mmol, 1 equiv) in dioxane (4 mL) and HCl (4 ml, 4M) was degassed and purged with N2 for 3 times, and then the mixture was stirred at 100° C. for 6 hr under N2 atmosphere. The reaction mixture was quenched by addition Na2CO3 aq. 20 ml and extracted with DCM (2* 200 mL). The combined organic layers were dried over anhydrous sodium sulfate, filtered and the filtrate was concentrated under reduced pressure to give a residue. The crude product 278d (550 mg, 1.83 mmol, 63.07% yield) was obtained as yellow oil and used into the next step without further purification.

### 5. Synthesis of 278e

To a mixture of 278d (550 mg, 1.83 mmol, 1 equiv), 3-[3-[[4-(trideuteriomethyl)-1,2,4-triazol-3-yl]methyl]oxetan-3-yl]aniline (475.57 mg, 1.92 mmol, 1° 05 equiv) in HCOOH (87.98 mg, 1.83 mmol, 1 equiv) and DCE (10 mL) was added NaBH(OAc)3 (776.29 mg, 3.66 mmol, 2 equiv). The mixture was stirred at r.t. for 12 hr under N2 atmosphere. The reaction mixture was quenched by addition H2O 20 mL and extracted with DCM (2* 200 mL). The combined organic layers were dried over anhydrous sodium sulfate, filtered and the filtrate was concentrated under reduced pressure to give a residue. The residue was purified by column chromatography (SiO2, DCM: methyl alcohol=50/1-1:1) to give 278e (300 mg, 564.30 umol, 30.81% yield) was obtained as a yellow solid.

H-NMR 5: (400 MHz, MeOD): δ 8.63 (s, 1H), 8.08-8.01 (m, 2H) 6.96-6.91 (m, 1H), 6.50-6.48 (m, 1 H), 5.99-5.98 (m, 1H), 5.97-5.92 (m, 1H), 4.87 (t, J=2.8 Hz, 2H), 4.83 (t, J=4.8 Hz, 2H), 4.37 (s, 2H), 373-3.71 (m, 1H), 3.44 (d, J=4.4 Hz, 2H), 2.99-2.91 (i, 1 H), 2.70-2.67 (m, 1H), 1.93-1.91 (m, 1H), 1.63-1.59 (m, 6H), 1.39 (d, J=6.8 Hz, 3H), 0.79 (d, 3=6.0 Hz, 2H), (d, J=6.4 Hz, 1H).

### 6. Synthesis of 278

A mixture of 278e (300 mg, 564.30 umol, 1 equiv), Py. (223.18 mg, 2.82 mmol, 5 equiv) in DCM (10 mL) was added TRIPHOSGENE (184.20 mg, 620.73 umol, 1.1 equiv) under N2. The mixture was stirred at 0° C. for 2 hr under N2 atmosphere. The reaction mixture was quenched by addition a.q. NaHCO3 50 mL at 0° C. under N2 and extracted with DCM (2*200 mL). The combined organic layers were dried over anhydrous sodium sulfate, filtered and the filtrate was concentrated under reduced pressure to give a residue. The residue was purified by prep-HPLC (column: Waters Xbridge Prep OBD C18 150*40 mm*10 um; mobile phase: [water (NH4HCO3)-ACN]; B %: 35%-65%, 8 min) to give 278 (85 mg, 143.47 umol, 25.42% yield) was obtained as a yellow solid.

H-NMR-278: (400 MHz, d6-DMSO): δ 8.25 (s, 1H), 7.82-7.79 (m, 1H), 7.62 (m, 1H), 7.46 (t, J=8 Hz, 2H), 7.36 (s, 1H), 7.13 (s, 1H), 6.95 (d, J=8 Hz, 1H), 5.02-4.94 (m, 4H), 3.59 (s, 2H), 3.51-3.46 (m, 1H), 2.81-2.77 (m, 2H), 2.07-1.98 (m, 1H), 1.72-1.67 (m, 5H), 1.64-1.63 (m, 1H), 1.31-1.29 (m, 3H), 0.89-0.87 (m, 3H).

### Synthesis of 278_P1&P2

85 mg of 278 was separated by SFC (column: REGIS(S,S)WHELK-01 (250 mm*25 mm, 10 um); mobile phase: [0.1% NH320 IPA]; B %: 55%-55%, 8 min) to give 278_P1 (23.5 mg) as a yellow solid and 278_P2 (22 mg) as a yellow solid.

MS-278_P1: (ES, m/z): [M+H]+558.3.

H-NMR-278_P1: (400 MHz, DMSO-d6): δ 8.20 (s, 1H), 7.76-7.74 (m, 1H), 7.56 (s, 1H), 7.41 (t, J=7.6 Hz, 2H), 7.30 (s, 1H), 7.08 (s, 1H), 6.90 (d, J=7.6 Hz, 1H), 4.96-4.91 (m, 4H), 3.53 (s, 2H), 3.45-3.41 (m, 1H), 2.75-2.73 (m, 2H), 1.97-1.90 (m, 1H), 1.67-1.56 (m, 5H), 1.45-1.36 (m, 1H), 1.25-1.24 (m, 3H), 0.83-0.81 (m, 3H).

MS-278_P2: (ES, m/z): [M+H]+558.3.

H-NMR-278_P2: (400 MHz, DMSO-d6): δ 8.20 (s, 1H), 7.76-7.74 (m, 1H), 7.56 (s, 1H), 7.41 (t, J=7.6 Hz, 2H), 7.30 (s, 1H), 7.08 (s, 1H), 6.90 (d, J=7.6 Hz, 1H), 4.96-4.91 (m, 4H), 3.53 (s, 2H), 3.45-3.41 (m, 1H), 2.75-2.73 (m, 2H), 1.97-1.90 (m, 1H), 1.67-1.56 (m, 5H), 1.45-1.36 (m, 1H), 1.25-1.24 (m, 3H), 0.85-0.83 (m, 3H).

### Example 279. Synthesis of Compound 279

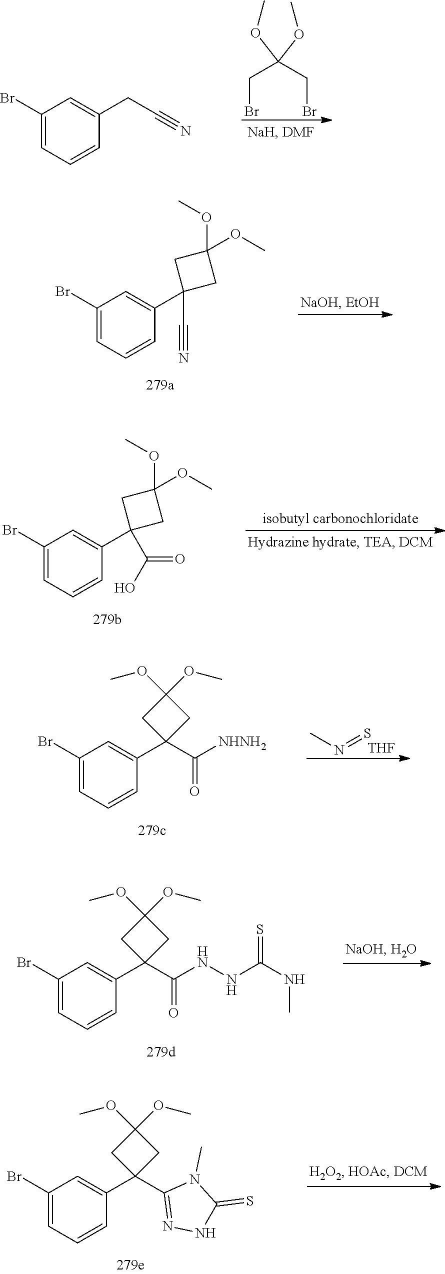

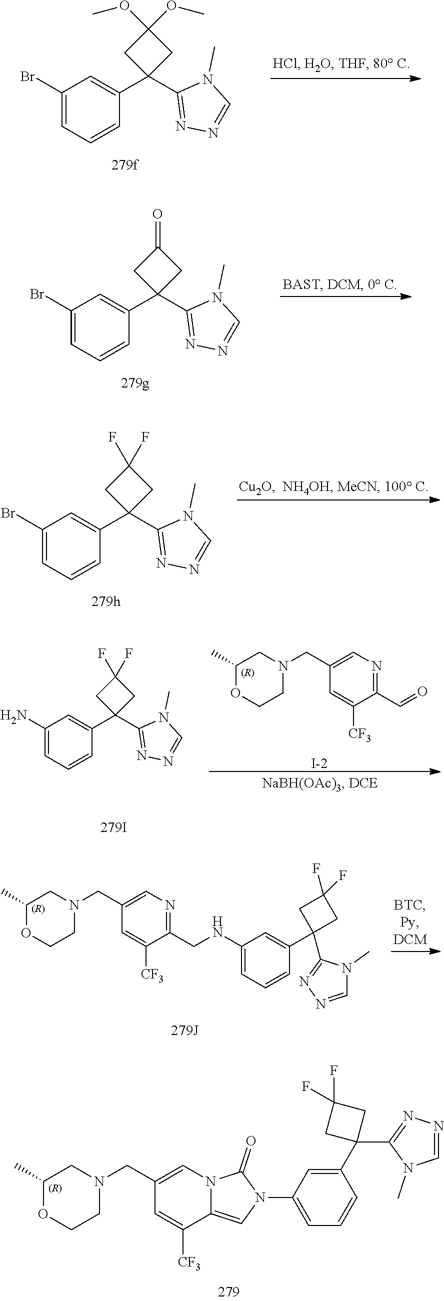

### 1. Synthesis of 279a

To a stirred solution of NaH (36.54 g, 913,544 mmol, 2 equiv, 60%) in DMF (1000 mL) was added 2-(2-bromopyridin-4-yl)acetonitrile (90 g, 456,772 mmol, 1 equiv) dropwise at 0° C. under nitrogen atmosphere, followed by 1,3-dibromo-2,2-dimethoxypropane (100 g, 381.765 mmol, 0.84 equiv) at 60° C. under nitrogen atmosphere. The reaction was quenched with saturated NH4Cl (aq.) (3 L) at room temperature. The aqueous layer was extracted with EtOAc (2×500 mL). The organic layers were concentrated under reduced pressure. The residue was purified by silica gel column chromatography, eluted with PE/EA (10:1) to afford 279a (50 g, 34.37%) as an off-white solid.

### 2. Synthesis of 279b

To a stirred solution of 279a (50 g, 168.825 mmol, 1 equiv) in EtOH (600 mL) was added NaOH (23.63 g, 590,887 mmol, 3.5 equiv) in 1120 (600 mL) at room temperature. The resulting mixture was stirred overnight at 80° C. The EtOH was concentrated under vacuum. The mixture was acidified to pH 2 with HCl (2M). The aqueous layer was extracted with EtOAc (4×200 mL). The combined organic layers were dried over anhydrous Na2SO4. After filtration, the filtrate was concentrated under reduced pressure. This resulted in 279b (44 g, 75.25%) as an off-white solid,

### 3. Synthesis of 279c

To a stirred solution of 279b (44 g, 139.610 mmol, 1 equiv) and TEA (28.26 g, 279.220 mmol, 2 equiv) in DCM (1500 mL) was added isobutyl carbonochloridate (28.60 g, 209.415 mmol, 1.5 equiv) dropwise at 0° C. under nitrogen atmosphere. The resulting mixture was stirred for 30 min at 0° C., To the above mixture was added Hydrazine hydrate (27.96 g, 558.440 mmol, 4 equiv) dropwise at −30° C. The resulting mixture was stirred for additional 40 min at room temperature. The reaction was quenched with water (2 L) at room temperature. The aqueous layer was extracted with DCM (3×300 mL). The combined organic layers were dried over anhydrous Na2SO4. After filtration, the filtrate was concentrated under reduced pressure. This resulted in 279c (45 g, 88.12%) as an off-white solid,

### 4. Synthesis of 279d

To a stirred solution of 279c (45 g, 136.698 mmol, 1 equiv) in tetrahydrofuran (500 mL) was added methyl isothiocyanate (29.98 g, 410.094 mmol, 3 equiv) at room temperature. The resulting mixture was stirred overnight at room temperature. The resulting mixture was diluted with water (400 mL). The resulting mixture was concentrated under vacuum. The precipitated solids were collected by filtration and washed with water (50 mL). This resulted in 279d (65 g, 94.55%) as an off-white solid. The crude product was used in the next step directly without further purification.

### 5. Synthesis of 279e

To a stirred solution of NaOH (51.70 g, 1292.597 mmol, 8.00 equiv) in H2O (1.2 L) was added 279d (65 g, 161.567 mmol, 1 equiv) at room temperature. The resulting mixture was stirred overnight at room temperature. The residue was acidified to pH 5 with 1 mol/L HCl (aq.). The precipitated solids were collected by filtration and washed with water (50 mL). This resulted in 279e (50 g, 76.50%) as an off-white solid.

### 6. Synthesis of 279f

To a stirred solution of 279e (50 g, 130.110 mmol, 1 equiv) and HOAc (23.44 g, 390,330 mmol, 3 equiv) in DCM (500 mL) was added H2O2 (36.88 g, 325.275 mmol, 2.5 equiv, 30%) dropwise at 0° C. The resulting mixture was stirred overnight at room temperature. The mixture was basified to pH 10 with NaOH (l M). The aqueous layer was extracted with DCM (3×200 mL). The organic layers were concentrated under reduced pressure. The residue was purified by silica gel column chromatography, eluted with DCM/MeOH (100:1) to afford 279f (26 g, 53.90%) as a light yellow solid.

### 7. Synthesis of 279g

To a stirred solution of 279f (26 g, 73.815 mmol, 1 equiv) and THF (50 mL) in H2O (200 mL) was added HCl (100 mL, 4M) at room temperature. The resulting mixture was stirred overnight at 80° C. The mixture was neutralized to pH 8 with NaHCO₃ (aq.). The aqueous layer was extracted with EtOAc (3×100 mL). The resulting mixture was concentrated under vacuum. The residue was purified by reverse flash chromatography with the following conditions: column, C18 silica gel; mobile phase, MeCN in water (10 mmol/L NH4HCO3), 0% to 40% gradient in 20 min; detector, UV 220 nm. This resulted in 279g (10 g, 41.15%) as a white solid.

### 8. Synthesis of 279h

To a stirred solution of 279g (2 g, 6.532 mmol, 1 equiv) in DCM (50 mL) was added BAST (7.23 g, 32.660 mmol, 5 equiv) at 0° C. under nitrogen atmosphere. The resulting mixture was stirred for overnight at room temperature. The reaction was quenched with NaHCO3 (aq.) (200 mL) at room temperature. The aqueous layer was extracted with DCM (2×100 mL). The resulting mixture was concentrated under vacuum. The residue was purified by reverse flash chromatography with the following conditions: column, C18 silica gel; mobile phase, MeCN in water (10 mmol/L, NH4HCO3), 0% to 40% gradient in 20 nm; detector, UV 254 nm. This resulted in 279h (300 mg, 12.60%) as a yellow solid.

### 9. Synthesis of 279i

To a solution of 279h (300 mg, 0.914 mmol, 1 equiv) and Cu2O (26.16 mg, 0.183 mmol, 0.2 equiv) in MeCN (3 mL) was added NH4OH (3 mL) in a 10 mL pressure tank reactor. The resulting mixture was stirred overnight at 100° C. The mixture was allowed to cool down to room temperature. The resulting mixture was concentrated under vacuum. The residue was purified by Prep-TLC (DCM/MeOH 10:1) to afford 279i (100 mg, 37.25%) as a light yellow solid.

### 10. Synthesis of 279j

To a stirred solution of 279i (90 mg, 0.341 mmol, 1 equiv) and I-2 (127.62 mg, 0.443 mmol, 1.3 equiv) in DCE (5 mL) was added NaBH(OAc)3 (216.53 mg, 1.023 mmol, 3 equiv) at room temperature. The resulting mixture was stirred overnight at room temperature. The reaction was quenched with saturated NaHCO₃ (aq.) (20 mL) at room temperature. The aqueous layer was extracted with DCM (2×20 mL). The resulting mixture was concentrated under vacuum. The residue was purified by Prep-TLC (DCM/MeOH 20:1) to afford 279j (90 mg, 45.81%) as a light yellow solid.

### 11. Synthesis of 279

To a stirred solution of 279j (80 mg, 0.149 mmol, 1 equiv) and Pyridine (117.94 ng, 1.490 mmol, 10 equiv) in DCM (5 mL) was added Triphosgene (22.12 mg, 0.074 mmol, 0.5 equiv) at 0° C. The resulting mixture was stirred for 10 min at room temperature. The reaction was quenched with saturated NaHCO3 (aq.) (20 mL) at room temperature. The aqueous layer was extracted with DCM (2×20 mL). The resulting mixture was concentrated under vacuum. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 14% B to 25% B in 8 min; Wave Length: 254; 220 nm; RT1 (min): 7.98) to afford 279 (24.9 mg, 29.60%) as a yellow solid.

LC-MS: (ES, m/z): [M+H]+563. H-NMR: (400 MHz, CD30D, δ ppm): 1.14-1.15 (d, 3H), 1.88-1.93 (m, 1H), 2.19-2.24 (m, 1H), 2.76-2.85 (m, 2H), 3.37 (s, 2H), 3.42 (s, 3H), 3.45-3.49 (m, 2H), 3.64-3.75 (m, 4H), 3.85-3.88 (m, 1H), 7.11 (s, 1H), 7.19 (s, 1H), 7.36-7.38 (d, 1H), 7.57-7.61 (m, 1H), 7.66-7.70 (m, 2H), 7.79 (s, 1H), 8.42 (s, 1H).

### Example 280. Synthesis of Compound 280

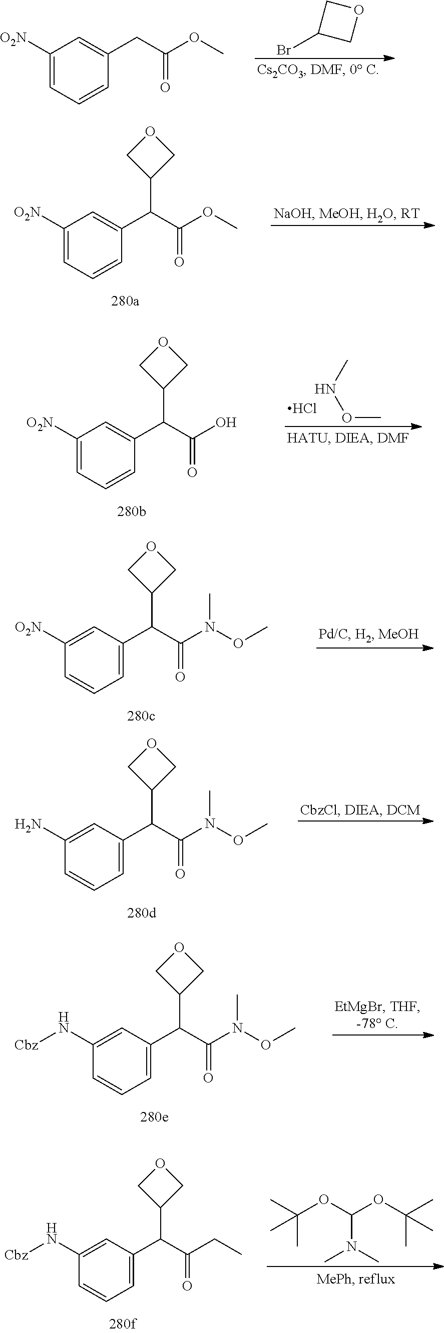

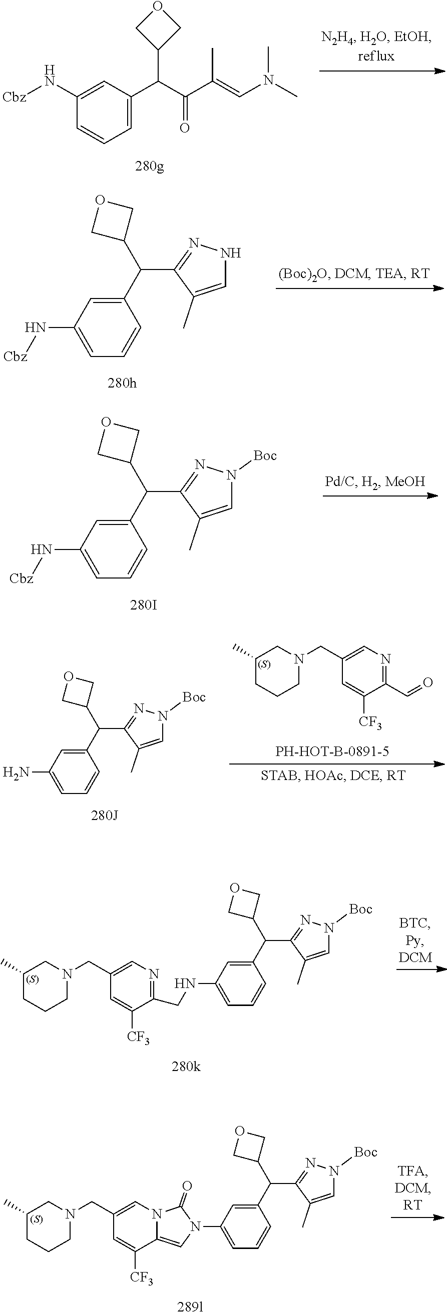

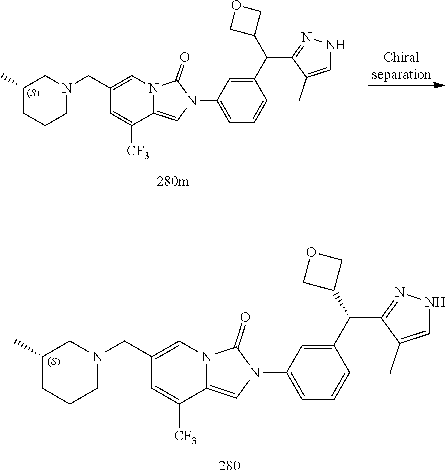

### 1. Synthesis of 280a

To a stirred solution of methyl 2-(3-nitrophenyl)acetate (48 g, 245.934 mmol, 1 equiv) in DMF (1.5 L) was added Cs2CO3 (400.65 g, 1229,670 mmol, 5 equiv) at 0° C. under nitrogen atmosphere. The resulting mixture was stirred for 2h at 0° C. under nitrogen atmosphere. To the above mixture was added 3-bromooxetane (101.06 g, 737.793 mmol, 3.00 equiv) at room temperature. The resulting mixture was stirred for additional 2 days at room temperature. The reaction was quenched with NH4Cl (aq.) (3 L) at room temperature. The aqueous layer was extracted with EtOAc (2×1 L). The organic layers were concentrated under reduced pressure. The residue was purified by silica gel column chromatography, eluted with PE/EA (50:1) to afford 280a (34 g, 49.52%) as an off-white solid.

### 2, Synthesis of 280b

To a stirred solution of 280a (34 g, 135,330 mmol, 1 equiv) in MeOH (100 mL) and H2O (300 mL) was added NaOH (16.24 g, 405,990 mmol, 3 equiv) at room temperature. The resulting mixture was stirred overnight at room temperature. The resulting mixture was diluted with water (500 mL). The aqueous layer was extracted with EtOAc (2×500 mL). The combined organic layers were dried over anhydrous Na2SO4. After filtration, the filtrate was concentrated under reduced pressure. This resulted in 280b (32 g, 89.71%) as an off-white solid.

### 3. Synthesis of 280c

To a stirred solution of 280b (19.74 g, 202.352 mmol, 1.5 equiv) in DMF (500 ml) were added HATU (66.68 g, 175,371 mmol, 1.3 equiv) and DIEA (52.31 g, 404.703 mmol, 3 equiv) at room temperature under nitrogen atmosphere. The resulting mixture was stirred for overnight at room temperature. The resulting mixture was diluted with water (1 L). The aqueous layer was extracted with EtOAc (2×500 mL). The residue was purified by silica gel column chromatography, eluted with PB/EA (3:1) to afford crude product. The residue was purified by reverse flash chromatography with the following conditions: column, C18 silica gel; mobile phase, MeCN in water, 10% to 50% gradient in 10 min; detector, LV 254 nm. This resulted in 280c (12 g, 29.52%) as an off-white solid.

### 4. Synthesis of 280d

To a solution of 280c (12 g, 42.814 mmol, 1 equiv) in MeOH (300 mL) was added Pd/C (1.2 g, 10%) in a 1000 mL round-bottom flask. The mixture was hydrogenated at room temperature for overnight under hydrogen atmosphere using a hydrogen balloon, filtered through a Celite pad and concentrated under reduced pressure. The resulting mixture was concentrated under vacuum. This resulted in 280d (12 g, 99.66%) as a white solid.

### 5. Synthesis of 280e

To a stirred solution of 280d (12 g, 47.943 mmol, 1 equiv) and CbzCl (16.36 g, 95.886 mmol, 2 equiv) in DCM (200 mL) was added DIEA (18.59 g, 143.829 mmol, 3 equiv) at room temperature under nitrogen atmosphere. The resulting mixture was stirred overnight at room temperature. The reaction was quenched with water at room temperature. The aqueous layer was extracted with DCM (2×500 mL). The combined organic layers were dried over anhydrous Na2SO4. After filtration, the filtrate was concentrated under reduced pressure. The residue was purified by silica gel column chromatography, eluted with PE/EA (2:1) to afford 280e (15 g, 73.25%) as an off-white solid.

### 6. Synthesis of 280f

To a stirred solution of 280e (14.9 g, 38.758 mmol, 1 equiv) in THF (150 mL) was added EtMgBr (193.79 mL, 387.580 mmol, 10 equiv, 2M) at −78° C. under nitrogen atmosphere. The resulting mixture was stirred for 6h at room temperature under nitrogen atmosphere. The reaction was quenched with saturated NH4Cl (aq.) (500 mL) at room temperature. The aqueous layer was extracted with EtOAc (2×200 mL). The organic layers were concentrated under reduced pressure. The residue was purified by silica gel column chromatography, eluted with PE/EA (5:1) to afford 280f (6 g, 39.86%) as an off-white solid.

### 7. Synthesis of 280g

To a stirred solution of 280f (6 g, 16.977 mmol, 1 equiv) in Toluene (100 mL) was added [bis(tert-butoxy)methyl]dimethylamine (10.36 g, 50.931 mmol, 3 equiv) at room temperature under nitrogen atmosphere. The resulting mixture was stirred overnight at 110° C. under nitrogen atmosphere. The resulting mixture was concentrated under vacuum. This resulted in 280g (7 g, 90.84%) as a light yellow oil. The crude product was used in the next step directly without further purification.

### 8. Synthesis of 280h

To a stirred solution of 280g (7 g, 17.136 mmol, 1 equiv) in EtOH (100 mL) was added hydrazine hydrate (98%)(8.58 g, 171.360 mmol, 10 equiv) at room temperature under nitrogen atmosphere. The resulting mixture was stirred overnight at 80° C. The reaction was quenched with water (300 mL) at room temperature. The aqueous layer was extracted with EtOAc (2×100 mL). The resulting mixture was concentrated under vacuum. The residue was purified by silica gel column chromatography, eluted with PE/EA (1:1) to afford 280h (1.4 g, 19.48%) as a white solid.

### 9. Synthesis of 280i

To a stirred solution of 280h (1.4 g, 3,709 mmol, 1 equiv) and (Boc)2O (1.62 g, 7.418 mmol, 2 equiv) in DCM (20 mL) was added TEA (1.13 g, 11.127 mmol, 3 equiv) at room temperature. The resulting mixture was stirred overnight at room temperature. The reaction was quenched with water (100 in) at room temperature. The aqueous layer was extracted with DCM (2×100 mL). The organic layers were concentrated under reduced pressure. The residue was purified by Prep-TLC (PE/EA 3:1) to afford 280i (1 g, 50.81%) as a yellow solid.

### 10. Synthesis of 280j

To a solution of 280i (1 g, 2.094 mmol, 1 equiv) in MeOH (30 mL) was added Pd/C (100 mg, 10%) under nitrogen atmosphere in a 250 mL round-bottom flask. The mixture was hydrogenated at room temperature for overnight under hydrogen atmosphere using a hydrogen balloon, filtered through a Celite pad and concentrated under reduced pressure. The resulting mixture was concentrated under vacuum. The crude product was used in the next step directly without further purification. This resulted in 280j (600 mg, 70.92%) as an off-white solid,

### 11. Synthesis of 280k

To a stirred solution of 289j (300 mg, 0.874 mmol, 1 equiv) and 1-2 (325.12 mg, 1.136 mmol, 1.3 equiv) in DCE (5 mL) was added STAB (370.28 mg, 1.748 mmol, 2 equiv) at room temperature under nitrogen atmosphere. The resulting mixture was stirred overnight at room temperature. The reaction was quenched with saturated NaHCO3 (aq.) (100 mL) at room temperature. The aqueous layer was extracted with DCM (2×20 mL). The organic layers were concentrated under reduced pressure. The residue was purified by Prep-TLC (DCM/MeOH 30:1) to afford 280k (250 mg, 42.43%) as a white solid.

### 12. Synthesis of 2801

To a stirred solution of 280k (240 mg, 0.391 mmol, 1 equiv) and Pyridine (309.32 mg, 3.910 mmol, 10 equiv) in DCM (200 mL) was added Triphosgene (58.02 mg, 0,196 mmol, 0.5 equiv) at room temperature under nitrogen atmosphere. The resulting mixture was stirred for 10 min at room temperature. The reaction was quenched with saturated NaHCO3 (aq.) (100 mL) at room temperature. The aqueous layer was extracted with DCM (2×l 0 mL). The organic layers were concentrated under reduced pressure. This resulted in 2801 (200 mg, 63.96%) as a yellow solid. The crude product was used in the next step directly without further purification.

### 13. Synthesis of 280m

To a stirred solution of 2891 (200 mg, 0.313 mmol, 1 equiv) in DCM (5 mL) was added TFA (2 mL) at room temperature. The resulting mixture was stirred for 3h at room temperature. The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 44% B to 72% B in 8 min, Wave Length: 220 nm; RT1 (min): 7.58) to afford 280m (100 mg, 58.09%) as a yellow solid.

### 14. Synthesis of 280

280m (100 mg) was purified by Prep-Chiral-HPLC with the following conditions (Column: CHIRALPAK OD-H, 2*25 cm, 5 μm; Mobile Phase A: Hex (0.5% 2M NH3-MeOH), Mobile Phase B: EtOH; Flow rate: 20 mL/min; Gradient: 20% B to 20% B in 12 min; Wave Length: 220/254 nm; RT1 (min): 8.33; RT2 (min): 10.54; the first peak is product) to afford the crude product. The crude product (22 mg) was purified by Prep-AChiral-SFC with the following conditions (Column: DAICEL DCpak P4VP, 2*25 cm, 5 μm; Mobile Phase A: CO2, Mobile Phase B: ACN: MEOH=4:1 (0.1% 2M NH3-MEOH); Flow rate: 50 mL/min; Gradient: isocratic 20% B; Column Temperature (° C.): 35; Back Pressure (bar): 100; Wave Length: 254 nm; RT1 (min): 3.62) to afford 280 (8.4 mg, 8.37%) as a yellow solid,

LC-MS 0: (ES, m/z): [M+H]+ 540. H-NMR: (400 MHz, CD30D, δ ppm): 0.85-1.02 (m, 4H), 1.61-1.84 (m, 8H), 1.99-2.09 (m, 1H), 2.88-2.94 (m, 2H), 3.06-3.11 (m, 1H), 3.38 (s, 2H), 3.76-3.82 (m, 2H), 4.02-4.06 (m, 1H), 4.33-4.34 (m, 1H), 4.38-4.40 (m, 1H), 7.13 (s, 1H), 7.14-7.16 (d, 1H), 7.25-7.26 (d, 1H), 7.35 (s, 1H), 7.52-7.56 (m, 1H), 7.65-7.66 (m, 2H), 7.69 (s, 1H).

### Example 281. Synthesis of Compound 281

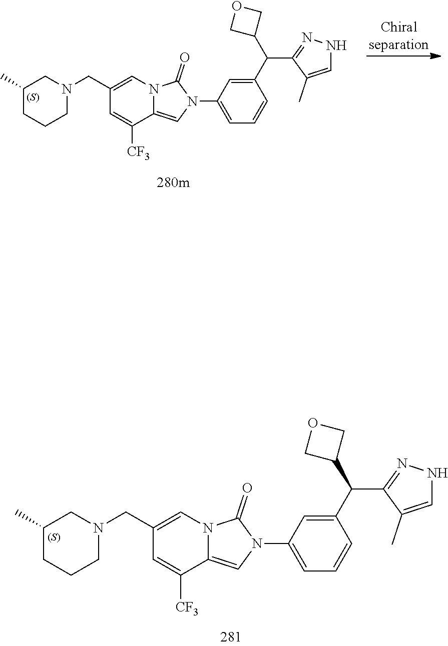

### Synthesis of 281

280m (100 mg) was purified by Prep-Chiral-HPLC with the following conditions (Column: CHIRALPAK OD-H, 2*25 cm, 5 μm; Mobile Phase A: Hex (0.5% 2M NH3-MeOH), Mobile Phase B: EtOH; Flow rate: 20 mL/min; Gradient: 20% B to 20% B in 12 min; Wave Length: 220/254 nm; RT1 (min): 8.33; RT2 (min): 10.54; the second peak is product) to afford 281 (29.5 mg, 29.26%) as a yellow solid,

LC-MS: (ES, m/z): [M+H]+ 540. H-NMR: (400 MHz, CD30D, δ ppm): 0.96-1.01 (d, 3H), 1.06-1.09 (m, 1H), 1.61-1.84 (m, 7H), 2.10-2.21 (m, 1H), 2.36-2.51 (m, 1H), 3.06-3.32 (m, 3H), 3.74-3.84 (m, 4H), 4.02-4.06 (m, 1H), 4.33-4.38 (d, 1H), 4.38-4.42 (m, 1H), 7.13 (s, 1H), 7.21-7.23 (d, 1H), 7.26-7.28 (d, 1H), 7.34 (s, 1H), 7.52-7.56 (m, 1H), 7.65-7.66 (m, 2H), 7.84 (s, 1H).

### Example 282. Synthesis of Compound 282

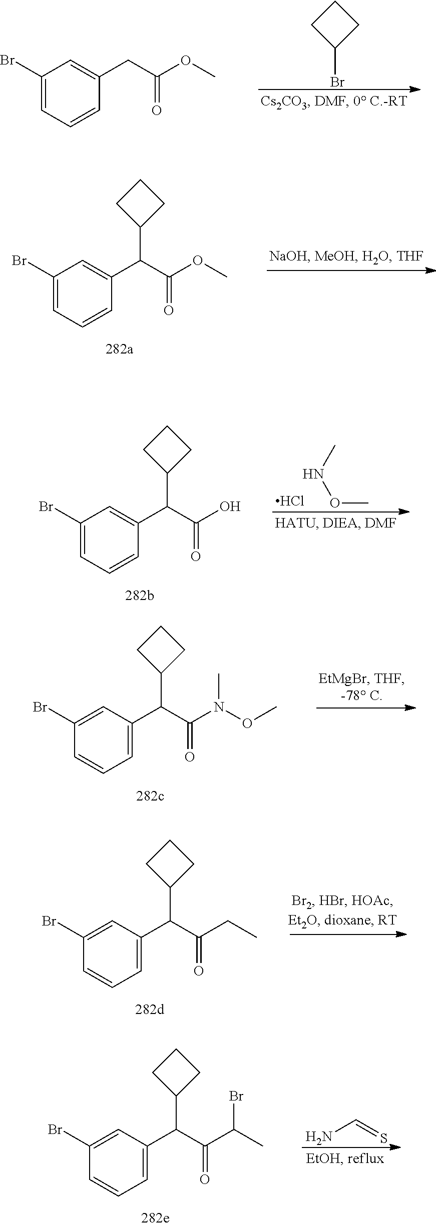

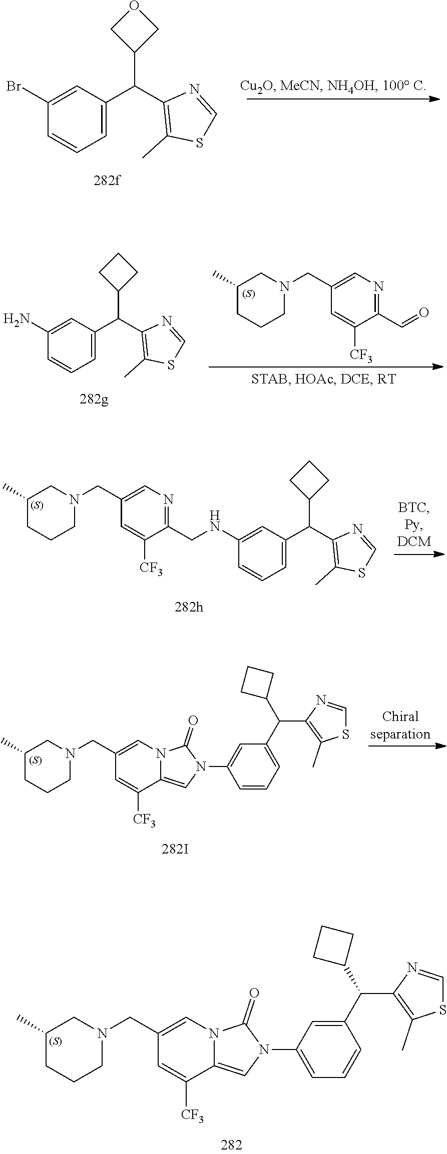

### 1. Synthesis of 282a

To a stirred mixture of methyl 2-(3-bromophenyl)acetate (50 g, 218.271 mmol, 1 equiv) in DMF (500 mL) was added Cs2CO3 (355.58 g, 1091.355 mmol, 5 equiv) at 0° C. under nitrogen atmosphere. The resulting mixture was stirred for 3 h at room temperature under nitrogen atmosphere. To the above mixture was added bromocyclobutane (88.40 g, 654.813 mmol, 3 equiv) at room temperature. The resulting mixture was stirred for additional 24 h at room temperature. The resulting mixture was diluted with water (3000 mL). The aqueous layer was extracted with EtOAc (2×1000 mL). The resulting mixture was concentrated under reduced pressure. The residue was purified by silica gel column chromatography, eluted with PE/EA=50:1 to afford 282a (27 g, 43.68%) as a white solid.

### 2. Synthesis of 282b

To a stirred mixture of 282a (48 g, 169.512 mmol, 1 equiv) in MeOH (600 mL) was added NaOH (20.34 g, 508,536 mmol, 3 equiv) in H2O (200 mL) at room temperature. The mixture was stirred for 2 h at room temperature. The mixture was neutralized to pH 6 with HCl (1 M). The aqueous layer was extracted with EtOAc (2 L). The resulting mixture was concentrated under reduced pressure. This resulted in 282b (50 g, 98.64%) as a yellow oil.

### 3. Synthesis of 282c

To a stirred mixture of 282b (50 g, 185,778 mmol, 1 equiv) and methoxy(methyl)amine hydrochloride (36.24 g, 371.556 mmol, 2 equiv) in DMF (550 mL) was added HATU (77.70 g, 204.356 mmol, 1.1 equiv) and DIEA (72.03 g, 557.334 mmol, 3 equiv) at room temperature. The mixture was stirred for 2 b at room temperature. The reaction was quenched with water (1500 mL) at room temperature. The aqueous layer was extracted with EtOAc (1 L). The resulting mixture was concentrated under reduced pressure. The residue was purified by silica gel column chromatography, eluted with PE/EA=12:1 to afford 282c (50 g, 77.58%) as a yellow oil,

### 4, Synthesis of 282d

To a stirred mixture of 282c (8 g, 25.624 mmol, 1 equiv) in THF (100 mL) was treated with EtMgBr (64,060 mL, 128.120 mmol, 5 equiv, 2 M in THE) at −78° C. under nitrogen atmosphere. The mixture was stirred for 2 h at −78° C. The reaction was quenched by the addition of NH4Cl (aq.) (1000 mL) at room temperature. The aqueous layer was extracted with EA (500 mL). The resulting mixture was concentrated under reduced pressure. The residue was purified by silica gel column chromatography, eluted with PE/EA=5/1 to afford 282d (2.8 g, 34.97%) as a yellow oil.

### 5. Synthesis of 282e

To a stirred solution of 282d (2.4 g, 8.535 mmol, 1 equiv) in dioxane (24 ml) and Et2O (24 ml) was added HBr in AcOH (0.48 g, 5.974 mmol, 0.7 equiv, 40%) and Br2 (0.95 g, 5.974 mmol, 0.7 equiv) at 0° C. The resulting mixture was stirred for 2 h at room temperature. The reaction was quenched by the addition of NaHCO₃ (aq.) (200 mL) at room temperature. The aqueous layer was extracted with EtOAc (100 mL). The resulting mixture was concentrated under reduced pressure. The residue was purified by silica gel column chromatography, eluted with PE/EA-50:1 to afford 282e (1 g, 28.31%) as a colorless oil.

### 6. Synthesis of 282f

To a stirred solution of 282e (1.08 g, 2,999 mmol, 1 equiv) in EtOH (10 mL) was added thioformamide (0.27 g, 4,498 mmol, 1.5 equiv) at room temperature. The resulting mixture was stirred for overnight at 80° C. The mixture was allowed to cool down to room temperature. The resulting mixture was concentrated under reduced pressure. The residue was purified by silica gel column chromatography, eluted with PE/EA=40:1 to afford 282f (4.2 g, 434.54%) as a yellow oil.

### 7. Synthesis of 282g

To a stirred mixture of 282g (400 mg, 1,241 mmol, 1 equiv) in MeCN (2 mL) and NH40H (2 mL) were added Cu2O (35.52 mg, 0.248 mmol, 0.2 equiv) at room temperature. The resulting mixture was stirred for overnight at 100° C. The mixture was allowed to cool down to room temperature. The resulting mixture was concentrated under reduced pressure. The residue was purified by Prep-TLC (PE/EA=5:1) to afford 282g (220 mg, 68.60%) as a yellow solid.

### 8. Synthesis of 282h

To a stirred mixture 282g (200 mg, 0.774 mmol, 1.00 equiv) and I-2 (221.61 mg, 0.774 mmol, 1.00 equiv) in DCE (2 mL) were added HOAc (46.48 mg, 0.774 mmol, 1 equiv) and STAB (492.16 mg, 2.322 mmol, 3 equiv) at room temperature. The resulting mixture was stirred for 2 h at room temperature. The reaction was quenched by the addition of NaHCO₃ (aq.) (20 mL) at room temperature. The aqueous layer was extracted with EtOAc (2×10 mL).The resulting mixture was concentrated under reduced pressure. The residue was purified by Prep-TLC (CH2Cl2/MeOH=40:1) to afford 282h (240 mg, 53.37%) as a yellow solid.

### 9. Synthesis of 282i

To a stirred solution of 282h (220 mg, 0,416 mmol, 1 equiv) and Pyridine (197.50 mg, 2,496 mmol, 6 equiv) in DCM (5 mL) was added Triphosgene (43.22 mg, 0.146 mmol, 0.35 equiv) at 0° C. The resulting mixture was stirred for 30 min at room temperature. The reaction was quenched by the addition of NaHCO₃ (aq.) (30 ml) at room temperature. The aqueous layer was extracted with CH2Cl2 (15 mL). The resulting mixture was concentrated under reduced pressure. The residue was purified by Prep-TLC (CH2Cl2/MeOH=15:1) to afford the crude product (200 mg). The residue was purified by reverse flash chromatography with the following conditions: column, C18; mobile phase, MeCN in water (0.1% NH4HCO3) 2% to 100% gradient in 30 min; detector, UV 254 nm to afford 282i (140 mg, 60.05%) as a yellow solid,

### 10. Synthesis of 282

282i (140 mg) was separated by Prep-CHIRAL-HPLC with the following conditions (Column: CHIRALPAK IE, 2*25 cm, 5 μm; Mobile Phase A: Hex (0.5% 2M NH3-MeOH), Mobile Phase B: EtOH:DCM=1:1; Flow rate: 20 m/min; Gradient: 10% B to 10% B in 16 min; Wave Length: 220/254 nm; RT1 (min): 10.47; RT2 (min): 1309; the first peak is product) to afford 282 (48.7 mg, 34.44%) as a yellow solid.

LCMS: (ES, m/z): [M+H]+ 555. H-NMR: (400 MHz, DMSO-d6, ppm, δ): 0.75-0.92 (m, 4H), 1.49-1.58 (m, 1H), 1.66-1.76 (m, 5H), 1.76-1.88 (m, 6H), 2.46 (s, 3H), 2.75 (s, 2H), 3.24-3.27 (m, 3H), 4.15-4.18 (d, 1H), 7.00 (s, 1H), 7.28 (s, 1H), 7.35-7.41 (m, 2H), 7.58-7.66 (m, 2H), 7.83 (s, 1H), 8.80 (s, 1H).

### Example 283. Synthesis of Compound 283

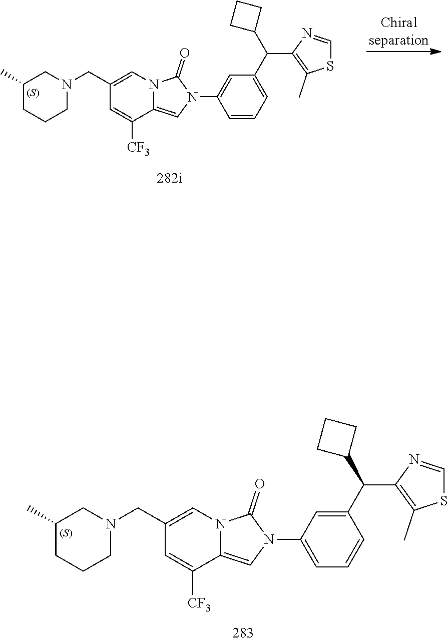

### Synthesis of 283

282i (140 mg) was separated by Prep-Chiral-HPLC with the following conditions (Column: CHIRALPAK IE, 2*25 cm, 5 μm; Mobile Phase A: Hex (0.5% 2M NH3-MeOH), Mobile Phase B: EtOH:DCM=1:1; Flow rate: 20 mL/min; Gradient: 10% B to 10% B in 16 min; Wave Length: 220/254 am; RT1 (min): 10.47; RT2 (mini): 13.09; the second peak is product) to afford 283 (49.4 mg, 36.44%) as a yellow solid.

LCMS: (ES, m/z): [M+H]+ 555. H-NMR: (400 MHz, DMSO-d6, ppm, δ): 0.75-0.92 (m, 4H), 1.49-1.58 (m, 1H), 1.66-1.76 (m, 5H), 1.77-1.88 (m, 6H), 2.46 (s, 3H), 2.75 (s, 2H), 3.24-3.27 (m, 3H), 4.15-4.18 (d, 1H), 7.00 (s, 1H), 7.28 (s, 1H), 7.35-7.41 (m, 2H), 7.58-7.66 (m, 2H), 7.83 (s, 1H), 8.80 (s, 1H).

### Example 284. Synthesis of Compound 284

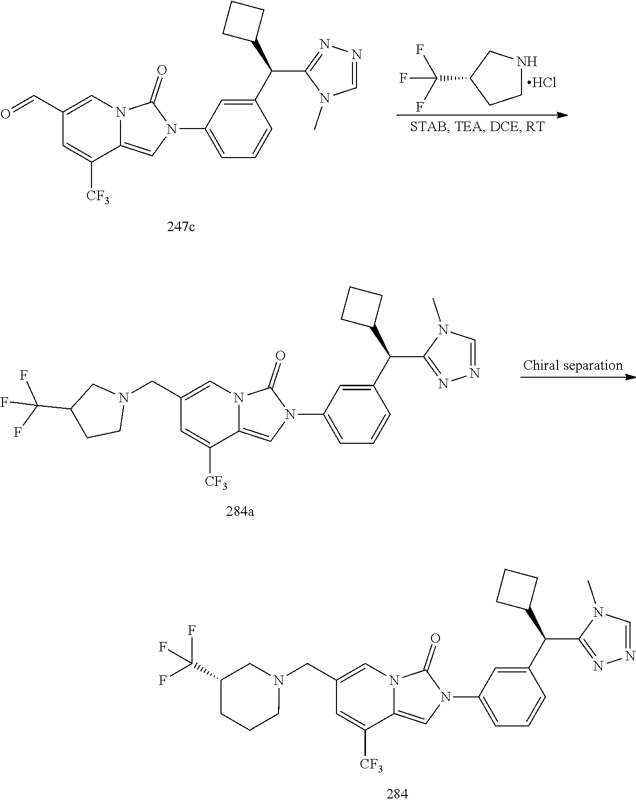 [SMILES: Cn1cnnc1[C@@H](c1cccc(-n2cc3c(C(F)(F)F)cc(C=O)cn3c2=O)c1)C1CCC1 ; FC(F)(F)[C@H]1CCNC1 ; Cl ; Cn1cnnc1[C@@H](c1cccc(-n2cc3c(C(F)(F)F)cc(CN4CCC(C(F)(F)F)C4)cn3c2=O)c1)C1CCC1 ; Cn1cnnc1[C@@H](c1cccc(-n2cc3c(C(F)(F)F)cc(CN4CCC[C@H](C(F)(F)F)C4)cn3c2=O)c1)C1CCC1]

### Synthesis of 284a

To a stirred solution of 247c (500 mg, 1.098 mmol, 1.00 equiv) and (3S)-3-(trifluoromethyl)pyrrolidine hydrochloride (385.52 mg, 2.196 mmol, 2 equiv) in DCE (6 mL) were added TEA (222.19 mg, 2.196 mmol, 2 equiv) at room temperature. The mixture was stirred for 30 min at room temperature. To the above mixture was added STAB (465.35 mg, 2.196 mmol, 2 equiv) at room temperature. The resulting mixture was stirred for overnight at room temperature. The reaction was quenched by the addition of sat. NH4Cl (aq.) (15 mL) at room temperature. The resulting mixture was extracted with CH2Cl2 (3×30 mL). The resulting mixture was concentrated under reduced pressure. The crude product (300 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 39% B to 69% B in 8 min, 69% B; Wave Length: 220 nm; RT1 (min): 7.18) to afford 284a (210 mg, 32.07%) as a yellow solid.

### Example 285. Synthesis of Compound 285

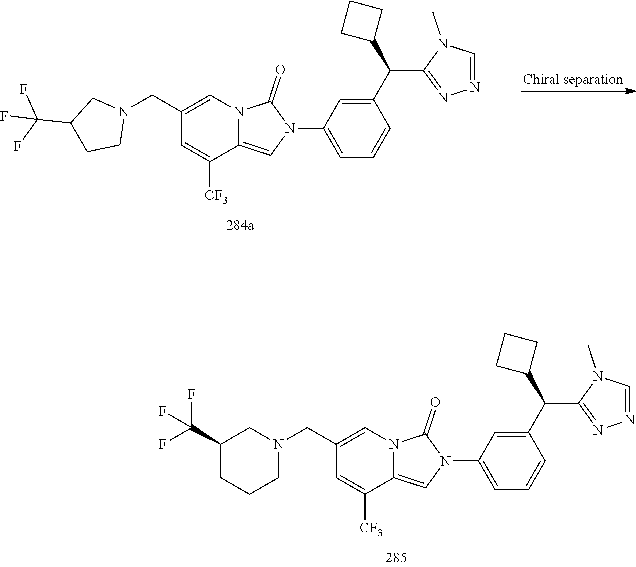 [SMILES: Cn1cnnc1[C@@H](c1cccc(-n2cc3c(C(F)(F)F)cc(CN4CCC(C(F)(F)F)C4)cn3c2=O)c1)C1CCC1 ; Cn1cnnc1[C@@H](c1cccc(-n2cc3c(C(F)(F)F)cc(CN4CCC[C@@H](C(F)(F)F)C4)cn3c2=O)c1)C1CCC1]

### Synthesis of 285

284a (210 mg) was purified by Chiral separation with the following conditions (Column: CHIRALPAK IF, 2*25 cm, 5 μm; Mobile Phase A: MtBE (0.5% 2M NH3-MeOH)-HPLC, Mobile Phase B: MTBE: MEOH=1:1-HPLC; Flow rate: 20 mL/min; Gradient: 10% B to 10% B in 33 min; Wave Length: 220/254 nm; RT1 (min): 24.76; RT2 (min): 28.08; The second peak was the product. Sample Solvent: MTBE: MEOH=1:1-HPLC; Injection Volume: 0.2 mL; Number of Runs: 16). This resulted in 285 (73.3 mg, 34.77%) as a yellow solid.

LC-MS: (ES, m/z): [M+H]+: 579. H-NMR: (400 MHz, DMSO-d6, ppm): δ 1.60-1.82 (m, 6H) S 1.93-2.15 (m, 2H), δ 2.51-2.53 (d, 2H), δ 2.71-2.73 (m, 1H), δ 3.16-3.18 (m, 1H), δ 3.21-3.23 (m, 1H), δ 3.42-3.50 (m, 6H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 3H), δ 8.35 (s, 1H).

### Example 286. Synthesis of Compound 286

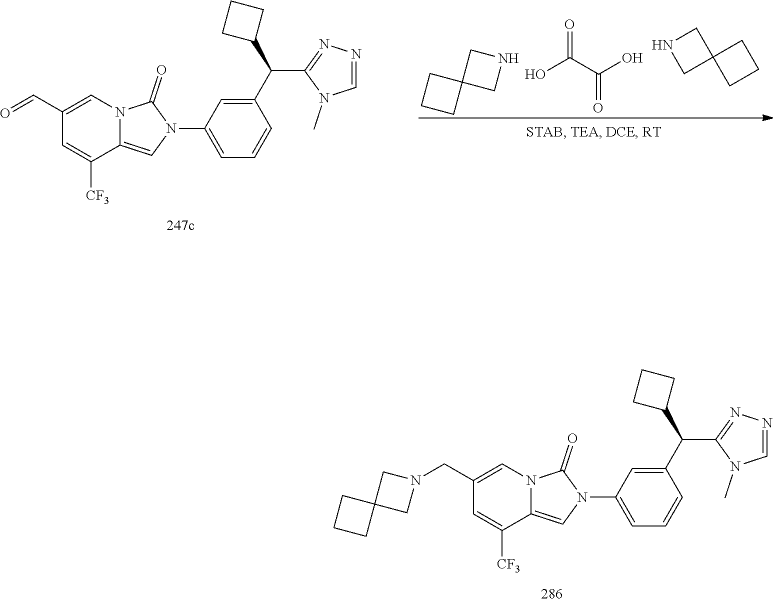

### Synthesis of 286

To a stirred solution of 247c (300 mg, 0.659 mmol, 1 equiv) and 2-azaspiro[3,3]heptane hemioxalate (374.14 mg, 1.318 mmol, 2 equiv) in DCE (5 m-L) was added TEA (133.31 mg, 1.318 mmol, 2 equiv) at room temperature. The resulting mixture was stirred for 1h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The organic layers were concentrated under reduced pressure. The residue was purified by Prep-TLC (DCM/MeOH 20:1) to afford the crude product. The residue was purified by reverse flash chromatography with the following conditions: column, C18 silica gel; mobile phase A: water (10 mmol/L NH4HCO3), B: MeCN, 10 B % to 55% B gradient in 20 min; detector, UV 254 nm. This resulted in 286 (166.3 mg, 46.86%) as a yellow solid.

LC-MS: (ES, m/z): [M+H]+ 537. H-NMR: (400 MHz, DMSO-d6, δ ppm): 1.63-1.93 (m, 7H), 2.03-2.15 (m, 5H), 3.13 (s, 4H), 3.17-3.30 (m, 1H), 3.32 (s, 2H), 3.43 (s, 3H), 4.25-4.27 (d, 1H), 6.95 (s, 1H), 7.18-7.20 (d, 1H), 7.31 (s, 1H), 7.42-7.46 (m, 1H), 7.62 (s, 1H), 7.68-7.70 (m, 1H), 7.74 (s, 1H), 8.33 (s, 1H).

### Example 287. Synthesis of Compound 287_P1 & 287_P2

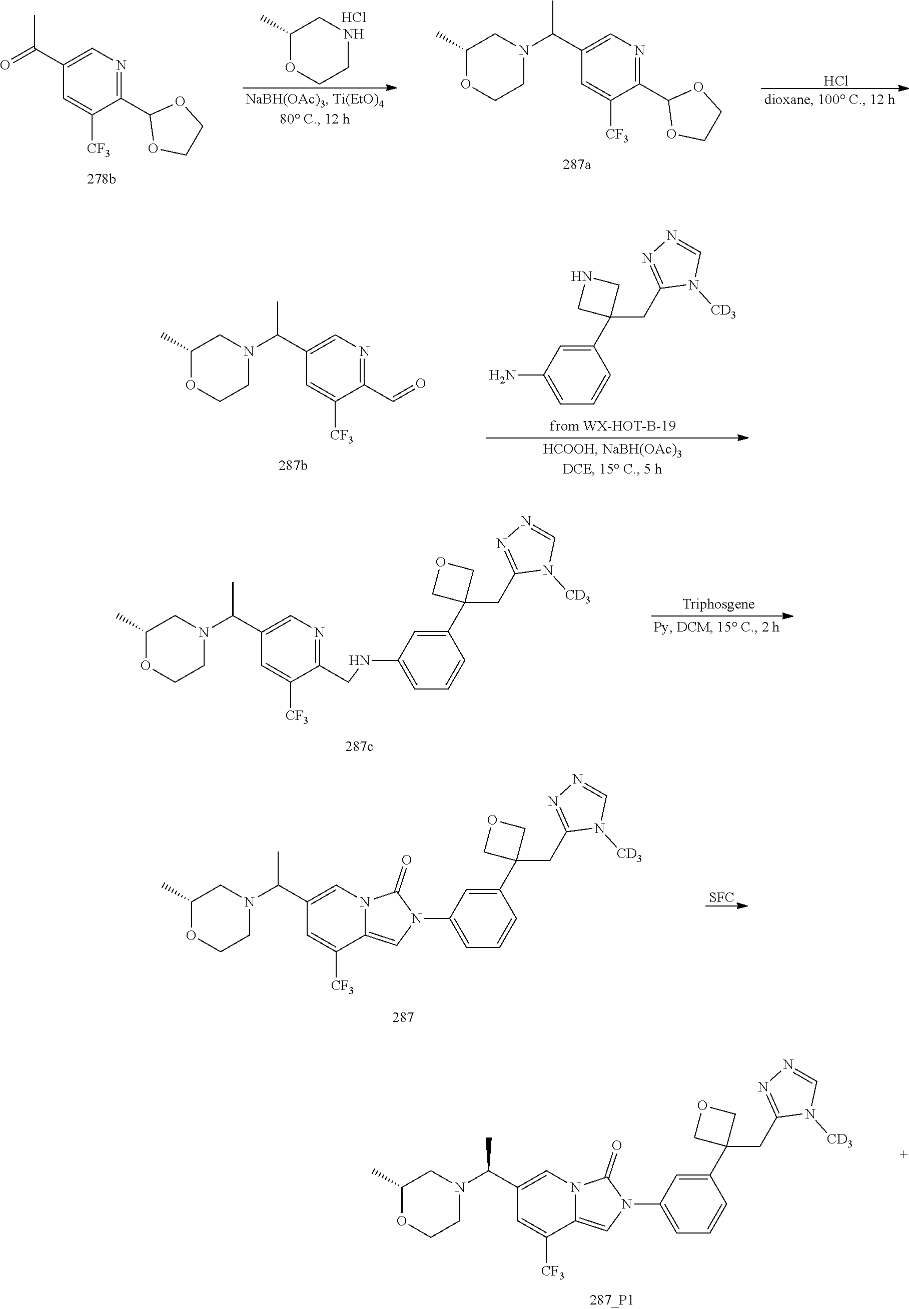

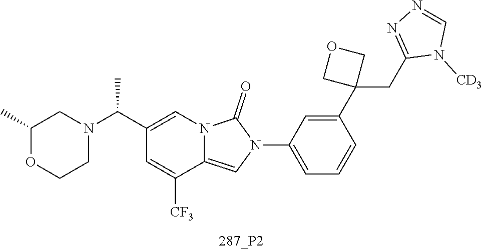 [SMILES: [2H]C([2H])([2H])n1cnnc1CC1(c2cccc(-n3cc4c(C(F)(F)F)cc([C@@H](C)N5CCO[C@H](C)C5)cn4c3=O)c2)COC1]

### 1. Synthesis of 287a

A mixture of 278b (16 g, 6.13 mmol, 1 equiv), (2R)-2-methylmorpholine (929.38 mg, 9.19 mmol, 1.5 equiv), Ti(i-PrO)4 (6.96 g, 24.50 mmol, 7:23 mL, 4 equiv) in DCE (20 mL) was degassed and purged with N2 for 3 times at 80° C. for 6 hr. Then it was added NaBH(OAc)3 (3.89 g, 18.38 mmol, 3 equiv) at r.t. The mixture was stirred at 80° C. for 6 hr under N2 atmosphere. The reaction mixture was quenched by addition Na2CO3 aq. 20 mL and diluted with H2O 50 mL, It was extracted with DCM (3*200 mL). The combined organic layers were dried over anhydrous sodium sulfate, filtered and the filtrate was concentrated under reduced pressure to give a residue. The residue was purified by column chromatography (SiO2, Petroleum ether/Ethyl acetate=50/1 to 0/1) to give 287a (600 mg, 1.73 mmol, 28.28% yield) as a white solid.

### 2. Synthesis of 287b

A mixture of 287a (1 g, 2.90 mmol, 1 equiv) in dioxane (4 mL) and HCl (4 ml, 4M) was degassed and purged with N2 for 3 times, and then the mixture was stirred at 100° C. for 6 hr under N2 atmosphere. The reaction mixture was quenched by addition Na2C03 aq. 20 ml and extracted with DCM (3*200 mL). The combined organic layers were dried over anhydrous sodium sulfate, filtered and the filtrate was concentrated under reduced pressure to give a residue. The crude product 287b (550 mg, 1.83 mmol, 63.07% yield) was obtained as yellow oil and used into the next step without further purification.

### 3. Synthesis of 287c

To a mixture of 287b, 3-[3-[[4-(trideuteriomethyl)-1,2,4-triazol-3-yl]methyl]azetidin-3-yl]aniline (285.20 mg, 1.16 mmol) in DCE (10 mL) and HCOOH (0.01 mL) was added NaBH(OAc)3 (245.39 mg, 1.16 mmol) and the mixture was stirred at r.t. for 5 hr under N2 atmosphere. The reaction mixture was quenched by addition Na2CO3 aq. 20 mL, and then diluted with H2O 20 mL. The mixture was extracted with DCM (3*50 mL). The combined organic layers were dried over anhydrous sodium sulfate, filtered and the filtrate was concentrated under reduced pressure to give a residue. The residue was purified by prep-HPLC (column: Waters Xbridge Prep OBD C18 150*40 mm*. 10 um; mobile phase: [water (NH4HCO3)-ACN]; B %: 30%-60%, 8 min). 287c (200 mg, 374.81 umol, 32.37% yield) was obtained as a white solid.

### 4. Synthesis of 287

A mixture of 287c (200 mg, 374.81 umol, 1 equiv), Py. (148.24 mg, 1.87 mmol, 5 equiv) in DCM (10 mL) was added TRIPHOSGENE (122.35 mg, 412.29 umol, 1.1 equiv) under N2. The mixture was stirred at 0° C. for 2 hr under N2 atmosphere. The reaction mixture was quenched by addition aq. NaHCO3 50 mL at 0° C. under N2. Then the mixture was extracted with DCM (3*50 mL). The combined organic layers were dried over anhydrous sodium sulfate, filtered and the filtrate was concentrated under reduced pressure to give a residue The residue was purified by prep-HPLC (column: Waters Xbridge BEH C18 100*30 mm*10 um; mobile phase: [water (NH4HCO3)-ACN]; B %: 35%-55%, 8 min). 287 (95 mg, 169.77 umol) was obtained as a yellow solid.

H-NMR: (400 MHz, DMSO-d6) δ 8.20 (s, 1H), 7.76-7.74 (m, 1H), 7.62 (s, 1H), 7.41 (d, J=8.4 Hz, 2H), 7.31 (s, 1H), 7.06 (s, 1H), 6.90 (d, 7.6 Hz, 1H), 4.96-4.91 (m, 41), 3.71-3.50 (m, 1H), 3.47-3.42 (m, 5H), 2.80-2.77 (m, 1H), 2.76-2.72 (m, 1H), 2.10-2.06 (m, 1 H), 1.79-1.74 (m, 1H), 1.27-1.25 (m, 3H), 1.07-1.01 (m, 1H).

### 5. Synthesis of 287_P1 & P2

287 (95 mg, 169.77 umol) was separated by SFC (column: Phenomenex-Cellulose-2 (250 mm*30 mm, 10 um); mobile phase: [0.1% NH3H2O MEOH]; B %: 65%-65%, 10 min) (column: DAICEL CHIRALCEL OD (250 mm*30 mm, 10 um); Mobile Phase: [0.1% NH3·H2O IPA]; B %: 37%-37%, 10 min, Flow rate: 70 mL/min; Wave Length: 220/254 nm; RT1 (min): 4.71; RT2 (min): 6.03) to get the two targets. 287_P1 (30 mg, 53.61 umol, 31.58% yield) was obtained as a yellow solid. 287 P2 (31 mg, 55.40 umol, 32.63% yield) was obtained as a yellow solid.

LCMS-287_P1: (ES, m/z): [M+H]+ 560.3. H-NMR-287_P1: (400 MHz, DMSO-d6) δ 8.21 (s, 1H), 7.66-7.63 (m, 2H), 7.48 (t, J=7.6 Hz, 1H), 7.31 (s, 1H), 7.16 (s, 1H), 7.12 (s, 1H), 6.92 (d, J=7.6 Hz, 1H), 5.09 (s, 4H), 3.90 (d, J=1.6 Hz, 1H), 3.70-3.60 (m, 5H), 2.86 (d, J=11.2 Hz, 1H), 2.74 (d, J=11.6 Hz, 1H), 2.22 (t, 8.4 Hz, 1H), 1.92 (t, J=10.8 Hz, 1H), 1.37 (d, J=6.4 Hz, 3H), 1.11 (d, J=6.4 Hz, 3H)

LCMS-287_P2: (ES, m/z): [M+H]+ 560.3. H-NMR-287_P2: (400 MHz, DMSO-d6) δ 8.21 (s, 1H), 7.66-7.63 (m, 2H), 7.48 (t, J=8 Hz, 1H), 7.31 (s, 1H), 7.16 (s, 1H), 7.11 (s, 1H), 6.91 (d, J=7.6 Hz, 1H), 5.09 (s, 4H), 3.82 (d, J=1.6 Hz, 1H), 3.68-3.61 (m, 5H), 2.92 (d, J=11.2 Hz, 1H), 2.67 (d, J=11.6 Hz, 1H), 2.26 (t, 8.4 Hz, 1H), 1.92 (t, J=10.8 Hz, 1H), 1.37 (d, J=6.4 Hz, 3H), 1.12 (d, J=6.4 Hz, 3H).

### Example 288. Synthesis of Compound 288

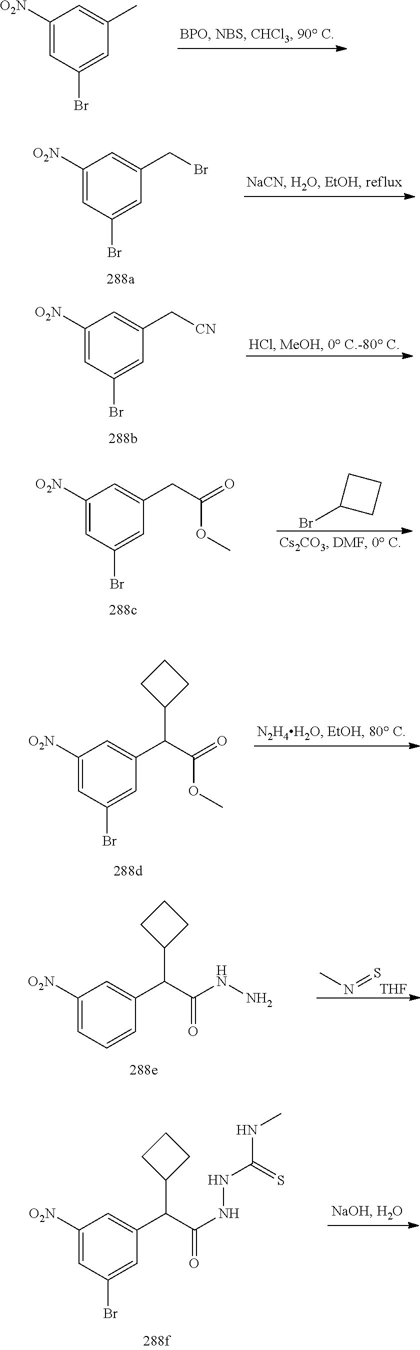

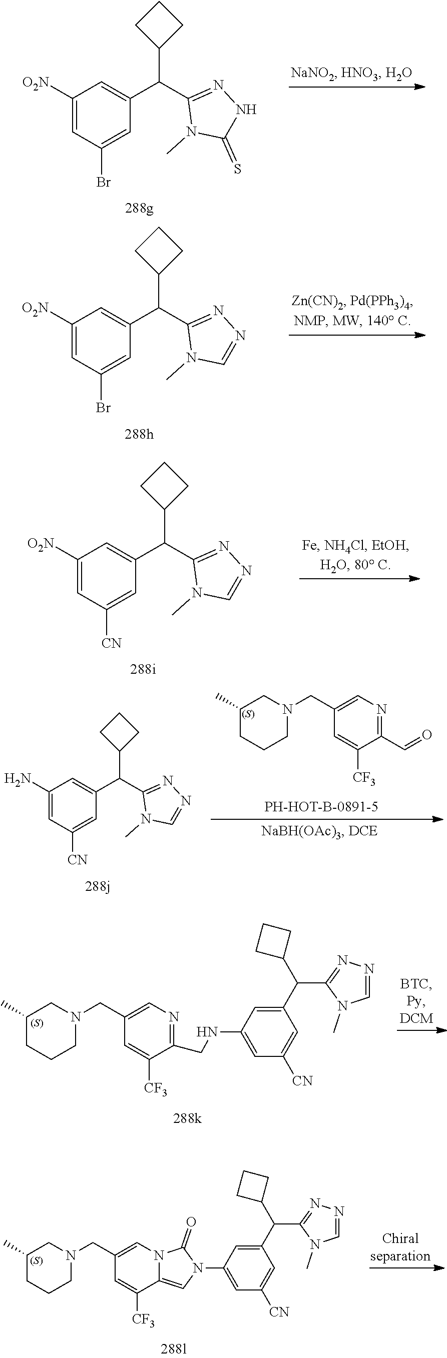

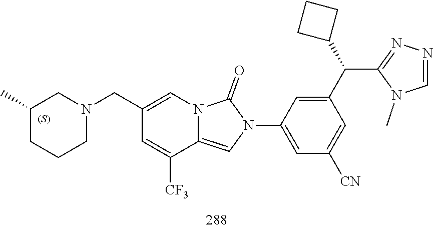

### 1. Synthesis of 288a

Into a 1 L 3-necked round-bottom flask was added 1-bromo-3-methyl-5-nitrobenzene (50 g, 231.445 mmol, 1 equiv) 28 (1186 g, 46.289 mmol, 0.2 equiv), NBS (8239 g, 462.890 mmol, 2 equiv) and CHCl3 (500 mL) at room temperature under nitrogen atmosphere. The resulting mixture was stirred overnight at 90° C. under nitrogen atmosphere. The reaction was quenched with NaHCO3 (aq.) (800 mL) at room temperature. The aqueous layer was extracted with EtOAc. (3×500 mL). The resulting mixture was concentrated under reduced pressure. The residue was purified by silica gel column chromatography, eluted with (PE/BA 10:1) to afford 288a (22 g, 34.08%) as an off-white solid.

### 2. Synthesis of 288b

To a stirred mixture of 288a (22 g, 74.594 mmol, 1 equiv) in EtOH (100 mL)/1120 (25 mL) was added at NaCN (5.48 g, 111.891 mmol, 1.5 equiv) 0° C. under nitrogen atmosphere. The resulting mixture was stirred for 1h at 80′C under nitrogen atmosphere. The mixture was basified to pH 8 with saturated NaHCO3 (aq.). The resulting mixture was extracted with EtOAc (3×100 mL). The combined organic layers were dried over anhydrous Na2SO4. After filtration, the filtrate was concentrated under reduced pressure. The residue was purified by silica gel column chromatography, eluted with PE/EA (10:1) to afford 288b (3 g, 15.85%) as an off-white solid.

### 3. Synthesis of 288c

Into a 250 mL 3-necked round-bottom flask were added 288b (3 g, 12.446 mmol, 1 equiv) and MeOH (30 mL) at 0° C. The resulting mixture was stirred for 2h at 0° C. under HCl(g). Then the resulting mixture was stirred for 2h at 80° C. under nitrogen atmosphere. The mixture was basified to pH 7 with saturated NaHCO3 (aq.).The aqueous layer was extracted with EtOAc (3×40 mL). The resulting mixture was concentrated under reduced pressure. The crude product was used in the next step directly without further purification. This resulted in 288c (2.4 g, 66.84%) as a light yellow solid.

### 4. Synthesis of 288d

To a stirred mixture of 288c (2.5 g, 9.122 mmol, 1 equiv) in DMF (30 mL) was added Cs2CO3 (14.86 g, 45.610 mmol, 5 equiv) at 0° C. under nitrogen atmosphere. The resulting mixture was stirred for 2h at 0° C. under nitrogen atmosphere. To the above mixture was added bromocyclobutane (3.69 g, 27,366 mmol, 3 equiv) at 0° C. The resulting mixture was stirred for additional overnight at room temperature. The mixture was acidified to pH 7 with saturated NH4Cl (aq.) (100 mL). The resulting mixture was extracted with EtOAc (3×100 mL). The combined organic layers were washed with water (2×30 mL), dried over anhydrous Na2SO4. After filtration, the filtrate was concentrated under reduced pressure. The residue was purified by silica gel column chromatography, eluted with PE/EA (50:1) to afford 288d (1.3 g, 41.26%) as a light yellow oil.

### 5. Synthesis of 288e

Into a 50 mL 3-necked round-bottom flask were added 288d (1.3 g, 3.961 mmol, 1 equiv), NH2NH2·H2O (1.98 g, 39.610 mmol, 10 equiv) and EtOH (15 mL) at room temperature. The resulting mixture was stirred overnight at 80° C. under nitrogen atmosphere. The mixture was allowed to cool down to room temperature. The reaction was quenched with water (100 mL) at room temperature. The aqueous layer was extracted with EtOAc (3×30 mL). The combined organic layers were concentrated under vacuum. This resulted in 288e (1.2 g, 84.00%) as a light yellow oil.

### 6. Synthesis of 288f

To a stirred mixture of 288e (1.2 g, 3.657 mmol, 1 equiv) in tetrahydrofuran (13 mL) was added methyl isothiocyanate (0.59 g, 8.045 mmol, 2.2 equiv) at room temperature. The resulting mixture was stirred overnight at room temperature. The resulting mixture was concentrated under vacuum. The precipitated solids were collected by filtration and washed with water (2×10 mL). This resulted in 288f (1.1 g, 67.47%) as a white solid.

### 7. Synthesis of 288g

To a stirred mixture of NaOH (0.44 g, 10.964 mmol, 4 equiv) in H2 O (12 mL) was added 288f (1.1 g, 2.741 mmol, 1 equiv) at room temperature. The resulting mixture was stirred overnight at room temperature,The mixture was acidified to pH 5 with 1M HCl (aq.).The aqueous layer was extracted with EtOAc (2×30 mL). The combined organic layers were concentrated under vacuum. This resulted in 288g (1 g, 86.62%) as a yellow oil. The crude product was used in the next step directly without further purification.

### 8. Synthesis of 288h

To a stirred mixture of 288g (1 g, 2,609 mmol, 1 equiv) and NaNO2 (1.80 g, 26.090 mmol, 10 equiv) in H2 O (20 mL) was added HNO3 (26.09 mL, 26.090 mmol, 10 equiv, 1M) at 0° C. The resulting mixture was stirred overnight at room temperature. The mixture was basified to pH 7 with saturated NaHCO₃ (aq.). The aqueous layer was extracted with EtOAc (2×30 mL).The resulting mixture was concentrated under vacuum. The residue was purified by Prep-TLC (DCM/MeOH 20:1) to afford 288h (900 mg, 90.36%) as a yellow oil.

### 9. Synthesis of 288i

To a solution of 288h (780 mg, 2.221 mmol, 1 equiv) and Zn(CN)2 (1043.13 mg, 8.884 mmol, 4 equiv) in NMP (10 mL) was added Pd(PPh3)4 (256.65 mg, 0.222 mmol, 0.1 equiv) at room temperature under nitrogen atmosphere. The final reaction mixture was irradiated with microwave radiation for 3h at 140° C. The reaction was quenched with water (100 mL) at room temperature. The aqueous layer was extracted with EtOAc (2×50 mL). The organic layers were concentrated under reduced pressure. The residue was purified by Prep-TLC (DCM/MeOH 40:1) to afford 288i (360 mg, 49.07%) as a light yellow solid,

### 10, Synthesis of 288j

To a stirred mixture of 288i (350 mg, 1.177 mmol, 1 equiv) and NH4Cl (629.68 mg, 11.770 mml, 10 equiv) in EtOH (5 mL) was added H2 O (3 mL) and Fe (197.22 mg, 3.531 mmol, 3 equiv) at room temperature. The resulting mixture was stirred for 6h at 80° C. The resulting mixture was filtered through a Celite pad, the filter cake was washed with EtOAc (2×5 mL). The filtrate was concentrated under reduced pressure. The residue was purified by Prep-TLC (DCM/MeOH 20:1) to afford 288j (210 mg, 63.39%) as a white solid.

### 11. Synthesis of 288k

To a stirred solution of 288j (200 mg, 0.748 mmol, 1 equiv) and I-2 (642.56 mg, 2.244 mmol, 3 equiv) in DCE (5 m L) was added NaBH(OAc)3 (317.11 mg, 1.496 mmol, 2 equiv) at room temperature. The resulting mixture was stirred overnight at room temperature. The reaction was quenched with saturated NaHCO₃ (aq.) (50 mL) at room temperature. The aqueous layer was extracted with DCM (2×30 mL). The organic layers were concentrated under reduced pressure. The residue was purified by Prep-TLC (DCM/MeOH 20:1) to afford 288k (200 mg, 46.24%) as an oft-white solid.

### 12. Synthesis of 2881

To a stirred solution of 288k (180 mg, 0.335 mmol, 1 equiv) and Pyridine (264.83 mg, 3.350 mmol, 10 equiv) in DCM (5 mL) was added Triphosgene (49.67 mg, 0.168 mmol, 0.5 equiv) at room temperature. The resulting mixture was stirred for 10 min at room temperature. The reaction was quenched with saturated NaHCO₃ (aq.) (50 mL) at room temperature. The aqueous layer was extracted with DCM (2×20 mL). The organic layers were concentrated under reduced pressure. The residue was purified by Prep-TLC (DCM/MeOH 18:1) to afford 2881 (120 mg, 61.05%) as a yellow solid,

### 13. Synthesis of 288

The 2881 (120 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:11; Flow rate: 20 L/min; Gradient: 50% B to 50% B in 14 min; Wave Length: 220/254 nm; RT1 (min): 9.33; RT2 (min): 12.09; the first peak is product) to afford 288 (32.6 mg, 26.95%) as a yellow solid.

LC-MS: (ES, m/z): [M+H]+ 564. H-NMR: (400 MHz, DMSO-d6, δ ppm): 0.79-0.93 (m, 4H), 1.43-1.53 (m, 1H), 1.58-1.69 (m, 4H), 1.71-1.93 (m, 6H), 2.03-2.08 (m, 1H), 2.64-2.83 (m, 2H), 3.19-3.30 (m, 3H), 3.48 (s, 3H), 4.38-4.40 (d, 1H), 7.03 (s, 1H), 7.52 (s, 1H), 7.65-7.70 (m, 2H), 8.23-8.24 (m, 2H), 8.37 (s, 1H).

### Example 289. Synthesis of Compound 289

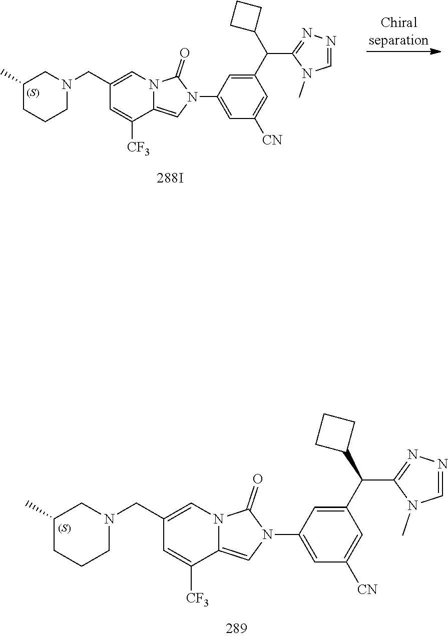

### Synthesis of 289

288l (120 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 50% B to 50% B in 14 min; Wave Length: 220/254 nm; RT1 (min): 9.33; RT2 (min): 12.09; the second peak is product) to afford 289 (37.8 mg, 31.18%) as a yellow solid,

LC-MS: (ES, m/z): [M+H]+ 564. H-NMR: (400 MHz, DMSO-d6, δ ppm): 0.79-0.93 (m, 4H), 1.41-1.98 (m, 1H), 2.03-2.08 (m, 1H), 2.64-2.83 (m, 2H), 3.19-3.30 (m, 3H), 3.48 (s, 3H), 4.38-4.40 (d, 1H), 7.03 (s, 1H), 7.52 (s, 1H), 7.66-7.70 (m, 2H), 8.23-8.24 (m, 2H), 8.37 (s, 1H).

### Example 290. Synthesis of Compound 290

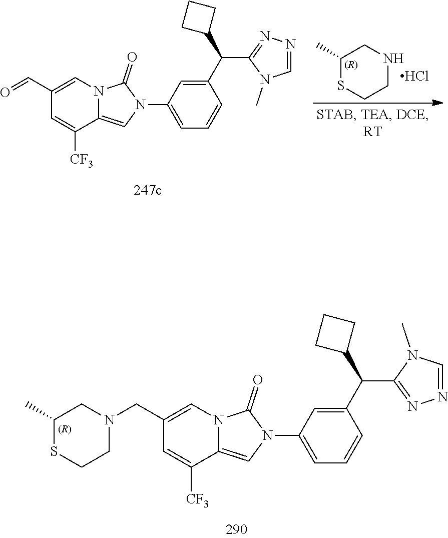

### Synthesis of 290

To a stirred solution of 247c (200 mg, 0.439 mmol 1 equiv) and (2R)-2-methylthiomorpholine hydrochloride (134.96 mg, 0.878 mmol, 2 equiv) in DCE (5 mL) was added TEA (88.87 mg, 0.878 mmol, 2 equiv) at room temperature. The resulting mixture was stirred for 1h at room temperature. To the above mixture was added STAB (186.14 mg, 0.878 mmol, 2 equiv) at room temperature. The resulting mixture was stirred for additional overnight at room temperature. The reaction was quenched with saturated NH4Cl (aq.) (50 mL) at room temperature. The aqueous layer was extracted with DCM (2×30 mL). The organic layers were concentrated under reduced pressure. The residue was purified by Prep-TLC (DCM/MeOH 20:1) to afford the crude product. The residue was purified by reverse flash chromatography with the following conditions: column, C18 silica gel; mobile phase, MeCN in water, 0% to 50% gradient in 20 min; detector, UV 254 nm. This resulted in 290 (64.0 mg, 25.29%) as a yellow solid.

LC-MS: (ES, m/z): [M+H]+ 557. H-NMR: (400 MHz, DMSO-d6, δ ppm): 1.18-1.24 (d, 3H), 1.69-1.93 (m, 5H), 2.01-2.15 (m, 2H), 2.24-2.33 (m, 1H), 2.51-2.59 (m, 1H), 2.71-2.77 (m, 1H), 2.87-2.91 (m, 1H), 2.98-3.01 (m, 2H), 3.21-3.25 (m, 1H), 3.32 (s, 2H), 3.43 (s, 3H), 4.25-4.28 (d, 1H), 7.01 (s, 1H), 7.19-7.21 (d, 1H), 7.33 (s, 1H), 7.43-7.47 (m, 1H), 7.69-7.71 (m, 2H), 7.74 (s, 1H), 8.34 (s, 1H).

### Example 291. Synthesis of Compound 291

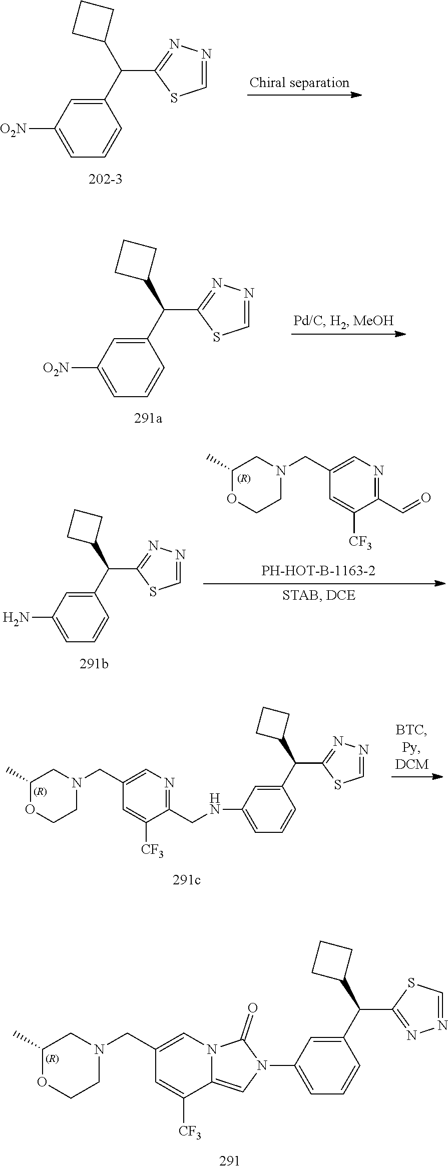

### 1. Synthesis of 291a

202-3 (1 g, 3,559 mmol, 1 equiv) was purified by Prep-SFC with the following conditions (Column: (R, R) WHELK-O1-Kromasil, 5*25 cm, 5 μm; Mobile Phase A: CO2, Mobile Phase B: MeOH; Flow rate: 200 n/min; Gradient: isocratic 35% B; Column Temperature (° C.): 35; Back Pressure (bar): 100; Wave Length: 220 am; RT1 (min): 5.08; RT2 (min): 6.08; the first peak was product) to afford 291 a (0.397 g, 40.51%) as a yellow oil.

### 2. Synthesis of 292b

Into a 50 mL round-bottom flask were added 291a (397.1 mg, 1.442 mmol, 1 equiv) and Pd/C (122.79 mg 10%) in MeOH (4 mL) at room temperature. The resulting mixture was stirred for overnight at room temperature under hydrogen atmosphere. The resulting mixture was filtered, the filter cake was washed with MeOH (3×10 mL). The filtrate was concentrated under reduced pressure. This resulted in 292b as light yellow solid.

### 3. Synthesis of 292c

A solution of 292b (200 mg, 0.815 mmol, 1 equiv) in DCE (4 mL) was treated with 330-2 (258.50 mg, 0.896 mmol, 1.1 equiv) for 2h at room temperature under nitrogen atmosphere followed by the addition of STAB (259.16 mg, 1.222 mmol, 1.5 equiv) at room temperature. The resulting mixture was stirred for overnight at room temperature. The reaction was quenched with sat. NaHCO3 (aq.)(8 ml) at room temperature. The resulting mixture was extracted with DCE/MeOH (10:1) (3×20 mL). The combined organic layers were concentrated under reduced pressure. The residue was purified by reverse flash chromatography with the following conditions: column, C18 silica gel; mobile phase A: Water (0.1% NH4HCO3); mobile phase B: ACN,Gradient: 20% B to 80% B in 30 min; detector, UV 254 nm. This resulted in 292c (300 mg, 71.10%) as a yellow solid.

### 4. Synthesis of 292

A solution of 292c (310 mg, 0.599 mmol, 1 equiv) in DCM (9 mL) was treated with Pyridine (284.24 mg, 3.594 mmol, 6 equiv) at room temperature followed by the addition of triphosgene (71.08 mg, 0.240 mmol, 0.4 equiv) at 0° C. The resulting mixture was stirred for 30 min at room temperature. The reaction was quenched by the addition of sat. NaHCO₃ (8 mL) at room temperature. The resulting mixture was extracted with DCM/MeOH (10:1)(2×10 mL). The combined organic layers were concentrated under reduced pressure. The residue was purified by reverse flash chromatography with the following conditions: column, Xselect CSH C18 OBD; mobile phase, A: Water (0.1% NH4HCO3), Mobile Phase B: ACN, 20% B to 75% B in 30 min; UV 254 nm. This resulted in 292 (246 mg, 75.56%) as a yellow solid,

LC-MS: (ES, m/z): [M+H]+ 544. H-NMR: (400 MHz, DMSO, ppm): δ 1.03-1.05 (d, 3H), δ 1.65-1.85 (m, 6H), δ 2.00-2.11 (m, 2H), δ 2.63-2.75 (m, 2H), δ 3.20-3.29 (m, 3H), δ 3.34-3.40 (m, 2H), δ 3.71-3.77 (m, 1H), δ 4.71-4.74 (d, 1H), δ 7.02 (s, 1H), δ 7.35-7.38 (m, 2H), δ 7.46-7.48 (m, 1H), 7.70 (s, 1H), δ 7.74-7.82 (m, 1H), δ 7.88 (s, 1H), δ 9.50 (s, 1H).

### Example 292. Synthesis of Compound 292

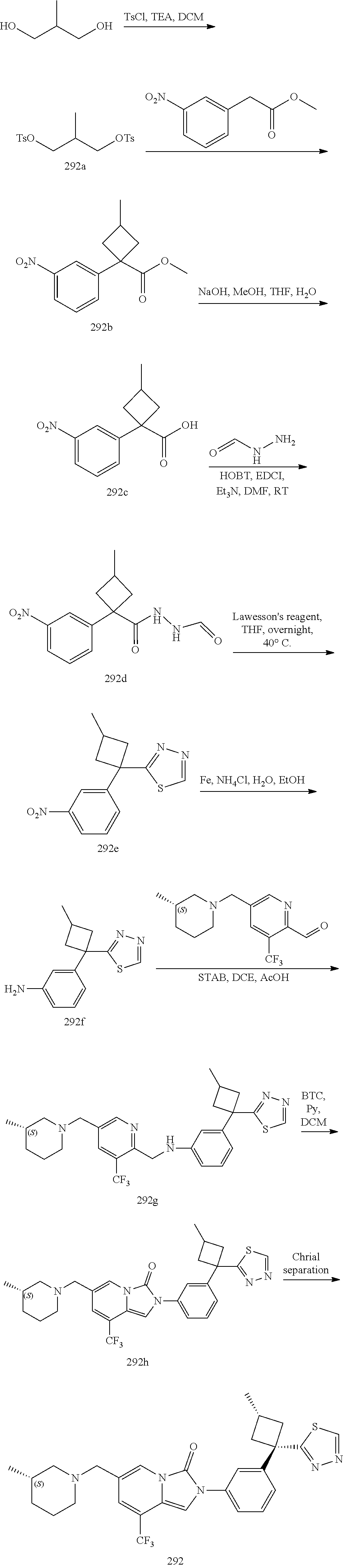

### 1. Synthesis of 292a

Into a 1 L 3-necked round-bottom flask were added methylpropanediol (50 g, 554.803 mmol, 1 equiv) and TsCl (317.30 g, 1664.409 mmol, 3 equiv) in DCM (500 mL) at room temperature. To the above mixture was added TEA (280.71 g, 2774.015 mmol, 5 equiv) dropwise over 1h at room temperature. The resulting mixture was stirred for additional overnight at room temperature. The resulting mixture was diluted with water (2 L). The aqueous layer was extracted with CH2Cl2 (3×500 mL). The combined organic layers were concentrated under reduced pressure. The residue was purified by silica gel column chromatography, eluted with CH2Cl2/MeOH (100:1) to afford 292a (110 g, 49.75%) as a white solid. 2. Synthesis of 292b

To a stirred mixture of methyl methyl 2-(3-nitrophenyl)acetate (35 g, 179,327 mmol, 1.00 equiv) in DMF (350 mL) was added Cs2CO3 (292.14 g, 896,635 mmol, 5 equiv) at 0° C. under nitrogen atmosphere. The resulting mixture was stirred for 2h at 0° C. under nitrogen atmosphere. To the above mixture was added 292a (107.19 g, 268.990 mmol, 1.5 equiv) at 0° C. The resulting mixture was stirred for additional 24h at room temperature. The reaction was quenched by the addition of sat. NH4Cl (aq.) (2 L) at room temperature. The aqueous layer was extracted with EtOAc (2×1 L). The combined organic layers were dried over anhydrous Na2SO4. After filtration, the filtrate was concentrated under reduced pressure. The residue was purified by silica gel column chromatography, eluted with PE/EA (5:1) to afford methyl 202b (10 g, 22.37%) as a yellow oil.

### 3. Synthesis of 292c

To a stirred mixture of 292b (4.7g, 18.855 mmol, 1 equiv) in MeOH (10 ml) and THF (10 ml) was added NaOH (2.26 g, 56.565 mmol, 3 equiv) in H2 O (10 mL) at room temperature. The mixture was stirred for 2h at room temperature. The mixture was acidified to pH 5 with 1M HCl (aq.). The resulting mixture was extracted with EA (3×30 ml). The combined organic layers were dried over anhydrous Na2SO4. After filtration, the filtrate was concentrated under reduced pressure. This resulted in 292c (4 g, 90.18%) as a white solid.

### 4. Synthesis of 292d

To a stirred of 292c (3.98 g, 16.919 mmol, 1 equiv) and and N-formylhydrazine (1.02 g, 16.919 mmol, 1 equiv) in DMF (40 mL) were added HOBT (3.43 g, 25.379 mmol, 1.5 equiv) and EDCI (4.87 g, 25,379 mmol, 1.5 equiv) at room temperature. To the above mixture was added TEA (2.57 g, 25.379 mmol, 1.5 equiv) dropwise over 30 min at room temperature. The resulting mixture was stirred for additional overnight at room temperature. The reaction was quenched by the addition of sat. NH4Cl (aq.) (150 mL) at room temperature. The aqueous layer was extracted with EtOAc (3×50 mL).The combined organic layers were concentrated under reduced pressure. The residue was purified by silica gel column chromatography, eluted with CH2Cl2/MeOH (100:1) to afford 292d (2.8 g, 59.69%) as a yellow oil,

### 5. Synthesis of 292e

Into a 100 ml 3-necked round-bottom was added 292d (2.775 g, 10.008 mmol, 1 equiv), Lawesson's Reagent (8.10 g, 20.016 mmol, 2 equiv), and THF (28 mL) at room temperature. The mixture was stirred overnight at 40° C. The residue was diluted with water (90 ml). The aqueous layer was extracted with EA (3×30 ml). The resulting mixture was concentrated under reduced pressure. The residue was purified by silica gel column chromatography, eluted with DCM: MeOH (100:1) to afford 292e (1.2 g, 43.55%) as a yellow oil.

### 6. Synthesis of 292f

Into a 25 ml flasket were added 292e (1.2 g, 4,358 mmol, 1 equiv) and Fe (0.49 g, 8.716 mmol, 2 equiv), NH4Cl (0.699 g, 13.074 mmol, 3 equiv) and EtOH (6 mL) at room temperature. The resulting mixture was stirred overnight at 80° C. The mixture was allowed to cool down to room temperature. The resulting mixture was filtered; the filters cake was washed with EA (2×20 mL). The filtrate was concentrated under reduced pressure. The resulting mixture was diluted with water (20 mL). The aqueous layer was extracted with EtOAc (3×20 mL). The combined organic layers concentrated under reduced pressure. This resulted in 292f (1 g, 93.52%) as a yellow oil.

### 7. Synthesis of 292g

Into a 25 mL round-bottom flask were added 292f (500 mg, 2.038 mmol, 1 equiv) and I-2 (641.82 mg, 2.242 mmol, 1.1 equiv) in DCE (5 mL) at room temperature. To the above mixture was added AcOH (122.39 mg, 2.038 mmol, 1 equiv) and STAB (863.86 mg, 4.076 mmol, 2 equiv) at room temperature. The resulting mixture was stirred for additional 8h at room temperature. The resulting mixture was diluted with water (25 mL). The aqueous layer was extracted with CH2Cl2 (3×20 ml). The resulting mixture was concentrated under reduced pressure. The residue was purified by Prep-TLC (DCM: MeOH 20:1) to afford 292g (800 mg, 76.13%) as a yellow oil.

### 8. Synthesis of 292h

Into a 25 mL round-bottom flask were added Pyridine (460.20 mg, 5.820 mmol, 6 equiv) and 292g (500 mg, 0.970 mmol, 1 equiv) in DCM (5 mL) at room temperature. To the above mixture was added BTC (115.10 mg, 0.388 mmol, 0.4 equiv) in three portions at room temperature. The resulting mixture was stirred for 2h at room temperature under N2 atmosphere. The reaction was quenched by the addition of sat. NaHCO₃ (aq.) (25 mL) at room temperature. The resulting mixture was extracted with DCM (3×20 ml). The combined organic layers were concentrated under reduced pressure. The residue was purified by Prep-TLC (DCM: MeOH 25:1) to afford 292h as a yellow solid,

### 9. Synthesis of 292

The 292h was purified by Prep-Chiral-HPLC with the following conditions (Column: CHIRALPAK IH, 2*25 cm, 5 μm; Mobile Phase A: Hex (0.5% 2M NH3-MeOH), Mobile Phase B: EtOH:DCM=1:1; Flow rate: 20 mL/min; Gradient: 10% B to 10% B in 10.5 min; Wave Length: 220/254 nm; RT1 (min): 6.26; RT2 (min): 8.10; Sample Solvent: EtOH: DCM=1:1; the first peak was product) to afford 292 (167.1 mg, 54.64%) as a yellow solid.

H-NMR: (400 MHz, dmso-d6, δ ppm): 0.81-0.89 (m, 4H), 1.08-1.13 (d, 3H), 1.40-1.49 (m, 1H), 1.58-1.66 (m, 4H), 1.87-1.92 (m, 1H), 2.32-2.38 (m, 1H), 2.58-2.67 (m, 2H), 2.73-2.77 (m, 2H), 3.06-3.11 (m, 2H), 3.25-3.34 (m, 2H), 7.00 (s, 1H), 7.45-7.48 (m, 2H), 7.51-7.56 (m, 1H), 7.66 (s, 1H), 7.77-7.79 (m, 1H), 8.00 (s, 1H), 9.51 (s, 1H).

### Example 293. Synthesis of Compound 293

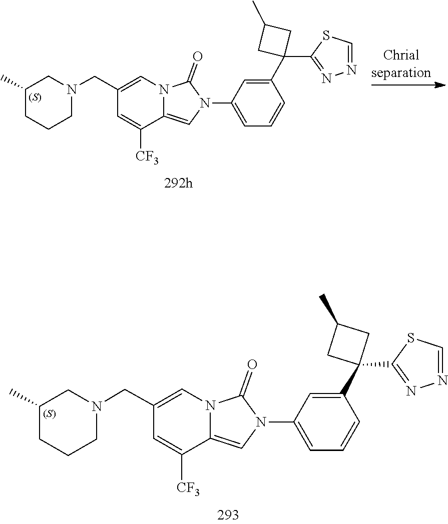

### Synthesis of 293

292h was purified by Prep-Chiral-HPLC with the following conditions (Column: CHIRALPAK IH, 2*25 cm, 5 μm; Mobile Phase A: Hex (0.5% 2M NH3-MeOH), Mobile Phase B: EtOH:DCM=1:1; Flow rate: 20 mL/min; Gradient: 10% B to 10% B in 10.5 min; Wave Length: 220/254 nm; RT1 (min): 6.26; R T2 (min): 8.10; the second peak was product) to afford 293 (42.2 mg, 13.41%) as a yellow solid.

LC-MS: (ES, m/z): [M+H+ 542. H-NMR: (400 MHz, DMSO-d6, δ ppm): 0.81-0.89 (m, 4H), 1.08-1.13 (d, 3H), 1.40-1.49 (m, 1H), 1.58-1.66 (m, 4H), 1.87-1.92 (m, 1H), 2.40-2.45 (m, 2H), 2.58-2.64 (m, 1H), 2.73-2.80 (m, 2H), 3.06-3.15 (m, 2H), 3.25-3.34 (m, 2H), 7.00 (s, 1H), 7.45-7.48 (m, 2H), 7.51-7.56 (m, 1H), 7.66 (s, 1H), 7.77-7.79 (m, 1H), 8.00 (s, 1H), 9.51 (s, 1H).

### Example 294. Synthesis of Compound 294

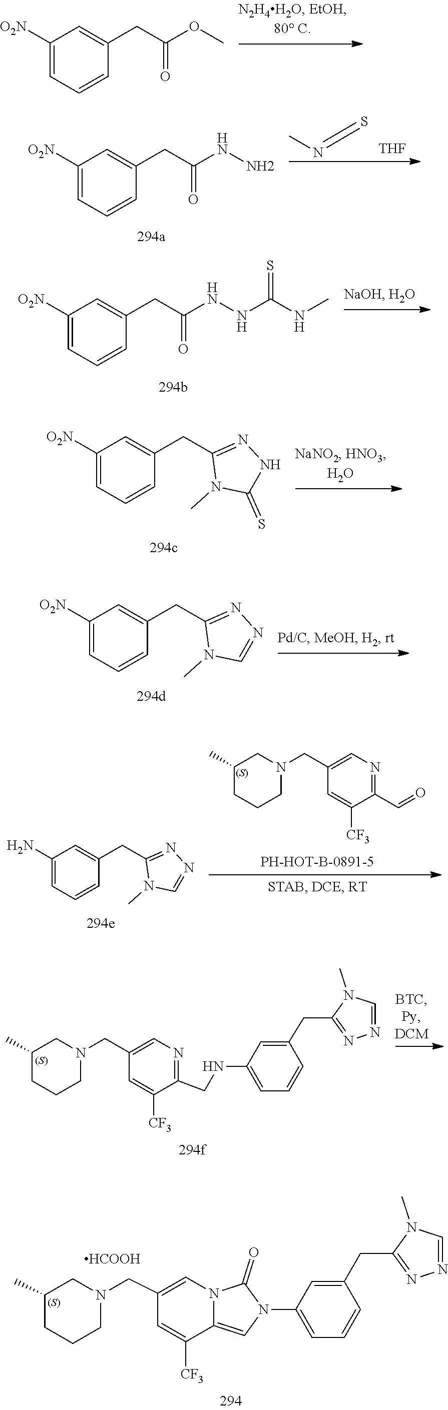

### 1. Synthesis of 294a

A mixture of methyl 2-(3-nitrophenyl)acetate (10 g, 51.236 mmol, 1.00 equiv) and hydrazine hydrate (98%) (25.65 g, 512.360 mmol, 10 equiv) in EtOH (50 mL) was stirred for overnight at 80° C. The reaction was quenched with water (200 mL) at room temperature. The aqueous layer was extracted with CH2Cl2:MeOH=10:1 (4×300 mL). The resulting mixture was concentrated under reduced pressure. This resulted in 294a (8.2 g, 82.00%) as a white solid.

### 2. Synthesis of 294b

Into a 100 mL 3-necked round-bottom flask were added methyl isothiocyanate (7.49 g, 102.470 mmol, 2.5 equiv) and 294a (8 g 40.988 mmol, 1 equiv) in tetrahydrofuran (80 mL) at room temperature. The resulting mixture was stirred for overnight at room temperature. The resulting mixture was dilated with water (80 mL). The THF was removed under reduced pressure. The precipitated solids were collected by filtration and washed with water (20 mL). This resulted in 294b (11 g, 85.03%) as a white solid.

### 3. Synthesis of 294c

Into a 500 mL 3-necked round-bottom flask were added NaOH (150 mL, 1M) and 294b (11 g, 41.000 mmol, 1 equiv) at room temperature. The resulting mixture was stirred for overnight at room temperature. The mixture was acidified to pH 6 with HCl (aq.) (1 M). The aqueous layer was extracted with CH2Cl2:MeOH=10:1 (3×200 mL).The resulting mixture was concentrated under reduced pressure to afford 294c (7.5 g, 73.09%) as a white solid.

### 4. Synthesis of 294d

Into a 1 L 3-necked round-bottom flask were added NaNO2 (21 g, 304.370 mmol) in H2O (50 mL), EtOAc (50 mL) and 294c (7.5 g, 9.989 mmol, 1 equiv) at room temperature. To the above mixture was added HNO3 (300 mL, 10 equiv, 1M in H2O) at 0° C. The resulting mixture was stirred for additional overnight at room temperature. The reaction was quenched by the addition of NaHCO3 (aq.) (200 mL) at room temperature. The aqueous layer was extracted with EtOAc (3×500 mL). The resulting mixture was concentrated under reduced pressure. The residue was purified by silica gel column chromatography, eluted with PE/EA=10:1 to afford 294d (5.6 g, 231.22%) as a yellow solid.

### 5. Synthesis of 294e

To a stirred solution of 294d (5.6 g, 25.663 mmol, 1 equiv) in MeOH (110 mL) was added Pd/C (1.2 g) at room temperature under nitrogen atmosphere. The resulting mixture was stirred for overnight at room temperature under hydrogen atmosphere using a hydrogen balloon, filtered through a Celite pad and concentrated under reduced pressure. This resulted in 294e (4.9 g, 90.28%) as a yellow solid.

### 6. Synthesis of 294f

To a stirred mixture of 294e (200 mg, 1,063 mmol, 1 equiv) and 1-2 (304.19 mg, 1.063 mmol, 1 equiv) in DCE (3 mL) was added STAB (675.56 mg, 3.189 mmol, 3 equiv) at room temperature. The resulting mixture was stirred for overnight at room temperature. The reaction was quenched by the addition of NaHCO₃ (aq.) (30 mL) at room temperature. The aqueous layer was extracted with CH2Cl2 (2×30 mL). The resulting mixture was concentrated under vacuum. The residue was purified by Prep-TLC (CH2Cl2/MeOH=15:1) to afford 294f (220 mg, 41.09%) as a yellow solid.

### 7. Synthesis of 294

To a stirred solution of 294f (300 mg, 0,654 mmol, 1 equiv) and Pyridine (310.51 mg, 3,924 mmol, 6 equiv) in DCM (6 mL) was added Triphosgene (97.07 mg, 0.327 mmol, 0.5 equiv) at 0° C. The resulting mixture was stirred for 30 min at 0° C. The reaction was quenched by the addition of NaHCO₃ (aq.) (30 mL) at room temperature. The aqueous layer was extracted with CH2Cl2 (2×20 mL). The resulting mixture was concentrated under reduced pressure. The residue was purified by Prep-TLC (CH2Cl2/MeOH=15:1) to afford the crude product (150 mg). The crude product was purified by reverse phase flash with the following conditions (Mobile Phase A: H2O (0.1% HCOOH), Mobile Phase B: MeCN; Flow rate: 20 mL/min; Gradient: 2% B to 100% B in 30 min; Wave Length: 220/254 nm) to afford 294 (76.6 mg, 21.85%) as a yellow solid.

LCMS: (ES, m/z): [M+H]+ 485. H-NMR: (400 MHz, DMSO-d6, ppm, δ): 0.90-0.98 (m, 4H), 1.43-1.49 (m, 1H), 1.59-1.66 (m, 4H), 1.88-1.93 (m, 1H), 2.67-2.78 (m, 2H), 3.26 (s, 2H), 3.53 (s, 3H), 4.24 (s, 2H), 7.01 (s, 1H), 7.17-7.19 (d, 1H), 7.32 (s, 1H), 7.45-7.48 (t, 1H), 7.67-7.74 (m, 3H), 8.15 (s, 1H), 8.39 (s, 1H).

### Example 295. Synthesis of Compound 295

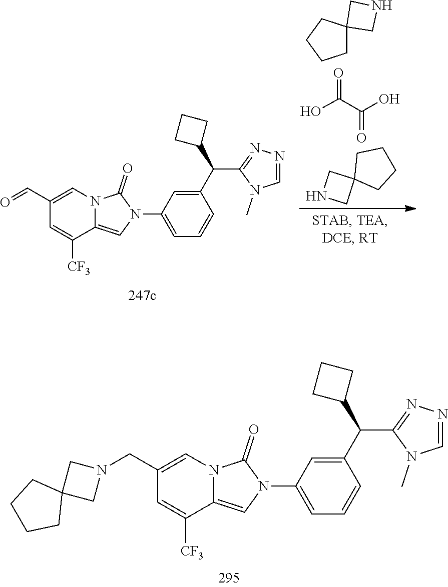

### Synthesis of 295

To a stirred solution of 247c (200 mg, 0.439 mmol, 1 equiv) and 2-Azaspiro[3,4]octane hemioxalate (2:1) (137.19 mg, 0,439 mmol, 1 equiv) in DCE (5 mL) was added TEA (88.87 mg, 0.878 mmol, 2 equiv) at room temperature. The resulting mixture was stirred for 1h at room temperature. To the above mixture was added STAB (186.14 mg, 0,878 mmol, 2 equiv) at room temperature. The resulting mixture was stirred overnight at room temperature. The reaction was quenched with saturated NH4Cl (aq.) (50 ml) at room temperature. The aqueous layer was extracted with DCM (2×30 mL). The organic layer: were concentrated under reduced pressure. The residue was purified by Prep-TLC (DCM/MeOH 20:1) to afford the crude product. The crude product was purified by Prep-HPLC with the following conditions (Column: XBridge Prep OBD C18 Column, 30*150 mm, 5 μm; Mobile Phase A: Water (10 mmol/L NH41HCO3), Mobile Phase B: ACN; Flow rate: 60 mL/min; Gradient: 39% B to 69% B in 8 min, Wave Length: 220 nm; RT1 (min): 7.23) to afford 295 (45.5 mg, 18.72%) as a yellow solid.

LC-MS: (ES, m/z): [M+H]+ 551. H-NMR0: (400 MHz, DMSO-d6, δ ppm): 1.48-1.51 (m, 4H), 1.69-1.74 (m, 5H), 1.74-1.85 (m, 4H), 2.07-2.10 (m, 1H), 3.05 (s, 4H), 3.18-3.25 (m, 1H), 3.32 (s, 2H), 3.43 (s, 3H), 4.24-4.27 (d, 1H), 6.97 (s, 1H), 7.18-7.20 (d, 1H), 7.31 (s, 1H), 7.42-7.46 (m, 1H), 7.63 (s, 1H), 7.68-7.70 (m, 1H), 7.74 (s, 1H), 8.33 (s, 1H).

### Example 296. Synthesis of Compound 296

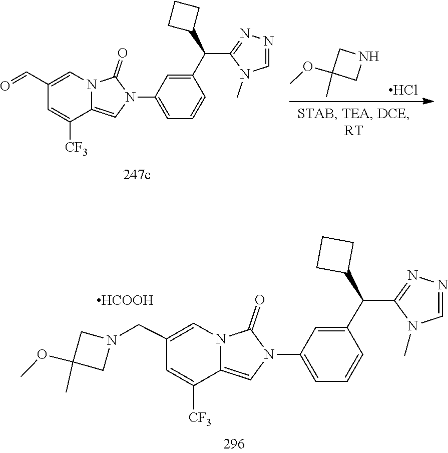

### Synthesis of 296

To a stirred solution of 247c (300 mg, 0.659 mmol, 1 equiv) and 3-methoxy-3-methylazetidine hydrochloride (181.29 mg, 1.318 mmol, 2 equiv) in DCE (5 mL) was added TEA (133.31 mg, 1318 mmol, 2 equiv) at room temperature. The resulting mixture was stirred for 1h at room temperature. To the above mixture was added STAB (279.21 mg, 1,318 mmol, 2 equiv) at room temperature. The resulting mixture was stirred overnight at room temperature. The reaction was quenched with saturated NH4Cl (aq.) (50 ml) at room temperature. The aqueous layer was extracted with DCM (2×30 mL). The organic layers were concentrated under reduced pressure. The residue was purified by Prep-TLC (DCM/MeOH 20:1) to afford the crude product. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 16% B to 30% B in 7 ruin, Wave Length: 254; 220 nm; RT1 (min): 5.73) to afford 296 (84.4 mg, 21.54%) as a yellow solid.

LC-MS: (ES, m/z): [M+H]+ 541. H-NMR: (400 MHz, DMSO-d6, δ ppm): 1.41 (s, 3H), 1.63-1.93 (m, 5H), 2.07-2.12 (m, 1H), 3.04-3.06 (m, 2H), 3.10 (s, 3H), 3.14-3.18 (m, 2H), 3.24-3.28 (m, 2H), 3.43 (s, 4H), 4.25-4.27 (d, 1H), 6.99 (s, 1H), 7.18-7.20 (d, 1H), 7.32 (s, 1H), 7.42-7.46 (m, 1H), 7.68-7.70 (m, 2H), 7.74 (s, 1H), 8.15 (s, 1H), 8.33 (s, 1H).

### Example 297. Synthesis of Compound 297

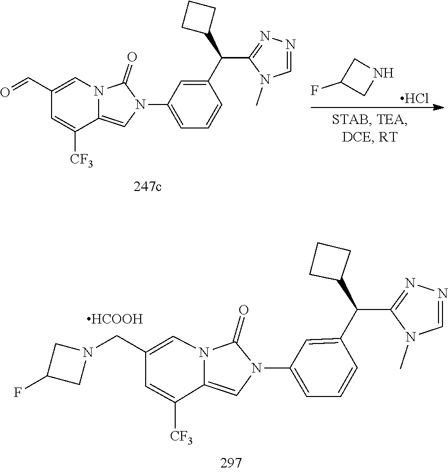

### Synthesis of 297

To a stirred solution of 247c (300 mg 0.659 mmol, 1 equiv) and 3-fluoroazetidine hydrochloride (146.94 mg, 1.318 mmol, 2 equiv) in DCE (5 mL) were added TEA (133.31 mg, 1318 mmol, 2 equiv) at room temperature. The resulting mixture was stirred for 1h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×30 mL). The organic layers were concentrated under reduced pressure. The residue was purified by Prep-TLC (DCM/MeOH 20:1) to afford the crude product. The crude product was purified by Prep-HPLC with the following conditions (Column: Xselect CSH C18 OBD Column 30*150 mm 5n, n; Mobile Phase A: Water (0.1% FA), Mobile Phase B: ACN; Flow rate: 60 mL/min; Gradient: 11% B to 30% B in 7 min, Wave Length: 254; 220 am; RT1 (min): 6.40) to afford 297 (99.5 mg, 26.60%) as a yellow solid,

LC-MS: (ES, m/z): [M+H]+ 515. H-NMR: (400 MHz, DMSO-d6, δ ppm): 1.63-1.93 (m, 5H), 2.07-2.12 (m, 1H), 3.15-3.18 (m, 3H), 3.24-3.27 (m, 1H), 3.35-3.39 (m, 2H), 3.43 (s, 2H), 3.54-3.65 (m, 2H), 4.25-4.27 (d, 1H), 5.11-5.29 (m, 1H), 6.98 (s, 1H), 7.18-7.20 (d, 1H), 7.31 (s, 1H), 7.42-7.46 (m, 1H), 7.68-7.73 (m, 3H), 8.14 (s, 1H), 8.33 (s, 1H).

### Example 298. Synthesis of Compound 298

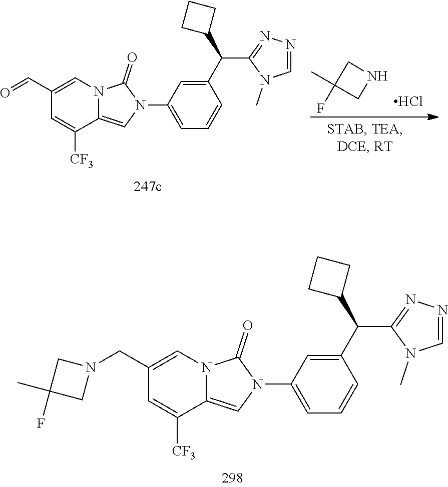

### Synthesis of 298

To a stirred solution of 247c (300 mg, 0.659 mmol, 1 equiv) and 3-fluoro-3-methylazetidine hydrochloride (165.43 mg, 1,318 mmol, 2 equiv) in DCE (5 mL) were added TEA (133.31 mg, 1.318 mmol, 2 equiv) at room temperature. The resulting mixture was stirred for 1 b at room temperature. To the above mixture was added STAB (279.21 mg, 1.318 mmol, 2 equiv) at room temperature. The resulting mixture was stirred overnight at room temperature. The reaction was quenched with saturated NH₄Cl (aq.) (50 mL) at room temperature. The aqueous layer was extracted with DCM (2×30 mL). The organic layers were concentrated under reduced pressure. The residue was purified by Prep-TLC (DCM/MeOH 20:1) to afford the crude product. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 60% B in 8 min, Wave Length: 220 am; RT1 (min): 7.32) to afford 298 (95.1 mg, 27.21%) as a yellow solid.

LC-MS: (ES, m/z): [M+H]+ 529. H-NMR: (400 MHz, DMSO-d6, δ ppm): 1.50-1.55 (m, 3H), 1.63-1.88 (m, 5H), 2.07-2.12 (m, 1H), 3.20-3.31 (m, 5H), 3.43-3.44 (m, 5H), 3.54-3.65 (m, 2H), 4.25-4.27 (d, 1H), 6.98 (s, 1H), 7.18-7.20 (d, 1H), 7.32 (s, 1H), 7.42-7.46 (m, 1H), 7.68-7.72 (m, 2H), 7.72-7.74 (m, 1H), 8.33 (s, 1H).

### Example 299. Synthesis of Compound 299

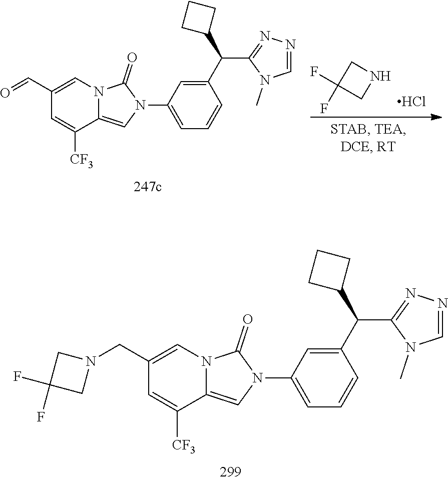

### Synthesis of 299

To a stirred solution of 247c (300 mg, 0.659 mmol, 1 equiv) and 3,3-difluoroazetidine hydrochloride (170.64 mg, 1.318 mmol, 2 equiv) in DCE (5 mL) were added TEA (133.31 mg, 1.318 mmol, 2 equiv) at room temperature. The resulting mixture was stirred for 1b at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×30 mL). The organic layers were concentrated under reduced pressure. The residue was purified by Prep-TLC (DCM/MeOH 20:1) to afford the crude product. The crude product was purified by Prep-HPLC with the following conditions (Column: XBridge Shield RP18 OBD Column, 30*150 mm, 5 um; Mobile Phase A: Water (10 mmol/L NH4HCO3+0.1% NH3,H2O), Mobile Phase B: ACN; Flow rate: 60 mL/min; Gradient: 34% B to 54% B in 8 min, 54% B; Wave Length: 220 nm; RT1 (min): 7.70) to afford 299 (50.7 mg, 13.82%) as a yellow solid.

LC-MS: (ES, m/z): [M+H]+ 533. H-NMR: (400 MHz, DMSO-d6, δ ppm): 1.63-1.88 (m, 5H), 2.07-2.12 (m, 1H), 3.20-3.23 (m, 1H), 3.41 (s, 3H), 3.53 (s, 2H), 3.66-3.66 (m, 4H), 4.25-4.27 (d, 1H), 7.00 (s, 1H), 7.19-7.21 (d, 1H), 7.32 (s, 1H), 7.42-7.46 (m, 1H), 7.68-7.76 (m, 3H), 8.33 (s, 1H).

### Example 300. Synthesis of Compound 300

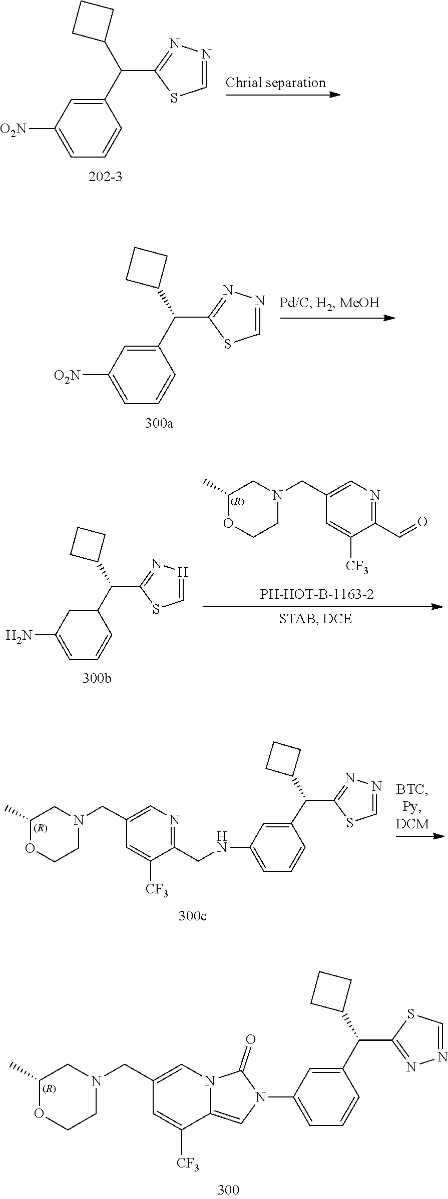

### 1. Synthesis of 300a

202-3 (1 g, 3.559 mmol, 1 equiv) was purified by Prep-SFC with the following conditions (Column: (R, R)-WHELK-O1-Kromasil, 5*25 cm, 5 μm; Mobile Phase A: CO2, Mobile Phase B: MeOH; Flow rate: 200 mL/min; Gradient: isocratic 35% B; Column Temperature (° C.): 35; Back Pressure (bar): 100; Wave Length: 220 nm; RT1 (min): 5.08; RT2 (min): 6.08; second peak was product)) to afford 300a (0.469 g, 46.90%) as a yellow oil, 2. Synthesis of 300b

Into a 50 mL round-bottom flask were added 300a (0.469 g, 46.90%) and Pd/C (122.79 mg 10%) in MeOH (4 mL) at room temperature. The resulting mixture was stirred for overnight at room temperature under hydrogen atmosphere. The resulting mixture was filtered, the filter cake was washed with MeOH (3×10 mL). The resulting mixture was concentrated under reduced pressure to afford 300b (397 mg, 94.85%) as light yellow solid.

### 3. Synthesis of 300c

A solution of 300b (397 mg, 0.814 mmol, 1 equiv) in DCE (4 mL) was treated with 330-2 (258.50 mg, 0.896 mmol, 1.1 equiv) for 2h at room temperature under nitrogen atmosphere followed by the addition of STAB (259.16 mg, 1.222 mmol, 1.5 equiv) at room temperature. The resulting mixture was stirred for overnight at room temperature. The reaction was quenched with sat. NaHCO₃ (aq.)(8 ml) at room temperature. The resulting mixture was extracted with DCE/MeOH (10:1) (3×20 mL). The combined organic layers were concentrated under reduced pressure. The residue was purified by reverse flash chromatography with the following conditions: column, C18 silica gel; mobile phase A: Water (0.1% NH4HCO3); mobile phase B: ACN, Gradient: 20% B to 80% B in 30 min; detector, UV 254 nm. This resulted in 300c (310 mug, 73.47%) as a yellow solid,

### 4. Synthesis of 300

A solution of 300c (300 mg, 0,596 mmol, 1 equiv) in DCM (9 mL) was treated with Pyridine (282.73 mg, 3.576 mmol, 6 equiv) at room temperature followed by the addition of Triphosgene (70.71 mg, 0.238 mmol, 0.4 equiv) at 0° C. The resulting mixture was stirred for 30 min at room temperature. The reaction was quenched by the addition of NaHCO₃ (15 mL) at room temperature. The resulting mixture was extracted with DCE/MeOH (10:1)(2×15 mL). After filtration, the filtrate was concentrated under reduced pressure. The residue was purified by reverse flash chromatography with the following conditions: column, C18 silica gel; mobile phase, A: Water (0.1% NH4HCO3), Mobile Phase B: ACN, Gradient: 20% B to 70% B in 30 min; UV 254 nm. This resulted in 300 (280 mg, 86.46%) as a yellow solid.

LC-MS: (ES, m/z): [M+H]+ 544. H-NMR: (400 MHz, DMSO, ppm): δ 1.03-1.05 (d, 3H), δ 1.65-1.90 (m, 6H), δ 2.03-2.08 (m, 2H), δ 2.65-2.75 (m, 2H), δ 3.24-3.28 (m, 3H), δ 3.46-3.52 (m, 2H), δ 3.73-3.75 (d, 1H), δ 4.71-4.74 (d, 1H), δ 7.02 (s, 1H), δ 7.33-7.38 (m, 2H), δ 7.46-7.50 (m, 1H), 7.70 (s, 1H), δ 7.75-7.77 (m, 1H), δ 7.86 (s, 1H), δ 9.50 (s, 1H).

### Example 301. Synthesis of Compound 301

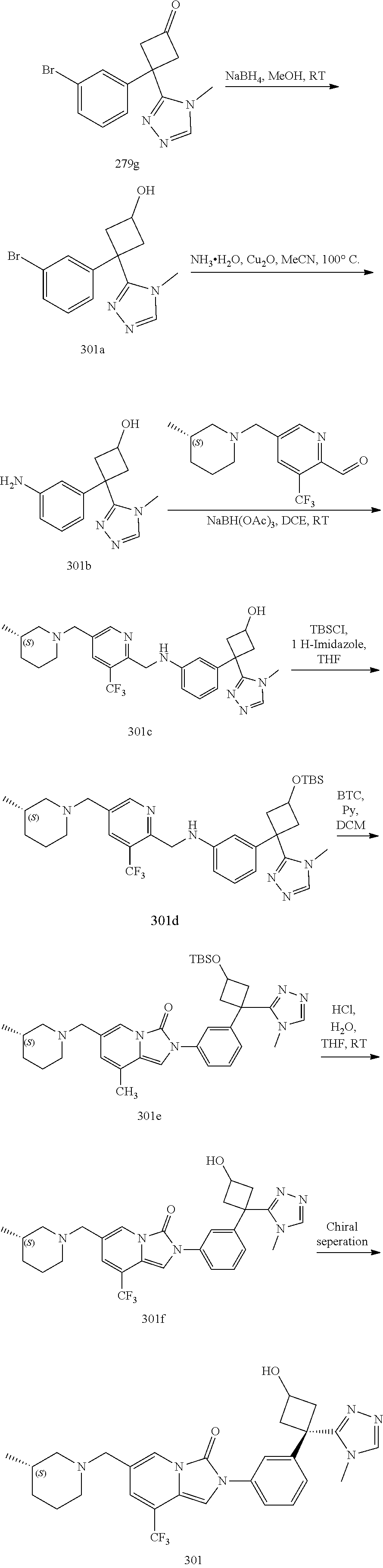

### 1. Synthesis of 301a

To a stirred solution of 279g (1 g, 3.266 mmol, 1 equiv) in MeOH (20 mL) was added NaBH4 (0.25 g, 6.532 mmol, 2 equiv) at room temperature. The resulting mixture was stirred for 2h at room temperature. The reaction was quenched with saturated NH4Cl (aq.) (100 mL) at room temperature. The aqueous layer was extracted with EtOAc (×30 mL). The resulting mixture was concentrated under vacuum. This resulted in 301a (1 g, 91.40%) as an off-white solid.

### 2. Synthesis of 301b

A solution of 301a (1 g, 3.245 mmol, 1 equiv) and N13·H2 O (8 mL) in MeCN (8 mL) was stirred overnight at 110° C. in a pressure tank. The mixture was allowed to cool down to room temperature. The resulting mixture was concentrated under reduced pressure. The residue was purified by Prep-TLC (DCM/MeOH 10:1) to afford 301b (0.6 g, 68.12%) as a yellow solid.

### 3. Synthesis of 301c

To a stirred solution of 301b (600 mg, 2.456 mmol, 1 equiv) and 12 (914.10 mg, 3.193 mmol, 1.3 equiv) in DCE (8 mL) was added NaBH(OAc)3 (1041.06 mg, 4.912 mmol, 2 equiv) at room temperature. The resulting mixture was stirred overnight at room temperature. The reaction was quenched with saturated NH4Cl (aq.) (50 mL) at room temperature. The aqueous layer was extracted with DCM (30 mL). The resulting mixture was concentrated under vacuum. The resulting mixture was concentrated under reduced pressure. The residue was purified by Prep-TLC (DCM/MeOH 20:1) to afford 301c (600 mg, 42.73%) as a yellow solid.

### 4. Synthesis of 301d

To a stirred solution of 301c (600 mg, 1,166 mmol, 1 equiv) and 1H-imidazole (317.50 mg, 4.664 mmol, 4 equiv) in THF (20 mL) was added TBSCl (439.33 mg, 2.915 mmol, 2.5 equiv) at room temperature. The resulting mixture was stirred overnight at 60° C. The resulting mixture was diluted with water (100 mL). The aqueous layer was extracted with EtOAc (2×50 mL). The resulting mixture was concentrated under vacuum. The residue was purified by Prep-TLC (DCM/MeOH 20:1) to afford 301d (500 mg, 64.78%) as a light yellow solid.

### 5. Synthesis of 301e

To a stirred solution of 301d (500 mg, 0.795 mmol, 1 equiv) and Pyridine (628.92 mg, 7,950 mmol, 10 equiv) in DCM (10 mL) was added Triphosgene (117.96 mg, 0,398 mmol, 0.5 equiv) at room temperature. The resulting mixture was stirred for 10 min at room temperature. The reaction was quenched with saturated NaHCO₃ (aq.) (50 mL) at room temperature. The aqueous layer was extracted with DCM (2×50 mL). The resulting mixture was concentrated under vacuum. This resulted in 301e (400 mg, 69.91%) as a yellow solid. The crude product was used in the next step directly without further purification.

### 6. Synthesis of 301f

To a stirred solution of 301e (400 mg, 0.611 mmol, 1 equiv) and H2 O (10 mL) in THE (5 mL) were added HCl (2 mL, 1M) at room temperature. The resulting mixture was stirred for 3h at room temperature. The mixture was neutralized to pH 7 with saturated NaHCO₃ (aq.). The aqueous layer was extracted with DCM (2×100 mL). The resulting mixture was concentrated under vacuum. The residue was purified by Prep-TLC (DCM/MeOH 8:1) to afford 301f (280 mg, 81.40%) as a yellow solid.

### 7. Synthesis of 301

301f (280 mg) was purified by Prep-HPLC with the following conditions (Column: CHIRALPAK IG, 2*25 cm, 5 μm; Mobile Phase A: Hex (0.5% 2M NH3-MeOH)-HPLC, Mobile Phase B: EtOH:DCM=1:1; Flow rate: 20 mL/min; Gradient: 25% B to 25% B in 21 min; Wave Length: 220/254 nm; RT1 (min): 12.10; RT2 (min): 16.72; the first peak is product) to afford 301 (105.8 mg, 36.77%) as a yellow solid.

LC-MS: (ES, m/z): [M+H]+ 541. H-NMR: (400 MHz, DMSO-d6, δ ppm): 0.81-0.93 (m, 4H), 1.42-1.57 (m, 1H), 1.57-1.73 (m, 4H), 1.85-1.91 (m, 1H), 2.49-2.51 (m, 1H), 2.51-2.53 (m, 1H), 3.20-3.30 (m, 7H), 4.04-4.09 (m, 1H), 5.32-5.33 (d, 1H), 7.00 (s, 1H), 7.07-7.09 (m, 1H), 7.36 (s, 1H), 7.45-7.49 (m, 1H), 7.63-7.68 (m, 3H), 8.37 (s, 1H).

### Example 302. Synthesis of Compound 302

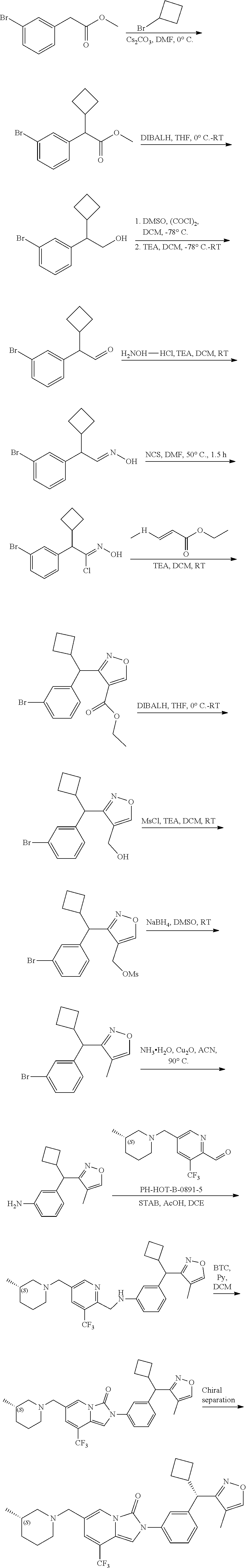

### 1. Synthesis of 302a

Into a 1 L 3-necked round-bottom flask were added methyl 2-(3-bromophenyl)acetate (30 g 130.963 mmol, 1 equiv), DMF (400 ml), Cs2CO3 (213.35 g, 654.815 mmol, 5 equiv) and bromocyclobutane (53.04 g, 392.889 mmol, 3 equiv) at 0° C. The resulting mixture was stirred for overnight at room temperature. The reaction was quenched by the addition of sat. NH4Cl (aq.) (1000 mL) at room temperature. The aqueous layer was extracted with EtOAc (3×500 mL). The resulting mixture was concentrated under reduced pressure. The residue was purified by silica gel column chromatography eluted with PE/EA (12:1) to afford 302a (15 g, 40.45%) as a light yellow oil.

### 2. Synthesis of 302b

Into a 500 ml, 3-necked round-bottom flask were added 302a (15 g, 52.973 mmol, 1 equiv), THF (200 m-L) and DIBALH (18.83 g, 132.433 mmol, 2.5 equiv) at 0° C. The resulting mixture was stirred for overnight at room temperature. The reaction was quenched by the addition of sat. NH4Cl (aq.) (500 mL) at room temperature. The aqueous layer was extracted with EtOAc (3×300 mL). The resulting mixture was concentrated under reduced pressure. The residue was purified by silica gel column chromatography eluted with PE/EA (10:1) to afford 302b (10 g, 73.99%) as a light yellow oil.

### 3. Synthesis of 302c

Into a 250 ml 3-necked round-bottom flask were added 302b (10 g, 39,192 mmol, 1 equiv), DCM (200 mL) and DMSO (0.31 g, 3,919 mmol, 0.1 equiv) at −78° C. The resulting mixture was stirred for 0.5 h at −78° C. The resulting mixture was concentrated under reduced pressure. To a stirred solution DCM (200 mL) and TEA (7.93 g, 78.384 mmol, 2 equiv) was added at −78° C. The resulting mixture was stirred for 2 h at room temperature. The reaction was quenched by the addition of Water (150 mL) at room temperature. The aqueous layer was extracted with EtOAc (3×200 mL). The resulting mixture was concentrated under reduced pressure. The residue was purified by silica gel column chromatography, eluted with PE/EA (10:1) to afford 302c (5 g, 50.40%) as a light brown oil.

### 4. Synthesis of 302d

Into a 250 mL, 3-necked round-bottom flask were added 302c (5 g, 19.752 mmol, 1 equiv), DCM (60 ml), TEA (6.00 g, 59,256 mmol, 3 equiv) and NH₂OH·HCl (3.43 g, 49.380 mmol, 2.5 equiv) at room temperature. The resulting mixture was stirred for overnight at room temperature. The reaction was quenched by Water (100 mL) at room temperature. The aqueous layer was extracted with EtOAc (3×50 mL). The resulting mixture was concentrated under reduced pressure. The residue was purified by silica gel column chromatography, eluted with PE/EA (5:1) to afford 302d (3.3 g, 62.30%) as a light yellow oil.

### 5. Synthesis of 302e

Into a 250 mL 3-necked round-bottom flask were added 302d (3.3 g, 12,306 mmol, 1 equiv), DMF (50 mL) and NCS (2.46 g, 18.459 mmol, 1.5 equiv) at room temperature. The resulting mixture was stirred for 1.5h at 50° C. The reaction was quenched by the addition of Water (150 mL) at room temperature. The aqueous layer was extracted with EtOAc (3×100 mL). The resulting mixture was concentrated under reduced pressure to afford 302e (3.5 g, 93.99%) as a light yellow oil.

### 6. Synthesis of 302f

Into a 250 mL 3-necked round-bottom flask were added 302e (3.8 g, 12.558 mmol, 1 equiv), DCM (40 mL), TEA (3.81 g, 37,674 mmol, 3 equiv) and ethyl (2E)-3-(dimethylamino)prop-2-enoate (2.70 g, 18,837 mmol, 1.5 equiv) at room temperature. The resulting mixture was stirred for overnight at room temperature. The reaction was quenched by the addition of Water (100 mL) at room temperature. The aqueous layer was extracted with EtOAc (3×50 mL). The resulting mixture was concentrated under reduced pressure. The residue was purified by silica gel column chromatography eluted with PE/EA (10:1) to afford 302f (2.4 g, 52.47%) as a light yellow oil.

### 7. Synthesis of 302g

Into a 250 mL 3-necked round-bottom flask were added 302f (2.4 g, 6.589 mmol, 1 equiv), THF (30 mL) and DIBALH (2.34 g, 16.473 mmol, 2.5 equiv) at 0° C. The resulting mixture was stirred for overnight at room temperature. The reaction was quenched by the addition of sat. NH4Cl (aq.) (150 mL) at room temperature. The aqueous layer was extracted with EtOAc (3×50 mL). The resulting mixture was concentrated under reduced pressure. The residue was purified by silica gel column chromatography eluted with PE/EA (10:1) to afford 302g (2 g, 94.21%) as a light yellow oil.

### 8. Synthesis of 302h

Into a 100 ml-3-necked round-bottom flask were added 302g (1.9 g, 5.897 mmol, 1 equiv), DCM (20 mL), TEA (1.79 g, 17.691 mmol, 3 equiv) and MsCl (1.01 g, 8.846 mmol, 1.5 equiv) at room temperature. The resulting mixture was stirred for 2 b at room temperature. The reaction was quenched by the addition of Water (50 mL) at room temperature. The aqueous layer was extracted with EtOAc (3×30 mL). The resulting mixture was concentrated under reduced pressure to afford 302h (2.1 g, 88.97%) as a light yellow oil.

### 9. Synthesis of 302i

Into a 100 ml-3-necked round-bottom flask were added 302h (2 g, 4.996 mmol, 1 equiv), DMSO (25 mL) and NaBH4 (0.378 g, 9.992 mmol, 2 equiv) at room temperature. The resulting mixture was stirred for 2 h at room temperature. The reaction was quenched by the addition of sat. NH4Cl (aq.) (50 mL) at room temperature. The aqueous layer was extracted with EtOAc (3×30 mL). The resulting mixture was concentrated under reduced pressure. The residue was purified by silica gel column chromatography eluted with PE/EA (5:1) to afford 302i (1 g, 65.42%) as a light yellow solid.

### 10, Synthesis of 302j

Into a 50 mL sealed tube were added 302i (400 mg, 1.306 mmol, 1 equiv), ACN (4 mL), NH3·H2 O (4 mL) and Cu2O (37.38 mg, 0,261 mmol, 0.2 equiv) at room temperature. The resulting mixture was stirred for overnight at 100° C. under nitrogen atmosphere. The resulting mixture was filtered; the filter cake was washed with MeCN (3×3 mL). The filtrate was concentrated under reduced pressure. The residue was purified by Prep-TLC eluted with CH2Cl2/MeOH (10:1) to afford 302j (290 mg, 91.61%) as a light yellow solid.

### 11. Synthesis of 302k

Into a 100 mL sealed tube were added 302j (290 mg, 1,197 mmol, 1 equiv), DCE (5 mL), 1-2 (342.63 mg, 1,197 mmol, 1 equiv), AcOH (71.87 mg, 1,197 mmol, 1 equiv) and STAB (507.28 mg, 2.394 mmol, 2 equiv) at room temperature. The resulting mixture was stirred for overnight at room temperature. The reaction was quenched by the addition of sat. NH4Cl (aq.) (25 mL) at room temperature. The aqueous layer was extracted with EtOAc (3×15 mL). The resulting mixture was concentrated under reduced pressure. The residue was purified by Prep-TLC eluted with CH2Cl2/MeOH (12:1) to afford 302k (500 mg, 81.50%) as a light yellow solid.

### 12. Synthesis of 3021

Into a 100 mL sealed tube were added 302k (480 mg, 0.936 mmol, 1 equiv), DCM (10 mL), Pyridine (444.40 mg, 5.616 mmol, 6 equiv) and Triphosgene (111.15 mg, 0.374 mmol, 0.4 equiv) at 0° C. The resulting mixture was stirred for 10 min at room temperature under nitrogen atmosphere. The reaction was quenched by the addition of sat. NaHCO3 (aq.) (20 mL) at room temperature. The aqueous layer was extracted with CH2Cl2 (3×10 mL). The resulting mixture was concentrated under reduced pressure. The residue was purified by Prep-TLC eluted with CH2Cl2/MeOH (10:1) to afford 3021 (200 mg, 39.66%) as a light yellow solid.

### 13. Synthesis of 302

The 3021 (200 mg) was purified by Prep-HPLC with the following conditions (Column: CHIRALPAK IF, 2*25 cm, 5 μm; Mobile Phase A: Hex (0.5% 2M N13-MeOH)-HPLC, Mobile Phase B: EtOH-HPLC; Flow rate: 20 mL/min; Gradient: 5% B to 5% B in 20 min; Wave Length: 220/254 nm; RT1 (min): 13.98; RT2 (min): 17.69; The first peak was the product. Injection Volume: 0.65 mL; Number of Runs: 15) to afford 302 (43.6 mg, 21.08%) as a light yellow solid.

H-NMR: 1H NMR (300 MHz, DMSO-d4) δ 0.82-0.84 (m, 4H), 1.58 (s, 1H), 1.77-1.80 (m, 5H), 1.81-1.92 (m, 4H), 1.93-1.99 (m, 4H), 2.10 (s, 1H), 2.61-2.81 (m, 2H), 3.02-3.13 (m, 1H), 3.24-3.32 (m, 2H), 4.14-4.16 (d, 1H), 7.00 (s, 1H), 7.21-7.23 (d, 1H), 7.32 (s, 1H), 7.40-7.48 (m, 1H), δ 7.55-7.70 (m, 2H), 7.76 (s, 1H), 8.52 (s, 1H).

### Example 303. Synthesis of Compound 303

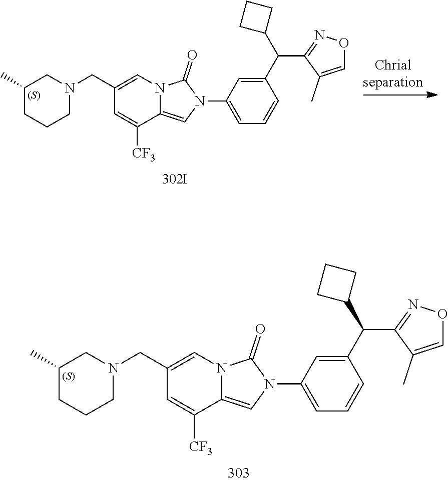

### Synthesis of 303

302l (200 mg) was purified by Prep-HPLC with the following conditions (Column: CHIRALPAK IF, 2*25 cm, 5 μm; Mobile Phase A: Hex (0.5% 2M NH3-MeOH)-HPLC, Mobile Phase B: EtOH-PLC; Flow rate: 20 mL/min; Gradient: 5% B to 5% B in 20 min; Wave Length: 220/254 nm; RT1 (min): 13.98; RT2 (min): 17.69; The second peak was the product. Injection Volume: 0.65 mL; Number of Runs: 15) to afford 303 (52.6 mg, 25.12%) as a light yellow solid.

H-NMR: 1H NMR (300 MHz, DMSO-d6) δ 0.83-0.85 (m, 4H), 1.59 (s, 1H), 1.77-1.80 (m, 5H), 1.81-1.92 (m, 4H), 1.93-1.99 (m, 4H), 2.10 (s, 1H), 2.61-2.81 (m, 2H), 3.02-3.13 (m, 1H), 3.24-3.32 (m, 2H), 4.14-4.16 (d, 1H), 7.00 (s, 1H), 7.21-7.23 (d, 1H), 7.32 (s, 1H), 7.40-7.48 (m, 1H), δ 7.55-7.70 (m, 2H), 7.76 (s, 1H), 8.53 (s, 1H).

### Example 304. Synthesis of Compound 304

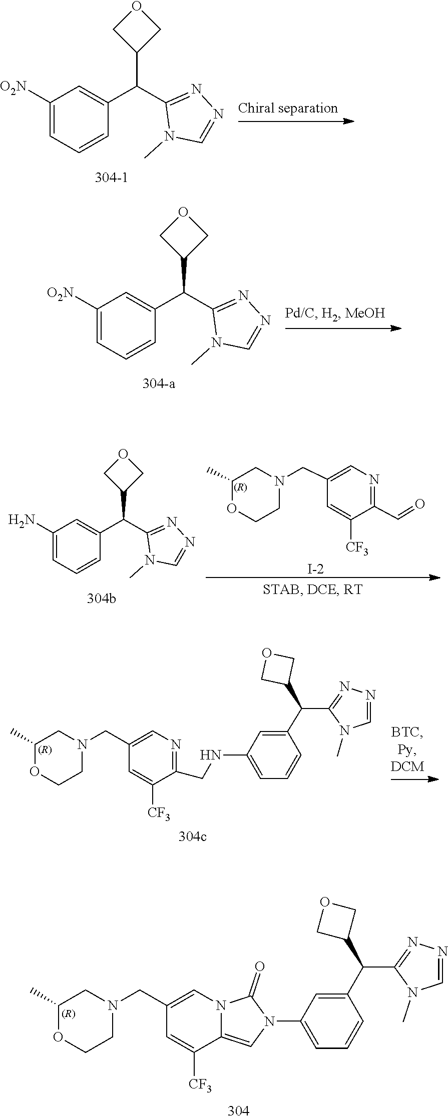

### 1. Synthesis of 304a

The 304-1 (11.5 g) was purified by Prep-CHIRAL-HPLC with the following conditions (Column: Lux 5 um Cellulose-4, 5*25 cm, 10 μm; Mobile Phase A: C02, Mobile Phase B: MeOH: ACN=2:1 (0.1% 2M NH3-MeOH); Flow rate: 200 mL/min; Gradient: isocratic 50% B; Column Temperature (° C.): 35; Back Pressure (bar): 100; Wave Length: 254 nm; RT1 (min): 4.25; RT2 (min): 9.57; The second peak was product) to afford 304a (5 g, 43%) as a yellow solid.

### 2. Synthesis of 304b

To a solution of 304a (5 g, 18.23 mmol, 1.0 equiv) in 150 mL MeOH was added Pd/C (10%, 1g) under nitrogen atmosphere in a 250 mL round-bottom flask. The mixture was hydrogenated at room temperature for overnight under hydrogen atmosphere using a hydrogen balloon, filtered through a Celite pad and concentrated under reduced pressure. This resulted in 304b (4.2 g, 92%) as an off-white solid.

### 3. Synthesis of 304c

Into a 50 mL 3-necked round-bottom flask were added 304b (300 mg, 1.228 mmol, 1 equiv), STAB (520.53 mg, 2.456 mmol, 2 equiv) and DCE (4 mL) at room temperature. The resulting mixture was stirred for 2h at room temperature. The reaction was quenched by the addition of sat. NH4Cl (aq.) (10 mL) at room temperature. The aqueous layer was extracted with CH2Cl2 (2×10 mL). The resulting mixture was concentrated under vacuum. The residue was purified by Prep-TLC (CH2Cl2/MeOH 20:1) to afford 304c (320 mg, 50.45%) as a yellow solid.

### 4. Synthesis of 304

Into a 50 mL 3-necked round-bottom flask were added 304c (310 mg, 0.600 mmol, 1 equiv), Pyridine (284.81 mg, 3.600 mmol, 6 equiv) and DCM (4 mL) at room temperature. To the above mixture was added Triphosgene (89.04 mg, 0.300 mmol, 0.5 equiv) at 0° C. The resulting mixture was stirred for additional 30 min at room temperature. The reaction was quenched by the addition of sat. NaHCO₃ (aq.) (10 mL) at room temperature. The aqueous layer was extracted with CH2Cl2 (3×15 mL). The resulting mixture was concentrated under vacuum. The residue was purified by Prep-TLC (CH2Cl2/MeOH 20:1) to afford the crude product. The crude product was purified by reverse phase flash with the following conditions: (column, C18; mobile phase, A: water (10 mmol/L N1411C03), B: CH3CN, 5% B to 75% B gradient in 30 min; detector, UV 254 nm) to afford 304 (157.8 mg, 48.17%) as a yellow solid.

H-NMR: 61.03 (s, 3H), δ 1.71-1.76 (m, 1H), δ 2.04-2.07 (m, 1H), δ 2.65-2.68 (m, 1H), δ2.72-2.75 (m, 1H), δ 3.23 (s, 2H), δ 3.26-3.31 (m, 3H), δ 3.46-3.51 (m, 2H), δ 3.73-3.75 (m, 1H), δ 3.91-3.94 (m, 1H), δ 4.27-4.30 (t, 1H), δ 4.48-4.50 (d, 2H), δ 4.73-4.82 (m, 2H), δ 7.02 (s, 1H), δ 7.17-7.19 (d, 1H), δ 7.39 (s, 1H), δ 7.45-7.49 (m, 1H), δ 7.68 (s, 1H), δ 7.75-7.77 (d, 1H), δ 8.29 (s, 1H).

### Example 305. Synthesis of Compound 305

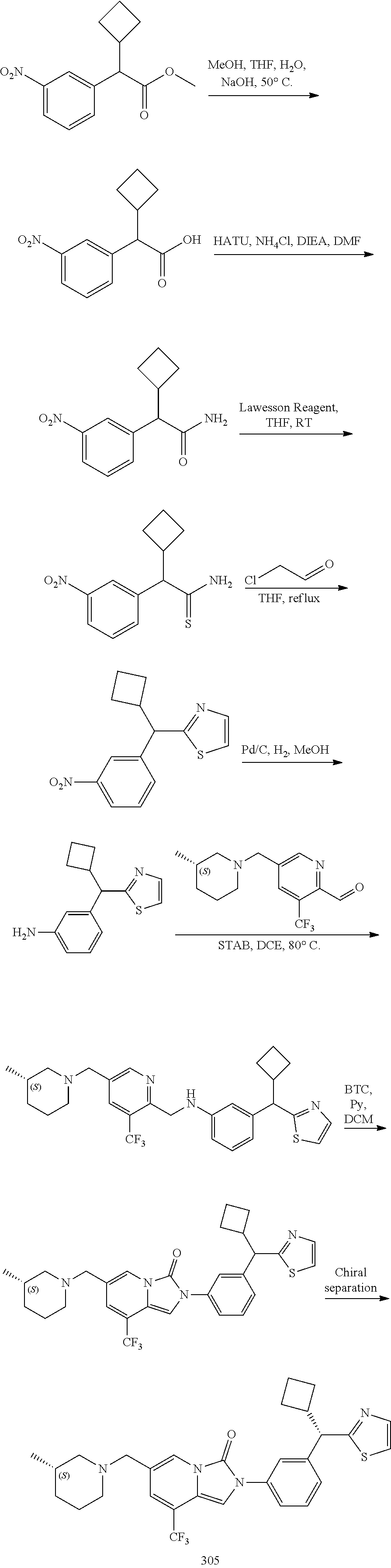

### 1. Synthesis of 305a

Into a 500 mL 3-necked round-bottom flask were added methyl 2-cyclobutyl-2-(3-nitrophenyl)acetate (25 g, 100,294 mmol, 1 equiv), MeOH (120 mL), THF (40 mL), H120 (40 mL) and NaOH (8.02 g, 200,588 mmol, 2 equiv) at room temperature. The resulting mixture was stirred for 30 min at 50° C. The mixture was acidified to pH 4 with HC (1M). The precipitated solids were collected by filtration and washed with water (2×100 mL). The resulting mixture was concentrated under vacuum. This resulted in 305a (23 g, 92.61%) as a white solid.

### 2, Synthesis of 305b

Into a 500 mL 3-necked round-bottom flask were added 205a (23 g, 97.773 mmol, 1 equiv), DMF (230 mL) HATU (55.77 g, 146.659 mmol, 1.5 equiv), NH4Cl (10.46 g, 195.546 mmol, 2 equiv) and DI EA (25.27 g, 195.546 mmol, 2 equiv) at room temperature. The resulting mixture was stirred for overnight at room temperature. The mixture was basified to pH 7 with saturated NaHCO₃ (aq.). The resulting mixture was extracted with EtOAc (3×300 mL). The resulting mixture was concentrated under vacuum. The residue was purified by silica gel column chromatography, eluted with PE/EA (5:1) to afford 205b (20 g, 82.96%) as a light yellow solid.

### 3. Synthesis of 305c

Into a 500 mL 3-necked round-bottom flask were added 305b (20 g, 85.377 mmol, 1 equiv), THE (200 mL) and Lawesson Reagent (41.44 g, 102,452 mmol, 1.2 equiv) at room temperature. The resulting mixture was stirred for overnight at room temperature. The reaction was quenched with Water (500 ml) at room temperature. The resulting mixture was extracted with EtOAc (3×500 mL). The resulting mixture was concentrated under vacuum. The residue was purified by silica gel column chromatography, eluted with PE/EA (10:1) to afford 305c (10 g, 44.45%) as a light yellow solid.

### 4. Synthesis of 305d

Into a 250 mL 3-necked round-bottom flask were added 305c (10 g, 39.949 mmol, 1 equiv), THF (100 mL) and chloroacetaldehyde (4.70 g, 59.923 mmol, 1.5 equiv) at room temperature. The resulting mixture was stirred for overnight at 60° C. under nitrogen atmosphere. The reaction was quenched by the addition of Water (200 mL) at room temperature. The resulting mixture was extracted with EtOAc (3×300 mL). The combined organic layers were washed with water (100 mL), dried over anhydrous Na2SO4. After filtration, the filtrate was concentrated under reduced pressure. The residue was purified by silica gel column chromatography, eluted with PE/EA (10:1) to afford 305d (7 g, 63.23%) as a light yellow oil.

### 5. Synthesis of 305e

To a solution of 305d (7 g, 25.516 mmol, 1 equiv) in 150 mL MeOH was added Pd/C (20%, 1.4 g) under nitrogen atmosphere in a 250 mL 3-necked round-bottom flask. The mixture was hydrogenated at room temperature for overnight under hydrogen atmosphere using a hydrogen balloon, filtered through a Celite pad and concentrated under reduced pressure to afford 305e (6 g, 91.42%) as a light yellow oil.

### 6. Synthesis of 305f

Into a 100 ml 3-necked round-bottom flask were added 305e (2 g, 8.185 mmol, 1 equiv), DCE (20 mL), STAB (5.20 g, 24.555 mmol, 3 equiv) and I-2 (3.05 g, 10.641 mmol, 1.3 equiv) at room temperature. The resulting mixture was stirred for overnight at 80° C. The reaction was quenched with sat. NH4Cl (aq.) (30 ml) at room temperature. The resulting mixture was extracted with CH2Cl2 (3×30 mL). The resulting mixture concentrated under reduced pressure. The residue was purified by silica gel column chromatography, eluted with CH2Cl2/MeOH (30:1) to afford 305f (2.9 g, 65.40%) as a light yellow oil,

### 7. Synthesis of 305g

Into a 100 ml round-bottom flask were added 305f (600 mg, 1.166 mmol, 1 equiv), DCM (30 mL) and Pyridine (553.3 mg, 6.996 mmol, 6 equiv) at 0° C. To the above mixture was added Triphosgene (155.7 mg, 0.524 mmol, 0.45 equiv) at 0° C. The resulting mixture was stirred for additional 8 min at 0° C. The reaction was quenched by the addition of sat. NaHCO₃ (aq.) (10 mL) at room temperature. The resulting mixture was extracted with CH2Cl2 (3×20 mL). The resulting mixture was concentrated under reduced pressure. The residue was purified by Prep-TLC (CH2Cl2/MeOH 30:1) to afford 305g (340 mg, 51.78%) as a light yellow solid.

### 8. Synthesis of 305

305g (300 mg) was purified by Chiral separation with the following conditions (Column: CHIRALPAK IG, 2*25 cm, 5 um; Mobile Phase A: Hex (0.5% 2M NH3-MeOH)-HPLC, Mobile Phase B: EtOH:DCM=1:1-HPLC; Flow rate: 20 mL/min; Gradient: 10% B to 10% B in 18 min; Wave Length: 220/254 nm; RT (min): 9.34; RT2 (min): 13.32; The first peak was the product. Sample Solvent: EtOH:DCM=1:1-HPLC; Injection Volume: 1.1 mL; Number of Runs: 6) to afford 305 as a light yellow solid.

LCMS: (ES, m/z): [M+H]+ 541. H-NMR: (300 MHz, DMSO-d6, ppm): δ 0.827-0.867 (m, 4H), δ 1.47-1.66 (m, 5H), δ 1.66-1.84 (m, 6H), δ 2.04-2.06 (d, 1H), δ 2.50 (s, 1H), δ 2.51-2.75 (m, 2H), δ 3.34 (s, 2H), δ 4.48-4.50 (d, 1H), δ 7.01 (s, 1H), δ 7.32-7.34 (d, 2H), δ 7.58-7.59 (d, 1H), δ 7.66 (s, 1H), δ 7.71 (s, 1H), δ 7.72-7.73 (d, 2H), δ 7.85 (s, 1H).

### Example 306. Synthesis of Compound 306

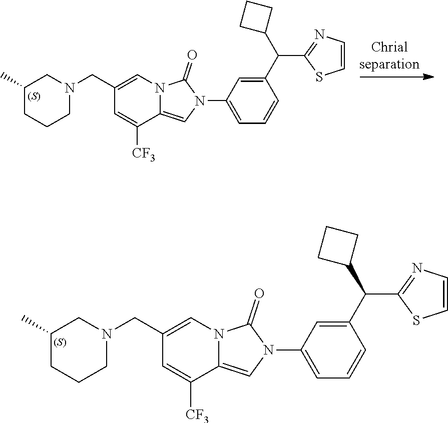

### Synthesis of 306

The crude product (300 mg) was purified by Chiral separation with the following conditions (Column: CHIRALPAK IG, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 10% B to 10% B in 18 min; Wave Length: 220/254 nm; RT1 (min): 9.34; RT2 (min): 13.32; The second peak was the product. Sample Solvent: EtOH:DCM=1: I-HPLC; Injection Volume: 1.1 mL; Number of Runs: 6) to afford 306 as a light yellow solid.

LCMS (ES, m/z): [M+H]+ 541. H-NMR0: (300 MHz, DMSO-d6, ppm): δ 0.82-0.83 (m, 4H), δ 1.58-1.63 (m, 6H), δ 1.81-1.83 (m, 6H), δ 2.04-2.06 (d, 1H), δ 2.75 (d, 2H), δ 3.25 (s, 2H), δ 4.48-4.50 (d, 1H), δ 7.01 (s, 1H), δ 7.32-7.34 (d, 2H), δ 7.45 (s, 1H), δ 7.58-7.59 (s, 1H), δ 7.66 (s, 1H), δ 7.71-7.73 (m, 2H), δ 7.85 (s, 1H).

### Example 307. Synthesis of Compound 307

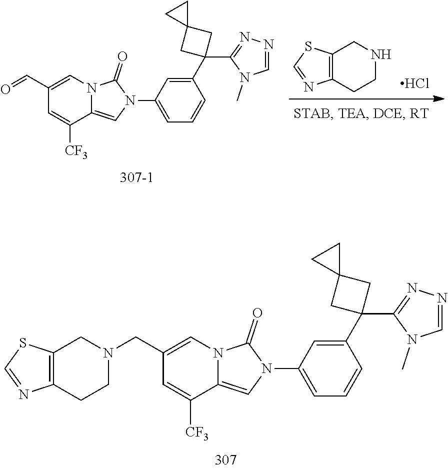

### Synthesis of 307

To a stirred solution of 307-1 (400 mg, 0.856 mmol, 1 equiv) and 41.5H,6H,7H-[1,3]thiazolo[5,4-c]pyridine hydrochloride (226.75 mg, 1.284 mmol, 1.5 equiv) in DCE (5 mL) was added TEA (129.89 mg, 1.284 mmol, 1.5 equiv) at room temperature. The resulting mixture was stirred for 1b at room temperature. To the above mixture was added STAB (362.72 mg, 1.712 mmol, 2 equiv) at room temperature. The resulting mixture was stirred overnight at room temperature. The reaction was quenched with saturated NH4Cl (aq.) (50 nil) at room temperature. The aqueous layer was extracted with DCM (2×30 mL). The resulting mixture was concentrated under vacuum. The residue was purified by Prep-TLC (DCM/MeOH 18:1) to afford 307 (180.7 mg, 35.05%) as a yellow solid.

LC-MS: (ES, m/z): [M+H]+ 592. H-NMR: (400 MHz, DMSO-d6, δ ppm): 0.48-0.56 (m, 2H), 0.57-0.60 (m, 2H), 2.75-2.78 (m, 2H), 2.75-2.82 (m, 2H), 2.82-2.86 (m, 2H), 3.18-3.22 (m, 2H), 3.25 (s, 3H), 3.53 (s, 2H), 3.75 (s, 2H), 7.07 (s, 1H), 7.19-7.21 (d, 1H), 7.36 (s, 1H), 7.49-7.53 (m, 1H), 7.64-7.66 (m, 1H), 7.76 (s, 1H), 7.83 (s, 1H), 8.40 (s, 1H), 8.91 (s, 1H).

### Example 308. Synthesis of Compound 308

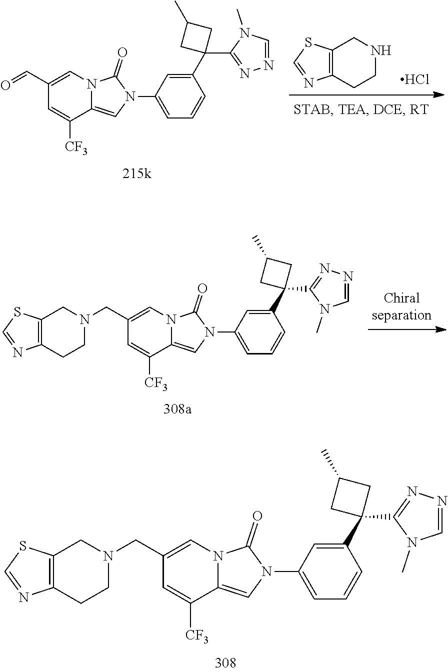

### 1. Synthesis of 308a

To a stirred mixture of 4-H,5H,6H,7H-[1,3]thiazolo[5,4-c]pyridine hydrochloride (465.47 mg, 2,634 mmol, 2 equiv) and 215k (600 mg, 1.317 mmol, 10 equiv) in DCE (6 mL) was added TEA (399.94 mg, 3.951 mmol, 3 equiv) at room temperature.The resulting mixture was stirred for 30 min at room temperature. To the above mixture was added STAR (837.63 mg, 3.951 mmol, 3 equiv) at room temperature. The resulting mixture was stirred for additional 2 h at room temperature. The reaction was quenched by the addition of NaHCO₃ (aq.) (30 mL) at room temperature. The aqueous layer was extracted with EtOAc (2×20 mL). The resulting mixture was concentrated under reduced pressure. The residue was purified by Prep-TLC (CH2Cl2/MeOH=1.5:1) to afford 308a (380 mg, 47.27%) as a yellow solid.

### 2. Synthesis of 308

308a (380 mg) was purified by Prep-CHIRAL-HPLC with the following conditions (Column: CHIRALPAK AD-H, 2*25 cm, 5 μm; Mobile Phase A: Hex (0.5% 2M NH3-MeOH), Mobile Phase B: EtOH; Flow rate: 20 mL/min; Gradient: 50% B to 50% B in 16.5 min; Wave Length: 220/254 nm; RT1 (min): 9.78; RT2 (min): 12.55; the first peak is product) to afford the crude product (130 mg). The crude product (130 tug) was purified by Prep-HPLC with the following conditions (Column: XBridge Prep OBD C18 Column, 30*150 mm, 5 μm; Mobile Phase A: water (10 mmol/L NH4HCO3), Mobile Phase B: ACN; Flow rate: 60 mL/min; Gradient: 27% B to 57% B in 8 min, 57% B; Wave Length: 220 nm; RT1 (min): 7.85) to afford 308 (721 mg, 18.21%) as a yellow solid.

LCMS: (ES, m/z): [M+H]+ 580. H-NMR: (400 MHz, DMSO-d6, ppm, δ): 1.08 (s, 3H), 2.54-2.56 (m, 2H), 2.84-2.89 (m, 6H), 3.18 (s, 3H), 3.55 (s, 2H), 3.77 (s, 2H), 7.07 (s, 1H), 7.26-7.27 (d, 1H), 7.39 (s, 1H), 7.49-7.53 (t, 1H), 7.64-7.66 (m, 1H), 7.77-7.82 (m, 2H), 8.30 (s, 1H), 8.92 (s, 1H).

### Example 309. Synthesis of Compound 309

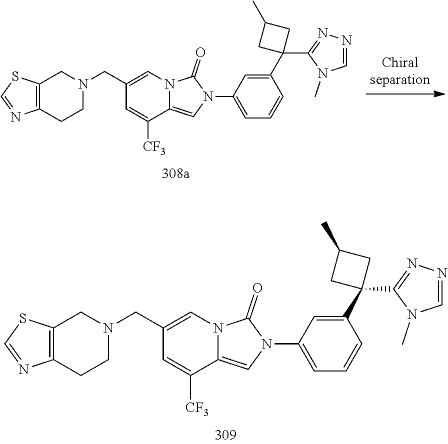 [SMILES: CC1CC(c2cccc(-n3cc4c(C(F)(F)F)cc(CN5CCc6ncsc6C5)cn4c3=O)c2)(c2nncn2C)C1 ; Cn1cnnc1[C@]1(c2cccc(-n3cc4c(C(F)(F)F)cc(CN5CCc6ncsc6C5)cn4c3=O)c2)C[C@@H](C)C1]

### Synthesis of 309

The crude product (380 mg) was purified by Prep-HPLC with the following conditions (Column: CHIRALPAK AD-H, 2*25 cm, 5 n; Mobile Phase A: Hex (0.5% 2M NH3-MeOH)-HPLC, Mobile Phase B: EtOH-HPLC; Flow rate: 20 mL/min; Gradient: 50% B to 50% B in 16.5 min; Wave Length: 220/254 nm; RT1 (min): 9.78; RT2 (min): 12.55; the second peak is product) to afford 309 (47.6 ng, 12.18%) as a yellow solid.

LCMS: (ES, m/z): [M+H]+ 580. H-NMR: (400 MHz, DMSO-d6, ppm, δ): 1.08-1.11 (d, 3H), 2.27-2.36 (m, 3H), 2.75-2.96 (m, 4H), 3.10-3.13 (m, 2H), 3.25 (s, 3H), 3.56 (s, 2H), 3.77 (s, 2H), 7.09-7.11 (m, 2H), 7.38 (s, 1H), 7.46-7.50 (t, 1H), 7.62-7.64 (m, 1H), 7.69-7.82 (m, 2H), 8.37 (s, 1H), 8.94 (s, 1H).

### Example 310. Synthesis of Compound 310

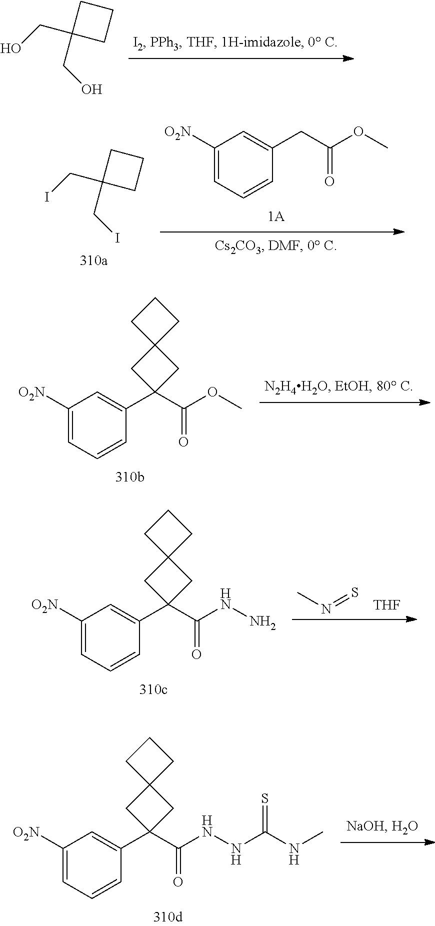

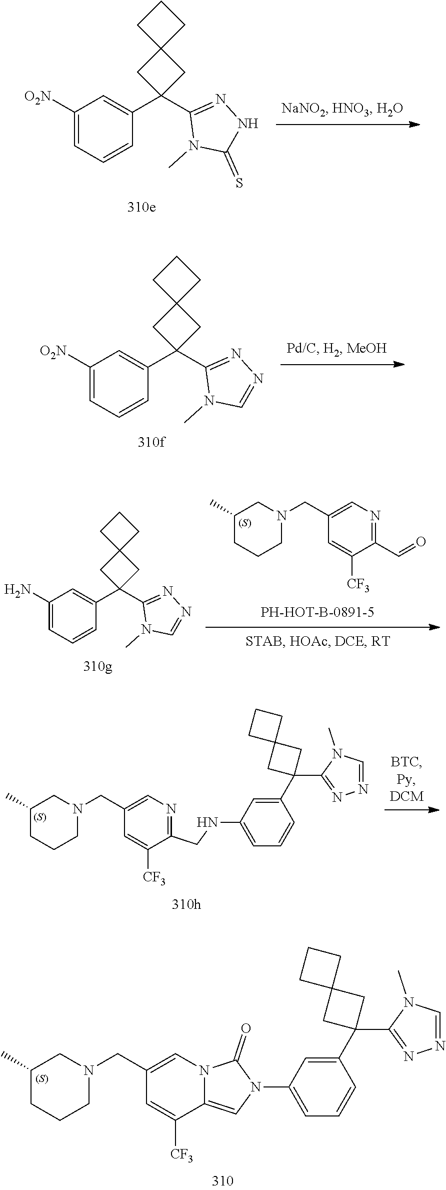

### 1. Synthesis of 310a

To a stirred solution of [1-(hydroxymethyl)cyclobutyl]methanol (9 g, 77.479 mmol, 1 equiv), PPh3 (26.42 g, 100.723 mmol, 1.3 equiv) and imidazole (7.38 g, 108.471 mmol, 1.4 equiv) in THF (90 mL) were added 12 (29.50 g, 116.219 mmol, 1.5 equiv) and at room temperature. The resulting mixture was stirred overnight at room temperature under nitrogen atmosphere. The resulting mixture was concentrated under reduced pressure. The residue was purified by silica gel column chromatography, eluted with PE/EA=100:1 to afford 310a (15 g, 57.63%) as a colorless oil.

### 2. Synthesis of 310b

To a stirred solution of methyl 2-(3-nitrophenyl)acetate (4.2 g, 21.519 mmol, 1.00 equiv) in DMF (150 mL) were added Cs2CO3 (35.06 g, 107.595 mmol, 5 equiv) at 0° C. under nitrogen atmosphere. The resulting mixture was stirred for 3 h at room temperature under nitrogen atmosphere. To the above mixture was added 310a (15.00 g, 44.652 mmol, 2.075 equiv). The resulting mixture was stirred for 24h at room temperature under nitrogen atmosphere. The resulting mixture was filtered, the filter cake was washed with EtOAc (100 mL). The filtrate was diluted with water (400 mL). The aqueous layer was extracted with EtOAc (2×200 mL). The resulting mixture was concentrated under reduced pressure. The residue was purified by silica gel column chromatography, eluted with PE/EA=50:1 to afford 310b (4.2 g, 70.89%) as a white solid.

### 3. Synthesis of 310c

To a stirred solution of 310b (4.2 g, 15.256 mmol, 1 equiv) in EtOH (42 mL) was added NH2NH2·H2 O (7.64 g, 152.560 mmol, 10 equiv) at room temperature. The resulting mixture was stirred for overnight at 80° C. The resulting mixture was diluted with water (300 mL). The aqueous layer was extracted with CH2Cl2:CH30H10:1 (3×100 mL). The combined organic layers were dried over Na2SO4. After filtration, the filtrate was concentrated under reduced pressure. This resulted in 310c (3.9 g, 92.86%) as a yellow solid.

### 4. Synthesis of 310d

To a stirred solution of 310c (3.9 g, 14.166 mmol, 1 equiv) in tetrahydrofuran (40 mL) was added methyl isothiocyanate (2.59 g, 35.415 mmol, 2.5 equiv) at room temperature. The resulting mixture was stirred for overnight at room temperature. The resulting mixture was diluted with water (40 mL). The THF was removed under reduced pressure. The precipitated solids were collected by filtration and washed with water. The resulting solid was dried under vacuum. This resulted in 310d (4.6 g, 93.20%) as a white solid.

### 5. Synthesis of 310e

To a stirred mixture of 310d (4.6 g, 13.202 mmol, 1 equiv) was added NaOH (1.84 g, in 45 mL H120) at room temperature. The resulting mixture was stirred for 4 h at room temperature. The mixture was neutralized to pH 6 with 1 M HCl (aq.). The precipitated solids were collected by filtration and washed with water (30 mL). The resulting solid was dried under vacuum. This resulted in afford 31 Ge (3 g, 68.77%) as a white solid.

### 6. Synthesis of 310f

To a stirred mixture of 310e (3 g, 9.080 mmol, 1 equiv) and NaNO2 (6.26 g, 90.800 mmol, 10 equiv) in Ethylacetate (14 mL) and H2O (52 mL) was added HNO3 (90.8 ml, 90.800 mmol, 10 equiv, 1 M) dropwise at 0° C. The resulting mixture was stirred for overnight at room temperature. The reaction was quenched by the addition of NaHCO3 (aq.) (200 mL) at room temperature. The aqueous layer was extracted with EtOAc (2×50 mL). The resulting mixture was concentrated under reduced pressure. The residue was purified by silica gel column chromatography, eluted with PE/EA=2:1 to afford 310f (2.7 g, 99.67%) as a white solid.

### 7. Synthesis of 310g

To a solution of 310f (2.7 g, 9.050 mmol, 1 equiv) in 50 mL MeOH was added Pd/C (10%, 0.54 g) under nitrogen atmosphere in a 250 mL round-bottom flask. The mixture was hydrogenated at room temperature for 1 h under hydrogen atmosphere by using a hydrogen balloon, filtered through a Celite pad and concentrated under reduced pressure to afford 310g (2.35 g, 96.76%) as a white solid.

### 8. Synthesis of 310h

To a stirred mixture of 310g (250 mg, 0.932 mmol, 1 equiv) and 1-2 (266.71 mg, 0.932 mmol, 1 equiv) in DCE (3 mL) was added HOAc (55.94 mg, 0.932 mmol, 1 equiv) and STAB (592.31 mg, 2,796 mmol, 3 equiv) at room temperature. The resulting mixture was stirred for 4 h at room temperature. The reaction was quenched by the addition of NaHCO₃ (30 mL) at room temperature. The aqueous layer was extracted with EtOAc (2×50 mL). The resulting mixture was concentrated under reduced pressure. The residue was purified by Prep-TLC (CH2Cl2/MeOH 15:1) to afford 310h (270 mg, 53.81%) as a yellow solid.

### 9. Synthesis of 310

To a stirred solution of 310h (250 mg, 0.464 mmol, 1 equiv) and pyridine (220.27 mg, 2,784 mmol, 6 equiv) in DCM (6 mL) was added Triphosgene (68.86 mg, 0.232 mmol, 0.5 equiv) at 0° C. The resulting mixture was stirred for 2 min at 0° C. The reaction was quenched by the addition of NaHCO₃ (aq.) (20 mL) at room temperature. The aqueous layer was extracted with CH2Cl2 (2×10 mL). The resulting mixture was concentrated under reduced pressure. The crude product was purified by reverse phase flash with the following conditions (Mobile Phase A: water (10 mmol/L NH4HCO3), Mobile Phase B: ACN; Flow rate: 30 ml/min; Gradient: 2% B to 100% B in 30 min) to afford the crude product (105 mg). The crude product was purified by Prep-TLC (CH2Cl2/MeOH=15:1) to afford 310 (75.4 mg, 27.76%) as a yellow solid.

LCMS: (ES, m/z): [M+H]+ 565. H-NMR: (400 MHz, DMSO-d6, ppm, δ): 0.80-0.95 (m, 4H), 1.40-1.74 (m, 5H), 1.74-1.80 (m, 2H), 1.86-1.93 (m, 3H), 1.97-2.01 (m, 2H), 2.73-2.80 (m, 4H), 3.05-3.09 (m, 2H), 3.25 (s, 5H), 7.00 (s, 1H), 7.13-7.15 (d, 1H), 7.34 (s, 1H), 7.45-7.49 (t, 1H), 7.59-7.62 (m, 1H), 7.66 (s, 1H), 7.77 (s, 1H), 8.32 (s, 1H).

### Example 311. Synthesis of Compound 311

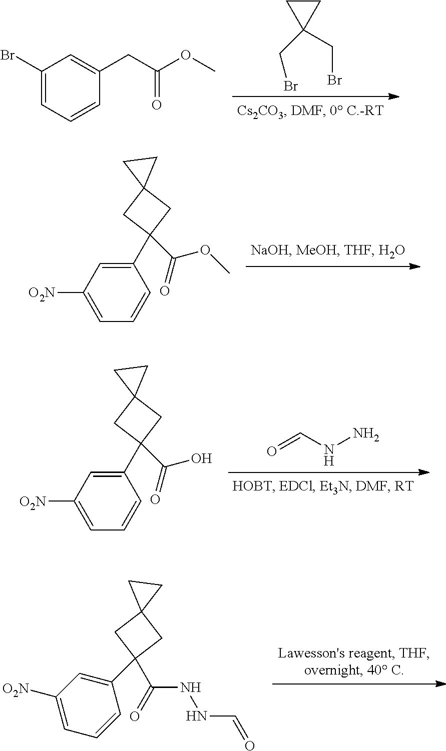

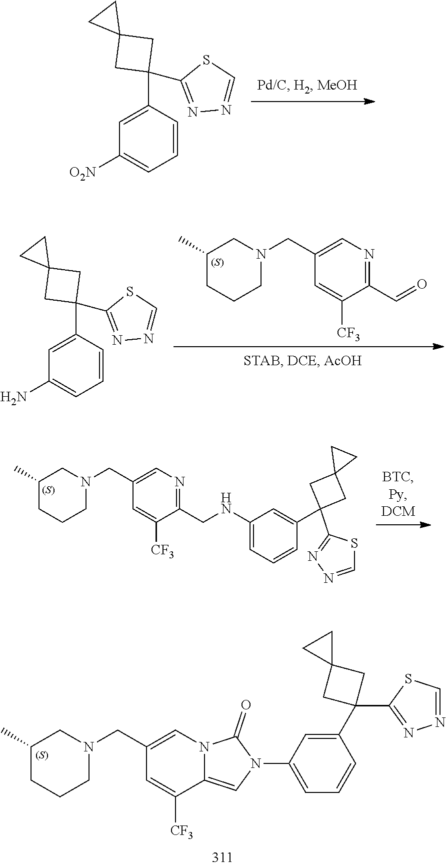

### 1. Synthesis of 311a

Into a 500 mL vial were added methyl 2-(3-nitrophenyl) acetate (20 g, 102,473 mmol, 1 equiv) 1,1-bis(bromomethyl)cyclopropane (70.07 g, 307.419 mmol, 3 equiv), Cs2CO3 (166.94 g 512.365 mmol, 5 equiv) and DMF (200 mL) at 0° C. The resulting mixture was stirred for overnight at room temperature under nitrogen atmosphere. The reaction was quenched with sat. NH4Cl (aq.) at room temperature. The aqueous layer was extracted with EtOAc (3×300 mL). The resulting mixture was concentrated under reduced pressure. The residue was purified by silica gel column chromatography, eluted with PE/EA (20:1) to afford 311a (13 g, 44.19%) as a yellow oil,

### 2. Synthesis of 311b

Into a 500 mL 3-necked round-bottom were added 311a (13 g, 49.756 mmol, 1 equiv), NaOH (5.97 g, 149,268 mmol, 3 equiv), MeOH (78 mL), THF (26 mL) and 1120 (26 mL) at room temperature. The resulting mixture was stirred for 30 min at 50° C. The reaction was quenched with HCl aq. (I M) at room temperature. The aqueous layer was extracted with EtOAc (3×300 mL). The resulting mixture was concentrated under reduced pressure. The residue was purified by silica gel column chromatography, eluted with PE/EA (10:1) to afford 311b (10 g, 75.60%) as a yellow oil.

### 3. Synthesis of 311c

Into a 500 mL 3-necked round-bottom were added 311b (10 g, 40.445 mmol, 1 equiv), N-formylhydrazine (7.29 g, 121.335 mmol, 3 equiv), HOBT (10.93 g, 80.890 mmol, 2 equiv), EDCI (23.26 g, 121.335 mmol, 3 equiv), Et3N (12.28 g, 121.335 mmol, 3 equiv) and DMF (100 mL) at room temperature. The resulting mixture was stirred for overnight at room temperature. The reaction was quenched with sat. NH4Cl (aq.) at room temperature. The aqueous layer was extracted with EtOAc (3×200 mL), and the resulting mixture was concentrated under reduced pressure. The residue was purified by silica gel column chromatography, eluted with PE/EA (5:1) to afford 311c (6 g, 47.69%) as a yellow oil.

### 4. Synthesis of 311d

Into a 250 mL 3-necked round-bottom were added 311c (6 g, 20,740 mmol, 1 equiv), Lawesson Reagent (16.78 g, 41.480 mmol, 2 equiv) and THF (60 mL) at room temperature. The resulting mixture was stirred for overnight at 40° C. The reaction was quenched with sat. NH4Cl (aq.) at room temperature. The aqueous layer was extracted with EtOAc (3×100 mL), and the resulting mixture was concentrated under reduced pressure. The residue was purified by silica gel column chromatography, eluted with PE/EA (2:1) to afford 311d (4 g, 62.42%) as a yellow oil.

### 5. Synthesis of 311e

To a solution of 311d (1.3 g, 4.524 mmol, 1 equiv) in 25 mL MeOH was added Pd/C (10%, 0.13 g) under nitrogen atmosphere in a 250 mL round-bottom flask. The mixture was hydrogenated at room temperature for 5 h under hydrogen atmosphere using a hydrogen balloon, filtered through a Celite pad and the mixture was concentrated under reduced pressure. This resulted in 311e (1.1 g, 89.75%) as a yellow oil.

### 6. Synthesis of 311f

Into a 40 mL vial were added 311e (300 mg, 1,166 mmol, 1 equiv), 1-2 (400.48 ng, 1.399 mmol, 1.2 equiv), STAB (741.16 mg, 3.498 mmol, 3 equiv) and DCE (5 mL) at room temperature. The mixture was stirred for overnight at room temperature. The reaction was quenched with sat. NH4Cl (aq.) (20 mL) at room temperature. The aqueous layer was extracted with CH2Cl2 (3×20 mL) and the mixture was concentrated under reduced pressure. The residue was purified by Prep-TLC (CH2Cl2/MeOH 20:1) to afford 311f (450 mg, 69.50%) as a yellow solid.

### 7. Synthesis of 311

Into a 100 mL round-bottom flask were added 311f (400 mg, 0.720 mmol, 1 equiv), DCM (12 mL) and pyridine (348.77 mg, 4.320 mmol, 6 equiv) at room temperature. To the above mixture was added triphosgene (109.03 mg, 0,360 mmol, 0.5 equiv) at 0° C. The resulting mixture was stirred for 5 min at 0° C. The reaction was quenched with sat. NaHCO₃ (aq.) (30 mL) at room temperature. The aqueous layer was extracted with CH2Cl2 (3×15 mL). The resulting mixture was concentrated under vacuum. The residue was purified by reverse flash chromatography with the following conditions: column, 120 g C18 silica gel; mobile phase, 0.1% NH4HCO3 in water, 50% to 60% gradient in 10 min; detector, UV 254 nm. This resulted in 311 (101.2 mg, 25.28%) as a yellow solid.

LC-MS: (ES, m/z): [M+H]+ 554. H-NMR: (400 MHz, CDCL3, δ ppm): 0.45-0.55 (m, 2H), 0.56-0.68 (m, 2H), 0.75-1.02 (d, 4H), 1.53-1.75 (d, 5H), 1.85-2.06 (m, 1H), 2.70-3.02 (s, 2H), 3.11-3.21 (m, 4H), 3.23-3.46 (s, 2H), 6.75-6.87 (s, 1H), 6.99-7.14 (d, 1H), 7.34-7.42 (m, 1H), 7.45-7.55 (d, 1H), 7.55-7.61 (d, 1H), 7.61-7.67 (s, 1H), 7.69-7.85 (s, 1H), 9.02-9.11 (s, 1H).

### Example 312. Synthesis of Compound 312

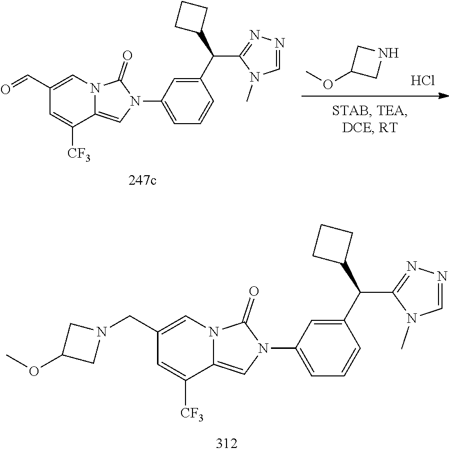

### Synthesis of 312

To a stirred solution of 247c (300 mg, 0.659 mmol, 1.00 equiv) and 3-methoxyazetidine hydrochloride (162.80 mg, 1.318 mmol, 2 equiv) in DCE (5 mL) were added TEA (133.31 mg, 1.318 mmol, 2 equiv) at room temperature. The mixture was stirred for 30 min at room temperature. To the above mixture was added STAB (279.21 mg, 1.318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×20 mL). The resulting mixture was concentrated under reduced pressure. The crude product (160 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 24% B to 54% B in 8 min, Wave Length: 220 nm; RT1 (min): 7.02) to afford 312 (136.4 mg, 38.93%) as a yellow solid.

LC-MS: (ES, m/z): [M+H]+: 527. H-NMR: (400 MHz, DMSO-d6, ppm): δ 1.62-1.88 (m, 5H), δ 2.08 (s, 1H), δ 2.85-2.87 (d, 2H), δ 3.15 (s, 3H), δ 3.21-3.23 (d, 1H), δ 3.32-3.34 (d, 2H), δ 3.45 (s, 3H), δ 3.47-3.49 (d, 2H), δ 3.99-4.01 (d, 1H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 2H), δ 7.75 (s, 1H), δ 8.35 (s, 1H).

### Example 313. Synthesis of Compound 313

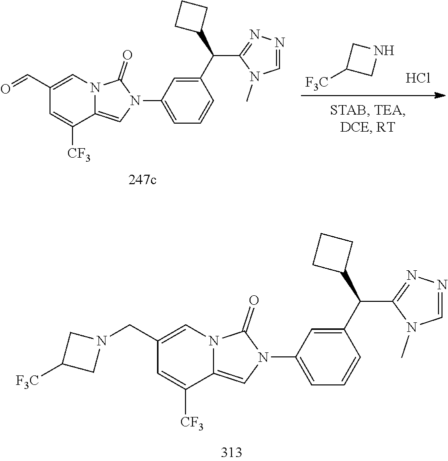 [SMILES: Cn1cnnc1[C@@H](c1cccc(-n2cc3c(C(F)(F)F)cc(C=O)cn3c2=O)c1)C1CCC1 ; FC(F)(F)C1CNC1 ; Cl ; Cn1cnnc1[C@@H](c1cccc(-n2cc3c(C(F)(F)F)cc(CN4CC(C(F)(F)F)C4)cn3c2=O)c1)C1CCC1]

### Synthesis of 313

To a stirred solution of 247c (300 mg, 0.659 mmol, 1.00 equiv) and 3-(trifluoromethyl)azetidine hydrochloride (212.83 mg, 1.318 mmol, 2 equiv) in DCE (5 mL) were added TEA (133.31 mg, 1.318 mmol, 2 equiv) at room temperature. The mixture was stirred for 30 min at room temperature. To the above mixture was added STAB (279.21 ng, 1.318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×20 mL). The resulting mixture was concentrated under reduced pressure. The crude product (143 mg) was purified by Prep-PLC with the following conditions (Column: XBridge Shield RP18 OBD Column, 30*150 mm, 5 μm; Mobile Phase A: Water (10 mmol % L NH4HCO3+0.1% NH3·H2O), Mobile Phase B: ACN; Flow rate: 60 mL/min; Gradient: 36% B to 60% B in 8 min, 60% B; Wave Length: 220 nm; RT1 (min): 7.28) to afford 313 (116.0 mg, 31.07%) as a yellow solid,

LC-MS: (ES, m/z): [M+H]+: 565. H-NMR: (400 MHz, DMSO-d6, ppm): δ 1.62-1.88 (m, 5H), δ 2.08 (s, 1H), δ 3.21-3.26 (m, 3H), δ 3.33-3.37 (m, 8H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 3H), δ 8.32 (s, 1H).

### Example 314. Synthesis of Compound 314

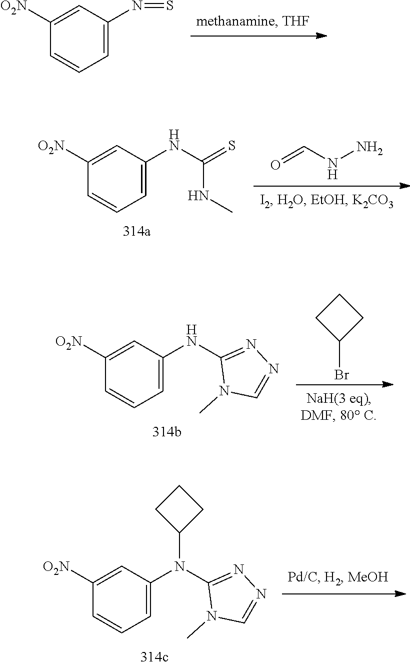

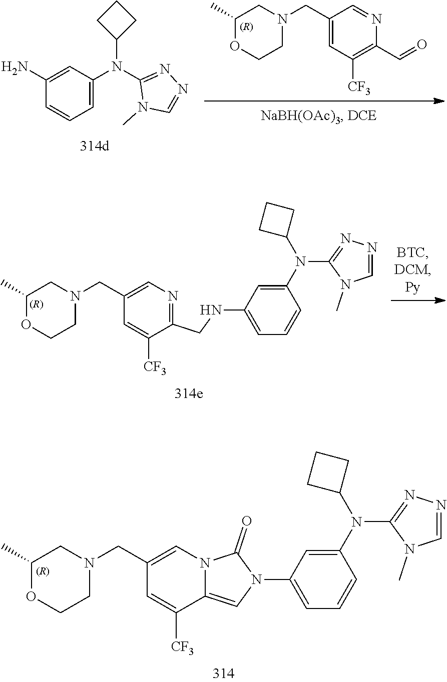

### 1. Synthesis of 314-1

Into a 500 mL 3-necked round-bottom flask were added 1-isothiocyanato-3-nitrobenzene (25 g, 138.750 mmol, 1 equiv), Methylamine 2M in THE (138.77 mL, 277.545 mmol, 2.00 equiv) and THF (250 mL) at room temperature. The resulting mixture was stirred for overnight at room temperature. The resulting mixture was concentrated under vacuum. The residue was purified by silica gel column chromatography, eluted with DCM/MeOH (60:1) to afford 314-1 (26 g, 86.93%) as a yellow

### 2. Synthesis of 314-2

Into a 500 mL 3-necked round-bottom flask were added 314-1 (26 g, 123083 mmol, 1 equiv), N-formylhydrazine (14.78 g, 246,166 mmol, 2 equiv), K2CO3 (42.53 g, 307.707 mmol, 2.5 equiv), EtOH (260 mL) and 1-120 (50 mL) at room temperature. To the above mixture was added 12 (37.49 g, 147.700 mmol, 1.2 equiv) at room temperature. The resulting mixture was stirred for overnight at room temperature. The resulting mixture was diluted with water (500 mL). The aqueous layer was extracted with DCM (3×200 mL). The resulting mixture was concentrated under vacuum. The residue was purified by silica gel column chromatography, eluted with DCM/MeOH (80:1) to afford 314-2 (8 g, 28.17%) as a yellow solid.

### 3. Synthesis of 314-3

To a stirred solution of 314-2 (2 g, 9.124 mmol, 1 equiv) and DMF (30 mL) was added NaH (L 0.09 g, 27.372 mmol, 3 equiv, 60%) at room temperature. The mixture was stirred for 1 h at room temperature. To the above mixture was added bromocyclobutane (3.70 g, 27.372 mmol, 3 equiv) dropwise at room temperature. The resulting mixture was stirred for overnight at 80° C. The reaction was quenched with sat. NH4Cl (aq.) (100 mL) at room temperature. The aqueous layer was extracted with EtOAc (3×40 mL). The resulting mixture was concentrated under vacuum. The residue was purified by Prep-TLC (DCM/MeOH=40:1) to afford 314-3 (600 mg, 23.10%) as a brown oil.

### 4. Synthesis of 314-4

To a solution of 314-3 (600 mg, 2.195 mmol, 1 equiv) in 10 mL MeOH was added Pd/C (20%, 120 mg) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure. The residue was purified by Prep-TLC (DCM/MeOH=20:1) to afford 314-4 (500 mg, 87.05%) as a colorless oil.

### 5. Synthesis of 314-5

Into a 25 mL round-bottom flask were added 314-4 (300 mg, 1.233 mmol, 1 equiv), PH-I-2 (426.52 mg, 1.480 mmol, 1.2 equiv), STAR (522.63 mg, 2.466 mmol, 2 equiv) and DCE (5 mL) at room temperature. The resulting mixture was stirred for overnight at room temperature. The reaction was quenched with sat. NH4Cl (aq.) (50 mL) at room temperature. The aqueous layer was extracted with EtOAc (3×20 mL). The resulting mixture was concentrated under vacuum. The residue was purified by Prep-TLC (DCM/MeOH=30:1) to afford 314-5 (200 mg 29.89%) as a brown oil.

### 6. Synthesis of 314-0

Into a 50 mL round-bottom flask were added 314-5 (180 mg, 0.349 mmol, 1 equiv), DCM (10 mL) and pyridine (220.92 mg, 2.792 mmol, 8 equiv) at room temperature. To the above mixture was added Triphosgene (72.52 mg, 0.244 mmol, 0.7 equiv) at 0° C. The resulting mixture was stirred for 5 min at 0° C. The reaction was quenched with sat. NaHCO3 (aq.) (30 mL) at room temperature. The aqueous layer was extracted with EtOAc (3×10 mL). The resulting mixture was concentrated under vacuum. The residue was purified by reverse flash chromatography with the following conditions: column, 60 g C18 silica gel; mobile phase, 0.1% NH4HCO3 in water, 40% to 60% gradient in 20 min; detector, UV 254 nm.

H-NMR-324 1H NMR (400 MHz, CDCl3) δ 1.16-1.18 (m, 3H), δ 1.74-1.78 (m, 2H), δ 1.80-1.87 (m, 1H), δ 2.02-2.07 (m, 2H), δ 2.12-2.21 (m, 1H), δ 2.34-2.42 (m, 2H), δ 2.72-2.79 (m, 2H), δ 3.26-3.33 (m, 2H), δ 3.43 (s, 3H), δ 3.62-3.70 (m, 2H), δ 3.87-3.91 (m, 1H), δ 4.48-4.52 (m, 1H), δ 6.58-6.61 (d, 1H), δ 6.77 (s, 1H), δ 6.96-7.02 (m, 1H), δ 7.09-7.11 (d, 1H), δ 7.20 (s, 1H), δ 7.32-7.36 (m, 1H), δ 7.63 (s, 1H), δ 8.21 (s, 1H).

### Example 315. Synthesis of Compound 315

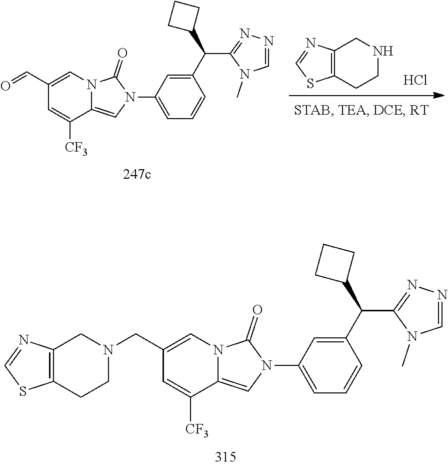

### Synthesis of 315

To a stirred solution of 247c (300 mg, 0.659 mmol, 1 equiv) and 4H,5H,6H,7H-[1,3]thiazolo[4,5-c]pyridine hydrochloride (232.73 mg, 1.318 mmol, 2 equiv) in DCE (5 ml) was added TEA (133.31 mg, 1.318 mmol, 2 equiv) at room temperature. The resulting mixture was stirred for 1h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×30 mL). The resulting mixture was concentrated under vacuum. The residue was purified by Prep-TLC (DCM/MeOH 18:1) to afford the crude product. The crude product was purified by Prep-HPLC with the following conditions (Column: XBridge Shield RP18 OBD Column, 30*150 mm, 5 μm; Mobile Phase A: Water (10 mmol/L NH4HCO3+0.1% NH3·H2O), Mobile Phase B: ACN; Flow rate: 60 mL/min; Gradient: 34% B to 52% B in 8 min, Wave Length: 220 nm; RT1 (min): 7.55) to afford 315 (50.6 mg, 13.21%) as a yellow solid.

LC-MS: (ES, m/z): [M+H]⁺ 580. H-NMR: (400 MHz, DMSO-d6, δ ppm): 1.63-1.83 (m, 5H), 2.07-2.12 (m, 1H), 2.81-2.91 (m, 4H), 3.19-3.25 (m, 1H), 3.43 (s, 3H), 3.54 (s, 2H), 3.66 (s, 2H), 4.25-4.28 (d, 1H), 7.07 (s, 1H), 7.18-7.20 (d, 1H), 7.33 (s, 1H), 7.69-7.77 (m, 3H), 8.33 (s, 1H), 8.90 (s, 1H).

### Example 316. Synthesis of Compound 316

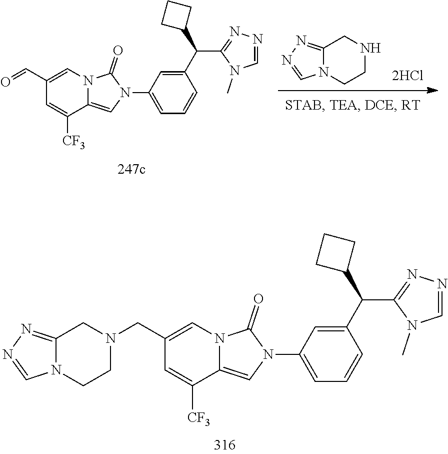

### Synthesis of 316

To a stirred solution of 247c (300 mg, 0.659 mmol, 1 equiv) and 5H,6H,7H,8H-[1,2,4]triazolo[4,3-a]pyrazine hydrochloride (317.38 ng, 1.977 mmol, 3 equiv) in DCE (5 mL) was added TEA (133.31 mg, 1.318 mmol, 2 equiv) at room temperature. The resulting mixture was stirred for 1h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-HPLC with the following conditions (Column: XSelect CSH Fluoro Phenyl, 30*150 mm, 5 μm; Mobile Phase A: Water (10 mmol/L NH4HCO3), Mobile Phase B: ACN; Flow rate: 60 mL/min; Gradient: 30% B to 50% B in 7 min, 50% B; Wave Length: 220 nm; RT1 (min): 6.25) to afford 316 (34.6 mg, 9.23%) as a yellow solid.

LC-MS: (ES, m/z): [M+H]+ 564. H-NMR: (400 MHz, DMSO-d6, δ ppm): 1.63-1.93 (m, 5H), 2.07-2.12 (m, 1H), 2.89-2.92 (m, 2H), 3.19-3.23 (m, 1H), 3.43 (s, 3H), 3.56 (s, 2H), 3.80 (s, 2H), 4.03-4.06 (m, 2H), 4.25-4.28 (d, 1H), 7.07 (s, 1H), 7.19-7.21 (d, 1H), 7.33 (s, 1H), 7.43-7.45 (m, 1H), 7.70-7.74 (m, 2H), 7.80 (s, 1H), 8.33 (s, 1H), 8.43 (s, 1H).

### Example 317. Synthesis of Compound 317

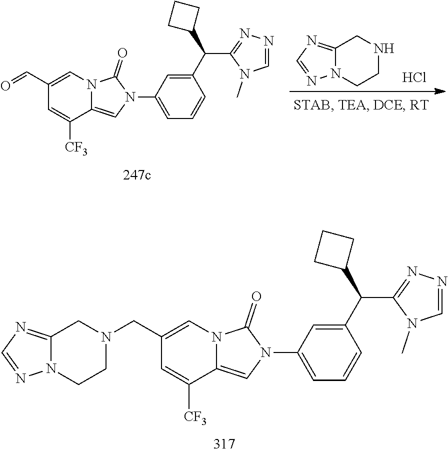

### Synthesis of 317

To a stirred solution of 247c (300 mg, 0,659 mmol, 1 equiv) and 5H,6H,7H,8H-[1,2,4]triazolo[1,5-a]pyrazine hydrochloride (528.97 mg, 3.295 mmol, 5 equiv) in DCE (5 mL) was added TEA (333.28 mg, 3.295 mmol, 5 equiv) at room temperature. The resulting mixture was stirred for 1h at room temperature. To the above mixture was added STAB (418.82 mg, 1.977 mmol, 3 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-HPLC with the following conditions (Column: Kinetex EVO prep C18, 30*150, 5 μm; Mobile Phase A: Water (10 mmol/L NH4HCO3), Mobile Phase B: ACN; Flow rate: 60 mL/min; Gradient: 15% B to 42% B in 7 min, Wave Length: 220 nm; RT1 (min): 9.63) to afford 317 (20.0 mg, 5.28%) as a yellow solid.

LC-MS: (ES, m/z): [M+H]+ 564. H-NMR: (400 MHz, DMSO-d6, δ ppm): 1.63-1.93 (m, 5H), 2.07-2.12 (m, 1H), 3.00-3.08 (m, 2H), 3.19-3.23 (m, 1H), 3.43 (s, 3H), 3.56 (s, 2H), 3.79 (s, 2H), 4.12-4.14 (m, 2H), 4.25-4.28 (d, 1H), 7.08 (s, 1H), 7.19-7.21 (d, 1H), 7.33 (s, 1H), 7.43-7.46 (m, 1H), 7.69-7.73 (m, 2H), 7.81 (s, 1H), 7.91 (s, 1H), 8.33 (s, 1H).

### Example 318. Synthesis of Compound 318

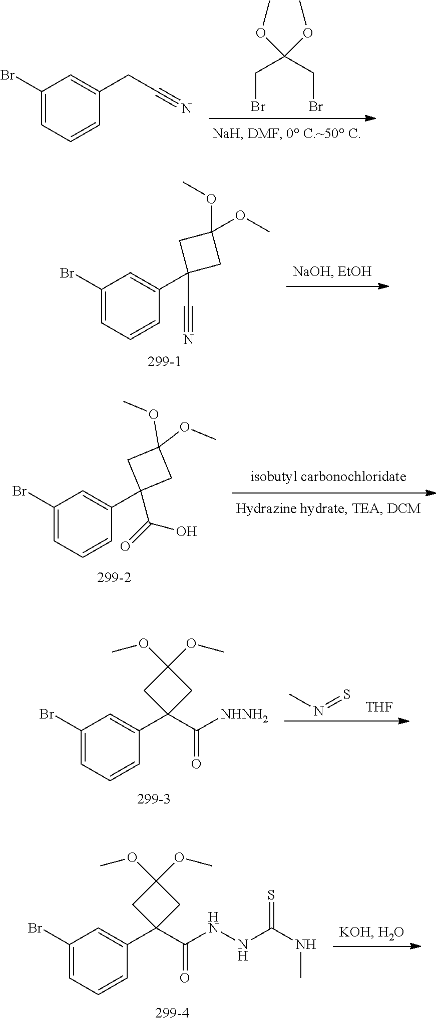

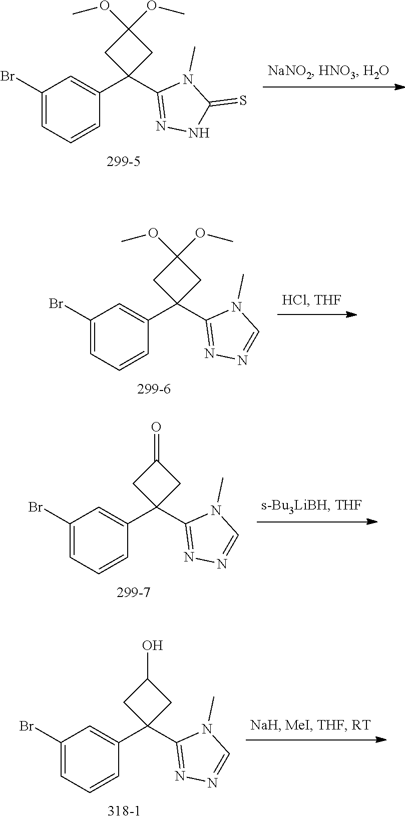 [SMILES: COC1(OC)CC(c2cccc(Br)c2)(c2n[nH]c(=S)n2C)C1 ; COC1(OC)CC(c2cccc(Br)c2)(c2nncn2C)C1 ; Cn1cnnc1C1(c2cccc(Br)c2)CC(=O)C1 ; Cn1cnnc1C1(c2cccc(Br)c2)CC(O)C1]

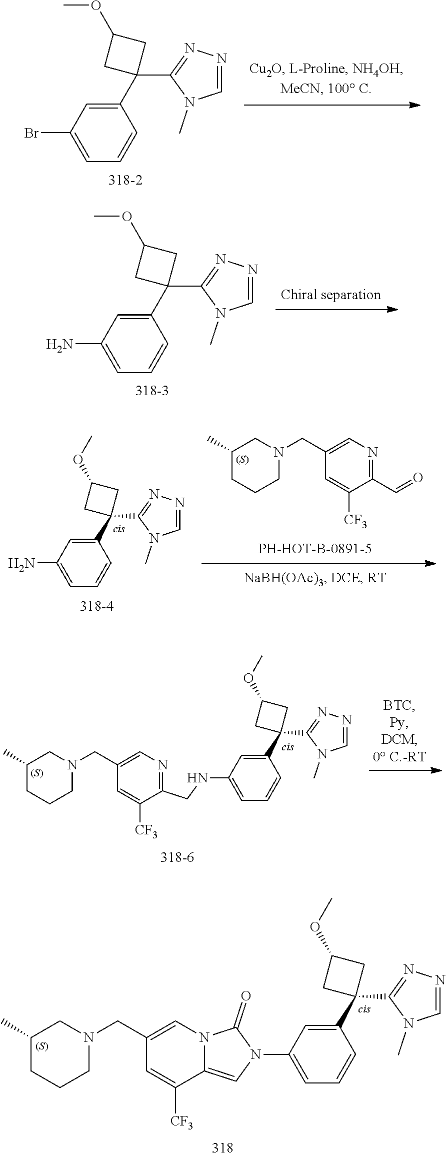

### 1. Synthesis of 279-1

To a stirred solution of NaH (24.48 g, 1020.164 mmol, 2 equiv) in DMF (1000 mL) was added 2-(3-bromophenyl)acetonitrile (100 g, 510.082 mmol, 1 equiv) dropwise at 0° C. under nitrogen atmosphere, followed by 1,3-dibromo-2,2-dimethoxypropane (133.61 g, 510.082 mmol, 1 equiv) at 60° C. under nitrogen atmosphere. The resulting mixture was stirred overnight at 60° C. under nitrogen atmosphere. The reaction was quenched with NH4Cl (aq.) (2 L) at room temperature. The aqueous layer was extracted with EtOAc (2×1000 mL). The resulting mixture was concentrated under reduced pressure. The residue was purified by silica gel column chromatography, eluted with PE/EA (10:1) to afford 279-1 (60 g, 39.72%) as off-white solid.

### 2. Synthesis of 279-2

To a stirred solution of 279-1 (60 g, 202.590 mmol, 1 equiv) in EtOH (600 mL) was added NaOH (28.36 g, 709.065 mmol, 3.5 equiv) in H2O (600 mL) at room temperature. The resulting mixture was stirred overnight at 80° C. The EtOH was concentrated under vacuum. The mixture was acidified to pH 2 with HCl (1M). The aqueous layer was extracted with EtOAc (4×300 mL). The combined organic layers were dried over anhydrous Na2SO4. After filtration, the filtrate was concentrated under reduced pressure. This resulted in 279-2 (43 g, 67.35%) as off-white solid.

### 3. Synthesis of 279-3

To a stirred solution of 279-2 (43 g, 136.437 mmol, 1 equiv) and TEA (27.61 g, 272.874 mmol, 2 equiv) in DCM (1500 mL) was added isobutyl carbonochloridate (27.95 g, 204.656 mmol, 1.5 equiv) dropwise at 0° C. under nitrogen atmosphere. The resulting mixture was stirred for 30 min at 0° C. To the above mixture was added hydrazine hydrate (27.32 g, 545.748 mmol, 4 equiv) dropwise at −30° C. The resulting mixture was stirred for additional 40 min at room temperature. The reaction was quenched with water (2 L) at room temperature. The aqueous layer was extracted with CH2Cl2 (3×800 mL). The combined organic layers were dried over anhydrous Na2SO4. After filtration, the filtrate was concentrated under reduced pressure. This resulted in 279-3 (50 g, 83.49%) as off-white solid. The crude product was used in the next step directly without further purification.

### 4. Synthesis of 279-4

To a stirred solution of 318-3 (50 g, 113.915 mmol, 1 equiv) in tetrahydrofuran (500 mL) was added methyl isothiocyanate (24.98 g, 341,745 mmol, 3 equiv) at room temperature. The resulting mixture was stirred for overnight at room temperature. The resulting mixture was diluted with water (400 mL). The resulting mixture was concentrated under vacuum. The precipitated solids were collected by filtration and washed with water (200 mL). This resulted in 279-4 (65 g, 99.28%) as off-white solid. The crude product was used in the next step directly without further purification.

### 5. Synthesis of 279-5

To a stirred solution of KOH (72.52 g, 1292.536 mmol, 8 equiv) in 1120 (1.2 L) was added 318-4 (65 g, 161.567 mmol, 1 equiv) at room temperature. The resulting mixture was stirred overnight at room temperature. The residue was neutralized to pH 7 with HCl (aq.) (1M). The precipitated solids were collected by filtration and washed with water (200 mL). This resulted in 279-5 (45 g, 72.48%) as off-white solid.

### 6. Synthesis of 279-6

To a stirred mixture of 318-5 (45 g, 117.099 mmol, 1 equiv) and NaNO2 (80.79 g, 1170.990 mmol, 10.00 equiv) in EA (250 mL)/1120 (250 ml) was added HNO3 (1170 mL, 1170.990 mmol, 10.00 equiv, 1M) dropwise at room temperature. The resulting mixture was stirred overnight at room temperature. The mixture was neutralized to pH 7 with saturated Na2CO3 (aq.). The aqueous layer was extracted with EtOAc (5×500 L). The resulting mixture was concentrated under vacuum. This resulted in 279-6 (35 g, 65.34%) as off-white solid.

### 7. Synthesis of 279-7

To a stirred solution of 279-6 (35 g, 78,747 mmol, 1 equiv, 77%) in THF (200 mL) was added HCl (150 mL, 4M) at room temperature. The resulting mixture was stirred overnight at 80° C. The mixture was neutralized to pH 8 with NaHCO₃-(aq.). The aqueous layer was extracted with EtOAc (3×100 mL). The resulting mixture was concentrated under vacuum. The residue was purified by reverse flash chromatography with the following conditions: column, C18 silica gel; mobile phase, MeCN in water, 0% to 40% gradient in 20 min; detector, UV 220 nm. This resulted in 279-7 (18 g, 74.66%) as a white solid.

### 8. Synthesis of 318-1

To a stirred solution of 279-7 (34.5 g, 112,685 mmol, 1 equiv) in THF (400 mL) was added lithium triisobutylhydroborate (42.84 g, 225.370 mmol, 2 equiv) dropwise at 0′C. The resulting mixture was stirred for 4 h at room temperature. The reaction was quenched with NH4Cl (aq.) (500 mL) at room temperature. The aqueous layer was extracted with EtOAc (5×400 ml). The resulting mixture was concentrated under vacuum. The residue was purified by reverse flash chromatography with the following conditions: column, C18 silica gel; mobile phase, MeCN in Water (0.1% FA), 10% to 60% gradient in 30 min; detector, UV 220 nm. This resulted in 318-1 (17 g, 48.95%) as a white solid,

### 9. Synthesis of 318-2

To a stirred mixture of 318-1 (17 g, 55.163 mmol, 1 equiv) in THF (200 mL) was added NaH (2.65 g, 110.326 mmol, 2 equiv) at 0° C. under nitrogen atmosphere. The resulting mixture was stirred for 1 h at 0° C. under nitrogen atmosphere. To the above mixture was added MeI (9.40 g, 66.196 mmol, 1.2 equiv) at 0° C. The resulting mixture was stirred overnight at room temperature. The reaction was quenched with NH4Cl (aq.) (500 mL) at room temperature. The aqueous layer was extracted with EtOAc (4×300 mL). The resulting mixture was concentrated under vacuum. The residue was purified by reverse flash chromatography with the following conditions: column, C18 silica gel; mobile phase, MeCN in Water (0.1% FA), 10% to 60% gradient in 30 min; detector, UV 220 nm. This resulted in 318-2 as a white solid.

### 10. Synthesis of 318-10

To a stirred mixture of 318-2 (11 g, 34.140 mmol, 1 equiv) and Cu2O (4.89 g, 34.140 mmol, 1 equiv) in NH40H (500 mL) and MeCN (500 mL) was added L-Proline (0.83 g, 3.414 mmol, 0.1 equiv) at room temperature. The resulting mixture was stirred overnight at 100° C. The mixture was allowed to cool down to room temperature. The resulting mixture was filtered; the filter cake was washed with MeOH (3×100 mL). The resulting mixture was concentrated under vacuum. The residue was purified by reverse flash chromatography with the following conditions: column, C18 silica gel; mobile phase, MeCN in Water (0.1% FA), 10% to 50% gradient in 30 min; detector, UV 254 nm. This resulted in 318-3 (6.2 g, 70.30%) as off-white solid.

### 11. Synthesis of 318-1

The 318-3 (6.2 g) was purified by Prep-SFC with the following conditions (Column: Lux 5 um Cellulose-3, 5*25 cm, 5 man; Mobile Phase A: CO2. Mobile Phase B: MeOH (0.1% 2M NH3-MEOH); Flow rate: 150 mL/min; Gradient: isocratic 20% B; Column Temperature (° C.): 35; Back Pressure (bar): 100; Wave Length: 220 am; RT1 (min): 4.78; RT2 (min): 5.94; the first peak was product) to afford 318-1 (2.7 g) as an off-white solid.

### 12. Synthesis of 318-6

A solution of 318-1 (2.6 g 10.065 mmol, 1 equiv) and 1-2 (3.17 g, 11.072 mmol, 1.1 equiv) in DCE (50 mL) was stirred overnight at room temperature. To the above mixture was added NaBH(OAc)3 (4.27 g, 20.130 mmol, 2 equiv) at room temperature. The resulting mixture was stirred for additional 4 h at room temperature. The reaction was quenched with NaHCO₃ (aq.) (150 mL) at room temperature. The aqueous layer was extracted with CH2Cl2 (5×100 mL). The residue was purified by silica gel column chromatography, eluted with CH2Cl2/MeOH (50:1) to afford 318-6 (2.8 g, 52.63%) as off-white solid,

### 13. Synthesis of 318-0

To a stirred mixture of 318-6 (2.8 g, 5.303 mmol, 1 equiv) and Pyridine (5.39 g, 31.818 mmol, 6 equiv) in DCM (50 mL) was added triphosgene (0.596 g 2.015 mmol, 0.38 equiv) at 0° C. The resulting mixture was stirred for 30 min at room temperature. The reaction was quenched with NaHCO₃ (aq.) (100 mL) at room temperature. The aqueous layer was extracted with CH2Cl2 (4×50 mL). The resulting mixture was concentrated under reduced pressure. The residue was purified by reverse flash chromatography with the following conditions: column, C18 silica gel; mobile phase, MeCN in Water (10 mmol/L NH4HCO3), 0% to 60% gradient in 40 min; detector, UV 254 nm. This resulted in 318 (1,577 g, 54.38%) as a yellow solid.

LC-MS-318: (ES, m/z): [M+H]+ 555

H-NMR-318: (400 MHz, DMSO-d6, δ ppm): 0.80-0.91 (m, 4H), 1.41-1.53 (m, 1H), 1.55-1.67 (m, 4H), 1.87-1.92 (m, 1H), 2.69-2.83 (m, 4H), 3.11-3.17 (m, 2H), 3.19 (s, 3H), 3.24 (s, 5H), 4.04-4.12 (m, 1H), 7.01 (s, 1H), 7.25-7.27 (m, 1H), 7.41 (s, 1H), 7.49-7.53 (m, 1H), 7.67-7.69 (m, 2H), 7.79 (s, 1H), 8.32 (s, 1H).

### Example 319. Synthesis of Compound 319

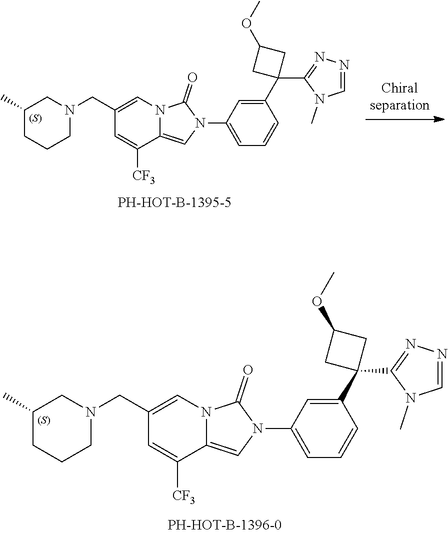

### Synthesis of 319

318-5 (280 ng) was purified by chiral separation with the following conditions (Column: CHIRALPAK 1, 2*25 cm, 5 μm; Mobile Phase A: Hex (0.5% 21 NH3-MeOH). Mobile Phase B: EtOH:DCM=1:1; Flow rate: 20 mL/min; Gradient: 35% B to 35% B in 20 min; Wave Length: 220/254 nm; RT1 (min): 6.20; RT2 (min): 12.59; the first peak is product) to afford 319 (155.2 mg, 68.57%) as a yellow solid.

LC-MS-319 (ES, m/z): [M+H]+ 555. H-NMR-319 (400 MHz, DMSO-d6, δ ppm): 0.80-0.91 (m, 4H), 1.40-1.48 (m, 1H), 1.50-1.78 (m, 4H), 1.86-1.91 (m, 1H), 2.54-2.55 (m, 2H), 2.72-2.77 (m, 2H), 3.16 (s, 2H), 3.18 (s, 5H), 3.30-3.31 (m, 1H), 3.34-3.35 (m, 1H), 3.82-3.89 (m, 1H), 7.00 (s, 1H), 7.07-7.09 (d, 1H), 7.40 (s, 1H), 7.46-7.50 (t, 1H), 7.64-7.70 (m, 2H), 7.71 (s, 1H), 8.39 (s, 1H).

### Example 320. Synthesis of Compound 320

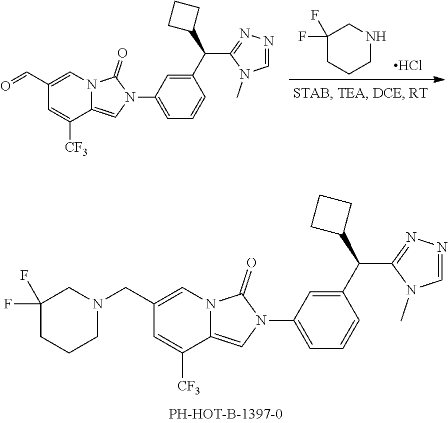

### Synthesis of 320-0

To a stirred solution of 247c (300 mg, 0.659 mmol, 1.00 equiv) and 3,3-difluoropiperidine hydrochloride (207.61 mg, 1.318 mmol, 2 equiv) in DCE (5 mL) was added TEA (133.31 mg, 1.318 mmol, 2 equiv) at room temperature. The resulting mixture was stirred for 1 h at room temperature. To the above mixture was added STAB (279.21 mg, 1,318 mmol, 2 equiv) at room temperature. The resulting mixture was stirred for additional overnight at room temperature. The reaction was quenched with sat. NH4Cl (aq.) at room temperature. The aqueous layer was extracted with CH2Cl2 (2×20 mL). The resulting mixture was concentrated under vacuum. The crude product (143 mg) was purified by Prep-HPLC with the following conditions (Column: XBridge Prep OBD C1 8 Column, 30*150 mm, 5 μm; Mobile Phase A: Water (10 mmol/L NH4HCO3), Mobile Phase B: ACN; Flow rate: 60 mL/min; Gradient: 37% B to 67% B in 8 min, 67% B; Wave Length: 220 nm; RT1 (min): 7.37) to afford 320 (126.0 mg, 36.30%) as a yellow solid.

LC-MS-320 (ES, m/z): [M+H]+: 561

H-NMR-320 (400 MHz, DMSO-d6, ppm): δ 1.62-1.70 (m, 3H), δ 1.71-1.83 (m, 5H), δ 1.84-1.95 (m, 2H), δ 2.11 (s, 1H), δ 2.51-2.53 (d, 2H), δ 2.65-2.67 (m, 2H), δ 3.12 (s, 1H), δ 3.46-3.49 (m, 5H), δ 4.25-4.27 (d, 1H), δ 7.30 (s, 1H), δ 7.41-7.45 (m, 1H), δ 7.64-7.68 (m, 1H), δ 7.73 (s, 1H), δ 7.65-7.68 (m, 3H), δ 8.32 (s, 1H).

### Example 321. Synthesis of Compound 321

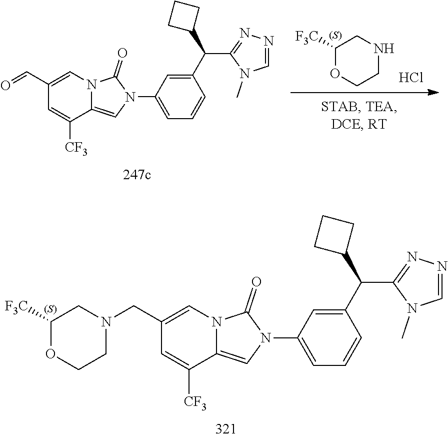

### Synthesis of 321

To a stirred solution of 247c (300 mg, 0.659 mmol, 1.00 equiv) and (2S)-2-(trifluoromethyl)morpholine hydrochloride (252.39 mg, 1.318 mmol, 2 equiv) in DCE (5 mL) were added TEA (133.31 mg, 1.318 mmol, 2 equiv) at room temperature. The mixture was stirred for 30 min at room temperature. To the above mixture was added STAB (279.21 mg, 1.318 mmol, 2 equiv) at room temperature. The resulting mixture was stirred for overnight at room temperature. The reaction was quenched by the addition of sat. NH4Cl (aq.) (10 ml) at room temperature. The resulting mixture was extracted with CH2Cl2 (3×20 mL). The resulting mixture was concentrated under reduced pressure. The crude product (142 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 38% B to 65% B in 8 min, 65% B; Wave Length: 220 nm; RT1 (min): 7.53) to afford 321 (108.9 mg, 27.44%) as a yellow solid.

LC-MS-321: (ES, m/z): [M+H]+: 595. H-NMR-321: (400 MHz, DMSO-d6, ppm): δ 1.61-1.86 (m, 5H), δ 2.05-2.23 (m, 3H), δ 2.73-2.78 (m, 1H), δ 2.93-2.96 (m, 1H), δ 3.13-3.23 (m, 1H), δ 3.31-3.33 (d, 2H), δ 3.45 (s, 3H), δ 3.61-3.65 (m, 1H), δ 3.89-3.96 (m, 1H), δ 4.12-4.22 (m, 1H), δ 4.32-4.34 (d, 1H), δ 7.02 (s, 1H), δ 7.21-7.23 (d, 1H), δ 7.33 (s, 1H), δ 7.41-7.45 (m, 1H), δ 7.71-7.73 (d, 1H), δ 7.75 (s, 2H), δ 8.33 (s, 1H).

### Example 322. Synthesis of Compound 322

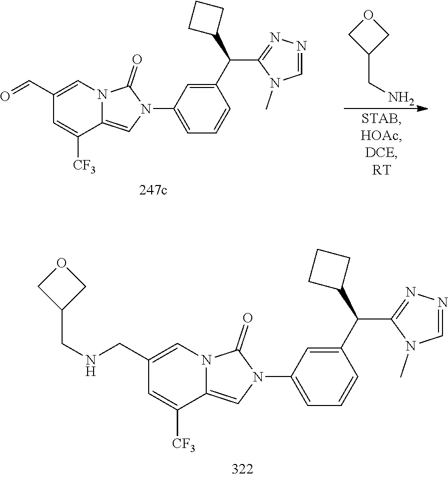 [SMILES: Cn1cnnc1[C@@H](c1cccc(-n2cc3c(C(F)(F)F)cc(C=O)cn3c2=O)c1)C1CCC1 ; NCC1COC1 ; Cn1cnnc1[C@@H](c1cccc(-n2cc3c(C(F)(F)F)cc(CNCC4COC4)cn3c2=O)c1)C1CCC1]

### Synthesis of 322-0

To a stirred solution of 247 C (300 mg, 0.659 mmol, 1 equiv) and 1-(oxetan-3-yl)methanamine (114.77 mg, 1.318 mmol, 2 equiv) in DCE (4 mL) were added STAB (279:21 mg, 1.318 mmol, 2 equiv) and HOAc (39.56 mg, 0.659 mmol, 1 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×20 mL). The resulting mixture was concentrated under reduced pressure. The crude product (101 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 19% B to 49% B in 8 min; Wave Length: 220 nm; RT1 (min): 7.57) to afford 322 (79.9 mg, 22.94%) as a yellow solid.

LC-MS-322 (ES, m/z): [M+H]+: 527. H-NMR-322 (400 MHz, DMSO-d6, ppm): δ 1.68-1.89 (m, 5H), δ 2.01-2.16 (m, 1H), δ 2.71-2.81 (m, 2H), δ 2.96-3.07 (m, 1H), δ 3.15-3.24 (m, 1H), δ 3.44 (s, 3H), δ 3.51-3.53-4.31 (d, 2H), δ 4.22-4.31 (m, 3H), δ 4.61-4.64 (m, 2H), δ 7.00 (s, 1H), δ 7.18-7.20 (d, 1H), δ 7.31 (s, 1H), δ 7.42-7.46 (m, 1H), δ 7.71-7.78 (m, 3H), δ 8.33 (s, 1H).

### Example 323. Synthesis of Compound 323

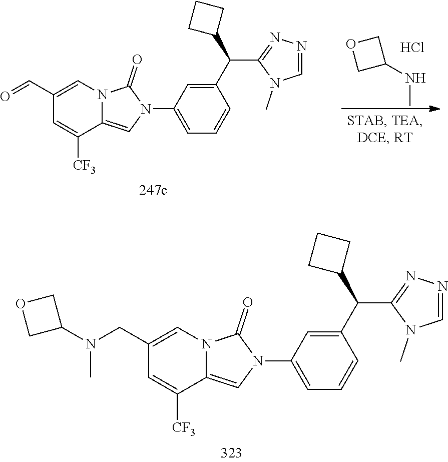

### Synthesis of 323

To a stirred solution of 247c (300 mg, 0,659 mmol, 1 equiv) and N-methyloxetan-3-amine hydrochloride (162.80 mg, 1.318 mmol, 2 equiv) in DCE (5 mL) was added TEA (133.31 mg, 1.318 mmol, 2 equiv) at room temperature. The resulting mixture was stirred for 1 h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The residue was purified by reverse flash chromatography with the following conditions: column, C18 silica gel; mobile phase, MeCN in water, 0% to 60% gradient in 15 min; detector, UV 254 nm. This resulted in 323 (97.3 mg, 27.94%) as a yellow solid.

LC-MS: (ES, m/z): [M+H]+ 527. H-NMR: (400 MHz, DMSO-d6, δ ppm): 1.63-1.93 (m, 5H), 2.02 (s, 3H), 2.07-2.12 (m, 1H), 3.16 (s, 2H), 3.19-3.25 (m, 1H), 3.43 (s, 3H), 3.60-3.64 (m, 1H), 4.25-4.28 (d, 1H), 4.46-4.4.49 (m, 2H), 4.53-4.4.56 (m, 2H), 7.04 (s, 1H), 7.18-7.20 (d, 1H), 7.33 (s, 1H), 7.42-7.46 (m, 1H), 7.69-7.74 (m, 3H), 8.33 (s, 1H).

### Example 324. Synthesis of Compound 324

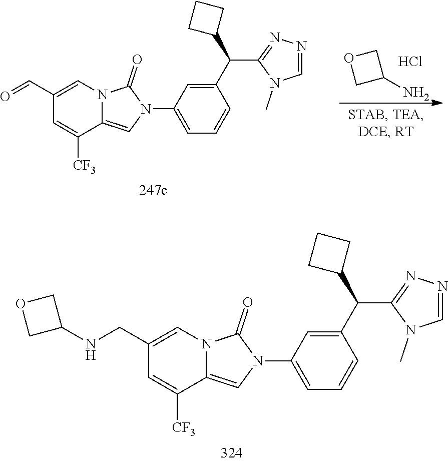 [SMILES: Cn1cnnc1[C@@H](c1cccc(-n2cc3c(C(F)(F)F)cc(C=O)cn3c2=O)c1)C1CCC1 ; NC1COC1 ; Cl ; Cn1cnnc1[C@@H](c1cccc(-n2cc3c(C(F)(F)F)cc(CNC4COC4)cn3c2=O)c1)C1CCC1]

### Synthesis of 324

To a stirred solution of 247c (300 mg, 0.659 mmol, 1 equiv) and oxetan-3-amine hydrochloride (144.32 mg, 1.318 mmol, 2 equiv) in DCE (5 mL) was added TEA (133.31 mg, 1.318 mmol, 2 equiv) at room temperature. The resulting mixture was stirred for 1 h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-TLC with the following conditions (Column: XBridge Shield RP18 OBD Column, 30*150 mm, 5 μm; Mobile Phase A: Water (10 mmol/L NH4HCO3+0.1% NH3.12O). Mobile Phase B: ACN; Flow rate: 60 mL/min; Gradient: 21% B to 46% B in 8 min, 46% B; Wave Length: 220 nm; RT1 (min): 7.37; Number Of Runs: 0) to afford 324 (62.6 mg, 18.47%) as a yellow solid.

LC-MS: (ES, m/z): [M+H]+ 513. H-NMR: (400 MHz, DMSO-d6, δ ppm): 1.63-1.93 (m, 5H), 2.07-2.12 (m, 1H), 2.98-3.11 (m, 1H), 3.19-3.25 (m, 1H), 3.43 (s, 3H), 3.45 (s, 2H), 3.85-3.89 (m, 1H), 4.25-4.32 (m, 2H), 4.57-4.61 (m, 2H), 7.12 (s, 1H), 7.18-7.20 (d, 1H), 7.31 (s, 1H), 7.42-7.46 (m, 1H), 7.68-7.74 (m, 3H), 8.33 (s, 1H).

### Example 325. Synthesis of Compound 325

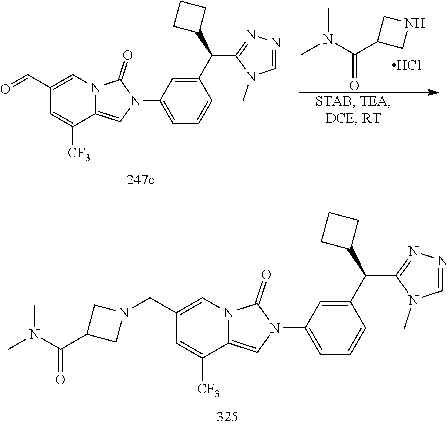

### 1. Synthesis of 325

To a stirred solution of 247c (300 mg, 0659 mmol, 1 equiv) and N,N-dimethylazetidine-3-carboxamide hydrochloride (216.88 mg, 1.318 mmol, 2 equiv) in DCE (5 mL) was added TEA (133.31 mg, 1.318 mmol, 2 equiv) at room temperature. The resulting mixture was stirred for 1 h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×30 mL). The resulting mixture was concentrated under vacuum. The residue was purified by Prep-TLC (DCM/MeOH 18:1) to afford the crude product. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 19% 3 to 49% B in 8 min, 49% B; Wave Length: 220 nm; RT1 (min): 7.60) to afford 325 (94.1 mg, 24.19%) as a yellow solid.

LC-MS: (ES, m/z): [M+H]+ 568. H-NMR: (400 MHz, DMSO-d6, δ ppm): 1.63-1.83 (m, 5H), 2.07-2.12 (m, 1H), 2.82 (s, 6H), 3.16-3.27 (m, 3H), 3.33 (s, 2H), 3.36-3.53 (m, 6H), 4.25-4.28 (d, 1H), 6.97 (s, 1H), 7.18-7.20 (d, 1H), 7.31 (s, 1H), 7.67-7.70 (m, 2H), 7.74 (s, 1H), 8.33 (s, 1H).

### Example 326. Synthesis of Compound 326

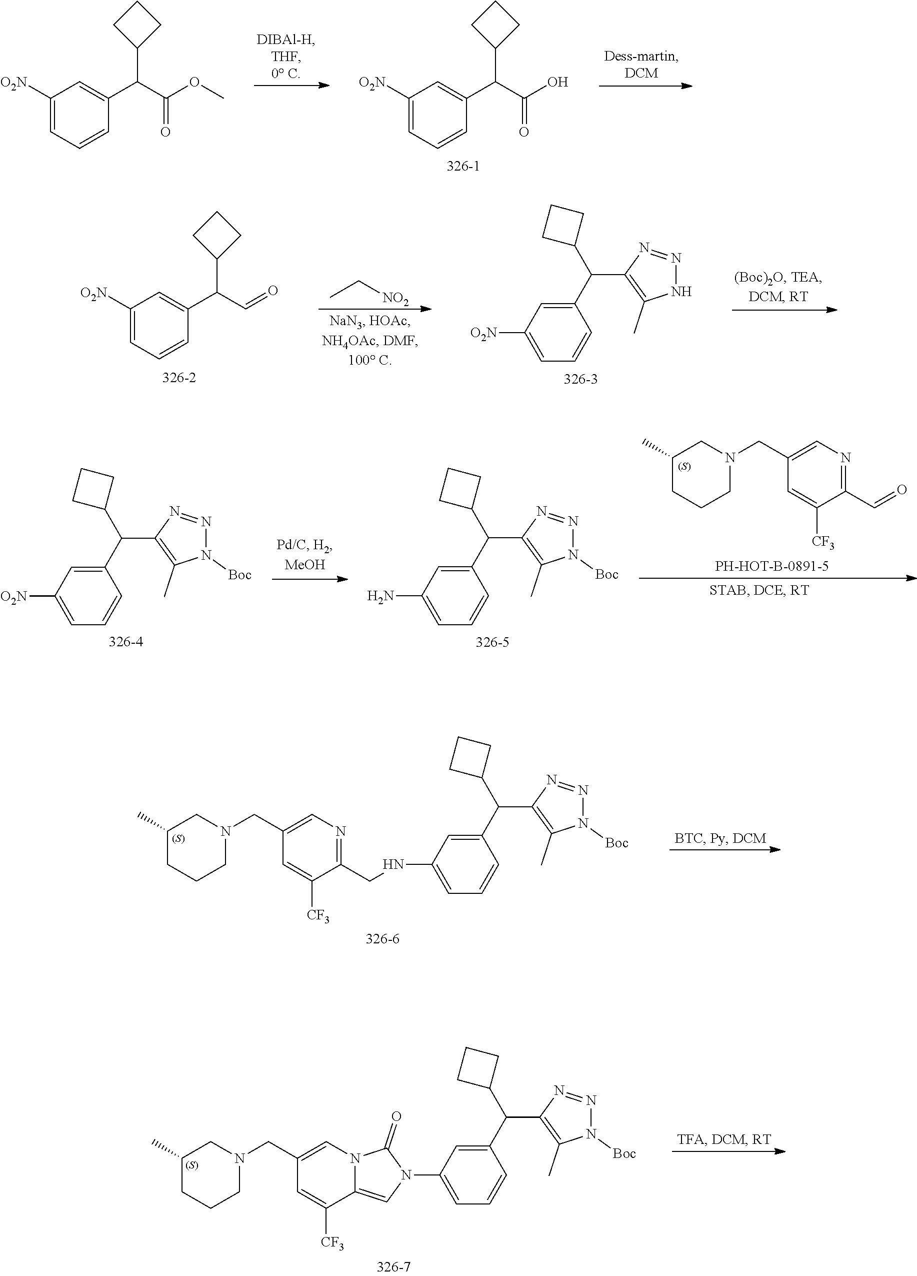

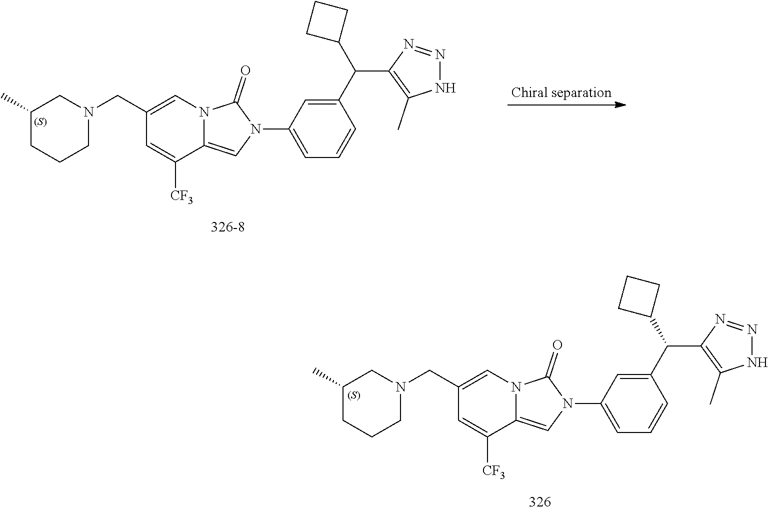

### 1. Synthesis of 326-1

To a stirred solution of methyl 2-cyclobutyl-2-(3-nitrophenyl)acetate (17 g, 68.200 mmol, 1 equiv) in THF (200 mL) was added DIBAl-H (24.25 g, 170.500 mmol, 2.5 equiv) dropwise at 0° C. under nitrogen atmosphere. The resulting mixture was stirred overnight at room temperature. The reaction was quenched with NH4Cl (aq.) (500 ML) at room temperature. The aqueous layer was extracted with EtOAc (2×100 mL). The resulting mixture was concentrated under vacuum. The residue was purified by silica gel column chromatography, eluted with PE/EA (5:1) to afford 326-1 (12 g, 73.96%) as a yellow oil.

### 2. Synthesis of 326-2

To a stirred solution of 326-1 (11.5 g, 51.976 mmol, 1 equiv) in DCM (200 mL) was added Dess-Martin (26.45 g, 62.371 mmol, 1.2 equiv) at 0° C. under nitrogen atmosphere. The resulting mixture was stirred for 2 h at room temperature. The reaction mixture was washed with water (100 mL). The resulting mixture was concentrated under vacuum. The residue was purified by silica gel column chromatography, eluted with PE/EA (10:1) to afford 326-2 (4 g, 31.59%) as a brown oil.

### 3. Synthesis of 326-3

To a stirred solution of 326-2 (4 g, 18,245 mmol, 1 equiv) and nitroethane (4.11 g, 54.735 mmol, 3 equiv) in DMF (100 mL) was added NH40Ac (1.41 g, 18.245 mmol, 1 equiv), HOAc (0.55 g, 9.123 mmol, 0.5 equiv) and NaN3 (2.97 g, 45,613 mmol, 2.5 equiv) at room temperature under nitrogen atmosphere. The resulting mixture was stirred overnight at 100° C. The reaction was quenched with NaHCO₃ (aq.) (500 mL) at room temperature. The aqueous layer was extracted with EtOAc (3×100 mL). The resulting mixture was concentrated under vacuum. The residue was purified by silica gel column chromatography, eluted with PE/EA (2:1) to afford 326-3 (1.3 g, 23.55%) as a yellow oil.

### 4. Synthesis of 326-4

To a stirred solution of 326-3 (1.2 g, 4.407 mmol 1 equiv) and (Boc)20 (2.40 g, 11.018 mmol, 2.5 equiv) in DCM (100 mL) was added TEA. (1.34 g, 13.221 mmol, 3 equiv) at room temperature. The resulting mixture was stirred overnight at room temperature. The resulting mixture was concentrated under vacuum. The residue was purified by silica gel column chromatography, eluted with PE/EA (10:1) to afford 326-4 (1.1 g, 62.33%) as a yellow oil.

### 5. Synthesis of 326-5

To a solution of 326-4 (1 g, 2.685 mmol, 1 equiv) in MeOH (30 mL) was added Pd/C (0.1 g, 10%) under nitrogen atmosphere in a 100 ml round-bottom flask. The mixture was hydrogenated at room temperature for 3 h under hydrogen atmosphere using a hydrogen balloon, filtered through a Celite pad and concentrated under reduced pressure. The crude product was used in the next step directly without further purification. This resulted in 326-5 (800 mg, 60.90%) as a yellow oil.

### 6. Synthesis of 326-6

To a stirred solution of 326-5 (800 mg, 1.635 mmol, 1 equiv, 70%) and I-2 (702.28 mg, 2.453 mmol, 1.5 equiv) in DCE (5 mL) was added STAB (693.17 mg, 3.270 mmol, 2 equiv) at room temperature. The resulting mixture was stirred for 3 h at room temperature. The reaction was quenched with NaHCO3 (aq.) (100 mL) at room temperature. The aqueous layer was extracted with DCM (2×100 mL). The resulting mixture was concentrated under vacuum. The residue was purified by Prep-TLC (DCM/MeOH 40:1) to afford 326-6 (520 mg, 48.26%) as a yellow solid.

### 7. Synthesis of 326-7

To a stirred solution of 326-6 (500 mg, 0.816 mmol, 1 equiv) and Pyridine (645.46 mg, 8.160 mmol, 10 equiv) in DCM (10 mL) was added Triphosgene (121.07 mg, 0.408 mmol, 0.5 equiv) at room temperature. The resulting mixture was stirred for 10 min at room temperature. The reaction was quenched with saturated NaHCO₃ (aq.) (100 mL) at room temperature. The aqueous layer was extracted with DCM (2×30 mL). The resulting mixture was concentrated under vacuum. This resulted in 326-7 (400 mg, 61.40%) as a yellow solid.

### 8. Synthesis of 326-8

To a stirred solution of 326-7 (380 mg, 0.595 mmol, 1 equiv) in DCM (5 mL) was added TFA (1 mL) at room temperature. The resulting mixture was stirred for 2 h at room temperature. The resulting mixture was concentrated under vacuum. The crude product was purified by Prep-HPLC with the following conditions (Column: XBridge Shield RP18 OBD Column, 30*150 mm, 5 μm; Mobile Phase A: Water (10 mmol/L NH4HCO3+0.1% NH3·H2O). Mobile Phase B: ACN; Flow rate: 60 mL/min; Gradient: 53% B to 78% B in 8 min, Wave Length: 220 am; RT1 (min): 7.72) to afford 326-8 (180 ag, 55.05%) as a yellow solid.

### 9. Synthesis of 326

The 326-8 (180 mg) was purified by Prep-Chiral-HPLC with the following conditions (Column: CHIRALPAK 10, 2*25 cm, 5 μm; Mobile Phase A: Hex (0.5% 2M NH3-MeOH), Mobile Phase B: EtOH:DCM=1:1; Flow rate: 20 mL/min; Gradient: 15% B to 15% B in 16 min; Wave Length: 220/254 nm; RT1 (min): 8.94; RT2 (min): 12.89; the first peak is product) to afford 326 (67.1 mg, 37:20%) as a yellow solid.

LC-MS: (ES, m/z): [M+H]+ 539. H-NMR: (400 MHz, DMSO-d6, δ ppm): 0.89-0.99 (m, 4H), 1.46-1.72 (m, 6H), 1.72-1.96 (m, 6H), 2.13 (s, 3H), 2.74-2.85 (m, 2H), 3.05-3.25 (m, 1H), 3.24 (s, 2H), 4.07-4.09 (m, 1H), 7.00 (s, 1H), 7.21-7.30 (m, 2H), 7.36-7.42 (m, 1H), 7.61-7.65 (m, 2H), 7.75 (s, 1H), 14.27-14.77 (m, 1H).

### Example 327. Synthesis of Compound 327

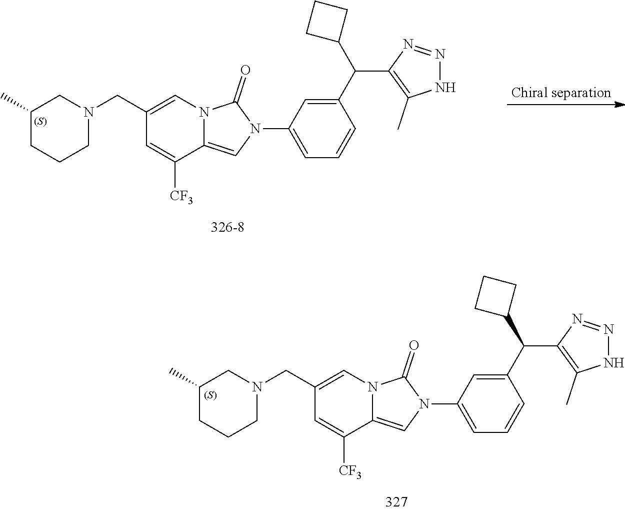

### 1. Synthesis of 327

326-8 (180 mg) was purified by Prep-Chiral-HPLC with the following conditions (Column: CHIRALPAK 10, 2*25 cm, 5 μm; Mobile Phase A: Hex (0.5% 2M NH3-MeOH), Mobile Phase B: EtOH:DCM=1:1; Flow rate: 20 L/min; Gradient: 15% B to 15% B in 16 rain; Wave Length: 220/254 nm; RT1 (min): 8.94; RT2 (min): 12.89; the second peak is product) to afford 327 (70.8 mg, 39.18%) as a yellow solid.

LC-MS: (ES, m/z): [M+H]+ 539. H-NMR: (400 MHz, DMSO-d6, δ ppm): 0.89-0.99 (m, 4H), 1.41-1.49 (m, 1H), 1.49-1.72 (m, 5H), 1.72-1.95 (m, 6H), 2.12 (s, 3H), 2.73-2.83 (m, 2H), 3.08-3.23 (m, 1H), 3.24 (s, 2H), 4.07-4.09 (m, 1H), 7.00 (s, 1H), 7.25-7.30 (m, 2H), 7.38-7.42 (m, 1H), 7.61-7.65 (m, 2H), 7.75 (s, 1H), 14.29-14.69 (m, 1H).

### Example 328. Synthesis of Compound 328

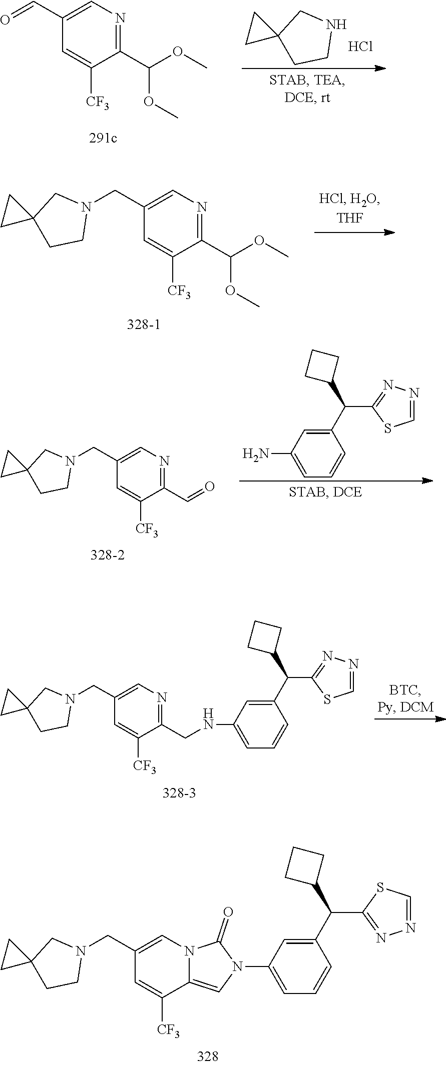

### 1. Synthesis of 328-1

To a stirred solution of 5-azaspiro [2.4] heptane hydrochloride (375 mg, 2.80 mmol, 1.0 equiv) and 291c (700 mg, 2.80 mmol, 1.0 equiv) in DCE (7 mL) were added TEA (284 mg, 2.80 mmol, 1.0 equiv) and STAB (1190 mg, 5.61 mmol, 2.0 equiv) at room temperature. The resulting mixture was stirred for 2 h at room temperature. The reaction was quenched with sat. NH4Cl (aq.) at room temperature. The resulting mixture was extracted with CH2Cl2 (3×20 mL). The combined organic layers were concentrated under reduced pressure. The residue was purified by Prep-TLC (PE/EA=1:1) to afford 328-1 (300 mg, 31%) as a colorless oil.

### 2. Synthesis of 328-2

A solution of 328-1 (300 mg, 0.90 mmol, 1.0 equiv) and in THF (0.7 mL) and HC (3 mL, 98.74 mmol, 1 M) was stirred for 2 h at room temperature. The residue was neutralized to pH 7 with saturated NaHCO₃ (aq.). The aqueous layer was extracted with CH2Cl2 (3×20 mL). The combined organic layers were concentrated under reduced pressure. The residue was purified by Prep-TLC (PE/EA=1:1) to afford 328-2 (190 mg, 72%) as a colorless oil.

### 3. Synthesis of 328-3

A solution of 328-2 (256 mg, 0.90 mmol, 1.3 equiv) in DCE (1.7 mL) was treated with (R)-3-(cyclobutyl(1,3,4-thiadiazol-2-yl)methyl)aniline (170 mg, 0.69 mmol, 1.0 equiv) for 0.5 h at room temperature followed by the addition of STAB (293 mg, 1.38 mmol, 2.0 equiv) at room temperature. The resulting mixture was stirred for overnight at room temperature. The reaction was quenched with sat. NH4Cl (aq.) at room temperature. The aqueous layer was extracted with CH2Cl2 (3×20 mL). The resulting mixture was concentrated under reduced pressure. The residue was purified by Prep-TLC (PE/EA=1:1) to afford 328-3 (300 mg, 71%) as a yellow oil.

### 4. Synthesis of 328

To a stirred solution of 328-3 (300 mg, 0.58 mmol, 1 equiv) and pyridine (277 mg, 3.50 mmol, 6.0 equiv) in DCM (3 mL) were added triphosgene (60 mg, 0.20 mmol, 0.35 equiv) at 0° C. The resulting mixture was stirred for 1.5 h at 0° C. The reaction was quenched with sat, NaHCO₃ (aq.) (10 mL) at room temperature. The resulting mixture was extracted with CH2Cl2 (3×20 mL). The resulting mixture was concentrated under reduced pressure. The residue was purified by reverse flash chromatography with the following conditions: column, C18 silica gel; mobile phase, MeCN in water (10 mmol/L NH4HCO3) 15% to 85% gradient in 40 min; UV 254 nm. This resulted in 328 (109.3 mg, 34%) as a yellow solid.

LC-MS: (ES, m/z): [M+H]+ 540. H-NMR: (400 MHz, DMSO-d6, δ ppm): 0.45-0.50 (m, 4H), 1.73-1.77 (m, 3H), 1.79-1.87 (m, 4H), 2.04-2.08 (m, 1H), 2.45 (s, 2H), 2.66-2.69 (t, 2H), 3.21-3.24 (m, 1H), 3.39 (s, 2H), 4.71-4.73 (d, 1H), 7.04 (s, 1H), 7.33-7.37 (m, 2H), 7.46-7.48 (t, 1H), 7.68 (s, 1H), 7.75-7.77 (m, 1H), 7.86 (s, 1H), 9.50 (s, 1H).

### Example 329. Synthesis of Compound 329

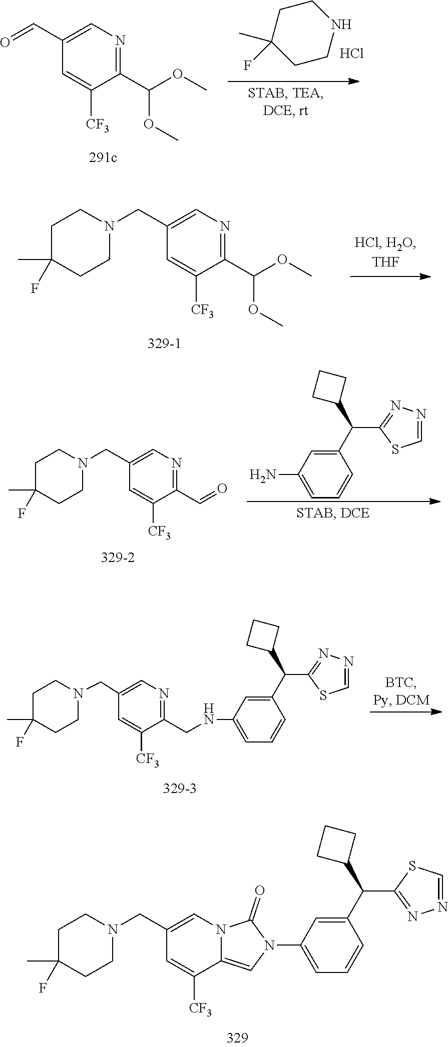

### 1. Synthesis of 329-1

To a stirred solution of 4-fluoro-4-methylpiperidine hydrochloride (500 mg, 3.25 mmol, 1.0 equiv) and 291c (811 mg, 3.25 mmol, 1.0 equiv) in DCE (5 mL) were added TEA (658 mg, 6.51 mmol, 2.0 equiv) and STAB (1379 mg, 6.51 mmol, 2.0 equiv) at room temperature. The resulting mixture was stirred for overnight at room temperature. The reaction was quenched with water at room temperature. The resulting mixture was extracted with CH2Cl2 (3×20 ml). The combined organic layers were concentrated under reduced pressure. The residue was purified by Prep-TLC (PE/EA=1:1) to afford 329-1 (360 mg, 32%) as a colorless oil,

### 2. Synthesis of 329-2

To a stirred solution of 329-1 (350 mg, 0.99 mmol, 1.0 equiv) in THF (3.5 mL) was added HCl (3.5 mL, 1M) at room temperature. The recruiting mixture was stirred for 2 h at 80° C. The mixture was neutralized to pH 7 with saturated NaHCO₃ (aq.). The resulting mixture was extracted with CH2Cl2 (3×20 mL). The combined organic layers were concentrated under reduced pressure. The residue was purified by Prep-TLC (PE/EA=1:1) to afford 329-2 (300 mg, 97%) as a colorless oil.

### 3. Synthesis of 329-3

To a stirred solution of 291-2 (200 mg, 0.81 mmol, 1.0 equiv) and 329-2 (322 mg, 1.09 mmol, 1.3 equiv) in DCE (4 mL) was added STAB (345 mg, 1.63 mmol, 2.0 equiv) at room temperature. The resulting mixture was stirred for overnight at room temperature. The reaction was quenched with water (15 mL) at room temperature. The resulting mixture was extracted with CH2Cl2 (3×20 mL). The combined organic layers were concentrated under reduced pressure. The residue was purified by Prep-TLC (PE/EA=1:1) to afford 329-3 (350 mg, 80%) as an off-white solid.

### 4. Synthesis of 329

To a stirred solution of 329-3 (350 mg, 0.66 mmol, 1.0 equiv) and pyridine (311 mg, 3.96 mmol, 6.0 equiv) in DCM (3.5 mL) was added triphosgene (68 mg, 0.23 mmol, 0.35 equiv) at 0° C. The resulting mixture was stirred for 30 min at 0° C. The reaction was quenched with sat. NaHCO₃ (aq.) at room temperature. The resulting mixture was extracted with CH2Cl2 (3×20 mL). The combined organic layers were concentrated under reduced pressure. The residue was purified by reverse flash chromatography with the following conditions: column, C18 silica gel; mobile phase, acetonitrile in water (0.1% NH4HCO3), 20% to 90% gradient in 40 min; UV 254 nm. This resulted in 329 (106 mg, 28%) as a yellow solid.

LC-MS: (ES, m/z): [M+H]+ 560. H-NMR: (400 MHz, dmso-d6, δ ppm): 1.29-1.34 (d, 3H), 1.58-1.67 (m, 6H), 1.71-1.88 (m, 4H), 2.05-2.06 (m, 1H), 2.24-2.29 (m, 2H), 2.60-2.63 (m, 2H), 3.20-3.23 (m, 1H), 3.26 (s, 2H), 4.71-4.73 (d, 1H), 7.01 (s, 1H), 7.33-7.37 (m, 2H), 7.46-7.50 (m, 1H), 7.69 (s, 1H), 7.75-7.77 (m, 1H), 7.86 (s, 1H) 9.51 (s, 1H).

### Example 330. Synthesis of Compound 330

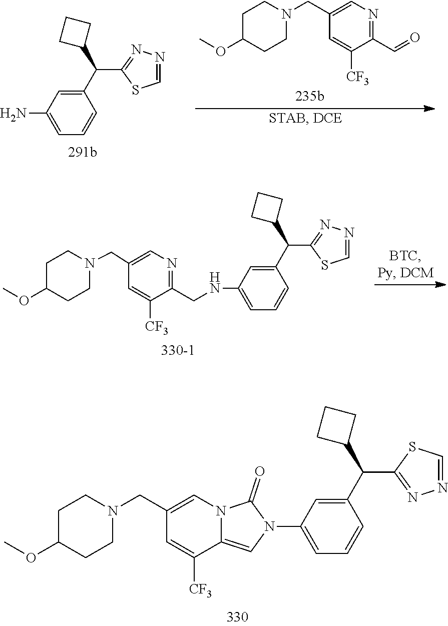 [SMILES: Nc1cccc([C@H](c2nncs2)C2CCC2)c1 ; COC1CCN(Cc2cnc(C=O)c(C(F)(F)F)c2)CC1 ; COC1CCN(Cc2cnc(CNc3cccc([C@H](c4nncs4)C4CCC4)c3)c(C(F)(F)F)c2)CC1 ; COC1CCN(Cc2cc(C(F)(F)F)c3cn(-c4cccc([C@H](c5nncs5)C5CCC5)c4)c(=O)n3c2)CC1]

### 1. Synthesis of 330-1

A solution of 291b (200 mg, 0.815 mmol, 1 equiv) in DCE (2 mL) was treated with 235b (295.72 mg, 0.978 mmol, 12 equiv) for 2 h at room temperature followed by the addition of STAB (345.54 mg, 1.630 mmol, 2 equiv) at room temperature. The resulting mixture was stirred overnight at room temperature. The reaction was quenched by the addition of sat. NaHCO₃ (aq.) (8 mL) at room temperature. The resulting mixture was extracted with DCM/MeOH (10:1)(30 mL) The resulting mixture was concentrated under reduced pressure. The residue was purified by reverse flash chromatography with the following conditions: column, C18 silica gel; mobile phase A: Water (0.1% NH4HCO3); mobile phase B: ACN, Gradient: 25% B to 80% B in 30 min; detector, UV 254 nm. This resulted in 330-1 as a yellow solid.

### 2. Synthesis of 330

A solution of 330-1 (353 mg, 0.663 mmol, 1 equiv) in DCM (10 mL) was treated with Pyridine (314.54 mg, 3.978 mmol, 6 equiv) at room temperature followed by the addition of triphosgene (78.63 mg, 0,265 mmol, 0.4 equiv) at 0° C. The resulting mixture was stirred for 30 min at room temperature. The reaction was quenched by the addition of sat. NaHCO₃ (aq.) (8 mL) at room temperature. The resulting mixture was extracted with DCM/MeOH (10:1)(30 mL). The combined organic layers were concentrated under reduced pressure. The residue was purified by reverse flash chromatography with the following conditions: column, C18 silica gel; mobile phase A: Water (0.1% NH4HCO3); mobile phase B: ACN, Gradient: 25% B to 80% B in 30 min; detector, UV 254 nm. The crude product (190 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 42% B to 72% B in 8 min, 72% B; Wave Length: 220 urn) to afford 330 (120.3 mg, 31.25%) as a yellow solid. LC-MS-330 (ES, m/z): [M+H]+558. H-NMR: (400 MHz, DMSO, ppm): 51.41-1.43 (m, 1H), 1.77-1.87 (m, 71H), 2.07-2.11 (mu, 3H), 3.22-3.28 (m, 8H), 4.71-4.74 (d, 1H), 7.02 (s, 1H), 7.33-7.35 (m, 2H), 7.46-7.51 (t, 1H), 7.67 (s, 1H), 7.75-7.77 (s, 1H), 7.86 (s, 1H), 9.51 (s, 1H).

### Example 331. Synthesis of Compound 331

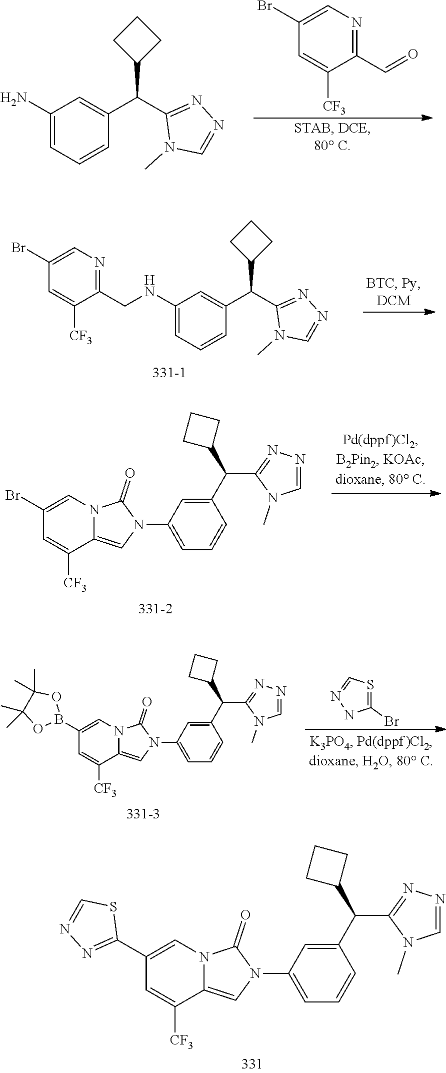

### 1. Synthesis of 331-1

Into a 1 mL 3-necked round-bottom flask were added 244b (g, 4127 mmol, 1 equiv), DCE (10 mL), I-2 (1.57 g, 6.191 mol, equiv) and STAB (2.62 g, 12.381 mmol, 3 equiv) at room temperature. The resulting mixture was stirred for 2 h at 80° C. The mixture was acidified to pH 7 with saturated NH₄Cl (aq.) The resulting mixture was extracted with CH₂Cl₂ (3×20 mL). The resulting mixture was concentrated under reduced pressure. The residue was purified by Prep-TLC (CH₂Cl₂/MeOH 20:1) to afford 331-1 (1.1 g, 52.72%) as a light brown oil.

### 2. Synthesis of 331-2

Into a 100 mL round-bottom flask were added 331-1 (1 g, 2.082 mmol, equiv), DCM (20 mL) and Pyridine (0.99 g, 12.492 mmol, 6 equiv) at 0° C. To the above mixture was added Triphosgene (0.28 g, 0.937 mmol 0.45 equiv) at 0° C. The resulting mixture was stirred for additional 8 min at 0° C. The reaction was quenched by the addition of sat. NaHCO₃ (aq.) (10 mL) at room temperature. The resulting mixture was extracted with CH₂C2 (3×20 mL). The resulting mixture was concentrated under reduced pressure. The residue was purified by Prep-TLC (CH₂Cl₂/MeOH 20:1) to afford 331-2 (0.8 g, 73.62%) as a light yellow solid.

### 3. Synthesis of 331-3

Into 100 mL round-bottom flask were added 331-3 (800 & 1.580 mml, 1 equiv), dioxane (10 mL), KOAc (310.13 mg, 3.160 mmol, 2 equiv), 4,4,5,5-tetramethyl-2-(tetramethyl-1,3,2-dioxaborolan-2-yl)1,3,2-dioxaborolane (1.60 g, 6.320 mmol, 4 equiv) and Pd(dppf)Cl₂ (173.42 mg, 0.237 mmol, 0.15 equiv) at room temperature. The resulting mixture was stirred for 8 hr at 810° C.; under nitrogen atmosphere. The resulting mixture was diluted with water (15 mL). The aqueous layer was extracted with CH₂Cl₂ (3×10 mL). The resulting mixture was concentrated under vacuum. The residue was purified by Prep-TLC (CH₂Cl₂/MeOH 15:1) to afford 331-3 (300 mg, 32.60%) as a light yellow oil.

### 4. Synthesis of 331

Into an 8 mL sealed tube were added 331-3 (200 mg, 0.361 mmol, 1 equiv), 2-bromo-1,3,4-thiadiazole (89.45 mg, 0.541 mmol, 1.5 equiv), K₃PO₄ (230.14 mg, 1.083 mmol, 3 equiv), dioxane (1.6 mL) and H₂O (0.4 mL) at room temperature. To the above mixture was added Pd(dppf)Cl₂ (52.89 mg, 0.072 mmol, 0.2 equiv) at room temperature. The resulting mixture was stirred for 6 h at 80° C. under nitrogen atmosphere. The resulting mixture was diluted with water (5 mL). The aqueous layer was extracted with CH₂Cl₂ (3×5 mL). The resulting mixture was concentrated under vacuum. The residue was purified by Prep-TLC (Cl2Cl₂/MeOH 15:1) to afford crude product (70 mg) as a light yellow oil. The crude product (70 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 37% B to 67% B in 8 min; Wave Length: 220 nm; RT (min): 7.17) to afford 331 (44.4 mg, 24.02%) as a light yellow solid.

LCMS: (ES, m/z): [M+H]⁺ 512. H-NMR: (300 MHz, DMSO-d6, ppm): δ 1.67-1.99 (m, 5H), δ 2.05-2.11 (m, 1H), δ 3.22-3.26 (m, 1H), δ 3.44 (s, 3H), δ 4.27-4.30 (d, 1H), δ 7.23-7.25 (d, 1H), δ 7.46-7.48 (d, 1H), δ 7.50 (s, 1H), δ 7.61 (s, 1H), δ 7.71-7.73 (d, 1H), δ 7.77 (s, 1H), δ 8.34 (s, 1H), δ 8.46 (s, 1H), δ 9.63 (s, 1H).

### Example 332. Synthesis of Compounds 332_P1 and P2

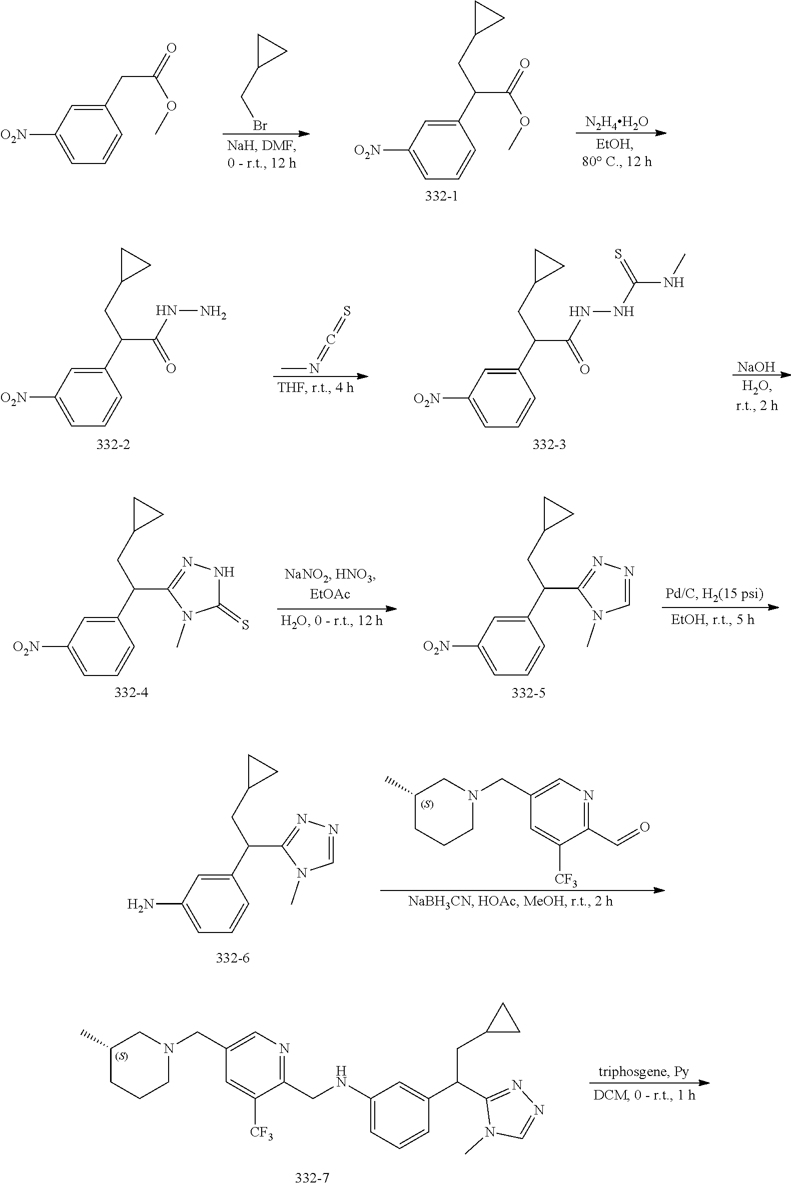

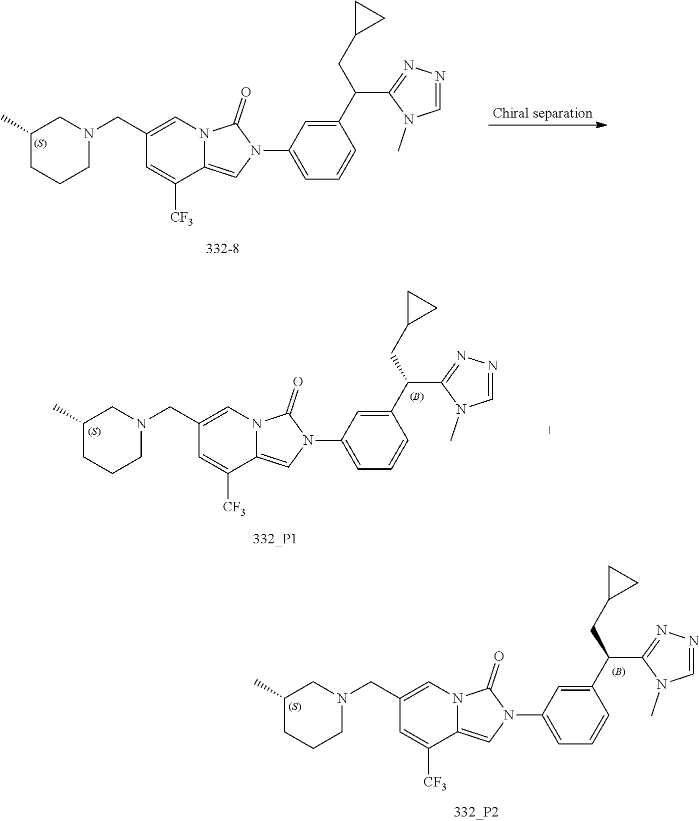

### 1. Synthesis of 332-1

To a solution of NaH (3.38 g, 84.54 mmol, 60% purity, 1.1 equiv) in DMF (150 mL) was added a solution of methyl 2-(3-nitrophenyl)acetate (15 g, 76.86 mmol, 1 equiv) in DMF (20 mL) under nitrogen atmosphere, the mixture was cooled to 0° C. Then bromomethylcyclopropane (11 ml, 115 mmol, 1.5 equiv) was added to the mixture at 0° C., the result mixture was stirred at 25° C. for 12 hr under nitrogen atmosphere. The reaction mixture was diluted with Sat.NH4Cl (150 mL), extracted with EtOAc (150 mL×3). The combined organic layers were dried over anhydrous Na2SO4, filtered and the filtrate was concentrated in vacuum. The residue was purified by silica gel column chromatography eluted with PE/EtOAc (3:1) to afford 332-1 (13 g, 68%) as a light yellow solid.

H-NMR-332-1: (400 MHz, CHLOROFORM-d) δ 8.18 (t, J=2.0 Hz, 1H), 8.12-8.07 (m, 1H), 7.65 (d, J=8.0 Hz, 1H), 7.47 (t, J=8.0 Hz, 1H), 3.77 (t, J=7.6 Hz, 1H), 3.68-3.65 (m, 3H), 1.92-1.85 (m, 1H), 1.83-1.74 (m, 1H), 0.61-0.50 (m, 1H), 0.44-0.35 (m, 2H), 0.12-0.04 (m, 1H), −0.03-−0.07 (m, 1H)

### 2. Synthesis of 332-2

To a solution of 332-1 (12 g, 48.1 mmol, 1 equiv) in EtOH (120 mL) was added N2H4·H2O (38.2 mL, 770 mmol, 98% purity, 16 equiv). The mixture was stirred at 80° C. for 12 h. The reaction mixture was concentrate in vacuum to remove EtOH (120 mL), then the mixture was diluted with water (600 mL), and extracted with EtOAc (200 mL×3). The combined organic layers were dried over anhydrous Na2SO4, filtered and the filtrate was concentrated in vacuum to afford 332-2 (12 g, crude) as a yellow solid.

### 3. Synthesis of 332-3

To a solution of 332-2 (3.23 g, 12.96 mmol, 1 equiv) in THF (40 mL) was added methylimino(thioxo)methane (1.77 mL, 25.9 mmol, 2 equiv). The mixture was stirred at 20° C. for 4 hr. The reaction mixture was diluted water (40 mL), extracted with EtOAc (40 mL×3). The combined organic layers were dried over anhydrous Na2SO4, filtered and concentrated in vacuum to afford 332-3 (3.7 g, crude) as a white solid.

### 4. Synthesis of 332-4

To a solution of NaOH (3.67 g, 91.8 mmol, 8 equiv) in water (37 mL) was added 332-3 (3.7 g, 11.4 mmol, 1 equiv). The mixture was stirred at 25° C. for 2 hr. The reaction mixture was diluted with water (40 mL), and adjusted pH to 3 by 1N HCl. Then the mixture was filtered and the filter cake was concentrated in vacuum to afford 332-4 (3.47 g, 98%) as a white solid.

H-NMR-332-4: (400 MHz, DMSO-d6) δ 13.68 (s, 1H), 8.15-8.04 (m, 2H), 7.70 (d, J=8.0 Hz, 1H), 7.63-7.55 (m, 1H), 4.45 (t, J=7.6 Hz, 1H), 3.22 (s, 3H), 2.04-1.88 (m, 1H), 1.79-1.68 (m, 1H), 0.59-0.46 (m, 1H), 0.33-0.18 (m, 2H), 0.06-0.03 (m, 1H), −0.06-0.14 (m, 1H)

### 5. Synthesis of 332-5

HNO3 (7.55 mL, 114 mmol, 68% purity, 10 equiv) was added to water (105.6 m) to afford the diluted HNO3 solution (1 M, 113 mL). To a solution of 341-4 (3.47 g, 11.4 mmol, 1 equiv) and NaNO2 (7.87 g, 114 mmol, 10 equiv) in EtOAc (3.5 mL, 35.7 mmol, 3.14 equiv) and water (35 mL) was added diluted HNO3 solution (1 M, 113 mL) dropwise at 0° C. Then the mixture was stirred at 25° C. for 12 hr. The reaction was quenched by the addition of Sat.NaHCO3 (100 mL) The aqueous phase was extracted with CH2Cl2 (100 ml×3). The combined organic layers were dried over anhydrous Na2SO4, filtered and the filtrate was concentrated in vacuum to afford 332-5 (2.7 g, crude) as a yellow oil.

### 6. Synthesis of 332-6

To a 332-5 (2.5 g, 9.18 mmol, 1 eq) in EtOH (30 mL) was added Pd/C (2.5 g, 10% purity). The mixture was stirred at r.t. for 5 hr under H2 (15 psi). The reaction mixture was filtered through celite, and the filtrate was concentrated in vacuum o afford 332-6 (1.9 g, 88.24%) as a gray solid, H-NMR-332-6: (400 MHz, DMSO-d6) δ 8.26 (s, 1H), 6.87 (t, J=8.0 Hz, 1H), 6.42-6.28 (m, 311), 4.96 (s, 2H), 3.94 (t, J=7.6 Hz, 1H), 3.32 (s, 311), 2.12-1.93 (m, 1H), 1.84-1.63 (m, 1H), 0.72-0.50 (in, 1H), 0.41-0.21 (m, 2H), −0.05-0.09 (m, 2H).

### Example 333. Synthesis of Compound 333

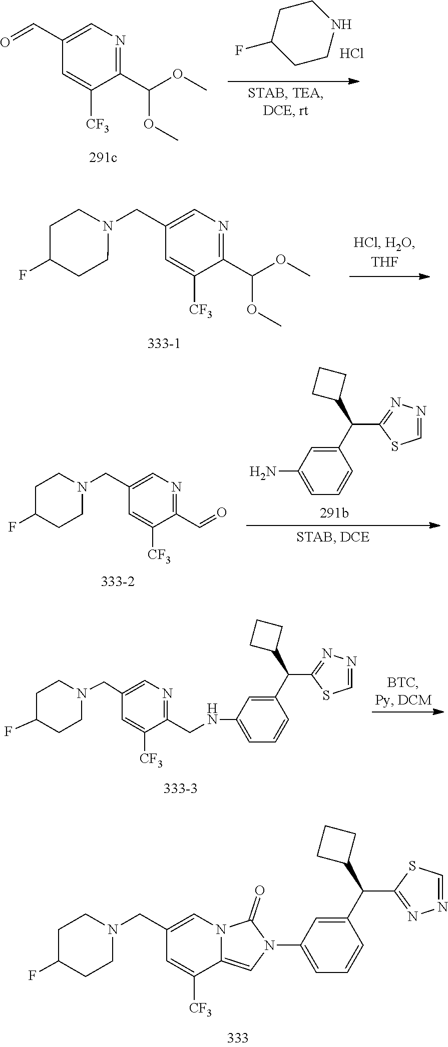

### 1. Synthesis of 333-1

A solution of 291c (800 mg 3,210 mmol, 1 equiv) in DCE (8 mL) was treated with 4-fluoropiperidine hydrochloride (896.34 mg, 6.420 mmol, 2 equiv) and TEA (974.61 mg, 9.630 mmol, 3 equiv) for 2 h at room temperature followed by the addition of STAB (1360.83 mg, 6,420 mmol, 2 equiv) at room temperature. The resulting mixture was stirred for overnight at room temperature. The reaction was quenched with sat. NaH4Cl (aq.) at room temperature. The resulting mixture was extracted with EtOAc (3×20 mL). The combined organic was concentrated under reduced pressure. The residue was purified by Prep-TLC (PE/EA=3:1) to afford 333-1 (550 mg, 48.90%) as a off-white oil.

### 2. Synthesis of 333-2

To a stirred solution of 333-1 (550 mg, 1.635 mmol, 1 equiv) in TIE (5.5 mL) was added HCl (5.5 ml, 1M) at room temperature. The resulting mixture was stirred for 2 h at 80° C. The mixture was neutralized to pH 7 with saturated NaHCO₃ (aq.). The resulting mixture was extracted with EtOAc (3×20 mL). The combined organic was concentrated under reduced pressure. The residue was purified by Prep-TLC (PE/EA=2:1) to afford 333-2 (442 mg, 90.33%) as a colorless oil.

### 3. Synthesis of 333-3

A solution of 333-2 (247:27 mg, 0.851 mmol, 1.1 equiv) in DCE (2 mL) was treated with 291b (190 mg, 0.774 mmol, 1.00 equiv) for 2 h at room temperature under nitrogen atmosphere followed by the addition of STAB (328.27 mg, 1.548 mmol, 2 equiv) at room temperature. The resulting mixture was stirred for overnight at room temperature. The reaction was quenched with sat. NaHCO₃ (aq.)(8 ml) at room temperature. The resulting mixture was extracted with EtOAc (3×20 mL). The combined organic was concentrated under reduced pressure. The residue was purified by reverse flash chromatography with the following conditions: column, C18 silica gel; mobile phase A: Water (0.1% NH4HCO3); mobile phase B: ACN, Gradient: 20% B to 80% B in 30 min; detector, UV 254 nm. This resulted in 333-3 (270 mg, 63.07%) as a light yellow solid,

### 4. Synthesis of 333

A solution of 333-3 (270 mg, 0.520 mmol, 1 equiv) in DCM (8 ml) was treated with Pyridine (246.61 mg, 3,120 mmol, 6 equiv) at room temperature then followed by the addition of triphosgene (61.72 mg, 0.208 mmol, 0.4 equiv) at 0° C. The resulting mixture was stirred for 30 min at room temperature. The reaction was quenched by the addition of sat. NaHCO₃ (aq.) (8 mL) at room temperature. The resulting mixture was extracted with DCM/MeOH (10:1)(30 mL). The combined organic layers were concentrated under reduced pressure. The residue was purified by Prep-HPLC with the following conditions (Column: XBridge Shield RP8 OBD Column, 30*150 mm, 5 um; Mobile Phase A: Water (10 mmol/l NH4HCO3), Mobile Phase B: MeOH; Gradient: 64% B to 82% B in 8 min, 82% B; Wave Length: 220 nm) to afford 333 (100.8 mg, 35.55%) as a yellow solid.

LC-MS: (ES, m/z): [M+H]+ 546. H-NMR: (400 MHz, DMSO, ppm): 51.75-2.05 (m, 9H), 2.06-2.08 (m, 1H), 2.33-2.35 (m, 2H), 2.51-2.55 (m, 2H), 3.21-3.23 (m, 1H), 3.25 (s, 2H), 4.63-4.77 (m, 2H), 7.02 (s, 1H), 7.33-7.37 (m, 2H), 7.46-7.50 (t, 1H), 7.69 (s, 1H), 7.75-7.77 (m, 1H), 7.86 (s, 1H), 9.50 (s, 1H).

### Example 334. Synthesis of Compound 334

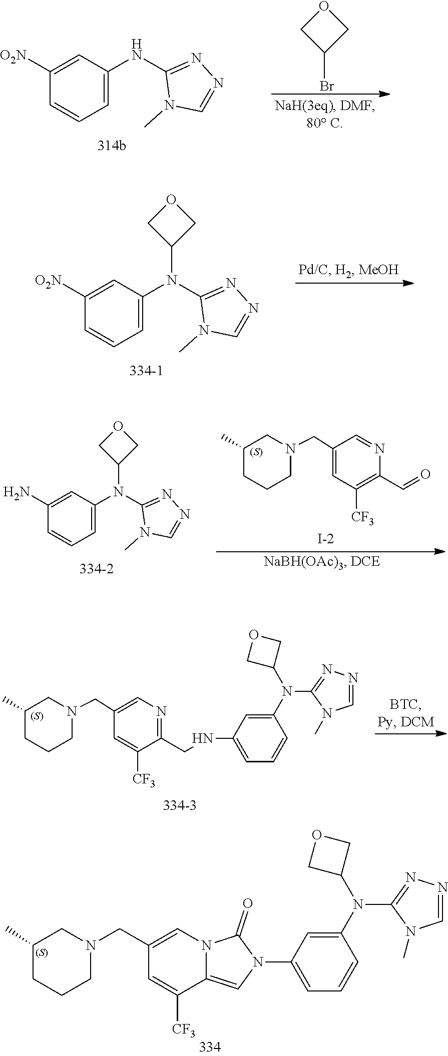

### 1. Synthesis of 334-1

To a stirred solution of 314b (2 g, 9.124 mmol, 1 equiv) and DMF (20 mL) was added NaH (1.09 g, 27.372 mmol, 3 equiv, 60%) at room temperature. The mixture was stirred for 1 h at room temperature. To the above mixture was added 3-bromooxetane (3.75 g, 27.372 mmol, 3 equiv) at room temperature. The resulting mixture was stirred for overnight at 80° C. The reaction was quenched with sat. NH4Cl (aq.) (100 ml) at room temperature. The aqueous layer was extracted with EtOAc (3×40 mL). The resulting mixture was concentrated under vacuum. The residue was purified by Prep-TLC (DCM/MeOH=40:1) to afford 334-1 (300 mg, 10.99%) as a brown oil,

### 2. Synthesis of 334-2

To a solution of 334-1 (300 mg, 1.090 mmol, 1 equiv) in 10 ml MeOH was added Pd/C (20%, 60 mg) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure. The residue was purified by Prep-TLC (DCM/MeOH=20:1) to afford 334-2 (170 mg, 59.78%) as a colorless oil.

### 3. Synthesis of 334-3

Into a 25 mL round-bottom flask were added 334-2 (170 mg, 0.693 mmol, 1 equiv), I-2 (238.11 mg, 0.832 mmol, 1.2 equiv), STAB (293.78 mg, 1.386 mmol, 2 equiv) and DCE (5 mL) at room temperature. The resulting mixture was stirred for overnight at room temperature. The reaction was quenched with sat. NH4Cl (aq.) (50 mL) at room temperature. The aqueous layer was extracted with EtOAc (3×20 mL). The resulting mixture was concentrated under vacuum. The residue was purified by Prep-TLC (DCM/MeOH=30:1) to afford 334-3 (160 mg, 42.98%) as a brown oil,

### 4. Synthesis of 334

Into a 50 mL round-bottom flask were added 334-3 (170 mg, 0.330 mmol, 1 equiv), pyridine (208.65 mg, 2.640 mmol, 8 equiv) and DCM (10 mL) at room temperature. To the above mixture was added Triphosgene (68.49 mg, 0.231 mmol, 0.7 equiv) at 0° C. The resulting mixture was stirred for additional 5 min at 0° C. The reaction was quenched with sat. NaHCO₃ (aq.) (30 mL) at room temperature. The aqueous layer was extracted with EtOAc (3×10 mL). The resulting mixture was concentrated under vacuum. The crude product (200 mg) was purified by Prep-HPLC with the following conditions (Column: X Bridge Prep OBD C18 Column, 30*150 mm, 5 μm; Mobile Phase A: water (10 mmol/L. NH4HCO3), Mobile Phase B: ACN; Flow rate: 60 mL/min; Gradient: 39% B to 69% B in 8 min, 69% B; Wave Length: 220 nm;

H-NMR: 1H NMR (400 MHz, CD3OD) δ 0.89-1.02 (m, 4H), δ 1.60-1.78 (m, 5H), δ 1.95-2.00 (m, 1H), δ 2.82-2.91 (m, 2H), δ 3.33 (s, 2H), δ 3.52 (s, 3H), δ 4.63-4.66 (m, 2H), δ 4.89-4.95 (m, 2H), δ 5.19-5.26 (m, 1H), δ 6.68-6.71 (d, 1H), δ 7.12 (s, 1H), δ 7.17 (s, 1H), δ 7.29 (s, 1H), δ 7.34-7.36 (d, 1H), δ 7.47-7.42 (m, 1H), δ 7.66 (s, 1H), δ 8.51 (s, 1H).

### Example 335. Synthesis of Compound 335

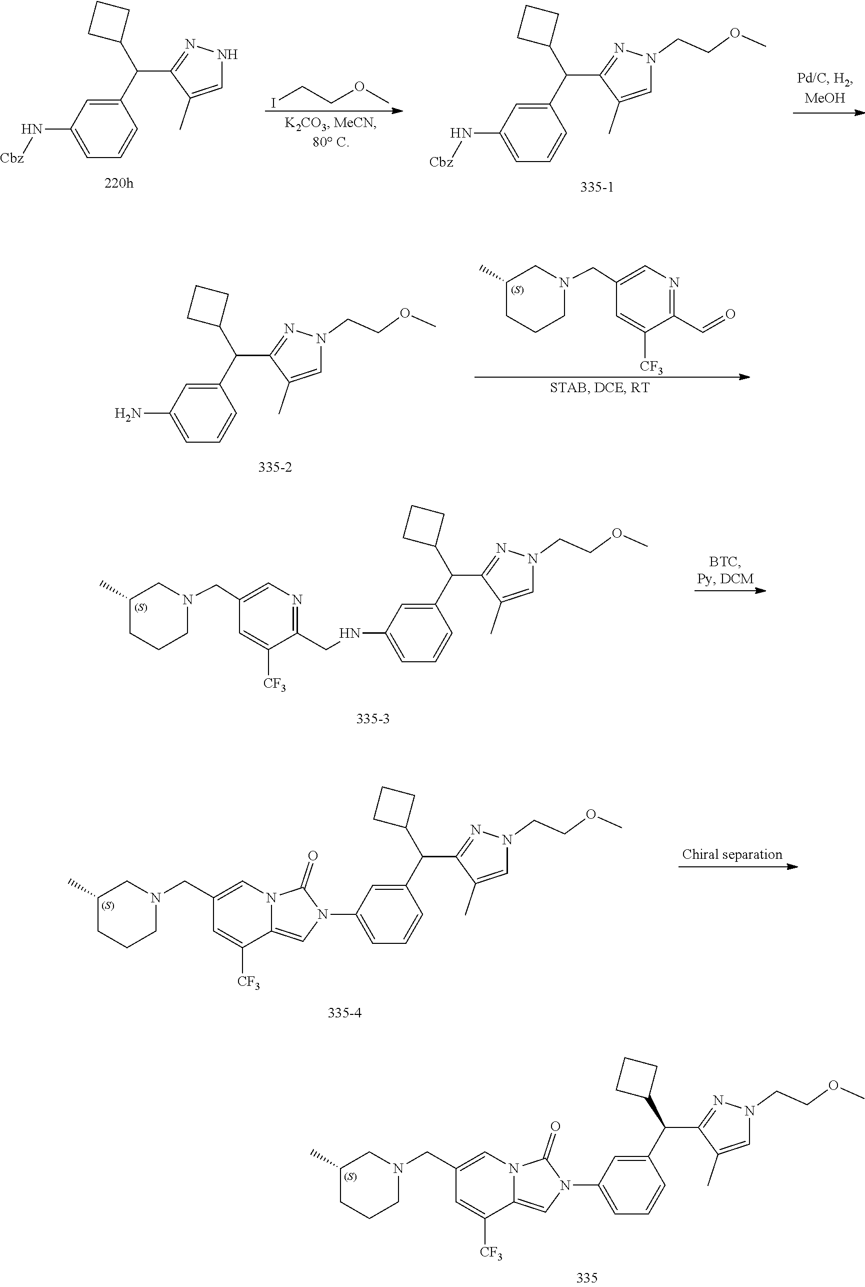

### 1. Synthesis of 335-1

To a stirred solution of 220 h (3.2 g, 8,523 mmol, 1 equiv) and 1-iodo-2-methoxyethane (3.17 g, 17.046 mmol, 2 equiv) in MeCN (50 mL) was added K2CO3 (3.53 g, 25.569 mmol, 3 equiv) at room temperature. The resulting mixture was stirred overnight at 80° C. The resulting mixture was dilated with water (150 mL). The aqueous layer was extracted with EtOAc (2×80 mL). The resulting mixture was concentrated under vacuum. The residue was purified by Prep-TLC (DCM/MeOH 40:1) to afford 335-1 (750 mg, 18.88%) as a light yellow solid.

### 2. Synthesis of 335-2

To a solution of 335-1 (750 mg, 1.730 mmol, 1 equiv) in MeOH (20 mL) was added Pd/C (100 mg) under nitrogen atmosphere in a 100 mL round-bottom flask. The mixture was hydrogenated at room temperature for 4 h under hydrogen atmosphere using a hydrogen balloon, filtered through a Celite pad and concentrated under reduced pressure. The crude product was used in the next step directly without further purification. This resulted in 335-2 (500 mg, 86.88%) as an off-white solid,

### 3. Synthesis of 335-3

To a stirred solution of 335-2 (500 mg, 1,670 mmol, 1 equiv) and 1-2 (573.71 mg, 2.004 mmol, 1.2 equiv) in DCE (10 mL) was added STAB (707.84 rug, 3.340 mmol, 2 equiv) at room temperature. The resulting mixture was stirred overnight at room temperature. The reaction was quenched with saturated NaHCO3 (aq.) (50 mL) at room temperature. The aqueous layer was extracted with DCM (2×50 mL). The resulting mixture was concentrated under vacuum. The residue was purified by Prep-TLC (DCM/MeOH 40:1) to afford 335-3 (720 mg, 70.38%) as a light yellow oil.

### 4. Synthesis of 335-4

To a stirred solution of 335-3 (700 mg, 1.229 mmol, 1 equiv) and pyridine (971.91 mg 12.290 mmol, 10 equiv) in DCM (10 mL) was added Triphosgene (182.29 mg, 0.615 mmol, 0.5 equiv) at 0° C. under nitrogen atmosphere. The resulting mixture was stirred for 10 min at room temperature. The reaction was quenched with saturated NaHCO₃ (aq.) (50 mL) at room temperature. The aqueous layer was extracted with DCM (2×20 mL). The resulting mixture was concentrated under vacuum. The residue was purified by Prep-TLC (DCM/MeOH 30:1) to afford 335-4 (550 mg, 72.14%) as a yellow solid.

### 5. Synthesis of 335

The 335-4 (400 mg) was purified by Prep-Chiral-HPLC with the following conditions (Column: CHIRALPAK IF, 2*25 cm, 5 μm; Mobile Phase A: Hex (0.5% 2M NH3-MeOH), Mobile Phase B: IPA; Flow rate: 20 mL/min; Gradient: 10% B to 10% B in 15 min; Wave Length: 220/254 nm; RT1 (min): 9.008; RT2 (min): 11.926; the second peak is product) to afford 335 (125.6 mg, 30.05%) as a yellow solid.

LC-MS: (ES, m/z): [M+H]+ 596. H-NMR: (400 MHz, DMSO-d6, δ ppm): 0.82-0.93 (m, 4H), 1.44-1.52 (m, 1H), 1.52-1.72 (m, 5H), 1.72-1.84 (m, 4H), 1.84 (s, 3H), 1.84-2.00 (m, 2H), 2.71-2.85 (m, 2H), 3.09-3.17 (m, 1H), 3.19 (s, 3H), 3.25 (s, 2H), 3.62-3.64 (m, 2H), 3.93-3.96 (d, 1H), 4.11-4.14 (m, 2H), 7.00 (s, 1H), 7.22-7.25 (m, 2H), 7.31 (s, 1H), 7.35-7.39 (m, 1H), 7.58-7.60 (d, 1H), 7.60 (s, 1H), 7.66 (s, 1H).

### Example 336. Synthesis of Compound 336

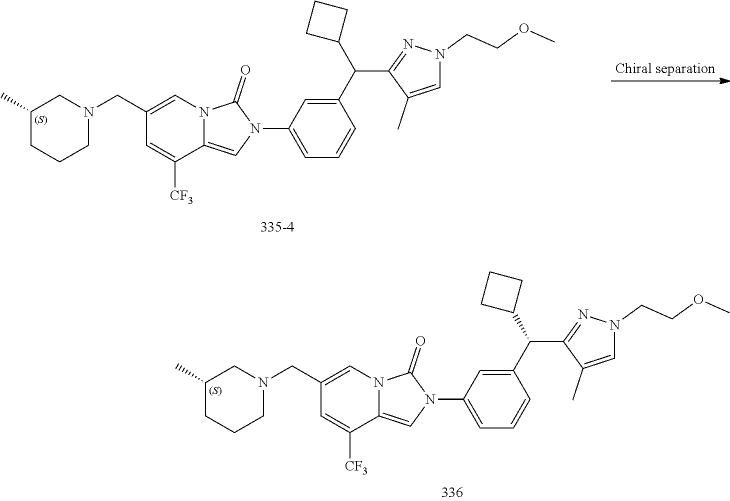

### 1. Synthesis of 336

The 335-4 (400 mg) was purified by Prep-Chiral-HPLC with the following conditions (Column: CHIRALPAK IF, 2*25 cm, 5 μm; Mobile Phase A: Hex (0.5% 2M NH3-MeOH), Mobile Phase B: IPA; Flow rate: 20 mL/min; Gradient: 10% B to 10% B in 15 min; Wave Length: 220/254 nm; RT1 (min): 9.008; RT2 (min): 11.926; the first peak is product) to afford 336 (117.8 mg, 28.33%) as a yellow solid.

LC-MS: (ES, m/z): [M+H]+ 596. H-NMR: (400 MHz, DMSO-d6, δ ppm): 0.81-0.93 (m, 4H), 1.43-1.52 (m, 1H), 1.52-1.71 (m, 5H), 1.71-1.84 (m, 4H), 1.84 (s, 3H), 1.84-2.00 (m, 2H), 2.71-2.85 (m, 2H), 3.09-3.18 (m, 1H), 3.19 (s, 3H), 3.26 (s, 2H), 3.62-3.64 (m, 2H), 3.93-3.95 (d, 1H), 4.11-4.14 (m, 2H), 7.00 (s, 1H), 7.22-7.25 (m, 2H), 7.31 (s, 1H), 7.35-7.39 (m, 1H), 7.58-7.60 (d, 1H), 7.60 (s, 1H), 7.66 (s, 1H).

### Example 337. Synthesis of Compound 337

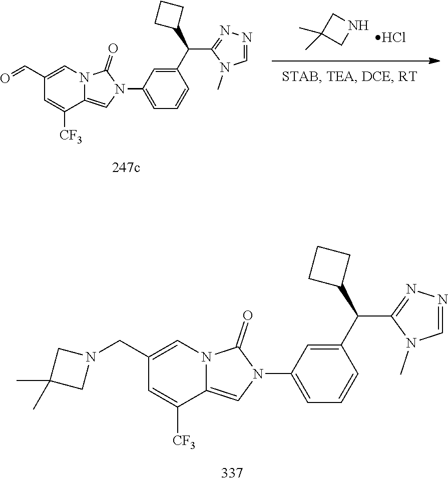

### Synthesis of 337

To a stirred solution of 247c (300 mg, 0.659 mmol, 1 equiv) and (3-methylazetidin-3-yl)methylium hydrochloride (158.88 mg, 1.318 mmol, 2 equiv) in DCE (5 ML) was added TEA (133.31 mg, 1318 mmol, 2 equiv) at room temperature. The resulting mixture was stirred for 1 h at room temperature. To the above mixture was added STAB (279.21 mg, 1.317 mmol, 2.00 equiv) at room temperature. The resulting mixture was stirred for additional 3 h at room temperature. The reaction was quenched with saturated NH₄Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-HPLC with the following conditions (Column: XBridge Prep OBD C1 8 Column, 30*150 mm, 5 μm; Mobile Phase A: Water (1O mmol/L NH₄CO₃), Mobile Phase B: ACN; Flow rate: 60 mL/min; Gradient: 34% B to 64% B in 8 min, Wave Length: 220 nm; RT1 (min): 7.53) to afford 337 (124.9 mg, 36.00%) as a yellow solid.

LC-MS: (ES, m/z): [M+H]⁺525. H-NMR: (400 MHz, DMSO-d6, δ ppm): 1.18 (s, 6H), 1.67-1.87 (m, 5H), 2.07-2.12 (m, 1H), 2.91 (s, 6H), 3.18-3.25 (m, 1H), 3.35 (s, 2H), 3.42 (s, 3H), 4.24-4.27 (d, 1H), 6.97 (s, 1H), 7.18-7.20 (d, 1H), 7.31 (s, 1H), 7.42-7.45 (m, 1H), 7.62 (s, 1H), 7.68-7.70 (d, 1H), 7.74 (s, 1H), 8.33 (s, 1H).

### Example 338. Synthesis of Compound 338

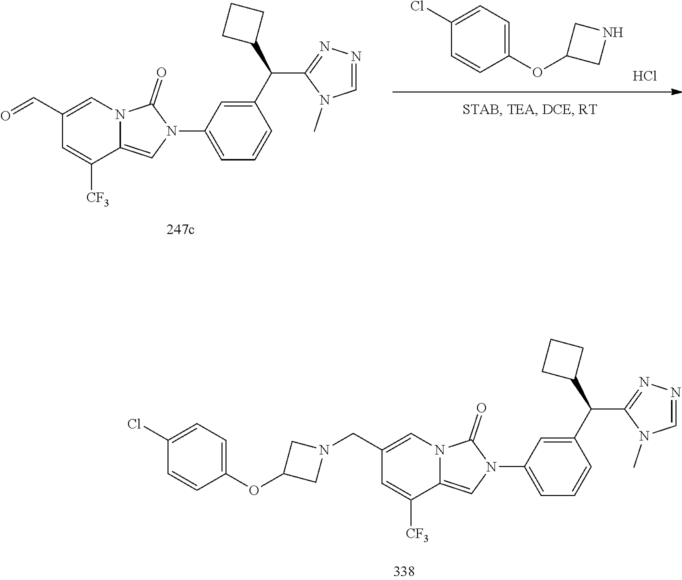 [SMILES: Cn1cnnc1[C@@H](c1cccc(-n2cc3c(C(F)(F)F)cc(C=O)cn3c2=O)c1)C1CCC1 ; Clc1ccc(OC2CNC2)cc1 ; Cl ; Cn1cnnc1[C@@H](c1cccc(-n2cc3c(C(F)(F)F)cc(CN4CC(Oc5ccc(Cl)cc5)C4)cn3c2=O)c1)C1CCC1]

### 1. Synthesis of 338

To a stirred solution of 247c (300 mg, 0.659 mmol, 1 equiv) and 3-(4-chlorophenoxy)azetidine hydrochloride (9.66 mg, 0.044 mmol, 2 equiv) in DCE (5 mL) was added TEA (133.31 mg, 1.318 mmol, 2 equiv) at room temperature. The resulting mixture was stirred for 1 h at room temperature. To the above mixture was added STAB (279.21 mg, 1,317 mmol, 2.00 equiv) at room temperature. The resulting mixture was stirred 3 h at room temperature. The reaction was quenched with saturated NH4Cl (aq.) (50 m L) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by HP-Flash with the following conditions (Column: Welch Utimate AQ-C18, 50*250 mm*10 μm; Mobile Phase A: Water (10 mmol/L NH4HCO3), Mobile Phase B: ACN; Flow rate: 100 mL/min; Gradient: 45% B to 75% B in 20 min, Wave Length: 220 nm; RT (min): 20) to afford 338 (1253 mg, 30.53%) as a yellow solid.

LC-MS: (ES, m/z): [M+H]+ 623. H-NMR: (400 MHz, DMSO-d6, δ ppm): 1.67-1.87 (m, 5H), 2.07-2.12 (m, 1H), 3.06-3.10 (m, 2H), 3.20-3.25 (m, 1H), 3.42 (s, 3H), 3.45 (s, 2H), 4.24-4.27 (d, 1H), 4.81-4.84 (m, 1H), 6.85-6.88 (d, 2H), 6.99 (s, 1H), 7.18-7.20 (d, 1H), 7.29-7.33 (m, 3H), 7.42-7.46 (m, 1H), 7.62 (s, 1H), 7.67-7.73 (m, 3H), 8.33 (s, 1H).

### Example 339. Synthesis of Compound 339_P1 and 339_P2

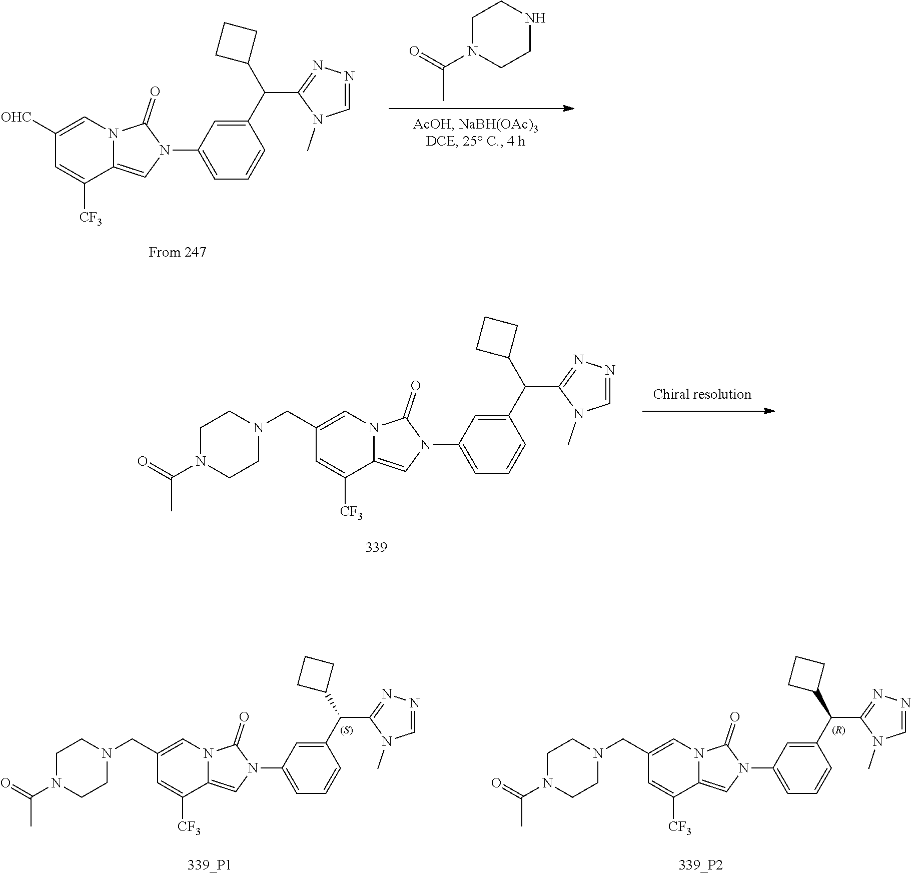

### 1. Synthesis of 339

To a solution of 2-[3-[cyclobutyl-(4-methyl-1,2,4-triazol-3-yl)methyl]phenyl]-3-oxo-8-(trifluoromethyl) imidazo[1,5-a]pyridine-6-carbaldehyde (200 mg, 439.14 umol, 1.0 equiv) and 1-piperazin-1-ylethanone (168 mg, 1.32 mmol, 3.0 equiv) in DCE (10 mL) was added AcOH (26 mg, 439 mmol, 1.0 equiv) at 25° C. After addition, the mixture was stirred at this temperature for 2 hr, and then NaBH(OAc)3 (186 mg, 878 umol, 2.0 equiv) was added. The resulting mixture was stirred at 25° C. for 2 hr. The reaction was poured into water (10 mL) and the resulting mixture was extracted with CH2Cl2 (2×10 mL). The organic phase was washed with brine (10 mL), dried over anhydrous Na2SO4. After filtration, the filtrate concentrated in vacuum to give a residue. The residue was purified by prep-HPLC (column: Phenomenex C18 75*30 mm*3 um; Mobile Phase A: Water (NH4HCO3), Mobile Phase B: ACN; Flow rate: 50 mL/min; Gradient: 20% B to 50% B in 8 min; Wave Length: 220 nm; RT1 (main): 5.1) to afford 339 (35 mg, 13% yield) as a yellow solid.

### 2. Synthesis of 339_P1& P2

The 339 (35 mg) was purified by Chiral separation with the following conditions (column: DAICEL CHIRALCEL OD (250 mm*30 mm, 10 um); Mobile Phase: [Neu-MeOH]; B %: 20%-45%, 15 min, Flow rate: 70 mL/min; Wave Length: 220/254 nm; RT1 (min): 1.29: RT2 (min): 1.44) to afford 339 P1 (11.2 mg, 62% yield) as a yellow solid.

MS-339_P1 (ES, m/z): [M+H]+ 568.2.

H-NMR-339_P1: (400 MHz, DMSO-d6) δ 8.34 (s, 1H), 7.70-7.74 (m, 3H), 7.43-7.47 (m, 1H), 7.33 (s, 1H), 7.20-7.22 (m, 1H), 7.04 (s, 1H), 4.27 (d, J=5.6 Hz, 1H), 3.37-3.44 (m, 7 H), 3.27-3.29 (m, 2H), 3.24-3.25 (m, 1H), 2.51-2.52 (m, 1H), 2.35-2.39 (m, 4H), 2.09-2.13 (m, 1H), 1.99 (s, 3H), 1.78-1.81 (m, 5H).

339_P2-0 (11.1 mg, 62% yield) as a yellow solid.

MS-339-P2: (ES, m/z): [M+H]⁺ 568.2.

H-NMR-339-P2: (400 MHz, DMSO-d6) δ 8.34 (s, 1H), 7.70-7.74 (m, 3H), 7.43-7.47 (m, 1H), 7.33 (s, 1H), 7.20-7.22 (m, 1H), 7.04 (s, 1H), 4.27 (d, J=5.6 Hz, 1H), 3.37-3.44 (m, 7H), 3.27-3.29 (m, 2H), 3.24-3.25 (m, 1H), 2.51-2.52 (m, 1H), 2.35-2.39 (m, 4H), 2.09-2.13 (m, 1H), 1.99 (s, 3H), 1.78-1.81 (m, 5H).

### Example 340. Synthesis of Compound 340_P1 and 340_P2

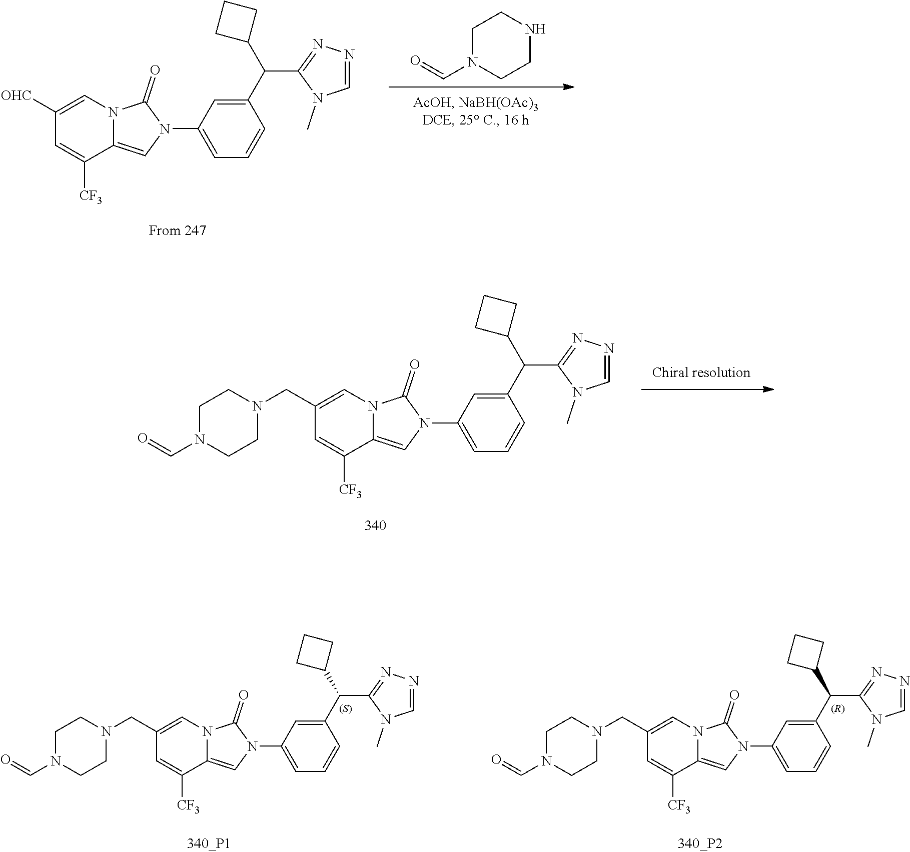

### 1. Synthesis of 340

To a solution of piperazine-1-carbaldehyde (150 mg, 1.32 mmol, 3.0 equiv), 2-[3-[cyclobutyl-(4-methyl-1,2,4-triazol-3-yl)methyl]phenyl]-3-oxo-8-(trifluoromethyl)imidazo[1,5-a]pyridine-6-carbaldehyde (200 mg, 439 mmol, 1.0 equiv) in DCE (10 mL) was added AcOH (26 mg 439 mmol, 1.0 equiv) at 25° C. After addition, the mixture was stirred at this temperature for 2 hrs, and then NaBH(OAc)3 (186 mg, 878 umol, 2.0 equiv) was added. The resulting mixture was stirred at 25° C. for 14 hrs. The reaction was poured into water (10 mL) and the resulting mixture was extracted with DCM (2×10 mL). The organic phase was washed with brine (10 mL), dried over anhydrous Na2SO4. After filtration, the filtrate was concentrated in vacuum to give a residue. The residue was purified by prep-HPLC (column: Phenomenex C18 75*30 mm*3 um; Mobile Phase A: Water (NH4HCO3), Mobile Phase B: ACN; Flow rate: 50 mL/min; Gradient: 25% B to 55% B in 8 min; Wave Length: 220 nm; RT1 (min): 5.1) to afford 340 (30 mg, 11%) as a light yellow solid.

### 2. Synthesis of 340_P1& P2

The 340 (30 mg) was purified by Chiral separation with the following conditions (column: DAICEL CHIRALCEL OD (250 mm*30 mm, 10 um); Mobile Phase: [Neu-MeOH]; B %: 35%-35%, 13 min, Flow rate: 70 mL/min; Wave Length: 220/254 nm; RT1 (min): 1.37; RT2 (min): 1.44) to afford 340_P1 (5.4 mg, 35% yield) as a yellow solid.

MS-340-P1: (ES, m/z): [M+H]+ 554.2.

H-NMR-340-P1: (400 MHz, DMSO-d6) δ 8.32 (s, 1H), 7.99 (s, 1H), 7.71-7.72 (m, 1H), 7.69-7.70 (m, 2H), 7.43-7.45 (m, 1H), 7.41 (s, 1H), 7.20-7.21 (m, 1H), 7.03 (s, 1H), 4.25 (d, J=5.2 Hz, 1H), 3.42 (s, 3H), 3.38-3.39 (m, 4H), 3.27-3.29 (m, 2H), 3.24-3.25 (m, 1H), 2.41-2.42 (m, 2H), 2.35-2.39 (m, 2H), 2.09-2.13 (m, 1H), 1.76-1.79 (m, 4H), 1.69-1.70 (m, 1H).

340_P2 (4.8 mg, 30% yield) as a yellow solid.

MS-340-P2: (ES, m/z): [M+H]+ 554.2.

H-NMR-340-P2: (400 MHz, DMSO-d6) δ 8.32 (s, 1H), 7.99 (s, 1H), 7.71-7.72 (m, 1H), 7.69-7.70 (m, 2H), 7.43-7.45 (m, 1H), 7.41 (s, 1H), 7.20-7.21 (m, 1H), 7.03 (s, 1H), 4.25 (d, J=5.2 Hz, 1H), 3.42 (s, 3H), 3.38-3.39 (m, 4H), 3.27-3.29 (m, 2H), 3.24-3.25 (m, 1H), 2.41-2.42 (m, 2H), 2.35-2.39 (m, 2H), 2.09-2.13 (m, 1H), 1.76-1.79 (m, 4H), 1.69-1.70 (m, 1H).

### Example 341. Synthesis of Compound 341

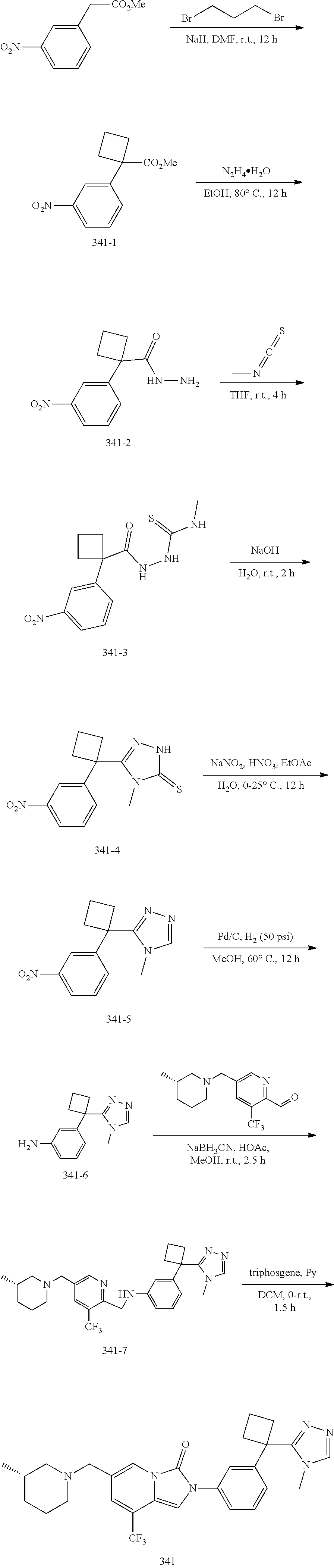

### 1. Synthesis of 341-1

To a solution of methyl 2-(3-nitrophenyl)acetate (10 g, 51.2 mmol, 1 equiv) and 1,3-dibromopropane (5.22 mL, 51.24 mmol, 1 equiv) in DMF (300 mL) was added NaH (4.1 g, 102 mmol, 60% purity, 2 equiv) at room temperature. Then the mixture was stirred at room temperature for 12 hr. The reaction mixture was diluted with water (150 mL), extracted with EtOAc (150 mL×3). The combined organic layers were dried over anhydrous Na2SO4, filtered and the filtrate was concentrated in vacuum. The residue was purified by normal phase SiO₂ chromatography (0-5% EtOAc/PE) to afford 341-1 (5 g, 41.48%) as a white solid.

H-NMR-341-1: (400 MHz, CHLOROFORM-d) δ 8.14 (t, J=2.0 Hz, 1H), 8.11-8.05 (m, 1H), 7.59 (d, J=8.0 Hz, 1H), 7.52-7.44 (m, 1H), 3.64 (s, 3H), 2.94-2.81 (m, 2H), 2.57-2.44 (m, 2H), 2.18-2.04 (m, 1H), 1.97-1.82 (m, 1H)

### 2. Synthesis of 341-2

To a solution of 341-1 (4.93 g, 20.9 mmol, 1 equiv) in EtOH (50 mL) was added N2H4·H2O (15.6 mL, 314.37 mmol, 98% purity, 15 equiv) at room temperature. The mixture was heated to 80° C. and stirred for 12 hr. The reaction mixture was cooled to room temperature and concentrated in vacuum to remove EtOH (40 mL), then the mixture was diluted with water (50 mL), and extracted with CH2Cl2 (40 mL×3). The combined organic layers were dried over anhydrous Na2SO4, filtered and the filtrate was concentrated in vacuum to afford 341-2 (4.3 g, 87.22%) as a white solid, which was used directly without further purification.

### 3. Synthesis of 341-3

To a solution of 341-2 (4.3 g, 18.3 mmol, 1 equiv) in THF (50 m L) was added methylimino(thioxo)methane (2.67 g, 36.5 mmol, 2 equiv) at room temperature. The mixture was stirred at room temperature for 4 hr. The reaction mixture was diluted water (50 mL), extracted with EtOAc (50 mL×3). The combined organic layers were dried over anhydrous Na2SO4, filtered and concentrated in vacuum to afford 341-3 (4.3 g, crude) as a white solid, which was used directly in the next step.

### 4. Synthesis of 341-4

To a solution of NaOH (4.46 g, 111 mmol, 8 equiv) in water (45 mL) was added 341-3 (4.3 g, 13.9 mmol, 1 equiv). The mixture was stirred at 25° C. for 2 hr. The reaction mixture was diluted with water (50 mL), and adjusted pH to 3 by 1N HC. Then the mixture was filtered and the filter cake was concentrated in vacuum to afford 341-4 (4 g, 98.8%) as a white solid.

H-NMR-341-4: (400 MHz, DMSO-d6) δ 13.84 (s, 1H), 8.21-8.15 (m, 1H), 8.06 (t, J=2.0 Hz, 1H), 7.80-75 (m, 1H), 7.74-7.69 (m, 1H), 3.01 (s, 3H), 2.94-2.85 (m, 2H), 2.73-2.65 (m, 2H), 2.09-1.97 (m, 2H)

### 5. Synthesis of 341-5

HNO3 (10.03 mL, 151.55 mmol, 68% purity, 10 equiv) was added to water (140 mL) to afford the diluted HNO3 solution (1 M, 150 mL). To a solution of 341-4 (4.4 g, 15.15 mmol, 1 eq) and NaNO2 (10.46 g, 151.55 mmol, 10 equiv) in water (44 mL) and EtOAc (4.40 mL, 44.94 mmol, 2.97 equiv) was added diluted HNO3 solution (1 M, 150 mL) dropwise at 0° C. Then the mixture was stirred at 25° C. for 12 hr. The reaction was quenched by the addition of Sat.NaHCO₃ (300 mL) The aqueous layer was extracted with CH2Cl2 (150 mL×3). The combined organic layers were dried over anhydrous Na2SO4, filtered and the filtrate was concentrated in vacuum to afford 341-5 (3.8 g, crude) as a yellow solid.

### 6. Synthesis of 341-6

To a solution of 341-5 (1 g, 3.87 mmol, 1 equiv) in MeOH (20 ml) was added Pd/C (1 g, 10% purity). The mixture was degassed and purged with 112 for 3 times, and then the mixture was stirred at 60° C. for 12 hr under H2 (50 psi). The reaction mixture was filtered through celite, and the filtrate was concentrated in vacuum o afford 341-6 (0.78 g, 88.24%) as a gray solid.

H-NMR-341-6: (400 MHz, DMSO-d6) δ 8.32 (s, 1H), 6.99 (t, J=8.0 Hz, 1H), 6.44-6.39 (m, 2H), 6.38-6.36 (m, 1H), 5.06 (s, 2H), 3.17 (s, 3H), 2.88-2.77 (m, 2H), 2.58-2.52 (m, 2H), 1.99-1.88 (m, 2H)

### 7. Synthesis of 341-7

To a solution of 341-6 (0.68 g, 2.98 mmol, 1 equiv) and I-2 (767 mg, 2.68 mmol, 0.9 equiv) in MeOH (15 mL) was added AcOH (511 ul, 8.94 mmol, 3 equiv). The mixture was stirred at 25° C. for 1 hr under nitrogen atmosphere, then NaBH3CN (374 mg, 5.96 mmol, 2 equiv) was added to the mixture. The resulted mixture was stirred at 25° C. for 1.5 hr under nitrogen atmosphere. The reaction mixture was concentrated in vacuum to remove MeOH, the residue was diluted with Sat. NaHCO3 (20 mL), extracted with EtOAc (20 mL×3). The combined organic layers were dried over anhydrous Na2SO4, filtered and the filtrate was concentrated in vacuum. The residue was purified by normal phase SiO₂ chromatography (0-10% MeOH CH2Cl2) to afford 341-7 (1.53 g, 89.5%) as a yellow oil.

H-NMR-341-7: (400 MHz, CHLOROFORM-d) δ 8.58 (s, 1H), 7.94-7.91 (m, 1H), 7.88 (s, 1H), 7.16-7.10 (m, 1H), 6.60 (d, J=7.6 Hz, 1H), 6.57-6.53 (m, 2H), 5.35 (s, 1H), 5.23 (s, 2H), 4.46 (s, 2H), 3.11 (s, 3H), 2.92-2.87 (m, 2H), 2.66-2.59 (m, 3H), 2.07-2.00 (m, 2H), 1.92-1.81 (m, 1H), 1.64-1.55 (m, 6H), 0.84-0.75 (m, 4H)

### 8. Synthesis of 341

To a solution of 341-7 (0.7 g, 1.40 mmol, 1 equiv) in DCM (10 mL) was added pyridine (680 uL, 8.42 mmol, 6 equiv) and bis(trichloromethyl) carbonate (208 mg, 702 umol, 0.5 equiv) at 0° C. under nitrogen atmosphere, the mixture was stirred at 0° C. for 1.5 hr under nitrogen atmosphere. The reaction mixture was diluted with Sat. NaHCO₃ (20 mL), extracted with CH2Cl2 (15 mL×3). The combined organic layers were dried over anhydrous Na2SO4, filtered and the filtrate was concentrated in vacuum. The residue was purified by normal phase SiO2 chromatography (0-10% MeOH CH2Cl2) to give crude product. The crude product (450 mg) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 15% B to 55% B in 8 min; Wave Length: 220 am; RT1 (min): 5.5) to afford 341 (110 mg, 14.79%) as a yellow solid.

LCMS-341 (ES, m/z): [M+H]+ 525.2. H-NMR-341 (400 MHz, METHANOL-d4) δ 8.47 (s, 1H), 8.38 (s, 1H), 7.83 (s, 1H), 7.76 (t, J=2.0 Hz, 1H), 7.64-7.53 (m, 2H), 7.38-7.32 (m, 1H), 7.20 (s, 1H), 7.14 (s, 1H), 3.73 (d, J=12.4 Hz, 2H), 3.35 (s, 3H), 3.26-3.11 (m, 2H), 3.08-2.98 (m, 2H), 2.89-2.78 (m, 2H), 2.48-2.34 (m, 1H), 2.21-2.07 (m, 3H), 1.82 (s, 3H), 1.77-1.65 (m, 1H), 1.14-1.02 (m, 1H), 0.96 (d, J=6.4 Hz, 3H).

### Example 342. Synthesis of Compound 342

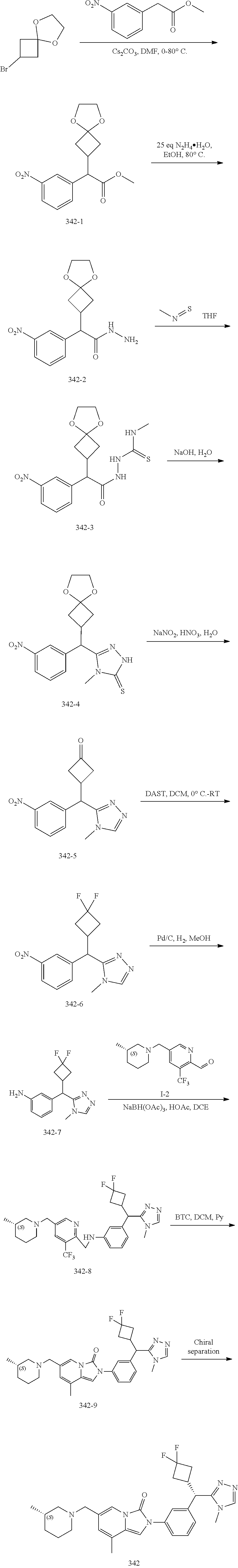

### 1. Synthesis of 342-1

Into a 250 mL 3-necked round-bottom flask were added methyl 2-(3-nitrophenyl)acetate (4 g, 20.495 mmol, 1 equiv), DMF (50 mL), Cs2CO3 (33.39 g, 102.475 mmol, 5 equiv) and 2-bromo-5,8-dioxaspiro[3.4]octane (8.3 g, 42996 mmol, 2.10 equiv) at 0° C. The resulting mixture was stirred for overnight at 80° C. under nitrogen atmosphere. The reaction was quenched by the addition of sat. NH4Cl (aq.) (200 mL) at room temperature. The aqueous layer was extracted with EtOAc (3×100 mL). The resulting mixture was concentrated under reduced pressure. The residue was purified by silica gel column chromatography, eluted with PE/EA (20:1) to afford 342-1 (1.8 g, 28.58%) as a light yellow oil.

### 2. Synthesis of 342-2

Into a 100 mL round-bottom flask were added 342-1 (1.8 g, 5.857 mmol, 1 equiv), EtOH (22 mL) and Hydrazine (4.69 g, 146.425 mmol, 25 equiv) at room temperature. The resulting mixture was stirred for overnight at 80° C. The reaction was quenched by the addition of Water (100 mL) at room temperature. The aqueous layer was extracted with EtOAc (3×50 mL). The resulting mixture was concentrated under reduced pressure to afford 342-2 (1.7 g, 94.44%) as an off-white solid.

### 3. Synthesis of 342-3

Into a 100 mL round-bottom flask were added 342-2 (1.8 g, 5.857 mmol, 1 equiv), tetrahydrofuran (22 mL) and methyl isothiocyanate (1.07 g, 14.643 mmol, 2.5 equiv) at room temperature. The resulting mixture was stirred for overnight at room temperature. The reaction was quenched by the addition of Water (40 mL) at room temperature. The resulting mixture was filtered; the filter cake was washed with water (3×20 mL). The filtrate was concentrated under reduced pressure to afford 342-3 (1.8 g, 80.78%) as an off-white solid.

### 4. Synthesis of 342-4

Into a 100 mL round-bottom flask were added NaOH (10 ml, 1M), and 342-3 (1 g, 2.629 mmol, 1 equiv) at room temperature. The resulting mixture was stirred for overnight at room temperature. The mixture/residue was acidified to pH 6 with 1M HCl (aq.). The resulting mixture was filtered; the filter cake was washed with water (3×4 mL). The filtrate was concentrated under reduced pressure to afford 342-4 (0.9 g, 94.48%) as an off-white solid.

### 5. Synthesis of 342-5

To a stirred solution of 342-4 (1.8 g, 4.967 mmol, 1 equiv) in H2O (8 ml) was added NaNO2 (3.43 g, 49.670 mmol, 10 equiv) at room temperature. To the above mixture was added HNO3 (52 ml, 1M) dropwise at 0° C. The resulting mixture was stirred for additional 1 h at 0° C. The mixture was neutralized to pH 7 with saturated NaHCO₃ (aq.). The aqueous layer was extracted with EtOAc (3×10 mL). The resulting mixture was concentrated under reduced pressure. The residue was purified by silica gel column chromatography, eluted with PE/EA (10:1) to afford 342-5 (1 g, 70.32%) as an off-white solid.

### 6. Synthesis of 342-6

Into a 100 mL round-bottom flask were added 342-5 (1 g, 3.493 mmol, 1 equiv), DCM (15 mL) and DAST (2.25 g, 13.972 mmol, 4 equiv) at 0° C. The resulting mixture was stirred for overnight at room temperature under nitrogen atmosphere. The mixture was neutralized to pH 7 with saturated NaHCO₃ (aq.). The aqueous layer was extracted with EtOAc (3×15 mL). The resulting mixture was concentrated under reduced pressure. The residue was purified by silica gel column chromatography, eluted with PE/EA (8:1) to afford 342-6 (150 mg, 13.93%) as a light yellow solid.

### 7. Synthesis of 342-7

To a solution of 342-6 (150 mg, 0.487 mmol, 100 equiv) in 5 mL MeOH was added Pd/C (10%, 51 mg) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure to afford 342-7 (110 mg, 81.23%) as a light yellow solid.

### 8. Synthesis of 342-8

Into a 100 mL round-bottom flask were added 342-7 (110 mg, 0.395 mmol, 1 equiv), DCE (3 mL), 5-{[(3S)-3-methylpiperidin-1-yl]methyl}-3-(trifluoromethyl)pyridine-2-carbaldehyde (113.16 mg, 0,395 mmol, 1 equiv), HOAc (23.74 mg, 0,395 mmol, 1 equiv) and NaBH(OAc)3 (167.54 mg, 0,790 mmol, 2 equiv) at room temperature. The resulting mixture was stirred for overnight at room temperature. The reaction was quenched by the addition of sat. NH4Cl (aq.) (15 mL) at room temperature. The aqueous layer was extracted with EtOAc (3×10 mL). The resulting mixture was concentrated under reduced pressure. The residue was purified by Prep-TLC, eluted with CH2Cl2/MeOH (12:1) to afford 342-8 (90 mg, 41.51%) as a light yellow solid.

### 9. Synthesis of 342-9

Into a 50 mL round-bottom flask were added 342-8 (90 mg, 0,164 mmol, 1 equiv), Pyridine (77.86 mg, 0.984 mmol, 6 equiv) and d Triphosgene (19.47 mg, 0.066 mmol, 0.4 equiv) at 0° C. The resulting mixture was stirred for 15 min at 0° C. under nitrogen atmosphere. The reaction was quenched by the addition of sat. NaHCO₃ (aq.) (10 ML) at room temperature. The aqueous layer was extracted with CH2Cl2 (3×10 mL). The resulting mixture was concentrated under reduced pressure. The residue was purified by silica gel column chromatography, elated with CH2Cl2/MeOH (10:1) to afford 342-9 (60 mg, 63.65%) as a light yellow solid.

### 10. Synthesis of 342

The 342-9 (60 mg) was purified by Chiral separation with the following conditions (Column: CHIRALPAK IC, 2*25 cm, 5 μm; Mobile Phase A: Hex (0.5% 2M NH3-MeOH)-HPLC, Mobile Phase B: MeOH: EtOH=1:1-HPLC; Flow rate: 20 mL/min; Gradient: 30% B to 30% B in 16 min; Wave Length: 220/254 nm; RT1 (min): 10.644; RT2 (min): 16.675; The first peak was the product. Sample Solvent: MeOH:DCM=1:1; Injection Volume: 4.4 mL; Number of Runs: 2) to afford 342 (23.8 mg, 39.67%) as a light yellow solid.

H-NMR: 1H NMR (300 MHz, CD3OD-d4) δ 0.90-0.96 (m, 4H), 1.30-1.34 (m, 1H), 1.61-1.78 (m, 4H), 1.99-2.04 (m, 1H), 2.33-2.54 (m, 3H), 2.84-2.93 (m, 3H), 3.15-3.17 (m, 1H), 3.32-3.37 (m, 2H), 3.52 (s, 3H), 4.33-4.36 (d, 1H), 6.69 (s, 1H), 7.13-7.16 (m, 2H), 7.28-7.30 (m, 1H), 7.52-7.56 (m, 1H), 7.68-7.73 (m, 3H), 8.45 (s, 1H).

### Example 343. Synthesis of Compound 343

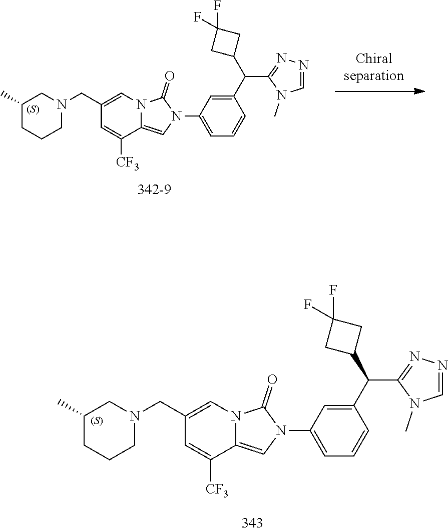

### 1. Synthesis of 343

The 342-9 (60 mg) was purified by Chiral separation with the following conditions (Column: CHIRALPAK IC, 2*25 cm, 5 μm; Mobile Phase A: Hex (0.5% 2M NH3-MeOH)-HPLC, Mobile Phase B: MeOH: EtOH=1:1-HPLC; Flow rate: 20 mL/min; Gradient: 30% B to 30% B in 16 min; Wave Length: 220/254 nm; RT1 (min): 10.644; RT2 (min): 16,675; The second peak was the product. Sample Solvent: MeOH:DCM=1:1; Injection Volume: 4.4 mL; Number of Runs: 2) to afford 343 (24.3 mg, 40.50%) as a light yellow solid.

H-NMR: 1H NMR (300 MHz, CD3OD-d4) S 0.90-0.96 (m, 4H), 1.62-1.81 (m, 5H), 1.91-2.04 (m, 1H), 2.33-2.48 (m, 1H), 2.50-2.54 (m, 2H), 2.84-2.93 (m, 3H), 3.15-3.17 (m, 1H), 3.32-3.37 (m, 2H), 3.52 (s, 3H), 4.33-4.36 (d, 1H), 6.69 (s, 1H), 7.13-7.16 (m, 2H), 7.28-7.30 (m, 1H), 7.52-7.56 (m, 1H), 7.68-7.73 (m, 3H), 8.45 (s, 1H).

### Example 344. Synthesis of Compound 344

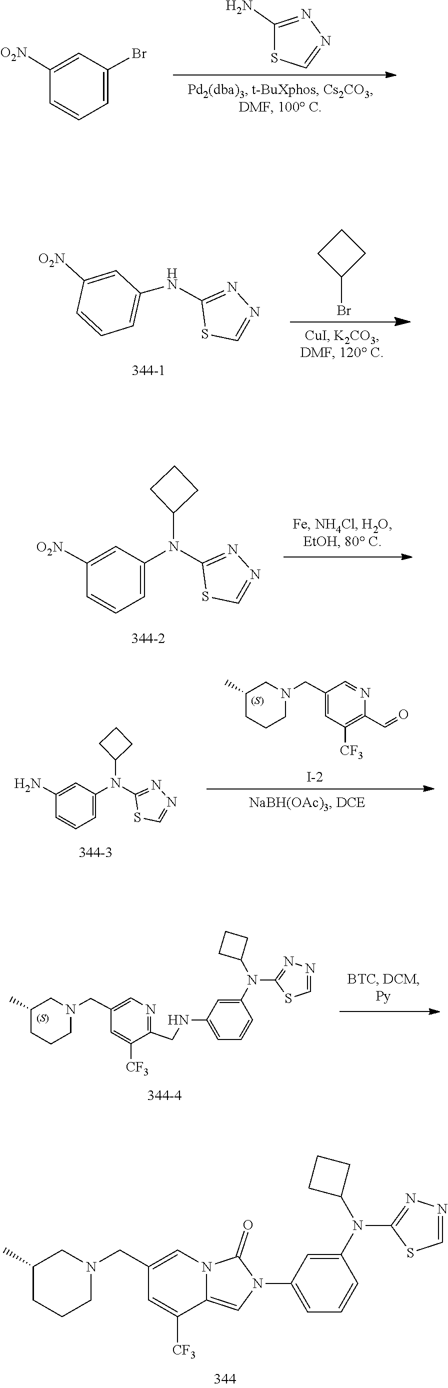

### 1. Synthesis of 344-1

Into a 500 mL 3-necked round-bottom flask were added 3-bromo-1-nitrobenzene (10 g, 49,503 mmol, 1 equiv), 2-amino-1,3,4-thiadiazole (5.01 g, 49,503 mmol, 1 equiv), Pd2(dba)3 (4.53 g, 4.950 mmol, 0.1 equiv), t-BuXPhos (4.20 g, 9.901 mmol, 0.2 equiv), Cs2CO3 (32.26 g, 99,006 mmol, 2 equiv) and DMF (300 mL) at room temperature. The resulting mixture was stirred for overnight at 100° C. under nitrogen atmosphere. The resulting mixture was diluted with water (500 mL). The aqueous layer was extracted with EtOAc (3×400 mL). The resulting mixture was concentrated under vacuum. The residue was purified by silica gel column chromatography, eluted with CH2Cl2/MeOH (100:1) to afford 344-1 (6 g, 51.27%) as an orange solid.

### 2. Synthesis of 344-2

Into a 250 mL 3-necked round-bottom flask were added 344-1 (3 g, 13,500 mmol, 1 equiv), bromocyclobutane (5.47 g, 40.500 mmol, 3 equiv), CuI (0.26 g, 1.350 mmol, 0.1 equiv), K2CO3 (3.73 g, 27.000 mmol, 2 equiv) and DMF (50 mL) at room temperature. The resulting mixture was stirred for overnight at 120° C. under nitrogen atmosphere. The resulting mixture was diluted with water (300 mL). The aqueous layer was extracted with EtOAc (3×150 mL). The resulting mixture was concentrated under vacuum. The residue was purified by Prep-TLC (CH2Cl2/MeOH 30:1) to afford 344-2 (300 mg, 7.64%) as a brown oil.

### 3. Synthesis of 344-3

Into an 8 mL sealed tube were added 344-2 (130 mg, 0.470 mmol, 1 equiv), EtOH (2 mL), H2O (0.5 mL), NH4Cl (125.83 mg, 2.350 mmol, 5 equiv) and Fe (78.82 mg, 1.410 mmol, 3 equiv) at room temperature. The resulting mixture was stirred for 6 h at 80° C. under nitrogen atmosphere. The resulting mixture was diluted with water (5 mL). The aqueous layer was extracted with EtOAc (3×5 mL). The resulting mixture was concentrated under vacuum. The residue was purified by Prep-TLC (FE/EA 2:1) to afford 344-3 (80 mg, 65.58%) as a light yellow oil.

### 4. Synthesis of 344-4

Into an 8 mL sealed tube were added 344-3 (125 mg, 0.507 mmol, 1 equiv), DCE (1.25 mL), −2 (290.56 mg, 1,014 mmol, 2 equiv) and NaBH(OAc)3 (322.65 mg, 1.521 mmol, 3 equiv) at room temperature. The resulting mixture was stirred for overnight at room temperature. The reaction was quenched by the addition of sat. NH4Cl (aq.) (10 mL) at room temperature. The resulting mixture was extracted with EtOAc (3×15 mL). The combined organic layers were washed with water (2×10 mL), dried over anhydrous Na2SO4. After filtration, the filtrate was concentrated under reduced pressure. The residue was purified by Prep-TLC (CH2Cl2/MeOH 15:1) to afford 344-4 (130 mg, 47.11%) as a light yellow oil.

### 5. Synthesis of 344

Into a 50 ml, 3-necked round-bottom flask were added 344-4 (130 mg, 0.252 mmol, 1 equiv), DCM (2.5 mL) and Pyridine (119.42 mg, 1.512 mmol, 6 equiv) at 0° C. To the above mixture was added Triphosgene (33.60 mg, 0.113 mmol, 0.45 equiv) at 0° C. The resulting mixture was stirred for additional 10 min at 0° C. The reaction was quenched by the addition of sat. NaHCO3 (aq.) (5 mL) at 0° C. The resulting mixture was extracted with CH2Cl2 (3×10 mL). The resulting mixture was concentrated under reduced pressure. The resulting mixture was concentrated under vacuum. The residue was purified by Prep-TLC (CH2Cl2/MeOH 15:1) to afford crude product (70 mg) as a light yellow oil. The crude product (70 mg) was purified by Prep-HPLC with the following conditions (Column: XBridge Prep EVO C18 Column, 30*150 mm, 5 μm; Mobile Phase A: Water (10 mmol/L NH4HCO3), Mobile Phase B: ACN; Flow rate: 60 mL/min; Gradient: 60% B to 90% B in 9 mi; Wave Length: 220 am; RT (min): 8.66) to afford 344 (44.4 mg, 24.02%) as a light yellow solid.

LCMS: (ES, m/z): [M+H]+ 544. H-NMR: (300 MHz, CD3OD, ppm): δ 0.81-0.89 (m, 4H), δ 1.46-1.49 (m, 1H), δ 1.58-1.66 (m, 4H), δ 1.76-1.91 (m, 3H), δ 2.31-2.42 (m, 2H), δ 2.43-2.49 (m, 2H), δ 2.67-2.77 (m, 2H), δ 3.24-3.30 (d, 2H), δ 5.09-5.13 (m, 1H), δ 6.95-7.00 (m, 2H), δ 7.37 (s, 1H), δ 7.43-7.45 (m, 1H), δ 7.47-7.52 (m, 2H), δ 7.66 (s, 1H), δ 8.36 (s, 1H).

### Example 345. Synthesis of Compound 345

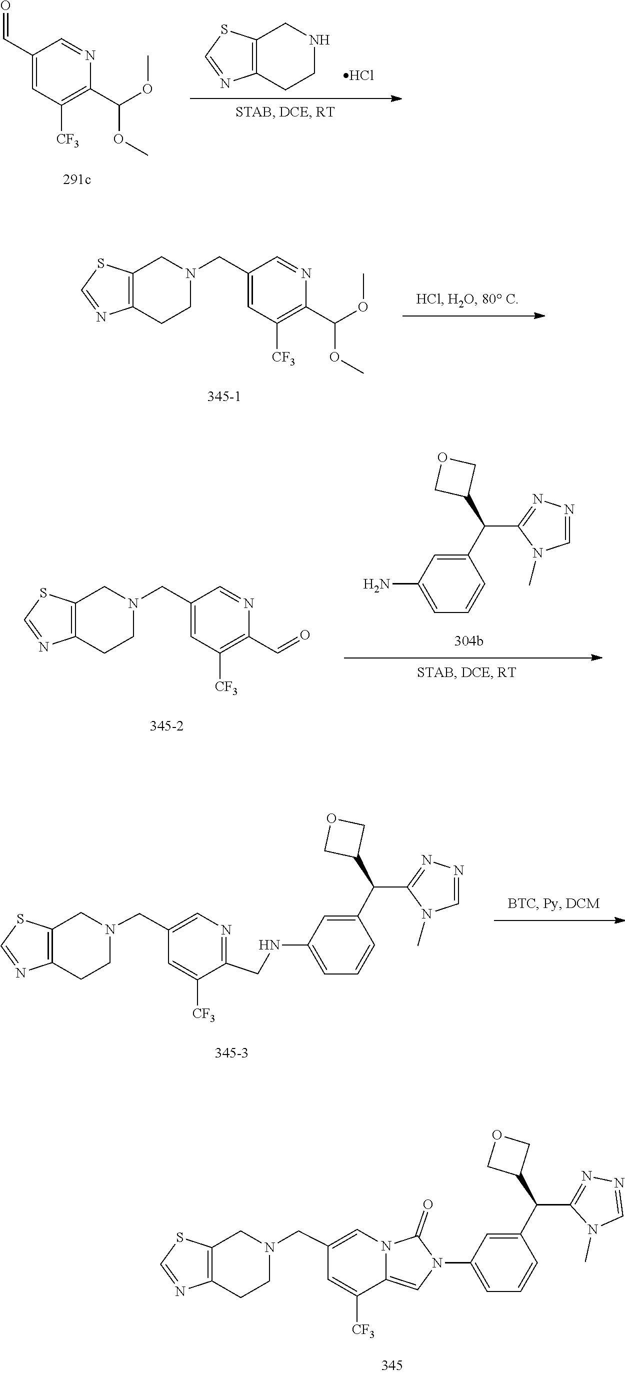

### 1. Synthesis of 345-1

Into a 20 mL sealed tube were added 291c (600 mg, 2.408 mmol, 1.00 equiv), DCE (8 mL) and 4H,5H,6H,7H-[1,3]thiazolo[5,4-c]pyridine hydrochloride (425.36 mg, 2.408 mmol, 1 equiv) and Et3N (486.41 mg, 4.816 mmol, 1 equiv) at room temperature. The resulting mixture was stirred for 4 h at room temperature. To the above mixture was added STAB (1020.62 mg, 4.816 mmol, 2 equiv) at room temperature. The resulting mixture was stirred for additional 2 h at room temperature. The reaction was quenched with sat. NaHCO₃ (aq.)(20 mL) at room temperature. The aqueous layer was extracted with CH2Cl2/MeOH (10:1) (2×15 mL). The resulting mixture was concentrated under vacuum. The residue was purified by Prep-TLC (CHCl2/MeOH=30:1) to afford 345-1 (400 mg, 44.49%) as a yellow oil.

### 2. Synthesis of 345-2

Into a 8 mL sealed tube were added 345-1 (400 mg, 1.071 mmol, 1 equiv) and HC (1 M, 4 mL) at room temperature. The resulting mixture was stirred for overnight at 80° C. The mixture was allowed to cool down to room temperature. The mixture was basified to pH 7 with saturated NaOH (aq.)(1 M). The aqueous layer was extracted with CH2Cl2/MeOH=10/1 (2×15 mL). The resulting mixture was concentrated under vacuum. The residue was purified by Prep-TLC (CH2Cl2/MeOH 20:1) to afford 345-2 (260 mg, 74.15%) as a yellow oil.

### 3. Synthesis of 345-3

To a stirred solution of 345-2 (450.20 mg, 1.375 mmol, 1.2 equiv) and 304b (280 mg, 1.146 mmol, 1.00 equiv) in DCE (15 mL) was added STAB (485.83 mg, 2.292 mmol, 2 equiv). The resulting mixture was stirred for overnight at room temperature. The reaction was quenched by the addition of sat. NaHCO₃ (aq.) (20 mL). The resulting mixture was extracted with CH2Cl2/MeOH (3×20 mL). The combined organic layers were concentrated under reduced pressure. The residue was purified by reverse flash chromatography with the following conditions: column, C18 silica gel; mobile phase, MeCN in water (10 mmol/L NH4HCO3) 5% to 55% gradient in 30 min; detector, UV 254 nm. This resulted in 345-3 (200 mg, 31.41%) as a white solid.

### 4. Synthesis of 345

To a stirred solution of 345-3 (140 mg, 0,252 mmol, 1 equiv) and Pyridine (119.59 mg 1.512 mmol, 6 equiv) in DCM (3 ml) was added Triphosgene (29.91 mg, 0.101 mmol, 0.4 equiv) at 0° C. The resulting mixture was stirred for 0.5 h at 0° C. The reaction was quenched by the addition of sat. NaHCO₃ (aq.) (10 mL). The resulting mixture was extracted with CH2Cl2/MeOH=10/1 (3×15 mL). The combined organic layers were concentrated under reduced pressure. The residue was purified by reverse flash chromatography with the following conditions: column, C18 silica gel; mobile phase, MeCN in water (10 mmol/L NH4HCO3), 15% to 65% gradient in 30 min; detector, UV 254 m. This resulted in 345 (99.8 mg, 68.10%) as a yellow solid.

LC-MS: (ES, m/z): [M+H]+ 582. H-NMR: (400 MHz, DMSO-d6, ppm): δ 2.82-2.88 (m, 4H), δ 3.38 (s, 3H), δ 3.53 (s, 2H), δ 3.75 (s, 2H), δ 3.90-3.99 (m, 1H), δ 4.27-4.30 (t, 1H), δ 4.49-4.50 (d, 2H), δ 4.73-4.82 (m, 2H), δ 7.07 (s, 1H), δ 7.18-7.20 (d 1H), δ 7.39 (s, 1H), δ 7.46-7.50 (t, 1H), δ 7.76-7.78 (m, 3H), δ 8.38 (s, 1H), δ 8.92 (s, 1H).

### Example 346. Synthesis of Compound 346

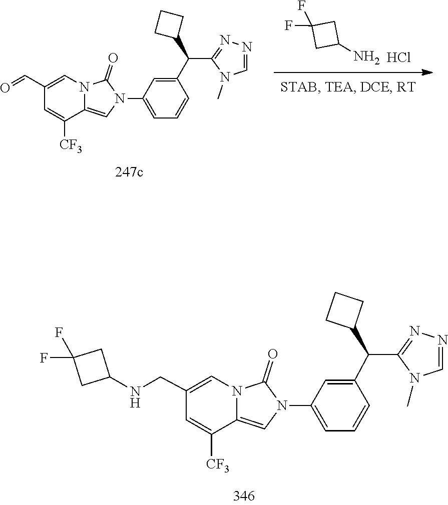 [SMILES: Cn1cnnc1[C@@H](c1cccc(-n2cc3c(C(F)(F)F)cc(C=O)cn3c2=O)c1)C1CCC1 ; NC1CC(F)(F)C1 ; Cl ; Cn1cnnc1[C@@H](c1cccc(-n2cc3c(C(F)(F)F)cc(CNC4CC(F)(F)C4)cn3c2=O)c1)C1CCC1]

### 1. Synthesis of 346

To a stirred solution of 247 C (300 mg, 0.659 mmol, 1.00 equiv) and 3,3-difluorocyclobutan-1-amine hydrochloride (189.13 mg, 1.318 mmol, 2 equiv) in DCE (4 mL) were added TEA (133.31 mg, 1.318 mmol, 2 equiv) at room temperature under air atmosphere. The mixture was stirred for 30 min at room temperature. To the above mixture was added STAB (279.21 mg, 1.318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×15 mL). The resulting mixture was concentrated under reduced pressure. The crude product (98 mg) was purified by Prep-HPLC with the following conditions (Column: X-Bridge Prep OED C18 Column, 30*150 mm, 5 μm; Mobile Phase A: Water (10 mmol/L NH4HCO3), Mobile Phase B: ACN; Flow rate: 60 mL/min; Gradient: 25% B to 55% B in 7 min, 55% B; Wave Length: 220 nm; RT1 (min): 8.13) to afford 346 (710 mg, 19.13%) as a yellow solid.

LC-MS: (ES, m/z): [M+H]+: 547. H-NMR: (400 MHz, DMSO-d6, ppm): δ 1.65-1.89 (m, 5H), δ 2.02-2.16 (m, 1H), δ 2.29-2.56 (m, 2H), δ 3.62-3.81 (m, 3H), δ 3.12-3.23 (m, 1H), δ 3.24-3.26 (m, 1H), δ 3.45-3.51 (m, 5H), δ 4.25-4.27 (d, 1H), δ 7.10 (s, 1H), δ 7.18-7.20 (d, 1H), δ 7.31 (s, 1H), δ 7.42-7.46 (m, 1H), δ 7.71-7.78 (m, 3H), δ 8.33 (s, 1H).

### Example 347. Synthesis of Compound 347

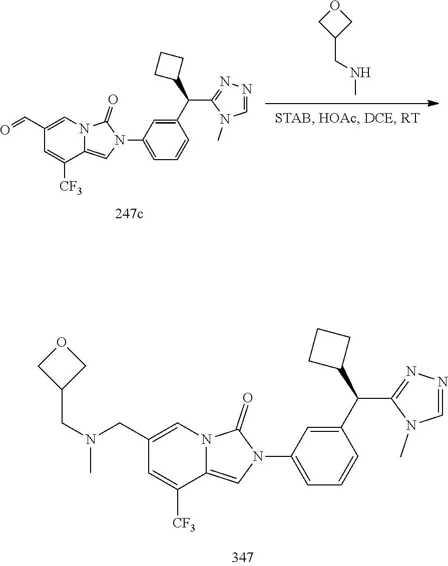

### 1. Synthesis of 347

To a stirred solution of 247c (300 mg, 0.659 mmol, 1 equiv) and methyl(oxetan-3-ylmethyl)amine (133.25 mg, 1.318 mmol, 2 equiv) in DCE (4 mL) were added STAB (279.21 mg, 1.318 mmol, 2 equiv) and HOAc (39.56 mg, 0.659 mmol, 1 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×20 mL). The resulting mixture was concentrated under reduced pressure. The crude product (52 mg) was purified by Prep-HPLC with the following conditions (Column: XSelect CSH Fluoro Phenyl, 30*150 mm, 5 μm; Mobile Phase A: Water (10 mmol/L NH4HCO3), Mobile Phase B: ACN; Flow rate: 60 mL/min; Gradient: 39% B to 53% B in 8 min; Wave Length: 254/220 nm; RT1 (min): 6.42) to afford 347 (36.7 mg, 10.00%) as a yellow solid.

LC-MS-347 (ES, m/z): [M+H]+: 541

H-NMR-347 (400 MHz, DMSO-d6, ppm): δ 1.62-1.89 (m, 5H), δ 2.01-2.16 (m, 4H), δ 2.63-2.72 (m, 2H), δ 3.12-3.23 (m, 2H), δ 3.33-3.36 (m, 2H), δ 3.45 (s, 3H), δ 4.22-4.31 (m, 3H), δ 4.61-4.64 (m, 2H), δ 7.00 (s, 1H), δ 7.18-7.20 (d, 1H), δ 7.31 (s, 1H), δ 7.42-7.46 (m, 1H), δ 7.71-7.78 (m, 3H), δ 8.33 (s, 1H).

### Example 348. Synthesis of Compound 348

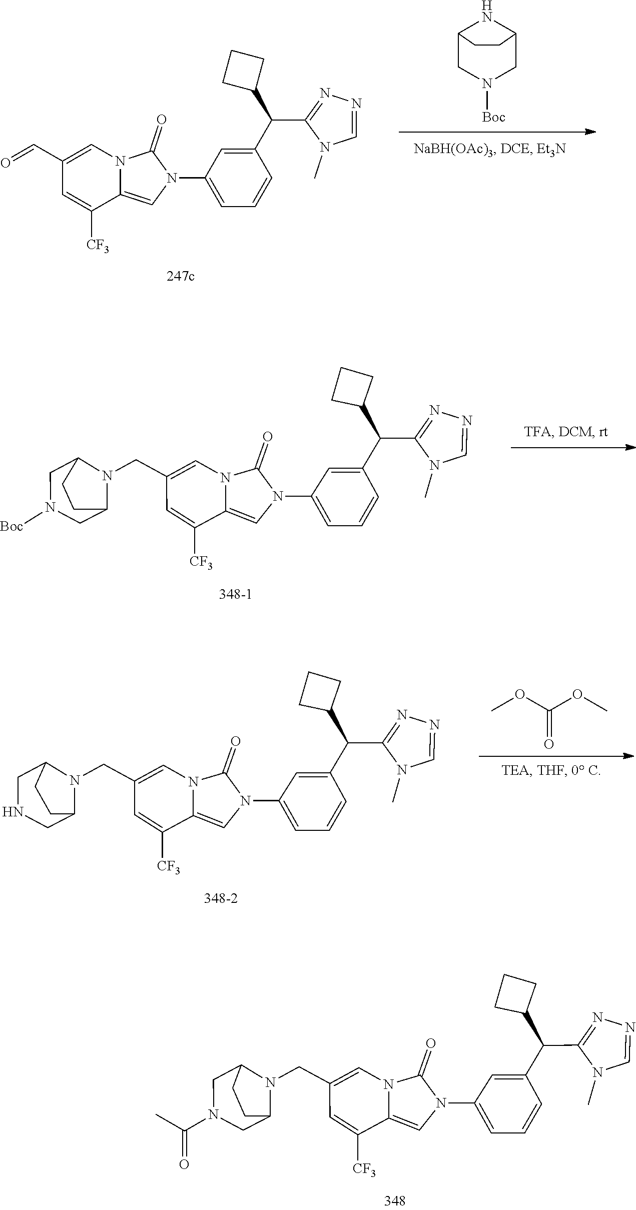

### 1. Synthesis of 348-1

To a stirred mixture of tert-butyl 3,8-diazabicyclo [3.2.1] octane-3-carboxylate (1165.32 mg, 5,490 mmol, 5 equiv) and Et3N (166.64 mg, 1,647 mmol, 1.5 equiv) in DCE (5 mL) were added 247 C (500 mg, 1.098 mol, 1 equiv) and NaBH(OAc)3 (698.03 mg, 3,294 mmol, 3 equiv) at room temperature. The resulting mixture was stirred for overnight at room temperature. The reaction was quenched with sat, NH4Cl (aq.) (20 ml) at room temperature. The aqueous layer was extracted with EtOAc (3×10 mL). The resulting mixture was concentrated under reduced pressure. The residue was purified by Prep-TLC (C H2Cl2/MeOH 10:1) to afford 348-1 (340 mg, 42.77%) as a yellow oil.

### 2. Synthesis of 348-2

A solution of 348-1 (320 mg, 0,442 mmol, 1 equiv, 90%) in TFA (1 mL) and DCM (3 mL) was stirred for overnight at room temperature. The mixture was neutralized to pH 7 with saturated NaHCO3 (aq.). The aqueous layer was extracted with CH2Cl2 (3×10 mL). The resulting mixture was concentrated under reduced pressure. The residue was purified by Prep-TLC (CH2Cl2/MeOH 10:1) to afford 348-2 (220 mg, 85.74%) as a yellow solid.

### 3. Synthesis of 348

To a stirred mixture of 348-2 (210 mg, 0.362 mmol, 1 equiv, 95%) and dimethyl carbonate (32.58 mg, 0362 mmol), 1 equiv) in THF (2 mL) was added TEA (73.20 mg, 0.724 mmol, 2 equiv) at 0° C. The resulting mixture was stirred for 2o at v° C. The mixture was neutralized to pH 7 with saturated NaHCO₃ (aq.). The aqueous layer was extracted with CH2Cl2: MeOH (10:1) (3×10 mL). The resulting mixture was concentrated under reduced pressure. The residue was purified by reverse flash chromatography with the following conditions: Column: C18 Column 40 g; Mobile Phase A: Water (0.1% N14HCO3), Mobile Phase B: ACN; Flow rate: 60 mL/min; Gradient: 31% B to 61% B in 8 min to afford 348 (116.9 mg, 52.98%) as a yellow solid.

LC-MS-348 (ES, m/z): [M+H]+ 594

H-NMR: (400 MHz, DMSO, ppm): δ 1.25-1.31 (m, 1H), δ 1.31-1.49 (m, 1H), δ 1.65-1.75 (m, 1H), δ 1.75-1.88 (m, 4H), δ 1.88-1.95 (d, 2H), δ 1.95-2.01 (d, 3H), δ 2.05-2.15 (m, 1H), δ 2.66-2.69 (d, 1H), δ 3.19-3.26 (m, 4H), δ 3.26-3.32 (m, 2H), δ 3.40-3.55 (m, 4H), δ 3.84-4.15 (d, 1H), δ 4.25-4.35 (d, 1H), δ 7.10 (s, 1H), δ 7.19-7.21 (d, 1H), δ 7.32 (s, 1H), δ 7.42-7.47 (m, 1H), δ 7.68-7.71 (m, 1H), δ 7.73-7.76 (d, 2H), δ 8.33 (s, 1H),

### Example 349. Synthesis of Compound 349

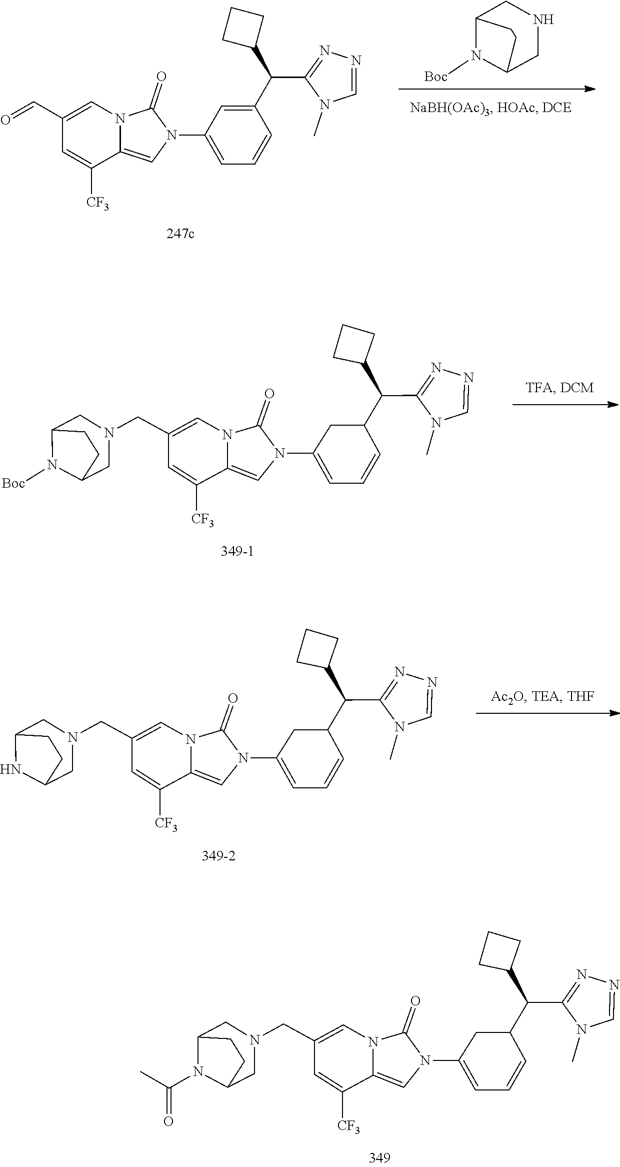 [SMILES: Cn1cnnc1[C@@H](c1cccc(-n2cc3c(C(F)(F)F)cc(C=O)cn3c2=O)c1)C1CCC1 ; CC(C)(C)OC(=O)N1C2CCC1CNC2 ; Cn1cnnc1[C@@H](C1C=CC=C(n2cc3c(C(F)(F)F)cc(CN4CC5CCC(C4)N5C(=O)OC(C)(C)C)cn3c2=O)C1)C1CCC1 ; Cn1cnnc1[C@@H](C1C=CC=C(n2cc3c(C(F)(F)F)cc(CN4CC5CCC(C4)N5)cn3c2=O)C1)C1CCC1 ; CC(=O)N1C2CCC1CN(Cc1cc(C(F)(F)F)c3cn(C4=CC=CC([C@H](c5nncn5C)C5CCC5)C4)c(=O)n3c1)C2]

### 1. Synthesis of 349-1

To a stirred solution of 247c (500 mg, 1.09 mmol, 1.0 equiv) and tert-butyl 3,8-diazabicyclo [3.2.1] octane-8-carboxylate (233 mg, 1.09 mmol, 1.1 equiv) in DCE (5 mL) were added HOAc (65 mg, 1.09 mmol, 1.0 equiv) and NaBH(OAc)3 (465 mg, 2.19 mmol, 2.0 equiv) at room temperature. The resulting mixture was stirred overnight at room temperature. The reaction was quenched with water at room temperature. The resulting mixture was extracted with CH2Cl2 (3×20 mL). The combined organic layers were concentrated under reduced pressure. The residue was purified by Prep-TLC (CH2Cl2/MeOH=1:1) to afford 349-1 (380 mg, 48%) as a yellow solid.

### 2. Synthesis of 349-2

A solution of 349-1 (380 mg, 0.58 mmol, 1.0 equiv) and TFA (1 mL) in DCM (3 mL) was stirred overnight at room temperature. The resulting mixture was concentrated under reduced pressure. The mixture was neutralized to pH 7 with NH3 in MeOH. The residue was purified by Prep-TLC (CH2Cl2/MeOH=10:1) to afford 349-2 (240 mg, 73%) as a yellow solid.

### 3. Synthesis of 349-0

To a stirred solution of 349-2 (240 mg 0.43 mmol, 1.0 equiv) and TEA (88 mg, 0.87 mmol, 2.0 equiv) in THF (2.4 mL) was added Ac2O (44 mg, 0.43 mmol, 1.0 equiv) dropwise at 0° C. The resulting mixture was stirred for 1 h at room temperature. The reaction was quenched with water at room temperature. The resulting mixture was extracted with EtOAc (3×20 mL). The combined organic layers were concentrated under reduced pressure. The residue was purified by Prep-TLC (CH2Cl2/MeOH=10:1) to afford 349 (189 mg, 72%) as a yellow solid.

LC-MS-349 (ES, m/z): [M/2+H]+297

H-NMR: (400 MHz, dmso-d6, δ ppm): 1.58-1.96 (m, 9H), 1.96 (s, 3H), 2.07-2.09 (m, 1H), 2.13-2.22 (m, 2H), 2.65-2.72 (m, 2H), 3.20-3.25 (m, 1H), 3.30-3.33 (m, 2H), 3.43 (s, 3H), 4.16 (s, 1H), 4.25-4.27 (d, 1H), 4.41-4.43 (d, 1H), 7.06 (s, 1H), 7.18-7.20 (d, 1H), 7.32 (s, 1H), 7.42-7.46 (t, 1H), 7.67-7.73 (m, 3H), 8.33 (s, 1H).

### Example 350. Synthesis of Compound 350

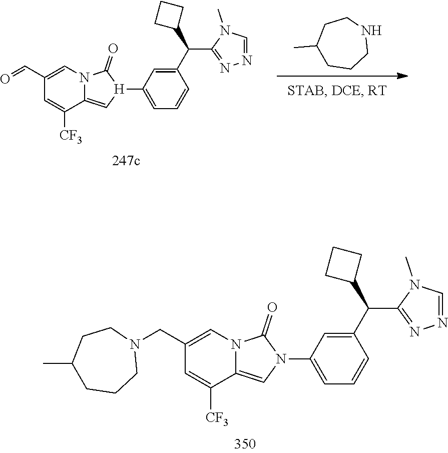

### 1. Synthesis of 350

To a stirred mixture of 247c (500 mg, 1,098 mmol, 1 equiv) and 4-methylazepane (248.56 mg, 2.196 mmol, 2 equiv) in DCE (5 mL) was added NaBH(OAc)3 (698.03 mg, 3,294 mmol, 3 equiv) at room temperature. The resulting mixture was stirred overnight at room temperature. The reaction was quenched by the addition of water (50 mL) at room temperature. The aqueous layer was extracted with DCM (2×20 mL). The resulting mixture was concentrated under reduced pressure. The residue was purified by Prep-TLC (CH2Cl2/MeOH=15:1) to afford 350 (64.4 mg, 10.49%) as a yellow solid.

LCMS: (ES, m/z): [M+H]+ 553. H-NMR: (400 MHz, CD3OD-d4, ppm, S): 0.97-1.01 (s, 3H), 1.3 (m, 3H), 2.84-2.89 (m, 1H), 3.21-3.29 (s, 3H), 3.55 (s, 2H), 3.80 (s, 2H), 7.07 (s, 1H), 7.25-7.27 (d, 1H), 7.39 (s, 1H), 7.48-7.52 (m, 1H), 7.64-7.66 (m, 1H), 7.77 (s, 1H), 7.82 (s, 1H), 8.30 (m, 3H), 8.92 (s, 1H).

### Example 351. Synthesis of Compound 351_P1 and 351_P2

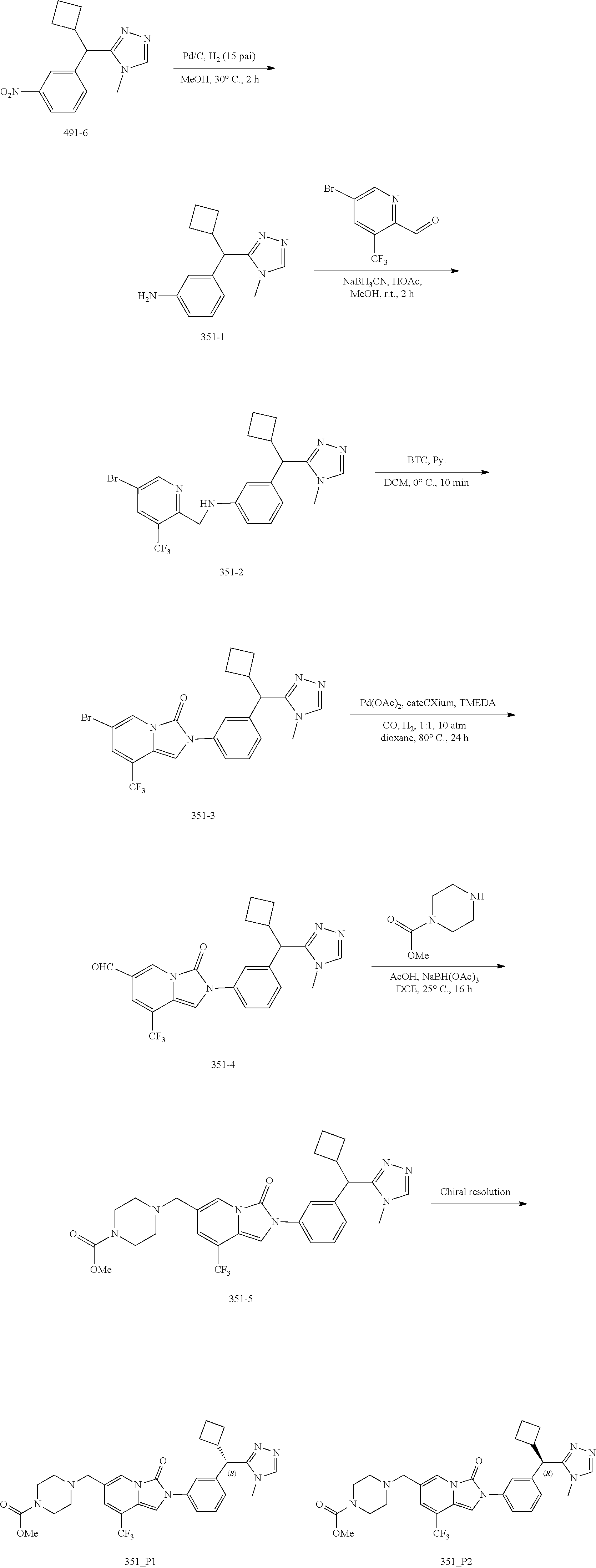

### 1. Synthesis of 351-1

To a solution of 491-6 (9 g, 33 mmol, 1 equiv) in MeOH (150 mL) was added Pd/C (3 g, 10% purity) under nitrogen atmosphere. The suspension was degassed under vacuum and purged with H2 several times. The mixture was stirred under H2 (15 psi) at 25° C. for 12 hours. The reaction mixture was filtered and the filter was concentrated to give crude product. The crude product was triturated with PE (100 mL) at 25° C. for 0.5 h. The mixture was filtered and the filter cake was concentrate in vacuum to afford 351-1 (7 g, crude) as a yellow solid,

H-NMR-351-1: (400 MHz, DMSO-d6) δ 8.29 (s, 1H), 6.92 (t, J=7.69 Hz, 1H), 6.29-6.44 (m, 3H), 5.01 (s, 2H), 3.92 (d, J=10.4 Hz, 1H), 3.35 (s, 3H), 3.10 (t, J=7.6 Hz, 1H), 1.99-2.16 (m, 1H), 1.71-1.85 (m, 4H), 1.57-1.71 (m, 1H)

### 2. Synthesis of 351-2

To a solution of 351-1 (12 g, 49.52 mmol, 1 equiv) in DCE (120 mL) was added HOAc (2.83 ml, 49.5 mmol, 1 equiv) and 5-bromo-3-(trifluoromethyl)picolinaldehyde (15.1 g, 59.4 mmol, 1.2 equiv) at 25° C., After addition, the mixture was stirred at 25° C. for 2 hrs, and then NaBH(OAc)3 (20.9 g, 99.0 mmol, 2 equiv) was added. The resulting mixture was stirred at 25° C. for 2 hrs. The reaction was poured into Sat.NaHCO₃ (100 mL) and extracted with CH2Cl2 (2×100 ml). The organic phase was washed with brine (80 mL), dried over anhydrous Na2SO4, filtered and concentrated in vacuum to give crude product. The crude product was triturated with PE (100 mL) at 25° C. for 10 min, filtered and the filter cake was concentrated in vacuum to afford 351-2 (17 g, 66%)

H-NMR-351-2: (400 MHz, DMSO-d6) δ 8.91-9.03 (m, 1H), 8.47 (d, J=2.0 Hz, 1H), 8.29 (s, 1H), 6.93-7.04 (m, 1H), 6.35-6.52 (m, 3H), 6.23 (s, 1H), 4.40-4.51 (m, 2H), 3.89-4.00 (m, 1H), 3.04-3.19 (m, 1H), 2.02-2.15 (m, 1H), 1.59-1.86 (m, 5H)

### 3. Synthesis of 351-3

To a solution of 351-2 (6 g, 12.49 mmol, 1 equiv) and pyridine (6.05 ML, 74.9 mmol, 6 equiv) in DCM (60 mL) was added TRIPHOSGENE (1.67 g, 5.62 mmol, 0.45 equiv) in DCM (6 mL) at 0° C. The mixture was stirred at 0° C. for 10 min. The reaction was poured into sat NaHCO3 (600 mL) and the resulting mixture was extracted with EtOAc (2×300 mL). The organic phase was washed with brine (80 mL), dried over anhydrous Na2SO4, concentrated in vacuum to give crude product. The crude product was triturated with PE (50 mL) at 25° C. for 10 min, filtered and the filter cake was concentrated in vacuum to afford 351-3 (6 g, 95%).

H-NMR-351-3: (400 MHz, DMSO-d6) δ 8.54-8.65 (m, 1H), 8.33 (s, 1H), 8.02 (s, 1H), 7.73-7.76 (m, 1H), 7.65-7.71 (m, 1H), 7.41-7.49 (m, 2H), 7.19-7.25 (m, 1H), 7.16 (s, 1H), 4.19-4.34 (m, 1H), 3.43 (s, 3H), 3.14-3.27 (m, 1H), 2.03-2.16 (m, 1H), 1.63-1.89 (m, 5H)

### 4. Synthesis of 351-4

To a solution of 351-3 (340 mg, 671 umol, 1 equiv) in dioxane (20 mL) was added N,N,N′,N′-tetramethylethane-1,2-diamine (202 uL, 1.34 mmol, 2 equiv) and diacetoxypalladium (30.1 mg, 134 umol, 0.2 equiv), bis(1-adamantyl)-butyl-phosphane (48.5 mg, 134 umol, 0.2 equiv). The mixture was stirred at 80° C. for 12 hr under CO/H2 (1:1) (2.5 Mpa). The reaction mixture was concentrated in vacuum to dryness. The reaction was poured into water (50 mL) and the resulting mixture was extracted with EtOAc (2×50 mL). The organic phase was washed with sat NaHCO3 (20 mL), brine (20 mL), dried over anhydrous Na2SO4, concentrated in vacuum to afford 351-4 (450 mg, crude) as yellow solid, which was used into the next step without further purification.

### 5. Synthesis of 351-5

To a solution of 351-4 (300 mg, 658 umol, 1.0 equiv) and methyl piperazine-1-carboxylate (284 mg, 1.98 mmol, 3.0 equiv) in DCE (10 mL) was added AcOH (39 mg, 658 umol, 1.0 equiv) at. After addition, the mixture was stirred at 25 CC for 2 hrs, and then NaBH(OAc)3 (698 mg, 3.29 mmol, 5.0 equiv) was added. The resulting mixture was stirred at 25° C. for 14 hrs. The reaction was poured into water (10 mL) and the resulting mixture was extracted with CH12Cl2 (2×10 mL). The organic phase was washed with brine (10 mL), dried over anhydrous Na2SO4. After filtration, the filtrate was concentrated in vacuum to give a residue. The residue was purified by prep-HPLC (column: Waters Xbridge BEH C18 100*30 mm*10 um; Mobile Phase A: Water (NO-41-CO3), Mobile Phase B: ACN; Plow rate: 50 mL/in; Gradient: 25% B to 55% B in 8 min; Wave Length: 220 nm; RT1 (min): 5.1) to afford 351-5 (74 mg, 17% yield) as a yellow solid.

### 6. Synthesis of 351_P1& P2

351-5 (74 mg) was purified by Chiral separation with the following conditions (column: DAICEL CHIRALCEL OD (250 mm*30 mm, 10 um); Mobile Phase: [Neu-PA]; B %: 50%-50%, 10 min, Flow rate: 70 mL/min; Wave Length: 220/254 nm; RT1 (min): 1.77 RT2 (min): 2.56) to afford 351_P1 (13 mg, 33% yield) as a yellow solid.

MS-351-P1: (ES, m/z): [M+H]+ 584.3.

H-NMR-351-P1: (400 MHz, DMSO-d6) δ 8.32 (s, 1H), 7.72 (s, 1H), 7.65-7.74 (m, 2H), 7.42-7.45 (m, 1H), 7.28-7.32 (m, 1H), 7.18-7.20 (m, 1H), 7.01 (s, 1H), 4.25 (d, J=5.2 Hz, 1H), 3.58 (s, 3H), 3.42 (s, 3H) 3.30-3.30-3.31 (m, 4H), 3.27-3.29 (m, 2H), 3.24-3.25 (m, 1H), 2.35-2.39 (m, 4H), 2.09-2.13 (m, 1H), 1.76-1.79 (m, 4H), 1.69-1.70 (m, 1H).

351-P2 (21 mg, 56% yield) as a yellow solid.

MS-351-P2: (ES, m/z): [M+H]+ 584.3.

H-NMR-351-P2: (400 MHz, DMSO-d6) δ 8.32 (s, 1H), 7.72 (s, 1H), 7.65-7.74 (m, 2H), 7.42-7.45 (m, 1H), 7.28-7.32 (m, 1H), 7.18-7.20 (m, 1H), 7.01 (s, 1H), 4.25 (d, J=5.2 Hz, 1H), 3.58 (s, 3H), 3.42 (s, 3H) 3.30-3.30-3.31 (m, 4H), 3.27-3.29 (m, 2H), 3.24-3.25 (m, 1H), 2.35-2.39 (m, 4H), 2.09-2.13 (m, 1H), 1.76-1.79 (m, 4H), 1.69-1.70 (m, 1H).

### Example 352. Synthesis of Compound 352_P1 and 352_P2

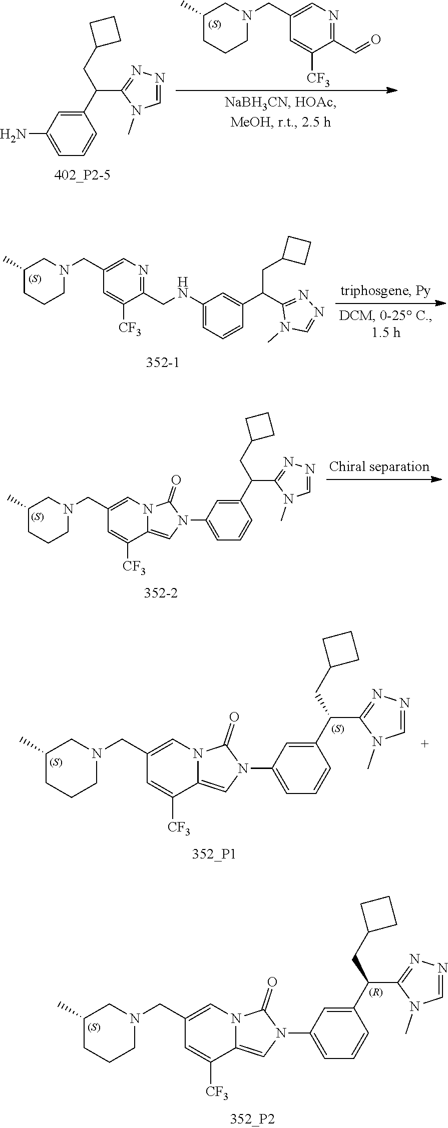

### 1. Synthesis of 352-1

To a solution of 402_P2-6 (1 g, 3.9 mmol, 1 equiv) and 1-2 (1.01 g, 3.51 mmol, 0.9 equiv) in MeOH (20 mL) was added AcOH (669 ul, 11.7 mmol, 3 equiv), the mixture was stirred at 25° C. for 1 hr under nitrogen atmosphere, then NaBH3CN (490 mg, 7.8 mmol, 2 equiv) was added to the mixture, the result mixture was stirred at 25° C. for 1.5 hr under nitrogen atmosphere. The reaction mixture was diluted with Sat. NaHCO₃ (20 mL), extracted with EtOAc (20 mL×3). The combined organic layers were washed with brine (20 mL×2), dried over anhydrous Na2SO4, filtered and concentrated in vacuum. The residue was purified by silica gel column chromatography eluted with DCM/MeOH (10:1) to afford 352-1 (1.3 g, 57%) as a yellow oil H-NMR: (400 MHz, METHANOL-d4) 8.69 (s, 1H), 831 (s, 11), 8.13 (s, 1H), 7.19-6.97 (m, 1H), 6.71-6.55 (in, 11), 6.53-6.45 (m, 2H), 4.55 (s, 2H), 4.02-3.86 (m, 1H), 3.66 (s, 2H), 3.41 (s, 3H), 2.89-2.79 (in, 2H), 2.38-2.12 (m, 411) 2.10-2.00 (m, 2H), 1.93-1.86 (m, 1H), 1.80-1.62 (m, 8H), 0.99-0.92 (m, 1H), 0.89 (d, J=6.4 Hz, 3H)

### 2. Synthesis of 352-2

To a solution of 352-1 (1.2 g, 2.28 mmol, 1 equiv) in DCM (20 mL) was added pyridine (1.1 mL, 13.6 mmol, 6 equiv) and bis(trichloromethyl) carbonate (676 mg, 2.28 mmol, 1 equiv) at 0° C., then the mixture was stirred at 25° C. for 1.5 hr under nitrogen atmosphere. The reaction mixture was dilated with Sat. NaHCO3 (40 mL), extracted with DCM (25 mL×3). The combined organic layers were dried over anhydrous Na2SO4, filtered and concentrated in vacuum The residue was purified by silica gel column chromatography eluted with DCM/MeOH (10:1) to give crude product. The crude product (450 mg) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 25% B to 60% B in 8 min; Wave Length: 220 nm; RT1 (min): 6.0) to afford 352-2 (190 mg, 14.6%) as a yellow solid, H-NMR-352-2: (400 MHz, METHANOL-d4) δ 8.37 (s, 1H), 7.77 (s, 1H), 7.70-7.62 (m, 2K), 7.51 (t, J=8.0 Hz, 1H), 7.29 (d, J=8.0 Hz, 1H), 7.18 (s, 1H) 7.13 (s, 1H), 4.60 (s, 1H), 4.29-4.15 (in, 11), 3.58 (s, 2H), 3.55

### 3. Synthesis of 352_P1&P2

The 332-8 (250 mg) was purified by Chiral separation with the following conditions (column: DAICEL CHIRALCEL OD (250 mm*30 mm, 10 um); Mobile Phase: [IPA]; B %: 50%-50%, 8 min, Flow rate: 70 mL/min; Wave Length: 220/254 nm; RT1 (min): 3.63; RT2 (min): 4.77) to afford 352_P1 (50.4 mg, 36.4%) and 352 P2 (20.7 mg, 14.7%)

LCMS-352_P1: (ES, m/z): [M+H]+ 553.2. H-NMR-352_P1: (400 MHz, METHANOL-d4) δ 8.37 (s, 1H), 7.68 (d, J=2.0 Hz, 2H), 7.67-7.63 (m, 1H), 7.51 (t, J=8.0 Hz, 1H), 7.28 (d, J=8.0 Hz, 1H), 7.15 (s, 1H), 7.13 (s, 1H), 4.26-4.19 (m, 1H), 3.54 (s, 3H), 3.35 (s, 2H), 2.94-2.83 (m, 2H), 2.48-2.39 (m, 1H), 2.33-2.24 (m, 2H), 2.12-2.06 (m, 1H), 2.03-1.90 (m, 2H), 1.88-1.57 (m, 9H), 0.95-0.89 (m, 3H)

LCMS-352_P2: (ES, m/z): [M+H]+ 553.2. H-NMR-352_P2: (400 MHz, METHANOL-d4) δ 8.37 (s, 1H), 7.68 (d, J=1.2 Hz, 2H), 7.67-7.63 (m, 1H), 7.51 (t, J=8.0 Hz, 1H), 7.28 (d, J=8.0 Hz, 1H), 7.15 (s, 1H), 7.13 (s, 1H), 4.26-4.18 (m, 1H), 3.54 (s, 3H), 3.35 (s, 2H), 2.98-2.80 (m, 2H), 2.46-2.39 (m, 1H), 2.35-2.22 (m, 2H), 2.13-2.04 (m, 1H), 2.02-1.90 (m, 2H), 1.88-1.58 (m, 9H), 1.00-0.88 (m, 4H)

### Example 353. Synthesis of Compound 353

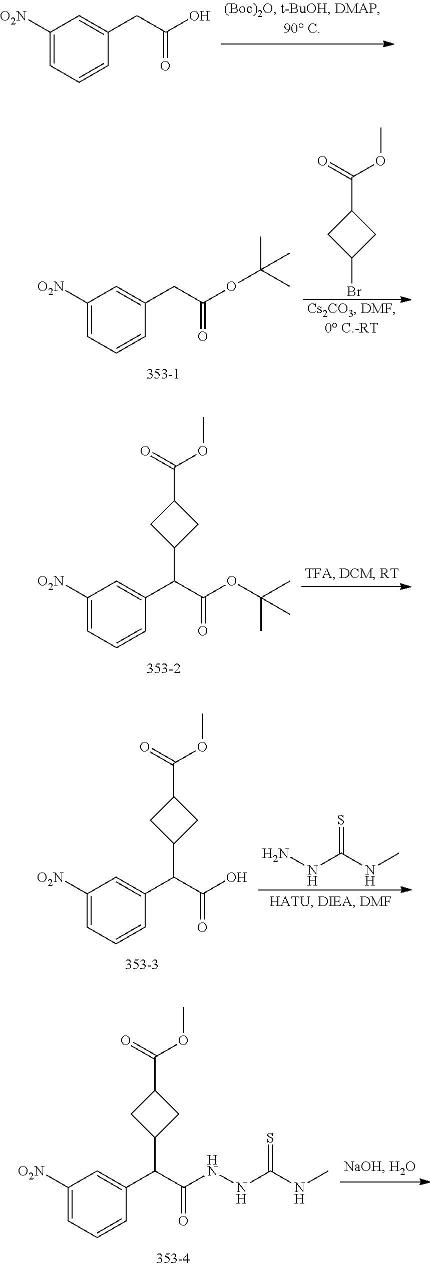

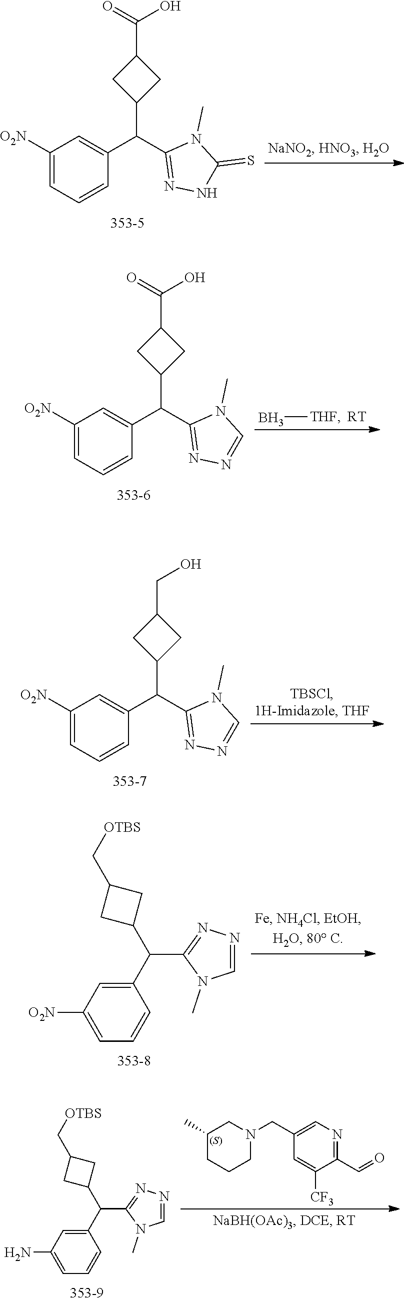

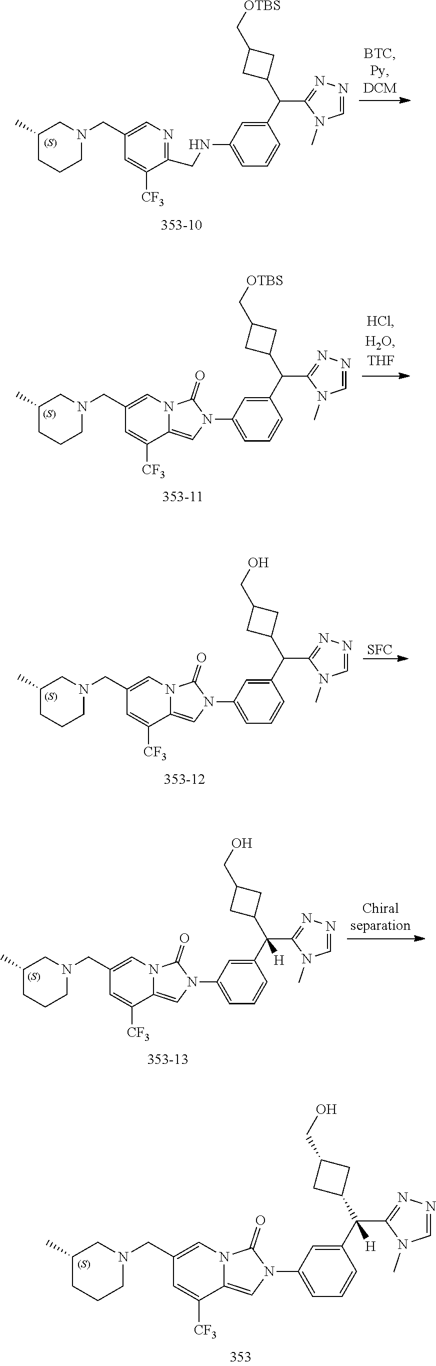

### 1. Synthesis of 353-1

To a stirred solution of 2-(3-nitrophenyl)acetic acid (50 g, 276.019 mmol, 1 equiv) and DMAP (337 g, 27,602 mmol, 0.1 equiv) in t-BuOH (100 mL) was added (Boc)20 (120.48 g, 552.038 mmol, 2 equiv) at room temperature under nitrogen atmosphere. The resulting mixture was stirred for 6 h at 90° C. under nitrogen atmosphere. The reaction was quenched with water (500 mL) at room temperature. The aqueous layer was extracted with EtOAc (2×200 mL). The resulting mixture was concentrated under vacuum. The residue was purified by silica gel column chromatography, eluted with PE/EA (50:1) to afford 353-1 (60 g, 82.46%) as a light yellow oil.

### 2. Synthesis of 353-2

To a stirred solution of 353-1 (8 g, 33719 mmol, 1 equiv) in DMF (100 mL) was added Cs2CO3 (54.93 g, 168,595 mmol, 5 equiv) at 0° C. under nitrogen atmosphere. The resulting mixture was stirred for 2 h at 0° C. To the above mixture was added methyl 3-bromocyclobutane-1-carboxylate (19.53 g, 101.157 mmol, 3.00 equiv) at 0° C. The resulting mixture was stirred for additional overnight at room temperature. The reaction was quenched with saturated NH4Cl (aq.) (500 mL) at room temperature. The aqueous layer was extracted with EtOAc (2×200 mL). The resulting mixture was concentrated under vacuum. The residue was purified by silica gel column chromatography, eluted with PB/EA (10:1) to afford 353-2 (8 g, 61.12%) as a light yellow oil.

### 3. Synthesis of 353-3

To a stirred solution of 353-2 (8 g, 22.898 mmol, 1 equiv) in DCM (60 mL) was added TFA (20 mL) at room temperature. The resulting mixture was stirred overnight at room temperature. The resulting mixture was concentrated under vacuum. The crude product was used in the next step directly without further purification. This resulted in 353-3 (8 g, 95.313%) as a light brown oil.

### 4. Synthesis of 353-4

To a stirred solution of 353-3 (8 g, 27.278 mmol, 1 equiv) and HATU (15.56 g, 40.917 mmol, 1.5 equiv) in DMF (100 ml) were added DIEA (10.58 g, 81.834 mmol, 3 equiv) and 1-amino-3-methylthiourea (3.73 g, 35.461 mmol, 1.3 equiv) at room temperature. The resulting mixture was stirred for 3 h at room temperature. The resulting mixture was diluted with water (300 mL). The aqueous layer was extracted with EtOAc (2×200 mL). The resulting mixture was concentrated under vacuum. The residue was purified by silica gel column chromatography, eluted with PE/EA (1:1) to afford 353-4 (4.5 g, 36.86%) as a brown yellow oil.

### 5. Synthesis of 353-5

To a stirred solution of NaOH (3.79 g, 94.632 mmol, 8 equiv) in H2O (100 mL) was added 353-4 (4.5 g, 11.829 mmol, 1 equiv) at room temperature. The resulting mixture was stirred for 6 h at room temperature. The mixture/residue was neutralized to pH 7 with HCl (aq.). The resulting mixture was concentrated under vacuum. The crude product was used in the next step directly without further purification. This resulted in 353-5 (4 g, 77.65%) as a light brown oil.

### 6. Synthesis of 353-6

To a stirred solution of 353-5 (4 g, 11.482 mmol, 1 equiv) and NaNO2 (7.92 g, 114.820 mmol, 10 equiv) in 1120 (100 mL) was added 1N03 (114 mL, 114.820 mmol, 10 equiv, 1M) at room temperature. The resulting mixture was stirred for 4 h at room temperature. The mixture was neutralized to pH 7 with saturated NaHCO₃ (aq.). The resulting mixture was concentrated under vacuum. The residue was dissolved in DCM/MeOH=10:1 (300 mL). The resulting mixture was filtered, the filter cake was washed with DCM (2×50 mL). The filtrate was concentrated under reduced pressure. The crude product was used in the next step directly without further purification. This resulted in 353-6 (2 g, 49.56%) as a yellow solid.

### 7. Synthesis of 353-7

To a stirred solution of 353-6 (2 g, 6323 mmol, 1 equiv) in THF (30 mL) was added BH3-THF (31.61 mL, 31.615 mmol, 5 equiv) dropwise at 0° C. under nitrogen atmosphere. The resulting mixture was stirred for overnight at room temperature. The reaction was quenched with saturated NH4Cl (aq.) (300 mL) at room temperature. The aqueous layer was extracted with EtOAc (3×100 mL). The combined organic layers were dried over anhydrous Na2SO4. After filtration, the filtrate was concentrated under reduced pressure. The crude product was used in the next step directly without further purification. This resulted in 353-7 (2 g, 83.70%) as a light yellow solid.

### 8. Synthesis of 353-8

To a stirred solution of 353-7 (2 g, 6.615 mmol, 1 equiv) and TBSCl (2.99 g, 19.845 mmol, 3 equiv) in THF (50 mL) was added 1H-imidazole (2.25 g, 33.075 mmol, 5 equiv) at room temperature. The resulting mixture was stirred for 6 h at room temperature. The reaction was quenched with water (200 mL) at room temperature. The aqueous layer was extracted with EtOAc (2×100 mL). The resulting mixture was concentrated under vacuum. The residue was purified by silica gel column chromatography, eluted with PE/EA (5:1) to afford 353-8 (1.8 g, 60.74%) as an off-white solid.

### 9. Synthesis of 353-9

To a stirred solution of 353-8 (1.8 g 4.321 mmol, 1 equiv) and Fe (1.21 g, 21.605 mmol, 5 equiv) in EtOH (50 mL)/1H2O (10 mL) was added NH4Cl (2.31 g, 43.210 mmol, 10 equiv) at room temperature. The resulting mixture was stirred for 4 h at 80° C. The resulting mixture was concentrated under vacuum. The residue was dissolved in DCM (100 mL). The resulting mixture was filtered, the filter cake was washed with DCM (2×10 mL). The filtrate was concentrated under reduced pressure. The residue was purified by Prep-TLC (DCM/MeOH 10:1) to afford 353-9 (1.2 g, 67.53%) as an off-white solid.

### 10. Synthesis of 353-10

To a stirred solution of 353-9 (1.2 g, 3.104 mmol, 1 equiv) and I-2 (1.07 g, 3.725 mmol, 1.2 equiv) in DCE (30 mL) was added NaBH(OAc)3 (1.97 g, 9.312 mmol, 3 equiv) at room temperature. The resulting mixture was stirred for 6 h at room temperature. The reaction was quenched with saturated NaHCO₃ (aq.) (100 mL) at room temperature. The aqueous layer was extracted with DCM (2×100 mL). The resulting mixture was concentrated under vacuum. The residue was purified by silica gel column chromatography, eluted with DCM/MeOH (20:1) to afford 353-10 (1.7 g, 80.87%) as an off-white solid.

### 11. Synthesis of 353-11

To a stirred solution of 353-10 (1.7 g, 2,588 mmol, 1 equiv) and Pyridine (2.05 g, 25.880 mmol, 10 equiv) in DCM (40 mL) was added Triphosgene (0.31 g, 1.035 mmol, 0.4 equiv) at room temperature. The resulting mixture was stirred for 10 min at room temperature. The reaction was quenched with saturated NaHCO3 (aq.) (100 mL) at room temperature. The aqueous layer was extracted with DCM (2×50 mL). The resulting mixture was concentrated under vacuum. The crude product was used in the next step directly without further purification. This resulted in 353-11 (1.5 g, 78.94%) as a yellow solid.

### 12. Synthesis of 353-12

To a stirred solution of 353-11 (1.5 g, 2.196 mmol, 1 equiv) in THF (10 mL) was added HCl in H2O (20 mL, 1M) at room temperature. The resulting mixture was stirred for 1 b at room temperature. The mixture was neutralized to pH 7 with saturated NaHCO3 (aq.). The aqueous layer was extracted with DCM (2×100 mL). The resulting mixture was concentrated under vacuum. The residue was purified by Prep-TLC (DCM/MeOH 10:1) to afford 353-12 (1.1 g, 85.43%) as a yellow solid.

### 13. Synthesis of 353-13

The 353-12 (1.1 g, 1.934 mmol, 1 equiv) was purified by Prep-SFC with the following conditions (Column: CHIRAL ART Cellulose-SB, 3*25 cm, 5 μm; Mobile Phase A: CO2, Mobile Phase B: IPA (0.5% 2M NH3-MeOH); Flow rate: 100 mL/min; Gradient: isocratic 40% B; Column Temperature (° C.): 35; Back Pressure (bar): 100; Wave Length: 220 nm; RT1 (min): 4.58; RT2 (min): 6.52; the first peak is product) to afford 353-13 (450 mg, 40.91%) as a yellow solid.

### 14. Synthesis of 353

The 353-13 (450 mg) was purified by Prep-HPLC with the following conditions (Column: CHIRALPAK IF, 2*25 cm, 5 μm; Mobile Phase A: Hex (0.5% 2M NH3-MeOH), Mobile Phase B: EtOH:DCM=1:1; Flow rate: 20 mL/min; Gradient: 30% B to 30% B in 12.5 min; Wave Length: 220/254 nm; RT1 (min): 8.657; RT2 (min): 9.447; the first peak is product) to afford 353 (53.4 mg, 11.80%) as a yellow solid.

LC-MS: (ES, m/z): [M+H]+ 569. H-NMR: (400 MHz, DMSO-d6, δ ppm): 0.81-0.93 (m, 4H), 1.37-1.79 (m, 7H), 1.83-1.95 (m, 3H), 2.28-2.30 (m, 2H), 2.74-2.84 (m, 2H), 3.15-3.32 (m, 3H), 3.42-3.44 (m, 5H), 4.27-4.30 (d, 1H), 4.47-4.50 (m, 1H), 7.01 (s, 1H), 7.19-7.21 (d, 1H), 7.31 (s, 1H), 7.42-7.46 (m, 1H), 7.65-7.70 (m, 2H), 7.75 (s, 1H), 8.32 (s, 1H).

### Example 354. Synthesis of Compound 354

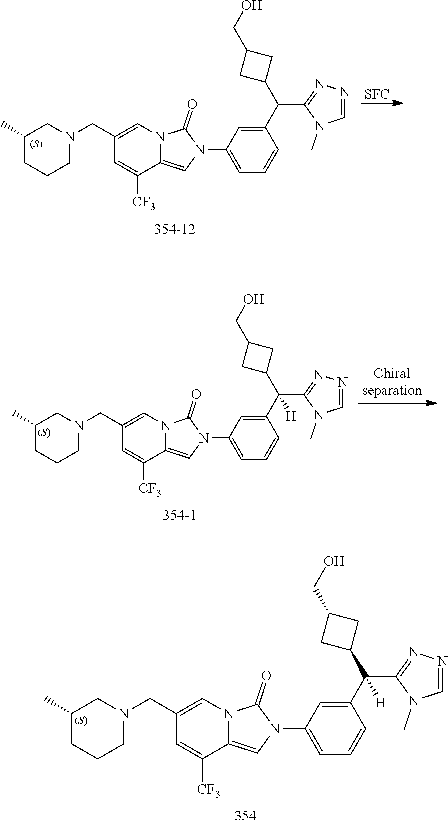

### 1. Synthesis of 354-1

The 354-12 (1.1 g) was purified by Prep-SFC with the following conditions (Column: CHIRAL ART Cellulose-SB, 3*25 cm, 5 μm; Mobile Phase A: CO2, Mobile Phase B: IPA (0.5% 2M NH3-MeOH); Flow rate: 100 mL/min; Gradient: isocratic 40% B; Column Temperature (° C.): 35; Back Pressure (bar): 100; Wave Length: 220 m; RT1 (min): 4.58; RT2 (min): 6.52; the second peak is product) to afford 354-1 (450 mg, 40.91%) as a yellow solid.

### 2. Synthesis of 354

The 354-1 (450 mg) was purified by Prep-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 30% B to 30% B in 13 min; Wave Length: 220/254 nm; RT1 (min): 8.187; RT2 (min): 11.097, the first peak is product) to afford 354 (240.2 mg, 5199%) as a yellow solid.

LC-MS: (ES, m/z): [M+H]+ 569. H-NMR-354 (400 MHz, DMSO-d6, δ ppm): 0.81-0.93 (m, 4H), 1.39-1.48 (m, 2H), 1.48-1.67 (m, 5H), 1.69-1.83 (m, 1H), 1.83-1.95 (m, 1H), 2.08-2.14 (m, 1H), 2.14-2.23 (m, 1H), 2.74-2.84 (m, 2H), 3.01-3.12 (m, 1H), 3.24 (s, 2H), 3.32 (s, 2H), 3.42 (s, 3H), 4.14-4.17 (d, 1H), 4.40-4.42 (m, 1H), 7.01 (s, 1H), 7.17-7.19 (d, 1H), 7.32 (s, 1H), 7.42-7.46 (m, 1H), 7.65-7.73 (m, 3H), 8.33 (s, 1H).

### Example 355. Synthesis of Compound 355

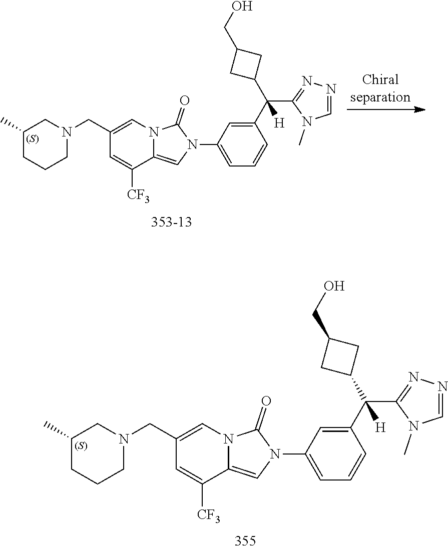

### 1. Synthesis of 355

The 353-13 (450 mg) was purified by Prep-HPLC with the following conditions (Column: CHIRALPAK IF, 2*25 cm, 5 μm; Mobile Phase A: Hex (0.5% 2M NH3-MeOH), Mobile Phase B: EtOH:DCM=1:1; Flow rate: 20 mL/min; Gradient: 30% B to 30% B in 12.5 min; Wave Length: 220/254 am; RT (min): 8.657; RT2 (min): 9,447; the second peak is product) to afford 355 (229.6 mg, 50.77%) as a yellow solid.

LC-MS: (ES, m/z): [M+H]+ 569. H-NMR-355 (400 MHz, DMSO-d6, δ ppm): 0.81-0.93 (m, 4H), 1.35-1.69 (m, 7H), 1.69-1.83 (m, 1H), 1.83-1.95 (m, 1H), 2.08-2.13 (m, 1H), 2.13-2.19 (m, 1H), 2.74-2.84 (m, 2H), 3.01-3.12 (m, 1H), 3.24 (s, 2H), 3.32 (s, 2H), 3.42 (s, 3H), 4.14-4.17 (d, 1H), 4.40-4.42 (m, 1H), 7.01 (s, 1H), 7.17-7.19 (d, 1H), 7.31 (s, 1H), 7.42-7.46 (m, 1H), 7.65-7.73 (m, 3H), 8.32 (s, 1H).

### Example 356. Synthesis of Compound 356

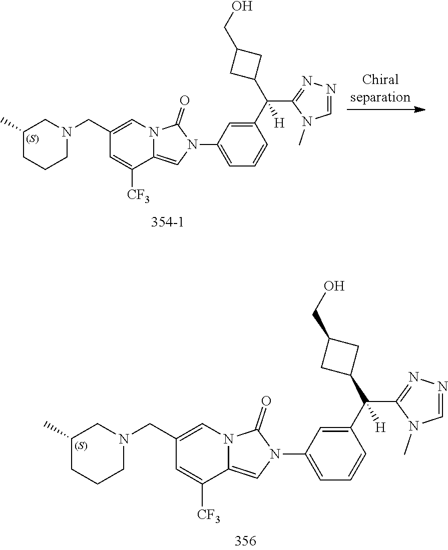

### 1. Synthesis of 356

The 354-1 (450 mg) was purified by Prep-HPLC with the following conditions (Column: CHIRALPAK IG, 2*25 cm, 5 n; Mobile Phase A: Hex (0.5% 2M NH3-MeOH), Mobile Phase B: EtOH:DCM=1:1; Flow rate: 20 mL/min; Gradient 30% B to 30% B in 13 min; Wave Length: 220/254 nm; RT1 (min): 8.187; RT2 (min): 11,097, the second peak is product) to afford 356 (51.6 mg, 11.29%) as a yellow solid.

LC-MS: (ES, m/z): [M+H]+ 569. H-NMR: (400 MHz, DMSO-d6, δ ppm): 0.81-0.93 (m, 4H), 1.37-1.79 (m, 7H), 1.83-1.95 (m, 3H), 2.28-2.35 (m, 2H), 2.65-2.3.01 (m, 2H), 3.15-3.17 (m, 1H), 3.32 (s, 2H), 3.34-3.44 (m, 5H), 4.27-4.30 (d, 1H), 4.48-4.51 (m, 1H), 7.07 (s, 1H), 7.20-7.22 (d, 1H), 7.33 (s, 1H), 7.42-7.46 (m, 1H), 7.68-7.75 (m, 3H), 8.32 (s, 1H).

### Example 357. Synthesis of Compound 357

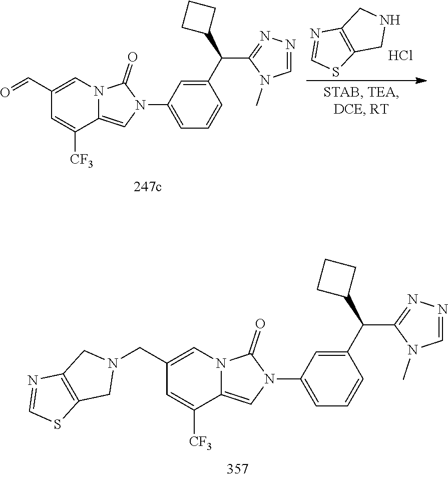

### 1. Synthesis of 357

To a stirred solution of 247 (300 mg, 0.659 mm ol, 1.00 equiv) and 4H,5H,6H-pyrrolo[3,4-d][1,3]thiazole dihydrochloride (262.28 mg, 1.318 mmol, 2 equiv) in DCE (5 mL) were added TEA (266.62 mg, 2.636 mmol, 4 equiv) at room temperature. The mixture was stirred for 30 min at room temperature. To the above mixture was added STAB (279.21 mg, 1.318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×20 mL). The resulting mixture was concentrated under reduced pressure. The crude product (130 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 27% B to 55% B in 8 min, 55% B; Wave Length: 220 nm; RT1 (min): 7.55) to afford 357 (117.5 mg, 31.00%) as a yellow solid.

LC-MS: (ES, m/z): [M+H]+: 566. H-NMR: (400 MHz, DMSO-d6, ppm): δ 1.80-1.82 (m, 5H), δ 2.01-2.16 (m, 1H), δ 3.15-3.21 (m, 1H), δ 3.33 (s, 3H), δ 3.78 (s, 2H), δ 3.91-3.93 (m, 2H), δ 4.02-4.03 (d, 2H), δ 4.25-4.28 (d, 1H), δ 7.11 (s, 1H), δ 7.09-7.21 (d, 1H), δ 7.33 (s, 1H), δ 7.43-7.46 (m, 1H), δ 7.69-7.74 (m, 2H), δ 7.80 (s, 1H), δ 8.33 (s, 1H), δ 9.00 (s, 1H).

### Example 358. Synthesis of Compound 358

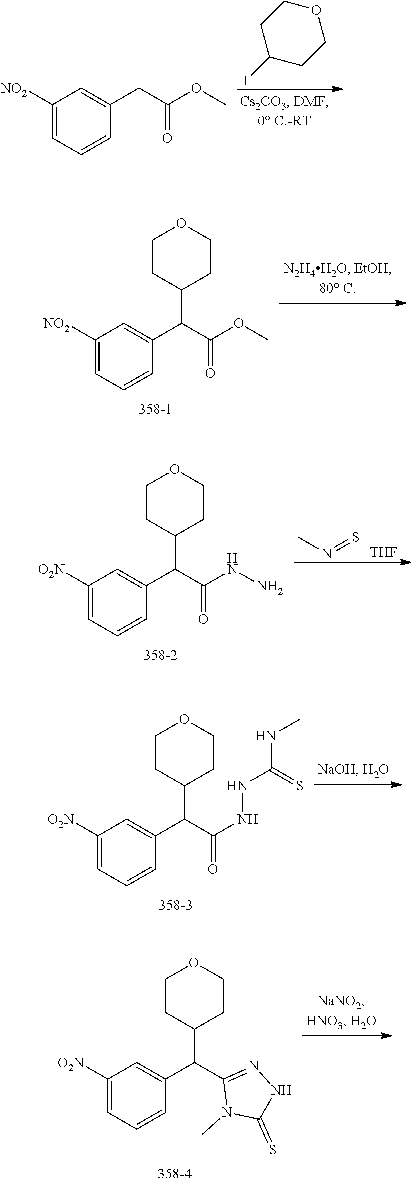

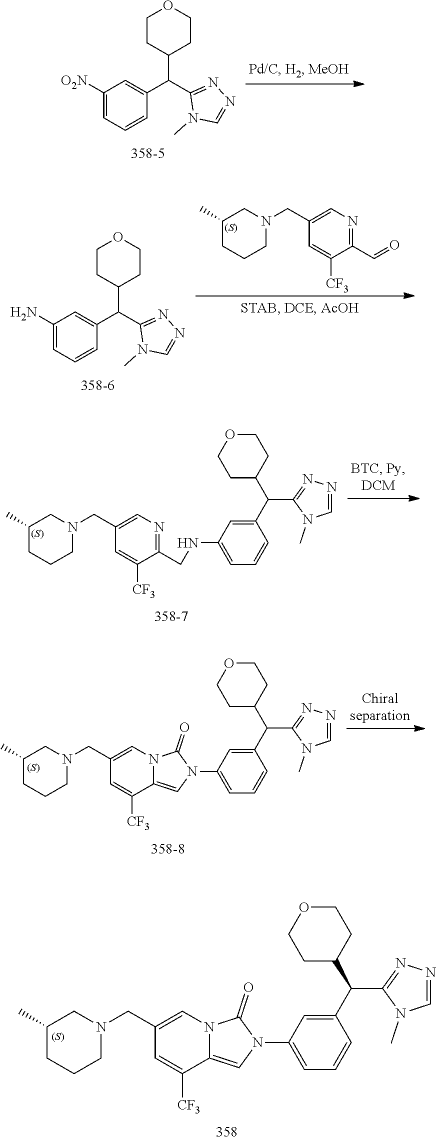

### 1. Synthesis of 358-1

Into a 250 mL 3-necked round-bottom flask were added methyl,2-(3-nitrophenyl) acetate (10 g, 51.236 mmol, 1 equiv), 4-iodooxane (32.59 g, 153.708 mmol, 3 equiv), DMF (100 mL) and Cs2CO3 (83.47 g, 256.180 mmol, 5 equiv) at 0° C. The resulting mixture was stirred for overnight at room temperature under nitrogen atmosphere. The reaction was quenched with sat. NH4Cl (aq.) (200 ml) at room temperature. The aqueous layer was extracted with EtOAc (3×200 ml). The resulting mixture was concentrated under reduced pressure. The residue was purified by silica gel column chromatography, eluted with PE/EA (10:1) to afford 358-1 (13 g, 89.03%) as a white solid.

### 2. Synthesis of 358-2

Into a 500 mL 3-necked round-bottom flask were added 358-1 (13 g, 46.546 mmol, 1 equiv), NH2NH2H2O (69.90 g, 1396.380 mmol, 30 equiv) and EtOH (130 mL) at room temperature. The resulting mixture was stirred for overnight at 80° C. The reaction was quenched with sat. NH4Cl (aq.) (200 mL) at room temperature. The aqueous layer was extracted with EtOAc (3×200 mL). The resulting mixture was concentrated under reduced pressure to afford 358-2 (10.5 g, 76.73%) as a white solid.

### 3. Synthesis of 358-3

Into a 250 mL 3-necked round-bottom flask were added 358-2 (10.5 g, 37.595 mmol, 1 equiv), methyl isothiocyanate (6.87 g, 93.987 mmol, 2.5 equiv) and tetrahydrofuran (110 mL) at room temperature. The mixture was stirred for overnight at room temperature. The reaction was quenched with sat. NH4Cl (aq.) (200 ml) at room temperature. The aqueous layer was extracted with EtOAc (3×200 mL). The resulting mixture was concentrated under reduced pressure. The residue was purified by silica gel column chromatography, eluted with PE/EA (8:1) to afford 358-3 (12 g, 86.05%) as a white solid.

### 4. Synthesis of 358-4

A mixture of 358-3 (10 g, 28.376 mmol, 1 equiv) in sodium hydroxide (280 ml, 1 M) was stirred for overnight at room temperature. The reaction was dilated with water. And then the pH value of the solution was adjusted to 5 with HCL aq. (1 M). The precipitated solids were collected by filtration and the resulting mixture was concentrated under reduced pressure to afford the title compound 358-4 as a white solid, which was used without purification.

### 5. Synthesis of 358-5

To a stirred solution of 358-5 (9 g, 26915 mmol, 1 equiv) in water (48 ml) was added NaNO2 (18.57 g, 269.150 mmol, 10 equiv). This was followed by addition of HNO3 (269.15 ml, 1 M) dropwise with stirring at 0° C. The mixture was stirred for overnight at room temperature. And then the pH value of the solution was adjusted to 8 with sat. NaHCO3 (aq.) at room temperature. The aqueous layer was extracted with EtOAc (3×300 mL). The resulting mixture was concentrated under reduced pressure. The residue was purified by silica gel column chromatography, eluted with PE/EA (5:1) to afford 358-6 (8 g, 92.41%) as a yellow oil.

### 6. Synthesis of 358-6

To a solution of 358-5 (2 g, 6.615 mmol, 1 equiv) in 20 mL MeOH was added Pd/C (10%, 0.21 g) under nitrogen atmosphere in a 100 mL round-bottom flask. The mixture was hydrogenated at room temperature for 5 h under hydrogen atmosphere using a hydrogen balloon, filtered through a Celite pad and the mixture was concentrated under reduced pressure. This resulted in 358-6 (1.8 g, 93.91%) as a yellow oil.

### 7. Synthesis of 358-7

Into a 100 mL round-bottom flask were added 358-6 (1 g, 3.672 mmol, 1 equiv), DCE (10 mL), I-2 (1.37 g, 4,774 mmol, 1.3 equiv) and STAB (2.33 g, 11.016 mmol, 3 equiv) at room temperature. The resulting mixture was stirred for overnight at room temperature. The reaction was quenched with sat. NH4Cl (aq.) (30 ml) at room temperature. The aqueous layer was extracted with CH2Cl2 (3×30 mL) and the mixture was concentrated under reduced pressure. The residue was purified by Prep-TLC (CH2Cl2/MeOH 15:1) to afford 358-7 (1.5 g, 70.01%) as a yellow oil.

### 8. Synthesis of 358-8

Into a 100 mL round-bottom flask were added 358-7 (1.5 g, 2,764 mmol, 1 equiv), DCM (30 mL) and pyridine (1.31 g, 16.584 mmol, 6 equiv) at room temperature. To the above mixture was added triphosgene (0.41 g, 1.382 mmol, 0.5 equiv) at 0° C. The resulting mixture was stirred for 5 min at 0° C. The reaction was quenched with sat. NaHCO₃ (aq.) (50 mL) at room temperature. The aqueous layer was extracted with CH2Cl2 (3×40 mL). The resulting mixture was concentrated under vacuum. The residue was purified by Prep-TLC (CH2Cl2/MeOH 10:1) to afford 358-8 (1 g, 61.71%) as a yellow solid.

### 9. Synthesis of 358-0

The 358-9 (250 mg) was purified by Chiral separation with the following conditions Column: CHIRALPAK IH, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 20% B to 20% B in 10.5 min; Wave Length: 220/254 nm; RT1 (min): 6.381; RT2 (min): 8.334; The second peak was the product. Sample Solvent: EtOH:DCM=1:1-HPLC; Injection Volume: 1 mL; Number of Runs: 7) to afford 358 (105.4 mg, 42.16%) as a yellow solid.

LC-MS: (ES, m/z): [M+H]+ 569. H-NMR: (400 MHz, DMSO, δ ppm): 0.72-0.99 (d, 4H), 1.11-1.37 (m, 3H), 1.37-1.53 (m, 1H), 1.53-1.71 (m, 5H), 1.80-1.99 (m, 1H), 2.69-2.88 (d, 2H), 3.15-3.31 (m, 5H), 3.45-3.58 (s, 3H), 3.72-3.88 (m, 2H), 4.02-4.15 (d, 1H), 6.91-7.06 (s, 1H), 7.23-7.36 (d, 2H), 7.38-7.51 (m, 1H), 7.60-7.66 (s, 1H), 7.66-7.75 (d, 1H), 7.76-7.86 (s, 1H), 8.26-8.38 (s, 1H).

### Example 359. Synthesis of Compound 359

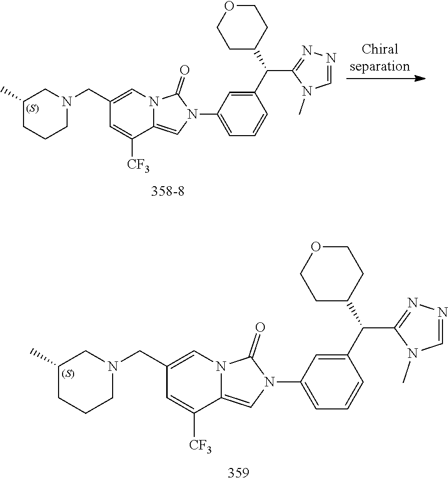

### 1. Synthesis of 359

The 358-8 (250 mg) was purified by Chiral separation with the following conditions Column: CHIRALPAK TH, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 20% B to 20% B in 10.5 min; Wave Length: 220/254 nm; RT1 (min): 6,381; RT2 (min): 8.334; The first peak was the product. Sample Solvent: EtOH:DCM=1:1-HPLC; Injection Volume: 1 mL; Number of Runs: 7) to afford 359 (112.6 mg, 45.04%) as a yellow solid.

LC-MS: (ES, m/z): [M+H]+ 569. H-NMR: (400 MHz, DMSO, δ ppm): 0.72-0.99 (d, 4H), 1.11-1.37 (m, 3H), 1.37-1.53 (m, 1H), 1.53-1.71 (m, 5H), 1.80-1.99 (m, 1H), 2.69-2.88 (d, 2H), 3.15-3.31 (m, 5H), 3.45-3.58 (s, 3H), 3.72-3.88 (m, 2H), 4.02-4.15 (d, 1H), 6.91-7.06 (s, 1H), 7.23-7.36 (d, 2H), 7.38-7.51 (m, 1H), 7.60-7.66 (s, 1H), 7.66-7.75 (d, 1H), 7.76-7.86 (s, 1H), 8.26-8.38 (s, 1H).

### Example 360. Synthesis of Compound 360

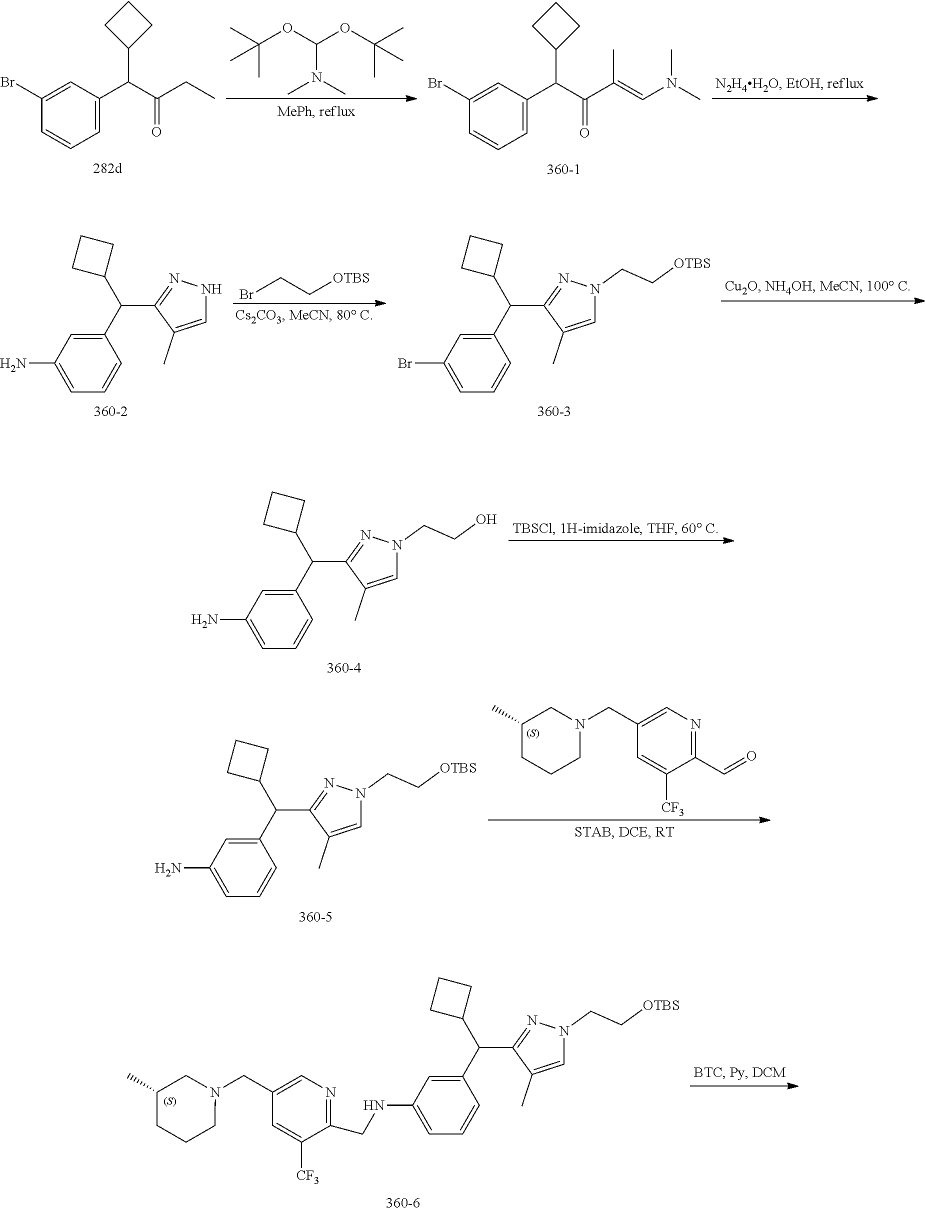

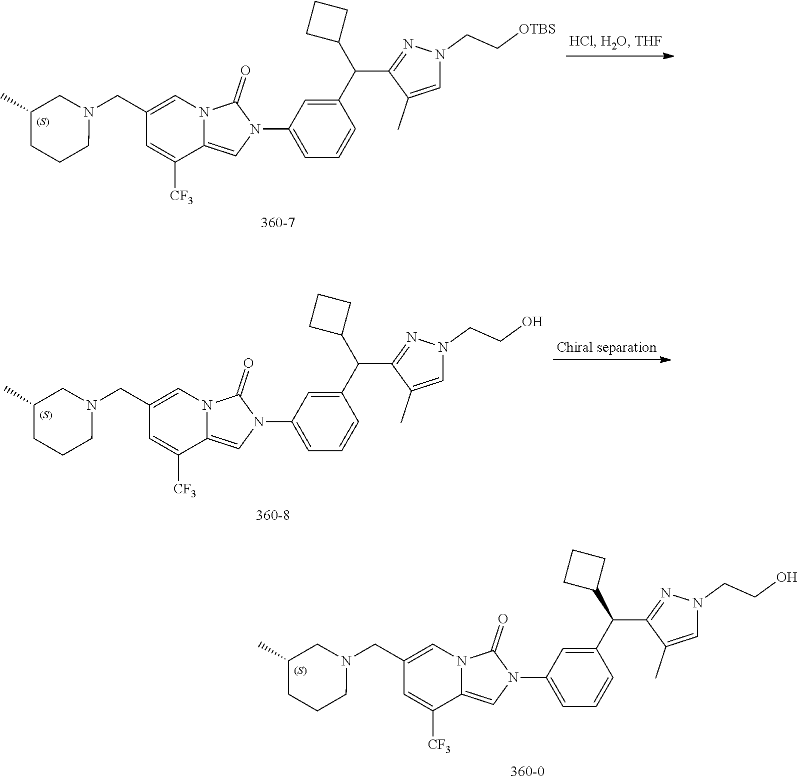

### 1. Synthesis of 360-1

To a stirred solution of 282-4 (15 g, 53.344 mmol, 1 equiv) in Toluene (150 mL) was added [bis(tert-butoxy)methyl]dimethylamine (54.23 g, 266,720 mmol, 5 equiv) at room temperature. The resulting mixture was stirred overnight at 110° C. The resulting mixture was concentrated under vacuum. The residue was purified by silica gel column chromatography, eluted with PE/EA (50:1) to afford 360-1 (9 g, 30.10%) as a yellow oil.

### 2. Synthesis of 360-2

To a stirred solution of 360-1 (9 g, 26,764 mmol, 1 equiv) in EtOH (100 mL) was added hydrazine hydrate (98%)(13.40 g, 267.640 mmol, 10 equiv) at room temperature. The resulting mixture was stirred for 6 h at 80° C. The resulting mixture was diluted with water (300 mL). The aqueous layer was extracted with EtOAc (2×100 mL). The resulting mixture was concentrated under vacuum. The residue was purified by reverse flash chromatography with the following conditions (Column, C18 silica gel; Mobile Phase A: Water (0.05% FA), Mobile Phase B: ACN; Flow rate: 100 mL/min; Gradient: 10% B to 50% B in 20 min, Wave Length: 220 nm) to afford the crude product. The crude product (9 g) was purified by HP-FLASH with the following conditions (Column: Welch Utimate AQ-C18, 50*250 mm*10 μm; Mobile Phase A: Water (0.05% TEA), Mobile Phase B: ACN; Flow rate: 100 mL/min; Gradient: 35% B to 65% B in 30 min, Wave Length: 220 nm) to afford 360-2 (4.1 g, 46.68%) as a light yellow oil.

### 3. Synthesis of 360-3

To a stirred solution of 360-2 (2 g, 6.553 mmol, 1 equiv) and (2-bromoethoxy)(tert-butyl)dimethylsilane (3.92 g, 16.383 mmol, 2.5 equiv) in MeCN (100 mL) was added Cs2CO3 (6.40 g, 19.659 mmol, 3 equiv) at room temperature. The resulting mixture was stirred overnight at 80° C. The resulting mixture was diluted with water (200 mL). The aqueous layer was extracted with EtOAc (2×100 ml). The resulting mixture was concentrated under vacuum. The residue was purified by Prep-TLC (PE/EA 5:1) to afford 360-4 (2.2 g, 67.36%) as a colorless oil.

### 4. Synthesis of 360-4

To a solution of 360-3 (2 g, 4.315 mmol, 1 equiv) and Cu2O (0.12 g 0.863 mmol, 0.2 equiv) in MeCN (10 mL) was added NH40H (10 mL) in a pressure tank. The resulting mixture was stirred for overnight at 100° C. The mixture was allowed to cool down to room temperature. The resulting mixture was diluted with water (100 mL). The resulting mixture was extracted with EtOAc (3×100 mL). The combined organic layers were dried over anhydrous Na2SO4. After filtration, the filtrate was concentrated under reduced pressure. The crude product was used in the next step directly without further purification. This resulted in 360-3 (1.2 g, 87.71%) as a light yellow oil.

### 5. Synthesis of 360-5

To a stirred solution of 360-4 (1.2 g, 4,205 mmol, 1 equiv) and t-butyldimethylchlorosilane (1.90 g, 12.615 mmol, 3 equiv) in tetrahydrofuran (100 mL) was added 1H-imidazole (1.43 g, 21.025 mmol, 5 equiv) at room temperature. The resulting mixture was stirred for 4 h at 60° C. The reaction was quenched with water (100 mL) at room temperature. The aqueous layer was extracted with EtOAc (2×100 mL). The resulting mixture was concentrated under vacuum. The residue was purified by Prep-TLC (PE/EA 8:1) to afford 360-5 (1.2 g, 69.98%) as a light yellow oil.

### 6. Synthesis of 360-6

To a stirred solution of 360-5 (0.8 g, 2.002 mmol, 1 equiv) and I-2 (0.75 g 2.603 mmol, 1.3 equiv) in DCE (10 mL) was added STAB (0.85 g, 4.004 mmol, 2 equiv) at room temperature. The resulting mixture was stirred for 4 h at room temperature. The reaction was quenched with saturated NaHCO3 (aq.) (100 mL) at room temperature. The aqueous layer was extracted with DCM (2×100 mL). The residue was purified by Prep-TLC (PE/EA 2:1) to afford 360-6 (1.2 g, 87.69%) as a light yellow solid.

### 7. Synthesis of 360-7

To a stirred solution of 360-6 (1.2 g, 1.791 mmol, 1 equiv) and Pyridine (1.42 g, 17.910 mmol, 10 equiv) in DCM (20 mL) was added Triphosgene (0.21 g, 0.716 mmol 0.4 equiv) at room temperature. The resulting mixture was stirred for 10 min at room temperature. The reaction was quenched with saturated NaHCO3 (aq.) (100 mL) at room temperature. The aqueous layer was extracted with DCM (2×50 mL). The resulting mixture was concentrated under vacuum. The crude product was used in the next step directly without further purification. This resulted in 360-6 (1 g, 74.61%) as a yellow solid.

### 8. Synthesis of 360-8

To a stirred solution of 360-7 (1 g, 1.437 mmol, 1 equiv) in THF (10 mL) was added HCl (aq.)(10 mL, 1M) at room temperature. The resulting mixture was stirred for 3 h at room temperature. The mixture was neutralized to pH 7 with saturated NaHCO3 (aq.). The aqueous layer was extracted with DCM (2×100 mL). The resulting mixture was concentrated under vacuum. The residue was purified by Prep-TLC (DCM/MeOH 20:1) to afford 360-8 (700 mg, 80.40%) as a yellow solid.

### 9. Synthesis of 360

The 360-8 (400 mg) was purified by Prep-Chiral-HPLC with the following conditions (Column: CHIRALPAK IF, 2*25 cm, 5 μm; Mobile Phase A: Hex (0.5% 2M NH3-MeOH), Mobile Phase B: EtOH:DCM=1:1; Flow rate: 20 mL/min; Gradient: 10% B to 10% B in 10.5 min; Wave Length: 220/254 nm; RT1 (min): 5315; RT2 (min): 7.05; the second peak is product) to afford 360 (155.5 mg, 38.56%) as a yellow solid.

LC-MS: (ES, m/z): [M+H]+ 582. H-NMR: (400 MHz, DMSO-d6, δ ppm): 0.82-0.93 (m, 4H), 1.43-1.53 (m, 1H), 1.53-1.71 (m, 5H), 1.71-1.80 (m, 4H), 1.80-1.95 (m, 4H), 1.95-2.02 (m, 1H), 2.71-2.85 (m, 2H), 3.09-3.12 (m, 1H), 3.64-3.68 (m, 2H), 3.92-3.95 (d, 1H), 4.01-4.03 (m, 2H), 7.00 (s, 1H), 7.21-7.26 (m, 2H), 7.32 (s, 1H), 7.35-7.39 (m, 1H), 7.58-7.60 (d, 1H), 7.66 (s, 1H), 7.73 (s, 1H).

### Example 361. Synthesis of Compound 361

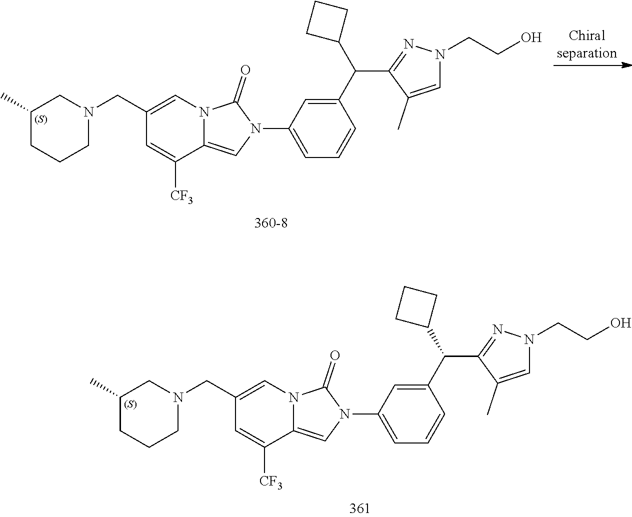

### 1. Synthesis of 361

The 360-8 (400 mg) was purified by Prep-Chiral-HPLC with the following conditions (Column: CHIRALPAK IF, 2*25 cm, 5 μm; Mobile Phase A: Hex (0.5% 2M NH3-MeOH), Mobile Phase B: EtOH:DCM=1:1; Flow rate: 20 mL/min; Gradient: 10% B to 10% B in 10.5 min; Wave Length: 220/254 n; RT1 (min): 5.715; RT2 (min): 7.05; the first peak is product) to afford 361 (166.2 mg, 41.13%) as a yellow solid.

LC-MS-361: (ES, m/z): [M+H]+ 582

H-NMR-361: (400 MHz, DMSO-d6, δ ppm): 0.82-0.93 (m, 4H), 1.43-1.53 (m, 1H), 1.53-1.71 (m, 5H), 1.71-1.80 (m, 4H), 1.80-2.02 (m, 5H), 2.71-2.85 (m, 2H), 3.09-3.12 (m, 1H), 3.64-3.68 (m, 2H), 3.92-3.95 (d, 1H), 4.01-4.03 (m, 2H), 7.00 (s, 1H), 7.21-7.26 (m, 2H), 7.32 (s, 1H), 7.35-7.39 (m, 1H), 7.58-7.60 (d, 1H), 7.66 (s, 1H), 7.73 (s, 1H).

### Example 362. Synthesis of Compound 362_P1 and 362_P2

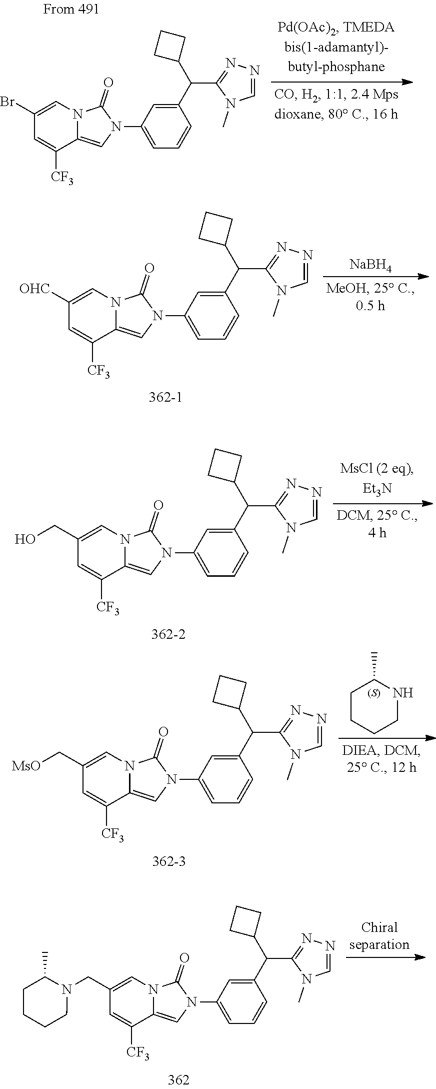

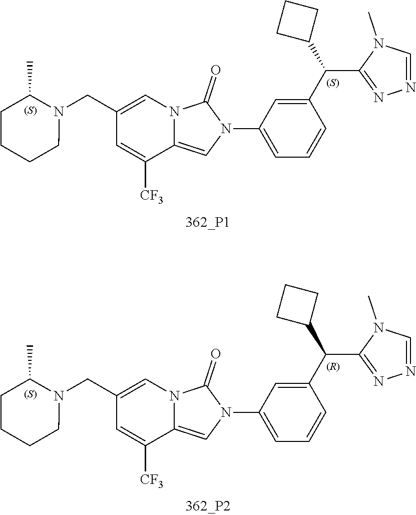

### 1. Synthesis of 362-1

To a solution of 6-bromo-2-[3-[cyclobutyl-(4-methyl-1,2,4-triazol-3-yl)methyl]phenyl]-8-(trifluoromethyl)imidazo[1,5-a]pyridin-3-one (1 g, 1.98 mmol, 1 equiv) in dioxane (40 mL) was added N,N,N′,N′-tetramethylethane-1,2-diamine (459.02 mg, 3.95 mmol, 2 equiv) and diacetoxypalladium (88.68 mg, 395.01 umol, 0.2 equiv), bis(1-adamantyl)-butyl-phosphane (141.63 ag, 395.01 umol, 0.2 equiv). The mixture was stirred at 80° C. for 16 hr under CO/H2 (1:1) (2.5 Mpa). The reaction was poured into water (50 mL) and the resulting mixture was extracted with EtOAc (2×50 mL). The organic phase was washed with sat Na1HCO3 (20 mL), brine (20 mL), dried over anhydrous Na2SO4, concentrated in vacuum to afford 362-1 (0.65 g, crude) as a light yellow solid.

### 2. Synthesis of 362-2

To a solution of 362-1 (3 g, 6.59 mmol, 1 equiv) in MeOH (40 mL) was added NaBH4 (747.62 mg, 19.76 mmol, 3 equiv). The mixture was stirred at 25° C. for 0.5 hr. The reaction was poured into water (50 mL) and the resulting mixture was concentrated in vacuum to remove MeOH, then extracted with EtOAc (2×30 mL). The organic phase was washed with brine (10 mL), dried over anhydrous Na2SO4, concentrated in vacuum to give a residue 362-2 (3 g, crude) as a yellow solid.

### 3. Synthesis of 362-3

To a solution of 362-2 (300 mg, 655.81 umol, 1 equiv) in DCM (10 mL) was added Et3N (199.08 mg, 1.97 mmol, 273.84 uL, 3 equiv) and MsCl (150.25 mg, 1.31 mmol, 2 equiv). The mixture was stirred at 25° C. for 1 hr. The reaction was poured into water (30 mL) and the resulting mixture was extracted with DCM (2×30 mL). The organic phase was washed with brine (10 mL), dried over anhydrous Na2SO4, concentrated in vacuum to give a residue 362-3 (330 mg, crude) as a black solid.

### 4. Synthesis of 362

To a solution of 362-3 (270 mg 504.17 umol, 1 equiv) in DCM (9 mL) was added Et3N (153 mg, 1.51 mmol, 210.52 uL, 3 equiv) and (2S)-2-methylpiperidine (100 mg, 1.01 mmol, 119.19 uL, 2 equiv). The resulting mixture was stirred at 25° C. for 12 hrs. The reaction was poured into water (30 mL) and the resulting mixture was extracted with DCM (2×30 mL). The organic phase was washed with brine (10 mL), dried over anhydrous Na2SO4, concentrated in vacuum to give a residue. The residue was purified by prep-HPLC (column: Waters Xbridge BEH C18 100*30 mm*10 um; mobile phase: [water (NH4HCO3)-ACN]; B %: 40%-70%, 10 min) to afford 362 (22 mg, 8%) as a light yellow solid,

### 5. Synthesis of 362_P1&P2

362 (22 mg) was purified by Chiral separation with the following conditions (column: REGIS WHELK-O1 (250 mm*25 mm, 10 um); Mobile Phase: [Neu-ETOH]; B %: 55%-55%, 12 min, Flow rate: 70 mL/min; Wave Length: 220/254 nm; RT1 (min): 3.47; RT2 (min): 4.77) to afford 362_P1 (6.5 mg, 28% yield) as a yellow solid.

MS-362_P1: (ES, m/z): [M+H]+ 538.2

1H-NMR-362_P1: (400 MHz, DMSO-d6) δ ppm 8.33 (s, 1H), 7.75 (s, 1H), 7.69-7.60 (m, 2H), 7.47-7.43 (m, 1H), 7.31 (m, 1H), 7.21-7.19 (d, J=8 Hz, 1H), 6.99 (s, 1H), 4.31-4.20 (m, 1H), 3.70-3.67 (d, J=12 Hz, 1H), 3.44 (s, 3H), 3.10-2.99 (m, 1H), 3.16-3.14 (m, 1H), 2.88-2.74 (m, 1H), 2.43-2.40 (m, 2H), 2.19-2.14 (m, 2H), 1.78-1.67 (m, 4H), 1.61-1.59 (m, 3H), 1.50-1.38 (m, 3H), 1.16-1.11 (m, 3H) 362_P2 (5.6 mg, 24% yield) as a yellow solid.

MS-362-P2: (ES, m/z): [M+H]+ 538.2. 1H-NMR-362_P2: (400 MHz, DMSO-d6) δ ppm 8.33 (s, 1H), 7.74 (s, 1H), 7.71-7.66 (m, 2H), 7.46-7.42 (m, 1H), 7.31 (s, 1H), 7.19 (d, J=8 Hz, 1H), 6.98 (s, 1H), 4.28-4.21 (m, 1H), 3.69-3.66 (d, J=12 Hz, 1H), 3.43 (s 3H), 3.28-3.25 (m, 1H), 3.04-3.00 (d, J=12 Hz, 1H), 2.79-2.71 (m, 1H), 2.43-2.33 (m, 2H), 2.04-2.03 (m, 1H), 2.02-2.01 (m, 1H), 1.76-1.74 (m, 4H), 1.64-1.62 (m, 3H), 1.49-1.44 (m, 3H), 1.05-1.01 (m, 3H).

### Example 363. Synthesis of Compound 363_P1 and 363_P2

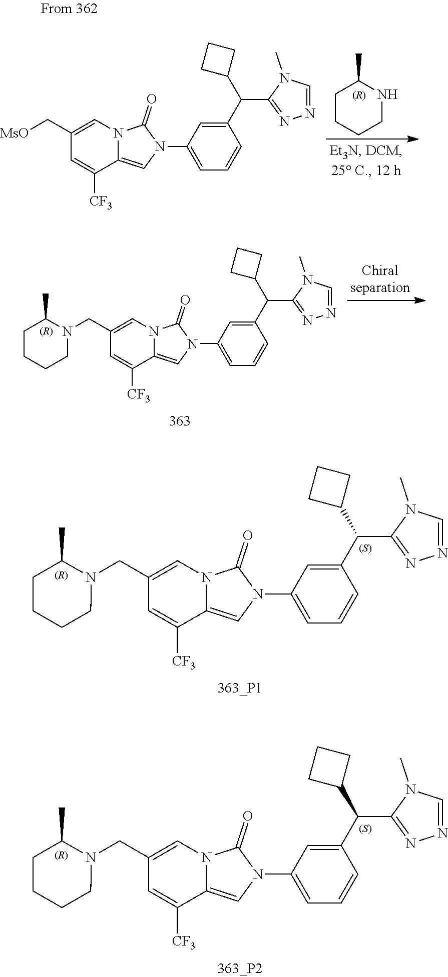

### 1. Synthesis of 363

To a solution of [2-[3-[cyclobutyl-(4-methyl-1,2,4-triazol-3-yl)methyl]phenyl]-3-oxo-8-(trifluoromethyl)imidazo[1,5-a]pyridin-6-yl]methyl methanesulfonate (200 mg, 373.46 umol, 1.0 equiv) in DCM (5 mL) was added Et3N (113.3 mg, 1.12 mmol, 3 equiv) and (2R)-2-methylpiperidine (74.07 mg, 746.91 umol, 2 equiv). The mixture was stirred at 25° C. for 12 hr. The reaction was poured into water (30 mL) and the resulting mixture was extracted with DCM (2×30 ml). The organic phase was washed with brine (10 mL), dried over anhydrous Na2SO4, concentrated in vacuum to give a residue. The residue was purified by prep-HPLC(Column: Waters Xbridge Prep OBD C18 150*40 mm*10 um; Mobile phase A: [water (NH4HCO3)-ACN]; R %: ACN: Flow rate: 50 mL/min: Gradient: 45% B to 75% B in 8 min. Wave Length: 220 nm; RT1 (min): 5.1) to afford 363 (60 mg, 29% yield) as a yellow solid.

### 2. Synthesis of 363_P1& P2

The 363 (60 mg) was purified by Chiral separation with the following conditions (column: DAICEL CHIRALCEL OD (250 mm*30 mm, 10 um); Mobile Phase A %: [Neu-MeOH]; B %: 40%-40%, 8 min, Flow rate: 70 mL/min; Wave Length: 220/254 nm; RT1 (min): 130; RT2 (min): 1.38) to afford 363_P1 (20.0 mg, 33% yield) as a yellow solid.

MS-363_P1 (ES, m/z): [M+H]+ 539.2. 1H-NMR-363_P1 (400 MHz, DMSO-d6) δ=8.33 (s, 1H), 7.75 (s, 1H), 7.73-7.71 (m, 1H), 7.71-7.69 (m, 1H), 7.45-7.38 (m, 1H), 7.20-7.13 (m, 1H), 7.00-6.91 (m, 1H), 6.79-6.72 (m, 1H), 4.26-4.25 (m, 1H), 3.67-3.44 (m, 1H), 3.43 (s, 3H), 3.41-3.33 (m, 2H), 3.05-3.01 (m, 1H), 2.83-2.68 (m, 1H), 2.47-2.45 (m, 1H), 2.25-2.16 (m, 2H), 1.77-1.75 (m, 4H), 1.70-1.68 (m, 2H), 1.50-1.43 (m, 1H), 1.40-1.37 (m, 1H), 1.28-1.25 (m, 2H), 1.10-1.08 (m, 3H)

363_P2 (25.5 mg, 41% yield) as a yellow solid.

363-P2: (ES, m/z): [M+H]+ 539.2. 1H-NMR-363-P2: (400 MHz, DMSO-d6) δ 8.33 (s, 1H), 7.75 (s, 1H), 7.73-7.71 (m, 1H), 7.71-7.69 (m, 1H), 7.45-7.38 (m, 1H), 7.20-7.13 (m, 1H), 7.00-6.91 (m, 1H), 6.79-6.72 (m, 1H), 4.26-4.25 (m, 1H), 3.67-3.44 (m, 1H), 3.50 (s, 3H), 3.41-3.33 (m, 2H), 3.05-3.01 (m, 1H), 2.83-2.68 (m, 1H), 2.47-2.45 (m, 1H), 2.25-2.16 (m, 2H), 1.77-1.75 (m, 4H), 1.70-1.68 (m, 2H), 1.50-1.43 (m, 1H), 1.40-1.37 (m, 1H), 1.28-1.25 (m, 2H), 1.10-1.08 (m, 3H).

### Example 364. Synthesis of Compound 364_P1 and 364_P2

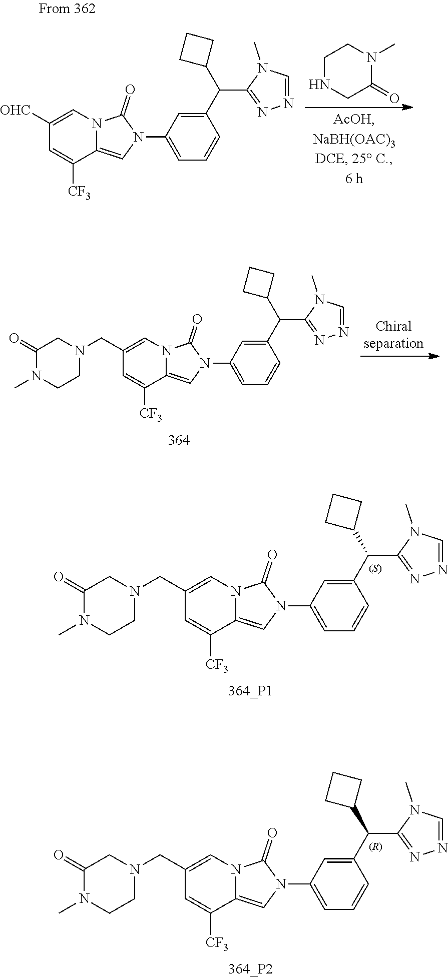

### 1. Synthesis of 364

To a solution of 2-[3-[cyclobutyl-(4-methyl-1,2,4-triazol-3-yl)methyl]phenyl]-3-oxo-8-(trifluoromethyl)imidazo[1,5-a]pyridine-6-carbaldehyde (400 mg, 878.29 umol, 1.0 equiv.), I-methylpiperazin-2-one (200 mg, 1.76 mmol, 2.0 equiv.) in DCE (10 mL) was added dropwise AcOH (52 mg, 878.29 umol, 50.23 uL, 1.0 equiv.) at 25° C. After addition, the mixture was stirred at this temperature for 2 hrs, and then NaBH(OAc)3 (558 mg, 2.63 mmol, 3.0 eq) was added dropwise. The resulting mixture was stirred at 25° C. for 14 hrs. The resulting mixture was stirred at 25° C. for 6 hrs. The reaction was poured into sat NaHCO₃ (30 mL) and the resulting mixture was extracted with DCM (2×20 mL). The organic phase was washed with brine (10 mL), dried over anhydrous Na2SO4, concentrated in vacuum to give a residue. The residue was purified by prep-HPLC (column: Waters Xbridge BEH C18 250*50 mm*10 um; mobile phase: [water (NH4HCO3)-ACN]; B %: 25%-55%, 10 min) to afford 364 (466 mg, 87%) as a light yellow solid.

### 2. Synthesis of 364_P1, P2

The 364 (70 mg) was purified by Chiral separation with the following conditions (column: DAICEL CHIRALCEL OD (250 mm*30 mm, 10 um); Mobile Phase: [Neu-IPA]; B %: 45%-45%, 7 min, Flow rate: 70 mL/min; Wave Length: 254 nm; RT1 (min): 1.54; RT2 (min): 1.69) to afford 364_P1 (17.2 mg, 23% yield) as a yellow solid.

MS-364-P1: (ES, m/z): [M+H]+ 553.2. 1H-NMR-364_P1: (400 MHz, CHLOROFORM-d) δ ppm 8.00 (s, 1H), 7.58-7.52 (m, 2H), 7.50-7.43 (m, 1H), 7.39-7.31 (m, 1H), 7.19-7.17 (d, J=8.0 Hz, 1H), 6.96 (s, 1H), 6.78 (s, 1H), 3.93-3.88 (m, 1H), 3.37 (s, 3H), 3.32-3.25 (m, 5H), 3.15-3.09 (m, 2H), 2.94 (s, 3H), 2.72-2.64 (m, 2H), 2.33-2.21 (m, 1H), 1.82-1.64 (m, 5H) 364_P2 (15.6 mg, 21% yield) as a yellow solid.

MS-364-P2: (ES, m/z): [M+H]+ 553.2. 1H-NMR-364_P2: (400 MHz, CHLOROFORM-d) δ ppm 8.05 (s, 1H) 7.66-7.60 (m, 2H) 7.59-7.53 (m, 1H) 7.47-7.40 (m, 1H) 7.21-7.17 (m, 1H) 6.94 (s, 1H) 6.79 (s, 1H), 3.99-3.96 (d, J=12 Hz, 1H) 3.32 (s, 3H) 3.38-3.33 (m, 5H), 3.24-3.15 (m, 2H), 2.95 (m, 3H), 2.80-2.71 (m, 2H) 2.41-2.29 (m, 1H), 2.00-1.79 (m, 5H).

### Example 365. Synthesis of Compound 365

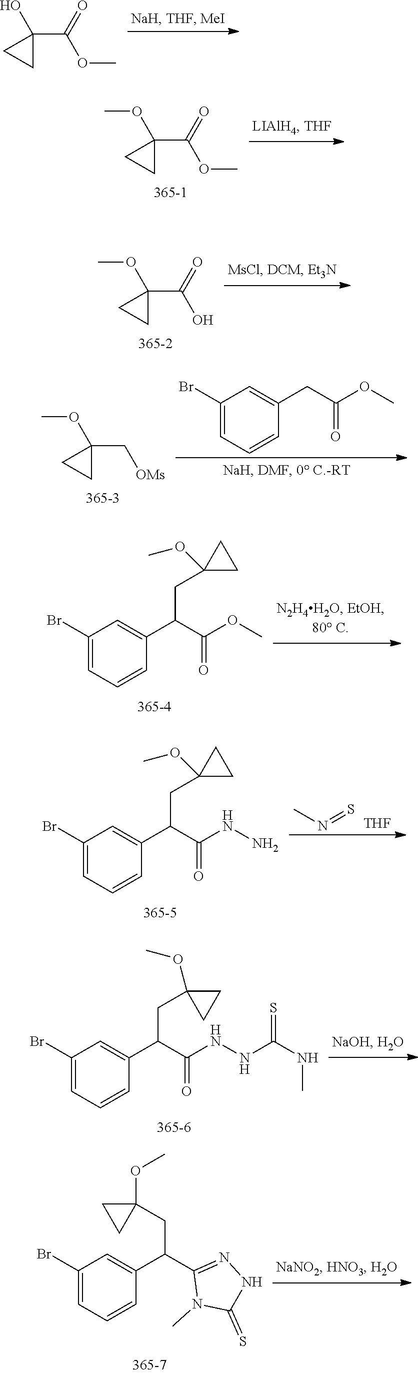

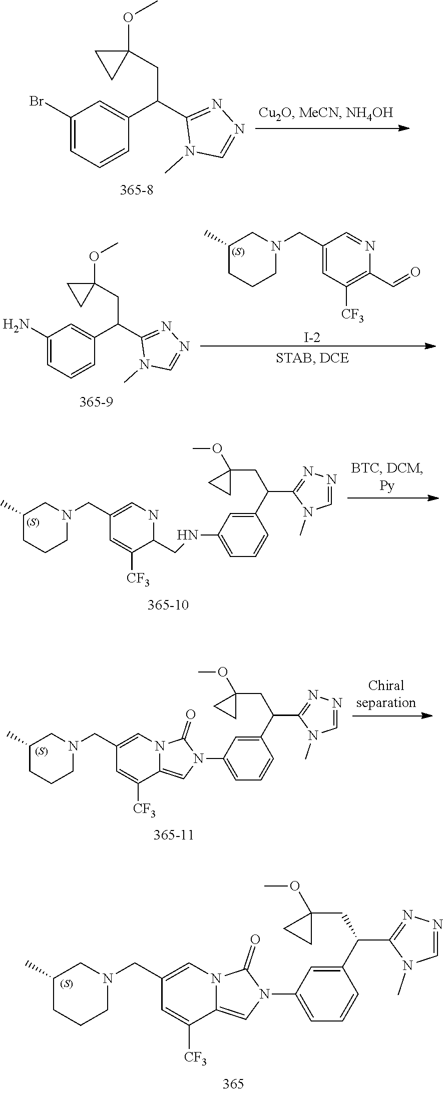

### 1. Synthesis of 365-1

To a stirred solution of methyl 1-hydroxycyclopropane-1-carboxylate (23 g, 198.078 mmol, 1 equiv) in THF (250 mL) was added NaI (11.88 g, 297.117 mmol, 1.5 equiv, 60%) in four portions at 0° C. under nitrogen atmosphere. The resulting mixture was stirred for 30 min at 0° C. under nitrogen atmosphere. To the above mixture was added MeI (50.61 g, 356.540 mmol, 1.8 equiv) dropwise over 20 min at ° C. The resulting mixture was stirred for additional overnight at room temperature. The reaction was quenched by the addition of sat. NH4Cl (aq.) (750 mL) at 0° C. The resulting mixture was extracted with EtOEt (2×300 mL). The combined organic layers were washed with brine (100 mL), dried over anhydrous Na2SO4. After filtration, the filtrate was concentrated under reduced pressure. This resulted in 365-1 (25 g, 96.98%) as a light yellow oil.

### 2. Synthesis of 365-2

To a stirred mixture of LiAlH4 (14.58 g, 384.192 mmol, 2 equiv) in THF (450 mL) was added 365-1 (25 g, 192.096 mmol, 1 equiv) in THF (150 mL) dropwise at 0° C. under nitrogen atmosphere. The resulting mixture was stirred for 1 h at 0° C. under nitrogen atmosphere. The reaction was quenched by the addition of NaOH (2M) (75 mL) at 0° C. The resulting mixture was stirred for 1 h at room temperature. The resulting mixture was dried over anhydrous MgSO4. After filtration, the filtrate was concentrated under reduced pressure. This resulted in 365-2 (16 g, 81.55%) as a colorless oil.

### 3. Synthesis of 365-3

To a stirred solution of 365-2 (5 g, 48.956 mmol, 1 equiv) and Et3N (5.94 g, 58,747 mmol, 1.2 equiv) in DCM (100 mL) was added MsCl (6.17 g, 53.852 mmol, 1.1 equiv) dropwise at 0° C. under nitrogen atmosphere. The resulting mixture was stirred for 2 h at 0° C. under nitrogen atmosphere. The resulting mixture was diluted with MTBE. (100 mL). The DCM was removed under reduced pressure. The resulting mixture was filtered, the filter cake was washed with MTBE (10 mL). To the above filtrate was added DMF (10 mL). The MTBE was removed under reduced pressure. This resulted in 365-3 solution in DMF (10 mL).

### 4. Synthesis of 365-4

A solution of 365-3 (16.78 g, 73,243 mmol, 1.5 equiv) in DMF (170 ML) was treated with NaH (2.93 g, 73.256 mmol, 1.50 equiv, 60%) for 30 min at 0° C. under nitrogen atmosphere followed by the addition of (1-methoxycyclopropyl)methyl methanesulfonate (8.8 g, 48.829 mmol, 1 equiv) in DMF (10 mL) dropwise at 0° C. The resulting mixture was stirred for overnight at room temperature under nitrogen atmosphere. The reaction was quenched by the addition of sat. NH4Cl (aq.) (600 mL) at room temperature. The resulting mixture was extracted with EtOAc (2×200 mL). The combined organic layers were washed with brine (100 mL), then concentrated under reduced pressure. The residue was purified by silica gel column chromatography, elated with PE/EA (25:1) to afford crude product (12 g). The crude product was purified by reverse flash chromatography with the following conditions: column, C18 silica gel; mobile phase, MeCN in water (10 mmol/L NH4HCO3), 30% to 90% gradient in 35 min; detector, UV 220 nm. This resulted in 365-4 (2.9 g, 18.96%) as a light yellow oil.

### 5. Synthesis of 365-5

To a stirred solution of 365-4 (2.9 g, 9.260 mmol, 1 equiv) in EtOH (30 ML) was added hydrazine hydrate (3.71 g, 74.080 mmol, 8 equiv). The resulting mixture was stirred for overnight at 80° C. The resulting mixture was diluted with water (120 mL). The resulting mixture was extracted with CH2Cl2 (3×100 mL). The combined organic layers were dried over anhydrous Na2SO4. After filtration, the filtrate was concentrated under reduced pressure. This resulted in 365-5 (3 g, NaN) as a light yellow solid,

### 6. Synthesis of 365-6

To a stirred solution of 365-5 (3 g, 9.579 mmol, 1 equiv) in tetrahydrofuran (50 mL) was added methyl isothiocyanate (1.05 g, 14.369 mmol, 1.5 equiv). The resulting mixture was stirred for overnight at room temperature. The resulting mixture was diluted with water (50 mL). The THF was removed under reduced pressure. The precipitated solids were collected by filtration and washed with water (2×5 mL). The resulting solid was dried under vacuum. This resulted in 365-6 (3.2 g, 86.48%) as a white solid,

### 7. Synthesis of 365-7

To a stirred mixture of 365-6 (3.2 g, 8.284 mmol, 1 equiv) in H2O (40 mL) was added NaOH (1.66 g, 41.420 mmol, 5 equiv). The resulting mixture was stirred for overnight at room temperature. The mixture was acidified to pH 4 with HCl (1M). The resulting mixture was extracted with EtOAc (2×50 mL). The combined organic layers were concentrated under reduced pressure. This resulted in 365-7 (2.8 g, 91.78%) as a yellow solid.

### 8. Synthesis of 365-8

To a stirred mixture of 365-7 (2.8 g, 7.603 mmol, 1 equiv) and NaNO2 (5.25 g, 76.030 mmol, 10 equiv) in H2O (30 mL) and EtOAc (30 mL) was added HNO3 (76.03 mL, 76,030 mmol, 10 equiv, 1M) dropwise at room temperature. The resulting mixture was stirred for overnight at room temperature. The reaction was quenched by the addition of sat. NaHCO₃ (aq.) (20 mL). The resulting mixture was extracted with EtOAc (3×50 mL). The combined organic layers were concentrated under reduced pressure. The residue was purified by silica gel column chromatography, eluted with CH2Cl2/MeOH (50:1) to afford 365-8 (2.4 g, 93.89%) as a light yellow solid.

### 9. Synthesis of 365-9

To a solution of 365-8 (2.35 g, 6.989 mmol, 1 equiv) in MeCN (70 mL) and NH3H2O (70 mL, 25%) was added Cu2O (0.50 g, 1494 mmol, 0.5 equiv) in a pressure tank. The resulting mixture was stirred for overnight at 100° C. The reaction mixture was cooled to room temperature and filtered to remove insoluble solids. The MeCN was removed under reduced pressure. The resulting mixture was extracted with CH2Cl2/MeOH=10/1 (2×50 mL). The combined organic layers were concentrated under reduced pressure. The residue was purified by silica gel column chromatography, eluted with CH2Cl2/MeOH (20:1) to afford 365-9 (1.9 g, 99.81%) as a yellow solid.

### 10. Synthesis of 365-10

To a stirred solution of 365-9 (900 mg, 3.305 mmol, 1 equiv) and 1-2 (1135.30 mg, 3.966 mmol, 1.2 equiv) in DCE (20 mL) was added STAB (1400.73 mg, 6.610 mmol, 2 equiv). The resulting mixture was stirred for overnight at room temperature. The reaction was quenched by the addition of water (30 mL). The resulting mixture was extracted with CH2Cl2 (3×40 mL). The combined organic layers were concentrated under reduced pressure. The residue was purified by silica gel column chromatography, eluted with CH2Cl2/MeOH (20:1) to afford 365-10 (1.1 g, 61.12%) as a white solid.

### 11. Synthesis of 365-11

To a stirred solution of 365-10 (1.1 g, 2.020 mmol, 1 equiv) and Pyridine (0.96 g, 12.120 mmol, 6 equiv) in DCM (30 mL) was added Triphosgene (0.24 g, 0.808 mmol, 0.4 equiv) at 0° C. under nitrogen atmosphere. The resulting mixture was stirred for 1 h at 0° C. under nitrogen atmosphere. The reaction was quenched by the addition of sat. NaHCO₃ (aq.) (30 mL). The resulting mixture was extracted with CH2Cl2/MeOH=10/1 (2×30 mL). The combined organic layers were concentrated under reduced pressure. The residue was purified by reverse flash chromatography with the following conditions: column, C18 silica gel; mobile phase, MeCN in water (10 mmol/L NH4HCO3), 20% to 80% gradient in 35 min; detector, UV 254 nm. This resulted in 365-11 (720 mg, 62.69%) as a yellow solid.

### 12. Synthesis of 365

365-11 (350 mg) was separated by Prep-CHiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 6 min; Wave Length: 220/254 nm; RT1 (min): 4.382; RT2 (min): 5.07; the first peak is product) to afford 365 (143.6 mg, 41.03%) as a yellow solid.

LC-MS: (ES, m/z): [M+H]+ 569. H-NMR: (400 MHz, DMSO-d6, ppm): 50.11-0.25 (m, 2H), 30.50 (s, 2H), δ 0.82-0.86 (m, 4H), 31.43-1.66 (m, 5H), 31.75-1.92 (m, 1H), 32.19-2.25 (m, 1H), 32.59-2.63 (m, 1H), 32.65-2.75 (m, 2H), 33.15 (s, 3H), 33.25 (s, 2H), 33.53 (s, 3H), 34.44-4.48 (t, 1H), 37.01 (s, 1H), 37.32-7.36 (m, 2H), 37.43-7.46 (t, 1H), 37.66 (s, 1H), 37.70-7.72 (d, 1H), 37.80 (s, 1H), 38.35 (s, 1H).

### Example 366. Synthesis of Compound 366

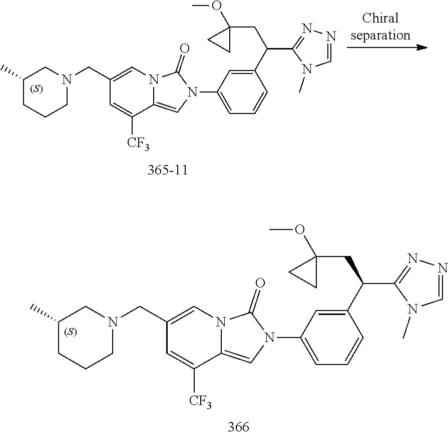

### 1. Synthesis of 366

365-11 (350 mg) was separated by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 6 min; Wave Length: 220/254 nm; RT1 (min): 4.382; RT2 (min): 5.07; the second peak was product) to afford 366 (131.5 mg, 37.57%) as a yellow solid.

LC-MS-366 (ES, m/z): [M+H]+ 569. H-NMR-366 (400 MHz, DMSO-d6, ppm): 50.11-0.25 (m, 2H), δ 0.49 (s, 2H), 60.82-0.86 (m, 4H), 61.43-1.48 (m, 5H), 61.50-1.69 (m, 4H), 61.75-1.92 (m, 1H), 62.19-2.25 (m, 1H), 62.59-2.63 (m, 1H), 52.65-2.75 (m, 2H), 63.15 (s, 3H), 63.25 (s, 2H), 53.53 (s, 3H), 64.44-4.48 (t, 1H), 67.01 (s, 1H), 67.32-7.36 (m, 2H), 67.43-7.47 (m, 1H), 67.66 (s, 1H), 67.70-7.72 (d, 1H), 67.80 (s, 1H), 58.35 (s, 1H).

### Example 367. Synthesis of Compound 367

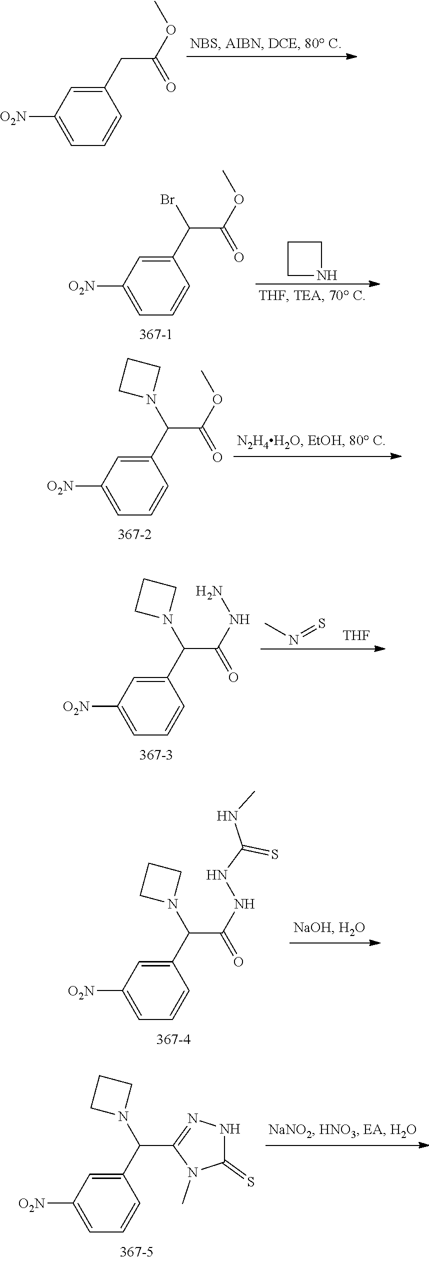

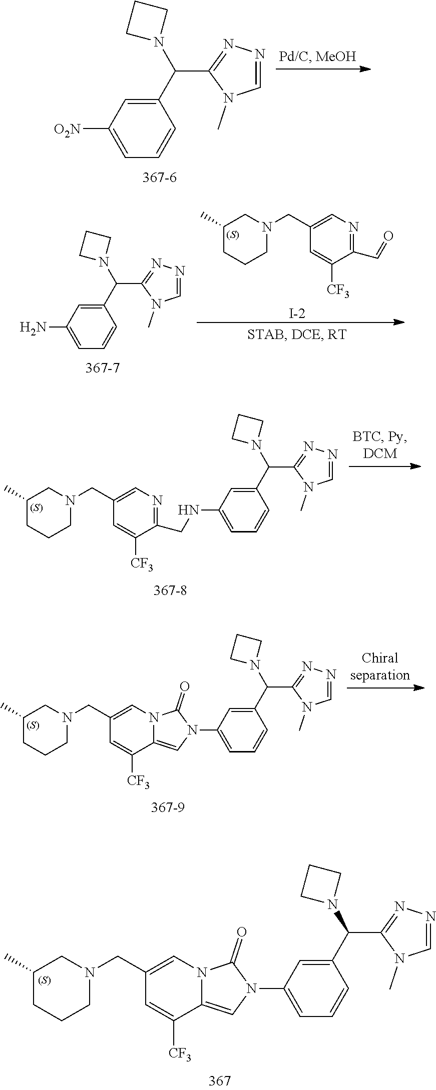

### 1. Synthesis of 367-1

Into a 500 mL 3-necked round-bottom flask were added methyl 2-(3-nitrophenyl)acetate (20 g, 102.473 mmol, 1 equiv), DCE (200 ml), NBS (27.36 g, 153.709 mmol, 1.5 equiv) and AIBN (1.68 g, 10.247 mmol, 0.1 equiv) at room temperature. The resulting mixture was stirred overnight at 80° C. under nitrogen atmosphere. The mixture was allowed to cool down to room temperature. The reaction was quenched with water (400 mL) at room temperature. The aqueous layer was extracted with CH2Cl2 (2×300 mL). The resulting mixture was concentrated under vacuum. The residue was purified by silica gel column

### 2. Synthesis of 367-2

Into a 100 mL 3-necked round-bottom flask were added 367-1 (5 g, 18,244 mmol, 1 equiv), THF (50 mL), azetidine (2.08 g, 36.488 mmol, 2 equiv) and TEA (5.54 g, 54.732 mmol, 3 equiv) at room temperature. The resulting mixture was stirred overnight at 70° C. The mixture was allowed to cool down to room temperature. The reaction was quenched with water (200 mL) at room temperature. The aqueous layer was extracted with EtOAc (2×100 mL). The resulting mixture was concentrated under vacuum. The residue was purified by silica gel column chromatography, eluted with PE/EA (6:1) to afford 367-2 (3.5 g, 70.53%) as a yellow oil.

### 3. Synthesis of 367-3

Into a 100 mL 3-necked round-bottom flask were added 367-2 (3.1 g, 12.387 mmol, 1 equiv), EtOH (30 mL) and NH2NH2·H2O (6.33 g, 123.870 mmol, 10 equiv, 98%) at room temperature. The resulting mixture was stirred overnight at 80° C. The mixture was allowed to cool down to room temperature. The reaction was quenched with water (100 mL) at room temperature. The aqueous layer was extracted with CH2Cl2/MeOH (10:1) (2×50 mL). The resulting mixture was concentrated under vacuum. The residue was purified by silica gel column chromatography, eluted with CH2Cl2/MeOH (12:1) to afford 367-3 (3 g, 96.77%) as a yellow oil.

### 4. Synthesis of 367-4

Into a 100 mL 3-necked round-bottom flask were added 367-3 (3 g, 11.988 mmol, 1 equiv), tetrahydrofuran (40 mL) and methyl isothiocyanate (1.31 g, 17.982 mmol, 1.5 equiv) at room temperature. The resulting mixture was stirred overnight at room temperature. The reaction was quenched with water (200 mL) at room temperature. The aqueous layer was extracted with EtOAc (2×100 mL). The resulting mixture was concentrated under vacuum. This resulted in 367-4 (4 g, 93.90%) as a yellow oil.

### 5. Synthesis of 367-5

Into a 100 mL 3-necked round-bottom flask were added 367-4 (4 g, 12.370 mmol, 1 equiv), 1120 (50 mL) and NaOH (1.98 g, 49.480 mmol, 4 equiv) at room temperature. The resulting mixture was stirred overnight at room temperature. The mixture was acidified to pH 5 with 1M HCl (aq.). The aqueous layer was extracted with CH2Cl2 (2×50 mL). The resulting mixture was concentrated under vacuum. The residue was purified by silica gel column chromatography, eluted with CH2Cl2/MeOH (40:1) to afford 367-5 (3.4 g, 90.01%) as a yellow solid.

### 6. Synthesis of 367-6

Into a 250 mL 3-necked round-bottom flask was added 367-5 (3.4 g, 11.134 mmol, 1 equiv), EA (34 mL), H2O (30 mL) and NaNO2 (7.68 g, 111,340 mmol, 10 equiv) at room temperature. To the above mixture was added HNO3 (110 mL, 111,340 mmol, 10 equiv, 1M) at room temperature. The resulting mixture was stirred overnight at room temperature. The mixture was basified to pH 7 with saturated NaHCO₃ (aq.). The aqueous layer was extracted with CH2Cl2 (2×100 mL). The resulting mixture was concentrated under vacuum. The residue was purified by silica gel column chromatography, eluted with CH2Cl2/MeOH (30:1) to afford 367-6 (1.9 g, 62.44%) as a yellow solid.

### 7. Synthesis of 367-7

Into a 100 mL 3-necked round-bottom flask were added 367-6 (1.9 g, 6.952 mmol, 1 equiv), MeOH (60 mL) and Pd/C (0.19 g, 10%) at room temperature. The mixture was hydrogenated at room temperature for overnight under hydrogen atmosphere using a hydrogen balloon, filtered through a Celite pad and concentrated under reduced pressure. This resulted in 367-7 (1 g, 59.12%) as a yellow oil,

### 8. Synthesis of 367-8

Into a 50 mL 3-necked round-bottom flask were added 367-7 (1 g, 4.110 mmol, 1 equiv), DCE (10 mL), 1-2 (1.18 g, 4.110 mmol, 1 equiv) and STAB (1.74 g, 8.220 mmol, 2 equiv) at room temperature. The resulting mixture was stirred for 3 h at room temperature. The reaction was quenched with NaHCO₃ (aq.) (50 mL) at room temperature. The aqueous layer was extracted with CH2Cl2/MeOH (10:1) (2×20 mL). The resulting mixture was concentrated under vacuum. The residue was purified by silica gel column chromatography, eluted with CH2Cl2/MeOH (30:1) to afford 367-8 (1 g, 47.37%) as a yellow solid.

### 9. Synthesis of 367-9

Into a 100 mL 3-necked round-bottom flask were added 367-8 (800 mg, 1,558 mmol, 1 equiv), DCM (24 mL) and Pyridine (1232.05 mg, 15.580 mmol, 10 equiv) at room temperature. To the above mixture was added Triphosgene (415.97 mg, 1.402 mmol, 0.9 equiv) at 0° C. The resulting mixture was stirred for 15 min at room temperature. The reaction was quenched with NaHCO3 (aq.) (50 mL) at room temperature. The aqueous layer was extracted with CH2Cl2/MeOH (10:1) (2×20 mL). The resulting mixture was concentrated under vacuum. The residue was purified by Prep-TLC (CH2Cl2/MeOH 12:1) to afford 367-9 (110 mg, 12.56%) as a yellow solid,

### 10. Synthesis of 367

The 367-9 (110 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 23 min; Wave Length: 220/254 nm; RT1 (min): 10.586; RT2 (min): 16.766; the second peak is product) to afford 367 (24.7 mg, 21.96%) as a yellow solid.

H-NMR-367 (400 MHz, DMSO-d6, δ ppm): 0.83-0.92 (m, 4H), 1.43-1.47 (m, 1H), 1.49-1.66 (m, 4H), 1.89-1.91 (m, 1H), 2.00-2.07 (m, 2H), 2.69-2.82 (m, 2H), 3.08-3.13 (m, 2H), 3.21-3.24 (m, 4H), 3.62 (s, 3H), 4.99 (s, 1H), 7.01 (s, 1H), 7.32-7.36 (m, 2H), 7.46-7.50 (m, 1H), 7.65-7.67 (d, 1H), 7.97 (s, 1H), 8.32 (s, 1H).

### Example 368. Synthesis of Compound 368

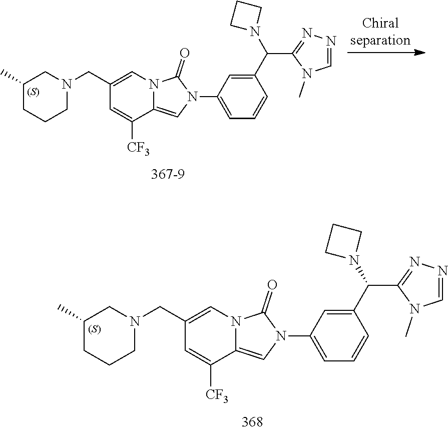

### 1. Synthesis of 368

The 367-9 (110 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 23 min; Wave Length: 220/254 nm; RT1 (min): 10.856; RT2 (min): 16.766; the first peak is product) to afford 368 (25.5 mg). H-NMR-368 δ 1.16-1.19 (m, 31H), 0.83-0.92 (m, 4H), 1.43-1.47 (m, 1H), 1.49-1.66 (m, 4H), 1.86-1.91 (m, 1H), 2.00-2.07 (ma, 2H), 2.69-2.82 (m, 2H), 3.08-3.13 (m, 2H), 3.21-3.24 (m, 4H), 3.62 (s, 3H), 4.99 (s, 1H), 7.01 (s, 1H), 7.32-7.36 (m, 2H), 7.46-7.50 (m, 1H), 7.65-7.67 (d, 1H), 7.97 (s, 1H), 8.32 (s, 1H).

### Example 369. Synthesis of Compound 369

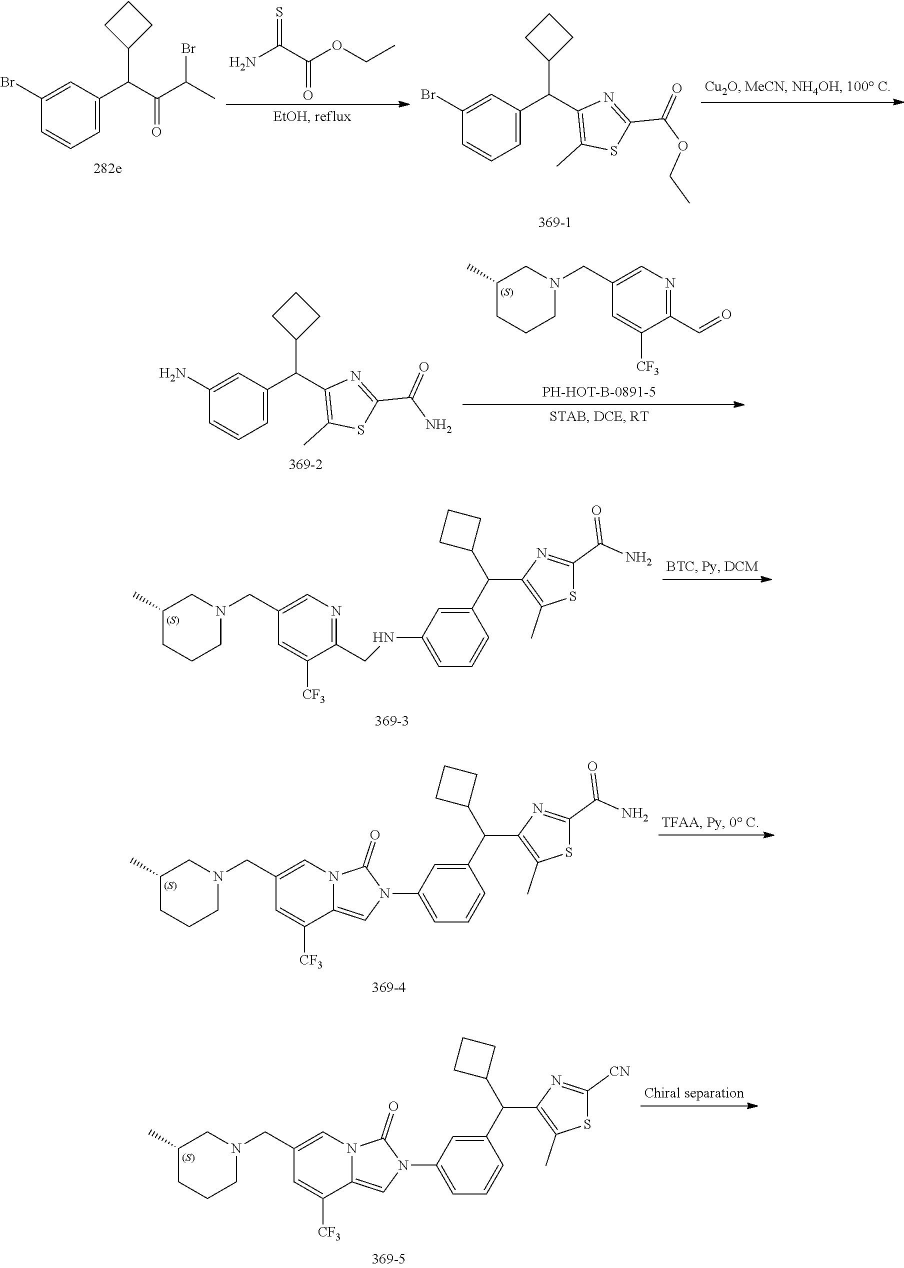

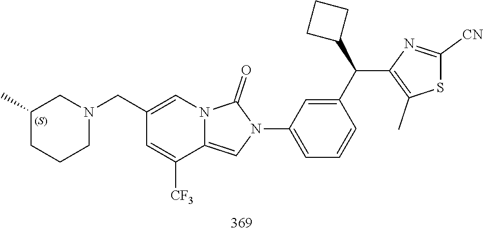

### 1. Synthesis of 369-1

To a stirred solution of 282e (7 g, 19.440 mmol, 1 equiv) in EtOH (70 mL) was added ethyl carbamothioylformate (3.88 g, 29.160 mmol, 1.5 equiv) at room temperature. The resulting mixture was stirred for 3 h at room temperature. The resulting mixture was concentrated under reduced pressure. The residue was purified by silica gel collum chromatography, eluted with PE/EA=5:1 to afford 369-1 (3 g, 35.61%) as a yellow solid.

### 2. Synthesis of 369-2

To a stirred mixture of 369-1 (2 g, 5.072 mmol, 1 equiv) in MeCN (10 mL) and NH40H (10 mL) were added Cu2O (0.15 g, 1.014 mmol, 0.2 equiv) at room temperature. The resulting mixture was stirred overnight at 100° C. The resulting mixture concentrated under reduced pressure. The residue was purified by silica gel column chromatography, eluted with PE/EA=1:1) to afford 369-2 (900 mg, 58.87%) as a yellow solid.

### 3. Synthesis of 369-3

To a stirred mixture of 369-2 (880 mg, 2.920 mmol, 1 equiv) and I-2 (835.88 mg, 2,920 mmol, 1 equiv) in DCE (9 mL) were added STAB (1856.35 mg, 8.760 mmol, 3 equiv) at room temperature. The resulting mixture was stirred overnight at room temperature. The reaction was quenched by the addition of NaHCO₃ (aq.) (30 mL) at room temperature. The aqueous layer was extracted with CH2Cl2 (2×20 mL). The resulting mixture was concentrated under reduced pressure. The residue was purified by Prep-TLC (CH2Cl2/MeOH=40:1) to afford 369-3 (900 mg, 53.92%) as a yellow solid.

### 4. Synthesis of 369-4

To a stirred solution of 369-3 (900 mg, 1.574 mmol, 1 equiv) and Pyridine (747.13 mg, 9.444 mmol, 6 equiv) in DCM (18 mL) was added Triphosgene (163.49 mg, 0.551 mmol, 0.35 equiv) at room temperature. The resulting mixture was stirred for 10 min at room temperature. The reaction was quenched by the addition of NaHCO₃ (aq.) (50 mL) at room temperature. The aqueous layer was extracted with CH2Cl2 (2×40 mL). The resulting mixture was concentrated under reduced pressure. The residue was purified by Prep-TLC (CH2Cl2/MeOH=30:1) to 369-4 (560 mg, 59.52%) as a yellow solid.

### 5. Synthesis of 369-5

To a stirred solution of 369-4 (500 mg, 0.837 mmol, 1 equiv) in pyridine (6 mL) was added TFA A (351.4 mg 1.673 mmol, 2 equiv) at room temperature. The resulting mixture was stirred for 2 h at room temperature. The reaction was quenched by the addition of NaHCO₃ (aq.) (50 mL) at room temperature. The aqueous layer was extracted with DCM (2×20 mL). The resulting mixture was concentrated under reduced pressure. The residue was purified by Prep-TLC (CH2Cl2/MeOH=15:1) to afford 369-5 (350 mg, 57.14%) as a yellow solid,

### 6. Synthesis of 369

The 369-5 (350 mg) was purified by Prep-Chiral-HPLC with the following conditions (Column: CHIRAL ART Amylose-C NEO, 2*25 cm, 5 um; Mobile Phase A: Hex (0.5% 2M N13-MeOH), Mobile Phase B: IPA; Flow rate: 20 mL/min; Gradient: 10% B to 10% B in 9 min; Wave Length: 220/254 nm; RT1 (min): 5.311; RT2 (min): 6.367; the second peak is product) to afford 369 (112.2 mg, 30.94%) as a yellow solid.

LCMS: (ES, m/z): [M+H]+ 580. H-NMR: (400 MHz, DMSO-d6, ppm, S): 0.80-0.95 (m, 4H), 1.38-1.75 (m, 6H), 1.65-1.94 (m, 6H), 2.57 (s, 3H), 2.75 (s, 2H), 3.24 (s, 3H), 4.29-4.32 (d, 1H), 7.00 (s, 1H), 7.30-7.32 (m, 1H), 7.40-7.44 (m, 1H), 7.62-7.67 (m, 2H), 7.81 (s, 1H).

### Example 370. Synthesis of Compound 370

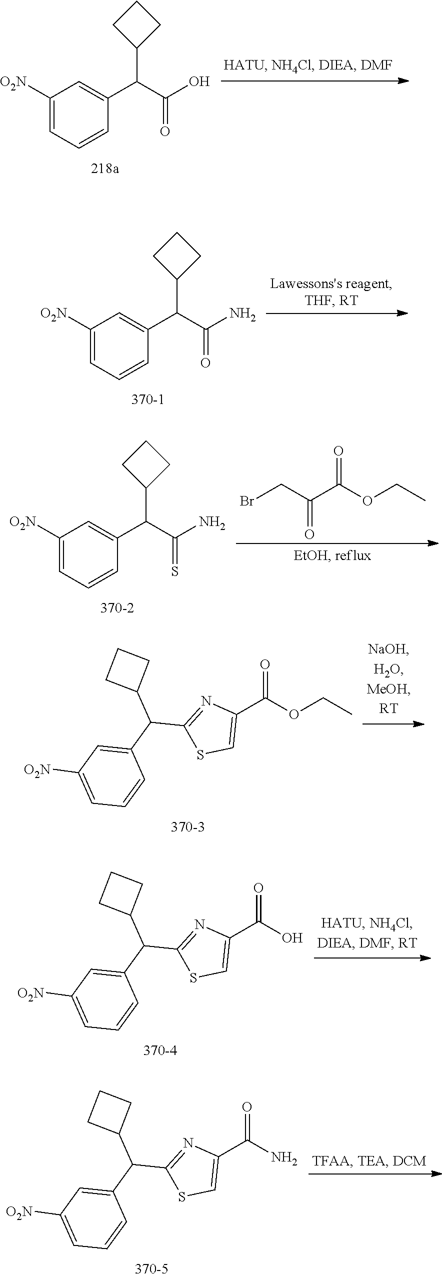

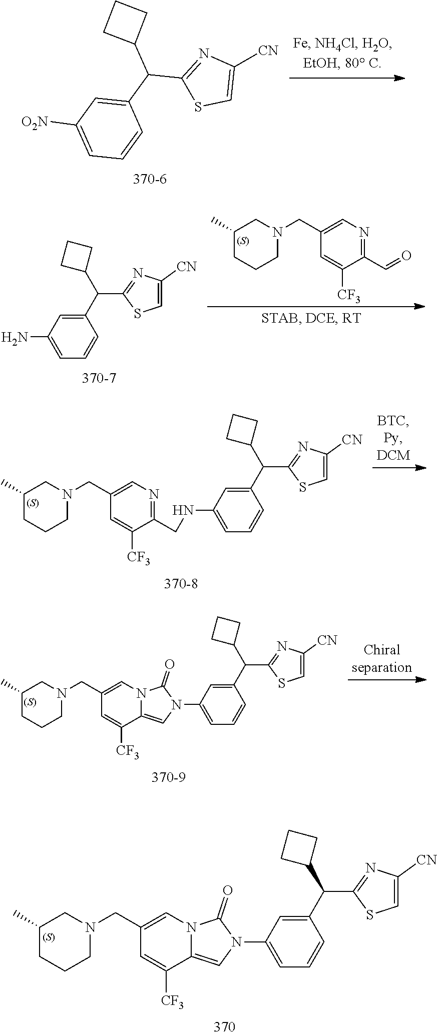

### 1. Synthesis of 370-1

To a stirred solution of 218a (90 g, 382,590 mmol, 1 equiv) and HATU (189.12 g, 497.367 mmol, 1.3 equiv) in DMF (200 mL) was added NH4Cl (40.93 g, 765.180 mmol, 2 equiv) and DIEA (148.35 g, 1147,770 mmol, 3 equiv) at room temperature. The resulting mixture was stirred for 3 h at room temperature. The resulting mixture was diluted with water (1000 mL). The aqueous layer was extracted with EtOAc (2×800 mL). The resulting mixture was concentrated under vacuum. The residue was purified by silica gel column chromatography, eluted with PE/EA (2:1) to afford 370-1 (80 g, 80.34%) as an off-white solid.

### 2. Synthesis of 370-2

To a stirred solution of 370-1 (79 g, 337.239 mmol, 1 equiv) in THF (200 mL) was added Lawesson Reagent (204.59 g, 505.858 mmol, 15 equiv) at room temperature under nitrogen atmosphere. The resulting mixture was stirred for overnight at room temperature. The reaction was quenched with water (1000 mL) at room temperature. The aqueous layer was extracted with EtOAc (2×300 mL). The resulting mixture was concentrated under vacuum. The residue was purified by silica gel column chromatography, eluted with PE/EA (10:1) to afford 370-2 (60 g, 65.39%) as an off-white solid.

### 3. Synthesis of 370-3

To a stirred solution of 370-2 (30 g, 119.847 mmol, 1 equiv) in EtOH (100 ml) was added ethyl 3-bromo-2-oxopropanoate (23.37 g, 119.847 mmol, 1 equiv) at room temperature. The resulting mixture was stirred for overnight at 80° C. The reaction was quenched with water (500 mL) at room temperature. The aqueous layer was extracted with EtOAc (2×300 mL). The resulting mixture was concentrated under vacuum. The residue was purified by silica gel column chromatography, eluted with PE/EA (10:1) to afford 370-3 (22 g, 49.28%) as a light yellow oil.

### 4. Synthesis of 370-4

To a stirred solution of 370-3 (6 g, 17.321 mmol, 1 equiv) and NaOH (2.08 g, 51.963 mmol, 3 equiv) in MeOH (60 mL) was added H2O (20 mL) at room temperature. The resulting mixture was stirred for 6 h at room temperature. The resulting mixture was diluted with water (200 mL). The mixture/residue was acidified to pH 5 with HC (aq.). The aqueous layer was extracted with EtOAc (5×100 mL). The combined organic layers were dried over anhydrous Na2SO4. After filtration, the filtrate was concentrated under reduced pressure. The crude product was used in the next step directly without further purification. This resulted in 370-4 (5 g, 77.07%) as an off-white solid.

### 5. Synthesis of 370-5

To a stirred solution of 370-4 (5 g, 15.706 mmol, 1 equiv) and HATU (7.76 g, 20.418 mmol, 1.3 equiv) in DMF (100 mL) were added DIEA (6.09 g, 47.118 mmol, 3 equiv) and NH4Cl (2.52 g, 47.118 mmol, 3 equiv) at room temperature. The resulting mixture was stirred for 3 h at room temperature. The resulting mixture was diluted with water (500 mL). The aqueous layer was extracted with EtOAc (2×200 mL). The resulting mixture was concentrated under vacuum. The residue was purified by silica gel column chromatography, eluted with PE/EA (5:1) to afford 370-5 (2.7 g, 51.46%) as an off-white solid.

### 6. Synthesis of 370-6

To a stirred solution of 370-5 (2.7 g, 8.508 mmol, 1 equiv) and TEA (2.58 g, 25.524 mmol, 3 equiv) in DCM (50 mL) was added TFA A (2.68 g, 12.762 mmol, 1.5 equiv) at 0° C. The resulting mixture was stirred for 2 h at room temperature. The resulting mixture was diluted with water (200 mL). The aqueous layer was extracted with DCM (2×100 mL). The resulting mixture was concentrated under vacuum. The crude product mixture was used in the next step directly without further purification. This resulted in 370-6 (2.4 g, 85.76%) as an off-white solid.

### 7. Synthesis of 370-7

To a stirred solution of 370-6 (2.4 g, 8.017 mmol, 1 equiv) and NH4Cl (4.29 &, 80.170 mmol, 10 equiv) in EtOH (30 mL) was added H2O (10 mL) at room temperature. The resulting mixture was stirred for 4 h at room temperature. The resulting mixture was filtered through a Celite pad, the filter cake was washed with EtOAc (2×10 mL). The filtrate was diluted with water (100 mL). The aqueous layer was extracted with EtOAc (3×50 mL). The resulting mixture was concentrated under vacuum. The residue was purified by Prep-TLC (PE/EA 2:1) to afford 370-7 (1.7 g, 74.78%) as an off-white solid.

### 8. Synthesis of 370-8

To a stirred solution of 370-7 (700 mg, 2.599 mmol, 1 equiv) and 1-2 (892.79 mg, 3,119 mmol, 1.2 equiv) in DCE (5 mL) was added STAB (1101.52 ag, 5,198 mmol, 2 equiv) at room temperature. The resulting mixture was stirred overnight at room temperature. The reaction was quenched with saturated NaHCO₃ (aq.) (100 mL) at room temperature. The aqueous layer was extracted with DCM (2×50 mL). The resulting mixture was concentrated under vacuum. The residue was purified by Prep-TLC (DCM/MeOH 30:1) to afford 370-8 (1 g, 67.74%) as a light yellow oil.

### 9. Synthesis of 370-9

To a stirred solution of 370-8 (1 g, 1853 mmol, 1 equiv) and Pyridine (1.47 g, 18,530 mmol, 10 equiv) in DCM (20 mL) was added Triphosgene (0.22 g, 0.741 mmol, 0.4 equiv) at room temperature. The resulting mixture was stirred for 10 min at room temperature. The reaction was quenched with saturated NaHCO₃ (aq.) (100 mL) at room temperature. The aqueous layer was extracted with DCM (2×50 mL). The resulting mixture was concentrated under vacuum. The residue was purified by Prep-TLC (DCM/MeOH 20:1) to afford 370-9 (700 mg, 64.78%) as a yellow solid.

### 10. Synthesis of 370

The 370-9 (380 mg) was purified by Prep-Chiral-HPLC with the following conditions (Column: CHIRALPAK IF, 2*25 cm, 5 μm; Mobile Phase A: Hex (0.5% 2M NH13-MeOH), Mobile Phase B: EtOH; Flow rate: 20 mL/min; Gradient: 20% B to 20% B in 13 min; Wave Length: 220/254 nm; RT1 (min): 8.145; RT2 (min): 10.424; the second peak is product) to afford 370 (149.4 mg, 39.12%) as a yellow solid.

LC-MS-370 (ES, m/z): [M+H]+ 566. H-NMR-370 (400 MHz, DMSO-d6, δ ppm): 0.82-0.93 (m, 4H), 1.39-1.69 (m, 5H), 1.70-1.93 (m, 6H), 2.07-2.09 (m, 1H), 2.71-2.85 (m, 2H), 3.18-3.31 (m, 3H), 4.58-4.61 (d, 1H), 7.00 (s, 1H), 7.33-7.37 (m, 2H), 7.46-7.50 (m, 1H), 7.66 (s, 1H), 7.75-7.78 (d, 1H), 7.85 (s, 1H), 8.75 (s, 1H).

### Example 371. Synthesis of Compound 371

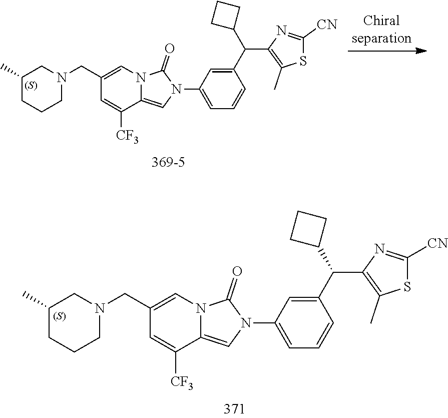

### 1. Synthesis of 371

The 369-5 (350 mg) was purified by Prep-CHIRAL-HPLC with the following conditions (Column: CHIRAL ART Amylose-C NEO, 2*25 cm, 5 μm; Mobile Phase A: Hex (0.5% 2M NH3-MeOH), Mobile Phase B: IPA; Flow rate: 20 mL/min; Gradient: 10% B to 10% B in 9 min; Wave Length: 220/254 nm; RT1 (min): 5,311; RT2 (min): 6.367; the first peak is product) to afford 371 (1134 mg, 31.27%) as a yellow solid.

LCMS: (ES, m/z): [M+H]+ 580. H-NMR: (400 MHz, DMSO-d6, ppm, S): 0.78-0.95 (m, 4H), 1.38-1.75 (m, 6H), 1.65-1.99 (m, 6H), 2.57 (s, 3H), 2.75 (s, 2H), 3.25 (s, 3H), 4.29-4.32 (d, 1H), 7.01 (s, 1H), 7.30-7.32 (m, 2H), 7.40-7.44 (m, 1H), 7.63-7.67 (m, 2H), 7.81 (s, 1H).

### Example 372. Synthesis of Compound 372

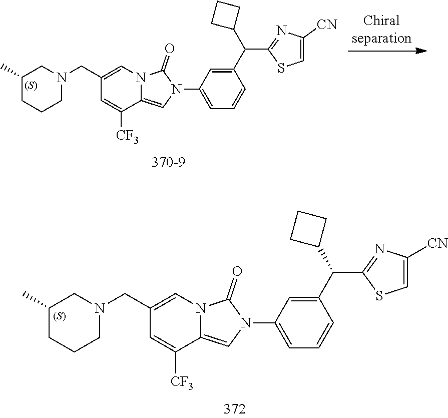

The 370-9 (380 mg) was purified by Prep-Chiral-HPLC with the following conditions (Column: CHIRALPAK IF, 2*25 cm, 5 μm; Mobile Phase A: Hex (0.5% 2M N113-MeOH), Mobile Phase B: EtOH; Flow rate: 20 mL/min; Gradient: 20% B to 20% B in 13 min; Wave Length: 220/254 nm; RT1 (min): 8.145; RT2 (min): 10.424; the first peak is product) to afford 372 (153.4 mg, 39.04%) as a yellow solid.

LC-MS-372 (ES, m/z): [M+H]+ 566. H-NMR-372 (400 MHz, DMSO-d6, δ ppm): 0.82-0.93 (m, 4H), 1.39-1.69 (m, 5H), 1.70-1.93 (m, 6H), 2.07-2.09 (m, 1H), 2.74-2.83 (m, 2H), 3.19-3.31 (m, 3H), 4.58-4.61 (d, 1H), 7.00 (s, 1H), 7.33-7.37 (m, 2H), 7.46-7.50 (m, 1H), 7.66 (s, 1H), 7.75-7.78 (d, 1H), 7.85 (s, 1H), 8.75 (s, 1H).

### Example 373. Synthesis of Compound 373

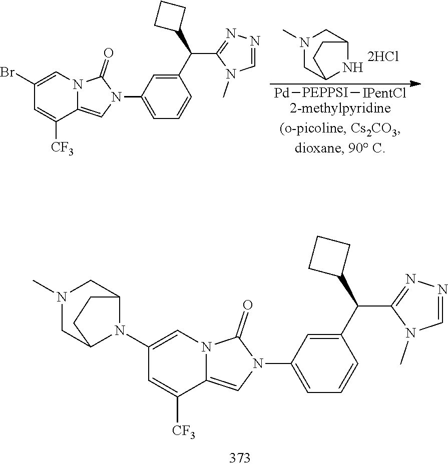

### 1. Synthesis of 373

To a stirred solution of 3-methyl-3,8-diazabicyclo[3.2.1]octane dihydrochloride (471.92 mg, 2.372 mmol, 4 equiv) and 247 C (300 mg, 0.593 mmol, 1.00 equiv) in dioxane (5 mL) were added Cs2CO3 (1930.49 mg, 5.930 mmol, 10 equiv) and Pd-PEPPSI-IPentCl 2-methylpyridine (o-picoline (249.19 mg, 0.296 mmol, 0.5 equiv) at room temperature. The resulting mixture was stirred for overnight at 90° C. under nitrogen atmosphere. The reaction was quenched by the addition of sat. NH4Cl (aq.) (10 mL) at room temperature. The resulting mixture was extracted with CH2Cl2 (3×20 mL). The resulting mixture was concentrated under reduced pressure. The residue was purified by Prep-TLC (CH2Cl2/MeOH 10:1) to afford crude product (102 mg). The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 28% B to 58% B in 8 min; Wave Length: 220 nm; RT1 (min): 7.20) to afford 373 (87.1 mg, 28.63%) as a yellow solid.

LC-MS-373 (ES, m/z): [M+H]+: 552

H-NMR-373 (400 MHz, DMSO-d6, ppm): δ 1.79-1.86 (m, 9H), δ 2.08-2.12 (m, 4H), δ 2.27-2.30 (m, 2H), δ 2.47-2.50 (m, 2H), δ 3.12-3.22 (m, 1H), δ 3.31 (s, 3H), δ 4.08 (s, 2H), δ 4.24-4.26 (d, 1H), δ 6.93 (s, 1H), δ 7.16-7.18 (m, 1H), δ 7.28 (s, 1H), δ 7.41-7.45 (m, 1H), δ 7.67-7.69 (d, 1H), δ 7.73 (s, 1H), δ 8.33 (s, 1H).

### Example 374. Synthesis of Compound 374

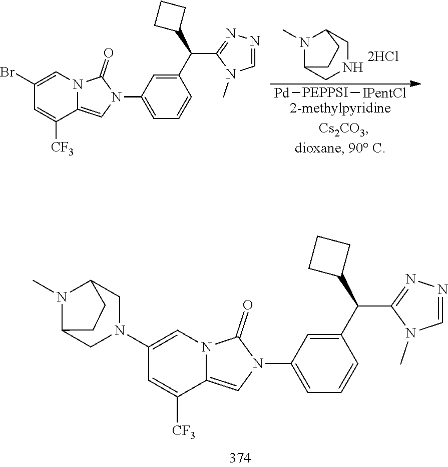

### Synthesis of 374

To a stirred solution of 8-methyl-3,8-diazabicyclo[3.2.1]octane dihydrochloride (471.92 mg, 2.372 mmol, 4 equiv) and 247 C (300 mg, 0.593 mmol, 1.00 equiv) in dioxane (5 mL) were added Cs2CO3 (1930.49 mg, 5,930 mmol, 10 equiv) and Pd-PEPPSI-IPentCl 2-methylpyridine (o-picoline) (249.19 mg, 0.296 mmol, 0.5 equiv) at room temperature. The resulting mixture was stirred for overnight at 90° C. under nitrogen atmosphere. The reaction was quenched by the addition of sat. NH4Cl (aq.) (10 mL) at room temperature. The resulting mixture was extracted with CH2Cl2 (3×20 mL). The resulting mixture was concentrated under reduced pressure. The residue was purified by Prep-TLC (CH2Cl2/MeOH 10:1) to afford crude product (90 mg). The crude product was purified by Prep-HPLC with the following conditions (Column: XBridge Prep ORD C18 Column, 30*150 mm, 5 μm; Mobile Phase A: Water (10 mmol/L NH4HCO3), Mobile Phase B: ACN; Flow rate: 60 mL/min; Gradient: 28% B to 59% B in 8 min; Wave Length: 220 nm; RT1 (min): 7.34) to afford 374 (75.3 mg, 25.85%) as a yellow solid.

LC-MS-374 (ES, m/z): [M+H]+: 552

H-NMR-374 (400 MHz, DMSO-d6, ppm): δ 1.67-1.71 (m, 3H), δ 1.72-1.82 (m, 4H), δ 2.89-1.96 (m, 2H), δ 2.01-2.12 (m, 1H), δ 2.20 (s, 3H), δ 2.67-2.72 (m, 2H), δ 3.11-3.20 (m, 5H), δ 3.45 (s, 3H), δ 4.24-4.26 (d, 1H), δ 6.75 (s, 1H), δ 7.16-7.18 (d, 1H), δ 7.24 (s, 1H), δ 7.26-7.28 (d, 1H), δ 7.69-7.71 (d, 1H), δ 7.74 (s, 1H), δ 8.32 (s, 1H).

### Example 375. Synthesis of Compound 375

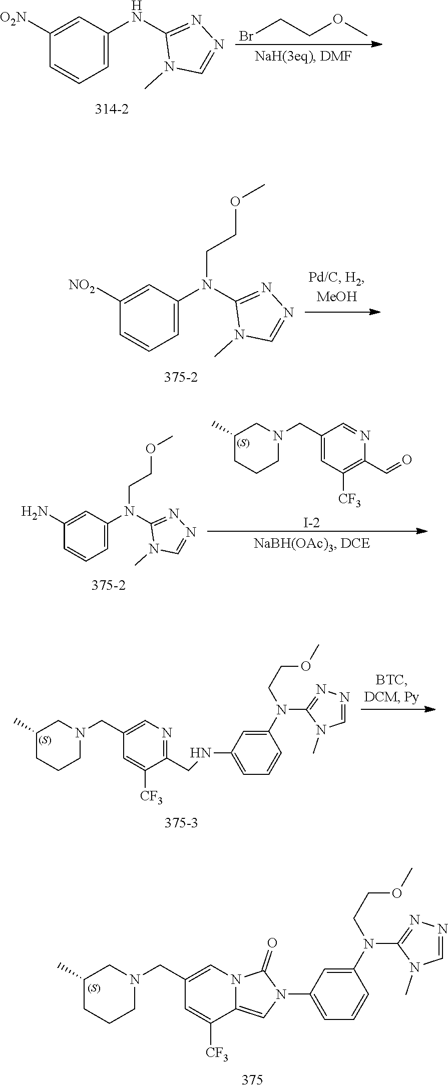

### 1. Synthesis of 375-1

To a stirred solution of 314-2 (1.5 g, 6.843 mmol, 1 equiv) and DMF (20 mL) was added NaH (0.82 g, 201529 mmol, 3 equiv, 60%) at room temperature. The mixture was stirred for 1 h at room temperature. To the above mixture was added 2-bromoethyl methyl ether (4.76 g, 34.215 mmol, 5 equiv) dropwise at room temperature. The resulting mixture was stirred for overnight at room temperature. The reaction was quenched with sat. NH4Cl (aq.) (100 mL) at room temperature. The aqueous layer was extracted with EtOAc (3×40 mL). The resulting mixture was concentrated under vacuum. The residue was purified by Prep-TLC (DCM/MeOH=40:1) to afford 375-1 (480 mg, 23.53%) as a brown oil,

### 2. Synthesis of 375-2

To a solution of 375-1 (450 mg, 1.623 mmol, 1 equiv) in 10 mL MeOH was added Pd/C (20%, 90 mg) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure. The residue was purified by Prep-TLC (DCM/MeOH=40:1) to afford 375-2 (350 mg, 81.97%) as a white solid.

### 3. Synthesis of 375-3

Into a 25 mL round-bottom flask were added 375-2 (350 mg, 1.415 mmol, 1 equiv), I-2 (486.23 ng, 1.698 mmol, 1.2 equiv), STAB (599.91 mg, 2.830 mmol. 2 equiv) and DCE (5 mL) at room temperature. The resulting mixture was stirred for overnight at room temperature. The reaction was quenched with sat. NH4Cl (aq.) (50 ml) at room temperature. The aqueous layer was extracted with EtOAc (3×20 mL). The resulting mixture was concentrated under vacuum. The residue was purified by Prep-TLC (DCM/MeOH=30:1) to afford 375-3 (300 mg, 38.50%) as a brown oil.

### 4. Synthesis of 375-0

Into a 50 mL round-bottom flask were added 375-3 (300 Ig, 0.580 mmol, 1 equiv), pyridine (366.77 mg, 4.640 mmol, 8 equiv) and DCM (10 mL) at room temperature. To the above mixture was added Triphosgene (137.60 mg, 0.464 mmol, 0.8 equiv) at 0° C. The resulting mixture was stirred for additional 5 min at 0° C. The reaction was quenched with sat. NaHCO₃ (aq.) (30 mL) at room temperature. The aqueous layer was extracted with EtOAc (3×10 mL). The resulting mixture was concentrated under vacuum. The crude product (300 mg) was purified by Prep-HPLC with the following conditions (Column: X Bridge Prep OBD C18 Column, 30*150 mm, 5 μm; Mobile Phase A: water (10 mmol/L. N14HCO3), Mobile Phase B: ACN; Flow rate: 60 mL/min; Gradient: 37% B to 67% B in 8 min, 67% B; Wave Length: 220 nm;

H-NMR: 1H NMR (400 MHz, DMSO-d6) δ0.81-0.92 (m, 4H), δ 1.41-1.46 (m, 1H), δ 1.49-1.66 (m, 4H), δ 1.85-1.90 (m, 1H), δ 2.72-2.77 (m, 2H), δ 3.22 (s, 3H), δ 3.23 (s, 2H), δ 3.27-3.34 (m, 3H), δ 3.56-3.59 (m, 2H), δ 3.95-3.98 (m, 2H), δ 6.60-6.63 (m, 1H), δ 6.99 (s, 1H), δ 7.22-7.26 (m, 2H), δ 7.31 (s, 1H), δ 7.35-7.39 (m, 2H), δ 7.65 (s, 1H), δ 8.48 (s, 1H).

### Example 376. Synthesis of Compound 376

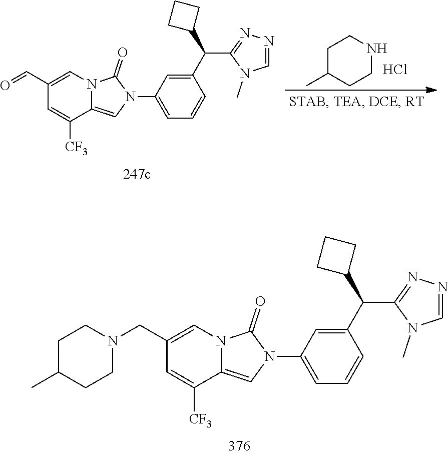

### 1. Synthesis of 376

To a stirred solution of 247c (300 mg, 0.659 mmol, 1 equiv) and piperidine, 4-methyl-hydrochloride (130.66 mag, 1.318 mmol, 2 equiv) in DCE (5 mL) were added TEA (39.56 mg, 0.659 mmol, 1 equiv) at room temperature. The mixture was stirred for 30 min at room temperature. To the above mixture was added STAB (279.21 mg, 1.318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×18 mL). The resulting mixture was concentrated under reduced pressure. The crude product (130 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Plow rate: 60 mL/min; Gradient: 43% B to 73% B in 8 min; Wave Length: 220 nm; RT1 (min): 7.60) to afford 376 (93.6 mg, 26.22%) as a yellow solid.

LC-MS: (ES, m/z): [M+H]+: 539. H-NMR: (400 MHz, DMSO-d6, ppm): δ 0.91-0.93 (d, 3H), δ 1.07-1.15 (m, 2H), δ 1.21-1.25 (m, 1H), δ 1.51-1.53 (m, 2H), δ 1.70-1.83 (m, 5H), δ 1.85-1.93 (m, 2H), δ 2.08-2.09 (d, 1H), δ 2.81-2.83 (d, 2H), δ 3.25 (s, 3H), δ 3.46 (s, 3H), δ 4.24-4.27 (m, 1H), δ 7.00 (s, 1H), δ 7.18-7.20 (d, 1H), δ 7.31 (s, 1H), δ 7.42-7.46 (m, 1H), δ 7.64-7.73 (m, 3H), δ 8.33 (s, 1H).

### Example 377. Synthesis of Compound 377

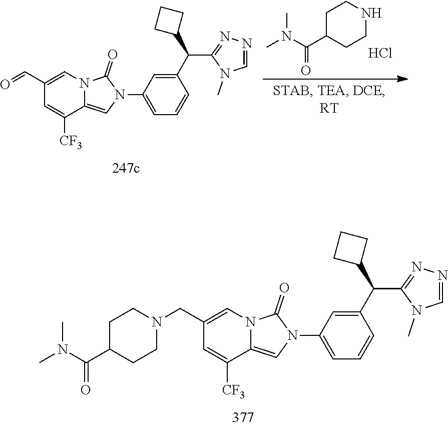

### Synthesis of 377

To a stirred solution of N,N-dimethylpiperidine-4-carboxamide hydrochloride (253.85 mg, 1.318 mmol, 2 equiv) and 247c (300 mg, 0.659 mmol, 1.00 equiv) in DCE (5 mL) were added TEA (133.31 mg, 1,318 mmol, 2 equiv) at room temperature. The mixture was stirred for 30 min at room temperature. To the above mixture was added STAB (279.21 mg, 1,318 mmol, 2 equiv) at room temperature. The resulting mixture was stirred for overnight at room temperature. The reaction was quenched by the addition of sat. NH4Cl (aq.) (10 mL) at room temperature. The resulting mixture was extracted with CH2Cl2 (3×18 mL). The resulting mixture was concentrated under reduced pressure. The crude product (68 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 22% B to 50% B in 8 min; Wave Length: 220 am; RT1 (min): 7.60) to afford 377 (47.3 mg, 18.65%) as a yellow solid.

LC-MS: (ES, m/z): [M+H]+: 596. H-NMR: (400 MHz, DMSO-d6, ppm): δ 1.51-1.62 (m, 4H), δ 1.71-1.83 (m, 5H), δ 1.92-2.15 (m, 3H), δ 2.51-2.53 (d, 1H), δ 2.80 (s, 3H), δ 2.81-2.85 (m, 2H), δ 3.00 (s, 3H), δ 3.31 (s, 1H), δ 3.33-3.35 (d, 2H), δ 3.46 (s, 3H), δ 4.24-4.27 (m, 1H), δ 7.00 (s, 1H), δ 7.18-7.20 (d, 1H), δ 7.31 (s, 1H), δ 7.42-7.46 (m, 1H), δ 7.64-7.73 (m, 3H), δ 8.33 (s, 1H).

### Example 378. Synthesis of Compound 378

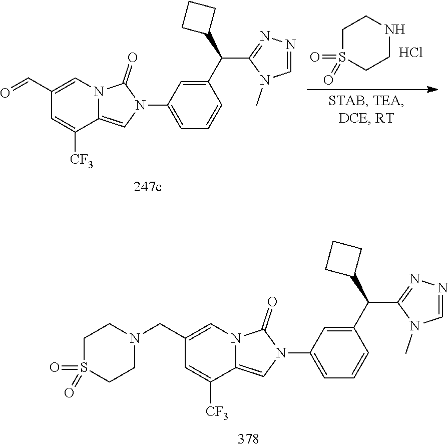

### 1. Synthesis of 378

To a stirred solution of 247c (300 mg, 0.659 mmol, 1 equiv) and thiomorpholine-1,1-dioxide hydrochloride (339.18 mg, 1.977 mmol, 3 equiv) in DCE (5 mL) was added TEA (199.97 mg, 1.977 mmol, 3 equiv) at room temperature. The resulting mixture was stirred for 1 h at room temperature. To the above mixture was added STAB (279.21 mg, 1.318 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 23% B to 53% B in 8 min, Wave Length: 220 nm; RT1 (min): 7.60) to afford 378 (66.6 mg, 17.37%) as a yellow solid.

LC-MS: (ES, m/z): [M+H]+ 575. H-NMR: (400 MHz, DMSO-d6, δ ppm): 1.67-1.87 (m, 5H), 2.08-2.10 (m, 1H), 2.95-2.96 (m, 4H), 3.11-3.12 (m, 4H), 3.20-3.23 (m, 1H), 3.43 (s, 3H), 3.50 (s, 2H), 4.25-4.27 (d, 1H), 7.05 (s, 1H), 7.19-7.21 (d, 1H), 7.32 (s, 1H), 7.42-7.46 (m, 1H), 7.68-7.72 (m, 2H), 7.78 (s, 1H), 8.33 (s, 1H).

### Example 379. Synthesis of Compound 379

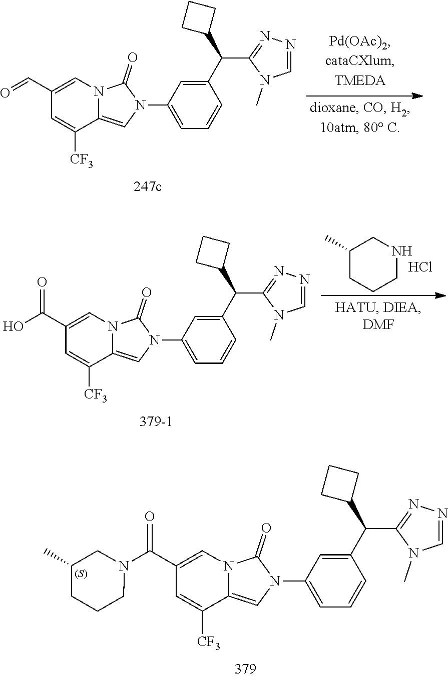

### 1. Synthesis of 379-1

Into a 1 L pressure tank reactor were added 247 h (20 g, 19.750 mmol, 1 equiv), dioxane (600 ml), TMEDA. (6.89 g, 59.250 mmol, 3 equiv), bis(adamantan-1-yl)(butyl)phosphane (1.42 g, 3.950 mmol, 0.2 equiv) and Pd(OAc)2 (0.44 g, 1.975 mmol, 0.1 equiv) at room temperature. After flushing the autoclave three times with CO/H2 (1:1), the mixture was pressurized to 10 atm with CO/H2 (1:1) at 80° C. for overnight. The mixture was allowed to cool down to room temperature. The resulting mixture was concentrated under vacuum. The residue was purified by silica gel column chromatography, eluted with CH2Cl2/MeOH (10:1) to afford 379-1 (3 g, 16.11%) as a yellow solid.

### 2. Synthesis of 379

Into a 50 mL 3-necked round-bottom flask were added DMF (20 mL), 379-1 (2 g, 4.242 mmol, 1.00 equiv), HATU (2.42 g, 6.363 mmol, 1.5 equiv), (3S)-3-methylpiperidine hydrochloride (0.86 g, 6.363 mmol, 1.5 equiv) and DIEA (1.64 g, 12.726 mmol, 3 equiv) at room temperature. The resulting mixture was stirred overnight at room temperature. The reaction was quenched with water (100 mL) at room temperature. The aqueous layer was extracted with EtOAc (40 mL). The resulting mixture was concentrated under vacuum. The crude product was purified by reverse flash chromatography with the following conditions: column, C18; mobile phase, A: water (1 mmol/L NH4HCO3), B: MeCN, 20% B to 80% B gradient in 20 min; detector, UV 254 min. This resulted in 379 (110 mg, 4.50%) as a yellow solid.

H-NMR: (400 MHz, dmso-d6, δ ppm): 0.86-0.88 (d, 3H), 1.15-1.17 (m, 1H), 1.44-1.47 (m, 1H), 1.58-1.72 (m, 3H), 1.70-1.85 (m, 5H), 2.07-2.10 (m, 1H), 2.51-2.66 (m, 1H), 2.90-3.04 (m, 1H), 3.20-3.23 (m, 1H), 3.43 (s, 3H), 3.90-4.08 (m, 2H), 4.25-4.28 (d, 1H), 7.03 (s, 1H), 7.20-7.22 (d, 1H), 7.41-7.43 (d, 1H), 7.45-7.47 (m, 1H), 7.67-7.69 (d, 1H), δ 7.75 (s, 1H), δ 7.82 (s, 1H), δ 8.33 (s, 1H).

### Example 380. Synthesis of Compound 380_P1 and 380_P2

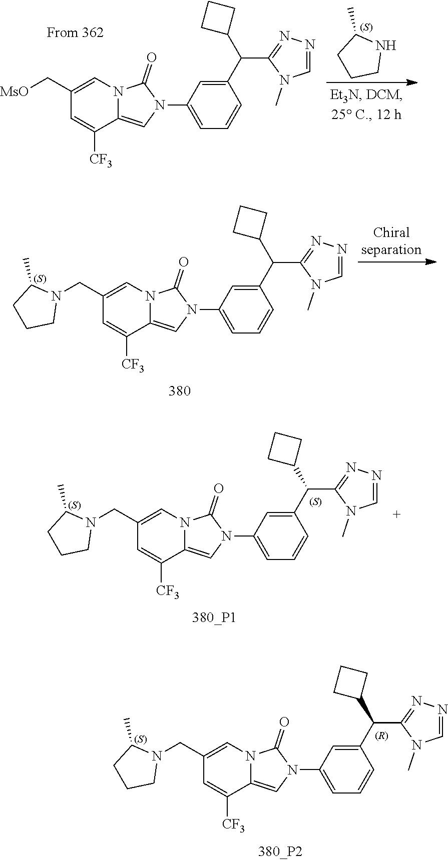

### 1. Synthesis of 380

To a solution of (2S)-2-methylpyrrolidine (64 mg, 746.91 umol, 2 equiv) and [2-[3-[cyclobutyl-(4-methyl-1,2,4-triazol-3-yl)methyl]phenyl]-3-oxo-8-(trifluoromethyl)imidazo[1,5-a]pyridin-6-yl]methyl methanesulfonate (200 mg 373.46 umol, 1 equiv) in DCM (4 mL) was added Et3N (113 mg, 1.12 mmol, 160 uL, 3 equiv) at 25° C. After addition, the mixture was stirred at this temperature for 12 h. The reaction was poured into saturated NaHCO₃ (15 mL) and the resulting mixture was extracted with DCM (2×15 mL). The organic phase was washed with brine (10 mL), dried over anhydrous Na2SO4. After filtration, the filtrate concentrated in vacuum to give a residue. The residue was purified by prep-HPLC (collum: Waters Xbridge Prep OBD C18 150*4 (0 nm*10 um; Mobile Phase A: water (NH4HCO3), Mobile Phase B: ACN; Flow rate: 50 mL/min; Gradient: 40% B to 70% B in 8 min) to afford 380 (50 mg 17%) as a yellow solid.

### 2. Synthesis of 380_P1&P2

The 380 (50 mg) was purified by Chiral separation with the following conditions (column: Phenomenex-Cellulose-2 (250 mm*30 mm, 10 um); Mobile Phase: [Neu-ETOH]; B %: 50%-50%, 5 min, Flow rate: 70 mL/min; Wave Length: 220/254 nm; RT1 (min): 6.7; RT2 (min): 9.2) to afford 380_P1 (21.8 mg, 43.1%) and 380 P2 (23.2 mg, 45.9%).

MS-380_P1: (ES, m/z): [M+H]+ 525.25.

1H-NMR-380_P1: (400 MHz, DMSO-d6) δ 8.37-8.27 (m, 1H), 7.78-7.59 (m, 3H), 7.48-7.39 (m, 1H), 7.34-7.28 (m, 1H), 7.22-7.14 (m, 1H), 7.02-6.92 (m, 1H), 4.29-4.22 (m, 1 H), 3.77-3.64 (m, 1H), 3.46-3.39 (m, 3H), 3.22-3.16 (m, 1H), 3.07-2.99 (m, 1H), 2.13 (br d, J=8.0 Hz, 1H), 2.01-1.57 (m, 9H), 1.40-1.29 (m, 1H), 1.12-1.05 (m, 3H).

LCMS-380_P2: (ES, m/z): [M+H]+ 525.25.

1H-NMR-380_P2: (400 MHz, CHLOROFORM-d) δ 8.05-8.01 (m, 1H), 7.67 (s, 2H), 7.58-7.52 (m, 1H), 7.46-7.38 (m, 1H), 7.20-7.13 (m, 1H), 6.98-6.94 (m, 1H), 6.79-6.72 (m, 1H), 4.01-3.91 (m, 1H), 3.78 (s, 1H), 3.45-3.34 (m, 4H), 3.07-2.87 (m, 2H), 2.50-2.29 (m, 2H), 2.17-2.08 (m, 1H), 2.02-1.89 (m, 3H), 1.86-1.68 (m, 5H), 1.50-1.44 (m, 1H), 1.20-1.12 (m, 3H).

### Example 381. Synthesis of Compound 381_P1 & P2

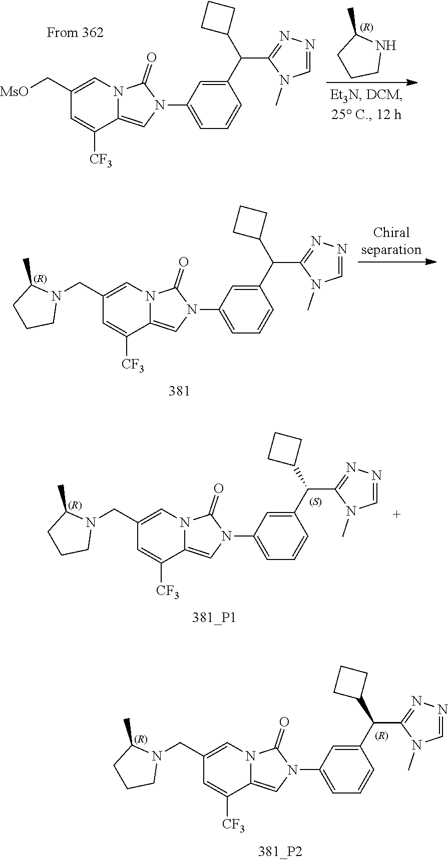

### 1. Synthesis of 381

To a solution of (2R)-2-methylpyrrolidine (90.83 ng, 746.91 umol, 2 equiv, HCl) and [2-[3-[cyclobutyl-(4-methyl-1,2,4-triazol-3-yl)methyl]phenyl]-3-oxo-8-(trifluoromethyl)imidazo[1,5-a]pyridin-6-yl]methyl methanesulfonate (200 mg, 373.46 umol, 1 equiv) in DCM (4 mL) was added Et3N (15116 mg, 1.49 mmol, 207.92 uL, 4 equiv) at 25° C. After addition, the mixture was stirred at this temperature for 12 h. The reaction was poured into saturated NaHCO₃ (15 mL) and the resulting mixture was extracted with DCM (2×15 mL). The organic phase was washed with brine (10 mL), dried over anhydrous Na2SO4. After filtration, the filtrate concentrated in vacuum to give a residue. The residue was purified by prep-HPLC (column: Waters Xbridge Prep OBD C18 150*40 mm*10 um; Mobile phase A: water (N141CO3), Mobile phase B: ACN; Flow rate: 50 mL/min; Gradient: 40% B to 70% B in 8 min; Wave Length: 220 nm) to afford 381 (40 mg, 20%) as a yellow solid.

### 2. Synthesis of 381_P1&P2

The 381 (40 mg) was purified by Chiral separation with the following conditions (column: DAICEL CHIRALPAK IC (250 mm*30 mm, 10 um); Mobile phase: [Neu-ETOH]; 13%: 65%-65%, 50 min, Flow rate: 70 mL/min; Wave Length: 220/254 nm; RT1 (min): 15.5; RT2 (min): 21.4) to afford 381 P1 (16.7 mg, 37.1%) and 381_P2 (17.7 mg, 38.9%).

MS-381_P1: (ES, m/z): [M+H]+ 525.25.

1H-NMR-381_P1: (400 MHz, DMSO-d6) δ 8.33 (s, 1H), 7.74-7.71 (m, 1H), 7.69-7.68 (m, 2H), 7.47-7.43 (m, 1H), 7.32 (s, 1H), 7.21-7.19 (m, 1H), 6.98 (s, 1H), 4.26 (d, J=10.0 Hz, 1H), 3.76-3.66 (m, 1H), 3.43 (s, 3H), 3.21 (br s, 2H), 3.04 (d, J=14.0 Hz, 1H), 2.92-2.82 (m, 1H), 2.18-2.05 (m, 2H), 2.13-1.65 (m, 8H), 1.41-1.30 (m, 1H), 1.09-1.02 (m, 3H).

MS-381_P2: (ES, m/z): [M+H]+ 525.25.

1H-NMR-381_P2: (400 MHz, DMSO-d6) δ 8.29 (s, 1H), 7.74 (s, 1H), 7.72-7.65 (m, 2H), 7.44 (d, J=8.0 Hz, 1H), 7.31 (s, 1H), 7.20 (d, J=8.0 Hz, 1H), 6.98 (s, 1H), 4.26 (d, J=12.0 Hz, 1H), 3.71 (br d, J=12.0 Hz, 1H), 3.48-3.40 (s, 3H), 3.28-3.12 (m, 2H), 3.10-2.99 (m, 1H), 2.96-2.79 (m, 1H), 2.32-2.05 (m, 2H), 2.00-1.73 (m, 1H), 1.73-1.54 (m, 7H), 1.24 (m, 1H), 1.09-1.02 (m, 3H).

### Example 382. Synthesis of Compound 382_P1 and 382_P2

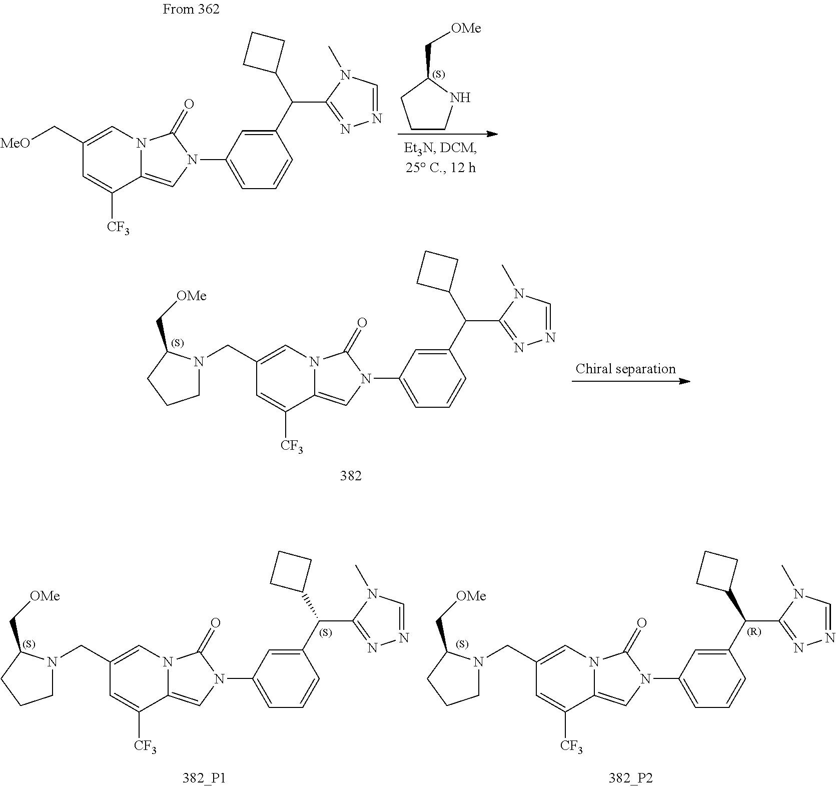

### 1. Synthesis of 382

To a solution of [2-[3-[cyclobutyl-(4-methyl-1,2,4-triazol-3-yl)methyl]phenyl]-3-oxo-8-(trifluoromethyl)imidazo[1,5-a]pyridin-6-yl]methyl methanesulfonate (450 mg, 420.14 umol, 50% purity, 1 equiv) in DCM (5 mL) was added Et3N (127.54 mg, 1.26 mmol, 3 equiv.) and (2S)-2-(methoxymethyl)pyrrolidine (96.78 mg, 840.28 umol, 103.84 uL, 2 equiv.). The mixture was stirred at 25° C. for 12 hr. The reaction was poured into water (30 mL) and the resulting mixture was extracted with DCM (2×30 mL). The organic phase was washed with brine (10 mL), dried over anhydrous Na2SO4, concentrated in vacuum to give a residue. The residue was purified by prep-HPLC(Column: Waters Xbridge Prep OBD C18 150*40 nm*10 um; Mobile phase A: [water (NH4HCO3)-ACN]; B %: ACN: Flow rate: 50 mL/min; Gradient: 40% B to 70% B in 8 min. Wave Length: 220 nm; RT1 (min): 5.1) to afford 382 (25 mg, 10% yield) as a yellow solid.

### 2. Synthesis of 382_P1&P2

382 (25 ng) was purified by Chiral separation with the following conditions (column: REGIS WHELK-O1 (250 mm*30 mm, 5 um); Mobile Phase: [Neu-IPA]; B %: 55%-55%, 18 min, Flow rate: 70 mL/min; Wave Length: 254 nm; RT1 (min): 2.38; RT2 (min): 3.00) to afford 382_P1 (5.0 mg, 20% yield) as a yellow solid 382-P1: (ES, m/z): [M+H]+ 554.2

1H-NMR-382_P1: (400 MHz, DMSO-d6) δ ppm 8.32 (s, 1H), 7.77-7.66 (m, 3H), 7.48-7.42 (m, 1H), 7.32 (s, 1H), 7.23-7.18 (m, 1H), 7.00 (s, 1H), 4.27-4.25 (d, J=8.0 Hz, 1H), 3.81-3.78 (m, 1H) 3.35 (s, 3H), 3.86-3.75 (m, 1H), 3.48-3.42 (m, 2H), 3.29-3.23 (m, 3H), 3.25-3.23 (m, 1H), 2.94-2.83 (m, 1H), 2.76-2.74 (m, 1H), 2.56-2.54 (m, 1H), 2.22-2.21 (m, 1H), 1.96-1.46 (m, 8H), 1.32-1.11 (m, 1H)

382_P2 (4.7 mg, 19% yield) as a yellow solid.

382-P2: (ES, m/z): [M+H]+ 554.2

1H-NMR-382_P2: (400 MHz, DMSO-d6) δ ppm 8.32 (s, 1H), 7.74 (s, 1H), 7.71-7.39 (m, 2H), 7.49-7.41 (m, 1H), 7.32 (s, 1H), 7.24-7.17 (m, 1H), 7.04-6.99 (m, 1H), 4.27-4.25 (m, 1H), 3.81-3.78 (m, 1H), 3.44 (s, 3H), 3.46-3.45 (m, 1H), 3.38-3.33 (m, 2H), 3.31-3.30 (m, 3H), 3.30-3.29 (m, 1H), 2.74-2.73 (m, 1H), 2.66-2.64 (m, 1H), 2.46-2.44 (m, 1H), 2.19-2.11 (m, 1H), 1.66-1.36 (m, 8H), 1.51-1.49 (m, 1H).

### Example 383. Synthesis of Compound 383 & 384

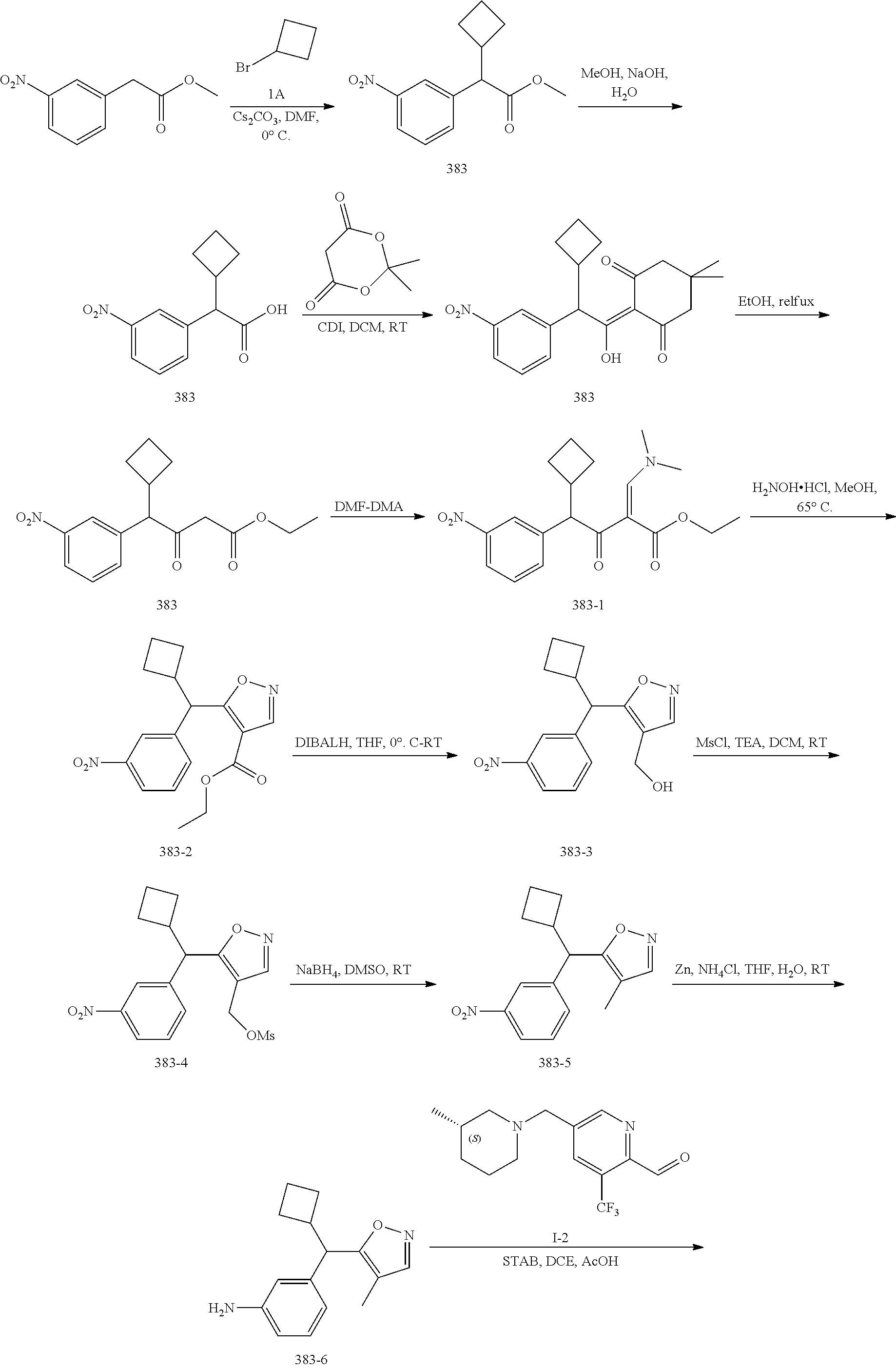

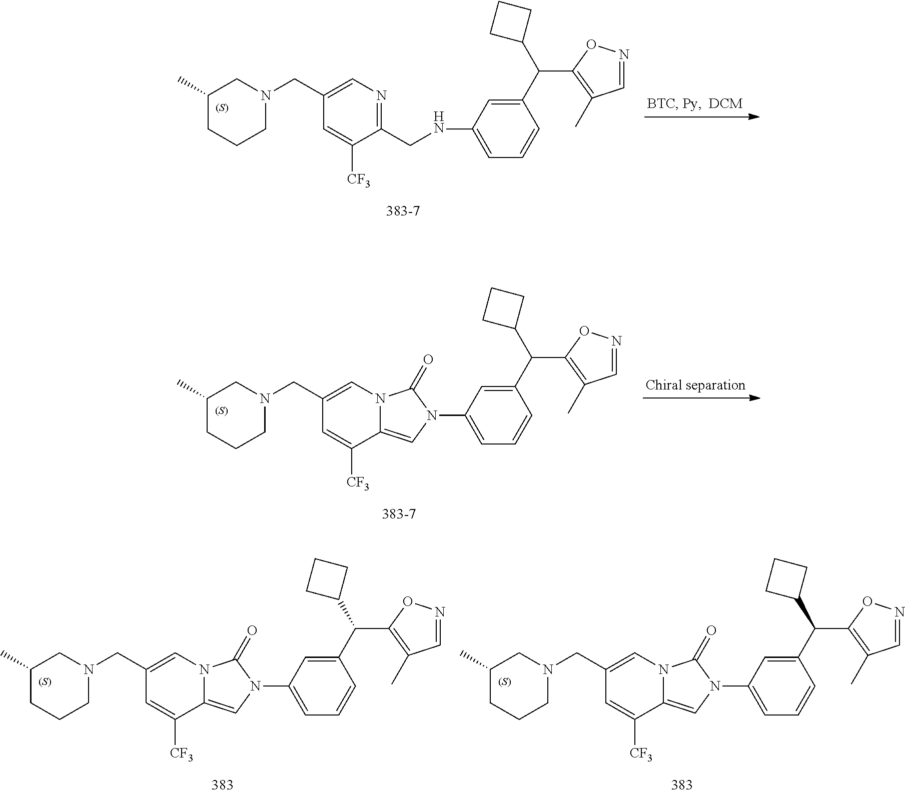

### 1. Synthesis of 383-1

Into a 2 L 3-necked round-bottom flask were added methyl 2-(3-nitrophenyl)acetate (100 g, 512.363 mmol, 1 equiv), DMF (I L) and Cs₂CO₃ (834.69 g, 2561.815 mmol, 5 equiv) at 0° C. The resulting mixture was stirred for 3 h at (PC under nitrogen atmosphere. To the above mixture was added bromocyclobutane (207.51 g, 1537,089 mmol, 3 equiv) at 0° C. The resulting mixture was stirred for additional 3 days at room temperature. The mixture acidified to pH 7 with saturated NH₄Cl (aq.) (1 L). The resulting mixture was extracted with EtOAc (3×1 L). The combined organic layers were concentrated under reduced pressure. The residue was purified by silica gel column chromatography, eluted with PE/EA (10:1) to afford 383-1 (100 g, 74.38%) as a light yellow oil.

### 2. Synthesis of 383-2

Into a 500 ml, round-bottom flask were added 383-1 (30 g, 120.353 mmol, 1 equiv), MeOH (100 mL) and THF (100 mL) at room temperature. To the above mixture was added NaOH (9.63 g, 240.706 mmol, 2 equiv) and H2O (100 mL) at room temperature. The resulting mixture was stirred for additional 3 h at 50° C. The mixture was acidified to pH 5 with 1M HCl (aq.) 400 ml. The aqueous layer was extracted with EtOAc (3×200 mL). The resulting mixture was concentrated under vacuum. This resulted in 383-2 (25 g, 83.89%) as a white solid.

### 3. Synthesis of 383-3

Into a 500 mL 3-necked round-bottom flask were added 383-2 (25 g, 106.275 mmol, 1 equiv), CDI (20.68 g, 127.530 mmol, 1.2 equiv) and DCM (300 mL) at room temperature. The mixture was stirred for 1 h at room temperature. To the above mixture was added meldrum's acid (18.38 g, 127.530 mmol, 1.2 equiv) at room temperature. The resulting mixture was stirred for overnight at room temperature. The resulting mixture was diluted with 1M HCl (500 mL). The aqueous layer was extracted with CH₂Cl₂ (3×200 mL). The resulting mixture was concentrated under vacuum. The resulting mixture was washed with 300 mL of NH₄HCO₃ (aq). The aqueous layer was extracted with CH₂Cl₂ (3×100 mL). The resulting mixture was concentrated under vacuum. The crude product 383-3 (35 g) as colorless oil was used in the next step directly without further purification.

### 4. Synthesis of 383-4

Into a 500 mL round-bottom flask were added 383-3 (35 g, 96.859 mmol, 1 equiv) and EtOH (350 mL) at room temperature. The resulting mixture was stirred for overnight at 80° C. The mixture was allowed to cool down to room temperature. The resulting mixture was diluted with water (1000 mL). The aqueous layer was extracted with EtOAc (3×400 mL). The resulting mixture was concentrated under vacuum. The residue was purified by silica gel column chromatography, eluted with PE/EA (20:1) to afford 383-4 (12 g, 38.55%) as a colorless oil.

### 5. Synthesis of 383-5

Into a 500 mL 3-necked round-bottom flask were added 383-4 (13.3 g, 43.559 mmol, 1 equiv) and DMF-DMA (150 mL) at room temperature. The resulting mixture was stirred for overnight at room temperature. The reaction was quenched by the addition of Water (300 mL) at room temperature. The aqueous layer was extracted with EtOAc (3×150 mL). The resulting mixture was concentrated under reduced pressure to afford 383-5 (15 g, 95.55%) as a Brown yellow oil. The crude product was used in the next step directly without further purification.

### 6. Synthesis of 383-6

Into a 500 mL 3-necked round-bottom flask were added 383-5 (15 g, 41.619 mmol, 1 equiv), MeOH (160 ml) and NH₂OH·HCl (4.34 g, 62.428 mmol, 1.5 equiv) at room temperature. The resulting mixture was stirred for 1 h at 65° C. under nitrogen atmosphere. The reaction was quenched by the addition of Water (100 mL) at room temperature. The aqueous layer was extracted with EtOAc (3×100 mL). The resulting mixture was concentrated under reduced pressure. The residue was purified by silica gel column chromatography, eluted with PE/EA (15:1) to afford 383-6 (8 g, 5819%) as a Brown yellow oil.

### 7. Synthesis of 383-7

Into a 250 mL 3-necked round-bottom flask were added 383-6 (4 g, 12.109 mmol, 1 equiv), THF (50 mL) and DIBALH (4.31 g, 30.273 mmol, 2.5 equiv) at 0° C. The resulting mixture was stirred for overnight at room temperature under nitrogen atmosphere. The reaction was quenched by the addition of sat. NH₄Cl (aq.) (100 mL) at room temperature. The aqueous layer was extracted with EtOAc (3×60 mL). The resulting mixture was concentrated under reduced pressure. The residue was purified by silica gel column chromatography, eluted with PE/EA (20:1) to afford 383-7 (2.3 g, 65.88%) as a light brown oil,

### 8. Synthesis of 383-8

Into a 100 mL 3-necked round-bottom flask were added 383-7 (2.3 g, 7,978 mmol, 1 equiv), DCM (30 mL), TEA (2.42 g, 23,934 mmol, 3 equiv) and MsCl (1.37 g, 11.967 mmol, 1.5 equiv) at 0° C. The resulting mixture was stirred for 2 h at room temperature. The reaction was quenched by the addition of Water (20 mL) at room temperature. The aqueous layer was extracted with CH₂Cl₂ (3×10 mL). The resulting mixture was concentrated under reduced pressure to afford 383-8 (2.5 g, 85.53%) as a light brown oil. The crude product was used in the next step directly without further purification.

### 9. Synthesis of 383-9

Into a 100 mL 3-necked round-bottom flask were added 383-8 (2.5 g, 6,823 mmol, 1 equiv), DMSO (30 mL) and NaBH4 (0.52 g, 13.646 mmol, 2 equiv) at room temperature. The resulting mixture was stirred for overnight at room temperature. The reaction was quenched by the addition of sat. NH₄Cl (aq.) (30 mL) at room temperature. The aqueous layer was extracted with EtOAc (3×30 mL). The resulting mixture was concentrated under reduced pressure. The residue was purified by silica gel column chromatography, eluted with PE/EA (5:1) to afford 383-9 (1.2 g, 64.58%) as a light yellow solid.

### 10. Synthesis of 383-10

Into a 100 mL 3-necked round-bottom flask were added 383-9 (700 mg, 2.571 mmol, 1 equiv), THF (12 mL), NH4Cl (687.52 mg, 12.855 mmol, 5 equiv) in H₂O (4 mL) and Zn (2521.04 mg, 38,565 mmol, 15 equiv) at room temperature. The resulting mixture was stirred for overnight at room temperature under nitrogen atmosphere. The resulting mixture was filtered; the filter cake was washed with THF (3×7 mL). The filtrate was concentrated under reduced pressure. The residue was purified by Prep-TLC (CH₂Cl₂/MeOH 12:1) to afford 383-10 (500 mg 80.27%) as a light yellow solid.

### 11. Synthesis of 383-11

Into a 100 ml, round-bottom flask were added 383-10 (480 mg, 1,981 mmol, 1 equiv), DCE (10 mL), I-2 (567.11 mg, 1.981 mmol, 1 equiv), AcOH (59.48 mg, 0.991 mmol, 0.5 equiv) and STAB (839.64 mg, 3.962 mmol, 2 equiv) at room temperature. The resulting mixture was stirred for 2 h at room temperature. The reaction was quenched by the addition of sat. NH₄Cl (aq.) (20 mL) at room temperature. The aqueous layer was extracted with EtOAc (3×10 mL). The resulting mixture was concentrated under reduced pressure. The residue was purified by silica gel column chromatography, eluted with CH₂Cl₂/MeOH (10:1) to afford 383-11 (500 mg, 49.24%) as a light yellow solid.

### 12. Synthesis of 383-12

Into a 100 mL round-bottom flask were added 383-11 (400 mg, 0.780 mmol, 1 equiv), DCM (10 mL), pyridine (370.33 mg, 4.680 mmol, 6 equiv) and triphosgene (92.62 mg, 0.312 mmol, 0.4 equiv) at 0° C. The resulting mixture was stirred for 10 min at 0° C. under nitrogen atmosphere. The reaction was quenched by the addition of sat. NaHCO₃ (aq.) (10 mL) at room temperature. The aqueous layer was extracted with CH₂Cl₂ (3×10 mL). The resulting mixture was concentrated under reduced pressure. The residue was purified by Prep-TLC eluted with CH₂Cl₂/MeOH (10:1) to afford 383-12 (250 mg, 59.48%) as a light yellow solid.

### 13. Synthesis of 383

The 383-12 (250 mg) was purified by Chiral separation with the following conditions (Column: CHIRALPAK IF, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 10% B to 10% B in 18 min; Wave Length: 220/254 nm; RT1 (min): 8.41; RT2 (min): 13.28; The first peak was 383. Sample Solvent: EtOH:DCM=1:1-HPLC; injection Volume: 1.2 mL; Number of Runs: 5) to afford 383 (89.7 mg, 35.88%) as a light yellow solid.

LC-MS-383 (ES, m/z): [M+H]⁺539

H-NMR-383 1H NMR (300 MHz, DMSO-d6, ppm) δ 0.82-0.86 (m, 4H), 1.58 (s, 1H), 1.60-1.63 (m, 1H), 1.66-1.76 (m, 5H), 1.78-1.82 (m, 3H), 1.84-1.97 (m, 3H), 1.99 (s, 3H), 2.73-2.77 (m, 2H), 3.15-3.18 (m, 1H), 3.25-3.32 (d, 2H), 4.34-4.37 (d, 1H), 7.01 (s, 1H), 7.29-7.34 (m, 2H), 7.37-7.48 (m, 1H), 7.66-7.77 (m, 2H), δ 7.82 (s, 1H), 8.32 (s, 1H).

LC-MS-384 (ES, m/z): [M+H]⁺ 539

H-NMR-384 1H NMR (300 MHz, DMSO-d6 ppm) δ 0.81-0.83 (m, 4H), 1.57-1.62 (m, 1H), 1.65-1.80 (m, 5H), 1.81-1.97 (m, 6H), 1.98 (s, 3H), 2.72-2.77 (m, 2H), 3.15-3.18 (m, 1H), 3.24-3.32 (d, 2H), 4.34-4.37 (d, 1H), 7.01 (s, 1H), 7.29-7.34 (m, 2H), 7.37-7.48 (m, 1H), 7.66-7.71 (m, 2H), δ 7.84 (s, 1H), 8.32 (s, 1H).

### Example 385. Synthesis of Compound 385 and 386

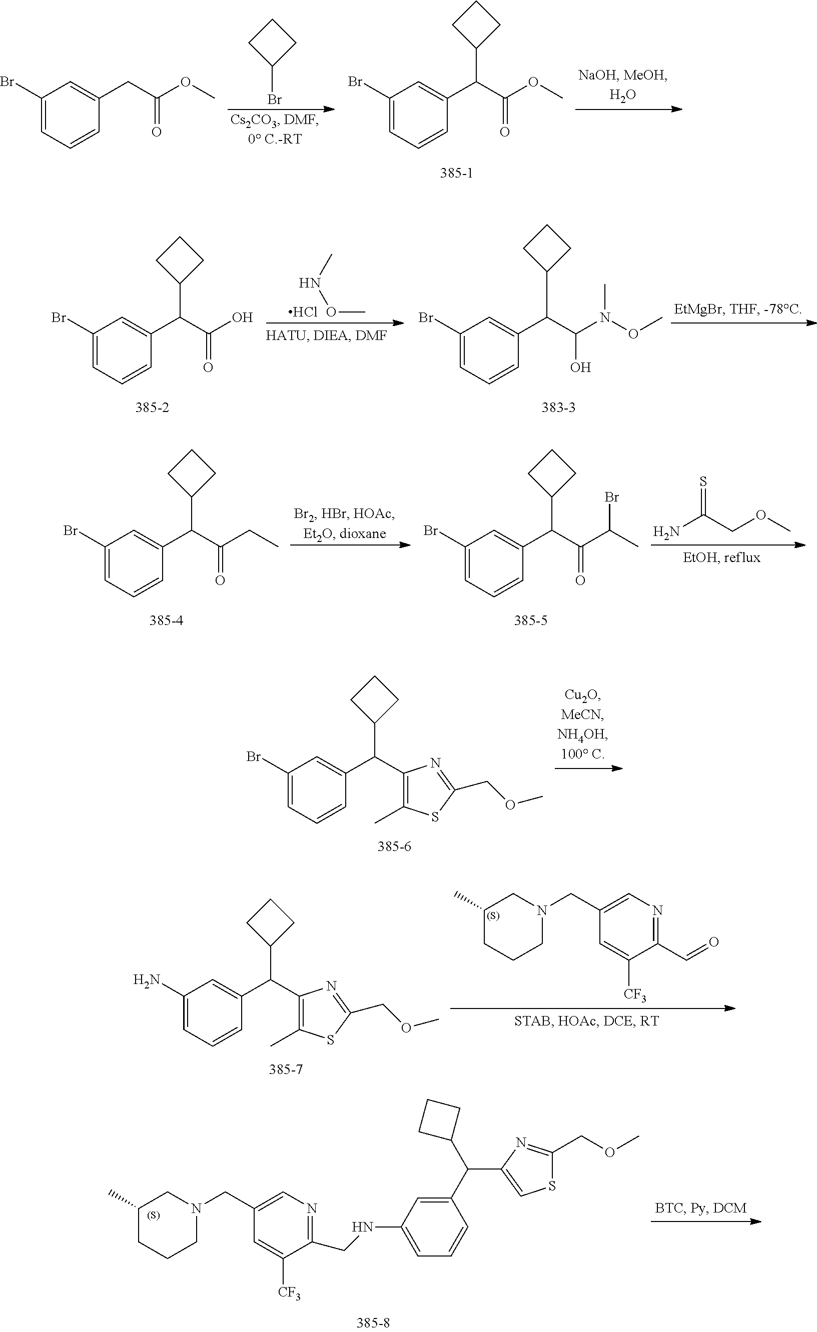

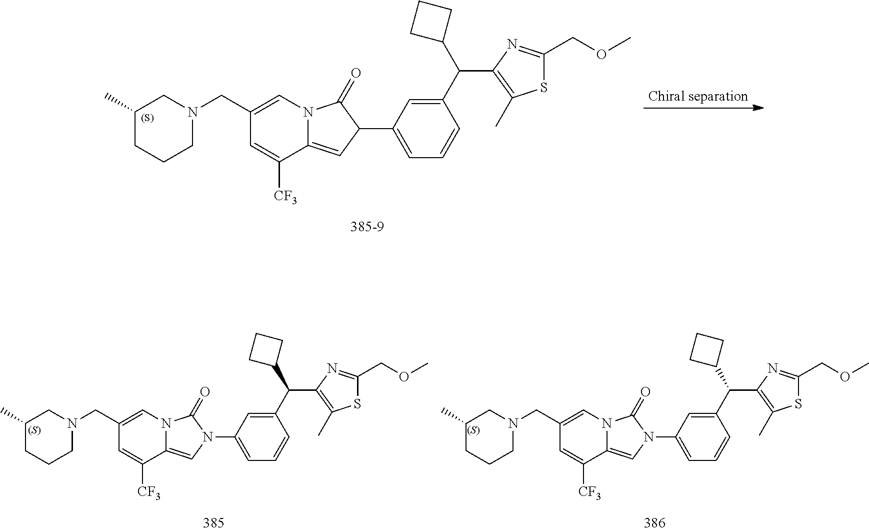

### 1. Synthesis of 385-1

To a stirred mixture of methyl 2-(3-bromophenylacetate (48 g 209.540 mmol, 1 equiv) in DMF (700 ml) was added Cs₂CO₃ (341.36 g, 1047,700 mmol, equiv) at 0° C., under nitrogen atmosphere. The resulting mixture was stirred for 3 h at 0° C.; under nitrogen atmosphere. To the above mixture was added bromocyclobutane (84.87 g& 628.620 mmol, 3 equiv) at room temperature. The resulting mixture was stirred for additional 2 days at room temperature. The resulting mixture was filtered, the filter cake was, washed with EtOAc (2×200 mL). The filtrate was quenched with saturated NH₄Cl (aq.) (2 L) at room temperature. The aqueous layer was extracted with EtOAc (2×3 L). The resulting mixture was concentrated under reduced pressure. The residue was purified by silica gel column chromatography, eluted with PE/EA=100:1 to afford 385-1. (48 g, 72.81%) as a yellow solid.

### 2. Synthesis of 385-2

To a stirred solution of 385-1 (48 g, 169.512 mmol, 1 equiv) in MeOH (600 mL)/H₂O (200 mL) was added NaOH (20.34 g, 508.536 mmol, 3 equiv) at room temperature. The resulting mixture was stirred overnight at room temperature. The mixture was neutralized to pH 6 with H₂O (aq). The aqueous layer was extracted with EtOAc (3×1000 mL) The resulting mixture was concentrated under reduced pressure. The residue was purified by silica gel column chromatography, eluted with PE/EA=1:1 to afford 385-2 (50 g, 98.64%) as a yellow solid.

### 3. Synthesis of 385-3

To a stirred solution of 385-2 (50 g, 185.778 mmol, 1 equiv) and methoxy(methyl)amine hydrochloride (36.24 g, 371.556 mmol, 2 equiv) in DMF (550 mL) was added DIEA (72.03 g, 557.334 mmol, 3 equiv) and HATU (77.70 g, 204.356 mmol, 1.1 equiv) at room temperature. The resulting mixture was stirred overnight at room temperature. The reaction was quenched with water (2 L) at room temperature. The aqueous layer was extracted with EtOAc (2×1 L). The resulting mixture was concentrated under reduced pressure. The residue was purified by silica gel column chromatography, eluted with PE/EA=5:1 to afford 385-3 (50 g, 77.58%) as a yellow oil.

### 4. Synthesis of 385-4

To a stirred solution of 385-3 (20 g, 64,060 mmol, 1 equiv) in THF (200 mL) was added EtMgBr (256 mL, 256.240 mmol, 4 equiv, 1M) dropwise at −78° C. under nitrogen atmosphere. The resulting mixture was stirred for 1 h at −78° C. under nitrogen atmosphere. The reaction was quenched with saturated NH₄Cl (aq.) (1 L) at room temperature. The aqueous layer was extracted with EtOAc (2×500 mL). The resulting mixture was concentrated under reduced pressure. The residue was purified by silica gel column chromatography, eluted with PE/EA=6:1 to afford 385-4 (18 g, 90.93%) as a yellow oil.

### 5. Synthesis of 385-5

To a stirred solution of 385-4 (15 g, 53,344 mmol, 1 equiv) in Et₂O (80 mL) and dioxane (80 mL) were added Br2 (5.97 g, 37.341 mmol, 0.7 equiv) and HBr in AcOH (40%)(3.02 g, 37.341 mmol, 0.7 equiv) at 0° C. The resulting mixture was stirred for 3 h at 0° C. The reaction was quenched with saturated NaHCO₃ (aq.) (400 mL) at room temperature. The aqueous layer was extracted with EtOAc (200 mL). The resulting mixture was concentrated under reduced pressure. The residue was purified by silica gel column chromatography, eluted with PE/EA=100:1 to afford 385-5 (12 g, 62.47%) as a colorless oil.

### 6. Synthesis of 385-6

To a stirred solution of 385-5 (5 g, 13,885 mmol, 1 equiv) in EtOH (50 mL) was added 2-methoxyethanethioamide (2.92 g, 27.770 mmol, 2 equiv) at room temperature. The resulting mixture was stirred overnight at 80° C. The reaction was quenched with water (300 mL) at room temperature. The aqueous layer was extracted with EtOAc (2×100 mL). The resulting mixture was concentrated under reduced pressure. The residue was purified by silica gel column chromatography, eluted with PE/EA=5:1 to afford 385-6 (3 g, 52.49%) as a yellow solid.

### 7. Synthesis of 385-7

To a stirred mixture of 385-6 (3.1 g, 8.463 mmol, 1 equiv) in NH40H (6 mL) and MeCN (6 mL) were added Cu₂O (0.24 g, 1.693 mmol, 0.2 equiv) at room temperature. The resulting mixture was stirred overnight at 100° C. The resulting mixture was filtered, the filter cake was washed with MeOH (10 mL). The filtrate was concentrated under reduced pressure. The residue was purified by silica gel column chromatography, eluted with CH₂Cl₂/MeOH=200:1 to afford 385-7 (1.4 g, 54.70%) as a yellow solid.

### 8. Synthesis of 385-8

To a stirred mixture of 385-7 (700 mg, 2.315 mmol, 1 equiv) and I-2 (795.17 mg, 2,778 mmol, 1.2 equiv) in DCE (7 mL) were added HOAc (138.99 mg, 2.315 mmol, 1 equiv) and STAB (1471.61 mg, 6.945 mmol, 3 equiv) at room temperature. The resulting mixture was stirred for 3 h at room temperature. The reaction was quenched with saturated NaHCO₃ (aq.) (80 mL) at room temperature. The aqueous layer was extracted with DCM (2×50 mL). The resulting mixture was concentrated under reduced pressure. The residue was purified by Prep-TLC (CH2Cl2/MeOH=30:1) to afford 385-8 (900 mg, 67.89%) as a yellow solid.

### 9. Synthesis of 385-9

To a stirred solution of 385-8 (880 mg, 1,536 mmol, 1 equiv) and pyridine (729.21 mg, 9.216 mmol, 6 equiv) in DCM (10 mL) were added Triphosgene (159.57 mg, 0.538 mmol, 0.35 equiv) at 0° C. The resulting mixture was stirred for 5 min at 0° C. The reaction was quenched with saturated NaHCO₃ (aq.) (80 mL) at room temperature. The aqueous layer was extracted with DCM (2×50 mL). The resulting mixture was concentrated under reduced pressure. The residue was purified by Prep-TLC (CH2Cl2/MeOH=20:1) to afford 385-9 (480 mg, 52.18%) as a yellow solid.

### 10. Synthesis of 385

The 385-9 (480 mg) was purified by Prep-Chiral-HPLC with the following conditions (Column: CHIRALPAK IG, 2*25 cm, 5 μm; Mobile Phase A: Hex (0.5% 2 M NH₃-MeOH), Mobile Phase B: EtOH:DCM=1:1; Flow rate: 20 mL/min; Gradient: 10% B to 10% B in 9.5 min; Wave Length: 220/254 nm; RT1 (min): 5.79; RT2 (min): 7.66; the first peak is 386 the second peak is LC-MS-385-0) to afford 386 (62.6 mg, 12.61%) as a yellow solid and 385 (188.4 mg, 37.52%) as a yellow solid.

LC-MS-385 (ES, m/z): [M+H]⁺ 599. H-NMR-385 (400 MHz, DMSO-d6, δ ppm): 0.70-0.93 (m, 4H), 1.33-1.58 (m, 1H), 1.58-1.71 (m, 5H), 1.71-1.85 (m, 6H), 2.40-2.44 (m, 3H), 2.75-2.90 (m, 2H), 3.18-3.25 (m, 3H), 3.25 (s, 3H), 4.10-4.13 (s, 1H), 4.61 (s, 2H), 7.00 (s, 1H), 7.26 (s, 1H), 7.33-7.37 (m, 1H), 7.39-7.41 (m, 1H), 7.59-7.61 (m, 1H), 7.66 (s, 1H), 7.80 (s, 1H).

LC-MS-386 (ES, m/z): [M+H]⁺ 599. H-NMR-386 (400 MHz, DMSO-d6, δ ppm): 0.70-0.93 (m, 4H), 1.33-1.60 (m, 1H), 1.60-1.71 (m, 5H), 1.71-1.92 (m, 6H), 2.44 (s, 3H), 2.73-2.77 (m, 2H), 3.18-3.25 (m, 3H), 3.30 (s, 3H), 4.10-4.13 (m, 1H), 4.58 (s, 2H), 7.00 (s, 1H), 7.25 (s, 1H), 7.33-7.41 (m, 2H), 7.59-7.61 (m, 1H), 7.66 (s, 1H), 7.80 (s, 1H).

### Example 387. Synthesis of Compound 387 and 388

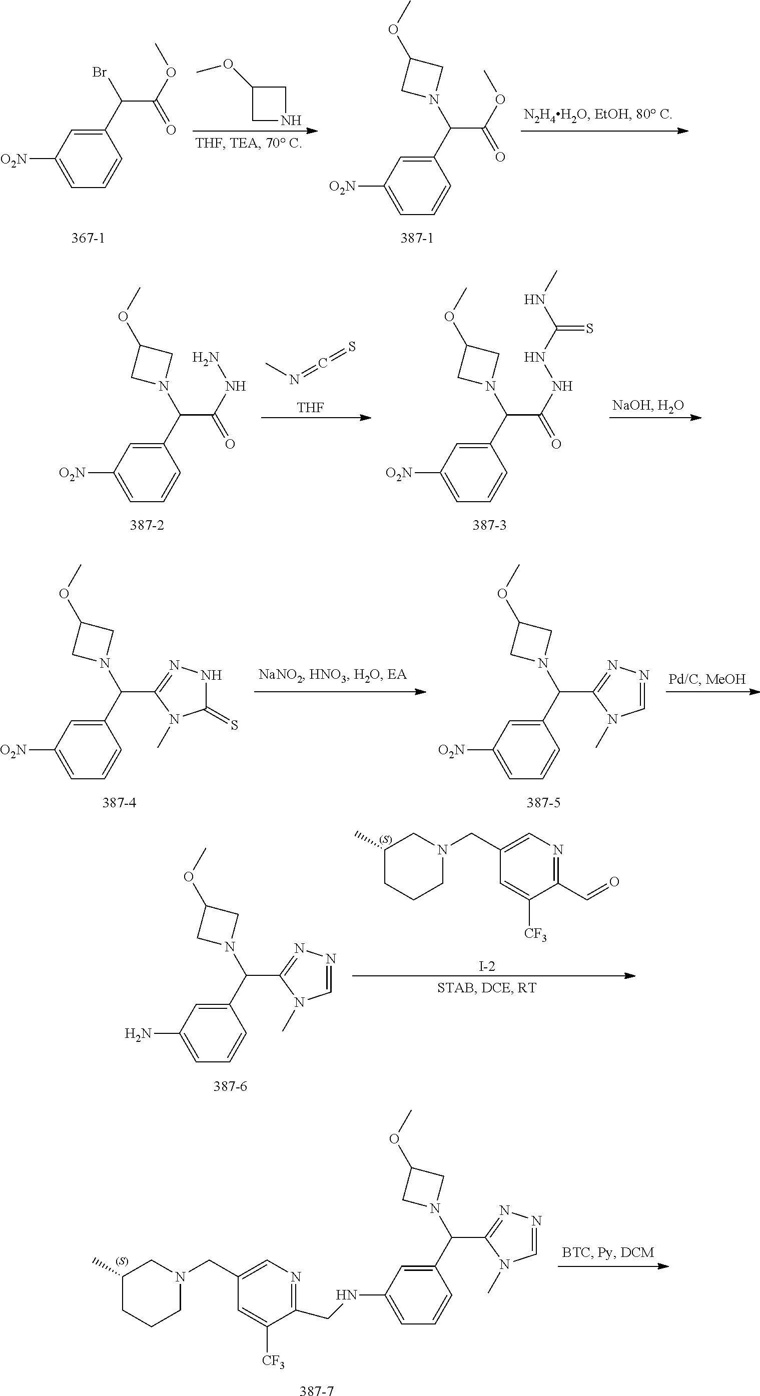

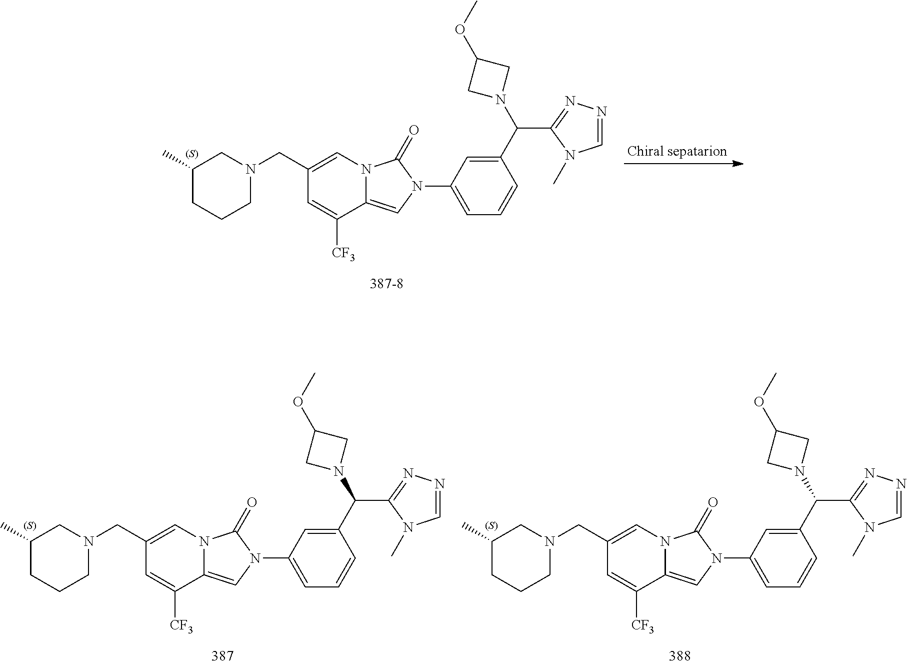

### 1. Synthesis of 387-1

A solution of 367-1 (5 g, 18.244 mmol, 1 equiv), 3-methoxyazetidine (348 g, 36.488 mmol, 2 equiv) and TEA (5.54 g, 54.732 mmol, 3 equiv) in THF (50 r L) was stirred for overnight at 70° C. under nitrogen atmosphere. The reaction was quenched by the addition of water (20 mL) at room temperature. The aqueous, layer was extracted with DCM/MeOH:10/1 (3×30 mL). The resulting mixture was concentrated under reduced pressure. The crude product 387-1 (3.36 g, 65.71%) as a yellow oil was used in the next step directly without further purification.

### 2. Synthesis of 387-2

A solution of 387-1 (3.36 g, 11.988 mmol, 1 equiv) and hydrazine hydrate (98%) (6.00 g, 119.880 mmol, 10 equiv) in EtOH (40 mL) was stirred for overnight at 80° C. The reaction was quenched with water (100 mL) at room temperature. The precipitated solids were collected by filtration. The resulting mixture was concentrated under vacuum. The crude product 387-2 (3.3 g, 98.21%) as a yellow powder was used in the next step directly without further purification.

### 3. Synthesis of 387-3

A solution of 387-2 (3.36 g, 11.988 mmol, 1 equiv) and methyl isothiocyanate (1.31 g, 17.982 mmol, 1.5 equiv) in tetrahydrofuran (40 mL) was stirred for overnight at room temperature. The reaction was quenched with water (100 mL). The aqueous layer was extracted with DCM/MeOH:10/1 (3×30 mL). The resulting mixture was concentrated under reduced pressure. The crude product 387-3 (4.2 g, 99.14%) as a yellow powder was used in the next step directly without further purification,

### 4. Synthesis of 387-4

Into a 100 mL 3-necked round-bottom flask were added 387-3 (4.8 g, 13.582 mmol, 1 equiv) and NaOH (aq, 1 mol/L) (2.17 g, 54.328 mmol, 4 equiv) stirred for overnight at room temperature. The mixture was acidified to pH 6 with HCl (1 M, 46 mL). The aqueous layer was extracted with DCM/MeOH:10/1 (3×150 ml). The resulting mixture was concentrated under reduced pressure. The crude product 387-4 (3.96 g, 86.93%) as a yellow powder was used in the next step directly without further purification.

### 5. Synthesis of 387-5

To a stirred mixture of 387-4 (3.96 g, 11,808 mmol, 1 equiv) and NaNO2 (aq. 1 mol/L, 8.15 g, 118.080 mmol, 10 equiv) in EtOAc (19.6 mL) was added HNO₃ (aq. 1 mol/L, 8.43 mL, 118.080 mmol, 10 equiv) dropwise at room temperature. The resulting mixture was stirred for overnight at room temperature. The mixture was acidified to pH 8 with saturated NaHCO₃ (aq. 10 mL). The aqueous layer was extracted with DCM (3×20 mL). The residue was purified by silica gel column chromatography, eluted with CH₂Cl₂/MeOH (20:1) to afford 387-5 (3.56 g, 99.40%) as a yellow oil.

### 6. Synthesis of 387-6

To a solution of 387-5 (3.6 g, 11,869 mmol, 1 equiv) in 120 mL MeOH was added Pd/C (10%, 0.4 g) under nitrogen atmosphere in a 500 mL, round-bottom flask. The mixture was hydrogenated at room temperature for overnight under hydrogen atmosphere using a hydrogen balloon, filtered through a filter paper pad and concentrated under reduced pressure. The crude product 387-6 (1.2 g, 36.99%) as a yellow oil was used in the next step directly without further purification.

### 7. Synthesis of 387-7

Into a 100 mL 3-necked round-bottom flask were added 387-6 (1.2 g, 4.390 mmol, 1 equiv), DCE (20 mL) and T-2 (1.26 g, 4,390 mmol, 1 equiv) at room temperature. The resulting mixture was stirred for overnight at room temperature. To the above mixture was added STAB (1.86 g, 8.780 mmol, 2 equiv) at room temperature. The resulting mixture was stirred for additional 3 h at room temperature. The reaction was quenched with sat. NaHCO₃ (aq. 20 mL) at room temperature. The aqueous layer was extracted with DCM/MeOH (10:1) (2×20 mL). The resulting mixture was concentrated under vacuum. The residue was purified by silica gel column chromatography, eluted with DCM/MeOH (30:1) to afford 387-7 (1.2 g, 50.28%) as a yellow solid.

### 8. Synthesis of 387-8

Into a 20 mL sealed tube were added 555-7 (880 mg, 1.619 mmol, 1 equiv), DCM (6.5 mL) and pyridine (1280.41 mg, 16.190 mmol, 10 equiv) at room temperature. To the above mixture was added triphosgene (432.29 mg, 1,457 mmol, 0.9 equiv) at 0° C. The resulting mixture was stirred for additional 15 min at room temperature. The reaction was quenched with sat. NaHCO₃ (aq. 10 mL) at room temperature. The aqueous layer was extracted with CH₂Cl₂/MeOH (10:1) (2×4 mL). The resulting mixture was concentrated under vacuum. The residue was purified by Prep-TLC (CH₂Cl₂/MeOH 20:1) to afford 387-8 (120 mg, 13.01%) as a yellow solid.

### 9. Synthesis of 387

The 387-8 (130 mg) was purified by Chiral separation with the following conditions (Column: Lux 5 um Cellulose-2, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 50% B to 50% B in 29 min; Wave Length: 220/254 nm; RT1 (min): 18.68; RT2 (min): 23.04; The first peak is 388, and the second peak is 387-0; Sample Solvent: EtOH-HPLC; Injection Volume: 0.6 mL; Number of Runs: 8) to afford 387-8 (120 mg, 13.01%) as a yellow solid and 387 (25.4 mg, 18.93%) as a yellow solid.

LC-MS-387 (ES, m/z): [M+H]⁺ 570. H-NMR-387 1H NMR (400 MHz, DMSO-d6 ppm) δ 0.82-0.86 (d, 4H), δ 1.44-1.47 (d, 1H), δ 1.58-1.66 (m, 4H), δ 1.87-1.92 (t, 1H), δ 2.67-2.77 (m, 2H), δ 2.90-2.94 (t, 1H), δ 2.95-2.99 (t, 1H), δ 3.15 (s, 3H), δ 3.25 (s, 2H), δ 3.41-3.45 (m, 1H), δ 3.50-3.57 (m, 1H), δ 3.59 (s, 3H), δ 4.01-4.05 (m, 1H), δ 5.02 (s, 1H), δ 7.01-7.03 (d, 1H), δ 7.32-7.36 (m, 2H), δ 7.47-7.49 (m, 1H), δ 7.67-7.69 (m, 2H), δ 7.96 (s, 1H), δ 8.33 (s, 1H).

LC-MS-388 (ES, m/z): [M+H]⁺ 570

H-NMR-388 1H NMR (400 MHz, DMSO-d6 ppm) δ 0.82-0.86 (m 4H), δ 1.44-1.47 (d, 1H), δ 1.58-1.66 (m, 4H), δ 1.87-1.92 (m, 1H), δ 2.67-2.77 (m, 2H), δ 2.90-2.94 (m, 1H), δ 2.95-2.99 (m, 1H), δ 3.15 (s, 3H), δ 3.25 (s, 2H), δ 3.41-3.45 (m, 1H), δ 3.50-3.56 (m, 1H), δ 3.59 (s, 3H), δ 4.03-4.06 (m, 1H), δ 5.02 (s, 1H), δ 7.01 (s, 1H), δ 7.32-7.36 (m, 2H), δ 7.47-7.51 (t, 1H), δ 7.67-7.70 (d, 2H), δ 7.96 (s, 1H), δ 8.33 (s, 1H).

### Example 388. Synthesis of Compound 389 and 390

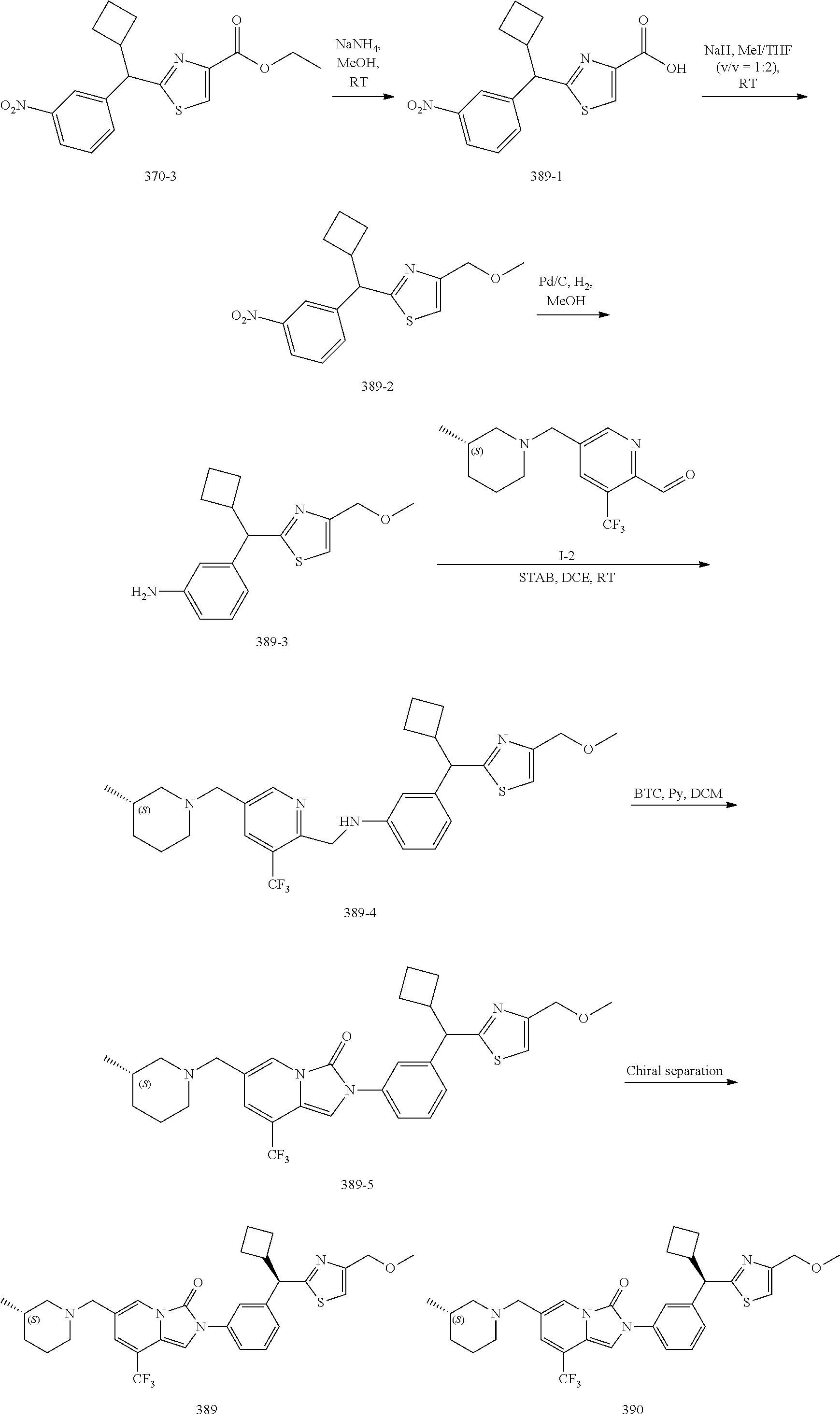

### 1. Synthesis of 389-1

To a stirred solution of 389-1 (12 g, 34,642 mmol, 1 equiv) in MeOH (150 mL) was added NaBH4 (6.55 g, 173.210 mmol, 5 equiv) at room temperature. The resulting mixture was stirred for overnight at room temperature. The reaction was quenched with saturated NH₄Cl (aq.) (500 mL) at room temperature. The aqueous layer was extracted with EtOAC (2×100 mL). The resulting mixture was concentrated under vacuum. The residue was purified by silica gel column chromatography, eluted with PE/EA (1:1) to afford 389-1 (7 g, 63.07%) as a light yellow oil.

### 2. Synthesis of 389-2

To a stirred solution of 389-1 (3 g, 9.857 mmol, 1 equiv) in THF (30 mL) was added NaI (1.97 g, 49.285 mmol, 5 equiv, 60%) at 0° C. under nitrogen atmosphere. The resulting mixture was stirred for 1 h at room temperature. To the above mixture was added MeI (15 mL) at room temperature. The resulting mixture was stirred for additional 2 h at room temperature. The reaction was quenched with saturated NH₄Cl (aq.) (100 mL) at room temperature. The aqueous layer was extracted with EtOAc (2×100 mL). The resulting mixture was concentrated under vacuum. The residue was purified by silica gel column chromatography, eluted with PE/EA (10:1) to afford 389-2 (1 g, 29.63%) as a light yellow oil.

### 3. Synthesis of 389-3

To a solution of 389-2 (1 g, 3.141 mmol, 1 equiv) in MeOH (30 mL) was added Pd/C (100 mg, 0.940 mmol, 0.30 equiv) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. The crude product was used in the next step directly without further purification. This resulted in 389-3 (620 mg, 61.60%) as a light yellow oil.

### 4. Synthesis of 389-4

To a stirred solution of 389-3 (600 mg, 2.080 mmol, 1 equiv) and 1-2 (714.73 mg, 2.496 mmol, 1.2 equiv) in DCE (8 mL) was added STAB (881.83 mg, 4.160 mmol, 2 equiv) at room temperature. The resulting mixture was stirred for 3 h at room temperature. The reaction was quenched with saturated NaHCO₃ (aq.) (50 mL) at room temperature. The aqueous layer was extracted with DCM (2×100 mL). The resulting mixture was concentrated under vacuum. The residue was purified by Prep-TLC (DCM/MeOH 40:1) to afford 389-4 (600 mg, 49.04%) as a light yellow solid.

### 5. Synthesis of 389-5

To a stirred solution of 389-4 (600 mg, 1.074 mmol, 1 equiv) and Pyridine (849.46 mg, 10.740 mmol, 10 equiv) in DCM (20 mL) was added Triphosgene (127.46 mg, 0.430 mmol, 0.4 equiv) at room temperature. The resulting mixture was stirred for 10 min at room temperature. The reaction was quenched with saturated NaHCO₃ (aq.) (100 mL) at room temperature. The aqueous layer was extracted with DCM (2×50 mL). The resulting mixture was concentrated under vacuum. The residue was purified by Prep-TLC (DCM/MeOH 20:1) to afford 389-5 (400 mg, 61.16%) as a yellow solid.

### 6. Synthesis of 389 and 390

The 389-5 (400 mg) was purified by Prep-Chiral-HPLC with the following conditions (Column: CHIRALPAK IG, 2*25 cm, 5 um; Mobile Phase A: Hex (0.5% 2M NH3-MeOH), Mobile Phase B: EtOH:DCM=1:1; Flow rate: 20 mL/min; Gradient: 10% B to 10% B in 13 min; Wave Length: 220/254 nm; RT1 (min): 8.39; RT2 (min): 10.01, the first peak is 390; the second peak is 389-0) and Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 72% B to 93% B in 8 min, Wave Length: 254 nm; RT1 (min): 7.38) to afford 390 (58.8 mg, 14.60%) as a yellow solid and 389 (63.6 ng, 15.84%) as a yellow solid.

LC-MS-389 (ES, m/z): [M+H]⁺ 585

H-NMR-389 (400 MHz, DMSO-d6, δ ppm): 0.83-0.93 (m, 4H), 1.43-1.49 (m, 1H), 1.49-1.66 (m, 4H), 1.71-1.98 (m, 6H), 2.06-2.13 (m, 1H), 2.67-2.74 (m, 2H), 3.15-3.24 (m, 1H), 3.29 (s, 2H), 3.32 (s, 3H), 4.43-4.46 (m, 3H), 7.00 (s, 1H), 7.31-7.33 (m, 2H), 7.41-7.47 (m, 2H), 7.66 (s, 1H), 7.72-7.74 (d, 1H), 7.83 (s, 1H).

LC-MS-390 (ES, m/z): [M+H]⁺ 585

H-NMR-390 (400 MHz, DMSO-d6, δ ppm): 0.83-0.93 (m, 4H), 1.43-1.53 (m, 1H), 1.58-1.98 (m, 10H), 2.01-2.12 (m, 1H), 2.67-2.77 (m, 2H), 3.15-3.24 (m, 1H), 3.29 (s, 2H), 3.32 (s, 3H), 4.43-4.46 (m, 3H), 7.00 (s, 1H), 7.31-7.33 (m, 2H), 7.41-7.47 (m, 2H), 7.66 (s, 1H), 7.72-7.74 (d, 1H), 7.83 (s, 1H).

### Example 389. Synthesis of Compound 391 and 392

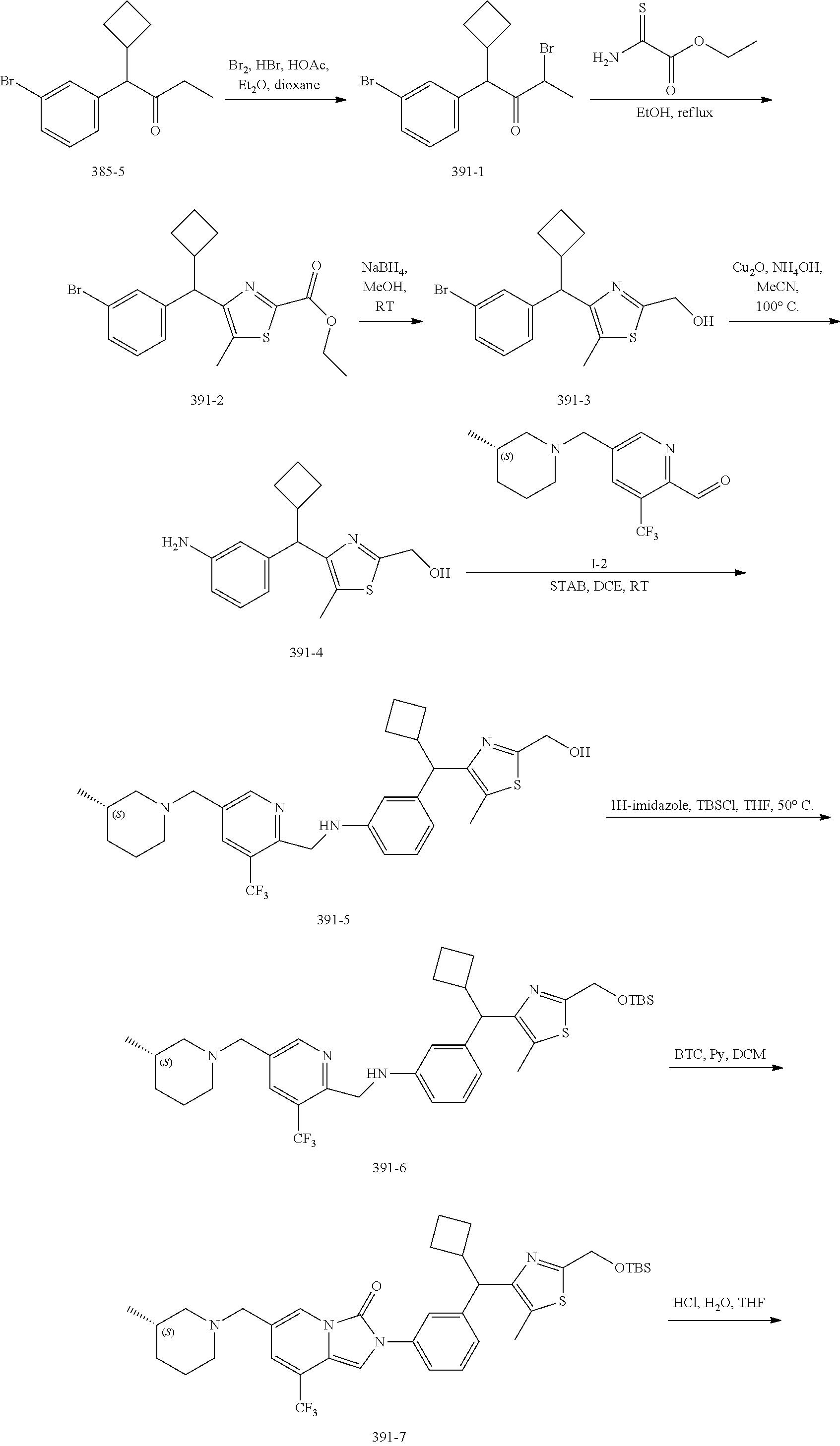

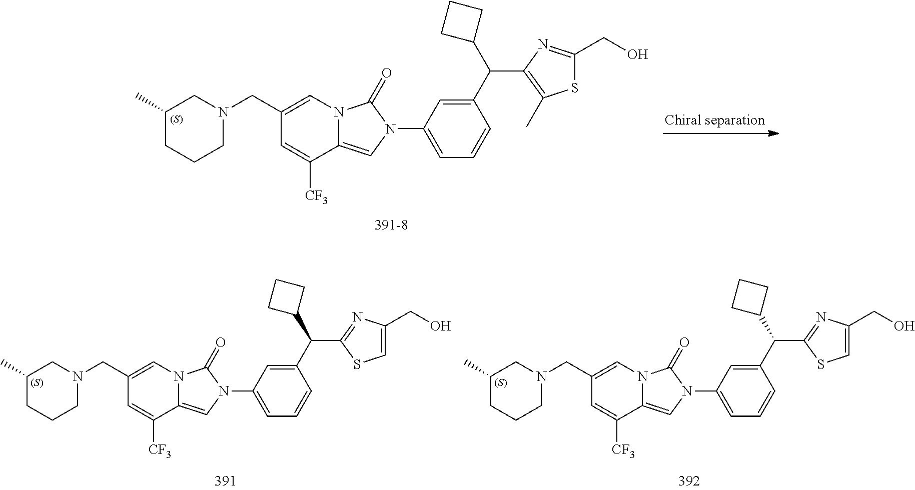

### 1. Synthesis of 391-1

To a stirred solution of 1-(3-bromophenyl)1-cyclobutylbutan-2-one (10 g, 35.563 mmol, 1 equiv) in Et₂O (100 mL) and dioxane (100 mL) were added Br₂ (3.98 g, 24.894 mmol, 0.7 equiv) and HBr in AcOH (40%)(2.01 g, 24.894 mmol, 0.7 equiv) at 0° C. The resulting mixture was stirred for 3 h at 0° C. The reaction was quenched with saturated NaHCO₃ (aq.) (400 mL) at room temperature. The aqueous layer was extracted with EtOAc (200 mL). The resulting mixture was concentrated under reduced pressure. The residue was purified by silica gel column chromatography, eluted with PE/EA=100:1 to afford 391-1 (7 g, 54.66%) as a colorless oil.

### 2. Synthesis of 391-2

To a stirred solution of 391-1 (7 g, 19.440 mmol, 1 equiv) in EtOH (70 mL) were added ethyl carbamothioylformate (5.18 g, 38.880 mmol, 2 equiv) at room temperature. The resulting mixture was stirred overnight at 80° C. The resulting mixture was diluted with water (300 mL). The aqueous layer was extracted with EtOAc (2×150 mL). The resulting mixture was concentrated under reduced pressure. The residue was purified by silica gel column chromatography, eluted with PE/EA=10:1 to afford 391-2 (3 g, 39.14%) as a yellow solid.

### 3. Synthesis of 391-3

To a stirred mixture of 391-2 (3 g, 7.608 mmol, 1 equiv) in MeOH (30 mL) was added NaBH4 (0.58 g, 15.216 mmol, 2 equiv) in portions at 0° C. The resulting mixture was stirred for 1 h at room temperature. The reaction was quenched with saturated NH4Cl (aq.) (150 mL) at room temperature. The aqueous layer was extracted with EtOAc (2×100 mL). The resulting mixture was concentrated under reduced pressure. The residue was purified by silica gel column chromatography, eluted with PE/EA=5:1 to afford 391-3 (2.5 g, 93.28%) as a yellow solid.

### 4. Synthesis of 391-4

To a stirred mixture of 391-3 (2.4 g, 6.813 mmol, 1 equiv) in MeCN (10 mL) and NH₄OH (10 mL) was added Cu2O (0.19 g, 1.363 mmol, 0.2 equiv) at room temperature. The resulting mixture was stirred overnight at 100° C. The resulting mixture was filtered, the filter cake was washed with MeOH (2×10 mL). The filtrate was concentrated under reduced pressure. The residue was purified by Prep-TLC (CH₂Cl₂/MeOH=30:1) to afford 391-4 (700 mg, 35.63%) as a yellow solid.

### 5. Synthesis of 391-5

To a stirred mixture of 391-4 (680 mg, 2.358 mmol, 1 equiv) and 1-2 (810.02 mg, 2.830 mmol, 1.2 equiv) in DCE (7 mL) was added STAB (1499.11 mg, 7.074 mmol, 3 equiv) at room temperature. The reaction was quenched with saturated NaHCO₃ (aq.) (30 mL) at room temperature. The aqueous layer was extracted with DCM (20 mL). The resulting mixture was concentrated under reduced pressure. The residue was purified by Prep-TLC (CH₂Cl₂/MeOH=20:1) to afford 391-5 (910 mg, 69.08%) as a yellow solid.

### 6. Synthesis of 391-6

To a stirred solution of 391-5 (890 mg, 1,593 mmol, 1 equiv) and 1H-imidazole (216.89 mg, 3.186 mmol, 2 equiv) in THF (10 mL) were added TBSCl (480.18 mg, 3.186 mmol, 2 equiv) at room temperature. The resulting mixture was stirred for 2 h at room temperature. The reaction was quenched with water (50 mL) at room temperature. The aqueous layer was extracted with DCM (2×20 mL). The resulting mixture was concentrated under reduced pressure. The residue was purified by Prep-TLC (CH₂Cl₂/MeOH=20:1) to afford 391-6 (1 g, 93.28%) as a yellow solid.

### 7. Synthesis of 391-7

To a stirred solution of 391-6 (1 g, 1.486 mmol, 1 equiv) and pyridine (0.71 g, 8.916 mmol, 6 equiv) in DCM (12 mL) were added Triphosgene (0.15 g, 0.520 mmol, 0.35 equiv) at 0° C. The resulting mixture was stirred for 5 min at room temperature. The reaction was quenched with saturated NaHCO₃ (aq.) (100 mL) at room temperature. The aqueous layer was extracted with CH₂Cl₂ (2×50 ml). The resulting mixture was concentrated under reduced pressure. The residue was purified by Prep-TLC (CH₂Cl₂/MeOH=15:1) to afford 391-7 (800 mg, 77.02%) as a yellow solid.

### 8. Synthesis of 391-8

To a stirred solution of 391-7 (800 mg, 1.145 mmol, 1 equiv) in THF (4 mL) was added HCl (4 mL, 2M) at room temperature. The reaction was quenched with saturated NaHCO₃ (aq.) (50 mL) at room temperature. The aqueous layer was extracted with DCM (3×30 mL). The resulting mixture was concentrated under reduced pressure. The residue was purified by Prep-TLC (CH₂Cl₂/MeOH=15:1) to afford 391-8 (250 mg, 37.36%) as a yellow solid.

### 9. Synthesis of 391

The 391-8 (260 mg) was purified by Prep-Chiral-HPLC with the following conditions (Column: CHIRALPAK IF, 2*25 cm, 5 μm; Mobile Phase A: Hex (0.5% 2 M NH₃-MeOH), Mobile Phase B: EtOH; Flow rate: 20 mL/min; Gradient: 15% B to 15% B in 8.5 min; Wave Length: 220/254 nm; RT1 (min): 4.967; RT2 (min): 7.185; the first peak is 392; the second peak is 391-0) to afford 391 (111.7 mg, 42.19%) as a yellow solid.

LC-MS-391: (ES, m/z): [M+H]⁺ 585. H-NMR-391: (400 MHz, DMSO-d6, δ ppm): 0.70-0.93 (m, 4H), 1.33-1.48 (m, 1H), 1.48-1.88 (m, 11H), 2.43 (s, 3H), 2.73-2.77 (m, 2H), 3.17-3.24 (m, 3H), 4.08-4.11 (m, 1H), 4.59-4.62 (m, 2H), 5.86-5.89 (m, 1H), 7.00 (s, 1H), 7.27 (s, 1H), 7.32-7.37 (m, 1H), 7.37-7.41 (m, 1H), 7.58-7.60 (m, 1H), 7.66 (s, 1H), 7.80 (s, 1H).

LC-MS-392: (ES, m/z): [M+H]⁺ 585. H-NMR-392: (400 MHz, DMSO-d6, δ ppm): 0.70-0.93 (m, 4H), 1.33-1.48 (m, 1H), 1.48-1.88 (m, 1H), 2.43 (s, 3H), 2.73-2.77 (m, 2H), 3.17-3.24 (m, 3H), 4.08-4.11 (m, 1H), 4.59-4.62 (m, 2H), 5.86-5.89 (m, 1H), 7.00 (s, 1H), 7.27 (s, 1H), 7.32-7.37 (m, 1H), 7.37-7.41 (m, 1H), 7.58-7.60 (m, 1H), 7.66 (s, 1H), 7.80 (s, 1H).

### Example 390. Synthesis of Compound 393 and 394

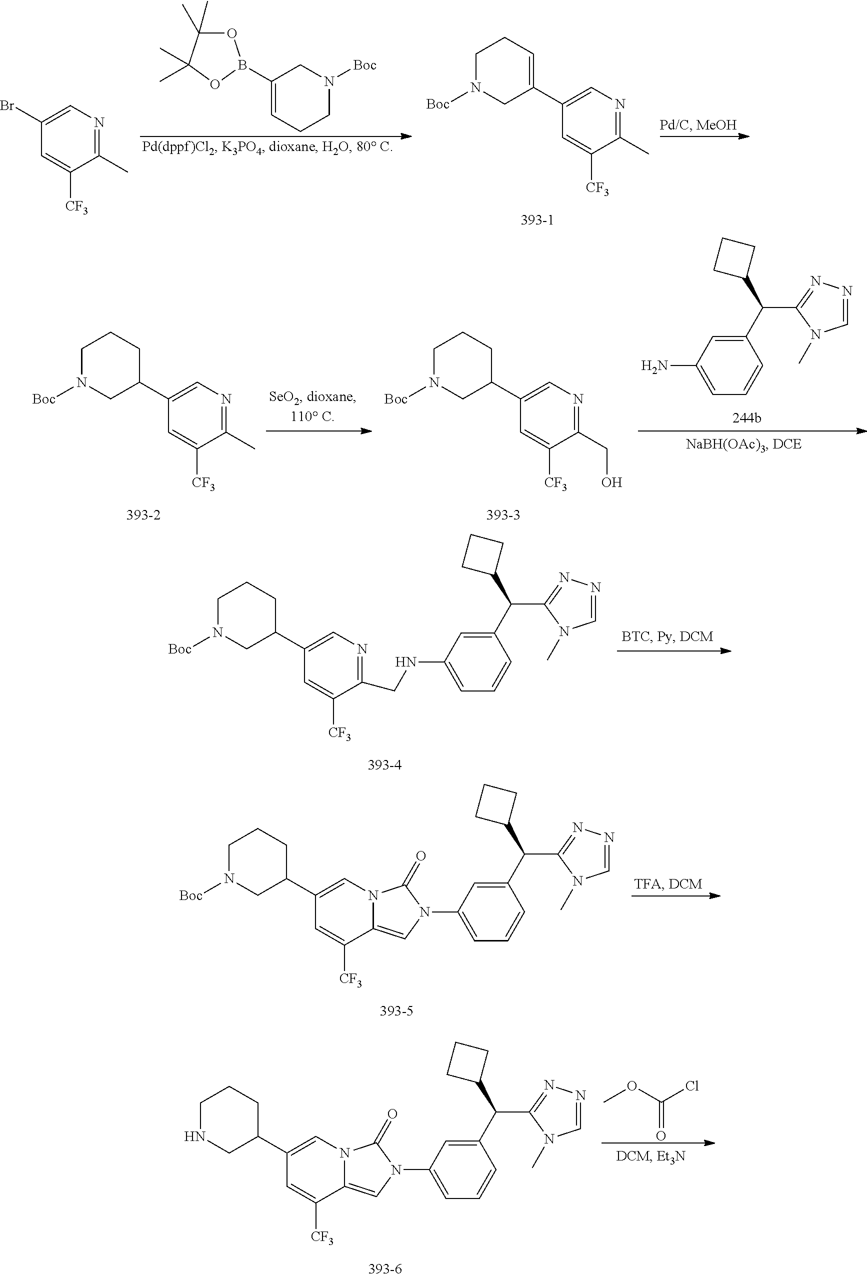

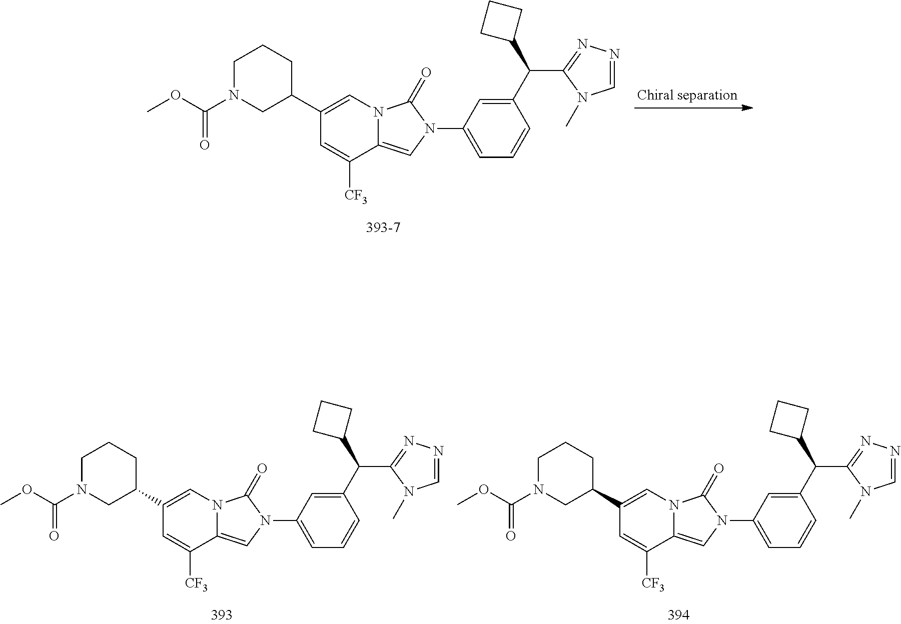

### 1. Synthesis of 393-1

A solution of 5-bromo-2-methyl-3-(trifluoromethyl)pyridine (3.88 g, 16,170 mmol, 1 equiv) and tert-butyl 3-(4,4,5,5-tetramethyl-1,3,2-dioxaborolan-2-yl)-5,6-dihydro-2H-pyridine-1-carboxylate (5 g, 16.170 mmol, 1.00 equiv) in dioxane (40 mL) and H2O (10 mL) was treated with K3PO4 (10.30 g, 48.510 mmol, 3 equiv), and Pd(dppf)Cl2 (1.77 g, 2.425 mmol, 0.15 equiv) at room temperature. The resulting mixture was stirred for overnight at 80° C. under nitrogen atmosphere. The reaction was quenched by the addition of water (100 mL) at room temperature. The resulting mixture was extracted with DCM (3×100 mL). The combined organic layers were concentrated under reduced pressure. The residue was purified by silica gel column chromatography, eluted with PE/EA (20:1) to afford 393-1 (5.03 g, 85.41%) as a light yellow solid.

### 2. Synthesis of 393-2

To a solution of 393-1 (5 g, 14.604 mmol, 1 equiv) in MeOH (150 mL) was added Pd/C (10%, 1.5 g) under nitrogen atmosphere in a 1 L round-bottom flask. The mixture was hydrogenated at room temperature for overnight under hydrogen atmosphere by using a hydrogen balloon. The resulting mixture was filtered, the filter cake was washed with MeOH (100 mL). The filtrate was concentrated under reduced pressure to afford 393-2 (3.6 g, 68.00%) as a light yellow solid.

### 3. Synthesis of 393-3

A solution of 393-2 (3.6 g, 10.454 mmol, 1 equiv) in dioxane (36 mL) was treated with SeO₂ (3.48 g, 31.362 mmol, 3 equiv). The resulting mixture was stirred for overnight at 1 10° C. The reaction was quenched with water (50 ml) at room temperature. The resulting mixture was extracted with EtOAc (3×100 mL). The combined organic layers were concentrated under reduced pressure. The residue was purified by silica gel column chromatography, eluted with PE/EA (5:1) to afford 393-3 (2.91 g, 62.57%) as a white solid.

### 4. Synthesis of 393-4

A solution of 393-3 (2.93 g, 8.122 mmol, 12 equiv) in DCE (30 mL) was treated with 244b (1.64 g, 6.768 mmol, 1.00 equiv) for 2 h at room temperature followed by the addition of STAB (2.15 g, 10.152 mmol, 1.5 equiv) at room temperature. The resulting mixture was stirred for 6 br at room temperature under nitrogen atmosphere. The reaction was quenched with sat. NaHCO₃ (aq.)(100 ml) at room temperature. The resulting mixture was extracted with EtOAc (3×50 mL). The combined organic layers were concentrated under reduced pressure. The residue was purified by reverse flash chromatography with the following conditions: column, C18 silica gel; mobile phase, MeCN in water (10 mmol/L NH₄HCO₃), 20% to 80% gradient in 30 min; detector, UV 254 nm. This resulted in 393-4 (1.7 g, 68.23%) as a white solid.

### 5. Synthesis of 393-5

A solution of 393-4 (2.7 g, 4.618 mmol, 1 equiv) in DCM (27 mL) was treated with Pyridine (2.19 g, 27.708 mmol, 6 equiv) at room temperature followed by the addition of Triphosgene (0.55 g, 1,847 mmol, 0.4 equiv) at 0° C. The solution was stirred for 30 min at room temperature under nitrogen atmosphere. The reaction was quenched by the addition of NaHCO₃ (100 mL) at room temperature. The resulting mixture was extracted with DCM/MeOH (10:1)(2×50 mL). The resulting solution was concentrated under reduced pressure. The residue was purified by reverse flash chromatography with the following conditions: column, silica gel; mobile phase, MeCN in water (0.1% NH HCO₃), 20% to 80% gradient in 30 min; detector, UV 254 nm. This resulted in 393-5 (1.83 g, 64.89%) as a yellow solid.

### 6. Synthesis of 393-6

A solution of 393-5 (1.83 g, 2.997 mmol, 1 equiv) in TEA (3.8 mL) and DCM (15.2 mL) was stirred for 2 h at room temperature under air atmosphere. The resulting mixture was concentrated under vacuum to afford 393-6 (2.7 g, NaN) as a yellow solid.

### 7. Synthesis of 393-7

A solution of 393-6 (15 g, 2.938 mmol, 1 equiv) in DCM (15 mL) was treated with methyl chloroformate (0.31 g, 3,232 mmol, 1.1 equiv) and Et₃N (0.89 g, 8.814 mmol, 3 equiv) at room temperature. The resulting mixture was stirred for 2 h at room temperature under nitrogen atmosphere. The resulting mixture was washed with 20 mL of water. The resulting mixture was extracted with CH2Cl2/MeOH (10:1) (3×20 mL). The combined organic layers were concentrated under reduced pressure. The residue was purified by Prep-TLC (CH₂Cl₂/MeOH 25:1) to afford 393-7 (392 mg, 23.47%) as a yellow solid.

### 8. Synthesis of 393-0

The 393-7 (350 mg, 0.616 mmol, 1 equiv) was purified by Chiral separation with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 50% B to 50% B in 12 min; Wave Length: 220/254 nm; RT₁ (min): 7.21; RT₂ (min): 9.46; the first peak was product) to afford 393 (117.5 mg, 32.06%) as a yellow solid.

LC-MS-393 (ES, m/z): [M+H]⁺ 569/H-NMR-393 (400 MHz, DMSO, δ ppm): 1.43-1.47 (m, 1H), 1.73-1.88 (m, 8H), 2.09-2.19 (m, 1H), 2.61-2.68 (m, 1H), 2.67-2.87 (m, 2H), 3.13-3.19 (m, 1H), 3.44 (s, 3H), 3.61 (s, 3H), 3.96-4.25 (m, 2H), 4.25-4.28 (d, 1H), 7.13 (s, 1H), 7.19-7.21 (d, 1H), 7.30 (s, 1H), 7.42-7.44 (t, 1H), 7.61 (s, 1H), 7.69-7.70 (m, 1H), 7.71 (s, 1H), 8.34 (s, 1H).

LC-MS-394: (ES, m/z): [M+H]⁺ 569. H-NMR-394: (400 MHz, DMSO, δ ppm): 1.43-1.47 (m, 1H), 1.73-1.88 (m, 8H), 2.08-2.10 (m, 1H), 2.61-2.67 (m, 1H), 2.68-2.87 (m, 2H), 3.13-3.19 (m, 1H), 3.44 (s, 3H), 3.61 (s, 3H), 3.93-4.09 (m, 2H), 4.25-4.28 (d, 1H), 7.13 (s, 1H), 7.19-7.21 (d, 1H), 7.30 (s, 1H), 7.42-7.44 (t, 1H), 7.61 (s, 1H), 7.69-7.71 (m, 1H), 7.73 (s, 1H), 8.34 (s, 1H).

### Example 391. Synthesis of Compound 394

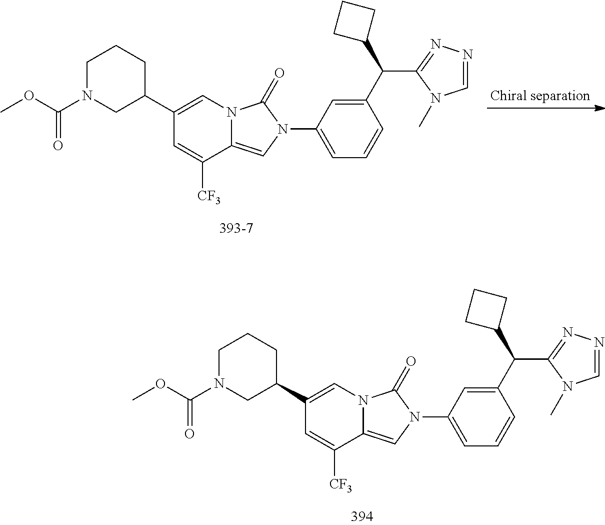 [SMILES: COC(=O)N1CCCC(c2cc(C(F)(F)F)c3cn(-c4cccc([C@H](c5nncn5C)C5CCC5)c4)c(=O)n3c2)C1 ; COC(=O)N1CCC[C@@H](c2cc(C(F)(F)F)c3cn(-c4cccc([C@H](c5nncn5C)C5CCC5)c4)c(=O)n3c2)C1]

### 1. Synthesis of 394-0

The 393-7 (350 mg) was purified by Chiral separation with the following conditions (Column: CHIRALPAK IG, 2*25 cm, 5 UM; Mobile Phase A: Hex (0.5% 2M NH₃-MeOH), Mobile Phase B: EtOH:DCM=1:1; Flow rate: 20 mL/mi; Gradient: 50% B to 50% B in 12 min; Wave Length: 220/254 nm; RT₁ (min): 7.21; RT₂ (min): 9.46; the second peak was product) to afford 394 (116 mg, 32.61%) as yellow solid.

LC-MS-394 (ES, m/z): [M+H]⁺ 569. H-NMR-394 (400 MHz, DMSO, δ ppm): 1.43-1.47 (m, 1H), 1.73-1.88 (m, 8H), 2.08-2.10 (m, 1H), 2.61-2.67 (m, 1H), 2.68-2.87 (m, 2H), 3.13-3.19 (m, 1H), 3.44 (s, 3H), 3.61 (s, 3H), 3.93-4.09 (m, 2H), 4.25-4.28 (d, 1H), 7.13 (s, 1H), 7.19-7.21 (d, 1H), 7.30 (s, 1H), 7.42-7.44 (t, 1H), 7.61 (s, 1H), 7.69-7.71 (m, 1H), 7.73 (s, 1H), 8.34 (s, 1H).

### Example 392. Synthesis of Compound 395

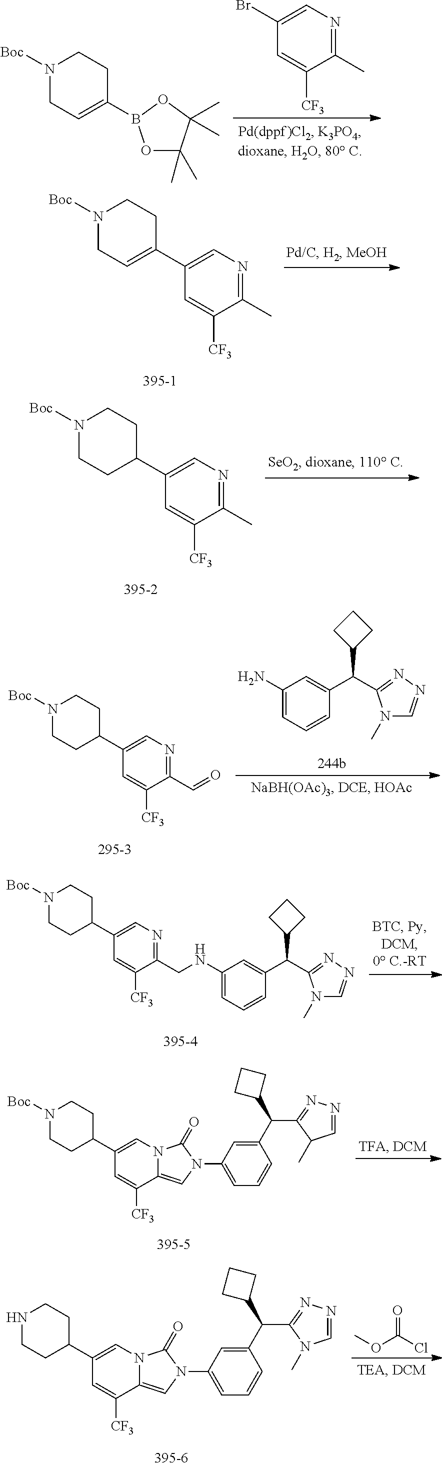

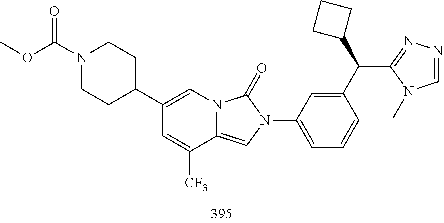 [SMILES: COC(=O)N1CCC(c2cc(C(F)(F)F)c3cn(-c4cccc([C@H](c5nncn5C)C5CCC5)c4)c(=O)n3c2)CC1]

### 1. Synthesis of 395-1

A solution of tert-butyl 4-(4,4,5,5-tetramethyl-1,3,2-dioxaborolan-2-yl)-3,6-dihydro-2H-pyridine-1-carboxylate (4 g, 12,936 mmol, 1 equiv) and 5-bromo-2-methyl-3-(trifluoromethyl) pyridine (3.10 g, 12,936 mmol, 1 equiv) in dioxane (32 mL) and 1120 (8 mL) was added K₃PO₄ (8.24 g, 38.808 mmol, 3 equiv) and Pd(dppf)Cl₂ (1.42 g, 1.940 mmol, 0.15 equiv) at room temperature under nitrogen atmosphere. The reaction was quenched with Water (30 mL) at room temperature. The aqueous layer was extracted with CH₂Cl₂ (3×20 mL). The resulting mixture was concentrated under reduced pressure. The residue was purified by silica gel column chromatography, eluted with PE/EA (20:1) to afford 395-1 (4 g, 83.99%) as a yellow solid.

### 2. Synthesis of 395-2

To a solution of 395-1 (4 g, 11.684 mmol, 1 equiv) in 40 mL MeOH was added Pd/C (10%, 400 mg) under nitrogen atmosphere in a 250 mL round-bottom flask. The mixture was hydrogenated at room temperature for 4 h under hydrogen atmosphere using a hydrogen balloon, filtered through a Celite pad and concentrated under reduced pressure. The resulting mixture was filtered, the filter cake was washed with MeOH (3×10 mL). The filtrate was concentrated under reduced pressure. This resulted 395-2 (3.5 g, 80.90%) as a yellow solid.

### 3. Synthesis of 395-3

A solution of 395-2 and SeO₂ (4.51 g, 40.652 mmol, 4 equiv) in dioxane (35 mL) was stirred for overnight at 110° C. under nitrogen atmosphere. The reaction was quenched with Water (50 mL) at room temperature. The aqueous layer was extracted with EtOAc (3×50 mL). The resulting mixture was concentrated under reduced pressure. The residue was purified by silica gel column chromatography, eluted with PE/EA (5:1) to afford 395-3 (1.5 g, 38.71%) as a yellow solid.

### 4. Synthesis of 395-4

Into a 100 mL round-bottom flask were added 395-3 (250 mg, 0.698 mmol, 1 equiv) 244b (202.86 mg, 0.838 mmol, 1.2 equiv), NaBH(OAc)3 (443.56 mg, 2,094 mmol, 3 equiv) and DCE (4 mL) at room temperature. The resulting mixture was stirred for overnight at room temperature. The reaction was quenched with Water (10 mL) at room temperature. The aqueous layer was extracted with CH2Cl2 (3×10 mL). The resulting mixture was concentrated under reduced pressure. The residue was purified by Prep-TLC (CH₂Cl₂/MeOH 15:1) to afford 395-4 (230 mg, 51.21%) as a yellow solid.

### 5. Synthesis of 395-5

The solution of compound 395-4 (1.4 g, 2.394 mmol, 1 equiv) and Pyridine (1.14 g, 14.364 mmol, 6 equiv) in DCM (20 mL) was stirred for 5 min at 0° C. Into the mixture was added Triphosgene (0.46 g, 1.556 mmol, 0.65 equiv) at 0° C. The resulting mixture was stirred for 10 min at 0° C. The reaction was quenched with sat. NaHCO₃ (aq.) (30 ml). The aqueous layer was extracted with CH₂Cl₂ (3×10 mL). The resulting mixture was concentrated under reduced pressure. The residue was purified by Prep-TLC (CH₂Cl₂/MeOH 15:1) to afford 395-5 (700 mg, 45.48%) as a yellow solid.

### 6. Synthesis of 395-6

Into a 8 mL sealed tube were added 395-5 (700 mg, 1.146 mmol, 1 equiv) TEA (2 mL) and DCM (4 mL) at room temperature. The resulting mixture was stirred for 2 h at room temperature. The mixture/residue was acidified to pH 7 with saturated NaHCO₃ (aq.) (10 mL). The aqueous layer was extracted with CH₂Cl₂ (3×10 ml). The resulting mixture was concentrated under reduced pressure. The residue was purified by Prep-TLC (CH₂Cl₂/MeOH 10:1) to afford 395-6 (350 mg, 56.81%) as a yellow solid.

### 7. Synthesis of 395

A solution of 395-6 (150 mg, 0,294 mmol, 1 equiv) and TEA (89.19 mg, 0.882 mmol, 3 equiv) in DCM (2 mL) was stirred for 5 min at 0° C. The resulting mixture was added methyl chloroformate (30.54 mg, 0.323 mmol, 1.1 equiv) and stirred for 2.5 h at room temperature. The reaction was quenched with sat. NaHCO₃ (aq.) (5 mL) at room temperature. The aqueous layer was extracted with CH₂Cl₂ (3×10 mL). The residue was purified by Prep-TLC (CH₂Cl₂/MeOH 10:1) to afford the crude product. The crude product (90 mg) was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 36% B to 51% B in 8 min; Wave Length: 220 nm; RT1 (min): 7.9) to afford 395 (55.5 mg, 33.02%) as a yellow solid.

LC-MS-395: (ES, m/z): [M+H]⁺ 569. H-NMR: (400 MHz, DMSO-d6, δ ppm): 1.48-1.52 (m, 2H), 1.75-1.80 (m, 7H), 2.08-2.15 (s, 1H), 2.57-2.61 (m, 1H), 2.71-3.06 (s, 2H), 3.11-3.30 (s, 1H), 3.40-3.52 (s, 3H), 3.58-3.71 (s, 3H), 4.05-4.21 (d, 1H), 7.12-7.26 (m, 2H), 7.27-7.39 (s, 1H), 7.40-7.50 (m, 1H), 7.50-7.58 (s, 1H), 7.61-7.73 (d, 1H), 7.73-7.80 (d, 1H), 8.19-8.48 (s, 1H).

### Example 393. Synthesis of Compound 396 and 397

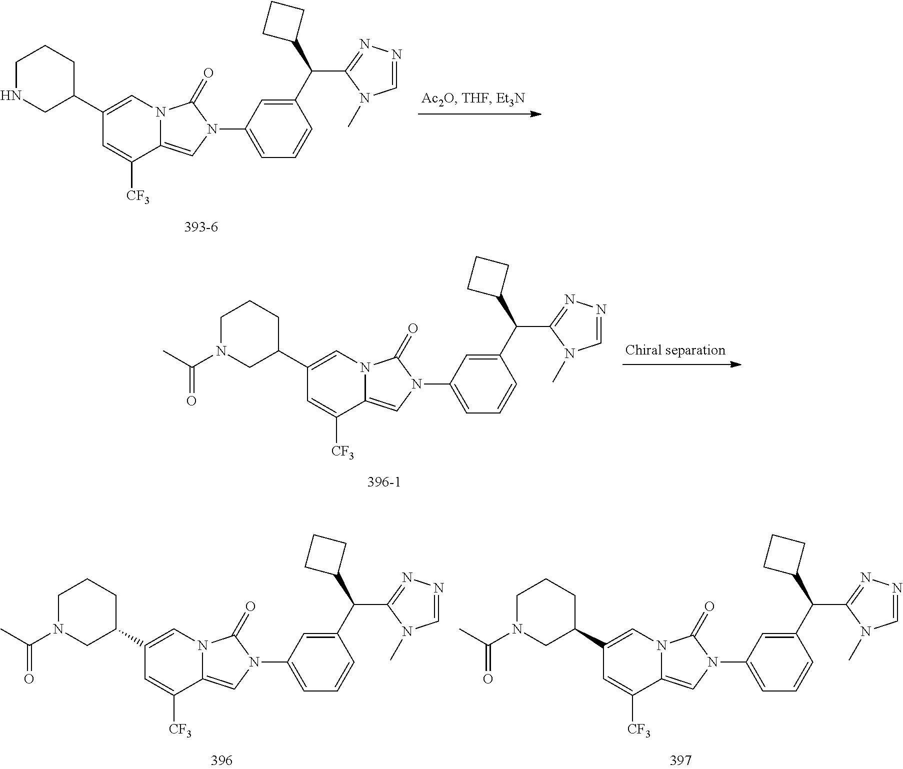

### 1. Synthesis of 396-1

A solution of 393-6 (1.2 g, 2.350 mmol, 1 equiv) in THF (12 mL) was treated with acetic anhydride (0.36 g, 3.525 mmol, 1.5 equiv) and Et₃N (0.71 g, 7.050 mmol, 3 equiv) at room temperature. The resulting mixture was stirred for 2 h at room temperature under nitrogen atmosphere. The reaction was quenched with water (100 mL) at room temperature. The resulting mixture was extracted with EtOAc (3×200 mL). The combined organic layers were concentrated under reduced pressure. The residue was purified by reverse flash chromatography with the following conditions: column, C18 silica gel; mobile phase, MeCN in Water (0.1% NH₄HCO₃), 20% to 80% gradient in 30 min; detector, UV 254 nm to afford 396-1 (531 mg, 38.84%) as a yellow solid.

### 2. Synthesis of 396 and 397

The 396-1 (500 mg) was purified by Chiral separation with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 55% B to 55% B in 12 min; Wave Length: 220/254 nm; RT₁ (min): 7.566; RT₂ (min): 9.486; the first peak was 396-0; the second peak 397) to afford 396 (162.3 mg, 31.10%) as a light yellow solid and 397 (191.5 mg, 37.50%) as a light yellow solid.

LC-MS-396 (ES, m/z): [M+H]⁺ 553. H-NMR-396 (400 MHz, DMSO, δ ppm): 1.33-1.53 (m, 1H), 1.68-1.87 (m, 9H), 2.03-2.08 (m, 4H), 2.50-2.61 (m, 1H), 3.01-3.27 (m, 2H), 3.44 (s, 3H), 3.71-3.84 (d, 1H), 4.25-4.28 (d, 1H), 4.31-4.44 (t, 1H), 7.11-7.32 (m, 3H), 7.45-7.46 (t, 1H), 7.59-7.74 (m, 3H), 8.34 (s, 1H).

LC-MS-397 (ES, m/z): [M+H]⁺ 553. H-NMR-397: (400 MHz, DMSO, δ ppm): 1.33-1.53 (m, 1H), 1.68-1.87 (m, 9H), 2.03-2.08 (m, 4H), 2.62-2.72 (m, 1H), 3.01-3.27 (m, 2H), 3.44 (s, 3H), 3.78-3.81 (m, 1H), 4.26-4.28 (d, 1H), 4.33-4.41 (t, 1H), 7.11-7.22 (m, 2H), 7.32 (s, 1H), 7.43-7.46 (t, 1H), 7.60-7.74 (m, 3H), 8.34 (s, 1H).

### Example 394. Synthesis of Compound 398-0

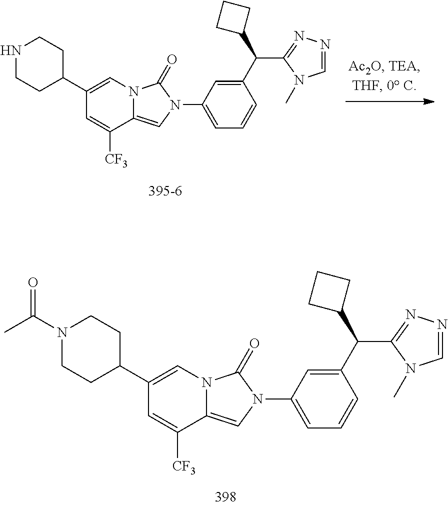 [SMILES: Cn1cnnc1[C@@H](c1cccc(-n2cc3c(C(F)(F)F)cc(C4CCNCC4)cn3c2=O)c1)C1CCC1 ; CC(=O)N1CCC(c2cc(C(F)(F)F)c3cn(-c4cccc([C@H](c5nncn5C)C5CCC5)c4)c(=O)n3c2)CC1]

### 1. Synthesis of 398

A solution of 395-6 (150 mg, 0,294 mmol, 1 equiv) and Ac₂O (44.99 mg, 0,441 mmol, 1.5 equiv) in THF (2 mL) was stirred for 5 min at 0° C. The resulting mixture was added TEA (89.19 mg, 0.882 mmol, 3 equiv) and stirred for 2.5 h at room temperature. The reaction was quenched with sat. NaHCO₃ (aq.) (10 mL) at room temperature. The aqueous layer was extracted with CH₂Cl₂ (3×10 mL). The residue was purified by Prep-TLC (CH₂Cl₂/MeOH 10:1) to afford the crude product. The crude product (90 mg) was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 36% B to 51% B in 8 min; Wave Length: 254 nm; RT1 (min): 7.9 to afford 398 (52.0 mg, 31.77%) as a yellow solid.

LC-MS-398: (ES, m/z): [M+H]⁺ 553. H-NMR-398: (400 MHz, DMSO, δ ppm): 1.32-1.49 (m, 1H), 1.55-1.68 (m, 1H), 1.68-1.94 (m, 7H), 1.97-2.07 (s, 3H), 2.07-2.15 (s, 1H), 2.52-2.61 (s, 1H), 2.62-2.75 (m, 1H), 3.02-3.16 (m, 1H), 3.16-3.27 (s, 1H), 3.41-3.49 (s, 3H), 3.83-4.00 (d, 1H), 4.18-4.38 (d, 1H), 4.42-4.69 (d, 1H), 7.10-7.26 (m, 2H), 7.26-7.39 (s, 1H), 7.39-7.50 (m, 1H), 7.50-7.67 (s, 1H), 7.70-7.87 (m, 2H), 8.25-8.41 (s, 1H).

### Example 395. Synthesis of Compound 399 and 400

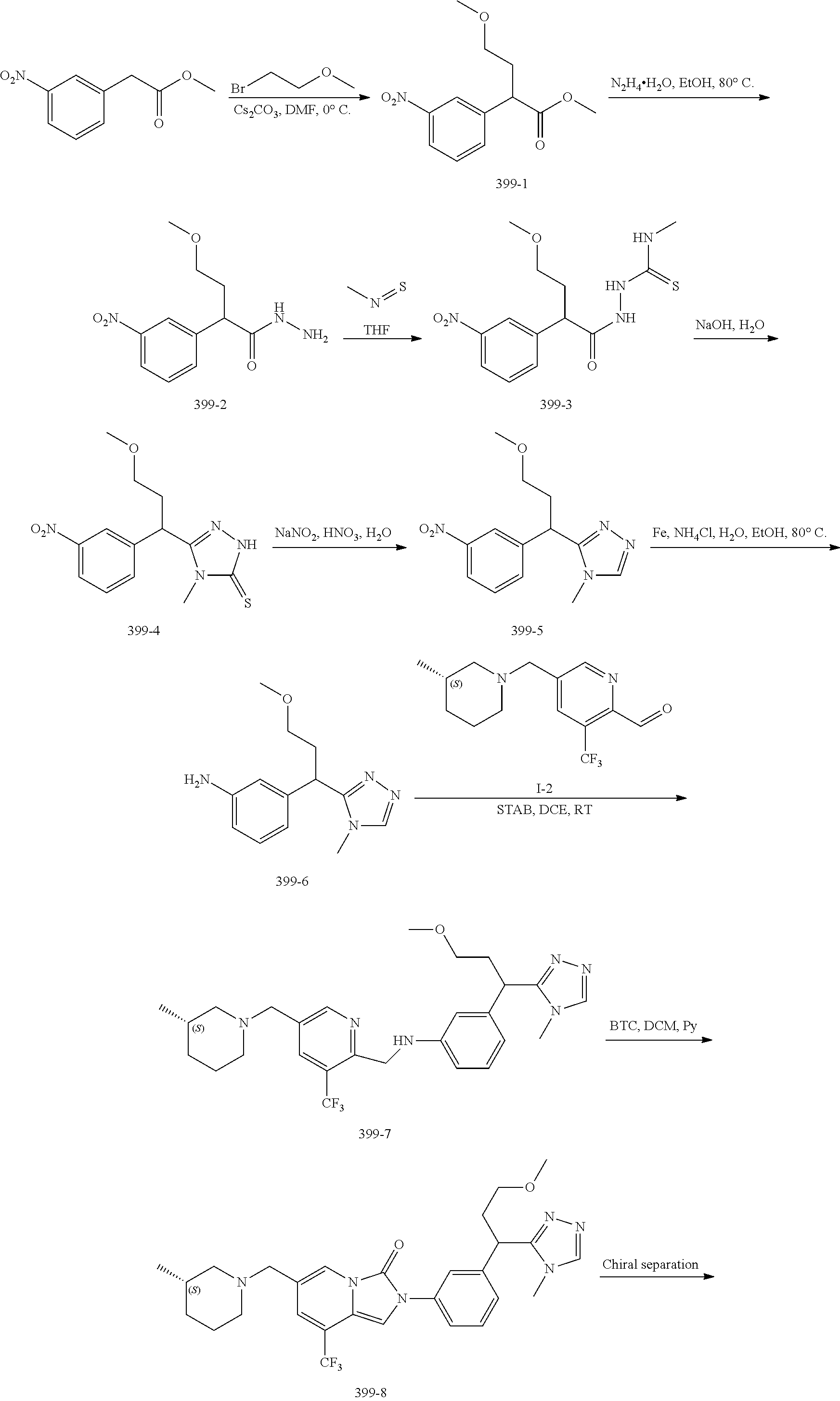

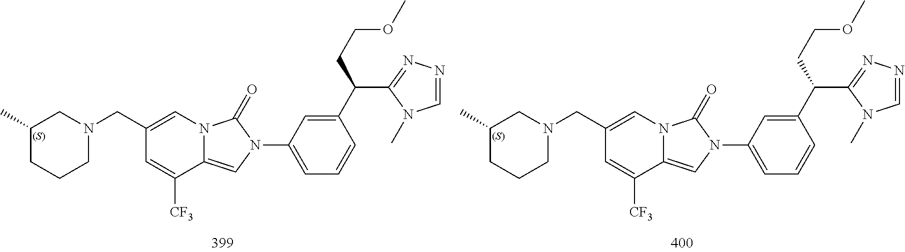

### 1. Synthesis of 399-1

A solution of methyl 2-(3-nitrophenyl) acetate (10 g, 51.236 mmol, 1 equiv) and Cs2CO3 (83.47 g, 256.180 mmol, 5 equiv) in DMF (100 mL) was stirred for 3 h at 0° C. under nitrogen atmosphere. To the above mixture was added 2-bromoethyl methyl ether (21.36 g, 153.708 mmol, 3 equiv) room temperature. The resulting mixture was stirred overnight at room temperature. The reaction was quenched with saturated NH4Cl (aq.) (300 mL) at room temperature. The aqueous layer was extracted with EtOAc (3×200 mL). The resulting mixture was concentrated under reduced pressure. The residue was purified by silica gel column chromatography, eluted with PE/EA (100:1) to afford 399-1 (7 g, 48.55%) as a yellow oil.

### 2. Synthesis of 399-2

A solution of 399-1 (6.9 g, 27.245 mmol, 1 equiv) and NH₂NH₂·H2O (8.73 g, 272.450 mmol, 10 equiv) in EtOH (70 mL) was stirred overnight at 80° C. The reaction was quenched with saturated NH_{A}Cl (aq.) (200 mL) at room temperature. The precipitated solids were collected by filtration and washed with water (3×50 mL). This resulted in 399-2 (6.8 g, 88.69%) as a yellow solid.

### 3. Synthesis of 399-3

A solution of 399-2 (6.8 g, 26.850 mmol, 1 equiv) and methyl isothiocyanate (2.94 g, 40.275 mmol, 1.5 equiv) in tetrahydrofuran (80 mL) was stirred for 5b at room temperature. The resulting mixture was washed with water (40 mL). The reaction was diluted with saturated water (100 mL) at room temperature. The precipitated solids were collected by filtration and washed with water (3×40 mL). This resulted in 399-3 (8.2 g, 84.22%) as a yellow solid.

### 4. Synthesis of 399-4

A solution of 399-3 (8.7 g, 26.657 mmol, 1 equiv) and NaOH (10.66 g, 266,570 mmol, 10 equiv) in H2O (200 mL) was stirred for 5 at room temperature under air atmosphere. The mixture was neutralized to pH 6 with HCl (aq.). The precipitated solids were collected by filtration and washed with water (2×30 mL). This resulted in 399-4 (8.2 g, 89.78%) as a yellow solid.

### 5. Synthesis of 399-5

To a stirred solution of 399-4 (8.2 g, 26.592 mmol, 1 equiv) and NaNO₂ (18.35 g, 265.920 mmol, 10 equiv) in H2O (82 ml) was added HNO₃ (265.92 mL, 265.920 mmol, 10 equiv, 1M) dropwise at at room temperature. The result mixture was stirred for 5 h at room temperature. The mixture was neutralized to pH 7 with saturated NaHCO₃ (aq.) (300 mL). The aqueous layer was extracted with EtOAc (3×100 mL). The resulting mixture was concentrated under reduced pressure. The residue was purified by silica gel column chromatography, eluted with CH2Cl₂/MeOH (80:1) to afford 399-5 (8 g, 97.99%) as a yellow solid.

### 6. Synthesis of 399-6

To a stirred solution of 399-5 (4 g, 14.477 mmol, 1 equiv) and Fe (2.43 g, 43.431 mmol, 3 equiv) in EtOH (40 mL) were added NH4Cl (7.74 g, 144,770 mmol, 10 equiv) in H2O (40 mL) at room temperature. The resulting mixture was stirred for 3 h at 80° C. The resulting mixture was filtered, the filter cake was washed with EtOAc (2×50 mL). The resulting mixture was diluted with water (100 mL). The aqueous layer was extracted with EtOAc (3×50 mL). The resulting mixture was concentrated under reduced pressure. The residue was purified by silica gel column chromatography, eluted with DCM/MeOH (20:1) to afford 399-6 (2 g, 50.48%) as a brown solid,

### 7. Synthesis of 399-7

A solution of 399-6 (600 mg, 2.436 mmol, 1 equiv) and 1-2 (767.14 mg, 2.680 mmol, 1.1 equiv) and STAB (1032.54 mg, 4.872 mmol, 2 equiv) in DCE (10 mL) was stirred for 5 h at room temperature. The mixture was quenched with saturated NaHCO₃ (aq.) (80 mL). The aqueous layer was extracted with CH₂Cl₂ (3×50 mL). The resulting mixture was concentrated under reduced pressure. The residue was purified by Prep-TLC (CH₂Cl₂/MeOH 20:1) to afford 399-7 (900 mg, 67.94%) as a yellow solid,

### 8. Synthesis of 399-8

To a stirred solution of 399-7 (600 mg 1.161 mmol, 1 equiv) and Pyridine (551.20 mg, 6.966 mmol, 6 equiv) in DCM (15 ml) was added Triphosgene (127.52 mg, 0.430 mmol, 0.37 equiv) at 0° C. The result mixture was stirred for 10 min at 0° C. The mixture was quenched with saturated NaHCO₃ (aq.) (50 mL). The aqueous layer was extracted with CH₂Cl₂ (3×30 mL). The resulting mixture was concentrated under reduced pressure. The residue was purified by Prep-TLC (CH2Cl₂/MeOH 20:1) to afford 399-8 (400 mg, 60.30%) as a yellow solid.

### 9. Synthesis of 399

The 399-8 (400 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 ml/Ain; Gradient: 70% B to 70% B in 12.5 min; Wave Length: 220/254 nm; RT1 (min): 4.96; RT2 (min): 8.87; the first peak is 400; the second peak is 399) to afford 400 (155 mg, 38.47%) as a yellow solid and 399 (139.1r mg, 34.33%) as a yellow solid.

LC-MS-400 (ES, m/z): [M+H]⁺ 543. H-NMR-400 (400 MHz, DMSO-d6, δ ppm): 0.86-0.97 (d, 4H), 1.41-1.51 (m, 1H), 1.51-1.71 (m, 4H), 1.87-1.92 (m, 1H), 2.20-2.34 (m, 1H), 2.47-2.50 (m, 1H), 2.50-2.52 (m, 1H), 2.68-2.75 (m, 2H), 3.21 (s, 3H), 3.27-3.29 (m, 3H), 3.43 (s, 3H), 4.36-4.40 (m, 1H), 7.00 (s, 1H), 7.18-7.20 (d, 1H), 7.34 (s, 1H), 7.44-7.48 (m, 1H), 7.65 (s, 1H), 7.71-7.75 (m, 2H), 8.36 (s, 1H). LC-MS-399 (ES, m/z): [M+H]⁺ 543

H-NMR-399 (400 MHz, DMSO-d6, δ ppm): 0.86-0.97 (d, 4H), 1.41-1.71 (m, 5H), 1.87-1.92 (m, 1H), 2.20-2.34 (m, 1H), 2.48-2.51 (m, 2H), 2.68-2.75 (m, 2H), 3.21 (s, 3H), 3.27-3.29 (m, 3H), 3.43 (s, 3H), 4.36-4.40 (m, 1H), 7.00 (s, 1H), 7.18-7.20 (d, 1H), 7.34 (s, 1H), 7.44-7.48 (m, 1H), 7.65 (s, 1H), 7.72-7.75 (m, 2H), 8.35 (s, 1H).

### Example 396. Synthesis of Compound 401_P1&P2

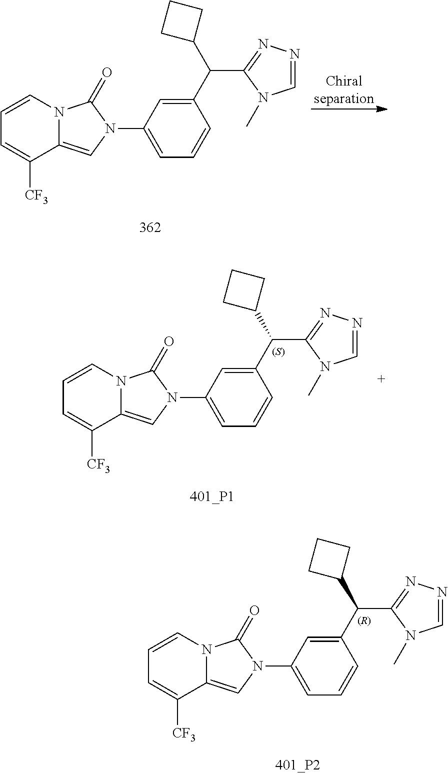

### 1. Synthesis of 401_P1, P2

The crude 362 was purified by prep-HPLC (column: Phenomenex C18 75*30 mm*3 um; Mobile phase A: water (NH₃H2O+NH₄HCO₃), Mobile phase B: ACN; Flow rate: 70 mL/min; Gradient: 20% B to 50% B in 8 min; Wave Length: 220 nm) to afford pure 362 (30 mag) as a yellow solid. 362 was purified by Chiral separation with the following conditions (column: DAICEL CHIRALCEL OD (250 mm*30 mm, 10 um); Mobile phase: [Neu-IPA]; B %: 50%-50%, 15 min, Flow rate: 70 mL/min; Wave Length: 220/254 nm; RT1 (min): 7.85; RT2 (min): 10.1) to afford 401 P1 (6.1 mg, 19.3%) and 401_P2 (7.7 mg, 24.6%).

MS-401_P1: (ES, m/z): [M+H]⁺ 428.17. H-NMR-401_P1: (400 MHz, DMSO-d6) δ 8.34 (s, 1H), 7.80-7.77 (m, 2H), 7.71-7.69 (m, 1H), 7.45-7.31 (m, 1H), 7.21 (s, 1H), 7.19-7.10 (m, 1H), 7.12-7.05 (m, 1H), 6.31-6.28 (m, 1H), 4.29-4.26 (m, 1H), 3.44-3.41 (m, 3H), 3.27-3.22 (m, 1H), 2.09 (s, 1H), 1.82-1.69 (m, 5H).

MS-401_P2: (ES, m/z): [M+H]⁺ 428.17. H-NMR-401_P2: (400 MHz, DMSO-d6) δ 8.34 (s, 1H), 7.80-7.76 (m, 2H), 7.76-7.69 (m, 1H), 7.44 (t, J=8.0 Hz, 1H), 7.33-7.28 (m, 1H), 7.22-7.17 (m, 1H), 7.12-7.07 (m, 1H), 6.31-6.28 (m, 1H), 4.26 (d, J=10.0 Hz, 1H), 3.46-3.39 (m, 3H), 3.25-3.22 (m, 1H), 2.14-2.04 (m, 1H), 1.87-1.66 (m, 5H).

### Example 397. Synthesis of Compound 402

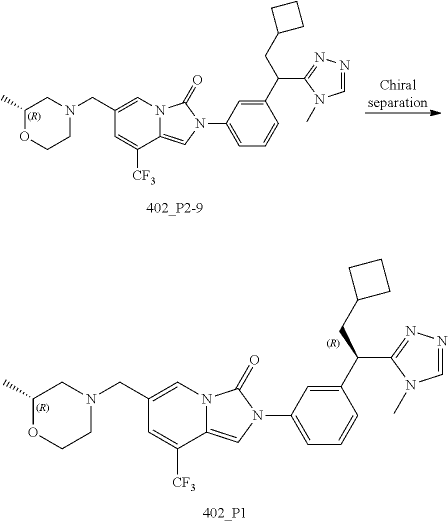

### 1. Synthesis of 402

The 402_P2-9 (68.7 mg) was purified by Chiral separation with the following conditions (column: DAICEL CHIRALCEL OD (250 mm*30 mm, 10 um); mobile phase: [Neu-IPA]; B %: 50%-50%, 10 min, Flow rate: 60 mL/min; Wave Length: 220/254 nm; RT1 (min): 4.71; RT2 (min): 6.03) to afford 402 (24 mg, 34.44%) as a yellow solid.

MS-402: (ES, m/z): [M+H]⁺ 555.2. ¹H-NMR-402: (400 MHz, METHANOL-d4) δ 8.35 (s, 1H), 7.67 (d, J=4.4 Hz, 2H), 7.65-7.61 (m, 1H), 7.49 (t, J=8.0 Hz, 1H), 7.26 (d, J=7.6 Hz, 1H), 7.11 (d, J=14.8 Hz, 2H), 4.20 (t, J=7.6 Hz, 1H), 3.87-3.82 (m, 1H), 3.69-3.62 (m, 2H), 3.52 (s, 3H), 3.33 (s, 2H), 2.80 (d, J=11.6 Hz, 1H), 2.74 (d, J=11.6 Hz, 1H), 2.46-2.37 (m, 1H), 2.32-2.23 (m, 2H), 2.21-2.14 (m, 1H), 2.10-2.03 (m, 1H), 1.94-1.89 (m, 11H), 1.87-1.78 (m, 3H), 1.76-1.64 (m, 2H), 1.13 (d, J=6.4 Hz, 3H).

### Example 398. Synthesis of Compound 403 P1&P2

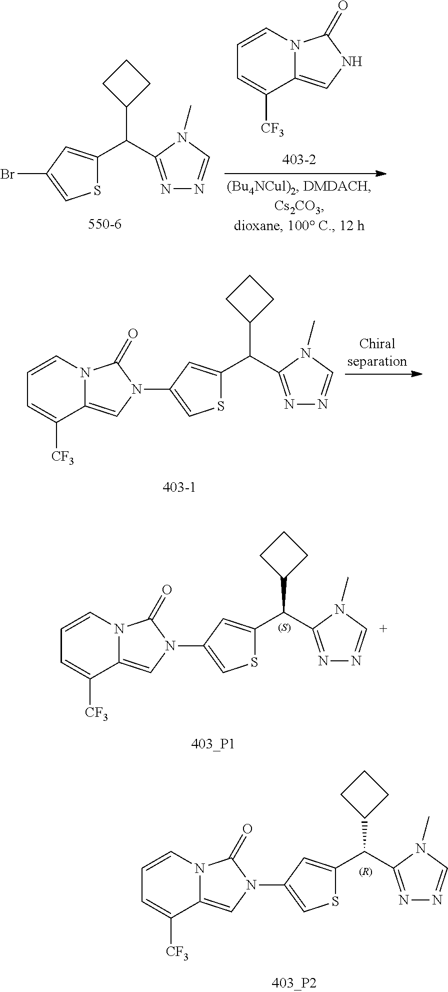

### 1. Synthesis of 403-1

To a solution of 550-6 (0.18 g, 576.50 umol, 1 equiv) and 403-2 (128 mg, 634 umol, 1.1 equiv) in dioxane (1 mL) was added iodocopper;tetrabutylammonium;diiodide (64.5 mg, 57.6 umol, 0.1 equiv), (1R,2R)—N1,N2-dimethylcyclohexane-1,2-diamine (16.4 mg, 115 umol, 0.2 equiv) and Cs2CO3 (375 mg, 1.15 mmol, 2 equiv) under nitrogen atmosphere, the mixture was stirred at 110° C. for 12 h under nitrogen atmosphere. The reaction mixture was diluted with water (10 mL), extracted with EtOAc (10 mL×2). The combined organic layers were dried over anhydrous Na2SO₄, filtered and concentrated in vacuum. The residue was purified by silica gel column chromatography eluted with CH₂Cl₂/MeOH (10:1) to give crude product. The crude product (140 mg) was purified by Prep-HPLC with the following conditions (Column: Phenomenex Luna C18 75*30 mm*3 um; Mobile Phase A: Water (0.2% formic acid), Mobile Phase B: ACN; Flow rate: 25 mL/min; Gradient: 40% B to 70% B in 8 min; Wave Length: 220 nm; RT1 (min): 6.5) afford 403-1 (25 mg, 8.3%) as a yellow solid.

### 2. Synthesis of 403_P1&P2

The 403-1 (25 mg) was purified by Chiral separation with the following conditions (column: DAICEL CHIRALCEL OD (250 mm*30 nm, 10 um); Mobile Phase: [IA]; B %: 50%-50%, 10 min, Flow rate: 27 mL/min; Wave Length: 220/254 am; RT1 (min): 5.77; RT2 (min): 6.50) to afford 403_P1 (4 mg 15%) and 403_P2 (8 mg, 30%) as yellow solid.

MS-403_P1: (ES, m/z): [M+H]⁺ 434.0. H-NMR-403_P1: (400 MHz, CHLOROFORM-d) δ 8.07 (s, 1H), 7.69 (d, J=7.2 Hz, 1H), 7.58 (d, J=1.6 Hz, 1H), 7.24 (d, J=1.6 Hz, 1H), 6.86 (d, J=6.8 Hz, 1H), 6.76 (s, 1H), 6.15 (t, J=6.8 Hz, 1H), 4.24 (d, J=10.4 Hz, 1H), 3.54 (s, 3H), 3.41-3.30 (m, 1H), 2.30-2.20 (m, 1H), 2.17-2.10 (m, 1H), 1.98-1.82 (m, 3H), 1.79-1.71 (m, 1H)

MS-403_P2: (ES, m/z): [M+H]⁺ 434.1. H-NMR-403_P2: (400 MHz, CHLOROFORM-d) δ 8.10 (s, 1H), 7.73 (d, J=7.6 Hz, 1H), 7.61 (d, J=1.6 Hz, 1H), 7.27 (d, J=0.8 Hz, 1H), 6.89 (d, J=6.8 Hz, 1H), 6.79 (s, 1H), 6.19 (t, J=6.8 Hz, 1H), 4.27 (d, J=10.4 Hz, 1H), 3.57 (s, 3H), 3.47-3.32 (m, 1H), 2.34-2.24 (m, 1H), 2.22-2.13 (m, 1H), 2.03-1.86 (m, 3H), 1.81-1.74 (m, 1H).

### Example 399. Synthesis of Compound 404_P1 & P2

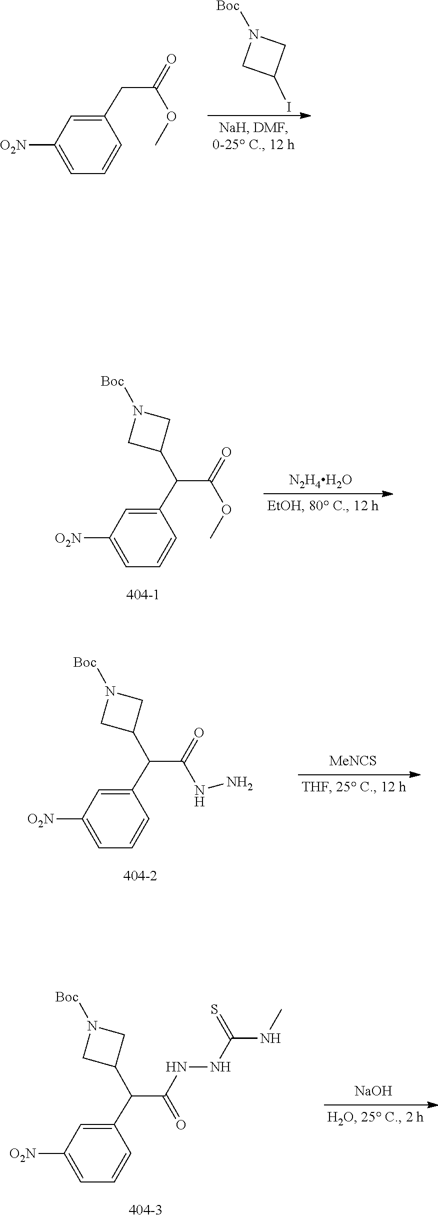

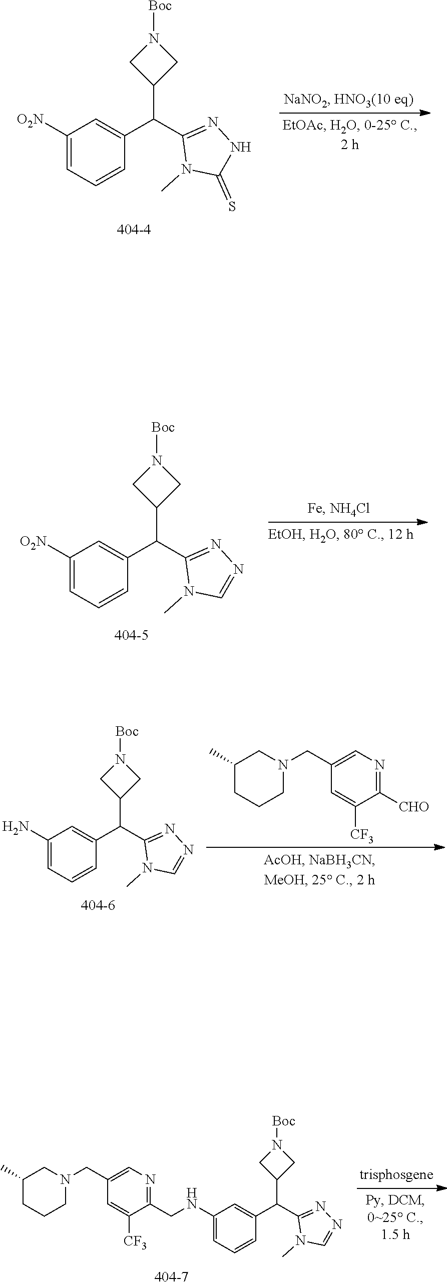

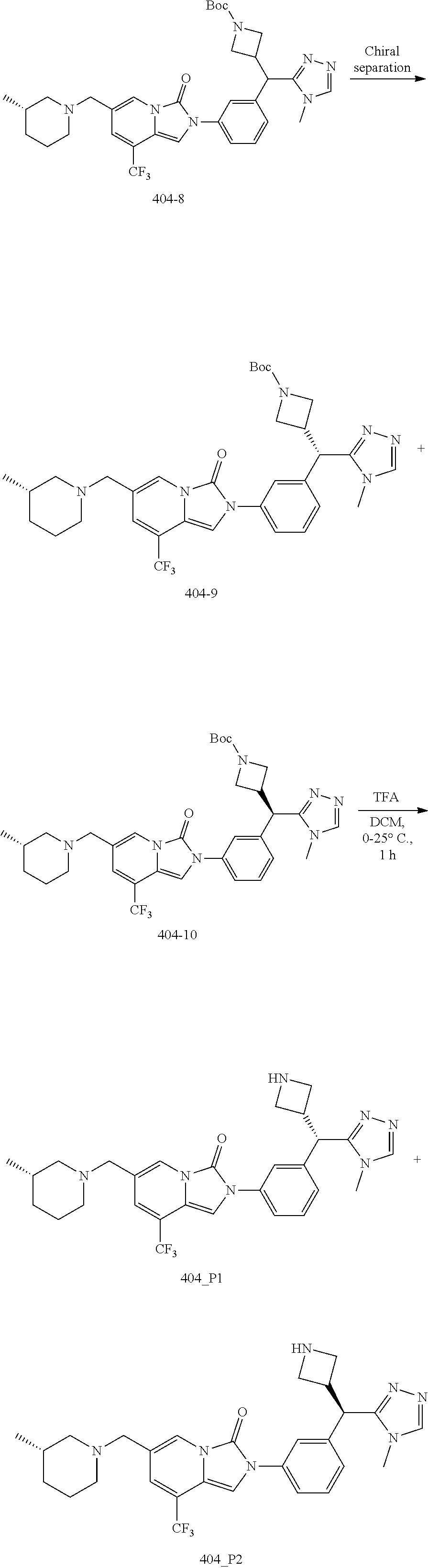 [SMILES: C[C@H]1CCCN(Cc2cc(C(F)(F)F)c3cn(-c4cccc(C(c5nncn5C)C5CN(C(=O)OC(C)(C)C)C5)c4)c(=O)n3c2)C1 ; C[C@H]1CCCN(Cc2cc(C(F)(F)F)c3cn(-c4cccc([C@@H](c5nncn5C)C5CN(C(=O)OC(C)(C)C)C5)c4)c(=O)n3c2)C1 ; "TFA" ; C[C@H]1CCCN(Cc2cc(C(F)(F)F)c3cn(-c4cccc([C@H](c5nncn5C)C5CN(C(=O)OC(C)(C)C)C5)c4)c(=O)n3c2)C1 ; C[C@H]1CCCN(Cc2cc(C(F)(F)F)c3cn(-c4cccc([C@@H](c5nncn5C)C5CNC5)c4)c(=O)n3c2)C1 ; C[C@H]1CCCN(Cc2cc(C(F)(F)F)c3cn(-c4cccc([C@H](c5nncn5C)C5CNC5)c4)c(=O)n3c2)C1]

### 1. Synthesis of 404-1

To a solution of NaH (2.07 g, 51.8 mmol, 60% purity, 1.1 equiv) in DMF (90 mL) was added methyl 2-(3-nitrophenyl)acetate (9.2 g, 47.1 mmol, 1 equiv). The mixture was cooled to 0° C., then tert-butyl 3-iodoazetidine-1-carboxylate (22.6 g, 80.1 mmol, 1.7 equiv) was added at 0° C. The mixture was stirred at 25° C. for 12 h under nitrogen atmosphere. The reaction mixture was quenched by Sat.NH4Cl (800 mL), extracted with EtOAc (250 mL×3). The combined organic layers were dried over anhydrous Na2SO4, filtered and the filtrate was concentrated in vacuum. The residue was purified by silica gel column chromatography eluted with PE/EtOAc (5:1) to afford 404-1 (13 g, 78%) as a yellow solid.

### 2. Synthesis of 404-2

To a solution of 404-1 (13 g, 37.1 mmol, 1 equiv) in EtOH (80 mL) was added N2H4.1H2O (17.1 mL, 346 mmol, 98% purity, 9.4 equiv). The mixture was stirred at 80° C. for 12 h. The reaction mixture was concentrate in vacuum to remove EtOH (80 mL), then the mixture was diluted with water (800 mL), and extracted with EtOAc (200 mL×3). The combined organic layers were dried over anhydrous Na2SO4, filtered and the filtrate was concentrated in vacuum to afford 404-2 (12.5 g, crude) as a yellow solid.

### 3. Synthesis of 404-3

To a solution of 404-2 (12.5 g, 12.96 mmol, 1 equiv) in THF (250 mL) was added methylimino(thioxo)methane (4.88 mL, 71.3 mmol, 2 equiv). The mixture was stirred at 25° C. for 4 hr. The reaction mixture was diluted water (800 mL), extracted with EtOAc (350 mL×2). The combined organic layers were dried over anhydrous Na2SO4, filtered and concentrated in vacuum to afford 404-3 (15 g, crude) as a brown solid,

11H-NMR-404-3: (400 MHz, DMSO-d6) δ 10.04 (s, 1H), 9.21 (s, 1H), 8.22 (s, 1H), 8.18-8.13 (m, 1H), 7.90 (s, 1H), 7.81 (d, J=7.6 Hz, 1H), 7.65 (t, J=8.0 Hz, 1H), 4.61 (s, 1H), 4.11-4.07 (m, 1H), 3.98-3.87 (m, 3H), 3.69 (d, J=16.0 Hz, 1H), 3.17 (d, J=5.2 Hz, 1H), 2.92-2.80 (m, 3H), 1.36 (s, 9H)

### 4. Synthesis of 404-4

To a solution of NaOH (22.6 g, 566 mmol, 16 equiv) in water (220 mL) was added 404-3 (15 g, 35.4 mmol, 1 equiv). The mixture was stirred at 25° C. for 2 hr. The reaction mixture was adjusted pH to 3 by 1N HCl. Then the mixture was filtered and the filter cake was concentrated in vacuum to afford 404-4 (14.7 g, crude) as a brown solid.

1H-NMR-404-4: (400 MHz, DMSO-d6) δ 13.80 (s, 1H), 8.20-8.15 (m, 2H), 7.77 (d, J=7.6 Hz, 1H), 7.72-7.64 (m, 1H), 4.84 (d, J=11.2 Hz, 1H), 4.61 (s, 1H), 3.97 (s, 1H), 3.65 (s, 3H), 3.17 (s, 3H), 1.35 (s, 9H)

### 5. Synthesis of 404-5

HNO3 (8.16 mL, 123 mmol, 68% purity, 10 equiv) was added to water (124 mL) to afford the diluted HNO3 solution (1 M, 132 mL). To a solution of 404-4 (5 g, 12.3 mmol, 1 equiv) and NaNO2 (8.51 g, 123 mmol, 10 equiv) in EtOAc (20 mL) and water (100 mL) was added diluted HNO3 solution (1 M, 132 mL) dropwise at 0° C. Then the mixture was stirred at 25° C. for 2 hr. The reaction was quenched by the addition of Sat.NaHCO₃ (800 mL). The aqueous phase was extracted with EtOAc (200 mL×3). The combined organic layers were dried over anhydrous Na2SO4, filtered and the filtrate was concentrated in vacuum to afford 404-5 (3.55 g, crude) as a yellow oil.

1H-NMR-404-5: (400 MHz, CHLOROFORM-d) δ 8.13-8.07 (m, 2H), 8.03 (s, 1H), 7.56-7.45 (m, 2H), 4.24-4.19 (m, 2H), 3.85-3.74 (m, 1H), 3.63-3.57 (m, 1H), 3.56-3.44 (m, 2H), 3.37 (s, 3H), 1.35 (s, 9H)

### 6. Synthesis of 404-6

To a solution of 404-5 (3.55 g, 9.51 mmol, 1 equiv) in EtOH (50 mL) was added Fe (2.65 g, 47.5 mmol, 5 equiv) and NH4Cl (5.09 g, 95 mmol, 10 equiv). The mixture was stirred at 80° C. for 12 h. The reaction mixture was filtered through celite, and the filtrate was concentrated in vacuum to give a residue. The residue was diluted with water (300 ml) and extracted with EtOAc (80 ml×3). The combined organic layers were dried over anhydrous Na2SO4, filtered and the filtrate was concentrated in vacuum. The residue was purified by silica gel column chromatography eluted with EtOAc/MeOH (10:1) to afford 404-6 (1.16 g, 35.5%) as a white solid.

1H-NMR-404-6: (400 MHz, DMSO-d6) δ 8.34 (s, 1H), 6.95 (t, J=8.0 Hz, 1H), 6.48-6.27 (m, 3H), 5.10 (s, 2H), 4.29 (d, J=10.8 Hz, 1H), 3.95 (d, J=4.0 Hz, 1H), 3.71 (s, 1H), 3.65-3.58 (m, 1H), 3.48 (s, 1H), 3.33 (s, 4H), 1.36 (s, 9H)

### 7. Synthesis of 404-7

To a solution of 404-6 (1.16 g, 3.38 mmol, 1 equiv) and 1-2 (967 mg, 3.38 mmol, 0.9 equiv) in MeOH (20 mL) was added AcOH (579 uL, 10.1 mmol, 3 equiv), the mixture was stirred at 25° C. for 1 h under nitrogen atmosphere, then NaBH3CN (424 mg 6.76 mmol, 2 equiv) was added to the mixture, the result mixture was stirred at 25° C. for 1 h under nitrogen atmosphere. The reaction mixture was concentrated in vacuum to remove MeOH, the residue was diluted with Sat. NaHCO₃ (200 mL), extracted with EtOAc (50 mL×3). The combined organic layers were dried over anhydrous Na2SO4, filtered and the filtrate was concentrated in vacuum. The residue was purified by silica gel column chromatography eluted with DCM/MeOH (10:1) to afford 404-7 (1.8 g, 86% yield) as a gray solid.

1H-NMR-404-7: (400 MHz, DMSO-d6) δ 8.69 (s, 1H), 8.36-8.27 (m, 1H), 8.03 (s, 1H), 7.01 (t, J=7.8 Hz, 1H), 6.60-6.47 (m, 2H), 6.38 (d, J=7.6 Hz, 1H), 6.21 (t, J=5.6 Hz, 1H), 4.44 (d, J=4.8 Hz, 2H), 4.31 (d, J=10.8 Hz, 1H), 3.95 (s, 1H), 3.70-3.41 (m, 6H), 3.28 (s, 3H), 2.68 (s, 2H), 1.95-1.84 (m, 1H), 1.67-1.55 (m, 4H), 1.55-1.39 (m, 2H), 1.36 (s, 9H), 0.81 (d, J=6.0 Hz, 3H)

### 8. Synthesis of 404-8

To a solution of 404-7 (0.9 g, 147 mmol, 1 equiv) in DCM (7 ml) was added pyridine (710 uL, 8.8 mmol, 6 equiv) and bis(trichloromethyl) carbonate (630 mg, 2.12 mmol, 1.45 equiv) at 0° C. The mixture was stirred at 25° C. for 1.5 h under nitrogen atmosphere. The reaction mixture was diluted with Sat. NaHCO₃ (100 mL), extracted with CH2Cl2 (50 mL×3). The combined organic layers were dried over anhydrous Na2SO4, filtered and the filtrate was concentrated in vacuum. The residue was purified by silica gel column chromatography eluted with DCM/MeOH (10:1) to afford 404-8 (926 mg, 98%) as a yellow solid.

1H-NMR-404-8: (400 MHz, DMSO-d6) δ 8.37 (s, 1H), 7.81-7.72 (m, 2H), 7.65 (s, 1H), 7.48 (t, J=8.0 Hz, 1H), 7.33 (d, J=2.4 Hz, 1H), 7.20 (d, J=7.6 Hz, 1H), 7.01 (s, 1H), 4.62 (d, J=10.8 Hz, 1H), 4.57 (s, 1H), 4.05-3.92 (m, 1H), 3.71 (d, J=5.6 Hz, 2H), 3.57-3.48 (m, 2H), 3.39 (s, 3H), 3.25 (s, 2H), 2.81-2.69 (m, 2H), 1.96-1.84 (m, 1H), 1.67-1.56 (m, 4H), 1.45 (d, J=12.4 Hz, 1H), 1.36 (s, 9H), 0.82 (d, J=6.0 Hz, 3H)

### 9. Synthesis of 404-9&10

The 404-8 (0.2 g) was purified by Chiral separation with the following conditions (column: REGIS(S,S)WHELK-O1 (250 mm*25 mm, 10 um); mobile phase: [Neu-ETOH]; B %: 50%-50%, 20 min, Flow rate: 70 mL/min; Wave Length: 220/254 nm; RT1 (min): 4.71; RT2 (min): 6.03) to afford 404-9 (50.3 mg, 89%) as a white solid and 404-10 (52.9 mg, 95%) as a white solid.

1H-NMR-404-9: (400 MHz, DMSO-d6) δ 8.37 (s, 1H), 7.81-7.73 (m, 2H), 7.65 (s, 1H), 7.48 (t, J=8.0 Hz, 1H), 7.37 (s, 1H), 7.20 (d, J=7.6 Hz, 1H), 7.01 (s, 1H), 4.62 (d, J=10.8 Hz, 1H), 4.07-3.94 (m, 1H), 3.77-3.64 (m, 2H), 3.57-3.46 (m, 2H), 3.38 (s, 2H), 3.24 (s, 3H), 2.80-2.70 (m, 2H), 1.89 (t, J=10.4 Hz, 1H), 1.68-1.56 (m, 4H), 1.45 (d, J=12.4 Hz, 1H), 1.36 (s, 9H), 1.23 (s, 1H), 0.83 (d, J=6.0 Hz, 3H)

1H-NMR-404-10: (400 MHz, DMSO-d6) δ 8.37 (s, 1H), 7.82-7.72 (m, 2H), 7.67 (s, 1H), 7.48 (t, J=8.0 Hz, 1H), 7.36 (s, 1H), 7.20 (d, J=7.6 Hz, 1H), 7.03 (s, 1H), 4.62 (d, J=10.8 Hz, 1H), 3.99 (d, J=1.2 Hz, 1H), 3.70 (d, J=5.5 Hz, 2H), 3.52 (s, 5H), 2.78 (s, 2H), 2.03-1.86 (m, 1H), 1.71-1.55 (m, 4H), 1.51-1.44 (m, 1H), 1.36 (s, 9H), 1.23 (s, 1H), 0.83 (d, J=6.0 Hz, 3H)

### 10. Synthesis of 404

To a solution of 404-9 (50.3 mg, 78.6 umol, 1 equiv) in DCM (1 mL) was added HCl/dioxane (78.6 ul, 4 M, 4 equiv). The mixture was stirred at 25° C. for 1 h. The mixture was concentrated under reduced pressure. The residue was purified by Prep-HPLC with the following conditions (column: Phenomenex Luna C18 75*30 mm*3 um; Mobile Phase A: water (NH4HCO3)), Mobile Phase B: ACN; Flow rate: 25 mL/min; Gradient: 1% B to 25% B in 8 min; Wave Length: 220 nm; RT1 (min): 8.2) to afford 404 (9.6 ng, 22%) as a yellow solid.

MS-404: (ES, m/z): [M+H]+ 540.2. 1H-NMR-404: (400 MHz, METHANOL-d4) δ 8.54 (s, 1H), 8.47-8.39 (m, 1H), 7.81-7.63 (m, 3H), 7.57 (t, J=8.0 Hz, 1H), 7.26 (d, J=7.5 Hz, 1H), 7.16 (d, J=16.4 Hz, 2H), 4.75 (d, J=10.0 Hz, 1H), 4.28 (s, 1H), 4.14-3.87 (m, 4H), 3.56 (s, 2H), 3.52-3.44 (m, 3H), 3.13-2.96 (m, 2H), 2.23 (t, J=10.8 Hz, 1H), 1.93 (t, J=10.8 Hz, 1H), 1.85-1.65 (m, 4H), 1.07-0.96 (m, 1H), 0.92 (d, J=6.0 Hz, 3H).

### Example 400. Synthesis of Compound 404_P2

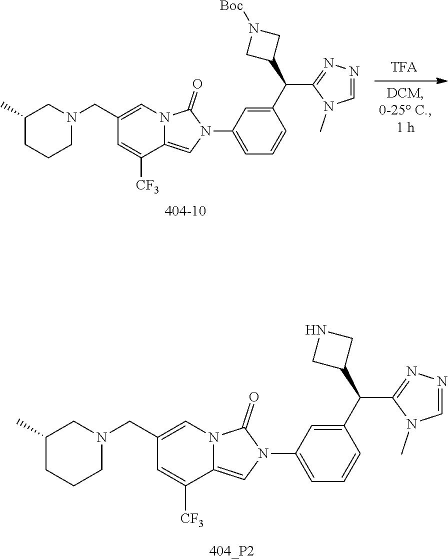 [SMILES: "TFA" ; C[C@H]1CCCN(Cc2cc(C(F)(F)F)c3cn(-c4cccc([C@H](c5nncn5C)C5CN(C(=O)OC(C)(C)C)C5)c4)c(=O)n3c2)C1 ; C[C@H]1CCCN(Cc2cc(C(F)(F)F)c3cn(-c4cccc([C@H](c5nncn5C)C5CNC5)c4)c(=O)n3c2)C1]

### 1. Synthesis of 404

To a solution of 404-10 (42.9 mg, 67 umol, 1 equiv) in DCM (1 ml) was added HCl/dioxane (67 ul, 4 M, 4 equiv). The mixture was stirred at 25° C. for 1 h. The mixture was concentrated under reduced pressure. The residue was purified by Prep-HPLC with the following conditions (column: Phenomenex Luna C18 75*30 mm*3 um; Mobile Phase A: water (NH4HCO3)), Mobile Phase B: ACN; Flow rate: 25 mL/min; Gradient: 1% B to 25% B in 8 min; Wave Length: 220 nm; RT1 (min): 8.4) to afford 403 (6 mg, 15%) as a yellow solid.

MS-404: (ES, m/z): [M+H]⁺ 540.1. ¹H-NMR-404: (400 MHz, METHANOL-d4) δ 8.51 (s, 1H), 8.46-8.39 (m, 1H), 7.86-7.64 (m, 3H), 7.57 (t, J=8.0 Hz, 1H), 7.24 (d, J=7.6 Hz, 1H), 7.15 (d, J=16.8 Hz, 2H), 4.72 (d, J=9.2 Hz, 1H), 4.27 (t, J=8.0 Hz, 1H), 4.16-3.85 (m, 4H), 3.55-3.49 (m, 2H), 3.46 (s, 3H), 3.11-2.90 (m, 2H), 2.27-2.11 (m, 1H), 1.89 (t, J=10.0 Hz, 1H), 1.82-1.62 (m, 4H), 0.99 (d, J=9.2 Hz, 1H), 0.92 (d, J=6.0 Hz, 3H).

### Example 401. Synthesis of Compound 404 and 405

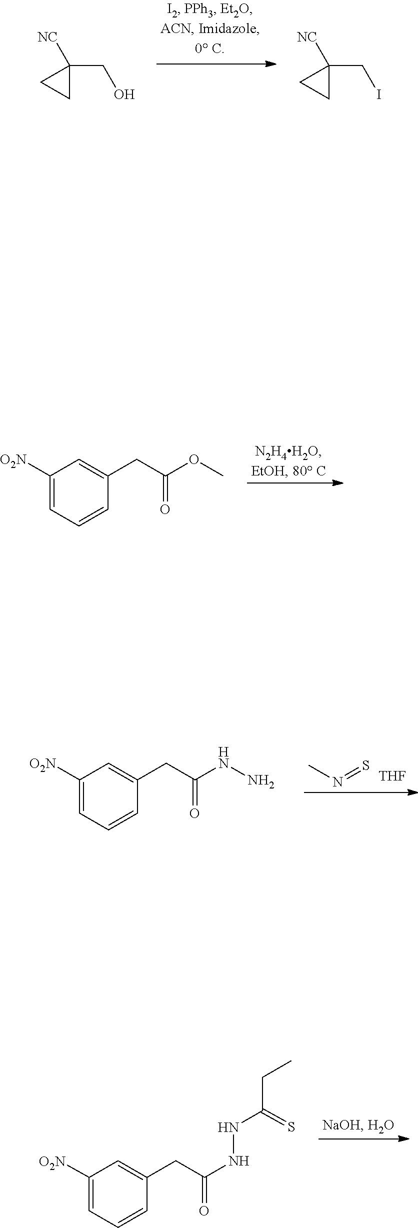

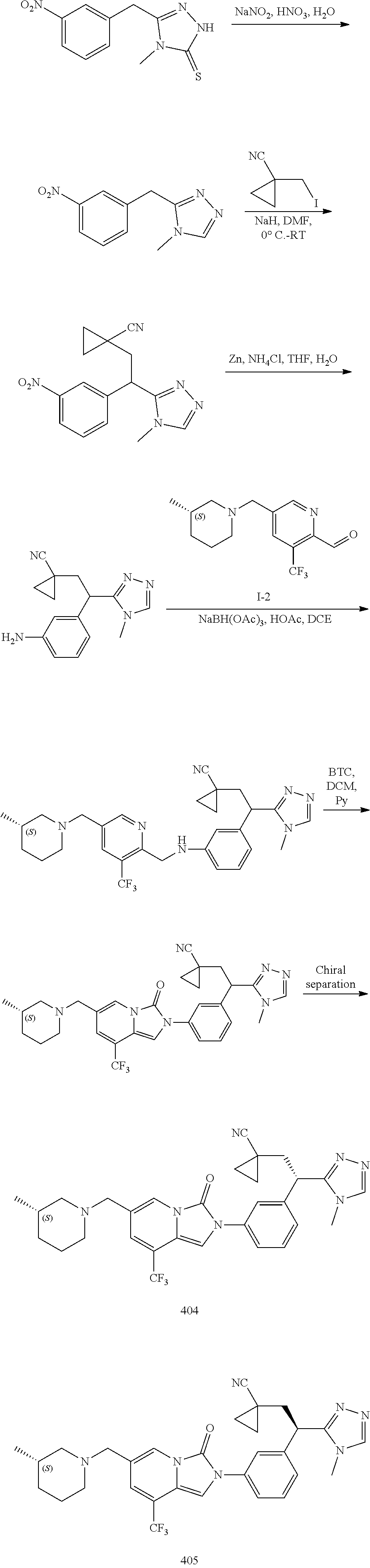

### 1. Synthesis of 404-1

Into a 500 mL 3-necked round-bottom flask were added 1-(hydr-oxymethyl)cyclopropane-1-carbonitrile (10 g, 1012.969 mmol, 1 equiv), Et₂O (150 mL), ACN (75 mL), imidazole (9.81 g, 144,157 mmol, 1.4 equiv), 12 (39.20 g, 154.453 mmol, 1.5 equiv) and PPh₃ (35.11 g, 133,860 mol, 1.3 equiv) at 0° C. The resulting mixture was stirred for overnight at room temperature under nitrogen atmosphere. The reaction was quenched by the addition of Na2SO3 (500 mL) at room temperature. The aqueous layer was extracted with EtOAc (3×300 mL). The resulting mixture was concentrated under reduced pressure. The residue was purified by silica gel column chromatography, eluted with PE/EA (20:1) to afford 404-1 (16 g, 75.06%) as a yellow oil.

### 2. Synthesis of 404-2

Into a 500 mL 3-necked round-bottom flask were added methyl 2-(3-nitrophenyl)acetate (30 g, 153.709 mmol, 1 equiv) and hydrazine hydrate (98%) (77.8 g 1554.104 mmol, 10.11 equiv) in EtOH (300 mL) at room temperature. The resulting mixture was stirred for overnight at 80° C. under nitrogen atmosphere. The reaction was quenched with oater (200 mL) at room temperature. The aqueous layer was extracted with EA (3×300 mL). The resulting mixture was concentrated under reduced pressure. This resulted in 404-2 (28 g, 83.06%) as a white solid.

### 3. Synthesis of 404-3

Into a 1000 mL 3-necked round-bottom flask were added methyl isothiocyanate (25.59 g, 350,040 mmol, 2.44 equiv), tetrahydrofuran (280 mL) and 404-2 (28 g, 143.459 mmol, 1 equiv) at room temperature. The resulting mixture was stirred for overnight at room temperature. The reaction was quenched with Water (200 mL) at room temperature. The precipitated solids were collected by filtration and washed with water (3×20 mL). This resulted in 404-3 (35 g, 90.96%) as a yellow solid.

### 4. Synthesis of 404-4

Into a 500 mL 3-necked round-bottom flask were added 1 M NaOH (1300 mL, aq) and 404-3 (35 g, 130.46 mmol, 1 equiv) at room temperature. The resulting mixture was stirred for overnight at room temperature. The mixture was acidified to pH 5 with 1M HCl (1300 mL). The precipitated solids were collected by filtration and washed with water (3×10 mL). This resulted in 404-4 (30 g, 91.86%) as a yellow solid.

### 5. Synthesis of 404-5

To a stirred solution of 404-4 (30 g, 120 mmol, 1 equiv) and NaNO₂ (82.8 g, 1200 mmol, 10 equiv) in water (250 mL) was added HNO3 (75.6 g, 1200 mmol, 10 equiv) dropwise at 0° C. The resulting mixture was stirred for overnight at room temperature. The mixture neutralized to pH 7 with sat. NaHCO₃ (1000 mL). The aqueous layer was extracted with EA (3×500 mL). The residue was purified by silica gel column chromatography, eluted with PE./EA (10:1) to afford 404-5 (25.8 g, 98.72%) as a yellow solid.

### 6. Synthesis of 404-6

Into a 250 mL 3-necked round bottom flask were added 404-5 (5 g, 22.913 mmol, 1 equiv), DMF (60 mL) and NaH (0.66 g, 27.496 mmol, 1.2 equiv) at 0° C. The resulting mixture was stirred for 1 h at 0° C. To the above mixture was added 404-1 (4.74 g, 22.913 mmol, 1 equiv) at 0° C. The resulting mixture was stirred for additional 3 h at room temperature. The reaction was quenched by the addition of sat. NH4Cl (300 mL) at room temperature. The resulting mixture was extracted with EtOAc (3×200 ml). The resulting mixture was concentrated under reduced pressure. The residue was purified by silica gel column chromatography, eluted with DCM/MeOH (100:1) to afford 404-6 (420 mg, 6.18%) as a light yellow solid.

### 7. Synthesis of 404-7

Into a 30 mL sealed tube were added 404-6 (420 mg, 1.413 mmol, 1 equiv), TI-IF (8 mL), NH₄Cl (377.91 mg, 7.065 mmol, 5 equiv), H₂O (2 mL) and Zn (1385.73 mg, 21.195 mmol, 15 equiv) at room temperature. The resulting mixture was stirred for 1 h at room temperature. The resulting mixture was filtered; the filter cake was washed with THF (3×5 mL). The filtrate was concentrated under reduced pressure. The residue was purified by Prep-TLC (DCM/MeOH=15:1) to afford 404-7 (300 mg, 79.45%) as a light yellow solid.

### 8. Synthesis of 404-8

Into a 50 mL round bottom flask were added 404-7 (300 mg, 1.122 mmol, 1 equiv), DCE (3 mL), I-2 (321.28 mg, 1.122 mmol, 1 equiv) and NaBH(OAc)3 (475.67 mg, 2.244 mmol, 2 equiv) at room temperature. The resulting mixture was stirred for 2 h at room temperature. The reaction was quenched with water at room temperature. The aqueous layer was extracted with DCM/MeOH=10:1 (3×10 mL). The resulting mixture was concentrated under reduced pressure. The residue was purified by Prep-TLC (DCM/MeOH=15:1) to afford 404-8 (300 mg, 49.72%) as a yellow oil.

### 9. Synthesis of 404-9

Into a 100 ml, round-bottom flask were added 404-8 (300 mg, 0,558 mmol, 1 equiv), DCM (12 mL) and pyridine (264.83 mg, 3.348 mmol, 6 equiv) and triphosgene (66.23 mg, 0.223 mmol, 0.4 equiv) at 0° C. The resulting mixture was stirred for 5 min at 0° C. The reaction was quenched by the addition of sat. NaHCO₃ (50 mL) at room temperature. The aqueous layer was extracted with DCM (3×20 mL). The residue was purified by Prep-TLC (DCM/MeOH 15:1) to afford 404-9 (190 mg, 60.41%) as a yellow solid.

### 10. Synthesis of 404-0

The 404-9 (190 mg, 0.320 mmol, 1 equiv, 95%) was purified by Chiral separation with the following conditions (Column: Chiralpak IG, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 40% B to 40% B in 8.5 min; Wave Length: 220/254 nm; RT1 (min): 5.09; RT2 (min): 6.74; The first peak was the 404; the second peak was 405. Sample Solvent: EtOH:DCM=1:1-HPLC; Injection Volume: 0.8 mL; Number of Runs: 6) to afford 404 (46.6 mg, 24.53%) as a yellow solid and 405 (55.9 mg, 29.42%) as a yellow solid.

LC-MS-404 (ES, m/z): [M+H]⁺ 564

H-NMR-404 1H NMR (300 MHz, DMSO-d6 ppm) δ 0.65-0.70 (m, 1H), 0.96-1.00 (m, 4H), 1.09-1.11 (m, 2H), 1.20-1.23 (m, 1H), 1.32-1.48 (m, 1H), 1.58-12.63 (m, 4H), 1.88-1.92 (m, 1H), 2.24-2.30 (m, 1H), 2.50-2.57 (m, 1H), 2.73-2.75 (m, 2H), 3.25-3.33 (m, 2H), 3.51 (s, 3H), 4.48-4.52 (m, 1H), 7.01 (s, 1H), 7.30-7.33 (m, 2H), 7.47-7.51 (m, 1H), 7.66 (s, 1H), 7.77-7.80 (m, 2H), 8.39 (s, 1H).

LC-MS-405 (ES, m/z): [M+H]⁺ 564

H-NMR-405 1H NMR (300 MHz, DMSO-d6 ppm) δ 0.65-0.70 (m, 1H), 0.99-1.02 (m, 4H), 1.09-1.11 (m, 2H), 1.32-1.48 (m, 1H), 1.58-1.65 (m, 1H), 1.85-1.90 (m, 4H), 1.90-2.08 (m, 1H), 2.24-2.30 (m, 1H), 2.50-2.57 (m, 1H), 2.73-2.75 (m, 2H), 3.25-3.44 (m, 2H), 3.51 (s, 3H), 4.48-4.52 (m, 1H), 7.01 (s, 1H), 7.30-7.33 (m, 2H), 7.47-7.51 (m, 1H), 7.66 (s, 1H), 7.77-7.80 (m, 2H), 8.39 (s, 1H).

### Example 402. Synthesis of Compound 406 and 407

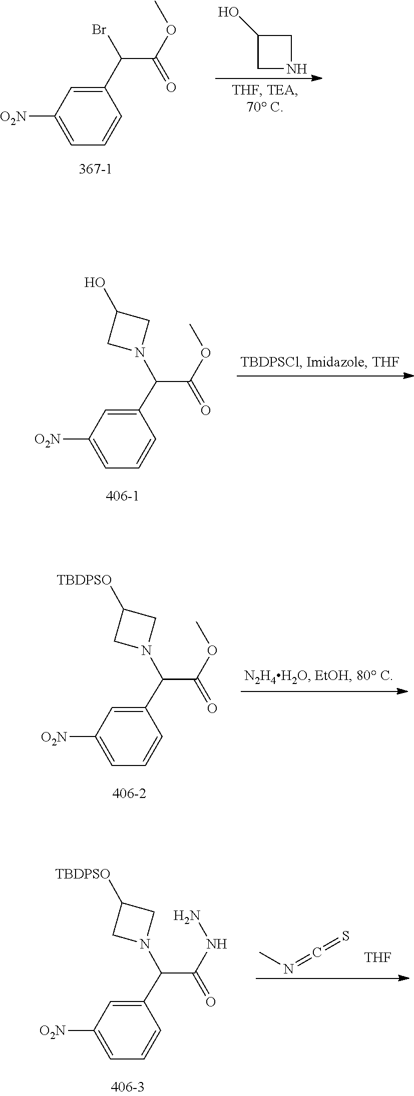

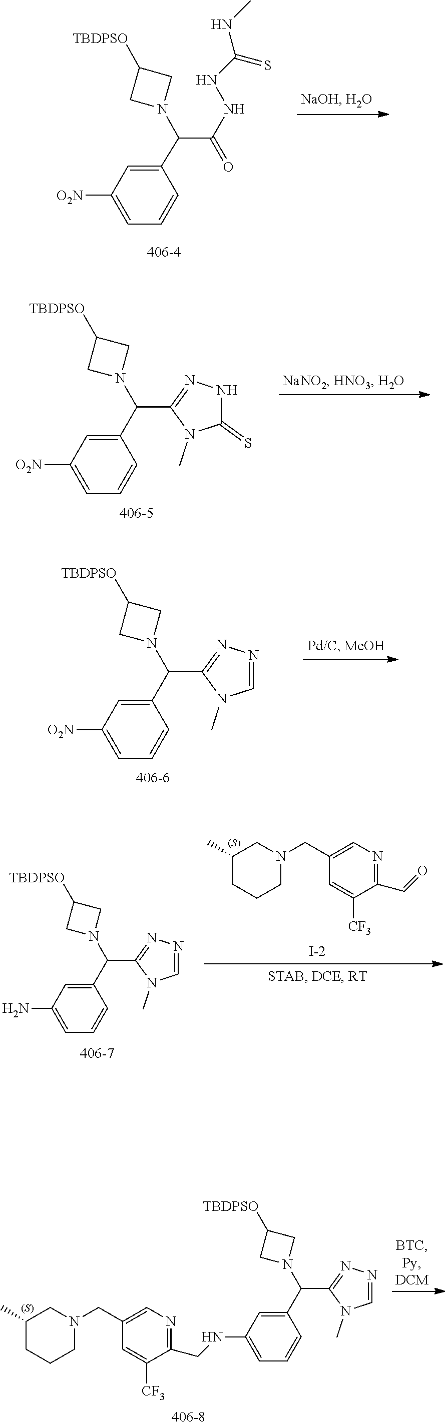

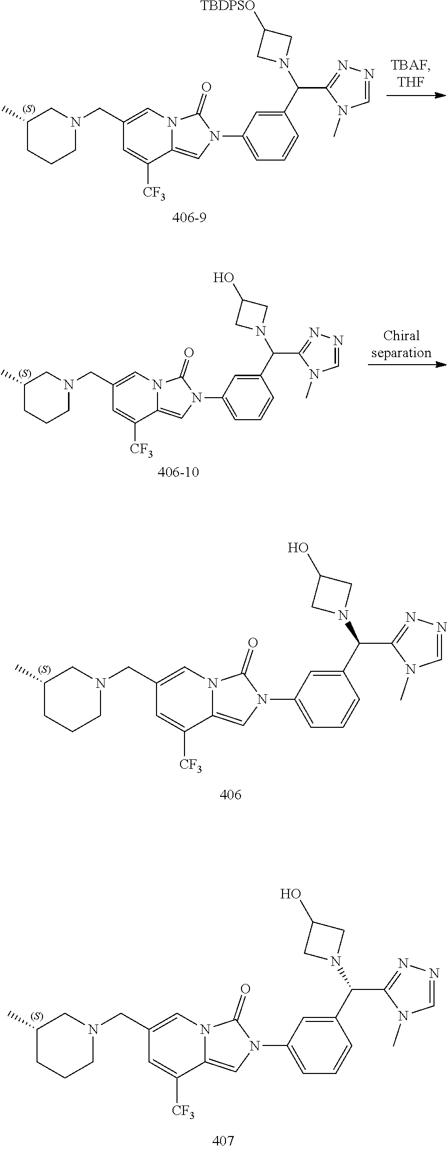

### 1. Synthesis of 406-1

To a stirred solution of 3671 (5 g, 18,244 mmol, 1 equiv), THF (50 mL), and azetidin-3-ol (1.60 g, 21.893 mmol, 1.2 equiv) was added TEA (3.69 g, 36,488 mmol, 2 equiv). The resulting mixture was stirred for overnight at 70° C. The mixture was allowed to cool down to room temperature. The reaction was quenched by the addition of water (100 mL) at room temperature. The aqueous layer was extracted with EtOAc (1×100 mL). The resulting mixture was concentrated under vacuum. The residue was purified by silica gel column chromatography, eluted with PE/EA (7:1) to afford 406-1 (3.4 g, 70.00%) as a yellow oil.

### 2. Synthesis of 406-2

To a stirred solution of 406-1 (34 g, 12.770 mmol, 1 equiv), THF (10 mL) and Imidazole (2.61 g, 38.310 mmol, 3 equiv) was added TBDPSCl (14.04 g, 51.080 mmol, 4 equiv). The resulting mixture was stirred for overnight at room temperature. The reaction was quenched by the addition of water (20 mL) at room temperature. The aqueous layer was extracted with EtOAc (1×10 mL). The resulting mixture was concentrated under vacuum. The residue was purified by silica gel column chromatography, eluted with PE/EA (8:1) to afford 406-2 (4.2 g, 65.17%) as a yellow oil.

### 3. Synthesis of 406-3

To a stirred solution of 406-2 (4 g, 7.926 mmol, 1 equiv) and EtOH (40 mL) was added NH₂NH₂·H₂O (3.17 g, 63.408 mmol, 8 equiv). The resulting mixture was stirred for overnight at 80° C. The mixture was allowed to cool down to room temperature. The reaction was quenched by the addition of water (100 mL) at room temperature. The aqueous layer was extracted with EtOAc (1×100 mL). The resulting mixture was concentrated under vacuum. The residue was purified by silica gel column chromatography, eluted with CH₂Cl₂/MeOH (10:1) to afford 406-3 (3.6 g, 90.00%) as a yellow oil.

### 4. Synthesis of 406-4

To a stirred solution of 406-3 (3.6 g, 7.133 mmol, 1 equiv) and tetrahydrofuran (50 mL) was added methyl isothiocyanate (1.30 g, 17.832 mmol, 2.5 equiv). The resulting mixture was stirred for overnight at room temperature. The resulting mixture was concentrated under vacuum and wash with water (50 mL). The precipitated solids were collected by filtration and washed with water (1×100 mL). The resulting solid was dried under vacuum. This resulted in 406-4 (3.7 g, 89.77%) as a yellow solid.

### 5. Synthesis of 406-5

Into a 100 mL 3-necked round-bottom flask were added 406-4 (3.7 g, 6.404 mmol, 1 equiv) and NaOH (aq. 1 mol/L) (1.02 g, 25.616 mmol, 4 equiv). The resulting mixture was stirred for overnight at room temperature. The mixture was acidified to pH 4 with conc. HCl (1 M, 44 mL). The aqueous layer was extracted with EtOAc (1×10 mL). The resulting mixture was concentrated under vacuum. The residue was purified by silica gel column chromatography, eluted with PB/EA (1:1) to afford 406-5 (3.3 g, 92.06%) as a yellow solid.

### 6. Synthesis of 406-6

To a stirred mixture of 406-5 (3.3 g, 5.895 mmol, 1 equiv) and NaNO₂ (aq. 1 mol/L, 4.07 g, 58.950 mmol, 10 equiv) in EtOAc (60 mL) was added HNO₃ (aq. 1 mol/L, 3.71 g, 58.832 mmol, 10 equiv) dropwise at room temperature. The resulting mixture was stirred for overnight at room temperature. The mixture was acidified to pH 8 with saturated NaHCO₃ (aq. 40 mL). The aqueous layer was extracted with DCM (3×20 mL). The resulting mixture was concentrated under vacuum. The residue was purified by silica gel column chromatography, eluted with CH₂Cl₂/MeOH (10:1) to afford 406-6 (26 g, 83.57%) as a yellow solid.

### 7. Synthesis of 406-7

To a solution of 406-6 (2.6 g, 4.927 mmol, 1 equiv) in 30 mL MeOH was added Pd/C (10%, 0.52 g) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. The crude product 406-7 (2.4 g, 97.87%) as a yellow solid was used in the next step directly without further purification.

### 8. Synthesis of 406-8

To a stirred solution of 406-7 (2.2 g, 4.420 mmol, 1 equiv) and DCE (25 mL) was added I-2 (1.52 g, 5.304 mmol, 1.2 equiv) room temperature. The resulting mixture was stirred for overnight at room temperature. To the above mixture was added STAB (1.87 g, 8.840 mmol, 2 equiv) at room temperature. The resulting mixture was stirred for additional 4 h at room temperature. The reaction was quenched by the addition of sat. NaHCO₃ (aq. 30 mL) at room temperature. The aqueous layer was extracted with CH₂Cl₂ (1×30 mL). The resulting mixture was concentrated under vacuum. The residue was purified by silica gel column chromatography, eluted with CH₂Cl₂/MeOH (10:1) to afford 406-8 (1.7 g, 50.08%) as a yellow oil.

### 9. Synthesis of 406-9

To a stirred solution of 406-8 (1.6 g, 2.083 mmol, 1 equiv), DCM (20 mL) and pyridine (1.65 g, 20.830 mmol, 10 equiv) was added triphosgene (0.22 g, 0,729 mmol, 0.35 equiv) at 0° C. The resulting mixture was stirred for 15 min at room temperature. The reaction was quenched by the addition of sat. NaHCO₃ (aq.) (30 mL) at room temperature. The aqueous layer was extracted with CH₂Cl₂/MeOH (10/1) (2×10 mL). The resulting mixture was concentrated under vacuum. The residue was purified by silica gel column chromatography, eluted with CH₂Cl₂/MeOH (12:1) to afford 406-9 (410 mg, 24.79%) as a yellow solid.

### 10. Synthesis of 406-10

To a stirred solution of 406-9 (400 mg, 0.504 mmol, 1 equiv) and THF (5 mL) was added TBAF (197.58 mg, 0.756 mmol, 1.5 equiv). The resulting mixture was stirred for overnight at room temperature. The reaction was quenched by the addition of water (5 mL) at room temperature. The aqueous layer was extracted with CH₂Cl₂ (3×10 mL). The resulting mixture was concentrated under vacuum. The crude product was purified by reverse flash chromatography with the following conditions: column, C18; mobile phase, A: water (10 mmol/L NH4HCO₃), B: CH3CN, 20% B to 80% B gradient in 20 min; detector, UV 254 nm. This resulted in product. The residue was dried by lyophilization to afford 406-10 (120 mg, 42.87%) as a yellow oil.

### 11. Synthesis of 406 and 407

The 406-10 (120 mg) was purified by Chiral separation with the following conditions (Column: CHIRALPAK IG, 2*25 cm, 5 Lim; Mobile Phase A: Hex (0.5% 2M NH₃-MeOH)-HPLC, Mobile Phase B: IPA: DCM=1:1-HPLC; Flow rate: 20 mL/min; Gradient: 30% B to 30% B in 15 min; Wave Length: 220/254 am; RT1 (min): 8.82; RT2 (min): 11.41; The first peak is 407; the second peak is 406; Sample Solvent: IPA: DCM=1:1-HPLC; Injection Volume: 0.6 mL; Number of Runs: 5) to afford crude product. The crude product (21 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 3% B to 15% B in 7 min; Wave Length: 254; 220 nm; RT1 (min): 5.65) to afford 407 (13.0 mg, 9.95%) as a yellow solid and 406 (11.6 mg, 8.89%) as a yellow solid.

LC-MS-407 (ES, m/z): [M+H]⁺ 556. H-NMR-407 1H NMR (400 MHz, DMSO-d6 ppm) δ 0.82-0.89 (m, 4H), δ 1.41-1.50 (m, 1H), δ 1.58-1.63 (m, 4H), δ 1.87-1.92 (t, 1H), δ 2.67-2.78 (m, 2H), δ 2.80-2.87 (m, 1H), δ 2.87-2.89 (t, 1H), δ 3.25 (s, 2H), δ 3.38 (s, 1H), δ 3.57 (s, 4H), δ 4.25-4.28 (t, 1H), δ 4.97 (s, 1H), δ 5.40 (s, 1H), δ 7.01 (s, 1H), δ 7.33-7.36 (m, 2H), δ 7.46-7.50 (t, 1H), δ 7.66 (s, 2H), δ 7.95 (s, 1H), δ 8.21 (s, 1H), δ 8.32 (s, 1H).

LC-MS-406 (ES, m/z): [M+H]⁺ 556. H-NMR-406 1H NMR (400 MHz, DMSO-d6 ppm) δ 0.82-0.86 (m, 4H), δ 1.41-1.50 (m, 1H), δ 1.58-1.66 (m, 4H), δ 1.87-1.92 (t, 1H), δ 2.73-2.80 (m, 2H), δ 2.81-2.83 (t, 1H), δ 2.86-2.89 (t, 1H), δ 3.22 (s, 2H), δ 3.38-3.42 (m, 1H), δ 3.52-3.77 (m, 4H), δ 4.24-4.28 (t, 1H), δ 4.97 (s, 1H), δ 7.01 (s, 1H), δ 7.32-7.36 (m, 2H), δ 7.46-7.50 (t, 1H), δ 7.67 (s, 2H), δ 7.95 (s, 1H), δ 8.20 (s, 1H), δ 8.33 (s, 1H).

### Example 403. Synthesis of Compound 408 and 409

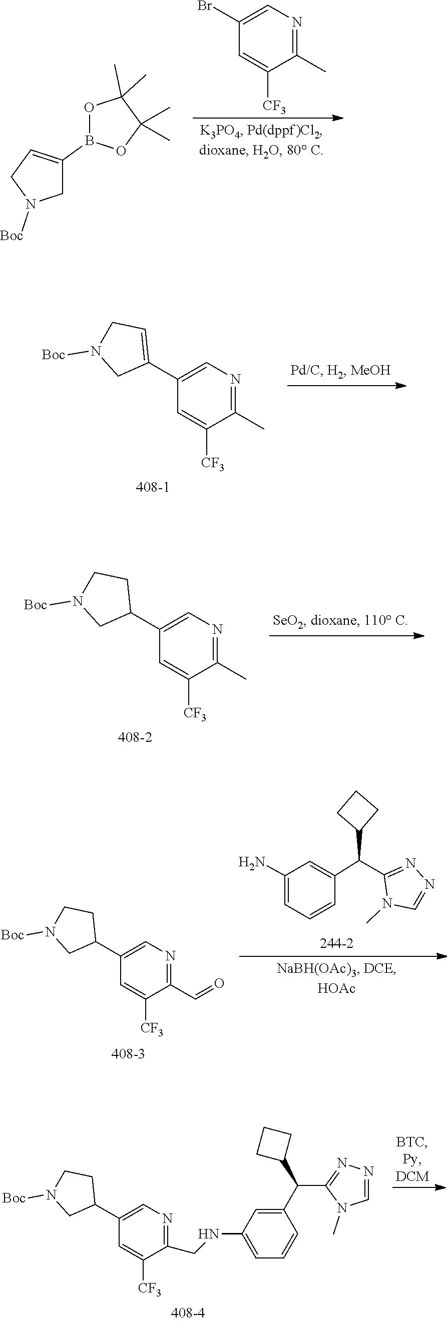

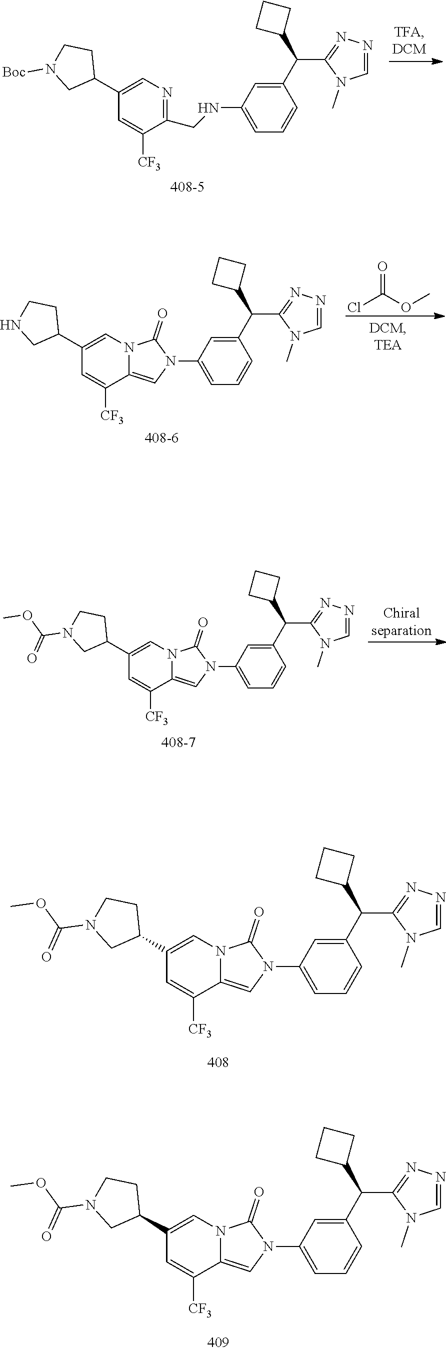

### 1. Synthesis of 408-1

Into a 250 mL 3-necked round-bottom flask were added tert-butyl 3-(4,4,5,5-tetramethyl-1,3,2-dioxaborolan-2-yl)-2,5-dihydropyrrole-1-carboxylate (5 g, 16.938 mmol, 1 equiv), dioxane (50 mL), H2O (12.5 mL), 5-bromo-2-methyl-3-(trifluoromethyl)pyridine (4.07 g, 16.938 mmol, 1 equiv), K₃PO₄ (10.79 g, 50.814 mol, 3 equiv) and Pd(dppf)Cl2 (1.24 g, 1.694 mmol, 0.1 equiv) at room temperature. The resulting mixture was stirred for overnight at 80° C. under nitrogen atmosphere. The reaction was quenched by the addition of water (100 mL) at room temperature. The aqueous layer was extracted with EtOAc (3×80 mL). The resulting mixture was concentrated under reduced pressure. The residue was purified by silica gel column chromatography, eluted with PE/EA (10:1) to afford 408-1 (5 g, 89.91%) as a grey solid.

### 2. Synthesis of 408-2

To a solution of 408-1 (5 g, 15,228 mmol, 1 equiv) in 60 mL MeOH was added Pd/C (10%, 1.62 g) in a pressure tank. The mixture was hydrogenated at room temperature under 30 psi of hydrogen pressure for 2 h, filtered through a Celite pad and concentrated under reduced pressure to afford 408-2 (4.2 g, 83.49%) as a light brown solid.

### 3. Synthesis of 408-3

Into a 250 mL 3-necked round-bottom flask were added 408-2 (4 g, 12.108 mmol, 1 equiv), dioxane (50 ml) and SeO₂ (6.72 g, 60.540 mmol, 5 equiv) at room temperature. The resulting mixture was stirred for overnight at 100° C. under nitrogen atmosphere. The resulting mixture was filtered; the filter cake was washed with MeOH (3×10 ml). The filtrate was concentrated under reduced pressure. The residue was purified by silica gel column chromatography, eluted with PE/EA (10:1) to afford 408-3 (1.1 g, 26.38%) as a light yellow solid.

### 4. Synthesis of 408-4

Into a 100 mL 3-necked round-bottom flask were added 408-3 (1 g, 2.904 mmol, 1 equiv), DCE (15 ml), 244b (0.70 g, 2,904 mmol, 1 equiv), HOAc (0.17 g, 2,904 mmol, 1 equiv) and NaBH(OAc)₃ (1.23 g, 5.808 mmol, 2 equiv) at room temperature. The reaction was quenched by the addition of sat, NH₄Cl (aq.) (20 mL) at room temperature. The aqueous layer was extracted with EtOAc (3×10 mL). The resulting mixture was concentrated under reduced pressure. The residue was purified by silica gel column chromatography, eluted with CH₂Cl₂/MeOH (100:1) to afford 408-4 (1 g, 60.34%) as a light yellow solid.

### 5. Synthesis of 408-5

Into a 100 mL 3-necked round-bottom flask were added 408-4 (1 g, 1.752 mmol, 1 equiv), DCM (15 mL), pyridine (0.83 g, 10.512 mmol, 6 equiv) and triphosgene (0.21 g, 0,701 mmol, 0.4 equiv) at 0° C. The resulting mixture was stirred for 10 min at 0° C. under nitrogen atmosphere. The reaction was quenched by the addition of sat. NaHCO₃ (aq.) (15 mL) at room temperature. The aqueous layer was extracted with CH₂Cl₂ (3×10 mL). The resulting mixture was concentrated under reduced pressure. The residue was purified by silica gel column chromatography, eluted with CH₂Cl₂/MeOH (80:1) to afford 408-5 (800 mg, 76.51%) as a light yellow solid.

### 6. Synthesis of 408-6

Into a 100 mL round-bottom flask were added 408-5 (780 mg, 1.307 mmol, 1 equiv), DCM (9 mL) and TFA (3 mL) at room temperature. The resulting mixture was stirred for 1 h at room temperature. The resulting mixture was concentrated under reduced pressure. The residue was purified by silica gel column chromatography, eluted with CH₂Cl₂/MeOH (10:1) to afford 408-6 (500 mg, 77.03%) as a light yellow solid.

### 7. Synthesis of 408-7

Into a 100 mL, round-bottom flask were added 408-6 (230 mg, 0,463 mmol, 1 equiv), DCM (5 ml), TEA (140.62 mg, 1.389 mmol, 3 equiv) and methyl chloroformate (43.77 mg, 0,463 mmol, 1 equiv) at room temperature. The resulting mixture was stirred for 2 h at room temperature. The reaction was quenched by the addition of water (15 mL) at room temperature. The aqueous layer was extracted with CH₂Cl₂ (3×10 mL). The resulting mixture was concentrated under reduced pressure. The residue was purified by Prep-TLC eluted with CH₂Cl₂/MeOH (10:1) to afford 408-7 (250 mg, 97.32%) as a light yellow solid.

### 8. Synthesis of 408 and 409

The 408-7 (250 mg) was purified by Chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 90% B to 90% B in 13 min; Wave Length: 220/254 um; RT1 (min): 8.97; RT2 (min): 10.97; The first peak was the 408; the second peak was 409. Sample Solvent: EtOH:DCM=1:1-HPLC; Injection Volume: 0.3 mL; Number of Runs: 15) to afford 408 (101.4 mg, 40.56%) as a light yellow solid and 409 (94.5 mg, 37.80%) as a light yellow solid.

LC-MS-408 (ES, m/z): [M+H]⁺ 555. H-NMR-408 1H NMR (300 MHz, DMSO-d6 ppm) δ 1.60-1.89 (m, 5H), 1.90-2.09 (m, 1H), 2.06-2.13 (m, 2H), 3.18-3.33 (m, 4H), 3.43-3.48 (m, 3H), 3.50 (s, 1H), 3.52-3.60 (m, 3H), 3.66-3.70 (m, 1H), 4.25-4.27 (d, 1H), 7.18-7.20 (d, 2H), 7.32 (s, 1H), 7.42-7.46 (m, 1H), 7.60 (s, 1H), 7.68-7.70 (m, 1H), 7.74 (s, 1H), 8.32 (s, 1H).

LC-MS-409 (ES, m/z): [M+H]⁺ 555

H-NMR-409 1H NMR (300 MHz, DMSO-d6 ppm) δ 1.60-1.89 (m, 5H), 1.90-2.09 (m, 1H), 2.06-2.13 (m, 2H), 3.18-3.32 (m, 4H), 3.43-3.48 (m, 3H), 3.50 (s, 1H), 3.52-3.60 (m, 3H), 3.66-3.70 (m, 1H), 4.25-4.27 (d, 1H), 7.18-7.20 (d, 2H), 7.32 (s, 1H), 7.42-7.46 (m, 1H), 7.60 (s, 1H), 7.68-7.70 (m, 1H), 7.74 (s, 1H), 8.33 (s, 1H).

### Example 404. Synthesis of Compound 410 and 411

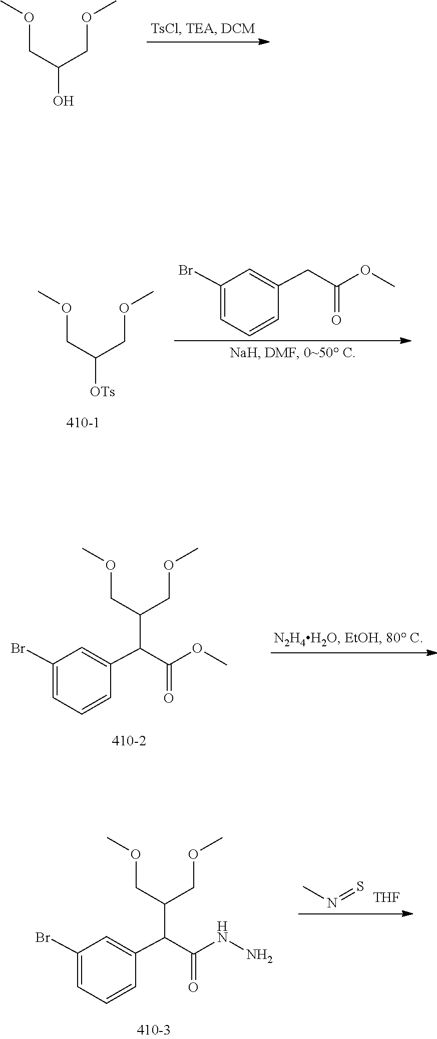

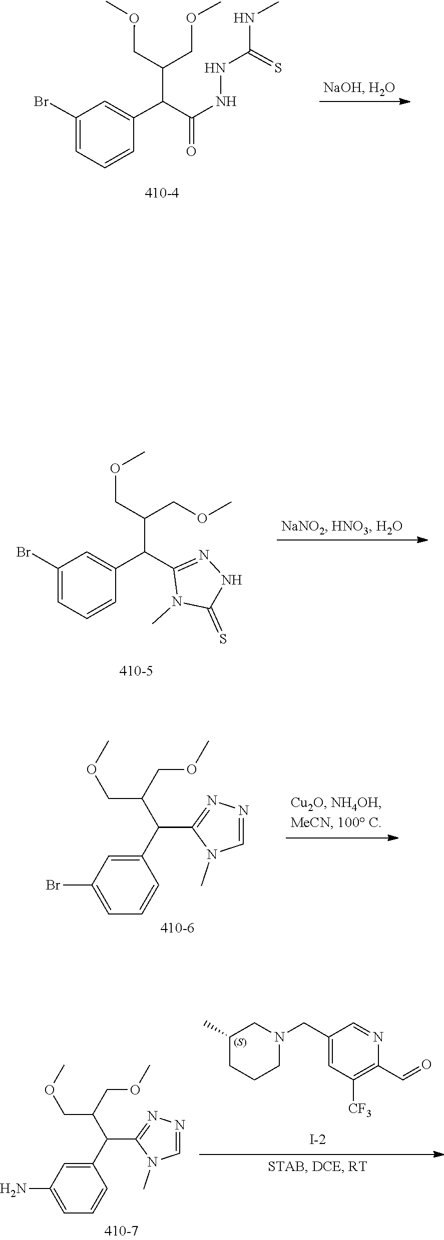

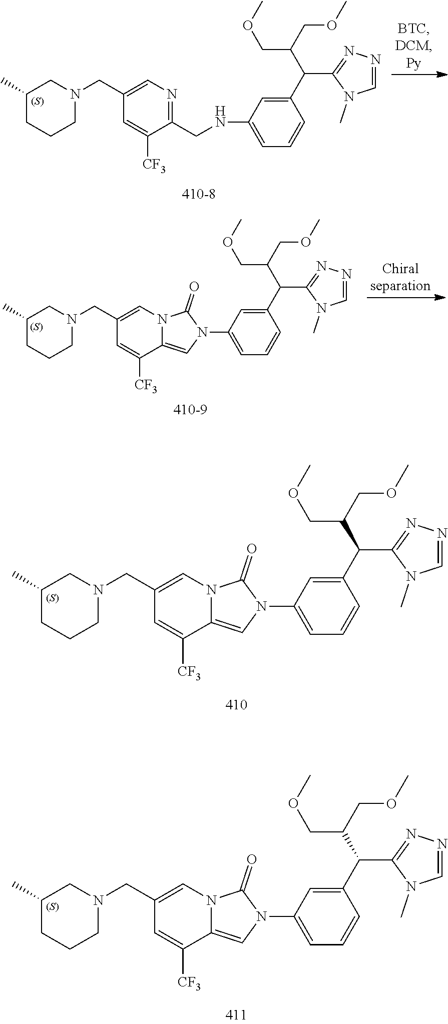

### 1. Synthesis of 410-1

To a stirred mixture of glycerin dimethyl ether (30 g, 249.692 mmol, 1 equiv) and TsCl (95.20 g, 499.384 mmol, 2 equiv) in DCM (300 mL) was added TEA (75.80 g, 749.076 mmol, 3 equiv) dropwise at room temperature. The resulting mixture was stirred overnight at room temperature. The reaction was quenched with water (500 mL) at room temperature. The resulting mixture was extracted with CH₂Cl₂ (3×200 mL). The combined organic layers were dried over anhydrous Na₂SO₄. After filtration, the filtrate was concentrated under reduced pressure. The residue was purified by silica gel column chromatography, eluted with CH₂Cl₂/MeOH (100:1) to afford 410-1 (20 g, 29.20%) as a white solid.

### 2. Synthesis of 410-2

To a stirred mixture of methyl 2-(3-boronophenyl)acetate (25.05 g, 109.354 mmol, 1.50 equiv) and DMF (216.21 mL, 2793.720 mmol, 38.32 equiv) was added NaH (3.50 g, 145.810 mmol, 2 equiv) at 0° C. under nitrogen atmosphere. The resulting mixture was stirred for 30 min at 0° C. under nitrogen atmosphere. To the above mixture was added 410-1 (20 g, 72.905 mmol, 1 equiv) at room temperature. The resulting mixture was stirred overnight at 50° C. The mixture was allowed to cool down to room temperature. The reaction was quenched with saturated NH₄Cl (aq.) (1000 mL) at room temperature. The resulting mixture was extracted with EtOAc (4×300 mL). The combined organic layers were concentrated under reduced pressure. The residue was purified by reverse flash chromatography with the following conditions: column, C18 silica gel; mobile phase, MeCN in Water (0.1% FA), 0% to 100% gradient in 55 min; detector, UV 220 nm. This resulted in methyl 4102-2 (7 g, 28.99%) as a yellow oil.

### 3. Synthesis of 410-3

To a stirred mixture of methyl 410-2 (7 g, 21.135 mmol, 6.9 equiv) in EtOH (70 mL) was added NH₂NH₂·H₂O (1.53 g, 30.630 mmol, 10 equiv) at room temperature. The resulting mixture was stirred overnight at 80° C. under nitrogen atmosphere. The mixture was allowed to cool down to room temperature. The resulting mixture was concentrated under reduced pressure. The residue was purified by silica gel column chromatography, eluted with CH₂Cl₂/MeOH (100:1) to afford 410-3 (6.1 g, 87.14%) as a yellow solid.

### 4. Synthesis of 410-4

To a stirred mixture of 410-3 (3 g, 9.058 mmol, 1 equiv) and tetrahydrofuran (40 mL) was added methyl isothiocyanate (3.31 g, 45.290 mmol, 5 equiv) at room temperature. The resulting mixture was stirred overnight at room temperature. The reaction was quenched with water (80 mL) at room temperature. The aqueous layer was extracted with CH₂Cl₂ (4×40 mL). The resulting mixture was concentrated under vacuum. This resulted in 410-4 (2.6 g, 71.00%) as a yellow solid.

### 5. Synthesis of 410-5

To a stirred mixture of NaOH (1.29 g, 32.219 mmol, 5.01 equiv) in H₂O (100 mL) was added 410-4 (2.6 g, 6.431 mmol, 1 equiv) at room temperature. The resulting mixture was stirred overnight at room temperature. The mixture was neutralized to pH 7 with HCl (1M). The aqueous layer was extracted with CH₂Cl₂ (4×30 mL). The resulting mixture was concentrated under vacuum. The residue was purified by trituration with PE: EA=20:1 (25 mL). This resulted in 410-5 (2.1 g, 84.53%) as a white solid.

### 6. Synthesis of 410-6

To a stirred mixture of 410-6 (2.1 g, 5.436 mmol, 1 equiv) and NaNO2 (1.13 g, 16,308 mmol, 3 equiv) in 1H₂O (40 mL) was added HNO₃ (20 mL, 1 mol/L) dropwise at room temperature. The mixture was neutralized to pH 7 with saturated NaHCO₃ (aq.). The aqueous layer was extracted with CH₂Cl₂ (5×30 mL). The resulting mixture was concentrated under reduced pressure. The residue was purified by silica gel column chromatography, eluted with PE/EA (5:1) to afford 410-6 (1 L-5 g, 77.89%) as a white solid.

### 7. Synthesis of 410-7

Into a pressure tank reactor was added 410-6 (1.45 g, 4,093 mmol, 1 equiv) and Cu₂O (0.59 g, 4.093 mmol, 1 equiv) in NH₄OH (0.75 mL) and MeCN (0.75 mL) at room temperature. The resulting mixture was stirred overnight at 100° C. The mixture was allowed to cool down to room temperature. The resulting mixture was concentrated under vacuum. The residue was purified by Prep-TLC (DCM/MeOH 15:1) to afford 410-7 (600 mg, 50.48%) as a brown solid.

### 8. Synthesis of 410-8

To a stirred mixture of 410-7 (600.00 mg, 2.066 mmol, 1 equiv) and 1-2 (650.75 mg, 2.273 mmol, 1.1 equiv) in DCE (10 mL) was added STAB (875.88 mg, 4.132 mmol, 2 equiv) at room temperature. The resulting mixture was stirred overnight at room temperature. The reaction was quenched with NaHCO₃ (aq.) (80 mL) at room temperature. The aqueous layer was extracted with CH₂Cl₂ (3×30 mL). The resulting mixture was concentrated under reduced pressure. The residue was purified by Prep-TLC (CH₂Cl₂/MeOH 20:1) to afford 410-8 (900 mg, 77.68%) as a yellow solid.

### 9. Synthesis of 410-9

To a stirred mixture of 410-8 (600 mg, 1.070 mmol, 1 equiv) and Pyridine (507.90 mg, 6.420 mmol, 6 equiv) in DCE (6 mL) was added Triphosgene (123.85 mg, 0.417 mmol, 0.39 equiv) at 0° C. The resulting mixture was stirred for 10 min at room temperature. The reaction was quenched with NaHCO₃ (aq.) (80 mL) at room temperature. The aqueous layer was extracted with CH₂Cl₂ (3×30 mL). The resulting mixture was concentrated under reduced pressure. The residue was purified by Prep-TLC (CH₂Cl₂/MeOH 20:1) to afford 410-9 (360 mg, 57.34%) as a yellow solid.

### 10. Synthesis of 410 and 411

The 410-9 (300 mg) was purified by Prep-Chiral-HPLC with the following conditions (Column: Chiralpak IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 18 min; Wave Length: 220/254 nm; RT1 (min): 8.14; RT2 (min): 13.09; the first peak is 411; the second peak is 410) to afford 410 (128.8 mg, 42.93%) as a yellow solid.

LC-MS-411: (ES, m/z): [M+H]⁺ 558. H-NMR-411: (400 MHz, DMSO-d₆, δ ppm): 0.80-0.91 (m, 4H), 1.44-1.98 (m, 6H), 2.74-2.83 (m, 3H), 3.10-3.14 (m, 1H), 3.17 (s, 3H), 3.24 (s, 3H), 3.26-3.30 (m, 3H), 3.33-3.40 (m, 2H), 3.41 (s, 3H), 4.26-4.29 (d, 1H), 7.02 (s, 1H), 7.32-7.34 (m, 2H), 7.45-7.48 (m, 1H), 7.66-7.70 (m, 1H), 7.74-7.76 (d, 1H), 7.83 (s, 1H), 8.34 (s, 1H).

LC-MS-410: (ES, m/z): [M+H]⁺ 558. H-NMR-410: (400 MHz, DMSO-d6, δ ppm): 0.80-0.91 (m, 4H), 1.44-1.98 (m, 6H), 2.74-2.83 (m, 3H), 3.10-3.14 (m, 1H), 3.17 (s, 3H), 3.24 (s, 3H), 3.26-3.30 (m, 3H), 3.33-3.40 (m, 2H), 3.41 (s, 3H), 4.26-4.29 (d, 1H), 7.02 (s, 1H), 7.32-7.34 (m, 2H), 7.45-7.48 (m, 1H), 7.66-7.70 (m, 1H), 7.74-7.76 (d, 1H), 7.83 (s, 1H), 8.34 (s, 1H).

### Example 405. Synthesis of Compound 412

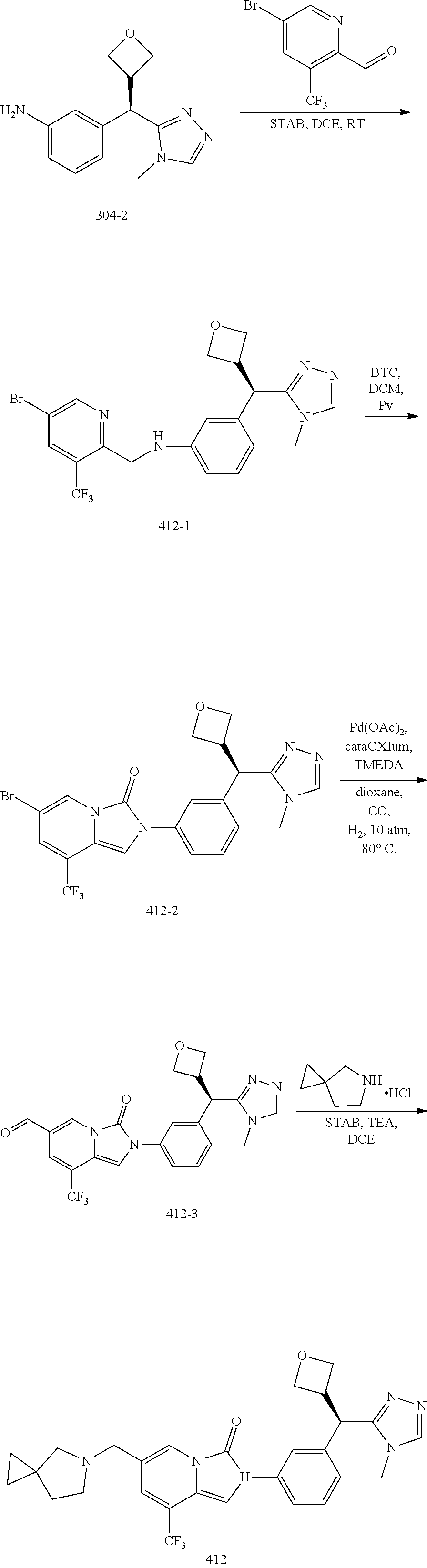

### 1. Synthesis of 412-1

To a stirred solution of 304-2 (3.4 g, 13.917 mmol, 1 equiv) and 5-bromo-3-(trifluoromethyl)picolinaldehyde (4.24 g, 16.700 mmol, 1:2 equiv) in DCE (100 mL) was added STAB (5.90 g, 27.834 mmol, 2 equiv) at room temperature. The resulting mixture was stirred overnight at room temperature. The reaction was quenched with saturated NaHCO₃ (aq.) (300 mL) at room temperature. The aqueous layer was extracted with DCM/MeOH=10:1 (3×200 mL). The resulting mixture was concentrated under vacuum. The residue was purified by silica gel column chromatography, eluted with DCM/MeOH (20:1) to afford 412-1 (4 g, 57.80%) as a Brown yellow solid.

### 2. Synthesis of 412-2

To a stirred solution of 412-2 (4 g, 8.294 mmol, 1 equiv) and Pyridine (6.56 g, 82.933 mmol, 10.00 equiv) in DCM (100 mL) was added Triphosgene (0.98 g, 3.318 mmol, 0.40 equiv) at 0° C. The resulting mixture was stirred for 10 min at room temperature. The reaction was quenched with saturated NaHCO₃ (aq.) (200 mL) at room temperature. The aqueous layer was extracted with DCM (2×200 mL). The resulting mixture was concentrated under vacuum. The residue was purified by trituration with MTBE (100 mL). This resulted in 412-2 (3.5 g, 75.55%) as a reddish brown solid.

### 3. Synthesis of 412-3

To a solution of 412-2 (3.4 g, 6.689 m mol, 1 equiv) in dioxane (100 mL) was added Pd(OAc)₂ (0.15 g, 0.669 mmol, 0.1 equiv) and bis(adamantan-1I-yl)(butyl)phosphane (0.48 g, 1.338 mmol, 0.2 equiv) in a pressure tank. The mixture was purged with nitrogen for 3 min and then was pressurized to 15 atm with CO/H₂=1:1 at 80° C. for overnight. The reaction mixture was cooled to room temperature. The resulting mixture was concentrated under vacuum. The residue was purified by silica gel column chromatography, eluted with DCM/MeOH (20:1) to afford 412-2 (1.1 g, 32.36%) as a reddish brown solid.

### 4. Synthesis of 412

To a stirred solution of 412-3 (250 mg, 0.547 mmol, 1 equiv) and 5-azaspiro[2.4]heptane hydrochloride (219.09 mg, 1.641 mmol, 3 equiv) in DCE (5 mL) was added TEA (165.92 mg, 1.641 mmol, 3 equiv) at room temperature. The resulting mixture was stirred for 1 h at room temperature. To the above mixture was added STAB (231.67 mg, 1.094 mmol, 2 equiv) at room temperature. The resulting mixture was stirred overnight at room temperature. The reaction was quenched with saturated NH4Cl (aq.) (50 mL) at room temperature. The aqueous layer was extracted with DCM (2×30 mL). The resulting mixture was concentrated under vacuum. The residue was purified by Prep-TLC (DCM/MeOH 20:1) to afford the crude product. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 33% B to 46% B in 8 min; Wave Length: 220 nm; RT1 (min): 7.07) to afford 412 (24.9 mg, 8.33%) as a yellow solid,

LC-MS-412: (ES, m/z): [M+H]⁺ 539. H-NMR-412: (400 MHz, DMSO-d6, δ ppm): 0.41-0.63 (d, 4H), 1.71-1.81 (m, 2H), 2.47-2.49 (m, 2H), 2.67-2.71 (m, 2H), 3.38-3.41 (m, 5H), 3.89-3.95 (m, 1H), 4.27-4.30 (m, 1H), 4.48-4.49 (d, 2H), 4.72-4.80 (m, 2H), 7.04 (s, 1H), 7.17-7.19 (d, 1H), 7.37 (s, 1H), 7.45-7.49 (m, 1H), 7.67 (s, 1H), 7.75-7.76 (m, 2H), 8.37 (s, 1H).

### Example 406. Synthesis of Compounds 413-415

The following compounds were prepared in a similar manner to 412-0, replacing and 5-azaspiro[2.4]heptane hydrochloride with the appropriate amine.

| Compound | Yield and Characterization Data |
|---|---|
| 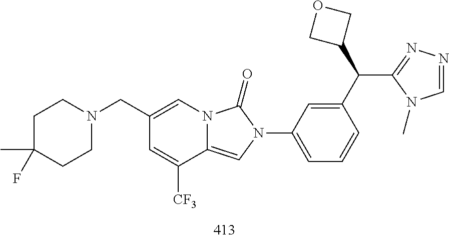 [SMILES: Cn1cnnc1[C@@H](c1cccc(-n2cc3c(C(F)(F)F)cc(CN4CCC(C)(F)CC4)cn3c2=O)c1)C1COC1] | 8.88% yield (ES, m/z): [M + H]⁺ 559 (400 MHz, DMSO-d₆, δ ppm): 1.28-1.37 (m, 4H), 1.66-1.70 (m, 1H), 1.70-1.81 (m, 3H), 2.24-2.29 (m, 2H), 2.59-2.62 (m, 2H), 3.30-3.32 (m, 2H), 3.38 (s, 3H), 3.89-3.95 (m, 1H), 4.27-4.30 (m, 1H), 4.48-4.51 (d, 2H), 4.72-4.81 (m, 2H), 7.01 (s, 1H), 7.17-7.19 (d, 1H), 7.37 (d, 1H), 7.45-7.49 (m,1H), 7.67 (s, 1H), 7.75-7.77 (m, 2H), 8.37 (s, 1H). |
| 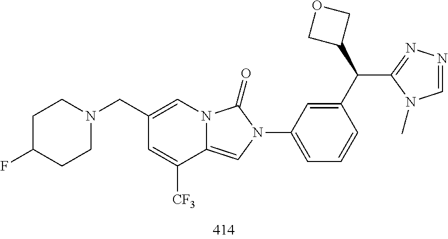 [SMILES: Cn1cnnc1[C@@H](c1cccc(-n2cc3c(C(F)(F)F)cc(CN4CCC(F)CC4)cn3c2=O)c1)C1COC1] | 14.01% yield (ES, m/z): [M + H]⁺ 545 (400 MHz, DMSO-d₆, δ ppm): 1.61-1.79 (m, 2H), 1.80-1.91 (m, 2H), 2.34-2.36 (m, 2H), 2.50-2.54 (m, 2H), 3.30-3.32 (m, 2H), 3.38 (s, 3H), 3.89-3.95 (m, 1H), 4.27-4.30 (m, 1H), 4.48-4.51 (d, 2H), 4.62-4.81 (m, 3H), 7.01 (s, 1H), 7.17-7.19 (d, 1H), 7.37 (d, 1H), 7.45-7.49 (m,1H), 7.67 (s, 1H), 7.75-7.77 (m, 2H), 8.37 (s, 1H). |
| 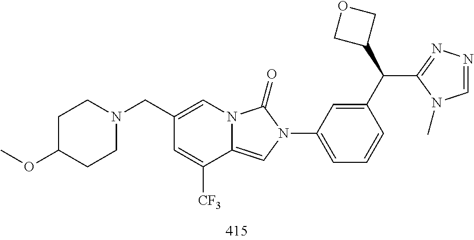 [SMILES: COC1CCN(Cc2cc(C(F)(F)F)c3cn(-c4cccc([C@H](c5nncn5C)C5COC5)c4)c(=O)n3c2)CC1] | 24.91% yield (ES, m/z): [M + H]⁺ 557 (400 MHz, DMSO-d6, δ ppm): 1.35-1.47 (m, 2H), 1.74-1.89 (m, 2H), 2.05-2.15 (m, 2H), 2.60-2.66 (m, 2H), 3.09-3.22 (m, 4H), 3.27 (s, 2H), 3.38 (s, 3H), 3.89-3.93 (m, 1H), 4.27-4.30 (m, 1H), 4.48-4.49 (d, 2H), 4.72-4.80 (m, 2H), 7.00 (s, 1H), 7.17-7.18 (d, 1H), 7.37 (s, 1H), 7.45-7.48 (m, 1H), 7.65 (s, 1H), 7.75-7.76 (m, 2H), 8.37 (s, 1H). |

### Example 407. Synthesis of Compound 416

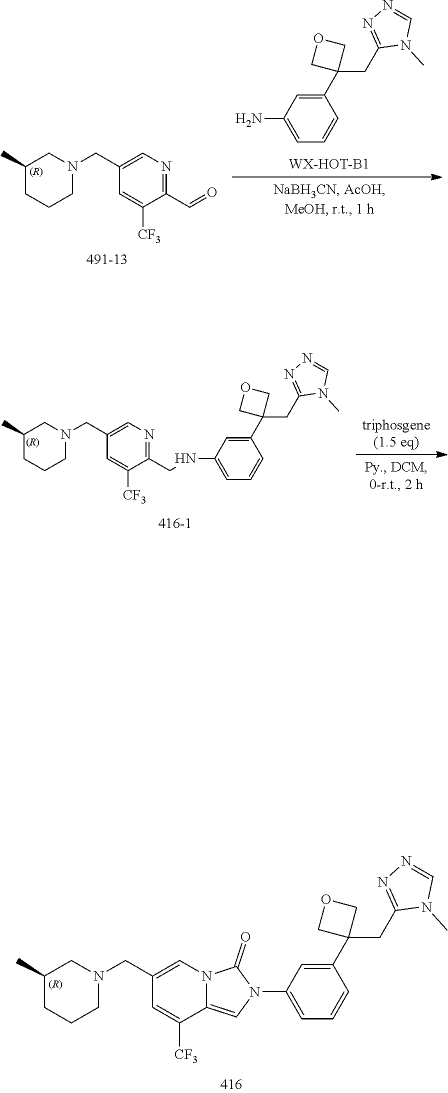

### 1. Synthesis of 416-1

To a solution of 491-13 (554 mg, 2.27 mmol, 1 equiv) and 491-13 (520 mg, 1.82 mmol, 0.8 equiv) in MeOH (10 mL) was added HOAc (130 uL, 2.27 mmol, 1 equiv). The mixture was stirred at 25° C. for 1 h under nitrogen atmosphere. Then NaBH₃CN (142 mg, 2.27 mmol, 1 equiv) was added. The mixture was stirred at 25° C. for 1 h under nitrogen atmosphere. The mixture adjusted pH to 8 by Sat.NaHCO₃ (50 ml) and extracted with EtOAc (20 ml×3). The combined organic layers were dried over anhydrous Na₂SO₄, filtered and the filtrate was concentrated in vacuum. The residue was purified by silica gel column chromatography eluted with DCM/MeOH (10:1) to afford 416-1 (320 mg, 27%) as a yellow solid.

### 2. Synthesis of 416

To a solution of 416-1 (0.13 g, 252 umol, 1 equiv) in DCM (10 mL) was added Py (122 uL, 1.52 mmol, 6 equiv) and bis(trichloromethyl) carbonate (150 mg, 505 umol, 2 equiv) at 0° C. The mixture was stirred at 25° C. for 2 h under nitrogen atmosphere. The reaction mixture was diluted with Sat.NaHCO₃ (100 mL), extracted with DCM (50 mL×3). The combined organic layers were dried over anhydrous Na₂SO₄, filtered and the filtrate was concentrated in vacuum. The residue was purified by Prep-HPLC with the following conditions (column: Phenomenex Luna C18 150*40 mm*10 um; Mobile Phase A: water (NH₃H2O+NH₄HCO₃, Mobile Phase B: ACN; Flow rate: 50 mL/min; Gradient: 1% B to 35% B in 8 min; Wave Length: 220 nm; RT1 (min): 7.2) to afford 416 (32 mg, 24%) as a yellow solid.

MS-416: (ES, m/z): [M+H]⁺ 541.2. ¹H-NMR-416: (400 MHz, METHANOL-d4) δ 8.21 (s, 1H), 7.68-7.62 (m, 2H), 7.47 (t, J=8.0 Hz, 1H), 7.32 (t, J=1.6 Hz, 1H), 7.12 (d, J=4.0 Hz, 2H), 6.90 (d, J=8.0 Hz, 1H), 5.09 (s, 5H), 3.68 (s, 2H), 2.96 (s, 3H), 2.92-2.84 (m, 2H), 2.03-1.94 (m, 1H), 1.79-1.57 (m, 6H), 1.01-0.93 (m, 1H), 0.91 (d, J=5.6 Hz, 3H).

### Example 408. Synthesis of Compound 417 and 418

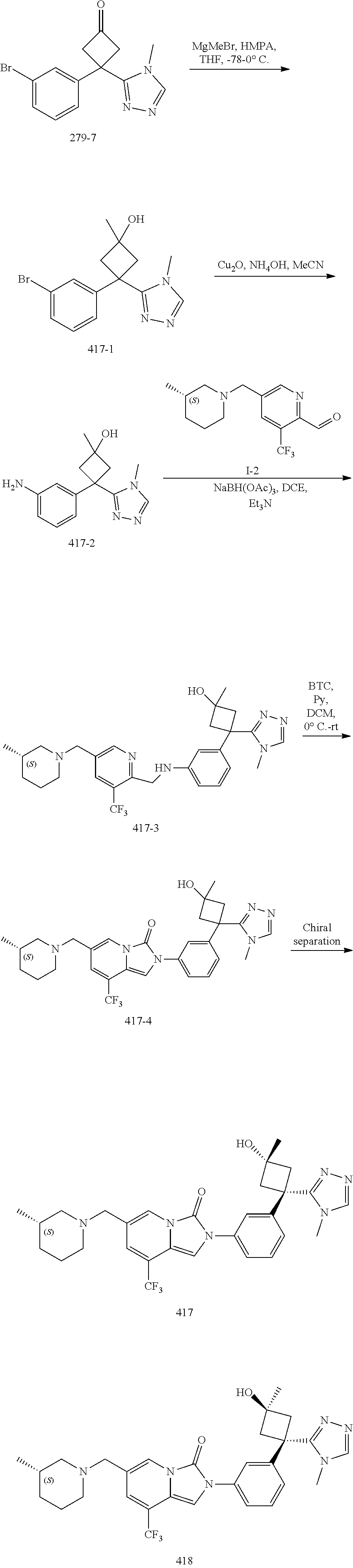

### 1. Synthesis of 417-1

To a stirred solution of 279-7 (10 g, 32.66 mmol, 1.0 equiv) and MeMgBr (11.7 g, 97.98 mmol, 3.0 equiv) in THF (100 mL) was added HMPA (17.6 g, 97.98 mmol, 3.0 equiv) at −78° C. The resulting mixture was stirred for 3 h at −78° C. The reaction was quenched by the addition of sat. NH₄Cl (aq.) (200 mL) at room temperature. The aqueous layer was extracted with EtOAc (3×100 mL). The resulting mixture was concentrated under reduced pressure. The residue was purified by silica gel column chromatography, eluted with CH₂Cl₂/MeOH=20:1 to afford 417-1 (4.5 g, 43%) as an off-white solid.

### 2. Synthesis of 417-2

To a stirred solution of 417-1 (2.4 g, 7.44 mmol, 1.0 equiv) and Cu₂O (0.43 g, 2.98 mmol, 0.4 equiv) in MeCN (15 mL) was added NH₃·H₂O (15 mL) at room temperature. The resulting mixture was stirred for overnight at 100° C. The resulting mixture was concentrated under reduced pressure. The residue was purified by silica gel column chromatography, eluted with CH₂Cl₂/MeOH=20:1 to afford 417-2 (1.6 g, 77%) as a colorless oil.

### 3. Synthesis of 417-3

To a stirred solution of 417-2 (1 g, 3.87 mmol, 1.0 equiv) and I-2 (1.1 g, 3.87 mmol, 1.0 equiv) in DCE (10 mL) was added Et₃N (392 mg, 3.87 mmol, 1.0 equiv) at room temperature. The resulting mixture was stirred for 1 h at room temperature. To the above mixture was added NaBH(OAc)₃ (1.6 g, 7.74 mmol, 2.0 equiv). The resulting mixture was stirred for additional overnight at room temperature. The reaction was quenched by the addition of water (20 mL) at room temperature. The aqueous layer was extracted with CH₂Cl₂ (3×20 mL). The resulting mixture was concentrated under reduced pressure. The residue was purified by Prep-TLC (CH2Cl2/MeOH=10:1) to afford 417-3 (630 mg, 31%) as an off-white solid.

### 4. Synthesis of 417-4

To a stirred solution of 417-3 (630 mg, 1.19 mmol, 1.0 equiv) and pyridine (565 mg, 7.15 mmol, 6.0 equiv) in DCM (7 mL) was added triphosgene (124 mg, 0.41 mmol, 0.35 equiv) at 0° C. The resulting mixture was stirred for 1 h at 0° C. The reaction was quenched by the addition of sat. NaHCO₃ (aq.) (20 mL) at room temperature. The aqueous layer was extracted with CH₂Cl₂ (3×15 mL). The resulting mixture was concentrated under reduced pressure. The residue was purified by Prep-TLC (CH₂Cl₂/MeOH=10:1) to afford 417-4 (220 mg, 33%) as a yellow solid.

### 5. Synthesis of 417 and 418

The 417-4 (220 mg) was purified by chiral separation with the following conditions (Column: CHIRALPAK IC, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 11 min; Wave Length: 220/254 nm; RT1 (min): 5.24; RT2 (min): 7.90; The first peak was the 417-0; the second peak was 418-0. Sample Solvent: EtOH:DCM=1:1-HPLC; Injection Volume: 1 mL; Number of Runs: 4) to afford 417 (54 mg, 25%) as a yellow solid and 418 (54 mg, 25%) as a yellow solid.

LC-MS-417: (ES, m/z): [M+H]⁺ 555. H-NMR-417 (400 MHz, DMSO, δ ppm): 0.85 (s, 4H), 1.12 (s, 3H), 1.40-1.50 (m, 1H), 1.58-1.68 (m, 4H), 1.87-1.92 (t, 1H), 2.74-2.77 (m, 2H), 2.92-2.95 (m, 2H), 3.05-3.08 (m, 2H), 3.17-3.25 (m, 2H), 3.32 (s, 3H), 5.10-5.12 (d, 1H), 7.00 (s, 1H), 7.34-7.36 (m, 2H), 7.47-7.51 (t, 1H), 7.60-7.62 (d, 1H), 7.66 (s, 1H), 7.91 (s, 1H), 8.24 (s, 1H).

LC-MS-418: (ES, m/z): [M+H]+ 555. H-NMR-418: (400 MHz, DMSO, δ ppm): 0.82-0.85 (m, 4H), 1.13 (s, 3H), 1.40-1.49 (m, 1H), 1.58-1.66 (m, 4H), 1.86-1.91 (t, 1H), 2.72-2.85 (m, 4H), 3.03-3.10 (m, 2H), 3.16-3.24 (m, 2H), 3.32 (s, 3H), 5.08-5.12 (d, 1H), 7.00 (s, 1H), 7.14-7.16 (d, 1H), 7.32 (s, 1H), 7.45-7.49 (t, 1H), 7.60-7.70 (m, 3H), 8.28 (s, 1H).

### Example 409. Synthesis of Compound 419 and 420

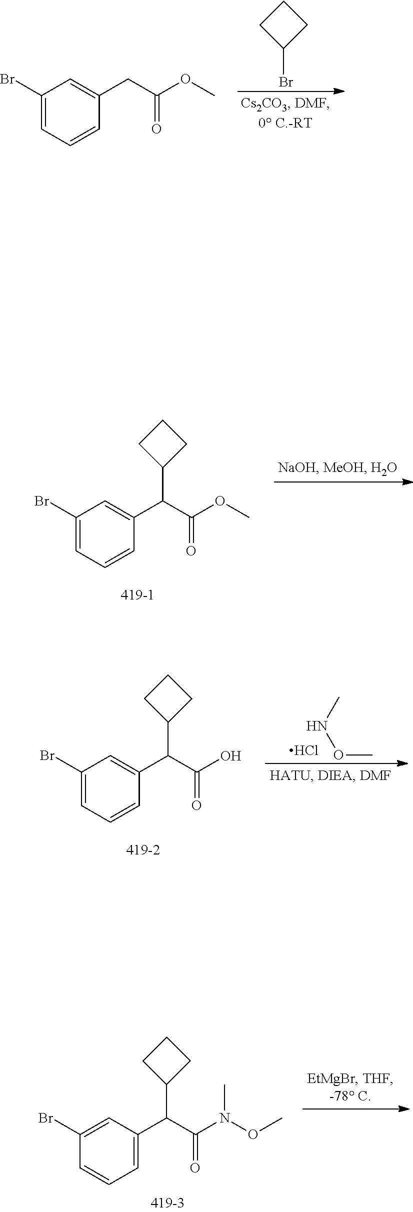

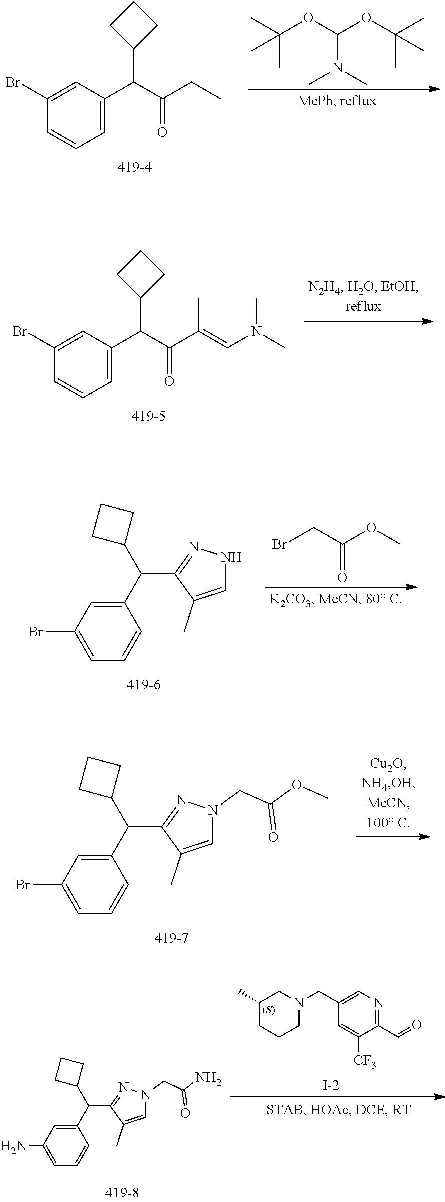

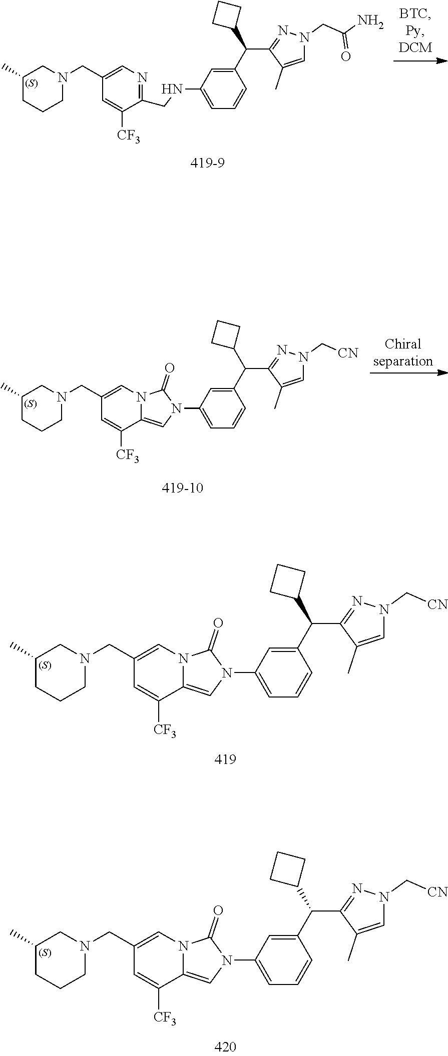

### 1. Synthesis of 419-1

A solution of methyl 2-(3-bromophenyl)acetate (100 g, 436.542 mmol, 1 equiv) in DMF (1000 mL) was treated with Cs₂CO₃ (166.17 g, 2182.710 mmol, 5 equiv) for 3 h at 0° C. under nitrogen atmosphere followed by the addition of bromocyclobutane (176.80 g, 1309.626 mmol, 3 equiv). The resulting mixture was stirred for 20 h at room temperature. The reaction was quenched with water (3000 ml) at room temperature. The aqueous layer was extracted with EtOAc (2×2 L). The resulting mixture was concentrated under reduced pressure. The residue was purified by silica gel column chromatography, eluted with PE/EA (10:1) to afford methyl 419-1 (94 g, 76.04%) as a yellow solid.

### 2. Synthesis of 419-2

A solution of methyl 419-1 (94 g, 331.962 mmol, 1 equiv) in MeOH (3000 mL) was treated with NaOH (995.89 mL, 995.886 mmol, 3 equiv, 1M). The resulting mixture was stirred for 2 h at room temperature. The MeOH was removed under reduced pressure. The mixture was acidified to pH 5 with HCl (1 M). The precipitated solids were collected by filtration and washed with water (150 mL). This resulted in 419-2 (80 g, 89.54%) as a yellow oil.

### 3. Synthesis of 419-3

To a stirred mixture of 419-2 (80 g, 297.245 mmol, 1 equiv), DIEA (115.25 g, 891.735 mmol, 3 equiv) and methoxy(methyl)amine hydrochloride (57.99 g, 594.490 mmol, 2 equiv) in DMF (800 mL) was added HATU (124.33 g, 326.970 mmol, 1.1 equiv). The resulting mixture was stirred for 12 h at room temperature. The reaction was quenched with water (2500 ml) at room temperature. The aqueous layer was extracted with EtOAc (2 L). The resulting mixture was concentrated under reduced pressure. The residue was purified by silica gel column chromatography, eluted with PE/EA (12:1) to afford 419-3 (63 g, 59.06%) as a yellow solid.

### 4. Synthesis of 419-4

To a stirred solution of 419-3 (63 g, 201.789 mmol, 1 equiv) in THF (600 mL) was added EtMgBr (807 mL, 807.156 mmol, 4 equiv) at −78° C. The resulting mixture stirred for 5 h at room temperature. The reaction was quenched with sat. NH₄Cl (aq.)(2000 ml) at room temperature. The aqueous layer was extracted with EtOAc (3×800 mL). The resulting mixture was concentrated under reduced pressure. The residue was purified by silica gel column chromatography, eluted with PE/EA (5:1) to afford 419-4 (42.4 g, 66.7%) as a yellow oil.

### 5. Synthesis of 419-5

A solution of 419-4 (28.5 g, 101.354 mmol, 1 equiv) in toluene (300 mL) was treated with [bis(tert-butoxy)methyl]dimethylamine (51.52 g, 253.385 mmol, 2.5 equiv). The resulting mixture was stirred for 5 h at room temperature. The resulting mixture was concentrated under vacuum to afford 419-6 (38.3 g, crude) as a yellow oil.

### 6. Synthesis of 419-6

A solution of 419-5 (38.5 g, 114.490 mmol, 1 equiv) in EtOH (385 mL) was treated for hydrazine (36.69 g, 1144.900 mmol, 10 equiv). The resulting mixture was stirred for 8 h at 80° C. The reaction was quenched by the addition of water (1000 mL) at room temperature. The aqueous layer was extracted with CH₂Cl₂/MeOH (10:1) (3×1000 mL). The resulting mixture was concentrated under reduced pressure. The residue was purified by reverse flash chromatography with the following conditions: column, C18 silica gel; mobile phase, MeCN in water (0.1% NH₄HCO₃), 25% to 85% gradient in 30 min; detector, UV 220 nm. This resulted in 419-6 (15 g, 37.77%) as a yellow solid.

### 7. Synthesis of 419-7

A solution of 419-6 (3 g, 9.829 mmol, 1 equiv) in MeCN (30 mL) was treated for methyl 2-bromoacetate (4.51 g, 29.487 mmol, 3 equiv) and K₂CO₃ (2.72 g, 19.658 mmol, 2 equiv), The resulting mixture was stirred for 4 h at 80° C. The resulting mixture was filtered, the filter cake was washed with MeCN (2×20 mL). The filtrate was concentrated under reduced pressure. The residue was purified by silica gel column chromatography, eluted with PE/EA (5:1) to afford 419-7 (2.92 g, 72.44%) as a light yellow solid.

### 8. Synthesis of 419-8

To a solution of methyl 419-7 (2.86 g, 7.581 mmol, 1 equiv) in NH₃ (7M in MeOH) (100 mL) was added Cu2O (0.43 g, 3.032 mmol, 0.4 equiv) in a pressure tank. The resulting mixture was stirred for overnight at 100° C. The resulting mixture was filtered, the filter cake was washed with MeOH (10 mL). The filtrate was concentrated under reduced pressure. The residue was purified by reverse flash chromatography with the following conditions: column, C18 silica gel; mobile phase, MeCN in Water (0.1% NH₄HCO₃), 20% to 70% gradient in 30 min; detector, UV220 nm to afford 419-8 (568 mg, 22.60%) as a white solid.

### 9. Synthesis of 419-9

A solution of 419-8 in DCE (6 mL, 75.796 mmol, 39.82 equiv) was treated with I-2 (599.48 mg, 2.094 mmol, 1.1 equiv) for 2 h at room temperature under nitrogen atmosphere followed by the addition of STAB (605.16 mg, 2.856 mmol, 1.5 equiv) at room temperature. The resulting mixture was stirred for overnight at room temperature under nitrogen atmosphere. The reaction was quenched with sat. NaHCO₃ (aq.)(20 ml) at room temperature. The resulting mixture was extracted with CH2Cl2/MeOH (10:1)(2×30 mL). The combined organic layers were concentrated under reduced pressure. The residue was purified by reverse flash chromatography with the following conditions: column, C18 silica gel; mobile phase, MeCN in Water (0.1% NH₄HCO₃), 20% to 80% gradient in 30 min; detector, UV 254 nm to afford 419-9 (1.13 g, 62.63%) as a yellow solid.

### 10. Synthesis of 419-10

A solution of 419-9 (1.13 g, 1.987 mmol, 1 equiv) in DCM (12 mL) was treated with Pyridine (943.04 mg, 11.922 mmol, 6 equiv) at room temperature followed by the addition of Triphosgene (17.72 mg, 0.060 mmol, 0.4 equiv). The resulting mixture was stirred for 30 min at room temperature under nitrogen atmosphere. The reaction was quenched by the addition of NaHCO₃ (20 mL) at room temperature. The resulting mixture was extracted with DCM/MeOH (10:1)(2×40 mL). After filtration, the filtrate was concentrated under reduced pressure. The residue was purified by reverse flash chromatography with the following conditions: column, silica gel; mobile phase, MeCN in water (0.1% NH₄HCO₃), 20% to 80% gradient in 30 min; detector, UV 254 nm. This resulted in 419-10 as a yellow solid.

### 11. Synthesis of 419 and 420

The 419-10 (350 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: ACN (0.1% IPAmine); Flow rate: 20 mL/min; Gradient: 20% B to 20% B in 16 min; Wave Length: 220/254 nm; RT1 (min): 8.93; RT2 (min): 11.44; the first peak was 419-0; the second peak was 420-0) to afford 419 (104.2 mg, 29.77%) as a yellow solid and 420 (110.8 mg, 30.80%) as a yellow solid.

LC-MS-419: (ES, m/z): [M+H]⁺ 577. H-NMR-419: (400 MHz, DMSO, δ ppm): 0.83-0.84 (m, 4H), 1.42-1.53 (m, 1H), 1.58-1.61 (m, 5H), 1.62-1.84 (m, 4H), 1.86 (s, 3H), 1.88-1.98 (m, 1H), 1.99-2.10 (m, 1H), 2.77-2.85 (m, 2H), 3.11-3.17 (m, 1H), 3.27 (s, 2H), 3.99-4.01 (d, 1H), 5.36 (s, 2H), 7.00 (s, 1H), 7.27-7.28 (d, 1H), 7.30 (s, 1H), 7.39-7.42 (m, 1H), 7.47 (s, 1H), 7.65-7.66 (m, 1H), 7.67 (s, 1H), 7.73 (s, 1H).

LC-MS-420: (ES, m/z): [M+H]⁺ 577. H-NMR-420: (400 MHz, DMSO, δ ppm): 0.83-0.84 (m, 4H), 1.42-1.53 (m, 1H), 1.58-1.62 (m, 5H), 1.64-1.87 (m, 4H), 1.86-1.98 (m, 4H), 1.99-2.10 (m, 1H), 2.75-2.85 (m, 2H), 3.11-3.17 (m, 1H), 3.27 (s, 2H), 3.99-4.01 (d, 1H), 5.36 (s, 2H), 7.01 (s, 1H), 7.21-7.22 (d, 1H), 7.28 (s, 1H), 7.38-7.42 (t, 1H), 7.47 (s, 1H), 7.61-7.66 (m, 1H), 7.67 (s, 1H), 7.74 (s, 1H).

### Example 410. Synthesis of Compound 421 and 422

### 1. Synthesis of 421-1

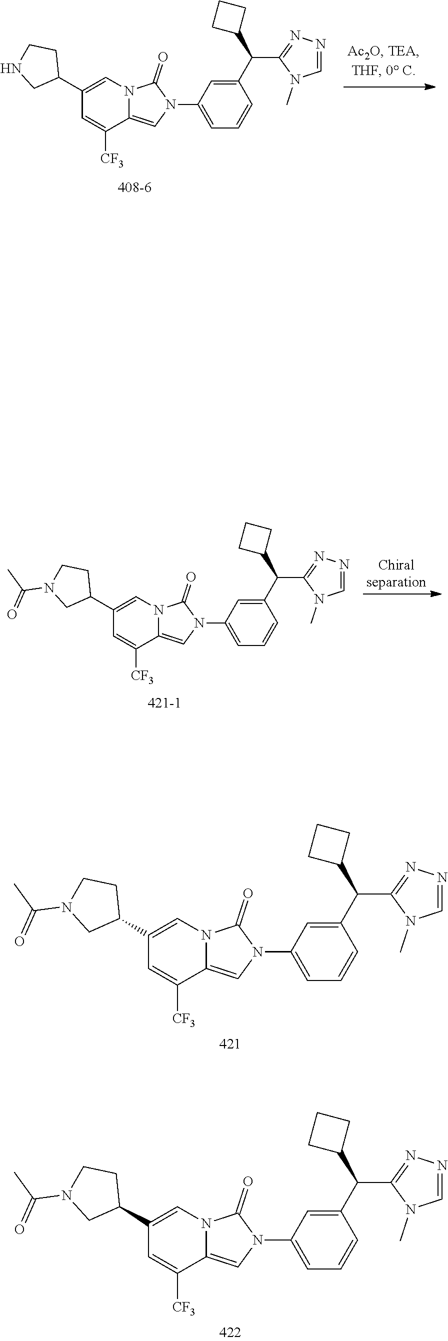 [SMILES: Cn1cnnc1[C@@H](c1cccc(-n2cc3c(C(F)(F)F)cc(C4CCNC4)cn3c2=O)c1)C1CCC1 ; CC(=O)N1CCC(c2cc(C(F)(F)F)c3cn(-c4cccc([C@H](c5nncn5C)C5CCC5)c4)c(=O)n3c2)C1 ; CC(=O)N1CC[C@H](c2cc(C(F)(F)F)c3cn(-c4cccc([C@H](c5nncn5C)C5CCC5)c4)c(=O)n3c2)C1 ; CC(=O)N1CC[C@@H](c2cc(C(F)(F)F)c3cn(-c4cccc([C@H](c5nncn5C)C5CCC5)c4)c(=O)n3c2)C1]

Into a 100 mL 3-necked round-bottom flask were added 408-6 (230 mg, 0.463 mmol, 1 equiv), THF (5 mL), Ac₂O (94.58 mg, 0.926 mmol, 2 equiv) and TEA (140.62 mg, 1.389 mmol, 3 equiv) at 0° C. The resulting mixture was stirred for 4 h at 0° C. The reaction was quenched by the addition of Water (10 mL) at room temperature. The aqueous layer was extracted with CH₂Cl₂ (3×10 mL). The resulting mixture was concentrated under reduced pressure. The residue was purified by Prep-TLC eluted with CH₂Cl₂/MeOH (10:1) to afford 421-1 (200 mg, 80.17%) as a light yellow solid.

### 2. Synthesis of 421 and 422

The 421-1 (200 mg) was purified by Chiral separation with the following conditions (Column: Exsil Chiral-NR, 3*25 cm, 8 μm; Mobile Phase A: CO₂, Mobile Phase B: MeOH: ACN=: 1 (0.1% 2M NH₃-MeOH); Flow rate: 100 mL/min; Gradient: isocratic 45% B; Column Temperature (35° C.); Back Pressure (bar): 100; Wave Length: 220 nm; RT1 (min): 8.33; RT2 (min): 9.93; The first peak was 421-0; the second peak was 421-0. Sample Solvent: MeOH-Preparative; Injection Volume: 4 mL; Number of Runs: 8) to afford crude product (60 ng). The residue was purified by Prep-TLC eluted with CH₂Cl₂/MeOH (10:1) to afford 421 (46.5 mg, 23.25%) as a light yellow solid and 422 (47.2 mg, 23.60%) as a light yellow solid.

LC-MS-421: (ES, m/z): [M+H]⁺ 539. H-NMR-421: 1H NMR (300 MHz, DMSO-d6 ppm) S 1.61-1.89 (m, 5H), 1.90-2.28 (m, 6H), 3.15-3.25 (m, 3H), 3.35-3.41 (m, 4H), 3.50-3.81 (m, 2H), 4.25-4.27 (d, 1H), 7.17-7.18 (m, 2H), 7.32-7.33 (d, 1H), 7.42-7.46 (m, 1H), 7.59-7.74 (m, 3H), 8.33 (s, 1H).

LC-MS-422: (ES, m/z): [M+H]⁺ 539. H-NMR-422: 1H NMR (300 MHz, DMSO-d6 ppm) δ 1.61-1.89 (m, 5H), 1.90-2.28 (m, 6H), 3.15-3.25 (m, 3H), 3.35-3.41 (m, 4H), 3.50-3.61 (m, 1H), 3.61-3.81 (m, 1H), 4.25-4.27 (d, 1H), 7.17-7.18 (m, 2H), 7.32-7.33 (d, 1H), 7.42-7.46 (m, 1H), 7.59-7.74 (m, 3H), 8.33 (s, 1H).

### Example 411. Synthesis of Compound 423

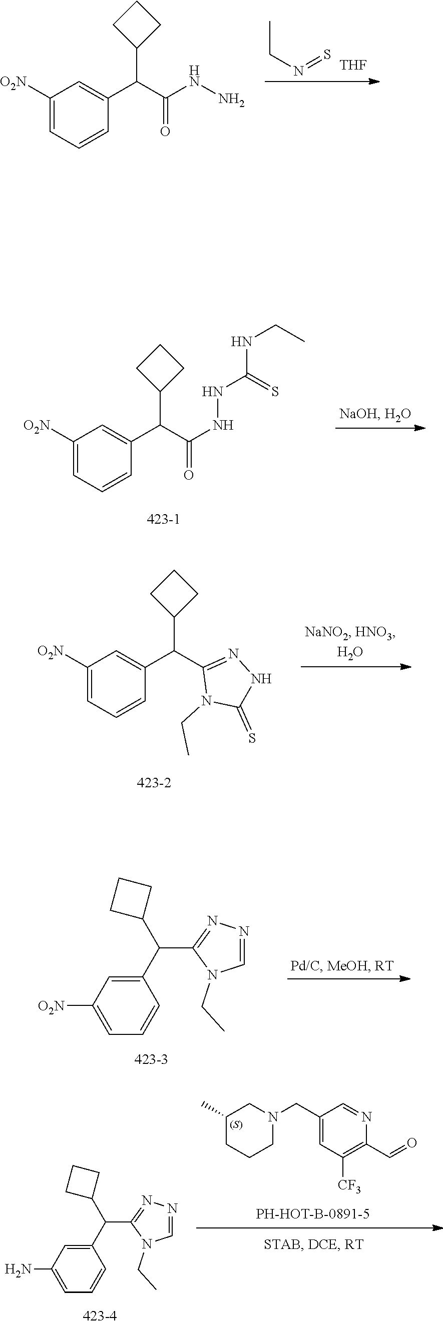

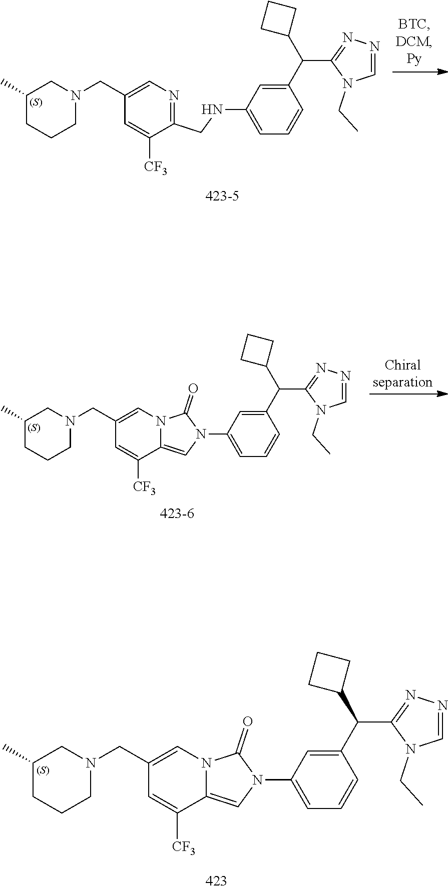

### 1. Synthesis of 423-1

To a stirred solution of 2-cyclobutyl-2-(3-nitrophenyl)acetohydrazide (10 g, 40.117 mmol, 1.00 equiv) in tetrahydrofuran (200 mL) was added isothiocyanatoethane (10.49 g, 120.351 mmol, 3 equiv) at room temperature. The resulting mixture was stirred overnight at room temperature. The resulting mixture was diluted with water (150 mL). The precipitated solids were collected by filtration and washed with water (20 ml). This resulted in 423-1 (12 g, 91%) as an off-white solid.

### 2. Synthesis of 423-2

To a stirred solution of 423-1 (12 g, 35.671 mmol, 1 equiv) in H2O (120.00 mL) was added NaOH (14.27 g, 356,710 mmol, 10 equiv) at room temperature. The resulting mixture was stirred overnight at 50° C. The mixture was neutralized to pH 7 with HC (aq). The precipitated solids were collected by filtration and washed with water (4×50 ml). This resulted in 423-2 (11 g, 96.85%) as a white solid,

### 3. Synthesis of 423-3

To a stirred solution of 423-2 and NaNO2 (23.84 g, 345.480 mmol, 10 equiv) in H2O (440.00 mL) was added HNO₃ (21.77 g, 345.480 mmol, 10 equiv) at room temperature. The resulting mixture was stirred overnight at room temperature. The mixture was basified to pH 7 with saturated NaHCO₃ (aq.). The precipitated solids were collected by filtration and washed with water (3×50 m L). This resulted in 423-3 (8.5 g, 83.35%) as a white solid.

### 4. Synthesis of 423-4

To a solution of 423-3 (2 g, 6.985 mm ol, 1 equiv) in MeOH (40 mL) was added Pd/C (200 mg, 10%) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. The crude product was used in the next step directly without further purification. This resulted in 423-4 (1.6 g, 80.42%) as a yellow oil.

### 5. Synthesis of 423-5

To a stirred solution of 423-4 (500 mg 1,950 mmol, 1 equiv) and 1-2 (558.41 mg, 1.950 mmol, 1 equiv) in DCE (25.00 mL) was added STAB (826.75 mg, 3.900 mmol, 2 equiv) at room temperature. The resulting mixture was stirred overnight at room temperature. The reaction was quenched with NaHCO₃ (aq.) (80 mL) at room temperature. The aqueous layer was extracted with DCM (3×50 mL). The resulting mixture was concentrated under vacuum. The residue was purified by Prep-TLC (DCM/MeOH 20:1) to afford 423-5 (860 mg, 77.02%) as a yellow oil.

### 6. Synthesis of 423-6

To a stirred solution of 423-5 (860 mg, 1.633 mmol, 1 equiv) and Pyridine (1.29 g, 16.330 mmol, 10 equiv) in DCM (10 mL) was added Triphosgene (193.82 mg, 0.653 mmol, 0.4 equiv) at room temperature. The resulting mixture was stirred for 10 min at room temperature. The reaction was quenched with NaHCO₃ (aq.) (80 mL) at room temperature. The aqueous layer was extracted with DCM (3×30 mL). The resulting mixture was concentrated under vacuum. The residue was purified by Prep-TLC (DCM/MeOH 20:1) to afford 423-6 (420 mg, 46.07%) as a yellow oil.

### 7. Synthesis of 423-0

The 423-6 (400 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 30% B to 30% B in 26 min; Wave Length: 220/254 nm; RT1 (min): 10.04; RT2 (min): 19.42; the second peak is product) to afford 423 (103.1 mg, 25.54%) as a yellow solid.

LC-MS-423: (ES, m/z): [M+H]⁺ 553. H-NMR-423: (400 MHz, DMSO-d6, δ ppm): 0.81-0.83 (m, 4H), 1.03-1.07 (m, 3H), 1.57-1.62 (m, 1H), 1.62-1.68 (m, 5H), 1.68-1.98 (m, 5H), 2.01-2.08 (m, 1H), 2.74-2.76 (m, 2H), 3.20-3.24 (m, 3H), 3.83-3.89 (m, 2H), 4.24-4.27 (d, 1H), 7.01 (s, 1H), 7.21-7.23 (d, 1H), 7.29 (s, 1H), 7.41-7.45 (m, 1H), 7.65-7.70 (m, 1H), 7.70 (s, 1H), 8.42 (s, 1H).

### Example 412. Synthesis of Compound 424

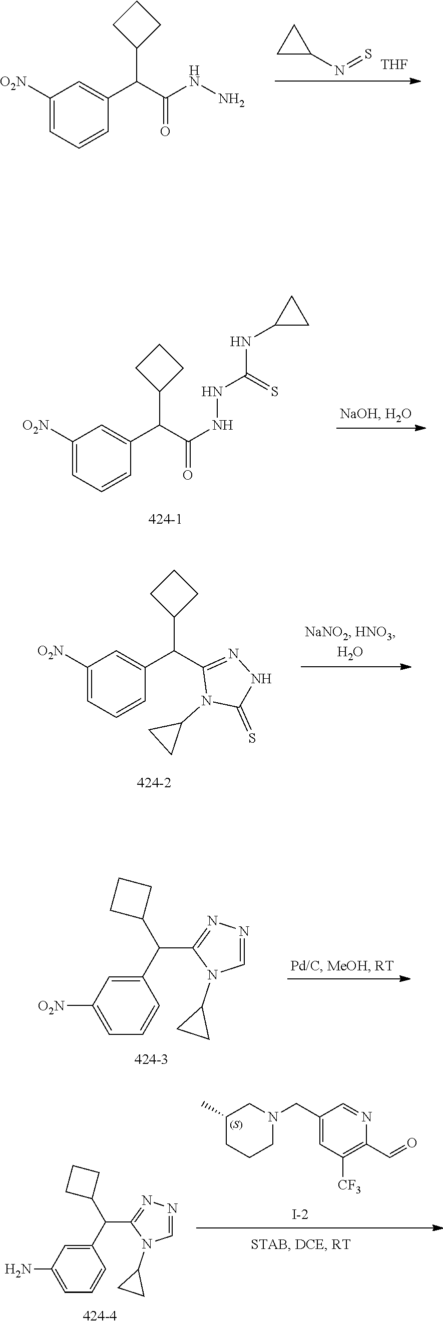

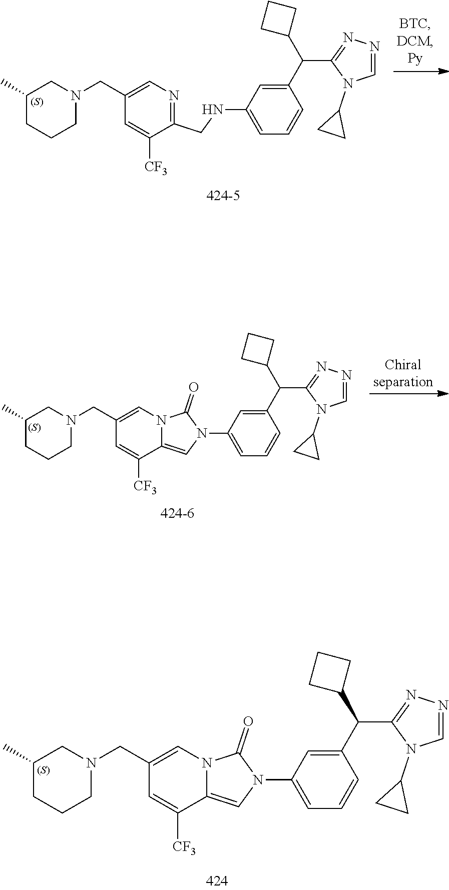

### 2. Synthesis of 424-1

To a stirred mixture of 2-cyclobutyl-2-(3-nitrophenyl)acetohydrazide (5 g, 20.059 mmol, 1 equiv) in tetrahydrofuran (50 mL) was added isothiocyanatocyclopropane (5.97 g, 60.177 mmol, 3 equiv) at room temperature. The resulting mixture was stirred overnight at room temperature. The reaction was quenched with water (50 mL) at room temperature. The aqueous layer was extracted with CH₂Cl₂ (4×50 mL). The resulting mixture was concentrated under vacuum. This resulted in 424-1 (6 g, 85.85%) as a white solid.

### 2. Synthesis of 424-2

To a stirred mixture of NaOH (6.89 g, 172.210 mmol, 10 equiv) in H₂O (172 mL) was added 424-1 (6 g, 17.221 mmol, 1 equiv) at room temperature. The resulting mixture was stirred overnight at 50° C. The mixture was neutralized to pH 7 with HCl (1M). The aqueous layer was extracted with Cl₂Cl₂ (5×50 mL). The resulting mixture was concentrated under vacuum. This resulted in 424-2 (5.2 g, 91.39%) as a yellow solid.

### 3. Synthesis of 424-3

To a stirred mixture of 424-2 (5 g, 15.133 mmol, 1 equiv) and NaNO₂ (10.44 g, 151.330 mmol, 10 equiv) in H2O (50 ml) was added HNO₃ (150 mL, 151.330 mmol, 10 equiv, 1M) dropwise at room temperature. The resulting mixture was stirred for 8 h at room temperature. The mixture was neutralized to pH 7 with NaOH (aq.). The aqueous layer was extracted with CH₂Cl₂ (5×50 mL). The resulting mixture was concentrated under vacuum. This resulted in 424-3 (3 g, 66.45%) as a yellow solid.

### 4. Synthesis of 424-4

To a solution of 424-3 (3.2 g, 10.726 mmol, 1 equiv) in 100 mL MeOH was added Pd/C (10%, 0.11 g) under nitrogen atmosphere. The mixture was hydrogenated at room temperature for overnight under hydrogen atmosphere using a hydrogen balloon, filtered through a Celite pad and concentrated under reduced pressure. This resulted in 424-4 (1.5 g, 52.11%) as a yellow solid.

### 5. Synthesis of 424-5

To a stirred mixture of 424-4 (500 mg, 1.864 mmol, 1.2 equiv) and I-2 (640.10 mg, 2.236 mmol, 1.2 equiv) in DCE (5 mL) was added STAB (1184.62 mg, 5.589 mmol, 3 equiv) at room temperature. The resulting mixture was stirred overnight at room temperature. The reaction was quenched with saturated NaHCO₃ (aq.) (80 mL) at room temperature. The aqueous layer was extracted with CH₂Cl₂ (3×30 mL). The resulting mixture was concentrated under vacuum. The residue was purified by Prep-TLC (CH₂Cl₂/MeOH 20:1) to afford 424-5 (600 mg, 59.78%) as a white solid.

### 6. Synthesis of 424-6

To a stirred mixture of 242-5 (600 mg, 1.114 mmol, 1 equiv) and Pyridine (528.64 mg 6.684 mmol, 6 equiv) in DCM (15 mL) was added Triphosgene (132.22 mg, 0.446 mmol, 0.4 equiv) at room temperature. The resulting mixture was stirred for 10 min at room temperature. The reaction was quenched with saturated NaHCO₃ (aq.) (80 mL) at room temperature. The aqueous layer was extracted with DCM (3×40 mL). The residue was purified by Prep-TLC (CH₂Cl₂/MeOH 15:1) to afford 424-6 (380 mg, 60.42%) as a yellow solid.

### 7. Synthesis of 424

424-6 (300 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 18 min; Wave Length: 220/254 nm; RT1 (min): 8.14; RT2 (min): 13.09; the second peak is product) to afford 424 (143.2 mg, 47.73%) as a yellow solid.

LC-MS-424: (ES, m/z): [M+H]⁺ 558. H-NMR-424: (400 MHz, DMSO-d₆, δ ppm): 0.58-0.59 (m, 1H), 0.81-0.87 (m, 5H), 0.94-0.92 (m, 2H), 1.57-1.59 (m, 1H), 1.61-1.79 (m, 4H), 1.80-1.88 (m, 6H), 2.10-2.21 (m, 1H), 2.72-2.76 (m, 2H), 2.97-3.00 (m, 1H), 3.24-3.33 (m, 3H), 4.34 (d, 1H), 7.00 (s, 1H), 7.20-7.22 (d, 1H), 7.31 (s, 1H), 7.42-7.46 (m, 1H), 7.65-7.68 (m, 2H), 7.78 (s, 1H), 8.35 (s, 1H).

### Example 413. Synthesis of Compound 425

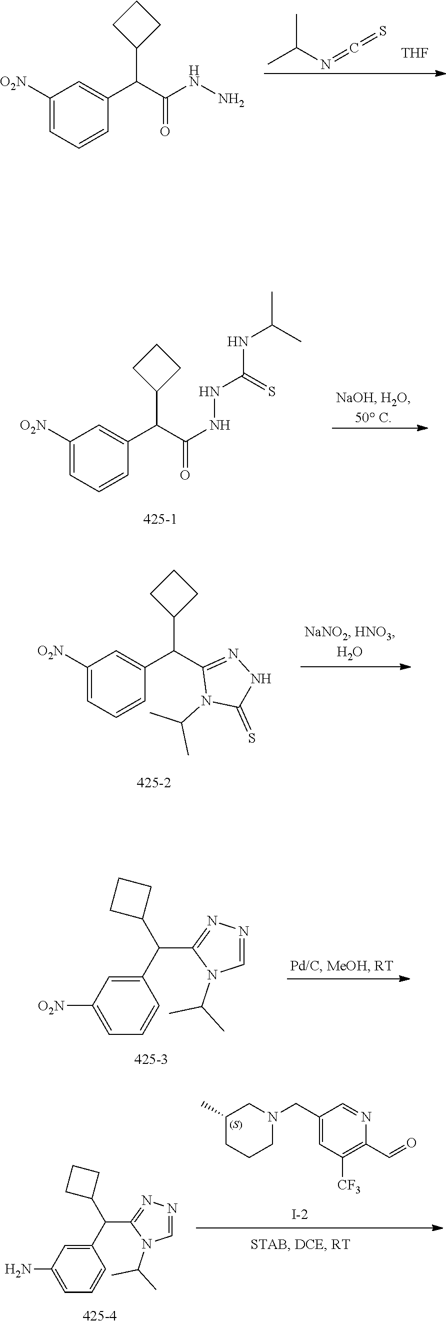

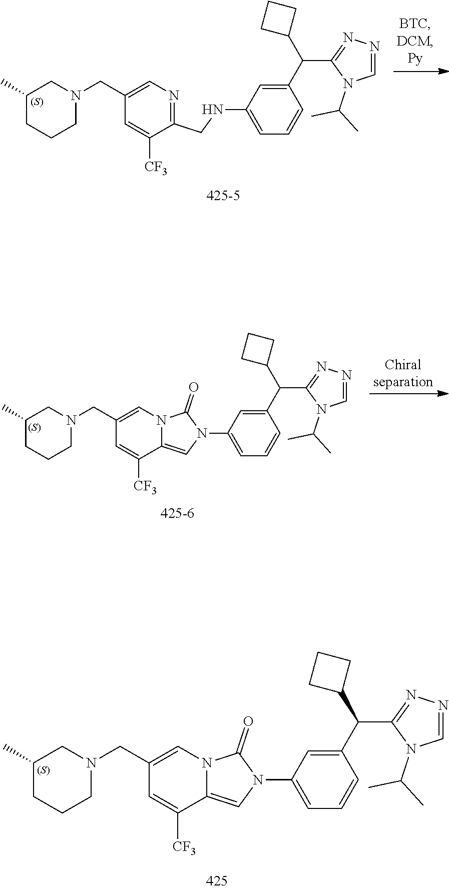

### 1. Synthesis of 425-1

A solution of 2-cyclobutyl-2-(3-nitrophenyl)acetohydrazide (5 g& 20.059 mmol, 1 equiv) and 2-isothiocyanatopropane (3.04 g, 30.089 mmol, 1.5 equiv) in tetrahydrofuran (50 mL) was stirred for 5 h at room temperature. The resulting mixture was diluted with water (80 mL). The precipitated solids were collected by filtration and washed with water (3×20 ml). This resulted in 425-1 (6 g, 76.82%) as a yellow solid.

### 2. Synthesis of 425-2

A solution of 425-1 (6 g, 17.121 mmol, 1 equiv) and NaOH (6.85 g, 171,210 mmol, 10 equiv) in H₂O (170 mL) was stirred overnight at 50° C. The mixture was neutralized to pH 6 with 1M HC (aq.). The precipitated solids were collected by filtration and washed with water (3×20 mL). This resulted in 425-2 (4 g, 63.25%) as a yellow solid,

### 3. Synthesis of 425-3

To a stirred solution of 425-2 (4 g, 12.033 mmol, 1 equiv) and NaNO2 (8.30 g, 120.330 mmol, 10 equiv) in H2O (40 mL) was added HNO₃ (120 mL, 120.33 mmol, 10 equiv, 1M) dropwise at room temperature. The mixture was neutralized to pH 7 with saturated NaHCO₃ (aq.). The aqueous layer was extracted with EtOAc (3×100 mL). The resulting mixture was concentrated under reduced pressure. The residue was purified by silica gel column chromatography, eluted with PE/EA (50:1) to afford 425-3 (3.2 g, 79.68%) as a yellow solid.

### 4. Synthesis of 425-4

To a solution of 425-3 (3.8 g, 12.651 mmol, 1 equiv) in MeOH (100 mL) was added Pd/C (380 mg, 10%) under nitrogen atmosphere in a 250 mL round-bottom flask. The mixture was hydrogenated at room temperature for 3 h under hydrogen atmosphere using a hydrogen balloon, filtered through a Celite pad and concentrated under reduced pressure. This resulted in 425-4 (3.2 g, 84.19%) as a light yellow solid.

### 5. Synthesis of 425-5

A solution of 425-4 (500 mg, 1.849 mmol, 1 equiv) and I-2 (635.32 mg, 2.219 mmol, 1.2 equiv) and STAB (783.86 mg, 3.698 mmol, 2 equiv) in DCE (5 mL) was stirred for 4 h at room temperature under air atmosphere. The mixture was quenched with saturated NaHCO₃ (aq.) (50 mL). The aqueous layer was extracted with CH2Cl₂ (3×30 mL). The resulting mixture was concentrated under reduced pressure. The residue was purified by Prep-TLC (CH2Cl₂/MeOH 20:1) to afford 425-5 (600 mg, 57.01%) as a yellow solid.

### 6. Synthesis of 425-6

To a stirred solution of 425-5 (580 mg, 1.073 mmol, 1 equiv) and Pyridine (509.11 mg, 6.438 mmol, 6 equiv) in DCM (10 mL) was added Triphosgene (117.77 mg, 0.397 mmol, 0.37 equiv) at room temperature. The result mixture was stirred for 10 min at room temperature. The mixture was quenched with saturated NaHCO₃ (aq.) (50 mL). The aqueous layer was extracted with CH₂Cl₂ (3×30 mL). The resulting mixture was concentrated under reduced pressure. The residue was purified by Prep-TLC (CH₂Cl₂/MeOH 12:1) to afford 425-6 (350 mg, 54.70%) as a yellow solid.

### 7. Synthesis of 425-7

The 425-6 (350 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 12.5 min; Wave Length: 220/254 nm; RT1 (min): 7.13; RT2 (min): 9.28; the second peak is product) to afford 425 (115.0 mg, 32.85%) as a yellow solid.

LC-MS-425 (ES, m/z): [M+H]⁺ 567

H-NMR-425 (400 MHz, DMSO-d6, δ ppm): 0.84-0.92 (m, 4H), 0.92-0.94 (d, 3H), 1.38-1.40 (d, 311), 1.59-1.89 (m, 11H), 2.08-2.12 (m, 1H), 2.68-2.75 (m, 2H), 3.21-3.33 (m, 3H), 4.27-4.36 (m, 2H), 7.01 (s, 1H), 7.19-7.21 (d, 1H), 7.28 (s, 1H), 7.41-7.45 (m, 1H), 7.65-7.69 (m, 2H), 7.75 (s, 1H), 8.59 (s, 1H).

### Example 414. Synthesis of Compound 426

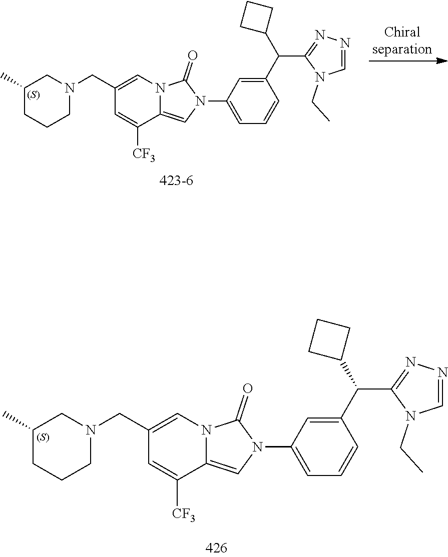

### 1. Synthesis of 426

The 423-6 (420 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 30% B to 30% B in 26 min; Wave Length: 220/254 nm; RT (min): 10.04; RT2 (min): 19.42; the first peak is product) to afford 426 (1373 mg, 34.25%) as a yellow solid.

LC-MS-426: (ES, m/z): [M+H]⁺ 553. H-NMR-426: (400 MHz, DMSO-d6, δ ppm): 0.81-0.83 (m, 4H), 1.03-1.07 (m, 3H), 1.57-1.62 (m, 1H), 1.62-1.68 (m, 5H), 1.68-1.98 (m, 5H), 2.01-2.08 (m, 1H), 2.74-2.76 (m, 2H), 3.20-3.24 (m, 3H), 3.83-3.89 (m, 2H), 4.24-4.27 (d, 1H), 7.01 (s, 1H), 7.21-7.23 (d, 1H), 7.29 (s, 1H), 7.41-7.45 (m, 1H), 7.65-7.70 (m, 1H), 7.70 (s, 1H), 8.42 (s, 1H).

### Example 415. Synthesis of Compound 427

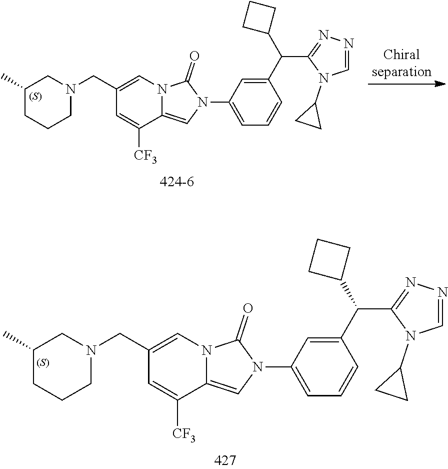

### 1. Synthesis of 427

424-6 (300 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/in; Gradient: 40% B to 40% B in 18 min; Wave Length: 220/254 nm; RT1 (min): 8.14; RT2 (min): 13.09; the first peak is product) to afford 427 (151.0 mg, 50.33%) as a yellow solid.

LC-MS-427: (ES, m/z): [M+H]⁺ 565. H-NMR-427: (400 MHz, DMSO-d₆, δ ppm): 0.58-0.59 (m, 1H), 0.81-0.87 (m, 5H), 0.94-0.92 (m, 2H), 1.57-1.59 (m, 1H), 1.61-1.79 (m, 4H), 1.80-1.88 (m, 6H), 2.10-2.21 (m, 1H), 2.72-2.76 (m, 2H), 2.97-3.00 (m, 1H), 3.24-3.33 (m, 3H), 4.34 (d, 1H), 7.00 (s, 1H), 7.20-7.22 (d, 1H), 7.31 (s, 1H), 7.42-7.46 (m, 1H), 7.65-7.68 (m, 2H), 7.78 (s, 1H), 8.35 (s, 1H).

### Example 416 Synthesis of Compound 428

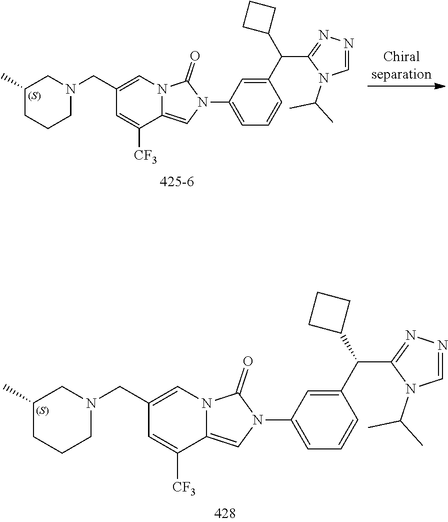

### 1. Synthesis of 428

The 425-6 (350 mg) was purified by Prep-Chiral-HPLC with the following conditions (Column: CHIRALPAK IC, 2*25 cm, 5 μm; Mobile Phase A: Hex (0.5% 2M NH₃-MeOH)-, Mobile Phase B: EtOH:DCM=1:1; Flow rate: 20 mL/min; Gradient: 40% B to 40% B in 12.5 min; Wave Length: 220/254 nm; RT1 (min): 7.13; RT2 (min): 9.28; the first peak is product) to afford 428 (99.5 mg, 28.24%) as a yellow solid.

LC-MS-428: (ES, m/z): [M+H]⁺ 567. H-NMR-428: (400 MHz, DMSO-d6, δ ppm): 0.84-0.92 (m, 4H), 0.92-0.94 (d, 3H), 1.38-1.40 (d, 3H), 1.41-1.89 (m, 11H), 2.08-2.12 (m, 1H), 2.68-2.75 (m, 2H), 3.21-3.33 (m, 3H), 4.27-4.36 (m, 2H), 7.01 (s, 1H), 7.19-7.21 (d, 1H), 7.28 (s, 1H), 7.41-7.45 (m, 1H), 7.65-7.69 (m, 2H), 7.75 (s, 1H), 8.59 (s, 1H).

### Example 417. Synthesis of Compound 429

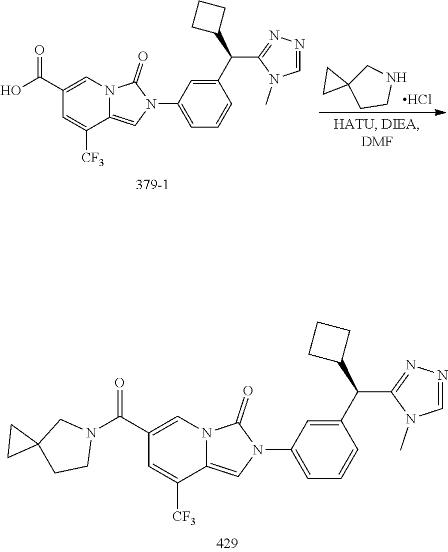

### 1. Synthesis of 429

To a stirred solution of 379-1 (200 mg, 0.424 mmol, 1.00 equiv), HATU (241.96 mg, 0.636 mmol, 1.5 equiv), DIEA (164.49 mg, 1.272 mmol, 3 equiv) and DMF (2 mL) was added 5-azaspiro [2.4] heptane hydrochloride (68.02 mg, 0.509 mmol, 1.2 equiv) at room temperature. The resulting mixture was stirred for overnight at room temperature. The reaction was quenched by the addition of water (4 mL) at room temperature. The aqueous layer was extracted with EtOAc (1×4 mL). The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH4HCO₃), Mobile Phase B: ACN; Flow rate: 60 mL/min; Gradient: 33% B to 52% B in 8 min; Wave Length: 254 nm; RT1 (min): 7.57) to afford 429 (81.2 mg, 34.49%) as a yellow solid.

LC-MS-429: (ES, m/z): [M+H]⁺ 551. H-NMR-429: 1H NMR (400 MHz, DMSO-d6 ppm) δ 0.65 (s, 4H), δ 1.70-1.83 (m, 7H), δ 2.07-2.09 (d, 1H), δ 3.21-3.22 (m, 1H), δ 3.37 (s, 1H), δ 3.42 (s, 3H), δ 3.56-3.60 (d, 2H), δ 3.79 (s, 1H), δ 4.25-4.28 (d, 1H), δ 7.15-7.22 (m, 2H), δ 7.41-7.47 (m, 2H), δ 7.66-7.68 (d, 1H), δ 7.76 (s, 1H), δ 8.00-8.02 (d, 1H), δ 8.33 (s, 1H).

### Example 418. Synthesis of Compound 430

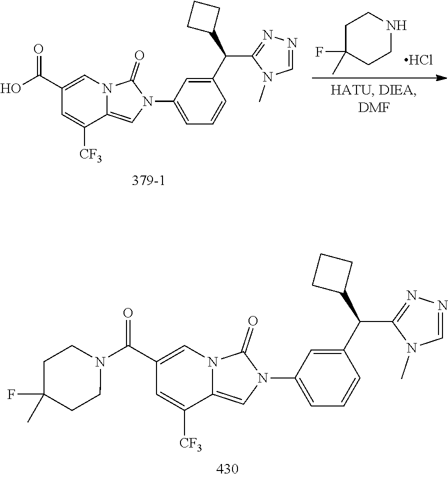

### 1. Synthesis of 430

To a stirred solution of 379-1 (200 mg, 0.424 mmol, 1.00 equiv), HATU (241.96 mg, 0.636 mmol, 15 equiv), DIEA (164.49 mg, 1.272 mmol, 3 equiv) and DMF (2 mL) was added 4-fluoro-4-methylpiperidine hydrochloride (78.21 mg, 0,509 mmol, 1.2 equiv). The resulting mixture was stirred for overnight at room temperature under. The reaction was quenched by the addition of water (4 mL) at room temperature. The aqueous layer was extracted with EtOAc (1×4 mL). The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 53% B in 8 min; Wave Length: 220 nm; RT1 (min): 7.23) to afford 430 (1282 mg, 52.01%) as a yellow solid.

LC-MS-430: (ES, m/z): [M+H]⁺ 571. H-NMR-430: 1H NMR (400 MHz, DMSO-d6 ppm) δ 1.31-1.41 (d, 3H), δ 1.70-1.80 (m, 9H), δ 2.08-2.10 (d, 1H), δ 3.19-3.25 (m, 3H), δ 3.43 (s, 3H), δ 3.90 (s, 2H), δ 4.25-4.28 (d, 1H), δ 7.06 (s, 1H), δ 7.21-7.23 (d, 1H), δ 7.41-7.47 (m, 2H), δ 7.67-7.69 (d, 1H), δ 7.75 (s, 1H), δ 7.88 (s, 1H), δ 8.35 (s, 1H).

### Example 419. Synthesis of Compound 431

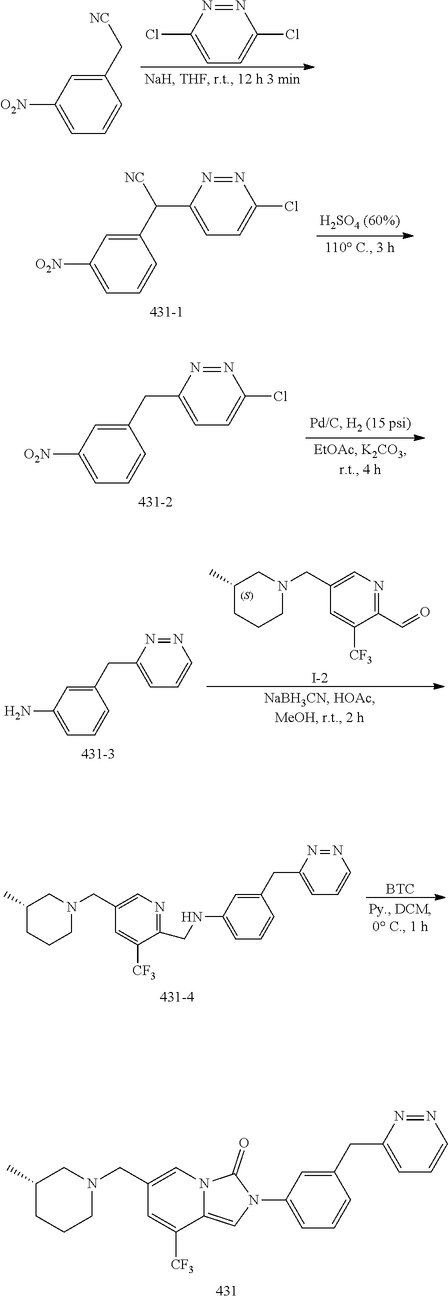

### 1. Synthesis of 431-1

To a solution of 2-(3-nitrophenyl)acetonitrile (5 g, 30.8 mmol, 1 equiv) in THF (150 mL) was added NaH (1.85 g, 46.2 mmol, 60% purity, 1.5 equiv) and stirred for 3 min until no bubbles are formed. And then a solution of 3,6-dichloropyridazine (4.59 g, 30.8 mmol, 1 equiv) in THF (50 mL) was added to reaction mixture dropwise. The result mixture was stirred at 25° C. for 12 hr under nitrogen atmosphere. The reaction mixture was diluted with water (300 mL) and extracted with EtOAc (300 mL×3). The combined organic layers were dried over anhydrous Na2SO4, filtered and concentrated under reduced pressure to give a residue. The residue was purified by flash silica gel chromatography eluted with PE/EtOAc (1:1) to afford 431-1 (2.6 g, 31%) as a brown solid.

¹H NMR-431-1: (400 MHz, CHLOROFORM-d) δ 8.30 (t, J=2.0 Hz, 1H), 8.22-8.16 (m, 1H), 7.81 (d, J=8.0 Hz, 1H), 7.63-7.52 (m, 3H), 5.71 (s, 1H)

### 2. Synthesis of 431-2

A mixture of 431-1 (2 g, 7.28 mmol, 1 equiv) in H₂SO₄ (20 mL, 60% purity) was degassed and purged with nitrogen for 3 times, and then the mixture was stirred at 110° C. for 3 h under nitrogen atmosphere. The reaction mixture was poured into ice, and then adjusted pH to 9 by ammonia and extracted with DCM (30 mL×3). The combined organic layers were dried over anhydrous Na₂SO₄, filtered and concentrated under reduced pressure to give a residue. The residue was purified by flash silica gel chromatography eluted with PE/EtOAc (1:2) to afford 431-2 (1.57 g, 86%) as a pink solid.

¹H NMR-431-2: (400 MHz, CHLOROFORM-d) δ 8.10-8.05 (m, 2H), 7.57 (d, J=8.0 Hz, 1H), 7.48-7.37 (m, 2H), 7.23 (d, J=8.8 Hz, 1H), 4.38 (s, 2H)

### 3. Synthesis of 431-3

To a solution of 431-2 (1 g, 4.01 mmol, 1 equiv) in EtOAc (10 mL) was added K₂CO₃ (553 mg, 4.01 mmol, 1 equiv) and Pd/C (I g, 1.6 mmol, 10% purity). The mixture was stirred at 25° C. for 3 h under hydrogen atmosphere (15 psi). The reaction mixture was filtered and the filtrate was concentrated under reduced pressure to afford 431-3 (620 mg, crude) as a white solid.

### 4. Synthesis of 431-4

To a solution of 431-3 (620 rug, 3.35 mmol, 1 equiv) and I-2 (958 mg, 3.35 mmol, 1 equiv) in MeOH (8 mL) was added HOAc (574 uL, 10.0 mmol, 3 equiv). The mixture was stirred at 25° C. for 1 h under nitrogen atmosphere. And then NaBH3CN (420 mg, 6.69 mmol, 2 equiv) was added to reaction mixture. The mixture was stirred at 25° C. for 1 h under nitrogen atmosphere. The reaction mixture was concentrated under reduced pressure to give a residue. The residue was adjusted pH to 8 with saturated NaHCO₃ and extracted with EtOAc (20 ml×3). The combined organic layers were dried over anhydrous Na₂SO4, filtered and concentrated under reduced pressure to give a residue. The residue was purified by flash silica gel chromatography eluted with DCM/MeOH (10:1) to afford 431-4 (890 mg, 58%) as a yellow solid.

### 5. Synthesis of 431

To a solution of 431-4 (660 mg, 1.45 mmol, 1 equiv) in DCM (5 ml) was added Py. (701 uL, 8.69 mmol, 6 equiv). And bis(trichloromethyl) carbonate (860 mg, 2.9 mmol, 2 equiv) was added to reaction solution at 0° C. The mixture was stirred at 25° C. for 1 h under nitrogen atmosphere. The reaction mixture was diluted with saturated.NaHCO₃ (10 mL), extracted with DCM (10 mu×3). The combined organic layers were dried over anhydrous Na₂SO₄, filtered and concentrated in vacuum. The residue was purified by flash silica gel chromatography elated with DCM/MeOH (10:1) to give crude product. The crude product (80 rug) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 10% B to 40% B in 8 min; Wave Length: 220 nm; RT (min): 8.4) afford 431 (30 mg, 4.3%) as a yellow solid.

MS-431: (ES, m/z): [M+H]⁺ 482.2. ¹H-NMR-431: (400 MHz, METHANOL-d₄) δ 9.11-9.06 (m, 1H), 8.48-8.30 (m, 1H), 7.86 (s, 1H), 7.77-7.70 (m, 2H), 7.70-7.63 (m, 2H), 7.51 (t, J=7.6 Hz, 1H), 7.36 (d, J=7.6 Hz, 1H), 7.24 (s, 1H), 7.13 (s, 1H), 4.44 (s, 2H), 3.79 (s, 2H), 3.28-3.14 (m, 2H), 2.53-2.43 (m, 1H), 2.25-2.15 (m, 1H), 1.78 (s, 3H), 1.76-1.65 (m, 1H), 1.17-1.04 (m, 1H), 1.00-0.92 (m, 3H).

### Example 420. Synthesis of Compound 432

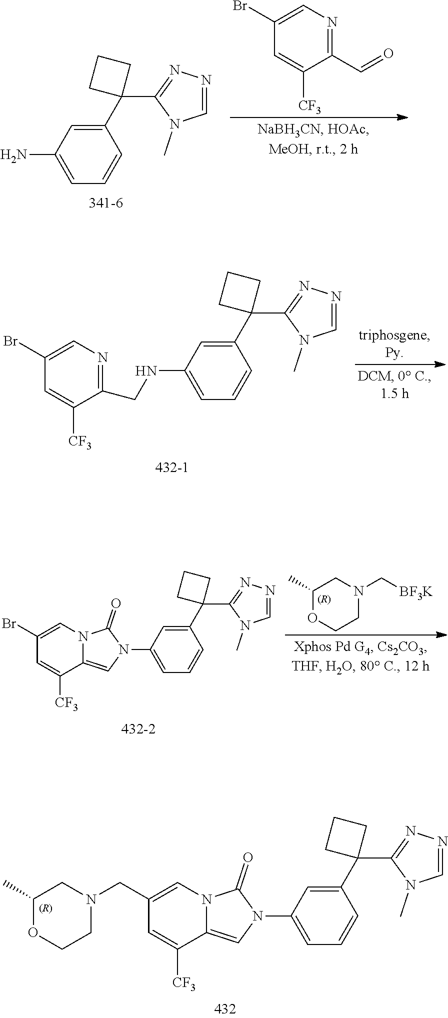

### 1. Synthesis of 432-1

To a solution of 341-6 (1.5 g, 6.57 mmol, 1 equiv) and I-2 (1.5 g, 5.91 mmol, 0.9 equiv) in MeOH (20 mL) was added AcOH (1.13 mL, 19.7 mmol, 3 equiv), the mixture was stirred at 25° C. for 1 h under nitrogen atmosphere, then NaBH₃CN (825 mg, 13.1 mmol, 2 equiv) was added, the result mixture was stirred at 25° C. for 1 5 h under nitrogen atmosphere. The reaction mixture was concentrated in vacuum. The residue was diluted with Sat. NaHCO₃ (25 mL), extracted with EtOAc (25 mL×3) The combined organic layers were dried over anhydrous Na2SO4, filtered and concentrated in vacuum. The residue was purified by silica gel column chromatography eluted with CH2Cl2/MeOH (10:1) to afford 432-1 (1.83 g, 60%) as a yellow solid.

¹H-NMR-432-1: (400 MHz, METHANOL-d₄) δ=8.84 (d, J=1.5 Hz, 1H), 8.35-8.27 (m, 2H), 7.14 (t, J=7.9 Hz, 1H), 6.64-6.57 (m, 2H), 6.50 (d, J=1.8 Hz, 1H), 4.52 (s, 2H), 3.19 (s, 3H), 2.93-2.85 (m, 2H), 2.73-2.65 (m, 2H), 2.13-2.04 (m, 2H)

### 2. Synthesis of 432-2

To a solution of 432-1 (0.5 g, 1.07 mmol, 1 equiv) in DCM (8 mL) was added pyridine (519 uL, 6.43 mmol, 6 equiv) and bis(trichloromethyl) carbonate (318 mg, 1.07 mmol, 1 equiv) at 0° C., the result mixture was stirred at 25° C. for 1 h under nitrogen atmosphere. The reaction mixture was diluted with Sat.NaHCO₃ (10 mL), extracted with DCM (8 mL×3). The combined organic layers were dried over anhydrous Na₂SO₄, filtered and concentrated in vacuum. The residue was purified by silica gel column chromatography eluted with CH2Cl2/MeOH (10:1) to afford 432-2 (500 mg, 94.7%) as a yellow solid.

¹H-NMR-432-2: (400 MHz, METHANOL-d₄) δ 8.37 (s, 1H), 7.97 (s, 1H), 7.78 (s, 1H), 7.63-7.55 (m, 2H), 7.32 (d, J=7.6 Hz, 1H), 7.26 (s, 1H), 7.08 (s, 1H), 3.34 (s, 3H), 3.07-2.99 (m, 2H), 2.87-2.78 (m, 2H), 2.20-2.09 (m, 2H)

### 3. Synthesis of 432

To a solution of a BF₃ salt (0.25 g, 1.13 mmol, 2 equiv) and 432-2 (278 mg, 565 umol, 1 equiv) in THF (4 mL) and H2O (1 mL) was added Cs₂CO₃ (552 mg, 17 mmol, 3 equiv) and dicyclohexyl-[2-(2,4,6-triisopropylphenyl)phenyl]phosphane;methanesulfonate;[2-[2-(methylamino)phenyl]phenyl]palladium(1+) (48.6 mg, 56.5 mmol, 0.1 equiv), the mixture was stirred at 80° C. for 12 h under nitrogen atmosphere. The reaction mixture was diluted with water (10 ml), extracted with EtOAc (10 mL×3). The combined organic layers were dried over anhydrous Na₂SO₄, filtered and concentrated in vacuum. The residue was purified by silica gel column chromatography eluted with CH2Cl2/MeOH (10:1) to give crude product. The crude product (300 mg) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 15% B to 50% B in 8 min; Wave Length: 220 nm; RT1 (min): 8.0) to afford 432 (101 mg, 33.5%) as a yellow solid.

MS-432: (ES, m/z): [M+H]⁺ 527.2. ¹H-NMR-432: (400 MHz, METHANOL-d₄) δ 8.36 (s, 1H), 7.76 (t, J=2.0 Hz, 1H), 7.69 (s, 1H), 7.63-7.50 (m, 2H), 7.34-7.27 (m, 1H), 7.15 (s, 1H), 7.09 (s, 1H), 3.90-3.82 (m, 1H), 3.71-3.60 (m, 2H), 3.37 (s, 2H), 3.33 (s, 3H), 3.07-2.95 (m, 2H), 2.88-2.73 (m, 4H), 2.27-2.07 (m, 3H), 1.95-1.87 (m, 1H), 1.13 (d, J=6.4 Hz, 3H).

### Example 421. Synthesis of Compound 433

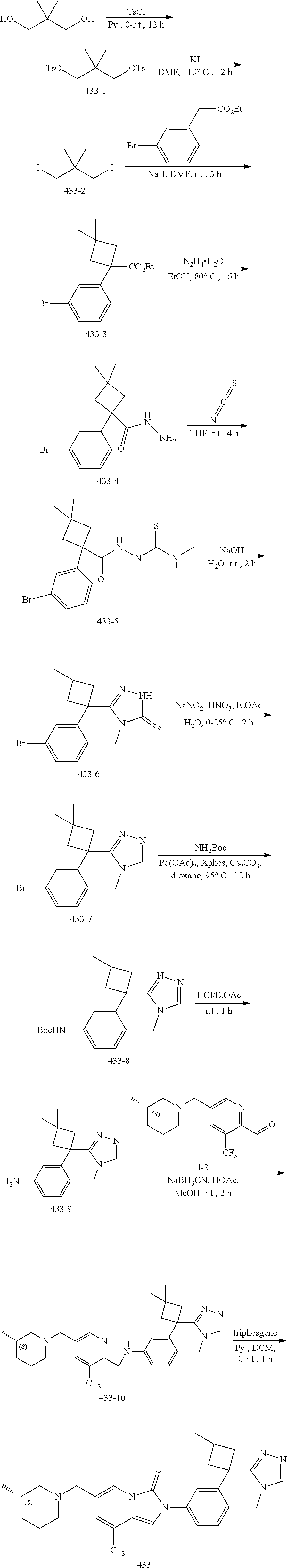

### 1. Synthesis of 433-1

To a solution of 2,2-dimethylpropane-1,3-diol (20 g, 192 mmol, 1 equiv) in Py. (120 mL) was added a solution of 4-methylbenzenesulfonyl chloride (109 g, 576 mmol, 3 equiv) in Py. (120 mL) at 0° C. The mixture was stirred at 25° C. for 12 h. The mixture was quenched with ice and water (500 mL), then the mixture was filtered. The filter cake was concentrated under reduced pressure to afford 433-1 (79 g, 99%) as a white solid,

H-NMR-433-1: (400 MHz, DMSO-d6) δ 7.74 (d, J=8.4 Hz, 4H), 7.48 (d, J=8.0 Hz, 4H), 3.73 (s, 4H), 2.44 (s, 6H), 0.79 (s, 6H)

### 2. Synthesis of 433-2

A solution of 433-1 (27 g, 65.4 mmol, 1 equiv) and KI (65.19 g, 392.71 mmol, 6 equiv) in DMF (270 mL) was stirred at 110° C. for 12 hr. The reaction mixture was extracted with water (450 ml) and EtOAc (150 mL×3). The combined organic layers were dried over anhydrous Na2SO4, filtered and the filtrate was concentrated under reduced pressure to give a residue. The residue was purified by flash silica gel chromatography eluted with EA/PE=0:1 to afford 433-2 (16.5 g, 78%) as a colorless oil.

### 3. Synthesis of 433-3

To a solution of 433-2 (12 g, 37.0 mmol, 0.9 equiv) and ethyl 2-(3-bromophenyl)acetate (10 g, 41.1 mmol, 1 equiv) in DMF (120 mL) was added NaH (3.29 g, 82.3 mmol, 60% purity, 2 equiv), the mixture was stirred at 25° C. for 3 hr. The reaction mixture was diluted with water (150 mL), extracted with EtOAc (90 mL×3). The combined organic layers were dried over anhydrous Na2SO4, filtered and concentrated in vacuum. The residue was purified by flash silica gel chromatography eluted with EA/PE=1:9 to afford 433-3 (7 g, 55%) as a colorless oil.

H-NMR-433-3: (400 MHz, CHLOROFORM-d) δ 7.37 (t, J=2.0 Hz, 1H), 7.29-7.25 (m, 1H), 7.20-7.14 (m, 1H), 7.12-7.07 (m, 1H), 4.05-3.98 (m, 2H), 2.78-2.57 (m, 2H), 2.37-2.14 (m, 2H), 1.10 (t, J=7.1 Hz, 3H), 1.07 (s, 3H), 0.94 (s, 3H)

### 4. Synthesis of 433-4

To a solution of ethyl 433-3 (6.9 g, 22.1 mmol, 1 equiv) in EtOH (35 mL) was added N2H4·H2O (27.5 mL, 554 mmol, 98% purity, 25 equiv), the mixture was stirred at 80° C. for 16 hr. The reaction mixture was concentrated in vacuum to remove EtOH. The residue was diluted with water (50 mL) and brine (10 mL), extracted with DCM (50 mL×3). The combined organic layers were dried over anhydrous Na2SO4, filtered and concentrated in vacuum to afford 433-4 (6.5 g, crude) as a colorless oil.

H-NMR-433-4: (400 MHz, DMSO-d6) δ 9.12-9.07 (m, 1H), 7.46-7.45 (m, 1H), 7.37-7.32 (m, 1H), 7.28-7.20 (m, 2H), 4.14 (d, J=4.4 Hz, 2H), 2.62 (s, 1H), 2.58 (s, 1H), 2.16 (d, J=12.8 Hz, 2H), 1.02 (s, 3H), 0.90 (s, 3H)

### 5. Synthesis of 433-5

To a solution of 433-4 (6.5 g, 21.8 mmol, 1 equiv) in THF (65 mL) was added methylimino(thioxo)methane (2.99 mL, 43.7 mmol, 2 equiv), the mixture was stirred at 25° C. for 4 hr. The reaction mixture was diluted with water (70 mL) and EtOAc (50 mL). The mixture was filtered and the filter cake was concentrated in vacuum to afford 433-5 (7.5 g, 93%) as a white solid.

H-NMR-433-5: (400 MHz, DMSO-d6) δ 9.75 (s, 1H), 9.21 (s, 1H), 7.58 (s, 1H), 7.46-7.37 (m, 2H), 7.35-7.27 (m, 1H), 7.24-6.99 (m, 1H), 2.89-2.75 (m, 5H), 2.25 (d, J=12.4 Hz, 2H), 1.13 (s, 3H), 0.94 (s, 3H)

### 6. Synthesis of 433-6

To a solution of NaOH (6.48 g, 162.03 mmol, 8 equiv) in 1120 (65 mL) was added 433-5 (7.5 g, 20.2 mmol, 1 equiv), the mixture was stirred at 25° C. for 2 hr. The reaction mixture was diluted with water (70 mL), adjusted pH to 3 by 1N HCl. The mixture was filtered and the filter cake was concentrated in vacuum to afford 433-6 (7 g, 98%) a: a white solid.

H-NMR-433-6: (400 MHz, DMSO-d6) δ 13.75 (s, 1H), 7.51-7.46 (m, 1H), 7.44 (d, J=1.6 Hz, 1H), 7.38-7.30 (m, 2H), 3.02 (s, 3H), 2.78 (d, J=12.8 Hz, 2H), 2.54 (d, J=12.8 Hz, 2H), 1.08 (d, J=6.8 Hz, 6H)

### 7. Synthesis of 433-7

HNO₃ (13.1 mL, 198 mmol, 68% purity, 10 equiv) was added to H2O (184 mL) to afford the diluted HNO3 solution (1M, 197 ml). To a solution of 433-6 (7 g, 19.87 mmol, 1 equiv) and NaNO2 (13.7 g, 198 mmol, 10 equiv) in H2O (70 mL) and EtOAc (7.0 mL, 71.5 mmol, 3.6 equiv) was added diluted HNO3 solution (1 M, 197 mL) dropwise at 0° C. Then the mixture was stirred at 25° C. for 2 hr. The reaction mixture was quenched by the addition of Sat NaHCO₃ (600 mL). The aqueous layer was extracted with EtOAc (150 mL×3). The combined organic layers were dried over anhydrous Na2SO4, filtered and concentrated in vacuum to afford 433-7 (5 g, 78%) as a yellow solid.

H-NMR-433-7: (400 MHz, METHANOL-d4) δ 8.32 (s, 1H), 7.47-7.43 (m, 1H), 7.43-7.39 (m, 1H), 7.36-7.32 (m, 1H), 7.32-7.26 (m, 1H), 3.34-3.32 (m, 3H), 2.96-88 (m, 2H), 2.72-2.62 (m, 2H), 1.15 (s, 3H), 1.11 (s, 3H)

### 8. Synthesis of 433-8

To a solution of 433-7 (1 g, 3.12 mmol, 1 equiv) and NH2Boc (439 mg, 3.75 mmol 1.2 equiv) in dioxane (15 mL) was added Cs2CO3 (1.42 g, 4.37 mmol, 4 equiv), Pd(OAc)2 (35.1 mg, 156 umol, 0.05 equiv) and XPhos (149 mg, 312 umol, 0.1 equiv), the mixture was stirred at 95° C. for 12 h under nitrogen atmosphere. The reaction mixture was diluted with water (20 mL), extracted with EtOAc (15 mL*3). The combined organic layers were dried over anhydrous Na2SO4, filtered and concentrated in vacuum. The residue was purified by flash silica gel chromatography eluted with CH2Cl2/MeOH=10:1 to afford 433-8 (800 mg, 72%) as a yellow oil.

### 9. Synthesis of 433-9

To a solution of 433-8 (800 mg, 2.24 mmol, 1 equiv) in EtOAc (10 mL) was added HCl/EtOAc (10 mL). The mixture was stirred at 25° C. for 1 hr. The reaction mixture was adjusted pH to 8 with saturated NaHCO₃ and extracted with EtOAc (20 mL×3). The combined organic layers were dried over anhydrous Na2SO4, filtered and concentrated under reduced pressure to afford 433-9 (410 mg, crude) as a white solid.

### 10. Synthesis of 433-10

To a solution of 433-9 (360 mg, 1.40 mmol, 1 equiv) and I-2 (402 mg, 1.4 mmol, 1 equiv) in MeOH (5 mL) was added HOAc (241 uL, 4.21 mmol, 3 equiv). The mixture was stirred at 25° C. for 1 hr. Add then NaBH3CN (176 mg, 2.81 mmol, 2 equiv) was added to reaction mixture. The result mixture was stirred at 25° C. for 1 h under nitrogen atmosphere. The reaction mixture was adjusted pH to 8 with saturated NaHCO₃ and extracted with EtOAc (20 mL×3). The combined organic layers were dried over anhydrous Na2SO4, filtered and concentrated under reduced pressure to give a residue. The residue was purified by flash silica gel chromatography eluted with CH2Cl2/MeOH=10:1 to afford 433-10 (730 mg, 87%) as a yellow solid

### 11. Synthesis of 433

To a solution of 433-10 (330 mg, 626 umol, 1 equiv) in DCM (5 mL) was added Py (303 uL 3.76 mmol, 6 equiv). And bis(trichloromethyl) carbonate (186 mg, 626 umol, 1 equiv) was added to reaction solution at 0° C. The mixture was stirred at 25° C. for 1 h under nitrogen atmosphere. The reaction mixture was diluted with saturated.NaHCO3 (10 mL), extracted with CH2Cl2 (10 mL×3). The combined organic layers were dried over anhydrous Na2SO4, filtered and concentrated in vacuum. The residue was purified by flash silica gel chromatography eluted with CH2Cl2/MeOH=10:1 to give crude product. The crude product (340 mg) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 15% B to 55% B in 8 min; Wave Length: 220 nm; RT1 (min): 6.5) to afford 433 (151.7 mg, 44%) as a yellow solid.

MS-433: (ES, m/z): [M+H]+ 553.3

1H NMR-433: (400 MHz, METHANOL-d4) δ 8.42 (s, 1H), 8.34 (s, 1H), 7.79 (s, 2H), 7.62-7.51 (m, 2H), 7.39 (d, J=7.2 Hz, 1H), 7.23-7.07 (m, 2H), 4.60 (s, 1H), 3.65 (s, 2H), 3.41 (s, 3H), 3.14-3.08 (m, 1H), 2.99 (d, J=12.8 Hz, 2H), 2.78 (d, J=12.8 Hz, 2H), 2.39-2.26 (m, 1H), 2.10-1.97 (m, 1H), 1.82 (d, J=12.0 Hz, 3H), 1.73-1.64 (m, 1H), 1.20 (s, 3H), 1.15 (s, 3H), 1.09-1.01 (m, 1H), 0.96 (d, J=6.4 Hz, 3H).

### Example 422. Synthesis of Compound 434_P1

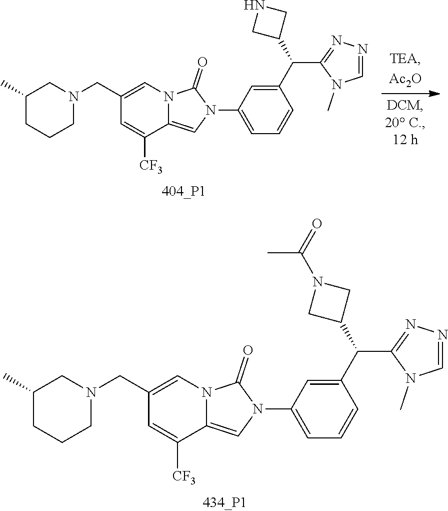 [SMILES: C[C@H]1CCCN(Cc2cc(C(F)(F)F)c3cn(-c4cccc([C@@H](c5nncn5C)C5CNC5)c4)c(=O)n3c2)C1 ; CC(=O)N1CC([C@@H](c2cccc(-n3cc4c(C(F)(F)F)cc(CN5CCC[C@H](C)C5)cn4c3=O)c2)c2nncn2C)C1]

### 1. Synthesis of 434_P1

To a solution of 404 P1 (150 mg, 277 umol, 1 equiv) in DCM (2 mL) was added TEA (116 uL, 833 umol, 3 equiv) and Ac₂O (52 uL, 556 umol, 2 equiv). The mixture was concentrated under reduced pressure (20° C.). The residue was purified by Prep-HPLC with the following conditions (column: Phenomenex Luna C18 200*40 mm*0 um; Mobile Phase A: water (FA), Mobile Phase B: ACN; Flow rate: 50 mL/min; Gradient: 5% B to 35% B in 8 min; Wave Length: 220 nm; RT1 (min): 8.2) to afford 434_P1 (50.9 mg, 29%) as a yellow solid.

MS-434_P1: (ES, m/z): [M+H]⁺ 582.2. ¹H-NMR-434_P1: (400 MHz, METHANOL-d4) δ 8.45 (s, 1H), 8.41 (d, J=2.4 Hz, 1H), 7.87 (s, 1H), 7.76-7.66 (m, 2H), 7.54 (t, J=7.6 Hz, 1H), 7.35-7.29 (m, 1H), 7.21 (s, 1H), 7.14 (s, 1H), 4.68-4.61 (m, 1H), 4.46 (t, J=8.8 Hz, 0.5H), 4.26-4.06 (m, 1.5H), 3.94-3.89 (m, 1H), 3.85 (d, J=5.2 Hz, 2H), 3.73-3.58 (m, 2H), 3.51 (s, 3H), 3.23 (d, J=11.2 Hz, 2H), 2.60-2.47 (m, 1H), 2.32-2.19 (m, 1H), 1.81-1.67 (m, 7H), 1.15-1.04 (m, 1H), 0.96 (d, J=6.4 Hz, 3H).

### Example 423. Synthesis of Compound 434_P2

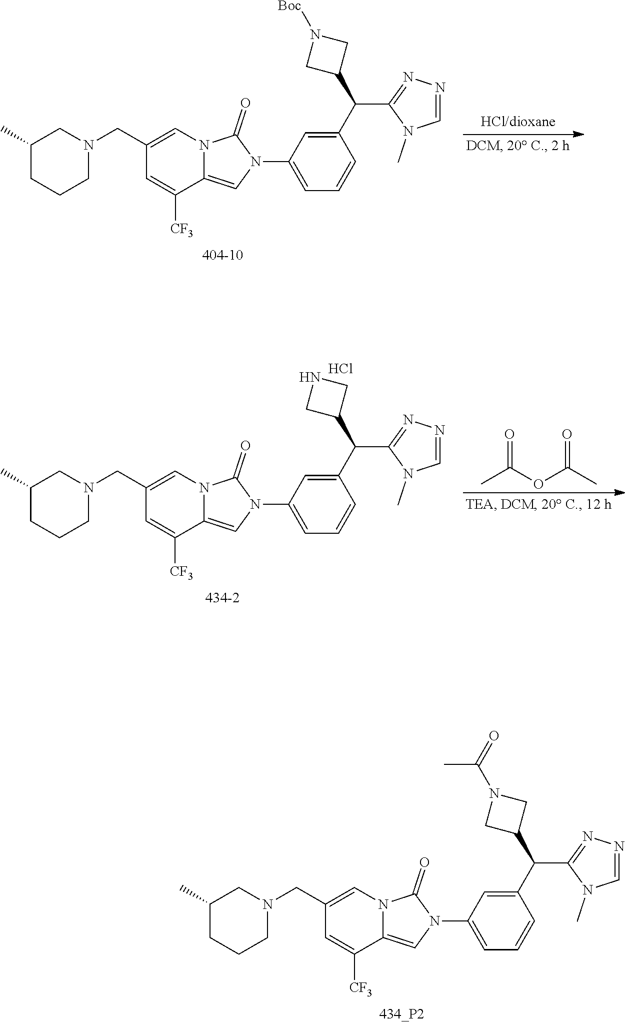

### 1. Synthesis of 434-2

To a solution of 404-10 (0.13 g, 203 umol, 1 equiv) in DCM (1 mL) was added HCl/dioxane (4 M, 305 uL, 6 equiv). The mixture was stirred at 20° C. for 2 h. The mixture was concentrated under reduced pressure to afford 434-2 (0.13 g, crude) as a yellow solid.

### 2. Synthesis of 434_P2

To a solution of 434-2 (0.1 g, 173.59 umol, 1 equiv, HCl) in DCM (2 mL) was added Ac₂O (35.44 mg, 347.19 umol, 32.52 uL, 2 equiv), TEA (52.70 mg, 520.78 umol, 72.49 uL, 3 equiv). The mixture was stirred at 20° C. for 12 h. It worked up with a small test scale (20 mg) together. The mixture was concentrated under reduced pressure to give a residue. The residue was purified by prep-HPLC (column: Phenomenex Luna C18 75*30 mm*3 um; mobile phase: [water (FA)-ACN]; B %: 15%-55%, 8 min, Wave Length: 220 nm; RT1 (min): 6.5 min) to afford 434 P2 (31 mg, 25.61% yield) as a yellow solid.

MS-434_P2: (ES, m/z): [M+H]+ 582.2. ¹H-NMR-434_P2: (400 MHz, MeOH) S 8.40 (d, J=2.8 Hz, 2H), 7.84 (s, 1H), 7.73-7.70 (m, 2H), 7.68 (t, J=1.6 Hz, 1H), 7.33-7.30 (m, 1H), 7.20 (s, 1H), 7.12 (s, 1H), 4.65-4.61 (m, 1H), 4.46 (t, J=8.4 Hz, 0.5H), 4.25-4.20 (m, 0.5H), 4.18-4.07 (m, 1H), 3.93-3.90 (m, 1H), 3.79-3.76 (m, 2H), 3.68-3.65 (m, 2H), 3.50 (s, 3H), 3.30-3.17 (m, 2H), 2.51-2.42 (m, 1H), 2.23-2.14 (m, 1H), 1.85-1.69 (m, 7H), 1.12-1.03 (m, 1H), 0.96 (d, J=6.4 Hz, 3H).

### Example 424. Synthesis of Compound 435

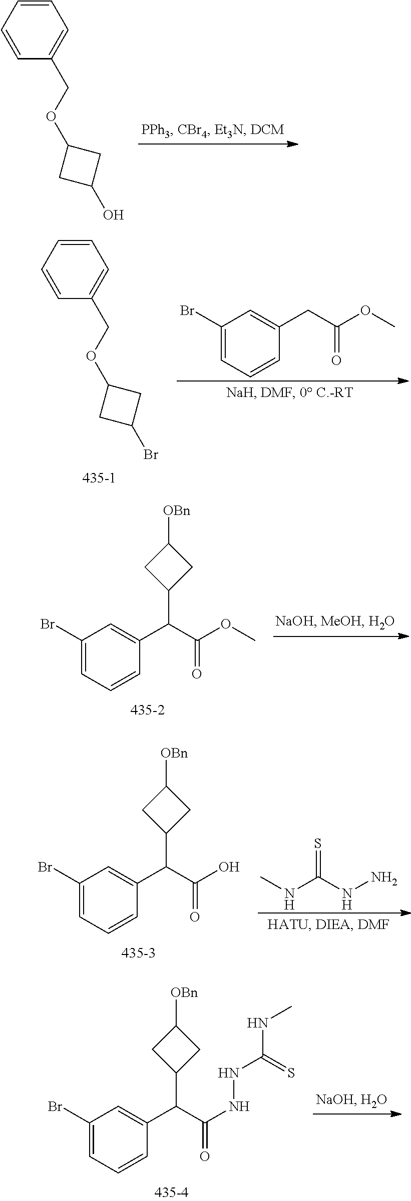

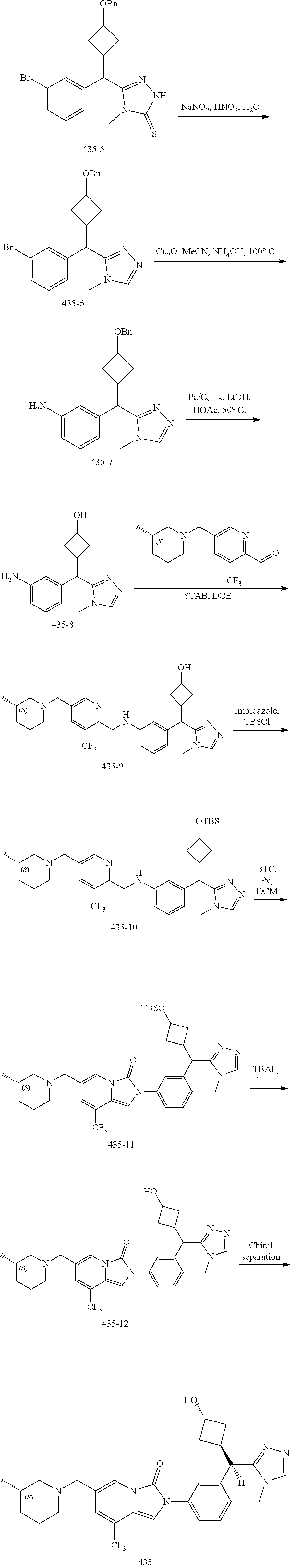

### 1. Synthesis of 435-1

Into a 2 L 3-necked round-bottom flask were added 3-(benzyloxy)cyclobutan-1-ol (29 g, 162.710 mml, 1 equiv), PPh₃ (106.69 g, 406.775 mmol, 2.5 equiv), CBr₄ (134.90 g, 406375 mmol, 2.5 equiv) and DCM (1006 mL) at 0° C. To the above mixture was added Et₃N (37.05 g, 366.098 mmol, 2.25 equiv) dropwise over 50 min at 0° C. The resulting mixture was stirred for additional overnight at room temperature. The reaction was diluted with sat. NaHCO₃ (aq.)(1000 mL). The resulting mixture was extracted with DCM (2×500 mL). The combined organic layers were concentrated under vacuum. The residue was purified by silica gel column chromatography, eluted with PE/EA (20:1) to afford 435-1 (32 g, 79.12%) as a yellow oil.

### 2. Synthesis of 435-2

Into a 1 L 3-necked round-bottom flask were added 435-1 (32.01 g, 132.744 mmol, 1.2 equiv) and DMF (320 mL) at 0° C. To the above mixture was added NaH (5.31 g, 132.744 mmol, 1.2 equiv, 60%) in portions at 0° C. The resulting mixture was stirred for additional 0.5 h at 0° C. To the above mixture was added methyl 2-(3-bromophenyl)acetate (25.34 g, 110.620 mmol, 1.00 equiv) at 0° C. The resulting mixture was stirred for additional overnight at room temperature. The reaction was quenched with sat. NH4Cl (aq. 960 ml) at room temperature. The resulting mixture was extracted with EtOAc (3×320 mL). The combined organic layers were washed with brine (320 mL). The combined organic layers was concentrated under vacuum. The residue was purified by silica gel column chromatography, eluted with PE/EA (10:1) to afford 435-2 (40 g, 83.60%) as a yellow oil.

### 3. Synthesis of 435-3

Into a 1 L 3-necked round-bottom flask were added 435-2 (40 g, 102.751 mmol, 1 equiv) MeOH (720 mL) and H2O (360 mL) and NaOH (16.44 g, 411.004 mmol, 4 equiv) at room temperature. The resulting mixture was stirred for overnight at room temperature. The MeOH was removed under vacuum. The mixture was acidified to pH 5 with HCl (aq.)(I M). The aqueous layer was extracted with EtOAc (3×400 mL). The resulting mixture was concentrated under vacuum. The residue was purified by silica gel column chromatography, eluted with PE/EA (5:1) to afford 435-3 (32.5 g, 81.76%) as a white solid.

### 4. Synthesis of 435-4

Into a 250 mL 3-necked round-bottom flask were added 435-3 (10 g, 26.648 mmol, 1 equiv), DMF (100 mL), DMA. (6.89 g, 53.296 mmol, 2 equiv) and HATU (12.16 g, 31.978 mmol, 1.2 equiv) at room temperature. The resulting mixture was stirred for 2 h at room temperature. To the above mixture was added 4-methyl-3-thiosemicarbazide (3.64 g, 34.642 mmol, 1.3 equiv) at room temperature. The resulting mixture was stirred for additional overnight at room temperature. The reaction was quenched with water (300 mL) at room temperature. The precipitated solids were collected by filtration and washed with water (2×30 mL). This resulted in 435-4 (8.6 g, 69.79%) as a white solid.

### 5. Synthesis of 435-5

Into a 250 mL round-bottom flask were added 435-4 (8.6 g, 18.598 mmol, 1 equiv), H₂O (75 mL) and NaOH (2.98 g, 74.392 mmol, 4 equiv) at room temperature. The resulting mixture was stirred for overnight at room temperature. The mixture was acidified to pH 5 with HCl (aq.). The precipitated solids were collected by filtration and washed with water (3×20 mL). This resulted in 435-5 (8.1 g, 93.11%) as a white solid.

### 6. Synthesis of 435-6

Into a 500 mL 3-necked round-bottom flask were added 435-5 (8.1 g, 18,227 mmol, 1 equiv), NaNO₂ (12.58 g, 182,270 mmol, 10 equiv) and H21O (100 mL) at room temperature. To the above mixture was added HNO₃ (91 m L, 91.135 mmol, 5 equiv, 1M) dropwise over 0.5 h at room temperature. The resulting mixture was stirred for additional overnight at room temperature. The mixture was basified to pH 7 with sat. NaHCO₃ (aq.). The resulting mixture was extracted with EtOAc (3×100 mL). The resulting mixture was concentrated under vacuum. The residue was purified by silica gel column chromatography, eluted with PE/EA (5:1) to afford 435-6 (7 g, 88.48%) as a white solid.

### 7. Synthesis of 435-7

Into a 500 mL ammonolysis kettle were added 435-6 (7 g, 16.977 mmol, 1 equiv), Cu₂O (4.86 g, 33.954 mmol, 2 equiv), MeCN (300 mL) and NH₄OH (300 mL) at room temperature. The resulting mixture was stirred for overnight at 100° C. The resulting mixture was extracted with CH₂Cl₂/MeOH (10:1) (5×300 ml). The resulting mixture was concentrated under vacuum. The residue was purified by silica gel column chromatography, eluted with CH₂Cl₂/MeOH (10:1) to afford 435-7 (2.5 g, 38.04%) as a green oil.

### 8. Synthesis of 435-8

To a solution of 435-7 (2.14 g, 6.141 mmol, 1 equiv) in EtOH (20 mL) and HOAc (20 mL) was added Pd/C (321 mg, 10%) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. The residue was purified by Prep-TLC (CH₂Cl₂/MeOH 15:1) to afford 435-8 (500 mg, 28.36%) as a white solid.

### 9. Synthesis of 435-9

To a stirred solution of 435-8 (500 mg, 1.936 mmol, 1 equiv) and 1-2 (664.97 mg, 2,323 mmol, 1.2 equiv) in DCE (10 mL) was added STAB (820.44 mg, 3.872 mmol, 2 equiv). The resulting mixture was stirred for overnight at room temperature. The resulting mixture was diluted with water (15 mL). The resulting mixture was extracted with CH₂Cl₂ (3×10 mL). The combined organic layers were concentrated under reduced pressure. The residue was purified by Prep-TLC (CH₂Cl₂/MeOH 15:1) to afford 435-9 (750 mg, 73.30%) as a white solid.

### 10. Synthesis of 435-10

To a stirred solution of 435-9 (750 mg, 1.419 mmol, 1 equiv) and Imidazole (193.18 mg, 2.838 mmol, 2 equiv) in THF (15 mL) was added TBSCl (427.68 mg, 2.838 mmol, 2 equiv). The resulting mixture was stirred for 5 h at room temperature. The resulting mixture was diluted with water (45 mL). The resulting mixture was extracted with EtOAc (4×30 mL). The combined organic layers were concentrated under reduced pressure. The residue was purified by Prep-TLC (CH₂Cl₂/MeOH 25:1) to afford 435-10 (640 mg, 70.17%) as a white solid.

### 11. Synthesis of 435-11

To a stirred solution of 435-10 (640 mg, 0.996 mmol, 1 equiv) and Pyridine (393.72 mg, 4.980 mmol, 5 equiv) in DCM (20 mL) was added Triphosgene (118.16 mg, 0.398 mmol, 0.4 equiv) at 0° C. The resulting mixture was stirred for 2 h at 0° C. under. The reaction was quenched by the addition of sat. NaHCO₃ (aq.) (15 mL). The resulting mixture was extracted with CH2Cl/MeOH=10/1 (2×20 mL). The combined organic layers were concentrated under reduced pressure. The residue was purified by Prep-TLC (Cl2Cl2/MeOH 20:1) to afford 435-11 (507 mg, 76.14%) as a yellow solid.

### 12. Synthesis of 435-12

To a stirred solution of 435-11 (507 mg, 0.758 mmol, 1 equiv) in THF (5 mL) was added TBAF (2.27 mL, 2.274 mmol, 3 equiv, 1 M). The resulting mixture was stirred for overnight at room temperature. The resulting mixture was diluted with water (20 mL). The resulting mixture was extracted with EtOAc (3×20 mL). The combined organic layers were concentrated under reduced pressure. The residue was purified by Prep-TLC (CH₂Cl₂/MeOH 10:1). The crude product was purified by reverse flash chromatography with the following conditions: column, C18 silica gel; mobile phase, MeCN in Water (10 mmol/L NH₄CO₃), 5% to 60% gradient in 25 min; detector, UV 254 nm. This resulted in 435-12 (400 mg, 95.15%) as a yellow solid.

### 13. Synthesis of 435

435-12 (400 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 70% B to 70% B in 13 min; Wave Length: 220/254 nm; RT1 (min): 4.75; RT2 (min): 8.50; RT3 (min): 10.90; first peak was product) The crude product (140 mg) was purified by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 35% B to 35% B in 12 min; Wave Length: 220/254 nm; RT1 (min): 8.04; RT2 (min): 10.09; first peak was desired product) to afford 435 (61.6 mg, 15.40%) as a yellow solid.

LCMS-435: (ES, m/z): [M+H]⁺ 555. NMR-435: (400 MHz, CD₃OD, δ ppm): 0.90-0.96 (m, 4H), 1.64-1.78 (m, 5H), 1.97-2.08 (m, 3H), 2.20-2.29 (m, 2H), 2.86-2.92 (m, 2H), 3.24-3.28 (m, 1H), 3.32 (s, 2H), 3.56 (s, 3H), 4.30-4.33 (d, 1H), 4.41-4.44 (t, 1H), 7.13 (s, 2H), 7.29-7.30 (d, 1H), 7.50-7.53 (t, 1H), 7.65-7.71 (m 3H), 8.37 (s, 1H).

### Example 425. Synthesis of Compound 436

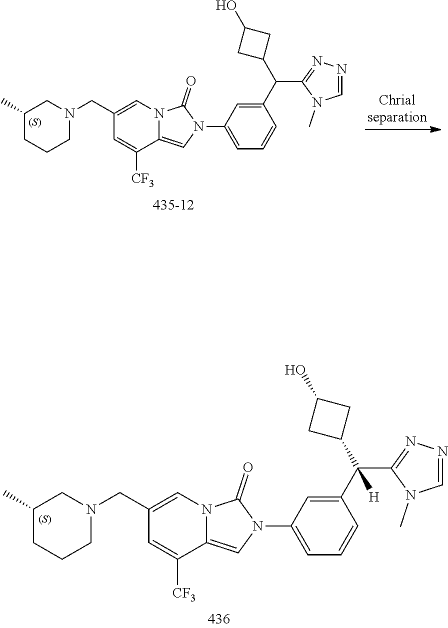

### 1. Synthesis of 436

435-12 (400 mg) was purified by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 13 min; Wave Length: 220/254 nm; RT1 (min): 4.75; RT2 (min): 8.50; RT3 (min): 10.90; the first peak was product) to afford crude product (140 mg). The crude product (140 mg) was purified by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH: DCM=1:1; Flow rate: 20 mL/min; Gradient: 35% B to 35% B in 12 min; Wave Length: 220/254 nm; RT1 (min): 8.04; RT2 (min): 10.09; the second peak was product) to afford 436 (41.9 mg, 10.47%) as a yellow solid.

LCMS-436: (ES, m/z): [M+H]⁺ 555. NMR-436: (400 MHz, CD₃OD, δ ppm): 0.90-0.96 (m, 4H), 1.31-1.85 (m, 7H), 1.96-2.01 (t, 1H), 2.22-2.25 (m, 1H), 2.57-2.61 (m, 1H), 2.74-2.85 (m, 1H), 2.87-2.91 (m, 2H), 3.24-3.26 (m, 1H), 3.28 (s, 2H), 3.54 (s, 3H), 4.11-4.15 (t, 1H), 4.22-4.25 (t, 1H), 7.12 (s, 2H), 7.25-7.27 (d, 1H), 7.49-7.53 (t, 1H), 7.66-7.72 (m 3H), 8.37 (s, 1H).

### Example 426. Synthesis of Compound 437

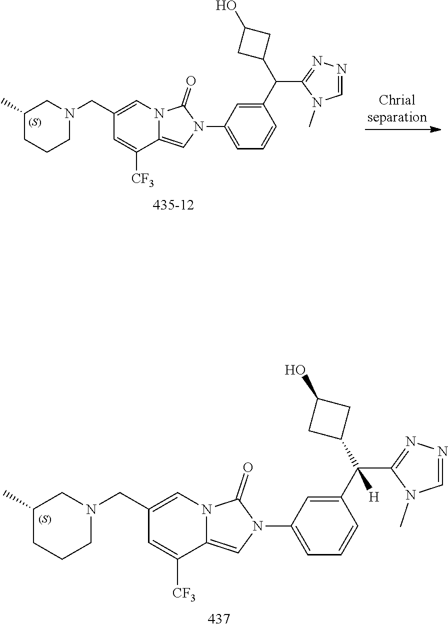

### 1. Synthesis of 437

435-12 (400 mg) was purified by Prep-Chiral-HPLC with the following conditions (Column: CHIRALPAK IG, 2*25 cm, 5 Ln; Mobile Phase A: Hex (0.5% 2M NH₃-MeOH), Mobile Phase B: EtOH:DCM=1:1; Flow rate: 20 mL/min; Gradient: 70% B to 70% B in 13 min; Wave Length: 220/254 am; RT1 (min): 4.75; RT2 (min): 8.50; RT3 (min): 10.90; second peak was desired product) to afford 437 (76.3 mg, 19.07%) as a yellow solid,

LCMS-437: (ES, m/z): [M+H]⁺ 555. NMR-437: (400 MHz, CD₃OD, δ ppm): 0.90-0.96 (m, 4H), 1.64-1.78 (m, 5H), 1.97-2.08 (m, 3H), 2.20-2.27 (m, 2H), 2.87-2.93 (m, 2H), 3.24-3.28 (m, 1H), 3.36 (s, 2H), 3.56 (s, 3H), 4.30-4.33 (d, 1H), 4.41-4.44 (t, 1H), 7.13 (s, 2H), 7.29-7.31 (d, 1H), 7.50-7.54 (t, 1H), 7.66-7.71 (m 3H), 8.37 (s, 1H).

### Example 427. Synthesis of Compound 438

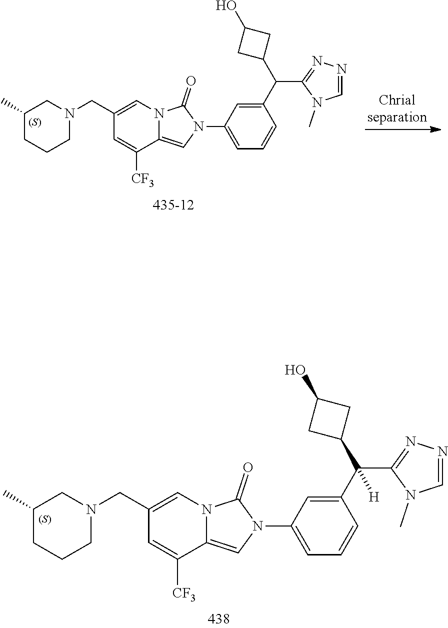

### 1. Synthesis of 438

435-12 (400 mg) was purified by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 13 min; Wave Length: 220/254 nm; RT1 (min): 4.75; RT2 (min): 8.50; RT3 (min): 10.90; the third peak was product) to afford 438 (48.8 mg 12.20%) as a yellow solid.

LCMS-438: (ES, m/z): [M+H]+ 555. NMR-438: (400 MHz, CD₃OD, δ ppm): 0.90-0.96 (m, 4H), 1.60-1.85 (m, 7H), 1.96-2.01 (t, 1H), 2.22-2.25 (m, 1H), 2.57-2.61 (m, 1H), 2.74-2.85 (m, 1H), 2.87-2.91 (m, 2H), 3.24-3.26 (m, 1H), 3.28 (s, 2H), 3.54 (s, 3H), 4.11-4.15 (t, 1H), 4.22-4.25 (t, 1H), 7.12 (s, 2H), 7.25-7.27 (d, 1H), 7.49-7.53 (t, 1H), 7.66-7.72 (m 3H), 8.37 (s, 1H).

### Example 428. Synthesis of Compound 439

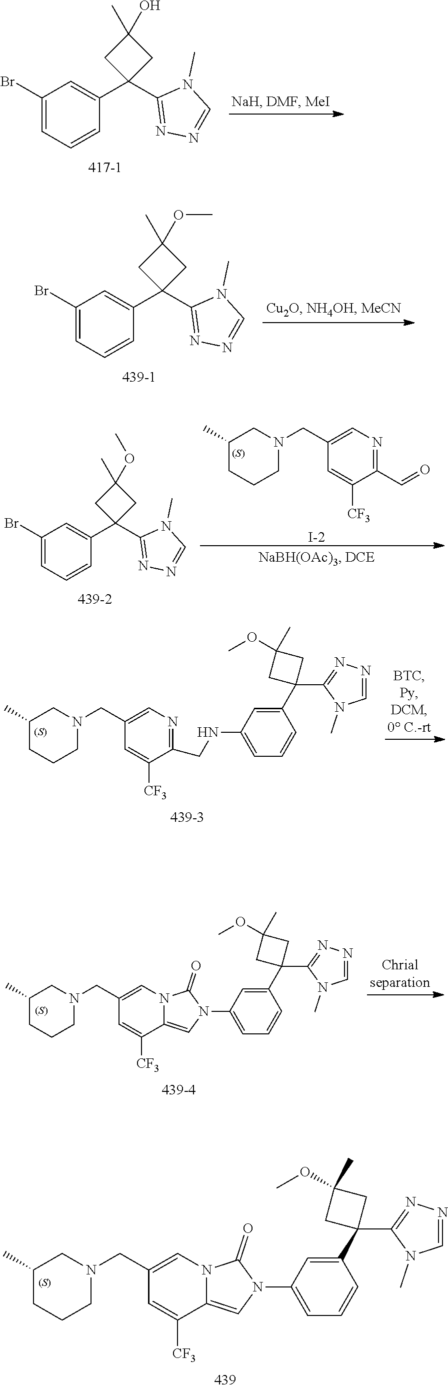

### 1. Synthesis of 439-1

To a solution of 417-1 (2 g, 6.20 mmol, 1.0 equiv) in DMF (20 mL) was added sodium hydride (60% in oil, 297 mg) at 0 degrees C. The mixture was stirred for 15 min. MeI (1.3 g, 9.31 mmol, 1.5 equiv) was added and the mixture was allowed to warm to room temperature and stirred for overnight. The reaction mixture was quenched by water and extracted with DCM (3*25 mL). The resulting mixture was concentrated under reduced pressure. The residue was purified by silica gel column chromatography, eluted with PE/EA (5:1) to afford 439-1 (800 mg, 38%) as a colorless oil.

### 2. Synthesis of 439-2

To a stirred solution of 439-1 (1.5 g, 4.69 mmol, 1.0 equiv) and Cu2O (268 mg, 1.88 mmol, 0.4 equiv) in MeCN (10 mL) was added NH₃·H₂O (10 mL) at room temperature. The resulting mixture was stirred for overnight at 100° C. The resulting mixture was diluted with water (50 mL). The resulting mixture was extracted with CH₂Cl₂ (3×30 mL). The combined organic layers were washed with water (3×30 mL), dried over anhydrous Na2SO4. After filtration, the filtrate was concentrated under reduced pressure. The residue was purified by Prep-TLC (CH₂Cl₂/MeOH=20:1) to afford 439-2 (1 g, 72%) as a colorless oil.

### 3. Synthesis of 439-3

To a stirred solution of 439-2 (1 g, 3.67 mmol, 1.0 equiv) and I-2 (1 g, 3.67 mmol, 1.0 equiv) in DCE (10 mL) was added NaBH(OAc)₃ (1.6 g, 7.34 mmol, 2.0 equiv) at room temperature. The resulting mixture was stirred for overnight at room temperature. The reaction was quenched by the addition of water (30 mL) at room temperature. The aqueous layer was extracted with CH₂Cl₂ (3×20 mL). The resulting mixture was concentrated under reduced pressure. The residue was purified by Prep-TLC (CH₂Cl₂/MeOH=10:1) to afford 439-3 (1.4 g, 70%) as an off-white solid.

### 4. Synthesis of 439-4

To a stirred solution of 439-3 (1 g, 1.84 mmol, 1.0 equiv) and pyridine (0.9 g, 11.05 mmol, 6.0 equiv) in DCM (10 mL) was added triphosgene (0.2 g, 0.64 mmol, 0.35 equiv) at room temperature. The resulting mixture was stirred for 1 h at 0° C. The reaction was quenched by the addition of sat. NaHCO₃ (aq.) (30 mL) at room temperature. The aqueous layer was extracted with CH2Cl2 (3×20 mL). The resulting mixture was concentrated under reduced pressure. The residue was purified by Prep-TLC (CH₂Cl₂/MeOH=10:1) to afford 439-4 (520 ng, 50%) as a yellow solid.

### 5. Synthesis of 439

The 439-4 (450 mg, 0.75 mmol, 1.0 equiv, 95%) was purified by chiral separation with the following conditions (Column: CHIRALPAK IG, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 40% B to 40% B in 7.5 min; Wave Length: 220/254 nm; RT1 (min): 5.12; RT2 (min): 6.49; The first peak was the product. Sample Solvent: EtOH:DCM=1:1-HPLC; Injection Volume: 0.5 mL; Number of Runs: 14) to afford 439 (48 mg, 11%) as a yellow solid.

LC-MS-439: (ES, m/z): [M+H]⁺ 569. H-NMR-439: (400 MHz, CD₃OD, δ ppm): 0.90-0.96 (m, 4H), 1.32 (s, 3H), 1.57-1.78 (m, 4H), 1.93-2.02 (m, 1H), 2.84-2.97 (m, 4H), 3.13-3.25 (m, 5H), 3.42-3.48 (m, 2H), 3.50 (s, 3H), 7.13-7.16 (d, 2H), 7.43-7.45 (d, 1H), 7.54-7.63 (m, 2H), 7.68 (s, 1H), 7.89 (s, 1H), 8.30 (s, 1H).

### Example 429. Synthesis of Compound 440

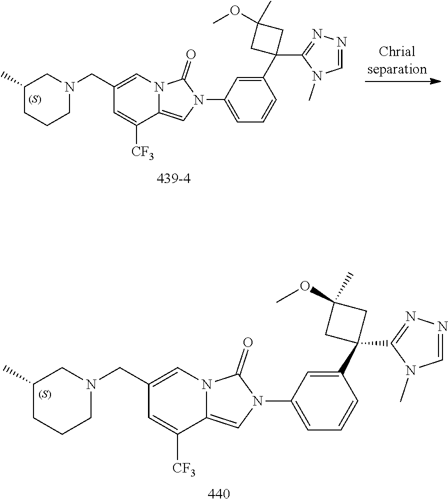

### 1. Synthesis of 440

The 439-4 (450 mg, 0.75 mmol, 1.0 equiv, 95%) was purified by chiral separation with the following conditions (Column: CHIRALPAK IG, 2*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 40% B to 40% B in 7.5 min; Wave Length: 220/254 nm; RT1 (min): 5.12; RT2 (main): 6.49; The second peak was the product. Sample Solvent: EtOH:DCM=1:1-HPLC; Injection Volume: 05 mL; Number of Runs: 14) to afford 440 (328 mg, 73%) as a yellow solid.

LC-MS-440: (ES, m/z): [M+H]⁺ 569. H-NMR-440: (400 MHz, CD₃OD, δ ppm): 0.90-0.98 (m, 4H), 1.26-1.30 (m, 3H), 1.57-1.77 (m, 4H), 1.95-2.00 (m, 1H), 2.85-2.91 (m, 2H), 2.96-2.99 (m, 2H), 3.11-3.18 (m, 2H), 3.20 (s, 3H), 3.33-3.36 (m, 2H), 3.41 (s, 3H), 7.12-7.15 (d, 2H), 7.25-7.27 (d, 1H), 7.54-7.63 (m, 2H), 7.67 (s, 1H), 7.74 (s, 1H), 8.41 (s, 1H).

### Example 430. Synthesis of Compound 441

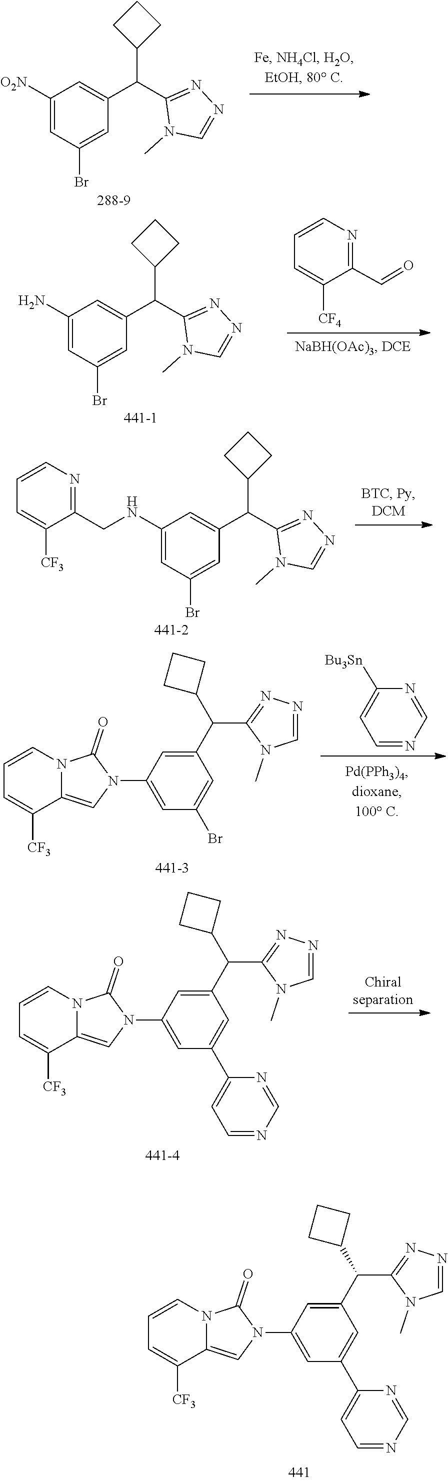

### 1. Synthesis of 441-1

To a stirred solution of 288-9 (2.75 g, 7.830 mmol, 1 equiv) and Fe (1.31 g, 23,490 mmol, 3 equiv) in EtOH (60 mL)/H₂O (20 mL) was added NH₄Cl (4.19 g, 78.300 mmol, 10 equiv) at room temperature. The resulting mixture was stirred for 3 h at 80° C. The resulting mixture was filtered, the filter cake was washed with DCM (50 ml). The filtrate was concentrated under reduced pressure. The residue was purified by silica gel column chromatography, eluted with DCM/MeOH (30:1) to afford 441-1 (2.4 g, 95.42%) as an off-white solid,

### 2. Synthesis of 441-2

To a stirred solution of 441-2 (1.4 g, 7.471 mmol, 1 equiv) and 3-(trifluoromethyl)pyridine-2-carbaldehyde (1.44 g, 8.218 mmol, 1.1 equiv) in DCE (30 mL) was added NaBH(OAc)3 (3.17 g, 14,942 mmol, 2 equiv) at room temperature. The resulting mixture was stirred for overnight at room temperature. The reaction was quenched with NaHCO₃ (aq.) (200 mL) at room temperature. The aqueous layer was extracted with DCM (3×80 mL). The combined organic layers were dried over anhydrous Na2SO4. After filtration, the filtrate was concentrated under reduced pressure. The residue was purified by trituration with MTBE (100 mL). This resulted in 441-2 (3.1 g, 82.92%) as an off-white solid.

### 3. Synthesis of 441-3

To a stirred solution of 441-2 (3.1 g, 6,454 mmol, 1 equiv) and Pyridine (5.10 g, 64,540 mmol, 10 equiv) in DCM (60 mL) were added Triphosgene (0.77 g, 2,582 mmol, 0.4 equiv) at 0° C. The resulting mixture was stirred for 10 min at room temperature. The reaction was quenched with NaHCO₃ (aq.) (200 mL) at room temperature. The aqueous layer was extracted with DCM (3×100 mL). The combined organic layers were dried over anhydrous Na2SO4. After filtration, the filtrate was concentrated under reduced pressure. The product was precipitated by the addition of MTBE. This resulted in 441-3 (3 g, 86.30%) as a yellow solid.

### 4. Synthesis of 441-4

To a stirred solution of 441-3 (400 mg, 0,790 mmol, 1 equiv) and 4-(tributylstannyl)pyrimidine (583.24 ng, 1.580 mmol, 2 equiv) in dioxane (6 ml) were added Pd(PPh₃)₄ (136.94 mg, 0.118 mmol, 0.15 equiv) at room temperature under nitrogen atmosphere. The resulting mixture was stirred overnight at 100° C. under nitrogen atmosphere. The resulting mixture was concentrated under vacuum. The residue was purified by Prep-TLC (DCM/MeOH 20:1) to afford 441-4 (300 mg, 72.12%) as a yellow solid.

### 5. Synthesis of 441

The 441-4 (300 mg) was purified by Prep-Chiral-HPLC with the following conditions (Column: CHIRALPAK IC, 2*25 cm, 5 in; Mobile Phase A: Hex (0.5% 2M NH₃-MeOH), Mobile Phase B: EtOH:DCM=1:1; Flow rate: 20 mL/min; Gradient: 70% B to 70% B in 13 min; Wave Length: 220/254 nm; RT1 (min): 9.18; RT2 (min): 11.04; the first peak is product) to afford 441 (70.8 mg, 23.08%) as a yellow solid.

LC-MS-441: (ES, m/z): [M+H]⁺ 506. H-NMR-441: (400 MHz, DMSO-d6, δ ppm): 1.73-1.84 (m, 5H), 2.11-2.13 (m, 1H), 3.28-3.30 (m, 1H), 3.48 (s, 3H), 4.40-4.43 (d, 1H), 6.29-6.32 (m, 1H), 7.11-7.13 (d, 1H), 7.52 (s, 1H), 7.79-7.81 (d, 1H), 8.04 (s, 1H), 8.09 (s, 1H), 8.16-8.17 (d, 1H), 8.35 (s, 1H), 8.41 (s, 1H), 8.82-8.93 (d, 1H), 9.29 (s, 1H).

### Example 431. Synthesis of Compound 442

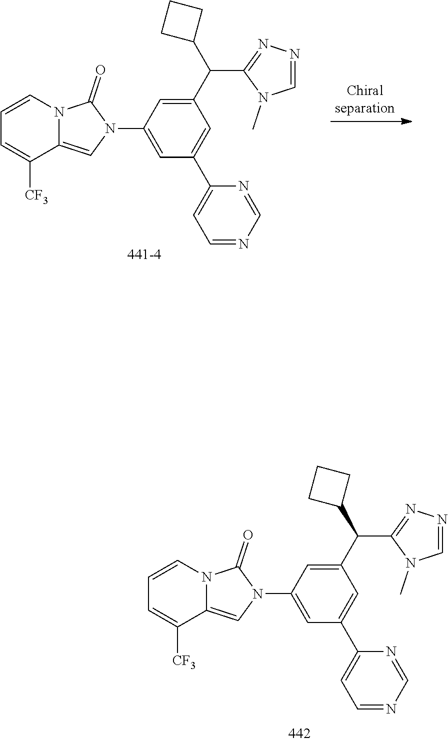 [SMILES: Cn1cnnc1C(c1cc(-c2ccncn2)cc(-n2cc3c(C(F)(F)F)cccn3c2=O)c1)C1CCC1 ; Cn1cnnc1[C@@H](c1cc(-c2ccncn2)cc(-n2cc3c(C(F)(F)F)cccn3c2=O)c1)C1CCC1]

### 1. Synthesis of 442

The 441-4 (300 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 70% B to 70% B in 13 min; Wave Length: 220/254 nm; RT1 (min): 9.18; RT2 (min): 11.04; the second peak is product) to afford 442 (66.9 mg, 2190%) as a yellow solid.

LC-MS-442: (ES, m/z): [M+H]⁺ 506. H-NMR-442: (400 MHz, DMSO-d6, δ ppm): 1.73-1.83 (m, 5H), 2.10-2.12 (m, 1H), 3.28-3.30 (m, 1H), 3.48 (s, 3H), 4.40-4.43 (d, 1H), 6.29-6.32 (m, 1H), 7.11-7.13 (d, 1H), 7.53 (s, 1H), 7.79-7.81 (d, 1H), 8.04 (s, 1H), 8.09 (s, 1H), 8.16-8.17 (d, 1H), 8.35 (s, 1H), 8.41 (s, 1H), 8.82-8.93 (d, 1H), 9.29 (s, 1H).

### Example 432. Synthesis of Compound 443

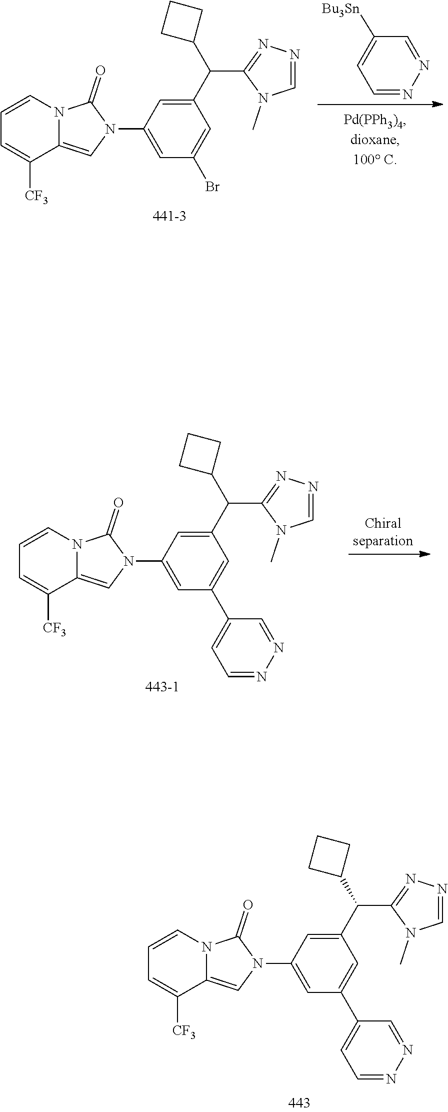

### 1. Synthesis of 443-1

To a stirred solution of 441-3 (400 mg, 0.790 mmol, 1 equiv) and 4-(tributylstannyl)pyridazine (583.24 mg, 1.580 mmol, 2 equiv) in dioxane (6 mL) was added Pd(PPh₃)₄ (136.94 mg, 0.118 mmol, 0.15 equiv) at room temperature under nitrogen atmosphere. The resulting mixture was stirred overnight at 100° C. under nitrogen atmosphere. The resulting mixture was concentrated under vacuum. The residue was purified by Prep-TLC (DCM/MeOH 15:1) to afford 443-1 (250 mg, 60.10%) as a yellow solid.

### 2. Synthesis of 443-0

The 443-1 (250 mg) was purified by Prep-Chiral-HPLC with the following conditions (Column: CHIRALPAK IC, 2*25 cm, 5 pnm; Mobile Phase A: Hex (0.5% 2M NH₃-MeOH), Mobile Phase B: EtOH:DCM=1:1; Flow rate: 20 mL/min; Gradient: 90% B to 90% B in 22 min; Wave Length: 220/254 nm; RT1 (min): 11.27; RT2 (min): 16.99; the first peak is product) to afford 443 (88.9 mg, 34.71%) as a yellow solid.

LC-MS-443: (ES, m/z): [M+H]⁺ 506. H-NMR-443: (400 MHz, DMSO-d6, δ ppm): 1.71-1.90 (m, 5H), 2.08-2.12 (m, 1H), 3.28-3.33 (m, 1H), 3.49 (s, 3H), 4.37-4.39 (d, 1H), 6.28-6.32 (m, 1H), 7.10-7.12 (d, 1H), 7.59 (s, 1H), 7.78-7.80 (d, 2H), 8.05-8.09 (d, 2H), 8.10 (s, 1H), 8.34 (s, 1H), 9.33-9.34 (d, 1H), 9.71 (s, 1H).

### Example 433. Synthesis of Compound 444

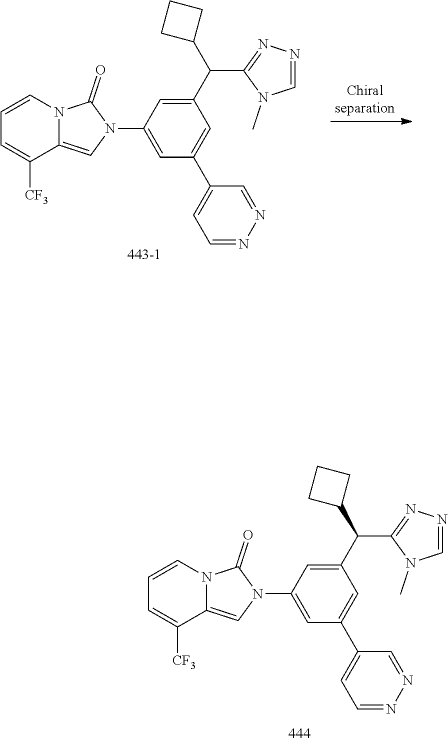 [SMILES: Cn1cnnc1C(c1cc(-c2ccnnc2)cc(-n2cc3c(C(F)(F)F)cccn3c2=O)c1)C1CCC1 ; Cn1cnnc1[C@@H](c1cc(-c2ccnnc2)cc(-n2cc3c(C(F)(F)F)cccn3c2=O)c1)C1CCC1]

### 1. Synthesis of 444

The 443-1 (250 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 90% B to 90% B in 22 min; Wave Length: 220/254 nm; RT1 (min): 11.27; RT2 (min): 16.99; the second peak is product) to afford 444 (87.0 mg, 34.35%) as a yellow solid.

LC-MS-444: (ES, m/z): [M+H]⁺ 506. H-NMR-444: (400 MHz, DMSO-d6, δ ppm): 1.73-1.89 (m, 5H), 2.08-2.12 (m, 1H), 3.28-3.33 (m, 1H), 3.49 (s, 3H), 4.37-4.39 (d, 1H), 6.28-6.32 (m, 1H), 7.10-7.12 (d, 1H), 7.59 (s, 1H), 7.78-7.80 (d, 2H), 8.05-8.10 (m, 3H), 8.35 (s, 1H), 9.33-9.34 (d, 1H), 9.71 (s, 1H).

### Example 434. Synthesis of Compound 445

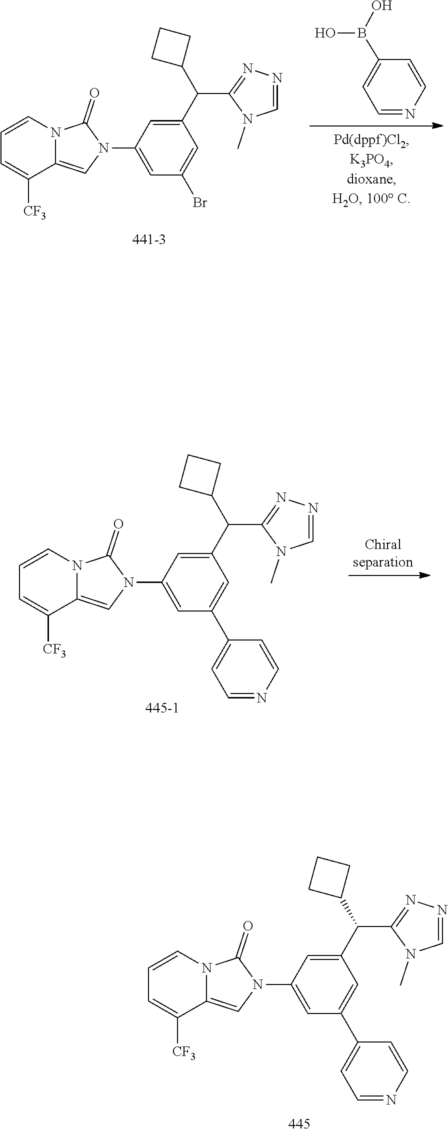 [SMILES: Cn1cnnc1C(c1cc(Br)cc(-n2cc3c(C(F)(F)F)cccn3c2=O)c1)C1CCC1 ; OB(O)c1ccncc1 ; Cn1cnnc1C(c1cc(-c2ccncc2)cc(-n2cc3c(C(F)(F)F)cccn3c2=O)c1)C1CCC1 ; Cn1cnnc1[C@H](c1cc(-c2ccncc2)cc(-n2cc3c(C(F)(F)F)cccn3c2=O)c1)C1CCC1]

### 1. Synthesis of 445-1

To a stirred solution of 441-3 (400 mg, 0.790 mmol, 1 equiv) and pyridin-4-ylboronic acid (194.21 mg, 1.580 mmol, 2 equiv) in dioxane (6 mL)/H₂O (1.5 mL) was added K3PO₄ (419.23 mg, 1.975 mmol, 2.5 equiv) and Pd(dppf)Cl2 (57.81 mg, 0.079 mmol, 0.1 equiv) at room temperature under nitrogen atmosphere. The resulting mixture was stirred for 3 h at 100° C. under nitrogen atmosphere. The resulting mixture was diluted with water (50 mL). The aqueous layer was extracted with DCM (3×30 mL). The combined organic layers were concentrated under reduced pressure. The residue was purified by Prep-TLC (DCM/MeOH 20:1) to afford 445-1 (320 mg, 77.08%) as a yellow solid.

### 2. Synthesis of 445

The 445-1 (320 mg) was purified by Prep-Chiral-HPLC with the following conditions (Column: CHIRALPAK IG, 2*25 cm, 5 n; Mobile Phase A: Hex (0.5% 2M NH₃-MeOH), Mobile Phase B: EtOH:DCM=1:1; Flow rate: 20 mL/min; Gradient 60% B to 60% B in 8.5 min; Wave Length: 220/254 nm; RT1 (min): 4.50; RT2 (min): 6.70; the first peak is product) to afford 445 (130.0 mg, 39.73%) as a yellow solid.

LC-MS-445: (ES, m/z): [M+H]⁺ 505. H-NMR-445: (400 MHz, DMSO-d6, δ ppm): 1.71-1.89 (m, 5H), 2.08-2.11 (m, 1H), 3.29-3.33 (m, 1H), 3.49 (s, 3H), 4.37-4.39 (d, 1H), 6.28-6.31 (m, 1H), 7.10-7.12 (d, 1H), 7.54 (s, 1H), 7.66 (s, 1H), 7.74-7.80 (m, 3H), 7.93 (s, 1H), 8.03 (s, 1H), 8.35 (s, 1H), 8.68-8.69 (d, 2H).

### Example 435. Synthesis of Compound 446

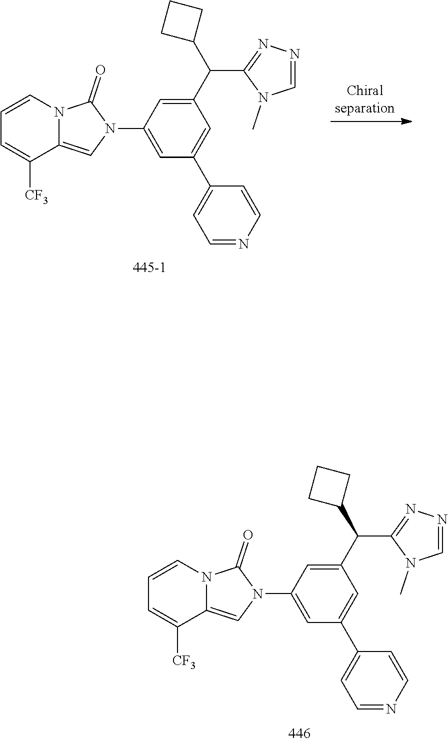 [SMILES: Cn1cnnc1C(c1cc(-c2ccncc2)cc(-n2cc3c(C(F)(F)F)cccn3c2=O)c1)C1CCC1 ; Cn1cnnc1[C@@H](c1cc(-c2ccncc2)cc(-n2cc3c(C(F)(F)F)cccn3c2=O)c1)C1CCC1]

### 1. Synthesis of 446

The 445-1 (320 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/in; Gradient: 60% B to 60% B in 8.5 min; Wave Length: 220/254 nm; RT1 (mini): 4.50; RT2 (min): 6.70; the second peak is product) to afford 446 (1293 mg, 40.04%) as a yellow solid.

LC-MS-446: (ES, m/z): [M+H]⁺ 505. H-NMR-446: (400 MHz, DMSO-d6, δ ppm): 1.71-1.89 (m, 5H), 2.08-2.13 (m, 1H), 3.29-3.33 (m, 1H), 3.49 (s, 3H), 4.37-4.39 (d, 1H), 6.28-6.31 (m, 1H), 7.10-7.12 (d, 1H), 7.54 (s, 1H), 7.66 (s, 1H), 7.74-7.80 (m, 3H), 7.93 (s, 1H), 8.02 (s, 1H), 8.34 (s, 1H), 8.67-8.69 (d, 2H).

### Example 436. Synthesis of Compound 447

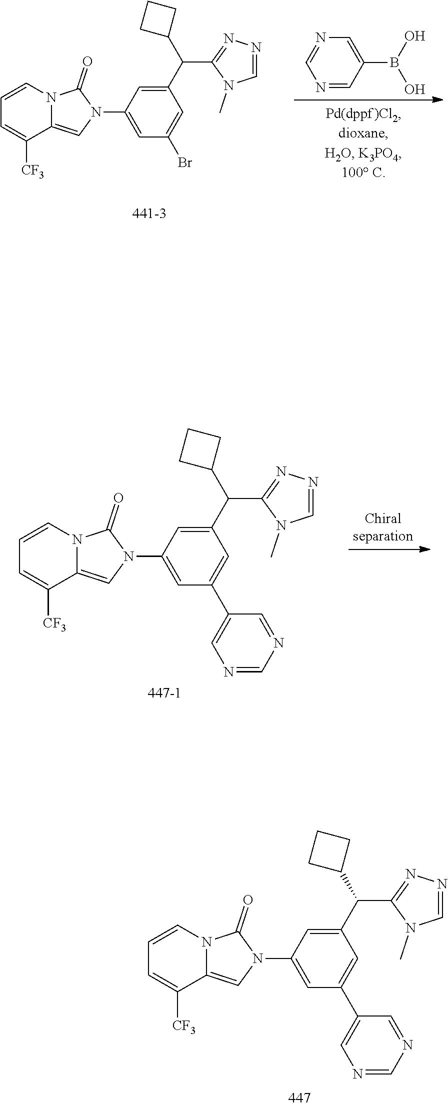 [SMILES: Cn1cnnc1C(c1cc(Br)cc(-n2cc3c(C(F)(F)F)cccn3c2=O)c1)C1CCC1 ; OB(O)c1cncnc1 ; Cn1cnnc1C(c1cc(-c2cncnc2)cc(-n2cc3c(C(F)(F)F)cccn3c2=O)c1)C1CCC1 ; Cn1cnnc1[C@H](c1cc(-c2cncnc2)cc(-n2cc3c(C(F)(F)F)cccn3c2=O)c1)C1CCC1]

### 1. Synthesis of 447-1

To a stirred mixture of 441-3 (400 mg, 0.790 mmol, 1 equiv) and pyrimidin-5-ylboronic acid (195.78 mg, 1,580 mmol, 2 equiv) in dioxane (4 mL) and H₂O (1 mL) were added K₃PO₄ (335.38 mg, 1.580 mmol, 2 equiv) and Pd(dppf)Cl₂ (57.81 mg, 0.079 mmol, 0.1 equiv) under nitrogen atmosphere. The resulting mixture was stirred for overnight at 100° C. under nitrogen atmosphere. The resulting mixture was diluted with water (30 mL). The resulting mixture was extracted with EtOAc (2×20 mL). The combined organic layers were concentrated under reduced pressure. The residue was purified by Prep-TLC (CH₂Cl₂/MeOH 20:1) to afford 447-1 (260 mg, 65.11%) as a yellow solid.

### 2. Synthesis of 447

447-1 (260 mg, 0.514 mmol, 1 equiv) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 19 min; Wave Length: 220/254 nm; RT1 (min): 11.1; RT2 (min): 14.82; the first peak was product) to afford 447 (92.3 mg, 35.50%) as a yellow solid.

LCMS-447: (ES, m/z): [M+H]⁺ 506. NMR-447: (400 MHz, DMSO, ppm): δ 1.71-1.86 (m, 5H), 2.08-2.13 (m, 1H), 3.30-3.32 (m, 1H), 3.50 (s, 3H), 4.35-4.38 (d, 1H), 6.28-6.32 (t, 1H), 7.10-7.12 (d, 1H), 7.57 (s, 1H), 7.69 (s, 1H), 7.78-7.80 (d, 1H), 8.01 (s, 1H), 8.34 (s, 1H), 9.21-9.24 (m, 3H).

### Example 437. Synthesis of Compound 448

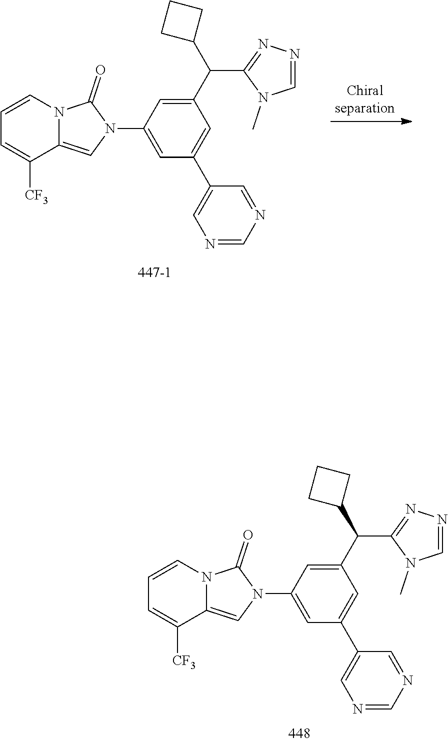 [SMILES: Cn1cnnc1C(c1cc(-c2cncnc2)cc(-n2cc3c(C(F)(F)F)cccn3c2=O)c1)C1CCC1 ; Cn1cnnc1[C@@H](c1cc(-c2cncnc2)cc(-n2cc3c(C(F)(F)F)cccn3c2=O)c1)C1CCC1]

### 1. Synthesis of 448

447-1 (260 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 19 min; Wave Length: 220/254 nm; RT1 (min): 11.1; RT2 (min): 14.82; the second peak was product) to afford 448 (94 mg, 36.15%) as a yellow solid.

LCMS-448: (ES, m/z): [M+H]⁺ 506. NMR-448: (400 MHz, DMSO, ppm): δ 1.71-1.85 (m, 5H), 2.08-2.12 (m, 1H), 3.32-3.36 (m, 1H), 3.50 (s, 3H), 4.35-4.38 (d, 1H), 6.28-6.32 (t, 1H), 7.10-7.12 (d, 1H), 7.57 (s, 1H), 7.69 (s, 1H), 7.78-7.80 (d, 1H), 8.01 (s, 1H), 8.34 (s, 1H), 9.21-9.24 (m, 3H).

### Example 438. Synthesis of Compound 449

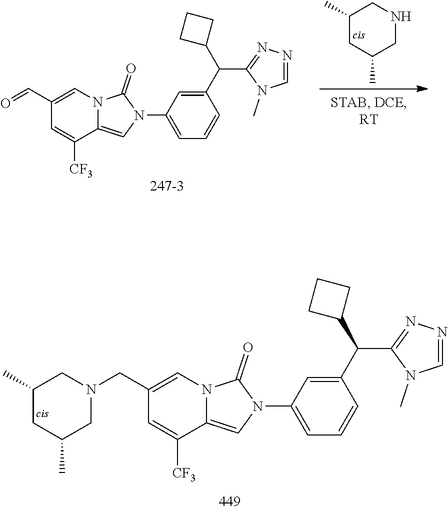

### Synthesis of 449

To a stirred solution of 247-3 (300 mg, 0.659 mmol, 1 equiv) and (3R,5S)-3,5-dimethylpiperidine (223.70 mg, 0.977 mmol, 3 equiv) in DCE (5 mL) was added STAB (279.21 mg, 1.317 mmol, 2.00 equiv) at room temperature. The resulting mixture was stirred for additional 6 h at room temperature. The reaction was quenched with saturated NH₄Cl (aq.) (50 mL) at room temperature. The aqueous layer was extracted with DCM (2×20 mL). The resulting mixture was concentrated under vacuum. The residue was purified by Prep-TLC (DCM/MeOH 20:1) to afford the crude product. The crude product was purified by Prep-HPLC with the following conditions (Column: XBridge Prep OED C18 Column, 30*150 mm, 5 μm; Mobile Phase A: Water (10 mmol/L NH₄HCO₃), Mobile Phase B: ACN; Flow rate: 60 mL/min; Gradient: 54% B to 74% B in 8 min, 74% B; Wave Length: 254 nm; RT1 (min): 7.1) to afford 449 (80.2 mg, 21.81%) as a yellow solid.

LC-MS-449: (ES, m/z): [M+H]⁺ 553. H-NMR-449: (400 MHz, DMSO-d6, δ ppm): 0.44-0.55 (m, 1H), 0.80-0.82 (d, 6H), 1.43-1.48 (m, 2H), 1.59-1.81 (m, 8H), 2.08-2.10 (m, 1H), 2.75-2.80 (m, 2H), 3.25-3.29 (m, 3H), 3.43 (s, 3H), 4.25-4.27 (d, 1H), 7.00 (s, 1H), 7.18-7.20 (d, 1H), 7.30 (s, 1H), 7.42-7.46 (m, 1H), 7.65-7.74 (m, 3H), 8.32 (s, 1H).

### Example 439. Synthesis of Compound 450

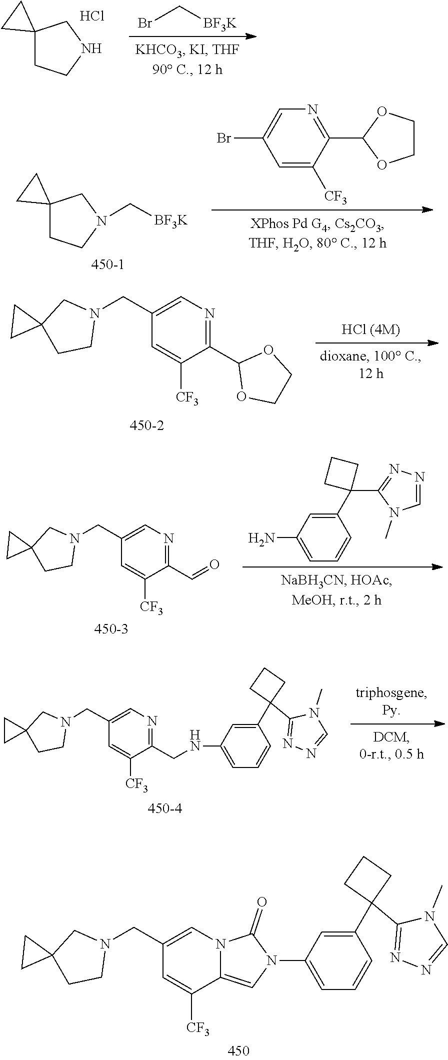

### 1. Synthesis of 450-1

A mixture of potassium;bromomethyl(trifluoro)boranuide (826.67 mg, 4.12 mmol, 1.1 equiv), 5-azaspiro[2.4]heptane (0.5 g, 3.74 mmol, 1 equiv, HCl salt), KHCO₃ (749 mg, 7.48 mmol, 2 equiv) and KI (62.1 mg, 374 umol, 0.1 equiv) in THF (8 mL) was stirred at 90° C. for 12 h under nitrogen atmosphere. The reaction mixture was concentrated in vacuum. The residue was triturated with acetone (15 ml) at 40° C. for 1 hr. Then the mixture was filtered and the filtrate was concentrated in vacuum to afford 450-1 (650 mg, crude) as a yellow oil.

### 2. Synthesis of 450-2

To a solution of 450-1 (550 mg, 2.53 mmol, 2 equiv) and 1-2 (377 mg, 1.27 mmol, 1 equiv) in THF (4 mL) and H₂O (1 mL) was added Cs₂CO₃ (1.24 g, 3.80 mmol, 3 equiv) and dicyclohexyl-[2-(2,4,6-triisopropylphenyl)phenyl]phosphane;methanesulfonate;[2-[2-(methylamino)phenyl]phenyl]palladium(1+) (109 mg, 126 umol, 0.1 equiv), the mixture was stirred at 80° C. for 12 h under nitrogen atmosphere. The combined mixture was diluted with water (10 mL), extracted with EtOAc (10 mL×3). The combined organic layers dried over anhydrous Na2SO₄, filtered and concentrated in vacuum. The residue was purified by silica gel column chromatography eluted with EtOAc/PE (3:1) to afford 450-2 (360 mg, 86.5%) as a yellow oil.

¹H-NMR-450-2: (400 MHz, METHANOL-d₄) δ 8.80 (s, 1H), 8.19 (s, 1H), 6.18 (d, J=0.8 Hz, 1H), 4.31-4.22 (m, 2H), 4.10-4.02 (m, 2H), 3.80 (s, 2H), 2.80 (t, J=6.8 Hz, 2H), 2.54 (s, 2H), 1.87 (t, J=6.9 Hz, 2H), 0.57 (d, J=7.6 Hz, 4H)

### 3. Synthesis of 450-3

To a solution of 450-2 (0.36 g, 1.1 mmol, 1 equiv) in dioxane (3.6 mL) was added HCl (4 M, 3.6 m L, 13.13 equiv), the mixture was stirred at 100° C. for 12 hr. The reaction mixture was adjusted pH to 8 by Sat.NaHCO₃, extracted with DCM (8 mL×3). The combined organic layers were dried over anhydrous Na₂SO₄, filtered and concentrated in vacuum. The residue was purified by silica gel column chromatography eluted with EtOAc/PE. (1:3) to afford 450-3 (230 mg, 74%) as a light yellow oil.

¹H-NMR-450-3: (400 MHz, CHLOROFORM-d) δ 10.01 (d, J=1.2 Hz, 1H), 8.69 (s, 1H), 7.95 (s, 1H), 3.57 (s, 2H), 2.55 (t, J=6.8 Hz, 2H), 2.29 (s, 2H), 1.68-1.60 (m, 2H), 0.34 (d, J=3.2 Hz, 4H)

### 4. Synthesis of 450-4

To a solution of 450-3 (0.23 g, 809 umol, 1 equiv) and 3-(1-(4-methyl-4H-1,2,4-triazol-3-yl)cyclobutyl)aniline (184 mg, 809 umol, 1 equiv) in MeOH (3 mL) was added AcOH (139 uL, 2.43 mmol, 3 equiv), the mixture was stirred at 20° C. for 1 hr, then NaBH₃CN (101 mg, 1.62 mmol, 2 equiv) was added to the mixture, the result mixture was stirred at 20° C. for 1 hr under nitrogen atmosphere. The reaction mixture was diluted with Sat.NaHCO₃ (20 mL), extracted with EtOAc (15 mL×3). The combined organic layers were dried over anhydrous Na₂SO₄, filtered and concentrated in vacuum. The residue was purified by silica gel column chromatography eluted with CH₂Cl₂/MeOH (10:1) to afford 450-4 (350 mg, 87%) as a yellow solid.

¹H-NMR-450-4: (400 MHz, CHLOROFORM-d) δ 8.60 (s, 1H), 7.96-7.89 (m, 2H), 7.12 (t, J=8.0 Hz, 1H), 6.64-6.48 (m, 3H), 4.46 (s, 2H), 3.67 (s, 2H), 3.11 (s, 3H), 2.95-2.84 (m, 2H), 2.78-2.70 (m, 2H), 2.66-2.57 (m, 2H), 2.47 (s, 2H), 2.06-2.00 (m, 2H), 1.79 (t, J=6.8 Hz, 2H), 0.49 (s, 4H)

### 5. Synthesis of 450

To a solution of 450-4 (0.27 g, 543 umol, 1 equiv) in DCM (3.5 mL) was added pyridine (263 uL, 3.26 mmol, 6 equiv), then the mixture was cooled to 0° C., bis(trichloromethyl) carbonate (80.6 mg, 271 umol, 0.5 equiv) was added to the mixture at 0° C., the mixture was stirred at 20° C. for 0.5 h under nitrogen atmosphere. The reaction mixture was diluted with Sat. NaHCO₃ (15 mL), extracted with DCM (8 mL×3). The combined organic layers were dried over anhydrous Na₂SO₄, filtered and concentrated in vacuum. The residue was purified by silica gel column chromatography eluted with CH₂Cl₂/MeOH (10:1) to give crude product. The crude product (300 mg) was purified by Prep-PLC with the following conditions (Column: Phenomenex Luna C18 200*40 mm*10 um; Mobile Phase A: Water (0.2% formic acid), Mobile Phase B: ACN; Flow rate: 50 mL/min; Gradient: 1% B to 45% B in 8 min; Wave Length: 220 nm; RT1 (min): 8.5) to afford 450 (81 mg, 28%) as a yellow solid.

MS-450: (ES, m/z): [M+H]⁺ 523.3. ¹H-NMR-450: (400 MHz, METHANOL-d₄) δ 8.38 (s, 1H), 8.37 (s, 1H), 7.90 (s, 1H), 7.77-7.72 (m, 1H), 7.65-7.53 (m, 2H), 7.37 (d, J=7.2 Hz, 1H), 7.22 (s, 1H), 7.16 (s, 1H), 3.97 (s, 2H), 3.35 (s, 3H), 3.31-3.26 (m, 2H), 3.08-2.99 (m, 4H), 2.89-2.78 (m, 2H), 2.23-2.10 (m, 2H), 2.04 (t, J=7.2 Hz, 2H), 0.73 (d, J=8.4 Hz, 4H).

### Example 440. Synthesis of Compound 451

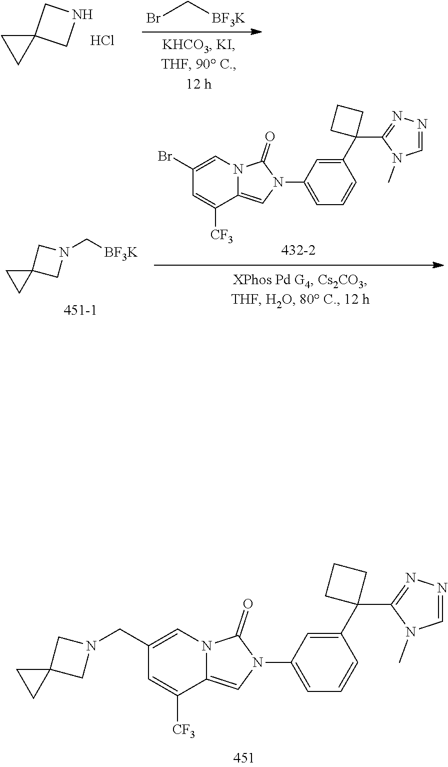

### 1. Synthesis of 451-1

To a solution of potassium;bromomethyl(trifluoro)boranuide (839 mg, 4.18 mmol, 1 equiv), 5-azaspiro[2,3]hexane;hydrochloride (500 rug, 4.18 mmol 1 equiv), KHCO₃ (837 mg 836 mmol, 2 equiv) and KI (69.4 mg, 418, umol, 0.1 equiv) in THF (13 mL) was stirred at 90° C. for 12 h under N₂. The mixture was concentrated under reduced pressure to give a residue. The residue was triturated with acetone at 40° C. for 2 h, then the mixture was filtered and filtrate was concentrated under reduced pressure to afford 451-1 (700 mg, crude) as a white solid.

### 2. Synthesis of 451

To a solution of 432-2 (300 mg, 609 umol, 1 equiv) and 451-1 (309 mg, 1.52 mmol, 2.5 equiv) in THF (8 mL) and H₂O (2 mL) was added Cs₂CO₃ (595.66 mg, 1.83 mmol, 3 equiv) and dicyclohexyl-[2-(2,4,6-triisopropylphenyl)phenyl]phosphane;methanesulfonate;[2-[2-(methylamino)phenyl]phenyl]palladium(1+) (52.4 mg, 60.9 umol, 0.1 equiv). The mixture was stirred at 80° C. for 12 h under nitrogen atmosphere. The reaction mixture was extracted with water (120 mL) and EtOAc (40 mL×3). The combined organic layers were dried over anhydrous Na2SO₄, filtered and the filtrate was concentrated under reduced pressure to give a residue. The residue was purified by silica gel column chromatography eluted with CH₂Cl₂/MeOH (10:1) to give a crude product. The crude product (170 mg) was purified by prep-HPLC with the following conditions (column: Phenomenex Luna C18 200*40 mm*10 um; Mobile Phase A: Water (0.2% formic acid), Mobile Phase B: ACN; Flow rate: 50 mL/min; Gradient: 10% B to 45% B in 8 min; Wave Length: 220 nm; RT1 (min): 8.3) to afford 451 (51 mg, 16.3%) as a yellow solid.

MS-451: (ES, m/z): [M+H]⁺ 509.2. H-NMR-451: (400 MHz, METHANOL-d4) 5=8.51 (s, 1H), 8.36 (s, 1H), 7.87 (s, 1H), 7.75-7.73 (m, 1H), 7.60-7.52 (m, 2H), 7.32 (br d, J=7.6 Hz, 1H), 7.18 (s, 1H), 7.08 (s, 1H), 3.97 (s, 2H), 3.87 (s, 4H), 3.33 (s, 3H), 3.04-2.97 (m, 2H), 2.84-2.77 (m, 2H), 2.18-2.07 (m, 2H), 0.71 (s, 4H).

### Example 441 Synthesis of Compound 452

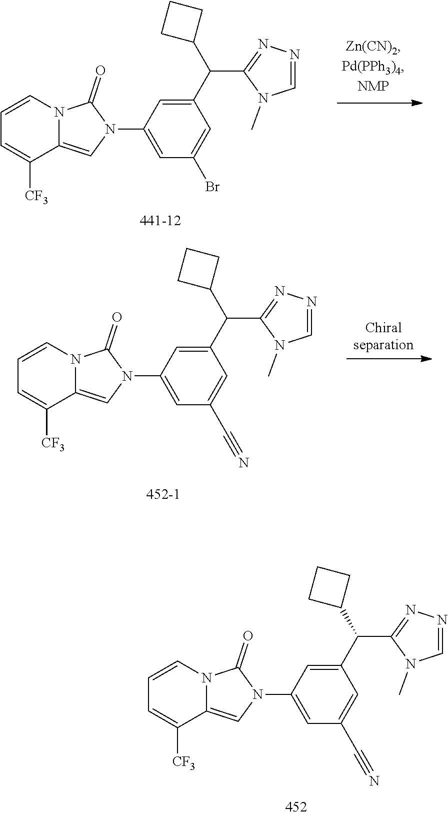 [SMILES: Cn1cnnc1C(c1cc(Br)cc(-n2cc3c(C(F)(F)F)cccn3c2=O)c1)C1CCC1 ; Cn1cnnc1C(c1cc(C#N)cc(-n2cc3c(C(F)(F)F)cccn3c2=O)c1)C1CCC1 ; Cn1cnnc1[C@H](c1cc(C#N)cc(-n2cc3c(C(F)(F)F)cccn3c2=O)c1)C1CCC1]

### 1. Synthesis of 452-1

To a stirred solution of 441-12 (400 mg 0,790 mmol, 1 equiv) and Zn(CN)₂ (371.05 mg, 3.160 mmol, 4 equiv) in NMP (5 mL) was added Pd(PPh₃)4 (91.29 mg, 0.079 mmol, 0.1 equiv) under argon atmosphere. The resulting mixture was stirred for 4 h at 140° C. under argon atmosphere. The mixture was allowed to cool down to room temperature. The reaction was quenched by the addition of water (15 mL) at room temperature. The resulting mixture was extracted with EtOAc (1×15 mL). The combined organic layers were washed with sat. LiCl (aq.) (15 mL) at room temperature, dried over anhydrous Na2SO4. After filtration, the filtrate was concentrated under reduced pressure. The residue was purified by Prep-TLC (CH₂Cl₂/MeOH 15:1) to afford 452-1 (290 mg, 81.13%) as a yellow solid.

### 2. Synthesis of 452

The 452-1 (290 ng) was purified by Chiral separation with the following conditions (Column: CHIRAL ART Cellulose-SB, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 25% B to 25% B in 17 min; Wave Length: 220/254 nm; RT1 (min): 12.43; RT2 (min): 14.79; The first peak was the product; Sample Solvent: EtOH:DCM=1:1-HPLC; Injection Volume: 0.25 mL; Number of Runs: 18) to afford 452 (29.8 mg, 9.77%) as a yellow solid.

LC-MS-452: (ES, m/z): [M+H]⁺ 454. H-NMR-452: 1H NMR (400 MHz, DMSO-d6 ppm) δ 1.69-1.85 (m, 5H), δ 2.06-2.07 (d, 1H), δ 3.21-3.23 (m, 1H), δ 3.48 (s, 3H), δ 4.38-4.40 (d, 1H), δ 6.28-6.31 (t, 1H), δ 7.11-7.13 (d, 1H), δ 7.51 (s, 1H), δ 7.69 (s, 1H), δ 7.78-7.80 (d, 1H), δ 8.25-8.26 (d, 2H), δ 8.36 (s, 1H).

### Example 442 Synthesis of Compound 453

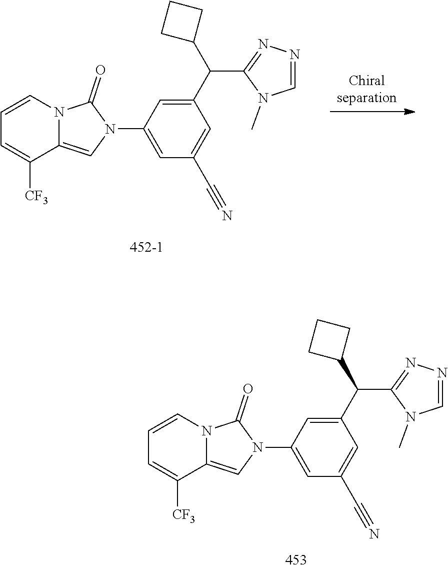 [SMILES: Cn1cnnc1C(c1cc(C#N)cc(-n2cc3c(C(F)(F)F)cccn3c2=O)c1)C1CCC1 ; Cn1cnnc1[C@@H](c1cc(C#N)cc(-n2cc3c(C(F)(F)F)cccn3c2=O)c1)C1CCC1]

### 1. Synthesis of 453

The 452-1 (290 mg) was purified by Chiral separation with the following conditions (Column: CHIRAL ART Cellulose-SB, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 25% B to 25% B in 17 min; Wave Length: 220/254 nm; RT1 (min): 12.43; RT2 (min): 14.79; The second peak is product; Sample Solvent: EtOH:DCM=1:1-HPLC; Injection Volume: 0.25 mL; Number of Runs: 18) to afford 453 (11.6 mg, 3.92%) as a yellow solid.

LC-MS-453: (ES, m/z): [M+H]⁺ 454. H-NMR-453: 1H NMR (400 MHz, DMSO-d6 ppm) δ 1.71-1.80 (m, 5H), δ 2.06-2.07 (d, 1H), δ 3.22-3.23 (m, 1H), δ 3.48 (s, 3H), δ 4.38-4.40 (d, 1H), δ 6.28-6.31 (t, 1H), δ 7.11-7.13 (d, 1H), δ 7.51 (s, 1H), δ 7.69 (s, 1H), δ 7.78-7.80 (d, 1H), δ 8.24-8.25 (d, 2H), δ 8.36 (s, 1H).

### Example 443. Synthesis of Compound 454

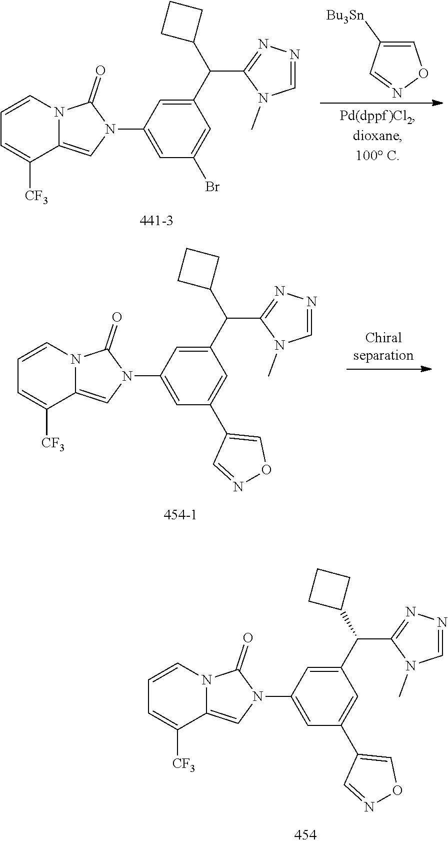

### 1. Synthesis of 454-1

To a stirred solution of 441-3 (850 ng, 1.68 mmol, 1.0 equiv) and 4-(tributylstannyl)-1,2-oxazole (902 mg 252 mmol, 1.5 equiv) in dioxane (15 mL) was added Pd(dppf)Cl2 (137 mg, 0.17 mmol, 0.1 equiv) under nitrogen atmosphere. The resulting mixture was stirred for overnight at 80° C. under nitrogen atmosphere. The resulting mixture was concentrated under reduced pressure. The residue was purified by Prep-TLC (CH2Cl2/MeOH=20:1) to afford 454-1 (230 mg 28%) as a yellow solid.

### 2. Synthesis of 454

The 454-1 (230 mg, 0.47 mmol, 1.0 equiv) was purified by Prep-Chiral-HPLC with the following conditions (Column: CHIRAL ART Cellulose-SB, 2*25 cm, 5 μm; Mobile Phase A: Hex (0.5% 2M NH3-MeOH), Mobile Phase B: EtOH:DCM=1:1; Flow rate: 20 mL/min; Gradient: 25% B to 25% B in 16.5 min; Wave Length: 220/254 n; RT1 (min): 11.39; RT2 (min): 14.18; the first peak was product) to afford 454 (64 mg, 27%) as a yellow solid.

LC-MS-454: (ES, m/z): [M+H]+ 495. H-NMR-454: (400 MHz, DMSO-d6, δ ppm): 1.70-1.82 (m, 5H), 2.08-2.12 (m, 1H), 3.28-3.30 (m, 1H), 3.45 (s, 3H), 4.23-4.28 (d, 1H), 6.28-6.32 (t, 1H), 7.09-7.13 (d, 1H), 7.43 (s, 1H), 7.57 (s, 1H), 7.76-7.80 (m, 1H), 7.83 (s, 1H), 7.89 (s, 1H), 8.34 (s, 1H), 9.22 (s, 1H), 9.52 (s, 1H).

### Example 444. Synthesis of Compound 455

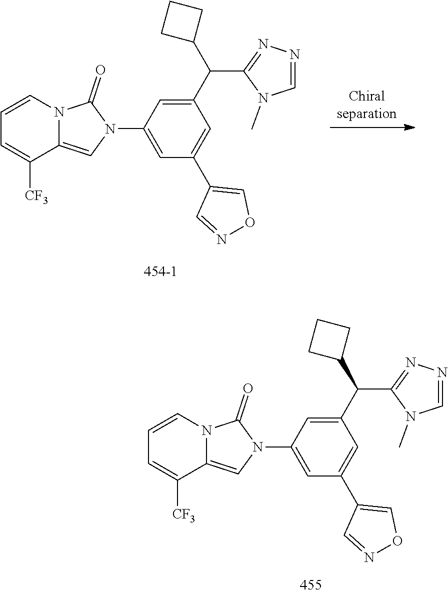 [SMILES: Cn1cnnc1C(c1cc(-c2cnoc2)cc(-n2cc3c(C(F)(F)F)cccn3c2=O)c1)C1CCC1 ; Cn1cnnc1[C@@H](c1cc(-c2cnoc2)cc(-n2cc3c(C(F)(F)F)cccn3c2=O)c1)C1CCC1]

### 1. Synthesis of 455

The 454-1 (230 mg, 0.47 mmol, 1.0 equiv) was purified by Prep-Chiral-HPLC with the following conditions (Column: CHIRAL ART Cellulose-SB, 2*25 cm, 5 μm; Mobile Phase A: Hex (0.5% 2M NH₃-MeOH), Mobile Phase B: EtOH:DCM=1:1; Flow rate: 20 mL/min; Gradient: 25% B to 25% B in 16.5 min; Wave Length: 220/254 nm; RT1 (min): 11.39; RT2 (min): 14.18; the second peak was product) to afford 455 (36 mg, 15%) as a yellow solid.

LC-MS-455: (ES, m/z): [M+H]⁺ 495. H-NMR-455: (400 MHz, DMSO-d6, δ ppm): 1.70-1.74 (m, 5H), 1.75-1.82 (m, 4H), 2.08-2.12 (m, 1H), 3.28-3.30 (m, 1H), 3.47 (s, 3H), 4.25-4.28 (d, 1H), 6.29-6.32 (t, 1H), 7.11-7.13 (d, 1H), 7.43 (s, 1H), 7.58 (s, 1H), 7.78-7.80 (d, 1H), 7.83 (s, 1H), 7.89 (s, 1H), 8.34 (s, 1H), 9.21 (s, 1H), 9.52 (s, 1H).

### Example 445. Synthesis of Compound 456

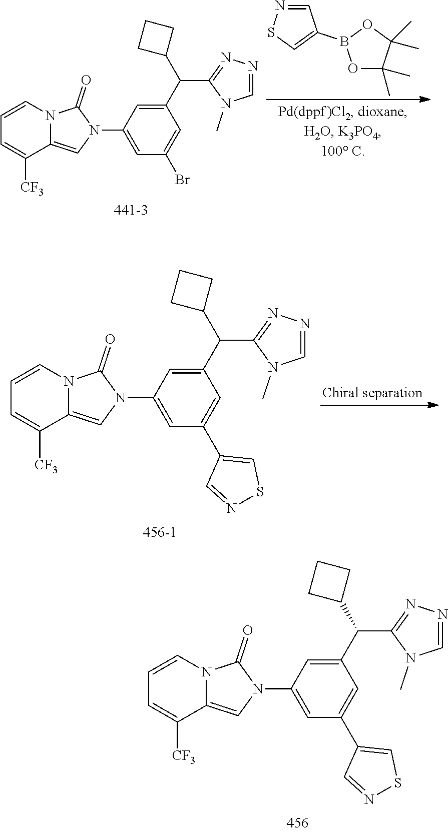

### 1. Synthesis of 456-1

To a stirred mixture of 441-3 (400 mg, 0.790 mmol, 1 equiv) and 4-(4,4,5,5-tetramethyl-1,3,2-dioxaborolan-2-yl)-1,2-thiazole (250.14 mg, 1.185 mmol, 1.5 equiv) in dioxane (4 mL) and H₂O (1 mL) were added K₃PO₄ (335.38 mg, 1.580 mmol, 2 equiv) and Pd(dppf)Cl2 (57.81 mg, 0,079 mmol, 0.1 equiv) under nitrogen atmosphere. The resulting mixture was stirred for overnight at 100° C. under nitrogen atmosphere. The resulting mixture was diluted with water (10 ml). The resulting mixture was extracted with EtOAc (2×10 mL). The combined organic layers were concentrated under reduced pressure. The residue was purified by Prep-TLC (CH2Cl₂/MeOH 20:1) to afford 456-1 (320 mg, 79.34%) as a yellow solid.

### 2. Synthesis of 456

456-1 (320 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 17 min; Wave Length: 220/254 nm; RT1 (min): 9.53: RT2 (min): 12.25; the first peak was product) to afford 456 (114.6 mg, 35.81%) as a yellow solid.

LCMS-456: (ES, m/z): [M+H]⁺ 511. NMR-456: (400 MHz, DMSO, ppm): δ 1.71-1.84 (m, 5H), 2.08-2.13 (m, 1H), 3.26-3.28 (m, 1H), 3.53 (s, 3H), 4.30-4.32 (d, 1H), 6.28-6.32 (t, 1H), 7.10-7.12 (d, 1H), 7.49 (s, 1H), 7.66 (s, 1H), 7.78-7.80 (d, 1H), 7.84 (s, 1H), 7.99 (s, 1H), 8.34 (s, 1H), 9.09 (s, 1H), 9.43 (s, 1H).

### Example 446. Synthesis of Compound 457

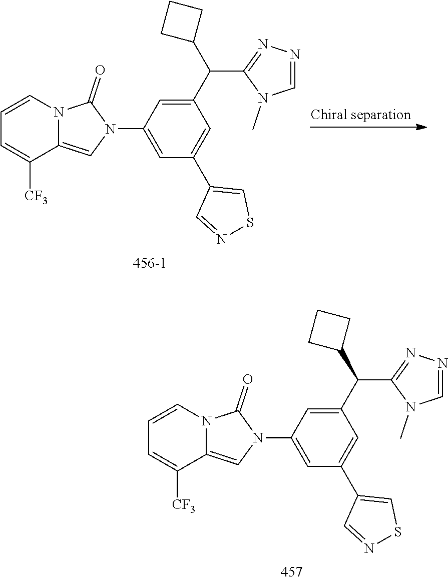 [SMILES: Cn1cnnc1C(c1cc(-c2cnsc2)cc(-n2cc3c(C(F)(F)F)cccn3c2=O)c1)C1CCC1 ; Cn1cnnc1[C@@H](c1cc(-c2cnsc2)cc(-n2cc3c(C(F)(F)F)cccn3c2=O)c1)C1CCC1]

### 1. Synthesis of 457

456-1 (320 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 17 min; Wave Length: 220/254 nm; RT1 (min): 9.53; RT2 (min): 12.25; the second peak was product) to afford 457 (103.0 mg, 32.19%) as a yellow solid.

LCMS-457 (ES, m/z): [M+H]⁺ 511. NMR-457 (400 MHz, DMSO, ppm): δ 1.71-1.84 (m, 5H), 2.08-2.13 (m, 1H), 3.34-3.43 (m, 1H), 3.49 (s, 3H), 4.30-4.33 (d, 1H), 6.28-6.32 (t, 1H), 7.10-7.12 (d, 1H), 7.49 (s, 1H), 7.66 (s, 1H), 7.78-7.80 (d, 1H), 7.84 (s, 1H), 7.99 (s, 1H), 8.34 (s, 1H), 9.09 (s, 1H), 9.43 (s, 1H).

### Example 447. Synthesis of Compound 458

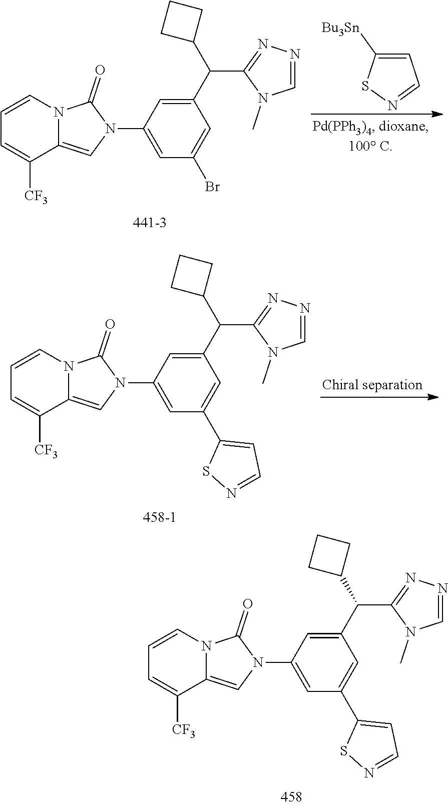

### 1. Synthesis of 458-1

To a stirred solution of 441-3 (400 mg, 0.790 mmol, 1 equiv) and 5-(tributylstannyl)-1,2-thiazole (443.39 mg 1,185 mmol, 1.5 equiv) in dioxane (4 ML) was added Pd(PPh₃)4 (91.29 mg, 0.079 mmol, 0.1 equiv) under nitrogen atmosphere. The resulting mixture was stirred for overnight at 100° C. under nitrogen atmosphere. The resulting mixture was concentrated under reduced pressure. The residue was purified by Prep-TLC (CH₂Cl₂/MeOH 20:1) to afford 458-1 (360 mg, 89.26%) as a yellow solid.

### 2. Synthesis of 458

458-1 (360 mg) was purified by Prep-Chiral-HPLC with the following conditions (Column: CHIRALPAK IG, 2*25 cm, 5 μm; Mobile Phase A: Hex (0.5% 2M NH₃-MeOH, Mobile Phase B: EtOH:DCM=1:1; Flow rate: 20 mL/min; Gradient: 40% B to 40% B in 17 min; Wave Length: 220/254 nm; RT1 (min): 9.53; RT2 (min): 12.25; The first peak was product) to afford 458 (132.6 mg, 36.83%) as a yellow solid.

LCMS-458 (ES, m/z): [M+H]⁺511. NMR-458 (400 MHz, DMSO, ppm): δ 1.73-1.84 (m, 5H), 2.08-2.12 (m, 1H), 3.27-3.29 (m, 1H), 3.50 (s, 3H), 4.37-4.40 (d, 1H), 6.29-6.32 (t, 1H), 7.11-7.13 (d, 1H), 7.51 (s, 1H), 7.60 (s, 1H), 7.79-7.81 (d, 1H), 7.87 (s, 1H), 7.92 (s, 1H), 8.06 (s, 1H), 8.37 (s, 1H), 8.64 (s, 1H).

### Example 448. Synthesis of Compound 459

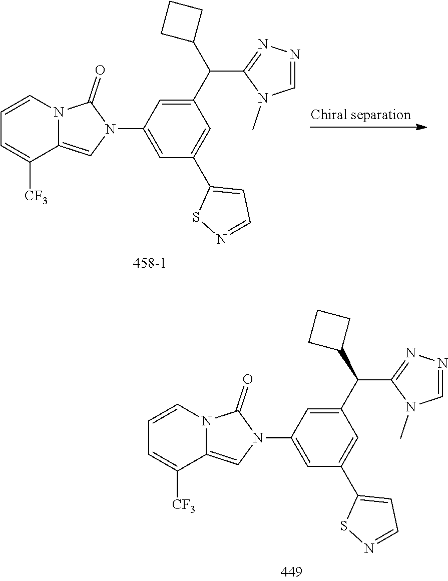 [SMILES: Cn1cnnc1C(c1cc(-c2ccns2)cc(-n2cc3c(C(F)(F)F)cccn3c2=O)c1)C1CCC1 ; Cn1cnnc1[C@@H](c1cc(-c2ccns2)cc(-n2cc3c(C(F)(F)F)cccn3c2=O)c1)C1CCC1]

### Synthesis of 459

458-1 (360 mg) was purified by Prep-Chiral-HPLC with the following conditions (Column: CHIRALPAK IG, 2*25 cm, 5 μm; Mobile Phase A: Hex (015% 2M NH₃-MeOH), Mobile Phase B: EtOH:DCM=1:1; Flow rate: 20 mL/min; Gradient: 40% B to 40% B in 17 min; Wave Length: 220/254 nm; RT1 (min): 9.53; RT2 (min): 12.25; the second peak was product) to afford 459 (69.3 mg, 19.25%) as a yellow solid.

LCMS-459 (ES, m/z): [M+H]⁺ 511. NMR-459 (400 MHz, DMSO, ppm): δ 1.71-1.83 (m, 5H), 2.08-2.11 (m, 1H), 3.27-3.30 (m, 1H), 3.50 (s, 3H), 4.37-4.40 (d, 1H), 6.29-6.32 (t, 1H), 7.11-7.13 (d, 1H), 7.51 (s, 1H), 7.60 (s, 1H), 7.79-7.80 (d, 1H), 7.87 (s, 1H), 7.92 (s, 1H), 8.06 (s, 1H), 8.35 (s, 1H), 8.64 (s, 1H).

### Example 449. Synthesis of Compound 460

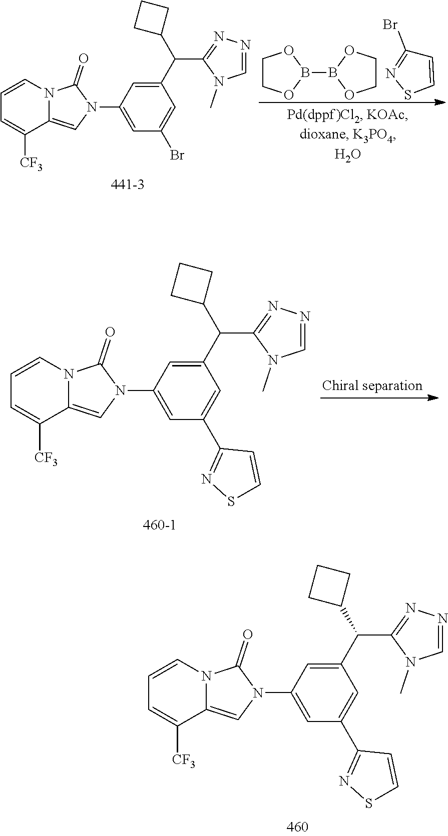

### 1. Synthesis of 460-1

To a stirred mixture of 441-3 (1 g, 1.97 mmol, 1.0 equiv) and bis(pinacolato)diboron (0.5 g, 1.97 mmol, 1.0 equiv) in dioxane (10 mL) was added KOAc (190 mg, 1.97 mmol, 1.0 equiv) and Pd(dppf)Cl₂ (140 mg, 0.20 mmol, 0.1 equiv) under nitrogen atmosphere. The resulting mixture was stirred for 4 h at 80° C. under nitrogen atmosphere. To the above mixture was added H₂O (3 mL), 3-bromo-1,2-thiazole (490 mg, 2.96 mmol, 1.5 equiv), K₃PO₄ (840 mg, 3.95 mmol, 2.0 equiv) and Pd(dppf)Cl₂ (140 mg, 0.20 mmol, 0.1 equiv) was added under nitrogen atmosphere. The resulting mixture was stirred overnight at 80° C. The resulting mixture was diluted with water (30 mL). The resulting mixture was extracted with EtOAc (3×20 mL). The combined organic layers were concentrated under reduced pressure. The resulting mixture was concentrated under reduced pressure. The residue was purified by Prep-TLC (CH₂Cl₂/MeOH=25:1) to afford 460-1 as a yellow solid.

### 2. Synthesis of 460

The 460-1 (350 mg, 0.69 mmol, 1.0 equiv) was purified by Prep-HPLC with the following conditions (Column: CHIRAL ART Cellulose-SB, 2*25 cm, 5 μm; Mobile Phase A: Hex (0.5% 2M NH₃-MeOH), Mobile Phase B: EtOH:DCM=1:1; Flow rate: 20 mL/min; Gradient: 25% B to 25% B in 15 min; Wave Length: 220/254 nm; RT1 (min): 8.90; RT2 (min): 11.87; the first peak was product) to afford 460 (102.5 mg, 28%) as a yellow solid.

LC-MS-460 (ES, m/z): [M+H]⁺ 511. H-NMR-460 (400 MHz, DMSO-d6, δ ppm): 1.76-1.88 (m, 5H), 2.11-2.14 (m, 1H), 3.25-3.27 (m, 1H), 3.47 (s, 3H), 4.36-4.39 (d, 1H), 6.29-6.32 (t, 1H), 7.11-7.13 (d, 1H), 7.49 (s, 1H), 7.79-7.81 (d, 1H), 7.90-7.91 (d, 1H), 8.01 (s, 1H), 8.26 (s, 1H), 8.35 (s, 1H), 9.20-9.21 (d, 1H).

### Example 450. Synthesis of Compound 461

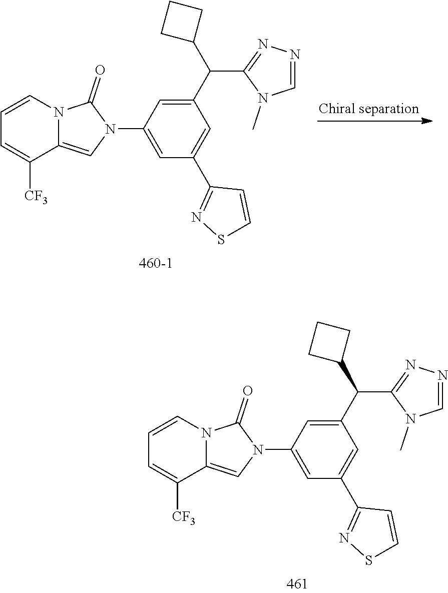 [SMILES: Cn1cnnc1C(c1cc(-c2ccsn2)cc(-n2cc3c(C(F)(F)F)cccn3c2=O)c1)C1CCC1 ; Cn1cnnc1[C@@H](c1cc(-c2ccsn2)cc(-n2cc3c(C(F)(F)F)cccn3c2=O)c1)C1CCC1]

### 1. Synthesis of 461

The 460-1 (350 mg) was purified by Prep-Chiral-HPLC with the following conditions (Column: CHIRAL ART Cellulose-SB, 2*25 cm, 5 um; Mobile Phase A: Hex (0.5% 2M NH₃-MeOH), Mobile Phase B: EtOH:DCM=1:1; Flow rate: 20 mL/min; Gradient: 25% B to 25% B in 15 min; Wave Length: 220/254 nm; RT1 (main): 8.90; RT2 (min): 11.87; the second peak was product) to afford crude product. The crude product (74 mg) was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 35% B to 57% B in 8 min, 57% B; Wave Length: 220 nm; RT1 (min): 7.28) to 461 (28 mg, 8%) as a yellow solid.

LC-MS-461 (ES, m/z): [M+H]⁺ 511. H-NMR-461 (400 MHz, DMSO-d6, δ ppm): 1.76-1.88 (m, 5H), 2.11-2.14 (m, 1H), 3.25-3.27 (m, 1H), 3.47 (s, 3H), 4.36-4.39 (d, 1H), 6.29-6.32 (t, 1H), 7.11-7.13 (d, 1H), 7.49 (s, 1H), 7.79-7.81 (d, 1H), 7.90-7.91 (d, 1H), 8.01 (s, 1H), 8.26 (s, 1H), 8.35 (s, 1H), 9.20-9.21 (d, 1H).

### Example 451. Synthesis of Compound 462

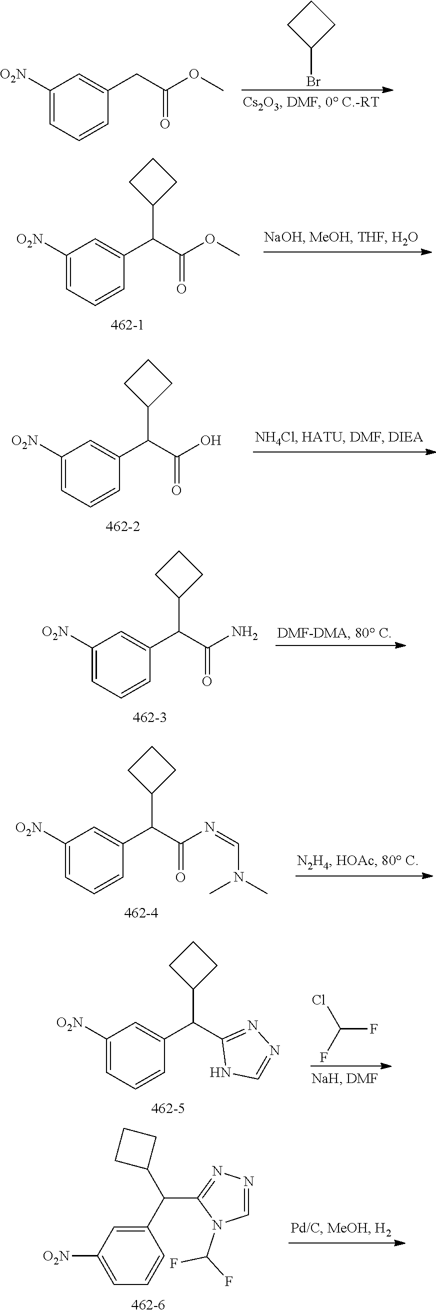

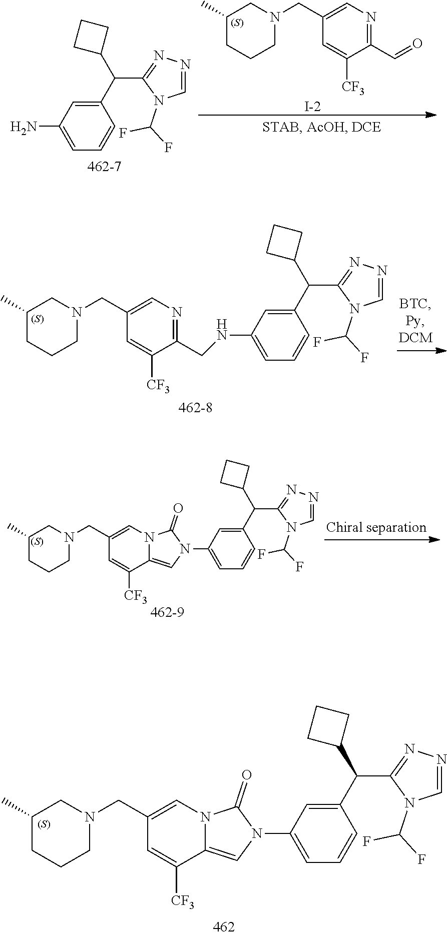

### 1. Synthesis of 462-1

A solution of methyl 2-(3-nitrophenyl) acetate (70 g, 358.654 mmol, 1 equiv) in DMF (700 mL) was treated with Cs₂CO₃ (584.28 g, 1793.270 mmol, 5 equiv) for 1 h at 0° C. under nitrogen atmosphere. To the above mixture was added bromocyclobutane (145.26 g, 1075.962 mmol, 3 equiv) at room temperature. The resulting mixture was stirred for overnight at room temperature under nitrogen atmosphere. The resulting mixture was filtered the filter cake was washed with MTBE (2×500 mL). The filtrate was concentrated under reduced pressure. The crude product was re-crystallized from Heptane/MTBE (5:1 600 mL) to afford 462-1 (61 g, 68:23%) as a Brown yellow solid.

### 2. Synthesis of 462-2

To a stirred mixture of H12 (120 mL) and THF (120 mL) in MeOH (360 mL) were added 462-1 (61 g, 244.718 mmol, 1 equiv) and NaOH (19.58 g, 489.436 mmol, 2 equiv) at room temperature. The resulting mixture was stirred for 1 h at room temperature. The mixture was acidified to pH 7 with conc.HCl (100 mL). The aqueous layer was extracted with EtOAc (3×500 mL). The resulting liquid was dried over anhydrous Na₂SO₄. The resulting mixture was concentrated under reduced pressure. This resulted in 462-2 (56 g, 94.36%) as a light red solid.

### 3. Synthesis of 462-3

To a stirred mixture of 462-2 (56 g, 238.056 mmol, 1 equiv) and DIEA (61.54 g, 476.112 mmol, 2 equiv) in DMF (600 ml) were added HATU (181.03 g, 476.112 mmol, 2 equiv) and NH4Cl (38.20 g, 714,168 mol, 3 equiv) at room temperature under nitrogen atmosphere. The resulting mixture was stirred for overnight at room temperature under nitrogen atmosphere. The resulting mixture was diluted with water (2 L). The aqueous layer was extracted with EtOAc (3×500 ml). The resulting mixture was concentrated under reduced pressure. The residue was purified by silica gel column chromatography, eluted with CH₂Cl₂/MeOH (10:1) to afford 462-3 (55 g, 91.72%) as a Brown yellow solid,

### 4. Synthesis of 462-4

To a stirred solution of 462-3 (55 g, 234.787 mmol, 1 equiv) in DMF-DMA (550 mL) at room temperature. The resulting mixture was stirred for overnight at 80° C. under nitrogen atmosphere. The resulting mixture was diluted with water (500 ml). The aqueous layer was extracted with EtOAc (3×200 mL). The resulting mixture was concentrated under reduced pressure. The residue was purified by silica gel column chromatography, eluted with CH₂Cl₂/MeOH (10:1) to afford 462-4 (50 g, 67.71%) as a Brown yellow solid.

### 5. Synthesis of 462-5

To a stirred solution of 462-4 (50 g, 172.810 mmol, 1 equiv) in HOAc (500 mL) at room temperature. To the above mixture was added hydrazine hydrate (400 mL, 8065.360 mmol, 46.67 equiv, 98%) at room temperature. The resulting mixture was stirred for overnight at 80° C. under nitrogen atmosphere. The resulting mixture was diluted with water (500 mL). The aqueous layer was extracted with EtOAc (3×300 mL). The resulting mixture was concentrated under reduced pressure. The residue was purified by silica gel column chromatography, eluted with CH₂Cl₂/MeOH (10:1) to afford 462-5 (40 g, 86.93%) as a Brown yellow solid.

### 6. Synthesis of 462-6

A solution of 462-5 (10 g, 38.718 mmol, 1 equiv) in DMF (100 mL) was treated with NaH (4.65 g, 193.590 mmol, 5 equiv) for 1 h at 0° C. To the above mixture was added chlorodifluoromethane (6270 g, 77.436 mmol, 2 equiv) at room temperature. The resulting mixture was stirred for additional overnight at room temperature. The resulting mixture was diluted with water (500 mL). The aqueous layer was extracted with EtOAc (3×200 mL). The resulting mixture was concentrated under reduced pressure. The residue was purified by silica gel column chromatography, eluted with PE/EA (5:1) to afford 462-6 (5 g, 40.21%) as a yellow green solid.

### 7. Synthesis of 462-7

To a solution of 462-6 (5 g, 16.219 mmol, 1 equiv) in 1.0 mL MeOH was added Pd/C (10%, Ig) under nitrogen atmosphere in a 250 mL round-bottom flask. The mixture was hydrogenated at room temperature for 3 h under hydrogen atmosphere using a hydrogen balloon, filtered through a Celite pad and concentrated under reduced pressure. This resulted in 462-7 (3 g, 64.47%) as a yellow green solid.

### 8. Synthesis of 462-8

A solution of 462-7 (1 g, 3,593 mmol, 1 equiv) in DCE (10 mL) was treated with 1-2 (1.03 g, 3.593 mml, 1 equiv) at room temperature under nitrogen atmosphere followed by the addition of AcOH (0.22 g, 3.593 mmol, 1 equiv) and STAB (2.28 g, 10,779 mmol, 3 equiv) at room temperature. The resulting mixture was stirred for 2 h at room temperature. The resulting mixture was diluted with water (20 mL). The aqueous layer was extracted with CH₃Cl (3×10 mL). The resulting mixture was concentrated under reduced pressure. The residue was purified by Prep-TLC (CH₂Cl₂/MeOH 10:1) to afford 462-8 (1.2 g, 60.88%) as a yellow solid.

### 9. Synthesis of 462-9

A solution of 462-8 (600 mg, 1.094 mmol, 1 equiv) in DCM (6 mL) was treated with pyridine (519.06 mg, 6.564 mmol, 6 equiv) at 0° C. followed by the addition of triphosgene (19432 mg, 0.656 mmol, 0.6 equiv) at room temperature. The resulting mixture was stirred for 10 min at room temperature. The residue was acidified to pH 7 with saturated NaHCO₃ (aq.) (5 mL). The aqueous layer was extracted with CH₂Cl₂ (3×3 mL). The residue was purified by Prep-TLC (CH2Cl2/MeOH 15:1) to afford 462-9 (250 mg, 38.59%) as a yellow solid.

### 10. Synthesis of 462

The 462-9 (250 mg, 0.435 mmol, 1 equiv) was purified by Chiral separation with the following conditions (Column: CHIRALPAK IC, 2*25 nm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 25% B to 25% B in 11 min; Wave Length: 220/254 am; RT1 (min): 8.34; RT2 (min): 9.85; The second peak was product. Sample Solvent: EtOH:DCM=1:1-HPLC; Injection Volume: 0.1 mL; Number of Runs: 18) to afford 462 (100 mg, 39.20%) as a yellow solid.

LC-MS-462 (ES, m/z): [M+H]⁺ 575. H-NMR-462 (400 MHz, DMSO-d6, ppm): 50.82-0.84 (m, 4H), 51.58-1.67 (d, 1H), 51.67-1.82 (m, 5H), 61.82-1.99 (m, 5H), 62.00-2.15 (d, 1H), 62.75-2.87 (s, 2H), 63.18-3.33 (m, 3H), 64.41-4.43 (d, 1H), 67.01 (d, 1H), 67.09-7.23 (d, 1H), 67.31 (s, 1H), 57.39-7.41 (d, 1H), 67.47-7.65 (d, 1H), 67.71 (d, 1H), 67.75-7.82 (d, 1H), 67.85-8.02 (d, 1H), 68.89-8.94 (d, 1H).

### Example 452. Synthesis of Compound 463

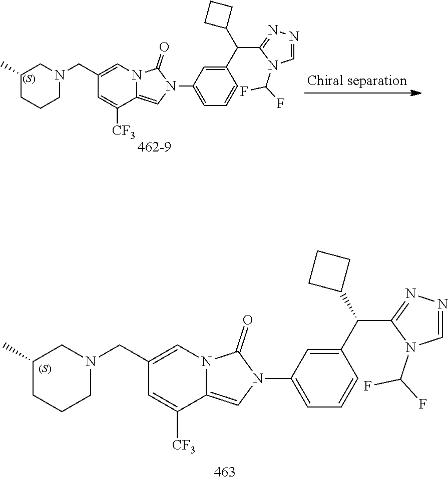

### 1. Synthesis of 463

The 462-9 (250 mg, 0.435 mmol, 1 equiv) was purified by Chiral separation with the following conditions (Column: CHIRALPAK IC, 2*25 cm, 5 man; Mobile Phase A: Hex (0.5% 2 NH₃-MeOH)-HPLC, Mobile Phase B: EtOH:DCM=1:1-HPLC; Flow rate: 20 mL/min; Gradient: 25% B to 25% B in 11 min; Wave Length: 220/254 nm; RT1 (min): 8.34; RT2 (min): 9.85; The first peak was product. Sample Solvent: EtOH:DCM=1:1-HPLC; Injection Volume: 0.1 mL; Number of Runs: 18) to afford 463 (77.4 mg, 30.34%) as a yellow solid.

LC-MS-463 (ES, m/z): [M+H]⁺ 575/H-NMR-463 (400 MHz, DMSO-d6, ppm): 60.76-0.94 (m, 4H), 61.41 (d, 1H), 61.52-1.85 (m, 5H), 61.86-1.94 (m, 5H), 61.96-2.05 (d, 1H), 62.72-2.85 (s, 2H), 63.12-3.30 (m, 3H), 54.32-4.51 (d, 1H), 66.93-7.01 (d, 1H), 67.08-7.15 (d, 1H), 67.29 (d, 1H), 67.35-7.51 (d, 1H), 67.66-7.71 (d, 1H), 67.72-7.81 (d, 1H), 67.82-7.85 (d, 1H), 67.86-8.08 (d, 1H), 68.94 (d, 1H).

### Example 453. Synthesis of Compound 464

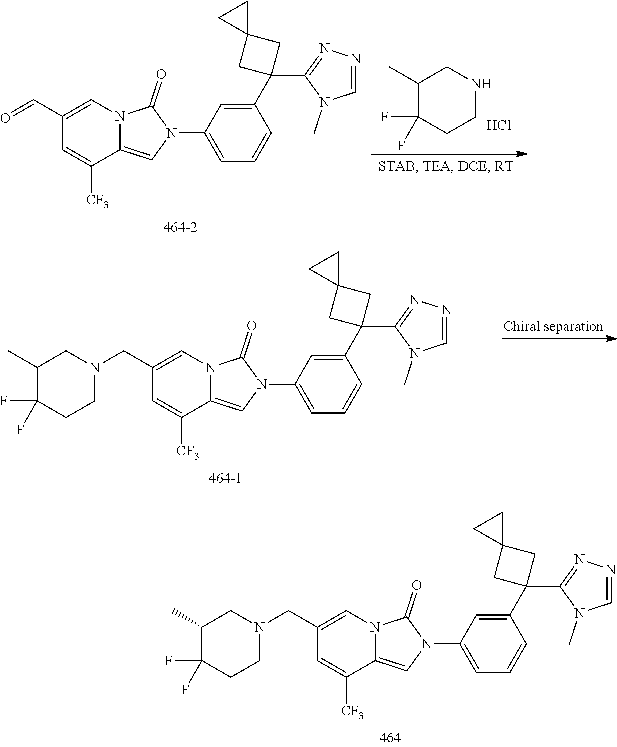

### 1. Synthesis of 464-1

To a stirred solution of 464-2 (700 ng, 1.497 mmol, 1 equiv) and 4,4-difluoro-3-methylpiperidine hydrochloride (385.50 mg, 2.246 mmol 1.5 equiv) in DCE (10 mL) were added TEA (303.07 mg, 2.994 mmol, 2 equiv) at room temperature. The resulting mixture was stirred for 1 h at room temperature. To the above mixture was added STAB (634.75 mg, 2,994 mmol, 2 equiv) at room temperature. The resulting mixture was stirred for additional overnight at room temperature. The reaction was quenched with with saturated NH₄Cl (aq.) (80 mL) at room temperature. The aqueous layer was extracted with DCM (2×50 mL). The resulting mixture was concentrated under vacuum. The residue was purified by Prep-TLC (DCM/MeOH 20:1) to afford 464-1 (430 mg, 47.48%) as a yellow solid.

### 2. Synthesis of 464

The 464-1 (430 mg) was purified by Prep-Chiral-HPLC with the following conditions (Column: CHIRALPAK IC, 2*25 cm, 5 μm; Mobile Phase A: Hex (0.5% 2M NH₃-MeOH) Mobile Phase B: EtOH:DCM=1:1; Flow rate: 20 mL/min; Gradient: 60% B to 60% B in 14 min; Wave Length: 220/254 nm; RT1 (min): 6.86; RT2 (min): 9.75, the first peak is product) to afford 464 (170.6 mg, 38.37%) as a yellow solid.

LC-MS-464 (ES, m/z): [M+H]⁺ 587. H-NMR-464 (400 MHz, DMSO-d6, δ ppm): 0.45-0.49 (m, 2H), 0.55-0.59 (m, 2H), 0.92-0.94 (d, 3H), 1.88-2.18 (m, 4H), 2.26-2.31 (m, 1H), 2.75-2.82 (m, 4H), 3.17-3.22 (m, 2H), 3.24 (s, 3H), 3.35 (s, 2H), 7.02 (s, 1H), 7.18-7.21 (d, 1H), 7.36 (s, 1H), 7.49-7.53 (m, 1H), 7.63-7.65 (d, 1H), 7.72 (s, 1H), 7.83 (s, 1H), 8.39 (s, 1H).

### Example 454. Synthesis of Compound 465

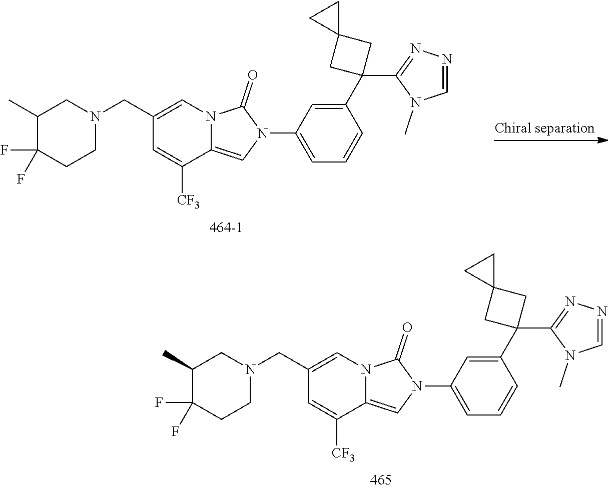 [SMILES: CC1CN(Cc2cc(C(F)(F)F)c3cn(-c4cccc(C5(c6nncn6C)CC6(CC6)C5)c4)c(=O)n3c2)CCC1(F)F ; C[C@H]1CN(Cc2cc(C(F)(F)F)c3cn(-c4cccc(C5(c6nncn6C)CC6(CC6)C5)c4)c(=O)n3c2)CCC1(F)F]

### 1. Synthesis of 465

464-1 (430 mg) was purified by Prep-Chiral-HPLC with the following conditions (Column: CHIRALPAK IC, 2*25 cm, 5 n; Mobile Phase A: Hex (0.5% 2M NH₃-MeOH), Mobile Phase B: EtOH:DCM=1:1; Flow rate: 20 ml/min; Gradient: 60% B to 60% B in 14 min; Wave Length: 220/254 nm; RT1 (min): 6.86; RT2 (min): 9.75, the second peak is product) to afford 465 (143.6 mg, 32.86%) as a yellow solid.

LC-MS-465 (ES, m/z): [M+H]⁺ 587. H-NMR-465 (400 MHz, DMSO-d6, δ ppm): 0.45-0.49 (m, 2H), 0.55-0.59 (m, 2H), 0.92-0.94 (d, 3H), 1.88-2.18 (m, 4H), 2.26-2.31 (m, 1H), 2.75-2.82 (m, 4H), 3.17-3.22 (m, 2H), 3.24 (s, 3H), 3.35 (s, 2H), 7.02 (s, 1H), 7.18-7.21 (d, 1H), 7.36 (s, 1H), 7.49-7.53 (m, 1H), 7.63-7.65 (d, 1H), 7.72 (s, 1H), 7.83 (s, 1H), 8.39 (s, 1H).

### Example 455. Synthesis of Compound 466

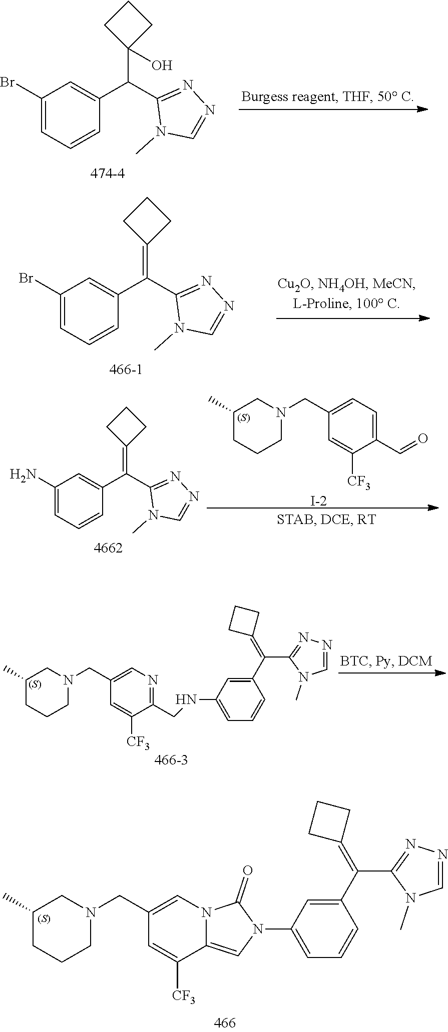

### 1. Synthesis of 466-1

To a stirred solution of 474-4 (500 mg, 1.552 mmol, 1 equiv) in THF (10 mL) was added Burgess reagent (369.79 mg, 1.552 mmol, 1 equiv) at room temperature. The resulting mixture was stirred overnight at 50° C. The resulting mixture was diluted with water (50 mL). The aqueous layer was extracted with EtOAc (2×30 mL). The resulting mixture was concentrated under vacuum. The residue was purified by Prep-TLC (DCM/MeOH 20:1) to afford 466-1 (300 mg, 57.20%) as a light yellow solid.

### 2. Synthesis of 466-2

Into a 20 mL pressure tank reactor were added 466-1 (300 mg, 0986 mmol, 1 equiv), Cu₂O (28.22 mg, 0.197 mmol, 0.2 equiv), NH₄OH (3 mL), MeCN (3 mL) and L-Proline (11.35 mg, 0.099 mmol, 0.1 equiv) at room temperature. The resulting mixture was stirred overnight at 100° C. The mixture was allowed to cool down to room temperature. The resulting mixture was filtered, the filter cake was washed with MeOH (10 mL). The filtrate was concentrated under reduced pressure. The residue was purified by Prep-TLC (DCM/MeOH 12:1) to afford 466-2 (180 mg, 68.35%) as a yellow oil.

### 3. Synthesis of 466-3

To a stirred solution of 466-2 (180 mg, 0.749 mmol, 1 equiv) and 1-2 (235.89 mg, 0.824 mmol, 1.1 equiv) in DCE (5 mL) were added STAB (317.50 mg, 1.498 mmol, 2 equiv) at room temperature. The resulting mixture was stirred for 5 h at room temperature. The reaction was quenched with NaHCO₃ (aq.) (30 mL) at room temperature. The aqueous layer was extracted with DCM (2×20 mL). The resulting mixture was concentrated under vacuum. The residue was purified by Prep-TLC (DCM/MeOH 20:1) to afford 466-3 (240 mg, 59.61%) as an off-white solid.

### 4. Synthesis of 466

To a stirred solution of 466-3 (220 mg, 0.431 mmol, 1 equiv) and Pyridine (340.81 mg, 4.310 mmol, 10 equiv) in DCM (5 mL) was added Triphosgene (51.14 mg, 0.172 mmol, 0.4 equiv) at room temperature. The resulting mixture was stirred for 10 min at room temperature. The reaction was quenched with NaHCO₃ (aq.) (30 mL) at room temperature. The aqueous layer was extracted with DCM (2×20 mL). The residue was purified by Prep-TLC (DCM/MeOH 20:1) to afford 466 (141.3 mg, 59.89%) as a yellow solid,

LC-MS-466 (ES, m/z): [M+H]⁺ 537

H-NMR-466 (400 MHz, DMSO-d6, δ ppm): 0.81-0.95 (m, 4H), 1.41-1.51 (m, 1H), 1.53-1.73 (m, 4H), 1.86-1.91 (m, 1H), 2.04-2.12 (m, 2H), 2.67-2.78 (m, 4H), 3.14-3.17 (m, 2H), 3.24 (s, 2H), 3.32 (s, 3H), 7.00 (s, 1H), 7.02-7.04 (d, 1H), 7.32 (s, 1H), 7.46-7.50 (m, 1H), 7.62-7.70 (m, 2H), 7.71 (s, 1H), 8.51 (s, 1H).

### Example 456. Synthesis of Compound 467

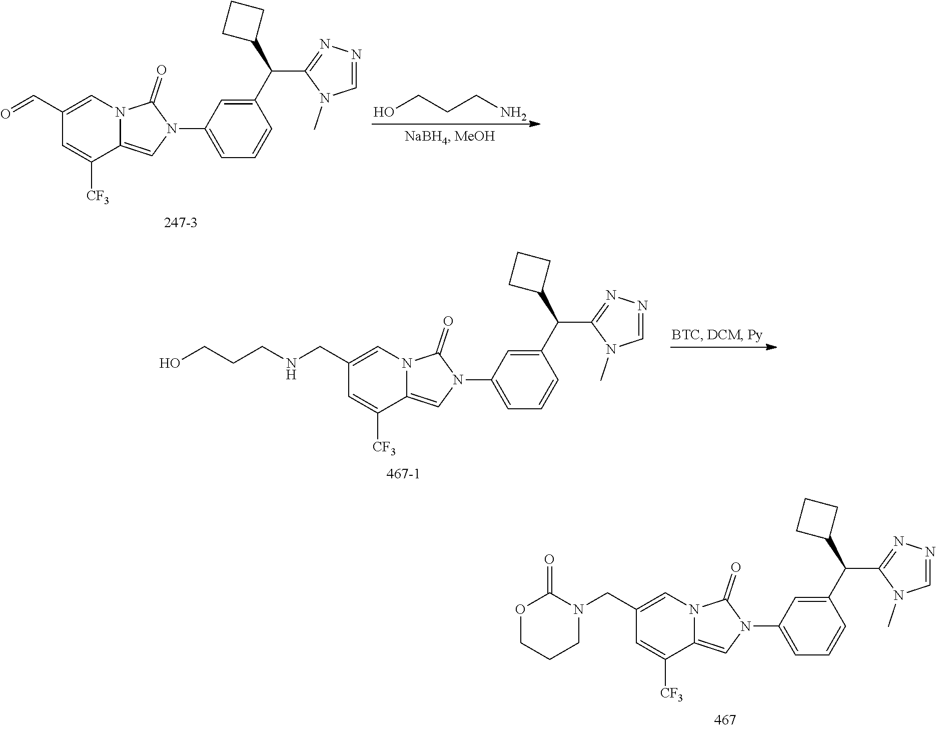

### 1. Synthesis of 467-1

Into a 100 mL round-bottom flask were added 247-3 (1 g, 2.196 mmol, 1 equiv), propanolamine (0.49 g, 6.588 mmol, 3 equiv) and MeOH (20 mL) at room temperature. The mixture was stirred for overnight at room temperature. To the above mixture was added NaBH₄ (0.25 g, 6.588 mmol, 3 equiv) at room temperature. The resulting mixture was stirred for 30 min at room temperature. The reaction was quenched with sat. NH₄Cl (aq.) (100 mL) at room temperature. The aqueous layer was extracted with EtOAc (3×40 mL). The resulting mixture was concentrated under vacuum. The residue was purified by Prep-TLC (DCM/MeOH=10:1) to afford 467-1 (300 mg, 24.43%) as a yellow solid.

### 2. Synthesis of 467

Into a 100 mL round-bottom flask were added 467-1 (650 mg, 1.263 mmol, 1 equiv), DCM (20 mL) and pyridine (799.37 mg, 10.104 mmol, 8 equiv) at room temperature. To the above mixture was added triphosgene (187.42 mg, 0.631 mmol, 0.5 equiv) at room temperature. The resulting mixture was stirred for 3 min at room temperature. The reaction was quenched with sat. NaHCO₃ (aq.) (100 mL) at room temperature. The aqueous layer was extracted with EtOAc (3×60 mL). The resulting mixture was concentrated under vacuum. The residue was purified by Prep-TLC (DCM/MeOH=10:1) to afford crud product. The crude product (200 mg) was purified by Prep-HPLC with the following conditions (Column: X Bridge Prep OBD C18 Column, 30*150 mm, 5 μm; Mobile Phase A: water (10 mmol/L NH₄HCO₃), Mobile Phase B: MeOH-HPLC; Flow rate: 60 mL/min; Gradient: 40% B to 70% B in 8 min; Wave Length: 254 nm; RT1 (min): 7.83) to afford 467 (43.6 mg, 6.33%) as a yellow solid.

LC-MS-467 (ES, m/z): [M+H]⁺ 541. H-NMR-467 1H NMR (400 MHz, MeOD ppm) δ 1.71-1.78 (m, 1H), δ 1.82-1.99 (m, 4H), δ 2.06-2.11 (m, 2H), δ 2.25-2.27 (m, 1H), δ 3.27-3.29 (m, 1H), δ 3.33-3.39 (m, 2H), δ 3.58 (s, 3H), δ 4.28-4.34 (m, 3H), δ 4.35-4.38 (m, 2H), δ 7.07 (s, 1H), δ 7.15 (s, 1H), δ 7.27-7.29 (d, 1H), δ 7.48-7.52 (t, 1H), δ 7.64-7.66 (m, 2H), δ 7.75 (s, 1H), δ 8.37 (s, 1H).

### Example 457. Synthesis of Compound 468

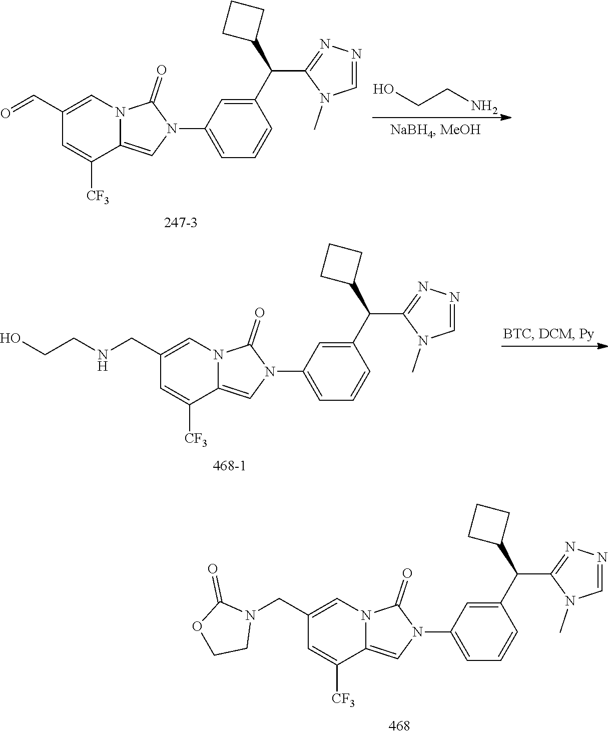

### 1. Synthesis of 468-1

Into a 100 mL round-bottom flask were added 247-3 (600 mg, 1.317 mmol, 1 equiv) propanolamine (0.49 g, 6.588 mmol, 3 equiv), ethanolamine (241.42 mg, 3,951 mmol, 3 equiv) and MeOH (15 mL) at room temperature. The mixture was stirred for overnight at room temperature. To the above mixture was added NaBH₄ (149.51 mg, 3.951 mmol, 3 equiv) at room temperature. The resulting mixture was stirred for 30 min at room temperature. The reaction was quenched with sat. NH₄Cl (aq.) (100 mL) at room temperature. The aqueous layer was extracted with EtOAc (3×40 mL). The resulting mixture was concentrated under vacuum. The residue was purified by Prep-TLC (DCM/MeOH=10:1) to afford 468-1 (200 mg, 27.90%) as a yellow solid.

### 2. Synthesis of 468

Into a 100 mL round-bottom flask were added 468-1 (300 mg, 0.599 mmol, 1 equiv), DCM (10 mL) and pyridine (379.28 mg, 4.792 mmol, 8 equiv) at room temperature. To the above mixture was added triphosgene (88.93 mg, 0.299 mmol, 0.5 equiv) at room temperature. The resulting mixture was stirred for 3 min at room temperature. The reaction was quenched with sat. NaHCO₃ (aq.) (100 mL) at room temperature. The aqueous layer was extracted with EtOAc (3×50 mL). The resulting mixture was concentrated under vacuum. The residue was purified by Prep-TLC (DCM/MeOH=10:1) to afford crud product. The crude product (150 mg) was purified by Prep-HPLC with the following conditions (Column: X Bridge Prep OBD C18 Column, 30*150 mm, 5 μm; Mobile Phase A: water (10 mmol/L NH₄HCO₃), Mobile Phase B: MeOH-HPLC; Flow rate: 60 mL/min; Gradient: 46% B to 65% B in 8 min; Wave Length: 254 ran; RT1 (min): 7.83) to afford 468 (24.7 mg, 7.62%) as a yellow solid.

LC-MS-468 (ES, m/z): [M+H]⁺ 527. H-NMR-468: 1H NMR (400 MHz, MeOD ppm) δ 1.75-1.82 (m, 1H), δ 1.86-1.96 (m, 4H), δ 2.24-2.29 (m, 1H), δ 3.56 (s, 3H), δ 3.60-3.64 (m, 2H), δ 4.28-4.31 (m, 3H), δ 4.38-4.43 (m, 2H), δ 6.99 (s, 1H), δ 7.17 (s, 1H), δ 7.27-7.29 (d, 1H), δ 7.48-7.52 (t, 1H), δ 7.64-7.66 (m, 2H), δ 7.78 (s, 1H), δ 8.37 (s, 1H).

### Example 458. Synthesis of Compound 469

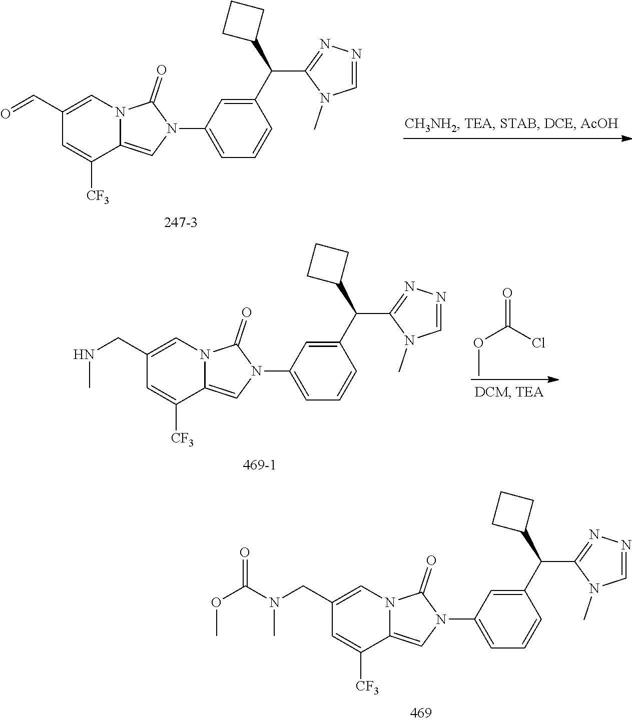

### 1. Synthesis of 469-1

Into a 100 mL 3-necked round-bottom flask were added 247-3 (1 g, 2,196 mmol, 1 equiv), CH₃NH₂ (0.20 g, 6.588 mmol, 3 equiv), STAB (1.40 g, 6.588 mmol, 3 equiv), AcOH (0.13 g, 2.196 mmol, 1 equiv) and DCE (10 mL) at room temperature. The resulting mixture was stirred for overnight at room temperature. The reaction was quenched with sat. NaHCO₃ (aq.) (20 mL) at room temperature. The aqueous layer was extracted with CH₂Cl₂ (3×20 mL). The resulting mixture was concentrated under reduced pressure. The residue was purified by Prep-TLC (CH₂Cl₂/MeOH 10:1) to afford 469-1 (500 mg, 45.50%) as a yellow solid,

### 2. Synthesis of 469

Into a 20 mL sealed tube were added 469-1 (240 mg, 0.510 mmol, 1 equiv), DCM (4 mL), TEA (154.85 mg, 1.530 mmol, 3 equiv) and methyl chloroformate (53.02 mg, 0.561 mmol, 1.1 equiv) at room temperature. The final reaction mixture was irradiated with microwave radiation for 2 h at room temperature. The reaction was quenched with water at room temperature. The aqueous layer was extracted with CH₂Cl₂ (3×5 mL). The resulting mixture was concentrated under reduced pressure. The residue was purified by Prep-TLC (CH₂Cl₂/MeOH 15:1). The crude product (180 mg) was purified by Prep-HPLC with the following conditions (Column: XBridge Shield RP18 OBD Column, 30*150 mm, 5 μm; Mobile Phase A: Water (10 mmol/L NH4HCO3). Mobile Phase B: ACN; Flow rate: 60 mL/min; Gradient: 30% B to 50% B in 8 min; Wave Length: 220 nm; RT1 (min): 7.42) to afford 469 (115.1 mg, 42.35%) as a yellow solid.

LC-MS-469 (ES, m/z): [M+H]⁺ 529. H-NMR-469 (400 MHz, DMSO, δ ppm): 1.50-1.89 (m, 5H), 2.04-2.23 (s, 1H), 2.78-2.90 (s, 3H), 3.14-3.30 (s, 1H), 3.41-3.48 (s, 3H), 3.59-3.69 (s, 3H), 4.23-4.38 (m, 3H), 6.69-6.99 (s, 1H), 7.12-7.28 (d, 1H), 7.31-7.42 (s, 1H), 7.42-7.51 (m, 1H), 7.68-7.81 (m, 3H), 8.27-8.42 (s, 1H).

### Example 459. Synthesis of Compound 470_P1

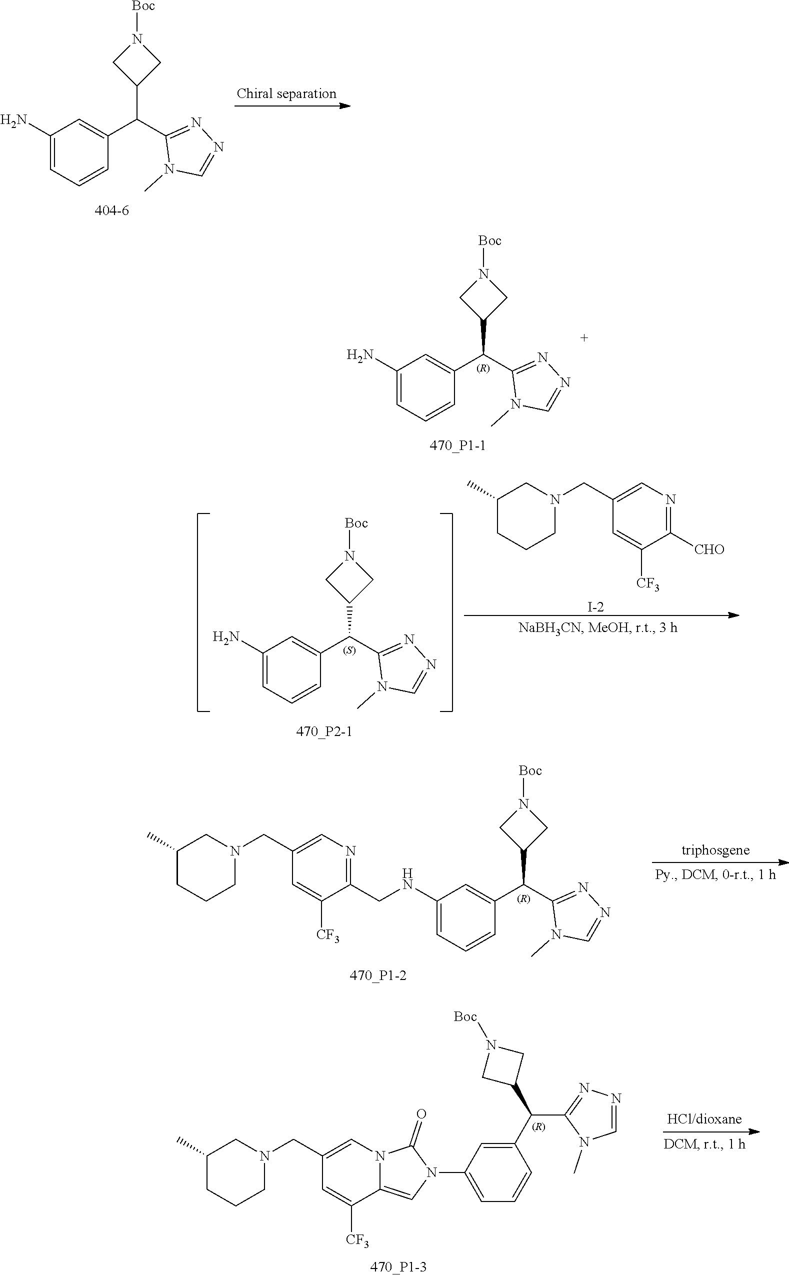

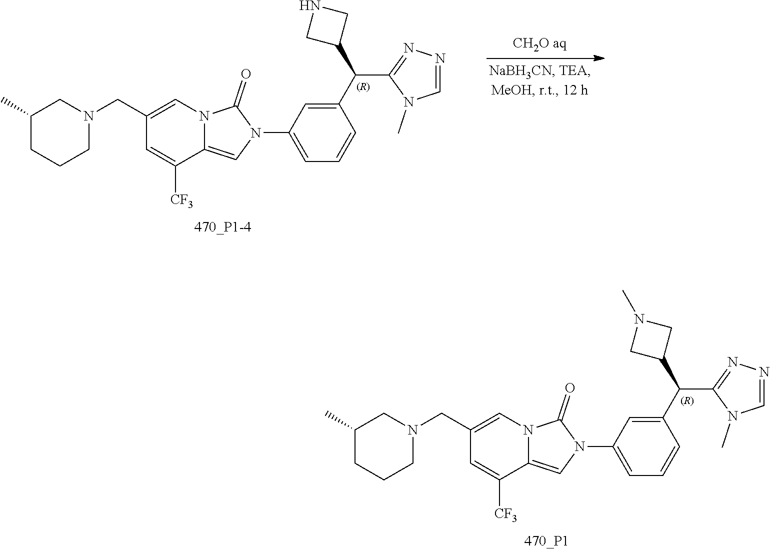

### 1. Synthesis of 470_P1-1

404-6 (1.3 g) was purified by Chiral separation with the following conditions (column: REGIS(S,S)WHELK-O1 (250 nm*50 mm, 10 um); Mobile Phase: [Neu-IPA]; B %: 50%-50%, 6 min, Flow rate: 75 mL/min; Wave Length: 220/254 nm; RT1 (min): 3.24) to afford 470_P1-1 (650 mg, 43%) as a white solid.

### 2. Synthesis of 470_P1-2

To a solution of 470_P1-1 (649 mg, 1.89 mmol, 1 equiv) and 1-2 (541 mg, 1.89 mmol, 1 equiv) in MeOH (15 mL) was added AcOH (324 uL, 5.67 mmol, 3 equiv) The mixture was stirred at 25° C. for 1 h. Then NaBH₃CN (237 mg, 3.78 mmol, 2 equiv) was added, the mixture was stirred at 25° C. for 1 h under nitrogen atmosphere. The reaction mixture was adjusted to pH=8 with sat.NaHCO₃ (30 mL) and extracted with DCM (10 mL×3). The combined organic layers were dried over anhydrous Na₂SO₄, filtered and the filtrate was concentrated under reduced pressure to give a residue. The residue was purified by flash silica gel chromatography eluted with CH₂Cl₂/MeOH=10:1 to afford 470_P1-2 (920 mg, 79%) as a yellow solid.

H-NMR-470_P1-2: (400 MHz, DMSO-d₆) δ 8.68 (s, 1H), 8.31 (s, 1H), 8.02 (s, 1H), 7.01 (t, J=7.6 Hz, 1H), 6.57-6.51 (m, 2H), 6.39 (d, J=7.6 Hz, 1H), 6.21 (t, J=5.6 Hz, 1H), 4.44 (d, J=5.2 Hz, 2H), 4.31 (d, J=10.8 Hz, 1H), 3.95 (s, 1H), 3.62 (d, J=6.4 Hz, 2H), 3.56-3.54 (m, 2H), 3.49 (s, 1H), 3.29 (s, 3H), 2.67 (d, J=6.8 Hz, 2H), 1.93-1.87 (m, 1H), 1.66-1.55 (m, 5H), 1.45 (d, J=12.0 Hz, 1H), 1.36 (s, 9H), 0.90-0.83 (m, 1H), 0.81 (d, J=6.0 Hz, 3H)

### 3. Synthesis of 470_P1-3

To a solution of 470_P1-2 (0.2 g, 325.88 umol, 1 equiv) in DCM (10 mL) was added Py (315 uL, 3.91 mmol, 12 equiv) and bis(trichloromethyl) carbonate (72.5 mg, 244 umol, 0.75 equiv) at 0° C. The mixture was stirred at 25° C. for 0.5 b under nitrogen atmosphere. The same scale reaction was conducted with in parallel for 2 batches in total and work up together. The reaction mixture was quenched with NaHCO₃ (aq. 50 mL). The aqueous layer was extracted with DCM (3×30 mL). The combined organic layers were dried over anhydrous Na₂SO₄, filtered and the filtrate was concentrated under reduced pressure to give a residue. The residue was purified by flash silica gel chromatography eluted with CH₂Cl₂/MeOH=10:1 to afford 470_P1-3 (300 mg, 72%) as a yellow solid.

H-NMR-470_P1-3: (400 MHz, CHLOROFORM-d) δ 7.97 (s, 1H), 7.61-7.48 (m, 3H), 7.38 (t, J=8.0 Hz, 1H), 7.11 (d, J=7.6 Hz, 1H), 6.93 (s, 1H), 6.67 (s, 1H), 4.12 (d, J=10.8 Hz, 1H), 3.67-3.60 (m, 1H), 3.56-3.47 (m, 2H), 3.42 (s, 1H), 3.36 (s, 3H), 3.15 (s, 2H), 2.75-2.64 (m, 2H), 1.84 (t, J=10.0 Hz, 1H), 1.67-1.48 (m, 6H), 1.35 (s, 9H), 0.88-0.81 (m, 1H), 0.79 (d, J=5.6 Hz, 3H)

### 4. Synthesis of 470_P1-4

To a solution of 470_P1-3 (200 mg, 312 umol, 1 equiv) in DCM (3 mL) was added HCl/dioxane (4 M, 1.00 mL, 12.7 equiv). The mixture was stirred at 25° C. for 1 h. The mixture was concentrated under reduced pressure to afford 470_P1-4 (200 mg, crude) as a yellow solid.

### 5. Synthesis of 470_P1

To a solution of 470_P1-4 (200 mg, 370 umol, 1 equiv) in MeOH (2 mL) was added TEA (103 uL, 741.30 umol, 2 equiv) and HCHO (90.2 mg, 1.11 mmol, 37% purity, 3 equiv). The mixture was stirred at 20° C. for 0.5 h. Then NaBH₃Cl (46.6 ng, 741 umol, 2 equiv) was added. The mixture was stirred at 20° C. for 12 h under nitrogen atmosphere. The reaction mixture was adjusted to pH=8 with sat.NaHCO₃ (180 mL) and extracted with DCM (60 mL×3). The combined organic layers were dried over anhydrous Na2SO₄, filtered and the filtrate was concentrated under reduced pressure to give a crude product. The residue was purified by silica gel column chromatography eluted with CH₂Cl₂/MeOH (10:1) to give a crude product. The crude product (160 ng) was purified by prep-HPLC with the following conditions (column: Phenomenex Luna C18 200*40 mm*10 um; Mobile Phase A: Water (0.2% formic acid), Mobile Phase B: ACN; Flow rate: 50 mL/min; Gradient: 1% B to 25% B in 8 min; Wave Length: 220 nm; RT1 (min): 7.5) to afford 470_P1 (74 mg, 11.9%) as a yellow solid.

LCMS-470_P1: (ES, m/z): [M+H]⁺ 554.2

H-NMR-470_P1: (400 MHz, METHANOL-d4) δ 8.56 (s, 1H), 8.45 (s, 1H), 7.77-7.69 (m, 3H), 7.58 (t, J=8.0 Hz, 1H), 7.27 (d, J=7.6 Hz, 1H), 7.17 (d, J=17.6 Hz, 2H), 4.76 (d, J=10.4 Hz, 1H), 4.30 (t, J=8.4 Hz, 1H), 4.14-3.95 (m, 3H), 3.82 (d, J=6.8 Hz, 1H), 3.53 (s, 2H), 3.49 (s, 3H), 3.10-2.95 (m, 2H), 2.89 (s, 3H), 2.25-2.13 (m, 1H), 1.95-1.85 (m, 1H), 1.77 (d, J=3.2 Hz, 3H), 1.71-1.60 (m, 1H), 1.07-0.97 (m, 1H), 0.93 (d, J=6.4 Hz, 3H).

### Example 460. Synthesis of Compound 470_P2

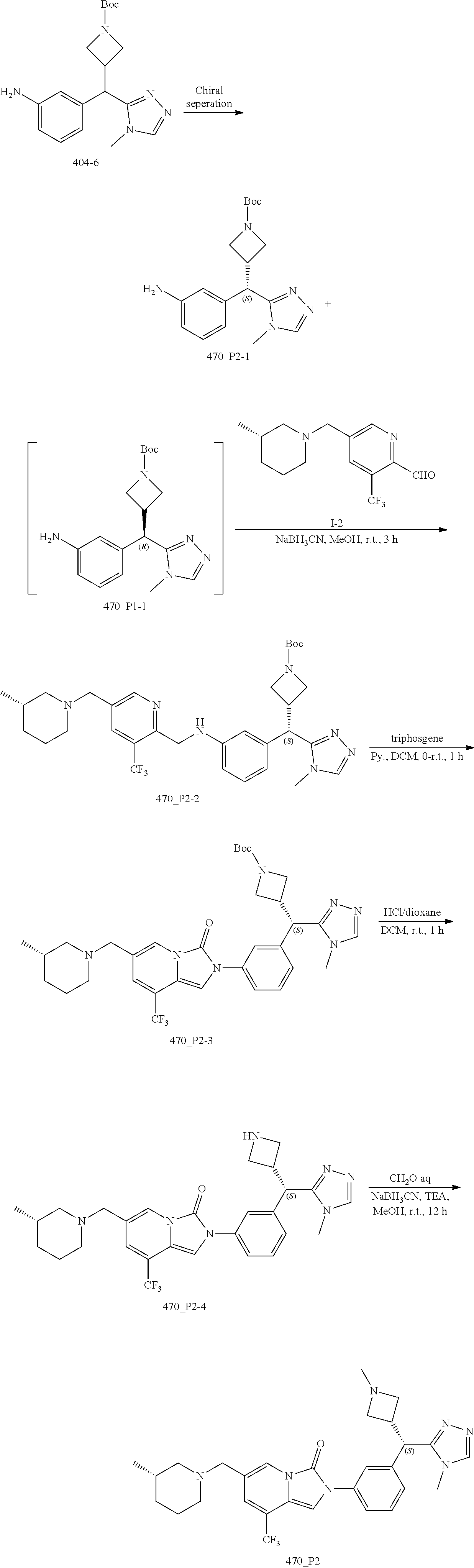

### 1. Synthesis of 470_P2-1

404-6 (1.3 g) was purified by Chiral separation with the following conditions (column: REGIS(S,S)WHELK-O1 (250 mm*50 mm, 10 um); Mobile Phase: [Neu-IPA]; B %: 50%-50%, 6 min, Flow rate: 75 mL/min; Wave Length: 220/254 nm; RT1 (min): 3.60) to afford 470_P2-1 (650 mg, 43%) as a white solid.

### 2. Synthesis of 470_P2-2

To a solution of 1-2 (52191 mg, 1.83 mmol, 1 equiv) and 470_P2-1 (628.46 mg, 1.83 mmol, 1 equiv) in MeOH (7 mL) was added HOAc (314 uL, 5.49 mmol, 3 equiv). The mixture was stirred at 25° C. for 1 hr. And then NaBH₃CN (345 mg, 3.66 mmol, 3 equiv) was added to reaction mixture. The mixture was stirred at 25° C. for 2 h under nitrogen atmosphere. The reaction mixture was adjusted pH to 8 with saturated NaHCO₃ and extracted with CH₂Cl₂ (20 mL×3). The combined organic layers were dried over anhydrous Na2SO4, filtered and concentrated under reduced pressure to give a residue. The residue was purified by flash silica gel chromatography eluted with CH₂Cl₂/MeOH (10:1) to give crude product. The crude product (940 rug) was purified by Prep-HPLC with the following conditions (Column: Waters Xbridge Prep OBD C18 150*40 mm*10 um; Mobile Phase A: Water (10 mmol/L NH₄HCO₃), Mobile Phase B: ACN; Flow rate: 50 mL/min; Gradient: 50% B to 80% B in 8 min; Wave Length: 220 nm; RT11 (min): 8.5) to afford 470_P2-2 (410 mg, 46%) as a white solid.

¹H-NMR-470_P2-2: (400 MHz, METHANOL-d₄) δ 8.68 (s, 1H), 8.35 (s, 1H), 8.12 (s, 1H), 7.12 (t, J=8.0 Hz, 1H), 6.68-6.60 (m, 1H), 6.56-6.50 (m, 2H), 4.54 (s, 2H), 4.33 (d, J=12.0 Hz, 1H), 4.18 (t, J=8.8 Hz, 1H), 3.86-3.73 (m, 2H), 3.64-3.57 (m, 3H), 3.41 (s, 4H), 2.89-2.72 (m, 2H), 1.99 (s, 1H), 1.81-1.52 (m, 6H), 1.44 (s, 9H), 0.97-0.91 (m, 1H), 0.88 (d, J=6.0 Hz, 3H)

### 3. Synthesis of 470_P2-3

To a solution of 470_P2-2 (390 mg, 634 umol, 1 equiv) in CH₂Cl₂ (10 mL) was added Py (308 uL, 3.82 mmol, 6 equiv). And bis(trichloromethyl) carbonate (376 mg, 1270 umol, 2 equiv) was added to reaction mixture at 0° C. The result mixture was stirred at 20° C. for 1 h under nitrogen atmosphere. The reaction mixture was diluted with saturated NaHCO₃ (20 mL), extracted with CH₂Cl₂ (20 mL×3). The combined organic layers were dried over anhydrous Na2SO4, filtered and concentrated in vacuum. The residue was purified by flash silica gel chromatography eluted with CH₂Cl₂/MeOH (10:1) to afford 470_P2-3 (470 mg, 96%) as a yellow solid.

¹H-NMR-470_P2-3: (400 MHz, DMSO-d₆) δ 8.41 (s, 1H), 7.71 (d, J=18.4 Hz, 3H), 7.55 (t, J=8.0 Hz, 1H), 7.31 (d, J=7.6 Hz, 1H), 7.14 (d, J=10.8 Hz, 2H), 4.63 (d, J=12.0 Hz, 1H), 3.94-3.77 (m, 3H), 3.68-3.59 (m, 2H), 3.52 (s, 3H), 3.00-2.76 (m, 3H), 2.06-1.97 (m, 1H), 1.81-1.65 (m, 6H), 1.44 (s, 9H), 0.91 (d, J=5.6 Hz, 4H)

### 4. Synthesis of 470_P2-4

To a solution 470_P2-3 (370 mg, 156 umol, 1 equiv) in DCM (2-5 mL) was added HCl/dioxane (4 M, 235 uL, 6 equiv). The mixture was stirred at 20° C. for 1 h. The reaction mixture was concentrated at room temperature to afford 470_P2-4 (300 ng, crude) as a yellow solid.

### 5. Synthesis of 470_P2

To a solution of 470_P2-4 (78 mg, 145 umol, 1 equiv) and formaldehyde (35.2 ng, 434 mmol, 37% purity, 3 equiv) in MeOH (2 mL) was added TEA (29.3 mg, 289 umol, 2 equiv). The reaction mixture was stirred at 20° C. for 1 h. Then NaBH₃CN (18.2 mg, 289 umol, 2 equiv) was added to reaction mixture. The mixture was stirred at 20° C. for 11 h. The same scale reaction was conducted with in parallel for 3 batches in total and work up with a small test scale (50 mg) together. The reaction mixture was adjusted pH to 8 with saturated NaHCO₃ and extracted with CH₂Cl₂ (20 mL×3). The combined organic layers were dried over anhydrous Na₂SO₄, filtered and concentrated under reduced pressure to give a residue. The residue was purified by flash silica gel chromatography, eluted with CH₂Cl₂/MeOH=10:1 to give crude product. The crude product (450 mg) was purified by Prep-HPLC with the following conditions (Column: Phenomenex Luna C18 200*40 mm*10 um; Mobile Phase A: Water (1.2% formic acid), Mobile Phase B: ACN; Flow rate: 50 mL/min; Gradient: 5% B to 45% B in 8 min; Wave Length: 220 nm; RT1 (min): 9.5) to afford 470_P2 (77 mg, 26%) as a yellow solid.

MS-470_P2: (ES, m/z): [M+H]⁺ 554.2. ¹H-NMR-470_P2: (400 MHz, METHANOL-d₄) δ 8.53 (s, 1H), 8.45 (s, 1H), 7.77 (s, 1H), 7.74-7.70 (m, 2H), 7.59 (t, J=7.6 Hz, 1H), 7.26 (d, J=7.6 Hz, 1H), 7.20 (s, 1H), 7.15 (s, 1H), 4.75 (d, J=10.4 Hz, 1H), 4.41-4.27 (m, 1H), 4.18-3.97 (m, 3H), 3.83 (d, J=9.6 Hz, 1H), 3.57 (s, 2H), 3.48 (s, 3H), 3.14-2.98 (m, 2H), 2.90 (s, 3H), 2.23 (d, J=2.0 Hz, 1H), 1.99-1.89 (m, 1H), 1.84-1.58 (m, 4H), 1.09-0.98 (m, 1H), 0.94 (d, J=6.4 Hz, 3H).

### Example 461. Synthesis of Compound 471

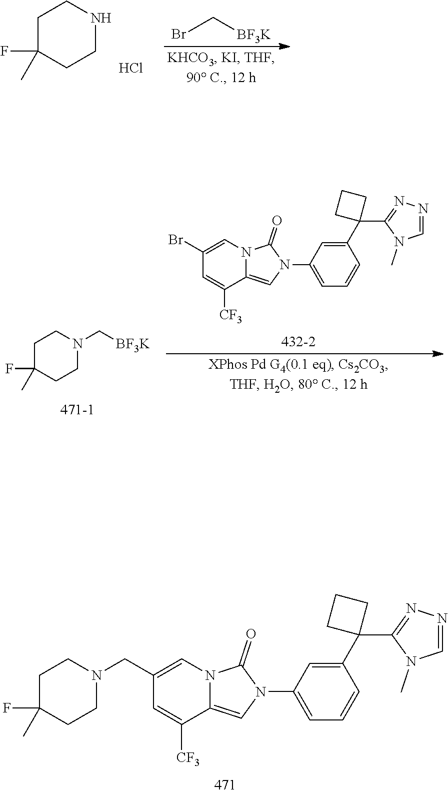

### 1. Synthesis of 471-1

To a solution of 4-fluoro-4-methyl-piperidine (400 mg, 2.6 mmol, 1 equiv, HCl) and potassium; bromomethyl(trifluoro)boranuide (523 mg, 2.6 mmol, 1 equiv) in THF (10 mL) was added KHCO₃ (521 mg, 5.21 mmol, 2 equiv) and KI (43.2 mg, 260 umol, 0.1 equiv). The mixture was stirred at 90° C. for 12 h under nitrogen atmosphere. The mixture was concentrated under reduced pressure. The crude product was triturated with acetone (50 ml) at 35° C. for 1 h. Then the mixture was filtered and the filtrate was concentrated under reduced pressure to afford 471-1 (400 mg, 64%) as a white solid.

### 2. Synthesis of 471

To a solution of 471-1 (241 ng, 1.02 mmol, 2 equiv) and 432-2 (250 mg, 508 umol, 1 equiv) in THY (8 mL) and H2O (2 mL) was added Cs₂CO₃ (496 mg 1.52 mmol, 3 equiv) and dicyclohexyl-[2-(2,4,6-triisopropylphenyl)phenyl]phosphane;methanesulfonate;[2-[2-(methylamino)phenyl] phenyl]palladium(1+) (43.7 mg, 50.8 umol, 0.1 equiv). The mixture was stirred at 80° C. for 12 h under nitrogen atmosphere. The mixture was extracted with water (50 ml) and EtOAc (30 mL×3). The combined organic layers were dried over anhydrous Na2SO₄, filtered and the filtrate was concentrated in vacuum. The residue was purified by silica gel column chromatography eluted with DCM/MeOH (10:1) to give a crude product (0.3 g). The crude product (0.3 g) was purified by Prep-HPLC with the following conditions (column: Phenomenex Luna C18 200*40 mm*10 um; Mobile Phase A: water (FA), Mobile Phase B: ACN; Flow rate: 50 mL/min; Gradient: 1% B to 40% B in 8 min; Wave Length: 220 nm; RT1 (min): 6.2) to afford 471 (120.6 mg, 44%) as a yellow solid.

MS-471: (ES, m/z): [M+H]⁺ 543.1. ¹H-NMR-471: (400 MHz, METHANOL-d4) δ 8.37-8.33 (m, 1H), 7.77-7.71 (m, 2H), 7.62-7.51 (m, 2H), 7.31 (d, J=7.6 Hz, 1H), 7.16 (s, 1H), 7.11 (s, 1H), 3.52 (s, 2H), 3.33 (s, 3H), 3.06-2.97 (m, 2H), 2.87-2.78 (m, 4H), 2.61-2.49 (m, 2H), 2.22-2.06 (m, 2H), 1.94-1.81 (m, 3H), 1.80-1.70 (m, 1H), 1.40-1.32 (m, 3H).

### Example 462. Synthesis of Compound 472_P1

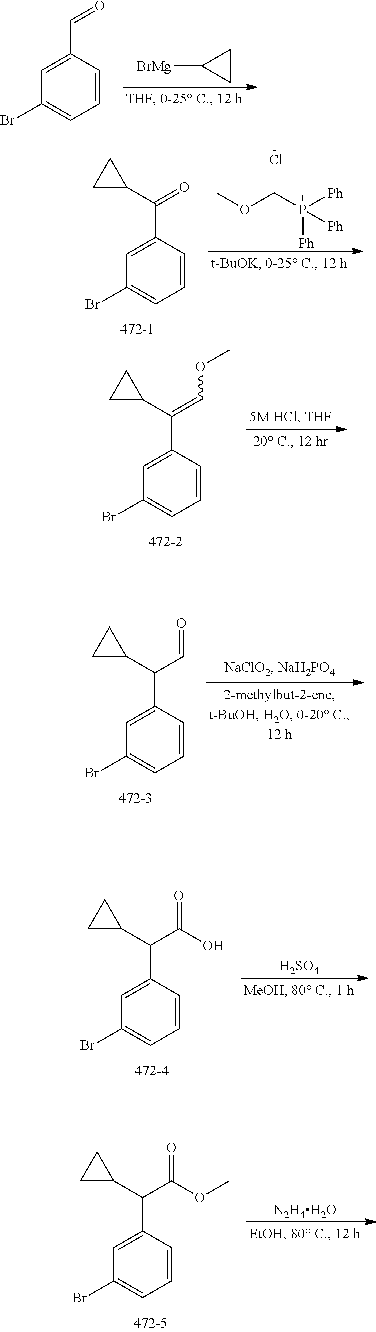

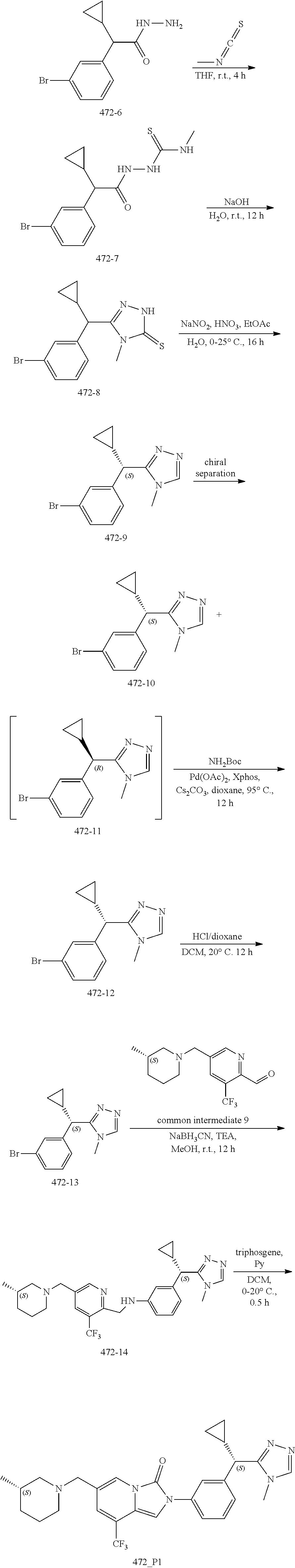

### 1. Synthesis of 472-1

To a solution of 3-bromobenzaldehyde (50.0 g 270 mmol, 31.5 mL, 1.0 equiv) in THF (500.0 mL) at 0° C. After addition, then bromo(cyclopropyl)magnesium (1 M, 351.3 mL, 1.3 equiv) was added dropwise at 0° C. The resulting mixture was stirred at 25° C. for 12 hours. The reaction was poured into water (300.0 mL) and the resulting mixture was extracted with EtOAc (2×150.0 mL). The organic phase was washed with brine (70.0 mL×2), dried over anhydrous Na2SO4, concentrated in vacuum to give a residue. The residue was purified by column chromatography (SiO₂, Petroleum ether/Ethyl acetate=20/1 to 1/1) to afford 472-1 (13.7 g, 22.62% yield) as yellow oil.

### 2. Synthesis of 472-2

To a solution methoxymethyl(triphenyl)phosphonium;chloride (8.4 g, 24.5 mmol, 2.4 equiv) and t-BuOK (2.3 g, 20.4 mmol, 2 equiv) in THF (400.0 mL) was added dropwise DMSO (1.6 g, 20.4 mmol, 1.6 mL, 2.0 equiv) at 0° C. After addition, the mixture was stirred at this temperature for 30 minutes, and then 472-1 (2.3 g, 10.22 mmol, 1.0 equiv) was added at 0° C. The resulting mixture was stirred at 25° C. for 11.5 hours. The reaction was poured into water (150.0 mL) and the resulting mixture was extracted with EtOAc (2×100.0 mL). The organic phase was washed with brine (60.0 mL), dried over anhydrous Na₂SO₄, concentrated in vacuum to give a residue. The residue was purified by column chromatography (SiO₂, Petroleum ether/Ethyl acetate=1/0 to 5/1) to afford 472-2 (2.3 g, 90.21% yield) as colorless oil.

### 3. Synthesis of 472-3

To a solution of 472-2 (12.0 g, 47.4 mmol, 1.0 equiv) in THF (120.0 mL) was added HCl (5 M, 36.0 mL, 3.8 equiv). The mixture was stirred at 20° C. for 12 hours. The reaction was poured into water (100.0 mL) and the resulting mixture was extracted with EtOAc (2×90.0 mL). The organic phase was washed with brine (60.0 mL×2), dried over anhydrous Na2SO4, concentrated in vacuum to give a residue. The crude product was used directly into next step. Compound 472-3 (13.8 g, crude) was obtained as colorless oil.

### 4. Synthesis of 472-4

To a solution of 472-3 (11.5 g, 48.1 mmol, 1.0 equiv) in t-BuOH (180.0 mL) and 2-methylbut-2-ene (90.0 mL) and then sodium;hypochlorite (7.2 g, 96.2 mmol, 5.9 mL, 2.0 equiv) and NaH₂PO₄ (11.5 g, 96.2 mmol, 2.0 equiv) in H2O (20.0 mL) was added dropwise at 0° C. The resulting mixture was stirred at 20° C. for 2 hour. The mixture was added NaHCO₃ (50.0 mL) to adjust pH=8. Then the mixture was partitioned between H₂O (50.0 mL) and EtOAc (100.0 mL). The aqueous phase was separated and added HCl (2M, 40.0 mL) to adjust pH=5, then the aqueous phase was added EtOAc (80.0 mL). The organic phase was separated, washed with brine (40.0 mL), dried over Na₂SO₄, filtered and concentrated under reduced pressure to give a residue. The crude product was used directly into next step. Compound 472-4 (9.8 g, 79.87% yield) was obtained as yellow oil.

### 5. Synthesis of 472-5

To a solution of 472-4 (8.5 g, 33.3 mmol, 1.0 equiv) in MeOH (120.0 mL) was added H₂SO₄ (653 mg, 6.7 mmol, 0.2 equiv). The mixture was stirred at 80° C. for 1 hour. The reaction was poured into water (100.0 mL) and the resulting mixture was extracted with EtOAc (2×80.0 mL). The organic phase was washed with brine (50.0 mL), dried over anhydrous Na₂SO₄, concentrated in vacuum to give a residue. The crude product was used directly into next step. Compound 472-5 (9.0 g, 85.31% yield) was obtained as colorless oil,

H-NMR-472-5: (400 MHz, DMSO-d6) δ ppm 7.44-7.58 (m, 2H), 7.22-7.39 (m, 2H), 3.57-3.69 (m, 3H), 2.93-3.06 (m, 1H), 1.27-1.43 (m, 1H), 0.53-0.67 (m, 1H), 0.27-0.50 (m, 2H), 0.10-0.24 (m, 1H).

### 6. Synthesis of 472-6

To a solution of 472-5 (8.0 g, 29.7 mmol, 1.0 equiv) in EtOH (80.0 mL) was added N₂H₄·H₂O (60.7 g, 1.2 mol, 58.9 mL, 98% purity, 40.0 equiv). The mixture was stirred at 80° C. for 12 hours. The reaction was poured into water (160.0 mL) and the resulting mixture was extracted with EtOAc (2×160.0 mL). The organic phase was washed with brine (20.0 mL), dried over anhydrous Na₂SO, concentrated in vacuum to give a residue. The crude product was used directly into next step. Compound 472-6 (9.0 g, crude) was obtained as colorless oil.

H-NMR-472-6: (400 MHz, DMSO-d₆) δ ppm 9.16 (br s, 1H), 7.52-7.61 (m, 1H), 7.40-7.47 (m, 1H), 7.32-7.38 (m, 1H), 7.22-7.30 (m, 1H), 4.25 (br s, 2H), 2.59 (d, J=10.0 Hz, 1H), 1.32-1.47 (m, 1H), 0.42-0.58 (m, 2H), 0.23-0.34 (m, 1H), 0.01-0.17 (m, 1H).

### 7. Synthesis of 472-7

To a solution of 472-6 (8.0 g, 29.7 mmol, 1.0 equiv) in THF (80.0 mL) was added methylimino(thioxo)methane (4.3 g, 59.4 mmol, 4.0 mL, 2.0 equiv). The mixture was stirred at 20° C. for 4 hours. The reaction was poured to water (60.0 mL) to form solid. The solid was filtered and concentrated. The crude product was used directly into next step. Compound 472-7 (11.5 g, crude) was obtained as a white solid.

### 8. Synthesis of 472-8

To a solution of NaOH (9.8 g, 245.4 mmol, 8.0 equiv) in H₂O (110.0 mL) was added 472-7 (10.5 g, 30.7 mmol, 1.0 equiv). The mixture was stirred at 25° C. for 12 hours. The reaction mixture was added 1M HCl to pH=3-4 to form solid, the solid was filtered and the filter cake was concentrated under reduced pressure. The crude product was used directly into next step. Compound 472-8 (10.0 g, crude) was obtained as a white solid.

### 9. Synthesis of 472-9

HNO₃ (28.6 g, 308.4 mmol, 20.4 mL, 68% purity, 10.0 equiv) was added to H₂O (290.0 ml) to afford the diluted HNO₃ solution (1M, 310.0 ml). To a solution of 472-8 (10.0 g, 30.8 mmol, 1.0 equiv) and NaNO2 (21.3 g, 308.4 mmol, 10.0 equiv) in 112 (100.0 mL) and EtOAc (10.0 mL) was added was added diluted HNO₃ solution (I M, 6.2 mL) dropwise at 0° C. Then the mixture was stirred at 25° C. for 16 hours. The reaction mixture was added a.q. NaHCO₃ to pH=7-8, and the resulting mixture was extracted with EtOAc (2×100.0 mL). The organic phase was washed with brine (80.0 mL), dried over anhydrous Na2SO., concentrated in vacuum to give a residue. The crude product was used directly into next step. Compound 472-9 (9.0 g, 87.89% yield) was obtained as yellow solid.

H-NMR-472-9: (400 MHz, DMSO-d₆) δ ppm 9.16 (br s, 1H), 7.52-7.61 (m, 1H), 7.40-7.47 (m, 1H), 7.32-7.38 (m, 1H), 7.22-7.30 (m, 1H), 4.25 (br s, 2H), 2.59 (d, J=10.0 Hz, 1H), 1.32-1.47 (m, 1H), 0.42-0.58 (m, 2H), 0.23-0.34 (m, 1H), 0.01-0.17 (m, 1H).

### 10. Synthesis of 472-10&11

The 472-9 (4.9 g) was separated by chiral separation with the following condition (column: DAICEL CHIRALPAK IC (250 mm*50 mm, 10 um); mobile phase: [0.1% NH3H2O MEOH]; B %: 50%-50%, 7 min) to afford 472-10 (2.2 g, 43.42% yield) as a yellow solid and 472-11 (26 g, 49.08% yield) as a yellow solid.

H-NMR-472-10: (400 MHz, DMSO-d₆) δ ppm 8.29-8.41 (m, 1H), 7.48-7.52 (m, 1H), 7.42-7.47 (m, 1H), 7.22-7.33 (m, 2H), 3.53-3.63 (m, 1H), 3.36-3.43 (m, 3H), 3.14-3.19 (m, 1H), 1.48-1.66 (m, 1H), 0.45-0.63 (m, 2H), 0.26-0.38 (m, 2H).

H-NMR-472-11: (400 MHz, DMSO-d₆) δ ppm 8.18-8.27 (m, 1H), 7.36-7.40 (m, 1H), 7.28-7.35 (m, 1H), 7.13-7.22 (m, 2H), 3.41-3.51 (m, 1H), 3.24-3.32 (m, 3H), 3.01-3.09 (m, 2H), 1.39-1.51 (m, 1H), 0.34-0.52 (m, 2H), 0.13-0.26 (m, 2H).

### 11. Synthesis of 472-12

A mixture of 472-10 (470 mg, 1.6 mmol, 10 equiv), NH₂BoC (226 mg, 1.9 mmol, 1.2 equiv), Cs₂CO₃ (1.0 g, 3.2 mmol, 2.0 equiv) and Pd(OAc)2 (36 mg, 0.1 equiv), Xantphos (186 mg, 0.2 equiv) in dioxane (10.0 mL) was degassed and purged with N₂ for 3 times, and then the mixture was stirred at 95° C. for 12 hours under N₂ atmosphere. The reaction was poured into water (20.0 mL) and the resulting mixture was extracted with EtOAc (2×10.0 ml). The organic phase was washed with brine (10.0 mL), dried over anhydrous Na₂SO₄, concentrated in vacuum to give a residue. The residue was purified by column chromatography (SiO₂, Dichloromethane: Methanol=50/1 to 5/1) to afford 472-12 (420 mg 79.43% yield) as a yellow solid.

### 12. Synthesis of 472-13

To a solution of 472-12 (420 mg, 1.3 mmol, 1.0 equiv) in DCM (8.0 mL) was added HCl/dioxane (4 M, 1.6 mL, 5.0 equiv). The mixture was stirred at 20° C. for 12 hours. The reaction was concentrated in reduced pressure. The crude product was used directly into next step. Compound 472-13 (450 mg, crude, HCl) was obtained as a yellow solid.

### 13. Synthesis of 472-14

To a solution of 472-13 (210 mg, 1.0 equiv, HCl) and 5-[[(3S)-3-methyl-1-piperidyl]methyl]-3-(trifluoromethyl)pyridine-2-carbaldehyde (272 mg, 1.2 equiv) in MeOH (8.0 mL) was added dropwise TEA (160 mg, 1.6 mmol, 2.0 equiv) at 20° C. After addition, the mixture was stirred at this temperature for 1 hour, and then NaBH₃CN (99 mg, 1.6 mmol, 2.0 equiv) was added dropwise at 20° C. The resulting mixture was stirred at 20° C. for 11 hours. The reaction was poured into water (20.0 mL) and the resulting mixture was extracted with EtOAc (2×10.0 mL). The organic phase was washed with brine (10.0 mL), dried over anhydrous Na₂SO₄, concentrated in vacuum to give a residue. The residue was purified by column chromatography (SiO₂, Dichloromethane: Methanol=30/1 to 1/1) to afford 472-14 (450 mg, 91.03% yield) as yellow oil,

### 14. Synthesis of 472_P1

To a solution of 472-14 (200 mg, 10 equiv) in DCM (4.0 mL) was added dropwise pyridine (190 mg, 2.4 mmol, 6.0 equiv) at 0° C., And then triphosgene (119 ng, 1.0 equiv) in DCM (0.8 mL) was added dropwise at 0° C. The resulting mixture was stirred at 20° C. for 0.5 hour. The reaction was poured into water (20.0 mL) and the resulting mixture was extracted with EtOAc (2×15.0 mL). The organic phase was washed with brine (10.0 mL), dried over anhydrous Na2SO4, concentrated in vacuum to give a residue. The residue was purified by prep-HPLC (column: Phenomenex Luna C18 200*40 mm*10 um; mobile phase: [water (FA)-ACN]; B %: 1%-30%, 8 min) to afford 472-P1 (170 mg, 40.36% yield) as a yellow solid.

MS-472_P1: (ES, m/z): [M+H]+ 525.3.

H-NMR-472_P1: (400 MHz, DMSO-d6) δ ppm 8.30-8.43 (m, 1H), 8.11-8.19 (m, 1H), 7.75 (s, 1H), 7.61-7.73 (m, 2H), 7.46 (t, J=8.0 Hz, 1H), 7.28-7.33 (m, 1H), 7.25 (d, J=7.6 Hz, 1H), 6.93-7.07 (m, 1H), 3.60 (d, J=9.6 Hz, 1H), 3.37-3.49 (m, 3H), 3.22-3.32 (m, 2H), 2.70-2.83 (m, 2H), 1.85-1.99 (m, 1H), 1.53-1.75 (m, 5H), 1.37-1.50 (m, 1H), 0.75-0.94 (m, 4H), 0.56-0.66 (m, 1H), 0.45-0.55 (m, 1H), 0.27-0.42 (m, 2H).

### Example 463. Synthesis of Compound 472_P2

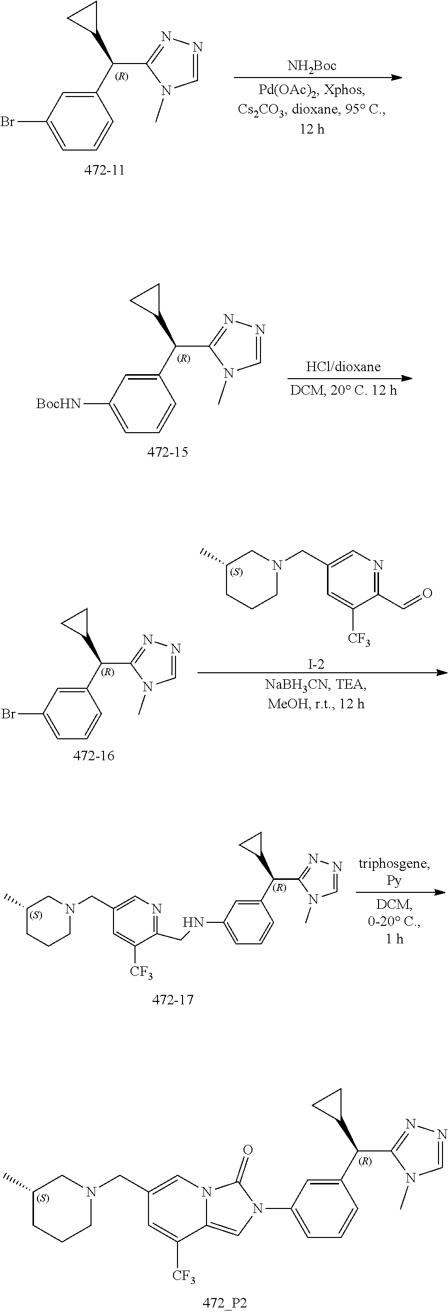

### 1. Synthesis of 472-15

A mixture of 472-11 (619 mg, 2.1 mmol, 10 equiv), NH₂Boc (298 mg, 2.5 mmol, 1.2 equiv), Cs₂CO₃ (1-4 g, 4.2 mmol, 2.0 equiv) and Pd(OAc): (48 mg, 0.1 equiv), Xantphos (245 mg, 0.2 equiv) in dioxane (10 mL) was degassed and purged with N2 for 3 times, and then the mixture was stirred at 95° C. for 12 hours under N₂ atmosphere. The reaction was poured into water (20 mL) and the resulting mixture was extracted with EtOAc (2×10.0 mL). The organic phase was washed with brine (10 mL), dried over anhydrous Na2SO4, concentrated in vacuum to give a residue. The residue was purified by column chromatography (SiO₂, Dichloromethane: Methanol=50/1 to 5/1) to afford 472-15 (470 mg, 67.51% yield) as a yellow solid.

### 2. Synthesis of 472-16

To a solution of 472-15 (470 mg, 1.4 mmol, 1.0 equiv) in DCM (8.0 mL) was added HCl/dioxane (4 M, 1.8 mL, 5.0 equiv). The mixture was stirred at 20° C. for 12 hours. The reaction was concentrated in reduced pressure. The crude product was used directly into next step. Compound 472-16 (420 mg, crude, HCl) was obtained as a yellow solid.

### 3. Synthesis of 472-17

To a solution of 472-16 (210.00 mg, 793.19 umol, 1 equiv, HCl) and 5-[[(3S)-3-methyl-1-piperidyl]methyl]-3-(trifluoromethyl)pyridine-2-carbaldehyde (272.50 mg, 951.83 umol, 1.2 equiv) in MeOH (8 mL) was added dropwise TEA (160.52 mg, 1.59 mmol, 220.80 uL, 2 equiv) at 20° C. After addition, the mixture was stirred at this temperature for 1 hr, and then NaBH3CN (99.69 mg, 1.59 mmol, 2 equiv) was added dropwise at 20° C. The resulting mixture was stirred at 20′C for 11 hr. The residue was purified by column chromatography (SiO₂, Dichloromethane: Methanol=30/1 to 1/1). Compound 472-17 (230 mg, 461.30 umol, 52.66% yield, 80% purity) was obtained as yellow oil.

### 4. Synthesis of 472_P2

To a solution of 472-17 (0.23 g, 461.31 umol, 1 equiv) in DCM (4 mL) was added Py (218.94 mg, 277 mmol, 223.40 uL, 6 equiv). Then a solution of triphosgene (68.45 mg, 230.65 umol, 0.5 equiv) in DCM (2 mL) was added to the above mixture at 0° C. The mixture was stirred at 20° C. for 1 hr under N₂. The same scale reaction was conducted with in parallel for 2 batches in total and work up with a small test scale (50 mg) together. The reaction mixture was quenched by addition water 10 mL at 0° C., and then diluted with DCM 30 mL and extracted with DCM (10 mL*3). The combined organic layers were dried over Na2SO4, filtered and concentrated under reduced pressure to give a residue. The residue was purified by column chromatography (SiO₂, Petroleum ether/Ethyl acetate=10/1 to 0/1) to give part 1: 0.29 g product with 86% LCMS purity; part 2: 0.19 g product with 56% LCMS purity. Two parts of product was further purified by prep-HPLC (FA condition; column: Phenomenex luna C18 80*40 mm*3 um; mobile phase: [water (FA)-ACN]; B %: 5%-40%, 7 min), 179.7 mg 472-P1 (99% purity) was obtained as yellow solid.

MS-472_P2: (ES, m/z): [M+H]+ 525.3.

H-NMR-472_P2: (400 MHz, METHANOL-d4) δ=8.46-8.37 (m, 2H), 7.85 (s, 1H), 7.68 (s, 1H), 7.64 (br d, 1H), 7.51 (t, J=8.0 Hz, 1H), 7.30 (d, J=7.8 Hz, 1H), 7.19 (s, 1H), 7.13 (s, 1H), 3.83 (s, 2H), 3.63 (d, J=9.8 Hz, 1H), 3.53-3.40 (m, 3H), 3.29-3.18 (m, 2H), 2.57-2.46 (m, 1H), 2.24 (s, 1H), 1.90-1.64 (m, 5H), 1.16-1.03 (m, 1H), 0.96 (d, J=6.4 Hz, 3H), 0.80-0.62 (m, 2H), 0.51-0.36 (m, 2H).

### Example 464. Synthesis of Compound 473

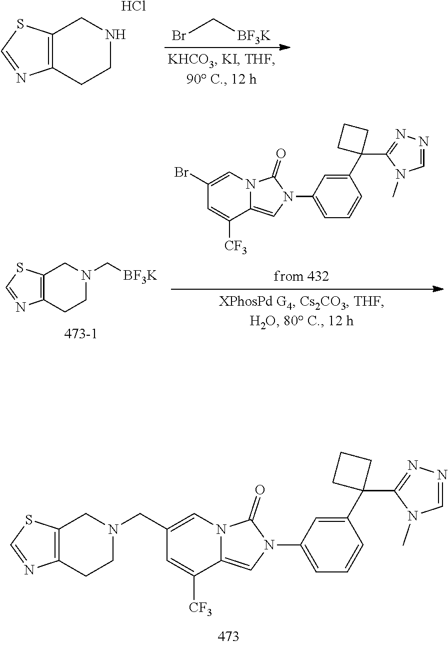

### 1. Synthesis of 473-1

To a solution of 4,5,6,7-tetrahydrothiazolo[5,4-c]pyridine (400 mg, 2.26 mmol, 1 equiv, HCl) in THF (8 mL) was added potassium;hydrogen carbonate (453.35 mg, 4.53 mmol, 2 equiv) and potassium;iodide (37.59 mg, 226.41 umol, 0.1 equiv), potassium;bromomethyl(trifluoro)boranuide (454.72 mg, 2.26 mmol, 1 equiv). The mixture was heated to 90° C. and stirred at 90° C. for 12 hr. The mixture was concentrated under reduced pressure to give a crude. Then the crude was triturated with acetone (60 mL) at 40° C. for 1 h. The mixture was filtered and the filtrate was concentrated under reduced pressure to afford 473-1 (0.27 g, crude) as a light yellow solid,

### 2. Synthesis of 473

To a solution of 473-1 (220 mg, 845.73 umol, 2 equiv) in THF (6 mL) and H₂O (1.5 mL) was added 473-2 (208.17 mg, 422.87 umol, 1 equiv), dicesium;carbonate (413.33 mg, 1.27 mmol, 3 equiv), dicyclohexyl-[2-(2,4,6-triisopropylphenyl)phenyl]phosphane;methanesulfonate;[2-[2 (methylamino)phenyl]phenyl]palladium(1+) (36.39 mg, 42.29 umol, 0.1 equiv). The suspension was degassed and purged with N₂ for 3 times. Then the mixture was heated to 80° C. and stirred at 80° C. for 12 hr under N₂ atmosphere. The reaction mixture was poured into water (10 mL), extracted with EtOAc (15 mL×33). The combined organic layer was washed with brine (30 mL), dried over anhydrous Na₂SO₄, filtered and the filtrate was concentrated in vacuum. The residue was purified by column chromatography (SiO2, Petroleum ether/Ethyl acetate=10/1 to 0/1) and (DCM: Methanol=100/to 0/1) to afford 473 (0.17 g, crude) as a brown solid. The crude product (170 mg) was purified by Prep-HPLC with the following conditions (Column: Phenomenex luna C18 80*40 mm*3 um; mobile phase: [water (FA)-ACN]; B %: 5%-45%, 7 min; Wave Length: 220 nm; RT1 (min): 8) to afford 473 (60.6 mg) as a yellow solid,

MS-473: (ES, m/z): [M+H]+ 566.1. ¹H-NMR-473: (400 MHz, METHANOL-d4) δ 8.85 (s, 1H), 8.35 (s, 1H), 7.77-7.73 (m, 2H), 7.61-7.58 (m, 1H), 7.56-7.52 (m, 1H), 7.31-7.29 (d, J=8 Hz, 1H), 7.14-7.14 (m, 2H), 3.82 (s, 2H), 3.60 (s, 2H), 3.33 (s, 3H), 3.05-2.97 (m, 4H), 2.94-2.93 (m, 2H), 2.85-2.78 (m, 2H), 2.19-2.09 (m, 2H).

### Example 465. Synthesis of Compound 474

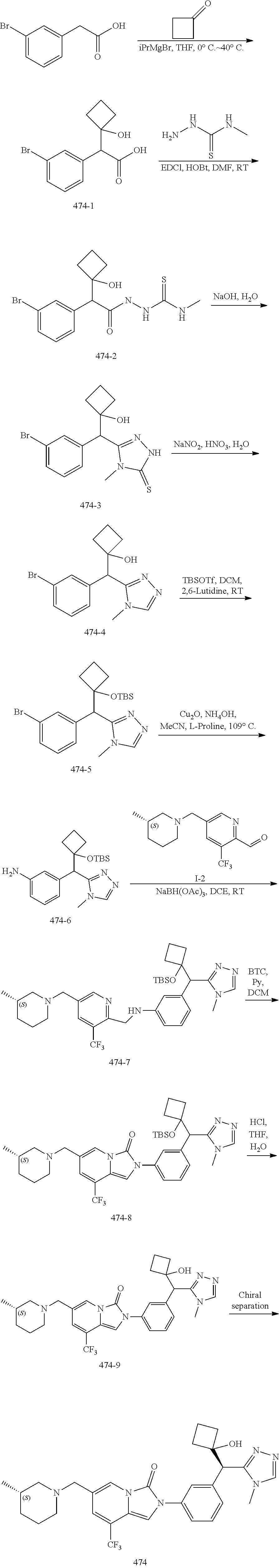

### 1. Synthesis of 474-1

To a stirred solution of M-bromophenylacetic acid (50 g, 232.508 mmol, 1 equiv) in THF (500 mL) was added iPrMgBr (232.5 mL, 465.016 mmol, 2 equiv, 2M) dropwise at 0° C. under nitrogen atmosphere. The resulting mixture was stirred for 1 h at 0° C. under nitrogen atmosphere. To the above mixture was added cyclobutanone (32.59 g, 465,016 mmol, 2 equiv) at room temperature. The resulting mixture was stirred for additional 1 h at 40° C. The reaction was quenched with saturated NH4Cl (aq.) (2000 mL) at room temperature. The aqueous layer was extracted with EtOAc (3×1 L). The resulting mixture was concentrated under reduced pressure to afford 474-1 (40 g, 60.33%) as a yellow oil. The crude product was used in the next step directly without further purification.

### 2. Synthesis of 474-2

To a stirred solution of 474-1 (40 g, 140.283 mmol, 1 equiv) and 1-amino-3-methylthiourea (22.13 g, 210.424 mmol, 1.5 equiv) in DMF (400 mL) were added EDCI (53.78 g, 280.566 mmol, 2 equiv) and HOBt (37.91 g, 280.566 mmol, 2 equiv) at room temperature. The resulting mixture was stirred for overnight at room temperature. The reaction was quenched with saturated NH₄Cl (aq.) (2000 mL) at room temperature. The aqueous layer was extracted with EtOAc (2×1000 mL). The resulting mixture was concentrated under reduced pressure to afford 474-2 (60 g, 114.89%) as a yellow oil. The crude product was used in the next step directly without further purification.

### 3. Synthesis of 474-3

To a stirred mixture of NaOH (16.1 g, 402.530 mmol, 2.50 equiv) in H2O (600 mL) was added 474-2 (60 g, 161.169 mmol, 1 equiv) at room temperature. The resulting mixture was stirred for 4 h at room temperature. The mixture neutralized to pH 7 with 1M HC (aq.). The aqueous layer was extracted with EtOAc (2×600 mL). The resulting mixture was concentrated under reduced pressure to afford 474-3 (22 g, 38.53%) as a yellow solid. The crude product was used in the next step directly without further purification,

### 4. Synthesis of 474-4

To a stirred solution of 474-3 (22 g, 62.100 mmol, 1.00 equiv) and NaNO2 (42.85 g, 621.000 mmol, 10 equiv) was added HNO₃ (41.4 mL, 621,000 mmol, 10 equiv) in H₂O (620 mL) at 0° C. The resulting mixture was stirred overnight at room temperature. The reaction was quenched with NaHCO₃ (aq.) (2000 mL) at room temperature. The aqueous layer was extracted with EtOAc (3×1 L). The resulting mixture was concentrated under reduced pressure. The residue was purified by silica gel column chromatography, eluted with PE/EA=1:1 to afford 474-4 (8 g, 39.98%) as a yellow solid.

### 5. Synthesis of 474-5

To a stirred solution of 474-4 (3 g, 9.311 mmol, 1 equiv) and 2,6-Lutidine (2.00 g, 18.622 mmol, 2 equiv) in DCM (50 mL) were added TBSOTf (3.69 g, 13.966 mmol, 1.5 equiv) at room temperature. The resulting mixture was stirred for 1 h at room temperature. The resulting mixture was diluted with water (200 mL). The aqueous layer was extracted with CH₂Cl₂ (40 mL). The resulting mixture was concentrated under reduced pressure. The residue was purified by silica gel column chromatography, eluted with PE/EA=2:1 to afford 474-5 (3.2 g, 78.74%) as a yellow solid.

### 6. Synthesis of 474-6

To a stirred mixture of 474-5 (3.2 g, 7.332 mmol, 1 equiv) and L-Proline (0.17 g, 1.466 mmol, 0.2 equiv) in MeCN (8 mL) were added NH₄OH (8 mL) and Cu₂O (0.21 g, 1.466 mmol, 0.2 equiv) at room temperature. The resulting mixture was stirred overnight at 100° C. The resulting mixture was filtered, the filter cake was washed with MeOH (10 mL). The filtrate was concentrated under reduced pressure. The residue was purified by Prep-TLC (CH₂Cl₂/MeOH=20:1) to afford 474-6 (1.5 g, 54.91%) as a yellow solid.

### 7. Synthesis of 474-7

To a stirred mixture of 474-6 (900 mg, 2.416 mmol, 1 equiv) and 1-2 (829.88 mg, 2.899 mmol, 1.2 equiv) in DCE (10 mL) were added NaBH(OAc)3 (1535.85 mg, 7.248 mmol, 3 equiv) at room temperature. The resulting mixture was stirred overnight at room temperature. The reaction was quenched with saturated NaHCO₃ (aq.) (80 mL) at room temperature. The aqueous layer was extracted with CH₂Cl₂ (2×50 ml). The resulting mixture was concentrated under reduced pressure. The residue was purified by Prep-TLC (CH₂Cl₂/MeOH=20:1) to afford 474-7 (950 mg, 61.18%) as a yellow solid.

### 8. Synthesis of 474-8

To a stirred solution of 474-7 (960 mg, 1.493 mmol, 1 equiv) and pyridine (708.70 mg, 8.958 mmol, 6 equiv) in DCM (12 mL) were added Triphosgene (155.08 mg, 0.523 mmol, 0.35 equiv) at 0° C. The resulting mixture was stirred for 5 min at 0° C. The reaction was quenched with saturated NaHCO₃ (aq.) (100 mL) at room temperature. The aqueous layer was extracted with CH₂Cl₂ (2×50 mL). The resulting mixture was concentrated under reduced pressure. The residue was purified by Prep-TLC (CH₂Cl₂/MeOH=15:1) to afford 474-8 (800 mg, 80.09%) as a yellow solid.

### 9. Synthesis of 474-9

To a stirred solution of 474-8 (780 mg 1.166 mmol, 1 equiv) in THF (8 mL) were added HCl (8 mL, 2M) at room temperature. The reaction was quenched with saturated NaHCO3 (aq.) (20 mL) at room temperature. The aqueous layer was extracted with EtOAc (2×10 mL). The resulting mixture was concentrated under reduced pressure. The residue was purified by Prep-TLC (CH₂Cl₂/MeOH=15:1) to afford 474-9 (450 mg) as a yellow solid.

### 10. Synthesis of 474

The 474-9 (450 mg) was purified by Prep-Chiral-HPLC with the following conditions (Column: CHIRALPAK IC, 2*25 cm, 5 μm; Mobile Phase A: Hex (05% 2 M NH₃-MeOH), Mobile Phase B: EtOH:DCM=1:1; Flow rate: 20 mL/min; Gradient: 50% B to 50% B in 14.5 min; Wave Length: 220/254 nm; RT1 (min): 8.25; RT2 (min): 11.98; the second peak is product) to afford 474 (186.6 mg, 41.22%) as a yellow solid.

LC-MS-474 (ES, m/z): [M+H]⁺ 555. H-NMR-474 (400 MHz, DMSO-d6, δ ppm): 0.82-0.86 (m, 4H), 1.47-1.50 (m, 1H), 1.50-1.66 (m, 5H), 1.66-1.89 (m, 3H), 1.89-2.14 (m, 2H), 2.14-2.26 (m, 1H), 2.73-2.77 (m, 2H), 3.25-3.30 (m, 2H), 3.45 (s, 3H), 4.56 (s, 1H), 5.50 (s, 1H), 7.01 (s, 1H), 7.24-7.26 (s, 1H), 7.34-7.36 (m, 1H), 7.44-7.46 (m, 1H), 7.65 (s, 1H), 7.65-7.70 (m, 1H), 7.70-7.72 (m, 1H), 7.88 (s, 1H), 8.44 (s, 1H).

### Example 466. Synthesis of Compound 475

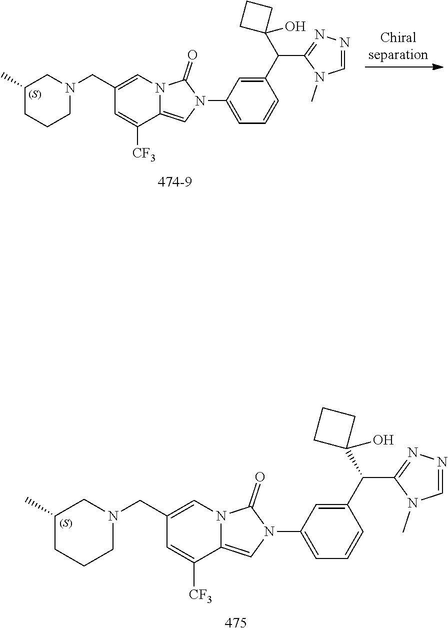

### 1. Synthesis of 475

The 474-9 (450 mg) was purified by Prep-Chiral-HPLC with the following conditions (Column: CHIRALPAK IC, 2*25 cm, 5 μn; Mobile Phase A: Hex (0.5% 2 M NH₃-MeOH), Mobile Phase B: EtOH:DCM=1:1; Flow rate: 20 mL/min; Gradient: 50% B to 50% B in 14.5 min; Wave Length: 220/254 nm; RT1 (min): 8.25; RT2 (min): 11.98; the first peak is product) to afford 475 (175.1 mg, 38.68%) as a yellow solid.

LC-MS-475 (ES, m/z): [M+H]⁺ 555. H-NMR-475 (400 MHz, CD₃OD-d4, δ ppm): 1.73-1.93 (m, 1H), 1.93-1.98 (m, 4H), 2.22-2.29 (m, 1H), 2.62 (s, 2H), 3.08 (s, 3H), 3.23-3.30 (m, 1H), 3.34-3.47 (m, 2H), 4.54 (s, 3H), 3.83 (s, 2H), 4.28-4.31 (m, 1H), 3.83 (s, 2H), 7.11 (s, 1H), 7.15 (s, 1H), 7.27-7.29 (m, 1H), 7.48-7.52 (m, 1H), 7.64-7.66 (m, 1H), 7.82 (s, 1H), 8.38 (s, 1H).

### Example 467. Synthesis of Compound 476

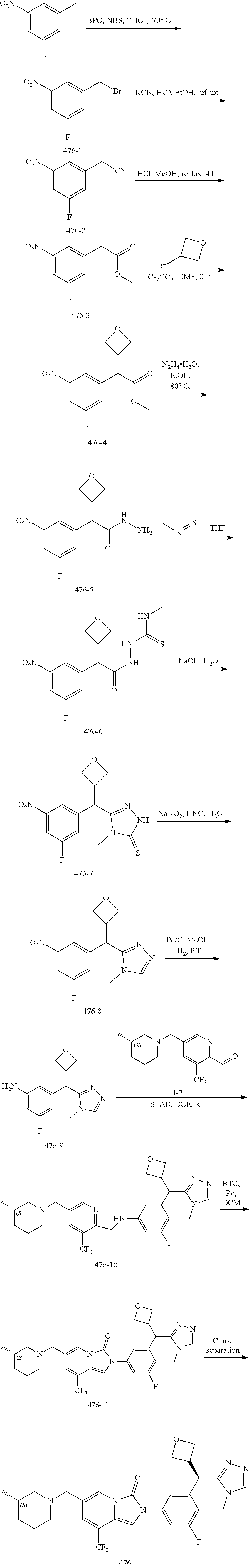

### 1. Synthesis of 476-1

To a stirred mixture of 1-fluoro-3-methyl-5-nitrobenzene (100 g, 644,629 mmol, 1 equiv) and NBS (229.47 g, 1289.258 mmol, 2 equiv) in CHCl₃ (1000 mL) was added BPO (24.78 g, 96.694 mmol, 0.15 equiv) at room temperature under nitrogen atmosphere. The resulting mixture was stirred overnight at 80° C. under nitrogen atmosphere. The reaction was quenched with saturated NH₄Cl (aq.) (500 mL) at room temperature. The resulting mixture was diluted with water (1.5 L). The aqueous layer was extracted with CH2Cl2 (2×1 L) The resulting mixture was concentrated under reduced pressure. The residue was purified by silica gel column chromatography, eluted with PE/EA (50:1) to afford 476-1 (21 g, 13.92%) as a yellow oil.

### 2. Synthesis of 476-2

To a stirred mixture of 476-1 (21 g, 89.734 mmol, 1 equiv) in H₂O (50 mL) and EtOH (200 mL) were added KCN (11.69 g, 179.468 mml, 2 equiv) at room temperature. The resulting mixture was stirred for overnight at 80° C. The reaction was quenched with saturated NaHCO₃ (aq.) (300 mL) at room temperature. The resulting mixture was diluted with water (500 mL). The aqueous layer was extracted with CH₂Cl₂ (2×500 mL). The resulting mixture was filtered, the filter cake was washed with CH₂Cl₂ (2×100 mL). The filtrate was concentrated under reduced pressure. The residue was purified by silica gel column chromatography, eluted with PE/EA (50:1) to afford 476-2 (2.5 g, 15.47%) as a yellow oil.

### 3. Synthesis of 476-3

To a stirred solution of 476-2 (2.5 g, 13.878 mmol, 1 equiv) in MeOH (30 mL) was added HCl (g) over 1 h at room temperature. The resulting mixture was stirred for 2 h at 80° C. The reaction was quenched with NaHCO₃ (aq.) (200 mL) at room temperature. The aqueous layer was extracted with EtOAc (2×80 mL). The resulting mixture was concentrated under reduced pressure. This resulted in 476-3 (2.7 g, 91.27%) as a yellow solid.

### 4. Synthesis of 476-4

To a stirred mixture of methyl 476-3 (2.2 g, 10,321 mmol, 1 equiv) in DMF (40 mL) was added Cs2CO₃ (11.77 g, 36.123 mmol, 3.5 equiv) at 0° C. under nitrogen atmosphere. The resulting mixture was stirred for 3 h at ° C. under nitrogen atmosphere. To the above mixture was added 3-bromooxetane (3.53 g, 25.802 mmol, 2.5 equiv) at room temperature. The resulting mixture was stirred overnight at room temperature. The reaction was quenched with saturated NH₄Cl (aq.) (200 mL) at room temperature. The aqueous layer was extracted with EtOAc (2×100 ml). The resulting mixture was concentrated under reduced pressure. The residue was purified by silica gel column chromatography, eluted with PE/EA (100:1) to afford 476-4 (1.7 g, 61.18%) as a yellow solid.

### 5. Synthesis of 476-5

To a stirred solution of 476-4 (1.6 g, 5,943 mmol, 1 equiv) in EtOH (16 mL) were added NH₂NH₂·H₂O (3.04 g, 59.430 mmol, 10 equiv, 98%) at room temperature. The resulting mixture was stirred overnight at 80° C. The reaction was quenched with saturated NH₄Cl (aq.) (100 mL) at room temperature. The precipitated solids were collected by filtration and washed with water (2×50 mL). This resulted in 476-5 (1.5 g, 93.75%) as a yellow solid.

### 6. Synthesis of 476-6

To a stirred mixture of 476-5 (1.4 g, 5.200 mmol, 1 equiv) in tetrahydrofuran (20 mL) were added methyl isothiocyanate (0.95 g, 13.000 mmol, 2.5 equiv) at room temperature. The resulting mixture was stirred overnight at room temperature. The resulting mixture was diluted with water (20 mL). The resulting mixture was concentrated under reduced pressure. The precipitated solids were collected by filtration and washed with water (2×30 mL). This resulted in 476-6 (1.8 g, 91.00%) as a white solid.

### 7. Synthesis of 476-7

To a stirred mixture of NaOH (2.1 g, 52.580 mmol, 10 equiv) in H₂O (50 mL) was added 476-6 (1.8 g, 5.258 mmol, 1 equiv) at room temperature. The resulting mixture was stirred overnight at room temperature. The mixture was neutralized to pH 7 with 11M HCl (aq.). The aqueous layer was extracted with EtOAc (2×50 mL). The resulting mixture was concentrated under reduced pressure. This resulted in 476-7 (1.2 g, 70.37%) as a yellow solid

### 8. Synthesis of 476-8

To a stirred mixture of 476-7 (1.2 g, 3.700 mmol, 1 equiv) and NaNO₂ (2.55 g, 37.000 mmol, 10 equiv) were added HNO₃ (37 mL, 37.000 mmol, 10 equiv, 1M) at 0° C. The resulting mixture was stirred for 2 h at room temperature. The reaction was quenched with saturated NaHCO₃ (aq.) (100 mL) at room temperature. The aqueous layer was extracted with EtOAc (3×50 mL). The resulting mixture was concentrated under reduced pressure. The residue was purified by Prep-TLC (PE/EA 1:1) to afford 476-8 (700 mg, 64.73%) as a yellow solid.

### 9. Synthesis of 476-9

To a solution of 476-8 (680 mg, 2.327 mmol, 1 equiv) in 20 mL MeOH was added Pd/C (10%, 68 mg) under nitrogen atmosphere in a 100 mL round-bottom flask. The mixture was hydrogenated at room temperature for 2 hi under hydrogen atmosphere using a hydrogen balloon, filtered through a Celite pad and concentrated under reduced pressure. This resulted in 476-9 (620 mg, 101.60%) as a yellow solid.

### 10. Synthesis of 476-10

A solution of 476-9 (600 mg, 2.290 mmol, 1 equiv) and 1-2 (982.41 mg, 3.435 mmol, 1.5 equiv) in DCE (10 mL) was stirred overnight at room temperature. To the above mixture was added STAB (808.04 mg, 3.812 mmol, 2 equiv) at room temperature. The resulting mixture was stirred for additional 4 h at room temperature. The reaction was quenched with saturated NaHCO₃ (aq.) (80 mL) at room temperature. The aqueous layer was extracted with CH₂Cl₂ (3×40 mL). The resulting mixture was concentrated under vacuum. The residue was purified by Prep-TLC (DCM/MeOH 15:1) to afford 476-10 (600 mg, 59.10%) as a white solid.

### 11. Synthesis of 476-11

To a stirred mixture of 476-10 (600 mg, 1.127 mmol, 1 equiv) and Pyridine (534.67 mag, 6.762 mmol, 6 equiv) in DCM (10 mL) was added Triphosgene (133.72 mg, 0.451 mmol, 0.4 equiv) at room temperature. The resulting mixture was stirred for 30 min at room temperature. The reaction was quenched with saturated NaHCO₃ (aq.) (80 mL) at room temperature. The aqueous layer was extracted with DCM (2×30 mL). The resulting mixture was concentrated under vacuum. The residue was purified by Prep-TLC (DCM/MeOH 15:1) to afford 476-11 (300 mg, 47.67%) as a yellow solid.

### 12. Synthesis of 476

The 476-9 (300 mg) was purified by Prep-Chiral-HPLC with the following conditions (Column: CHIRALPAK IG, 2*25 cm, 5 um; Mobile Phase A: Hex (0.5% 2M NH; MeOH), Mobile Phase B: EtOH:DCM=1:1; Flow rate: 20 mL/min; Gradient: 40% B to 40% B in 8.5 min; Wave Length: 220/254 nm; RT1 (min): 6.05; RT2 (min): 6.92; the second peak is product) to afford 476 (84.3 mg, 28.10%) as a yellow solid.

LC-MS-476 (ES, m/z): [M+H]⁺ 559. H-NMR-476 (400 MHz, DMSO-d₆, δ ppm): 0.83-0.91 (m, 4H), 1.43-1.51 (m, 1H), 1.53-1.73 (m, 4H), 1.89-1.95 (m, 1H), 2.74-277 (m, 2H), 3.24-3.29 (m, 2H), 3.42 (s, 3H), 3.92-3.95 (m, 1H), 4.26-4.29 (m, 1H), 4.49-4.50 (m, 2H), 4.72-4.75 (m, 1H), 4.83-4.85 (m, 1H), 7.02-7.06 (m, 2H), 7.46 (s, 1H), 7.64-7.71 (m, 2H), 7.75-7.78 (d, 1H), 8.39 (s, 1H).

### Example 468. Synthesis of Compound 477

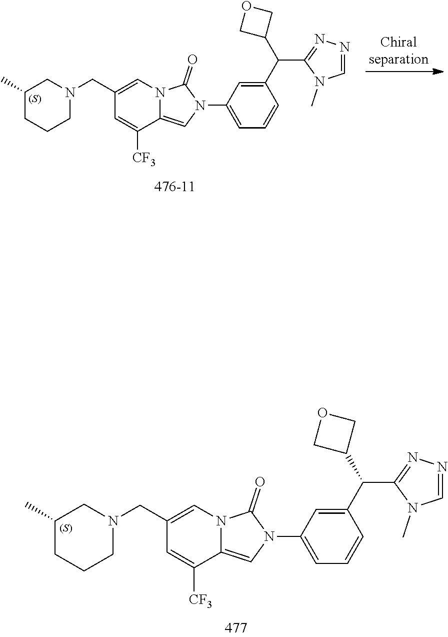

### 1. Synthesis of 477

The 476-11 (300 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 8.5 min; Wave Length: 220/254 nm; RT1 (min): 6.05; RT2 (min): 6.92; the first peak is product) to afford 476 (113.0 mg, 37.66%) as a yellow solid.

LC-MS-477 (ES, m/z): [M+H]⁺ 559. H-NMR-477 (400 MHz, DMSO-d₆, δ ppm): 0.83-0.91 (m, 4H), 1.43-1.51 (m, 1H), 1.53-1.73 (m, 4H), 1.89-1.95 (m, 1H), 2.74-277 (m, 2H), 3.24-3.29 (m, 2H), 3.42 (s, 3H), 3.92-3.95 (m, 1H), 4.26-4.29 (m, 1H), 4.49-4.50 (m, 2H), 4.72-4.75 (m, 1H), 4.83-4.85 (m, 1H), 7.02-7.06 (m, 2H), 7.46 (s, 1H), 7.64 (s, 1H), 7.71 (s, 1H), 7.75-7.78 (d, 1H), 8.39 (s, 1H).

### Example 469. Synthesis of Compound 478

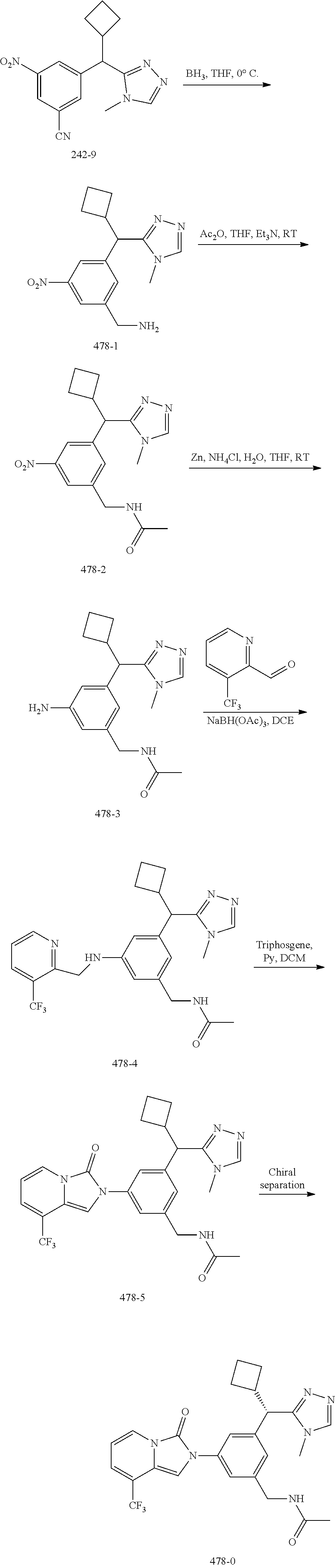

### 1. Synthesis of 478-1

To a stirred solution of 242-9 (2 g, 6.727 mmol, 1 equiv) in THF (20 mL) was added BH3-THF (13.45 mL, 13.454 mmol, 2.00 equiv) dropwise at 0° C. under nitrogen atmosphere. The resulting mixture was stirred for overnight at room temperature under nitrogen atmosphere. The reaction was quenched with HCl (aq.) at room temperature. The mixture was basified to pH 9 with saturated NaHCO₃ (aq.). The aqueous layer was extracted with DCM/MeOH (10:1) (3×30 mL). The resulting mixture was concentrated under vacuum. The residue was purified by Prep-TLC (DCM/MeOH 12:1) to afford 478-1 (600 mg, 27.53%) as an off-white solid.

### 2. Synthesis of 478-2

To a stirred solution of 478-1 (600 mg, 1.991 mmol, 1 equiv) and Et₃N (604.44 mg, 5,973 mmol, 3 equiv) in THF (10 mL) was added Ac₂O (406.53 mg, 3,982 mmol, 2 equiv) at room temperature. The resulting mixture was stirred overnight at room temperature. The resulting mixture was concentrated under vacuum. The residue was purified by Prep-TLC (DCM/MeOH 12:1) to afford 478-2 (550 mg, 72.40%) as an off-white solid.

### 3. Synthesis of 478-3

To a stirred solution of 478-2 (480 mg, 1.398 mmol, 1 equiv) and NH₄Cl (747.70 mg, 13.980 mmol, 10 equiv) in THF (15 mL)/H₂O (5 mL) was added Zn (274.17 mg, 4.194 mmol, 3 equiv) at room temperature. The resulting mixture was stirred for 31 h at room temperature. The resulting mixture was filtered, the filter cake was washed with DCM (2×10 mL). The filtrate was diluted with saturated NaHCO₃ (aq) (100 mL) and extracted with DCM/MeOH (10:1) (3×50 mL). The combined organic layers were concentrated under vacuum. The residue was purified by Prep-TLC (DCM/MeOH 10:1) to afford 478-3 (350 rug, 7430%) as an off-white solid.

### 4. Synthesis of 478-4

To a stirred solution of 478-3 (330 mg, 1.053 mmol, 1 equiv) and 3-(trifluoromethyl)pyridine-2-carbaldehyde (202.82 mg, 1,158 mmol, 1.1 equiv) in DCE (5 mL) was added NaBH(OAc)₃ (446.32 mg, 2.106 mmol, 2 equiv) at room temperature. The resulting mixture was stirred overnight at room temperature. The reaction was quenched with saturated NaHCO₃ (aq.) (60 mL) at room temperature. The aqueous layer was extracted with DCM (3×20 mL). The resulting mixture was concentrated under vacuum. The residue was purified by Prep-TLC (DCM/MeOH 12:1) to afford 478-4 (420 mg, 80.20%) as an off-white solid,

### 5. Synthesis of 478-5

To a stirred solution of 478-4 (400 mg, 0.847 mmol, 1 equiv) and Pyridine (669.61 mg, 8.465 mmol, 10.00 equiv) in DCM (10 mL) were added Triphosgene (10048 ng, 0.339 mmol, 0.4 equiv) at room temperature. The resulting mixture was stirred for 10 min at room temperature. The reaction was quenched with saturated NaHCO₃ (aq.)(60 mL) at room temperature. The aqueous layer was extracted with DCM (2×50 mL). The resulting mixture was concentrated under vacuum. The residue was purified by Prep-TLC (D)CM/MeOH 12:1) to afford 478-5 (300 mg, 68.25%) as a yellow solid.

### 6. Synthesis of 478

The 478-5 (300 mg) was purified by Prep-Chiral-HPLC with the following conditions (Column: CHIRALPAK IH, 2*25 cm, 5 μm; Mobile Phase A: Hex (0.5% 2M NU-MeOH), Mobile Phase B: EtOH:DCM=1:1; Flow rate: 20 mL/min; Gradient: 20% B to 20% B in 12.5 min; Wave Length: 220/254 nm; RT1 (min): 8.37; RT2 (min): 10.19; the first peak is product) to afford 478 (87.0 mg, 27.55%) as a yellow solid.

LC-MS-478 (ES, m/z): [M+H]⁺ 499. H-NMR-478 (400 MHz, DMSO-d6, δ ppm): 1.65-1.76 (m, 1H), 1.76-1.84 (m, 4H), 1.85 (s, 3H), 2.07-2.10 (m, 1H), 3.18-3.25 (m, 1H), 3.43 (s, 3H), 4.23-4.28 (m, 3H), 6.27-6.30 (m, 1H), 7.06-7.11 (m, 2H), 7.26 (s, 1H), 7.58 (s, 1H), 7.64 (s, 1H), 7.77-7.79 (d, 1H), 8.33-8.38 (m, 1H).

### Example 470. Synthesis of Compound 479

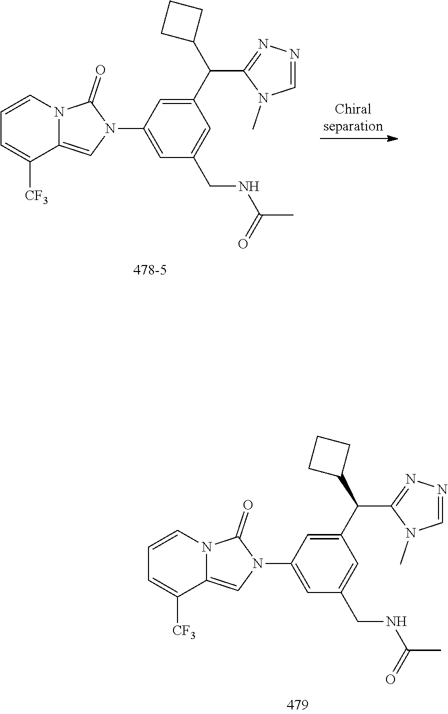 [SMILES: CC(=O)NCc1cc(C(c2nncn2C)C2CCC2)cc(-n2cc3c(C(F)(F)F)cccn3c2=O)c1 ; CC(=O)NCc1cc([C@H](c2nncn2C)C2CCC2)cc(-n2cc3c(C(F)(F)F)cccn3c2=O)c1]

### 1. Synthesis of 479

The 478-5 (300 mg) was purified by Prep-Chiral-HPLC with the following conditions (Column: CHIRALPAK IH, 2*25 cm, 5 Lin; Mobile Phase A: Hex (0.5% 2M N3-MeOH), Mobile Phase B: EtOH:DCM=1:1; Flow rate: 20 mL/min; Gradient: 20% B to 20% B in 12.5 min; Wave Length: 220/254 um; RT1 (min): 8.37; RT2 (min): 10.19; the second peak is product) to afford 479 (87.0 mg, 27.55%) as a yellow solid.

LC-MS-479 (ES, m/z): [M+H]⁺ 499. H-NMR-479 (400 MHz, DMSO-d6, δ ppm): 1.65-1.76 (m, 1H), 1.76-1.84 (m, 4H), 1.85 (s, 3H), 2.07-2.10 (m, 1H), 3.18-3.25 (m, 1H), 3.43 (s, 3H), 4.23-4.26 (m, 3H), 6.27-6.30 (m, 1H), 7.06-7.11 (m, 2H), 7.26 (s, 1H), 7.58 (s, 1H), 7.64 (s, 1H), 7.77-7.79 (d, 1H), 8.33-8.38 (m, 1H).

### Example 471. Synthesis of Compound 480

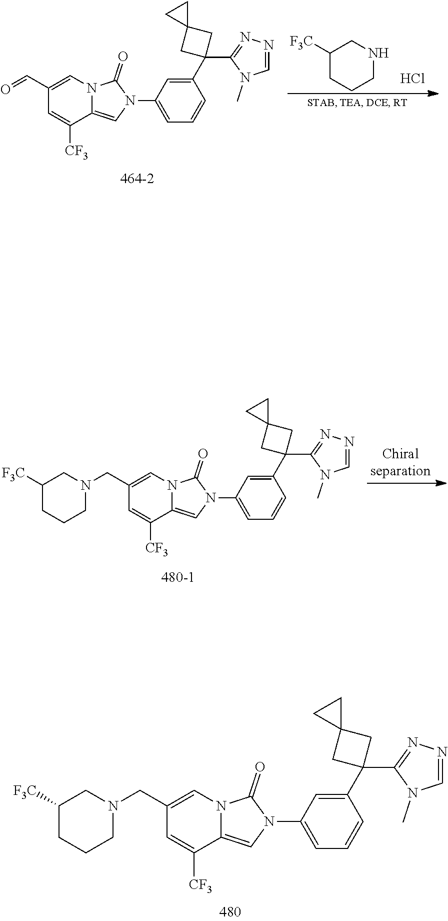 [SMILES: Cn1cnnc1C1(c2cccc(-n3cc4c(C(F)(F)F)cc(C=O)cn4c3=O)c2)CC2(CC2)C1 ; FC(F)(F)C1CCCNC1 ; Cl ; Cn1cnnc1C1(c2cccc(-n3cc4c(C(F)(F)F)cc(CN5CCCC(C(F)(F)F)C5)cn4c3=O)c2)CC2(CC2)C1 ; Cn1cnnc1C1(c2cccc(-n3cc4c(C(F)(F)F)cc(CN5CCC[C@H](C(F)(F)F)C5)cn4c3=O)c2)CC2(CC2)C1]

### 1. Synthesis of 480-1

To a stirred solution of 464-2 (700 mg, 1.497 mmol, 1 equiv) and 3-(trifluoromethyl)piperidine hydrochloride (567.87 mg, 2.994 mmol, 2 equiv) in DCE (10 m L) were added TEA (303.07 mg, 2,994 mmol, 2 equiv) at room temperature. The resulting mixture was stirred for 1 h at room temperature. To the above mixture was added STAB (634.75 mg, 2.994 mmol, 2 equiv) at room temperature. The resulting mixture was stirred for additional overnight at room temperature. The reaction was quenched with saturated NH4C1 (aq.) (80 mL) at room temperature. The aqueous layer was extracted with DCM (2×50 mL). The resulting mixture was concentrated under vacuum. The residue was purified by Prep-TLC (DCM/MeOH 20:1) to afford 480-1 (370 mg, 39.23%) as a yellow solid,

### 2. Synthesis of 480

The 480-1 (370 mg) was purified by Prep-Chiral-HPLC with the following conditions (Column: CHIRALPAK IG, 2*25 cm, 5 μm; Mobile Phase A: Hex (0.5% 2M NH3-MeOH), Mobile Phase B: EtOH:DCM=1:1; Flow rate: 20 mL/min; Gradient: 25% B to 25% B in 15 min; Wave Length: 220/254 nm; RT1 (min): 9.83; RT2 (min): 12.67; the first peak is product) to afford the crude product. The residue was purified by Prep-TLC (DCM/MeOH 20:1) to afford 480 (54.6 mg, 14.05%) as a yellow solid.

LC-MS-480 (ES, m/z): [M+H]⁺ 605. H-NMR-480 (400 MHz, DMSO-d6, δ ppm): 0.43-0.49 (m, 2H), 0.55-0.59 (m, 2H), 1.24-1.34 (m, 1H), 1.47-1.58 (m, 1H), 1.83-1.91 (m, 1H), 1.97-2.05 (m, 2H), 2.51-2.61 (m, 1H), 2.75-2.80 (m, 3H), 2.97-3.03 (m, 1H), 3.17-3.24 (m, 2H), 3.32 (s, 3H), 3.40 (s, 2H), 7.02 (s, 1H), 7.18-7.20 (d, 1H), 7.35 (s, 1H), 7.49-7.53 (m, 1H), 7.63-7.65 (d, 1H), 7.72 (s, 1H), 7.83 (s, 1H), 8.39 (s, 1H).

### Example 472. Synthesis of Compound 481

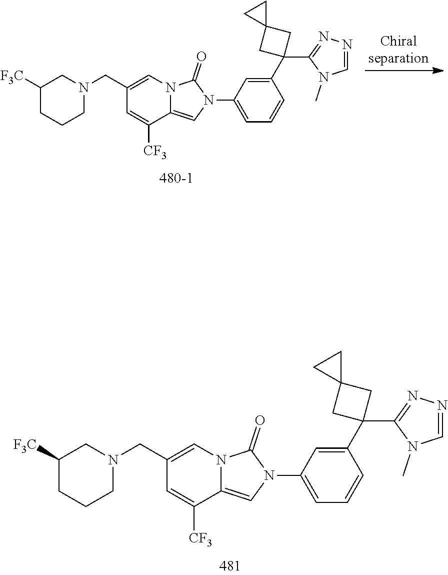 [SMILES: Cn1cnnc1C1(c2cccc(-n3cc4c(C(F)(F)F)cc(CN5CCCC(C(F)(F)F)C5)cn4c3=O)c2)CC2(CC2)C1 ; Cn1cnnc1C1(c2cccc(-n3cc4c(C(F)(F)F)cc(CN5CCC[C@@H](C(F)(F)F)C5)cn4c3=O)c2)CC2(CC2)C1]

### Synthesis of 481

480-1 (370 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 25% B to 25% B in 15 min; Wave Length: 220/254 nm; RT1 (min): 9.83; RT2 (min): 12.67; the second peak is product) to afford 481 (145.8 mg, 38.85%) as a yellow solid.

LC-MS-481 (ES, m/z): [M+H]⁺ 605. H-NMR-481 (400 MHz, DMSO-d6, δ ppm): 0.43-0.49 (m, 2H), 0.55-0.59 (m, 2H), 1.24-1.34 (m, 1H), 1.47-1.58 (m, 1H), 1.83-1.91 (m, 1H), 1.97-2.05 (m, 2H), 2.51-2.61 (m, 1H), 2.75-2.80 (m, 3H), 2.97-3.03 (m, 1H), 3.17-3.24 (m, 2H), 3.32 (s, 3H), 3.40 (s, 2H), 7.02 (s, 1H), 7.17-7.19 (d, 1H), 7.35 (s, 1H), 7.49-7.52 (m, 1H), 7.63-7.65 (d, 1H), 7.72 (s, 1H), 7.83 (s, 1H), 8.39 (s, 1W).

### Example 473. Synthesis of Compound 482

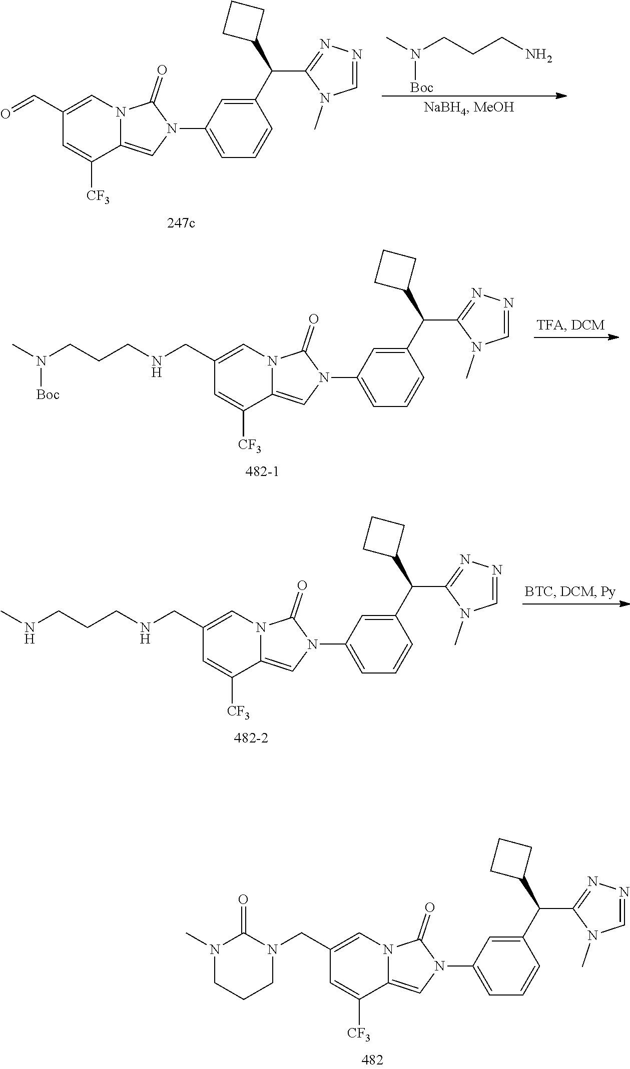

### 1. Synthesis of 482-1

To a stirred solution of 247-3 (800 mg, 1.757 mmol, 1 equiv) in MeOH (7 ML) was added tert-butyl N-(3-aminopropyl)-N-methylcarbamate (661.41 mg, 3.514 mmol, 2 equiv) was stirred for overnight at room temperature. To the above mixture was added NaBH₄ (332.25 mg, 8.785 mmol, 5 equiv). The resulting mixture was stirred for additional 1 h at room temperature. The reaction was quenched with sat, NH₄Cl (20 mL) at room temperature. The aqueous layer was extracted with CH₂Cl₂ (3×10 mL). The resulting mixture was concentrated under reduced pressure. The residue was purified by Prep-TLC (DCM/MeOH 10:1) to afford 482-1 (380 mg, 27.57%) as a brown oil.

### 2. Synthesis of 482-2

A solution of tert-butyl 482-1 (380 mg, 0.605 mmol, 1 equiv) and TFA (3 mL) in DCM (9 mL) was stirred for 3 h at room temperature. The reaction was quenched with sat. NaHCO₃ (aq.) (50 mL) at room temperature. The aqueous layer was extracted with Cl2Cl₂ (3×20 mL). The resulting mixture was concentrated under reduced pressure. The residue was purified by Prep-TLC (CH₂Cl₂/MeOH 8:1) to afford 482-2 (100 mg, 29.12%) as a brown oil.

### 3. Synthesis of 482

To a stirred solution of 482-2 (100 mg, 0,152 mmol, 1 equiv) and pyridine (71.96 mg, 0.912 mmol, 6 equiv) in DCM (10 mL) were added triphosgene (18.00 mg, 0.061 mmol, 0.4 equiv) at 0° C. The resulting mixture was stirred for 5 min at room temperature. The reaction was quenched with sat. NaHCO₃ (aq.) (50 mL) at room temperature. The aqueous layer was extracted with EtOAc (3×20 mL). The resulting mixture was concentrated under reduced pressure. The residue was purified by Prep-TLC (CH₂Cl₂/MeOH 8:1) to afford crude product. The crude product (60 mg) was purified by Prep-HPLC with the following conditions (Column: X Bridge Shield RP18 OBD Column, 30*150 mm, 5 Lm; Mobile Phase A: water (10 mmol/L NH₄HCO₃), Mobile Phase B: ACN; Flow rate: 60 mL/min; Gradient: 27% B to 47% B in 8 min; Wave Length: 220 nm; RT1 (min): 7.37) to afford 482 (21.0 mg, 24.84%) as a light yellow solid.

LC-MS-482 (ES, m/z): [M+H]⁺ 554. H-NMR-482 (400 MHz, CD₃OD, ppm): 51.75-1.80 (m, 1H), 51.86-1.92 (m, 4H), 51.93-2.04 (m, 2H), 62.24-2.28 (m, 1H), 62.97 (s, 3H), 63.14-3.16 (m, 1H), 63.32-3.38 (m, 4H), 63.35-3.56 (m, 3H), 64.28-4.36 (d, 1H), 64.87 (s, 2H), 67.04-7.14 (d, 1H), 67.26-7.29 (d, 1H), 67.48-7.50 (d, 1H), 67.52 (m, 1H), 67.63-7.67 (m, 3H), 68.38 (s, 1H).

### Example 474. Synthesis of Compound 483

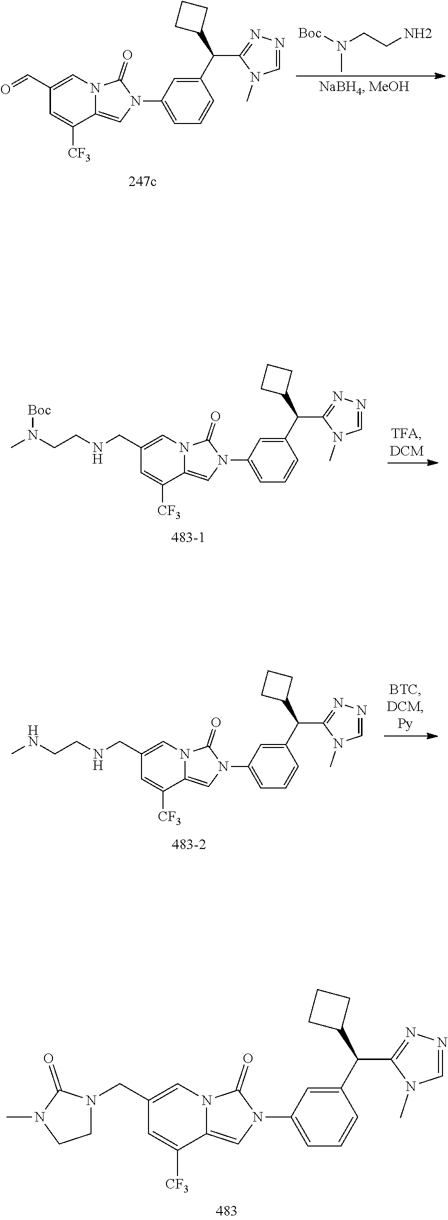 [SMILES: Cn1cnnc1[C@@H](c1cccc(-n2cc3c(C(F)(F)F)cc(C=O)cn3c2=O)c1)C1CCC1 ; CN(CCN)C(=O)OC(C)(C)C ; CN(CCNCc1cc(C(F)(F)F)c2cn(-c3cccc([C@H](c4nncn4C)C4CCC4)c3)c(=O)n2c1)C(=O)OC(C)(C)C ; CNCCNCc1cc(C(F)(F)F)c2cn(-c3cccc([C@H](c4nncn4C)C4CCC4)c3)c(=O)n2c1 ; CN1CCN(Cc2cc(C(F)(F)F)c3cn(-c4cccc([C@H](c5nncn5C)C5CCC5)c4)c(=O)n3c2)C1=O]

### 1. Synthesis of 483-1

To a stirred solution of 247c (800 mg, 1.757 mmol, 1 equiv) in MeOH (7 mL) was added tert-butyl N-(2-aminoethyl)-N-methylcarbamate (612.13 mg, 3.514 mmol, 2 equiv) was stirred for overnight at room temperature. To the above mixture was added NaBH₄. (33225 mg, 8.785 mmol, 5 equiv) The resulting mixture was stirred for additional 1 h at room temperature. The reaction was quenched with sat. NH₄Cl (20 mL) at room temperature. The aqueous layer was extracted with CH2Cl2 (3×10 mL). The resulting mixture was concentrated under reduced pressure. The residue was purified by Prep-TLC (CH₂Cl₂/MeOH 10:1) to afford 483-1 (330 mg, 26.94%) as a brown oil.

### 2. Synthesis of 483-2

A solution of 483-1 (330 mg, 0,538 mmol, 1 equiv) and TFA (2 mL) in DCM (6 mL) was stirred for 3 h at room temperature. The reaction was quenched with sat. NaHCO₃ (aq.) (50 mL) at room temperature. The aqueous layer was extracted with CH₂Cl₂ (3×20 mL). The resulting mixture was concentrated under reduced pressure. The residue was purified by Prep-TLC (CH₂Cl₂/MeOH 8:1) to afford 483-2 (120 mg, 34.76%) as a brown oil.

### 3. Synthesis of 483

To a stirred solution of 483-2 (120 mg, 0.234 mmol, 1 equiv) and pyridine (110.89 mg, 1.404 mmol, 6 equiv) in DCM (10 mL) was added triphosgene (2773 mg, 0.094 mmol, 0.4 equiv) at 0° C. The resulting mixture was stirred for 5 min at room temperature. The reaction was quenched with sat. NaHCO₃ (aq.) (50 mL) at room temperature. The aqueous layer was extracted with EtOAc (3×30 mL). The resulting mixture was concentrated under reduced pressure. The residue was purified by Prep-TLC (DCM/MeOH 8:1) to afford the crude product (80 mg). The crude product (80 mg) was purified by Prep-HPLC with the following conditions (Column: X Bridge Shield RP18 OBD Column, 30*150 mm, 5 μm; Mobile Phase A: water (10 mmol/L NH₄HCO₃), Mobile Phase B: ACN; Flow rate: 60 mL/min; Gradient: 26% B to 46% B in 8 min; Wave Length: 220 nm; RT1 (min): 7.35) to afford 483 (41.9 mg, 32.95%) as a light yellow solid.

LC-MS-483 (ES, m/z): [M+H]⁺ 541. H-NMR-483 (400 MHz, CD₃OD, ppm): δ 1.75-1.81 (m, 1H), 51.86-1.97 (m, 4H), 52.24-2.28 (m, 1H), 32.84 (s, 3H), 33.15 (s, 1H), 33.27-3.34 (m, 4H), 33.34-3.56 (m, 3H), 34.19 (s, 2H), 34.28-4.31 (d, 1H), 36.98 (s, 1H), 37.14 (s, 1H), 37.26-7.28 (d, 1H), 37.29 (s, 1H), 37.48-7.52 (m, 2H), 37.63-7.71 (m, 1H), 38.38 (s, 1H).

### Example 475. Synthesis of Compound 484

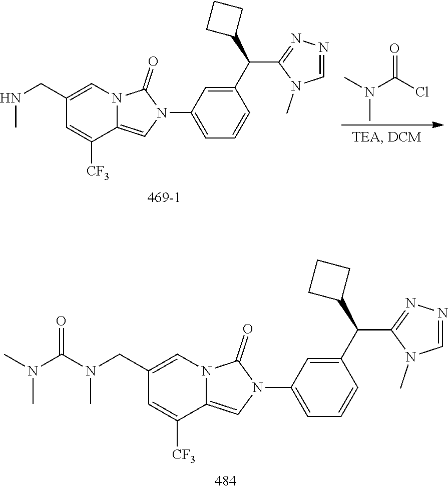

### 1. Synthesis of 484

Into a 20 mL sealed tube were added 469-1 (160 mg, 0.340 mmol, 1 equiv), DCM (3 mL), TEA (103.24 mg, 1,020 mmol, 3 equiv) and dimethylcarbamyl chloride (73.14 mg, 0.680 mmol, 2 equiv) at room temperature. The final reaction mixture was irradiated with microwave radiation for 2 h at room temperature. The reaction was quenched with water (10 mL) at room temperature. The aqueous layer was extracted with CH₂Cl₂ (3×5 mL). The resulting mixture was concentrated under reduced pressure. The residue was purified by Prep-TLC (CH₂Cl₂/MeOH 15:1). The crude product (100 mg) was purified by Prep-HPLC with the following conditions (Column: XBridge Shield RP18 OBD Column, 30*150 nm, 5 μm; Mobile Phase A: Water (10 mmol/L. NH4HCO₃), Mobile Phase B: ACN; Flow rate: 60 mL/min; Gradient: 28% B to 48% B in 8 min; Wave Length: 220 nm; RT1 (min): 7.33) to afford 484 (59.4 mg, 31.90%) as a yellow solid.

LC-MS-484 (ES, m/z): [M+H]⁺ 542. H-NMR-484 (400 MHz, DMSO, δ ppm): 1.52-1.90 (m, 5H), 1.99-2.18 (s, 1H), 2.64-2.75 (s, 3H), 2.75-2.80 (s, 6H), 3.15-3.30 (m, 1H), 3.39-3.51 (s, 3H), 3.91-4.21 (s, 2H), 4.21-4.35 (d, 1H), 6.80-7.12 (s, 1H), 7.12-7.28 (d, 1H), 7.28-7.40 (d, 1H), 7.40-7.65 (m, 1H), 7.65-7.80 (m, 3H), 8.21-8.40 (s, 1H).

### Example 476. Synthesis of Compound 485

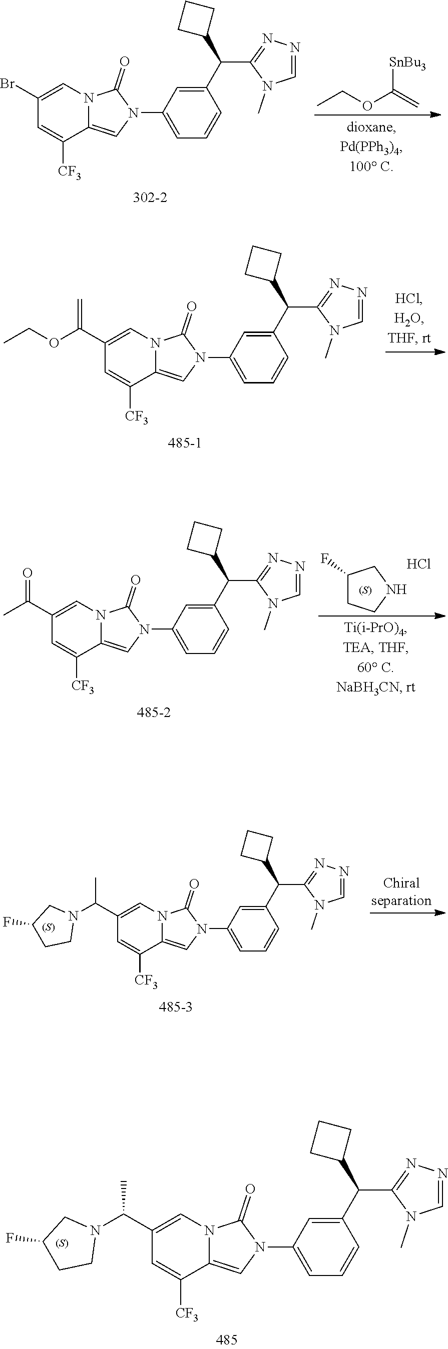

### 1. Synthesis of 485-1

Into a 250 mL 3-necked round-bottom flask were added 302-2 (10 g, 19.750 mmol, 1 equiv), tributyl(1-ethoxyethenyl) stannane (10.70 g, 29.625 mmol, 1.5 equiv), Pd(PPh3)4 (2.28 g, 1.975 mmol, 0.1 equiv) and dioxane (100 mL) at room temperature. The resulting mixture was stirred for overnight at 100° C. under nitrogen atmosphere. The resulting mixture was diluted with water (500 mL). The aqueous layer was extracted with EtOAc (3×200 mL). The resulting mixture was concentrated under reduced pressure. The residue was purified by silica gel column chromatography, eluted with PE/EA (1:1) to afford 485-1 (8 g, 76.53%) as a yellow solid.

### 2. Synthesis of 485-2

Into a 250 mL round-bottom flask were added 485-1 (8 g, 16.080 mmol, 1 equiv), THF (20 mL) and 1M HCl (20 mL) at room temperature. The resulting mixture was stirred for overnight at room temperature. The resulting mixture was diluted with water (50 mL), The aqueous layer was extracted with EtOAc (3×30 mL). The resulting mixture was concentrated under reduced pressure. The residue was purified by silica gel column chromatography, elated with CH₂Cl₂/MeOH (100:1) to afford 485-2 (7 g, 86.24%) as a yellow solid.

### 3. Synthesis of 485-3

Into a 40 mL vial were added 485-2 (500 mg, 1.065 mmol, 1 equiv), (3S)-3-fluoropyrrolidine hydrochloride (160.49 mg, 1278 mmol, 1.2 equiv), TEA (431.10 mg, 4.260 mmol, 4 equiv), Ti(Oi-Pr)₄ (605.41 mg, 2.130 mmol, 2 equiv) and THF (10 mL) at room temperature. The mixture was stirred for 1 h at room temperature. To the above mixture was added NaBH₃CN (133.85 mg, 2.130 mmol, 2 equiv) at room temperature. The resulting mixture was stirred for overnight at 60° C. The resulting mixture was diluted with water (100 mL). The aqueous layer was extracted with EtOAc (3×50 mL). The resulting mixture was concentrated under reduced pressure. The residue was purified by Prep-TLC (CH₂Cl₂/MeOH 15:1) to afford crude product. The crude product (250 mg) was purified by Prep-HPLC with the following conditions (Column: X Bridge Prep OBD C18 Column, 30*150 mm, 5 μm; Mobile Phase A: Water (10 mmol/L NH₄HCO₃), Mobile Phase B: ACN; Flow rate: 60 ml/min; Gradient: 40% B to 53% B in 8 min; Wave Length: 254 am; RT1 (min): 7.37) to afford 485-3 (120 mg, 20.60%) as a yellow solid.

### 4. Synthesis of 485

The 485-3 (150 rug, 0.276 mmol, 1 equiv) was purified by Chiral seperation with the following conditions (Column: CHIRALPAK IG, 7*25 cm, 10 μm; Mobile Phase A: Hex (0.5% 2M NH₃-MeOH)-HPLC, Mobile Phase B: EtOH:DCM=1:1-HPLC; Flow rate: 20 mL/min; Gradient: 15% B to 15% B in 19 min; Wave Length: 220/254 nm; RT1 (min): 12.72; RT2 (min): 15.97; The first peak was the product. Sample Solvent: EtOH:DCM=1:1-HPLC; Injection Volume: 0.7 mL; Number of Runs: 8) to afford 485 (42.4 mg, 28.01%) as a yellow solid.

LC-MS-485 (ES, m/z): [M+H]⁺ 543. H-NMR-485 (400 MHz, DMSO-d6, ppm): δ 1.30 (s, 3H), 31.60-1.97 (m, 6H), 32.05-2.12 (m, 2H), 52.40-2.50 (m, 1H), 52.65-2.67 (m, 2H), 32.80-2.95 (m, 1H), 33.20-3.28 (m, 2H), 33.43 (s, 3H), 54.25-4.28 (d, 1H), 35.13-5.28 (d, 1H), 37.05 (s, 1H), 37.19-7.21 (d, 1H), 37.31 (s, 1H), 37.42-7.46 (m, 1H), 37.67-7.70 (m, 311), 38.33 (s, 1H).

### Example 477. Synthesis of Compound 486

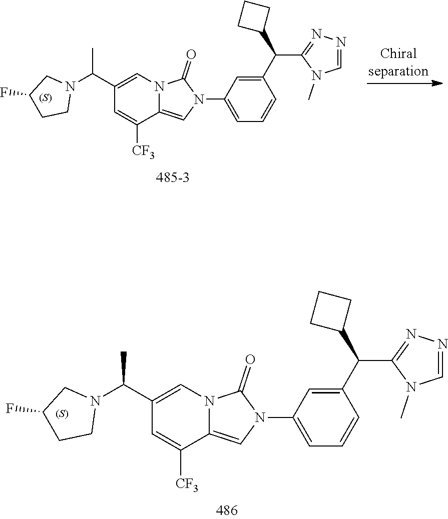

### 1. Synthesis of 486

The 485-3 (150 mg 0,276 mmol, 1 equiv) was purified by Chiral separation with the following conditions (Column: CHIRALPAK IG, 7*25 cm, 10 μm; Mobile Phase A: Hex (0.5% 2M NH₃-MeOH)-HPLC, Mobile Phase B: EtOH:DCM=1:1-HPLC; Flow rate: 20 mL/min; Gradient: 15% B to 15% B in 19 min; Wave Length: 220/254 nm; RT1 (min): 12.72; RT2 (min): 15.97; The second peak was the product. Sample Solvent: EtOH:DCM=1:1-HPLC; Injection Volume: 0.7 mL; Number of Runs: 8) to afford 486 (40.4 mg, 26.69%) as a yellow solid.

LC-MS-485 (ES, m/z): [M+H]⁺ 543. H-NMR-485 (400 MHz, DMSO-d6, ppm): δ 1.28-1.30 (d, 3H), 51.77-1.81 (m, 6H), 52.08-2.29 (m, 3H), 52.65-2.67 (m, 2H), 32.80-2.95 (m, 1H), 33.20-3.28 (m, 2H), 33.43 (s, 3H), 34.25-4.27 (d, 1H), 35.11-5.25 (d, 1H), 37.06 (s, 1H), 37.19-7.21 (d, 1H), 37.31 (s, 1H), 37.42-7.46 (m, 1H), 37.67-7.72 (m, 3H), 38.33 (s, 1H).

### Example 478. Synthesis of Compound 487

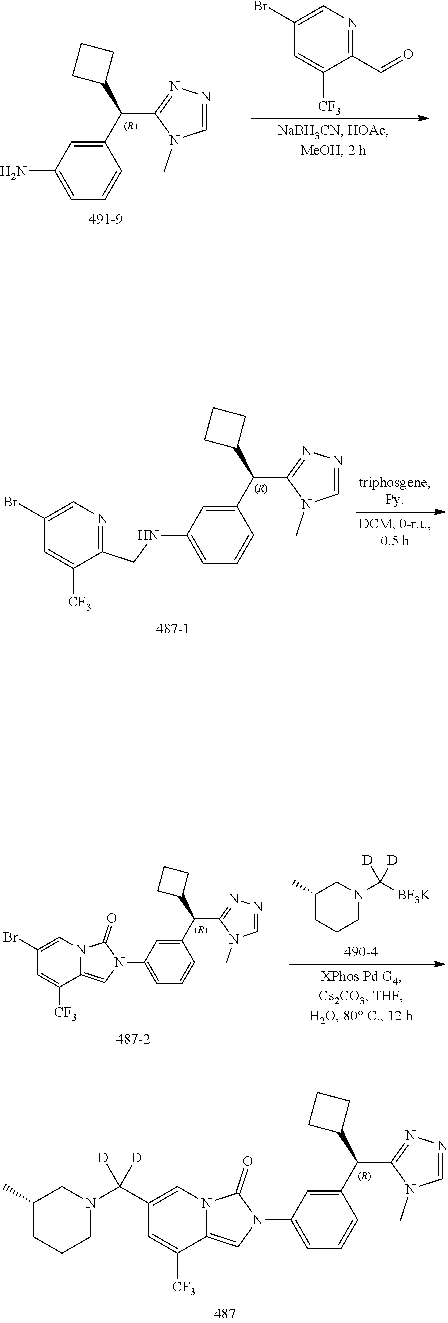

### 1. Synthesis of 487-1

To a solution of 491-9 (3.9 g, 16.1 mmol, 1 equiv) and 5-bromo-3-(trifluoromethyl)picolinaldehyde (4.09 g, 16.1 mmol, 1 equiv) in MeOH (50 mL) was added HOAc (2.76 mL, 48.28 mmol, 3 equiv). The mixture was stirred at 20° C. for 1 hr. Then NaBH3CN (2.02 g, 32.2 mml, 2 equiv) was added. The mixture was stirred at 20° C. for 1 h under nitrogen atmosphere. The reaction mixture was extracted with water (120 mL) and EtOAc (40 mL×3). The combined organic layers were dried over anhydrous Na2SO., filtered and the filtrate was concentrated under reduced pressure to give a residue. The residue was purified by silica gel column chromatography eluted with CH₂Cl₂/MeOH (10:1) to afford 487-1 (6.64 g, 85.89%) as a yellow solid.

¹H-NMR-487-1: (400 MHz, CHLOROFORM-d) δ 8.80 (s, 1H), 8.10 (d, J=2.0 Hz, 1H), 8.01 (s, 1H), 7.13 (t, J=7.6 Hz, 1H), 6.58 (t, J=7.6 Hz, 2H), 6.52 (s, 1H), 4.49 (s, 2H), 3.80 (d, J=10.4 Hz, 1H), 3.35 (s, 3H), 2.39-2.29 (m, 1H), 1.94-1.76 (m, 6H)

### 2. Synthesis of 487-2

To a solution of 487-1 (6.64 g, 13.8 mmol, 1 equiv) in DCM (66 mL) was added Py. (6.69 mL, 82.94 mmol, 6 equiv) and bis(trichloromethyl) carbonate (2.05 g, 6.91 mmcl, 0.5 equiv) at 0° C. Then the mixture was stirred at 20° C. for 0.5 hr. The reaction was quenched with Sat. NaHCO₃ (300 mL). The aqueous layer was extracted with DCM (3×100 mL). The combined organic layers were dried over anhydrous Na₂SO₄, filtered and the filtrate was concentrated under reduced pressure to give a residue. The residue was purified by silica gel column chromatography elated with CH₂Cl₂/MeOH (10:1) to afford 487-2 (5.34 g, 76.29%) as a yellow solid.

### 3. Synthesis of 487

To a solution of 487-2 (450 mg, 888 umol, 1 equiv) and 487-3 (393 mg, 1.78 mmol, 2 equiv) in THF (8 ml) and H2O (2 mL) was added Xphos Pd G₄ (76.5 mg, 88.8 umol, 0.1 equiv) and Cs2CO₃ (868 mg, 2.67 mmol, 3 equiv). The mixture was stirred at 80° C. for 12 h under nitrogen atmosphere. The reaction mixture was extracted with water (120 mL) and EtOAc (40 mL×3). The combined organic layers were dried over Na2SO₄, filtered and the filtrate was concentrated under reduced pressure to give a residue. The residue was purified by silica gel column chromatography eluted with CH₂Cl₂/MeOH (10:1) to give a crude product. The crude product (500 mg) was purified by prep-HPLC with the following conditions (column: Phenomenex Luna C18 200*40 nm*10um; Mobile Phase A: Water (0.2% formic acid), Mobile Phase B: ACN; Flow rate: 50 ml/min; Gradient: 5% B to 45% B in 8 min; Wave Length: 220 nm; RT1 (min): 7.5) to afford 487 (183.5 mg, 38.01%) as a yellow solid.

MS-487: (ES, m/z): [M+H]⁺ 541.3. ¹H-NMR-487: (400 MHz, METHANOL-d4) δ 8.48 (s, 1H), 8.36 (s, 1H), 7.82 (s, 1H), 7.66-7.61 (m, 2H), 7.48 (t, J=8.0 Hz, 1H), 7.28 (d, J=8.0 Hz, 1H), 7.15 (s, 1H), 7.12 (s, 1H), 4.28 (d, J=10.8 Hz, 1H), 3.54 (s, 3H), 3.23-3.13 (m, 2H), 2.47-2.39 (m, 1H), 2.27-2.21 (m, 1H), 2.14 (t, J=11.2 Hz, 1H), 1.95-1.68 (m, 9H), 1.12-1.00 (m, 1H), 0.95 (d, J=6.4 Hz, 3H).

### Example 479. Synthesis of Compound 488

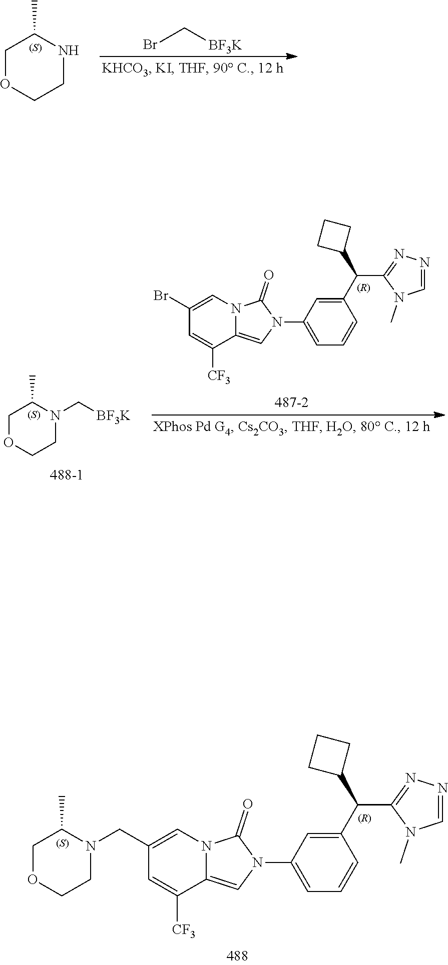

### 1. Synthesis of 488-1

A mixture of (3S)-3-methylmorpholine (2 g, 19.77 mmol, 1 equiv), potassium;bromomethyl (trifluoro)boranuide (3.97 g, 19.7 mmol, 1 equiv), KI (328 mg, 1.98 mmol, 0.1 equiv), KHCO₃ (3.96 g, 39.5 mmol, 2 equiv) in THF (20 mL) was degassed and purged with nitrogen for 3 times, and then the mixture was stirred at 90° C. for 12 h under nitrogen atmosphere. The reaction mixture was concentrated under reduced pressure to give a residue. The residue was triturated with acetone (40 mL) at 40° C. for 1 hr. Then the mixture was filtered and the filtrate was concentrated in vacuum to afford 488-1 (680 mg, crude) as a yellow solid.

### 2. Synthesis of 488

To a solution of 487-2 (260 mg, 513 umol, 1 equiv) and 488-1 (227 mg, 1.03 mmol, 2 equiv) in THF (4 mL) and water (1 mL) was added Cs₂CO₃ (501.94 mg 1.54 mmol, 3 equiv) and dicyclohexyl-[2-(2,4,6-triisopropylphenyl)phenyl]phosphane;methanesulfonate;[2-[2-(methylamino)phenyl] phenyl]palladium(1+) (44.19 mg, 51.35 umol, 0.1 equiv). The mixture was stirred at 80° C. for 12 h under nitrogen atmosphere. The reaction mixture was diluted with water 10 mL and extracted with EtOAc (10 mL×3). The combined organic layers were dried over anhydrous Na2SO4, filtered and concentrated under reduced pressure to give a residue. The residue was purified by flash silica gel chromatography eluted with CH₂Cl₂/MeOH=10:1 to give crude product. The crude product (230 mg) was purified by Prep-HPLC with the following conditions (Column: Phenomenex Luna C18 75*30 mm*3 um; Mobile Phase A: Water (0.2% formic acid), Mobile Phase B: ACN; Flow rate: 50 mL/min; Gradient: 10% B to 40% B in 8 min; Wave Length: 220 nm; RT1 (min): 10.0) to afford 488 (85.4 mg, 31%) as a yellow solid.

MS-488: (ES, m/z): [M+H]⁺ 541.3. ¹H NMR-488 (400 MHz, METHANOL-d₄) δ 8.38 (s, 1H), 7.70 (s, 1H), 7.68-7.63 (m, 2H), 7.50 (t, J=7.6 Hz, 1H), 7.30-7.25 (m, 1H), 7.12 (s, 1H), 7.07 (s, 1H), 4.30 (d, J=10.8 Hz, 1H), 3.90 (d, J=13.6 Hz, 1H), 3.81-3.71 (m, 2H), 3.67-3.59 (m, 1H), 3.56 (s, 3H), 3.30 (s, 1H), 3.07 (d, J=13.6 Hz, 1H), 2.79-2.69 (m, 1H), 2.62-2.51 (m, 1H), 2.34-2.22 (m, 2H), 2.00-1.83 (m, 5H), 1.83-1.73 (m, 1H), 1.12 (d, J=6.4 Hz, 3H).

### Example 480. Synthesis of Compound 489

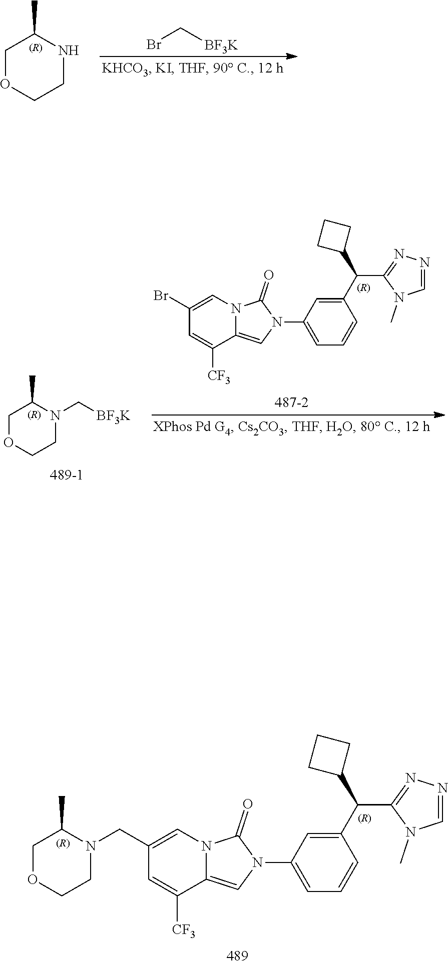

### 1. Synthesis of 489-1

A solution of potassium;bromomethyl(trifluoro)boranuide (3.97 g, 19.7 mmol, 1 equiv), (3R)-3-methylmorpholine (2 g, 19.7 mmol, 1 equiv), KHCO₃ (3.96 g, 39.5 mmol, 2 equiv) and KI (328 mg, 1.98 mmol, 0.1 equiv) in THF (30 mL) was stirred at 90° C. for 12 h under nitrogen atmosphere. The mixture was concentrated under reduced pressure to give a residue. Then the residue was triturated with acetone at 40° C. for 2 h and filtered. The filtrate was concentrated under reduced pressure to afford 489-1 (1.28 g, crude) as a white solid

### 2. Synthesis of 489

To a solution of 489-1 (294 mg, 1.33 mmol, 2 equiv) and 487-2 (337 mg, 665 umol, 1 equiv) in THF (8 mL) and 110 (2 mL) was added Cs₂CO₃ (650 mg, 2.00 mmol, 3 equiv) and dicyclohexyl-[2-(2,4,6-triisopropylphenyl)phenyl]phosphane;methanesulfonate;[2-[2-(methylamino)phenyl] phenyl]palladium(1+) (57.2 mg, 66.5 umol, 0.1 equiv). The mixture was stirred at 80° C. for 12 h under nitrogen atmosphere. The reaction mixture was extracted with water (120 mL) and EtOAc (40 mL×3). The combined organic layers were dried over anhydrous Na₂SO₄, filtered and the filtrate was concentrated under reduced pressure to give a residue. The residue was purified by silica gel column chromatography eluted with CH₂Cl₂/MeOH (10:1) to give a crude product. The crude product (315 mg) was purified by prep-PLC with the following conditions (column: Phenomenex Luna C18 200*40 mm*10un; Mobile Phase A: Water (0.2% formic acid), Mobile Phase B: ACN; Flow rate: 50 mL/min; Gradient: 10% B to 40% B in 8 min; Wave Length: 220 nm; RT1 (min): 8.0) to afford 489 (102.1r mg, 28.03%) as a yellow solid.

MS-489: (ES, m/z): [M+H]⁺ 541.3. ¹H-NMR-489: (400 MHz, METHANOL-d4) δ 8.37 (s, 1H), 8.23 (s, 1H), 7.70 (s, 1H), 7.67-7.61 (m, 2H), 7.48 (t, J=7.6 Hz, 1H), 7.26 (d, J=7.6 Hz, 1H), 7.12 (s, 1H), 7.06 (s, 1H), 4.28 (d, J=11.2 Hz, 1H), 3.90 (d, J=13.6 Hz, 1H), 3.79-3.70 (m, 2H), 3.65-3.57 (m, 1H), 3.54 (s, 3H), 3.31-3.24 (m, 1H), 3.08 (d, J=13.6 Hz, 1H), 2.78-2.71 (m, 1H), 2.62-2.52 (m, 1H), 2.35-2.20 (m, 2H), 1.99-1.81 (m, 5H), 1.81-1.69 (m, 1H), 1.11 (d, J=6.4 Hz, 3H).

### Example 481. Synthesis of Compound 490

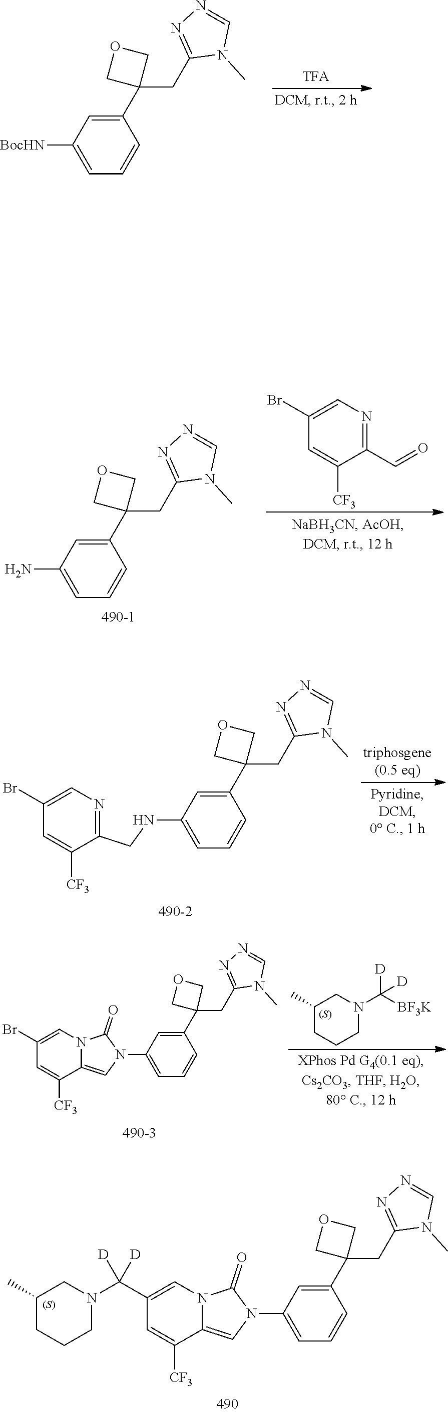

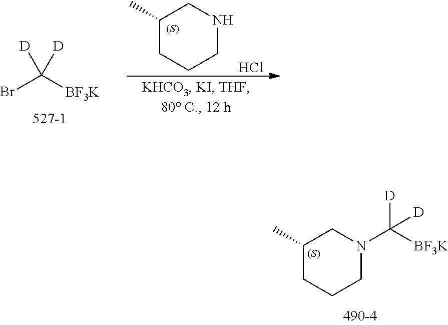

### 1. Synthesis of 490-1

To a solution of tert-butyl N-[3-[3-[(4-methyl-1,2,4-triazol-3-yl)methyl]oxetan-3-yl]phenyl]carbamate (2 g, 5.81 mmol, 1 equiv) in CH2Cl2 (20 mL) was added TFA (5 mL) at 25° C. and stirred for 2 hr. The mixture was concentrated in vacuum to get a crude (4.6 g). The crude (4 g) was dissolved with MeOH (200 mL) and adjust pH=7 with Amberlyst 21 (basic). The mixture was filtered and washed with MeOH (500 mL). The filtrate was concentrated in vacuum to get 490-1 (1.4 g, crude) as a yellow solid.

### 2. Synthesis of 490-2

To a solution of 490-1 (1.3 g, 5.32 mmol, 1.00 equiv) in MeOH (20 mL) was added common int.9 (1.22 g, 4.79 mmol, 0.9 equiv), AcOH (319.57 mg, 5.32 mmol, 1 equiv) at 25° C. and stirred for 1 hr. NaBH3CN (668.83 mg, 10.64 mmol, 2 equiv) was added and stirred for 11 h. The mixture was concentrated in vacuum to get a residue. The residue was diluted with EtOAc (20 mL) and H2O (20 mL), extracted with EtOAc (20 mL×3). The combined organic layers were washed with brine (20 mL×2), dried over Na2SO4, filtered and concentrated under reduced pressure to give a residue. The residue was purified by column chromatography (SiO2) with petroleum ether/EtOAc=1:1 to 0:1 to EtOAc:MeOH=1:0 to 5:1 to get 490-2 (1.3 g, crude) as a yellow solid.

### 3. Synthesis of 490-3

To a solution of 490-2 (400 mg, 829.37 umol, 1 equiv) in DCM (10 ML) was added Py. (393.62 mg, 4.98 mmol, 6 equiv), bis(trichloromethyl) carbonate (0.12 g, 414.68 umol, 0.5 equiv) at 0° C. and stirred for 1 h. The reaction mixture was diluted with Sat.NaHCO₃ (100 mL), extracted with DCM (100 mL×3). The combined organic layers were dried over Na2SO4, filtered and concentrated in vacuum to get a residue. The residue was purified by column chromatography (SiO₂) with petroleum ether/EtOAc=5:1 to 0:1 to DCM/MeOH=1:0 to 1:1 to afford 490-3 (950 mg, crude) as a yellow solid.

### 4. Synthesis of 490-3

To a solution of (3S)-3-methylpiperidine;hydrochloride (668 mg, 4.93 mmol, 1 equiv) in THF (10 mL) was added [bromo(dideuterio)methyl]-trifluoro-boron;potassium hydride (1 g, 4.93 mmol, 1 equiv), KHCO3 (987 mg, 9.86 mmol, 2 equiv), KI (81.8 mg, 492 umol, 0.1 equiv). Then the mixture was heated to 80° C. and stirred for 12 h under N2 atmosphere. The mixture was concentrated in vacuum to get a residue. The residue was dissolved with acetone (150 mL). Then the mixture was heated to 50° C. and stirred for 3 h. The mixture was filtered, the filtrate was concentrated and get 490-4 (1.4 g, crude) was obtained as a light yellow solid.

### 5. Synthesis of 490

To a solution of 490-3 (300 mg, 590.21 umol, 1 equiv) in THF (10 mL), H2O (2.5 mL) was added [dideuterio-[(3S)-3-methyl-1-piperidyl]methyl]-trifluoro-boron;potassium hydride (275.52 mg, 1.25 mmol, 2 equiv), dicyclohexyl-[2-(2,4,6-triisopropylphenyl)phenyl]phosphane;methanesulfonate;[2-[2-(methylamino)phenyl]phenyl]palladium(1+) (53.78 mg, 62.50 umol, 0.1 eq), dicesium;carbonate (610.91 mg, 1.88 mmol, 3 equiv) under N2. The suspension was degassed and purged with N2 for 3 time. The mixture was heated to 80° C. and stirred for 12 h under N2. The mixture was poured into H2O (20 mL), extracted with DCM (20 mL×3). The combined organic layer was dried by Na2SO4, filtered and concentrated in vacuum to get a residue. The residue was purified by column (SiO2) with petroleum ether/EtOAc=5:1 to 0:1 to DCM:MeOH=1:0 to 10:1 to get a crude. The crude was purified by prep-HPLC (column: Phenomenex Luna C18 200*40 mm*10 um; mobile phase: [water (FA)-ACN]; B %: 5%-45%, 8 min, Wave Length: 220 nm; RT (min): 7.8 min) to get 490 (127.5 mg) as a light yellow solid,

MS-490: (ES, m/z): [M+H]+ 543.2. 1H-NMR-490: (400 MHz, MeOD) δ 8.39 (s, 1H), 8.18 (s, 1H), 7.87 (s, 1H), 7.63-7.60 (m, 1H), 7.46 (t, J=8 Hz, 1H), 7.29-7.28 (m, 1H), 7.16-7.12 (m, 2H), 6.93-6.91 (m, 1H), 5.08-5.05 (m, 4H), 3.66 (s, 2H), 3.28-3.21 (m, 2H), 2.96 (s, 3H), 2.58-2.52 (m, 1H), 2.30-2.24 (m, 1H), 1.89-1.82 (m, 3H), 1.79-1.71 (m, 1H), 1.14-1.05 (m, 1H), 0.97 (d, J=6.4 Hz, 3H).

### Example 482. Synthesis of Compound 491

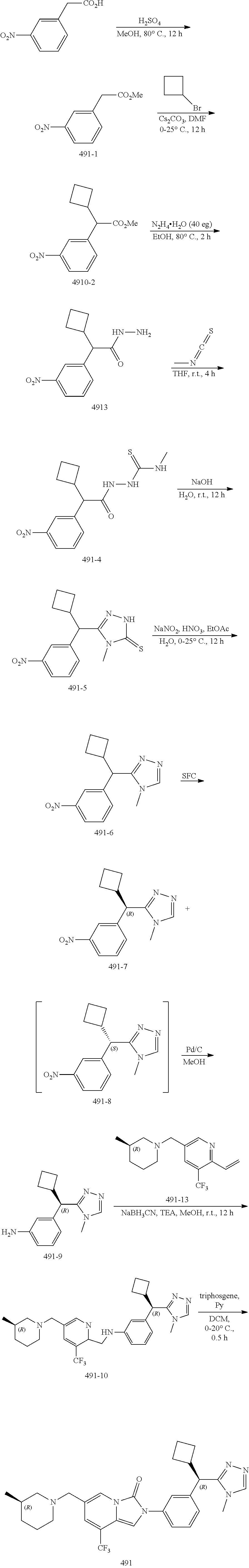

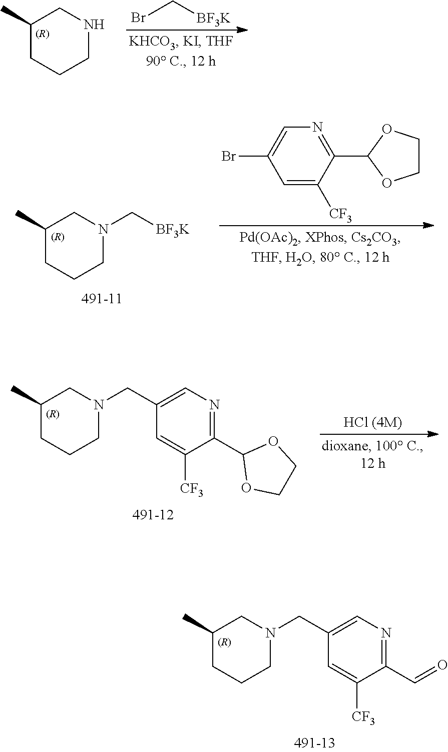

### 1. Synthesis of 491-1

To a solution of 2-(3-nitrophenyl)acetic acid (100.0 g, 552.1 mmol, 1 equiv) in MeOH (1000.0 mL) was added H2SO4 (10.9 g, 110.4 mmol, 5.9 mL, 0.2 equiv). The mixture was stirred at 80° C. for 12 hours. The reaction was poured into water (1000.0 mL) and the resulting mixture was extracted with EtOAc (700.0 mL×2). The organic phase was washed with NaHCO₃ (300.0 mL) and brine (300.0 mL), dried over anhydrous Na2SO4, concentrated in vacuum to give a residue to afford 491-1 (104.0 g, 96.53% yield) was as yellow oil.

H-NMR-491-1: (400 MHz, DMSO-d6) δ ppm 8.17-8.22 (m, 1H), 8.11-8.16 (m, 1H), 7.71-7.78 (m, 1H), 7.59-7.67 (m, 1H), 3.86-3.95 (s, 2H), 3.58-3.68 (s, 3H).

### 2. Synthesis of 491-2

To a solution of 491-1 (30.0 g, 153.7 mmol, 1.0 equiv) in DMF (300.0 mL) was added Cs2CO3 (250.4 g, 768.5 mmol, 5.0 equiv) at 0° C. The mixture was stirred at 0° C. for 3 hours. Then the mixture was added bromocyclobutane (62.3 g, 461.1 mmol, 43.5 mL, 3.0 equiv) at 25° C. The mixture was stirred at 25° C. for 9 hours. The reaction was poured into water (500.0 mL) and the resulting mixture was extracted with EtOAc (300.0 mL×2). The organic phase was washed with brine (300.0 mL), dried over anhydrous Na2SO4, concentrated in vacuum to give a residue. The residue was purified by column chromatography (SiO2, Petroleum ether/Ethyl acetate=0/1 to 3/1) to afford 491-2 (35.0 g, 91.35% yield) as colorless oil.

### 3. Synthesis of 491-3

To a solution of 491-2 (76.0 g, 304.9 mmol, 1.0 equiv) in EtOH (800.0 mL) was added hydrazine;hydrate (622.9 g, 12.2 mol, 604.8 ml, 98% purity, 40.0 equiv). The mixture was stirred at 80° C. for 2 hours. The reaction mixture was concentrated under reduced pressure to remove solvent. The mixture was added H2O (800.0 mL) and EtOAc (800.0 mL). The organic phase was washed with NaHCO₃ (300.0 ml) and brine (300.0 mL), dried over anhydrous Na2SO4, concentrated in vacuum to give a residue. 491-3 (38.0 g, 40.00% yield) was obtained as a yellow solid.

### 4. Synthesis of 491-4

To a solution of 491-3 (35.0 g, 140.4 mmol, 1.0 equiv) in THF (350.0 mL) was added methylimino(thioxo)ethane (20.5 g, 280.8 mmol, 19.2 mL, 2.0 equiv). The mixture was stirred at 25° C. for 4 hours. The reaction was poured to water (600.0 mL) to give white solid. The solid was filtered and concentrated to afford 491-4 (39.0 g, 86.16% yield) as a white solid.

### 5. Synthesis of 491-5

To a solution of NaOH (36.7 g, 918.1 mmol, 8.0 equiv) in H2O (500.0 mL) was added 491-4 (37.0 g, 114.8 mmol, 1.0 equiv). The mixture was stirred at 25° C. for 2 hours. The reaction mixture was acidified by 1M HCl to pH=3-4 to form solid. The solid was filtered and the filter cake was concentrated under reduced pressure to afford 491-5 (39.0 g, crude) as a white solid.

### 6. Synthesis of 491-6

HNO3 (112.6 g, 1.2 mol, 80.5 mL, 68% purity, 10.0 equiv) was added to H2O (599.0 mL) to afford the diluted HNO3 solution (2M, 690.0 mL). To a solution of 491-5 (37.0 g, 121.6 mmol, 1.0 equiv) and NaNO2 (83.8 g, 1.2 mol, 10.0 equiv) in 1120 (420.0 mL) and EtOAc (42.0 mL) was added diluted HNO3 solution (1 M, 6.2 mL) dropwise at 0° C. Then the mixture was stirred at 25° C. for 12 hours. The reaction mixture was neutralized by a.q, NaHCO₃ to pH=7-8, and the resulting mixture was extracted with EtOAc (800.0 mL×2). The organic phase was washed with brine (500.0 mL), dried over anhydrous Na2SO4, concentrated in vacuum to give 491-6 (34.0 g, 98.19% yield) as a white solid.

H-NMR-491-6: (400 MHz, DMSO-d6) 8.36 (s, 1H), 8.14-8.20 (m, 1H), 8.06-8.13 (m, 1H), 7.76 (br d, J=7.6 Hz, 1H), 7.58-7.67 (m, 1H), 4.42-4.54 (m, 1H), 3.45 (s, 3H), 3.05-3.21 (m, 1H), 1.99-2.12 (m, 1H), 1.65-1.88 (m, 5H).

### 7. Synthesis of 491-7&8

The racemate was purified by SFC (column: DAICEL CHIRALPAK IC (250 mm*50 mm, 10 um);mobile phase: [0.1% NH3H2O MEOH]; B %: 55%-55%, 9.5 min) to afford 491-7 (29 g, 48.00% yield) as a yellow solid and 491-8 (29 g, 48.00% yield) as a yellow solid.

H-NMR-491-7: (400 MHz, DMSO-d6) δ ppm 8.32-8.42 (m, 1H), 8.13-8.19 (m, 1H), 8.07-8.13 (m, 1H), 7.72-7.80 (m, 1H), 7.58-7.68 (m, 1H), 4.43-4.52 (m, 1H), 3.39-3.51 (m, 3H), 3.07-3.16 (m, 1H), 1.99-2.11 (m, 1H), 1.67-1.86 (m, 5H).

H-NMR-491-8: (400 MHz, DMSO-d6) δ ppm 8.34-8.42 (m, 1H), 8.14-8.19 (m, 1H), 8.06-8.13 (m, 1H), 7.72-7.79 (m, 1H), 7.58-7.67 (m, 1H), 4.39-4.53 (m, 1H), 3.45 (s, 3H), 3.07-3.17 (m, 1H), 1.98-2.13 (m, 1H), 1.64-1.83 (m, 5H).

### 8. Synthesis of 491-9

A mixture of 491-7 (2.0 g, 7.3 mmol, 1.0 equiv), Pd/C (1.0 g, 10% purity) in MeOH (15.0 mL) was degassed and purged with H2 for 3 times, and then the mixture was stirred at 30° C. for 12 hours under 1-12 atmosphere (50 psi). The reaction mixture was filtered and the filtrate was concentrated to afford 491-9 (1.7 g, 95.52% yield) as a black solid.

H-NMR-491-9: (400 MHz, DMSO-d6) δ ppm 8.22-8.33 (m, 1H), 6.86-6.96 (m, 1H), 6.28-6.43 (m, 3H), 4.90-5.10 (m, 2H), 3.85-3.95 (m, 1H), 3.31-3.35 (m, 3H), 3.01-3.16 (m, 1H), 1.95-2.16 (m, 1H), 1.71-1.80 (m, 4H), 1.58-1.68 (m, 1H).

### 9. Synthesis of 491-10

To a solution of 491-9 (450 mg 1.8 mmol, 1.0 equiv) in MeOH (10.0 mL) was added HOAc (223 mg, 3.7 mmol, 2.0 equiv) dropwise and 5-[[(3R)-3-methyl-1-piperidyl]methyl]-3-(trifluoromethyl)pyridine-2-carbaldehyde (532 mg, 1.8 mmol, 1.0 equiv) at 20° C. After addition, the mixture was stirred at this temperature for 1 hour, and then NaBH3CN (233 mg, 3.7 mmol, 2.0 equiv) was added at 20° C. The resulting mixture was stirred at 20° C. for 11 hours. The reaction was poured into water (20.0 mL) and the resulting mixture was extracted with DCM (10.0 mL×2). The organic phase was washed with brine (10.0 mL), dried over anhydrous Na2SO4, concentrated in vacuum to give a residue. The residue was purified by column chromatography (SiO2, Dichloromethane: Methanol=30/1 to 1/1) to afford (680 mg, 71.32% yield) as a white solid.

### 10. Synthesis of 491

To a solution of 491 (170 mg, 1.0 equiv) in DCM (5.0 mL) was added dropwise pyridine (157 mg, 1.9 mmol, 6.0 equiv) at 0 C. And then triphosgene (49 mg, 0.5 equiv) in DCM (0.5 mL) was added dropwise at 0° C. The resulting mixture was stirred at 20° C. for 0.5 hour. The reaction was poured into water (20.0 mL) and the resulting mixture was extracted with EtOAc (2×15.0 mL). The organic phase was washed with brine (10.0 mL), dried over anhydrous Na2SO4, concentrated in vacuum to give a residue. The residue was purified by prep-HPLC (column: Phenomenex Luna C18 200*40 mm*10 um; mobile phase: [water (FA)-ACN]; B %: 5%-45%, 8 min) to afford 491 (114 mg, 62.23% yield) as a yellow solid.

LCMS-491 (ES, m/z): [M+H]+ 539.3. H-NMR-491 (400 MHz, METHANOL-d4) δ ppm 8.41-8.50 (m, 1H), 8.32-8.39 (m, 1H), 7.77-7.88 (m, 1H), 7.59-7.68 (m, 2H), 7.49 (t, J=7.6 Hz, 1H), 7.24-7.32 (m, 1H), 7.14 (br d, J=14.6 Hz, 2H), 4.23-4.34 (m, 1H), 3.67-3.80 (m, 2H), 3.50-3.59 (m, 3H), 3.25-3.30 (m, 1H), 3.10-3.24 (m, 2H), 2.36-2.48 (m, 1H), 2.19-2.30 (m, 1H), 2.08-2.18 (m, 1H), 1.86-1.99 (m, 4H), 1.64-1.84 (m, 5H), 1.01-1.12 (m, 1H), 0.90-0.98 (m, 3H).

### 11. Synthesis of 491-11

To a solution of (3R)-3-methylpiperidine (1.5 g, 15.1 mmol, 1 equiv) and potassium;bromomethyl (trifluoro)boranuide (3.04 g, 15.1 mmol, 1 equiv) in THF (30 mL) was added KHCO3 (3.03 g, 30.2 mmol, 2 equiv) and KI (251 mg, 1.51 mmol, 0.1 equiv). The mixture was stirred at 90° C. for 12 hi under nitrogen atmosphere. The mixture was concentrated under reduced pressure. The residue was triturated with acetone (50 ml) at 35° C. for 30 min. Then the mixture was filtered, and the filtrate was concentrated under reduced pressure to afford 491-11 (2.5 g, crude) as a yellow oil.

### 12. Synthesis of 491-12

To a solution of 55-bromo-2-(1,3-dioxolan-2-yl)-3-(trifluoromethyl)pyridine (1.68 g, 5.64 mmol, 1 equiv) and 491-11 (2.47 g, 11.2 mmol, 2 equiv) in THF (32 mL) and water (8 mL) was added dicyclohexyl-[2-(2,4,6-triisopropylphenyl)phenyl]phosphane;methanesulfonate;[2-[2-(methylamino)phenyl]phenyl]palladium(1+4) (485 mg, 563 umol, 0.1 equiv), Cs2CO3 (5.51 g, 16.9 mmol, 3 equiv), the mixture was stirred at 80° C. for 12 hr under nitrogen atmosphere. The reaction mixture was diluted with water (40 mL), extracted with EtOAc (35 mL×3). The combined organic layers were dried over anhydrous Na2SO4, filtered and concentrated in vacuum to give a residue. The residue was purified by silica gel column chromatography, eluted with PE/EA=1:1 to afford 491-12 (1.7 g, 91% yield) as a yellow oil.

H-NMR-491-12: (400 MHz, CHLOROFORM-d) δ 8.78 (s, 1H), 7.98 (s, 1H), 6.28 (d, J=1.2 Hz, 1H), 4.41-4.33 (m, 2H), 4.18-4.10 (m, 2H), 3.55 (d, J=2.0 Hz, 2H), 2.81-2.64 (m, 2H), 2.00-1.89 (m, 1H), 1.75-1.56 (m, 5H), 0.93-0.83 (m, 4H)

### 13. Synthesis of 491-13

To a solution of 491-12 (1.7 g, 5.15 mmol, 1 equiv) in dioxane (17 mL) was added HC (4 M, 17 mL, 13.21 equiv), the mixture was stirred at 100° C. for 12 hr. The reaction mixture was diluted with Sat.NaHCO₃ (50 mL), extracted with EtOAc (30 mL×3). The combined organic layers were dried over anhydrous Na2SO4, filtered and concentrated in vacuum. The residue was purified by silica gel column chromatography, eluted with PE/EtOAc=6:1 to afford 491-13 (1 g, 68%) as a yellow oil.

H-NMR-491-13: (400 MHz, CHLOROFORM-d) δ 10.21 (s, 1H), 8.87 (s, 1H), 8.12 (s, 1H), 3.59 (s, 2H), 2.76-2.64 (m, 2H), 2.02-1.90 (m, 1H), 1.75-1.60 (m, 5H), 1.59-1.51 (m, 1H), 0.94-0.86 (m, 1H), 0.84 (d, J=6.0 Hz, 3H).

### Example 483. Synthesis of Compound 492

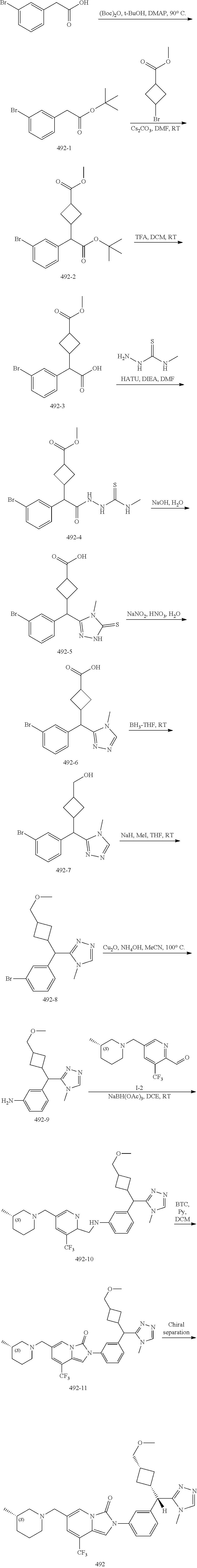

### 1. Synthesis of 492-1

To a stirred solution of M-bromophenylacetic acid (20 g 93,003 mmol, 1 equiv) and DMAP (1.14 g, 9.300 mmol, 0.1 equiv) in t-BuOH (300 mL) was added (Boc)20 (49.60 g, 186.006 mmol, 2 equiv) at room temperature under nitrogen atmosphere. The resulting mixture was stirred for 6 h at 90° C. under nitrogen atmosphere. The reaction was quenched with water at room temperature. The aqueous layer was extracted with EtOAc (2×200 mL). The resulting mixture was concentrated under vacuum. The residue was purified by silica gel column chromatography, eluted with PE/EA (50:1) to afford tert-butyl 2-(3-bromophenyl)acetate (16 g, 57.10%) as a light yellow oil.

### 2. Synthesis of 492-2

To a stirred solution of 492-1 (8 g, 29.504 mmol, 1 equiv) in DMF (100 mL) was added Cs₂CO₃ (48.06 g, 147.520 mmol, 5 equiv) at 0° C. under nitrogen atmosphere. To the above mixture was added methyl 3-bromocyclobutane-1-carboxylate (17.09 g, 88.512 mmol, 3 equiv) at 0° C. The resulting mixture was stirred for additional overnight at room temperature. The reaction was quenched with NH4Cl (aq.) at room temperature. The aqueous layer was extracted with EtOAc (2×200 mL). The resulting mixture was concentrated under vacuum. The residue was purified by silica gel column chromatography, eluted with PE/EA (50:1) to afford 492-2 (7.4 g, 58.90%) as a light yellow oil.

### 3. Synthesis of 492-3

To a stirred solution of 492-2 (7.4 g, 19.307 mmol, 1 equiv) in DCM (50 mL) was added TFA (10 mL) at room temperature. The resulting mixture was stirred for overnight at room temperature. The resulting mixture was concentrated under vacuum. The crude product was used in the next step directly without further purification. This resulted in 492-3 (7.4 g, 97.23%) as a brown oil.

### 4. Synthesis of 492-4

To a stirred solution of 492-3 (7.3 g, 22.312 mmol, 1 equiv) and HATU (12.73 g, 33.468 mmol, 1.5 equiv) in DMF (100 mL) were added DIEA (8.65 g, 66.936 mmol, 3 equiv) and 1-amino-3-methylthiourea (3.05 g, 29.006 mmol, 1.3 equiv) at room temperature. The resulting mixture was stirred for 3 h at room temperature. The resulting mixture was diluted with water (300 mL). The aqueous layer was extracted with EtOAc (2×200 mL). The resulting mixture was concentrated under vacuum. The residue was purified by silica gel column chromatography, eluted with PE/EA (1:1) to afford 492-4 (6 g, 51.92%) as a brown yellow oil.

### 5. Synthesis of 492-5

To a stirred solution of NaOH (4.63 g, 115,856 mmol, 8 equiv) in H₂O (100 mL) was added 492-4 (6 g, 14.482 mmol, 1 equiv) at room temperature. The resulting mixture was stirred for 6 h at room temperature. The mixture was neutralized to pH 7 with HCl (aq). The resulting mixture was concentrated under vacuum. The crude product was used in the next step directly without further purification. This resulted in 492-5 (5 g, 72.25%) as a yellow solid.

### 6. Synthesis of 492-6

To a stirred solution of 492-5 (5 g, 13.079 mmol, 1 equiv) and NaNO₂ (9.02 g, 130,790 mmol, 10 equiv) in 0H₂O (200 mL) was added HNO₃ (130.7 mL g, 130.790 mmol, 10.00 equiv, 1 M) at room temperature. The resulting mixture was stirred for 4 h at room temperature. The mixture was a neutralized to pH 7 with saturated NaHCO₃ (aq.). The resulting mixture was concentrated under vacuum. The residue was dissolved in DCM/MeOH=10:1 (300 mL). The resulting mixture was filtered, the filter cake was washed with DCM (2×50 mL). The filtrate was concentrated under reduced pressure. The crude product was used in the next step directly without further purification. This resulted in 492-6 (4 g, 69.86%) as a yellow solid.

### 7. Synthesis of 492-7

To a stirred solution of 492-6 (3 g, 9.484 mmol, 1 equiv) in THF) was added BH₃-THF (42.83 mL, 42.830 mmol, 5 equiv) dropwise at 0° C. under nitrogen atmosphere. The resulting mixture was stirred for overnight at room temperature. The reaction was quenched with NH4Cl (aq.) at room temperature. The aqueous layer was extracted with EtOAc (3×100 mL). The combined organic layers were concentrated under reduced pressure. The residue was purified by reverse flash chromatography with the following conditions: column, C18 silica gel; mobile phase, MeCN in Water (10 mmol/L NH₄HCO₃), 10% to 50% gradient in 20 min; detector, UV 220 nm. This resulted in 492-7 (1.3 g, 43.52%) as a off-white solid.

### 8. Synthesis of 492-8

To a stirred solution of 492-7 (1.27 g, 3.777 mmol, 1 equiv) in THF (20 ML) was added NaH (0.18 g, 4.532 mmol, 1.2 equiv, 60%) at 0° C. under nitrogen atmosphere. The resulting mixture was stirred for 0.5 h at 0° C. under nitrogen atmosphere. To the above mixture was added MeI (0.80 g, 5.665 mmol, 1.5 equiv). The resulting mixture was stirred for additional overnight at room temperature. The reaction was quenched by the addition of sat. NH4Cl (aq.) (60 mL). The resulting mixture was extracted with EtOAc (3×60 mL). The combined organic layers were concentrated under reduced pressure. The residue was purified by silica gel column chromatography, elated with CH₂Cl₂/MeOH (50:1) to afford 492-8 (920 mg, 69.54%) as a white solid.

### 9. Synthesis of 492-9

To a solution of 492-8 (920 mg, 2.627 mmol, 1 equiv) in MeCN (18 mL) and NH₄OH (6 mL) was added Cu2O (150.34 mg, 1,051 mmol, 0.4 equiv) in a pressure tank. The resulting mixture was stirred for overnight at 100° C. The resulting mixture was diluted with water (60 mL). The resulting mixture was extracted with CH₂Cl₂/MeOH=10/1 (3×50 mL). The combined organic layers were concentrated under reduced pressure. The residue was purified by silica gel column chromatography, eluted with CH₂Cl₂/MeOH (25:1) to afford 492-9 (750 mg, 99.713%) as a white solid.

### 10. Synthesis of 492-10

A solution of 492-9 (750 mg, 2,619 mmol, 1 equiv) and I-2 (899.75 mg, 3.143 mmol, 1.2 equiv) in DCE (15 mL) was stirred for overnight at room temperature. To the above mixture was added NaBH(OAc)₃ (1110.10 mg, 5.238 mmol, 2 equiv) at room temperature. The resulting mixture was stirred for additional 6 h at room temperature. The resulting mixture was diluted with water (30 mL). The resulting mixture was extracted with CH₂Cl₂/MeOH=1-10/1 (3×30 mL). The combined organic layers were concentrated under reduced pressure. The residue was purified by silica gel column chromatography, eluted with CH2Cl₂/MeOH (30:1) to afford 492-10 (1 g, 68.59%) as a white solid.

### 11. Synthesis of 492-11

To a stirred solution of 492-10 (0.98 g, 1.760 mmol, 1 equiv) and Pyridine (0.84 g, 10.560 mmol, 6 equiv) in DCM (20 mL) was added Triphosgene (0.21 g, 0.704 mmol, 0.4 equiv). The resulting mixture was stirred for 2 h at room temperature under nitrogen atmosphere. The reaction was quenched by the addition of sat. NaHCO₃ (aq.) (40 mL) at room temperature. The resulting mixture was extracted with CH₂Cl₂/MeOH=10/1 (3×40 ml). The combined organic layers were concentrated under reduced pressure. The residue was purified by reverse flash chromatography with the following conditions: column, C18 silica gel; mobile phase, MeCN in Water (10 mmol/L NH₄HCO₃), 5% to 70% gradient in 30 min; detector, UV 254 nm. This resulted in 492-11 (700 mg, 68.24%) as a yellow solid.

### 12. Synthesis of 492

492-11 (700 mg) was purified by Prep-Chiral-SFC with the following conditions (Column: UniChiral OD-5H, 3*25 cm, 5 μm; Mobile Phase A: CO₂, Mobile Phase B: MEOH (0.31% 2M N13); Flow rate: 70 mL/min; Gradient: isocratic 35% B; Column Temperature (° C.): 35; Back Pressure (bar): 100; Wave Length: 220 nm; RT1 (min): 4.56; RT2 (min): 5.92; first peak was product) to afford crude product (350 mg). The crude product (350 mg) was purified by Prep-HPLC with the following conditions (Column: CHIRALPAK IH, 2*25 cm, 5 μm; Mobile Phase A: Hex (0.5% 2M NH₃-MeOH), Mobile Phase B: EtOH: DCM=1:1; Flow rate: 20 mL/min; Gradient: 20% B to 20% B in 9 min; Wave Length: 220/254 nm; RT1 (min): 4.78; RT2 (min): 7.00; first peak was product) to afford 492 (151.2 mg, 21.60%) as a yellow solid.

LCMS-492 (ES, m/z): [M+H]⁺ 583. NMR-492 (400 MHz, CD₃OD, δ ppm): 0.92-0.99 (m, 4H), 1.53-1.79 (m, 7H), 1.96-1.99 (m, 1H), 2.01-2.13 (m, 1H), 2.33-2.36 (m, 1H), 2.45-2.47 (m, 1H), 2.94-3.02 (m, 2H), 3.15-3.18 (m, 1H), 3.37 (s, 5H), 3.43 (s, 2H), 3.54 (s, 3H), 4.22-4.25 (d, 1H), 7.13-7.14 (d, 2H), 7.26-7.28 (d, 1H), 7.51-7.53 (t, 1H), 7.65-7.71 (m, 3H), 8.38 (s, 1H).

### Example 484. Synthesis of Compound 493

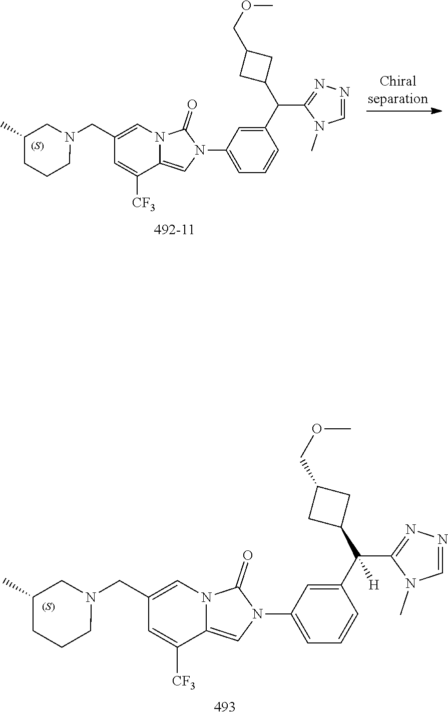

### 1. Synthesis of 493

492-11 (700 mg, 1,201 mmol, 1 equiv) was purified by Prep-Chiral-SFC with the following conditions (Column: UniChiral OD-5H, 3*25 cm, 5 μm; Mobile Phase A: CO₂, Mobile Phase B: MEOH (0.1% 2M NH₃); Flow rate: 70 mL/min; Gradient: isocratic 35% B; Column Temperature (° C.): 35; Back Pressure (bar): 100; Wave Length: 220 nm; RT1 (min): 4.56; RT2 (min): 5.92; the first peak was product) to afford crude product (350 mg). The crude product (350 mg) was purified by Prep-Chiral-HPLC with the following conditions (Column: CHIRALPAK IH, 2*25 cm, 5 um; Mobile Phase A: Hex (0.5% 2M NH₃-MeOH), Mobile Phase B: EtOH:DCM=1:1; Flow rate: 20 m/min; Gradient: 20% B to 20% B in 9 min; Wave Length: 220/254 nm; RT1 (main): 4.78; RT2 (min): 7.00; the second peak was product) to afford 493 (151.2 mg, 21.60%) as a yellow solid.

LCMS-493 (ES, m/z): [M+H]⁺ 583. NMR-493 (400 MHz, CD₃OD, δ ppm): 0.91-0.98 (m, 4H), 1.53-1.89 (m, 7H), 1.99-2.12 (m, 3H), 2.56-2.57 (m, 1H), 2.89-3.95 (m, 2H), 3.35-3.47 (m, 6H), 3.48-3.49 (d, 2H), 3.56 (s, 3H), 4.35-4.37 (d, 1H), 7.13 (s, 2H), 7.28-7.30 (d, 1H), 7.51-7.53 (t, 1H), 7.65-7.70 (m, 3H), 8.38 (s, 1H).

### Example 485. Synthesis of Compound 494

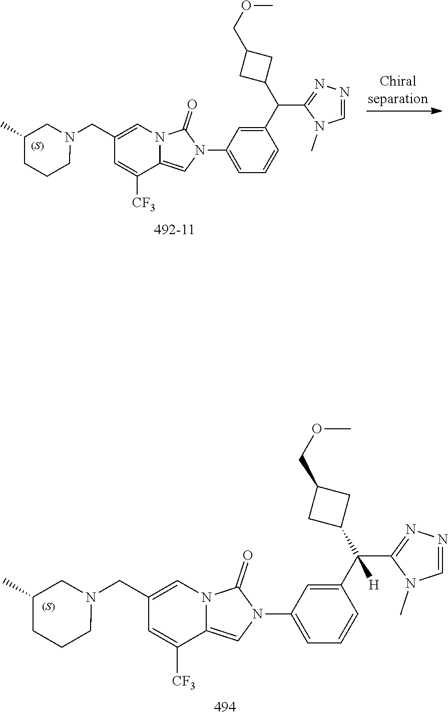

### 1. Synthesis of 494

492-11 (700 mg) was purified by Prep-Chiral-SFC with the following conditions (Column: UniChiral OD-SH, 3*25 cm, 5 μm; Mobile Phase A: CO2, Mobile Phase B: MECH (0.1% 2M NH₃); Flow rate: 70 mL/min; Gradient: isocratic 35% B; Column Temperature (° C.): 35; Back Pressure (bar): 100; Wave Length: 220 nm; RT1 (min): 4.56; RT2 (min): 5.92; the second peak was product) to afford crude product (270 mg). The crude product (270 mg) was purified by Prep-HPLC with the following conditions (Column: CHIRAL ART Cellulose-SB, 2*25 cm, 5 n; Mobile Phase A: Hex (0.5% 2M NH₃-MeOH), Mobile Phase B: EtOH:DCM=1:1; Flow rate: 20 mL/min; Gradient: 25% B to 25% B in 9.5 min; Wave Length: 220/254 am; RT1 (min): 5.73; RT2 (min): 6.76; the second peak was product) to afford 494 (104.7 mg, 14.96%) as a yellow solid.

LCMS-494 (ES, m/z): [M+H]⁺ 583. NMR-494 (400 MHz, CD₃OD, δ ppm): 0.90-0.95 (m, 4H), 1.53-1.78 (m, 6H), 1.84-1.87 (m, 1H), 2.01-2.10 (m, 3H), 2.55-2.56 (m, 1H), 2.89-2.96 (m, 2H), 3.30-3.32 (m, 1H), 3.35-3.39 (m, 5H), 3.47-3.49 (d, 2H), 3.56 (s, 3H), 4.35-4.37 (d, 1H), 7.13-7.14 (d, 2H), 7.28-7.30 (d, 1H), 7.51-7.53 (t, 1H), 7.65-7.70 (m, 3H), 8.38 (s, 1H).

### Example 486. Synthesis of Compound 495

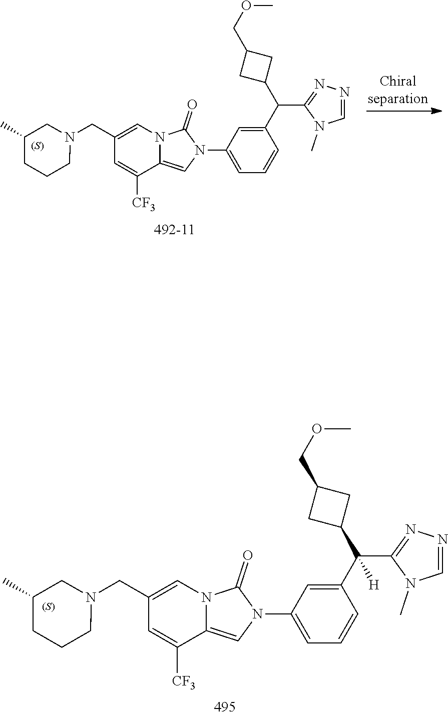

### 1. Synthesis of 495

492-11 (700 mg) was purified by Prep-Chiral-SFC with the following conditions (Column: UniChiral OD-5H, 3*25 cm, 5 μm; Mobile Phase A: CO₂ Mobile Phase B: MEOH (0.1% 2M NH₃); Flow rate: 70 mL/min; Gradient: isocratic 35% B; Column Temperature (° C.): 35; Back Pressure (bar): 100; Wave Length: 220 ran; RT1 (min): 4.56; RT2 (min): 5.92; the second peak was product) to afford crude product (350 mg). The crude product (350 mg) was purified by Prep-HPLC with the following conditions (Column: CHIRAL ART Cellulose-SB, 2*25 cm, 5 n; Mobile Phase A: Hex (0.5% 2M NH₃-MeOH), Mobile Phase B: EtOH:DCM=1:1; Flow rate: 20 mL/min; Gradient: 25% B to 25% B in 9.5 min; Wave Length: 220/254 nm; RT1 (min): 5.73; RT2 (min): 6.76; the first peak was product) to afford 495 (104.7 mg, 14.96%) as a yellow solid.

LCMS-495 (ES, m/z): [M+H]⁺ 583. NMR-495 (400 MHz, CD₃OD, δ ppm): 0.91-0.96 (m, 4H), 1.53-1.55 (m, 1H), 1.66-1.79 (m, 6H), 1.96-2.13 (m, 2H), 2.33-2.35 (m, 1H), 2.45-2.47 (m, 1H), 2.93-3.02 (m, 2H), 3.15-3.18 (m, 1H), 3.37-3.47 (m, 5H), 3.50-3.55 (m, 5H), 4.22-4.25 (d, 1H), 7.13-7.14 (d, 2H), 7.26-7.28 (d, 1H), 7.51-7.53 (t, 1H), 7.65-7.70 (m, 3H), 8.38 (s, 1H).

### Example 487. Synthesis of Compound 496

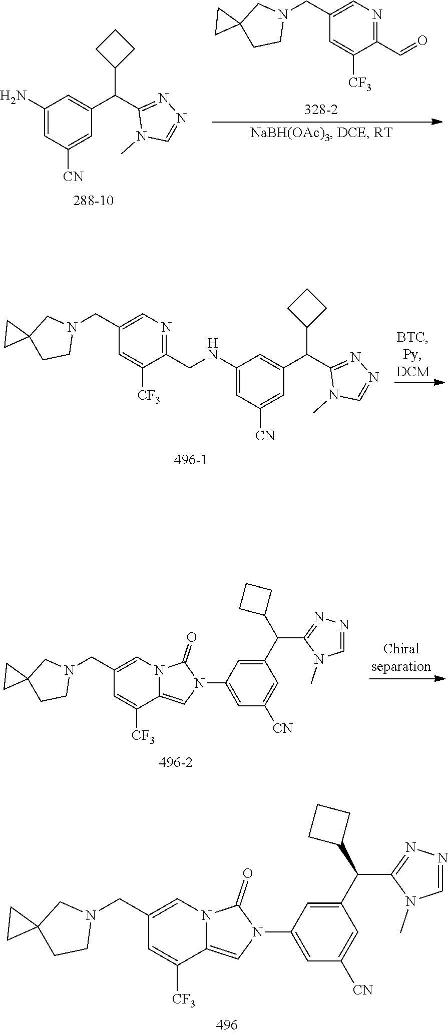 [SMILES: Cn1cnnc1C(c1cc(N)cc(C#N)c1)C1CCC1 ; O=Cc1ncc(CN2CCC3(CC3)C2)cc1C(F)(F)F ; "NaBH(OAc)3, DCE, RT" ; Cn1cnnc1C(c1cc(C#N)cc(NCc2ncc(CN3CCC4(CC4)C3)cc2C(F)(F)F)c1)C1CCC1 ; Cn1cnnc1C(c1cc(C#N)cc(-n2cc3c(C(F)(F)F)cc(CN4CCC5(CC5)C4)cn3c2=O)c1)C1CCC1 ; Cn1cnnc1[C@@H](c1cc(C#N)cc(-n2cc3c(C(F)(F)F)cc(CN4CCC5(CC5)C4)cn3c2=O)c1)C1CCC1]

### 1. Synthesis of 496-1

To a stirred solution of 288-10 (500 mg, 1.870 mmol, 1 equiv) in DCE (10 mL) was added 328-2 (691.20 mg, 2,431 mmol, 1.3 equiv) at room temperature. The resulting mixture was stirred for 3 h at room temperature. To the above mixture was added NaBH(OAc)3 (1189.18 mg, 5.610 mmol, 3 equiv) at room temperature. The resulting mixture was stirred for additional overnight at room temperature. The reaction was quenched with saturated NaHCO₃ (aq.) (50 mL) at room temperature. The aqueous layer was extracted with DCM (3×30 mL). The resulting mixture was concentrated under vacuum. The residue was purified by Prep-TLC (DCM/MeOH 20:1) to afford 496-1 (470 mg, 44.57%) as an off-white solid.

### 2. Synthesis of 496-2

To a stirred solution of 496-1 (450 mg, 0.840 mmol, 1 equiv) and Pyridine (664.56 mg, 8,400 mmol, 10 equiv) in DCM (10 mL) was added Triphosgene (99.72 mg, 0.336 mmol, 0.4 equiv) at room temperature. The resulting mixture was stirred for 10 min at room temperature. The reaction was quenched with saturated NaHCO3 (aq.) (80 mL) at room temperature. The aqueous layer was extracted with DCM (3×30 mL). The resulting mixture was concentrated under vacuum. The residue was purified by Prep-TLC (DCM/MeOH 15:1) to afford 496-2 (300 mg, 61.67%) as a yellow solid.

### 3. Synthesis of 496

The 496-2 (300 mg) was purified by Prep-Chiral-HPLC with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 11 min; Wave Length: 220/254 nm; RT1 (min): 5.50; RT2 (min): 8.07; the second peak is product) to afford 496 (139.7 mg, 46.10%) as a yellow solid.

LC-MS-496 (ES, m/z): [M+H]⁺ 562. H-NMR-496 (400 MHz, DMSO-d6, δ ppm): 0.51-0.58 (m, 4H), 1.75-1.84 (m, 7H), 2.03-2.09 (m, 1H), 2.47-2.50 (m, 2H), 2.64-2.74 (m, 2H), 3.19-3.23 (m, 1H), 3.25 (s, 2H), 3.48 (s, 3H), 4.40-4.42 (d, 1H), 7.07 (s, 1H), 7.51 (s, 1H), 7.68-7.69 (m, 2H), 8.22-8.25 (d, 2H), 8.36 (s, 1H).

### Example 488. Synthesis of Compound 497

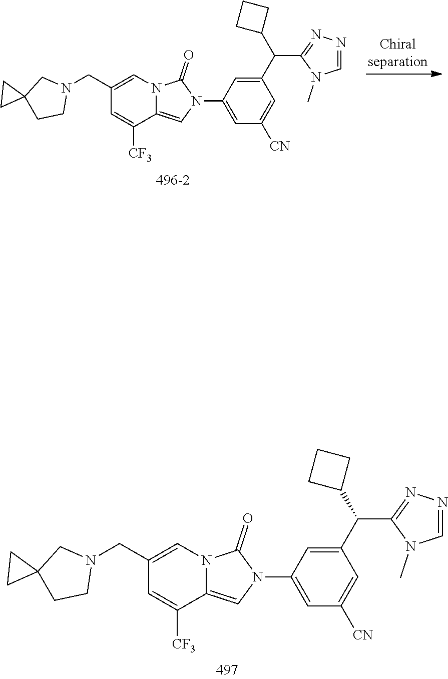 [SMILES: Cn1cnnc1C(c1cc(C#N)cc(-n2cc3c(C(F)(F)F)cc(CN4CCC5(CC5)C4)cn3c2=O)c1)C1CCC1 ; Cn1cnnc1[C@H](c1cc(C#N)cc(-n2cc3c(C(F)(F)F)cc(CN4CCC5(CC5)C4)cn3c2=O)c1)C1CCC1]

### 1. Synthesis of 497

The 496-2 (300 mg) was purified by Prep-Chiral-HPLC with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/in; Gradient: 40% B to 40% B in 11 min; Wave Length: 220/254 am; RT1 (min): 5.50; RT2 (min): 8.07; the first peak is product) to afford 497 (135.1 mg, 44.63%) a a yellow solid.

LC-MS-497 (ES, m/z): [M+H]⁺ 562. H-NMR-497 (400 MHz, DMSO-d6, δ ppm): 0.51-0.58 (m, 4H), 1.75-1.84 (m, 4H), 2.03-2.09 (m, 1H), 2.47-2.50 (m, 2H), 2.64-2.74 (m, 2H), 3.19-3.23 (m, 1H), 3.25 (s, 2H), 3.48 (s, 3H), 4.40-4.42 (d, 1H), 7.07 (s, 1H), 7.51 (s, 1H), 7.69 (s, 1H), 8.22-8.25 (d, 2H), 8.36 (s, 1H).

### Example 489. Synthesis of Compound 498

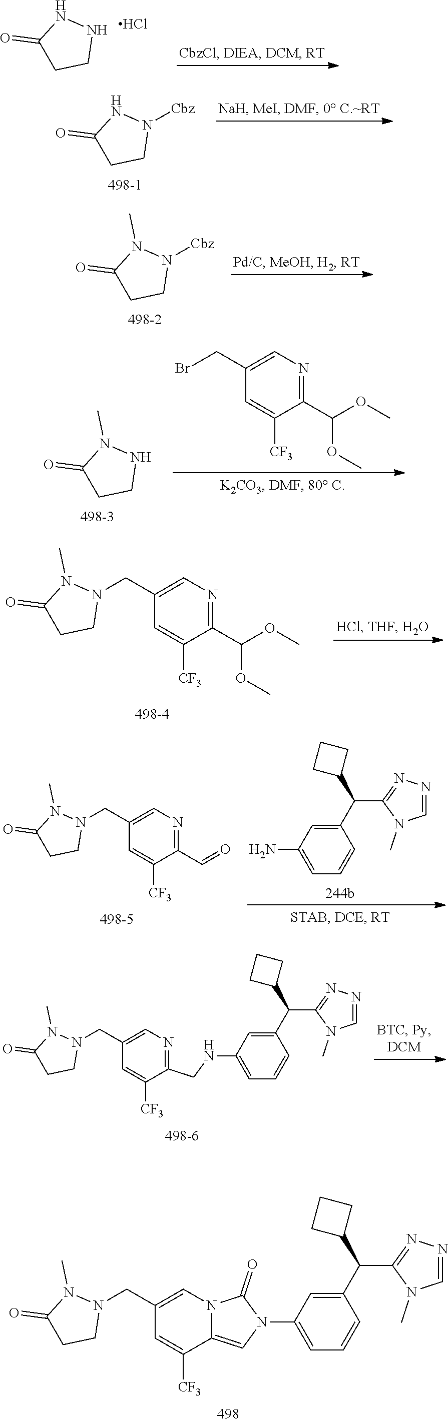

### 1. Synthesis of 498-1

To a stirred solution of pyrazolidin-3-one hydrochloride (2 g, 16,320 mmol, 1 equiv) and DIEA (4.22 g, 32.640 mmol, 2 equiv) in DCM (20 mL) was added CbzCl (4.18 g, 24.480 mmol, 1.5 equiv) at room temperature. The resulting mixture was stirred overnight at room temperature. The reaction was quenched by the addition of water (100 mL) at room temperature. The aqueous layer was extracted with CH₂Cl₂ (2×20 mL). The resulting mixture was concentrated under reduced pressure. The residue was purified by Prep-TLC (PE/EA=1:1) to afford 498-1 (1.2 g, 33.39%) as a yellow oil.

### 2. Synthesis of 498-2

To a stirred mixture of 498-1 (1.1 g, 4.995 mmol, 1 equiv) in DMF (11 mL) were added NaH (0.40 g, 9.990 mmol, 2 equiv, 60%) at 0° C. The resulting mixture was stirred for 1 h at 0° C. To the above mixture was added MeI (1.42 g, 9.990 mmol, 2 equiv) at 0° C. The resulting mixture was stirred for additional 1 h at room temperature. The reaction was quenched with saturated NH₄Cl (aq.) (50 mL) at room temperature. The aqueous layer was extracted with EtOAc (2×20 mL). The resulting mixture was concentrated under reduced pressure. The residue was purified by Prep-TLC (PE/EA=5:1) to afford 498-2 (800 mg, 68.37%) as a yellow oil.

### 3. Synthesis of 498-3

To a solution of 498-2 (800 mg, 3.415 mmol, 1.00 equiv) in 20 mL MeOH was added Pd/C (10%, 80 mg) under nitrogen atmosphere in a 100 mL round-bottom flask. The mixture was hydrogenated at room temperature for 1 h under hydrogen atmosphere using a hydrogen balloon, filtered through a Celite pad and concentrated under reduced pressure. The resulting mixture was filtered, the filter cake was washed with MeOH (3×20 mL). The filtrate was concentrated under reduced pressure to afford 498-3 (250 mg, 73.12%) as a yellow oil.

### 4. Synthesis of 498-4

To a stirred mixture of 498-3 (250 mg, 2.497 mmol, 1 equiv) and 541-2 (1176.46 mg, 3,745 mmol, 1.5 equiv) in DMF (8 mL) were added K₂CO₃ (690.19 mg, 4.994 mmol, 2 equiv) at room temperature. The resulting mixture was stirred overnight at 80° C. The reaction was quenched with saturated NH₄Cl (aq.) (30 mL) at room temperature. The aqueous layer was extracted with EtOAc (2×20 mL). The resulting mixture was concentrated under reduced pressure. The residue was purified by Prep-TLC (CH₂Cl₂/MeOH=30:1) to afford 498-4 (450 mg, 54.07%) as a yellow solid.

### 5. Synthesis of 498-5

To a stirred solution of 498-4 (430 mg, 1.290 mmol, 1.00 equiv) in THF (1.5 mL) were added HCl (1.5 mL, 1M) at room temperature. The resulting mixture was stirred for 2 h at room temperature. The reaction was quenched with saturated NaHCO₃ (aq.) (20 mL) at room temperature. The resulting mixture was concentrated under reduced pressure. The residue was purified by Prep-TLC (PE/EA=1:1) to afford 498-5 (300 mg, 80.96%) as a yellow oil,

### 6. Synthesis of 498-6

To a stirred mixture of 498-5 (280 mg, 0.975 mmol, 1 equiv) and 244b (283.46 mg, 1.170 mmol, 1.2 equiv) in DCE (5 mL) were added STAB (619:79 mg, 2.925 mmol, 3 equiv) at room temperature. The reaction was quenched with saturated NaHCO₃ (aq.) (50 mL) at room temperature. The aqueous layer was extracted with CH₂Cl₂ (2×20 mL). The resulting mixture was concentrated under reduced pressure. The residue was purified by Prep-TLC (CH₂Cl₂/MeOH=15:1) to afford 498-6 (400 mg, 79.90%) as a yellow solid.

### 7. Synthesis of 498

To a stirred solution of 498-6 (380 mg, 0.740 mmol, 1 equiv) and pyridine (351.17 mg, 4.440 mmol, 6 equiv) in DCM (10 mL) was added Triphosgene (76.84 mg, 0.259 mmol, 0.35 equiv) at 0° C. The resulting mixture was stirred for 10 min at room temperature. The reaction was quenched with saturated NaHCO₃ (aq.) (30 mL) at room temperature. The aqueous layer was extracted with CH₂Cl₂ (2×20 mL). The resulting mixture was concentrated under reduced pressure. The residue was purified by Prep-TLC (CH₂Cl₂/MeOH=15:1) to afford 498 (236.3 mg 58.48%) as a yellow solid.

LC-MS-498 (ES, m/z): [M+H]⁺ 540. H-NMR-498 (400 MHz, CD₃OD-d4, δ ppm): 0.82-0.93 (m, 4H), 1.43-1.53 (m, 1H), 1.53-1.71 (m, 5H), 1.71-1.80 (m, 4H), 1.80-1.95 (m, 4H), 1.95-2.02 (m, 1H), 2.71-2.85 (m, 2H), 3.09-3.12 (m, 1H), 3.64-3.68 (m, 2H), 3.92-3.95 (d, 1H), 4.01-4.03 (m, 2H), 7.00 (s, 1H), 7.21-7.26 (m, 2H), 7.32 (s, 1H), 7.35-7.39 (m, 1H), 7.58-7.60 (d, 1H), 7.66 (s, 1H), 7.73 (s, 1H).

### Example 490. Synthesis of Compound 499

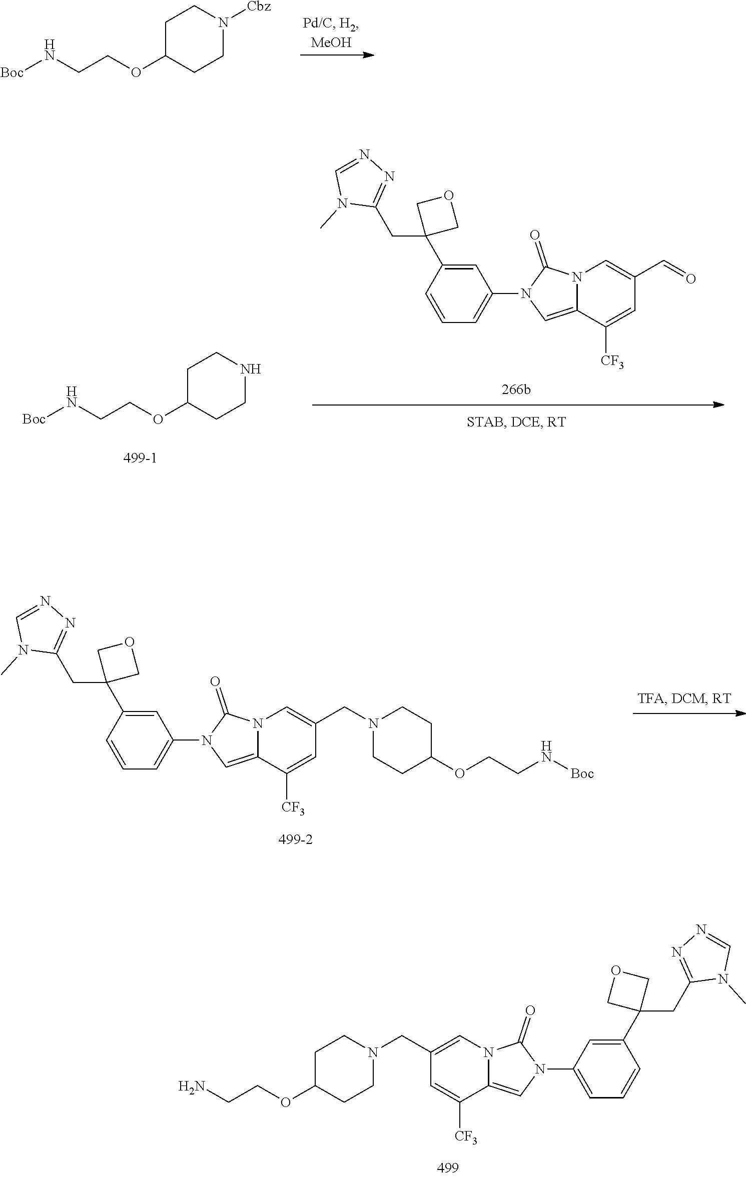 [SMILES: CC(C)(C)OC(=O)NCCOC1CCN(C(=O)OCc2ccccc2)CC1 ; CC(C)(C)OC(=O)NCCOC1CCNCC1 ; Cn1cnnc1CC1(c2cccc(-n3cc4c(C(F)(F)F)cc(C=O)cn4c3=O)c2)COC1 ; Cn1cnnc1CC1(c2cccc(-n3cc4c(C(F)(F)F)cc(CN5CCC(OCCNC(=O)OC(C)(C)C)CC5)cn4c3=O)c2)COC1 ; Cn1cnnc1CC1(c2cccc(-n3cc4c(C(F)(F)F)cc(CN5CCC(OCCN)CC5)cn4c3=O)c2)COC1]

### 1. Synthesis of 499-1

To a solution of benzyl 4-(2-((tert-butoxycarbonyl)amino)ethoxy)piperidine-1-carboxylate (12 g, 31.70 mmol, 1.0 equiv) in 360 mL MeOH was added Pd/C (10%, 2 g) under nitrogen atmosphere in a 500 mL round-bottom flask. The mixture was hydrogenated at room temperature for overnight under hydrogen atmosphere using a hydrogen balloon, filtered through a Celite pad and concentrated under reduced pressure. This resulted in 499-1 (7 g, 90%) as a colorless oil.

### 2. Synthesis of 499-2

To a stirred solution of 499-1 (1 g, 4.09 mmol, 1.0 equiv) and 266b (1.8 g, 4.09 mmol, 1.0 equiv) in DCE (10 mL) was added STAB (1.7 g 8.18 mmol, 2.0 equiv) at room temperature. The resulting mixture was stirred for overnight at room temperature. The reaction was quenched by the addition of water (30 mL) at room temperature. The resulting mixture was extracted with CH2Cl2 (3×30 mL). The combined organic layers were washed with water (3×30 mL), dried over anhydrous Na₂SO₄. After filtration, the filtrate was concentrated under reduced pressure. The residue was purified by Prep-TLC (CH2Cl2/MeOH==10:1) to afford 499-2 (900 mg, 32%) as a yellow solid,

### Example 491. Synthesis of Compound 500

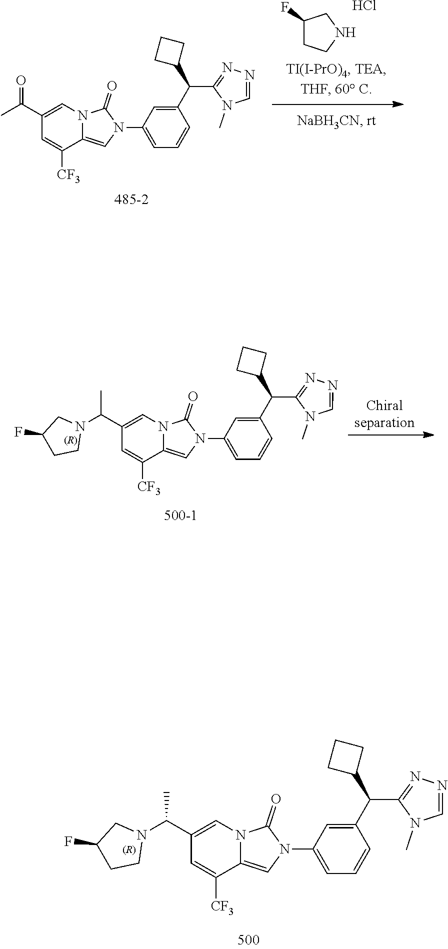

### 1. Synthesis of 500-1

Into a 40 mL vial were added 485-2 (500 mg 1.065 mmol, 1 equiv), (3S)-3-fluoropyrrolidine hydrochloride (113.89 mg, 1.278 mmol, 1.2 equiv), TEA (431.10 mg, 4.260 mmol, 4 equiv), Ti(Oi-Pr)4 (605.41 mg, 2.130 mmol, 2 equiv) and THF (10 mL) at room temperature. The mixture was stirred for 1 h at room temperature. To the above mixture was added NaBH3CN (133.85 mg 2.130 mmol, 2 equiv) at room temperature. The resulting mixture was stirred for overnight at 60° C. The resulting mixture was diluted with water (100 mL). The aqueous layer was extracted with EtOAc (3×50 mL) The resulting mixture was concentrated under vacuum. The residue was purified by Prep-TLC (DCM/MeOH=15:1) to afford crude product. The crude product (300 mg) was purified by Prep-HPLC with the following conditions (Column: X Bridge Shield RP18 OBD Column, 30*150 mm, 5 μm; Mobile Phase A: water (10 mmol/L NH₄HCO₃), Mobile Phase B: ACN; Flow rate: 60 mL/min; Gradient: 37% B to 57% B in 8 min; Wave Length: 220 nm; RT1 (min): 6.83) to afford 500-1 (200 mg, 34.26%) as a yellow solid

### 2. Synthesis of 500

500-1 (200 mg, 0.369 mmol, 1 equiv) was purified by chiral separation with the following conditions (Column: CHIRALPAK IG, 7*25 cm, 10 μm; Mobile Phase A: Hex (0.5% 2M NH₃-MeOH)-HPLC, Mobile Phase B: EtOH:DCM=1:1-HPLC; Flow rate: 20 mL/min; Gradient: 15% B to 15% B in 19 min; Wave Length: 220/254 nm; RT (min): 12.72; RT2 (min): 15.97; The first peak was the product. Sample Solvent: EtOH:DCM=1:1-HPLC; Injection Volume: 0.7 mL; Number of Runs: 8) to afford 500 (52.9 mg, 25.82%) as a yellow solid.

LC-MS-500 (ES, m/z): [M+H]⁺ 543. H-NMR-500 1H NMR (400 MHz, MeOD ppm) δ 1.40-1.42 (d, 3H), δ 1.75-1.77 (m, 1H), δ 1.80-2.15 (m, 5H), δ 2.22-2.29 (m, 2H), δ 2.41-2.43 (m, 1H), δ 2.65-2.87 (m, 2H), δ 2.02-3.04 (m, 1H), δ 3.26-3.29 (m, 2H), δ 3.48 (s, 3H), δ 4.28-4.31 (d, 1H), δ 5.11-5.25 (m, 1H), δ 7.14 (s, 1H), δ 7.19 (s, 1H), δ 7.26-7.28 (d, 1H), δ 7.48-7.52 (t, 1H), δ 7.64-7.67 (m, 2H), δ 7.71 (s, 1H), δ 8.37 (s, 1H).

### Example 492. Synthesis of Compound 501

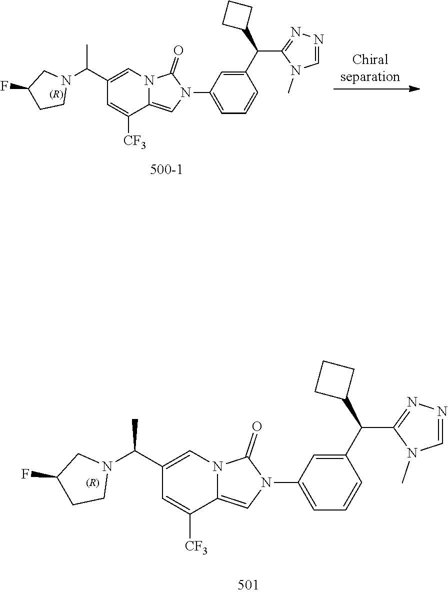

### 1. Synthesis of 501

The 500-1 (200 mg 0,369 mmol, 1 equiv) was purified by chiral separation with the following conditions (Column: CHIRALPAK IG, 7*25 cm, 10 μm; Mobile Phase A: Hex (0.5% 2M NH₃-MeOH)-HPLC, Mobile Phase B: EtOH:DCM=1:1-HPLC; Flow rate: 20 mL/min; Gradient: 15% B to 15% B in 19 min; Wave Length: 220/254 nm; RT1 (min): 12.72; RT2 (min): 15.97; The second peak was the product. Sample Solvent: EtOH:DCM=1:1-HPLC; Injection Volume: 0.7 mL; Number of Runs: 8) to afford 501 (62 m g, 30.66%) as a yellow solid.

LC-MS-501 (ES, m/z): [M+H]⁺ 543. H-NMR-501 1H NMR (400 MHz, MeOD ppm) δ 1.40-1.42 (d, 3H), δ 1.75-1.77 (m, 1H), δ 1.80-2.15 (m, 5H), δ 2.22-2.29 (m, 2H), δ 2.41-2.43 (m, 1H), δ 2.65-2.87 (m, 2H), δ 2.02-3.04 (m, 1H), δ 3.26-3.29 (m, 2H), δ 3.48 (s, 3H), δ 4.28-4.31 (d, 1H), δ 5.11-5.25 (m, 1H), δ 7.14 (s, 1H), δ 7.19 (s, 1H), δ 7.26-7.28 (d, 1H), δ 7.48-7.52 (t, 1H), δ 7.64-7.67 (m, 2H), δ 7.71 (s, 1H), δ 8.37 (s, 1H).

### Example 493. Synthesis of Compound 502

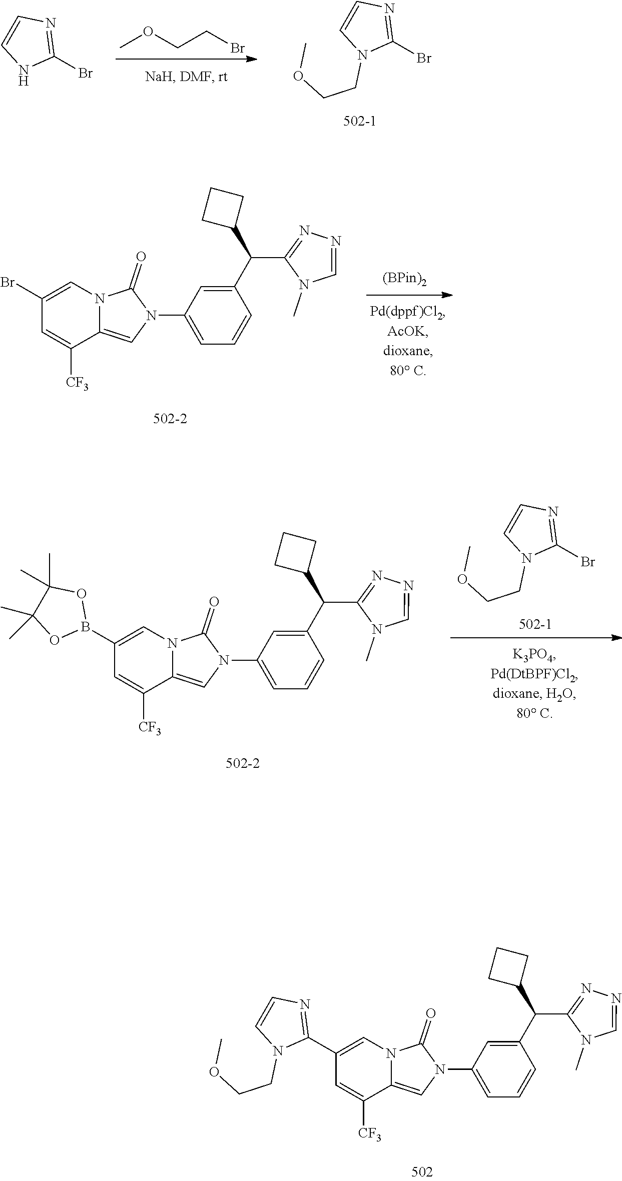

### 1. Synthesis of 502-1

A mixture of 2-bromo-1H-imidazole (5 g, 34,019 mmol, 1 equiv) and NaH (2.04 g, 85.047 mmol, 2.5 equiv) in DMF (50 mL) was stirred for 1 h at 0° C. To the above mixture was added 2-bromoethyl methyl ether (14.19 g, 102057 mmol, 3 equiv) at 0° C. The resulting mixture was stirred for additional overnight at room temperature. The mixture was acidified to pH 7 with saturated NH₄Cl (aq.) (100 mL). The aqueous layer was extracted with EtOAc (3×100 mL). The resulting mixture was concentrated under reduced pressure. The residue was purified by silica gel column chromatography, eluted with CH₂Cl₂/MeOH (200:1) to afford 502-1 (5.5 g, 70.96%) as a yellow oil,

### 2. Synthesis of 502-2

Into a 250 mL round-bottom flask were added 502-1 (1.3 g, 2.568 mmol, 1 equiv) and dioxane (130 mL), KOAc (503.96 mg, 5.136 mmol, 2 equiv) and 4,4,5,5-tetramethyl-2-(tetramethyl-1,3,2-dioxaborolan-2-yl)-1,3,2-dioxaborolane (2.61 g, 10.272 mmol, 4 equiv) and Pd(dppf)Cl₂ (281.80 mg, 0.385 mmol, 0.15 equiv) at room temperature. The resulting mixture was stirred for 8 h at 80° C. under nitrogen atmosphere. The resulting mixture was concentrated under reduced pressure. The residue was purified by silica gel column chromatography, eluted with CH₂Cl₂/MeOH (30:1) to afford 502-2 (750 mg, 47.51%) as a yellow oil.

### 3. Synthesis of 502

To a stirred mixture of 502-2 (750 mg, 3.658 mmol, 2 equiv) and 502-1 (1012.03 mg, 1.829 mmol, 1 equiv) in dioxane (8 mL) H₂O (2 mL) were added K₃PO₄ (1164.56 mg, 5.487 mmol, 3 equiv) and Pd(DtBPF)Cl₂ (178.79 mg, 0,274 mmol, 0.15 equiv) at room temperature under nitrogen atmosphere. The resulting mixture was stirred for 2 h at 80° C. under nitrogen atmosphere. The resulting mixture was concentrated under reduced pressure. The residue was purified by Prep-TLC (CH2Cl2/MeOH 20:1) to afford the crude product. The crude product (350 mg) was purified by Prep-HPLC with the following conditions (Column: YMC-Actus Triart C18 ExRS, 30*150 mm, 5 Lm; Mobile Phase A: Water (10 mmol/L NH₄HCO₃), Mobile Phase B: ACN; Flow rate: 60 mL/min; Gradient: 35% B to 55% B in 7 min; Wave Length: 220 nm; RT1 (min): 5.88) to afford 502 (166.4 mg, 16.40%) as a yellow solid.

LC-MS-502 (ES, m/z): [M+H]⁺ 552. H-NMR-502 (400 MHz, CD₃OD, ppm): δ 1.75-1.81 (m, 1H), 51.88-1.95 (m, 4H), 52.24-2.29 (m, 1H), 53.30-3.35 (m, 1H), 33.38 (s, 3H), 33.56 (s, 3H), 33.78-3.81 (m, 2H), 34.26-4.89 (m, 3H), 37.12-7.13 (d, 1H), 37.23 (s, 1H), 37.27-7.29 (d, 1H), 37.33-7.36 (m, 2H), 37.49-7.54 (m, 1H), 37.66-7.71 (m, 2H), 57.29 (s, 1H), 38.38 (s, 1H).

### Example 494. Synthesis of Compound 503

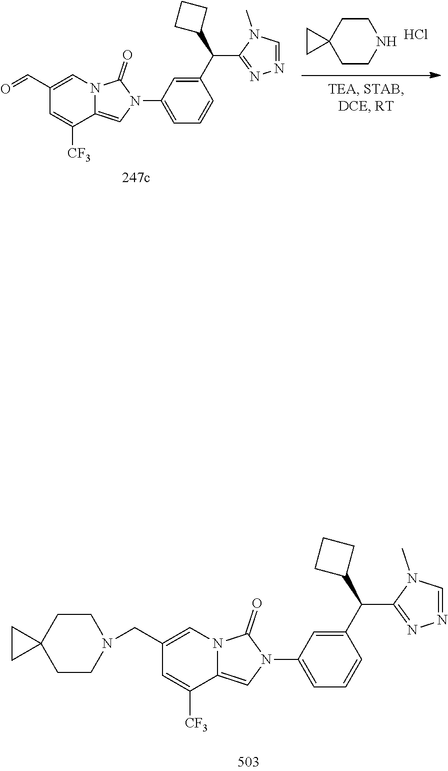

### 1. Synthesis of 503

To a stirred solution of 247c (400 mg, 0.878 mmol, 1 equiv) and 6-azaspiro[2.5]octane hydrochloride (372.28 mg, 1 756 mmol, 2 equiv) in DCE (40 mL) were added TEA (177.75 mg, 1.756 mmol, 2 equiv) at room temperature. The resulting mixture was stirred for 3 h at room temperature. To the above mixture was added STAB (372.28 mg, 1.756 mmol, 2 equiv) at room temperature. The resulting mixture was stirred overnight at room temperature. The reaction was quenched with water (100 ml) at room temperature. The aqueous layer was extracted with DCM (2×50 mL). The resulting mixture was concentrated under vacuum. The residue was purified by reverse flash chromatography with the following conditions: column, C18 silica gel; mobile phase, MeCN in Water (10 mmol/L NH₄HCO3), 30% to 65% gradient in 30 min; detector, UV 254 nm. The resulting mixture was concentrated under reduced pressure. This resulted in 503 (98.8 mg, 20.02%) as a yellow solid.

LC-MS-503 (ES, m/z): [M+H]⁺ 551. H-NMR-503 (400 MHz, DMSO-d6, δ ppm): 0.25 (s, 4H), 1.23-1.34 (d, 4H), 1.68-1.81 (m, 5H), 2.08-2.10 (s, 1H), 2.33-2.67 (m, 4H), 3.21-3.23 (s, 1H), 3.31-3.32 (s, 2H), 3.41-3.43 (s, 3H), 4.24-4.27 (d, 1H), 7.03 (s, 1H), 7.18-7.20 (m, 1H), 7.31 (s, 1H), 7.42-7.46 (m, 1H), 7.67-7.73 (m, 3H), 8.32 (s, 1H).

### Example 495. Synthesis of Compound 504

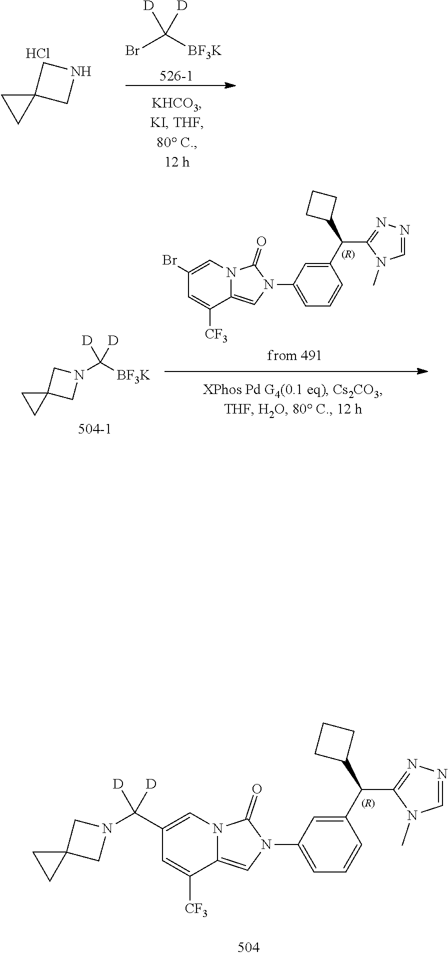

### 1. Synthesis of 504-1

To a solution of [bromo(dideuterio)methyl]-trifluoro-boron;potassium hydride (339 mg, 1.7 mmol, 1.0 equiv) and 5-azaspiro[2.3]hexane (200 mg, 1.7 mmol, 1.0 equiv, HC) in THF (4.0 mL) was added KHCO₃ (334 mg, 3.4 mmol, 2.0 equiv) and KI (27 mg, 0.1 equiv). The mixture was stirred at 80° C. for 12 hours. The reaction mixture was concentrated in vacuum. The residue was triturated with acetone (5 mL) at 35° C. for 1 hour. Then the mixture was filtered and the filtrate was concentrated in vacuum to afford 504-1 (270 mg, 78.73% yield) was obtained as colorless oil.

### 2. Synthesis of 504

A mixture of 504-1 (170 mg, 2.0 equiv), 6-bromo-2-[3-[(R)-cyclobutyl-(4-methyl-1,2,4-triazol-3-yl)methyl]phenyl]-8-(trifluoromethyl)imidazo[1,5-a]pyridin-3-one (210 mg, 1.0 equiv), Cs2CO3 (405 mg, 1.2 mmol, 3.0 equiv), dicyclohexyl-[2-(2,4,6-triisopropylphenyl)phenyl]phosphane; methanesulfonate;[2-[2-(methylamino)phenyl]phenyl]palladium(1+) (35 mg, 0.1 equiv) in THF (10 mL) and H2O (2 mL) was degassed and purged with N₂ for 3 times, and then the mixture was stirred at 80° C. for 12 hours under N₂ atmosphere. The reaction was poured into water (20 mL) and the resulting mixture was extracted with EtOAc (2×15 mL). The organic phase was washed with brine (10 mL), dried over anhydrous Na2SO4, concentrated in vacuum to give a residue. The residue was purified by prep-HPLC (Column: Phenomenex Luna C18 75*30 mm*3 um; Mobile Phase A: Water (0.2% formic acid), Mobile Phase B: ACN; Flow rate: 25 mL/min; Gradient: 20% B to 60% B in 8 min; Wave Length: 220 nm; RT1 (min): 8.3) to afford 504 (62 mg, 28.41% yield) as a yellow solid.

MS-504: (ES, m/z): [M+H]+ 525.2. H-NMR-504: (400 MHz, METHANOL-d4) δ ppm 8.42 (s, 1H), 8.35 (s, 1H), 7.89 (s, 1H), 7.65-7.59 (m, 2H), 7.52-7.45 (m, 1H), 7.28 (d, J=2.4 Hz 1H), 7.17 (s, 1H), 7.08 (s, 1H), 4.28 (d, J=3.2 Hz, 1H), 3.92 (s, 4H), 3.55 (s, 3H), 3.30-3.23 (m, 1H), 2.30-2.19 (m, 1H), 1.96-1.83 (m, 4H), 1.82-1.71 (m, 1H), 0.73 (s, 4H).

### Example 496. Synthesis of Compound 505

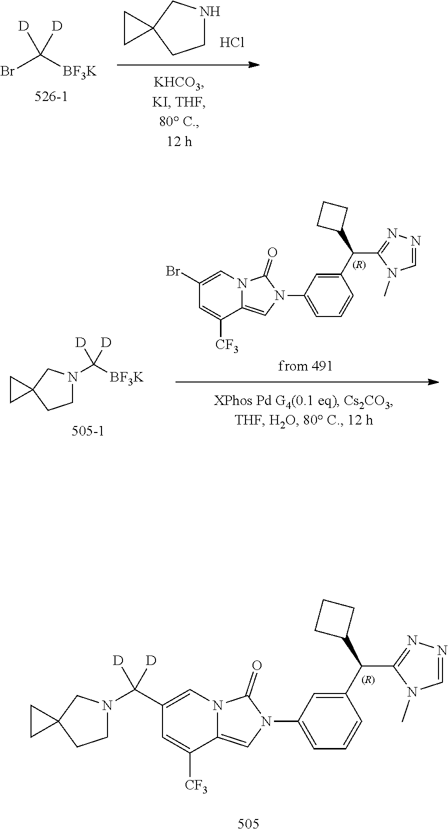

### 1. Synthesis of 505-1

A mixture of 526-1 (303.62 mg, 1.50 mmol, 1 equiv), 5-azaspiro[2.4]heptane (200 mg, 1.50 mmol, 1 equiv, HC), KHCO₃ (299.70 mg, 2.99 mmol, 2 equiv), K1 (24.85 mg, 149.68 umol, 0.1 equiv) in THF (4 mil) and then the mixture was stirred at 80° C. for 12 hr under N atmosphere. The reaction was concentrated in reduced pressure, then the residue was triturated with acetone (20 mL) at 35° C. for 1 hr. Then the mixture was filtered and the filtrate was concentrated in vacuum to afford 505-1 (0.28 g, 85%) as a yellow oil. NN

### 2. Synthesis of 505

To a solution of 505-1 (0.22 g, 1.00 mmol, 1 equiv) in THF (5 mL)/H₂O (1 ml) was added Xphos Pd G4 (86.40 mg, 100.41 umol, 0.1 equiv), 6-bromo-2-[3-[(R)-cyclobutyl-4-methyl-1,2,4-triazol-3-yl)methyl]phenyl]-8-(trifluoromethyl)imidazo[1,5-a]pyridin-3-one (305.05 mg, 602.48 umol, 0.6 equiv) and Cs₂CO₃ (981.51 mg, 3.01 mmol, 3 equiv). The resulting mixture was stirred at 80° C. for 12 hr. The reaction was poured into water (10 mL) and the resulting mixture was extracted with EtOAc (10 mL×2). The organic phase was washed with brine (10 mL), dried over anhydrous Na₂SO₄, concentrated in vacuum to give a residue. The residue was purified by normal phase SiO₂ chromatography (10-100% MeOH/DCM) to give crude product. The crude product (157 mg) was purified by Prep-HPLC with the following conditions (column: Phenomenex Luna C18 200*40 mm*10 um; Mobile phase A: Water (FA), Mobile phase B: ACN; Flow rate: 50 mL/min; Gradient: 1% B to 30% B in 8 min; Wave Length: 220 nm) to afford 505 (79.1 mg, 49.8%) as a yellow solid.

MS-505: (ES, m/z): [M+H]+ 539.27. H-NMR-505: (400 MHz, METHANOL-d4) δ 8.35 (s, 1H), 7.84-7.75 (m, 1H), 7.69-7.58 (m, 2H), 7.54-7.43 (m, 1H), 7.32-7.22 (m, 1H), 7.20-7.07 (m, 2H), 4.33-4.22 (m, 1H), 3.53 (s, 3H), 3.30-3.25 (m, 1H), 3.11-2.98 (m, 2H), 2.80 (s, 2H), 2.31-2.18 (m, 1H), 2.01-1.67 (m, 7H), 0.70-0.59 (m, 4H).

### Example 497. Synthesis of Compound 506_P1&P2

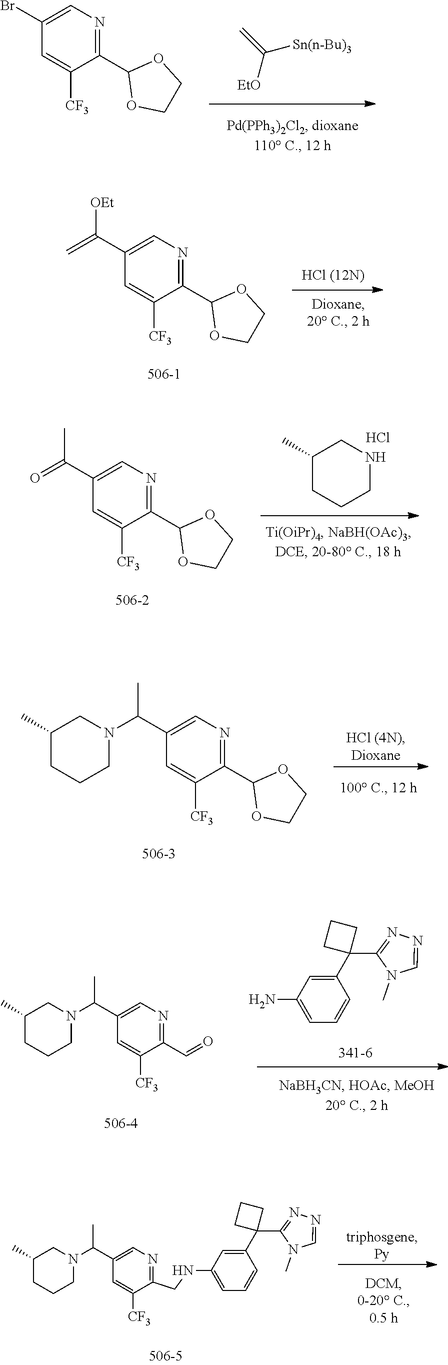

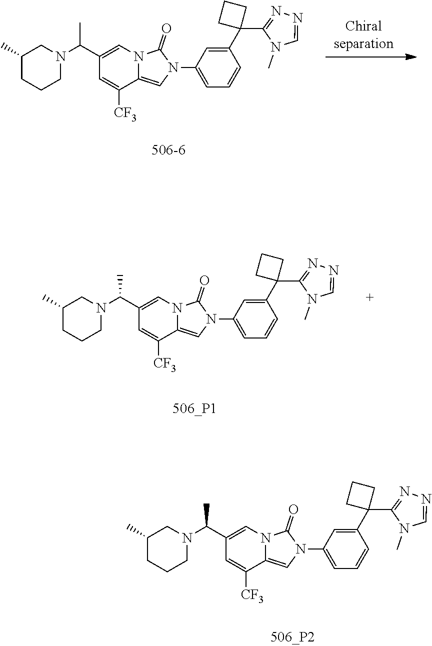 [SMILES: CC(c1cc(C(F)(F)F)c2cn(-c3cccc(C4(c5nncn5C)CCC4)c3)c(=O)n2c1)N1CCC[C@H](C)C1 ; C[C@H]1CCCN([C@H](C)c2cc(C(F)(F)F)c3cn(-c4cccc(C5(c6nncn6C)CCC5)c4)c(=O)n3c2)C1 ; C[C@H]1CCCN([C@@H](C)c2cc(C(F)(F)F)c3cn(-c4cccc(C5(c6nncn6C)CCC5)c4)c(=O)n3c2)C1]

### 1. Synthesis of 506-1

A mixture of 5-bromo-2-(1,3-dioxolan-2-yl)-3-(trifluoromethyl)pyridine (5 g, 16.78 mmol, 1 equiv), tributyl(1-ethoxyvinyl)stannane (6.23 mL, 18.4 mmol, 1.1 equiv) and Pd(PPh₃)Cl₂ (589 mg, 839 umol, 0.05 equiv) in dioxane (100 mL) was stirred at 110° C. for 12 h under nitrogen atmosphere. The reaction mixture was quenched by addition Sat. KF (aq. 400 mL), and then diluted with water (100 mL) and extracted with EtOAc (150 mL×3). The combined organic layers were dried over Na₂SO₄, filtered and the filtrate was concentrated under reduced pressure to give a residue. The residue was purified by silica gel column chromatography eluted with PE/EtOAc (5:1) to afford 506-1 (4.4 g, 90%) as a yellow oil.

¹H-NMR-506-1: (400 MHz, CHLOROFORM-d) δ 8.97 (d, J=1.6 Hz, 1H), 8.09 (d, J=2.0 Hz, 1H), 6.21 (s, 1H), 4.71 (d, J=3.2 Hz, 1H), 4.34-4.24 (m, 3H), 4.07-4.01 (m, 2H), 3.92-3.84 (m, 2H), 1.36 (t, J=7.2 Hz, 3H)

### 2. Synthesis of 506-2

To a solution of 506-1 (4.4 g, 15.21 mmol, 1 equiv) in HCl (017 mL) and dioxane (45 mL). The mixture was stirred at 20° C. for 2 h. The reaction mixture was adjusted to pH=8 with sat.NaHCO₃ (600 mL) and extracted with EtOAc (200 mL×3). The combined organic layers were dried over anhydrous Na₂SO₄, filtered and the filtrate was concentrated under reduced pressure to give a residue. The residue was purified by silica gel column chromatography eluted with PE/EtOAc (3:1) to afford 506-2 (3.8 g, 95%) as a yellow oil.

### 3. Synthesis of 506-3

To a solution of 506-2 (3.8 g, 14.5 mmol, 1 equiv) and (3S)-3-methylpiperidine (2.96 g, 21.8 mmol, 1.5 equiv, HCl) in DCE (70 mL) was added tetraisopropoxytitanium (17.2 mL, 58.2 mmol, 4 equiv). The mixture was stirred at 80° C. for 12 h under nitrogen atmosphere. Then NaBH(OAc)₃ (9.25 g, 43.6 mmol, 3 equiv) was added at 20° C. The mixture was stirred at 80° C. for 6 h under nitrogen atmosphere. The mixture was adjust to pH=8 by sat.NaHCO₃ (300 ml) and extracted with EtOAc (100 ml×3). The combined organic layers were dried over anhydrous Na₂SO₄, filtered and the filtrate was concentrated under reduced pressure. The residue was purified by silica gel column chromatography eluted with DCM/MeOH (10:1) to afford 506-3 (3.2 g, 63%) as a yellow oil.

¹H-NMR-506-3: (400 MHz, CHLOROFORM-d) δ 8.72 (s, 1H), 7.87 (d, J=1.2 Hz, 1H), 6.18 (s, 1H), 4.33-4.23 (m, 2H), 4.07-4.03 (m, 2H), 3.53-3.43 (m, 1H), 2.84-2.67 (m, 1H), 2.62-2.48 (m, 1H), 1.88-1.77 (m, 1H), 1.62-1.41 (m, 5H), 1.32-1.25 (m, 3H), 0.80-0.70 (m, 4H)

### 4. Synthesis of 506-4

To a solution of 506-3 (3.2 g, 9.29 mmol, 1 equiv) in HCl (30 mL) and dioxane (30 mL) was stirred at 100° C. for 12 h. The mixture was adjust to pH=8 by sat.NaHCO3 (200 ml) and extracted with EtOAc (100 ml×3). The combined organic layers were dried over anhydrous Na₂SO₄, filtered and the filtrate was concentrated under reduced pressure. The residue was purified by silica gel column chromatography eluted with DCM/MeOH (10:1) to afford 506-4 (2.1 g, 75%) as a yellow oil.

¹H-NMR-506-4: (400 MHz, CHLOROFORM-d) δ 10.17 (s, 1H), 8.86 (s, 1H), 8.06 (s, 1H), 3.52 (s, 1H), 2.81-2.68 (m, 1H), 2.59-2.49 (m, 1H), 1.96-1.83 (m, 1H), 1.63-1.55 (m, 4H), 1.36-1.30 (m, 3H), 0.82-0.71 (m, 5H)

### 5. Synthesis of 506-5

To a solution of 506-4 (0.9 g, 3.0 mmol, 1 equiv) and 341-6 (684 mg, 3.0 mmol, 1 equiv) in MeOH (15 mL) was added HOAc (514 uL, 8.99 mmol, 3 equiv). The mixture was stirred at 20° C. for 1 h. Then NaBH₃CN (376 mg, 5.99 mmol, 2 equiv) was added. The mixture was stirred at 20° C. for 1 h under nitrogen atmosphere. The mixture was adjust to pH=8 by Sat.NaHCO₃ (150 ml) and extracted with DCM (50 ml×3). The combined organic layers were dried over anhydrous Na₂SO₄, filtered and the filtrate was concentrated under reduced pressure. The residue was purified by silica gel column chromatography eluted with DCM/MeOH (10:1) to afford 506-5 (0.9 g, 58%) as a yellow solid.

### 6. Synthesis of 506-6

To a solution of 506-5 (370 mg, 721 umol, 1 equiv) in DCM (5 mL) was added Py (349 uL, 4.33 mmol, 6 equiv) and bis(trichloromethyl) carbonate (107 mg, 361 umol, 0.5 equiv) at 0° C. The mixture was stirred at 20° C. for 0.5 h under nitrogen atmosphere. The same scale reaction was conducted in parallel for 3 batches in total and work up together. The reaction was quenched with sat.NaHCO₃ (100 mL). The aqueous layer was extracted with DCM (50 mL×3). The combined organic layers were dried over anhydrous Na₂SO₄, filtered and the filtrate was concentrated in vacuum. The residue was purified by silica gel column chromatography elated with EtOAc/MeOH (10:1) to afford 506-6 (0.9 g, 35.5%) as a yellow solid.

### 7. Synthesis of 506-P1&P2

The 506-6 (0.9 g) was purified by Chiral separation with the following conditions (column: REGIS(S,S)WHELK-O1 (250 mm*30 mm, 10 um); mobile phase: [Neu-ETOH]; B %: 27%-27%, 6 min, Flow rate: 55 mL/min; Wave Length: 220/254 nm; RT1 (min): 471; RT2 (min): 6.03) to afford 506_P1 (206 mg, 26%) as a yellow solid and 506_P2 (239.6 mg, 30%) as a yellow solid.

MS-506_P1: (ES, m/z): [M+H]⁺ 539.3 ¹H-NMR-506_P1: (400 MHz, METHANOL-d4) δ 8.35 (s, 1H), 7.76 (t, J=1.6 Hz, 1H), 7.60 (s, 2H), 7.57-7.51 (m, 1H), 7.32-7.27 (m, 1H), 7.15 (d, J=12.0 Hz, 2H), 3.42-3.36 (m, 1H), 3.33 (s, 3H), 3.05-2.98 (m, 2H), 2.94 (d, J=10.8 Hz, 1H), 2.85-2.78 (m, 3H), 2.21-2.08 (m, 2H), 2.02-1.94 (m, 1H), 1.75-1.67 (m, 3H), 1.66-1.52 (m, 2H), 1.36 (d, J=6.8 Hz, 3H), 0.96-0.88 (m, 1H), 0.86 (d, J=6.4 Hz, 3H)

MS-506-P2: (ES, m/z): [M+H]⁺ 539.3 ¹H-NMR-506_P2: (400 MHz, METHANOL-d4) δ 8.36 (s, 1H), 7.76 (t, J=1.6 Hz, 1H), 7.63-7.57 (m, 2H), 7.56-7.52 (m, 1H), 7.29 (d, J=7.6 Hz, 1H), 7.15 (d, J=10.4 Hz, 2H), 3.42-3.37 (m, 1H), 3.33 (s, 3H), 3.05-2.98 (m, 2H), 2.89 (d, J=8.0 Hz, 1H), 2.87-2.78 (m, 3H), 2.20-2.02 (m, 3H), 1.75-1.65 (m, 4H), 1.60-1.53 (m, 1H), 1.37 (d, J=6.8 Hz, 3H), 0.92-0.86 (m, 4H).

### Example 498. Synthesis of Compound 507

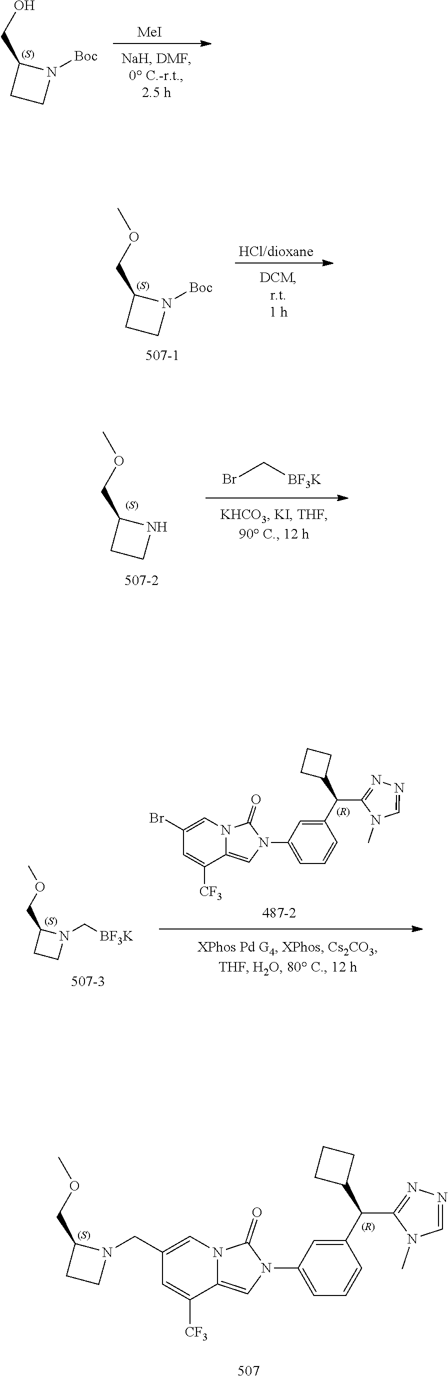

### 1. Synthesis of 507-1

A solution of NaH (469.14 mg, 60% purity, 1.22 equiv) in DMF (10 mL) was cooled to 0° C. under nitrogen atmosphere. Then a solution of tert-butyl (2S)-2-(hydroxymethyl) azetidine-1-carboxylate (1.8 g, 9.61 mmol, 1 equiv) in DMF (5 mL) was added to the mixture dropwise. The mixture was stirred at 0° C. for 0.5 hr. Then iodomethane (897 uL, 14.4 mmol, 1.5 equiv) was added to reaction mixture at 0° C. The mixture was stirred at 20° C. for 2 hr. The mixture was quenched by saturated NH₄Cl (50 ml) and extracted with EtOAc (3×50 ml). The combined organic layers were dried over anhydrous Na₂SO₄, filtered and the filtrate was concentrated under reduced pressure. The residue was purified by silica gel chromatography eluted with PE/EA=1:1 to afford 507-1 (1.45 g, 75%) as a white oil.

¹H-NMR-507-1: (400 MHz, CHLOROFORM-d) δ 4.24 (s, 1H), 3.94-2.95 (m, 7H), 2.13 (s, 2H), 1.38 (s, 9H)

### 2. Synthesis of 507-2

To a solution of 507-1 (1.13 g, 5.61 mmol, 1 equiv) in DCM (10 mL) was added HCl/dioxane (4 M, 8.42 mL, 6 equiv). The mixture was stirred at 20° C. for 1 hr. The reaction mixture was concentrated at room temperature to afford 507-2 (560 mg, crude) as a white oil.

### 3. Synthesis of 507-3

A mixture of 507-2 (560 mg, 5.54 mmol, 1 equiv), potassium; bromomethyl (trifluoro) boranuide (1.11 g, 5.54 mmol, 1 equiv) KI (91.9 mg, 553 umol, 0.1 equiv), KHCO₃ (1.11 g, 11.1 mmol, 2 equiv) in THF (8 mL) was degassed and purged with nitrogen for 3 times, and then the mixture was stirred at 90° C. for 12 h under nitrogen atmosphere. The reaction mixture was concentrated under reduced pressure to give a residue. The crude product was triturated with acetone at 40° C. for 60 min. Then the mixture was filtered and the filtrate was concentrated in vacuum to afford 507-3 (1.2 g, crude) as a yellow solid.

### 4. Synthesis of 507

To a solution of 507-3 (655 mg, 2.96 mmol, 5 equiv) and 487-2 (300 mg, 592 umol, 1 equiv) in THF (4 mL) and water (1 mL) was added XPhos (28.2 mg, 59.25 umol, 0.1 equiv) and Cs₂CO₃ (579 mg, 1.78 mmol, 3 equiv), dicyclohexyl-[2-(2,4,6-triisopropylphenyl)phenyl]phosphane; methanesulfonate;[2-[2-(methylamino)phenyl]phenyl]palladium(1+) (51 mg, 59.2 umol, 0.1 equiv). The mixture was stirred at 80° C. for 12 h under nitrogen atmosphere. The reaction mixture was diluted with water 10 mL and extracted with EtOAc (10 mL×3). The combined organic layers were dried over anhydrous Na₂SO₄, filtered and concentrated under reduced pressure to give a residue. The residue was purified by flash silica gel chromatography, eluted with CH₂Cl₂/MeOH=10:1 to give crude product. The crude product (230 mg) was purified by Prep-HPLC with the following conditions (Column: Phenomenex Luna C18 200*40 mm*10 um; Mobile phase A: Water (0.2% formic acid), B: ACN; Flow rate: 50 ml/min Gradient: 1% B to 50% B in 8 min; Wave Length: 220 um; RT1 (min): 7.5 min) to afford 507 (41 mg, 13%) as a yellow solid.

MS-507: (ES, m/z): [M+H]⁺ 541.1. ¹H-NMR-507 (400 MHz, ACETONITRILE-d₃) S 8.08 (s, 1H), 7.71-7.60 (m, 2H), 7.56 (s, 1H), 7.45 (t, J=7.6 Hz, 1H), 7.24 (d, J=7.6 Hz, 1H), 6.99 (s, 1H), 6.96 (s, 1H), 4.16 (d, J=10.4 Hz, 1H), 3.53 (d, J=13.2 Hz, 1H), 3.42 (s, 3H), 3.41-3.36 (m, 2H), 3.35-3.27 (m, 4H), 3.26 (s, 3H), 2.96-2.87 (m, 1H), 2.24-2.16 (m, 1H), 2.08-2.00 (m, 1H), 1.95-1.82 (m, 5H), 1.80-1.72 (m, 1H).

### Example 499. Synthesis of Compound 508

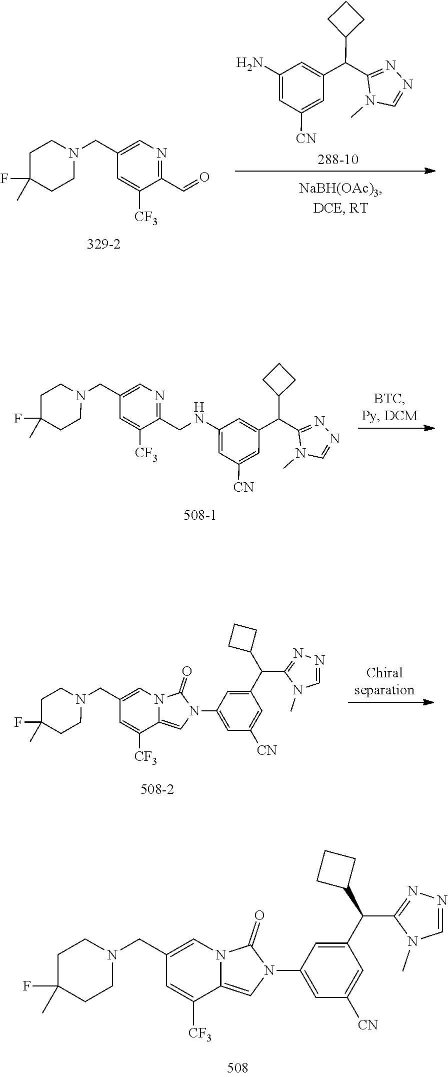 [SMILES: Cn1cnnc1C(c1cc(N)cc(C#N)c1)C1CCC1 ; CC1(F)CCN(Cc2cnc(C=O)c(C(F)(F)F)c2)CC1 ; Cn1cnnc1C(c1cc(C#N)cc(NCc2ncc(CN3CCC(C)(F)CC3)cc2C(F)(F)F)c1)C1CCC1 ; Cn1cnnc1C(c1cc(C#N)cc(-n2cc3c(C(F)(F)F)cc(CN4CCC(C)(F)CC4)cn3c2=O)c1)C1CCC1 ; Cn1cnnc1[C@@H](c1cc(C#N)cc(-n2cc3c(C(F)(F)F)cc(CN4CCC(C)(F)CC4)cn3c2=O)c1)C1CCC1]

### 1. Synthesis of 508-1

A solution of 329-2 (1001.64 mg, 3,291 mmol, 1.1 equiv) and 288-10 (800 mg, 2.992 mmol, 1.00 equiv) in DCE (10 mL) was stirred overnight at room temperature. To the above mixture was added STAB (1268.46 mg, 5984 mmol, 2 equiv) at room temperature. The resulting mixture was stirred for 4 h at room temperature. The reaction was quenched with saturated NaHCO3 (aq.) (80 mL) at room temperature. The resulting mixture was extracted with CH₂Cl₂ (3×50 mL). The resulting mixture was concentrated under reduced pressure. The residue was purified by Prep-TLC (CH₂Cl₂/MeOH 20:1) to afford 508-1 (650 mg, 37.14%) as a light yellow oil.

### 2. Synthesis of 508-2

To a stirred solution of 508-1 (650 mg, 1.170 mmol, 1 equiv) and Pyridine (555.21 mg, 7.020 mmol, 6 equiv) in DCM (10 mL) were added Triphosgene (121.50 mg, 0.409 mmol, 0.35 equiv) at 0° C. The resulting mixture was stirred for 30 min at room temperature. The reaction was quenched with saturated NaHCO₃ (aq.) (60 mL) at room temperature. The resulting mixture was extracted with CH2Cl₂ (3×40 mL). The resulting mixture was concentrated under reduced pressure. The residue was purified by Prep-TLC (Cl₂Cl₂/MeOH 10:1) to afford 508-2 (300 mg, 43.65%) as a yellow solid.

### 3. Synthesis of 508

The 508-2 (300 mg) was purified by Chiral separation with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 10.5 min; Wave Length: 220/254 nm; RT1 (min): 5.87; RT2 (min): 8.06; the first peak is product) to afford 508 (114.0 mg, 37.16%) as a yellow solid.

LC-MS-508 (ES, m/z): [M+H]⁺ 582. H-NMR-508 (400 MHz, DMSO-d6, δ ppm): 1.25-1.39 (d, 3H), 1.52-1.60 (m, 1H), 1.61-1.85 (m, 8H), 2.07-2.08 (m, 1H), 2.23-2.31 (m, 1H), 2.67-2.68 (m, 2H), 3.18-3.22 (m, 1H), 3.32 (s, 2H), 3.48 (s, 3H), 4.38-4.40 (d, 1H), 7.04 (s, 1H), 7.52 (s, 1H), 7.70-7.75 (d, 2H), 8.23-8.24 (d, 2H), 8.37 (s, 1H).

### Example 500. Synthesis of Compound 509

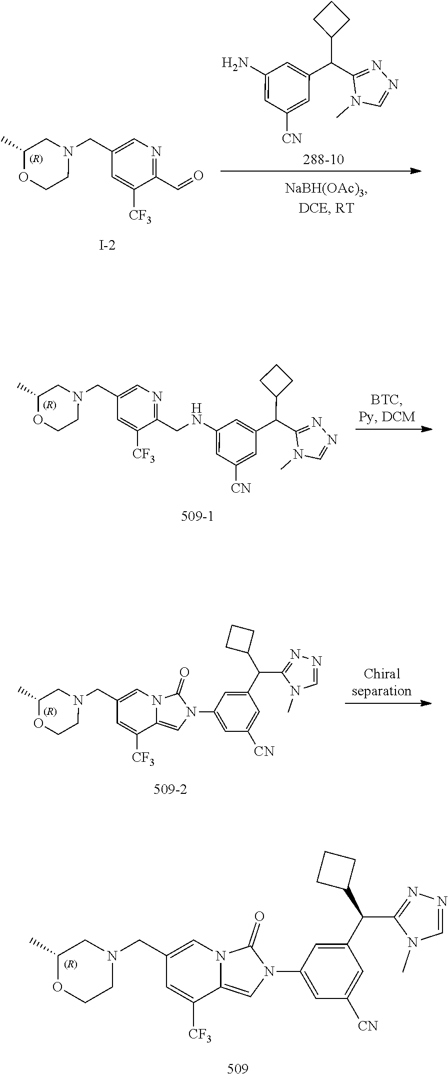

### 1. Synthesis of 509-1

A solution of 288-10 (500 mg, 1,870 mmol, 1.00 equiv) and 1-2 (593.07 mg, 2,057 mmol, 1.1 equiv) in DCE (10 mL) was stirred overnight at room temperature. To the above mixture was added STAB (792.79 mg 3.740 mmol, 2 equiv) at room temperature. The resulting mixture was stirred for 4 h at room temperature. The reaction was quenched with saturated NaHCO₃ (aq.) (60 mL) at room temperature. The resulting mixture was extracted with CH₂Cl₂ (3×50 mL). The resulting mixture was concentrated under reduced pressure. The residue was purified by Prep-TLC (CH₂Cl₂/MeOH 20:1) to afford 509-1 (470 mg, 43.313%) as a light yellow solid.

### 2. Synthesis of 509-2

To a stirred solution of 509-1 (470 mg 0.871 mmol, 1 equiv) and Pyridine (413.38 mg, 5.226 mmol, 6 equiv) in DCM (10 mL) were added Triphosgene (90.46 mg, 0.305 mmol, 0.35 equiv) at 0° C. The resulting mixture was stirred for 10 min at room temperature. The reaction was quenched with saturated NaHCO₃ (aq.) (80 mL) at room temperature. The resulting mixture was extracted with CH₂Cl₂ (3×30 mL). The resulting mixture was concentrated under reduced pressure. The residue was purified by reverse flash chromatography with the following conditions: column, C18 silica gel; mobile phase, MeCN in Water (10 mmol/L NH₄HCO₃), 30% to 50% gradient in 10 min; detector, UV 254 nm. This resulted in 509-2 (260 mg, 49.08%) as a yellow solid.

### 3. Synthesis of 509

The 509-2 (260 mg) was purified by Chiral separation with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 11 min; Wave Length: 220/254 nm; RT1 (min): 6.67; RT2 (min): 8.16; the first peak is product) to afford 509 (92.6 mg, 35.15%) as a yellow solid.

LC-MS-509 (ES, m/z): [M+H]⁺ 566. H-NMR-509 (400 MHz, DMSO-d6, δ ppm): 1.00-1.10 (d, 3H), 1.63-1.85 (m, 6H), 2.03-2.11 (m, 2H), 2.61-2.75 (m, 2H), 3.18-3.30 (m, 3H), 3.41-3.53 (m, 5H), 3.73-3.75 (d, 1H), 4.35-4.40 (m, 1H), 7.04 (s, 1H), 7.52 (s, 1H), 7.68-7.70 (d, 2H), 8.23-8.24 (d, 2H), 8.37 (s, 1H).

### Example 501. Synthesis of Compound 510

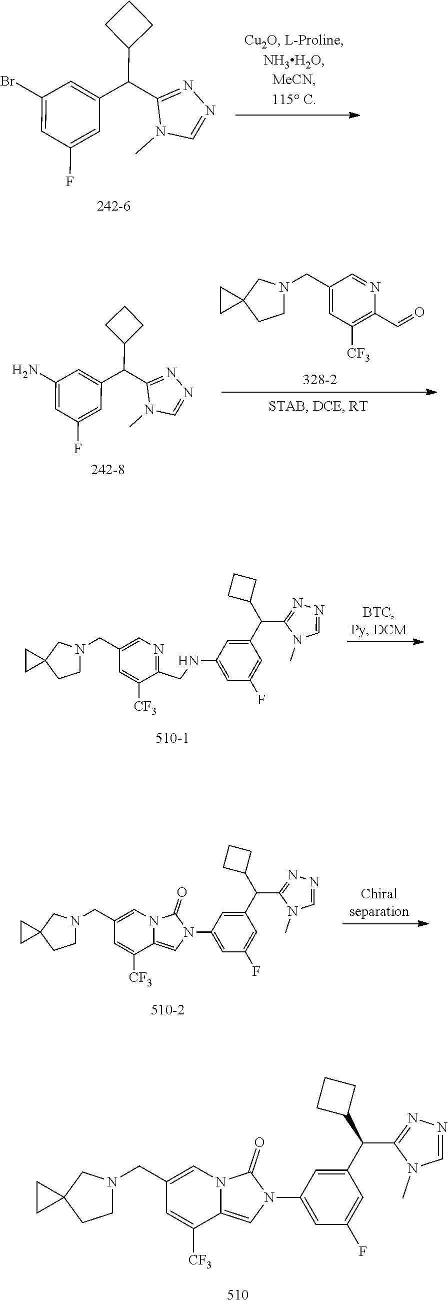 [SMILES: Cn1cnnc1C(c1cc(F)cc(Br)c1)C1CCC1 ; Cn1cnnc1C(c1cc(N)cc(F)c1)C1CCC1 ; O=Cc1ncc(CN2CCC3(CC3)C2)cc1C(F)(F)F ; Cn1cnnc1C(c1cc(F)cc(NCc2ncc(CN3CCC4(CC4)C3)cc2C(F)(F)F)c1)C1CCC1 ; Cn1cnnc1C(c1cc(F)cc(-n2cc3c(C(F)(F)F)cc(CN4CCC5(CC5)C4)cn3c2=O)c1)C1CCC1 ; Cn1cnnc1[C@@H](c1cc(F)cc(-n2cc3c(C(F)(F)F)cc(CN4CCC5(CC5)C4)cn3c2=O)c1)C1CCC1]

### 1. Synthesis of 242-8

Into a pressure tank reactor was added 242-6 (10 g, 30.845 mmol, 1 equiv), L-Proline (0.07 g, 0.617 mmol, 0.1 equiv) and Cu₂O (0.88 g, 6,150 mmol, 0.20 equiv) in NH₄OH (150 mL) and MeCN (150 mL) at room temperature. The resulting mixture was stirred overnight at room temperature. The resulting mixture was concentrated under vacuum. The residue was purified by silica gel column chromatography, eluted with DCM/MeOH (50:1) to afford 242-8 (4.7 g, 52.68%) as a yellow solid,

### 2. Synthesis of 510-1

To a stirred solution/mixture of 242-8 (600 mg, 2.305 mmol, 1 equiv) and 328-2 (786.29 mg, 2.766 mmol, 1.2 equiv) in DCE (6 mL) at room temperature. The resulting mixture was stirred overnight at room temperature. To the above mixture was added STAB (977.00 mg 4.610 mmol, 2 equiv) at room temperature. The resulting mixture was stirred for additional 4 h at room temperature. The reaction was quenched with saturated NaHCO₃ (aq.) (80 mL) at room temperature. The aqueous layer was extracted with CH₂Cl₂ (3×30 mL). The residue was purified by Prep-TLC (CH₂Cl₂/MeOH 17:1) to afford 510-3 600 mg, 49.25%) as a white solid.

### 3. Synthesis of 510-2

To a stirred mixture of 510-1 (600 mg, 1.135 mmol, 1 equiv) and Pyridine (538.71 mg, 6.810 mmol, 6 equiv) in DCM (6 mL) was added Triphosgene (131.36 mg, 0.443 mmol, 0.39 equiv) at room temperature. The resulting mixture was stirred for 10 min at room temperature. The reaction was quenched with saturated NaHCO₃ (aq.) (80 mL) at room temperature. The aqueous layer was extracted with DCM (3×40 mL). The resulting mixture was concentrated under vacuum. The residue was purified by Prep-TLC (CH2Cl₂/MeOH 15:1) to afford 510-4 (320 mg, 50.83%) as a yellow solid.

### 4. Synthesis of 510-0

The 510-4 (320 mg) was purified by Prep-Chiral-HPLC with the following conditions (Column: CHIRALPAK IF, 2*25 cm, 5 um; Mobile Phase A: Hex (0.5% 2M NH₃-MeOH), Mobile Phase B: EtOH:DCM=1:1; Flow rate: 20 mL/min; Gradient: 30% B to 30% B in 10 min; Wave Length: 220/254 nm; RT1 (min): 7.24; RT2 (min): 8.45; the second peak is product) to afford 510 (114.3 mg, 35.71%) as a yellow solid.

LC-MS-510 (ES, m/z): [M+H]⁺ 555. H-NMR-510 (400 MHz, DMSO-d₆, δ ppm): 0.48-0.58 (d, 4H), 1.68-1.82 (m, 7H), 2.06-2.08 (m, 1H), 2.45-2.50 (m, 2H), 2.63-2.73 (m, 2H), 3.18-3.24 (m, 1H), 3.39 (s, 2H), 3.46 (s, 3H), 4.30-4.33 (d, 1H), 7.04-7.06 (m, 2H), 7.42 (s, 1H), 7.69-7.72 (m, 3H), 8.35 (s, 1H).

### Example 502. Synthesis of Compound 511

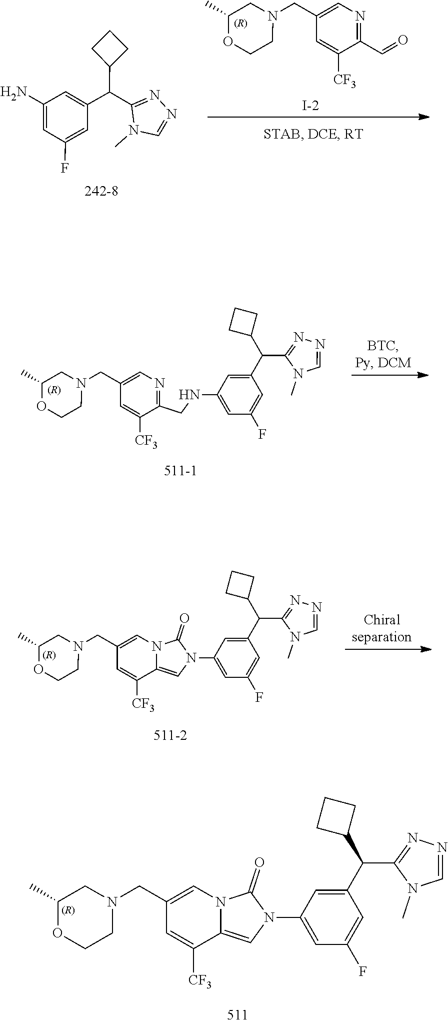

### 1. Synthesis of 511-1

To a stirred solution of 242-8 (500 mg, 1.921 mmol, 1.00 equiv) in DCE (20 mL) was added 1-2 (664.43 mg, 2.305 mmol, 1.2 equiv) at room temperature. The resulting mixture was stirred for overnight at room temperature. To the above mixture was added STAB (814.16 ng, 3.842 mmol, 2 equiv) at room temperature. The resulting mixture was stirred for additional 6 h at room temperature. The reaction was quenched with saturated NaHCO₃ (aq.) at room temperature. The aqueous layer was extracted with DCM (3×30 mL). The resulting mixture was concentrated under vacuum. The residue was purified by Prep-TLC (DCM/MeOH 18:1) to afford 511-1 (500 mg, 46.43%) as a white solid.

### 2. Synthesis of 511-2

To a stirred solution of 511-1 (480 mg, 0.901 mmol, 1 equiv) and Pyridine (712.90 mg 9.010 mmol, 10 equiv) in DCM (10 mL) was added Triphosgene (106.97 mg, 0.360 mmol, 0.4 equiv) at room temperature. The resulting mixture was stirred for 10 min at room temperature. The reaction was quenched with saturated NaHCO₃ (aq.) at room temperature. The aqueous layer was extracted with DCM (2×30 mL). The resulting mixture was concentrated under vacuum. The residue was purified by Prep-TLC (DCM/MeOH 20:1) to afford 511-2 (350 mg, 66.74%) as a yellow solid.

### 3. Synthesis of 511

The 511-2 (350 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 10 min; Wave Length: 220/254 min; RT1 (min): 5.61; RT2 (min): 8.01; the second peak is product) to afford 511 (124.2 mg, 35.49%) as a yellow solid.

LC-MS-511 (ES, m/z): [M+H]⁺ 559. H-NMR-511 (400 MHz, DMSO-d6, δ ppm): 1.03-1.04 (d, 3H), 1.70-1.84 (m, 6H), 2.03-2.12 (m, 2H), 2.65-2.67 (m, 1H), 2.72-2.74 (m, 1H), 3.20-3.27 (m, 1H), 3.27 (s, 2H), 3.45-3.51 (m, 5H), 3.72-3.75 (m, 1H), 4.30-4.33 (d, 1H), 7.03-7.06 (m, 2H), 7.41 (s, 1H), 7.68-7.71 (m, 3H), 8.35 (s, 1H).

### Example 503. Synthesis of Compound 512

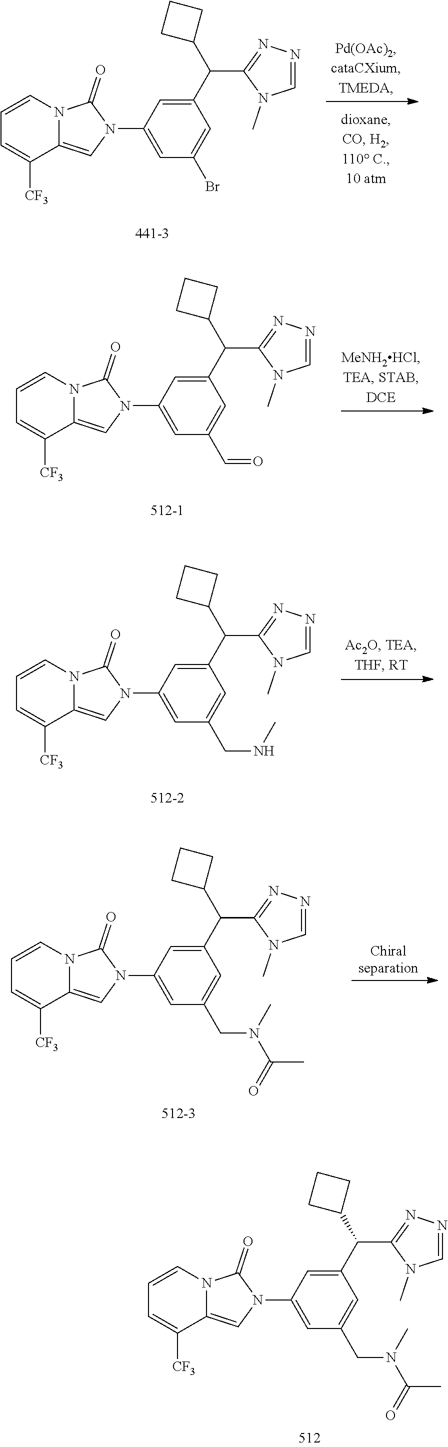 [SMILES: Cn1cnnc1C(c1cc(Br)cc(-n2cc3c(C(F)(F)F)cccn3c2=O)c1)C1CCC1 ; Cn1cnnc1C(c1cc(C=O)cc(-n2cc3c(C(F)(F)F)cccn3c2=O)c1)C1CCC1 ; CNCc1cc(C(c2nncn2C)C2CCC2)cc(-n2cc3c(C(F)(F)F)cccn3c2=O)c1 ; CC(=O)N(C)Cc1cc(C(c2nncn2C)C2CCC2)cc(-n2cc3c(C(F)(F)F)cccn3c2=O)c1 ; CC(=O)N(C)Cc1cc([C@@H](c2nncn2C)C2CCC2)cc(-n2cc3c(C(F)(F)F)cccn3c2=O)c1]

### 1. Synthesis of 512-1

To a solution of 441-3 (1.2 g, 2.370 mmol, 1 equiv) in dioxane (30 mL) was added Pd(OAc)₂ (0.05 g, 0.237 mmol, 0.1 equiv); bis(adamantan-1-yl)(butyl)phosphane (0.17 g, 0.474 mmol, 0.2 equiv) and TMEDA (0.55 g, 4,733 mmol, 2.00 equiv) in a pressure tank. The mixture was purged with nitrogen for 2 min and then was pressurized to 15 atm with CO/H₂=1:1 at 110° C. for overnight. The reaction mixture was cooled to room temperature and concentrated under vacuum. The residue was purified by silica gel column chromatography, eluted with DCM/MeOH (20:1) to afford 512-1 (800 mg, 68.93%) as a yellow solid.

### 2. Synthesis of 512-2

To a stirred solution of 512-1 (780 mg, 1.713 mmol, 1 equiv) and Methylamine hydrochloride (346.90 mg, 5.139 mmol, 3 equiv) in DCE (20 mL) was added TEA (519.92 mg, 5.139 mmol, 3 equiv) at room temperature. The resulting mixture was stirred for h at room temperature. To the above mixture was added STAB (1088.92 mg, 5.139 mmol, 3 equiv) at room temperature. The resulting mixture was stirred for additional overnight at room temperature. The reaction was quenched with saturated NH₄Cl (aq.) at room temperature. The aqueous layer was extracted with DCM (2×50 mL). The resulting mixture was concentrated under vacuum. The residue was purified by Prep-TLC (DCM/MeOH 15:1) to afford 512-2 (580 mg, 64.78%) as a yellow solid,

### 3. Synthesis of 512-3

To a stirred solution of 512-2 (560 mg, 1.190 mmol, 1 equiv) and TEA (361.33 mag, 3.570 mmol, 3 equiv) in THF (10 mL) was added Ac₂O (364.53 mg, 3,570 mmol, 3 equiv) at room temperature. The resulting mixture was stirred for 3 h at room temperature. The resulting mixture was concentrated under vacuum. The residue was purified by Prep-TLC (DCM/MeOH 12:1) to afford 512-3 (450 mg, 71.55%) as a yellow solid.

### 4. Synthesis of 512

The 512-3 (450 mg) was purified by Prep-Chiral-HPLC with the following conditions (Column: CHIRAL ART Cellulose-SB, 2*25 cm, 5 μm; Mobile Phase A: MtBE (0.5% 2M NH3-MeOH), Mobile Phase B: MeOH:DCM=1:1; Flow rate: 20 mL/min; Gradient: 20% B to 20% B in 13 min; Wave Length: 220/254 umm; RT1 (min): 8.23; RT2 (min): 10.39; the first peak is product) to afford 512 (150.7 mg, 32.58%) as a yellow solid.

LC-MS-512 (ES, m/z): [M+H]⁺ 513. H-NMR-512 (400 MHz, DMSO-d6, δ ppm): 1.68-1.91 (m, 5H), 2.02-2.13 (m, 4H), 2.78-2.89 (s, 3H), 3.18-3.25 (m, 1H), 3.43 (s, 3H), 4.24-4.31 (m, 1H), 4.51-4.58 (s, 2H), 6.26-6.30 (m, 1H), 7.04-7.09 (m, 1H), 7.10-7.12 (m, 1H), 7.28-7.31 (m, 1H), 7.54-7.63 (m, 2H), 7.76-7.78 (d, 1H), 8.33 (s, 1H).

### Example 504. Synthesis of Compound 513

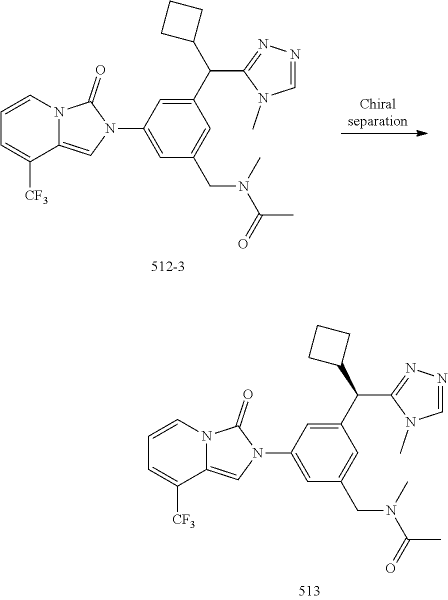 [SMILES: CC(=O)N(C)Cc1cc(C(c2nncn2C)C2CCC2)cc(-n2cc3c(C(F)(F)F)cccn3c2=O)c1 ; CC(=O)N(C)Cc1cc([C@H](c2nncn2C)C2CCC2)cc(-n2cc3c(C(F)(F)F)cccn3c2=O)c1]

### 1. Synthesis of 513

The 512-3 (450 mg) was purified by Prep-Chiral-HPLC with the following conditions (Column: CHIRAL ART Cellulose-SB, 2*25 cm, 5 μm; Mobile Phase A: MtBE (0.5% 2M N43-MeOH), Mobile Phase B: MeOH:DCM=1:1; Flow rate: 20 mL/min; Gradient: 20% B to 20% B in 13 min; Wave Length: 220/254 nm; RT1 (min): 8.23; RT2 (min): 10.39; the second peak is product) to afford 513 (147.3 mag, 32.00%) as a yellow solid,

LC-MS-513 (ES, m/z): [M+H]⁺ 513. H-NMR-513 (400 MHz, DMSO-d6, δ ppm): 1.68-1.91 (m, 5H), 2.02-2.13 (m, 4H), 2.78-2.89 (s, 3H), 3.18-3.25 (m, 1H), 3.43 (s, 3H), 4.24-4.31 (m, 1H), 4.51-4.58 (s, 2H), 6.26-6.30 (m, 1H), 7.04-7.09 (m, 1H), 7.10-7.12 (m, 1H), 7.28-7.31 (m, 1H), 7.54-7.63 (m, 2H), 7.76-7.78 (d, 1H), 8.33 (s, 1H).

### Example 505. Synthesis of Compound 514

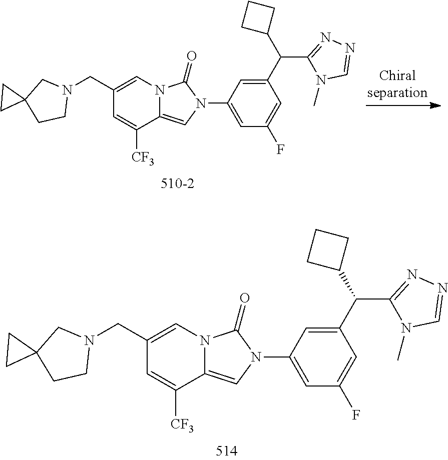 [SMILES: Cn1cnnc1C(c1cc(F)cc(-n2cc3c(C(F)(F)F)cc(CN4CCC5(CC5)C4)cn3c2=O)c1)C1CCC1 ; Cn1cnnc1[C@H](c1cc(F)cc(-n2cc3c(C(F)(F)F)cc(CN4CCC5(CC5)C4)cn3c2=O)c1)C1CCC1]

### 1. Synthesis of 514

The 510-2 (320 mg) was purified by Prep-Chiral-HPLC with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min: Gradient: 30% B to 30% B in 10 min; Wave Length: 220/254 nm; RT1 (min): 7.24; RT2 (min): 8.45; 18 the first peak is product) to afford 514 (112.2 mg, 35.06%) as a yellow solid.

LC-MS-514 (ES, m/z): [M+H]⁺ 555. H-NMR-510 (400 MHz, DMSO-d₆, δ ppm): 0.48-0.58 (d, 4H), 1.68-1.82 (m, 7H), 2.06-2.08 (m, 1H), 2.45-2.50 (m, 2H), 2.63-2.73 (m, 2H), 3.18-3.24 (m, 1H), 3.39 (s, 2H), 3.46 (s, 3H), 4.30-4.33 (d, 1H), 7.04-7.06 (m, 2H), 7.42 (s, 1H), 7.69-7.72 (m, 3H), 8.35 (s, 1H).

### Example 506. Synthesis of Compound 515

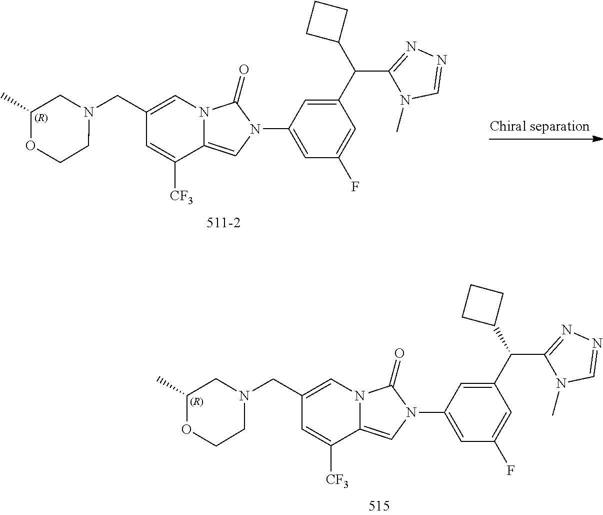

### 1. Synthesis of 515

511-2 (350 ng) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 70% B to 70% B in 10 min; Wave Length: 220/254 nm; RT1 (min): 5.61; RT2 (min): 8.01; the first peak is product) to afford 515 (136.9 mg, 38.25%) as a yellow solid,

LC-MS-515 (ES, m/z): [M+H]⁺ 559. H-NMR-515 (400 MHz, DMSO-d6, δ ppm): 1.03-1.04 (d, 3H), 1.70-1.84 (m, 6H), 2.03-2.12 (m, 2H), 2.65-2.67 (m, 1H), 2.72-2.74 (m, 1H), 3.20-3.27 (m, 1H), 3.27 (s, 2H), 3.45-3.51 (m, 5H), 3.72-3.75 (m, 1H), 4.30-4.33 (d, 1H), 7.03-7.06 (m, 2H), 7.41 (s, 1H), 7.68-7.71 (m, 3H), 8.35 (s, 1H).

### Example 507. Synthesis of Compound 516

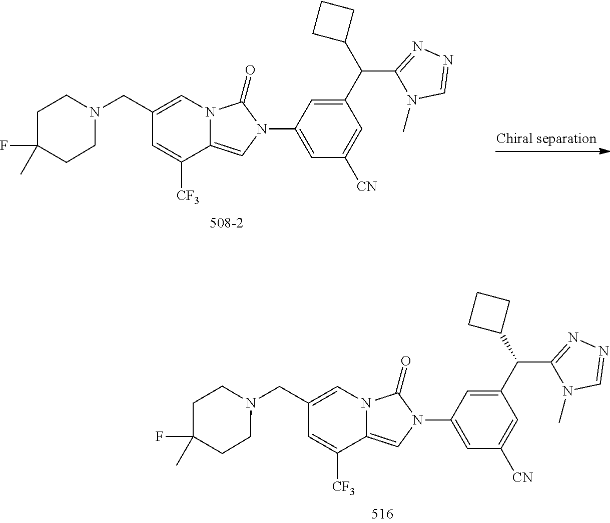 [SMILES: Cn1cnnc1C(c1cc(C#N)cc(-n2cc3c(C(F)(F)F)cc(CN4CCC(C)(F)CC4)cn3c2=O)c1)C1CCC1 ; Cn1cnnc1[C@H](c1cc(C#N)cc(-n2cc3c(C(F)(F)F)cc(CN4CCC(C)(F)CC4)cn3c2=O)c1)C1CCC1]

### 1. Synthesis of 516

The 508-2 (300 mg) was purified by Chiral separation with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 L/min; Gradient: 40% B to 40% B in 10.5 min; Wave Length: 220/254 nm; RT1 (min): 5.87; RT2 (min): 8.06; the second peak is product) to afford 516 (113.8 mg, 37.25%) as a yellow solid.

LC-MS-516 (ES, m/z): [M+H]⁺ 582. H-NMR-516 (400 MHz, DMSO-d6, δ ppm): 1.25-1.39 (d, 3H), 1.52-1.86 (m, 9H), 2.07-2.08 (m, 1H), 2.26-2.32 (m, 2H), 2.62-2.65 (m, 2H), 3.18-3.22 (m, 1H), 3.32 (s, 2H), 3.48 (s, 3H), 4.38-4.40 (d, 1H), 7.04 (s, 1H), 7.52 (s, 1H), 7.68-7.70 (d, 2H), 8.23-8.24 (d, 2H), 8.37 (s, 1H).

### Example 508. Synthesis of Compound 517

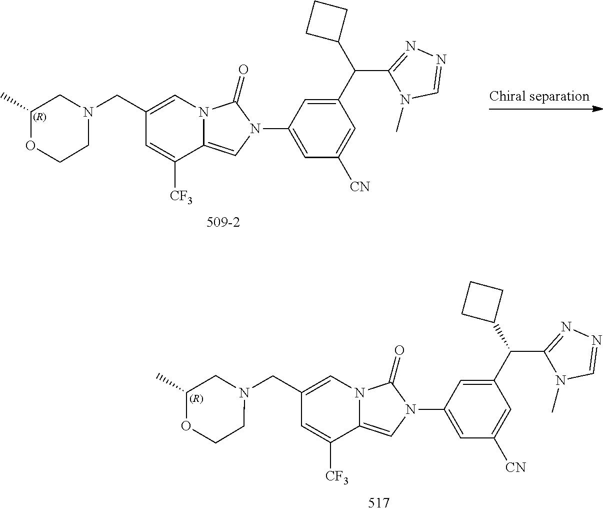

### 1. Synthesis of 517

509-2 (260 mg) was purified by Chiral separation with the following conditions (Column: CHIRALPAK IF, 2*25 cm, 5 prn; Mobile Phase A: Hex (0.5% 2M NH₃-MeOH), Mobile Phase B: EtOH:DCM=1:1; Flow rate: 20 mL/min; Gradient: 40% B to 40% B in 11 min; Wave Length: 220/254 nm; RT1 (min): 6.67; RT2 (min): 8.16; the second peak is product) to afford 517 (96.6 mg, 36.67%) as a yellow solid.

LC-MS-517 (ES, m/z): [M+H]⁺ 566. H-NMR-517 (400 MHz, DMSO-d6, δ ppm): 1.00-1.10 (d, 311), 1.63-1.85 (m, 6H), 2.03-2.11 (m, 2H), 2.61-2.75 (m, 2H), 3.18-3.30 (m, 3H), 3.41-3.51 (m, 5H), 3.73-3.75 (d, 1H), 4.38-4.40 (m, 1H), 7.04 (s, 1H), 7.52 (s, 1H), 7.69-7.70 (d, 2H), 8.23-8.24 (d, 2H), 8.37 (s, 1H).

### Example 509. Synthesis ofCompound 518

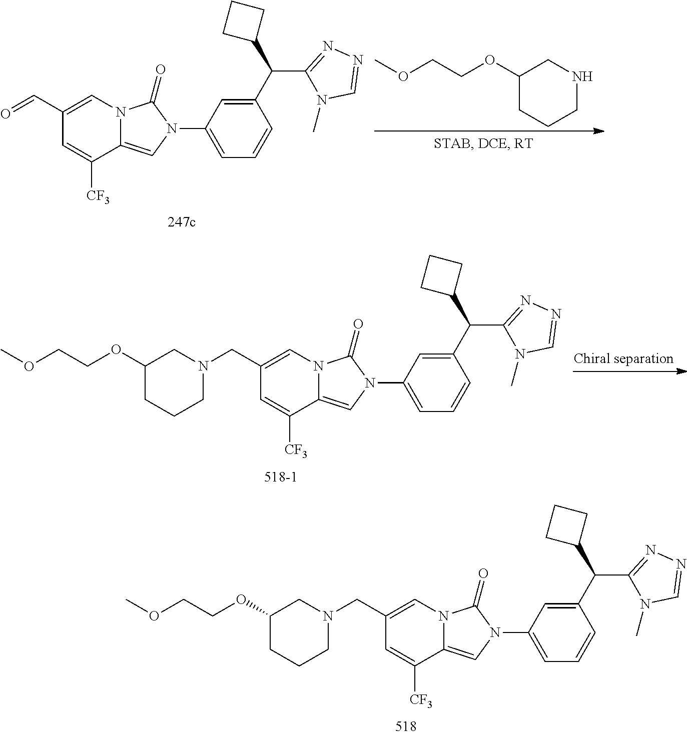

### 1. Synthesis of 518-1

To a stirred solution of 247c (700 mg, 1537 mmol, 1 equiv) in DCE (10 mL) was added 3-(2-methoxyethoxy)piperidine (489.46 rug, 3.074 mmol, 2 equiv) at room temperature. The resulting mixture was stirred for 3 h at room temperature. To the above mixture was added STAB (651.49 mg 3,074 mmol, 2 equiv) at room temperature. The resulting mixture was stirred overnight at room temperature. The reaction was quenched with NH₄Cl (aq.) (80 mL) at room temperature. The aqueous layer was extracted with DCM (2×50 mL). The resulting mixture was concentrated under vacuum. The residue was purified by reverse flash chromatography with the following conditions: column, C18 silica gel; mobile phase, MeCN in Water (10 mmol/L NH4HCO₃), 10% to 70% gradient in 30 min; detector, UV 254 nm. The residue was purified by Prep-TLC (DCM/MeOH 20:1) to afford 518-1 (300 mg, 31.63%) as a yellow solid.

### 2. Synthesis of 518

The 518-1 (300 mg) was purified by Prep-Chiral-HPLC with the following conditions (Column: CHIRALPAK IH, 2*25 cm, 5 μm; Mobile Phase A: Hex (0.5% 2M NH₃-MeOH), Mobile Phase B: EtOH:DCM=1:1; Flow rate: 20 mL/min; Gradient: 20% B to 20% B in 20 min; Wave Length: 220/254 nm; RT1 (min): 6.27; RT2 (min): 11.95; the first peak is product) to afford the crude product. The residue was purified by Prep-TLC (DCM/MeOH 20:1) to afford the 518 (82.2 mg, 26.55%) as a yellow solid, LC-MS-51.8 (ES, m/z): [M+H]⁺599.

H-NMR-518 (400 MHz, DMSO-d6, δ ppm): 1.08-1.23 (m, 1H), 1.37-1.53 (m, 1H), 1.64-1.74 (m, 2H), 1.74-1.88 (m, 4H), 1.88-1.91 (m, 2H), 1.91-2.03 (m, 1H), 2.03-2.10 (m, 1H), 2.63-2.66 (m, 1H), 2.88-2.93 (m, 1H), 3.20-3.30 (m, 4H), 3.32 (s, 2H), 3.33-3.35 (m, 1H), 3.35-3.40 (m, 2H), 3.43 (s, 3H), 3.50-3.53 (m, 2H), 4.25-4.27 (d, 1H), 7.01 (s, 1H), 7.18-7.20 (d, 1H), 7.31 (s, 1H), 7.42-7.46 (m, 1H), 7.67-7.70 (m, 2H), 7.73 (s, 1H), 8.32 (s, 1H).

### Example 510. Synthesis of Compound 519

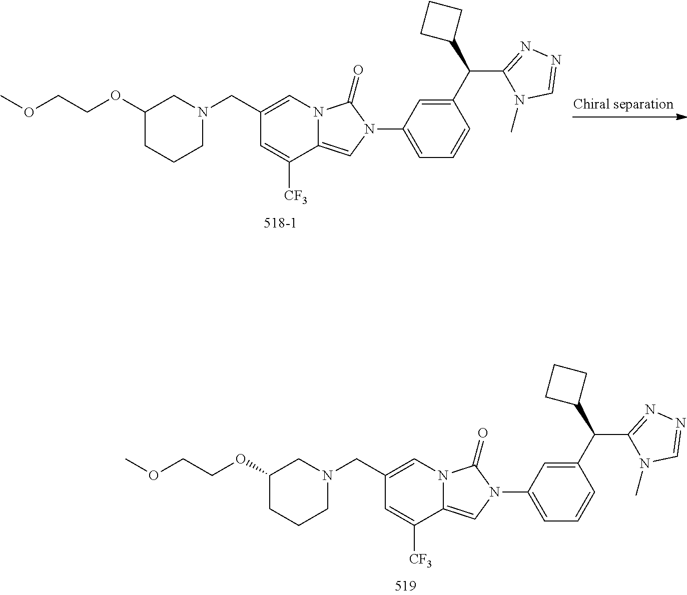 [SMILES: COCCOC1CCCN(Cc2cc(C(F)(F)F)c3cn(-c4cccc([C@H](c5nncn5C)C5CCC5)c4)c(=O)n3c2)C1 ; COCCO[C@H]1CCCN(Cc2cc(C(F)(F)F)c3cn(-c4cccc([C@H](c5nncn5C)C5CCC5)c4)c(=O)n3c2)C1]

### 1. Synthesis of 519

518-1 (300 mg) was purified by Prep-Chiral-HPLC with the following conditions (Column: CHIRALPAK IH, 2*25 cm, 5 um; Mobile Phase A: Hex (0.5% 2M NH₃-MeOH), Mobile Phase B: EtOH:DCM=1:1; Flow rate: 20 mL/min; Gradient: 20% B to 20% B in 20 min; Wave Length: 220/254 nm; RT1 (min): 6.27; RT2 (min): 11.95; the second peak is product) to afford 519 (125.1 mg, 41.49%) as a yellow solid.

LC-MS-519 (ES, m/z): [M+H]⁺ 599. H-NMR-519 (400 MHz, DMSO-d6, δ ppm): 1.08-1.23 (m, 1H), 1.37-1.53 (m, 1H), 1.64-1.74 (m, 2H), 1.74-2.03 (m, 7H), 2.03-2.10 (m, 1H), 2.63-2.66 (m, 1H), 2.88-2.93 (m, 1H), 3.20-3.30 (m, 4H), 3.32 (s, 2H), 3.33-3.35 (m, 1H), 3.35-3.40 (m, 2H), 3.43 (s, 3H), 3.50-3.53 (m, 2H), 4.25-4.27 (d, 1H), 7.01 (s, 1H), 7.18-7.20 (d, 1H), 7.31 (s, 1H), 7.42-7.46 (m, 1H), 7.67-7.70 (m, 2H), 7.73 (s, 1H), 8.32 (s, 1H).

### Example 511. Synthesis of Compound 520

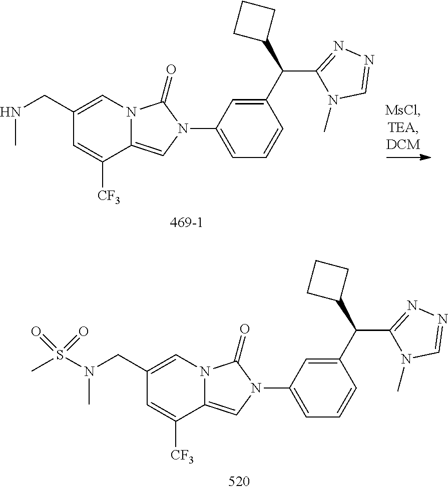 [SMILES: CNCc1cc(C(F)(F)F)c2cn(-c3cccc([C@H](c4nncn4C)C4CCC4)c3)c(=O)n2c1 ; CN(Cc1cc(C(F)(F)F)c2cn(-c3cccc([C@H](c4nncn4C)C4CCC4)c3)c(=O)n2c1)S(C)(=O)=O]

### 1. Synthesis of 520

Into a 40 mL sealed tube were added 469-1 (380 mg, 0.808 mmol, 1 equiv), DCM (5 mL), TEA (245.19 mg, 2.424 mmol, 3 equiv) and MsCl (101.76 mg, 0,889 mmol, 1.1 equiv) at room temperature. The final reaction mixture was irradiated with microwave radiation for 1.5 h at room temperature. The reaction was quenched with sat. NaHCO₃ (aq.) (20 mL) at room temperature. The aqueous layer was extracted with CH₂Cl₂ (3×10 mL). The resulting mixture was concentrated under reduced pressure. The residue was purified by Prep-TLC (CH₂Cl₂/MeOH 15:1). The crude product (180 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 48% B in 8 min; Wave Length: 254 nm; RT1 (min): 7.57) to afford 520 (81.6 mg, 18.25%) as a yellow solid.

LC-MS-520 (ES, m/z): [M+H]⁺ 549. H-NMR-520 (400 MHz, DMSO, δ ppm): 1.65-1.93 (m, 5H), 2.04-2.20 (s, 1H), 2.69-2.80 (s, 3H), 2.94-3.08 (s, 3H), 3.18-3.29 (m, 1H), 3.39-3.50 (s, 3H), 4.02-4.17 (s, 2H), 4.21-4.38 (d, 1H), 6.87-6.99 (s, 1H), 7.16-7.27 (d, 1H), 7.31-7.41 (s, 1H), 7.41-7.55 (m, 1H), 7.62-7.73 (m, 1H), 7.73-7.80 (s, 1H), 7.80-7.87 (s, 1H), 8.26-8.42 (s, 1H).

### Example 512. Synthesis of Compound 521

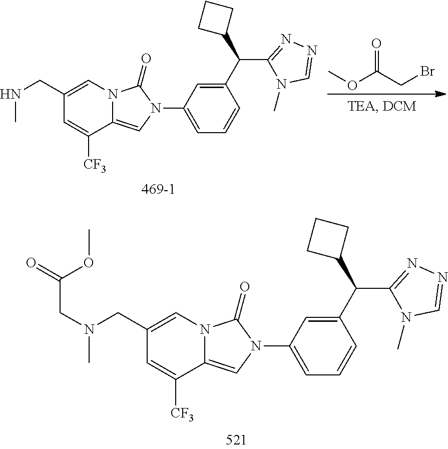

### 1. Synthesis of 521

Into a 20 mL sealed tube were added 469-1 (350 mg, 0.744 mmol, 1 equiv), DCM (4 mL), TEA (225.83 mg, 2,232 mmol, 3 equiv) and methyl 2-bromoacetate (136.56 mg, 0,893 mmol, 1.2 equiv) at room temperature. The final reaction mixture was irradiated with microwave radiation for 1.5 h at room temperature. The reaction was quenched with sat. NaHCO₃ (aq.) (20 mL) at room temperature. The aqueous layer was extracted with CH₂Cl₂ (3×10 mL). The resulting mixture was concentrated under reduced pressure. The residue was purified by Prep-TLC (CH₂Cl₂/MeOH 15:1). The crude product (200 mg) was purified by Prep-HPLC with the following conditions (Column: XBridge Prep OED C18 Column, 30*150 mm, 5 μm; Mobile Phase A: Water (10 mmol/L NH4HCO3), Mobile Phase B: ACN; Flow rate: 60 mL/min; Gradient: 33% B to 51% B in 8 min; Wave Length: 254 nm; RT1 (min): 7.57) to afford 521 (100.3 mg, 24.53%) as a yellow solid.

LC-MS-521 (ES, m/z): [M+H]⁺ 543. H-NMR-521-1724 (400 MHz, DMSO, δ ppm): 1.60-1.91 (m, 5H), 2.01-2.16 (s, 1H), 2.22-2.35 (s, 3H), 3.14-3.30 (m, 1H), 3.36-3.39 (s, 2H), 3.40-3.46 (s, 5H), 3.58-3.71 (s, 3H), 4.18-4.31 (d, 1H), 6.99-7.12 (s, 1H), 7.12-7.29 (d, 1H), 7.29-7.37 (s, 1H), 7.37-7.52 (m, 1H), 7.62-7.74 (m, 2H), 7.74-7.83 (s, 1H), 8.25-8.41 (s, 1H).

### Example 513. Synthesis of Compound 522

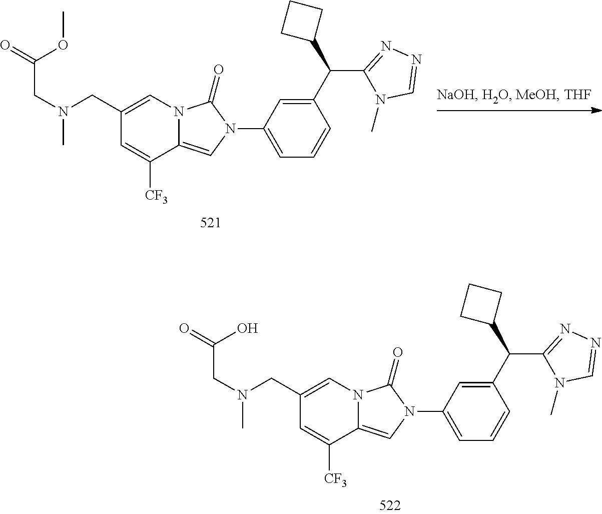 [SMILES: COC(=O)CN(C)Cc1cc(C(F)(F)F)c2cn(-c3cccc([C@H](c4nncn4C)C4CCC4)c3)c(=O)n2c1 ; CN(CC(=O)O)Cc1cc(C(F)(F)F)c2cn(-c3cccc([C@H](c4nncn4C)C4CCC4)c3)c(=O)n2c1]

### 1. Synthesis of 522

Into a 20 mL sealed tube were added 521 (280 mg, 0.516 mml, 1 equiv), MeOH (1 mL), THF (1 mL), 1120 (3 mL) and NaOH (61.92 mg, 1.548 mmol, 3 equiv) at room temperature. The final reaction mixture was irradiated with microwave radiation for 4 h at room temperature. The reaction was quenched with 1 M HC (20 mL) at room temperature. The aqueous layer was extracted with CH2Cl2 (3×10 mL). The resulting mixture was concentrated under reduced pressure. The residue was purified by Prep-TLC (CH₂Cl₂/MeOH 15:1). The crude product (190 mg) was purified by Prep-HPLC with the following conditions (Column: XBridge Prep OBD C18 Column, 30*150 mm, 5 pnm; Mobile Phase A: Water (10 mmol/L NH4HCO3), Mobile Phase B: ACN; Flow rate: 60 mL/min; Gradient: 12% B to 36% B in 8 min; Wave Length: 254 um; RT1 (min): 7.45) to afford 522 (92.1 mg, 33.70%) as a yellow solid.

LC-MS-522 (ES, m/z): [M+H]⁺ 529. H-NMR-522 (400 MHz, DMSO, δ ppm): 1.65-1.90 (m, 5H), 2.00-2.17 (s, 1H), 2.23-2.34 (s, 3H), 3.15-3.30 (s, 3H), 3.43-3.46 (s, 3H), 3.46-3.51 (d, 2H), 4.12-4.43 (d, 1H), 7.06-7.15 (s, 1H), 7.15-7.27 (m, 1H), 7.27-7.40 (s, 1H), 7.40-7.51 (m, 1H), 7.64-7.73 (m, 2H), 7.73-7.86 (s, 1H), 8.28-8.41 (s, 1H).

### Example 514. Synthesis of Compound 523

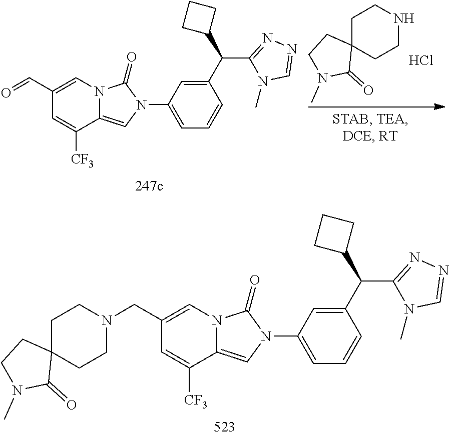

### 1. Synthesis of 523

To a stirred solution of 247c (500 mg, 1.098 mmol, 1.00 equiv) and 2-methyl-2,8-diazaspiro [4.5] decan-1-one hydrochloride (337.09 mg, 1.647 mmol, 1.5 equiv) in DCE (10 mL) were added TEA (222.19 mg, 2.196 mmol, 2 equiv) at room temperature. The resulting mixture was stirred for 1 h at room temperature. To the above mixture was added STAB (465.35 mg, 2.196 mmol, 2 equiv) at room temperature. The resulting mixture was stirred overnight at room temperature. The reaction was quenched with saturated NH₄Cl (aq.) (50 mL) at room temperature. The resulting mixture was extracted with CH₂Cl₂ (3×40 mL). The resulting mixture was concentrated under reduced pressure. The residue was purified by Prep-TLC (CH₂Cl₂/MeOH 10:1) to afford 523 (124.2 mg, 18.39%) as a yellow solid.

LC-MS-523 (ES, m/z): [M+H]⁺ 608. H-NMR-523 (400 MHz, DMSO-d6, δ ppm): 1.25-1.35 (m, 2H), 1.61-1.70 (m, 3H), 1.71-1.78 (m, 4H), 1.79-1.83 (m, 2H), 2.01-2.13 (m, 311), 2.71 (s, 3H), 2.72-2.83 (m, 2H), 3.18-3.31 (m, 5H), 3.43 (s, 3H), 4.25-4.27 (d, 1H), 7.03 (s, 1H), 7.18-7.20 (d, 1H), 7.32 (s, 1H), 7.42-7.46 (m, 1H), 7.66-7.73 (m, 1H), 8.33 (s, 1H).

### Example 515. Synthesis of Compound 524

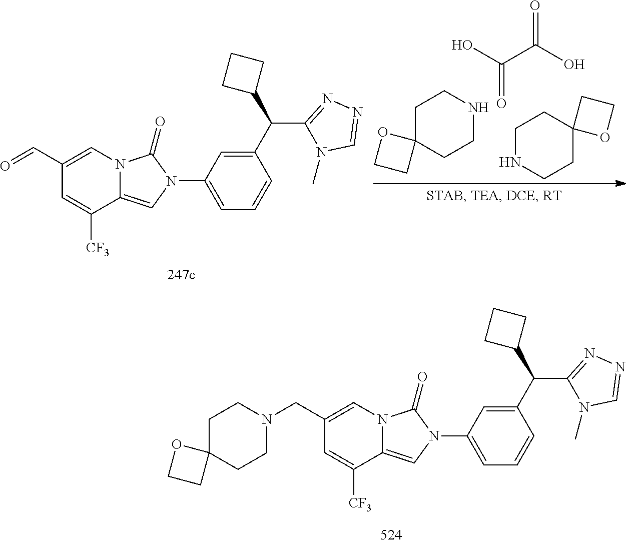

### 1. Synthesis of 524

To a stirred solution of 247c (500 mg, 1.098 mmol, 1.00 equiv) and I-Oxa-7-azaspiro[3.5]nonane hemioxalate (378.10 mg, 1.098 mmol, 1 equiv) in DCE (10 mL) were added TEA (166.64 mg, 1.647 mmol, 1.5 equiv) at room temperature. The resulting mixture was stirred for 1 h at room temperature. To the above mixture was added STAB (465.35 mg, 2.196 mmol, 2 equiv) at room temperature. The resulting mixture was stirred overnight at room temperature. The reaction was quenched with saturated NH₄Cl (aq.) (80 mL) at room temperature. The resulting mixture was extracted with CH₂Cl₂ (3×40 mL). The resulting mixture was concentrated under reduced pressure. The crude product (178 mg) was purified by Prep-HPLC with the following conditions (Column: XBridge Prep OBD C18 Column, 30*150 mm, 5 μm; Mobile Phase A: Water (10 mmol/L NH4HCO₃), Mobile Phase B: ACN; Flow rate: 60 mL/min Gradient: 33% B to 54% B in 8 min, Wave Length: 254 nm; RT1 (min): 7.9) to afford 524 (145.9 mg, 23.01%) as a yellow solid.

LC-MS-524: (ES, m/z): [M+H]⁺ 567

H-NMR-524: (400 MHz, DMSO-d6, δ ppm): δ 1.61-1.85 (m, 9H), 2.07-2.09 (m, 1H), 2.29-2.32 (m, 4H), 2.46-2.51 (m, 2H), 3.19-3.25 (m, 3H), 3.43 (s, 3H), 4.25-4.27 (d, 1H), 4.34-4.38 (m, 2H), 7.00 (s, 1H), 7.18-7.20 (d, 1H), 7.32 (s, 1H), 7.42-7.46 (m, 1H), 7.66 (s, 1H), 7.66-7.70 (d, 1H), 7.73 (s, 1H), 8.33 (s, 1H).

### Example 516. Synthesis of Compound 525

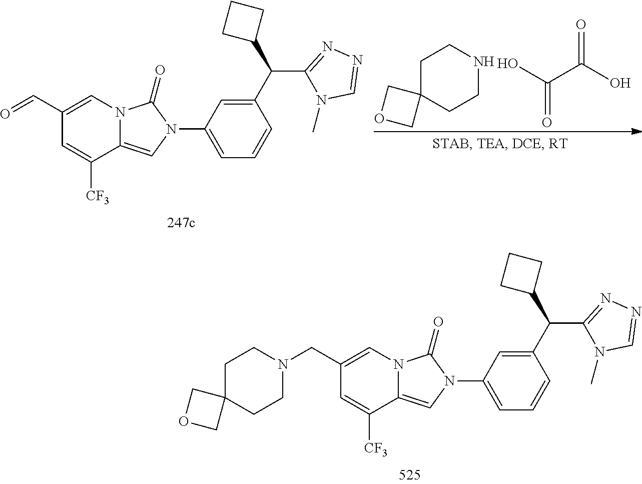

### 1. Synthesis of 525

To a stirred mixture of 247c (500 mg, 1,098 mmol, 1 equiv) and 2-oxa-7-azaspiro[3,5]nonane oxalate (209.45 mg, 1.647 mmol, 1.5 equiv) in DCE (6 mL) was added TEA (333.28 mg, 3.294 mmol, 3 equiv) at room temperature. The resulting mixture was stirred for 30 min at room temperature. To the above mixture was added STAR (698.03 mg, 3.294 mmol, 3 equiv) at room temperature. The resulting mixture was stirred for additional 4 h at room temperature. The reaction was quenched with saturated NaHCO₃ (aq.) (80 mL) at room temperature. The aqueous layer was extracted with CH₂Cl₂ (2×30 mL). The residue was purified by Prep-TLC (CH2Cl2/MeOH=15:1) to afford 525 (213.6 mg, 33.65%) as a yellow solid.

LC-MS-525: (ES, m/z): [M+H]⁺ 567. H-NMR-525: (400 MHz, DMSO-d6, δ ppm): 1.71-1.91 (m, 9H), 1.99-2.08 (m, 2H), 2.08-2.41 (m, 3H), 3.13-3.23 (m, 4H), 3.23 (s, 3H), 4.17-4.37 (m, 5H), 7.00 (s, 1H), 7.19-7.21 (m, 1H), 7.21-7.31 (s, 1H), 7.31-7.38 (m, 1H), 7.38-7.42 (m, 1H), 7.65-7.73 (m, 3H), 8.33 (s, 1H).

### Example 517. Synthesis of Compound 526

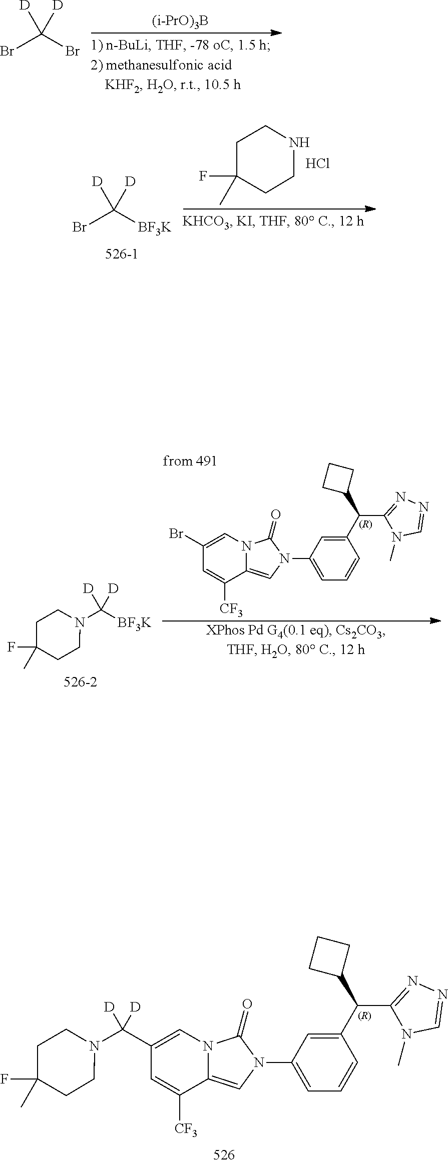

### 1. Synthesis of 526-1

To a solution of dibromo(dideuterio)methane (5 g, 28.43 mmol, 1 equiv) and triisopropyl borate (5.3 g, 28.43 mmol, 6.54 mL, 1 equiv) in THF (39 mL) was added n-BuLi (2.5 M, 11.37 mL, 1 equiv) dropwise at −78° C. Then the reaction was stirred for 1.5 hr. Methanesulfonic acid (1.37 g, 14.22 mmol, 1.01 mL, 0.5 equiv) was added to the mixture at this temperature. After addition, the mixture was stirred at 0° C. for 30 min. Then KHF₂ (6.66 g, 85.30 mmol, 2.81 mL, 3 equiv) and H2O (7.8 mL) was added dropwise to the reaction mixture at 0° C. for 30 min. Then the mixture was stirred at 25° C. for 9.5 hr. The crude product was triturated with acetone (40 mL) at 35° C. for 1 hr to afford 526-1 (5 g, 41%) as a white solid.

### 2. Synthesis of 526-2

To a solution of [bromo(dideuterio)methyl]-trifluoro-boron;potassium hydride (130 mg 640.88 umol, 1 equiv) and 4-fluoro-4-methyl-piperidine (98.45 mg, 640.88 umol, 1 equiv, HCl) in THY (3 mL) was added KHCO₃ (128.32 mg 1: 28 mmol, 2 equiv), 4-fluoro-4-methyl-piperidine (98.45 mg, 640.88 umol, 1 equiv, HCl) and KI (10.64 mg, 64.09 umol, 0.1 equiv). The resulting mixture was stirred at 80° C. for 12 hr. The reaction was concentrated under reduced pressure, then the residue was triturated with acetone (20 mL) at 35° C. for 1 hr. Then the mixture was filtered and the filtrate was concentrated in vacuum to afford 526-2 (0.111 g, 72.4%) as a yellow oil.

### 3. Synthesis of 526

A mixture of 526-2 (225 mg, 941.03 umol, 1 equiv), 6-bromo-2-[3-[(R)-cyclobutyl-(4-methyl-1,2,4-triazol-3-yl)methyl]phenyl]-8-(trifluoromethyl)imidazo[1,5-a]pyridin-3-one (285.88 mg, 564.62 umol, 0.6 equiv), Xphos Pd G4 (80.97 mg, 94.10 umol, 0.1 equiv), Cs2CO₃ (919.82 mg, 2.82 mmol, 3 equiv) in THF (5 mL)/H₂O (1 mL) was degassed and purged with N₂ for 3 times, and then the mixture was stirred at 80° C. for 12 hr under N₂ atmosphere. The reaction was poured into water (10 mL) and the resulting mixture was extracted with EtOAc (10 mL×2). The organic phase was washed with brine (10 mL), dried over anhydrous Na₂SO₄, concentrated in vacuum to give a residue. The residue was purified by normal phase SiO2 chromatography (10-100% MeOH/DCM) to give crude product. The crude product (250 mg) was purified by prep-HPLC with the following conditions (column: Phenomenex Luna C18 200*40 mm*10 um; Mobile phase A: Water (FA), Mobile Phase B: ACN; Flow rate: 50 mL/min; Gradient: 10% B to 40% B in 8 min; Wave Length: 220 nm) to afford 526 (96.8 mg, 37.5%) as a yellow solid.

MS: (ES, m/z): [M+H]⁺ 559.27. H-NMR (400 MHz, METHANOL-d4) δ 8.36 (s, 1H), 7.72-7.68 (m, 1H), 7.68-7.60 (m, 2H), 7.52-7.44 (m, 1H), 7.29-7.23 (m, 1H), 7.13-7.08 (m, 2H), 4.32-4.25 (m, 1H), 3.54 (s, 3H), 2.77-2.74 (m, 2H), 2.52-2.39 (m, 2H), 2.30-2.19 (m, 1H), 2.01-1.63 (m, 10H), 1.40-1.31 (m, 3H).

### Example 518. Synthesis of Compound 527

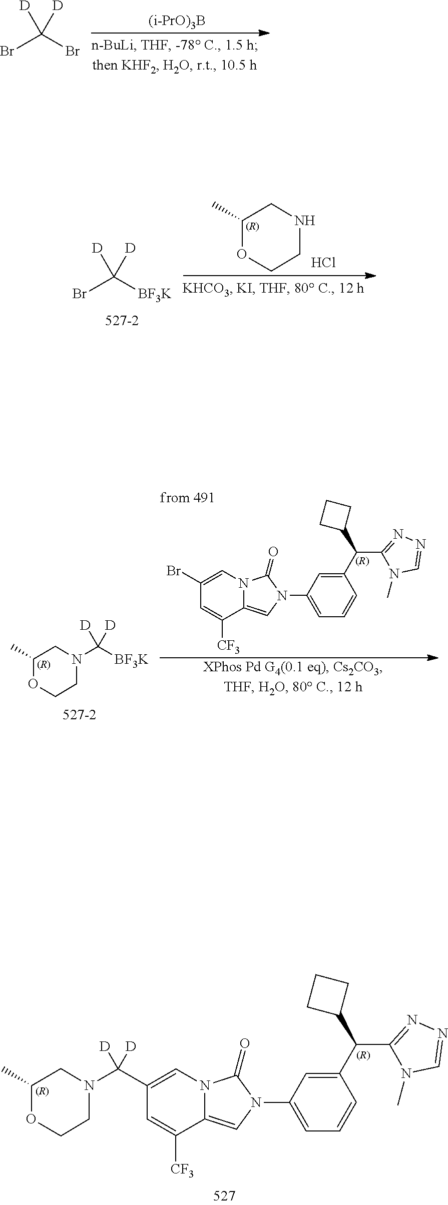

### 1. Synthesis of 527-1

To a solution of dibromo(dideuterio)methane (5.0 g, 28.4 mmol, 1.0 equiv) and triisopropyl borate (5.3 g, 28.4 mmol, 6.5 mL, 1.0 equiv) in TI-if (39.0 mL) was added n-BuLi (2.5 M, 11.4 mL, 1.0 equiv) dropwise at −78° C. Then the reaction was stirred for 1.5 hour, methanesulfonic acid (1.4 g, 14.2 mmol, 1.0 mL, 0.5 equiv) was added to the mixture at this temperature. After addition, the mixture was stirred at 0° C. for 30 minutes. Then KHF₂ (6.7 g, 85.3 mmol, 2.8 mL, 3.0 equiv) and 1-12 (7.8 mL) was added dropwise to the reaction mixture at 0° C. for 30 minutes. Then the mixture was stirred at 25° C. for 9.5 hours. The reaction was concentrated in reduced pressure. The crude product was triturated with acetone (40.0 mL) at 35° C. for 1 hour to afford 527-1 (3.0 g, 52.01% yield) was obtained as a white solid.

### 2. Synthesis of 527-2

To a solution of 527-1 (294 mg, 1.4 mmol, 1.0 equiv) and (2R)-2-methylmorpholine (200 mg, 1.4 mmol, 1.0 equiv, HCl) in THF (5.0 mL) was added KHCO₃ (291 mg, 2.9 mmol, 2.0 equiv) and KI (24 mg, 0.1 equiv). The mixture was stirred at 80° C. for 12 hours. The reaction was concentrated in reduced pressure. The residue was triturated with acetone (5.0 mL) at 35° C. for 1 hour. Then the mixture was filtered and the filtrate was concentrated in vacuum to afford 527-2 (300 mg, 92.53% yield) was obtained as a white solid.

### 3. Synthesis of 527

mixture of 527-2 (165 mg, 1.5 equiv), 6-bromo-2-[3-[(R)-cyclobutyl-(4-methyl-1,2,4-triazol-3-yl)methyl]phenyl]-8-(trifluoromethyl)imidazo[1,5-a]pyridin-3-one (250 mg, 1.0 equiv), CS₂CO₃ (482 mg, 3.0 equiv), dicyclohexyl-[2-(2,4,6-triisopropylphenyl)phenyl]phosphane;methanesulfonate;[2-[2-(methylamino)phenyl]phenyl]palladium(1+) (42 mg, 0.1 equiv) in THF (6.0 mL) and H2O (1.5 mL) was degassed and purged with N₂ for 3 times, and then the mixture was stirred at 80° C. for 12 hours under N atmosphere. The reaction was poured into water (20.0 mL) and the resulting mixture was extracted with EtOAc (2×15.0 mL). The organic phase was washed with brine (10.0 mL), dried over anhydrous Na2SO₄, concentrated in vacuum to give a residue. The residue was purified by prep-HPLC (Column: Phenomenex Luna C18 75*30 mm*3 um; Mobile Phase A: Water (0.2% formic acid), Mobile Phase B: ACN; Flow rate: 25 mL/min; Gradient: 20% B to 60% B in 8 min; Wave Length: 220 nm) to afford 527 (85 mg, 31.36% yield) was obtained as a yellow solid,

MS: (ES, m/z): [M+H]+ 543.3. H-NMR: (400 MHz, METHANOL-d4) δ ppm 8.38 (s, 1H), 7.69 (s, 1H), 7.68-7.62 (m, 2H), 7.54-7.46 (m, 1H), 7.27 (d, J=7.6 Hz, 1H), 7.12 (br d, J=5.2 Hz, 2H), 4.29 (d, J=10.8 Hz, 1H), 3.92-3.82 (m, 1H), 3.71-3.62 (m, 2H), 3.59-3.51 (m, 3H), 3.31-3.23 (m, 1H), 2.88-2.71 (m, 2H), 2.30-2.17 (m, 2H), 1.96-1.85 (m, 5H), 1.82-1.74 (m, 1H), 1.15 (d, J=6.4 Hz, 3H).

### Example 519. Synthesis of Compound 528

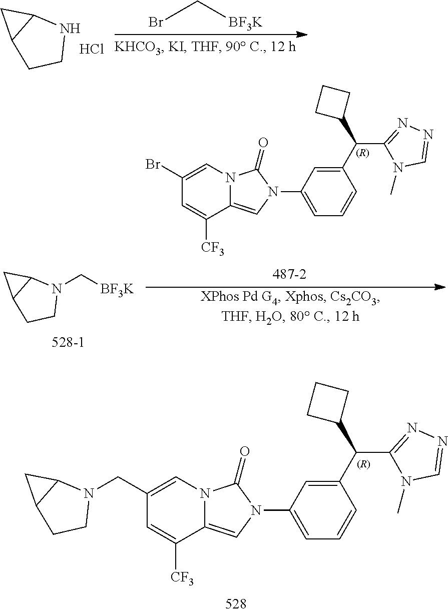

### 1. Synthesis of 528-1

A mixture of 2-azabicyclo[3.1.0]hexane (0.3 g, 2.51 mmol, 1 equiv, HC), potassium;bromomethyl (trifluoro)boranuide (504 mg 2.51 mmol, 1 equiv), KHCO₃ (502.31 mg, 5.02 mmol, 2 equiv) and KI (41.6 mg, 251 umol, 0.1 equiv) in THF (5 mL) was stirred at 90° C. for 12 h under nitrogen atmosphere. The mixture was concentrated under reduced pressure. The crude product was triturated with acetone (50 mL) at 35° C. for 1 h. Then the mixture was filtered and the filtrate was concentrated under reduced pressure to afford 528-1 (410 mg, crude) as a yellow oil.

### 2. Synthesis of 528

To a solution of 487-2 (0.3 g, 592 umol, 1 equiv) and 528-1 (361 mg, 1.78 mmol, 3 equiv) in THF (8 mL) and H2O (2 mL) was added XPhos (28.2 mg, 59.2 umol, 0.1 equiv), dicyclohexyl-2-[(2,4,6-triisopropylphenyl)phenyl]phosphane;methanesulfonate;[2-[2-(methylamino)phenyl]-phenyl]palladium(1+) (50.9 mg, 59.2 umol, 0.1 equiv) and Cs₂CO₃ (579 mg, 1.78 mmol, 3 equiv). The mixture was stirred at 80° C. for 12 h under nitrogen atmosphere. The mixture was extracted with water (50 ml) and EtOAc (30 ml×3). The combined organic layers were dried over anhydrous Na2SO, filtered and the filtrate was concentrated in vacuum. The residue was purified by silica gel column chromatography eluted with DCM/MeOH (10:1). The crude product (230 mg) was purified by Prep-HPLC with the following conditions (column: Phenomenex Luna C18 200*40 mm*10 um; Mobile Phase A: water (FA), Mobile Phase B: ACN; Flow rate: 50 mL/min; Gradient: 1% B to 35% B in 8 min; Wave Length: 220 nm; RT1 (min): 6.2) to afford 528 (32 mg, 10%) as a yellow solid.

MS-528: (ES, m/z): [M+H]⁺ 523.2.

¹H-NMR-528: (400 MHz, METHANOL-d4) δ 8.44-8.40 (m, 1H), 8.36 (s, 1H), 7.76 (s, 1H), 7.66-7.60 (m, 2H), 7.48 (t, J=7.6 Hz, 1H), 7.26 (d, J=7.6 Hz, 1H), 7.13 (d, J=6.0 Hz, 2H), 4.28 (d, J=10.8 Hz, 1H), 3.66-3.56 (m, 2H), 3.54 (s, 3H), 3.29-3.21 (m, 1H), 2.98 (J=9.2 Hz, 1H), 2.84-2.77 (m, 1H), 2.29-2.15 (m, 2H), 2.11-2.00 (m, 1H), 1.99-1.85 (m, 5H), 1.81-1.70 (m, 1H), 1.60-1.51 (m, 1H), 0.91-0.83 (m, 1H), 0.36-0.28 (m, 1H).

### Example 520. Synthesis of Compound 529

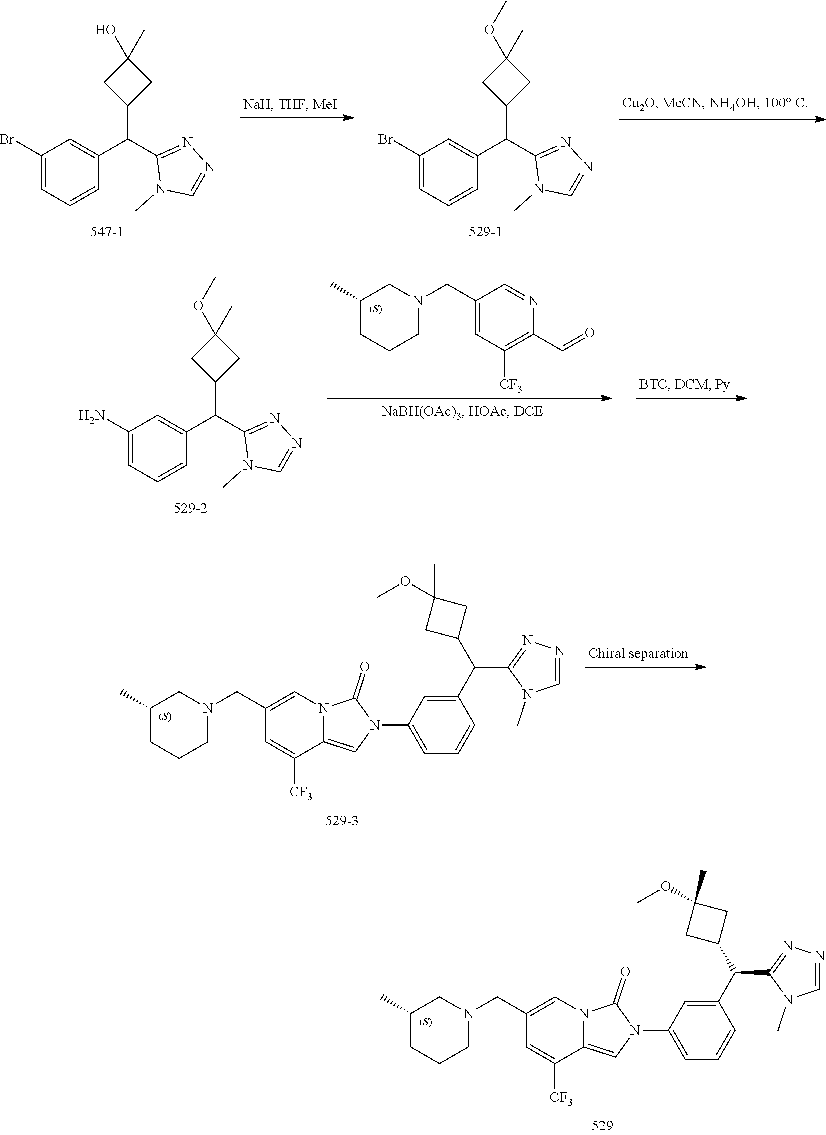

### 1. Synthesis of 529-1

A solution of 574-1 (1.11 g, 3.301 mmol, 1 equiv) in THF (1 mL) was treated with NaH (0.24 g, 5.942 mmol, 1.8 equiv, 60%) for 1 h at 0° C. under nitrogen atmosphere followed by the addition of MeI (1.08 g, 7,592 mmol, 2.3 equiv) at 0° C. The resulting mixture was stirred for 2 h at 0° C. under nitrogen atmosphere. The resulting mixture was diluted with 30 mL of sat.NH₄Cl(aq.). The resulting mixture was extracted with CH₂Cl₂/MeOH (10:1) (3×20 mL). The combined organic layers was concentrated under reduced pressure. The residue was purified by Prep-TLC (CH₂Cl₂/MeOH 20:1) to afford 529-1 (1.22 g, 94.96%) as a yellow solid,

### 2. Synthesis of 529-2

Into a 50 mL pressure tank reactor were added 529-1 (1.22 g, 3.483 mmol, 1 equiv)), Cu20 (0.35 g, 2.438 mmol, 0.7 equiv), MeCN (18 ml) and NH₄OH (18 mL) at room temperature. The mixture was stirred for overnight at 105° C. under nitrogen atmosphere. The resulting mixture was diluted with 100 mL of water. The resulting mixture was extracted with CH₂Cl₂/MeOH (10:1) (3×50 mL). The combined organic layers were concentrated under reduced pressure. The residue was purified by Prep-TLC (CH₂Cl₂/MeOH 25:1) to afford 529-2 (400 mg 38.10%) as a yellow solid.

### 3. Synthesis of 529-3

A solution of 529-2 (400 rug, 1.397 mmol, 1 equiv) in DCE (4 mL) was treated with I-2 (519.85 mg, 1.816 mmol, 1.3 equiv) for 2 h at room temperature under nitrogen atmosphere followed by the addition of NaBH(OAc)3 (444.04 mg, 2.095 mmol, 1.5 equiv) at room temperature. The resulting mixture was stirred for overnight at room temperature under nitrogen atmosphere. The reaction was quenched with sat. NaHCO₃ (aq.) at room temperature. The resulting mixture was extracted with CH₂Cl₂/MeOH (10:1) (3×2 mL). The combined organic layers were concentrated under reduced pressure. The residue was purified by Prep-TLC (CH2Cl2/MeOH 20:1) to afford 529-3 (470 mg, 57.42%) as a yellow solid.

### 4. Synthesis of 5294

To a stirred solution of 529-3 (460 mg, 0.826 mmol, 1 equiv) and Pyridine (392.18 mg, 4.956 mmol, 6 equiv) in DCM (10 mL) was added Triphosgene (98.08 mg, 0.330 mmol, 0.4 equiv) at 0° C. The resulting mixture was stirred for 1 h at 0° C. The reaction was quenched by the addition of sat, NaHCO₃ (aq.) (15 mL) at room temperature. The resulting mixture was extracted with CH₂Cl₂/MeOH=10/1 (2×20 mL). The combined organic layers were concentrated under reduced pressure. The residue was purified by Prep-TLC (CH₂Cl₂/MeOH 15:1) to afford 529-4 (270 mg, 56.08%) as a yellow solid,

### 5. Synthesis of 529

529-4 (270 mg, 0.463 mmol, 1 equiv) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 ML/min; Gradient: 65% B to 65% B in 27 min; Wave Length: 220/254 nm; RT1 (min): 5.42; RT2 (min): 25.02; Sample Solvent: EtOH:DCM=1:1; the first peak was product) to afford −529 (113.4 mg, 42.00%) as a yellow solid.

LCMS-529: (ES, m/z): [M+H]⁺ 583

NMR-529: (400 MHz, CD₃OD, δ ppm): 0.91-0.96 (m, 4H), 1.37 (s, 3H), 1.60-1.78 (m, 5H), 1.81-1.90 (m, 2H), 1.96-2.01 (m, 2H), 2.23-2.28 (m, 1H), 2.89-2.96 (m, 3H), 3.17 (s, 3H), 3.38 (s, 2H), 3.54 (s, 3H), 4.24-4.26 (d, 1H), 7.13-7.14 (d, 2H), 7.27-7.29 (d, 1H), 7.50-7.54 (t, 1H), 7.66-7.70 (m, 3H), 8.38 (s, 1H).

### Example 521. Synthesis of Compound 530

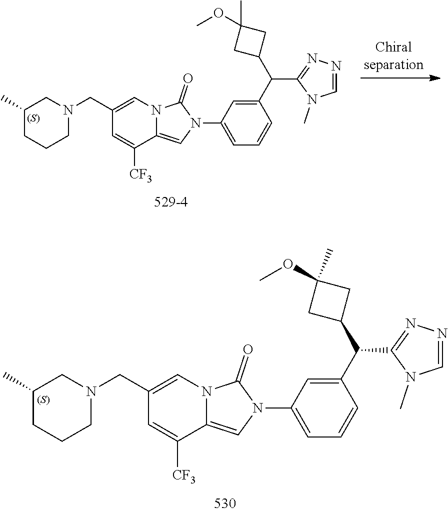

### 1. Synthesis of 530

529-4 (270 mg, 0,463 mmol, 1 equiv) was purified by Prep-HPLC with the following conditions (Column: CHIRALPAK IC, 2*25 cm, 5 μm; Mobile Phase A: Hex (0.5% 2M NH₃-MeOH), Mobile Phase B: EtON: DCM=1:1; Flow rate: 20 mL/min; Gradient: 65% B to 65% B in 27 min; Wave Length: 220/254 nm; RT1 (min): 5.42; RT2 (Min): 25.02; Sample Solvent: EtOH:DCM=1:1; the second peak was product) to afford 530 (91.1 mg, 33.74%) as a yellow solid.

LCMS-530: (ES, m/z): [M+H]⁺ 583

NMR-530: (400 MHz, CD₃OD, δ ppm): 0.91-0.97 (m, 4H), 1.36 (s, 3H), 1.61-1.78 (m, 5H), 1.81-1.89 (m, 2H), 1.96-2.03 (m, 2H), 2.23-2.28 (m, 1H), 2.89-2.96 (m, 3H), 3.17 (s, 3H), 3.38 (s, 2H), 3.54 (s, 3H), 4.24-4.26 (d, 1H), 7.13-7.14 (d, 2H), 7.27-7.29 (d, 1H), 7.50-7.54 (t, 1H), 7.66-7.70 (m, 3H), 8.38 (s, 1H).

### Example 522. Synthesis of Compound 531

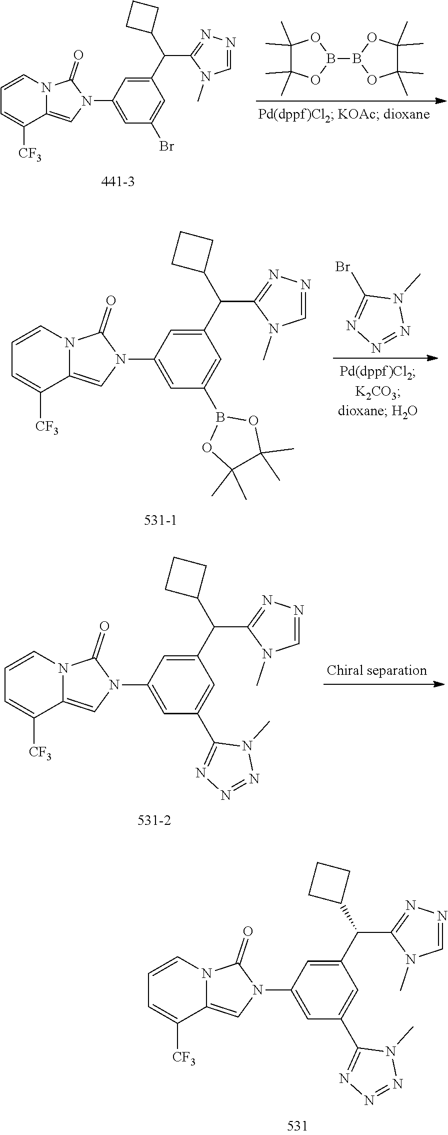

### 1. Synthesis of 531-1

To a stirred mixture of 441-3 (1 g, 1.975 mmol, 1 equiv) and bis(pinacolato)diboron (0.50 g, 1.975 mmol, 1 equiv) indioxane (10 mL) was added KOAc (0.23 g, 2.370 mmol, 1.2 equiv) and Pd(dppf)Cl2 (0.29 g, 0.395 mmol, 0.2 equiv) under nitrogen atmosphere. The resulting mixture was stirred for overnight at 80° C. under nitrogen atmosphere. The resulting mixture was used in the next step directly without further purification.

### 2. Synthesis of 531-2

To a stirred mixture of 531-1 (1.09 g, 1.970 mmol, 1 equiv) and 5-bromo-1-methyl-1,2,3,4-tetrazole (0.48 g, 2.955 mmol, 1.5 equiv) indioxane (10 mL) and H₂O (2 mL) was added K₂CO₃ (0.54 g, 3,940 mmol, 2 equiv) and Pd(dppf)Cl₂ (0.14 g, 0.197 mmol, 0.1 equiv) under nitrogen atmosphere. The resulting mixture was stirred for overnight at 80° C. under nitrogen atmosphere. The resulting mixture was diluted with water (40 mL). The resulting mixture was extracted with EtOAc (3×40 mL). The combined organic layers were concentrated under reduced pressure. The residue was purified by Prep-TLC (CH₂Cl₂/MeOH 20:1) to afford 531-2 (450 mg, 44.84%) as a yellow solid.

### 3. Synthesis of 531

531-2 (450 mg) was purified by Prep-Chiral-HPLC with the following conditions (Column: CHIRALPAK IF, 2*25 cm, 5 μm; Mobile Phase A: MtBE (0.5% 2M NH3-MeOH), Mobile Phase B: MeOH:DCM=1:1; Flow rate: 20 mL/min; Gradient: 25% B to 25% B in 10 min; Wave Length: 220/254 nm; RT1 (min): 7.03; RT2 (min): 9.47; the first peak was product) to afford 531 (197.2 mg, 43.82%) as a yellow solid,

LCMS-531: (ES, m/z): [M+H]⁺ 510

NMR-531: (400 MHz, DMSO, ppm): δ 1.74-1.84 (m, 5H), 2.08-2.12 (m, 1H), 3.26-3.30 (m, 1H), 3.51 (s, 3H), 4.19 (s, 3H), 4.43-4.46 (d, 1H), 6.29-6.33 (t, 1H), 7.12-7.14 (d, 1H), 7.48 (s, 1H), 7.74 (s, 1H), 7.81-7.82 (d, 1H), 8.06 (s, 1H), 8.23 (s, 1H), 8.37 (s, 1H).

### Example 523. Synthesis of Compound 532

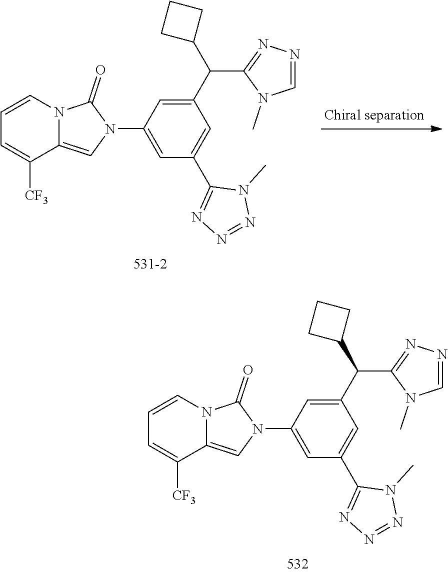 [SMILES: Cn1cnnc1C(c1cc(-c2nnnn2C)cc(-n2cc3c(C(F)(F)F)cccn3c2=O)c1)C1CCC1 ; Cn1cnnc1[C@@H](c1cc(-c2nnnn2C)cc(-n2cc3c(C(F)(F)F)cccn3c2=O)c1)C1CCC1]

### 1. Synthesis of 532-0

531-2 (450 mg) was purified by Prep-HPLC with the following conditions (Column: CHIRALPAK IF, 2*25 cm, 5 μm; Mobile Phase A: MtBE (0.5% 2M NH3-MeOH), Mobile Phase B: MeOH:DCM=1:1; Flow rate: 20 m/min; Gradient: 25% B to 25% B in 10 mi; Wave Length: 220/254 in; RT1 (min): 7.03; RT2 (min): 9.47; the second peak was product) to afford 532 (190.5 mg, 42.33%) as a yellow solid.

LCMS-532: (ES, m/z): [M+H]⁺ 510

NMR-532: (400 MHz, DMSO, ppm): δ 1.72-1.84 (m, 5H), 2.08-2.13 (m, 1H), 3.26-3.28 (m, 1H), 3.51 (s, 3H), 4.19 (s, 3H), 4.43-4.46 (d, 1H), 6.29-6.33 (t, 1H), 7.12-7.14 (d, 1H), 7.48 (s, 1H), 7.74 (s, 1H), 7.81-7.82 (d, 1H), 8.06 (s, 1H), 8.22 (s, 1H), 8.37 (s, 1H).

### Example 524. Synthesis of Compound 533

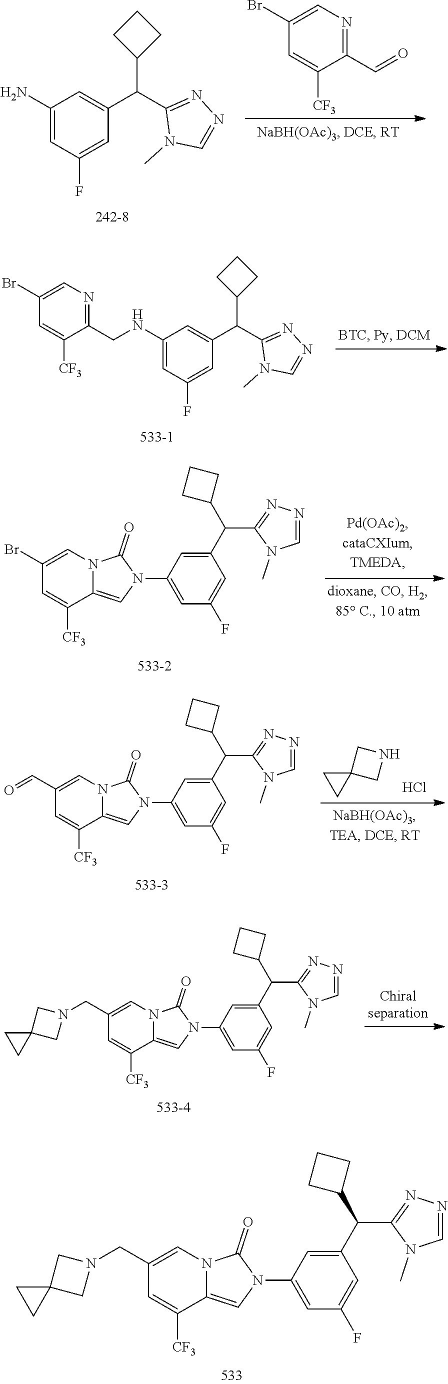

### 1. Synthesis of 533-1

To a stirred solution of 242-8 (1 g, 3.841 mmol, 1 equiv) in DCE (30 mL) was added 5-bromo-3-(trifluoromethyl)picolinaldehyde (1.17 g, 4,609 mmol, 1.2 equiv) at room temperature. The resulting mixture was stirred overnight at room temperature. To the above mixture was added NaBH(OAc)₃ (1.63 g, 7.682 mmol, 2 equiv) at room temperature. The resulting mixture was stirred for additional 4 h at room temperature. The reaction was quenched with saturated NaHCO₃ (aq.) (100 mL) at room temperature. The aqueous layer was extracted with DCM (3×100 mL). The resulting mixture was concentrated under vacuum. The residue was purified by Prep-TLC (DCM/MeOH 20:1) to afford 533-1 (1.1 g, 56.31%) as a white solid.

### 2. Synthesis of 533-2

To a stirred solution of 533-1 (1.1 g, 2.207 mmol, 1 equiv) and Pyridine (1:75 g, 22.070 mmol, 10 equiv) in DCM (30 mL) was added Triphosgene (0.26 g, 0.883 mmol, 0.4 equiv) at 0° C. The resulting mixture was stirred for 10 min at room temperature. The reaction was quenched with saturated NaHCO₃ (aq.) (100 mL) at room temperature. The aqueous layer was extracted with DCM (2×50 mL). The resulting mixture was concentrated under vacuum. The residue was purified by Prep-TLC (DCM/MeOH 20:1) to afford 533-2 (950 mg 77.98%) as an orange solid.

### 3. Synthesis of 533-3

To a solution of 533-2 (900 mg, 1.717 mmol, 1 equiv) and TMEDA (398.95 mg, 3.433 mmol, 2.00 equiv) in 1,4-dioxane (30 mL) were added bis(adamantan-1-yl)(butyl)phosphane (123.09 mg, 0.343 mmol, 0.2 equiv) and Pd(OAc)2 (38.54 mg, 0.172 mmol, 0.1 equiv) in a pressure tank. The mixture was purged with nitrogen for 3 min and then was pressurized to 15 atm with CO/H₂?=1:1 at 85° C. for overnight. The reaction mixture was cooled to room temperature and concentrated under vacuum. The residue was purified by silica gel column chromatography, eluted with DCM/MeOH (20:1) to afford 533-3 (650 mg, 74.39%) as a yellow solid.

### 4. Synthesis of 533-4

To a stirred solution of 533-3 (630 mg, 1.331 mmol, 1 equiv) and 5-azaspiro[2.3]hexane hydrochloride (318.28 mg, 2.662 mmol, 2 equiv) in DCE (8 mL) were added TEA (269.32 mg, 2.662 mmol, 2 equiv) at room temperature. The resulting mixture was stirred for 2 h at room temperature. To the above mixture was added NaBH(OAc)₃ (564.06 mg, 2.662 mmol, 2 equiv) at room temperature. The resulting mixture was stirred for additional 3 h at room temperature. The reaction was quenched with saturated NH₄Cl (aq.) (100 mL) at room temperature. The aqueous layer was extracted with DCM (2×30 mL). The resulting mixture was concentrated under vacuum. The residue was purified by Prep-TLC (DCM/MeOH 15:1) to afford 533-4 (320 mg, 43.60%) as a yellow solid.

### 5. Synthesis of 533

The 533-4 (320 mg) was purified by Prep-Chiral-HPLC with the following conditions (Column: CHIRAL ART Amylose-SA, 2*25 cm, 5 μm; Mobile Phase A: Hex (0.5% 2M NH₃-MeOH), Mobile Phase B: EtOH:DCM=1:1; Flow rate: 20 mL/min; Gradient: 20% B to 20% B in 13.5 min; Wave Length: 220/254 nm; RT1 (min): 6.67; RT2 (min): 9.59; the second peak is product) to afford 533 (122.4 mg, 38.10%) as a yellow solid.

LC-MS-533: (ES, m/z): [M+H]⁺ 541

H-NMR-533: (400 MHz, DMSO-d6, δ ppm): 0.50 (s, 411), 1.67-1.82 (m, 5H), 2.04-2.08 (m, 1H), 3.18-3.24 (m, 1H), 3.24-29 (m, 4H), 3.46 (s, 5H), 4.30-4.33 (d, 1H), 7.02-7.05 (m, 2H), 7.41 (s, 1H), 7.68-7.71 (m, 3H), 8.35 (s, 1H).

### Example 525. Synthesis of Compound 534

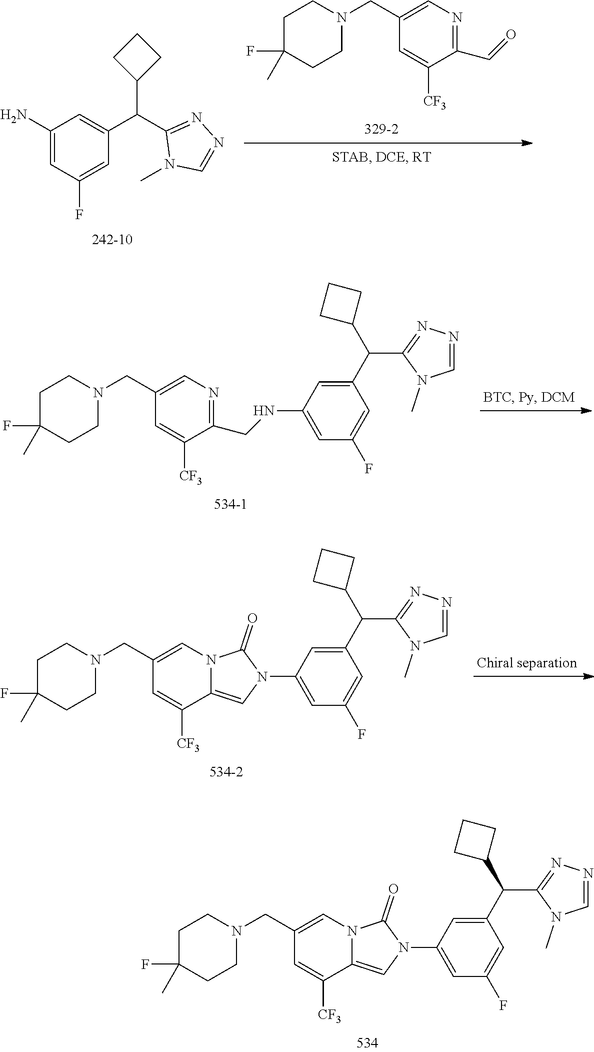 [SMILES: Cn1cnnc1C(c1cc(N)cc(F)c1)C1CCC1 ; CC1(F)CCN(Cc2cnc(C=O)c(C(F)(F)F)c2)CC1 ; Cn1cnnc1C(c1cc(F)cc(NCc2ncc(CN3CCC(C)(F)CC3)cc2C(F)(F)F)c1)C1CCC1 ; Cn1cnnc1C(c1cc(F)cc(-n2cc3c(C(F)(F)F)cc(CN4CCC(C)(F)CC4)cn3c2=O)c1)C1CCC1 ; Cn1cnnc1[C@@H](c1cc(F)cc(-n2cc3c(C(F)(F)F)cc(CN4CCC(C)(F)CC4)cn3c2=O)c1)C1CCC1]

### 1. Synthesis of 534-1

A mixture of 329-2 (600 mg, 1,972 mmol, 1.2 equiv) and 242-10 (427.74 mg, 1.643 mmol, 1 equiv) in DCE (20 mL) was stirred overnight at room temperature. To the above mixture was added STAB (1044.76 mg, 4.930 mmol, 3 equiv) at room temperature. The resulting mixture was stirred for additional 4 h at room temperature. The reaction was quenched with saturated NaHCO₃ (aq.) (80 mL) at room temperature. The aqueous layer was extracted with CH₂Cl₂ (3×40 mL). The resulting mixture was concentrated under vacuum. The residue was purified by Prep-TLC (CH2Cl₂/MeOH 15:1) to afford 534-1 (600 mg, 66.56%) as a white solid.

### 2. Synthesis of 534-2

To a stirred mixture of 534-1 (600 mg, 1.094 mmol, 1 equiv) and Pyridine (519.06 mg, 6.564 mmol, 6 equiv) in DCE (15 mL) was added Triphosgene (126.57 mg, 0.427 mmol, 0.39 equiv) at room temperature. The resulting mixture was stirred for 30 min at room temperature. The reaction was quenched with saturated NaHCO₃ (aq.) (80 mL) at room temperature. The aqueous layer was extracted with CH2Cl₂ (3×40 mL). The resulting mixture was concentrated under vacuum. The residue was purified by Prep-TLC (CH₂Cl₂/MeOH 20:1) to afford 534-2 (320 mg, 50.92%) as a yellow solid.

### 3. Synthesis of 534

The 534-2 (300 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 60% B to 60% B in 10.5 min; Wave Length: 220/254 nm; RT1 (min): 6.43; RT2 (min): 8.34; the second peak is product) to afford 534 (117.5 mg, 39.17%) as a yellow solid.

LC-MS: (ES, m/z): [M+H]⁺ 558. H-NMR: (400 MHz, DMSO-d₆, δ ppm): 1.71-1.74 (m, 3H), 1.60-1.82 (m, 9H), 2.02-2.08 (m, 1H), 2.24-2.33 (m, 2H), 2.51-2.62 (m, 2H), 3.15-3.22 (m, 1H), 3.22 (s, 2H), 3.46 (s, 3H), 4.30-4.33 (d, 1H), 7.03-7.06 (m, 2H), 7.41 (s, 1H), 7.68-7.71 (m, 3H), 8.35 (s, 1H).

### Example 526. Synthesis of Compound 535

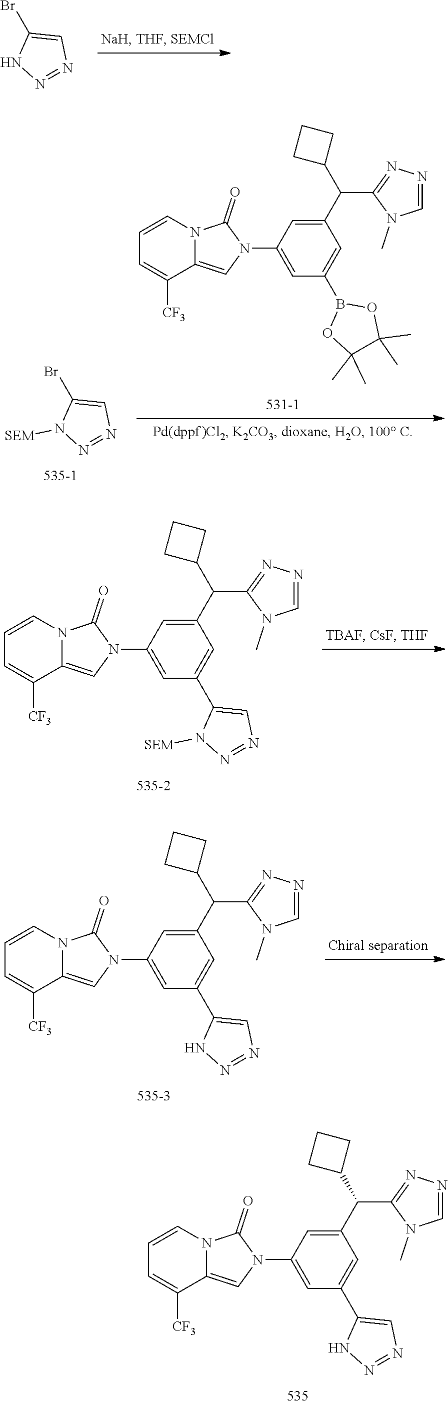 [SMILES: Brc1cnn[nH]1 ; C[Si](C)(C)CCOCn1nncc1Br ; Cn1cnnc1C(c1cc(B2OC(C)(C)C(C)(C)O2)cc(-n2cc3c(C(F)(F)F)cccn3c2=O)c1)C1CCC1 ; Cn1cnnc1C(c1cc(-c2cnnn2COCC[Si](C)(C)C)cc(-n2cc3c(C(F)(F)F)cccn3c2=O)c1)C1CCC1 ; Cn1cnnc1C(c1cc(-c2cnn[nH]2)cc(-n2cc3c(C(F)(F)F)cccn3c2=O)c1)C1CCC1 ; Cn1cnnc1[C@H](c1cc(-c2cnn[nH]2)cc(-n2cc3c(C(F)(F)F)cccn3c2=O)c1)C1CCC1]

### 1. Synthesis of 535-1

To a stirred solution of 4-bromo-3H-1,2,3-triazole (1 g, 6.758 mmol, 1 equiv) in THF (20 mL) was added NaH (0.32 g, 8.110 mmol, 1.2 equiv, 60%) in three portions at 0° C. under nitrogen atmosphere. The resulting mixture was stirred for 30 min at 0° C. under nitrogen atmosphere. To the above mixture was added SEMCl (1.35 g, 8.110 mmol, 1.2 equiv) at 0° C. The resulting mixture was stirred for additional overnight at room temperature. The reaction was quenched by the addition of sat. NH4Cl (aq.) (60 mL). The resulting mixture was extracted with EtOAc (2×30 mL). The combined organic layers were concentrated under reduced pressure. The residue was purified by reverse flash chromatography with the following conditions: column, C18 silica gel; mobile phase, MeCN in Water (10 mmol/L NH4HCO3), 115% to 70% gradient in 30 min; detector, UV 254 nm. This resulted in 535-1 (1.1 g, 58.50%) as a colorless oil.

### 2. Synthesis of 535-2

To a stirred solution of 535-1 (1.01 g, 3.614 mmol, 2 equiv) and 531-1 (1 g, 1.807 mmol, 1 0.00 equiv) indioxane (20 mL) and H₂O (4 mL) were added K₂CO₃ (0.50 g, 3,614 r-mol, 2 equiv) and Pd(dppf)Cl₂ (0.13 g, 0.181 mmol, 0.1 equiv) at room temperature under nitrogen atmosphere. The resulting mixture was stirred for overnight at 100° C. under nitrogen atmosphere. The resulting mixture was diluted with water (60 mL). The resulting mixture was extracted with EtOAc (3×50 mL). The combined organic layers were concentrated under reduced pressure. The residue was purified by silica gel column chromatography, eluted with CH₂Cl₂/MeOH (10:1) to afford 535-2 (950 mg, 84.15%) as a yellow solid.

### 3. Synthesis of 535-3

To a stirred solution of 535-2 (890 mg, 1.425 mmol, 1 equiv) in THF (10 mL) was added TBAF (2.85 mL, 2.850 mmol, 2 equiv, 1M) and CsF (432.80 mg, 2.850 mmol, 2 equiv). The resulting mixture was stirred for overnight at room temperature. The resulting mixture was diluted with EtOAc (30 mL). The resulting mixture was washed with 3×30 mL of water. The resulting mixture was concentrated under reduced pressure. The residue was purified by reverse flash chromatography with the following conditions: column, C18 silica gel; mobile phase, MeCN in Water (10 mmol/L NH₄HCO₃), 5% to 75% gradient in 30 min; detector, UV 254 nm. This resulted in 535-3 as a yellow solid.

### 4. Synthesis of 535

535-3 (410 mg) was purified by Prep-Chiral-HPLC with the following conditions (Column: CHIRALPAK ID, 2*25 cm, 5 μm; Mobile Phase A: MeOH (0.5% 2M NH₃-MeOH), Mobile Phase B: MeOH:DCM=1:1; Flow rate: 20 mL/min; Gradient: 20% B to 20% B in 15 min; Wave Length: 220/254 nm; RT1 (min): 7.65; RT2 (min): 11.85; the first peak was product) to afford 535 (141.2 mg, 34.44%) as a yellow solid.

LCMS: (ES, m/z): [M+H]⁺ 495. NMR: (400 MHz, DMSO, ppm): δ 1.71-1.83 (m, 5H), 2.08-2.13 (m, 1H), 3.21-3.27 (m, 1H), 3.48 (s, 3H), 4.33-4.36 (d, 1H), 6.29-6.32 (t, 1H), 7.11-7.12 (d, 1H), 7.43 (s, 1H), 7.73 (s, 1H), 7.79-7.81 (m, 2H), 8.12 (s, 1H), 8.31-8.64 (m, 2H), 15.09 (d, 1H).

### Example 527. Synthesis of Compound 536

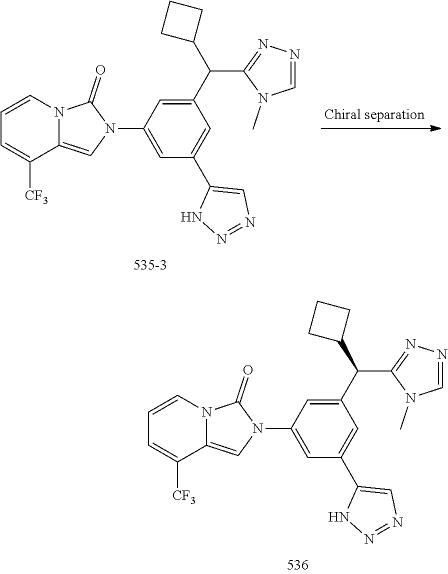 [SMILES: Cn1cnnc1C(c1cc(-c2cnn[nH]2)cc(-n2cc3c(C(F)(F)F)cccn3c2=O)c1)C1CCC1 ; Cn1cnnc1[C@@H](c1cc(-c2cnn[nH]2)cc(-n2cc3c(C(F)(F)F)cccn3c2=O)c1)C1CCC1]

### 1. Synthesis of 536

535-3 (410 mg) was purified by Prep-Chiral-HPLC with the following conditions (Column: CHIRALPAK ID, 2*25 cm, 5 um; Mobile Phase A: MeOH (0.5% 2M NH₃-MeOH), Mobile Phase B: MeOH:DCM=1:1; Plow rate: 20 mL/min; Gradient: 20% B to 20% B in 15 min; Wave Length: 220/254 nm; RT1 (min): 7.65; RT2 (min): 11.85; the second peak was product) to afford 536 (137.6 mg, 33.56%) as a yellow solid LCMS-536: (ES, m/z): [M+H]⁺ 495

NMR-536: (400 MHz, DMSO, ppm): δ 1.71-1.83 (m, 5H), 2.08-2.13 (m, 1H), 3.27-3.30 (m, 1H), 3.48 (s, 3H), 4.33-4.36 (d, 1H), 6.29-6.32 (t, 1H), 7.11-7.13 (d, 1H), 7.44 (s, 1H), 7.73-7.81 (m, 3H), 8.12 (s, 1H), 8.31-8.65 (m, 2H), 15.09 (d, 1H).

### Example 528. Synthesis of Compound 537

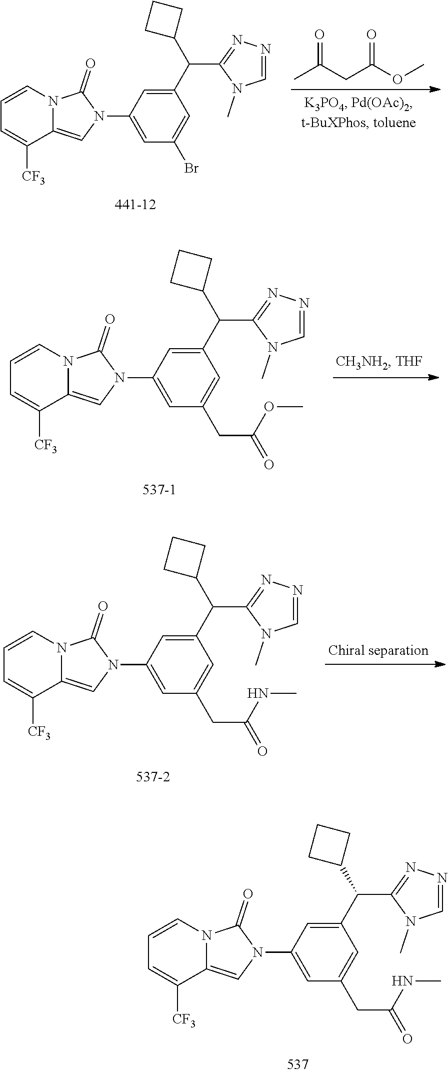

### 1. Synthesis of 537-1

Into a 100 mL 3-necked round-bottom flask were added 441-12 (2 g, 3,950 mmol, 1 equiv), toluene (20 mL), methyl acetoacetate (1.38 g, 11.850 mmol, 3 equiv), K₃PO₄ (2.52 g, 11,850 mmol, 3 equiv), Pd(OAc)₂ (0.09 g, 0.395 mmol, 0.1 equiv) and t-BuXPhos (0.34 g, 0,790 mmol, 0.2 equiv) at room temperature. The resulting mixture was stirred for overnight at 100° C. under nitrogen atmosphere. The mixture was allowed to cool down to room temperature. The reaction was quenched with water (20 m-L) at room temperature. The aqueous layer was extracted with EtOAc (2×20 mL). The resulting mixture was concentrated under vacuum. The residue was purified by Prep-TLC (PE/EA 5:1) to afford 537-1 (980 ng, 49.67%) as a yellow oil.

### 2. Synthesis of 537-2

Into a 20 mL sealed tube were added 537-1 (800 mg, 1.602 mmol, 1 equiv) and CH₃NH₂ (1M in THF) (8 mL) at room temperature. The resulting mixture was stirred for 2 days at room temperature. The resulting mixture was concentrated under vacuum. The residue was purified by Prep-TLC (CH₂Cl₂/MeOH 10:1) to afford 537-2 (220 mg, 27.55%) as a yellow oil.

### 3. Synthesis of 537

The 537-2 (120 mg) was purified by Chiral separation with the following conditions (Column: CHIRALPAK IH, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 20% B to 20% B in 9 min; Wave Length: 220/254 nm; RT1 (min): 6.78; RT2 (min): 8.01; The first peak is product; Sample Solvent: EtOH:DCM=1:1-HPLC; Injection Volume: 0.2 mL; Number of Runs: 10) to afford 537 (31.1 mg, 24.62%) as a yellow solid.

LC-MS-537: (ES, m/z): [M+H]⁺ 499

H-NMR-537: 1H NMR (400 MHz, DMSO-d6 ppm) δ 1.64-1.65 (d, 1H), δ 1.80 (s, 4H), δ 2.05-2.09 (d, 1H), δ 2.55-2.57 (d, 3H), δ 3.16-3.26 (m, 1H), δ 3.37-3.57 (m, 5H), δ 4.22-4.24 (d, 1H), δ 6.27-6.30 (t, 1H), δ 7.01-7.23 (m, 2H), δ 7.26 (s, 1H), δ 7.57-7.64 (d, 2H), δ 7.77-7.79 (d, 1H), δ 7.88-7.97 (m, 1H), δ 8.32 (s, 1H).

### Example 529. Synthesis of Compound 538

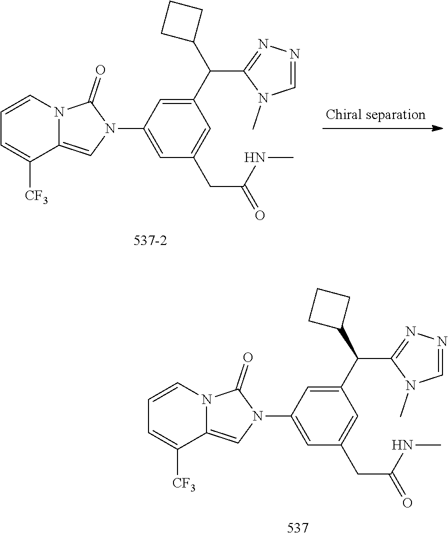 [SMILES: CNC(=O)Cc1cc(C(c2nncn2C)C2CCC2)cc(-n2cc3c(C(F)(F)F)cccn3c2=O)c1 ; CNC(=O)Cc1cc([C@H](c2nncn2C)C2CCC2)cc(-n2cc3c(C(F)(F)F)cccn3c2=O)c1]

### 1. Synthesis of 538

The 537-2 (120 mg) was purified by Chiral separation with the following conditions (Column: CHIRALPAK IH, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 20% B to 20% B in 9 min; Wave Length: 220/254 nm; RT1 (main): 6.78; RT2 (min): 8.01; The second peak was the product; Sample Solvent: EtOH:DCM=1:1-HPLC; Injection Volume: 0.2 mL; Number of Runs: 10) to afford 538 (31.8 mg, 25.18%) as a yellow solid.

LC-MS-538: (ES, m/z): [M+H]⁺ 499

H-NMR-538: 1H NMR (400 MHz, DMSO-d6 ppm) δ 1.77-1.79 (d, 1H), δ 1.80-1.86 (m, 4H), δ 2.05-2.09 (d, 1H), δ 2.55-2.57 (d, 3H), δ 3.16-3.26 (m, 1H), δ 3.37-3.43 (m, 5H), δ 4.22-4.24 (d, 1H), δ 6.27-6.30 (t, 1H), δ 7.11-7.23 (m, 2H), δ 7.25 (s, 1H), δ 7.57-7.63 (d, 2H), δ 7.77-7.79 (d, 1H), δ 7.96-7.97 (m, 1H), δ 8.32 (s, 1H).

### Example 530. Synthesis of Compound 539

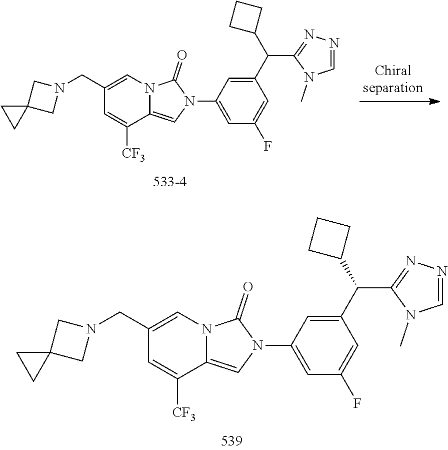 [SMILES: Cn1cnnc1C(c1cc(F)cc(-n2cc3c(C(F)(F)F)cc(CN4CC5(CC5)C4)cn3c2=O)c1)C1CCC1 ; Cn1cnnc1[C@H](c1cc(F)cc(-n2cc3c(C(F)(F)F)cc(CN4CC5(CC5)C4)cn3c2=O)c1)C1CCC1]

### 1. Synthesis of 539

The 533-4 (320 mg) was purified by Prep-Chiral-HPLC with the following conditions (Column: CHIRAL ART Amylose-SA, 2*25 ca, 5 μm; Mobile Phase A: Hex (0.5% 2M NH₃-MeOH), Mobile Phase B: EtOH:DCM=1:1; Flow rate: 20 mL/min; Gradient: 20% B to 20% B in 13.5 min; Wave Length: 220/254 nm; RT1 (min): 6.67; RT2 (min): 9.59; the first peak is product) to afford 539 (130.3 mg, 40.07%) as a yellow solid.

LC-MS-539: (ES, m/z): [M+H]⁺ 541

H-NMR-539: (400 MHz, DMSO-d6, δ ppm): 0.52 (s, 411), 1.67-1.82 (m, 5H), 2.04-2.08 (m, 1H), 3.18-3.24 (m, 1H), 3.37 (s, 4H), 3.43 (s, 3H), 3.54-3.64 (m, 2H), 4.30-4.33 (d, 1H), 7.04-7.06 (m, 2H), 7.42 (s, 1H), 7.69-7.72 (m, 3H), 8.35 (s, 1H).

### Example 531. Synthesis of Compound 540

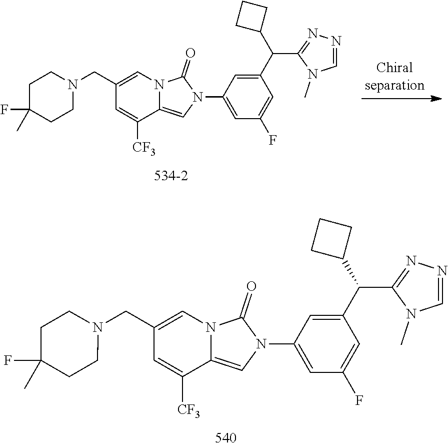 [SMILES: Cn1cnnc1C(c1cc(F)cc(-n2cc3c(C(F)(F)F)cc(CN4CCC(C)(F)CC4)cn3c2=O)c1)C1CCC1 ; Cn1cnnc1[C@H](c1cc(F)cc(-n2cc3c(C(F)(F)F)cc(CN4CCC(C)(F)CC4)cn3c2=O)c1)C1CCC1]

### 1. Synthesis of 540

The 534-2 (300 mg) was purified by Prep-Chiral-HPLC with the following conditions (Column: CHIRALPAK IC, 2*25 cm, 5 μm; Mobile Phase A: Hex (0.5% 2M NH₃-MeOH) Mobile Phase B: EtOH:DCM=1:1; Flow rate: 20 mL/min; Gradient: 60% B to 60% B in 10.5 min; Wave Length: 220/254 nm; RT1 (min): 6.43; RT2 (min): 834; the first peak is product) to afford 540 (52.1 mg, 17.37%) as a yellow solid.

LC-MS-540: (ES, m/z): [M+H]⁺ 559

H-NMR-540: (400 MHz, DMSO-d₆, δ ppm): 1.71-1.74 (m, 3H), 1.60-1.82 (m, 9H), 2.02-2.08 (m, 1H), 2.24-2.33 (m, 2H), 2.51-2.62 (m, 2H), 3.15-3.22 (m, 1H), 3.22 (s, 2H), 3.46 (s, 3H), 4.30-4.33 (d, 1H), 7.03-7.06 (m, 2H), 7.42 (s, 1H), 7.68-7.71 (m, 3H), 8.35 (s, 1H).

### Example 532. Synthesis of Compound 541

### 1. Synthesis of 541-1

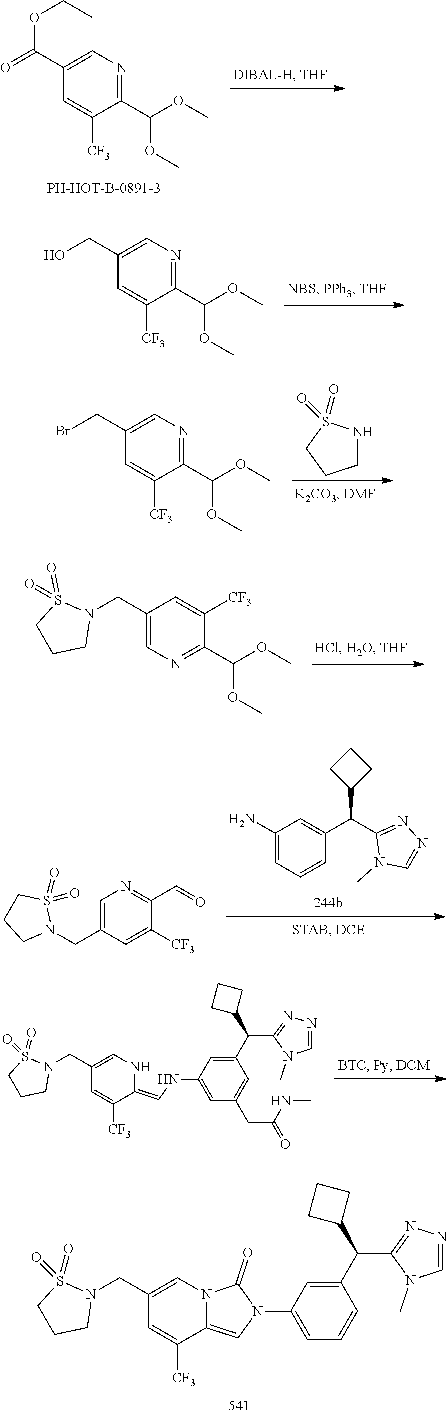

Into a 1 L 3-necked round-bottom flask were added 1-2 (25 g, 85.254 mmol, 1 equiv) and THF (250 mL) at 0° C. To the above mixture was added DIBAL-H (170.51 mL, 255,762 mmol, 3 equiv) dropwise over 5 min at 0° C. The resulting mixture was stirred for additional 2 h at room temperature. The reaction was quenched with sat. NH4Cl (aq.) (300 mL) at room temperature. The resulting mixture was extracted with EtOAc (3×300 mL). The combined organic layers were concentrated under reduced pressure. The residue was purified by silica gel column chromatography, eluted with PE/EA (5:1) to afford 541-1 (15.5 g, 68.76%) as a light brown oil.

### 2. Synthesis of 541-2

Into a 250 mL 3-necked round-bottom flask were added 541-1 (5 g, 19.904 mmol, 1 equiv), THF (100 mL) and PPh3 (10.44 g, 39.808 mmol, 2 equiv) at 0° C. To the above mixture was added NBS (7.09 g, 39.808 mmol, 2 equiv) in portions over 10 min at 0° C. The resulting mixture was stirred for additional 5 h at room temperature. The reaction was quenched with water (15 mL) at room temperature. The resulting mixture was extracted with EtOAc (3×150 mL). The combined organic layers were concentrated under reduced pressure. The residue was purified by silica gel column chromatography, eluted with PE/EA. (5:1) to afford 541-2 (6.8 g, 97.89%) as a yellow oil.

### 3. Synthesis of 541-3

Into a 40 mL sealed tube were added 541-2 (500 mg, 1.592 mmol, 1 equiv), DMF (5 ml), 1lambda6,2-thiazolidine-1,1-dione (482.13 mg, 3.980 mmol, 2.5 equiv) and K2CO3 (660.00 mg, 4.776 mmol, 3 equiv) at room temperature. The resulting mixture was stirred for overnight at 80° C. The reaction was quenched with water (40 mL) at room temperature. The resulting mixture was extracted with EtOAc (3×40 mL). The combined organic layers were concentrated under reduced pressure. The residue was purified by silica gel column chromatography, eluted with CH2Cl2/MeOH (50:1) to afford 541-3 (510 mg, 85.90%) as a light yellow oil.

### 4. Synthesis of 541-4

Into an 8 mL sealed tube were added 541-3 (450 mg, 1.270 mmol, 1 equiv), (1 M) HCl (5 mL) and THF (2 mL) at room temperature. The resulting mixture was stirred for 2 h at 80° C. The mixture was basified to pH 7 with saturated NaHCO₃ (aq.) (20 mL). The resulting mixture was extracted with EtOAc (3×20 mL). The combined organic layers were concentrated under reduced pressure. The residue was purified by Prep-TLC (CH2Cl2/MeOH 50:1) to afford 541-4 (410 mg, 99.49%) as a light yellow oil.

### 5. Synthesis of 541-5

Into a 20 ml sealed tube were added 541-4 (400 mg, 1.298 mmol, 1 equiv), DCE (5 mL), PH-244b (399.32 mg, 1.648 mmol, 1.27 equiv) and STAB (599.49 mg, 2,830 mmol, 2.18 equiv) at room temperature. The resulting mixture was stirred for 2 h at room temperature. The reaction was quenched with water (20 ml) at room temperature. The resulting mixture was extracted with EtOAc (3×20 mL). The combined organic layers were concentrated under reduced pressure. The residue was purified by Prep-TLC (CH2Cl2/MeOH 20:1) to afford 541-5 (300 mg, 38.35%) as a light yellow oil.

### 6. Synthesis of 541-0

Into a 40 mL sealed tube were added 541-5 (300 mg, 0.561 mmol, 1 equiv), pyridine (266.33 mg, 3.366 mmol, 6 equiv) and DCM (10 mL) at 0° C. To the above mixture was added triphosgene (66.61 m g, 0.224 mmol, 0.4 equiv) at 0° C. The resulting mixture was stirred for additional 5 mmi at 0° C. The mixture was basified to pH 7 with saturated NaHCO₃ (aq.) (10 mL). The resulting mixture was extracted with CH2Cl2 (3×10 mL). The combined organic layers were concentrated under reduced pressure. The crude product (200 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 32% B to 44% B in 8 min; Wave Length: 254 nm; RT1 (min): 7.7) to afford 541 (50.9 mg, 16.15%) as a light yellow solid.

LCMS 137-541: (ES, m/z): [M+H]+ 561

H-NMR17-541: (300 MHz, DMSO-d6, ppm): 51.70-1.81 (m, 5H), 52.08-2.09 (m, 1H), 52.20-2.27 (m, 2H), 63.16-3.28 (m, 5H), 63.31-3.43 (m, 3H), 63.96 (s, 2H), 64.25-4.28 (m, 2H), 66.97 (s, 1H), 67.19-7.21 (m, 1H), 67.36 (s, 1H), 67.42-7.46 (m, 1H), 67.69-7.73 (m, 2H), 67.84 (s, 1H), 68.33 (s, 1H).

### Example 533. Synthesis of Compound 542

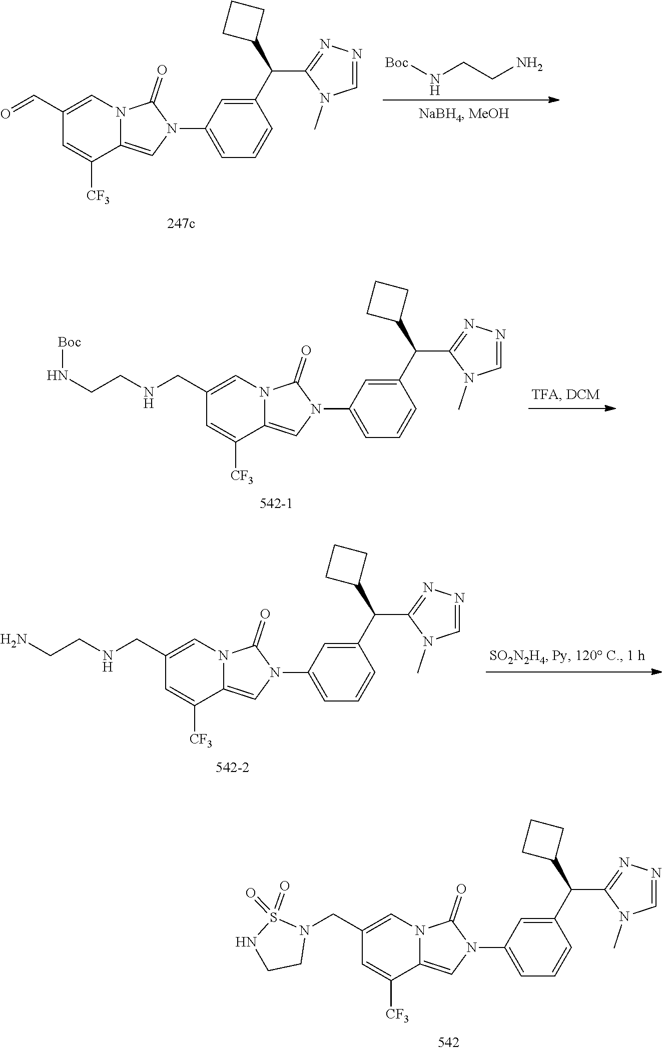 [SMILES: Cn1cnnc1[C@@H](c1cccc(-n2cc3c(C(F)(F)F)cc(C=O)cn3c2=O)c1)C1CCC1 ; CC(C)(C)OC(=O)NCCN ; Cn1cnnc1[C@@H](c1cccc(-n2cc3c(C(F)(F)F)cc(CNCCNC(=O)OC(C)(C)C)cn3c2=O)c1)C1CCC1 ; Cn1cnnc1[C@@H](c1cccc(-n2cc3c(C(F)(F)F)cc(CNCCN)cn3c2=O)c1)C1CCC1 ; Cn1cnnc1[C@@H](c1cccc(-n2cc3c(C(F)(F)F)cc(CN4CCNS4(=O)=O)cn3c2=O)c1)C1CCC1]

### 1. Synthesis of 542-1

A solution of 247c (3.5 g, 7,685 mmol, 1 equiv) in MeOH (35 mL) was treated with tert-butyl N-(2-aminoethyl) carbamate (6.16 g, 38.425 mmol, 5 equiv) for overnight at room temperature followed by the addition of NaBH₄ (0.87 g, 23.055 mmol, 3 equiv) at room temperature. The resulting mixture was stirred for 2 h at room temperature. The reaction was quenched by the addition of sat. NH₄HCl (aq.) (50 mL) at room temperature. The aqueous layer was extracted with CH₂Cl₂/MeOH (10:1) (3×50 mL). The resulting mixture was concentrated under reduced pressure. The residue was purified by silica gel column chromatography, eluted with CH₂Cl₂/MeOH (20:1) to afford 542-1 (1.15 g, 22.96%) as a yellow oil.

### 2. Synthesis of 542-2

Into a 80 mL sealed tube were added 542-1 (1.15 g, 1,918 mmol, 1 equiv), TFA (3 mL) and DCM (9 mL) at room temperature. The resulting mixture was stirred for 4 h at room temperature. The resulting mixture was concentrated under reduced pressure. The residue was purified by Prep-TLC (CH₂Cl₂/MeOH 15:1) to afford 542-2 (520 mg, 50.48%) as a yellow oil,

### 3. Synthesis of 542

Into a 40 mL sealed tube were added 542-2 (440 mg, 0.881 mmol, 1 equiv), sulfamide (101.57 mg, 1.057 mmol, 1.2 equiv) and pyridine (5 mL) at room temperature. The resulting mixture was stirred for 1 h at 120° C. The resulting mixture was concentrated under reduced pressure. The residue was purified by Prep-TLC (CH₂Cl₂/MeOH 15:1) to afford the crude product (80 mg), The crude product (80 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 45% B in 8 min; Wave Length: 254 nm; RT1 (min): 8.12) to afford 542-0 (26 mg, 5.24%) as a yellow solid.

LC-MS-542: (ES, m/z): [M+H]⁺ 562

H-NMR-542: (400 MHz, CDCl₃, ppm): δ 1.83-1.86 (m, 1H), δ 1.90-1.93 (m, 2H), δ 1.94-1.96 (d, 2H), δ 2.32-2.39 (m, 1H), δ 3.34-3.42 (m, 3H), δ 3.48 (s, 3H), δ 3.56-3.58 (m, 2H), δ 3.94-4.02 (m, 3H), δ 4.71 (s, 1H), δ 6.81 (s, 1H), δ 6.94 (s, 1H), δ 7.20-7.22 (d, 1H), δ 7.28 (s, 1H), δ 7.42-7.46 (m, 1H), δ 7.54-7.60 (s, 1H), δ 7.71 (s, 1H), δ 8.20 (s, 1H).

### Example 534. Synthesis of Compound 543

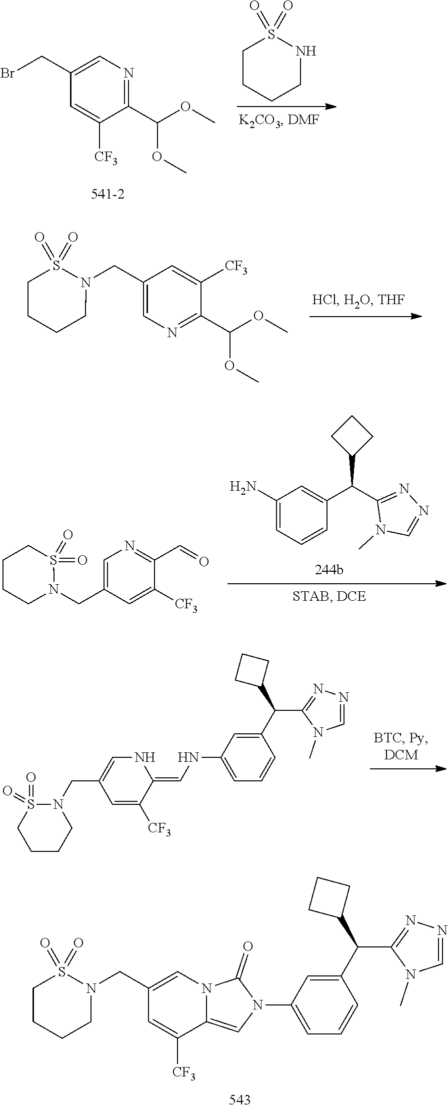

### 1. Synthesis of 543-1

Into a 40 mL sealed tube were added 541-2 (500 mg, 1.592 mmol, 1 equiv), DMF (5 mL), 1,2-thiazinane 1,1-dioxide (537.96 mg, 3.980 mmol, 2.5 equiv) and K₂CO₃ (660.00 mg, 4.776 mmol, 3 equiv) at room temperature. The resulting mixture was stirred for overnight at 80° C. The reaction was quenched with water (20 mL) at room temperature. The resulting mixture was extracted with EtOAc (3×40 mL). The combined organic layers were concentrated under reduced pressure. The residue was purified by silica gel column chromatography, elated with CH₂Cl₂/MeOH (50:1) to afford 543-1 (520 mg, 84.24%) as a light yellow oil.

### 2. Synthesis of 543-2

Into a 20 mL sealed tube were added 543-1 (500 mg, 1.357 mmol, 1 equiv), (1 M) HCl (5 mL) and THF (2 mL) at room temperature. The resulting mixture was stirred for 2 h at 80° C. The mixture was basified to pH 7 with saturated NaHCO₃ (aq.) (20 mL). The resulting mixture was extracted with EtOAc (3×20 mL). The combined organic layers were concentrated under reduced pressure. The residue was purified by Prep-TLC (CH₂Cl₂/MeOH 50:1) to afford 543-2 (420 mg, 91.21%) as a light yellow oil.

### 3. Synthesis of 543-3

Into a 20 mL sealed tube were added 543-3 (400 mg, 1.298 mmol, 1 equiv), DCE (5 mL), PH-244b (399.32 mg, 1.648 mmol, 1.27 equiv) and STAB (599.49 mg, 2.830 mmol, 2.18 equiv) at room temperature. The resulting mixture was stirred for 2 h at room temperature. The reaction was quenched with water (20 ml) at room temperature. The resulting mixture was extracted with EtOAc (3×20 ml). The combined organic layers were concentrated under reduced pressure. The residue was purified by Prep-TLC (CH₂Cl₂/MeOH 20:1) to afford 543-4 (200 mg, 38.35%) as a light yellow oil.

### 4. Synthesis of 543

Into a 20 mL sealed tube were added 543-4 (200 mg, 0.365 mmol, 1 equiv), pyridine (173.01 mg, 2.190 mmol, 6 equiv) and DCM (6 mL) at 0° C. To the above mixture was added triphosgene (43.27 mg, 0.146 mmol, 0.4 equiv) at 0° C. The resulting mixture was stirred for additional 5 min at 0° C. The mixture was basified to pH 7 with saturated NaHCO₃ (aq.) (10 mL). The resulting mixture was extracted with CH₂Cl₂ (3×10 mL). The combined organic layers were concentrated under reduced pressure. The crude product (200 mg) was purified by Prep-HPLC with the following conditions (Column: XBridge Shield RP18 OBD Column, 30*150 mm, 5 μm; Mobile Phase A: Water (10 mmol/L NH4HCO3), Mobile Phase B: ACN; Flow rate: 60 mL/in; Gradient: 30% B to 52% B in 8 min; Wave Length: 220 nm; RT1 (main): 7.52) to afford 543 (53.1 mg, 25.27%) as a light yellow solid.

LCMS 137-543: (ES, m/z): [M+H]⁺ 575

H-NMR17-543: (300 MHz, DMSO-d6, ppm): δ 1.68-1.70 (m, 2H), δ 1.70-1.81 (m, 5H), δ 2.01-2.09 (m, 3H), δ 3.16-3.31 (m, 5H), δ 3.43 (s, 3H), δ 4.10 (s, 2H), δ 4.25-4.28 (m, 1H), δ 6.92 (s, 1H), δ 7.19-7.21 (m, 1H), δ 7.37 (s, 1H), δ 7.43-7.46 (m, 1H), δ 7.69-7.73 (m, 2H), δ 7.85 (s, 1H), δ 8.33 (s, 1H).

### Example 535. Synthesis of Compound 544

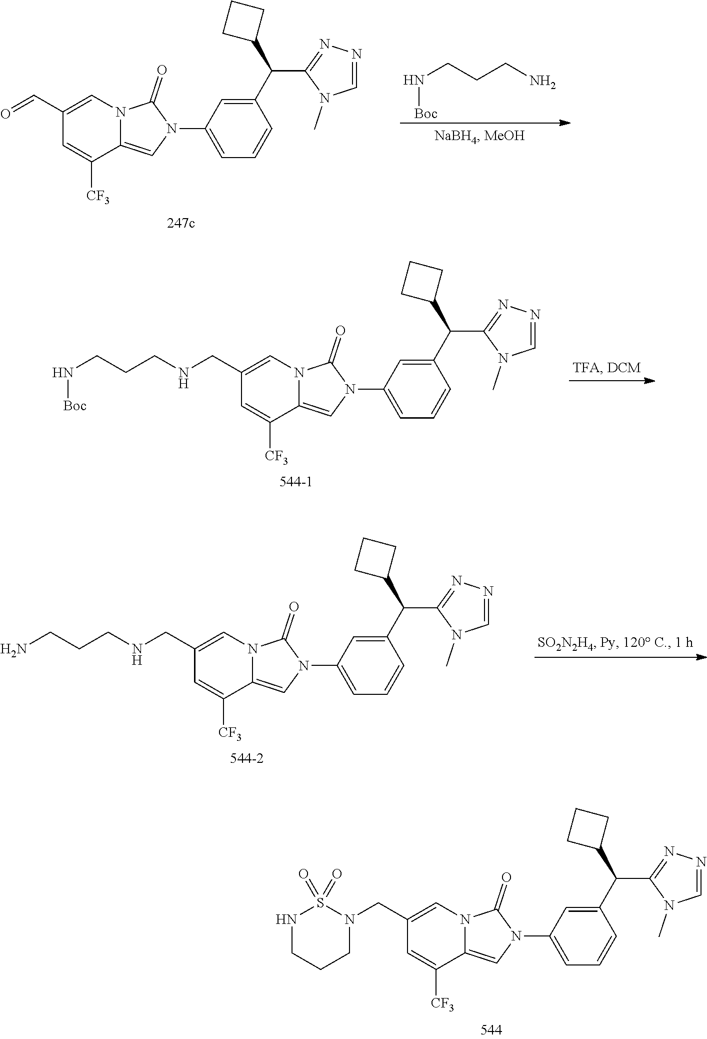 [SMILES: Cn1cnnc1[C@@H](c1cccc(-n2cc3c(C(F)(F)F)cc(C=O)cn3c2=O)c1)C1CCC1 ; CC(C)(C)OC(=O)NCCCN ; Cn1cnnc1[C@@H](c1cccc(-n2cc3c(C(F)(F)F)cc(CNCCCNC(=O)OC(C)(C)C)cn3c2=O)c1)C1CCC1 ; Cn1cnnc1[C@@H](c1cccc(-n2cc3c(C(F)(F)F)cc(CNCCCN)cn3c2=O)c1)C1CCC1 ; Cn1cnnc1[C@@H](c1cccc(-n2cc3c(C(F)(F)F)cc(CN4CCCNS4(=O)=O)cn3c2=O)c1)C1CCC1]

### 1. Synthesis of 544-1

A solution of 247c (3 g, 6.587 mmol, 1 equiv) in MeOH (30 mL) was treated with tert-butyl N-(3-aminopropyl) carbamate (5.74 g, 32,935 mmol, 5 equiv) for overnight at room temperature followed by the addition of NaBH4 (0.75 g, 19.761 mmol, 3 equiv) at room temperature. The resulting mixture was stirred for 2b at room temperature. The reaction was quenched by the addition of sat. NH4Cl (aq.) (50 mL) at room temperature. The aqueous layer was extracted with CH₂Cl₂/MeOH (10:1) (3×50 mL). The resulting mixture was concentrated under reduced pressure. The residue was purified by silica gel column chromatography, eluted with CH₂Cl₂/MeOH (20:1) to afford 544-1 (1.05 g, 23.90%) as a yellow oil.

### 2. Synthesis of 544-2

Into a 80 mL sealed tube were added 544-1 (1.05 g, 1.711 mmol, 1 equiv) TFA (3 mL) and DCM (9 mL) at room temperature. The resulting mixture was stirred for 4 h at room temperature. The resulting mixture was concentrated under reduced pressure. The residue was purified by Prep-TLC (CH₂Cl₂/MeOH 15:1) to afford 544-2 (550 mg, 58.21%) as a yellow oil.

### 3. Synthesis of 544

Into a 40 mL sealed tube were added 544-2 (550 mg, 1.071 mmol, 1 equiv), sulfamide (123.50 mg, 1.285 mmol, 1.2 equiv) and pyridine (5 mL) at room temperature. The resulting mixture was stirred for 1 h at 120° C. The resulting mixture was concentrated under reduced pressure. The residue was purified by Prep-TLC (CH₂Cl₂/MeOH 15:1) to afford the crude product (100 mg). The crude product (100 mg) was purified by Prep-HPLC with the following conditions (Column: XBridge Prep OBD C18 Column, 30*150 mm, 5 μm; Mobile Phase A: Water (10 mmol/L NIH1HCO3), Mobile Phase B: ACN; Flow rate: 60 mL/min; Gradient: 29% B to 54% B in 8 min; Wave Length: 220 nm; RT1 (min): 7.07) to afford 544 (38.1 mg, 6.16%) as a yellow solid.

LC-MS-544: (ES, m/z): [M+H]⁺ 576

H-NMR-544: (400 MHz, CDCl₃, ppm): δ 1.71-1.86 (m, 5H), δ 1.90-1.96 (m, 2H), δ 2.32-2.39 (m, 1H), δ 3.29-3.32 (m, 2H), δ 3.34-3.38 (m, 1H), δ 3.40-3.49 (m, 3H), δ 3.53-3.58 (d, 2H), δ 4.00-4.04 (m, 3H), δ 4.41 (s, 1H), δ 6.80 (s, 1H), δ 6.92 (s, 1H), δ 7.20-7.22 (d, 1H), δ 7.28 (s, 1H), δ 7.42-7.46 (m, 1H), δ 7.54-7.56 (d, 1H), δ 7.60-7.65 (d, 1H), δ 7.60 (s, 1H).

### Example 536. Synthesis of Compound 545

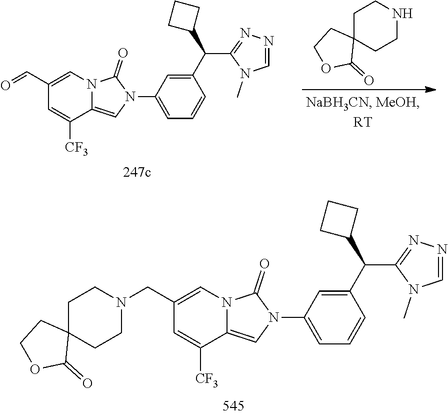 [SMILES: Cn1cnnc1[C@@H](c1cccc(-n2cc3c(C(F)(F)F)cc(C=O)cn3c2=O)c1)C1CCC1 ; O=C1OCCC12CCNCC2 ; Cn1cnnc1[C@@H](c1cccc(-n2cc3c(C(F)(F)F)cc(CN4CCC5(CCOC5=O)CC4)cn3c2=O)c1)C1CCC1]

### 1. Synthesis of 545

To a stirred solution of 247c (1000 mg, 2,196 mmol, 1 equiv) and 2-oxa-8-azaspiro[4.5]decan-1-one (681.52 mg, 4.392 mmol, 2 equiv) in MeOH (20 mL) were added NaBH₃CN (275.96 mg, 4.392 mmol, 2 equiv) at room temperature. The resulting mixture was stirred overnight at room temperature. The reaction was quenched with saturated NaHCO₃ (aq.) (50 mL) at room temperature. The resulting mixture was extracted with CH2Cl₂ (3×30 mL). The resulting mixture was concentrated under reduced pressure. The residue was purified by reverse flash chromatography with the following conditions: column, C18 silica gel; mobile phase, MeCN in Water (10 mmol/L NH4HCO₃), 30% to 60% gradient in 20 min; detector, UV 254 nm. This resulted in 545 (42.6 mg, 6.26%) as a yellow solid.

LC-MS-545: (ES, m/z): [M+H]⁺ 595

H-NMR-545: (400 MHz, DMSO-d6, δ ppm): 1.60-1.70 (m, 2H), 1.70-1.81 (m, 7H), 2.09-2.33 (m, 5H), 2.77-2.80 (m, 2H), 3.19-3.21 (m, 1H), 3.21-3.27 (m, 2H), 3.63 (s, 3H), 4.25-4.28 (m, 3H), 7.03 (s, 1H), 7.19-7.20 (m, 1H), 7.32 (s, 1H), 7.42-7.46 (m, 1H), 7.68-7.70 (m, 1H), 7.70-7.73 (m, 1H), 8.33 (s, 1H).

### Example 537. Synthesis of Compound 546

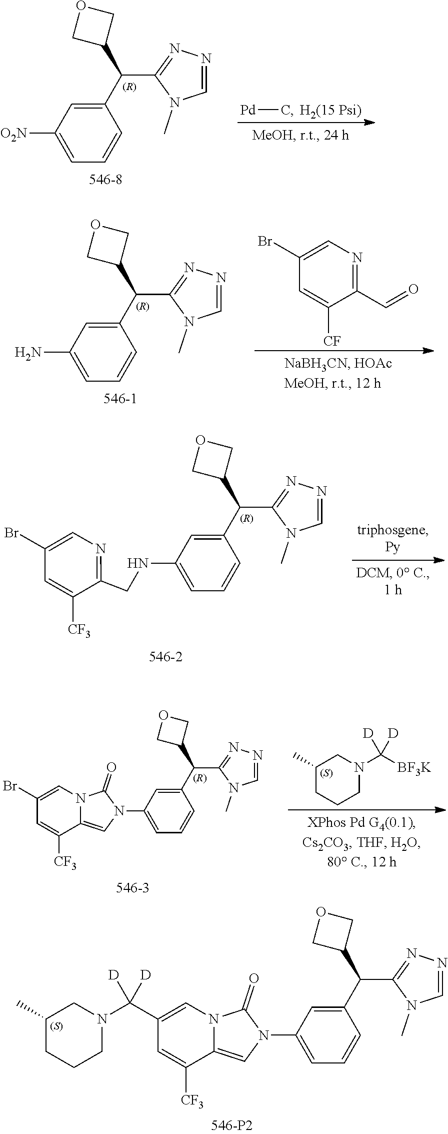

### 1. Synthesis of 546-1

To a solution of 546-8 (320 mg, 1.17 mmol, 1 equiv) in EtOAc (10 mL) was added Pd/C (50 mg, 10% purity) at 25° C. The suspension was degassed and purged with H2 for 3 times. The mixture was stirred under H₂ (15 Psi) at 25° C. for 24 h. The mixture was filtered and washed with MeOH (100 mL) and TI-IF (100 mL). The filtrate was concentrated in vacuum to get 546-1 (320 mg, crude) as a light yellow oil.

### 2. Synthesis of 546-2

To a solution of 546-1 (340 mg, 1.39 mmol, 1 equiv) in MeOH (5 mL) was added 5-bromo-3-(trifluoromethyl)pyridine-2-carbaldehyde (353 mg, 1.39 mmol, 1 equiv), AcOH (83.5 mg, 1.39 mmol, 1 equiv) at 25° C. and stirred for 1 h, NaBH₃CN (174 mg, 2.78 mmol, 2 equiv) was added and stirred for 11 h. The mixture was poured into H2O (50 mL) at 0° C., extracted with EtOAc (80 mL×3). The combined organic layer was washed by brine (50 mL×3), dried by Na2SO4, filtered and concentrated in vacuum to get a residue. The residue was purified by column (SiO2) with petroleum ether/EtOAc=3:1 to 0:1, then with DCM:MeOH=1:0 to 0:1 to give 546-2 (450 mg, 66%) as a light yellow solid,

### 3. Synthesis of 546-3

To a solution of 546-2 (200 mg, 414 umol, 1 equiv) in DCM (10 mL) was added Py (196 mg, 2.49 mmol, 6 equiv), bis(trichloromethyl) carbonate (62 mg, 208 umol, 0.5 equiv) at 0° C. and stirred for 1 h. The reaction mixture was diluted with Sat.NaHCO₃ (50 ml), extracted with DCM (50 mL×3). The combined organic layers were dried over Na2SO4, filtered and concentrated in vacuum to get a residue. The combined residue was purified by column (SiO₂) with petroleum ether/EtOAc=3:1 to 0:1, then with DCM:MeOH=1:0 to 0:1 to afford 546-3 (190 mg, 90%) as a light yellow solid.

### 4. Synthesis of 546

To a solution of 546-3 (390 mg, 767 umol, 1 equiv) in THF (6 mL), H₂O (1.5 mL) was added [dideuterio[(3S)-3-methyl-1-piperidyl]methyl]-trifluoro-boron;potassium hydride (339 mg, 1.53 mmol, 2 equiv), Cs₇CO₃ (749 mg, 2.30 mmol, 3 equiv), dicyclohexyl-[2-(2,4,6-triisopropylphenyl)-phenyl]phosphane;methanesulfonate;[2-[2-(methylamino)phenyl]phenyl]palladium(1+) (66.02 mg, 76.73 umol, 0.1 eq) under N₂. The suspension was degassed and purged with N₂ for 3 times. The mixture was heated to 80° C. and stirred for 12 h under N2. The same scale reaction was conducted with in parallel for 2 batches in total and work up with a small test scale (50 mg) together. The mixture was poured into H₂O (20 mL), extracted with DCM/MeOH (10:1, 20 mL×3). The combined organic layers were washed with brine (20 mL×2), dried over Na₂SO₄, filtered and concentrated under reduced pressure to give a residue. The residue was purified by column (SiO₂) with petroleum ether/EtOAc=1:1 to 0:1, then with DCM:MeOH=1:0 to 3:1 to get a crude product. The crude product was further purified by prep-HPLC (FA) (column: Phenomenex Luna C18 75*30 mm*3 um; mobile phase: [water (FA)-ACN]; B %: 1%-40%, 8 min, Wave Length: 220 nm; RT (min): 7.0 min) to get 546 (124.4 mg) as a light yellow solid.

MS-546: (ES, m/z): [M+H]⁺ 543.1

¹H-NMR-546: (400 MHz, MeOD) δ 8.39 (d, J=4.8 Hz, 2H), 7.86 (s, 1H), 7.69-7.65 (m, 2H), 7.53 (t, J=7.6 Hz, 1H), 7.30-7.28 (m, 1H), 7.20 (m, 1H), 7.12 (s, 1H), 4.95-4.92 (m, 1H), 4.83-4.81 (m, 1H), 4.67-4.61 (m, 2H), 4.43-4.40 (m, 1H), 4.01-3.97 (m, 1H), 3.50 (s, 3H), 3.26-3.20 (m, 2H), 2.56-2.53 (m, 1H), 2.28-2.23 (m, 1H), 1.89-1.67 (m, 4H), 1.15-1.11 (m, 1H), 0.97-0.95 (m, 3H).

### Example 538. Synthesis of Compound 547

### 1. Synthesis of 547-1

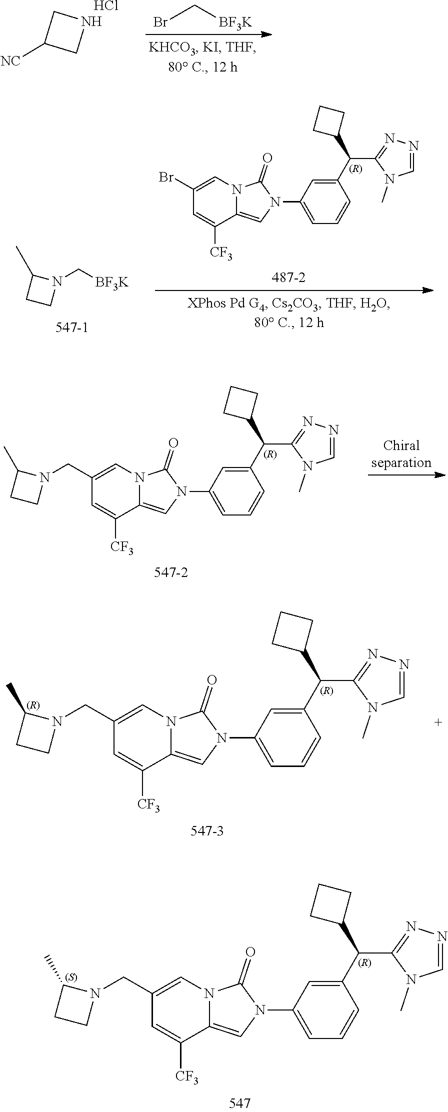

To a solution of 2-methylazetidine (0.5 g, 4.65 mmol, 1 equiv, HCl) in THF (5 mL) was added bromomethyl(trifluoro)boron;potassium hydride (933 mg, 4.65 mmol, 1 equiv), KI (77 mg, 464.76 umol, 0.1 equiv), KHCO₃ (93 mg, 9.30 mmol, 2 equiv). The mixture was heated to 80° C. and stirred at 80° C. for 12 hr. The mixture was concentrated under reduced pressure to give a crude. Then the crude was triturated with acetone (50 mL) at 50° C. for 2 h. The mixture was filtered and the filtrate was concentrated under reduced pressure to afford 547-1 (0.7 g, crude) as a light yellow solid.

### 2. Synthesis of 547-2

To a solution of 487-2 (400 mg, 790.02 umol, 1 equiv) in THF (6 mL) and H2O (1.5 mL) was added 547-1 (302 mg, 1.58 mmol, 2 equiv), dicesium;carbonate (772 mg, 2.37 mmol, 3 equiv), XPhosPd G4 (68 mg, 79.00 umol, 0.1 equiv). The suspension was degassed and purged with N2 for 3 times. Then the mixture was heated to 80° C. and stirred at 80° C. for 12 hr under N2 atmosphere. The reaction mixture was poured into water (20 mL), extracted with EtOAc (10 mL×3). The combined organic layer was washed with brine (30 mL), dried over anhydrous Na2SO4, filtered and the filtrate was concentrated in vacuum. The residue was purified by column chromatography (SiO2, Petroleum ether/Ethyl acetate=10/1 to 0/1) and (DCM: Methanol=100/1 to 0/1) to afford 547-2 (0.39 g, 96.53%) as a brown solid.

### 3. Synthesis of 547

547-2 (390 mg) was purified by Chiral separation with the following conditions (column: DAICEL CHIRALPAK IC (250 mm*30 mm, 10 um); mobile phase: [MeOH (0.1% IPAm)]; B %: 66%-66%, 30 min, Flow rate: 4 mL/min; Wave Length: 220/254 nm; RT2 (min): 2.342) to afford 547 (77 mg, 19.74%) as a yellow solid.

MS-547: (ES, m/z): [M+H]+ 511.3.

1H-NMR-547: (400 MHz, CDCl3) δ 8.02 (s, 1H), 7.62-7.60 (m, 2H), 7.55-7.53 (m, 1H), 7.42 (t, J=7.6 Hz, 1H), 7.17-7.15 (m, 1H), 6.96 (s, 1H), 6.75 (s, 1H), 3.98-3.95 (m, 1H), 3.49-3.35 (m, 6H), 3.25 (s, 2H), 2.81-2.78 (m, 1H), 2.38-2.31 (m, 1H), 2.10-2.09 (m, 1H), 1.95-1.69 (m, 5H), 1.26 (s, 1H), 1.16-1.15 (m, 3H).

### Example 539. Synthesis of Compound 548

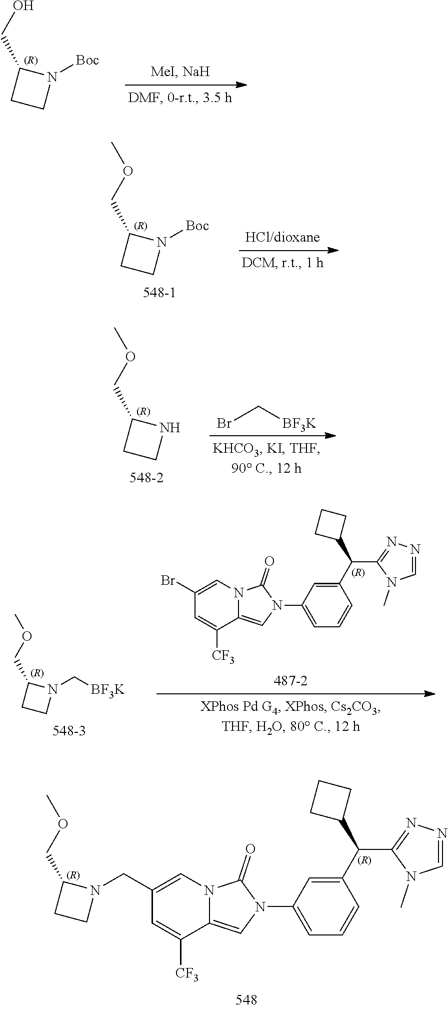

### 1. Synthesis of 548-1

To a solution of NaH (468 mg, 11.7 mmol, 60% purity, 1.22 equiv) in DMF (20 mL) was cooled to 0° C. under nitrogen atmosphere. Then a solution of tert-butyl (2R)-2-(hydroxymethyl)azetidine-1-carboxylate (1.8 g, 9.61 mmol, 1 equiv) in DMF (20 mL) was added dropwise. The mixture was stirred at 0° C. for 0-5 h. Then MeI (900 uL, 14.46 mmol, 1.5 equiv) was added at 0° C. The mixture was stirred at 20° C. for 3 h under nitrogen atmosphere. The mixture was quenched by saturated NH4Cl (300 mL) and extracted with EtOAc (100 mL×3). The combined organic layers were dried over anhydrous Na2SO4, filtered and the filtrate was concentrated under reduced pressure. The residue was purified by silica gel column chromatography eluted with CH2Cl2/MeOH (10:1) to afford 548-1 (1.8 g, 93%) as a colorless oil.

1H-NMR-548-1: (400 MHz, CHLOROFORM-d) δ 4.29-4.18 (m, 1H), 3.79-3.70 (m, 2H), 3.63-3.55 (m, 1H), 3.50-3.44 (m, 1H), 3.34 (s, 3H), 2.20-2.03 (m, 2H), 1.37 (s, 9H)

### 2. Synthesis of 548-2

To a solution of 548-1 (1.2 g, 5.96 mmol, 1 equiv) in DCM (12 mL) was added HCl/dioxane (4 M, 8.94 mL, 6 equiv). The mixture was stirred at 20° C. for 1 h. The mixture was concentrated under N2 (20° C.) to afford 548-2 (603 mg, crude) as a yellow oil.

### 3. Synthesis of 548-3

A mixture of 548-2 (603 mg, 5.96 mmol, 1 equiv), potassium;bromomethyl(trifluoro) boranuide (1.2 g, 5.96 mmol, 1 equiv), KHCO3 (1.9 g, 11.9 mmol, 2 equiv) and KI (98.9 mg, 596 umol, 0.1 equiv) in THF (10 mL) was stirred at 90° C. for 12 h under nitrogen atmosphere. The mixture was concentrated under reduced pressure. The residue was triturated with acetone (50 ml) at 35° C. for 30 min. Then the mixture was filtered, and the filtrate was concentrated under reduced pressure to afford 548-3 (1 g, crude) as a white solid.

### 4. Synthesis of 548

To a solution of 487-2 (300 mg, 592 umol, 1 equiv) and 548-3 (655 mg, 2.96 mmol, 5 equiv) in THF (8 mL) and H2O (2 ml) was added XPhos (28.2 mg, 59.2 umol, 0.1 equiv), Cs2CO3 (579 mg, 1.78 mmol, 3 equiv) and dicyclohexyl-[2-(2,4,6-triisopropylphenyl)phenyl]phosphane; methanesulfonate;[2-[2-(methylamino)phenyl]phenyl]palladium(1+) (50.98 mg, 59.25 umol, 0.1 equiv). The mixture was stirred at 80° C. for 12 h under nitrogen atmosphere. The reaction mixture was extracted with water (120 mL) and EtOAc (40 mL×3). The combined organic layers were dried over anhydrous Na2SO4, filtered and the filtrate was concentrated under reduced pressure to give a residue. The crude product (100 mg) was purified by prep-HPLC with the following conditions (column: Phenomenex Luna C18 200*40 mm*10 um; Mobile Phase A: Water (0.2% formic acid), Mobile Phase B: ACN; Flow rate: 50 mL/min; Gradient: 5% B to 45% B in 8 min; Wave Length: 220 nm; RT1 (min): 7.5) to afford 548 (39.5 mg, 39.29%) as a yellow solid.

MS-548: (ES, m/z): [M+H]+ 541.3

1H-NMR-548: (400 MHz, METHANOL-d4) δ 8.36 (s, 1H), 7.71-7.59 (m, 3H), 7.48 (t, J=8.0 Hz, 1H), 7.25 (d, J=7.6 Hz, 1H), 7.08 (d, J=12.8 Hz, 2H), 4.28 (d, J=10.8 Hz, 1H), 3.60 (d, J=13.2 Hz, 1H), 3.55-3.48 (m, 4H), 3.44 (d, J=4.4 Hz, 1H), 3.42-3.39 (m, 1H), 3.37-3.33 (m, 2H), 3.28 (s, 3H), 3.07-2.99 (m, 1H), 2.29-2.19 (m, 1H), 2.15-1.81 (m, 7H), 1.80-1.71 (m, 1H), 1.36-1.20 (m, 0.4H).

### Example 540. Synthesis of Compound 549

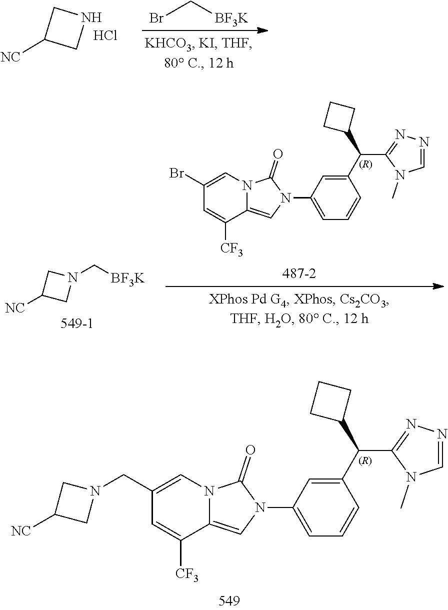

### 1. Synthesis of 549-1

To a solution of azetidine-3-carbonitrile;hydrochloride (1 g, 8.43 mmol, 1 equiv) and bromomethyl(trifluoro)boron;potassium hydride (1.69 g, 8.43 mmol, 1 equiv) in THF (10 mL) was added KHCO3 (1.69 g, 16.8 mmol, 2 equiv) and KI (140 mg, 843 umol, 0.1 equiv). The mixture was stirred at 80° C. for 12 h under nitrogen atmosphere. The mixture was concentrated under reduced pressure to give a residue. Then the residue was triturated with acetone at 35° C. for 2 h and filtered and filtrate was concentrated under reduced pressure to afford 549-1 (460 mg, crude) as a white solid.

### 2. Synthesis of 549

To a solution of 549-1 (399 mg, 1.98 mmol, 4 equiv) and 487-2 (250 mg, 493 umol, 1 equiv) in THF (16 mL) and 120 (4 mL) was added XPhos (23.5 mg, 49.4 umol, 0.1 equiv), dicyclohexyl-[2-(2,4,6-triisopropylphenyl)phenyl]phosphane;methanesulfonate;[2-[2-(methylamino)phenyl] phenyl]palladium(1+) (42.5 mg, 49.4 umol, 0.1 equiv) and Cs2CO3 (482.6 mg, 1.48 mmol, 3 equiv). The mixture was stirred at 80° C. for 12 h under nitrogen atmosphere. The reaction mixture was extracted with NaHCO₃ (90 mL) and EtOAc (30 mL×3). The combined organic layers were dried over anhydrous Na2SO4, filtered and the filtrate was concentrated under reduced pressure to give a residue. The residue was purified by silica gel column chromatography eluted with CH2Cl2/MeOH (10:1) to give a crude product. The crude product (225 mg) was purified by prep-HPLC with the following conditions (column: Phenomenex Luna C18 200*40 mm*10 um; Mobile Phase A: Water (0.2% formic acid), Mobile Phase B: ACN; Flow rate: 50 mL/min; Gradient: 5% B to 45% B in 8 min; Wave Length: 220 nm; RT1 (min): 7.5) to afford 549 (37.3 mg, 14.19%) as a yellow solid,

MS-549: (ES, m/z): [M+H]+ 522.2

1H-NMR-549: (400 MHz, METHANOL-d4) δ 8.36 (s, 1H), 7.68 (s, 1H), 7.65-7.61 (m, 2H), 7.48 (t, J=7.6 Hz, 1H), 7.26 (d, J=7.6 Hz, 1H), 7.10 (s, 1H), 7.01 (s, 1H), 4.28 (d, J=10.8 Hz, 1H), 3.63-3.58 (m, 2H), 3.54 (s, 3H), 3.47-3.41 (m, 5H), 2.28-2.21 (m, 1H), 1.97-1.84 (m, 5H), 1.79-1.73 (m, 1H).

### Example 541. Synthesis of Compound 550_P1&P2

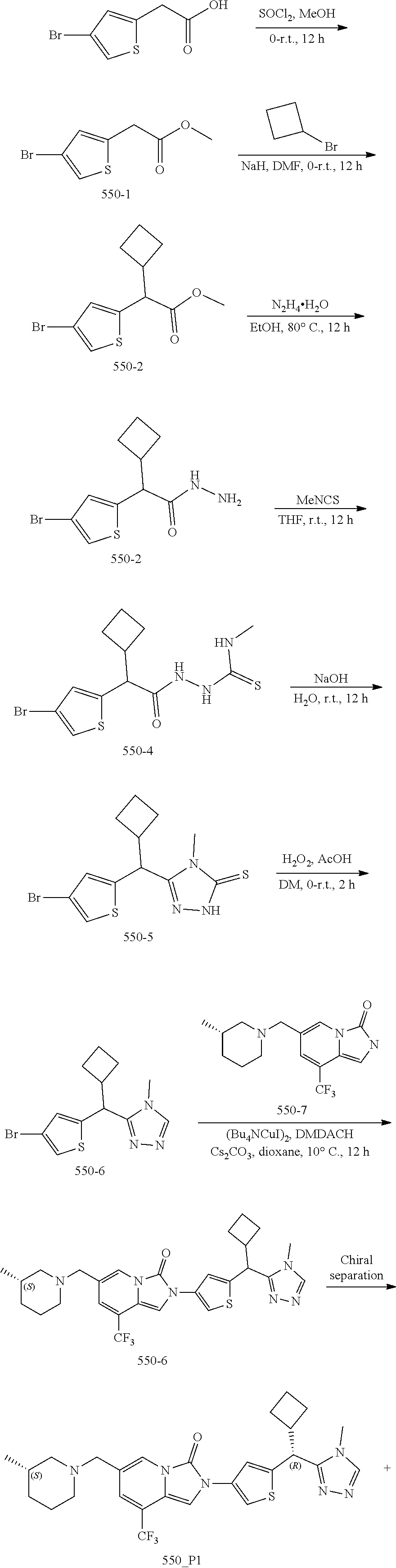

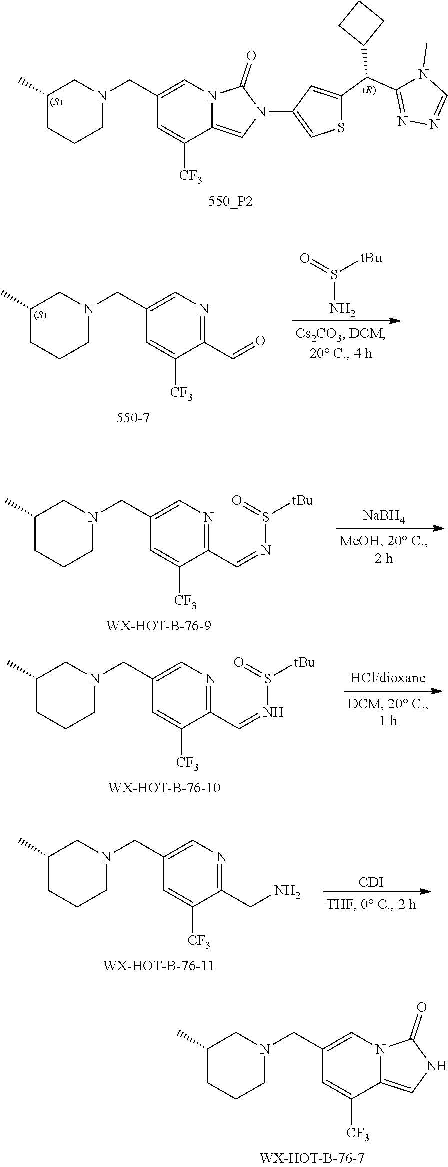

### 1. Synthesis of 550-1

To a solution of 2-(4-bromo-2-thienyl)acetic acid (9 g, 407 mmol, 1 equiv) in MeOH (120 mL) was added SOCl2 (14.7 mL, 203 mmol, 5 equiv) at 0° C., the mixture was stirred at 25° C. for 12 h under nitrogen atmosphere. The reaction mixture was concentrated in vacuum. The residue was purified by silica gel column chromatography eluted with EtOAc/PE (5:1) to afford 550-1 (8.8 g, 92%) as a red oil.

1H-NMR-550-1: (400 MHz, CHLOROFORM-d) δ 7.04 (d, J=1.6 Hz, 1H), 6.80 (s, 1H), 3.72 (s, 2H), 3.66 (s, 3H)

### 2. Synthesis of 550-2

To a solution of NaH (1.68 g, 42.1 mmol, 60% purity, 1.1 equiv) in DMF (90 mL) was added 550-1 (9 g, 38.2 mmol, 1 equiv). The mixture was cooled to 0° C., then bromocyclobutane (10.8 mL, 114 mmol, 3 equiv) was added to the mixture at 0° C. The result mixture was stirred at 25° C. for 12 hr under nitrogen atmosphere. The reaction mixture was diluted with Sat. NH4Cl (100 mL), extracted with EtOAc (2×80 mL). The combined organic layers were dried over anhydrous Na2SO4, filtered and concentrated in vacuum. The residue was purified by silica gel column chromatography eluted with EtOAc/PE (5:1) to afford 550-2 (8.4 g, 76%) as a yellow oil.

1H-NMR-550-2: (400 MHz, CHLOROFORM-d) δ 7.02 (d, J=1.6 Hz, 1H), 6.77 (d, J=0.8 Hz, 1H), 3.73-3.69 (m, 1H), 3.62 (s, 3H), 2.88-2.72 (m, 1H), 2.12-2.00 (m, 1H), 1.97-1.87 (m, 1H), 1.81-1.70 (m, 3H), 1.66-1.57 (m, 1H)

### 3. Synthesis of 550-3

To a solution of 550-2 (2 g, 6.92 mmol, 1 equiv) in EtOH (20 mL) was added N2H4·H2O (10.3 mL, 207 mmol, 98% purity, 30 equiv), the mixture was stirred at 80° C. for 12 hr. The reaction mixture was concentrate in vacuum to remove EtOH (20 mL). Then the mixture was diluted with water (20 mL), and extracted with CH2Cl2 (20 mL×3). The combined organic layers were dried over Na2SO4, filtered and concentrated under reduced pressure to give a residue to afford 550-3 (1.6 g, crude) as a white solid.

### 4. Synthesis of 550-4

To a solution of 550-3 (1.6 g, 5.53 mmol, 1 equiv) in THF (20 mL) was added methylimino (thioxo)methane (756 uL, 11.0 mmol, 2 equiv). The mixture was stirred at 25° C. for 4 hr. The reaction mixture was diluted with water (15 m-L), extracted with EtOAc (15 mL×3). The mixture was filtered and the filter cake was concentrated in vacuum to afford 550-4 (1 g, 50%) as a white solid.

1H-NMR-550-4: (400 MHz, DMSO-d6) δ 10.01 (s, 1H), 9.32 (s, 1H), 7.69 (s, 1H), 7.50 (d, J=1.6 Hz, 1H), 6.97 (d, J=1.2 Hz, 1H), 3.77 (d, J=10.4 Hz, 1H), 2.86 (d, J=4.0 Hz, 3H), 2.74-2.64 (m, 1H), 1.98-1.89 (m, 2H), 1.87-1.80 (m, 1H), 1.79-1.73 (m, 2H), 1.72-1.66 (m, 1H)

### 5. Synthesis of 550-5

To a solution of NaOH (883 mg, 22.1 mmol, 8 equiv) in H2O (5.3 mL) was added 550-4 (1 g, 2.76 mmol, 1 equiv). The mixture was stirred at 25° C. for 2 hr. The reaction mixture was diluted with water (10 mL), adjusted pH to 3 by 1N HCl. The mixture was filtered and the filter cake was concentrated in vacuum to afford 550-5 (900 mg, 95%) as a white solid.

### 6. Synthesis of 550-6

A solution of 550-5 (0.9 g, 2.61 mmol, 1 equiv) in DCM (30 mL) was cooled to 0° C. Then a solution of H₂O₂ (1.00 mL, 10.46 mmol, 30% purity, 4 equiv) in AcOH (1.2 mL, 20.9 mmol, 8 equiv) was added to the mixture at 0° C. The result mixture was stirred at 25° C. for 2 hr. The solvent was removed under reduced pressure, and then diluted with water (30 mL) and adjusted pH to 12 by 2N NaOH. The mixture was then extracted with CH2Cl2 (25 mL×3). The combined organic layers were dried over anhydrous Na2SO4, filtered and concentrated under reduced pressure to afford 550-6 (800 mg, crude) as a yellow oil.

1H-NMR-550-6: (400 MHz, METHANOL-d4) δ 8.38 (s, 1H), 7.30 (d, J=1.6 Hz, 1H), 6.89 (d, J=0.8 Hz, 1H), 4.52 (d, J=10.8 Hz, 1H), 3.59 (s, 3H), 3.28-3.17 (m, 1H), 2.21-2.12 (m, 1H), 2.08-2.02 (m, 1H), 1.97-1.84 (m, 4H)

### 7. Synthesis of 550-9

To a solution of 1-2 (6 g, 20.96 mmol, 1 equiv) in DCM (80 mL) was added 2-methylpropane-2-sulfinamide (2.54 g, 20.96 mmol, 1 equiv), Cs2CO3 (13.66 g, 41.92 mmol, 2 equiv). The mixture was stirred at 20° C. for 4 h. The reaction mixture was poured into water (60 mL), extracted with CH2Cl2 (20 ml×3). The combined organic layers were washed with brine (60 mL), dried over anhydrous Na2SO4, filtered and the filtrate was concentrated in vacuum. The residue was purified by silica gel column chromatography eluted with CH2Cl2/MeOH (10:1) to afford 550-9 (6 g, 73.51%) as a yellow oil.

1H-NMR-550-9: (400 MHz, CHLOROFORM-d) δ 8.96-8.95 (m, 1H), 8.87 (s, 1H), 8.09 (s, 1H), 3.59 (s, 2H), 2.77-2.70 (m, 2H), 2.01-1.95 (m, 1H), 1.73-1.56 (m, 5H), 1.30 (s, 9H), 0.94-0.85 (m, 4H).

### 8. Synthesis of 550-10

To a solution of 550-9 (3 g, 7.70 mmol, 1 equiv) in MeOH (40 mL) was added NaBH4 (1.55 g, 40.9 mmol, 5.32 equiv). The mixture was stirred at 20° C. for 2 h. The reaction mixture was poured into water (100 ml), extracted with EtOAc (50 mL×3). The combined organic layer was washed with brine (100 mL), dried over anhydrous Na2SO4, filtered and concentrated under reduced pressure to give a crude product. The residue was purified by silica gel column chromatography eluted with CH2Cl2/MeOH (5:1) to afford 550-10 (2.8 g, 92.8%) as a yellow oil.

1H-NMR: (400 MHz, CHLOROFORM-d) δ 8.63 (s, 1H), 7.93 (s, 1H), 5.04 (t, J=5.2 Hz, 1H), 4.57 (d, J=5.2 Hz, 2H), 3.51 (s, 2H), 2.75-2.69 (m, 2H), 1.95-1.90 (m, 1H), 1.72-1.60 (m, 4H), 1.27 (s, 9H), 0.92-0.84 (m, 4H).

### 9. Synthesis of 550-11

To a solution of 550-10 (5.3 g, 13.5 mmol, 1 equiv) in DCM (60 mL) was added HCl/dioxane (4 M, 16.9 mL, 5 equiv). The mixture was stirred at 20° C. for 1 h. The reaction mixture was concentrated under reduced pressure to give a residue and adjusted pH=8 with ion resin. The mixture was filtered and filtrated concentrated in vacuum to afford 550-11 (3.9 g, crude) as a red oil.

1H-NMR: (400 MHz, METHANOL-d4) δ 8.76 (s, 1H), 8.13 (s, 1H), 4.26 (s, 2H), 3.62 (s, 2H), 2.79-2.76 (m, 2H), 2.02-1.95 (m, 1H), 1.75-1.57 (m, 5H), 0.93-0.85 (m, 4H).

### 10. Synthesis of 550-7

To a solution of 550-11 (3.9 g, 13.5 mmol, 1 equiv) in THF (40 mL) was added CDI (2.2 g, 13.5 mmol, 1 equiv) at 0° C. The mixture was stirred at 0° C. for 2 h. The reaction mixture was poured into water (60 mL), extracted with EtOAc (30 ml×3). The combined organic layer was washed with brine (80 ml), dried over anhydrous Na2SO4, filtered and concentrated under reduced pressure to give a crude product. The residue was purified by silica gel column chromatography eluted with CH2Cl2/MeOH (5:1) to afford 550-7 (2.7 g, 63.5%) as a yellow solid.

1H-NMR 8: (400 MHz, METHANOL-d4) δ 7.59 (s, 1H), 7.01 (s, 1H), 6.68 (s, 1H), 3.29 (s, 2H), 2.87-2.80 (m, 2H), 1.97-1.90 (m, 1H), 1.74-1.53 (m, 5H), 0.96-0.86 (m, 4H).

### 11. Synthesis of 550-8

To a solution of 550-7 (188 mg, 599 umol, 1.1 equiv) in dioxane (10 mL) was added 550-6 (170 mg, 545 umol, 1 equiv), Cs2CO3 (355 ng, 1.09 mmol, 2 equiv), iodocopper; tetrabutylammonium;diiodide (61 mg, 54.45 umol, 0.1 equiv), N1,N2-dimethylcyclohexane-1,2-diamine (15.49 mg, 108.89 umol, 0.2 equiv) in glove box. The mixture was heated to 110° C. and stirred for 12 h at 110° C. The same scale reaction was conducted with in parallel for 2 batches in total and work up together. The mixture was washed with NaHCO₃ (20 m L) and extracted with DCM (10 mL×3). The combined organic layer was washed with brine (40 mL) and dried over anhydrous Na2SO4, filtered and concentrated in vacuum. The residue was purified by silica gel column chromatography eluted with CH2Cl2/MeOH (10:1) to afford 550-8 (350 mg, 59.02%) as a yellow solid.

### 12. Synthesis of 550_P1&P2

550-8 (350 mg) was purified by Chiral separation with the following conditions (column: DAICEL CHIRALCEL OD (250 mm*30 mm, 10 um); mobile phase: [Neu-IPA]; B %: 38%-38%, 9 min, Flow rate: 2.5 mL/min; Wave Length: 220/254 nm; RT1 (min): 3.961; RT2 (min): 4.249) to afford 550 P1 (117.3 mg, 33.51%) as a yellow solid and 550_P2 (118.9 mg, 33.97%) as a yellow solid.

MS-550_P1: (ES, m/z): [M+H]+ 545.2.

1H-NMR-550_P1: (400 MHz, DMSO-d6) δ 8.36 (s, 1H), 7.82 (d, J=1.6 Hz, 1H), 7.65-7.63 (m, 2H), 7.40 (s, 1H), 6.99 (s, 1H), 4.57 (d, J=10.4 Hz, 1H), 3.53 (s, 3H), 3.23-3.13 (m, 3H), 2.75-2.71 (m, 2H), 2.03-1.79 (m, 6H), 1.77-1.57 (m, 5H), 1.44 (d, J=12.0 Hz, 1H), 0.85-0.81 (m, 4H).

MS-550_P2: (ES, m/z): [M+H]+ 545.2. 1H-NMR-550_P2: (400 MHz, DMSO-d6) δ 8.36 (s, 1H), 7.82 (d, J=1.6 Hz, 1H), 7.65-7.63 (m, 2H), 7.41 (s, 1H), 6.99 (s, 1H), 4.57 (d, J=10.4 Hz, 1H), 3.53 (s, 3H), 3.23-3.13 (m, 3H), 2.75-2.66 (m, 2H), 2.02-1.81 (m, 6H), 1.75-1.59 (m, 5H), 1.59-1.45 (m, 1H), 0.85-0.81 (m, 4H).

### Example 542. Synthesis of Compound 551

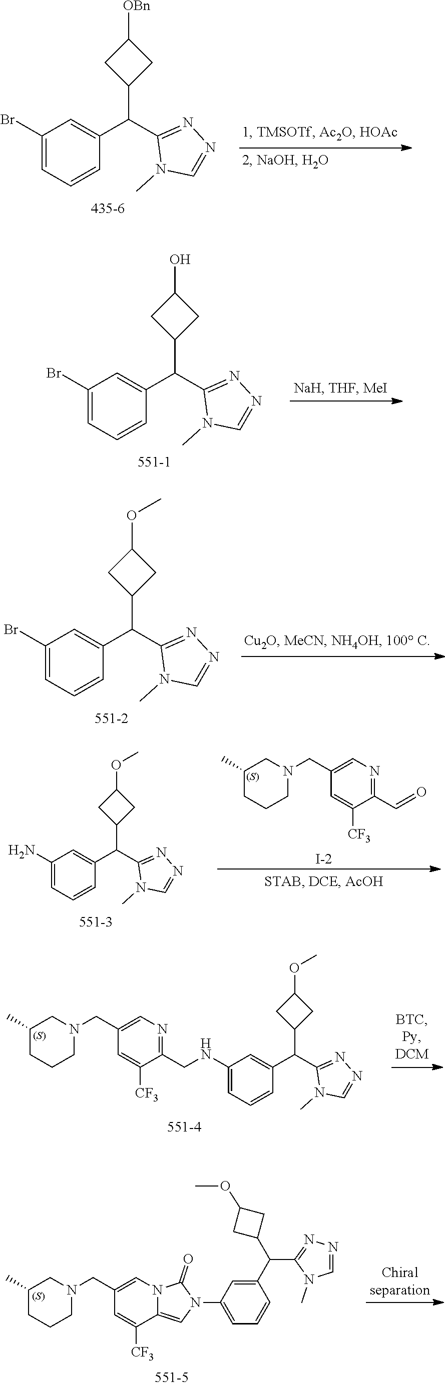

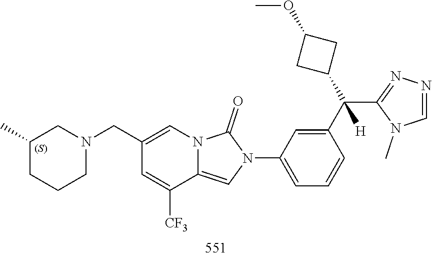

### 1. Synthesis of 551-1

To a stirred solution of 435-6 (67 g, 16249 mmol, 1 equiv) in HOAc (70 mL) and Ac₂O (15 mL) was added TMSOTf (7222.72 mg, 32.498 mmol, 2 equiv) at room temperature. The resulting mixture was stirred for 2 h at room temperature. The resulting mixture was concentrated under reduced pressure. The residue was dissolved in MeOH (20 mL). To the above mixture was added NaOH (65.00 mL, 64.996 mmol, 4 equiv, 1M). The resulting mixture was stirred for additional 4 h at room temperature. The resulting mixture was extracted with CH₂Cl₂/MeOH=10/1 (3×50 mL). The combined organic layers were dried over anhydrous MgSO₄. After filtration, the filtrate was concentrated under reduced pressure. This resulted in 551-1 (4.4 g, 84.04%) as a white solid.

### 2. Synthesis of 551-2

To a stirred solution of 551-1 (2 g, 6.207 mmol, 1 equiv) in THF (20 mL) was added NaH (0.30 g, 7.448 mmol, 1.2 equiv, 60%) at 0° C. under nitrogen atmosphere. The resulting mixture was stirred for 0.5 h at 0° C. under nitrogen atmosphere. To the above mixture was added MeI (1.32 g, 9.310 mmol, 1.5 equiv) at 0° C. The resulting mixture was stirred for additional overnight at room temperature. The reaction was quenched by the addition of sat. NH₄Cl (aq.) (60 mL). The resulting mixture was extracted with EtOAc (3×50 mL). The combined organic layers were concentrated under reduced pressure. The residue was purified by silica gel column chromatography, eluted with CH₂Cl₂/MeOH (30:1) to afford 551-2 (1.8 g, 86.25%) as a white solid.

### 3. Synthesis of 551-3

Into a 50 mL pressure tank reactor were added 551-2 (1.8 g, 5.353 mmol, 1 equiv), MeCN (30 mL), NH40H (15 mL) and Cu2O (0.31 g, 2.141 mmol, 0.4 equiv) at room temperature. The resulting mixture was stirred for overnight at 100° C. The resulting mixture was diluted with DCM (100 mL). The resulting mixture was filtered, the filter cake was washed with DCM (2×10 mL). The filtrate was concentrated under reduced pressure. The residue was purified by silica gel column chromatography, eluted with CH₂Cl₂/MeOH (20:1) to afford 551-3 (1.2 g, 82.30%) as a grey solid.

### 4. Synthesis of 551-4

To a stirred solution of 551-3 (1.2 g, 4.406 mmol, 1 equiv) and 1-2 (1.51 g, 5.287 mmol, 1.2 equiv) in DCE (20 mL) was added STAB (1.87 g, 8.812 mmol, 2 equiv). The resulting mixture was stirred for overnight at room temperature. The resulting mixture was diluted with water (20 mL). The resulting mixture was extracted with CH₂Cl₂/MeOH=10/1 (2×30 mL). The combined organic layers were concentrated under reduced pressure. The residue was purified by silica gel column chromatography, eluted with CH₂Cl₂/MeOH (20:1) to afford 551-5 (1.6 g, 66.92%) as a white solid.

### 5. Synthesis of 551-6

To a stirred solution of 551-5 (1.58 g, 2.912 mmol, 1 equiv) and Pyridine (1.15 g, 14.560 mmol, 5 equiv) in DCM (40 mL) was added Triphosgene (0.30 g, 1.019 mmol, 0.35 equiv) at 0° C. under nitrogen atmosphere. The resulting mixture was stirred for 2 h at 0° C. under nitrogen atmosphere. The reaction was quenched by the addition of sat. NaHCO₃ (aq.) (50 mL). The resulting mixture was extracted with CH₂Cl₂/MeOH=10/l (2×50 mL). The combined organic layers were concentrated under reduced pressure. The residue was purified by reverse flash chromatography with the following conditions: column, C18 silica gel; mobile phase, MeCN in Water (10 mmol/L NH₄CO₃), 10% to 65% gradient in 30 min; detector, UV 254 nm. This resulted in 551-5 (1 g, 60.40%) as a yellow solid.

### 6. Synthesis of 551

551-5 (1 g) was purified by Prep-Chiral-SFC with the following conditions (Column: CHIRAL ART Cellulose-SB, 3*25 cm, 5 um; Mobile Phase A: CO2, Mobile Phase B: ETOH (0.1% 2M NH₃-MEOH); Flow rate: 100 mL/min; Gradient: isocratic 45% B; Column Temperature (° C.): 35; Back Pressure (bar): 100; Wave Length: 220 nm; RT1 (min): 242; RT2 (min): 4.47; the first peak was desired product) to afford crude product (420 ng). The crude product (420 mag) was purified by Prep-Chiral-HPLC with the following conditions (Column: CHIRALPAK IH, 2*25 cm, 5 um; Mobile Phase A: Hex (0.5% 2M NH₃-MeOH), Mobile Phase B: EtOH:DCM=1:1; Flow rate: 20 mL/mi; Gradient: 20% B to 20% B in 7.5 min; Wave Length: 220/254 nm; RT1 (min): 4.83; RT2 (min): 6.49; the first peak was product) to afford 551 (305.3 mg, 30.53%) as a yellow solid.

LCMS-551: (ES, m/z): [M+H]⁺ 569

NMR-551: (400 MHz, CD₃OD, δ ppm): 0.90-0.99 (m, 4H), 1.61-1.81 (m, 7H), 1.96-2.01 (m, 1H), 2.19-2.27 (m, 1H), 2.58-2.63 (m, 1H), 2.79-2.92 (m, 3H), 3.24 (s, 3H), 3.32 (s, 2H), 3.54 (s, 3H), 3.79-3.84 (t, 1H), 4.24-4.26 (d, 1H), 7.13 (s, 2H), 7.26-7.28 (d, 1H), 7.49-7.51 (t, 1H), 7.65-7.70 (m, 3H), 8.38 (s, 1H).

### Example 543. Synthesis of Compound 552

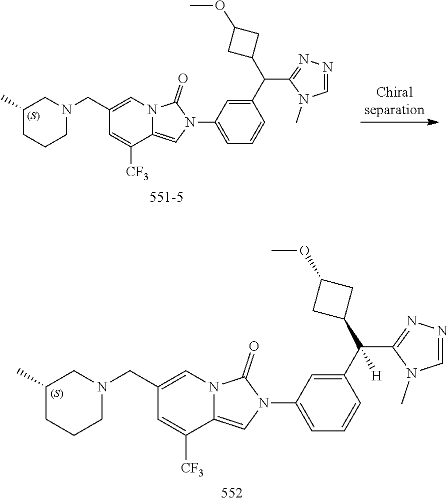

### 1. Synthesis of 552

551-5 (1 g) was purified by Prep-Chiral-SFC with the following conditions (Column: CHIRAL ART Cellulose-SB, 3*25 cm, 5 μm; Mobile Phase A: CO2, Mobile Phase B: ETOH (0.1% 2M NH3-MEOH); Flow rate: 100 mL/min; Gradient: isocratic 45% B; Column Temperature (° C.): 35; Back Pressure (bar): 100; Wave Length: 220 nm; RT1 (min): 2.42; RT2 (min): 4.47; the first peak was product) to afford crude product (380 mg). The crude product (380 mg) was purified by Prep-Chiral-HPLC with the following conditions (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 ml/min; Gradient: 20% B to 20% B in 29 mini; Wave Length: 220/254 nm; RT1 (min): 18.19; RT2 (min): 26.89; the second peak was product) to afford 552 (24.3 mg, 2.43%) as a yellow solid

LCMS-552: (ES, m/z): [M+H]⁺ 569

NMR-552: (400 MHz, CD₃OD, δ ppm): 0.90-0.97 (m, 4H), 1.61-1.79 (m, 5H), 1.99-2.01 (m, 3H), 2.15-2.17 (m, 1H), 2.32-2.35 (m, 1H), 2.87-2.93 (m, 3H), 3.24 (s, 3H), 3.28-3.32 (m, 1H), 3.36 (s, 2H), 3.55 (s, 3H), 4.09-4.11 (t, 1H), 4.31-4.34 (d, 1H), 7.13-7.15 (d, 2H), 7.28-7.30 (d, 1H), 7.50-7.54 (t, 1H), 7.66-7.72 (m, 3H), 8.38 (s, 1H).

### Example 544. Synthesis of Compound 553

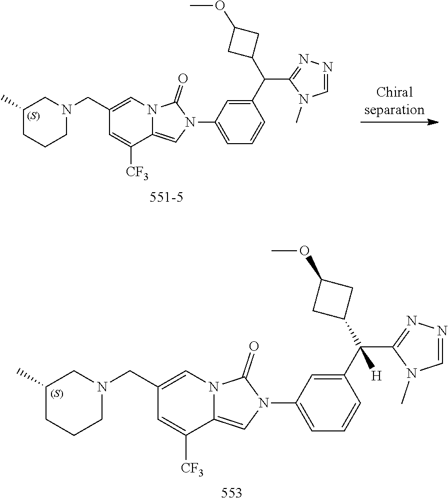

### 1. Synthesis of 553

551-5 (1 g) was purified by Prep-Chiral-SFC with the following conditions (Column: CHIRAL ART Cellulose-SB, 3*25 cm, 5 um; Mobile Phase A: CO₂, Mobile Phase B: ETOH (0.1% 2M NH₃-MEOH); Flow rate: 100 mL/min; Gradient: isocratic 45% B; Column Temperature (° C.): 35; Back Pressure (bar): 100; Wave Length: 220 nm; RT1 (min): 242; RT2 (min): 4.47; the second peak was product) to afford crude product (380 mg). The crude product (380 mg) was purified by Prep-Chiral-HPLC with the following conditions (Column: CHIRALPAK IF, 2*25 cm, 5 um; Mobile Phase A: Hex (0.5% 2M NH₃-MeOH), Mobile Phase B: EtOH:DCM=1:1; Flow rate: 20 mL/min; Gradient: 20% B to 20% B in 29 min; Wave Length: 220/254 nm; RT1 (min): 18.19; RT2 (min): 26.89; the second peak was product) to afford 553 (25.6 mg, 2.56%) as a yellow solid

LCMS: (ES, m/z): [M+H]⁺ 569. NMR: (400 MHz, CD₃OD, δ ppm): 0.90-0.97 (m, 4H), 1.61-1.78 (m, 5H), 1.01-2.03 (m, 3H), 2.15-2.18 (m, 1H), 2.28-2.35 (m, 1H), 2.87-2.93 (m, 3H), 3.24 (s, 3H), 3.28-3.32 (m, 1H), 3.36 (s, 2H), 3.55 (s, 3H), 4.06-4.10 (m, 1H), 4.31-4.34 (d, 1H), 7.13-7.15 (d, 2H), 7.28-7.30 (d, 1H), 7.50-7.54 (t, 1H), 7.66-7.71 (m, 3H), 8.38 (s, 1H).

### Example 545. Synthesis of Compound 554

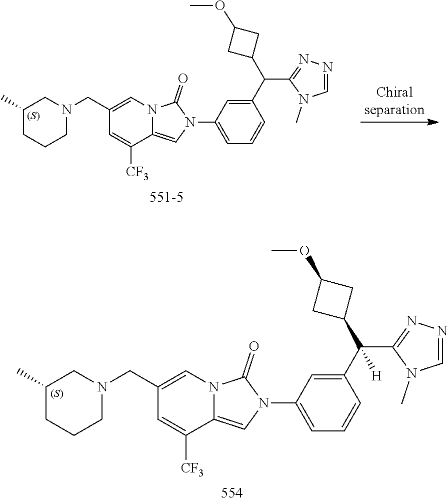

### 1. Synthesis of 554

551-5 (1 g) was purified by Prep-HPLC with the following conditions (Column: CHIRAL ART Celluose-SB, 3*25 cm, 5 μm; Mobile Phase A: CO₂, Mobile Phase B: ETOH (0.1% 2M NH3-MEOH); Flow rate: 100 mL/min; Gradient: isocratic 45% B; Column Temperature (° C.): 35; Back Pressure (bar): 100; Wave Length: 220 nm; RT1 (min): 2.42; RT2 (min): 4.47; second peak was desired product) to afford crude product (420 mg). The crude product (420 mg) was purified by Prep-HPLC with the following conditions (Column: CHIRALPAK IH, 2*25 cm, 5 μm; Mobile Phase A: Hex (0.5% 2M NH3-MeOH), Mobile Phase B: EtOH:DCM=1:1; Flow rate: 20 mL/min; Gradient: 20% B to 20% B in 7.5 min; Wave Length: 220/254 nm; RT1 (min.): 4.83; RT2 (min): 6.49; first peak was product) to afford 554 (285.7 mg, 28.57%) as a yellow solid

LCMS-554: (ES, m/z): [M+H]⁺ 569

NMR-554: 0.90-0.99 (m, 4H), 1.61-1.81 (m, 7H), 1.96-2.01 (m, 1H), 2.21-2.24 (m, 1H), 2.58-2.62 (m, 1H), 2.81-2.91 (m, 3H), 3.24 (s, 3H), 3.32 (s, 2H), 3.54 (s, 3H), 3.81-3.84 (t, 1H), 4.24-4.26 (d, 1H), 7.13 (s, 2H), 7.26-7.28 (d, 1H), 7.49-7.53 (t, 1H), 7.66-7.68 (m, 3H), 8.38 (s, 1H).

### Example 546. Synthesis of Compound 555

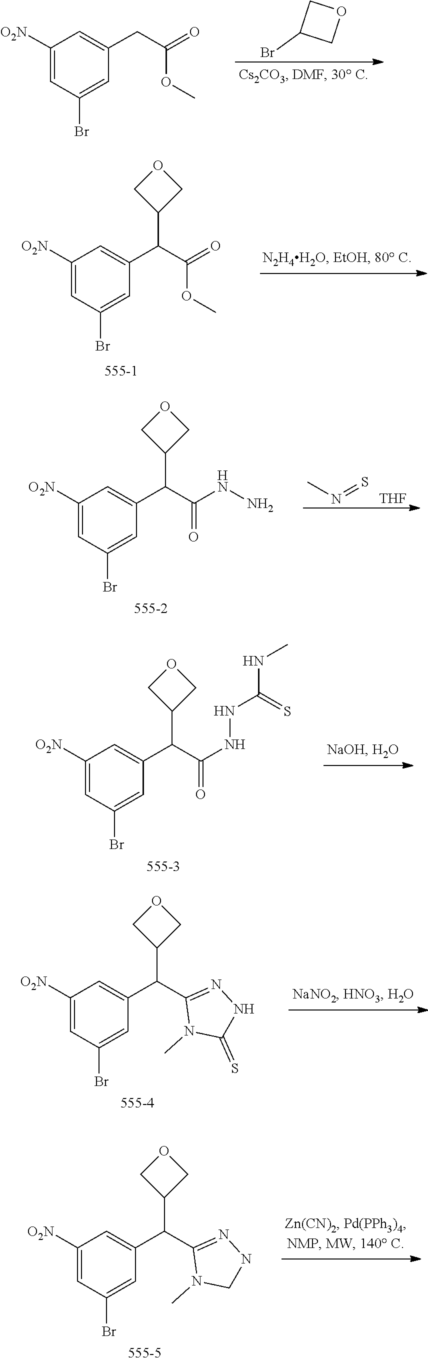

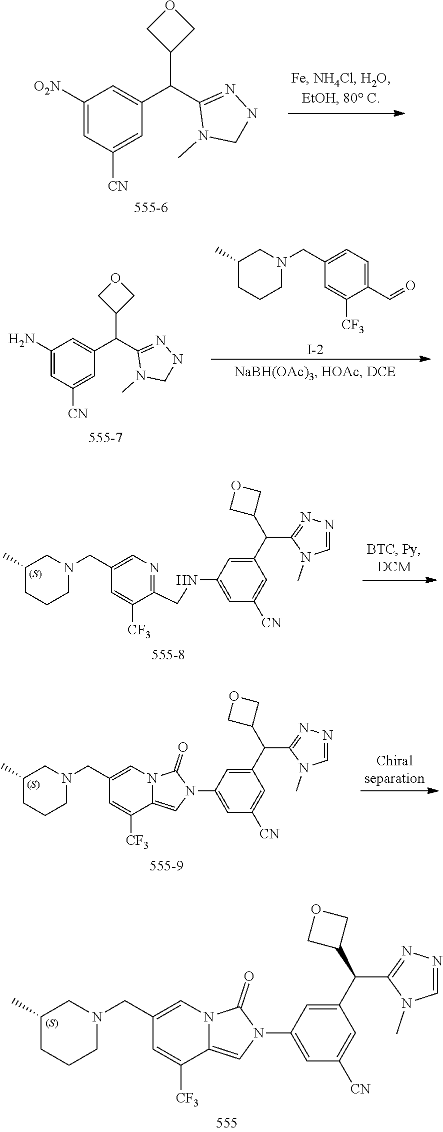

### 1. Synthesis of 555-1

Into a 250 mL 3-necked round-bottom flask was added 555-1 (9 g, 32.838 mmol, 1 equiv), DMF (100 mL) and Cs₂CO₃ (53.50 g, 164.190 mmol, 5 equiv) at 0° C. The resulting mixture was stirred for 1 h at 0° C. under nitrogen atmosphere. To the above mixture was added 3-bromooxetane (13-50 g, 98.514 mmol, 3 equiv) at room temperature. The resulting mixture was stirred overnight at room temperature. The reaction was quenched with saturated NH₄Cl (aq.) (500 mL) at room temperature. The resulting mixture was extracted with EtOAc (3×500 mL). The combined organic layers were concentrated under reduced pressure. The residue was purified by silica gel column chromatography, eluted with PE/EA (50:1) to afford methyl 555-2 (6.2 g, 54.33%) as a light yellow solid,

### 2. Synthesis of 555-2

Into a 100 mL 3-necked round-bottom flask was added 555-1 (6.2 g, 18.780 mmol, 1 equiv), EtOH (70 mL) and NH2NH2·H2O (9.40 g, 187.800 mmol, 10 equiv) at room temperature. The resulting mixture was stirred overnight at 80° C. under nitrogen atmosphere. The reaction was quenched with water (100 mL) at room temperature. The resulting mixture was extracted with EtOAc (3×100 mL). The combined organic layers were concentrated under reduced pressure. The residue was purified by silica gel column chromatography, eluted with DCM/MeOH (50:1) to afford 555-2 (3.2 g, 49.03%) as a brown solid,

### 3. Synthesis of 555-3

Into a 100 mL 3-necked round-bottom flask were added 555-2 (3.2 g, 9.693 mmol, 1 equiv), tetrahydrofuran (35 mL) and methyl isothiocyanate (1.77 g, 24,232 mmol, 2.5 equiv) at room temperature. The resulting mixture was stirred overnight at room temperature. The reaction was quenched with water at room temperature. The precipitated solids were collected by filtration and washed with water (2×20 mL). This result in 555-3 (3.3 g, 80.21%) as a light yellow solid.

### 4. Synthesis of 555-4

Into a 100 mL 3-necked round-bottom flask were added 555-3 (3.3 g, 8.184 mmol, 1 equiv), H₂O (80 mL) and NaOH (3.27 g, 81840 mmol, 10 equiv) at room temperature. The resulting mixture was stirred overnight at room temperature. The mixture was acidified to pH 6 with HCl (aq.). The precipitated solids were collected by filtration and washed with water (3×20 mL). This result in 555-4 (3 g, 90.40%) as a light yellow solid.

### 5. Synthesis of 555-5

Into a 100 mL 3-necked round-bottom flask were added 555-4 (3 g, 7.787 mmol, 1 equiv), 1H₂O (35 mL), NaNO2 (5.37 g, 77.870 mmol, 10 equiv) at 0° C. To the above mixture was added HNO₃ (78 mL, 77.870 mmol, 10 equiv, 1 M) dropwise at 0° C. The resulting mixture was stirred overnight at room temperature. The mixture was basified to pH 7 with saturated NaHCO₃ (aq.). The resulting mixture was extracted with EtOAc (3×100 mL). The combined organic layers were concentrated under reduced pressure. The residue was purified by silica gel column chromatography, eluted with CH₂Cl₂/MeOH (50:1) to afford 555-5 (2 g, 69.08%) as a light yellow solid,

### 6. Synthesis of 555-6

To a solution of 555-5 (2 g, 5.663 mmol, 1 equiv) and Zn(CN)2 (1.99 g, 16.948 mmol, 2.99 equiv) in NMP (30 mL) was added Pd(PPh₃)₄ (0.65 g, 0.562 mmol, 0.10 equiv) at room temperature under nitrogen atmosphere. The final reaction mixture was irradiated with microwave radiation for 3 h at 140° C. The reaction was quenched with 1M LiCl (aq.) (200 mL) at room temperature. The resulting mixture was extracted with EtOAc (6×50 mL). The combined organic layers were concentrated under reduced pressure. The residue was purified by Prep-TLC (DCM/MeOH 20:1) to afford 555-6 (1 g, 56.05%) as a light yellow oil.

### 7. Synthesis of 555-7

Into a 100 mL 3-necked round-bottom flask were added 555-6 (1 g, 3.341 mmol, 1 equiv), EtOH (20 mL), H₂O (5 mL), NH₄Cl (1.79 g, 33.410 mmol, 10 equiv) and Fe (0.56 g, 10,023 mmol, 3 equiv) at room temperature. The resulting mixture was stirred overnight at 80° C. under nitrogen atmosphere. The resulting mixture was filtered, the filter cake was washed with EtOAc (2×10 mL). The filtrate was diluted with water (100 mL) at room temperature. The resulting mixture was extracted with EtOAc (5×50 mL). The combined organic layers were concentrated under reduced pressure. The residue was purified by Prep-TLC (DCM/MeOH 20:1) to afford 555-7 (500 mg, 52.79%) as a light yellow solid.

### 8. Synthesis of 555-8

To a stirred 555-7 (500 mg 1,856 mmol, 1 equiv) and 1-2 (637.85 mg, 2.228 mmol, 1.2 equiv) in DCE (10.00 mL) at room temperature. The resulting mixture was stirred overnight at room temperature. To the above mixture was added STAB (1573.96 mg, 7.426 mmol, 2 equiv) at room temperature. The resulting mixture was stirred for additional 4 h at room temperature. The reaction was quenched with saturated NaHCO₃ (aq.) (80 mL) at room temperature. The aqueous layer was extracted with DCM (3×50 mL). The residue was purified by Prep-TLC (DCM/MeOH 15:1) to afford 555-8 (300 mg, 39.96%) as a white solid.

### 9. Synthesis of 555-9

To a stirred solution of 555-8 (300 mg, 0.556 mmol, 1 equiv) and Pyridine (263.86 mg, 3.336 mmol, 6.00 equiv) in DCM (4 mL) was added Triphosgene (64.34 mg, 0.217 mmol, 0.39 equiv) at room temperature. The resulting mixture was stirred for 10 min at room temperature. The reaction was quenched with saturated NaHCO₃ (aq.) (80 mL) at room temperature. The aqueous layer was extracted with DCM (3×40 mL). The resulting mixture was concentrated under vacuum. The residue was purified by Prep-TLC (DCM/MeOH 15:1) to afford 555-9 (220 mg, 67.16%) as a yellow solid.

### 10. Synthesis of 555

The 555-9 (220 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 45% B to 45% B in 10 min; Wave Length: 220/254 nm; RT1 (min): 5.93; RT2 (min): 7.87; the second peak is product) to afford 555 (58.7 mg, 26.68%) as a yellow solid.

LC-MS-555: (ES, m/z): [M+H]⁺ 558

H-NMR-555: (400 MHz, DMSO-d₆, 5 ppm): 0.80-0.93 (m, 4H), 1.44-1.78 (m, 5H), 1.94-1.98 (m, 1H), 2.74-2.83 (m, 2H), 3.21-3.30 (m, 2H), 3.46 (s, 3H), 3.93-4.01 (m, 1H), 4.27-4.30 (m, 1H), 4.46-4.52 (m, 2H), 4.71-4.75 (m, 1H), 4.90-4.93 (d, 1H), 7.03 (s, 1H), 7.56 (s, 1H), 7.65-7.70 (m, 2H), 8.20 (s, 1H), 8.30 (s, 1H), 8.40 (s, 1H).

### Example 547. Synthesis of Compound 556

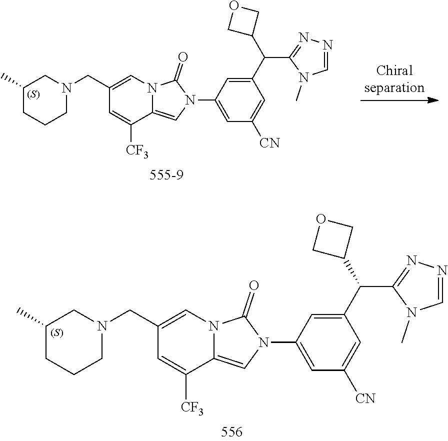

### 1. Synthesis of 556

The 555-9 (220 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 45% B to 45% B in 10 min; Wave Length: 220/254 nm; RT1 (min): 5.93; RT2 (min): 7.87; the frit peak is product) to afford 556 (51.6 mg, 2345%) as a yellow solid.

LC-MS-556: (ES, m/z): [M+H]⁺ 559

H-NMR-556: (400 MHz, DMSO-d₆, 5 ppm): 0.80-0.93 (m, 4H), 1.44-1.78 (m, 5H), 1.94-1.98 (m, 1H), 2.74-2.83 (m, 2H), 3.21-3.30 (m, 2H), 3.46 (s, 3H), 3.93-4.01 (m, 1H), 4.27-4.30 (m, 1H), 4.46-4.52 (m, 2H), 4.71-4.75 (m, 1H), 4.90-4.93 (d, 1H), 7.03 (s, 1H), 7.56 (s, 1H), 7.65-7.70 (m, 2H), 8.20 (s, 1H), 8.30 (s, 1H), 8.40 (s, 1H).

### Example 548. Synthesis of Compound 557

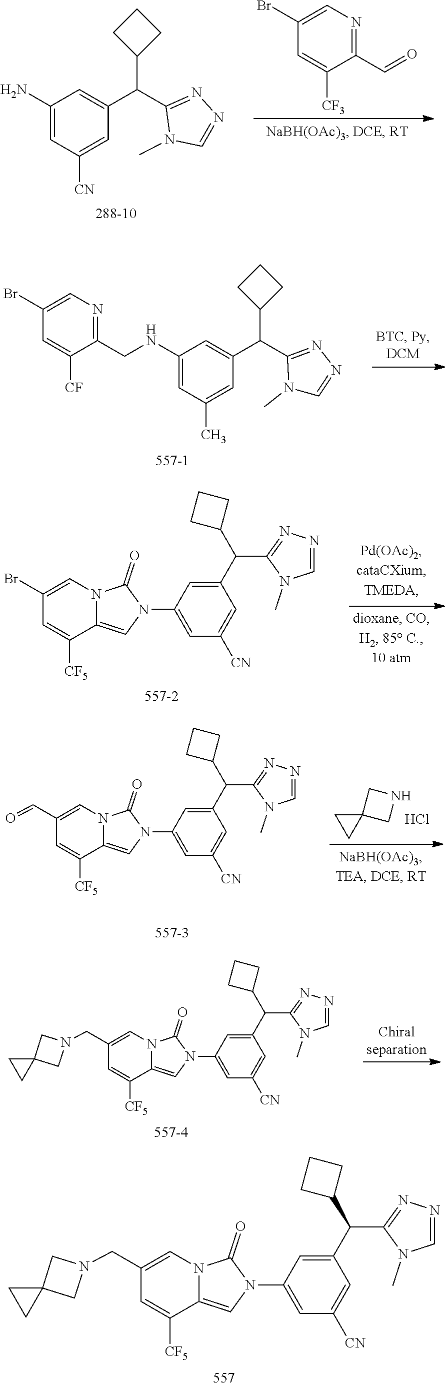

### 1. Synthesis of 557-1

To a stirred solution of 288-10 (1 g, 3.741 mmol, 1 equiv) in DCE (30 mL, 378979 mmol, 101.31 equiv) was added 5-bromo-3-(trifluoromethyl)picolinaldehyde (1.14 g, 4.488 mmol, 1.20 equiv) at room temperature. The resulting mixture was stirred overnight at room temperature. To the above mixture was added NaBH(OAc)3 (2.38 g, 11,223 mmol, 3 equiv) at room temperature. The resulting mixture was stirred for additional 4 h at room temperature. The reaction was quenched with saturated NaHCO3 (aq.) (100 mL) at room temperature. The aqueous layer was extracted with DCM (3×100 mL). The resulting mixture was concentrated under vacuum. The residue was purified by Prep-TLC (DC M/MeOH 20:1) to afford 557-1 (1 g, 51.84%) as a white solid.

### 2. Synthesis of 557-2

To a stirred solution of 557-1 (1 g, 1.979 mmol, 1 equiv) and Pyridine (1.57 g, 19.790 mmol, 10 equiv) in DCM (30 mL) was added Triphosgene (0.23 g, 0,792 mmol, 0.4 equiv) at 0° C. The resulting mixture was stirred for 10 min at room temperature. The reaction was quenched with saturated NaHCO₃ (aq.) (30 mL) at room temperature. The aqueous layer was extracted with DCM (2×50 mL). The resulting mixture was concentrated under vacuum. The residue was purified by Prep-TLC (DCM/MeOH 20:1) to afford 557-2 (850 mg, 76.80%) as an orange solid,

### 3. Synthesis of 557-3

To a solution of 557-2 (800 ng, 1.506 mmol, 1 equiv) and TMEDA (349.93 mg, 3.012 mmol, 2 equiv) in 1,4-dioxane (25 mL) were added bis(adamantan-1-yl)(butyl)phosphane (107.97 mg, 0.301 mmol, 0.2 equiv) and Pd(OAc)₂ (33.80 mg, 0.151 mmol, 0.1 equiv) in a pressure tank. The mixture was purged with nitrogen for 3 min and then was pressurized to 15 atm with CO/1-=1:1 at 85° C. for overnight. The reaction mixture was cooled to room temperature and concentrated under vacuum. The residue was purified by silica gel column chromatography, eluted with DCM/MeOH (20:1) to afford 557-3 (540 mg, 67.18%) as a yellow solid.

### 4. Synthesis of 557-4

To a stirred solution of 557-3 (520 mg, 1,082 mmol, 1 equiv) and 5-azaspiro[2.3]hexane hydrochloride (258.87 mg, 2.164 mmol, 2 equiv) in DCE (8 mL) were added TEA (219.05 mg, 2.164 mmol, 2 equiv) at room temperature. The resulting mixture was stirred for 2 h at room temperature. To the above mixture was added NaBH(OAc)₃ (458.77 mg, 2.164 mmol, 2 equiv) at room temperature. The resulting mixture was stirred for additional 3 h at room temperature. The reaction was quenched with saturated NH₄Cl (aq.) (30 mL) at room temperature. The aqueous layer was extracted with DCM (2×30 mL). The resulting mixture was concentrated under vacuum. The residue was purified by Prep-TLC (DCM/MeOH 15:1) to afford 557-4 (150 mg, 24.30%) as a yellow solid.

### 5. Synthesis of 557

The 557-4 (150 mg) was purified by Prep-Chiral-HPLC with the following conditions (Column: CHIRAL ART Amylose-SA, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 8 min; Wave Length: 220/254 nm; RT1 (min): 3.91; RT2 (min): 6.07; the second peak is product) to afford 557 (39.2 mg, 26.08%) as a yellow solid.

LC-MS-557: (ES, m/z): [M+H]⁺ 548

H-NMR-557: (400 MHz, DMSO-d6, δ ppm): 0.51 (s, 411), 1.67-1.84 (m, 5H), 2.03-2.08 (m, 1H), 3.21-3.31 (m, 5H), 3.45 (s, 2H), 3.47 (s, 3H), 4.37-4.40 (d, 1H), 7.02 (s, 1H), 7.51 (s, 1H), 7.67-7.69 (m, 2H), 8.23-8.24 (d, 2H), 8.36 (s, 1H).

### Example 549. Synthesis of Compound 558

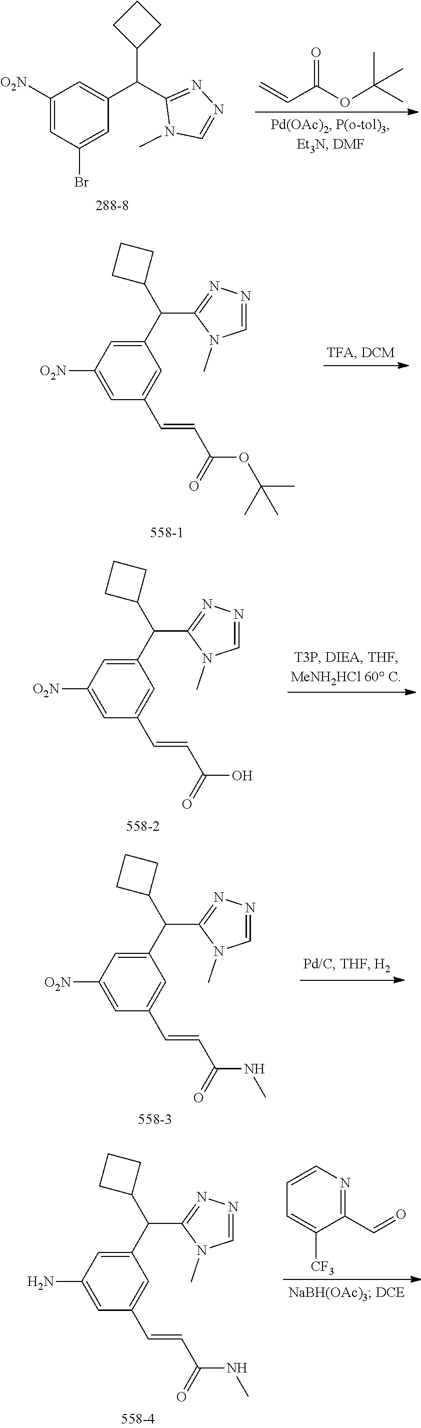

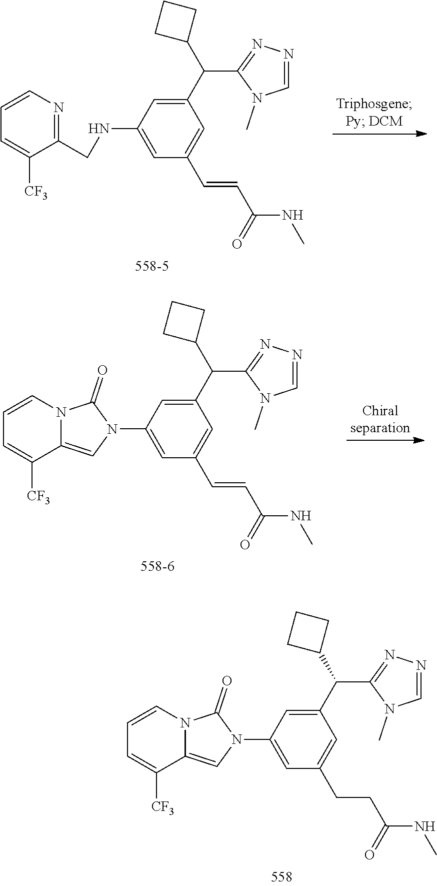 [SMILES: CNC(=O)/C=C/c1cc(NCc2ncccc2C(F)(F)F)cc(C(c2nncn2C)C2CCC2)c1 ; CNC(=O)/C=C/c1cc(C(c2nncn2C)C2CCC2)cc(-n2cc3c(C(F)(F)F)cccn3c2=O)c1 ; CNC(=O)CCc1cc([C@@H](c2nncn2C)C2CCC2)cc(-n2cc3c(C(F)(F)F)cccn3c2=O)c1]

### 1. Synthesis of 558-1

To a stirred solution of 288-8 (2 g, 5.69 mmol, 1.0 equiv) and tert-butyl prop-2-enoate (1.5 g, 11.39 mmol, 20 equiv) in DMF (20 mL) were added Et₃N (2.3 g, 22.78 mmol, 4.0 equiv), Pd(OAc)2 (127 mg, 0.57 mmol, 0.1 equiv) and P(o-tol)₃ (346.66 mg, 1,139 mmol, 0.2 equiv) at room temperature under nitrogen atmosphere. The resulting mixture was stirred for overnight at 100° C. under nitrogen atmosphere. The reaction was quenched by the addition of water (50 mL) at room temperature. The resulting mixture was extracted with EtOAc (3×30 mL). The combined organic layers were washed with brine (3×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 558-1 (2 g, 72%) as an off-white solid,

### 2. Synthesis of 558-2

A solution of 558-1 (2 g, 5.01 mmol, 1.0 equiv) and TFA (5 ml) in DCM (15 mL) was stirred for overnight at room temperature. The mixture was neutralized to pH 8 with NH₃ in MeOH. The resulting mixture was concentrated under reduced pressure. The residue was purified by silica gel column chromatography, eluted with CHCl2/MeOH (10:1) to afford 558-2 (1.5 g, 87%) as an off-white solid.

### 3. Synthesis of 558-3

To a stirred solution of 558-2 (980 mg, 2.86 mmol, 1.0 equiv) and methylamine (88 mg, 2.86 mmol, 1.0 equiv) in THF (10 mL) were added T₃P (2.7 g, 8.59 mmol, 3.0 equiv) and DIEA (1.5 g, 11.45 mmol, 4.0 equiv) at room temperature. The resulting mixture was stirred for 3 h at 60° C. The reaction was quenched by the addition of water (30 ml) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (PE/EA=1:1) to afford 558-3 (400 mg, 39%) as an off-white solid.

### 4. Synthesis of 558-4

To a solution of 558-3 (540 mg, 1.51 mmol, 1.0 equiv) in 20 mL THF was added Pd/C (10%, 100 mg) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure. This resulted in 558-4 (400 mg, 80%) as an off-white solid.

### 5. Synthesis of 558-5

To a stirred solution of 558-4 (380 mg, 1.16 mmol, 1.0 equiv) and 3-(trifluoromethyl) pyridine-2-carbaldehyde (203 mg, 1.16 mmol, 1.0 equiv) in DCE (4 mL) was added NaBH(OAc)3 (491 mg, 2.32 mmol, 2.0 equiv) at room temperature. The resulting mixture was stirred for overnight at room temperature. The reaction was quenched by the addition of water (20 mL) at room temperature. The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The residue was purified by Prep-TLC (CH₂Cl₂/MeOH=10:1) to afford 558-5 (320 mg, 57%) as an off-white solid.

### 6. Synthesis of 558-6

To a stirred solution of 558-5 (320 mg, 0.65 mmol, 1.0 equiv) and pyridine (312 mg, 3.94 mmol, 6.0 equiv) in DCM (3 mL) was added triphosgene (68 mg, 0.23 mmol, 0.35 equiv) at 0° C. The resulting mixture was stirred for 1 h at 0° C. The reaction was quenched by the addition of sat. NaHCO₃ (aq.) (30 mL) at room temperature. The resulting mixture was extracted with CH2Cl₂ (3×20 mL). The combined organic layers were washed with water (3×20 mL), dried over anhydrous Na2SO₄. After filtration, the filtrate was concentrated under reduced pressure. The residue was purified by Prep-TLC (CH2Cl₂/MeOH=10:1) to afford 558-6 (40 mg, 12%) as a yellow solid.

### 7. Synthesis of 558

The 558-6 (280 mg) was purified by chiral separation with the following conditions (Prep-HPLC-037): Column: Lux 5 um Cellulose-2, 2.12*25 cm, 5 um; Mobile Phase A: Hex (0.5% 2M NH₃-MeOH)-HPLC, Mobile Phase B: EtOH-HPLC; Flow rate: 20 mL/min; Gradient: 50% B to 50% B in 20 min; Wave Length: 220/254 am; RT1 (min): 12.61; RT2 (min): 16.64; the first peak was the product. Sample Solvent: EtOH-HPLC; Injection Volume: 0.6 mL; Number of Runs: 11. This resulted in 558 (92 mg, 33%) as a yellow solid.

LC-MS-558: (ES, m/z): [M+H]⁺ 513

H-NMR-558: (400 MHz, DMSO-d6, δ ppm): 1.73-1.78 (m, 1H), 1.79-1.84 (m, 4H), 2.05-2.09 (m, 1H), 2.33-2.37 (t, 2H), 2.54 (s, 3H), 2.80-2.84 (t, 2H), 3.17-3.25 (m, 1H), 3.41 (s, 3H), 4.18-4.21 (m, 1H), 6.27-6.30 (t, 1H), 7.02 (s, 1H), 7.09-7.10 (d, 1H), 7.30 (s, 1H), 7.51 (s, 1H), 7.62 (s, 1H), 7.76-7.78 (d, 1H), 8.32 (s, 1H).

### Example 550. Synthesis of Compound 559

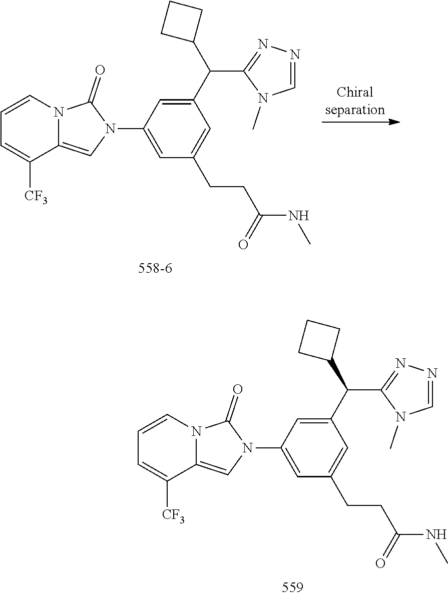 [SMILES: CNC(=O)CCc1cc(C(c2nncn2C)C2CCC2)cc(-n2cc3c(C(F)(F)F)cccn3c2=O)c1 ; CNC(=O)CCc1cc([C@H](c2nncn2C)C2CCC2)cc(-n2cc3c(C(F)(F)F)cccn3c2=O)c1]

### 1. Synthesis of 559

The 558-6 (280 mg) was purified by chiral separation with the following conditions (Prep-HPLC-037): Column: Lux 5 um Cellulose-2, 2.12*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 20 min; Wave Length: 220/254 am; RT1 (min): 12.61; RT2 (min): 16.64; The second peak was the product. Sample Solvent: EtOH-HPLC; Injection Volume: 0.6 mL; Number of Runs: 11. This resulted in 559 (94.7 mg, 33.89%) as a yellow solid.

LC-MS-559: (ES, m/z): [M+H]⁺ 513

H-NMR-559: (400 MHz, DMSO-d6, δ ppm): 1.73-1.84 (m, 5H), 2.05-2.09 (m, 1H), 2.33-2.37 (t, 2H), 2.54 (s, 3H), 2.80-2.84 (t, 2H), 3.17-3.25 (m, 1H), 3.41 (s, 3H), 4.18-4.21 (m, 1H), 6.27-6.30 (t, 1H), 7.02 (s, 1H), 7.09-7.10 (d, 1H), 7.30 (s, 1H), 7.51 (s, 1H), 7.62 (s, 1H), 7.76-7.78 (d, 1H), 8.32 (s, 1H).

### Example 551. Synthesis of Compound 560

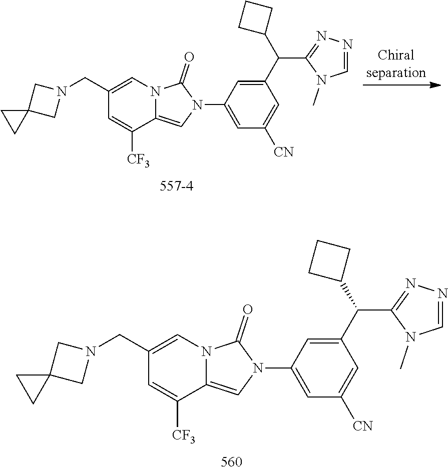 [SMILES: Cn1cnnc1C(c1cc(C#N)cc(-n2cc3c(C(F)(F)F)cc(CN4CC5(CC5)C4)cn3c2=O)c1)C1CCC1 ; Cn1cnnc1[C@H](c1cc(C#N)cc(-n2cc3c(C(F)(F)F)cc(CN4CC5(CC5)C4)cn3c2=O)c1)C1CCC1]

### 1. Synthesis of 560

The 557-4 (150 mg) was purified by Prep-Chiral-HPLC with the following conditions (Column: CHIRAL ART Amylose-SA, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 8 min; Wave Length: 220/254 nm; RT1 (min): 3.91; RT2 (min): 6.07; the first peak is product) to afford 560 (44.2 mg, 29.08%) as a yellow solid.

LC-MS-560: (ES, m/z): [M+H]⁺ 548

H-NMR-560: (400 MHz, DMSO-d6, δ ppm): 0.51 (s, 4H), 1.67-1.84 (m, 5H), 2.03-2.08 (m, 1H), 3.21-3.31 (m, 5H), 3.45 (s, 2H), 3.47 (s, 3H), 4.37-4.40 (d, 1H), 7.02 (s, 1H), 7.51 (s, 1H), 7.67-7.69 (m, 2H), 8.23-8.24 (d, 2H), 8.36 (s, 1H).

### Example 552. Synthesis of Compound 561

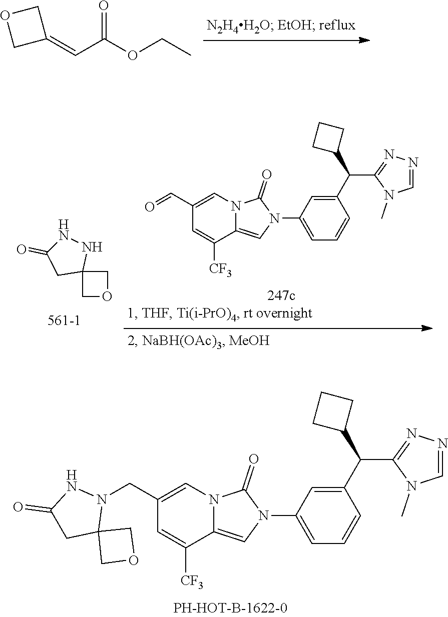

### 1. Synthesis of 561-1

To a stirred solution of ethyl 2-(oxetan-3-ylidene)acetate (3 g, 21.104 mmol, 1 equiv) inEtOH (50 mL) was added hydrazine hydrate (98%) (1.58 g, 31.656 mmol, 1.5 equiv). The resulting mixture was stirred for overnight at 80° C. The resulting mixture was concentrated under reduced pressure. The residue was purified by silica gel column chromatography, eluted with CH2Cl2/MeOH (10:1) to afford 561-1 (14 g, 51.77%) as a white solid.

### 2. Synthesis of 561

To a stirred solution of 247c (1 g, 2,196 mmol, 1 equiv) and 561-1 (0.56 g, 4.392 mmol, 2 equiv) in THF was added Ti(Oi-Pr)₄ (1.25 g, 4.392 mmol, 2 equiv). The resulting mixture was stirred for overnight at room temperature under nitrogen atmosphere. The resulting mixture was concentrated under reduced pressure. The residue was dissolved in MeOH (20 mL). To the above mixture was added NaBH(OAc)₃ (1.40 g, 6,588 mmol, 3 equiv). The resulting mixture was stirred for additional 3 h at room temperature. The resulting mixture was diluted with water (80 mL). The resulting mixture was extracted with CH₂Cl₂ (3×100 mL). The combined organic layer: were concentrated under reduced pressure. The residue was purified by Prep-TLC (CH₂Cl₂/MeOH 20:1) to afford −561 (79.8 mg, 6.40%) as a yellow solid.

LCMS: (ES, m/z): [M+H]⁺ 506 NMR: (400 MHz, DMSO, ppm): δ 1.71-1.85 (m, 5H), 2.07-2.10 (m, 1H), 2.82 (s, 2H), 3.21-3.25 (m, 1H), 3.44 (s, 3H), 3.66 (s, 2H), 4.26-4.29 (d, 1H), 4.50-4.52 (d, 2H), 4.78-4.80 (d, 2H), 7.11 (s, 1H), 7.20-7.22 (d, 1H), 7.33 (s, 1H), 7.43-7.47 (m, 3H), 8.34 (s, 1H), 9.43 (s, 1H).

### Example 553. Synthesis of Compound 562

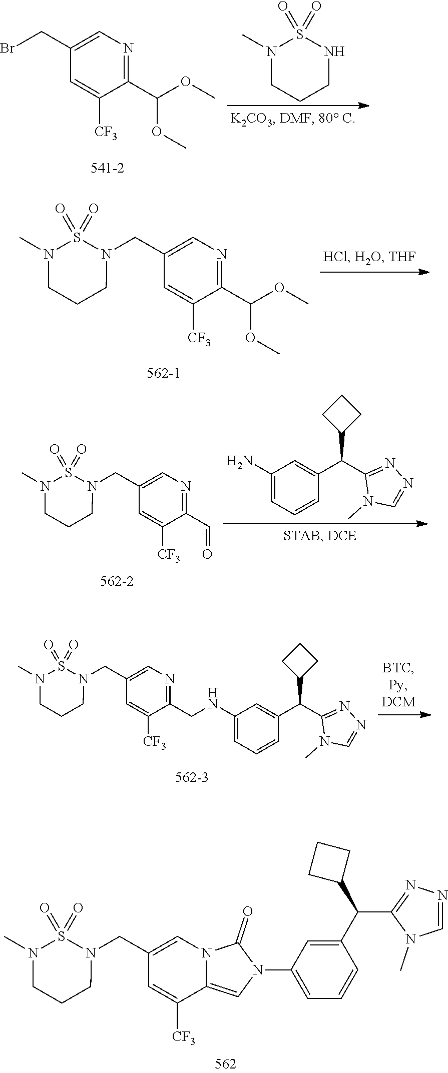

### 1. Synthesis of 562-1

Into a 40 mL vial were added 541-2 (1.3 g, 1.757 mmol, 1 equiv), 2-methyl-1lambda6,2,6-thiadiazinane-1,1-dione (0.93 g, 6.209 mmol, 1.5 equiv), K₂CO₃ (1.14 g, 8.278 mmol, 2 equiv) and DMF (15 mL) at room temperature. The resulting mixture was stirred for overnight at 80° C. The resulting mixture was diluted with water (100 mL). The aqueous layer was extracted with EtOAc (3×40 mL). The resulting mixture was concentrated under vacuum. The residue was purified by Prep-TLC (D)CM/MeOH=50:1) to afford 562-1 (800 mg, 47.39%) as yellow oil.

### 2. Synthesis of 562-2

Into a 40 mL vial were added 562-1 (770 mg, 2.008 mmol, 1 equiv) 1M HC (7 mL) and H2O (2 mL) at room temperature. The resulting mixture was stirred for 2 h at 80° C. The residue was basified to pH 8 with sat. NH₄HCO₃ (aq.) (100 mL). The aqueous layer was extracted with EtOAc (3×30 mL). The resulting mixture was concentrated under vacuum. The residue was purified by Prep-TLC (DCM/MeOH=60:1) to afford 562-2 (600 mg, 84.14%) as a yellow oil.

### 3. Synthesis of 562-3

Into a 50 ml, round-bottom flask were added 608-2 (580 mg, 1.719 mmol 1 equiv), 244b (625.00 mg, 2.579 mmol, 1.5 equiv), STAB (728.83 mg, 3,438 mmol, 2 equiv) and DCE (10 mL) at room temperature. The resulting mixture was stirred for overnight at room temperature. The reaction was quenched with sat. NH4Cl (aq.) (50 mL) at room temperature. The aqueous layer was extracted with EtOAc (3×20 mL). The resulting mixture was concentrated under vacuum. The residue was purified by Prep-TLC (DCM/MeOH=20:1) to afford 562-3 (600 mg, 59.43%) as a yellow oil.

### 4. Synthesis of 562

Into a 100 ml round-bottom flask were added 562-3 (500 mg, 0.887 mmol, 1 equiv), pyridine (561.35 mg, 7.096 mmol, 8 equiv) and DCM (20 mL) at room temperature. To the above mixture was added triphosgene (131.61 mg, 0.444 mmol, 0.5 equiv) at room temperature. The resulting mixture was stirred for 3 min at room temperature. The reaction was quenched with sat. NaHCO3 (aq.) (50 mL) at room temperature. The aqueous layer was extracted with EtOAc (3×20 mL). The resulting mixture was concentrated under vacuum. The crude product (500 mg) was purified by Prep-HPLC with the following conditions (Column: X Bridge Prep OBD C18 Column, 30*150 mm, 5 μm; Mobile Phase A: water (10 mmol/L NH4HCO₃), Mobile Phase B: ACN; Flow rate: 60 mL/min; Gradient: 34% B to 54% B in 8 min; Wave Length: 254 nm; RT1 (min): 7.52) to afford 562 (206.7 tug, 39.48%) as a yellow solid.

LC-MS-562: (ES, m/z): [M+H]⁺ 590

H-NMR-562: 1H NMR (400 MHz, DMSO-d6 ppm) δ 1.82-1.90 (m, 1H), δ 1.91-1.99 (m, 6H), δ 2.24-2.29 (m, 1H), δ 2.83 (s, 3H), δ 3.27-3.29 (m, 1H), δ 3.39-3.45 (m, 4H), δ 3.56 (s, 3H), δ 4.11 (s, 2H), δ 4.28-4.31 (d, 1H), δ 7.03 (s, 1H), δ 7.15 (s, 1H), δ 7.26-7.28 (d, 1H), δ 7.48-7.52 (t, 1H), δ 7.64-7.67 (m, 2H), δ 7.78 (s, 1H), δ 8.38 (s, 1H).

### Example 554. Synthesis of Compound 563

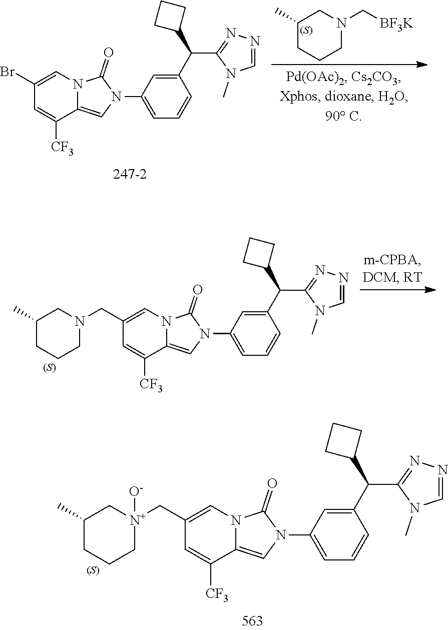

### 1. Synthesis of 563-1

Into a 40 ml sealed tube were added 247-2 (1 g, 1.975 mmol, 1 equiv) and dioxane (8 mL) and H₂O (2 mL) at room temperature. To the above mixture was added (3S)-3-methyl-1-[(trifluoro-lambda4-boranyl)methyl]piperidine potassium (649.09 mg, 2.963 mmol, 1.5 equiv), Xphos (188.31 mg, 0.395 mmol, 0.2 equiv), Cs₂CO₃ (1.93 g, 5.925 mmol, 3 equiv) and Pd(OAC)₂ (22.17 mg, 0.099 mmol, 0.05 equiv) at room temperature. The resulting mixture was stirred for overnight at 90° C. under nitrogen atmosphere. The reaction was quenched with water (10 mL) at room temperature. The resulting mixture was extracted with CH₂Cl₂ (3×10 mL). The combined organic layers were concentrated under reduced pressure. The residue was purified by Prep-TLC (CH₂Cl₂/MeOH 15:1) to afford 563-1 (800 mg, 71.44%) as a light yellow oil.

### 2. Synthesis of 563

Into a 40 mL sealed tube were added 563-1 (500 mg, 0.928 mmol, 1 equiv) and DCM (10 mL) at 0° C. To the above mixture was added m-CPBA (400.47 mg, 2.320 mmol, 2.5 equiv) at 0° C. The resulting mixture was stirred for additional 1 h at 0° C. The mixture was basified to pH 7 with saturated NaHCO₃ (aq.) (10 mL). The resulting mixture was extracted with CH₂Cl₂ (3×10 mL). The combined organic layers were concentrated under reduced pressure. The crude product (300 mg) was purified by Prep-HPLC with the following conditions (Column: Xselect CSH C18 OBD Column 30*150 mm 5 μm, n; Mobile Phase A: Water (0.1% FA), Mobile Phase B: MeOH-HPLC; Flow rate: 60 mL/min; Gradient: 40% B to 55% B in 7 min; Wave Length: 254; 220 nm; RT1 (min): 5.98, 6.05 (min).) to afford 563 (100.4 mg, 17.54%) as a light yellow solid.

LCMS 137-563: (ES, m/z): [M+H]⁺ 555

H-NMR18-563: (300 MHz, DMSO-d6, ppm): 50.85-0.91 (m, 3H), δ 0.99-1.02 (m, 1H), δ 1.64-1.80 (m, 7H), δ 2.01-2.17 (m, 3H), δ 2.89-2.95 (m, 1H), δ 3.19-3.21 (m, 2H), δ 3.31-3.33 (m, 2H), δ 3.33-3.43 (m, 2H), δ 3.63 (s, 1H), δ 4.25-4.28 (m, 1H), δ 4.28-4.49 (m, 2H), δ 7.20-7.22 (m, 1H), δ 7.35-7.42 (m, 2H), δ 7.44-7.46 (m, 1H), δ 7.67-7.73 (m, 2H), δ 8.02-8.25 m, 1H), δ 8.25-8.32 (s, 1H), δ 8.32-8.33 (s, 1H).

### Example 555. Synthesis of Compound 564

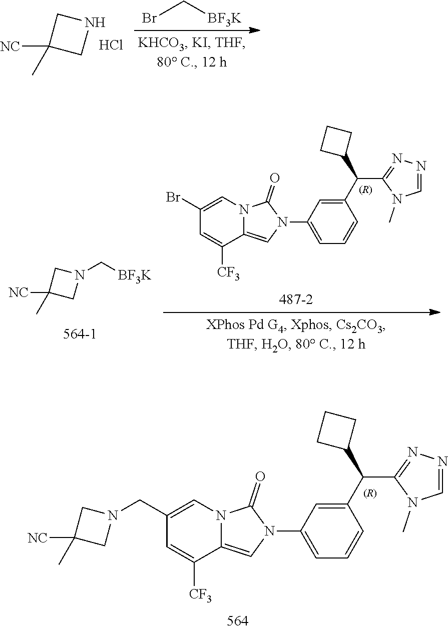

### 1. Synthesis of 564-1

A mixture of potassium;bromomethyl(trifluoro)boranuide (757 mg, 3.77 mmol, 1 equiv), 3-methylazetidine-3-carbonitrile;hydrochloride (500 mg, 3.77 mmol, 1 equiv), KI (62.6 mg, 377 umol, 0.1 equiv), KHCO₃ (755 mg, 7.54 mmol, 2 equiv) in THF (10 mL) was degassed and purged with nitrogen atmosphere for 3 times, and then the mixture was stirred at 80° C. for 12 h under nitrogen atmosphere. The residue was triturated with acetone (20 mL) at 40° C. for 1 hr. Then the mixture was filtered and the filtrate was concentrated in vacuum to afford 564-1 (510 mg, crude) as a yellow oil.

### 2. Synthesis of 564

To a solution of 487-2 (300 mg, 592.51 umol, 1 equiv) and 564-1 (512 mg, 2.37 mmol, 4 equiv) in THF (4 mL) and H₂O (1 mL) was added XPhos (28.2 mg, 59.2 umol, 0.1 equiv) and Cs₂CO₃ (579.16 mg, 1.78 mmol, 3 equiv), dicyclohexyl-[2-(2,4,6-triisopropylphenyl)phenyl]phosphane; methanesulfonate;[22-(methylamino)phenyl]phenyl]palladium(1+) (50.9 mg, 59.2 umol, 0.1 equiv). The mixture was stirred at 80° C. for 12 P under nitrogen atmosphere. The reaction mixture was diluted with water 20 mL and extracted with EtOAc (20 mL×3). The combined organic layers were dried over anhydrous Na2SO4, filtered and concentrated under reduced pressure to give a residue. The residue was purified by flash silica gel chromatography eluted with DCM/MeOH (10:1) to give crude product. The crude product (230 mg) was purified by Prep-HPLC with the following conditions (Column: Waters Xbridge Prep OBD C18 150*40 mm*10 um; Mobile Phase A: Water (10 mmol/L. NH₄CO₃), Mobile Phase B: ACN; Flow rate: 50 mL/min; Gradient: 35% B to 65% B in 8 min; Wave Length: 220 um; RT1 (min): 7.5) to afford 564 (40 mg, 12%) as a yellow solid.

LCMS-564: (ES, m/z): [M+H]⁺ 536.3.

¹H-NMR-564: (400 MHz, ACETONITRILE-d₃) δ 8.08-8.06 (m, 1H), 7.69-7.61 (m, 2H), 7.56 (s, 1H), 7.45 (t, J=8.0 Hz, 1H), 7.25 (d, J=7.6 Hz, 1H), 7.00 (s, 1H), 6.93 (s, 1H), 4.16 (d, J=10.8 Hz, 1H), 3.56 (d, J=7.6 Hz, 2H), 3.44-3.40 (m, 5H), 3.37-3.26 (m, 1H), 3.21 (d, J=7.6 Hz, 2H), 2.14-2.11 (m, 1H), 1.91-1.83 (m, 4H), 1.80-1.74 (m, 1H), 1.60 (s, 3H).

### Example 556. Synthesis of Compound 565_P1

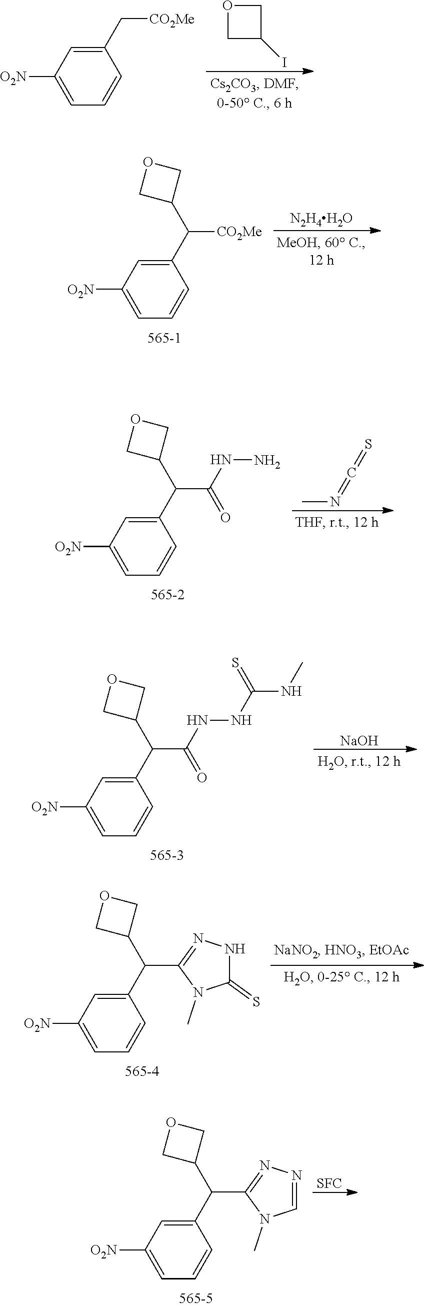

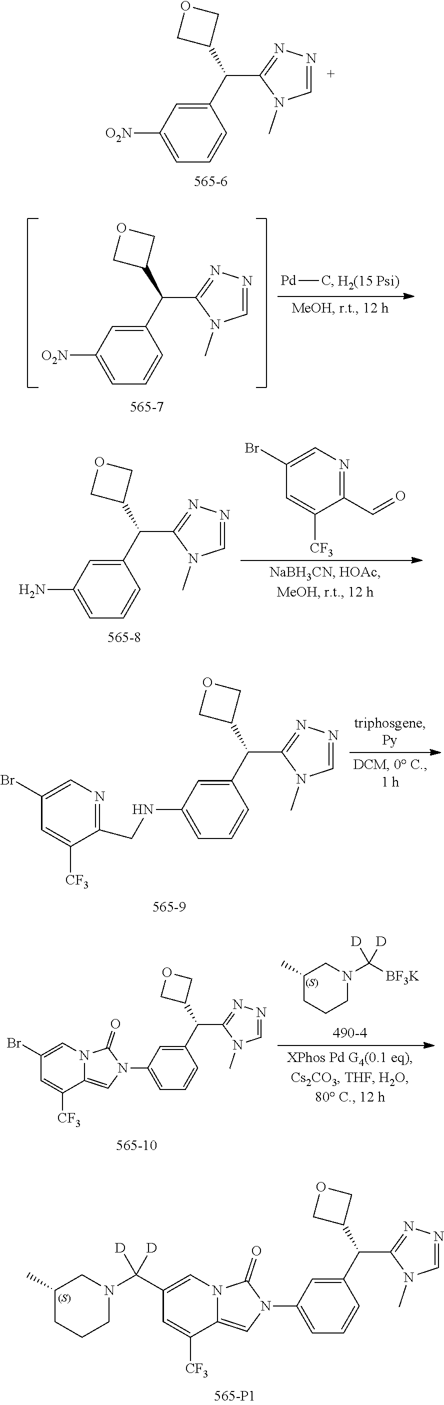

### 1. Synthesis of 565-1

To a solution of methyl 2-(3-nitrophenyl)acetate (25 g, 128.09 mmol, 1 eq) in DMF (300 mL) was added Cs₂CO₃ (208.67 g, 640.46 mmol, 5 equiv). Then 3-iodooxetane (70.70 g, 384.28 mmol, 3 equiv) was added to the reaction mixture at 0° C. Then the mixture was stirred for 3 h at 25° C. Then the mixture was heated to 50° C. and stirred for 3 h at 50° C. under nitrogen atmosphere. The reaction mixture was diluted with H2O (400 ml) and extracted with EtOAc 1800 mL (600 mL×3). The combined organic layers were washed with brine 1500 mL (500 mL×3), dried over Na₂SO₄, filtered and concentrated under reduced pressure to give a residue. The residue was purified by column chromatography (SiO₂, Petroleum ether/Ethyl acetate=5:1 to 1:10) to afford 565-1 (10.9 g, 32%) as a brown solid.

¹H-NMR-565-1: (400 MHz, CHLOROFORM-d) δ 8.21-8.12 (m, 2H), 7.64-7.58 (m, 1H), 7.58-7.51 (m, 1H), 4.96 (dd, J=6.8, 7.6 Hz, 1H), 4.62 (dd, J=6.8, 7.6 Hz, 1H), 4.52 (t, J=6.4 Hz, 1H), 4.26 (t, J=6.4 Hz, 1H), 4.20 (d, J=12 Hz, 1H), 3.70 (s, 4H)

### 2. Synthesis of 565-2

To a solution of 565-1 (4.5 g, 17.91 mmol, 1 equiv) in MeOH (25 mL) was added N₂H₄·H₂O (5 g, 97.88 mmol, 98% purity, 5.46 equiv). The mixture was heated to 45° C. and stirred for 12 h at 45° C. The reaction mixture was concentrated under reduced pressure to give a residue. The residue was diluted with H₂O (30 mL), and extracted with DCM (25 mL×3). The combined organic layers were dried over Na2SO4, filtered and concentrated under reduced pressure to afford 565-2 (3.7 g, crude) as a white solid.

### 3. Synthesis of 565-3

To a solution of 565-2 (3.2 g, 12.74 mmol, 1 equiv) in THF (35 mL) was added isothiocyanatomethane (2.05 g, 28.02 mmol, 1.91 mL, 2.2 equiv). The mixture was stirred at 20° C. for 12 h. The mixture was diluted with H₂O (30 mL). The mixture was concentrated under reduced pressure to remove THF. Then the mixture was filtered to get a filter cake and the filter cake was concentrated in vacuum to afford 565-3 (4.2 g, crude) as a white solid.

### 4. Synthesis of 565-4

To a solution of NaOH (2.47 g, 61.66 mmol, 8 equiv) in H2O (30 mL) was added 565-3 (2.5 g, 7.71 mmol, 1 equiv). The mixture was stirred at 20° C. for 12 h. The mixture was adjusted to pH=2 by HCl (2 M) and filtered. The filter cake was concentrated in vacuum to afford 565-4 (2.7 g, crude) as a white solid.

### 5. Synthesis of 565-5

To a solution of 565-4 (4.2 g, 13.71 mmol, 1 equiv) in DCM (70 mL) was cooled to 0° C. Then a solution of H₂O₂ (6.49 g, 57.24 mmol, 30% purity, 4.17 equiv) in AcOH (6.59 g, 109.68 mmol, 8 equiv) was added to the mixture at 0° C. The result mixture was stirred at 25° C. for 12 h. The solvent was diluted with H2O 30 mL and adjusted pH to 12 by 2N NaOH, and extracted with DCM (90 mL×3). The combined organic layers were dried over Na2SO4, filtered and concentrated under reduced pressure to give a residue. The residue was purified by column chromatography (SiO₂, DCM/MeOH=50/1 to 5/1) to afford 565-5 (1.2 g, 33%) as a yellow solid,

¹H-NMR-565-5: (400 MHz, METHANOL-d₄) δ=8.41 (s, 1H), 8.23-8.11 (m, 2H), 7.74-7.56 (m, 2H), 4.99-4.89 (m, 2H), 4.67-4.55 (m, 2H), 4.41 (t, J=6.4 Hz, 1H), 4.05-3.93 (m, 1H), 3.49 (s, 3H)

### 6. Synthesis of 565-6&7

The 565-5 (3.3 g) was purified by Chiral separation with the following conditions (column: DAICEL CHIRALCEL OD (250 mm*50 mm, 10 um);mobile phase: [0.1% NH3H₂O ETOH]; B %: 45%-45%, 8.5min, Flow rate: 70 mL/min; Wave Length: 220/254 nm; RT1 (min): 4.71; RT2 (mm): 6.03) to afford 565-6 (1.5 g) as a yellow solid and 565-7 (1.5 g) as a yellow solid.

¹H-NMR-565-6: (400 MHz, METHANOL-d₄) δ 8.41 (s, 1H), 8.23-8.13 (m, 2H), 7.75-7.57 (m, 2H), 5.00-4.88 (m, 2H), 4.67-4.54 (m, 2H), 4.41 (t, J=6.4 Hz, 1H), 3.99 (td, J=6.4, 12 Hz, 1H), 3.50 (s, 3H)

¹H-NMR-565-7: (400 MHz, METHANOL-d₄) δ 8.41 (s, 1H), 8.23-8.13 (m, 2H), 7.75-7.57 (m, 2H), 5.00-4.88 (m, 2H), 4.67-4.54 (m, 2H), 4.41 (t, J=6.4 Hz, 1H), 3.99 (td, J=6.4, 11.6 Hz, 1H), 3.50 (s, 3H)

### 7. Synthesis of 565-8

To a solution of 565-6 (500 mg, 1.82 mmol, 1 equiv) in EA (5 mL) was added Pd/C (500 mg, 10% purity, 0.25 equiv) under N₂ atmosphere. The suspension was degassed and purged with H₂ for 3 times. The mixture was stirred under H₂ (15 Psi) at 15° C. for 12 h. The reaction mixture was filtered and concentrated under reduced pressure to afford 565-8 (450 mg, crude) as a yellow oil.

### 8. Synthesis of 565-9

To a solution of 5-bromo-3-(trifluoromethyl)picolinaldehyde (467.89 mg, 1.84 mmol, 1 equiv) in MeOH (10 mL) was added 565-8 (450 mg, 1.84 mmol, 1 equiv), AcOH (110.62 mg, 1.84 mmol, 1 equiv). The mixture was stirred for 30 min at 15° C. Then sodium;cyanoboranuide (231.52 mg, 3.68 mmol, 2 equiv) was added to the reaction mixture and stirred for 11.5 h at 15° C. The reaction mixture was diluted with H₂O (20 mL) and extracted with EtOAc (20 mL×3). The combined organic layers were washed with brine (50 mL), dried over Na2SO4, filtered and concentrated under reduced pressure to give a residue. The residue was purified by column chromatography (SiO₂, Petroleum ether/Ethyl acetate=5:1 to DCM: MeOH 1:3) to afford 565-9 (650 mg, 59.9%) as a brown solid.

¹H-NMR-565-9: (400 MHz, CHLOROFORM-d) δ 8.81 (d, J=1.6 Hz, 1H), 8.11 (d, J=2.0 Hz, 1H), 8.03 (s, 1H), 7.16 (t, J=7.8 Hz, 1H), 6.65-6.56 (m, 2H), 6.51 (s, 1H), 5.27 (s, 1H), 5.08 (t, J=7.2 Hz, 1H), 4.69 (t, J=7.2 Hz, 1H), 4.57-4.46 (m, 3H), 4.39-4.27 (m, 2H), 4.03-3.91 (m, 1H), 3.40-3.32 (m, 3H)

### 9. Synthesis of 565-10

To a solution of 565-9 (300 mg 62202 umol, 1 equiv) in DCM (6 mL) was added pyridine (295.21 mg, 3.73 mmol, 6 equiv), bis(trichloromethyl) carbonate (60.00 mg, 202.19 umol, 0.25 equiv) at 0° C. and stirred for 30 min. The reaction mixture was diluted with Sat.NaHCO₃ (10 mL), extracted with DCM (20 mL×3). The combined organic layers were dried over Na2SO₄, filtered and concentrated in vacuum to get a residue. The residue was purified by column chromatography (SiO₂, Petroleum ether/Ethyl acetate=5:1 to DCM: MeOH=1:1) to afford 565-10 (310 mg, 78.4%) as a yellow solid.

¹H-NMR-565-10: (400 MHz, CHLOROFORM-d) δ 8.07 (s, 1H), 7.87 (s, 1H), 7.63 (s, 1H), 7.57 (d, J=1.2 Hz, 1H), 7.49-7.44 (m, 1H), 7.19 (d, J=8.8 Hz, 1H), 6.89 (s, 1H), 6.79 (s, 1H), 5.06 (d, J=7.2 Hz, 1H), 4.72 (d, J=7.2 Hz, 1H), 4.54-4.47 (m, 2H), 4.34 (d, J=6.0 Hz, 1H), 4.05 (td, J=5.9, 11.2 Hz, 1H), 3.44 (s, 3H)

### 10. Synthesis of 565_P1

A mixture of 490-4 (256.65 mg, 1.16 mmol, 2 equiv), 565-10 (295 mg, 580.38 umol, 1 equiv), dicyclohexyl-[2-(2,4,6-triisopropylphenyl)phenyl]phosphane;methanesulfonate;[2-[2-(methylamino)phenyl]phenyl]palladium(1+) (49.94 ng, 58.04 umol, 0.1 equiv), dicesium;carbonate (567.29 mg, 1.74 mmol, 3 equiv) in THF (6 mL)), H2O (1.5 mL) was degassed and purged with N₂ for 3 times. Then the mixture was stirred at 80° C. for 12 h under N₂ atmosphere. The mixture was concentrated under reduced pressure and diluted with H₂O (15 mL), extracted with DCM (20 mL×3), dried over anhydrous Na2SO₄, filtered and the filtrate was concentrated under reduced pressure to give a residue. The residue was purified by column chromatography (SiO₂, Petroleum ether/Ethyl acetate=10/1 to 0/1) then with (DCM: Methanol=100/1 to 0/1) to afford 565_P1 (0.3 g, crude) as a brown solid. The crude product (0.3 g) was further purified by prep-HPLC with the following conditions (Column: Phenomenex Luna C18 75*30 mm*3 um; mobile phase: [water (FA)-ACN]; B %: 10%-40%, 8mi; Wave Length: 220 nm; RT 1 (main): 6.5) to afford 565_P1 (54.1 mg, 22%) as a yellow solid.

MS-565_P1: (ES, m/z): [M+H]+ 543.3.

¹H-NMR-565_P1: (400 MHz, METHANOL-d₄) δ 8.39 (s, 1H), 8.37 (m, 0.5H), 7.84 (s, 1H), 7.72-7.63 (m, 2H), 7.55-7.51 (t, J=16 Hz, 1H), 7.30-7.28 (d, J=8 Hz, 1H), 7.20 (s, 1H), 7.12 (s, 1H), 4.96-4.92 (t, J=16 Hz, 1H), 4.84 (s, 1H), 4.68-4.61 (m, 2H), 4.43-4.40 (t, J=12 Hz, 1H), 4.03-3.97 (m, 1H), 3.50 (s, 3H), 3.24-3.15 (m, 2H), 2.49-2.43 (t, J=24 Hz, 1H), 2.21-2.15 (t, J=24 Hz, 1H), 1.87-1.75 (m, 3H), 1.71-1.65 (m, 1H), 1.13-1.05 (m, 1H), 0.96-0.95 (d, J=4 Hz, 3H).

### Example 557. Synthesis of Compound 565_P2

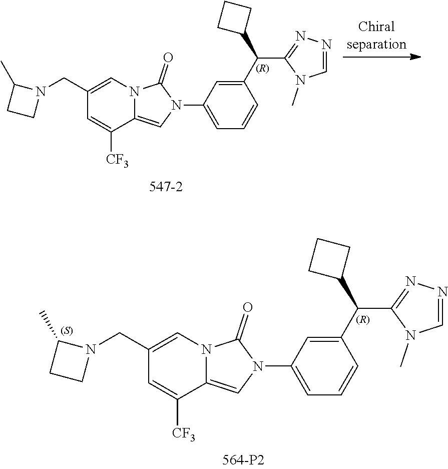

### 1. Synthesis of 565_P2

547-2 (390 mg) was purified by Chiral separation with the following conditions (column: DAICEL CHIRALPAK IC (250 mm*30 mm, 10 um); mobile phase: [MeOH (0.1% IPAm)]; B %: 66%-66%, 30 min. Flow rate: 4 mL/min; Wave Length: 220/254 ran; RT1 (min): 1.691) to afford crude 565 P2. The crude product (100 rug) was purified by Prep-HPLC with the following conditions (column: Phenomenex Luna C18 75*30 mm*3 um; mobile phase: [water (FA)-ACN]; B %: 20%-60%, 8 min, Wave Length: 220 nm; RT1 (min): 5.5 min) to afford 565P2 (50 mg, 12.84%) as a yellow solid.

MS-565_P2: (ES, m/z): [M+H]+ 511.2.

1H-NMR-565_P2: (400 MHz, CHLOROFORM-d) δ 8.04 (s, 1H), 7.79 (s, 1H), 7.62 (s, 1H), 7.52-7.48 (m, 1H), 7.43 (t, J=8.0 Hz, 1H), 7.20-7.18 (m, 1H), 7.12 (s, 1H), 6.77 (s, 1H), 4.09-3.84 (m, 5H), 3.52-3.35 (m, 5H), 2.45-2.27 (m, 3H), 1.98-1.80 (m, 4H), 1.76-1.69 (m, 1H), 1.45 (m, 3H).

### Example 558. Synthesis of Compound 566

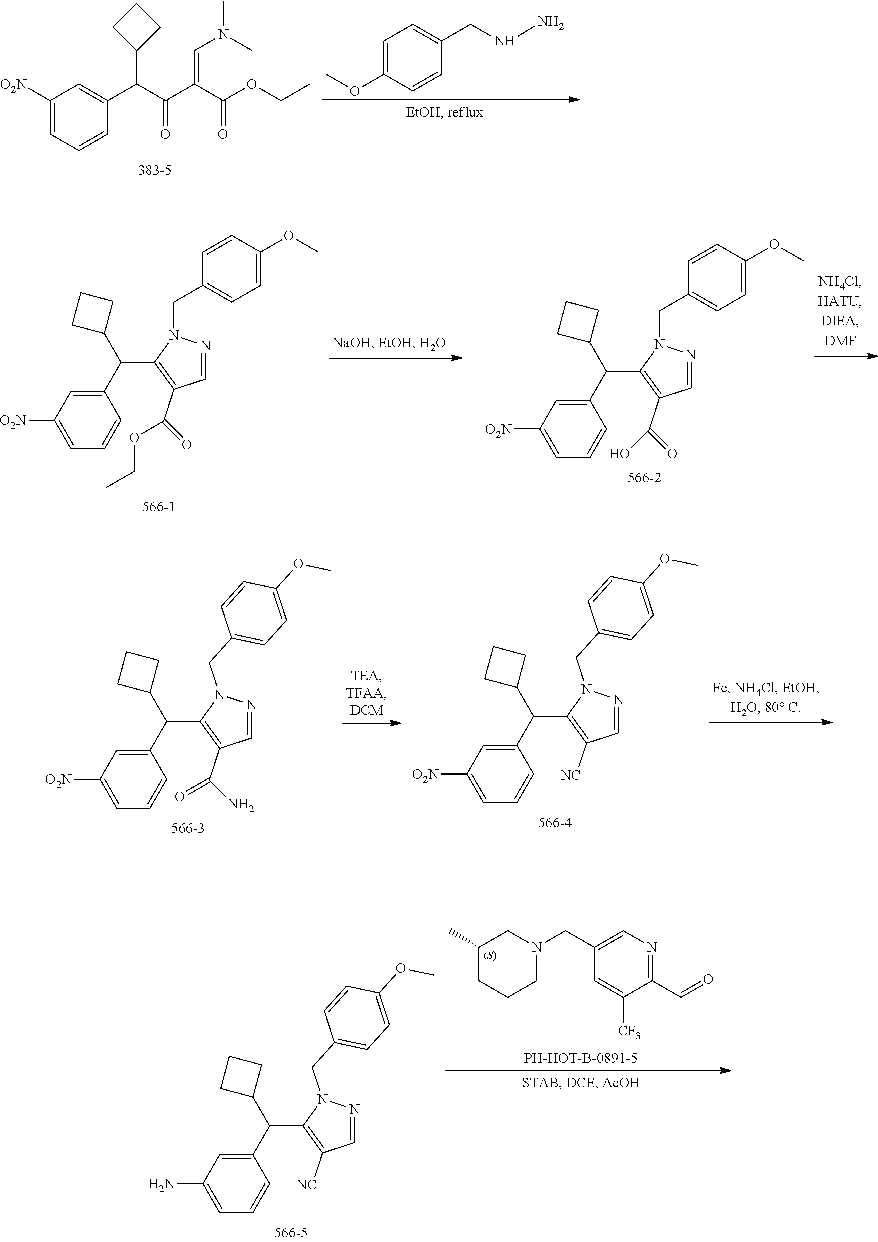

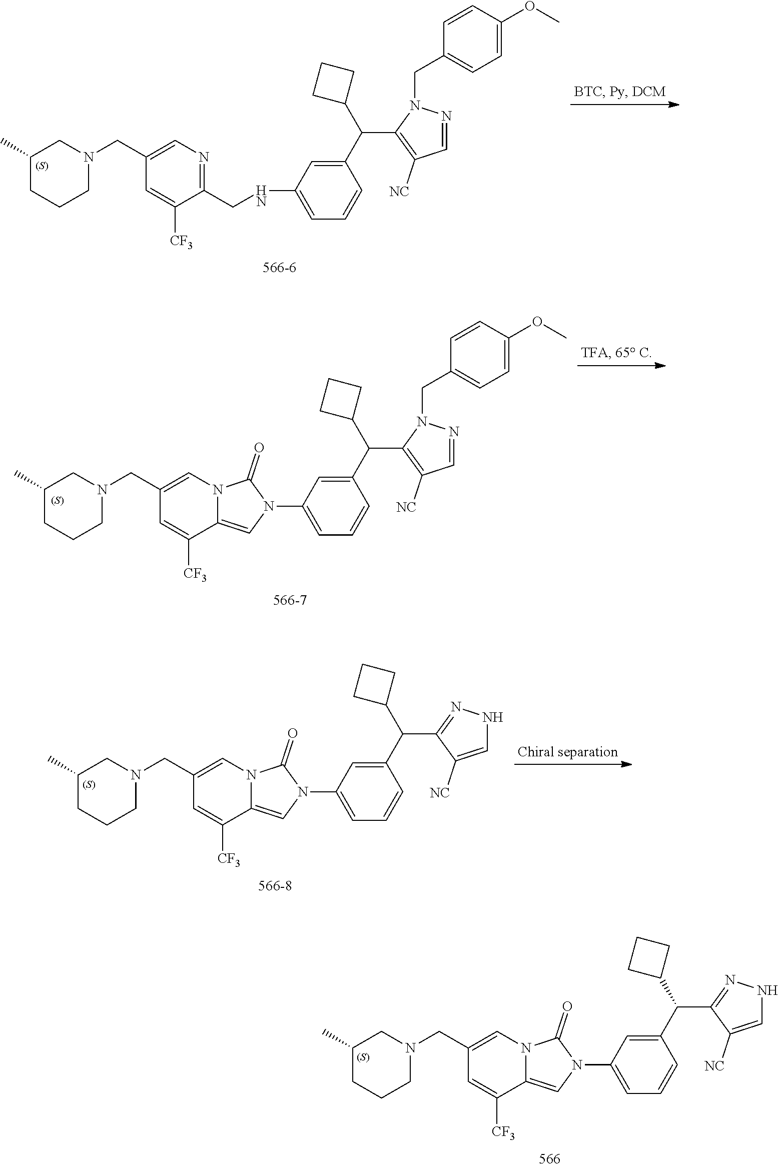

### 1. Synthesis of 566-1

Into 500 mL round-bottom flask were added 383-5 (17 g, 47.169 mmol, 1 equiv), [(4-methoxyphenyl)methyl]hydrazine (8.61 g, 56.603 mmol, 1.2 equiv) and EtOH (200 mL) at room temperature. The resulting mixture was stirred form overnight at 80° C. The resulting mixture was diluted with water (1000 mL). The aqueous layer was extracted with EtOAc (3×400 mL). The resulting mixture was concentrated under vacuum. The reside was purified by silica gel column chromatography, eluted with PE/EA (5:1) to afford 566-1 (15 g, 65.09%) as colorless oil.

### 2. Synthesis of 566-2

Into a 250 mL round-bottom flask were added 566-1 (7 g, 15.573 mmol, 1 equiv), NaOH (1.25 g, 31.146 mmol, 2 equiv), EtOH (70 mL) and H2O (20 mL) at room temperature. The resulting mixture was stirred for overnight at 50° C. The residue was neutralized to pH 7 with 1M HCl (40 mL). The aqueous layer was extracted with EtOAc (3×100 mL). The resulting mixture was concentrated under vacuum. The resulting solid was dried under vacuum. This resulted in 566-2 (6 g, 86.85%) as a white solid.

### 3. Synthesis of 566-3

Into a 250 mL round-bottom flask were added 566-2 (6 g, 14.236 mmol, 1 equiv), NH₄Cl (3.81 g, 71.180 mmol, 5 equiv), HATU (10.83 g, 28.472 mmol, 2 equiv), DIEA (5.52 g, 42.708 mmol, 3 equiv) and DMF (70 ml) at room temperature. The resulting mixture was stirred for overnight at room temperature. The resulting mixture was diluted with water (300 mL). The aqueous layer was extracted with EtOAc (3×150 mL). The resulting mixture was concentrated under vacuum. The residue was purified by silica gel column chromatography, eluted with DCM/MeOH (80:1) to afford 566-3 (5 g, 77.68%) as a white oil.

### 4. Synthesis of 566-4

Into a 250 mL round-bottom flask were added 566-3 (3 g, 7.135 mmol, 1 equiv), DCM (50 mL) and TEA (3.61 g, 35.675 mmol, 5 equiv) at room temperature. To the above mixture was added TFA A (4.50 g, 21,405 mmol, 3 equiv) dropwise at room temperature. The resulting mixture was stirred for 5 h at room temperature. The resulting mixture was diluted with water (300 m L). The aqueous layer was extracted with EtOAc (3×150 ML). The resulting mixture was concentrated under vacuum. The residue was purified by silica gel column chromatography, eluted with DCM/MeOH (100:1) to afford 566-4 (2 g, 64.08%) as a white oil.

### 5. Synthesis of 566-5

Into a 100 ml, round-bottom flask were added 566-4 (2 g 4.970 mmol, 1 equiv), Fe (1.39 g, 24.850 mmol, 5 equiv), NH₄Cl (2.66 g, 49.700 mmol, 10 equiv), EtOH (20 ML) and 1-1H2 (5 mL) at room temperature. The resulting mixture was stirred for overnight at 80° C. The resulting mixture was filtered; the filter cake was washed with DCM (3×30 mL). The filtrate was concentrated under reduced pressure. The residue was purified by Prep-TLC (DCM/MeOH=15:1) to afford 566-5 (1.2 g, 60.29%) as a white oil.

### 6. Synthesis of 566-6

Into a 100 ml, round-bottom flask were added 566-5 (1.2 g, 3.222 mmol, 1 equiv), 1-2 (1.11 g, 3.866 mmol, 1.2 equiv), STAB (1.37 g, 6.444 mmol, 2 equiv) and DCE (20 mL) at room temperature. The resulting mixture was stirred for overnight at room temperature. The reaction was quenched with sat, NH₄Cl aq.) (100 mL) at room temperature. The aqueous layer was extracted with EtOAc (3×40 mL). The resulting mixture was concentrated under vacuum. The residue was purified by Prep-TLC (D)CM/MeOH=20:1) to afford 566-6 (1.2 g, 53.31%) as a colorless oil.

### 7. Synthesis of 566-7

Into a 100 mL round-bottom flask were added 566-6 (1.2 g, 1.867 mmol, 1 equiv), pyridine (1.18 g, 14.936 mmol, 8 equiv) and DCM (30 mL) at room temperature. To the above mixture was added triphosgene (0.28 g, 0.933 mmol, 0.5 equiv) at room temperature. The resulting mixture was stirred for 3 min at room temperature. The reaction was quenched with sat. NaHCO₃ (aq.) (100 mi) at room temperature. The aqueous layer was extracted with EtOAc (3×40 mL). The resulting mixture was concentrated under vacuum. The residue was purified by Prep-TLC (DCM/MeOH=20:1) to afford 566-7 (700 mg, 52.14%) as a yellow solid.

### 8. Synthesis of 566-8

Into a 50 mL round-bottom flask were added 566-7 (660 mg, 0.987 mmol, 1 equiv) and TFA (10 mL) at room temperature. The resulting mixture was stirred for overnight at 65° C. The resulting mixture was diluted with water (100 mL). The aqueous layer was extracted with EtOAc (3×40 mL). The resulting mixture was concentrated under vacuum. The crude product (440 mg) was purified by Prep-HPLC with the following conditions (Column: X Bridge Prep OBD C18 Column, 30*150 mm, 5 μm; Mobile Phase A: water (10 mmol/L NH₄HCO₃), Mobile Phase B: ACN; Flow rate: 60 mL/min; Gradient: 60% B to 85% B in 8 min; Wave Length: 254 nm; RT1 (min): 7.77) to afford 566-8 (140 mg, 25.60%) as a yellow solid.

### 9. Synthesis of 566

The 566-8 (140 mg, 0.255 mmol, 1 equiv) was purified by chiral separation with the following conditions (Column: CHIRAL ART Amylose-SA, 2*25 cm, 5 μm; Mobile Phase A: Hex (0.5% 2M NH₃-MeOH)-HPLC, Mobile Phase B: EtOH: HeX=1:1; Flow rate: 20 mL/min; Gradient: 10% B to 10% B in 21 min; Wave Length: 220/254 nm; RT1 (min): 10.04; RT2 (min): 12.22; The first peak was the product. Sample Solvent: EtOH: HeX=1:1; Injection Volume: 0.4 mL; Number of Runs: 8) to afford 566 (52.8 mg, 37.64%) as a yellow solid.

LC-MS-566: (ES, m/z): [M+H]⁺ 549

H-NMR-566: 1H NMR (400 MHz, DMSO-d6 ppm) 50.79-0.94 (m, 4H), 51.44-1.47 (m, 1H), 61.58-1.70 (m, 5H), 51.76-1.88 (m, 5H), 61.90-2.01 (m, 1H), 62.76 (s, 2H), 63.26 (s, 3H), 54.21-4.24 (d, 1H), 67.01 (s, 1H), 67.25-7.27 (d, 1H), 67.31 (s, 1H), 67.43-7.47 (t, 1H), 67.67-7.72 (m, 2H), 67.77 (s, 1H), 68.31 (s, 1H), 613.59 (s, 1H).

### Example 559. Synthesis of Compound 567

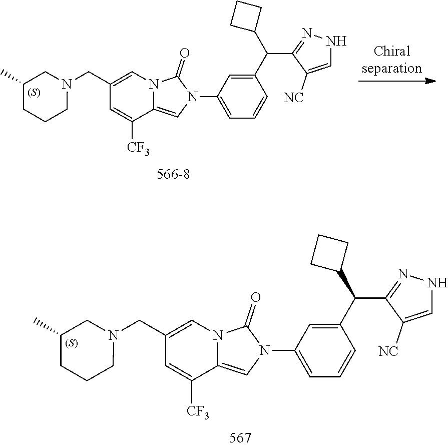

### 1. Synthesis of 567

The 566-8 (140 mg, 0,255 mmol, 1 equiv) was purified by chiral separation with the following conditions (Column: CHIRAL ART Amylose-SA, 2*25 cm, 5 μm; Mobile Phase A: Hex (0.5% 2M NH₃-MeOH)-HPLC, Mobile Phase B: EtOH: HeX=1:1; Flow rate: 20 mL/min; Gradient: 10% B to 10% B in 21 min; Wave Length: 220/254 nm; RT1 (min): 10.04; RT2 (min): 12.22; The second peak was the product. Sample Solvent: EtOH: HeX=1:1; Injection Volume: 0.4 mL; Number of Runs: 8) to afford 567 (56.0 mg, 39.64%) as a yellow solid.

LC-MS-567: (ES, m/z): [M+H]⁺ 549

H-NMR-567: 1H NMR (400 MHz, DMSO-d6 ppm) 60.79-0.94 (m, 4H), 61.44-1.47 (m, 1H), 61.58-1.70 (m, 5H), 61.76-1.88 (m, 5H), 61.90-2.01 (m, 1H), 62.76 (s, 2H), 63.26 (s, 3H), 64.21-4.24 (d, 1H), 67.01 (s, 1H), 67.25-7.27 (d, 1H), 67.31 (s, 1H), 67.43-7.47 (t, 1H), 67.67-7.72 (m, 2H), 67.77 (s, 1H), 68.31 (s, 1H), 613.58 (s, 1H).

### Example 560. Synthesis of Compound 568

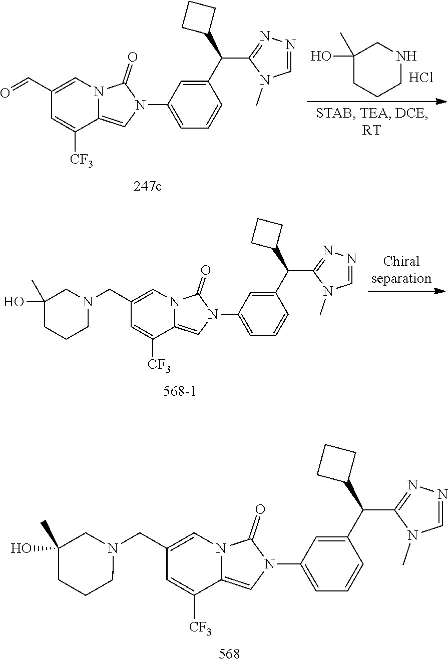

### 1. Synthesis of 568-1

To a stirred solution/mixture of 247c (1 g, 2.197 mmol, 1 equiv) and 3-methylpiperidin-3-ol hydrochloride (0.50 g, 3,296 mmol, 1.5 equiv) in DCE (15 mL) was added TEA (1000 g 9.882 mmol, 3 equiv) at room temperature. The resulting mixture was stirred for 1 h at room temperature. To the above mixture was added STAB (1.40 g, 6.588 mmol 2 equiv) at room temperature. The resulting mixture was stirred overnight at room temperature. The reaction was quenched with saturated NaHCO₃ (aq.) (80 mL) at room temperature. The aqueous layer was extracted with CH₂Cl₂ (3×40 mL). The resulting mixture was concentrated under reduced pressure. The residue was purified by Prep-TLC (CH₂Cl₂/MeOH 20:1) to afford 568-1 (300 mg, 24.65%) as a yellow solid.

### Example 561. Synthesis of Compound 569

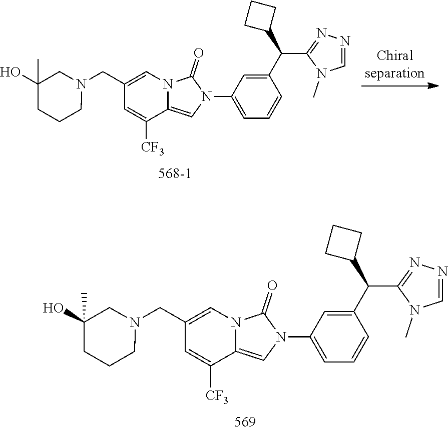 [SMILES: Cn1cnnc1[C@@H](c1cccc(-n2cc3c(C(F)(F)F)cc(CN4CCCC(C)(O)C4)cn3c2=O)c1)C1CCC1 ; Cn1cnnc1[C@@H](c1cccc(-n2cc3c(C(F)(F)F)cc(CN4CCC[C@@](C)(O)C4)cn3c2=O)c1)C1CCC1]

### 1. Synthesis of 569

The 568-1 (300 mg) was purified by chiral separation with the following conditions (Column: CHIRALPAK IG, 2*25 cm, 5 um; Mobile Phase A: Hex (0.5% 2M NH₃-MeOH), Mobile Phase B: EtOH:DCM=1:1; Flow rate: 20 mL/min; Gradient: 50% B to 50% B in 12 min; Wave Length: 220/254 nm; RT1 (min): 7.83; RT2 (min): 9.70; the second peak is product) to afford 569 (114.7 mg, 58.57%) as a yellow solid.

LC-MS-569: (ES, m/z): [M+H]⁺ 555

H-NMR-569: (400 MHz, DMSO-d₆, δ ppm): 1.12 (s, 3H), 1.31-1.58 (m, 3H), 1.61-1.73 (m, 2H), 1.73-1.87 (m, 4H), 2.08-2.10 (m, 2H), 2.21-2.23 (m, 2H), 2.33-2.39 (m, 1H), 3.19-3.25 (m, 2H), 3.25-3.33 (m, 1H), 3.46 (s, 3H), 4.24-4.27 (m, 2H), 7.07 (s, 1H), 7.18-7.20 (d, 1H), 7.31 (s, 1H), 7.42-7.46 (m, 1H), 7.68-7.71 (d, 1H), 7.74 (s, 1H), 8.33 (s, 1H).

### Example 562. Synthesis of Compound 570

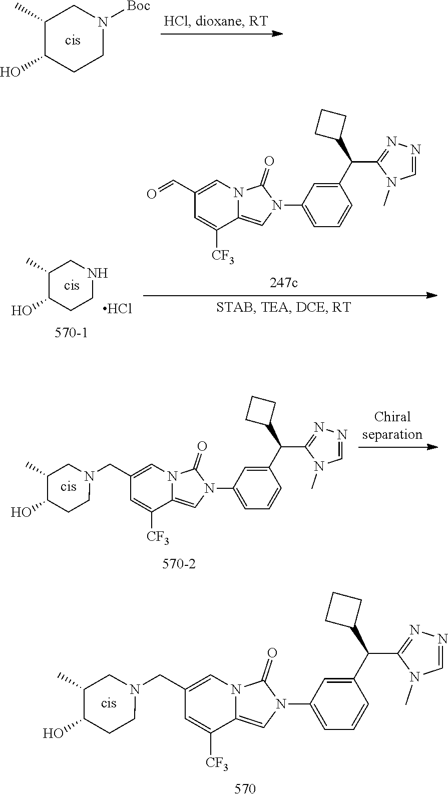

### 1. Synthesis of 570-1

Into a 100 mL round-bottom flask were added tert-butyl 4-hydroxy-3-methylpiperidine-1-carboxylate (1 g, 4.645 mmol, 1 equiv) and HCl (gas) in 1,4-dioxane (20 mL) at room temperature. The resulting mixture was stirred overnight at room temperature. The resulting mixture was concentrated under vacuum. This resulted in 570-1 (1 g, 99.39%) as a light yellow solid. The crude product was used in the next step directly without further purification.

### 2. Synthesis of 570-2

To a stirred solution of 570-1 (1000 mg, 6.595 mmol, 1 equiv) and 247c (1501.82 mg, 3.297 mmol, 0.5 equiv) in DCE (10 mL) was added TEA (1334.74 mg, 13.190 mmol, 2 equiv) at room temperature. The resulting mixture was stirred for 4 h at room temperature. To the above mixture was added STAB (2795.49 mg, 13.190 mmol, 2 equiv) at room temperature. The resulting mixture was stirred for additional overnight at room temperature. The reaction was quenched with NaHCO₃ (aq.) at room temperature. The aqueous layer was extracted with DCM (5×30 mL). The resulting mixture was concentrated under vacuum. The residue was purified by Prep-TLC (DCM/MeOH 15:1) to afford 570-2 (270 mg, 7.09%) as a yellow solid.

### 3. Synthesis of 570

The 570-1 (270 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 70% B to 70% B in 21 min; Wave Length: 220/254 nm; RT1 (min): 13.65; RT2 (min): 17.62; the first peak is product) to afford 570 (88.5 mg, 32.29%) as a yellow solid.

LC-MS-570: (ES, m/z): [M+H]⁺ 555

H-NMR-570: (400 MHz, DMSO-d6, δ ppm): 0.79-0.86 (d, 3H), 1.59-1.68 (m, 2H), 1.70-1.91 (m, 6H), 2.00-2.45 (m, 5H), 3.19-3.33 (m, 3H), 3.20-3.43 (s, 3H), 3.59-3.60 (m, 1H), 4.25-4.27 (d, 1H), 4.38-4.40 (m, 1H), 7.01 (s, 1H), 7.18-7.20 (d, 1H), 7.31 (s, 1H), 7.42-7.46 (m, 1H), 7.66-7.70 (m, 2H), 7.74 (s, 1H), 8.34 (s, 1H).

### Example 563. Synthesis of Compound 571

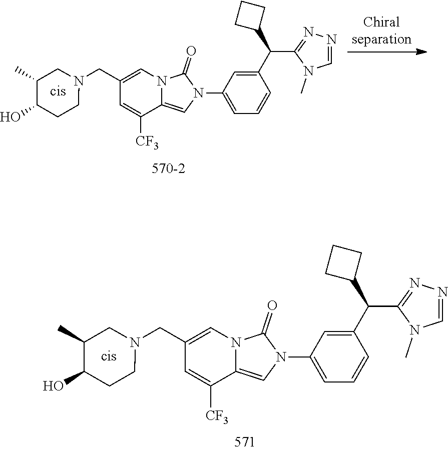 [SMILES: C[C@@H]1CN(Cc2cc(C(F)(F)F)c3cn(-c4cccc([C@H](c5nncn5C)C5CCC5)c4)c(=O)n3c2)CC[C@@H]1O ; C[C@H]1CN(Cc2cc(C(F)(F)F)c3cn(-c4cccc([C@H](c5nncn5C)C5CCC5)c4)c(=O)n3c2)CC[C@H]1O]

### 1. Synthesis of 571

The 570-2 (270 mg) was purified by Prep-Chiral-HPLC with the following conditions (Column: CHIRALPAK IC, 2*25 cm 5 μm; Mobile Phase A: Hex (0.5% 2M NH₃-MeOH), Mobile Phase B: EtOH:DCM=1:1; Flow rate: 20 mL/min; Gradient: 70% B to 70% B in 21 min; Wave Length: 220/254 nm; RT1 (min): 13.65; RT2 (min): 17.62; the second peak is product) to afford 571 (82.1 mg, 29.83%) as a yellow solid.

LC-MS-571: (ES, m/z): [M+H]⁺ 555

H-NMR-571: (400 MHz, DMSO-d6, δ ppm): 0.79-0.86 (d, 3H), 1.59-1.91 (m, 8H), 1.95-2.15 (m, 2H), 2.15-2.49 (m, 3H), 3.19-3.33 (m, 3H), 3.20-3.43 (s, 3H), 3.59-3.60 (m, 1H), 4.25-4.27 (d, 1H), 4.38-4.40 (m, 1H), 7.02 (s, 1H), 7.18-7.20 (d, 1H), 7.31 (s, 1H), 7.42-7.46 (m, 1H), 7.68-7.70 (m, 2H), 7.73 (s, 1H), 8.33 (s, 1H).

### Example 564. Synthesis of Compound 572

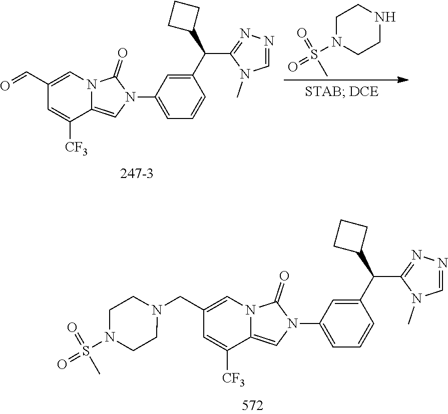

### 1. Synthesis of 572

Into a 40 mL sealed tube were added 1-methanesulfonylpiperazine (400 mg, 2.436 mmol, 2 equiv) DCE (4 mL) and 247-3 (554.67 ng, 1.218 mmol, 1 equiv) at room temperature. The resulting mixture was stirred for 1 h at room temperature. To the above mixture, was added STAB (516.23 mg, 2.436 mmol, 2 equiv) at room temperature. The resulting mixture was stirred for additional 2 h at room temperature. The reaction was quenched by the addition of sat. NH₄Cl (aq.) (10 mL) at room temperature. The aqueous layer was extracted with CH₂Cl₂:MEOH (10:1) (3×10 mL), The resulting mixture was concentrated under vacuum. The residue was purified by reverse flash chromatography with the following conditions: column, C18 silica gel; mobile phase, MeCN in Water (0.1% FA), 5% to 65% gradient in 30 min; detector, UV 254 nm. This resulted in 572 (136.5 mg, 18.20%) as a yellow solid.

LC-MS-572: (ES, m/z): [M+H]⁺ 604

H-NMR-572: (400 MHz, CD₃OD, δ ppm): 1.73-1.82 (m, 1H), 1.90-1.98 (m, 4H), 2.24-2.29 (m, H), 2.61-2.66 (m, 4H), 2.87 (s, 3H), 3.15-3.28 (m, 5H), 3.42 (s, 2H), 3.53 (s, 3H), 4.29-4.31 (m, 1H), 7.10-7.13 (d, 2H), 7.27-7.29 (d, 1H), 7.48-7.52 (t, 1H), 7.64-7.71 (m, 3H), 8.28 (s, 1H).

### Example 565. Synthesis of Compound 573_P1

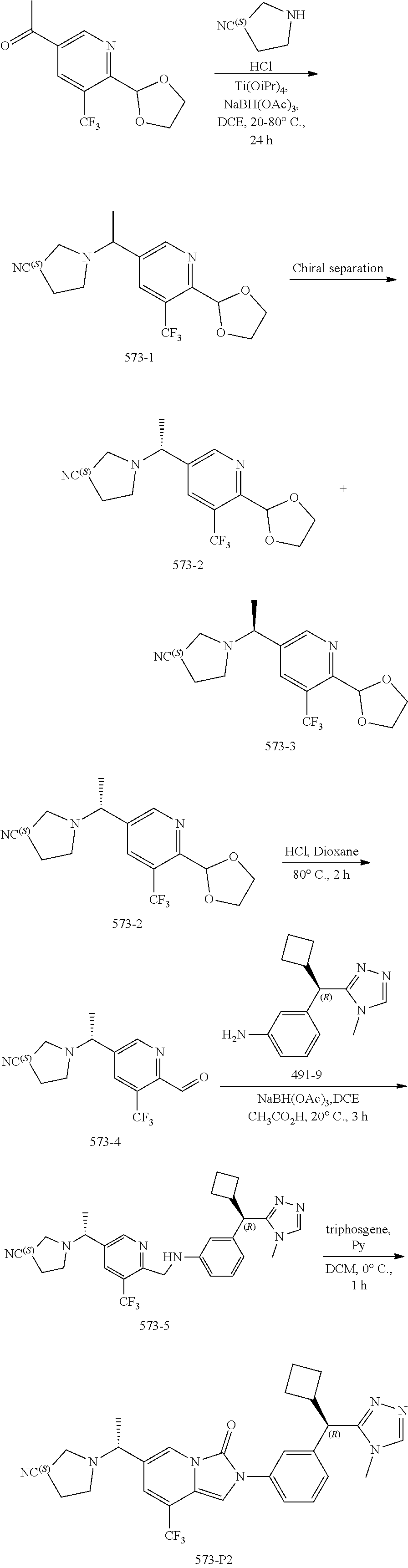

### 1. Synthesis of 573-1

To a solution of 1-(6-(1,3-dioxolan-2-yl)-5-(trifluoromethyl)pyridin-3-yl)ethanone (1.1 g, 4.21 mmol, 1 equiv) and (38)-pyrrolidine-3-carbonitrile (837 mg, 6.32 m mol, 1.5 equiv, HCl) in DCE (20 mL) was added tetraisopropoxytitanium (3.59 g, 12.63 mmol, 3.73 mL, 3 equiv)e The mixture was heated to 80° C. and stirred at 80° C. for 12 hr. Then sodium;triacetoxyboranuide (3.57 g, 16.85 mm ol, 4 equiv) was added to reaction mixture at 20° C. The mixture was stirred at 80° C. for 12 hr under N2. The mixture was poured into NaHCO₃ aq. (20 mL), extracted with EtOAc (10 mL×3). The combined organic layer was washed with brine (30 mL), dried over anhydrous Na2SO4, filtered and concentrated under reduced pressure to give a crude. The residue was purified by column chromatography (SiO2, Petroleum ether/Ethyl acetate=50/1 to 0/1) to afford 573-1 (480 mg, 33.33%) as a yellow oil.

### 2. Synthesis of 573-2 and 573-3

573-1 (480 mg) was purified by Chiral separation with the following conditions (column: DAICEL CHIRALCEL OD (250 mm*30 mm, 10 um); mobile phase: [Neu-IPA]; B %: 26%-26%, 7 min Flow rate: 2.5 m/min; Wave Length: 220/254 nm; RT1 (min): 2.142; RT2 (min): 2.487) to afford 573-2 (155 ng, 32.29%) as a yellow oil and 573-3 (220 ng, 45.83%) as a yellow oil,

1H-NMR-573-2: (400 MHz, METHANOL-d4) δ 8.80 (d, J=1.6 Hz, 1H), 8.15 (d, J=2.0 Hz, 1H), 6.17 (d, J=1.2 Hz, 1H), 4.29-4.21 (m, 2H), 4.11-4.05 (m, 2H), 3.62 (q, J=6.8 Hz, 1H), 3.21-3.18 (m, 1H), 2.91-2.87 (m, 1H), 2.75-2.71 (m, 1H), 2.67-2.57 (m, 2H), 2.30-2.24 (m, 1H), 2.09-2.02 (m, 1H), 1.44 (d, J=6.8 Hz, 3H).

1H-NMR-573-3: (400 MHz, METHANOL-d4) δ=8.80 (d, J=1.2 Hz, 1H), 8.15 (d, J=1.6 Hz, 1H), 6.17 (d, J=0.8 Hz, 1H), 4.27-4.21 (m, 2H), 4.11-4.05 (m, 2H), 3.62 (q, J=6.4 Hz, 1H), 3.21-3.16 (m, 1H), 2.91-2.82 (m, 2H), 2.66-2.62 (m, 1H), 2.49-2.43 (m, 1H), 2.29-2.24 (m, 1H), 2.11-2.03 (m, 1H), 1.44 (d, J=6.4 Hz, 3H).

### 3. Synthesis of 573-4

To a solution of 573-2 (155 mg, 454 umol, 1 equiv) in dioxane (1 mL) was added HCl (4M, 1 mL). The mixture was heated to 80° C. and stirred at 80° C. for 2 hr. The mixture was poured into NaHCO₃ aq. (10 mL) adjust pH to 8-9, extracted with EtOAc (5 mL×3). The combined organic layer was washed with brine (10 mL), dried over anhydrous Na2SO4, concentrated in vacuum to give a residue, 573-4 (135 mg, crude) was obtained as a light-yellow oil.

### 4. Synthesis of 573-5

To a solution of 573-4 (135 mg, 454 umol, 1 equiv) in DCE (3 mL) was added 491-9 (110 mg, 454 umol, 1 equiv), acetic acid (27 mg, 454 umol, 25.97 uL, 1 equiv) at 20° C. The reaction was stirred for 1 h. Then NaBH(OAc)3 (193 mg, 908 umol, 2 equiv) was added and stirred at 20° C. for 2 hr. The mixture was poured into water (10 ml), extracted with DCM (5 ml*3). The combined organic layer was washed with brine (10 mL), dried over anhydrous Na2SO4, concentrated in vacuum to give a residue. The residue was purified by column chromatography (SiO2, Petroleum ether/Ethyl acetate=20/1 to 0/1) and (DCM/MeOH=50/l to 1/1) to afford 573-5 (200 mg, 84.11% yield) as a light-yellow solid.

1H-NMR-573-5: (400 MHz, METHANOL-d4) δ 8.70 (d, J=1.6 Hz, 1H), 8.31-8.29 (m, 1H), 8.10 (d, J=1.6 Hz, 1H), 7.07-7.03 (m, 1H), 6.59-6.56 (m, 1H), 6.51-6.49 (m, 1H), 6.47-6.45 (m, 1H), 4.52 (s, 2H), 3.98 (d, J=10.8 Hz, 1H), 3.58-3.53 (m, 1H), 3.42 (s, 3H), 3.22-3.15 (m, 2H), 2.88-2.84 (m, 1H), 2.74-2.70 (m, 1H), 2.67-2.55 (m, 2H), 2.29-2.19 (m, 2H), 2.08-2.01 (m, 1H), 1.89-1.70 (m, 5H), 1.43 (d, J=6.8 Hz, 3H).

### 5. Synthesis of 573_P1

To a solution of 573-5 (150 mg, 286.48 umol, 1 equiv) in DCM (3 mL) was added Py (135 mg, 1.72 mmol, 139 uL, 6 equiv) and bis(trichloromethyl) carbonate (0.11 g, 370 umol, 1.29 equiv). The mixture was stirred at 0° C. for 1 hr. It was combined with a small test scale (50 mg) together. The reaction mixture was diluted with NaHCO₃ aq. (15 ml), extracted with DCM (10 mL×3). The combined organic layer was washed with brine (10 mL), dried over anhydrous Na2SO4, concentrated in vacuum to give a residue. The residue was purified by prep-HPLC (column: Phenomenex Luna C18 200*40 mm*10 um; mobile phase: [water (PA)-ACN]; B %: 10%-45%, 8 min, Wave Length: 220 nm; RT1 (min): 7.7 min) to afford 573 P1 (63.8 mg, 30.38% yield) as a yellow solid.

MS-573_P1: (ES, m/z): [M+H]+ 550.2.

1H-NMR-573_P1: (400 MHz, METHANOL-d4) δ 8.37 (d, J=3.6 Hz, 1H), 7.69 (d, J=2.0 Hz, 1H), 7.65-7.62 (m, 2H), 7.50-7.46 (m, 1H), 7.26-7.24 (m, 1H), 7.13 (d, J=2.4 Hz, 2H), 4.29-4.26 (m, 1H), 3.54-3.50 (m, 3H), 3.26-3.19 (m, 2H), 2.87-2.83 (m, 2H), 2.70-2.67 (m, 2H), 2.27-2.24 (m, 2H), 2.08-2.04 (m, 1H), 1.95-1.80 (m, 5H), 1.78-1.73 (m, 1H), 1.41-1.35 (m, 3H).

### Example 566. Synthesis of Compound 573_P2

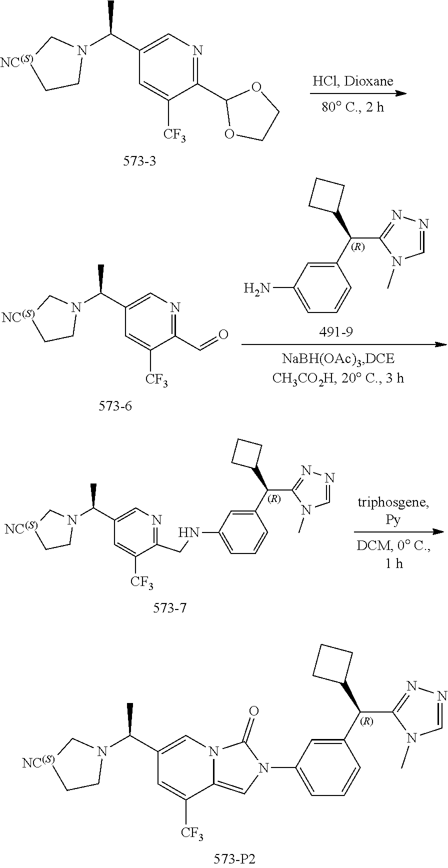

### 1. Synthesis of 573-6

To a solution of 573-3 (220 mg, 645 umol, 1 equiv) in dioxane (15 mL) was added HC (4M, 1.5 mL) The mixture was heated to 80° C. and stirred at 80° C. for 2 hr. The mixture was poured into NaHCO₃ aq. (10 mL) adjust pH to 8-9, extracted with EtOAc (5 mL×3). The combined organic layer was washed with brine (10 mL), dried over anhydrous Na2SO4, concentrated in vacuum to give a residue. 573-6 (185 mg, crude) was obtained as a light-yellow oil,

### 2. Synthesis of 573-7

To a solution of 573-6 (185 mg, 622 umol, 1 equiv) in DCE (3 ml) was added 491-9 (151 mg, 622 umol, 1 equiv), CH3COOH (37.37 mg, 622.32 umol, 35.63 uL, 1 equiv) at 20° C. stirred for 1 hr. Then NaBH(OAc)3 (264 mg, 1.24 mmol, 2 equiv) was added and stirred at 20° C. for 2 hr. The mixture was poured into water (10 ml), extracted with DCM (5 ml*3). The combined organic layer was washed with brine (10 mL), dried over anhydrous Na2SO4, concentrated in vacuum to give a residue. The residue was purified by column chromatography (SiO2, Petroleum ether/Ethyl acetate=20/1 to 0/1) and (DCM/MeOH=50/1 to 1/1) to afford 573-7 (290 mg, 89.00% yield) as a light-yellow solid.

1H-NMR-573-7: (400 MHz, METHANOL-d4) δ 8.70 (s, 1H), 8.31-8.30 (m, 1H), 8.10 (d, J=1.2 Hz, 1H), 7.07-7.03 (m, 1H), 6.59-6.57 (m, 1H), 6.51-6.49 (m, 1H), 6.47-6.45 (m, 1H), 4.52 (s, 2H), 3.98 (d, J=10.8 Hz, 1H), 3.58-3.52 (m, 1H), 3.41 (s, 3H), 3.21-3.14 (m, 2H), 2.89-2.82 (m, 2H), 2.64-2.61 (m, 1H), 2.47-2.41 (m, 1H), 2.31-2.19 (m, 2H), 2.10-2.04 (m, 1H), 1.89-1.70 (m, 5H), 1.42 (d, J=6.8 Hz, 3H).

### 3. Synthesis of 573_P2

To a solution of 573-7 (240 mg, 458 umol, 1 equiv) in DCM (4 mL) was added Py (218 mg, 2.75 mmol, 222 uL, 6 equiv) and bis(trichloromethyl) carbonate (0.07 g, 235.89 umol, 0.5 equiv). The mixture was stirred at 0° C. for 1 hr. It was combined with a small test scale (50 mg) together. The reaction mixture was diluted with NaHCO₃ aq. (15 mL), extracted with DCM (10 mL×3). The combined organic layer was washed with brine (10 mL), dried over anhydrous Na2SO4, concentrated in vacuum to give a residue. The residue was purified by prep-HPLC (column: Phenomenex Luna C18 200*40 mm*10 um; mobile phase: [water (FA)-ACN]; B %: 10%-45%, 8 min, Wave Length: 220 nm; RT1 (min): 7.7 min) to afford 573_P2 (99.4 mg, 32.65% yield) as a yellow solid.

MS-573_P2: (ES, m/z): [M+H]+ 550.2.

1H-NMR-573_P2: (400 MHz, METHANOL-d4) δ 8.36 (s, 1H), 7.68 (s, 1H), 7.65-7.64 (m, 1H), 7.62 (s, 1H), 7.49 (t, J=8.0 Hz, 1H), 7.25 (d, J=7.6 Hz, 1H), 7.13 (s, 1H), 7.12 (s, 1H), 4.28 (d, J=10.4, 1H), 3.53 (s, 3H), 3.27-3.17 (m, 2H), 2.95-2.91 (m, 1H), 2.86-2.82 (m, 1H), 2.74-2.70 (m, 1H), 2.57-2.51 (m, 1H), 2.30-2.23 (m, 2H), 2.10-2.05 (m, 1H), 1.95-1.85 (m, 5H), 1.80-1.73 (m, 1H), 1.39 (d, J=6.8 Hz, 3H).

### Example 567. Synthesis of Compound 574

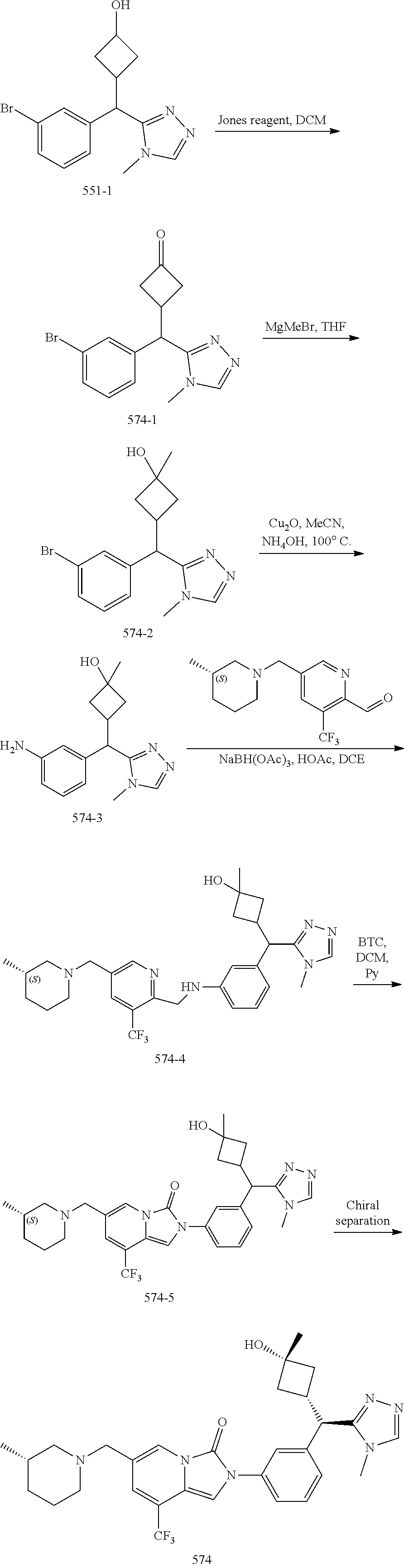

### 1. Synthesis of 574-1

To a stirred solution of 551-1 (2.4 g, 7.449 mmol, 1 equiv) in DCM (30 mL) was added Jones reagent (7.45 mL, 14.898 mmol, 2 equiv, 2 M) dropwise at 0° C. The resulting mixture was stirred for 2 h at 0° C. The reaction was quenched by the addition of sat. NaHCO₃ (aq.) (30 mL). The resulting mixture was filtered, the filter cake was washed with DCM (5 mL). The resulting mixture was extracted with CH Cl₂ (3×30 mL). The combined organic layers were dried over anhydrous Na₂SO₄. After filtration, the filtrate was concentrated under reduced pressure. This resulted in 574-1 (1.8 g, 75.47%) as a white solid.

### 2. Synthesis of 574-2

To a stirred solution of 574-1 (1.8 g, 5.622 mmol, 1 equiv) in THF (20 mL) was added MeMgBr (6.75 mL, 6.746 mmol, 1.2 equiv, 1M in THF) dropwise at room temperature under nitrogen atmosphere. The resulting mixture was stirred for 3 h at room temperature under nitrogen atmosphere. The reaction was quenched by the addition of sat. NH₄Cl (aq.) (60 mL) at room temperature. The resulting mixture was extracted with EtOAc (3×50 mL). The combined organic layers were concentrated under reduced pressure. The residue was purified by reverse flash chromatography with the following conditions: column, C18 silica gel; mobile phase, MeCN in Water (10 mmol/L NH4HCO3), 5% to 70% gradient in 30 min; detector, UV 220 nm. This resulted in 574-2 (1.6 g, 84.65%) as a white solid,

### 3. Synthesis of 574-3

To a solution of 574-2 (1.6 g, 4.759 mmol, 1 equiv) in MeCN (48 mL) and NH40H (48 mL) was Cu₂O (0.27 g, 1,904 mmol, 0.4 equiv) in a pressure tank. The resulting mixture was stirred for overnight at 100° C. The resulting mixture was diluted with DCM (100 mL). The resulting mixture was filtered, the filter cake was washed with DCM (5 mL). The resulting mixture was extracted with CH2Cl₂ (2×30 mL). The combined organic layers were concentrated under reduced pressure. The residue was purified by silica gel column chromatography, eluted with CH₂Cl₂/MeOH (20:1) to afford 574-3 (1.2 g, 92.59%) as a light yellow solid.

### 4. Synthesis of 574-4

To a stirred solution of 574-3 (1.1 g, 4.039 mmol, 1 equiv) and 1-2 (1.39 g, 4.847 mmol, 1.2 equiv) in DCE (20 mL) was added NaBH(OAc)₃ (1.71 g, 8.078 mmol, 2 equiv). The resulting mixture was stirred for overnight at room temperature under nitrogen atmosphere. The reaction was quenched by the addition of sat. NaHCO₃ (aq.) (30 mL) at room temperature. The resulting mixture was extracted with CH₂Cl₂ (3×30 mL). The combined organic layers were concentrated under reduced pressure. The residue was purified by silica gel column chromatography, eluted with CH₂Cl₂/MeOH (15:1) to afford 574-4 (1.2 g, 54.75%) as a white solid.

### 5. Synthesis of 574-5

To a stirred solution of 574-4 (1.2 g, 2.211 mmol, 1 equiv) and Pyridine (1.05 g, 13.266 mmol, 6 equiv) in DCM (24 mL) was added Triphosgene (0.26 g, 0.884 mmol, 0.4 equiv) at 0° C. under nitrogen atmosphere. The resulting mixture was stirred for 3 h at 0° C. under nitrogen atmosphere. The reaction was quenched by the addition of sat. NaHCO₃ (aq.) (30 mL). The resulting mixture was extracted with CH2Cl2/MeOH (3×30 mL). The combined organic layers were concentrated under reduced pressure. The residue was purified by reverse flash chromatography with the following conditions: column, C18 silica gel; mobile phase, MeCN in Water (10 mmol/L NH4HCO3) 10% to 75% gradient in 30 min; detector, UV 254 m. This resulted in 574-5 (605 mg, 48.11%) as a yellow solid.

### 6. Synthesis of 574

574-5 (815 mg, 1,433 mmol, 1 equiv) was purified by Prep-Chiral-SFC with the following conditions (Column: CHIRAL ART Cellulose-SB, 3*25 cm, 5 μm; Mobile Phase A: CO₂, Mobile Phase B: MeOH:DCM=1:1 (0.1% 2M NH₃-MEOH); Flow rate: 100 mL/min; Gradient: isocratic 35% B; Column Temperature (° C.): 35; Back Pressure (bar): 100; Wave Length: 220 nm; RT1 (min): 2.55; RT2 (m in): 2.68; RT3 (min): 3.02; RT4 (min): 4.37; the second peak was product) to afford crude product 152 mg. The crude product (152 rug) was purified by Prep-Chiral-HPLC with the following conditions (Column: CHIRAL ART Amylose-SA, 2*25 cm, 5 μm; Mobile Phase A: Hex (0.5% 2M NH3-MeOH), Mobile Phase B: EtOH:DCM=1:1; Flow rate: 20 mL/min; Gradient: 20% B to 20% B in 11 min; Wave Length: 220/254 nm; RT1 (min): 7.35; RT2 (min): 8.72; the second peak was product) to afford 574 (156.2 mg, 19.17%) as a yellow solid.

LCMS: (ES, m/z): [M+H]⁺ 569. NMR 0: (400 MHz, CD₃OD, δ ppm): 0.90-0.97 (m, 4H), 1.42 (s, 3H), 1.61-1.84 (m, 6H), 1.93-2.01 (m, 3H), 2.32-2.37 (m, 1H), 2.79-2.90 (m, 3H), 3.30 (s, 2H), 3.55 (s, 3H), 4.22-4.25 (d, 1H), 7.12 (s, 2H), 7.26-7.28 (d, 1H), 7.49-7.53 (t, 1H), 7.66-7.72 (m, 3H), 8.38 (s, 1H).

### Example 56. Synthesis of Compound 575

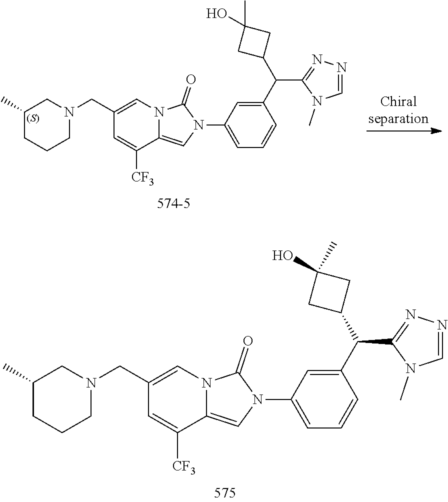

### 1. Synthesis of 575

574-5 (815 mg) was purified by Prep-Chiral-SFC with the following conditions (Column: CHIRAL ART Cellulose-SB, 3*25 cm, 5 μm; Mobile Phase A: CO2, Mobile Phase B: MeOH:DCM=1:1 (0.1% 2M NH3-MEOH); Flow rate: 100 mL/min; Gradient: isocratic 35% B; Column Temperature (° C.): 35; Back Pressure (bar): 100; Wave Length: 220 nm; RT1 (min): 2.55; RT2 (main): 2.68; RT3 (min): 3.02; RT4 (min): 4.37; the first peak was product) to afford crude product 98 mg. The crude product (98 mg) was purified by Prep-HPLC with the following conditions (Column: CHIRALPAK TG, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 8 min; Wave Length: 220/254 nm; RT1 (min): 4.93; RT2 (min): 6.56; the first peak was product) to afford 575 (14.0 mg, 1.72%) as a yellow solid.

LCMS-575: (ES, m/z): [M+H]+ 569 NMR-575: (400 MHz, CD3OD, δ ppm): 0.90-0.97 (m, 4H), 1.42 (s, 3H), 1.61-1.85 (m, 6H), 1.94-2.06 (m, 3H), 2.34-2.40 (m, 1H), 2.84-2.90 (m, 2H), 3.32 (s, 2H), 3.38-3.42 (m, 1H), 3.55 (s, 3H), 4.24-4.27 (d, 1H), 7.12 (s, 2H), 7.27-7.29 (d, 1H), 7.48-7.52 (t, 1H), 7.65-7.70 (m, 3H), 8.37 (s, 1H).

### Example 569. Synthesis of Compound 576

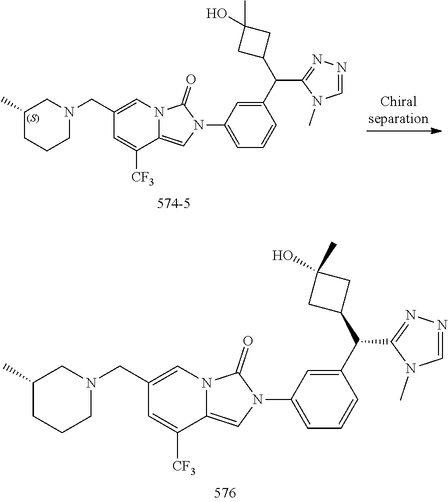

### 1. Synthesis of 576

574-5 (815 ng) was purified by Prep-HPLC with the following conditions (Column: CHIRAL ART Celluose-SB, 3*25 cm, 5 μm; Mobile Phase A: CO₂, Mobile Phase B: MeOH:DCM=1:1 (0.1% 2M NH₃-MEOH); Flow rate: 100 mL/min; Gradient: isocratic 35% B; Column Temperature (° C.): 35; Back Pressure (bar): 100; Wave Length: 220 nm; RT1 (min): 2.55; RT2 (min): 2.68; RT3 (mini): 3.02; RT4 (min): 4.37; the third peak was product) to afford crude product 20 mg. The crude product (20 mg) was purified by Prep-Chiral-HPLC with the following conditions (Column: CHIRALPAK IC, 2*25 cm, 5 μm; Mobile Phase A: Hex (05% 2M NH₃-MeOH), Mobile Phase B: EtOH:DCM=1:1; Flow rate: 20 mL/min; Gradient: 40% B to 40% B in 15 min; Wave Length: 220/254 nm; RT1 (min): 9.90; RT2 (min): 12.6; Sample Solvent: EtOH:DCM=1:1; the second peak was product) to afford 2 576 (16.6 mg, 2.04%) as a yellow solid.

LCMS-576: (ES, m/z): [M+H]⁺ 569

NMR-576: (400 MHz, CD₃OD, δ ppm): 0.89-0.95 (m, 4H), 1.35 (s, 3H), 1.59-1.85 (m, 6H), 1.94-2.04 (m, 3H), 2.34-2.39 (m, 1H), 2.85-2.91 (m, 3H), 3.33 (s, 2H), 3.40-3.43 (m, 1H), 3.55 (s, 3H), 4.24-4.27 (d, 1H), 7.12 (s, 2H), 7.27-7.29 (d, 1H), 7.48-7.52 (t, 1H), 7.66-7.69 (m, 3H), 8.37 (s, 1H).

### Example 570. Synthesis of Compound 577

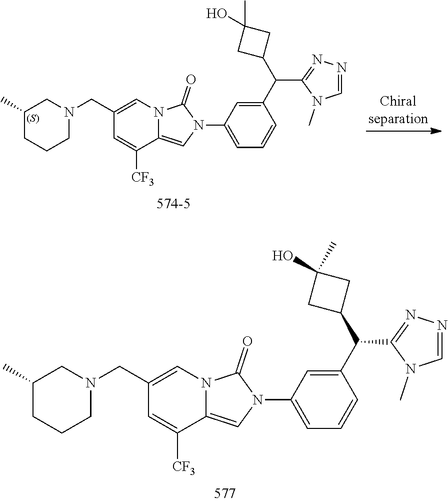

### 1. Synthesis of 577

574-5 (815 mg) was purified by Prep-Chiral-SFC with the following conditions (Column: CHIRAL ART Cellulose-SB, 3*25 cm, 5 μm; Mobile Phase A: CO2, Mobile Phase B: MeOH:DCM=1:1 (0.1% 2M NH3-MEOH); Flow rate: 100 mL/min; Gradient: isocratic 35% B; Column Temperature (° C.): 35; Back Pressure (bar): 100; Wave Length: 220 nm; RT1 (min): 255; RT2 (min): 2.68; RT3 (min): 3.02; RT4 (min): 437; the fourth peak was product) to afford 577 (217.5 mg, 26.69%) as a yellow solid.

LCMS-577: (ES, m/z): [M+H]⁺ 569

NMR-577: (400 MHz, CD₃OD, δ ppm): 0.90-0.97 (m, 4H), 1.37 (s, 3H), 1.60-1.83 (m, 6H), 1.96-2.05 (m, 3H), 2.33-2.37 (m, 1H), 2.79-2.90 (m, 3H), 3.32 (s, 2H), 3.55 (s, 3H), 4.22-4.25 (d, 1H), 7.12 (s, 2H), 7.26-7.28 (d, 1H), 7.49-7.53 (t, 1H), 7.65-7.70 (m, 3H), 8.38 (s, 1H).

### Example 571. Synthesis of Compound 578

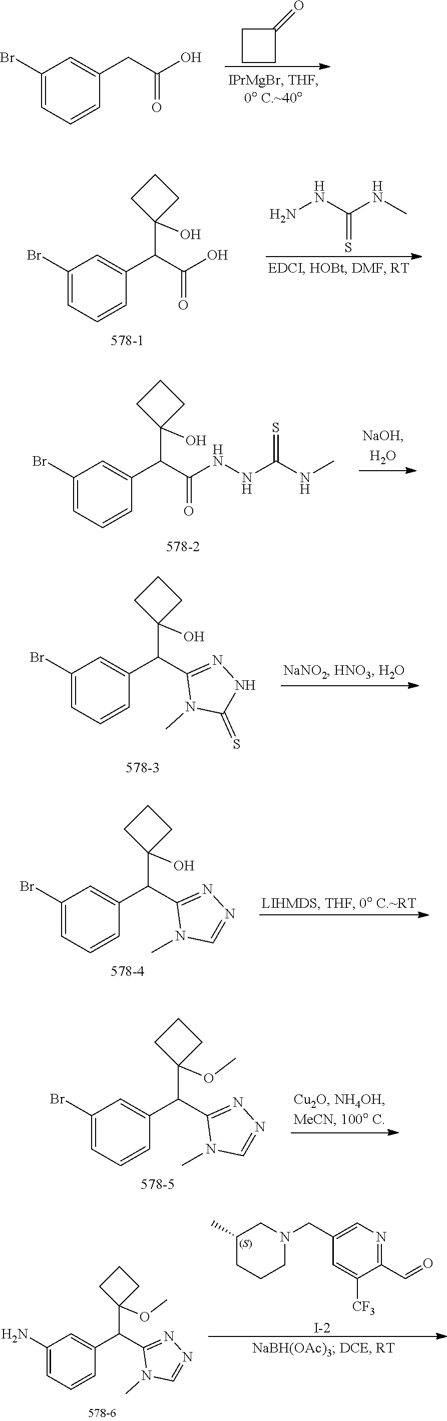

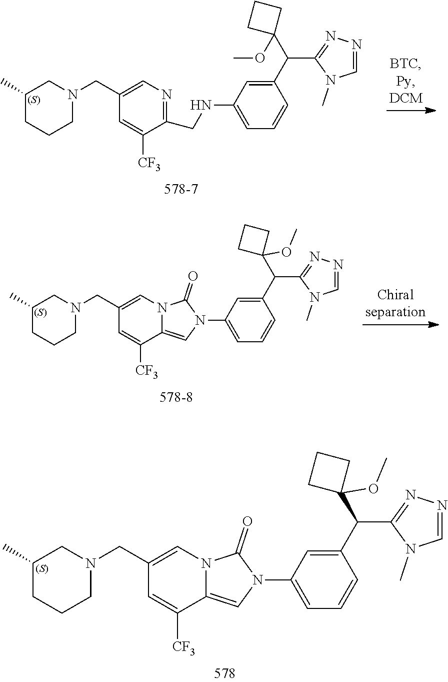

### 1. Synthesis of 578-1

Into a 1 L 3-necked round-bottom flask were added mi-bromophenylacetic acid (50 g, 232.508 mmol, 1 equiv) and THF (500 mL) at 0° C. To the above mixture was added cyclobutanone (17.93 g, 255,759 mmol, 1.1 equiv) at 40° C. The resulting mixture was stirred for additional 2 h at 40′° C. The reaction was quenched with sat, NH₄HCl (aq.)(1500 mL) at room temperature. The aqueous layer was extracted with EtOAc (3×1 L). The resulting mixture was concentrated under reduced pressure. This resulted in 579-1 (53 g, crude) as a yellow solid.

### 2. Synthesis of 578-2

Into a 1000 mL 3-necked round-bottom flask were added 579-1 (53 g, 185.876 mmol, 1 equiv), 1-amino-3-methylthiourea (23.46 g, 223.051 mmol, 1.2 equiv), HOBt (37.67 g, 278.814 mmol, 1.5 equiv) and DMF (530 ml) at room temperature. The resulting mixture was stirred for 1 h at room temperature under nitrogen atmosphere. To the above mixture was added EDCI (53.45 g, 278.814 mmol, 1.5 equiv) at room temperature. The resulting mixture was stirred for additional overnight at room temperature. The reaction was quenched by the addition of sat. NH4Cl (aq.) (1000 mL) at room temperature. The aqueous layer was extracted with EtOAc (3×500 mL). The resulting mixture was concentrated under vacuum. This resulted in 578-2 (65 g, crude) as a yellow solid.

### 3. Synthesis of 578-3

Into a 1.00 mL 3-necked round-bottom flask were added 578-2 (65 g, 174.600 mmol, 1 equiv), H2O (700 mL) and NaOH (27.93 g, 698.400 mmol, 4 equiv) at room temperature. The resulting mixture was stirred for overnight at room temperature. The mixture was acidified to pH 7 with HCl (1M). The aqueous layer was extracted with EtOAc (3×500 mL). The resulting mixture was concentrated under vacuum. The residue was purified by silica gel column chromatography, eluted with PE/EA (2:1) to afford 578-3 (33 g, 37.35%) as a white solid.

### 4. Synthesis of 578-4

Into a 1 L 3-necked round-bottom flask were added 5 578-3 (33 g, 93.149 mmol, 1 equiv), NaNO2, (64.27 g, 931.490 mmol, 10 equiv) and H₂O (200 mL) at room temperature. To the above mixture was added HNO₃ (465.7 ml, 465.745 mmol, 5 equiv, 1M) dropwise over 30 min at 0° C. The resulting mixture was stirred for additional 5 h at room temperature. The reaction was quenched with sat. NaHCO3 (aq.) at room temperature. The aqueous layer was extracted with EtOAc (3×1 L). The resulting mixture was concentrated under reduced pressure. The residue was purified by silica gel column chromatography, eluted with DCM/MeOH (30:1) to afford 578-4 (12 g, 37.98%) as a white solid.

### 5. Synthesis of 578-5

Into a 250 mL 3-necked round-bottom flask were added 578-4 (12 g, 37,243 mmol, 1 equiv) and THF (120 mL). To the above solution was added LiHMDS (18.70 g, 111,729 mmol, 3 equiv) at −40° C. under nitrogen atmosphere. The resulting mixture was stirred for 1 h at −40° C. under nitrogen atmosphere. To the above mixture was added MeI (10.57 g, 74.486 mmol, 2 equiv). The resulting mixture was stirred for additional overnight at room temperature. The reaction was quenched by the addition of sat. NH₄Cl (aq.) (300 mL) at room temperature. The aqueous layer was extracted with CH₂Cl₂ (3×100 mL). The resulting mixture was concentrated under reduced pressure. The residue was purified by reverse flash chromatography with the following conditions: column, C18 silica gel; mobile phase, MeCN in Water (10 mmol/L NH4HCO₃), 10% to 80% gradient in 30 min; detector, UV 220 nm. This resulted in 578-5 (1172 mg, 9.08%) as a white solid.

### 7. Synthesis of 578-6

Into a 100 ml, pressure tank reactor were added 578-5 (1172 mg, 3.486 mmol, 1 equiv), Cu₂O (249.38 mg, 1,743 mmol, 0.5 equiv), NH₄OH (20 mL) and MeCN (20 mL) at room temperature. The resulting mixture was stirred for overnight at 100° C. The resulting mixture was concentrated under reduced pressure. The residue was purified by reverse flash chromatography with the following conditions: column, C18 silica gel; mobile phase, MeCN in Water (0.1% NH₄HCO₃), 10% to 60% gradient in 40 min; detector, UN 220 nm. This resulted in 578-6 (390 mg, 36.97%) as a white solid.

### 8. Synthesis of 578-7

To a stirred solution of 578-6 (370 mg, 1,359 mmol, 1 equiv) and 1-2 (466.74 mg, 1.631 mmol, 1.2 equiv) in DCE (10 mL) was added NaBH(OAc)3 (575.86 mg, 2.718 mmol, 2 equiv). The resulting mixture was stirred for overnight at room temperature. The reaction was quenched by the addition of sat. NaHCO₃ (aq.) (15 mL). The resulting mixture was extracted with CH₂Cl₂/MeOH=10/1 (2×20 mL). The combined organic layers were concentrated under reduced pressure. The residue was purified by Prep-TLC (CH₂Cl₂/MeOH 25:1) to afford 578-7 (510 mg, 69.18%) as a white solid.

### 9. Synthesis of 578-8

To a stirred solution of 578-7 (490 mg, 0.903 mmol, 1 equiv) and Pyridine (428.55 mg, 5.418 mmol, 6 equiv) in DCM (10 mL) was added Triphosgene (107.18 mg, 0.361 mmol, 0.4 equiv) 0° C. under nitrogen atmosphere. The resulting mixture was stirred for 2 h at 0° C. under nitrogen atmosphere. The reaction was quenched by the addition of sat. NaHCO₃ (aq.) (15 mL). The resulting mixture was extracted with CH₂C2/MeOH=10/1 (2×20 mL). The combined organic layers were concentrated under reduced pressure. The residue was purified by reverse flash chromatography with the following conditions: column, C18 silica gel; mobile phase, MeCN in Water (10 mmol/L NH₄HCO₃), 10% to 65% gradient in 25 min; detector, UV 254 nm. This resulted in 578-8 (360 mg, 70.11%) as a yellow solid.

### 10. Synthesis of 578

578-8 (360 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 20 min; Wave Length: 220/254 nm; RT1 (min): 12.04; RT2 (min): 16.05; the first peak was product) to afford 578 (126.5 mg, 35.14%) as a yellow solid.

LCMS-578: (ES, m/z): [M+H]⁺ 569

NMR-578: (400 MHz, DMSO, δ ppm): 0.89-0.99 (m, 4H), 1.12-1.20 (m, 1H), 1.46-1.51 (m, 111), 1.58-1.74 (m, 5H), 1.80-1.91 (m, 1H), 2.11-2.20 (m, 1H), 2.27-2.36 (m, 3H), 2.71-2.82 (m, 2H), 3.25 (s, 3H), 3.26 (s, 2H), 3.51 (s, 3H), 4.66 (s, 1H), 7.02 (s, 2H), 7.25 (s, 1H), 7.45-7.49 (t, 1H), 7.56-7.57 (d, 1H), 7.67-7.72 (m, 2H), 8.03 (s, 1H), 8.36 (s, 1H).

### Example 572. Synthesis of Compound 579

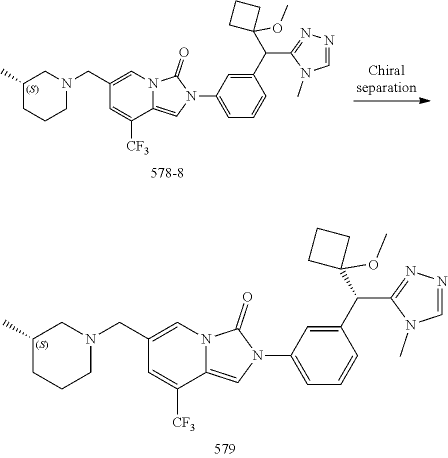

### 1. Synthesis of 579

578-8 (360 mg) was purified by Prep-Chiral-HPLC with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 20 min; Wave Length: 220/254 nm; RT1 (min): 12.04; RT2 (min): 16.05; the second peak was product) to afford 579 (126.5 mg, 35.14%) as a yellow solid.

LCMS-579: (ES, m/z): [M+H]⁺ 569

NMR-579: (400 MHz, DMSO, δ ppm): 0.82-0.99 (m, 4H), 1.10-1.17 (m, 1H), 1.44-1.50 (m, 1H), 1.59-1.67 (m, 5H), 1.90-1.92 (m, 1H), 2.17-2.27 (m, 2H), 2.29-2.38 (m, 2H), 2.76-2.82 (m, 2H), 3.25 (s, 3H), 3.26 (s, 2H), 3.51 (s, 3H), 4.66 (s, 1H), 7.02 (s, 2H), 7.25 (s, 1H), 7.45-7.49 (t, 1H), 7.56-7.58 (d, 1H), 7.68-7.73 (m, 2H), 8.03 (s, 1H), 8.36 (s, 1H).

### Example 573. Synthesis of Compound 580

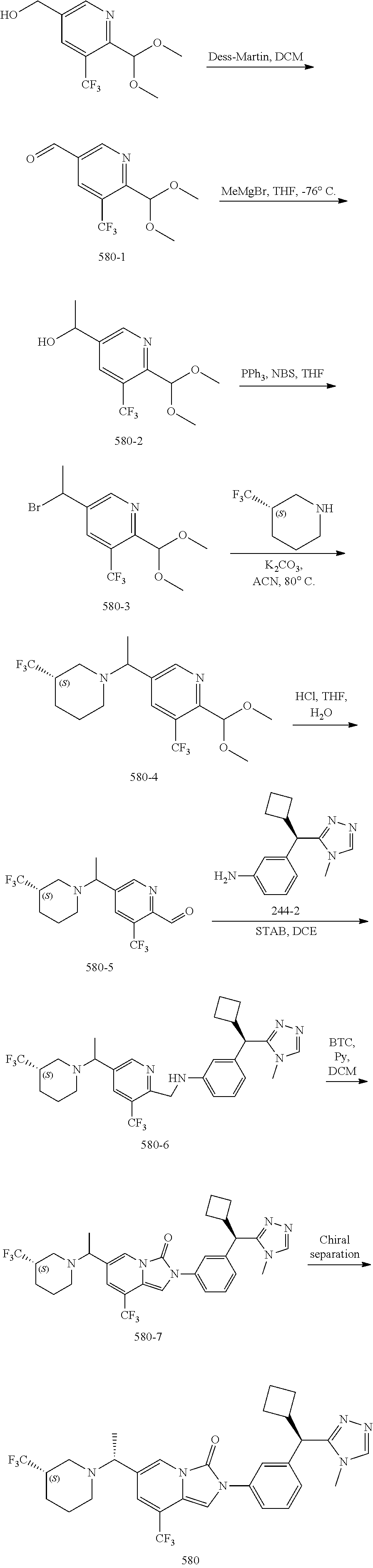

### 1. Synthesis of 580-1

Into a 100 mL 3-necked round-bottom flask were added (6-(dimethoxymethyl)-5-(trifluoromethyl)pyridin-3-yl)methanol (5 g, 19.904 mmol, 1 equiv), DCM (50 mL) and Dess-Martin (10.13 g, 23,885 mmol, 1.2 equiv) at 0° C. The resulting mixture was stirred for 4 h at room temperature. The reaction was quenched with sat. NaHCO₃ (aq.) (40 mL) at room temperature. The aqueous layer was extracted with CH₂Cl₂ (2×50 mL). The resulting mixture was concentrated under vacuum. The residue was purified by silica gel column chromatography, eluted with CH₂Cl₂/MeOH (100:1) to afford 580-1 (4 g, 80.65%) as a yellow oil.

### 2. Synthesis of 580-2

Into a 100 mL 3-necked round-bottom flask were added 580-1 (3.8 g, 15,249 mmol, 1 equiv) and THF (30 mL) at room temperature. To the above mixture was added MeMgBr (1M in THF) (15.2 mL) dropwise over 20 min at −78° C. The resulting mixture was stirred for additional overnight at room temperature. The reaction was quenched with sat. NH₄Cl (aq.) (40 mL) at 0° C. The aqueous layer was extracted with EtOAc (2×50 mL). The resulting mixture was concentrated under vacuum. The residue was purified by silica gel column chromatography, eluted with PE/EA (1:1) to afford 580-2 (2.7 g, 66.75%) as a yellow oil.

### 3. Synthesis of 580-3

Into a 100 mL 3-necked round-bottom flask were added 580-2 (2.8 g, 10,557 mmol, 1 equiv), THF (30 mL) and NBS (3.76 g, 21.114 mmol, 2 equiv) at room temperature. To the above mixture was added PPh₃ (5-54 g, 21.114 mmol, 2 equiv) at 0° C. The resulting mixture was stirred for additional overnight at room temperature. The reaction was quenched with water (30 mL) at room temperature. The aqueous layer was extracted with EtOAc (2×30 mL). The resulting mixture was concentrated under vacuum. The residue was purified by silica gel column chromatography, eluted with PE/EA (1:1) to afford 580-3 (2.2 g, 63.51%) as a yellow oil.

### 4. Synthesis of 580-4

Into a 50 mL 3-necked round-bottom flask were added 580-3 (510 mg, 1.554 mmol, 1 equiv), ACN (5 mL), (3S)-3-(trifluoromethyl)piperidine (261.84 mg, 1,709 mmol, 1.1 equiv) and K₂CO₃ (429.61 mg, 3.108 mmol, 2 equiv) at room temperature. The resulting mixture was stirred for overnight at 80° C. The reaction was quenched with sat. NH4Cl (aq.) (5 mL) at room temperature. The aqueous layer was extracted with EtOAc (2×5 mL). The resulting mixture was concentrated under vacuum. The residue was purified by Prep-TLC (PB/EA 1:1) to afford 580-4 (450 rug, 72.32%) as a yellow solid.

### 5. Synthesis of 580-5

Into a 50 mL 3-necked round-bottom flask were added 580-4 (500 mg, 1.249 mmol, 1 equiv), HCl (1M) (2.5 mL) and THF (2.5 mL) at room temperature. The resulting mixture was stirred for overnight at 60° C. The mixture was allowed to cool down to room temperature. The mixture was basified to pH 6 with saturated NaHCO₃ (aq.) (5 mL). The aqueous layer was extracted with EtOAc (3×5 mL). The resulting mixture was concentrated under vacuum. The residue was purified by Prep-TLC (CH₂Cl₂/MeOH 20:1) to afford 580-5 (400 mg, 90.40%) as a yellow oil.

### 6. Synthesis of 580-6

Into a 50 mL 3-necked round-bottom flask were added 580-5 (380 mg, 1.073 mmol, 1 equiv), DCE (4 mL) and 244-2 (259.91 mg, 1.073 mmol, 1 equiv) at room temperature. The resulting mixture was stirred for overnight at room temperature. To the above mixture was added STAB (454.63 mg, 2,146 mmol, 2 equiv) at room temperature. The resulting mixture was stirred for additional 3 h at room temperature. The reaction was quenched with sat. NaHCO₃ (aq.) (4 mL) at room temperature. The aqueous layer was extracted with CH Cl₂ (2×5 mL). The resulting mixture was concentrated under vacuum. The residue was purified by Prep-TLC (CH₂Cl2/MeOH 20:1) to afford 580-6 (410 mg, 65.84%) as a yellow solid.

### 7. Synthesis of 580-7

Into a 50 mL 3-necked round-bottom flask were added 580-6 (410 mg, 0.706 mmol, 1 equiv), DCM (8 ml) and pyridine (558.55 mg, 7.060 mmol, 10 equiv) at room temperature. To the above mixture was added triphosgene (73.34 mg, 0.247 mmol, 0.35 equiv) at 0° C. The resulting mixture was stirred for additional 30 min at room temperature. The reaction was quenched with sat. NaHCO₃ (aq.) (4 mL) at room temperature. The aqueous layer was extracted with CH₂Cl₂ (2×8 mL). The resulting mixture was concentrated under vacuum. The residue was purified by Prep-TLC (CH₂Cl₂/MeOH 20:1) to afford 580-7 (320 mg, 74.70%) as a yellow solid.

### 8. Synthesis of 580

The 580-7 (320 mg) was purified by Chiral separation with the following conditions (Column: (R, R)-WHELK-O1-Kromasi, 5*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 40% B in 18 min; Wave Length: 220/254 nm; RT1 (min): 9.83; RT2 (min): 13.99; The first peak in product; Sample Solvent: EtOH:DCM=1:1-HPLC; Injection Volume: 0.7 mL; Number of Runs: 6) to afford 580 (130.2 mg, 35.96%) as a yellow solid.

LC-MS-580: (ES, m/z): [M+H]⁺ 556

H-NMR-580: 1H NMR (400 MHz, DMSO-d6 ppm) δ 1.19-1.26 (m, 4H), δ 1.42-1.44 (d, 1H), δ 1.63-1.81 (m, 7H), δ 2.00-2.09 (m, 3H), δ 2.40-2.42 (d, 1H), δ 2.82-2.85 (d, 1H), δ 2.92-2.95 (d, 1H), δ 3.21-3.25 (m, 1H), δ 3.43 (s, 3H), 53.56-3.58 (d, 1H), δ 4.25-4.27 (d, 1H), δ 7.07 (s, 1H), δ 7.18-7.20 (d, 1H), δ 7.31 (s, 1H), δ 7.42-7.46 (t, 1H), δ 7.63-7.73 (m, 3H), δ 8.33 (s, 1H).

### Example 574. Synthesis of Compound 581

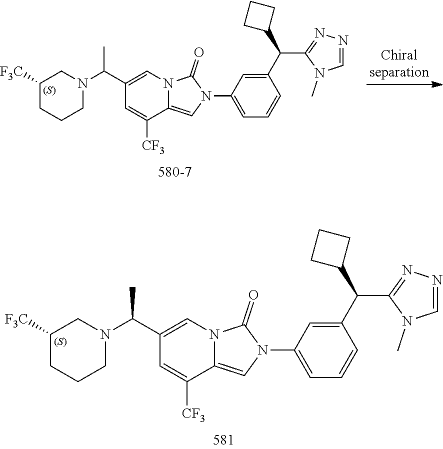

### 1. Synthesis of 581

The 580-7 (320 mg) was purified by Chiral separation with the following conditions (Column: (R, R)-WHELK-O1-Kromasi, 5*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 40% B to 40% B in 18 min; Wave Length: 220/254 nm; RT1 (min): 9.83; RT2 (min): 13.99; The second peak in product; Sample Solvent: EtOH:DCM=1:1-HPLC; Injection Volume: 0.7 mL; Number of Runs: 6) to afford 581 (136.9 mg, 41.58%) as a yellow solid.

LC-MS-581: (ES, m/z): [M+H]⁺ 607

H-NMR-581: 1H NMR (400 MHz, DMSO-d6 ppm) δ 1.19-1.26 (m, 4H), δ 1.42-1.44 (d, 1H), δ 1.79-1.85 (m, 7H), δ 2.00-2.17 (m, 3H), δ 2.45-2.51 (d, 1H), δ 2.73-2.75 (d, 1H), δ 2.99-3.01 (d, 1H), δ 3.18-3.23 (m, 1H), δ 3.43 (s, 3H), δ 3.60 (s, 1H), δ 4.25-4.27 (d, 1H), δ 7.11 (s, 1H), δ 7.18-7.20 (d, 1H), δ 7.31 (s, 1H), δ 7.42-7.46 (t, 1H), δ 7.59 (s, 1H), δ 7.68-7.73 (m, 2H), δ 8.33 (s, 1H).

### Example 575. Synthesis of Compound 582

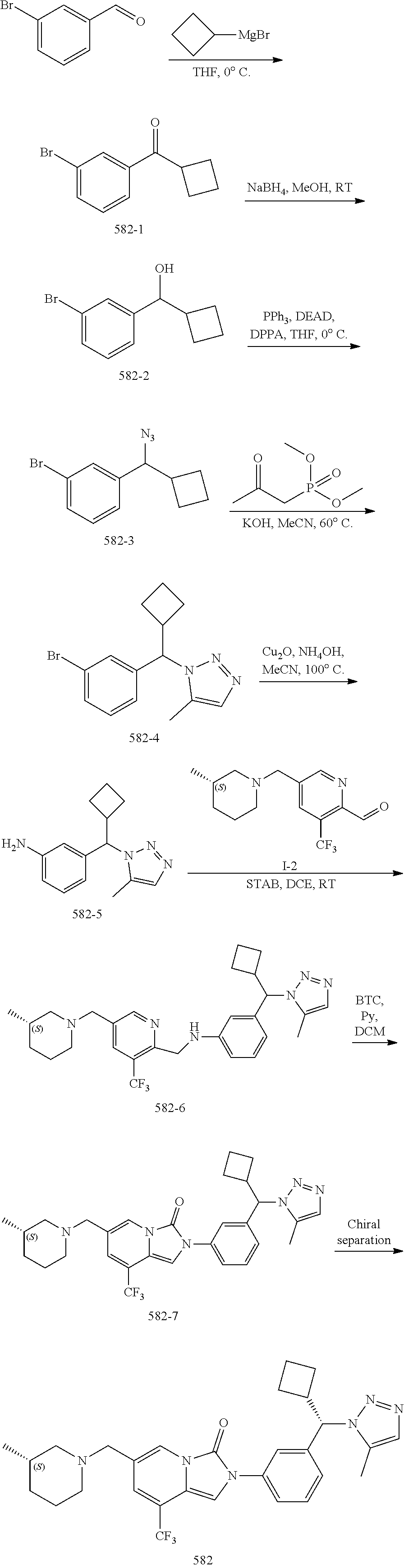

### 1. Synthesis of 582-1

To a stirred solution of 3-bromobenzaldehyde (45 g, 243.217 mmol, 1.00 equiv) in THF (500 mL) were added bromo(cyclobutyl)magnesium (486 mL, 486,434 mmol, 2 equiv) at 0° C. under nitrogen atmosphere. The resulting mixture was stirred for 1 h at 0° C. under nitrogen atmosphere. The reaction was quenched with saturated NH₄Cl (aq.) (2000 mL) at room temperature. The aqueous layer was extracted with EtOAc (2×500 mL). The resulting mixture was concentrated under reduced pressure. The residue was purified by silica gel column chromatography, elated with PE/EA=100:1 to afford 582-1 (9 g, 15.48%) as a yellow oil.

### 2. Synthesis of 582-2

To a stirred mixture of 582-1 (5 g, 20.911 mmol, 1 equiv) in MeOH (50 mL) were added NaBH₄ (1.58 g, 41.822 mmol, 2 equiv) at room temperature. The resulting mixture was stirred for 2 b at room temperature. The reaction was quenched with saturated NH₄Cl (aq.) (200 mL) at room temperature. The aqueous layer was extracted with EtOAc (2×50 mL). The resulting mixture was concentrated under reduced pressure. The residue was purified by Prep-TLC (PE/EA=1:1) to afford 582-2 (4 g, 79.33%) as a yellow oil.

### 3. Synthesis of 582-3

To a stirred solution of 582-2 (4 g, 16.589 mmol, 1 equiv) and DPPA (6.85 g, 24,883 mmol, 1.5 equiv) in THF (40 mL) was added DEAD (5.78 g, 33.178 mmol, 2 equiv) and PPh₃ (6.53 g, 24.883 mmol, 1.5 equiv) at room temperature under nitrogen atmosphere. The resulting mixture was stirred overnight at room temperature under nitrogen atmosphere. The reaction was quenched with water (200 mL) at room temperature. The aqueous layer was extracted with EtOAc (2×100 mL). The resulting mixture was concentrated under reduced pressure. The residue was purified by silica gel column chromatography, eluted with PE/EA=5:1 to afford 582-3 (3 g, 67.95%) as a yellow oil.

### 4. Synthesis of 582-4

To a stirred mixture of 582-3 (3 g, 11.272 mmol, 1 equiv) and dimethyl 2-oxopropylphosphonate (181 g, 16.908 mmol, 1.5 equiv) in MeCN (30 mL) were added KOH (1.90 g, 33,816 mmol, 3 equiv) at room temperature. The resulting mixture was stirred for 4 h at 80° C. The reaction was quenched with water (200 mL) at room temperature. The aqueous layer was extracted with EtOAc (2×50 mL). The resulting mixture was concentrated under reduced pressure. The residue was purified by Prep-TLC (PE/EA=1:1) to afford 582-4 (900 mg, 26.07%) as a yellow solid.

### 5. Synthesis of 582-5

To a stirred mixture of 582-4 (1.08 g, 3.527 mmol, 1.00 equiv) and NH₄0H (5.00 mL) in MeCN (5 mL) were added Cu2O (0.10 g, 0.705 mmol, 0.2 equiv) at room temperature. The resulting mixture was stirred overnight at 100° C. The resulting mixture was filtered, the filter cake was washed with MeOH (2×10 mL). The filtrate was concentrated under reduced pressure. The residue was purified by Prep-TLC (CH₂Cl₂/MeOH=30:1) to afford 582-5 (600 mg, 70.20%) as a yellow solid,

### 6. Synthesis of 582-6

To a stirred mixture of 582-5 (580 mg, 2.393 mmol, 1 equiv) and I-2 (822.29 mg, 2.872 mmol, 1.2 equiv) in DCE (6 mL) were added STAB (1521.82 mg, 7.179 mmol, 3 equiv) at room temperature. The resulting mixture was stirred overnight at room temperature. The reaction was quenched with saturated NaHCO₃ (aq.) (100 mL) at room temperature. The aqueous layer was extracted with CH₂Cl₂ (2×50 mL). The resulting mixture was concentrated under reduced pressure. The residue was purified by Prep-TLC (CH₂Cl₂/MeOH=20:1) to afford 582-6 (600 mg, 48.90%) as a yellow solid,

### 7. Synthesis of 582-7

To a stirred solution of 582-6 (580 mg, 1.131 mmol, 1 equiv) and pyridine (536.98 mg, 6.786 mmol, 6 equiv) in DCM (8 mL) were added Triphosgene (117.51 mg, 0.396 mmol, 0.35 equiv) at 0° C. The resulting mixture was stirred for 10 min at room temperature. The reaction was quenched with saturated NaHCO₃ (aq.) (80 mL) at room temperature. The aqueous layer was extracted with CH₂Cl₂ (2×50 mL). The resulting mixture was concentrated under reduced pressure. The residue was purified by Prep-TLC (CH₂Cl₂/MeOH=15:1) to afford 582-7 (450 mg, 73.84%) as a yellow solid.

### 8. Synthesis of 582

The 582-7 (450 mg) was purified by Prep-Chiral-HPLC with the following conditions (Column: CHIRALPAK IF, 2*25 cm, 5 μm; Mobile Phase A: Hex (0.5% 2 M NH₃-MeOH), Mobile Phase B: EtOH:DCM=1:1; Flow rate: 20 mL/min; Gradient: 25% B to 25% B in 10.5 min; Wave Length: 220/254 nm; RT1 (min): 5.97; RT2 (min): 8.25; the first peak is product) to afford 582 (201.8 ng, 44.62%) as a yellow solid.

LC-MS-582: (ES, m/z): [M+H]⁺ 539

H-NMR-582: (400 MHz, DMSO-d6, δ ppm): 0.82-0.93 (m, 4H), 1.40-1.49 (m, 1H), 1.49-1.84 (m, 4H), 1.84-1.86 (m, 1H), 1.86-1.98 (m, 6H), 2.24 (s, 3H), 2.72-2.77 (m, 2H), 3.24-3.30 (m, 2H), 3.53-3.59 (m, 1H), 5.60-5.63 (m, 1H), 7.01 (s, 1H), 7.26-7.28 (m, 1H), 7.28-7.32 (m, 1H), 7.47-7.51 (m, 2H), 7.65 (s, 1H), 7.74-7.76 (s, 1H), 7.82 (s, 1H).

### Example 576. Synthesis of Compound 583

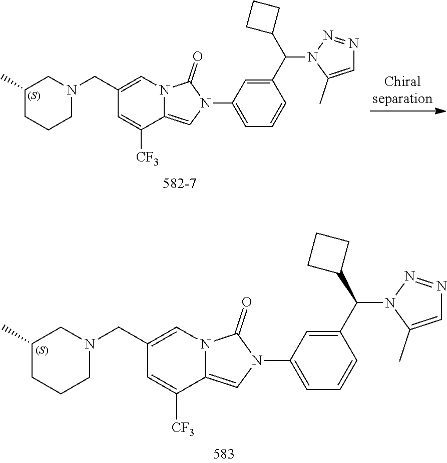

### 1. Synthesis of 583

The 582-7 (450 mg) was purified by Prep-Chiral-HPLC with the following conditions (Column: CHIRALPAK IF, 2*25 cm, 5 μm; Mobile Phase A: Hex (0.5% 2 M NH₃-MeOH), Mobile Phase B: EtOH:DCM=1:1; Flow rate: 20 mL/min; Gradient: 25% B to 25% B in 10.5 mii; Wave Length: 220/254 nm; RT1 (min): 5.97; RT2 (min): 8.25; the second peak is product) to afford 583 (201.8 mg, 43.71%) as a yellow solid.

LC-MS-583: (ES, m/z): [M+H]⁺ 539

H-NMR-583: (400 MHz, DMSO-d6, δ ppm): 0.82-0.93 (m, 4H), 1.40-1.49 (m, 1H), 1.49-1.84 (m, 4H), 1.84-1.86 (m, 1H), 1.86-1.97 (m, 6H), 2.24 (s, 3H), 2.72-2.77 (m, 2H), 3.24-3.30 (m, 2H), 3.53-3.63 (m, 1H), 5.60-5.63 (m, 1H), 7.01 (s, 1H), 7.26-7.28 (m, 1H), 7.28-7.32 (m, 1H), 7.47-7.51 (m, 2H), 7.65 (s, 1H), 7.74-7.76 (s, 1H), 7.85 (s, 1H).

### Example 577. Synthesis of Compound 584

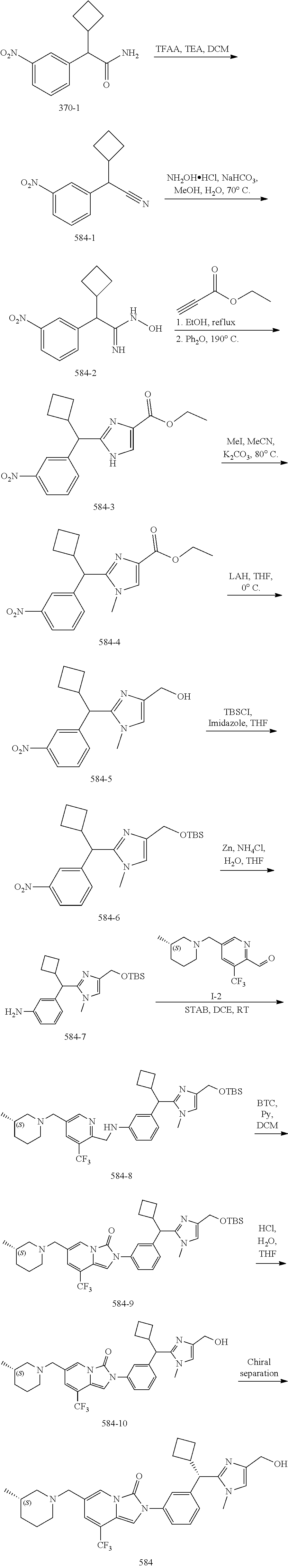

### 1. Synthesis of 584-1

To a stirred solution of 370-1 (39 g, 166.485 mmol, 1 equiv) and TEA (4112 g, 416.213 mmol, 2.5 equiv) in DCM (500 mL, 7865.301 mmol, 47.24 equiv) were added TFAA (41.96 g, 199,782 mmol, 1.2 equiv) dropwise at 0° C. under nitrogen atmosphere. The resulting mixture was stirred for 6 h at room temperature. The reaction was quenched with saturated NaHCO₃ (aq.) (1000 mL) at room temperature. The aqueous layer was extracted with EtOAc (3×300 mL). The combined organic layers were dried over anhydrous Na₂SO4. After filtration, the filtrate was concentrated under reduced pressure. The residue was purified by silica gel column chromatography, eluted with PE/EA (2:1) to afford 584-1 (32 g, 86.22%) as a light yellow oil.

### 2. Synthesis of 584-2

To a stirred solution of 584-1 (10 g, 46.245 mmol, 1 equiv) and NH₂OH·HCl (3.53 g, 50.870 mmol, 1.1 equiv) in MeOH (500 mL)/H2O (50 mL) were added NaHCO₃ (8.55 g, 101.739 mmol, 2.2 equiv) at room temperature. The resulting mixture was stirred overnight at 70° C. The resulting mixture was concentrated under vacuum. The residue was dissolved in DCM (500 mL) and dried over anhydrous Na₂SO₄. After filtration, the filtrate was concentrated under reduced pressure. This resulted in 584-2 (10 g, 69.40%) as a light yellow oil.

### 3. Synthesis of 584-3

To a stirred solution of 584-2 (10 g, 40.117 mmol, 1 equiv) in EtOH (200 mL) was added ethyl propiolate (5.90 g, 60,175 mmol, 1.5 equiv) at room temperature. The resulting mixture was stirred for overnight at 80° C. The resulting mixture was concentrated under vacuum. The residue was dissolved in phenoxybenzene (150 mL). The resulting mixture was stirred for 4 h at 190° C. The mixture was allowed to cool down to room temperature. The residue was purified by silica gel column chromatography, eluted with DCM/MeOH (50:1) to afford crude product. The residue was purified by Prep-TLC (PE/EA 2:1) to afford 584-3 (3.5 g, 23.84%) as a brown solid.

### 4. Synthesis of 584-4

To a stirred solution of 584-3 (3.5 g, 10,627 mmol, 1 equiv) and MeI (2.26 g, 15.941 mmol, 1.5 equiv) in MeCN (80 mL) was added K₂CO₃ (2.94 g, 21.254 mmol, 2 equiv) at room temperature. The resulting mixture was stirred overnight at room temperature. The resulting mixture was diluted with water (300 mL). The aqueous layer was extracted with EtOAc (2×200 mL). The resulting mixture was concentrated under vacuum. The residue was purified by Prep-TLC (PE/EA 3:1) to afford 584-4 (1.2 g, 30.58%) as a brown solid.

### 5. Synthesis of 584-5

To a stirred solution of 584-4 (1.2 g, 3,495 mmol, 1 equiv) in THF (50 mL) was added LAH (0.33 g, 8.738 mmol, 2.5 equiv) at 0° C. The resulting mixture was stirred for 4 h at room temperature. The reaction was quenched with saturated NH4Cl (aq.) (100 mL) at room temperature. The aqueous layer was extracted with DCM (3×100 ml). The combined organic layers were dried over anhydrous Na2SO4. After filtration, the filtrate was concentrated under reduced pressure. This resulted in 584-4 (1 g, 75.97%) as a brown yellow solid.

### 6. Synthesis of 584-6

To a stirred solution of 584-5 (1 g, 3.318 mmol, 1 equiv) and Imidazole (0.68 g, 9.954 mmol 3 equiv) in THF (50 mL) were added TBSCl (1.00 g, 6.635 mmol, 2.00 equiv) at room temperature. The resulting mixture was stirred for 3 h at 70° C. The reaction was quenched with saturated NH₄Cl (aq.) at room temperature. The aqueous layer was extracted with EtOAc (2×100 mL). The resulting mixture was concentrated under vacuum. This resulted in 584-6 (1 g, 58.01%) as a yellow oil. The crude product was used in the next step directly without further purification.

### 7. Synthesis of 584-7

To a stirred solution of 584-6 (1 g, 2.406 mmol, 1 equiv) and Zn (0.79 g, 12.030 mmol, 5 equiv) in THF (40 mL)/H₂O (10 mL) was added NH₄Cl (1.29 g, 24.060 mmol, 10 equiv) at room temperature. The resulting mixture was stirred overnight at room temperature. The resulting mixture was filtered, the filter cake was washed with EtOAc (2×10 mL). The resulting mixture was diluted with water (100 mL). The aqueous layer was extracted with EtOAc (3×60 mL). The resulting mixture was concentrated under vacuum. The residue was purified by Prep-TLC (DCM/MeOH 30:1) to afford 584-7 (620 mg, 60.14%) as a light yellow oil.

### 8. Synthesis of 584-8

To a stirred solution of 584-7 (600 mg, 1.556 mmol, 1 equiv) in DCE (10 mL) was added 1-2 (579.09 mg, 2.023 mmol, 1.3 equiv) at room temperature. The resulting mixture was stirred for 3 h at room temperature. To the above mixture was added STAB (659.52 mg, 3,112 mmol, 2 equiv) at room temperature. The resulting mixture was stirred for additional 3 h at room temperature. The reaction was quenched with saturated NHCO₃ (aq.) (80 mL) at room temperature. The aqueous layer was extracted with DCM (2×50 mL). The resulting mixture was concentrated under vacuum. The residue was purified by Prep-TLC (DCM/MeOH 30:1) to afford 584-8 (600 mg, 54.68%) as a light yellow oil.

### 9. Synthesis of 584-9

To a stirred solution of 584-8 (600 mg, 0,915 mmol, 1 equiv) and Pyridine (723.56 mg, 9.150 mmol, 10 equiv) in DCM (20 mL) were added Triphosgene (108.57 mg, 0.366 mmol, 0.4 equiv) at room temperature. The resulting mixture was stirred for 10 min at room temperature. The reaction was quenched with saturated NaHCO₃ (aq.) (80 mL) at room temperature. The aqueous layer was extracted with DCM (2×50 mL). The resulting mixture was concentrated under vacuum. This resulted in 584-9 (520 mg, 75.03%) as a yellow solid. The crude product was used in the next step directly without further purification.

### 10. Synthesis of 584-10

To a stirred solution of 584-9 (520 mg, 0,763 mmol, 1 equiv) in THF (10 mL) was added HCl (10 m L, 2M) at room temperature. The resulting mixture was stirred for 3 h at room temperature. The reaction was quenched with saturated NaHCO₃ (aq.) at room temperature. The aqueous layer was extracted with DCM (3×100 mL). The resulting mixture was concentrated under vacuum. The residue was purified by Prep-TLC (DCM/MeOH 20:1) to afford 584-10 (400 mg, 89.63%) as a yellow solid.

### 11. Synthesis of 584

The 584-10 (400 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/mi; Gradient: 20% B to 20% B in 17 min; Wave Length: 220/254 nm; RT1 (min): 11.78; RT2 (min): 14.16; the first peak is product) to afford 584 (116.1 mg, 28.85%) as a yellow solid.

LC-MS-584: (ES, m/z): [M+H]⁺ 568

H-NMR-584: (400 MHz, DMSO-d6, δ ppm): 0.81-0.95 (m, 4H), 1.41-1.55 (m, 1H), 1.55-1.71 (m, 5H), 1.71-1.82 (m, 4H), 1.82-1.98 (m, 1H), 2.02-2.06 (m, 1H), 2.73-2.77 (m, 2H), 3.14-3.25 (m, 3H), 3.44 (s, 3H), 4.12-4.14 (d, 1H), 4.30-4.32 (d, 2H), 4.72-4.75 (m, 1H), 6.81 (s, 1H), 7.01 (s, 1H), 7.21-7.23 (m, 1H), 7.28 (s, 1H), 7.39-7.43 (m, 1H), 7.64-7.66 (m, 2H), 7.76 (s, 1H).

### Example 578. Synthesis of Compound 585

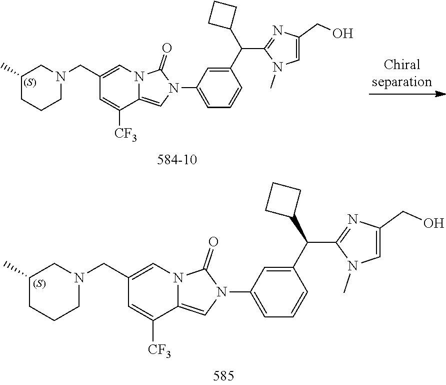

### 1. Synthesis of 585

The 584-10 (400 ng) was purified by Prep-Chiral-HPLC with the following conditions (Column: CHIRALPAK. IC, 2*25 cm, 5 μm; Mobile Phase A: Hex (0.5% 2M NH₃-MeOH), Mobile Phase B: EtOH:DCM=1:1; Flow rate: 20 mL/min; Gradient: 20% B to 20% B in 17 min; Wave Length: 220/254 m; RT1 (min): 11.78; RT2 (min): 14.16; the second peak is product) to afford 585 (102.9 mg, 25.62%) as a yellow solid.

LC-MS-585: (ES, m/z): [M+H]⁺ 568.

H-NMR-585: (400 MHz, DMSO-d6, δ ppm): 0.81-0.95 (m, 4H), 1.41-1.55 (m, 1H), 1.55-1.71 (m, 5H), 1.71-1.82 (m, 4H), 1.82-1.98 (m, 1H), 2.02-2.06 (m, 1H), 2.73-2.77 (m, 2H), 3.14-3.25 (m, 3H), 3.44 (s, 3H), 4.12-4.14 (d, 1H), 4.30-4.32 (d, 2H), 4.72-4.75 (m, 1H), 6.81 (s, 1H), 7.01 (s, 1H), 7.21-7.23 (m, 1H), 7.28 (s, 1H), 7.39-7.43 (m, 1H), 7.64-7.66 (m, 2H), 7.76 (s, 1H).

### Example 579. Synthesis of Compound 586

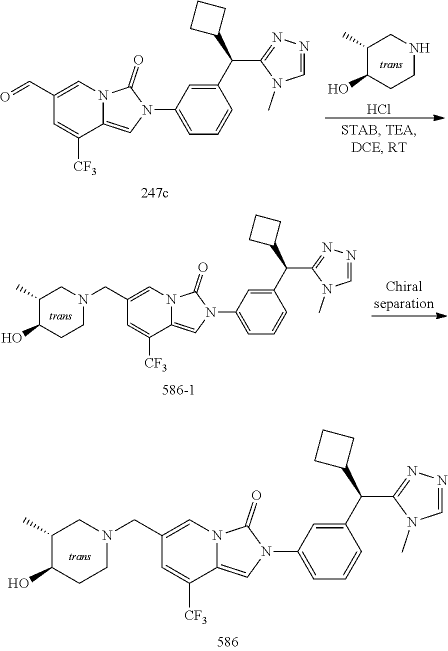

### 1. Synthesis of 586-1

To a stirred solution of 247c (1 g, 2.196 mmol, 1.00 equiv) and (3R,4R)-3-methylpiperidin-4-ol hydrochloride (0.50 g, 3.294 mmol, 1.5 equiv) in DCE (15 mL) were added TEA (0.44 g, 4.392 mmol 2 equiv) at room temperature. The resulting mixture was stirred for 1 h at room temperature. To the above mixture was added STAB (0.93 g, 4.392 mmol, 2 equiv) at room temperature. The resulting mixture was stirred overnight at room temperature. The reaction was quenched with saturated NH₄Cl (aq.) (30 mL) at room temperature. The resulting mixture was extracted with CH₂Cl₂ (3×50 mL). The resulting mixture was concentrated under reduced pressure. The crude product (600 mag) was purified by Prep-HPLC with the following conditions (Column: XBridge Prep OBD C18 Column, 19*250 mm, 5 μm; Mobile Phase A: Water (10 mmol/L NH₄HCO₃), Mobile Phase B: ACN; Flow rate: 60 mL/min; Gradient: 27% B to 47% B in 8 min, Wave Length: 254 nm; RT1 (min): 7.43) to afford 586-1 (400 mg, 32.19%) as a yellow solid.

### 2. Synthesis of 586

The 586-1 (400 mg) was purified by Chiral separation with the following conditions (Column: CHIRALPAK IC, 2*25 cm, 5 man; Mobile Phase A: Hex (0.5% 2M NH₃-MeOH), Mobile Phase B: EtOH:DCM=1:1; Flow rate: 20 mL/min; Gradient: 70% B to 70% B in 10.5 min; Wave Length: 220/254 nm; RT1 (min): 6.32; RT2 (min): 8.41; the first peak is product) to afford 586 (49.9 mg, 12.34%) as a yellow solid.

LC-MS-586: (ES, m/z): [M+H]⁺ 555

H-NMR-586: (400 MHz, DMSO-d6, δ ppm): 0.81-0.92 (d, 3H), 1.31-1.51 (m, 2H), 1.61-1.87 (m, 7H), 1.92-2.12 (m, 2H), 2.71-2.83 (m, 2H), 2.89-2.98 (m, 1H), 3.19-3.25 (m, 3H), 3.43 (s, 3H), 4.25-4.27 (d, 1H), 4.53-4.55 (d, 1H), 7.00 (s, 1H), 7.18-7.20 (d, 1H), 7.31 (s, 1H), 7.42-7.46 (m, 1H), 7.64-7.74 (m, 3H), 8.34 (s, 1H).

### Example 580. Synthesis of Compound 587

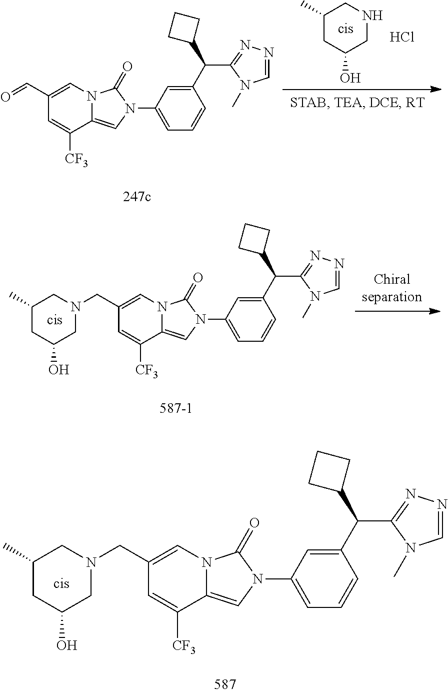

### 1. Synthesis of 587-1

To a stirred solution of 247c (2.5 g, 5.4945 mmol, 1 equiv) and (3R,5S)-5-methylpiperidin-3-ol (0.95 g, 8.233 mmol, 1.5 equiv) in DCE (25 mL) was added TEA (1.11 g, 10.978 mmol, 2 equiv) at room temperature. The resulting mixture was stirred for 1 h at room temperature. To the above mixture was added STAB (2.33 g, 10.978 mmol, 2 equiv) at room temperature. The resulting mixture was stirred for additional 5 h at room temperature. The mixture was quenched with saturated NaHCO₃ (aq.) (200 mL). The aqueous layer was extracted with DCM (3×100 mL). The resulting mixture was concentrated under reduced pressure. The residue was purified by reverse flash chromatography with the following conditions: column, C18 silica gel; mobile phase, MeCN in Water (10 mmol/L NH4HCO₃), 30% to 60% gradient in 20 min; detector, UV 254 nm. This resulted in 587-1 (1 g, 31.20%) as a yellow solid.

### 2. Synthesis of 587-2

The 587-1 (300 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 ml/min; Gradient: 40% B to 40% B in 16 min; Wave Length: 220/254 nm; RT1 (min): 8.12; RT2 (min): 12.20; the first peak is product) to afford 587 (77.9 mg, 25.81%) as a yellow solid,

LC-MS: (ES, m/z): [M+H]⁺ 555. H-NMR: (400 MHz, DMSO-d6, δ ppm): 0.67-0.81 (m, 1H), 0.81-0.94 (d, 3H), 1.88-1.92 (m, 9H), 2.06-2.10 (m, 1H), 2.68-2.73 (m, 1H), 2.67-2.73 (m, 1H), 2.81-2.93 (m, 1H), 3.19-3.30 (m, 2H), 3.30-3.32 (m, 1H), 3.36-3.48 (m, 4H), 4.25-4.28 (d, 1H), 4.61-4.71 (m, 1H), 7.01 (s, 1H), 7.19-7.21 (d, 1H), 7.32 (s, 1H), 7.44-7.46 (m, 1H), 7.68-7.74 (m, 3H), 8.33 (s, 1H).

### Example 581. Synthesis of Compound 588

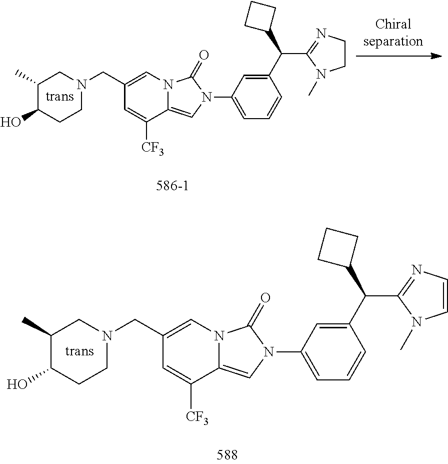 [SMILES: C[C@@H]1CN(Cc2cc(C(F)(F)F)c3cn(-c4cccc([C@H](C5=NCCN5C)C5CCC5)c4)c(=O)n3c2)CC[C@H]1O ; C[C@H]1CN(Cc2cc(C(F)(F)F)c3cn(-c4cccc([C@H](c5nccn5C)C5CCC5)c4)c(=O)n3c2)CC[C@@H]1O]

### 1. Synthesis of 588-0

The 586-1 (400 mg) was purified by Chiral separation with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 70% B to 70% B in 10.5 min; Wave Length: 220/254 nm; RT1 (min): 6.32; RT2 (min): 8.41; the second peak is product) to afford 588 (54.0 mg, 13.14%) as a yellow solid.

LC-MS: (ES, m/z): [M+H]+ 555. H-NMR: (400 MHz, DMSO-d6, δ ppm): 0.81-0.92 (d, 3H), 1.31-1.51 (m, 2H), 1.61-1.87 (m, 7H), 1.92-2.12 (m, 2H), 2.72-2.83 (m, 2H), 2.90-2.98 (m, 1H), 3.19-3.24 (m, 3H), 3.43 (s, 3H), 4.25-4.27 (d, 1H), 4.53-4.55 (d, 1H), 7.00 (s, 1H), 7.18-7.20 (d, 1H), 7.31 (s, 1H), 7.42-7.46 (m, 1H), 7.64-7.74 (m, 3H), 8.34 (s, 1H).

### Example 582. Synthesis of Compound 589

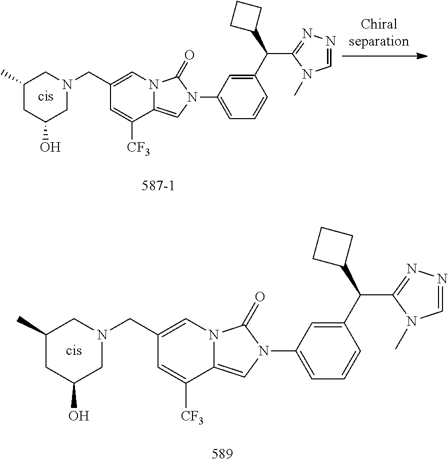 [SMILES: C[C@H]1C[C@@H](O)CN(Cc2cc(C(F)(F)F)c3cn(-c4cccc([C@H](c5nncn5C)C5CCC5)c4)c(=O)n3c2)C1 ; C[C@@H]1C[C@H](O)CN(Cc2cc(C(F)(F)F)c3cn(-c4cccc([C@H](c5nncn5C)C5CCC5)c4)c(=O)n3c2)C1]

### 1. Synthesis of 589

The 587-1 (300 mg) was purified by Prep-Chiral-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 16 min; Wave Length: 220/254 nm; RT1 (min): 8.12; RT2 (min): 12.20; the second peak is product) to afford 589 (90.7 mg, 30.11%) as a yellow solid.

LC-MS-589: (ES, m/z): [M+H]⁺ 555

H-NMR-589: (400 MHz, DMSO-d6, δ ppm): 0.67-0.81 (m, 1H), 0.81-0.94 (d, 3H), 1.48-1.68 (m, 3H), 1.68-1.92 (m, 6H), 2.06-2.10 (m, 1H), 2.68-2.73 (m, 1H), 2.67-2.73 (m, 1H), 2.81-2.93 (m, 1H), 3.19-3.30 (m, 2H), 3.30-3.32 (m, 1H), 3.36-3.48 (m, 4H), 4.25-4.28 (d, 1H), 4.61-4.71 (m, 1H), 7.00 (s, 1H), 7.18-7.20 (d, 1H), 7.31 (s, 1H), 7.44-7.46 (m, 1H), 7.67-7.74 (m, 3H), 8.33 (s, 1H).

### Example 583. Synthesis of Compound 590

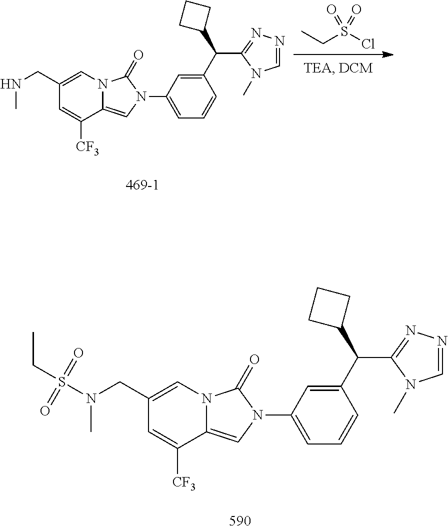

### 1. Synthesis of 590

Into a 20 mL sealed tube were added 469-1 (300 mg, 0.638 mmol, 1 equiv), DCM (4 mL, 62.922 mmol, 98.68 equiv), TEA (193.57 mg, 1.914 mmol, 3 equiv) and ethanesulfonyl chloride (81.98 mg, 0.638 mmol, 1 equiv) at room temperature. The solution was stirred for 1.5 h at room temperature. The reaction was quenched with sat. NaHCO₃ (aq.) (20 mL) at room temperature. The aqueous layer was extracted with CH₂Cl₂ (3×10 mL). The resulting mixture was concentrated under reduced pressure. The crude product (160 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 52% B in 8 min; Wave Length: 254 nm; RT1 (min): 7.47) to afford 590 (93.4 mg, 25.67%) as a yellow solid.

LC-MS-590: (ES, m/z): [M+H]⁺ 563

H-NMR-590: (400 MHz, DMSO, δ ppm): 1.15-1.33 (m, 3H), 1.68-1.91 (m, 3H), 2.00-2.18 (s, 1H), 2.71-2.80 (s, 3H), 3.16-3.30 (m, 3H), 3.39-3.52 (s, 3H), 4.08-4.28 (s, 2H), 4.28-4.43 (d, 1H), 6.88-7.00 (s, 1H), 7.17-7.26 (m, 1H), 7.32-7.41 (s, 1H), 7.41-7.52 (m, 1H), 7.63-7.79 (m, 2H), 7.79-7.88 (m, 1H), 8.39-8.51 (s, 1H).

### Example 584. Synthesis of Compound 591

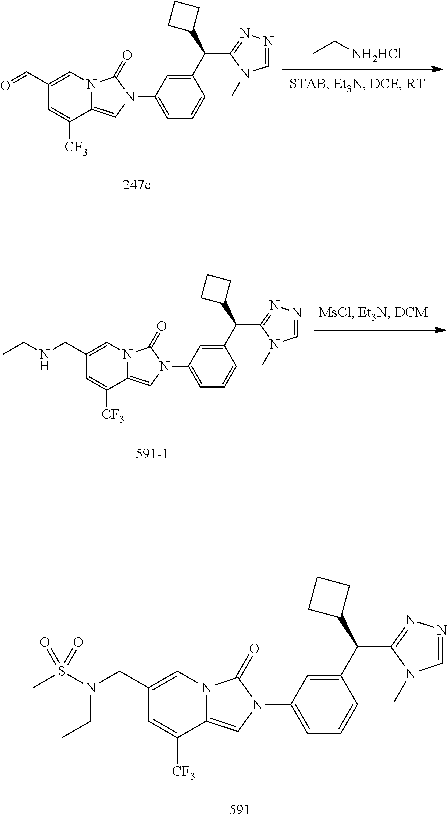

### 1. Synthesis of 591-1

A solution of 247c (2 g, 4.39 mmol, 1.0 equiv), ethylamine, hydrochloride (1.1 g, 13.17 mmol, 3.0 equiv) and TEA (1.3 g, 13.17 mmol, 3.0 equiv) in DCE (20 mL) was stirred for 30 min at room temperature. To the above mixture was added STAB (1.9 g, 8.78 mmol, 2.0 equiv) at room temperature. The resulting mixture was stirred for additional overnight at room temperature. The reaction was quenched by the addition of sat. NH₄Cl (aq.) (60 mL) at room temperature. The aqueous layer was extracted with CH₂Cl₂ (3×20 mL). The resulting mixture was concentrated under reduced pressure. The residue was purified by Prep-TLC (CH₂Cl₂/MeOH=10:1) to afford 591-1 (600 mg, 25%) as a yellow solid.

### 2. Synthesis of 591

To a stirred solution of 591-1 (300 mg, 0.62 mmol, 1.0 equiv) and TEA (188 mg, 1.86 mmol, 3.0 equiv) in DCM (3 mL) was added MsCl (71 mg, 0.62 mmol, 1.0 equiv) at room temperature. The resulting mixture was stirred for additional 6 h at room temperature. The reaction was quenched by the addition of water (20 mL) at room temperature. The aqueous layer was extracted with CH₂Cl₂ (3×5 mL). The resulting mixture was concentrated under reduced pressure. The residue was purified by Prep-TLC (CH₂Cl₂/MeOH=15:1) to afford 591 (100 mg, 28%) as a yellow solid.

LC-MS-591: (ES, m/z): [M+H]⁺ 563

H-NMR-591: (400 MHz, DMSO-d6, δ ppm): 1.00-1.20 (t, 3H), 1.81-1.86 (m, 5H), 2.08-2.10 (m, 1H), 3.02 (s, 3H), 3.18-3.28 (m, 3H), 3.43 (s, 3H), 4.16 (s, 2H), 4.25-4.27 (d, 2H), 7.03 (s, 1H), 7.19-7.21 (d, 1H), 7.32 (s, 1H), 7.43-7.47 (t, 1H), 7.69-7.73 (m, 2H), 7.89 (s, 1H), 8.33 (s, 1H).

### Example 585. Synthesis of Compound 592

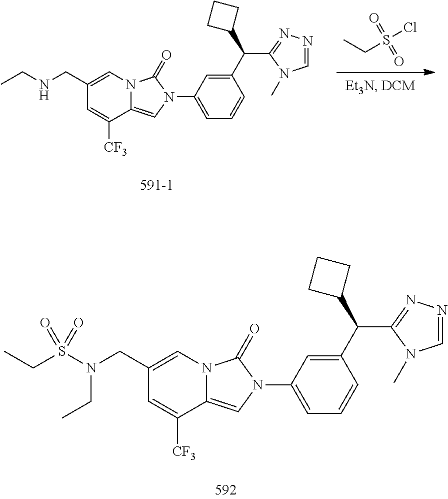

### 1. Synthesis of 592

To a stirred solution of 591-1 (300 mg, 0.62 mmol, 1.0 equiv) and TEA (188 mg, 1.86 mmol, 3.0 equiv) in DCM (3 mL) were added ethanesulfonyl chloride (80 mg, 0.62 mmol, 1.0 equiv) at room temperature. The resulting mixture was stirred for overnight at room temperature. The reaction was quenched by the addition of Water (20 mL) at room temperature. The aqueous layer was extracted with Cl₂Cl₂ (3×5 mL). The resulting mixture was concentrated under reduced pressure. The residue was purified by Prep-TLC (Cl₂Cl₂/MeOH=15:1) to afford crude product. The crude product (130 mg) was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 30 mL/min; Gradient: 41% B to 51% B in 8 min, 51% B; Wave Length: 254; 220 nm; RT1 (min): 7.98) to afford 592 (23 mg, 6%) as a yellow solid.

LC-MS-592: (ES, m/z): [M+H]⁺ 577

H-NMR-592: (400 MHz, DMSO-d6, δ ppm): 1.00-1.15 (t, 3H), 1.22-1.25 (m, 3H), 1.68-1.81 (m, 5H), 2.09-2.10 (m, 1H), 3.16-3.25 (m, 5H), 3.43 (s, 3H), 4.21 (s, 2H), 4.25-4.28 (d, 1H), 7.04 (s, 1H), 7.19-7.21 (d, 1H), 7.36 (s, 1H), 7.43-7.47 (t, 1H), 7.69-7.71 (m, 1H), 7.74 (s, 1H), 7.88 (s, 1H), 8.35 (s, 1H).

### Example 586. Synthesis of Compound 593

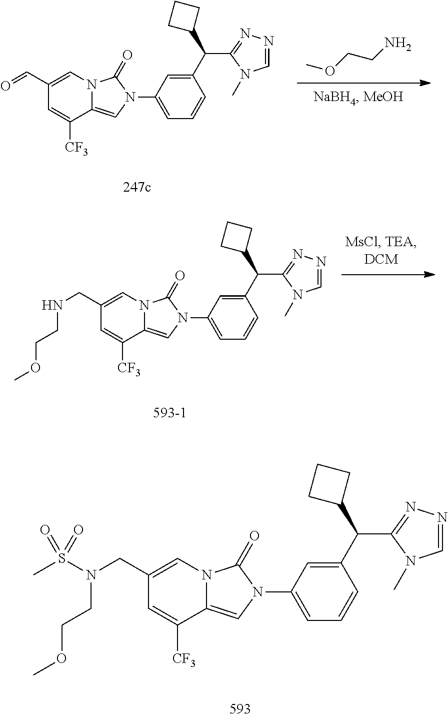

### 1. Synthesis of 593-1

Into a 100 mL 3-necked round-bottom flask were added 247c (500 mg, 1.098 mmol, 1 equiv), ethanamine, 2-methoxy- (82.46 mg, 1.098 mmol, 1 equiv) and MeOH (10 mL) at room temperature. The resulting mixture was stirred for overnight at room temperature. To the above mixture was added NaBH₄ (83.06 mg, 2.196 mmol, 2 equiv) at 0° C. The resulting mixture was stirred for additional 2 h at room temperature. The reaction was quenched by the addition of sat. NH4Cl (aq.) (20 mL) at room temperature. The aqueous layer was extracted with EtOAc (3×15 mL). The resulting mixture was concentrated under reduced pressure. The residue was purified by Prep-TLC, eluted with CH₂Cl₂/MeOH (12:1) to afford 593-1 (210 mg, 37.18%) as a light yellow solid,

### 2. Synthesis of 593

Into a 100 mL round-bottom flask were added 593-1 (200 mg, 0.389 mmol, 1 equiv), DCM (5.00 mL), TEA (118.00 mg, 1.167 mmol, 3 equiv) and MsCl (53.42 mg, 0.467 mmol, 1.2 equiv) at room temperature. The resulting mixture was stirred for 2 h at room temperature. The reaction was quenched by the addition of water (10 mL) at room temperature. The aqueous layer was extracted with CH₂Cl₂ (3×10 mL). The resulting mixture was concentrated under reduced pressure. The residue was purified by Prep-TLC, eluted with CH₂Cl₂/MeOH (10:1) to afford crude product (150 mg). The crude product (150 mg) was purified by Prep-HPLC with the following conditions (Column: XBridge Prep OBD C18 Column, 30*150 m, 5 μm; Mobile Phase A: Water (10 mmol/L NH₄HCO₃), Mobile Phase B: ACN; Flow rate: 60 mL/min; Gradient: 25% B to 55% B in 7 min; Wave Length: 220 urn; RT1 (min): 770) to afford 593 (73.0 mg, 31.69%) as a light yellow solid.

LC-MS: (ES, m/z): [M+H]⁺ 593. H-NMR: 1H NMR (300 MHz, DMSO-d6 ppm) δ 1.62-1.90 (m, 5H), 2.1 (s, 1H), 3.05 (s, 3H), 3.19 (s, 4H), 3.31-3.35 (m, 2H), 3.36-3.47 (m, 5H), 4.19 (s, 2H), 4.25-4.27 (d, 1H), 7.05 (s, 1H), 7.19-7.21 (d, 1H), 7.34 (s, 1H), 7.42-7.46 (m, 1H), δ 7.69-7.74 (m, 1H), 7.86 (s, 1H), 8.32 (s, 1H).

### Example 587. Synthesis of Compound 594

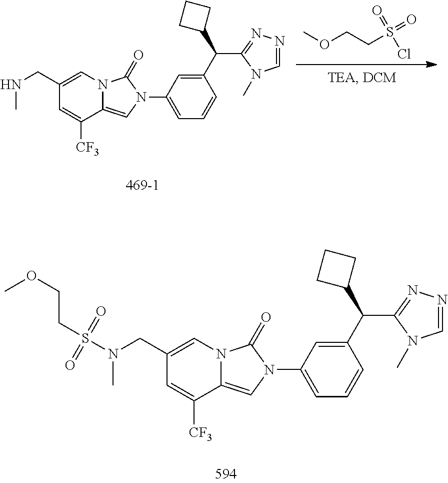

### 1. Synthesis of 594

Into a 20 mL sealed tube were added 469-1 (300 mg, 0.614 mmol, 1 equiv), DCM (4 mL, 62.922 mmol, 102.49 equiv), TEA (186.38 mg, 1.842 mmol, 3 equiv) and 2-methoxyethanesulfonyl chloride (97.37 mg, 0.614 mmol, 1 equiv) at room temperature. The mixture was stirred for 1.5 h at room temperature. The reaction was quenched with sat. NaHCO₃ (aq.) (20 mL) at room temperature. The aqueous layer was extracted with CH₂Cl₂ (3×10 mL). The resulting mixture was concentrated under reduced pressure. The residue was purified by Prep-TLC (CH₂Cl₂/MeOH 15:1) to afford crude product. The crude product (180 mg) was purified by Prep-HPLC with the following conditions (Column: XBridge Prep OBD C18 Column, 3*150 mm, 5 μm; Mobile Phase A: Water (10 mmol/L NH₄HCO3), Mobile Phase B: ACN; Flow rate: 60 mL/min; Gradient: 32% B to 51% B in 8 min; Wave Length: 254 nm; RT1 (min): 7.47) to afford 594 (112 mg, 30.57%) as a yellow solid,

LC-MS: (ES, m/z): [M+H]⁺ 593. H-NMR: (400 MHz, DMSO, δ ppm): 1.52-1.97 (m, 5H), 2.03-2.19 (s, 1H), 2.70-2.81 (s, 3H), 3.17-3.28 (s, 1H), 3.30-3.35 (s, 3H), 3.41-3.50 (m, 5H), 3.67-3.72 (m, 2H), 4.00-4.15 (s, 2H), 4.20-4.40 (d, 1H), 6.96-7.01 (s, 1H), 7.18-7.23 (m, 1H), 7.35-7.41 (s, 1H), 7.41-7.50 (m, 1H), 7.65-7.78 (m, 2H), 7.82-7.91 (s, 1H), 8.38-8.46 (s, 1H).

### Example 588. Synthesis of Compound 595

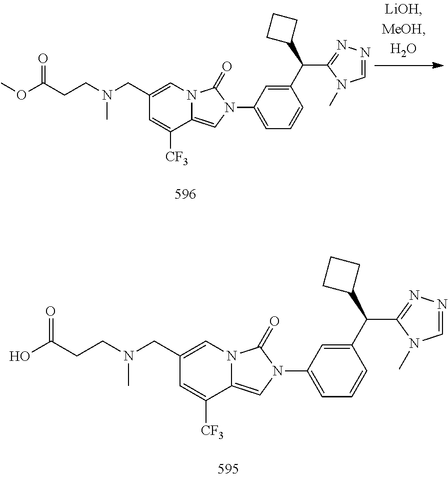 [SMILES: COC(=O)CCN(C)Cc1cc(C(F)(F)F)c2cn(-c3cccc([C@H](c4nncn4C)C4CCC4)c3)c(=O)n2c1 ; CN(CCC(=O)O)Cc1cc(C(F)(F)F)c2cn(-c3cccc([C@H](c4nncn4C)C4CCC4)c3)c(=O)n2c1]

### 1. Synthesis of 595

Into a 100 mL round-bottom flask were added 596 (250 ng, 0.449 mmol, 1 equiv), MeOH (6.00 mL), H₂O (1.5 mL) and LiOH (53.79 mg, 2,245 mmol, 5 equiv) at room temperature. The resulting mixture was stirred for overnight at room temperature. The residue was acidified to pH 6 with 1 M HCl (aq.) (2 ml). The reaction was quenched by the addition of water (10 mL) at room temperature. The aqueous layer was extracted with EtOAc (3×10 ml). The resulting mixture was concentrated under reduced pressure. The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 15% B to 35% B in 8 min; Wave Length: 254 nm; RT1 (min): 7.27) to afford 595 (85.9 mg, 35.25%) as a light yellow solid.

LC-MS-595: (ES, m/z): [M+H]⁺ 543

H-NMR-595: 1H NMR (300 MHz, DMSO-d6 ppm) δ 1.60-1.90 (m, 6H), 2.10 (s, 1H), 2.16 (s, 3H), 2.33-2.42 (m, 2H), 2.63-2.68 (m, 2H), 3.19-3.23 (m, 1H), 3.30 (s, 1H), 3.43 (s, 3H), 4.25-4.27 (d, 1H), 7.00 (s, 1H), 7.18-7.20 (d, 1H), 7.31 (s, 1H), 7.42-7.46 (m, 1H), δ 7.69-7.71 (m, 2H), 7.74 (s, 1H), 8.33 (s, 1H).

### Example 589. Synthesis of Compound 596

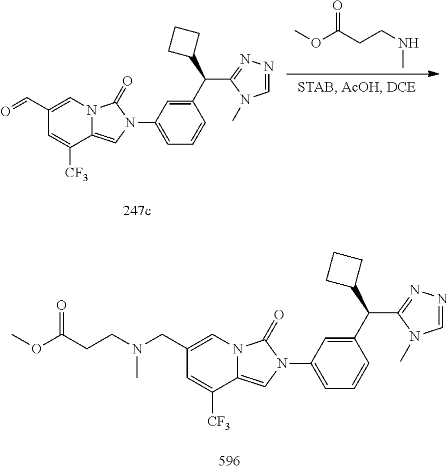

### 1. Synthesis of 596

Into a 100 mL round-bottom flask were added 247c (500 mg, 1.098 mmol, 1 equiv), DCE (7 mL), methyl 3-(methylamino)propanoate (257.22 mg, 2.196 mmol, 2 equiv), STAB (465.35 mg, 2.196 mmol, 2 equiv) and AcOH (65.93 mg, 1.098 mmol, 1 equiv) at room temperature. The resulting mixture was stirred for 4 h at room temperature. The reaction was quenched by the addition of sat. NH₄Cl (aq.) (10 mL) at room temperature. The aqueous layer was extracted with CH₂Cl₂ (3×10 mL). The resulting mixture was concentrated under reduced pressure. The crude product (400 mg) was purified by Prep-HPLC with the following conditions (Column: XBridge Prep OBD C18 Column, 3*150 mm, 5 μm; Mobile Phase A: Water (10 mmol/L NH₄HCO₃), Mobile Phase B: ACN; Flow rate: 60 mL/min; Gradient: 35% B to 53% B in 8 min; Wave Length: 254 nm; RT1 (min): 7.53) to afford 596 (300 mg, 49.10%) as a light yellow solid.

LC-MS: (ES, m/z): [M+H]⁺ 557. H-NMR: 1H NMR (300 MHz, DMSO-d6 ppm) δ 1.60-1.90 (m, 6H), 2.10 (s, 1H), 2.16 (s, 3H), 2.63-2.68 (m, 2H), 3.19-3.23 (m, 1H), 3.30 (s, 1H), 3.43 (s, 3H), 3.59 (s, 3H), 4.25-4.27 (d, 1H), 6.95 (s, 1H), 7.18-7.20 (d, 1H), 7.31 (s, 1H), 7.42-7.46 (m, 1H), 7.68-7.74 (m, 3H), 8.33 (s, 1H).

### Example 590. Synthesis of Compound 597_P1&P2

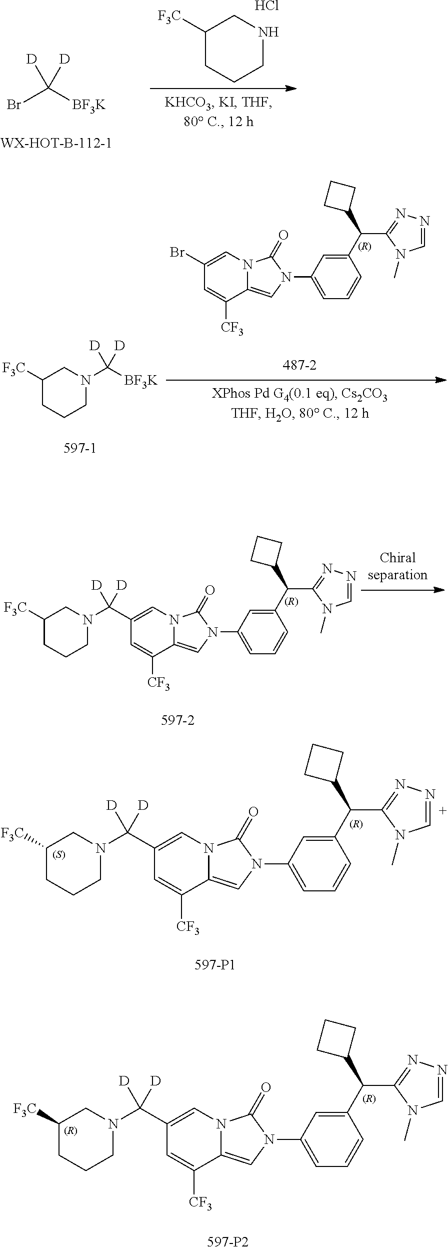

### 1. Synthesis of 597-1

To a solution of 527-1 (500 mg, 2.64 mmol, 1 equiv) in THF (20 mL) was added [bromo(dideuterio)methyl]-trifluoro-boron;potassium hydride (534 mg, 2.64 mmol, 1 equiv), KHCO₃ (528 mg, 5.27 mmol, 2 equiv), KI (43.78 mg, 263 umol, 0.1 equiv). Then the mixture was heated to 80° C. and stirred for 12 h under N2 atmosphere. The mixture was concentrated in vacuum to get a residue. The residue was dissolved with acetone (50 mL). Then the mixture was heated to 50° C. and stirred for 3 h. The mixture was filtered, the filtrate was concentrated and get 597-1 as a light yellow solid.

### 2. Synthesis of 597-2

To a solution of 487-2 (400 mg, 790 umol, 1 equiv) in THF (10 mL) and H₂O (2.5 mL) was added 597-1 (869 mg, 3.16 mmol, 4 equiv), Cs₂CO₃ (772 mg, 2.37 mmol, 3 equiv), dicyclohexyl-[2-(2,4,6-triisopropylphenyl)phenyl]phosphane;methanesulfonate;[2-[2-(methylamino)phenyl]phenyl]palladium(1+) (67.98 mg, 79.00 umol, 0.1 equiv) under N₂. The suspension was degassed and purged with N₂ for 3 tines. The mixture was heated to 80° C. and stirred for 12 h under N2. The mixture was poured into H2O (20 mL), extracted with EtOAc (20 ml×3). The combined organic layers were washed with brine (20 mL×2), dried over Na₂SO₄, filtered and concentrated under reduced pressure to give a residue. The residue was purified by column (SiO₂) with petroleum ether/EtOAc=2:1 to 0:1, then with DCM:MeOH=1:0 to 1:1 to obtain 597-2,

¹H-NMR-597-2: (400 MHz, CDCl₃) δ 8.03 (s, 1H), 7.60-7.53 (m, 3H), 7.41-7.39 (m, 1H), 7.18-7.16 (m, 1H), 6.86 (s, 1H), 6.76 (s, 1H), 4.10-4.08 (m, 1H), 3.98-3.95 (m, 1H), 3.54-3.50 (m, 3H), 3.43 (s, 3H), 3.25-3.23 (m, 1H), 2.34-2.32 (m, 1H), 1.94-1.80 (m, 10H).

### 3. Synthesis of 597-P1&P2

597-2 (280 ng) was purified by Chiral separation with the following conditions (column: column: DAICEL CHIRALPAK IG (250 mm*30 mm, 10 um);mobile phase: [Heptane-EtOH]; B %: 40%-80%, 15 nm, Flow rate: 40 mL/min; Wave Length: 220/254 nm; RT1 (min): 5.01; RT2 (min): 6.00) to afford crude 597-PI and crude 597-PI. The crude 597-P1 (90 mg) was further purified by column (SiO₂) with DCM:MeOH=1:0 to 10:1 to obtain 597-P1 (53 mg 1.8.5%) as a yellow solid.

MS-597_P1: (ES, m/z): [M+H]⁺ 595.2.

¹H-NMR-597_P1: (400 MHz, DMSO) δ 8.32 (s, 1H), 7.73-7.67 (m, 3H), 7.45-7.41 (m, 1H), 7.30 (s, 1H), 7.19-7.17 (m, 1H), 7.01-7.00 (m, 1H), 4.26-4.24 (m, 1H), 3.42 (s, 3H), 3.24-3.20 (m, 1H), 2.98-2.97 (m, 1H), 2.77-2.76 (m, 1H), 2.12-2.10 (m, 1H), 2.00-1.95 (m, 2H), 1.83-1.71 (m, 8H), 1.52-1.50 (m, 1H), 1.25-1.23 (m, 1H).

The crude 597-P2 (110 mg) was further purified by Prep-HPLC with the following conditions (Column: Phenomenex Luna C18 75*30 mm*3 um; mobile phase: [water (PA)-ACN]; B %: 1%-50%, 8 min; Wave Length: 220 nm; RT1 (min): 7.0) to afford 597_P2 (45 mg, 15.5%) as a yellow solid.

MS-597_P2: (ES, m/z): [M+H]⁺ 595.2.

¹H-NMR-597_P2: (400 MHz, DMSO) δ 8.32 (s, 1H), 7.73-7.72 (m, 1H), 7.71-7.69 (m, 1H), 7.68-7.67 (m, 1H), 7.45-7.41 (m, 1H), 7.30 (s, 1H), 7.19-7.17 (m, 1H), 7.01-7.00 (m, 1H), 4.26-4.24 (m, 1H), 3.42 (s, 3H), 3.24-3.20 (m, 1H), 2.98-2.97 (m, 1H), 2.77-2.76 (m, 1H), 2.12-2.10 (m, 1H), 2.00-1.95 (m, 2H), 1.84-1.80 (m, 5H), 1.76-1.71 (m, 3H), 1.52-1.50 (m, 1H), 1.25-1.23 (m, 1H).

### Example 591. Synthesis of Compound 598

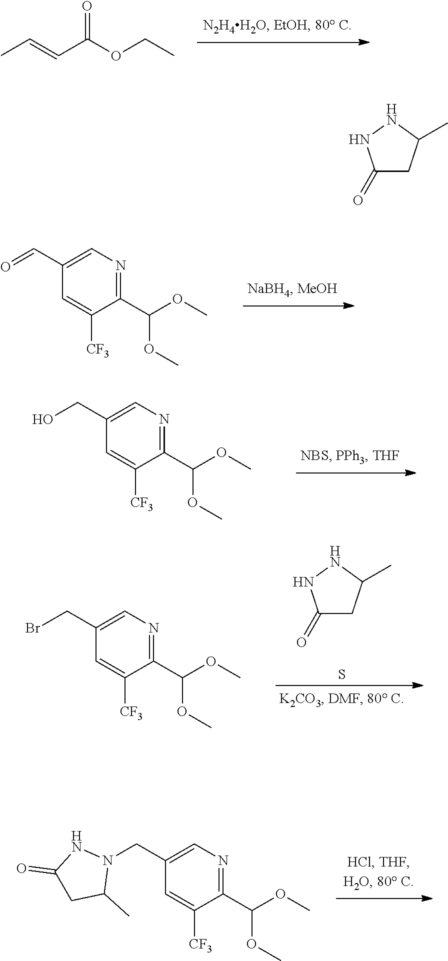

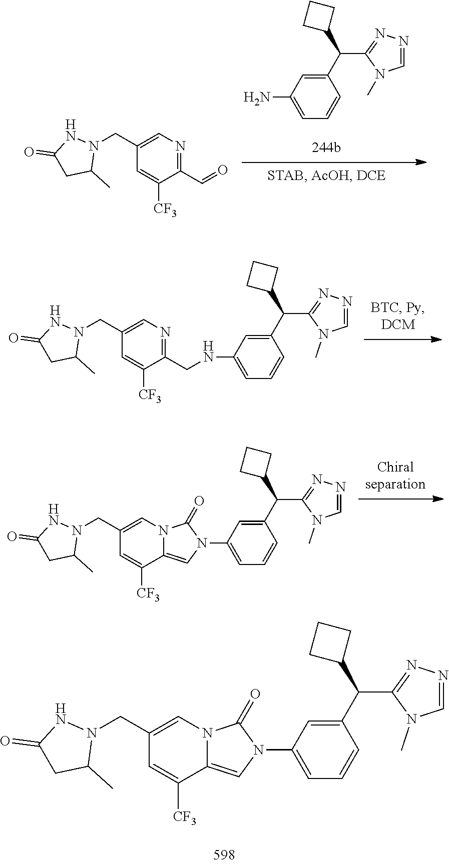 [SMILES: Cn1cnnc1[C@@H](c1cccc(N)c1)C1CCC1 ; CC1CC(=O)NN1Cc1cnc(C=O)c(C(F)(F)F)c1 ; CC1CC(=O)NN1Cc1cnc(CNc2cccc([C@H](c3nncn3C)C3CCC3)c2)c(C(F)(F)F)c1 ; CC1CC(=O)NN1Cc1cc(C(F)(F)F)c2cn(-c3cccc([C@H](c4nncn4C)C4CCC4)c3)c(=O)n2c1 ; CC1CC(=O)NN1Cc1cc(C(F)(F)F)c2cn(-c3cccc([C@H](c4nncn4C)C4CCC4)c3)c(=O)n2c1]

### 1. Synthesis of 598-1

Into a 500 ml, round-bottom flask were added ethyl crotonate (15 g, 131.413 mmol, 1 equiv), EtOH (150 mL) and hydrazine hydrate (7.89 g, 157.696 mmol, 1.2 equiv) at room temperature. The resulting mixture was stirred for 2 h at 80° C. The resulting mixture was concentrated under reduced pressure. This resulted in 598-1 (12 g, 91.25%) as a yellow oil.

### 2. Synthesis of 598-2

Into a 250 mL round-bottom flask were added 6-(dimethoxymethyl)-5-(trifluoromethyl)pyridine-3-carbaldehyde (5 g, 20.065 mmol, 1 equiv), methanol (50 mL) and NaBH4 (0.38 g, 10,033 mmol, 0.5 equiv) at room temperature. The resulting mixture was stirred for 20 min at room temperature. The reaction was quenched with sat. NH₄Cl (50 mL) at room temperature. The resulting mixture was extracted with CH₂Cl₂ (3×100 mL). The resulting mixture was concentrated under reduced pressure. The residue was purified by silica gel column chromatography, eluted with PE/EtOAc (10:1) to afford 598-2 (3 g, 56.54%) as a yellow oil.

### 3. Synthesis of 598-3

Into a 250 mL round-bottom flask were added 598-2 (3 g, 11.942 mmol, 1 equiv), THF (30 mL), PPh₃ (4.70 g, 17.913 mmol, 1.5 equiv) and NBS (3.19 g, 17.913 mmol, 1.5 equiv) at 0° C. The resulting mixture was stirred for 3 h at room temperature. The reaction was quenched by the addition of water (30 mL) at room temperature. The aqueous layer was extracted with DCM (3×40 ml). The resulting mixture was concentrated under reduced pressure. The residue was purified by silica gel column chromatography, eluted with PE/EtOAc (10:1) to afford 598-3 (3 g, 73.58%) as a yellow oil.

### 4. Synthesis of 598-4

Into a 50 mL round-bottom flask were added 598-3 (3.0 g, 9.551 mmol, 1 equiv), DMF (15 mL), 598-1 (1.91 g, 19.102 mmol, 2 equiv) and K₂CO₃ (3.96 g, 28.653 mmol, 3 equiv) at room temperature. The resulting mixture was stirred for overnight at 80° C. The reaction was quenched by the addition of H₂O (60 mL) at room temperature. The aqueous layer was extracted with EA (3×40 mL). The resulting mixture was concentrated under reduced pressure. The residue was purified by silica gel column chromatography, eluted with DCM/MeOH (50:1) to afford 598-4 (1.3 g, 40.84%) as a yellow oil.

### 5. Synthesis of 598-5

Into a 50 mL round-bottom flask were added 598-4 (1.3 g, 3.900 mmol, 1 equiv), THF (1 mL), 1M HC (aq. 12 mL) at room temperature. The resulting mixture was stirred for 1 h at 60° C. The mixture was neutralized to pH=7 with sat. NH₄HCO3 (12 mL). The aqueous layer was extracted with EA (3×30 mL). The resulting mixture was concentrated under reduced pressure. The residue was purified by Prep-TLC (DCM/MeOH=20:1) to afford 598-5 (250 mg, 21.3%) as a yellow oil.

### 6. Synthesis of 598-6

Into a 50 mL round-bottom flask were added 598-5 (250 mg, 0.871 mmol, 1 equiv), DCE (5 mL), 244b (210.80 mg, 0.871 mmol, 1 equiv), STAB (369.30 mg, 1.742 mmol, 2 equiv) and

AcOH (52.26 mg, 0.871 mmol, 1 equiv) at room temperature. The resulting mixture was stirred for 2 h at room temperature. The reaction was quenched by the addition of sat. NH₄Cl (5 mL) at room temperature. The aqueous layer was extracted with DCM/MeOH=10:1 (3×10 mL). The resulting mixture was concentrated under reduced pressure. The residue was purified by Prep-TLC (DCM/MeOH=15:1) to afford 598-6 (150 mg, 34.30%) as a yellow oil.

### 7. Synthesis of 598-7

Into a 100 mL round-bottom flask added 598-6 (150 mg, 0.292 mmol, 1 equiv), DCM (5 mL), pyridine (138.62 mg, 1.752 mmol, 6 equiv) and triphosgene (34.67 mg, 0.117 mmol, 0.4 equiv) at 0° C. The resulting mixture was stirred for 5 min at 0° C. The reaction was quenched with sat. NaHCO₃ (5 mL) at room temperature. The aqueous layer was extracted with DCM/MeOH=10:1 (3×5 mL). The resulting mixture was concentrated under reduced pressure. The residue was purified by Prep-TLC (DCM/MeOH=15:1) to afford 598-7 (50 Ig, 31.67%) as a yellow oil.

### 8. Synthesis of 598

The 598-7 (50 mg) was purified by Chiral separation with the following conditions (Column: CHIRALPAK IH, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 15 min; Wave Length: 220/254 am; RT1 (min): 4.66; RT2 (min): 11.99; The first peak was the product. Sample Solvent: EtOH:DCM=1:1-HPLC; Injection Volume: 2.75 mL; Number of Runs: 1) to afford 598 (9.4 mg, 18.44%) as a yellow solid.

LC-MS-598: (ES, m/z): [M+H]⁺ 540

H-NMR-598: 1H NMR (300 MHz, CD3OD-d4 ppm) δ 1.20-1.25 (m, 3H), 2.01-2.07 (m, 1H), 2.24-2.30 (m, 4H), 2.98-3.03 (m, 1H), 3.15 (s, 1H), 3.33 (s, 1H), 3.43-3.47 (m, 1H), 3.49-3.52 (m, 1H), 3.56-3.59 (m, 3H), 3.63-3.66 (m, 1H), 3.83-3.86 (m, 1H), 4.28-4.31 (m, 1H), 7.10-7.13 (m, 2H), 7.27-7.29 (m, 1H), 7.48-7.52 (m, 1H), 7.64-7.66 (d, 2H), 7.74 (s, 1H), 8.37 (s, 1H).

### Example 592. Synthesis of Compound 599

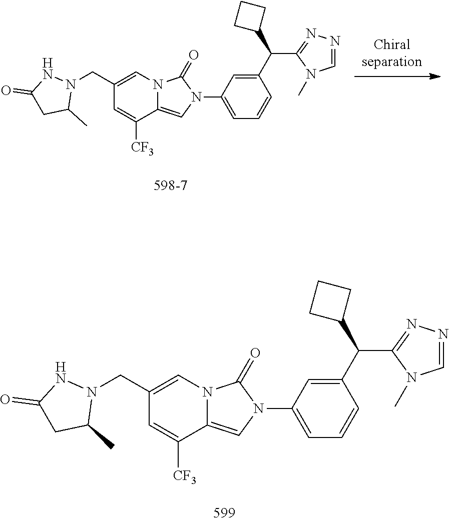 [SMILES: CC1CC(=O)NN1Cc1cc(C(F)(F)F)c2cn(-c3cccc([C@H](c4nncn4C)C4CCC4)c3)c(=O)n2c1 ; C[C@H]1CC(=O)NN1Cc1cc(C(F)(F)F)c2cn(-c3cccc([C@H](c4nncn4C)C4CCC4)c3)c(=O)n2c1]

### 1. Synthesis of 599

The 598-7 (50 mg) was purified by Chiral separation with the following conditions (Column: CHIRALPAK IH, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 15 min; Wave Length: 220/254 nm; RT1 (min): 4.66; RT2 (min): 11.99; The second peak was the product. Sample Solvent: EtOH:DCM=1:1-HPLC; Injection Volume: 2.75 mL; Number of Runs: 1) to afford 598 (11.0 mg, 22.00%) as a yellow solid.

LC-MS: (ES, m/z): [M+H]⁺ 540

H-NMR: 1H NMR (300 MHz, CD3OD-d4 ppm) δ 1.26-1.31 (m, 3H), 1.75-1.82 (m, 1H), 1.88-2.03 (m, 4H), 2.06-2.07 (m, 1H), 2.25-2.30 (m, 1H), 2.98-3.15 (m, 1H), 3.28-3.33 (m, 1H), 3.45-3.50 (m, 1H), 3.52-3.56 (m, 3H), 3.62-3.66 (m, 1H), 3.83-3.86 (m, 1H), 4.28-4.31 (m, 1H), 7.09-7.12 (m, 2H), 7.27-7.29 (m, 1H), 7.48-7.52 (m, 1H), 7.64-7.66 (m, 2H), 7.74 (s, 1H), 8.37 (s, 1H).

### Example 593. Synthesis of Compound 600

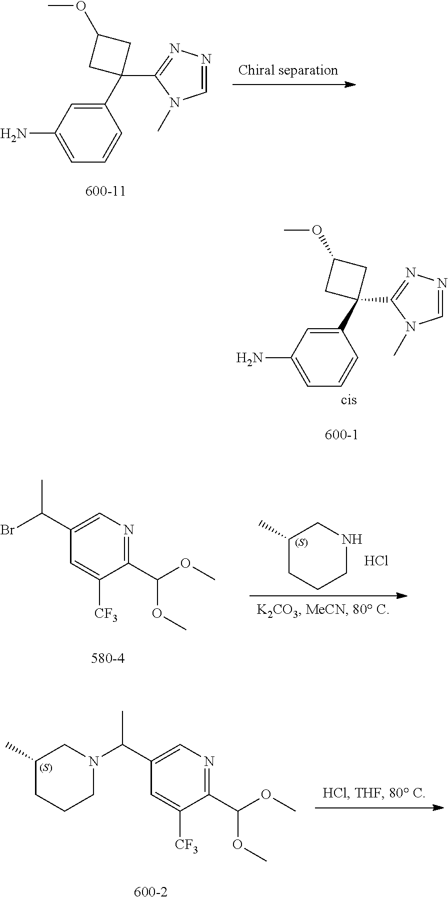

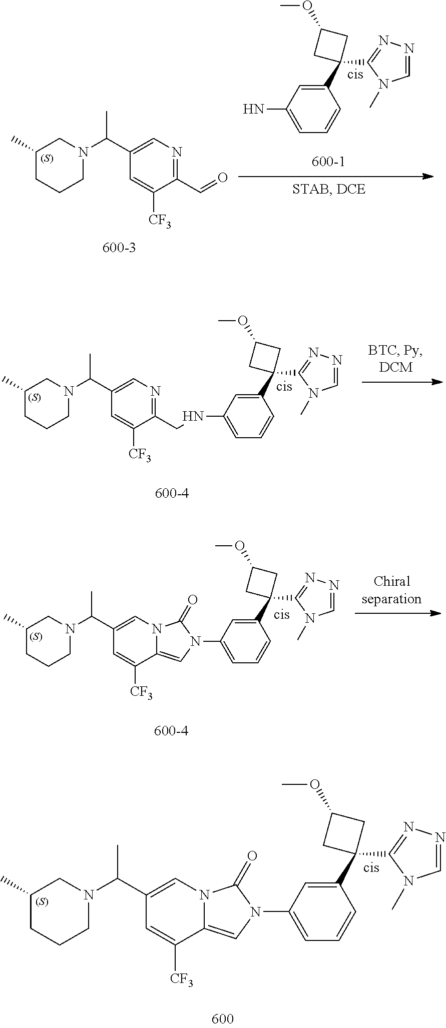

### 1. Synthesis of 600-1

The 600-11 (10 g) was purified by Prep-SFC with the following conditions (Column: Lux 5 um Cellulose-3, 3*25 cm, 5 μm; Mobile Phase A: CO2, Mobile Phase B: MEOH (0.1% 21M NH₃-MEOH); Flow rate: 70 mL/min; Gradient: isocratic 25% 13; Column Temperature (° C.): 35; Back Pressure (bar): 100; Wave Length: 220 nm; RT1 (min): 4.5; RT2 (min): 6.8; Sample Solvent: MeOH-Preparative; Injection Volume: 1.9 mL; Number Of Runs: 16, the first peak is product) to 600-1 (3.3 g, 33%) as a white solid.

### 2. Synthesis of 600-2

To a stirred solution of 580-4 (1.5 g, 4.571 mmol, 1 equiv) and (S)-3-methylpiperidine hydrochloride (1.24 g, 9,142 mmol, 2 equiv) in MeCN (20 mL) was added K₂CO₃ (1.90 g, 13,713 mmol, 3 equiv) at room temperature. The resulting mixture was stirred for 5 h at 80° C. under nitrogen atmosphere. The reaction was quenched with water (80 mL) at room temperature. The aqueous layer was extracted with EtOAc (3×50 mL). The resulting mixture was concentrated under reduced pressure. The residue was purified by Prep-TLC (CH2Cl₂/MeOH 30:1) to afford 600-2 (950 mg, 53.99%) as a light yellow oil.

### 3. Synthesis of 600-3

A solution of 600-2 (930 mg, 2.685 mmol, 1 equiv) and HCl (10 mL, 1M) in THF (10 mL) was stirred for 5 h at 80° C. The mixture was neutralized to pH 7 with saturated NaHCO₃ (aq.). The aqueous layer was extracted with EtOAc (3×50 mL). The resulting mixture was concenrated under reduced pressure. The residue was purified by Prep-TLC (CH₂Cl₂/MeOH 25:1) to afford 600-3 (730 mg, 81.48%) as a yellow oil.

### 4. Synthesis of 600-4

A solution of 600-3 (360 mg, 1.199 mmol, 1 equiv) and 600-1 (309.65 mg, 1.199 mmol, 1 equiv) in DCE (8 mL) was stirred overnight at room temperature. To the above mixture was added STAB (508.11 mg, 2,398 mmol, 2 equiv) at room temperature. The resulting mixture was stirred for additional 5 h at room temperature. The mixture was quenched with saturated NaHCO₃ (aq.) (50 mL). The aqueous layer was extracted with CH₂Cl₂ (3×30 mL). The resulting mixture was concentrated under reduced pressure. The residue was purified by Prep-TLC (CH₂Cl₂/MeOH 20:1) to afford 600-14 (430 mg, 59.49%) as a yellow solid.

### 5. Synthesis of 600-5

To a stirred solution of 600-4 (430 mg, 0,792 mmol, 1 equiv) and Pyridine (376.08 mg, 4,752 mmol, 6 equiv) in DCM (10 mL) was added Triphosgene (87.00 mg, 0.293 mmol, 0.37 equiv) at room temperature. The resulting mixture was stirred for 10 min at room temperature. The mixture was quenched with saturated NaHCO₃ (aq.) (80 mL) The aqueous layer was extracted with DCM (3×30 mL). The resulting mixture was concentrated under reduced pressure. The residue was purified by Prep-TLC (CH₂Cl₂/MeOH 20:1) to afford 600-15 (310 mg, 65.36%) as a yellow solid.

### 6. Synthesis of 600-0

The 600-5 (310 mg) was purified by Prep-Chiral-HPLC with the following conditions (Column: (R, R)-WHELK-O1-Kromasi, 5*25 cm, 5 μm; Mobile Phase A: Hex (0.5% 2M NH₃-MeOH), Mobile Phase B: EtOH:DCM=1:1; F ow rate: 20 mL/min; Gradient: 30% B to 30% B in 28 min; Wave Length: 220/254 nm; RT1 (min): 18.04; RT2 (min): 22.83; the first peak is product) to afford 600 (74.9 mg, 25.14%) as a yellow solid,

LC-MS: (ES, m/z): [M+H]⁺ 569. H-NMR: (400 MHz, DMSO-d6, δ ppm): 0.72-0.98 (m, 4H), 1.20-1.35 (m, 3H), 1.35-1.48 (m, 1H), 1.48-1.72 (m, 4H), 1.98-2.13 (m, 1H), 2.74-2.83 (m, 4H), 3.08-3.13 (m, 2H), 3.16 (s, 3H), 3.24 (s, 3H), 3.41-3.44 (m, 1H), 4.08-4.11 (m, 1H), 7.07 (s, 1H), 7.24-7.26 (d, 1H), 7.39 (s, 1H), 7.48-7.52 (m, 1H), 7.59 (s, 1H), 7.65-7.67 (m, 1H), 7.78 (s, 1H), 8.31 (s, 1H).

### Example 594. Synthesis of Compound 601

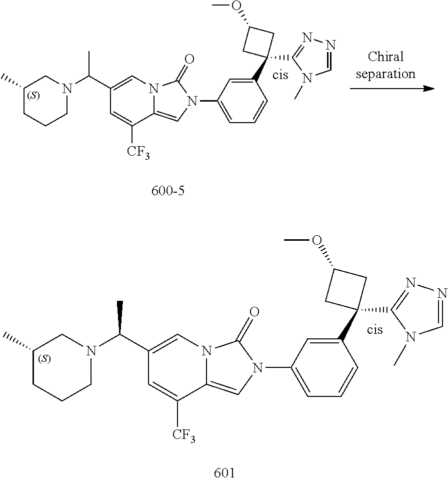

### 1. Synthesis of 601

The 600-5 (310 mg) was purified by Prep-Chiral-HPLC with the following conditions (Column: (R, R)-WHELK-O1-Kromasi, 5*25 cm, 5 μm; Mobile Phase A: Hex (0.5% 2M NH3-MeOH), Mobile Phase B: EtOH:DCM=1:1; Flow rate: 20 mL/min; Gradient: 30% B to 30% B in 28 min; Wave Length: 220/254 nm; RT1 (min): 18.04; RT2 (min): 22.83, the second peak is product) to afford 601 (92.5 mg, 30.02%) as a yellow solid.

LC-MS-601: (ES, m/z): [M+H]⁺ 569

H-NMR-601: (400 MHz, DMSO-d6, δ ppm): 0.72-0.98 (m, 4H), 1.20-1.35 (m, 3H), 1.35-1.48 (m, 1H), 1.48-1.72 (m, 4H), 1.93-2.03 (m, 1H), 2.74-2.83 (m, 4H), 3.08-3.13 (m, 2H), 3.16 (s, 3H), 3.24 (s, 3H), 3.41-3.46 (m, 1H), 4.06-4.11 (m, 1H), 7.08 (s, 1H), 7.24-7.26 (d, 1H), 7.38 (s, 1H), 7.48-7.52 (m, 1H), 7.58 (s, 1H), 7.65-7.67 (m, 1H), 7.79 (s, 1H), 8.31 (s, 1H).

### Example 595. Synthesis of Compound 602

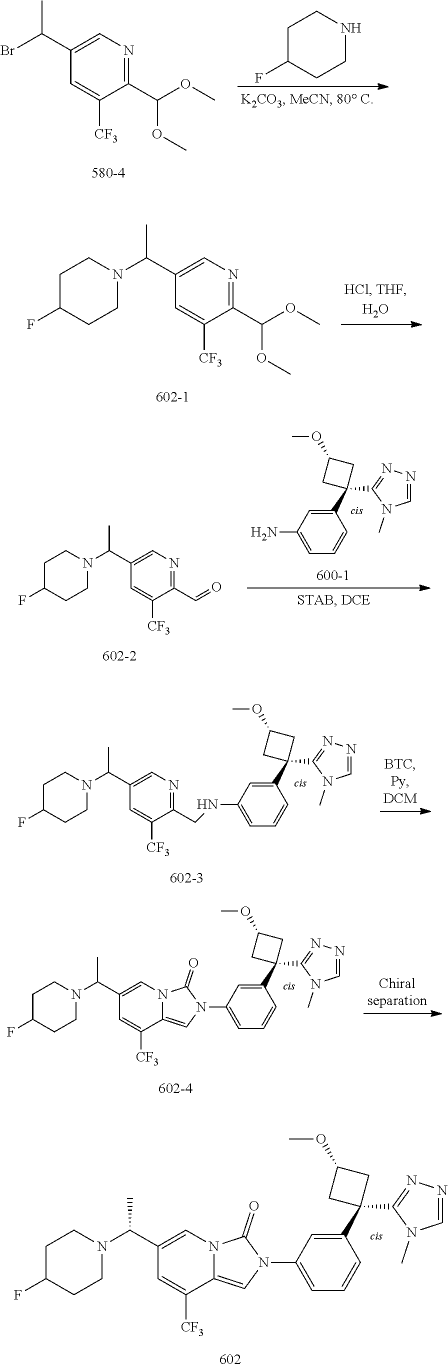

### 1. Synthesis of 602-1

To a stirred mixture of 580-4 (1.5 g, 4,571 mmol, 1 equiv) and 4-fluoropiperidine (0.57 g, 5,485 mmol, 1.2 equiv) in MeCN (20 mL) was added K2CO₃ (1.26 g, 9.142 mmol, 2 equiv) at room temperature. The resulting mixture was stirred overnight at 80° C. The mixture was allowed to cool down to room temperature. The reaction was quenched with water (100 mL) at room temperature. The aqueous layer was extracted with CH₂Cl₂ (3×50 mL). The resulting mixture was concentrated under vacuum. The residue was purified by Prep-TLC (Cl₂Cl₂/MeOH 35:1) to afford 602-1 (1 g, 62.44%) as a yellow oil,

### 2. Synthesis of 602-2

To a stirred mixture of 602-1 (1 g, 2,854 mmol, 1 equiv) in THF (5 mL) was added 1M HCl (10 mL) at room temperature. The resulting mixture was stirred overnight at 80° C. The mixture was neutralized to pH 7 with saturated NaHCO₃ (aq.). The aqueous layer was extracted with EtOAc (3×50 mL). The residue was purified by Prep-TLC (CH₂Cl₂/MeOH 30:1) to afford 602-2 (820 mg, 94.41%) as a yellow oil.

### 3. Synthesis of 602-3

A solution of 602-2 (450 mg, 1.479 mmol, 1.2 equiv) and 600-1 (318.35 mg, 1.233 mmol, 1 equiv) in DCE (4 mL) was stirred overnight at room temperature. To the above mixture was added STAB (522.38 mg, 2,465 mmol, 2 equiv) at room temperature. The resulting mixture was stirred for additional 4 h at room temperature. The reaction was quenched with saturated NaHCO₃ (aq.) (80 mL) at room temperature. The aqueous layer was extracted with CH2Cl20 (3×40 mL). The resulting mixture was concentrated under vacuum. The residue was purified by Prep-TLC (CH₂Cl₂/MeOH 15:1) to afford 602-3 (310 mg, 46.02%) as a white solid.

### 4. Synthesis of 602-4

To a stirred mixture of 602-3 (310 mg, 0.567 mmol, 1 equiv) and Pyridine (269.16 mg, 3.402 mmol, 6 equiv) in DCM (3 mL) was added Triphosgene (63.95 mg, 0.215 mmol, 0.38 equiv) at room temperature. The resulting mixture was stirred for 10 min at room temperature. The reaction was quenched with saturated NaHCO₃ (aq.) (80 mL) at room temperature. The aqueous layer was extracted with CH₂Cl₂ (3×40 mL). The resulting mixture was concentrated under reduced pressure. The residue was purified by Prep-TLC (CH₂Cl₂/MeOH 15:1) to afford 602-4 (200 mg, 61.59%) as a yellow solid.

### 5. Synthesis of 602

The 602-4 (200 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 55% B to 55% B in 14 min; Wave Length: 220/254 nm; RT1 (min): 9.69: RT2 (min): 11.69; the first peak is product) to afford 602 (34.7 mg, 17.35%) as a yellow solid

LC-MS: (ES, m/z): [M+H]⁺ 558. H-NMR: (400 MHz, DMSO-d₆, 3 ppm): 1.22-1.27 (m, 3H), 1.64-1.78 (m, 2H), 1.78-1.93 (m, 2H), 2.34-2.36 (m, 2H), 2.50-2.68 (m, 2H), 2.78-2.83 (m, 2H), 3.08-3.15 (m, 2H), 3.17 (s, 3H), 3.24 (s, 3H), 3.49-3.51 (m, 1H), 4.04-4.12 (m, 1H), 4.59-4.72 (m, 1H), 7.07 (s, 1H), 7.24-7.26 (d, 1H), 7.39 (s, 1H), 7.48-7.52 (m, 1H), 7.61 (s, 1H), 7.65-7.67 (d, 1H), 7.78 (s, 1H), 8.34 (s, 1H).

### Example 596. Synthesis of Compound 603

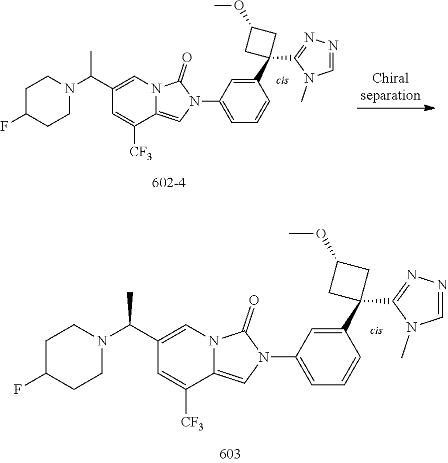 [SMILES: CO[C@H]1C[C@@](c2cccc(-n3cc4c(C(F)(F)F)cc(C(C)N5CCC(F)CC5)cn4c3=O)c2)(c2nncn2C)C1 ; CO[C@H]1C[C@@](c2cccc(-n3cc4c(C(F)(F)F)cc([C@H](C)N5CCC(F)CC5)cn4c3=O)c2)(c2nncn2C)C1]

### 1. Synthesis of 603

The 602-4 (200 mg) was purified by Prep-Chiral-HPLC with the following conditions (Column: CHIRALPAK IC, 2*25 cm, 5 n; Mobile Phase A: Hex (0.5% 2M NH₃-MeOH), Mobile Phase B: EtOH:DCM=1:1; Flow rate: 20 mL/min; Gradient: 55% B to 55% B in 14 min; Wave Length: 220/254 nm; RT1 (min): 9.69; RT2 (min): 11.69; the second peak is product) to afford 602 (30.1 mg, 15.05%) as a yellow solid.

LC-MS-603: (ES, m/z): [M+H]⁺ 573

H-NMR-603: (400 MHz, DMSO-d₆, δ ppm): 1.22-1.27 (m, 3H), 1.64-1.78 (m, 2H), 1.78-1.93 (m, 2H), 2.34-2.36 (m, 2H), 2.50-2.68 (m, 2H), 2.78-2.83 (m, 2H), 3.08-3.15 (m, 2H), 3.17 (s, 3H), 3.24 (s, 3H), 3.49-3.51 (m, 1H), 4.04-4.12 (m, 1H), 4.59-4.72 (m, 1H), 7.07 (s, 1H), 7.24-7.26 (d, 1H), 7.39 (s, 1H), 7.48-7.52 (m, 1H), 7.61 (s, 1H), 7.65-7.67 (d, 1H), 7.78 (s, 1H), 8.31 (s, 1H).

### Example 597. Synthesis of Compound 604

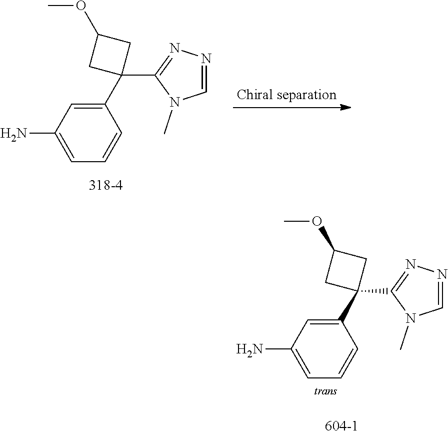 [SMILES: COC1CC(c2cccc(N)c2)(c2nncn2C)C1 ; CO[C@H]1C[C@](c2cccc(N)c2)(c2nncn2C)C1]

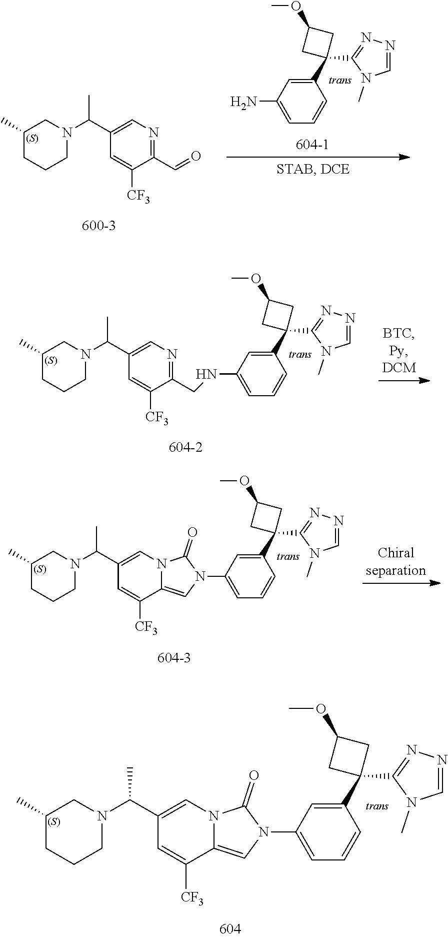

### 1. Synthesis of 604-1

The 318-4 (10 g) was purified by Prep-SFC with the following conditions (Column: Lux 5 um Cellulose-3, 3*25 cm, 5 μm; Mobile Phase A: CO2, Mobile Phase B: MEOH (0.1% 2M NH₃-MEOH); Flow rate: 70 mL/min; Gradient: isocratic 25% B; Column Temperature (° C.): 35; Back Pressure (bar): 100; Wave Length: 220 nm; RT1 (min): 4.5; RT2 (min): 6.8; Sample Solvent: MeOH-Preparative; Injection Volume: 1.9 mL; Number Of Runs: 16, the second peak is product) to 604-1 (4.8 g, 48%) as a white solid.

### 2. Synthesis of 604-2

A solution of 600-3 (360 mg, 1.199 mmol, 1 equiv) and 604-1 (309.65 mg, 1.199 mmol, 1 equiv) in DCE (3.6 mL) was stirred overnight at room temperature. To the above mixture was added STAB (508.11 mg, 2.398 mmol, 2 equiv) at room temperature. The resulting mixture was stirred for additional 5 h at room temperature. The mixture was quenched with saturated NaHCO₃ (aq.) (50 mL). The aqueous layer was extracted with CH₂Cl₂ (3×30 mL). The resulting mixture was concentrated under reduced pressure. The residue was purified by Prep-TLC (CH₂Cl₂/MeOH 20:1) to afford 604-2 (460 mg, 63.65%) as a yellow solid.

### 3. Synthesis of 604-3

To a stirred solution of 604-2 (460 mg, 0.848 mmol, 1 equiv) and Pyridine (402.31 mg, 5.088 mmol, 6 equiv) in DCM (4.3 mL) was added Triphosgene (93.07 mg, 0.314 mmol, 0.37 equiv) at room temperature. The resulting mixture was stirred for 10 min at room temperature. The mixture was quenched with saturated NaHCO₃ (aq.) (80 mL). The aqueous layer was extracted with CH₂Cl₂ (3×50 mL). The resulting mixture was concentrated under reduced pressure. The residue was purified by Prep-TLC (CH₂C/MeOH 20:1) to afford 604-3 (310 mg, 57.88%) as a yellow solid.

### 4. Synthesis of 604

The 604-3 (370 mg) was purified by Prep-Chiral-HPLC with the following conditions (Column: (R, R)-WHELK-O1-Kromasi, 5*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 30% B to 30% B in 23 min; Wave Length: 220/254 um; RT1 (min): 14.85; RT2 (min): 18.49, the first peak is product) to afford 604 (120.5 mg, 32.73%) as a yellow solid.

LC-MS-604: (ES, m/z): [M+H]⁺ 569

H-NMR-604: (400 MHz, DMSO-d6, δ ppm): 0.75-0.92 (m, 4H), 1.21-1.27 (m, 3H), 1.44-1.84 (m, 5H), 1.99-2.04 (m, 1H), 2.50-2.55 (m, 2H), 2.69-2.76 (m, 2H), 3.17 (s, 3H), 3.25 (s, 3H), 3.31-3.33 (m, 1H), 3.33-3.35 (m, 1H), 3.42-3.44 (m, 1H), 3.83-3.89 (m, 1H), 7.07-7.10 (m, 2H), 7.39 (s, 1H), 7.46-7.50 (m, 1H), 7.57 (s, 1H), 7.65-7.67 (m, 1H), 7.71 (s, 1H), 8.39 (s, 1H).

### Example 598. Synthesis of Compound 605

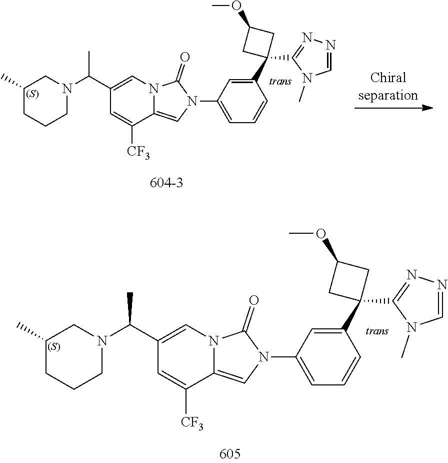

### 1. Synthesis of 605

The 604-3 (370 mg) was purified by Prep-Chiral-HPLC with the following conditions (Column: (R, R)-WHELK-O1-Kromasi, 5*25 cm, 5 μm; Mobile Phase A: Hex (0.5% 2M NH₃-MeOH), Mobile Phase B: EtOH:DCM=1:1; Flow rate: 20 mL/min; Gradient: 30% B to 30% B in 23 min; Wave Length: 220/254 nm; RT1 (min): 14.85; RT2 (min): 18.49, the second peak is product) to afford 605 (117.9 mg, 32.39%) as a yellow solid.

LC-MS-605: (ES, m/z): [M+H]⁺ 569

H-NMR-605: (400 MHz, DMSO-d6, δ ppm): 0.75-0.92 (m, 4H), 1.21-1.27 (m, 3H), 1.34-1.44 (m, 1H), 1.51-1.78 (m, 4H), 1.81-1.99 (m, 1H), 2.50-2.55 (m, 2H), 2.69-2.76 (m, 2H), 3.17 (s, 3H), 3.25 (s, 3H), 3.31-3.33 (m, 1H), 3.33-3.35 (m, 1H), 3.42-3.44 (m, 1H), 3.83-3.89 (m, 1H), 7.07-7.09 (m, 2H), 7.39 (s, 1H), 7.46-7.50 (m, 1H), 7.56 (s, 1H), 7.65-7.67 (m, 1H), 7.70 (s, 1H), 8.39 (s, 1H).

### Example 599. Synthesis of Compound 606

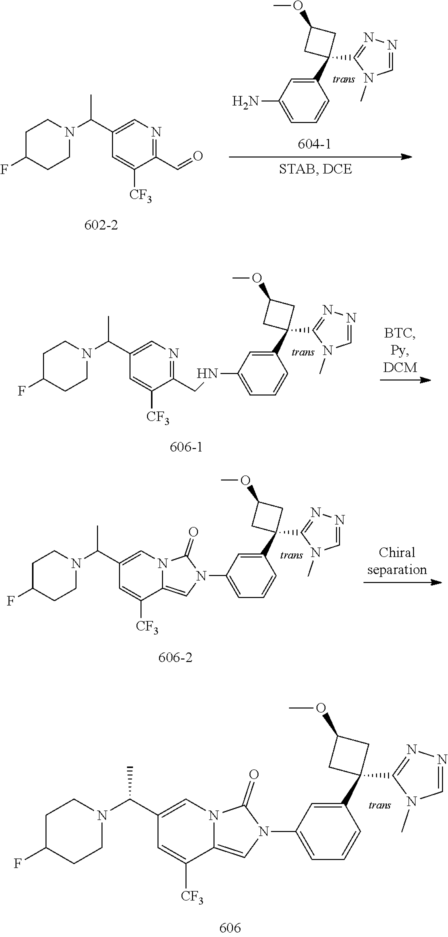 [SMILES: CC(c1cnc(C=O)c(C(F)(F)F)c1)N1CCC(F)CC1 ; CO[C@H]1C[C@](c2cccc(N)c2)(c2nncn2C)C1 ; CO[C@H]1C[C@](c2cccc(NCc3ncc(C(C)N4CCC(F)CC4)cc3C(F)(F)F)c2)(c2nncn2C)C1 ; CO[C@H]1C[C@](c2cccc(-n3cc4c(C(F)(F)F)cc(C(C)N5CCC(F)CC5)cn4c3=O)c2)(c2nncn2C)C1 ; CO[C@H]1C[C@](c2cccc(-n3cc4c(C(F)(F)F)cc([C@@H](C)N5CCC(F)CC5)cn4c3=O)c2)(c2nncn2C)C1]

### 1. Synthesis of 606-1

A solution of 602-2 (400 mg, 1,316 mmol, 1.067 equiv) and 604-1 (318.35 mg, 1.233 mmol, 1 equiv) in DCE (5 mL) was stirred overnight at room temperature. To the above mixture was added STAB (522.38 mg, 2.465 mmol, 2 equiv) at room temperature. The resulting mixture was stirred for additional 4 h at room temperature. The reaction was quenched with saturated NaHCO3 (aq.) (80 mL) at room temperature. The aqueous layer was extracted with CH₂Cl₂ (3×50 mL). The resulting mixture was concentrated under vacuum. The residue was purified by Prep-TLC (CH₂Cl₂/MeOH 15:1) to afford 606-1 (500 mg, 74.22%) as a white solid.

### 2. Synthesis of 606-2

To a stirred mixture of 606-1 (480 mg, 0.878 mmol, 1 equiv) and Pyridine (416.76 mg, 5.268 mmol, 6 equiv) in DCM (5 mL) was added Triphosgene (99.02 mg, 0.334 mmol, 0.38 equiv) at room temperature. The resulting mixture was stirred for 10 min at room temperature. The reaction was quenched with saturated NaHCO₃ (aq.) (80 mL) at room temperature. The aqueous layer was extracted with CH₂Cl₂ (3×40 mL). The resulting mixture was concentrated under vacuum. The residue was purified by Prep-TLC (CH₂Cl₂/MeOH 15:1) to afford 606-2 (330 mg, 65.63%) as a yellow solid.

### 3. Synthesis of 606

The 606-2 (330 mg) was purified by Prep-CHIRAL-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min Gradient: 55% B to 55% B in 15 min; Wave Length: 220/254 nm; RT1 (min): 7.49; RT2 (min): 10.74; the first peak is product) to afford 606 (109.5 mg, 33.18%) as a yellow solid.

LC-MS-606: (ES, m/z): [M+H]⁺ 558

H-NMR-606: (400 MHz, DMSO-d₆, 3 ppm): 1.22-1.27 (m, 3H), 1.63-1.78 (m, 2H), 1.78-1.93 (m, 2H), 2.34-2.36 (m, 2H), 2.50-2.57 (m, 4H), 3.16 (s, 3H), 3.24 (s, 3H), 3.31-3.35 (m, 2H), 3.49-3.51 (m, 1H), 3.51 (m, 1H), 3.84-3.88 (m, 1H), 4.59-4.72 (m, 1H), 7.07-7.09 (m, 2H), 7.39 (s, 1H), 7.46-7.50 (m, 1H), 7.64 (s, 1H), 7.66-7.69 (d, 1H), 7.70 (s, 1H), 8.39 (s, 1H).

### Example 600. Synthesis of Compound 607

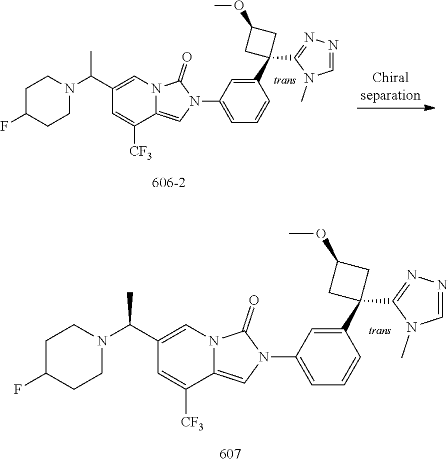 [SMILES: CO[C@H]1C[C@](c2cccc(-n3cc4c(C(F)(F)F)cc(C(C)N5CCC(F)CC5)cn4c3=O)c2)(c2nncn2C)C1 ; CO[C@H]1C[C@](c2cccc(-n3cc4c(C(F)(F)F)cc([C@H](C)N5CCC(F)CC5)cn4c3=O)c2)(c2nncn2C)C1]

### 1. Synthesis of 607

The 606-2 (330 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 55% B to 55% B in 15 min; Wave Length: 220/254 nm; RT1 (min): 7.49; RT2 (min): 10.74; the second peak is product) to afford 607 (143.5 mg, 43.48%) as a yellow solid.

LC-MS-607: (ES, m/z): [M+H]⁺ 573

H-NMR-607: (400 MHz, DMSO-d₆, δ ppm): 1.22-1.27 (m, 3H), 1.63-1.78 (m, 2H), 1.78-1.93 (m, 2H), 2.34-2.36 (m, 2H), 2.50-2.57 (m, 4H), 3.16 (s, 3H), 3.24 (s, 3H), 3.31-3.35 (m, 2H), 3.49-3.51 (m, 1H), 3.51 (m, 1H), 3.84-3.88 (m, 1H), 4.59-4.72 (m, 1H), 7.07-7.09 (m, 2H), 7.39 (s, 1H), 7.46-7.50 (m, 1H), 7.64 (s, 1H), 7.66-7.69 (d, 1H), 7.70 (s, 1H), 8.39 (s, 1H).

### Example 601. Synthesis of Compound 608

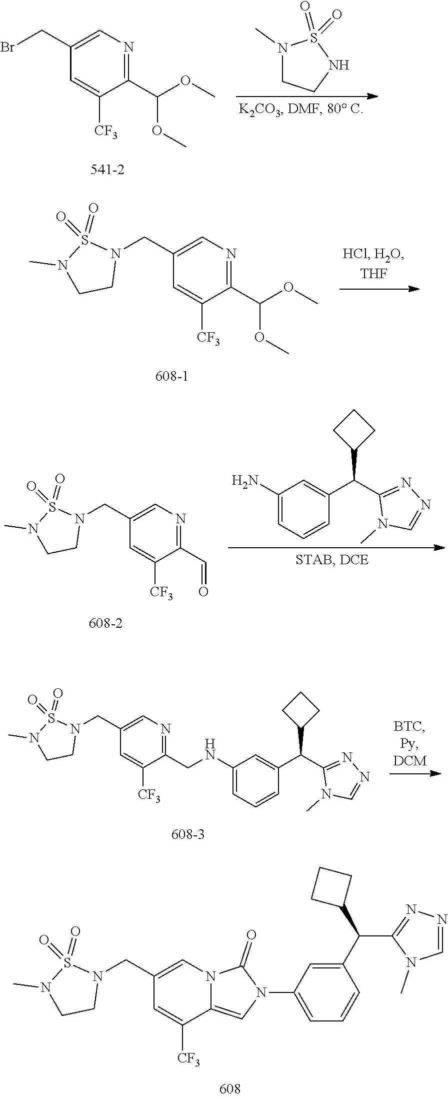

### 1. Synthesis of 608-1

Into a 40 mL vial were added 541-2 (1.2 g, 3.820 mmol, 1 equiv), 2-methyl-1lambda6,2,5-thiadiazolidine-1,1-dione (0.78 g, 5.730 mmol, 1.5 equiv), K₂CO₃ (1.06 g, 7,640 mmol, 2 equiv) and DMF (15 mL) at room temperature. The resulting mixture was stirred for overnight at 80° C. The resulting mixture was diluted with water (100 mL). The aqueous layer was extracted with EtOAc (3×40 mL). The resulting mixture was concentrated under vacuum. The residue was purified by Prep-TLC (DCM/MeOH 50:1) to afford 608-1 (700 mg, 46.63%) as a colorless oil.

### 2. Synthesis of 608-2

Into a 40 mL vial were added 608-1 (700 mg, 1.895 mmol, 1 equiv) 1M HCl (7 mL) and H₂O (2 mL) at room temperature. The resulting mixture was stirred for 2 h at 80° C. The residue was basified to pH 8 with sat. NH₄HCO₃ (aq.) (100 mL). The aqueous layer was extracted with EtOAc (3×30 mL). The resulting mixture was concentrated under vacuum. The residue was purified by Prep-TLC (DCM/MeOH 60:1) to afford 608-2 (400 mg, 61.37%) as a colorless oil.

### 3. Synthesis of 608-3

Into a 50 mL round-bottom flask were added 608-2 (400 mg, 1.237 mmol, 1 equiv), I-2 (359.79 rug, 1.484 mmol, 1.2 equiv), STAB (524.46 mg, 2.474 mmol, 2 equiv) and DCE (10 mL) at room temperature. The resulting mixture was stirred for overnight at room temperature. The reaction was quenched with sat. NH₄Cl (aq.) (50 mL) at room temperature. The aqueous layer was extracted with EtOAc (3×20 mL). The resulting mixture was concentrated under vacuum. The residue was purified by Prep-TLC (DCM/MeOH=20:1) to afford 608-3 (400 mg, 55.29%) as a colorless oil.

### 4. Synthesis of 608

Into a 50 ml round-bottom flask were added 608-3 (380 mg, 0.691 mmol, 1 equiv), DCM (10 mL, 157.306 mmol, 227.52 equiv) and pyridine (437.51 mg, 5.528 mmol, 8 equiv) at room temperature. To the above mixture was added triphosgene (102.58 mg, 0.345 mmol, 0.5 equiv) at room temperature. The resulting mixture was stirred for 3 min at room temperature. The reaction was quenched with sat. NaHCO₃ (aq.) (50 mL) at room temperature. The aqueous layer was extracted with EtOAc (3×20 mL). The resulting mixture was concentrated under vacuum. The crude product (300 mg) was purified by Prep-HPLC with the following conditions (Column: X Bridge Prep OBD C1 8 Column, 30*150 mm, 5 μm; Mobile Phase A: water (10 mmol/L NH4HCO₃), Mobile Phase B: ACN; Flow rate: 60 mL/min; Gradient: 30% B to 50% B in 8 min; Wave Length: 254 nm; RT1 (min): 7.58) to afford 608 (53.8 mg, 13.42%) as a yellow solid.

LC-MS-608: (ES, m/z): [M+H]⁺ 576

H-NMR-608: 1H NMR (400 MHz, DMSO-d6 ppm) δ 1.72-1.81 (m, 5H), δ 2.06-2.10 (m, 1H), δ 2.67 (s, 3H), δ 3.19-3.35 (m, 4H), δ 3.43 (s, 3H), δ 4.02 (s, 2H), δ 4.25-4.28 (d, 1H), δ 7.01 (s, 1H), δ 7.19-7.21 (d, 1H), δ 7.35 (s, 1H), δ 7.43-7.47 (t, 1H), δ 7.69-7.73 (m, 2H), δ 7.87 (s, 1H), 68.32 (s, 1H).

### Example 602. Synthesis of Compound 609

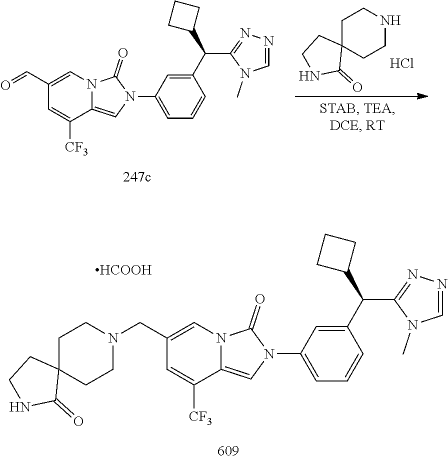

### 1. Synthesis of 609

To a stirred solution of 247c (2 g, 4,391 mmol, 1 equiv) and 2,8-diazaspiro[4,5]decan-1-one hydrochloride (2.51 g, 13.164 mmol, 3.00 equiv) in DCE (40 mL) were added TEA (1.56 g, 15.369 mmol, 3.5 equiv) at room temperature. The resulting mixture was stirred for 3 h at room temperature. To the above mixture was added STAB (1.86 g, 8.782 mmol, 2 equiv) at room temperature. The resulting mixture was stirred overnight at room temperature. The reaction was quenched with water (100 mL) at room temperature. The aqueous layer was extracted with DCM (2×50 mL). The resulting mixture was concentrated under vacuum. The residue was purified by reverse flash chromatography with the following conditions: column, C18 silica gel; mobile phase, MeCN in water (0.1% FA), 10% to 55% gradient in 20 min; detector, UV 254 nm. This resulted in 609 (242.5 mg, 8.96%) as a yellow solid.

LC-MS-609: (ES, m/z): [M+H]⁺ 594

H-NMR-609: (400 MHz, DMSO-d6, δ ppm): 1.32-1.35 (m, 2H), 1.64-1.88 (m, 7H), 1.90-1.97 (m, 2H), 2.05-2.11 (m, 3H), 2.78-2.81 (m, 2H), 3.13-3.27 (m, 3H), 3.31 (s, 2H), 3.43 (s, 3H), 4.25-4.27 (d, 1H), 7.03 (s, 1H), 7.18-7.20 (d, 1H), 7.32 (s, 1H), 7.42-7.46 (m, 1H), 7.55 (s, 1H), 7.68-7.70 (m, 2H), 7.73 (s, 1H), 8.16 (s, 1H), 8.33 (s, 1H).

### Example 603. Synthesis of Compound 610_P1

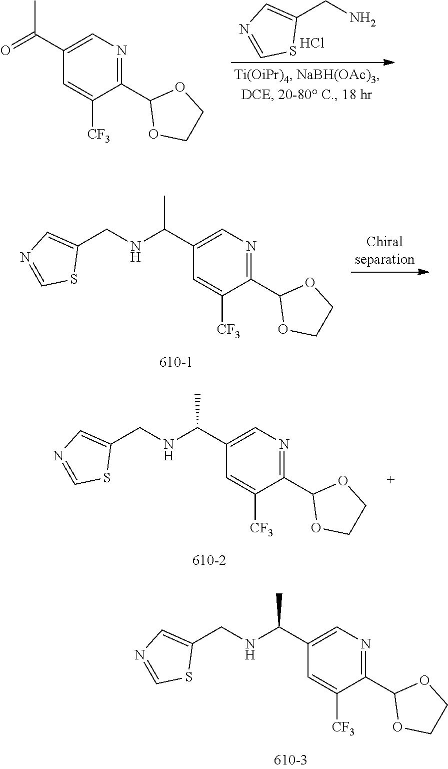 [SMILES: CC(=O)c1cnc(C2OCCO2)c(C(F)(F)F)c1 ; NCc1cncs1 ; Cl ; CC(NCc1cncs1)c1cnc(C2OCCO2)c(C(F)(F)F)c1 ; C[C@@H](NCc1cncs1)c1cnc(C2OCCO2)c(C(F)(F)F)c1 ; C[C@H](NCc1cncs1)c1cnc(C2OCCO2)c(C(F)(F)F)c1]

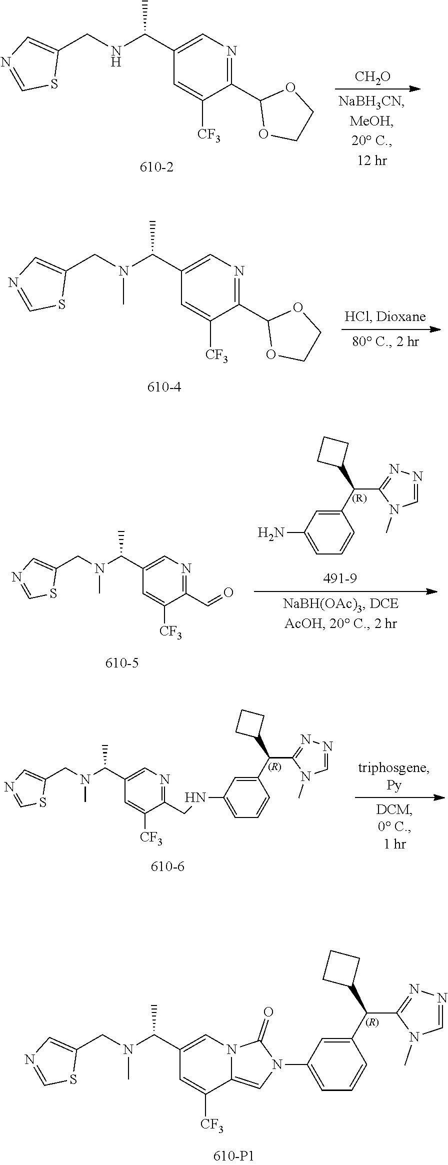

### 1. Synthesis of 610-1

To a solution of 1-(6-(1,3-dioxolan-2-yl)-5-(trifluoromethyl)pyridin-3-yl)ethan-1-one (1.3 g, 4.98 mmol, 1 equiv) and thiazol-5-ylmethanamine (1.12 g, 7.47 mmol, 15 equiv, HCl) in DC. (15 mL) was added tetraisopropoxytitanium (4.24 g, 14.93 mmol, 441 mL, 3 equiv). The mixture was heated to 80° C. and stirred at 80° C. for 12 hr. Then sodium; triacetoxyboranuide (4.22 g, 19.91 mmol, 4 equiv) was added to reaction mixture at 20° C. The mixture was stirred at 80° C. for 6 hr under N₂. The mixture was poured into NaHCO₃ aq, (20 mL), extracted with EtOAc (10 mL×3). The combined organic layer was washed with brine (30 mL), dried over anhydrous Na₂SO₄, filtered and concentrated under reduced pressure to give a crude. The residue was purified by column chromatography (SiO₂, Petroleum ether/Ethyl acetate=50/1 to 0/1) to afford 610-1 (940 mg, 52.56% yield) as a yellow oil.

¹H-NMR-610-1: (400 MHz, CHLOROFORM-d) δ 8.83 (d, J=2.0 Hz, 1H), 8.74 (s, 1H), 8.03 (d, J=2.0 Hz, 1H), 7.67 (s, 1H), 6.27 (d, J=1.2 Hz, 1H), 4.38-4.35 (m, 2H), 4.15-4.12 (m, 2H), 4.02-3.97 (m, 1H), 3.93-3.82 (m, 2H), 1.40 (d, J=6.8 Hz, 3H).

### 2. Synthesis of 610-2 and 610-3

The 610-1 (940 mg) was purified by Chiral separation with the following conditions (column: Phenomenex-Cellulose-2 (250 mm*30 mm, 10 um); mobile phase: [Neu-ETOH]; B %: 20%-20%, 5 min Flow rate: 3.4 mL/min; Wave Length: 220/254 nm; RT1 (min): 1.603; RT2 (min): 1,700) to afford 610-2 (410 mg, 35.96% yield) as a yellow oil and 610-3 (450 mg, 39.47% yield) as a yellow oil.

¹H-NMR-610-2: (400 MHz, METHANOL-d₄) δ 8.89 (s, 1H), 8.78 (d, J=1.6 Hz, 1H), 8.18 (d, J=2.0 Hz, 1H), 7.66 (s, 1H), 6.16 (d, J=1.2 Hz, 1H), 4.27-4.23 (m, 2H), 4.08-4.05 (m, 2H), 4.02-3.99 (m, 1H), 3.96-3.83 (m, 2H), 1.41 (d, J=6.4 Hz, 3H).

¹H-NMR-610-3: (400 MHz, METHANOL-d₄) δ 8.89 (s, 1H), 8.78 (d, J=1.6 Hz, 1H), 8.18 (d, J=1.6 Hz, 1H), 7.66 (s, 1H), 6.16 (d, J=1.2 Hz, 1H), 4.27-4.23 (m, 2H), 4.08-4.05 (m, 2H), 4.02-3.99 (m, 1H), 3.96-3.83 (m, 2H), 1.41 (d, J=6.4 Hz, 3H).

### 3. Synthesis of 610-4

To a solution of 610-2 (200 mg 556 umol, 1 equiv) in MeOH (4 mL) was added HCHO (68 mg, 835 umol, 62 uL, 37% purity, 1.5 equiv) at 20° C. stirred for 1 hr. Then NaBH₃CN (69.95 mg, 1.11 mmol, 2 equiv) was added and stirred at 20° C. for 11 hr. The same scale reaction was conducted in parallel for 2 batches in total and work up with a small test scale (20 mg) together. The mixture was concentrated under reduced pressure to give a residue. The residue was purified by column chromatography (SiO₂, Petroleum ether/Ethyl acetate=50/1 to 0/1) to afford 610-4 (350 mg, 82.15% yield) as a yellow oil.

¹H-NMR-610-4: (400 MHz, METHANOL-d₄) δ 8.94 (s, 1H), 8.85 (s, 1H), 8.20 (d, J=1.2 Hz, 1H), 7.72 (s, 1H), 6.17 (d, J=1.2 Hz, 1H), 4.27-4.24 (m, 2H), 4.11-4.05 (m, 2H), 3.98 (q, J=7.2 Hz, 1H), 3.88-3.76 (m, 2H), 2.23 (s, 3H), 1.48 (d, J=6.8 Hz, 3H).

### 4. Synthesis of 610-5

To a solution of 610-4 (0.3 g, 803 umol, 1 equiv) in dioxane (L 5 mL) was added HCl (4M, 1.5 mL). The mixture was heated to 80° C. and stirred at 80° C. for 2 hr. The mixture was poured into NaHCO₃ aq. (10 mL) adjust to pH==8-9, extracted with EtOAc (5 mL×3). The combined organic layer was washed with brine (10 mL), dried over anhydrous Na2SO4, concentrated in vacuum to give 610-5 (320 mg, crude) as a yellow oil.

### 5. Synthesis of 610-6

To a solution of 610-5 (270 mg, 820 umol, 1 equiv) in DCE (5 mL) was added 491-9 (199 mg, 820 umol, 1 equiv), acetic acid (49.23 mg, 820 umol, 47 uL, 1 equiv) at 20° C. stirred for 1 h. Then sodium; triacetoxyboranuide (348 mg, 1.64 mmol, 2 equiv) was added and stirred at 20° C. for 1 hr. The mixture was poured into water (10 ml), extracted with DCM (5 ml×3). The combined organic layer was washed with brine (10 mL), dried over anhydrous Na₂SO₄, concentrated in vacuum to give a residue. The residue was purified by column chromatography (SiO₂, Petroleum ether/Ethyl acetate=20/1 to 0/1) and (DCM/MeOH=50/1 to 1/1) to afford 610-6 (430 mg, crude) as a yellow oil.

### 6. Synthesis of 610_P1

To a solution of 610-6 (380 mg, 684 umol 1 equiv) in DCM (5 mL) was added Py (325 mg, 4.10 mmol, 331 uL, 6 equiv) and bis(trichloromethyl) carbonate (102 mg, 342 umol, 0.5 equiv). The mixture was stirred at 0° C. for 1 hr. The same scale reaction was conducted and work up with a small test scale (50 mg) together. The reaction mixture was diluted with NaHCO₃ aq. (15 mL), extracted with DCM (10 mL×3). The combined organic layer was washed with brine (10 mL), dried over anhydrous Na₂SO₄, concentrated in vacuum to give a residue. The residue was purified by prep-HPLC (column: Phenomenex Luna C18 200*40 mm*10 um; mobile phase: [water (FA)-ACN]; B %: 10%-45%, 8 min, Wave Length: 220 am; RT1 (min): 8.5 min) to afford 610_P1 (154.1 mg, 34.24% yield) as a yellow solid.

MS-610_P1: (ES, m/z): [M+H]⁺ 582.2.

¹H-NMR-610_P1: (400 MHz, DMSO-d₆) δ 9.01 (s, 1H), 8.32 (s, 1H), 7.75-7.72 (m, 2H), 7.69-7.67 (m, 1H), 7.59 (s, 1H), 7.44 (t, J=7.6 Hz, 1H), 7.32 (s, 1H), 7.19-7.14 (m, 2H), 4.25 (d, J=10.8 Hz, 1H), 3.83-3.71 (m, 2H), 3.67-3.62 (m, 1H), 3.42 (s, 3H), 3.24-3.18 (m, 1H), 2.14 (s, 3H), 2.11-2.05 (m, 1H), 1.85-1.67 (m, 5H), 1.30 (d, J=6.4 Hz, 3H).

### Example 604. Synthesis of Compound 610_P2

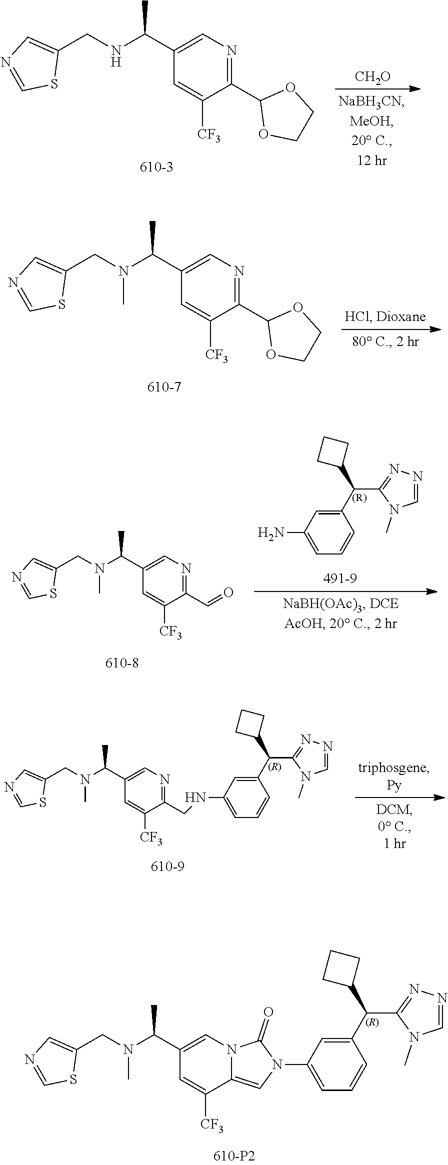

### 1. Synthesis of 610-7

To a solution of 610-3 (240 mg, 667.84 umol, 1 equiv) in MeOH (1 mL) was added HCHO (81.29 mg, 1.00 mmol, 37% purity, 1.5 equiv) at 20° C. stirred for 1 hr. Then NaBH₃CN (83.93 mg, 1.34 mmol, 2 equiv) was added and stirred at 20° C. for 11 hr. The same scale reaction was conducted with in parallel for 2 batches in total and work up with a small test scale (180 mg) together. The mixture was concentrated under reduced pressure to give a residue. The residue was purified by column chromatography (SiO₂, Petroleum ether/Ethyl acetate=50/1 to 0/1) to afford 610-7 (320 ag, 71.74% yield) as a yellow oil.

¹H-NMR-610-7: (400 MHz, METHANOL-d₄) δ 8.94 (s, 1H), 8.85 (s, 1H), 8.20 (d, J=1.2 Hz, 1H), 7.72 (s, 1H), 6.17 (d, J=1.2 Hz, 1H), 4.27-4.24 (m, 2H), 4.11-4.05 (m, 2H), 3.98 (q, J=7.2 Hz, 1H), 3.88-3.76 (m, 2H), 2.23 (s, 3H), 1.48 (d, J=6.8 Hz, 3H).

### 2. Synthesis of 610-8

To a solution of 610-7 (270 mg, 723.10 umol, 1 equiv) in dioxane (2 mL) was added HC (4M, 2 mL). The mixture was heated to 80° C. and stirred at 80° C. for 2 hr. The mixture was poured into NaHCO3 aq. (10 mL) to pH=8-9, extracted with EtOAc (5 mL×3). The combined organic layer was washed with brine (10 mL), dried over anhydrous Na₂SO₄, concentrated in vacuum to give 610-8 (320 mg, crude) as a yellow oil.

### 3. Synthesis of 610-9

To a solution of 610-8 (0.27 g, 820 umol, 1 equiv) in DCE (5 mL) was added 491-9 (199 mg, 820 umol, 1 equiv), acetic acid (49.23 mg, 8120 umol, 1 equiv) at 20° C. stirred for 1 hr. Then sodium; triacetoxyboranuide (347.51 mg, 1.64 mmol, 2 equiv) was added and stirred at 20° C. for 1 hr. The reaction was combined work up with a small test scale (50 mg) together. The mixture was poured into water (10 mL), extracted with DCM (5 ml×3). The combined organic layer was washed with brine (10 mL), dried over anhydrous Na₂SO₄, concentrated in vacuum to give a residue. The residue was purified by column chromatography (SiO₂, Petroleum ether/Ethyl acetate=20/1 to 0/1) and (DCM/MeOH=50/1 to 1/1) to afford 610-9 (490 mg, crude) as a yellow oil.

### 4. Synthesis of 610_P2

To a solution of 610-9 (440 mg, 791.85 umol, 1 equiv) in DCM (8 mL) was added Py (375.81 mg, 4.75 mmol, 383.48 uL, 6 equiv), bis(trichloromethyl) carbonate (117.49 mg, 395.93 umol, 0.5 equiv). The mixture was stirred at 0° C. for 1 hr. The reaction was combined with a small test scale (50 mg) together. The reaction mixture was diluted with NaHCO₃ aq. (15 mL), extracted with DCM (10 mL×3). The combined organic layer was washed with brine (10 mL), dried over anhydrous Na₂SO4, concentrated in vacuum to give a residue. The residue was purified by prep-HPLC (column: Phenomenex C18 75*30 mm*3 um; mobile phase: [water (NH₄HCO₃)-ACN]; B %: 45%-65%, 8 min, Wave Length: 220 nm; RT1 (min): 10 min) to afford 610_P2 (25.3 rug, 4.94% yield) as a yellow solid.

MS-610_P2: (ES, m/z): [M+H]⁺ 582.2.

¹H-NMR-610_P2: (400 MHz, DMSO-d₆) δ 9.01 (s, 1H), 8.32 (s, 1H), 7.75-7.73 (m, 2H), 7.69-7.67 (m, 1H), 7.59 (s, 1H), 7.44 (t, J=8.0 Hz, 1H), 7.30 (s, 1H), 7.19-7.18 (m, 1H), 7.14 (s, 1H), 4.25 (d, J=10.8 Hz, 1H), 3.83-3.71 (m, 2H), 3.67-3.63 (m, 1H), 3.42 (s, 3H), 3.24-3.18 (m, 1H), 2.14 (s, 3H), 2.11-2.05 (m, 1H), 1.80-1.67 (m, 5H), 1.30 (d, J=6.8 Hz, 3H).

### Example 605. Synthesis of Compound 611

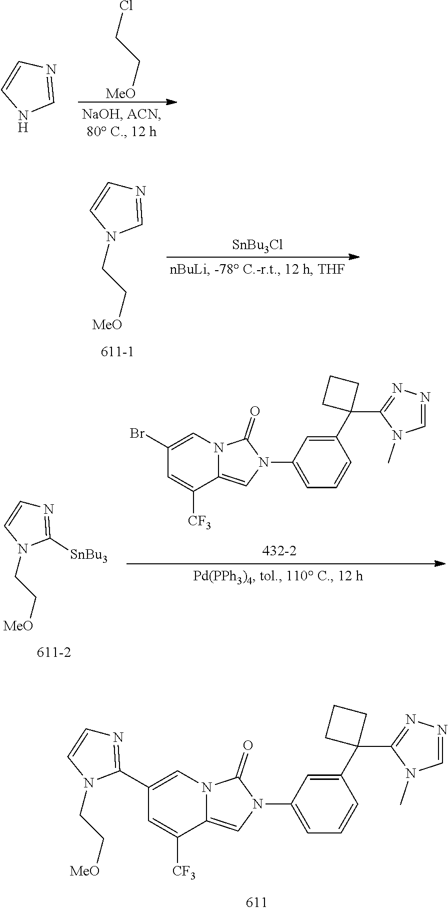

### 1. Synthesis of 611-1

To a solution of imidazole (5 g, 73.45 mmol, 1 equiv) in ACN (150 mL) was added NaOH (58.75 g, 1.47 mol, 20 equiv). The mixture was heated to 80° C. and stirred for 30 min at 80° C. Then 1-chloro-2-methoxy-ethane (6.94 g 73.45 mmol, 1 equiv) was added dropwise to the mixture and stirred for 11.5 h at 80° C. The reaction mixture was diluted with 40 mL 1120 and extracted with DCM:MeOH=10:1 (50 mL×3). The combined organic layers were dried over Na2SO4, filtered and concentrated under reduced pressure to give a residue. The residue was purified by column chromatography (SiO2, Petroleum ether/Ethyl acetate=5/1 to 0/1) to afford 611-1 (6.7 g, 70.8%) as a yellow oil.

1H NMR-611-1: (400 MHz, CHLOROFORM-d) δ 7.51 (s, 1H), 7.04 (s, 1H), 6.97-6.96 (m, 1H), 4.09-4.07 (t, J=8 Hz, 2H), 3.65-3.60 (m, 2H), 3.33 (s, 3H)

### 2. Synthesis of 611-2

To a solution of 611-1 (1 g, 7.93 mmol, 1 equiv) in THF (15 mL) was added dropwise butyllithium (2.5 M, 4.75 mL, 1.5 equiv) at −78° C. The mixture was stirred for 1 h at −78° C. Then tributyl(chloro)stannane (2.83 g, 8.69 mmol, 1.10 equiv) was added dropwise to the reaction mixture. Then the mixture was stirred for 11 h at 15° C. under N2 atmosphere. The resulting solution was concentrated under vacuum. The residue was diluted with 50 mL of hexane and the solids were filtered out. The filtrate was concentrated under vacuum to afford 611-2 (2.8 g, crude) as a yellow oil,

### 3. Synthesis of 611

To a solution of 432-2 (300 mg, 609.40 umol, 1 equiv) in Tol. (10 mL) was added 611-2 (1.52 g, 1.83 mmol, 50% purity, 3 equiv), palladium;triphenylphosphane (70.42 mg, 60.94 umol, 0.1 equiv) under N2. The mixture was heated to 120° C. and stirred for 12 h at 120° C. The reaction mixture was diluted with H2O 15 mL and extracted with EtOAc (10 mL×3). The combined organic layers were washed with brine (20 mL), dried over Na2SO4, filtered and concentrated under reduced pressure to give a residue. The crude product (170 mg) was purified by Prep-HPLC with the following conditions (Column: Phenomenex Luna C18 200*40 mm*10 um; mobile phase: [water (FA)-ACN]; B %: 10%-40%, 8 min; Wave Length: 220 nm; RT 1 (min): 7.5) to afford 611 (66 mg) as a yellow solid.

1H NMR-611: (400 MHz, DMSO-d6) δ 8.36 (s, 1H), 8.16 (s, 0.2H), 7.80 (s, 3H), 7.69-7.64 (m, 1H), 7.53-7.47 (m, 2H), 7.21-7.15 (m, 2H), 7.11 (s, 1H), 4.14 (t, J=8 Hz, 2H), 3.62 (t, J=8 Hz, 2H), 3.22 (d, J=4 Hz, 6H), 2.99-2.90 (m, 2H), 2.75-2.66 (m, 2H), 2.07-1.93 (m, 2H).

### Example 606. Synthesis of Compound 612_P1

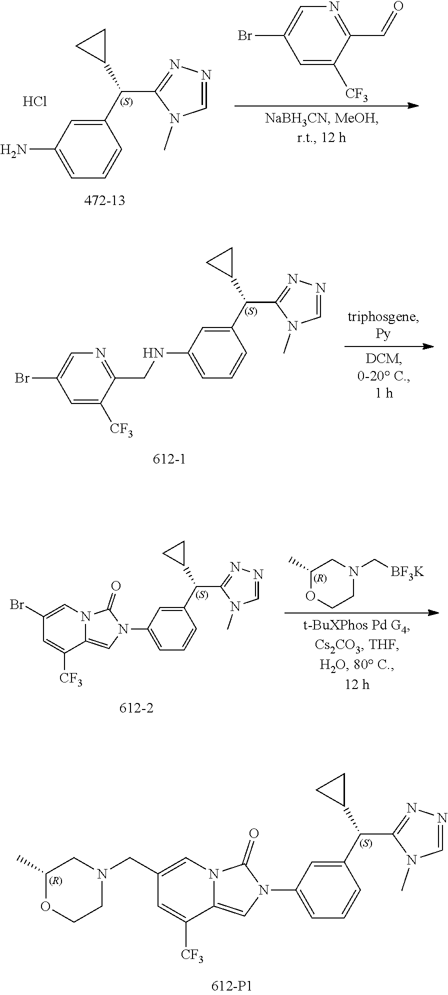

### 1. Synthesis of 612-1

A solution of 472-13 (450 mg, 1.9 mmol, 1.0 equiv) and 5-bromo-3-(trifluoromethyl)-pyridine-2-carbaldehyde (500 mg, 1.9 mmol, 1.0 equiv) in MeOH (10.0 mL) was stirred at 20° C. for 1 hour. Then NaBH3CN (247 mg, 3.8 mmol, 2.0 equiv) was added at 20° C. The resulting mixture was stirred at 20° C. for 11 hours. The reaction was poured into water (30 mL) and the resulting mixture was extracted with EtOAc (2×20 mL) The organic phase was washed with brine (15 mL), dried over anhydrous Na2SO4, concentrated in vacuum to give a residue. The residue was purified by column chromatography (SiO2, DCM: MeOH=20:1 to 1:1) to afford 612-1 (530 mg, 57% yield) as a yellow solid.

### 2. Synthesis of 612-2

To a solution of 612-1 (470 mg, 1.0 mmol, LO equiv) in DCM (2.3 mL) was added dropwise pyrinide (478 ng, 6.0 mmol, 6.0 equiv) at 0° C. After addition, the triphosgene (149 mg, 0.5 equiv) was added at 0° C. The resulting mixture was stirred at 20° C. for 1 hour. The reaction was poured into water (20 mL) and the resulting mixture was extracted with EtOAc (2×15 mL). The organic phase was washed with brine (10 mL), dried over anhydrous Na2SO4, concentrated in vacuum to give a residue. The residue was purified by column chromatography (SiO2, DCM: MeOH=50:1 to 1:1) to afford 612-2 (530 mg, 79% yield) as a yellow solid.

### 3. Synthesis of 612-P1

A mixture of 612-2 (201 mg, 1.0 equiv), potassium hydride;trifluoro-[[(2R)-2-methylmorpholin-4-yl]methyl]boron (270 mg, 12 mmol 3.0 equiv), Cs2CO3 (399 mg, 1.2 mmol, 3.0 equiv), dicyclohexyl-[2-(2,4,6-triisopropylphenyl)phenyl]phosphane;methanesulfonate;[2-[2-(methylamino)phenyl]-phenyl]palladium(1+) (35 mg, 0.1 equiv) in THF (7.0 mL) and H2O (1.7 mL) was degassed and purged with N2 for 3 times. The mixture was stirred at 80° C. for 12 hours under N2 atmosphere. The reaction was poured into water (20 mL) and the resulting mixture was extracted with EtOAc (2×15 mL). The organic phase was washed with brine (10 mL), dried over anhydrous Na2SO4, concentrated in vacuum to give a residue. The residue was purified by prep-HPLC (column: Phenomenex Luna C18 75*30 mm*3 um; mobile phase: [water (FA)-ACN] B %: 15%-45%, 8 min) to afford 612_P1 (113 mg, 50% yield) as a yellow solid. It was further purified by prep-SFC (column: DAICEL CHIRALCEL OD (250 nm*30 nm, 10 um); mobile phase: [0.1NH3H2O IPA]; B %: 25%-45%, 12 min) to afford 612_P1 (71 mg, 54% yield) as a yellow solid.

MS-612_P1: (ES, m/z): [M+H]+ 527.2.

H-NMR-612_P1: (400 MHz, DMSO-d6) δ ppm 8.38 (s, 1H), 7.72-7.62 (m, 3H), 7.54-7.48 (m, 1H), 7.28 (d, J=3.6 Hz, 1H), 7.11 (d, J=3.2 Hz, 2H), 3.85 (d, J=2.4 Hz, 1H), 3.60-3.68 (m, 3H), 3.49 (s, 3H), 3.33 (s, 2H), 2.81 (d, J=2.4 Hz, 1H), 2.73 (d, J=2.8 Hz, 1H), 2.24-2.13 (m, 1H), 1.92-1.82 (m, 1H), 1.75-1.63 (m, 1H), 1.16-1.09 (m, 3H), 0.80-0.72 (m, 1H), 0.71-0.63 (m, 1H), 0.52-0.44 (m, 1H), 0.43-0.35 (m, 1H).

### Example 607. Synthesis of Compound 612_P2

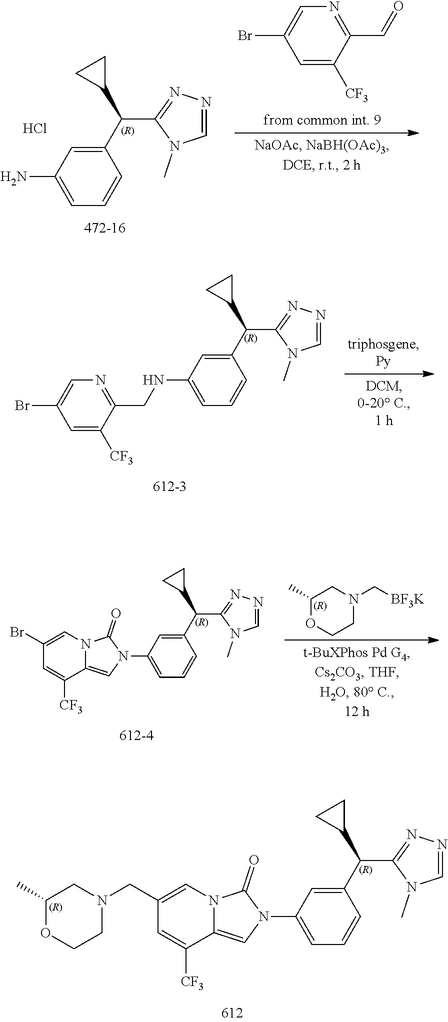

### 1. Synthesis of 612-3

To a solution of 472-16 (550 mg, 2.08 mmol, 1.0 equiv, HCl) and 5-bromo-3-(trifluoroethyl)pyridine-2-carbaldehyde (580 mg, 2.29 mmol, 1.1 equiv) in DCE (15 mL) was added NaOAc (340 mg, 4.15 mmol, 2.0 equiv) at 20° C., After addition, the mixture was stirred at this temperature for 1 hour, and then NaBH(OAc)3 (880 mg, 4.15 mmol, 2.0 equiv) was added at 20° C. The resulting mixture was stirred at 20° C. for 1 hour. The reaction was poured into water (30 ml) and the resulting mixture was extracted with EtOAc (2×20 mL). The organic phase was washed with brine (15 ml), dried over anhdrous Na2SO4, concentrated in vacuum to give a residue. The residue was purified by column chromatography (SiO2, DCM: MeOH=20:1 to 1:1) to afford 612-3 (520 mg, 53%) as a yellow solid.

1H-NMR-612-3: (400 MHz, DMSO-d6) δ 8.96-8.90 (m, 1H), 8.48-8.41 (m, 1H), 8.30-8.25 (m, 1H), 7.03-6.93 (m, 1H), 6.51-6.38 (m, 3H), 6.25-6.16 (m, 1H), 4.50-4.35 (m, 2H), 3.33-3.31 (m, 4H), 1.59-1.47 (m, 1H), 0.57-0.44 (m, 2H), 0.28-0.19 (m, 2H).

### 2. Synthesis of 612-4

To a solution of 612-3 (330 mg, 1.0 equiv) in DCM (5 mL) was added pyridine (30 mg, 6.0 equiv) at 0° C. Then triphosgene (105 mg, 0.5 equiv) was added to the mixture at 0° C. The reaction was stirred for 1 hour at 20° C. The reaction was poured into water (20 mL) and the resulting mixture was extracted with EtOAc (2×15 mL). The organic phase was washed with brine (10 mL), dried over anhydrous Na2SO4, concentrated in vacuum to give a residue. The residue was purified by column chromatography (SiO2, DCM: MeOH 50:1 to 1:1) to afford 612-4 (0.58 g. crude) as a yellow solid.

1H-NMR: (400 MHz, DMSO-d6) δ 8.35-8.32 (m, 1H), 8.03-8.00 (m, 1H), 7.76-7.72 (m, 1H), 7.69-7.63 (m, 1H), 7.49-7.40 (m, 2H), 7.28-7.23 (m, 1H), 7.17-7.13 (m, 1H), 3.62-3.56 (m, 1H), 3.39 (s, 3H), 1.71-1.61 (m, 1H), 0.61-0.47 (m, 2H), 0.39-0.31 (m, 2H).

### 3. Synthesis of 612_P2

A mixture of 612-4 (210 mg, 1.0 equiv), potassium hydride;trifluoro-[[(2R)-2-methylmorpholin-4-yl]methyl]boron (235 mg, 1.07 mmol, 2.5 equiv), Xphos Pd G4 (36 mg, 0.1 equiv) and Cs2CO3 (416 mg, 1.28 mmol 3.0 equiv) in THF (5 mL)/H2O (1 mL) was degassed and purged with N2 for 3 times. The mixture was stirred at 80° C. for 12 hours under N2 atmosphere. The reaction was poured into water (20 mL) and the resulting mixture was extracted with EtOAc (2×15 ml). The organic phase was washed with brine (10 mL), dried over anhydrous Na2SO4, concentrated in vacuum to give a residue. The residue was purified by column chromatography (SiO2, DCM: MeOH=50:1 to 1:1) to give crude product. The crude product (300 mg) was further purified by prep-HPLC (column: Phenomenex Luna C18 200*40 mm*10 um; Mobile phase A: water (FA), Mobile phase B: ACN; Flow rate: 70 mL/min; Gradient: 5% B to 40% B in 8 min; Wave Length: 220 nm) to afford 612_P2 (99.7 mg, 44%) as a yellow solid. It was further purified by prep-SFC (column: DAICEL CHIRALCEL OD (250 nm 30 mm, 10 um); Mobile phase: [Neu-IPA]; B %: 25%-47%, 14 min, Flow rate: 70 mL/min; Wave Length: 220/254 am) to afford 612_P2 (61.2 mg, 62%) as a yellow solid.

MS: (ES, m/z): [M+H]+ 527.55. 1H-NMR: (400 MHz, METHANOL-d4) δ 8.43-8.38 (m, 1H), 7.73-7.68 (m, 2H), 7.68-7.64 (m, 1H), 7.56-7.50 (m, 1H), 7.33-7.27 (m, 1H), 7.17-7.13 (m, 1H), 7.12-7.10 (m, 1H), 3.91-3.83 (m, 1H), 3.75-3.61 (m, 3H), 3.53-3.47 (m, 3H), 3.37 (s, 2H), 2.88-2.73 (m, 2H), 2.27-2.17 (m, 1H), 1.96-1.86 (m, 1H), 1.78-1.66 (m, 1H), 1.18-1.11 (m, 3H), 0.83-0.63 (m, 2H), 0.55-0.38 (m, 2H).

### Example 608. Synthesis of Compound 613

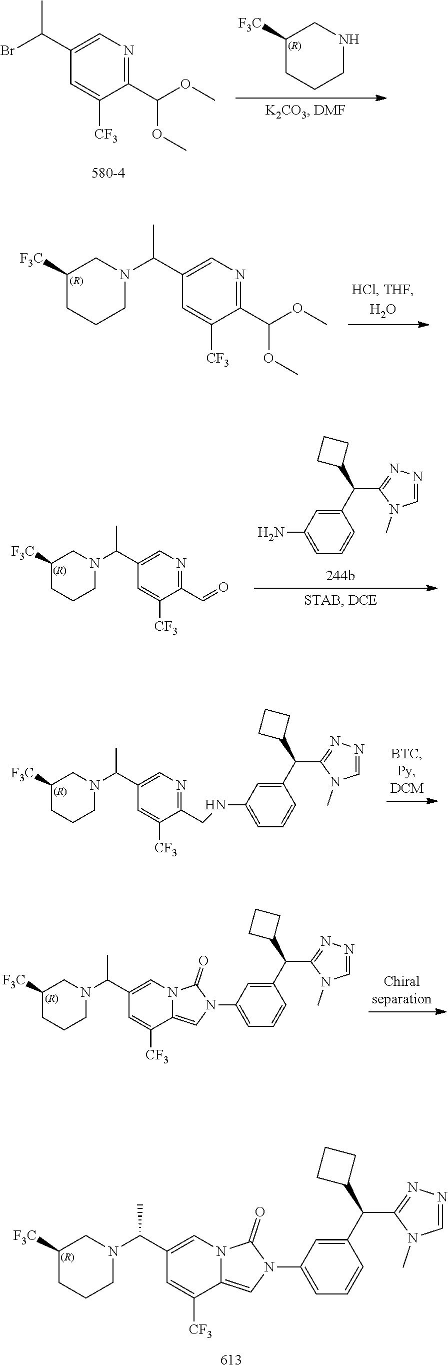

### 1. Synthesis of 613-1

Into a 40 mL sealed tube were added 580-4 (886.79 mg, 5.790 mmol, 2 equiv), DMF (10 mL) and K2CO3 (1200.39 mg, 8.685 mmol, 3 equiv) at room temperature. To the above mixture was added (R)5-(1-bromoethyl)-2 (dimethoxymethyl)3-trifluoromethyl) pyridine (950 mg, 2.895 mmol, 1 equiv) at room temperature. The resulting mixture was stirred for additional overnight at 80° C. The reaction was quenched with sat. NH4Cl (aq.) (50 mL) at room temperature. The resulting mixture was extracted with EtOAc (3×50 mL). The combined organic layers were concentrated under reduced pressure. The residue was purified by Prep-TLC (CH₂Cl₂/MeOH 40:1) to afford 613-1 (800 mg, 65.57%) as a light yellow oil.

### 2. Synthesis of 613-2

Into a 40 mL sealed tube were added 613-1 (800 mg, 1.998 mmol, 1 equiv), (1 M) HCl (8 mL) and THF (2 mL) at room temperature. The resulting mixture was stirred for 2 h at 80° C. The mixture was basified to pH 7 with saturated NaHCO₃ (aq.) (30 mL). The resulting mixture was extracted with EtOAc (3×30 mL). The combined organic layers were concentrated under reduced pressure. The residue was purified by Prep-TLC (CH2Cl2/MeOH 40:1) to afford 613-2 (700 mg, 93.93%) as a light yellow oil.

### 3. Synthesis of 613-3

Into a 40 mL sealed tube were added 613-2 (450 mg, 1.270 mmol, 1 equiv), DCE (6 mL), 244b (615.57 mg, 2.540 mmol, 2 equiv) and STAB (807.57 mg, 3,810 mmol, 3 equiv) at room temperature. The resulting mixture was stirred for 1 h at room temperature under nitrogen atmosphere. The reaction was quenched with water (20 mL) at room temperature. The resulting mixture was extracted with CH2Cl2 (3×20 mL). The combined organic layers were concentrated under reduced pressure. The residue was purified by Prep-TLC (CH2Cl2/MeOH 20:1) to afford 613-3 (500 mg, 64.41%) as a light yellow oil.

### 4. Synthesis of 613-4

Into a 100 mL round-bottom flask were added 613-3 (450 mg, 0,775 mmol, 1 equiv), DCM (20 mL) and pyridine (429.13 mg, 5.425 mmol, 7 equiv) at 0° C. To the above mixture was added triphosgene (114.99 mg, 0.388 mmol, 0.50 equiv) at 0° C. The resulting mixture was stirred for additional 10 min at 0° C. The was basified to pH 8 with saturated NaHCO₃ (aq.) (40 mL). The resulting mixture was extracted with CH2Cl2 (3×40 mL). The combined organic layers were concentrated under reduced pressure. The residue was purified by Prep-TLC (CH2Cl2/MeOH 16:1) to afford crude product (350 mg). The crude product (350 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Plow rate: 60 mL/min; Gradient: 55% B to 72% B in 7 min; Wave Length: 220 nm; RT1 (min): 6.97.) to afford 613-3 (120 mg, 22.97%) as a light yellow solid.

### 5. Synthesis of 613

The 613-4 (200 mg) was purified by Chiral separation with the following conditions (Column: (R, R)-WHELK-O1-Kromasi, 5*25 cm, 5 μm; Mobile Phase A: Hex (0.5% 2M NH3-MeOH)-HPLC, Mobile Phase B: EtOH:DCM=1:1-HPLC; Flow rate: 20 mL/min; Gradient: 30% B to 30% B in 28 min; Wave Length: 220/254 nm; RT1 (min): 16.32; RT2 (min): 22.55; The first peak was the product. Sample Solvent: EtOH:DCM=1:1-HPLC; Injection Volume: 0.7 mL; Number of Runs: 4) to afford 613 (58.6 mg, 28.98%) as a light yellow solid,

LCMS: (ES, m/z): [M+H]+ 607. H-NMR18: (300 MHz, CD3OD, ppm): δ 1.30-1.77 (m, 6H), δ 1.80-1.83 (m, 2H), δ 1.90-1.97 (m, 5H), δ 2.05-2.11 (m, 1H), δ 2.22-2.25 (d, 2H), δ 2.27 (S, 1H), δ 2.88-2.91 (m, 1H), δ 3.05-3.07 (m, 1H), δ 3.53-3.57 (m, 4H), δ 4.28-4.31 (m, 1H), δ 7.12-7.16 (m, 2H), δ 7.26-7.28 (m, 1H), δ 7.48-7.52 (m, 1H), δ 7.64-7.67 (m, 3H), δ 8.37 (s, 1H).

### Example 609. Synthesis of Compound 614

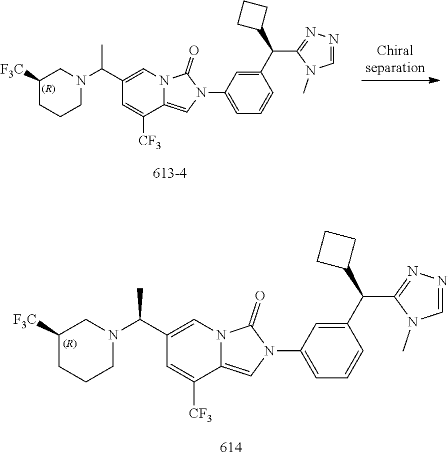

### 1. Synthesis of 614

The 613-4 (200 mg) was purified by Chiral separation with the following conditions (Column: (R, R)-WHELK-O1-Kromasi, 5*25 cm, 5 p m; Mobile Phase A: Hex (0.5% 2M NH3-MeOH)-HPLC, Mobile Phase B: EtOH:DCM=1:1-HPLC; Flow rate: 20 mL/min; Gradient: 30% B to 30% B in 28 min; Wave Length: 220/254 am; RT1 (min): 16.32; RT2 (min): 22.55; The second peak was the product. Sample Solvent: EtOH:DCM=1:1-HPLC; Injection Volume: 0.7 mL; Number of Runs: 4) to afford 614 (31.7 mg, 15.36%) as a light yellow solid.

LCMS 72-614: (ES, m/z): [M+H]+ 607

H-NMR18-614: (300 MHz, CD3OD, ppm): δ 1.30-1.77 (m, 6H), δ 1.80-1.83 (m, 2H), δ 1.90-2.09 (m, 5H), δ 2.12-2.25 (m, 3H), δ 2.27 (d, 1H), δ 2.90-2.93 (m, 2H), δ 3.10-3.29 (m, 1H), δ 3.53-3.55 (m, 4H), δ 4.28-4.31 (m, 1H), δ 7.12-7.14 (m, 2H), δ 7.26-7.28 (m, 1H), δ 7.48-7.52 (m, 1H), δ 7.64-7.67 (m, 3H), δ 8.37 (s, 1H).

### Example 610. Synthesis of Compound 615

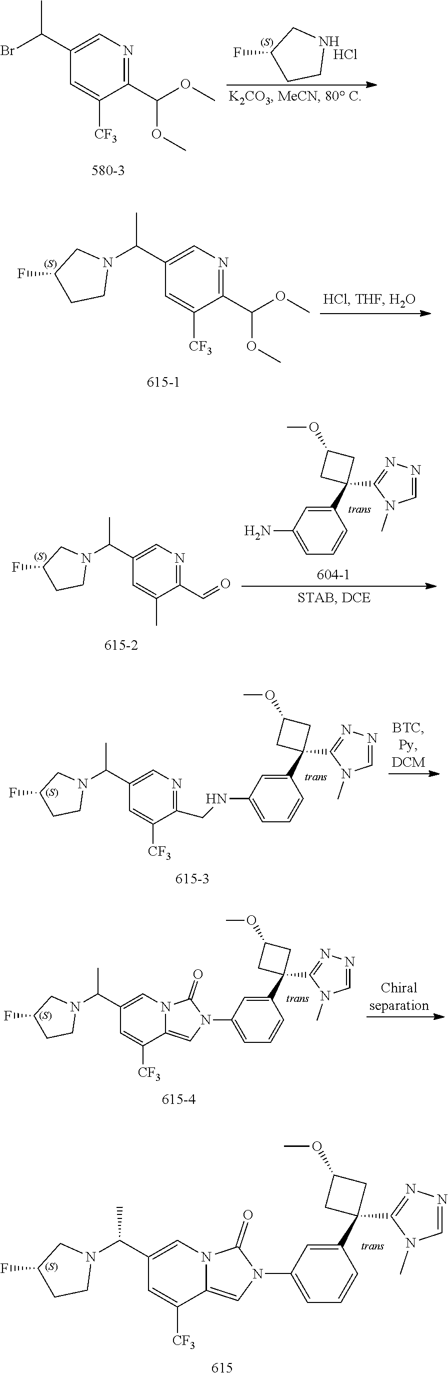

### 1. Synthesis of 615-1

A solution of 580-3 in DCM (8 mL) in MeCN (12 mL) was treated with(S)-3-fluoropyrrolidine hydrochloride (0.81 g, 9.143 mmol, 2.5 equiv) and K₂CO₃ (1.52 g, 10.971 mmol, 3 equiv) at room temperature. The resulting mixture stirred for 4 h at 80° C. under nitrogen atmosphere. The reaction was quenched with sat. NH₄Cl (aq.) (30 mL) at room temperature. The resulting mixture was extracted with CH₂Cl₂ (3×20 mL). The combined organic layers were concentrated under reduced pressure. The residue was purified by Prep-TLC (PE/EA 2:1) to afford 615-1 (1.1 g, 78.70%) as a light yellow oil.

### 2. Synthesis of 615-2

A solution of 615-1 (1.1 g, 3.271 mmol, 1 equiv) in HCl (5.5 mL, 1M) and THF (5.5 mL) was stirred for 2 h at 80° C. The mixture was basified to pH 8 with saturated NaHCO₃ (aq.) (10 ml). The resulting mixture was extracted with CH₂Cl₂ (3×30 mL). The combined organic layers were concentrated under reduced pressure. The residue was purified by Prep-TLC (PE/EA 2:1) to afford 615-2 (750 mg, 79.00%) as a yellow oil.

### 3. Synthesis of 615-3

A solution of 615-2 (375 mg, 1.292 mmol, 1 equiv) in DCE (4 mL) was treated with 604-1 (333.74 mg, 1.292 mmol, 1 equiv) for overnight at room temperature under nitrogen atmosphere followed by the addition of STAB (547.63 mg, 2.584 mmol, 2 equiv) at room temperature. The resulting mixture was stirred for 4 h at room temperature under nitrogen atmosphere. The reaction was quenched with sat. NaHCO₃ (aq.)(20 ml). The resulting mixture was extracted with CH₂Cl₂ (3×15 mL). The combined organic layers were concentrated under reduced pressure. The residue was purified by Prep-TLC (CH₂Cl₂/MeOH 16:1) to afford 615-3 (388 mg, 55.26%) as a yellow solid.

### 4. Synthesis of 615-4

A solution of 615-3 (380 mg, 0,713 mmol, 1 equiv) in DCM (12 mL) was treated with Pyridine (338.63 mg, 4.278 mmol, 6 equiv) at room temperature under nitrogen atmosphere followed by the addition of Triphosgene (84.69 mg, 0.285 mmol, 0.4 equiv) at 0° C. The resulting mixture was stirred for 20 min at 0° C. under nitrogen atmosphere. The reaction was quenched with sat. NaHCO₃ (aq.)(30 ml) at room temperature. The resulting mixture was extracted with CH₂Cl₂/MeOH (10:1) (3×20 mL). The combined organic layers were concentrated under reduced pressure. The residue was purified by Prep-TLC (CH2Cl₂/MeOH 18:1) to afford 615-4 (380 mg, 93.44%) as a yellow solid.

### 5. Synthesis of 615

615-4 (370 mg) was purified by Prep-Chiral-HPLC with the following conditions (Column: CHIRALPAK IF, 2*25 cm, 5 μm; Mobile Phase A: MtBE (0.5% 2M NH3-MeOH), Mobile Phase B: EtOH:DCM=1:1-; Flow rate: 20 mL/min; Gradient: 25% B to 25% B in 19 min; Wave Length: 220/254 nm; RT1 (min): 12.40; RT2 (min): 15.02; the first peak was the product) to afford 615 (90.9 mg, 23.92%) as a yellow solid,

LCMS-615: (ES, m/z): [M+H]⁺ 553

NMR-615: (400 MHz, dmso-d6, δ ppm): 1.28-1.30 (d, 3H), 1.77-1.87 (m, 1H), 2.03-2.08 (m, 1H), 2.39-2.41 (m, 1H), 2.59-2.68 (m, 2H), 2.77-2.85 (m, 2H), 2.89-2.92 (m, 1H), 3.08-3.13 (m, 2H), 3.16 (s, 3H), 3.23 (s, 3H), 3.26-3.29 (m, 1H), 4.06-4.09 (t, 1H), 5.14-5.29 (m, 1H), 7.06 (s, 1H), 7.25-7.27 (d, 1H), 7.41 (s, 1H), 7.49-7.52 (t, 1H), 7.65-7.71 (m, 2H), 7.77 (s, 1H), 8.34 (s, 1H).

### Example 611. Synthesis of Compound 616

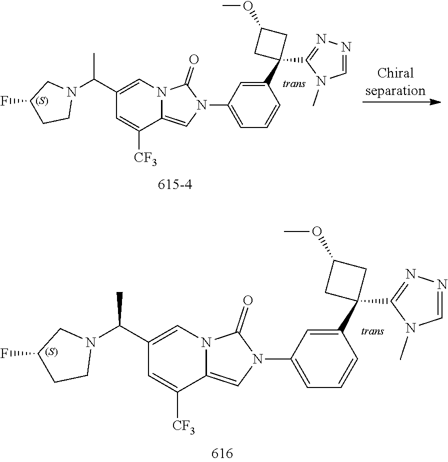

### 1. Synthesis of 616

The 615-4 (370 mg) was purified by Prep-Chiral-HPLC with the following conditions (Column: CHIRALPAK IF, 2*25 cm, 5 μm; Mobile Phase A: MtBE (0.5% 2M NH3-MeOH), Mobile Phase B: EtOH:DCM=1:1; Flow rate: 20 mL/in; Gradient: 25% B to 25% B in 19 min; Wave Length: 220/254 nm; RT1 (min): 12.40; RT2 (min): 15.02; the second peak was product) to afford 616 (119.6 mg, 32.32%) as a yellow solid.

LCMS: (ES, m/z): [M+H]⁺553. NMR: (400 MHz, DMSO, δ ppm): 1.28-1.30 (d, 3H), 1.77-1.95 (m, 1H), 2.03-2.18 (m, 1H), 2.20-2.27 (m, 1H), 2.56-2.66 (m, 1H), 2.73-2.83 (m, 3H), 2.88-2.92 (m, 1H), 3.08-3.13 (m, 2H), 3.17 (s, 3H), 3.24 (s, 3H), 3.26-3.28 (m, 1H), 4.04-4.08 (t, 1H), 5.11-5.26 (m, 1H), 7.06 (s, 1H), 7.24-7.26 (d, 1H), 7.41 (s, 1H), 7.48-7.52 (t, 1H), 7.66-7.72 (m, 2H), 7.77 (s, 1H), 8.34 (s, 1H).

### Example 612. Synthesis of Compound 617

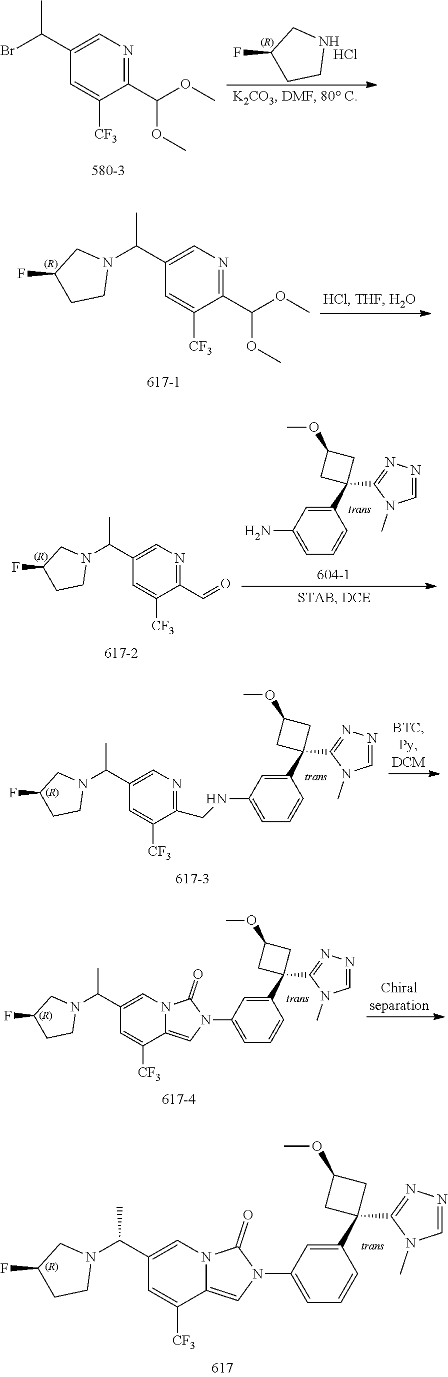

### 1. Synthesis of 617-1

A solution of 580-3 in DMF (20 mL) was treated with (3R)-3-fluoropyrrolidine (1.32 g, 14.780 mmol, 2.5 equiv) and K₂CO₃ (2.45 g, 17.736 mmol, 3 equiv). The resulting mixture was stirred for 4 h at 80° C. under nitrogen atmosphere. The reaction was quenched with sat. NH₄Cl (aq.) (20 mL) at room temperature. The resulting mixture was extracted with CH₂?Cl2 (3×20 mL). The combined organic layers were concentrated under reduced pressure. The residue was purified by Prep-TLC (PE/EA 2:1) to afford 617-2 (1.7 g, 77.80%) as a light yellow oil

### 2. Synthesis of 617-2

A solution of 617-1 (1.7 g, 5,055 mmol, 1 equiv) in HCl (8.5 mL, 1M) and THF (8.5 mL) was stirred for 2 h at 80° C. under nitrogen atmosphere. The mixture was basified to pH 8 with saturated NaHCO₃ (aq.) (30 mL) The resulting mixture was extracted with EtOAc (3×40 mL). The combined organic layers were concentrated under reduced pressure. The residue was purified by Prep-TLC (PE/EA 2:1) to afford 617-2 (800 mg, 49.07%) as a yellow oil.

### 3. Synthesis of 617-3

A solution of 617-2 (400 mg, 1.378 mmol, 1 equiv) in DCE (4 mL) was treated with 604-1 (355.99 mg, 1.378 mmol, 1 equiv) for overnight at room temperature under nitrogen atmosphere followed by the addition of STAB (584.13 mg, 2.756 mmol, 2 equiv) at room temperature. The resulting mixture was stirred for 4 h at room temperature. The reaction was quenched with sat. NH4Cl (aq.) (20 mL) at room temperature. The resulting mixture was extracted with CH2Cl2 (3×20 mL). The combined organic layers were concentrated under reduced pressure. The residue was purified by Prep-TLC (CH₂Cl₂/MeOH 16:1) to afford 617-3 (500 mg, 66.76%) as a yellow solid.

### 4. Synthesis of 617-4

A solution of 617-3 (500 mg, 0.939 mmol, 1 equiv) in DCM (15 mL) was treated with Pyridine (445.56 mg, 5.634 mmol, 6 equiv) at room temperature under nitrogen atmosphere followed by the addition of Triphosgene (111.43 mg, 0.376 mmol, 0.4 equiv) at 0° C. The resulting mixture was stirred for 20 min at 0° C. under nitrogen atmosphere. The reaction was quenched with sat. NaHCO₃ (aq.) (30 ml) at room temperature. The resulting mixture was extracted with CH₂Cl₂/MeOH (10:1) (3×20 mL). The combined organic layers were concentrated under reduced pressure. The residue was purified by Prep-TLC (CH2Cl₂/MeOH 18:1) to afford 617-4 (390 ng, 72.88%) as a yellow solid.

### 5. Synthesis of 617-0

The 617-4 (390 mg) was purified by Prep-Chiral-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1; Flow rate: 20 mL/min; Gradient: 40% B to 40% B in 20 min; Wave Length: 220/254 nm; RT₁ (min): 15.55; RT2 (min): 17.97; the first peak was the product) to afford 617 (113.3 mg, 29.05%) as a yellow solid.

LCMS-617: (ES, m/z): [M+H]⁺ 559

NMR-617: (400 MHz, DMSO, δ ppm): 1.23-1.30 (m, 3H), 1.72-1.92 (m, 1H), 2.03-2.30 (m, 2H), 2.51-2.55 (m, 2H), 2.67-2.72 (m, 2H), 2.83-2.89 (m, 1H), 3.16 (s, 3H), 3.24 (s, 3H), 3.26-3.30 (m, 3H), 3.82-3.85 (m, 1H), 5.12-5.25 (m, 1H), 7.06-7.09 (m, 2H), 7.42 (s, 1H), 7.46-7.50 (t, 1H), 7.65-7.71 (m, 3H), 8.34 (s, 1H).

### Example 613. Synthesis of Compound 618

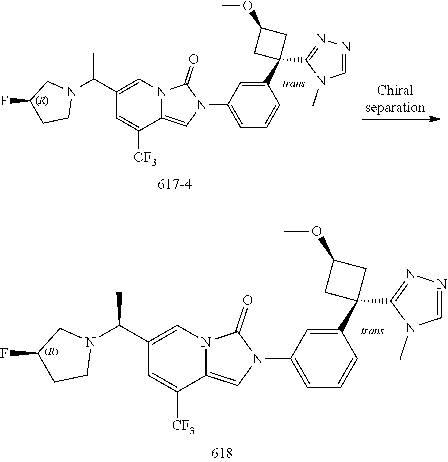

### 1. Synthesis of 618

The 617-4 (390 mg) was purified by Prep-Chiral-HPLC with the following conditions (Column: CHIRALPAK IC, 2*25 cm, 5 pnm; Mobile Phase A: Hex (0.5% 2M NH₃-MeOH), Mobile Phase B: EtOH:DCM=1:1; Flow rate: 20 mL/min; Gradient: 40% B to 40% B in 20 min; Wave Length: 220/254 nm; RT₁ (min): 15.55; RT₂ (min): 17.97; the second peak was product) to afford 618 (120 mg, 30.77%) as a yellow solid.

LCMS-618: (ES, m/z): [M+H]⁺ 559

NMR-618: (400 MHz, DMSO, δ ppm): 1.23-1.30 (m, 3H), 1.72-1.95 (m, 1H), 2.03-2.30 (m, 1H), 2.32-2.45 (m, 2H), 2.67-2.72 (m, 2H), 2.80-2.96 (m, 1H), 3.16-3.21 (m, 7H), 3.24 (s, 2H), 3.25-3.30 (m, 2H), 3.82-3.88 (m, 1H), 5.16-5.28 (m, 1H), 7.08-7.10 (m, 2H), 7.22-7.35 (m, 1H), 7.42 (s, 1H), 7.46-7.50 (t, 1H), 7.65-7.72 (m, 2H), 8.38 (s, 1H).

### Example 614. Synthesis of Compound 619

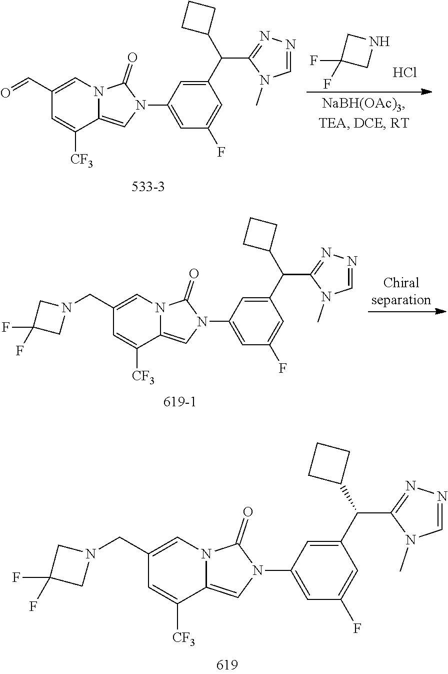

### 1. Synthesis of 619-1

To a stirred solution of 533-3 (600 mg, 1.267 mmol, 100 equiv) and 3,3-difluoroazetidine hydrochloride (246.24 mg, 1.900 mmol, 1.5 equiv) in DCE (10 mL) were added TEA (256.49 mg, 2,534 mmol, 2 equiv) at room temperature. The resulting mixture was stirred for 1 h at room temperature. To the above mixture was added STAB (537.20 mg, 2.534 mmol, 2 equiv) at room temperature. The resulting mixture was stirred overnight at room temperature. The reaction was quenched with saturated NH4Cl (aq.) (50 mL) at room temperature. The resulting mixture was extracted with CH2Cl2 (3×30 mL). The resulting mixture was concentrated under reduced pressure. The crude product was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 30% B to 41% B in 7 min, Wave Length: 254; 220 nm; RT1 (min): 5.92) to afford 619-1 (150 mg, 21.07%) as a yellow solid.

### 2. Synthesis of 619

The 619-4 (150 mg) was purified by Chiral separation with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min: Gradient: 40% B to 40% B in 18.5 min; Wave Length: 220/254 nm; RT1 (min): 12.76; RT2 (min): 16.08; the first peak is product) to afford 619 (25.3 mg, 16.75%) as a yellow solid.

LC-MS-619: (ES, m/z): [M+H]+ 551

H-NMR-619: (400 MHz, DMSO-d6, δ ppm): 1.62-1.65 (m, 1H), 1.66-1.85 (m, 4H), 2.09-2.10 (d, 1H), 3.15-3.24 (m, 1H), 3.46 (s, 3H), 3.52 (s, 2H), 3.64-3.70 (m, 4H), 3.30-3.33 (d, 1H), 7.02-7.06 (m, 2H), 7.43 (s, 1H), 7.68-7.70 (d, 2H), 7.77 (s, 1H), 8.35 (s, 1H).

### Example 615. Synthesis of Compound 620

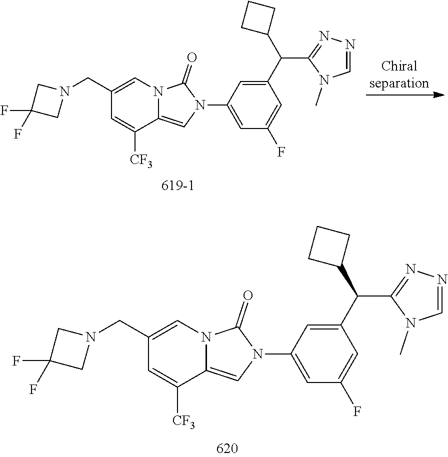 [SMILES: Cn1cnnc1C(c1cc(F)cc(-n2cc3c(C(F)(F)F)cc(CN4CC(F)(F)C4)cn3c2=O)c1)C1CCC1 ; Cn1cnnc1[C@@H](c1cc(F)cc(-n2cc3c(C(F)(F)F)cc(CN4CC(F)(F)C4)cn3c2=O)c1)C1CCC1]

### 1. Synthesis of 620

The 619-1 (150 mg) was purified by Chiral separation with the following conditions (Column: CHIRALPAK IC, 2*25 cm, 5 μm; Mobile Phase A: Hex (0.5% 2M NH3-MeOH), Mobile Phase B: EtOH:DCM=1:1; Flow rate: 20 mL/min; Gradient: 40% B to 40% B in 18.5 min; Wave Length: 220/254 nm; RT1 (min): 12.76; RT2 (min): 16.08; the second peak is product) to afford 619 (26.3 mg, 17.36%) as a yellow solid.

LC-MS: (ES, m/z): [M+H]+ 551. H-NMR: (400 MHz, DMSO-d6, δ ppm): 1.67-1.69 (m, 1H), 1.70-1.85 (m, 4H), 2.05-2.08 (m, 1H), 3.17-3.19 (m, 1H), 3.46 (s, 3H), 3.52 (s, 2H), 3.60-3.69 (m, 4H), 4.30-4.33 (d, 1H), 7.02-7.06 (m, 2H), 7.43 (s, 1H), 7.68-7.71 (m, 2H), 7.77 (s, 1H), 8.35 (s, 1H).

### Example 616. Synthesis of Compound 621

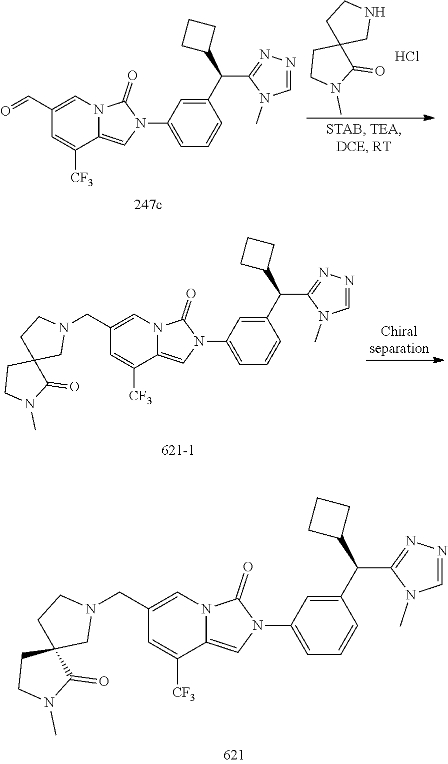

### 1. Synthesis of 621-1

To a stirred solution of 247c (500 mg, 1.098 mmol, 1 equiv) and 2-methyl-2,7-diazaspiro[4.4]nonan-1-one hydrochloride (418.65 mg, 2196 mmol, 2 equiv) in DCE (20 mL) were added TEA (222.19 mg, 2.196 mmol, 2 equiv) at room temperature. The resulting mixture was stirred for 3 h at room temperature. To the above mixture was added STAB (1.86 g, 8.782 mmol, 2 equiv) at room temperature. The resulting mixture was stirred overnight at room temperature. The reaction was quenched with water (100 mL) at room temperature. The aqueous layer was extracted with DCM (2×50 mL). The resulting mixture was concentrated under vacuum. The residue was purified by reverse flash chromatography with the following conditions: column, C18 silica gel; mobile phase, MeCN in Water (10 mmol/L NH4HCO3), 10% to 50% gradient in 10 min; detector, UV 254 nm. This resulted in 621-1 (320 mg, 47.63%) as a yellow solid.

### 2. Synthesis of 621

The 621-1 (320 mg) was purified by Prep-Chiral-HPLC with the following conditions (Column: CHIRALPAK IG, 2*25 cm, 5 μm; Mobile Phase A: Hex (0.5% 2M N1-MeOH), Mobile Phase B: EtOH:DCM=1:1; Flow rate: 20 mL/min; Gradient: 50% B to 50% B in 16 min; Wave Length: 220/254 um; RT1 (min): 8.91; RT2 (min): 12.05; the first peak is product) to afford 621 (128.5 mg, 38.43%) as a yellow solid.

LC-MS: (ES, m/z): [M+H]⁺ 594. H-NMR: (400 MHz, DMSO-d6, δ ppm): 1.65-1.92 (m, 6H), 1.93-2.16 (m, 4H), 2.44-2.50 (m, 2H), 2.61-2.63 (m, 1H), 2.72 (s, 3H), 2.78-2.82 (m, 1H), 3.19-3.25 (m, 3H), 3.43 (s, 5H), 4.25-4.28 (d, 1H), 7.04 (s, 1H), 7.18-7.20 (d, 1H), 7.32 (s, 1H), 7.42-7.46 (m, 1H), 7.69-7.74 (m, 3H), 7.73 (s, 1H), 8.33 (s, 1H).

### Example 617. Synthesis of Compound 622

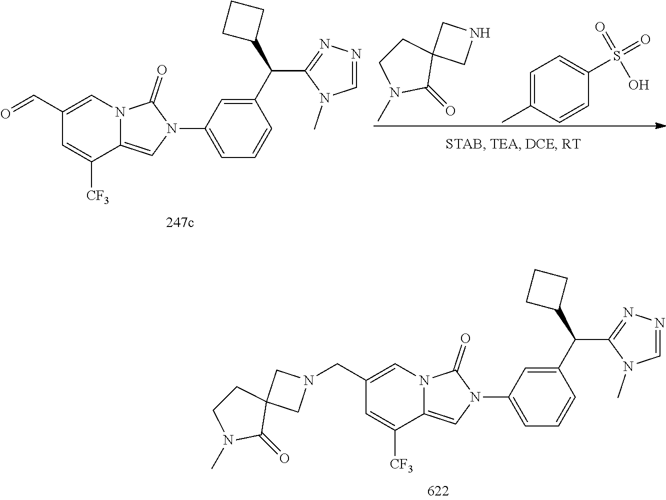

### 1. Synthesis of 622

To a stirred solution of 247c (400 mg, 0.878 mmol, 1.00 equiv) and 6-methyl-2,6-diaza-spiro[3.4]octan-5-one tosylate (411.53 mg, 1,317 mmol, 1.5 equiv) in DCE (5 mL) were added TEA (177.75 mg, 1.756 mmol, 2 equiv) at room temperature. The resulting mixture was stirred for 1 h at room temperature. To the above mixture was added STAB (372.28 mg, 1.756 mmol, 2 equiv) at room temperature. The resulting mixture was stirred for overnight at room temperature. The reaction was quenched by the addition of NH4Cl (aq.) (80 mL) at room temperature. The resulting mixture was extracted with CH2Cl2 (3×40 mL). The resulting mixture was concentrated under reduced pressure. The residue was purified by reverse flash chromatography with the following conditions: column, C18 silica gel; mobile phase, MeCN in Water (10 mmol/L N14HCO3), 50% to 60% gradient in 10 min; detector, UV 254 nm. This resulted in 622 (122.0 mg, 23.22%) as a yellow solid.

LC-MS-622: (ES, m/z): [M+H]+ 580

H-NMR-622: (400 MHz, DMSO-d6, δ ppm): 1.69-1.84 (m, 5H), 2.07-2.09 (m, 1H), 2.25-2.34 (m, 2H), 2.75 (s, 3H), 3.15-3.26 (m, 7H), 3.31 (s, 2H), 3.43 (s, 3H), 4.24-4.27 (d, 1H), 6.99 (s, 1H), 7.18-7.20 (d, 1H), 7.30 (s, 1H), 7.41-7.45 (m, 1H), 7.65-7.69 (m, 1H), 7.74 (s, 1H), 8.32 (s, 1H).

### Example 618. Synthesis of Compound 623

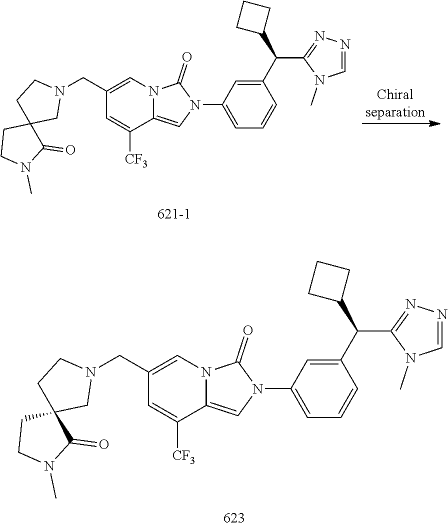 [SMILES: CN1CCC2(CCN(Cc3cc(C(F)(F)F)c4cn(-c5cccc([C@H](c6nncn6C)C6CCC6)c5)c(=O)n4c3)C2)C1=O ; CN1CC[C@@]2(CCN(Cc3cc(C(F)(F)F)c4cn(-c5cccc([C@H](c6nncn6C)C6CCC6)c5)c(=O)n4c3)C2)C1=O]

### 1. Synthesis of 623

The 621-1 (320 mg) was purified by Prep-Chiral-HPLC with the following conditions (Column: CHIRALPAK IG, 2*25 cm, 5 um; Mobile Phase A: Hex (0.5% 2M NH3-MeOH), Mobile Phase B: EtOH:DCM=1:1; Flow rate: 20 mL/min; Gradient: 50% B to 50% B in 16 min; Wave Length: 220/254 nm; RT1 (min): 8.91; RT2 (min): 12.05; the second peak is product) to afford 623 (110.1 mg, 34.03%) as a yellow solid.

LC-MS-623: (ES, m/z): [M+H]+ 594

H-NMR-623: (400 MHz, DMSO-d6, δ ppm): 1.65-1.92 (m, 6H), 1.93-2.16 (m, 4H), 2.44-2.50 (m, 2H), 2.61-2.63 (m, 1H), 2.72 (s, 3H), 2.78-2.82 (m, 1H), 3.19-3.25 (m, 3H), 3.43 (s, 5H), 4.25-4.28 (d, 1H), 7.04 (s, 1H), 7.18-7.20 (d, 1H), 7.32 (s, 1H), 7.42-7.46 (m, 1H), 7.69-7.74 (m, 3H), 7.73 (s, 1H), 8.33 (s, 1H).

### Example 619. Synthesis of Compound 624

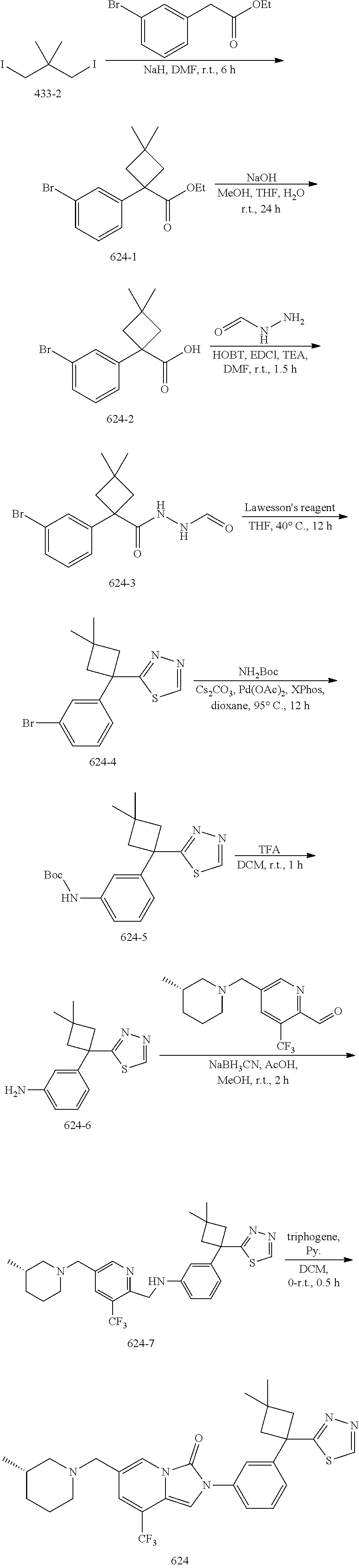

### 1. Synthesis of 624-1

To a solution of 433-2 (12 g, 37 mmol, 0.9 equiv) and ethyl 2-(3-bromophenyl)acetate (10 g, 41.14 mmol, 1 equiv) in DMF (120 mL) was added NaI (3.29 g, 82.3 mmol, 60% purity, 2 equiv), the mixture was stirred at 20° C. for 6 hr. The reaction mixture was diluted with water (250 mL), extracted with EtOAc (150 mL×3). The combined organic layers were dried over anhydrous Na2SO4, filtered and concentrated in vacuum. The residue was purified by flash silica gel chromatography eluted with EA/PE=1:10 to afford 624-1 (7.5 g, 59%) as a colorless oil,

1H-NMR-624-1: (400 MHz, CHLOROFORM-d) δ 7.37 (s, 1H), 7.30-7.24 (m, 1H), 7.18-7.07 (m, 2H), 4.06-3.97 (m, 2H), 2.67 (d, J=12.8 Hz, 2H), 2.24 (d, J=12.8 Hz, 2H), 1.10 (t, J=7.2 Hz, 3H), 1.07 (s, 3H), 0.94 (s, 3H)

### 2. Synthesis of 624-2

To a solution of ethyl 624-1 (5.5 g, 17.6 mmol, 1 equiv) in MeOH (55 mL) and THF (14 mL) and H2O (14 mL) was added NaOH (1.41 g, 35.3 mmol, 2 equiv). The mixture was stirred at 20° C. for 24 hr. The reaction mixture was concentrated in vacuum to remove MeOH (55 mL) and THF (14 mL). Then the mixture was diluted with water (30 mL), adjusted pH to 2 by 1H HCl. The mixture was filtered and the filter cake was concentrated in vacuum to afford 624-2 (4.6 g, 92%) as a white solid.

1H-NMR-624-2: (400 MHz, METHANOL-d4) δ 7.46 (t, J=2.0 Hz, 1H), 7.38-7.33 (m, 1H), 7.32-7.28 (m, 1H), 7.25-7.19 (m, 1H), 2.79-2.67 (m, 2H), 2.30 (d, J=12.8 Hz, 2H), 1.16 (s, 3H), 1.00 (s, 3H)

### 3. Synthesis of 624-3

To a solution of 624-2 (5.5 g, 19.4 mmol, 1 equiv) and formohydrazide (1.75 g, 29.1 mmol, 1.5 equiv) in DMF (55 mL) was added EDCI (5.59 g, 29.1 mmol, 1.5 equiv), HOBt (3.94 g, 29.1 mmol, 1.5 equiv) and TEA (8.11 mL, 58.27 mmol, 3 equiv) tinder nitrogen atmosphere. The mixture was stirred at 20° C. for 1.5 h under nitrogen atmosphere. The reaction mixture was diluted with Sat.NH4Cl (100 mL), extracted with EtOAc (50 mL×3). The combined organic layers were dried over anhydrous Na2SO4, filtered and concentrated in vacuum. The residue was purified by flash silica gel chromatography eluted with EA/PE=1:3 to afford 624-3 (4.2 g, 66%) as a white solid.

1H-NMR-624-3: (400 MHz, DMSO-d6) δ 9.91-9.69 (m, 2H), 8.00 (s, 1H), 7.54-7.49 (m, 1H), 7.44-7.38 (m, 1H), 7.35-7.26 (m, 2H), 2.73-2.66 (m, 2H), 2.31-2.20 (m, 2H), 1.15-1.07 (m, 3H), 1.02-0.95 (m, 3H)

### 4. Synthesis of 624-4

To a solution of 624-3 (2 g, 6.15 mmol, 1 equiv) in THF (32 mL) was added LAWESSON'S REAGENT (4.98 g, 12.3 mmol, 2 equiv). The mixture was stirred at 40° C. for 12 h under nitrogen atmosphere. The reaction mixture was concentrated in vacuum. The residue was dissolved in DCM (40 mL), washed with Sat. Na2CO3 (20 mL×2). The organic phase was dried over anhydrous Na2SO4, filtered and concentrated in vacuum. The residue was purified by flash silica gel chromatography eluted with EA/PE=1:9 to afford 624-4 (1.2 g, 60%) as a colorless oil.

1H-NMR-624-4: (400 MHz, METHANOL-d4) δ 9.29 (s, 1H), 7.56 (t, J=2.0 Hz, 1H), 7.44-7.38 (m, 2H), 7.32-7.27 (m, 1H), 2.99 (d, J=12.8 Hz, 2H), 2.79 (d, J=12.8 Hz, 2H), 1.15 (s, 3H), 1.09 (s, 3H)

### 5. Synthesis of 624-5

To a solution of 624-4 (1 g, 3.09 mmol, 1 equiv) and NH2Boc (435 mg, 3.71 mmol, 1.2 equiv) in dioxane (15 mL) was added Pd(OAc)2 (34.7 mg, 154 umol, 0.05 equiv), XPhos (147 mg, 309 umol, 0.1 equiv) and Cs2CO3 (1.41 g, 4.33 mmol, 1.4 equiv). The mixture was stirred at 95° C. for 12 h under nitrogen atmosphere. The reaction mixture was diluted with water (20 mL), extracted with EtOAc (20 mL×3). The combined organic layers were dried over anhydrous Na2SO4, filtered and concentrated in vacuum. The residue was purified by flash silica gel chromatography eluted with EA/PE=1:3 to afford 624-5 (1 g, 90%) as a yellow oil.

1H-NMR-624-5: (400 MHz, METHANOL-d4) δ 9.26 (s, 1H), 7.53 (s, 1H), 7.32-7.24 (m, 2H), 7.10-7.03 (m, 1H), 2.99 (d, J=12.4 Hz, 2H), 2.82 (d, J=12.4 Hz, 2H), 1.53 (s, 9H)

### 6. Synthesis of 624-6

To a solution of 624-5 (0.9 g, 2.5 mmol, 1 equiv) in DCM (8 mL) was added TFA (8 mL). The mixture was stirred at 20° C. for 1 hr. The reaction mixture was concentrated in vacuum. The residue was dissolved with EtOAc (20 mL), washed with Sat. NaHCO₃ (15 mL×2). The separated organic phase was dried over anhydrous Na2SO4, filtered and concentrated in vacuum to afford 624-6 (600 mg, crude) as a brown oil.

### 7. Synthesis of 624-7

To a solution of 624-6 (0.4 g, 1.54 mmol, 1 equiv) and I-2 (441 mg, 1.54 mmol, 1 equiv) in MeOH (8 mL) was added AcOH (264 uL, 4.63 mmol, 3 equiv). The mixture was stirred at 20° C. for 0-5 h under nitrogen atmosphere. Then NaBH3CN (193 mg, 3.08 n-mol, 2 equiv) was added to the mixture, the result mixture was stirred at 20° C. for 1.5 h under nitrogen atmosphere. The reaction mixture was diluted with Sat.NaHCO₃ (20 mL), extracted with EtOAc (15 mL×3). The combined organic layers were dried over anhydrous Na2SO4, filtered and concentrated in vacuum. The residue was purified by flash silica gel chromatography eluted with EA/PE=3:1 to afford 624-7 (0.7 g, 86%) as a yellow oil.

1H-NMR-624-7: (400 MHz, DMSO-d6) δ 9.29 (s, 1H), 8.60 (s, 1H), 7.94 (s, 1H), 6.96 (t, J=8.0 Hz, 1H), 6.54 (s, 1H), 6.47-6.39 (m, 2H), 6.21 (t, J=5.6 Hz, 1H), 4.38 (d, J=5.6 Hz, 2H), 3.46 (s, 2H), 2.73 (d, J=12.4 Hz, 2H), 2.62-2.57 (m, 1H), 2.51 (d, J=12.4 Hz, 2H), 1.59-1.45 (m, 5H), 1.41-1.33 (m, 1H), 0.98 (s, 3H), 0.89 (s, 3H), 0.79-0.69 (m, 5H)

### 8. Synthesis of 624

To a solution of 624-7 (0.2 g, 377 umol, 1 equiv) in DCM (1 ML) was added Py (183 uL, 2.27 mmol, 6 equiv) and bis(trichloromethyl) carbonate (56.0 mg, 189 umol, 0.5 equiv) at 0° C. The mixture was stirred at 20° C. for 0.5 h under nitrogen atmosphere. The reaction mixture was diluted with Sat.NaHCO₃ (20 ml), extracted with DCM (15 mL×3). The combined organic layers were dried over anhydrous Na2SO4, filtered and concentrated in vacuum. The residue was purified by flash silica gel chromatography eluted with EA/PE=3:1 give crude product. The crude product (300 mg) was purified by Prep-HPLC with the following conditions (Column: Phenomenex Luna C18 200*40 mm*10 um; Mobile Phase A: Water (0.2% formic acid), Mobile Phase B: ACN; Flow rate: 50 mL/min; Gradient: 15% B to 50% B in 8 min; Wave Length: 220 nm; RT1 (min): 8.0) to afford 624 (160 mg, 38%) as a yellow solid.

MS-624: (ES, m/z): [M+H]+ 556.2

1H NMR-624: (400 MHz, DMSO-d6) δ 9.45 (s, 1H), 8.17 (s, 1H), 7.88 (s, 1H), 7.75-7.69 (m, 1H), 7.64 (s, 1H), 7.51 (t, J=8.0 Hz, 1H), 7.43-7.33 (m, 2H), 7.00 (s, 1H), 3.24 (s, 2H), 2.98-2.91 (m, 2H), 2.81 (d, J=12.4 Hz, 2H), 2.77-2.68 (m, 2H), 1.95-1.83 (m, 1H), 1.69-1.53 (m, 4H), 1.50-1.38 (m, 1H), 1.12 (s, 3H), 1.06 (s, 3H), 0.90-0.77 (m, 4H).

### Example 620. Synthesis of Compound 625

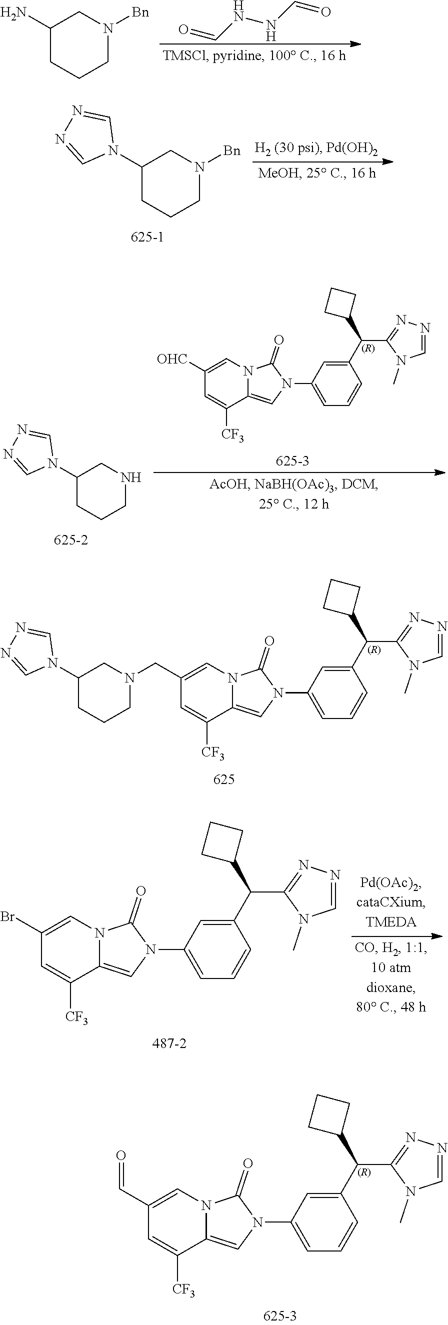

### 1. Synthesis of 625-1

To a solution of 1-benzylpiperidin-3-amine (1 g, 5.26 mmol, 1 eq) in pyridine (40 mL) was added Et3N (5.46 g, 53.97 mmol, 7.51 ml, 10.27 equiv), TMSCl (8.78 g, 80.83 mmol, 1026 mL, 15.38 equiv) and N-formamidoformamide (1.53 g, 17.34 mmol, 3.3 equiv). After addition, the resulting mixture was stirred at 100° C. for 16 h. The reaction mixture was partitioned between NaHCO₃10 mL and EtOAc 25 mL. The organic phase was separated, washed with brine (5 mL), dried over Na2SO4, filtered and concentrated under reduced pressure to give a residue. The residue was purified by column chromatography (SiO2, Dichloromethane: Methanol=10/1) to give 625-1 (710 mg 2.93 mmol, 55.75% yield) as a brown yellow liquid.

### 2. Synthesis of 625-2

To a solution of 625-1 (500 mg, 2.06 mmol, 1 equiv) in MeOH (20 MT) was added Pd(OH)2 (289.77 mg, 2.06 mmol, 1 equiv). The mixture was stirred at 25° C. for 16 hr under H2 atmosphere. The mixture was filtered and the filtrate concentrated under reduced pressure to give 625-2 (370 mg, crude) as a white solid.

### 3. Synthesis of 625-3

To a solution of 487-2 (2 g, 3.95 mmol, 1 equiv) in dioxane (70 mL) was added N,N,N′,N′-tetramethylethane-1,2-diamine (918 tug, 7.90 mmol, 2 equiv), Pd(OAc)2 (177 mg, 790 umol, 0.2 equiv), bis(1-adamantyl)-butyl-phosphane (283 mg 790 umol, 0.2 equiv) under N2. The suspension was degassed and purged with 112 and CO (2.5 MPa) for 3 times. The mixture was heated to 80° C. and stirred for 48 h under H2 and CO (2.5 MPa). The mixture was concentrated in vacuum to get a residue. Sat.Na2CO3 (300 mL) was added. The resulting solution was extracted with DCM:MeOH (10:1, 100 mL×3). The combined organic layers were washed with brine (100 mL), dried over Na2SO4, filtered and concentrated under reduced pressure to give a residue The residue was purified by column (SiO2) with petroleum ether/EtOAc=2:1 to 0:1 to DCM:MeOH=1:0 to 1:1 to get 625-3 (1 g, 40.5% yield) as a yellow solid.

1H-NMR-625: (400 MHz, CDCl3) δ 9.68 (s, 1H), 8.29 (s, 1H), 8.03 (s, 1H), 7.56-7.53 (m, 2H), 7.45-7.43 (m, 1H), 7.34-7.33 (m, 1H), 7.26-7.24 (m, 1H), 6.86 (s, 1H), 3.99-3.96 (m, 1H), 3.44 (s, 3H), 2.36-2.34 (m, 1H), 1.94-1.90 (m, 3H), 1.83-1.79 (m, 3H).

### 4. Synthesis of 625

To a solution of 625-2 (100 mg, 658 umol, 1 equiv) in DCM (10 mL) was added 625-3 (300 ng, 658 umol, 1 equiv), AcOH (39.56 mg, 658 umol, 1 equiv) at 25° C. and stirred for 1 h. NaBH(OAc)3 (279 mg, 1.32 mmol, 2 equiv) was added and stirred for 11 h. The mixture was poured into H2O (20 mL), extracted with DCM:MeOH (10:1, 20 mL×3). The combined organic layers were dried over Na2SO4, filtered and concentrated under reduced pressure to give a residue. The residue was purified by column (SiO₂) with petroleum ether/EtOAc=1:1 to 0:1 and then with DCM:MeOH=1: 0 to 1:1 to get a crude. The crude product was triturated with (DCM:MeOH): petroleum ether=(20:1, 2 mL): (20 m-L) to get a residue. The residue was purified by prep-HPLC (neutral) (column: Waters Xbridge Prep OBD C18 150*40 mm10 um; mobile phase: [water (NH4HCO3)-ACN]; B %: 30%-50%, 8 min Wave Length: 220 n; RT (min): 9.0 min) to get 625 (12 mg, 2.9%) as a yellow solid.

MS-625: (ES, m/z): [M+H]+ 592.3

1H-NMR-625: (400 MHz, DMSO) δ 8.64-8.62 (m, 2H), 8.32 (s, 1H), 7.71-7.67 (m, 3H), 7.45-7.41 (m, 1H), 7.29 (s, 1H), 7.20-7.18 (m, 1H), 7.03 (s, 1H), 4.38-4.36 (m, 1H), 4.26-4.24 (m, 1H), 3.42 (s, 2H), 3.40-3.36 (m, 3H), 3.20-3.18 (m, 1H), 2.96-2.94 (m, 1H), 2.70-2.67 (m, 1H), 2.45-2.43 (m, 1H), 2.25-2.20 (m, 1H), 2.08-2.06 (m, 1H), 1.98-1.96 (m, 1H), 1.78-1.67 (m, 7H), 1.57-1.53 (m, 1H).

### Example 621. Synthesis of Compound 626

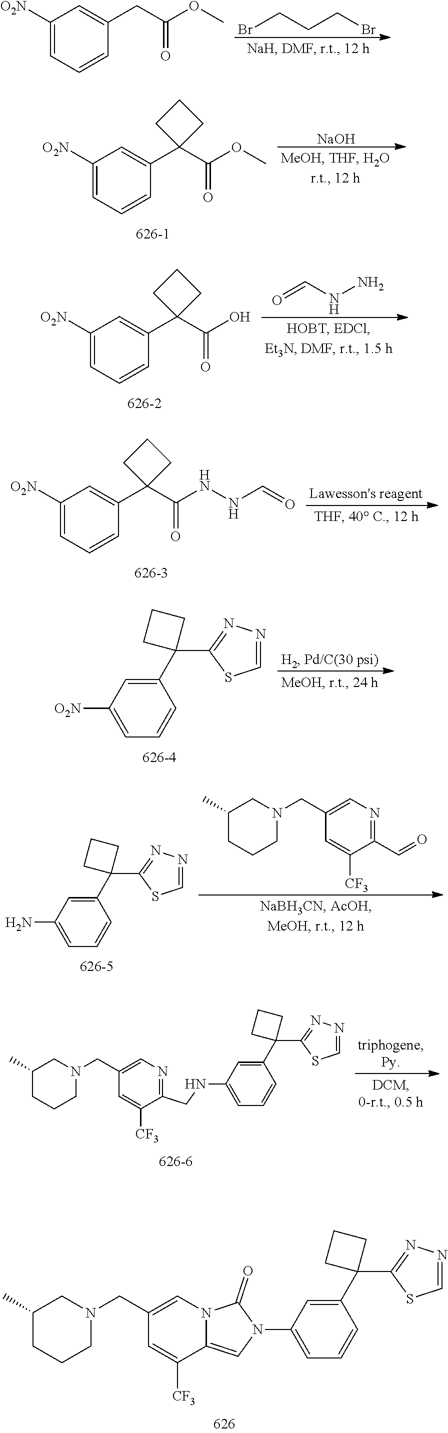

### 1. Synthesis of 626-1

To a solution of methyl 2-(3-nitrophenyl)acetate (10 g, 51.24 mmol, 1 equiv) and 1,3-dibromopropane (10.34 g, 51.24 mmol, 1 equiv) in DMF (360 mL) was added NaI (4.10 g, 102 mmol, 60% purity, 2 equiv) at 25° C. and stirred for 12 h. The mixture was poured into H2O (300 mL) at 0° C., extracted with EtOAc (200 mL×3). The combined organic layer was washed by brine (200 mL×3), dried by Na2SO4, filtered and concentrated in vacuum to get a residue. The residue was purified by column (SiO2) with petroleum ether/EtOAc=100:1 to 3:1 to get 626-1 (8.1 g, 33.6%) as a light yellow solid.

### 2. Synthesis of 626-2

To a solution of methyl 626-1 (8.5 g, 36.1 mmol, 1 equiv) in MeOH (80 mL), THF (20 mL) and 1H2O (20 mL) was added NaOH (2.89 g, 72.2 mmol, 2 equiv) at 25° C. and stirred for 12 h. The mixture was acidified to pH=3 with HCl (aq, 1M). The precipitate solids were collected by filtration and concentrated in vacuum to get 626-2 (7 g, 87.5%) as a white solid,

### 3. Synthesis of 626-3

To a solution of 626-2 (6.2 g, 28.0 mmol, 1 equiv) in DMF (80 mL) was added formohydrazide (2.52 g, 42.0 mmol, 1.5 equiv), HOBt (5.68 g, 42.0 mmol, 1.5 equiv), EDCI (8.06 g, 42.0 mmol, 1.5 equiv), TEA (851 g, 84.0 mmol, 3 equiv) at 25° C. The reaction mixture was stirred for 6 h at 25° C. The mixture was poured into H2O (300 mL), extracted with DCM:MeOH (20:1, 200 mL×3). The combined organic layers were dried over Na2SO4, filtered and concentrated under reduced pressure to give a residue. The residue was purified by column (SiO2) with PE/EA=1:0 to 0:1 to obtain 626-3 (6.5 g, 87.2%) as a light yellow oil.

1H-NMR-626-3: (400 MHz, DMSO), δ 9.85 (brs, 1H), 9.81 (brs, 1H), 8.21-8.20 (m, 1H), 8.13-8.11 (m, 1H), 7.99 (s, 1H), 7.82-7.80 (m, 1H), 7.67-7.65 (m, 1H), 2.83-2.80 (m, 2H), 2.49-2.46 (m, 2H), 1.90-1.81 (m, 2H).

### 4. Synthesis of 626-4

To a solution of 626-3 (3.3 g, 12.54 mmol, 1 equiv) in THF (80 mL) was added LAWESSON'S REAGENT (10.1 g, 25.0 mmol, 2 equiv). Then the mixture was heated to 40° C. and stirred for 12 h. The mixture was poured into sat. Na2CO3 (300 mL) at 0° C., extracted with EtOAc (200 mL×3). The combined organic layer was washed by brine (200 mL×3), dried by Na2SO4, filtered and concentrated in vacuum to get a residue. The residue was purified by column (SiO2) with petroleum ether/EtOAc=1:0 to 1:1 to get 626-4 (2.5 g, 63.3%) as a light yellow oil.

### 5. Synthesis of 626-5

To a solution of 626-4 (2.5 g, 9.57 mmol, 1 equiv) in EtOAc (100 mL) was added Pd/C (2 g, 10% purity) under H2. The suspension was degassed and purged with H2 for 3 times. The mixture was stirred under 112 (30 Psi) at 25° C. and stirred for 24 h. The mixture was filtered and washed with DCM/MeOH (1:1, 1 L). The filtrate was concentrated in vacuum to get a crude. The crude was purified by column (SiO2) with petroleum ether/EtOAc=101: to 1:1 to obtain 626-5 (1.5 g, 67.7%) as a light yellow oil. 1H-NMR-626-5 (400 MHz, CDCl3), δ 8.98 (s, 1H), 7.17-7.13 (m, 1H), 6.75-6.73 (m, 1H), 6.63-6.62 (m, 1H), 6.60-6.58 (m, 1H), 3.77 (brs, 2H), 3.00-2.97 (m, 2H), 2.85-2.82 (m, 2H), 2.30-2.27 (m, 1H), 2.01-1.99 (m, 1H).

### 6. Synthesis of 626-6

To a solution of 5-[[(3S)-3-methyl-1-piperidyl]methyl]-3-(trifluoromethyl)pyridine-2-carbaldehyde (198.03 ng, 691.69 umol, 1 equiv) in MeOH (5 mL) was added 626-5 (160 mg, 691 umol, 1 equiv), AcOH (41.54 mg, 691 umol, 1 equiv) at 25° C. and stirred for 1 h. NaBH3CN (86.93 mg, 1.38 mmol, 2 equiv) was added and stirred for 11 h. The mixture was poured into sat, NaHCO3 (100 mL) at 0° C., extracted with DCM (100 mL×3). The combined organic layer was washed by brine (200 mL×3), dried by Na2SO4, filtered and concentrated in vacuum to get a residue. The residue was purified by column (SiO2) with petroleum ether/EtOAc=1:0 to 0:1, then with DCM:MeOH=1:0 to 5:1 to obtain 626-6 (325 mg, 93.67%) as a yellow solid.

### 7. Synthesis of 626

To a solution of 626-6 (300 mg, 598 umol, 1 equiv) in DCM (10 mL) was added pyridine (283 mg, 3.59 mmol, 6 equiv), bis(trichloromethyl) carbonate (90 mg, 303 umol, 0.5 equiv) at 0° C. and stirred for 1 h. The same scale reaction was conducted with in parallel for 2 batches in total and work up with a small test scale (50 mg) together. The reaction mixture was diluted with Sat.NaHCO3 (50 mL), extracted with DCM (50 mL×3). The combined organic layers were dried over Na2SO4, filtered and concentrated in vacuum to get a residue. The residue was purified by column (SiO₂) with petroleum ether/EtOAc=5:1 to 0:1, then with DCM/MeOH=1:0 to 1:1 to get a crude. The crude was purified by prep-1-HPLC (neutral) (column: Waters Xbridge BEH C18 100*30 mm*10 um; mobile phase: [water (NH4HCO3)-ACN]; B %. 55%-85%, 8 min; Wave Length: 220 nm; RT1 (min): 5.2) to obtain 626 (145 mg) as a yellow solid,

1H-NMR-626: (400 MHz, DMSO) δ 9.48 (s, 1H), 7.83 (s, 1H), 7.75-7.73 (m, 1H), 7.64 (s, 1H), 7.51 (t, J=8 Hz, 1H), 7.41 (s, 1H), 7.33-7.31 (m, 1H), 6.99 (s, 1H), 3.23 (s, 2H), 2.97-2.83 (m, 4H), 2.76-2.71 (m, 2H), 2.17-2.12 (m, 1H), 1.99-1.85 (m, 2H), 1.65-1.57 (m, 4H), 1.48-1.39 (m, 1H), 0.89-0.81 (m, 4H).

### Example 622. Synthesis of Compound Synthesis of 627_P1&P2

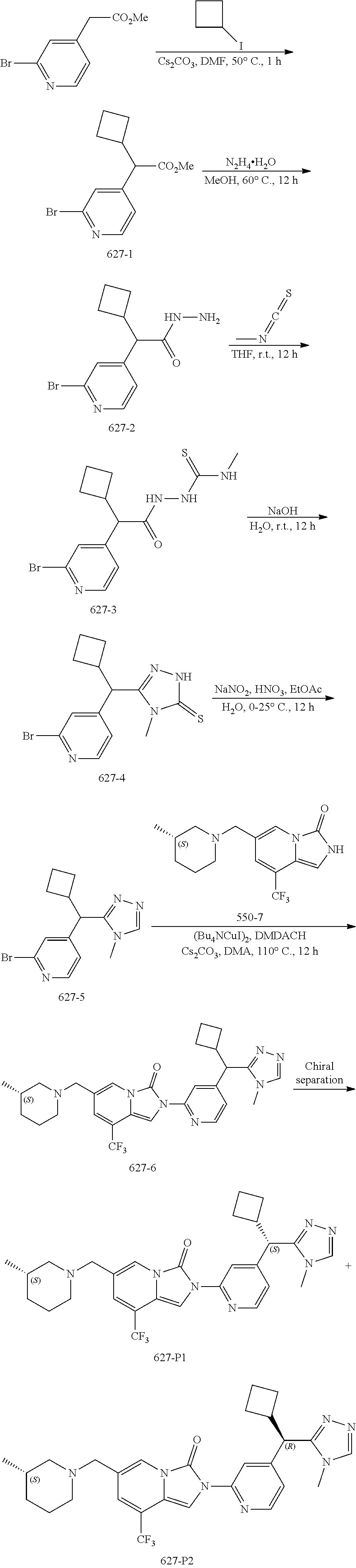

### 1. Synthesis of 627-1

To a solution of methyl 2-(2-bromo-4-pyridyl)acetate (2 g, 8.69 mmol, 1 equiv) in DMF (25 mL) was added iodocyclobutane (2.37 g, 13.04 mmol, 1.5 equiv), Cs2CO3 (5.66 g, 17.39 mmol, 2 equiv). The mixture was stirred for 1 h at 50′° C. under nitrogen atmosphere. The reaction mixture was diluted with H2O (50 ml) and extracted with EtOAc (100 ml×3). The combined organic layers were washed with brine (150 mL×3), dried over Na2SO4, filtered and concentrated under reduced pressure to give a residue. The residue was purified by column chromatography (SiO₂, Petroleum ether/Ethyl acetate=5:1 to 1:10) to afford 627-1 (2.1 g, 82.4%) as a brown solid.

1H-NMR-627-1: (400 MHz, CHLOROFORM-d) δ 8.30 (d, J=5.2 Hz, 1H), 7.43 (d, J=0.4 Hz, 1H), 7.19-7.18 (m, 1H), 3.69 (s, 3H), 3.51 (d, J=10.4 Hz, 1H), 2.98-2.85 (m, 1H), 2.26-2.15 (m, 1H), 1.93-1.81 (m, 4H), 1.60-1.56 (m, 1H)

### 2. Synthesis of 627-2

To a solution of 627-1 (3.96 g, 6.97 mmol, 1 equiv) in MeOH (20 mL) was added N2H4·H2O (7.05 g, 138.01 mmol, 98% purity, 19.81 equiv). The mixture was heated to 60° C. and stirred for 12 h at 60° C. The reaction mixture was concentrated under reduced pressure to give a residue. The residue was diluted with H2O (30 mL), and extracted with DCM (25 mL×3). The combined organic layers were dried over Na2SO4, filtered and concentrated under reduced pressure to afford 627-2 (4.1 g, crude) as a white solid.

### 3. Synthesis of 627-3

To a solution of 627-2 (3 g, 10.91 m mol, 1 equiv) in THF (35 mL) was added methylimino(thioxo)methane (1.75 g, 24.00 mmol, 2.2 equiv). The mixture was stirred for 12 h at 15° C. The mixture was concentrated under reduced pressure to remove THF and diluted with 1-20 (30 mL), and filtered. Then the mixture was filtered to get a filter cake and the filter cake was concentrated in vacuum to afford 627-3 (3.4 g, crude) as a white solid.

### 4. Synthesis of 627-4

To a solution of 627-3 (4.4 g, 12.32 mmol, 1 equiv) in H2O (40 mL) was added NaOH (3.94 g, 98.53 mmol, 8 equiv). The mixture was stirred for 12 h at 15° C. The mixture was adjusted to pH=2 by HC (2 M), and filtered. The filter cake was concentrated in vacuum to afford 627-4 (3 g, crude) as a white solid.

### 5. Synthesis of 627-5

HNO3 (5.63 g, 60.76 mmol, 68% purity, 10.31 equiv) was added to H2O (60 mL) to afford the diluted HNO3 solution (1M, 64.02 mL). To a solution of 627-4 (2 g, 5.90 mmol, 1 equiv) in H2O (20 mL) was added NaNO2 (4.07 g, 58.95 mmol, 10 equiv), EtOAc (4.16 g, 47.16 mmol, 8 equiv), diluted HNO3 solution (1 M, 64.02 mL) dropwise at 0° C. Then the mixture was stirred at 25° C. for 12 h. The solvent was diluted with H2O (30 mL) and adjusted pH to 12 by 2N NaOH, and extracted with DCM (150 mL×3). The combined organic layers were dried over Na2SO4 filtered and concentrated under reduced pressure to give a residue. The residue was purified by column chromatography (SiO₂, DCM/MeOH=50/1 to 5/1) to afford 627-5 (1.67 g, 78%) as a yellow oil.

1H-NMR-627-5: (400 MHz, CHLOROFORM-d) δ 8.29-8.28 (d, J=4 Hz, 1H), 8.08 (s, 1H), 7.37 (s, 1H), 7.14-7.13 (m, 1H), 3.85-3.83 (d, J=8 Hz, 1H), 3.45 (s, 3H), 3.35-3.21 (m, 1H), 2.31-2.27 (m, 1H), 1.93-1.66 (m, 5H)

### 6. Synthesis of 627-6-P1

To a solution of 627-5 (133.70 mg, 435.23 umol, 1 equiv) in dioxane (4 mL) was added 550-7 (150 mg, 478.75 umol, 1.1 equiv), Cs2CO3 (283.61 mg, 870.45 umol, 2 equiv), iodocopper;tetrabutylammonium;diiodide (48.73 mg, 43.52 umol, 0.1 equiv), N1,N2-dimethylcyclohexane-1,2-diamine (12.38 mg, 87.05 umol, 0.2 equiv) in glove box. The same scale reaction was conducted with in parallel for 2 batches in total and work up together. The reaction mixture was diluted with 1-20 (10 mL) and extracted with EA (15 mL×3). The combined organic layers were washed with brine (20 mL), dried over Na2SO4, filtered and concentrated under reduced pressure to give a residue. The residue was purified by column chromatography (SiO₂, Petroleum ether/Ethyl acetate=1/1 to DCM: MeOH=1:5) to get a product to afford 627-6 (400 mg, crude) as a yellow solid.

1H-NMR-627-6: (400 MHz, CHLOROFORM-d) δ 8.58 (s, 1H), 8.36-8.35 (d, J=4.0 Hz, 1H), 8.07 (br s, 1H), 7.64-7.49 (m, 2H), 7.05-7.04 (d, J=4.0 Hz, 1H), 6.96 (s, 1H), 4.05-4.02 (d, J=12.0 Hz, 1H), 3.47 (s, 3H), 3.43-3.32 (m, 1H), 3.21 (br s, 2H), 2.78-2.73 (m, 2H), 2.33-2.31 (m, 1H), 1.97-1.76 (m, 6H), 1.71-1.50 (m, 6H), 0.87-0.85 (d, J=8.0 Hz, 3H)

### 7. Synthesis of 627_P1&P2

The 627-6 (400 mg) was purified by Chiral separation with the following conditions (column: DAICEL CHIRALPAK AD (250 mm*30 mm, 10 um);mobile phase: [Neu-ETOH]; B %: 43%-43%, 8 min, Flow rate: 70 mL/min; Wave Length: 220/254 nm; RT1 (min): 1.18; RT2 (min): 1.40) to afford 627_P1 (118.1 mag) as a yellow solid and 627_P2 (157.9 mg) as a yellow solid,

MS-627_P1: (ES, m/z): [M+H]+ 540.3.

1H-NMR-627_P1: (400 MHz, DMSO-d6) δ 8.44-8.43 (d, J=4.0 Hz, 1H), 8.37-8.36 (d, J=4.0 Hz, 2H), 7.63 (s, 1H), 7.43 (s, 1H), 7.23-7.22 (d, J=4.0 Hz, 1H), 7.02 (s, 1H), 4.43-4.40 (d, J=12 Hz, 1H), 3.45 (s, 3H), 3.26-3.14 (m, 3H), 2.80-2.67 (m, 2H), 2.13-2.02 (m, 1H), 1.99-1.70 (m, 6H), 1.68-1.38 (m, 5H), 0.92-0.75 (m, 4H).

MS-627_P2: (ES, m/z): [M+H]+ 540.3.

1H-NMR-627_P2: (400 MHz, DMSO-d6) δ 8.44-8.43 (d, J=4.0 Hz, 1H), 8.37-8.36 (d, J=4.0 Hz, 2H), 7.63 (s, 1H), 7.43 (s, 1H), 7.23-7.22 (d, J=4.0 Hz, 1H), 7.02 (s, 1H), 4.43-4.40 (d, J=12 Hz, 1H), 3.45 (s, 3H), 3.26-3.14 (m, 3H), 2.80-2.67 (m, 2H), 2.13-2.02 (m, 1H), 1.99-1.70 (m, 6H), 1.68-1.38 (m, 5H), 0.92-0.75 (m, 4H).

### Example 623. Synthesis of Compound 628_P1&P2

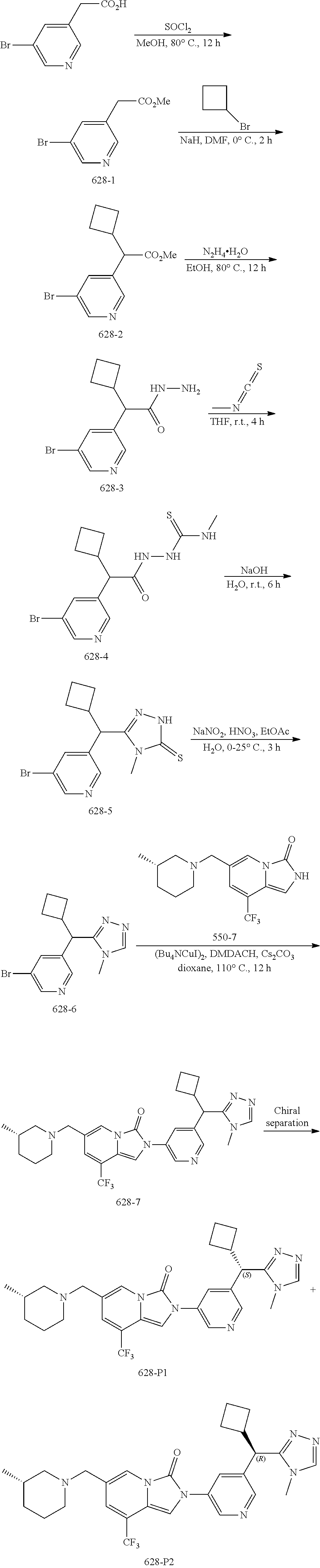

### 1. Synthesis of 628-1

To a solution of 2-(5-bromo-3-pyridyl)acetic acid (4 g, 18.52 mmol, 1 equiv) in MeOH (50 mL) was added SOCl2 (4 mL, 2.98 equiv). The mixture was stirred at 80° C. for 12 hr. The reaction mixture was concentrated under reduced pressure to give a residue. And then the residue was adjusted pH to 8 with saturated NaHCO₃ and extracted with EtOAc (20 mL×3). The combined organic layers were dried over anhydrous Na2SO4, filtered and concentrated under reduced pressure to give a residue. The residue was purified by silica gel column chromatography eluted with PE/EtOAc (4:1) to afford 628-1 (3.9 g, 92%) as a white solid.

1H-NMR-628-1: (400 MHz, CHLOROFORM-d) δ 8.53 (d, J=2.0 Hz, 1H), 8.37 (d, J=2.0 Hz, 1H), 7.75 (t, J=2.0 Hz, 1H), 3.66 (s, 3H), 3.56 (s, 2H)

### 2. Synthesis of 628-2

To a solution of NaH (363 mg, 9.08 mmol, 60% purity, 1.1 equiv) in DMF (20 mL) was added 628-1 (1.9 g, 8.26 mmol, 1 equiv). The mixture was cooled to 0° C. under nitrogen atmosphere. And then bromocyclobutane (19 ml, 41.3 mmol, 5 equiv) was added to reaction mixture at 0° C. The mixture was stirred at 0° C. for 2 hr. The reaction mixture was adjusted pH to 1 with 1 N HCl and extracted with EtOAc (20 mL×3). The combined organic layers were dried over anhydrous Na2SO4, filtered and concentrated under reduced pressure to give a residue. The residue was purified by silica gel column chromatography eluted with PE/EtOAc (9:1) to afford 628-2 (560 mg, 18%) as a colorless oil.

1H-NMR-628-2: (400 MHz, CHLOROFORM-d) δ 8.50 (d, J=2.0 Hz, 1H), 8.35 (d, J=2.0 Hz, 1H), 7.75 (t, J=2.0 Hz, 1H), 3.61 (s, 3H), 3.47 (d, J=10.8 Hz, 1H), 2.92-2.81 (m, 1H), 2.17-2.08 (m, 1H), 1.86-1.73 (m, 4H), 1.55-1.49 (m, 1H)

### 3. Synthesis of 628-3

To a solution of 628-2 (0.9 g, 3.17 mmol, 1 equiv) in EtOH (5 mL) was added N2H4·H2O (3.14 mL, 63.3 mmol, 98% purity, 20 equiv). The mixture was stirred at 80° C. for 12 hr. The reaction mixture was concentrated in vacuum to remove EtOH (5 mL). Then the mixture was diluted with water (10 mL), and extracted with DCM (10 mL×3). The combined organic layers were dried over anhydrous Na2SO4, filtered and concentrated under reduced pressure to afford 628-3 (800 mg, crude) as a white solid.

### 4. Synthesis of 628-4

To a solution of 628-3 (800 mg, 2.82 mmol, 1 equiv) in THF (8 mL) was added methylimino(thioxo)methane (411 mg, 5.63 mmol, 2 equiv). The mixture was stirred at 20° C. for 4 hr. The reaction mixture was diluted with water 20 mL and extracted with EtOAc (20 mL×3). The combined organic layers were dried over anhydrous Na2SO4, filtered and concentrated under reduced pressure to afford 628-4 (977 mg, crude) as a white solid.

### 5. Synthesis of 628-5

To a solution of NaOH (875.08 mg, 21.88 mmol, 8 equiv) in water (10 mL) was added 628-4 (977 mg, 2.73 mmol, 1 equiv). The mixture was stirred at 20° C. for 6 hr. The reaction mixture was filtered and the filter cake was concentrated under reduced pressure to afford 628-5 (800 mg, crude) as a white solid.

1H-NMR-628-5 (400 MHz, DMSO-d6) δ 13.73 (s, 1H), 8.72-8.43 (m, 2H), 7.90 (s, 1H), 4.34 (d, J=10.8 Hz, 1H), 3.27 (s, 3H), 3.07-2.95 (m, 1H), 2.09 (d, J=4.0 Hz, 1H), 1.84-1.68 (m, 5H)

### 6. Synthesis of 628-6

HNO3 (1.37 mL, 20.63 mmol, 68% purity, 10 equiv) was added to water (19 mL) to afford the diluted HNO3 solution (1 M, 20.37 mL). To a solution of 628-5 (700 mg, 2.06 mmol, 1 equiv) and NaNO2 (1.42 g, 20.6 mmol, 10 equiv) and EtOAc (1.26 g, 14.3 mmol, 1.4 mL) in water (7 mL) was added diluted HNO3 solution (1 M, 20.37 mL) dropwise at 0° C. Then the mixture was stirred at 25° C. for 3 hr. The reaction was quenched by the addition of saturated NaHCO₃ (30 mL). The organic layer was extracted with CH2Cl2 (30 ml×3) The organic phase was concentrated under reduced pressure to afford 628-6 (500 ng, crude) as a yellow solid,

1H-NMR-628-6 (400 MHz, DMSO-d6) δ 8.60 (d, J=2.4 Hz, 1H), 8.56 (d, J=2.0 Hz, 1H), 8.38 (s, 1H), 7.92 (t, J=2.0 Hz, 1H), 4.34 (d, J=10.8 Hz, 1H), 3.50 (s, 3H), 3.19-3.06 (m, 1H), 2.01 (s, 1H), 1.81-1.66 (m, 5H)

### 7. Synthesis of 628

To a solution of 628-6 (320 mg, 1.04 mmol, 1 equiv) and 550-7 (359 mg, 1.14 mmol, 1.1 equiv) in dioxane (3 mL) was added Cs2CO3 (679 mg, 2.08 mmol, 2 equiv) and N1,N2-dimethylcyclohexane-1,2-diamine (29.6 mg, 208 umol, 0.2 equiv), iodocopper;tetrabutylammonium; diiodide (117 mg, 104 umol, 0.1 equiv). The mixture was stirred at 110° C. for 12 hr. The reaction mixture was diluted with water 30 mL and extracted with EtOAc (30 mL×3). The combined organic layers were dried over anhydrous Na2SO4, filtered and concentrated under reduced pressure to give a residue. The residue was purified by flash silica gel chromatography elated with DCM/MeOH (10:1) to afford 628-7 (430 mg, 76%) as a yellow solid.

### 8. Synthesis of 628_P1&P2

The 628-7 (430 mg) was purified by Chiral separation with the following conditions (column: DAICEL CHIRALCEL OD (250 mm*30 nm, 10 um); Mobile Phase: [Neu-ETOH]; B %: 40%-40%, 11 min, Flow rate: 70 mL/min; Wave Length: 220/254 nm; RT1 (min): 3.23; RT2 (min): 6.59) to afford crude product 1 and 628_P2 (92.2 mg, 21%) as a yellow solid. The crude product 1 (120 mg) was further purified by Prep-HPLC with the following conditions (Column: Waters Xbridge Prep OBD C18 150*40 nm*10 um; Mobile Phase A: Water (10 mmol/L NH4HCO3), Mobile Phase B: ACN; Flow rate: 50 mL/min; Gradient: 35% B to 65% B in 8 min; Wave Length: 220 nm; RT1 (min): 800) to afford 628 P1 (61.4 mg, 51%) as a yellow solid.

MS-628_P1: (ES, m/z): [M+H]+ 540.3.

1H-NMR-628_P1: (400 MHz, DMSO-d6) δ 8.94 (d, J=2.4 Hz, 1H), 8.50 (d, J=2.0 Hz, 1H), 8.36 (s, 1H), 8.15 (t, J=2.0 Hz, 1H), 7.65 (s, 1H), 7.48 (s, 1H), 7.02 (s, 1H), 4.38 (d, J=10.4 Hz, 1H), 3.49 (s, 3H), 3.25 (s, 2H), 2.81-2.70 (m, 2H), 2.14-2.03 (m, 1H), 1.94-1.88 (m, 1H), 1.87-1.68 (m, 6H), 1.66-1.51 (m, 4H), 1.51-1.41 (m, 1H), 0.89-0.79 (m, 4H)

MS-628_P2: (ES, m/z): [M+H]+ 540.3.

1H-NMR-628_P2: (400 MHz, DMSO-d6) δ=8.94 (d, J=2.4 Hz, 1H), 8.50 (d, J=1.6 Hz, 1H), 8.36 (s, 1H), 8.15 (t, J=2.0 Hz, 1H), 7.64 (s, 1H), 7.47 (s, 1H), 7.02 (s, 1H), 4.38 (d, J=10.4 Hz, 1H), 3.49 (s, 3H), 3.25 (s, 2H), 2.80-2.70 (m, 2H), 2.14-2.02 (m, 1H), 1.94-1.88 (m, 1H), 1.88-1.74 (m, 5H), 1.74-1.52 (m, 5H), 1.48-1.42 (m, 1H), 0.90-0.78 (m, 4H).

### Example 624. Synthesis of Compound 629

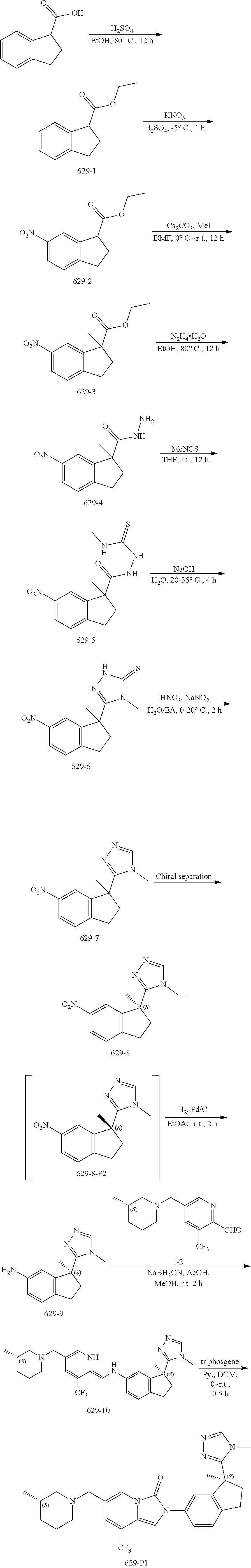

### 1. Synthesis of 629-1

To a solution of indane-carboxylic acid (4 g, 24.6 mmol, 1 equiv) in EtOH (40 mL) was added H₂SO₄ (1.31 mL, 24.6 mmol, 1 equiv). The mixture was stirred at 80° C. for 12 b. The reaction mixture was concentrated under reduced pressure to remove solvent. The residue was purified by flash silica gel chromatography eluted with PE/EA=10:1 to afford 629-1 (4 g, 85%) as a colorless oil

### 2. Synthesis of 629-2

To a solution of 629-1 (15.0 g, 78.8 mmol, 1.0 equiv) in H₂SO₄ (100 mL) was added KNO₃ (7.9 g, 78.8 mmol, 1.0 equiv) in 112SO₄ (50 mL) dropwise at −5° C. The mixture was stirred at −5° C. for 1 hr. The reaction was slowly poured into sat Na₂SO₃ (300 mL) at 0° C., and the resulting mixture was extracted with EtOAc (100 mL×2). The organic phase was washed with brine (30 mL), dried over anhydrous Na2SO4, concentrated in vacuum to give a residue. The residue was purified by flash silica gel chromatography eluted with PE/EA=5:1 to afford 629-2 (5.2 g, 28% yield) as colorless oil.

### 3. Synthesis of 629-3

To a solution of 629-2 (5.2 g, 22.1 mmol, 1 equiv), Cs2CO₃ (36.0 g, 110 mmol, S equiv) in DMF (60 mL) was added MeI (4.13 mL, 66.32 mmol, 3 equiv) at 0° C. The mixture was stirred at 25° C. for 12 hr. The reaction was poured into water (40 mil) and the resulting mixture was extracted with EtOAc (100 mL×3). The organic phase was washed with brine (50 mL), dried over anhydrous Na₂SO₄, concentrated in vacuum to give a residue. The residue was purified by flash silica gel chromatography eluted with PE/EA=4:1 to afford 629-3 (374 g, 68%) as yellow oil.

### 4. Synthesis of 629-4

To a solution of 629-3 (1.3 g, 5.22 mmol, 1 equiv) in EtOH (15 mL) was added N₂H₄·H₂O (7.76 mL, 156.46 umol, 98% purity, 30 equiv). The mixture was stirred at 80° C. for 12 h. The reaction was poured into water (10 mL) and the resulting mixture was extracted with EtOAc (10 mL×2). The organic phase was washed with brine (10 mL), dried over anhydrous Na₂SO₄, concentrated in vacuum to afford 629-4 (1.08 g, 88%) as a white solid.

¹H-NMR-629-4: (400 MHz, DMSO-d6) δ 8.98-9.15 (m, 1H) 8.15-8.25 (m, 1H) 8.08 (d, J=8.4, 1H) 7.48 (d, J=8.4 Hz, 1H) 4.12-4.32 (m, 2H) 2.90-3.11 (m, 2H) 2.58-2.73 (m, 1H) 1.94-2.00 (m, 1H) 1.42-1.55 (m, 3H).

### 5. Synthesis of 629-5

To a solution of 629-4 (11.08 g, 4.59 mmol, 1 equiv) in THF (15 ml) was added methylimino(thioxo)methane (627 uL, 9.18 mmol, 2 equiv). The mixture was stirred at 20° C. for 12 hr. The reaction was poured to water (50 mL) to form solid. The solid was filtered and the filter cake was concentrated to afford 629-5 (132 g, 93%) as a yellow solid,

¹H-NMR-629-5: (400 MHz, DMSO-d6) δ 9.59 (s, 1H), 9.12-9.22 (m, 1H), 8.25-8.34 (m, 1H), 8.06 (d, J=2.0 Hz, 1H), 7.65-7.75 (m, 1H), 7.45-7.55 (m, 1H), 3.00-3.07 (m, 2H), 2.82-2.95 (m, 3H), 2.66-2.75 (m, 1H), 1.98-2.08 (m, 1H), 1.51-1.60 (m, 3H).

### 6. Synthesis of 629-6

To a solution of NaOH (1.04 g, 25.9 mmol, 8 equiv) in H2O (10 mL) was added 629-5 (1.32 g, 4.28 mmol, 1 equiv), the mixture was stirred at 20° C. for 2 h. Then the mixture was stirred at 35° C. for 2 h. The combined mixture was diluted with water (15 mL), adjusted pH to 3 by 1N HCl. Then the mixture was filtered and the filter cake was concentrated in vacuum to afford 629-6 (950 mg, 77%) as a yellow solid.

¹H-NMR-629-6: (400 MHz, METHANOL-d₄) δ 8.21-8.17 (m, 1H), 7.91 (d, J=2.0 Hz, 1H), 7.56 (d, J=8.4 Hz, 1H), 3.29-3.22 (m, 2H), 3.21 (s, 3H), 2.73-2.61 (m, 1H), 2.40-2.30 (m, 1H), 1.74 (s, 3H)

### 7. Synthesis of 629-7

HNO₃ (1.71 mL, 25.8 mmol, 68% purity, 10 equiv) was added to H₂O (23.9 mL) to afford the diluted HNO₃ solution (1M, 25.6 mL). To a solution of 629-6 (0.75 g, 2.58 mmol, 1 equiv) and NaNO₂ (1.78 g, 25.8 mmol, 10 equiv) in H2O (7.5 mL) and EtOAC (750 uL, 7.66 mmol, 3.0 equiv) was added diluted HNO₃ solution (1 M, 25.6 mL) dropwise at 0° C., Then the mixture was stirred at 20° C. for 2 h. The reaction mixture was quenched by the addition of NaHCO₃ (aq.) (90 mL). The aqueous layer was extracted with DCM (45 mL×3). The aqueous phase was concentrated under reduced pressure to afford 629-7 (620 mg, crude) as a yellow solid.

### 8. Synthesis of 629-8

The 629-7 (620 mg) was purified by Chiral separation with the following conditions (column: Chiralpak 1H, (250 mm*30 mm, 10 um);mobile phase: [Neu-ETOH]; B %: 44%-44%, 9 min, Flow rate: 72 mL/min; Wave Length: 220/254 nm; RT1 (min): 3.72) to afford 629-8 (230 mg, 33%) as a yellow solid.

¹H-NMR-629-8: (400 MHz, METHANOL-d₄) δ 8.39 (s, 1H), 8.22-8.15 (m, 1H), 7.83 (d, J=1.6 Hz, 1H), 7.56 (d, J=8.4 Hz, 1H), 3.33 (s, 3H), 3.29-3.18 (m, 2H), 2.73-2.62 (m, 1H), 2.44-2.32 (m, 1H), 1.82 (s, 3H).

### 9. Synthesis of 629-9

To a solution of 629-8 (230 mg 890 umol, 1 equiv) in EtOAc (5 mL) was added Pd/C (0.2 g, 10% purity), the mixture was stirred at 20° C. for 2 h under H₂ (15 psi). The reaction mixture was filtered through celite, the filtrate was concentrated in vacuum to afford 629-9 (180 mg, crude) as a white solid.

¹H-NMR-629-9: (400 MHz, DMSO-d₆) δ 8.39 (s, 1H), 7.00 (d, J=8.0 Hz, 1H), 6.65-6.46 (m, 1H), 6.10 (d, J=2.0 Hz, 1H), 4.95 (s, 2H), 3.18 (s, 3H), 3.06-2.88 (m, 2H), 2.49-2.39 (m, 1H), 2.16-2.06 (m, 1H), 1.67 (s, 3H)

### 10. Synthesis of 629-10

To a solution of 629-9 (0.18 g, 788 umol, 1 equiv) and 1-2 (226 mg, 788 umol, 1 equiv) in MeOH (4 mL) was added AcOH (135 uL, 2.37 mmol, 3 equiv), the mixture was stirred at 20° C. for 1 h, then NaBH₃CN (99.1 mg, 1.58 mmol, 2 equiv) was added to the mixture, the result mixture was stirred at 20° C. for 1 h under nitrogen atmosphere. The reaction mixture was diluted with Sat.NaHCO₃ (12 mL), extracted with EtOAc (10 mL×3). The combined organic layers were dried over anhydrous Na₂SO₄, filtered and concentrated in vacuum. The residue was purified by silica gel column chromatography eluted with CH2Cl2/MeOH (10:1) to afford 629-10 (240 mg, 61%) as a white solid.

¹H-NMR-629-10: (400 MHz, METHANOL-d₄) δ 8.65 (s, 1H), 8.32 (s, 1H), 8.09 (s, 1H), 7.09 (d, J=8.0 Hz, 1H), 6.70-6.61 (m, 1H), 6.17 (d, J=1.6 Hz, 1H), 4.49 (s, 2H), 3.66-3.56 (m, 2H), 3.37 (s, 2H), 3.11 (s, 3H), 3.05-2.95 (m, 1H), 2.85-2.75 (m, 2H), 2.52-2.41 (m, 1H), 2.23-2.14 (m, 1H), 2.03-1.95 (m, 1H), 1.76-1.67 (m, 6H), 1.64-1.52 (m, 1H), 0.98-0.85 (m, 4H)

### 11. Synthesis of 629_P1

To a solution of 629-10 (210 mg, 421 umol, 1 equiv) in DCM (2.5 mL) was added Py (204 uL, 2.53 mmol, 6 equiv) and bis(trichloromethyl) carbonate (62.5 mg, 210 umol, 0.5 eq) at 0° C. Then the mixture was stirred at 20° C. for 0.5 h under nitrogen atmosphere. The reaction mixture was diluted with Sat.NaHCO₃ (15 mL), extracted with DCM (10 mL×3). The combined organic layers were dried over anhydrous Na₂SO₄, filtered and concentrated in vacuum. The residue was purified by flash silica gel chromatography eluted with CH₂Cl₂/MeOH=10:1 to afford crude product. The crude product (180 mg) was purified by Prep-HPLC with the following conditions (column: Phenomenex Luna C18 200*40 mm*10 um; mobile phase A: Water (0.2% formic acid), Mobile Phase B: ACN; Flow rate: 50 mL/min; Gradient: 10% B to 45% B in 8.0 min; Wave Length: 220 nm; RT1 (min): 7.5) to afford 629 P1 (86 mg, 39%) as a yellow solid.

MS-629_P1: (ES, m/z): [M+H]⁺ 525.3.

¹H-NMR-629_P1: (400 MHz, CHLOROFORM-d) δ 8.28 (s, 1H), 8.09 (s, 1H), 7.66 (s, 1H), 7.59-7.55 (m, 1H), 7.44 (d, J=8.0 Hz, 1H), 7.27 (d, J=2.0 Hz, 1H), 7.17 (s, 1H), 6.75 (s, 1H), 3.55 (d, J=8.0 Hz, 2H), 3.26 (s, 3H), 3.23-3.14 (m, 311), 3.09-3.04 (m, 1H), 2.59-2.49 (m, 1H), 2.41-2.33 (m, 1H), 2.23-2.13 (m, 1H), 1.90 (s, 4H), 1.88-1.72 (m, 4H), 1.02-0.95 (m, 1H), 0.92 (d, J=6.4 Hz, 3H).

### Example 625. IC50 Screening

### Experiment Procedure

a) Thaw UBE1, UBCH5b, CBL-B, Biotin-Ubiquitin, CBL-B assay buffer, and ATP on ice. Aliquot each protein, CBL-B assay buffer, and ATP into single-use aliquots and stored at −80° C. immediately.
b) Transfer 100 mL test compound (10 mM stock solution, start at 100 uM, 1:3 dilution, 11 points) and then backfill 100 mL DMSO to each well designated for the “Test compound” by Echo.
c) Transfer 200 mL DMSO to each well designated for the “High Control”, “Low Control” by Echo.
d) Carefully calculate the amount of proteins needed. Prepare appropriate amounts of diluted proteins; dilute only the amount required for the assay. Do not store diluted proteins. Keep the diluted reagents on ice until use.
e) Add 2.5 μl diluted CBL-B (40 nM) for the “High Control” and “Test compound”, the final concentration is 10 nM. For the “Low Control”, add 2.5 μL buffer each well. Pre-incubate for 15 min.
f) Centrifuge at 1000 rpm for 1 minute and shake plate at a speed of 600 rpm for 5 minutes.
g) Prepare the Master Mixture (E1E2) using diluted reagents: N wells×(1 μl Biotin-Ub+0.5 μl diluted UBE1+1 μl diluted UBCH5+5 μl ATP). Pre-incubation for 10 min.
h) Add master mixture to each well. Centrifuge at 1000 rpm for 1 minute and shake plate at a speed of 600 rpm for 5 minutes.
i) Incubate the reaction at 30° C. for 20 minutes. Cover the plate with a plate sealer.
j) Dilute Mab Anti GST-Tb cryptate donor (1:100) and Streptavidin-d2 acceptor (1:100) using PPI-Terbium detection buffer. Prepare only the amount required for the assay Add 5 μL diluted donor and 5 μL acceptor mixture into each well. Centrifuge at 1000 rpm for 1 minute and shake plate at a speed of 600 rpm for 5 minutes. Incubate at room temperature for one hour.
k) Read the fluorescent intensity with EnVison2104 (PerkinElmer Life Sciences). Blank value is subtracted from all other values. Tb-donor emission should be measured at 615 nM followed by dye-acceptor emission at 665 nM.

### Plate Map for IC50 Screen (Dose Titration of 11 Doses for 2 Reps)

a) Dilute cpds in 384 well dilution plate according to the dilution plate map
b) Cpds starting conc: 10 mM
c) Final cpd conc in assay: 100000, 33333.3, 11111.1, 3703.7, 1234.57, 411.52, 137.17, 45.72, 15.24, 5.08, 1.69 and 0 nM.
d) High control wells (HC): DMSO vehicle, with Cbl-b
e) Low control wells (LC): DMSO vehicle, No Cbl-b

### Data Analysis

### IC50 Screening Data Analysis

a) Calculate average of HC and LC samples
b) Calculate plate Z′ Factor:

$${Z}^{′} = 1 - 3 \times \frac{S T {D}_{HC} + S T {D}_{LC}}{A V {G}_{HC} - A V {G}_{LC}}$$

The Z′ should be >0.5. Otherwise, consider the plate failed QC and repeat the experiment.
c) Calculate percent vehicle of each test well:

$$\% Vehicle = 100 \times \frac{Test cpd - {Ave}_{L C}}{{Ave}_{H C} - {Ave}_{L C}}$$

d) Fit the cpd IC50 from non-linear regression equation (Equation: 201, XLfit5.3.1.3):

Y=Bottom+(Top−Bottom)/(1+10\hat{ }((Log IC50−X)*HillSlope))
X: Log of cpd concentration
Y: Percent inhibition (% inh)
   Top and Bottom: Plateaus in same units as Y
log IC50: same log units as X
HillSlope: Slope factor or Hill slope
   Compound IC₅₀s were binned into A through F as follows: A indicates <100 nM, B indicates 100 nM-300 nM, C indicates 301 nM-1,000 nM, D indicates 1,001 nM-3,000 nM, E indicates 3,001 nM-10,000 nM, and F indicates >10,000 nM.

TABLE 2

| Cmpd No. | CBL-B TR-FRET activity IC₅₀ |
|---|---|
| 1 | C |
| 2 | C |
| 3 | E |
| 4 | F |
| 5 | F |
| 6 | F |
| 7 | B |
| 8 | B |
| 9 | B |
| 10 | A |
| 11 | A |
| 12 | A |
| 13 | A |
| 14 | A |
| 15 | B |
| 16 | F |
| 17 | F |
| 18 | B |
| 19 | D |
| 20 | E |
| 21 | A |
| 22 | A |
| 23 | A |
| 24 | A |
| 25 | A |
| 26 | B |
| 27 | F |
| 28 | B |
| 29 | A |
| 30 | D |
| 31 | C |
| 32 | A |
| 33 | A |
| 34 | A |
| 35 | E |
| 36 | C |
| 37 | A |
| 38 | C |
| 39 | A |
| 40 | A |
| 41 | A |
| 42 | A |
| 43 | E |
| 44 | A |
| 45 | A |
| 46 | D |
| 47 | F |
| 48 | A |
| 49 | A |
| 50 | A |
| 51 | C |
| 52 | E |
| 53 | A |
| 54 | B |
| 55 | A |
| 56 | A |
| 57 | A |
| 58 | D |
| 59 | A |
| 60 | A |
| 61 | B |
| 62 | A |
| 63 | B |
| 64 | D |
| 65 | C |
| 66 | A |
| 67 | A |
| 68 | A |
| 69 | A |
| 70 | C |
| 71 | B |
| 72 | C |
| 73 | E |
| 74 | B |
| 75 | B |
| 76 | B |
| 77 | A |
| 78 | A |
| 79 | A |
| 80 | C |
| 81 | C |
| 82 | A |
| 83 | F |
| 84 | F |
| 85 | A |
| 86 | C |
| 87 | B |
| 88 | D |
| 89 | B |
| 90 | B |
| 91 | A |
| 92 | C |
| 93 | B |
| 94 | E |
| 95 | C |
| 96 | A |
| 97 | A |
| 98 | B |
| 99 | A |
| 100 | A |
| 101 | D |
| 102 | A |
| 103 | B |
| 104 | B |
| 105 | B |
| 106 | B |
| 107 | A |
| 108 | A |
| 109 | A |
| 110 | A |
| 111 | A |
| 112 | A |
| 113 | C |
| 114 | A |
| 115 | A |
| 116 | A |
| 117 | A |
| 118 | A |
| 119 | A |
| 120 | A |
| 121 | A |
| 122 | C |
| 123 | A |
| 124 | A |
| 125 | A |
| 126 | A |
| 127 | C |
| 128 | A |
| 129 | B |
| 130 | B |
| 131 | A |
| 132 | F |
| 133 | F |
| 134 | C |
| 135 | A |
| 136 | C |
| 137 | A |
| 138 | D |
| 139 | B |
| 140 | A |
| 141 | D |
| 142 | F |
| 143 | F |
| 144 | F |
| 145 | B |
| 146 | F |
| 147 | F |
| 148 | F |
| 149 | F |
| 150 | A |
| 151 | C |
| 152 | F |
| 153 | A |
| 154 | A |
| 155 | E |
| 156 | A |
| 157 | A |
| 158 | E |
| 159 | A |
| 160 | D |
| 161 | A |
| 162 | D |
| 163 | A |
| 164 | E |
| 165 | A |
| 166 | A |
| 167 | A |
| 168 | B |
| 169 | E |
| 170 | D |
| 171 | A |
| 172 | A |
| 173 | E |
| 174 | A |
| 175 | D |
| 176 | A |
| 177 | A |
| 178 | C |
| 179 | B |
| 180 | E |
| 181 | F |
| 182 | C |
| 183 | A |
| 184 | A |
| 185 | B |
| 186 | B |
| 187 | B |
| 188 | F |
| 189 | A |
| 190 | A |
| 191 | C |
| 192 | A |
| 193 | A |
| 194 | A |
| 195 | B |
| 196 | C |
| 197 | E |
| 198 | A |
| 199 | A |
| 200 | F |
| 201 | A |
| 202 | A |
| 203 | C |
| 204 | A |
| 205 | A |
| 206 | C |
| 207 | A |
| 208 | F |
| 209 | A |
| 210 | F |
| 211 | D |
| 212 | B |
| 213 | A |
| 214 | A |
| 215 | A |
| 216 | A |
| 217 | A |
| 218 | D |
| 219 | A |
| 220 | E |
| 221 | B |
| 222 | A |
| 223 | A |
| 224 | F |
| 225 | A |
| 226 | A |
| 227 | A |
| 228 | A |
| 229 | A |
| 230 | F |
| 231 | E |
| 232 | C |
| 233 | A |
| 234 | A |
| 235 | D |
| 236 | A |
| 237 | A |
| 238 | D |
| 239 | A |
| 240 | B |
| 241 | C |
| 242 | F |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | D |
| 255 | A |
| 256 | F |
| 257 | F |
| 258 | A |
| 259 | A |
| 260 | A |
| 261 | A |
| 262 | A |
| 263 | A |
| 264 | A |
| 265 | A |
| 266 | A |
| 267 | F |
| 268 | F |
| 269 | A |
| 270 | F |
| 271 | A |
| 272 | A |
| 273 | A |
| 274 | A |
| 275 | A |
| 276 | A |
| 277 | F |
| 278_p1 | A |
| 278_p2 | A |
| 279 | C |
| 280 | F |
| 281 | F |
| 282 | F |
| 283 | F |
| 284 | C |
| 285 | B |
| 286 | A |
| 287_P1 | A |
| 287_P2 | A |
| 288 | A |
| 289 | F |
| 290 | A |
| 291 | F |
| 292 | B |
| 293 | B |
| 294 | D |
| 295 | A |
| 296 | A |
| 297 | A |
| 298 | A |
| 299 | A |
| 300 | D |
| 301 | B |
| 302 | C |
| 303 | F |
| 304 | A |
| 305 | F |
| 306 | F |
| 307 | A |
| 308 | A |
| 309 | B |
| 310 | A |
| 311 | F |
| 312 | A |
| 313 | A |
| 314 | F |
| 315 | A |
| 316 | A |
| 317 | A |
| 318 | A |
| 319 | A |
| 329 | A |
| 321 | A |
| 322 | A |
| 323 | A |
| 324 | A |
| 325 | A |
| 326 | A |
| 327 | A |
| 328 | D |
| 329 | D |
| 330 | C |
| 331 | C |
| 332_p1 | F |
| 332-p2 | A |
| 333 | B |
| 334 | A |
| 335 | F |
| 336 | F |
| 337 | A |
| 338 | A |
| 339_P1 | A |
| 339_P2 | D |
| 340_P1 | A |
| 340_P2 | D |
| 341 | A |
| 342 | F |
| 343 | B |
| 344 | F |
| 345 | A |
| 346 | F |
| 347 | A |
| 348 | B |
| 349 | A |
| 350 | B |
| 351_P1 | D |
| 351_P2 | A |
| 352_P1 | F |
| 352_P2 | B |
| 353 | F |
| 354 | C |
| 355 | F |
| 356 | A |
| 357 | A |
| 358 | F |
| 359 | F |
| 360 | F |
| 361 | F |
| 362_P1 | A |
| 362_P2 | B |
| 363_P1 | F |
| 363_P2 | A |
| 364_P1 | C |
| 364_P2 | A |
| 365 | D |
| 366 | F |
| 367 | E |
| 368 | A |
| 369 | F |
| 370 | F |
| 371 | F |
| 372 | F |
| 373 | B |
| 374 | C |
| 375 | B |
| 376 | A |
| 377 | A |
| 378 | A |
| 379 | A |
| 380_P1 | D |
| 380_P2 | A |
| 381_P1 | D |
| 381_P2 | A |
| 382_P1 | A |
| 382_P2 | D |
| Patent Cmpd No | Updated Activity |
| 383 | F |
| 384 | F |
| 385 | F |
| 386 | F |
| 387 | F |
| 388 | F |
| 389 | F |
| 390 | F |
| 391 | F |
| 392 | F |
| 393 | A |
| 394 | B |
| 395 | B |
| 396 | B |
| 397 | B |
| 398 | A |
| 399 | A |
| 400 | F |
| 401_P1 | A |
| 401_P2 | F |
| 402 | F |
| 403_P1 | F |
| 403_P2 | C |
| 404 | F |
| 403 | F |
| 404 | F |
| 405 | F |
| 406 | B |
| 407 | F |
| 408 | A |
| 409 | A |
| 410 | F |
| 411 | F |
| 412 | A |
| 413 | A |
| 414 | A |
| 415 | A |
| 416 | A |
| 417 | A |
| 418 | E |
| 419 | F |
| 420 | F |
| 421 | A |
| 422 | A |
| 423 | F |
| 424 | F |
| 425 | F |
| 426 | F |
| 427 | F |
| 428 | F |
| 429 | B |
| 430 | C |
| 431 | F |
| 432 | F |
| 433 | A |
| 434_P1 | F |
| 434_P2 | D |
| 435 | F |
| 436 | F |
| 437 | A |
| 438 | A |
| 439 | B |
| 440 | F |
| 441 | F |
| 442 | F |
| 443 | F |
| 44/ | F |
| 445 | F |
| 446 | F |
| 447 | F |
| 448 | F |
| 449 | A |
| 450 | A |
| 451 | A |
| 452 | F |
| 453 | C |
| 454 | D |
| 455 | F |
| 456 | F |
| 457 | F |
| 458 | E |
| 459 | F |
| 460 | F |
| 461 | F |
| 462 | F |
| 463 | F |
| 464 | A |
| 465 | A |
| 466 | A |
| 467 | A |
| 468 | A |
| 469 | A |
| 470_P1 | F |
| 470_P2 | F |
| 471 | A |
| 472_P1 | B |
| 472_P2 | A |
| 473 | A |
| 474 | A |
| 475 | E |
| 476 | C |
| 477 | A |
| 478 | F |
| 479 | E |
| 480 | A |
| 481 | A |
| 482 | A |
| 483 | A |
| 484 | A |
| 485 | A |
| 486 | A |
| 487 | A |
| 488 | A |
| 489 | A |
| 490 | A |
| 491 | A |
| 492 | F |
| 493 | F |
| 494 | A |
| 495 | B |
| 496 | F |
| 497 | A |
| 498 | A |
| 499 | A |
| 500 | A |
| 501 | A |
| 502 | A |
| 503 | A |
| 504 | A |
| 505 | A |
| 506_P1 | A |
| 506_P2 | A |
| 507 | A |
| 508 | A |
| 509 | A |
| 510 | C |
| 511 | F |
| 512 | F |
| 513 | F |
| 514 | A |
| 515 | F |
| 516 | F |
| 517 | F |
| 518 | A |
| 519 | A |
| 520 | A |
| 521 | A |
| 522 | A |
| 523 | A |
| 524 | A |
| 525 | A |
| 526 | A |
| 527 | A |
| 528 | A |
| 529 | F |
| 530 | F |
| 531 | C |
| 532 | F |
| 533 | D |
| 534 | A |
| 535 | C |
| 536 | F |
| 537 | F |
| 538 | F |
| 539 | A |
| 540 | C |
| 541 | A |
| 542 | A |
| 543 | A |
| 544 | A |
| 545 | A |
| 546 | A |
| 547 | A |
| 548 | A |
| 549 | A |
| 550_P1 | A |
| 550_P2 | A |
| 551 | C |
| 552 | C |
| 553 | A |
| 554 | A |
| 555 | F |
| 556 | A |
| 557 | F |
| 558 | F |
| 559 | F |
| 560 | A |
| 561 | A |
| 562 | A |
| 563 | A |
| 564 | A |
| 565_P1 | F |
| 565_P2 | A |
| 566 | F |
| 567 | A |
| 568 | A |
| 569 | A |
| 570 | A |
| 571 | A |
| 572 | A |
| 573_P1 | A |
| 573_P2 | A |
| 574 | F |
| 575 | F |
| 576 | F |
| 577 | F |
| 578 | D |
| 579 | C |
| 580 | E |
| 58 | E |
| 582 | F |
| 583 | E |
| 584 | F |
| 585 | F |
| 586 | A |
| 587 | A |
| 588 | A |
| 589 | A |
| 590 | A |
| 591 | A |
| 592 | A |
| 593 | A |
| 594 | A |
| 595 | A |
| 596 | A |
| 597_P1 | E |
| 597_P2 | A |
| 598 | A |
| 599 | A |
| 600 | A |
| 601 | A |
| 602 | C |
| 603 | A |
| 604 | A |
| 605 | A |
| 606 | B |
| 607 | A |
| 608 | A |
| 609 | A |
| 610_P1 | A |
| 610_P2 | A |
| 611 | B |
| 612_P1 | F |
| 612_P2 | A |
| 613 | A |
| 614 | A |
| 615 | C |
| 616 | A |
| 617 | B |
| 618 | B |
| 619 | F |
| 620 | B |
| 621 | A |
| 622 | A |
| 623 | A |
| 624 | C |
| 625 | A |
| 626 | A |
| 627_P1 | A |
| 627_P2 | D |
| 628_P1 | A |
| 628_P2 | E |

### REFERENCES

1: Bachmaier, et. al. Nature (2000) 403:211-216
2. Chiang, et. al. Nature (2000) 403:216-220
3: Ronchi and Haas, Methods Mol Biol. (2012) 832: 197

1. A compound of formula (A): 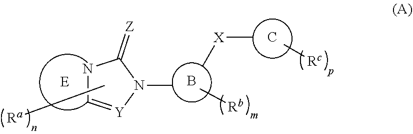
or pharmaceutically acceptable salts thereof,
wherein
Y is selected from the group ═C(H)—, ═C(R^{a})— or ═N—;
Z is ═O or ═S;
E is optionally substituted 5-6 membered heterocyclyl;
B is optionally substituted phenyl, optionally substituted 8-10 membered bicyclyl, or optionally substituted 5-6 membered heteroaryl;
C is optionally substituted 5-6 membered heterocyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from the group consisting of halogen, C₁-C₃ aliphatic, phenyl, 3-6-membered heteroaryl, 3-6-membered heterocylyl, and —(CH₂)(3-6-membered carbocyclyl);
each R^{a} is independently selected from the group consisting of L-Y, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR1R², —SO₂NH₂, —SO₂NR1R², —SO₂OH, —SO₂OR¹, —S(O)R¹, —S(O)₂R¹, —S(O)(NH)R¹, —S(O)(NR1)R¹, optionally substituted C1-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR1R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C1-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5;
m is 0, 1, 2, 3, or 4; and
p is 0, 1, 2, 3, or 4.

2. The compound of claim 1, wherein C is selected from the group consisting of optionally substituted triazolyl, optionally substituted pyrazolyl, optionally substituted isoxazolyl, optionally substituted thiazolyl, optionally substituted thiadizolyl, optionally substituted pyridinyl, optionally substituted pyrazinyl, optionally substituted pyrimidinyl, and optionally substituted pyridazinyl.

3. The compound of any of claims 1-2, wherein the compound is of Formula (B): 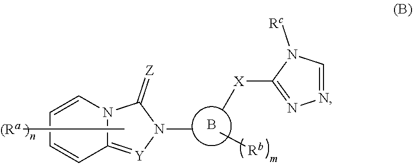
or pharmaceutically acceptable salts thereof.

4. The compound of any of claims 1-2, wherein the compound is of Formula (I): 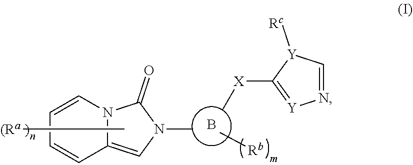
or pharmaceutically acceptable salts thereof.

5. The compound of any of claims 1-4, wherein the compound is of Formula (Ia) or (IIa): 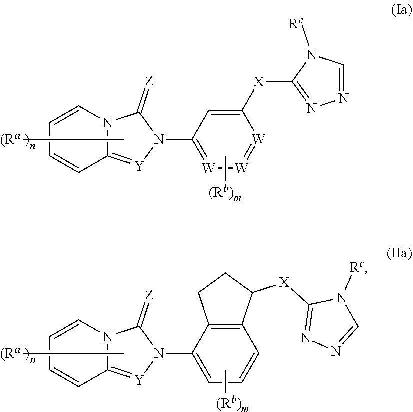
or a pharmaceutically acceptable salt thereof,
wherein each W is independently selected from N or C.

6. The compound of any of claims 1-5, wherein the compound is of formula (Ia1) or (IIa1): 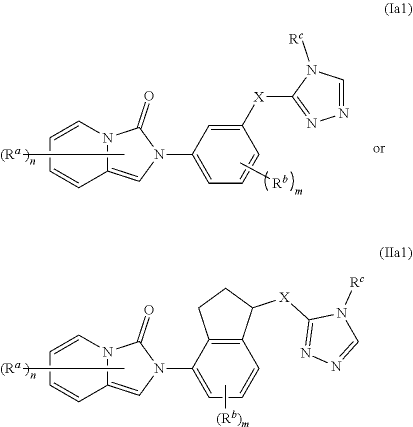
or a pharmaceutically acceptable salt thereof.

7. The compound of any of claims 1-5, wherein the compound is of Formula (Ia2), (Ia3), or (Ia4): 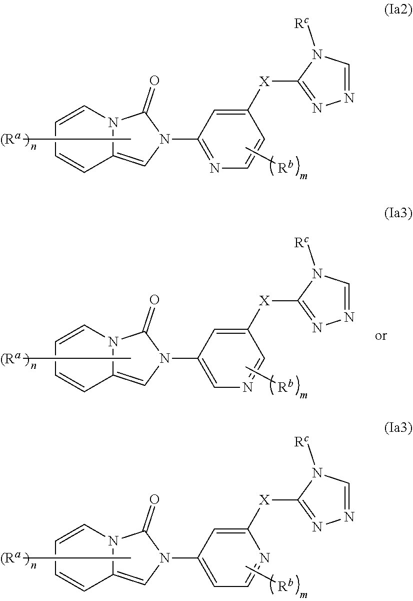
or a pharmaceutically acceptable salt thereof.

8. The compound of any of claims 1-5, wherein the compound is of formula (Ib) or (IIb): 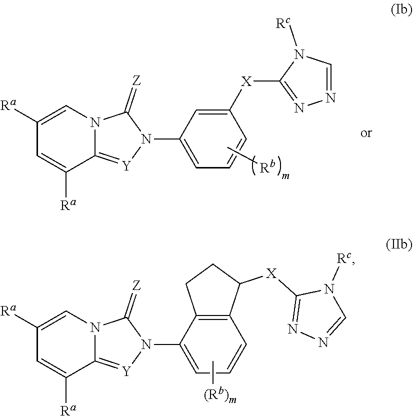
or pharmaceutically acceptable salts thereof,
wherein each W is independently selected from N or C.

9. The compound of any of claims 1-5, wherein the compound is of formula (Ic) or (IIc): 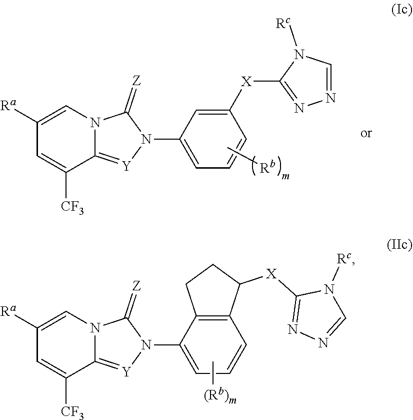
or pharmaceutically acceptable salts thereof.

10. The compound of any of claims 1-9, wherein R^{c} is optionally substituted C₁-C₃ aliphatic.

11. The compound of claim 10, wherein each R^{c} is independently selected from the group consisting of methyl, —CD₃, —CHF₂

12. The compound of claim 11, wherein R^{c} is methyl.

13. The compound of any of claims 1-12, wherein X is optionally substituted C₁-C₂ alkylene.

14. The compound of any of claims 1-12, wherein X is 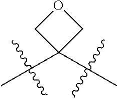 [SMILES: *C1(*)COC1]
or optionally substituted C₂ alkylene, wherein one methylene unit is replaced with

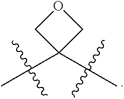 [SMILES: *C1(*)COC1]

15. The compound of any of claims 1-12, wherein X is selected from the group consisting of

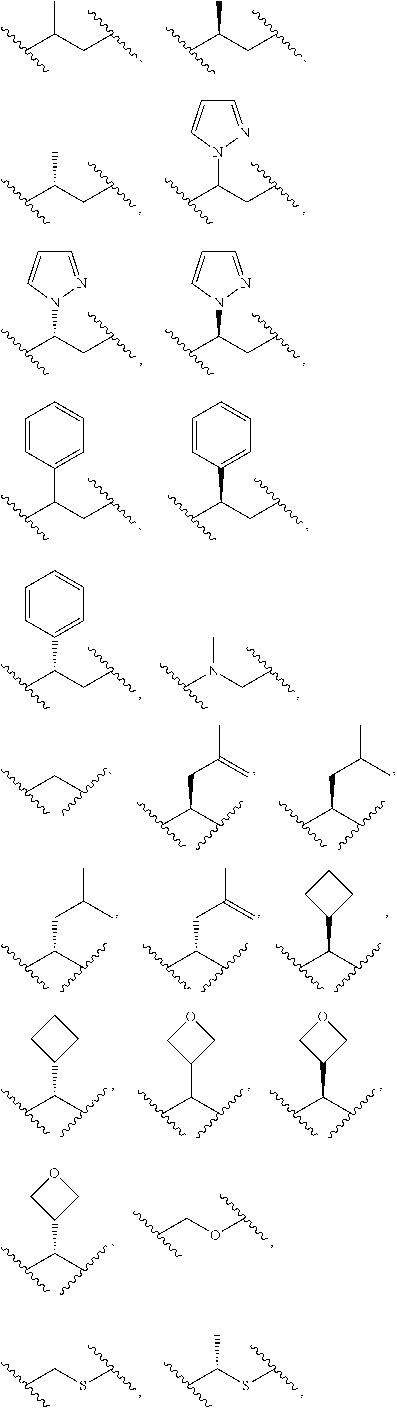

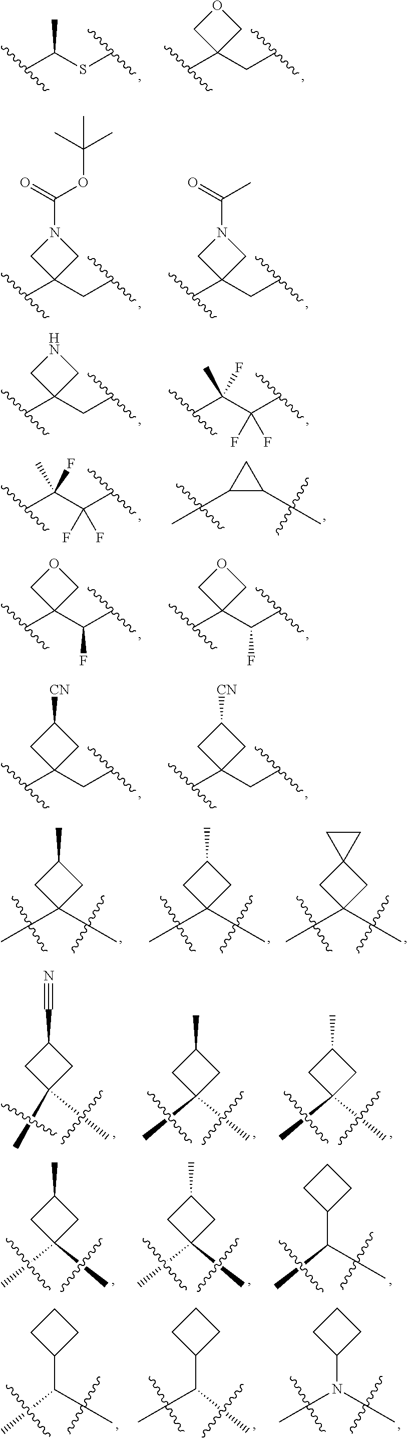

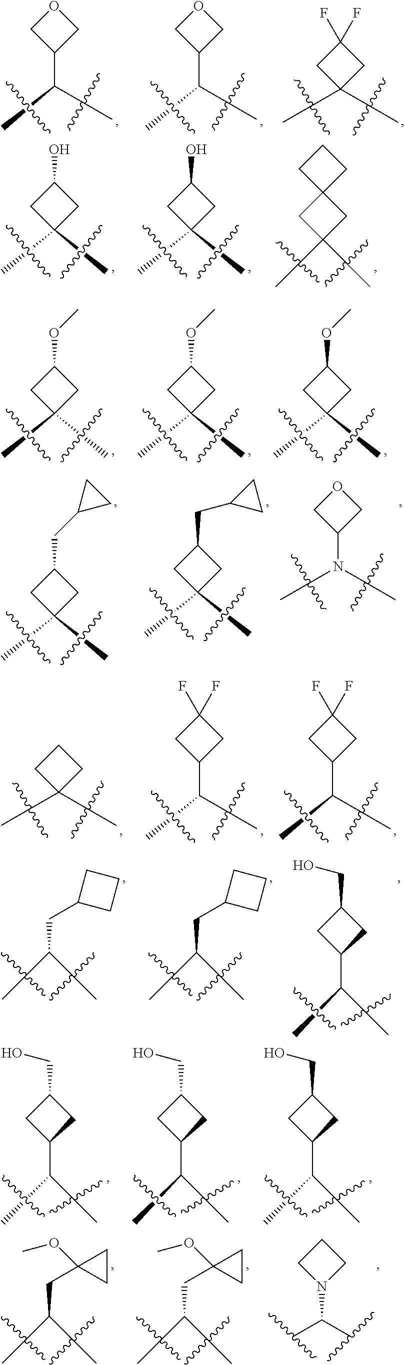

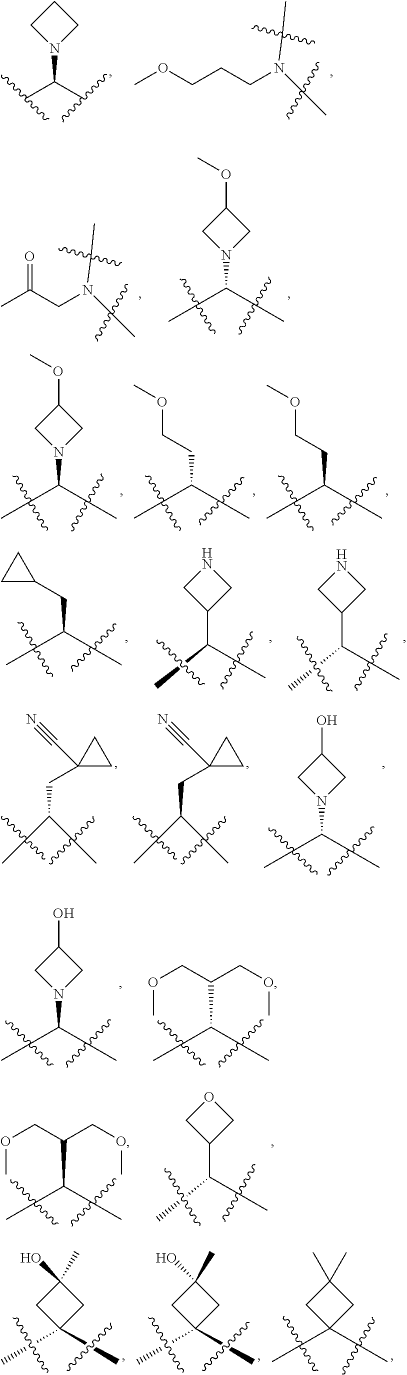

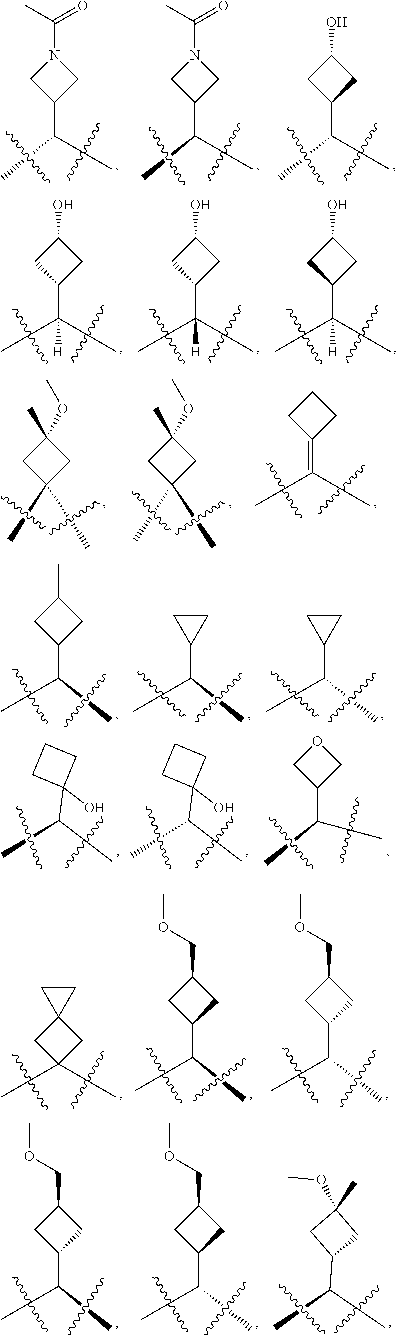

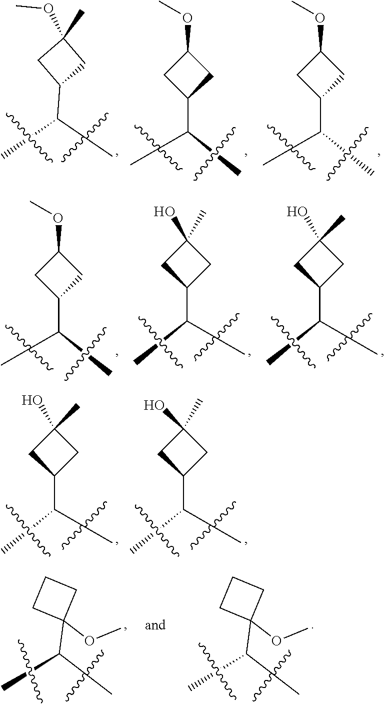

16. The compound of any of claims 1-15, wherein R^{a} is L-A.

17. The compound of claim 16, wherein L is —CH₂— or —CH(CH₃)—.

18. The compound of any of claims 16-17, wherein A is optionally substituted 3-6 membered heterocyclyl containing 1-4 heteroatoms each selected from the group consisting of N, O, and S.

19. The compound of any of claims 1-16, wherein R^{a} is selected from halogen, —CN, —C(O)R¹, —CO₂H, —CONR1R², optionally substituted C₁-C₆ aliphatic, and optionally substituted C₁-C₆ heteroalkyl.

20. The compound of any of claims 1-16, wherein each R^{a} is independently selected from the group consisting of halogen, —CN, —CO₂H, —CHO, —CHF₂, —CF₃, —OMe, —S(O)₂NHMe,

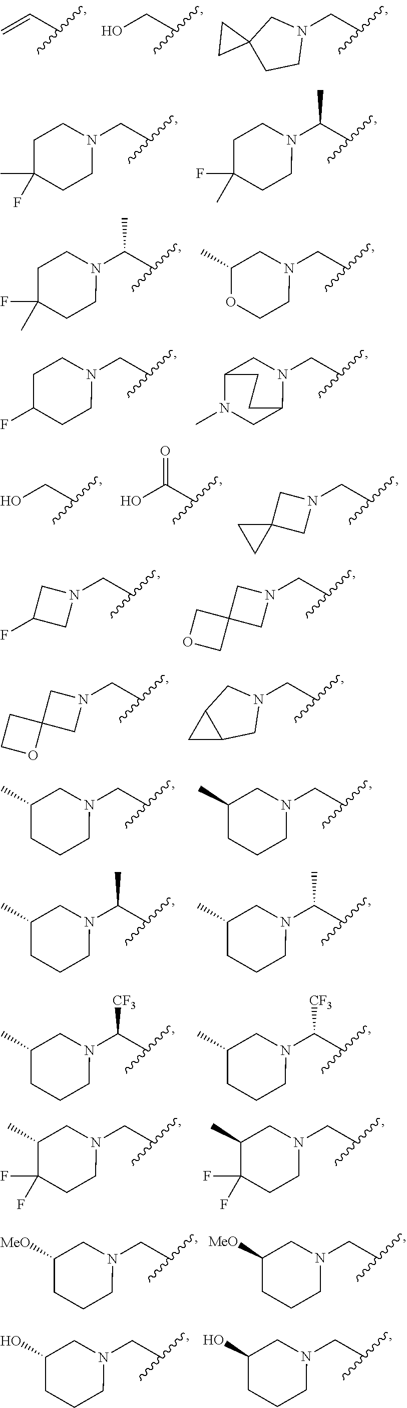

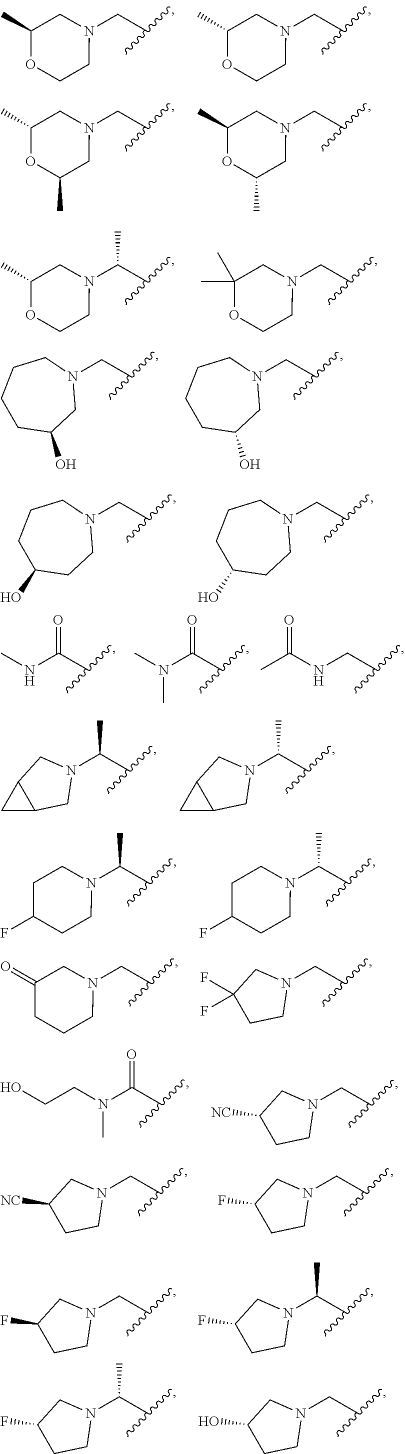

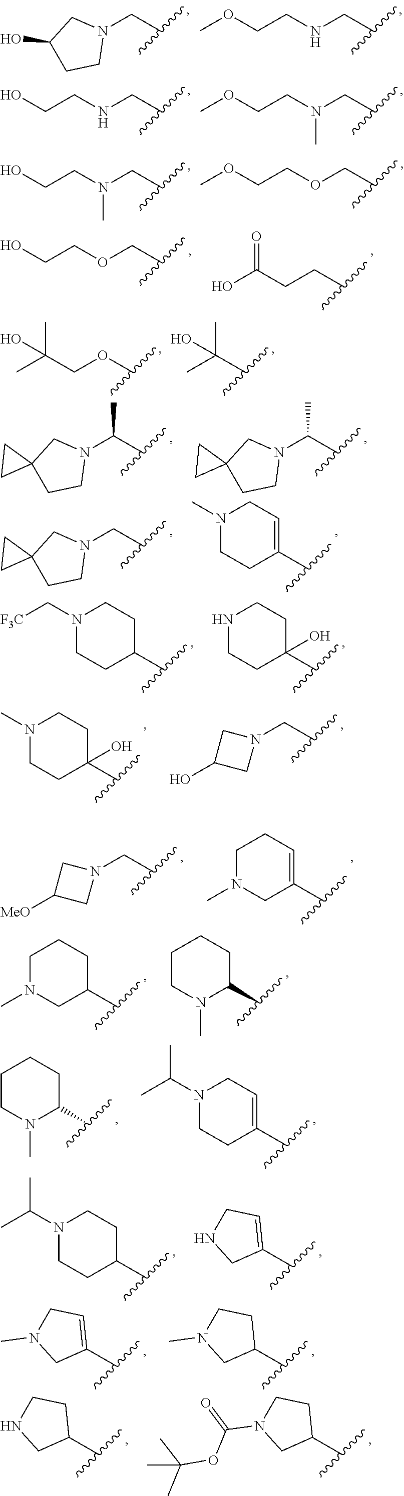

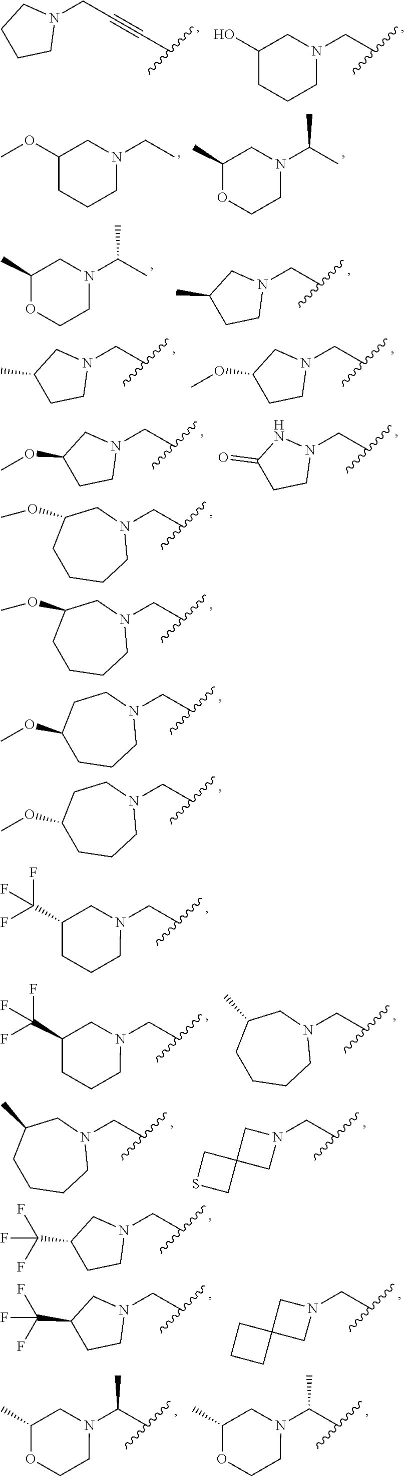

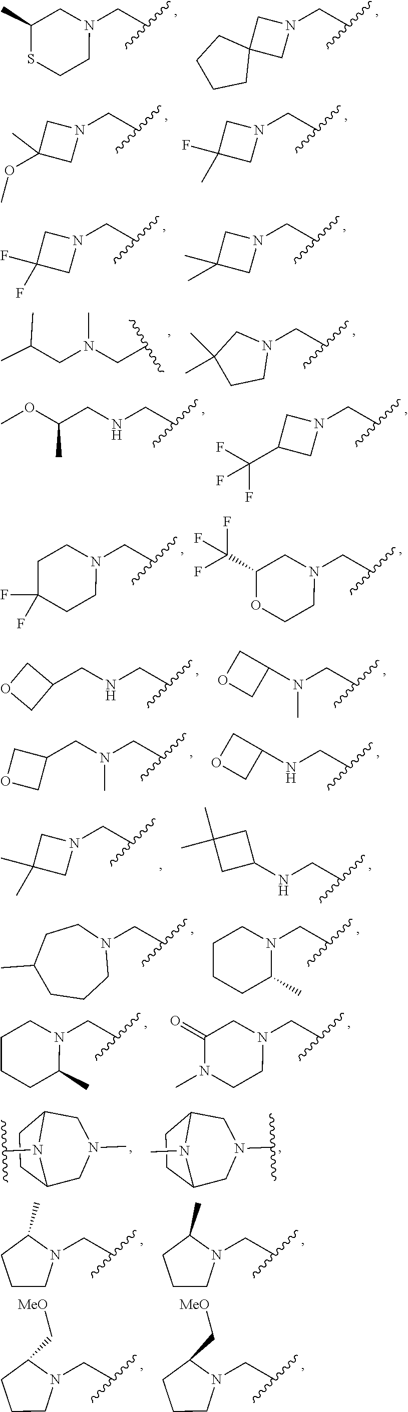

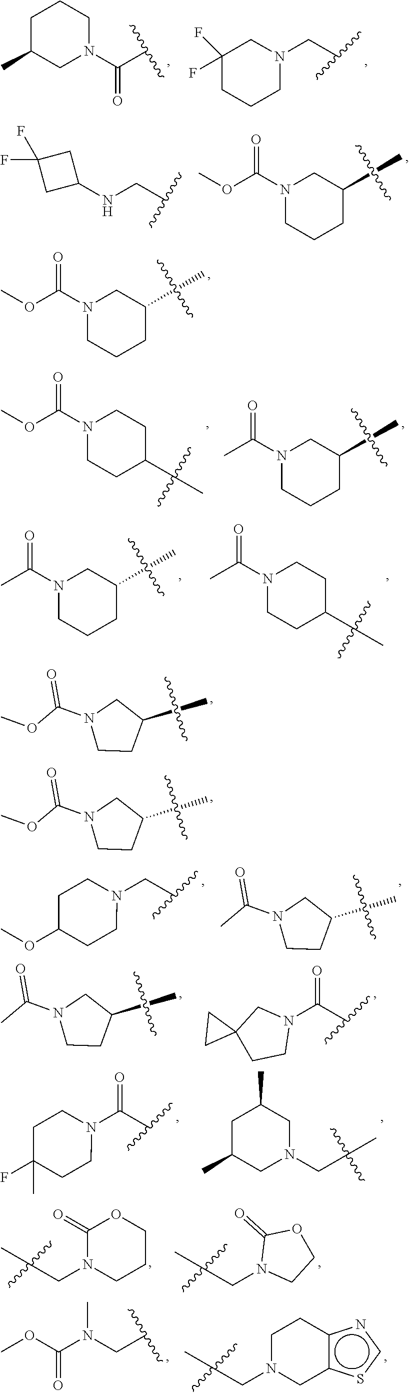

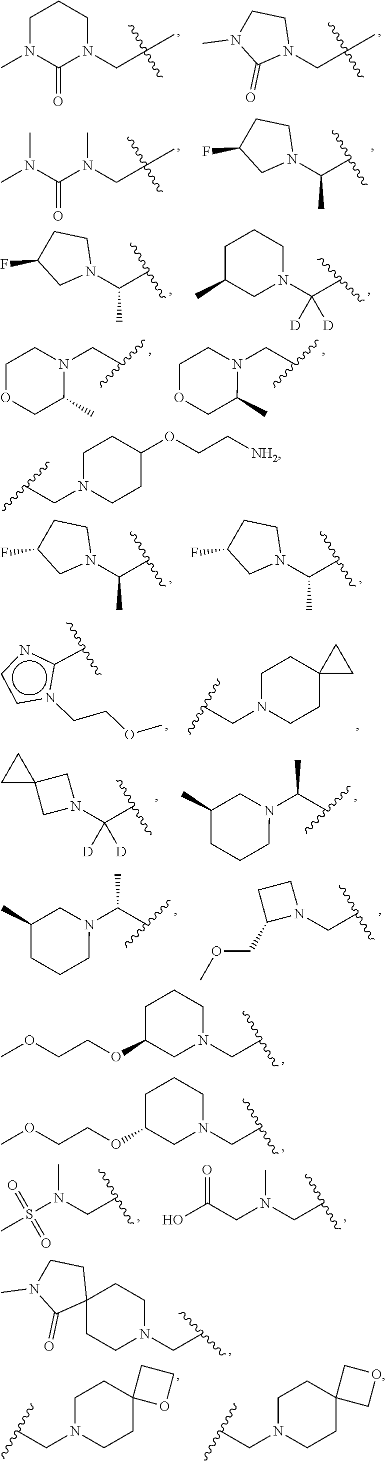

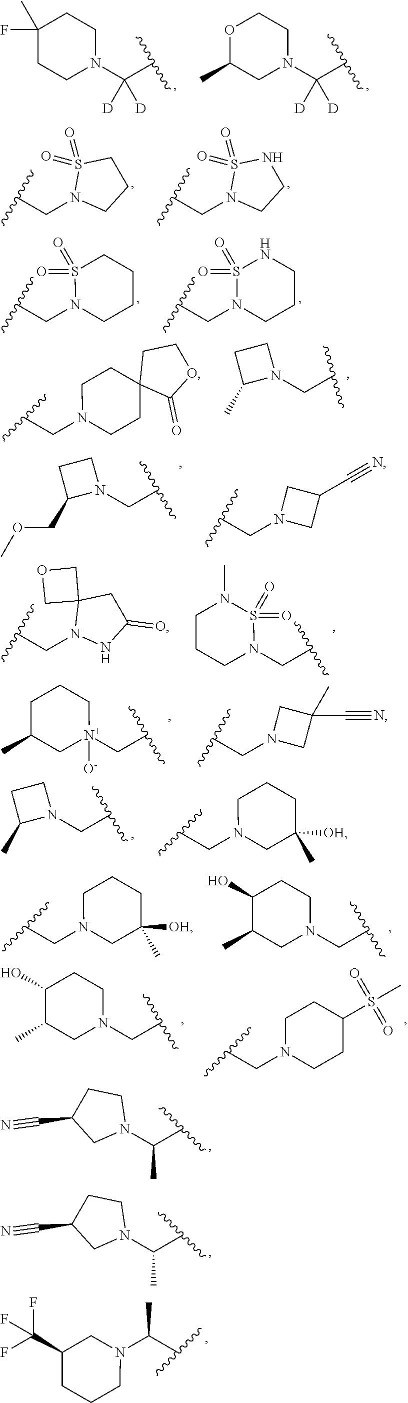

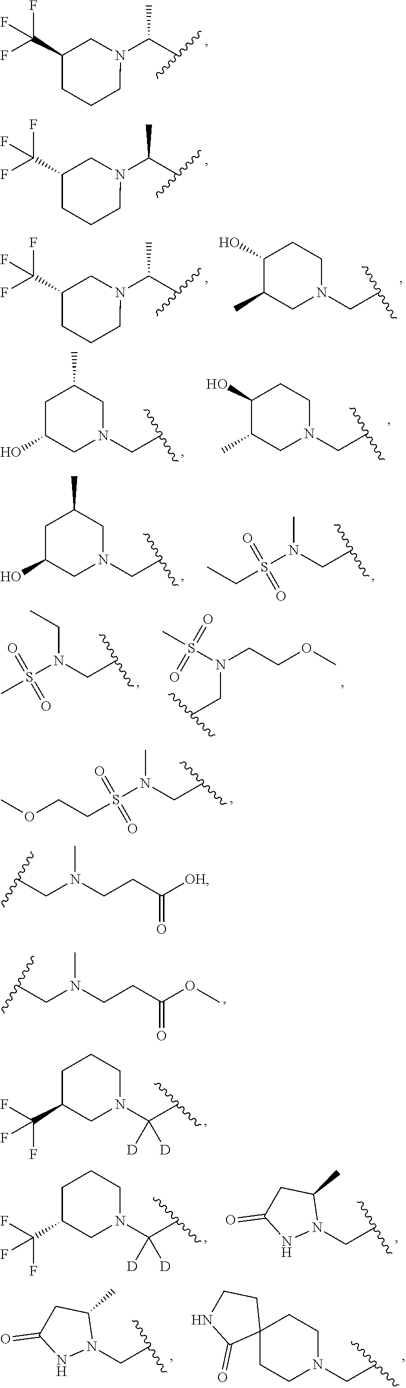

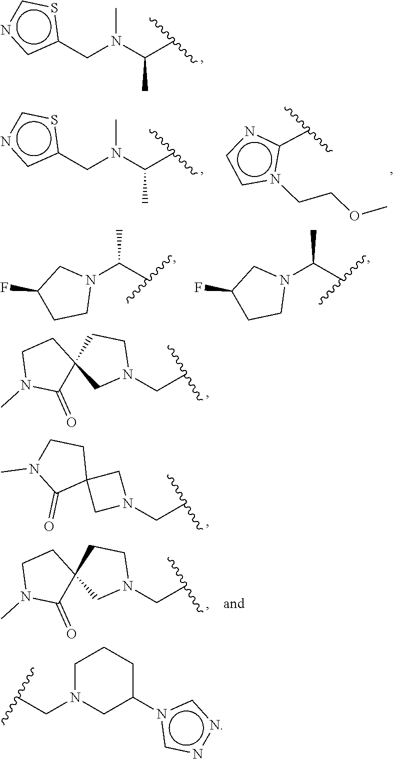

21. A compound selected from the group consisting of

| Cmpd | |
|---|---|
| No. | Structure |
| 1 | 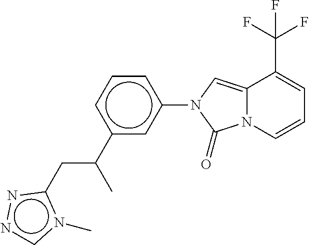 |
| 2 | 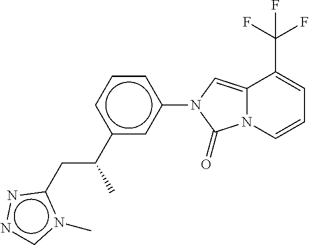 |
| 3 | 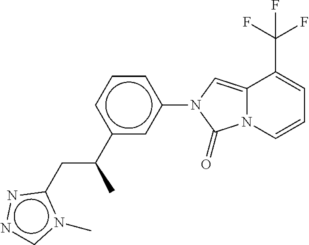 |
| 4 | 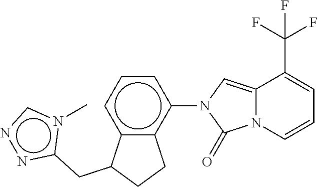 |
| 5 | 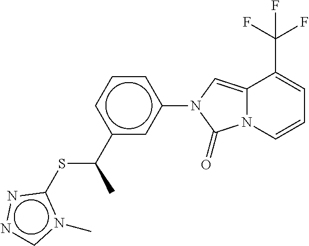 |
| 6 | 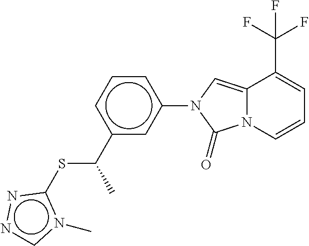 |
| 7 | 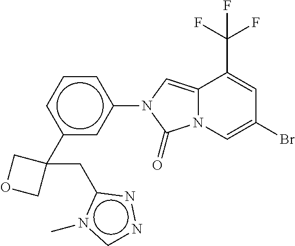 |
| 8 | 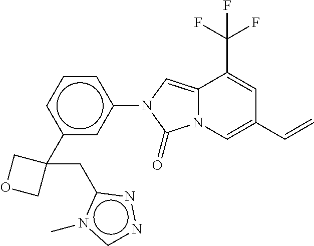 |
| 9 | 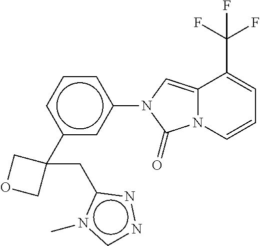 |
| 10 | 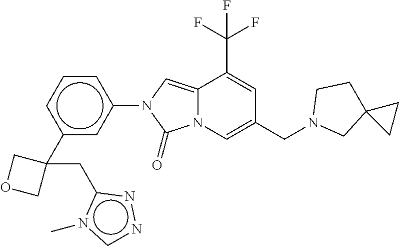 |
| 11 | 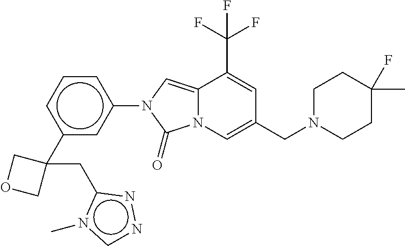 |
| 12 | 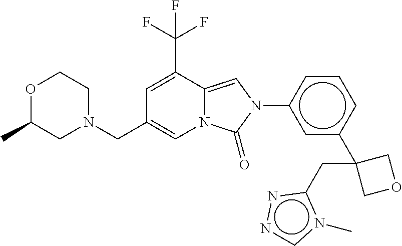 |
| 13 | 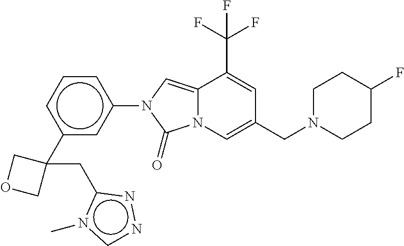 |
| 14 | 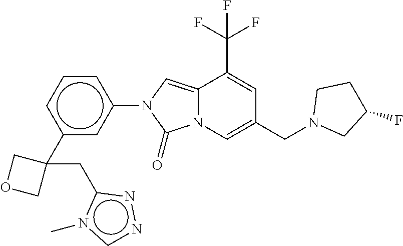 |
| 15 | 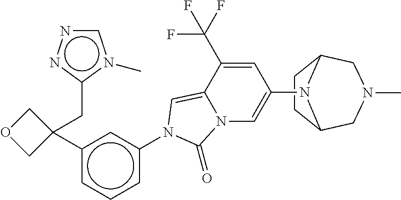 |
| 16 | 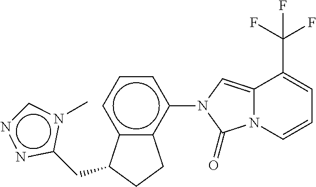 |
| 17 | 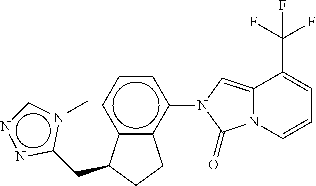 |
| 18 | 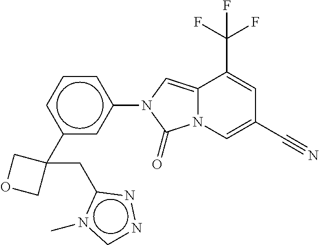 |
| 19 | 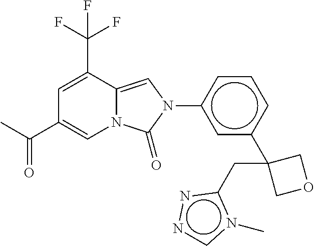 |
| 20 | 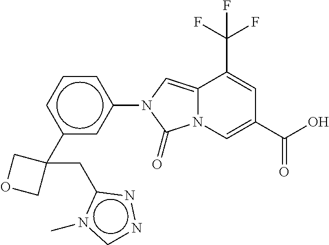 |
| 21 | 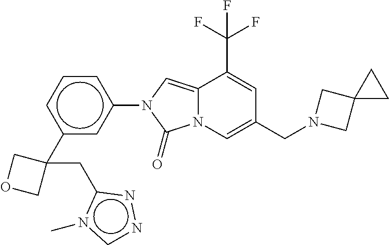 |
| 22 | 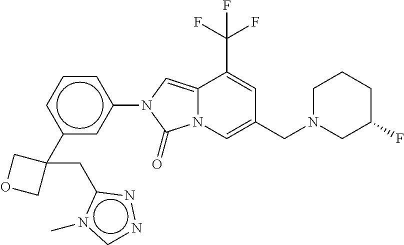 |
| 23 | 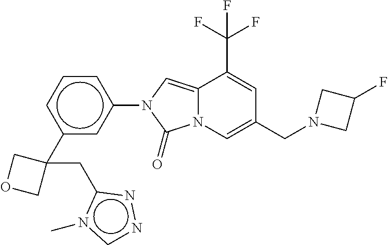 |
| 24 | 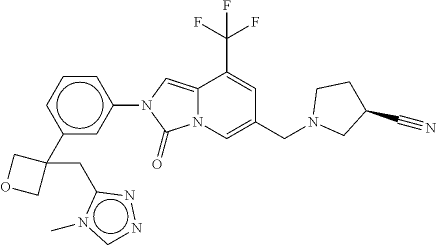 |
| 25 | 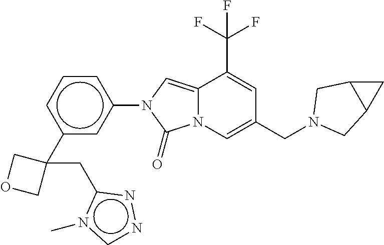 |
| 26 | 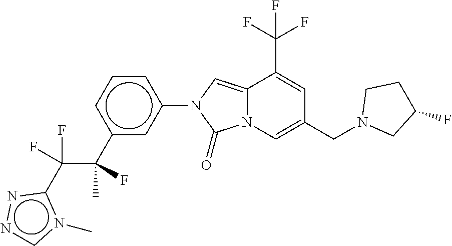 |
| 27 | 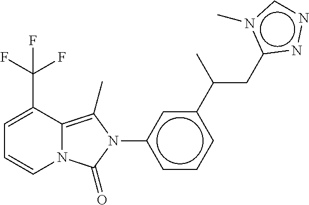 |
| 28 | 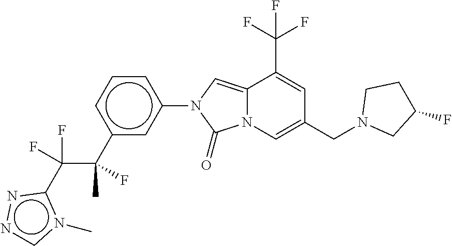 |
| 29 | 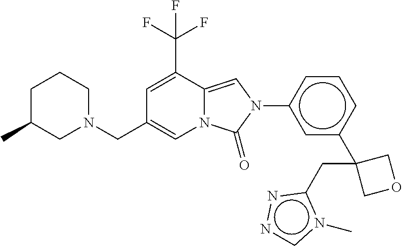 |
| 30 | 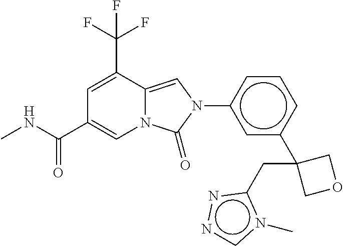 |
| 31 | 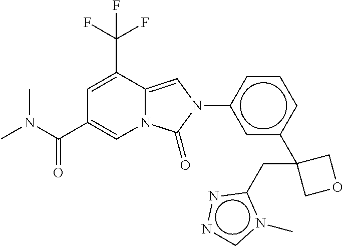 |
| 32 | 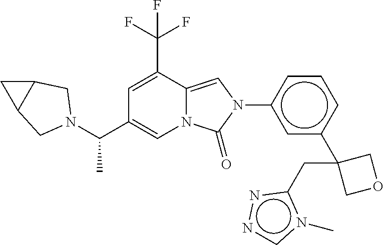 |
| 33 | 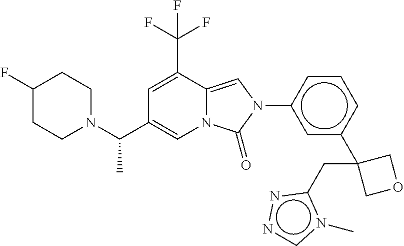 |
| 34 | 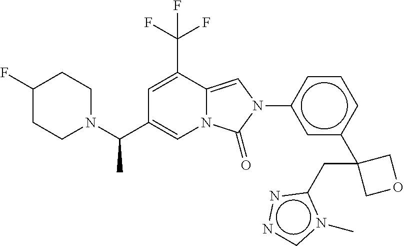 |
| 35 | 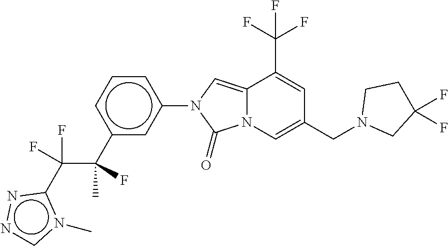 |
| 36 | 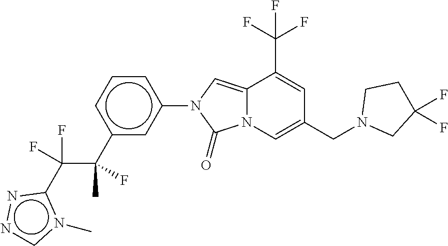 |
| 37 | 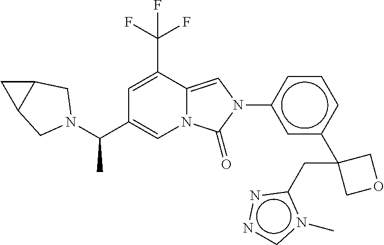 |
| 38 | 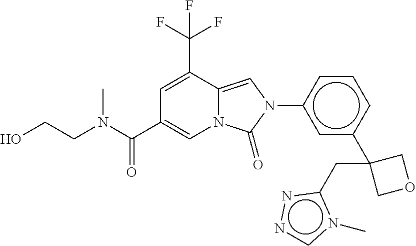 |
| 39 | 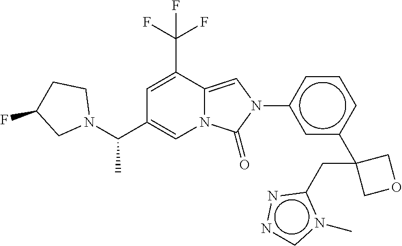 |
| 40 | 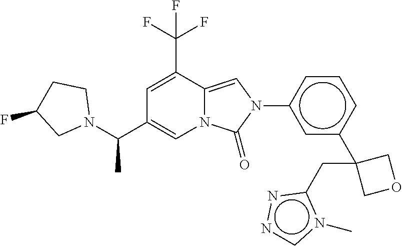 |
| 41 | 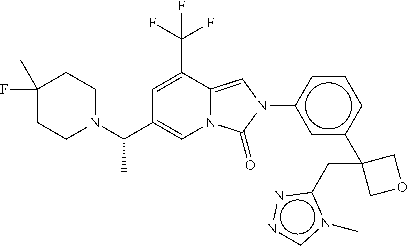 |
| 42 | 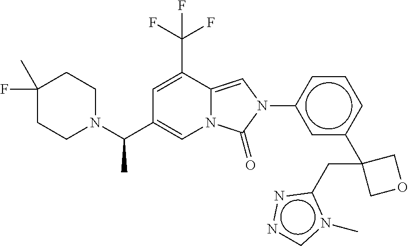 |
| 43 | 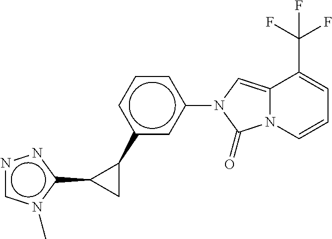 |
| 44 | 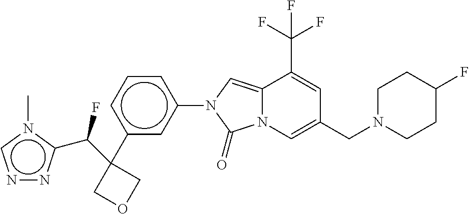 |
| 45 | 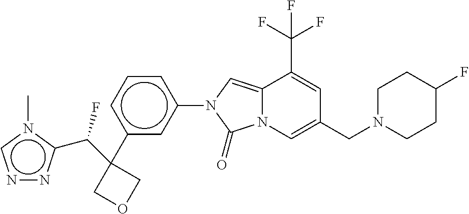 |
| 46 | 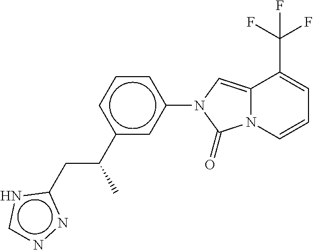 |
| 47 | 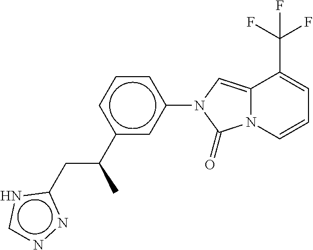 |
| 48 | 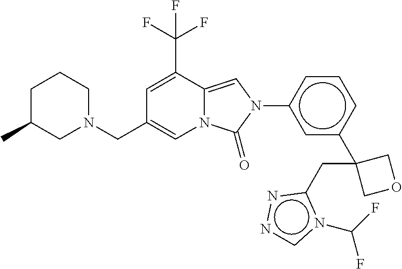 |
| 49 | 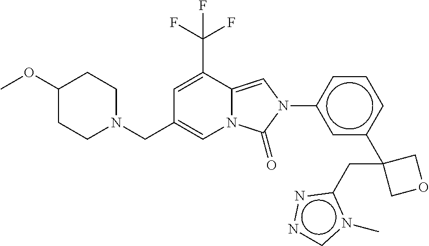 |
| 50 | 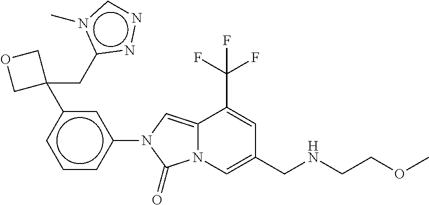 |
| 51 | 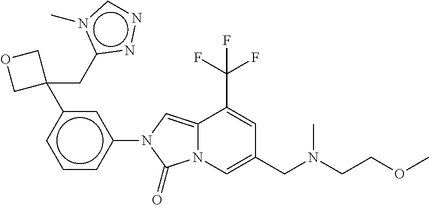 |
| 52 | 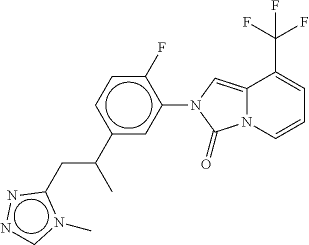 |
| 53 | 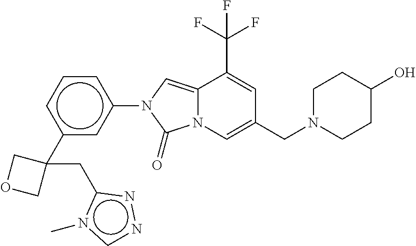 |
| 54 | 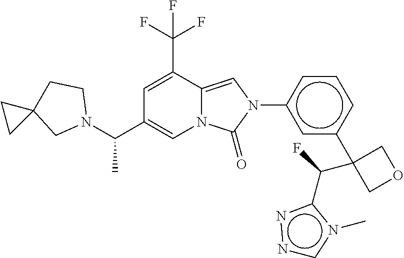 |
| 55 | 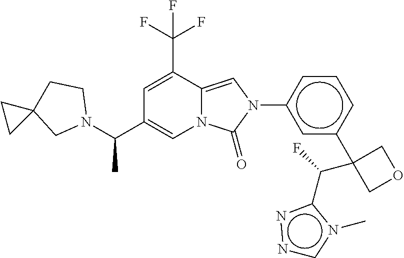 |
| 56 | 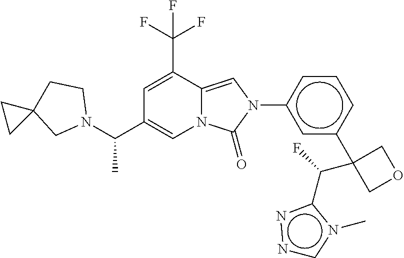 |
| 57 | 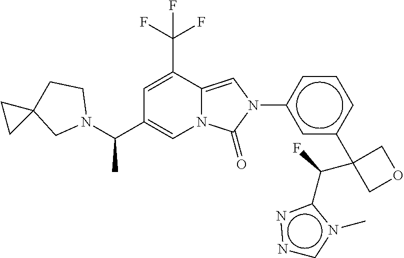 |
| 58 | 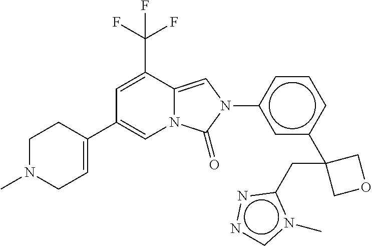 |
| 59 | 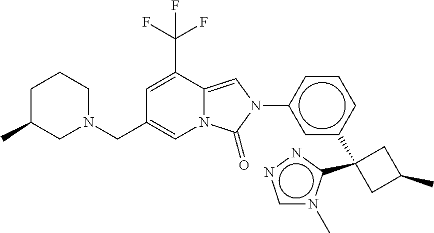 |
| 60 | 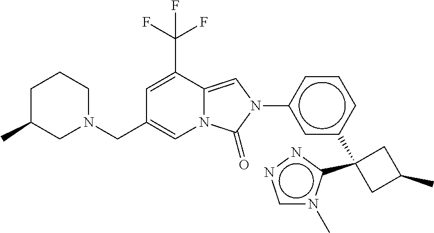 |
| 61 | 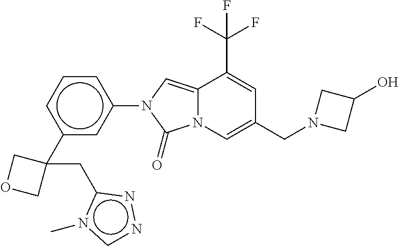 |
| 62 | 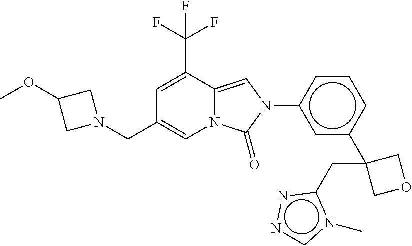 |
| 63 | 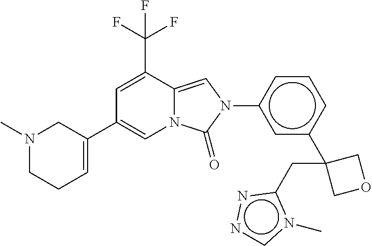 |
| 64 | 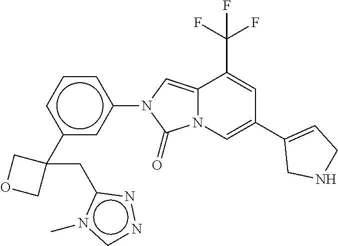 |
| 65 | 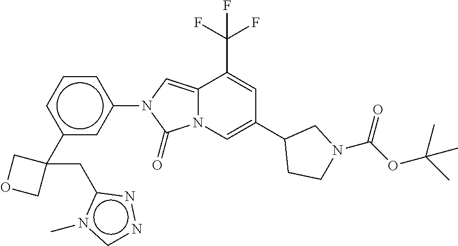 |
| 66 | 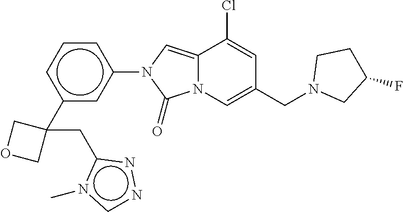 |
| 67 | 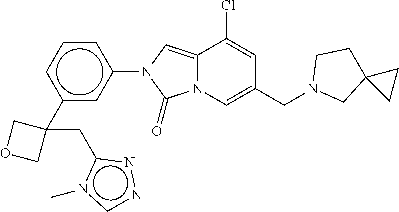 |
| 68 | 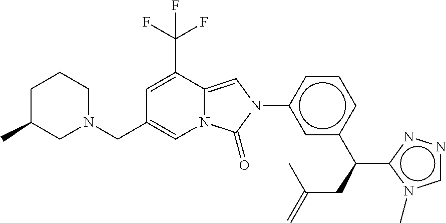 |
| 69 | 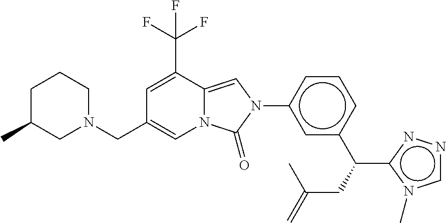 |
| 70 | 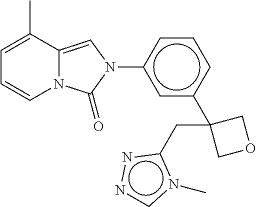 |
| 71 | 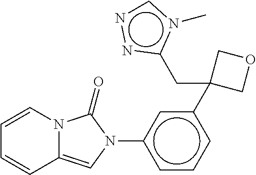 |
| 72 | 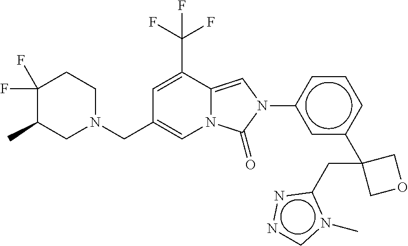 |
| 73 | 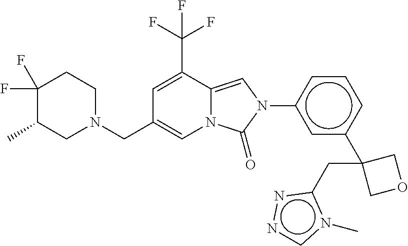 |
| 74 | 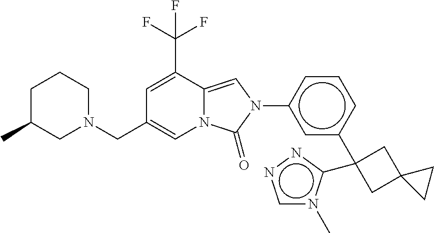 |
| 75 | 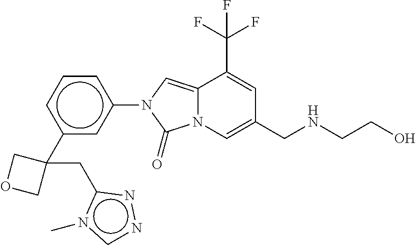 |
| 76 | 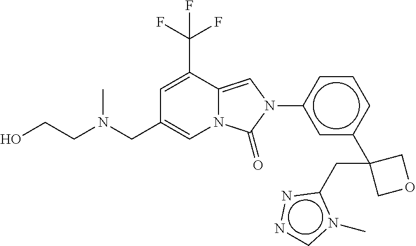 |
| 77 | 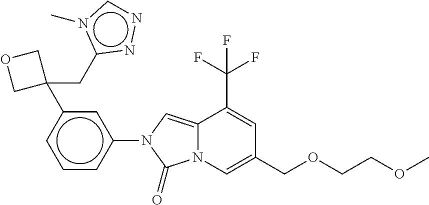 |
| 78 | 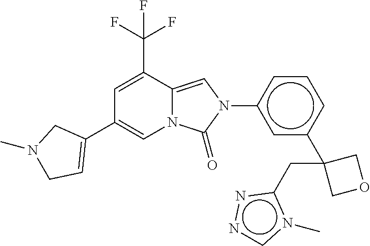 |
| 79 | 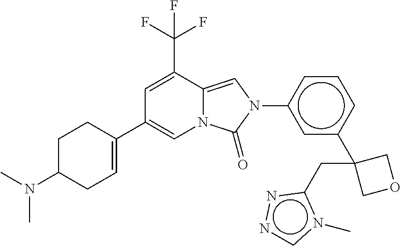 |
| 80 | 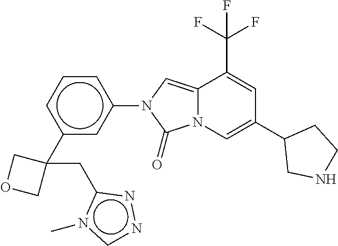 |
| 81 | 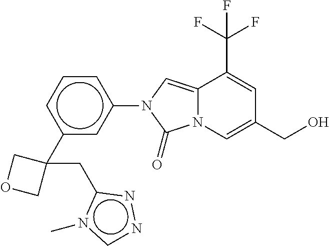 |
| 82 | 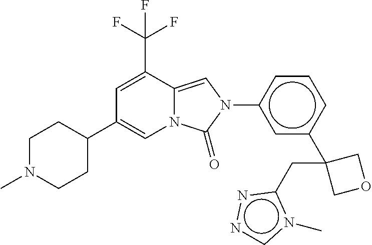 |
| 83 | 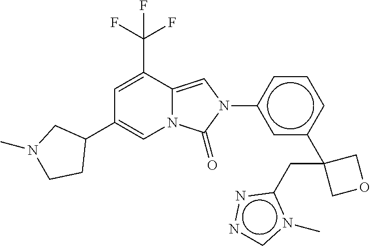 |
| 84 | 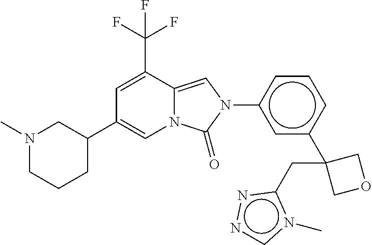 |
| 85 | 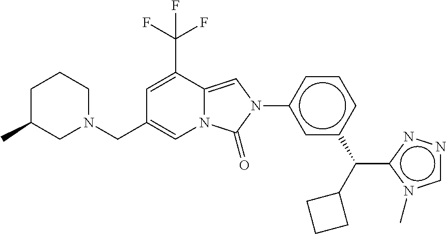 |
| 86 | 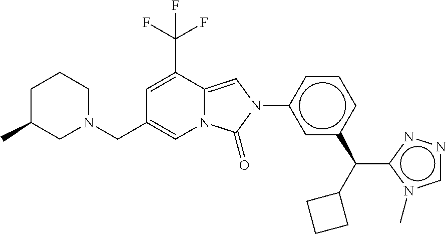 |
| 87 | 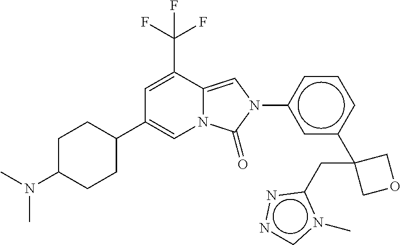 |
| 88 | 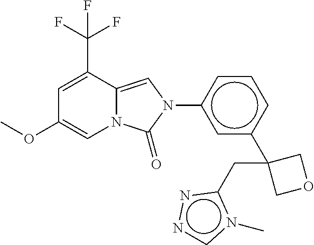 |
| 89 | 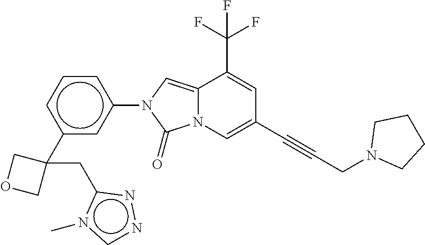 |
| 90 | 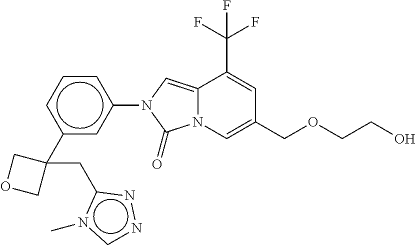 |
| 91 | 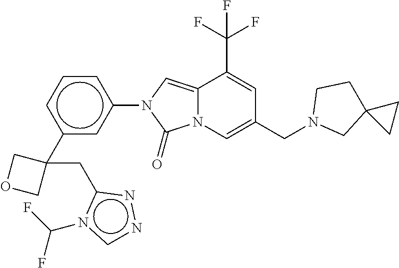 |
| 92 | 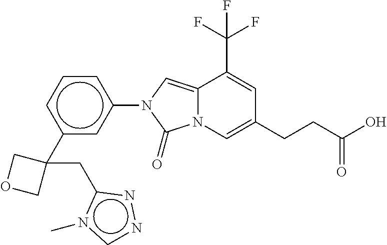 |
| 93 | 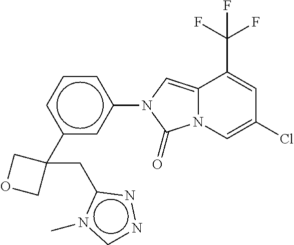 |
| 94 | 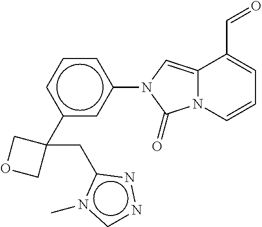 |
| 95 | 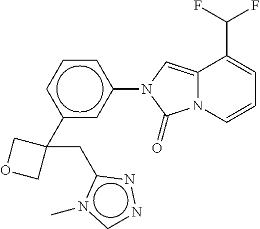 |
| 96 | 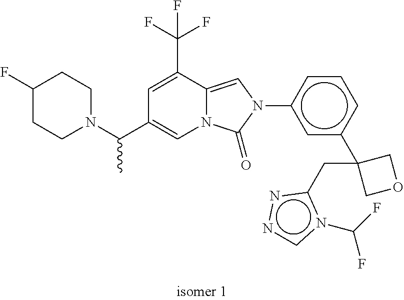 |
| 97 | 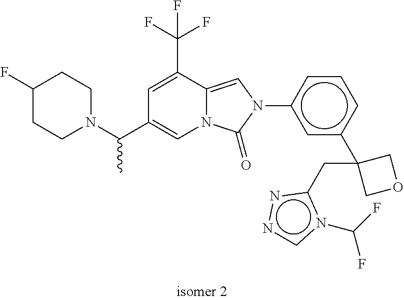 |
| 98 | 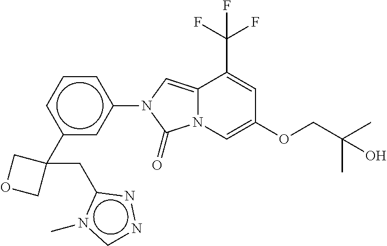 |
| 99 | 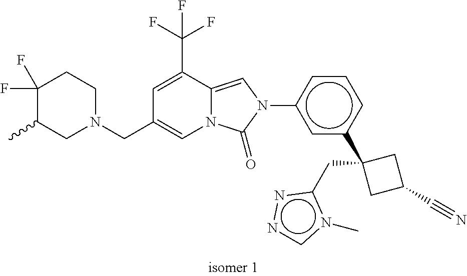 |
| 100 | 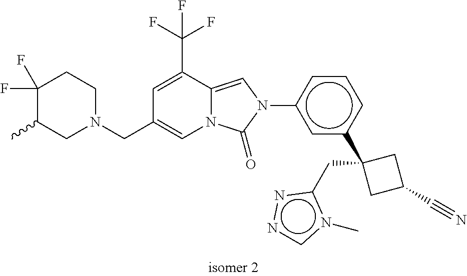 |
| 101 | 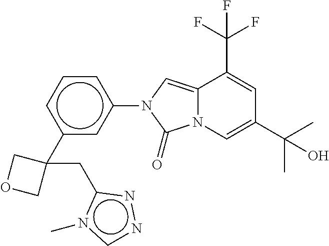 |
| 102 | 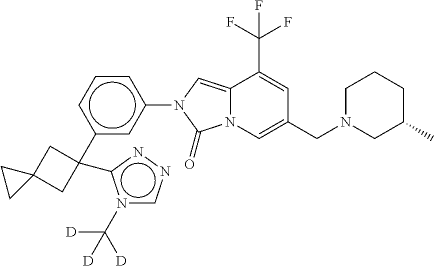 |
| 103 | 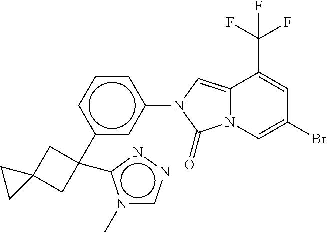 |
| 104 | 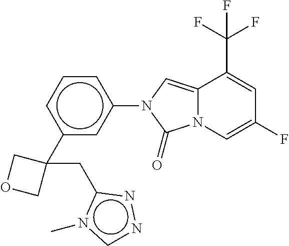 |
| 105 | 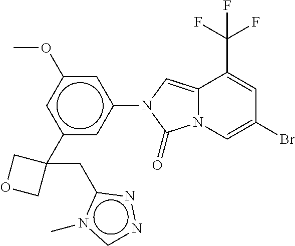 |
| 106 | 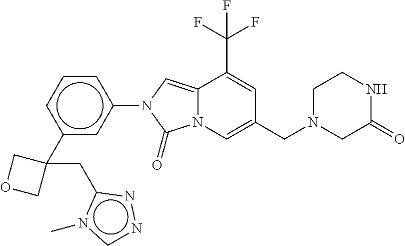 |
| 107 | 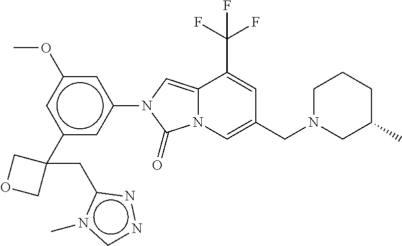 |
| 108 | 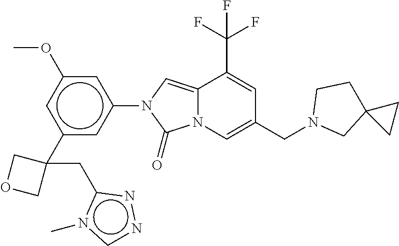 |
| 109 | 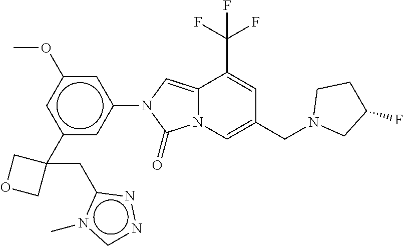 |
| 110 | 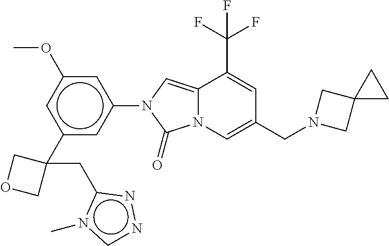 |
| 111 | 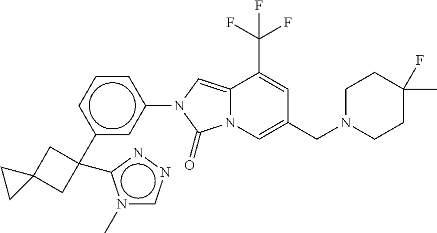 |
| 112 | 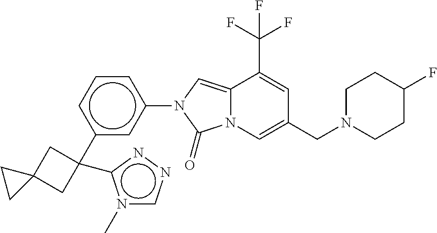 |
| 113 | 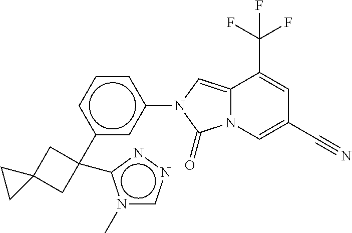 |
| 114 | 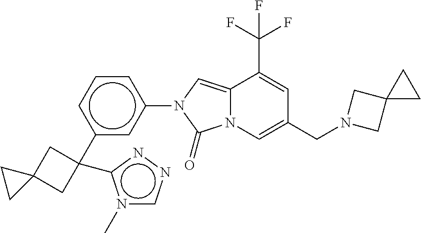 |
| 115 | 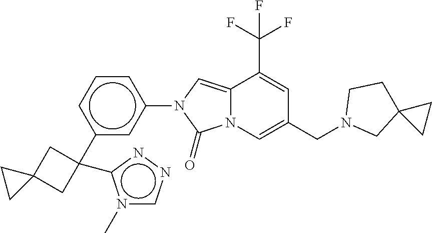 |
| 116 | 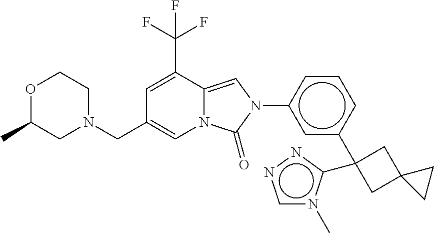 |
| 117 | 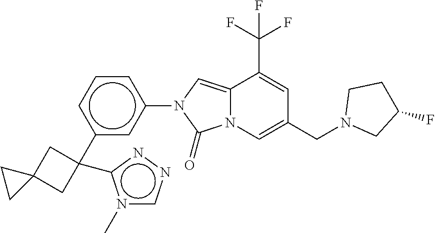 |
| 118 | 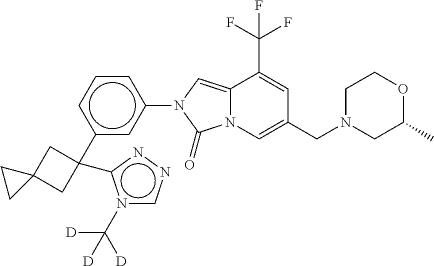 |
| 119 | 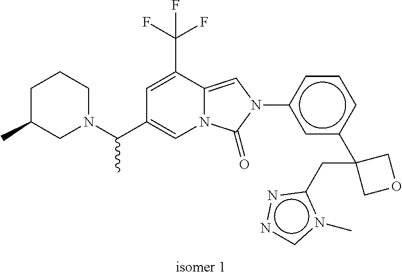 |
| 120 | 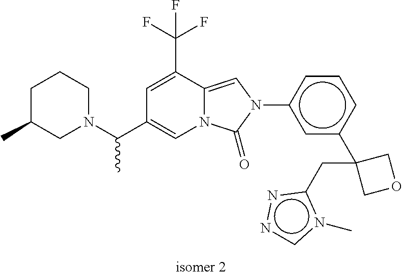 |
| 121 | 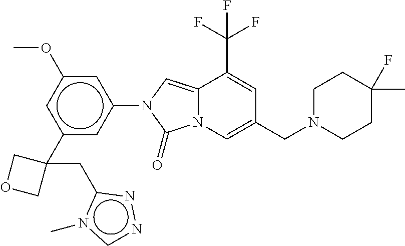 |
| 122 | 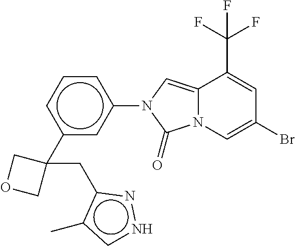 |
| 123 | 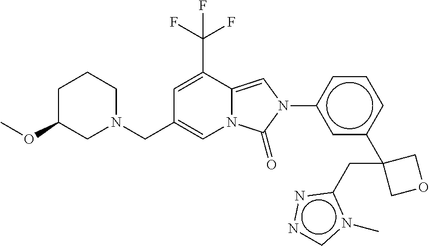 |
| 124 | 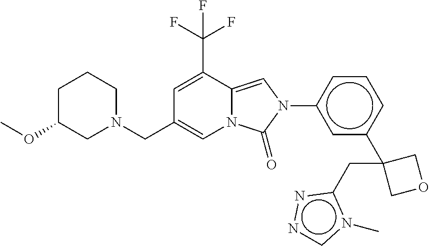 |
| 125 | 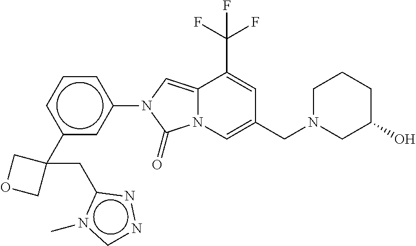 |
| 126 | 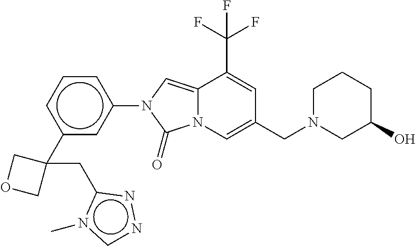 |
| 127 | 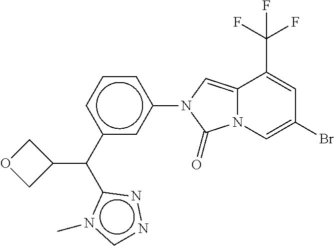 |
| 128 | 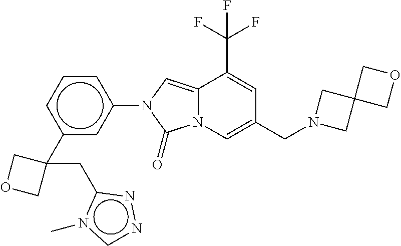 |
| 129 | 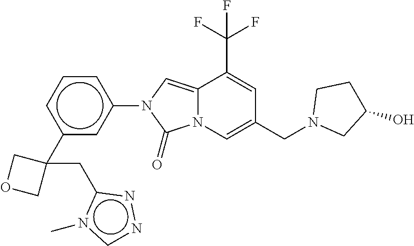 |
| 130 | 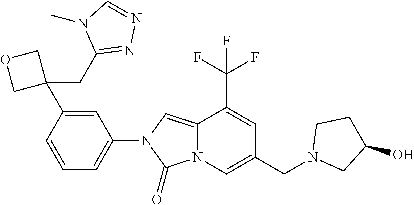 [SMILES: Cn1cnnc1CC1(c2cccc(-n3cc4c(C(F)(F)F)cc(CN5CC[C@@H](O)C5)cn4c3=O)c2)COC1] |
| 131 | 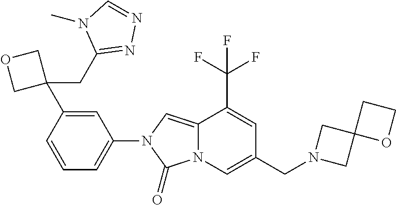 [SMILES: Cn1cnnc1CC1(c2cccc(-n3cc4c(C(F)(F)F)cc(CN5CC6(CCO6)C5)cn4c3=O)c2)COC1] |
| 132 | 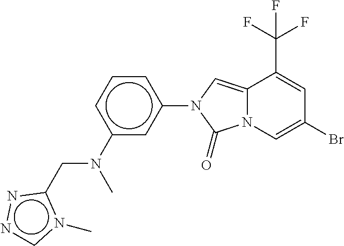 |
| 133 | 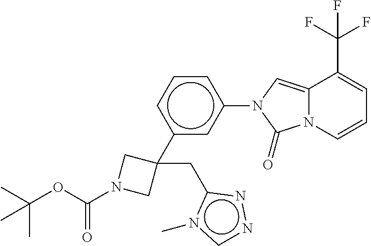 |
| 134 | 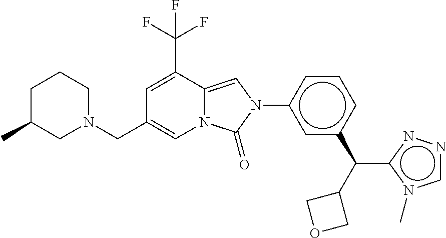 |
| 135 | 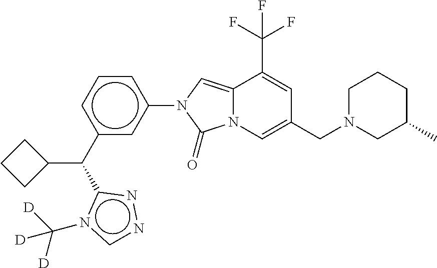 |
| 136 | 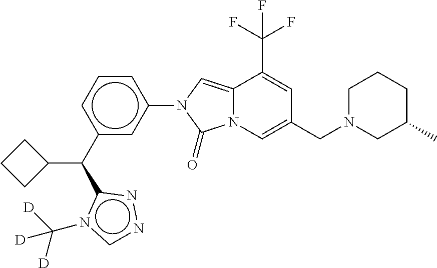 |
| 137 | 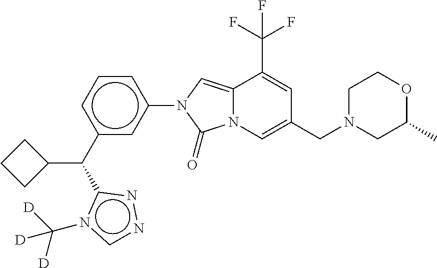 |
| 138 | 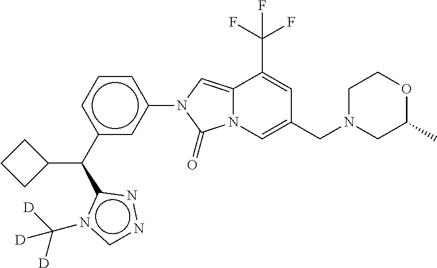 |
| 139 | 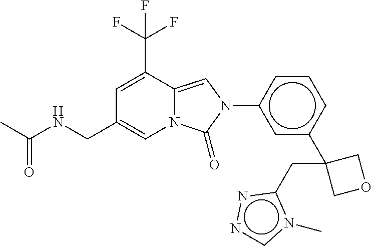 |
| 140 | 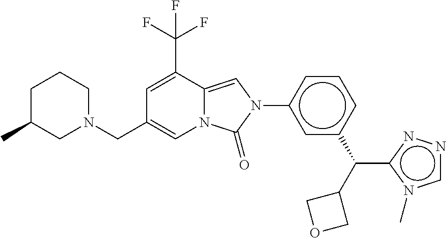 |
| 141 | 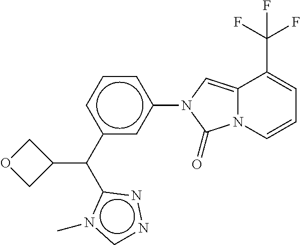 |
| 142 | 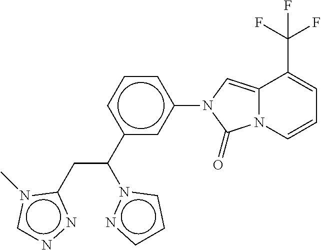 |
| 143 | 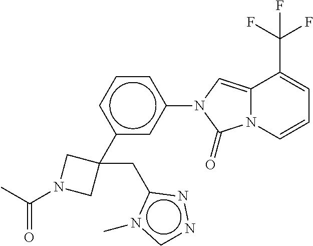 |
| 144 | 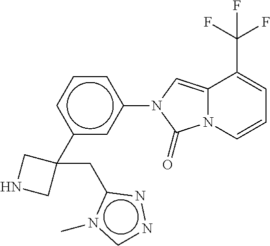 |
| 145 | 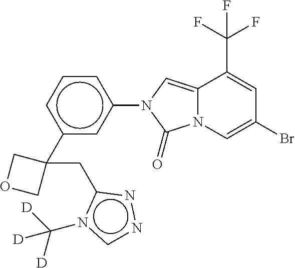 |
| 146 | 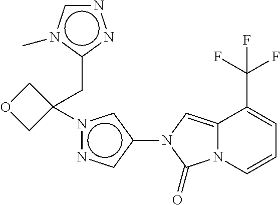 |
| 147 | 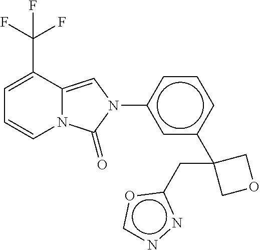 |
| 148 | 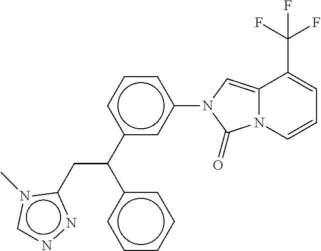 |
| 149 | 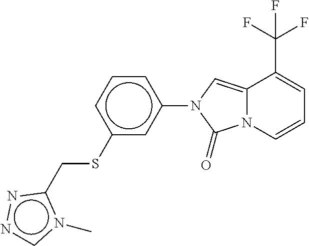 |
| 150 | 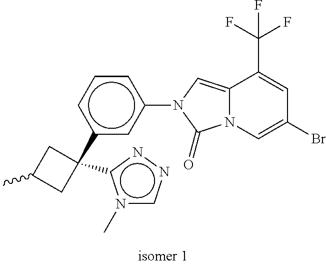 |
| 151 | 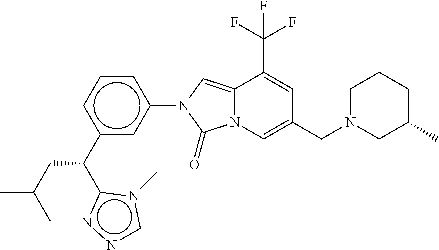 |
| 152 | 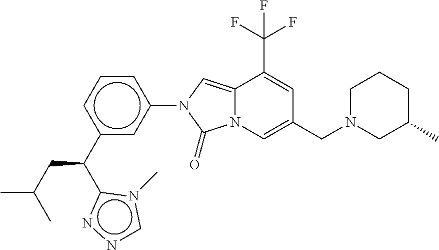 |
| 153 | 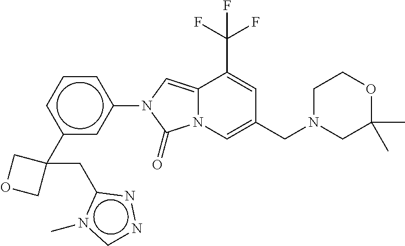 |
| 154 | 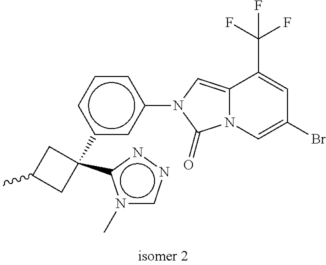 |
| 155 | 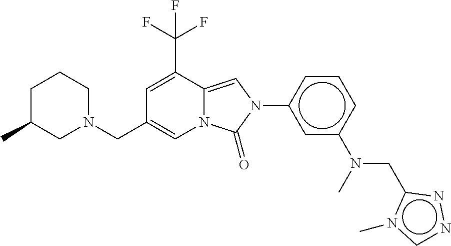 |
| 156 | 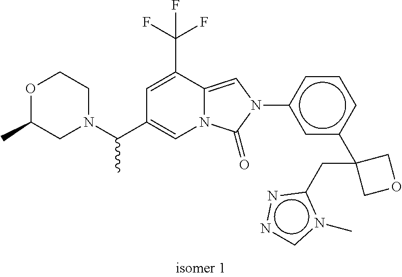 |
| 157 | 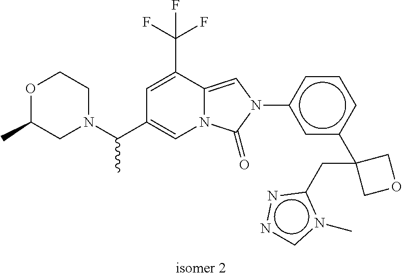 |
| 158 | 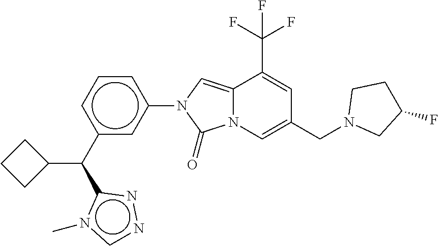 |
| 159 | 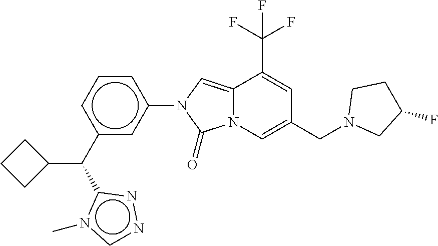 |
| 160 | 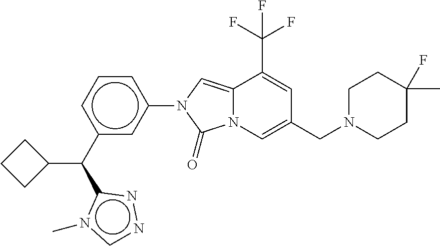 |
| 161 | 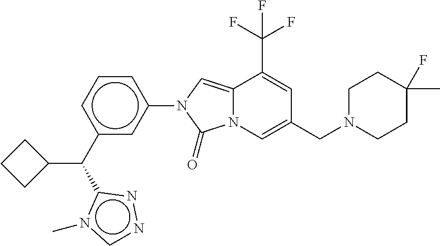 |
| 162 | 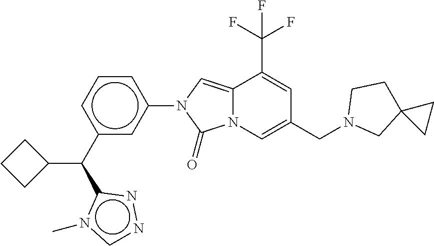 |
| 163 | 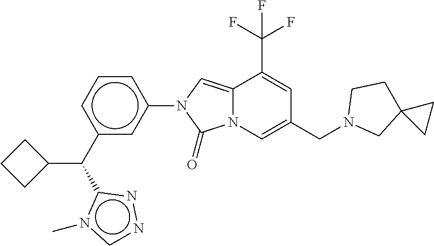 |
| 164 | 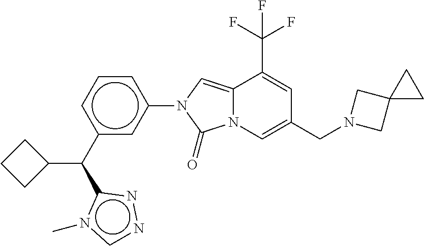 |
| 165 | 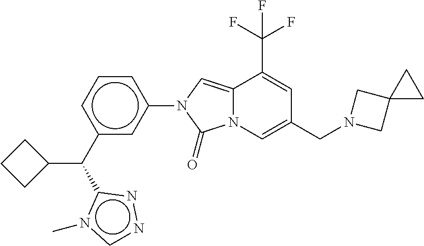 |
| 166 | 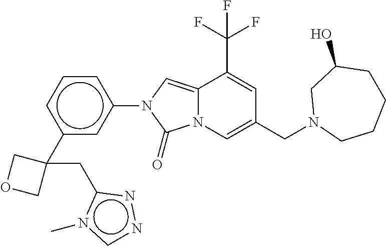 |
| 167 | 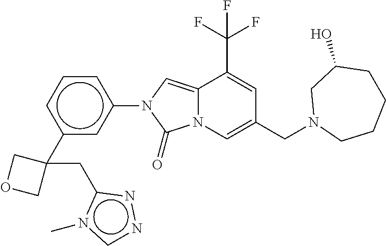 |
| 168 | 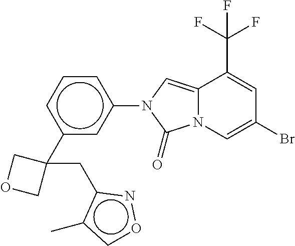 |
| 169 | 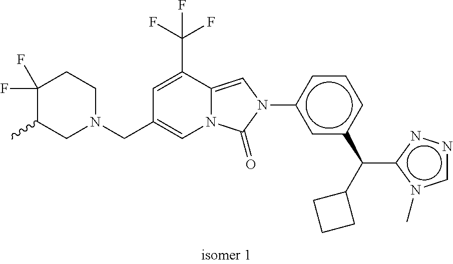 |
| 170 | 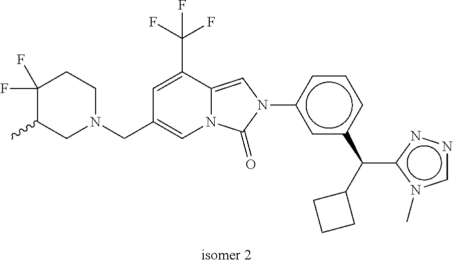 |
| 171 | 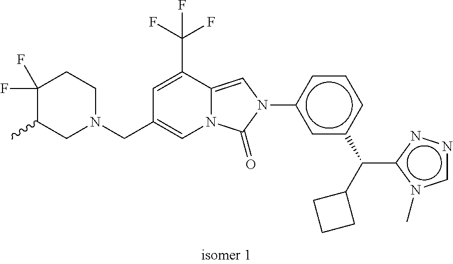 |
| 172 | 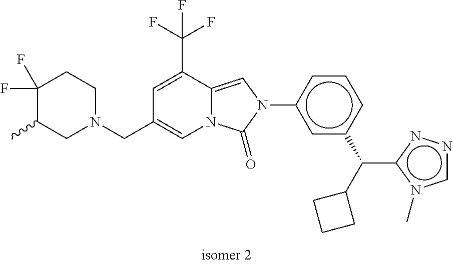 |
| 173 | 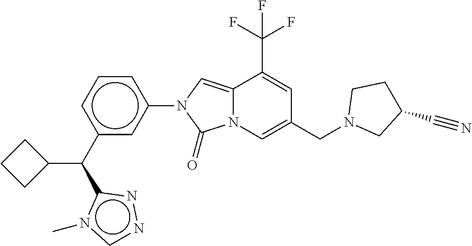 |
| 174 | 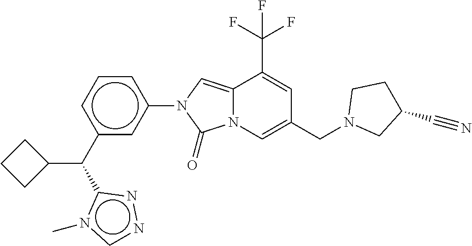 |
| 175 | 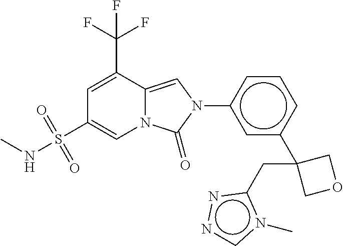 |
| 176 | 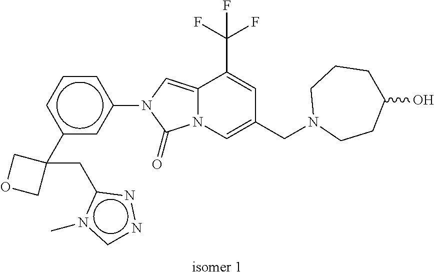 |
| 177 | 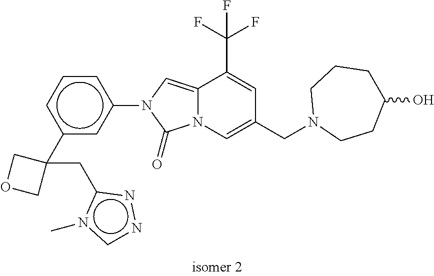 |
| 178 | 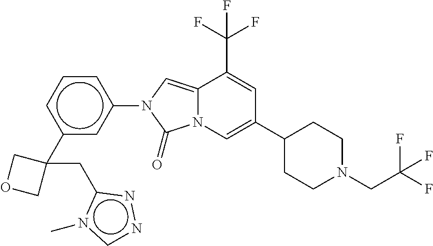 |
| 179 | 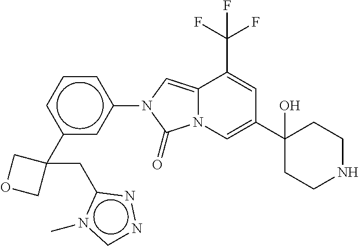 |
| 180 | 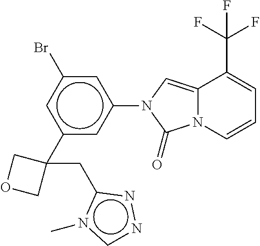 |
| 181 | 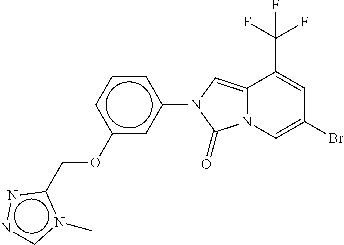 |
| 182 | 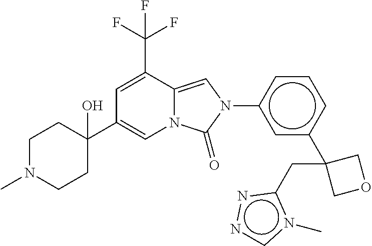 |
| 183 | 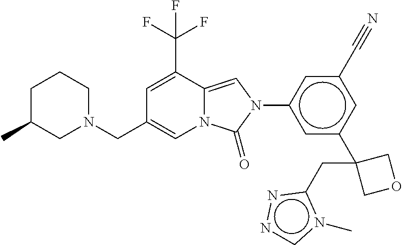 |
| 184 | 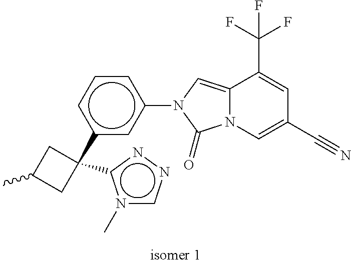 |
| 185 | 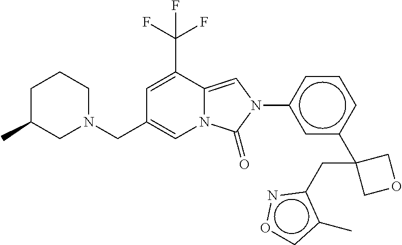 |
| 186 | 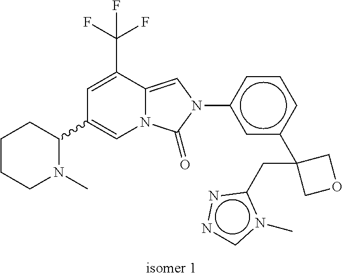 |
| 187 | 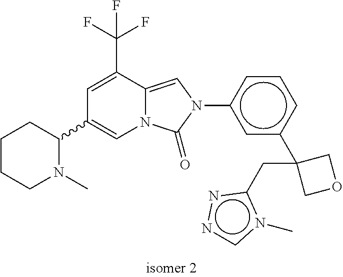 |
| 188 | 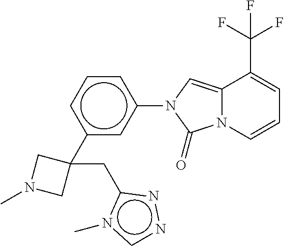 |
| 189 | 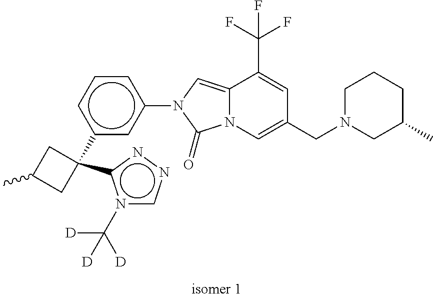 |
| 190 | 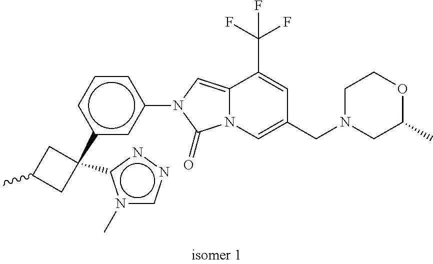 |
| 191 | 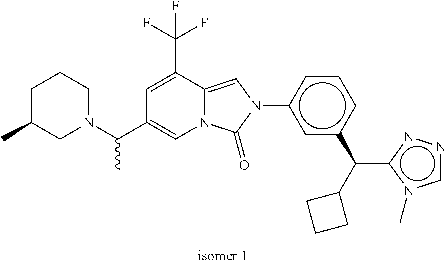 |
| 192 | 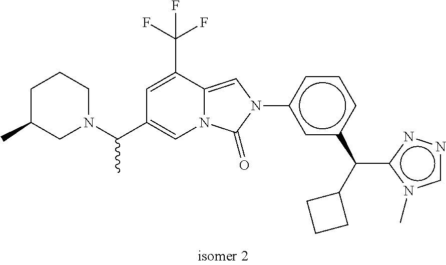 |
| 193 | 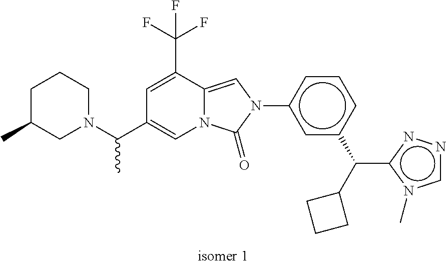 |
| 194 | 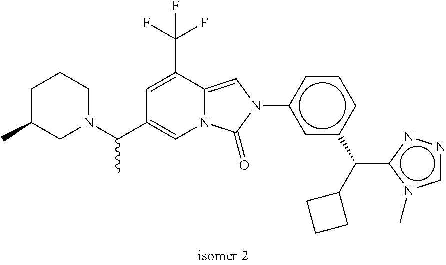 |
| 195 | 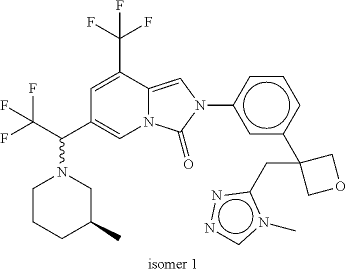 |
| 196 | 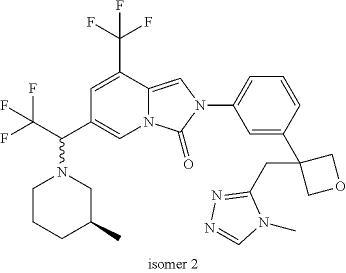 [SMILES: C[C@H]1CCCN(C(c2cc(C(F)(F)F)c3cn(-c4cccc(C5(Cc6nncn6C)COC5)c4)c(=O)n3c2)C(F)(F)F)C1] |
| 197 | 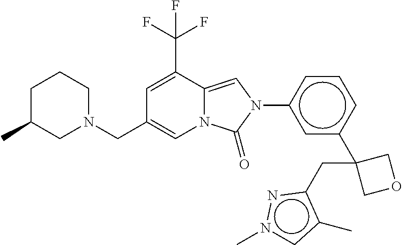 |
| 198 | 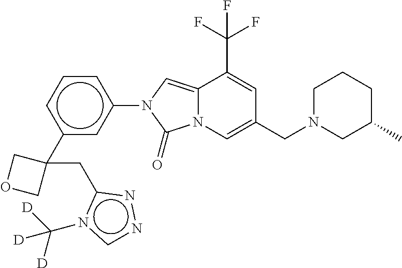 |
| 199 | 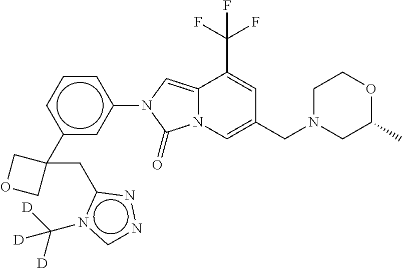 |
| 200 | 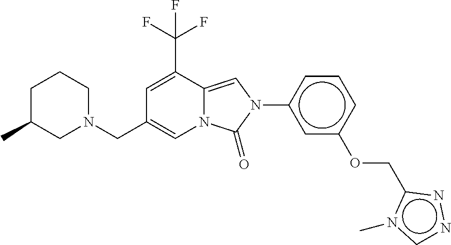 |
| 201 | 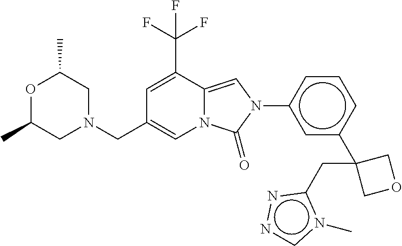 |
| 202 | 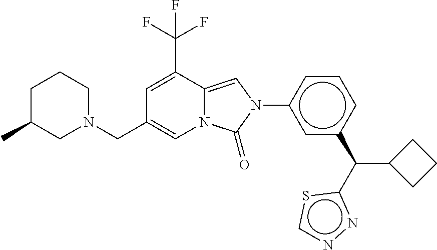 |
| 203 | 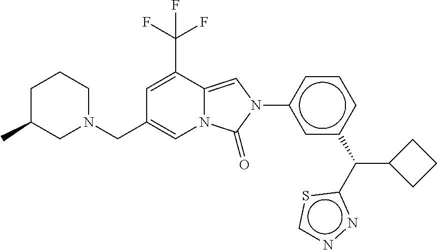 |
| 204 | 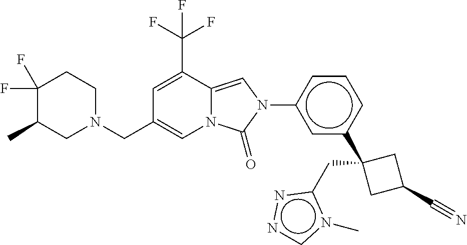 |
| 205 | 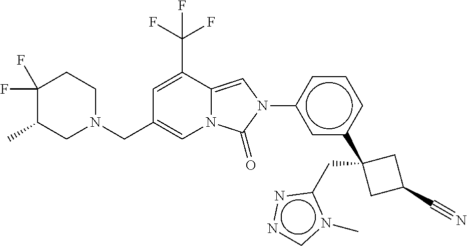 |
| 206 | 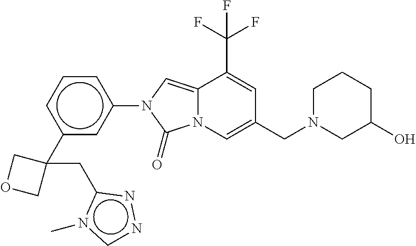 |
| 207 | 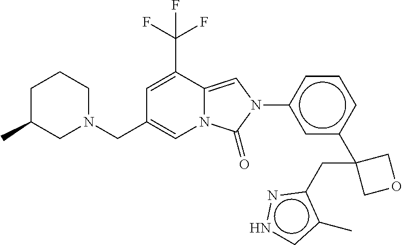 |
| 208 | 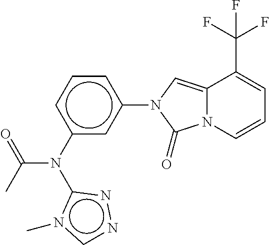 |
| 209 | 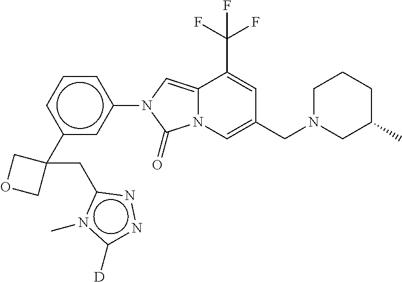 |
| 210 | 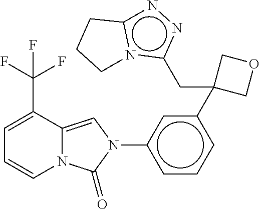 |
| 211 | 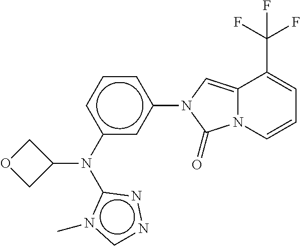 |
| 212 | 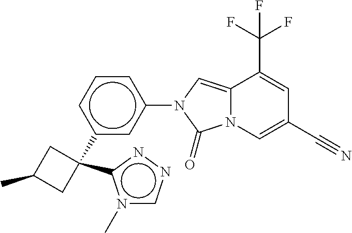 |
| 213 | 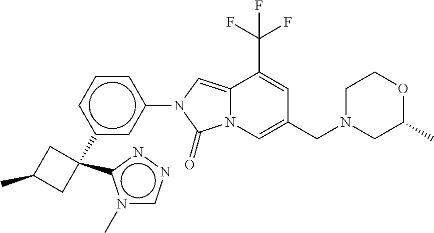 |
| 214 | 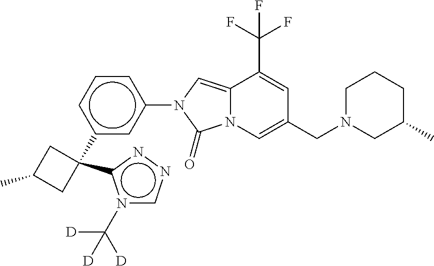 |
| 215 | 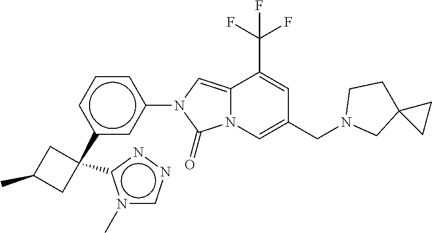 |
| 216 | 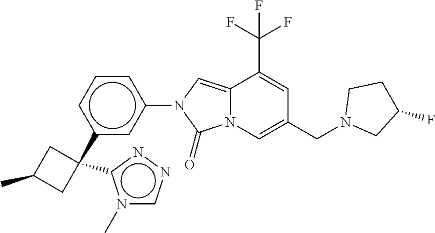 |
| 217 | 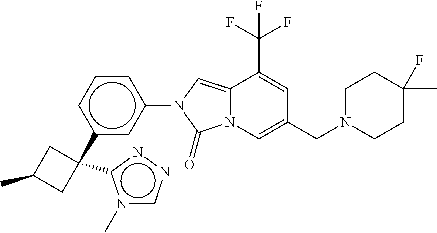 |
| 218 | 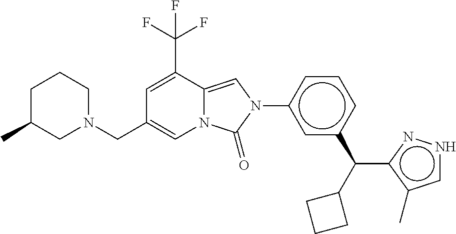 |
| 219 | 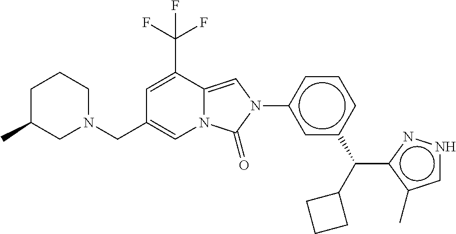 |
| 220 | 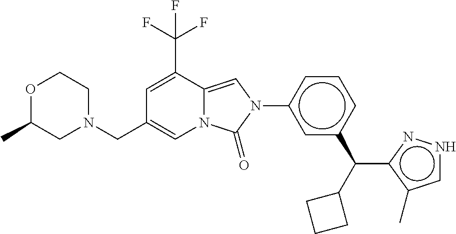 |
| 221 | 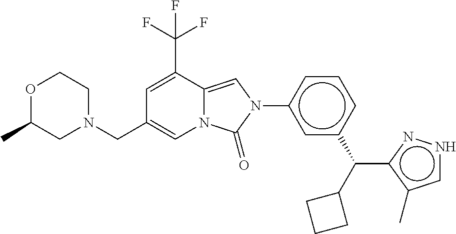 |
| 222 | 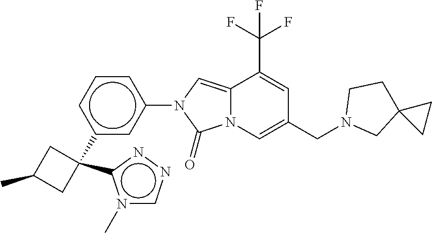 |
| 223 | 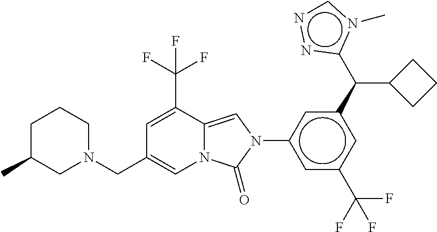 |
| 224 | 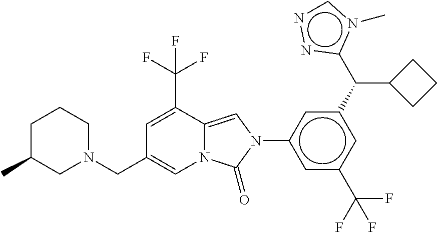 |
| 225 | 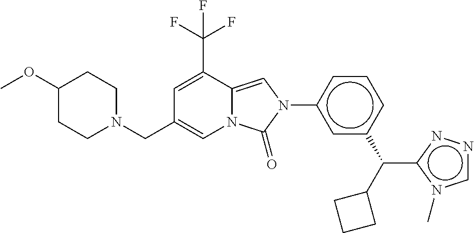 |
| 226 | 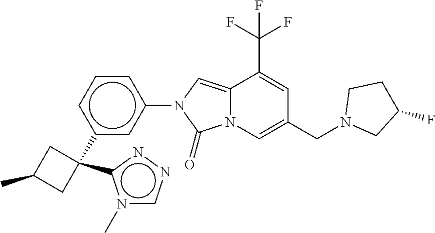 |
| 227 | 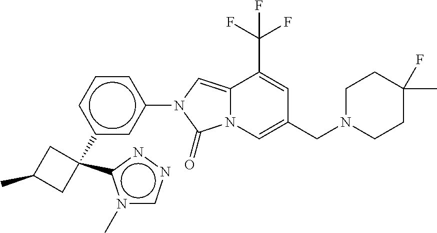 |
| 228 | 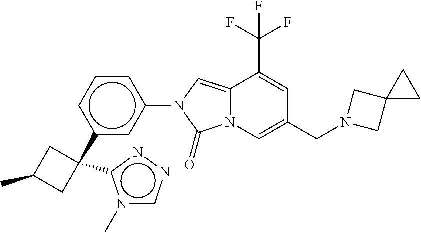 |
| 229 | 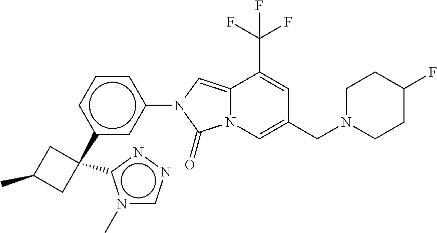 |
| 230 | 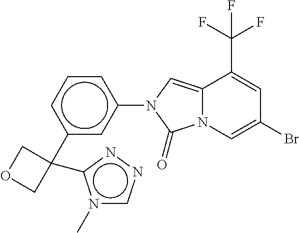 |
| 231 | 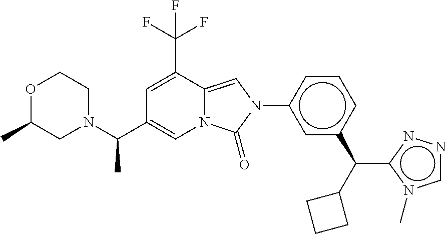 |
| 232 | 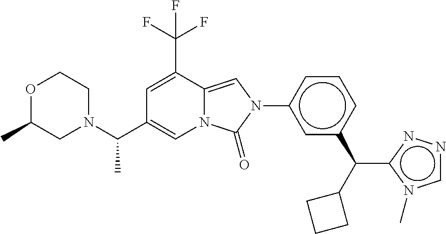 |
| 233 | 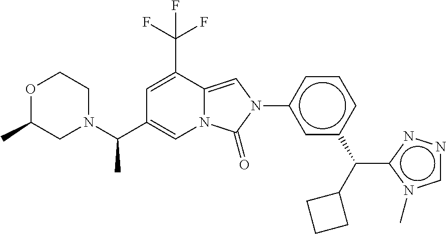 |
| 234 | 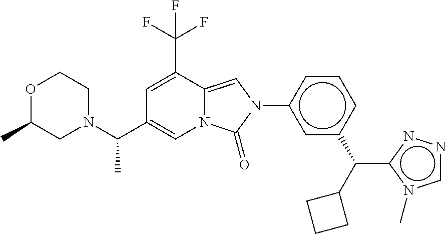 |
| 235 | 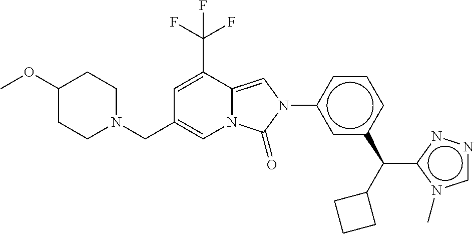 |
| 236 | 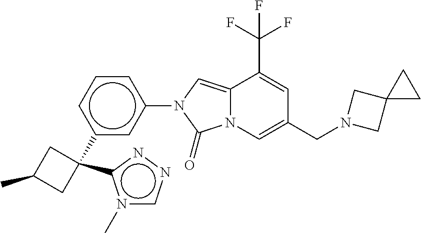 |
| 237 | 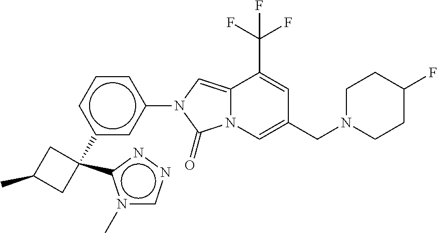 |
| 238 | 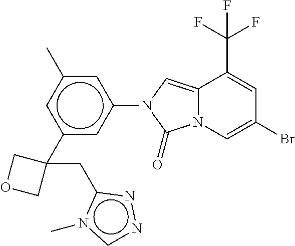 |
| 239 | 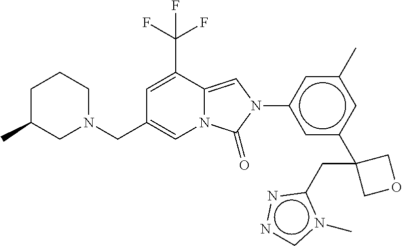 |
| 240 | 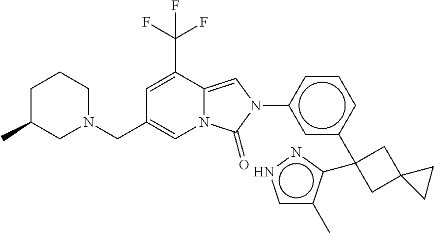 |
| 241 | 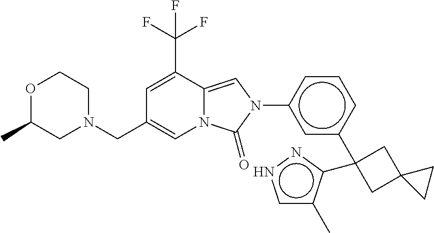 |
| 242 | 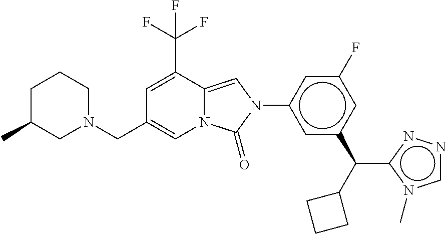 |
| 243 | 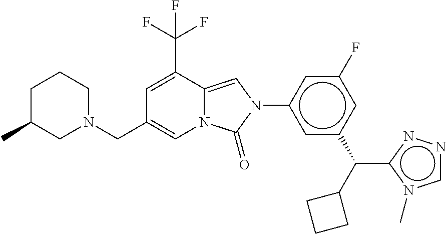 |
| 244 | 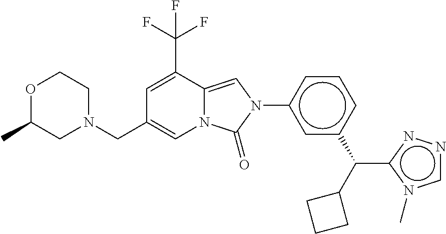 |
| 245 | 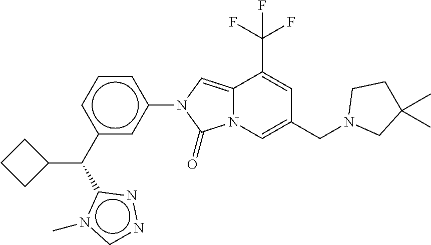 |
| 246 | 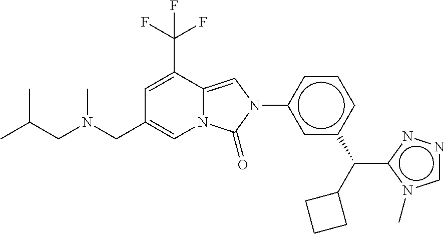 |
| 247 | 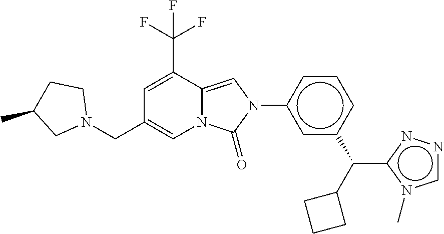 |
| 248 | 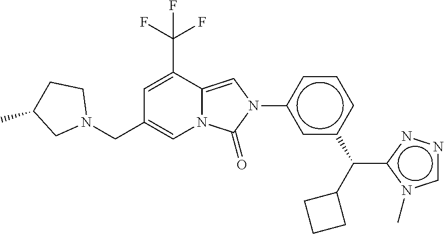 |
| 249 | 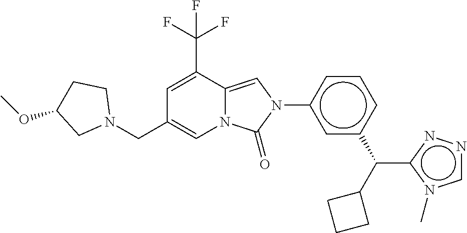 |
| 250 | 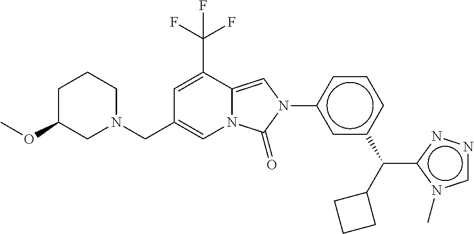 |
| 251 | 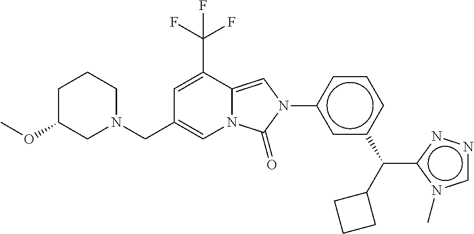 |
| 252 | 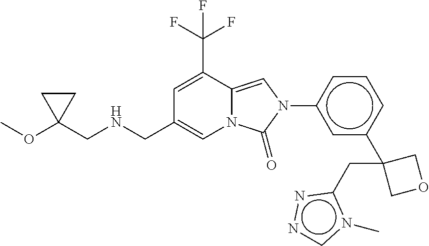 |
| 253 | 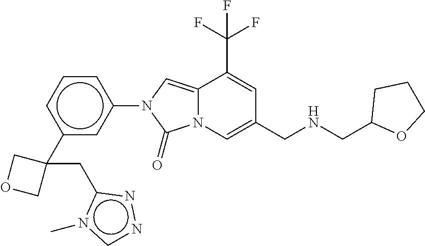 |
| 254 | 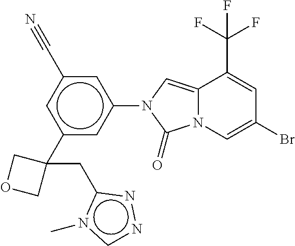 |
| 255 | 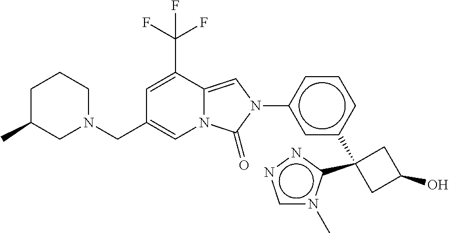 |
| 256 | 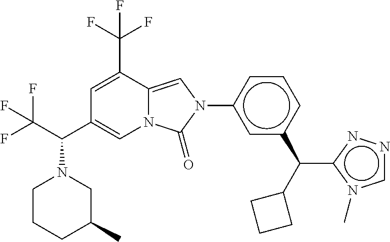 |
| 257 | 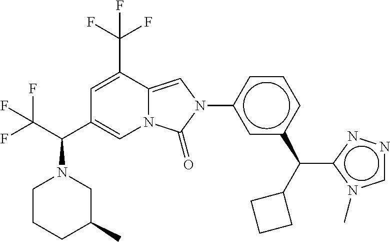 |
| 258 | 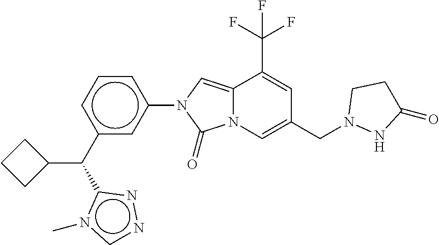 |
| 259 | 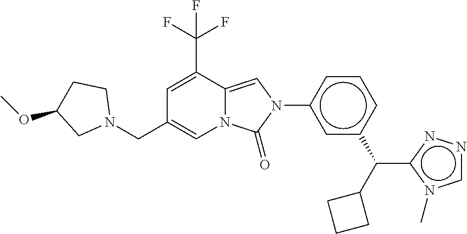 |
| 260 | 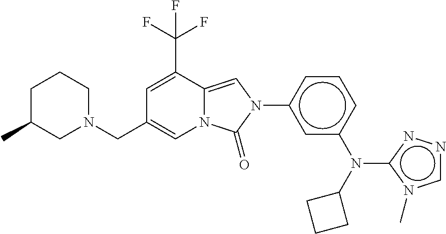 |
| 261 | 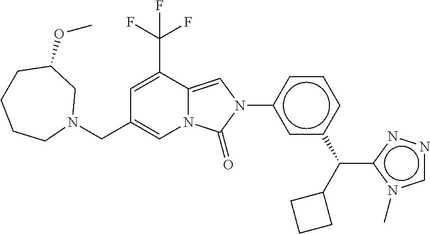 |
| 262 | 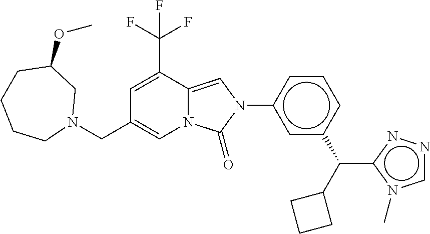 |
| 263 | 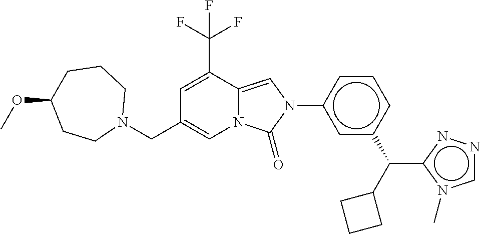 |
| 264 | 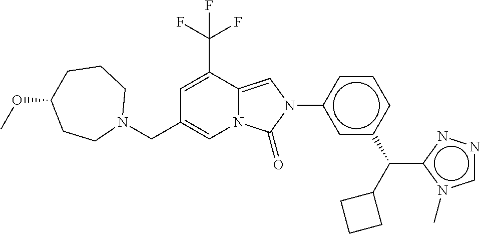 |
| 265 | 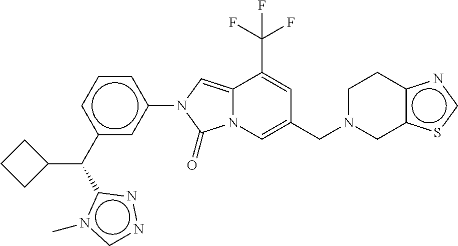 |
| 266 | 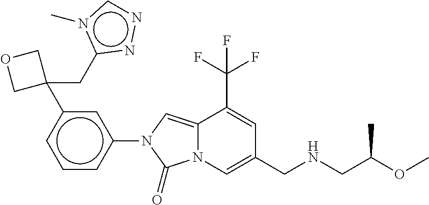 |
| 267 | 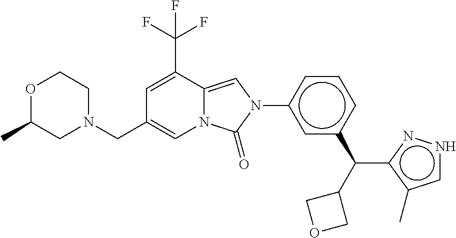 |
| 268 | 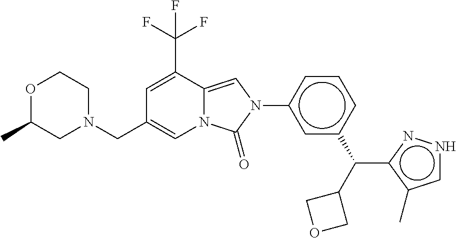 |
| 269 | 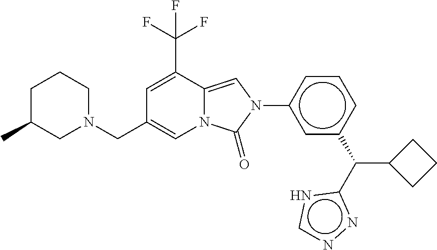 |
| 270 | 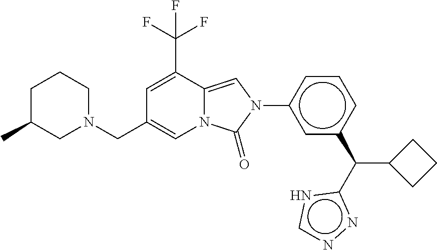 |
| 271 | 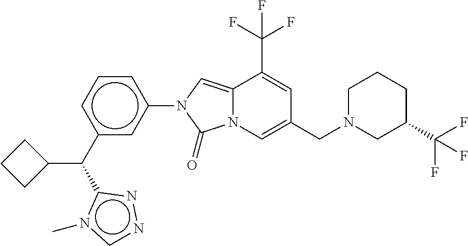 |
| 272 | 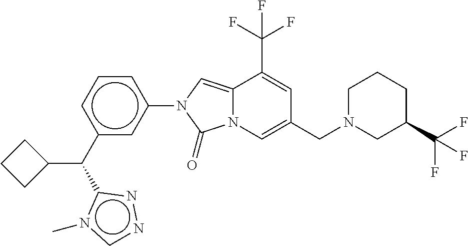 |
| 273 | 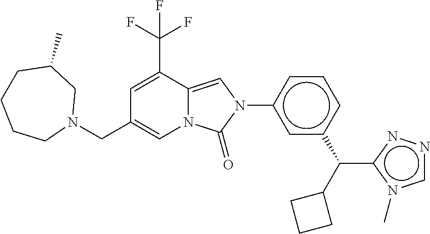 |
| 274 | 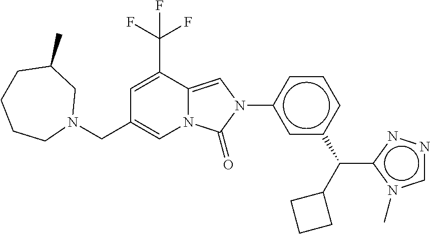 |
| 275 | 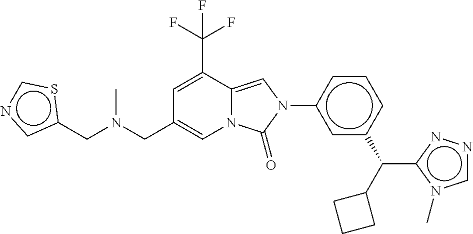 |
| 276 | 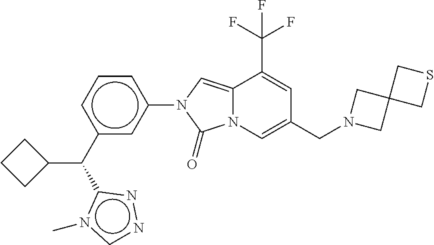 |
| 277 | 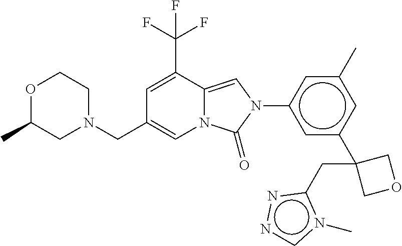 |
| 278_p1 | 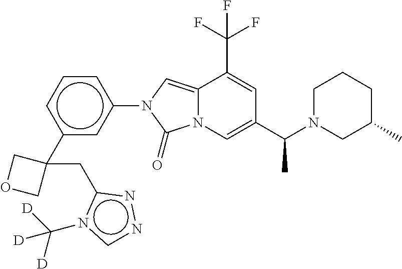 |
| 278_p2 | 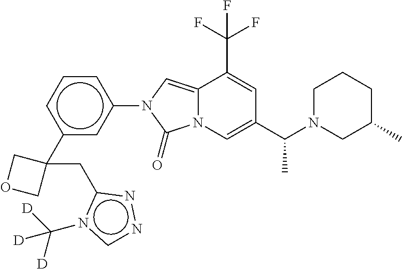 |
| 279 | 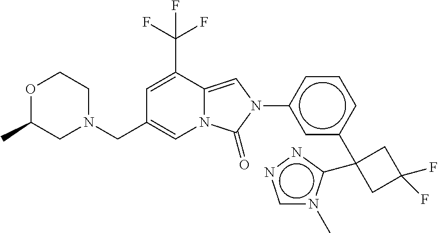 |
| 280 | 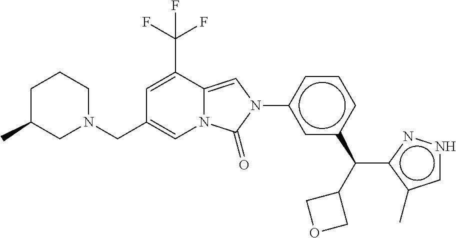 |
| 281 | 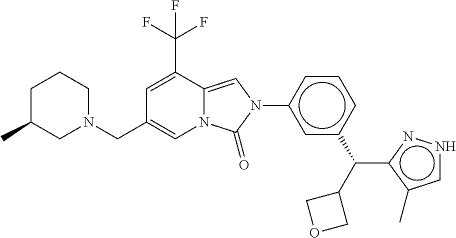 |
| 282 | 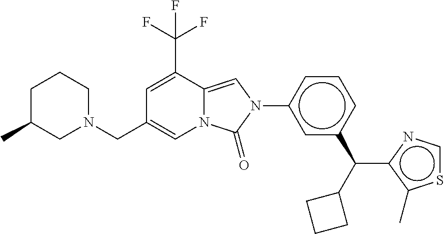 |
| 283 | 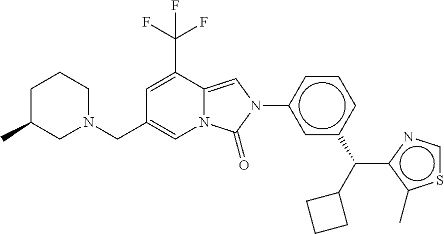 |
| 284 | 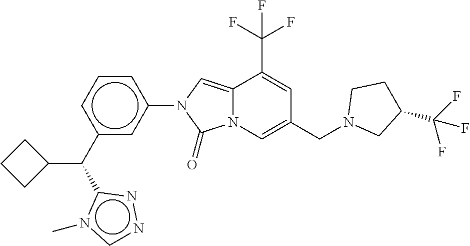 |
| 285 | 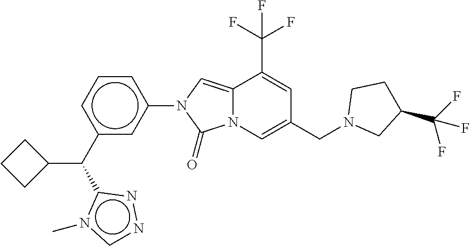 |
| 286 | 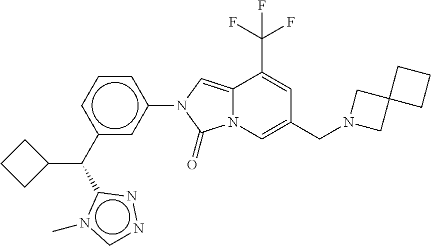 |
| 287_P1 | 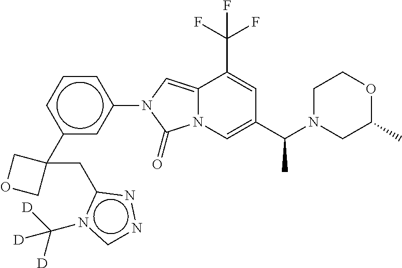 |
| 287_P2 | 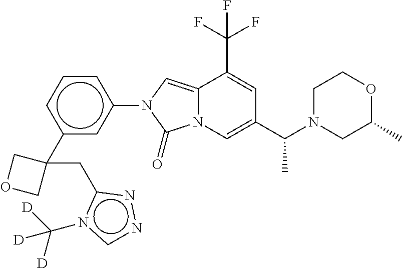 |
| 288 | 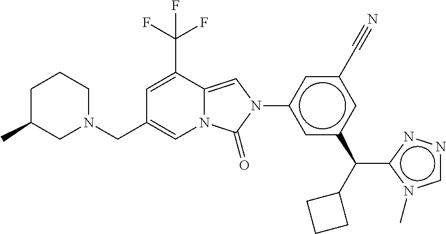 |
| 289 | 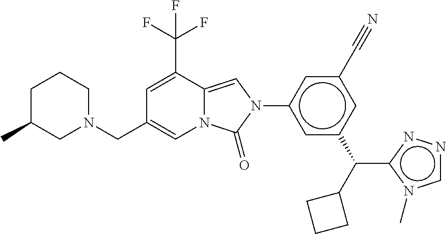 |
| 290 | 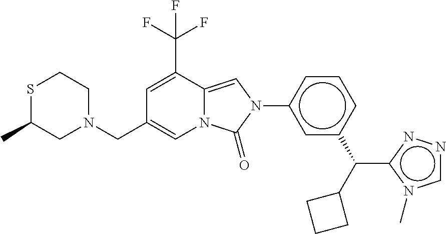 |
| 291 | 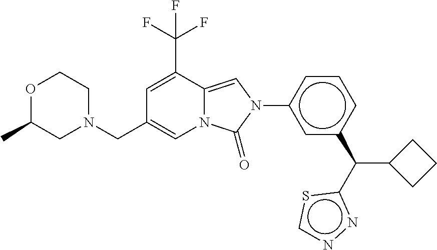 |
| 292 | 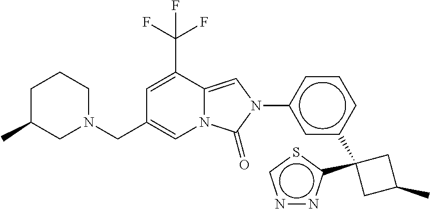 |
| 293 | 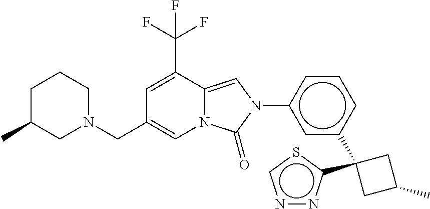 |
| 294 | 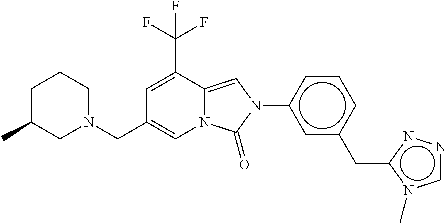 |
| 295 | 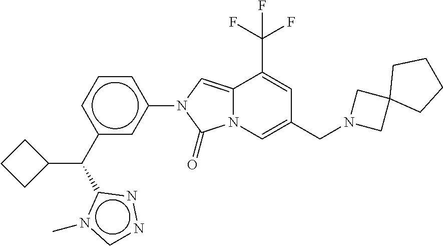 |
| 296 | 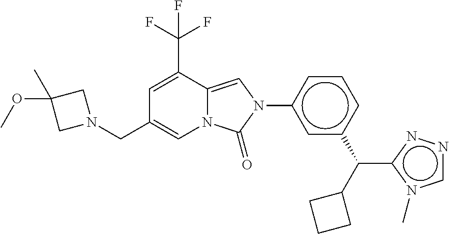 |
| 297 | 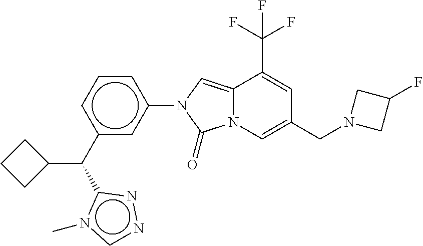 |
| 298 | 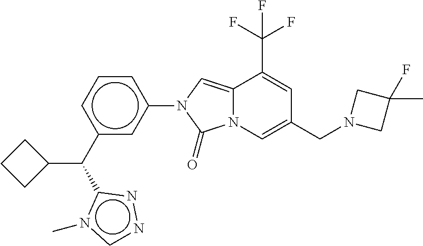 |
| 299 | 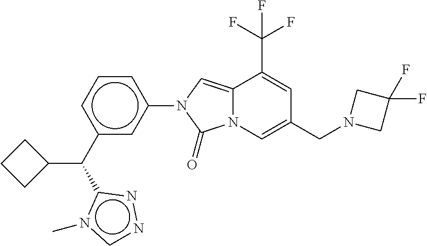 |
| 300 | 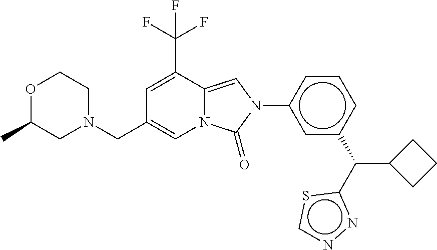 |
| 301 | 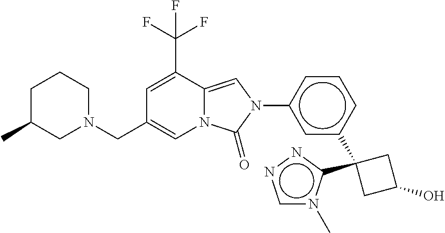 |
| 302 | 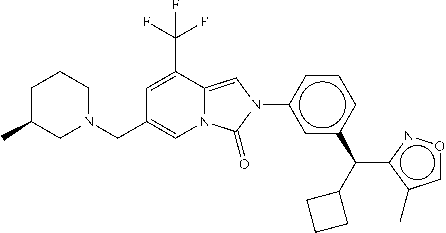 |
| 303 | 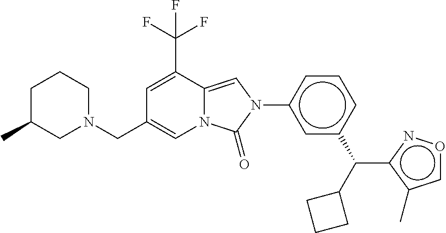 |
| 304 | 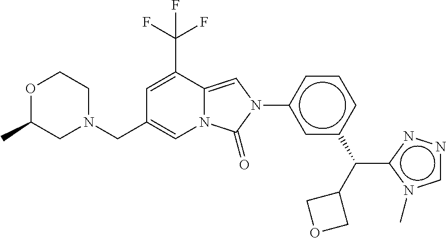 |
| 305 | 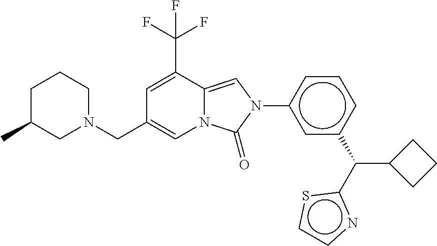 |
| 306 | 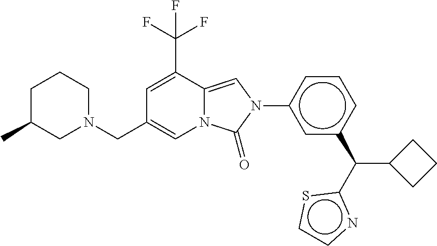 |
| 307 | 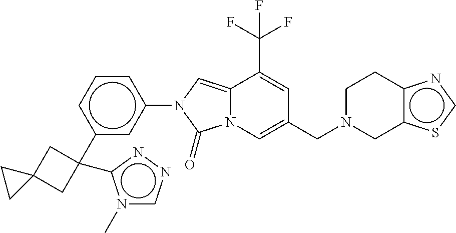 |
| 308 | 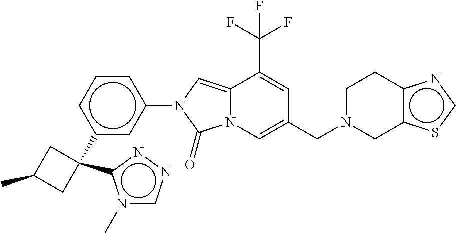 |
| 309 | 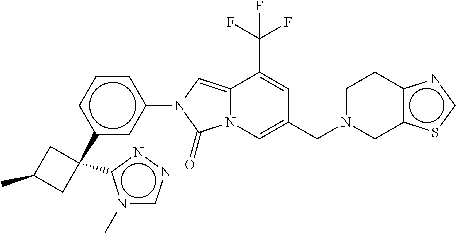 |
| 310 | 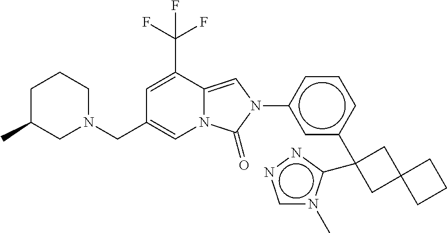 |
| 311 | 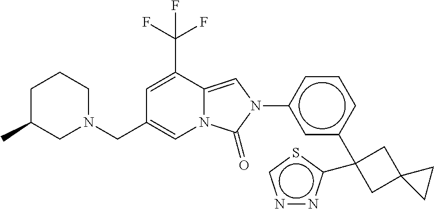 |
| 312 | 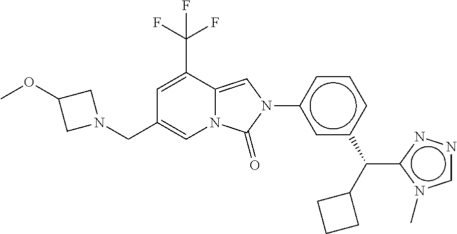 |
| 313 | 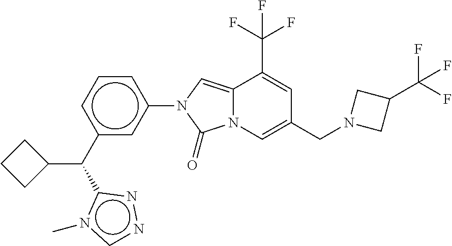 |
| 314 | 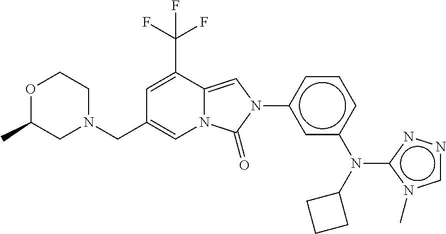 |
| 315 | 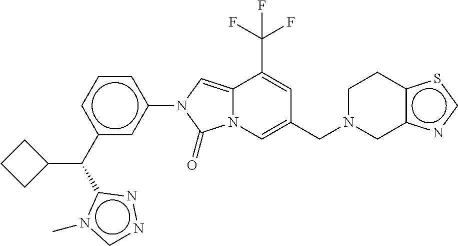 |
| 316 | 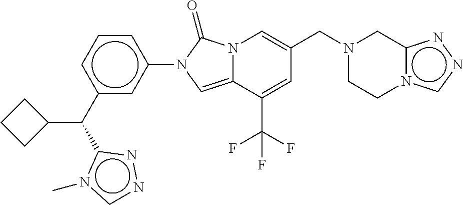 |
| 317 | 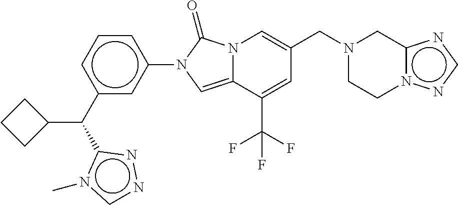 |
| 318 | 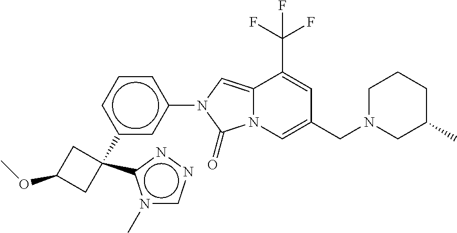 |
| 319 | 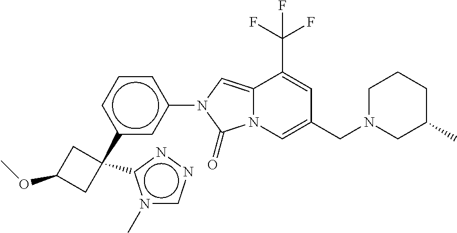 |
| 329 | 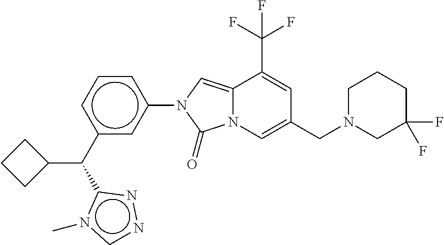 |
| 321 | 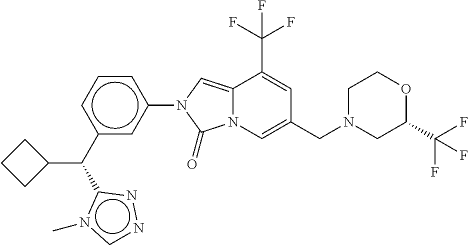 |
| 322 | 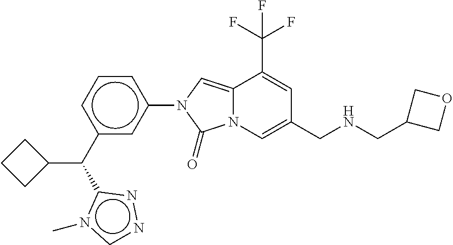 |
| 323 | 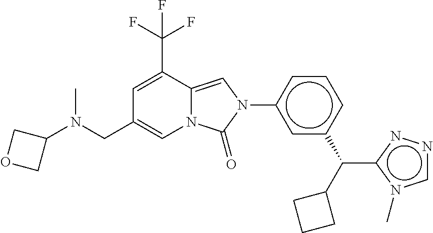 |
| 324 | 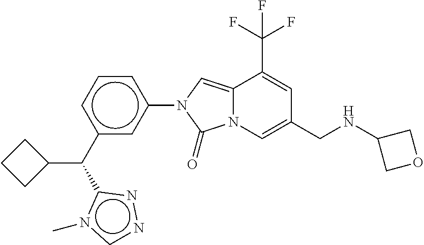 |
| 325 | 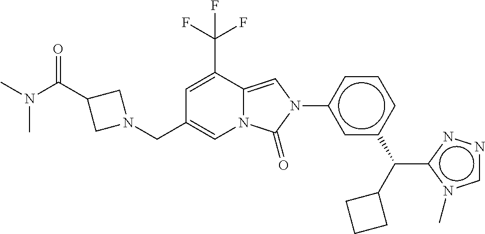 |
| 326 | 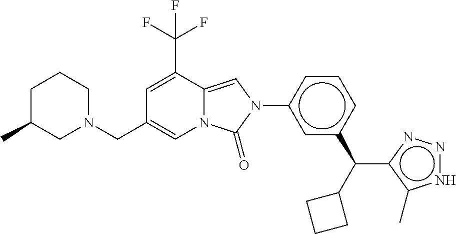 |
| 327 | 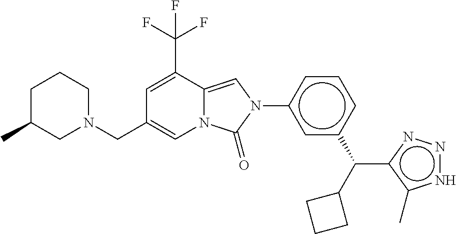 |
| 328 | 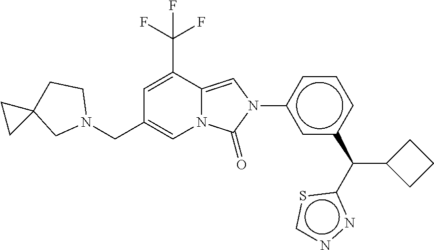 |
| 329 | 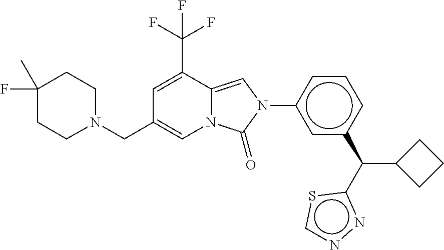 |
| 330 | 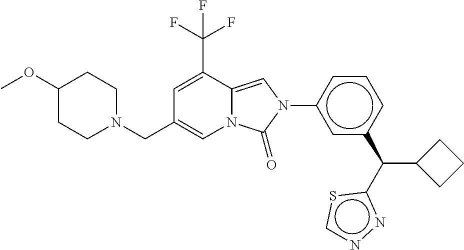 |
| 331 | 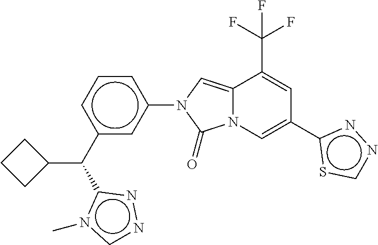 |
| 332_p1 | 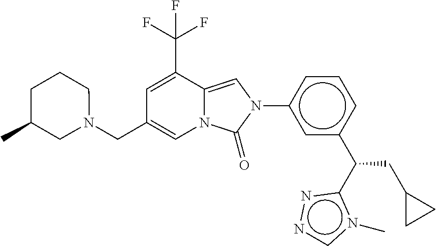 |
| 332-p2 | 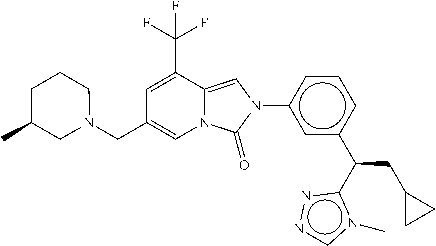 |
| 333 | 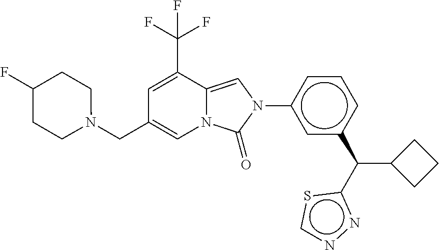 |
| 334 | 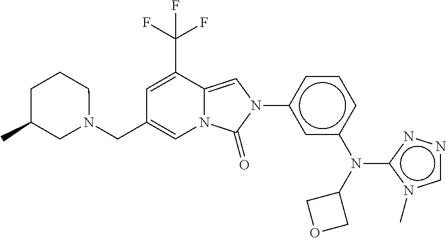 |
| 335 | 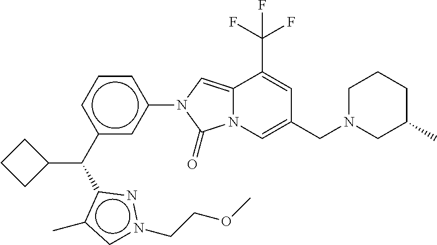 |
| 336 | 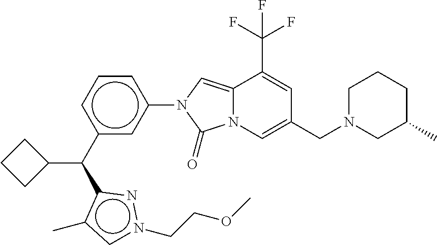 |
| 337 | 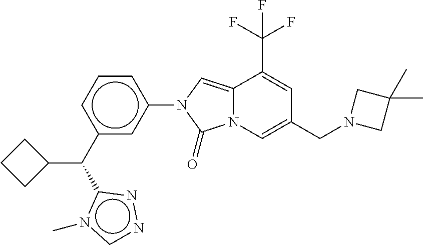 |
| 338 | 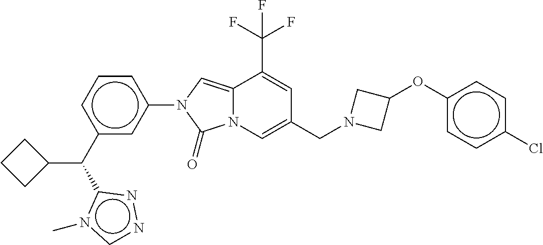 |
| 339_P1 | 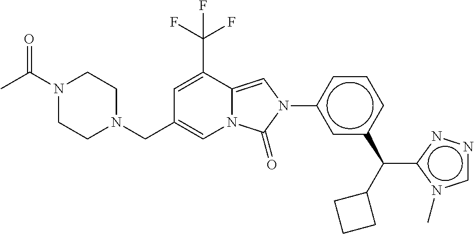 |
| 339_P2 | 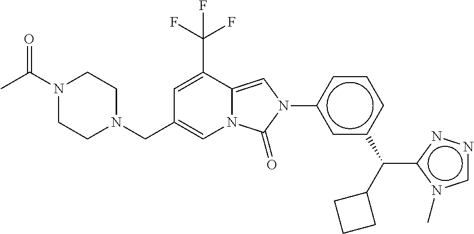 |
| 340_P1 | 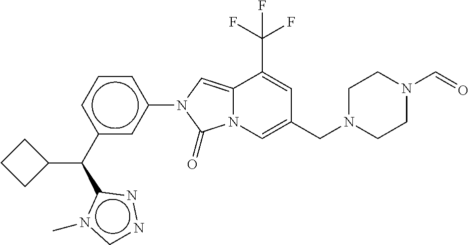 |
| 340_P2 | 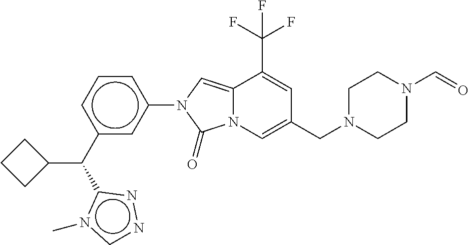 |
| 341 | 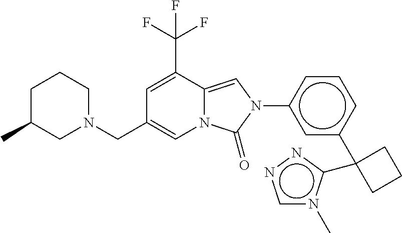 |
| 342 | 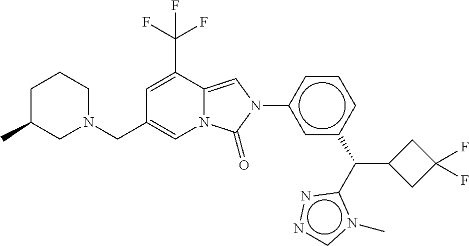 |
| 343 | 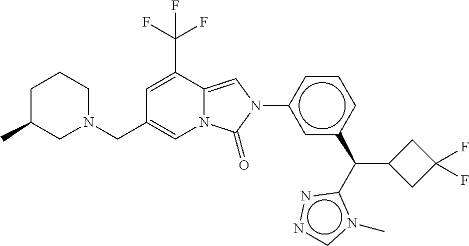 |
| 344 | 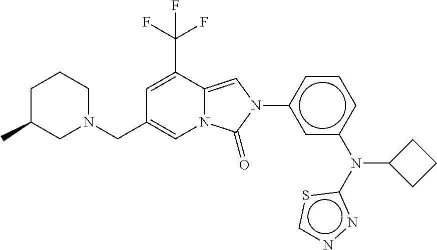 |
| 345 | 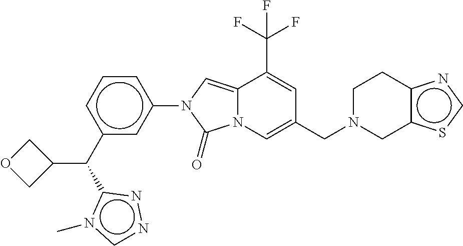 |
| 346 | 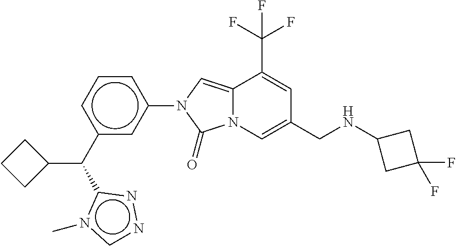 |
| 347 | 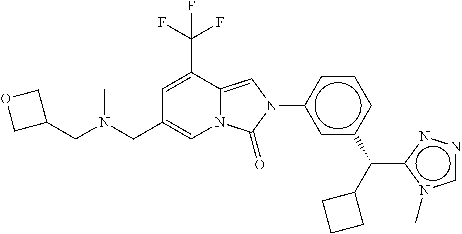 |
| 348 | 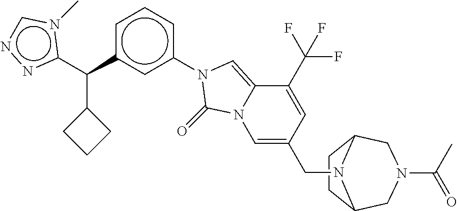 |
| 349 | 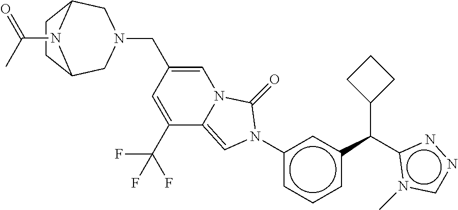 |
| 350 | 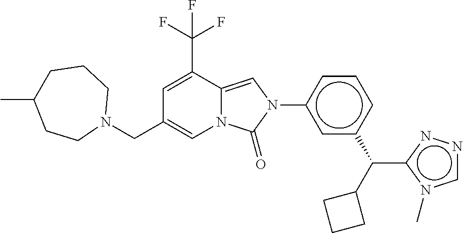 |
| 351_P1 | 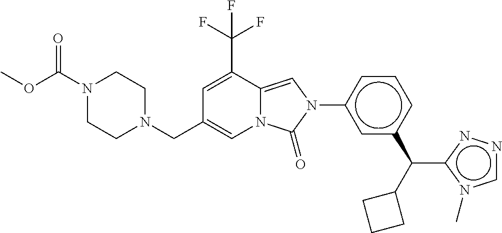 |
| 351_P2 | 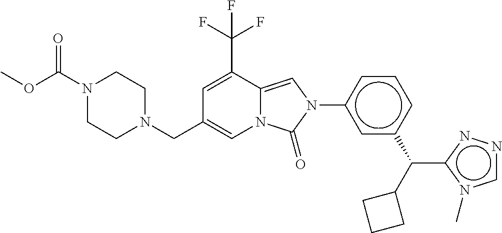 |
| 352_P1 | 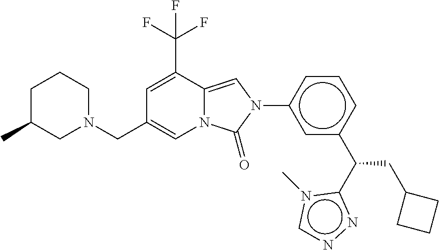 |
| 352_P2 | 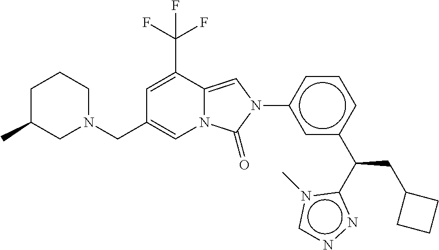 |
| 353 | 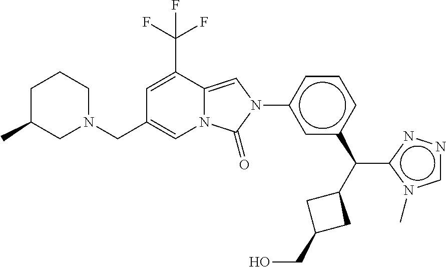 |
| 354 | 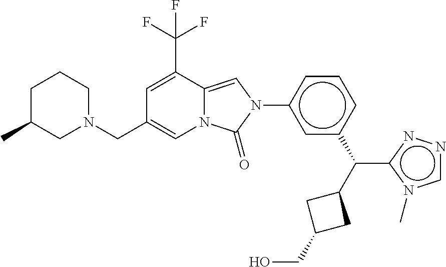 |
| 355 | 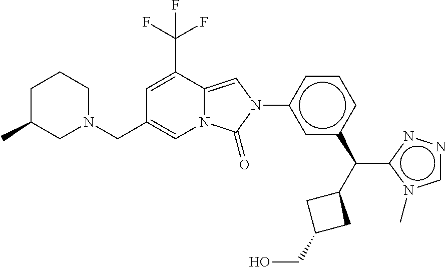 |
| 356 | 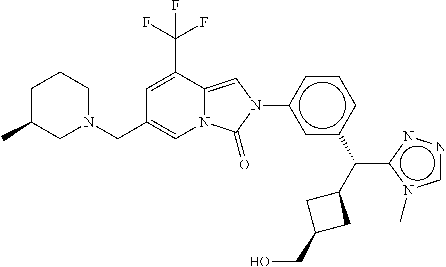 |
| 357 | 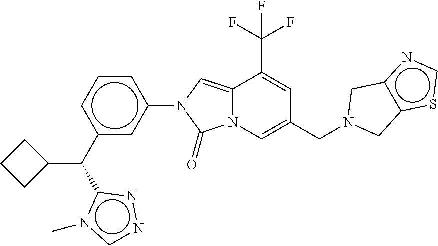 |
| 358 | 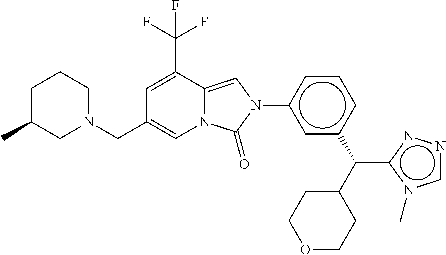 |
| 359 | 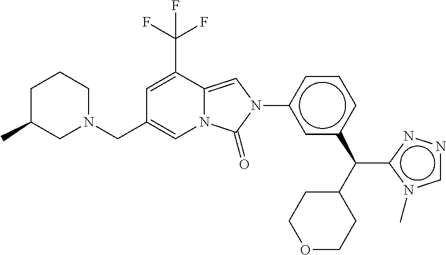 |
| 360 | 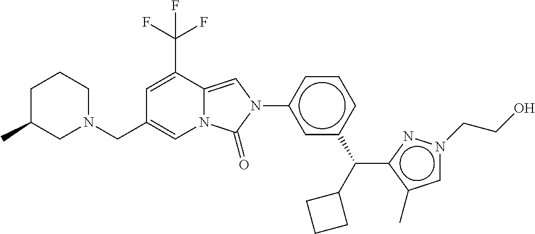 |
| 361 | 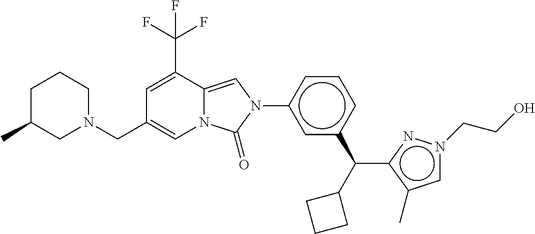 |
| 362_P1 | 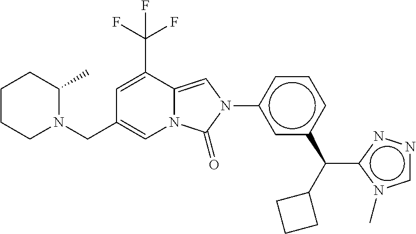 |
| 362_P2 | 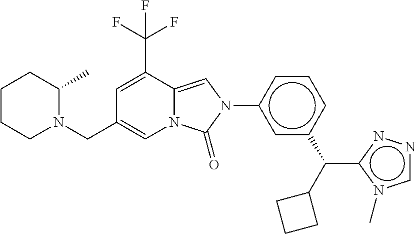 |
| 363_P1 | 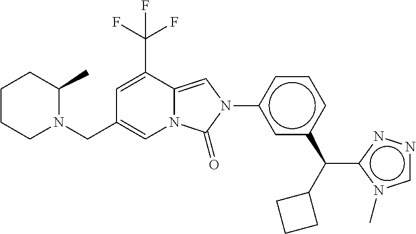 |
| 363_P2 | 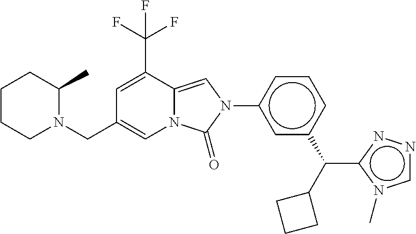 |
| 364_P1 | 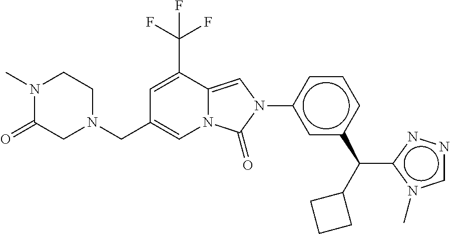 |
| 364_P2 | 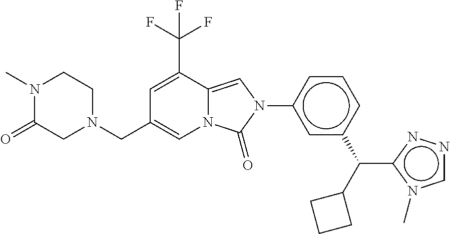 |
| 365 | 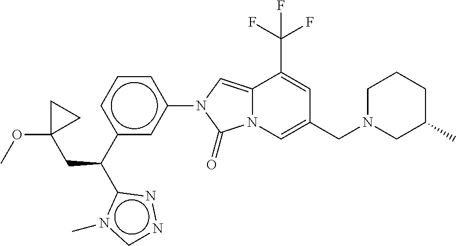 |
| 366 | 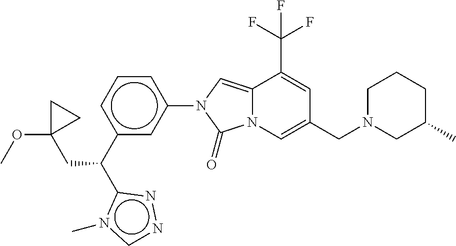 |
| 367 | 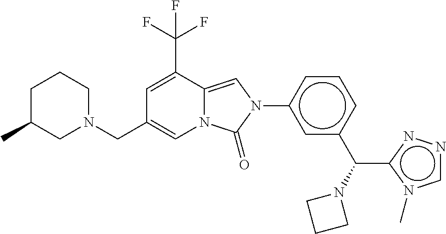 |
| 368 | 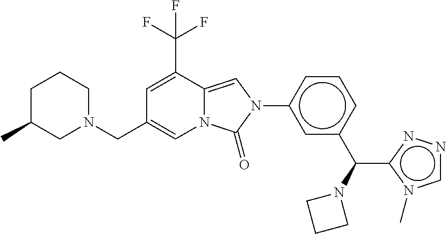 |
| 369 | 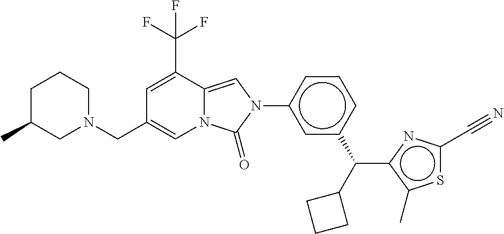 |
| 370 | 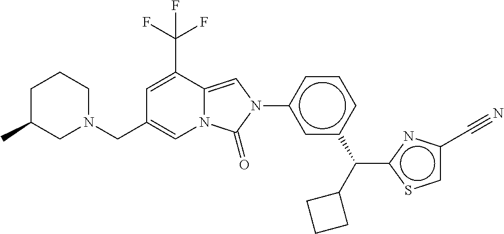 |
| 371 | 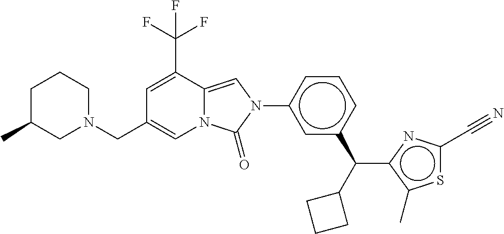 |
| 372 | 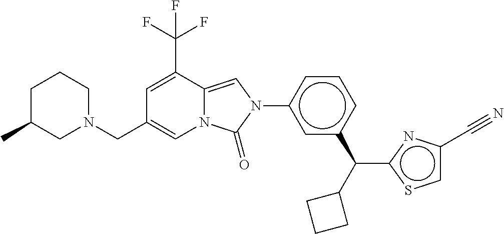 |
| 373 | 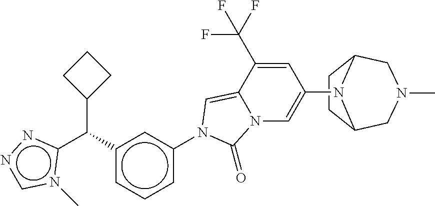 |
| 374 | 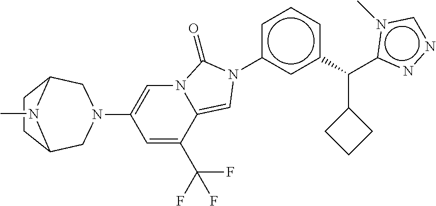 |
| 375 | 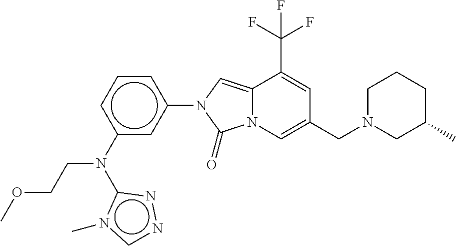 |
| 376 | 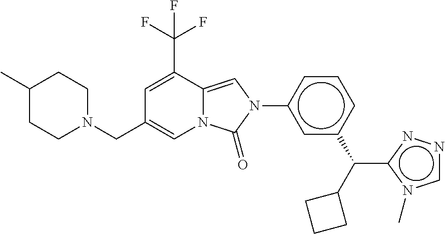 |
| 377 | 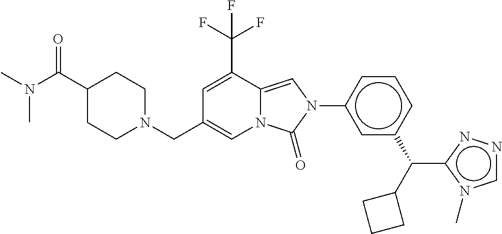 |
| 378 | 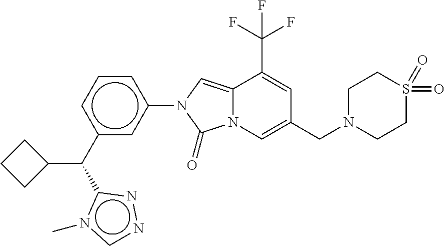 |
| 379 | 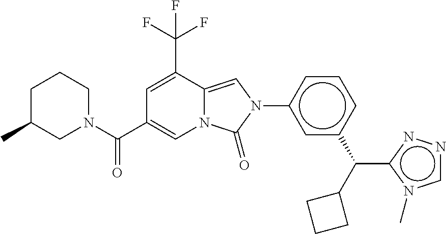 |
| 380_P1 | 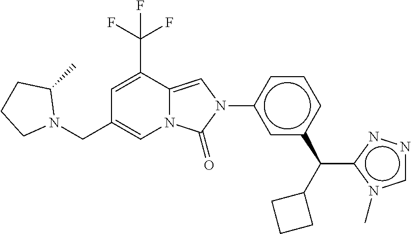 |
| 380_P2 | 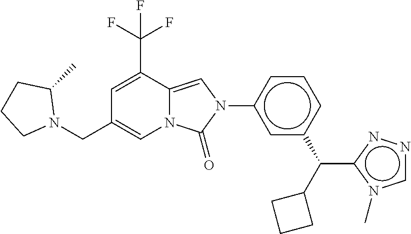 |
| 381_P1 | 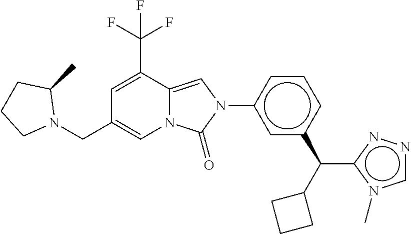 |
| 381_P2 | 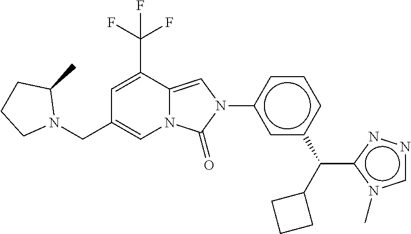 |
| | 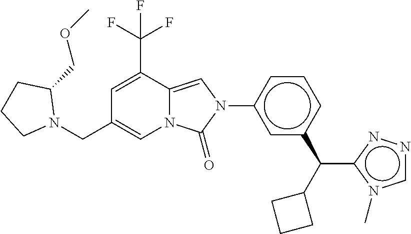 |
| | 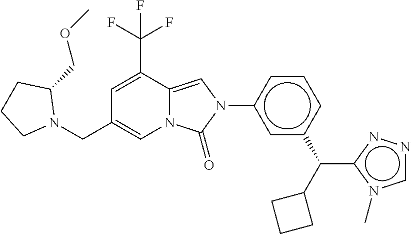 |
| 382_P1 | 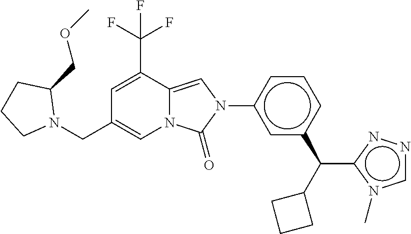 |
| 382_P2 | 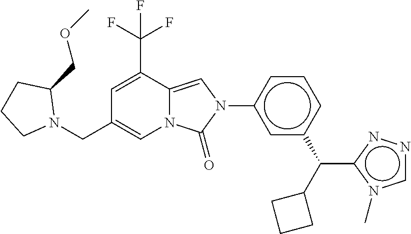 |
| 383 | 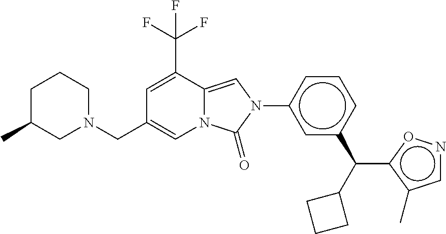 |
| 384 | 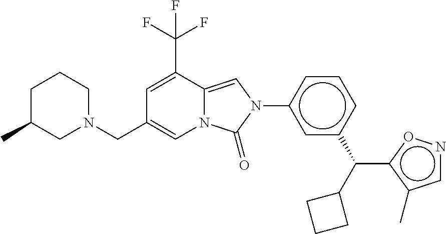 |
| 385 | 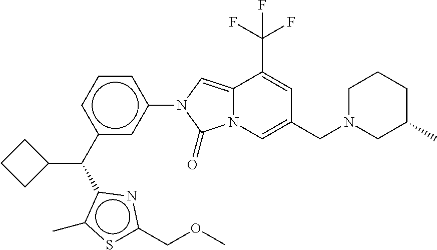 |
| 386 | 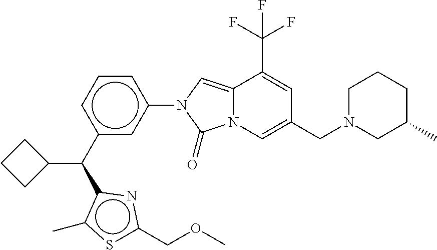 |
| 387 | 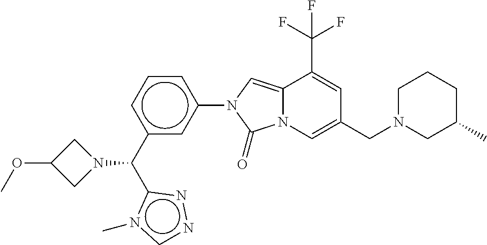 |
| 388 | 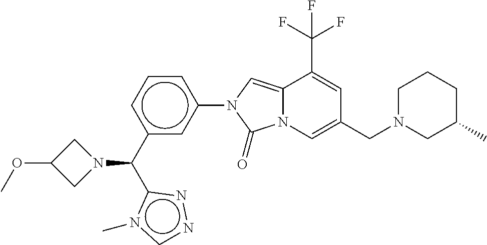 |
| 389 | 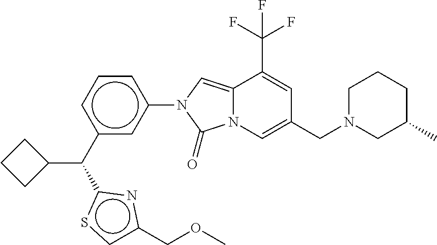 |
| 390 | 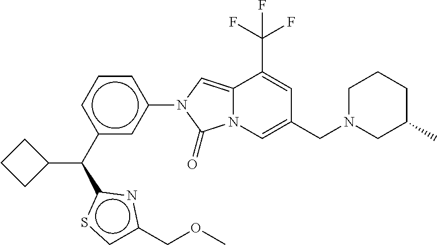 |
| 391 | 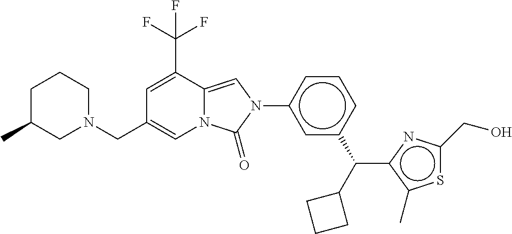 |
| 392 | 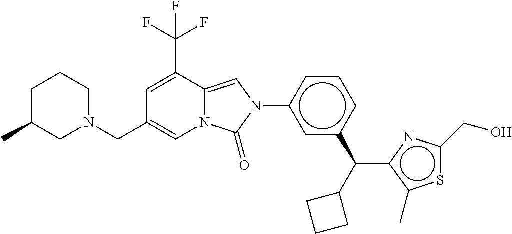 |
| 393 | 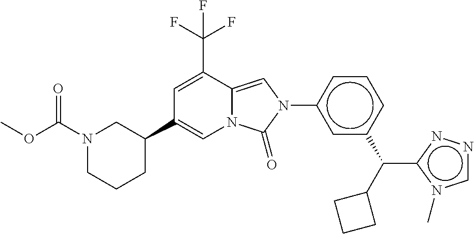 |
| 394 | 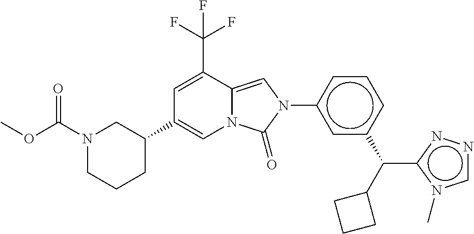 |
| 395 | 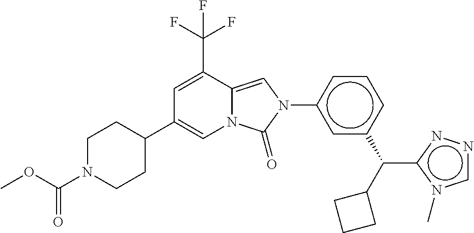 |
| 396 | 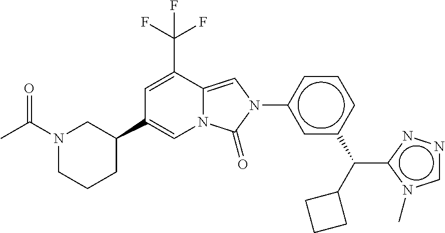 |
| 397 | 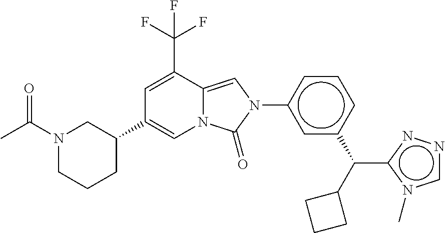 |
| 398 | 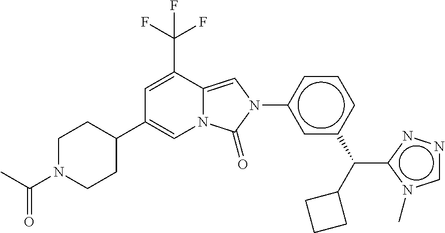 |
| 399 | 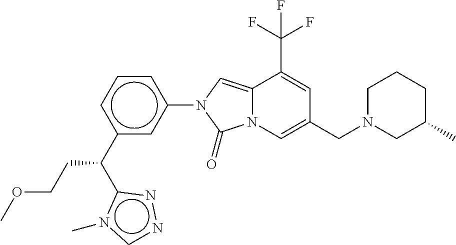 |
| 400 | 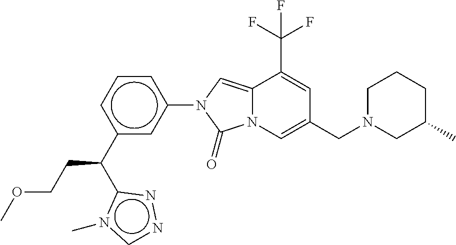 |
| 401_P1 | 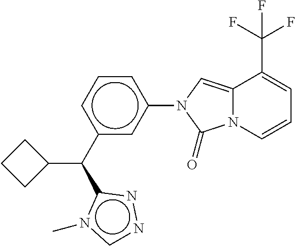 |
| 401_P2 | 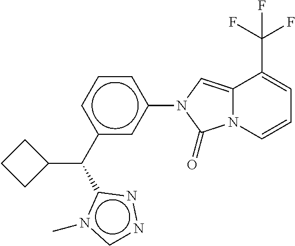 |
| 402 | 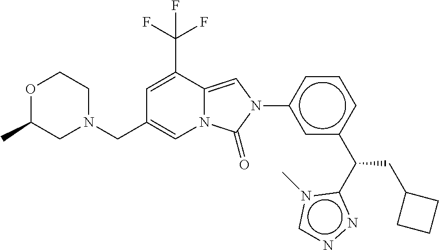 |
| 403_P1 | 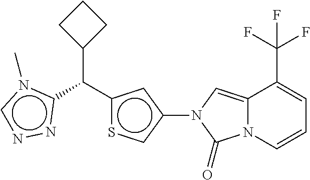 |
| 403_P2 | 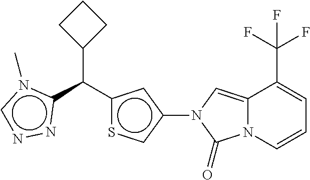 |
| 404 | 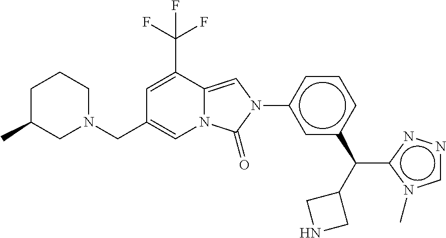 |
| 403 | 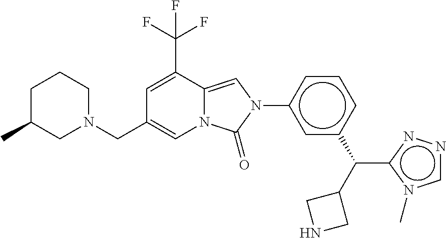 |
| 404 | 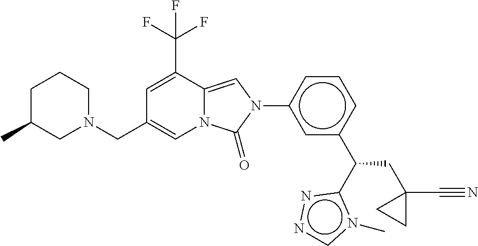 |
| 405 | 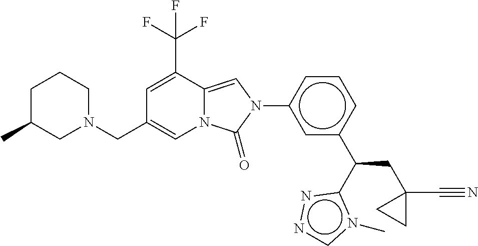 |
| 406 | 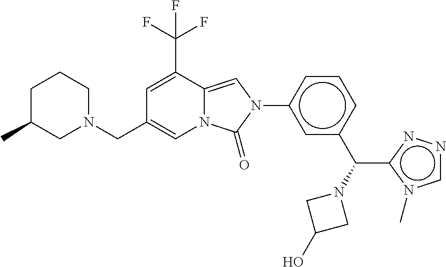 |
| 407 | 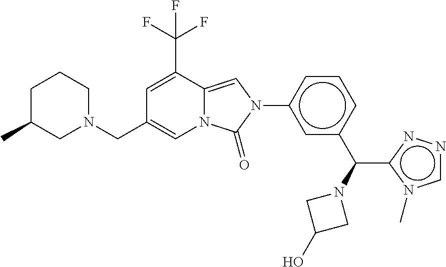 |
| 408 | 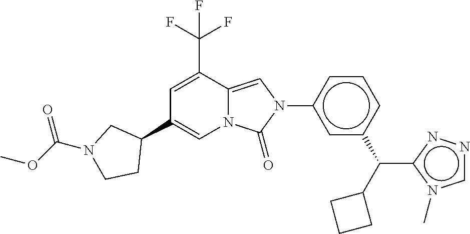 |
| 409 | 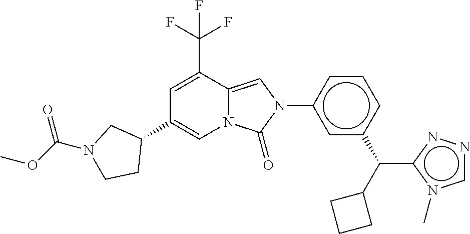 |
| 410 | 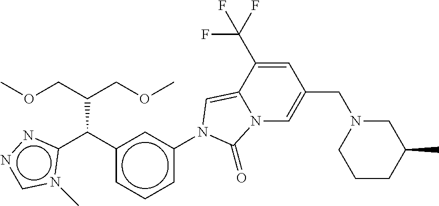 |
| 411 | 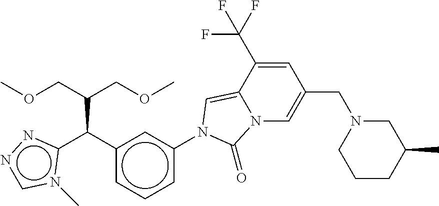 |
| 412 | 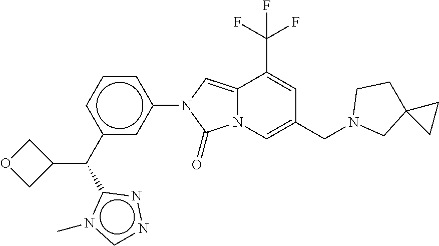 |
| 413 | 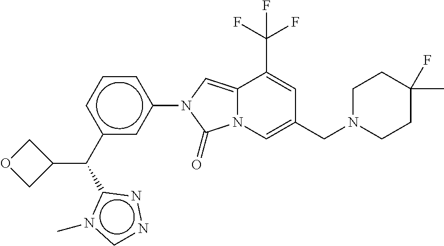 |
| 414 | 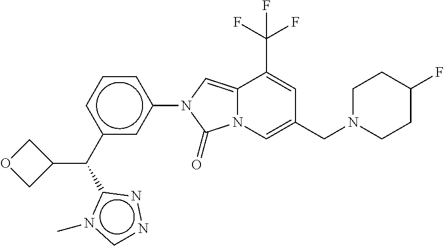 |
| 415 | 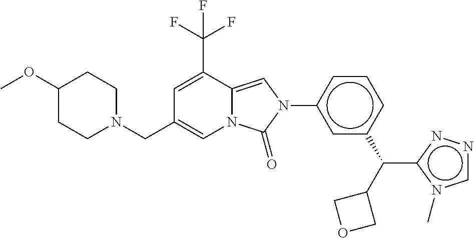 |
| 416 | 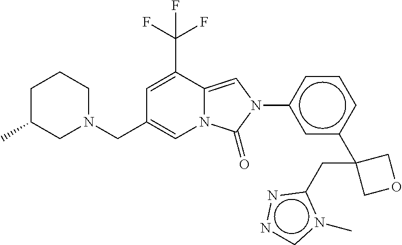 |
| 417 | 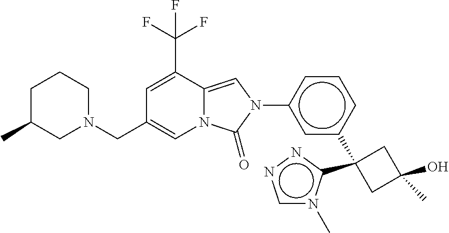 |
| 418 | 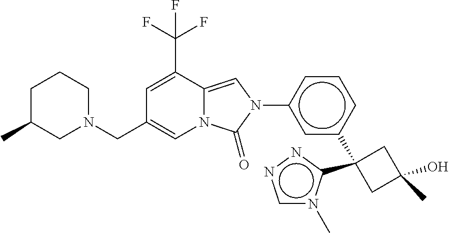 |
| 419 | 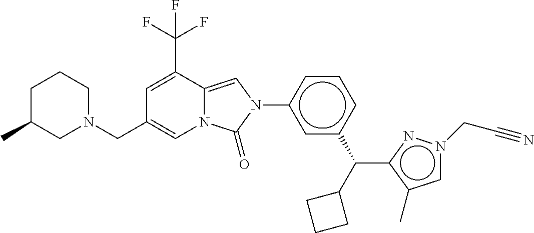 |
| 420 | 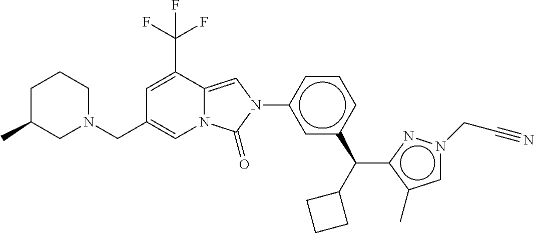 |
| 421 | 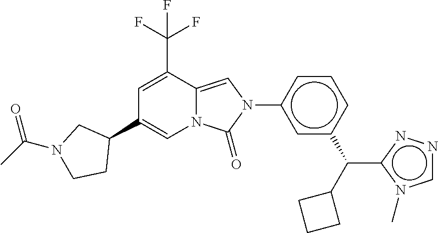 |
| 422 | 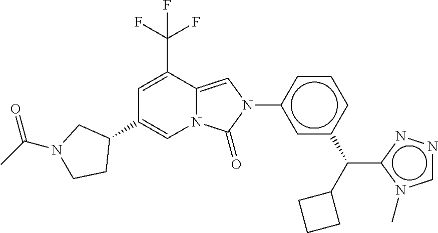 |
| 423 | 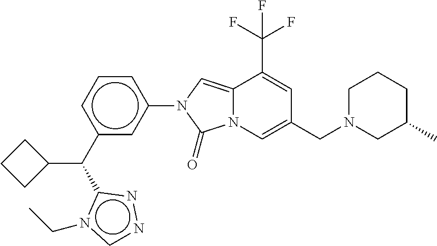 |
| 424 | 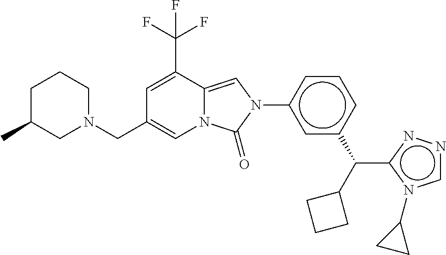 |
| 425 | 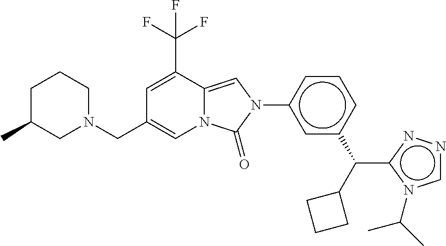 |
| 426 | 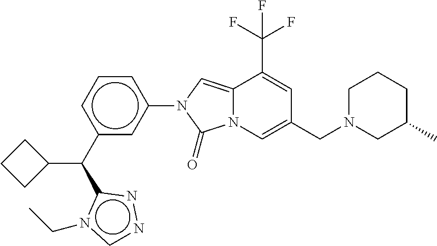 |
| 427 | 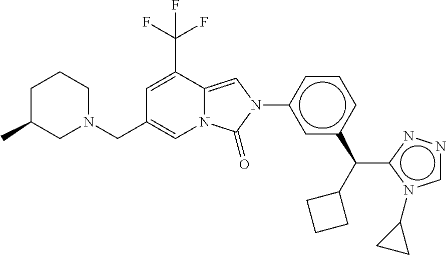 |
| 428 | 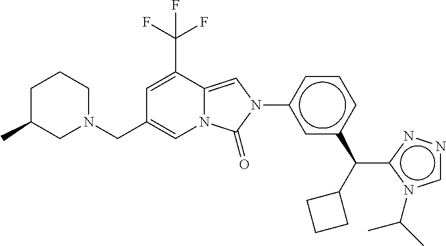 |
| 429 | 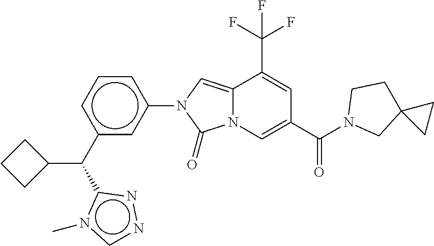 |
| 430 | 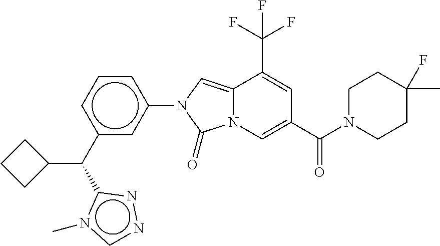 |
| 431 | 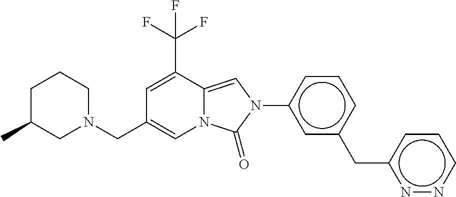 |
| 432 | 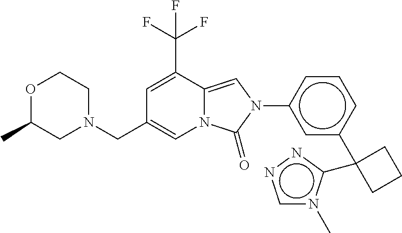 |
| 433 | 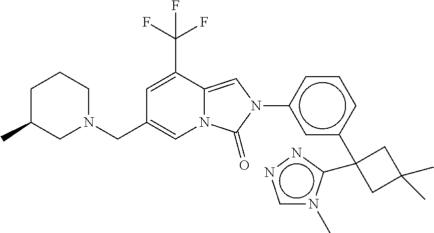 |
| 434_P1 | 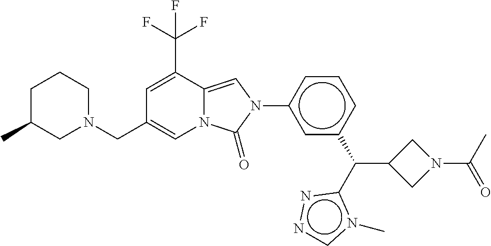 |
| 434_P2 | 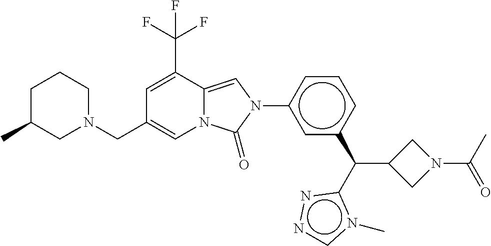 |
| 435 | 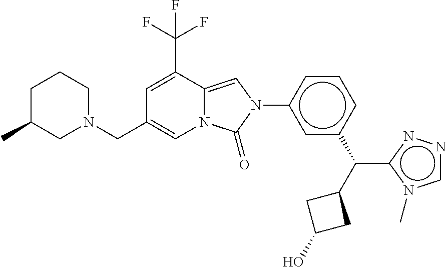 |
| 436 | 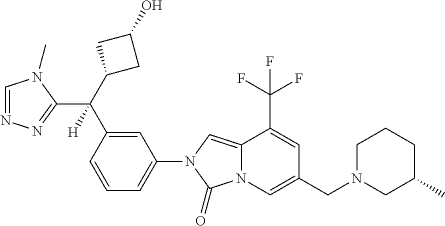 [SMILES: C[C@H]1CCCN(Cc2cc(C(F)(F)F)c3cn(-c4cccc([C@@H](c5nncn5C)[C@H]5C[C@@H](O)C5)c4)c(=O)n3c2)C1] |
| 437 | 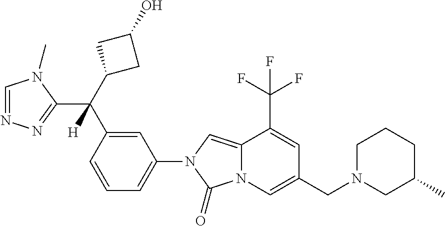 [SMILES: C[C@H]1CCCN(Cc2cc(C(F)(F)F)c3cn(-c4cccc([C@H](c5nncn5C)[C@H]5C[C@@H](O)C5)c4)c(=O)n3c2)C1] |
| 438 | 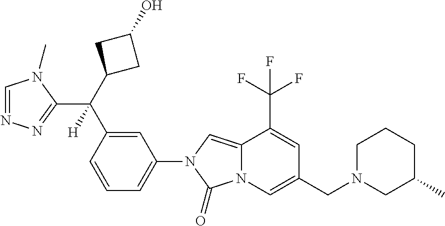 [SMILES: C[C@H]1CCCN(Cc2cc(C(F)(F)F)c3cn(-c4cccc([C@@H](c5nncn5C)[C@H]5C[C@H](O)C5)c4)c(=O)n3c2)C1] |
| 439 | 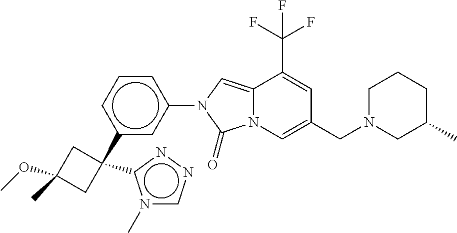 |
| 440 | 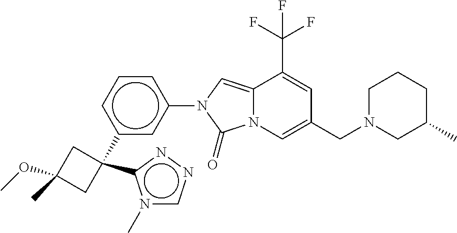 |
| 441 | 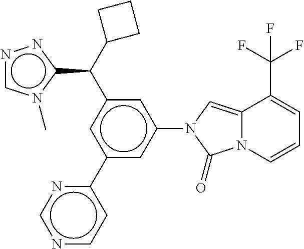 |
| 442 | 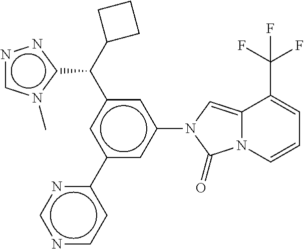 |
| 443 | 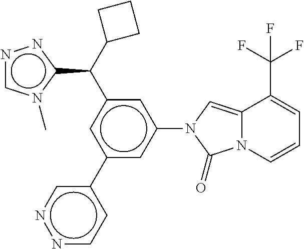 |
| 444 | 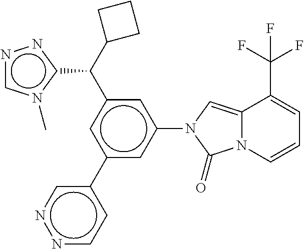 |
| 445 | 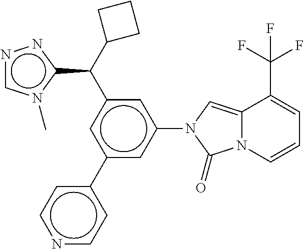 |
| 446 | 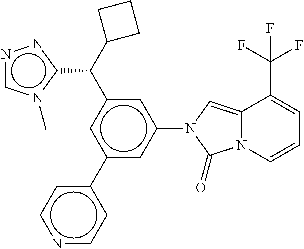 |
| 447 | 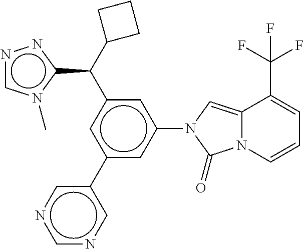 |
| 448 | 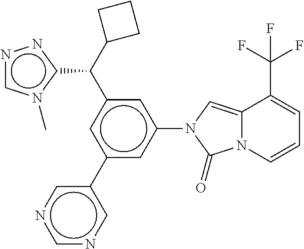 |
| 449 | 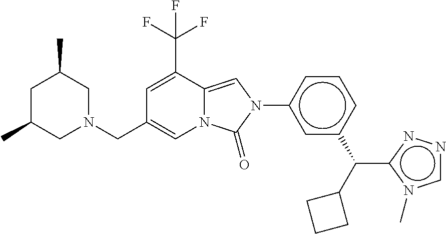 |
| 450 | 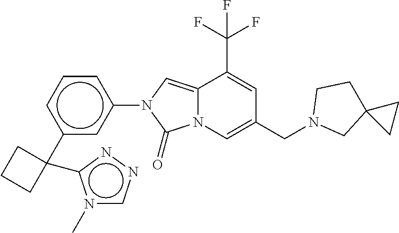 |
| 451 | 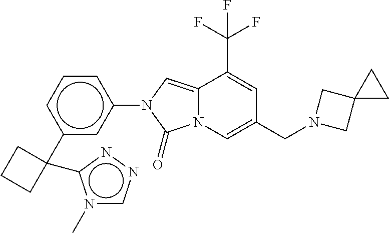 |
| 452 | 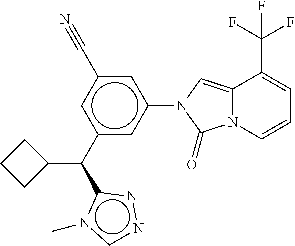 |
| 453 | 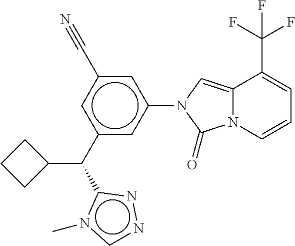 |
| 454 | 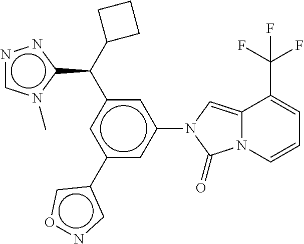 |
| 455 | 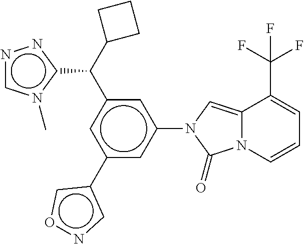 |
| 456 | 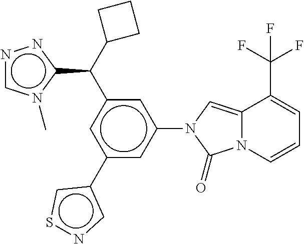 |
| 457 | 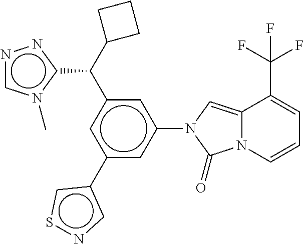 |
| 458 | 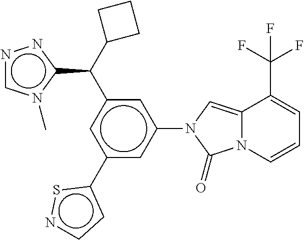 |
| 459 | 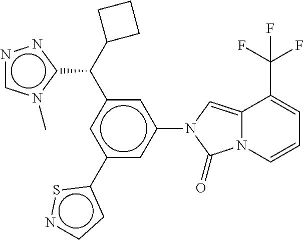 |
| 460 | 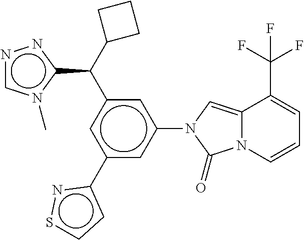 |
| 461 | 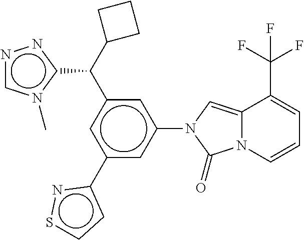 |
| 462 | 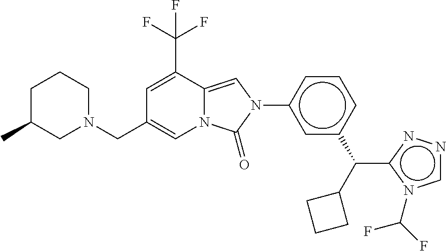 |
| 463 | 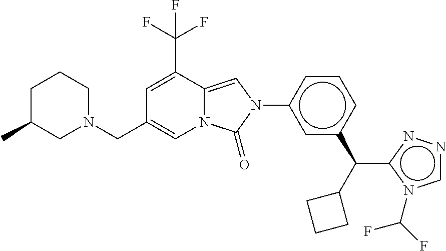 |
| 464 | 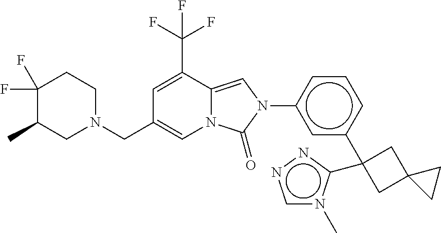 |
| 465 | 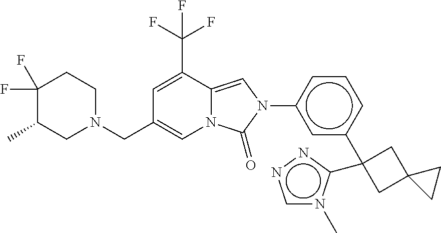 |
| 466 | 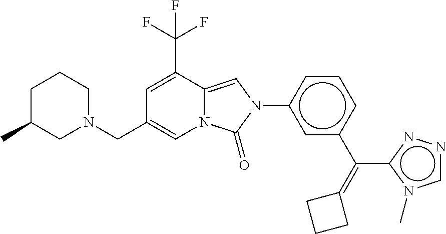 |
| 467 | 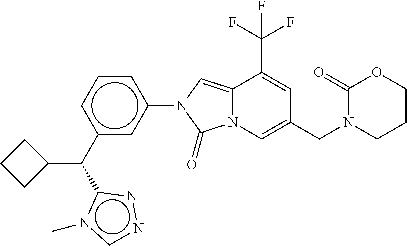 |
| 468 | 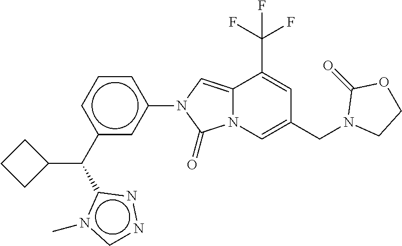 |
| 469 | 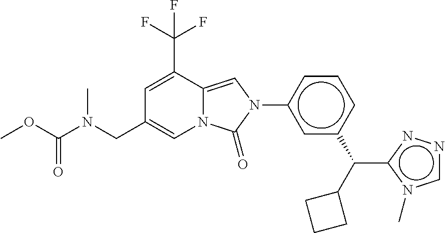 |
| 470_P1 | 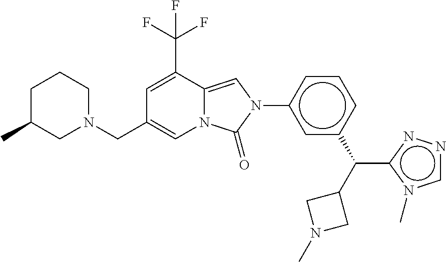 |
| 470_P2 | 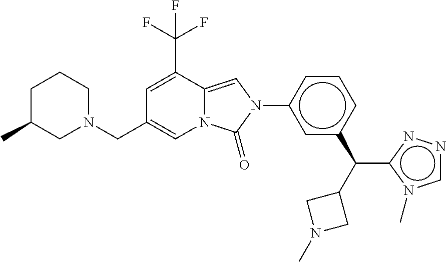 |
| 471 | 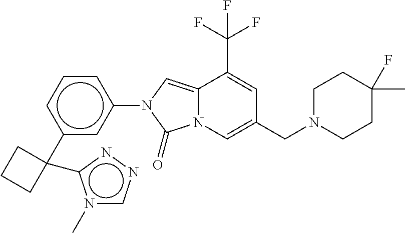 |
| 472_P1 | 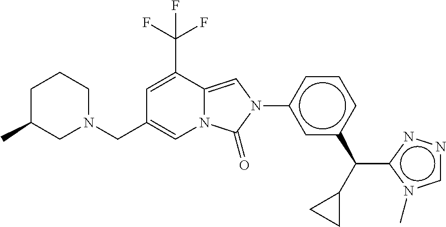 |
| 472_P2 | 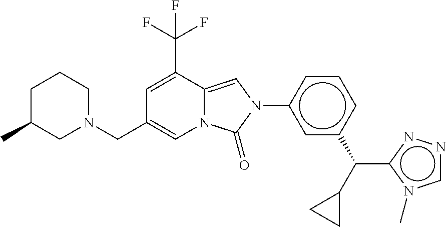 |
| 473 | 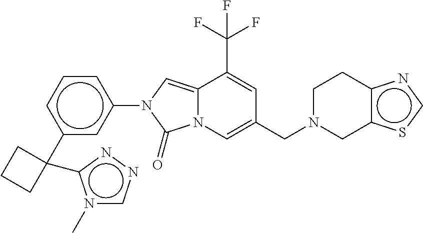 |
| 474 | 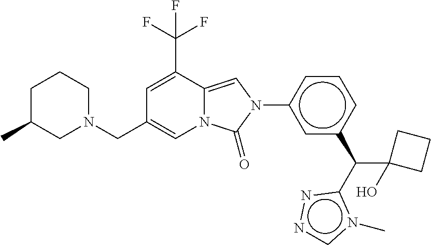 |
| 475 | 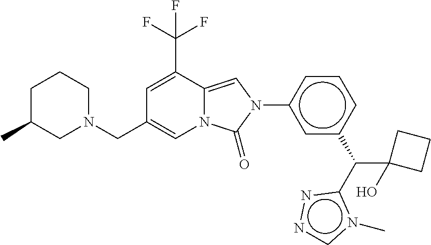 |
| 476 | 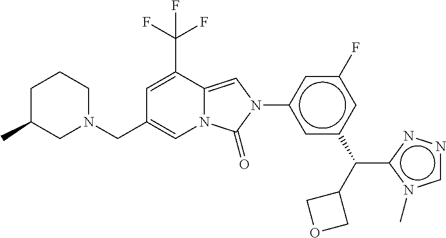 |
| 477 | 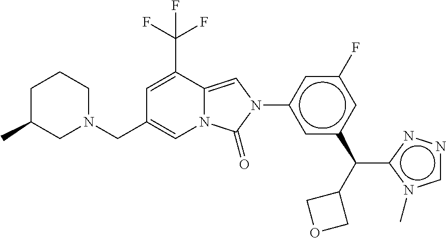 |
| 478 | 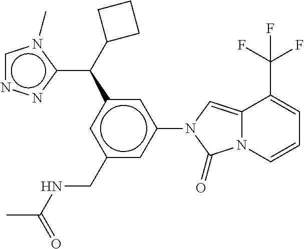 |
| 479 | 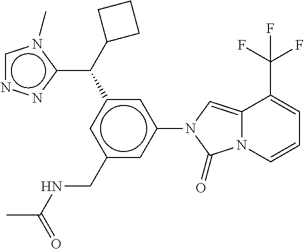 |
| 480 | 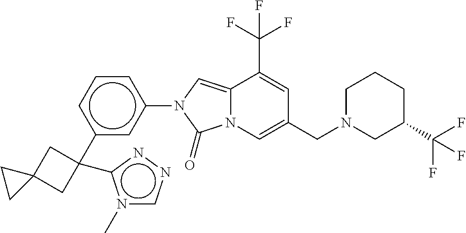 |
| 481 | 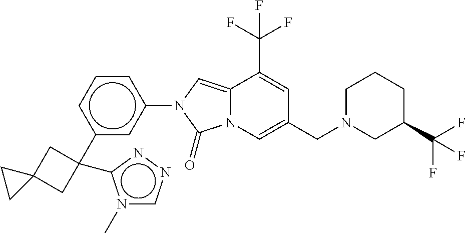 |
| 482 | 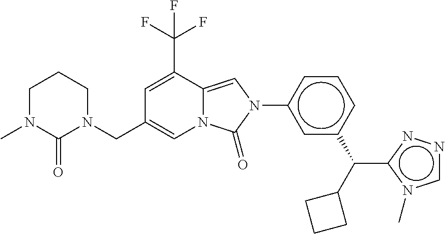 |
| 483 | 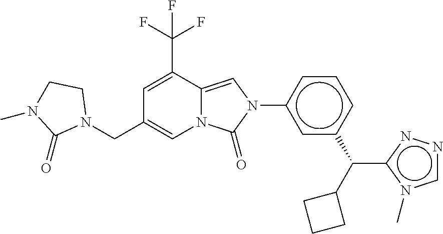 |
| 484 | 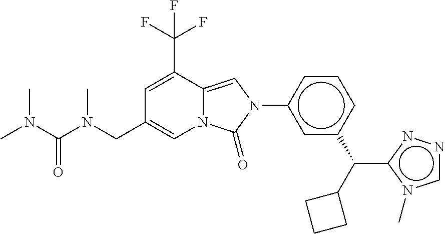 |
| 485 | 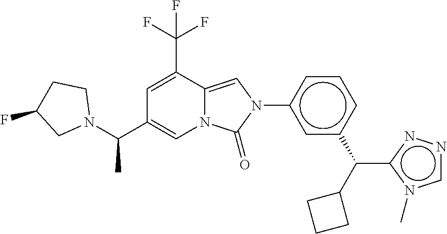 |
| 486 | 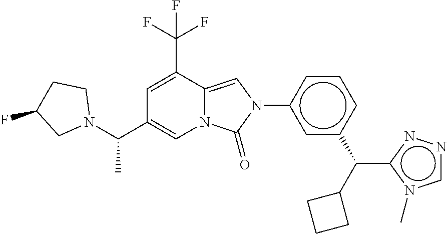 |
| 487 | 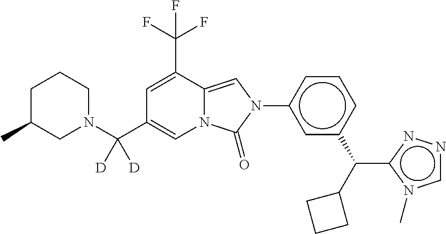 |
| 488 | 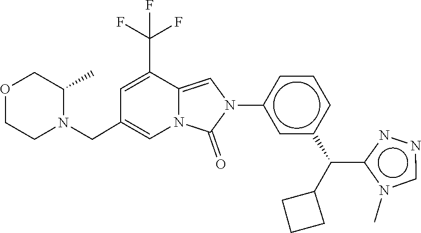 |
| 489 | 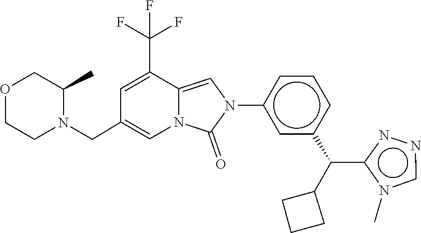 |
| 490 | 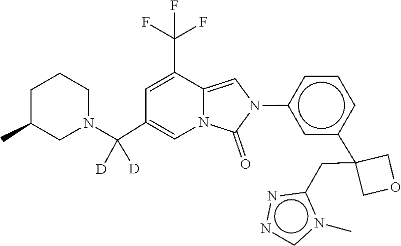 |
| 491 | 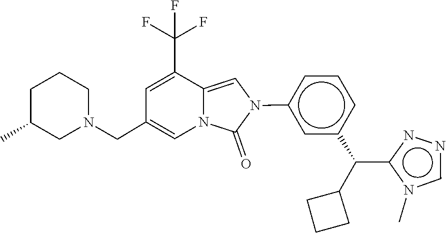 |
| 492 | 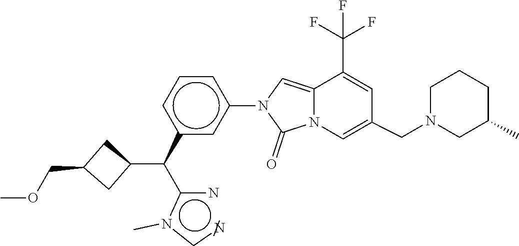 |
| 493 | 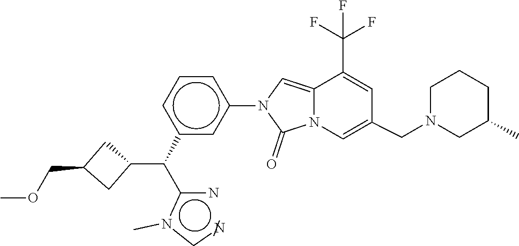 |
| 494 | 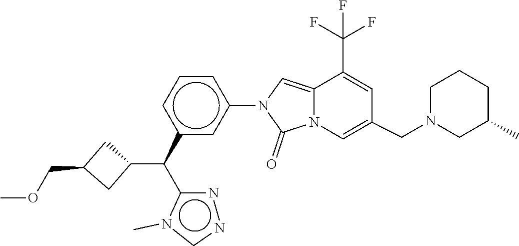 |
| 495 | 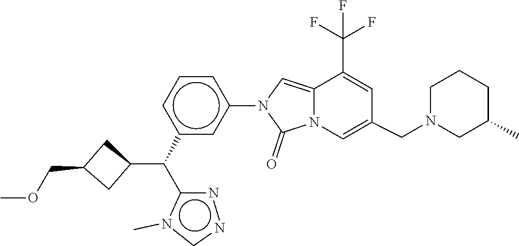 |
| 496 | 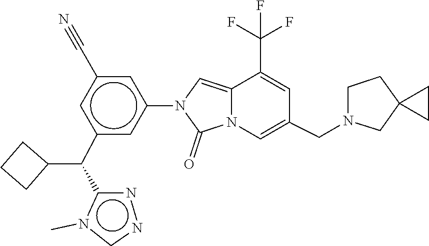 |
| 497 | 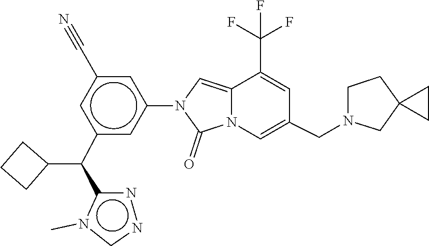 |
| 498 | 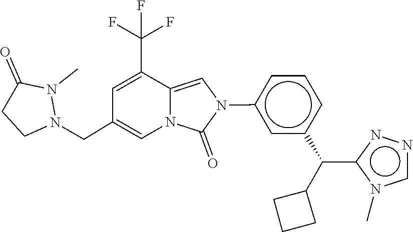 |
| 499 | 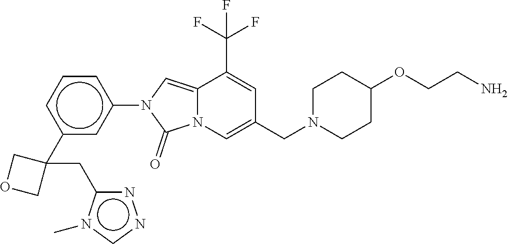 |
| 500 | 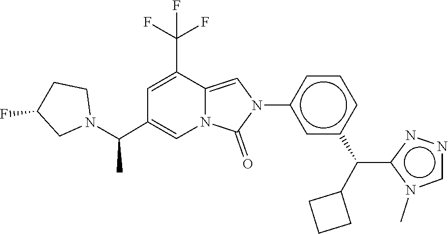 |
| 501 | 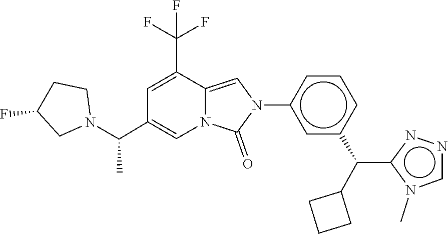 |
| 502 | 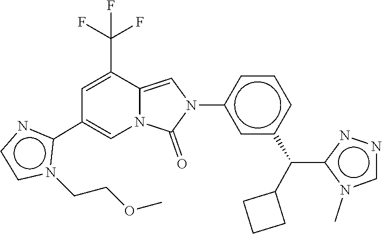 |
| 503 | 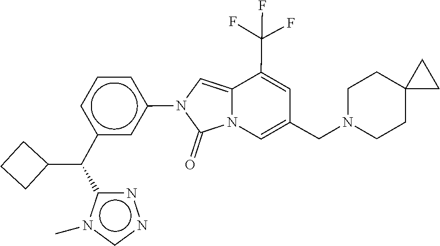 |
| 504 | 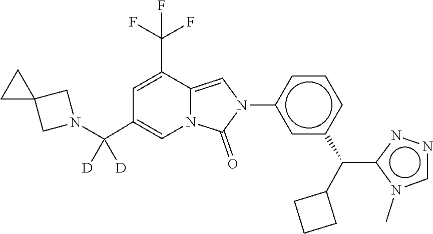 |
| 505 | 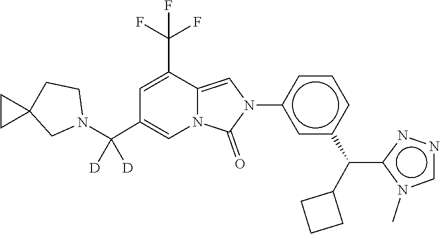 |
| 506_P1 | 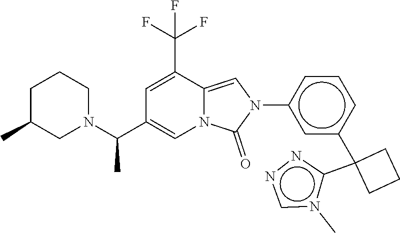 |
| 506_P2 | 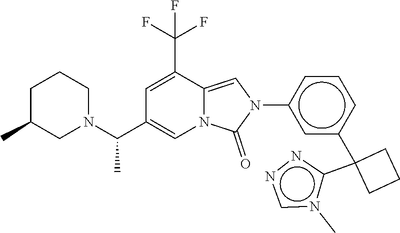 |
| 507 | 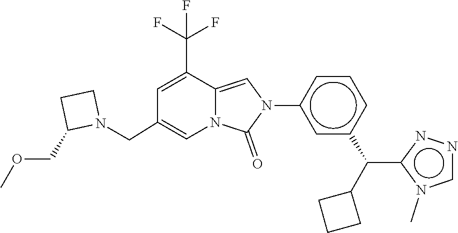 |
| 508 | 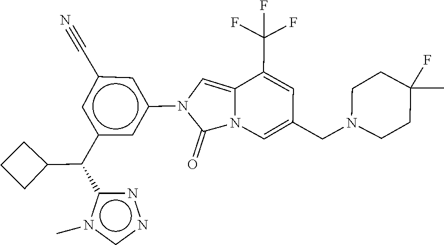 |
| 509 | 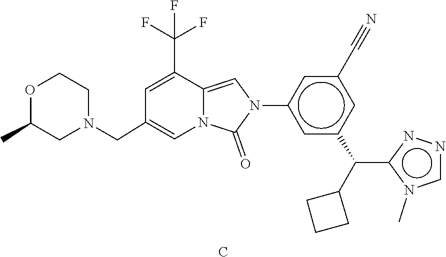 |
| 510 | 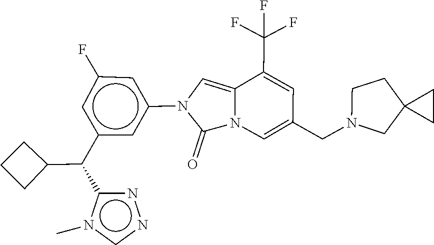 |
| 511 | 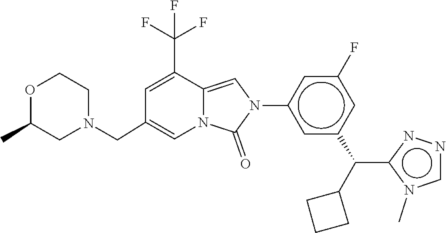 |
| 512 | 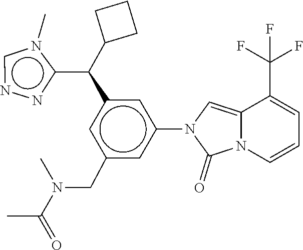 |
| 513 | 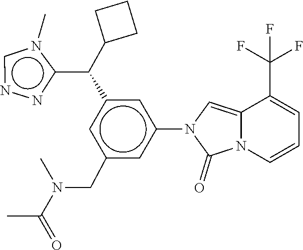 |
| 514 | 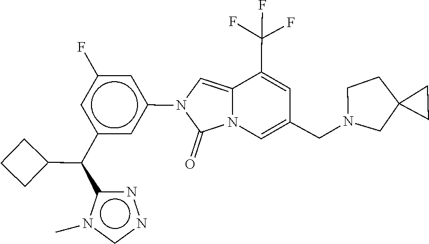 |
| 515 | 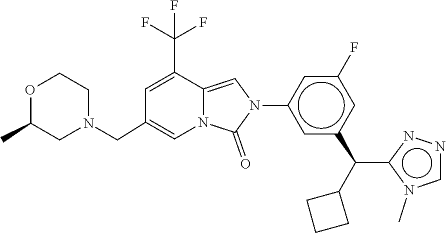 |
| 516 | 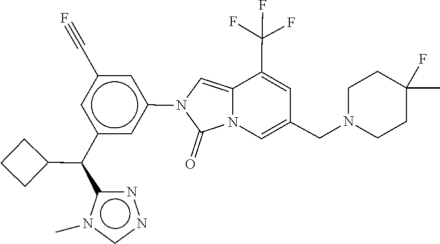 |
| 517 | 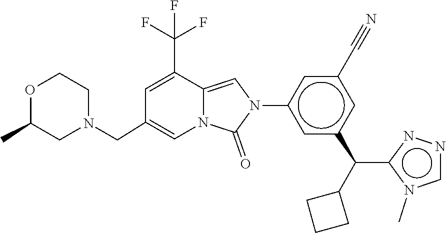 |
| 518 | 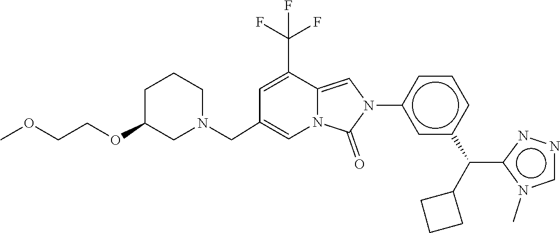 |
| 519 | 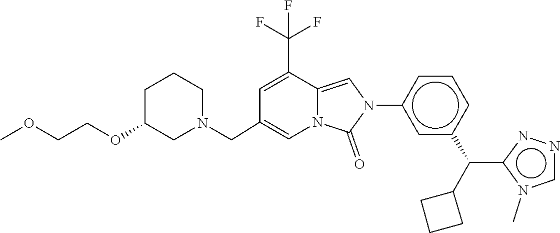 |
| 520 | 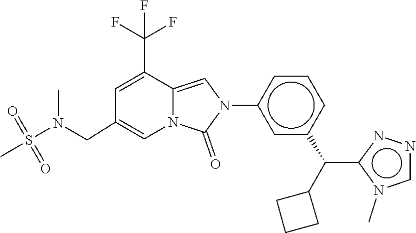 |
| 521 | 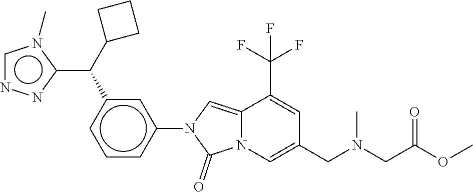 |
| 522 | 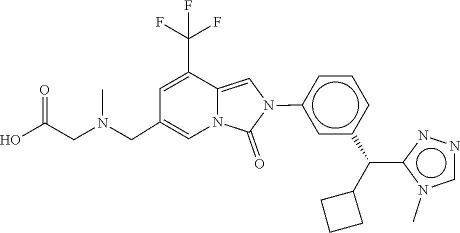 |
| 523 | 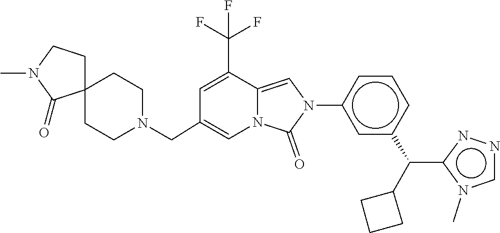 |
| 524 | 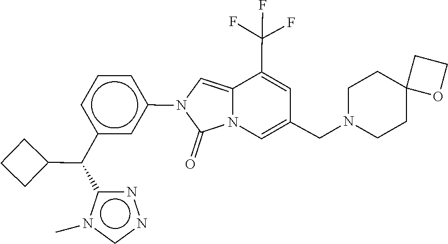 |
| 525 | 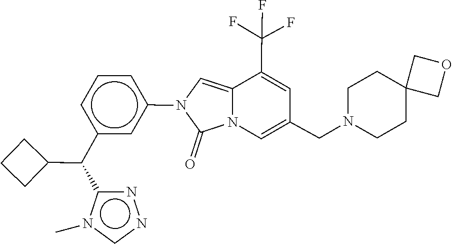 |
| 526 | 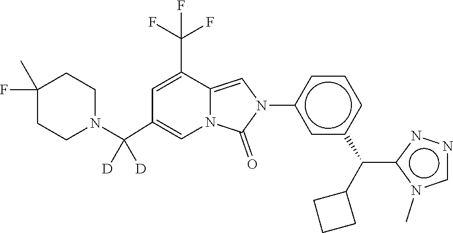 |
| 527 | 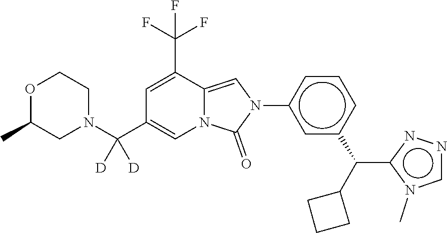 |
| 528 | 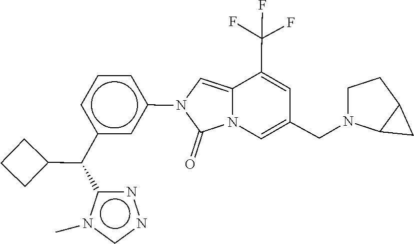 |
| 529 | 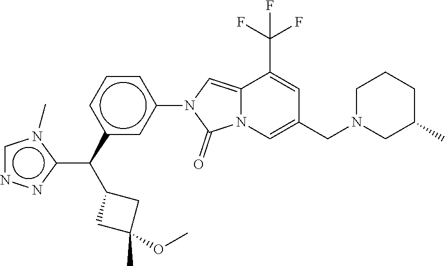 |
| 530 | 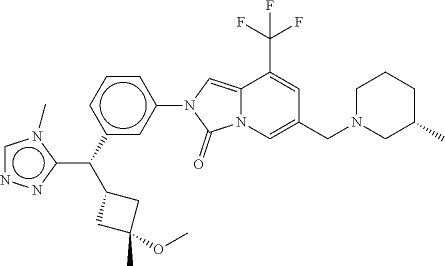 |
| 531 | 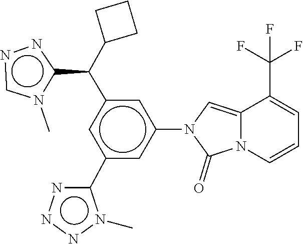 |
| 532 | 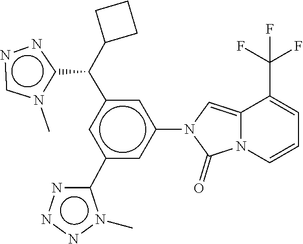 |
| 533 | 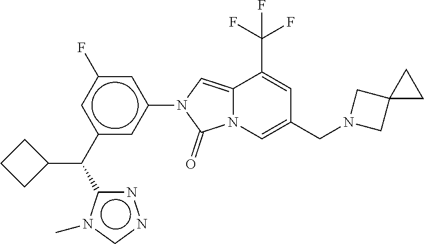 |
| 534 | 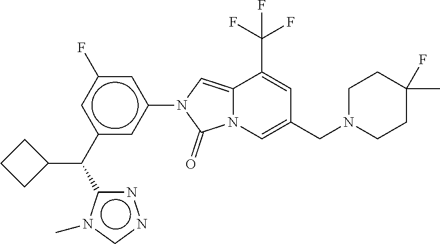 |
| 535 | 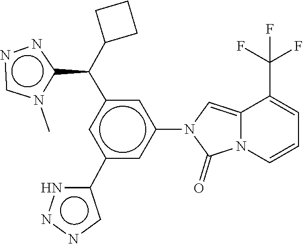 |
| 536 | 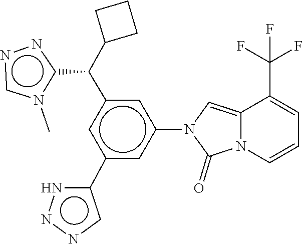 |
| 537 | 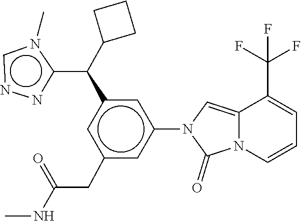 |
| 538 | 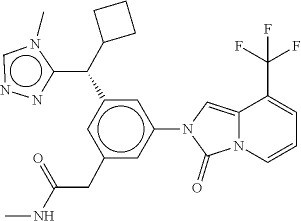 |
| 539 | 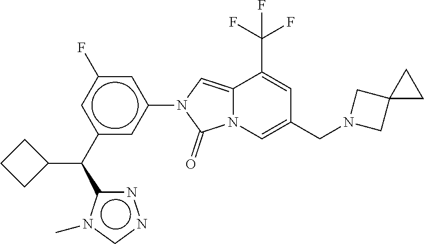 |
| 540 | 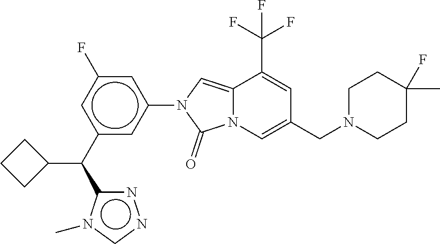 |
| 541 | 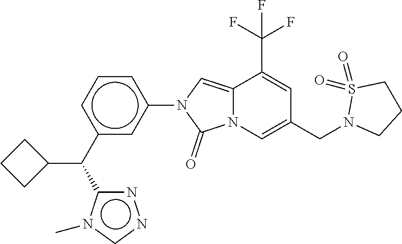 |
| 542 | 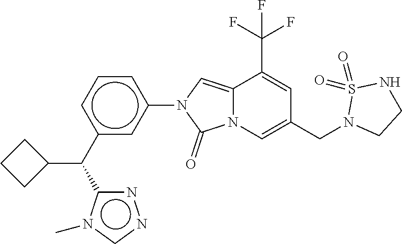 |
| 543 | 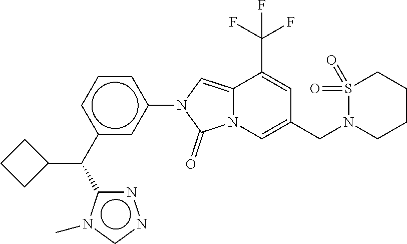 |
| 544 | 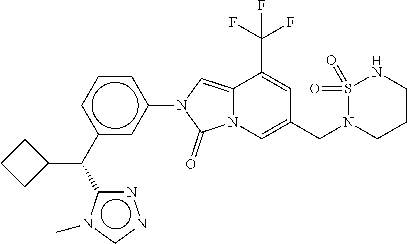 |
| 545 | 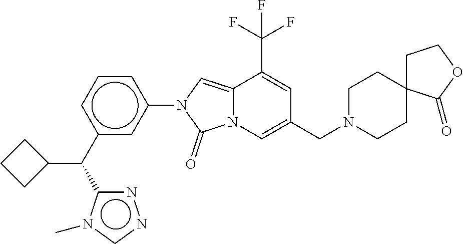 |
| 546 | 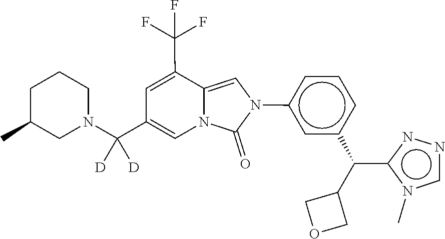 |
| 547 | 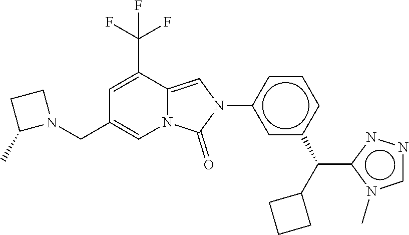 |
| 548 | 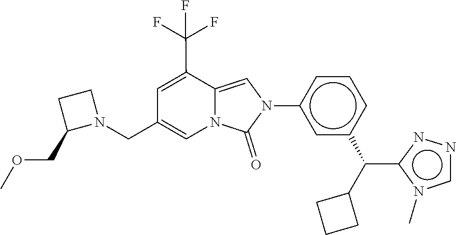 |
| 549 | 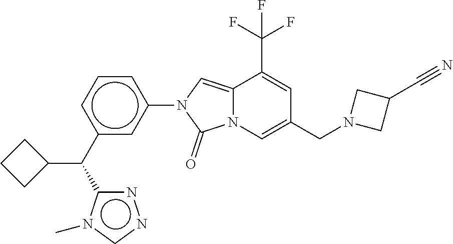 |
| 550_P1 | 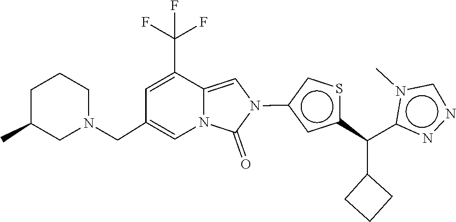 |
| 550_P2 | 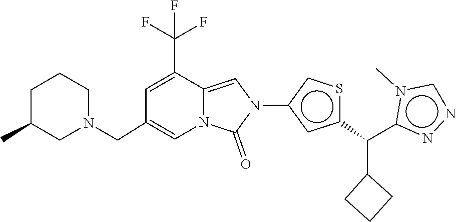 |
| 551 | 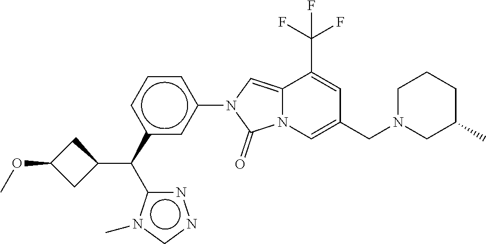 |
| 552 | 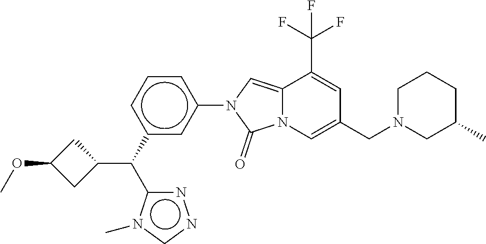 |
| 553 | 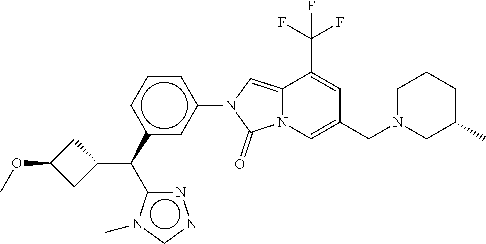 |
| 554 | 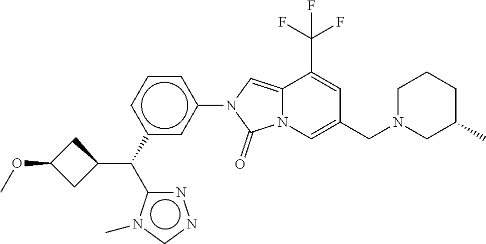 |
| 555 | 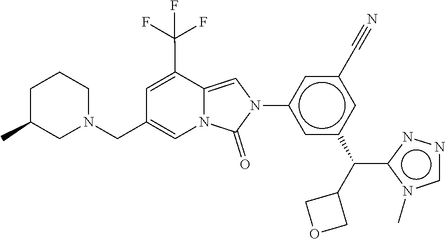 |
| 556 | 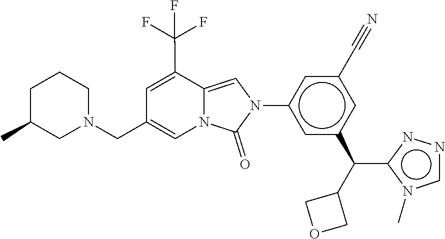 |
| 557 | 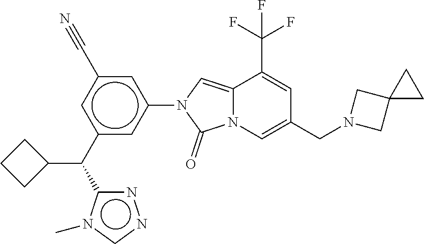 |
| 558 | 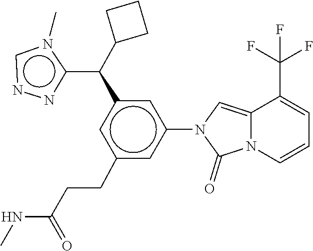 |
| 559 | 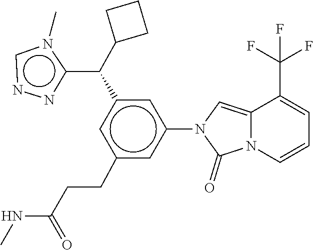 |
| 560 | 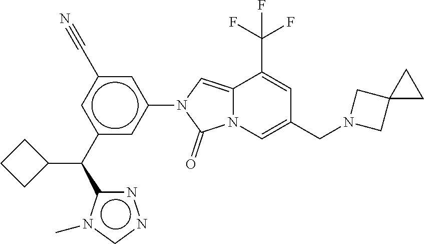 |
| 561 | 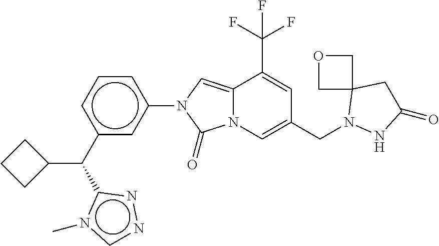 |
| 562 | 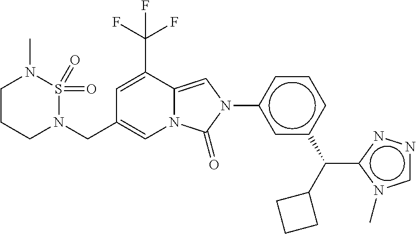 |
| 563 | 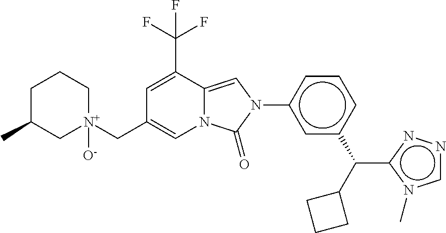 |
| 564 | 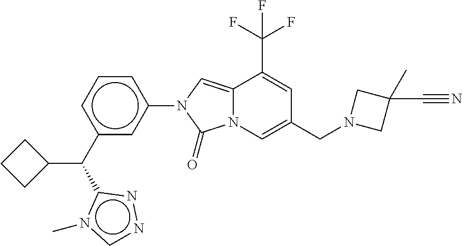 |
| 565_P1 | 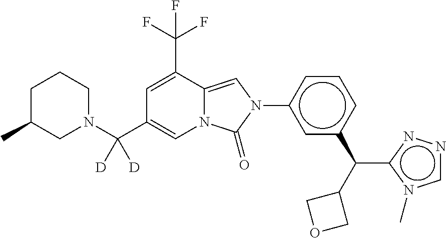 |
| 565_P2 | 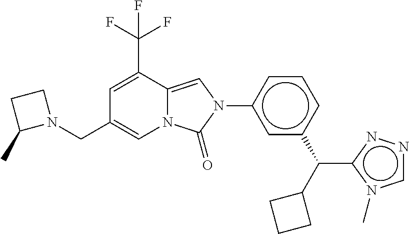 |
| 566 | 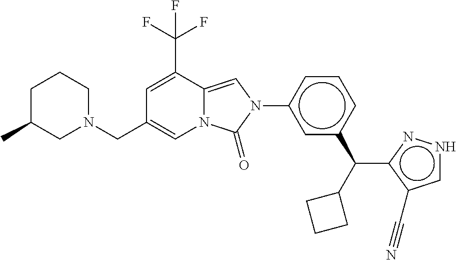 |
| 567 | 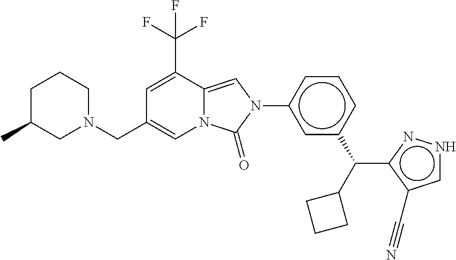 |
| 568 | 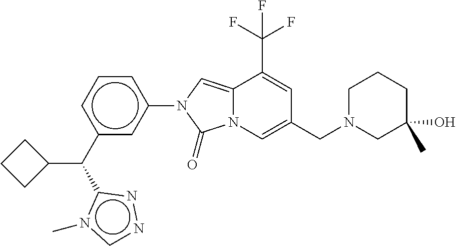 |
| 569 | 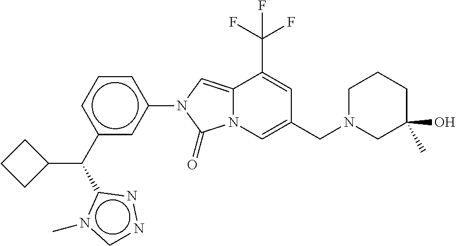 |
| 570 | 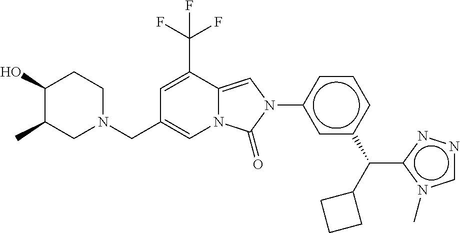 |
| 571 | 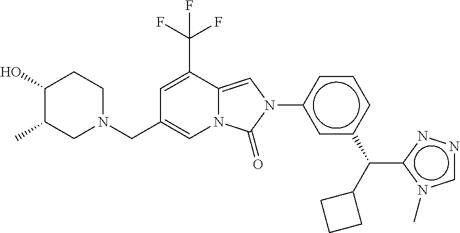 |
| 572 | 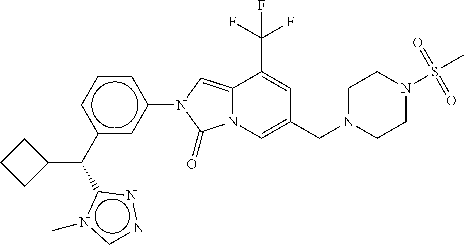 |
| 573_P1 | 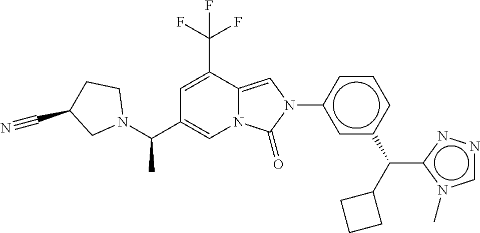 |
| 573_P2 | 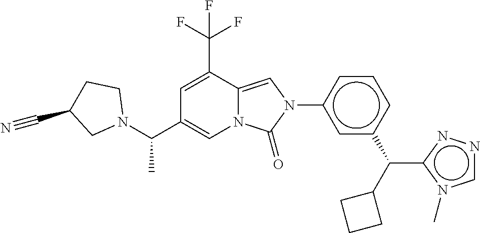 |
| 574 | 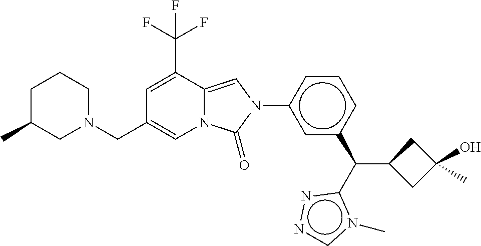 |
| 575 | 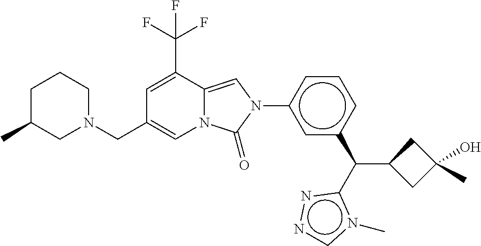 |
| 576 | 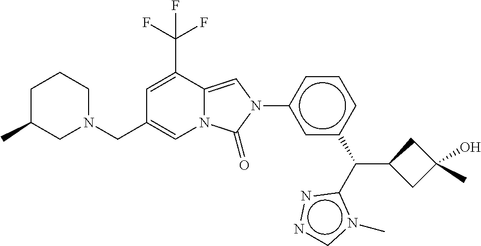 |
| 577 | 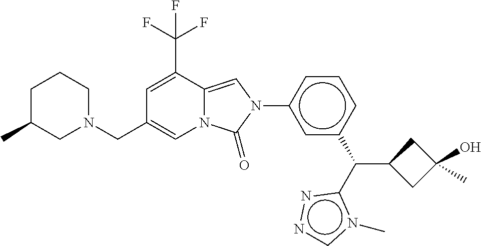 |
| 578 | 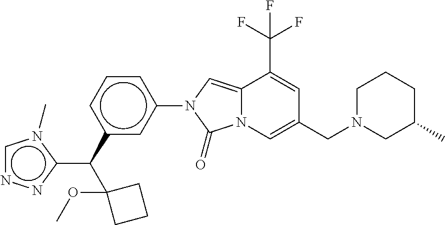 |
| 579 | 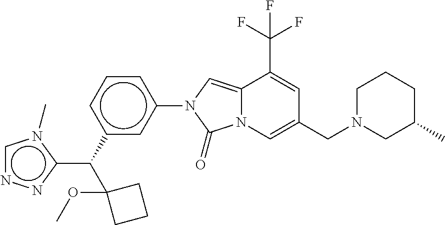 |
| 580 | 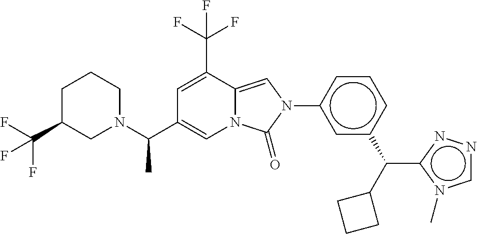 |
| 581 | 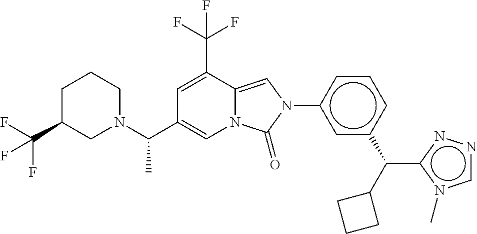 |
| 582 | 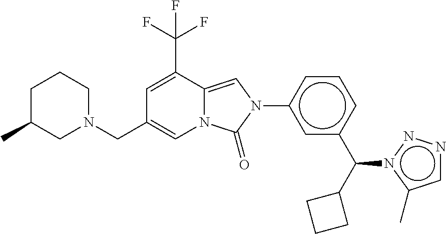 |
| 583 | 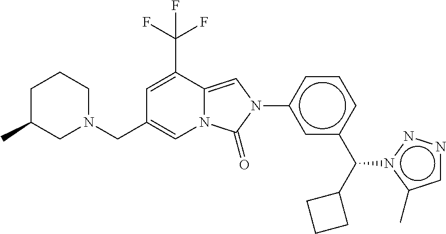 |
| 584 | 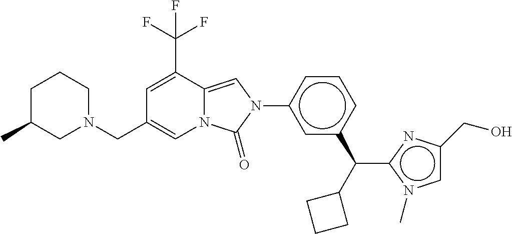 |
| 585 | 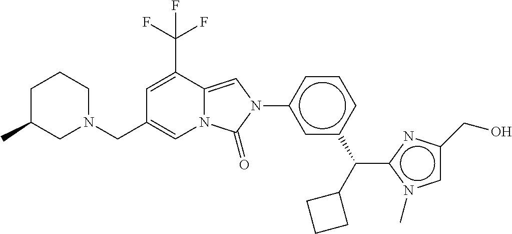 |
| 586 | 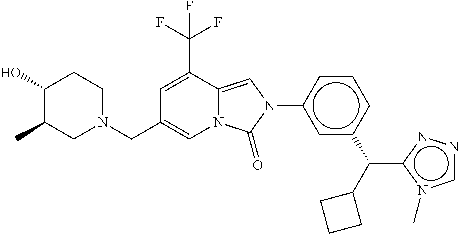 |
| 587 | 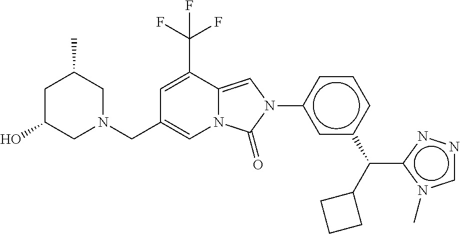 |
| 588 | 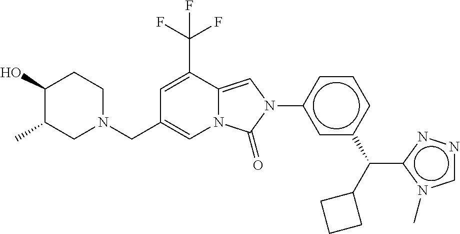 |
| 589 | 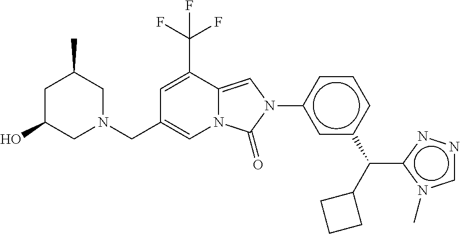 |
| 590 | 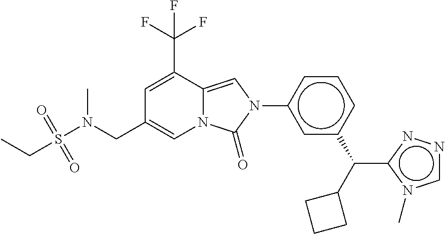 |
| 591 | 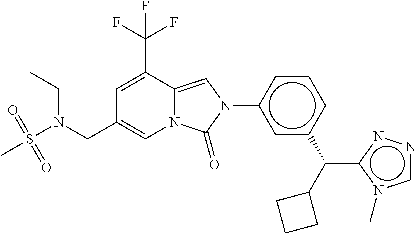 |
| 592 | 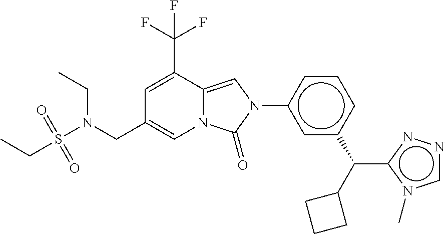 |
| 593 | 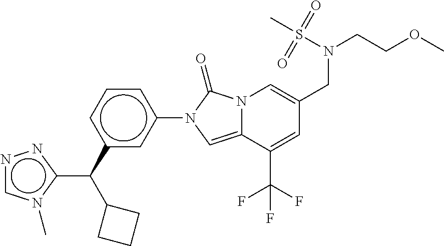 |
| 594 | 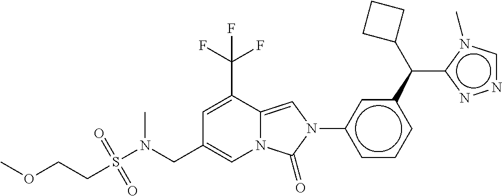 |
| 595 | 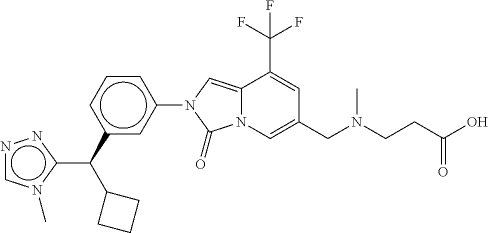 |
| 596 | 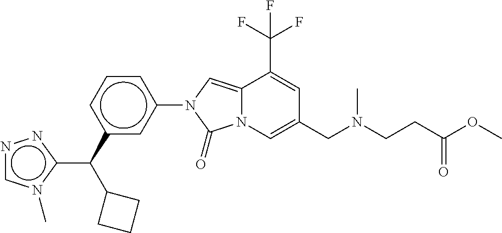 |
| 597_P1 | 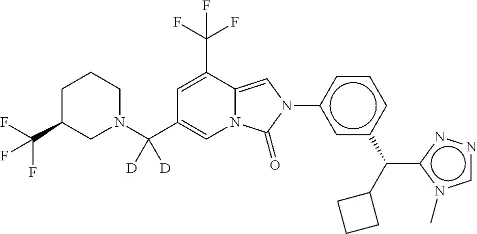 |
| 597_P2 | 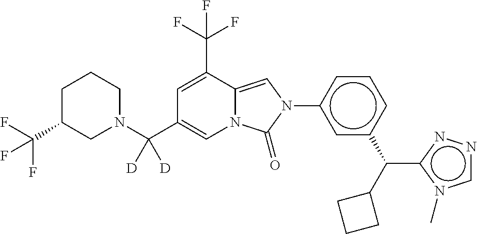 |
| 598 | 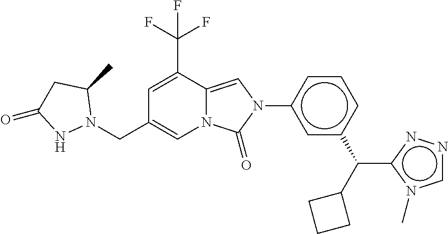 |
| 599 | 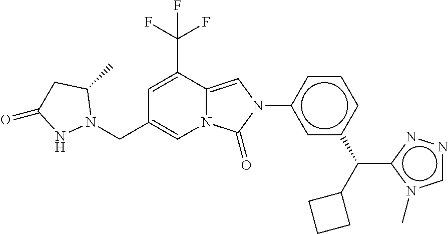 |
| 600 | 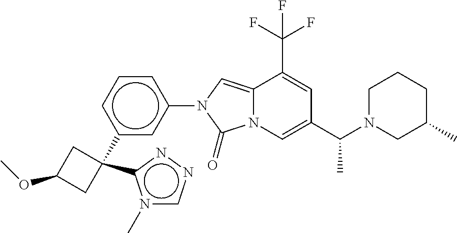 |
| 601 | 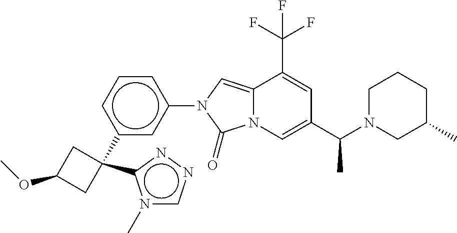 |
| 602 | 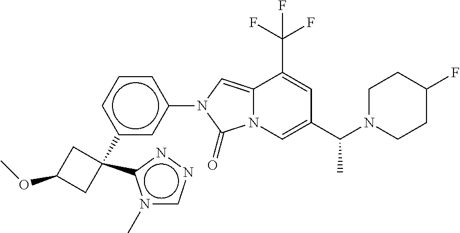 |
| 603 | 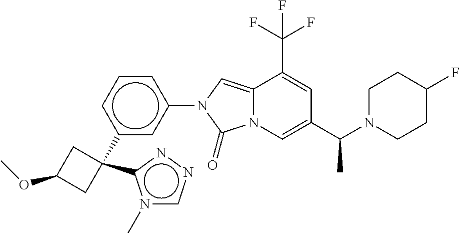 |
| 604 | 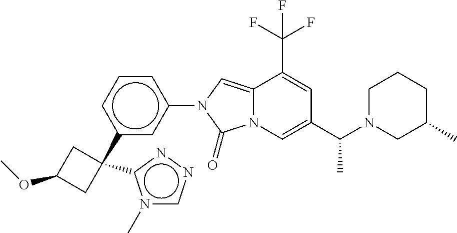 |
| 605 | 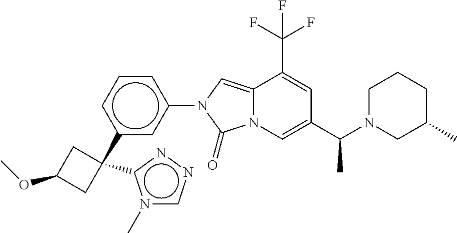 |
| 606 | 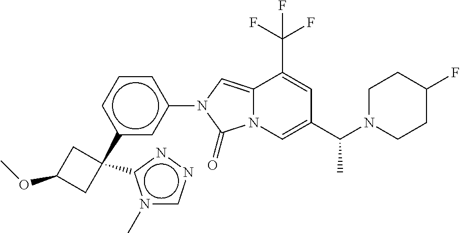 |
| 607 | 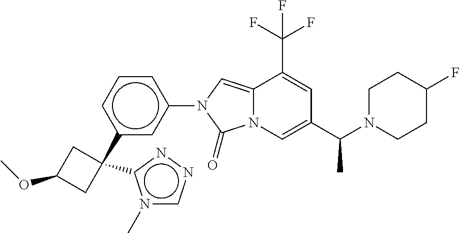 |
| 608 | 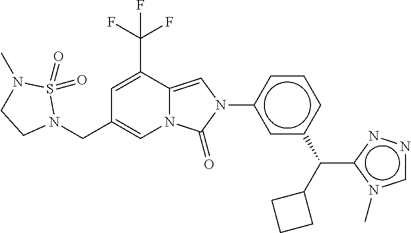 |
| 609 | 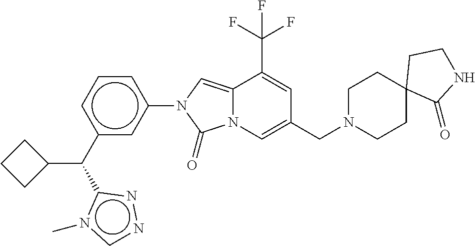 |
| 610_P1 | 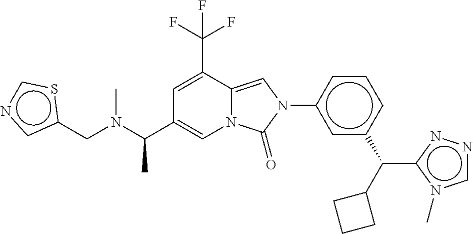 |
| 610_P2 | 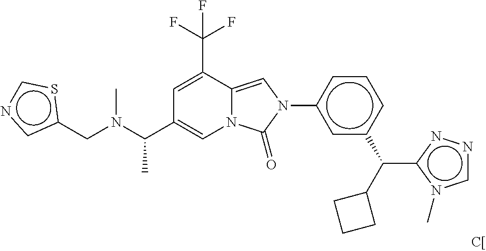 |
| 611 | 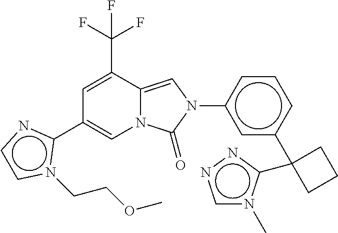 |
| 612_P1 | 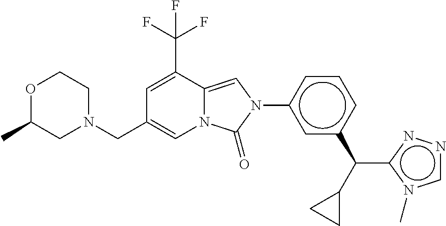 |
| 612_P2 | 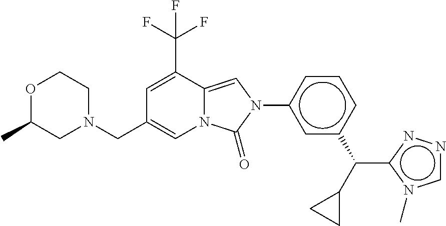 |
| 613 | 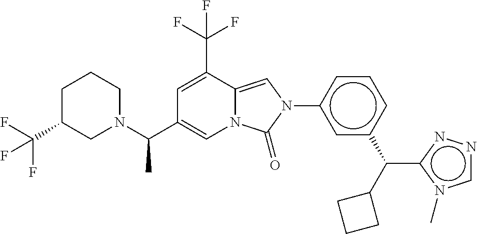 |
| 614 | 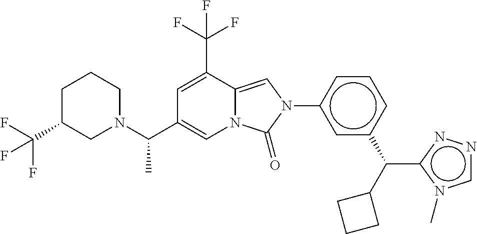 |
| 615 | 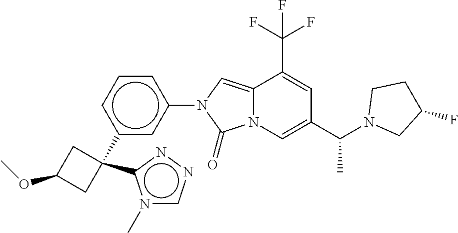 |
| 616 | 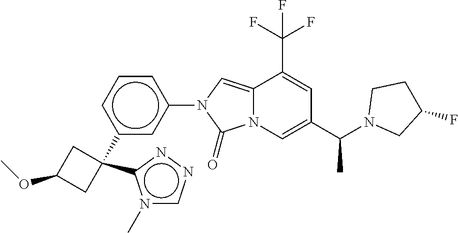 |
| 617 | 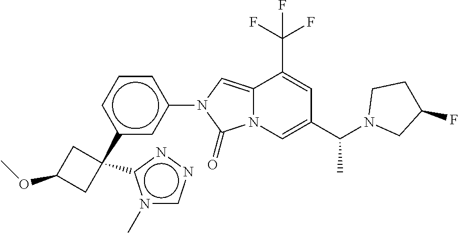 |
| 618 | 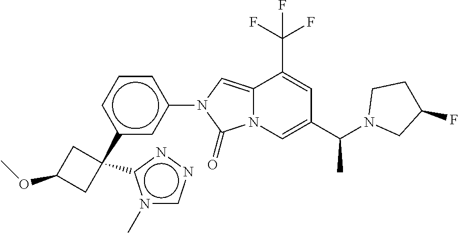 |
| 619 | 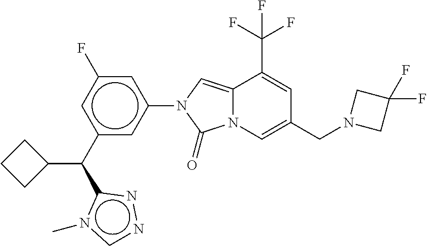 |
| 620 | 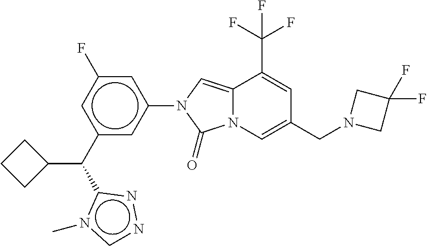 |
| 621 | 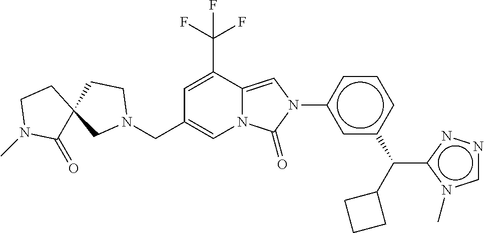 |
| 622 | 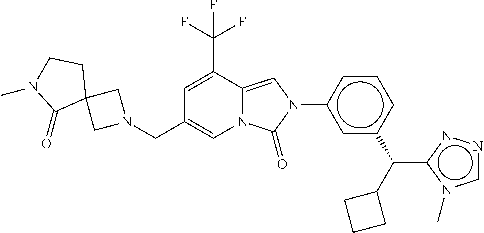 |
| 623 | 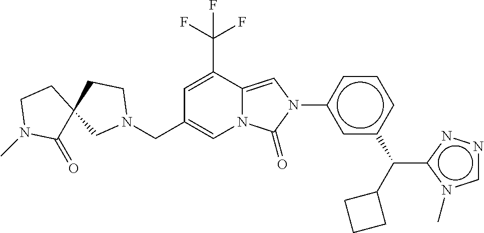 |
| 624 | 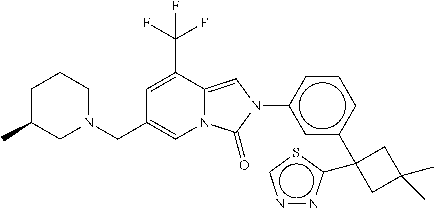 |
| 625 | 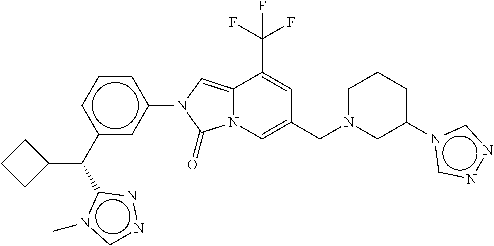 |
| 626 | 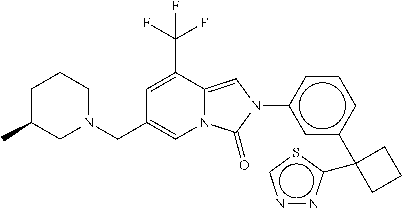 |
| 627_P1 | 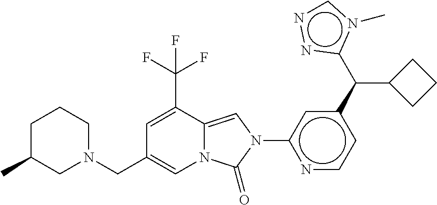 |
| 627_P2 | 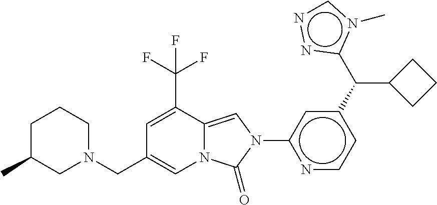 |
| 628_P1 | 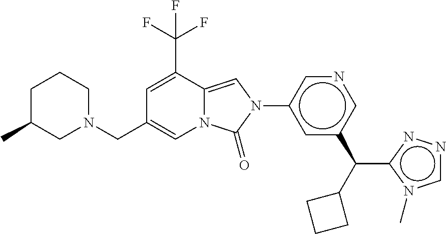 |
| 628_P2 | 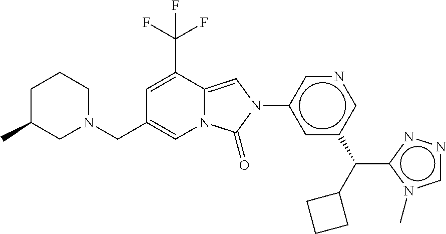 |

or a pharmaceutically acceptable salt thereof.

22. A pharmaceutical composition comprising a compound of any of claims 1-21 and a pharmaceutically acceptable adjuvant or carrier.

23. A method of treating a disease or condition associated with cell proliferation comprising administering a therapeutically effective amount of a compound of any of claims 1-21 or a pharmaceutical composition of claim 22 to a subject in need thereof.

24. The method of claim 23, wherein the disease or condition associated with cell proliferation is hyperplasia or cancer.

25. The method of claim 24, wherein cancer is a hematologic cancer.

26. The method of claim 25, wherein the hematologic cancer is selected from a group consisting of lymphoma, leukemia, and myeloma.

27. The method of claim 26, wherein cancer is a non-hematologic cancer.

28. The method of claim 27, wherein the non-hematologic cancer is a sarcoma or a carcinoma.

29. The method of any one of claims 23-28, wherein the subject has one or more of increased T-cell activation, increased T-cell proliferation, decreased T-cell exhaustion, decreased T-cell anergy and decreased T-cell tolerance after administration of compound of any of claims 1-15 or a pharmaceutical composition of claim 16.

30. The method of claim 29, wherein increased T-cell activation comprises increased production of a cytokines.

31. The method of claims 23-28, wherein the subject has increased NK-cell activation.

32. The method of 31, the increased NK-cell activation comprises increased production of cytokines.
